(19) United States
(12) Patent Application Publication
(10) Pub. No.: US 2025/0388565 A1
(43) Pub. Date: Dec. 25, 2025

(54) ESTROGEN RECEPTOR ALPHA DEGRADERS AND METHODS OF USE THEREOF

(71) Applicant: Relay Therapeutics, Inc., Cambridge, MA (US)

(72) Inventors: Alexander M. Taylor, Cambridge, MA (US); Kurt Armbrust, Cambridge, MA (US); Christopher A. Lepre, Cambridge, MA (US); Thomas H. McLean, Cambridge, MA (US); Paul R. Fleming, Cambridge, MA (US); Rulin MA, Cambridge, MA (US); Erich W. Baum, Cambridge, MA (US); Cary Griffin Fridrich, Cambridge, MA (US); Jing He, Cambridge, MA (US); Charles R. Heap, Cambridge, MA (US); Jay F. Larrow, Cambridge, MA (US); Rukundo Ntaganda, Cambridge, MA (US)

(21) Appl. No.: 18/879,705

(22) PCT Filed: Jun. 27, 2023
(86) PCT No.: PCT/US2023/069200

§ 371 Date: Dec. 27, 2024

Related U.S. Application Data
(60) Provisional application No. 63/355,861, filed on Jun. 27, 2022.
(60) Provisional application No. 63/398,067, filed on Aug. 15, 2022.
(60) Provisional application No. 63/405,388, filed on Sep. 9, 2022.
(60) Provisional application No. 63/435,063, filed on Dec. 23, 2022.

(51) Int. Cl. C07D 401/14; C07D 405/12; C07D 405/14; C07D 471/10; C07D 487/04

(52) U.S. Cl. CPC C07D 401/14; C07D 405/12; C07D 405/14; C07D 471/10; C07D 487/04

(57) ABSTRACT

The present disclosure relates to novel compounds and pharmaceutical compositions thereof, and methods for degrading ERα with the compounds and compositions of the disclosure. The present disclosure further relates to, but is not limited to, methods for treating disorders associated with ERα-mediated transcription regulation with the compounds and compositions of the disclosure.

### CROSS-REFERENCE TO RELATED APPLICATIONS

This application claims the benefit of and priority to U.S. Provisional Application No. 63/355,861 filed Jun. 27, 2022; U.S. Provisional Application No. 63/398,067 filed Aug. 15, 2022; U.S. Provisional Application No. 63/405,388 filed Sep. 9, 2022; and U.S. Provisional Application No. 63/435,063 filed Dec. 23, 2022, the contents of each of which are incorporated herein by reference in their entirety

### TECHNICAL FIELD OF THE INVENTION

The present invention relates to compounds and methods useful for the modulation of estrogen receptor alpha (“ERα”) via ubiquitination and/or degradation by compounds according to the present invention. The invention also provides pharmaceutically acceptable compositions comprising compounds of the present invention and methods of using said compositions in the treatment of various disorders

### BACKGROUND OF THE INVENTION

The Estrogen Receptors (ER) are members of the nuclear hormone receptor superfamily. Two classes of ER exist: estrogen receptor alpha (ERα) and estrogen receptor beta (ERB), encoded by the ESR1 and the ESR2 genes respectively. ERα and ERβ are ligand-activated transcription regulators activated by the hormone estrogen (e.g. 17β-estradiol). The ligand of ER, estrogen, is synthesized by the enzyme aromatase.

In the absence of estrogen, ERs are largely inactive and located in the cytosol of the cell. Upon estrogen binding, ERs migrate to the nucleus, form dimers and bind to specific genomic sequences called Estrogen Response Elements (ERE). ERs further recruit co-regulators to form a multi-protein complex that regulates the transcription of multiple target genes involved in the cellular proliferation and differentiation in target tissues.

Under physiological conditions, ERα expression is mainly restricted to reproductive tissues such as uterus, ovary, breast as well as bone and white adipose tissue. ERα is also expressed in more than 70% of breast cancer and is a major contributor to the pathophysiology of this cancer. Tumors harboring high levels of ERα are classified as ER-positive breast cancer. The etiological role of estrogen and ERα in breast cancer is well established and modulation of the ERα signaling pathway through endocrine therapy is a cornerstone of ER+ breast cancer treatment.

Currently, several strategies for inhibiting the estrogen/ERα signaling pathway in breast cancer exist: 1-Aromatase Inhibitors (AI), that act upstream of the ER signaling pathway by blocking estrogen production through inhibition of the aromatase enzyme and decreasing the levels of circulating estrogen; 2-Selective Estrogen Receptor Modulators (SERM) bind directly to ERα and competitively inhibit estrogen binding and thus antagonizing ERα activity; 3-Selective Estrogen Receptor Downregulators or Degradors (SERD) that both antagonize and degrade ERα. This process is mediated by induced-conformational changes and ERα protein degradation through the proteasome pathway; 4-Proteolysis-Targeting Chimeras (PROTAC) are heterobifunctional molecules that selectively recruit an E3 ubiquitin ligase to the ERα protein through induced proximity and mediate ubiquitination and proteasomal degradation of ERα.

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Bemdsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein binding ligand and an E3 ubiquitin ligase ligand induce proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17 (6): 551-555; Schnnekloth JS Jr., Chembiochem, 2005, 6 (1): 40-46).

De-novo and acquired resistance to endocrine therapies can arise through distinct mechanisms such as ERα coregulators overexpression or post-translational modification of ERα and its coregulators upon activation of intercellular signaling pathways. All contribute to hypersensitivity of ERα to low circulating estrogen levels. Additionally genomic alterations such as point mutations in the ESR1 gene or chromosomal translocation can result in the ability to bind to DNA in the absence of ligand and confer hormone independence in ERα mutated cancer cells. Since most of the endocrine therapy resistance mechanisms identified rely on ERα-dependent mechanisms, strategies aimed at downregulating ERα (both wild-type and mutant) through targeted protein degradation may overcome resistance and provide better treatment options.

An ongoing need exists in the art for effective treatments for disease, especially hyperplasias and cancers, such as breast cancer. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins such as estrogen receptor alpha (“ERα”) hold promise as therapeutic agents. Accordingly, there remains a need to find bifunctional compounds that are ERα degraders useful as therapeutic agents.

### SUMMARY OF THE INVENTION

The present application relates to novel bifunctional compounds, which function to recruit ERα to E3 Ubiquitin Ligase for degradation, and methods of preparation and uses thereof. In particular, the present disclosure provides bifunctional compounds, which find utility as modulators of targeted ubiquitination of ERα, which is then degraded and/or otherwise inhibited by the bifunctional compounds as described herein. An advantage of the compounds provided herein is that a broad range of pharmacological activities is possible, consistent with the degradation/inhibition of ERα. In addition, the description provides methods of using an effective amount of the compounds as described herein for the treatment or amelioration of a disease condition, such as cancer, e.g., breast cancer.

The present application further relates to targeted degradation of ERα through the use of bifunctional molecules, including bifunctional molecules that link a cereblon-binding moiety to a ligand that binds ERα.

It has now been found that compounds of this invention, and pharmaceutically acceptable compositions thereof, are effective as degraders of ERα. Such compounds have the general formula I-1:

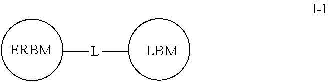

or a pharmaceutically acceptable salt thereof, wherein each variable is as defined and described herein.

In an embodiment, provided is a compound of formula I-3′:

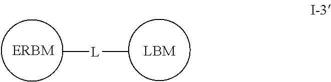

or a pharmaceutically acceptable salt thereof, wherein;
ERBM is selected from

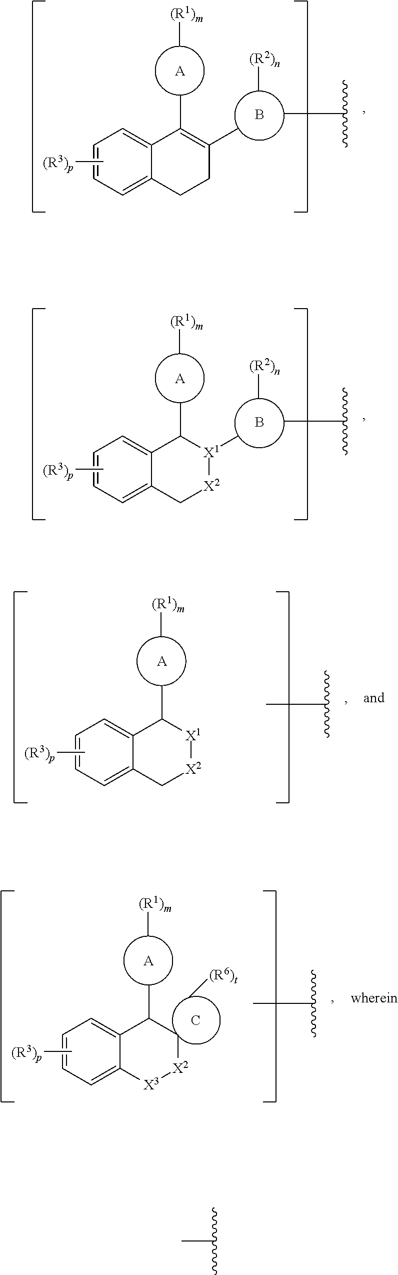

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A4} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl;
a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; 1,1′-biphenyl; naphthyl; tetrahydronaphthalenyl; dibydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

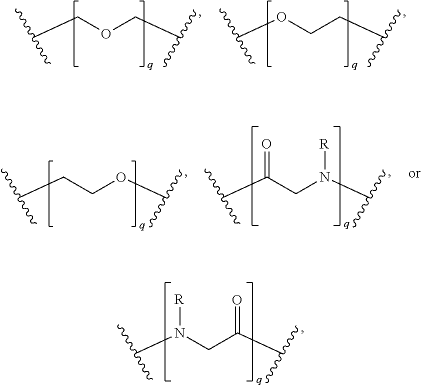

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

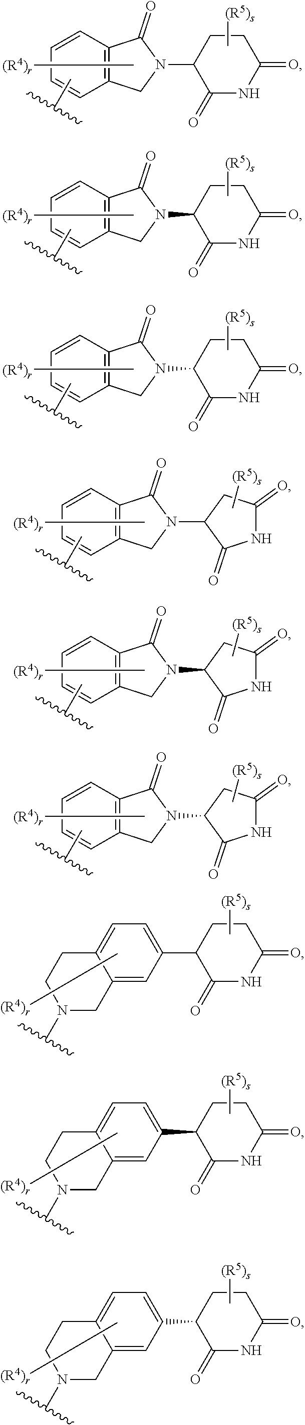

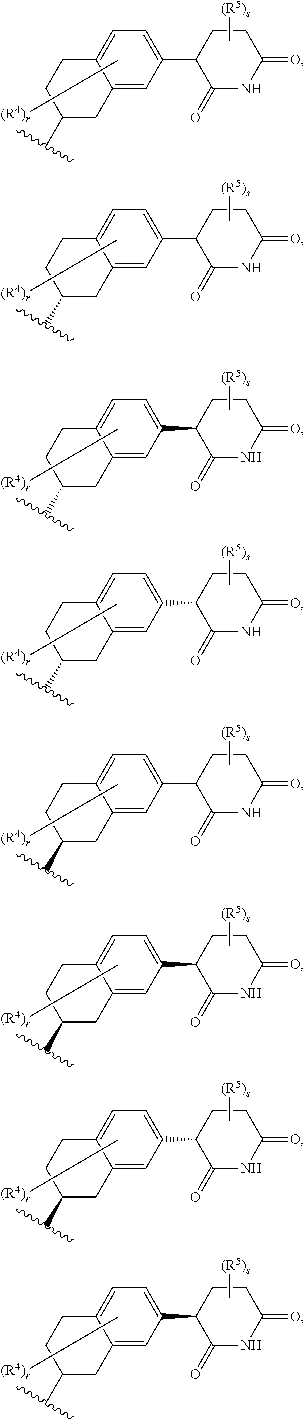

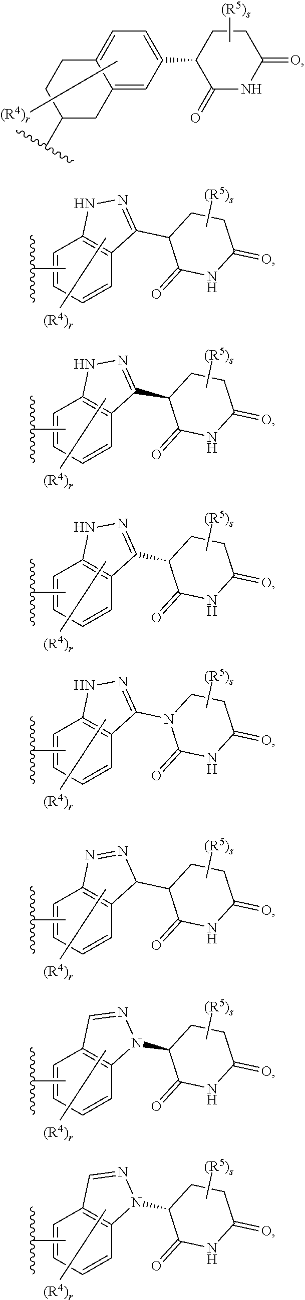

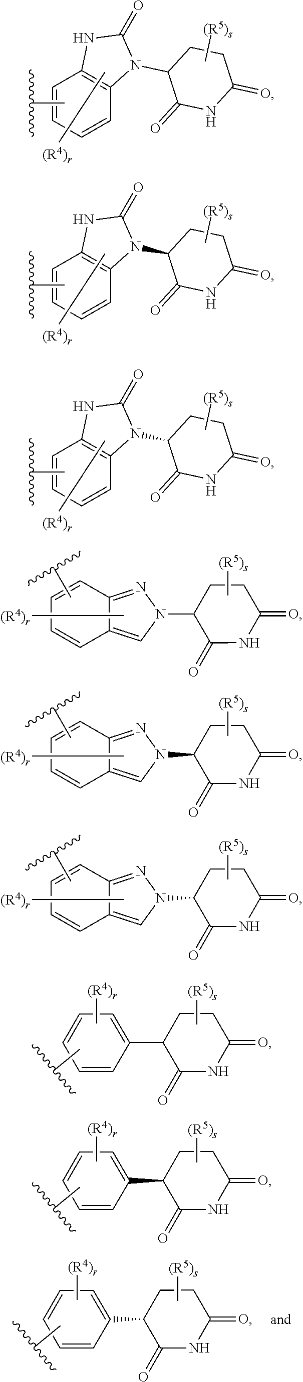

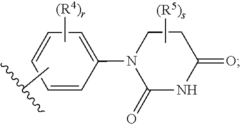

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

In an embodiment, provided is a compound of formula I-3′:

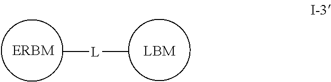

or a pharmaceutically acceptable salt thereof, wherein:
ERBM is selected from

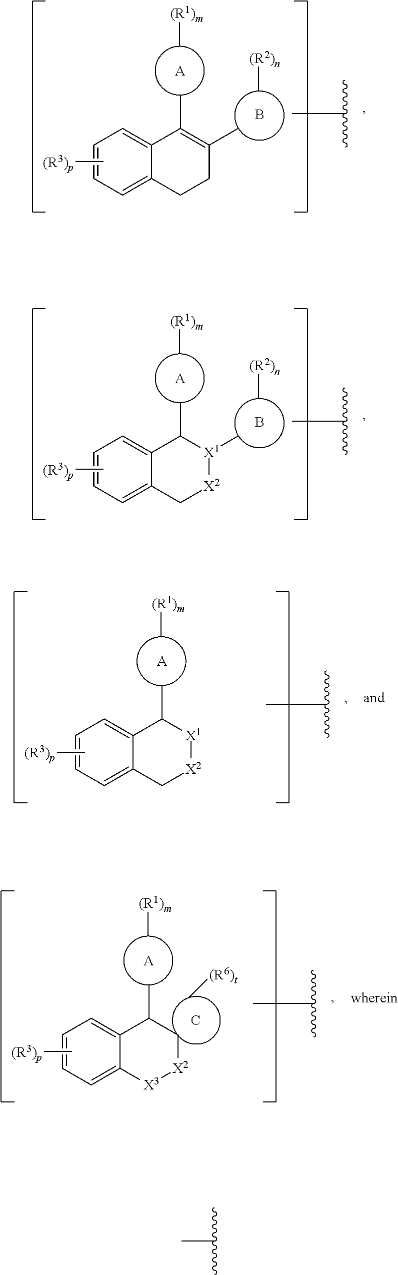

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents; X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A5} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;

L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

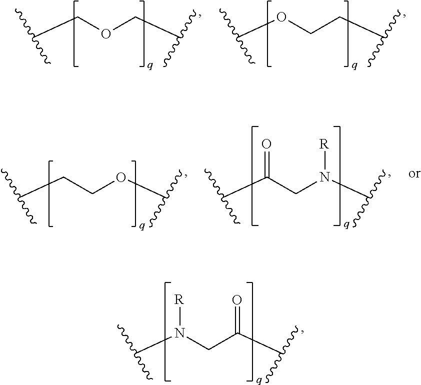

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

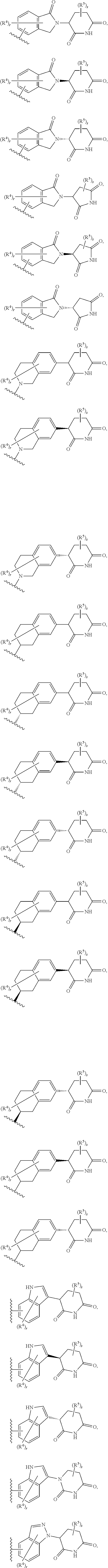

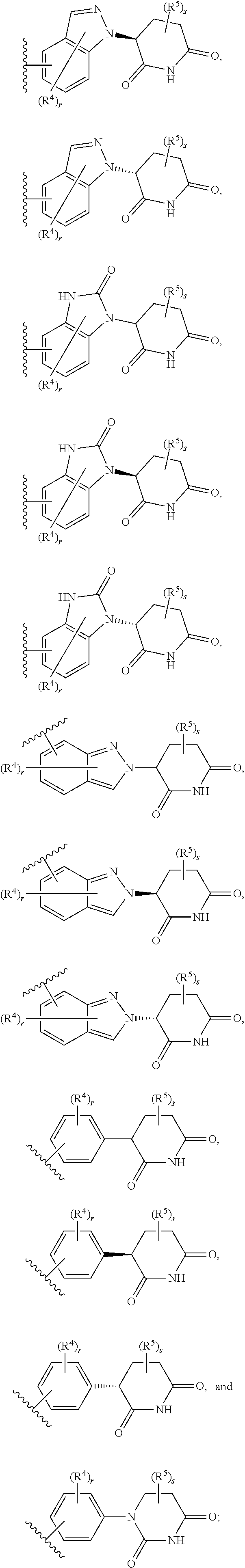

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4,
provided that when ERBM is

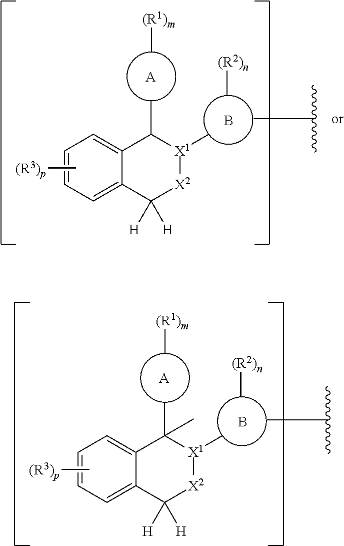

wherein X¹ is CH or N,
   X² is CH₂;
   Ring B is phenyl;
   Ring A is phenyl or a 6 membered monocyclic heteroaryl ring having 1-3 nitrogen heteroatoms;
   at least one R³ is —OH or —OMe;
   p is 1, 2, or 3;
   m is 0, 1 or 2;
   R¹ is selected from —F and —Cl;
   n is 0, 1 or 2; and
   R² is selected from —OH, -Me, —OMe, —F, —Br, —CF₃ and —ⁱPr;
then LBM is not

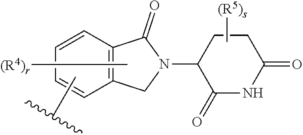

or a stereoisomer thereof.

Compounds of the present invention, and pharmaceutically acceptable compositions thereof, are useful for treating a variety of diseases, disorders, or conditions, associated with regulation of signaling pathways implicating ERα. Such diseases, disorders, or conditions include those described herein.

Compounds provided by this invention are also useful for the study of ERα enzymes in biological and pathological phenomena; the study of intracellular signal transduction pathways occurring in bodily tissues; and the comparative evaluation of new ERα inhibitors or ERα degraders or other regulators of ERα-mediated transcription in vitro or in vivo.

In some embodiments, the present disclosure provides a pharmaceutical composition comprising a compound of formula I-1, I-1′, I-2, I-2′, I-3 or I-3′ or a pharmaceutically acceptable salt thereof, and a pharmaceutically acceptable carrier, adjuvant, or diluent.

In some embodiments, the present disclosure provides a method of treating an ERα-mediated disorder comprising administering to a patient in need thereof a compound of formula I-1, I-1′, I-2, I-2′, I-3 or I-3′, or composition comprising said compound.

In some embodiments, the present disclosure provides a process for providing a compound of formula I-1, I-1′, I-2, I-2′, I-3 or I-3′, or synthetic intermediates thereof.

In some embodiments, the present disclosure provides a process for providing pharmaceutical compositions comprising compounds of formula I-1, I-1′, I-2, I-2′, I-3 or I-3′.

### DETAILED DESCRIPTION

### 1. General Description of Certain Embodiments of the Disclosure

Compounds of the present disclosure, and pharmaceutical compositions thereof, are useful as degraders of Erα.

In some embodiments, the present disclosure provides a compound of formula I-1:

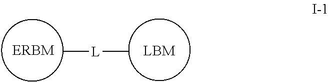

or a pharmaceutically acceptable salt thereof, wherein;
ERBM is selected from

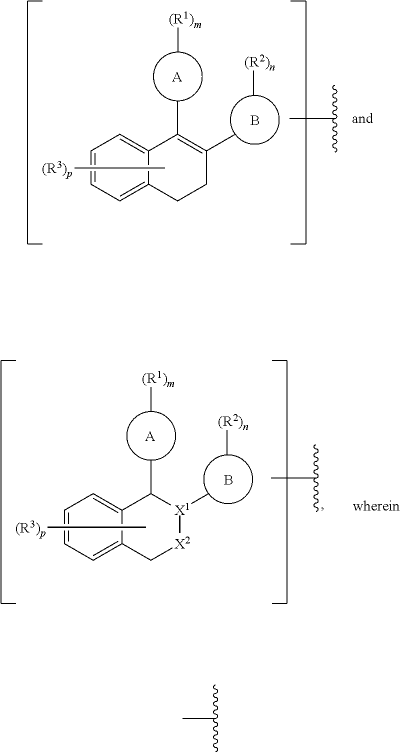

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², RAI, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a bivalent moiety that connects ERBM to LBM;
LBM is a ligase binding moiety;
m is 0, 1, 2, 3, or 4;
n is 0, 1, 2, 3, or 4; and
p is 0, 1, 2, 3, or 4.

In some embodiments, the present disclosure provides a compound of formula I-2:

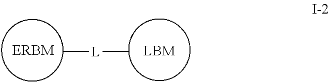

or a pharmaceutically acceptable salt thereof, wherein:
ERBM is selected from

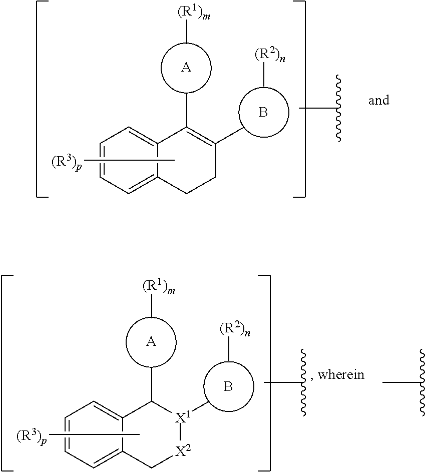

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a bivalent moiety that connects ERBM to LBM;
LBM is selected from

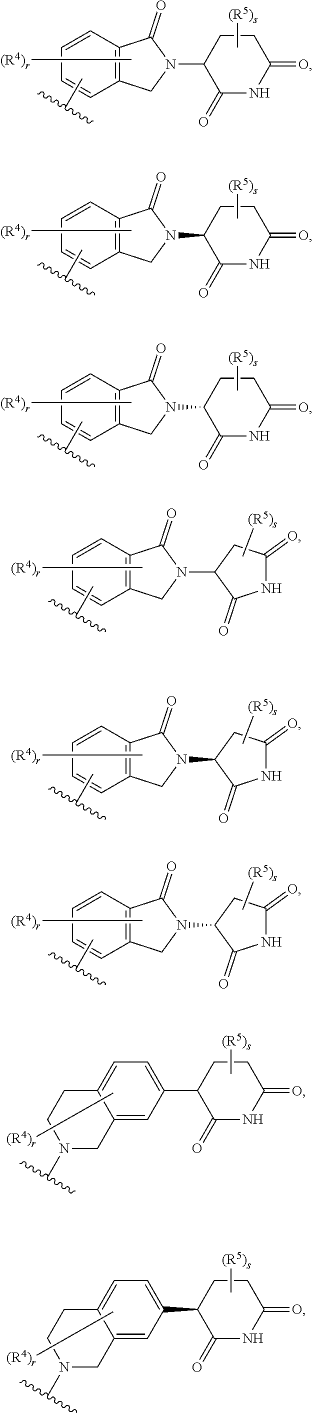

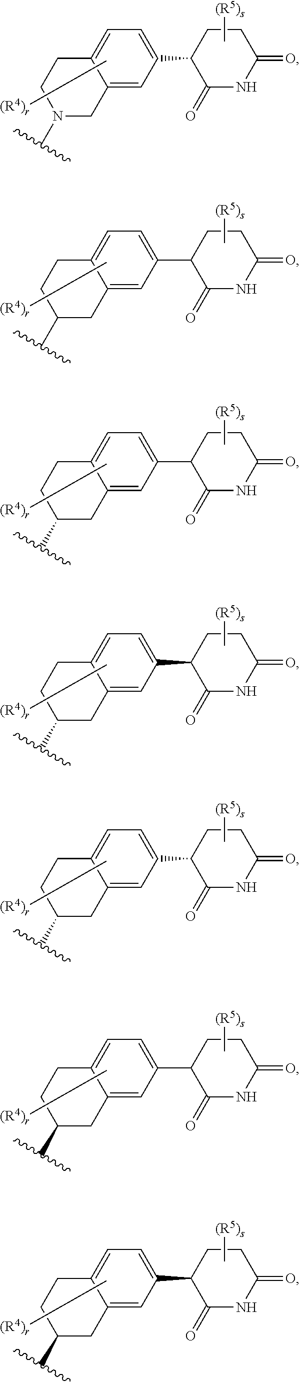

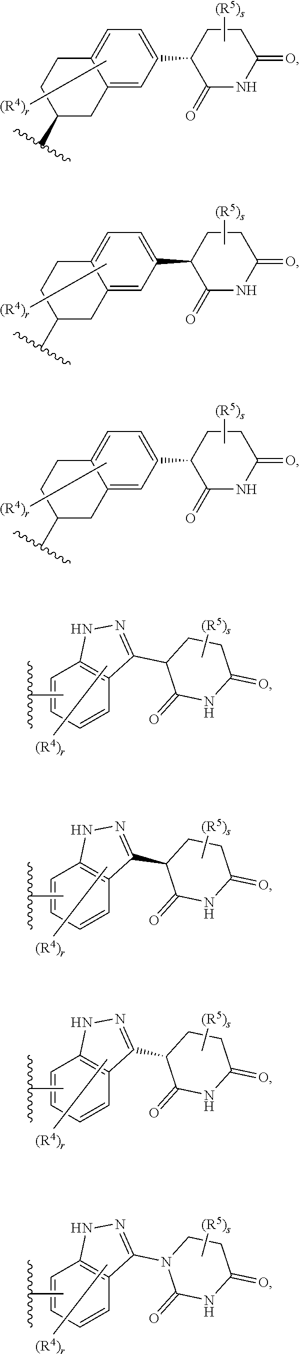

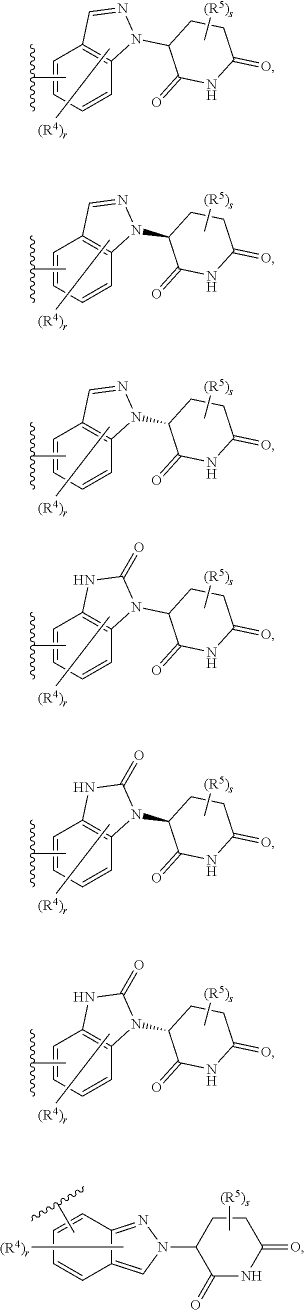

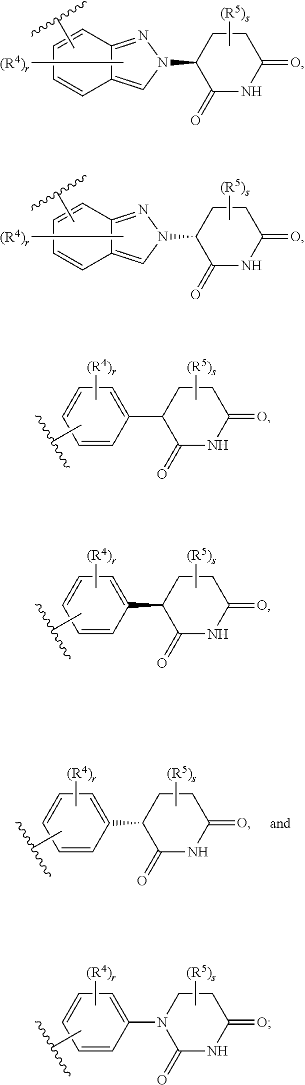

m is 0, 1, 2, 3, or 4;
n is 0, 1, 2, 3, or 4;
p is 0, 1, 2, 3, or 4;
r is 0, 1, 2, 3, or 4; and
s is 0, 1, 2, 3, or 4.

In some embodiments, provided is a compound of formula I-3:

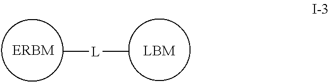

or a pharmaceutically acceptable salt thereof, wherein;
ERBM is selected from

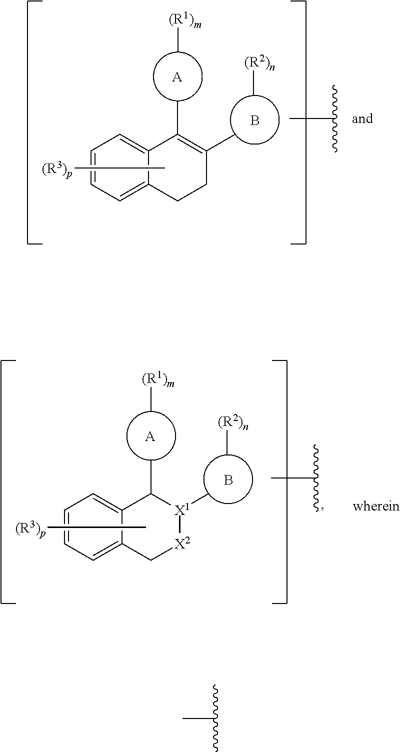

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

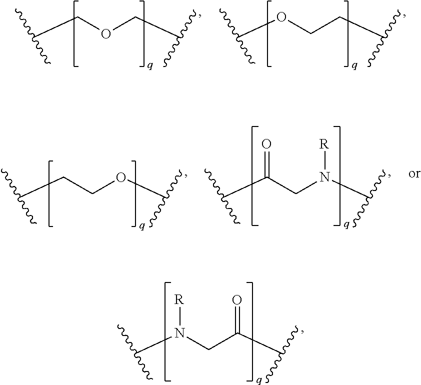

wherein:
each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

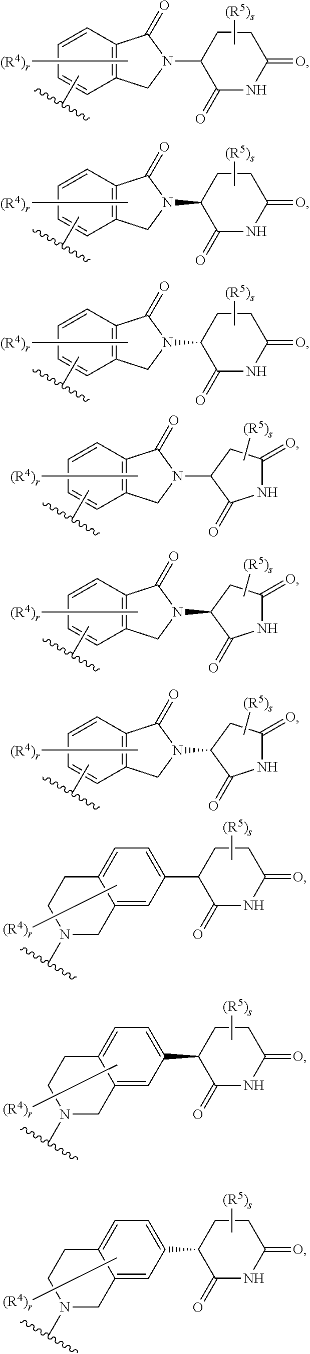

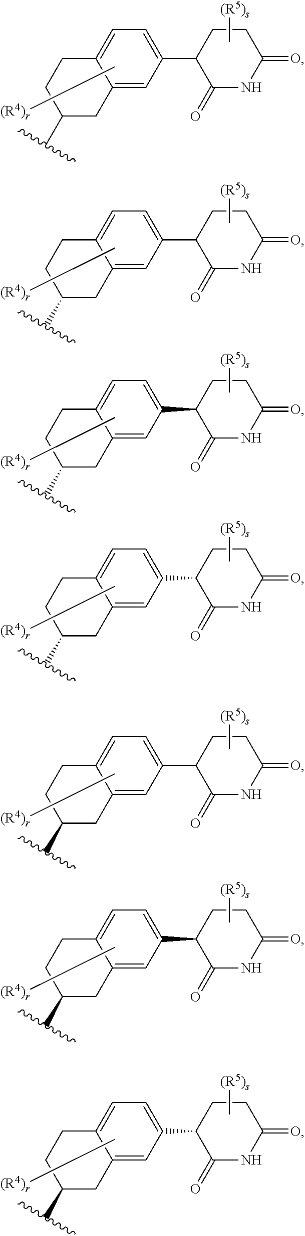

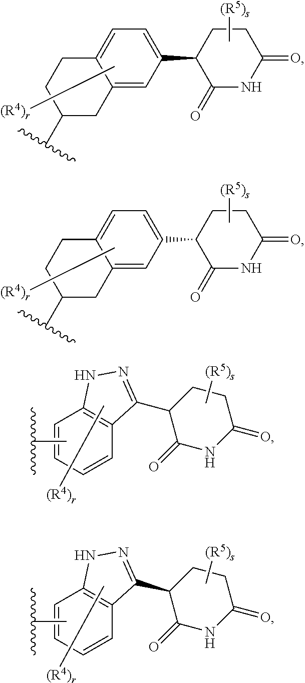

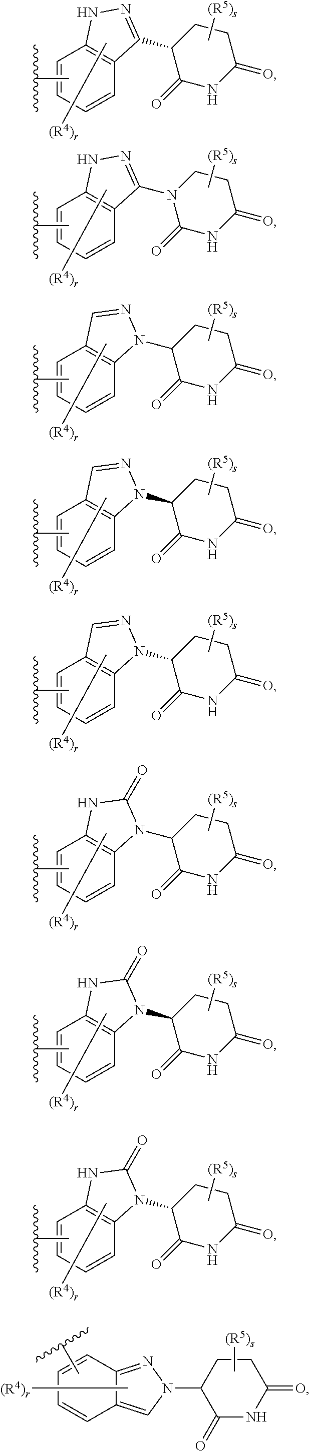

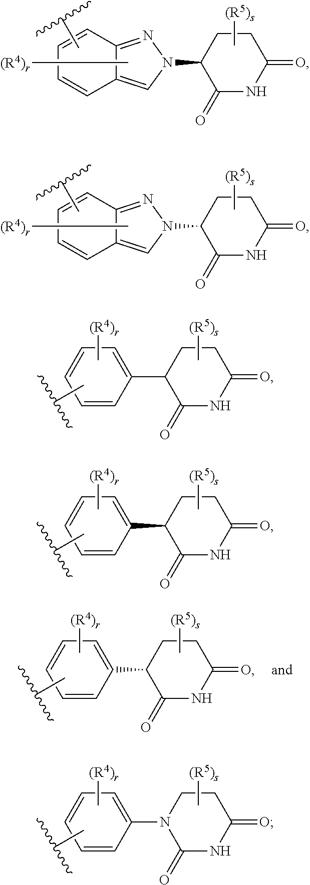

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4.

In an embodiment, provided is a compound of formula I-3′:

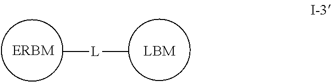

or a pharmaceutically acceptable salt thereof, wherein;
ERBM is selected from

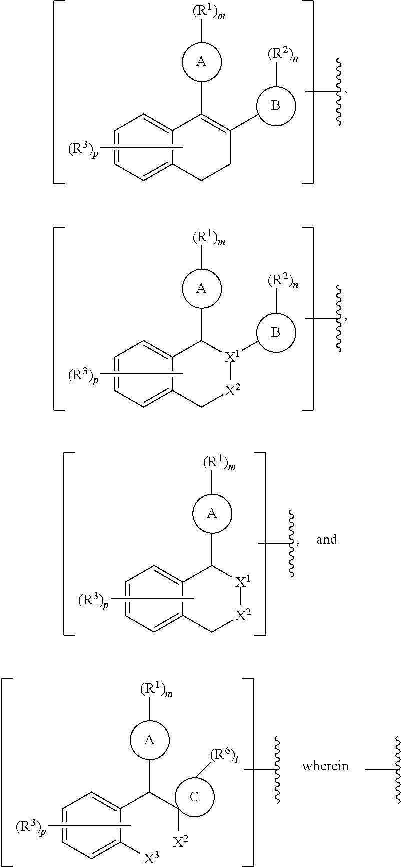

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1}), or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or RR, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;

L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

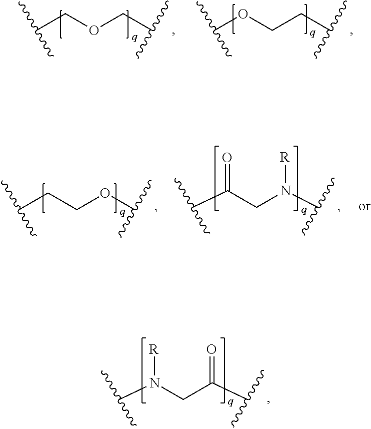

wherein:
each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

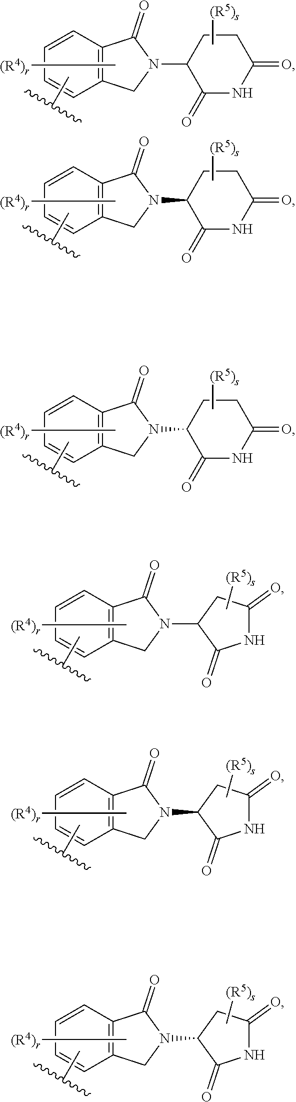

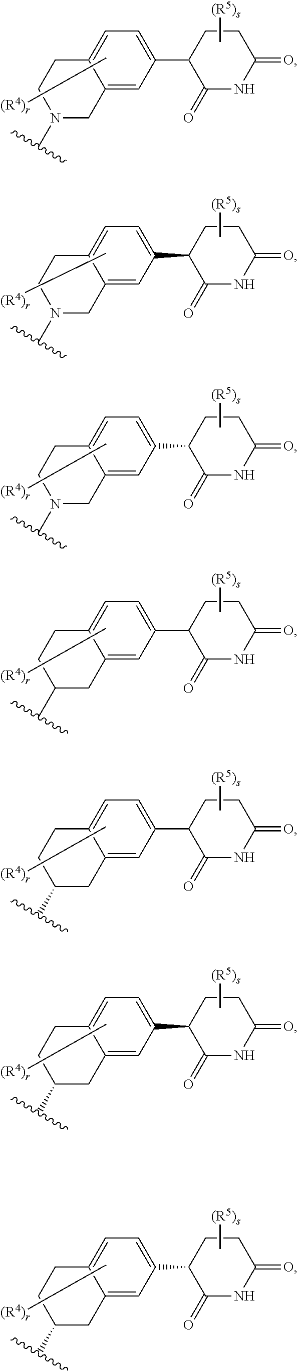

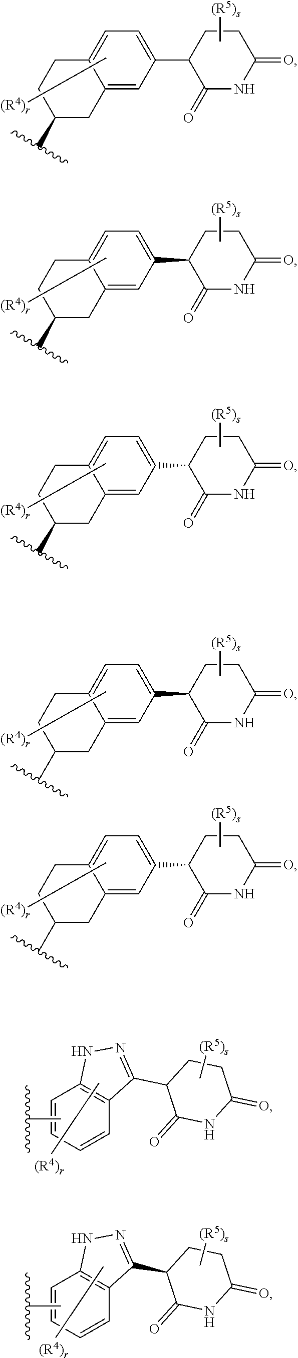

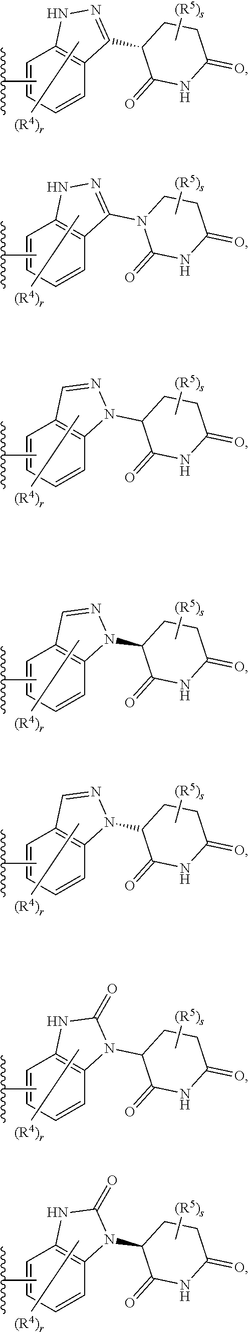

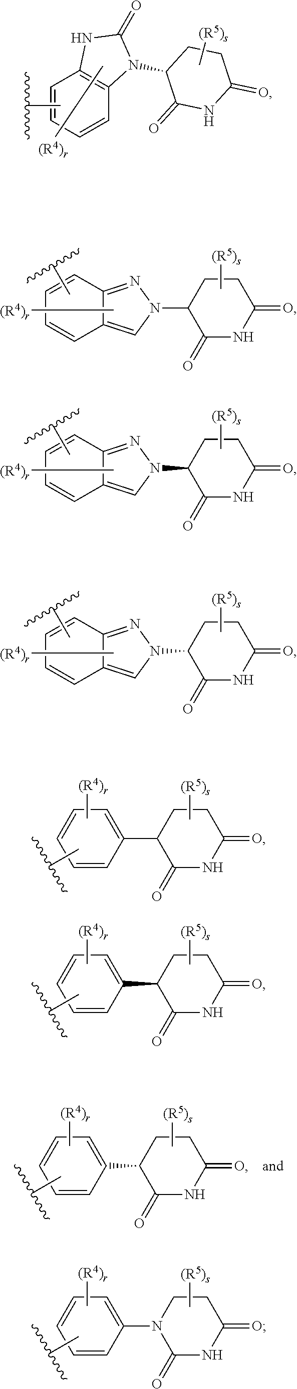

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The terms “carbocyclyl” or “carbocyclic group”, unless otherwise defined, refer to a saturated or partially unsaturated, but not aromatic, 3-10 membered monocyclic or 5-14 membered polycyclic ring system, including bridged, spiro or fused rings, and whose ring system includes exclusively carbon atoms.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl.” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

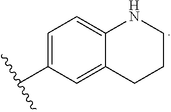 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

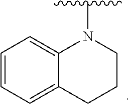 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

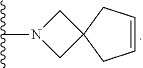 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

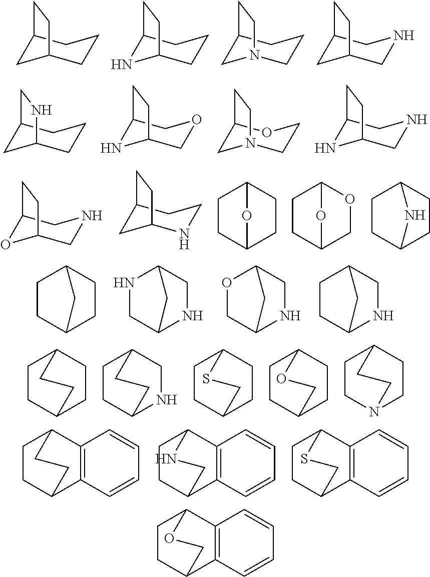

The suffix “ylenyl,” as used herein, refers to a bi-valent radical (i.e., a radical with two attachment points) of the moiety to which “ylenyl” is appended. For example, the term “heterocyclylenyl,” as used herein, refers to a heterocycle with two points of attachment to the rest of the molecule. One or both points of attachment could be through carbon atoms or heteroatoms. In another example, the term “phenylenyl,” as used herein, refers to a divalent form of an optionally substituted phenyl group by itself or in part of another group, and the term “cycloalkylenyl,” as used herein, refers to a divalent form of an optionally substituted cycloalkyl group by itself or part of another group.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R)C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)?OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂—OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₅ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R together with their intervening atoms), are independently halogen, —(CH₂)₀₋₂R^{•}, -(haloR^{•}), —(CH₂)₀₋₂OH, —(CH₂)₀₋₂OR, —(C₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}2, or —NO₂, wherein each R is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having; a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure. Such compounds are useful, for example, as analytical tools, as probes in biological assays, or as therapeutic agents in accordance with the present disclosure. In certain embodiments, for example, a warhead moiety, R^{W}, of a provided compound comprises one or more deuterium atoms.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate sats, and the like.

Salts derived from appropriate bases include alkali metal, alkaline earth metal, ammonium and N⁺(C₁₋₄alkyl)₄ salts. Representative alkali or alkaline earth metal salts include sodium, lithium potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to ERα signaling, or one or more symptoms thereof, prevent the advancement of conditions or symptoms related to afflictions related to ERα signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining; beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to ERα signaling or that may otherwise be relieved by the compounds and/or compositions of the disclosure.

### 3. Description of Exemplary Embodiments

As described above, in some embodiments, the present disclosure provides a compound of formula I-1:

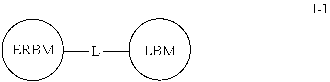

or a pharmaceutically acceptable salt thereof, wherein:
ERBM is selected from

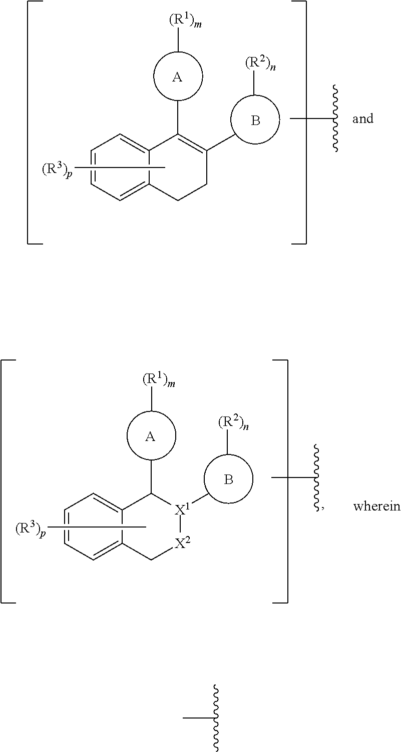

indicates the site of attachment of the -L-LBM moiety to a modifiable carbon, oxygen, nitrogen, or sulfur atom of the ERBM moiety;
X is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R^{A1}, R^{A2} and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)?R, —S(O)₂NR₂, —S(O)₂F, —S(O)R, —S(O)NR—, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a bivalent moiety that connects ERBM to LBM;
LBM is a ligase binding moiety;
m is 0, 1, 2, 3, or 4;
n is 0, 1, 2, 3, or 4; and
p is 0, 1, 2, 3, or 4.

As described above, in some embodiments, the present disclosure provides a compound of formula I-1′:

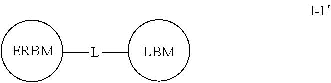

or a pharmaceutically acceptable salt thereof, wherein:
ERBM is selected from

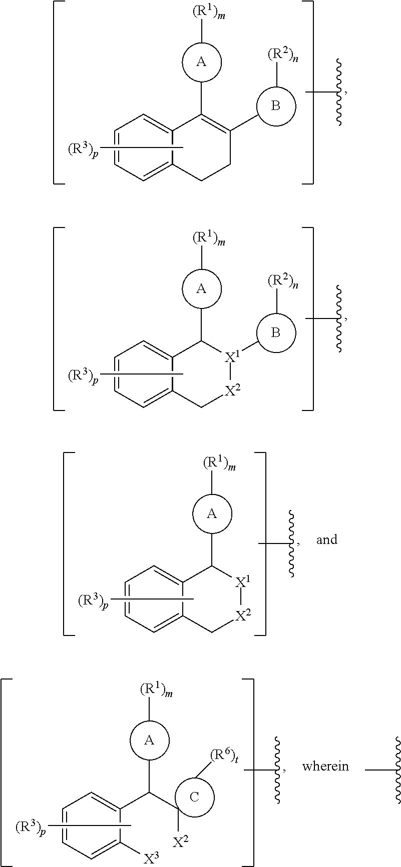

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R₂, R⁶, R^{A1}, R^{A2}R^{A3}, R^{A4} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a bivalent moiety that connects ERBM to LBM;
LBM is a ligase binding moiety;
m is 0, 1, 2, 3, or 4;
n is 0, 1, 2, 3, or 4;
p is 0, 1, 2, 3, or 4; and
t is 0, 1, 2, 3 or 4.

In some embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966; T. Uehara et al. Nature Chemical Biology 2017, 13, 675; WO 2017/176708, US 2017/0281784 (LBM corresponds to the Cereblon-Binding Warhead); WO 2017/161119, US 2021/0177825 (LBM corresponds to the cereblon E3 Ubiquitin Ligase binding moiety CLM); WO 2017/176957, WO 2017/176958, WO 2017/180417, US 2019/0119289, US 2019/0169195, US 2019/0127387, US 2021/0002289 (LBM corresponds to B); WO 2015/160845, WO 2016/197032, WO 2019/199816, US 2015/0291562, US 2016/0058872, US 2018/0228907, US 2020/0155689, US 2020/0155690, US 2022/0089570 (LBM corresponds to ULM); WO 2016/105518, US 2018/0009779, WO 2017/007612, 2018/0134684 (LBM corresponds to Degron); WO 2013/106643, US 2014/0356322 (LBM corresponds to ULM); WO 2002/020740, US 2002/0068063 (LBM corresponds to A); WO 2016/118666, US 2016/0214972 (LBM corresponds to ULM); WO 2016/149668, US 2016/0272639 (LBM corresponds to ULM); WO 2016/169989, US 2018/0118733 (LBM corresponds to IAP binder); WO 2016/197114, US 2018/0147202 (LBM corresponds to ULM); WO 2017/011371, US 2017/0008904 (LBM corresponds to ULM); WO 2017/011590, US 2017/0037004 (LBM corresponds to ULM); WO 2017/079267, US 2017/0121321 (LBM corresponds to ULM); WO 2017/117473, US 2020/0216454 (LBM corresponds to Degron); WO 2017/117474, US 2019/0016703 (LBM corresponds to Degron); WO 2017/197036, US 2019/0076540 (LBM corresponds to Degron); WO 2017/197046, US 2019/0076542 (LBM corresponds to Degron); WO 2017/197051, US 2019/0076539 (LBM corresponds to Degron); WO 2017/197055, US 2019/0076541 (LBM corresponds to Degron); WO 2017/197056 (LBM corresponds to Degron), WO 2022/081928 (LBM corresponds to Degron); WO 2019/060742, US 2021/0238193 (LBM corresponds to UBM); WO 2019/140380, US 2021/0002296 (LBM corresponds to UBM); WO 2020/010177, US 2020/0010468 (LBM corresponds to CRBN ligand); WO 2020/010227 (LBM corresponds to UBM); and WO 2021/011634 (LBM corresponds to UBM); the entirety of each of which is herein incorporated by reference.

In some embodiments, L is a bivalent moiety that connects ERBM to LBM. Such L moieties are well known to one of ordinary skill in the art and include those described in M. Toure, C. M. Crews, Angew. Chem. Int. Ed. 2016, 55, 1966; T. Uehara et al. Nature Chemical Biology 2017, 13, 675; WO 2017/176708, US 2017/0281784 (L corresponds to the connector C); WO 2017/161119, US 2021/0177825 (L corresponds to the linker L); WO 2017/176957, WO 2017/176958, WO 2017/180417, US 2019/0119289, US 2019/0169195, US 2019/0127387, US 2021/0002289 (L corresponds to L); WO 2015/160845, WO 2016/197032, WO 2019/199816, US 2015/0291562, US 2016/0058872, US 2018/0228907, US 2020/0155689, US 2020/0155690, US 2022/0089570 (L corresponds to the chemical linker L); WO 2016/105518, US 2018/0009779, WO 2017/007612, 2018/0134684 (L corresponds to Linker); WO 2013/106643, US 2014/0356322 (L corresponds to linker group L); WO 2016/118666, US 2016/0214972 (L corresponds to L); WO 2016/149668, US 2016/0272639 (L corresponds to Linker); WO 2016/169989, US 2018/0118733 (L corresponds to L); WO 2016/197114, US 2018/0147202 (L corresponds to L); WO 2017/011371, US 2017/0008904 (L corresponds to L); WO 2017/011590, US 2017/0037004 (L corresponds to L); WO 2017/079267, US 2017/0121321 (L corresponds to L); WO 2017/117473, US 2020/0216454 (L corresponds to Linker); WO 2017/117474, US 2019/0016703 (L corresponds to Linker); WO 2017/197036, US 2019/0076540 (L corresponds to Linker); WO 2017/197046, US 2019/0076542 (L corresponds to Linker); WO 2017/197051, US 2019/0076539 (L corresponds to Linker); WO 2017/197055, US 2019/0076541 (L corresponds to Linker); WO 2017/197056 (LBM corresponds to Degron); WO 2022/081928 (L corresponds to Linker and/or Spacer); WO 2019/060742, US 2021/0238193 (L corresponds to L); WO 2019/140380, US 2021/0002296 (L corresponds to L); WO 2020/010227 (L corresponds to L); and WO 2021/011634 (L corresponds to L); the entirety of each of which is herein incorporated by reference.

In some embodiments, the present disclosure provides a compound of formula I-2:

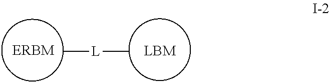

or a pharmaceutically acceptable salt thereof, wherein:
ERBM is selected from

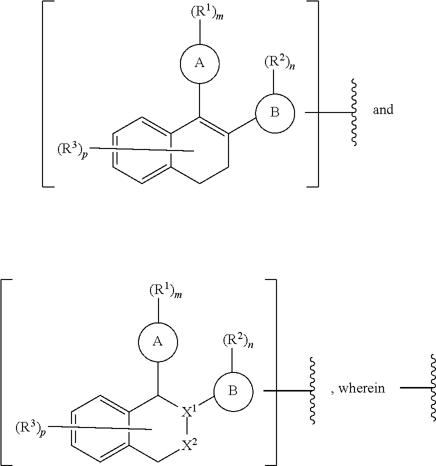

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R₂, R₄, R₅, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently 1,1′-biphenyl; phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a bivalent moiety that connects ERBM to LBM;
LBM is selected from

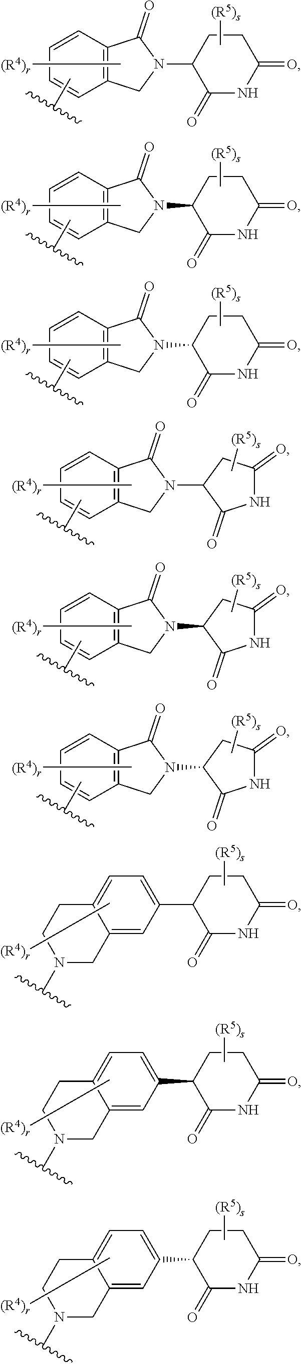

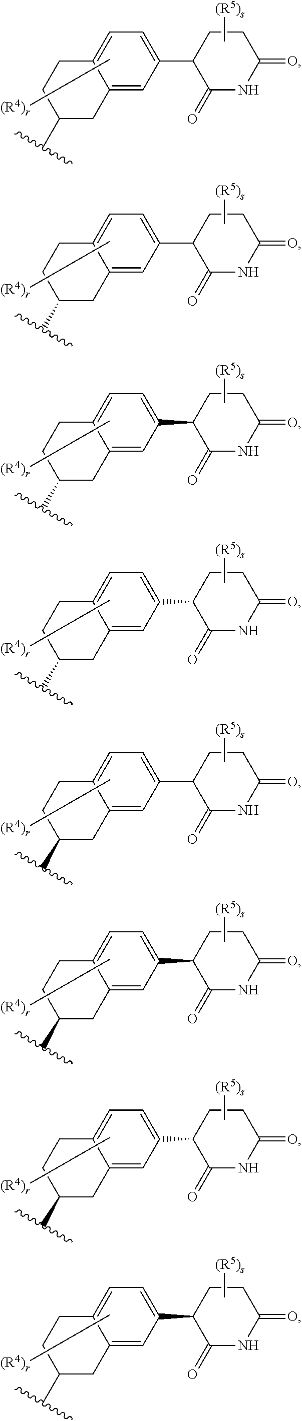

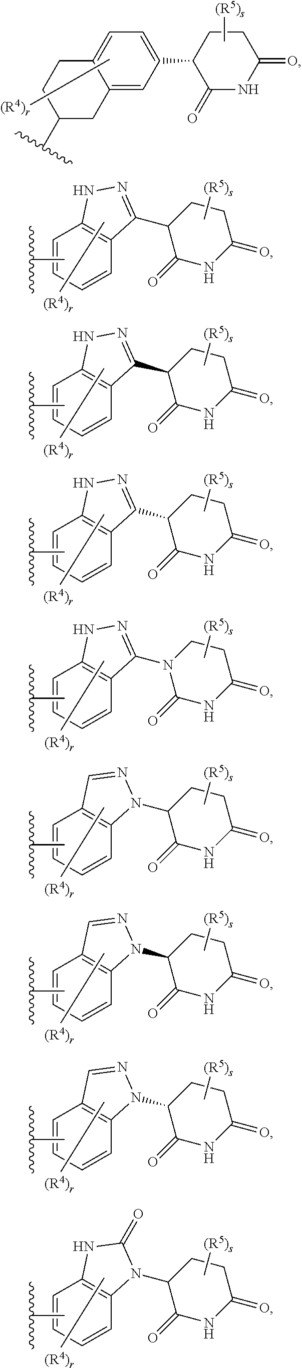

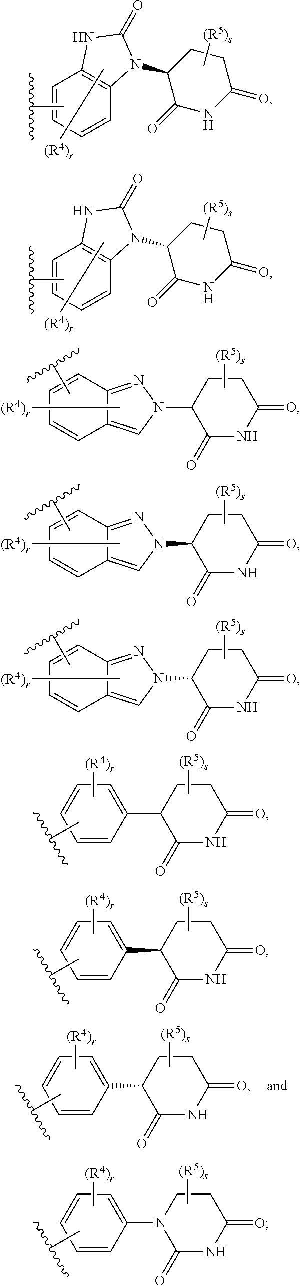

m is 0, 1, 2, 3, or 4;
n is 0, 1, 2, 3, or 4;
p is 0, 1, 2, 3, or 4;
r is 0, 1, 2, 3, or 4; and
s is 0, 1, 2, 3, or 4.

In some embodiments, the present disclosure provides a compound of formula I-2′:

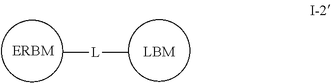

or a pharmaceutically acceptable salt thereof, wherein:
ERBM is selected from

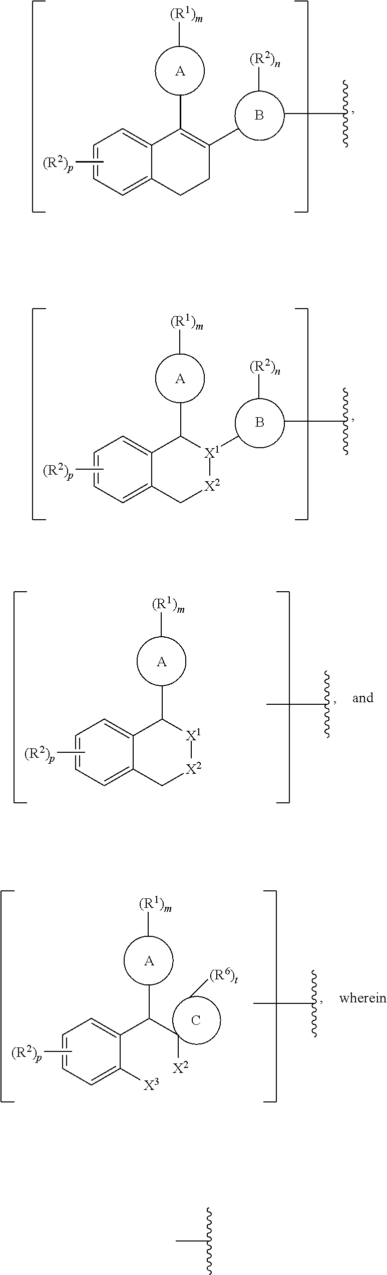

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
Provided that X¹ and X² or X² and X³ are not both heteroatoms;
each instance of R¹, R₂, R₄, R₅, R⁶, R^{A1}, R^{A2}, R^{A3}, R^{A4} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a bivalent moiety that connects ERBM to LBM;
LBM is selected from

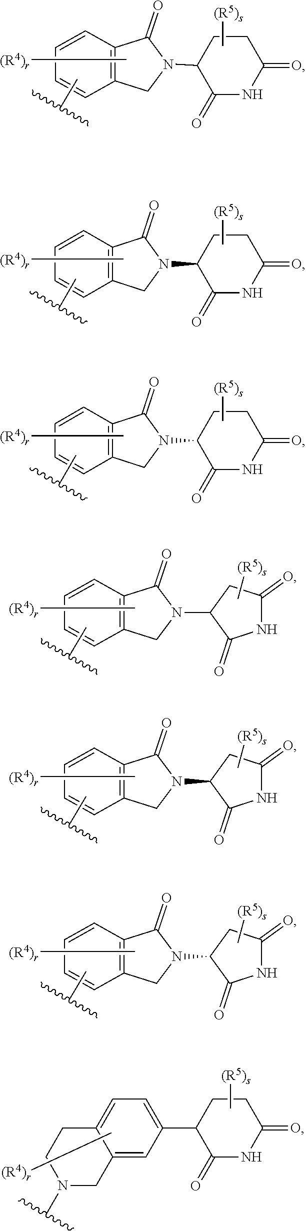

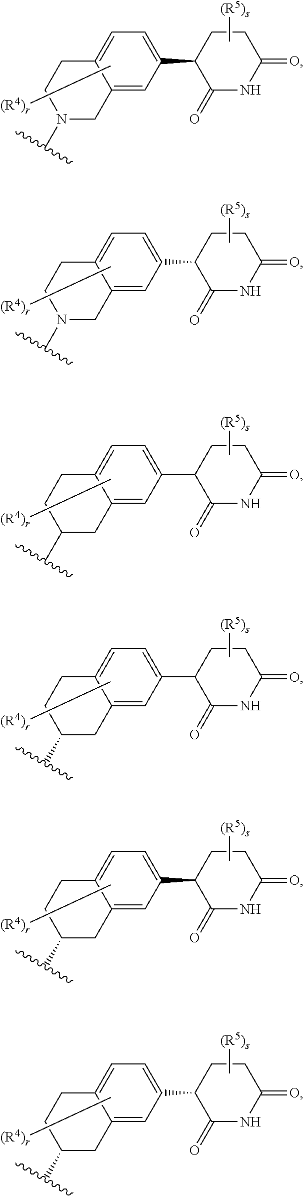

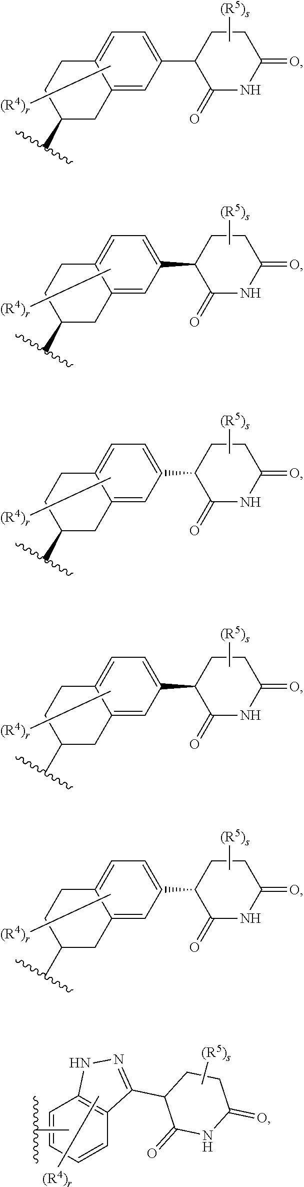

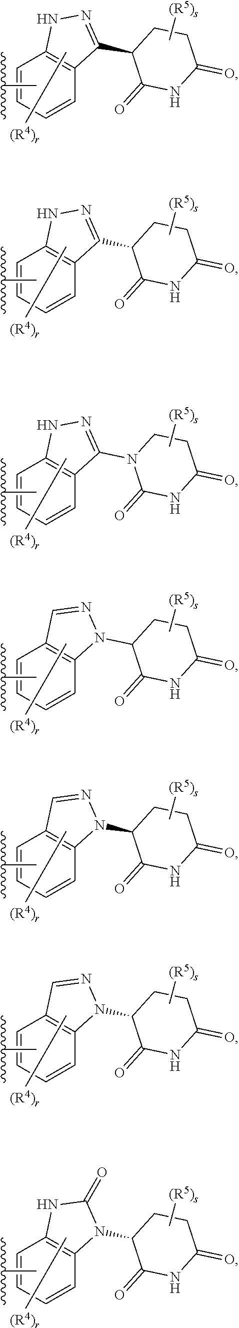

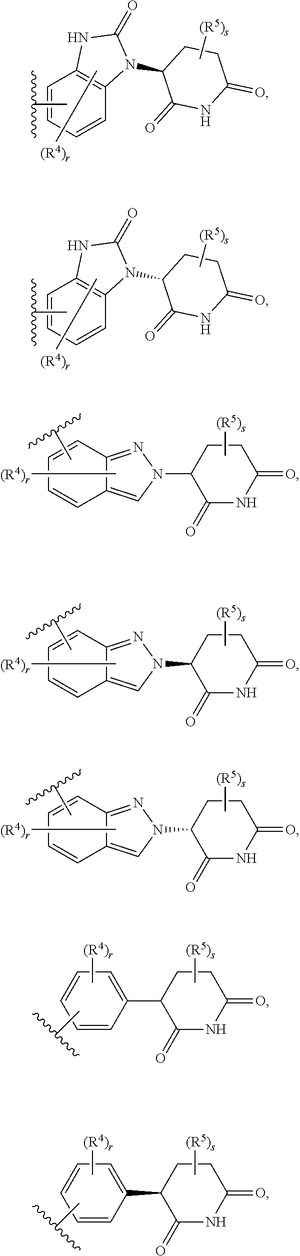

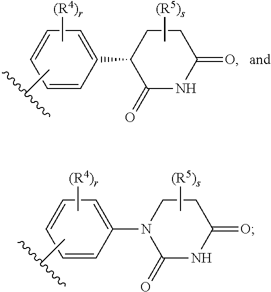

m is 0, 1, 2, 3, or 4;
n is 0, 1, 2, 3, or 4;
p is 0, 1, 2, 3, or 4;
r is 0, 1, 2, 3, or 4;
s is 0, 1, 2, 3, or 4; and
t is 0, 1, 2, 3, or 4.

In some embodiments, the present disclosure provides a compound of formula I-3:

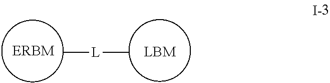

or a pharmaceutically acceptable salt thereof, wherein:
ERBM is selected from

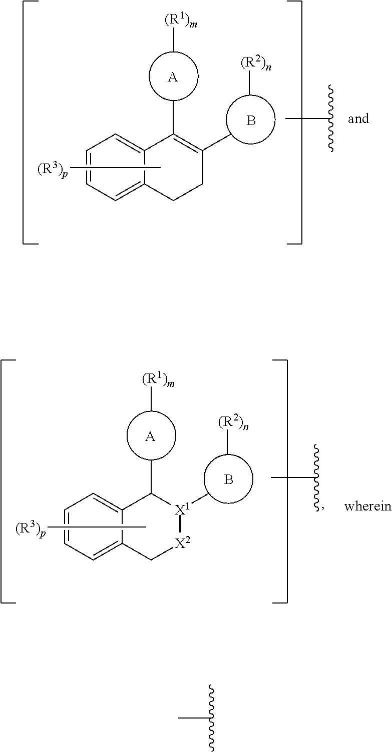

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R₂, R₄, R₅, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂ or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from independently selected from nitrogen, oxygen, and sulfur; a 3-7 membered saturated or partially bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

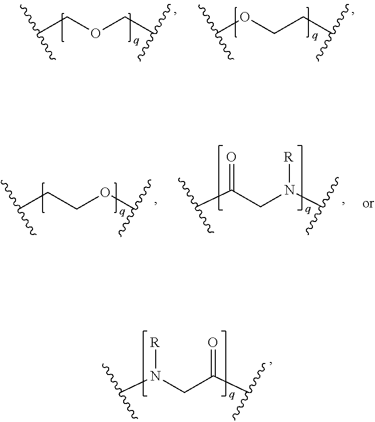

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

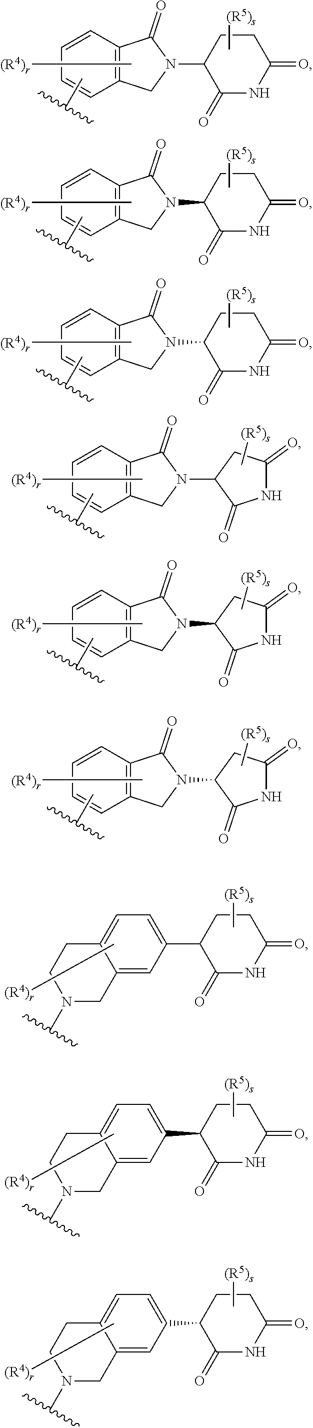

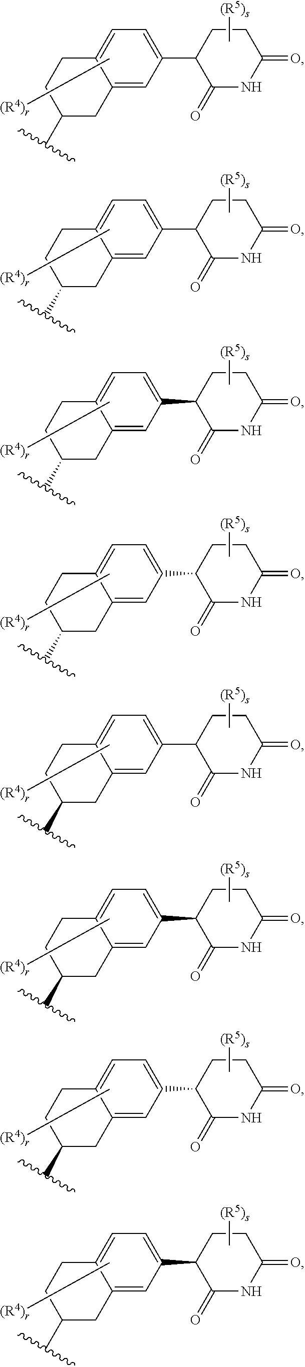

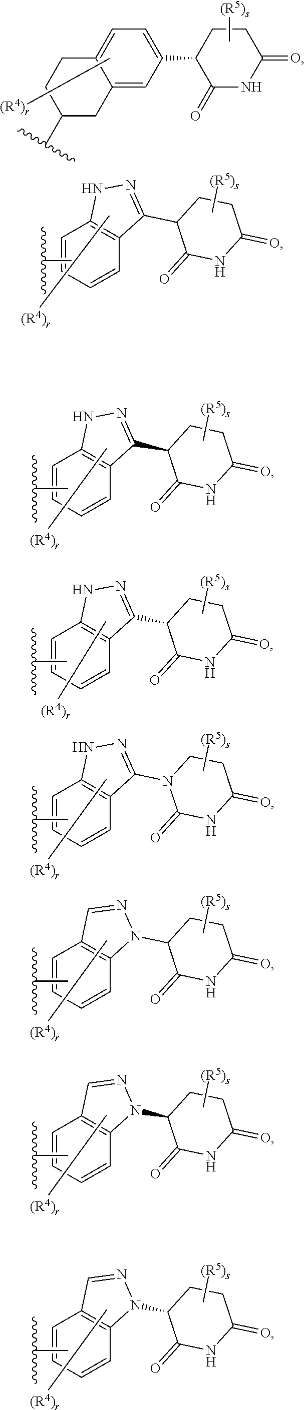

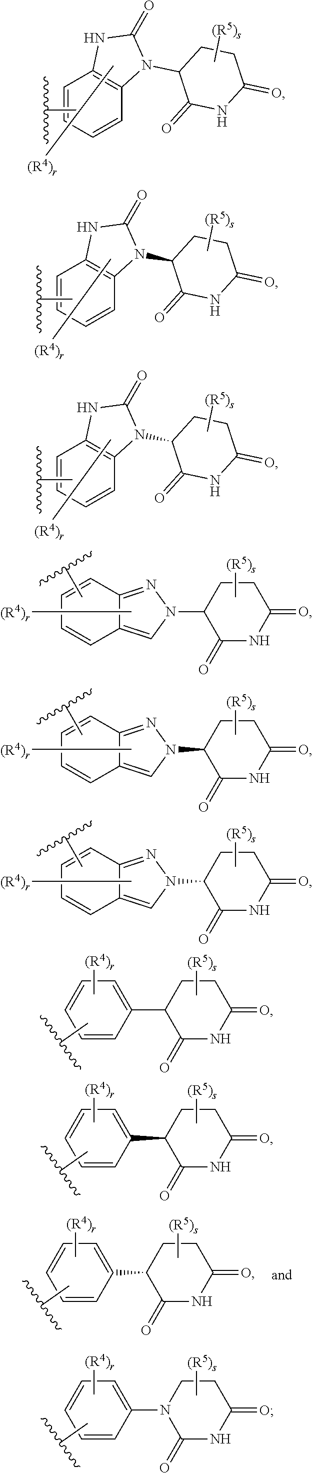

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4.

In an embodiment, provided is a compound of formula I-3′:

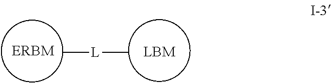

or a pharmaceutically acceptable salt thereof, wherein;
ERBM is selected from

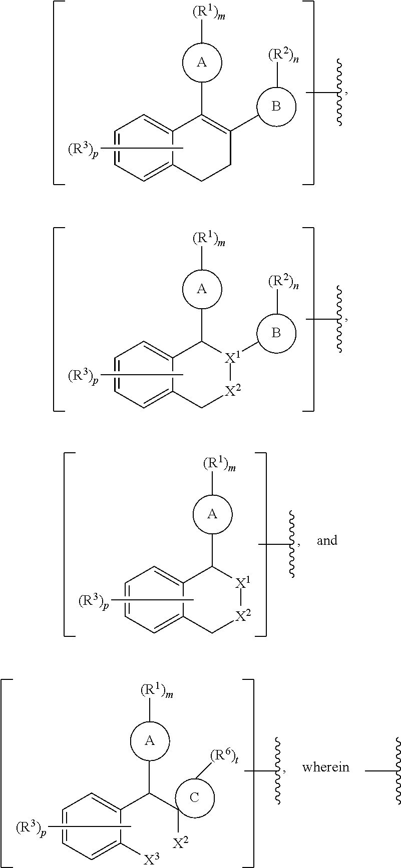

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R₂, R₄, R₅, R⁶, R^{A1}, R^{A2}, R^{A3}, R^{A4}, and R^{A5} is independently R^{A} or RR, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR) z, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

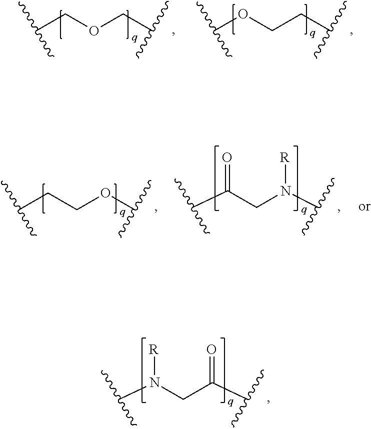

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

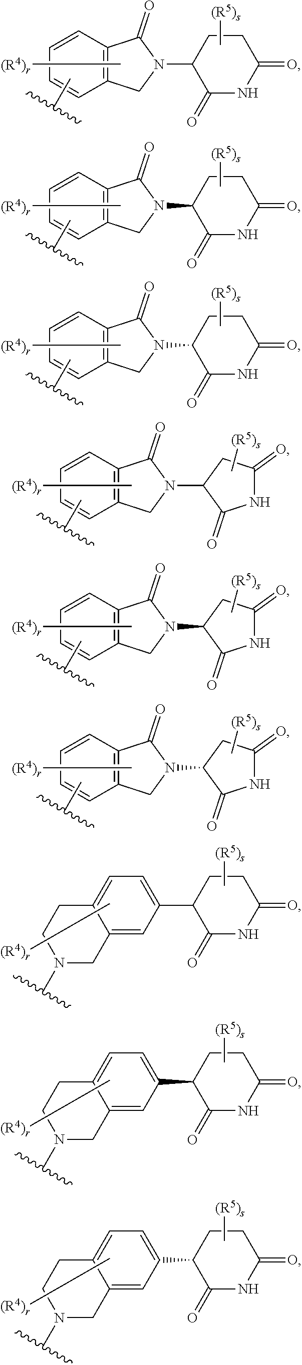

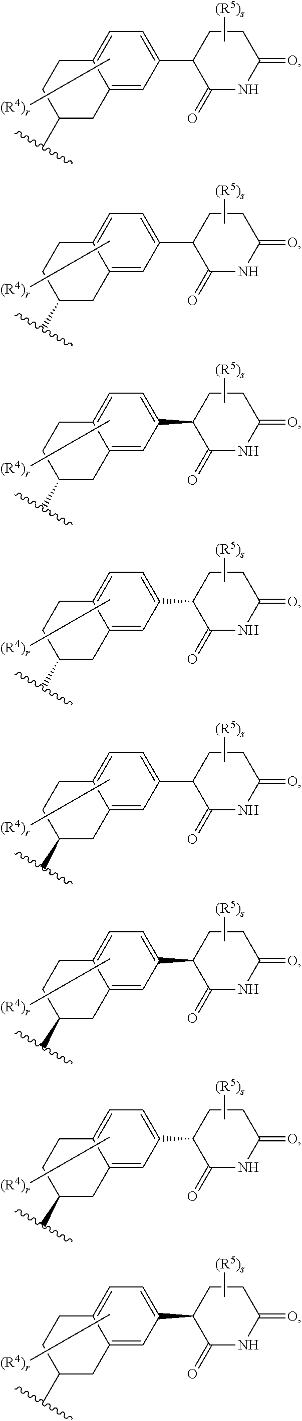

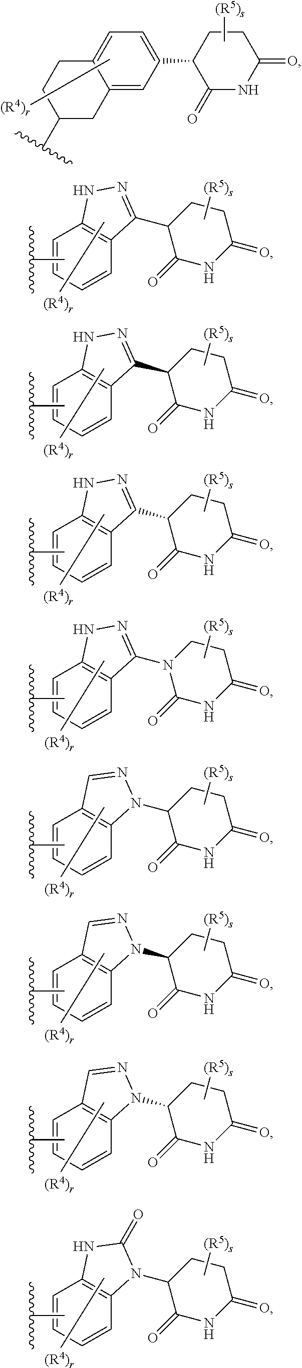

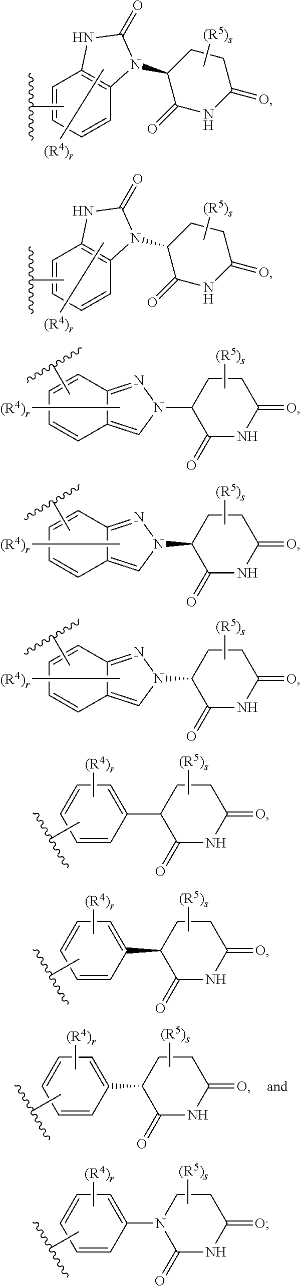

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

In some embodiments, the present disclosure provides a compound of formula I-1, I-1′, I-2, I-2′, I-3 or I-3′ or any subgenera thereof, provided that when ERBM is

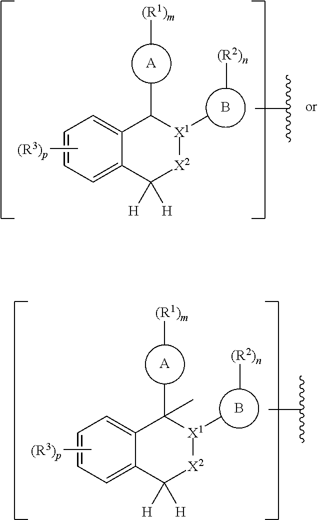

wherein X¹ is CH or N,
   X² is CH₂;
   Ring B is phenyl;
   Ring A is phenyl or a 6 membered monocyclic heteroaryl ring having 1-3 nitrogen heteroatoms;
   at least one R³ is —OH or —OMe;
   p is 1, 2, or 3;
   m is 0, 1 or 2;
   R¹ is selected from —F and —Cl;
   n is 0, 1 or 2; and
   R² is selected from —OH, -Me, —OMe, —F, —Br, —CF₃ and —ⁱPr;
then LBM is not

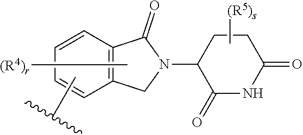

or a stereoisomer thereof.

In some embodiments, the present disclosure provides a compound of formula I-1, I-1′, I-2, I-2′, I-3 or I-3′ or any subgenera thereof, provided that when ERBM is

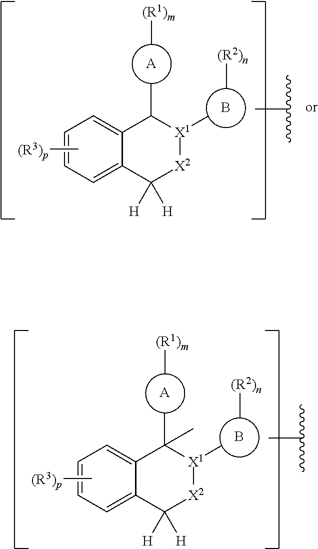

wherein X¹ is CH or N,
   X² is CH₂;
   Ring B is phenyl;
   Ring A is phenyl or a 6 membered monocyclic heteroaryl ring having 1-3 nitrogen heteroatoms;
then R³ is not-OH or —OMe.

In some embodiments, the present disclosure provides a compound of formula I-1, I-1′, I-2, I-2′, I-3 or I-3′ or any subgenera thereof, provided that: when ERBM is

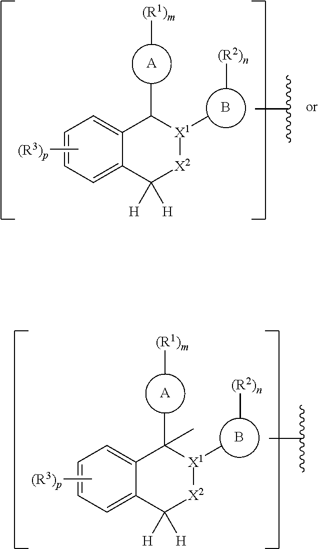

wherein X¹ is CH or N,
   X² is CH₂;
   Ring B is phenyl;
then Ring A is not phenyl or a 6 membered monocyclic heteroaryl ring having 1-3 nitrogen heteroatoms.

In some embodiments, the present disclosure provides a compound of formula I-1, I-1′, I-2, I-2′, I-3 or I-3′, provided that Ring A is other than phenyl or a 6 membered monocyclic heteroaryl ring having 1-3 nitrogen heteroatoms when ERBM is

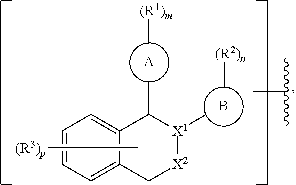

X¹ is CH or N, X² is CH₂ and Ring B is phenyl.

As defined generally above, ERBM is an ERα binding moiety capable of binding to ERα.

As defined generally above, ERBM IS selected from

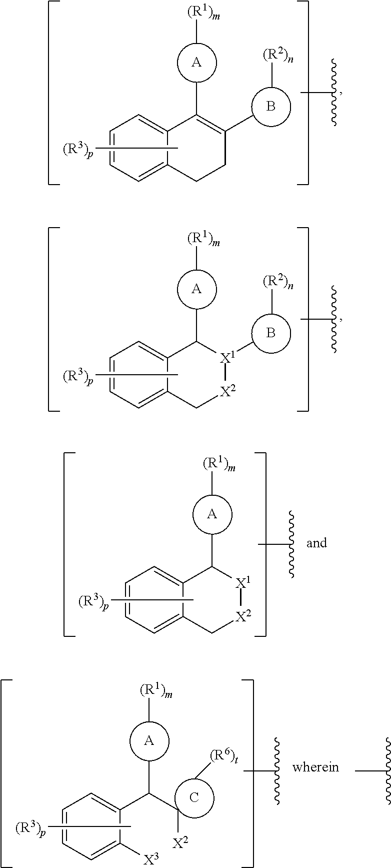

indicates the site of attachment of the -L-LBM moiety to a modifiable carbon, oxygen, nitrogen, or sulfur atom of the ERBM moiety and wherein X¹, X², X³, Ring A, Ring B, Ring C, R¹, R², R³, R⁶, m, n, p and t are as defined in any of the embodiments described herein.

In some embodiments, ERBM is

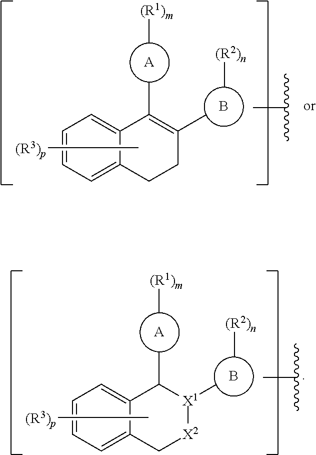

In some embodiments, ERBM is

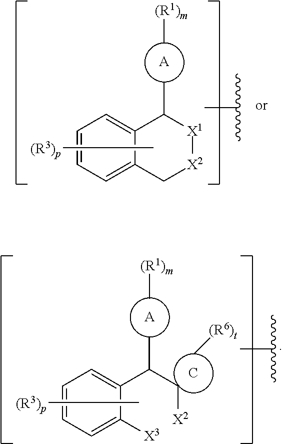

In some embodiments, ERBM is

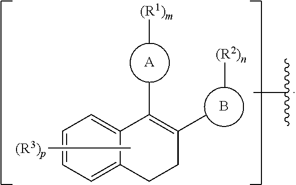

In some embodiments, ERBM is

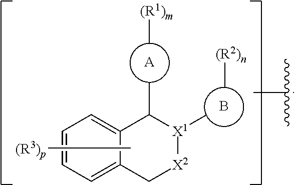

In some embodiments, ERBM is

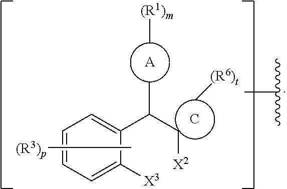

In some embodiments, ERBM is

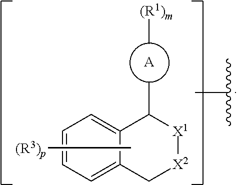

In some embodiments, ERBM is selected from the group consisting of

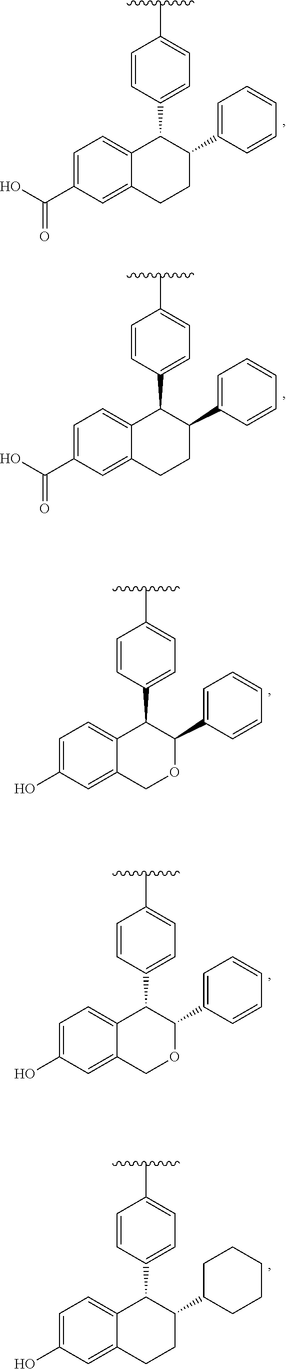 [SMILES: *c1ccc([C@H]2c3ccc(C(=O)O)cc3CC[C@H]2c2ccccc2)cc1 ; *c1ccc([C@@H]2c3ccc(C(=O)O)cc3CC[C@@H]2c2ccccc2)cc1 ; *c1ccc([C@@H]2c3ccc(O)cc3CO[C@@H]2c2ccccc2)cc1 ; *c1ccc([C@H]2c3ccc(O)cc3CO[C@H]2c2ccccc2)cc1 ; *c1ccc([C@H]2c3ccc(O)cc3CC[C@H]2C2CCCCC2)cc1]

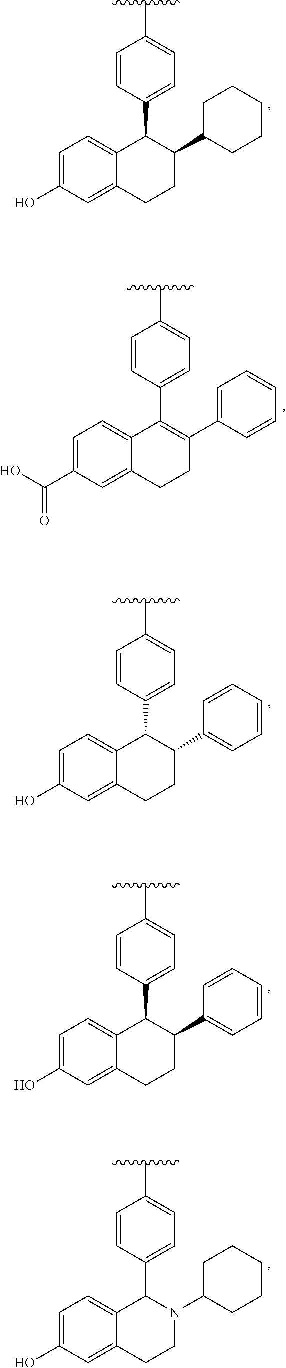 [SMILES: *c1ccc([C@@H]2c3ccc(O)cc3CC[C@@H]2C2CCCCC2)cc1 ; *c1ccc(C2=C(c3ccccc3)CCc3cc(C(=O)O)ccc32)cc1 ; *c1ccc([C@H]2c3ccc(O)cc3CC[C@H]2c2ccccc2)cc1 ; *c1ccc([C@@H]2c3ccc(O)cc3CC[C@@H]2c2ccccc2)cc1 ; *c1ccc(C2c3ccc(O)cc3CCN2C2CCCCC2)cc1]

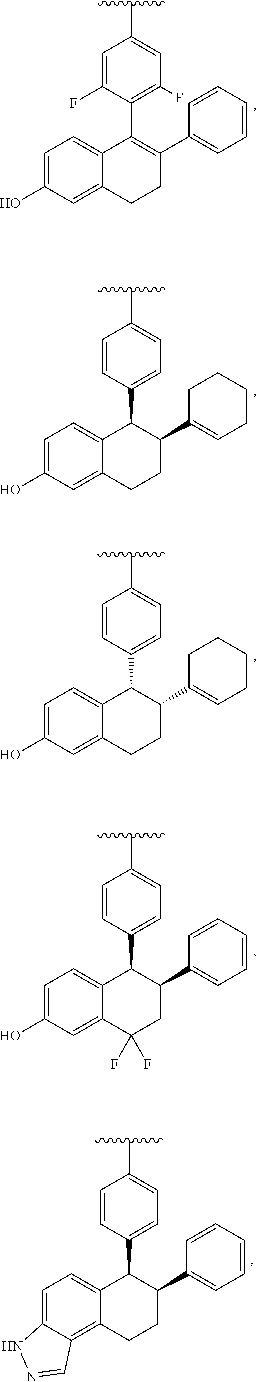 [SMILES: *c1cc(F)c(C2=C(c3ccccc3)CCc3cc(O)ccc32)c(F)c1 ; *c1ccc([C@@H]2c3ccc(O)cc3CC[C@@H]2C2=CCCCC2)cc1 ; *c1ccc([C@H]2c3ccc(O)cc3CC[C@H]2C2=CCCCC2)cc1 ; *c1ccc([C@@H]2c3ccc(O)cc3C(F)(F)C[C@@H]2c2ccccc2)cc1 ; *c1ccc([C@@H]2c3ccc4[nH]ncc4c3CC[C@@H]2c2ccccc2)cc1]

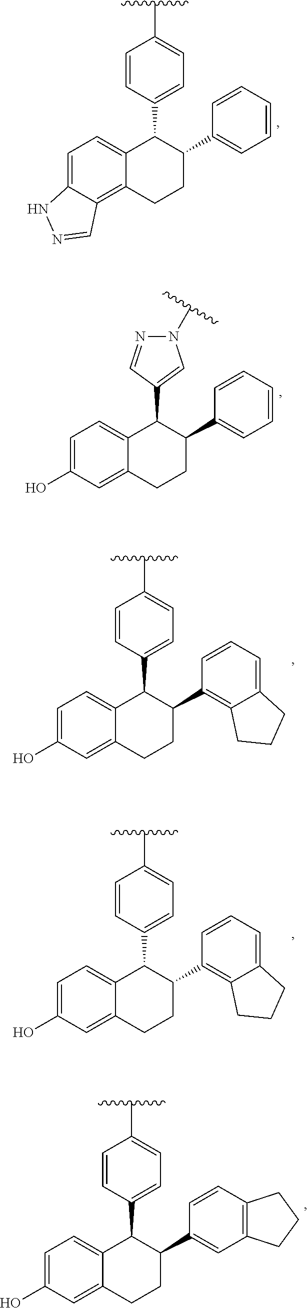 [SMILES: *c1ccc([C@H]2c3ccc4[nH]ncc4c3CC[C@H]2c2ccccc2)cc1 ; *n1cc([C@@H]2c3ccc(O)cc3CC[C@@H]2c2ccccc2)cn1 ; *c1ccc([C@@H]2c3ccc(O)cc3CC[C@@H]2c2cccc3c2CCC3)cc1 ; *c1ccc([C@H]2c3ccc(O)cc3CC[C@H]2c2cccc3c2CCC3)cc1 ; *c1ccc([C@@H]2c3ccc(O)cc3CC[C@@H]2c2ccc3c(c2)CCC3)cc1]

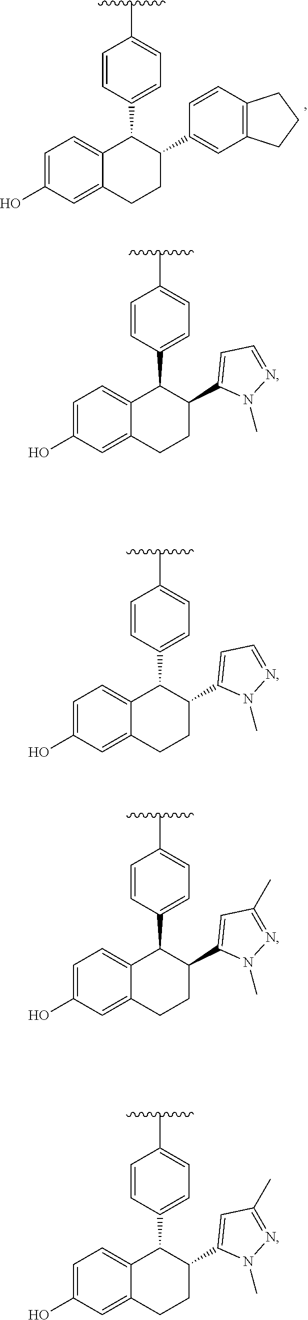 [SMILES: *c1ccc([C@H]2c3ccc(O)cc3CC[C@H]2c2ccc3c(c2)CCC3)cc1 ; *c1ccc([C@@H]2c3ccc(O)cc3CC[C@@H]2c2ccnn2C)cc1 ; *c1ccc([C@H]2c3ccc(O)cc3CC[C@H]2c2ccnn2C)cc1 ; *c1ccc([C@@H]2c3ccc(O)cc3CC[C@@H]2c2cc(C)nn2C)cc1 ; *c1ccc([C@H]2c3ccc(O)cc3CC[C@H]2c2cc(C)nn2C)cc1]

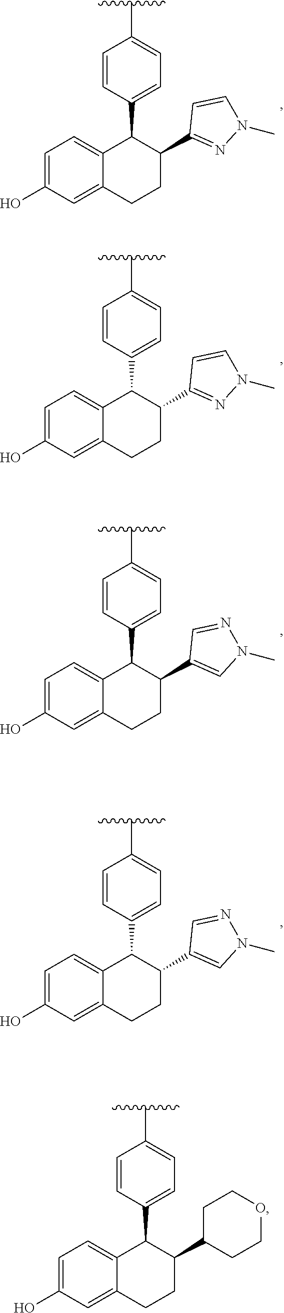 [SMILES: *c1ccc([C@@H]2c3ccc(O)cc3CC[C@@H]2c2ccn(C)n2)cc1 ; *c1ccc([C@H]2c3ccc(O)cc3CC[C@H]2c2ccn(C)n2)cc1 ; *c1ccc([C@@H]2c3ccc(O)cc3CC[C@@H]2c2cnn(C)c2)cc1 ; *c1ccc([C@H]2c3ccc(O)cc3CC[C@H]2c2cnn(C)c2)cc1 ; *c1ccc([C@@H]2c3ccc(O)cc3CC[C@@H]2C2CCOCC2)cc1]

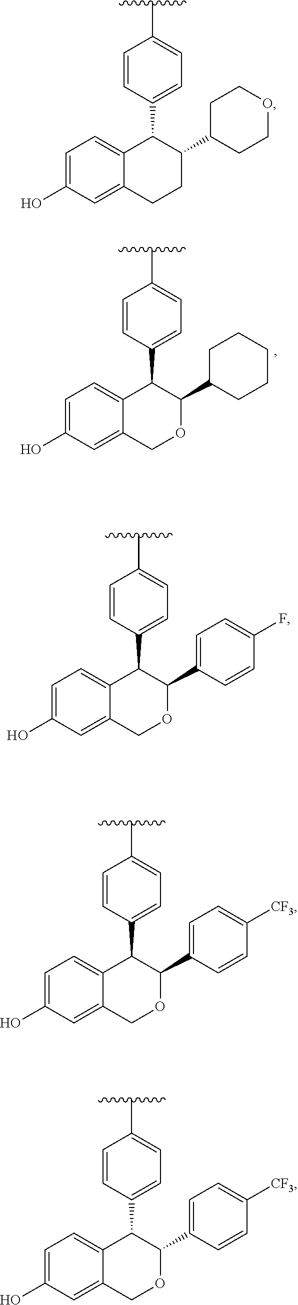

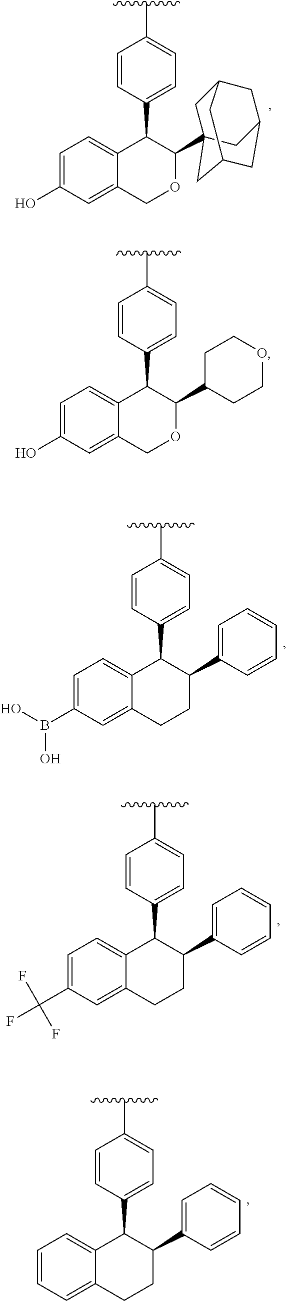 [SMILES: *c1ccc([C@@H]2c3ccc(O)cc3CO[C@@H]2C23CC4CC(CC(C4)C2)C3)cc1 ; *c1ccc([C@@H]2c3ccc(O)cc3CO[C@@H]2C2CCOCC2)cc1 ; *c1ccc([C@@H]2c3ccc(B(O)O)cc3CC[C@@H]2c2ccccc2)cc1 ; *c1ccc([C@@H]2c3ccc(C(F)(F)F)cc3CC[C@@H]2c2ccccc2)cc1 ; *c1ccc([C@@H]2c3ccccc3CC[C@@H]2c2ccccc2)cc1]

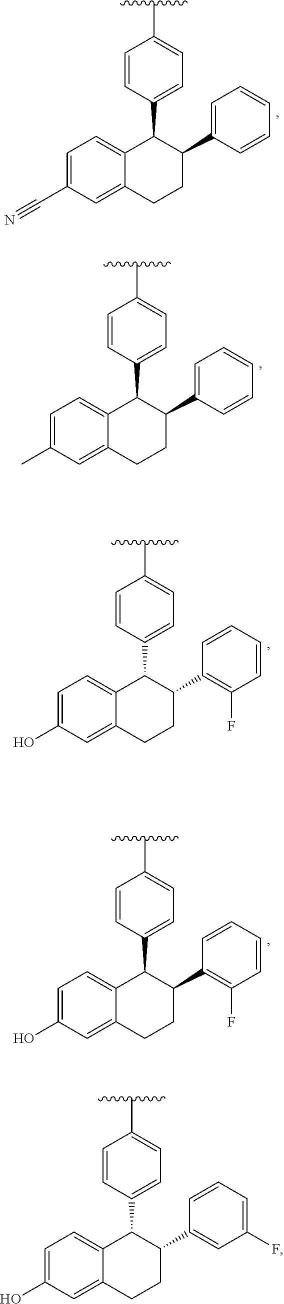 [SMILES: *c1ccc([C@@H]2c3ccc(C#N)cc3CC[C@@H]2c2ccccc2)cc1 ; *c1ccc([C@@H]2c3ccc(C)cc3CC[C@@H]2c2ccccc2)cc1 ; *c1ccc([C@H]2c3ccc(O)cc3CC[C@H]2c2ccccc2F)cc1 ; *c1ccc([C@@H]2c3ccc(O)cc3CC[C@@H]2c2ccccc2F)cc1 ; *c1ccc([C@H]2c3ccc(O)cc3CC[C@H]2c2cccc(F)c2)cc1]

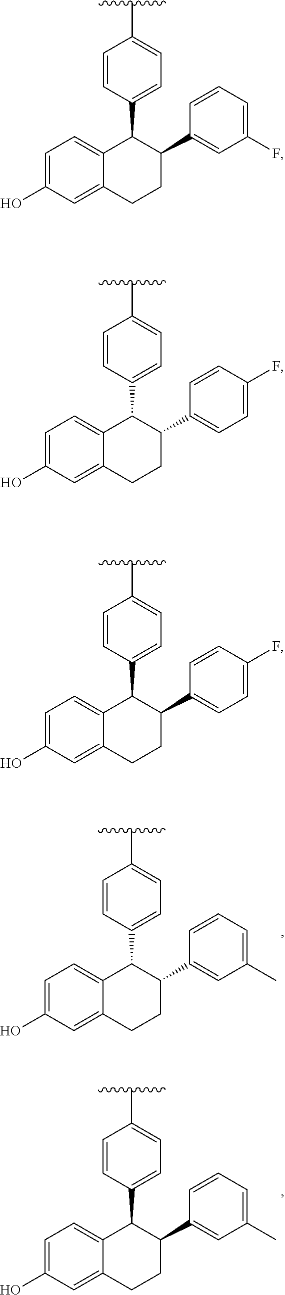 [SMILES: *c1ccc([C@@H]2c3ccc(O)cc3CC[C@@H]2c2cccc(F)c2)cc1 ; *c1ccc([C@H]2c3ccc(O)cc3CC[C@H]2c2ccc(F)cc2)cc1 ; *c1ccc([C@@H]2c3ccc(O)cc3CC[C@@H]2c2ccc(F)cc2)cc1 ; *c1ccc([C@H]2c3ccc(O)cc3CC[C@H]2c2cccc(C)c2)cc1 ; *c1ccc([C@@H]2c3ccc(O)cc3CC[C@@H]2c2cccc(C)c2)cc1]

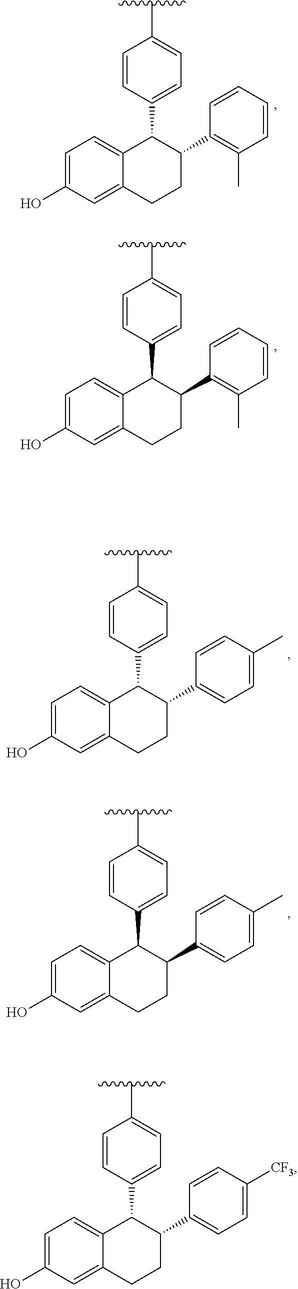

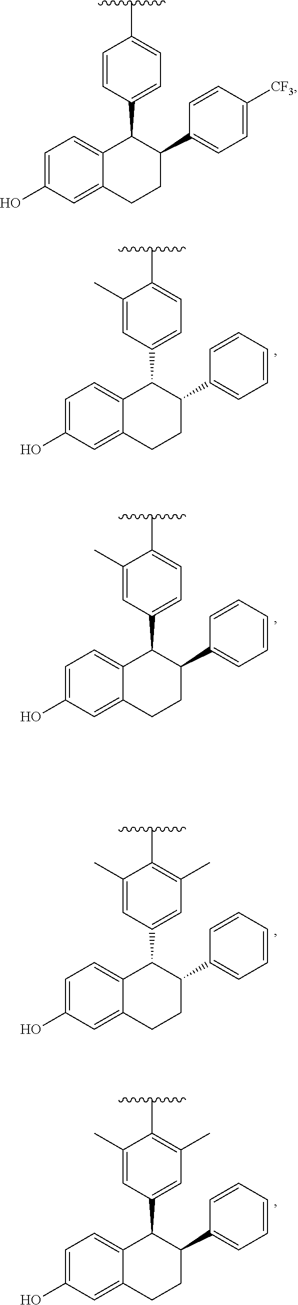 [SMILES: *c1ccc([C@@H]2c3ccc(O)cc3CC[C@@H]2c2ccc(C(F)(F)F)cc2)cc1 ; *c1ccc([C@H]2c3ccc(O)cc3CC[C@H]2c2ccccc2)cc1C ; *c1ccc([C@@H]2c3ccc(O)cc3CC[C@@H]2c2ccccc2)cc1C ; *c1c(C)cc([C@H]2c3ccc(O)cc3CC[C@H]2c2ccccc2)cc1C ; *c1c(C)cc([C@@H]2c3ccc(O)cc3CC[C@@H]2c2ccccc2)cc1C]

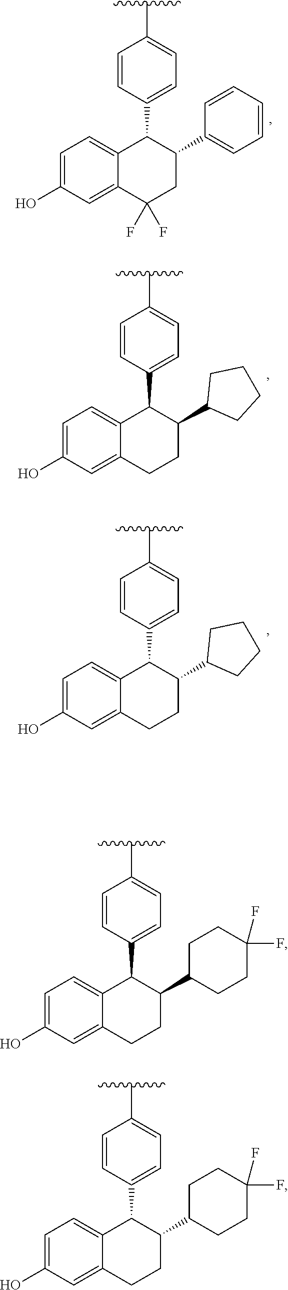

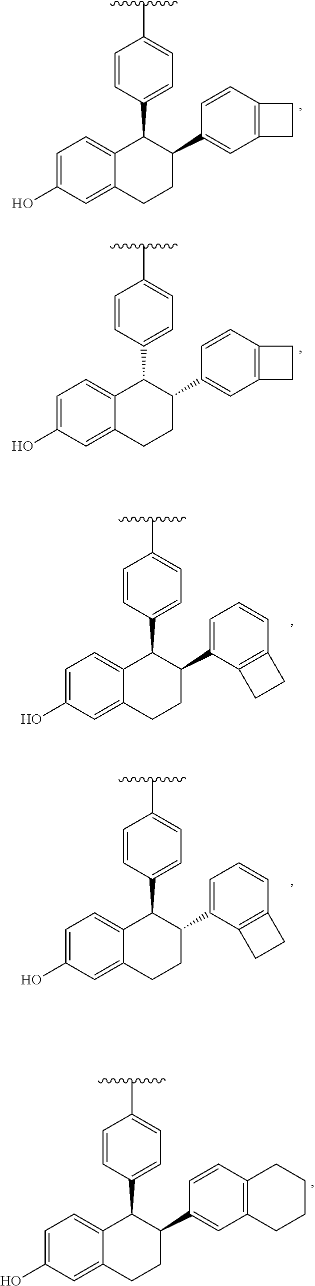

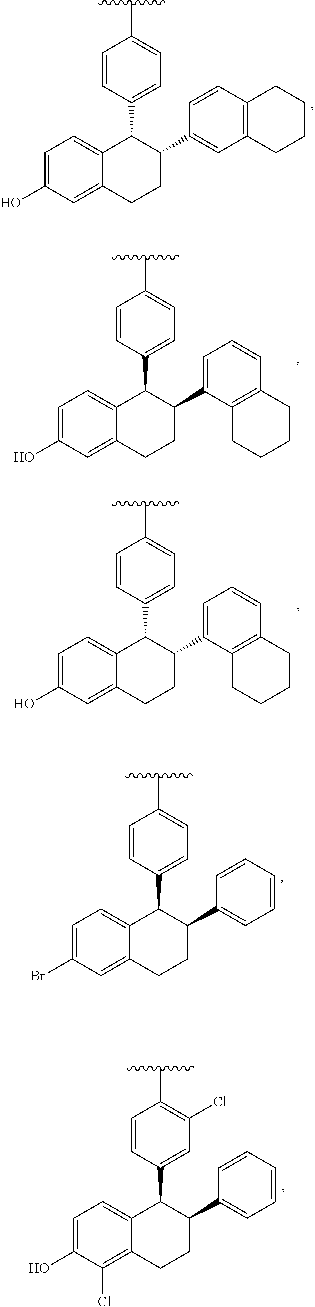

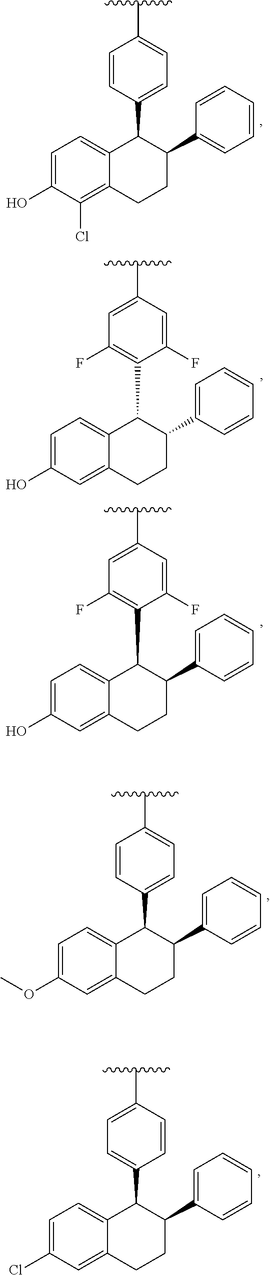

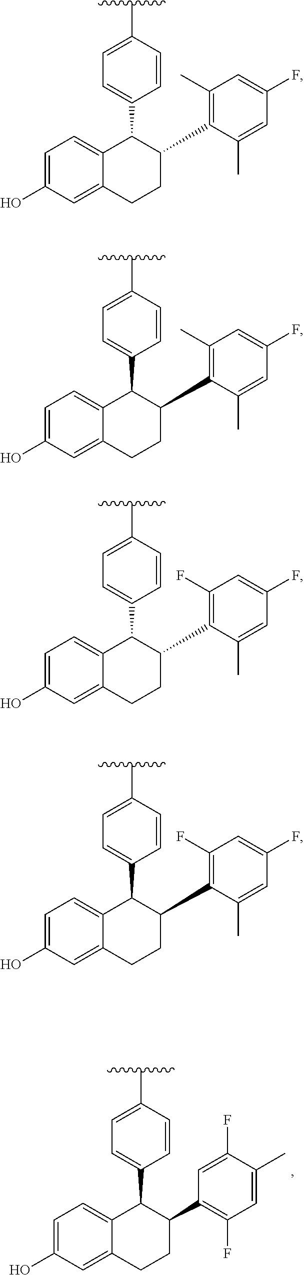

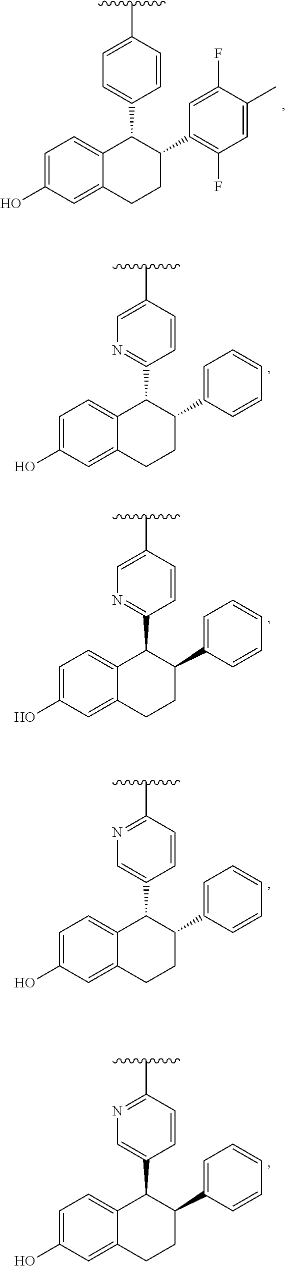 [SMILES: *c1ccc([C@H]2c3ccc(O)cc3CC[C@H]2c2cc(F)c(C)cc2F)cc1 ; *c1ccc([C@H]2c3ccc(O)cc3CC[C@H]2c2ccccc2)nc1 ; *c1ccc([C@@H]2c3ccc(O)cc3CC[C@@H]2c2ccccc2)nc1 ; *c1ccc([C@H]2c3ccc(O)cc3CC[C@H]2c2ccccc2)cn1 ; *c1ccc([C@@H]2c3ccc(O)cc3CC[C@@H]2c2ccccc2)cn1]

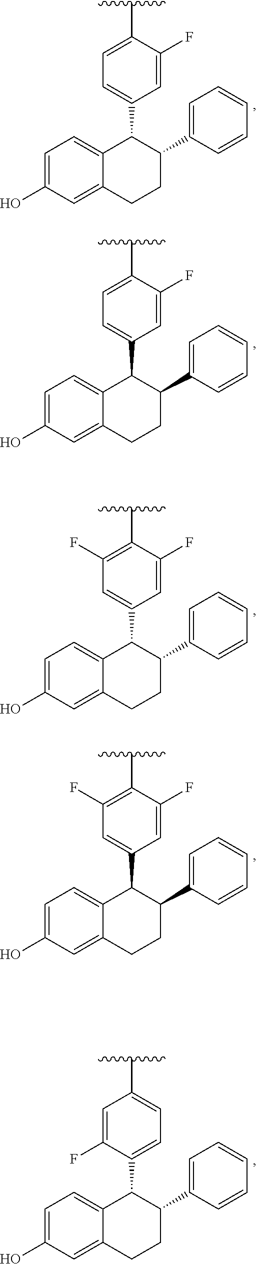

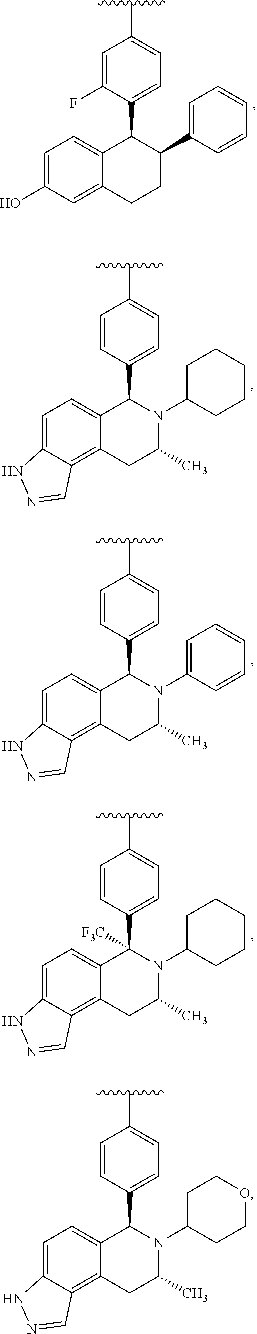 [SMILES: *c1ccc([C@@H]2c3ccc(O)cc3CC[C@@H]2c2ccccc2)c(F)c1 ; *c1ccc([C@@H]2c3ccc4[nH]ncc4c3C[C@@H](C)N2C2CCCCC2)cc1 ; *c1ccc([C@@H]2c3ccc4[nH]ncc4c3C[C@@H](C)N2c2ccccc2)cc1 ; *c1ccc([C@]2(C(F)(F)F)c3ccc4[nH]ncc4c3C[C@@H](C)N2C2CCCCC2)cc1 ; *c1ccc([C@@H]2c3ccc4[nH]ncc4c3C[C@@H](C)N2C2CCOCC2)cc1]

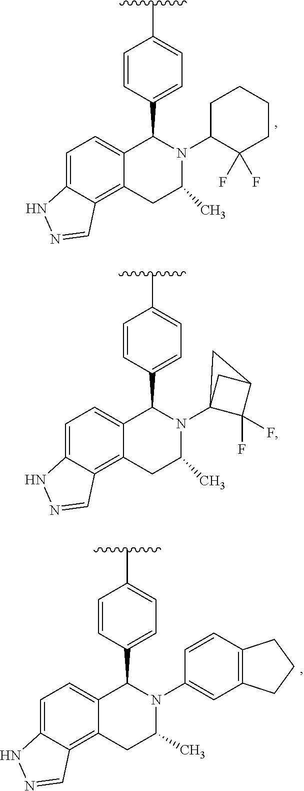 [SMILES: *c1ccc([C@@H]2c3ccc4[nH]ncc4c3C[C@@H](C)N2C2CCCCC2(F)F)cc1 ; *c1ccc([C@@H]2c3ccc4[nH]ncc4c3C[C@@H](C)N2C23CC(C2)C3(F)F)cc1 ; *c1ccc([C@@H]2c3ccc4[nH]ncc4c3C[C@@H](C)N2c2ccc3c(c2)CCC3)cc1]

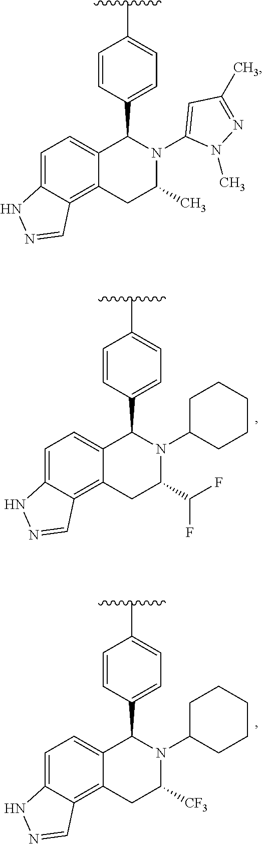 [SMILES: *c1ccc([C@@H]2c3ccc4[nH]ncc4c3C[C@@H](C)N2c2cc(C)nn2C)cc1 ; *c1ccc([C@@H]2c3ccc4[nH]ncc4c3C[C@@H](C(F)F)N2C2CCCCC2)cc1 ; *c1ccc([C@@H]2c3ccc4[nH]ncc4c3C[C@@H](C(F)(F)F)N2C2CCCCC2)cc1]

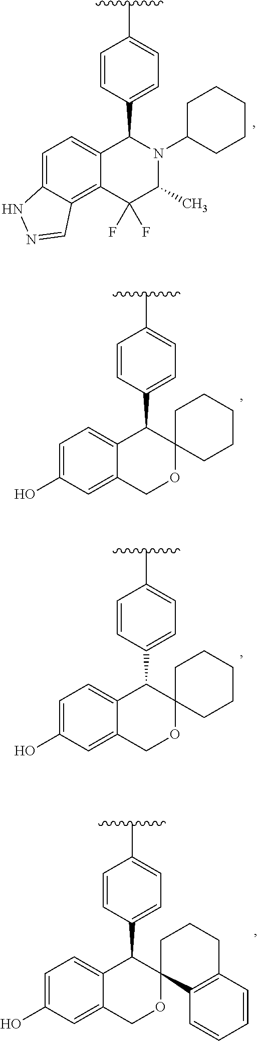 [SMILES: *c1ccc([C@@H]2c3ccc4[nH]ncc4c3C(F)(F)[C@@H](C)N2C2CCCCC2)cc1 ; *c1ccc([C@@H]2c3ccc(O)cc3COC23CCCCC3)cc1 ; *c1ccc([C@H]2c3ccc(O)cc3COC23CCCCC3)cc1 ; *c1ccc([C@@H]2c3ccc(O)cc3CO[C@@]23CCCc2ccccc23)cc1]

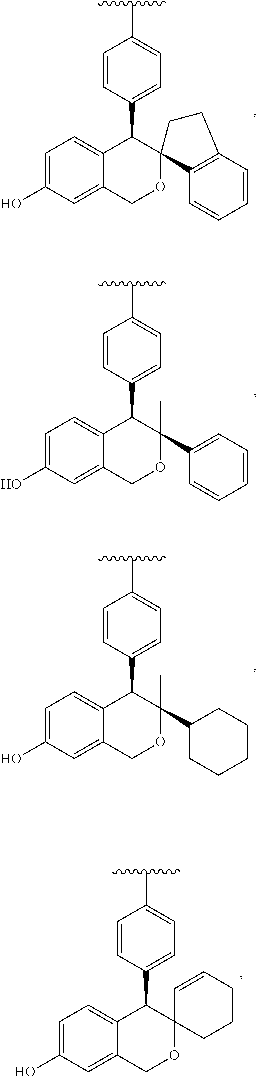 [SMILES: *c1ccc([C@@H]2c3ccc(O)cc3CO[C@@]23CCc2ccccc23)cc1 ; *c1ccc([C@@H]2c3ccc(O)cc3CO[C@]2(C)c2ccccc2)cc1 ; *c1ccc([C@@H]2c3ccc(O)cc3CO[C@]2(C)C2CCCCC2)cc1 ; *c1ccc([C@@H]2c3ccc(O)cc3COC23C=CCCC3)cc1]

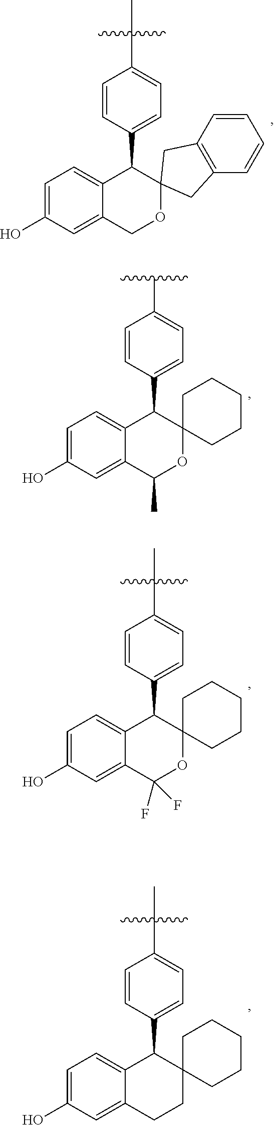 [SMILES: *c1ccc([C@@H]2c3ccc(O)cc3COC23Cc2ccccc2C3)cc1 ; *c1ccc([C@@H]2c3ccc(O)cc3[C@H](C)OC23CCCCC3)cc1 ; *c1ccc([C@@H]2c3ccc(O)cc3C(F)(F)OC23CCCCC3)cc1 ; *c1ccc([C@@H]2c3ccc(O)cc3CCC23CCCCC3)cc1]

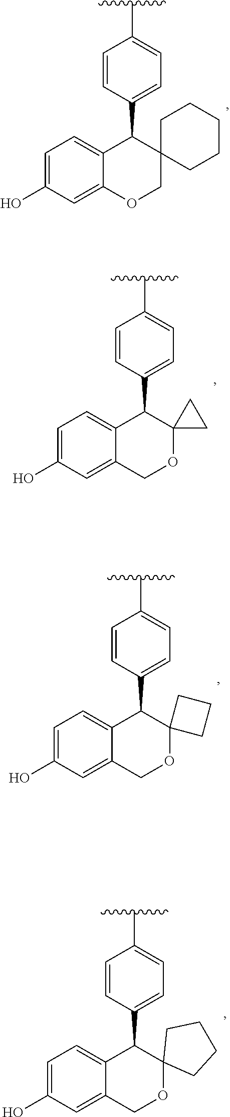 [SMILES: *c1ccc([C@@H]2c3ccc(O)cc3OCC23CCCCC3)cc1 ; *c1ccc([C@@H]2c3ccc(O)cc3COC23CC3)cc1 ; *c1ccc([C@@H]2c3ccc(O)cc3COC23CCC3)cc1 ; *c1ccc([C@@H]2c3ccc(O)cc3COC23CCCC3)cc1]

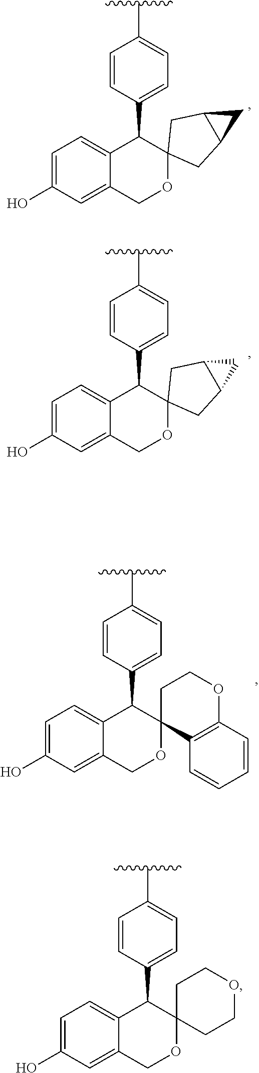 [SMILES: *c1ccc([C@@H]2c3ccc(O)cc3COC23C[C@H]2C[C@H]2C3)cc1 ; *c1ccc([C@@H]2c3ccc(O)cc3COC23C[C@H]2C[C@H]2C3)cc1 ; *c1ccc([C@@H]2c3ccc(O)cc3CO[C@@]23CCOc2ccccc23)cc1 ; *c1ccc([C@@H]2c3ccc(O)cc3COC23CCOCC3)cc1]

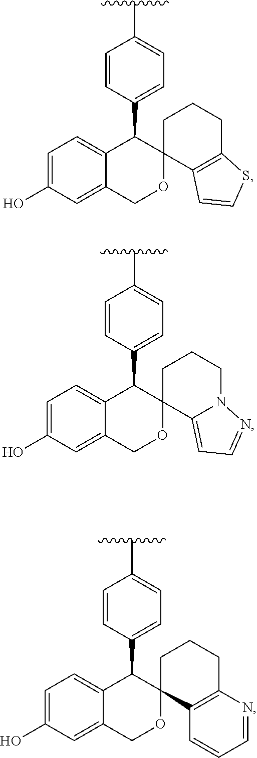 [SMILES: *c1ccc([C@@H]2c3ccc(O)cc3COC23CCCc2sccc23)cc1 ; *c1ccc([C@@H]2c3ccc(O)cc3COC23CCCn2nccc23)cc1 ; *c1ccc([C@@H]2c3ccc(O)cc3CO[C@@]23CCCc2ncccc23)cc1]

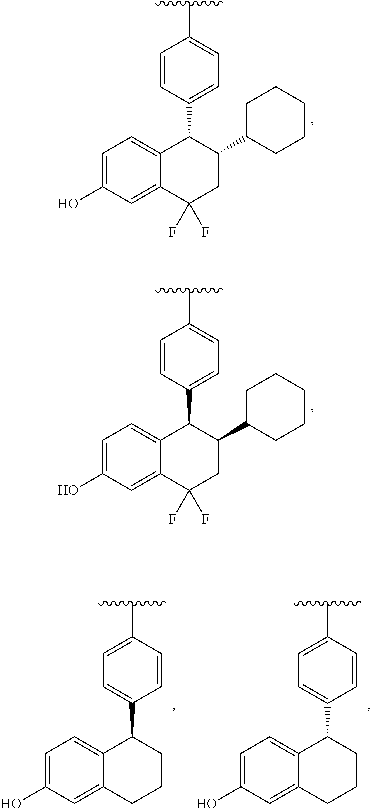 [SMILES: *c1ccc([C@H]2c3ccc(O)cc3C(F)(F)C[C@H]2C2CCCCC2)cc1 ; *c1ccc([C@@H]2c3ccc(O)cc3C(F)(F)C[C@@H]2C2CCCCC2)cc1 ; *c1ccc([C@H]2CCCc3cc(O)ccc32)cc1 ; *c1ccc([C@@H]2CCCc3cc(O)ccc32)cc1]

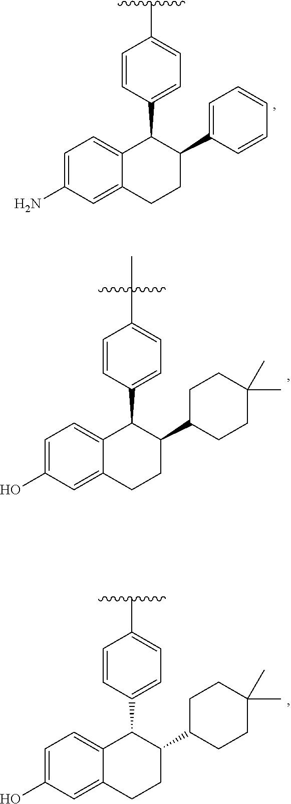 [SMILES: *c1ccc([C@@H]2c3ccc(N)cc3CC[C@@H]2c2ccccc2)cc1 ; *c1ccc([C@@H]2c3ccc(O)cc3CC[C@@H]2C2CCC(C)(C)CC2)cc1 ; *c1ccc([C@H]2c3ccc(O)cc3CC[C@H]2C2CCC(C)(C)CC2)cc1]

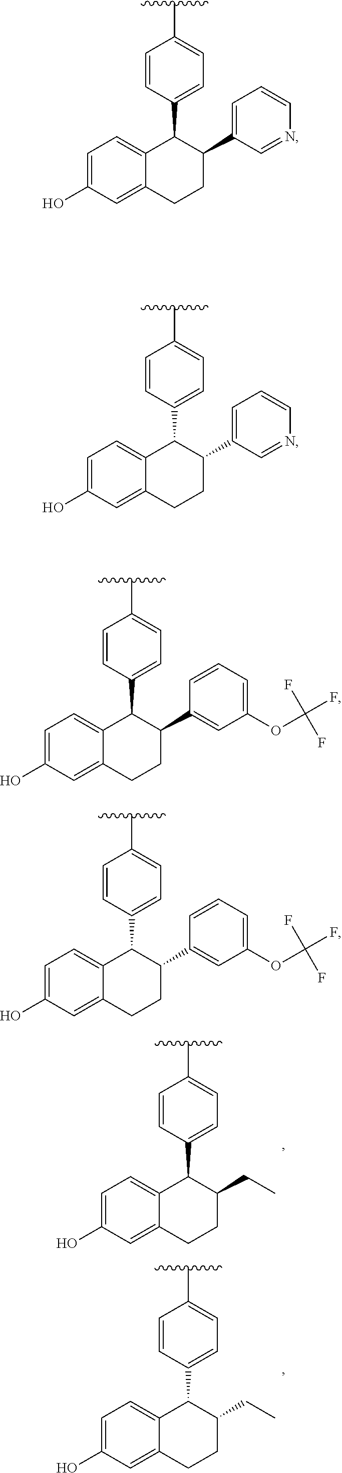

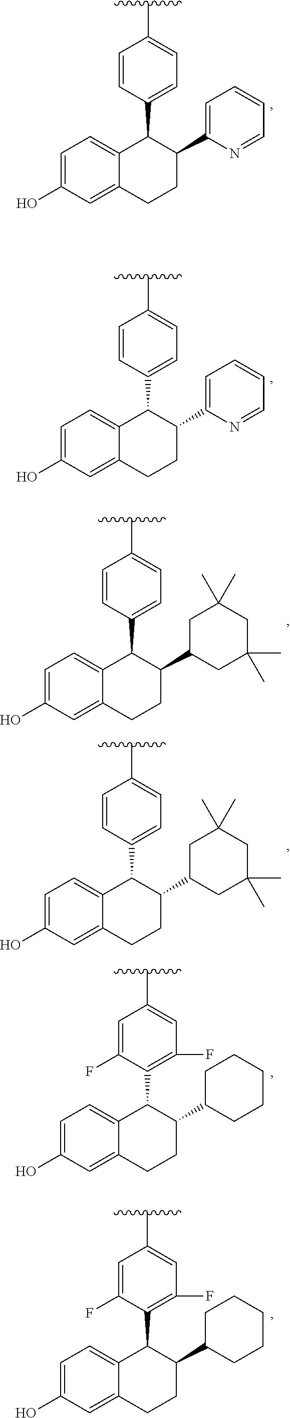

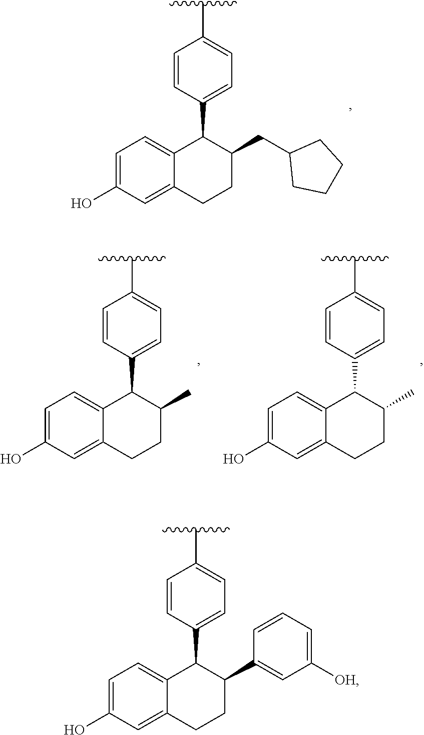 [SMILES: *c1ccc([C@@H]2c3ccc(O)cc3CC[C@@H]2CC2CCCC2)cc1 ; *c1ccc([C@@H]2c3ccc(O)cc3CC[C@@H]2C)cc1 ; *c1ccc([C@H]2c3ccc(O)cc3CC[C@H]2C)cc1 ; *c1ccc([C@@H]2c3ccc(O)cc3CC[C@@H]2c2cccc(O)c2)cc1]

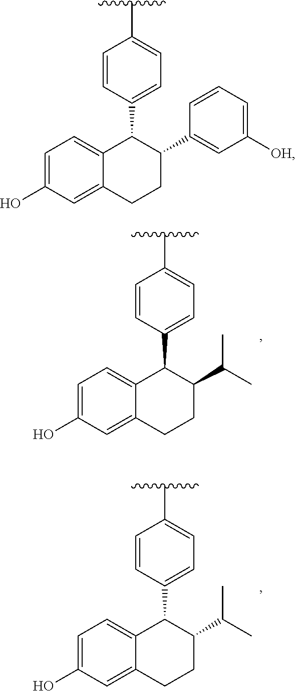

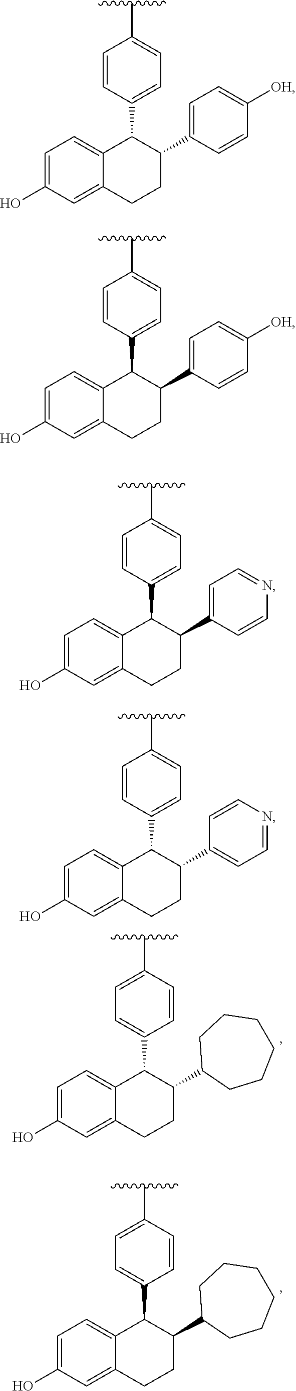

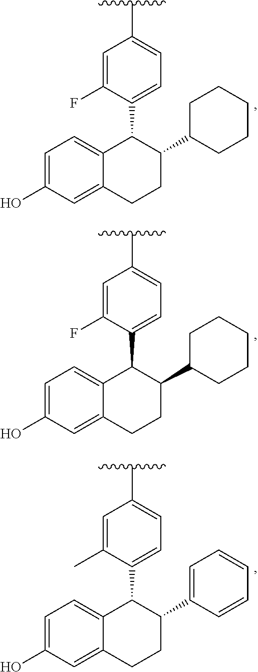

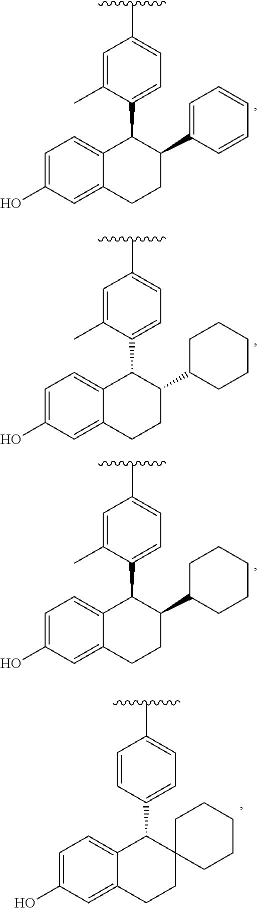

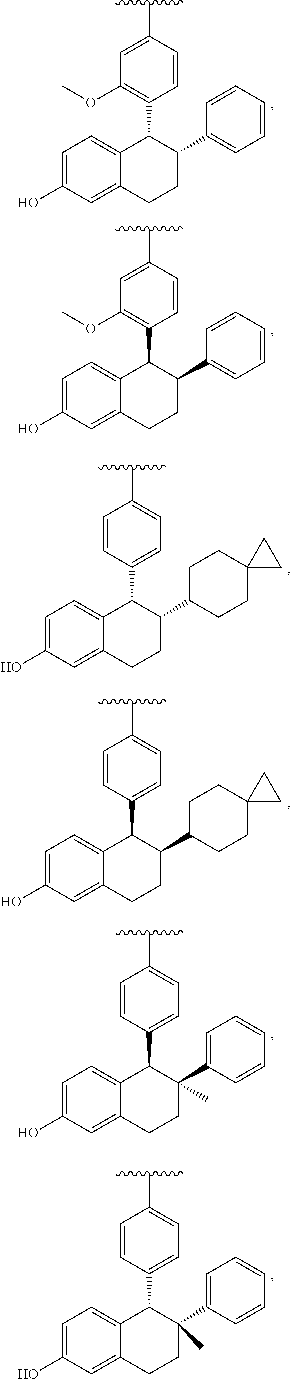

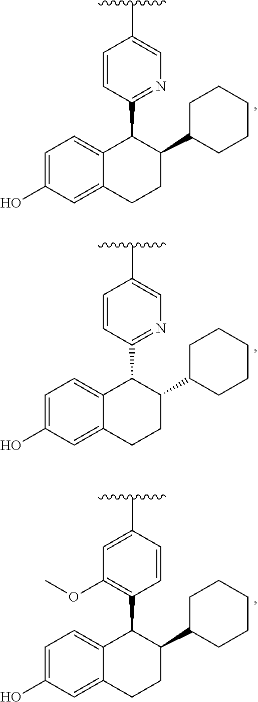

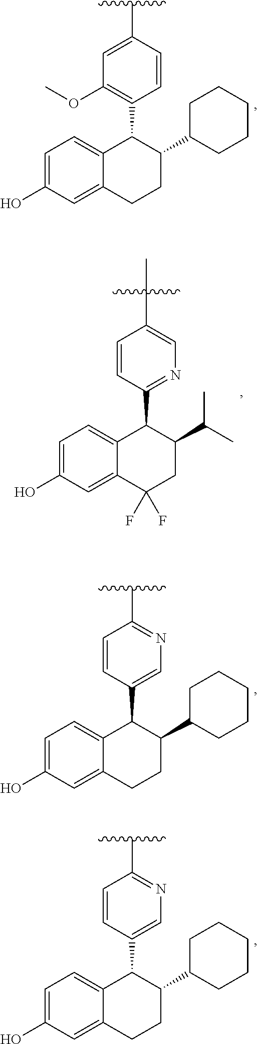 [SMILES: *c1ccc([C@H]2c3ccc(O)cc3CC[C@H]2C2CCCCC2)c(OC)c1 ; *c1ccc([C@@H]2c3ccc(O)cc3C(F)(F)C[C@@H]2C(C)C)nc1 ; *c1ccc([C@@H]2c3ccc(O)cc3CC[C@@H]2C2CCCCC2)cn1 ; *c1ccc([C@H]2c3ccc(O)cc3CC[C@H]2C2CCCCC2)cn1]

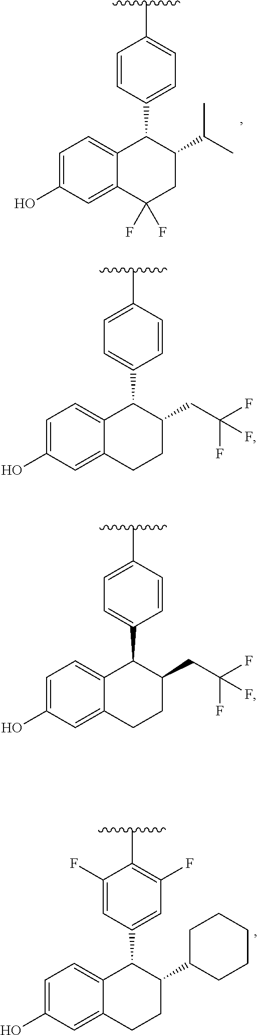

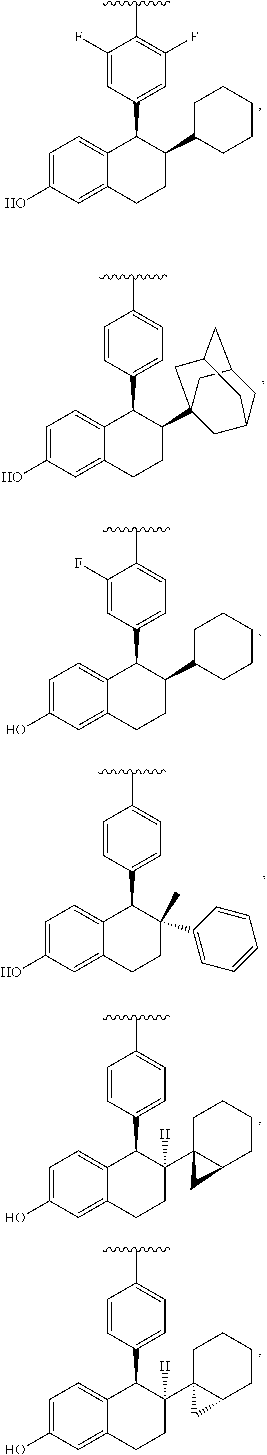

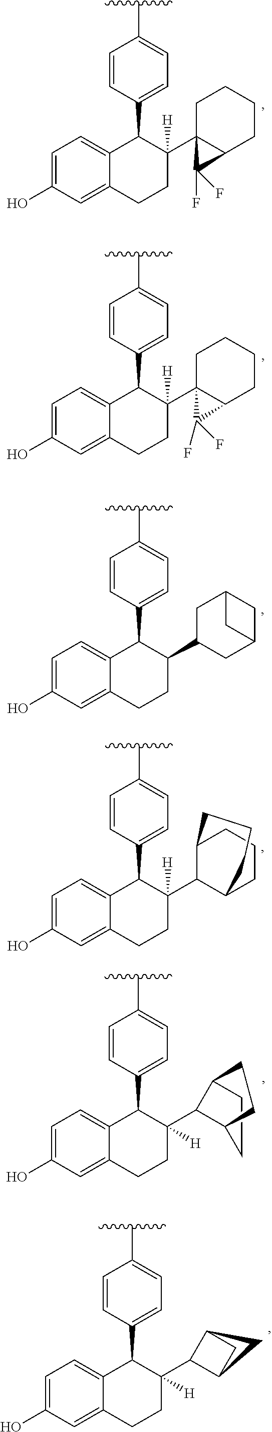

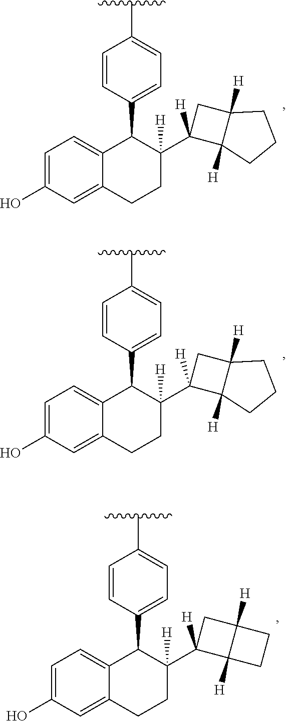 [SMILES: *c1ccc([C@@H]2c3ccc(O)cc3CC[C@@H]2[C@@H]2C[C@@H]3CCC[C@@H]32)cc1 ; *c1ccc([C@@H]2c3ccc(O)cc3CC[C@@H]2[C@H]2C[C@@H]3CCC[C@@H]32)cc1 ; *c1ccc([C@@H]2c3ccc(O)cc3CC[C@@H]2[C@@H]2C[C@@H]3CC[C@@H]32)cc1]

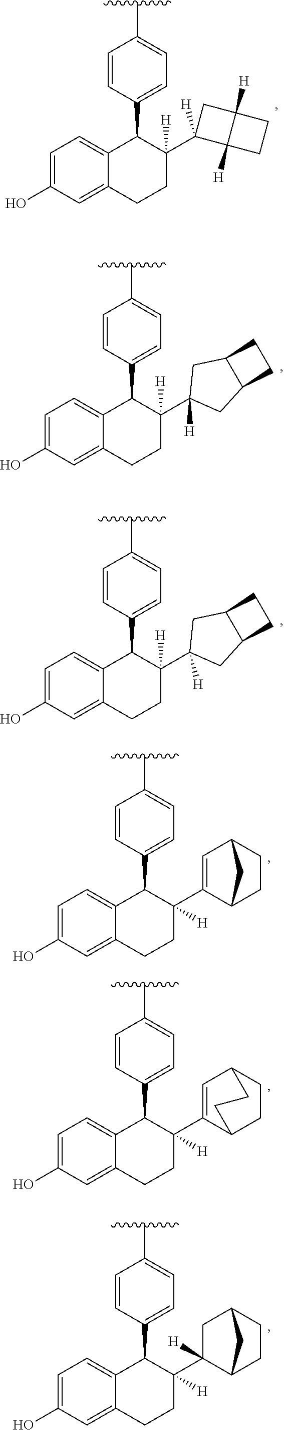

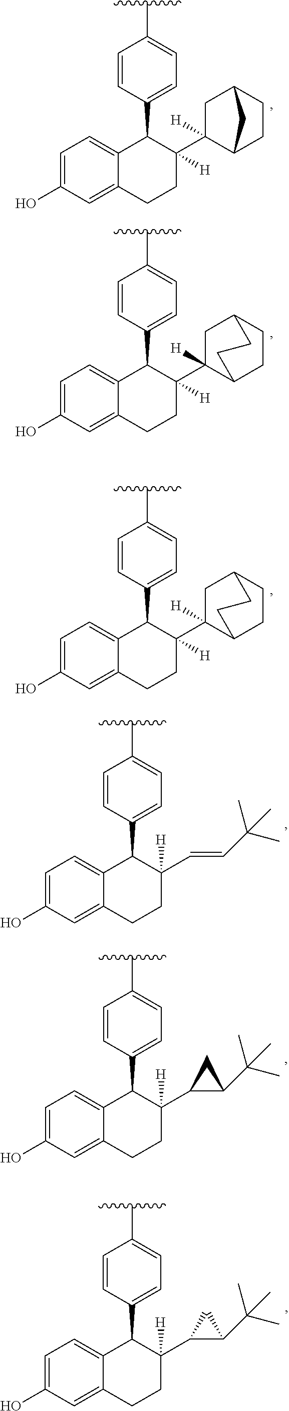

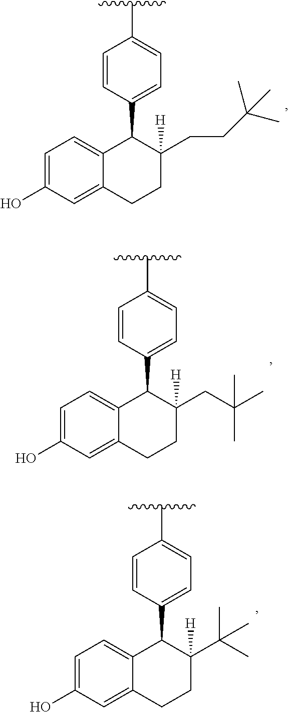 [SMILES: *c1ccc([C@@H]2c3ccc(O)cc3CC[C@@H]2CCC(C)(C)C)cc1 ; *c1ccc([C@@H]2c3ccc(O)cc3CC[C@@H]2CC(C)(C)C)cc1 ; *c1ccc([C@@H]2c3ccc(O)cc3CC[C@@H]2C(C)(C)C)cc1]

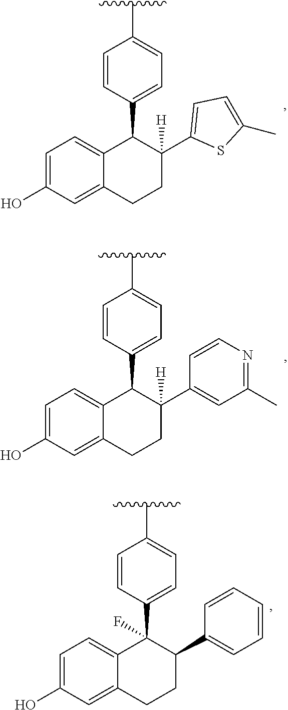 [SMILES: *c1ccc([C@@H]2c3ccc(O)cc3CC[C@@H]2c2ccc(C)s2)cc1 ; *c1ccc([C@@H]2c3ccc(O)cc3CC[C@@H]2c2ccnc(C)c2)cc1 ; *c1ccc([C@]2(F)c3ccc(O)cc3CC[C@@H]2c2ccccc2)cc1]

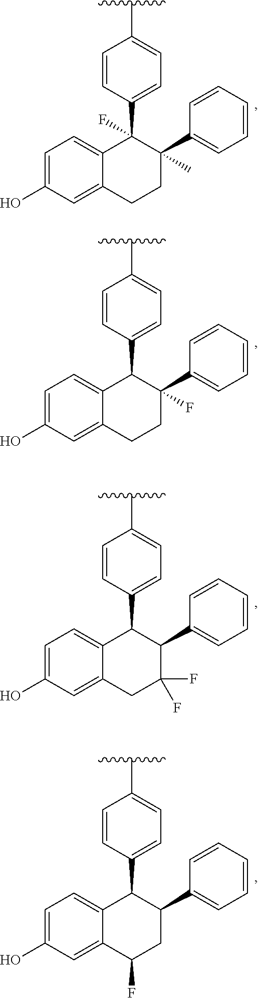

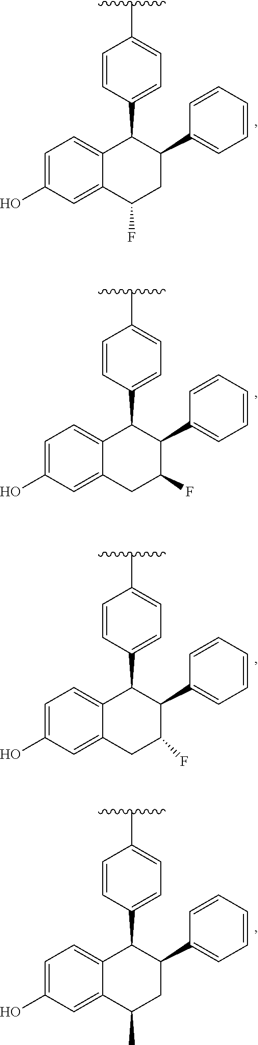 [SMILES: *c1ccc([C@@H]2c3ccc(O)cc3[C@@H](F)C[C@@H]2c2ccccc2)cc1 ; *c1ccc([C@@H]2c3ccc(O)cc3C[C@H](F)[C@@H]2c2ccccc2)cc1 ; *c1ccc([C@@H]2c3ccc(O)cc3C[C@@H](F)[C@@H]2c2ccccc2)cc1 ; *c1ccc([C@@H]2c3ccc(O)cc3[C@H](C)C[C@@H]2c2ccccc2)cc1]

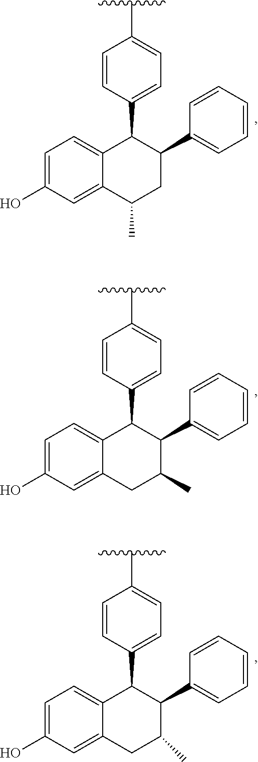 [SMILES: *c1ccc([C@@H]2c3ccc(O)cc3[C@@H](C)C[C@@H]2c2ccccc2)cc1 ; *c1ccc([C@@H]2c3ccc(O)cc3C[C@H](C)[C@@H]2c2ccccc2)cc1 ; *c1ccc([C@@H]2c3ccc(O)cc3C[C@@H](C)[C@@H]2c2ccccc2)cc1]

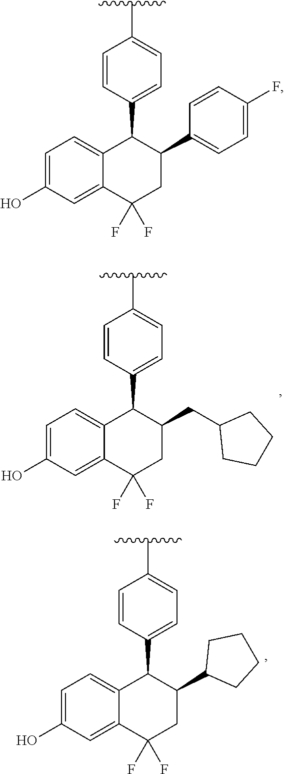

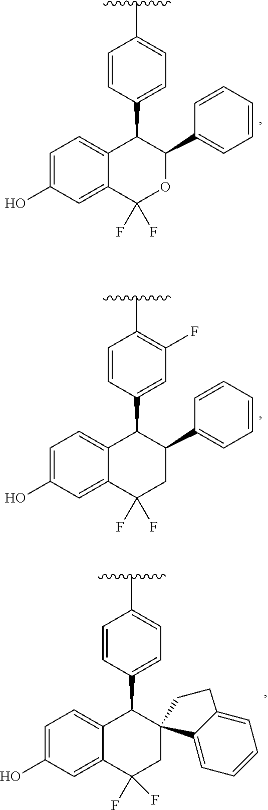 [SMILES: *c1ccc([C@@H]2c3ccc(O)cc3C(F)(F)O[C@@H]2c2ccccc2)cc1 ; *c1ccc([C@@H]2c3ccc(O)cc3C(F)(F)C[C@@H]2c2ccccc2)cc1F ; *c1ccc([C@@H]2c3ccc(O)cc3C(F)(F)C[C@@]23CCc2ccccc23)cc1]

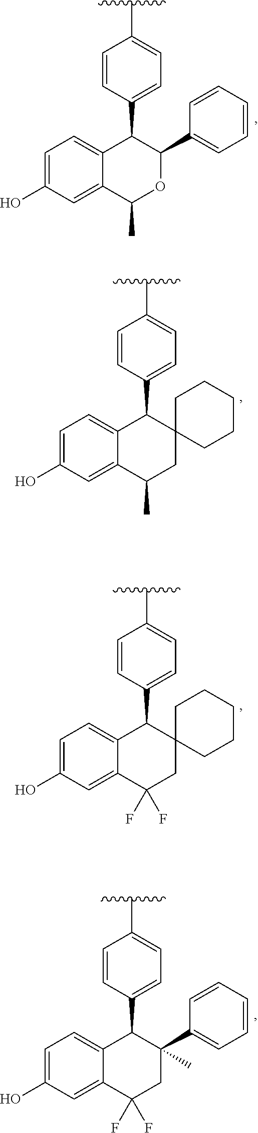 [SMILES: *c1ccc([C@@H]2c3ccc(O)cc3[C@H](C)O[C@@H]2c2ccccc2)cc1 ; *c1ccc([C@@H]2c3ccc(O)cc3[C@H](C)CC23CCCCC3)cc1 ; *c1ccc([C@@H]2c3ccc(O)cc3C(F)(F)CC23CCCCC3)cc1 ; *c1ccc([C@@H]2c3ccc(O)cc3C(F)(F)C[C@]2(C)c2ccccc2)cc1]

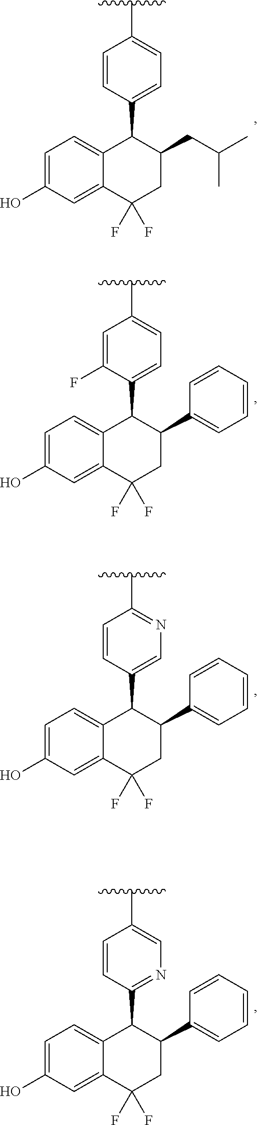 [SMILES: *c1ccc([C@@H]2c3ccc(O)cc3C(F)(F)C[C@@H]2CC(C)C)cc1 ; *c1ccc([C@@H]2c3ccc(O)cc3C(F)(F)C[C@@H]2c2ccccc2)c(F)c1 ; *c1ccc([C@@H]2c3ccc(O)cc3C(F)(F)C[C@@H]2c2ccccc2)cn1 ; *c1ccc([C@@H]2c3ccc(O)cc3C(F)(F)C[C@@H]2c2ccccc2)nc1]

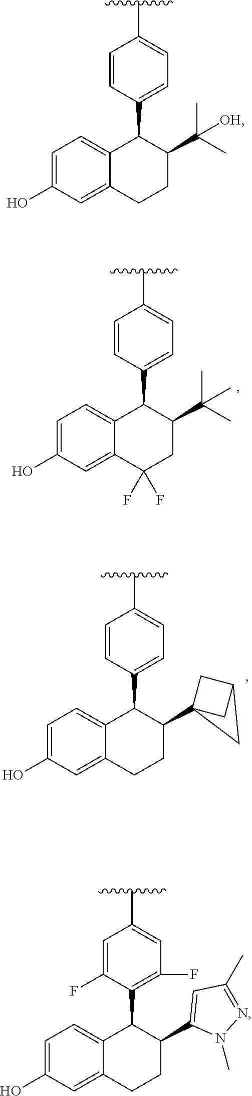 [SMILES: *c1ccc([C@@H]2c3ccc(O)cc3CC[C@@H]2C(C)(C)O)cc1 ; *c1ccc([C@@H]2c3ccc(O)cc3C(F)(F)C[C@@H]2C(C)(C)C)cc1 ; *c1ccc([C@@H]2c3ccc(O)cc3CC[C@@H]2C23CC(C2)C3)cc1 ; *c1cc(F)c([C@@H]2c3ccc(O)cc3CC[C@@H]2c2cc(C)nn2C)c(F)c1]

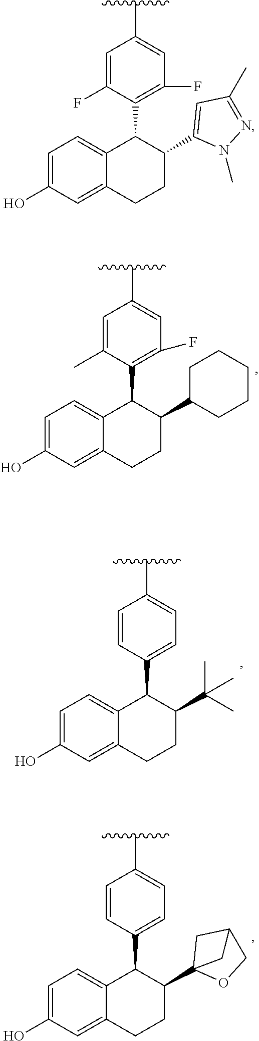 [SMILES: *c1cc(F)c([C@H]2c3ccc(O)cc3CC[C@H]2c2cc(C)nn2C)c(F)c1 ; *c1cc(C)c([C@@H]2c3ccc(O)cc3CC[C@@H]2C2CCCCC2)c(F)c1 ; *c1ccc([C@@H]2c3ccc(O)cc3CC[C@@H]2C(C)(C)C)cc1 ; *c1ccc([C@@H]2c3ccc(O)cc3CC[C@@H]2C23CC(CO2)C3)cc1]

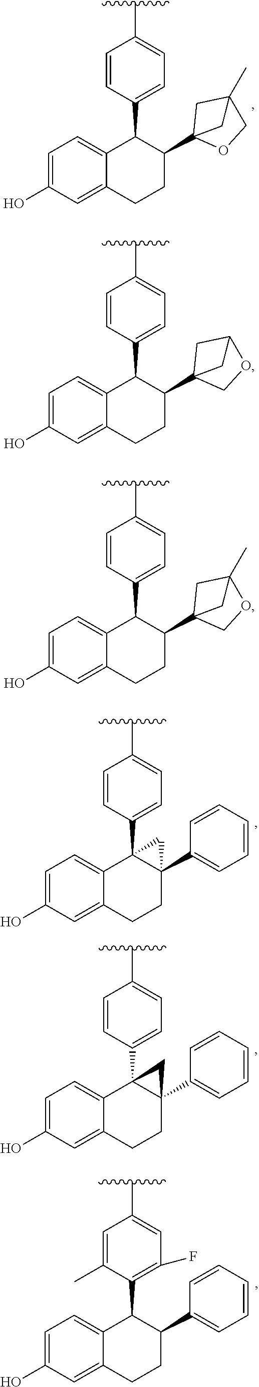

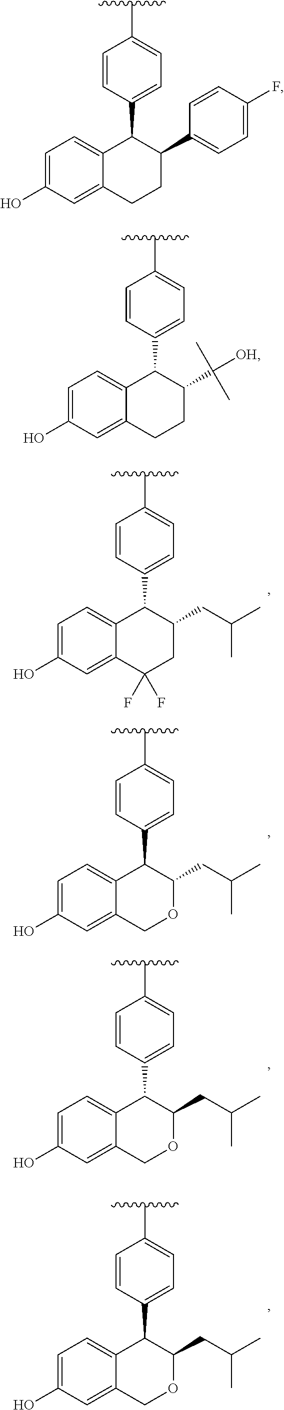

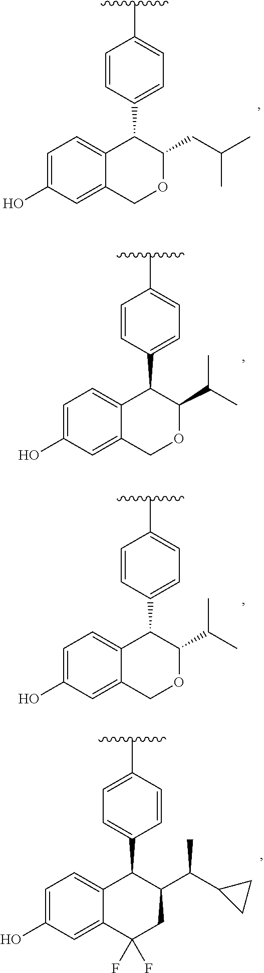

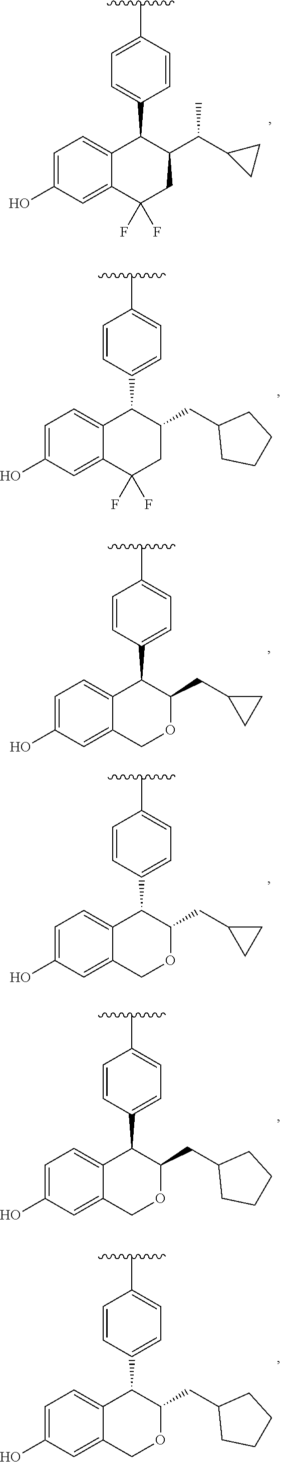

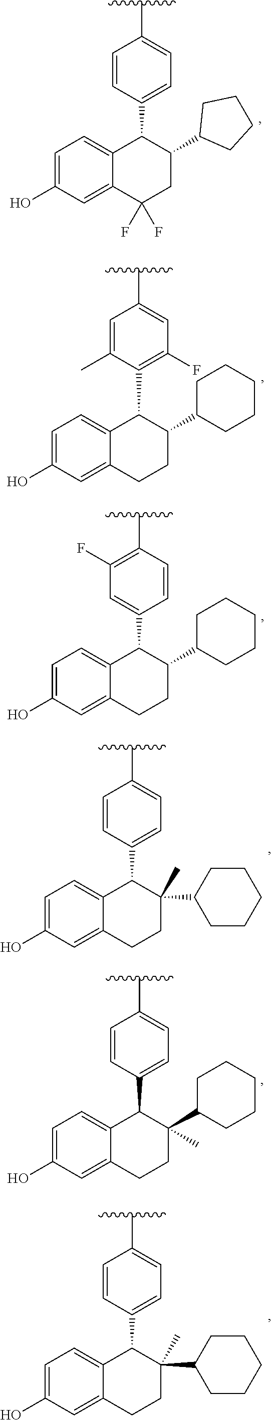

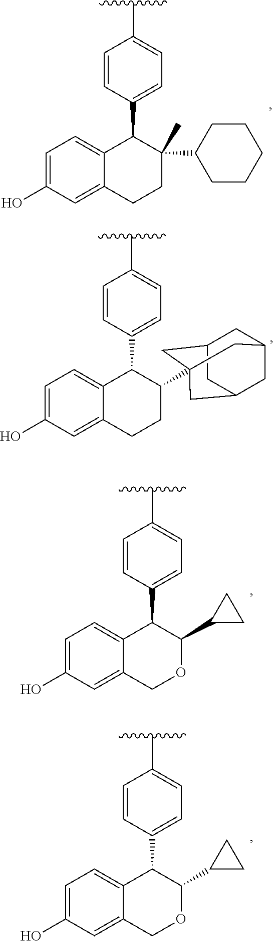

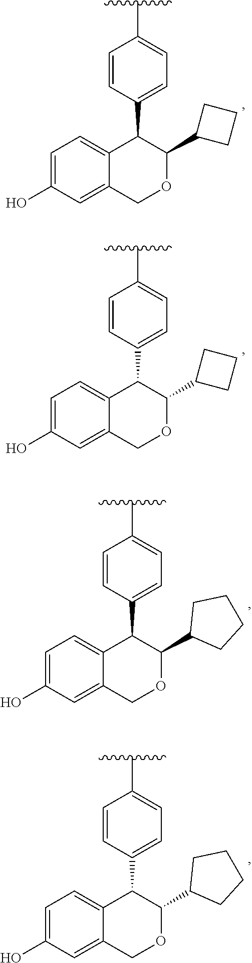

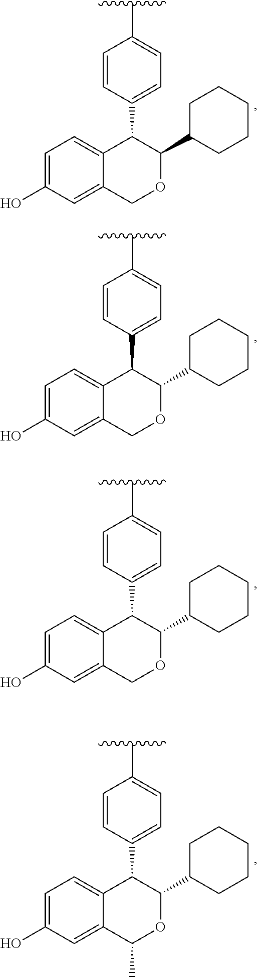

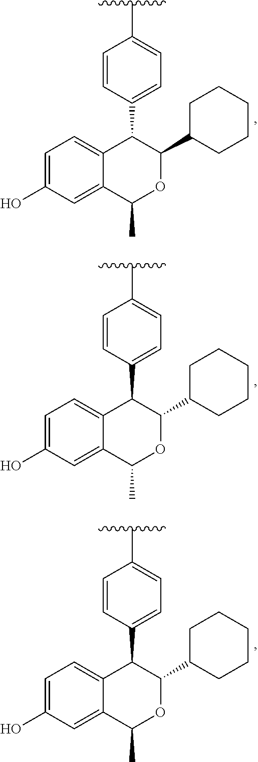

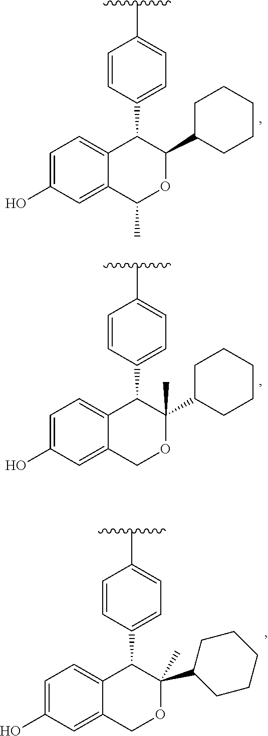

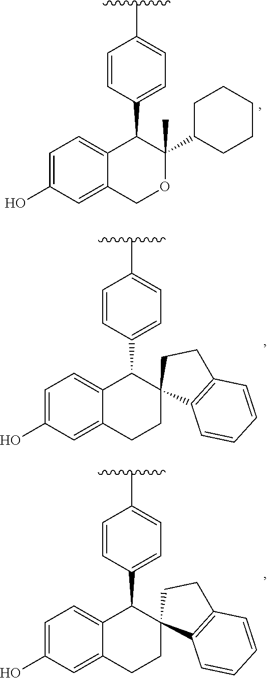

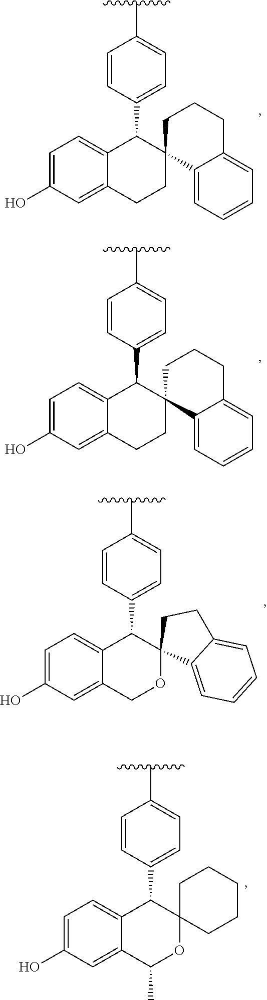

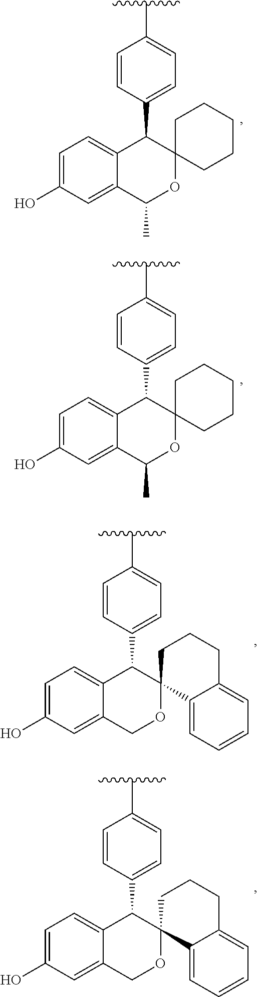

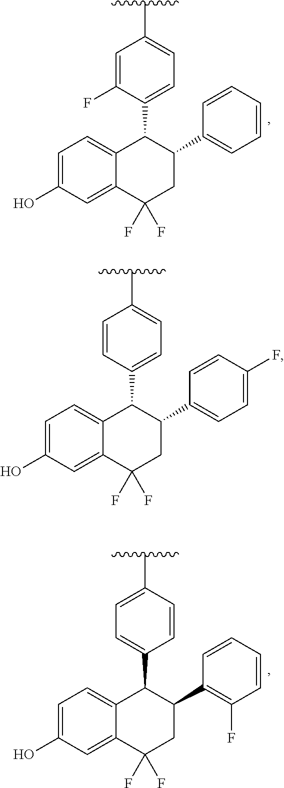

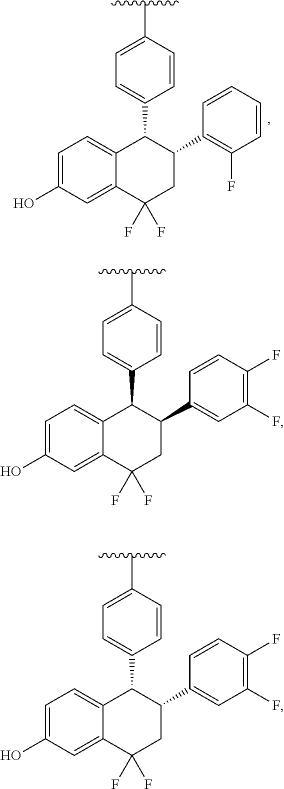

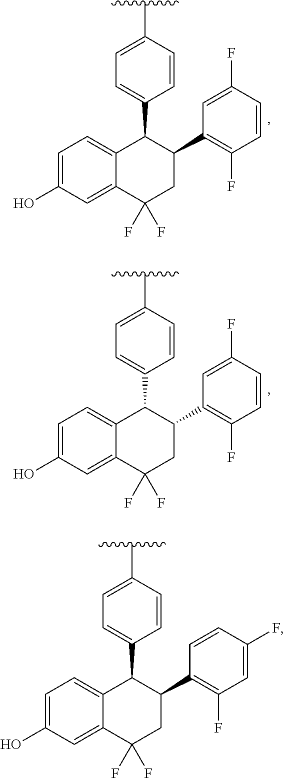

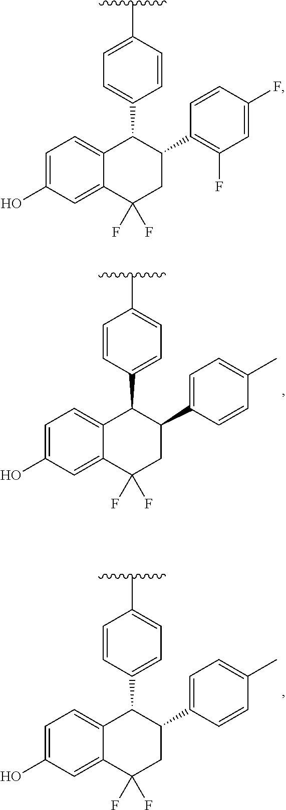

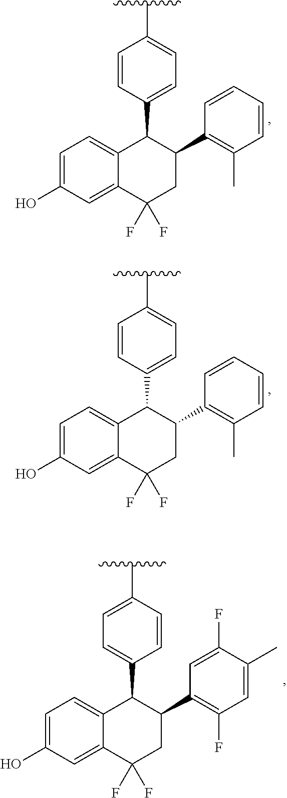

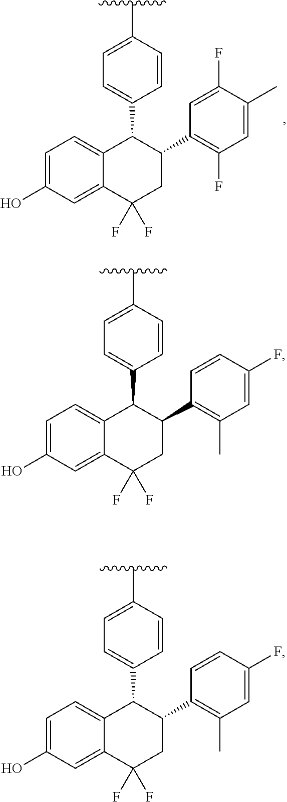

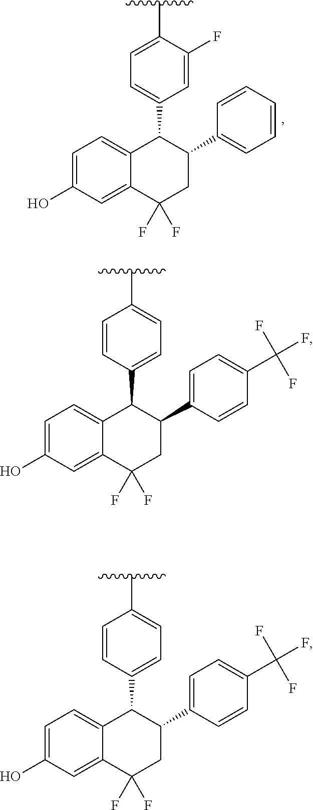

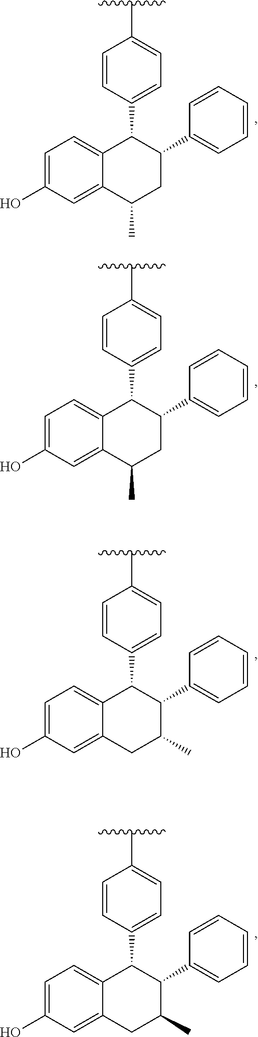

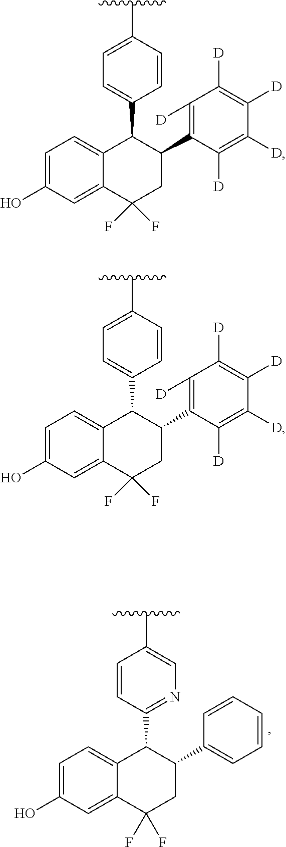

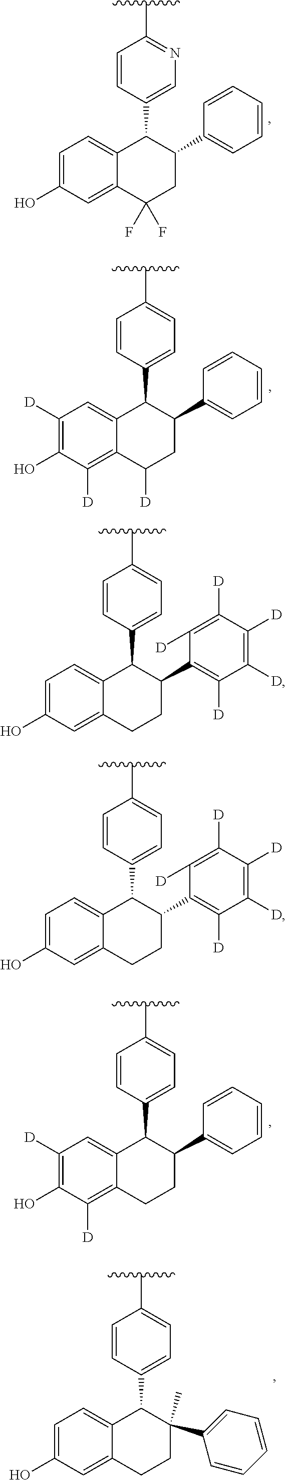

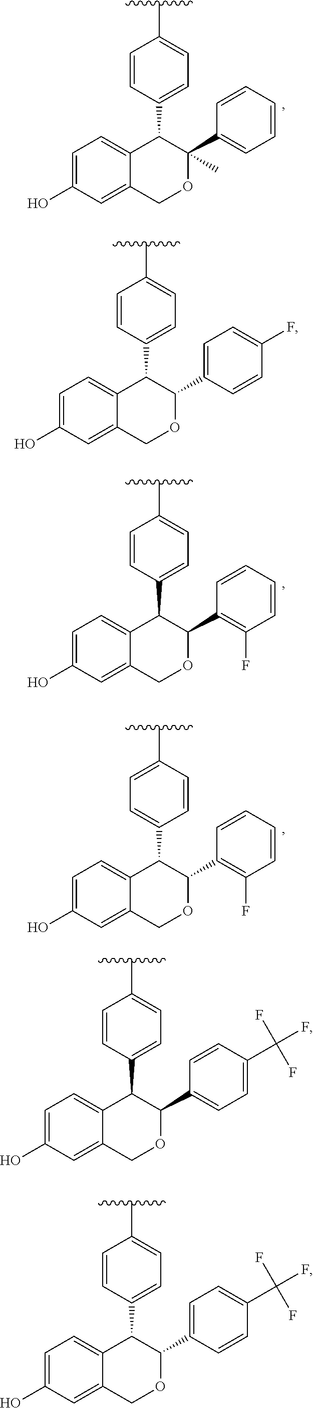

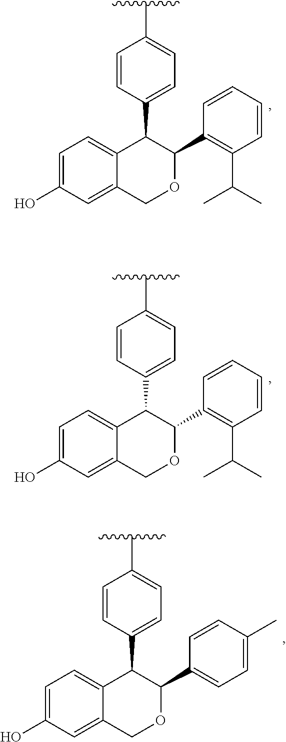 [SMILES: *c1ccc([C@@H]2c3ccc(O)cc3CO[C@@H]2c2ccccc2C(C)C)cc1 ; *c1ccc([C@H]2c3ccc(O)cc3CO[C@H]2c2ccccc2C(C)C)cc1 ; *c1ccc([C@@H]2c3ccc(O)cc3CO[C@@H]2c2ccc(C)cc2)cc1]

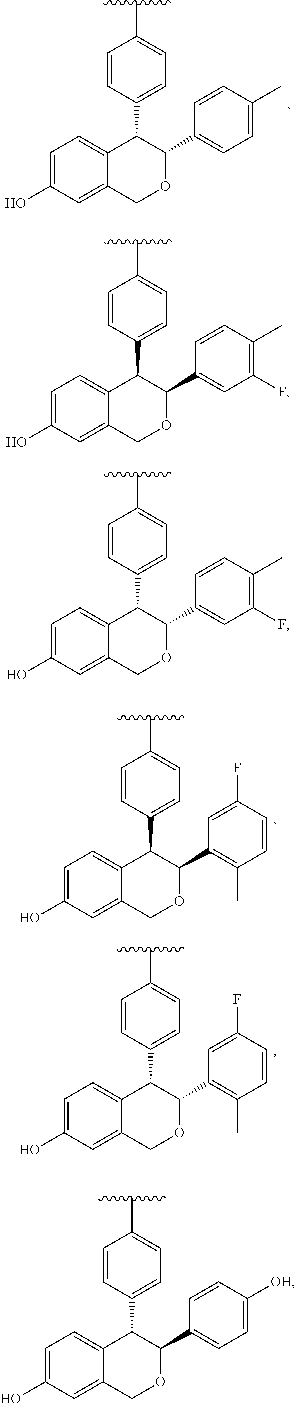

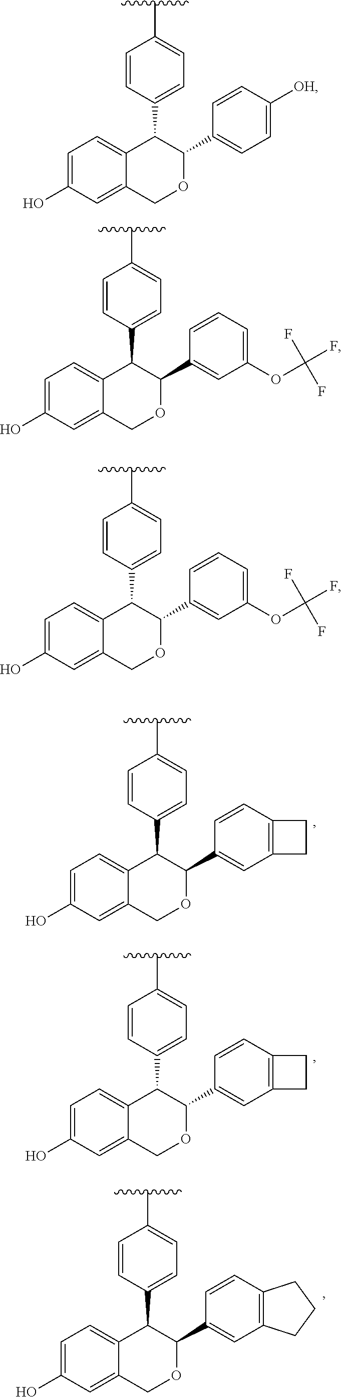

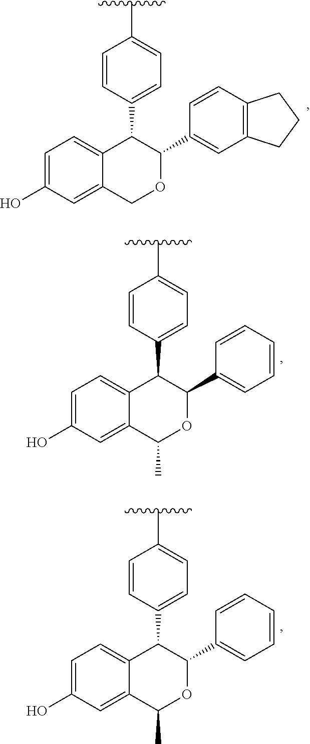

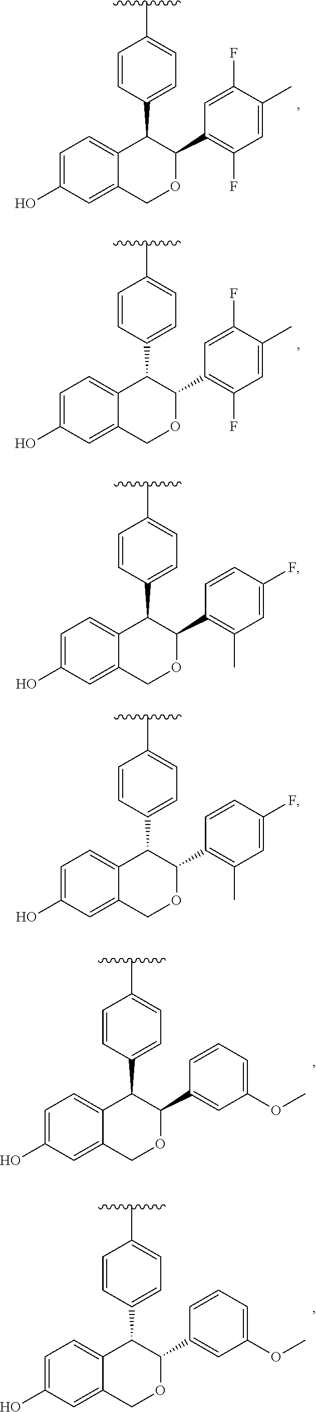

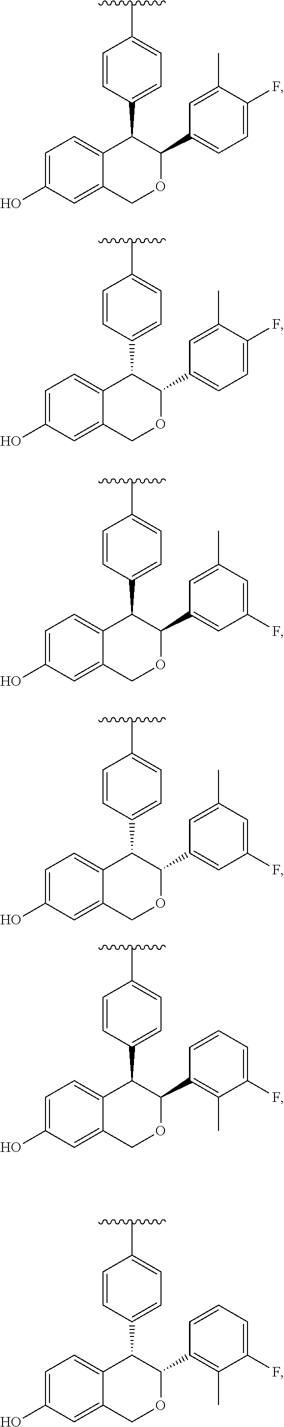

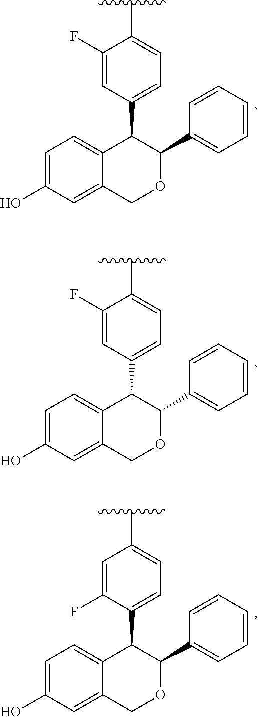 [SMILES: *c1ccc([C@@H]2c3ccc(O)cc3CO[C@@H]2c2ccccc2)cc1F ; *c1ccc([C@H]2c3ccc(O)cc3CO[C@H]2c2ccccc2)cc1F ; *c1ccc([C@@H]2c3ccc(O)cc3CO[C@@H]2c2ccccc2)c(F)c1]

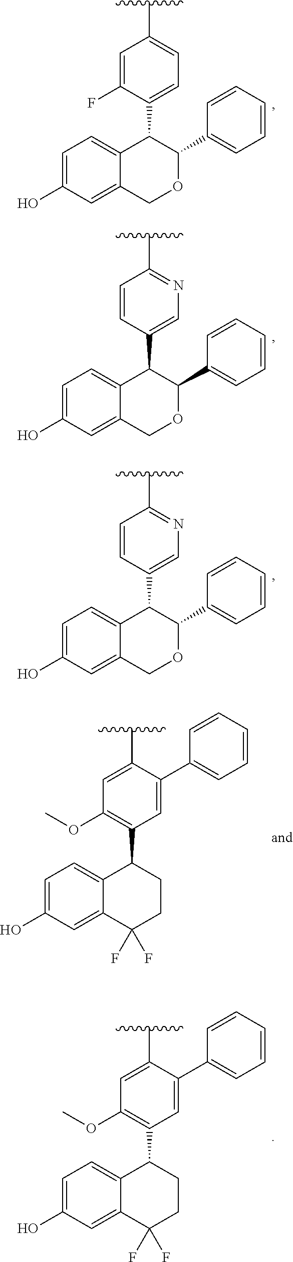

In some embodiments, ERBM is

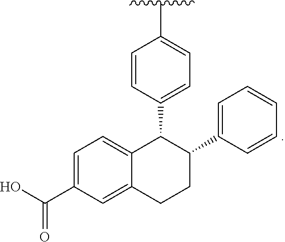 [SMILES: *c1ccc([C@H]2c3ccc(C(=O)O)cc3CC[C@H]2c2ccccc2)cc1]

In some embodiments, ERBM is

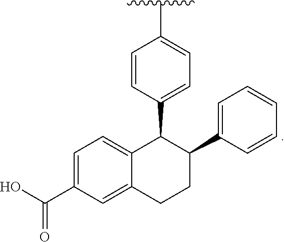 [SMILES: *c1ccc([C@@H]2c3ccc(C(=O)O)cc3CC[C@@H]2c2ccccc2)cc1]

In some embodiments, ERBM is

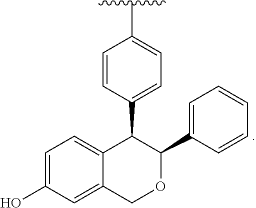 [SMILES: *c1ccc([C@@H]2c3ccc(O)cc3CO[C@@H]2c2ccccc2)cc1]

In some embodiments, ERBM is

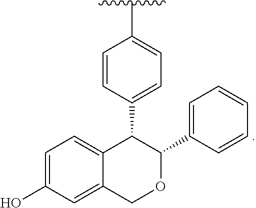 [SMILES: *c1ccc([C@H]2c3ccc(O)cc3CO[C@H]2c2ccccc2)cc1]

In some embodiments, ERBM is

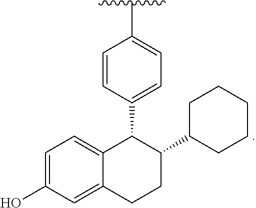 [SMILES: *c1ccc([C@H]2c3ccc(O)cc3CC[C@H]2C2CCCCC2)cc1]

In some embodiments, ERBM is

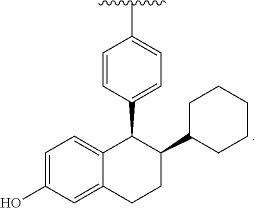 [SMILES: *c1ccc([C@@H]2c3ccc(O)cc3CC[C@@H]2C2CCCCC2)cc1]

In some embodiments, ERBM is

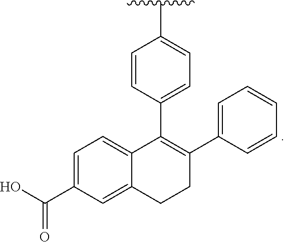 [SMILES: *c1ccc(C2=C(c3ccccc3)CCc3cc(C(=O)O)ccc32)cc1]

In some embodiments, ERBM is

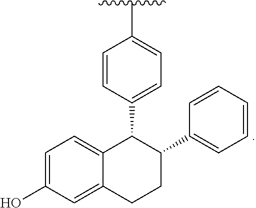 [SMILES: *c1ccc([C@H]2c3ccc(O)cc3CC[C@H]2c2ccccc2)cc1]

In some embodiments, ERBM is

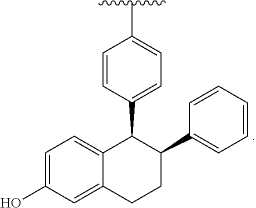 [SMILES: *c1ccc([C@@H]2c3ccc(O)cc3CC[C@@H]2c2ccccc2)cc1]

In some embodiments, ERBM is

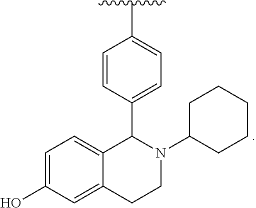 [SMILES: *c1ccc(C2c3ccc(O)cc3CCN2C2CCCCC2)cc1]

In some embodiments, ERBM is

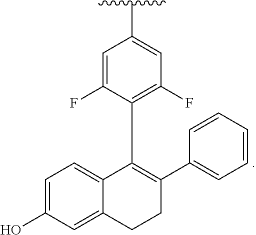 [SMILES: *c1cc(F)c(C2=C(c3ccccc3)CCc3cc(O)ccc32)c(F)c1]

In some embodiments, ERBM is

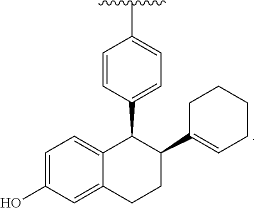 [SMILES: *c1ccc([C@@H]2c3ccc(O)cc3CC[C@@H]2C2=CCCCC2)cc1]

In some embodiments, ERBM is

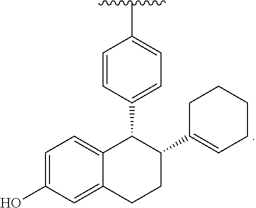 [SMILES: *c1ccc([C@H]2c3ccc(O)cc3CC[C@H]2C2=CCCCC2)cc1]

In some embodiments, ERBM is

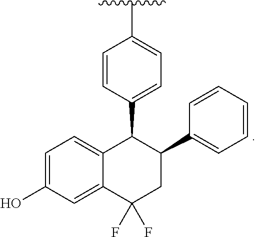 [SMILES: *c1ccc([C@@H]2c3ccc(O)cc3C(F)(F)C[C@@H]2c2ccccc2)cc1]

In some embodiments, ERBM is

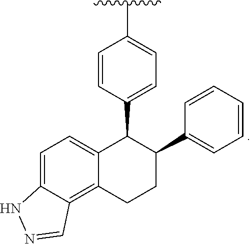 [SMILES: *c1ccc([C@@H]2c3ccc4[nH]ncc4c3CC[C@@H]2c2ccccc2)cc1]

In some embodiments, ERBM is

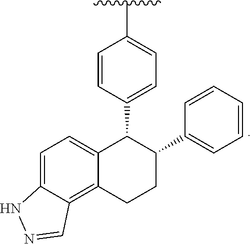 [SMILES: *c1ccc([C@H]2c3ccc4[nH]ncc4c3CC[C@H]2c2ccccc2)cc1]

In some embodiments, ERBM is

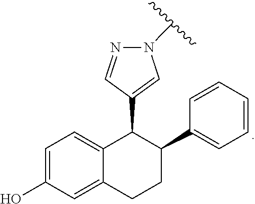 [SMILES: *n1cc([C@@H]2c3ccc(O)cc3CC[C@@H]2c2ccccc2)cn1]

In some embodiments, ERBM is

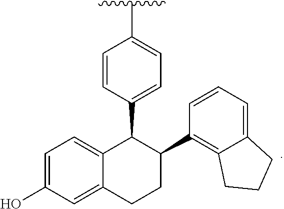 [SMILES: *c1ccc([C@@H]2c3ccc(O)cc3CC[C@@H]2c2cccc3c2CCC3)cc1]

In some embodiments, ERBM is

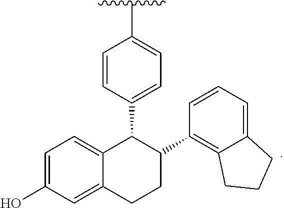

In some embodiments, ERBM is

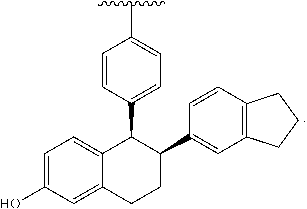 [SMILES: *c1ccc([C@@H]2c3ccc(O)cc3CC[C@@H]2c2ccc3c(c2)CCC3)cc1]

In some embodiments, ERBM is

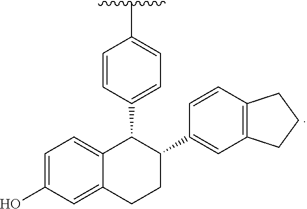 [SMILES: *c1ccc([C@H]2c3ccc(O)cc3CC[C@H]2c2ccc3c(c2)CCC3)cc1]

In some embodiments, ERBM is

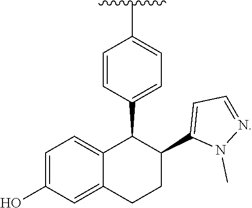 [SMILES: *c1ccc([C@@H]2c3ccc(O)cc3CC[C@@H]2c2ccnn2C)cc1]

In some embodiments, ERBM is

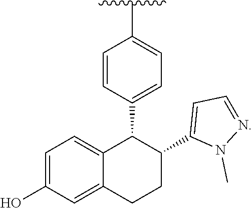 [SMILES: *c1ccc([C@H]2c3ccc(O)cc3CC[C@H]2c2ccnn2C)cc1]

In some embodiments, ERBM is

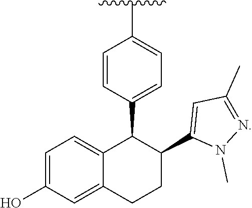 [SMILES: *c1ccc([C@@H]2c3ccc(O)cc3CC[C@@H]2c2cc(C)nn2C)cc1]

In some embodiments, ERBM is

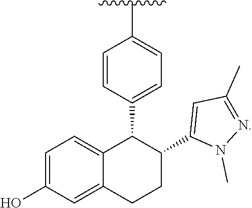 [SMILES: *c1ccc([C@H]2c3ccc(O)cc3CC[C@H]2c2cc(C)nn2C)cc1]

In some embodiments, ERBM is

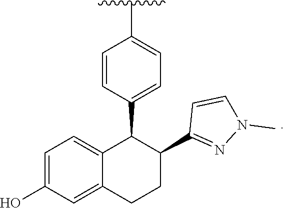 [SMILES: *c1ccc([C@@H]2c3ccc(O)cc3CC[C@@H]2c2ccn(C)n2)cc1]

In some embodiments, ERBM is

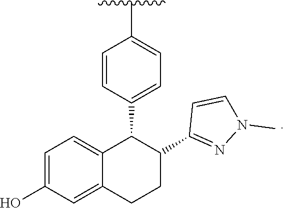

In some embodiments, ERBM is

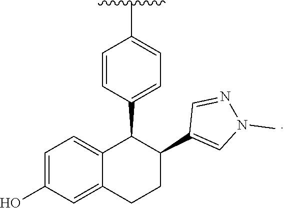 [SMILES: *c1ccc([C@@H]2c3ccc(O)cc3CC[C@@H]2c2cnn(C)c2)cc1]

In some embodiments, ERBM is

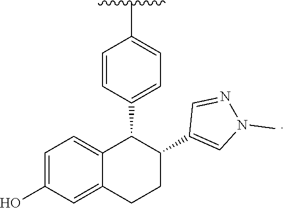 [SMILES: *c1ccc([C@H]2c3ccc(O)cc3CC[C@H]2c2cnn(C)c2)cc1]

In some embodiments, ERBM is

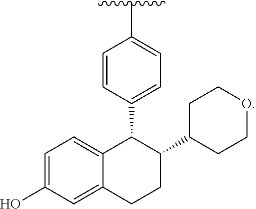 [SMILES: *c1ccc([C@H]2c3ccc(O)cc3CC[C@H]2C2CCOCC2)cc1]

In some embodiments, ERBM is

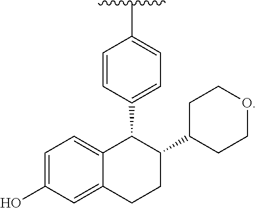 [SMILES: *c1ccc([C@H]2c3ccc(O)cc3CC[C@H]2C2CCOCC2)cc1]

In some embodiments, ERBM is

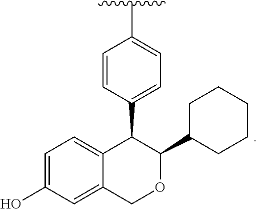 [SMILES: *c1ccc([C@@H]2c3ccc(O)cc3CO[C@@H]2C2CCCCC2)cc1]

In some embodiments, ERBM is

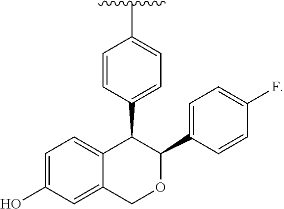 [SMILES: *c1ccc([C@@H]2c3ccc(O)cc3CO[C@@H]2c2ccc(F)cc2)cc1]

In some embodiments, ERBM is

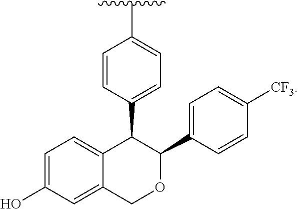 [SMILES: *c1ccc([C@@H]2c3ccc(O)cc3CO[C@@H]2c2ccc(C(F)(F)F)cc2)cc1]

In some embodiments, ERBM is

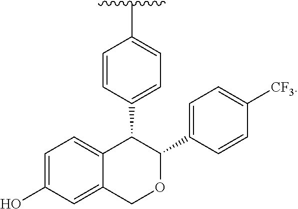 [SMILES: *c1ccc([C@H]2c3ccc(O)cc3CO[C@H]2c2ccc(C(F)(F)F)cc2)cc1]

In some embodiments, ERBM is

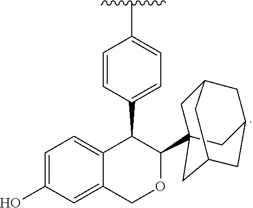 [SMILES: *c1ccc([C@@H]2c3ccc(O)cc3CO[C@@H]2C23CC4CC(CC(C4)C2)C3)cc1]

In some embodiments, ERBM is

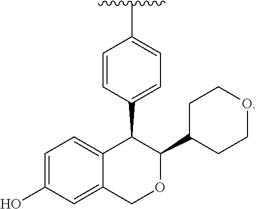 [SMILES: *c1ccc([C@@H]2c3ccc(O)cc3CO[C@@H]2C2CCOCC2)cc1]

In some embodiments, ERBM is

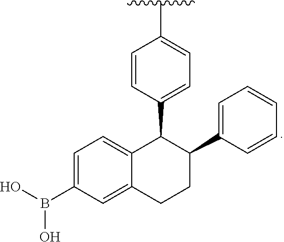 [SMILES: *c1ccc([C@@H]2c3ccc(B(O)O)cc3CC[C@@H]2c2ccccc2)cc1]

In some embodiments, ERBM is

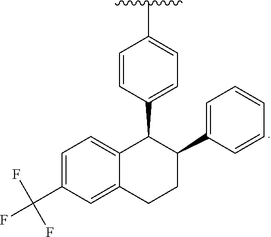 [SMILES: *c1ccc([C@@H]2c3ccc(C(F)(F)F)cc3CC[C@@H]2c2ccccc2)cc1]

In some embodiments, ERBM is

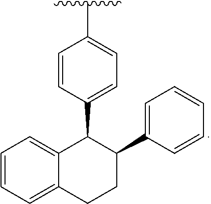 [SMILES: *c1ccc([C@@H]2c3ccccc3CC[C@@H]2c2ccccc2)cc1]

In some embodiments, ERBM is

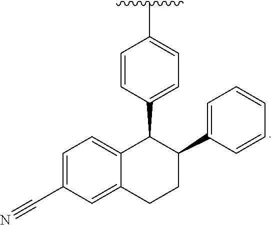 [SMILES: *c1ccc([C@@H]2c3ccc(C#N)cc3CC[C@@H]2c2ccccc2)cc1]

In some embodiments, ERBM is

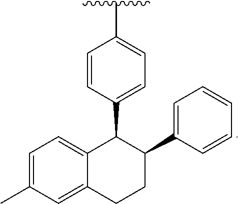 [SMILES: *c1ccc([C@@H]2c3ccc(C)cc3CC[C@@H]2c2ccccc2)cc1]

In some embodiments, ERBM is

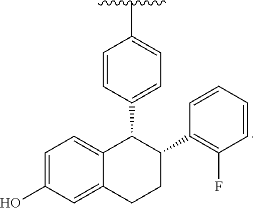 [SMILES: *c1ccc([C@H]2c3ccc(O)cc3CC[C@H]2c2ccccc2F)cc1]

In some embodiments, ERBM is

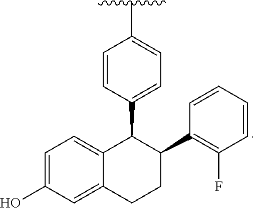 [SMILES: *c1ccc([C@@H]2c3ccc(O)cc3CC[C@@H]2c2ccccc2F)cc1]

In some embodiments, ERBM is

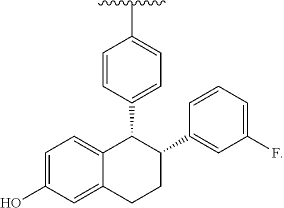 [SMILES: *c1ccc([C@H]2c3ccc(O)cc3CC[C@H]2c2cccc(F)c2)cc1]

In some embodiments, ERBM is

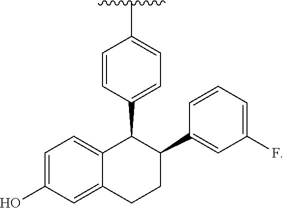 [SMILES: *c1ccc([C@@H]2c3ccc(O)cc3CC[C@@H]2c2cccc(F)c2)cc1]

In some embodiments, ERBM is

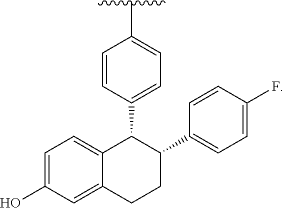 [SMILES: *c1ccc([C@H]2c3ccc(O)cc3CC[C@H]2c2ccc(F)cc2)cc1]

In some embodiments, ERBM is

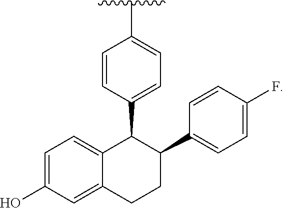 [SMILES: *c1ccc([C@@H]2c3ccc(O)cc3CC[C@@H]2c2ccc(F)cc2)cc1]

In some embodiments, ERBM is

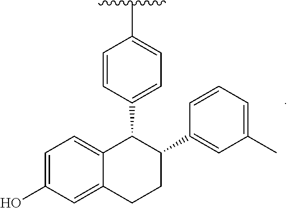 [SMILES: *c1ccc([C@H]2c3ccc(O)cc3CC[C@H]2c2cccc(C)c2)cc1 ; "."]

In some embodiments, ERBM is

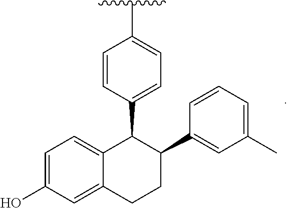 [SMILES: *c1ccc([C@@H]2c3ccc(O)cc3CC[C@@H]2c2cccc(C)c2)cc1 ; "."]

In some embodiments, ERBM is

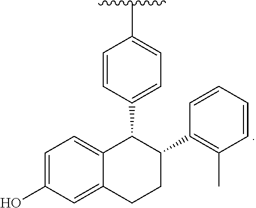 [SMILES: *c1ccc([C@H]2c3ccc(O)cc3CC[C@H]2c2ccccc2C)cc1]

In some embodiments, ERBM is

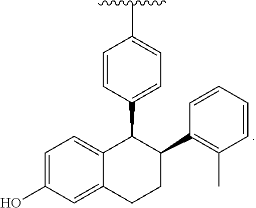 [SMILES: *c1ccc([C@@H]2c3ccc(O)cc3CC[C@@H]2c2ccccc2C)cc1]

In some embodiments, ERBM is

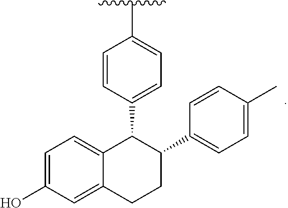 [SMILES: *c1ccc([C@H]2c3ccc(O)cc3CC[C@H]2c2ccc(C)cc2)cc1 ; "."]

In some embodiments, ERBM is

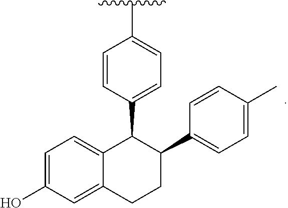 [SMILES: *c1ccc([C@@H]2c3ccc(O)cc3CC[C@@H]2c2ccc(C)cc2)cc1 ; "."]

In some embodiments, ERBM is

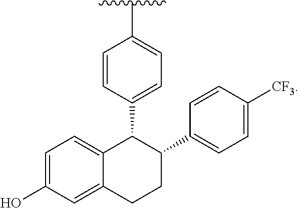 [SMILES: *c1ccc([C@H]2c3ccc(O)cc3CC[C@H]2c2ccc(C(F)(F)F)cc2)cc1]

In some embodiments, ERBM is

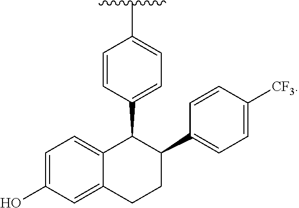 [SMILES: *c1ccc([C@@H]2c3ccc(O)cc3CC[C@@H]2c2ccc(C(F)(F)F)cc2)cc1]

In some embodiments, ERBM is

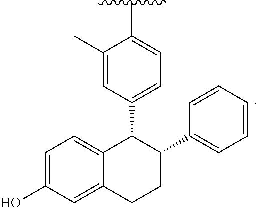 [SMILES: *c1ccc([C@H]2c3ccc(O)cc3CC[C@H]2c2ccccc2)cc1C ; "."]

In some embodiments, ERBM is

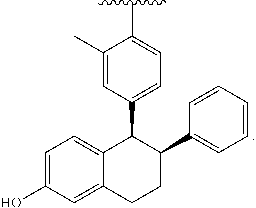 [SMILES: *c1ccc([C@@H]2c3ccc(O)cc3CC[C@@H]2c2ccccc2)cc1C]

In some embodiments, ERBM is

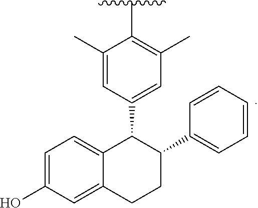 [SMILES: *c1c(C)cc([C@H]2c3ccc(O)cc3CC[C@H]2c2ccccc2)cc1C ; "."]

In some embodiments, ERBM is

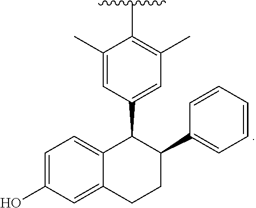 [SMILES: *c1c(C)cc([C@@H]2c3ccc(O)cc3CC[C@@H]2c2ccccc2)cc1C]

In some embodiments, ERBM is

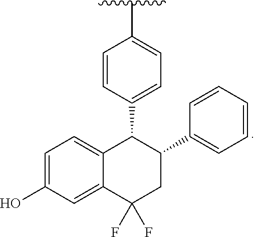 [SMILES: *c1ccc([C@H]2c3ccc(O)cc3C(F)(F)C[C@H]2c2ccccc2)cc1]

In some embodiments, ERBM is

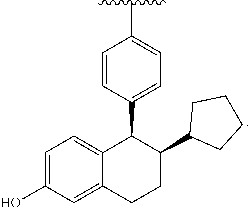 [SMILES: *c1ccc([C@@H]2c3ccc(O)cc3CC[C@@H]2C2CCCC2)cc1]

In some embodiments, ERBM is

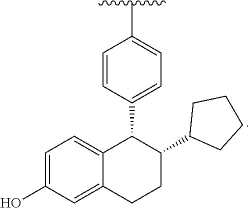 [SMILES: *c1ccc([C@H]2c3ccc(O)cc3CC[C@H]2C2CCCC2)cc1]

In some embodiments, ERBM is

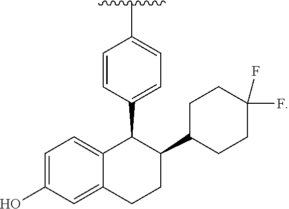 [SMILES: *c1ccc([C@@H]2c3ccc(O)cc3CC[C@@H]2C2CCC(F)(F)CC2)cc1]

In some embodiments, ERBM is

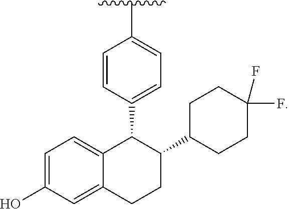 [SMILES: *c1ccc([C@H]2c3ccc(O)cc3CC[C@H]2C2CCC(F)(F)CC2)cc1]

In some embodiments, ERBM is

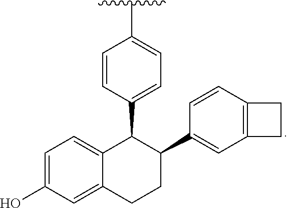 [SMILES: *c1ccc([C@@H]2c3ccc(O)cc3CC[C@@H]2c2ccc3c(c2)CC3)cc1]

In some embodiments, ERBM is

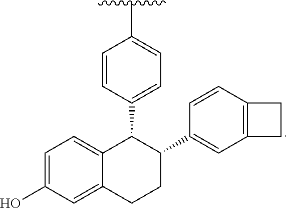 [SMILES: *c1ccc([C@H]2c3ccc(O)cc3CC[C@H]2c2ccc3c(c2)CC3)cc1]

In some embodiments, ERBM is

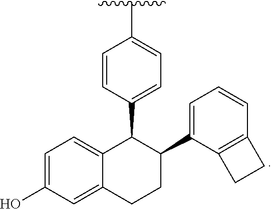 [SMILES: *c1ccc([C@@H]2c3ccc(O)cc3CC[C@@H]2c2cccc3c2CC3)cc1]

In some embodiments, ERBM is

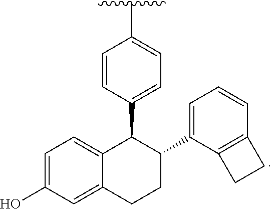 [SMILES: *c1ccc([C@@H]2c3ccc(O)cc3CC[C@H]2c2cccc3c2CC3)cc1]

In some embodiments, ERBM is

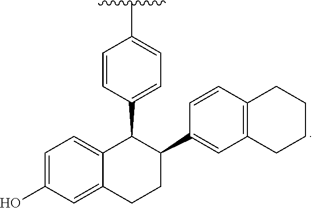 [SMILES: *c1ccc([C@@H]2c3ccc(O)cc3CC[C@@H]2c2ccc3c(c2)CCCC3)cc1]

In some embodiments, ERBM is

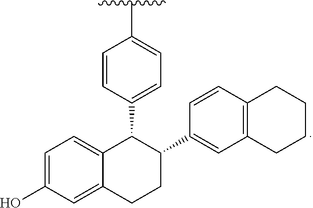 [SMILES: *c1ccc([C@H]2c3ccc(O)cc3CC[C@H]2c2ccc3c(c2)CCCC3)cc1]

In some embodiments, ERBM is

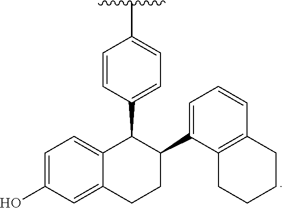 [SMILES: *c1ccc([C@@H]2c3ccc(O)cc3CC[C@@H]2c2cccc3c2CCCC3)cc1]

In some embodiments, ERBM is

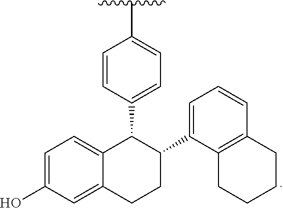 [SMILES: *c1ccc([C@H]2c3ccc(O)cc3CC[C@H]2c2cccc3c2CCCC3)cc1]

In some embodiments, ERBM is

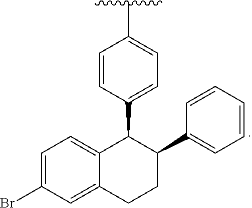 [SMILES: *c1ccc([C@@H]2c3ccc(Br)cc3CC[C@@H]2c2ccccc2)cc1]

In some embodiments, ERBM is

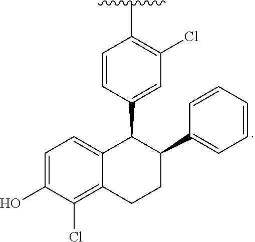 [SMILES: *c1ccc([C@@H]2c3ccc(O)c(Cl)c3CC[C@@H]2c2ccccc2)cc1Cl]

In some embodiments, ERBM is

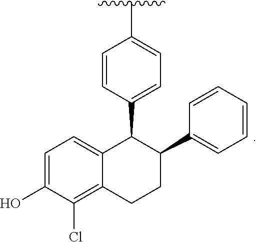 [SMILES: *c1ccc([C@@H]2c3ccc(O)c(Cl)c3CC[C@@H]2c2ccccc2)cc1]

In some embodiments, ERBM is

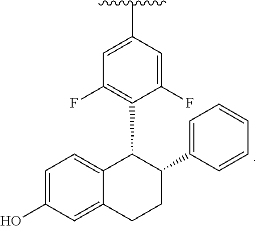 [SMILES: *c1cc(F)c([C@H]2c3ccc(O)cc3CC[C@H]2c2ccccc2)c(F)c1]

In some embodiments, ERBM is

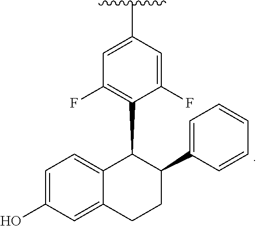 [SMILES: *c1cc(F)c([C@@H]2c3ccc(O)cc3CC[C@@H]2c2ccccc2)c(F)c1]

In some embodiments, ERBM is

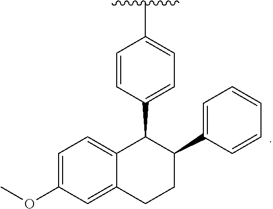 [SMILES: *c1ccc([C@@H]2c3ccc(OC)cc3CC[C@@H]2c2ccccc2)cc1]

In some embodiments, ERBM is

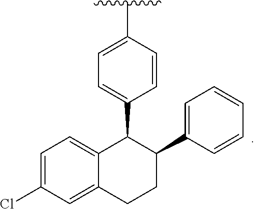 [SMILES: *c1ccc([C@@H]2c3ccc(Cl)cc3CC[C@@H]2c2ccccc2)cc1]

In some embodiments, ERBM is

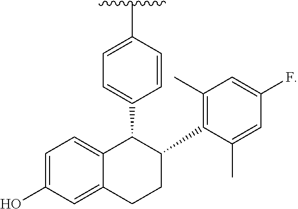 [SMILES: *c1ccc([C@H]2c3ccc(O)cc3CC[C@H]2c2c(C)cc(F)cc2C)cc1]

In some embodiments, ERBM is

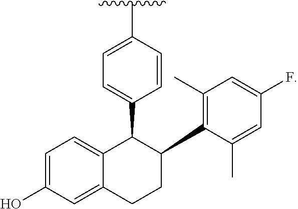 [SMILES: *c1ccc([C@@H]2c3ccc(O)cc3CC[C@@H]2c2c(C)cc(F)cc2C)cc1]

In some embodiments, ERBM is

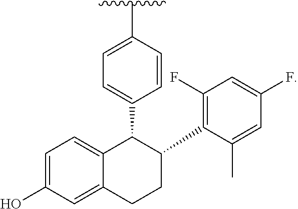 [SMILES: *c1ccc([C@H]2c3ccc(O)cc3CC[C@H]2c2c(C)cc(F)cc2F)cc1]

In some embodiments, ERBM is

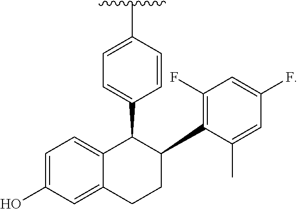 [SMILES: *c1ccc([C@@H]2c3ccc(O)cc3CC[C@@H]2c2c(C)cc(F)cc2F)cc1]

In some embodiments, ERBM is

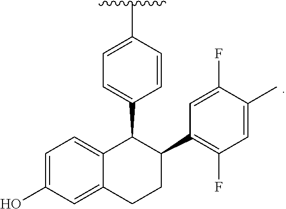 [SMILES: *c1ccc([C@@H]2c3ccc(O)cc3CC[C@@H]2c2cc(F)c(C)cc2F)cc1]

In some embodiments, ERBM is

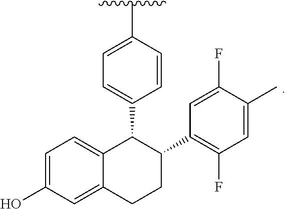 [SMILES: *c1ccc([C@H]2c3ccc(O)cc3CC[C@H]2c2cc(F)c(C)cc2F)cc1]

In some embodiments, ERBM is

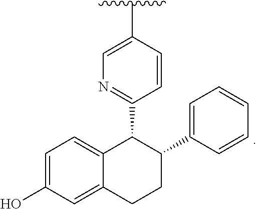 [SMILES: *c1ccc([C@H]2c3ccc(O)cc3CC[C@H]2c2ccccc2)nc1]

In some embodiments, ERBM is

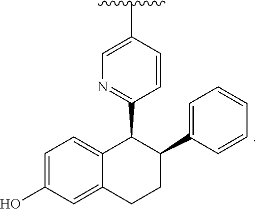 [SMILES: *c1ccc([C@@H]2c3ccc(O)cc3CC[C@@H]2c2ccccc2)nc1]

In some embodiments, ERBM is

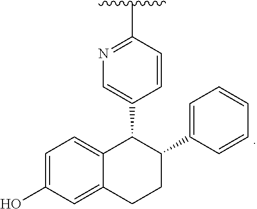 [SMILES: *c1ccc([C@H]2c3ccc(O)cc3CC[C@H]2c2ccccc2)cn1]

In some embodiments, ERBM is

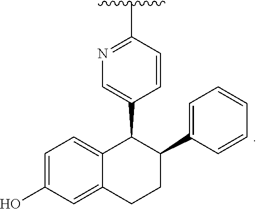 [SMILES: *c1ccc([C@@H]2c3ccc(O)cc3CC[C@@H]2c2ccccc2)cn1]

In some embodiments, ERBM is

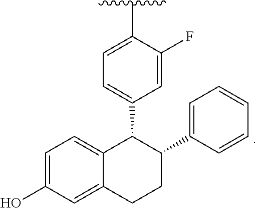 [SMILES: *c1ccc([C@H]2c3ccc(O)cc3CC[C@H]2c2ccccc2)cc1F]

In some embodiments, ERBM is

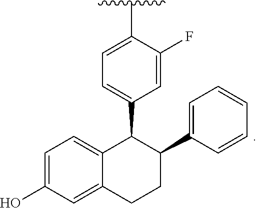 [SMILES: *c1ccc([C@@H]2c3ccc(O)cc3CC[C@@H]2c2ccccc2)cc1F]

In some embodiments, ERBM is

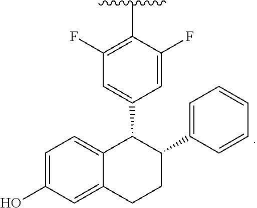 [SMILES: *c1c(F)cc([C@H]2c3ccc(O)cc3CC[C@H]2c2ccccc2)cc1F]

In some embodiments, ERBM is

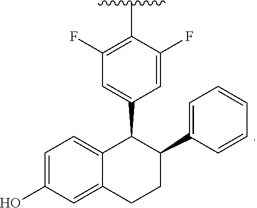 [SMILES: *c1c(F)cc([C@@H]2c3ccc(O)cc3CC[C@@H]2c2ccccc2)cc1F]

In some embodiments, ERBM is

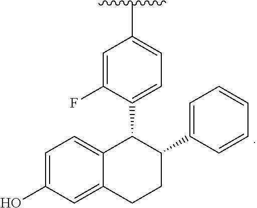 [SMILES: *c1ccc([C@H]2c3ccc(O)cc3CC[C@H]2c2ccccc2)c(F)c1]

In some embodiments, ERBM is

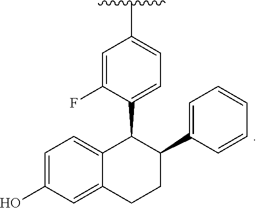 [SMILES: *c1ccc([C@@H]2c3ccc(O)cc3CC[C@@H]2c2ccccc2)c(F)c1]

In some embodiments, ERBM is

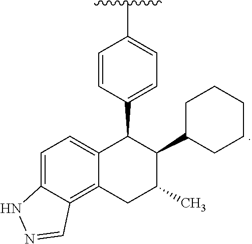 [SMILES: *c1ccc([C@@H]2c3ccc4[nH]ncc4c3C[C@@H](C)[C@@H]2C2CCCCC2)cc1]

In some embodiments, ERBM is

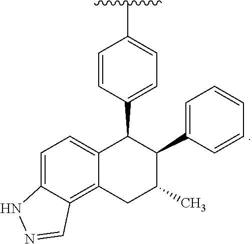 [SMILES: *c1ccc([C@@H]2c3ccc4[nH]ncc4c3C[C@@H](C)[C@@H]2c2ccccc2)cc1]

In some embodiments, ERBM is

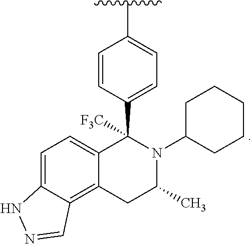 [SMILES: *c1ccc([C@]2(C(F)(F)F)c3ccc4[nH]ncc4c3C[C@@H](C)N2C2CCCCC2)cc1]

In some embodiments, ERBM is

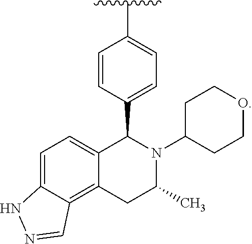 [SMILES: *c1ccc([C@@H]2c3ccc4[nH]ncc4c3C[C@@H](C)N2C2CCOCC2)cc1]

In some embodiments, ERBM is

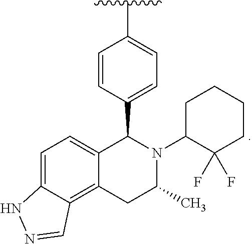 [SMILES: *c1ccc([C@@H]2c3ccc4[nH]ncc4c3C[C@@H](C)N2C2CCCCC2(F)F)cc1]

In some embodiments, ERBM is

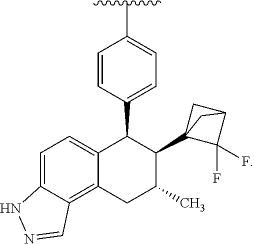 [SMILES: *c1ccc([C@@H]2c3ccc4[nH]ncc4c3C[C@@H](C)[C@@H]2C23CC(C2)C3(F)F)cc1]

In some embodiments, ERBM is

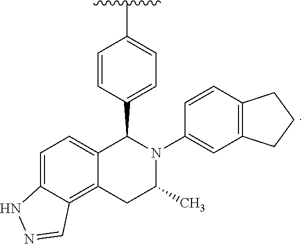 [SMILES: *c1ccc([C@@H]2c3ccc4[nH]ncc4c3C[C@@H](C)N2c2ccc3c(c2)CCC3)cc1]

In some embodiments, ERBM is

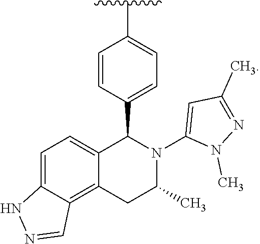 [SMILES: *c1ccc([C@@H]2c3ccc4[nH]ncc4c3C[C@@H](C)N2c2cc(C)nn2C)cc1]

In some embodiments, ERBM is

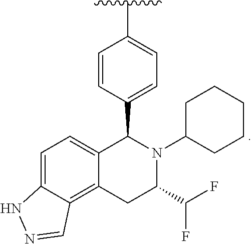 [SMILES: *c1ccc([C@@H]2c3ccc4[nH]ncc4c3C[C@@H](C(F)F)N2C2CCCCC2)cc1]

In some embodiments, ERBM is

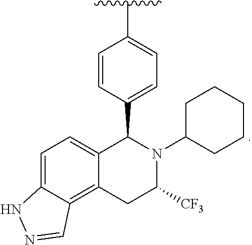 [SMILES: *c1ccc([C@@H]2c3ccc4[nH]ncc4c3C[C@@H](C(F)(F)F)N2C2CCCCC2)cc1]

In some embodiments, ERBM is

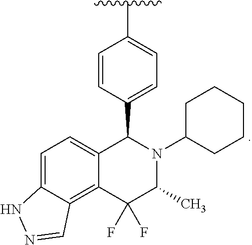 [SMILES: *c1ccc([C@@H]2c3ccc4[nH]ncc4c3C(F)(F)[C@@H](C)N2C2CCCCC2)cc1]

In some embodiments, ERBM is

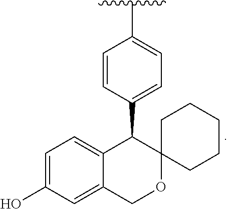 [SMILES: *c1ccc([C@@H]2c3ccc(O)cc3COC23CCCCC3)cc1]

In some embodiments, ERBM is

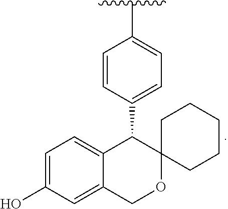 [SMILES: *c1ccc([C@H]2c3ccc(O)cc3COC23CCCCC3)cc1]

In some embodiments, ERBM is

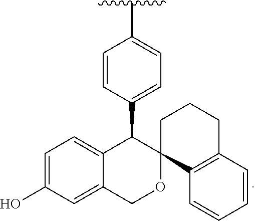 [SMILES: *c1ccc([C@@H]2c3ccc(O)cc3CO[C@@]23CCCc2ccccc23)cc1]

In some embodiments, ERBM is

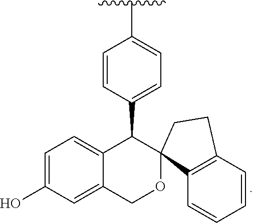 [SMILES: *c1ccc([C@@H]2c3ccc(O)cc3CO[C@@]23CCc2ccccc23)cc1]

In some embodiments, ERBM is

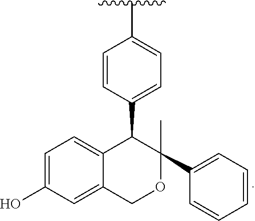 [SMILES: *c1ccc([C@@H]2c3ccc(O)cc3CO[C@]2(C)c2ccccc2)cc1]

In some embodiments, ERBM is

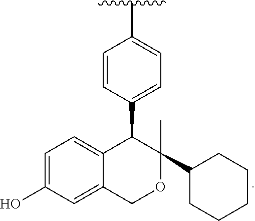 [SMILES: *c1ccc([C@@H]2c3ccc(O)cc3CO[C@]2(C)C2CCCCC2)cc1]

In some embodiments, ERBM is In some embodiments, ERBM is

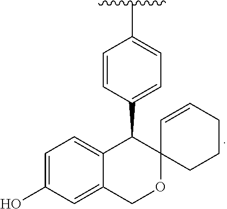 [SMILES: *c1ccc([C@@H]2c3ccc(O)cc3COC23C=CCCC3)cc1]

In some embodiments, ERBM is

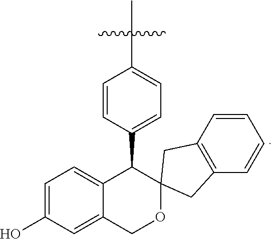 [SMILES: *c1ccc([C@@H]2c3ccc(O)cc3COC23Cc2ccccc2C3)cc1]

In some embodiments, ERBM is

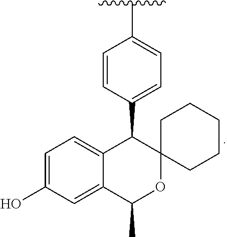 [SMILES: *c1ccc([C@@H]2c3ccc(O)cc3[C@H](C)OC23CCCCC3)cc1]

In some embodiments, ERBM is

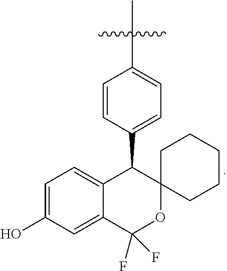 [SMILES: *c1ccc([C@@H]2c3ccc(O)cc3C(F)(F)OC23CCCCC3)cc1]

In some embodiments, ERBM is

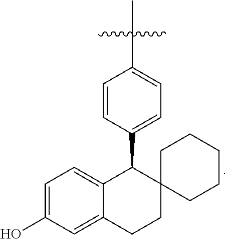 [SMILES: *c1ccc([C@@H]2c3ccc(O)cc3CCC23CCCCC3)cc1]

In some embodiments, ERBM is

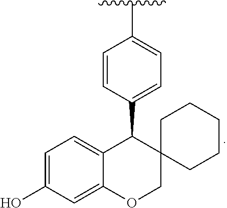 [SMILES: *c1ccc([C@@H]2c3ccc(O)cc3OCC23CCCCC3)cc1]

In some embodiments, ERBM is

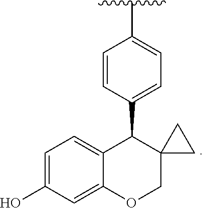 [SMILES: *c1ccc([C@@H]2c3ccc(O)cc3OCC23CC3)cc1]

In some embodiments, ERBM is

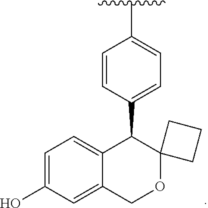 [SMILES: *c1ccc([C@@H]2c3ccc(O)cc3COC23CCC3)cc1]

In some embodiments, ERBM is

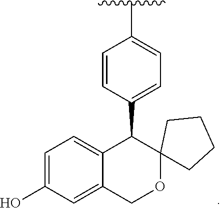 [SMILES: *c1ccc([C@@H]2c3ccc(O)cc3COC23CCCC3)cc1]

In some embodiments, ERBM is

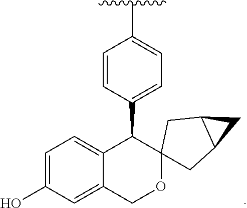 [SMILES: *c1ccc([C@@H]2c3ccc(O)cc3COC23C[C@H]2C[C@H]2C3)cc1]

In some embodiments, ERBM is

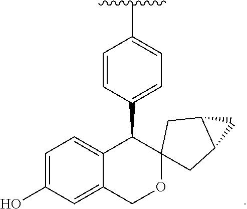 [SMILES: *c1ccc([C@@H]2c3ccc(O)cc3COC23C[C@H]2C[C@H]2C3)cc1]

In some embodiments, ERBM is

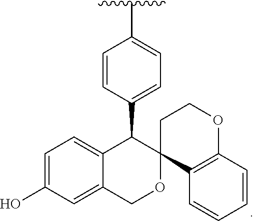 [SMILES: *c1ccc([C@@H]2c3ccc(O)cc3CO[C@@]23CCOc2ccccc23)cc1]

In some embodiments, ERBM is

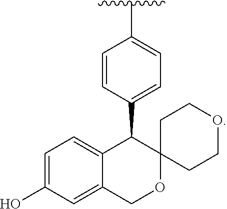 [SMILES: *c1ccc([C@@H]2c3ccc(O)cc3COC23CCOCC3)cc1]

In some embodiments, ERBM is

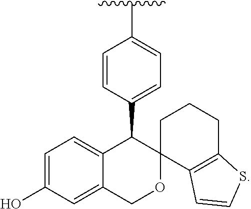 [SMILES: *c1ccc([C@@H]2c3ccc(O)cc3COC23CCCc2sccc23)cc1]

In some embodiments, ERBM is

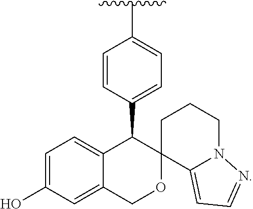 [SMILES: *c1ccc([C@@H]2c3ccc(O)cc3COC23CCCn2nccc23)cc1]

In some embodiments, ERBM is

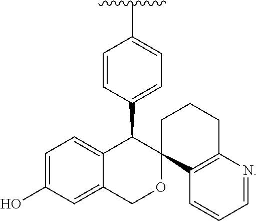 [SMILES: *c1ccc([C@@H]2c3ccc(O)cc3CO[C@@]23CCCc2ncccc23)cc1]

In some embodiments, ERBM is

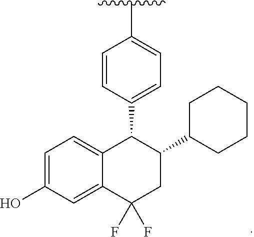 [SMILES: *c1ccc([C@H]2c3ccc(O)cc3C(F)(F)C[C@H]2C2CCCCC2)cc1]

In some embodiments, ERBM is

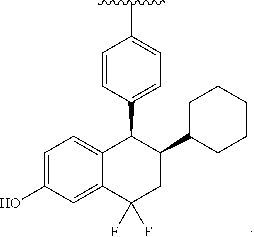 [SMILES: *c1ccc([C@@H]2c3ccc(O)cc3C(F)(F)C[C@@H]2C2CCCCC2)cc1]

In some embodiments, ERBM is

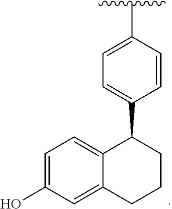 [SMILES: *c1ccc([C@H]2CCCc3cc(O)ccc32)cc1]

In some embodiments, ERBM is

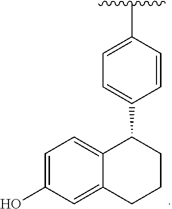 [SMILES: *c1ccc([C@@H]2CCCc3cc(O)ccc32)cc1]

In some embodiments, ERBM is

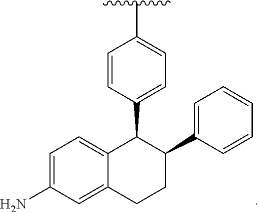 [SMILES: *c1ccc([C@@H]2c3ccc(N)cc3CC[C@@H]2c2ccccc2)cc1]

In some embodiments, ERBM is

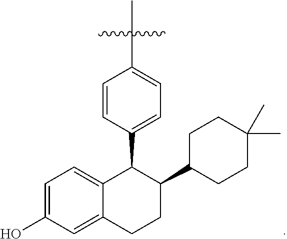 [SMILES: *c1ccc([C@@H]2c3ccc(O)cc3CC[C@@H]2C2CCC(C)(C)CC2)cc1]

In some embodiments, ERBM is

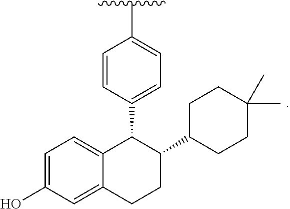 [SMILES: *c1ccc([C@H]2c3ccc(O)cc3CC[C@H]2C2CCC(C)(C)CC2)cc1]

In some embodiments, ERBM is

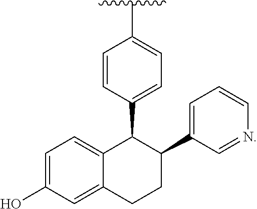 [SMILES: *c1ccc([C@@H]2c3ccc(O)cc3CC[C@@H]2c2cccnc2)cc1]

In some embodiments, ERBM is

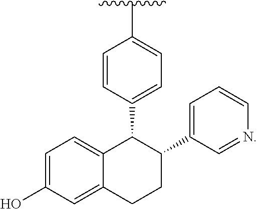 [SMILES: *c1ccc([C@H]2c3ccc(O)cc3CC[C@H]2c2cccnc2)cc1]

In some embodiments, ERBM is

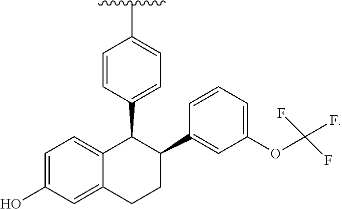 [SMILES: *c1ccc([C@@H]2c3ccc(O)cc3CC[C@@H]2c2cccc(OC(F)(F)F)c2)cc1]

In some embodiments, ERBM is

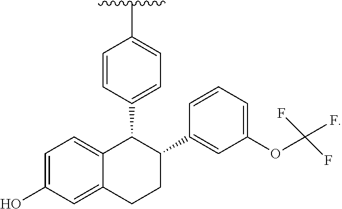 [SMILES: *c1ccc([C@H]2c3ccc(O)cc3CC[C@H]2c2cccc(OC(F)(F)F)c2)cc1]

In some embodiments, ERBM is

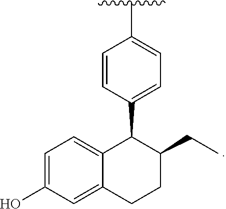 [SMILES: *c1ccc([C@@H]2c3ccc(O)cc3CC[C@@H]2CC)cc1]

In some embodiments, ERBM is

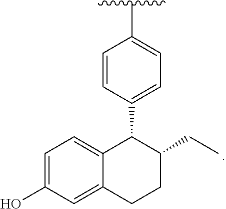 [SMILES: *c1ccc([C@H]2c3ccc(O)cc3CC[C@H]2CC)cc1]

In some embodiments, ERBM is

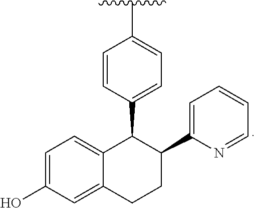 [SMILES: *c1ccc([C@@H]2c3ccc(O)cc3CC[C@@H]2c2ccccn2)cc1]

In some embodiments, ERBM is

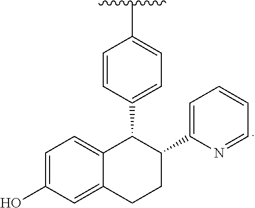 [SMILES: *c1ccc([C@H]2c3ccc(O)cc3CC[C@H]2c2ccccn2)cc1]

In some embodiments, ERBM is

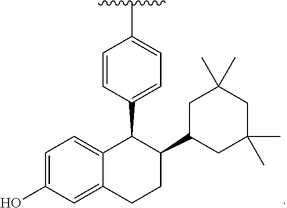 [SMILES: *c1ccc([C@@H]2c3ccc(O)cc3CC[C@@H]2C2CC(C)(C)CC(C)(C)C2)cc1]

In some embodiments, ERBM is

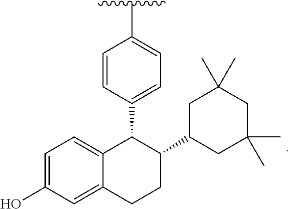 [SMILES: *c1ccc([C@H]2c3ccc(O)cc3CC[C@H]2C2CC(C)(C)CC(C)(C)C2)cc1]

In some embodiments, ERBM is

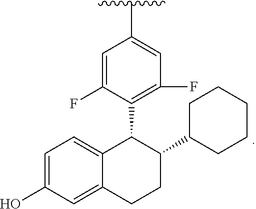 [SMILES: *c1cc(F)c([C@H]2c3ccc(O)cc3CC[C@H]2C2CCCCC2)c(F)c1]

In some embodiments, ERBM is

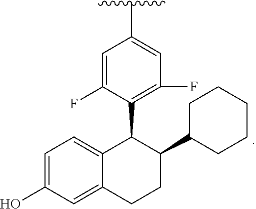 [SMILES: *c1cc(F)c([C@@H]2c3ccc(O)cc3CC[C@@H]2C2CCCCC2)c(F)c1]

In some embodiments, ERBM is

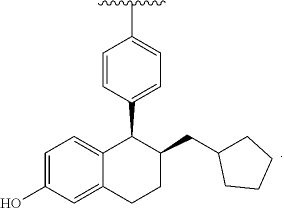 [SMILES: *c1ccc([C@@H]2c3ccc(O)cc3CC[C@@H]2CC2CCCC2)cc1]

In some embodiments, ERBM is

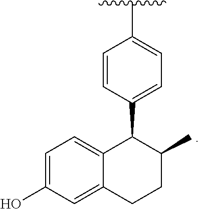 [SMILES: *c1ccc([C@@H]2c3ccc(O)cc3CC[C@@H]2C)cc1]

In some embodiments, ERBM is

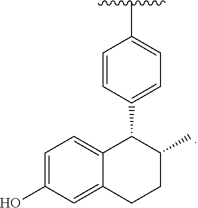 [SMILES: *c1ccc([C@H]2c3ccc(O)cc3CC[C@H]2C)cc1]

In some embodiments, ERBM is

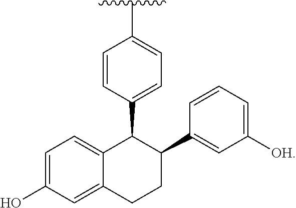 [SMILES: *c1ccc([C@@H]2c3ccc(O)cc3CC[C@@H]2c2cccc(O)c2)cc1]

In some embodiments, ERBM is

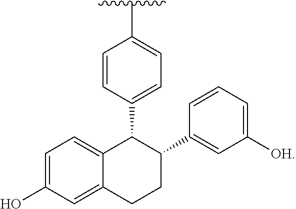 [SMILES: *c1ccc([C@H]2c3ccc(O)cc3CC[C@H]2c2cccc(O)c2)cc1]

In some embodiments, ERBM is

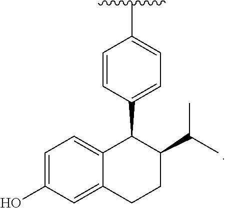 [SMILES: *c1ccc([C@@H]2c3ccc(O)cc3CC[C@@H]2C(C)C)cc1]

In some embodiments, ERBM is

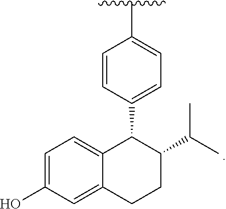 [SMILES: *c1ccc([C@H]2c3ccc(O)cc3CC[C@H]2C(C)C)cc1]

In some embodiments, ERBM is

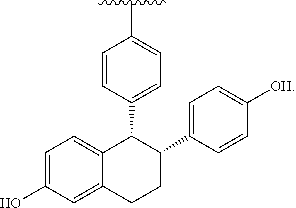 [SMILES: *c1ccc([C@H]2c3ccc(O)cc3CC[C@H]2c2ccc(O)cc2)cc1]

In some embodiments, ERBM is

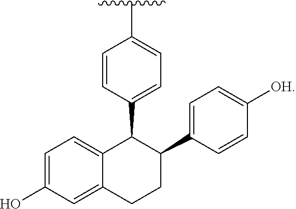 [SMILES: *c1ccc([C@@H]2c3ccc(O)cc3CC[C@@H]2c2ccc(O)cc2)cc1]

In some embodiments, ERBM is

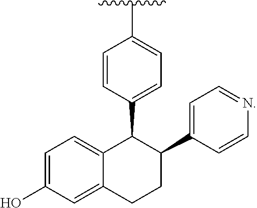 [SMILES: *c1ccc([C@@H]2c3ccc(O)cc3CC[C@@H]2c2ccncc2)cc1]

In some embodiments, ERBM is

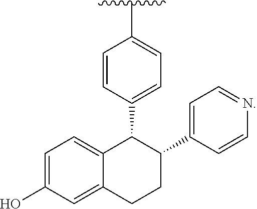 [SMILES: *c1ccc([C@H]2c3ccc(O)cc3CC[C@H]2c2ccncc2)cc1]

In some embodiments, ERBM is

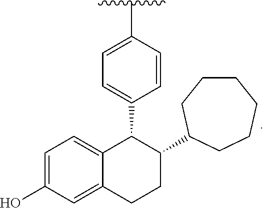 [SMILES: *c1ccc([C@H]2c3ccc(O)cc3CC[C@H]2C2CCCCCC2)cc1]

n some embodiments, ERBM is

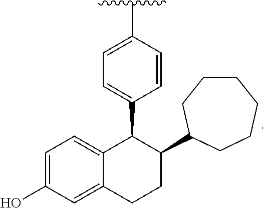 [SMILES: *c1ccc([C@@H]2c3ccc(O)cc3CC[C@@H]2C2CCCCCC2)cc1]

In some embodiments, ERBM is

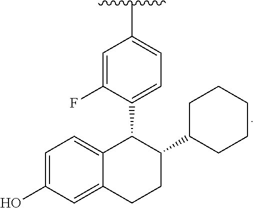 [SMILES: *c1ccc([C@H]2c3ccc(O)cc3CC[C@H]2C2CCCCC2)c(F)c1]

In some embodiments, ERBM is

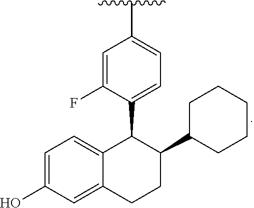 [SMILES: *c1ccc([C@@H]2c3ccc(O)cc3CC[C@@H]2C2CCCCC2)c(F)c1]

In some embodiments, ERBM is

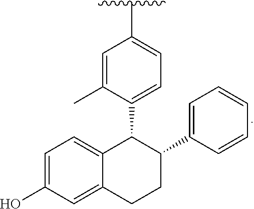 [SMILES: *c1ccc([C@H]2c3ccc(O)cc3CC[C@H]2c2ccccc2)c(C)c1]

In some embodiments, ERBM is

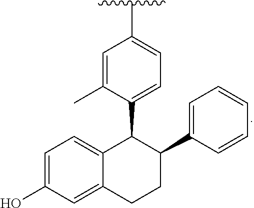 [SMILES: *c1ccc([C@@H]2c3ccc(O)cc3CC[C@@H]2c2ccccc2)c(C)c1]

In some embodiments, ERBM is

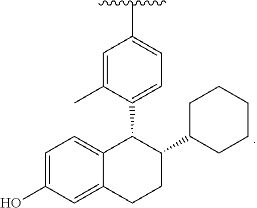 [SMILES: *c1ccc([C@H]2c3ccc(O)cc3CC[C@H]2C2CCCCC2)c(C)c1]

In some embodiments, ERBM is

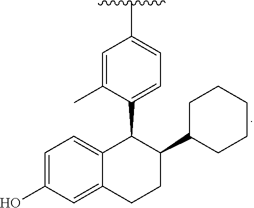 [SMILES: *c1ccc([C@@H]2c3ccc(O)cc3CC[C@@H]2C2CCCCC2)c(C)c1]

In some embodiments, ERBM is

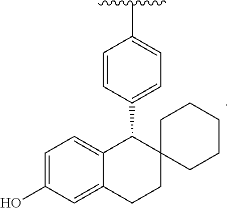 [SMILES: *c1ccc([C@H]2c3ccc(O)cc3CCC23CCCCC3)cc1]

In some embodiments, ERBM is

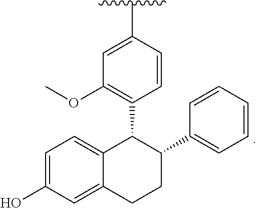 [SMILES: *c1ccc([C@H]2c3ccc(O)cc3CC[C@H]2c2ccccc2)c(OC)c1]

In some embodiments, ERBM is

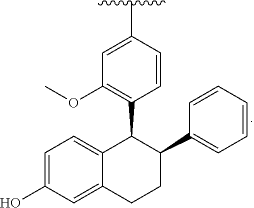 [SMILES: *c1ccc([C@@H]2c3ccc(O)cc3CC[C@@H]2c2ccccc2)c(OC)c1]

In some embodiments, ERBM is

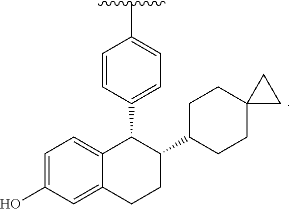 [SMILES: *c1ccc([C@H]2c3ccc(O)cc3CC[C@H]2C2CCC3(CC2)CC3)cc1]

In some embodiments, ERBM is

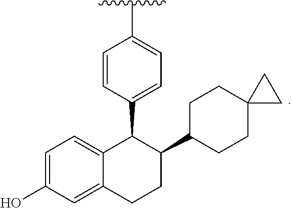 [SMILES: *c1ccc([C@@H]2c3ccc(O)cc3CC[C@@H]2C2CCC3(CC2)CC3)cc1]

In some embodiments, ERBM is

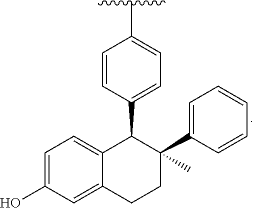 [SMILES: *c1ccc([C@@H]2c3ccc(O)cc3CC[C@]2(C)c2ccccc2)cc1]

In some embodiments, ERBM is

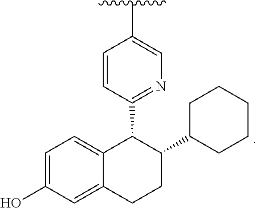 [SMILES: *c1ccc([C@H]2c3ccc(O)cc3CC[C@H]2C2CCCCC2)nc1]

In some embodiments, ERBM is

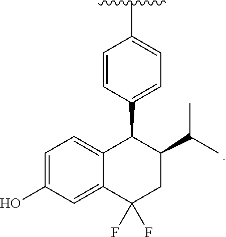 [SMILES: *c1ccc([C@@H]2c3ccc(O)cc3C(F)(F)C[C@@H]2C(C)C)cc1]

In some embodiments, ERBM is

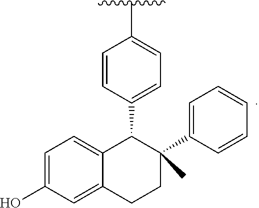 [SMILES: *c1ccc([C@H]2c3ccc(O)cc3CC[C@@]2(C)c2ccccc2)cc1 ; "."]

In some embodiments, ERBM is

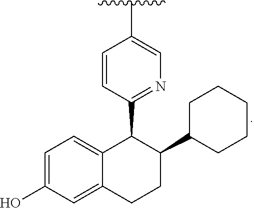 [SMILES: *c1ccc([C@@H]2c3ccc(O)cc3CC[C@@H]2C2CCCCC2)nc1]

In some embodiments, ERBM is

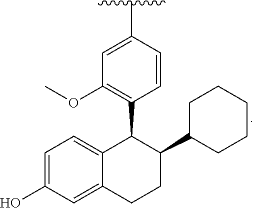 [SMILES: *c1ccc([C@@H]2c3ccc(O)cc3CC[C@@H]2C2CCCCC2)c(OC)c1]

In some embodiments, ERBM is

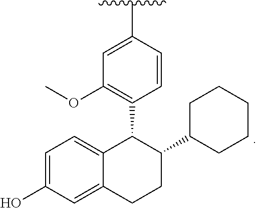 [SMILES: *c1ccc([C@H]2c3ccc(O)cc3CC[C@H]2C2CCCCC2)c(OC)c1]

In some embodiments, ERBM is

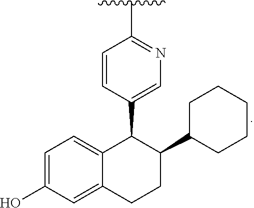 [SMILES: *c1ccc([C@@H]2c3ccc(O)cc3CC[C@@H]2C2CCCCC2)cn1]

In some embodiments, ERBM is

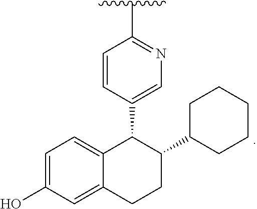 [SMILES: *c1ccc([C@H]2c3ccc(O)cc3CC[C@H]2C2CCCCC2)cn1]

In some embodiments, ERBM is

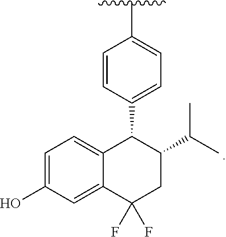 [SMILES: *c1ccc([C@H]2c3ccc(O)cc3C(F)(F)C[C@H]2C(C)C)cc1]

In some embodiments, ERBM is

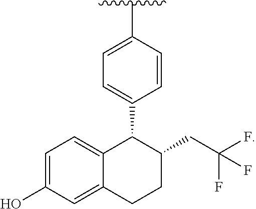 [SMILES: *c1ccc([C@H]2c3ccc(O)cc3CC[C@H]2CC(F)(F)F)cc1]

In some embodiments, ERBM is

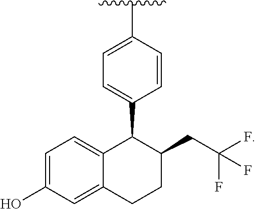 [SMILES: *c1ccc([C@@H]2c3ccc(O)cc3CC[C@@H]2CC(F)(F)F)cc1]

In some embodiments, ERBM is

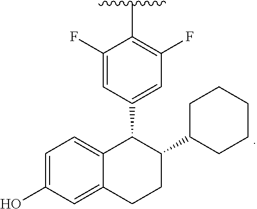 [SMILES: *c1c(F)cc([C@H]2c3ccc(O)cc3CC[C@H]2C2CCCCC2)cc1F]

In some embodiments, ERBM is

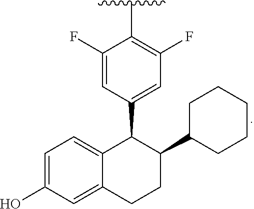 [SMILES: *c1c(F)cc([C@@H]2c3ccc(O)cc3CC[C@@H]2C2CCCCC2)cc1F]

In some embodiments, ERBM is

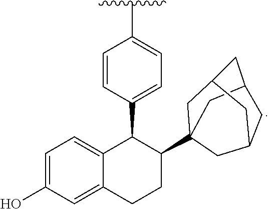 [SMILES: *c1ccc([C@@H]2c3ccc(O)cc3CC[C@@H]2C23CC4CC(CC(C4)C2)C3)cc1]

In some embodiments, ERBM is

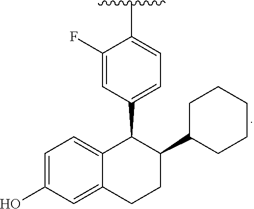 [SMILES: *c1ccc([C@@H]2c3ccc(O)cc3CC[C@@H]2C2CCCCC2)cc1F]

In some embodiments, ERBM is

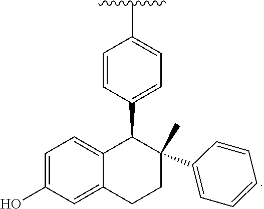 [SMILES: *c1ccc([C@@H]2c3ccc(O)cc3CC[C@@]2(C)c2ccccc2)cc1]

In some embodiments, ERBM is

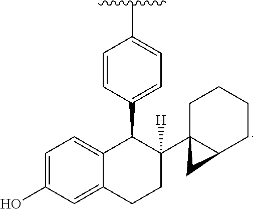 [SMILES: *c1ccc([C@@H]2c3ccc(O)cc3CC[C@@H]2[C@]23CCCC[C@H]2C3)cc1]

In some embodiments, ERBM is

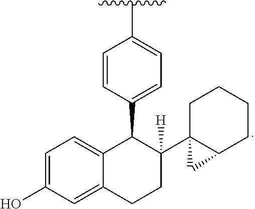 [SMILES: *c1ccc([C@@H]2c3ccc(O)cc3CC[C@@H]2[C@@]23CCCC[C@@H]2C3)cc1]

In some embodiments, ERBM is

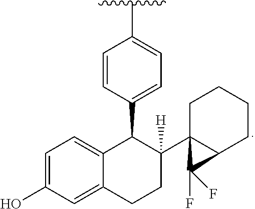 [SMILES: *c1ccc([C@@H]2c3ccc(O)cc3CC[C@@H]2[C@]23CCCC[C@H]2C3(F)F)cc1]

In some embodiments, ERBM is

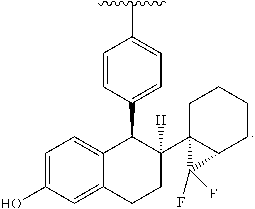 [SMILES: *c1ccc([C@@H]2c3ccc(O)cc3CC[C@@H]2[C@@]23CCCC[C@@H]2C3(F)F)cc1]

In some embodiments, ERBM is

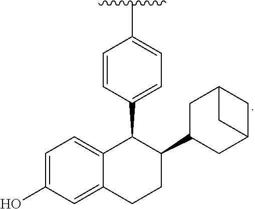 [SMILES: *c1ccc([C@@H]2c3ccc(O)cc3CC[C@@H]2C2CC3CC(C3)C2)cc1]

In some embodiments, ERBM is

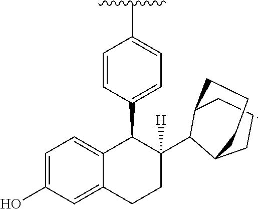 [SMILES: *c1ccc([C@@H]2c3ccc(O)cc3CC[C@@H]2C2[C@H]3CCC[C@@H]2CCC3)cc1]

In some embodiments, ERBM is

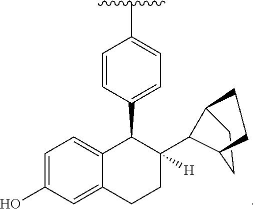 [SMILES: *c1ccc([C@@H]2c3ccc(O)cc3CC[C@@H]2C2[C@H]3CC[C@H]2CC3)cc1]

In some embodiments, ERBM is

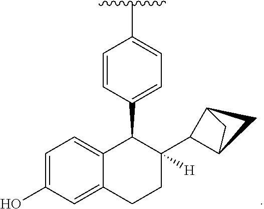 [SMILES: *c1ccc([C@@H]2c3ccc(O)cc3CC[C@@H]2C2[C@H]3C[C@@H]2C3)cc1]

In some embodiments, ERBM is

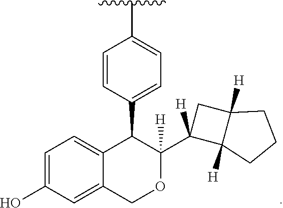 [SMILES: *c1ccc([C@@H]2c3ccc(O)cc3CO[C@@H]2[C@@H]2C[C@@H]3CCC[C@@H]32)cc1]

In some embodiments, ERBM is

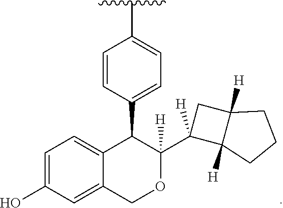 [SMILES: *c1ccc([C@@H]2c3ccc(O)cc3CO[C@@H]2[C@H]2C[C@@H]3CCC[C@@H]32)cc1]

In some embodiments, ERBM is

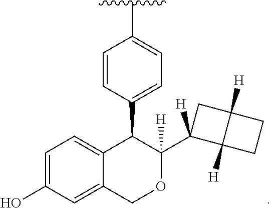 [SMILES: *c1ccc([C@@H]2c3ccc(O)cc3CO[C@@H]2[C@@H]2C[C@@H]3CC[C@@H]32)cc1]

In some embodiments, ERBM is

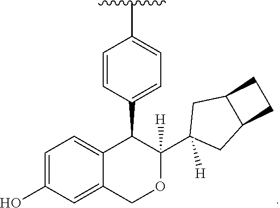

In some embodiments, ERBM is

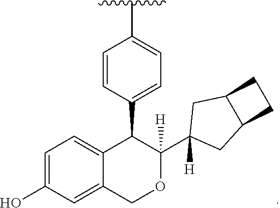 [SMILES: *c1ccc([C@@H]2c3ccc(O)cc3CO[C@@H]2[C@@H]2C[C@H]3CC[C@H]3C2)cc1]

In some embodiments, ERBM is

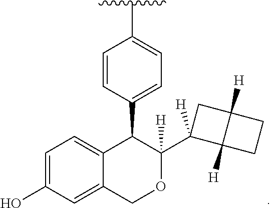 [SMILES: *c1ccc([C@@H]2c3ccc(O)cc3CO[C@@H]2[C@H]2C[C@@H]3CC[C@@H]32)cc1]

In some embodiments, ERBM is

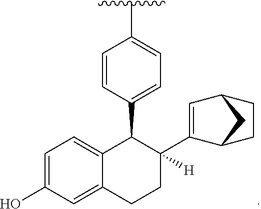 [SMILES: *c1ccc([C@@H]2c3ccc(O)cc3CC[C@@H]2C2=C[C@@H]3CC[C@H]2C3)cc1]

In some embodiments, ERBM is

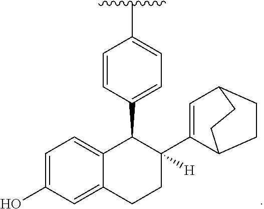 [SMILES: *c1ccc([C@@H]2c3ccc(O)cc3CC[C@@H]2C2=CC3CCC2CC3)cc1]

In some embodiments, ERBM is

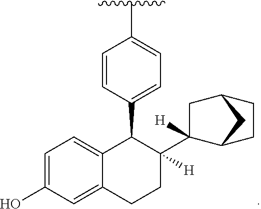 [SMILES: *c1ccc([C@@H]2c3ccc(O)cc3CC[C@@H]2[C@@H]2C[C@@H]3CC[C@H]2C3)cc1]

In some embodiments, ERBM is

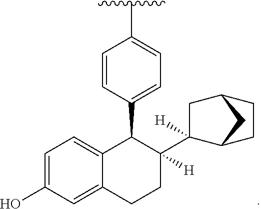 [SMILES: *c1ccc([C@@H]2c3ccc(O)cc3CC[C@@H]2[C@H]2C[C@@H]3CC[C@H]2C3)cc1]

In some embodiments, ERBM is

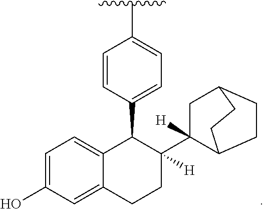 [SMILES: *c1ccc([C@@H]2c3ccc(O)cc3CC[C@@H]2[C@@H]2CC3CCC2CC3)cc1]

In some embodiments, ERBM is

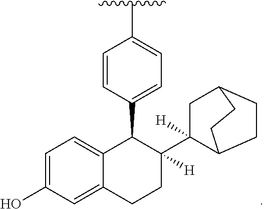 [SMILES: *c1ccc([C@@H]2c3ccc(O)cc3CC[C@@H]2[C@H]2CC3CCC2CC3)cc1]

In some embodiments, ERBM is

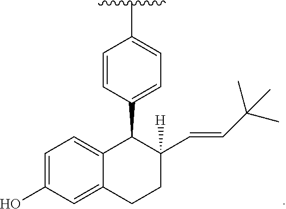 [SMILES: *c1ccc([C@@H]2c3ccc(O)cc3CC[C@@H]2/C=C/C(C)(C)C)cc1]

In some embodiments, ERBM is

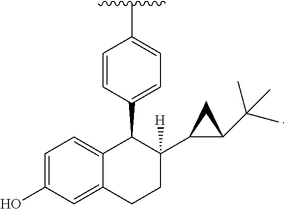 [SMILES: *c1ccc([C@@H]2c3ccc(O)cc3CC[C@@H]2[C@H]2C[C@@H]2C(C)(C)C)cc1]

In some embodiments, ERBM is

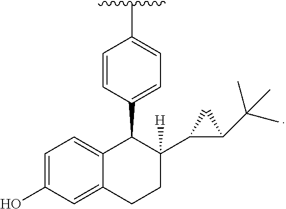 [SMILES: *c1ccc([C@@H]2c3ccc(O)cc3CC[C@@H]2[C@@H]2C[C@H]2C(C)(C)C)cc1]

In some embodiments, ERBM is

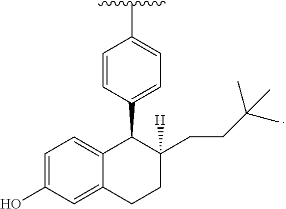 [SMILES: *c1ccc([C@@H]2c3ccc(O)cc3CC[C@@H]2CCC(C)(C)C)cc1]

In some embodiments, ERBM is

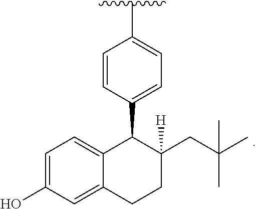 [SMILES: *c1ccc([C@@H]2c3ccc(O)cc3CC[C@@H]2CC(C)(C)C)cc1]

In some embodiments, ERBM is

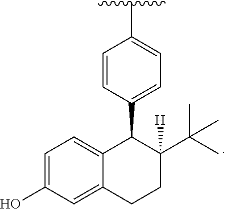 [SMILES: *c1ccc([C@@H]2c3ccc(O)cc3CC[C@@H]2C(C)(C)C)cc1]

In some embodiments, ERBM is

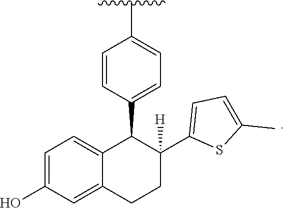 [SMILES: *c1ccc([C@@H]2c3ccc(O)cc3CC[C@@H]2c2ccc(C)s2)cc1]

In some embodiments, ERBM is

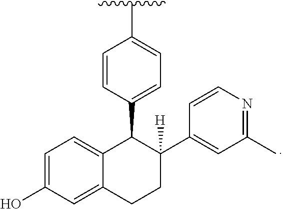 [SMILES: *c1ccc([C@@H]2c3ccc(O)cc3CC[C@@H]2c2ccnc(C)c2)cc1]

In some embodiments, ERBM is

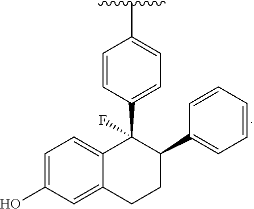 [SMILES: *c1ccc([C@]2(F)c3ccc(O)cc3CC[C@@H]2c2ccccc2)cc1]

In some embodiments, ERBM is

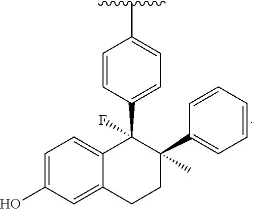 [SMILES: *c1ccc([C@]2(F)c3ccc(O)cc3CC[C@]2(C)c2ccccc2)cc1]

In some embodiments, ERBM is

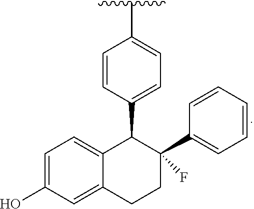 [SMILES: *c1ccc([C@@H]2c3ccc(O)cc3CC[C@]2(F)c2ccccc2)cc1]

In some embodiments, ERBM is

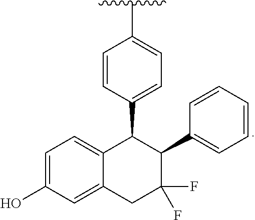 [SMILES: *c1ccc([C@@H]2c3ccc(O)cc3CC(F)(F)[C@@H]2c2ccccc2)cc1]

In some embodiments, ERBM is

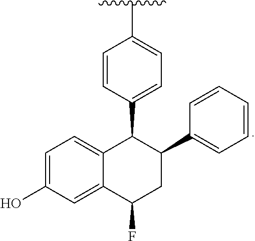 [SMILES: *c1ccc([C@@H]2c3ccc(O)cc3[C@H](F)C[C@@H]2c2ccccc2)cc1]

In some embodiments, ERBM is

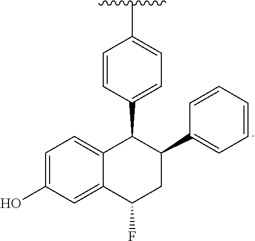 [SMILES: *c1ccc([C@@H]2c3ccc(O)cc3[C@@H](F)C[C@@H]2c2ccccc2)cc1]

In some embodiments, ERBM is

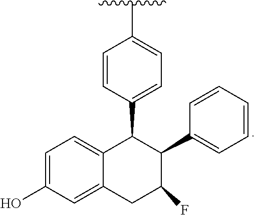 [SMILES: *c1ccc([C@@H]2c3ccc(O)cc3C[C@H](F)[C@@H]2c2ccccc2)cc1]

In some embodiments, ERBM is

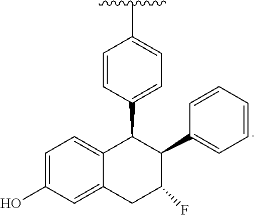 [SMILES: *c1ccc([C@@H]2c3ccc(O)cc3C[C@@H](F)[C@@H]2c2ccccc2)cc1]

In some embodiments, ERBM is

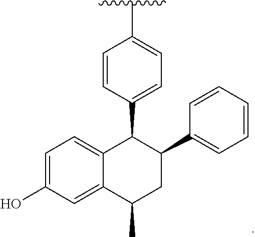 [SMILES: *c1ccc([C@@H]2c3ccc(O)cc3[C@H](C)C[C@@H]2c2ccccc2)cc1]

In some embodiments, ERBM is

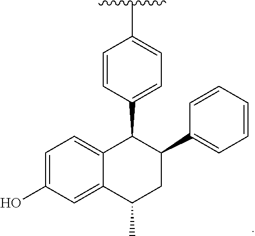 [SMILES: *c1ccc([C@@H]2c3ccc(O)cc3[C@@H](C)C[C@@H]2c2ccccc2)cc1]

In some embodiments, ERBM is

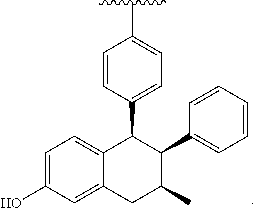 [SMILES: *c1ccc([C@@H]2c3ccc(O)cc3C[C@H](C)[C@@H]2c2ccccc2)cc1]

In some embodiments, ERBM is

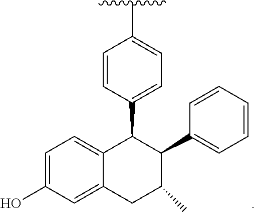 [SMILES: *c1ccc([C@@H]2c3ccc(O)cc3C[C@@H](C)[C@@H]2c2ccccc2)cc1]

In some embodiments, ERBM is

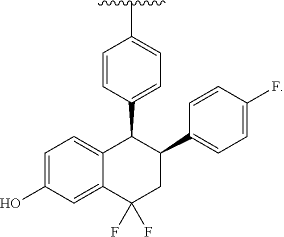 [SMILES: *c1ccc([C@@H]2c3ccc(O)cc3C(F)(F)C[C@@H]2c2ccc(F)cc2)cc1]

In some embodiments, ERBM is

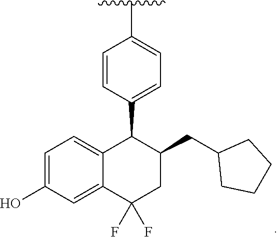 [SMILES: *c1ccc([C@@H]2c3ccc(O)cc3C(F)(F)C[C@@H]2CC2CCCC2)cc1]

In some embodiments, ERBM is

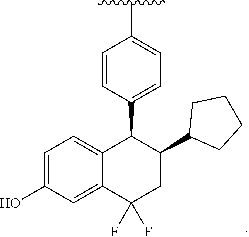 [SMILES: *c1ccc([C@@H]2c3ccc(O)cc3C(F)(F)C[C@@H]2C2CCCC2)cc1]

In some embodiments, ERBM is

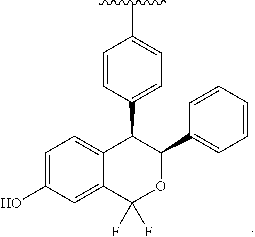 [SMILES: *c1ccc([C@@H]2c3ccc(O)cc3C(F)(F)O[C@@H]2c2ccccc2)cc1]

In some embodiments, ERBM is

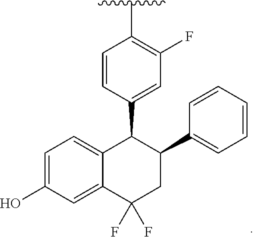 [SMILES: *c1ccc([C@@H]2c3ccc(O)cc3C(F)(F)C[C@@H]2c2ccccc2)cc1F]

In some embodiments, ERBM is

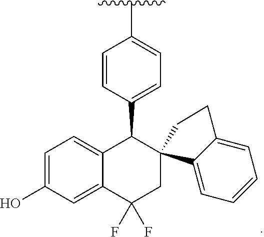 [SMILES: *c1ccc([C@@H]2c3ccc(O)cc3C(F)(F)C[C@@]23CCc2ccccc23)cc1]

In some embodiments, ERBM is

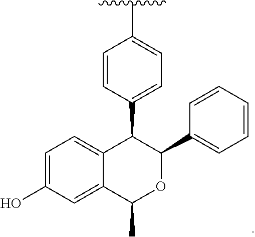 [SMILES: *c1ccc([C@@H]2c3ccc(O)cc3[C@H](C)O[C@@H]2c2ccccc2)cc1]

In some embodiments, ERBM is

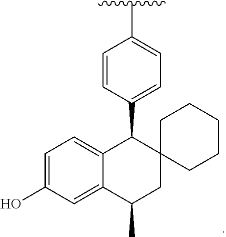 [SMILES: *c1ccc([C@@H]2c3ccc(O)cc3[C@H](C)CC23CCCCC3)cc1]

In some embodiments, ERBM is

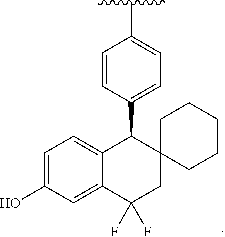 [SMILES: *c1ccc([C@@H]2c3ccc(O)cc3C(F)(F)CC23CCCCC3)cc1]

In some embodiments, ERBM is

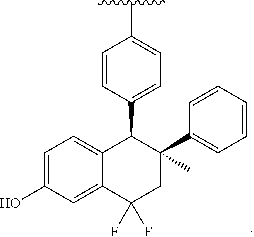 [SMILES: *c1ccc([C@@H]2c3ccc(O)cc3C(F)(F)C[C@]2(C)c2ccccc2)cc1]

In some embodiments, ERBM is

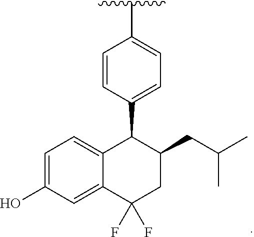 [SMILES: *c1ccc([C@@H]2c3ccc(O)cc3C(F)(F)C[C@@H]2CC(C)C)cc1]

In some embodiments, ERBM is

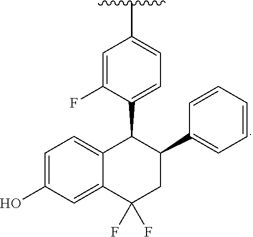 [SMILES: *c1ccc([C@@H]2c3ccc(O)cc3C(F)(F)C[C@@H]2c2ccccc2)c(F)c1]

In some embodiments, ERBM is

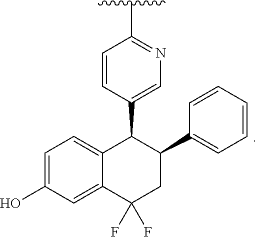 [SMILES: *c1ccc([C@@H]2c3ccc(O)cc3C(F)(F)C[C@@H]2c2ccccc2)cn1]

In some embodiments, ERBM is

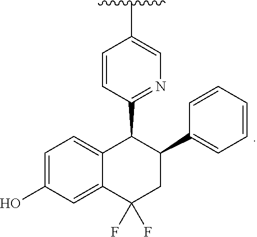 [SMILES: *c1ccc([C@@H]2c3ccc(O)cc3C(F)(F)C[C@@H]2c2ccccc2)nc1]

In some embodiments, ERBM is

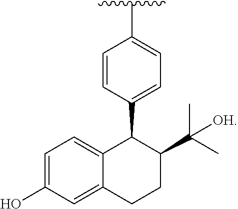 [SMILES: *c1ccc([C@@H]2c3ccc(O)cc3CC[C@@H]2C(C)(C)O)cc1]

In some embodiments, ERBM is

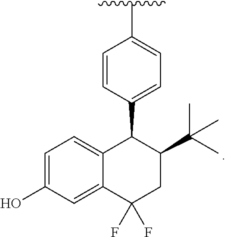 [SMILES: *c1ccc([C@@H]2c3ccc(O)cc3C(F)(F)C[C@@H]2C(C)(C)C)cc1]

In some embodiments, ERBM is

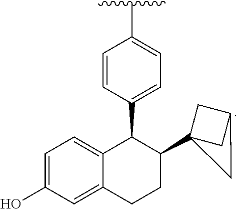 [SMILES: *c1ccc([C@@H]2c3ccc(O)cc3CC[C@@H]2C23CC(C2)C3)cc1]

In some embodiments, ERBM is

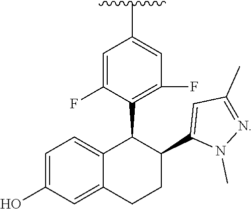 [SMILES: *c1cc(F)c([C@@H]2c3ccc(O)cc3CC[C@@H]2c2cc(C)nn2C)c(F)c1]

In some embodiments, ERBM is

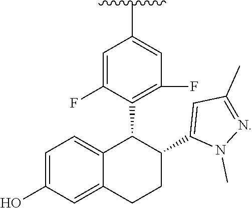 [SMILES: *c1cc(F)c([C@H]2c3ccc(O)cc3CC[C@H]2c2cc(C)nn2C)c(F)c1]

In some embodiments, ERBM is

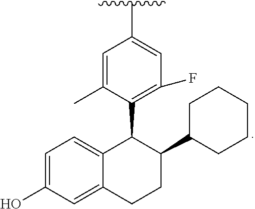 [SMILES: *c1cc(C)c([C@@H]2c3ccc(O)cc3CC[C@@H]2C2CCCCC2)c(F)c1]

In some embodiments, ERBM is

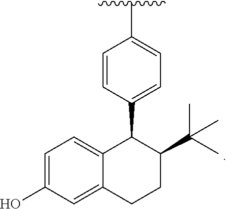 [SMILES: *c1ccc([C@@H]2c3ccc(O)cc3CC[C@@H]2C(C)(C)C)cc1]

In some embodiments, ERBM is

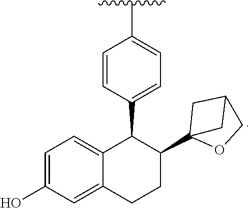 [SMILES: *c1ccc([C@@H]2c3ccc(O)cc3CC[C@@H]2C23CC(CO2)C3)cc1]

In some embodiments, ERBM is

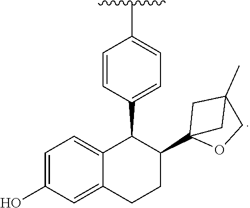 [SMILES: *c1ccc([C@@H]2c3ccc(O)cc3CC[C@@H]2C23CC(C)(CO2)C3)cc1]

In some embodiments, ERBM is

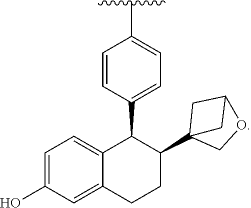 [SMILES: *c1ccc([C@@H]2c3ccc(O)cc3CC[C@@H]2C23COC(C2)C3)cc1]

In some embodiments, ERBM is

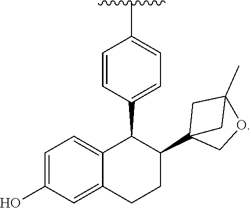 [SMILES: *c1ccc([C@@H]2c3ccc(O)cc3CC[C@@H]2C23COC(C)(C2)C3)cc1]

In some embodiments, ERBM is

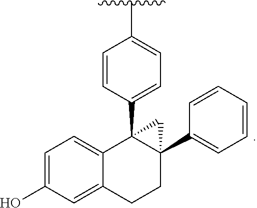 [SMILES: *c1ccc([C@@]23C[C@]2(c2ccccc2)CCc2cc(O)ccc23)cc1]

In some embodiments, ERBM is

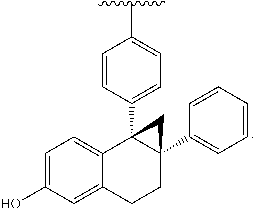 [SMILES: *c1ccc([C@]23C[C@@]2(c2ccccc2)CCc2cc(O)ccc23)cc1]

In some embodiments, ERBM is

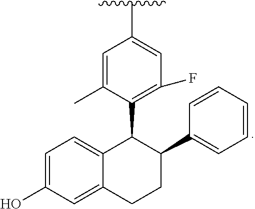 [SMILES: *c1cc(C)c([C@@H]2c3ccc(O)cc3CC[C@@H]2c2ccccc2)c(F)c1]

In some embodiments, ERBM is

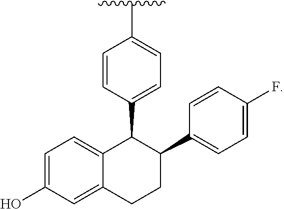 [SMILES: *c1ccc([C@@H]2c3ccc(O)cc3CC[C@@H]2c2ccc(F)cc2)cc1]

In some embodiments, ERBM is

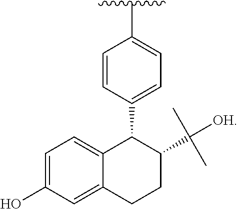 [SMILES: *c1ccc([C@H]2c3ccc(O)cc3CC[C@H]2C(C)(C)O)cc1]

In some embodiments, ERBM is

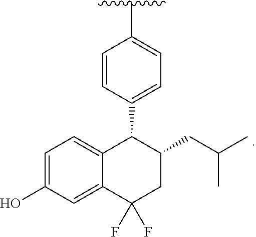 [SMILES: *c1ccc([C@H]2c3ccc(O)cc3C(F)(F)C[C@H]2CC(C)C)cc1]

In some embodiments, ERBM is

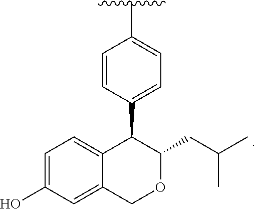 [SMILES: *c1ccc([C@@H]2c3ccc(O)cc3CO[C@H]2CC(C)C)cc1]

In some embodiments, ERBM is

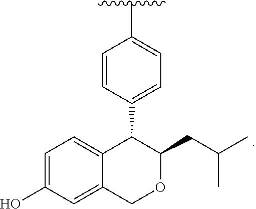 [SMILES: *c1ccc([C@H]2c3ccc(O)cc3CO[C@@H]2CC(C)C)cc1]

In some embodiments, ERBM is

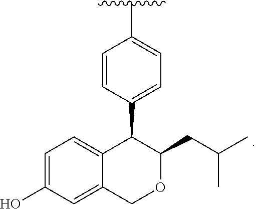 [SMILES: *c1ccc([C@@H]2c3ccc(O)cc3CO[C@@H]2CC(C)C)cc1]

In some embodiments, ERBM is

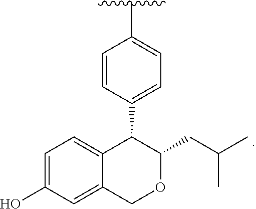 [SMILES: *c1ccc([C@H]2c3ccc(O)cc3CO[C@H]2CC(C)C)cc1]

In some embodiments, ERBM is

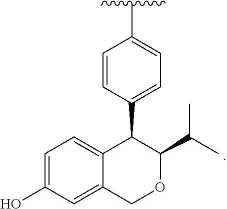 [SMILES: *c1ccc([C@@H]2c3ccc(O)cc3CO[C@@H]2C(C)C)cc1]

In some embodiments, ERBM is

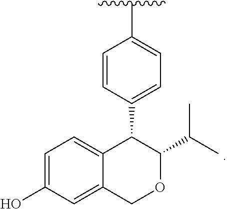 [SMILES: *c1ccc([C@H]2c3ccc(O)cc3CO[C@H]2C(C)C)cc1]

In some embodiments, ERBM is

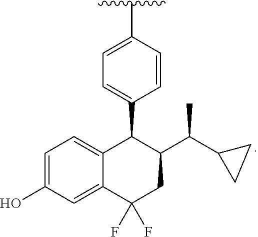 [SMILES: *c1ccc([C@@H]2c3ccc(O)cc3C(F)(F)C[C@@H]2[C@@H](C)C2CC2)cc1]

In some embodiments, ERBM is

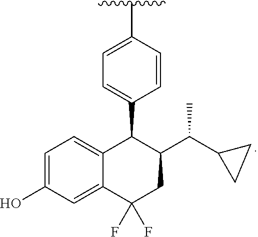 [SMILES: *c1ccc([C@@H]2c3ccc(O)cc3C(F)(F)C[C@@H]2[C@H](C)C2CC2)cc1]

In some embodiments, ERBM is

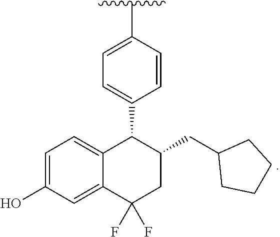 [SMILES: *c1ccc([C@H]2c3ccc(O)cc3C(F)(F)C[C@H]2CC2CCCC2)cc1]

In some embodiments, ERBM is

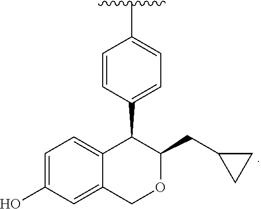 [SMILES: *c1ccc([C@@H]2c3ccc(O)cc3CO[C@@H]2CC2CC2)cc1]

In some embodiments, ERBM is

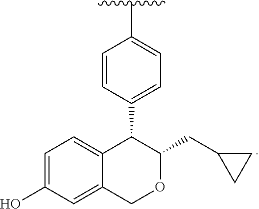 [SMILES: *c1ccc([C@H]2c3ccc(O)cc3CO[C@H]2CC2CC2)cc1]

In some embodiments, ERBM is

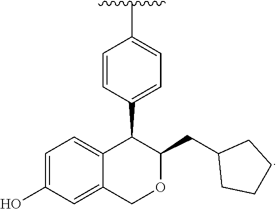 [SMILES: *c1ccc([C@@H]2c3ccc(O)cc3CO[C@@H]2CC2CCCC2)cc1]

In some embodiments, ERBM is

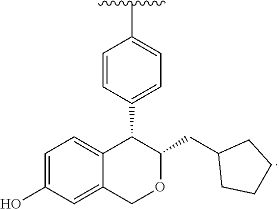 [SMILES: *c1ccc([C@H]2c3ccc(O)cc3CO[C@H]2CC2CCCC2)cc1]

In some embodiments, ERBM is

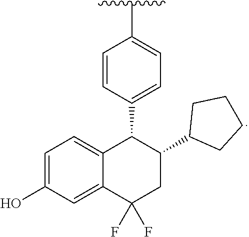 [SMILES: *c1ccc([C@H]2c3ccc(O)cc3C(F)(F)C[C@H]2C2CCCC2)cc1]

In some embodiments, ERBM is

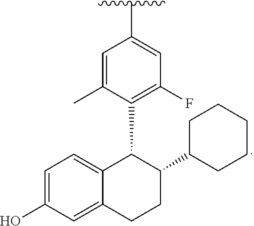 [SMILES: *c1cc(C)c([C@H]2c3ccc(O)cc3CC[C@H]2C2CCCCC2)c(F)c1]

In some embodiments, ERBM is

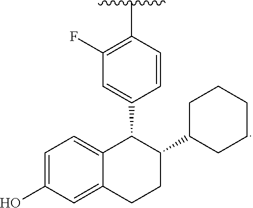 [SMILES: *c1ccc([C@H]2c3ccc(O)cc3CC[C@H]2C2CCCCC2)cc1F]

In some embodiments, ERBM is

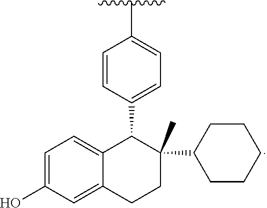 [SMILES: *c1ccc([C@H]2c3ccc(O)cc3CC[C@@]2(C)C2CCCCC2)cc1]

In some embodiments, ERBM is

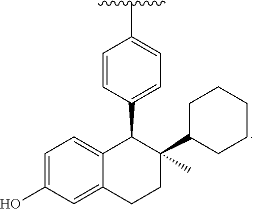 [SMILES: *c1ccc([C@@H]2c3ccc(O)cc3CC[C@]2(C)C2CCCCC2)cc1]

In some embodiments, ERBM is

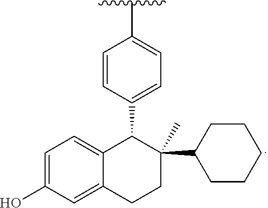 [SMILES: *c1ccc([C@H]2c3ccc(O)cc3CC[C@]2(C)C2CCCCC2)cc1]

In some embodiments, ERBM is

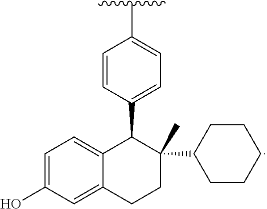 [SMILES: *c1ccc([C@@H]2c3ccc(O)cc3CC[C@@]2(C)C2CCCCC2)cc1]

In some embodiments, ERBM is

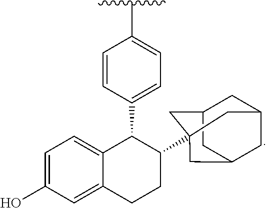 [SMILES: *c1ccc([C@H]2c3ccc(O)cc3CC[C@H]2C23CC4CC(CC(C4)C2)C3)cc1]

In some embodiments, ERBM is

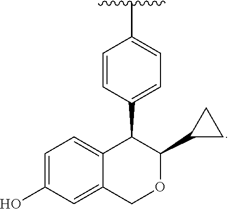 [SMILES: *c1ccc([C@@H]2c3ccc(O)cc3CO[C@@H]2C2CC2)cc1]

In some embodiments, ERBM is

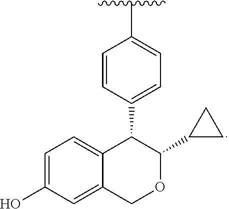 [SMILES: *c1ccc([C@H]2c3ccc(O)cc3CO[C@H]2C2CC2)cc1]

In some embodiments, ERBM is

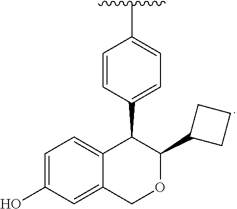 [SMILES: *c1ccc([C@@H]2c3ccc(O)cc3CO[C@@H]2C2CCC2)cc1]

In some embodiments, ERBM is

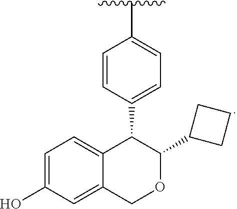 [SMILES: *c1ccc([C@H]2c3ccc(O)cc3CO[C@H]2C2CCC2)cc1]

In some embodiments, ERBM is

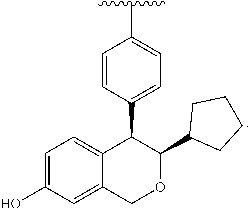 [SMILES: *c1ccc([C@@H]2c3ccc(O)cc3CO[C@@H]2C2CCCC2)cc1]

In some embodiments, ERBM is

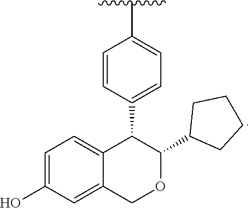 [SMILES: *c1ccc([C@H]2c3ccc(O)cc3CO[C@H]2C2CCCC2)cc1]

In some embodiments, ERBM is

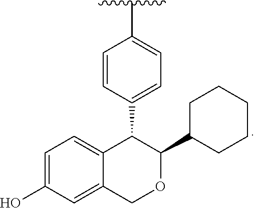 [SMILES: *c1ccc([C@H]2c3ccc(O)cc3CO[C@@H]2C2CCCCC2)cc1]

In some embodiments, ERBM is

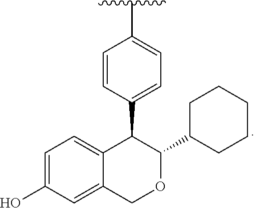 [SMILES: *c1ccc([C@@H]2c3ccc(O)cc3CO[C@H]2C2CCCCC2)cc1]

In some embodiments, ERBM is

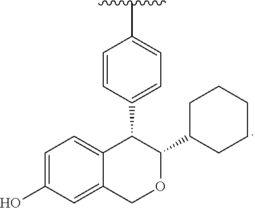 [SMILES: *c1ccc([C@H]2c3ccc(O)cc3CO[C@H]2C2CCCCC2)cc1]

In some embodiments, ERBM is

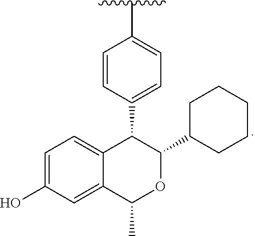 [SMILES: *c1ccc([C@H]2c3ccc(O)cc3[C@@H](C)O[C@H]2C2CCCCC2)cc1]

In some embodiments, ERBM is

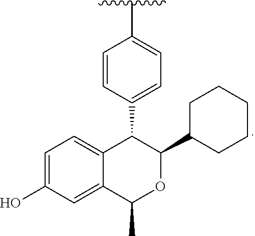 [SMILES: *c1ccc([C@H]2c3ccc(O)cc3[C@H](C)O[C@@H]2C2CCCCC2)cc1]

In some embodiments, ERBM is

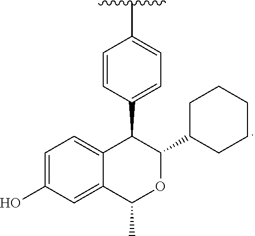 [SMILES: *c1ccc([C@@H]2c3ccc(O)cc3[C@@H](C)O[C@H]2C2CCCCC2)cc1]

In some embodiments, ERBM is

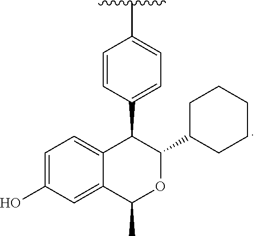 [SMILES: *c1ccc([C@@H]2c3ccc(O)cc3[C@H](C)O[C@H]2C2CCCCC2)cc1]

In some embodiments, ERBM is

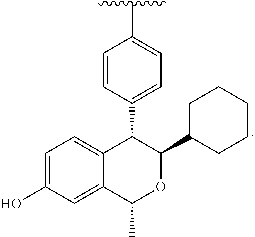 [SMILES: *c1ccc([C@H]2c3ccc(O)cc3[C@@H](C)O[C@@H]2C2CCCCC2)cc1]

In some embodiments, ERBM is

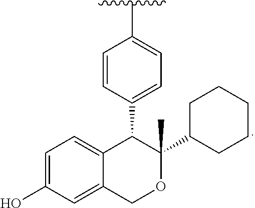 [SMILES: *c1ccc([C@H]2c3ccc(O)cc3CO[C@@]2(C)C2CCCCC2)cc1]

In some embodiments, ERBM is

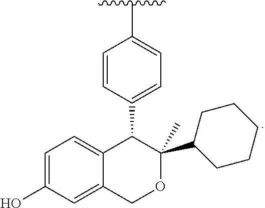 [SMILES: *c1ccc([C@H]2c3ccc(O)cc3CO[C@]2(C)C2CCCCC2)cc1]

In some embodiments, ERBM is

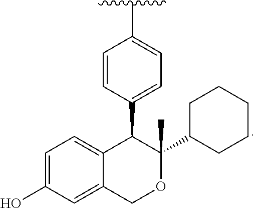 [SMILES: *c1ccc([C@@H]2c3ccc(O)cc3CO[C@@]2(C)C2CCCCC2)cc1]

In some embodiments, ERBM is

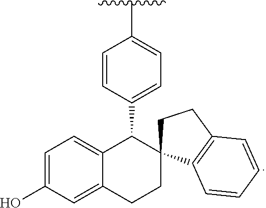 [SMILES: *c1ccc([C@H]2c3ccc(O)cc3CC[C@]23CCc2ccccc23)cc1]

In some embodiments, ERBM is

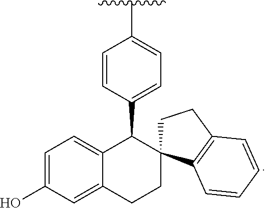 [SMILES: *c1ccc([C@@H]2c3ccc(O)cc3CC[C@@]23CCc2ccccc23)cc1]

In some embodiments, ERBM is

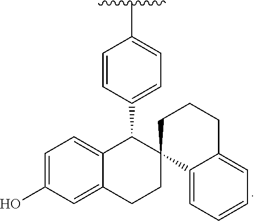 [SMILES: *c1ccc([C@H]2c3ccc(O)cc3CC[C@]23CCCc2ccccc23)cc1]

In some embodiments, ERBM is

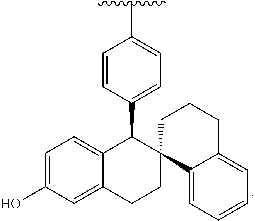 [SMILES: *c1ccc([C@@H]2c3ccc(O)cc3CC[C@@]23CCCc2ccccc23)cc1]

In some embodiments, ERBM is

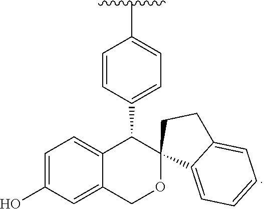 [SMILES: *c1ccc([C@H]2c3ccc(O)cc3CO[C@]23CCc2ccccc23)cc1]

In some embodiments, ERBM is

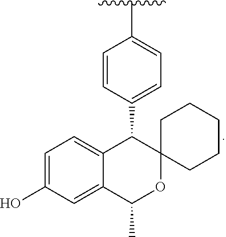 [SMILES: *c1ccc([C@H]2c3ccc(O)cc3[C@@H](C)OC23CCCCC3)cc1]

In some embodiments, ERBM is

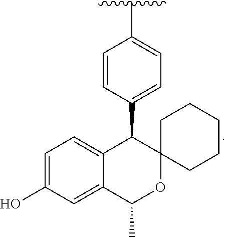 [SMILES: *c1ccc([C@@H]2c3ccc(O)cc3[C@@H](C)OC23CCCCC3)cc1]

In some embodiments, ERBM is

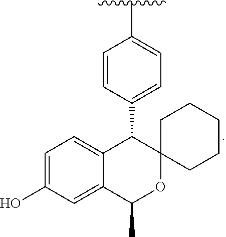 [SMILES: *c1ccc([C@H]2c3ccc(O)cc3[C@H](C)OC23CCCCC3)cc1]

In some embodiments, ERBM is

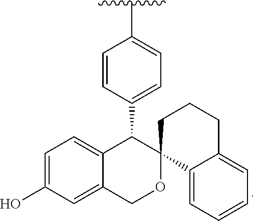 [SMILES: *c1ccc([C@H]2c3ccc(O)cc3CO[C@]23CCCc2ccccc23)cc1]

In some embodiments, ERBM is

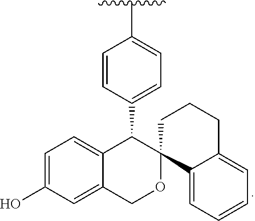 [SMILES: *c1ccc([C@H]2c3ccc(O)cc3CO[C@@]23CCCc2ccccc23)cc1]

In some embodiments, ERBM is

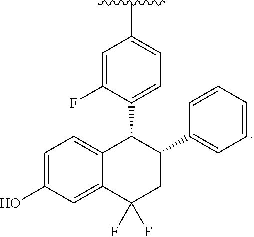 [SMILES: *c1ccc([C@H]2c3ccc(O)cc3C(F)(F)C[C@H]2c2ccccc2)c(F)c1]

In some embodiments, ERBM is

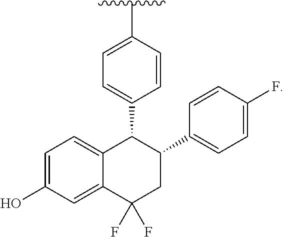 [SMILES: *c1ccc([C@H]2c3ccc(O)cc3C(F)(F)C[C@H]2c2ccc(F)cc2)cc1]

In some embodiments, ERBM is

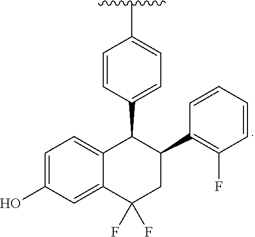 [SMILES: *c1ccc([C@@H]2c3ccc(O)cc3C(F)(F)C[C@@H]2c2ccccc2F)cc1]

In some embodiments, ERBM is

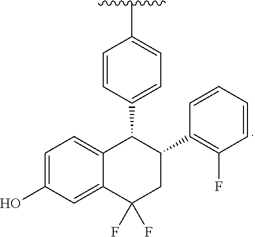 [SMILES: *c1ccc([C@H]2c3ccc(O)cc3C(F)(F)C[C@H]2c2ccccc2F)cc1]

In some embodiments, ERBM is

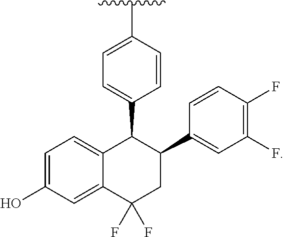 [SMILES: *c1ccc([C@@H]2c3ccc(O)cc3C(F)(F)C[C@@H]2c2ccc(F)c(F)c2)cc1]

In some embodiments, ERBM is

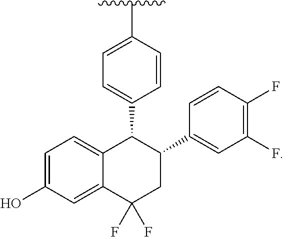 [SMILES: *c1ccc([C@H]2c3ccc(O)cc3C(F)(F)C[C@H]2c2ccc(F)c(F)c2)cc1]

In some embodiments, ERBM is

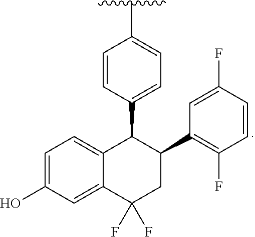 [SMILES: *c1ccc([C@@H]2c3ccc(O)cc3C(F)(F)C[C@@H]2c2cc(F)ccc2F)cc1]

In some embodiments, ERBM is

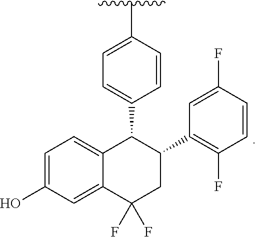 [SMILES: *c1ccc([C@H]2c3ccc(O)cc3C(F)(F)C[C@H]2c2cc(F)ccc2F)cc1]

In some embodiments, ERBM is

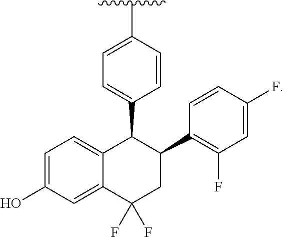 [SMILES: *c1ccc([C@@H]2c3ccc(O)cc3C(F)(F)C[C@@H]2c2ccc(F)cc2F)cc1]

In some embodiments, ERBM is

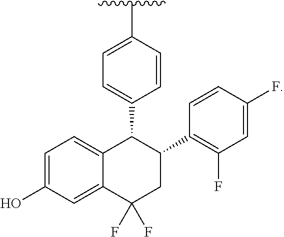 [SMILES: *c1ccc([C@H]2c3ccc(O)cc3C(F)(F)C[C@H]2c2ccc(F)cc2F)cc1]

In some embodiments, ERBM is

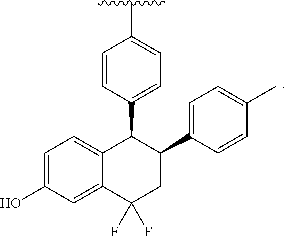 [SMILES: *c1ccc([C@@H]2c3ccc(O)cc3C(F)(F)C[C@@H]2c2ccc(C)cc2)cc1]

In some embodiments, ERBM is

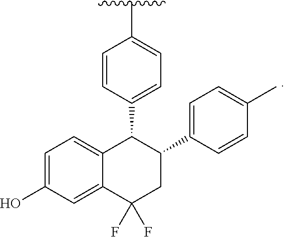 [SMILES: *c1ccc([C@H]2c3ccc(O)cc3C(F)(F)C[C@H]2c2ccc(C)cc2)cc1]

In some embodiments, ERBM is

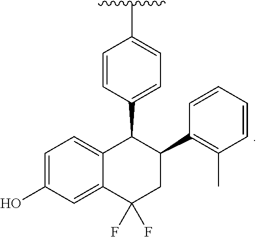 [SMILES: *c1ccc([C@@H]2c3ccc(O)cc3C(F)(F)C[C@@H]2c2ccccc2C)cc1]

In some embodiments, ERBM is

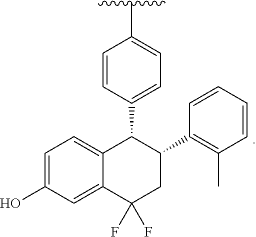 [SMILES: *c1ccc([C@H]2c3ccc(O)cc3C(F)(F)C[C@H]2c2ccccc2C)cc1]

In some embodiments, ERBM is

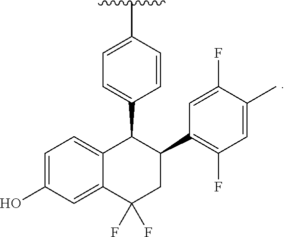 [SMILES: *c1ccc([C@@H]2c3ccc(O)cc3C(F)(F)C[C@@H]2c2cc(F)c(C)cc2F)cc1]

In some embodiments, ERBM is

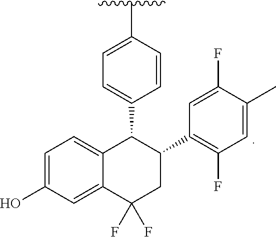 [SMILES: *c1ccc([C@H]2c3ccc(O)cc3C(F)(F)C[C@H]2c2cc(F)c(C)cc2F)cc1]

In some embodiments, ERBM is

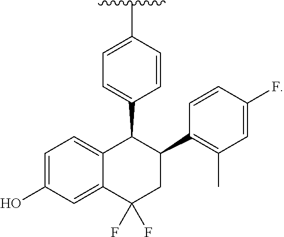 [SMILES: *c1ccc([C@@H]2c3ccc(O)cc3C(F)(F)C[C@@H]2c2ccc(F)cc2C)cc1]

In some embodiments, ERBM is

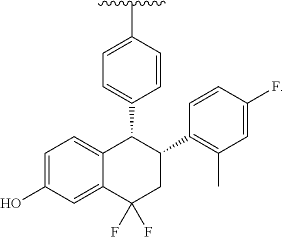 [SMILES: *c1ccc([C@H]2c3ccc(O)cc3C(F)(F)C[C@H]2c2ccc(F)cc2C)cc1]

In some embodiments, ERBM is

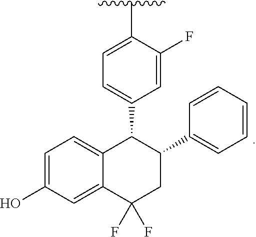 [SMILES: *c1ccc([C@H]2c3ccc(O)cc3C(F)(F)C[C@H]2c2ccccc2)cc1F]

In some embodiments, ERBM is

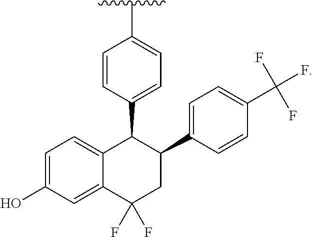 [SMILES: *c1ccc([C@@H]2c3ccc(O)cc3C(F)(F)C[C@@H]2c2ccc(C(F)(F)F)cc2)cc1]

In some embodiments, ERBM is

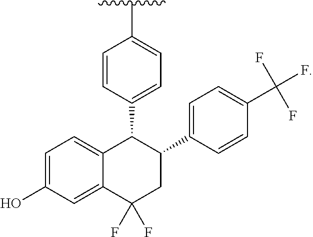 [SMILES: *c1ccc([C@H]2c3ccc(O)cc3C(F)(F)C[C@H]2c2ccc(C(F)(F)F)cc2)cc1]

In some embodiments, ERBM is

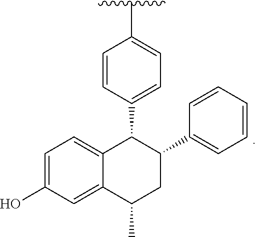 [SMILES: *c1ccc([C@H]2c3ccc(O)cc3[C@@H](C)C[C@H]2c2ccccc2)cc1]

In some embodiments, ERBM is

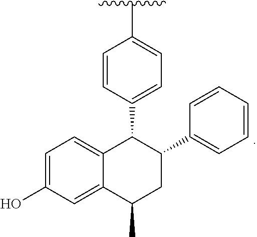 [SMILES: *c1ccc([C@H]2c3ccc(O)cc3[C@H](C)C[C@H]2c2ccccc2)cc1]

In some embodiments, ERBM is

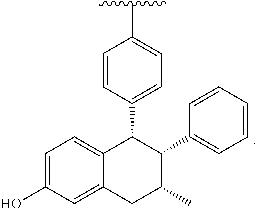 [SMILES: *c1ccc([C@H]2c3ccc(O)cc3C[C@@H](C)[C@H]2c2ccccc2)cc1]

In some embodiments, ERBM is

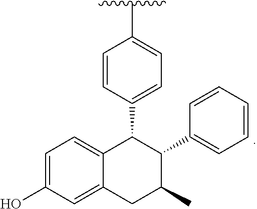 [SMILES: *c1ccc([C@H]2c3ccc(O)cc3C[C@H](C)[C@H]2c2ccccc2)cc1]

In some embodiments, ERBM is

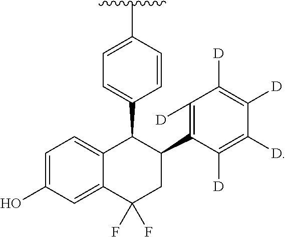

In some embodiments, ERBM is

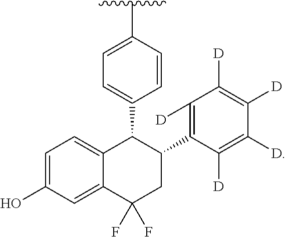

In some embodiments, ERBM is

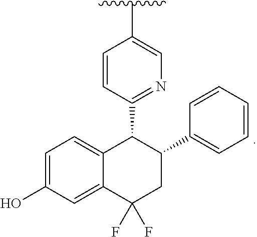 [SMILES: *c1ccc([C@H]2c3ccc(O)cc3C(F)(F)C[C@H]2c2ccccc2)nc1]

In some embodiments, ERBM is

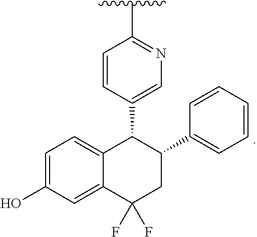 [SMILES: *c1ccc([C@H]2c3ccc(O)cc3C(F)(F)C[C@H]2c2ccccc2)cn1]

In some embodiments, ERBM is

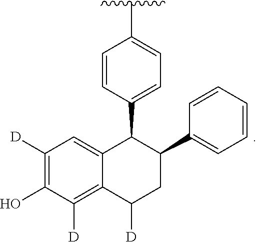 [SMILES: *c1ccc([C@@H]2c3cc([2H])c(O)c([2H])c3C([2H])C[C@@H]2c2ccccc2)cc1]

In some embodiments, ERBM is

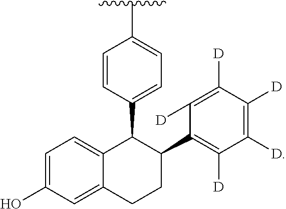

In some embodiments, ERBM is

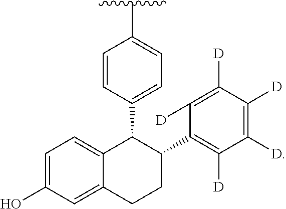

In some embodiments, ERBM is

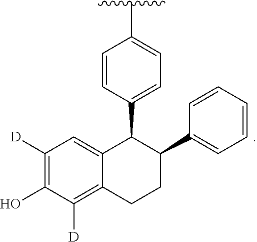 [SMILES: *c1ccc([C@@H]2c3cc([2H])c(O)c([2H])c3CC[C@@H]2c2ccccc2)cc1]

In some embodiments, ERBM is

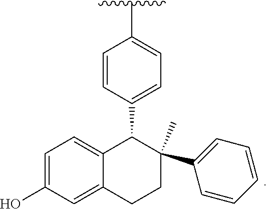 [SMILES: *c1ccc([C@H]2c3ccc(O)cc3CC[C@]2(C)c2ccccc2)cc1]

In some embodiments, ERBM is

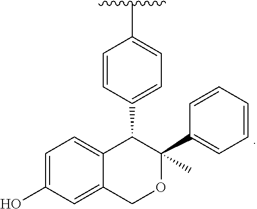 [SMILES: *c1ccc([C@H]2c3ccc(O)cc3CO[C@]2(C)c2ccccc2)cc1]

In some embodiments, ERBM is

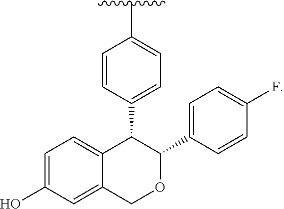 [SMILES: *c1ccc([C@H]2c3ccc(O)cc3CO[C@H]2c2ccc(F)cc2)cc1]

In some embodiments, ERBM is

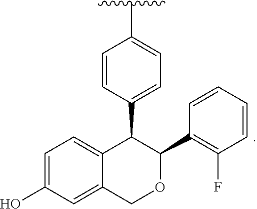 [SMILES: *c1ccc([C@@H]2c3ccc(O)cc3CO[C@@H]2c2ccccc2F)cc1]

In some embodiments, ERBM is

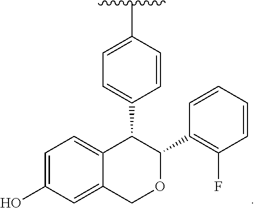 [SMILES: *c1ccc([C@H]2c3ccc(O)cc3CO[C@H]2c2ccccc2F)cc1]

In some embodiments, ERBM is

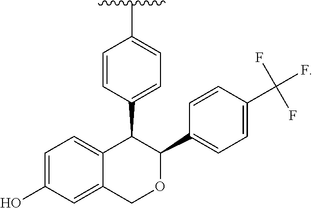 [SMILES: *c1ccc([C@@H]2c3ccc(O)cc3CO[C@@H]2c2ccc(C(F)(F)F)cc2)cc1]

In some embodiments, ERBM is

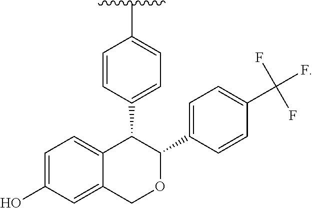 [SMILES: *c1ccc([C@H]2c3ccc(O)cc3CO[C@H]2c2ccc(C(F)(F)F)cc2)cc1]

In some embodiments, ERBM is

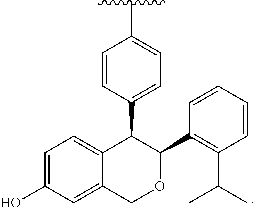 [SMILES: *c1ccc([C@@H]2c3ccc(O)cc3CO[C@@H]2c2ccccc2C(C)C)cc1]

In some embodiments, ERBM is

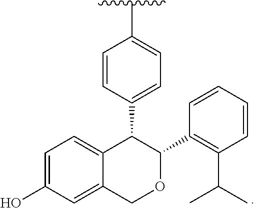 [SMILES: *c1ccc([C@H]2c3ccc(O)cc3CO[C@H]2c2ccccc2C(C)C)cc1]

In some embodiments, ERBM is

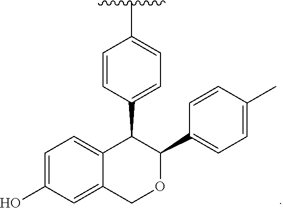 [SMILES: *c1ccc([C@@H]2c3ccc(O)cc3CO[C@@H]2c2ccc(C)cc2)cc1]

In some embodiments, ERBM is

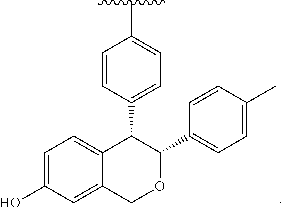 [SMILES: *c1ccc([C@H]2c3ccc(O)cc3CO[C@H]2c2ccc(C)cc2)cc1]

In some embodiments, ERBM is

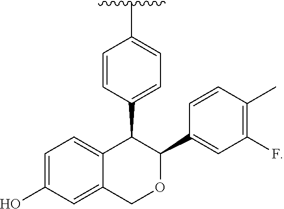 [SMILES: *c1ccc([C@@H]2c3ccc(O)cc3CO[C@@H]2c2ccc(C)c(F)c2)cc1]

In some embodiments, ERBM is

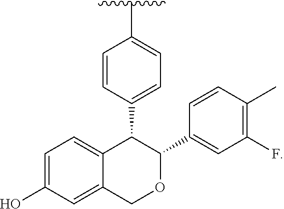 [SMILES: *c1ccc([C@H]2c3ccc(O)cc3CO[C@H]2c2ccc(C)c(F)c2)cc1]

In some embodiments, ERBM is

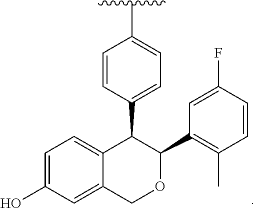 [SMILES: *c1ccc([C@@H]2c3ccc(O)cc3CO[C@@H]2c2cc(F)ccc2C)cc1]

In some embodiments, ERBM is

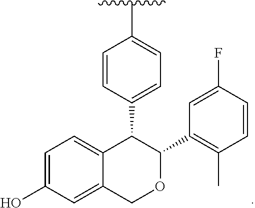 [SMILES: *c1ccc([C@H]2c3ccc(O)cc3CO[C@H]2c2cc(F)ccc2C)cc1]

In some embodiments, ERBM is

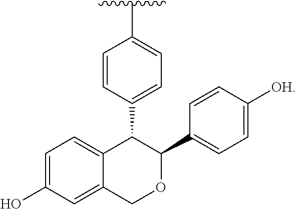 [SMILES: *c1ccc([C@H]2c3ccc(O)cc3CO[C@@H]2c2ccc(O)cc2)cc1]

In some embodiments, ERBM is

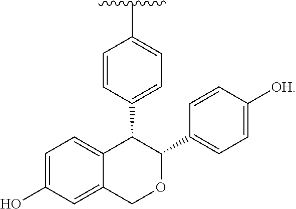 [SMILES: *c1ccc([C@H]2c3ccc(O)cc3CO[C@H]2c2ccc(O)cc2)cc1]

In some embodiments, ERBM is

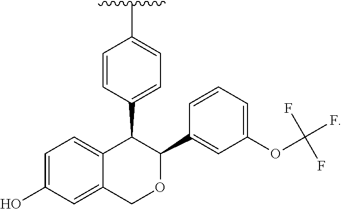 [SMILES: *c1ccc([C@@H]2c3ccc(O)cc3CO[C@@H]2c2cccc(OC(F)(F)F)c2)cc1]

In some embodiments, ERBM is

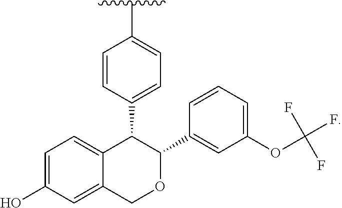 [SMILES: *c1ccc([C@H]2c3ccc(O)cc3CO[C@H]2c2cccc(OC(F)(F)F)c2)cc1]

In some embodiments, ERBM is

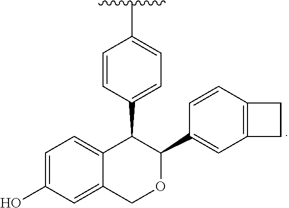 [SMILES: *c1ccc([C@@H]2c3ccc(O)cc3CO[C@@H]2c2ccc3c(c2)CC3)cc1]

In some embodiments, ERBM is

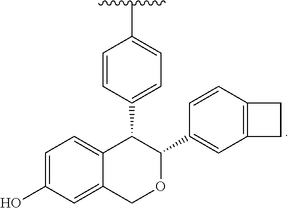 [SMILES: *c1ccc([C@H]2c3ccc(O)cc3CO[C@H]2c2ccc3c(c2)CC3)cc1]

In some embodiments, ERBM is

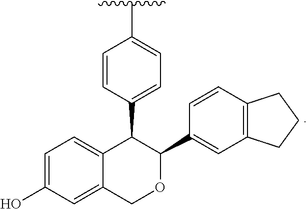 [SMILES: *c1ccc([C@@H]2c3ccc(O)cc3CO[C@@H]2c2ccc3c(c2)CCC3)cc1]

In some embodiments, ERBM is

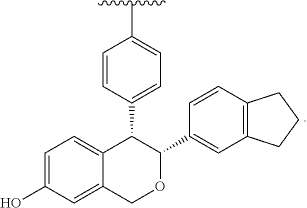 [SMILES: *c1ccc([C@H]2c3ccc(O)cc3CO[C@H]2c2ccc3c(c2)CCC3)cc1]

In some embodiments, ERBM is

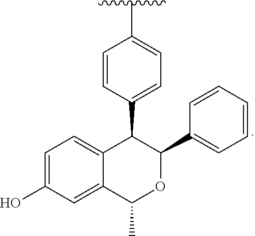 [SMILES: *c1ccc([C@@H]2c3ccc(O)cc3[C@@H](C)O[C@@H]2c2ccccc2)cc1]

In some embodiments, ERBM is

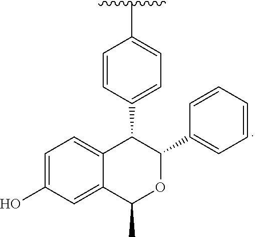 [SMILES: *c1ccc([C@H]2c3ccc(O)cc3[C@H](C)O[C@H]2c2ccccc2)cc1]

In some embodiments, ERBM is

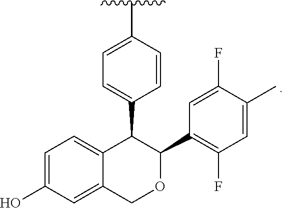 [SMILES: *c1ccc([C@@H]2c3ccc(O)cc3CO[C@@H]2c2cc(F)c(C)cc2F)cc1]

In some embodiments, ERBM is

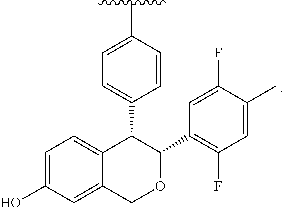 [SMILES: *c1ccc([C@H]2c3ccc(O)cc3CO[C@H]2c2cc(F)c(C)cc2F)cc1]

In some embodiments, ERBM is

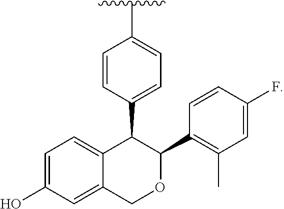 [SMILES: *c1ccc([C@@H]2c3ccc(O)cc3CO[C@@H]2c2ccc(F)cc2C)cc1]

In some embodiments, ERBM is

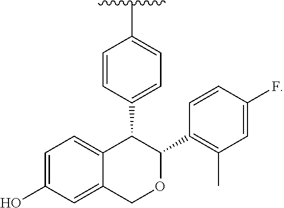 [SMILES: *c1ccc([C@H]2c3ccc(O)cc3CO[C@H]2c2ccc(F)cc2C)cc1]

In some embodiments, ERBM is

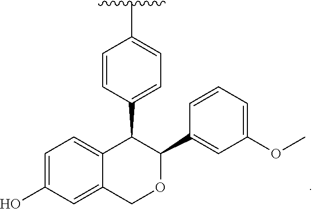 [SMILES: *c1ccc([C@@H]2c3ccc(O)cc3CO[C@@H]2c2cccc(OC)c2)cc1]

In some embodiments, ERBM is

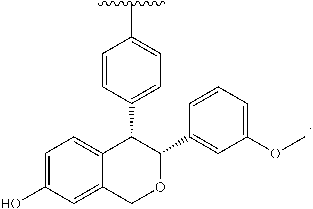 [SMILES: *c1ccc([C@H]2c3ccc(O)cc3CO[C@H]2c2cccc(OC)c2)cc1]

In some embodiments, ERBM is

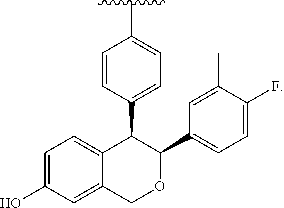 [SMILES: *c1ccc([C@@H]2c3ccc(O)cc3CO[C@@H]2c2ccc(F)c(C)c2)cc1]

In some embodiments, ERBM is

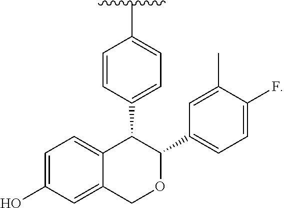 [SMILES: *c1ccc([C@H]2c3ccc(O)cc3CO[C@H]2c2ccc(F)c(C)c2)cc1]

In some embodiments, ERBM is

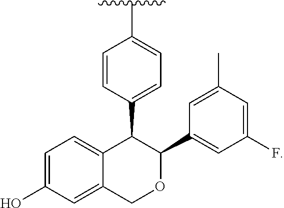 [SMILES: *c1ccc([C@@H]2c3ccc(O)cc3CO[C@@H]2c2cc(C)cc(F)c2)cc1]

In some embodiments, ERBM is

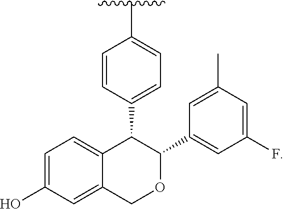 [SMILES: *c1ccc([C@H]2c3ccc(O)cc3CO[C@H]2c2cc(C)cc(F)c2)cc1]

In some embodiments, ERBM is

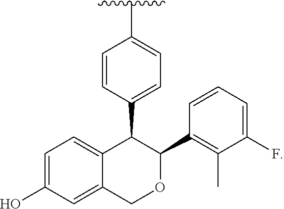 [SMILES: *c1ccc([C@@H]2c3ccc(O)cc3CO[C@@H]2c2cccc(F)c2C)cc1]

In some embodiments, ERBM is

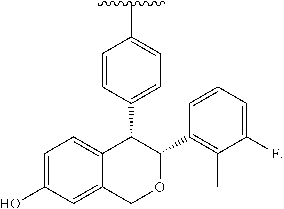 [SMILES: *c1ccc([C@H]2c3ccc(O)cc3CO[C@H]2c2cccc(F)c2C)cc1]

In some embodiments, ERBM is

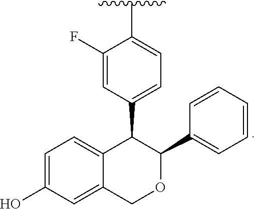 [SMILES: *c1ccc([C@@H]2c3ccc(O)cc3CO[C@@H]2c2ccccc2)cc1F]

In some embodiments, ERBM is

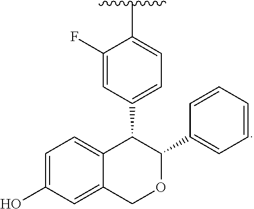 [SMILES: *c1ccc([C@H]2c3ccc(O)cc3CO[C@H]2c2ccccc2)cc1F]

In some embodiments, ERBM is

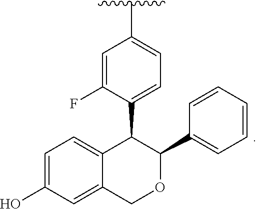 [SMILES: *c1ccc([C@@H]2c3ccc(O)cc3CO[C@@H]2c2ccccc2)c(F)c1]

In some embodiments, ERBM is

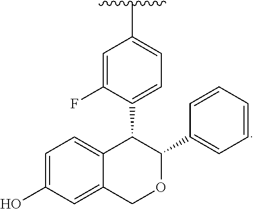 [SMILES: *c1ccc([C@H]2c3ccc(O)cc3CO[C@H]2c2ccccc2)c(F)c1]

In some embodiments, ERBM is

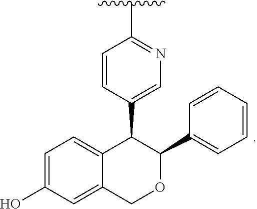 [SMILES: *c1ccc([C@@H]2c3ccc(O)cc3CO[C@@H]2c2ccccc2)cn1]

In some embodiments, ERBM is

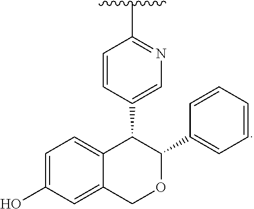 [SMILES: *c1ccc([C@H]2c3ccc(O)cc3CO[C@H]2c2ccccc2)cn1]

In some embodiments, ERBM is

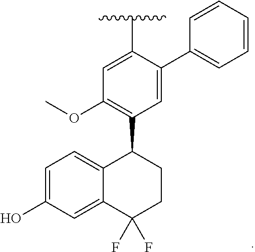 [SMILES: *c1cc(OC)c([C@H]2CCC(F)(F)c3cc(O)ccc32)cc1-c1ccccc1]

In some embodiments, ERBM is

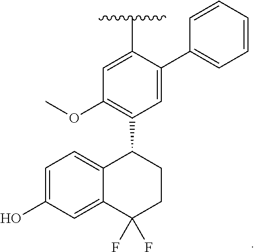 [SMILES: *c1cc(OC)c([C@@H]2CCC(F)(F)c3cc(O)ccc32)cc1-c1ccccc1]

In some embodiments, ERBM is selected from the groups depicted in the compounds in Table 1 or Table 2.

As defined generally above, X¹ is N, NH, CH, CH₂, CH(R^{A1}), C(R^{A1})₂ or C(R^{A1}) as allowed by the other substituents. In some embodiments, X¹ is CH₂.

In some embodiments, X¹ is CH(R^{A1}).

In some embodiments, X¹ is C(R^{A1}).

In some embodiments, is C(R^{A1})₂.

In some embodiments, X¹ is N, CH, or C(R^{A1}).

In some embodiments, X¹ is N. In some embodiments, X¹ is NH. In some embodiments, X¹ is CH. In some embodiments, X¹ is C(R^{A1}).

In some embodiments, X¹ is selected from the groups depicted in the compounds in Table 1 or Table 2.

As defined generally above, X² is N(R^{A2}), O, CH₂, CH(R^{A3}), or C(R^{A3})₂.

In some embodiments, X² is N(R^{A2}). In some embodiments, X² is O. In some embodiments, X² is CH₂. In some embodiments, X² is CH(R^{A3}). In some embodiments, X² is C(R^{A3})₂.

In some embodiments, X² is selected from the groups depicted in the compounds in Table 1 or Table 2.

As generally defined herein, X³ is N(R^{A4}), O, CH₂, CH(R^{A5}), or C(R^{A5})₂.

In some embodiments, X³ is O, CH₂, CH(R^{A5}) or C(R^{A5})₂.

In some embodiments, X³ is O.

In some embodiments, X³ is CH₂.

In some embodiments, X³ is CH(R^{A5}).

In some embodiments, X³ is C(R^{A5})₂.

As defined generally above, each instance of R¹, R₂, R₄, R₅, R⁶, R^{A1}, R^{A2}, R^{A3} R^{A4}, and R^{A5} is independently R^{A} or R^{B}, and is substituted by 0-4 instances of R^{C}.

In some embodiments, R¹ is R^{A} substituted by 0-4 instances of R^{C}. In some embodiments, R¹ is R^{B} substituted by 0-4 instances of R^{C}.

In some embodiments, each R¹ is independently selected from deuterium, C₁₋₆ aliphatic chain substituted with 0-3 instances of halo, halogen, —CN, —SR, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, N(R)S(O)₂NR₂ and —N(R)S(O)₂R, wherein R is H or a C₁₋₆ aliphatic chain substituted with 0-3 instances of halo.

In some embodiments, each R¹ is independently selected from -Me, -Et, —F, —Cl, —SCF₃, —OCF₃, —CF₃, —CN, —OH, —OMe, —NH₂, —NHMe and —NMe₂.

In some embodiments, R¹ is selected from —F and -Me.

In some embodiments, R¹ is —F. In some embodiments, R¹ is -Me.

In some embodiments, R¹ is selected from the groups depicted in the compounds in Table 1 or Table 2.

In some embodiments, R² is R^{A} substituted by 0-4 instances of R^{C}. In some embodiments, R₂ is R^{B} substituted by 0-4 instances of R^{C}.

In some embodiments, each R² is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, each R² is independently selected from -Me, -Et, —F, —Cl, —CF₃, —CN, —OH, —OMe, —NH₂, —NHMe and —NMe₂.

In some embodiments, each R² is independently selected from -Me, —F and —CF₃.

In some embodiments, R² is fluoro. In some embodiments, R² is methyl. In some embodiments, R² is trifluoromethyl.

In some embodiments, R² is selected from the groups depicted in the compounds in Table 1 or Table 2.

In some embodiments, R⁴ is R^{A} substituted by 0-4 instances of R^{C}. In some embodiments, R⁴ is R^{B} substituted by 0-4 instances of R^{C}.

In some embodiments, each R⁴ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, each R⁴ is independently selected from -Me, -Et, —F, —Cl, —CF₃, —CN, —OH, —OMe, —NH₂, —NHMe and —NMe₂.

In some embodiments, R⁴ is —F.

In some embodiments, R⁴ is selected from the groups depicted in the compounds in Table 1 or Table 2.

In some embodiments, R⁵ is R^{A} substituted by 0-4 instances of R^{C}. In some embodiments, R⁵ is R^{B} substituted by 0-4 instances of R^{C}.

In some embodiments, each R⁵ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, each R⁵ is independently selected from -Me, -Et, —F, —Cl, —CF₃, —CN, —OH, —OMe, —NH₂, —NHMe and —NMe₂.

In some embodiments, each R⁵ is independently selected from -Me and —F.

In some embodiments, R⁵ is selected from the groups depicted in the compounds in Table 1 or Table 2.

In some embodiments, each R⁶ is independently selected from -Me, -Et, —F, —Cl, —CF₃, —CO₂H, —CN, —OH, —OMe, —NH₂, —NHMe and —NMe₂

In some embodiments, each R⁶ is independently selected from -Me, and —F.

In some embodiments, Re is selected from the groups depicted in the compounds in Table 1 or Table 2.

In some embodiments, R^{A1} is R^{A} substituted by 0-4 instances of R^{C}. In some embodiments, R^{A1} is R^{B} substituted by 0-4 instances of R^{C}.

In some embodiments, each R^{A1} is independently selected from deuterium, C₁₋₆ aliphatic chain substituted with 0-3 instances of halo or hydroxy, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, each R^{A1} is independently selected from deuterium, a C₁₋₆ aliphatic chain substituted with 0-3 instances of halo or hydroxy and halogen.

In some embodiments, each R^{A1} is selected from a C₁₋₆ aliphatic chain substituted with 0-3 instances of halo or hydroxy and halogen.

In some embodiments, each R^{A1} is a C₁₋₆ aliphatic chain substituted with 0-3 instances of halo or hydroxy.

In some embodiments, each R^{A1} is selected from —F, -Me, -Et, —Pr, —CH═CH—C(CH₃)₃, —CH₂—CH(CH₃)₂, —CH₂—C(CH₃)₂, —CH₂CH₂—C(CH₃)₃, —C(CH₃)₂, —CH₂—CH(CH₃)₂, —C(CH₃)₂OH, —CH₂CH₂—C(CH₃)₂OH, —CH₂CF₃, —CH₂-cyclopropyl, —CH(CH₃)-cyclopropyl, and —CH₂-cyclopentyl.

In some embodiments, each R^{A1} is selected from —F, -Me, -Et, —Pr, —CH═CH—C(CH₃)₃, —CH₂—C(CH₃)₂, —CH₂CH₂—C(CH₃)₃, —C(CH₃)₃, —CH₂—CH(CH₃)₂, —CH₂CH₂—C(CH₃)₂OH and —CH₂-cyclopentyl.

In some embodiments, each R^{A1} is selected from -Me, -Et, —Pr, —CH═CH—C(CH₃)₂, —CH₂—C(CH₃)₃, —CH₂CH₂—C(CH₃)₃, —C(CH₃)₂, —CH₂CH(CH₃)₂, —CH₂CH₂—C(CH₃)₂OH and —CH₂-cyclopentyl.

In some embodiments, each R^{A1} is independently selected from -Me, -Et, —F, —Cl, —CF₃, —CN, —OH, —OMe, —NH₂, —NHMe and —NMe₂.

In some embodiments, each R^{A1} is independently selected from -Me, —F, —OH and —OMe.

In some embodiments, each R^{A1} is independently selected from -Me and —F. In some embodiments, each R^{A1} is independently —F. In some embodiments, each R^{A1} is independently-Me.

In some embodiments, R^{A1} is selected from the groups depicted in the compounds in Table 1 or Table 2.

In some embodiments, R^{A2} is R^{A} substituted by 0-4 instances of R^{C}. In some embodiments, R^{A2} is R^{B} substituted by 0-4 instances of R^{C}.

In some embodiments, R^{A2} is selected from the groups depicted in the compounds in Table 1 or Table 2.

In some embodiments, each R^{A2} is independently selected from deuterium, C₁₋₆ aliphatic chain substituted with 0-3 instances of halo, —S(O)₂R, —S(O)₂NR₂, —S(O)R, —S(O)NR₂, —S(O)(NR)R, —C(O)NR₂, —C(O)OR, and —C(O)R, wherein R is H or a C₁₋₆ aliphatic chain.

In some embodiments, each R^{A2} is independently selected from -Me, -Et, —CH₂CF₃, —S(O)₂Me, —S(O)₂NMe₂, —S(O) Me, —S(O) NMe₂, —C(O) NMe₂, —C(O)NR₂, —C(O) OMe, and —C(O)Me.

In some embodiments, each R^{A2} is independently selected from -Me, —CH₂CF₃, and —C(O)Me. In some embodiments, each R^{A2} is independently selected from -Me and —C(O)Me. In some embodiments, each R^{A1} is independently-C(O)Me. In some embodiments, each R^{A1} is independently-Me.

In some embodiments, R^{A3} is R^{A} substituted by 0-4 instances of R^{C}. In some embodiments, R^{A3} is R^{B} substituted by 0-4 instances of R^{C}.

In some embodiments, each R^{A3} is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, each R^{A3} is independently selected from -Me, -Et, —F, —Cl, —CF₃, —CN, —OH, —OMe, —NH₂, —NHMe and —NMe₂.

In some embodiments, each R^{A3} is independently selected from -Me, —F, —OH and —OMe.

In some embodiments, each R^{A3} is independently selected from -Me and —F. In some embodiments, each R^{A3} is independently —F. In some embodiments, each R^{A3} is independently-Me.

In some embodiments, R^{A3} is selected from the groups depicted in the compounds in Table 1 or Table 2.

As defined generally above, R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur.

In some embodiments, R³ is R^{A} substituted by 0-4 instances of R^{C}. In some embodiments, R³ is R^{B} substituted by 0-4 instances of R^{C}. In some embodiments, two R³ groups are taken together to form a 5-8 membered partially unsaturated or aryl fused ring having 0-2 heteroatoms independently selected from nitrogen, oxygen, and sulfur.

In some embodiments, each R³ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, each R³ is independently selected from -Me, -Et, —F, —Br, —B(OH), —Cl, —CF₃, —CO₂H, —CN, —OH, —OMe, —NH₂, —NHMe and —NMe₂.

In some embodiments, each R³ is independently selected from -Me, —Br, —B(OH), —Cl, —CF₃, —CO₂H, —CN, —OH, —OMe and —NH₂.

In some embodiments, R³ is selected from —F, —OH, and —CO₂H.

some embodiments, R³ is selected from —F and —OH.

In some embodiments, R³ is —OH. In some embodiments, R³ is —CO₂H. In some embodiments, R³ is fluoro. In some embodiments, two R³ groups are taken together to form

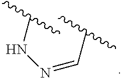 [SMILES: */C=N\N*]

In some embodiments, R³ is selected from -Me, —F, —OH, and —CO₂H. In some embodiments. R³ is selected from -Me, —CF₃ and —F. In some embodiments, R³ is selected from -Me and —F. In some embodiments, R³ is -Me. In some embodiments R³ is —CF₃. In some embodiments, R³ is selected from the groups depicted in the compounds in Table 1 or Table 2.

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, or —B(OR)₂.

In some embodiments, R^{A} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O) (NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, or —B(OR)₂.

In some embodiments, R^{A} is independently selected from deuterium, halogen, —CN, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

In some embodiments, R^{A} is —F. In some embodiments, R^{A} is —OH. In some embodiments, R^{A} is —CO₂H.

In some embodiments, R^{A} is selected from the groups depicted in the compounds in Table 1 or Table 2.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, each R^{B} is C₁₋₆ aliphatic chain substituted with 0-3 instances of halo.

In some embodiments, R^{B} is methyl. In some embodiments, R^{B} together with its R^{C} substituents is trifluoromethyl.

In some embodiments, R^{B} is selected from the groups depicted in the compounds in Table 1 or Table 2.

As defined generally above, each instance of Re is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{C} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR) NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{C} is halo. In some embodiments, R^{C} is —F.

In some embodiments, R^{C} is selected from the groups depicted in the compounds in Table 1 or Table 2.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur.

In some embodiments, R is hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur.

In some embodiments, R is selected from H or a C₁₋₆ aliphatic chain. In some embodiments R is a C₁₋₆ aliphatic chain.

In some embodiments, R is selected from H, -Me, -Et, —Pr, —ⁱPr and -^{t}Bu. In some embodiments, R is selected from H and -Me. In some embodiments, R is -Me. In some embodiments, R is H.

In some embodiments, R is selected from the groups depicted in the compounds in Table 1 or Table 2.

As defined generally above, Ring A and Ring B are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

As defined generally above, Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, Ring A is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, Ring A is phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur.

In some embodiments, Ring A is phenyl; a 5 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur.

In some embodiments, Ring A is a 5-6 membered monocyclic heteroaryl ring having 1-4 heteroatoms independently selected from nitrogen, oxygen, and sulfur (e.g., furanyl, thiophenyl, pyridyl, pyrimidinyl, pyrazinyl, pyridazinyl, pyrrolyl, imidazolyl, pyrazolyl, thiazolyl, oxazolyl, oxadiazolyl, oxathiazolyl).

In some embodiments, Ring A is a 5-membered monocyclic heteroaryl ring having 1-4 heteroatoms independently selected from nitrogen, oxygen, and sulfur (e.g., furanyl, thiophenyl, pyrrolyl, imidazolyl, pyrazolyl, thiazolyl, oxazolyl, oxadiazolyl, oxathiazolyl).

In some embodiments, Ring A is selected from phenyl, furanyl, thiophenyl, pyridyl, pyrimidinyl, pyrazinyl, pyridazinyl, pyrrolyl, imidazolyl, pyrazolyl, thiazolyl, oxazolyl, oxadiazolyl and oxathiazolyl.

In some embodiments, Ring A is selected from phenyl, pyrazolyl, and pyridinyl. In some embodiments, Ring A is selected from phenyl and pyrazolyl.

In some embodiments, Ring A is phenyl. In some embodiments, Ring A is pyrazolyl. In some embodiments, Ring A is pyridinyl.

In some embodiments, Ring A together with its R¹ substituents is selected from

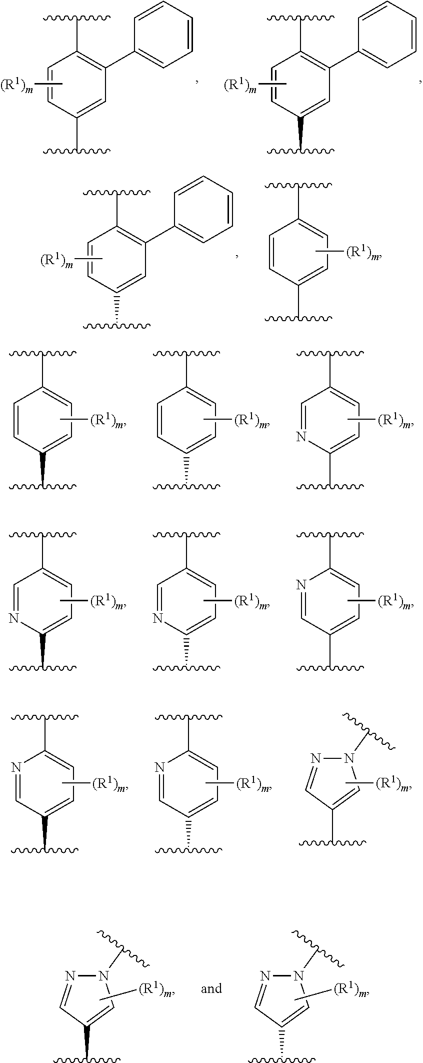

wherein the top attachment point connects to L and the bottom attachment point connects to the six-member ring of the ERBM moiety.

In some embodiments, Ring A together with its R¹ substituents is selected from

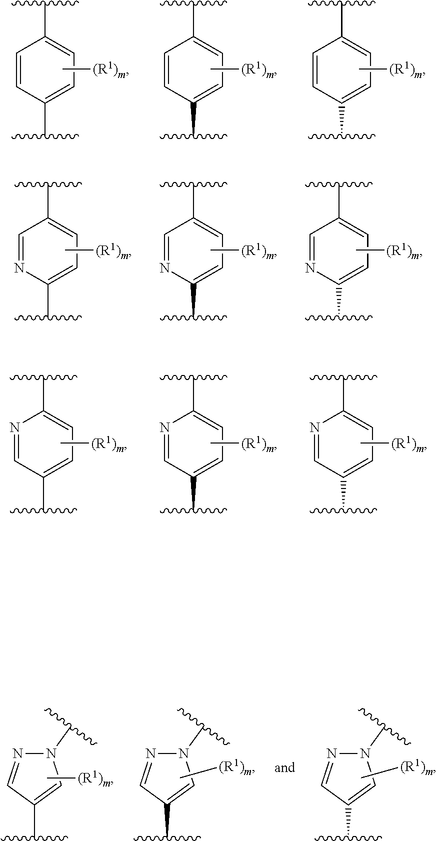

wherein the top attachment point connects to L and the bottom attachment point connects to the six-member ring of the ERBM moiety.

In some embodiments, Ring A together with its R¹ substituents is selected from

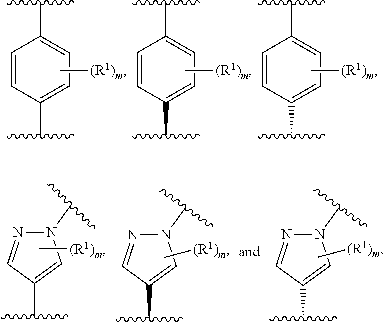

wherein the top attachment point connects to L and the bottom attachment point connects to the six-member ring of the ERBM moiety.

In some embodiments, Ring A together with its R¹ substituents is selected from

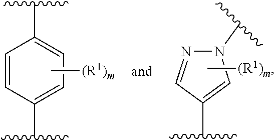

wherein the top attachment point connects to L and the bottom attachment point connects to the six-member ring of the ERBM moiety.

In some embodiments, Ring A together with its R¹ substituents is selected from

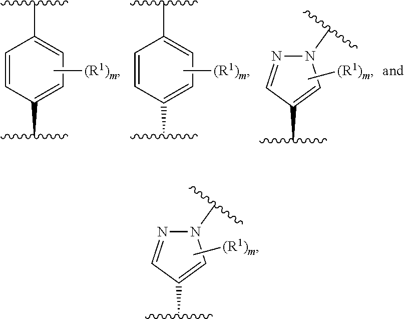

wherein the top attachment point connects to L and the bottom attachment point connects to the six-member ring of the ERBM moiety.

In some embodiments, Ring A together with its R¹ substituents is selected from

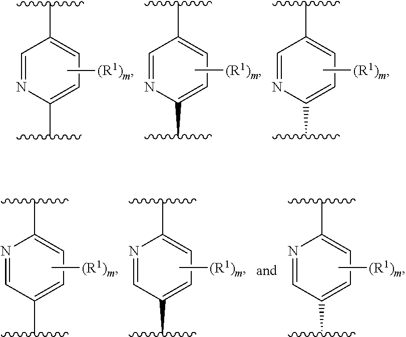

wherein the top attachment point connects to L and the bottom attachment point connects to the six-member ring of the ERBM moiety.

In some embodiments, Ring A together with its R¹ substituents is

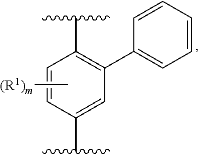

wherein the top attachment point connects to L and the bottom attachment point connects to the six-member ring of the ERBM moiety. In some embodiments, Ring A together with its R¹ substituents is

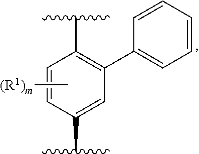

wherein the top attachment point connects to L and the bottom attachment point connects to the six-member ring of the ERBM moiety. In some embodiments, Ring A together with its R¹ substituents is

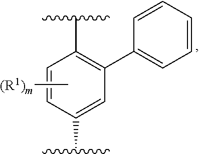

wherein the top attachment point connects to L and the bottom attachment point connects to the six-member ring of the ERBM moiety. In some embodiments, Ring A together with its R¹ substituents is

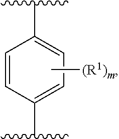

wherein the top attachment point connects to L and the bottom attachment point connects to the six-member ring of the ERBM moiety. In some embodiments, Ring A together with its R¹ substituents is

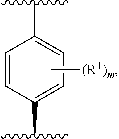

wherein the top attachment point connects to L and the bottom attachment point connects to the six-member ring of the ERBM moiety. In some embodiments, Ring A together with its R¹ substituents is

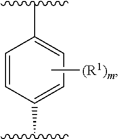

wherein the top attachment point connects to L and the bottom attachment point connects to the six-member ring of the ERBM moiety. In some embodiments, Ring A together with its R¹ substituents is

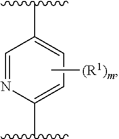

wherein the top attachment point connects to L and the bottom attachment point connects to the six-member ring of the ERBM moiety. In some embodiments, Ring A together with its R¹ substituents is

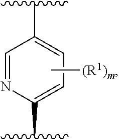

wherein the top attachment point connects to L and the bottom attachment point connects to the six-member ring of the ERBM moiety. In some embodiments, Ring A together with its R¹ substituents is

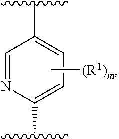

wherein the top attachment point connects to L and the bottom attachment point connects to the six-member ring of the ERBM moiety. In some embodiments, Ring A together with its R¹ substituents is

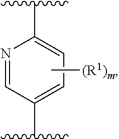

wherein the top attachment point connects to L and the bottom attachment point connects to the six-member ring of the ERBM moiety. In some embodiments, Ring A together with its R¹ substituents is

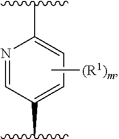

wherein the top attachment point connects to L and the bottom attachment point connects to the six-member ring of the ERBM moiety. In some embodiments, Ring A together with its R¹ substituents is

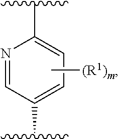

wherein the top attachment point connects to L and the bottom attachment point connects to the six-member ring of the ERBM moiety. In some embodiments, Ring A together with its R¹ substituents is

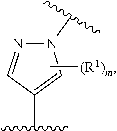

wherein the top attachment point connects to L and the bottom attachment point connects to the six-member ring of the ERBM moiety. In some embodiments, Ring A together with its R¹ substituents is

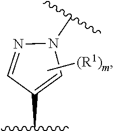

wherein the top attachment point connects to L and the bottom attachment point connects to the six-member ring of the ERBM moiety. In some embodiments, Ring A together with its R¹ substituents is

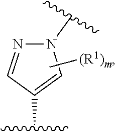

wherein the top attachment point connects to L and the bottom attachment point connects to the six-member ring of the ERBM moiety.

In some embodiments, Ring A is selected from the groups depicted in the compounds in Table 1 or Table 2.

In some embodiments, Ring B is phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, Ring B is phenyl; tetrahydronaphthalenyl; dihydroindenyl; benzocyclobutene; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, Ring B is phenyl; tetrahydronaphthalenyl; dihydroindenyl; benzocyclobutenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring or a 3-7 membered saturated or partially unsaturated monocyclic heterocyclic ring having 1-2 heteroatoms independently selected from nitrogen, oxygen, and sulfur.

In some embodiments, Ring B is phenyl; tetrahydronaphthalenyl; dihydroindenyl; benzocyclobutenyl; a 3-7 membered saturated or partially unsaturated monocyclic heterocyclic ring having 1-2 heteroatoms independently selected from nitrogen, oxygen, and sulfur; a 3-7 membered saturated or partially unsaturated monocyclic carbocyclic ring; or a 5-12 membered saturated or partially unsaturated bicyclic carbocyclic ring.

In some embodiments, Ring B is phenyl; tetrahydronaphthalenyl; dihydroindenyl; benzocyclobutenyl; a 5 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur.

In some embodiments, Ring B is tetrahydronaphthalenyl; dihydroindenyl; or benzocyclobutenyl. In some embodiments, Ring B is tetrahydronaphthalenyl. In some embodiments, Ring B is dihydroindenyl. In some embodiments, Ring B is benzocyclobutenyl.

In some embodiments, Ring B is a 5-6 membered monocyclic heteroaryl ring having 1-4 heteroatoms independently selected from nitrogen, oxygen, and sulfur (e.g., furanyl, thiophenyl, pyridyl, pyrimidinyl, pyrazinyl, pyridazinyl, pyrrolyl, imidazolyl, pyrazolyl, thiazolyl, oxazolyl, oxadiazolyl, oxathiazolyl).

In some embodiments, Ring B is a an 8-10 membered bicyclic heteroaryl ring having 1-4 heteroatoms independently selected from nitrogen, oxygen, and sulfur (e.g., quinolinyl, isoquinolinyl, benzofuranyl, indolyl, benzimidazolyl, benzothiazolyl, benzothiophenyl, dihydrobenzofuranyl, dihydroisobenzofuranyl, indolinyl, isoindolinyl, tetrahydroquinolinyl, tetrahydroisoquinolinyl).

In some embodiments, Ring B is a 3-7 membered saturated or partially unsaturated monocyclic carbocyclic ring (e.g., cyclopropyl, cyclobutyl, cyclopentyl, cyclopentenyl, cyclohexyl, cyclohexenyl, cycloheptyl, cycloheptenyl).

In some embodiments, Ring B is 5-12 membered saturated or partially unsaturated bicyclic carbocyclic ring (e.g., spiro [3.3] heptanyl, spiro [3,4] octanyl, adamantyl, dihydroindenyl (e.g., 2,3-dihydro-1H-indenyl), tetrahydronaphthalenyl, benzocyclobutenyl, bicyclo[1.1.1]pentanyl, bicyclo[2.2.0]hexanyl, bicyclo[2.2.1]hept-2-enyl, bicyclo[3.1.1]heptanyl, bicyclo[4.1.0]heptanyl, bicyclo[3.2.0]heptanyl, bicyclo[2.2.1]heptanyl, bicyclo[2.2.2]oct-2-enyl, bicyclo[2.2.2]octanyl, bicyclo[3.3.1]nonanyl).

In some embodiments, Ring B is a 3-7 membered saturated or partially unsaturated monocyclic heterocyclic ring having 1-2 heteroatoms independently selected from nitrogen, oxygen, and sulfur (e.g., oxetanyl, tetrahydrofuranyl, tetrahydropyranyl, azetidinyl, pyrrolidinyl, piperidinyl, piperazinyl, morpholinyl).

In some embodiments, Ring B is selected from phenyl, cyclopropyl, cyclobutyl, cyclopentyl, cyclopentenyl, cyclohexyl, cyclohexenyl, cycloheptyl, cycloheptenyl, bicyclo[1.1.1]pentanyl, bicyclo[2.2.0]hexanyl, bicyclo[2.2.1]hept-2-enyl, bicyclo[3.1.1]heptanyl, bicyclo[4.1.0]heptanyl, bicyclo[3.2.0]heptanyl, bicyclo[2.2.1]heptanyl, bicyclo[2.2.2]oct-2-enyl, bicyclo[2.2.2]octanyl, bicyclo[3.3.1]nonanyl, spiro[3.3]heptanyl, spiro[3,4] octanyl, adamantyl, dihydroindenyl (e.g., 2,3-dihydro-1H-indenyl), tetrahydronaphthalenyl, benzocyclobutenyl, oxetanyl, tetrahydrofuranyl, tetrahydropyranyl, azetidinyl, pyrrolidinyl, piperidinyl, piperazinyl, morpholinyl, quinolinyl, isoquinolinyl, benzofuranyl, indolyl, benzimidazolyl, benzothiazolyl, benzothiophenyl, dihydrobenzofuranyl, dihydroisobenzofuranyl, indolinyl, isoindolinyl, tetrahydroquinolinyl, tetrahydroisoquinolinyl, furanyl, thiophenyl, pyridyl, pyrimidinyl, pyrazinyl, pyridazinyl, pyrrolyl, imidazolyl, pyrazolyl, thiazolyl, oxazolyl, oxadiazolyl and oxathiazolyl.

In some embodiments, Ring B is selected from phenyl, cyclopentyl, cyclopentenyl, cyclohexyl, cyclohexenyl, bicyclo[1.1.1]pentanyl, bicyclo[2.2.0]hexanyl, bicyclo[2.2.1]hept-2-enyl, bicyclo[3.1.1]heptanyl, bicyclo[4.1.0]heptanyl, bicyclo[3.2.0]heptanyl, bicyclo[2.2.1]heptanyl, bicyclo[2.2.2]oct-2-enyl, bicyclo[2.2.2]octanyl, bicyclo[3.3.1]nonanyl, adamantyl, tetrahydropyranyl, dihydroindenyl (e.g., 2,3-dihydro-1H-indenyl), tetrahydronaphthalenyl, benzocyclobutenyl, dihydrobenzofuranyl, dihydroisobenzofuranyl, indolinyl, isoindolinyl, thiophenyl, pyridyl, pyrimidinyl, pyrazinyl, pyridazinyl and pyrazolyl.

In some embodiments, Ring B is selected from phenyl, cyclopropyl, cyclohexyl, cyclohexenyl, adamantyl, bicyclo[1.1.1]pentanyl, bicyclo[2.2.0]hexanyl, bicyclo[2.2.1]hept-2-enyl, bicyclo[3.1.1]heptanyl, bicyclo[4.1.0]heptanyl, bicyclo[3.2.0]heptanyl, bicyclo[2.2.1]heptanyl, bicyclo[2.2.2]oct-2-enyl, bicyclo[2.2.2]octanyl, bicyclo[3.3.1]nonanyl, tetrahydropyranyl, 2,3-dihydro-1H-indenyl, tetrahydronaphthalenyl, benzocyclobutenyl, pyridinyl, thiophenyl and pyrazolyl.

In some embodiments, Ring B is selected from phenyl, cyclohexyl, cyclohexenyl, adamantyl, tetrahydropyranyl, 2,3-dihydro-1H-indenyl, and pyrazolyl.

In some embodiments, Ring B is phenyl. In some embodiments, Ring B is pyrazolyl. In some embodiments, Ring B is adamantyl. In some embodiments, Ring B is 2,3-dihydro-1H-indenyl. In some embodiments, Ring B is tetrahydrofuranyl.

In some embodiments, Ring B together with its R² substituents is selected from:

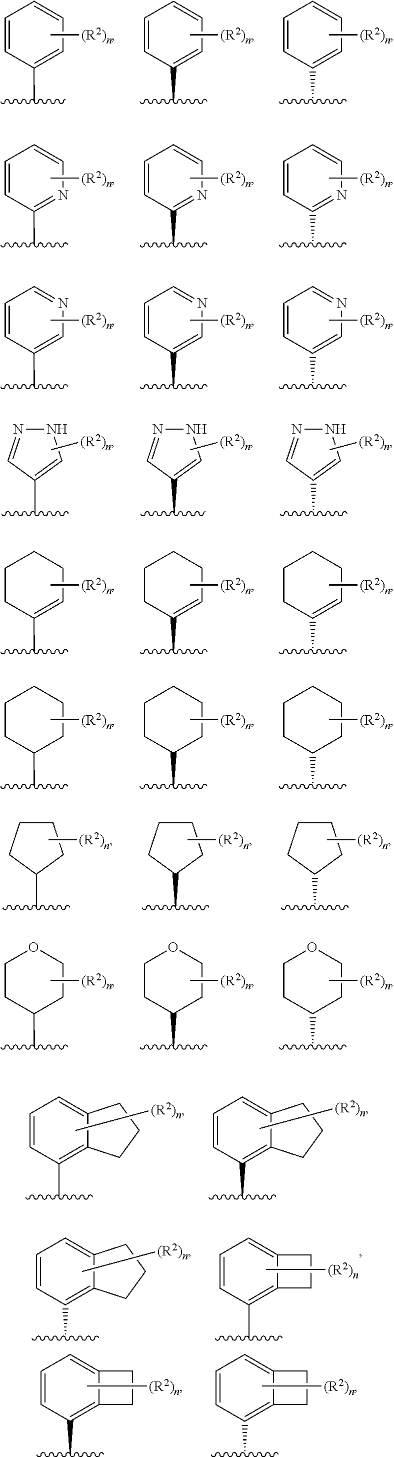

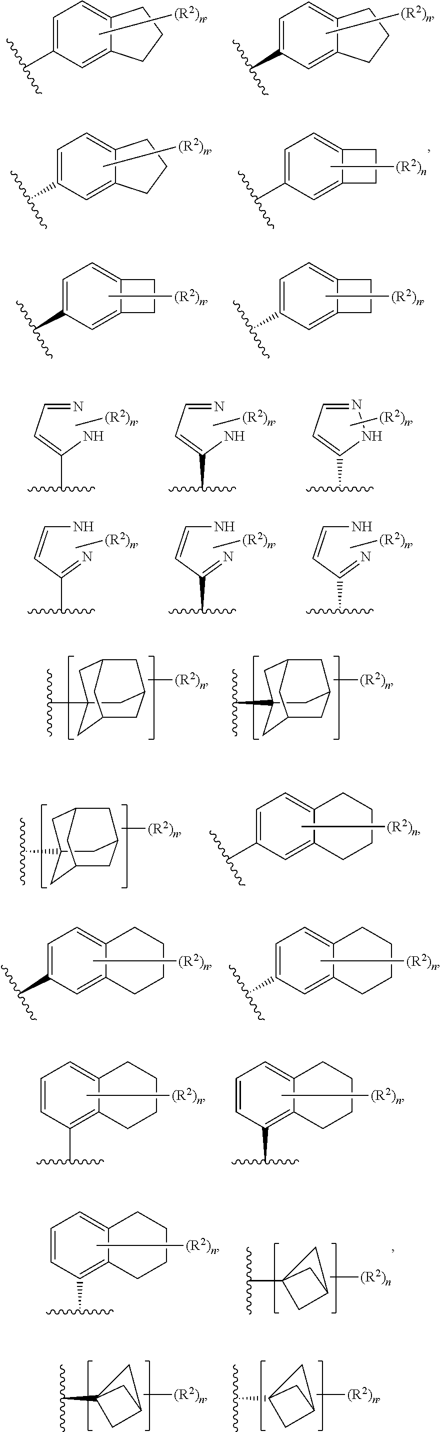

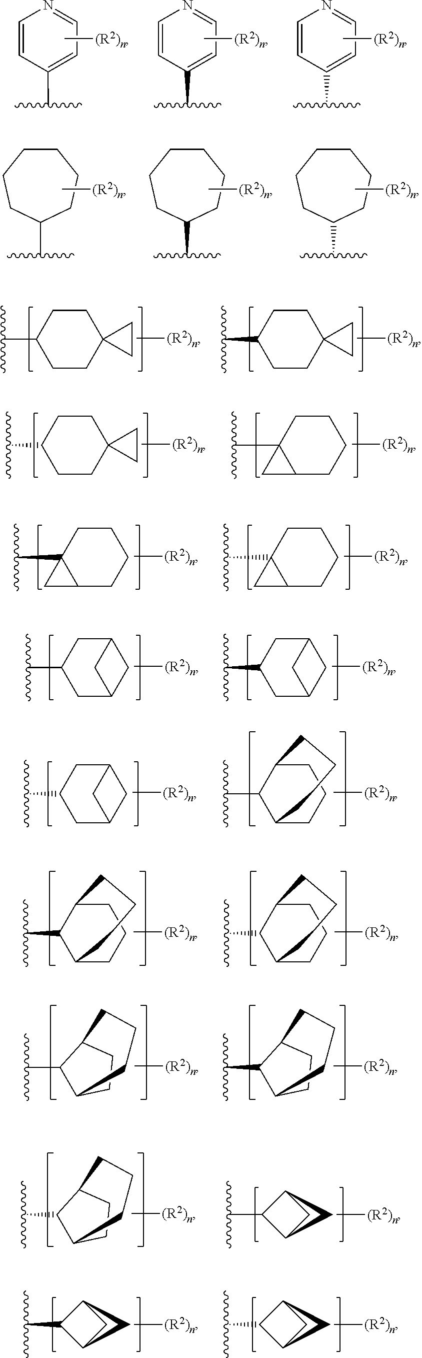

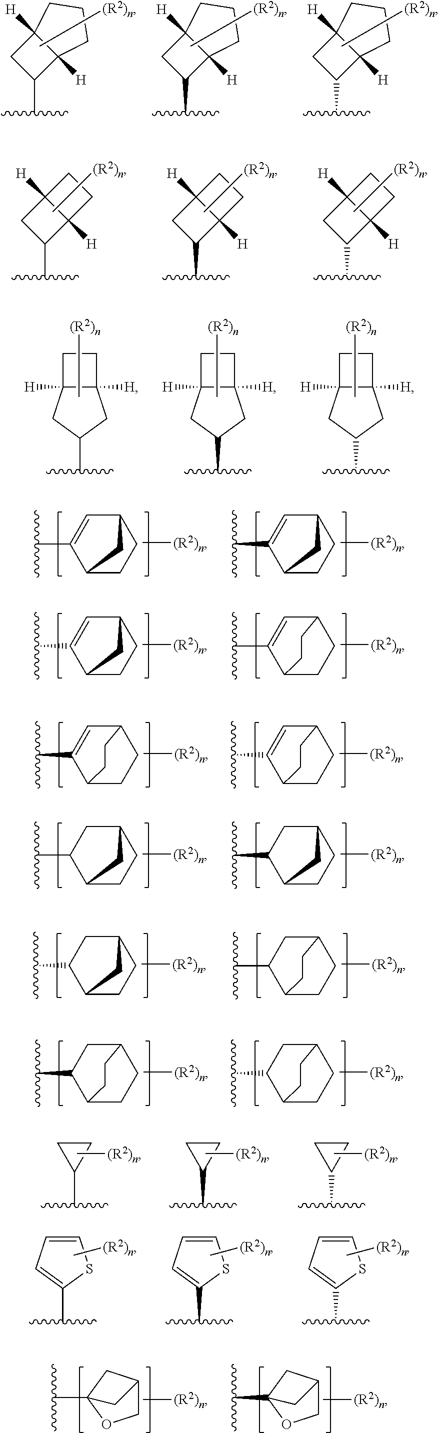

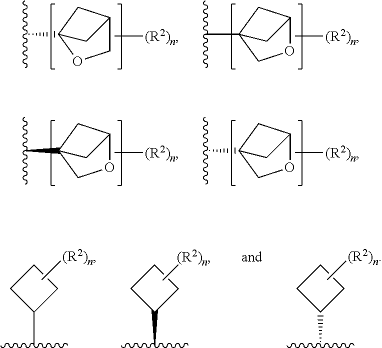

In some embodiments, Ring B together with its R² substituents is

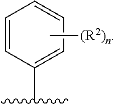

In some embodiments, Ring B together with its R² substituents is

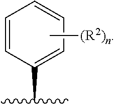

In some embodiments, Ring B together with its R² substituents is

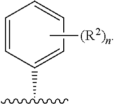

In some embodiments, Ring B together with its R² substituents is

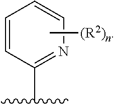

In some embodiments, Ring B together with its R² substituents is

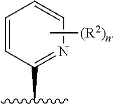

In some embodiments, Ring B together with its R² substituents is

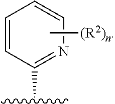

In some embodiments, Ring B together with its R² substituents is

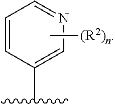

In some embodiments, Ring B together with its R² substituents is

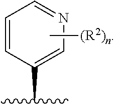

In some embodiments, Ring B together with its R² substituents is

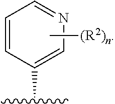

In some embodiments, Ring B together with its R² substituents is

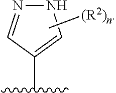

In some embodiments, Ring B together with its R² substituents is

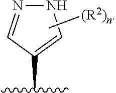

In some embodiments, Ring B together with its R² substituents is

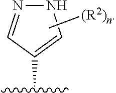

In some embodiments, Ring B together with its R² substituents is

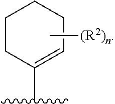

In some embodiments, Ring B together with its R² substituents is

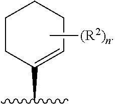

In some embodiments, Ring B together with its R² substituents is

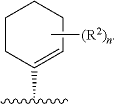

In some embodiments, Ring B together with its R² substituents is

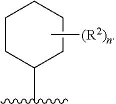

In some embodiments, Ring B together with its R² substituents is

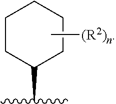

In some embodiments, Ring B together with its R² substituents is

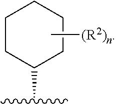

In some embodiments, Ring B together with its R² substituents is

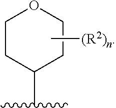

In some embodiments, Ring B together with its R² substituents is

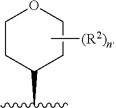

In some embodiments, Ring B together with its R² substituents is

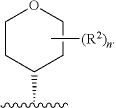

In some embodiments, Ring B together with its R² substituents is

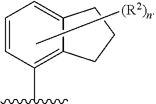

In some embodiments, Ring B together with its R² substituents is

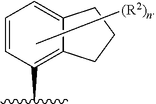

In some embodiments, Ring B together with its R² substituents is

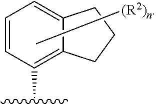

In some embodiments, Ring B together with its R² substituents is

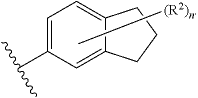

In some embodiments, Ring B together with its R² substituents is

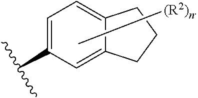

In some embodiments, Ring B together with its R² substituents is

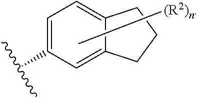

In some embodiments, Ring B together with its R² substituents is

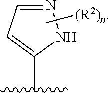

In some embodiments, Ring B together with its R² substituents is

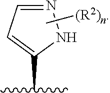

In some embodiments, Ring B together with its R² substituents is

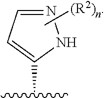

In some embodiments, Ring B together with its R² substituents is

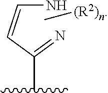

In some embodiments, Ring B together with its R² substituents is

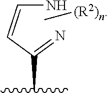

In some embodiments, Ring B together with its R² substituents is

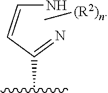

In some embodiments, Ring B together with its R² substituents is

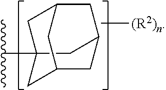

In some embodiments, Ring B together with its R² substituents is

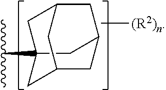

In some embodiments, Ring B together with its R² substituents is

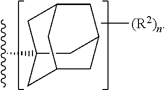

In some embodiments, Ring B together with its R² substituents is

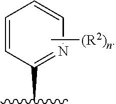

In some embodiments, Ring B together with its R² substituents is

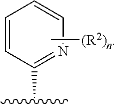

In some embodiments, Ring B together with its R² substituents is

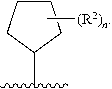

In some embodiments, Ring B together with its R² substituents is

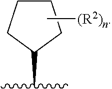

In some embodiments, Ring B together with its R² substituents is

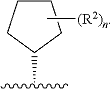

In some embodiments, Ring B together with its R² substituents is

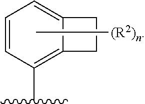

In some embodiments, Ring B together with its R² substituents is

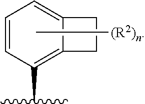

In some embodiments, Ring B together with its R² substituents is

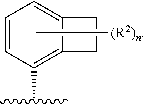

In some embodiments, Ring B together with its R² substituents is

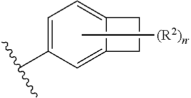

In some embodiments, Ring B together with its R² substituents is

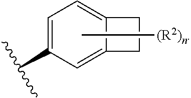

In some embodiments, Ring B together with its R² substituents is

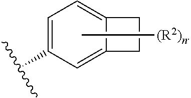

In some embodiments, Ring B together with its R² substituents is

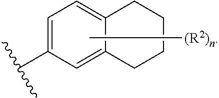

In some embodiments, Ring B together with its R² substituents is

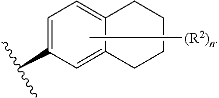

In some embodiments, Ring B together with its R² substituents is

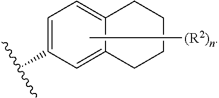

In some embodiments, Ring B together with its R² substituents is

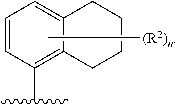

In some embodiments, Ring B together with its R² substituents is

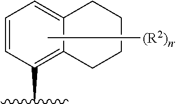

In some embodiments, Ring B together with its R² substituents is

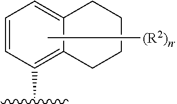

In some embodiments, Ring B together with its R² substituents is

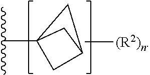

In some embodiments, Ring B together with its R² substituents is

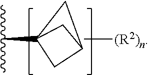

In some embodiments, Ring B together with its R² substituents is

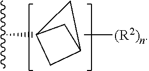

In some embodiments, Ring B together with its R² substituents is

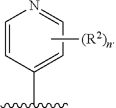

In some embodiments, Ring B together with its R² substituents is

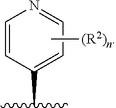

In some embodiments, Ring B together with its R² substituents is

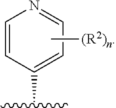

In some embodiments, Ring B together with its R² substituents is

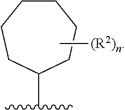

In some embodiments, Ring B together with its R² substituents is

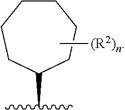

In some embodiments, Ring B together with its R² substituents is

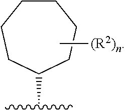

In some embodiments, Ring B together with its R² substituents

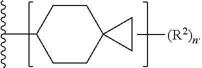

In some embodiments, Ring B together with its R² substituents is

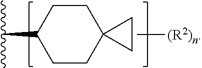

In some embodiments, Ring B together with its R² substituents is

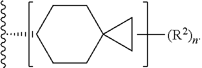

In some embodiments, Ring B together with its R² substituents is

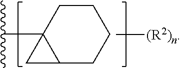

In some embodiments, Ring B together with its R² substituents is

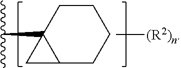

In some embodiments, Ring B together with its R² substituents is

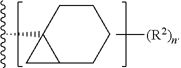

In some embodiments, Ring B together with its R² substituents is

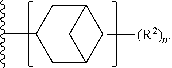

In some embodiments, Ring B together with its R² substituents is

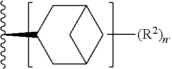

In some embodiments, Ring B together with its R² substituents is

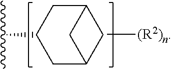

In some embodiments, Ring B together with its R² substituents is

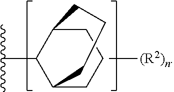

In some embodiments, Ring B together with its R² substituents is

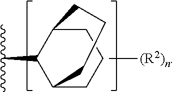

In some embodiments, Ring B together with its R² substituents is

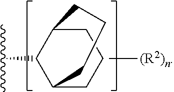

In some embodiments, Ring B together with its R² substituents is

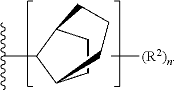

In some embodiments, Ring B together with its R² substituents is

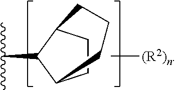

In some embodiments, Ring B together with its R² substituents is

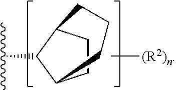

In some embodiments, Ring B together with its R² substituents is

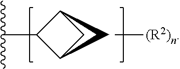

In some embodiments, Ring B together with its R² substituents is

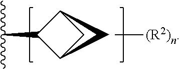

In some embodiments, Ring B together with its R² substituents is

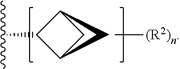

In some embodiments, Ring B together with its R² substituents is

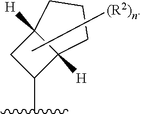

In some embodiments, Ring B together with its R² substituents is

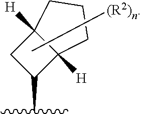

In some embodiments, Ring B together with its R² substituents is

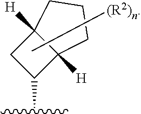

In some embodiments, Ring B together with its R² substituents is

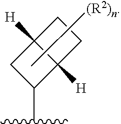

In some embodiments, Ring B together with its R² substituents is

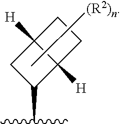

In some embodiments, Ring B together with its R² substituents is

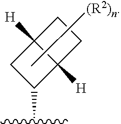

In some embodiments, Ring B together with its R² substituents is

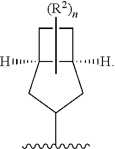

In some embodiments, Ring B together with its R² substituents is

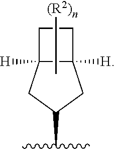

In some embodiments, Ring B together with its R² substituents is

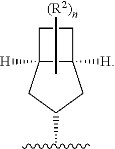

In some embodiments, Ring B together with its R² substituents is

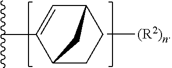

In some embodiments, Ring B together with its R² substituents is

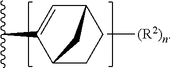

In some embodiments, Ring B together with its R² substituents is

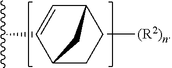

In some embodiments, Ring B together with its R² substituents is

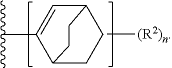

In some embodiments, Ring B together with its R² substituents is

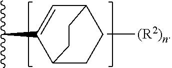

In some embodiments, Ring B together with its R² substituents is

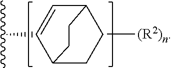

In some embodiments, Ring B together with its R² substituents is

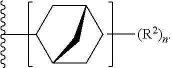

In some embodiments, Ring B together with its R² substituents is

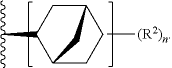

In some embodiments, Ring B together with its R² substituents is

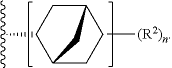

In some embodiments, Ring B together with its R² substituents is

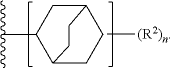

In some embodiments, Ring B together with its R² substituents is

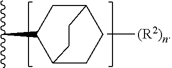

In some embodiments, Ring B together with its R² substituents is

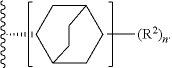

In some embodiments, Ring B together with its R² substituents is

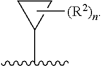

In some embodiments, Ring B together with its R₂ is

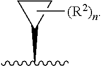

In some embodiments, Ring B together with its R² substituents is

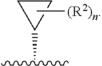

In some embodiments, Ring B together with its R² substituents is

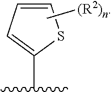

In some embodiments, Ring B together with its R² substituents is

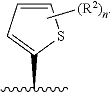

In some embodiments, Ring B together with its R² substituents is

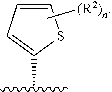

In some embodiments, Ring B together with its R² substituents is

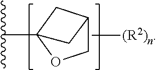

In some embodiments, Ring B together with its R² substituents is

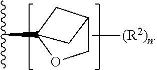

In some embodiments, Ring B together with its R² substituents is

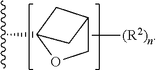

In some embodiments, Ring B together with its R² substituents is

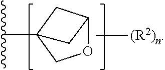

In some embodiments, Ring B together with its R² substituents is

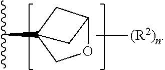

In some embodiments, Ring B together with its R² substituents is

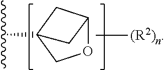

In some embodiments, Ring B together with its R² substituents is

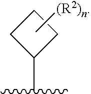

In some embodiments, Ring B together with its R² substituents is

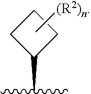

In some embodiments, Ring B together with its R² substituents is

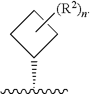

In some embodiments, Ring B is selected from the groups depicted in the compounds in Table 1 or Table 2.

As generally defined herein, Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;

In some embodiments, Ring C together with its R⁶ substituents is selected from:

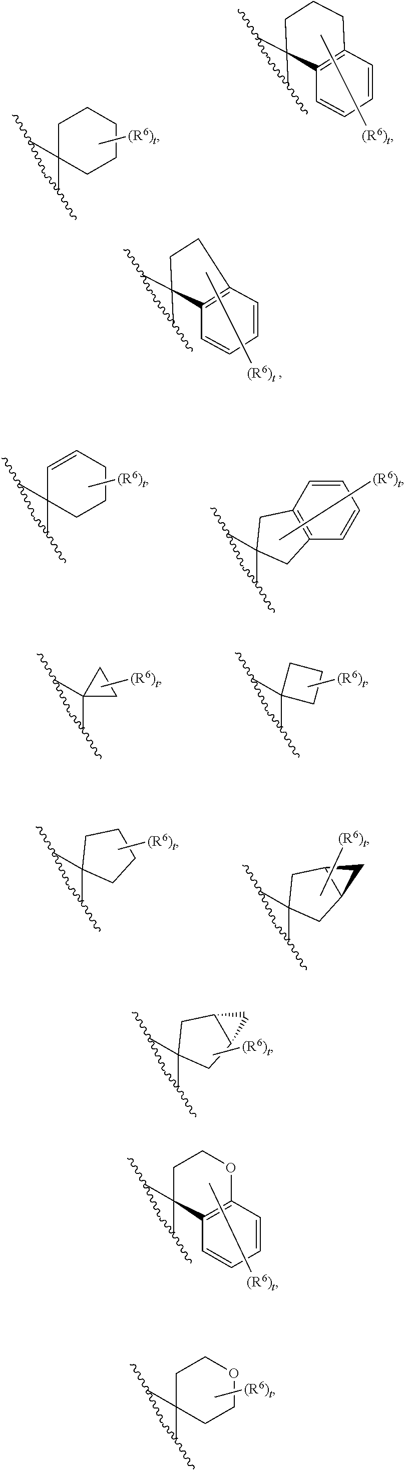

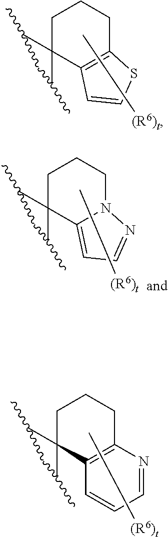

In some embodiments, Ring C together with its R⁶ substituents is

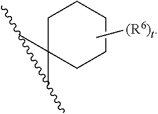

In some embodiments, Ring C together with its R⁶ substituents is

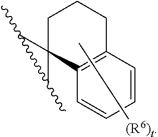

In some embodiments, Ring C together with its R⁶ substituents is

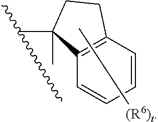

In some embodiments, Ring C together with its R⁶ substituents is

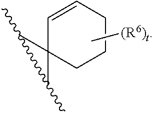

In some embodiments, Ring C together with its R⁶ substituents is

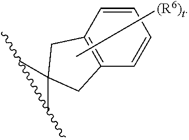

In some embodiments, Ring C together with its R⁶ substituents is

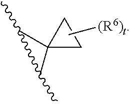

In some embodiments, Ring C together with its Re substituents is

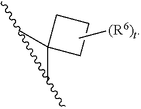

In some embodiments, Ring C together with its R⁶ substituents is

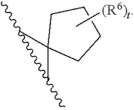

In some embodiments, Ring C together with its R⁶ substituents is

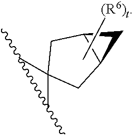

In some embodiments, Ring C together with its R⁶ substituents is

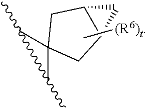

In some embodiments, Ring C together with its R⁶ substituents is

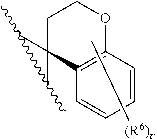

In some embodiments, Ring C together with its R⁶ substituents is

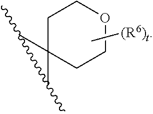

In some embodiments, Ring C together with its R⁶ substituents is

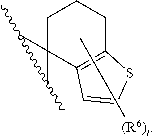

In some embodiments, Ring C together with its Re substituents is

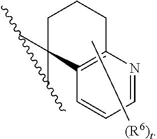

In some embodiments, Ring C together with its Re substituents is

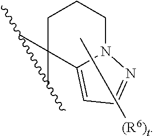

As defined generally above, L is a bivalent moiety that connects ERBM to LBM.

As defined generally above, L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

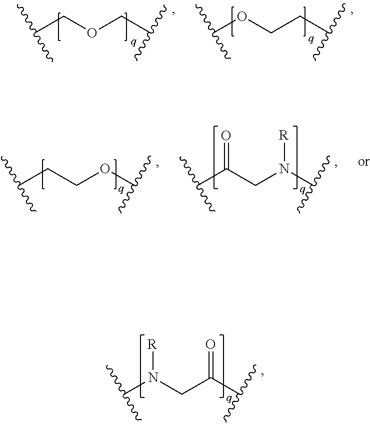

wherein each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

In some embodiments, L is a covalent bond. In some embodiments, L is a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

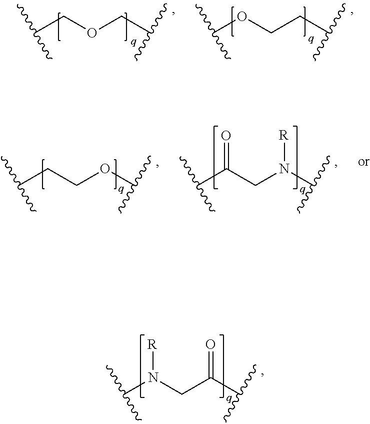

wherein each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

In some embodiments, L is a bivalent, saturated or unsaturated, straight or branched C₃₋₅ hydrocarbon chain, wherein 0, 1, 2 or 3 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

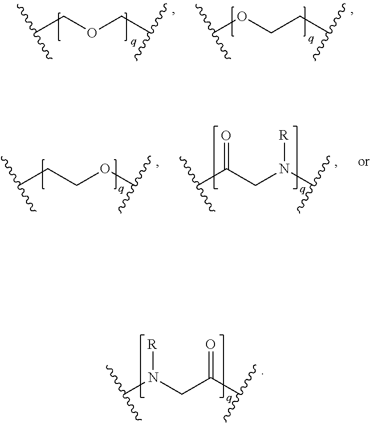

In some embodiments, L is a bivalent, saturated or unsaturated, straight or branched C₃₋₅ hydrocarbon chain, wherein 1, 2 or 3 methylene units of L are independently replaced by -Cy-, —CH(R)—, —C(R)₂—, —O— or —NR—.

In some embodiments, L is a bivalent, saturated or unsaturated, straight or branched C₃₋₅ hydrocarbon chain, wherein 1, 2 or 3 methylene units of L are independently replaced by -Cy- or —NR—.

In some embodiments, each -Cy- is independently an optionally substituted bivalent ring selected from a 4-7 membered saturated or partially unsaturated monocyclic carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated monocyclic heterocyclylenyl having 1-2 heteroatoms independently selected from nitrogen, oxygen, and sulfur, a 5-11 membered monocyclic saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur and a 6-10 membered bridged bicyclic saturated or partially unsaturated heterocyclylenyl having 1-2 heteroatoms independently selected from nitrogen, oxygen, and sulfur.

In some embodiments, each -Cy- is independently an optionally substituted bivalent ring selected from a 4-7 membered saturated or partially unsaturated monocyclic carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated monocyclic heterocyclylenyl containing 1-2 nitrogen atoms, a 5-11 membered monocyclic saturated or partially unsaturated spiro heterocyclylenyl containing 1-2 nitrogen atoms, an 8-10 membered bicyclic saturated or partially unsaturated heterocyclylenyl containing 1-2 nitrogen atoms, and a 6-10 membered bridged bicyclic saturated or partially unsaturated heterocyclylenyl containing 1-2 nitrogen atoms.

In some embodiments, the bivalent ring of each -Cy- is independently substituted with 0, 1 or 2 substituents independently selected from deuterium, C₁-C₄ alkyl, C₁-C₄ haloalkyl, C₃-C₇ cycloalkyl, —O—C₁-C₄ alkyl, halo, cyano, —OH, —NH₂, —N(H)(C₁-C₄ alkyl) and —N(C₁-C₄ alkyl)₂.

In some embodiments, the bivalent ring of each -Cy- is independently substituted with 0, 1 or 2 substituents independently selected from C₁-C₄ alkyl, C₁-C₄ haloalkyl, C₃-C₇ cycloalkyl, —O—C₁-C₄ alkyl, halo, cyano, —OH, —NH₂, —N(H) (C₁-C₄ alkyl) and —N(C₁-C₄ alkyl)₂.

In some embodiments, the bivalent ring of each -Cy- is independently substituted with 0, 1 or 2 substituents independently selected from deuterium, -Me, -Et, —Pr, -iPr, cyclopropyl, —CF₃, —OMe, —F, —Cl, —CN, —NH₂, —NHMe and —NMe₂.

In some embodiments, the bivalent ring of each -Cy- is independently substituted with 0, 1 or 2 substituents independently selected from -Me, -Et, —Pr, -iPr, cyclopropyl, —CF₃, —OMe, —F, —Cl, —CN, —NH₂, —NHMe and —NMe₂.

In some embodiments, the bivalent ring of each -Cy- is independently substituted with 0, 1 or 2 substituents independently selected from -Me, —OMe and —F.

In some embodiments, the bivalent ring of each -Cy- is independently substituted with 0, 1 or 2 instances of -Me.

In some embodiments, the bivalent ring of each -Cy- is unsubstituted.

In some embodiments, L is

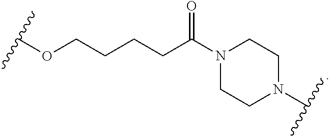 [SMILES: *OCCCCC(=O)N1CCN(*)CC1]

In some embodiments, L is selected from

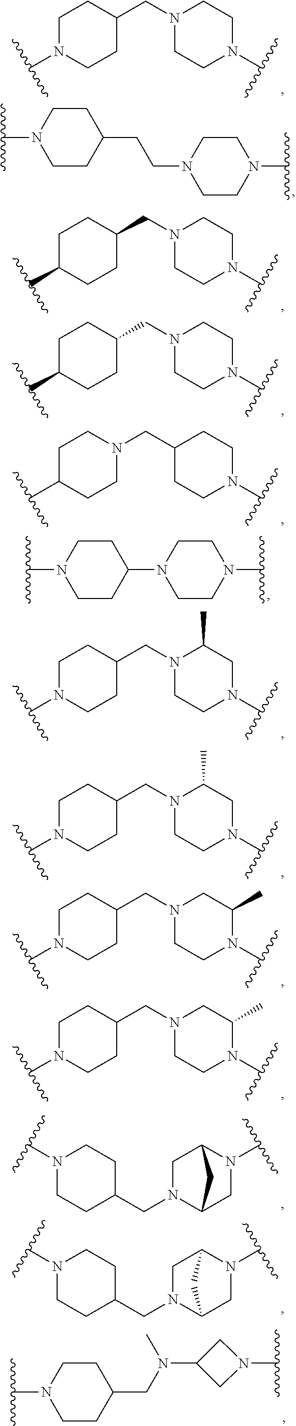

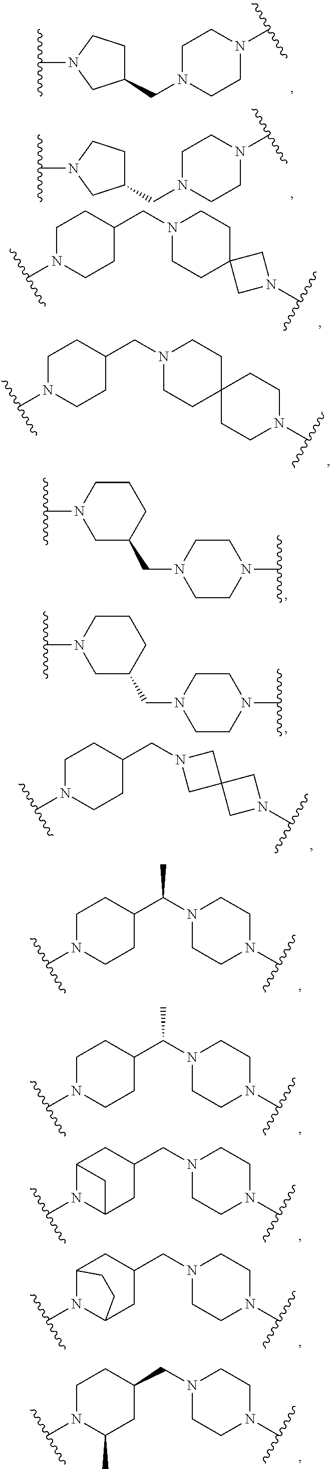

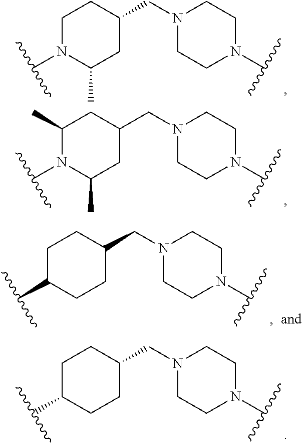

In some embodiments, L is selected from

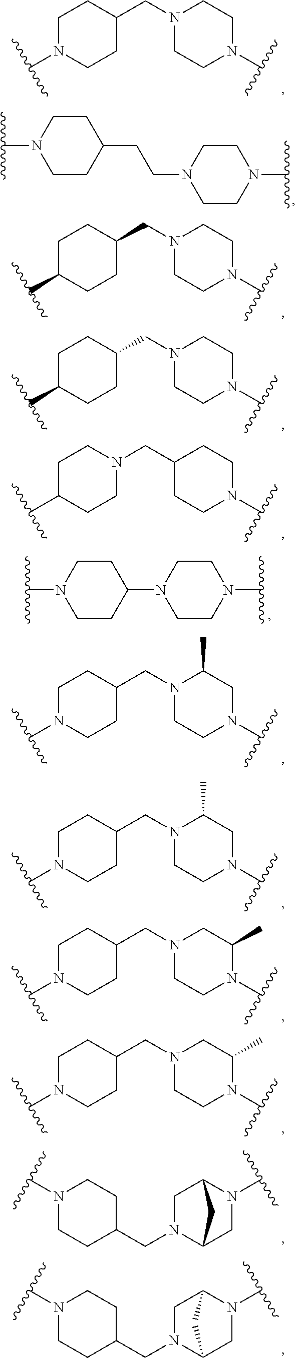

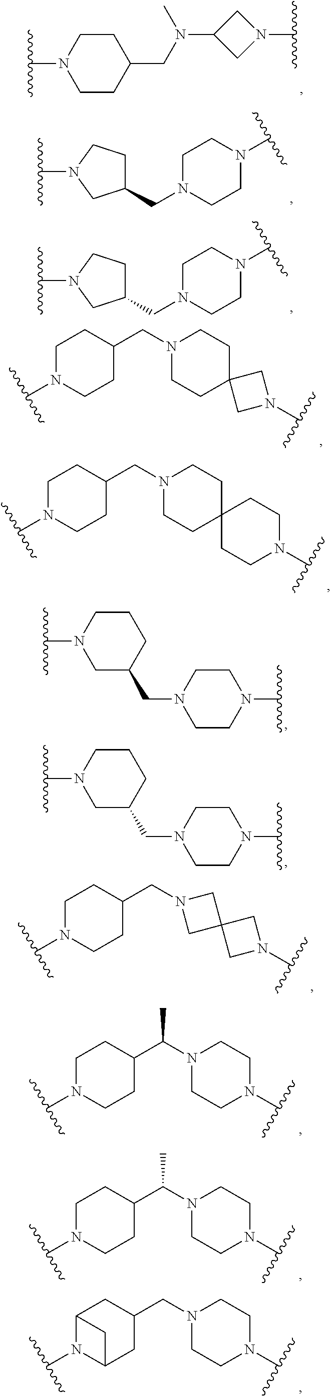

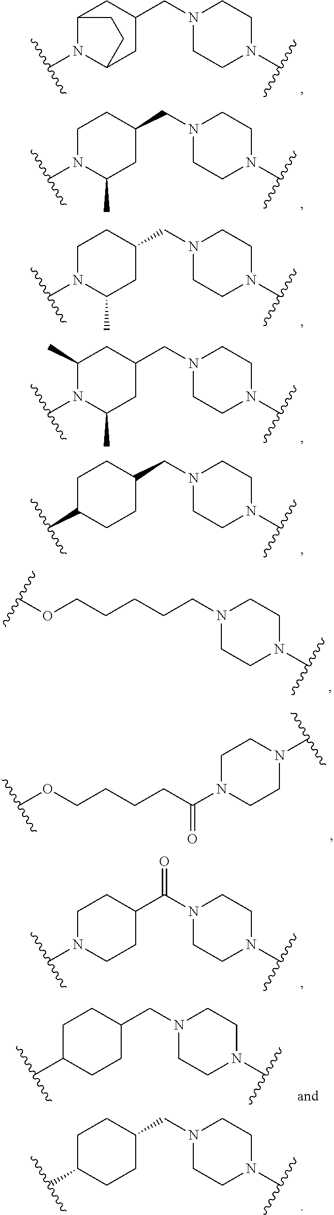

In some embodiments, L is selected from

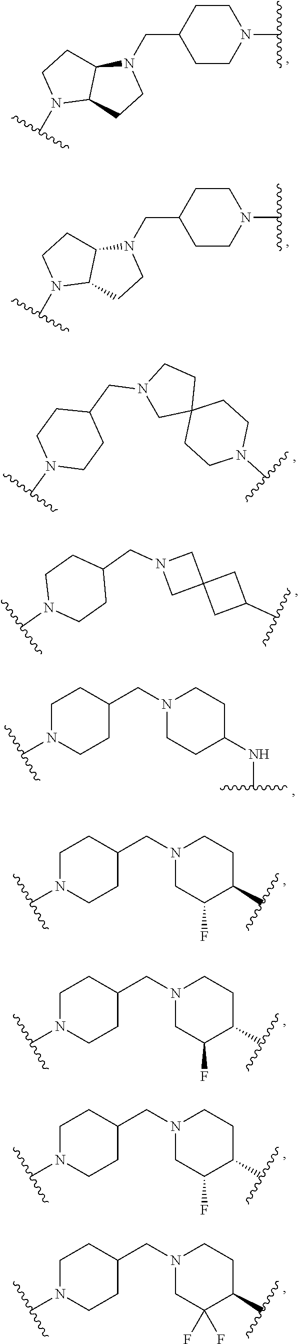

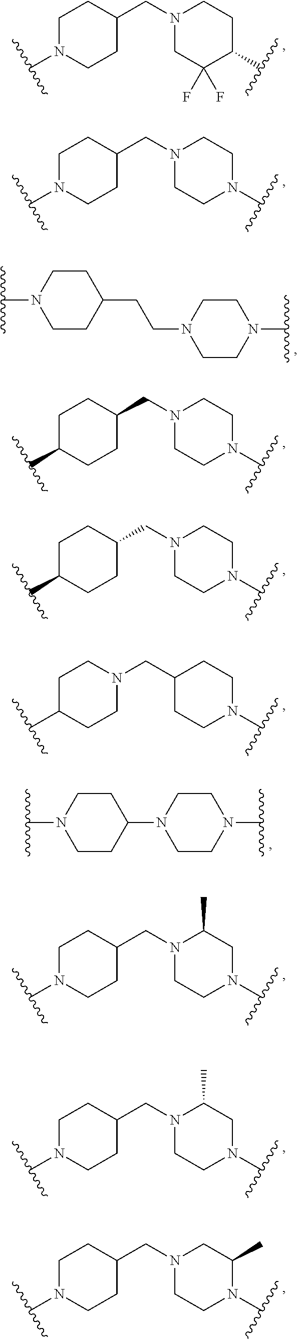

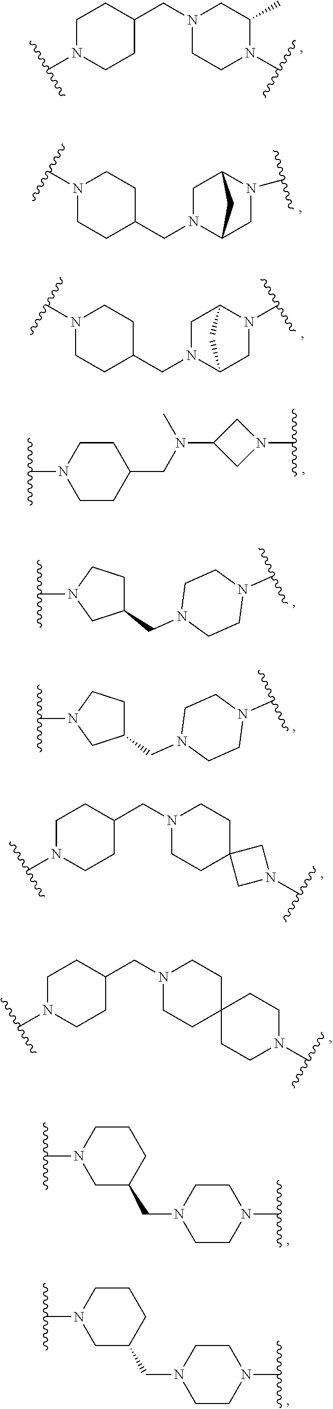

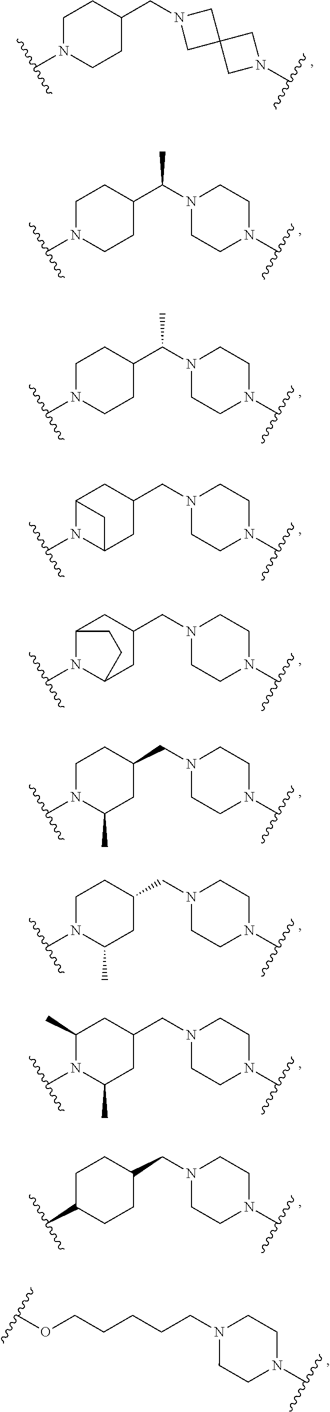

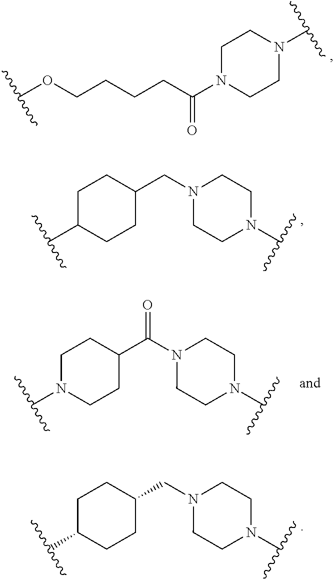 [SMILES: *OCCCCC(=O)N1CCN(*)CC1 ; *C1CCC(CN2CCN(*)CC2)CC1 ; *N1CCC(C(=O)N2CCN(*)CC2)CC1 ; *N1CCN(C[C@H]2CC[C@@H](*)CC2)CC1]

In some embodiments, L is selected from

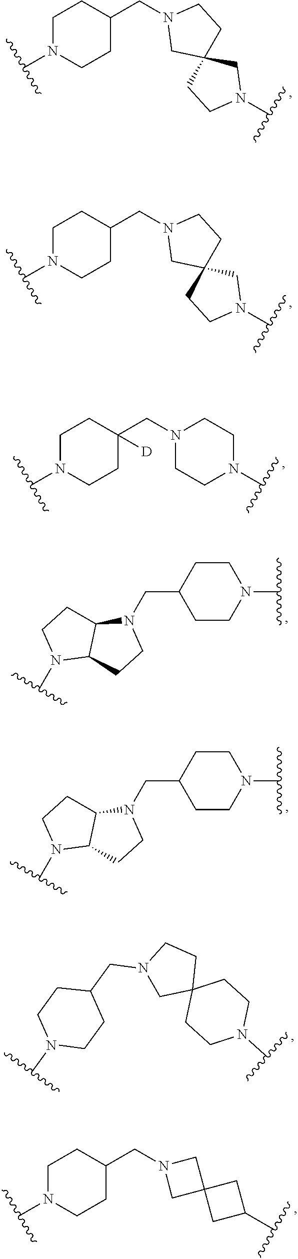

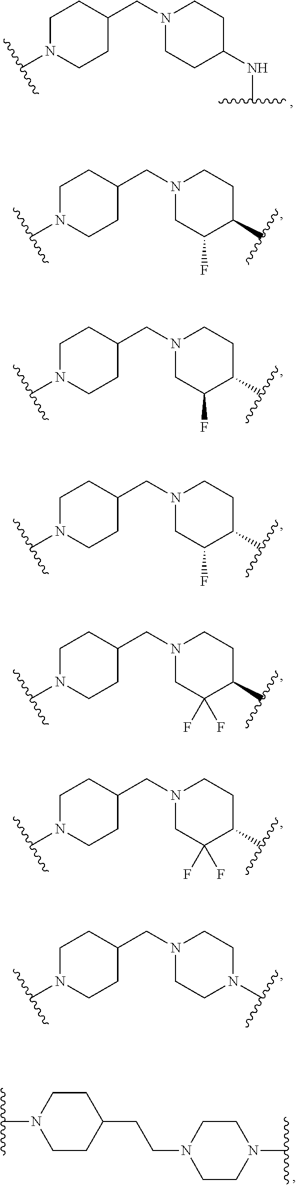

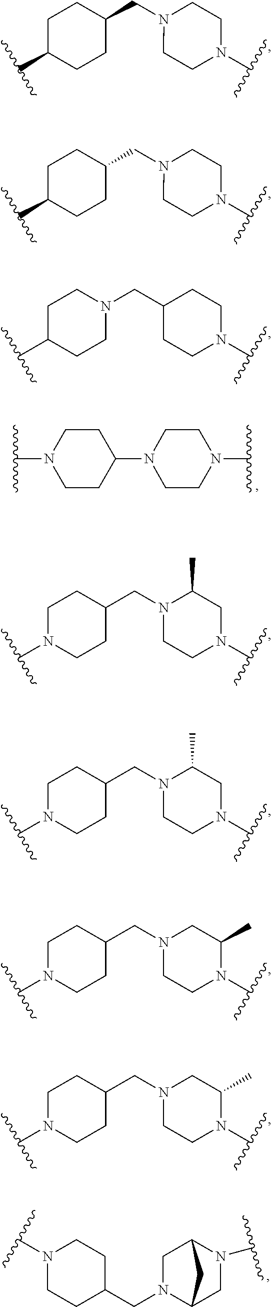

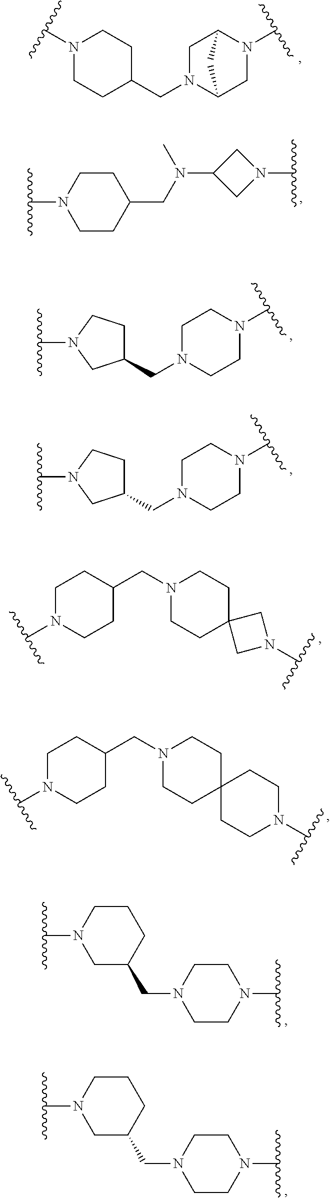

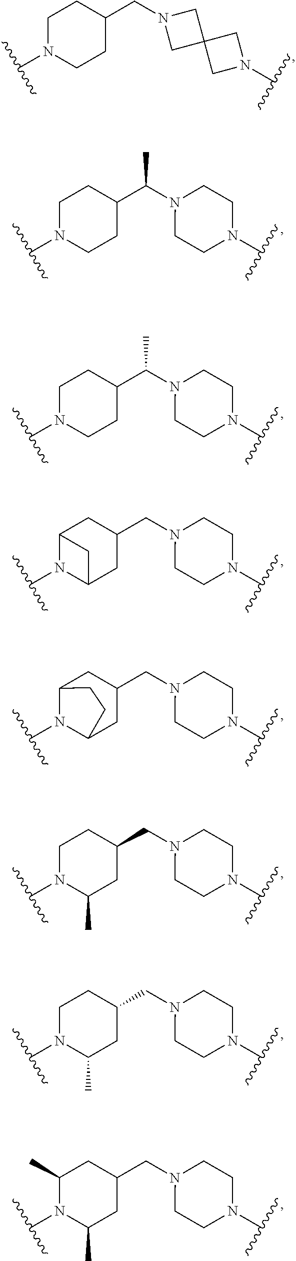

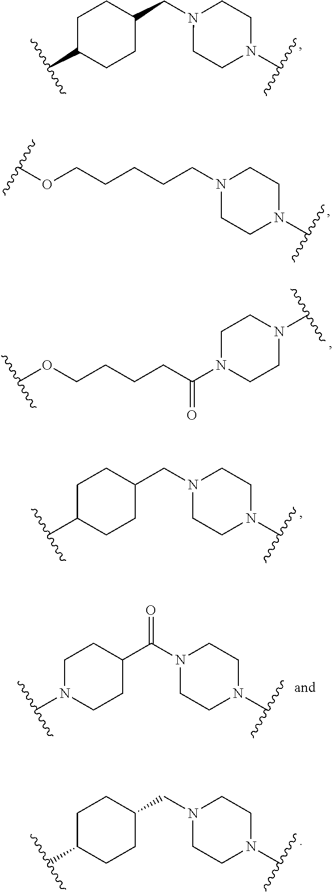

In some embodiments, L is

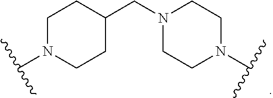 [SMILES: *N1CCC(CN2CCN(*)CC2)CC1]

In some embodiments, L is

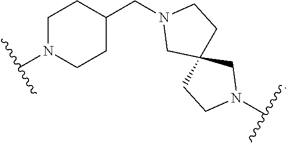 [SMILES: *N1CCC(CN2CC[C@]3(CCN(*)C3)C2)CC1]

In some embodiments, L is

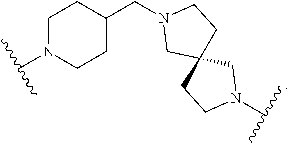 [SMILES: *N1CCC(CN2CC[C@@]3(CCN(*)C3)C2)CC1]

In some embodiments, L is

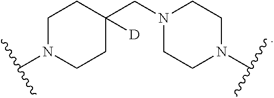 [SMILES: *N1CCN(CC2([2H])CCN(*)CC2)CC1]

In some embodiments, L is

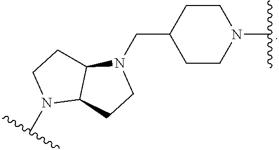 [SMILES: *N1CCC(CN2CC[C@@H]3[C@H]2CCN3*)CC1]

In some embodiments, L is

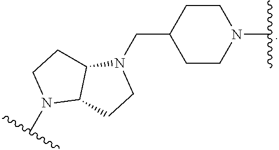 [SMILES: *N1CCC(CN2CC[C@H]3[C@@H]2CCN3*)CC1]

In some embodiments, L is

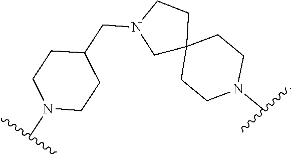 [SMILES: *N1CCC(CN2CCC3(CCN(*)CC3)C2)CC1]

In some embodiments, L is

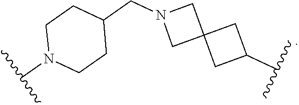 [SMILES: *C1CC2(C1)CN(CC1CCN(*)CC1)C2]

In some embodiments, L is

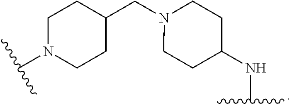 [SMILES: *NC1CCN(CC2CCN(*)CC2)CC1]

In some embodiments, L is

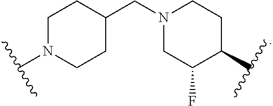 [SMILES: *[C@@H]1CCN(CC2CCN(*)CC2)C[C@H]1F]

In some embodiments, L is

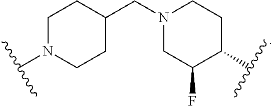 [SMILES: *[C@H]1CCN(CC2CCN(*)CC2)C[C@@H]1F]

In some embodiments, L is

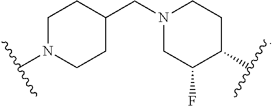 [SMILES: *[C@H]1CCN(CC2CCN(*)CC2)C[C@H]1F]

In some embodiments, L is

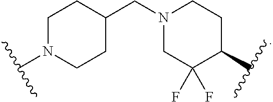 [SMILES: *[C@@H]1CCN(CC2CCN(*)CC2)CC1(F)F]

In some embodiments, L is

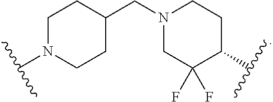 [SMILES: *[C@H]1CCN(CC2CCN(*)CC2)CC1(F)F]

In some embodiments, L is

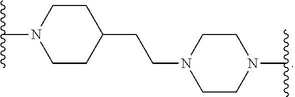 [SMILES: *N1CCC(CCN2CCN(*)CC2)CC1]

In some embodiments, L is

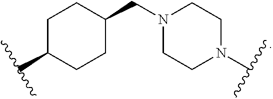 [SMILES: *N1CCN(C[C@H]2CC[C@@H](*)CC2)CC1]

In some embodiments, L is

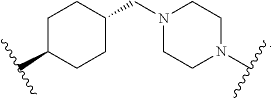 [SMILES: *N1CCN(C[C@H]2CC[C@H](*)CC2)CC1]

In some embodiments, L is

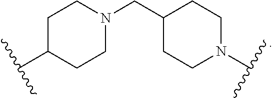 [SMILES: *C1CCN(CC2CCN(*)CC2)CC1]

In some embodiments, L is

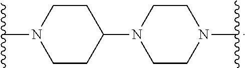 [SMILES: *N1CCC(N2CCN(*)CC2)CC1]

In some embodiments, L is

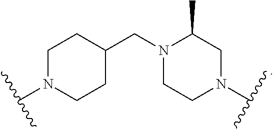 [SMILES: *N1CCC(CN2CCN(*)C[C@@H]2C)CC1]

In some embodiments, L is

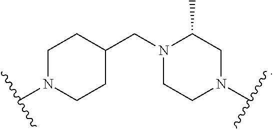 [SMILES: *N1CCC(CN2CCN(*)C[C@H]2C)CC1]

In some embodiments, L is

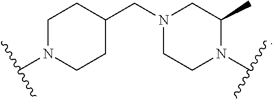 [SMILES: *N1CCC(CN2CCN(*)[C@H](C)C2)CC1]

In some embodiments, L is

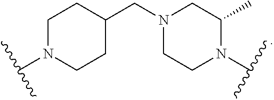 [SMILES: *N1CCC(CN2CCN(*)[C@@H](C)C2)CC1]

In some embodiments, L is

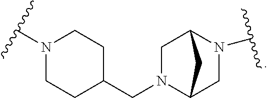 [SMILES: *N1CCC(CN2C[C@H]3C[C@@H]2CN3*)CC1]

In some embodiments, L is

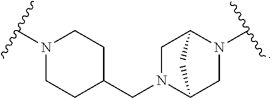 [SMILES: *N1CCC(CN2C[C@@H]3C[C@H]2CN3*)CC1]

In some embodiments, L is

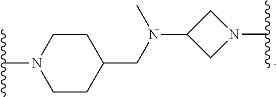 [SMILES: *N1CCC(CN(C)C2CN(*)C2)CC1]

In some embodiments, L is

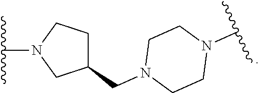 [SMILES: *N1CCN(C[C@@H]2CCN(*)C2)CC1]

In some embodiments, L is

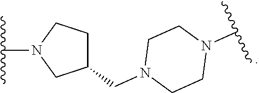 [SMILES: *N1CCN(C[C@H]2CCN(*)C2)CC1]

In some embodiments, L is

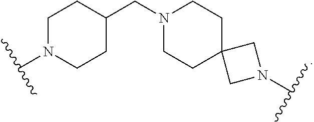 [SMILES: *N1CCC(CN2CCC3(CC2)CN(*)C3)CC1]

In some embodiments, L is

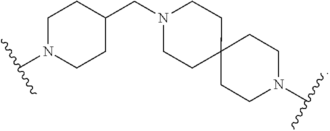 [SMILES: *N1CCC(CN2CCC3(CCN(*)CC3)CC2)CC1]

In some embodiments, L is

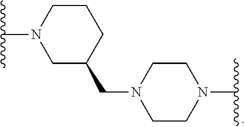 [SMILES: *N1CCN(C[C@@H]2CCCN(*)C2)CC1]

In some embodiments, L is

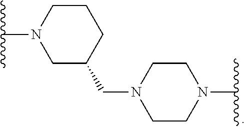 [SMILES: *N1CCN(C[C@H]2CCCN(*)C2)CC1]

In some embodiments, L is

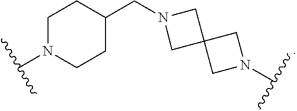 [SMILES: *N1CCC(CN2CC3(CN(*)C3)C2)CC1]

In some embodiments, L is

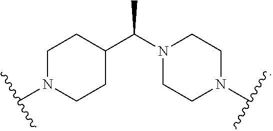 [SMILES: *N1CCC([C@@H](C)N2CCN(*)CC2)CC1]

In some embodiments, L is

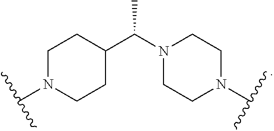 [SMILES: *N1CCC([C@H](C)N2CCN(*)CC2)CC1]

In some embodiments, L is

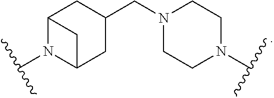 [SMILES: *N1CCN(CC2CC3CC(C2)N3*)CC1]

In some embodiments, L is

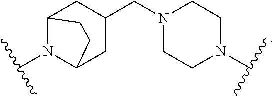 [SMILES: *N1CCN(CC2CC3CCC(C2)N3*)CC1]

In some embodiments, L is

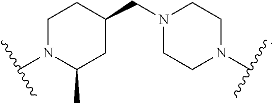 [SMILES: *N1CCN(C[C@@H]2CCN(*)[C@H](C)C2)CC1]

In some embodiments, L is

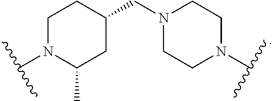 [SMILES: *N1CCN(C[C@H]2CCN(*)[C@@H](C)C2)CC1]

In some embodiments, L is

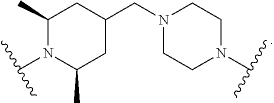 [SMILES: *N1CCN(CC2C[C@@H](C)N(*)[C@@H](C)C2)CC1]

In some embodiments, L is

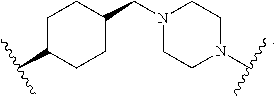

In some embodiments, L is

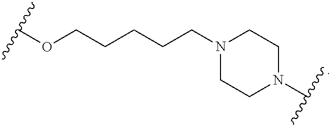 [SMILES: *OCCCCCN1CCN(*)CC1]

In some embodiments, L is

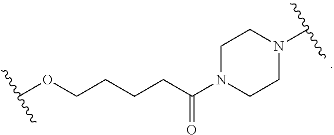 [SMILES: *OCCCCC(=O)N1CCN(*)CC1]

In some embodiments, L is

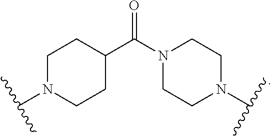 [SMILES: *N1CCC(C(=O)N2CCN(*)CC2)CC1]

In some embodiments, L is

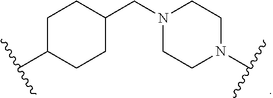 [SMILES: *C1CCC(CN2CCN(*)CC2)CC1]

In some embodiments, L is

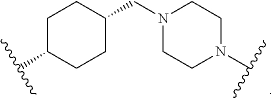 [SMILES: *N1CCN(C[C@H]2CC[C@@H](*)CC2)CC1]

In some embodiments, L is selected from the groups depicted in the compounds in Table 1 or Table 2.

As defined generally above, LBM is a ligase binding moiety.

As defined generally above, LBM is selected from

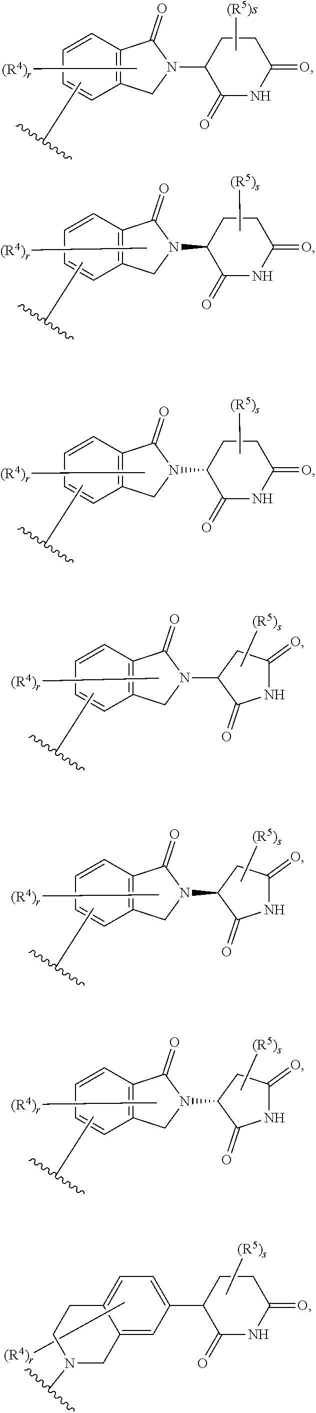

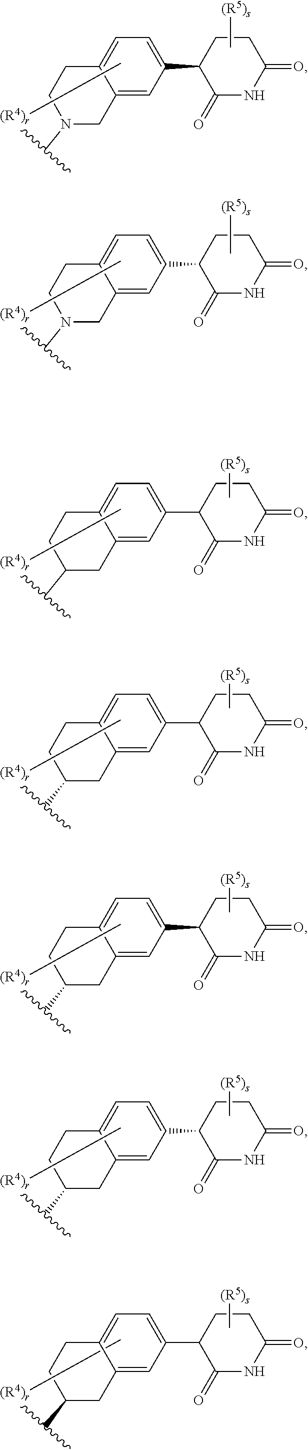

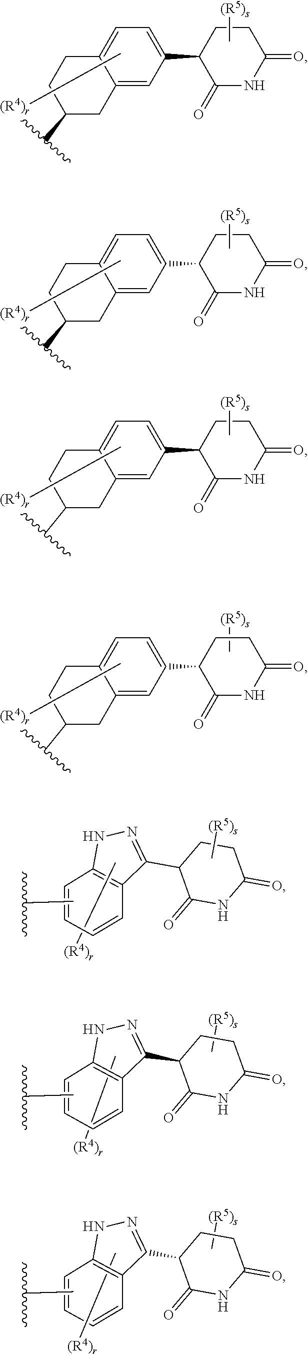

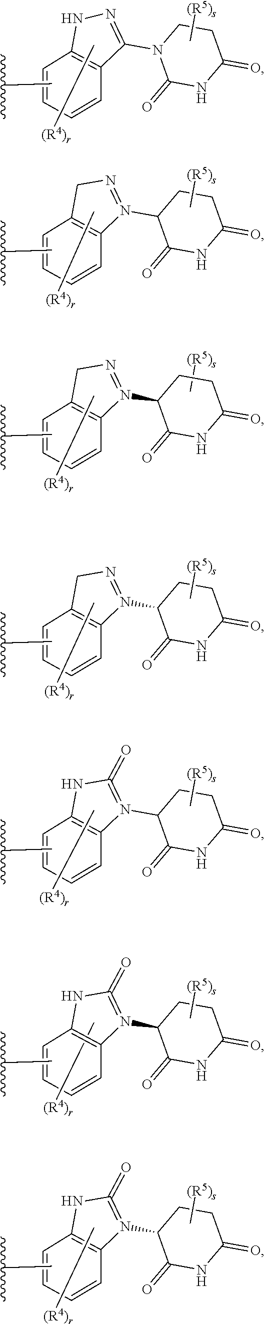

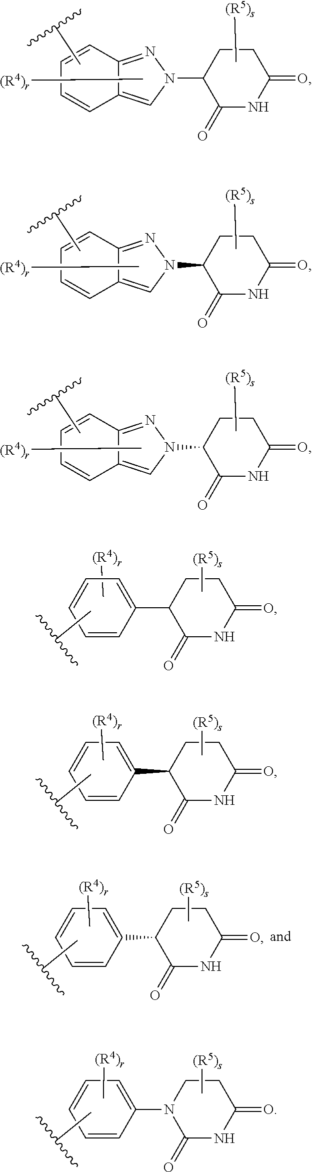

In some embodiments, LBM is selected from

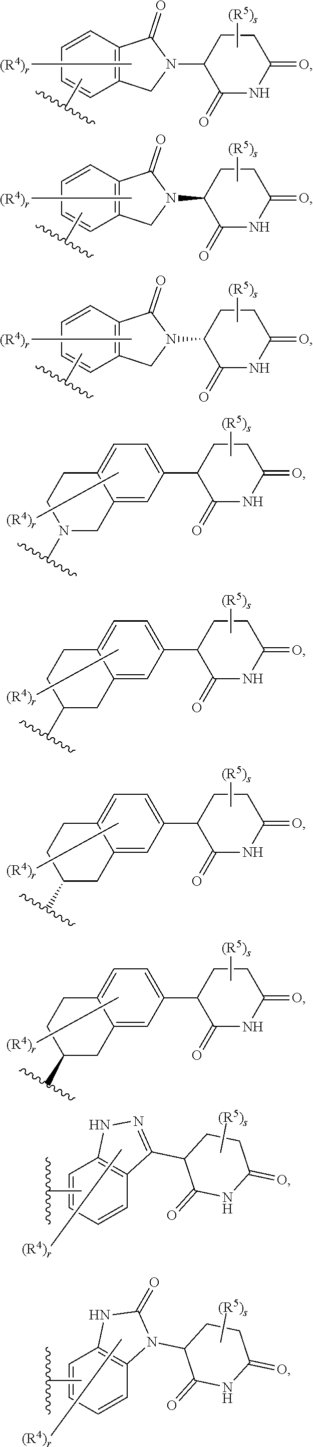

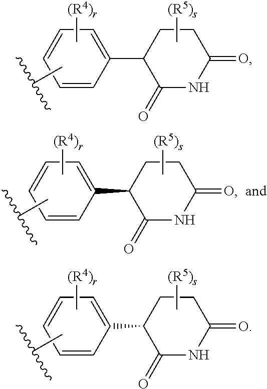

In some embodiments, LBM is selected from

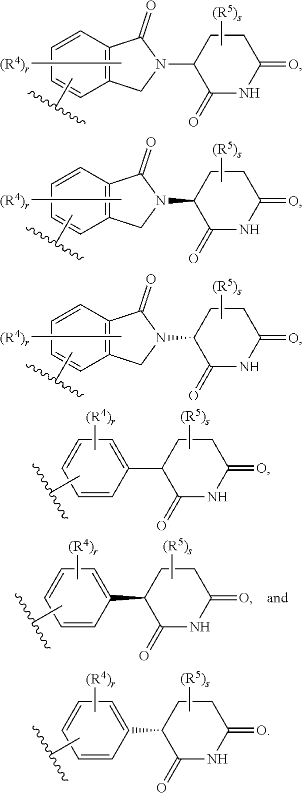

In some embodiments, LBM is selected from

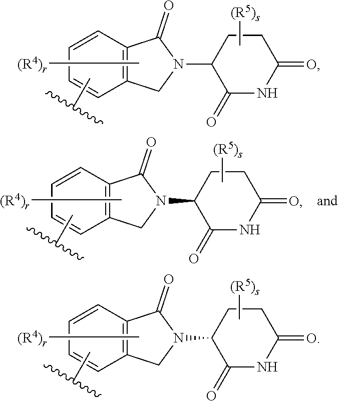

In some embodiments, LBM is selected from

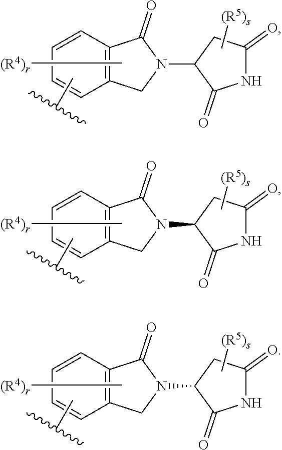

In some embodiments, LBM is selected from

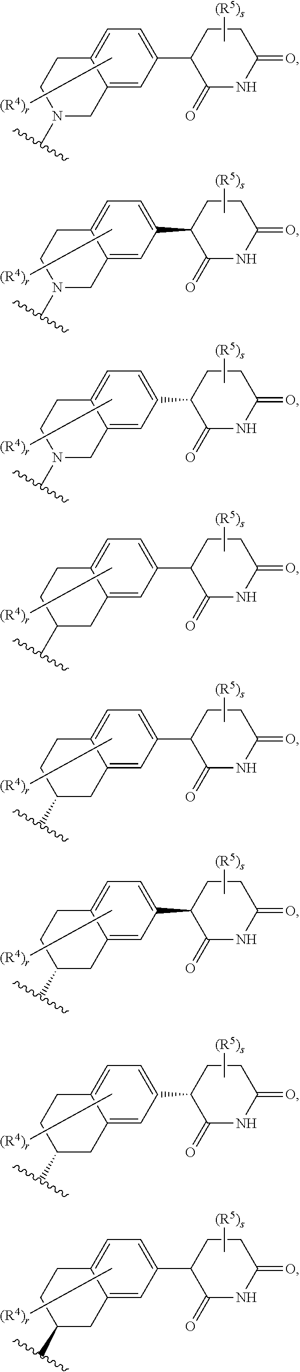

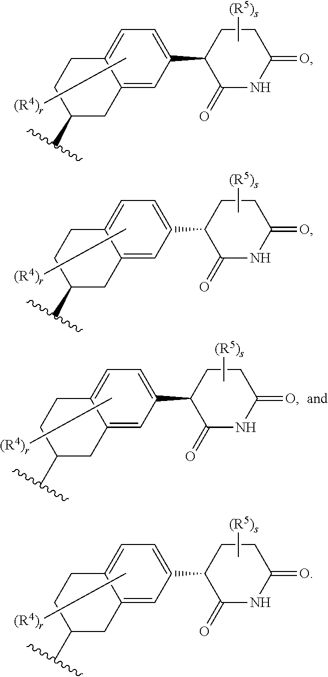

some embodiments, LBM is selected from

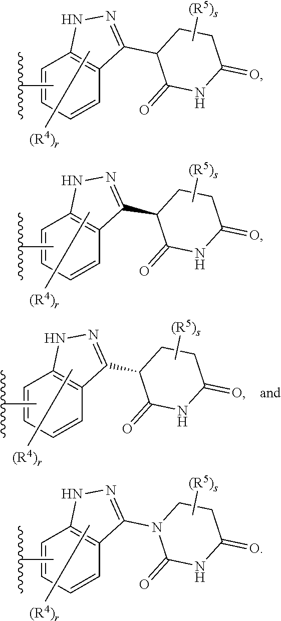

some embodiments, LBM is selected from

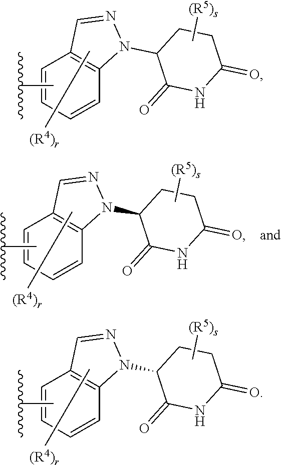

some embodiments, LBM is selected from

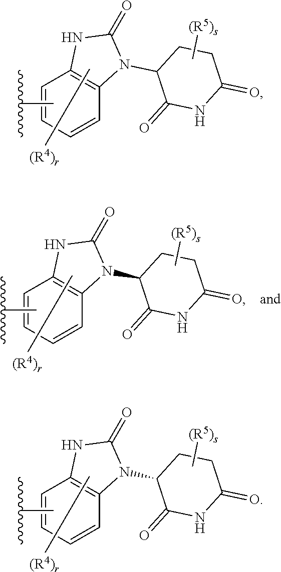

some embodiments, LBM is selected from

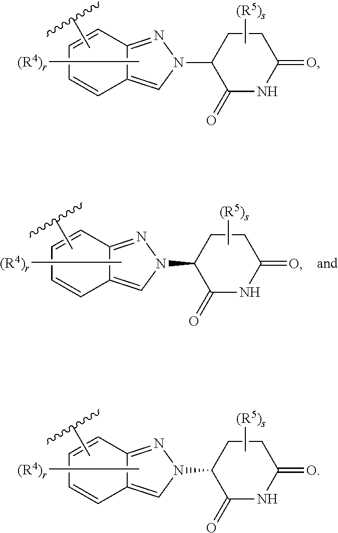

some embodiments, LBM is selected from

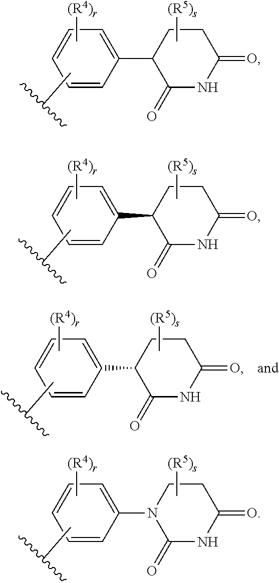

In some embodiments, LBM is selected from

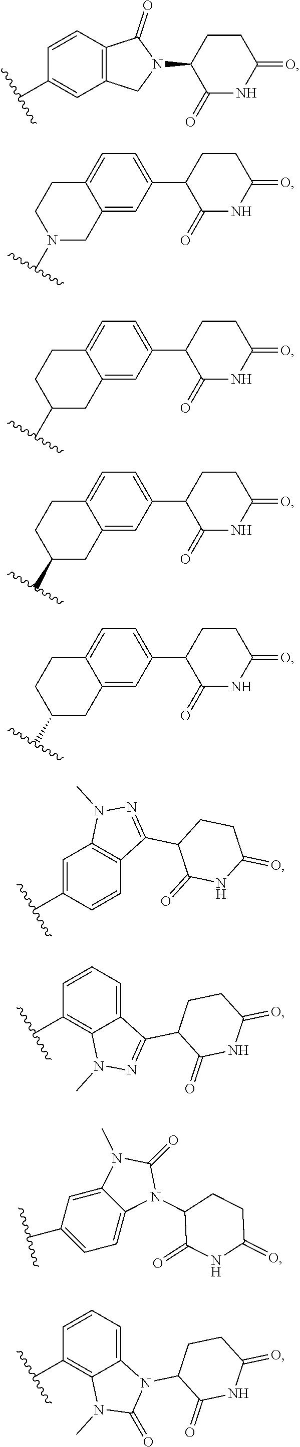

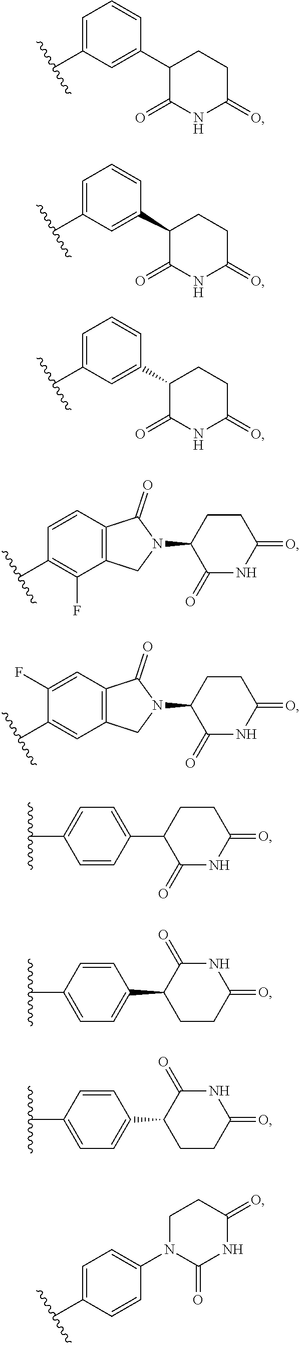

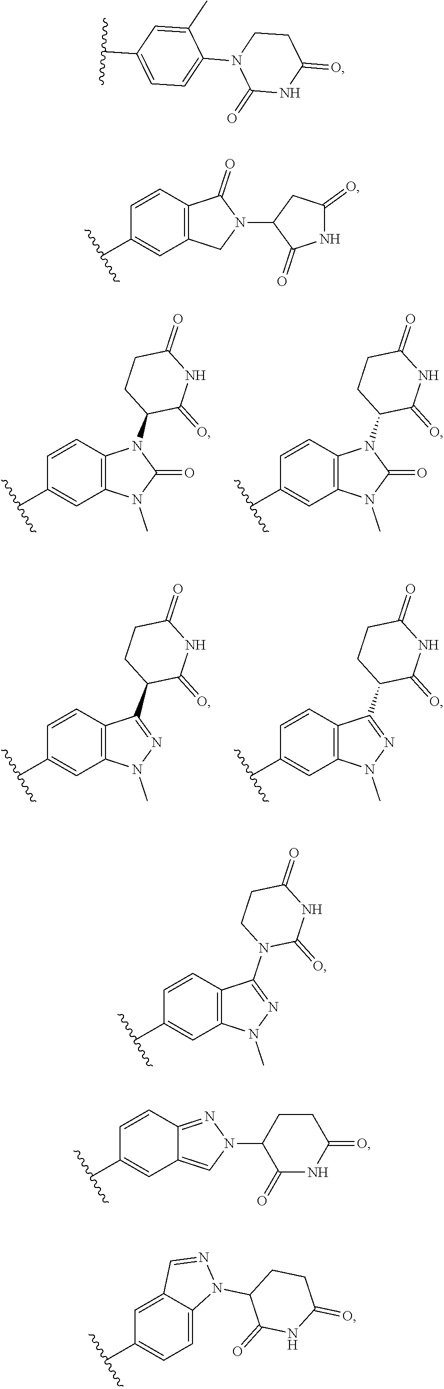

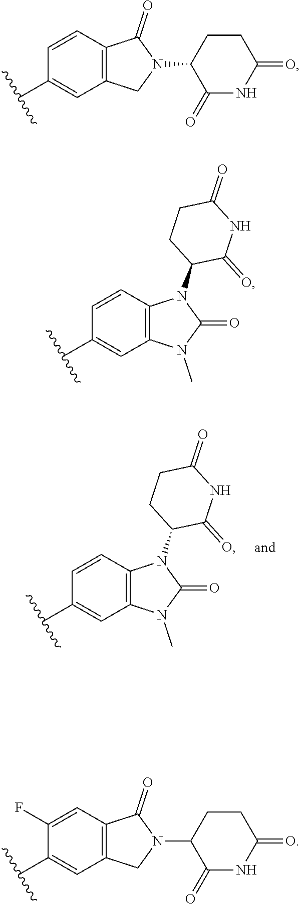 [SMILES: *c1ccc2c(c1)CN([C@@H]1CCC(=O)NC1=O)C2=O ; *c1ccc2c(c1)n(C)c(=O)n2[C@H]1CCC(=O)NC1=O ; *c1ccc2c(c1)n(C)c(=O)n2[C@@H]1CCC(=O)NC1=O ; "and" ; *c1cc2c(cc1F)C(=O)N(C1CCC(=O)NC1=O)C2]

In some embodiments, LBM is selected from

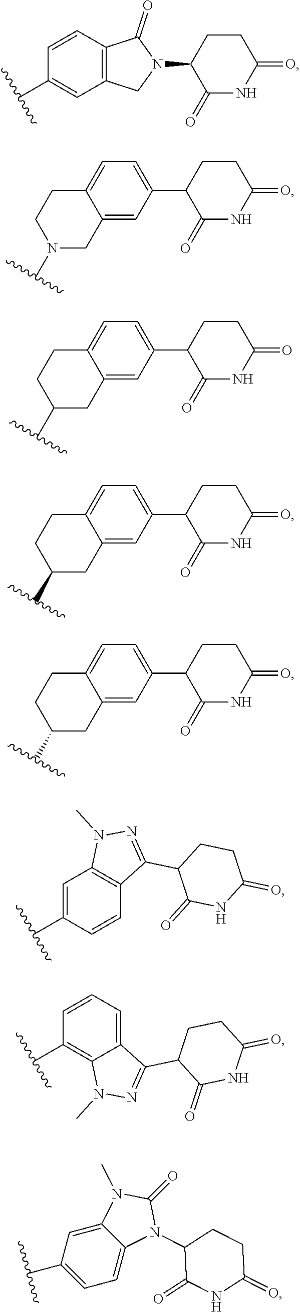

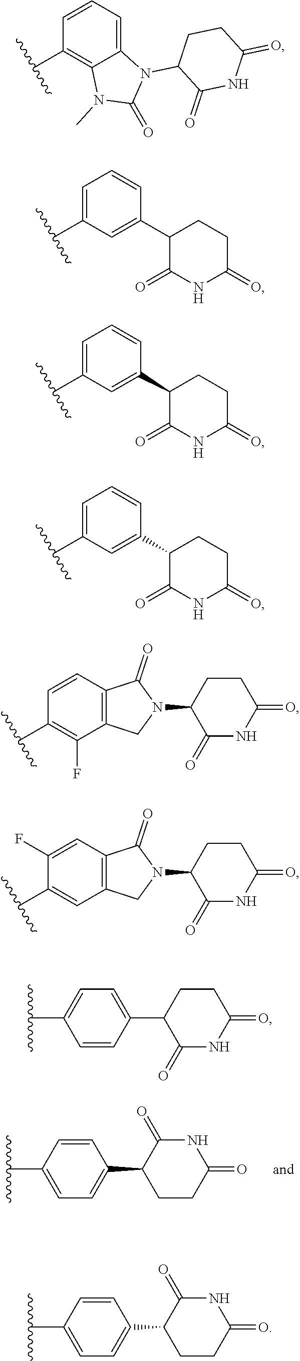

In some embodiments, LBM is selected from

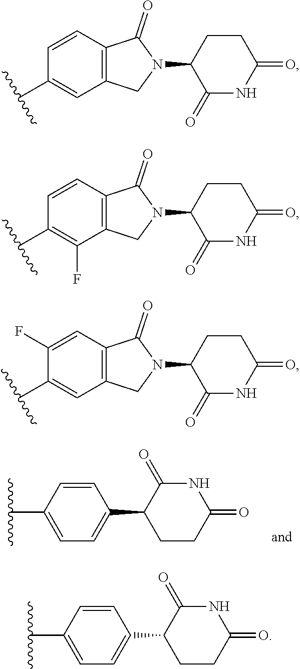

In some embodiments, LBM is selected from

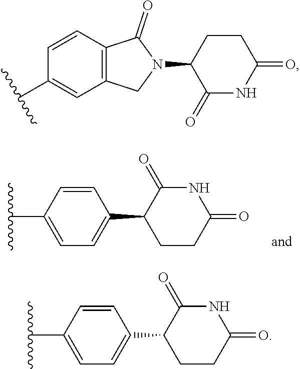

In some embodiments, LBM is

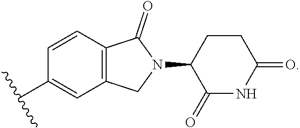 [SMILES: *c1ccc2c(c1)CN([C@H]1CCC(=O)NC1=O)C2=O]

In some embodiments, LBM is

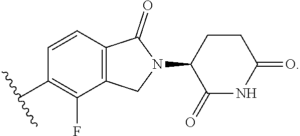 [SMILES: *c1ccc2c(c1F)CN([C@H]1CCC(=O)NC1=O)C2=O]

In some embodiments, LBM is

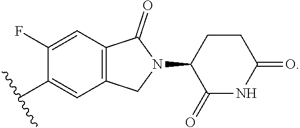 [SMILES: *c1cc2c(cc1F)C(=O)N([C@H]1CCC(=O)NC1=O)C2]

In some embodiments, LBM is

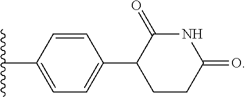 [SMILES: *c1ccc(C2CCC(=O)NC2=O)cc1]

In some embodiments, LBM is

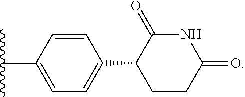 [SMILES: *c1ccc([C@H]2CCC(=O)NC2=O)cc1]

In some embodiments, LBM is

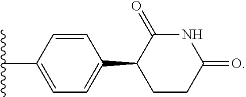 [SMILES: *c1ccc([C@@H]2CCC(=O)NC2=O)cc1]

In some embodiments, LBM is

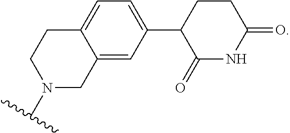 [SMILES: *N1CCc2ccc(C3CCC(=O)NC3=O)cc2C1]

In some embodiments, LBM is

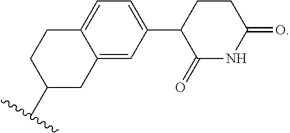 [SMILES: *C1CCc2ccc(C3CCC(=O)NC3=O)cc2C1]

In some embodiments, LBM is

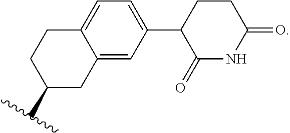 [SMILES: *[C@H]1CCc2ccc(C3CCC(=O)NC3=O)cc2C1]

In some embodiments, LBM is

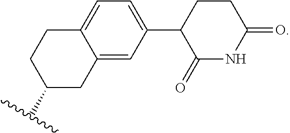 [SMILES: *[C@@H]1CCc2ccc(C3CCC(=O)NC3=O)cc2C1]

In some embodiments, LBM is

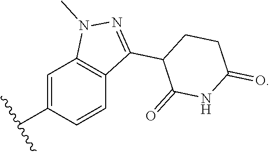 [SMILES: *c1ccc2c(C3CCC(=O)NC3=O)nn(C)c2c1]

In some embodiments, LBM is

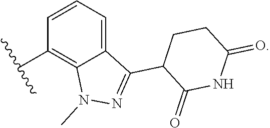 [SMILES: *c1cccc2c(C3CCC(=O)NC3=O)nn(C)c12]

In some embodiments, LBM is

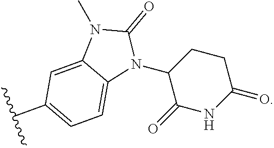 [SMILES: *c1ccc2c(c1)n(C)c(=O)n2C1CCC(=O)NC1=O]

In some embodiments, LBM is

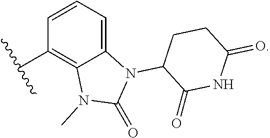 [SMILES: *c1cccc2c1n(C)c(=O)n2C1CCC(=O)NC1=O]

In some embodiments, LBM is

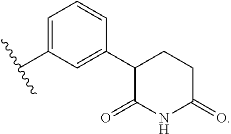 [SMILES: *c1cccc(C2CCC(=O)NC2=O)c1]

In some embodiments, LBM is

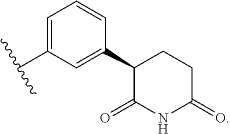 [SMILES: *c1cccc([C@H]2CCC(=O)NC2=O)c1]

In some embodiments, LBM is

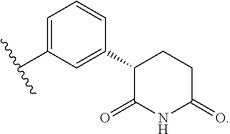 [SMILES: *c1cccc([C@@H]2CCC(=O)NC2=O)c1]

In some embodiments, LBM is

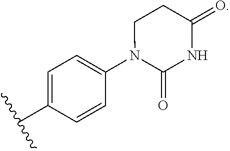 [SMILES: *c1ccc(N2CCC(=O)NC2=O)cc1]

In some embodiments, LBM is

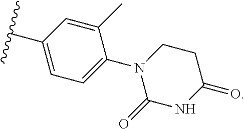 [SMILES: *c1ccc(N2CCC(=O)NC2=O)c(C)c1]

In some embodiments, LBM is

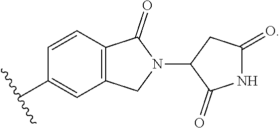 [SMILES: *c1ccc2c(c1)CN(C1CC(=O)NC1=O)C2=O]

In some embodiments, LBM is

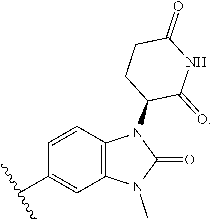 [SMILES: *c1ccc2c(c1)n(C)c(=O)n2[C@H]1CCC(=O)NC1=O]

In some embodiments, LBM is

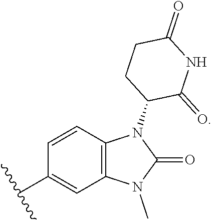 [SMILES: *c1ccc2c(c1)n(C)c(=O)n2[C@@H]1CCC(=O)NC1=O]

In some embodiments, LBM is

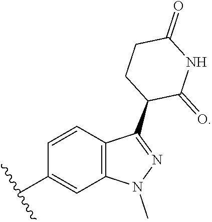 [SMILES: *c1ccc2c([C@H]3CCC(=O)NC3=O)nn(C)c2c1]

In some embodiments, LBM is

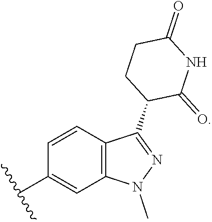 [SMILES: *c1ccc2c([C@@H]3CCC(=O)NC3=O)nn(C)c2c1]

In some embodiments, LBM is

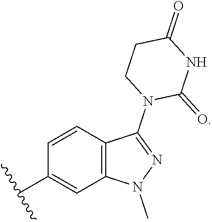 [SMILES: *c1ccc2c(N3CCC(=O)NC3=O)nn(C)c2c1]

In some embodiments, LBM is

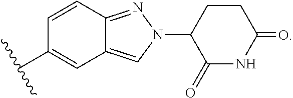 [SMILES: *c1ccc2nn(C3CCC(=O)NC3=O)cc2c1]

In some embodiments, LBM is

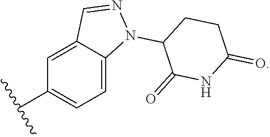 [SMILES: *c1ccc2c(cnn2C2CCC(=O)NC2=O)c1]

In some embodiments, LBM is

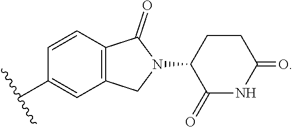 [SMILES: *c1ccc2c(c1)CN([C@@H]1CCC(=O)NC1=O)C2=O]

In some embodiments, LBM is

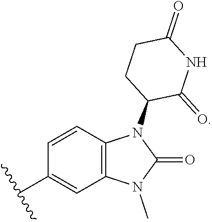 [SMILES: *c1ccc2c(c1)n(C)c(=O)n2[C@H]1CCC(=O)NC1=O]

In some embodiments, LBM is

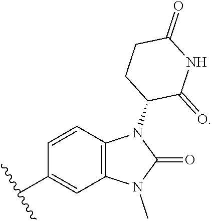 [SMILES: *c1ccc2c(c1)n(C)c(=O)n2[C@@H]1CCC(=O)NC1=O]

In some embodiments, LBM is

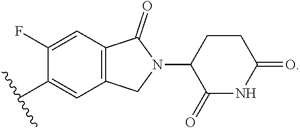 [SMILES: *c1cc2c(cc1F)C(=O)N(C1CCC(=O)NC1=O)C2]

In some embodiments, LBM is selected from the groups depicted in the compounds in Table 1 or Table 2.

As defined generally above, m is 0, 1, 2, 3, or 4. In some embodiments, m is 0, 1, 2 or 3.

In some embodiments, m is 0, 1 or 2. In some embodiments, m is 0 or 1.

In some embodiments m is 0. In some embodiments m is 1. In some embodiments m is 2. In some embodiments m is 3. In some embodiments m is 4.

In some embodiments, m is selected from the groups depicted in the compounds in Table 1 or Table 2.

As defined generally above, n is 0, 1, 2, 3, or 4. In some embodiments, n is 0, 1, 2 or 3. In some embodiments, n is 0, 1 or 2. In some embodiments, n is 0 or 1.

In some embodiments n is 0. In some embodiments n is 1. In some embodiments n is 2.

In some embodiments n is 3. In some embodiments n is 4.

In some embodiments, n is selected from the groups depicted in the compounds in Table 1 or Table 2.

As defined generally above, p is 0, 1, 2, 3, or 4. In some embodiments, p is 0, 1, 2 or 3. In some embodiments, p is 0, 1 or 2. In some embodiments, p is 0 or 1. Of note, when the number of R³ groups is designated as “p-1” or “p-2”, solely positive integers are contemplated (i.e., “p-1” can be 0, 1, 2 or 3 and “p-2” can be 0, 1 or 2).

In some embodiments p is 0. In some embodiments p is 1. In some embodiments p is 2.

In some embodiments p is 3. In some embodiments p is 4.

In some embodiments, p is selected from the groups depicted in the compounds in Table 1 or Table 2.

As defined generally above, each of q is independently 1, 2, 3, 4, 5, 6, 7, 8, 9, or 10.

In some embodiments q is 1. In some embodiments q is 2. In some embodiments q is 3.

In some embodiments q is 4. In some embodiments q is 5. In some embodiments q is 6. In some embodiments q is 7. In some embodiments q is 8. In some embodiments q is 9. In some embodiments q is 10.

In some embodiments, q is selected from the groups depicted in the compounds in Table 1 or Table 2.

As defined generally above, r is 0, 1, 2, 3, or 4. In some embodiments, r is 0, 1, 2 or 3. In some embodiments, r is 0, 1 or 2. In some embodiments, r is 0 or 1.

In some embodiments r is 0. In some embodiments r is 1. In some embodiments r is 2.

In some embodiments r is 3. In some embodiments r is 4.

In some embodiments, r is selected from the groups depicted in the compounds in Table 1 or Table 2.

As defined generally above, s is 0, 1, 2, 3, or 4. In some embodiments, s is 0, 1, 2 or 3. In some embodiments, s is 0, 1 or 2. In some embodiments, s is 0 or 1.

In some embodiments s is 0. In some embodiments s is 1. In some embodiments s is 2.

In some embodiments s is 3. In some embodiments s is 4.

In some embodiments, s is selected from the groups depicted in the compounds in Table 1 or Table 2.

In certain embodiments, the present invention provides a compound of formula I-1, I-1′, I-2, I-2′, I-3 or I-3′, wherein ERBM is

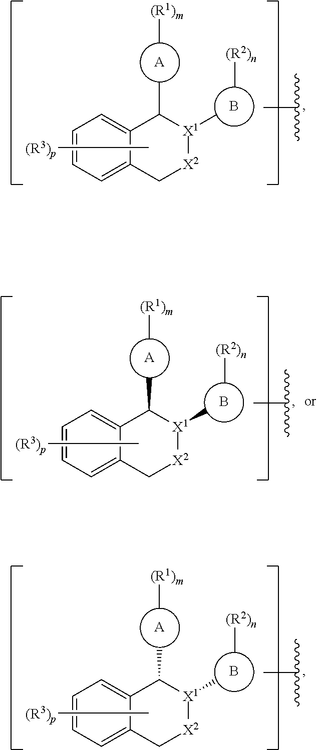

thereby forming a compound of one of formulas II-a, II-b, or II-c, respectively:

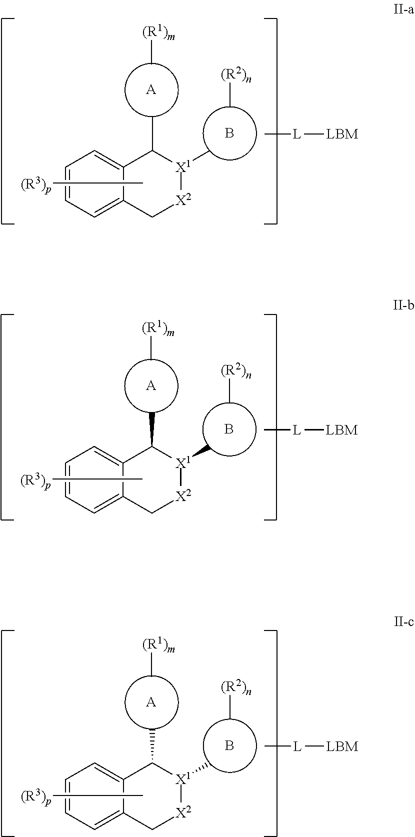

or a pharmaceutically acceptable salt thereof, wherein each of Ring A, Ring B, R¹, R², R³, X¹, X², m, n, p, L, and LBM is as defined in embodiments and classes and subclasses herein.

In some embodiments, when X¹ is CH or N, X² is CH and Ring B is phenyl, then ring A is other than phenyl or a 6-membered monocyclic heteroaryl ring containing 1-3 nitrogen heteroatoms.

In some embodiments, the compound is a compound of formula II-a-1, II-b-1, or II-c-1:

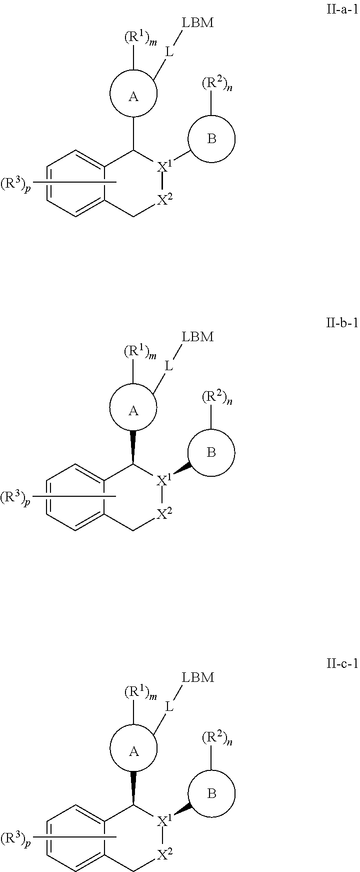

or a pharmaceutically acceptable salt thereof, wherein each of Ring A, Ring B, R¹, R², R³, X¹, X², m, n, p, L, and LBM is as defined in embodiments and classes and subclasses herein. In some embodiments, when X¹ is CH or N, X² is CH and Ring B is phenyl, then ring A is other than phenyl or a 6-membered monocyclic heteroaryl ring containing 1-3 nitrogen heteroatoms.

In some embodiments, the compound is a compound of formula II-a-1, or a pharmaceutically acceptable salt thereof. In some embodiments, when X¹ is CH or N, X² is CH and Ring B is phenyl, then ring A is other than phenyl or a 6-membered monocyclic heteroaryl ring containing 1-3 nitrogen heteroatoms.

In some embodiments, the compound is a compound of formula II-b-1, or a pharmaceutically acceptable salt thereof. In some embodiments, when X¹ is CH or N, X² is CH and Ring B is phenyl, then ring A is other than phenyl or a 6-membered monocyclic heteroaryl ring containing 1-3 nitrogen heteroatoms.

In some embodiments, the compound is a compound of formula II-c-1, or a pharmaceutically acceptable salt thereof. In some embodiments, when X¹ is CH or N, X² is CH and Ring B is phenyl, then ring A is other than phenyl or a 6-membered monocyclic heteroaryl ring containing 1-3 nitrogen heteroatoms.

In certain embodiments, the present invention provides a compound of formula I-1, I-1′, I-2, I-2′, I-3 or I-3′, wherein ERBM is

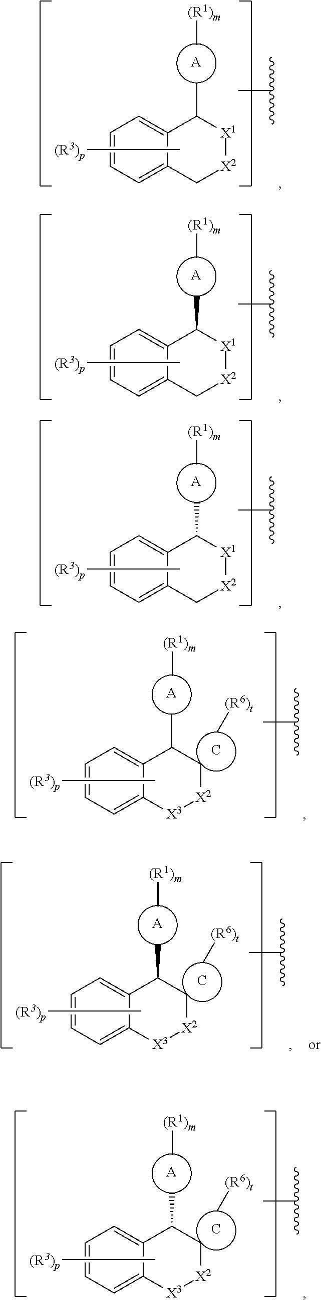

thereby forming a compound of one of formulas VIII-a, VIII-b, VIII-c, IX-a, IX-b or IX-c, respectively:

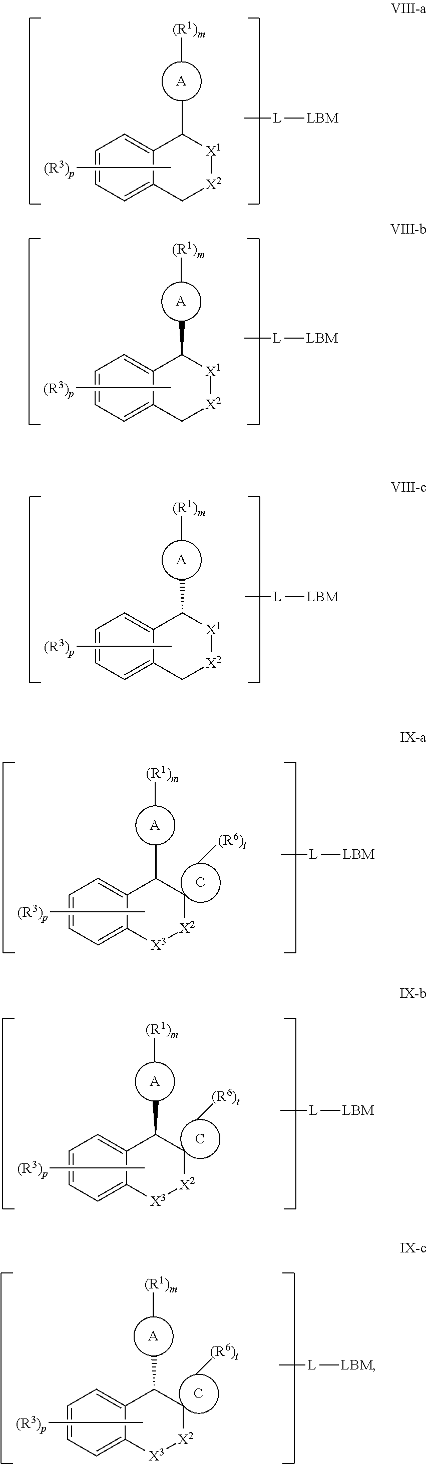

or a pharmaceutically acceptable salt thereof, wherein each of X¹, X², X³, Ring A, Ring C, R¹, R³, R⁶, m, p, t, L, and LBM is as defined in embodiments and classes and subclasses herein.

In some embodiments, the compound is a compound of formula VIII-a, or a pharmaceutically acceptable salt thereof.

In some embodiments, the compound is a compound of formula VIII-b, or a pharmaceutically acceptable salt thereof.

In some embodiments, the compound is a compound of formula VIII-c, or a pharmaceutically acceptable salt thereof.

In some embodiments, the compound is a compound of formula IX-a, or a pharmaceutically acceptable salt thereof.

In some embodiments, the compound is a compound of formula IX-b, or a pharmaceutically acceptable salt thereof.

In some embodiments, the compound is a compound of formula IX-c, or a pharmaceutically acceptable salt thereof.

In some embodiments, the compound is a compound of one of formulas VIII-a-1, VIII-b-1, or VIII-c-1:

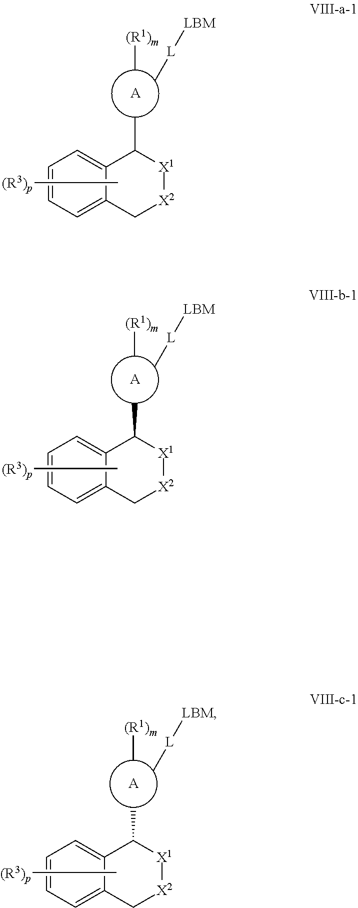

or a pharmaceutically acceptable salt thereof, wherein each of X¹, X², Ring A, R¹, R³, m, p, L, and LBM is as defined in embodiments and classes and subclasses herein.

In some embodiments, the compound is a compound of formula VIII-a-1, or a pharmaceutically acceptable salt thereof.

In some embodiments, the compound is a compound of formula VIII-b-1, or a pharmaceutically acceptable salt thereof.

In some embodiments, the compound is a compound of formula VIII-c-1, or a pharmaceutically acceptable salt thereof.

In some embodiments, the compound is a compound of one of formulas IX-a-1, IX-b-1 or IX-c-1

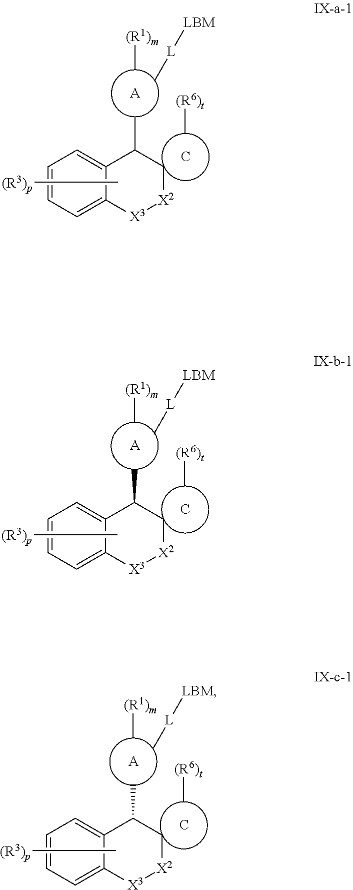

or a pharmaceutically acceptable salt thereof, wherein each of X², X³, Ring A, Ring C, R¹, R³, R⁶, m, p, t, L, and LBM is as defined in embodiments and classes and subclasses herein.

In some embodiments, the compound is a compound of formula IX-a-1, or a pharmaceutically acceptable salt thereof.

In some embodiments, the compound is a compound of formula IX-b-1, or a pharmaceutically acceptable salt thereof.

In some embodiments, the compound is a compound of formula IX-c-1, or a pharmaceutically acceptable salt thereof.

In certain embodiments, the present invention provides a compound of formula I-1, I-1′, I-2, I-2′, I-3 or I-3′, wherein ERBM is

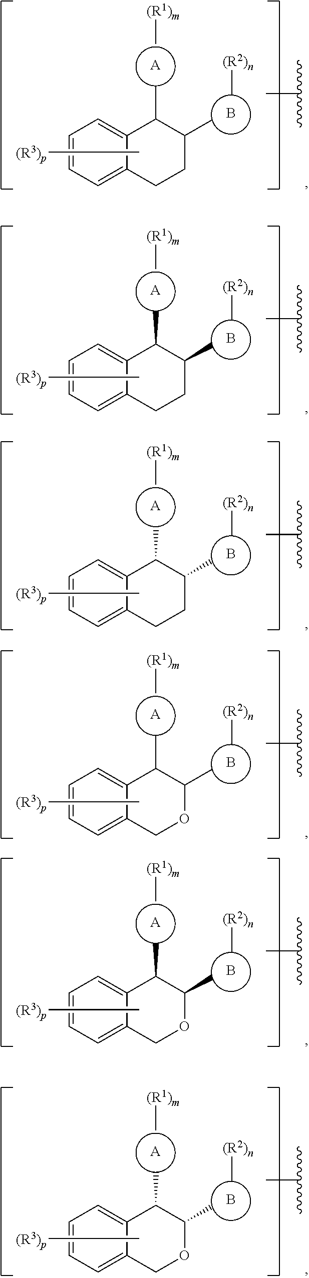

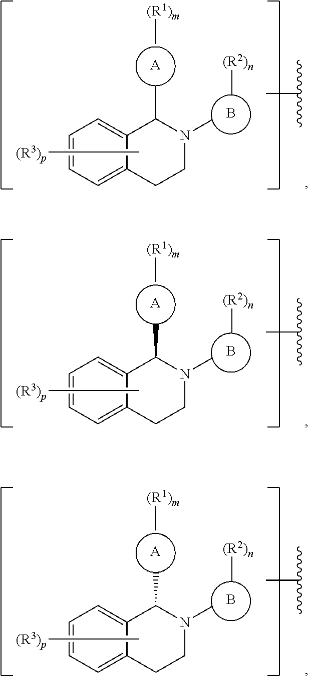

thereby forming a compound of one of formulas II-d, II-e, II-f, II-g, II-h, II-i, II-j, II-k, or II-l, respectively:

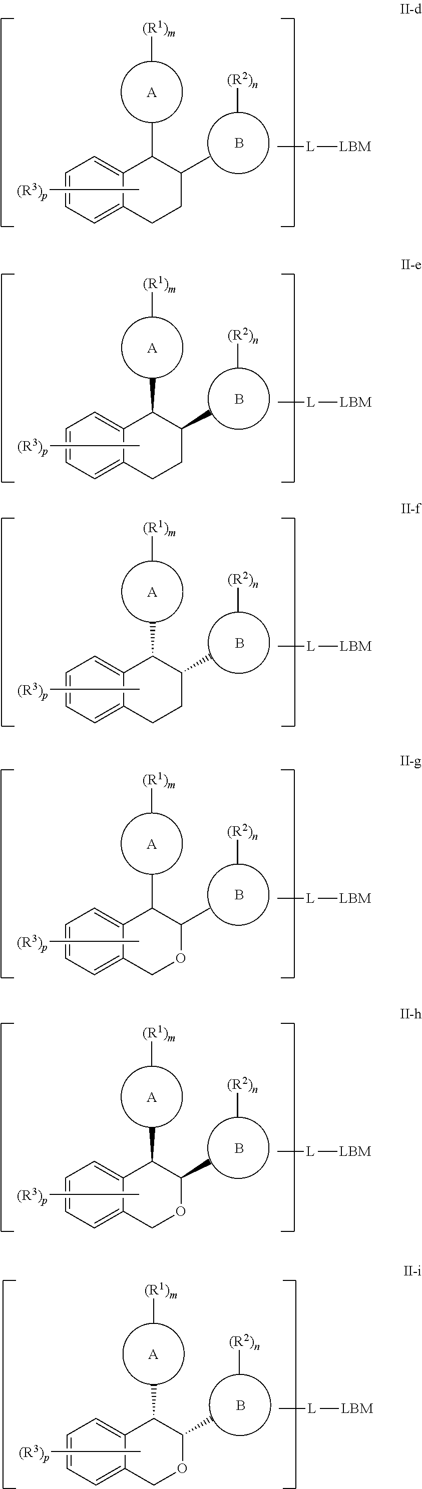

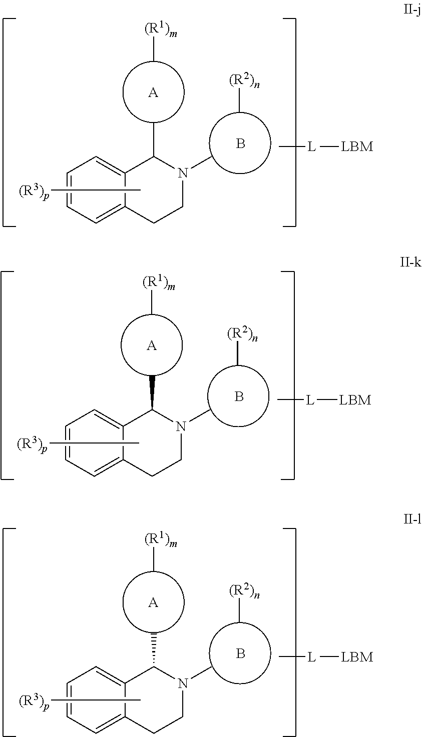

or a pharmaceutically acceptable salt thereof, wherein each of Ring A, Ring B, R¹, R², R³, m, n, p, L, and LBM is as defined in embodiments and classes and subclasses herein.

In some embodiments, when the compound is of formula II-d, II-e, II-f, II-j, II-k or II-l and Ring B is phenyl, then Ring A is other than phenyl or a 6 membered monocyclic heteroaryl ring having 1-3 nitrogen heteroatoms.

In some embodiments, the compound is a compound of formula II-d, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-e, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-f, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-g, or a pharmaceutically acceptable salt thereof.

In some embodiments, the compound is a compound of formula II-h, or a pharmaceutically acceptable salt thereof.

In some embodiments, the compound is a compound of formula II-i, or a pharmaceutically acceptable salt thereof.

In some embodiments, the compound is a compound of formula II-j, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-k, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-l, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In certain embodiments, the present invention provides a compound of formula I-1, I-1′, I-2, I-2′, I-3 or I-3′, wherein ERBM IS

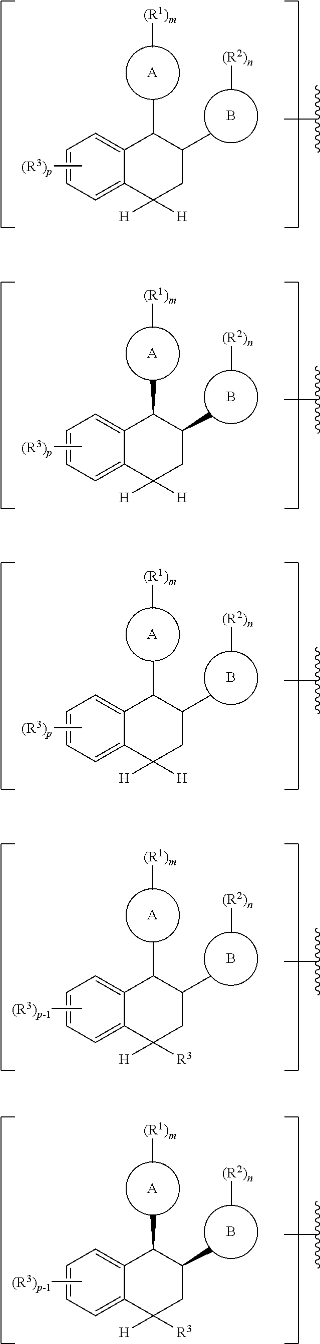

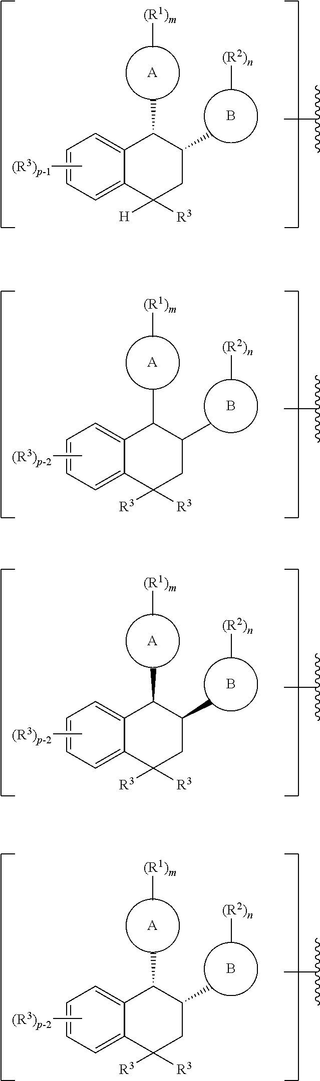

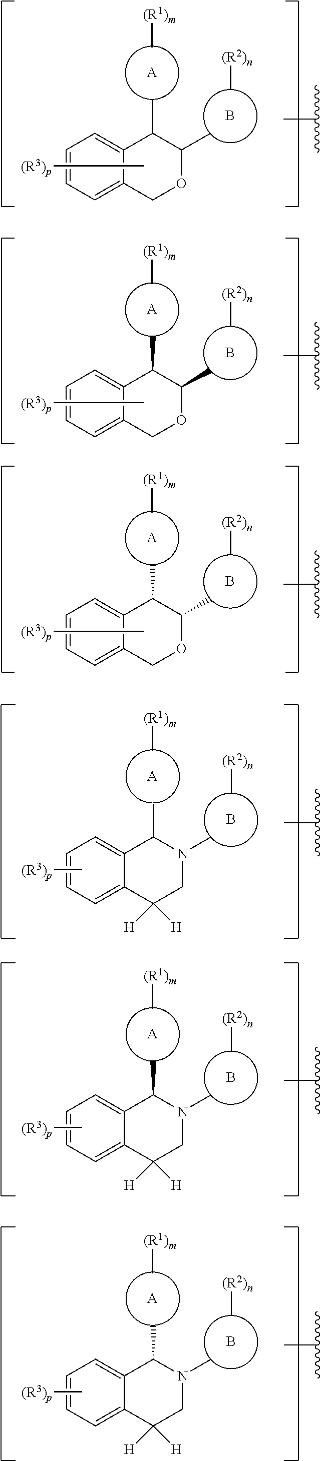

thereby forming a compound of one of formulas II-d-A, II-e-A, II-f-A, II-d-B, II-e-B, II-f-B, II-d-C, II-e-C, II-f-C, II-g, II-h, II-l, II-j-A, II-k-A, or II-l-A, respectively:

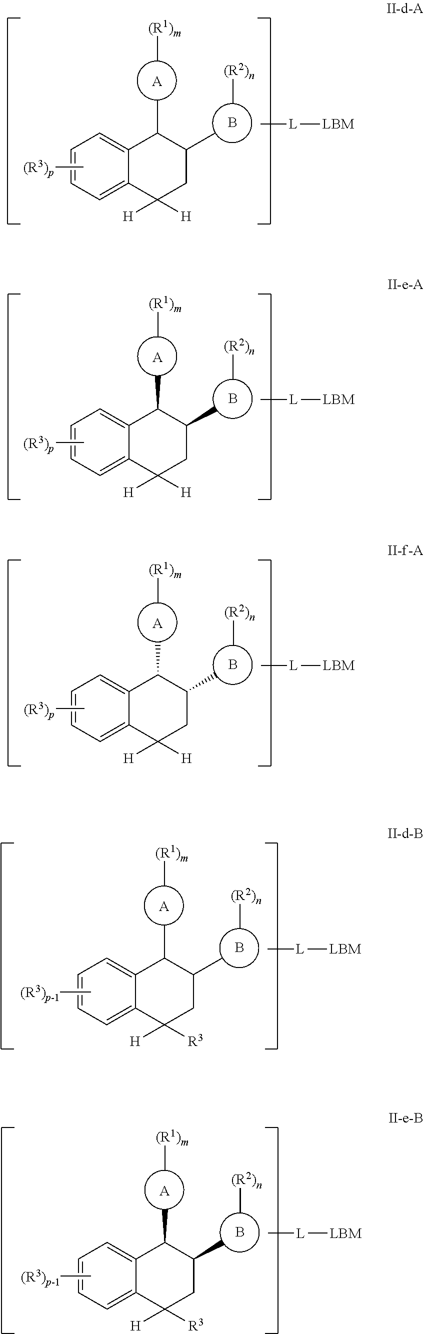

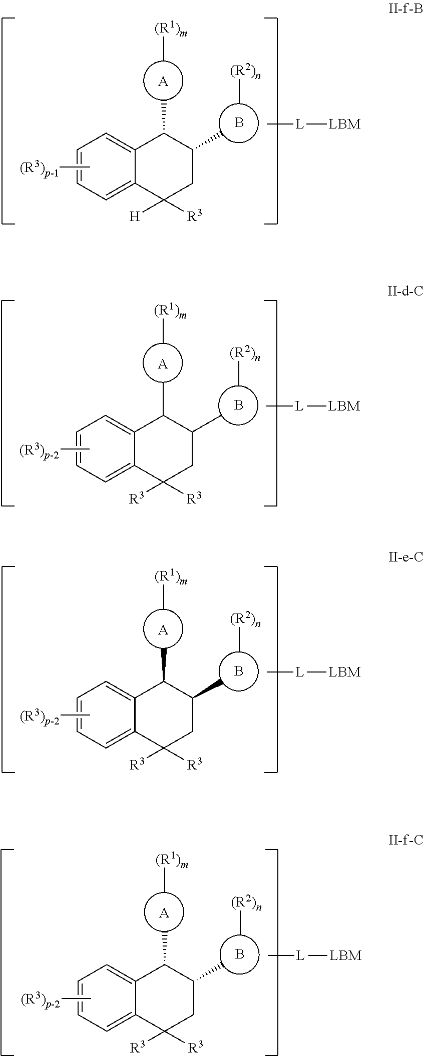

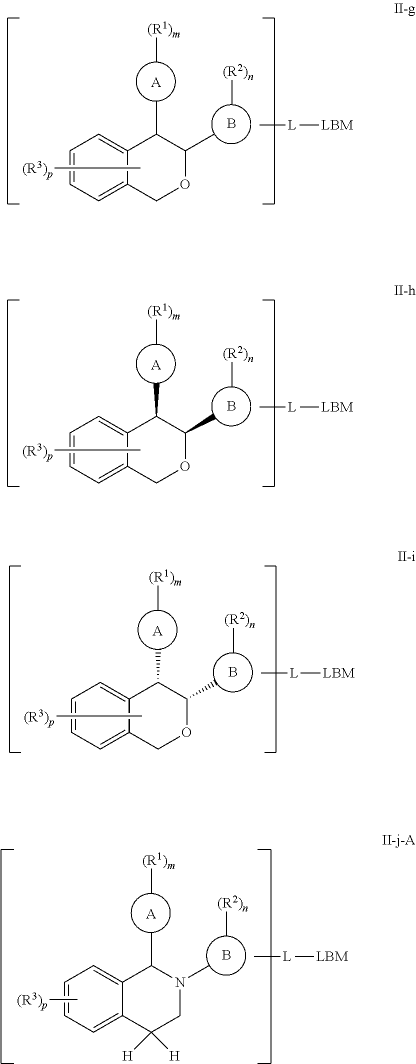

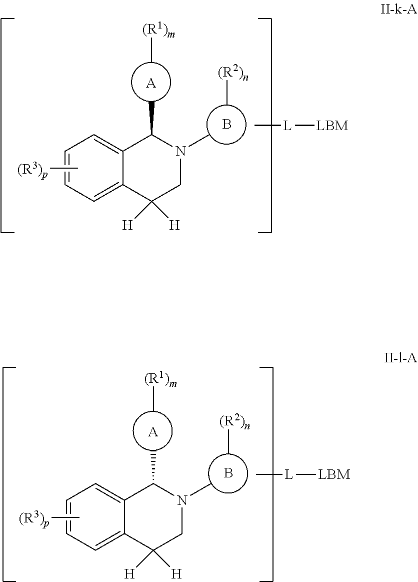

or a pharmaceutically acceptable salt thereof, wherein each of Ring A, Ring B, R¹, R², R³, m, n, p, L, and LBM is as defined in embodiments and classes and subclasses herein.

In some embodiments, when the compound is of formula II-d-A, II-e-A, II-f-A, II-j-A, II-k-A and II-l-A and Ring B is phenyl, then Ring A is other than phenyl or a 6 membered monocyclic heteroaryl ring having 1-3 nitrogen heteroatoms.

In some embodiments, the compound is a compound of formula II-d-A, or a pharmaceutically acceptable salt thereof. In some embodiments, when Ring Bis phenyl, then Ring A is other than phenyl or a 6 membered monocyclic heteroaryl ring having 1-3 nitrogen heteroatoms.

In some embodiments, the compound is a compound of formula II-e-A, or a pharmaceutically acceptable salt thereof. In some embodiments, when Ring Bis phenyl, then Ring A is other than phenyl or a 6 membered monocyclic heteroaryl ring having 1-3 nitrogen heteroatoms.

In some embodiments, the compound is a compound of formula II-f-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-d-B, or a pharmaceutically acceptable salt thereof.

In some embodiments, the compound is a compound of formula II-e-B, or a pharmaceutically acceptable salt thereof.

In some embodiments, the compound is a compound of formula II-f-B, or a pharmaceutically acceptable salt thereof.

In some embodiments, the compound is a compound of formula II-d-C, or a pharmaceutically acceptable salt thereof.

In some embodiments, the compound is a compound of formula II-e-C, or a pharmaceutically acceptable salt thereof.

In some embodiments, the compound is a compound of formula II-f-C, or a pharmaceutically acceptable salt thereof.

In some embodiments, the compound is a compound of formula II-j-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-k-A, or a pharmaceutically acceptable salt thereof. In some embodiments, when Ring Bis phenyl, then Ring A is other than phenyl or a 6 membered monocyclic heteroaryl ring having 1-3 nitrogen heteroatoms.

In some embodiments, the compound is a compound of formula II-l-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-j-B, or a pharmaceutically acceptable salt thereof.

In some embodiments, the compound is a compound of formula II-k-B, or a pharmaceutically acceptable salt thereof.

In some embodiments, the compound is a compound of formula II-l-B, or a pharmaceutically acceptable salt thereof.

In some embodiments, the compound is a compound of formula II-j-C, or a pharmaceutically acceptable salt thereof.

In some embodiments, the compound is a compound of formula II-k-C, or a pharmaceutically acceptable salt thereof.

In some embodiments, the compound is a compound of formula II-l-C, or a pharmaceutically acceptable salt thereof.

In some embodiments, the compound is a compound of one of formulas II-d-1, II-e-1, II-f-1, II-g-1, II-h-1, II-i-1, II-j-1, II-k-1, or II-1-1:

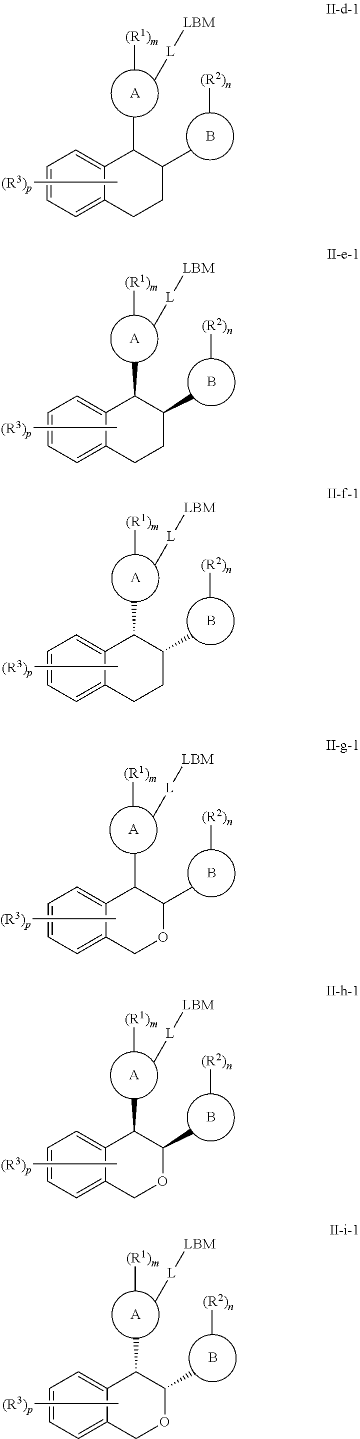

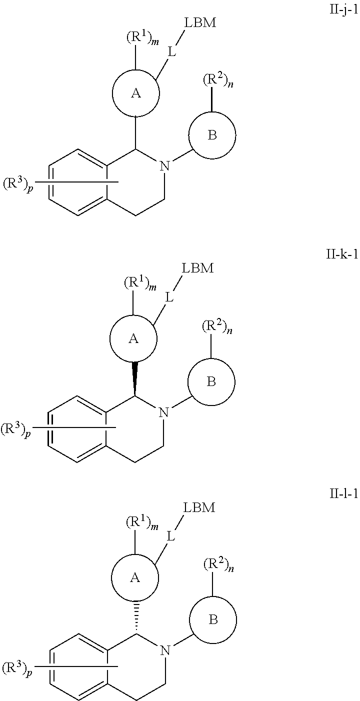

or a pharmaceutically acceptable salt thereof, wherein each of Ring A, Ring B, R¹, R², R³, m, n, p, L, and LBM is as defined in embodiments and classes and subclasses herein.

In some embodiments, when the compound is of formula II-d-1, II-e-1, II-f-1, II-j-1, II-k-1 or II-1-1 and Ring B is phenyl, then Ring A is other than phenyl or a 6 membered monocyclic heteroaryl ring having 1-3 nitrogen heteroatoms.

In some embodiments, the compound is a compound of formula II-d-1, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-e-1, or a pharmaceutically acceptable salt thereof. In some embodiments, when Ring Bis phenyl, then Ring A is other than phenyl or a 6 membered monocyclic heteroaryl ring having 1-3 nitrogen heteroatoms.

In some embodiments, the compound is a compound of formula II-f-1, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-g-1, or a pharmaceutically acceptable salt thereof.

In some embodiments, the compound is a compound of formula II-h-1, or a pharmaceutically acceptable salt thereof.

In some embodiments, the compound is a compound of formula II-i-1, or a pharmaceutically acceptable salt thereof.

In some embodiments, the compound is a compound of formula II-j-1, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-k-1, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-1-1, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-d-1-A, II-e-1-A, II-f-1-A. II-d-1-B, II-e-1-B, II-f-1-B, II-d-1-C, II-e-1-C, II-f-1-C, II-j-1-A, II-k-1-A, or II-l-1-A:

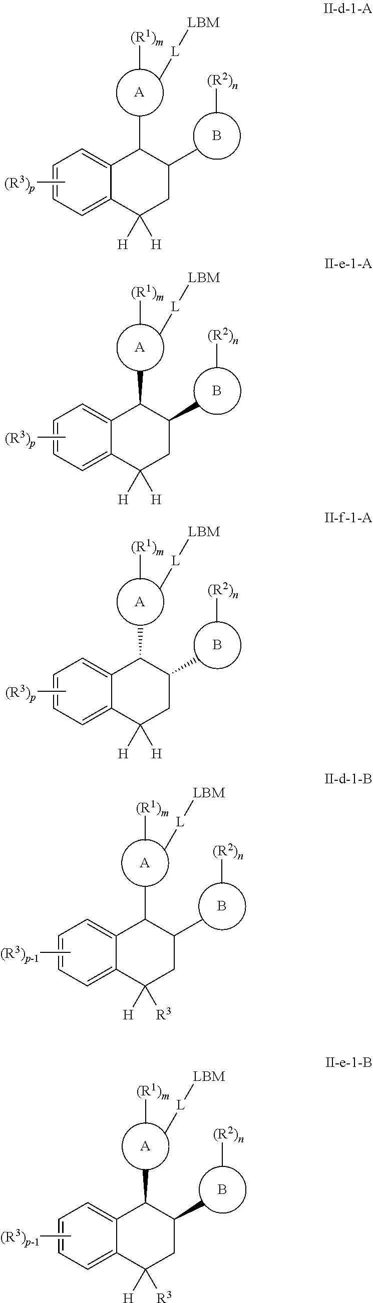

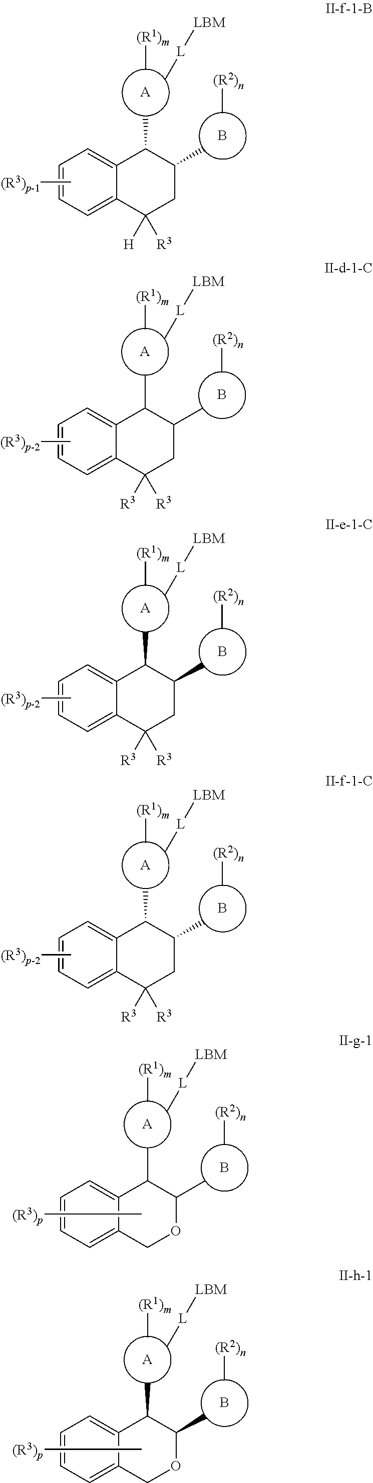

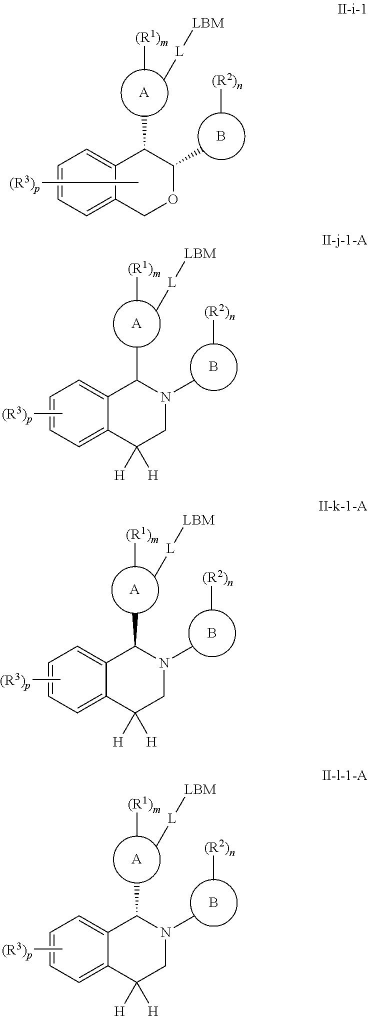

or a pharmaceutically acceptable salt thereof, wherein each of Ring A, Ring B, R¹, R², R³, m, n, p, L, and LBM is as defined in embodiments and classes and subclasses herein.

In some embodiments, when the compound is of formula II-d-1-A, II-e-1-A, II-f-1-A, II-j-1-A, II-k-1-A and II-l-1-A and Ring B is phenyl, then Ring A is other than phenyl or a 6 membered monocyclic heteroaryl ring having 1-3 nitrogen heteroatoms.

In some embodiments, the compound is a compound of formula II-d-A, or a pharmaceutically acceptable salt thereof. In some embodiments, when Ring Bis phenyl, then Ring A is other than phenyl or a 6 membered monocyclic heteroaryl ring having 1-3 nitrogen heteroatoms.

In some embodiments, the compound is a compound of formula II-e-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-f-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-d-B, or a pharmaceutically acceptable salt thereof.

In some embodiments, the compound is a compound of formula II-e-B, or a pharmaceutically acceptable salt thereof.

In some embodiments, the compound is a compound of formula II-f-B, or a pharmaceutically acceptable salt thereof.

In some embodiments, the compound is a compound of formula II-d-C, or a pharmaceutically acceptable salt thereof.

In some embodiments, the compound is a compound of formula II-e-C, or a pharmaceutically acceptable salt thereof.

In some embodiments, the compound is a compound of formula II-f-C, or a pharmaceutically acceptable salt thereof.

In some embodiments, the compound is a compound of formula II-j-A, or a pharmaceutically acceptable salt thereof. In some embodiments, when Ring Bis phenyl, then Ring A is other than phenyl or a 6 membered monocyclic heteroaryl ring having 1-3 nitrogen heteroatoms.

In some embodiments, the compound is a compound of formula II-k-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-l-A, or a pharmaceutically acceptable salt thereof. In some embodiments, when Ring B is phenyl, then Ring A is other than phenyl or a 6 membered monocyclic heteroaryl ring having 1-3 nitrogen heteroatoms.

In some embodiments, the compound is a compound of formula II-j-B, or a pharmaceutically acceptable salt thereof.

In some embodiments, the compound is a compound of formula II-k-B, or a pharmaceutically acceptable salt thereof.

In some embodiments, the compound is a compound of formula II-l-B, or a pharmaceutically acceptable salt thereof.

In some embodiments, the compound is a compound of formula II-j-C, or a pharmaceutically acceptable salt thereof.

In some embodiments, the compound is a compound of formula II-k-C, or a pharmaceutically acceptable salt thereof.

In some embodiments, the compound is a compound of formula II-l-C, or a pharmaceutically acceptable salt thereof.

In some embodiments, the compound is a compound of formula II-m-1:

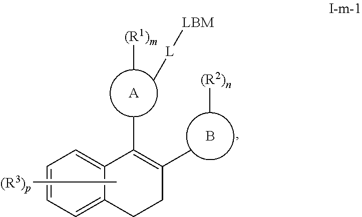

or a pharmaceutically acceptable salt thereof.

In certain embodiments, the present invention provides a compound of formula I-1, I-1′, I-2, I-2′, I-3 or I-3′, wherein ERBM is

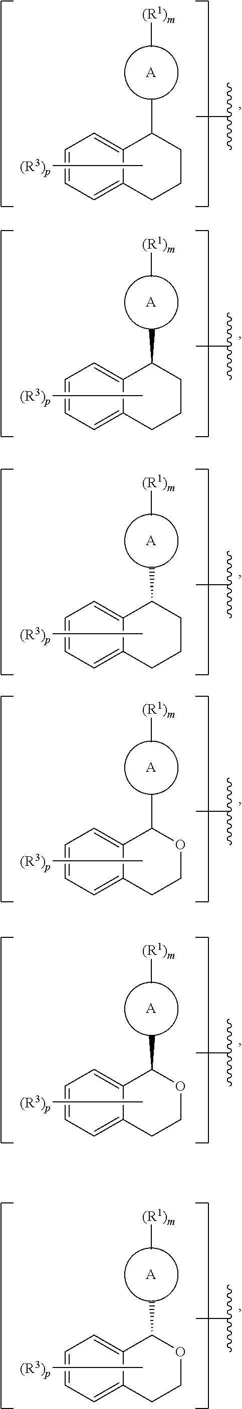

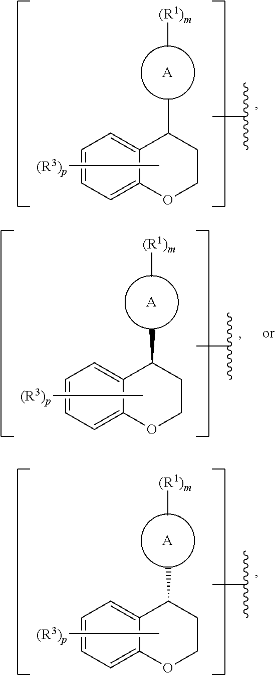

thereby forming a compound of one of formulas II-n, II-o, II-p, II-q, II-r, II-s, II-t, II-u or II-v, respectively:

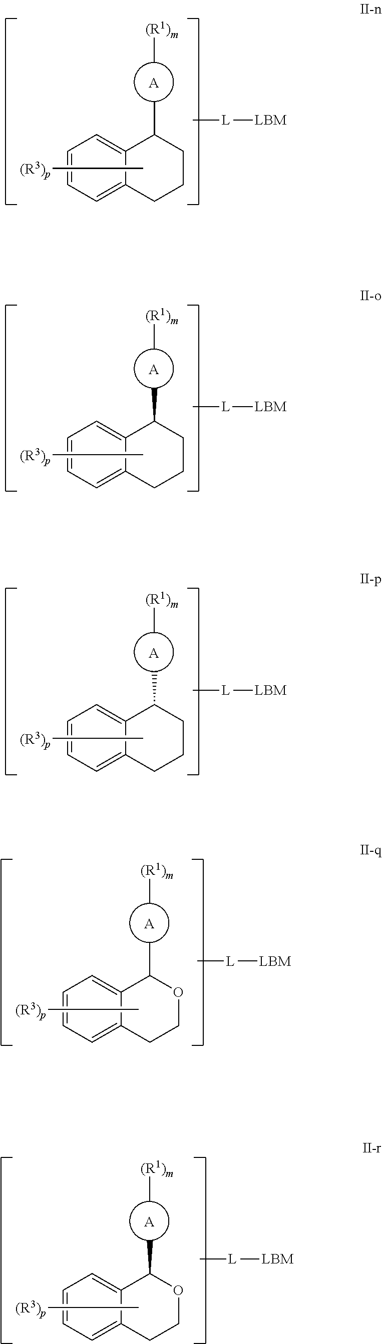

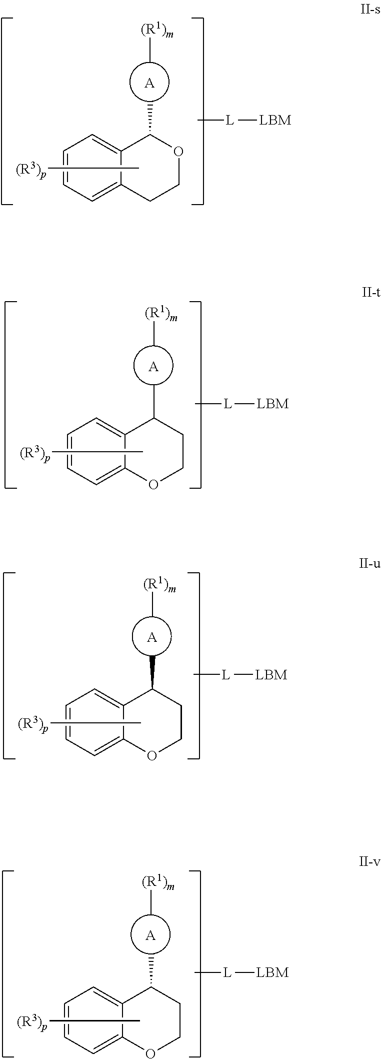

or a pharmaceutically acceptable salt thereof, wherein each of Ring A, R¹, R³, m, p, L, and LBM is as defined in embodiments and classes and subclasses herein.

In some embodiments, the compound is a compound of formula II-n, or a pharmaceutically acceptable salt thereof.

In some embodiments, the compound is a compound of formula II-o, or a pharmaceutically acceptable salt thereof.

In some embodiments, the compound is a compound of formula II-p, or a pharmaceutically acceptable salt thereof.

In some embodiments, the compound is a compound of formula II-q, or a pharmaceutically acceptable salt thereof.

In some embodiments, the compound is a compound of formula II-r, or a pharmaceutically acceptable salt thereof.

In some embodiments, the compound is a compound of formula II-s, or a pharmaceutically acceptable salt thereof.

In some embodiments, the compound is a compound of formula II-t, or a pharmaceutically acceptable salt thereof.

In some embodiments, the compound is a compound of formula II-u, or a pharmaceutically acceptable salt thereof.

In some embodiments, the compound is a compound of formula II-v, or a pharmaceutically acceptable salt thereof.

In certain embodiments, the present invention provides a compound of formula I-1, I-1′, I-2, I-2′, I-3 or I-3′, wherein ERBM is

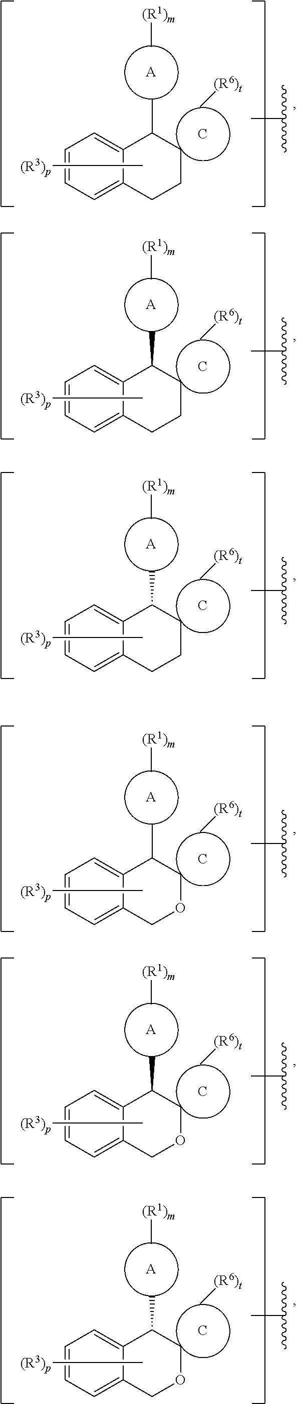

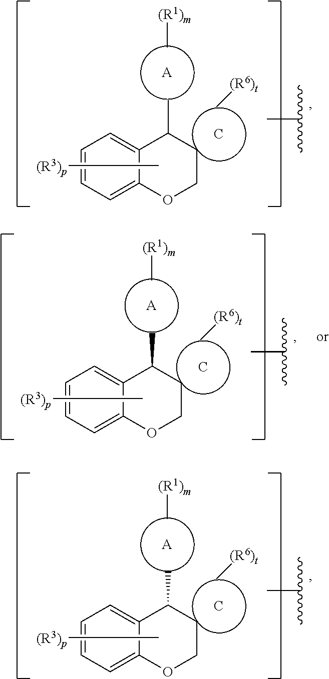

thereby forming a compound of one of formulas II-w, II-x, II-y, II-z, II-aa, II-bb, II-cc, II-dd or II-ee, respectively:

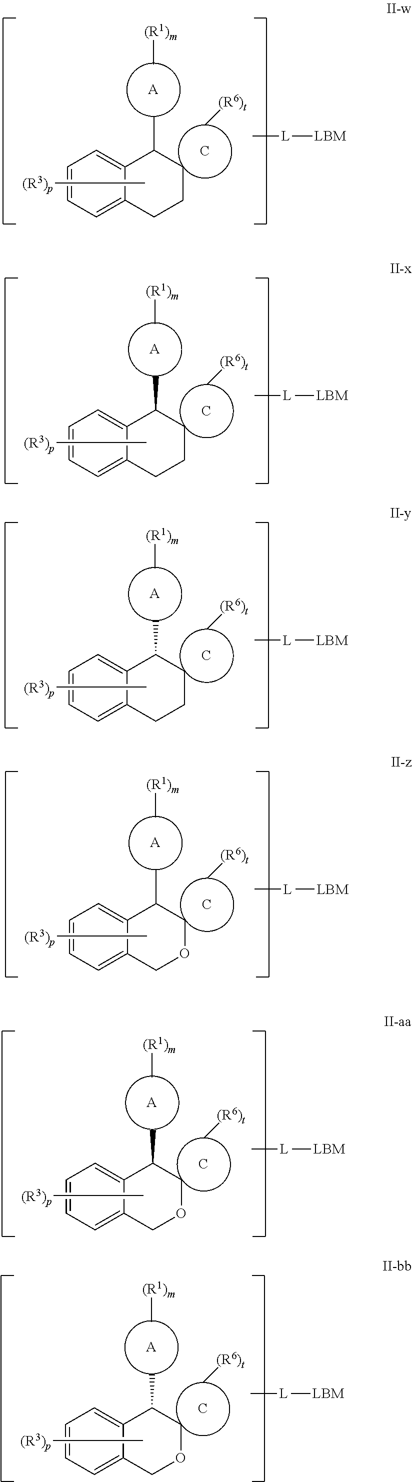

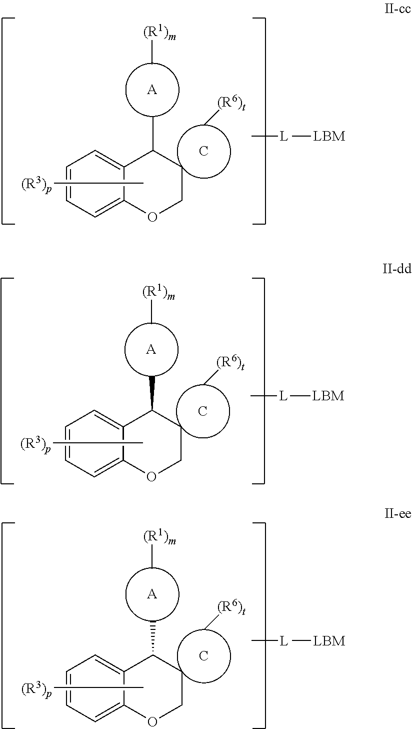

or a pharmaceutically acceptable salt thereof, wherein each of Ring A, Ring C, R¹, R³, R⁶, m, p, t, L, and LBM is as defined in embodiments and classes and subclasses herein.

In some embodiments, the compound is a compound of formula II-w, or a pharmaceutically acceptable salt thereof.

In some embodiments, the compound is a compound of formula II-x, or a pharmaceutically acceptable salt thereof.

In some embodiments, the compound is a compound of formula II-y, or a pharmaceutically acceptable salt thereof.

In some embodiments, the compound is a compound of formula II-z, or a pharmaceutically acceptable salt thereof.

In some embodiments, the compound is a compound of formula II-aa, or a pharmaceutically acceptable salt thereof.

In some embodiments, the compound is a compound of formula II-bb, or a pharmaceutically acceptable salt thereof.

In some embodiments, the compound is a compound of formula II-cc, or a pharmaceutically acceptable salt thereof.

In some embodiments, the compound is a compound of formula II-dd, or a pharmaceutically acceptable salt thereof.

In some embodiments, the compound is a compound of formula II-ee, or a pharmaceutically acceptable salt thereof.

In some embodiments, the compound is a compound of one of formulas II-n-1, II-o-1, II-p-1, II-q-1, II-r-1, II-s-1, II-t-1, II-u-1 or II-v-1:

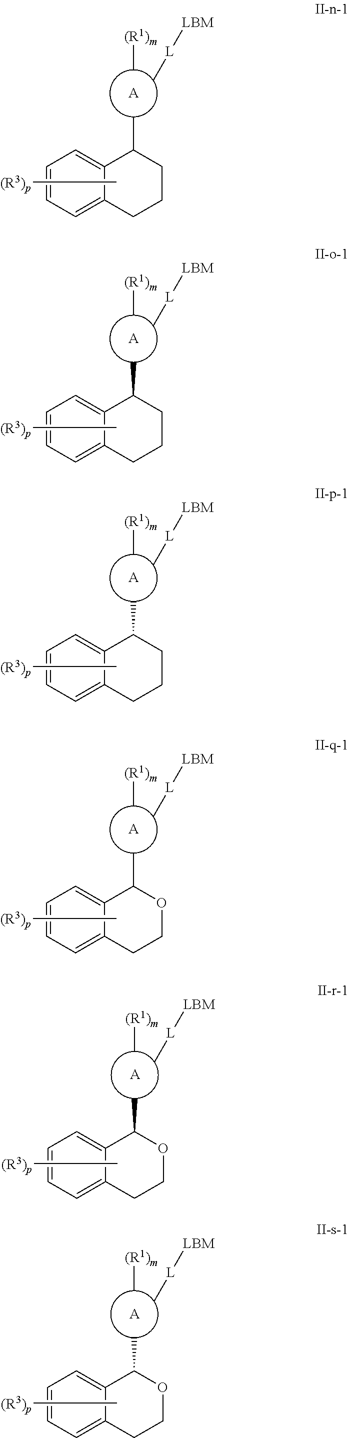

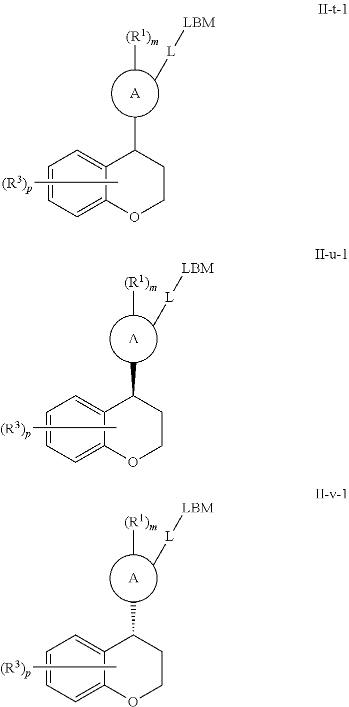

or a pharmaceutically acceptable salt thereof, wherein each of Ring A, R¹, R³, m, p, L, and LBM is as defined in embodiments and classes and subclasses herein.

In some embodiments, the compound is a compound of formula II-n-1, or a pharmaceutically acceptable salt thereof.

In some embodiments, the compound is a compound of formula II-o-1, or a pharmaceutically acceptable salt thereof.

In some embodiments, the compound is a compound of formula II-p-1, or a pharmaceutically acceptable salt thereof.

In some embodiments, the compound is a compound of formula II-q-1, or a pharmaceutically acceptable salt thereof.

In some embodiments, the compound is a compound of formula II-r-1, or a pharmaceutically acceptable salt thereof.

In some embodiments, the compound is a compound of formula II-s-1, or a pharmaceutically acceptable salt thereof.

In some embodiments, the compound is a compound of formula II-t-1, or a pharmaceutically acceptable salt thereof.

In some embodiments, the compound is a compound of formula II-u-1 or a pharmaceutically acceptable salt thereof.

In some embodiments, the compound is a compound of formula II-v-1 or a pharmaceutically acceptable salt thereof.

In some embodiments, the compound is a compound of one of formulas II-w-1, II-x-1, II-y-1, II-z-1, II-aa-1, II-bb-1, II-cc-1, II-dd-1 or II-ee-1:

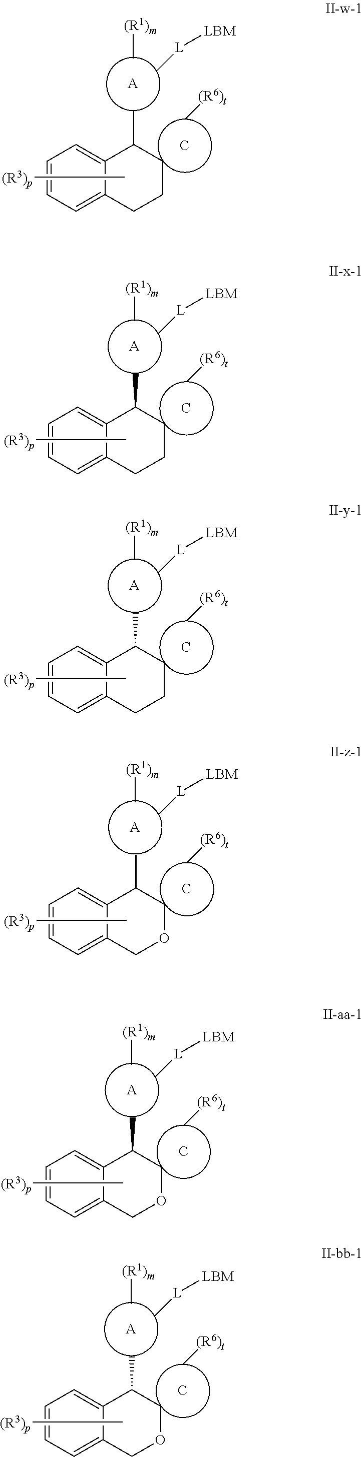

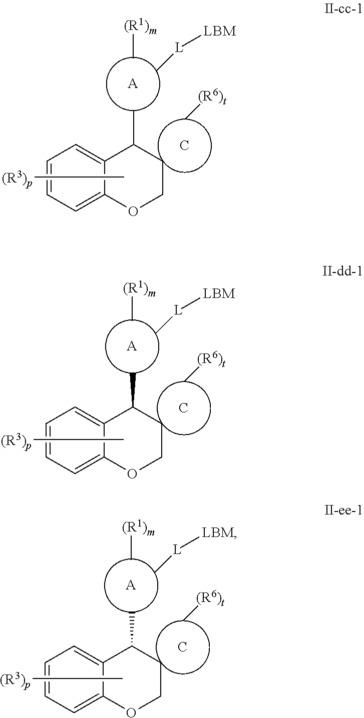

or a pharmaceutically acceptable salt thereof, wherein each of Ring A, Ring C, R¹, R³, R⁶, m, p, t, L, and LBM is as defined in embodiments and classes and subclasses herein.

In some embodiments, the compound is a compound of formula II-w-1, or a pharmaceutically acceptable salt thereof.

In some embodiments, the compound is a compound of formula II-x-1, or a pharmaceutically acceptable salt thereof.

In some embodiments, the compound is a compound of formula II-y-1, or a pharmaceutically acceptable salt thereof.

In some embodiments, the compound is a compound of formula II-z-1, or a pharmaceutically acceptable salt thereof.

In some embodiments, the compound is a compound of formula II-aa-1, or a pharmaceutically acceptable salt thereof.

In some embodiments, the compound is a compound of formula II-bb-1, or a pharmaceutically acceptable salt thereof.

In some embodiments, the compound is a compound of formula II-cc-1, or a pharmaceutically acceptable salt thereof.

In some embodiments, the compound is a compound of formula II-dd-1, or a pharmaceutically acceptable salt thereof.

In some embodiments, the compound is a compound of formula II-ee-1, or a pharmaceutically acceptable salt thereof.

In some embodiments, the compound is a compound of one of formulas II-ff-1, II-gg-1, II-hh-1, II-ii-1, II-jj-1, II-kk-1, II-ll-1, II-mm-1 or II-nn-1:

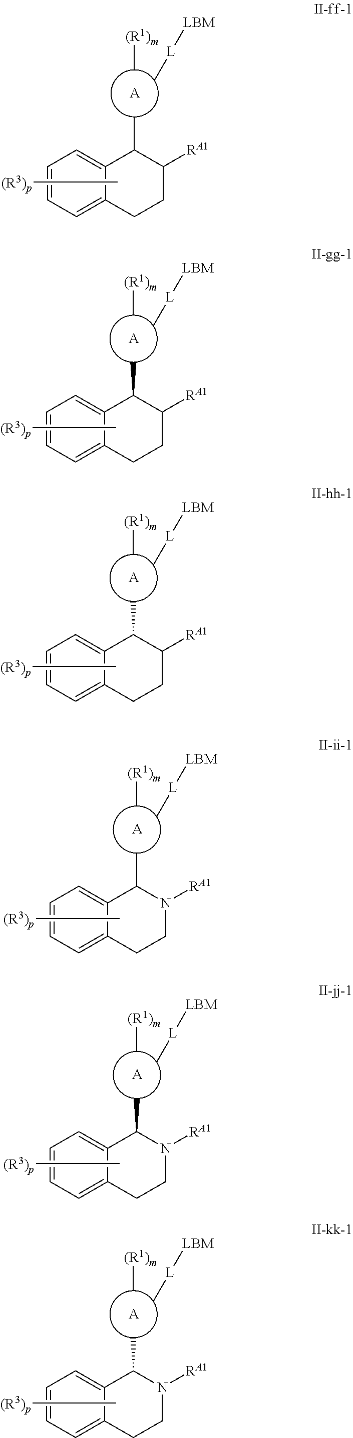

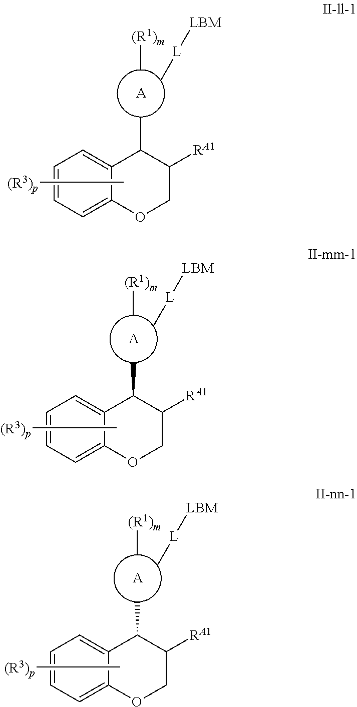

or a pharmaceutically acceptable salt thereof, wherein each of Ring A, R^{A1}, R¹, R³, m, p, L, and LBM is as defined in embodiments and classes and subclasses herein.

In some embodiments, the compound is a compound of formula II-ff-1, or a pharmaceutically acceptable salt thereof.

In some embodiments, the compound is a compound of formula II-gg-1, or a pharmaceutically acceptable salt thereof.

In some embodiments, the compound is a compound of formula II-hh-1, or a pharmaceutically acceptable salt thereof.

In some embodiments, the compound is a compound of formula II-ii-1, or a pharmaceutically acceptable salt thereof.

In some embodiments, the compound is a compound of formula II-jj-1, or a pharmaceutically acceptable salt thereof.

In some embodiments, the compound is a compound of formula II-kk-1, or a pharmaceutically acceptable salt thereof.

In some embodiments, the compound is a compound of formula II-ll-1, or a pharmaceutically acceptable salt thereof.

In some embodiments, the compound is a compound of formula II-mm-1, or a pharmaceutically acceptable salt thereof.

In some embodiments, the compound is a compound of formula II-nn-1, or a pharmaceutically acceptable salt thereof.

In certain embodiments, the present invention provides a compound of formula I-1, I-1′, I-2, I-2′, I-3 or I-3′, wherein ERBM is

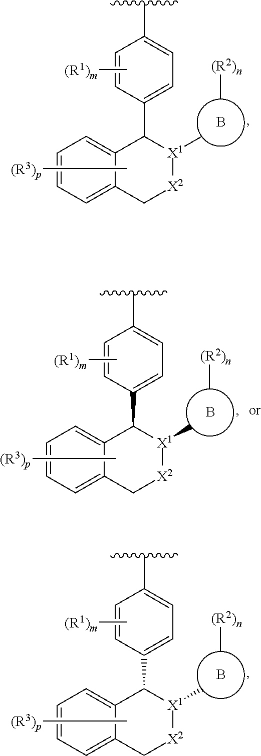

thereby forming a compound of one of formulas III-a, III-b, or III-c, respectively:

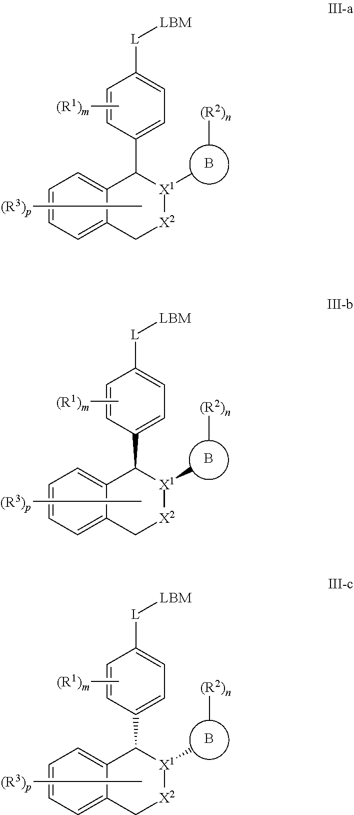

or a pharmaceutically acceptable salt thereof, wherein each of Ring B. R¹, R², R³, X¹, X², m, n, p, L, and LBM is as defined in embodiments and classes and subclasses herein.

In some embodiments of formulas III-a, III-b and III-c, when X¹ is CH or N and X² is CH₂, then Ring B is other than phenyl.

In some embodiments, the compound is a compound of formula III-a, or a pharmaceutically acceptable salt thereof. In some embodiments, when X¹ is CH or N and X² is CH₂, then Ring B is other than phenyl.

In some embodiments, the compound is a compound of formula III-b, or a pharmaceutically acceptable salt thereof. In some embodiments, when X¹ is CH or N and X² is CH₂, then Ring B is other than phenyl.

In some embodiments, the compound is a compound of formula III-c, or a pharmaceutically acceptable salt thereof. In some embodiments, when X¹ is CH or N and X² is CH₂, then Ring B is other than phenyl.

In certain embodiments, the present invention provides a compound of formula I-1, I-1′, I-2, I-2′, I-3 or I-3′, wherein ERBM is

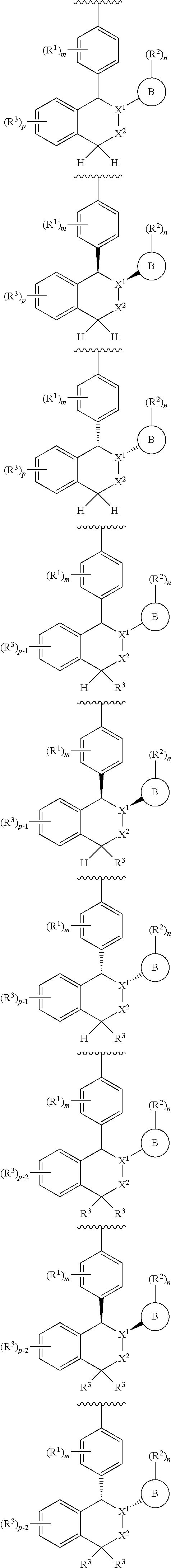

thereby forming a compound of one of formulas III-a-A, III-b-A, III-c-A, III-a-B, III-b-B, III-c-B, III-a-C, III-b-C and III-c-C, respectively:

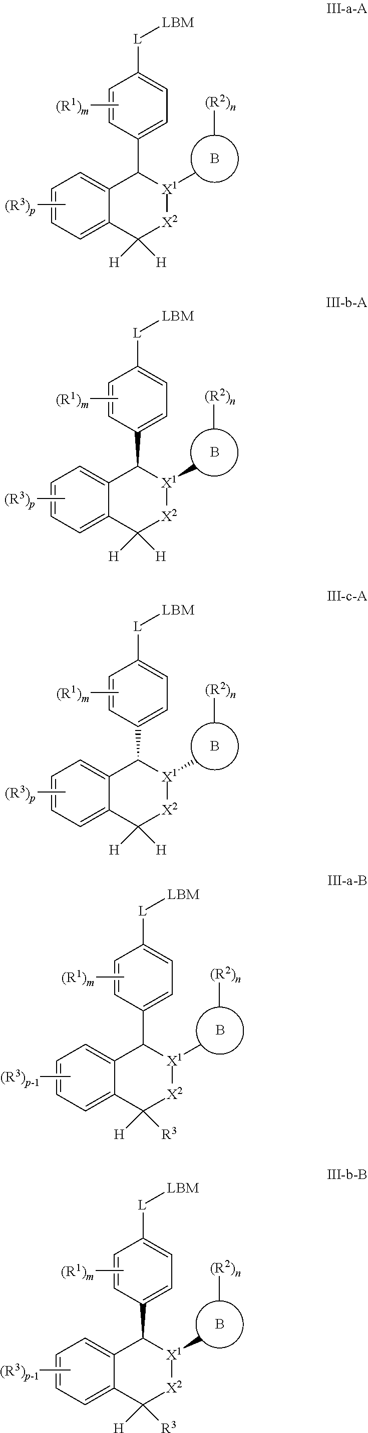

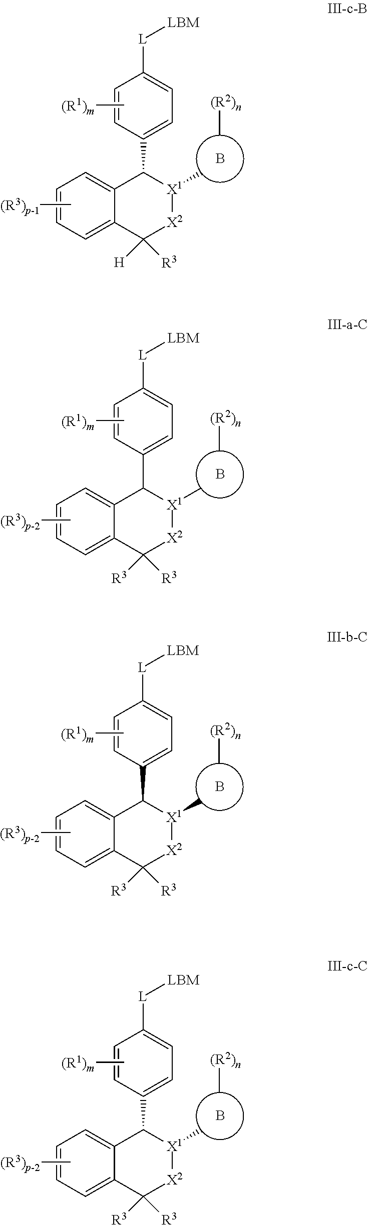

or a pharmaceutically acceptable salt thereof, wherein each of Ring B. R¹, R², R³, X¹, X², m, n, p, L, and LBM is as defined in embodiments and classes and subclasses herein.

In some embodiments of formulas III-a-A, III-b-A and III-c-A, when X¹ is CH or N and X² is CH₂, then Ring B is other than phenyl.

In some embodiments, the compound is a compound of formula III-a-A, or a pharmaceutically acceptable salt thereof. In some embodiments, when X¹ is CH or N and X² is CH₂, then Ring B is other than phenyl.

In some embodiments, the compound is a compound of formula III-b-A, or a pharmaceutically acceptable salt thereof. In some embodiments, when X¹ is CH or N and X² is CH₂, then Ring B is other than phenyl.

In some embodiments, the compound is a compound of formula III-c-A, or a pharmaceutically acceptable salt thereof. In some embodiments, when X¹ is CH or N and X² is CH₂, then Ring B is other than phenyl.

In some embodiments, the compound is a compound of formula III-a-B, or a pharmaceutically acceptable salt thereof.

In some embodiments, the compound is a compound of formula III-b-B, or a pharmaceutically acceptable salt thereof.

In some embodiments, the compound is a compound of formula III-c-B, or a pharmaceutically acceptable salt thereof.

In some embodiments, the compound is a compound of formula III-a-C, or a pharmaceutically acceptable salt thereof.

In some embodiments, the compound is a compound of formula III-b-C, or a pharmaceutically acceptable salt thereof.

In some embodiments, the compound is a compound of formula III-c-C, or a pharmaceutically acceptable salt thereof.

In certain embodiments, the present invention provides a compound of formula I-1, I-1′, I-2, I-2′, I-3 or I-3′, wherein ERBM is

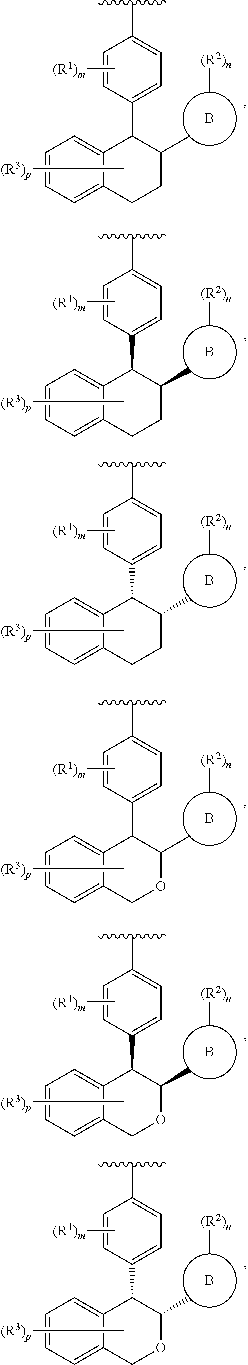

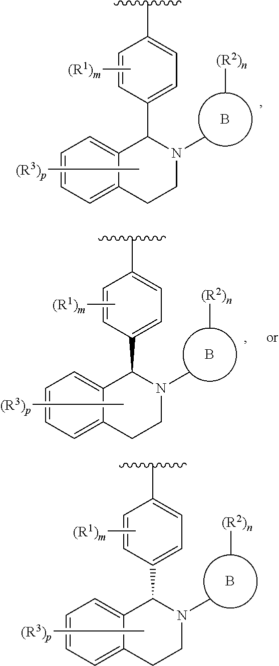

thereby forming a compound of one of formulas III-d, III-e, III-f, III-g, III-h, III-i, III-j, III-k, or III-l, respectively:

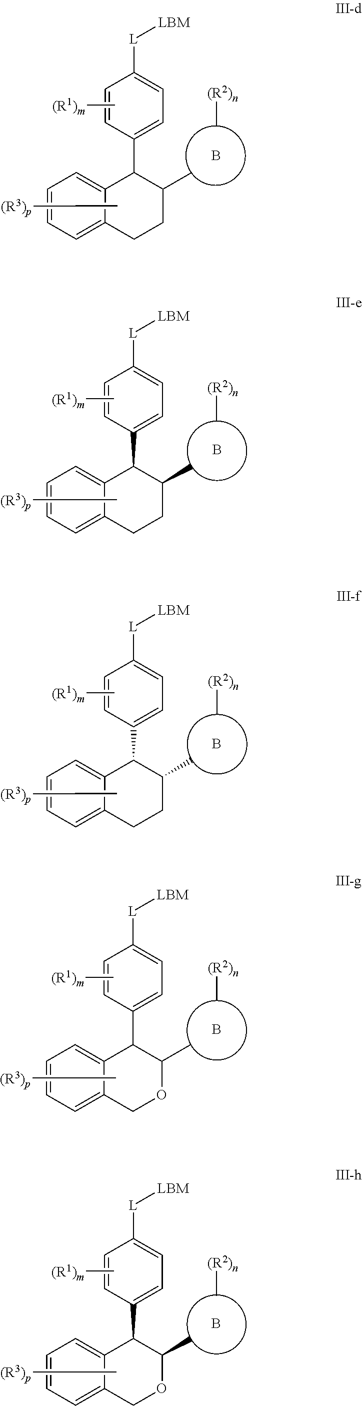

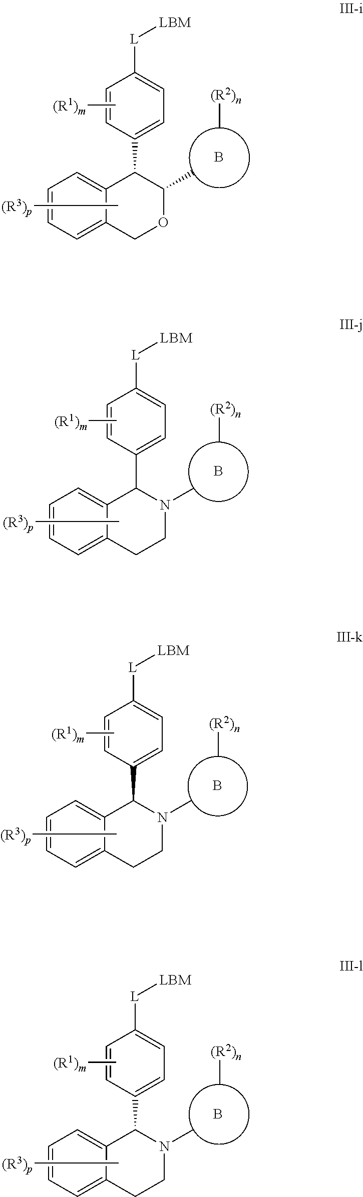

or a pharmaceutically acceptable salt thereof, wherein each of Ring B, R¹, R², R³, m, n, p, L, and LBM is as defined in embodiments and classes and subclasses herein.

In some embodiments, when the compound is of formula III-d, III-e, III-f, III-j, III-k or III-l then Ring B is other than phenyl.

In some embodiments, the compound is a compound of formula III-d, or a pharmaceutically acceptable salt thereof. In some embodiments, Ring B is other than phenyl.

In some embodiments, the compound is a compound of formula III-e, or a pharmaceutically acceptable salt thereof. In some embodiments, Ring B is other than phenyl.

In some embodiments, the compound is a compound of formula III-f, or a pharmaceutically acceptable salt thereof. In some embodiments, Ring B is other than phenyl.

In some embodiments, the compound is a compound of formula III-g, or a pharmaceutically acceptable salt thereof.

In some embodiments, the compound is a compound of formula III-h, or a pharmaceutically acceptable salt thereof.

In some embodiments, the compound is a compound of formula III-i, or a pharmaceutically acceptable salt thereof.

In some embodiments, the compound is a compound of formula III-j, or a pharmaceutically acceptable salt thereof. In some embodiments, Ring B is other than phenyl.

In some embodiments, the compound is a compound of formula III-k, or a pharmaceutically acceptable salt thereof. In some embodiments, Ring B is other than phenyl.

In some embodiments, the compound is a compound of formula III-l, or a pharmaceutically acceptable salt thereof. In some embodiments, Ring B is other than phenyl.

In certain embodiments, the present invention provides a compound of formula I-1, I-1′, I-2, I-2′, I-3 or I-3′, wherein ERBM is

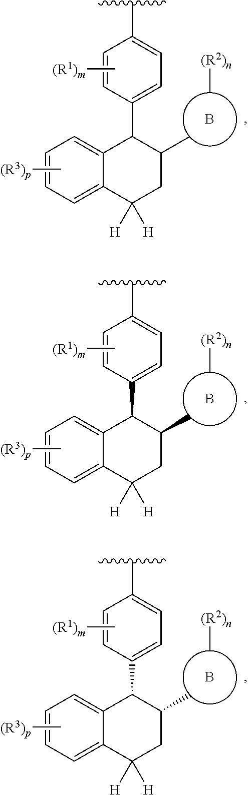

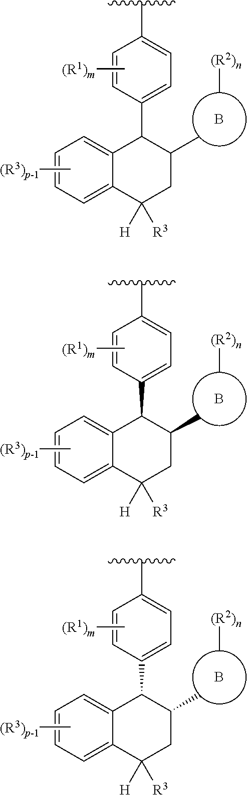

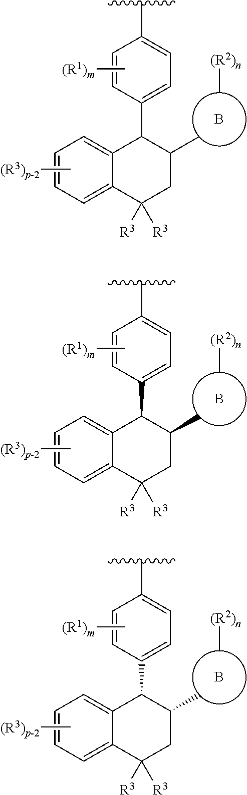

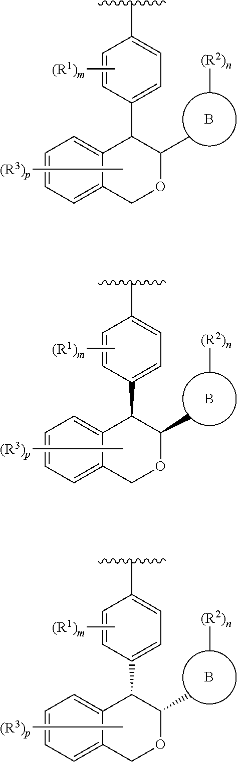

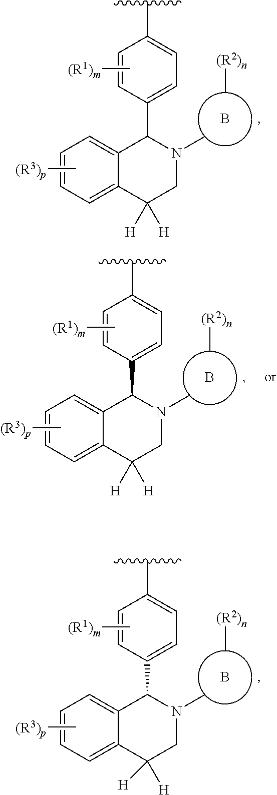

thereby forming a compound of one of formulas III-d-A, III-e-A, III-f-A, III-d-B, III-e-B, III-f-B, III-d-C, III-e-C, III-f-C, III-g, III-h, III-i, III-j-A, III-k-A, or III-l-A, respectively:

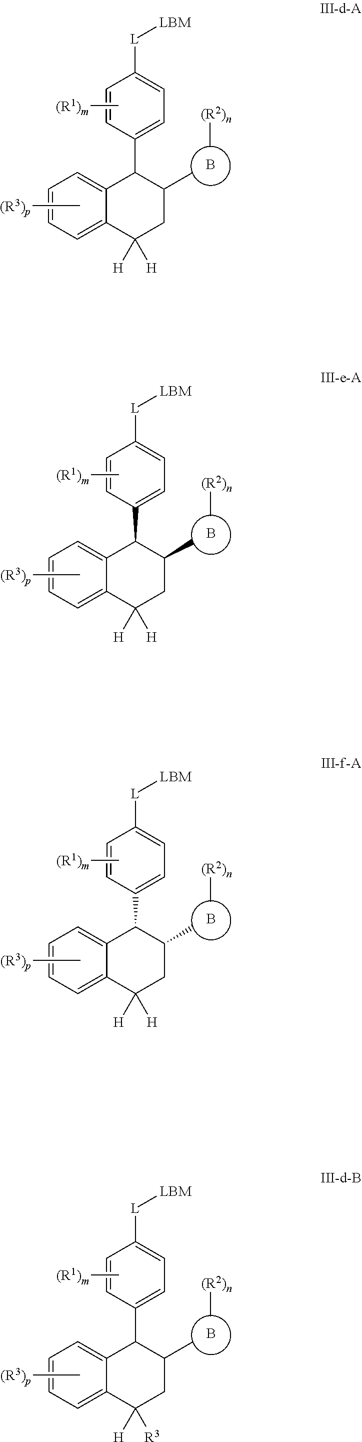

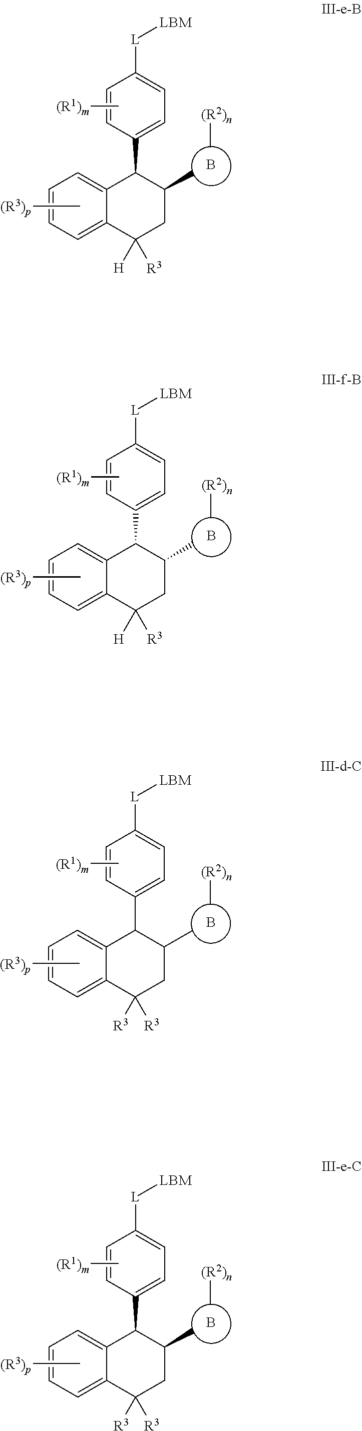

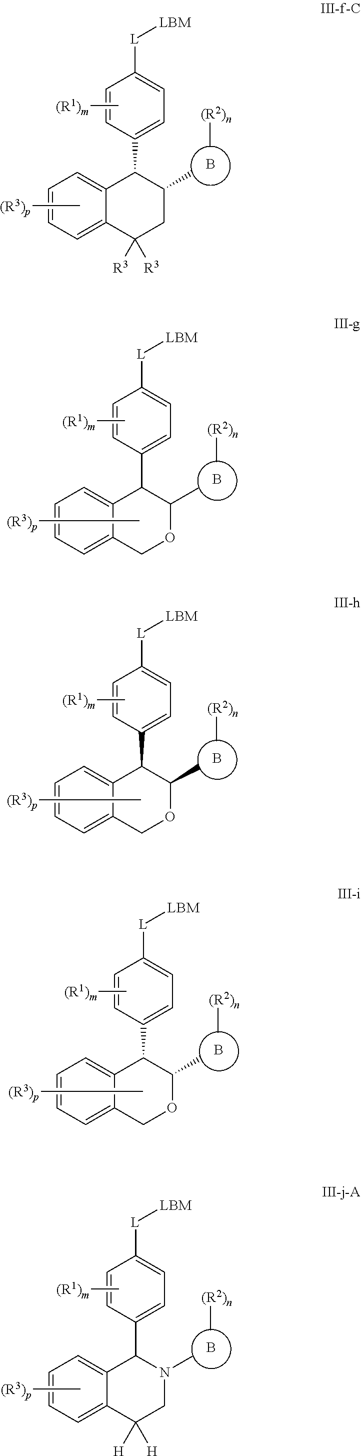

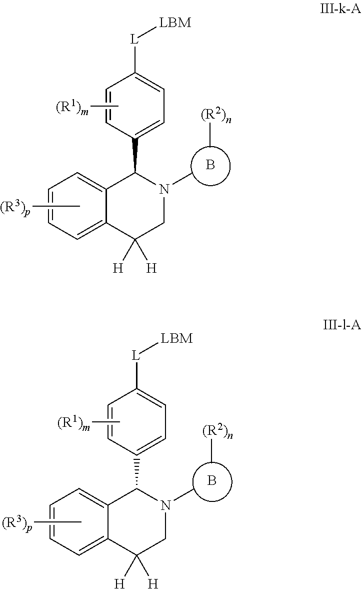

or a pharmaceutically acceptable salt thereof, wherein each of Ring B, R¹, R², R³, m, n, p, L, and LBM is as defined in embodiments and classes and subclasses herein.

In some embodiments, when the compound is of formula III-d-A, III-e-A, III-f-A, III-j-A, III-k-A, or III-l-A, then Ring B is other than phenyl.

In some embodiments, the compound is a compound of formula III-d-A, or a pharmaceutically acceptable salt thereof. In some embodiments, Ring B is other than phenyl.

In some embodiments, the compound is a compound of formula III-e-A, or a pharmaceutically acceptable salt thereof. In some embodiments, Ring B is other than phenyl.

In some embodiments, the compound is a compound of formula III-f-A, or a pharmaceutically acceptable salt thereof. In some embodiments, Ring B is other than phenyl.

In some embodiments, the compound is a compound of formula III-d-B, or a pharmaceutically acceptable salt thereof.

In some embodiments, the compound is a compound of formula III-e-B, or a pharmaceutically acceptable salt thereof.

In some embodiments, the compound is a compound of formula III-f-B, or a pharmaceutically acceptable salt thereof.

In some embodiments, the compound is a compound of formula III-d-C, or a pharmaceutically acceptable salt thereof.

In some embodiments, the compound is a compound of formula III-e-C, or a pharmaceutically acceptable salt thereof.

In some embodiments, the compound is a compound of formula III-f-C, or a pharmaceutically acceptable salt thereof.

In some embodiments, the compound is a compound of formula III-g, or a pharmaceutically acceptable salt thereof.

In some embodiments, the compound is a compound of formula III-h, or a pharmaceutically acceptable salt thereof.

In some embodiments, the compound is a compound of formula III-i, or a pharmaceutically acceptable salt thereof.

In some embodiments, the compound is a compound of formula III-j-A, or a pharmaceutically acceptable salt thereof. In some embodiments, Ring B is other than phenyl.

In some embodiments, the compound is a compound of formula III-k-A, or a pharmaceutically acceptable salt thereof. In some embodiments, Ring B is other than phenyl.

In some embodiments, the compound is a compound of formula III-l-A, or a pharmaceutically acceptable salt thereof. In some embodiments, Ring B is other than phenyl.

In certain embodiments, the present invention provides a compound of formula I-1, I-1′, I-2, I-2′, I-3 or I-3′, wherein ERBM IS

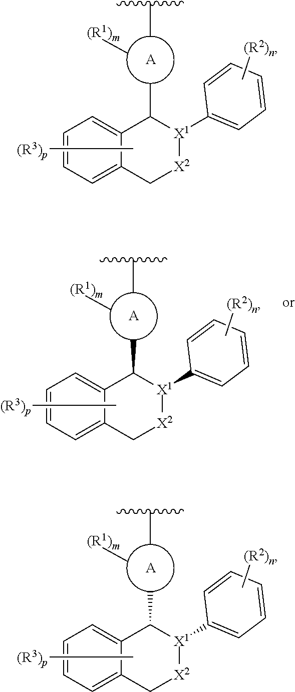

thereby forming a compound of one of formulas III-m, III-n, or III-o, respectively:

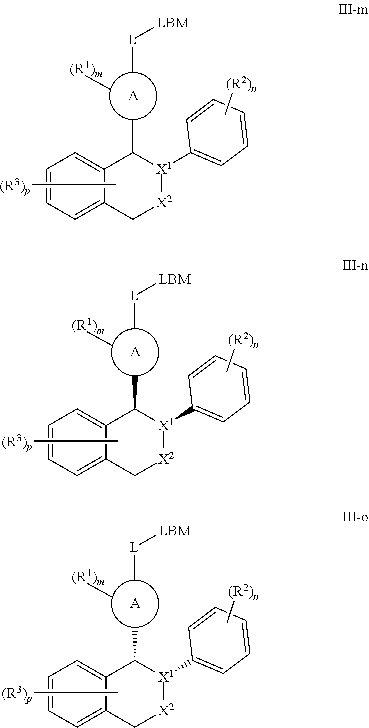

or a pharmaceutically acceptable salt thereof, wherein each of Ring A, R¹, R², R³, X¹, X², m, n, p, L, and LBM is as defined in embodiments and classes and subclasses herein.

In some embodiments, when X¹ is CH or N and X² is CH₂, then Ring A is other than phenyl or a 6 membered monocyclic heteroaryl ring having 1-3 nitrogen heteroatoms.

In some embodiments, the compound is a compound of formula III-m, or a pharmaceutically acceptable salt thereof. In some embodiments, when X¹ is CH or N and X² is CH₂, then Ring A is other than phenyl or a 6 membered monocyclic heteroaryl ring having 1-3 nitrogen heteroatoms.

In some embodiments, the compound is a compound of formula III-n, or a pharmaceutically acceptable salt thereof. In some embodiments, when X¹ is CH or N and X² is CH₂, then Ring A is other than phenyl or a 6 membered monocyclic heteroaryl ring having 1-3 nitrogen heteroatoms.

In some embodiments, the compound is a compound of formula III-o, or a pharmaceutically acceptable salt thereof. In some embodiments, when X¹ is CH or N and X² is CH₂, then Ring A is other than phenyl or a 6 membered monocyclic heteroaryl ring having 1-3 nitrogen heteroatoms.

In certain embodiments, the present invention provides a compound of formula I-1, I-1′, 1-2, I-2′, I-3 or I-3′, wherein ERBM is

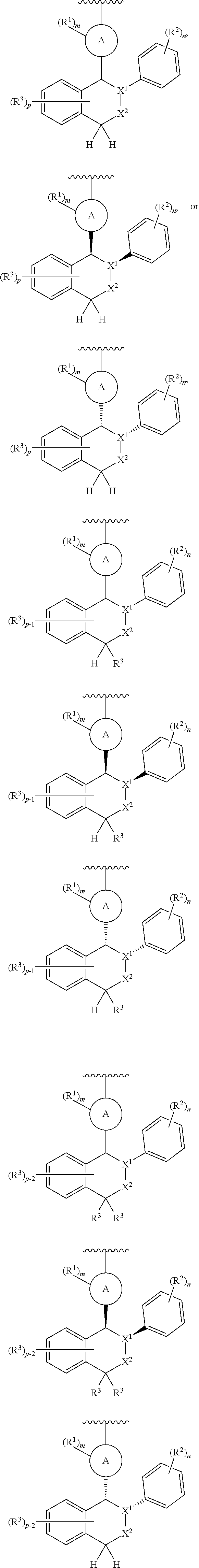

thereby forming a compound of one of formulas III-m-A, III-n-A, or III-o-A, III-m-B, III-n-B, III-o-B, III-m-C, III-n-C, or III-o-C:

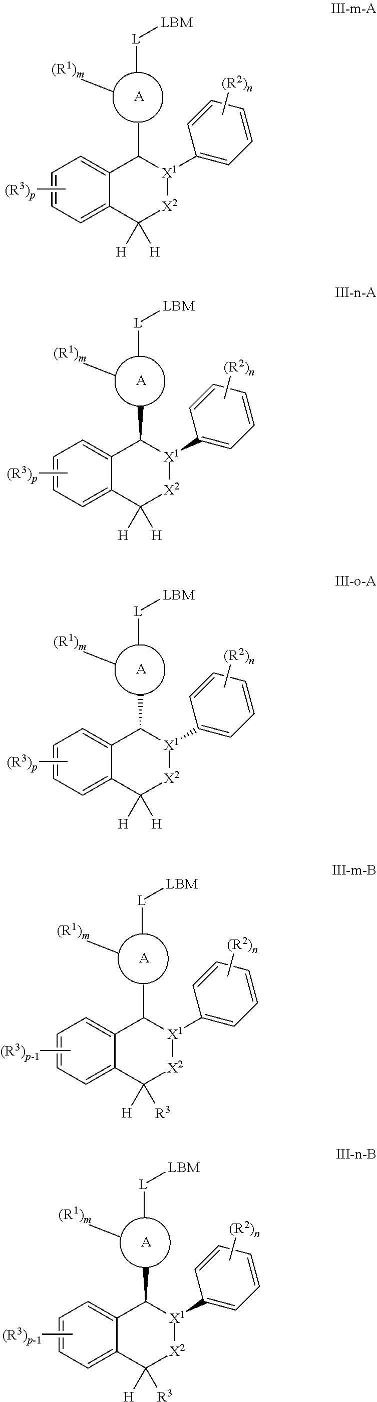

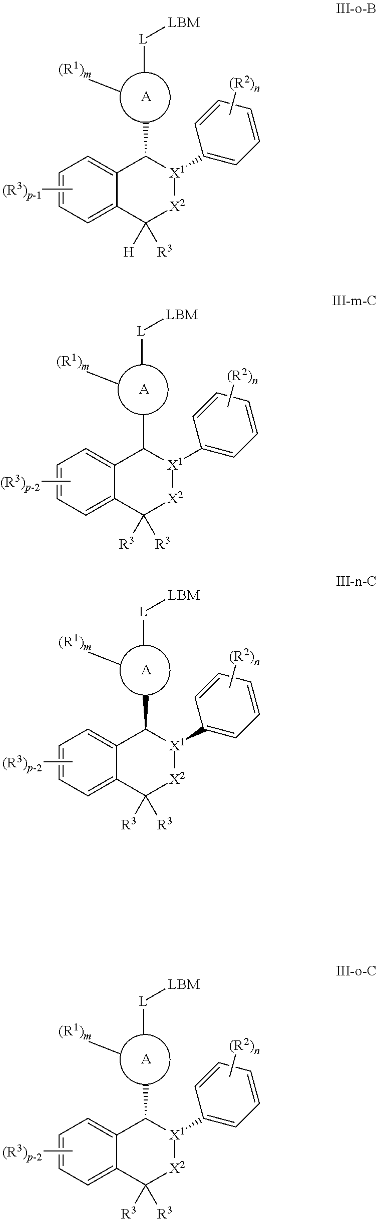

or a pharmaceutically acceptable salt thereof, wherein each of Ring B, R¹, R², R³, m, n, p, L, and LBM is as defined in embodiments and classes and subclasses herein.

In some embodiments, when the compound is of formulae III-m-A, III-n-A or III-o-A and X¹ is CH or N and X² is CH₂, then Ring A is other than phenyl or a 6 membered monocyclic heteroaryl ring having 1-3 nitrogen heteroatoms.

In some embodiments, the compound is a compound of formula III-m-A, or a pharmaceutically acceptable salt thereof. In some embodiments, when X¹ is CH or N and X² is CH₂, then Ring A is other than phenyl or a 6 membered monocyclic heteroaryl ring having 1-3 nitrogen heteroatoms.

In some embodiments, the compound is a compound of formula III-n-A, or a pharmaceutically acceptable salt thereof. In some embodiments, when X¹ is CH or N and X² is CH₂, then Ring A is other than phenyl or a 6 membered monocyclic heteroaryl ring having 1-3 nitrogen heteroatoms.

In some embodiments, the compound is a compound of formula III-o-A, or a pharmaceutically acceptable salt thereof. In some embodiments, when X¹ is CH or N and X² is CH₂, then Ring A is other than phenyl or a 6 membered monocyclic heteroaryl ring having 1-3 nitrogen heteroatoms.

In some embodiments, the compound is a compound of formula III-m-B, or a pharmaceutically acceptable salt thereof

In some embodiments, the compound is a compound of formula III-n-B, or a pharmaceutically acceptable salt thereof.

In some embodiments, the compound is a compound of formula III-o-B, or a pharmaceutically acceptable salt thereof.

In some embodiments, the compound is a compound of formula III-m-C, or a pharmaceutically acceptable salt thereof

In some embodiments, the compound is a compound of formula III-n-C, or a pharmaceutically acceptable salt thereof.

In some embodiments, the compound is a compound of formula III-o-C, or a pharmaceutically acceptable salt thereof.

In certain embodiments, the present invention provides a compound of formula I-1, I-1′, I-2, I-2′, I-3 or I-3′, wherein ERBM is

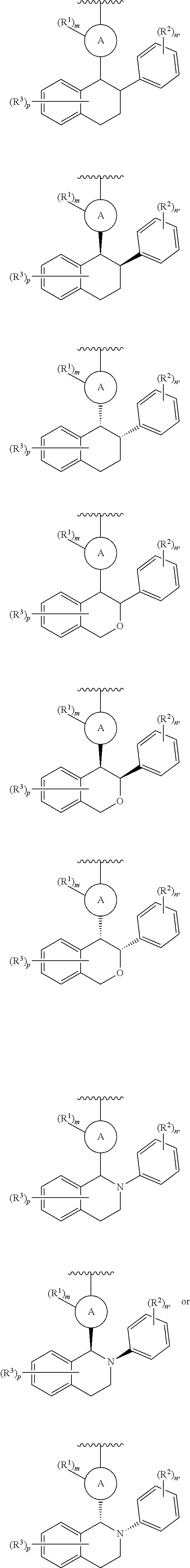

thereby forming a compound of one of formulas III-p, III-q, III-r, III-s, III-t, III-u, III-v, III-w, or III-x, respectively:

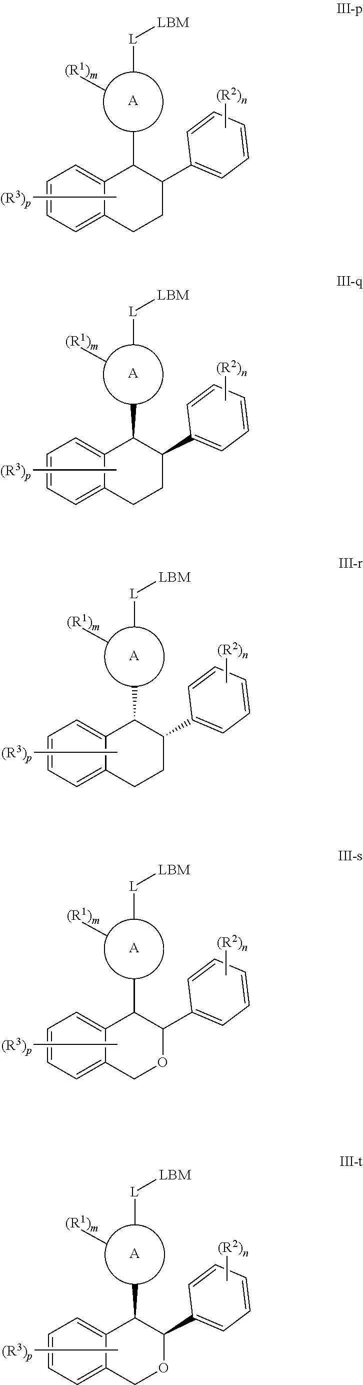

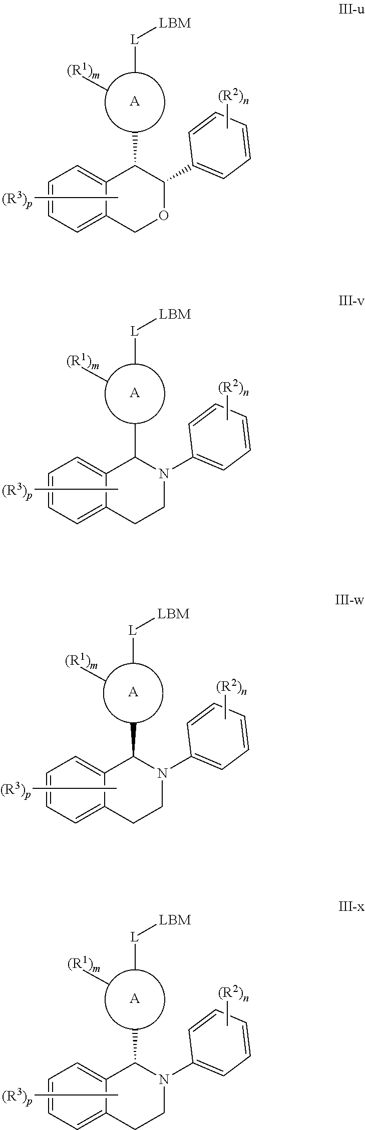

or a pharmaceutically acceptable salt thereof, wherein each of Ring A, R¹, R², R³, m, n, p, L, and LBM is as defined in embodiments and classes and subclasses herein. In some embodiments, when the compound is of formulas III-p. III-q, III-r, III-v, III-w or III-x, then Ring A is other than phenyl or a 6 membered monocyclic heteroaryl ring having 1-3 nitrogen heteroatoms.

In some embodiments, the compound is a compound of formula III-p, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-q, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-r, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-s, or a pharmaceutically acceptable salt thereof.

In some embodiments, the compound is a compound of formula III-t, or a pharmaceutically acceptable salt thereof.

In some embodiments, the compound is a compound of formula III-u, or a pharmaceutically acceptable salt thereof.

In some embodiments, the compound is a compound of formula III-v, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-w, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-x, or a pharmaceutically acceptable salt thereof. In some embodiments, Ring A is other than phenyl or a 6 membered monocyclic heteroaryl ring having 1-3 nitrogen heteroatoms.

In certain embodiments, the present invention provides a compound of formula I-1, I-1′, I-2, I-2′, I-3 or I-3′, wherein ERBM is

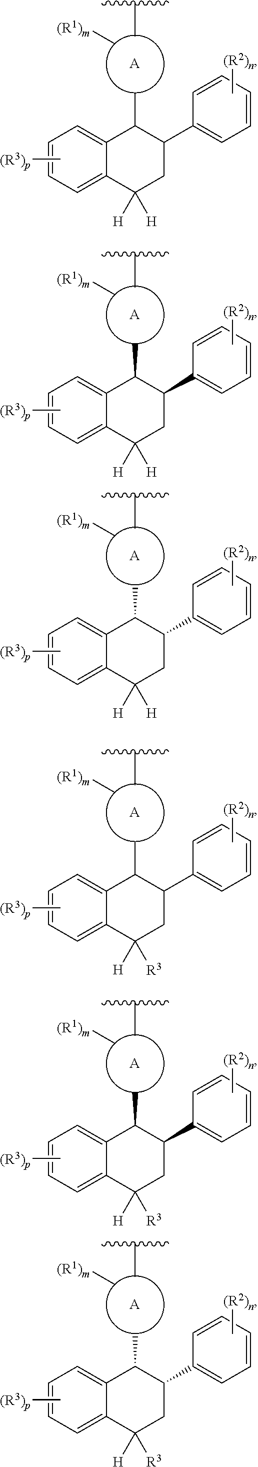

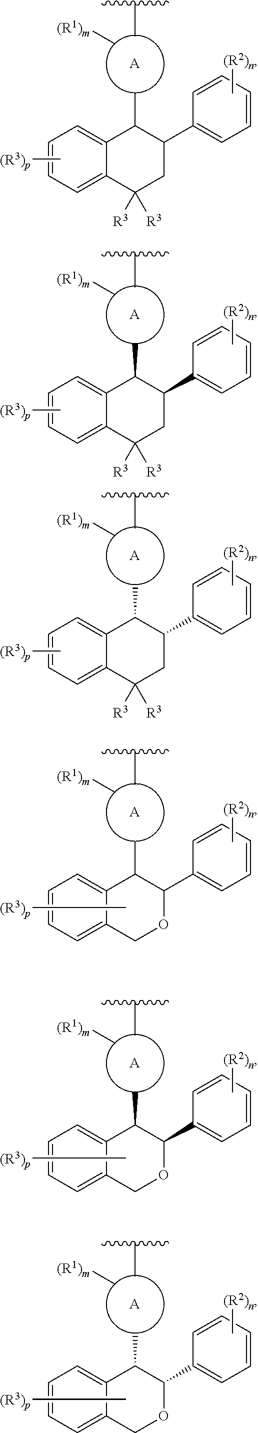

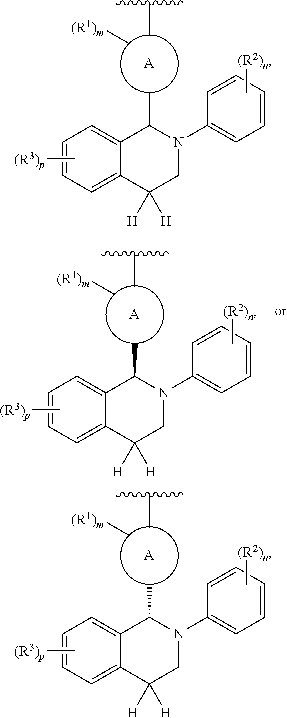

thereby forming a compound of one of formulas III-p-A, III-q-A, III-r-A, III-p-B, III-q-B, III-r-B, III-p-C, III-q-C, III-r-C, III-s, III-t, III-u, III-v-A, III-w-A, or III-x-A, respectively:

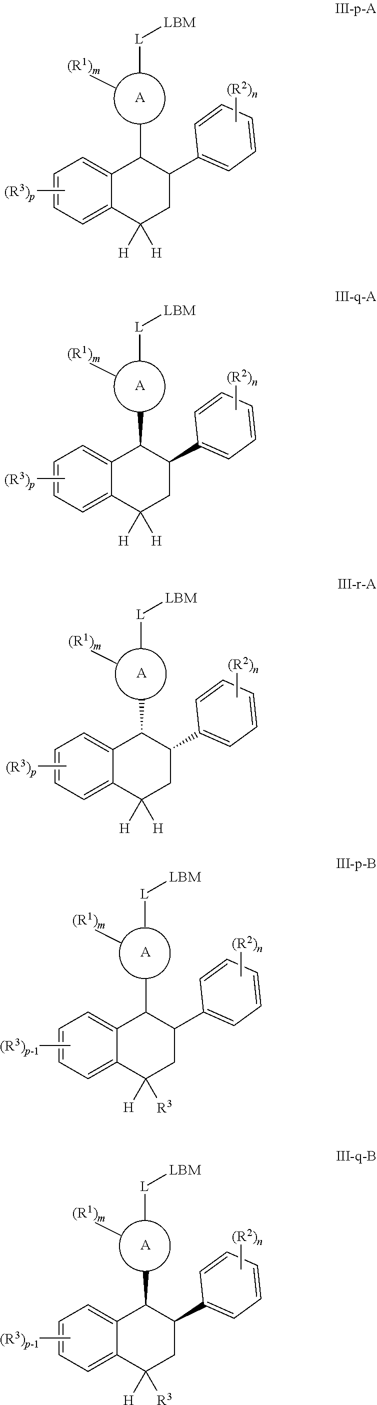

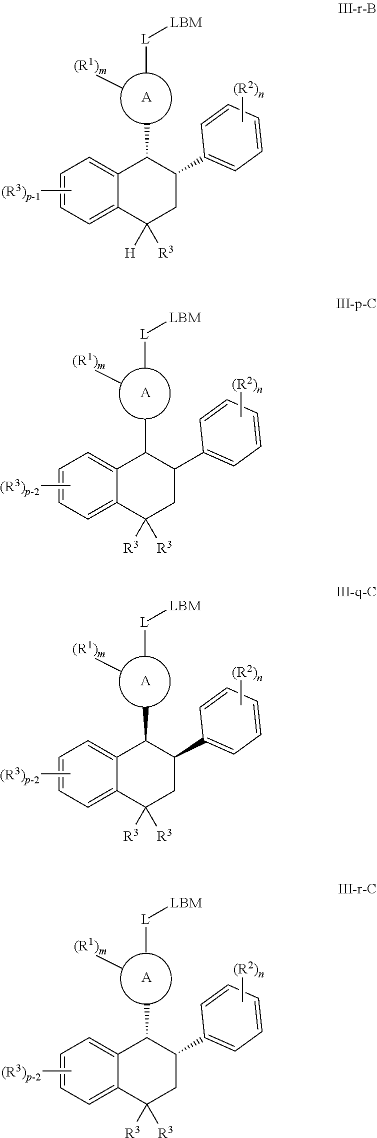

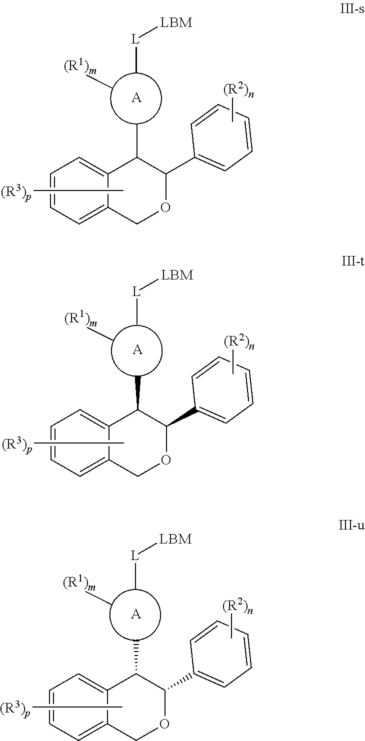

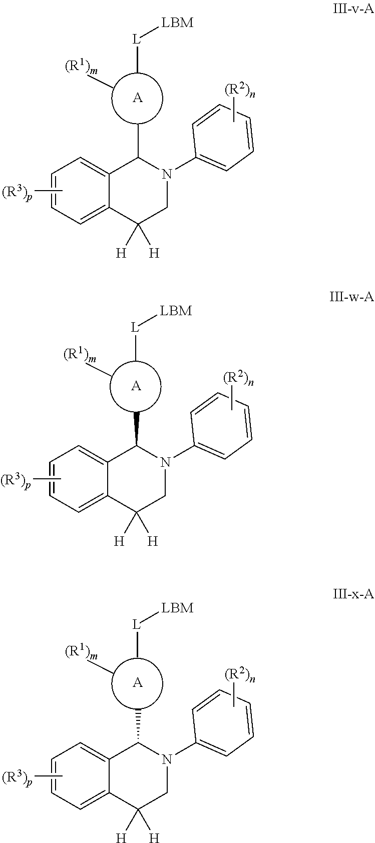

or a pharmaceutically acceptable salt thereof, wherein each of Ring A, R¹, R², R³, m, n, p, L, and LBM is as defined in embodiments and classes and subclasses herein.

In some embodiments, when the compound is of formulas III-p-A, III-q-A, III-r-A, III-v-A, III-w-A or III-x-A, then Ring A is other than phenyl or a 6 membered monocyclic heteroaryl ring having 1-3 nitrogen heteroatoms.

In some embodiments, the compound is a compound of formula III-p-A, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-q-A, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-r-A, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-p-B, or a pharmaceutically acceptable salt thereof.

In some embodiments, the compound is a compound of formula III-q-B, or a pharmaceutically acceptable salt thereof.

In some embodiments, the compound is a compound of formula III-r-B, or a pharmaceutically acceptable salt thereof.

In some embodiments, the compound is a compound of formula III-p-C, or a pharmaceutically acceptable salt thereof.

In some embodiments, the compound is a compound of formula III-q-C, or a pharmaceutically acceptable salt thereof.

In some embodiments, the compound is a compound of formula III-r-C, or a pharmaceutically acceptable salt thereof.

In some embodiments, the compound is a compound of formula III-v-A, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-w-A, or a pharmaceutically acceptable salt thereof. In some embodiments, Ring A is other than phenyl or a 6 membered monocyclic heteroaryl ring having 1-3 nitrogen heteroatoms.

In some embodiments, the compound is a compound of formula III-x-A, or a pharmaceutically acceptable salt thereof. In some embodiments, Ring A is other than phenyl or a 6 membered monocyclic heteroaryl ring having 1-3 nitrogen heteroatoms.

In certain embodiments, the present invention provides a compound of formula I-1, I-1′, I-2, I-2′, I-3 or I-3′, wherein ERBM IS

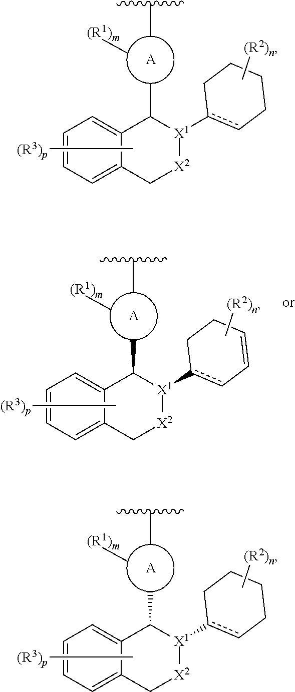

thereby forming a compound of one of formulas IV-a, IV-b, or IV-c, respectively:

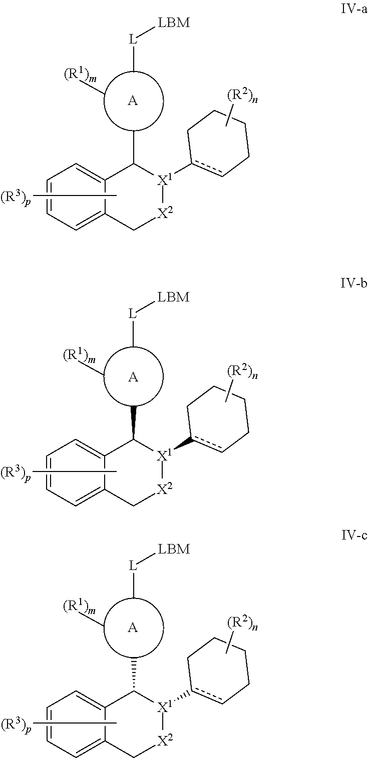

or a pharmaceutically acceptable salt thereof, wherein [symbol] represents a single or double bond and each of Ring A, R¹, R², R³, X¹, X², m, n, p, L, and LBM is as defined in embodiments and classes and subclasses herein.

In some embodiments, the compound is a compound of formula IV-a, or a pharmaceutically acceptable salt thereof.

In some embodiments, the compound is a compound of formula IV-b, or a pharmaceutically acceptable salt thereof.

In some embodiments, the compound is a compound of formula IV-b, or a pharmaceutically acceptable salt thereof.

In certain embodiments, the present invention provides a compound of formula I-1, I-1′, I-2, I-2′, I-3 or I-3′, wherein ERBM is

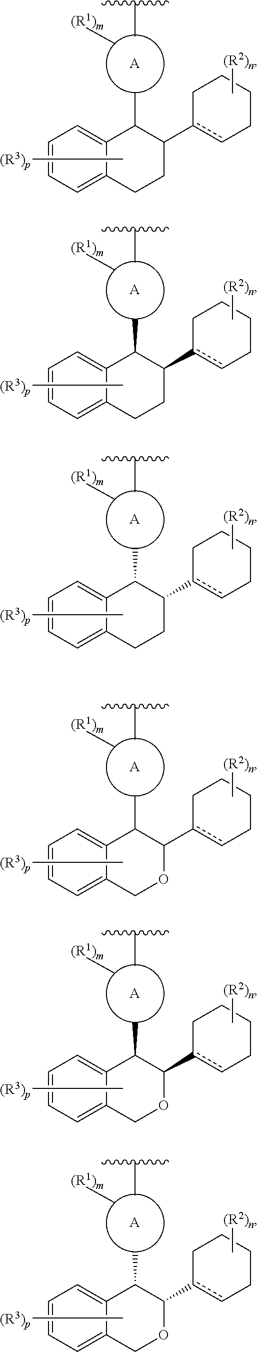

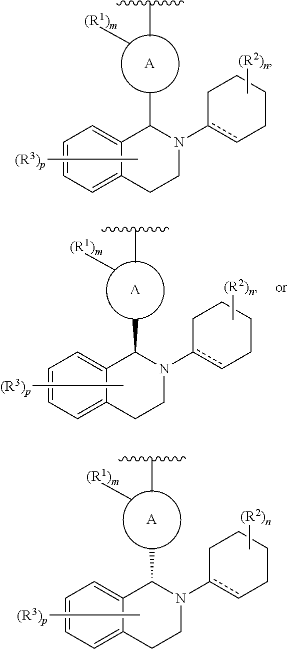

thereby forming a compound of one of formulas IV-d, IV-e, IV-f, IV-g, IV-h, IV-i, IV-j, IV-k, or IV-l, respectively:

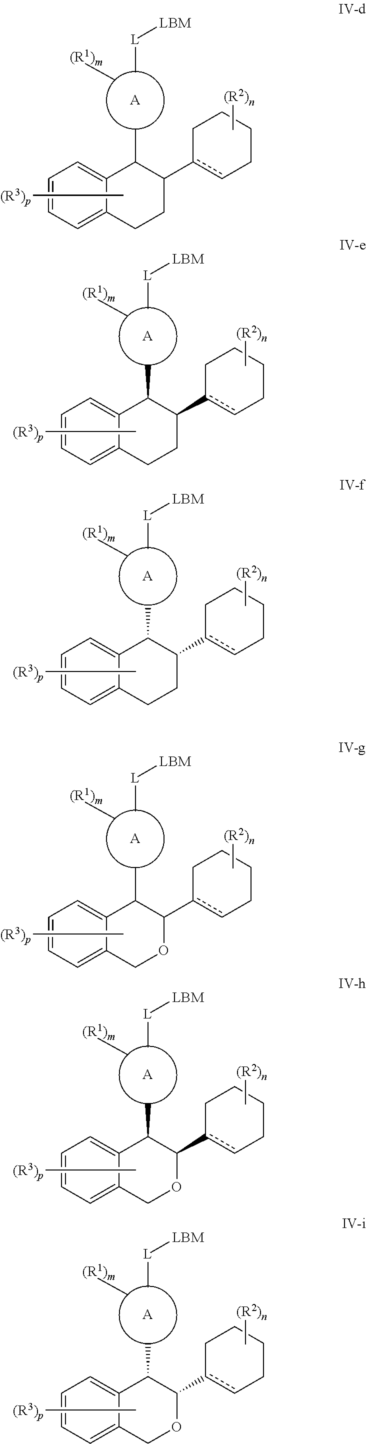

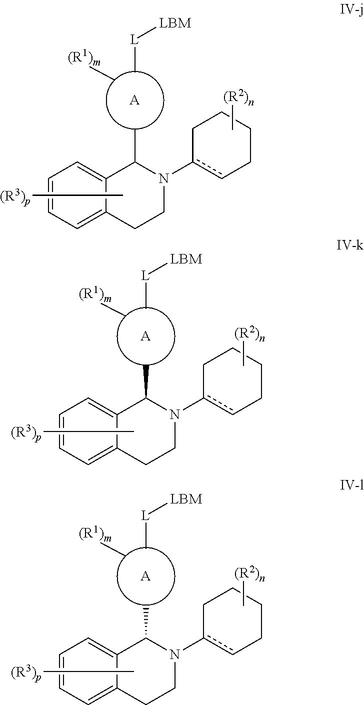

or a pharmaceutically acceptable salt thereof, wherein [symbol] represents a single or double bond and each of Ring A, R¹, R², R³, m, n, p, L, and LBM is as defined in embodiments and classes and subclasses herein.

In some embodiments, the compound is a compound of formula IV-d, or a pharmaceutically acceptable salt thereof.

In some embodiments, the compound is a compound of formula IV-e, or a pharmaceutically acceptable salt thereof.

In some embodiments, the compound is a compound of formula IV-f, or a pharmaceutically acceptable salt thereof.

In some embodiments, the compound is a compound of formula IV-g, or a pharmaceutically acceptable salt thereof.

In some embodiments, the compound is a compound of formula IV-h, or a pharmaceutically acceptable salt thereof.

In some embodiments, the compound is a compound of formula IV-i, or a pharmaceutically acceptable salt thereof.

In some embodiments, the compound is a compound of formula IV-j, or a pharmaceutically acceptable salt thereof.

In some embodiments, the compound is a compound of formula IV-k, or a pharmaceutically acceptable salt thereof.

In some embodiments, the compound is a compound of formula IV-l, or a pharmaceutically acceptable salt thereof.

In certain embodiments, the present invention provides a compound of formula I-1, I-1′, I-2, I-2′, I-3 or I-3′, wherein ERBM IS

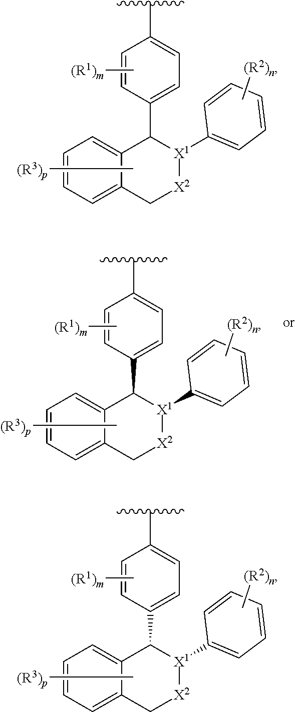

thereby forming a compound of one of formulas IV-m, IV-n, or IV-o, respectively:

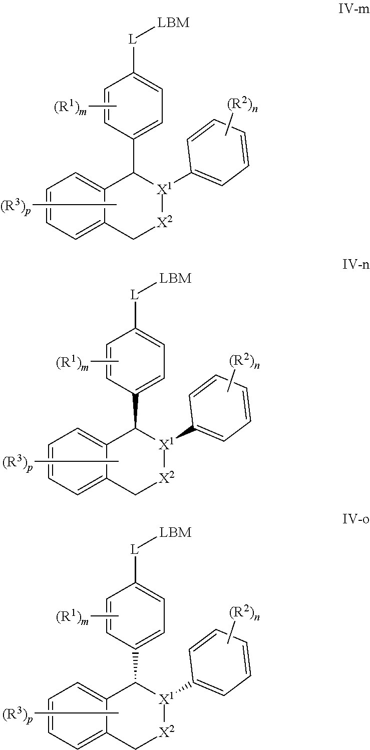

or a pharmaceutically acceptable salt thereof, wherein each of R¹, R², R³, X¹, X², m, n, p, L, and LBM is as defined in embodiments and classes and subclasses herein. In some embodiments, when X¹ is CH or N then X² is other than CH₂.

In some embodiments, the compound is a compound of formula IV-m, or a pharmaceutically acceptable salt thereof. In some embodiments, when X¹ is CH or N then X² is other than CH₂.

In some embodiments, the compound is a compound of formula IV-n, or a pharmaceutically acceptable salt thereof. In some embodiments, when X¹ is CH or N then X² is other than CH₂.

In some embodiments, the compound is a compound of formula IV-o, or a pharmaceutically acceptable salt thereof. In some embodiments, when X¹ is CH or N then X² is other than CH₂.

In certain embodiments, the present invention provides a compound of formula I-1, I-1′, I-2, I-2′, I-3 or I-3′, wherein ERBM is

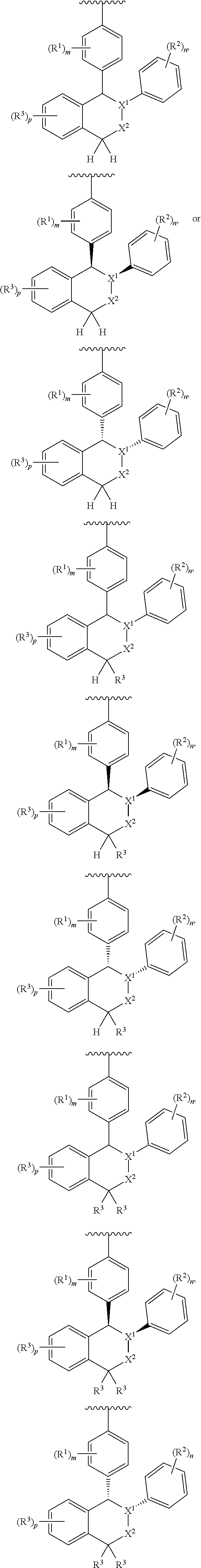

thereby forming a compound of one of formulas IV-m-A, IV-n-A, IV-o-A, IV-m-B, IV-n-B, IV-o-B, IV-m-C, IV-n-C, or IV-o-C, respectively:

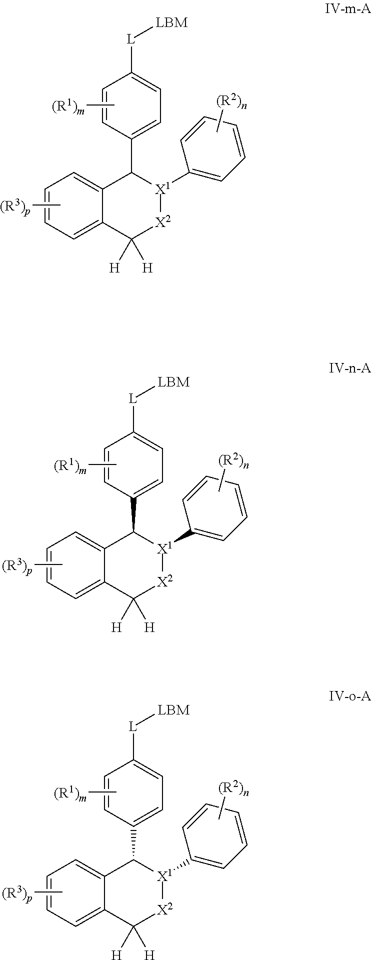

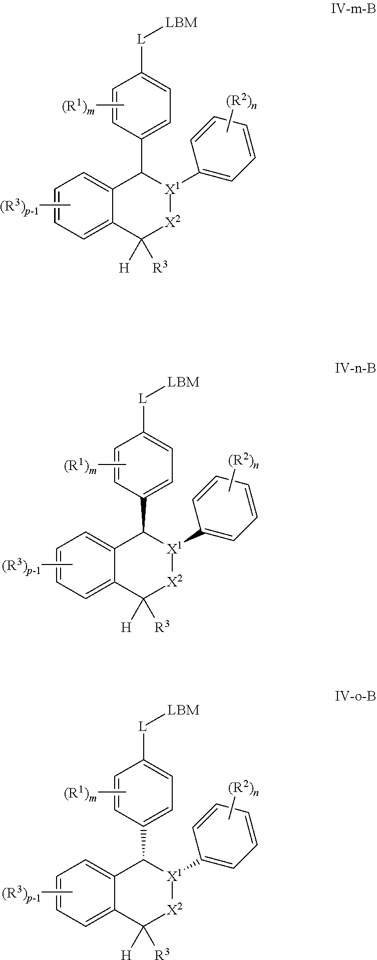

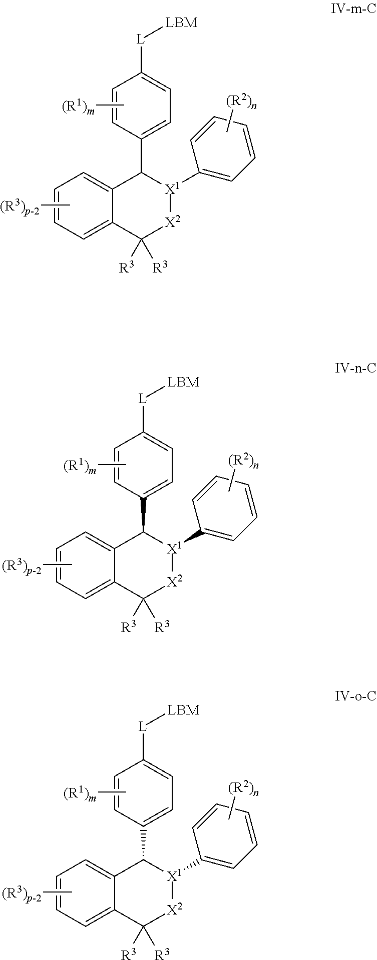

or a pharmaceutically acceptable salt thereof, wherein each of R¹, R², R³, X¹, X², m, n, p, L, and LBM is as defined in embodiments and classes and subclasses herein.

In some embodiments, when the compound is of formula IV-m-A, IV-n-A or IV-o-A and X¹ is CH or N then X² is other than CH₂.

In some embodiments, the compound is a compound of formula IV-m-A, or a pharmaceutically acceptable salt thereof. In some embodiments, when X¹ is CH or N then X² is other than CH₂.

In some embodiments, the compound is a compound of formula IV-n-A, or a pharmaceutically acceptable salt thereof. In some embodiments, when X¹ is CH or N then X² is other than CH₂.

In some embodiments, the compound is a compound of formula IV-o-A, or a pharmaceutically acceptable salt thereof. In some embodiments, when X¹ is CH or N then X² is other than CH₂.

In some embodiments, the compound is a compound of formula IV-m-B, or a pharmaceutically acceptable salt thereof.

In some embodiments, the compound is a compound of formula IV-n-B, or a pharmaceutically acceptable salt thereof.

In some embodiments, the compound is a compound of formula IV-o-B, or a pharmaceutically acceptable salt thereof.

In some embodiments, the compound is a compound of formula IV-m-C, or a pharmaceutically acceptable salt thereof.

In some embodiments, the compound is a compound of formula IV-n-C, or a pharmaceutically acceptable salt thereof.

In some embodiments, the compound is a compound of formula IV-o-C, or a pharmaceutically acceptable salt thereof.

In certain embodiments, the present invention provides a compound of formula I-1, I-1′, I-2, I-2′, I-3 or I-3′, wherein ERBM is

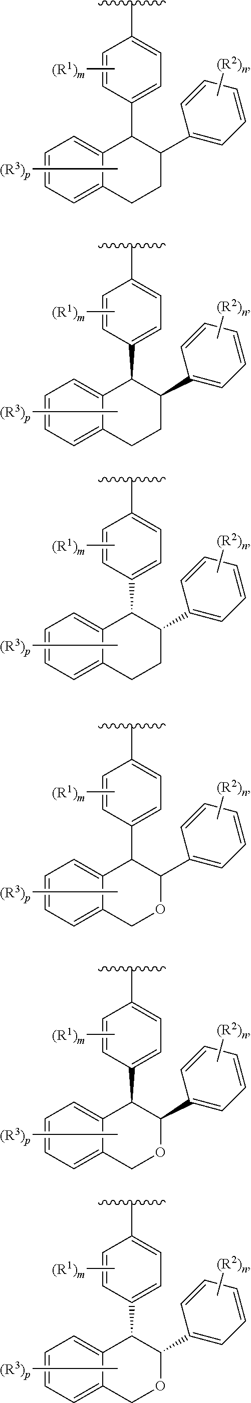

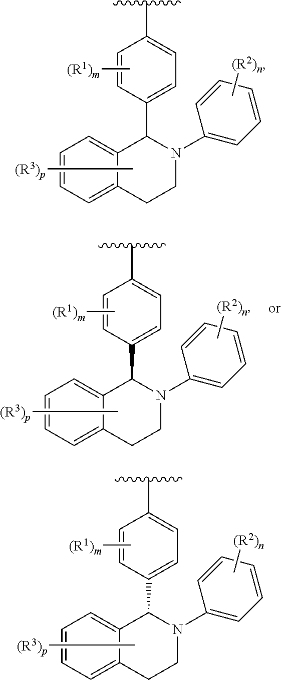

thereby forming a compound of one of formulas IV-p, IV-q. IV-r, IV-s, IV-t, IV-u, IV-v, IV-w, or IV-x, respectively:

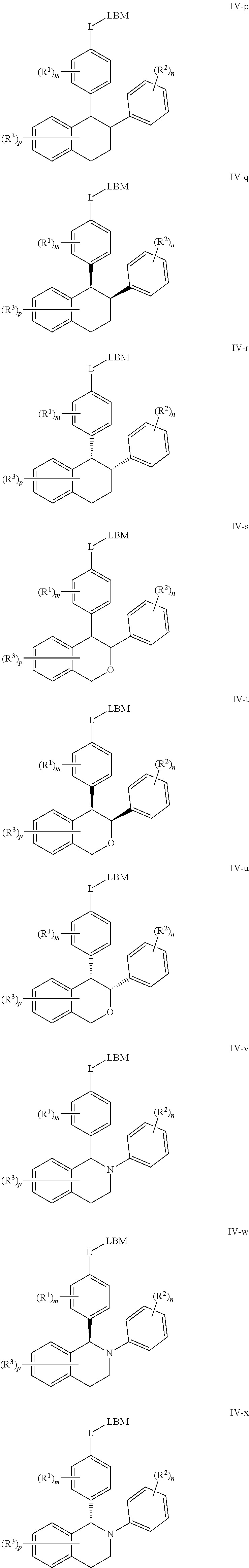

or a pharmaceutically acceptable salt thereof, wherein each of R¹, R², R³, m, n, p, L, and LBM is as defined in embodiments and classes and subclasses herein.

In some embodiments, the compound is a compound of formula IV-p, or a pharmaceutically acceptable salt thereof.

In some embodiments, the compound is a compound of formula IV-q, or a pharmaceutically acceptable salt thereof.

In some embodiments, the compound is a compound of formula IV-r, or a pharmaceutically acceptable salt thereof.

In some embodiments, the compound is a compound of formula IV-s, or a pharmaceutically acceptable salt thereof.

In some embodiments, the compound is a compound of formula IV-t, or a pharmaceutically acceptable salt thereof.

In some embodiments, the compound is a compound of formula IV-u, or a pharmaceutically acceptable salt thereof.

In some embodiments, the compound is a compound of formula IV-v, or a pharmaceutically acceptable salt thereof.

In some embodiments, the compound is a compound of formula IV-w, or a pharmaceutically acceptable salt thereof.

In some embodiments, the compound is a compound of formula IV-x, or a pharmaceutically acceptable salt thereof.

In certain embodiments, the present invention provides a compound of formula I-1, I-1′, I-2, I-2′, I-3 or I-3′, wherein ERBM is

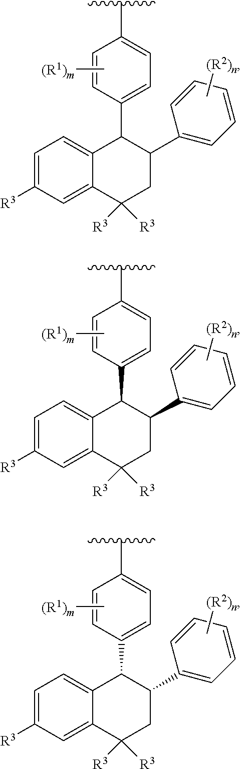

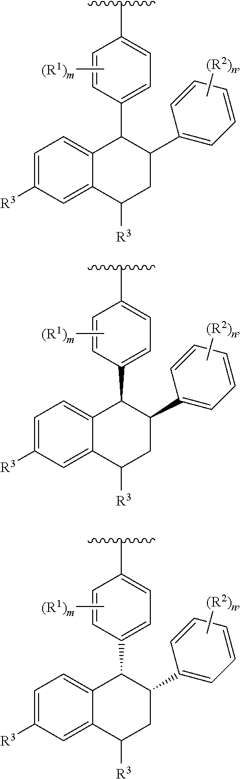

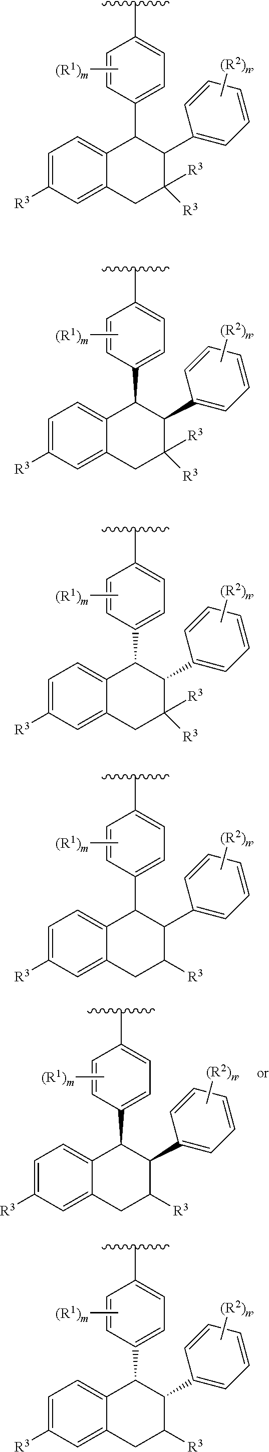

thereby forming a compound of one of formulas IV-aa, IV-bb, IV-cc, IV-dd, IV-ee, IV-ff, IV-gg, IV-hh, IV-ii, IV-jj, IV-kk, or IV-ll, respectively:

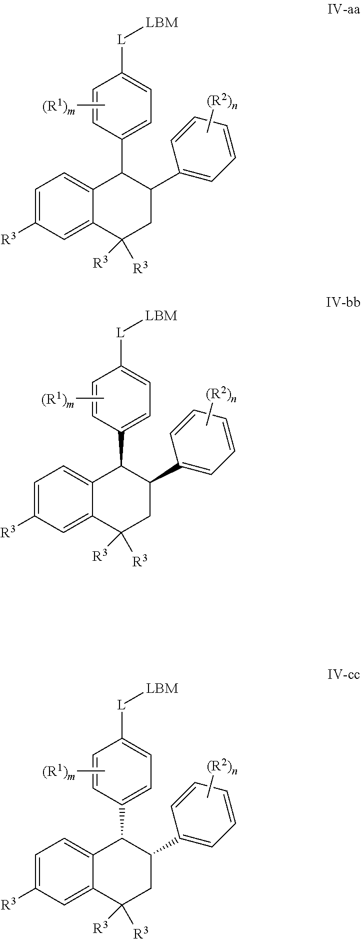

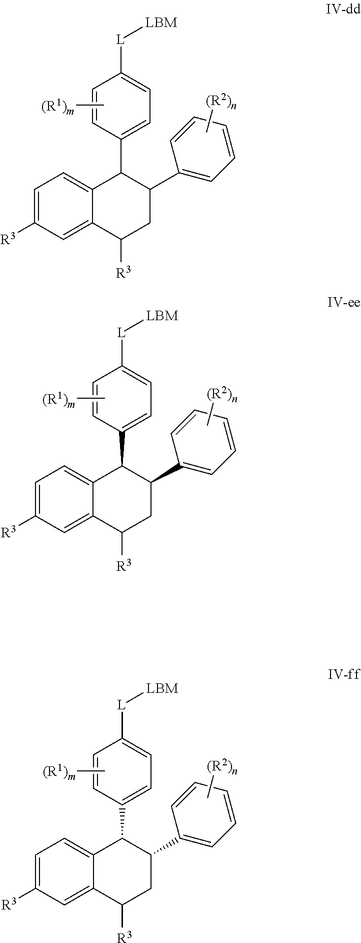

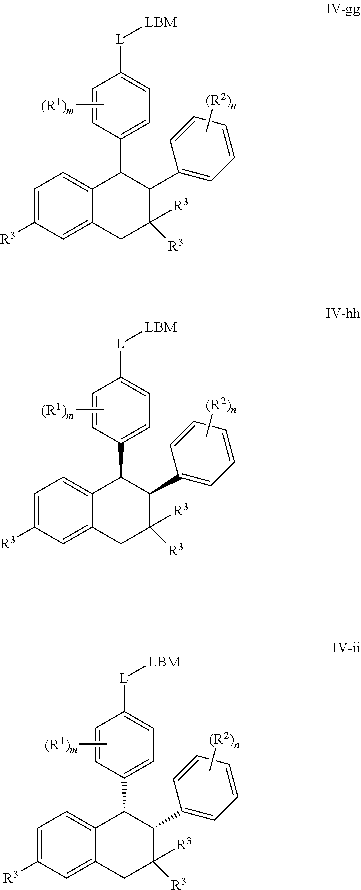

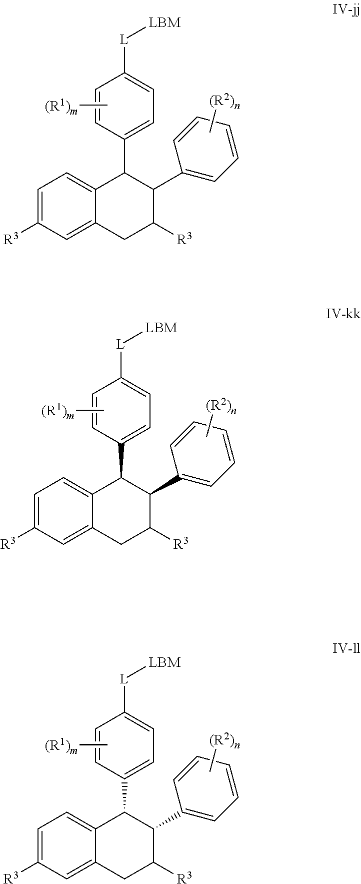

or a pharmaceutically acceptable salt thereof, wherein each of R¹, R², R³, m, n, p, L, and LBM is as defined in embodiments and classes and subclasses herein.

In some embodiments, the compound is a compound of formula IV-aa, or a pharmaceutically acceptable salt thereof.

In some embodiments, the compound is a compound of formula IV-bb, or a pharmaceutically acceptable salt thereof.

In some embodiments, the compound is a compound of formula IV-cc, or a pharmaceutically acceptable salt thereof.

In some embodiments, the compound is a compound of formula IV-dd, or a pharmaceutically acceptable salt thereof.

In some embodiments, the compound is a compound of formula IV-ee, or a pharmaceutically acceptable salt thereof.

In some embodiments, the compound is a compound of formula IV-ff, or a pharmaceutically acceptable salt thereof.

In some embodiments, the compound is a compound of formula IV-gg, or a pharmaceutically acceptable salt thereof.

In some embodiments, the compound is a compound of formula IV-hh, or a pharmaceutically acceptable salt thereof.

In some embodiments, the compound is a compound of formula IV-ii, or a pharmaceutically acceptable salt thereof.

In some embodiments, the compound is a compound of formula IV-jj, or a pharmaceutically acceptable salt thereof.

In some embodiments, the compound is a compound of formula IV-kk, or a pharmaceutically acceptable salt thereof.

In some embodiments, the compound is a compound of formula IV-ll, or a pharmaceutically acceptable salt thereof.

In certain embodiments, the present invention provides a compound of formula I-1, I-1′, I-2, I-2′, I-3 or I-3′, wherein ERBM is

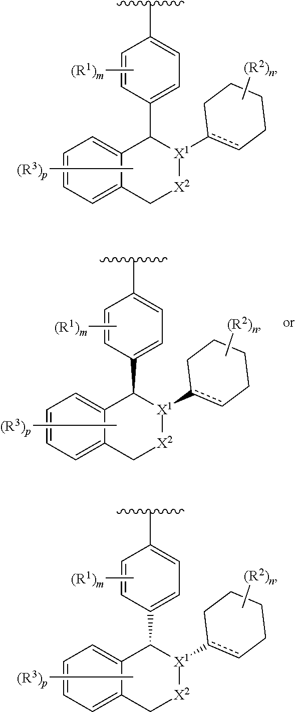

thereby forming a compound of one of formulas V-a, V-b, or V-c, respectively:

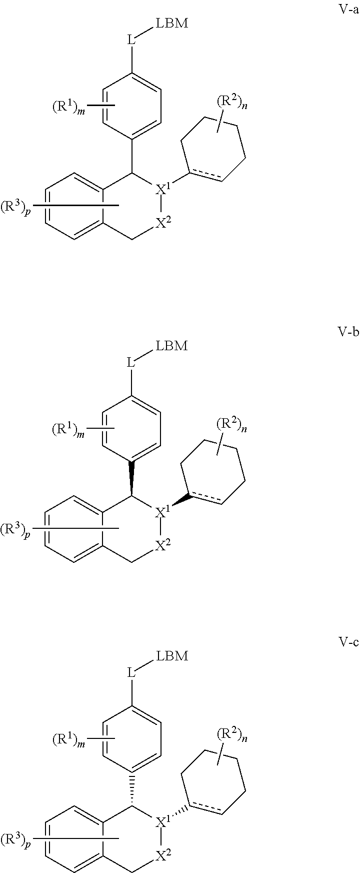

or a pharmaceutically acceptable salt thereof, wherein [symbol] represents a single or double bond and each of R¹, R², R³, X¹, X², m, n, p, L, and LBM is as defined in embodiments and classes and subclasses herein.

In some embodiments, the compound is a compound of formula V-a, or a pharmaceutically acceptable salt thereof.

In some embodiments, the compound is a compound of formula V-b, or a pharmaceutically acceptable salt thereof.

In some embodiments, the compound is a compound of formula V-c, or a pharmaceutically acceptable salt thereof.

In certain embodiments, the present invention provides a compound of formula I-1, I-1′, I-2, I-2′, I-3 or I-3′, wherein ERBM is

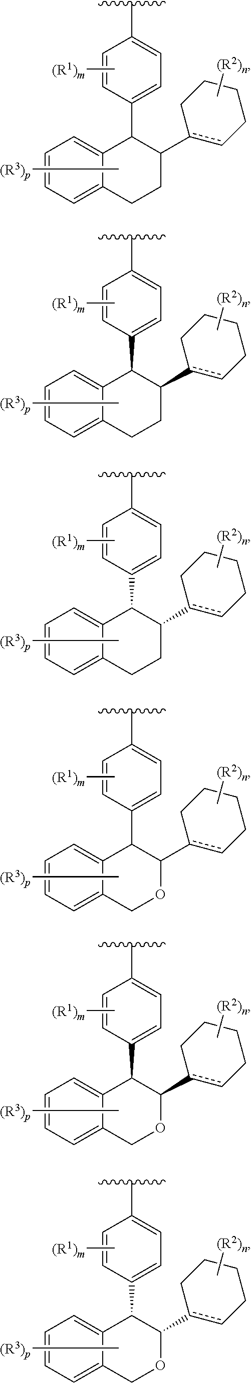

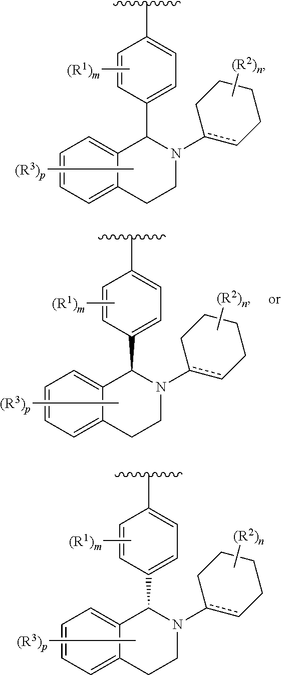

thereby forming a compound of one of formulas V-d, V-e, V-f, V-g. V-h, V-i, V-j, V-k, or V-l, respectively:

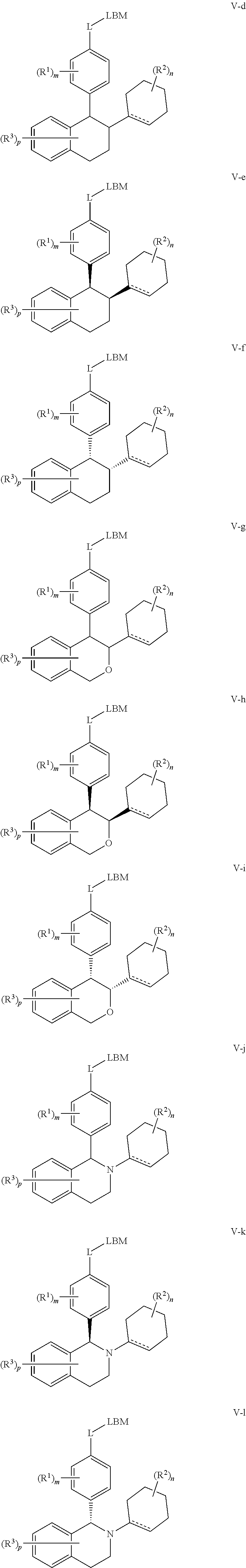

or a pharmaceutically acceptable salt thereof, wherein [symbol] represents a single or double bond and each of R¹, R², R³, m, n, p, L, and LBM is as defined in embodiments and classes and subclasses herein.

In some embodiments, the compound is a compound of formula V-d, or a pharmaceutically acceptable salt thereof.

In some embodiments, the compound is a compound of formula V-e, or a pharmaceutically acceptable salt thereof.

In some embodiments, the compound is a compound of formula V-f, or a pharmaceutically acceptable salt thereof.

In some embodiments, the compound is a compound of formula V-g, or a pharmaceutically acceptable salt thereof.

In some embodiments, the compound is a compound of formula V-h, or a pharmaceutically acceptable salt thereof.

In some embodiments, the compound is a compound of formula V-i, or a pharmaceutically acceptable salt thereof.

In some embodiments, the compound is a compound of formula V-j, or a pharmaceutically acceptable salt thereof.

In some embodiments, the compound is a compound of formula V-k, or a pharmaceutically acceptable salt thereof.

In some embodiments, the compound is a compound of formula V-l, or a pharmaceutically acceptable salt thereof.

In certain embodiments, the present invention provides a compound of formula I-1, I-1′, I-2, I-2′, I-3 or I-3′, wherein ERBM is

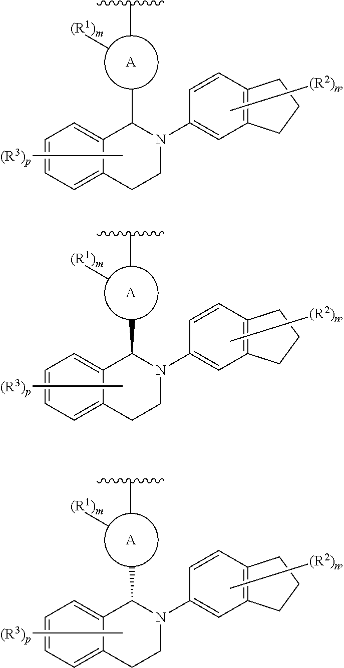

thereby forming a compound of one of formulas VI-a, VI-b, or VI-c, respectively:

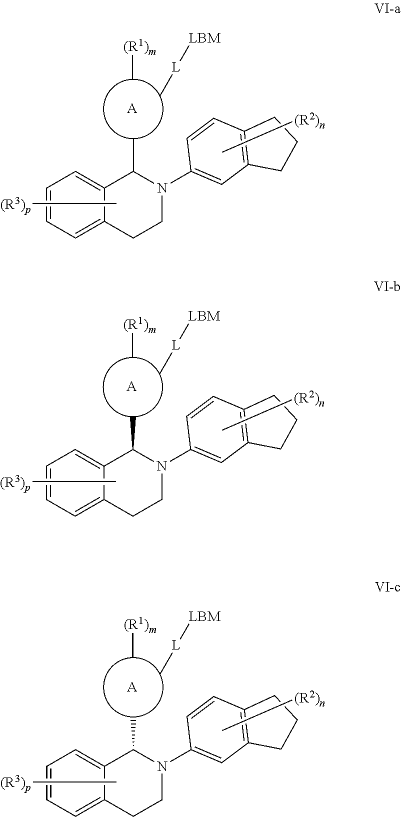

or a pharmaceutically acceptable salt thereof, wherein each of Ring A, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM is

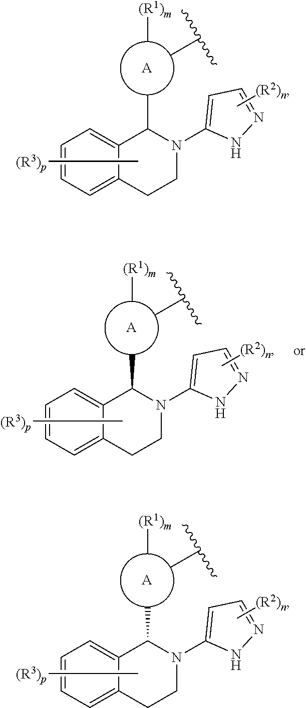

thereby forming a compound of one of formulas VI-d, VI-e, or VI-f, respectively:

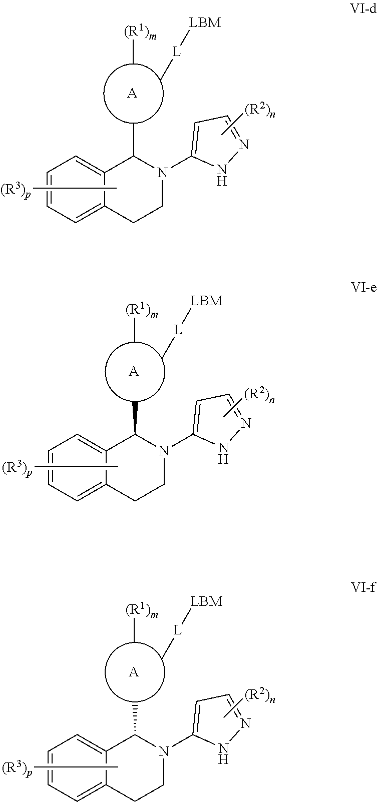

or a pharmaceutically acceptable salt thereof, wherein each of Ring A, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM is

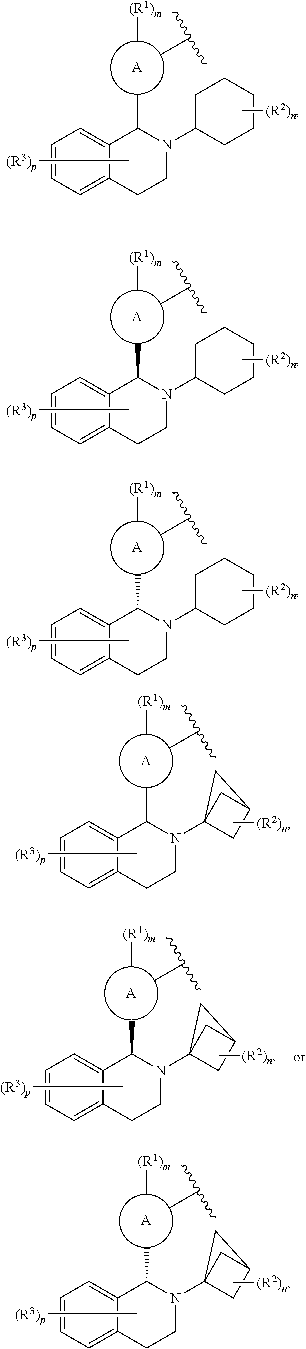

thereby forming a compound of one of formulas VI-g, VI-h, VI-i, VI-j, VI-k, or VI-l, respectively:

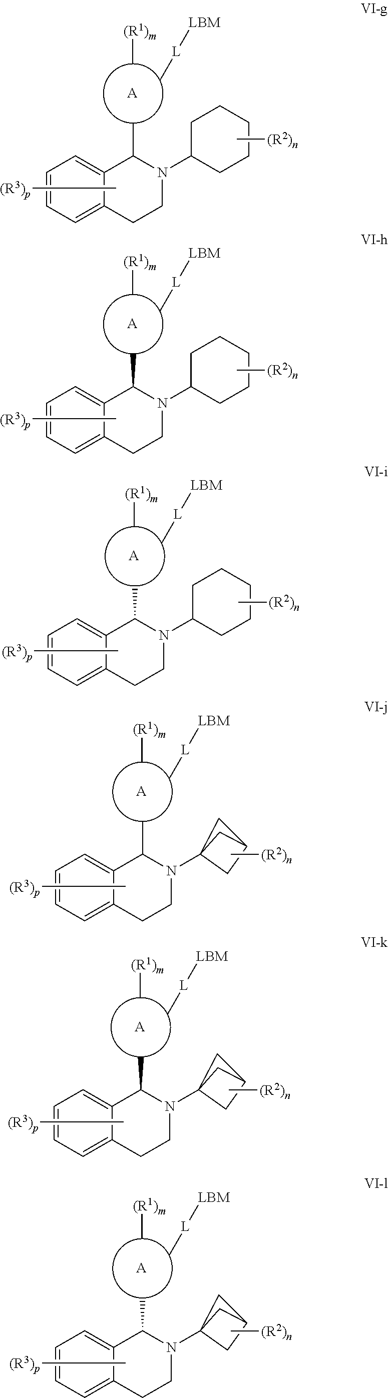

or a pharmaceutically acceptable salt thereof, wherein each of Ring A, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM is

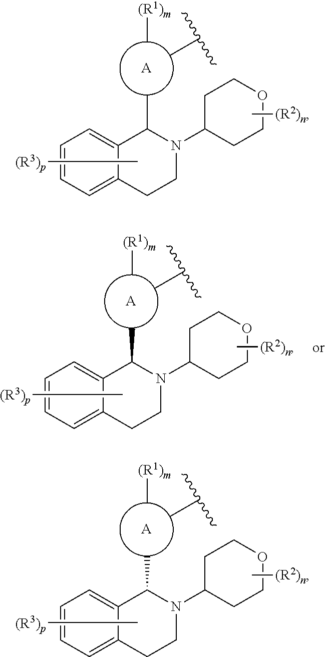

thereby forming a compound of one of formulas VI-m, VI-n, or VI-o, respectively:

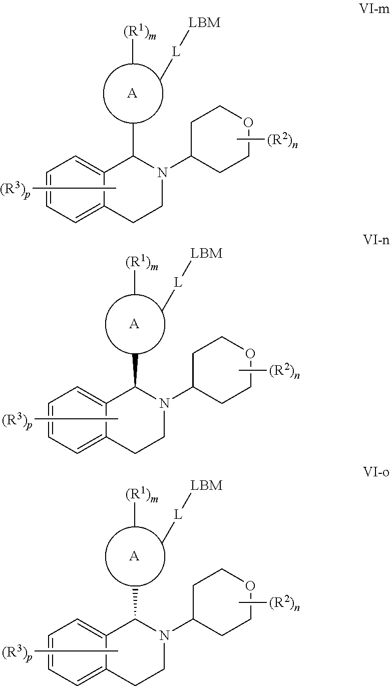

or a pharmaceutically acceptable salt thereof, wherein each of Ring A, R¹, R², R³, m, n, p, L, and LBM is as defined in embodiments and classes and subclasses herein.

In some embodiments, the compound is a compound of formula VI-a, or a pharmaceutically acceptable salt thereof.

In some embodiments, the compound is a compound of formula VI-b, or a pharmaceutically acceptable salt thereof.

In some embodiments, the compound is a compound of formula VI-c, or a pharmaceutically acceptable salt thereof.

In some embodiments, the compound is a compound of formula VI-d, or a pharmaceutically acceptable salt thereof.

In some embodiments, the compound is a compound of formula VI-e, or a pharmaceutically acceptable salt thereof.

In some embodiments, the compound is a compound of formula VI-f, or a pharmaceutically acceptable salt thereof.

In some embodiments, the compound is a compound of formula VI-g, or a pharmaceutically acceptable salt thereof.

In some embodiments, the compound is a compound of formula VI-h, or a pharmaceutically acceptable salt thereof.

In some embodiments, the compound is a compound of formula VI-i, or a pharmaceutically acceptable salt thereof.

In some embodiments, the compound is a compound of formula VI-j, or a pharmaceutically acceptable salt thereof.

In some embodiments, the compound is a compound of formula VI-k, or a pharmaceutically acceptable salt thereof.

In some embodiments, the compound is a compound of formula VI-l, or a pharmaceutically acceptable salt thereof.

In some embodiments, the compound is a compound of formula VI-m, or a pharmaceutically acceptable salt thereof.

In some embodiments, the compound is a compound of formula VI-n, or a pharmaceutically acceptable salt thereof.

In some embodiments, the compound is a compound of formula VI-o, or a pharmaceutically acceptable salt thereof.

In certain embodiments, the present invention provides a compound of formula I-1, I-1′, I-2, I-2′, I-3 or I-3′, wherein ERBM IS

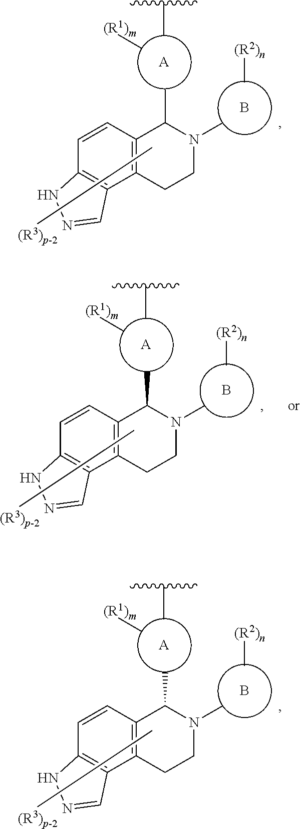

thereby forming a compound of one of formulas II-j-2, II-k-2, or II-1-2, respectively:

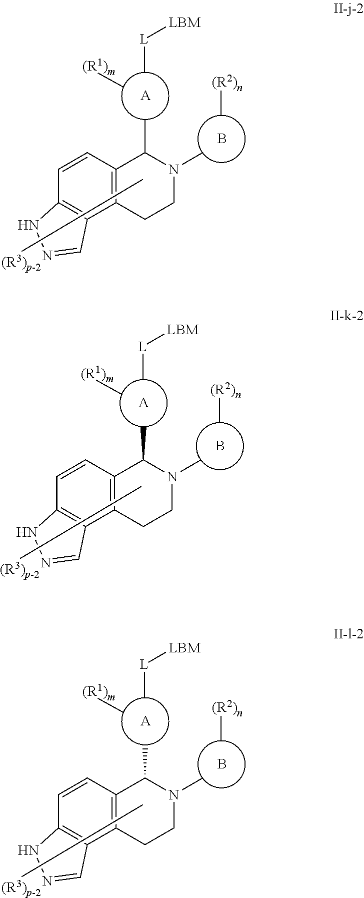

or a pharmaceutically acceptable salt thereof, wherein each of Ring A, Ring B, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM is

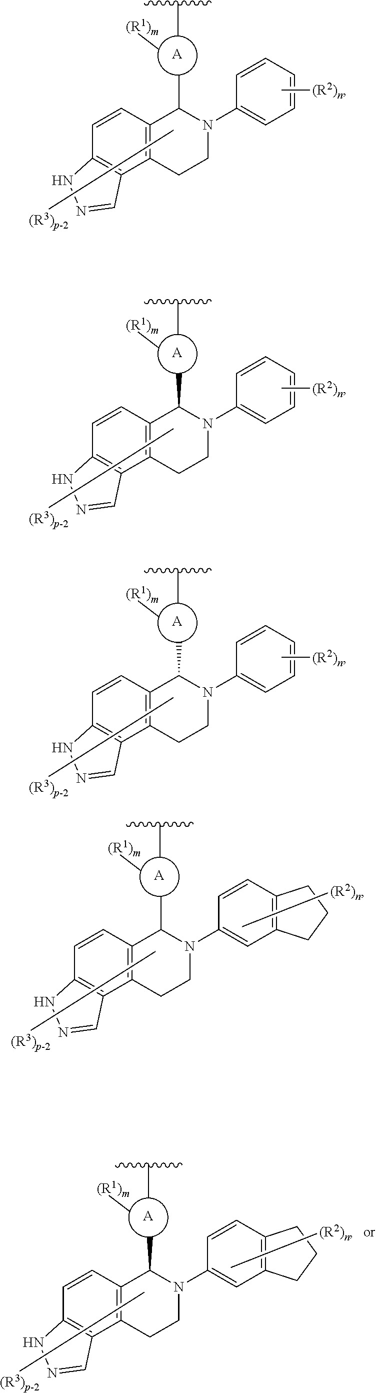

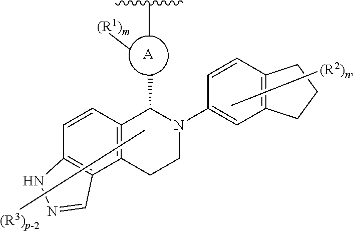

thereby forming a compound of one of formulas VII-a, VII-b, VII-c, VII-d, VII-e, or VII-f, respectively:

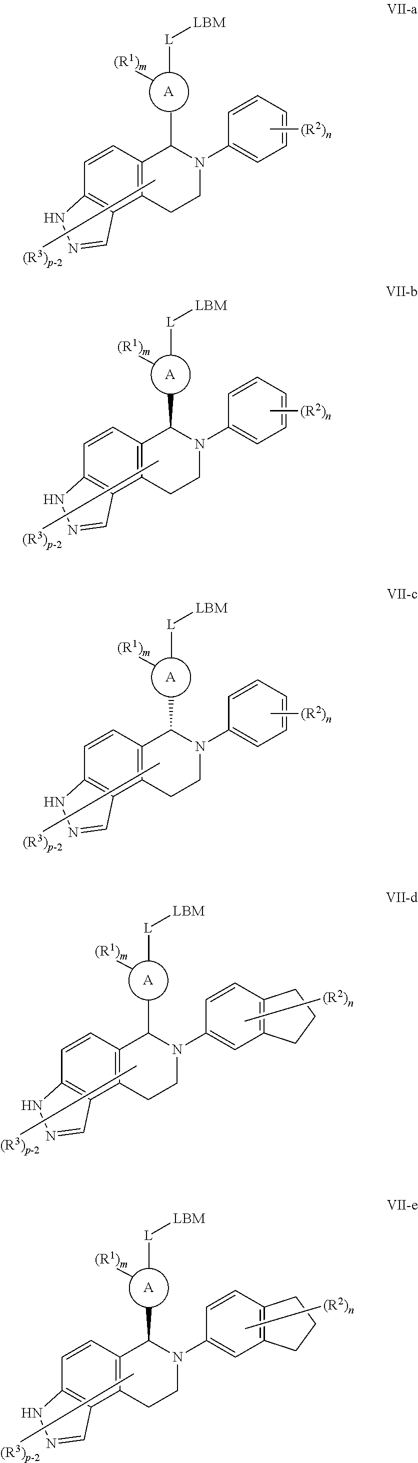

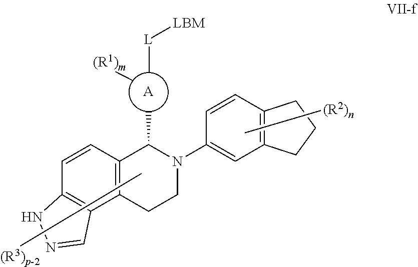

or a pharmaceutically acceptable salt thereof, wherein each of Ring A, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM is

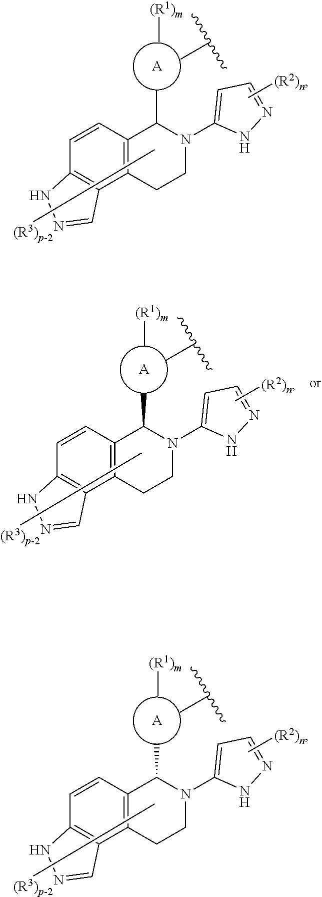

thereby forming a compound of one of formulas VII-g, VII-h, or VII-i, respectively:

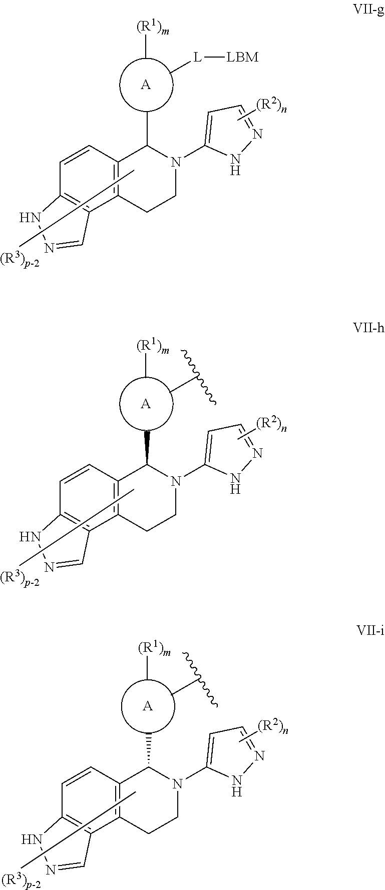

or a pharmaceutically acceptable salt thereof, wherein each of Ring A, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM is

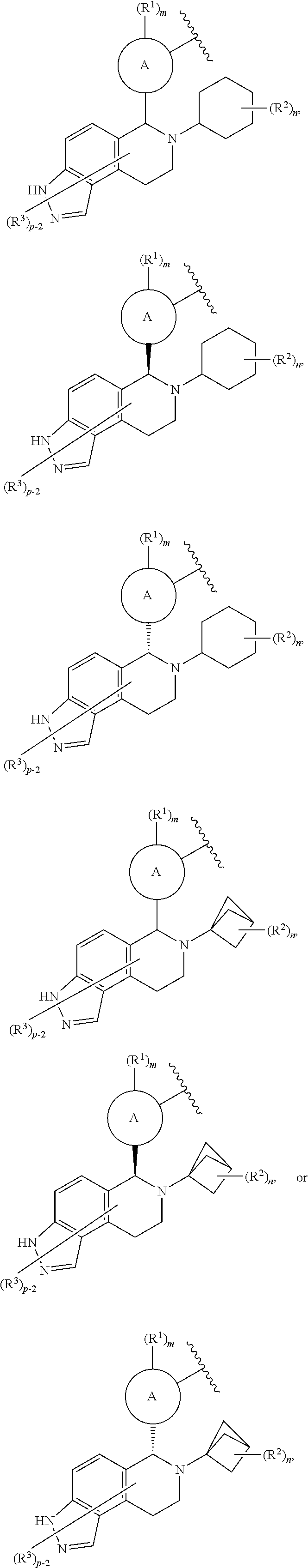

thereby forming a compound of one of formulas VII-j, VII-k, VII-l, VII-m, VII-n, or VII-o, respectively:

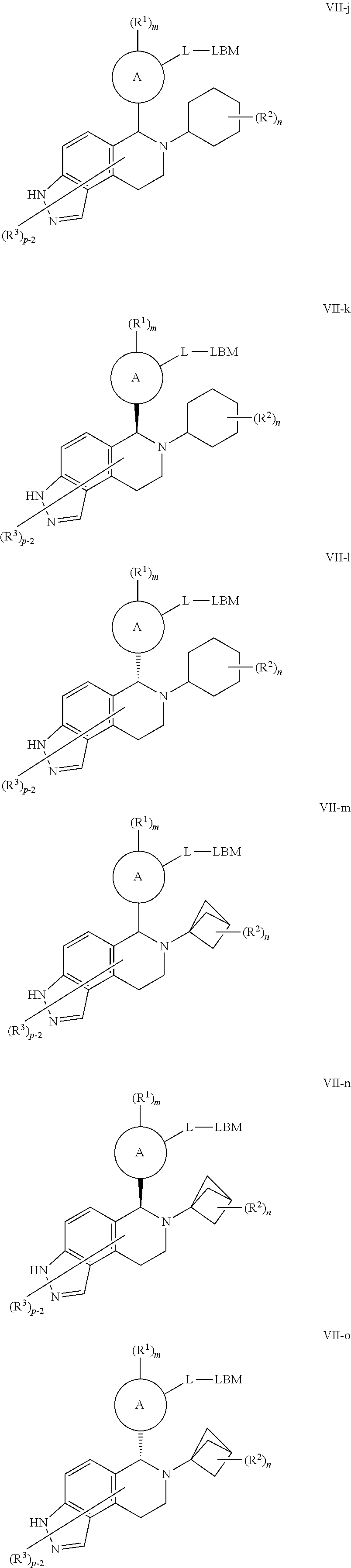

or a pharmaceutically acceptable salt thereof, wherein each of Ring A, R¹, R², R³, m, n, p, L, and LBM is as defined in embodiments and classes and subclasses herein.

In certain embodiments, the present invention provides a compound of formula I-1, I-1′, I-2, I-2′, I-3 or I-3′, wherein ERBM IS

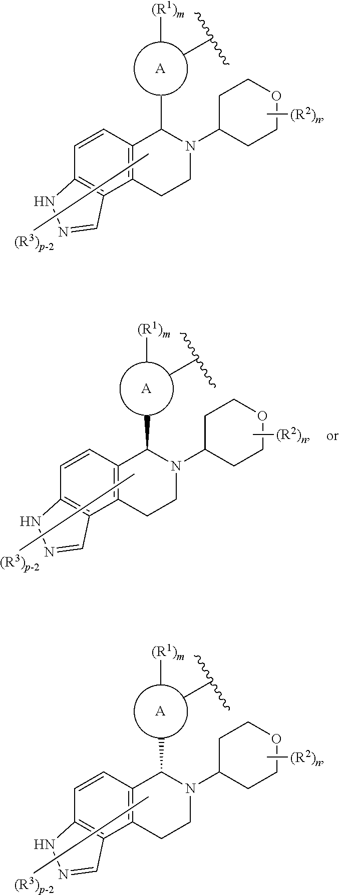

thereby forming a compound of one of formulas VII-p, VII-q, or VII-r, respectively:

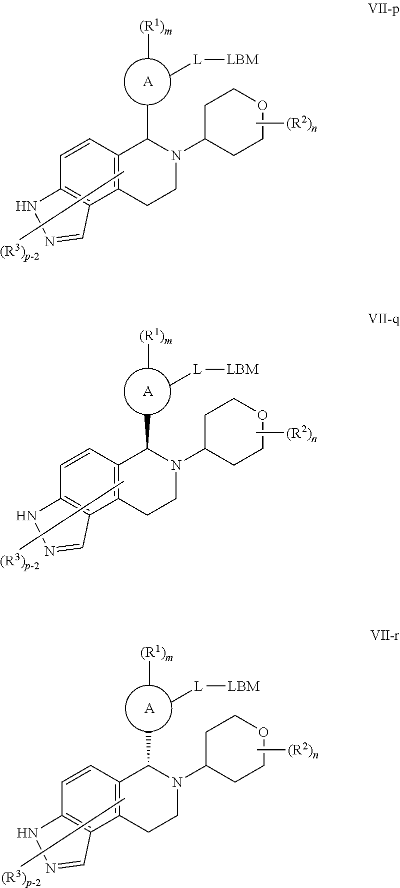

or a pharmaceutically acceptable salt thereof, wherein each of Ring A, R¹, R², R³, m, n, p, L, and LBM is as defined in embodiments and classes and subclasses herein.

In some embodiments, the compound is a compound of formula VII-a, or a pharmaceutically acceptable salt thereof.

In some embodiments, the compound is a compound of formula VII-b, or a pharmaceutically acceptable salt thereof.

In some embodiments, the compound is a compound of formula VII-c, or a pharmaceutically acceptable salt thereof.

In some embodiments, the compound is a compound of formula VII-d, or a pharmaceutically acceptable salt thereof.

In some embodiments, the compound is a compound of formula VII-e, or a pharmaceutically acceptable salt thereof.

In some embodiments, the compound is a compound of formula VII-f, or a pharmaceutically acceptable salt thereof.

In some embodiments, the compound is a compound of formula VII-g, or a pharmaceutically acceptable salt thereof.

In some embodiments, the compound is a compound of formula VII-h, or a pharmaceutically acceptable salt thereof.

In some embodiments, the compound is a compound of formula VII-i, or a pharmaceutically acceptable salt thereof.

In some embodiments, the compound is a compound of formula VII-j, or a pharmaceutically acceptable salt thereof.

In some embodiments, the compound is a compound of formula VII-k, or a pharmaceutically acceptable salt thereof.

In some embodiments, the compound is a compound of formula VII-l, or a pharmaceutically acceptable salt thereof.

In some embodiments, the compound is a compound of formula VII-m, or a pharmaceutically acceptable salt thereof.

In some embodiments, the compound is a compound of formula VII-n, or a pharmaceutically acceptable salt thereof.

In some embodiments, the compound is a compound of formula VII-o, or a pharmaceutically acceptable salt thereof.

In some embodiments, the compound is a compound of formula VII-p, or a pharmaceutically acceptable salt thereof.

In some embodiments, the compound is a compound of formula VII-q, or a pharmaceutically acceptable salt thereof.

In some embodiments, the compound is a compound of formula VII-r, or a pharmaceutically acceptable salt thereof.

In some embodiments, the compound is a compound of one of formulas VIII-d, VIII-e, and VIII-f:

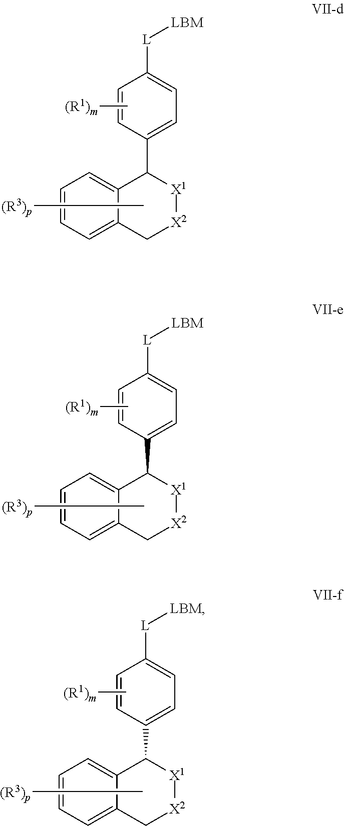

or a pharmaceutically acceptable salt thereof wherein each of X¹, X², R¹, R³, m, p, L, and LBM is as defined in embodiments and classes and subclasses herein. In some embodiments, the compound is a compound of formula VIII-d, or a pharmaceutically acceptable salt thereof.

In some embodiments, the compound is a compound of formula VIII-e, or a pharmaceutically acceptable salt thereof.

In some embodiments, the compound is a compound of formula VIII-f, or a pharmaceutically acceptable salt thereof.

In some embodiments, the compound is a compound of one of formulas VIII-g, VIII-h, VIII-i, VIII-j, VIII-k or VIII-l:

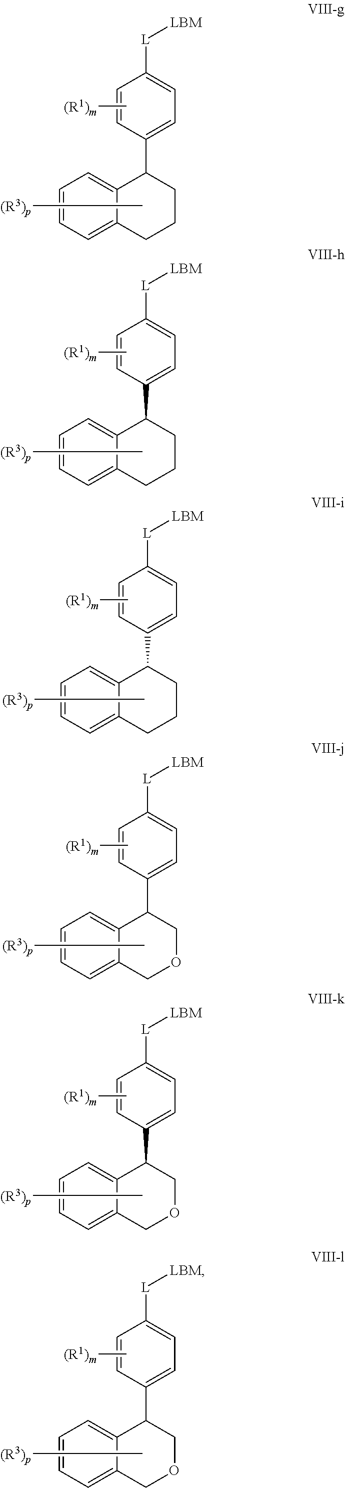

or a pharmaceutically acceptable salt thereof wherein each of Ring A, R¹, R³, m, p, L, and LBM is as defined in embodiments and classes and subclasses herein.

In some embodiments, the compound is a compound of formula VIII-g, or a pharmaceutically acceptable salt thereof.

In some embodiments, the compound is a compound of formula VIII-h, or a pharmaceutically acceptable salt thereof.

In some embodiments, the compound is a compound of formula VIII-i, or a pharmaceutically acceptable salt thereof.

In some embodiments, the compound is a compound of formula VIII-j, or a pharmaceutically acceptable salt thereof.

In some embodiments, the compound is a compound of formula VIII-k, or a pharmaceutically acceptable salt thereof.

In some embodiments, the compound is a compound of formula VIII-l, or a pharmaceutically acceptable salt thereof.

In some embodiments, the compound is a compound of one of formulas VIII-m, VIII-n, VIII-o, VIII-p, VIII-q, VIII-r, VIII-s, VIII-t, or VIII-u:

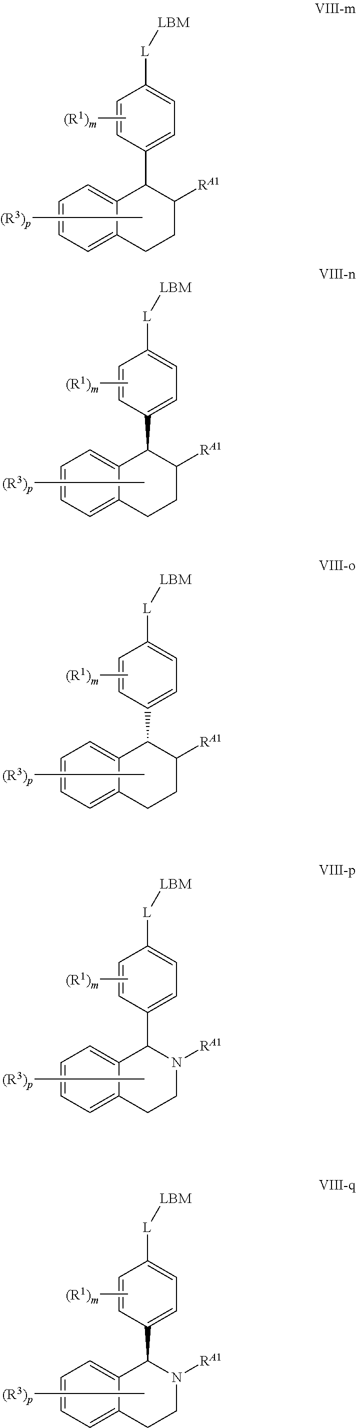

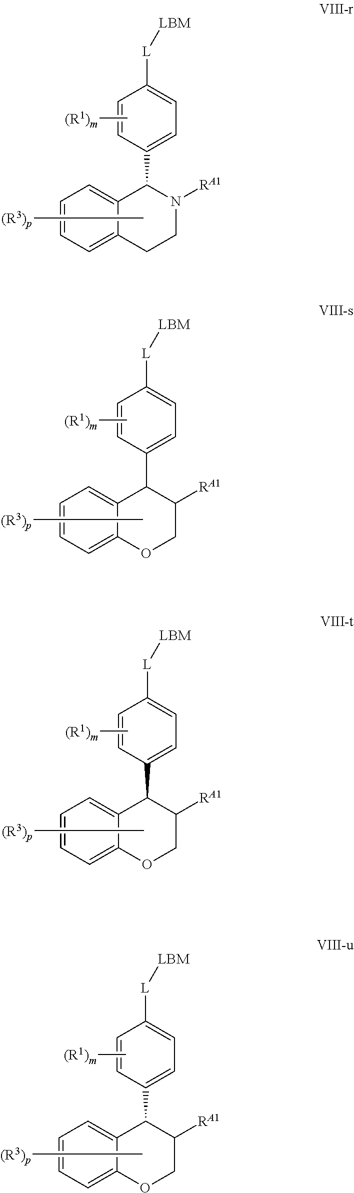

or a pharmaceutically acceptable salt thereof wherein each of R^{A1}, R¹, R³, m, p, L, and LBM is as defined in embodiments and classes and subclasses herein.

In some embodiments, the compound is a compound of formula VIII-m, or a pharmaceutically acceptable salt thereof.

In some embodiments, the compound is a compound of formula VIII-n, or a pharmaceutically acceptable salt thereof.

In some embodiments, the compound is a compound of formula VIII-o, or a pharmaceutically acceptable salt thereof.

In some embodiments, the compound is a compound of formula VIII-p, or a pharmaceutically acceptable salt thereof.

In some embodiments, the compound is a compound of formula VIII-q, or a pharmaceutically acceptable salt thereof.

In some embodiments, the compound is a compound of formula VIII-r, or a pharmaceutically acceptable salt thereof.

In some embodiments, the compound is a compound of formula VIII-s, or a pharmaceutically acceptable salt thereof.

In some embodiments, the compound is a compound of formula VIII-t, or a pharmaceutically acceptable salt thereof.

In some embodiments, the compound is a compound of formula VIII-u, or a pharmaceutically acceptable salt thereof.

In some embodiments, the compound is a compound of one of formulas IX-d, IX-e, IX-f:

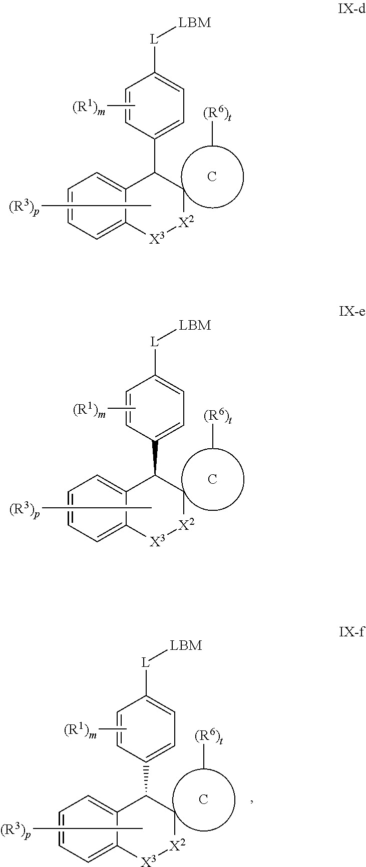

or a pharmaceutically acceptable salt thereof wherein each of X¹, X², Ring C, R¹, R³, R⁶, m, p, t, L, and LBM is as defined in embodiments and classes and subclasses herein.

In some embodiments, the compound is a compound of formula IX-d, or a pharmaceutically acceptable salt thereof.

In some embodiments, the compound is a compound of formula IX-e, or a pharmaceutically acceptable salt thereof.

In some embodiments, the compound is a compound of formula IX-f, or a pharmaceutically acceptable salt thereof.

In some embodiments, the compound is a compound of one of formulas IX-g, IX-h, IX-i, IX-j, IX-k, IX-l, IX-m, IX-n, IX-o:

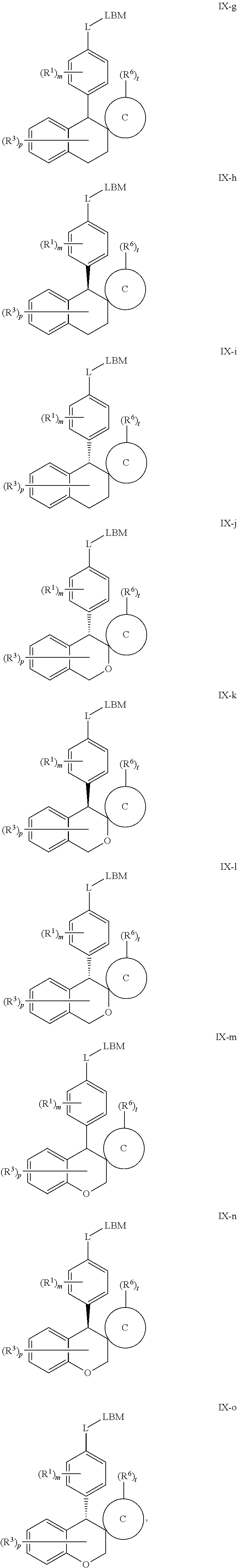

or a pharmaceutically acceptable salt thereof, wherein each of X¹, X², X³, Ring A, Ring C, R¹, R³, R⁶, m, p, t, L, and LBM is as defined in embodiments and classes and subclasses herein.

In some embodiments, the compound is a compound of formula IX-g, or a pharmaceutically acceptable salt thereof.

In some embodiments, the compound is a compound of formula IX-h, or a pharmaceutically acceptable salt thereof.

In some embodiments, the compound is a compound of formula IX-i, or a pharmaceutically acceptable salt thereof.

In some embodiments, the compound is a compound of formula IX-j, or a pharmaceutically acceptable salt thereof.

In some embodiments, the compound is a compound of formula IX-k, or a pharmaceutically acceptable salt thereof.

In some embodiments, the compound is a compound of formula IX-l, or a pharmaceutically acceptable salt thereof.

In some embodiments, the compound is a compound of formula IX-m, or a pharmaceutically acceptable salt thereof.

In some embodiments, the compound is a compound of formula IX-n, or a pharmaceutically acceptable salt thereof.

In some embodiments, the compound is a compound of formula IX-o, or a pharmaceutically acceptable salt thereof.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1 or Table 2, below, or a pharmaceutically acceptable salt, stereoisomer, or mixture of stereoisomers thereof. In some embodiments, the present disclosure provides a compound set forth in Table 1 or Table 2, below, or a pharmaceutically acceptable salt thereof. In some embodiments, the present disclosure provides a compound set forth in Table 1 or Table 2, below.

TABLE 1
Representative Compounds of the Disclosure

| Compound | |
|---|---|
| No. | Structure |
| I-1 | 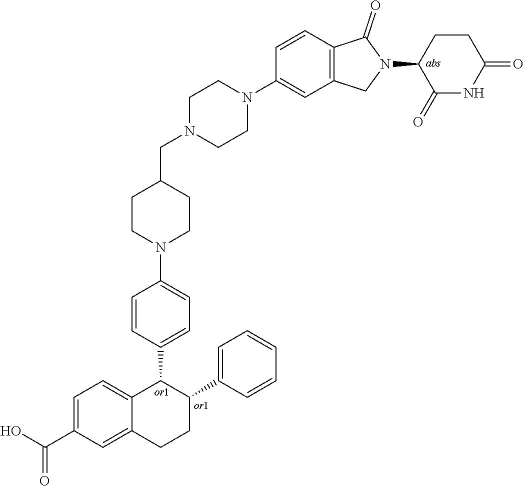 [SMILES: O=C1CC[C@H](N2Cc3cc(N4CCN(CC5CCN(c6ccc([C@H]7c8ccc(C(=O)O)cc8CC[C@H]7c7ccccc7)cc6)CC5)CC4)ccc3C2=O)C(=O)N1] |
| I-2 | 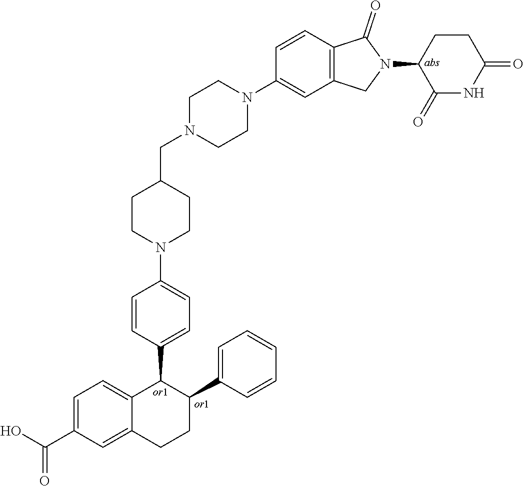 [SMILES: O=C1CC[C@H](N2Cc3cc(N4CCN(CC5CCN(c6ccc([C@@H]7c8ccc(C(=O)O)cc8CC[C@@H]7c7ccccc7)cc6)CC5)CC4)ccc3C2=O)C(=O)N1] |
| I-3 | 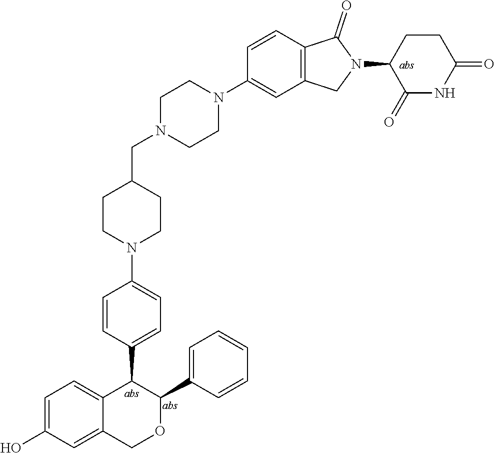 [SMILES: O=C1CC[C@H](N2Cc3cc(N4CCN(CC5CCN(c6ccc([C@@H]7c8ccc(O)cc8CO[C@@H]7c7ccccc7)cc6)CC5)CC4)ccc3C2=O)C(=O)N1] |
| I-4 | 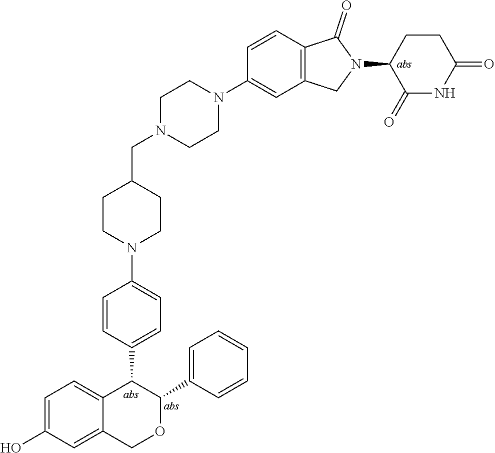 [SMILES: O=C1CC[C@H](N2Cc3cc(N4CCN(CC5CCN(c6ccc([C@H]7c8ccc(O)cc8CO[C@H]7c7ccccc7)cc6)CC5)CC4)ccc3C2=O)C(=O)N1] |
| I-5 | 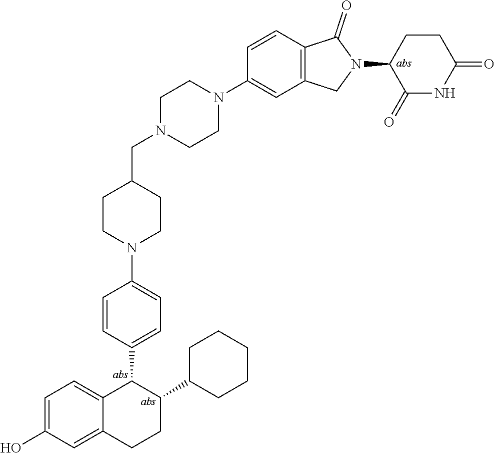 [SMILES: O=C1CC[C@H](N2Cc3cc(N4CCN(CC5CCN(c6ccc([C@H]7c8ccc(O)cc8CC[C@H]7C7CCCCC7)cc6)CC5)CC4)ccc3C2=O)C(=O)N1] |
| I-6 | 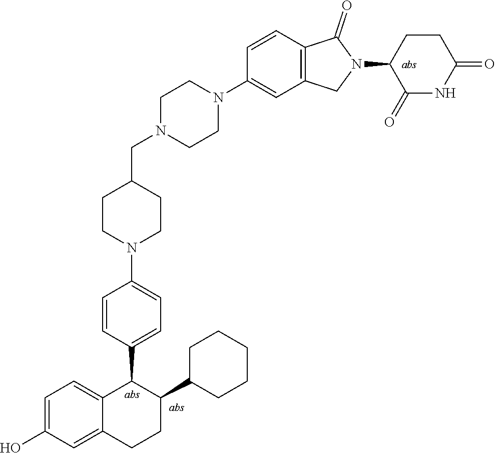 [SMILES: O=C1CC[C@H](N2Cc3cc(N4CCN(CC5CCN(c6ccc([C@@H]7c8ccc(O)cc8CC[C@@H]7C7CCCCC7)cc6)CC5)CC4)ccc3C2=O)C(=O)N1] |
| I-7 | 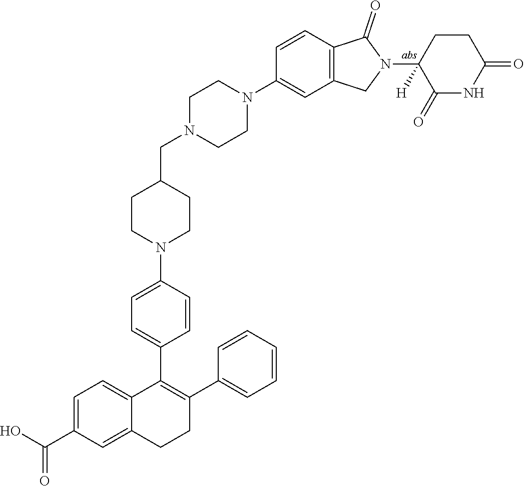 [SMILES: O=C1CC[C@H](N2Cc3cc(N4CCN(CC5CCN(c6ccc(C7=C(c8ccccc8)CCc8cc(C(=O)O)ccc87)cc6)CC5)CC4)ccc3C2=O)C(=O)N1] |
| I-8 | 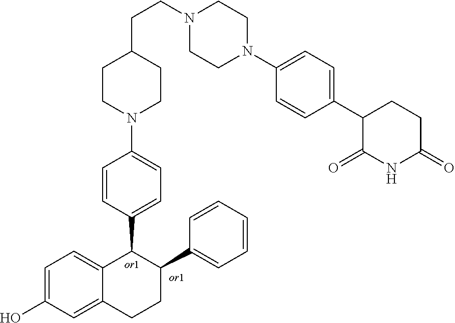 [SMILES: O=C1CCC(c2ccc(N3CCN(CCC4CCN(c5ccc([C@@H]6c7ccc(O)cc7CC[C@@H]6c6ccccc6)cc5)CC4)CC3)cc2)C(=O)N1] |
| I-9 | 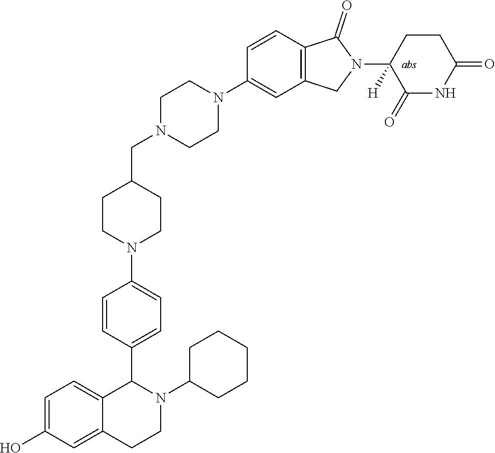 [SMILES: O=C1CC[C@H](N2Cc3cc(N4CCN(CC5CCN(c6ccc(C7c8ccc(O)cc8CCN7C7CCCCC7)cc6)CC5)CC4)ccc3C2=O)C(=O)N1] |
| I-10 | 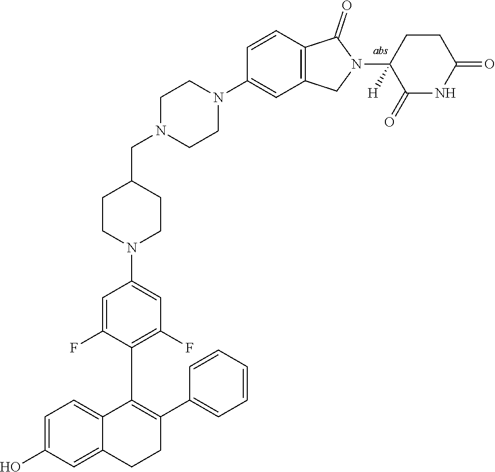 [SMILES: O=C1CC[C@H](N2Cc3cc(N4CCN(CC5CCN(c6cc(F)c(C7=C(c8ccccc8)CCc8cc(O)ccc87)c(F)c6)CC5)CC4)ccc3C2=O)C(=O)N1] |
| I-11‡ | 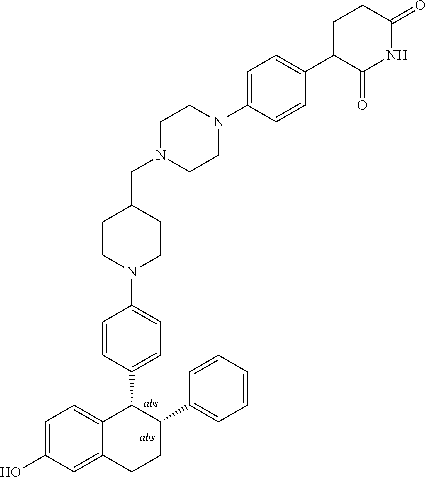 [SMILES: O=C1CCC(c2ccc(N3CCN(CC4CCN(c5ccc([C@H]6c7ccc(O)cc7CC[C@H]6c6ccccc6)cc5)CC4)CC3)cc2)C(=O)N1] |
| I-12 | 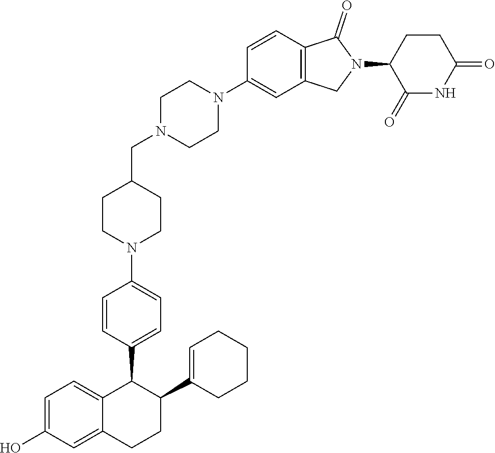 [SMILES: O=C1CC[C@H](N2Cc3cc(N4CCN(CC5CCN(c6ccc([C@@H]7c8ccc(O)cc8CC[C@@H]7C7=CCCCC7)cc6)CC5)CC4)ccc3C2=O)C(=O)N1] |
| I-13‡ | 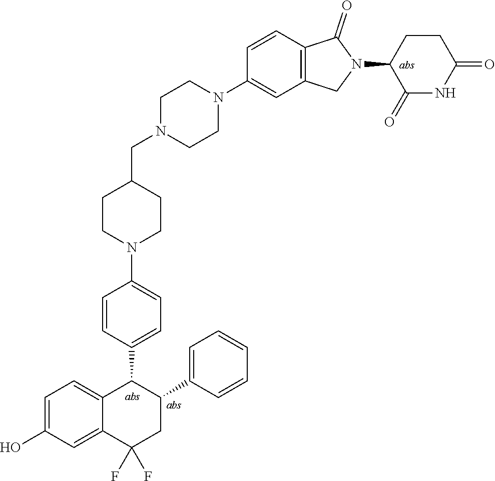 [SMILES: O=C1CC[C@H](N2Cc3cc(N4CCN(CC5CCN(c6ccc([C@H]7c8ccc(O)cc8C(F)(F)C[C@H]7c7ccccc7)cc6)CC5)CC4)ccc3C2=O)C(=O)N1] |
| I-14 | 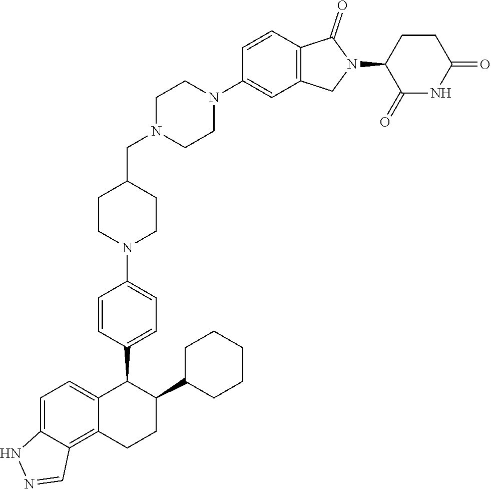 [SMILES: O=C1CC[C@H](N2Cc3cc(N4CCN(CC5CCN(c6ccc([C@@H]7c8ccc9[nH]ncc9c8CC[C@@H]7C7CCCCC7)cc6)CC5)CC4)ccc3C2=O)C(=O)N1] |
| I-15(*) | 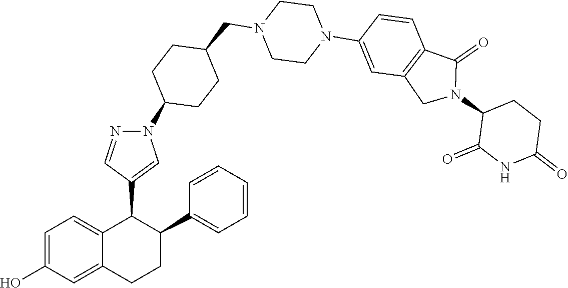 [SMILES: O=C1CC[C@H](N2Cc3cc(N4CCN(C[C@H]5CC[C@@H](n6cc([C@@H]7c8ccc(O)cc8CC[C@@H]7c7ccccc7)cn6)CC5)CC4)ccc3C2=O)C(=O)N1] |
| I-16(*) | 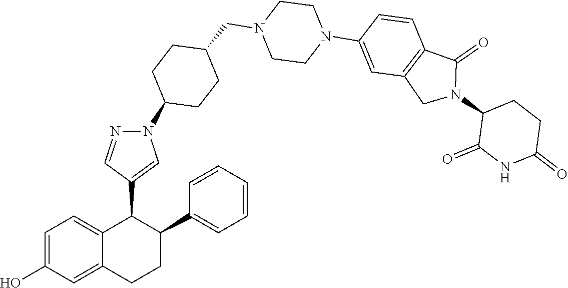 [SMILES: O=C1CC[C@H](N2Cc3cc(N4CCN(C[C@H]5CC[C@H](n6cc([C@@H]7c8ccc(O)cc8CC[C@@H]7c7ccccc7)cn6)CC5)CC4)ccc3C2=O)C(=O)N1] |
| I-17 | 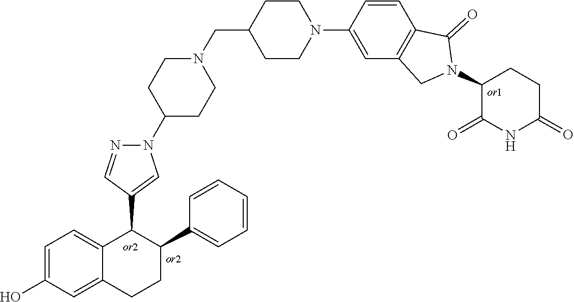 [SMILES: O=C1CC[C@H](N2Cc3cc(N4CCC(CN5CCC(n6cc([C@@H]7c8ccc(O)cc8CC[C@@H]7c7ccccc7)cn6)CC5)CC4)ccc3C2=O)C(=O)N1] |
| I-18(*) | 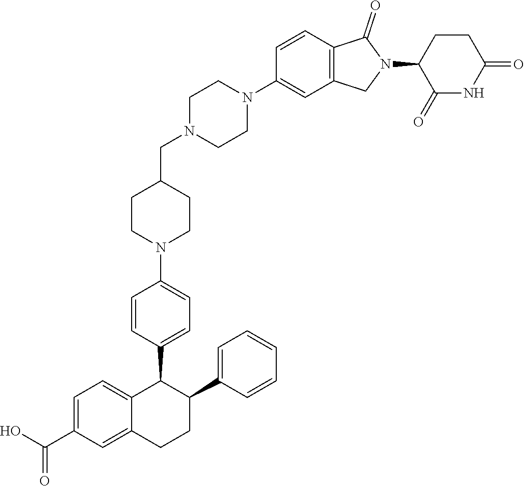 [SMILES: O=C1CC[C@H](N2Cc3cc(N4CCN(CC5CCN(c6ccc([C@@H]7c8ccc(C(=O)O)cc8CC[C@@H]7c7ccccc7)cc6)CC5)CC4)ccc3C2=O)C(=O)N1] |
| I-19(*) | 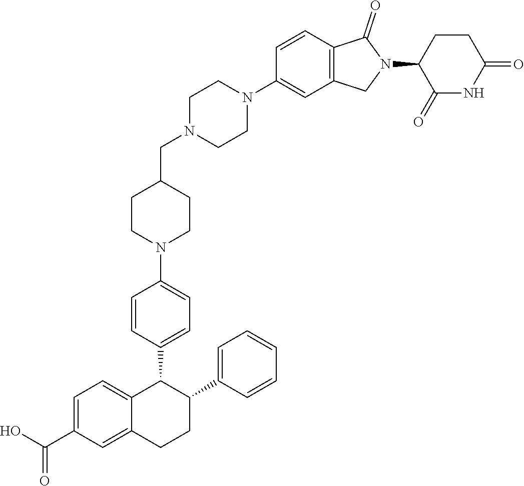 [SMILES: O=C1CC[C@H](N2Cc3cc(N4CCN(CC5CCN(c6ccc([C@H]7c8ccc(C(=O)O)cc8CC[C@H]7c7ccccc7)cc6)CC5)CC4)ccc3C2=O)C(=O)N1] |
| I-20 | 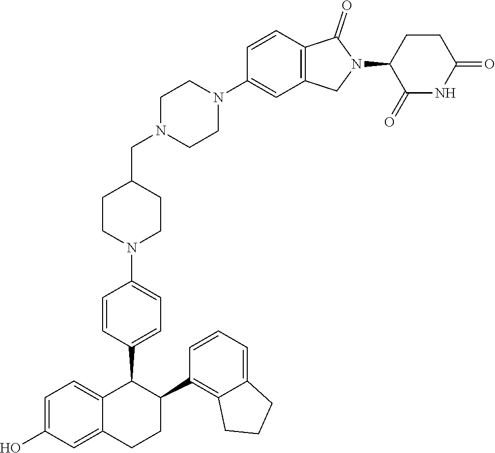 [SMILES: O=C1CC[C@H](N2Cc3cc(N4CCN(CC5CCN(c6ccc([C@@H]7c8ccc(O)cc8CC[C@@H]7c7cccc8c7CCC8)cc6)CC5)CC4)ccc3C2=O)C(=O)N1] |
| I-21 | 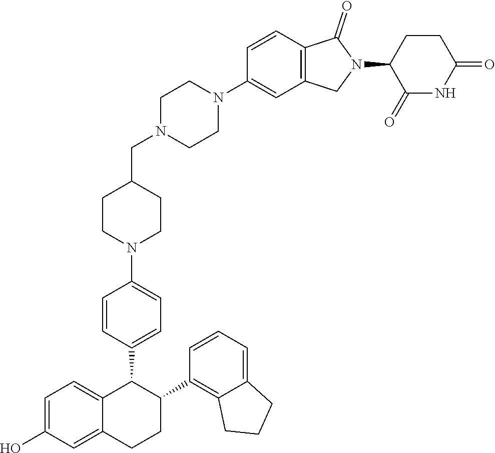 [SMILES: O=C1CC[C@H](N2Cc3cc(N4CCN(CC5CCN(c6ccc([C@H]7c8ccc(O)cc8CC[C@H]7c7cccc8c7CCC8)cc6)CC5)CC4)ccc3C2=O)C(=O)N1] |
| I-22 | 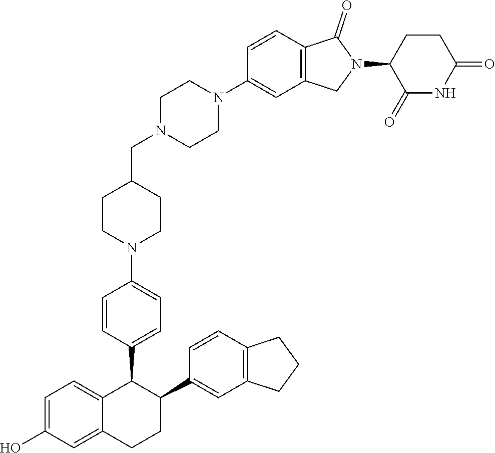 [SMILES: O=C1CC[C@H](N2Cc3cc(N4CCN(CC5CCN(c6ccc([C@@H]7c8ccc(O)cc8CC[C@@H]7c7ccc8c(c7)CCC8)cc6)CC5)CC4)ccc3C2=O)C(=O)N1] |
| I-23 | 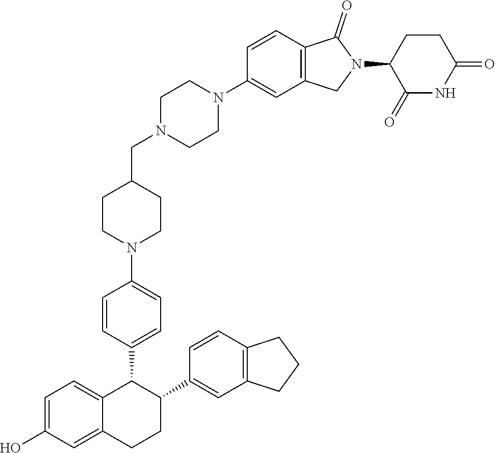 [SMILES: O=C1CC[C@H](N2Cc3cc(N4CCN(CC5CCN(c6ccc([C@H]7c8ccc(O)cc8CC[C@H]7c7ccc8c(c7)CCC8)cc6)CC5)CC4)ccc3C2=O)C(=O)N1] |
| I-24 | 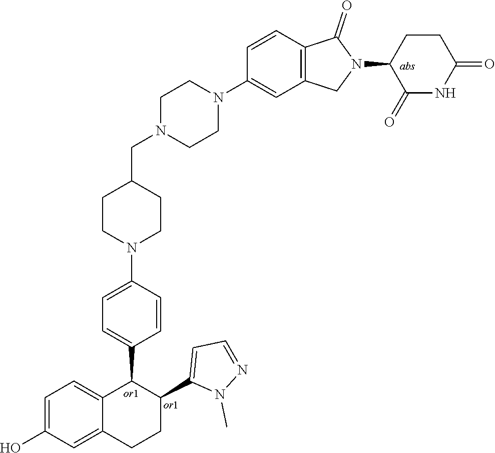 [SMILES: Cn1nccc1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-25 | 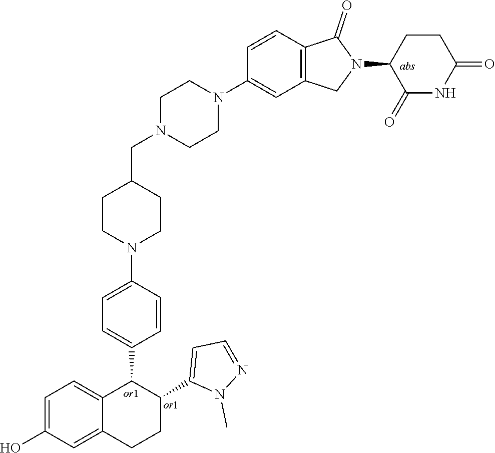 [SMILES: Cn1nccc1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-26 | 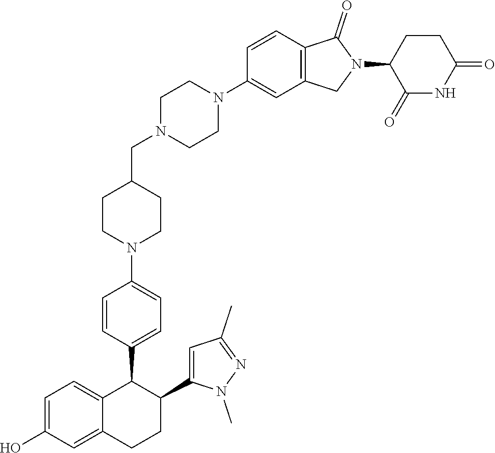 [SMILES: Cc1cc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)n(C)n1] |
| I-27 | 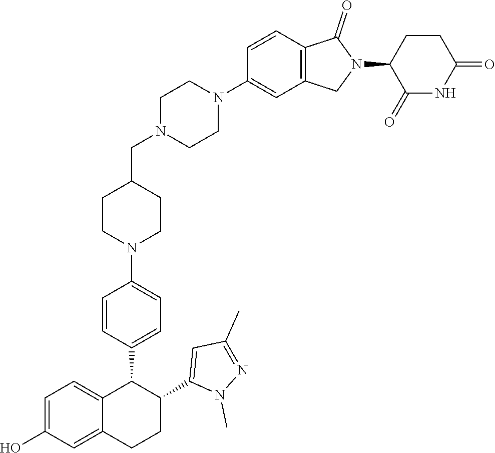 [SMILES: Cc1cc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n(C)n1] |
| I-28 | 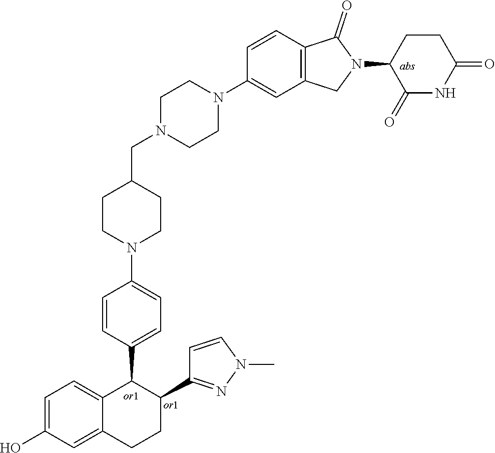 [SMILES: Cn1ccc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)n1] |
| I-29 | 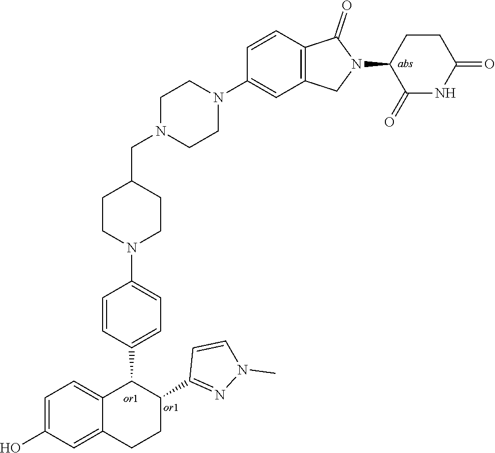 [SMILES: Cn1ccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n1] |
| I-30‡ | 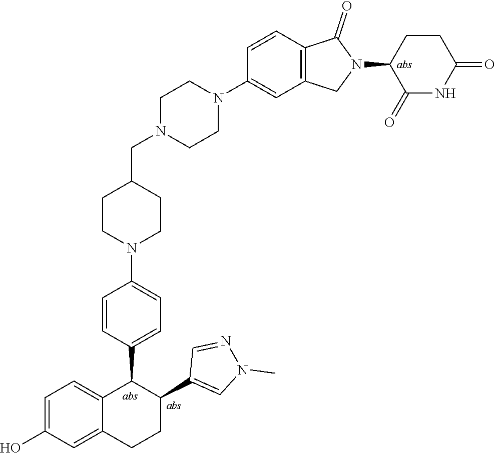 [SMILES: Cn1cc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)cn1] |
| I-31‡ | 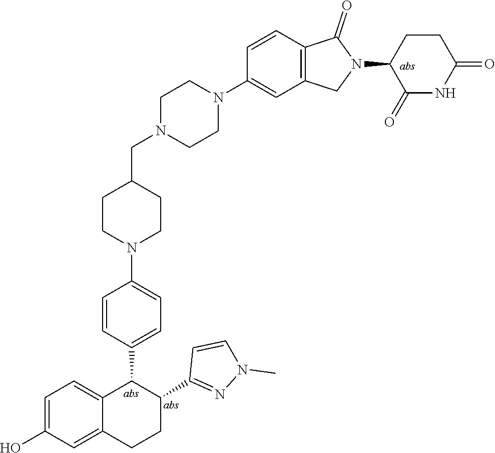 [SMILES: Cn1ccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n1] |
| I-32 | 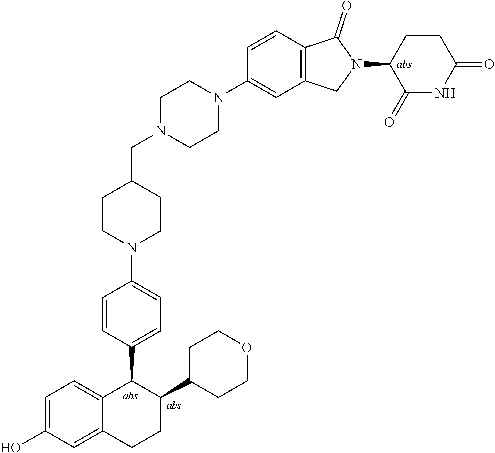 [SMILES: O=C1CC[C@H](N2Cc3cc(N4CCN(CC5CCN(c6ccc([C@@H]7c8ccc(O)cc8CC[C@@H]7C7CCOCC7)cc6)CC5)CC4)ccc3C2=O)C(=O)N1] |
| I-33 | 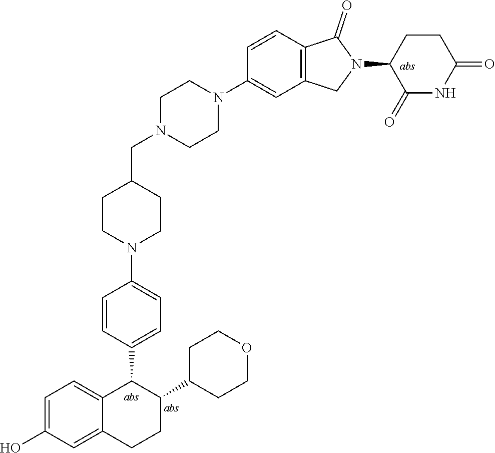 [SMILES: O=C1CC[C@H](N2Cc3cc(N4CCN(CC5CCN(c6ccc([C@H]7c8ccc(O)cc8CC[C@H]7C7CCOCC7)cc6)CC5)CC4)ccc3C2=O)C(=O)N1] |
| I-34 | 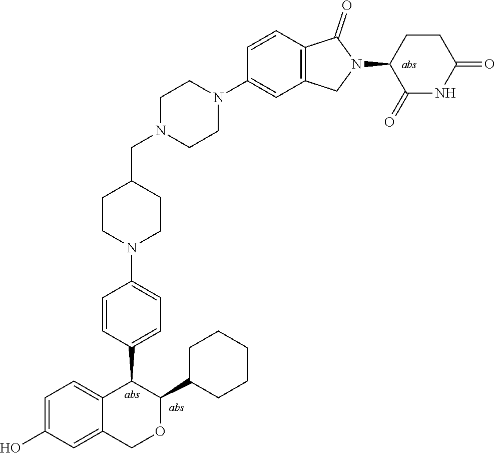 [SMILES: O=C1CC[C@H](N2Cc3cc(N4CCN(CC5CCN(c6ccc([C@@H]7c8ccc(O)cc8CO[C@@H]7C7CCCCC7)cc6)CC5)CC4)ccc3C2=O)C(=O)N1] |
| I-35 | 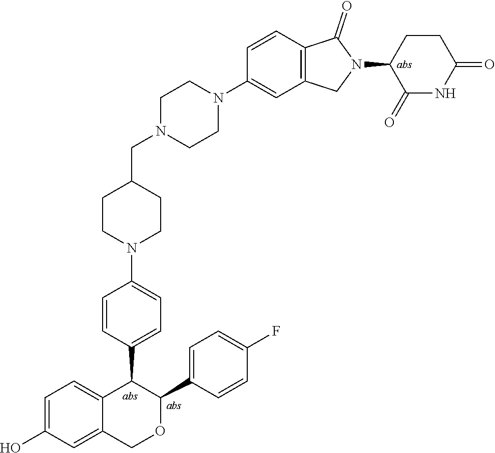 [SMILES: O=C1CC[C@H](N2Cc3cc(N4CCN(CC5CCN(c6ccc([C@@H]7c8ccc(O)cc8CO[C@@H]7c7ccc(F)cc7)cc6)CC5)CC4)ccc3C2=O)C(=O)N1] |
| I-36 | 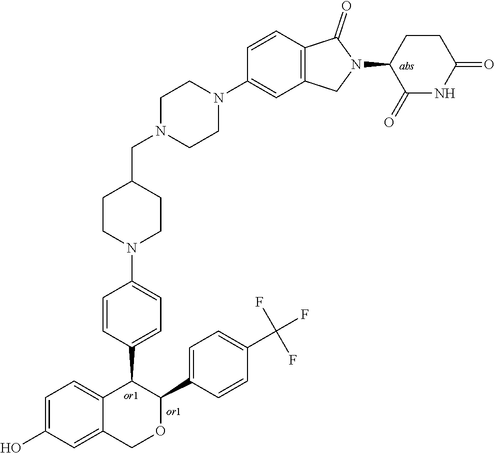 [SMILES: O=C1CC[C@H](N2Cc3cc(N4CCN(CC5CCN(c6ccc([C@@H]7c8ccc(O)cc8CO[C@@H]7c7ccc(C(F)(F)F)cc7)cc6)CC5)CC4)ccc3C2=O)C(=O)N1] |
| I-37(*) | 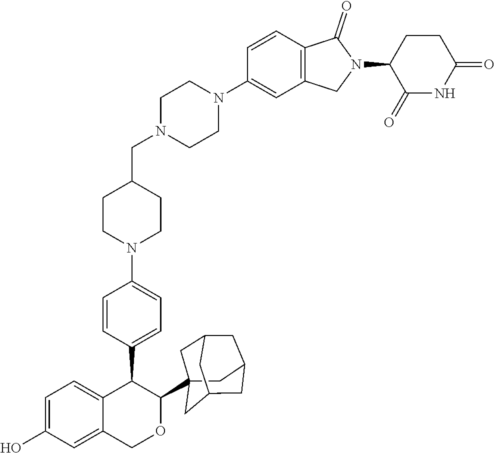 [SMILES: O=C1CC[C@H](N2Cc3cc(N4CCN(CC5CCN(c6ccc([C@@H]7c8ccc(O)cc8CO[C@@H]7C78CC9CC(CC(C9)C7)C8)cc6)CC5)CC4)ccc3C2=O)C(=O)N1] |
| I-38(*) | 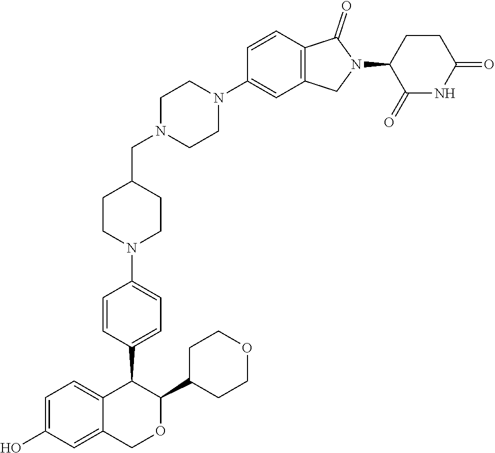 [SMILES: O=C1CC[C@H](N2Cc3cc(N4CCN(CC5CCN(c6ccc([C@@H]7c8ccc(O)cc8CO[C@@H]7C7CCOCC7)cc6)CC5)CC4)ccc3C2=O)C(=O)N1] |
| I-39‡ | 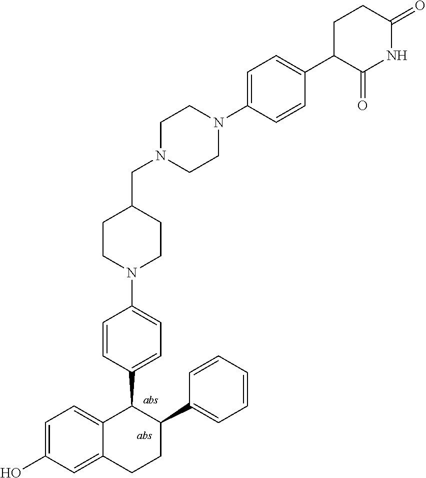 [SMILES: O=C1CCC(c2ccc(N3CCN(CC4CCN(c5ccc([C@@H]6c7ccc(O)cc7CC[C@@H]6c6ccccc6)cc5)CC4)CC3)cc2)C(=O)N1] |
| I-40 | 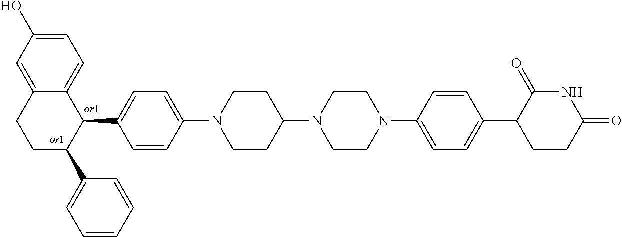 [SMILES: O=C1CCC(c2ccc(N3CCN(C4CCN(c5ccc([C@@H]6c7ccc(O)cc7CC[C@@H]6c6ccccc6)cc5)CC4)CC3)cc2)C(=O)N1] |
| I-41 | 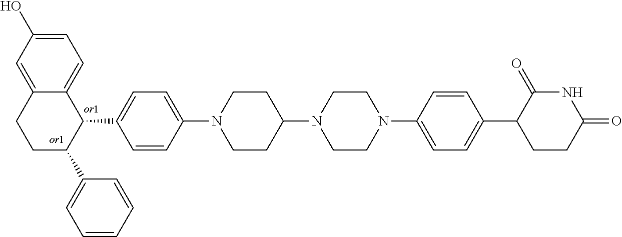 [SMILES: O=C1CCC(c2ccc(N3CCN(C4CCN(c5ccc([C@H]6c7ccc(O)cc7CC[C@H]6c6ccccc6)cc5)CC4)CC3)cc2)C(=O)N1] |
| I-42 | 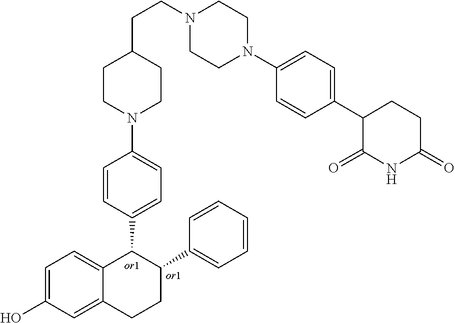 [SMILES: O=C1CCC(c2ccc(N3CCN(CCC4CCN(c5ccc([C@H]6c7ccc(O)cc7CC[C@H]6c6ccccc6)cc5)CC4)CC3)cc2)C(=O)N1] |
| I-43 | 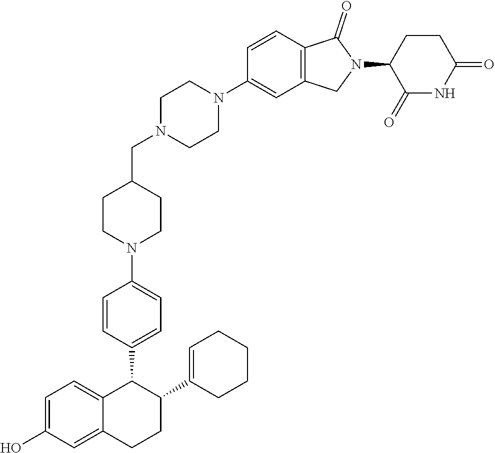 [SMILES: O=C1CC[C@H](N2Cc3cc(N4CCN(CC5CCN(c6ccc([C@H]7c8ccc(O)cc8CC[C@H]7C7=CCCCC7)cc6)CC5)CC4)ccc3C2=O)C(=O)N1] |
| I-44 | 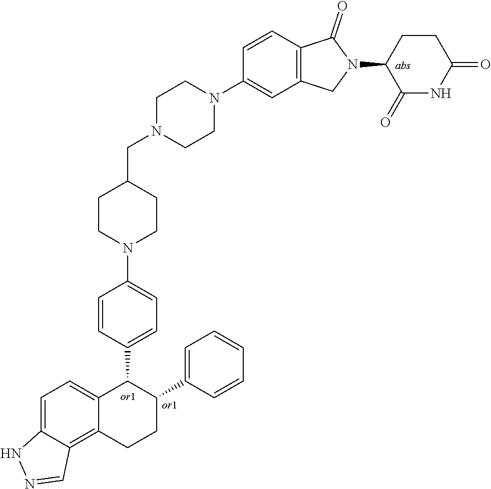 [SMILES: O=C1CC[C@H](N2Cc3cc(N4CCN(CC5CCN(c6ccc([C@H]7c8ccc9[nH]ncc9c8CC[C@H]7c7ccccc7)cc6)CC5)CC4)ccc3C2=O)C(=O)N1] |
| I-45 | 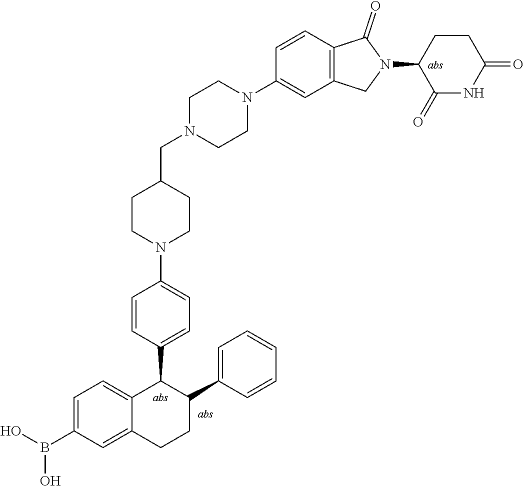 |
| I-46 | 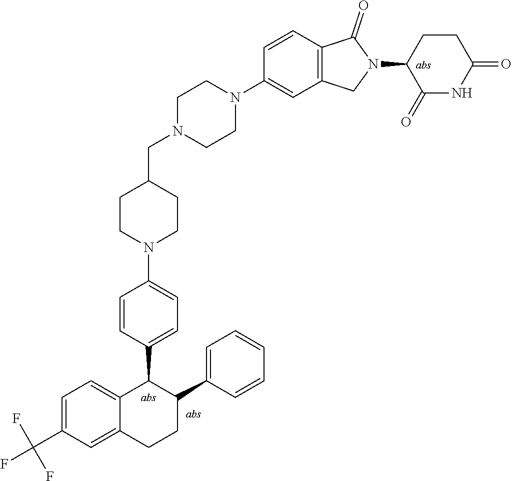 |
| I-47 | 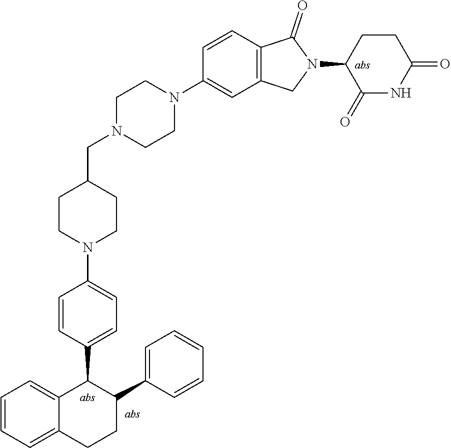 |
| I-48 | 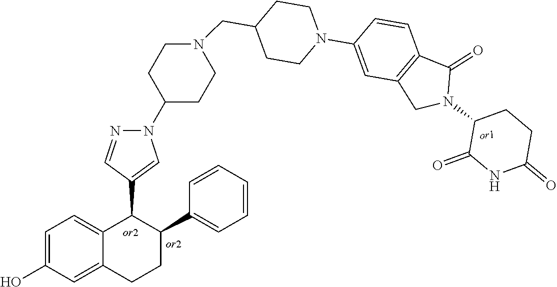 [SMILES: O=C1CC[C@@H](N2Cc3cc(N4CCC(CN5CCC(n6cc([C@@H]7c8ccc(O)cc8CC[C@@H]7c7ccccc7)cn6)CC5)CC4)ccc3C2=O)C(=O)N1] |
| I-49 | 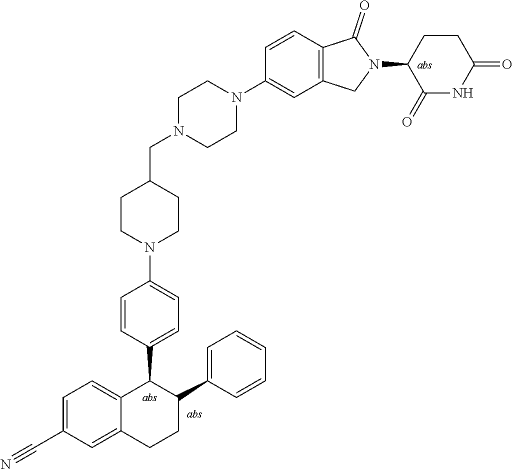 |
| I-50 | 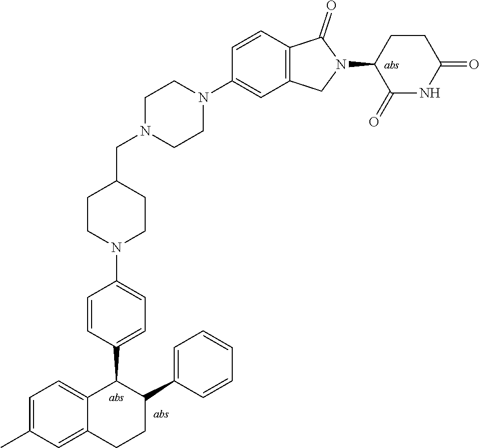 |
| I-51‡ | 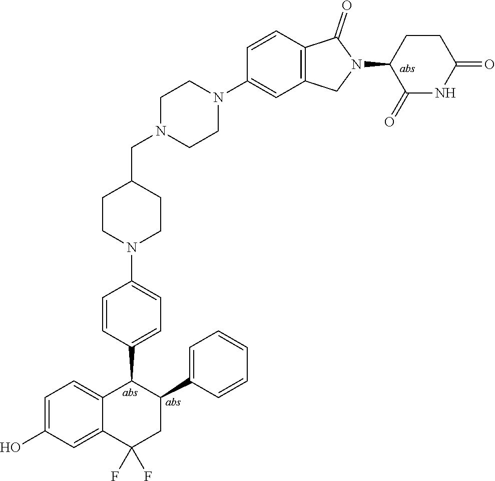 [SMILES: O=C1CC[C@H](N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1] |
| I-52 | 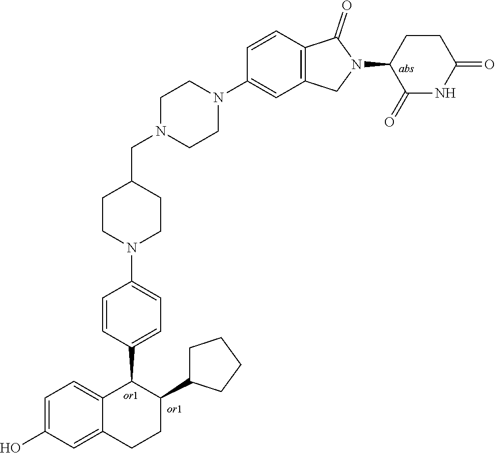 [SMILES: O=C1CC[C@H](N2Cc3cc(N4CCN(CC5CCN(c6ccc([C@@H]7c8ccc(O)cc8CC[C@@H]7C7CCCC7)cc6)CC5)CC4)ccc3C2=O)C(=O)N1] |
| I-53 | 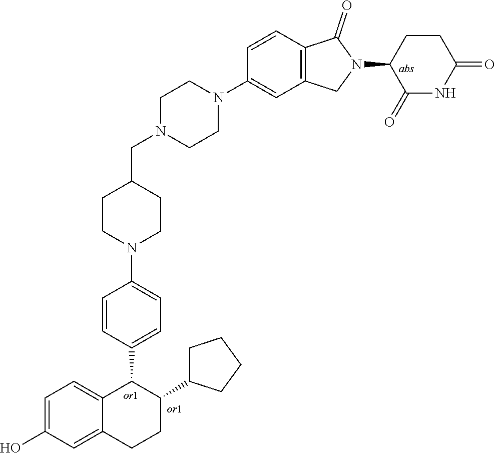 [SMILES: O=C1CC[C@H](N2Cc3cc(N4CCN(CC5CCN(c6ccc([C@H]7c8ccc(O)cc8CC[C@H]7C7CCCC7)cc6)CC5)CC4)ccc3C2=O)C(=O)N1] |
| I-54 | 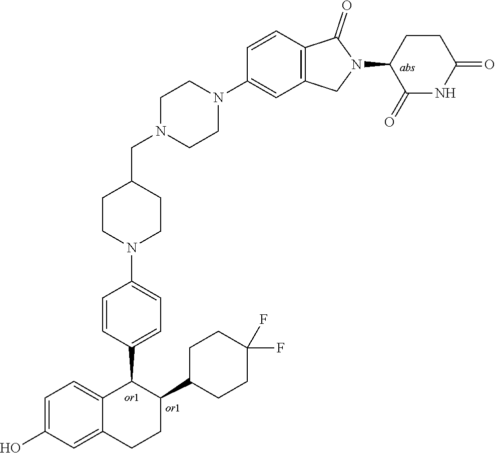 [SMILES: O=C1CC[C@H](N2Cc3cc(N4CCN(CC5CCN(c6ccc([C@@H]7c8ccc(O)cc8CC[C@@H]7C7CCC(F)(F)CC7)cc6)CC5)CC4)ccc3C2=O)C(=O)N1] |
| I-55 | 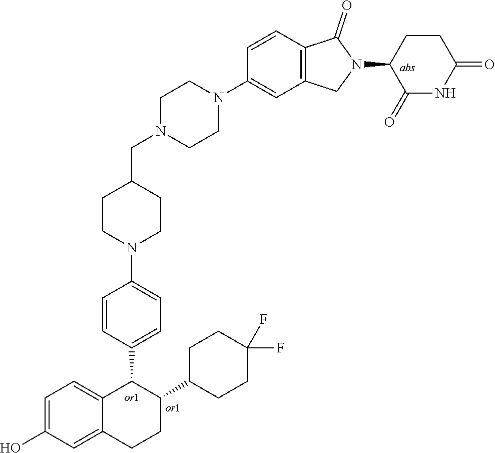 [SMILES: O=C1CC[C@H](N2Cc3cc(N4CCN(CC5CCN(c6ccc([C@H]7c8ccc(O)cc8CC[C@H]7C7CCC(F)(F)CC7)cc6)CC5)CC4)ccc3C2=O)C(=O)N1] |
| I-56 | 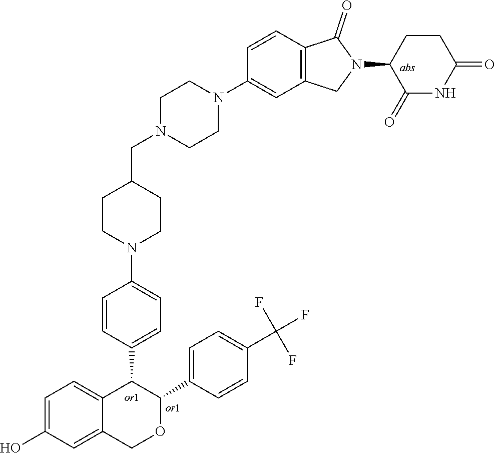 [SMILES: O=C1CC[C@H](N2Cc3cc(N4CCN(CC5CCN(c6ccc([C@H]7c8ccc(O)cc8CO[C@H]7c7ccc(C(F)(F)F)cc7)cc6)CC5)CC4)ccc3C2=O)C(=O)N1] |
| I-57 | 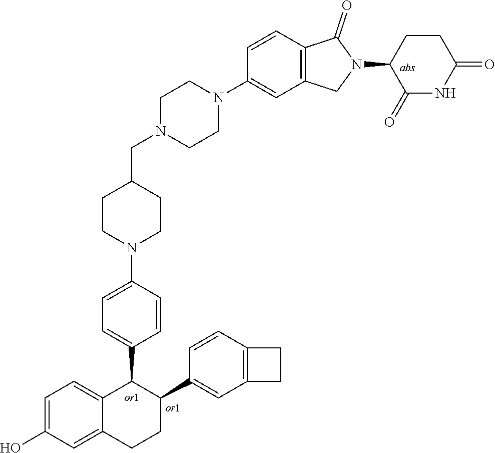 [SMILES: O=C1CC[C@H](N2Cc3cc(N4CCN(CC5CCN(c6ccc([C@@H]7c8ccc(O)cc8CC[C@@H]7c7ccc8c(c7)CC8)cc6)CC5)CC4)ccc3C2=O)C(=O)N1] |
| I-58 | 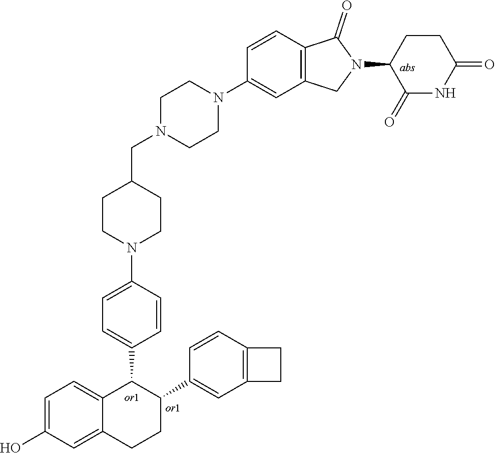 [SMILES: O=C1CC[C@H](N2Cc3cc(N4CCN(CC5CCN(c6ccc([C@H]7c8ccc(O)cc8CC[C@H]7c7ccc8c(c7)CC8)cc6)CC5)CC4)ccc3C2=O)C(=O)N1] |
| I-59 | 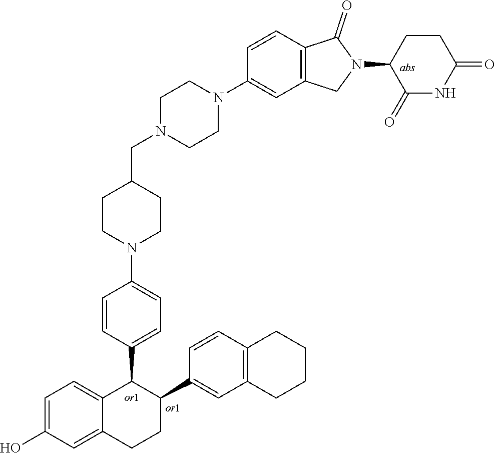 [SMILES: O=C1CC[C@H](N2Cc3cc(N4CCN(CC5CCN(c6ccc([C@@H]7c8ccc(O)cc8CC[C@@H]7c7ccc8c(c7)CCCC8)cc6)CC5)CC4)ccc3C2=O)C(=O)N1] |
| I-60 | 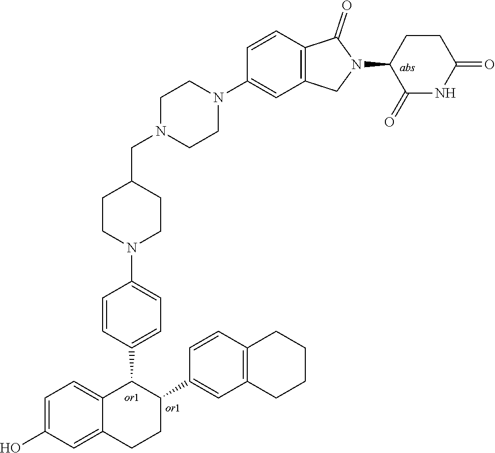 [SMILES: O=C1CC[C@H](N2Cc3cc(N4CCN(CC5CCN(c6ccc([C@H]7c8ccc(O)cc8CC[C@H]7c7ccc8c(c7)CCCC8)cc6)CC5)CC4)ccc3C2=O)C(=O)N1] |
| I-61 | 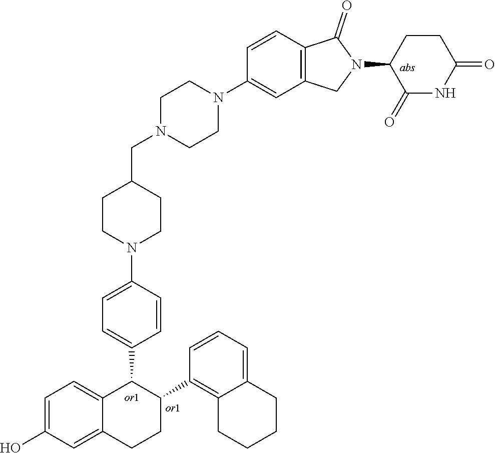 [SMILES: O=C1CC[C@H](N2Cc3cc(N4CCN(CC5CCN(c6ccc([C@H]7c8ccc(O)cc8CC[C@H]7c7cccc8c7CCCC8)cc6)CC5)CC4)ccc3C2=O)C(=O)N1] |
| I-62 | 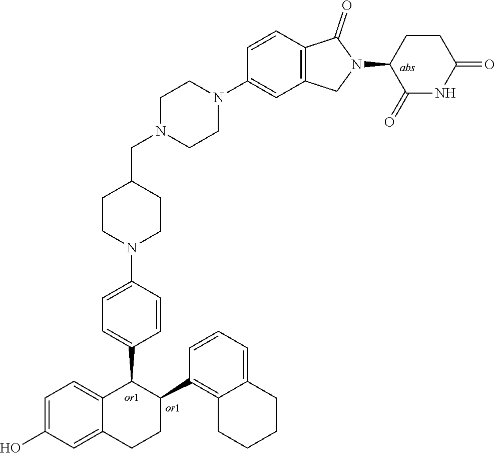 [SMILES: O=C1CC[C@H](N2Cc3cc(N4CCN(CC5CCN(c6ccc([C@@H]7c8ccc(O)cc8CC[C@@H]7c7cccc8c7CCCC8)cc6)CC5)CC4)ccc3C2=O)C(=O)N1] |
| I-63(*) | 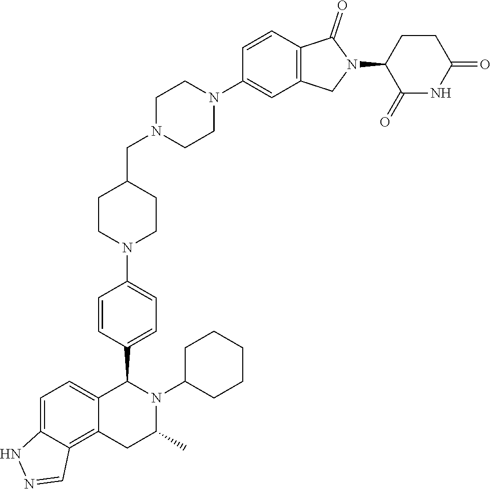 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1C1CCCCC1] |
| I-64(*) | 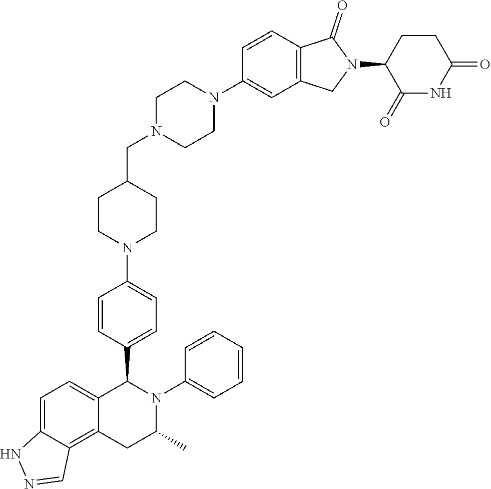 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1c1ccccc1] |
| I-65(*) | 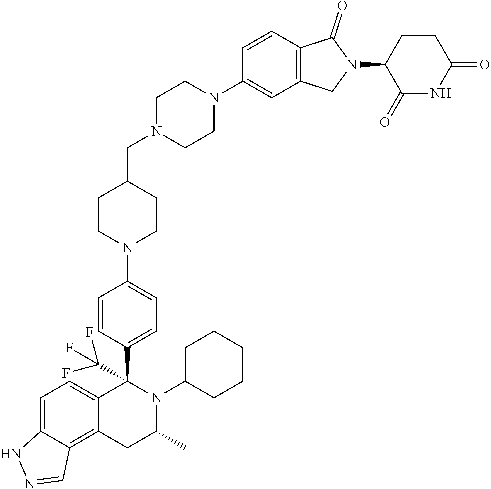 [SMILES: C[C@@H]1Cc2c(ccc3[nH]ncc23)[C@](c2ccc(N3CCC(CN4CCN(c5ccc6c(c5)CN([C@H]5CCC(=O)NC5=O)C6=O)CC4)CC3)cc2)(C(F)(F)F)N1C1CCCCC1] |
| I-66(*) | 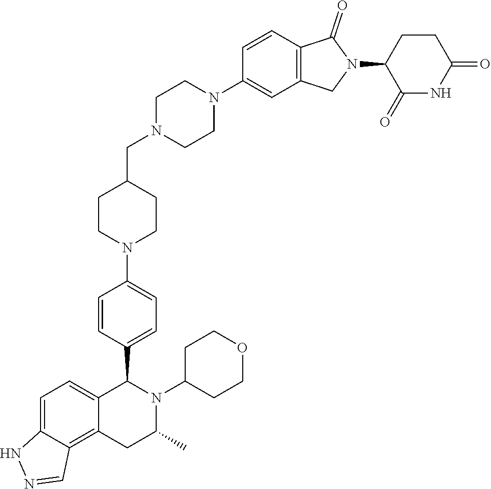 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1C1CCOCC1] |
| I-67(*) | 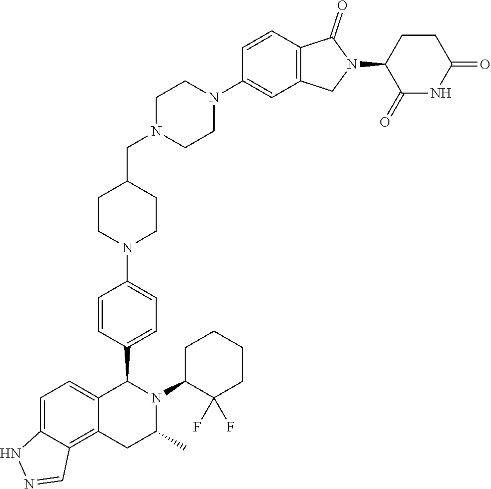 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1[C@H]1CCCCC1(F)F] |
| I-68(*) | 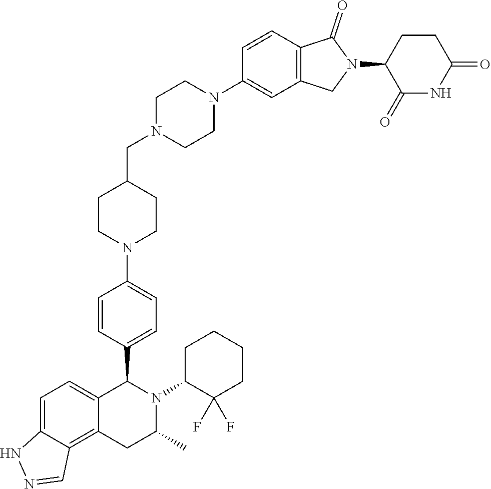 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1[C@@H]1CCCCC1(F)F] |
| I-69(*) | 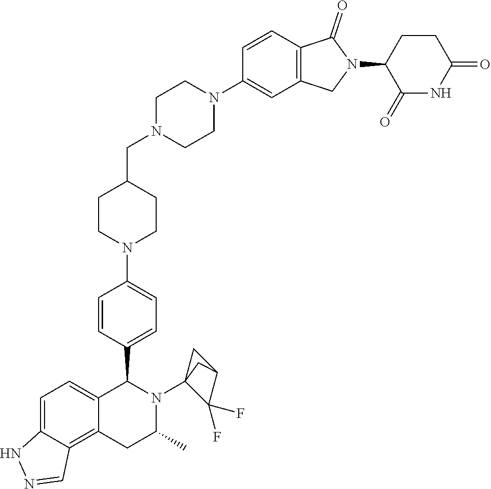 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1C12CC(C1)C2(F)F] |
| I-70(*) | 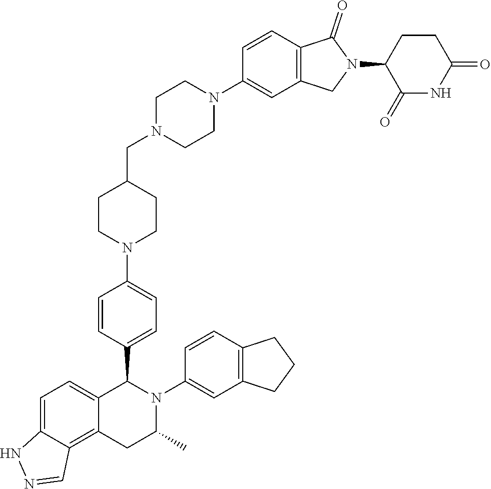 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1c1ccc2c(c1)CCC2] |
| I-71(*) | 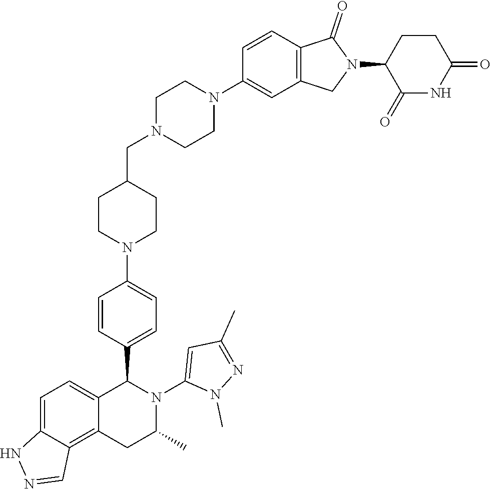 [SMILES: Cc1cc(N2[C@H](c3ccc(N4CCC(CN5CCN(c6ccc7c(c6)CN([C@H]6CCC(=O)NC6=O)C7=O)CC5)CC4)cc3)c3ccc4[nH]ncc4c3C[C@H]2C)n(C)n1] |
| I-72(*) | 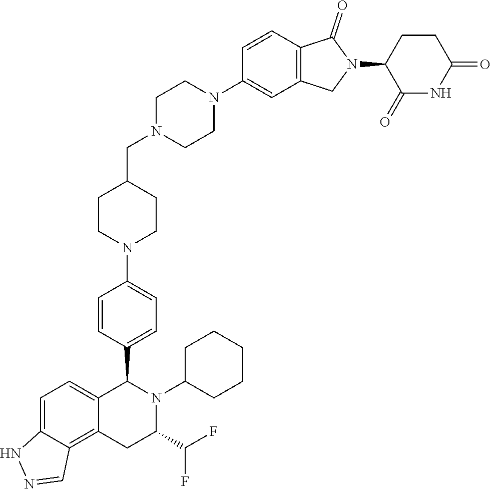 [SMILES: O=C1CC[C@H](N2Cc3cc(N4CCN(CC5CCN(c6ccc([C@@H]7c8ccc9[nH]ncc9c8C[C@@H](C(F)F)N7C7CCCCC7)cc6)CC5)CC4)ccc3C2=O)C(=O)N1] |
| I-73(*) | 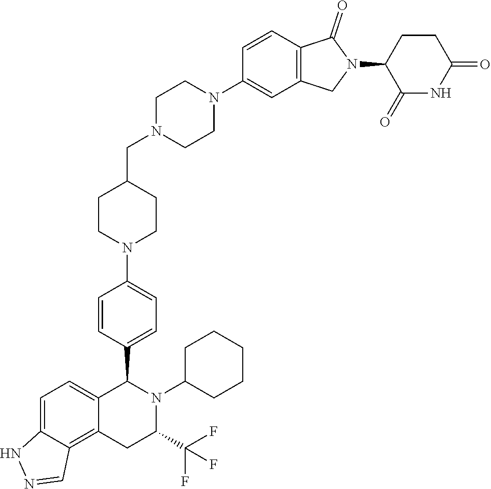 [SMILES: O=C1CC[C@H](N2Cc3cc(N4CCN(CC5CCN(c6ccc([C@@H]7c8ccc9[nH]ncc9c8C[C@@H](C(F)(F)F)N7C7CCCCC7)cc6)CC5)CC4)ccc3C2=O)C(=O)N1] |
| I-74(*) | 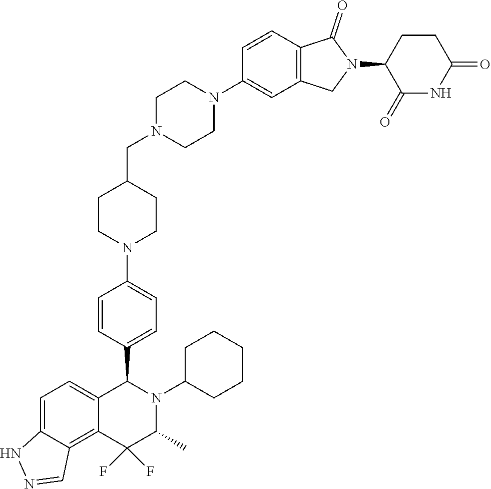 [SMILES: C[C@H]1N(C2CCCCC2)[C@H](c2ccc(N3CCC(CN4CCN(c5ccc6c(c5)CN([C@H]5CCC(=O)NC5=O)C6=O)CC4)CC3)cc2)c2ccc3[nH]ncc3c2C1(F)F] |
| I-76 | 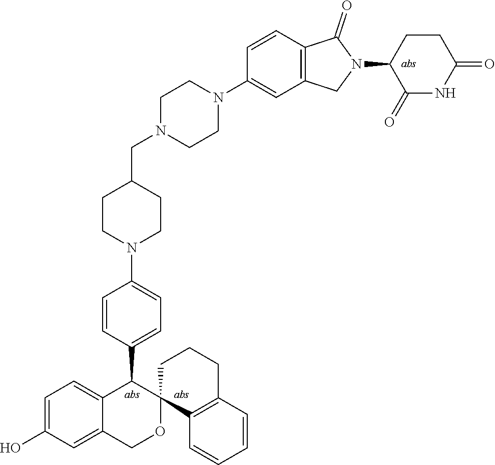 [SMILES: O=C1CC[C@H](N2Cc3cc(N4CCN(CC5CCN(c6ccc([C@@H]7c8ccc(O)cc8CO[C@@]78CCCc7ccccc78)cc6)CC5)CC4)ccc3C2=O)C(=O)N1] |
| I-77 | 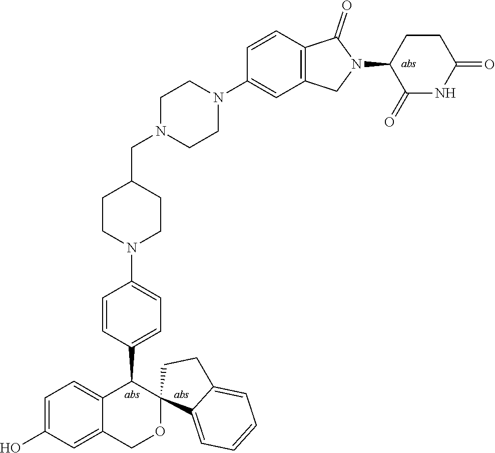 [SMILES: O=C1CC[C@H](N2Cc3cc(N4CCN(CC5CCN(c6ccc([C@@H]7c8ccc(O)cc8CO[C@@]78CCc7ccccc78)cc6)CC5)CC4)ccc3C2=O)C(=O)N1] |
| I-78(*) | 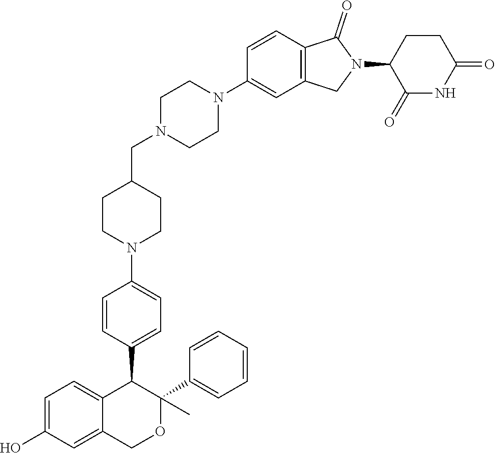 [SMILES: C[C@@]1(c2ccccc2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-79(*) | 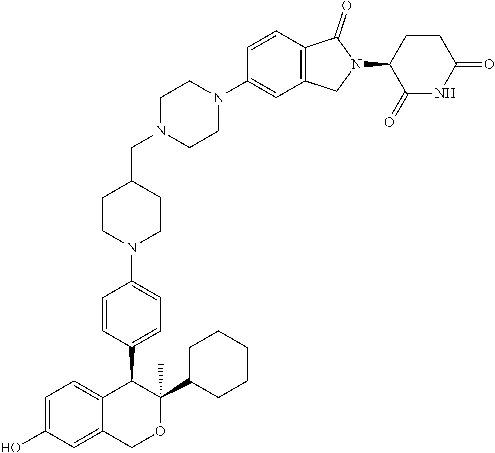 [SMILES: C[C@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-80(*) | 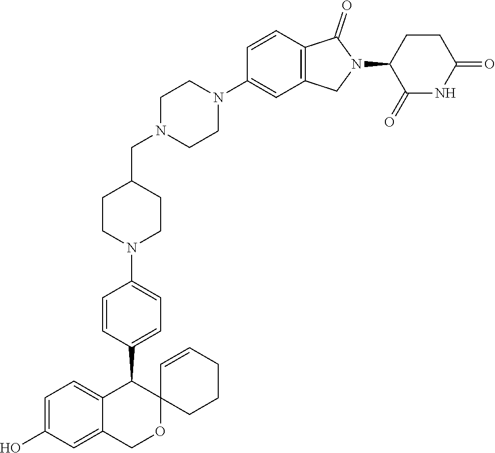 [SMILES: O=C1CC[C@H](N2Cc3cc(N4CCN(CC5CCN(c6ccc([C@@H]7c8ccc(O)cc8COC78C=CCCC8)cc6)CC5)CC4)ccc3C2=O)C(=O)N1] |
| I-81(*) | 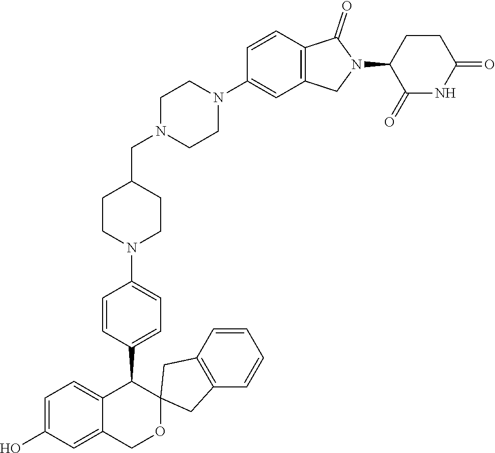 [SMILES: O=C1CC[C@H](N2Cc3cc(N4CCN(CC5CCN(c6ccc([C@@H]7c8ccc(O)cc8COC78Cc7ccccc7C8)cc6)CC5)CC4)ccc3C2=O)C(=O)N1] |
| I-83 | 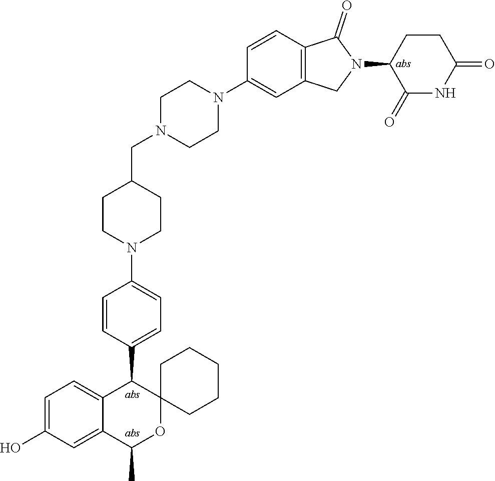 [SMILES: C[C@@H]1OC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-84(*) | 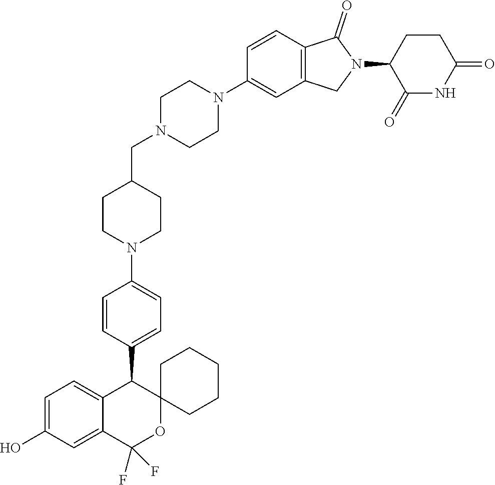 [SMILES: O=C1CC[C@H](N2Cc3cc(N4CCN(CC5CCN(c6ccc([C@@H]7c8ccc(O)cc8C(F)(F)OC78CCCCC8)cc6)CC5)CC4)ccc3C2=O)C(=O)N1] |
| I-85 | 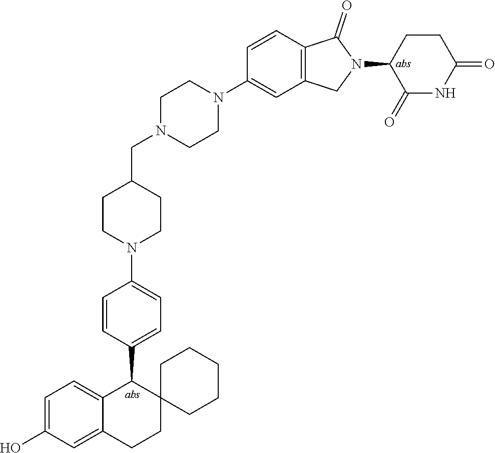 [SMILES: O=C1CC[C@H](N2Cc3cc(N4CCN(CC5CCN(c6ccc([C@@H]7c8ccc(O)cc8CCC78CCCCC8)cc6)CC5)CC4)ccc3C2=O)C(=O)N1] |
| I-87(*) | 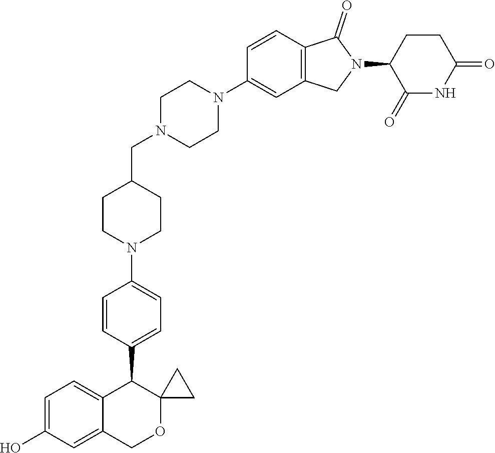 [SMILES: O=C1CC[C@H](N2Cc3cc(N4CCN(CC5CCN(c6ccc([C@@H]7c8ccc(O)cc8COC78CC8)cc6)CC5)CC4)ccc3C2=O)C(=O)N1] |
| I-88(*) | 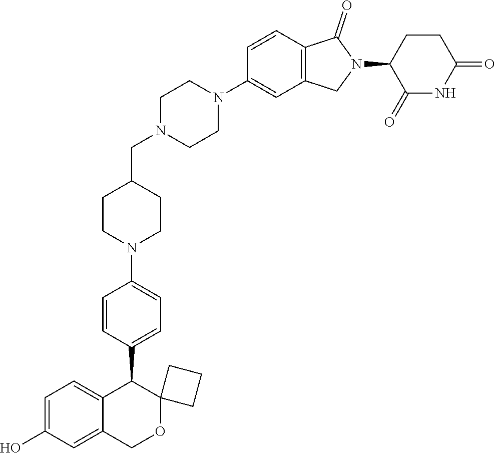 [SMILES: O=C1CC[C@H](N2Cc3cc(N4CCN(CC5CCN(c6ccc([C@@H]7c8ccc(O)cc8COC78CCC8)cc6)CC5)CC4)ccc3C2=O)C(=O)N1] |
| I-89(*) | 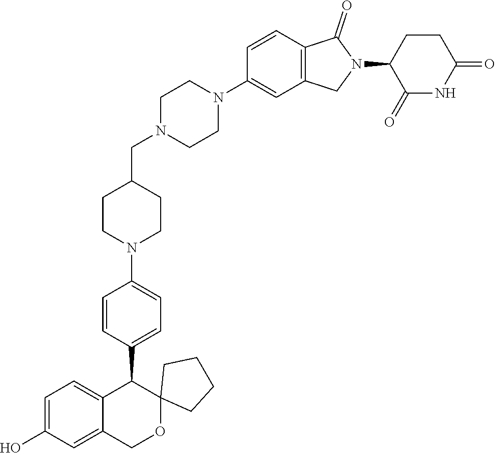 [SMILES: O=C1CC[C@H](N2Cc3cc(N4CCN(CC5CCN(c6ccc([C@@H]7c8ccc(O)cc8COC78CCCC8)cc6)CC5)CC4)ccc3C2=O)C(=O)N1] |
| I-91(*) | 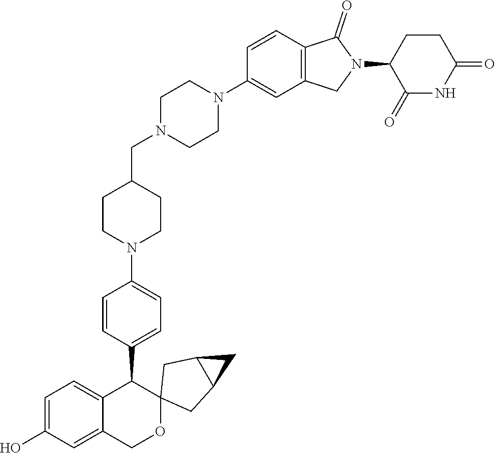 [SMILES: O=C1CC[C@H](N2Cc3cc(N4CCN(CC5CCN(c6ccc([C@@H]7c8ccc(O)cc8COC78C[C@H]7C[C@H]7C8)cc6)CC5)CC4)ccc3C2=O)C(=O)N1] |
| I-92(*) | 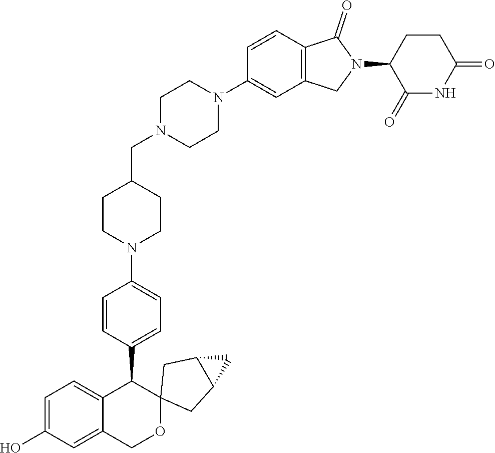 [SMILES: O=C1CC[C@H](N2Cc3cc(N4CCN(CC5CCN(c6ccc([C@@H]7c8ccc(O)cc8COC78C[C@H]7C[C@H]7C8)cc6)CC5)CC4)ccc3C2=O)C(=O)N1] |
| I-93(*) | 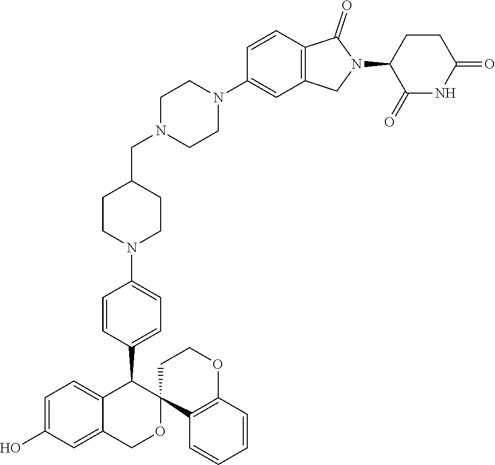 [SMILES: O=C1CC[C@H](N2Cc3cc(N4CCN(CC5CCN(c6ccc([C@@H]7c8ccc(O)cc8CO[C@@]78CCOc7ccccc78)cc6)CC5)CC4)ccc3C2=O)C(=O)N1] |
| I-94(*) | 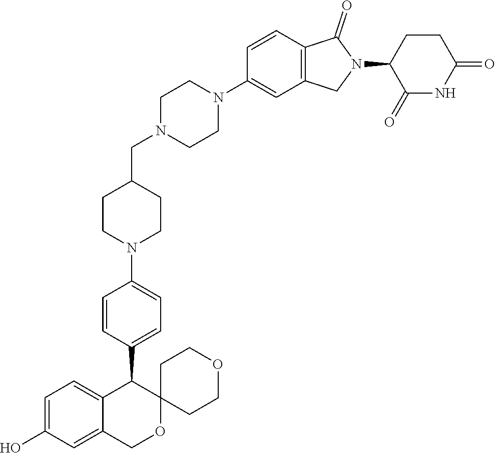 [SMILES: O=C1CC[C@H](N2Cc3cc(N4CCN(CC5CCN(c6ccc([C@@H]7c8ccc(O)cc8COC78CCOCC8)cc6)CC5)CC4)ccc3C2=O)C(=O)N1] |
| I-95(*) | 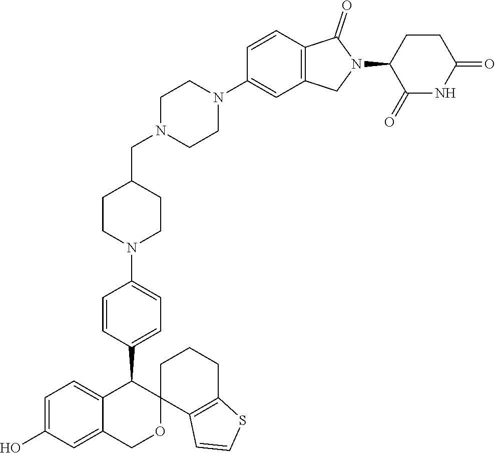 [SMILES: O=C1CC[C@H](N2Cc3cc(N4CCN(CC5CCN(c6ccc([C@@H]7c8ccc(O)cc8COC78CCCc7sccc78)cc6)CC5)CC4)ccc3C2=O)C(=O)N1] |
| I-96(*) | 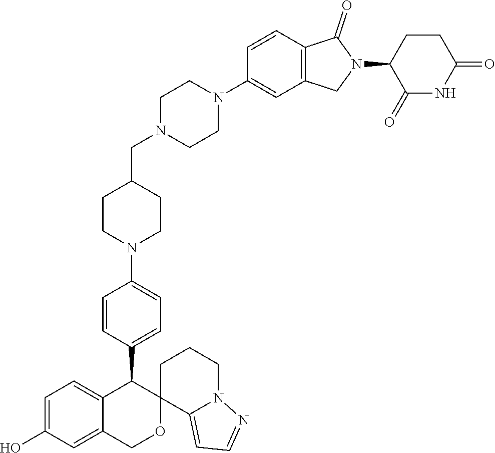 [SMILES: O=C1CC[C@H](N2Cc3cc(N4CCN(CC5CCN(c6ccc([C@@H]7c8ccc(O)cc8COC78CCCn7nccc78)cc6)CC5)CC4)ccc3C2=O)C(=O)N1] |
| I-97(*) | 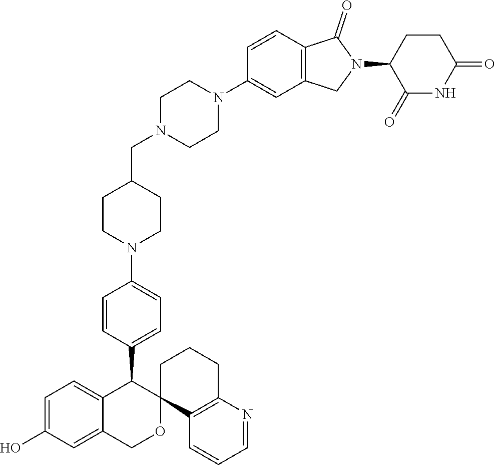 [SMILES: O=C1CC[C@H](N2Cc3cc(N4CCN(CC5CCN(c6ccc([C@@H]7c8ccc(O)cc8CO[C@@]78CCCc7ncccc78)cc6)CC5)CC4)ccc3C2=O)C(=O)N1] |
| I-98‡ | 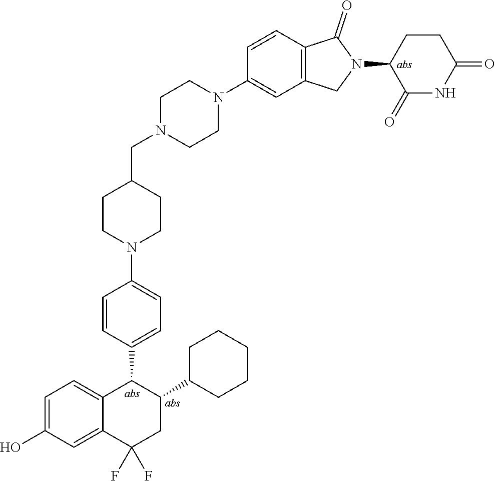 [SMILES: O=C1CC[C@H](N2Cc3cc(N4CCN(CC5CCN(c6ccc([C@H]7c8ccc(O)cc8C(F)(F)C[C@H]7C7CCCCC7)cc6)CC5)CC4)ccc3C2=O)C(=O)N1] |
| I-99‡ | 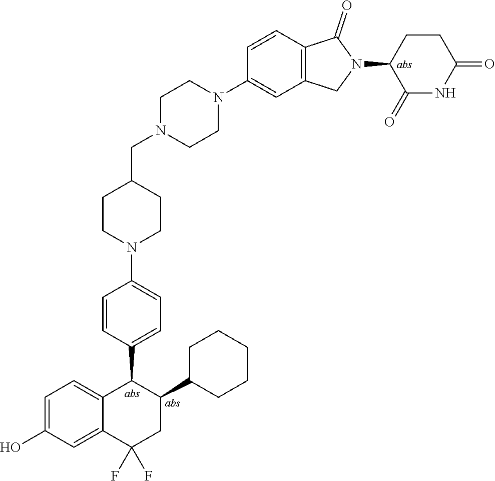 [SMILES: O=C1CC[C@H](N2Cc3cc(N4CCN(CC5CCN(c6ccc([C@@H]7c8ccc(O)cc8C(F)(F)C[C@@H]7C7CCCCC7)cc6)CC5)CC4)ccc3C2=O)C(=O)N1] |
| I-100 | 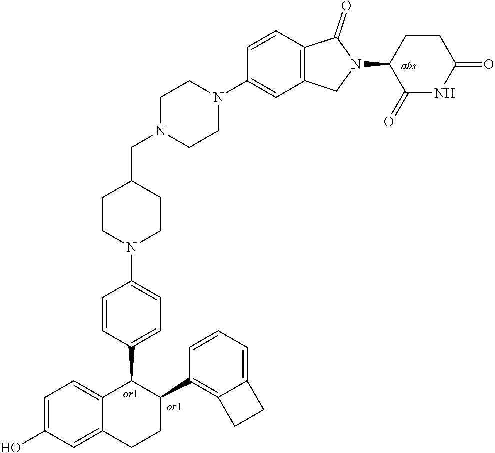 [SMILES: O=C1CC[C@H](N2Cc3cc(N4CCN(CC5CCN(c6ccc([C@@H]7c8ccc(O)cc8CC[C@@H]7c7cccc8c7CC8)cc6)CC5)CC4)ccc3C2=O)C(=O)N1] |
| I-101 | 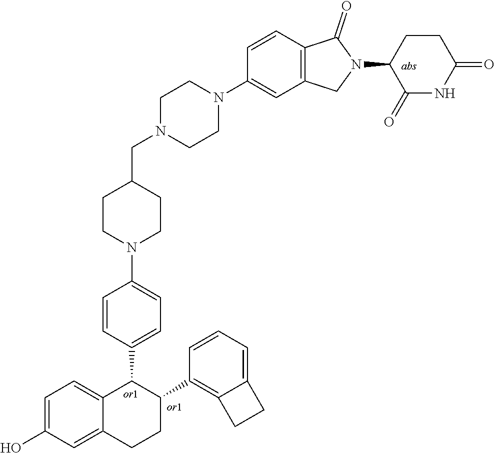 [SMILES: O=C1CC[C@H](N2Cc3cc(N4CCN(CC5CCN(c6ccc([C@H]7c8ccc(O)cc8CC[C@H]7c7cccc8c7CC8)cc6)CC5)CC4)ccc3C2=O)C(=O)N1] |
| I-102 | 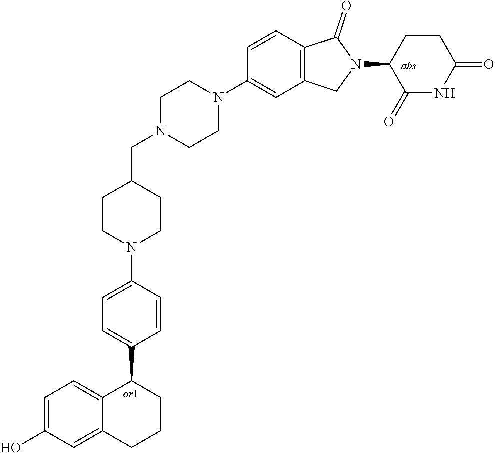 [SMILES: O=C1CC[C@H](N2Cc3cc(N4CCN(CC5CCN(c6ccc([C@H]7CCCc8cc(O)ccc87)cc6)CC5)CC4)ccc3C2=O)C(=O)N1] |
| I-103 | 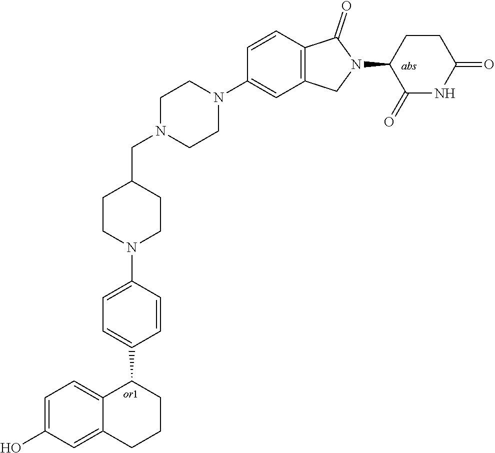 [SMILES: O=C1CC[C@H](N2Cc3cc(N4CCN(CC5CCN(c6ccc([C@@H]7CCCc8cc(O)ccc87)cc6)CC5)CC4)ccc3C2=O)C(=O)N1] |
| I-104 | 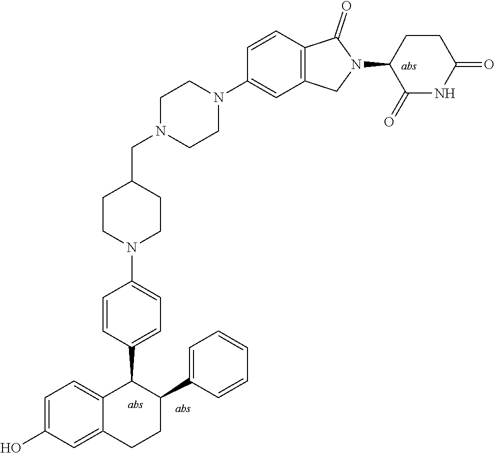 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] |
| I-105 | 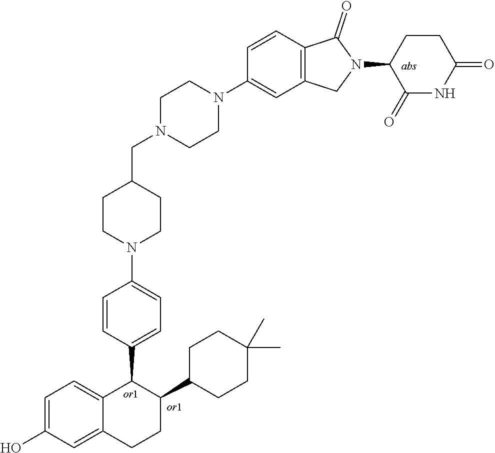 [SMILES: CC1(C)CCC([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)CC1] |
| I-106 | 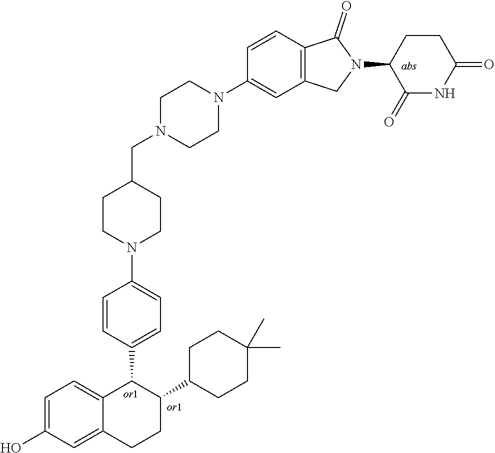 [SMILES: CC1(C)CCC([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)CC1] |
| I-107 | 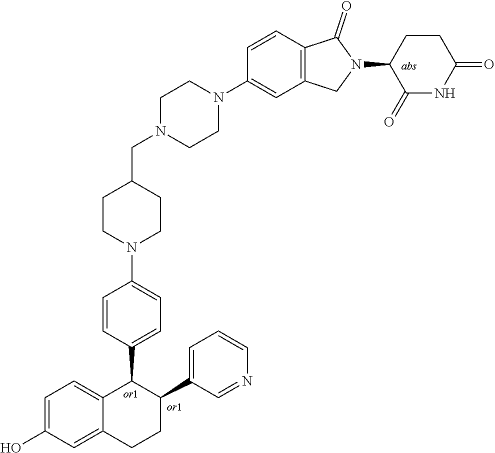 [SMILES: O=C1CC[C@H](N2Cc3cc(N4CCN(CC5CCN(c6ccc([C@@H]7c8ccc(O)cc8CC[C@@H]7c7cccnc7)cc6)CC5)CC4)ccc3C2=O)C(=O)N1] |
| I-108 | 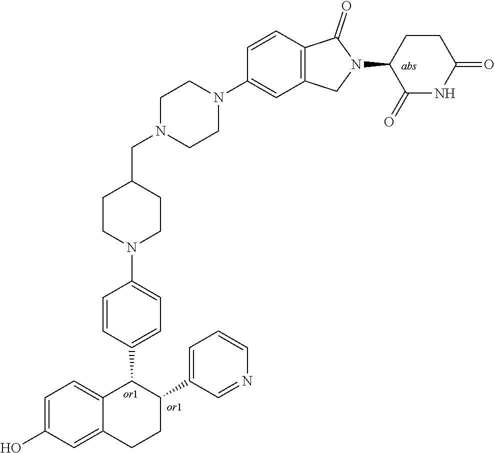 [SMILES: O=C1CC[C@H](N2Cc3cc(N4CCN(CC5CCN(c6ccc([C@H]7c8ccc(O)cc8CC[C@H]7c7cccnc7)cc6)CC5)CC4)ccc3C2=O)C(=O)N1] |
| I-109 | 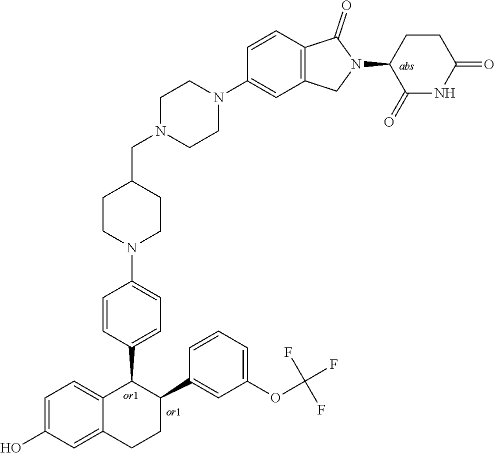 [SMILES: O=C1CC[C@H](N2Cc3cc(N4CCN(CC5CCN(c6ccc([C@@H]7c8ccc(O)cc8CC[C@@H]7c7cccc(OC(F)(F)F)c7)cc6)CC5)CC4)ccc3C2=O)C(=O)N1] |
| I-110 | 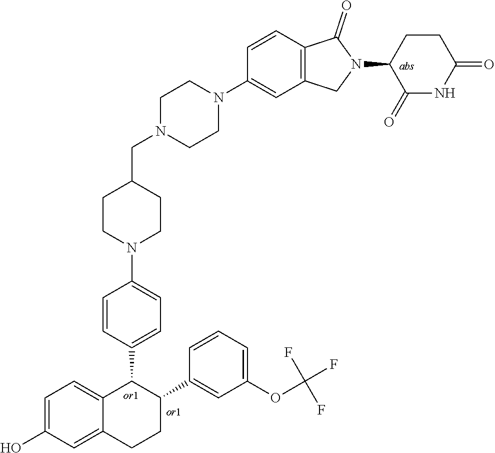 [SMILES: O=C1CC[C@H](N2Cc3cc(N4CCN(CC5CCN(c6ccc([C@H]7c8ccc(O)cc8CC[C@H]7c7cccc(OC(F)(F)F)c7)cc6)CC5)CC4)ccc3C2=O)C(=O)N1] |
| I-111 | 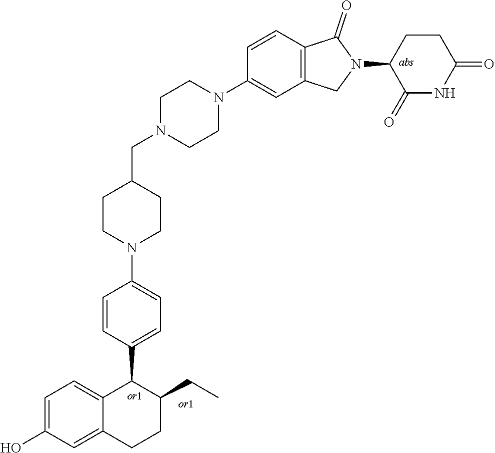 [SMILES: CC[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-112 | 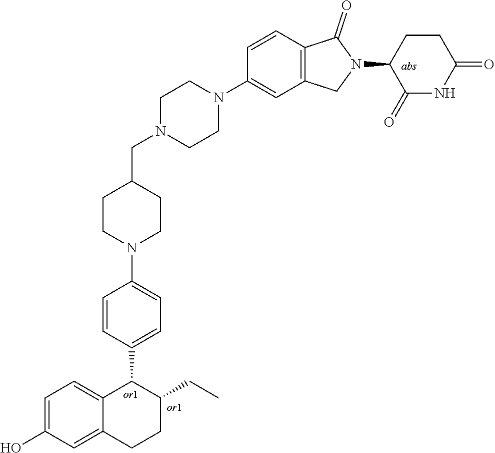 [SMILES: CC[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-114‡ | 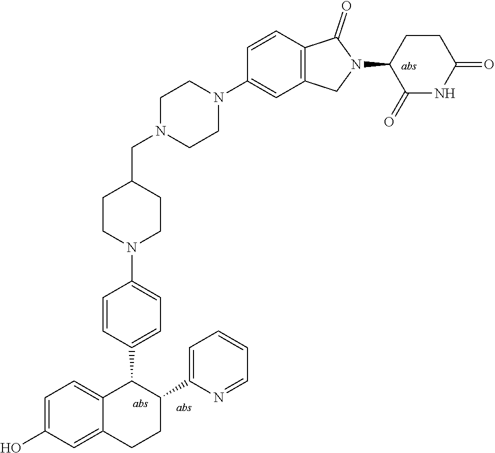 [SMILES: O=C1CC[C@H](N2Cc3cc(N4CCN(CC5CCN(c6ccc([C@H]7c8ccc(O)cc8CC[C@H]7c7ccccn7)cc6)CC5)CC4)ccc3C2=O)C(=O)N1] |
| I-115‡ | 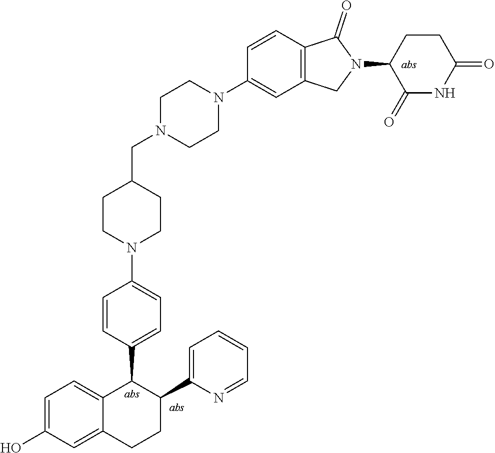 [SMILES: O=C1CC[C@H](N2Cc3cc(N4CCN(CC5CCN(c6ccc([C@@H]7c8ccc(O)cc8CC[C@@H]7c7ccccn7)cc6)CC5)CC4)ccc3C2=O)C(=O)N1] |
| I-116‡ | 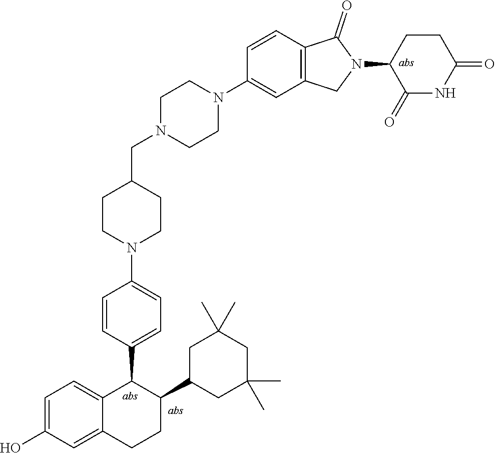 [SMILES: CC1(C)CC([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)CC(C)(C)C1] |
| I-117‡ | 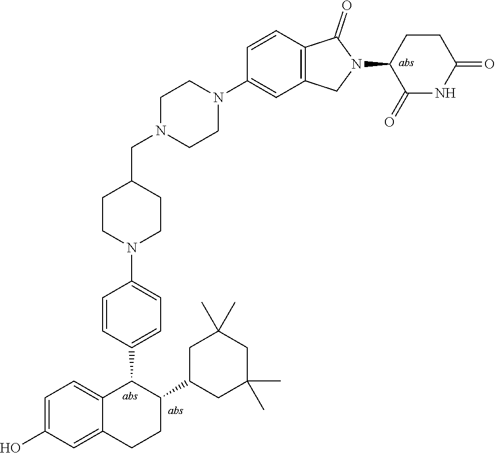 [SMILES: CC1(C)CC([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)CC(C)(C)C1] |
| I-118‡ | 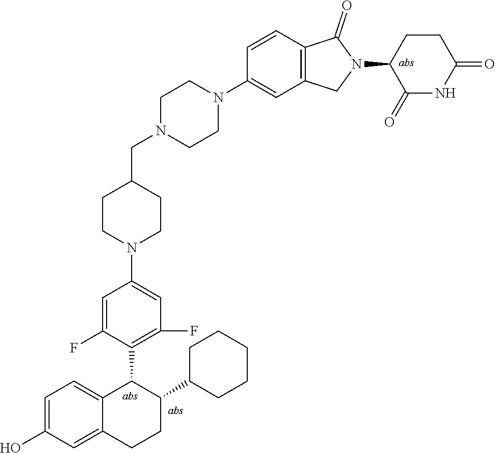 [SMILES: O=C1CC[C@H](N2Cc3cc(N4CCN(CC5CCN(c6cc(F)c([C@H]7c8ccc(O)cc8CC[C@H]7C7CCCCC7)c(F)c6)CC5)CC4)ccc3C2=O)C(=O)N1] |
| I-119‡ | 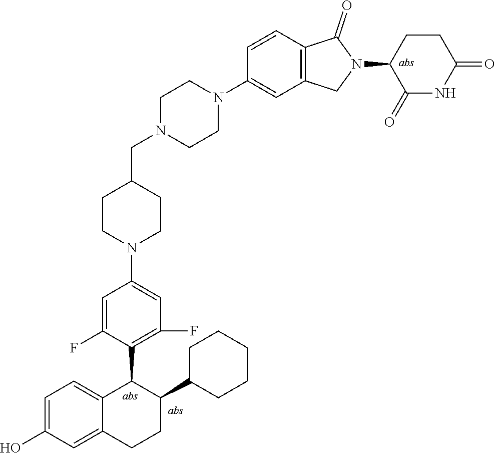 [SMILES: O=C1CC[C@H](N2Cc3cc(N4CCN(CC5CCN(c6cc(F)c([C@@H]7c8ccc(O)cc8CC[C@@H]7C7CCCCC7)c(F)c6)CC5)CC4)ccc3C2=O)C(=O)N1] |
| I-120‡ | 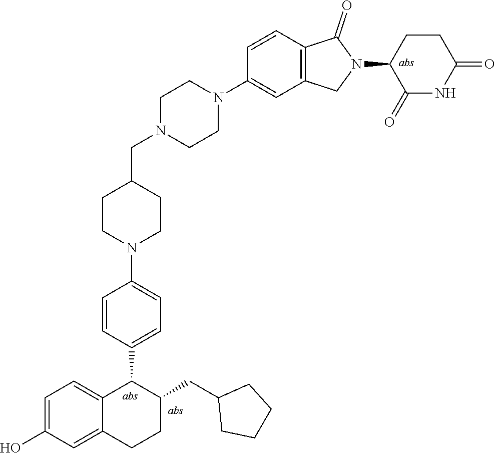 [SMILES: O=C1CC[C@H](N2Cc3cc(N4CCN(CC5CCN(c6ccc([C@H]7c8ccc(O)cc8CC[C@H]7CC7CCCC7)cc6)CC5)CC4)ccc3C2=O)C(=O)N1] |
| I-120‡ | 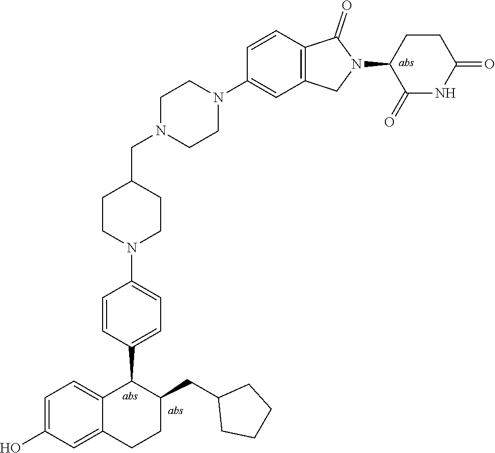 [SMILES: O=C1CC[C@H](N2Cc3cc(N4CCN(CC5CCN(c6ccc([C@@H]7c8ccc(O)cc8CC[C@@H]7CC7CCCC7)cc6)CC5)CC4)ccc3C2=O)C(=O)N1] |
| I-120‡ | 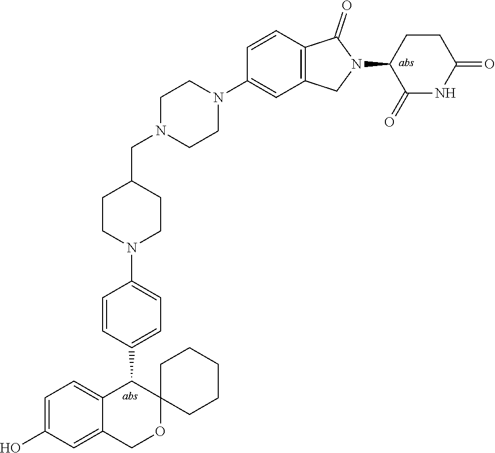 [SMILES: O=C1CC[C@H](N2Cc3cc(N4CCN(CC5CCN(c6ccc([C@H]7c8ccc(O)cc8COC78CCCCC8)cc6)CC5)CC4)ccc3C2=O)C(=O)N1] |
| I-120‡ | 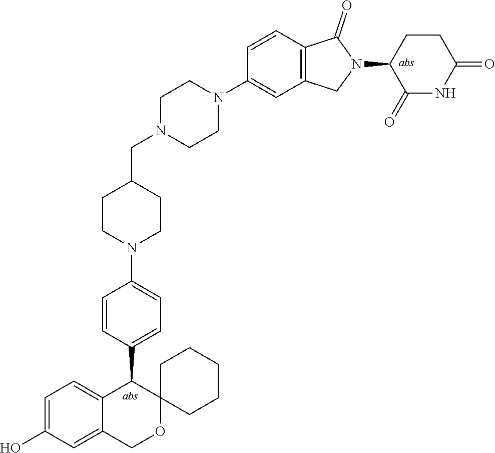 [SMILES: O=C1CC[C@H](N2Cc3cc(N4CCN(CC5CCN(c6ccc([C@@H]7c8ccc(O)cc8COC78CCCCC8)cc6)CC5)CC4)ccc3C2=O)C(=O)N1] |
| I-120‡ | 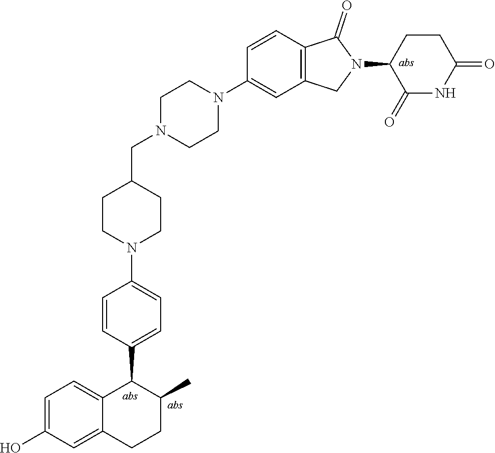 [SMILES: C[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-120‡ | 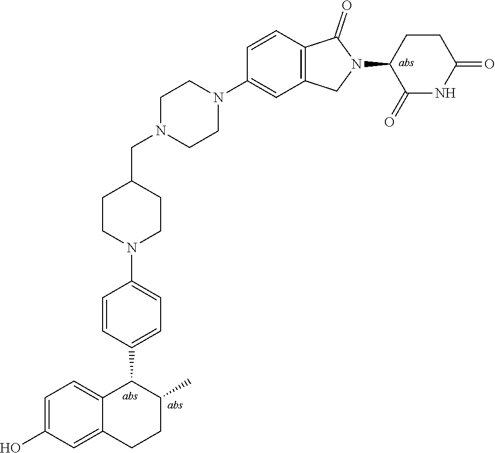 [SMILES: C[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-128 | 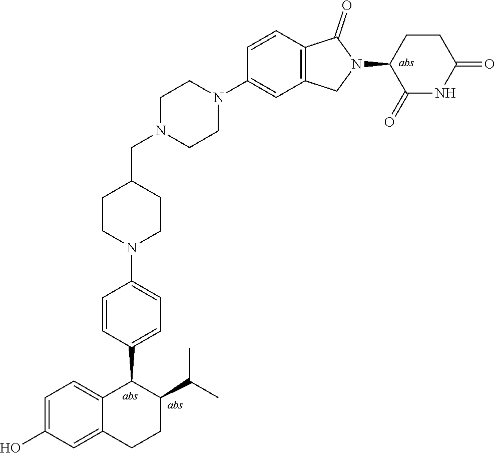 [SMILES: CC(C)[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-129 | 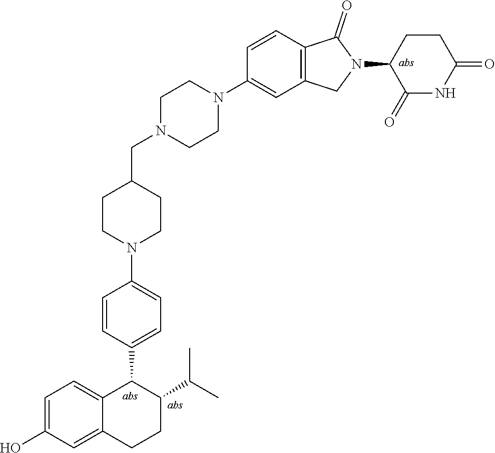 [SMILES: CC(C)[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-132 | 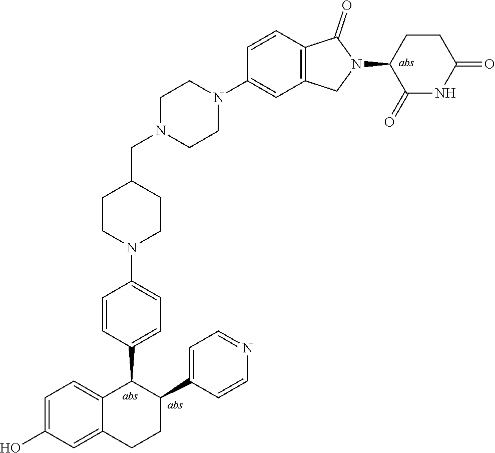 [SMILES: O=C1CC[C@H](N2Cc3cc(N4CCN(CC5CCN(c6ccc([C@@H]7c8ccc(O)cc8CC[C@@H]7c7ccncc7)cc6)CC5)CC4)ccc3C2=O)C(=O)N1] |
| I-133 | 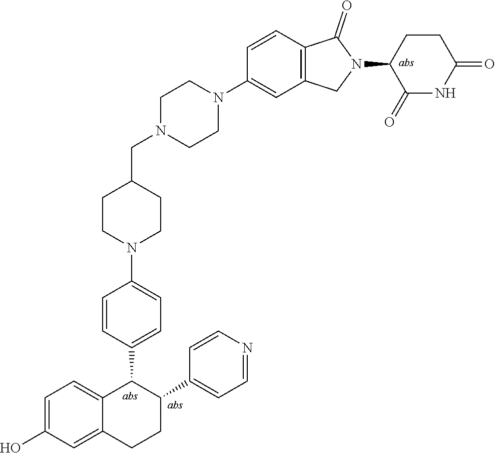 [SMILES: O=C1CC[C@H](N2Cc3cc(N4CCN(CC5CCN(c6ccc([C@H]7c8ccc(O)cc8CC[C@H]7c7ccncc7)cc6)CC5)CC4)ccc3C2=O)C(=O)N1] |
| I-134 | 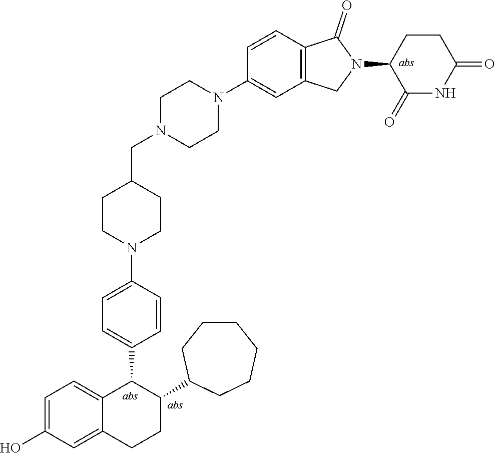 [SMILES: O=C1CC[C@H](N2Cc3cc(N4CCN(CC5CCN(c6ccc([C@H]7c8ccc(O)cc8CC[C@H]7C7CCCCCC7)cc6)CC5)CC4)ccc3C2=O)C(=O)N1] |
| I-135 | 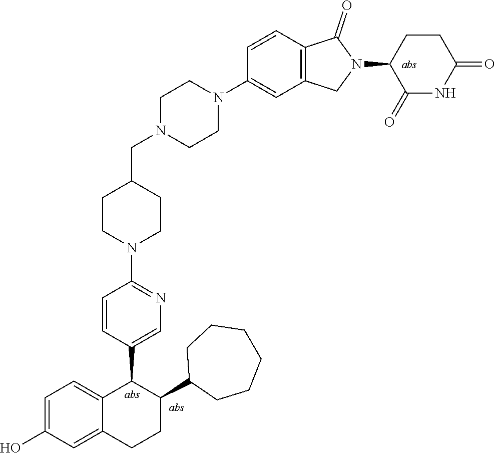 [SMILES: O=C1CC[C@H](N2Cc3cc(N4CCN(CC5CCN(c6ccc([C@@H]7c8ccc(O)cc8CC[C@@H]7C7CCCCCC7)cn6)CC5)CC4)ccc3C2=O)C(=O)N1] |
| I-136 | 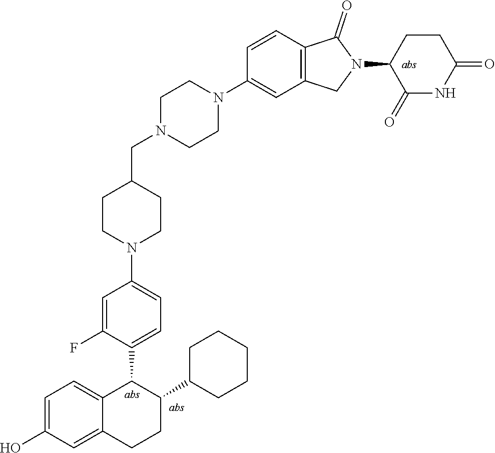 [SMILES: O=C1CC[C@H](N2Cc3cc(N4CCN(CC5CCN(c6ccc([C@H]7c8ccc(O)cc8CC[C@H]7C7CCCCC7)c(F)c6)CC5)CC4)ccc3C2=O)C(=O)N1] |
| I-137 | 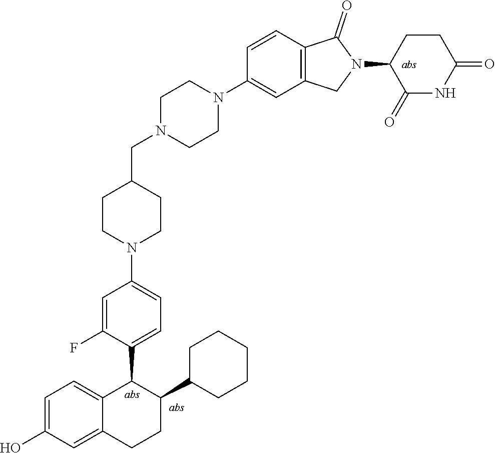 [SMILES: O=C1CC[C@H](N2Cc3cc(N4CCN(CC5CCN(c6ccc([C@@H]7c8ccc(O)cc8CC[C@@H]7C7CCCCC7)c(F)c6)CC5)CC4)ccc3C2=O)C(=O)N1] |
| I-138 | 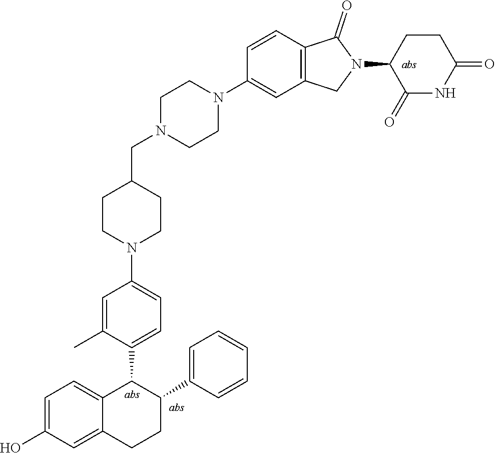 [SMILES: Cc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1c1ccccc1] |
| I-139 | 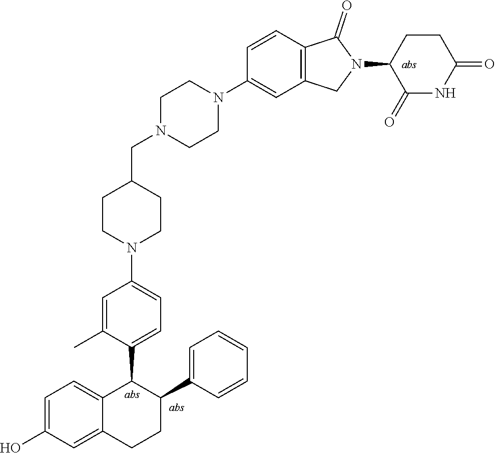 [SMILES: Cc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1c1ccccc1] |
| I-140 | 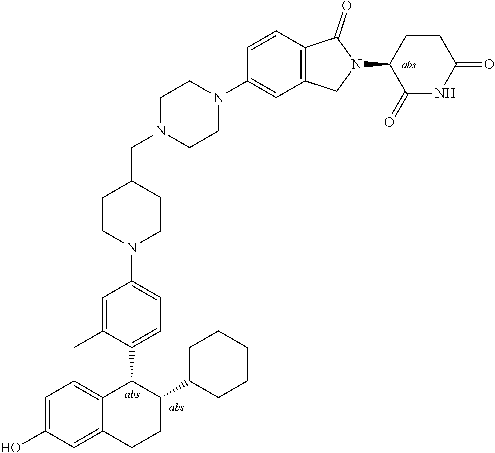 [SMILES: Cc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1C1CCCCC1] |
| I-141 | 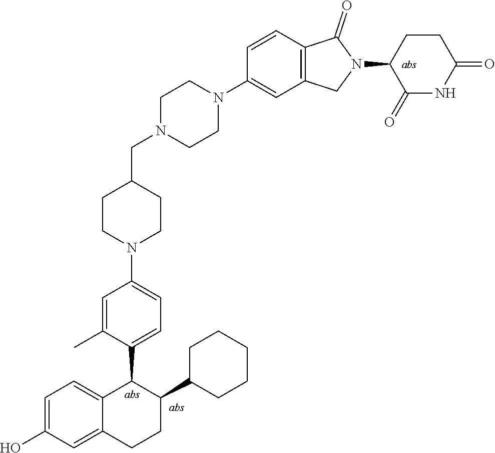 [SMILES: Cc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1C1CCCCC1] |
| I-142 | 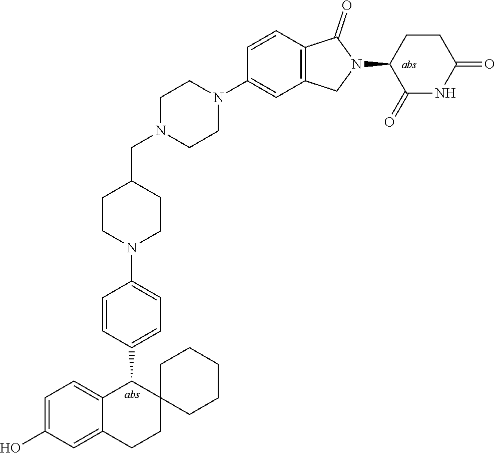 [SMILES: O=C1CC[C@H](N2Cc3cc(N4CCN(CC5CCN(c6ccc([C@H]7c8ccc(O)cc8CCC78CCCCC8)cc6)CC5)CC4)ccc3C2=O)C(=O)N1] |
| I-143 | 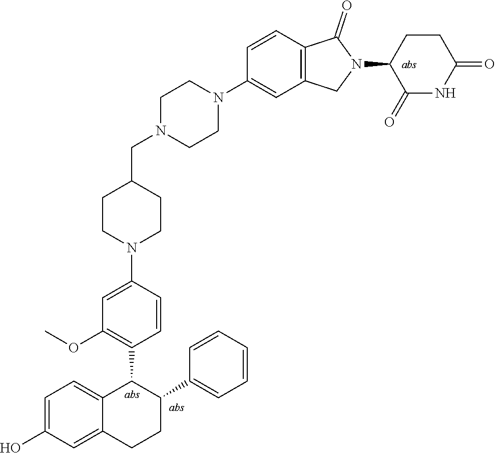 [SMILES: COc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1c1ccccc1] |
| I-144 | 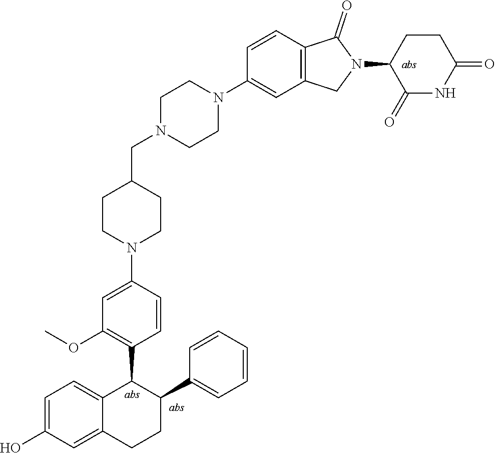 [SMILES: COc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1c1ccccc1] |
| I-145 | 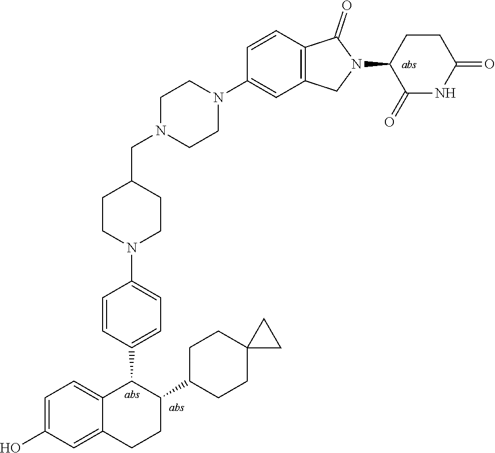 [SMILES: O=C1CC[C@H](N2Cc3cc(N4CCN(CC5CCN(c6ccc([C@H]7c8ccc(O)cc8CC[C@H]7C7CCC8(CC7)CC8)cc6)CC5)CC4)ccc3C2=O)C(=O)N1] |
| I-146 | 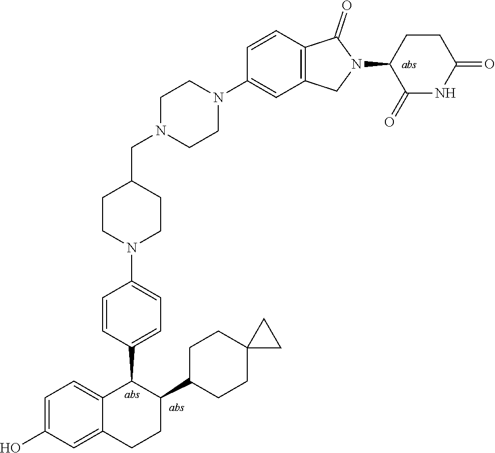 [SMILES: O=C1CC[C@H](N2Cc3cc(N4CCN(CC5CCN(c6ccc([C@@H]7c8ccc(O)cc8CC[C@@H]7C7CCC8(CC7)CC8)cc6)CC5)CC4)ccc3C2=O)C(=O)N1] |
| I-147‡ | 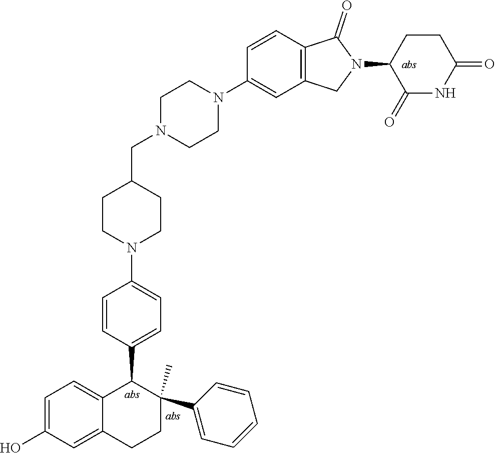 [SMILES: C[C@]1(c2ccccc2)CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-148‡ | 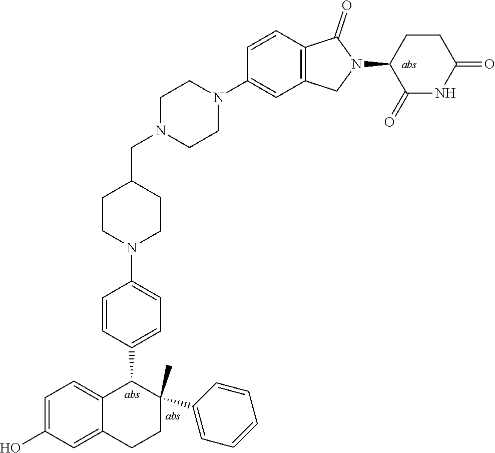 [SMILES: C[C@@]1(c2ccccc2)CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-149‡ | 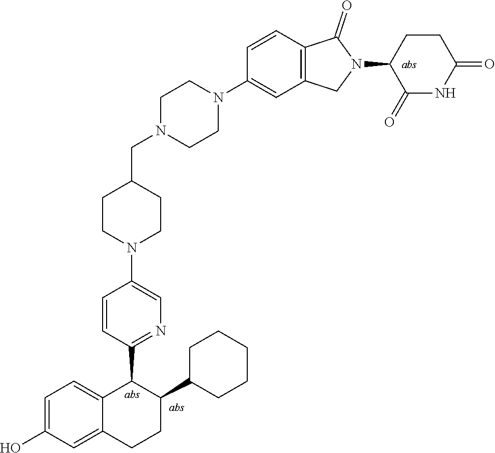 [SMILES: O=C1CC[C@H](N2Cc3cc(N4CCN(CC5CCN(c6ccc([C@@H]7c8ccc(O)cc8CC[C@@H]7C7CCCCC7)nc6)CC5)CC4)ccc3C2=O)C(=O)N1] |
| I-150‡ | 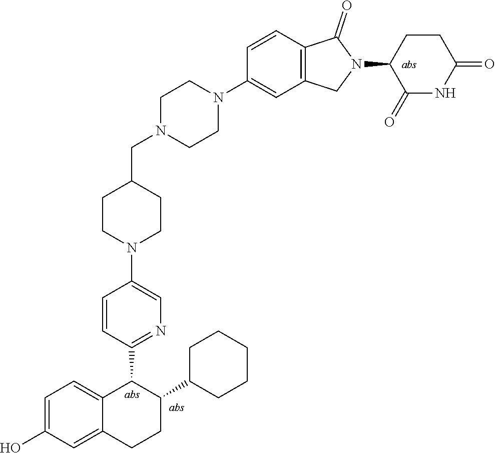 [SMILES: O=C1CC[C@H](N2Cc3cc(N4CCN(CC5CCN(c6ccc([C@H]7c8ccc(O)cc8CC[C@H]7C7CCCCC7)nc6)CC5)CC4)ccc3C2=O)C(=O)N1] |
| I-151‡ | 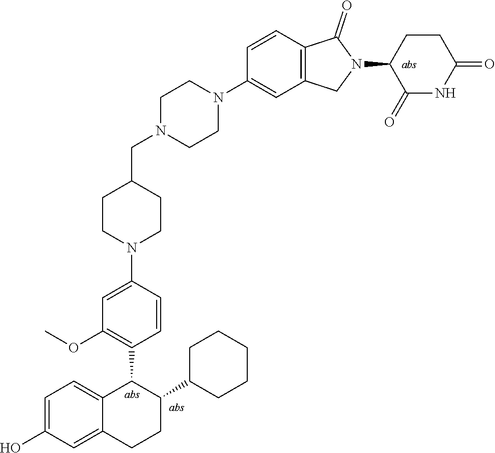 [SMILES: COc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1C1CCCCC1] |
| I-152‡ | 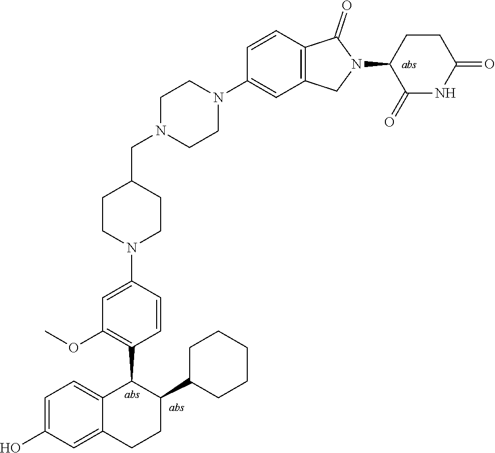 [SMILES: COc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1C1CCCCC1] |
| I-153‡ | 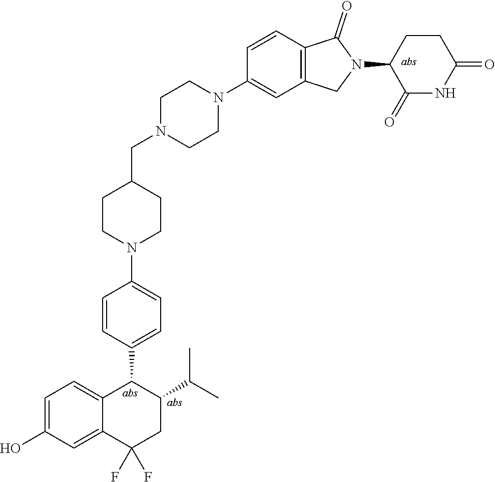 [SMILES: CC(C)[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-154‡ | 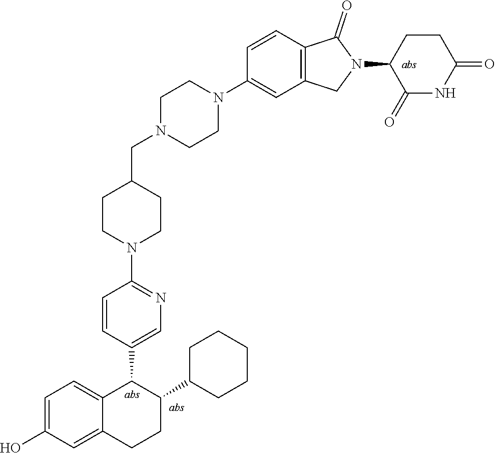 [SMILES: O=C1CC[C@H](N2Cc3cc(N4CCN(CC5CCN(c6ccc([C@H]7c8ccc(O)cc8CC[C@H]7C7CCCCC7)cn6)CC5)CC4)ccc3C2=O)C(=O)N1] |
| I-155‡ | 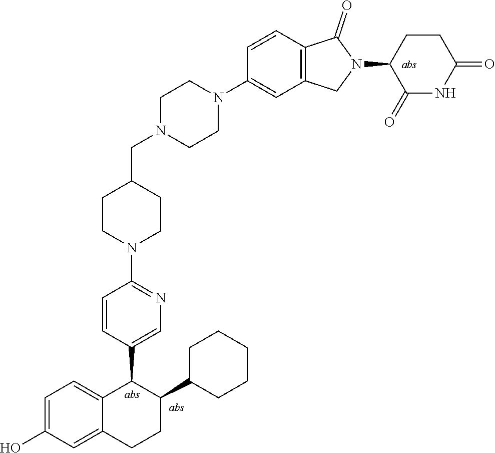 [SMILES: O=C1CC[C@H](N2Cc3cc(N4CCN(CC5CCN(c6ccc([C@@H]7c8ccc(O)cc8CC[C@@H]7C7CCCCC7)cn6)CC5)CC4)ccc3C2=O)C(=O)N1] |
| I-156‡ | 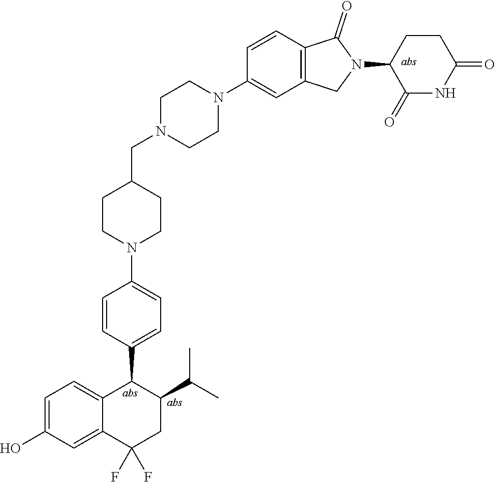 [SMILES: CC(C)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-157‡ | 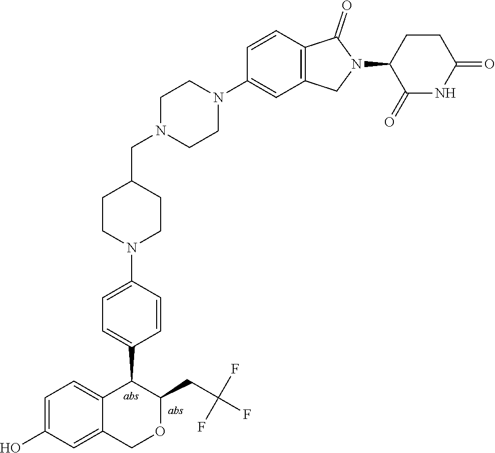 [SMILES: O=C1CC[C@H](N2Cc3cc(N4CCN(CC5CCN(c6ccc([C@@H]7c8ccc(O)cc8CO[C@@H]7CC(F)(F)F)cc6)CC5)CC4)ccc3C2=O)C(=O)N1] |
| I-158‡ | 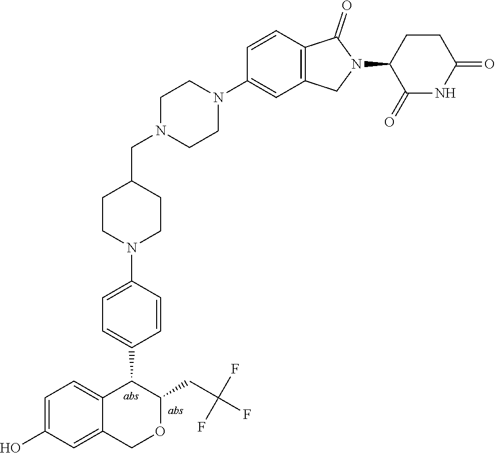 [SMILES: O=C1CC[C@H](N2Cc3cc(N4CCN(CC5CCN(c6ccc([C@H]7c8ccc(O)cc8CO[C@H]7CC(F)(F)F)cc6)CC5)CC4)ccc3C2=O)C(=O)N1] |
| I-159‡ | 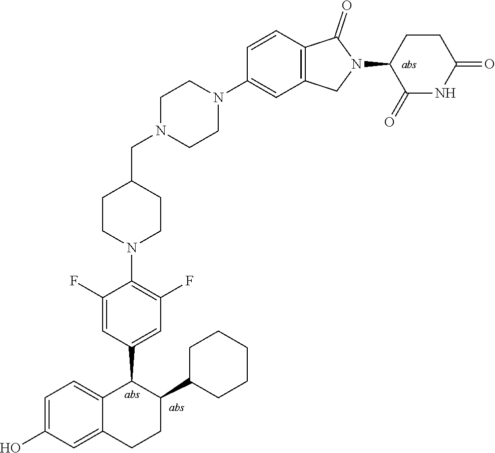 [SMILES: O=C1CC[C@H](N2Cc3cc(N4CCN(CC5CCN(c6c(F)cc([C@@H]7c8ccc(O)cc8CC[C@@H]7C7CCCCC7)cc6F)CC5)CC4)ccc3C2=O)C(=O)N1] |
| I-160‡ | 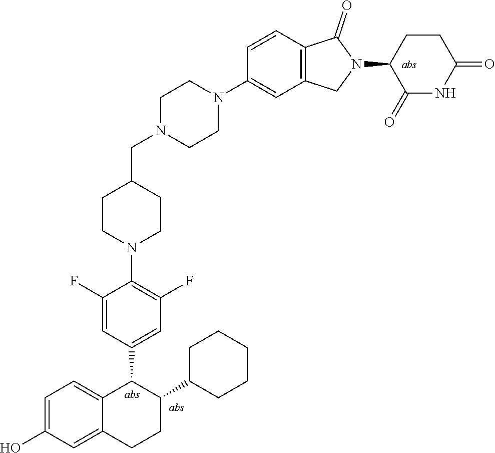 [SMILES: O=C1CC[C@H](N2Cc3cc(N4CCN(CC5CCN(c6c(F)cc([C@H]7c8ccc(O)cc8CC[C@H]7C7CCCCC7)cc6F)CC5)CC4)ccc3C2=O)C(=O)N1] |
| I-161 | 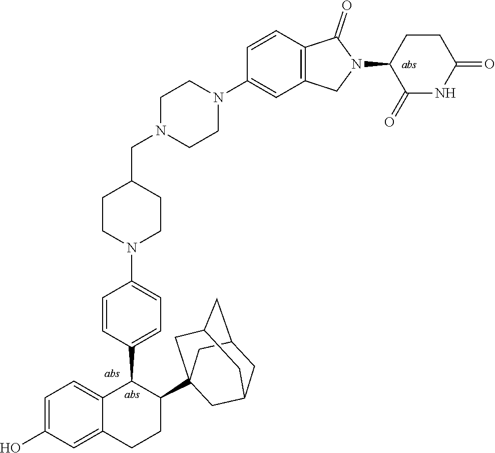 [SMILES: O=C1CC[C@H](N2Cc3cc(N4CCN(CC5CCN(c6ccc([C@@H]7c8ccc(O)cc8CC[C@@H]7C78CC9CC(CC(C9)C7)C8)cc6)CC5)CC4)ccc3C2=O)C(=O)N1] |
| I-162 | 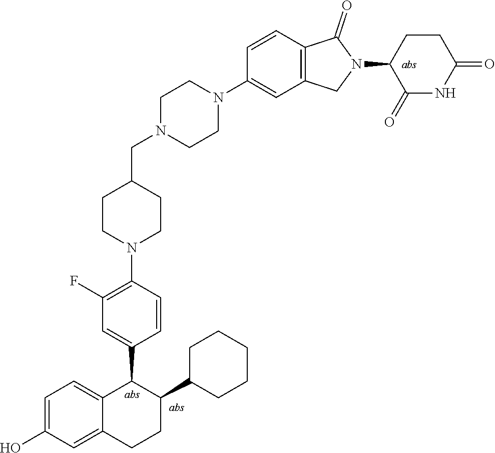 [SMILES: O=C1CC[C@H](N2Cc3cc(N4CCN(CC5CCN(c6ccc([C@@H]7c8ccc(O)cc8CC[C@@H]7C7CCCCC7)cc6F)CC5)CC4)ccc3C2=O)C(=O)N1] |
| I-163 | 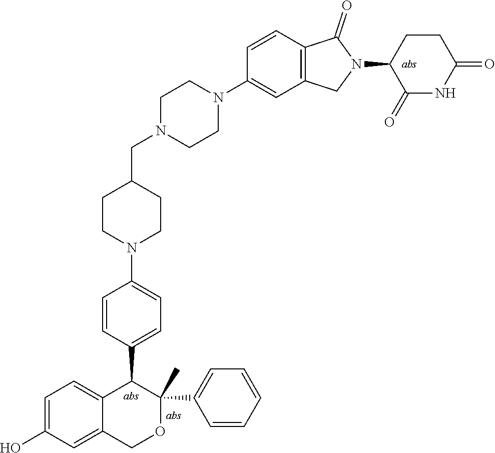 [SMILES: C[C@@]1(c2ccccc2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-164‡ | 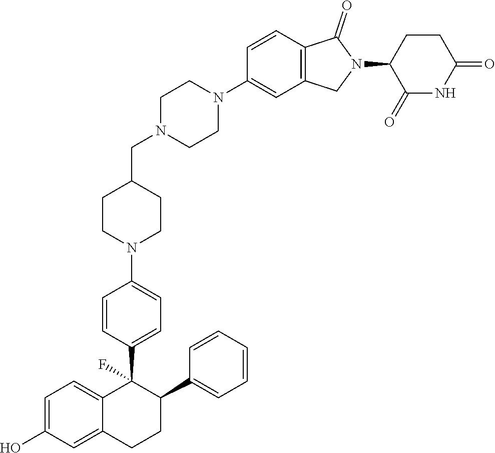 [SMILES: O=C1CC[C@H](N2Cc3cc(N4CCN(CC5CCN(c6ccc([C@]7(F)c8ccc(O)cc8CC[C@@H]7c7ccccc7)cc6)CC5)CC4)ccc3C2=O)C(=O)N1] |
| I-165‡ | 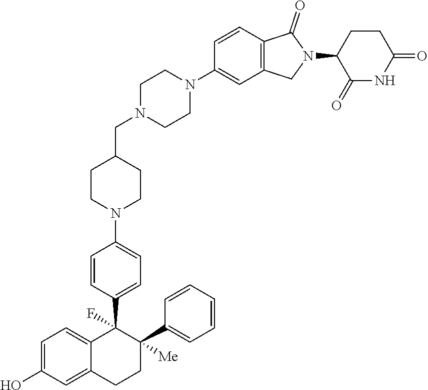 [SMILES: C[C@]1(c2ccccc2)CCc2cc(O)ccc2[C@@]1(F)c1ccc(N2CCC(CN3CCN(c4ccc5c(c4)CN([C@H]4CCC(=O)NC4=O)C5=O)CC3)CC2)cc1] |
| I-166‡ | 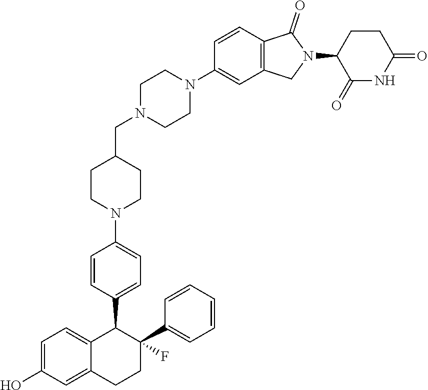 [SMILES: O=C1CC[C@H](N2Cc3cc(N4CCN(CC5CCN(c6ccc([C@@H]7c8ccc(O)cc8CC[C@]7(F)c7ccccc7)cc6)CC5)CC4)ccc3C2=O)C(=O)N1] |
| I-167‡ | 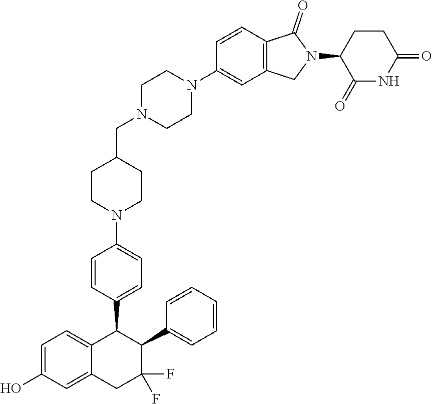 [SMILES: O=C1CC[C@H](N2Cc3cc(N4CCN(CC5CCN(c6ccc([C@@H]7c8ccc(O)cc8CC(F)(F)[C@@H]7c7ccccc7)cc6)CC5)CC4)ccc3C2=O)C(=O)N1] |
| I-168‡ | 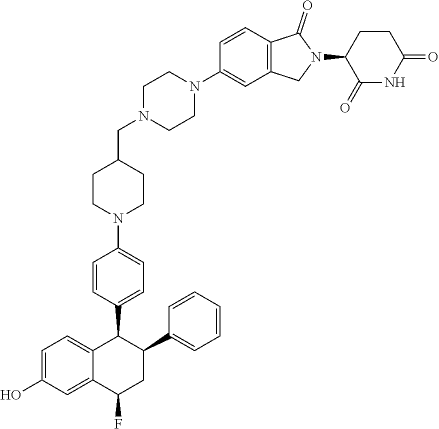 [SMILES: O=C1CC[C@H](N2Cc3cc(N4CCN(CC5CCN(c6ccc([C@@H]7c8ccc(O)cc8[C@H](F)C[C@@H]7c7ccccc7)cc6)CC5)CC4)ccc3C2=O)C(=O)N1] |
| I-169‡ | 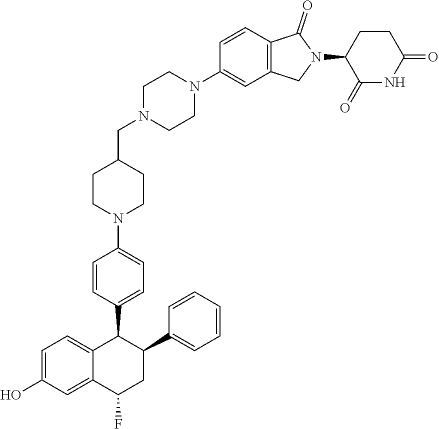 [SMILES: O=C1CC[C@H](N2Cc3cc(N4CCN(CC5CCN(c6ccc([C@@H]7c8ccc(O)cc8[C@@H](F)C[C@@H]7c7ccccc7)cc6)CC5)CC4)ccc3C2=O)C(=O)N1] |
| I-170‡ | 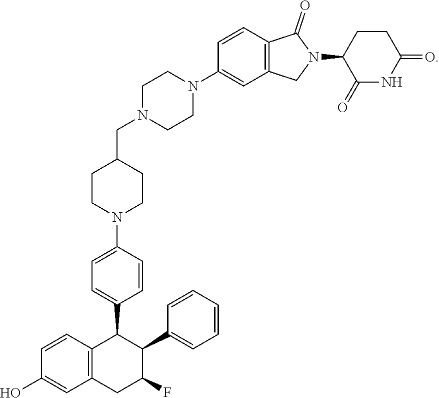 [SMILES: O=C1CC[C@H](N2Cc3cc(N4CCN(CC5CCN(c6ccc([C@@H]7c8ccc(O)cc8C[C@H](F)[C@@H]7c7ccccc7)cc6)CC5)CC4)ccc3C2=O)C(=O)N1] |
| I-171‡ | 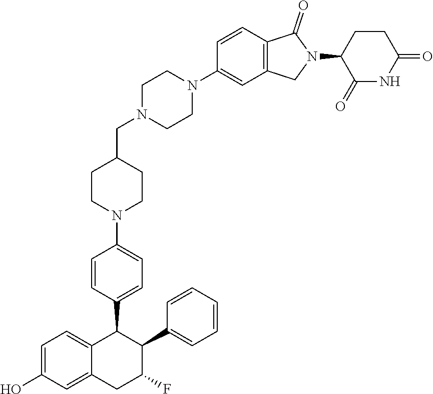 [SMILES: O=C1CC[C@H](N2Cc3cc(N4CCN(CC5CCN(c6ccc([C@@H]7c8ccc(O)cc8C[C@@H](F)[C@@H]7c7ccccc7)cc6)CC5)CC4)ccc3C2=O)C(=O)N1] |
| I-172‡ | 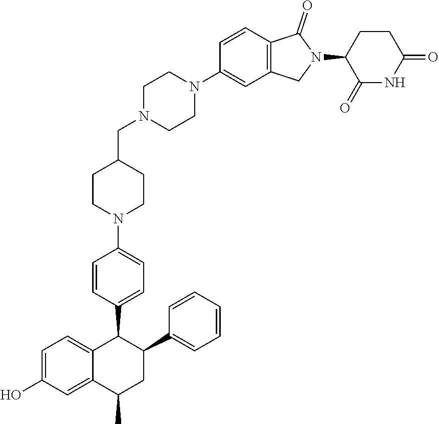 [SMILES: C[C@@H]1C[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-173‡ | 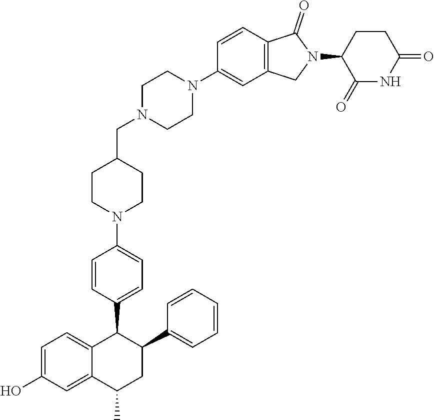 [SMILES: C[C@H]1C[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-174 | 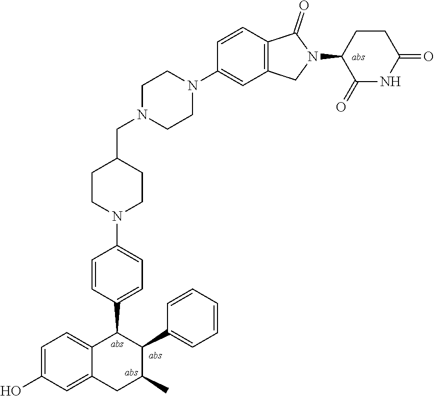 [SMILES: C[C@H]1Cc2cc(O)ccc2[C@@H](c2ccc(N3CCC(CN4CCN(c5ccc6c(c5)CN([C@H]5CCC(=O)NC5=O)C6=O)CC4)CC3)cc2)[C@H]1c1ccccc1] |
| I-175 | 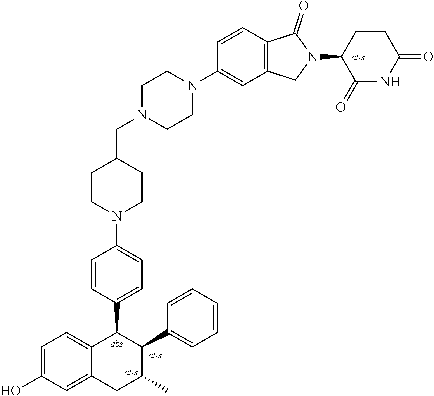 [SMILES: C[C@@H]1Cc2cc(O)ccc2[C@@H](c2ccc(N3CCC(CN4CCN(c5ccc6c(c5)CN([C@H]5CCC(=O)NC5=O)C6=O)CC4)CC3)cc2)[C@H]1c1ccccc1] |
| I-176 | 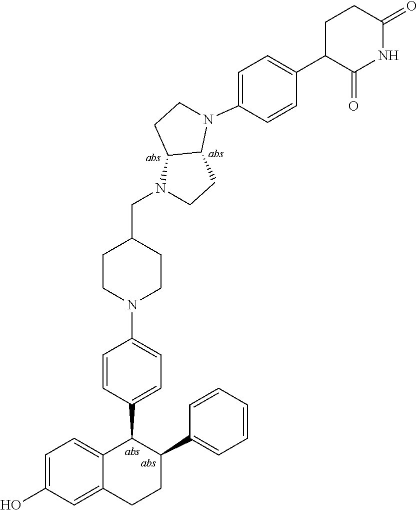 [SMILES: O=C1CCC(c2ccc(N3CC[C@@H]4[C@H]3CCN4CC3CCN(c4ccc([C@@H]5c6ccc(O)cc6CC[C@@H]5c5ccccc5)cc4)CC3)cc2)C(=O)N1] |
| I-177 | 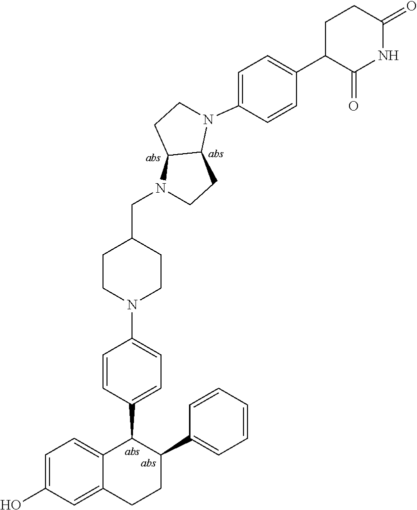 [SMILES: O=C1CCC(c2ccc(N3CC[C@H]4[C@@H]3CCN4CC3CCN(c4ccc([C@@H]5c6ccc(O)cc6CC[C@@H]5c5ccccc5)cc4)CC3)cc2)C(=O)N1] |
| I-178 | 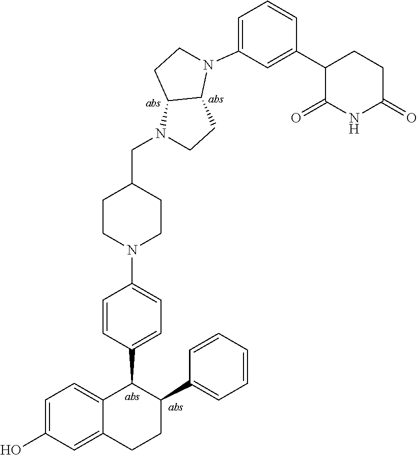 [SMILES: O=C1CCC(c2cccc(N3CC[C@@H]4[C@H]3CCN4CC3CCN(c4ccc([C@@H]5c6ccc(O)cc6CC[C@@H]5c5ccccc5)cc4)CC3)c2)C(=O)N1] |
| I-179 | 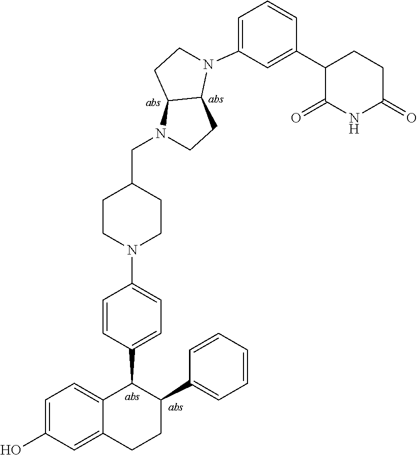 [SMILES: O=C1CCC(c2cccc(N3CC[C@H]4[C@@H]3CCN4CC3CCN(c4ccc([C@@H]5c6ccc(O)cc6CC[C@@H]5c5ccccc5)cc4)CC3)c2)C(=O)N1] |
| I-180 | 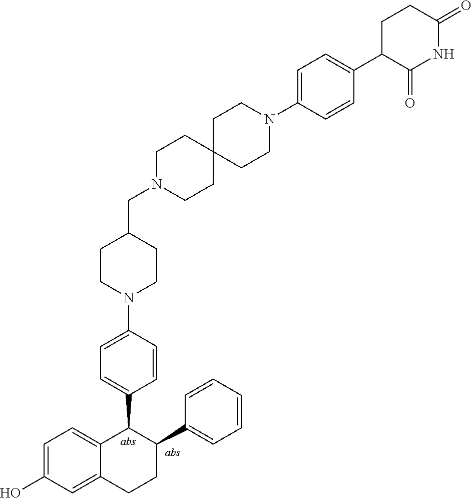 [SMILES: O=C1CCC(c2ccc(N3CCC4(CCN(CC5CCN(c6ccc([C@@H]7c8ccc(O)cc8CC[C@@H]7c7ccccc7)cc6)CC5)CC4)CC3)cc2)C(=O)N1] |
| I-181‡ | 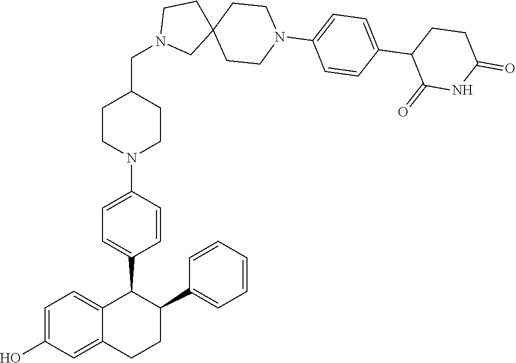 [SMILES: O=C1CCC(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1] |
| I-182 | 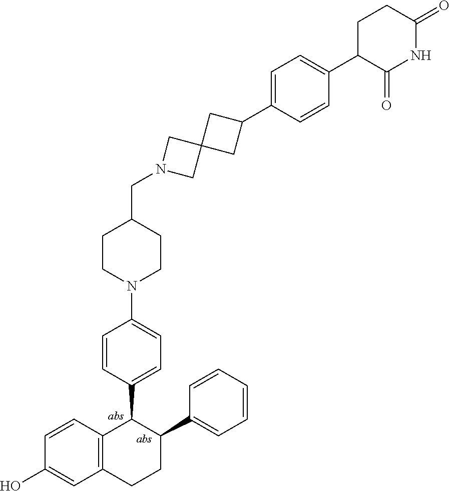 [SMILES: O=C1CCC(c2ccc(C3CC4(C3)CN(CC3CCN(c5ccc([C@@H]6c7ccc(O)cc7CC[C@@H]6c6ccccc6)cc5)CC3)C4)cc2)C(=O)N1] |
| I-183 | 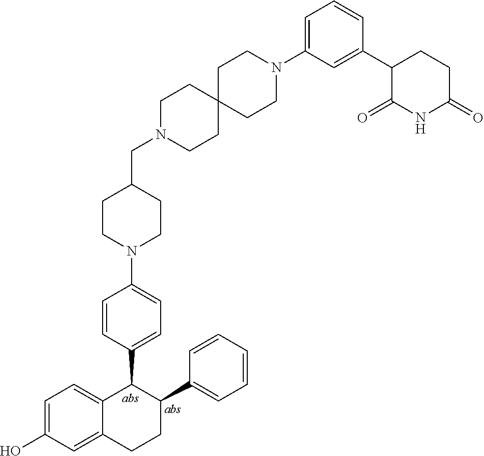 [SMILES: O=C1CCC(c2cccc(N3CCC4(CCN(CC5CCN(c6ccc([C@@H]7c8ccc(O)cc8CC[C@@H]7c7ccccc7)cc6)CC5)CC4)CC3)c2)C(=O)N1] |
| I-184 | 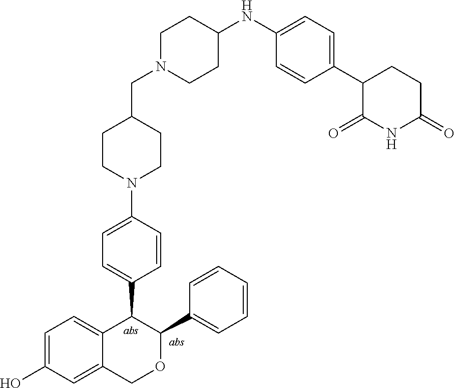 [SMILES: O=C1CCC(c2ccc(NC3CCN(CC4CCN(c5ccc([C@@H]6c7ccc(O)cc7CO[C@@H]6c6ccccc6)cc5)CC4)CC3)cc2)C(=O)N1] |
| I-185 | 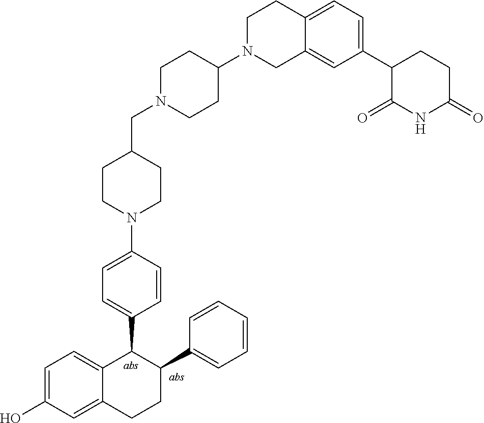 [SMILES: O=C1CCC(c2ccc3c(c2)CN(C2CCN(CC4CCN(c5ccc([C@@H]6c7ccc(O)cc7CC[C@@H]6c6ccccc6)cc5)CC4)CC2)CC3)C(=O)N1] |
| I-186‡ | 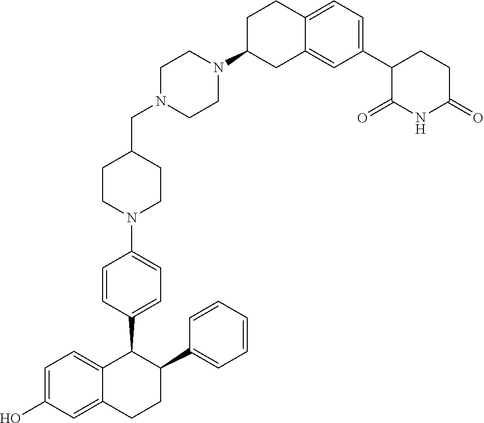 [SMILES: O=C1CCC(c2ccc3c(c2)C[C@@H](N2CCN(CC4CCN(c5ccc([C@@H]6c7ccc(O)cc7CC[C@@H]6c6ccccc6)cc5)CC4)CC2)CC3)C(=O)N1] |
| I-187‡ | 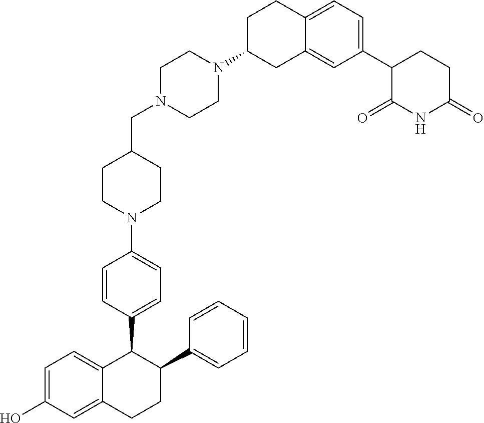 [SMILES: O=C1CCC(c2ccc3c(c2)C[C@H](N2CCN(CC4CCN(c5ccc([C@@H]6c7ccc(O)cc7CC[C@@H]6c6ccccc6)cc5)CC4)CC2)CC3)C(=O)N1] |
| I-188 | 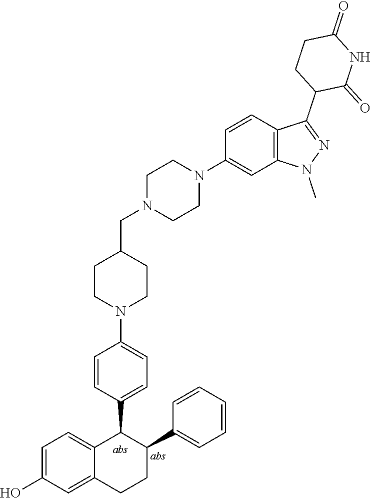 [SMILES: Cn1nc(C2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-189 | 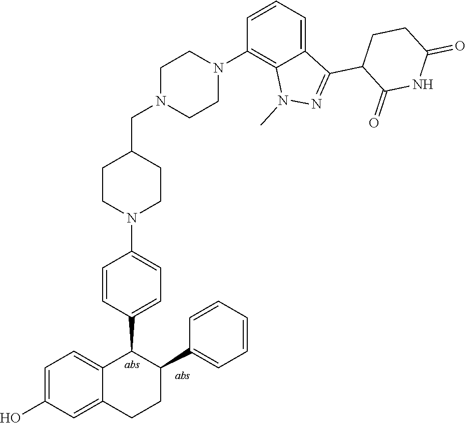 [SMILES: Cn1nc(C2CCC(=O)NC2=O)c2cccc(N3CCN(CC4CCN(c5ccc([C@@H]6c7ccc(O)cc7CC[C@@H]6c6ccccc6)cc5)CC4)CC3)c21] |
| I-190 | 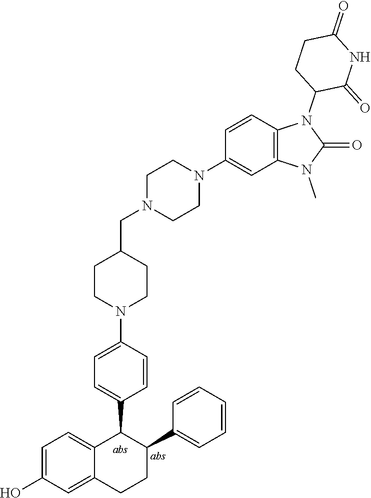 [SMILES: Cn1c(=O)n(C2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-191 | 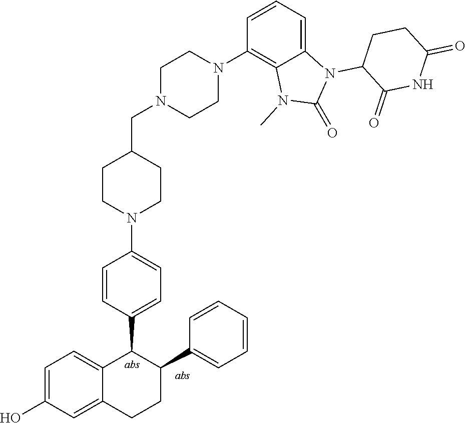 [SMILES: Cn1c(=O)n(C2CCC(=O)NC2=O)c2cccc(N3CCN(CC4CCN(c5ccc([C@@H]6c7ccc(O)cc7CC[C@@H]6c6ccccc6)cc5)CC4)CC3)c21] |
| I-192 | 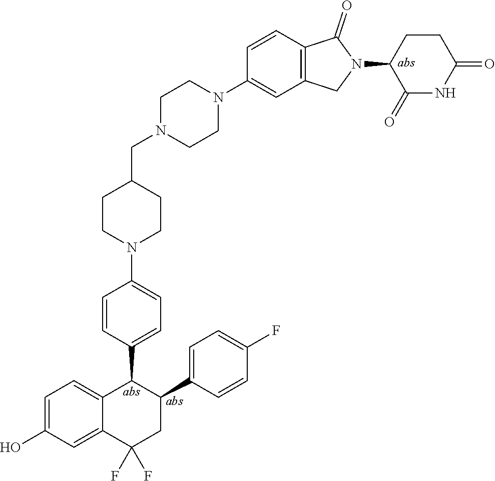 [SMILES: O=C1CC[C@H](N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1] |
| I-193‡ | 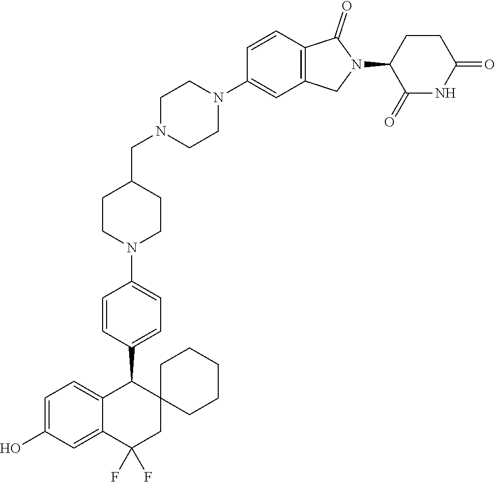 [SMILES: O=C1CC[C@H](N2Cc3cc(N4CCN(CC5CCN(c6ccc([C@@H]7c8ccc(O)cc8C(F)(F)CC78CCCCC8)cc6)CC5)CC4)ccc3C2=O)C(=O)N1] |
| I-194 | 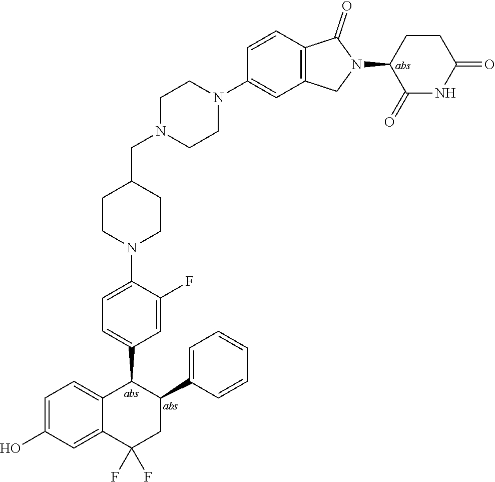 [SMILES: O=C1CC[C@H](N2Cc3cc(N4CCN(CC5CCN(c6ccc([C@@H]7c8ccc(O)cc8C(F)(F)C[C@@H]7c7ccccc7)cc6F)CC5)CC4)ccc3C2=O)C(=O)N1] |
| I-195‡ | 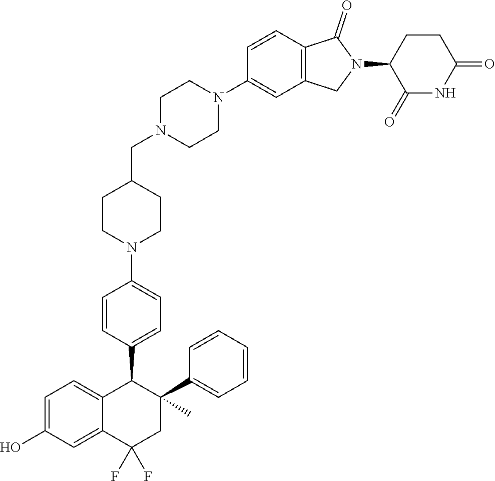 [SMILES: C[C@]1(c2ccccc2)CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-196 | 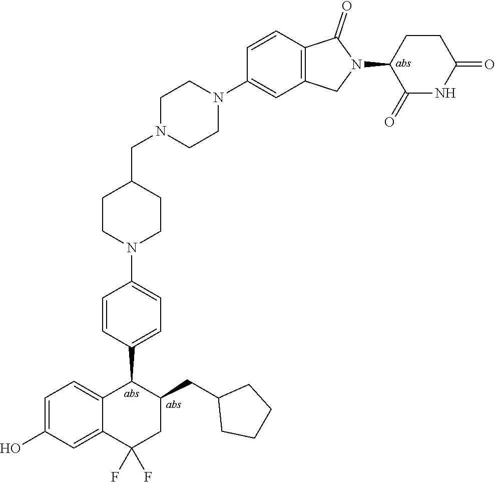 [SMILES: O=C1CC[C@H](N2Cc3cc(N4CCN(CC5CCN(c6ccc([C@@H]7c8ccc(O)cc8C(F)(F)C[C@@H]7CC7CCCC7)cc6)CC5)CC4)ccc3C2=O)C(=O)N1] |
| I-197 | 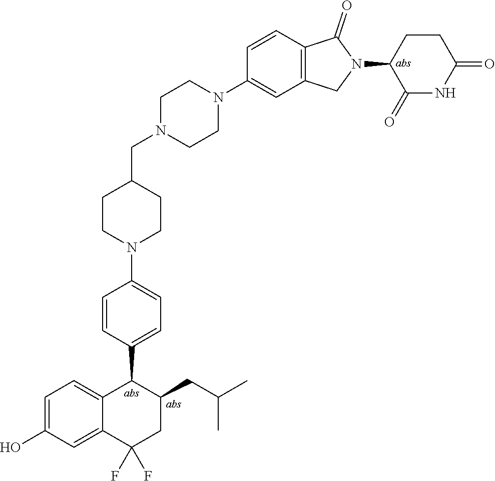 [SMILES: CC(C)C[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-198 | 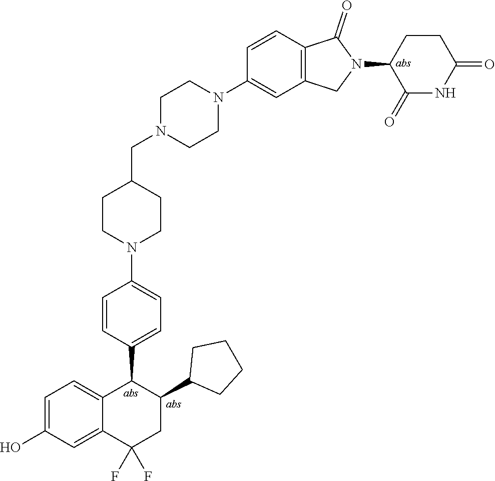 [SMILES: O=C1CC[C@H](N2Cc3cc(N4CCN(CC5CCN(c6ccc([C@@H]7c8ccc(O)cc8C(F)(F)C[C@@H]7C7CCCC7)cc6)CC5)CC4)ccc3C2=O)C(=O)N1] |
| I-199 | 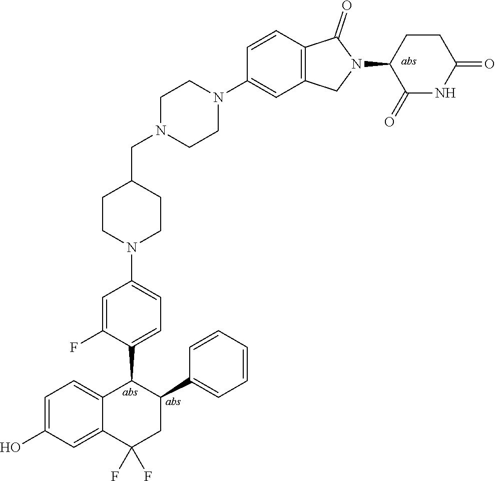 [SMILES: O=C1CC[C@H](N2Cc3cc(N4CCN(CC5CCN(c6ccc([C@@H]7c8ccc(O)cc8C(F)(F)C[C@@H]7c7ccccc7)c(F)c6)CC5)CC4)ccc3C2=O)C(=O)N1] |
| I-200‡ | 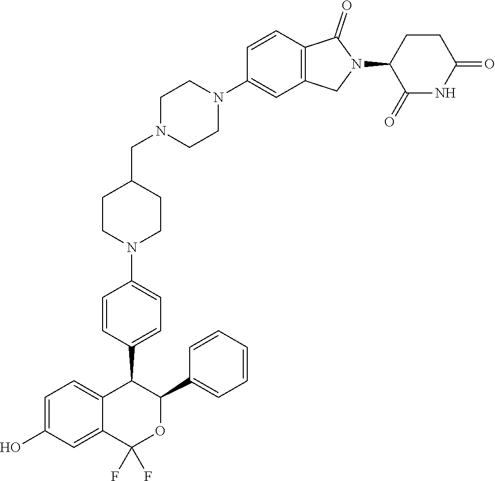 [SMILES: O=C1CC[C@H](N2Cc3cc(N4CCN(CC5CCN(c6ccc([C@@H]7c8ccc(O)cc8C(F)(F)O[C@@H]7c7ccccc7)cc6)CC5)CC4)ccc3C2=O)C(=O)N1] |
| I-201‡ | 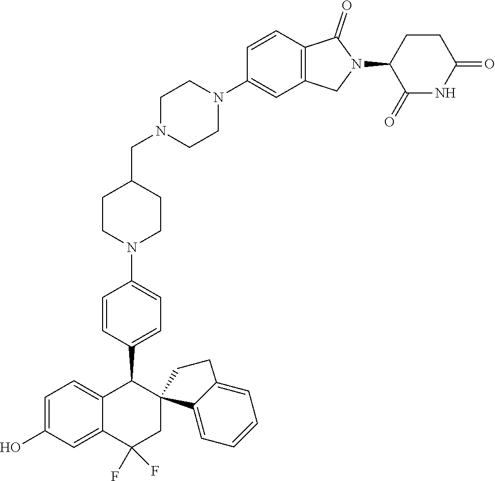 [SMILES: O=C1CC[C@H](N2Cc3cc(N4CCN(CC5CCN(c6ccc([C@@H]7c8ccc(O)cc8C(F)(F)C[C@@]78CCc7ccccc78)cc6)CC5)CC4)ccc3C2=O)C(=O)N1] |
| I-202 | 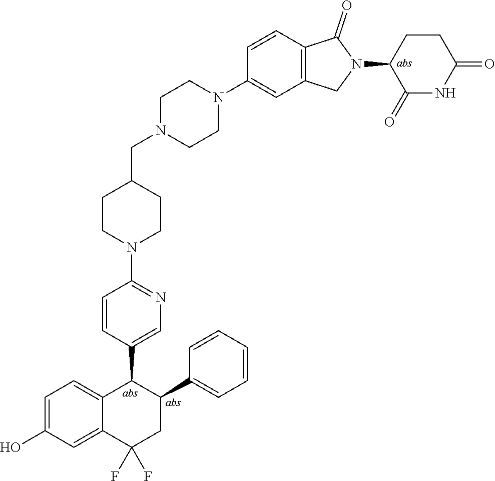 [SMILES: O=C1CC[C@H](N2Cc3cc(N4CCN(CC5CCN(c6ccc([C@@H]7c8ccc(O)cc8C(F)(F)C[C@@H]7c7ccccc7)cn6)CC5)CC4)ccc3C2=O)C(=O)N1] |
| I-203 | 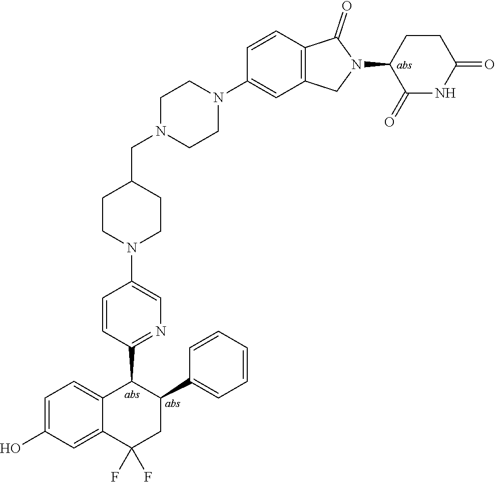 [SMILES: O=C1CC[C@H](N2Cc3cc(N4CCN(CC5CCN(c6ccc([C@@H]7c8ccc(O)cc8C(F)(F)C[C@@H]7c7ccccc7)nc6)CC5)CC4)ccc3C2=O)C(=O)N1] |
| I-204 | 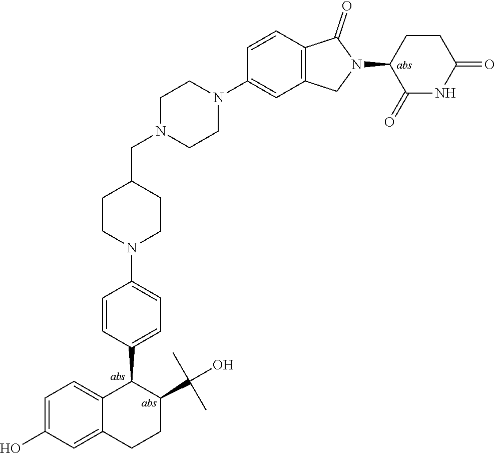 [SMILES: CC(C)(O)[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-205 | 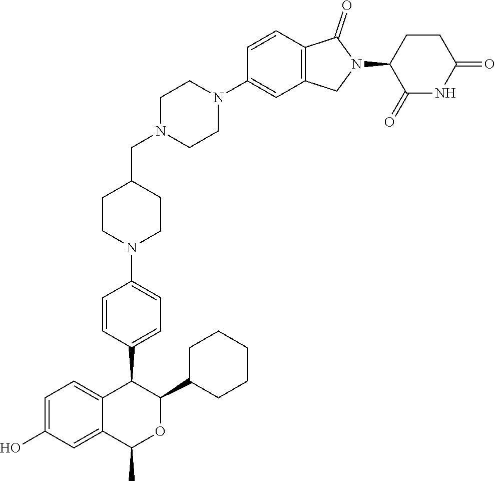 [SMILES: C[C@@H]1O[C@H](C2CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-206‡ | 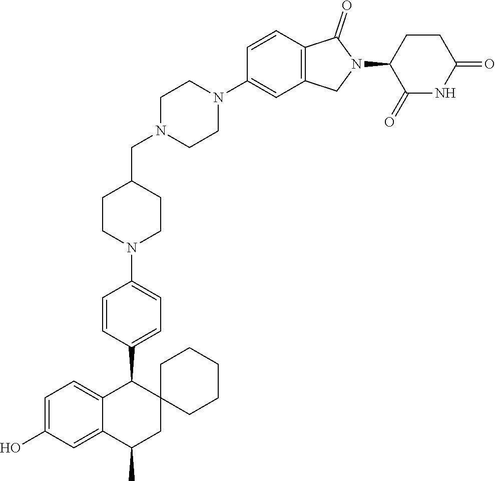 [SMILES: C[C@@H]1CC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-207‡ | 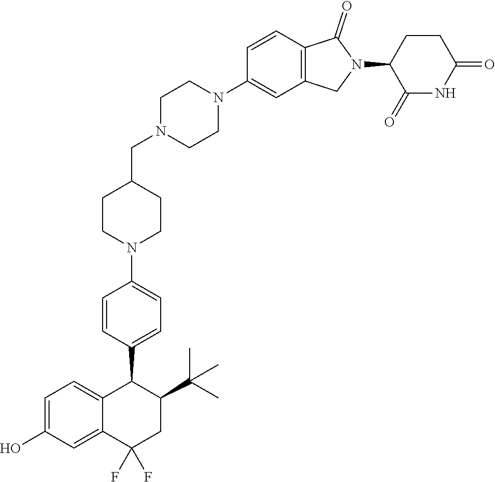 [SMILES: CC(C)(C)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-208‡ | 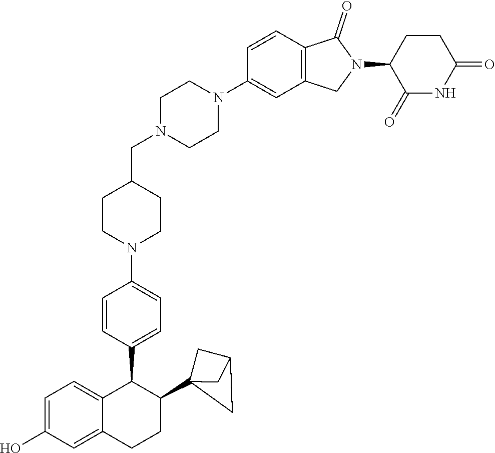 [SMILES: O=C1CC[C@H](N2Cc3cc(N4CCN(CC5CCN(c6ccc([C@@H]7c8ccc(O)cc8CC[C@@H]7C78CC(C7)C8)cc6)CC5)CC4)ccc3C2=O)C(=O)N1] |
| I-209‡ | 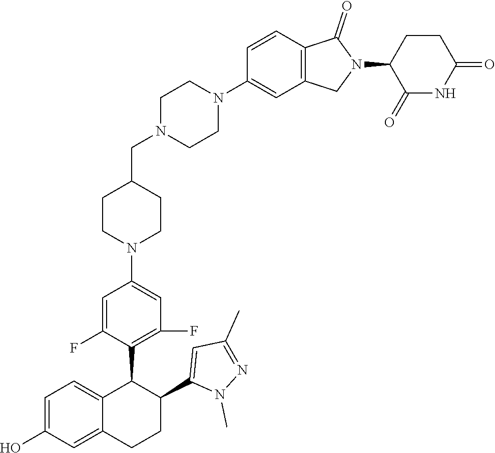 [SMILES: Cc1cc([C@H]2CCc3cc(O)ccc3[C@H]2c2c(F)cc(N3CCC(CN4CCN(c5ccc6c(c5)CN([C@H]5CCC(=O)NC5=O)C6=O)CC4)CC3)cc2F)n(C)n1] |
| I-210‡ | 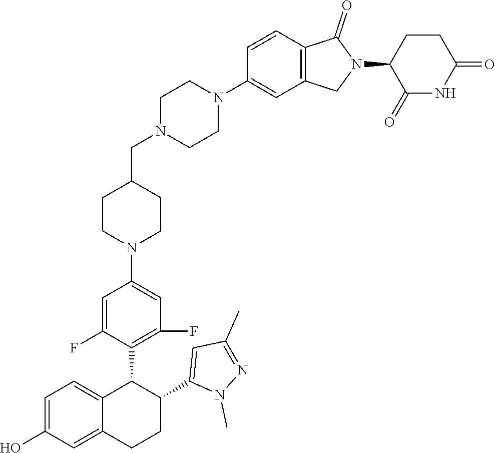 [SMILES: Cc1cc([C@@H]2CCc3cc(O)ccc3[C@@H]2c2c(F)cc(N3CCC(CN4CCN(c5ccc6c(c5)CN([C@H]5CCC(=O)NC5=O)C6=O)CC4)CC3)cc2F)n(C)n1] |
| I-211 | 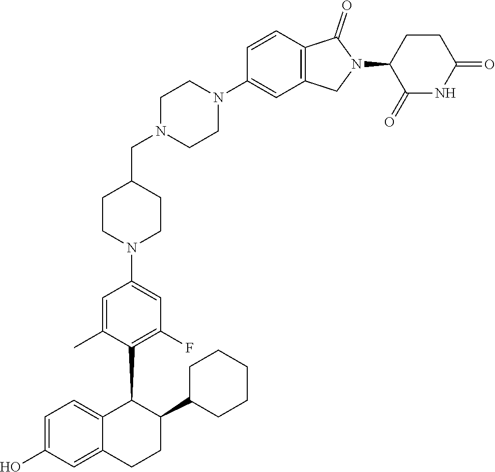 [SMILES: Cc1cc(N2CCC(CN3CCN(c4ccc5c(c4)CN([C@H]4CCC(=O)NC4=O)C5=O)CC3)CC2)cc(F)c1[C@@H]1c2ccc(O)cc2CC[C@@H]1C1CCCCC1] |
| I-212‡ | 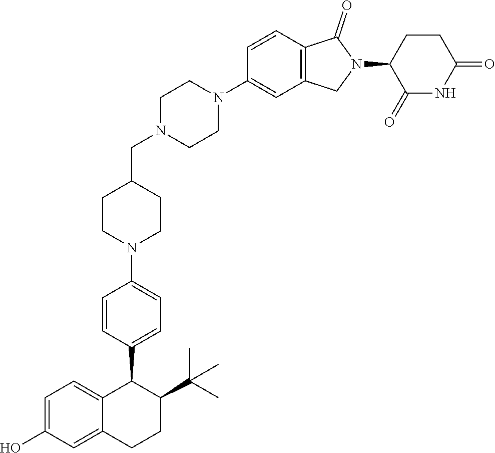 [SMILES: CC(C)(C)[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-213‡ | 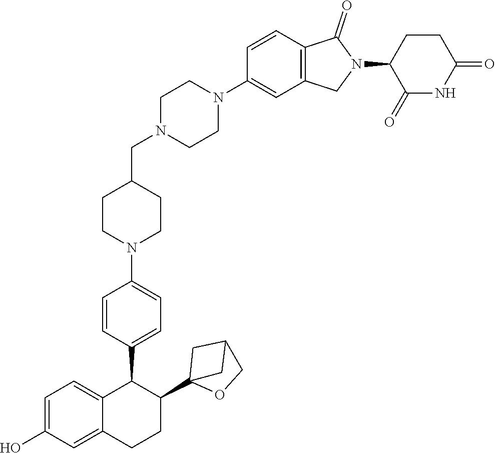 [SMILES: O=C1CC[C@H](N2Cc3cc(N4CCN(CC5CCN(c6ccc([C@@H]7c8ccc(O)cc8CC[C@@H]7C78CC(CO7)C8)cc6)CC5)CC4)ccc3C2=O)C(=O)N1] |
| I-214‡ | 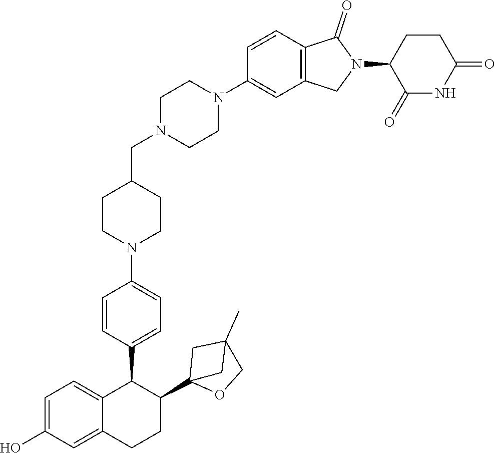 [SMILES: CC12COC([C@H]3CCc4cc(O)ccc4[C@H]3c3ccc(N4CCC(CN5CCN(c6ccc7c(c6)CN([C@H]6CCC(=O)NC6=O)C7=O)CC5)CC4)cc3)(C1)C2] |
| I-215‡ | 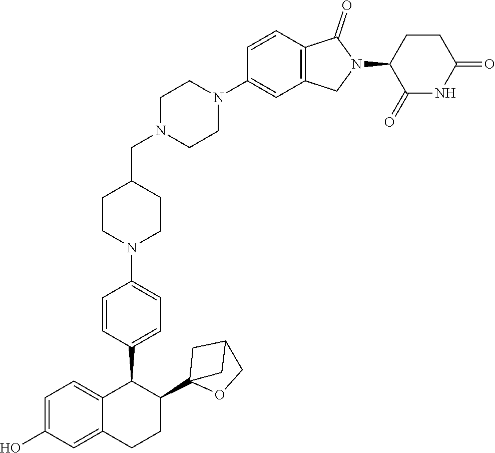 [SMILES: O=C1CC[C@H](N2Cc3cc(N4CCN(CC5CCN(c6ccc([C@@H]7c8ccc(O)cc8CC[C@@H]7C78CC(CO7)C8)cc6)CC5)CC4)ccc3C2=O)C(=O)N1] |
| I-216‡ | 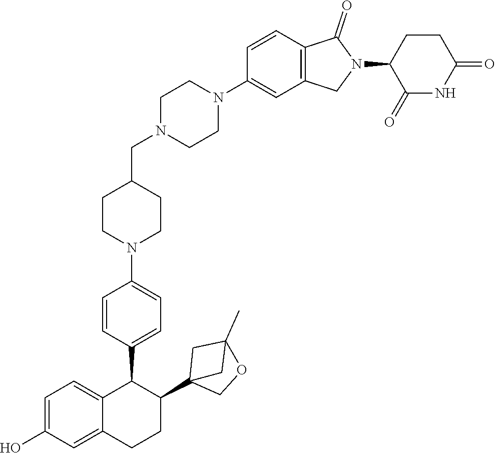 [SMILES: CC12CC([C@H]3CCc4cc(O)ccc4[C@H]3c3ccc(N4CCC(CN5CCN(c6ccc7c(c6)CN([C@H]6CCC(=O)NC6=O)C7=O)CC5)CC4)cc3)(CO1)C2] |
| I-219 | 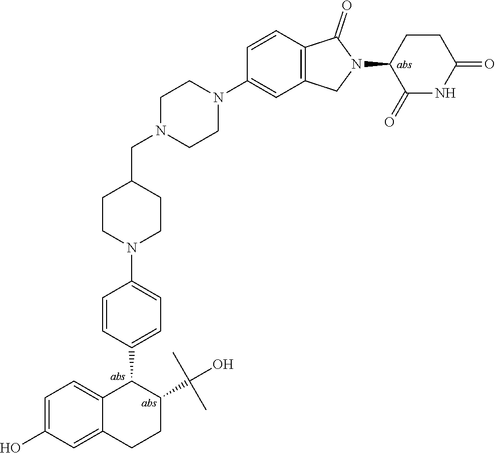 [SMILES: CC(C)(O)[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-220 | 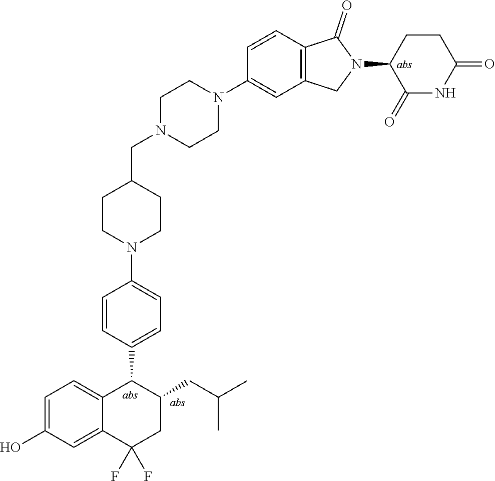 [SMILES: CC(C)C[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-221 | 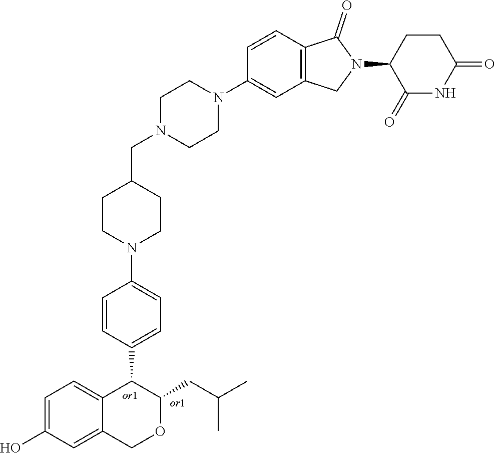 [SMILES: CC(C)C[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-222 | 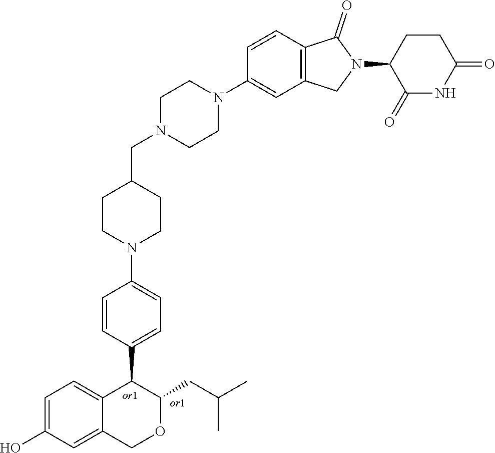 [SMILES: CC(C)C[C@@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-223 | 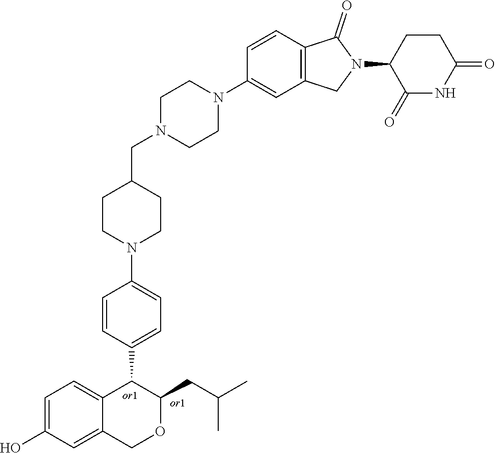 [SMILES: CC(C)C[C@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-224 | 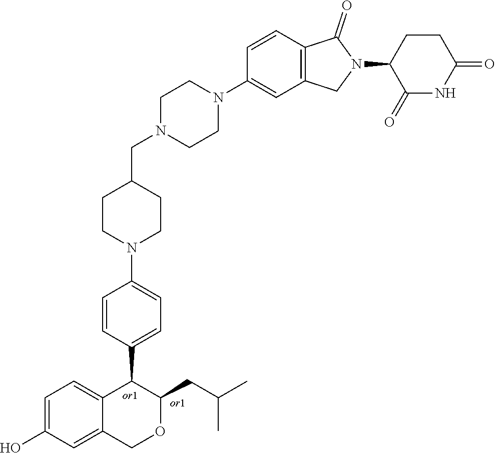 [SMILES: CC(C)C[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-225 | 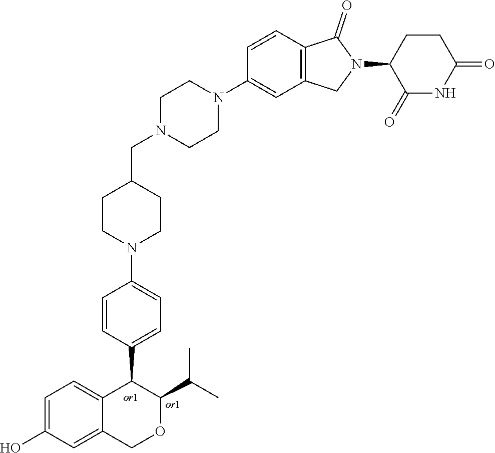 [SMILES: CC(C)[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-226 | 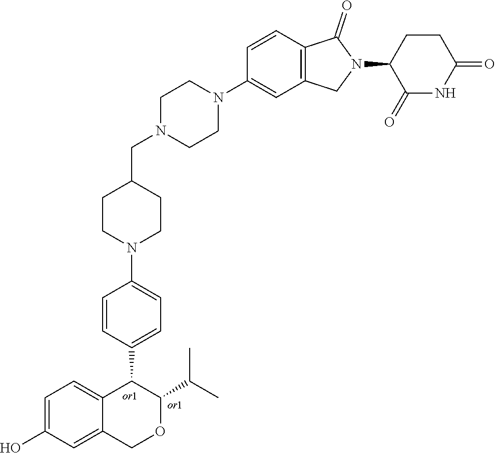 [SMILES: CC(C)[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-227 | 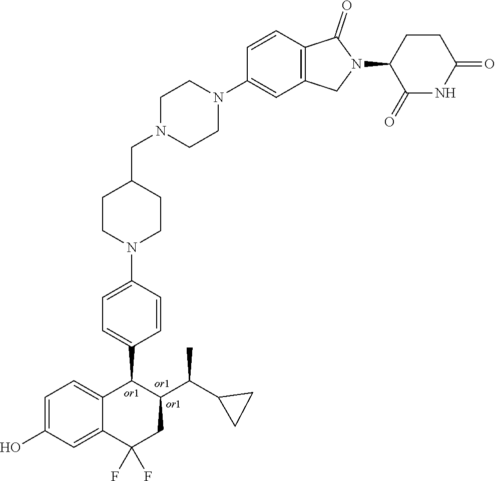 [SMILES: C[C@@H](C1CC1)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-228 | 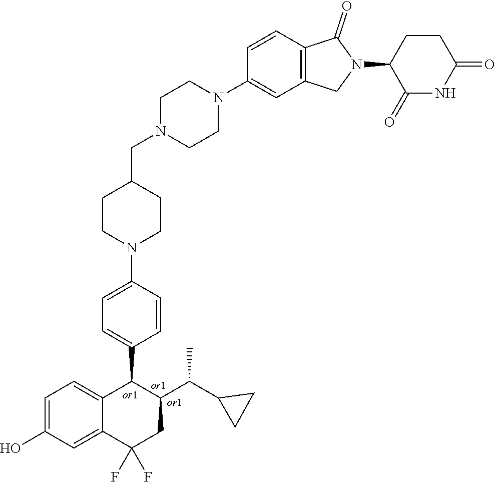 [SMILES: C[C@H](C1CC1)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-229 | 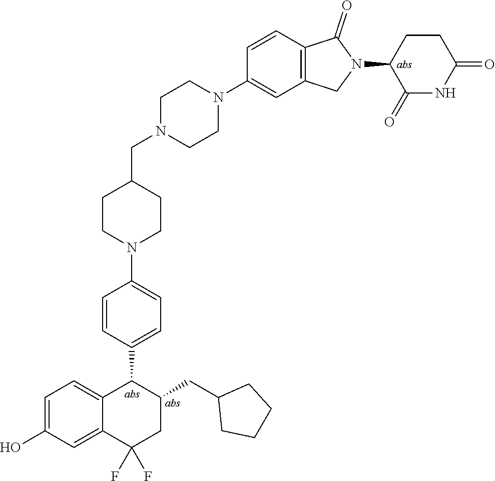 [SMILES: O=C1CC[C@H](N2Cc3cc(N4CCN(CC5CCN(c6ccc([C@H]7c8ccc(O)cc8C(F)(F)C[C@H]7CC7CCCC7)cc6)CC5)CC4)ccc3C2=O)C(=O)N1] |
| I-230 | 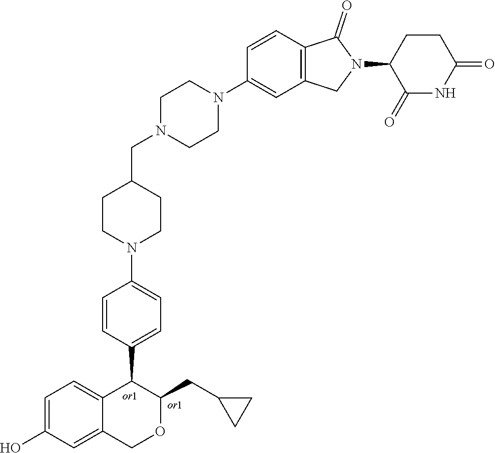 [SMILES: O=C1CC[C@H](N2Cc3cc(N4CCN(CC5CCN(c6ccc([C@@H]7c8ccc(O)cc8CO[C@@H]7CC7CC7)cc6)CC5)CC4)ccc3C2=O)C(=O)N1] |
| I-231 | 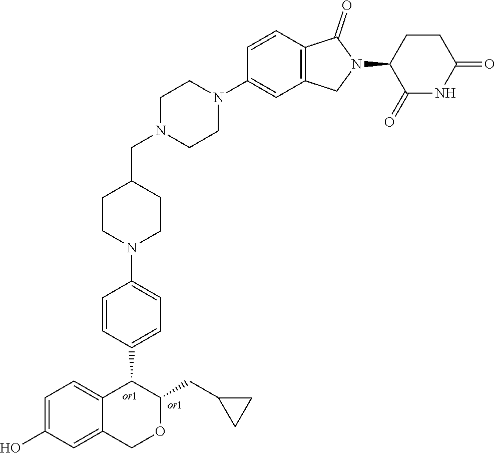 [SMILES: O=C1CC[C@H](N2Cc3cc(N4CCN(CC5CCN(c6ccc([C@H]7c8ccc(O)cc8CO[C@H]7CC7CC7)cc6)CC5)CC4)ccc3C2=O)C(=O)N1] |
| I-232 | 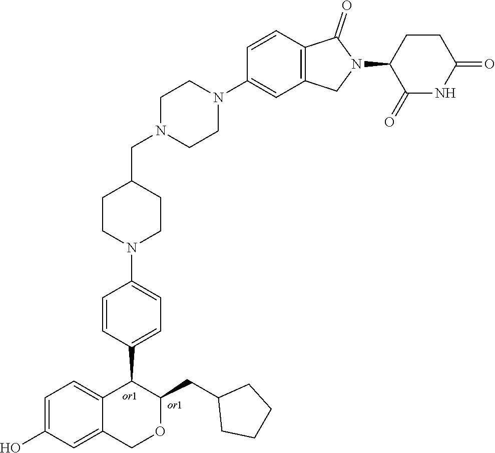 [SMILES: O=C1CC[C@H](N2Cc3cc(N4CCN(CC5CCN(c6ccc([C@@H]7c8ccc(O)cc8CO[C@@H]7CC7CCCC7)cc6)CC5)CC4)ccc3C2=O)C(=O)N1] |
| I-233 | 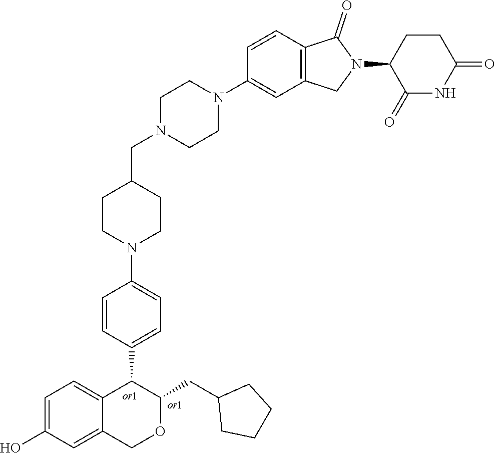 [SMILES: O=C1CC[C@H](N2Cc3cc(N4CCN(CC5CCN(c6ccc([C@H]7c8ccc(O)cc8CO[C@H]7CC7CCCC7)cc6)CC5)CC4)ccc3C2=O)C(=O)N1] |
| I-234 | 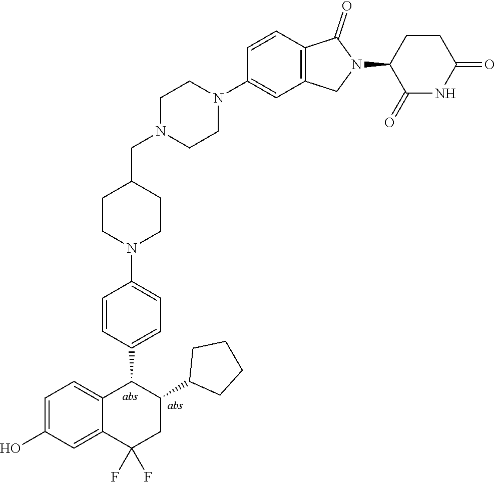 [SMILES: O=C1CC[C@H](N2Cc3cc(N4CCN(CC5CCN(c6ccc([C@H]7c8ccc(O)cc8C(F)(F)C[C@H]7C7CCCC7)cc6)CC5)CC4)ccc3C2=O)C(=O)N1] |
| I-235 | 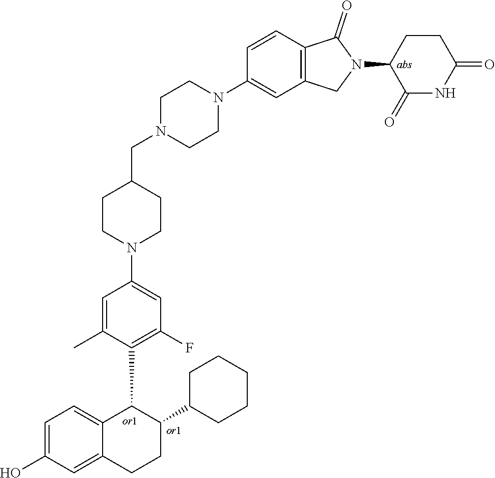 [SMILES: Cc1cc(N2CCC(CN3CCN(c4ccc5c(c4)CN([C@H]4CCC(=O)NC4=O)C5=O)CC3)CC2)cc(F)c1[C@H]1c2ccc(O)cc2CC[C@H]1C1CCCCC1] |
| I-236 | 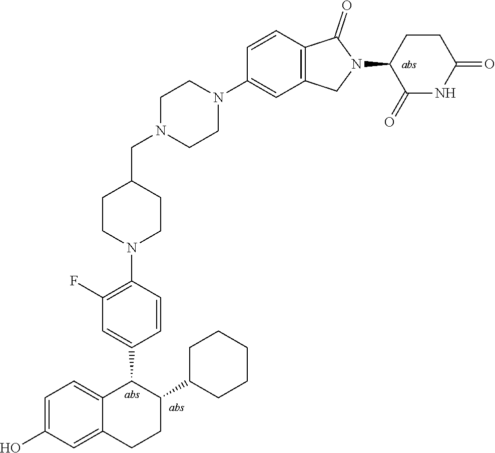 [SMILES: O=C1CC[C@H](N2Cc3cc(N4CCN(CC5CCN(c6ccc([C@H]7c8ccc(O)cc8CC[C@H]7C7CCCCC7)cc6F)CC5)CC4)ccc3C2=O)C(=O)N1] |
| I-237 | 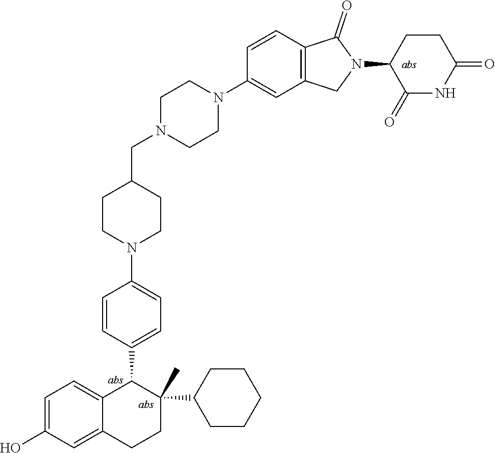 [SMILES: C[C@@]1(C2CCCCC2)CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-238 | 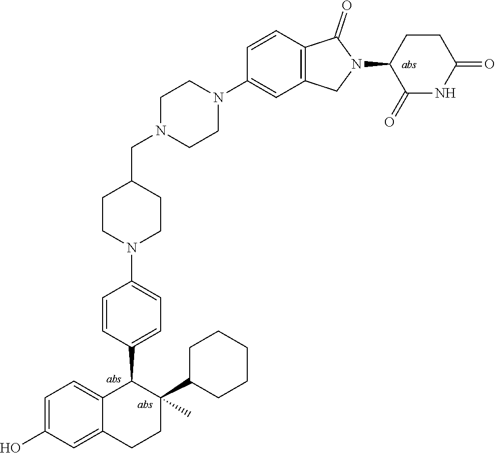 [SMILES: C[C@]1(C2CCCCC2)CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-239 | 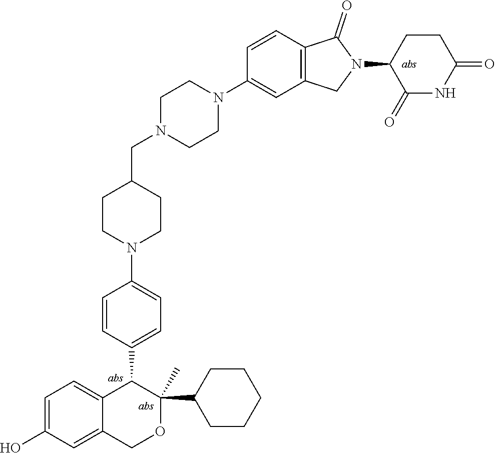 [SMILES: C[C@]1(C2CCCCC2)OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-240 | 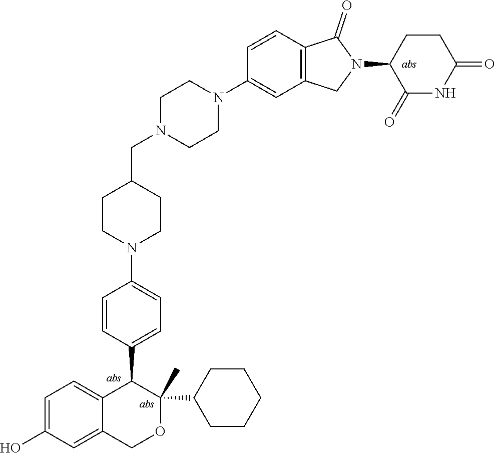 [SMILES: C[C@@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-241 | 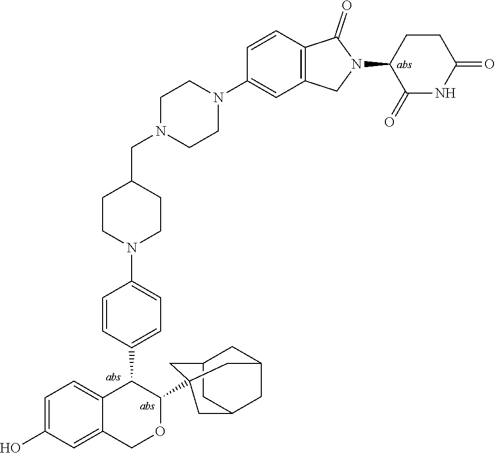 [SMILES: O=C1CC[C@H](N2Cc3cc(N4CCN(CC5CCN(c6ccc([C@H]7c8ccc(O)cc8CO[C@H]7C78CC9CC(CC(C9)C7)C8)cc6)CC5)CC4)ccc3C2=O)C(=O)N1] |
| I-242 | 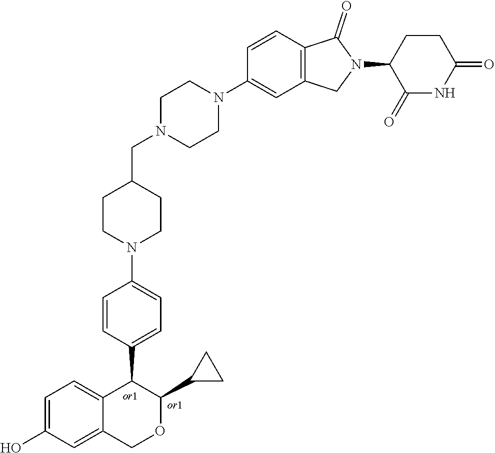 [SMILES: O=C1CC[C@H](N2Cc3cc(N4CCN(CC5CCN(c6ccc([C@@H]7c8ccc(O)cc8CO[C@@H]7C7CC7)cc6)CC5)CC4)ccc3C2=O)C(=O)N1] |
| I-243 | 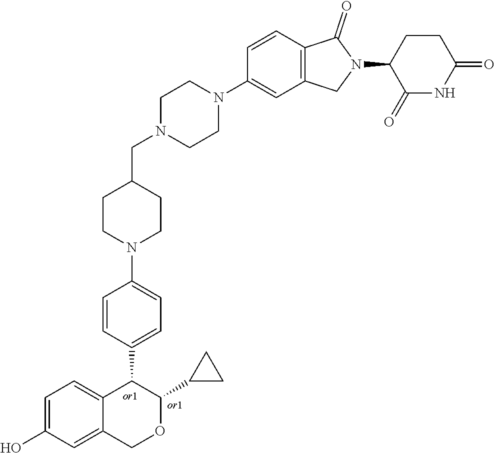 [SMILES: O=C1CC[C@H](N2Cc3cc(N4CCN(CC5CCN(c6ccc([C@H]7c8ccc(O)cc8CO[C@H]7C7CC7)cc6)CC5)CC4)ccc3C2=O)C(=O)N1] |
| I-244 | 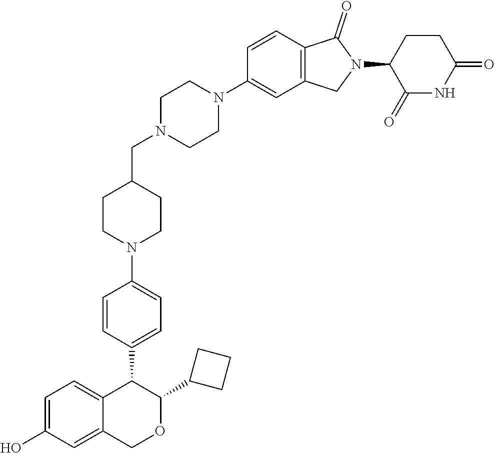 [SMILES: O=C1CC[C@H](N2Cc3cc(N4CCN(CC5CCN(c6ccc([C@H]7c8ccc(O)cc8CO[C@H]7C7CCC7)cc6)CC5)CC4)ccc3C2=O)C(=O)N1] |
| I-245 | 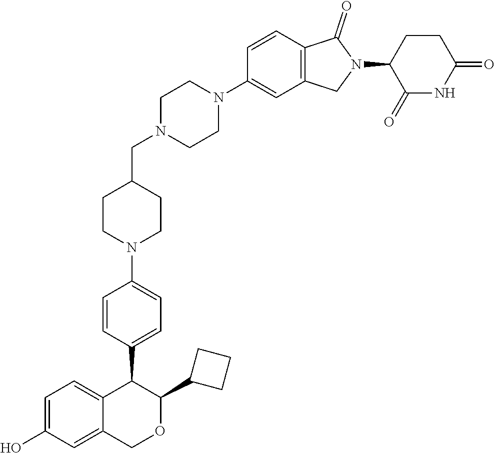 [SMILES: O=C1CC[C@H](N2Cc3cc(N4CCN(CC5CCN(c6ccc([C@@H]7c8ccc(O)cc8CO[C@@H]7C7CCC7)cc6)CC5)CC4)ccc3C2=O)C(=O)N1] |
| I-246 | 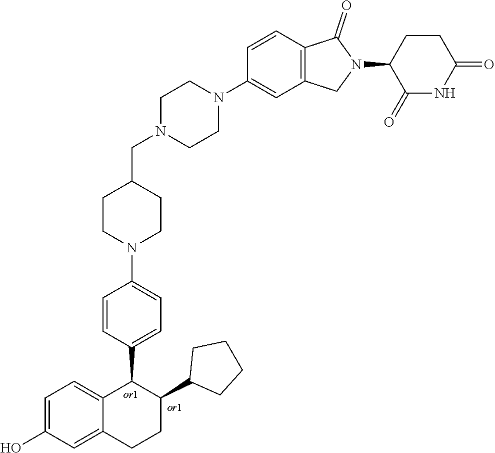 [SMILES: O=C1CC[C@H](N2Cc3cc(N4CCN(CC5CCN(c6ccc([C@@H]7c8ccc(O)cc8CC[C@@H]7C7CCCC7)cc6)CC5)CC4)ccc3C2=O)C(=O)N1] |
| I-247 | 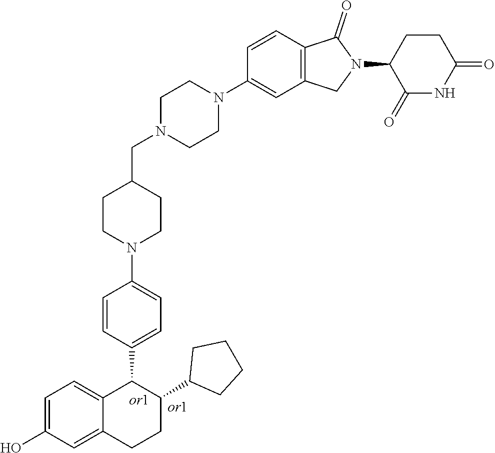 [SMILES: O=C1CC[C@H](N2Cc3cc(N4CCN(CC5CCN(c6ccc([C@H]7c8ccc(O)cc8CC[C@H]7C7CCCC7)cc6)CC5)CC4)ccc3C2=O)C(=O)N1] |
| I-248 | 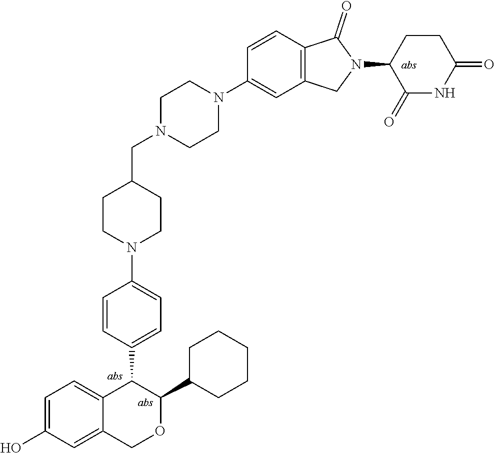 [SMILES: O=C1CC[C@H](N2Cc3cc(N4CCN(CC5CCN(c6ccc([C@H]7c8ccc(O)cc8CO[C@@H]7C7CCCCC7)cc6)CC5)CC4)ccc3C2=O)C(=O)N1] |
| I-249 | 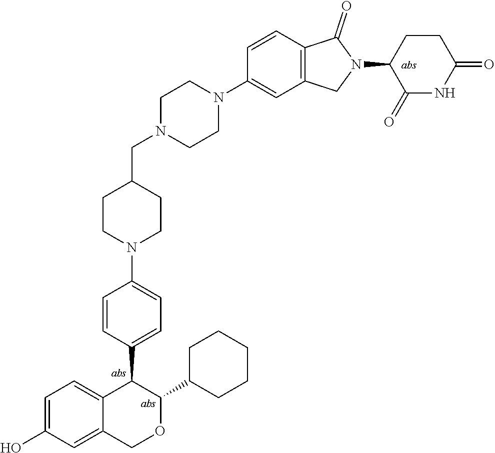 [SMILES: O=C1CC[C@H](N2Cc3cc(N4CCN(CC5CCN(c6ccc([C@@H]7c8ccc(O)cc8CO[C@H]7C7CCCCC7)cc6)CC5)CC4)ccc3C2=O)C(=O)N1] |
| I-250 | 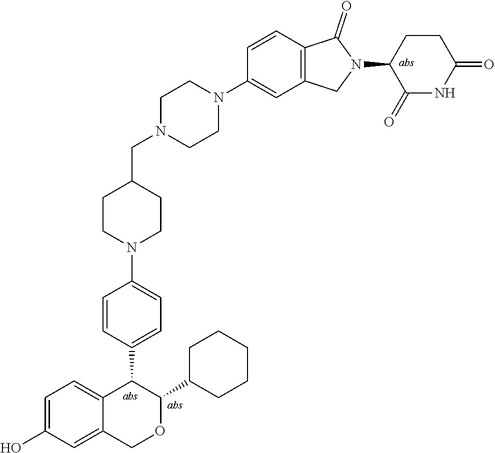 [SMILES: O=C1CC[C@H](N2Cc3cc(N4CCN(CC5CCN(c6ccc([C@H]7c8ccc(O)cc8CO[C@H]7C7CCCCC7)cc6)CC5)CC4)ccc3C2=O)C(=O)N1] |
| I-251 | 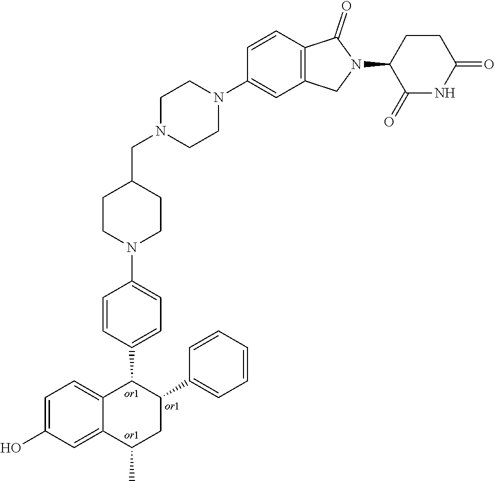 [SMILES: C[C@H]1C[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-252 | 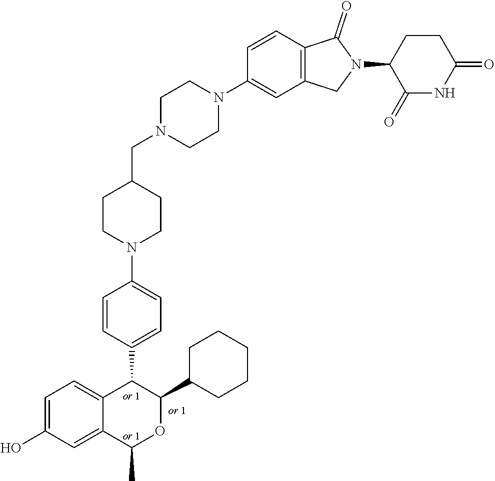 [SMILES: C[C@@H]1O[C@H](C2CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-253 | 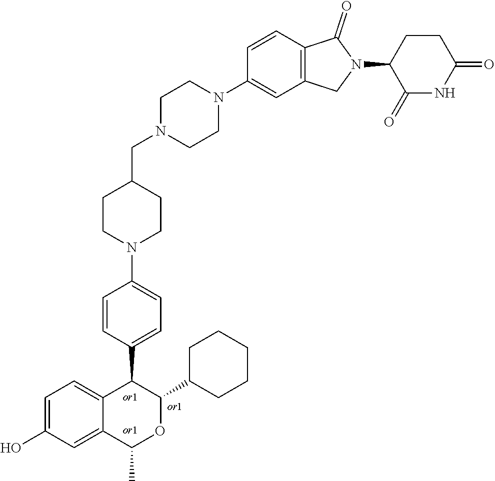 [SMILES: C[C@H]1O[C@@H](C2CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-254 | 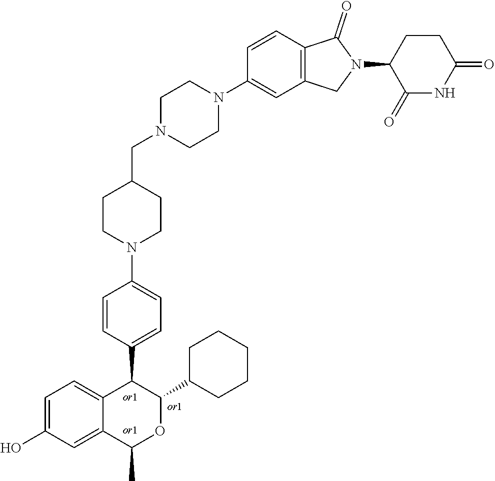 [SMILES: C[C@@H]1O[C@@H](C2CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-255 | 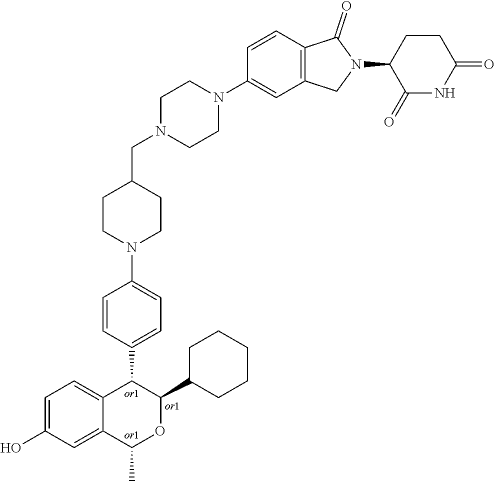 [SMILES: C[C@H]1O[C@H](C2CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-256 | 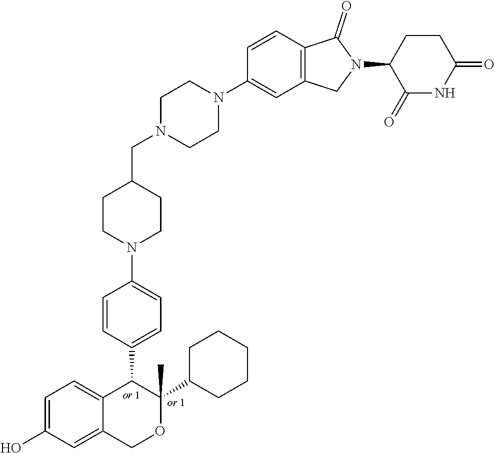 [SMILES: C[C@@]1(C2CCCCC2)OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-257 | 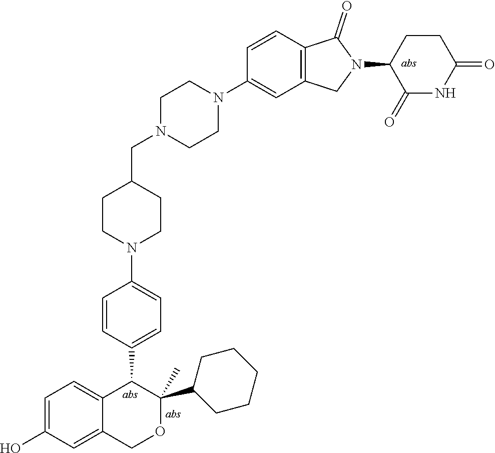 [SMILES: C[C@]1(C2CCCCC2)OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-258 | 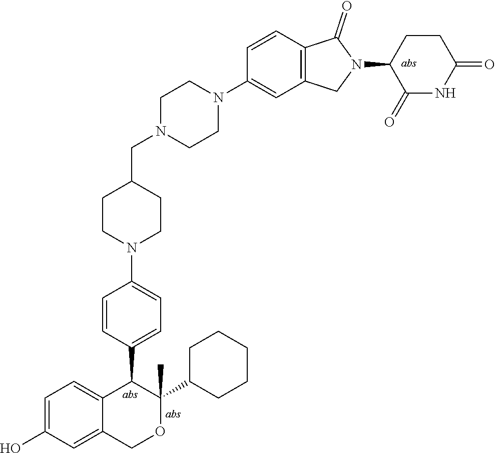 [SMILES: C[C@@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-259 | 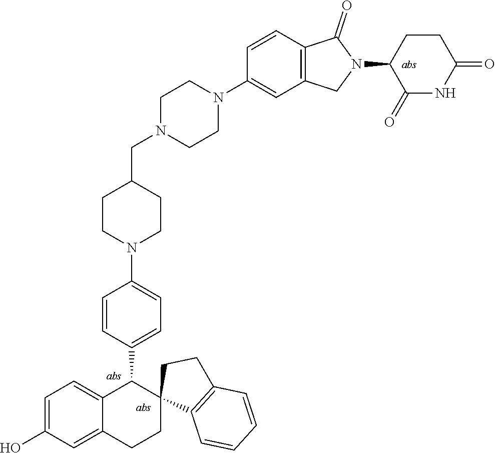 [SMILES: O=C1CC[C@H](N2Cc3cc(N4CCN(CC5CCN(c6ccc([C@H]7c8ccc(O)cc8CC[C@]78CCc7ccccc78)cc6)CC5)CC4)ccc3C2=O)C(=O)N1] |
| I-260 | 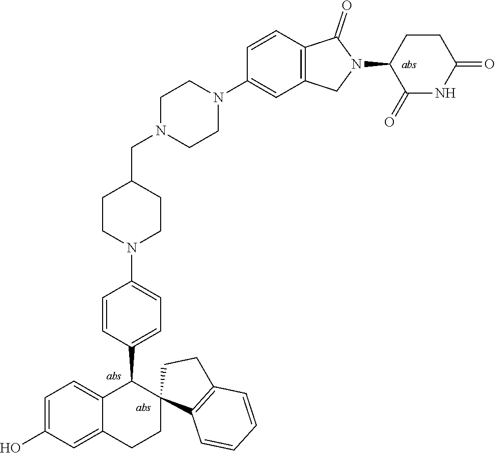 [SMILES: O=C1CC[C@H](N2Cc3cc(N4CCN(CC5CCN(c6ccc([C@@H]7c8ccc(O)cc8CC[C@@]78CCc7ccccc78)cc6)CC5)CC4)ccc3C2=O)C(=O)N1] |
| I-261 | 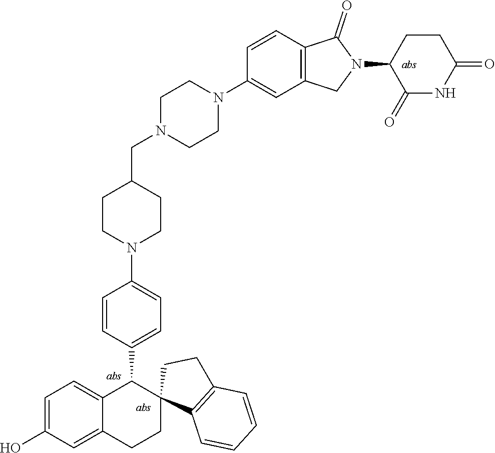 [SMILES: O=C1CC[C@H](N2Cc3cc(N4CCN(CC5CCN(c6ccc([C@H]7c8ccc(O)cc8CC[C@@]78CCc7ccccc78)cc6)CC5)CC4)ccc3C2=O)C(=O)N1] |
| I-262 | 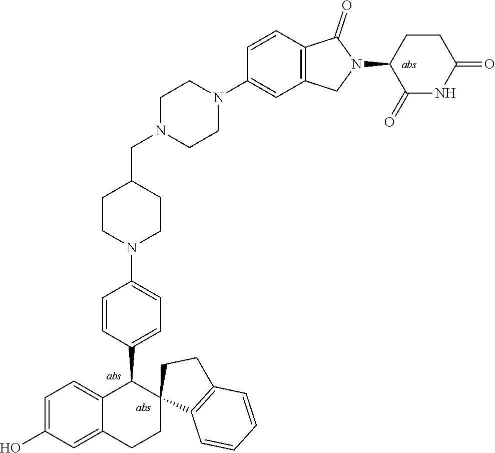 [SMILES: O=C1CC[C@H](N2Cc3cc(N4CCN(CC5CCN(c6ccc([C@@H]7c8ccc(O)cc8CC[C@]78CCc7ccccc78)cc6)CC5)CC4)ccc3C2=O)C(=O)N1] |
| I-263 | 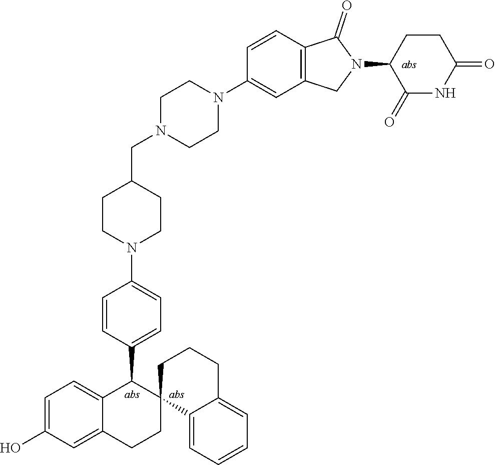 [SMILES: O=C1CC[C@H](N2Cc3cc(N4CCN(CC5CCN(c6ccc([C@@H]7c8ccc(O)cc8CC[C@]78CCCc7ccccc78)cc6)CC5)CC4)ccc3C2=O)C(=O)N1] |
| I-264 | 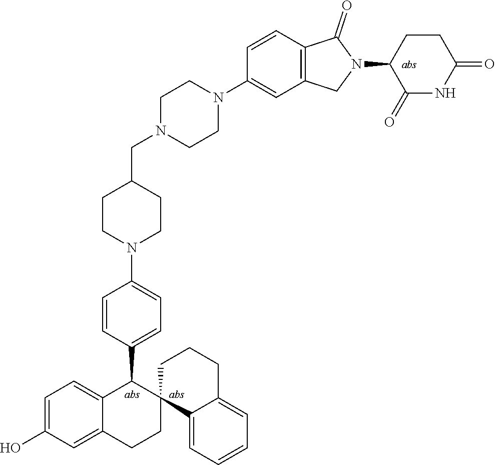 [SMILES: O=C1CC[C@H](N2Cc3cc(N4CCN(CC5CCN(c6ccc([C@@H]7c8ccc(O)cc8CC[C@@]78CCCc7ccccc78)cc6)CC5)CC4)ccc3C2=O)C(=O)N1] |
| I-265 | 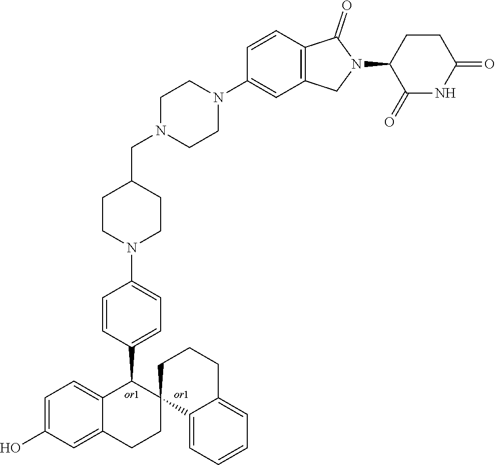 [SMILES: O=C1CC[C@H](N2Cc3cc(N4CCN(CC5CCN(c6ccc([C@@H]7c8ccc(O)cc8CC[C@]78CCCc7ccccc78)cc6)CC5)CC4)ccc3C2=O)C(=O)N1] |
| I-266 | 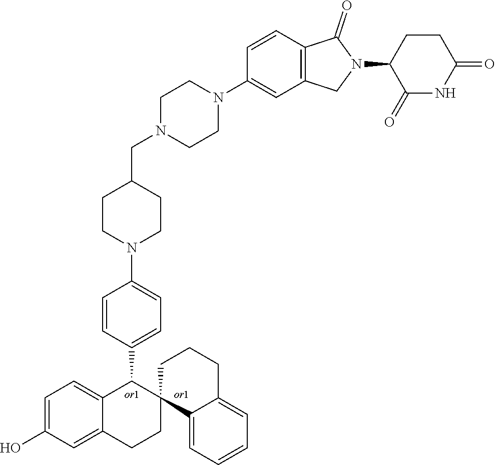 [SMILES: O=C1CC[C@H](N2Cc3cc(N4CCN(CC5CCN(c6ccc([C@H]7c8ccc(O)cc8CC[C@@]78CCCc7ccccc78)cc6)CC5)CC4)ccc3C2=O)C(=O)N1] |
| I-267 | 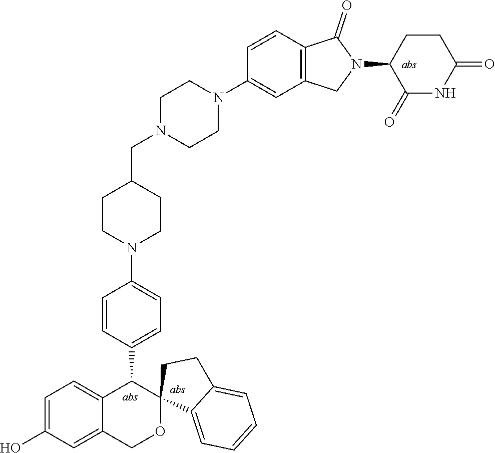 [SMILES: O=C1CC[C@H](N2Cc3cc(N4CCN(CC5CCN(c6ccc([C@H]7c8ccc(O)cc8CO[C@]78CCc7ccccc78)cc6)CC5)CC4)ccc3C2=O)C(=O)N1] |
| I-268 | 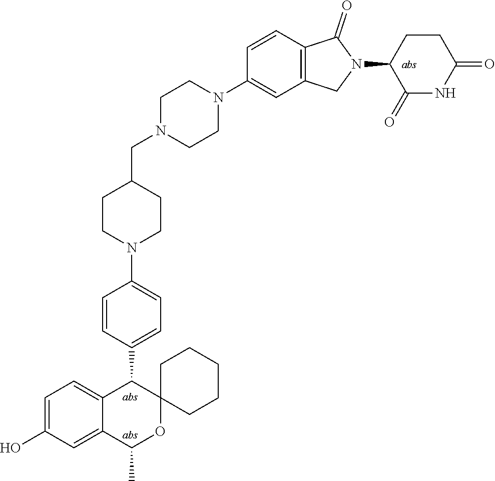 [SMILES: C[C@H]1OC2(CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-269 | 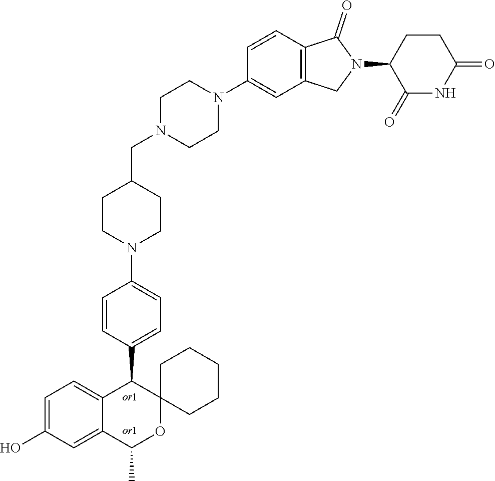 [SMILES: C[C@H]1OC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-270 | 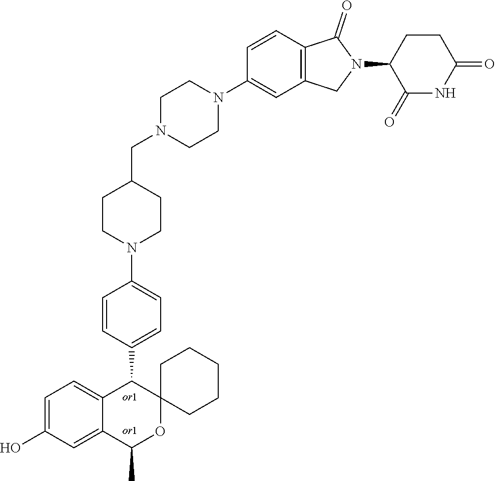 [SMILES: C[C@@H]1OC2(CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-271 | 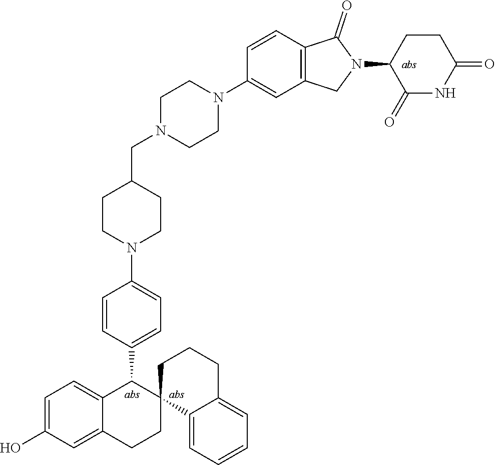 [SMILES: O=C1CC[C@H](N2Cc3cc(N4CCN(CC5CCN(c6ccc([C@H]7c8ccc(O)cc8CC[C@]78CCCc7ccccc78)cc6)CC5)CC4)ccc3C2=O)C(=O)N1] |
| I-272 | 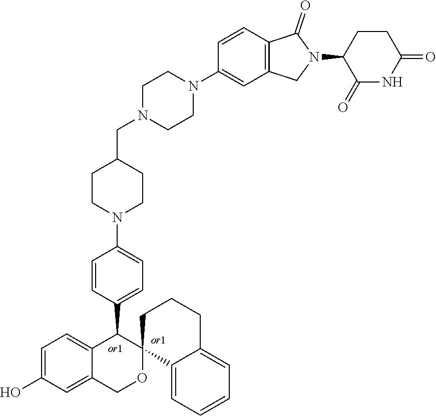 [SMILES: O=C1CC[C@H](N2Cc3cc(N4CCN(CC5CCN(c6ccc([C@@H]7c8ccc(O)cc8CO[C@]78CCCc7ccccc78)cc6)CC5)CC4)ccc3C2=O)C(=O)N1] |
| I-273 | 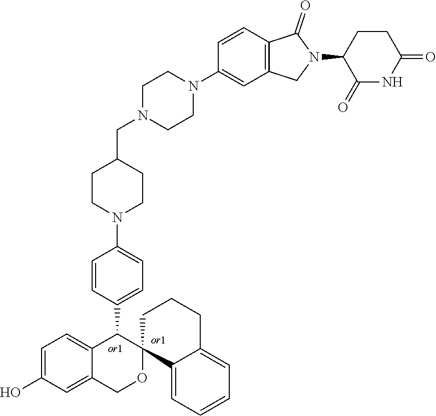 [SMILES: O=C1CC[C@H](N2Cc3cc(N4CCN(CC5CCN(c6ccc([C@H]7c8ccc(O)cc8CO[C@@]78CCCc7ccccc78)cc6)CC5)CC4)ccc3C2=O)C(=O)N1] |
| I-274 | 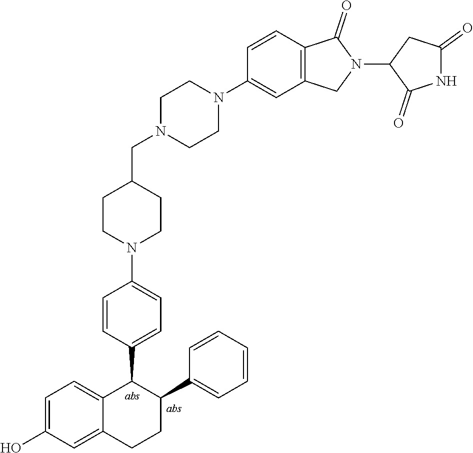 [SMILES: O=C1CC(N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] |
| I-275 | 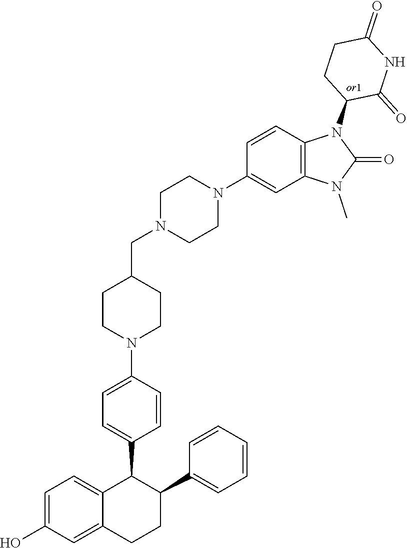 [SMILES: Cn1c(=O)n([C@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-276 | 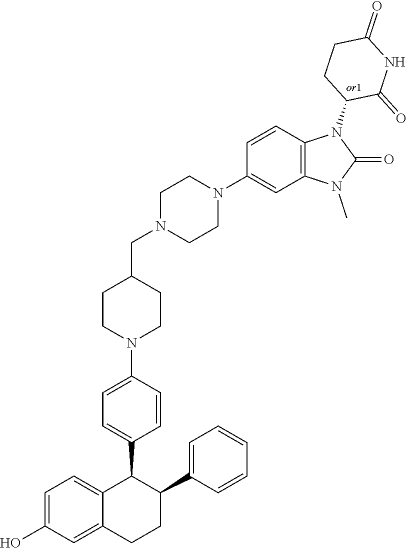 [SMILES: Cn1c(=O)n([C@@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-277 | 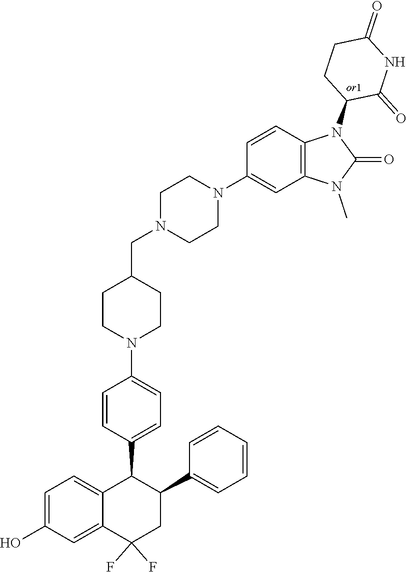 [SMILES: Cn1c(=O)n([C@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] |
| I-278 | 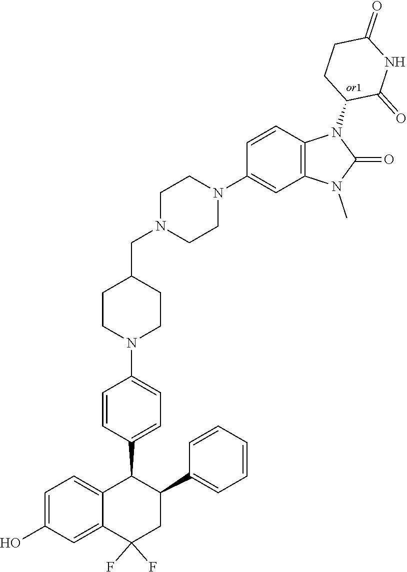 [SMILES: Cn1c(=O)n([C@@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] |
| I-279 | 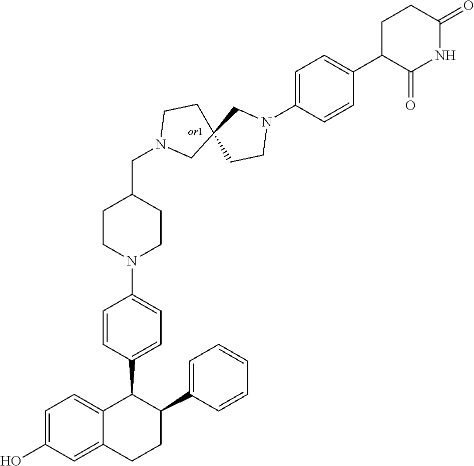 [SMILES: O=C1CCC(c2ccc(N3CC[C@]4(CCN(CC5CCN(c6ccc([C@@H]7c8ccc(O)cc8CC[C@@H]7c7ccccc7)cc6)CC5)C4)C3)cc2)C(=O)N1] |
| I-280 | 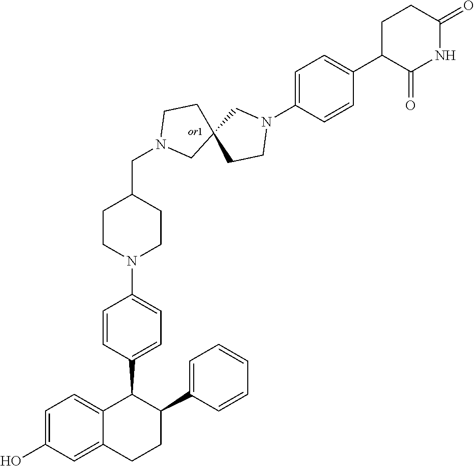 [SMILES: O=C1CCC(c2ccc(N3CC[C@@]4(CCN(CC5CCN(c6ccc([C@@H]7c8ccc(O)cc8CC[C@@H]7c7ccccc7)cc6)CC5)C4)C3)cc2)C(=O)N1] |
| I-281 | 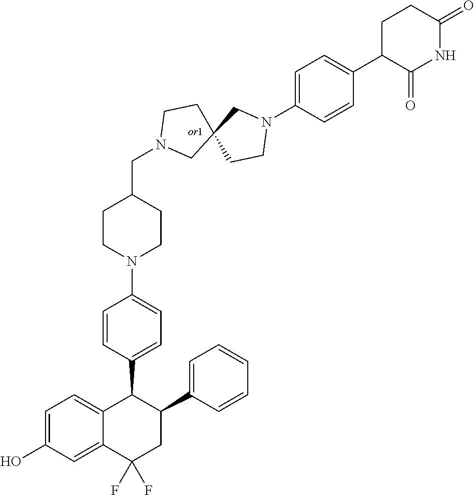 [SMILES: O=C1CCC(c2ccc(N3CC[C@]4(CCN(CC5CCN(c6ccc([C@@H]7c8ccc(O)cc8C(F)(F)C[C@@H]7c7ccccc7)cc6)CC5)C4)C3)cc2)C(=O)N1] |
| I-282 | 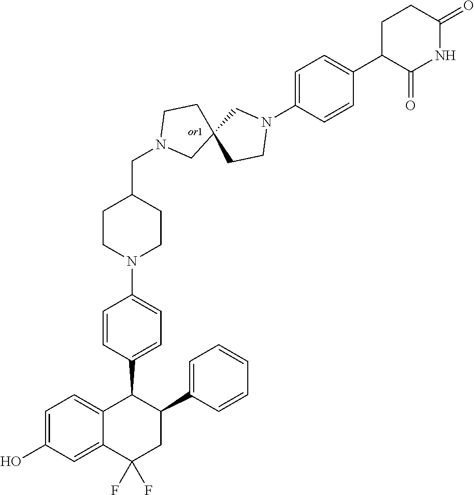 [SMILES: O=C1CCC(c2ccc(N3CC[C@@]4(CCN(CC5CCN(c6ccc([C@@H]7c8ccc(O)cc8C(F)(F)C[C@@H]7c7ccccc7)cc6)CC5)C4)C3)cc2)C(=O)N1] |
| I-283 | 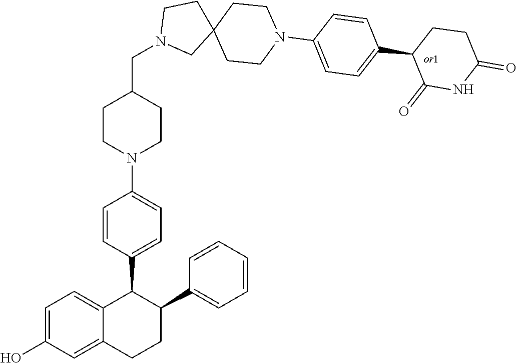 [SMILES: O=C1CC[C@H](c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1] |
| I-284 | 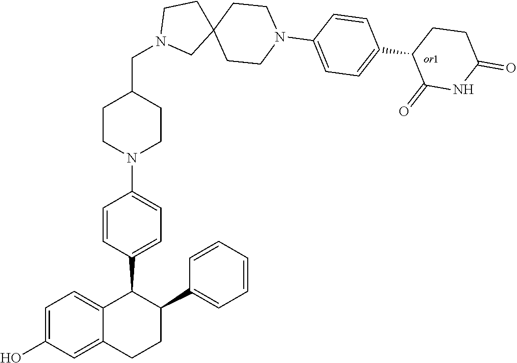 [SMILES: O=C1CC[C@@H](c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1] |
| I-285 | 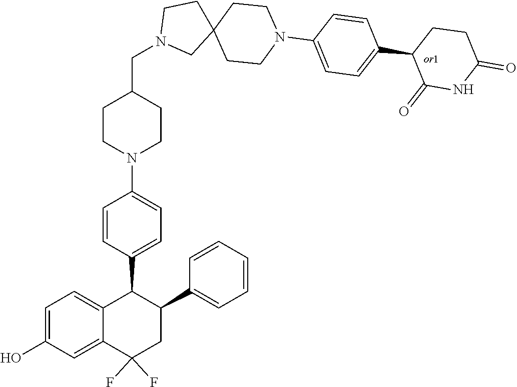 [SMILES: O=C1CC[C@H](c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1] |
| I-286 | 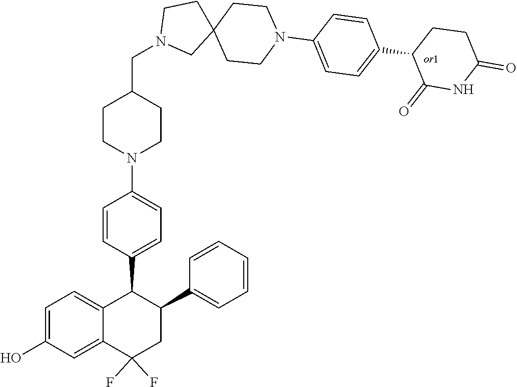 [SMILES: O=C1CC[C@@H](c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1] |
| I-287 | 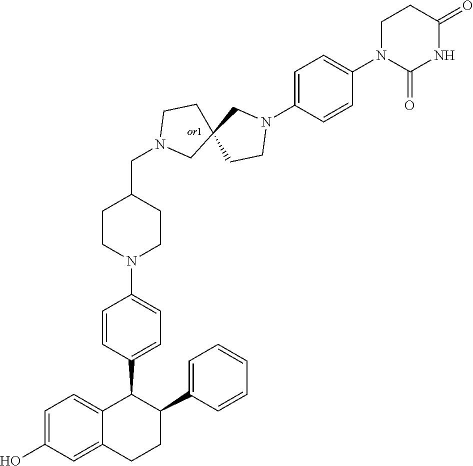 [SMILES: O=C1CCN(c2ccc(N3CC[C@]4(CCN(CC5CCN(c6ccc([C@@H]7c8ccc(O)cc8CC[C@@H]7c7ccccc7)cc6)CC5)C4)C3)cc2)C(=O)N1] |
| I-288 | 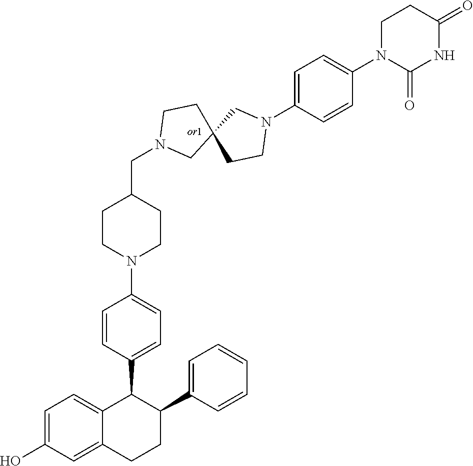 [SMILES: O=C1CCN(c2ccc(N3CC[C@@]4(CCN(CC5CCN(c6ccc([C@@H]7c8ccc(O)cc8CC[C@@H]7c7ccccc7)cc6)CC5)C4)C3)cc2)C(=O)N1] |
| I-289 | 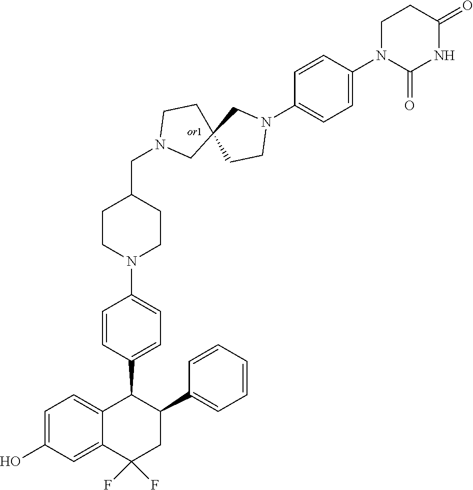 [SMILES: O=C1CCN(c2ccc(N3CC[C@]4(CCN(CC5CCN(c6ccc([C@@H]7c8ccc(O)cc8C(F)(F)C[C@@H]7c7ccccc7)cc6)CC5)C4)C3)cc2)C(=O)N1] |
| I-290 | 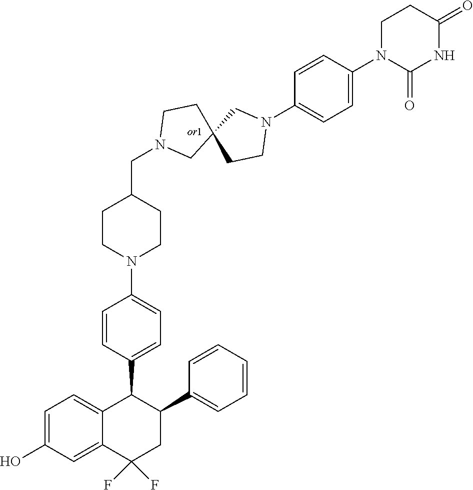 [SMILES: O=C1CCN(c2ccc(N3CC[C@@]4(CCN(CC5CCN(c6ccc([C@@H]7c8ccc(O)cc8C(F)(F)C[C@@H]7c7ccccc7)cc6)CC5)C4)C3)cc2)C(=O)N1] |
| I-291 | 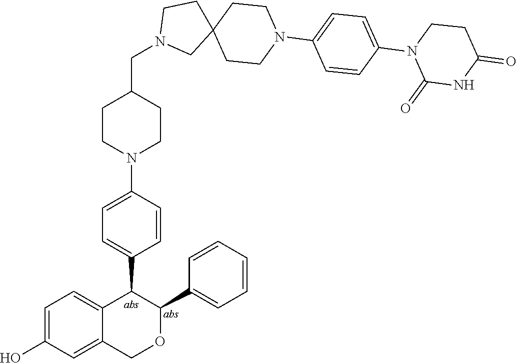 [SMILES: O=C1CCN(c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1] |
| I-292 | 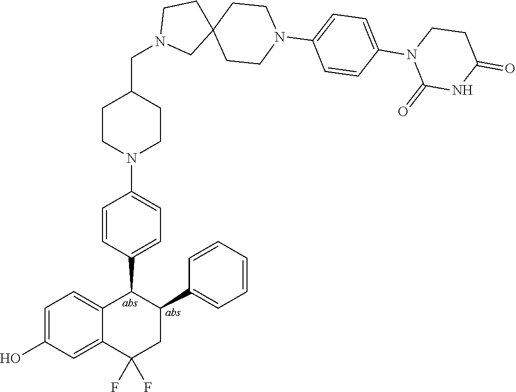 [SMILES: O=C1CCN(c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1] |
| I-293 | 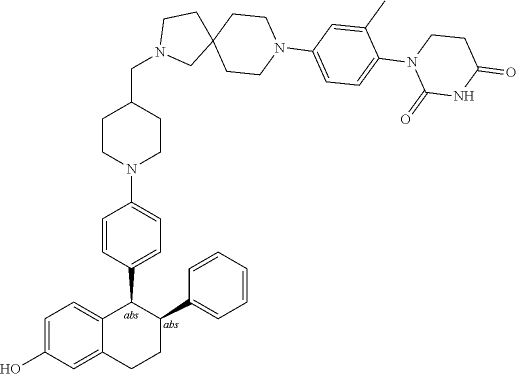 [SMILES: Cc1cc(N2CCC3(CCN(CC4CCN(c5ccc([C@@H]6c7ccc(O)cc7CC[C@@H]6c6ccccc6)cc5)CC4)C3)CC2)ccc1N1CCC(=O)NC1=O] |
| I-294 | 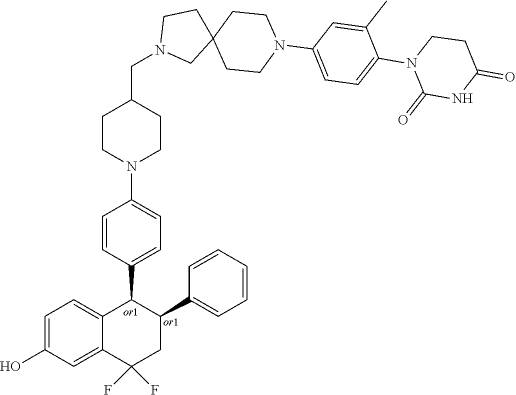 [SMILES: Cc1cc(N2CCC3(CCN(CC4CCN(c5ccc([C@@H]6c7ccc(O)cc7C(F)(F)C[C@@H]6c6ccccc6)cc5)CC4)C3)CC2)ccc1N1CCC(=O)NC1=O] |
| I-295 | 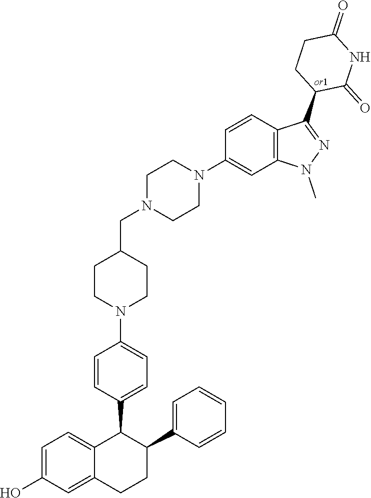 [SMILES: Cn1nc([C@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-296 | 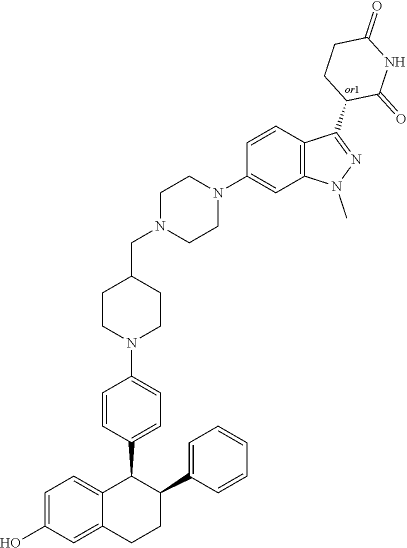 [SMILES: Cn1nc([C@@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-297 | 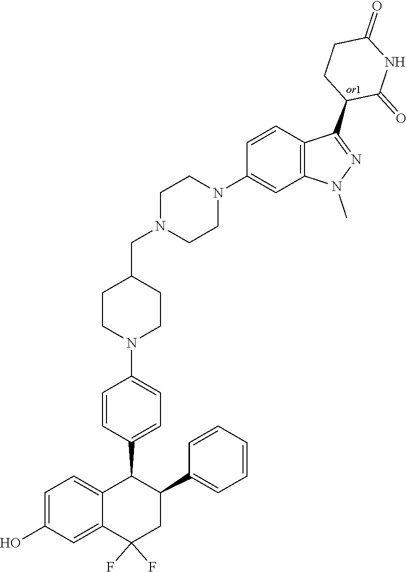 [SMILES: Cn1nc([C@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] |
| I-298 | 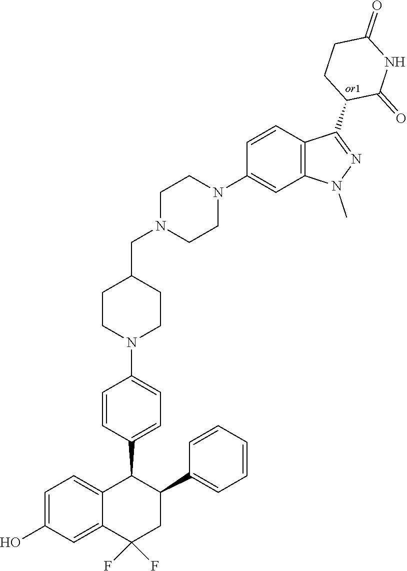 [SMILES: Cn1nc([C@@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] |
| I-299 | 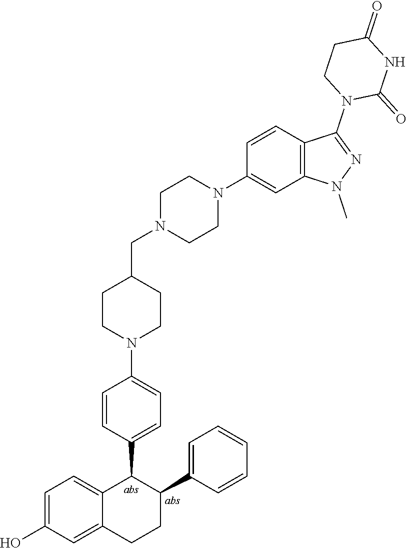 [SMILES: Cn1nc(N2CCC(=O)NC2=O)c2ccc(N3CCN(CC4CCN(c5ccc([C@@H]6c7ccc(O)cc7CC[C@@H]6c6ccccc6)cc5)CC4)CC3)cc21] |
| I-300 | 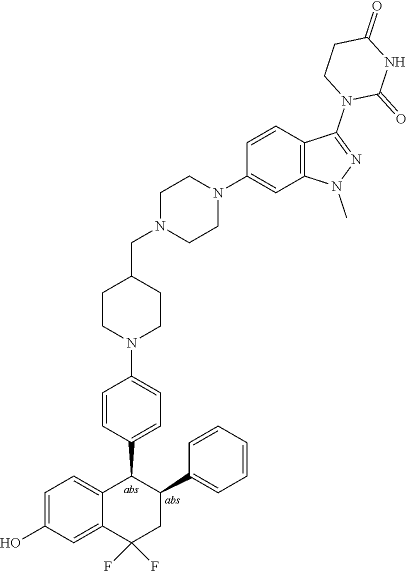 [SMILES: Cn1nc(N2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] |
| I-301 | 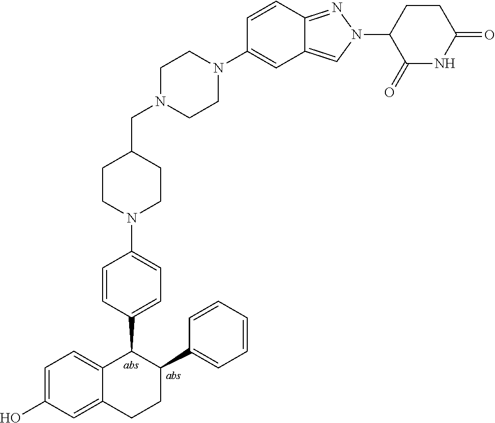 [SMILES: O=C1CCC(n2cc3cc(N4CCN(CC5CCN(c6ccc([C@@H]7c8ccc(O)cc8CC[C@@H]7c7ccccc7)cc6)CC5)CC4)ccc3n2)C(=O)N1] |
| I-302 | 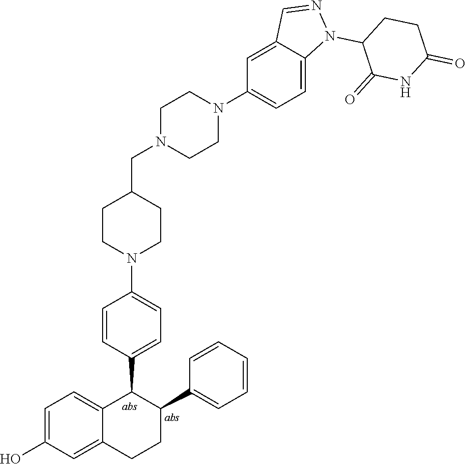 [SMILES: O=C1CCC(n2ncc3cc(N4CCN(CC5CCN(c6ccc([C@@H]7c8ccc(O)cc8CC[C@@H]7c7ccccc7)cc6)CC5)CC4)ccc32)C(=O)N1] |
| I-304 | 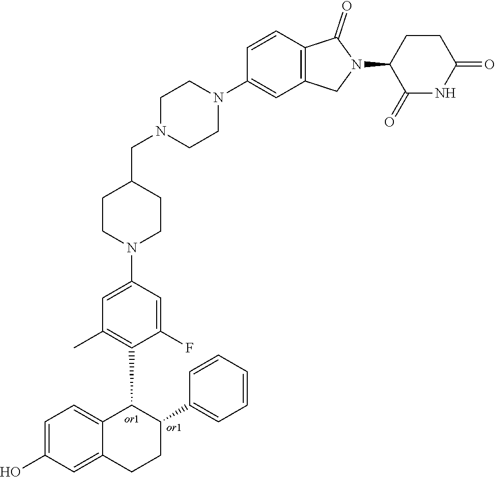 [SMILES: Cc1cc(N2CCC(CN3CCN(c4ccc5c(c4)CN([C@H]4CCC(=O)NC4=O)C5=O)CC3)CC2)cc(F)c1[C@H]1c2ccc(O)cc2CC[C@H]1c1ccccc1] |
| I-305 | 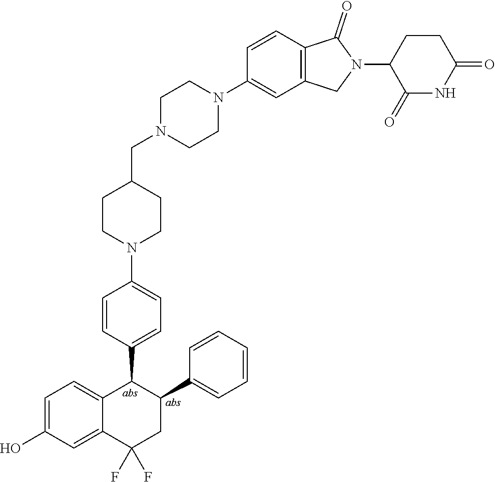 [SMILES: O=C1CCC(N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1] |
| I-306 | 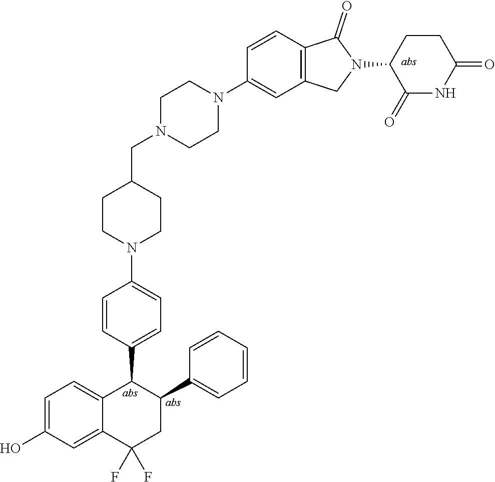 [SMILES: O=C1CC[C@@H](N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1] |
| I-307 | 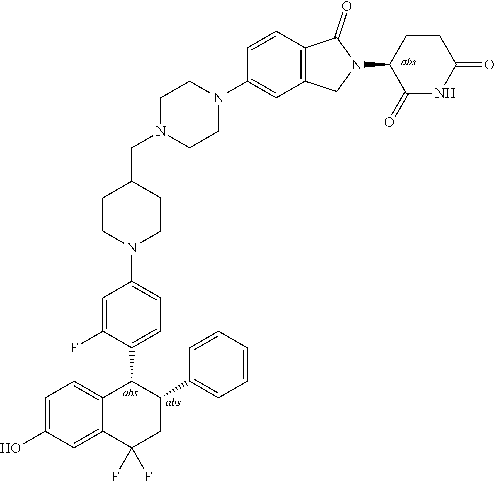 [SMILES: O=C1CC[C@H](N2Cc3cc(N4CCN(CC5CCN(c6ccc([C@H]7c8ccc(O)cc8C(F)(F)C[C@H]7c7ccccc7)c(F)c6)CC5)CC4)ccc3C2=O)C(=O)N1] |
| I-308 | 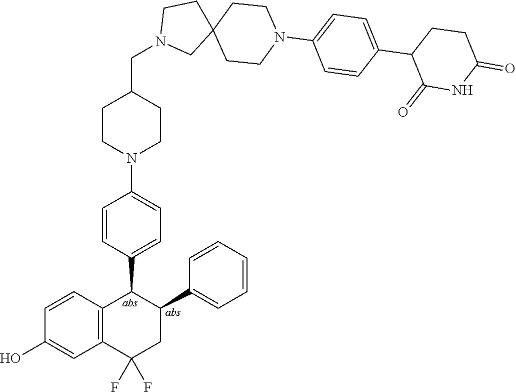 [SMILES: O=C1CCC(c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1] |
| I-309 | 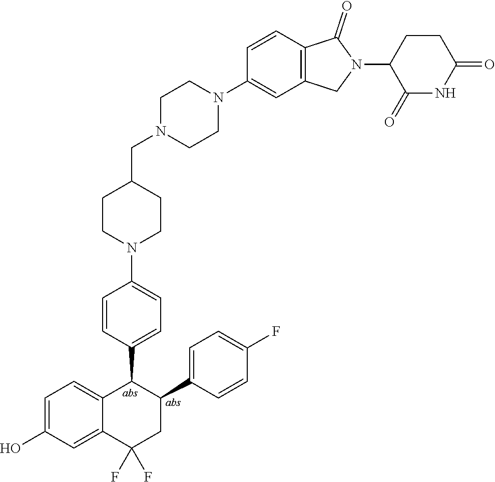 [SMILES: O=C1CCC(N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1] |
| I-310 | 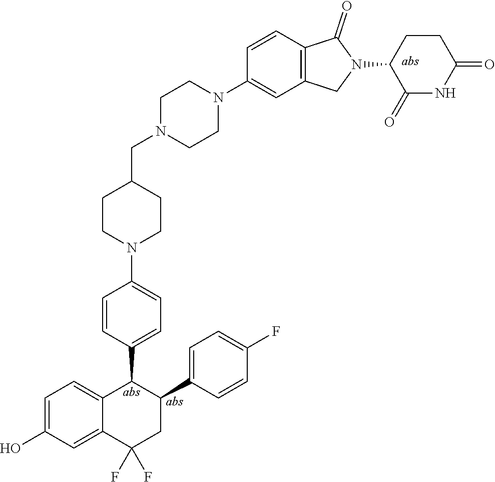 [SMILES: O=C1CC[C@@H](N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1] |
| I-311 | 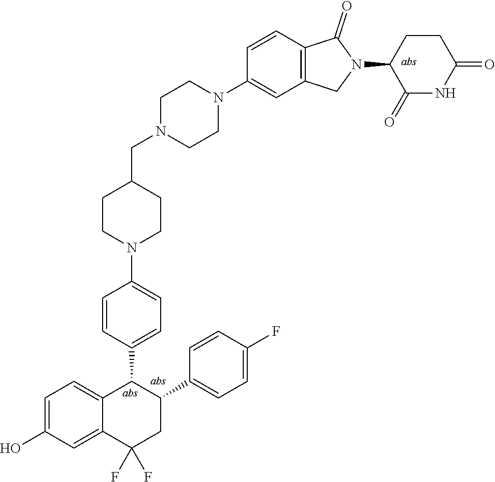 [SMILES: O=C1CC[C@H](N2Cc3cc(N4CCN(CC5CCN(c6ccc([C@H]7c8ccc(O)cc8C(F)(F)C[C@H]7c7ccc(F)cc7)cc6)CC5)CC4)ccc3C2=O)C(=O)N1] |
| I-312 | 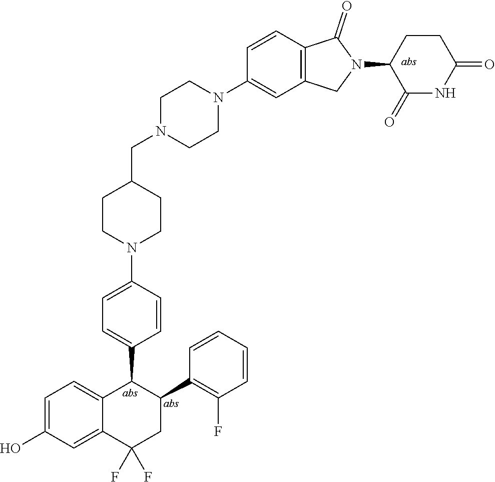 [SMILES: O=C1CC[C@H](N2Cc3cc(N4CCN(CC5CCN(c6ccc([C@@H]7c8ccc(O)cc8C(F)(F)C[C@@H]7c7ccccc7F)cc6)CC5)CC4)ccc3C2=O)C(=O)N1] |
| I-313 | 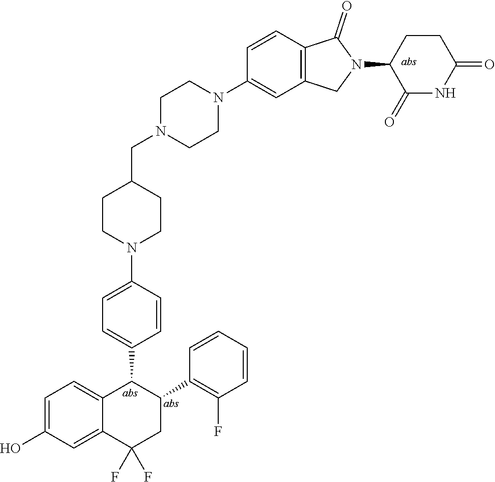 [SMILES: O=C1CC[C@H](N2Cc3cc(N4CCN(CC5CCN(c6ccc([C@H]7c8ccc(O)cc8C(F)(F)C[C@H]7c7ccccc7F)cc6)CC5)CC4)ccc3C2=O)C(=O)N1] |
| I-314 | 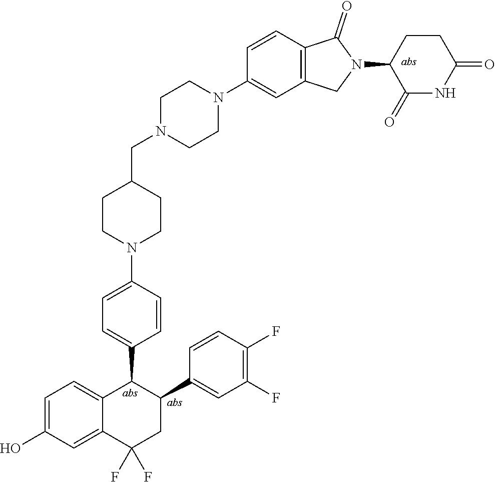 [SMILES: O=C1CC[C@H](N2Cc3cc(N4CCN(CC5CCN(c6ccc([C@@H]7c8ccc(O)cc8C(F)(F)C[C@@H]7c7ccc(F)c(F)c7)cc6)CC5)CC4)ccc3C2=O)C(=O)N1] |
| I-315 | 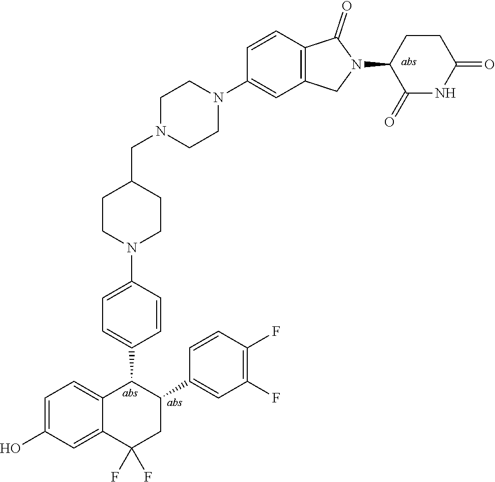 [SMILES: O=C1CC[C@H](N2Cc3cc(N4CCN(CC5CCN(c6ccc([C@H]7c8ccc(O)cc8C(F)(F)C[C@H]7c7ccc(F)c(F)c7)cc6)CC5)CC4)ccc3C2=O)C(=O)N1] |
| I-316 | 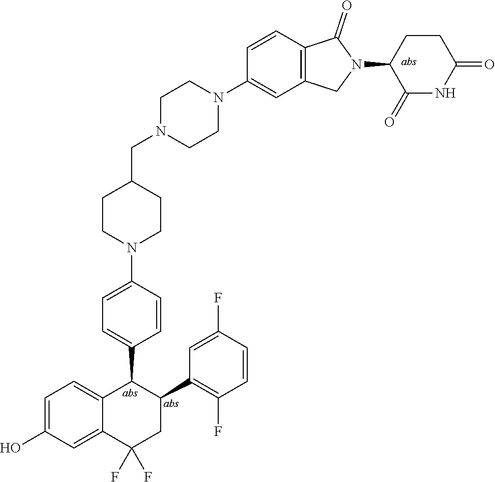 [SMILES: O=C1CC[C@H](N2Cc3cc(N4CCN(CC5CCN(c6ccc([C@@H]7c8ccc(O)cc8C(F)(F)C[C@@H]7c7cc(F)ccc7F)cc6)CC5)CC4)ccc3C2=O)C(=O)N1] |
| I-317 | 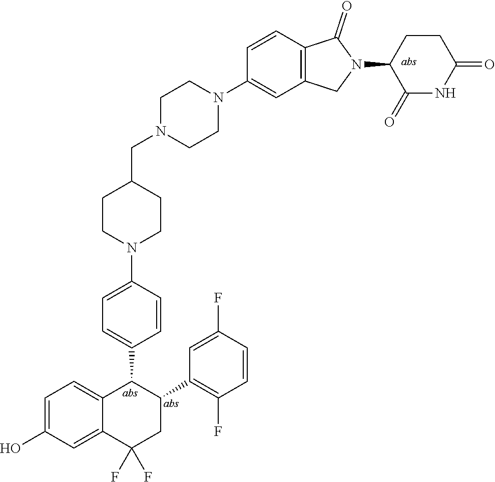 [SMILES: O=C1CC[C@H](N2Cc3cc(N4CCN(CC5CCN(c6ccc([C@H]7c8ccc(O)cc8C(F)(F)C[C@H]7c7cc(F)ccc7F)cc6)CC5)CC4)ccc3C2=O)C(=O)N1] |
| I-318 | 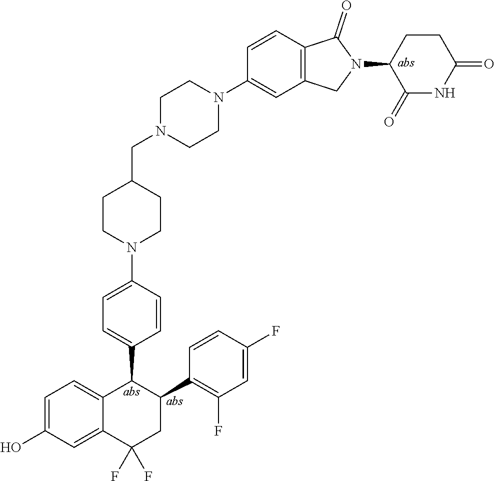 [SMILES: O=C1CC[C@H](N2Cc3cc(N4CCN(CC5CCN(c6ccc([C@@H]7c8ccc(O)cc8C(F)(F)C[C@@H]7c7ccc(F)cc7F)cc6)CC5)CC4)ccc3C2=O)C(=O)N1] |
| I-319 | 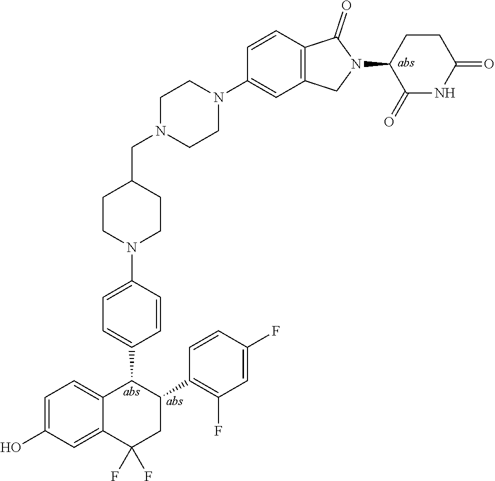 [SMILES: O=C1CC[C@H](N2Cc3cc(N4CCN(CC5CCN(c6ccc([C@H]7c8ccc(O)cc8C(F)(F)C[C@H]7c7ccc(F)cc7F)cc6)CC5)CC4)ccc3C2=O)C(=O)N1] |
| I-320 | 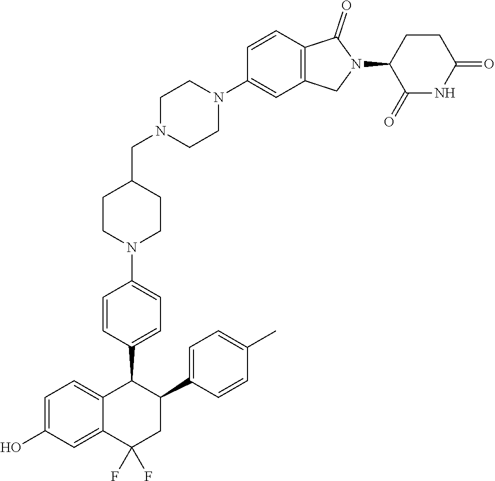 [SMILES: Cc1ccc([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)cc1] |
| I-321 | 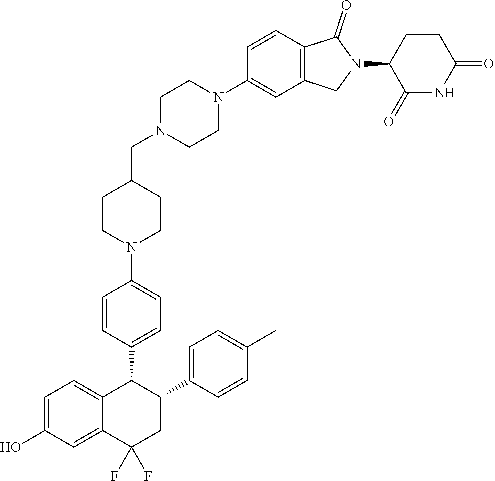 [SMILES: Cc1ccc([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)cc1] |
| I-322 | 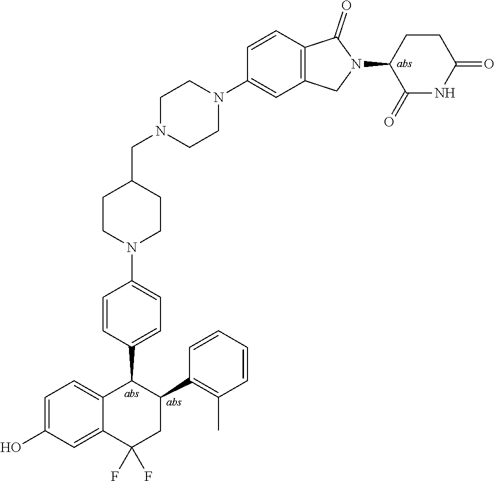 [SMILES: Cc1ccccc1[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-323 | 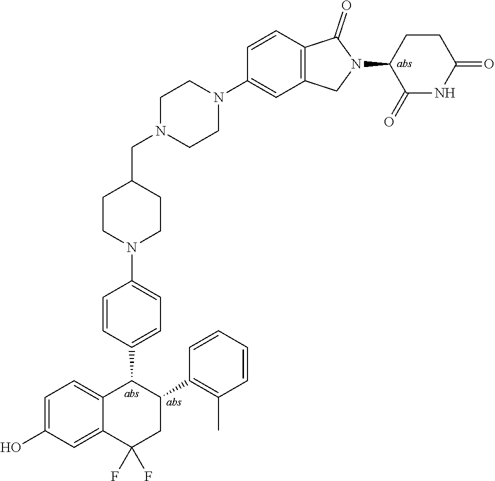 [SMILES: Cc1ccccc1[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-324 | 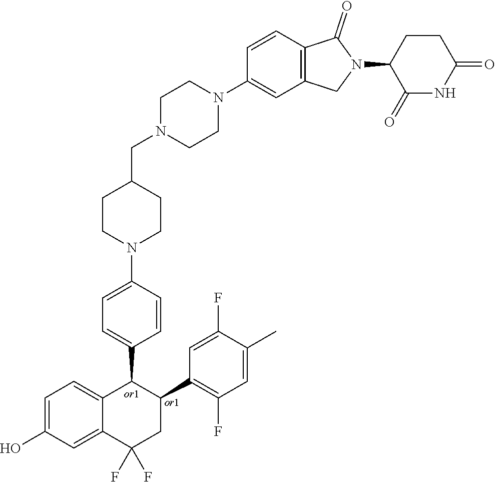 [SMILES: Cc1cc(F)c([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)cc1F] |
| I-325 | 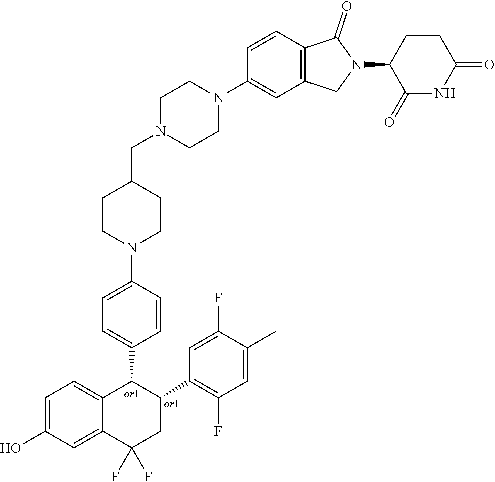 [SMILES: Cc1cc(F)c([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] |
| I-326 | 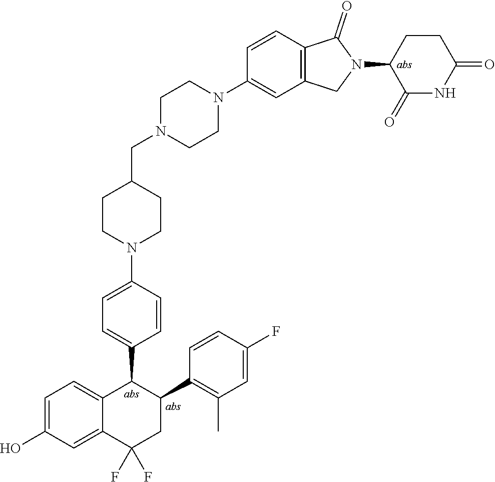 [SMILES: Cc1cc(F)ccc1[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] |
| I-327 | 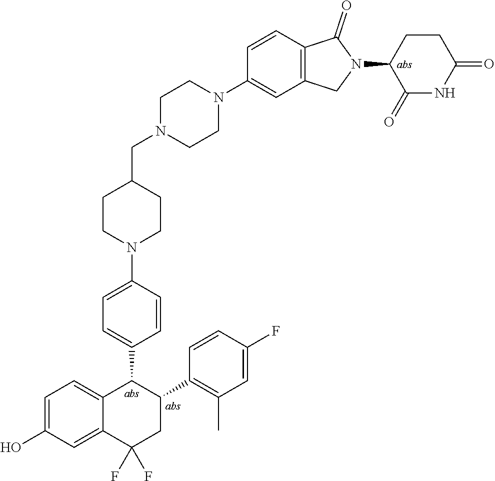 [SMILES: Cc1cc(F)ccc1[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-328 | 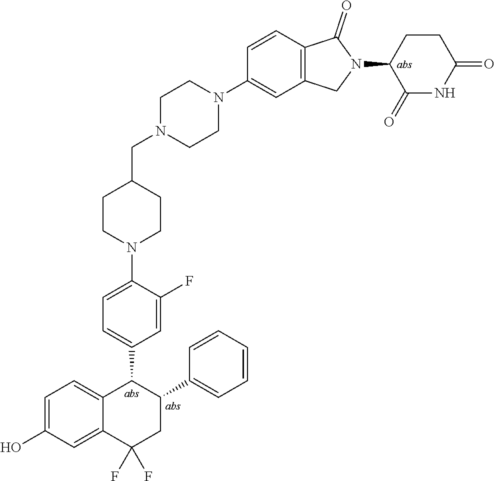 [SMILES: O=C1CC[C@H](N2Cc3cc(N4CCN(CC5CCN(c6ccc([C@H]7c8ccc(O)cc8C(F)(F)C[C@H]7c7ccccc7)cc6F)CC5)CC4)ccc3C2=O)C(=O)N1] |
| I-329 | 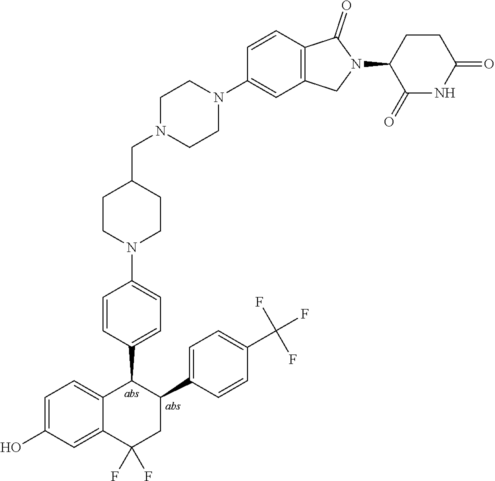 [SMILES: O=C1CC[C@H](N2Cc3cc(N4CCN(CC5CCN(c6ccc([C@@H]7c8ccc(O)cc8C(F)(F)C[C@@H]7c7ccc(C(F)(F)F)cc7)cc6)CC5)CC4)ccc3C2=O)C(=O)N1] |
| I-330 | 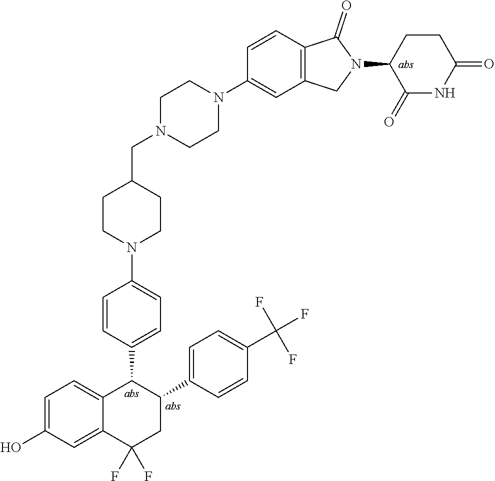 [SMILES: O=C1CC[C@H](N2Cc3cc(N4CCN(CC5CCN(c6ccc([C@H]7c8ccc(O)cc8C(F)(F)C[C@H]7c7ccc(C(F)(F)F)cc7)cc6)CC5)CC4)ccc3C2=O)C(=O)N1] |
| I-331 | 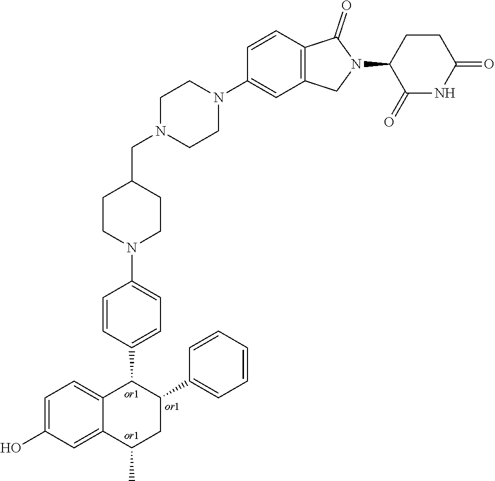 [SMILES: C[C@H]1C[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-332 | 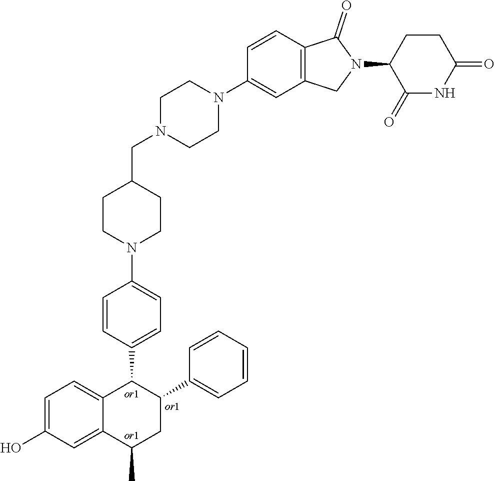 [SMILES: C[C@@H]1C[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-333 | 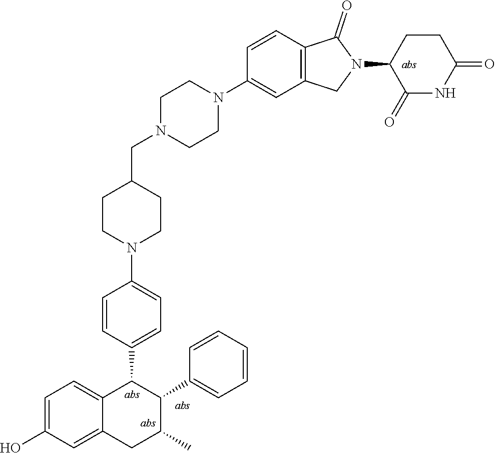 [SMILES: C[C@@H]1Cc2cc(O)ccc2[C@H](c2ccc(N3CCC(CN4CCN(c5ccc6c(c5)CN([C@H]5CCC(=O)NC5=O)C6=O)CC4)CC3)cc2)[C@@H]1c1ccccc1] |
| I-334 | 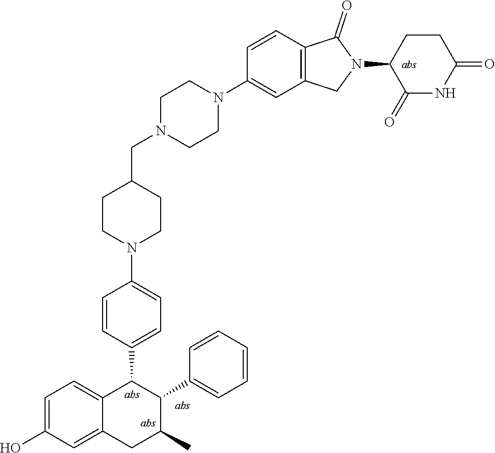 [SMILES: C[C@H]1Cc2cc(O)ccc2[C@H](c2ccc(N3CCC(CN4CCN(c5ccc6c(c5)CN([C@H]5CCC(=O)NC5=O)C6=O)CC4)CC3)cc2)[C@@H]1c1ccccc1] |
| I-335 | 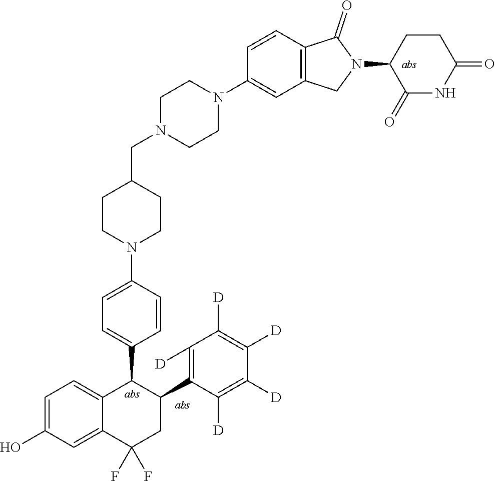 [SMILES: [2H]c1c([2H])c([2H])c([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)c([2H])c1[2H]] |
| I-336 | 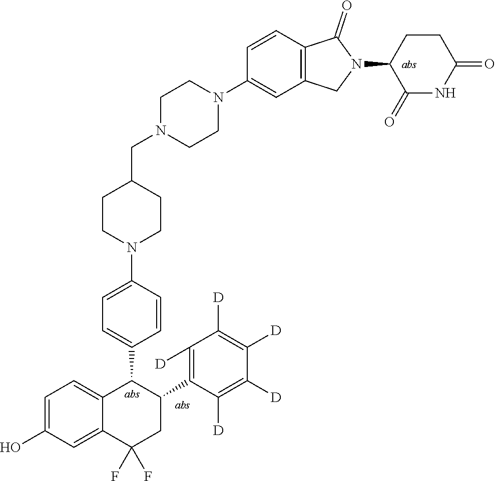 [SMILES: [2H]c1c([2H])c([2H])c([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)c([2H])c1[2H]] |
| I-337 | 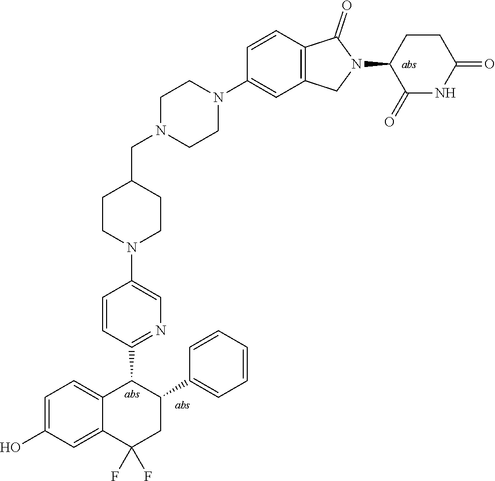 [SMILES: O=C1CC[C@H](N2Cc3cc(N4CCN(CC5CCN(c6ccc([C@H]7c8ccc(O)cc8C(F)(F)C[C@H]7c7ccccc7)nc6)CC5)CC4)ccc3C2=O)C(=O)N1] |
| I-338 | 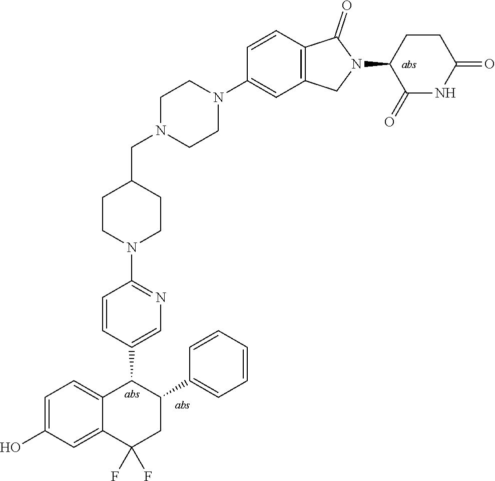 [SMILES: O=C1CC[C@H](N2Cc3cc(N4CCN(CC5CCN(c6ccc([C@H]7c8ccc(O)cc8C(F)(F)C[C@H]7c7ccccc7)cn6)CC5)CC4)ccc3C2=O)C(=O)N1] |
| I-339 | 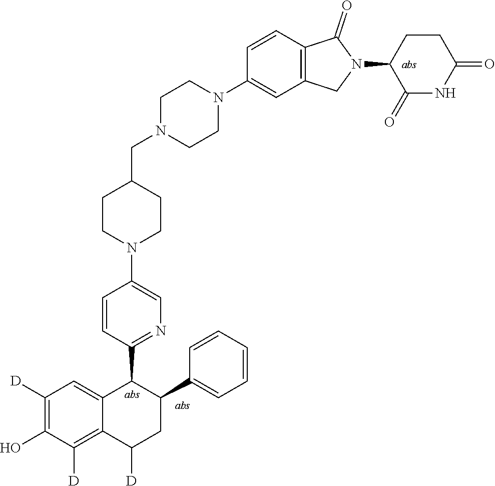 [SMILES: [2H]c1cc2c(c([2H])c1O)C([2H])C[C@H](c1ccccc1)[C@@H]2c1ccc(N2CCC(CN3CCN(c4ccc5c(c4)CN([C@H]4CCC(=O)NC4=O)C5=O)CC3)CC2)cn1] |
| I-340 | 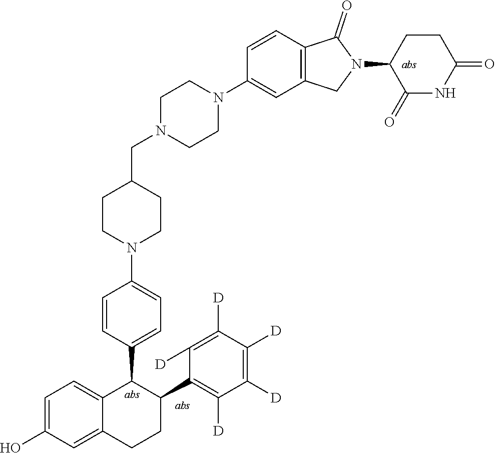 [SMILES: [2H]c1c([2H])c([2H])c([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)c([2H])c1[2H]] |
| I-341 | 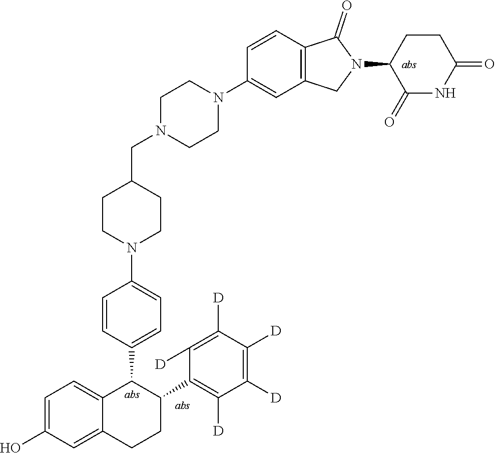 [SMILES: [2H]c1c([2H])c([2H])c([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)c([2H])c1[2H]] |
| I-342 | 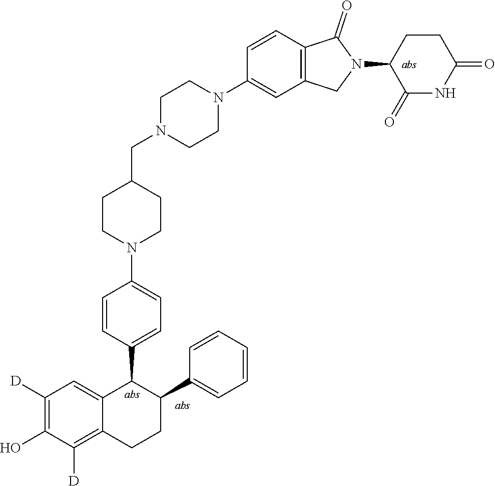 [SMILES: [2H]c1cc2c(c([2H])c1O)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1] |
| I-344 | 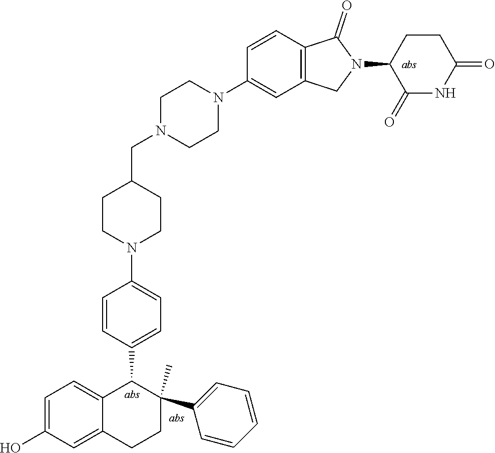 [SMILES: C[C@]1(c2ccccc2)CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-345 | 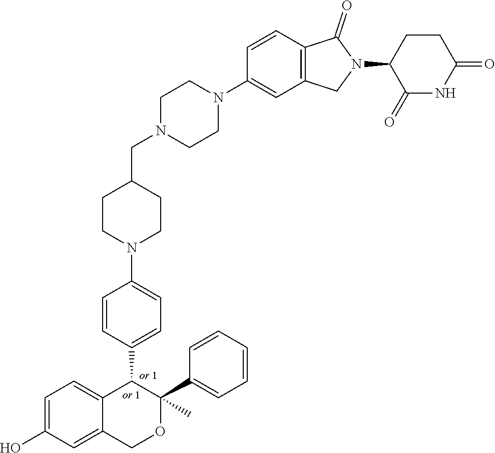 [SMILES: C[C@]1(c2ccccc2)OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-346 | 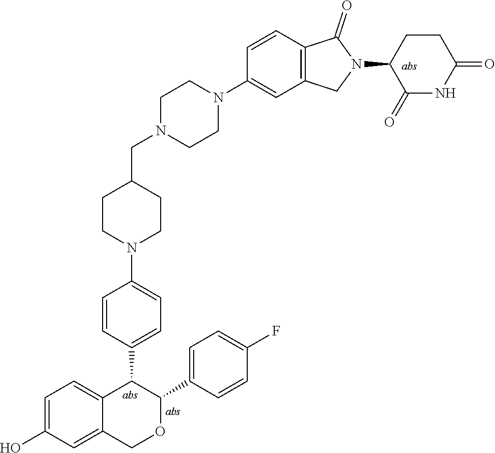 [SMILES: O=C1CC[C@H](N2Cc3cc(N4CCN(CC5CCN(c6ccc([C@H]7c8ccc(O)cc8CO[C@H]7c7ccc(F)cc7)cc6)CC5)CC4)ccc3C2=O)C(=O)N1] |
| I-347 | 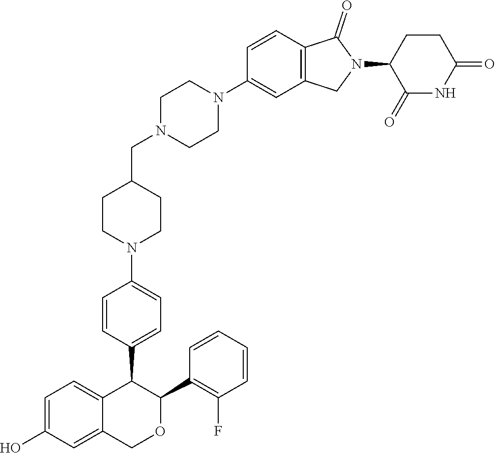 [SMILES: O=C1CC[C@H](N2Cc3cc(N4CCN(CC5CCN(c6ccc([C@@H]7c8ccc(O)cc8CO[C@@H]7c7ccccc7F)cc6)CC5)CC4)ccc3C2=O)C(=O)N1] |
| I-348 | 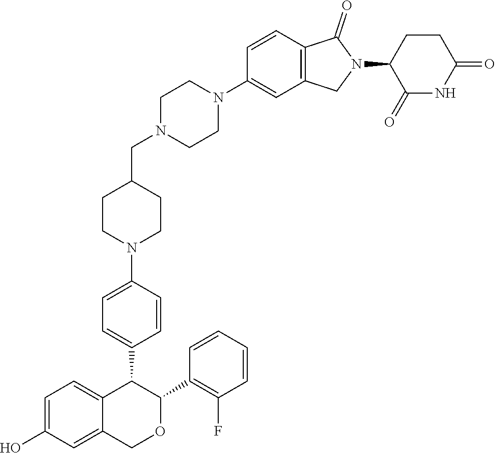 [SMILES: O=C1CC[C@H](N2Cc3cc(N4CCN(CC5CCN(c6ccc([C@H]7c8ccc(O)cc8CO[C@H]7c7ccccc7F)cc6)CC5)CC4)ccc3C2=O)C(=O)N1] |
| I-349 | 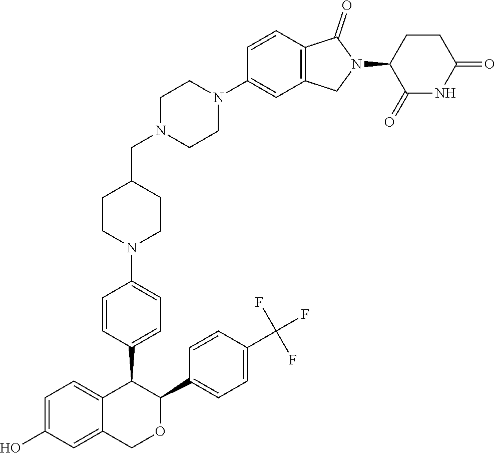 [SMILES: O=C1CC[C@H](N2Cc3cc(N4CCN(CC5CCN(c6ccc([C@@H]7c8ccc(O)cc8CO[C@@H]7c7ccc(C(F)(F)F)cc7)cc6)CC5)CC4)ccc3C2=O)C(=O)N1] |
| I-350 | 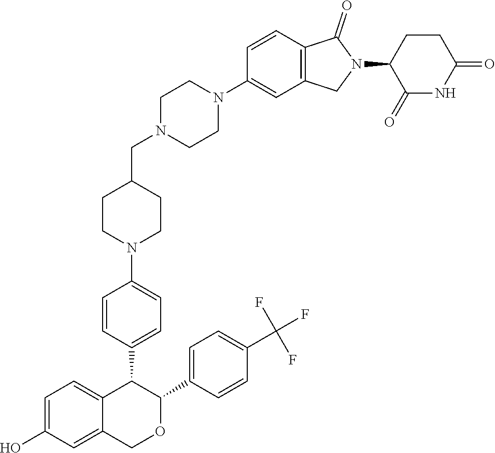 [SMILES: O=C1CC[C@H](N2Cc3cc(N4CCN(CC5CCN(c6ccc([C@H]7c8ccc(O)cc8CO[C@H]7c7ccc(C(F)(F)F)cc7)cc6)CC5)CC4)ccc3C2=O)C(=O)N1] |
| I-351 | 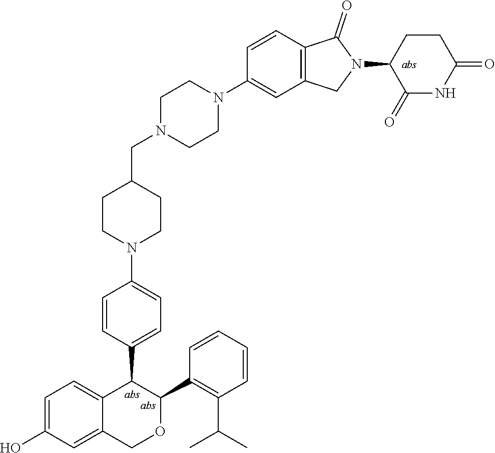 [SMILES: CC(C)c1ccccc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-352 | 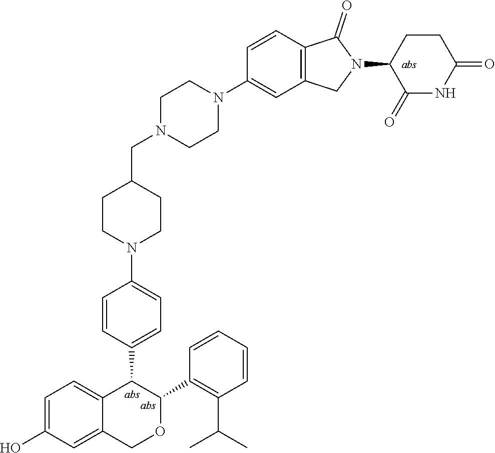 [SMILES: CC(C)c1ccccc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-353 | 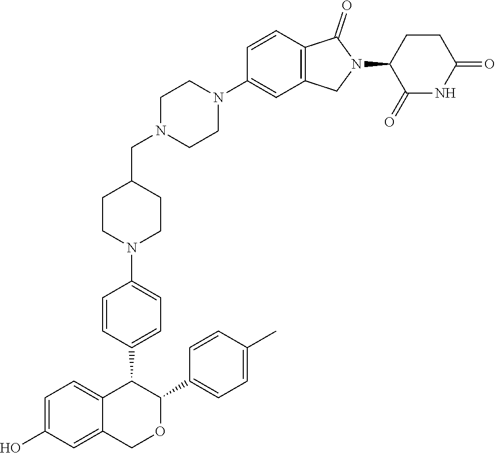 [SMILES: Cc1ccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1] |
| I-354 | 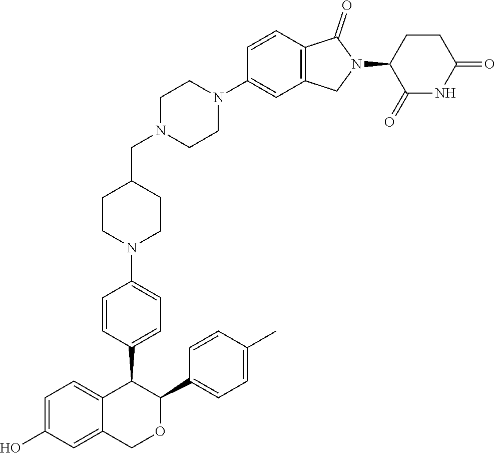 [SMILES: Cc1ccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1] |
| I-355 | 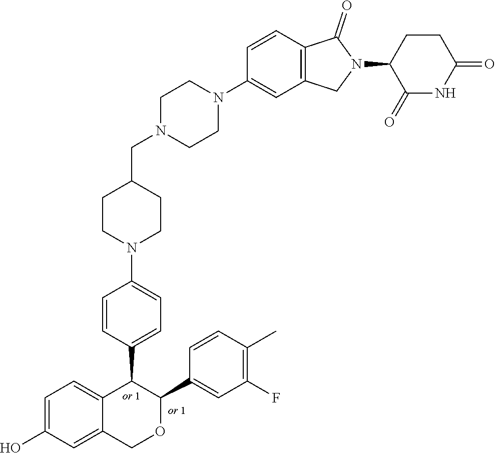 [SMILES: Cc1ccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F] |
| I-356 | 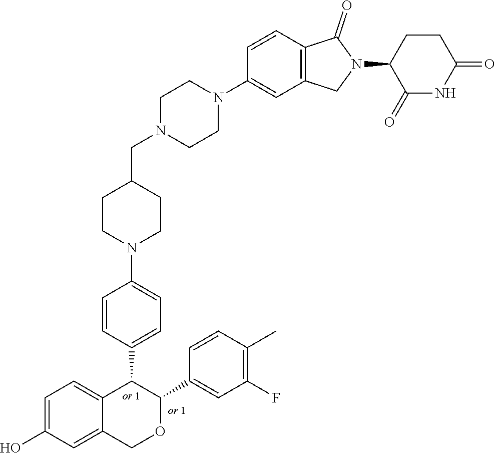 [SMILES: Cc1ccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] |
| I-357 | 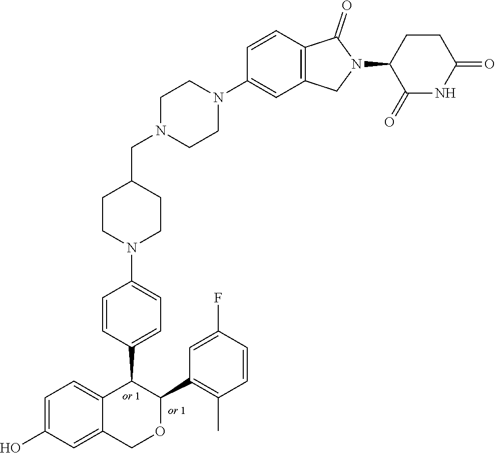 [SMILES: Cc1ccc(F)cc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-358 | 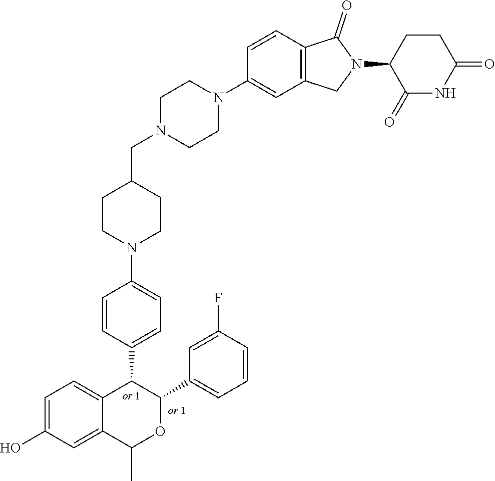 [SMILES: CC1O[C@@H](c2cccc(F)c2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-359 | 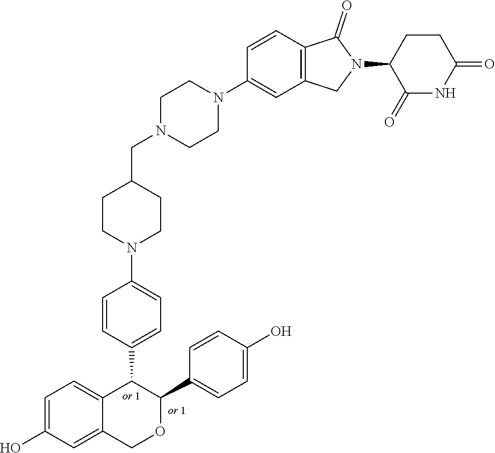 [SMILES: O=C1CC[C@H](N2Cc3cc(N4CCN(CC5CCN(c6ccc([C@H]7c8ccc(O)cc8CO[C@@H]7c7ccc(O)cc7)cc6)CC5)CC4)ccc3C2=O)C(=O)N1] |
| I-360 | 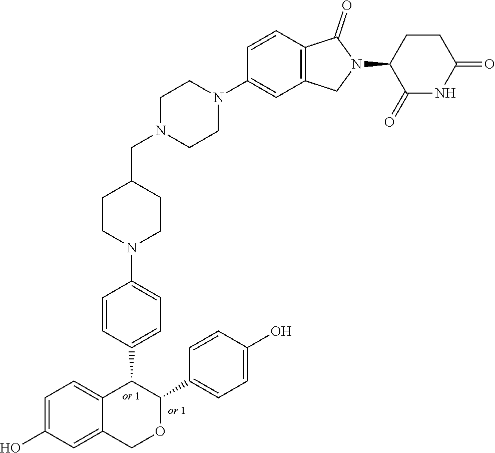 [SMILES: O=C1CC[C@H](N2Cc3cc(N4CCN(CC5CCN(c6ccc([C@H]7c8ccc(O)cc8CO[C@H]7c7ccc(O)cc7)cc6)CC5)CC4)ccc3C2=O)C(=O)N1] |
| I-361 | 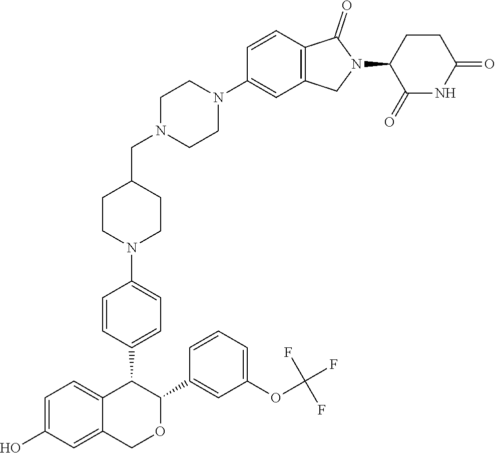 [SMILES: O=C1CC[C@H](N2Cc3cc(N4CCN(CC5CCN(c6ccc([C@H]7c8ccc(O)cc8CO[C@H]7c7cccc(OC(F)(F)F)c7)cc6)CC5)CC4)ccc3C2=O)C(=O)N1] |
| I-362 | 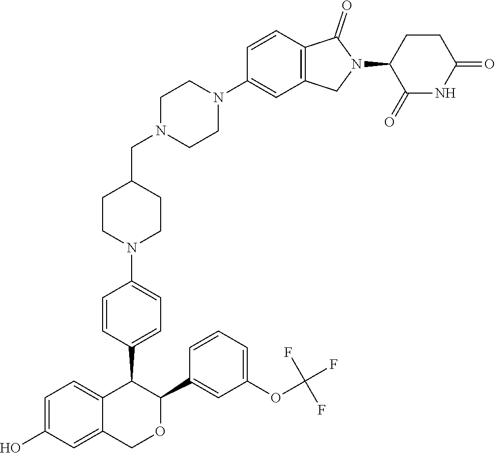 [SMILES: O=C1CC[C@H](N2Cc3cc(N4CCN(CC5CCN(c6ccc([C@@H]7c8ccc(O)cc8CO[C@@H]7c7cccc(OC(F)(F)F)c7)cc6)CC5)CC4)ccc3C2=O)C(=O)N1] |
| I-363 | 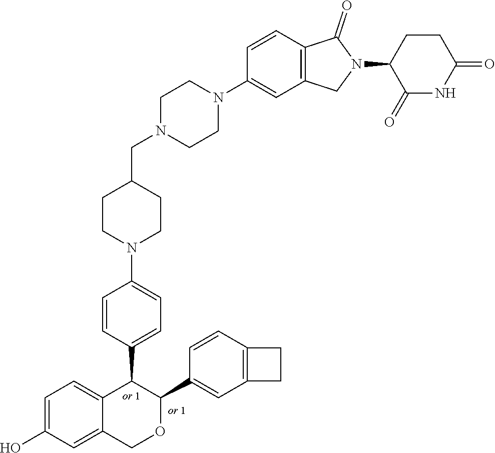 [SMILES: O=C1CC[C@H](N2Cc3cc(N4CCN(CC5CCN(c6ccc([C@@H]7c8ccc(O)cc8CO[C@@H]7c7ccc8c(c7)CC8)cc6)CC5)CC4)ccc3C2=O)C(=O)N1] |
| I-364 | 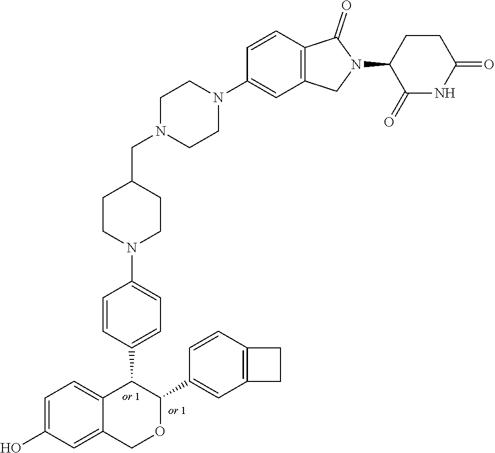 [SMILES: O=C1CC[C@H](N2Cc3cc(N4CCN(CC5CCN(c6ccc([C@H]7c8ccc(O)cc8CO[C@H]7c7ccc8c(c7)CC8)cc6)CC5)CC4)ccc3C2=O)C(=O)N1] |
| I-365 | 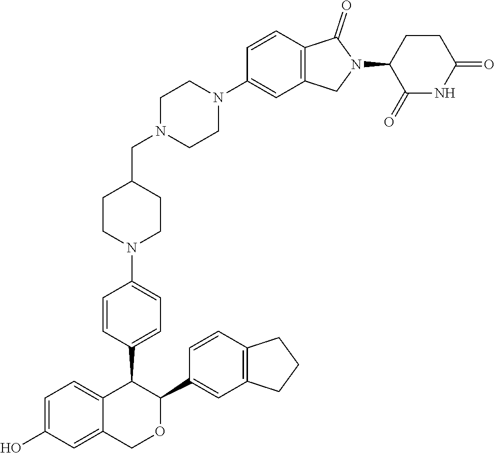 [SMILES: O=C1CC[C@H](N2Cc3cc(N4CCN(CC5CCN(c6ccc([C@@H]7c8ccc(O)cc8CO[C@@H]7c7ccc8c(c7)CCC8)cc6)CC5)CC4)ccc3C2=O)C(=O)N1] |
| I-366 | 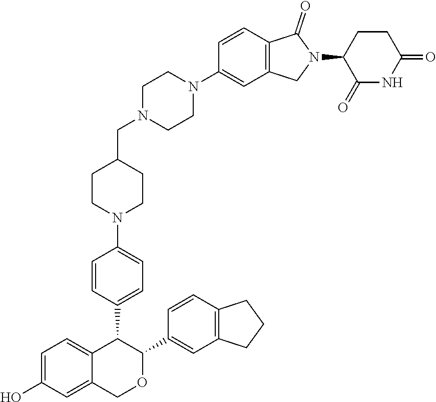 [SMILES: O=C1CC[C@H](N2Cc3cc(N4CCN(CC5CCN(c6ccc([C@H]7c8ccc(O)cc8CO[C@H]7c7ccc8c(c7)CCC8)cc6)CC5)CC4)ccc3C2=O)C(=O)N1] |
| I-367 | 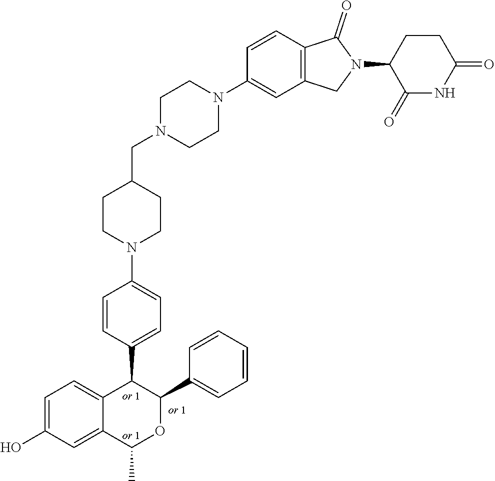 [SMILES: C[C@H]1O[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-368 | 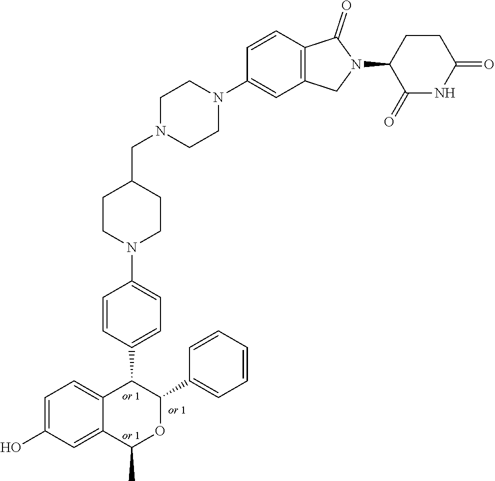 [SMILES: C[C@@H]1O[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21] |
| I-369 | 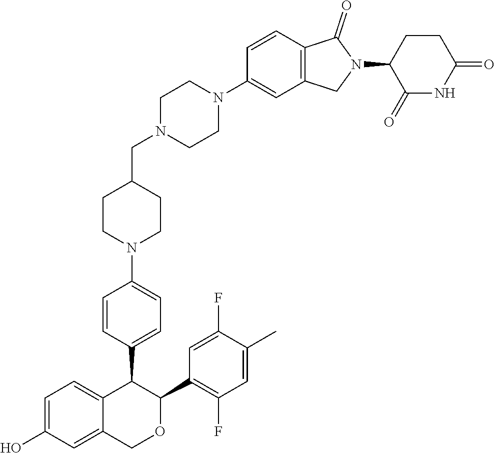 [SMILES: Cc1cc(F)c([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F] |
| I-370 | 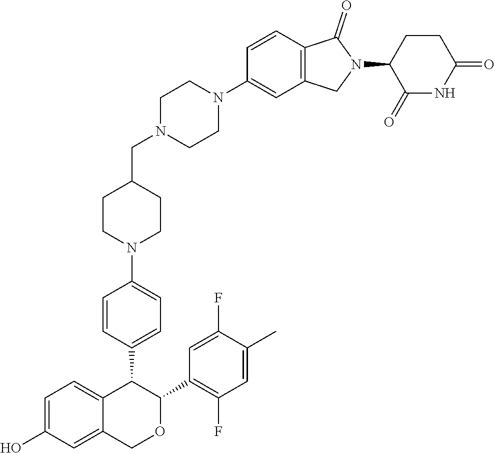 [SMILES: Cc1cc(F)c([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] |
| I-371 | 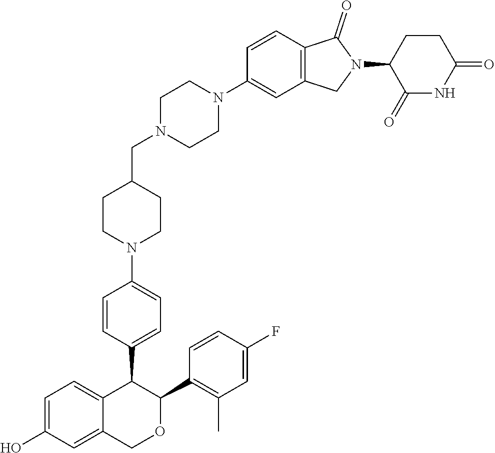 [SMILES: Cc1cc(F)ccc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-372 | 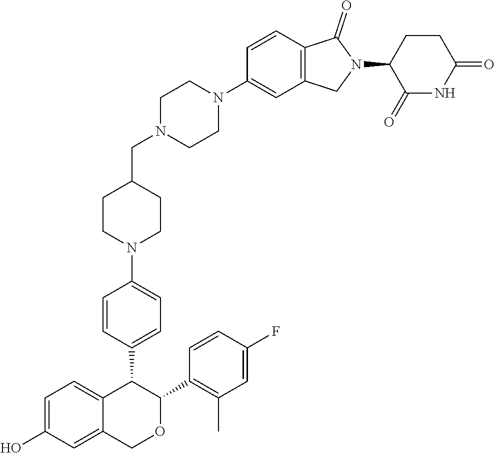 [SMILES: Cc1cc(F)ccc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-373 | 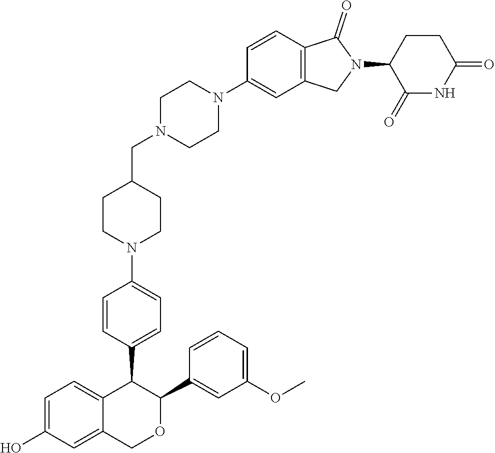 [SMILES: COc1cccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)c1] |
| I-374 | 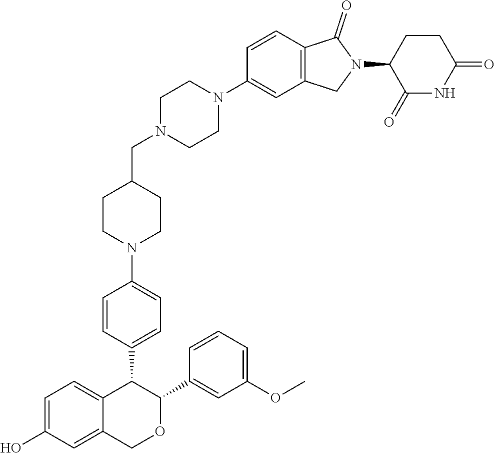 [SMILES: COc1cccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)c1] |
| I-375 | 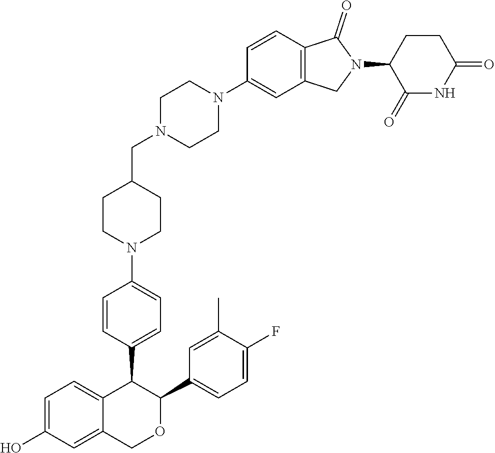 [SMILES: Cc1cc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)ccc1F] |
| I-376 | 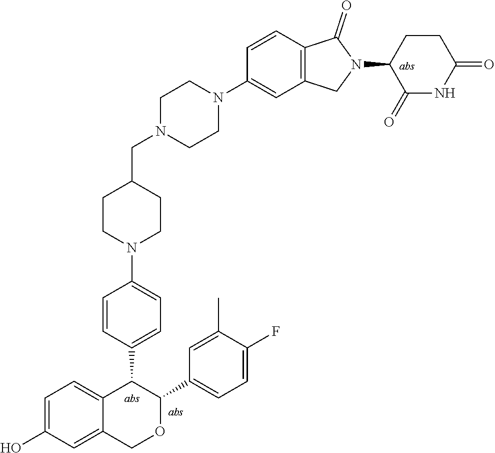 [SMILES: Cc1cc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)ccc1F] |
| I-377 | 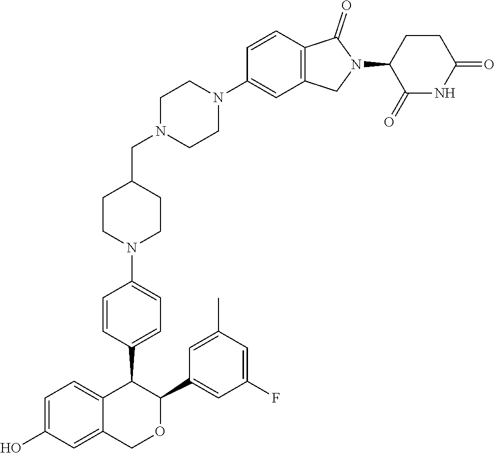 [SMILES: Cc1cc(F)cc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)c1] |
| I-378 | 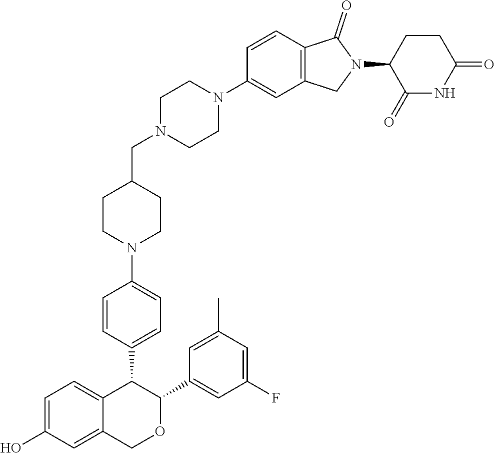 [SMILES: Cc1cc(F)cc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)c1] |
| I-379 | 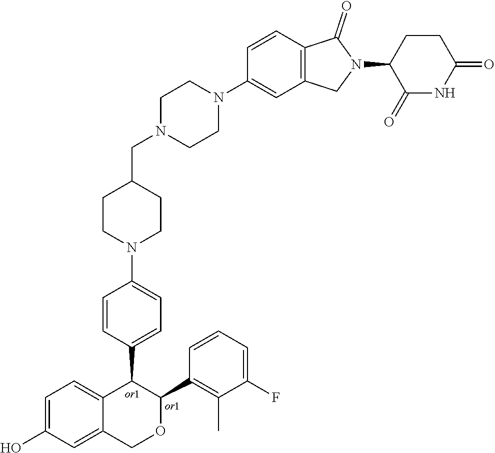 [SMILES: Cc1c(F)cccc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-380 | 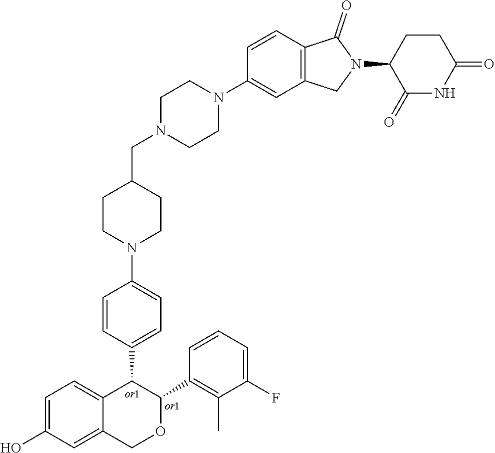 [SMILES: Cc1c(F)cccc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| I-381 | 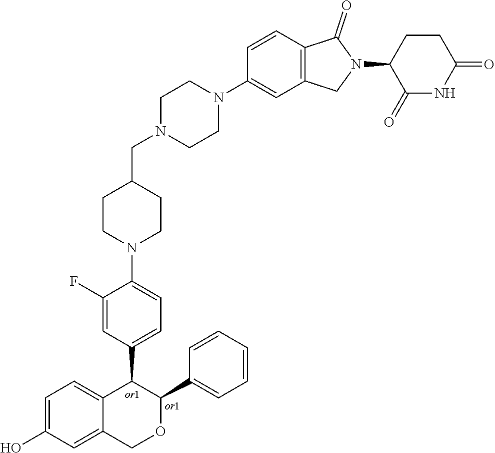 [SMILES: O=C1CC[C@H](N2Cc3cc(N4CCN(CC5CCN(c6ccc([C@@H]7c8ccc(O)cc8CO[C@@H]7c7ccccc7)cc6F)CC5)CC4)ccc3C2=O)C(=O)N1] |
| I-382 | 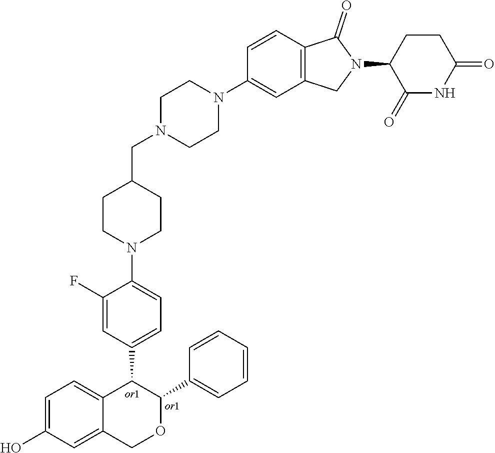 [SMILES: O=C1CC[C@H](N2Cc3cc(N4CCN(CC5CCN(c6ccc([C@H]7c8ccc(O)cc8CO[C@H]7c7ccccc7)cc6F)CC5)CC4)ccc3C2=O)C(=O)N1] |
| I-383 | 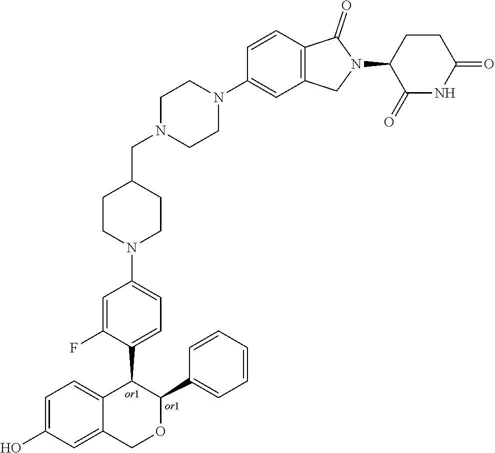 [SMILES: O=C1CC[C@H](N2Cc3cc(N4CCN(CC5CCN(c6ccc([C@@H]7c8ccc(O)cc8CO[C@@H]7c7ccccc7)c(F)c6)CC5)CC4)ccc3C2=O)C(=O)N1] |
| I-384 | 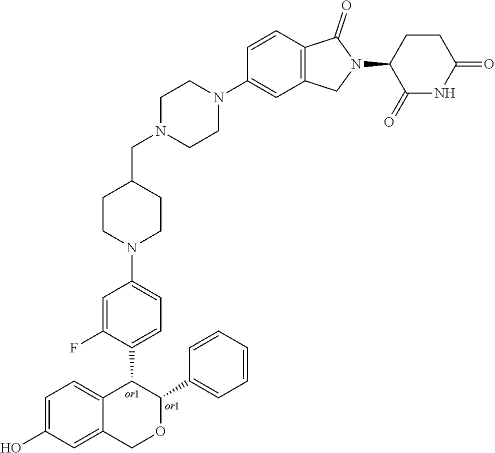 [SMILES: O=C1CC[C@H](N2Cc3cc(N4CCN(CC5CCN(c6ccc([C@H]7c8ccc(O)cc8CO[C@H]7c7ccccc7)c(F)c6)CC5)CC4)ccc3C2=O)C(=O)N1] |
| I-385 | 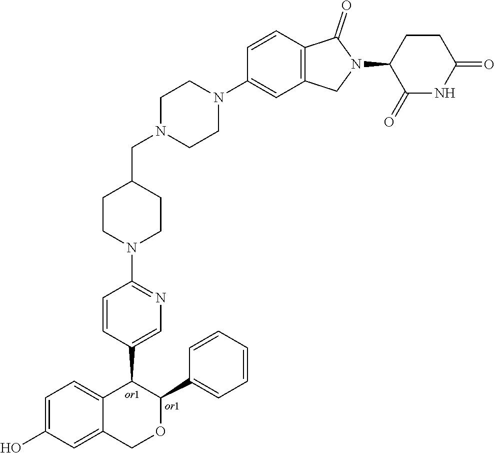 [SMILES: O=C1CC[C@H](N2Cc3cc(N4CCN(CC5CCN(c6ccc([C@@H]7c8ccc(O)cc8CO[C@@H]7c7ccccc7)cn6)CC5)CC4)ccc3C2=O)C(=O)N1] |
| I-386 | 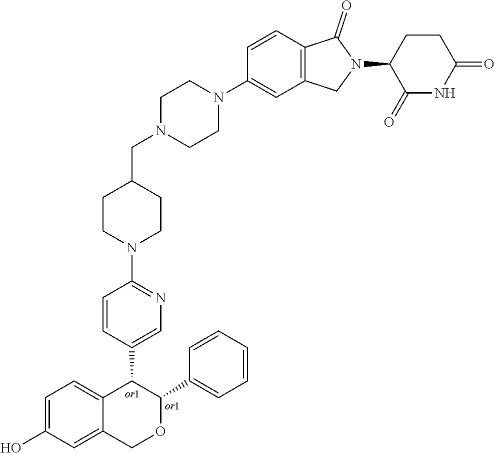 [SMILES: O=C1CC[C@H](N2Cc3cc(N4CCN(CC5CCN(c6ccc([C@H]7c8ccc(O)cc8CO[C@H]7c7ccccc7)cn6)CC5)CC4)ccc3C2=O)C(=O)N1] |
| I-387 | 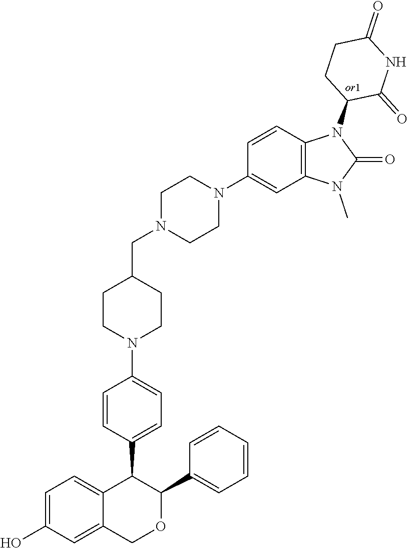 [SMILES: Cn1c(=O)n([C@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21] |
| I-388 | 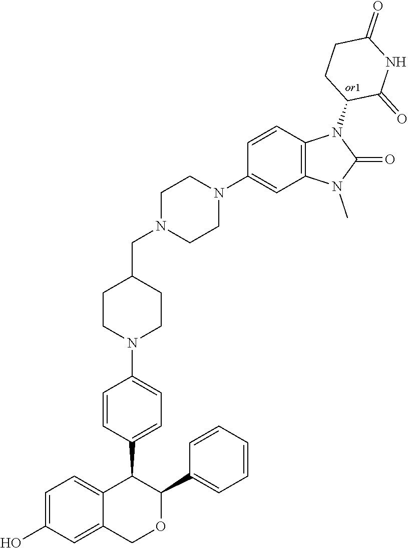 [SMILES: Cn1c(=O)n([C@@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21] |
| I-389 | 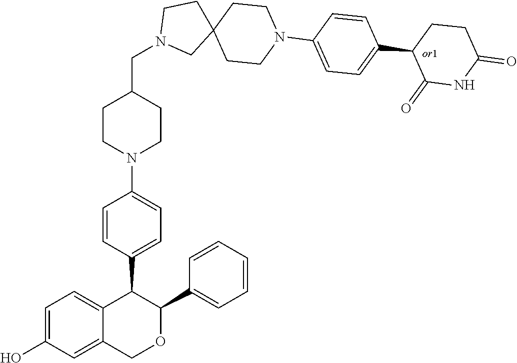 [SMILES: O=C1CC[C@H](c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1] |
| I-390 | 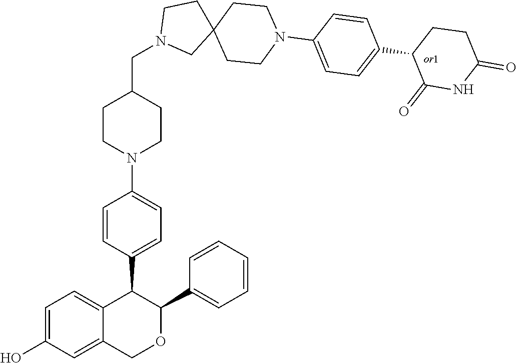 [SMILES: O=C1CC[C@@H](c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1] |
| I-391 | 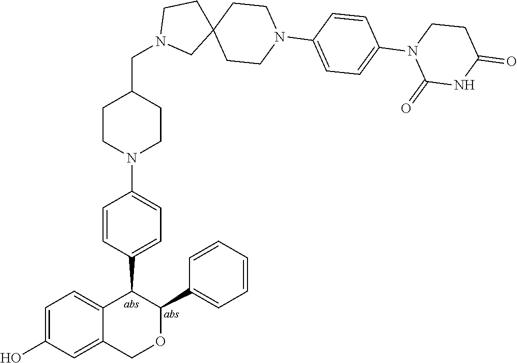 [SMILES: O=C1CCN(c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1] |
| I-392 | 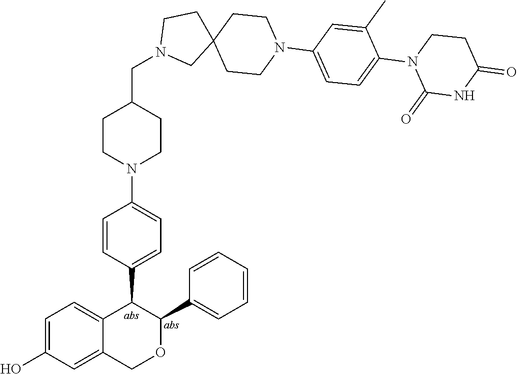 [SMILES: Cc1cc(N2CCC3(CCN(CC4CCN(c5ccc([C@@H]6c7ccc(O)cc7CO[C@@H]6c6ccccc6)cc5)CC4)C3)CC2)ccc1N1CCC(=O)NC1=O] |
| I-393 | 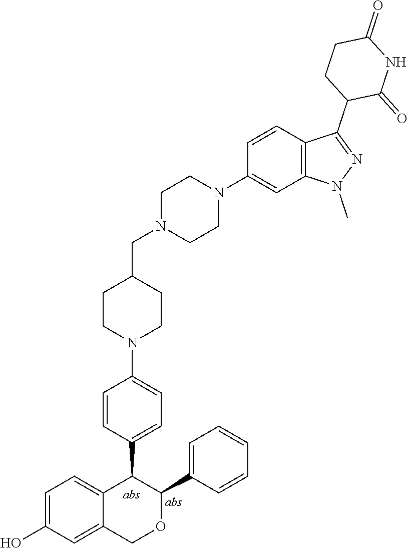 [SMILES: Cn1nc(C2CCC(=O)NC2=O)c2ccc(N3CCN(CC4CCN(c5ccc([C@@H]6c7ccc(O)cc7CO[C@@H]6c6ccccc6)cc5)CC4)CC3)cc21] |
| I-394 | 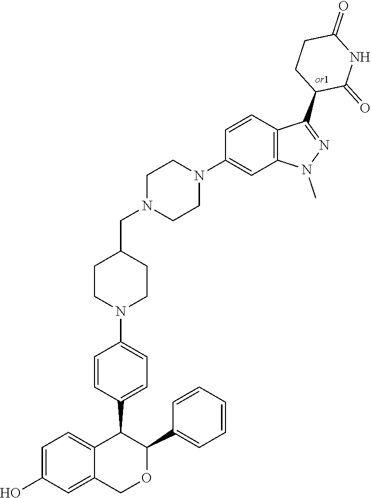 [SMILES: Cn1nc([C@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21] |
| I-395 | 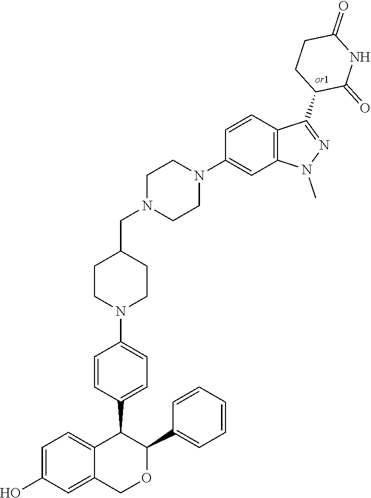 [SMILES: Cn1nc([C@@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21] |
| I-396 | 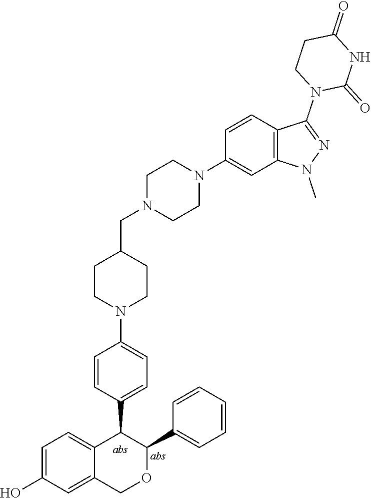 [SMILES: Cn1nc(N2CCC(=O)NC2=O)c2ccc(N3CCN(CC4CCN(c5ccc([C@@H]6c7ccc(O)cc7CO[C@@H]6c6ccccc6)cc5)CC4)CC3)cc21] |
| I-397 | 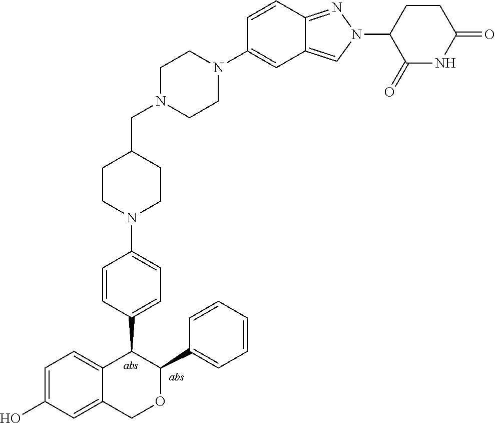 [SMILES: O=C1CCC(n2cc3cc(N4CCN(CC5CCN(c6ccc([C@@H]7c8ccc(O)cc8CO[C@@H]7c7ccccc7)cc6)CC5)CC4)ccc3n2)C(=O)N1] |
| I-398 | 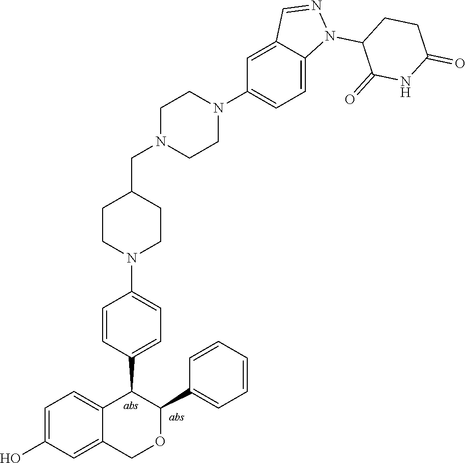 [SMILES: O=C1CCC(n2ncc3cc(N4CCN(CC5CCN(c6ccc([C@@H]7c8ccc(O)cc8CO[C@@H]7c7ccccc7)cc6)CC5)CC4)ccc32)C(=O)N1] |
| I-399 | 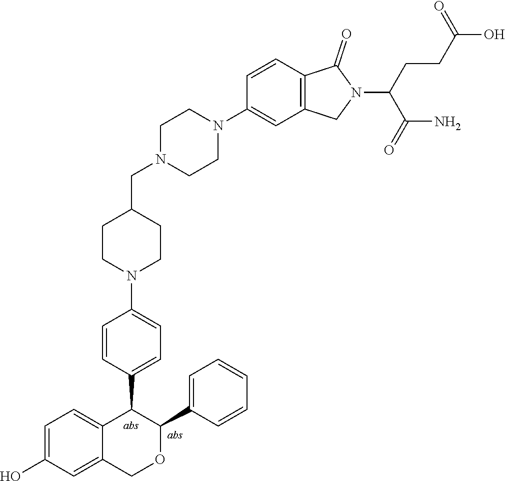 [SMILES: NC(=O)C(CCC(=O)O)N1Cc2cc(N3CCN(CC4CCN(c5ccc([C@@H]6c7ccc(O)cc7CO[C@@H]6c6ccccc6)cc5)CC4)CC3)ccc2C1=O] |
| I-400 | 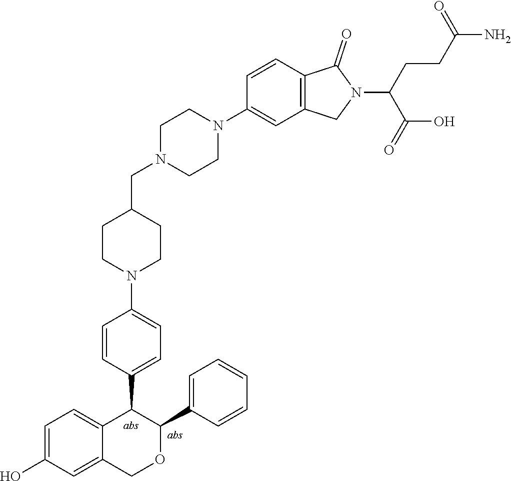 [SMILES: NC(=O)CCC(C(=O)O)N1Cc2cc(N3CCN(CC4CCN(c5ccc([C@@H]6c7ccc(O)cc7CO[C@@H]6c6ccccc6)cc5)CC4)CC3)ccc2C1=O] |
| I-401 | 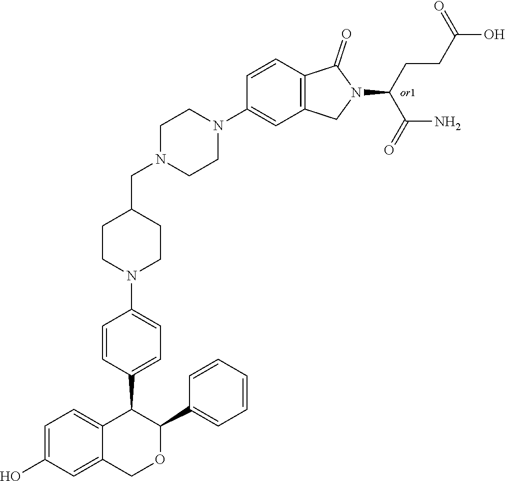 [SMILES: NC(=O)[C@H](CCC(=O)O)N1Cc2cc(N3CCN(CC4CCN(c5ccc([C@@H]6c7ccc(O)cc7CO[C@@H]6c6ccccc6)cc5)CC4)CC3)ccc2C1=O] |
| I-402 | 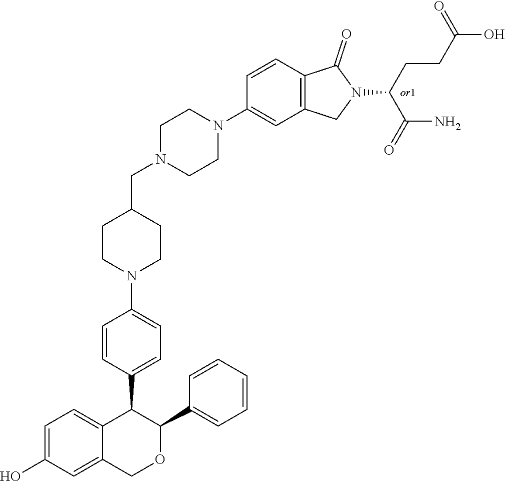 [SMILES: NC(=O)[C@@H](CCC(=O)O)N1Cc2cc(N3CCN(CC4CCN(c5ccc([C@@H]6c7ccc(O)cc7CO[C@@H]6c6ccccc6)cc5)CC4)CC3)ccc2C1=O] |
| I-403 | 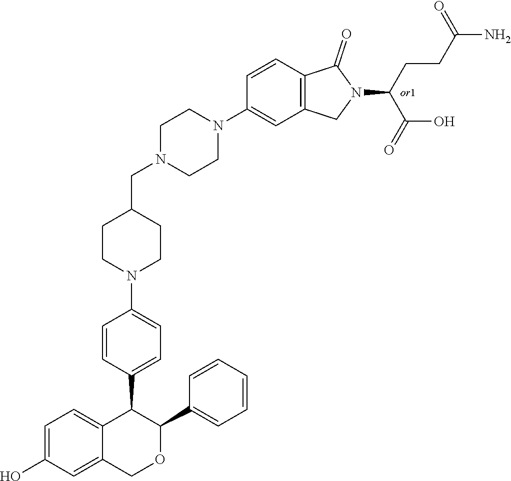 [SMILES: NC(=O)CC[C@@H](C(=O)O)N1Cc2cc(N3CCN(CC4CCN(c5ccc([C@@H]6c7ccc(O)cc7CO[C@@H]6c6ccccc6)cc5)CC4)CC3)ccc2C1=O] |
| I-404 | 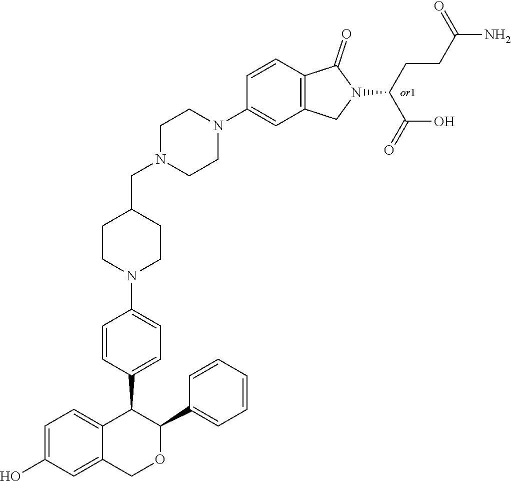 [SMILES: NC(=O)CC[C@H](C(=O)O)N1Cc2cc(N3CCN(CC4CCN(c5ccc([C@@H]6c7ccc(O)cc7CO[C@@H]6c6ccccc6)cc5)CC4)CC3)ccc2C1=O] |
| I-405 | 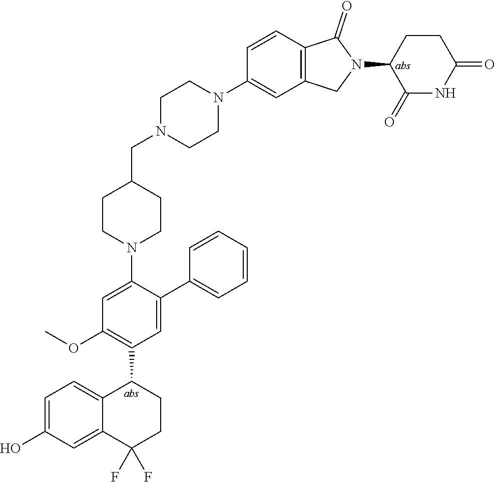 [SMILES: COc1cc(N2CCC(CN3CCN(c4ccc5c(c4)CN([C@H]4CCC(=O)NC4=O)C5=O)CC3)CC2)c(-c2ccccc2)cc1[C@@H]1CCC(F)(F)c2cc(O)ccc21] |
| I-406 | 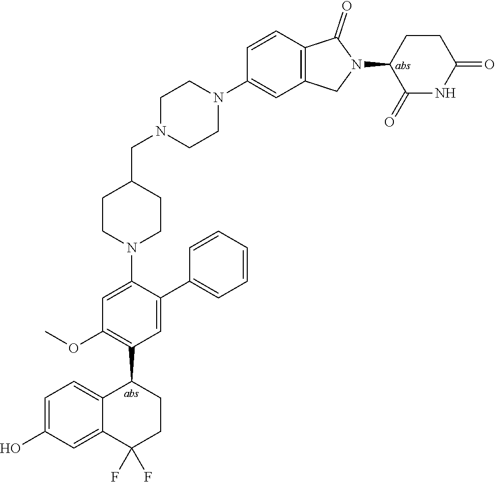 [SMILES: COc1cc(N2CCC(CN3CCN(c4ccc5c(c4)CN([C@H]4CCC(=O)NC4=O)C5=O)CC3)CC2)c(-c2ccccc2)cc1[C@H]1CCC(F)(F)c2cc(O)ccc21] |
| I-407 | 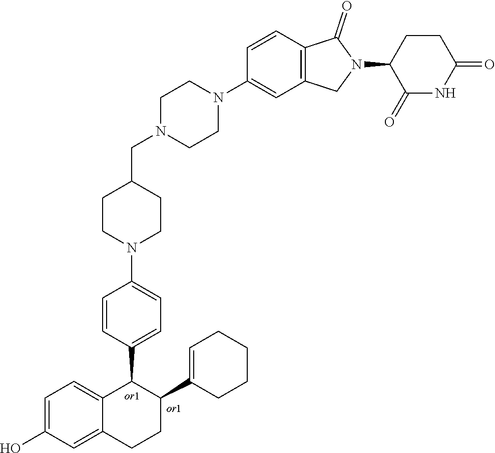 [SMILES: O=C1CC[C@H](N2Cc3cc(N4CCN(CC5CCN(c6ccc([C@@H]7c8ccc(O)cc8CC[C@@H]7C7=CCCCC7)cc6)CC5)CC4)ccc3C2=O)C(=O)N1] |
| I-408 | 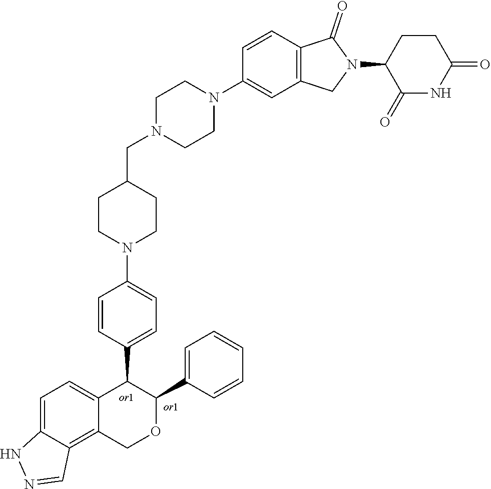 [SMILES: O=C1CC[C@H](N2Cc3cc(N4CCN(CC5CCN(c6ccc([C@@H]7c8ccc9[nH]ncc9c8CO[C@@H]7c7ccccc7)cc6)CC5)CC4)ccc3C2=O)C(=O)N1] |
| I-409 | 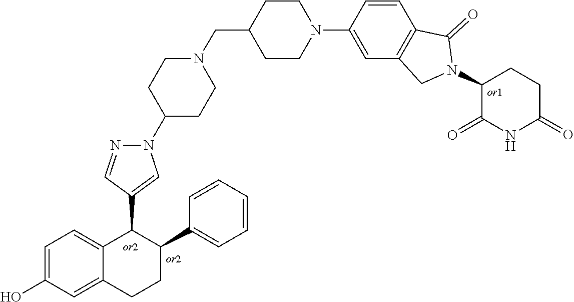 [SMILES: O=C1CC[C@H](N2Cc3cc(N4CCC(CN5CCC(n6cc([C@@H]7c8ccc(O)cc8CC[C@@H]7c7ccccc7)cn6)CC5)CC4)ccc3C2=O)C(=O)N1] |
| I-410 | 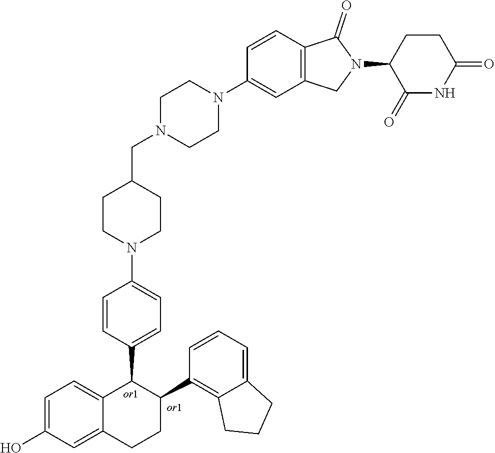 [SMILES: O=C1CC[C@H](N2Cc3cc(N4CCN(CC5CCN(c6ccc([C@@H]7c8ccc(O)cc8CC[C@@H]7c7cccc8c7CCC8)cc6)CC5)CC4)ccc3C2=O)C(=O)N1] |
| I-411 | 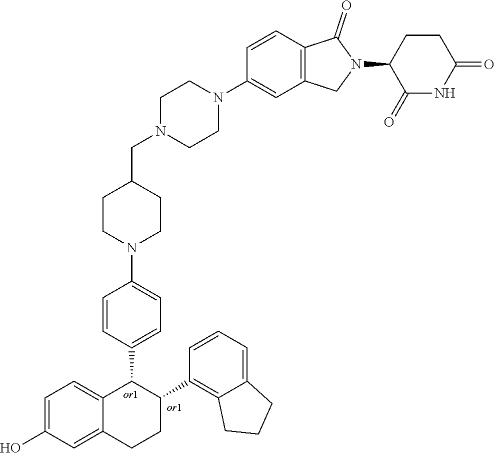 [SMILES: O=C1CC[C@H](N2Cc3cc(N4CCN(CC5CCN(c6ccc([C@H]7c8ccc(O)cc8CC[C@H]7c7cccc8c7CCC8)cc6)CC5)CC4)ccc3C2=O)C(=O)N1] |
| I-412 | 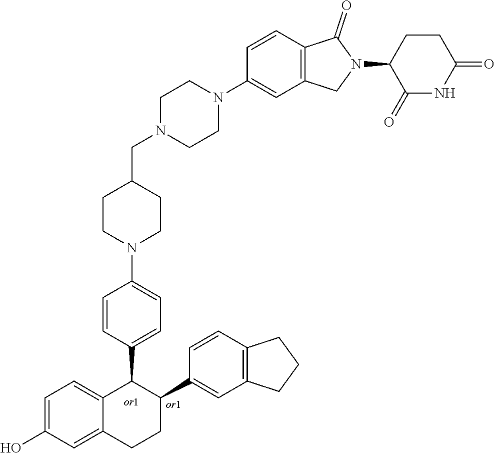 [SMILES: O=C1CC[C@H](N2Cc3cc(N4CCN(CC5CCN(c6ccc([C@@H]7c8ccc(O)cc8CC[C@@H]7c7ccc8c(c7)CCC8)cc6)CC5)CC4)ccc3C2=O)C(=O)N1] |
| I-413 | 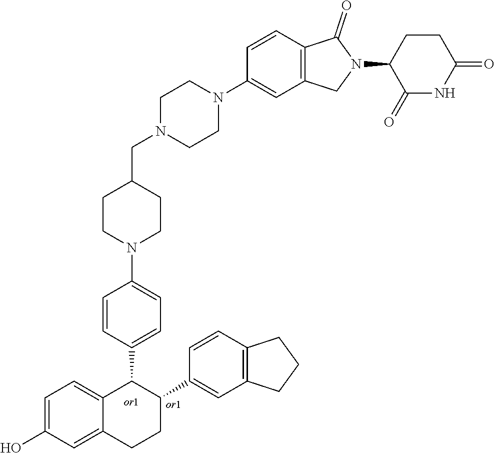 [SMILES: O=C1CC[C@H](N2Cc3cc(N4CCN(CC5CCN(c6ccc([C@H]7c8ccc(O)cc8CC[C@H]7c7ccc8c(c7)CCC8)cc6)CC5)CC4)ccc3C2=O)C(=O)N1] |
| I-414 | 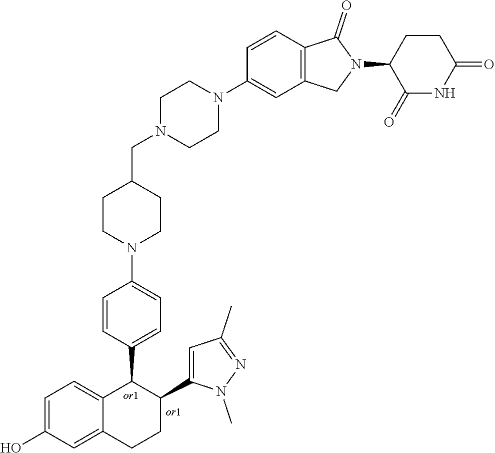 [SMILES: Cc1cc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)n(C)n1] |
| I-415 | 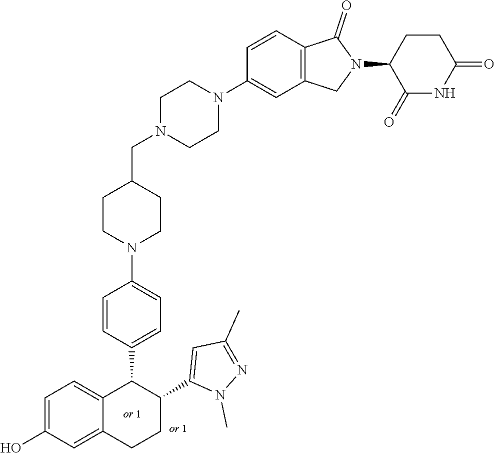 [SMILES: Cc1cc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n(C)n1] |
| I-416 | 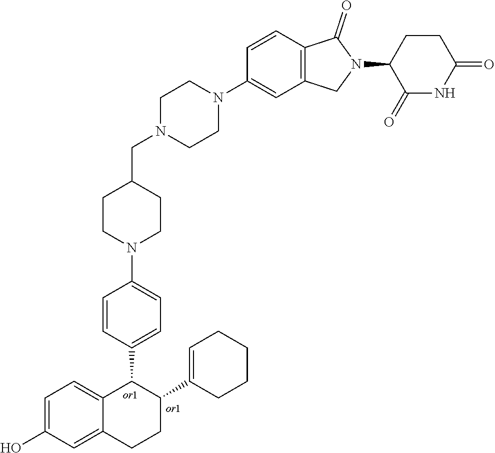 [SMILES: O=C1CC[C@H](N2Cc3cc(N4CCN(CC5CCN(c6ccc([C@H]7c8ccc(O)cc8CC[C@H]7C7=CCCCC7)cc6)CC5)CC4)ccc3C2=O)C(=O)N1] |
| I-417 | 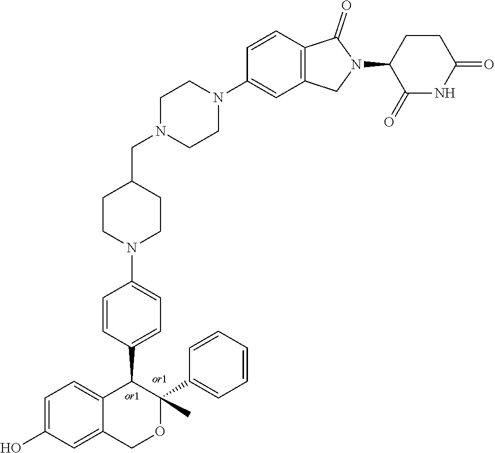 [SMILES: C[C@@]1(c2ccccc2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-418 | 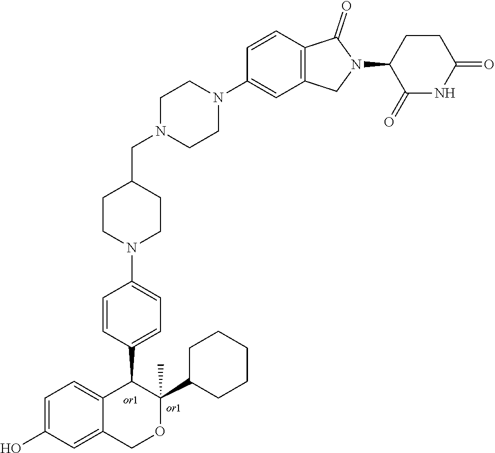 [SMILES: C[C@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1] |
| I-419 | 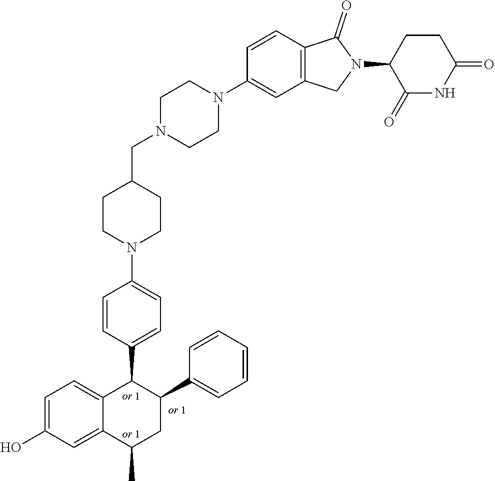 [SMILES: C[C@@H]1C[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-420 | 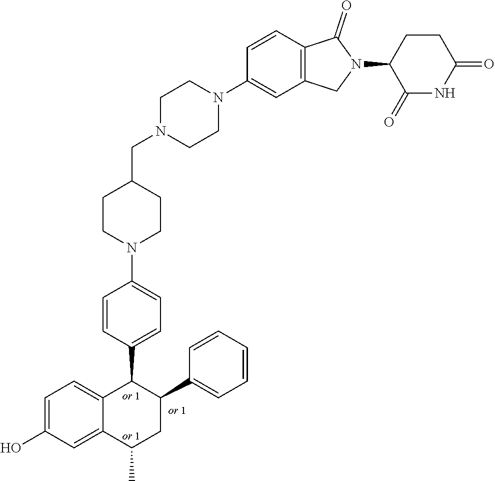 [SMILES: C[C@H]1C[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21] |
| I-421 | 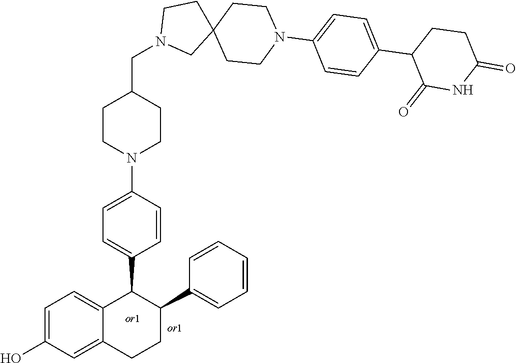 [SMILES: O=C1CCC(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1] |
| I-422 | 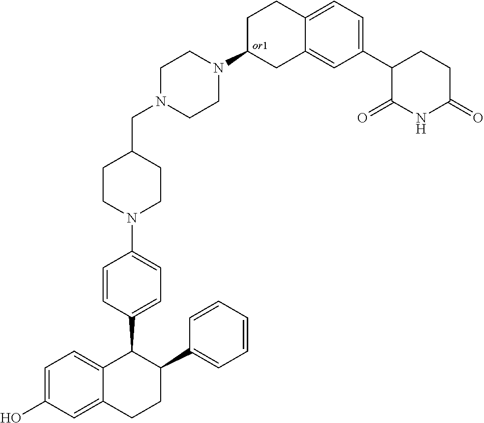 [SMILES: O=C1CCC(c2ccc3c(c2)C[C@@H](N2CCN(CC4CCN(c5ccc([C@@H]6c7ccc(O)cc7CC[C@@H]6c6ccccc6)cc5)CC4)CC2)CC3)C(=O)N1] |
| I-423 | 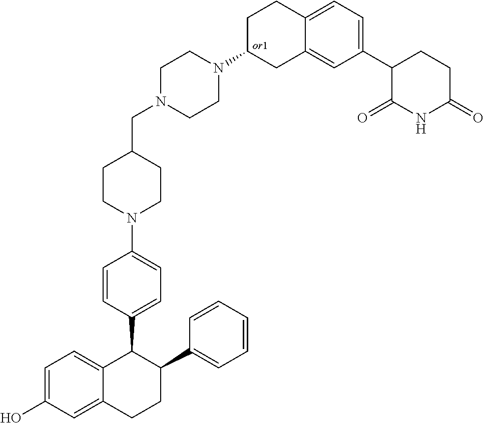 [SMILES: O=C1CCC(c2ccc3c(c2)C[C@H](N2CCN(CC4CCN(c5ccc([C@@H]6c7ccc(O)cc7CC[C@@H]6c6ccccc6)cc5)CC4)CC2)CC3)C(=O)N1] |

“‡” denotes compounds for which the initial stereochemistry was arbitrarily assigned, as indicated by the previous “or1” designation at the respective stereocenters. Current stereochemistry assignment has been confirmed by independent experiments, as indicated by the “abs” designation at the respective stereocenters.

TABLE 2
Additional Representative Compounds of the Disclosure.

| Compound | |
|---|---|
| No. | Structure |
| II-1 | 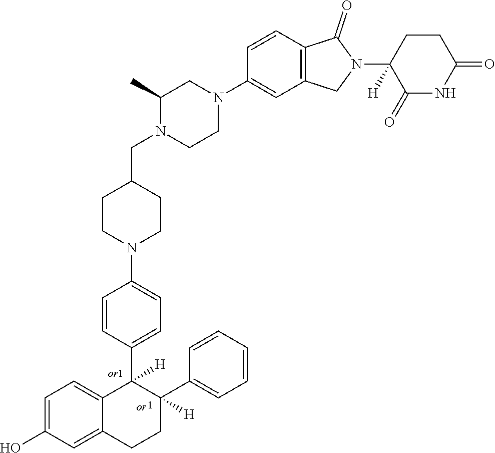 [SMILES: C[C@H]1CN(c2ccc3c(c2)CN([C@H]2CCC(=O)NC2=O)C3=O)CCN1CC1CCN(c2ccc([C@@H]3c4ccc(O)cc4CC[C@@H]3c3ccccc3)cc2)CC1] |
| II-2 | 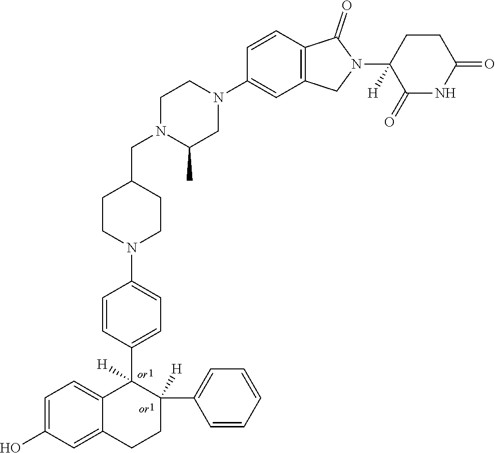 [SMILES: C[C@@H]1CN(c2ccc3c(c2)CN([C@H]2CCC(=O)NC2=O)C3=O)CCN1CC1CCN(c2ccc([C@@H]3c4ccc(O)cc4CC[C@@H]3c3ccccc3)cc2)CC1] |
| II-3 | 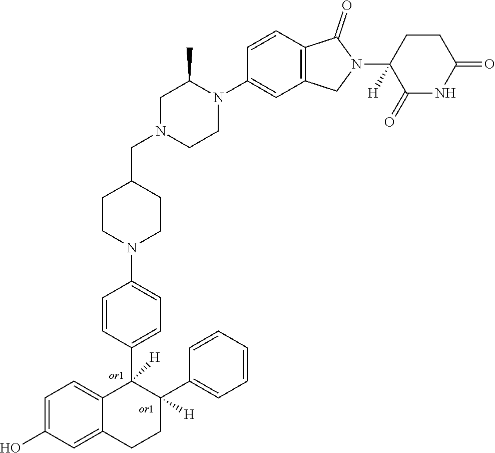 [SMILES: C[C@@H]1CN(CC2CCN(c3ccc([C@@H]4c5ccc(O)cc5CC[C@@H]4c4ccccc4)cc3)CC2)CCN1c1ccc2c(c1)CN([C@H]1CCC(=O)NC1=O)C2=O] |
| II-4 | 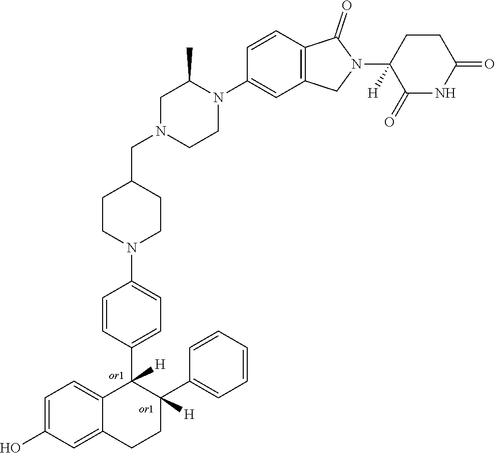 [SMILES: C[C@@H]1CN(CC2CCN(c3ccc([C@H]4c5ccc(O)cc5CC[C@H]4c4ccccc4)cc3)CC2)CCN1c1ccc2c(c1)CN([C@H]1CCC(=O)NC1=O)C2=O] |
| II-5 | 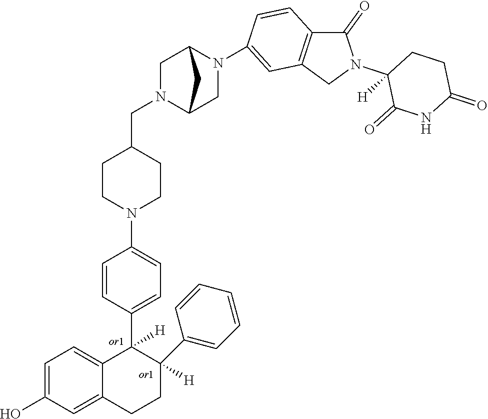 [SMILES: O=C1CC[C@H](N2Cc3cc(N4C[C@H]5C[C@@H]4CN5CC4CCN(c5ccc([C@@H]6c7ccc(O)cc7CC[C@@H]6c6ccccc6)cc5)CC4)ccc3C2=O)C(=O)N1] |
| II-6 | 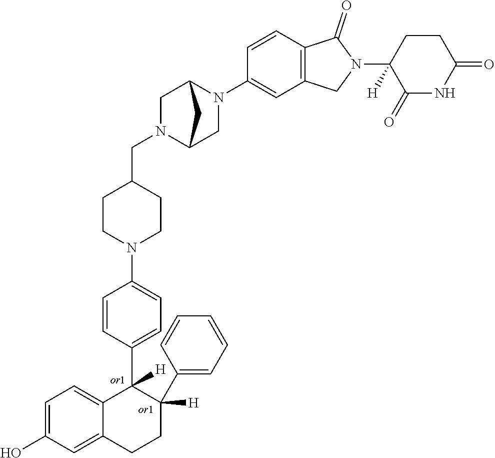 [SMILES: O=C1CC[C@H](N2Cc3cc(N4C[C@H]5C[C@@H]4CN5CC4CCN(c5ccc([C@H]6c7ccc(O)cc7CC[C@H]6c6ccccc6)cc5)CC4)ccc3C2=O)C(=O)N1] |
| II-7 | 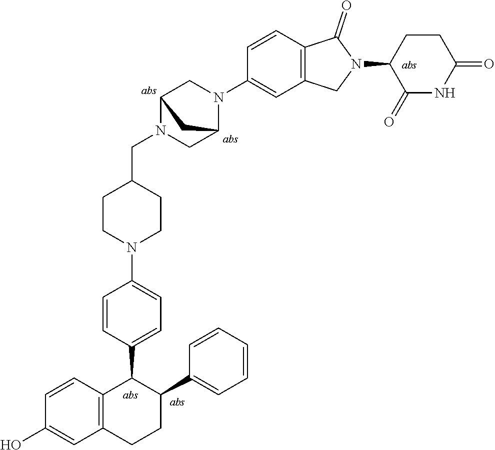 [SMILES: O=C1CC[C@H](N2Cc3cc(N4C[C@@H]5C[C@H]4CN5CC4CCN(c5ccc([C@@H]6c7ccc(O)cc7CC[C@@H]6c6ccccc6)cc5)CC4)ccc3C2=O)C(=O)N1] |
| II-8 | 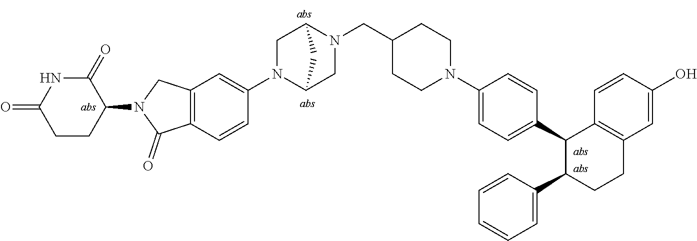 [SMILES: O=C1CC[C@H](N2Cc3cc(N4C[C@@H]5C[C@H]4CN5CC4CCN(c5ccc([C@H]6c7ccc(O)cc7CC[C@H]6c6ccccc6)cc5)CC4)ccc3C2=O)C(=O)N1] |
| II-9 | 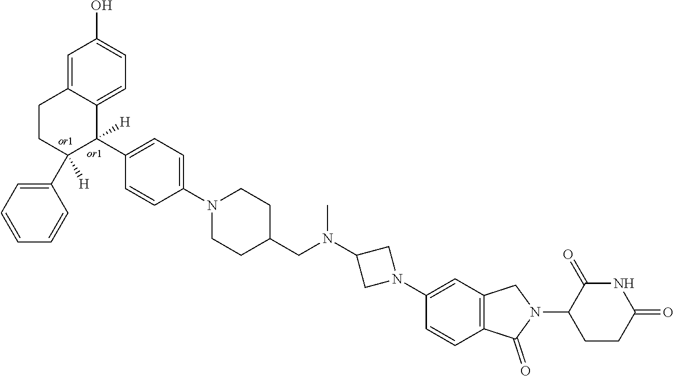 [SMILES: CN(CC1CCN(c2ccc([C@@H]3c4ccc(O)cc4CC[C@@H]3c3ccccc3)cc2)CC1)C1CN(c2ccc3c(c2)CN(C2CCC(=O)NC2=O)C3=O)C1] |
| II-10 | 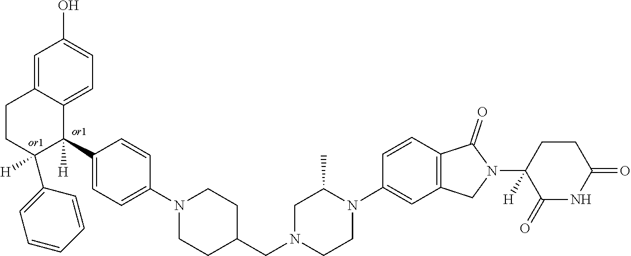 [SMILES: C[C@H]1CN(CC2CCN(c3ccc([C@@H]4c5ccc(O)cc5CC[C@@H]4c4ccccc4)cc3)CC2)CCN1c1ccc2c(c1)CN([C@H]1CCC(=O)NC1=O)C2=O] |
| II-11 | 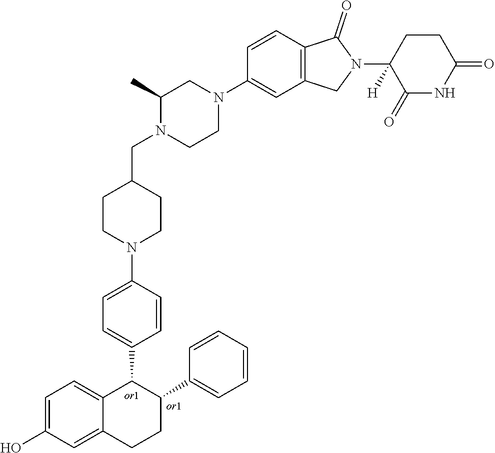 [SMILES: C[C@H]1CN(c2ccc3c(c2)CN([C@H]2CCC(=O)NC2=O)C3=O)CCN1CC1CCN(c2ccc([C@H]3c4ccc(O)cc4CC[C@H]3c3ccccc3)cc2)CC1] |
| II-12 | 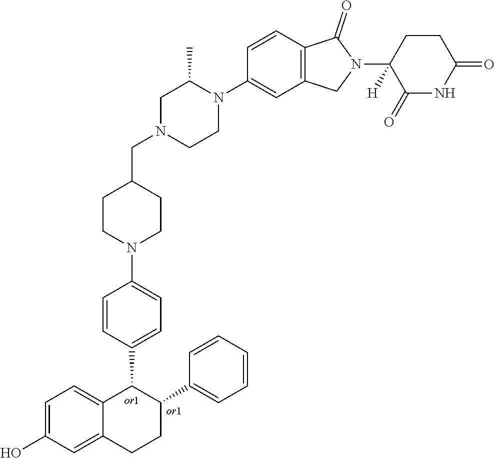 [SMILES: C[C@H]1CN(CC2CCN(c3ccc([C@H]4c5ccc(O)cc5CC[C@H]4c4ccccc4)cc3)CC2)CCN1c1ccc2c(c1)CN([C@H]1CCC(=O)NC1=O)C2=O] |
| II-13 | 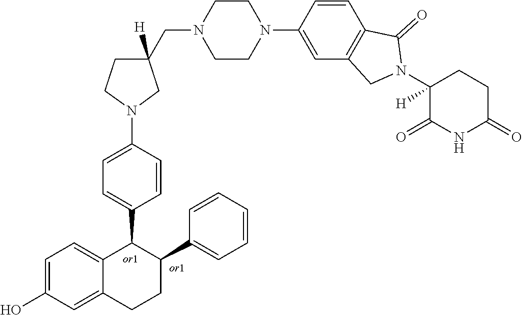 [SMILES: O=C1CC[C@H](N2Cc3cc(N4CCN(C[C@H]5CCN(c6ccc([C@@H]7c8ccc(O)cc8CC[C@@H]7c7ccccc7)cc6)C5)CC4)ccc3C2=O)C(=O)N1] |
| II-14 | 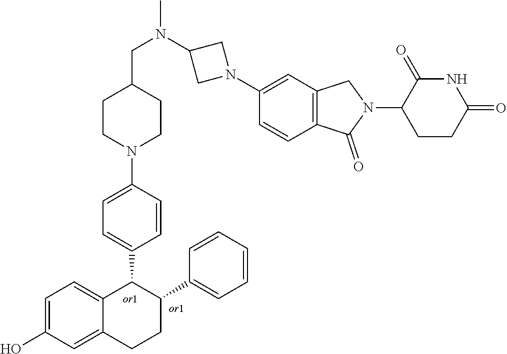 [SMILES: CN(CC1CCN(c2ccc([C@H]3c4ccc(O)cc4CC[C@H]3c3ccccc3)cc2)CC1)C1CN(c2ccc3c(c2)CN(C2CCC(=O)NC2=O)C3=O)C1] |
| II-15 | 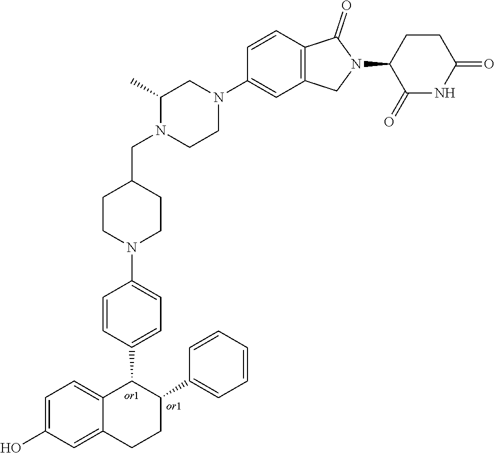 [SMILES: C[C@@H]1CN(c2ccc3c(c2)CN([C@H]2CCC(=O)NC2=O)C3=O)CCN1CC1CCN(c2ccc([C@H]3c4ccc(O)cc4CC[C@H]3c3ccccc3)cc2)CC1] |
| II-16 | 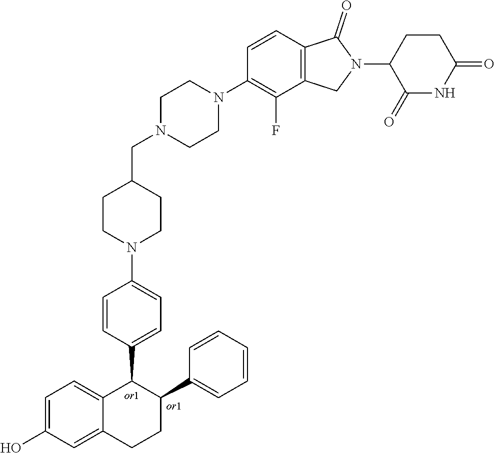 [SMILES: O=C1CCC(N2Cc3c(ccc(N4CCN(CC5CCN(c6ccc([C@@H]7c8ccc(O)cc8CC[C@@H]7c7ccccc7)cc6)CC5)CC4)c3F)C2=O)C(=O)N1] |
| II-17 | 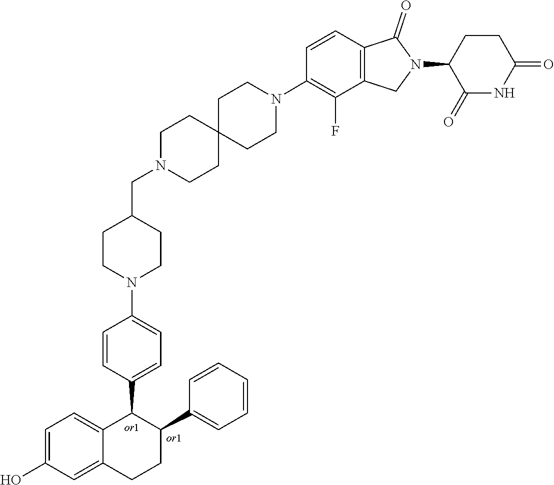 [SMILES: O=C1CC[C@H](N2Cc3c(ccc(N4CCC5(CCN(CC6CCN(c7ccc([C@@H]8c9ccc(O)cc9CC[C@@H]8c8ccccc8)cc7)CC6)CC5)CC4)c3F)C2=O)C(=O)N1] |
| II-18 | 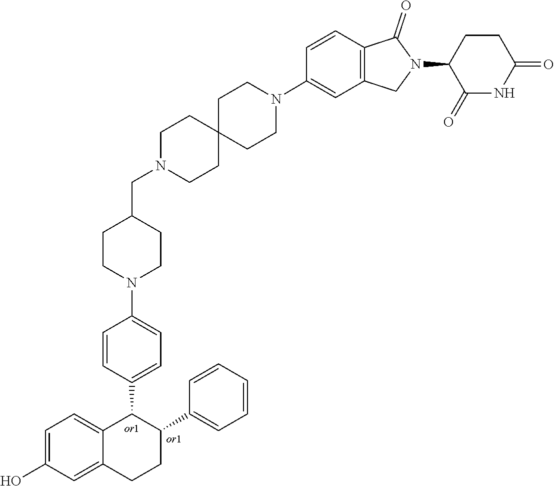 [SMILES: O=C1CC[C@H](N2Cc3cc(N4CCC5(CCN(CC6CCN(c7ccc([C@H]8c9ccc(O)cc9CC[C@H]8c8ccccc8)cc7)CC6)CC5)CC4)ccc3C2=O)C(=O)N1] |
| II-19 | 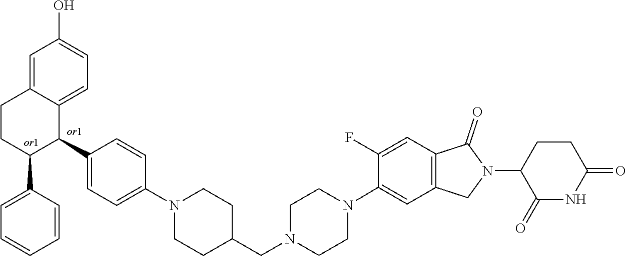 [SMILES: O=C1CCC(N2Cc3cc(N4CCN(CC5CCN(c6ccc([C@@H]7c8ccc(O)cc8CC[C@@H]7c7ccccc7)cc6)CC5)CC4)c(F)cc3C2=O)C(=O)N1] |
| II-20 | 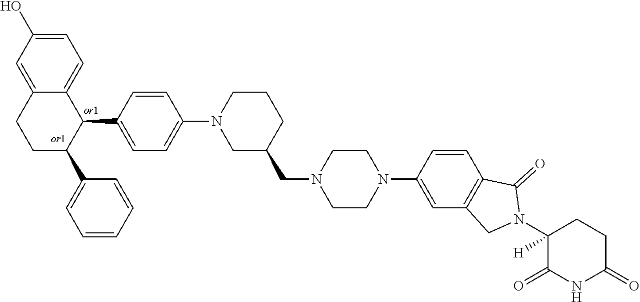 [SMILES: O=C1CC[C@H](N2Cc3cc(N4CCN(C[C@@H]5CCCN(c6ccc([C@@H]7c8ccc(O)cc8CC[C@@H]7c7ccccc7)cc6)C5)CC4)ccc3C2=O)C(=O)N1] |
| II-21 | 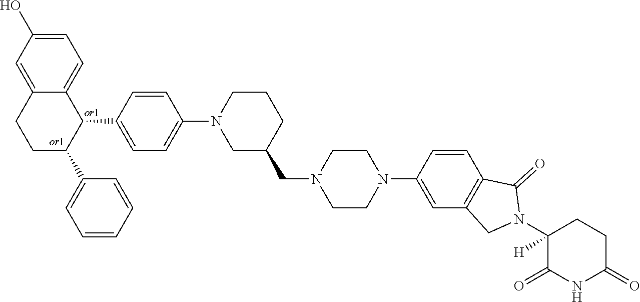 [SMILES: O=C1CC[C@H](N2Cc3cc(N4CCN(C[C@@H]5CCCN(c6ccc([C@H]7c8ccc(O)cc8CC[C@H]7c7ccccc7)cc6)C5)CC4)ccc3C2=O)C(=O)N1] |
| II-22 | 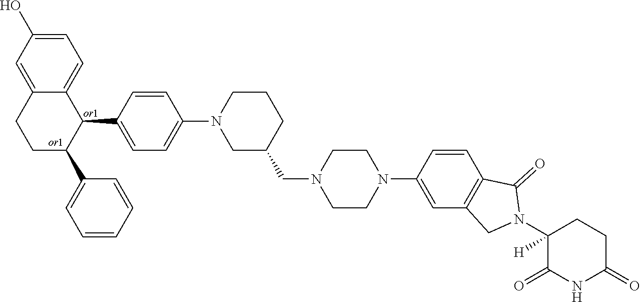 [SMILES: O=C1CC[C@H](N2Cc3cc(N4CCN(C[C@H]5CCCN(c6ccc([C@@H]7c8ccc(O)cc8CC[C@@H]7c7ccccc7)cc6)C5)CC4)ccc3C2=O)C(=O)N1] |
| II-23 | 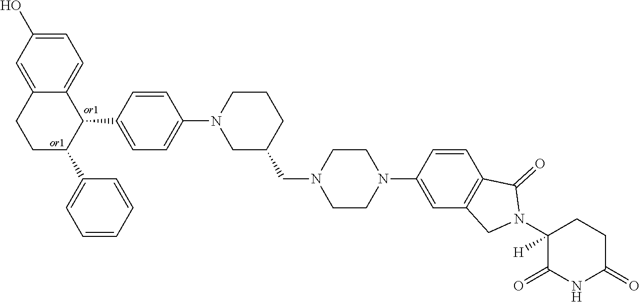 [SMILES: O=C1CC[C@H](N2Cc3cc(N4CCN(C[C@H]5CCCN(c6ccc([C@H]7c8ccc(O)cc8CC[C@H]7c7ccccc7)cc6)C5)CC4)ccc3C2=O)C(=O)N1] |
| II-24 | 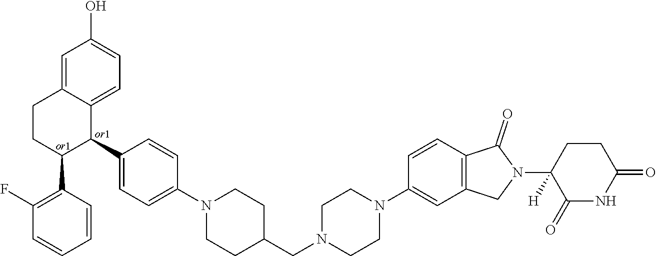 [SMILES: O=C1CC[C@H](N2Cc3cc(N4CCN(CC5CCN(c6ccc([C@@H]7c8ccc(O)cc8CC[C@@H]7c7ccccc7F)cc6)CC5)CC4)ccc3C2=O)C(=O)N1] |
| II-25 | 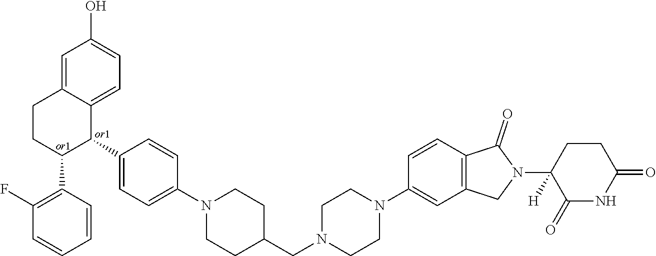 [SMILES: O=C1CC[C@H](N2Cc3cc(N4CCN(CC5CCN(c6ccc([C@H]7c8ccc(O)cc8CC[C@H]7c7ccccc7F)cc6)CC5)CC4)ccc3C2=O)C(=O)N1] |
| II-26 | 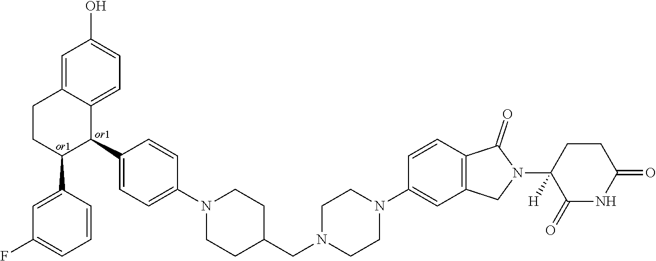 [SMILES: O=C1CC[C@H](N2Cc3cc(N4CCN(CC5CCN(c6ccc([C@@H]7c8ccc(O)cc8CC[C@@H]7c7cccc(F)c7)cc6)CC5)CC4)ccc3C2=O)C(=O)N1] |
| II-27 | 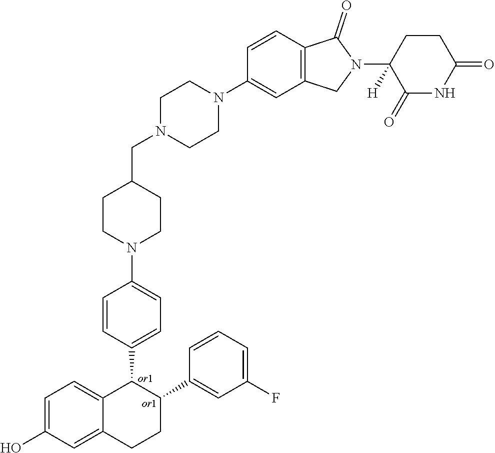 [SMILES: O=C1CC[C@H](N2Cc3cc(N4CCN(CC5CCN(c6ccc([C@H]7c8ccc(O)cc8CC[C@H]7c7cccc(F)c7)cc6)CC5)CC4)ccc3C2=O)C(=O)N1] |
| II-28 | 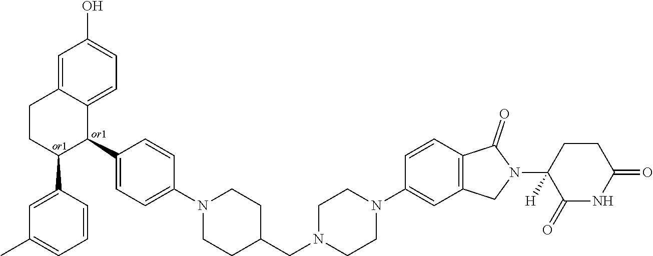 [SMILES: Cc1cccc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)c1] |
| II-29 | 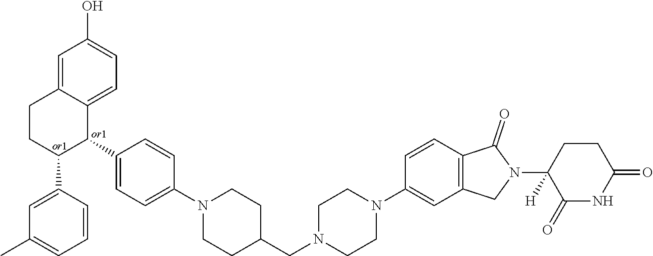 [SMILES: Cc1cccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)c1] |
| II-30 | 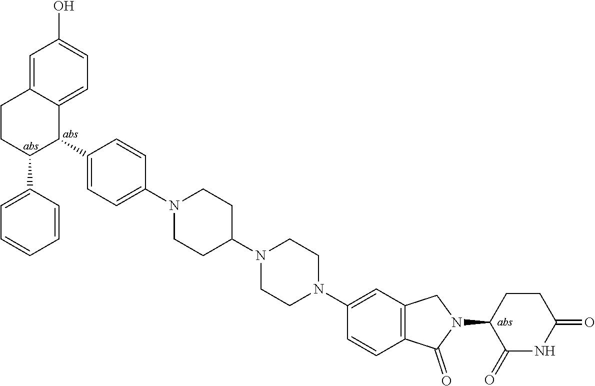 [SMILES: O=C1CC[C@H](N2Cc3cc(N4CCN(C5CCN(c6ccc([C@H]7c8ccc(O)cc8CC[C@H]7c7ccccc7)cc6)CC5)CC4)ccc3C2=O)C(=O)N1] |
| II-31 | 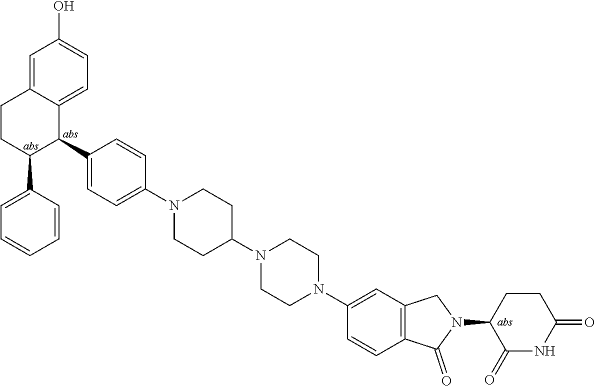 [SMILES: O=C1CC[C@H](N2Cc3cc(N4CCN(C5CCN(c6ccc([C@@H]7c8ccc(O)cc8CC[C@@H]7c7ccccc7)cc6)CC5)CC4)ccc3C2=O)C(=O)N1] |
| II-32 | 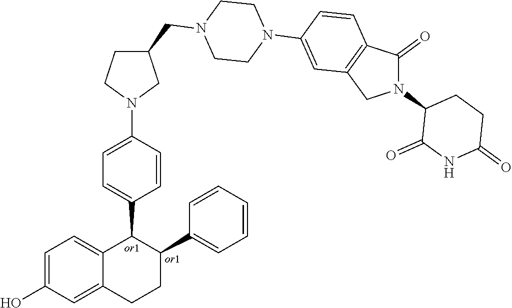 [SMILES: O=C1CC[C@H](N2Cc3cc(N4CCN(C[C@@H]5CCN(c6ccc([C@@H]7c8ccc(O)cc8CC[C@@H]7c7ccccc7)cc6)C5)CC4)ccc3C2=O)C(=O)N1] |
| II-33 | 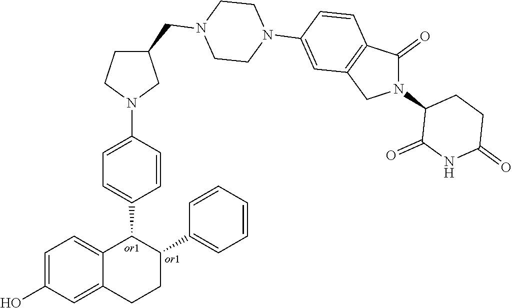 [SMILES: O=C1CC[C@H](N2Cc3cc(N4CCN(C[C@@H]5CCN(c6ccc([C@H]7c8ccc(O)cc8CC[C@H]7c7ccccc7)cc6)C5)CC4)ccc3C2=O)C(=O)N1] |
| II-34 | 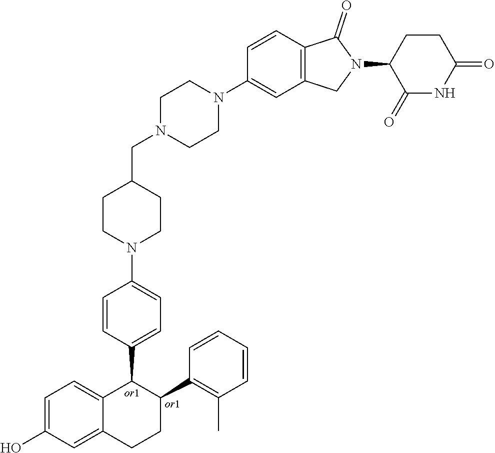 [SMILES: Cc1ccccc1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| II-35 | 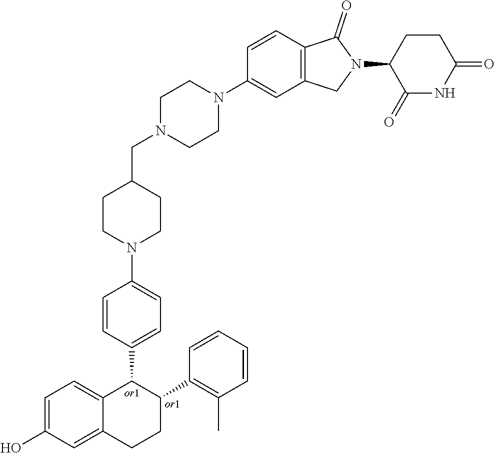 [SMILES: Cc1ccccc1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| II-36 | 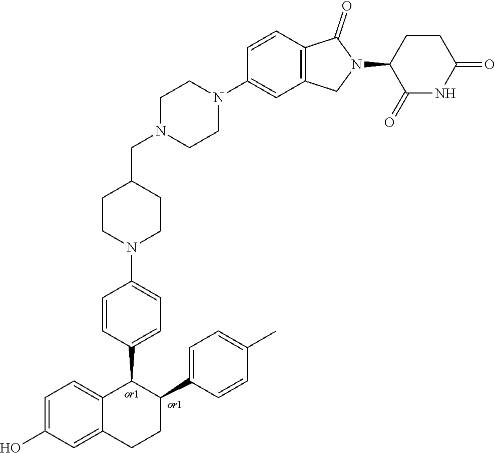 [SMILES: Cc1ccc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)cc1] |
| II-37 | 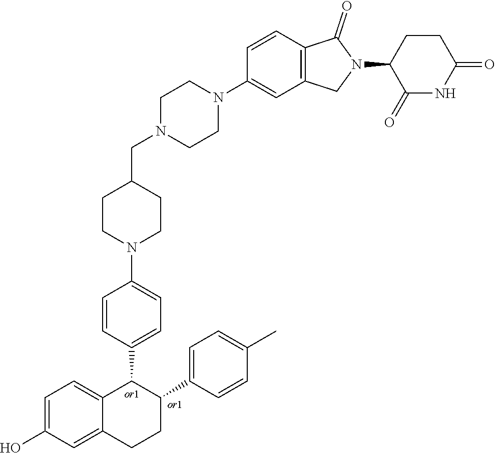 [SMILES: Cc1ccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1] |
| II-38 | 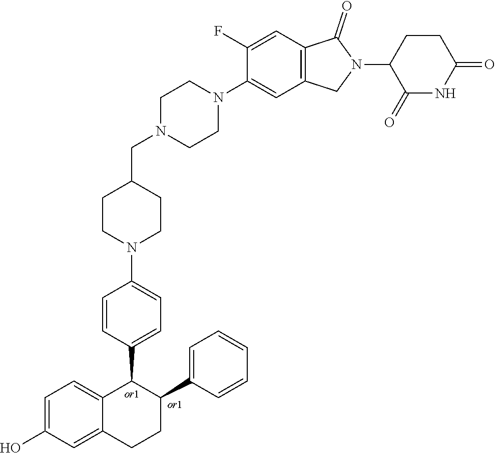 [SMILES: O=C1CCC(N2Cc3cc(N4CCN(CC5CCN(c6ccc([C@@H]7c8ccc(O)cc8CC[C@@H]7c7ccccc7)cc6)CC5)CC4)c(F)cc3C2=O)C(=O)N1] |
| II-39 | 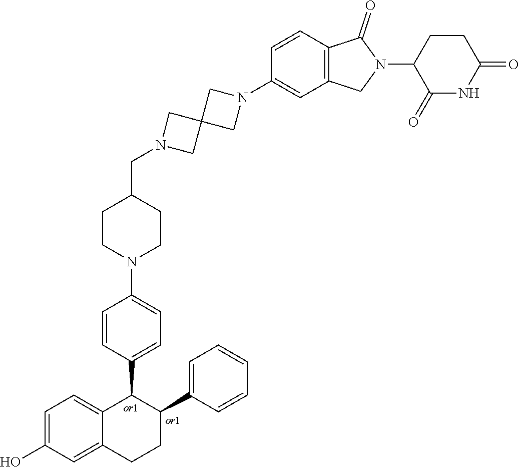 [SMILES: O=C1CCC(N2Cc3cc(N4CC5(CN(CC6CCN(c7ccc([C@@H]8c9ccc(O)cc9CC[C@@H]8c8ccccc8)cc7)CC6)C5)C4)ccc3C2=O)C(=O)N1] |
| II-40 | 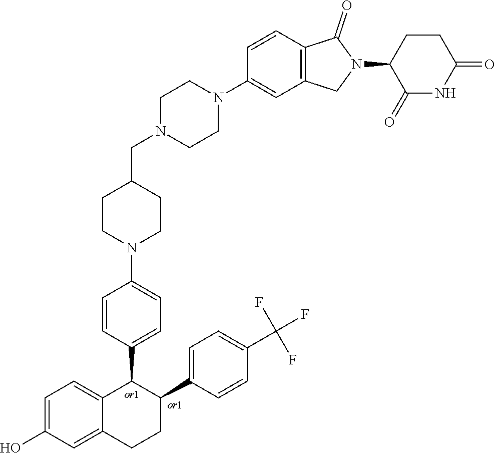 [SMILES: O=C1CC[C@H](N2Cc3cc(N4CCN(CC5CCN(c6ccc([C@@H]7c8ccc(O)cc8CC[C@@H]7c7ccc(C(F)(F)F)cc7)cc6)CC5)CC4)ccc3C2=O)C(=O)N1] |
| II-41 | 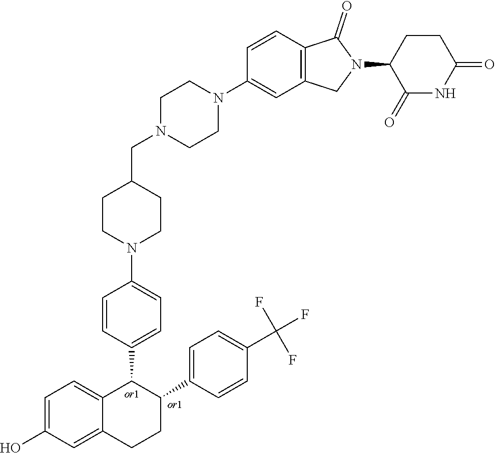 [SMILES: O=C1CC[C@H](N2Cc3cc(N4CCN(CC5CCN(c6ccc([C@H]7c8ccc(O)cc8CC[C@H]7c7ccc(C(F)(F)F)cc7)cc6)CC5)CC4)ccc3C2=O)C(=O)N1] |
| II-42 | 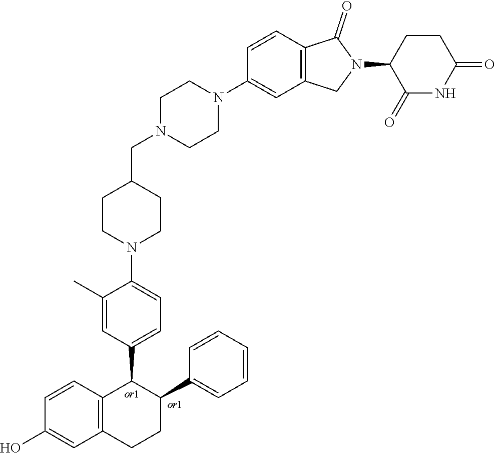 [SMILES: Cc1cc([C@@H]2c3ccc(O)cc3CC[C@@H]2c2ccccc2)ccc1N1CCC(CN2CCN(c3ccc4c(c3)CN([C@H]3CCC(=O)NC3=O)C4=O)CC2)CC1] |
| II-43 | 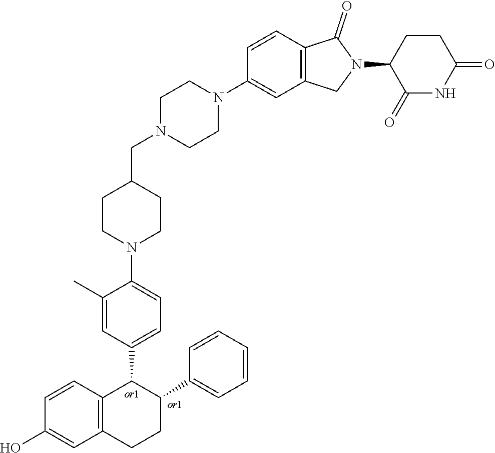 [SMILES: Cc1cc([C@H]2c3ccc(O)cc3CC[C@H]2c2ccccc2)ccc1N1CCC(CN2CCN(c3ccc4c(c3)CN([C@H]3CCC(=O)NC3=O)C4=O)CC2)CC1] |
| II-44 | 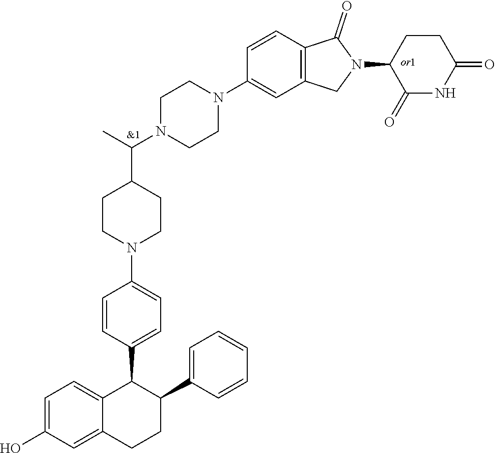 [SMILES: CC(C1CCN(c2ccc([C@@H]3c4ccc(O)cc4CC[C@@H]3c3ccccc3)cc2)CC1)N1CCN(c2ccc3c(c2)CN([C@H]2CCC(=O)NC2=O)C3=O)CC1] |
| II-47 | 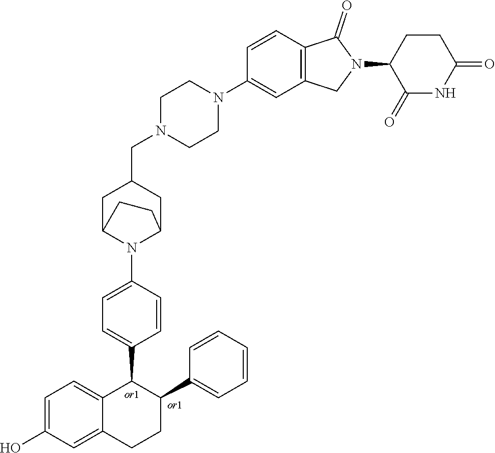 [SMILES: O=C1CC[C@H](N2Cc3cc(N4CCN(CC5CC6CCC(C5)N6c5ccc([C@@H]6c7ccc(O)cc7CC[C@@H]6c6ccccc6)cc5)CC4)ccc3C2=O)C(=O)N1] |
| II-48 | 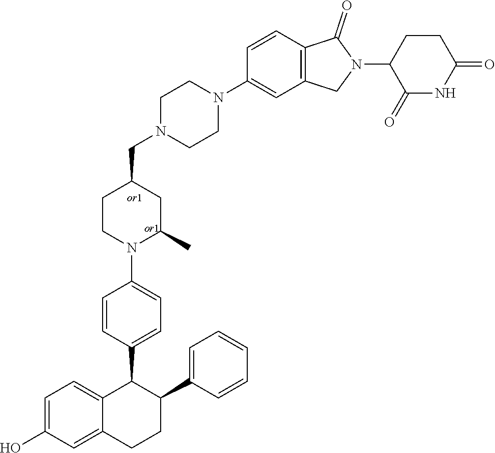 [SMILES: C[C@@H]1C[C@H](CN2CCN(c3ccc4c(c3)CN(C3CCC(=O)NC3=O)C4=O)CC2)CCN1c1ccc([C@@H]2c3ccc(O)cc3CC[C@@H]2c2ccccc2)cc1] |
| II-49 | 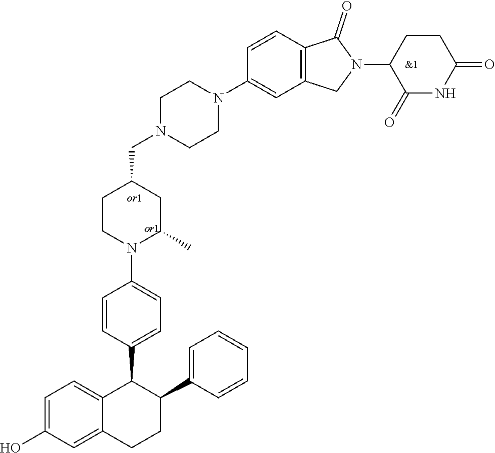 [SMILES: C[C@H]1C[C@@H](CN2CCN(c3ccc4c(c3)CN(C3CCC(=O)NC3=O)C4=O)CC2)CCN1c1ccc([C@@H]2c3ccc(O)cc3CC[C@@H]2c2ccccc2)cc1] |
| II-50 | 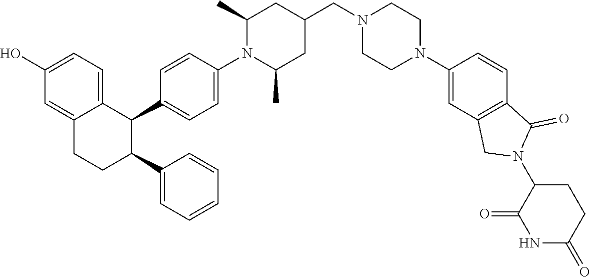 [SMILES: C[C@@H]1CC(CN2CCN(c3ccc4c(c3)CN(C3CCC(=O)NC3=O)C4=O)CC2)C[C@H](C)N1c1ccc([C@@H]2c3ccc(O)cc3CC[C@@H]2c2ccccc2)cc1] |
| II-51 | 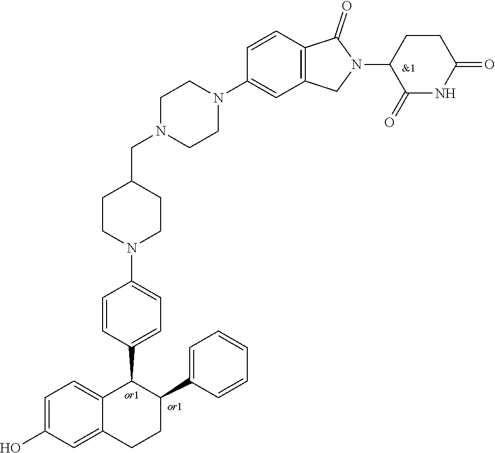 [SMILES: O=C1CCC(N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] |
| II-52 | 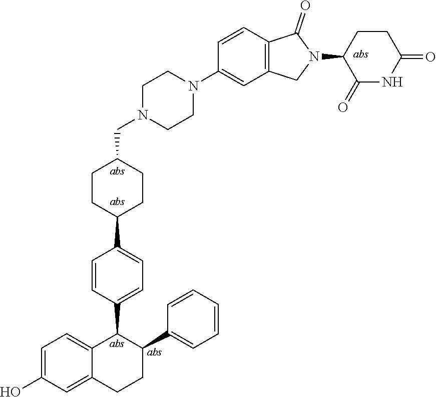 [SMILES: O=C1CC[C@H](N2Cc3cc(N4CCN(C[C@H]5CC[C@H](c6ccc([C@@H]7c8ccc(O)cc8CC[C@@H]7c7ccccc7)cc6)CC5)CC4)ccc3C2=O)C(=O)N1] |
| II-53 | 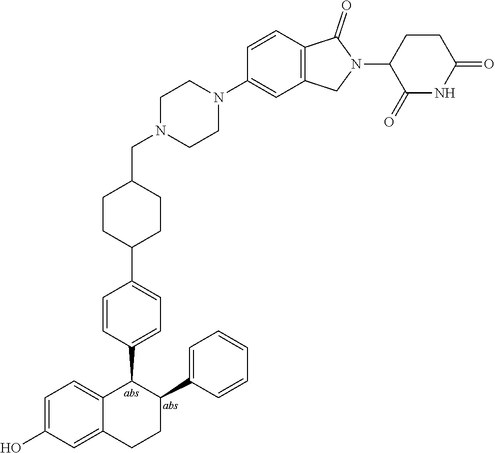 [SMILES: O=C1CCC(N2Cc3cc(N4CCN(CC5CCC(c6ccc([C@@H]7c8ccc(O)cc8CC[C@@H]7c7ccccc7)cc6)CC5)CC4)ccc3C2=O)C(=O)N1] |
| II-56 | 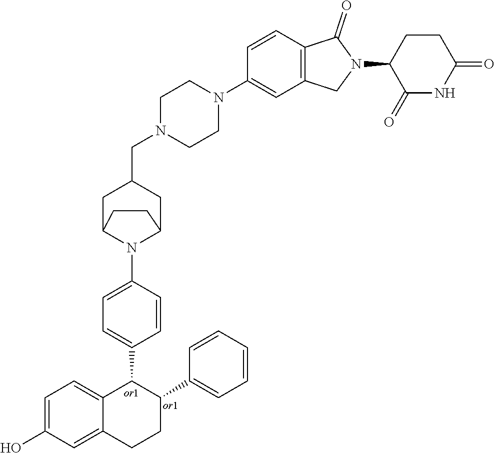 [SMILES: O=C1CC[C@H](N2Cc3cc(N4CCN(CC5CC6CCC(C5)N6c5ccc([C@H]6c7ccc(O)cc7CC[C@H]6c6ccccc6)cc5)CC4)ccc3C2=O)C(=O)N1] |
| II-57 | 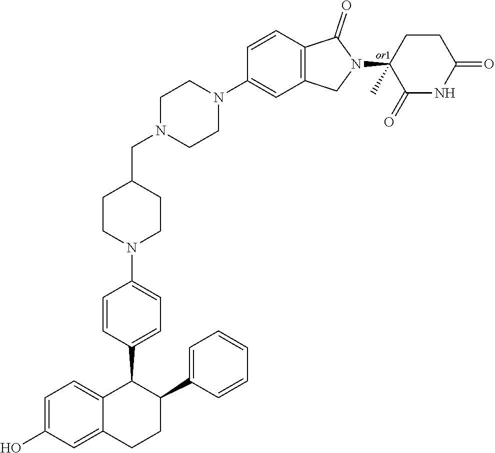 [SMILES: C[C@]1(N2Cc3cc(N4CCN(CC5CCN(c6ccc([C@@H]7c8ccc(O)cc8CC[C@@H]7c7ccccc7)cc6)CC5)CC4)ccc3C2=O)CCC(=O)NC1=O] |
| II-58 | 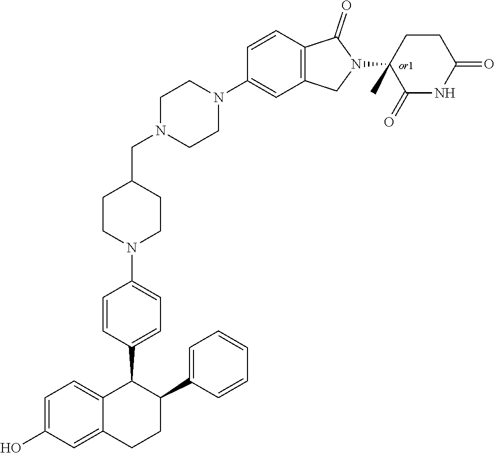 [SMILES: C[C@@]1(N2Cc3cc(N4CCN(CC5CCN(c6ccc([C@@H]7c8ccc(O)cc8CC[C@@H]7c7ccccc7)cc6)CC5)CC4)ccc3C2=O)CCC(=O)NC1=O] |
| II-59 | 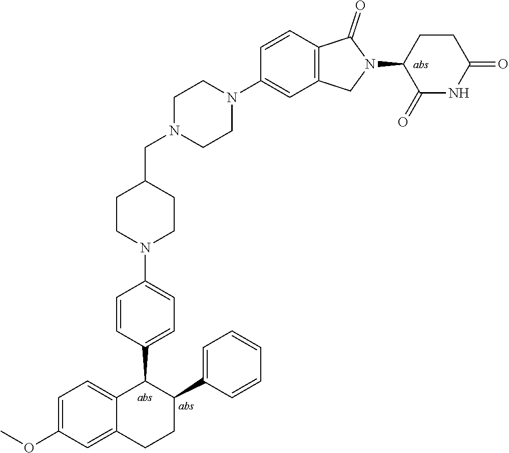 [SMILES: COc1ccc2c(c1)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1] |
| II-60 | 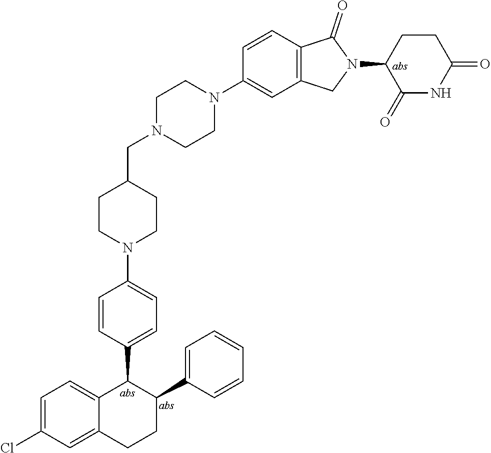 [SMILES: O=C1CC[C@H](N2Cc3cc(N4CCN(CC5CCN(c6ccc([C@@H]7c8ccc(Cl)cc8CC[C@@H]7c7ccccc7)cc6)CC5)CC4)ccc3C2=O)C(=O)N1] |
| II-61 | 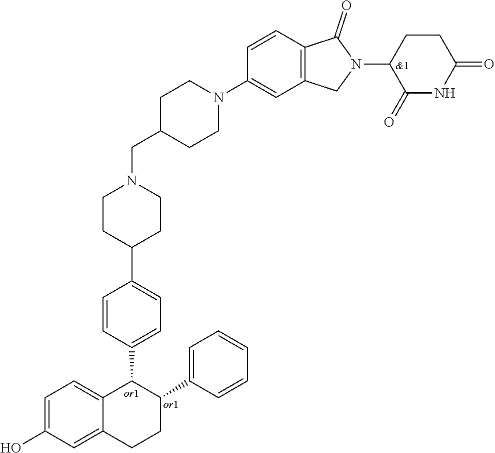 [SMILES: O=C1CCC(N2Cc3cc(N4CCC(CN5CCC(c6ccc([C@H]7c8ccc(O)cc8CC[C@H]7c7ccccc7)cc6)CC5)CC4)ccc3C2=O)C(=O)N1] |
| II-62 | 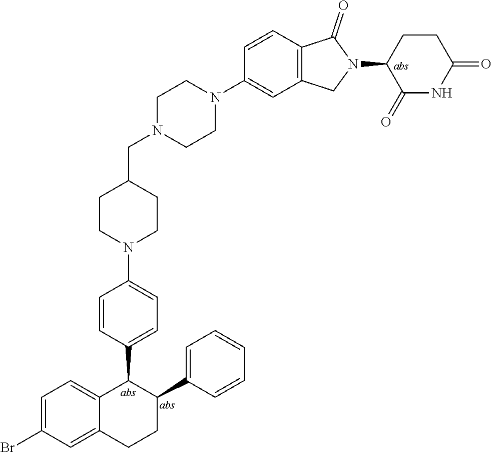 [SMILES: O=C1CC[C@H](N2Cc3cc(N4CCN(CC5CCN(c6ccc([C@@H]7c8ccc(Br)cc8CC[C@@H]7c7ccccc7)cc6)CC5)CC4)ccc3C2=O)C(=O)N1] |
| II-63 | 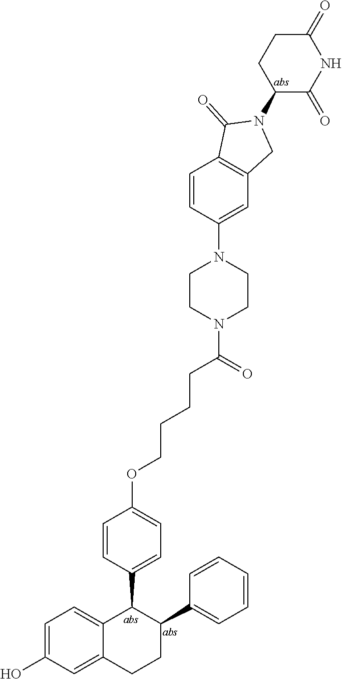 [SMILES: O=C1CC[C@H](N2Cc3cc(N4CCN(C(=O)CCCCOc5ccc([C@@H]6c7ccc(O)cc7CC[C@@H]6c6ccccc6)cc5)CC4)ccc3C2=O)C(=O)N1] |
| II-64 | 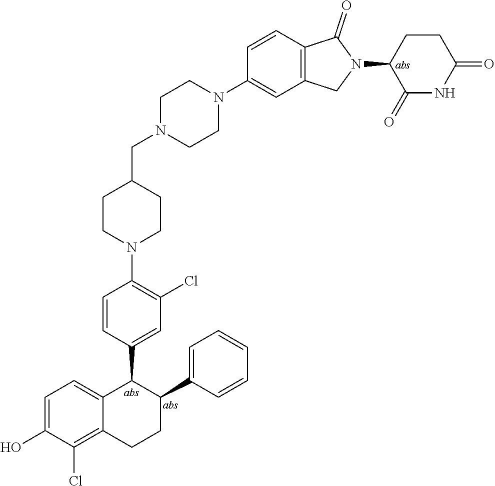 [SMILES: O=C1CC[C@H](N2Cc3cc(N4CCN(CC5CCN(c6ccc([C@@H]7c8ccc(O)c(Cl)c8CC[C@@H]7c7ccccc7)cc6Cl)CC5)CC4)ccc3C2=O)C(=O)N1] |
| II-65 | 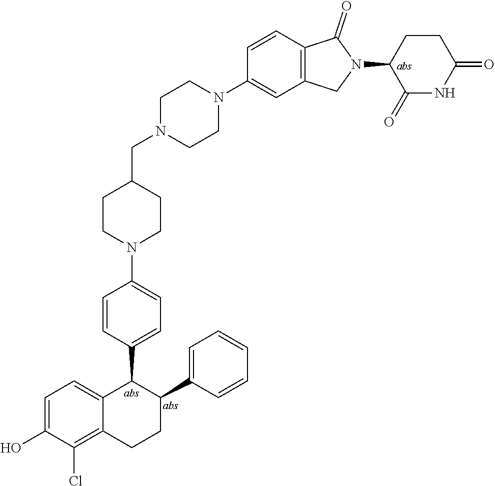 [SMILES: O=C1CC[C@H](N2Cc3cc(N4CCN(CC5CCN(c6ccc([C@@H]7c8ccc(O)c(Cl)c8CC[C@@H]7c7ccccc7)cc6)CC5)CC4)ccc3C2=O)C(=O)N1] |
| II-66 | 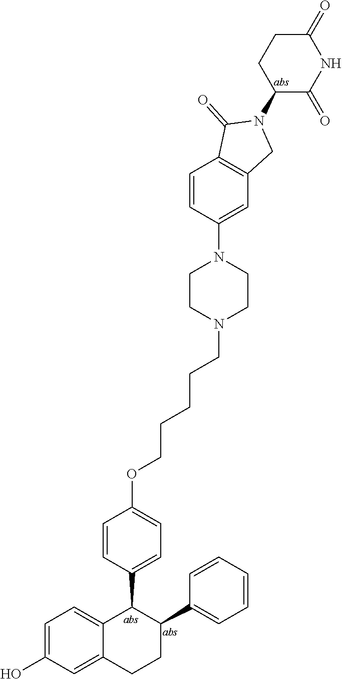 [SMILES: O=C1CC[C@H](N2Cc3cc(N4CCN(CCCCCOc5ccc([C@@H]6c7ccc(O)cc7CC[C@@H]6c6ccccc6)cc5)CC4)ccc3C2=O)C(=O)N1] |
| II-67 | 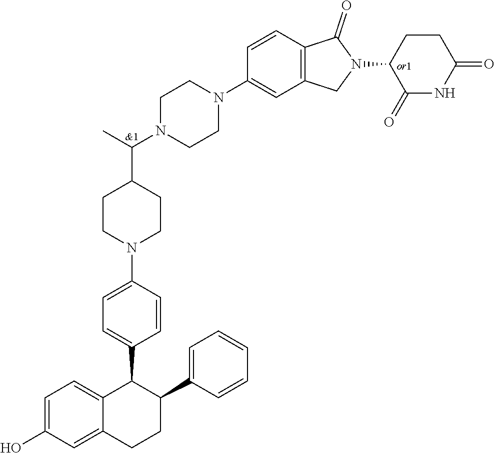 [SMILES: CC(C1CCN(c2ccc([C@@H]3c4ccc(O)cc4CC[C@@H]3c3ccccc3)cc2)CC1)N1CCN(c2ccc3c(c2)CN([C@@H]2CCC(=O)NC2=O)C3=O)CC1] |
| II-68 | 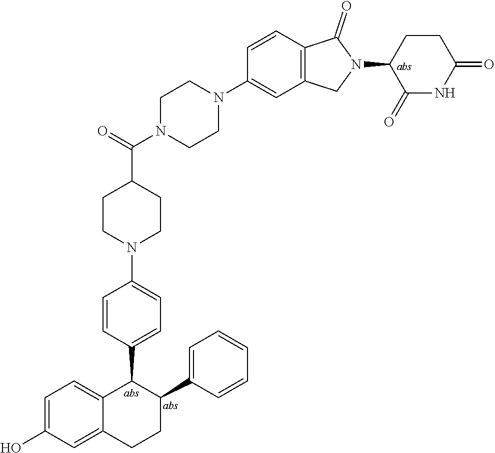 [SMILES: O=C1CC[C@H](N2Cc3cc(N4CCN(C(=O)C5CCN(c6ccc([C@@H]7c8ccc(O)cc8CC[C@@H]7c7ccccc7)cc6)CC5)CC4)ccc3C2=O)C(=O)N1] |
| II-69 | 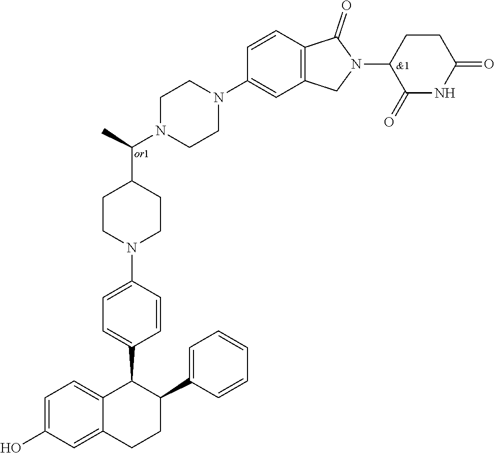 [SMILES: C[C@H](C1CCN(c2ccc([C@@H]3c4ccc(O)cc4CC[C@@H]3c3ccccc3)cc2)CC1)N1CCN(c2ccc3c(c2)CN(C2CCC(=O)NC2=O)C3=O)CC1] |
| II-70 | 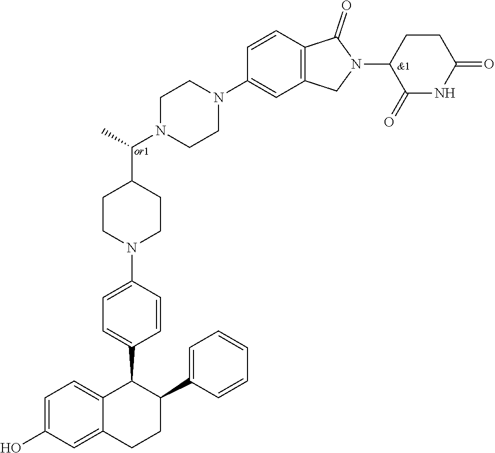 [SMILES: C[C@@H](C1CCN(c2ccc([C@@H]3c4ccc(O)cc4CC[C@@H]3c3ccccc3)cc2)CC1)N1CCN(c2ccc3c(c2)CN(C2CCC(=O)NC2=O)C3=O)CC1] |
| II-71 | 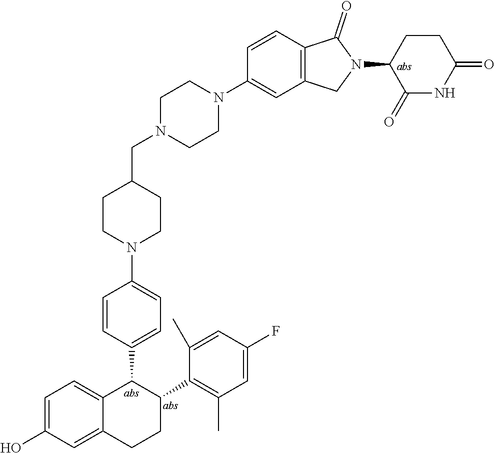 [SMILES: Cc1cc(F)cc(C)c1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| II-72 | 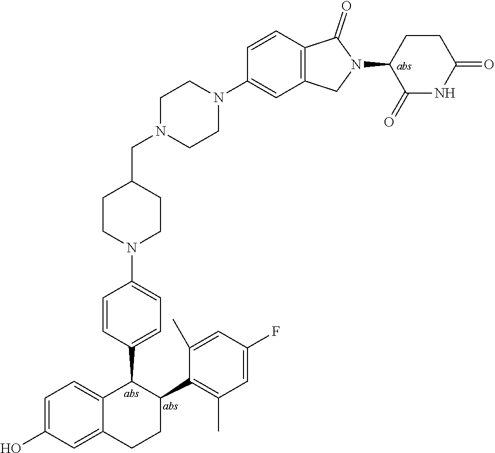 [SMILES: Cc1cc(F)cc(C)c1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| II-73 | 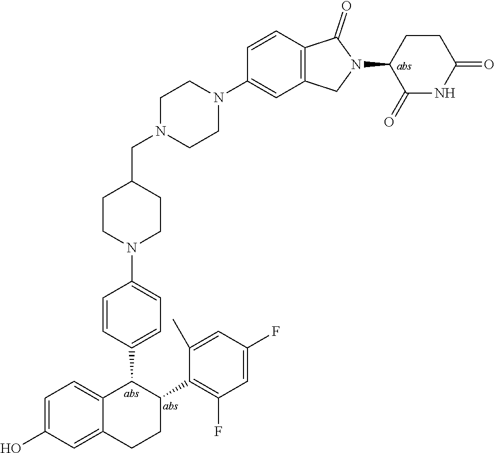 [SMILES: Cc1cc(F)cc(F)c1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] |
| II-74 | 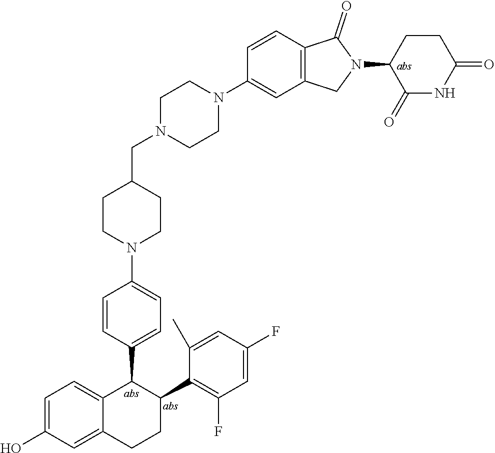 [SMILES: Cc1cc(F)cc(F)c1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] |
| II-75 | 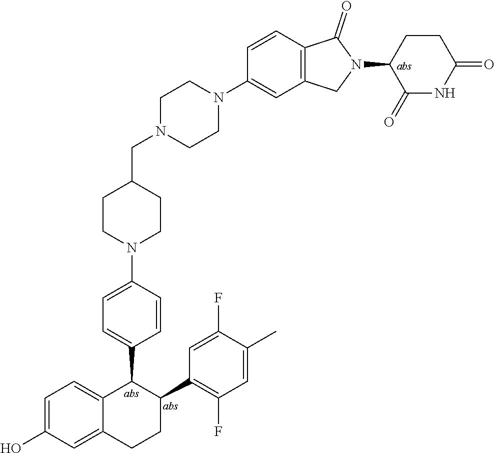 [SMILES: Cc1cc(F)c([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F] |
| II-76 | 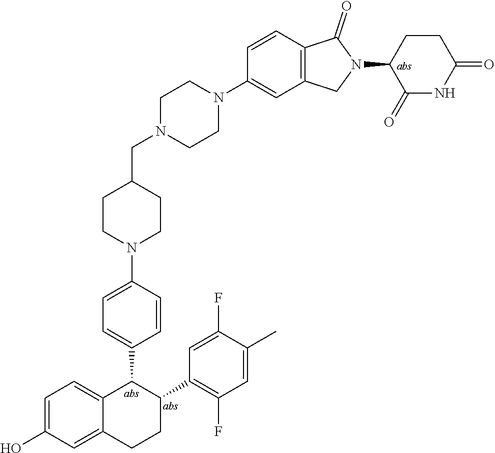 [SMILES: Cc1cc(F)c([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] |
| II-77 | 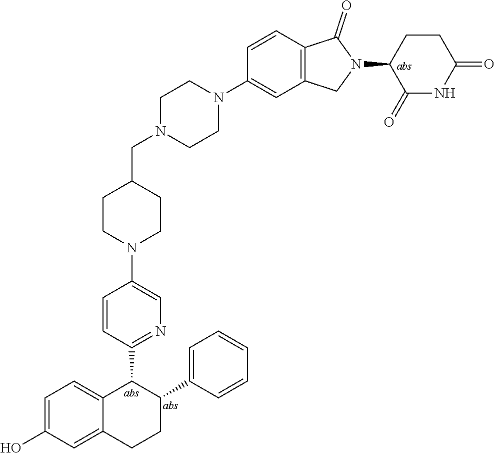 [SMILES: O=C1CC[C@H](N2Cc3cc(N4CCN(CC5CCN(c6ccc([C@H]7c8ccc(O)cc8CC[C@H]7c7ccccc7)nc6)CC5)CC4)ccc3C2=O)C(=O)N1] |
| II-78 | 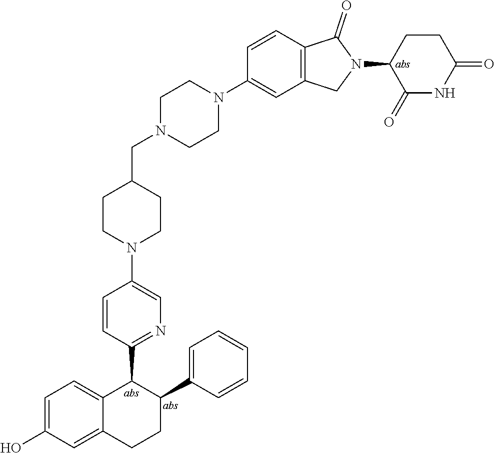 [SMILES: O=C1CC[C@H](N2Cc3cc(N4CCN(CC5CCN(c6ccc([C@@H]7c8ccc(O)cc8CC[C@@H]7c7ccccc7)nc6)CC5)CC4)ccc3C2=O)C(=O)N1] |
| II-79 | 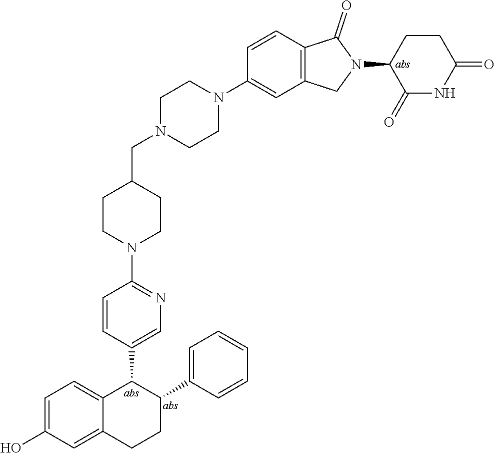 [SMILES: O=C1CC[C@H](N2Cc3cc(N4CCN(CC5CCN(c6ccc([C@H]7c8ccc(O)cc8CC[C@H]7c7ccccc7)cn6)CC5)CC4)ccc3C2=O)C(=O)N1] |
| II-80 | 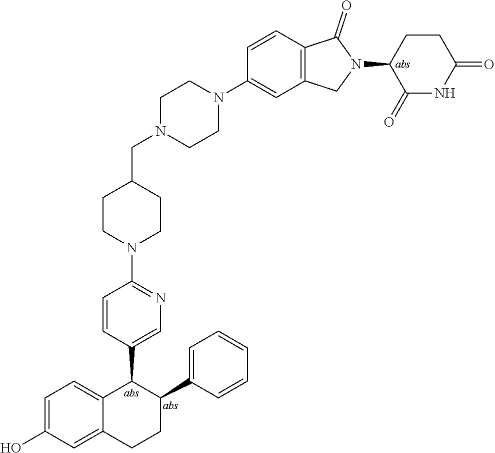 [SMILES: O=C1CC[C@H](N2Cc3cc(N4CCN(CC5CCN(c6ccc([C@@H]7c8ccc(O)cc8CC[C@@H]7c7ccccc7)cn6)CC5)CC4)ccc3C2=O)C(=O)N1] |
| II-81 | 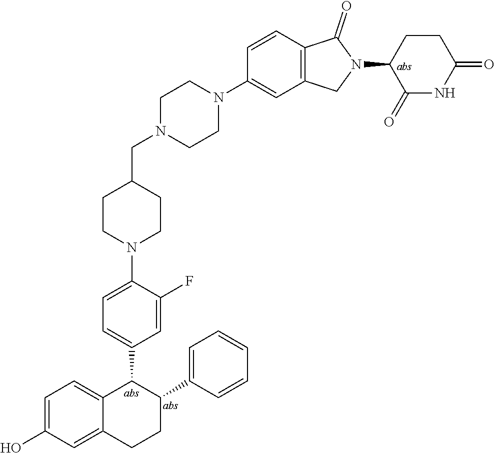 [SMILES: O=C1CC[C@H](N2Cc3cc(N4CCN(CC5CCN(c6ccc([C@H]7c8ccc(O)cc8CC[C@H]7c7ccccc7)cc6F)CC5)CC4)ccc3C2=O)C(=O)N1] |
| II-82 | 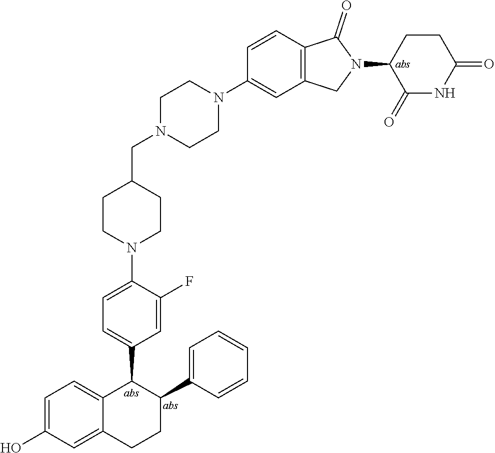 [SMILES: O=C1CC[C@H](N2Cc3cc(N4CCN(CC5CCN(c6ccc([C@@H]7c8ccc(O)cc8CC[C@@H]7c7ccccc7)cc6F)CC5)CC4)ccc3C2=O)C(=O)N1] |
| II-83 | 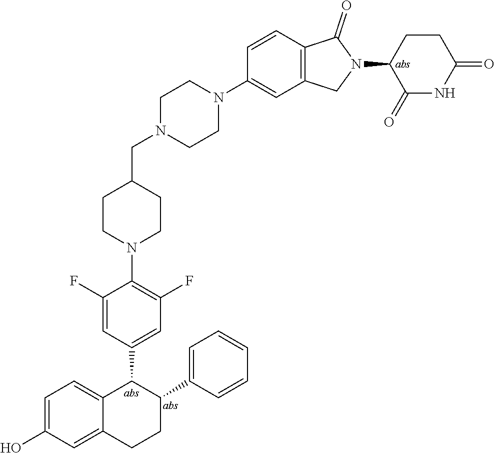 [SMILES: O=C1CC[C@H](N2Cc3cc(N4CCN(CC5CCN(c6c(F)cc([C@H]7c8ccc(O)cc8CC[C@H]7c7ccccc7)cc6F)CC5)CC4)ccc3C2=O)C(=O)N1] |
| II-84 | 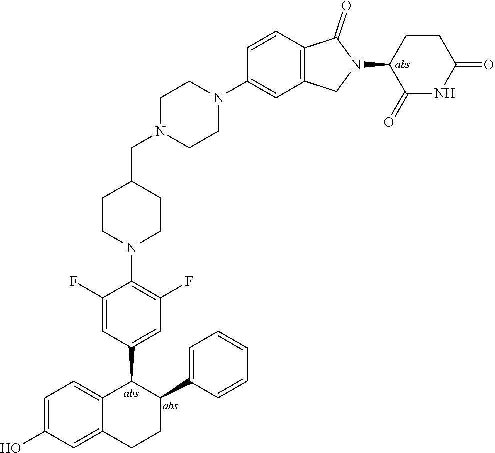 [SMILES: O=C1CC[C@H](N2Cc3cc(N4CCN(CC5CCN(c6c(F)cc([C@@H]7c8ccc(O)cc8CC[C@@H]7c7ccccc7)cc6F)CC5)CC4)ccc3C2=O)C(=O)N1] |
| II-85 | 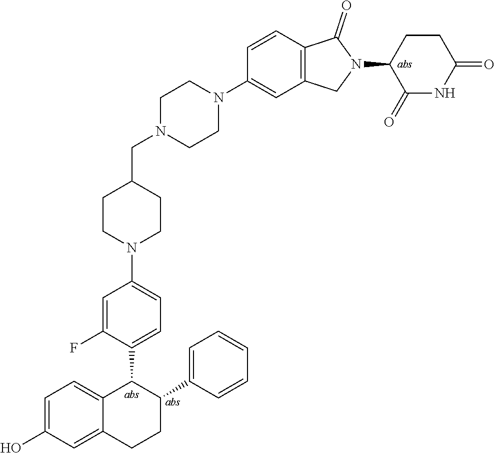 [SMILES: O=C1CC[C@H](N2Cc3cc(N4CCN(CC5CCN(c6ccc([C@H]7c8ccc(O)cc8CC[C@H]7c7ccccc7)c(F)c6)CC5)CC4)ccc3C2=O)C(=O)N1] |
| II-86 | 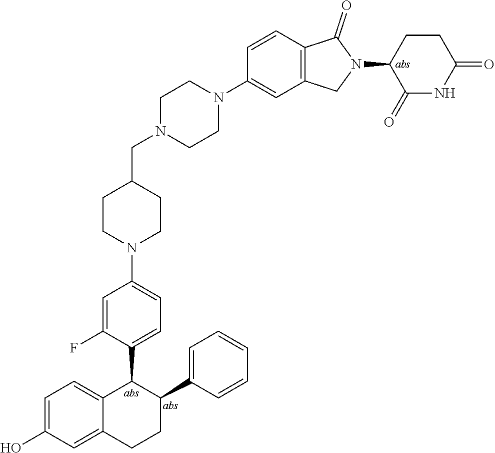 [SMILES: O=C1CC[C@H](N2Cc3cc(N4CCN(CC5CCN(c6ccc([C@@H]7c8ccc(O)cc8CC[C@@H]7c7ccccc7)c(F)c6)CC5)CC4)ccc3C2=O)C(=O)N1] |
| II-88 | 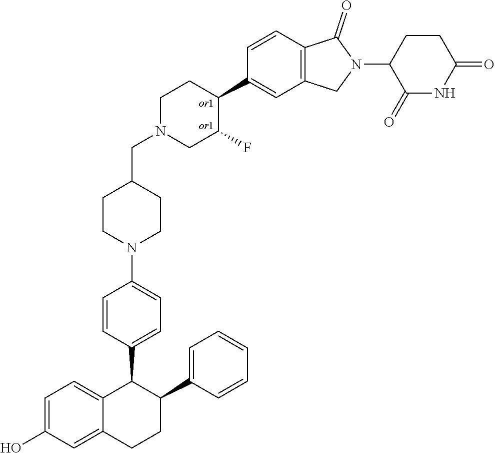 [SMILES: O=C1CCC(N2Cc3cc([C@@H]4CCN(CC5CCN(c6ccc([C@@H]7c8ccc(O)cc8CC[C@@H]7c7ccccc7)cc6)CC5)C[C@H]4F)ccc3C2=O)C(=O)N1] |
| II-89 | 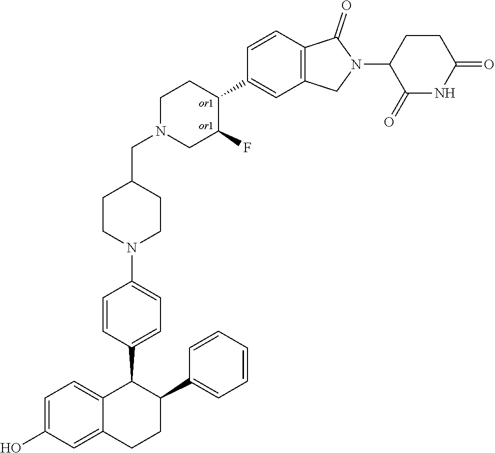 [SMILES: O=C1CCC(N2Cc3cc([C@H]4CCN(CC5CCN(c6ccc([C@@H]7c8ccc(O)cc8CC[C@@H]7c7ccccc7)cc6)CC5)C[C@@H]4F)ccc3C2=O)C(=O)N1] |
| II-90 (*) | 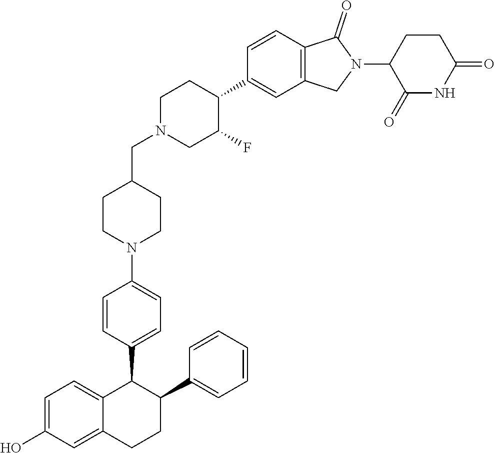 [SMILES: O=C1CCC(N2Cc3cc([C@H]4CCN(CC5CCN(c6ccc([C@@H]7c8ccc(O)cc8CC[C@@H]7c7ccccc7)cc6)CC5)C[C@H]4F)ccc3C2=O)C(=O)N1] |
| II-91 | 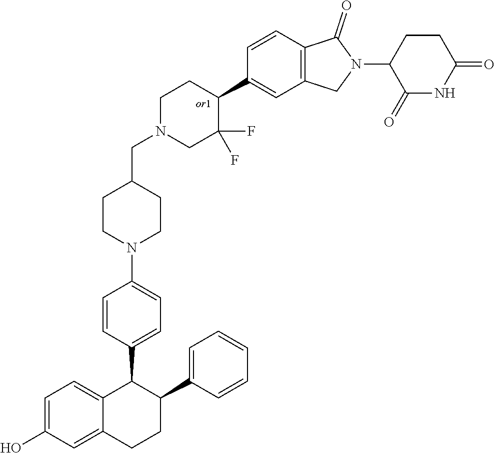 [SMILES: O=C1CCC(N2Cc3cc([C@@H]4CCN(CC5CCN(c6ccc([C@@H]7c8ccc(O)cc8CC[C@@H]7c7ccccc7)cc6)CC5)CC4(F)F)ccc3C2=O)C(=O)N1] |
| II-92 | 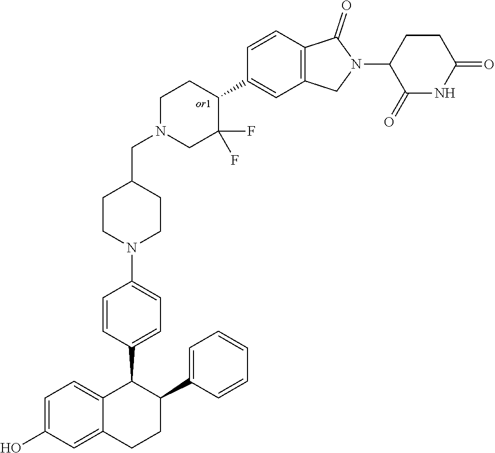 [SMILES: O=C1CCC(N2Cc3cc([C@H]4CCN(CC5CCN(c6ccc([C@@H]7c8ccc(O)cc8CC[C@@H]7c7ccccc7)cc6)CC5)CC4(F)F)ccc3C2=O)C(=O)N1] |
| II-93 (*) | 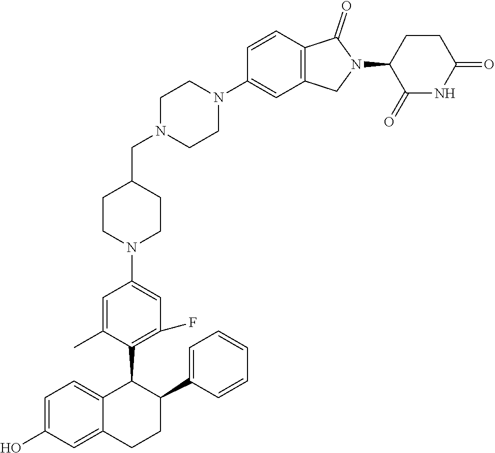 [SMILES: Cc1cc(N2CCC(CN3CCN(c4ccc5c(c4)CN([C@H]4CCC(=O)NC4=O)C5=O)CC3)CC2)cc(F)c1[C@@H]1c2ccc(O)cc2CC[C@@H]1c1ccccc1] |
| II-94 | 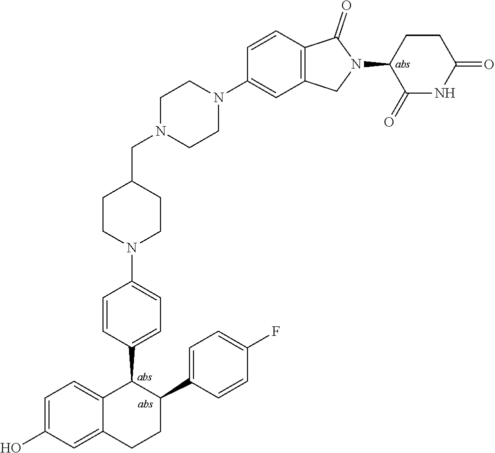 [SMILES: O=C1CC[C@H](N2Cc3cc(N4CCN(CC5CCN(c6ccc([C@@H]7c8ccc(O)cc8CC[C@@H]7c7ccc(F)cc7)cc6)CC5)CC4)ccc3C2=O)C(=O)N1] |
| II-95 | 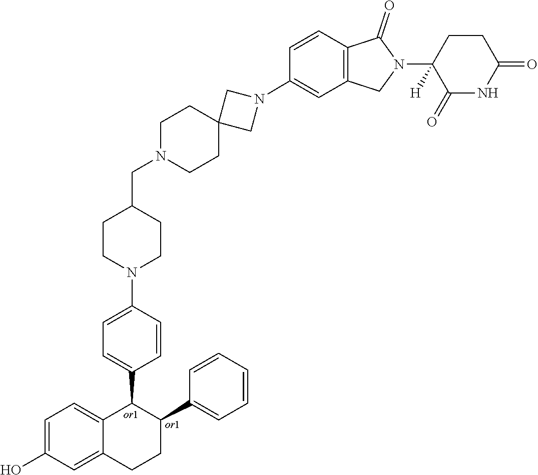 [SMILES: O=C1CC[C@H](N2Cc3cc(N4CC5(CCN(CC6CCN(c7ccc([C@@H]8c9ccc(O)cc9CC[C@@H]8c8ccccc8)cc7)CC6)CC5)C4)ccc3C2=O)C(=O)N1] |
| II-96 | 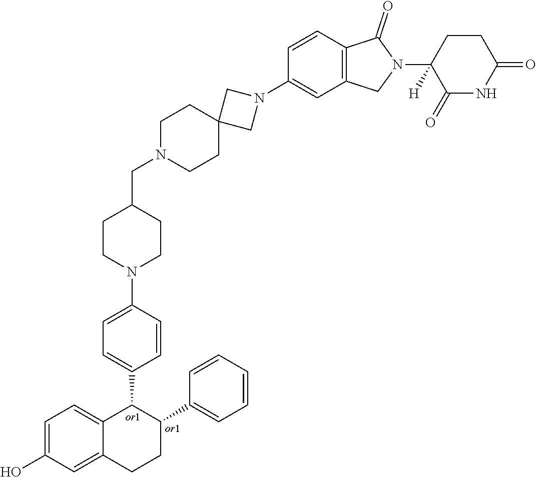 [SMILES: O=C1CC[C@H](N2Cc3cc(N4CC5(CCN(CC6CCN(c7ccc([C@H]8c9ccc(O)cc9CC[C@H]8c8ccccc8)cc7)CC6)CC5)C4)ccc3C2=O)C(=O)N1] |
| II-97 | 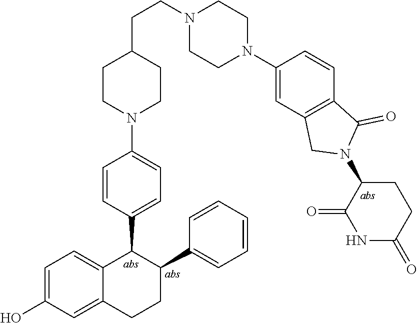 [SMILES: O=C1CC[C@H](N2Cc3cc(N4CCN(CCC5CCN(c6ccc([C@@H]7c8ccc(O)cc8CC[C@@H]7c7ccccc7)cc6)CC5)CC4)ccc3C2=O)C(=O)N1] |
| II-98 | 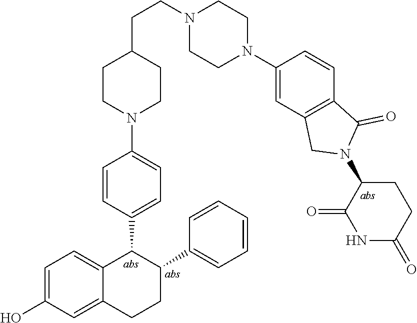 [SMILES: O=C1CC[C@H](N2Cc3cc(N4CCN(CCC5CCN(c6ccc([C@H]7c8ccc(O)cc8CC[C@H]7c7ccccc7)cc6)CC5)CC4)ccc3C2=O)C(=O)N1] |
| II-99 | 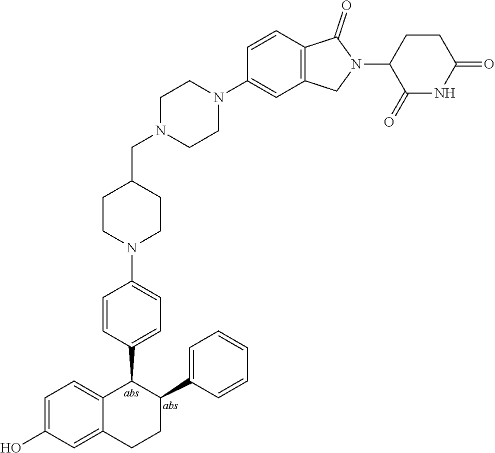 [SMILES: O=C1CCC(N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] |
| II-100 | 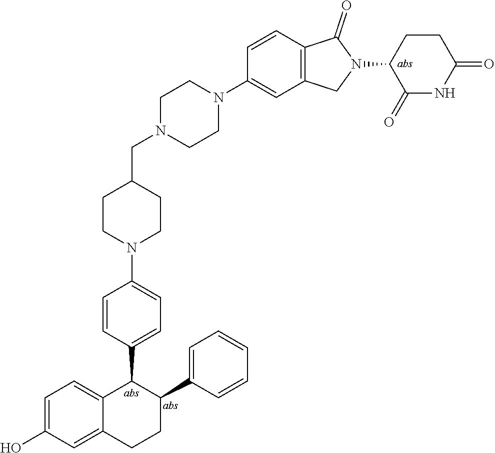 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] |
| II-101 | 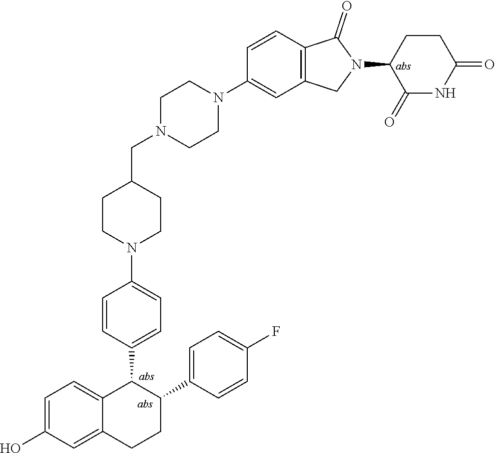 [SMILES: O=C1CC[C@H](N2Cc3cc(N4CCN(CC5CCN(c6ccc([C@H]7c8ccc(O)cc8CC[C@H]7c7ccc(F)cc7)cc6)CC5)CC4)ccc3C2=O)C(=O)N1] |
| II-102 | 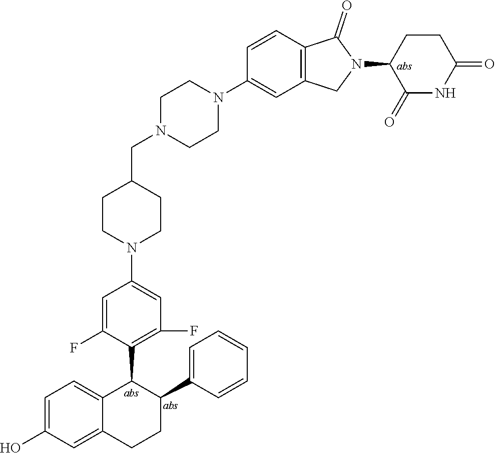 [SMILES: O=C1CC[C@H](N2Cc3cc(N4CCN(CC5CCN(c6cc(F)c([C@@H]7c8ccc(O)cc8CC[C@@H]7c7ccccc7)c(F)c6)CC5)CC4)ccc3C2=O)C(=O)N1] |
| II-103 | 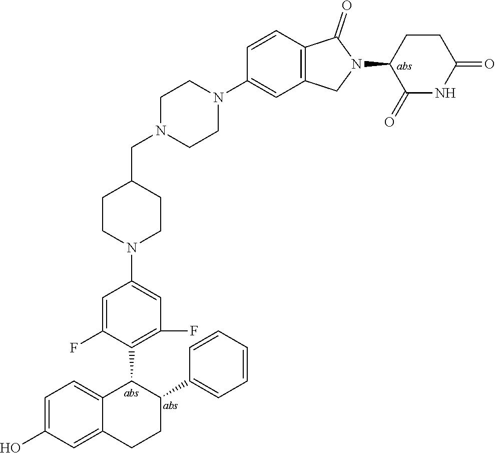 [SMILES: O=C1CC[C@H](N2Cc3cc(N4CCN(CC5CCN(c6cc(F)c([C@H]7c8ccc(O)cc8CC[C@H]7c7ccccc7)c(F)c6)CC5)CC4)ccc3C2=O)C(=O)N1] |
| II-104 | 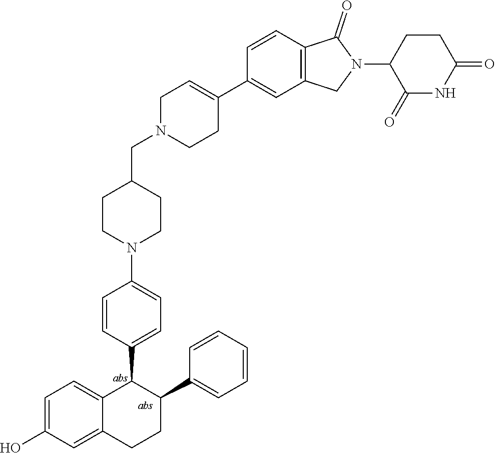 [SMILES: O=C1CCC(N2Cc3cc(C4=CCN(CC5CCN(c6ccc([C@@H]7c8ccc(O)cc8CC[C@@H]7c7ccccc7)cc6)CC5)CC4)ccc3C2=O)C(=O)N1] |
| II-105 | 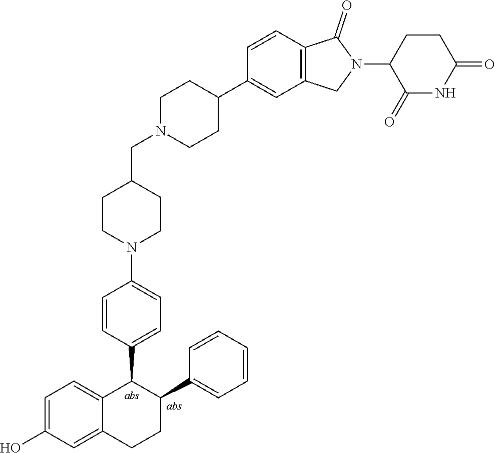 [SMILES: O=C1CCC(N2Cc3cc(C4CCN(CC5CCN(c6ccc([C@@H]7c8ccc(O)cc8CC[C@@H]7c7ccccc7)cc6)CC5)CC4)ccc3C2=O)C(=O)N1] |
| II-106 | 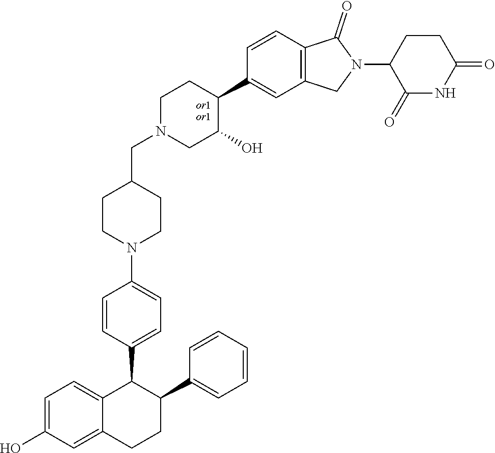 [SMILES: O=C1CCC(N2Cc3cc([C@@H]4CCN(CC5CCN(c6ccc([C@@H]7c8ccc(O)cc8CC[C@@H]7c7ccccc7)cc6)CC5)C[C@H]4O)ccc3C2=O)C(=O)N1] |
| II-107 | 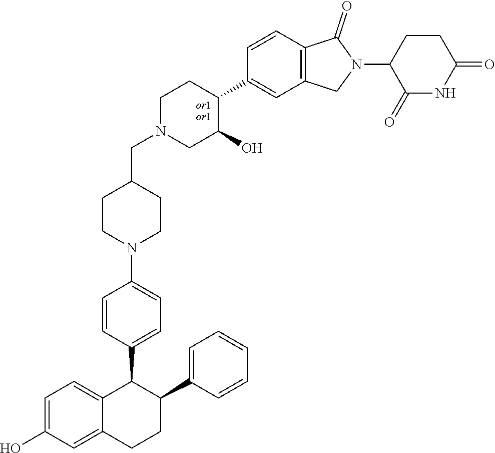 [SMILES: O=C1CCC(N2Cc3cc([C@H]4CCN(CC5CCN(c6ccc([C@@H]7c8ccc(O)cc8CC[C@@H]7c7ccccc7)cc6)CC5)C[C@@H]4O)ccc3C2=O)C(=O)N1] |
| II-108 | 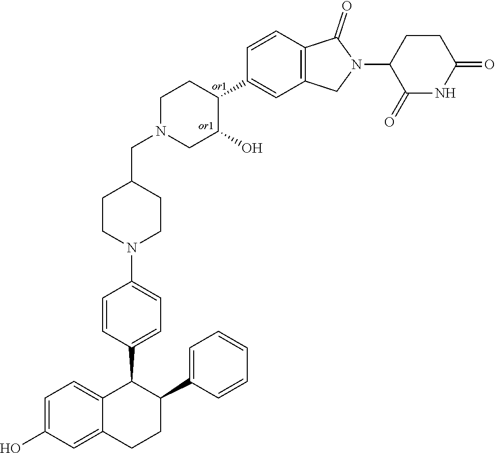 [SMILES: O=C1CCC(N2Cc3cc([C@H]4CCN(CC5CCN(c6ccc([C@@H]7c8ccc(O)cc8CC[C@@H]7c7ccccc7)cc6)CC5)C[C@H]4O)ccc3C2=O)C(=O)N1] |
| II-109 | 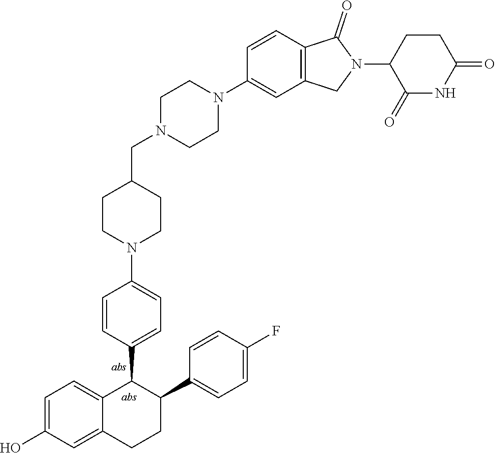 [SMILES: O=C1CCC(N2Cc3cc(N4CCN(CC5CCN(c6ccc([C@@H]7c8ccc(O)cc8CC[C@@H]7c7ccc(F)cc7)cc6)CC5)CC4)ccc3C2=O)C(=O)N1] |
| II-110 | 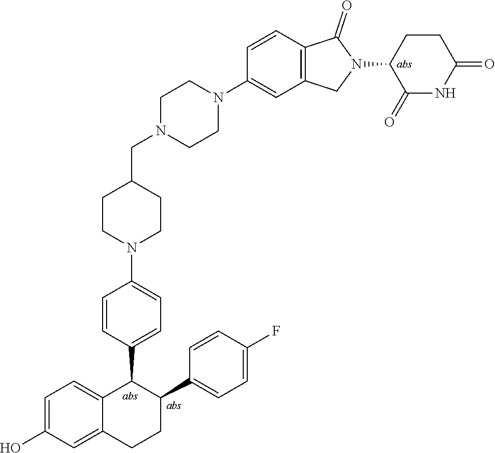 [SMILES: O=C1CC[C@@H](N2Cc3cc(N4CCN(CC5CCN(c6ccc([C@@H]7c8ccc(O)cc8CC[C@@H]7c7ccc(F)cc7)cc6)CC5)CC4)ccc3C2=O)C(=O)N1] |
| II-126‡ | 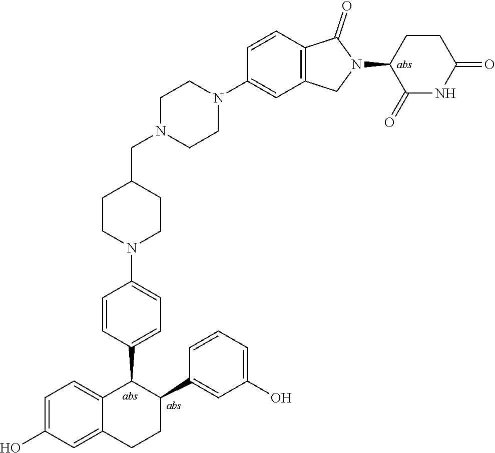 [SMILES: O=C1CC[C@H](N2Cc3cc(N4CCN(CC5CCN(c6ccc([C@@H]7c8ccc(O)cc8CC[C@@H]7c7cccc(O)c7)cc6)CC5)CC4)ccc3C2=O)C(=O)N1] |
| II-127‡ | 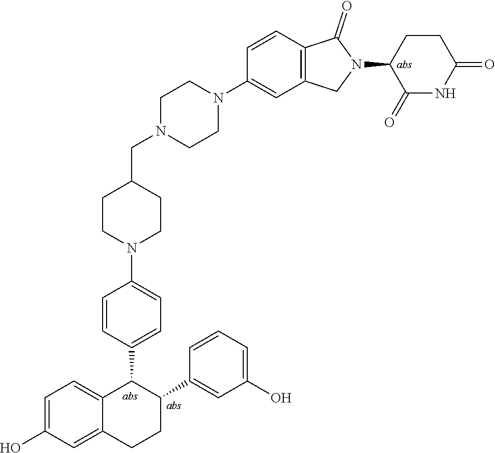 [SMILES: O=C1CC[C@H](N2Cc3cc(N4CCN(CC5CCN(c6ccc([C@H]7c8ccc(O)cc8CC[C@H]7c7cccc(O)c7)cc6)CC5)CC4)ccc3C2=O)C(=O)N1] |
| II-130 | 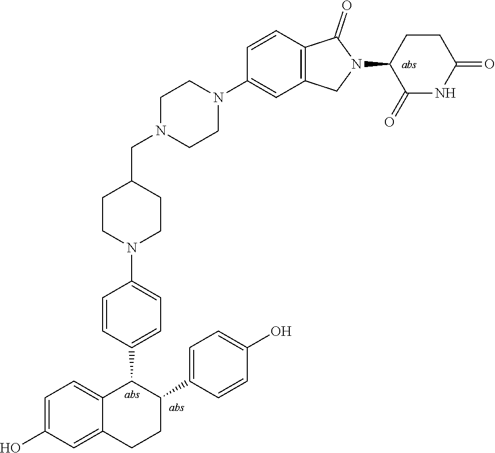 [SMILES: O=C1CC[C@H](N2Cc3cc(N4CCN(CC5CCN(c6ccc([C@H]7c8ccc(O)cc8CC[C@H]7c7ccc(O)cc7)cc6)CC5)CC4)ccc3C2=O)C(=O)N1] |
| II-131 | 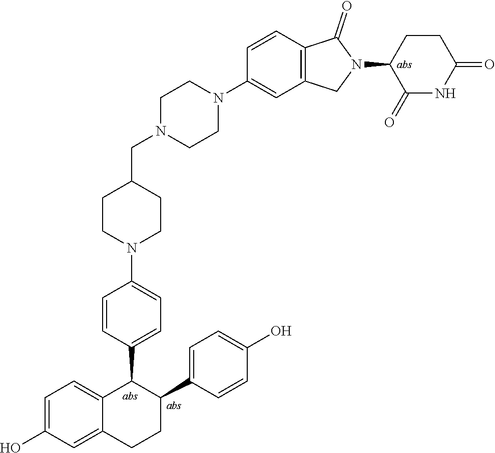 [SMILES: O=C1CC[C@H](N2Cc3cc(N4CCN(CC5CCN(c6ccc([C@@H]7c8ccc(O)cc8CC[C@@H]7c7ccc(O)cc7)cc6)CC5)CC4)ccc3C2=O)C(=O)N1] |
| II-343 | 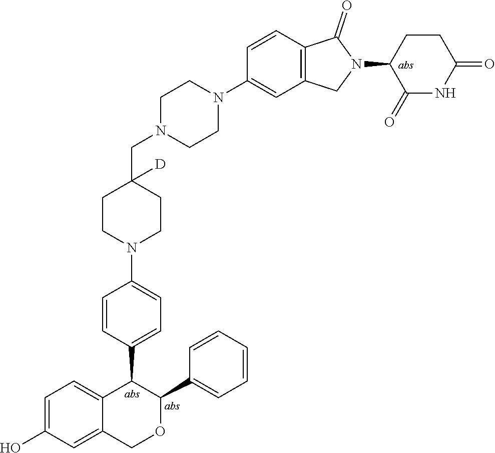 [SMILES: [2H]C1(CN2CCN(c3ccc4c(c3)CN([C@H]3CCC(=O)NC3=O)C4=O)CC2)CCN(c2ccc([C@@H]3c4ccc(O)cc4CO[C@@H]3c3ccccc3)cc2)CC1] |

“‡” denotes compounds for which the initial stereochemistry was arbitrarily assigned, as indicated by the previous “or1” designation at the respective stereocenters. Current stereochemistry assignment has been confirmed by independent experiments, as indicated by the “abs” designation at the respective stereocenters.

In chemical structures in Table 1 or Table 2, above, and the Examples, below, stereogenic centers are described according to the Enhanced Stereo Representation format (MDL/Biovia, e.g. using labels “or1”, “or2”, “abs”, “and1”).

In some embodiments, the present disclosure comprises a compound of formula I-1, I-1′, I-2, I-2′, I-3 or I-3′ selected from those depicted in Table 1 or Table 2, above, or a pharmaceutically acceptable salt, stereoisomer, or mixture of stereoisomers thereof. In some embodiments, the present disclosure comprises a compound of formula I-1 selected from those depicted in Table 1 or Table 2, above, or a pharmaceutically acceptable salt, diastereomer, or mixture of diastereomers thereof. In some embodiments, the present disclosure provides a compound of formula I-1, I-1′, I-2, I-2′, I-3 or I-3′ selected from those depicted in Table 1 or Table 2, above, or a pharmaceutically acceptable salt thereof. In some embodiments, the present disclosure provides a compound of formula I-1, I-1′, I-2, I-2′, I-3 or I-3′ selected from those depicted in Table 1 or Table 2, above.

The compounds in Table 1 or Table 2 were made in accordance with chemical procedures described in the Examples section, or similar procedures that would be readily understood by a person of skill in the art on the basis of the instant disclosure and knowledge present in the art. The compounds marked with (*) in Table 1 or Table 2 are prophetic examples and can be made by methods similar to those included in the Examples section.

### 4. Uses, Formulation, and Administration

### Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that is effective to measurably degrade ERα, or a mutant thereof, in a biological sample or in a patient. In certain embodiments, the amount of compound in compositions of this disclosure is such that it is effective to measurably degrade ERα, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “degratorily active metabolite or residue thereof” means that a metabolite or residue thereof is also a degrader of ERα, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceuties, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added.

Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the degradation of ERα. In some embodiments, the nuclear transcription factor degraded by the compounds and compositions described herein is ERα.

Compounds or compositions of the disclosure can be useful in applications that benefit from the degradation of ERα. For example, ERα degraders of the present disclosure are useful for the treatment of cellular proliferative diseases generally.

The activity of a compound utilized in this disclosure as an degrader of ERα, or a mutant thereof, may be assayed in vitro, in vivo or in a cell line. In vitro assays include assays that determine inhibition of either the signaling activity and/or the subsequent functional consequences, of an activated ERα, or a mutant thereof. Alternative in vitro assays quantitate the ability of the degrader to degrade ERα. Representative in vitro and in vivo assays useful in assaying an ERα degrader include those described and disclosed in the patent and scientific publications described herein. Detailed conditions for assaying a compound utilized in this disclosure as an degrader of ERα, or a mutant thereof, are set forth in the Examples below.

### Treatment of Disorders

Provided compounds are degraders of ERα and are therefore useful for treating one or more disorders associated with activity of ERα or mutants thereof. Thus, in certain embodiments, the present disclosure provides a method of treating an ERα-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating an ERα-mediated disorder in a subject comprising administering a therapeutically effective amount of a compound of the present disclosure, or a pharmaceutically acceptable composition thereof, to a subject in need thereof. In some embodiments, the subject has a mutant ERα. In some embodiments, the subject has ERα containing a D538G, Y537S, or L536R mutation.

As used herein, the term “ERα-mediated” disorders, diseases, and/or conditions means any disease or other deleterious condition in which ERα or a mutant thereof is known to play a role. Accordingly, another embodiment of the present disclosure relates to treating or lessening the severity of one or more diseases in which ERα, or a mutant thereof, is known to play a role. Such ERα-mediated disorders include, but are not limited to, cellular proliferative disorders (e.g. cancer). In some embodiments, the ERα-mediated disorder is a disorder mediated by a mutant ERα. In some embodiments, the ERα-mediated disorder is a disorder mediated by an ERα containing a D538G, Y537S, or L536R mutation.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof; and (iii) administering said provided compound in a therapeutically effective amount to treat, suppress and/or prevent the disease state or condition in a subject in need of such treatment. In some embodiments, the subject has a mutant ERα. In some embodiments, the subject has ERα containing a D538G, Y537S, or L536R mutation.

In some embodiments, the method of treatment comprises the steps of: i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment. In some embodiments, the subject has a mutant ERα. In some embodiments, the subject has ERα containing a D538G, Y537S, or L536R mutation.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a hematopoietic cancer. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+ breast cancer. In some embodiments, the cancer is ER+/HER2-breast cancer. In some embodiments, the cancer is ER+/HER2-breast cancer, and the subject is intolerant to, or ineligible for, treatment with alpelisib. In some embodiments, the cancer is sporadic breast cancer. In some embodiments, the cancer is Cowden disease. In some embodiments, the cancer is ER+ advanced/metastatic breast cancer. In some embodiments, the cancer is ER+/HER2-advanced/metastatic breast cancer. In some embodiments, the breast cancer has mutant ERα. In some embodiments, the breast cancer has ERα containing a D538G, Y537S, or L536R mutation.

In some embodiments, the cancer is uterine cancer. In some embodiments, the uterine cancer has mutant ERα. In some embodiments, the uterine cancer has ERα containing a D538G, Y537S, or L536R mutation.

In some embodiments, the ERα-mediated disorder is endometriosis. In some embodiments, the endometriosis has mutant ERα. In some embodiments, the endometriosis has ERα containing a D538G, Y537S, or L536R mutation.

In some embodiments, the cellular proliferative disease displays overexpression or amplification of ERα, or somatic mutation of ERα.

### Routes of Administration and Dosage Forms

The compounds and compositions, according to the methods of the present disclosure, may be administered using any amount and any route of administration effective for treating or lessening the severity of the disorder (e.g. a proliferative disorder). The exact amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Degradation of ERα

According to one embodiment, the disclosure relates to a method of degrading ERα in a biological sample comprising the step of contacting said biological sample with a compound of this disclosure, or a composition comprising said compound. According to another embodiment, the disclosure relates to a method of degrading ERα, or a mutant thereof, in a biological sample comprising the step of contacting said biological sample with a compound of this disclosure, or a composition comprising said compound. According to another embodiment, the disclosure relates to a method of degrading ERα, or a mutant thereof, in a biological sample comprising the step of contacting said biological sample with a compound of this disclosure, or a composition comprising said compound. In some embodiments, the ERα is a mutant ERα. In some embodiments, ERα contains a D538G, Y537S, or L536R mutation.

Without wishing to be bound by any particular theory, it is contemplated that compounds of the present invention catalyze the destruction of ERα. The compounds of the present invention are proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of ERα to the E3 ligase will thus result in the destruction of the ERα protein. The variable chain of atoms can include, for example, rings, heteroatoms, and/or repeating polymeric units. It can be rigid or flexible. It can be attached to the two portions described above using standard techniques in the art of organic synthesis.

In some embodiments, the disclosure provides a method of selectively inhibiting a mutant ERα over a wild-type ERα.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a ERα (for example, ERα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting ERα-mediated transcription regulation in a patient comprising the step of administering to said patient a compound of the present disclosure, or a composition comprising said compound.

According to another embodiment, the disclosure relates to a method of inhibiting activity of a ERα, or a mutant thereof, in a patient comprising the step of administering to said patient a compound of the present disclosure, or a composition comprising said compound. In some embodiments, the disclosure relates to a method of inhibiting activity of ERα, or a mutant thereof, in a patient comprising the step of administering to said patient a compound of the present disclosure, or a composition comprising said compound. In some embodiments, the ERα is a mutant ER. In some embodiments, the ERα contains a D538G, Y537S, or L536R mutation.

According to another embodiment, the present disclosure provides a method for treating a disorder mediated by a ERα, or a mutant thereof, in a patient in need thereof, comprising the step of administering to said patient a compound according to the present disclosure or pharmaceutically acceptable composition thereof. In some embodiments, the present disclosure provides a method for treating a disorder mediated by ERα, or a mutant thereof, in a patient in need thereof, comprising the step of administering to said patient a compound according to the present disclosure or pharmaceutically acceptable composition thereof. In some embodiments, the ERα is a mutant ERα. In some embodiments, the ERα contains a D538G, Y537S, or L536R mutation.

According to another embodiment, the present disclosure provides a method of inhibiting ERα-mediated transcription regulation, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting ERα-mediated transcription regulation in a subject, comprising administering a therapeutically effective amount of a compound according to the present disclosure, or a pharmaceutically acceptable composition thereof, to a subject in need thereof. In some embodiments, the ERα is a mutant ERα. In some embodiments, the ERα contains a D538G, Y537S, or L536R mutation. In some embodiments, the subject has a D538G, Y537S, or L536R mutant ERα. In some embodiments, the subject has ERα containing D538G, Y537S, or L536R mutation.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

The structure of the active compounds identified by code numbers, generic or trade names may be taken from the actual edition of the standard compendium “The Merck Index” or from databases, e.g. Patents International (e.g. IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### Selected Embodiments

1. A compound of formula I-3′:

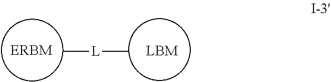

or a pharmaceutically acceptable salt thereof, wherein:
ERBM is selected from

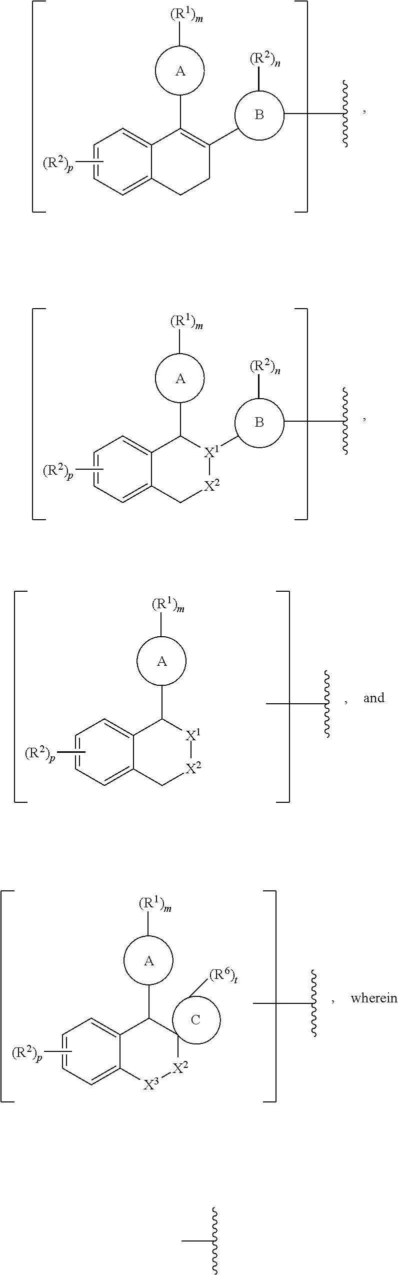

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A4} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are optionally taken together with their intervening atoms to having 0-3 heteroatoms, in addition to the nitrogen, independently selected from nitrogen, oxygen, and sulfur;
Ring A and Ring independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

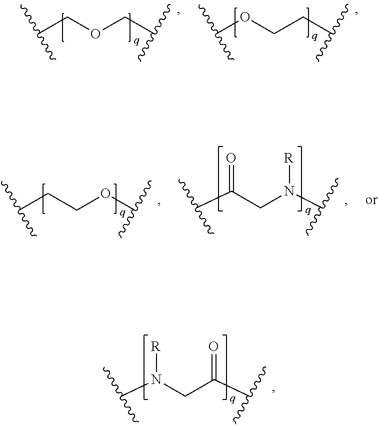

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

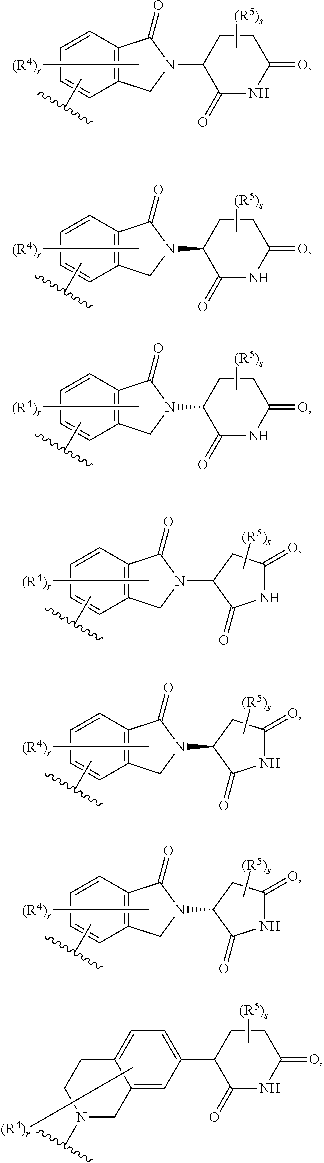

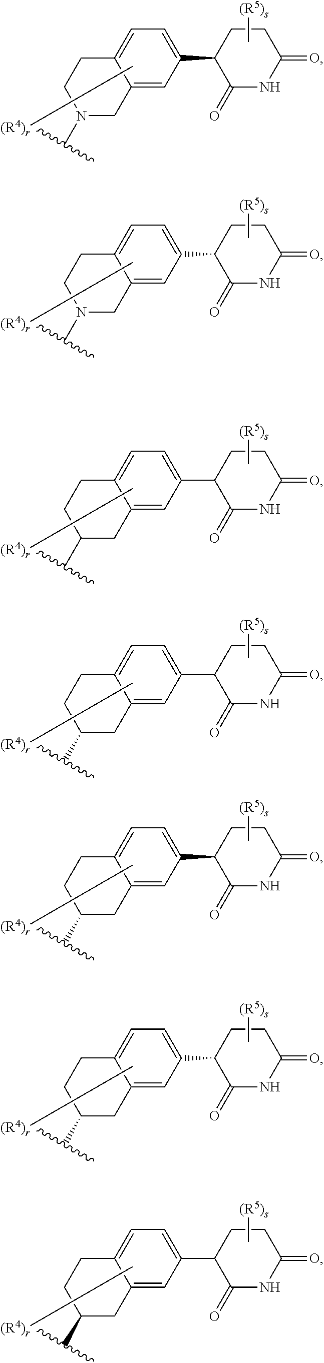

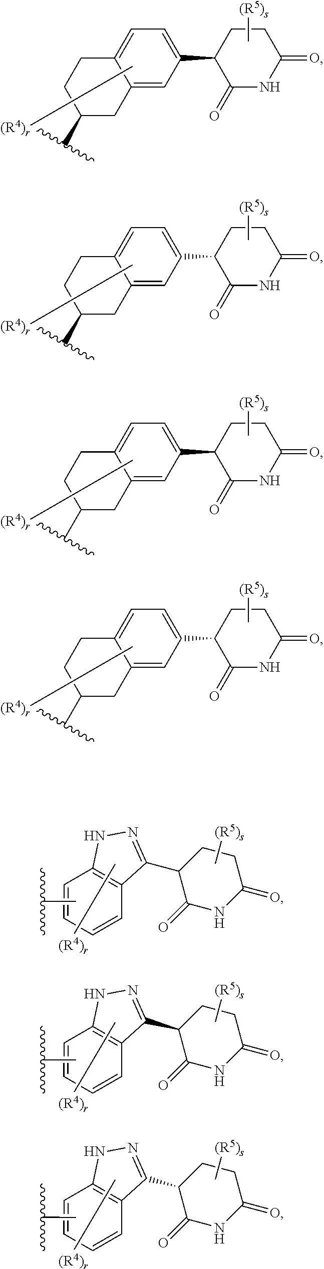

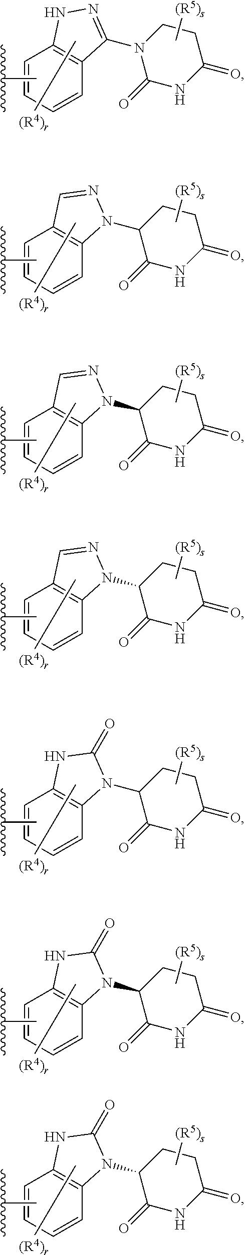

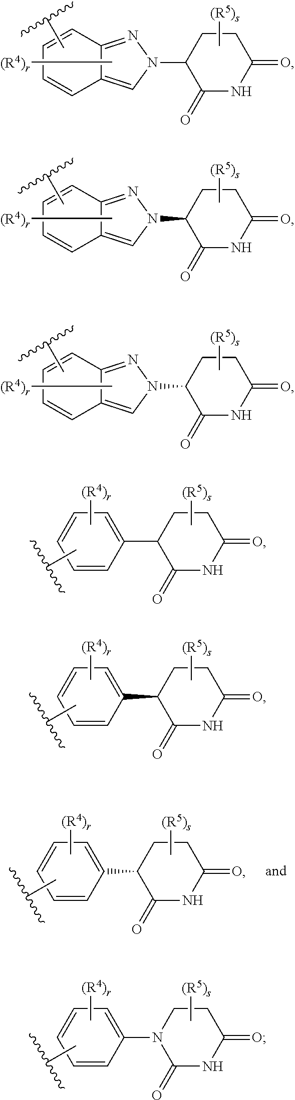

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

2. A compound of formula I-3′:

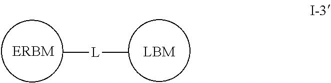

or a pharmaceutically acceptable salt thereof, wherein:
ERBM is selected from

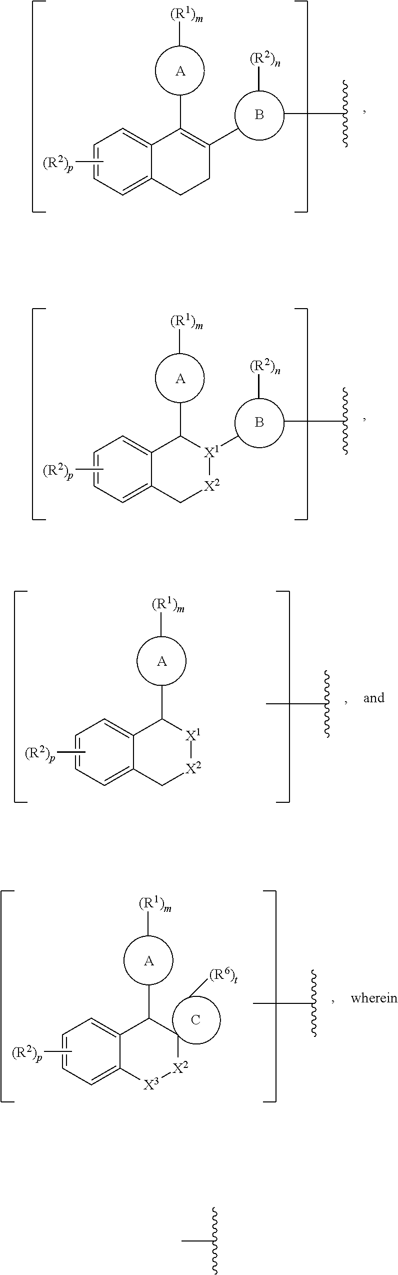

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R⁴⁵), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A5} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O) ¿R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

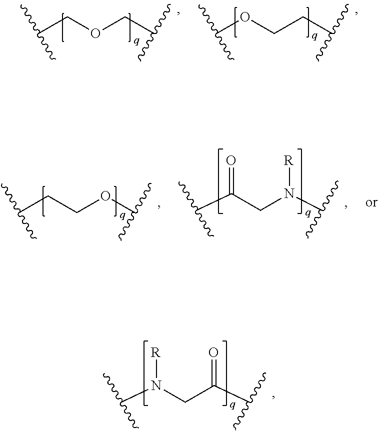

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

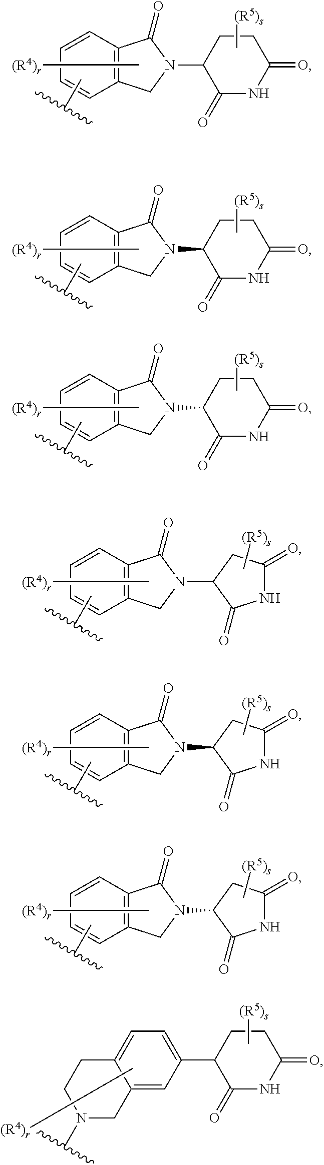

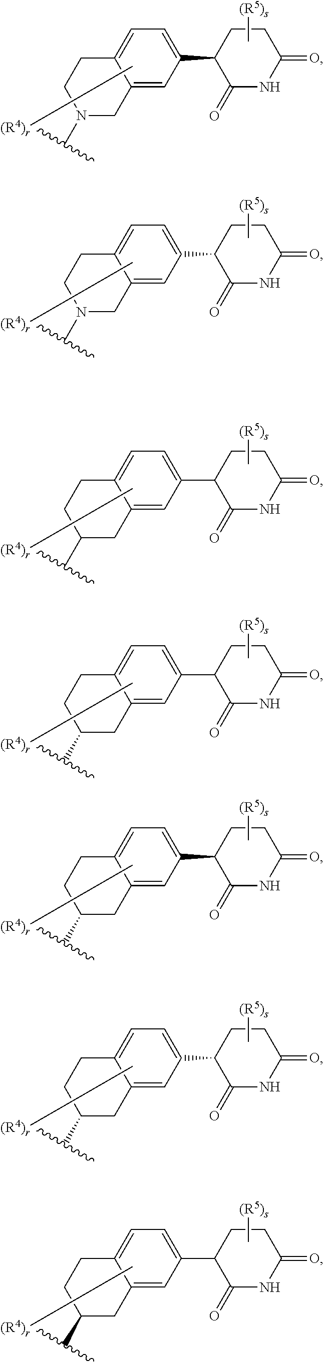

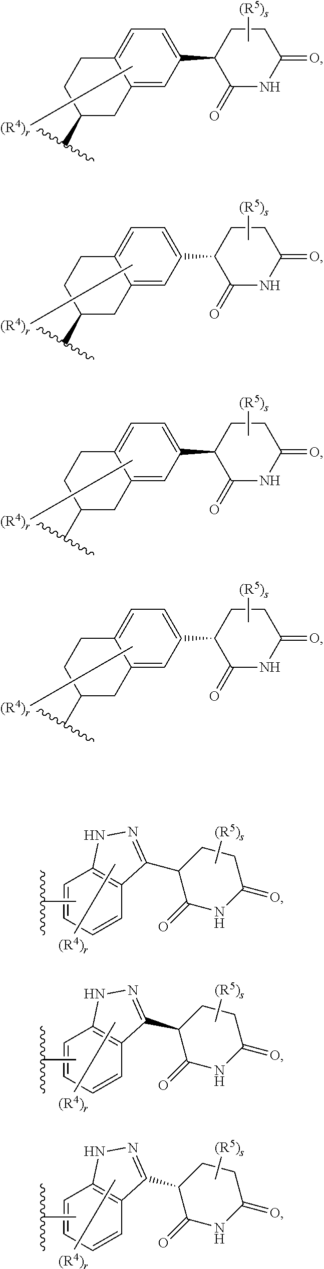

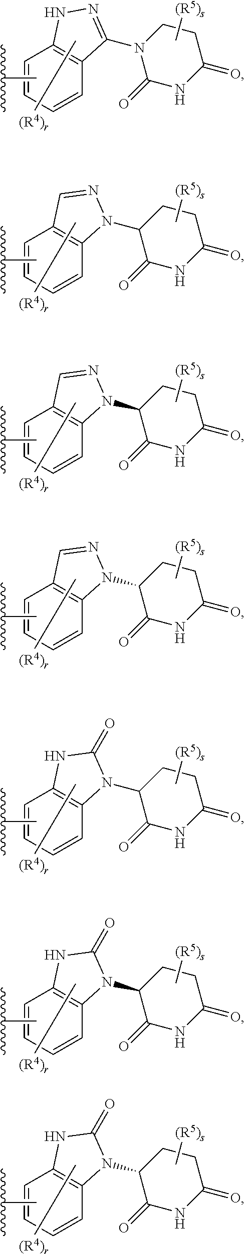

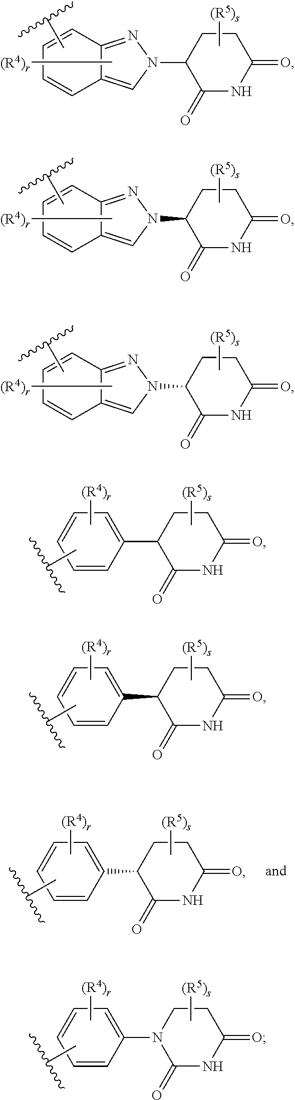

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4,
provided that when ERBM is

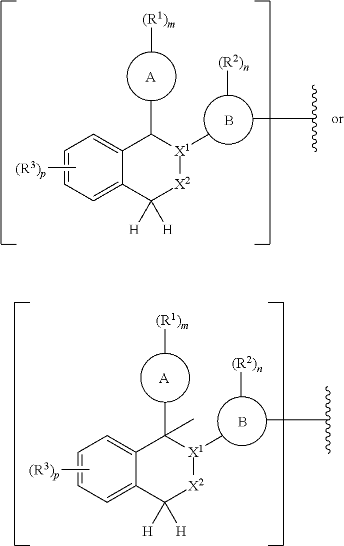

wherein X¹ is CH or N,
   X² is CH₂;
   Ring B is phenyl;
   Ring A is phenyl or a 6 membered monocyclic heteroaryl ring having 1-3 nitrogen heteroatoms;
   at least one R³ is-OH or —OMe;
   p is 1, 2, or 3;
   m is 0, 1 or 2;
   R¹ is selected from —F and —Cl;
   n is 0, 1 or 2; and
   R² is selected from —OH, -Me, —OMe, —F, —Br, —CF₃ and -ⁱPr;
then LBM is not

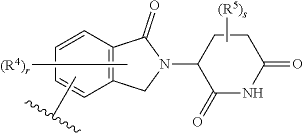

or a stereoisomer thereof.

3. A compound of formula I-3′:

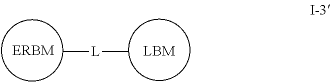

or a pharmaceutically acceptable salt thereof, wherein;
ERBM is selected from

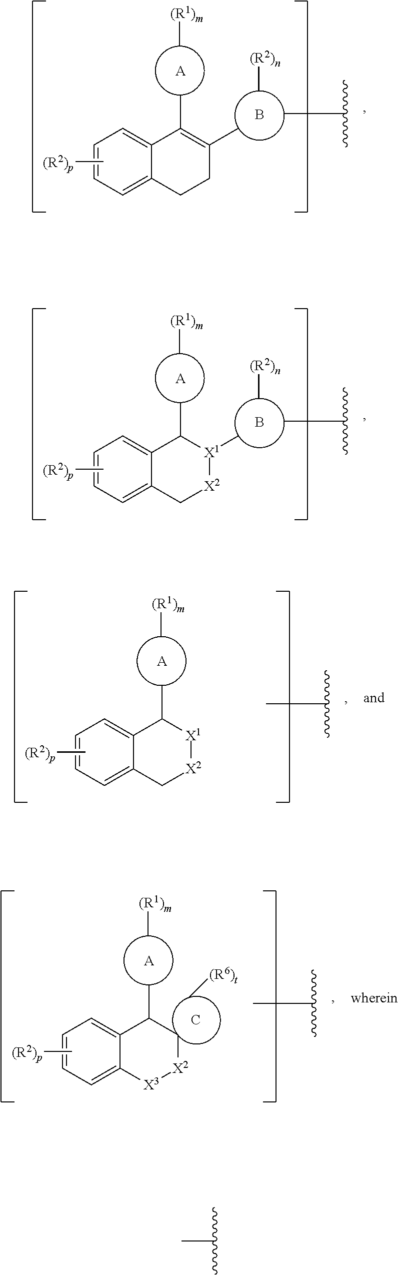

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A5} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

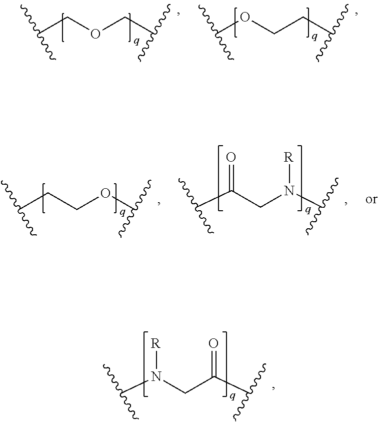

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

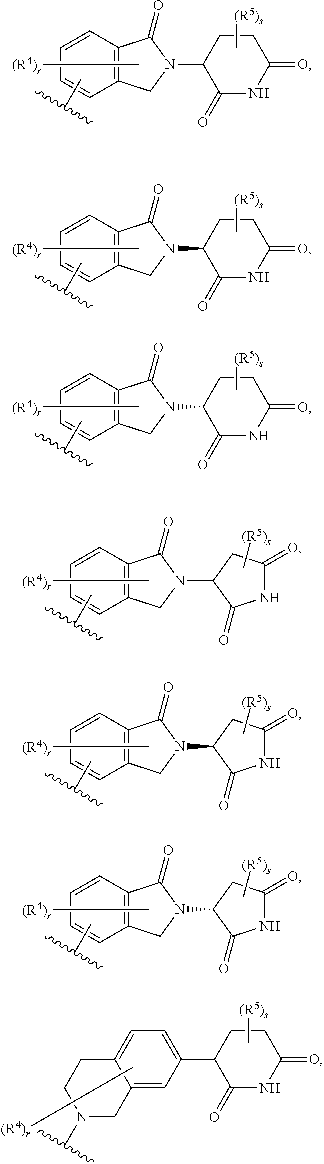

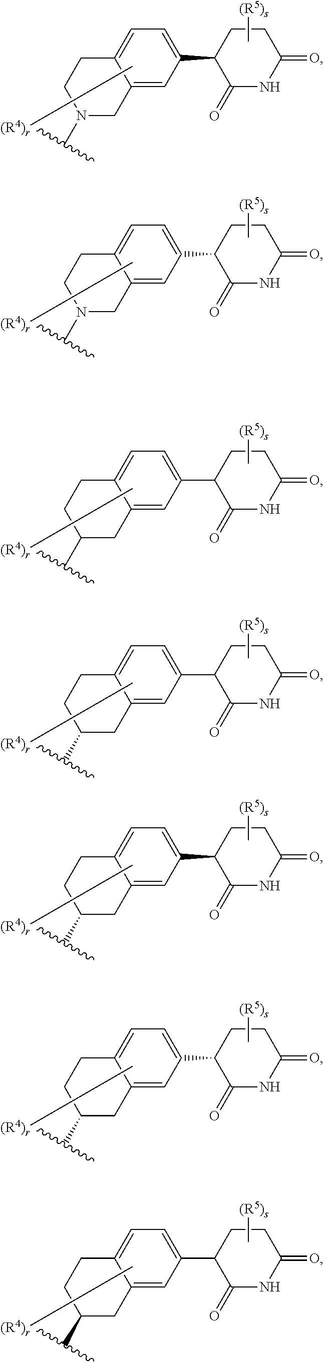

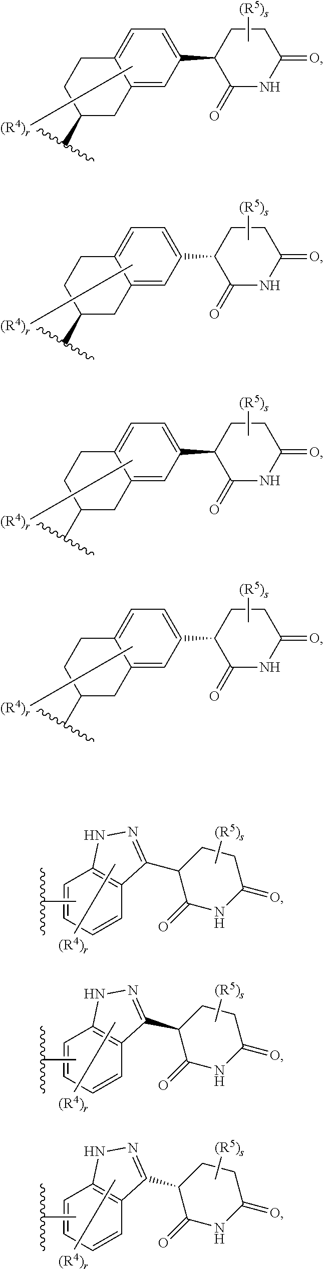

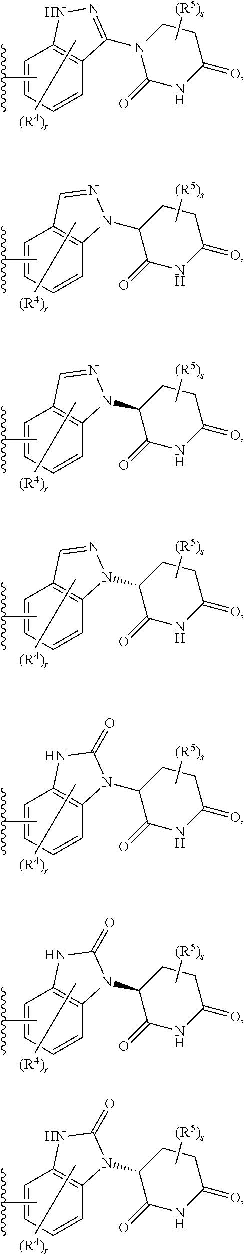

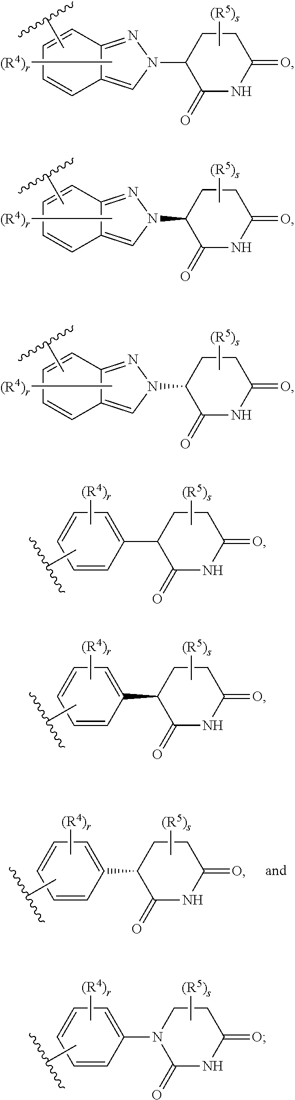

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4,
provided that when ERBM is

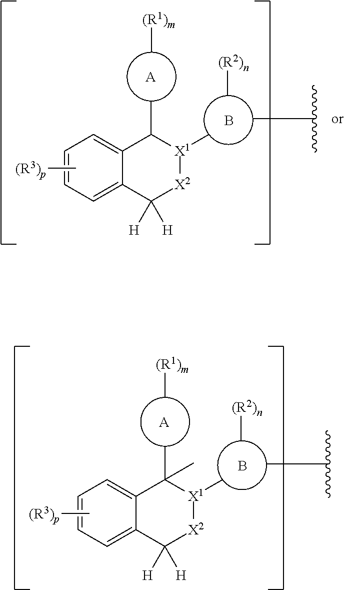

wherein X¹ is CH or N,
   X² is CH₂;
   Ring B is phenyl;
   Ring A is phenyl or a 6 membered monocyclic heteroaryl ring having 1-3 nitrogen heteroatoms;
then R³ is not —OH or —OMe.

4. A compound of formula I-3′:

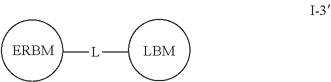

or a pharmaceutically acceptable salt thereof, wherein:
ERBM is selected from

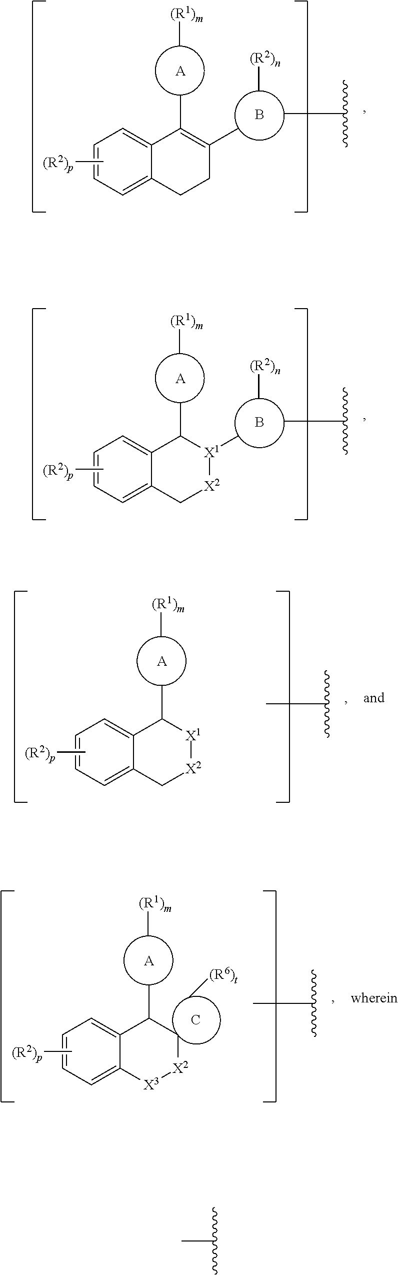

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A5} and R^{A6} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

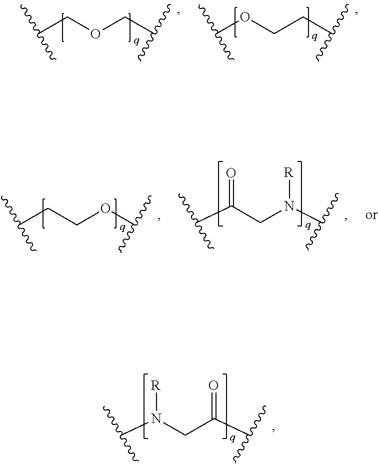

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

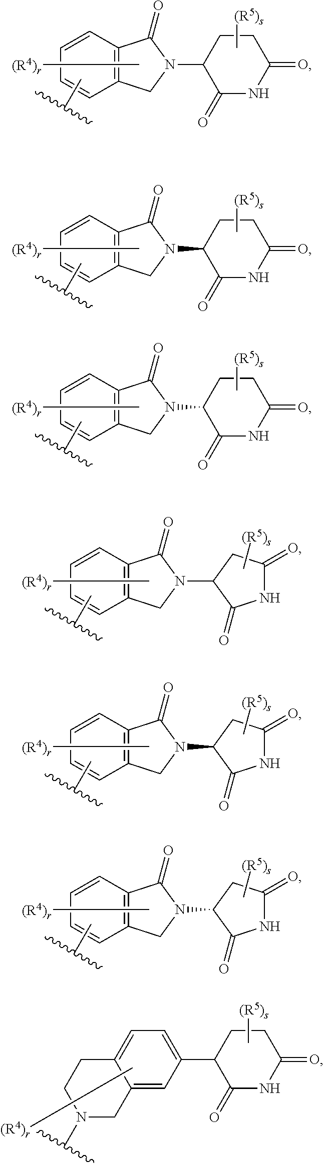

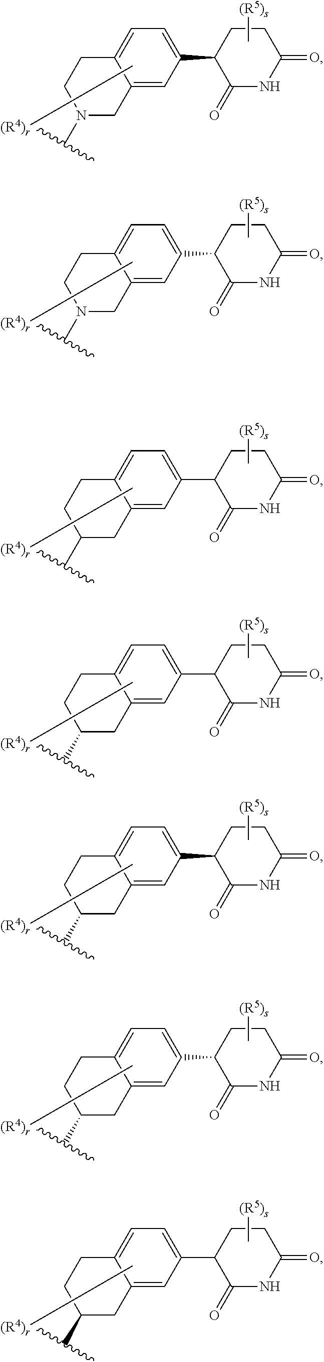

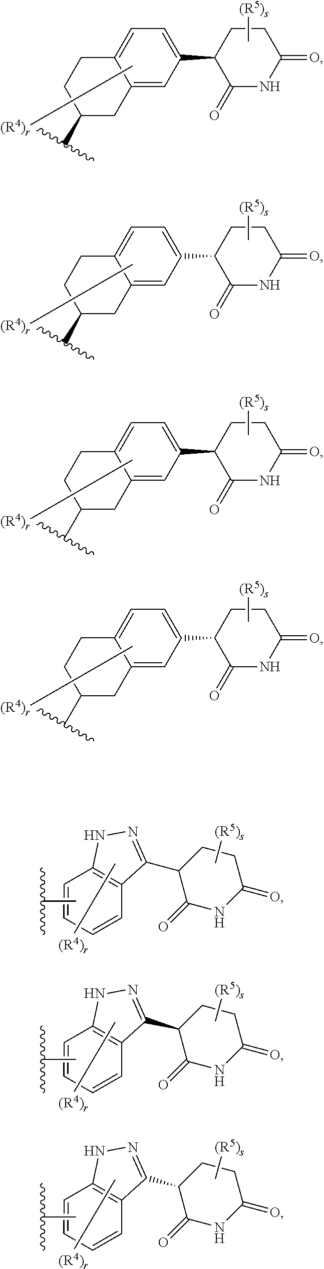

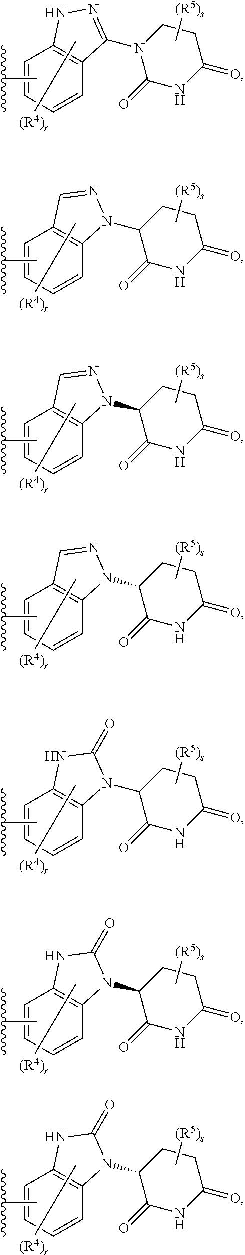

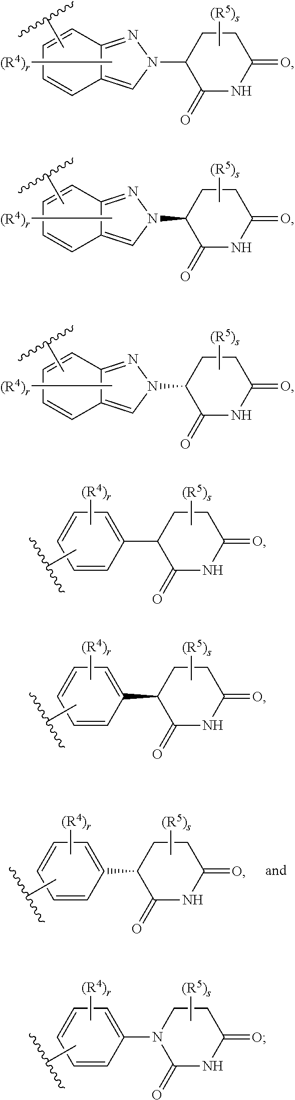

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4,
provided that when ERBM is

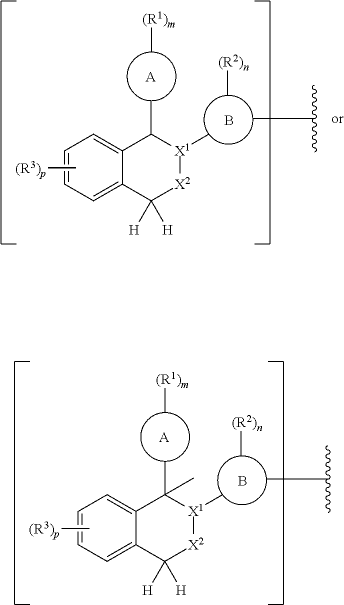

wherein X¹ is CH or N;
   X² is CH₂;
   Ring B is phenyl;
then Ring A is not phenyl or a 6 membered monocyclic heteroaryl ring having 1-3 nitrogen heteroatoms.

5. A compound of formula I-3′:

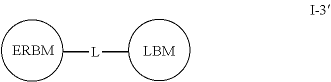

or a pharmaceutically acceptable salt thereof, wherein:
ERBM is selected from

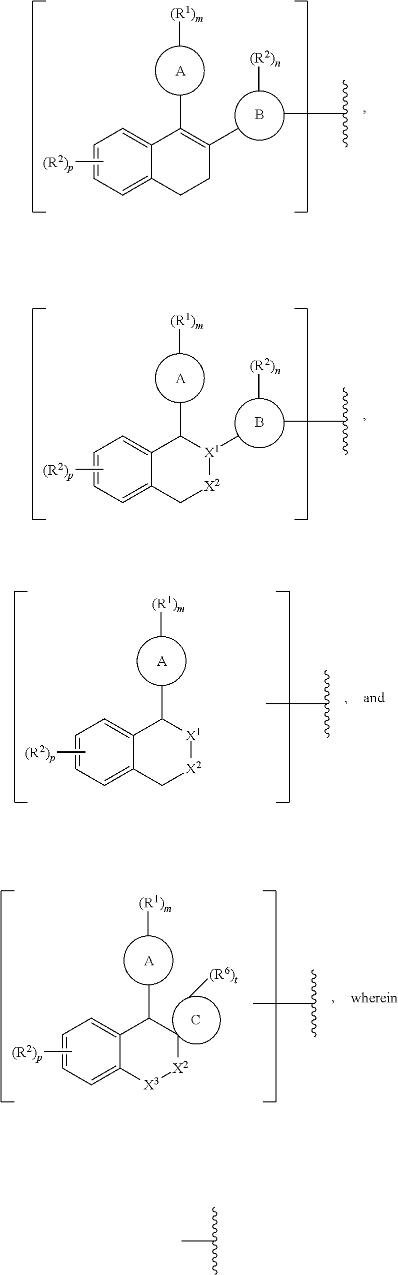

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

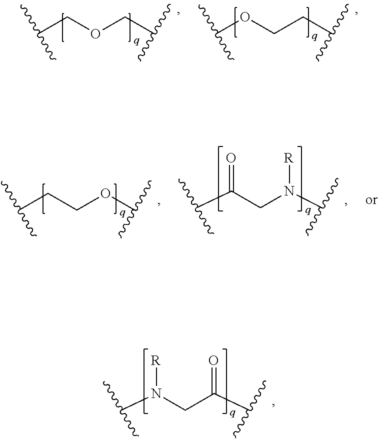

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

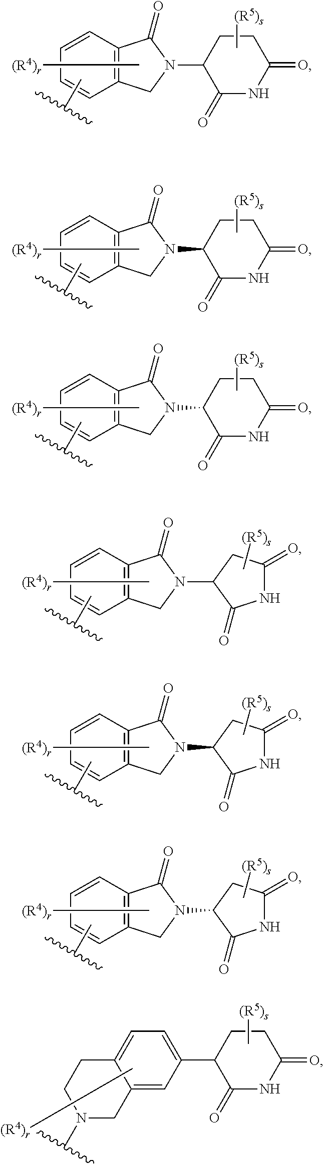

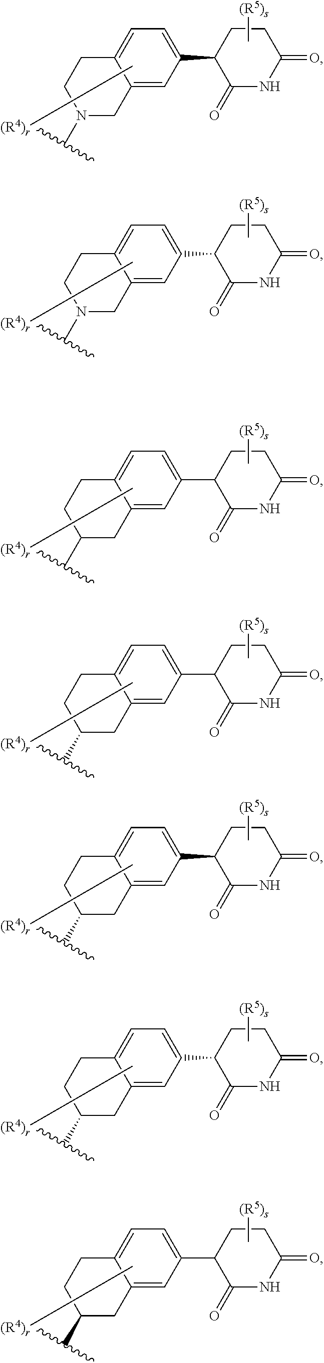

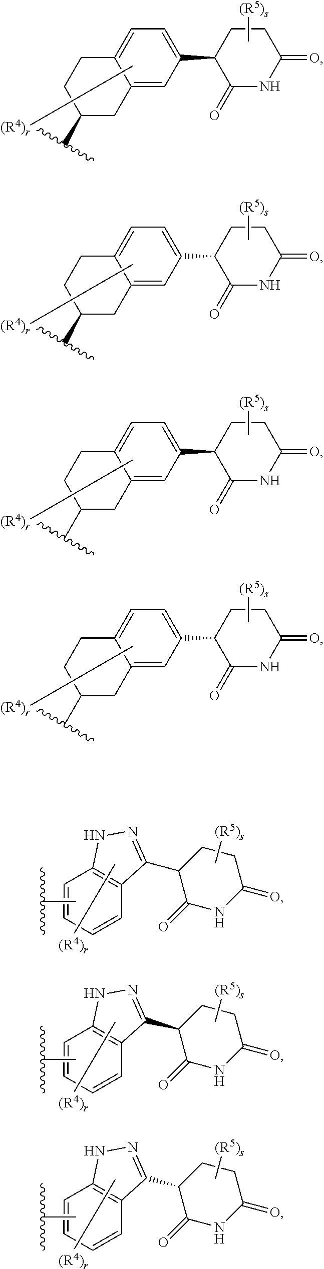

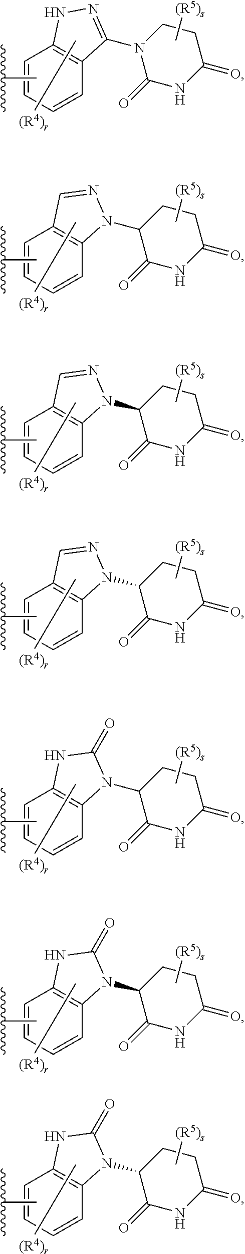

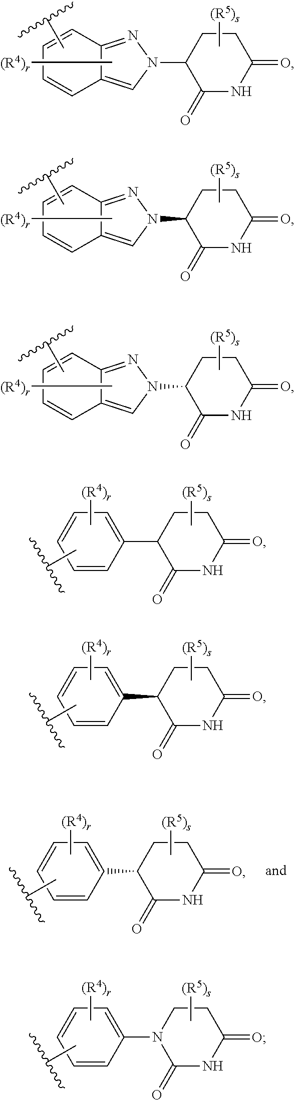

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4,
provided that when ERBM is

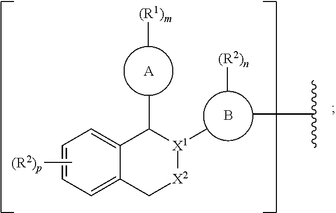

and Ring B is phenyl, X¹ is CH or N and X² is CH₂; then
Ring A is other than phenyl or a 6 membered monocyclic heteroaryl ring having 1-3 nitrogen heteroatoms.

6. A compound of formula I-3:

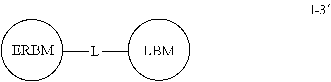

or a pharmaceutically acceptable salt thereof, wherein:
ERBM 18 selected from

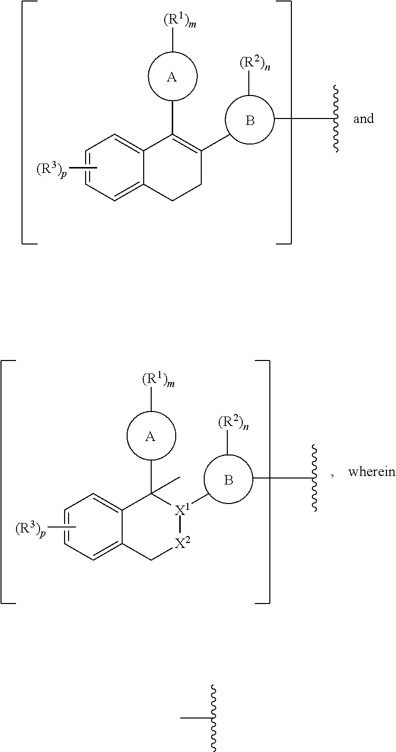

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring 00 are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from independently selected from nitrogen, oxygen, and sulfur; a 3-7 membered saturated or partially bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

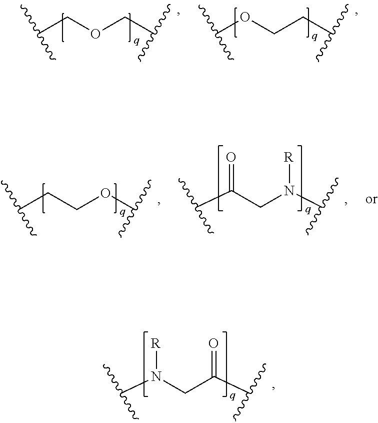

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

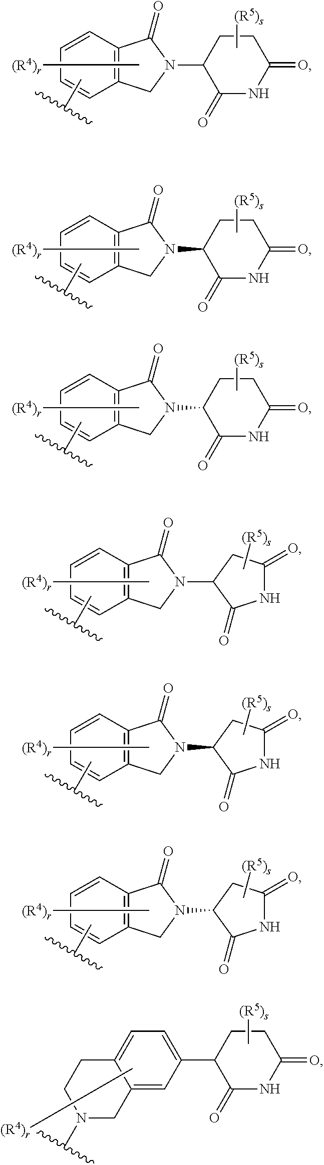

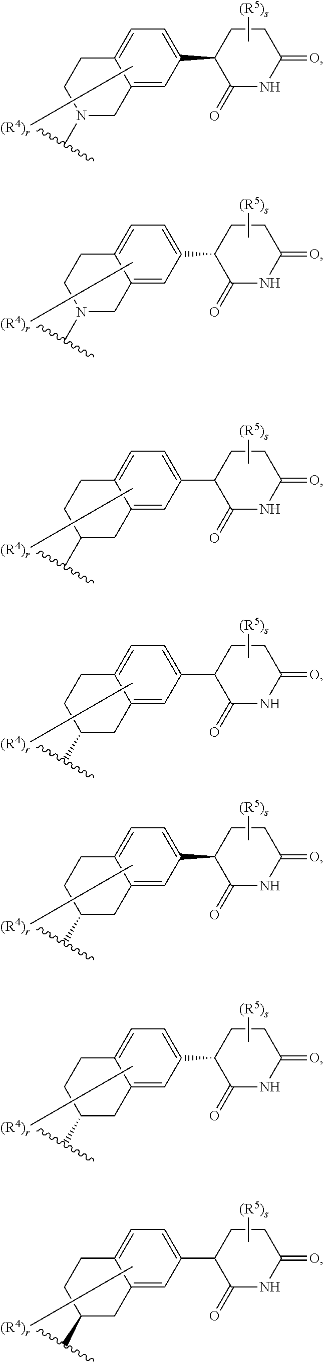

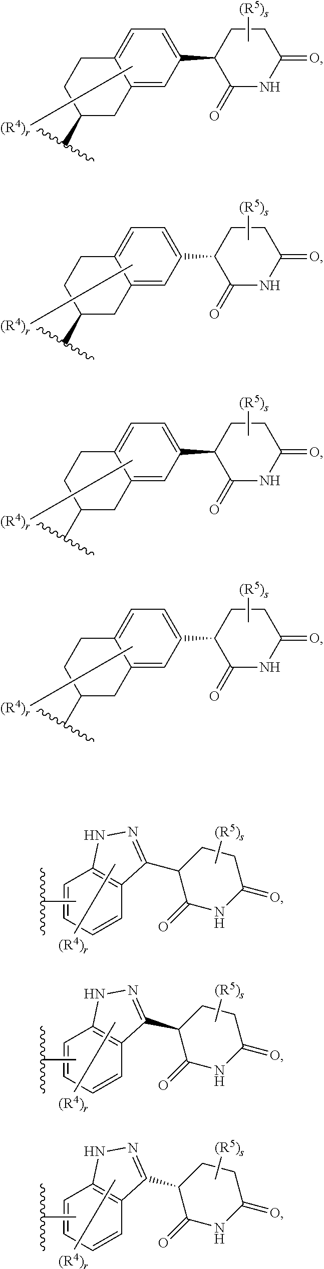

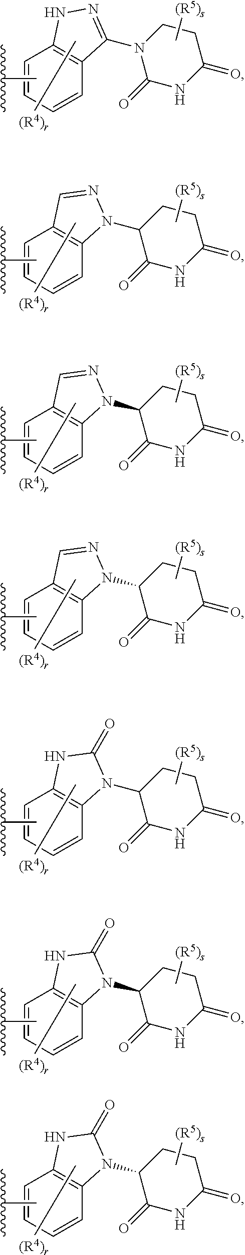

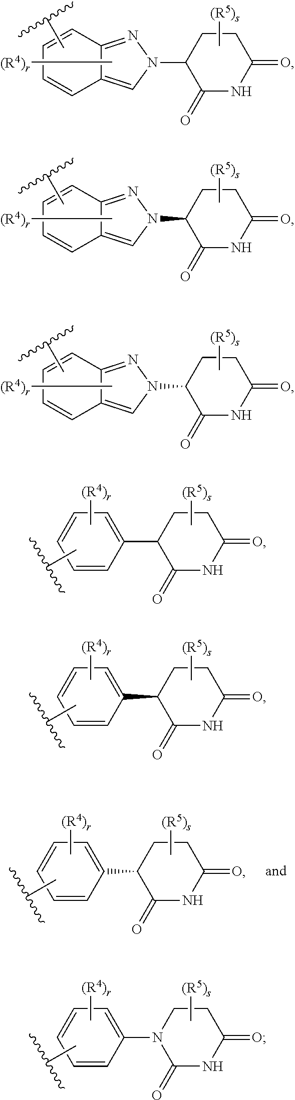

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4.

7. A compound of formula I-3:

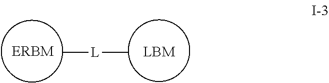

or a pharmaceutically acceptable salt thereof, wherein;
ERBM is selected from

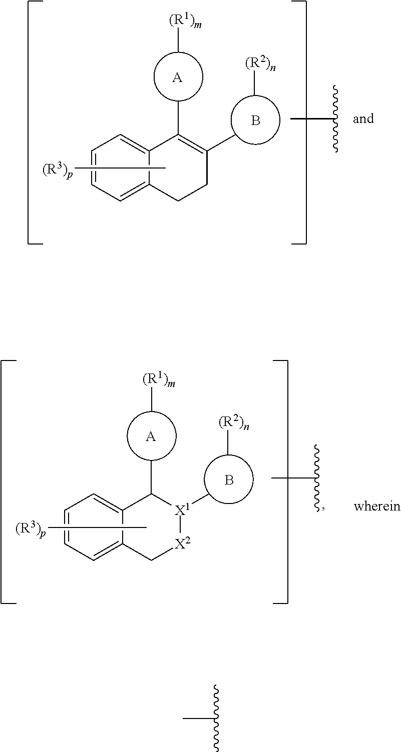

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

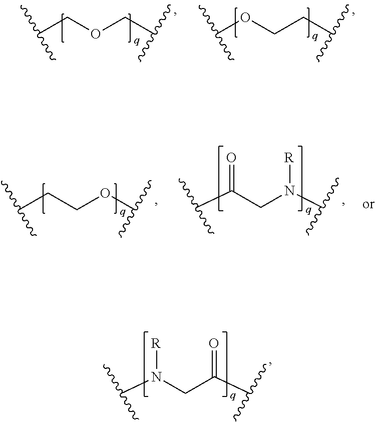

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

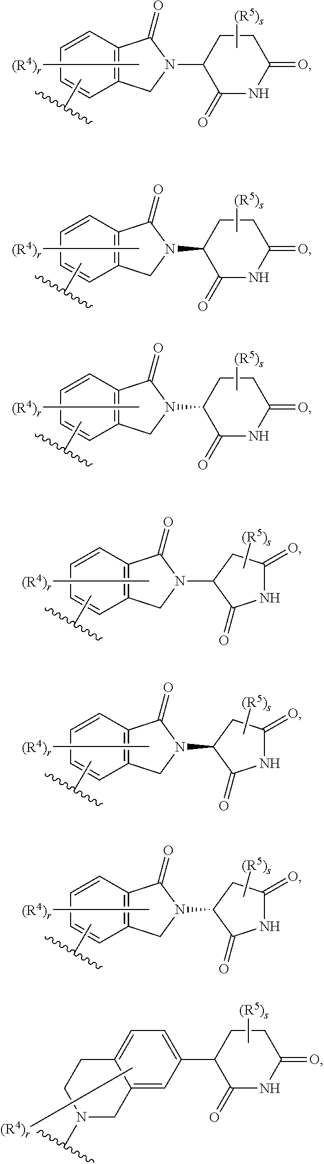

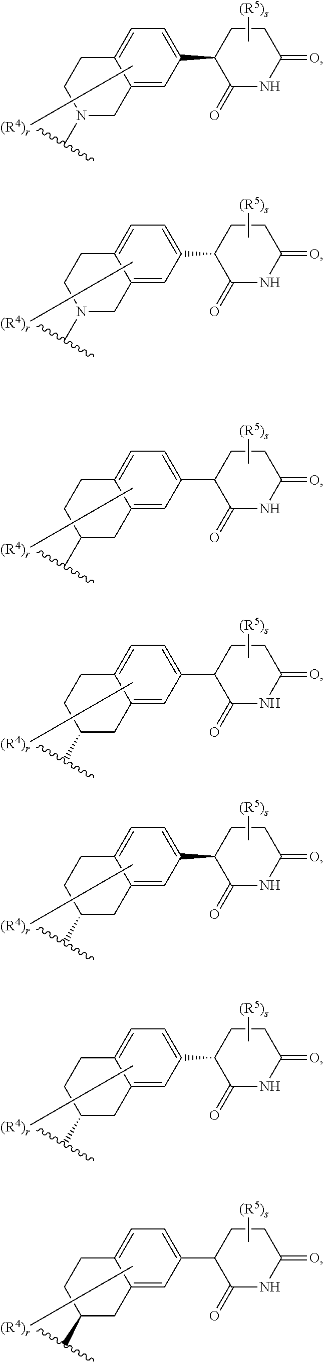

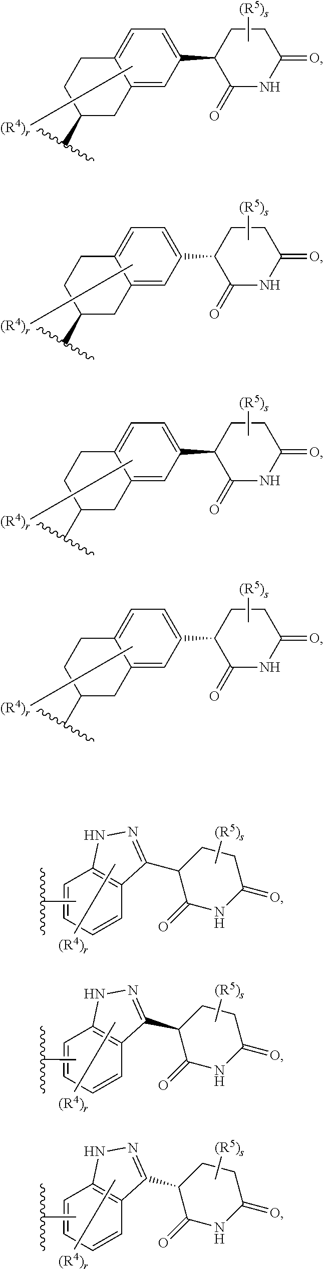

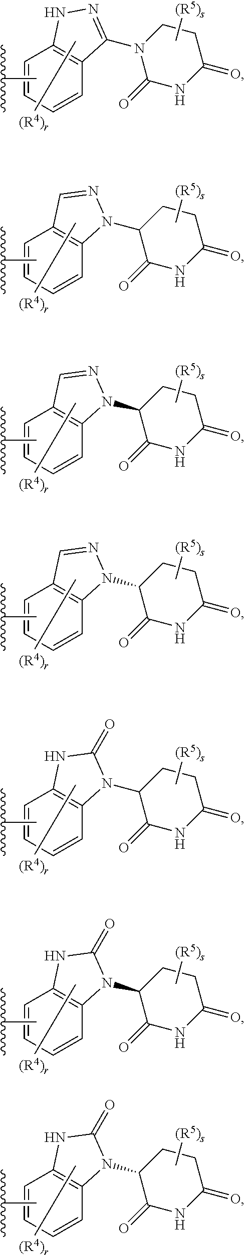

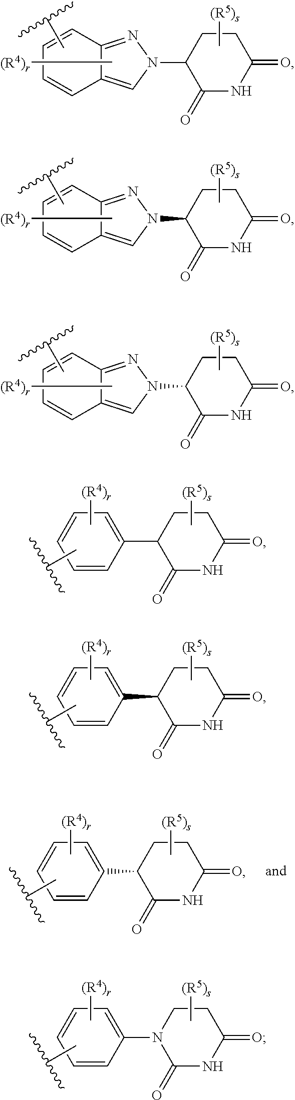

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4,
provided that when ERBM is

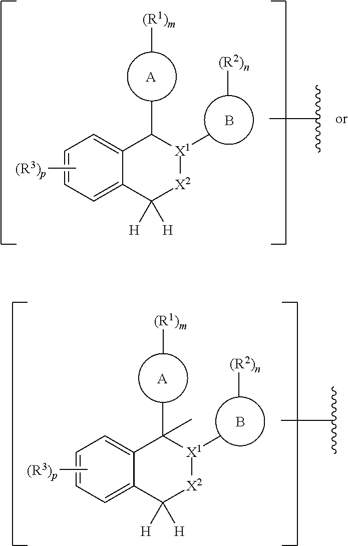

wherein X¹ is CH or N,
   X² is CH₂;
   Ring B is phenyl;
   Ring A is phenyl or a 6 membered monocyclic heteroaryl ring having 1-3 nitrogen heteroatoms;
   at least one R³ is —OH or —OMe;
   p is 1, 2, or 3;
   m is 0, 1 or 2;
   R¹ is selected from —F and —Cl;
   n is 0, 1 or 2; and
   R² is selected from —OH, -Me, —OMe, —F, —Br, —CF; and -ⁱPr;
then LBM is not

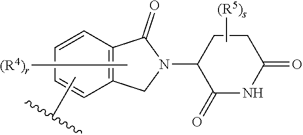

or a stereoisomer thereof.

8. A compound of formula I-3:

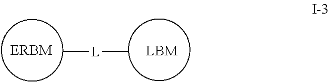

or a pharmaceutically acceptable salt thereof, wherein:
ERBM is selected from

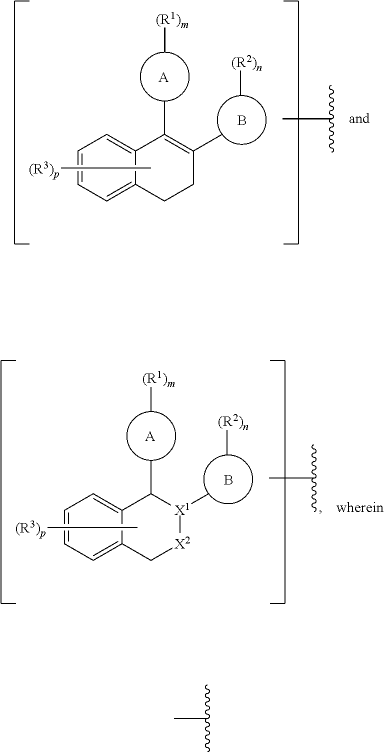

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

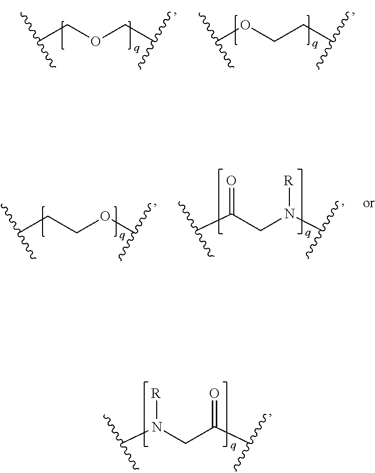

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

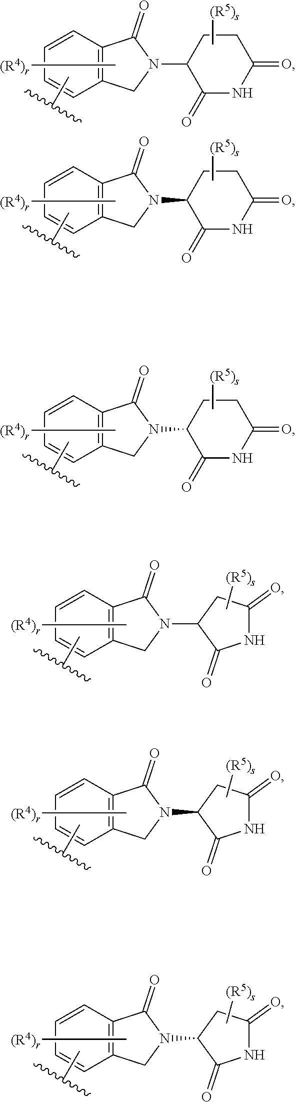

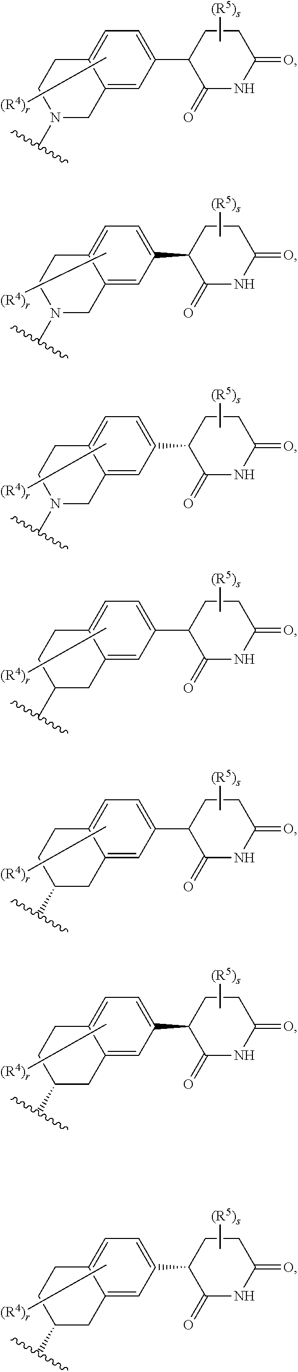

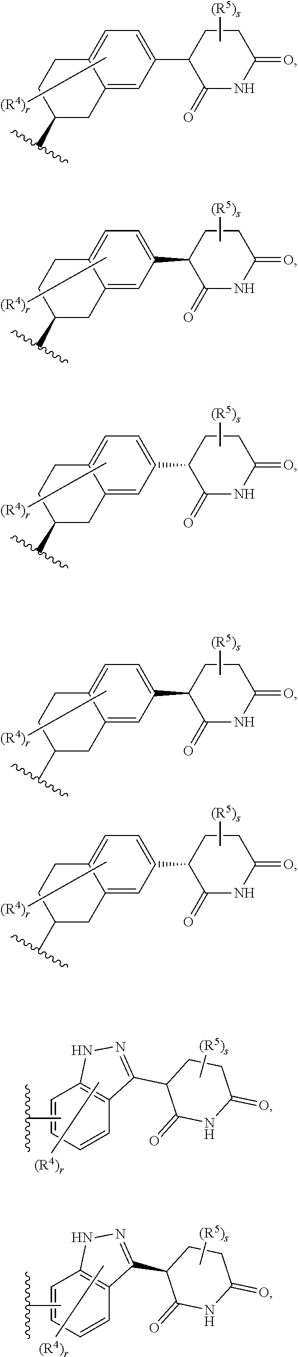

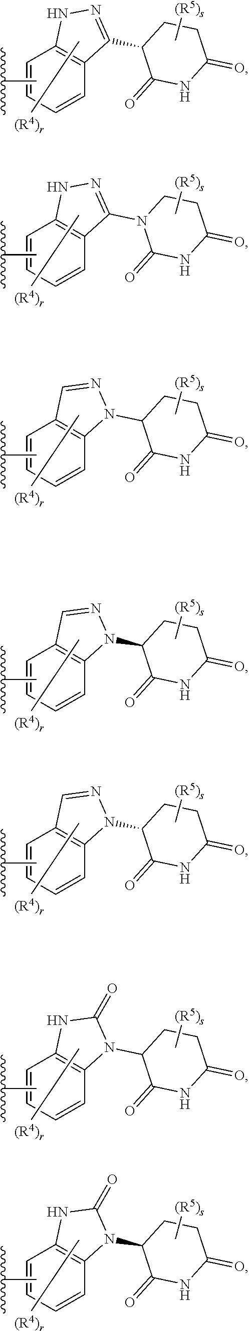

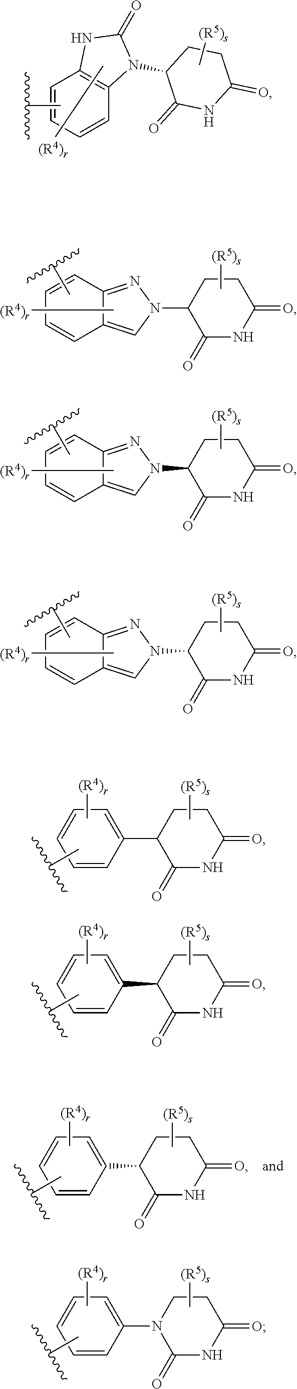

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4,
provided that when ERBM is

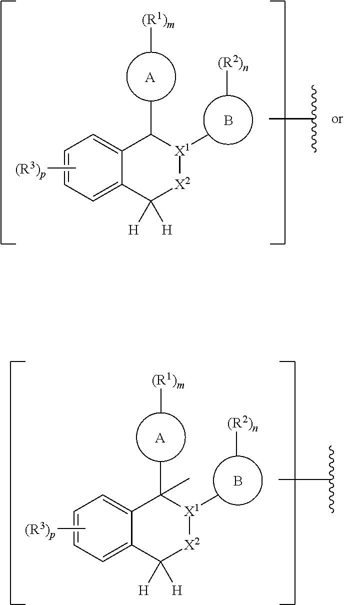

wherein X¹ is CH or N,
   X² is CH₂;
   Ring B is phenyl;
   Ring A is phenyl or a 6 membered monocyclic heteroaryl ring having 1-3 nitrogen heteroatoms;
then R³ is not —OH or —OMe.

9. A compound of formula I-3:

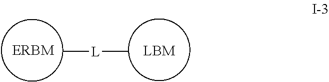

or a pharmaceutically acceptable salt thereof, wherein:
ERBM 18 selected from

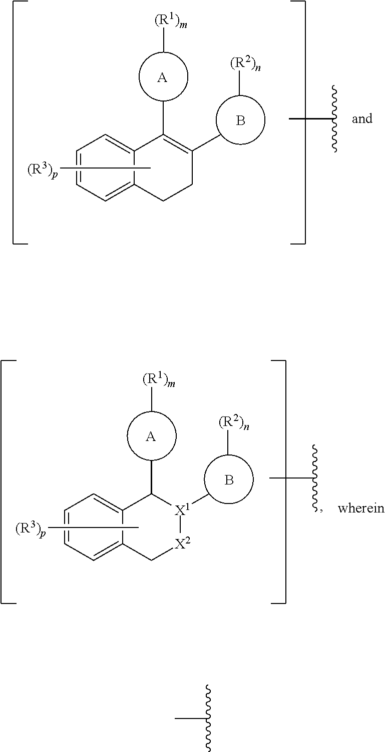

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring 00 are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from independently selected from nitrogen, oxygen, and sulfur; a 3-7 membered saturated or partially bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

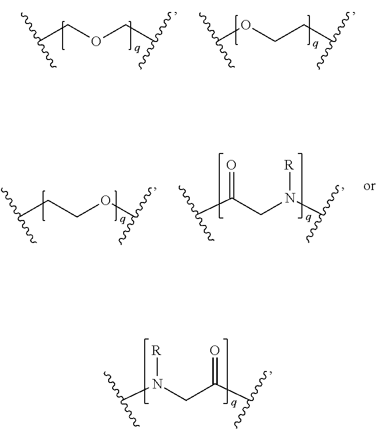

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur; LBM is selected from

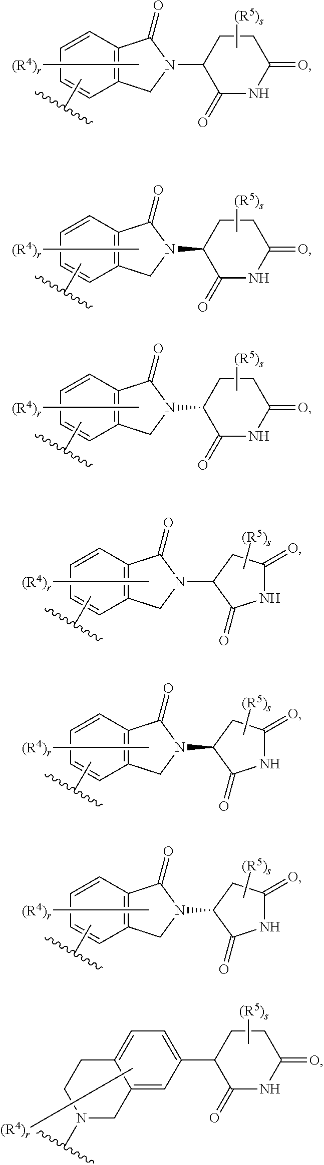

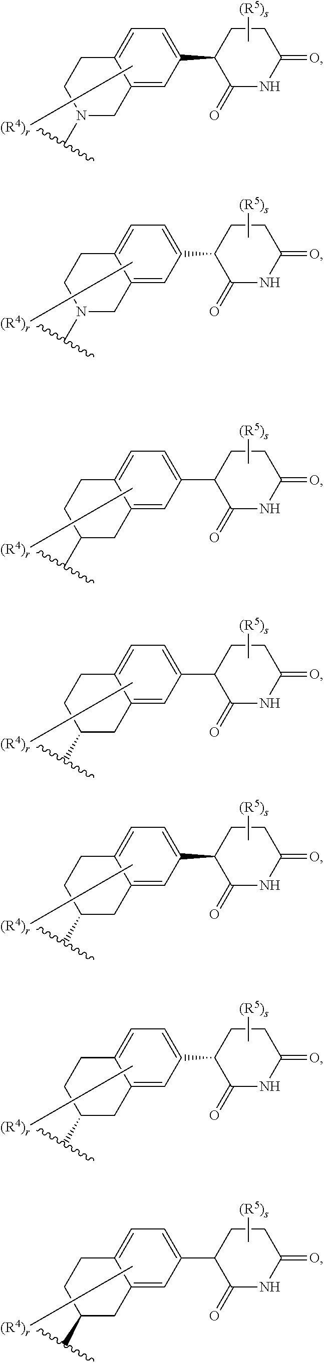

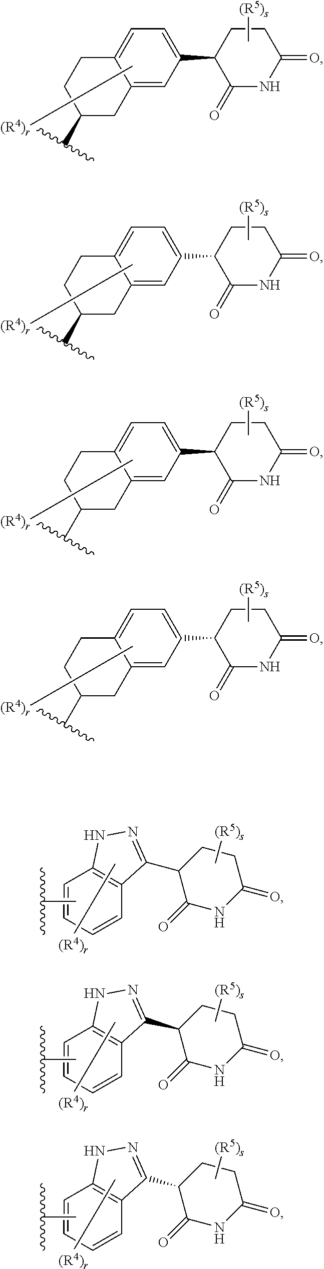

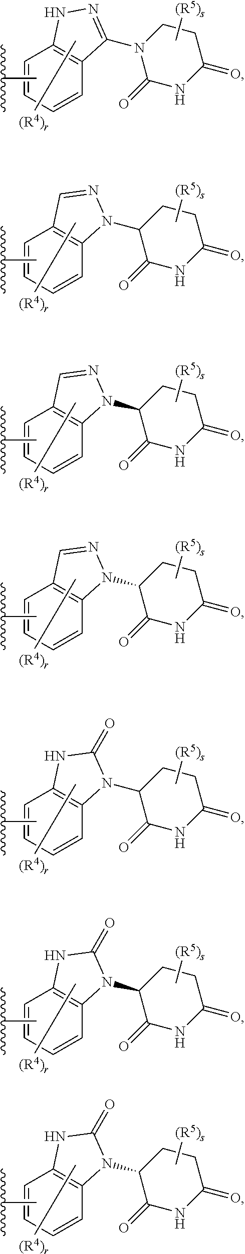

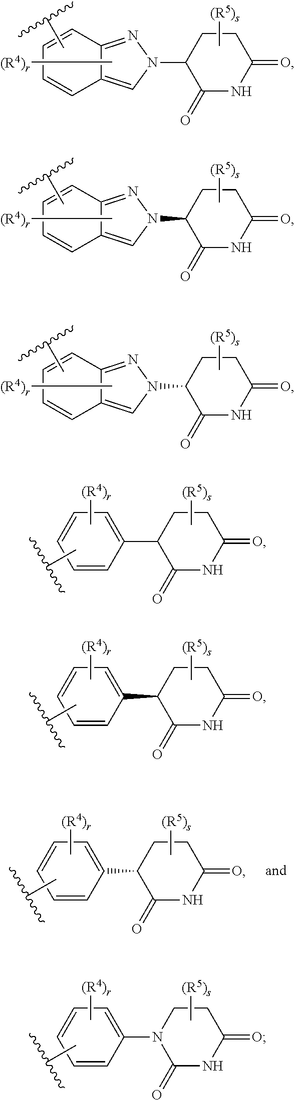

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4,
provided that when ERBM is

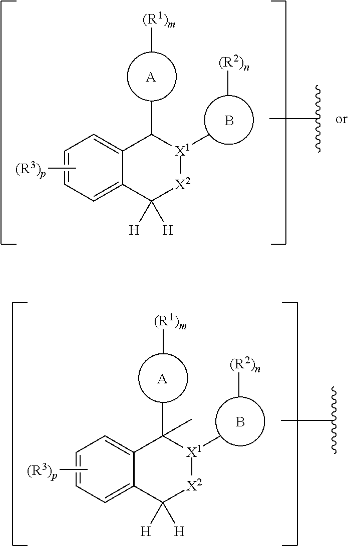

wherein X¹ is CH or N,
   X² is CH₂;
   Ring B is phenyl;
then Ring A is not phenyl or a 6 membered monocyclic heteroaryl ring having 1-3 nitrogen heteroatoms.

10. A compound of formula I-3:

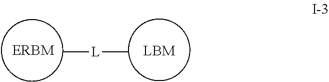

or a pharmaceutically acceptable salt thereof, wherein:
ERBM 18 selected from

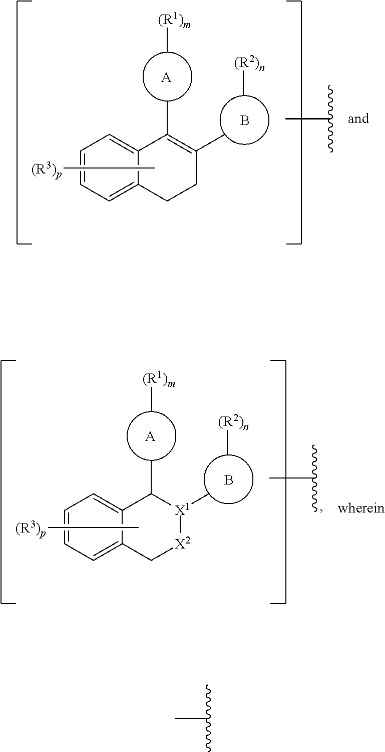

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring 00 are each independently phenyl; 1,1′-biphenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from independently selected from nitrogen, oxygen, and sulfur; a 3-7 membered saturated or partially bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

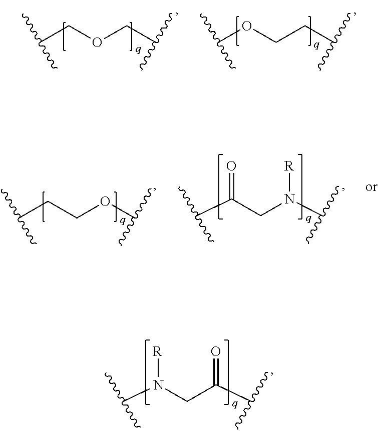

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

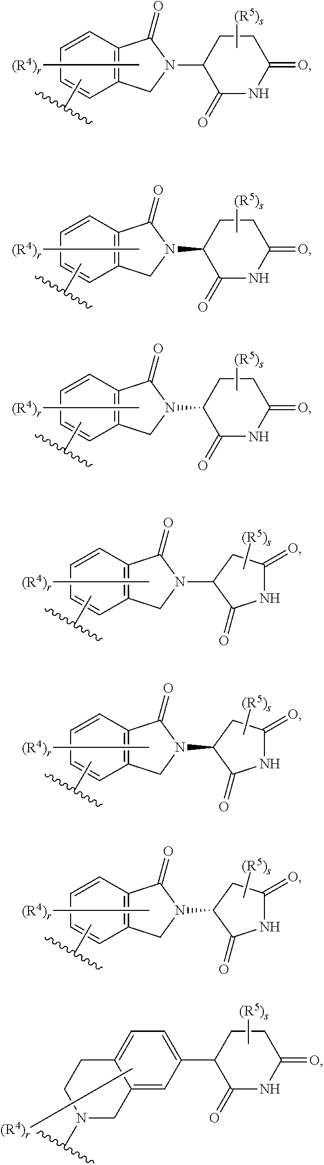

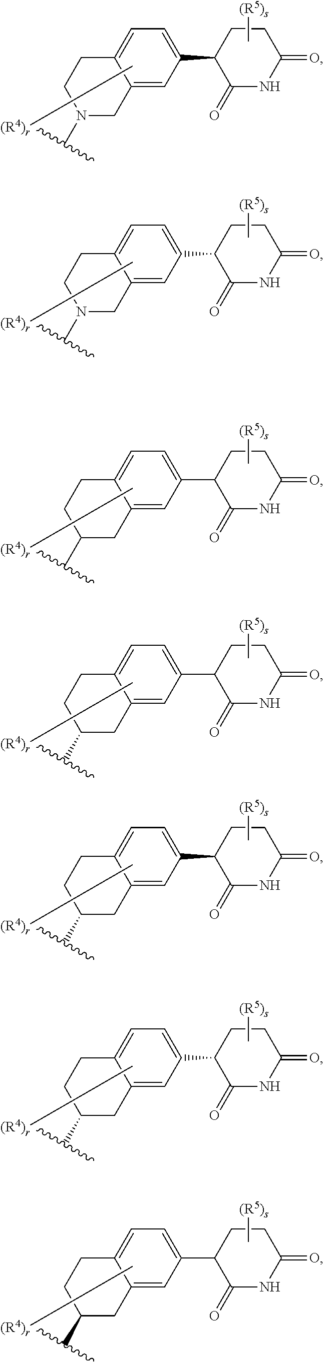

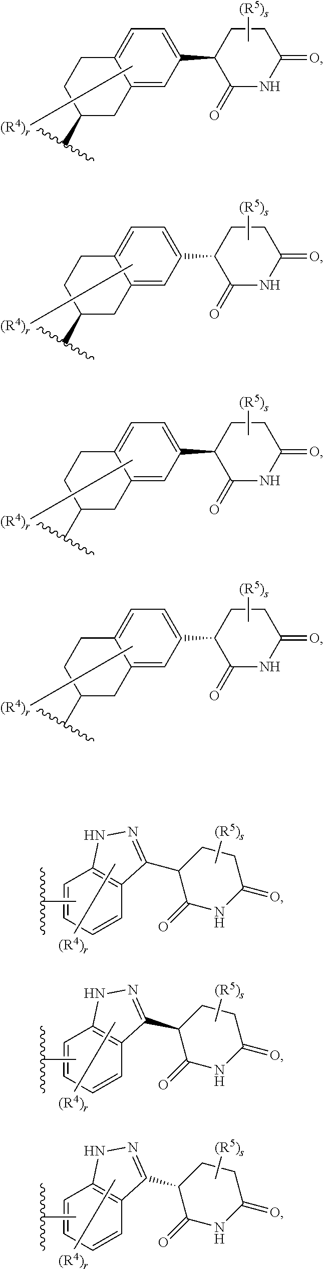

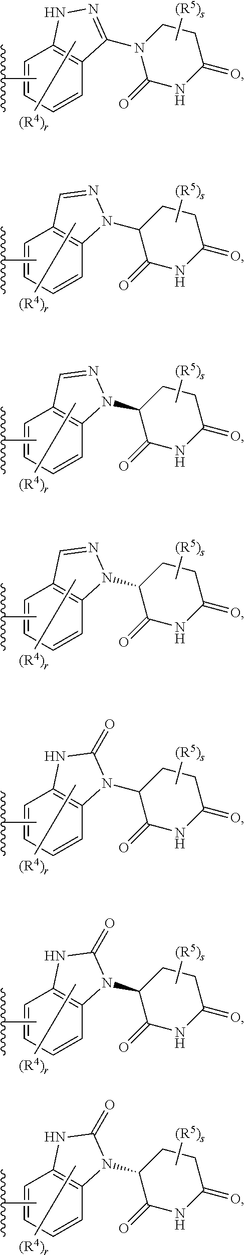

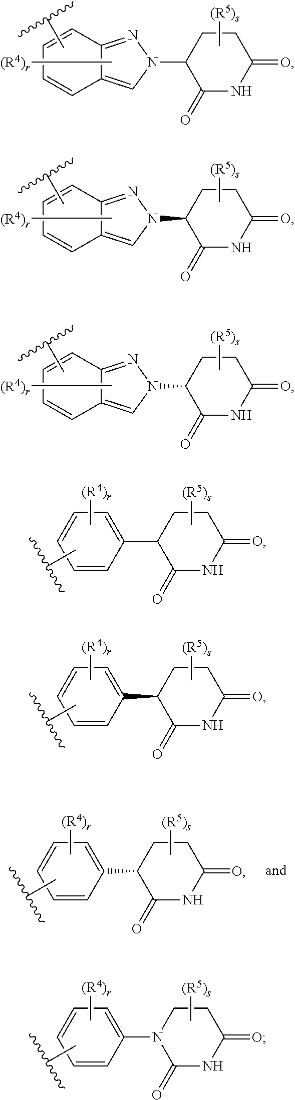

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4,
provided that when ERBM is

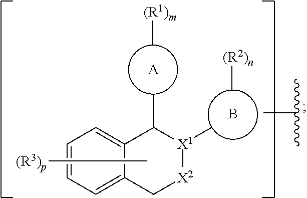

and Ring B is phenyl, X¹ is CH or N and X² is CH₂; then
Ring A is other than phenyl or a 6 membered monocyclic heteroaryl ring having 1-3 nitrogen heteroatoms.

11. A compound of formula I-3:

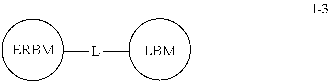

or a pharmaceutically acceptable salt thereof, wherein:
ERBM is selected from

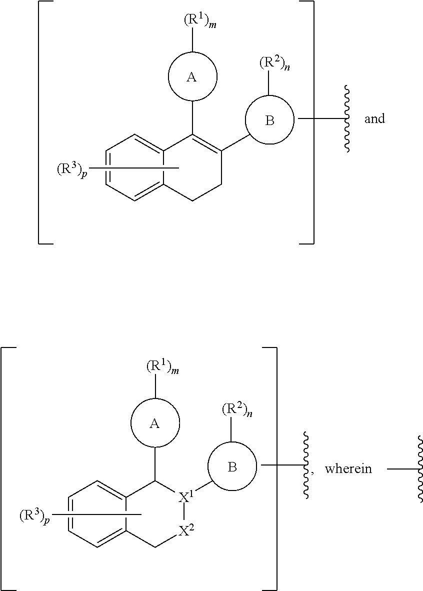

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl;
a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O) ¿R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

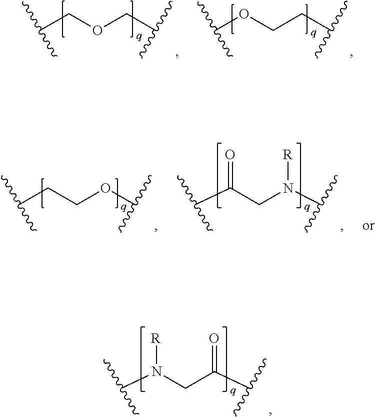

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

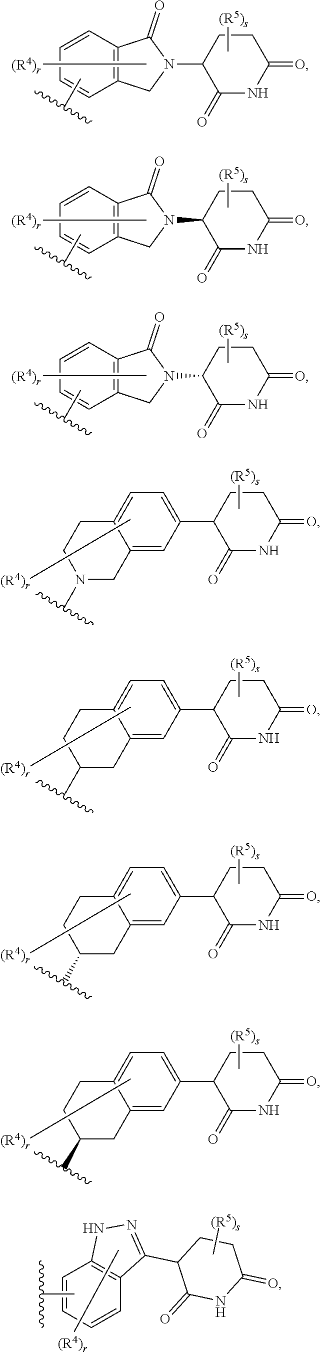

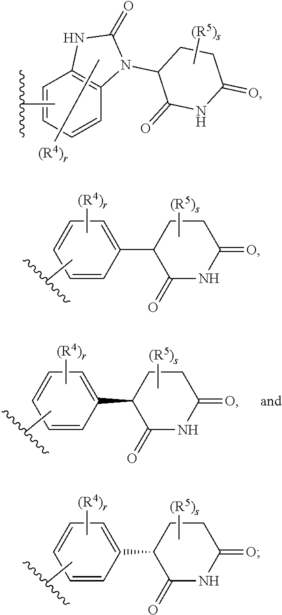

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4.

12. A compound of formula I-3:

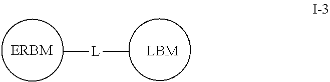

or a pharmaceutically acceptable salt thereof, wherein:
ERBM is selected from

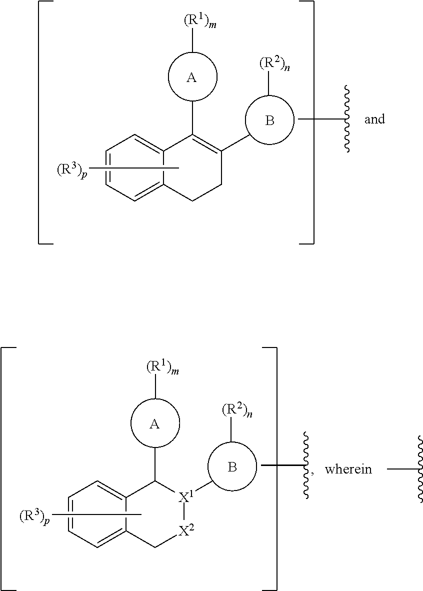

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R⁴³), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

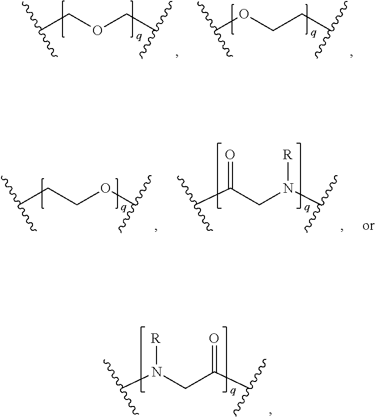

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

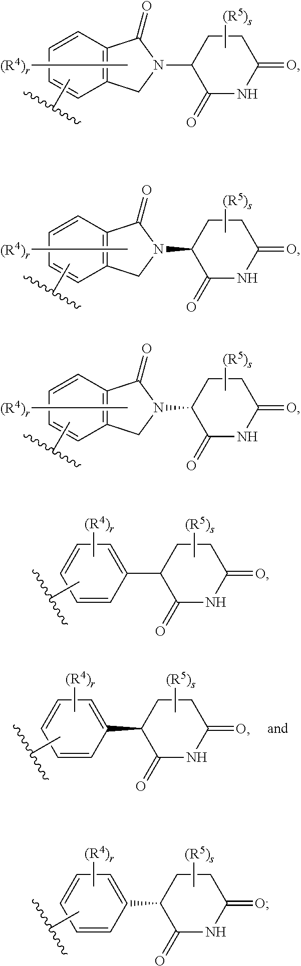

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4.

13. A compound of formula I-3′:

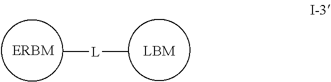

or a pharmaceutically acceptable salt thereof, wherein:
ERBM is selected from

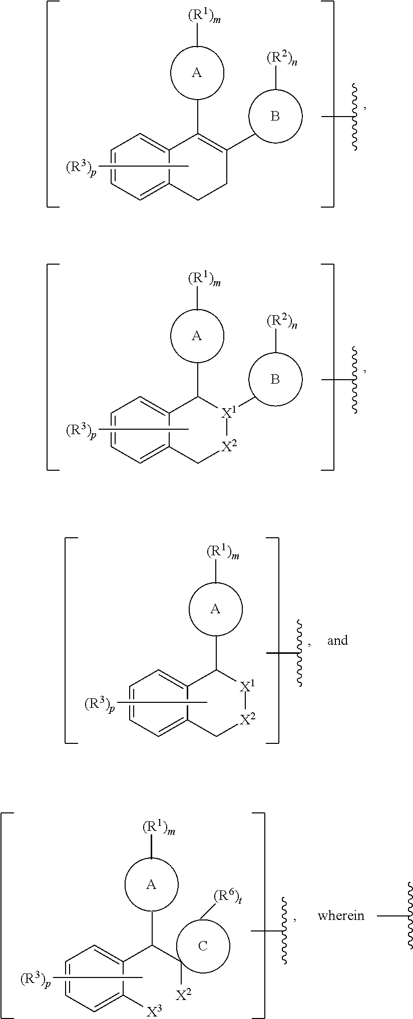

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O) 2R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

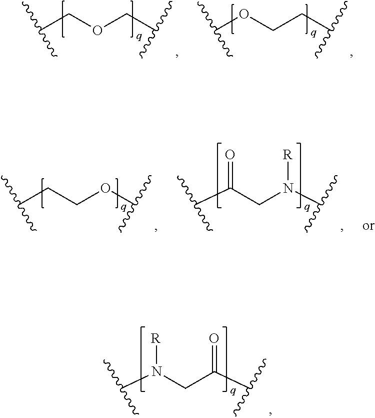

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

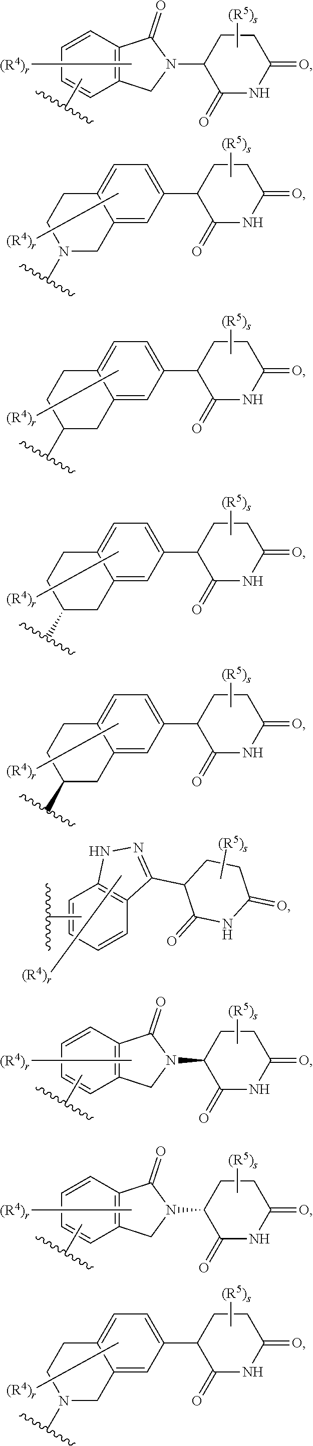

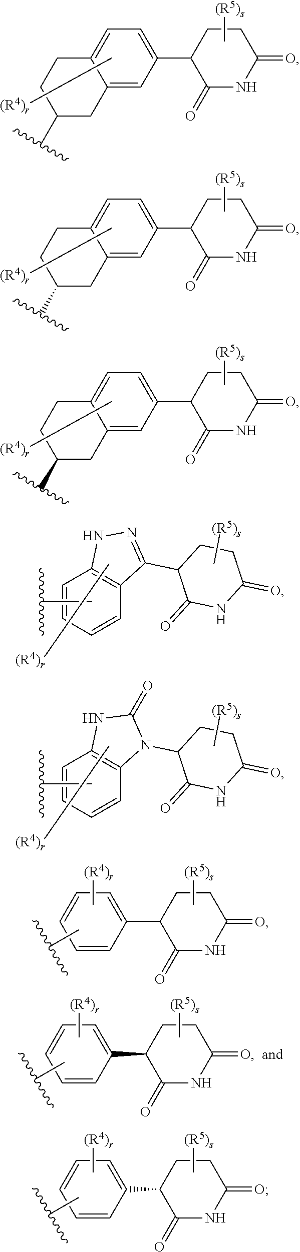

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

14. A compound of formula I-3′:

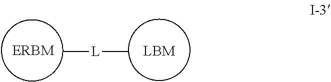

or a pharmaceutically acceptable salt thereof, wherein:
ERBM is selected from

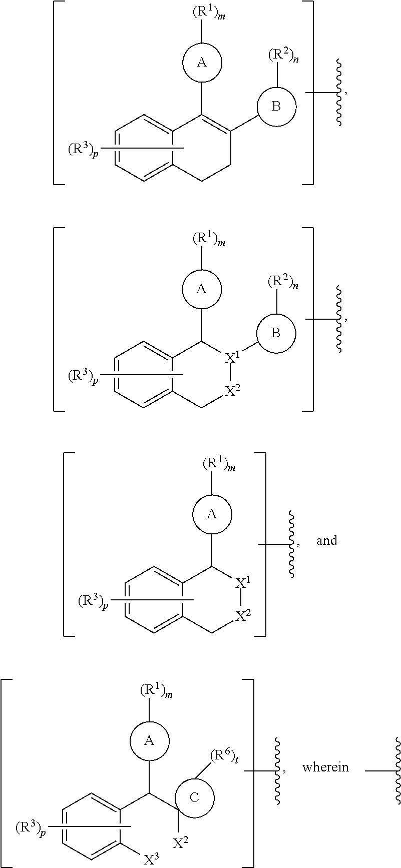

indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

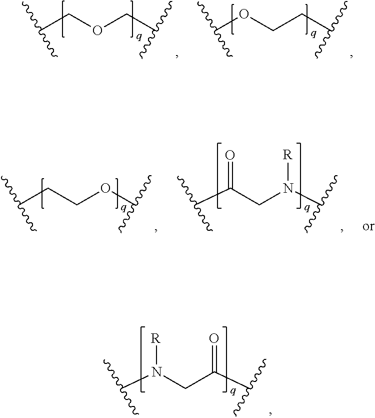

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

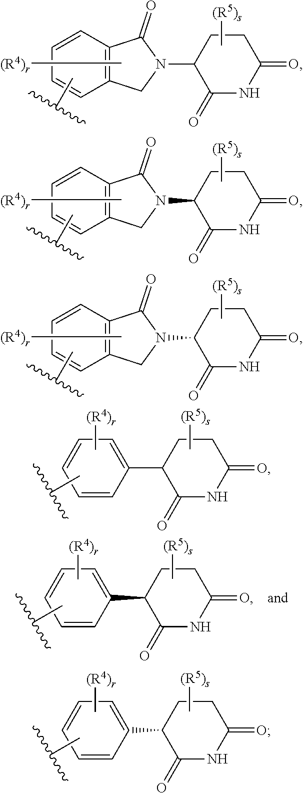

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

15. A compound of formula I-3:

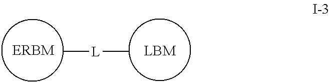

or a pharmaceutically acceptable salt thereof, wherein:
ERBM 18 selected from

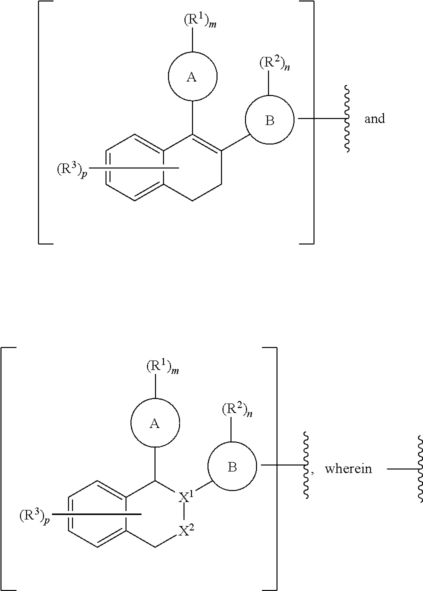

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, R^{A1}, R^{A2}, and R^{A3} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

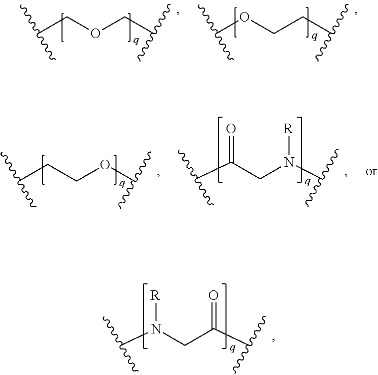

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

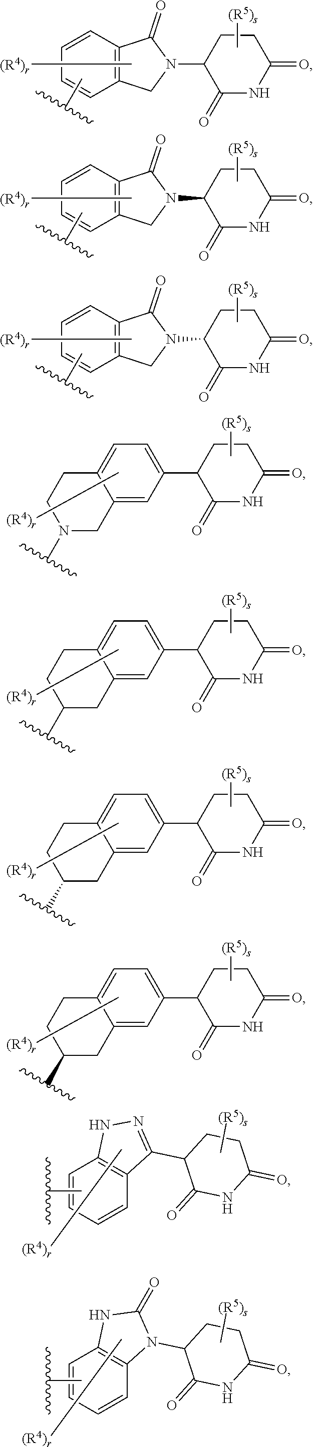

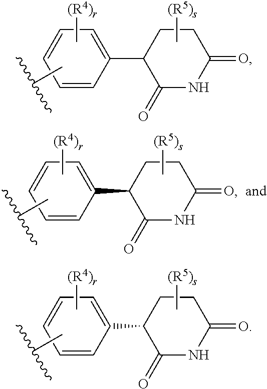

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4;
provided that when ERBM is

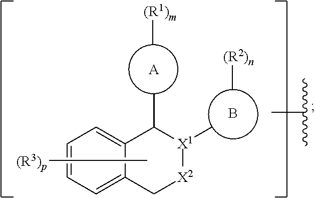

and Ring B is phenyl, X¹ is CH or N and X² is CH₂; then
Ring A is other than phenyl or a 6 membered monocyclic heteroaryl ring having 1-3 nitrogen heteroatoms.

16. A compound of formula I-3:

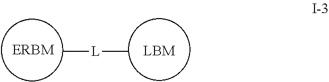

or a pharmaceutically acceptable salt thereof, wherein:
ERBM is selected from

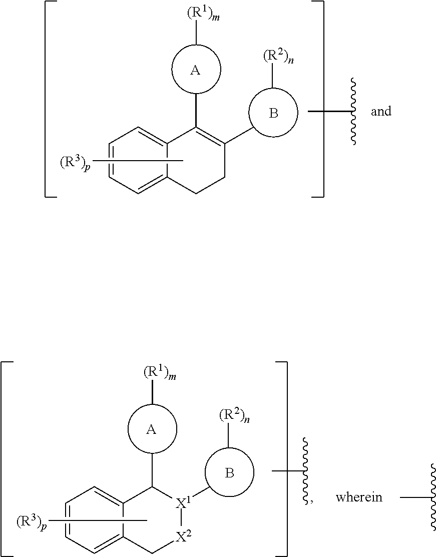

indicates the site of attachment of the -L-LBM moiety to a modifiable carbon, oxygen, nitrogen, or sulfur atom of the ERBM moiety;
X¹ is N, CH, or C(R^{A1});
X² is N(R^{A2}), O, CH₂, CH(R^{A3}), or C(R^{A3})₂;
provided that X¹ and X² are not both heteroatoms;
each instance of R¹, R², R⁴, R⁵, RAL, R^{A2}, and R^{A3} is independently R^{A} or R^{B}, and is substituted by 0-4 instances of R^{C}:
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O) OR, —C(O)NR₂, —C(O)N(R) OR, —OC(O)R, —OC(O)NR₂, —N(R)C(O) OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, or —B(OR)₂;
each instance of R^{B} is independently a C₁₋₆ aliphatic chain; phenyl; naphthyl; cubanyl;
adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O) OR, —C(O)NR₂, —C(O)N(R) OR, —OC(O)R, —OC(O)NR₂, —N(R)C(O) OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

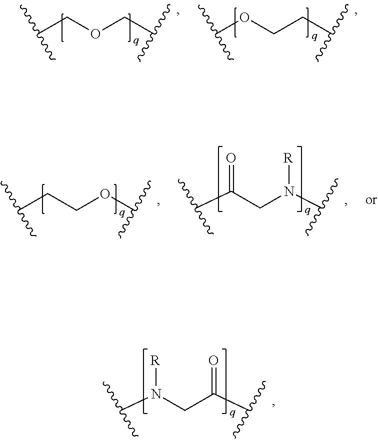

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

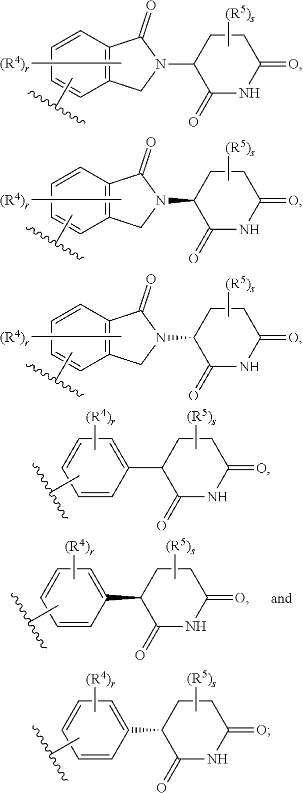

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4; and
s is 0, 1, 2, 3, or 4;
provided that when ERBM is

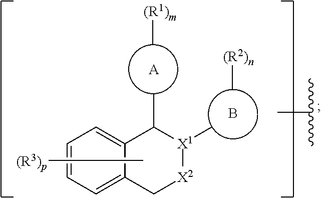

and
Ring B is phenyl, X¹ is CH or N and X² is CH₂; then
Ring A is other than phenyl or a 6 membered monocyclic heteroaryl ring having 1-3 nitrogen heteroatoms.

17. A compound of formula I-3′:

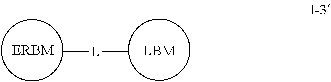

or a pharmaceutically acceptable salt thereof, wherein:
ERBM is selected from

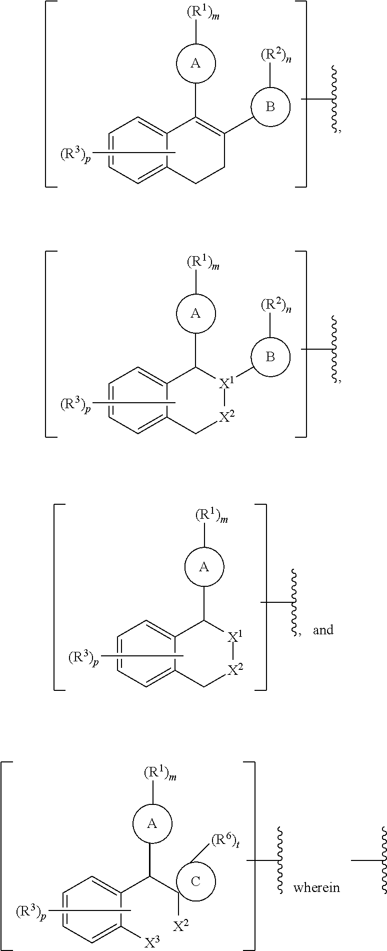

indicates the site of attachment of the -L-LBM moiety to a modifiable carbon, oxygen, nitrogen, or sulfur atom of the ERBM moiety;
X¹ is N, NH, CH, CH₂, CH(R^{A1}), C(R^{A1}), or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O) OR, —C(O)NR₂, —C(O)N(R) OR, —OC(O)R, —OC(O)NR₂, —N(R)C(O) OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O) OR, —C(O)NR₂, —C(O)N(R) OR, —OC(O)R, —OC(O)NR₂, —N(R)C(O) OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

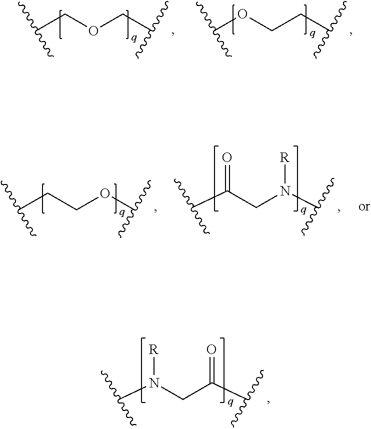

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

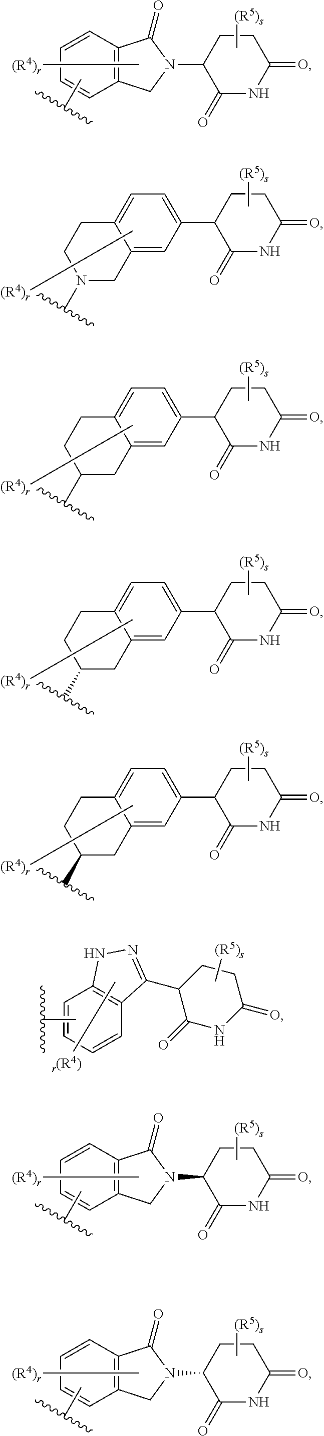

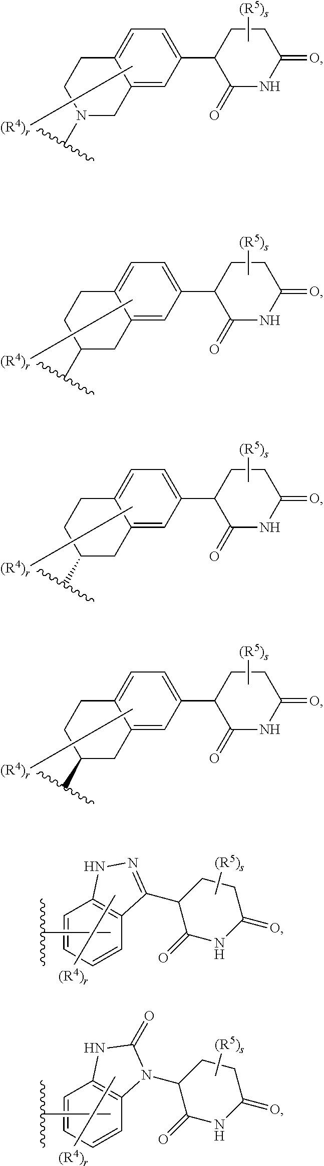

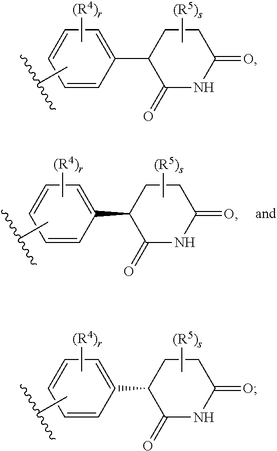

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4;
provided that when ERBM is

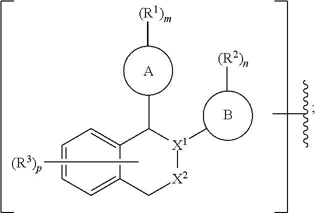

and Ring B is phenyl, X¹ is CH or N and X² is CH₂; then
   Ring A is other than phenyl or a 6 membered monocyclic heteroaryl ring having 1-3 nitrogen heteroatoms.

18. A compound of formula I-3′:

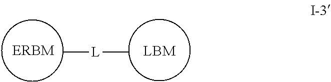

or a pharmaceutically acceptable salt thereof, wherein:
ERBM is selected from

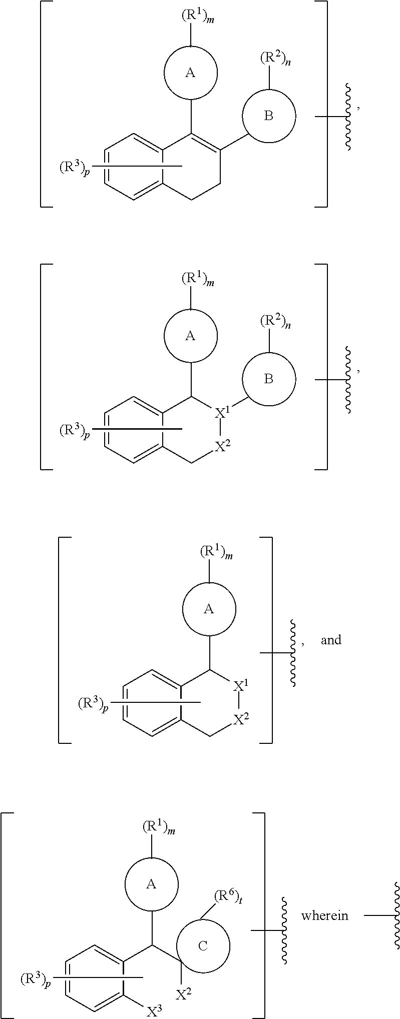

indicates the site of attachment of the -L-LBM moiety to a modifiable carbon, oxygen, nitrogen, or sulfur atom of the ERBM moiety;
X¹ is N, NH, CH, CH₂, CH(R^{A1}), C(R^{A1}), or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O) OR, —C(O)NR₂, —C(O)N(R) OR, —OC(O)R, —OC(O)NR₂, —N(R)C(O) OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O) OR, —C(O)NR₂, —C(O)N(R) OR, —OC(O)R, —OC(O)NR₂, —N(R)C(O) OR, —N(R)C(O)R, —N(R)C(O)NR₂, —N(R)C(NR)NR₂, —N(R)S(O)₂NR₂, —N(R)S(O)₂R, —P(O)R₂, —P(O)(R) OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)—, —NRS(O)₂—, —S(O)NR—, —NRC(O)—, —C(O)NR—, —OC(O)NR—, —NRC(O)O—,

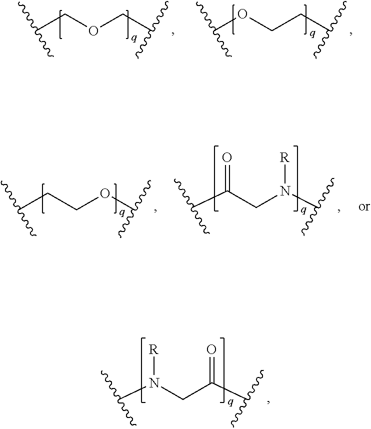

wherein:
   each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from

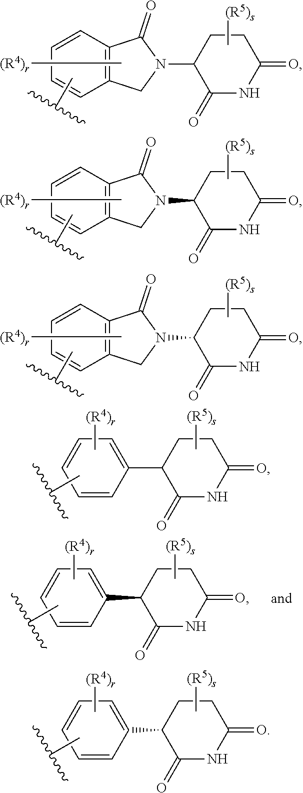

m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4;
provided that when ERBM is

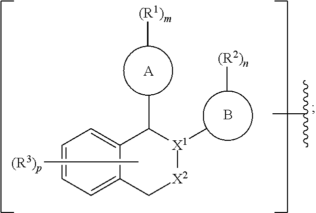

and Ring B is phenyl, X¹ is CH or N and X² is CH₂; then
Ring A is other than phenyl or a 6 membered monocyclic heteroaryl ring having 1-3 nitrogen heteroatoms.

19. The compound of any one of embodiments 1-18, wherein ERBM is

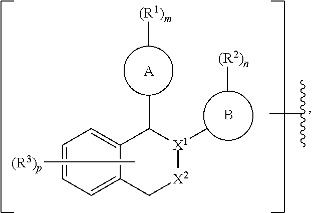

or a pharmaceutically acceptable salt thereof.

20. The compound of any one of embodiments 1-18, wherein ERBM is

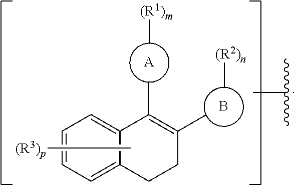

or a pharmaceutically acceptable salt thereof.

21. The compound of any one of embodiments 1-18, wherein ERBM is

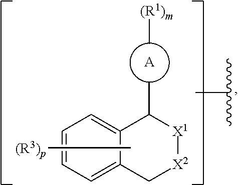

or a pharmaceutically acceptable salt thereof.

22. The compound of any one of embodiments 1-18, wherein ERBM is

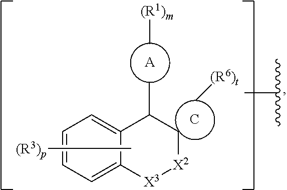

or a pharmaceutically acceptable salt thereof.

23. The compound of any one of embodiments 1-18, wherein the compound is a compound of formula II-a, II-b, or II-c:

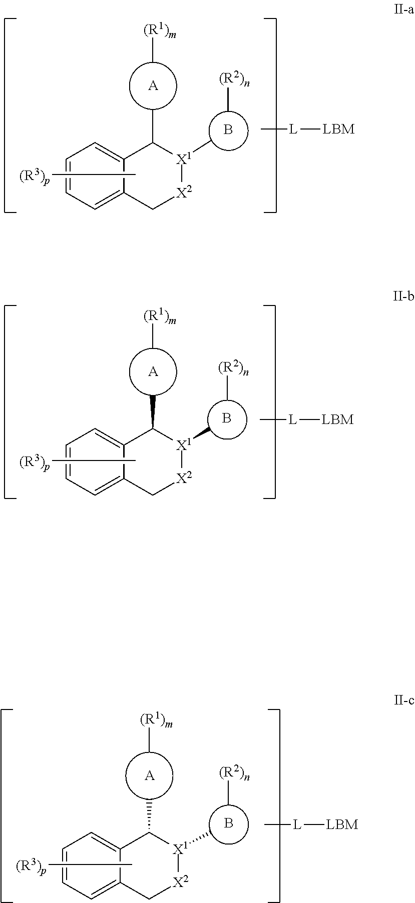

or a pharmaceutically acceptable salt thereof.

24. The compound of any one of embodiments 1-18, wherein the compound is a compound of formula II-a-1, II-b-1, or II-c-1:

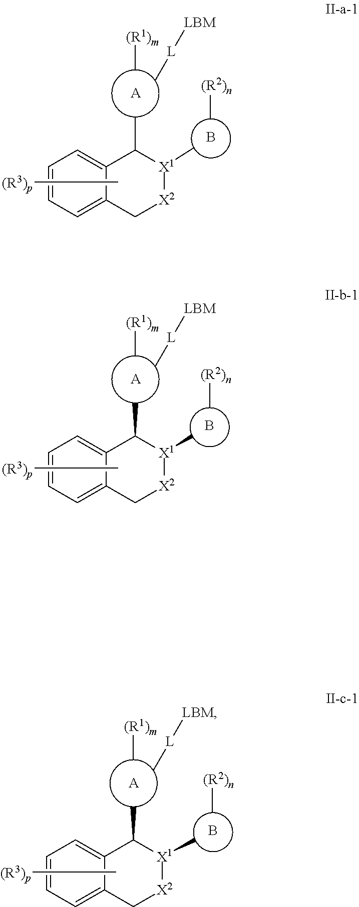

or a pharmaceutically acceptable salt thereof.

25. The compound of embodiment 24, wherein the compound is a compound of formula II-a-1, or a pharmaceutically acceptable salt thereof.

26. The compound of embodiment 24, wherein the compound is a compound of formula II-b-1, or a pharmaceutically acceptable salt thereof.

27. The compound of embodiment 24, wherein the compound is a compound of formula II-c-1, or a pharmaceutically acceptable salt thereof.

28. The compound of any one of embodiments 1-18, wherein the compound is a compound of formula VIII-a, VIII-b, VIII-c, IX-a, IX-b or IX-c:

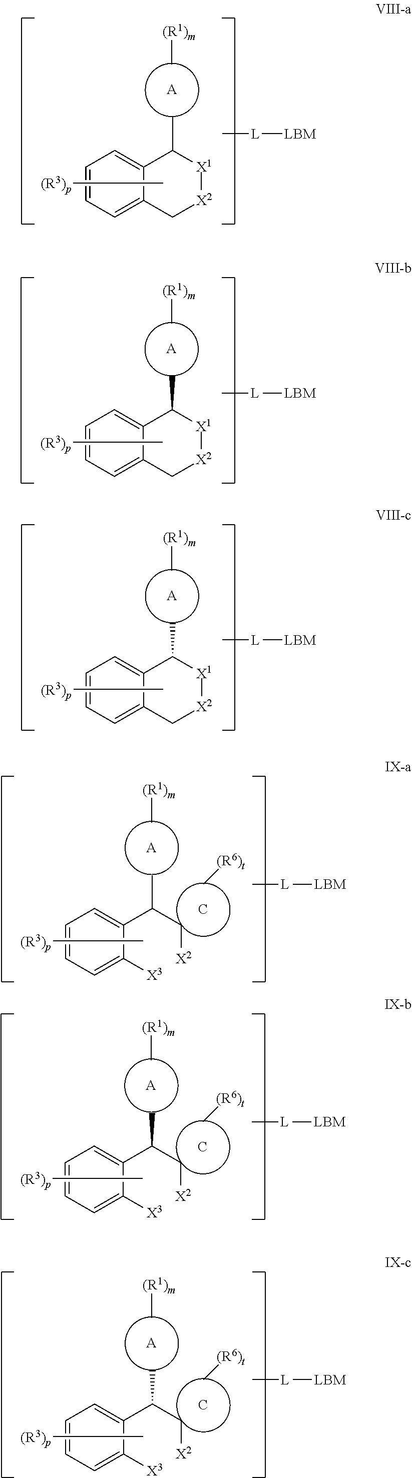

or a pharmaceutically acceptable salt thereof.

29. The compound of embodiment 28, wherein the compound is a compound of formula VIII-a, or a pharmaceutically acceptable salt thereof.

30. The compound of embodiment 28, wherein the compound is a compound of formula VIII-b, or a pharmaceutically acceptable salt thereof.

31. The compound of embodiment 28, wherein the compound is a compound of formula VIII-c, or a pharmaceutically acceptable salt thereof.

32. The compound of embodiment 28, wherein the compound is a compound of formula IX-a, or a pharmaceutically acceptable salt thereof.

33. The compound of embodiment 28, wherein the compound is a compound of formula IX-b, or a pharmaceutically acceptable salt thereof.

34. The compound of embodiment 28, wherein the compound is a compound of formula IX-e, or a pharmaceutically acceptable salt thereof.

35. The compound of any one of embodiments 1-18, wherein the compound is a compound of formula VIII-a-1, VIII-b-1, or VIII-c-1:

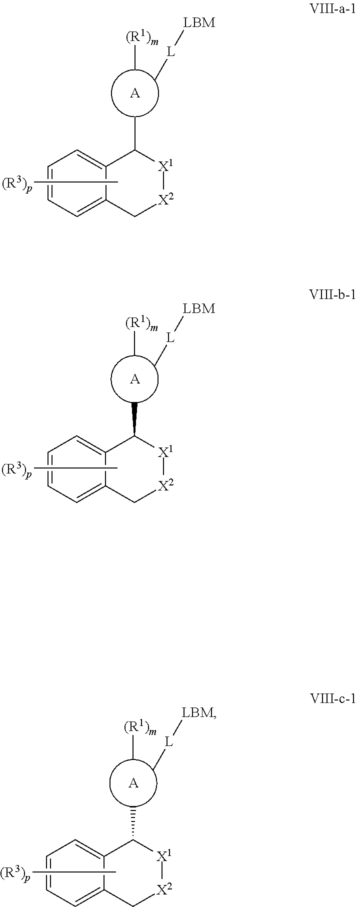

or a pharmaceutically acceptable salt thereof.

36. The compound of embodiment 35, wherein the compound is a compound of formula VIII-a-1, or a pharmaceutically acceptable salt thereof.

37. The compound of embodiment 35, wherein the compound is a compound of formula VIII-b-1, or a pharmaceutically acceptable salt thereof.

38. The compound of embodiment 35, wherein the compound is a compound of formula VIII-c-1, or a pharmaceutically acceptable salt thereof.

39. The compound of any one of embodiments 1-18, wherein the compound is a compound of formula IX-a-1, IX-b-1 or IX-c-1

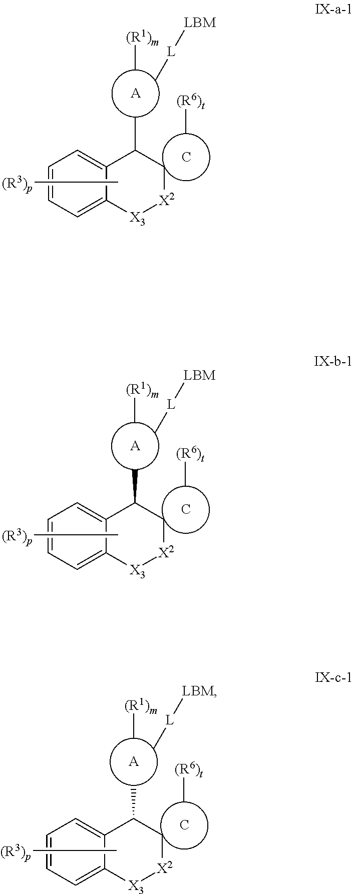

or a pharmaceutically acceptable salt thereof.

40. The compound of embodiment 39, wherein the compound is a compound of formula IX-a-1, or a pharmaceutically acceptable salt thereof.

41. The compound of embodiment 39, wherein the compound is a compound of formula IX-b-1, or a pharmaceutically acceptable salt thereof.

42. The compound of embodiment 39, wherein the compound is a compound of formula IX-c-1, or a pharmaceutically acceptable salt thereof.

43. The compound of any one of embodiments 1-42, wherein X¹ is N or CH.

44. The compound of any one of embodiments 1-42, wherein X¹ is N.

45. The compound of any one of embodiments 1-42, wherein X¹ is CH.

46. The compound of any one of embodiments 1-42, wherein X¹ is NH or CH₂.

47. The compound of any one of embodiments 1-42, wherein X¹ is CH(R^{A1}).

48. The compound of any one of embodiments 1-42, wherein X¹ is C(R^{A1}).

49. The compound of any one of embodiments 1-42, wherein X¹ is C(R^{A1})₂.

50. The compound of any one of embodiments 1-49, wherein X² is O or CH₂, provided that X¹ and X² are not both simultaneously heteroatoms.

51. The compound of any one of embodiments 1-49, wherein X² is O.

52. The compound of any one of embodiments 1-49, wherein X² is CH₂.

53. The compound of any one of embodiments 1-52, wherein X³ is O, CH₂, CH(R^{A5}) or C(R^{A5}).

54. The compound of any one of embodiments 1-52, wherein X³ is O.

55. The compound of any one of embodiments 1-52, wherein X³ is CH₂.

56. The compound of any one of embodiments 1-52, wherein X³ is CH(R^{A5}).

57. The compound of any one of embodiments 1-52, wherein X³ is C(R^{A5}).

58. The compound of any one of embodiments 1-18, wherein the compound is a compound of formula II-d, II-e, II-f, II-g, II-h, II-i, II-j, II-k, or II-l:

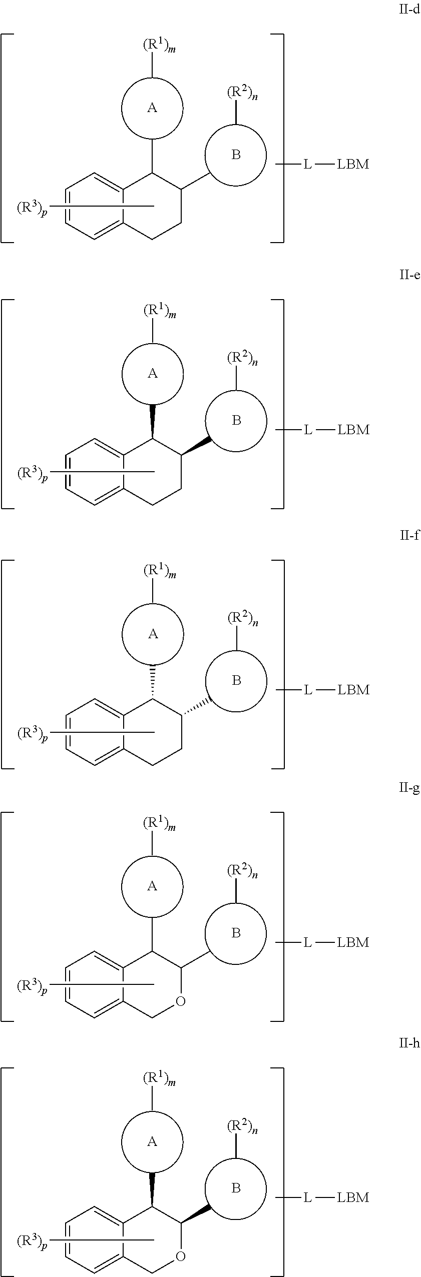

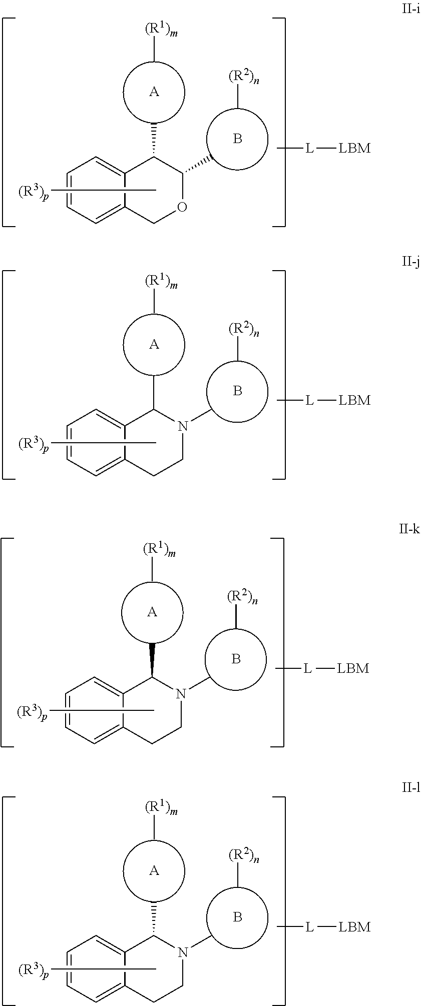

or a pharmaceutically acceptable salt thereof, provided that when the compound is of formula II-d, II-e, II-f, II-j, II-k and II-l and Ring B is phenyl, then Ring A is other than phenyl or a 6 membered monocyclic heteroaryl ring having 1-3 nitrogen heteroatoms.

59. The compound of embodiment 58, wherein the compound is a compound of formula II-d, or a pharmaceutically acceptable salt thereof, provided that when Ring B is phenyl, then Ring A is other than phenyl or a 6 membered monocyclic heteroaryl ring having 1-3 nitrogen heteroatoms.

60. The compound of embodiment 58, wherein the compound is a compound of formula II-e, or a pharmaceutically acceptable salt thereof, provided that when Ring B is phenyl, then Ring A is other than phenyl or a 6 membered monocyclic heteroaryl ring having 1-3 nitrogen heteroatoms.

61. The compound of embodiment 58, wherein the compound is a compound of formula II-f, or a pharmaceutically acceptable salt thereof, provided that when Ring B is phenyl, then Ring A is other than phenyl or a 6 membered monocyclic heteroaryl ring having 1-3 nitrogen heteroatoms.

62. The compound of embodiment 58, wherein the compound is a compound of formula II-g, or a pharmaceutically acceptable salt thereof.

63. The compound of embodiment 58, wherein the compound is a compound of formula II-h, or a pharmaceutically acceptable salt thereof.

64. The compound of embodiment 58, wherein the compound is a compound of formula II-i, or a pharmaceutically acceptable salt thereof.

65. The compound of embodiment 58, wherein the compound is a compound of formula II-j, or a pharmaceutically acceptable salt thereof, provided that when Ring B is phenyl, then Ring A is other than phenyl or a 6 membered monocyclic heteroaryl ring having 1-3 nitrogen heteroatoms.

66. The compound of embodiment 58, wherein the compound is a compound of formula II-k, or a pharmaceutically acceptable salt thereof, provided that when Ring B is phenyl, then Ring A is other than phenyl or a 6 membered monocyclic heteroaryl ring having 1-3 nitrogen heteroatoms.

67. The compound of embodiment 58, wherein the compound is a compound of formula II-l, or a pharmaceutically acceptable salt thereof, provided that when Ring B is phenyl, then Ring A is other than phenyl or a 6 membered monocyclic heteroaryl ring having 1-3 nitrogen heteroatoms.

68. The compound of any one of embodiments 1-18, wherein the compound is a compound of formula II-d-A, II-e-A, II-f-A, II-d-B, II-e-B, II-f-B, II-d-C, II-e-C, II-f-C, II-g, II-h, II-l, II-j-A, II-k-A, or II-l-A:

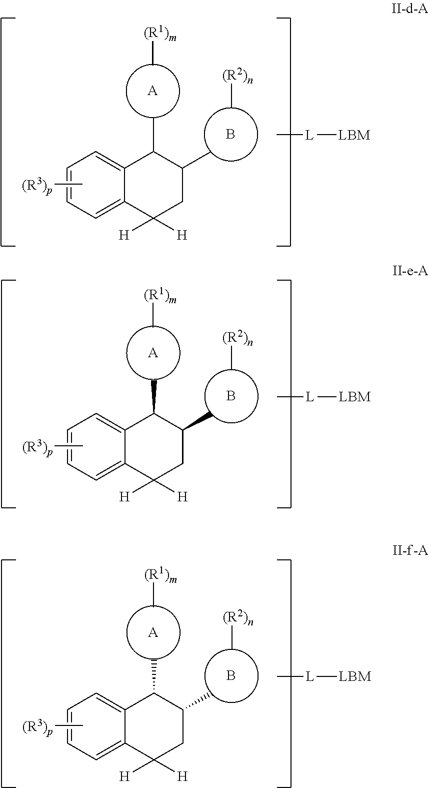

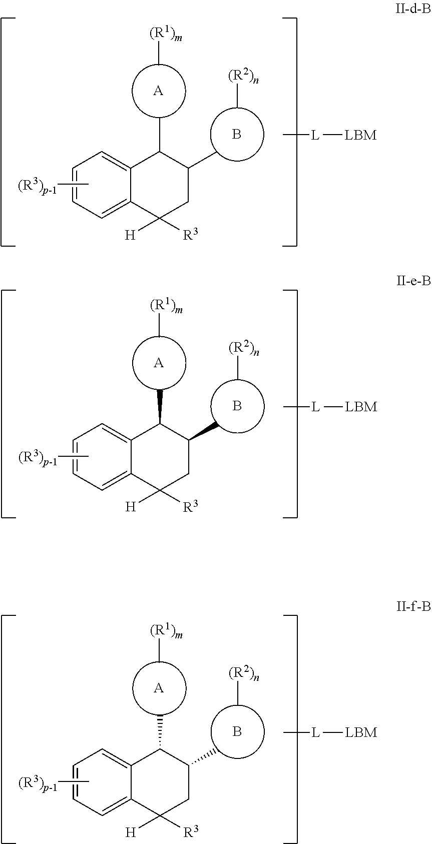

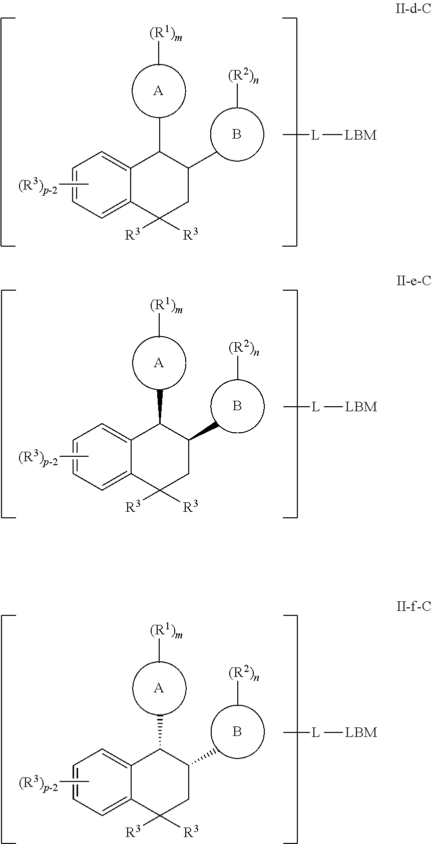

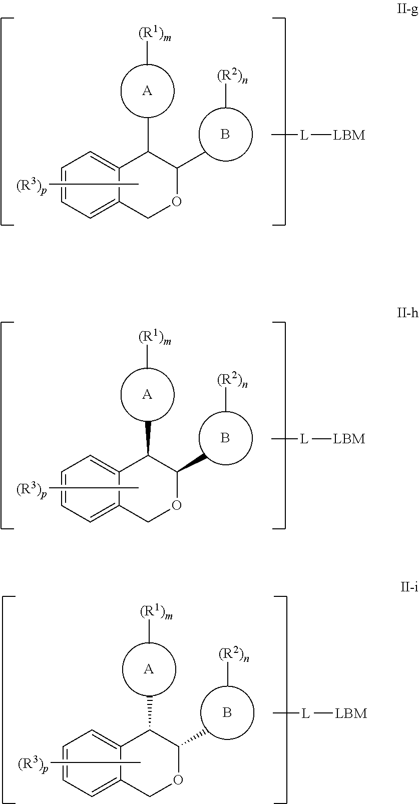

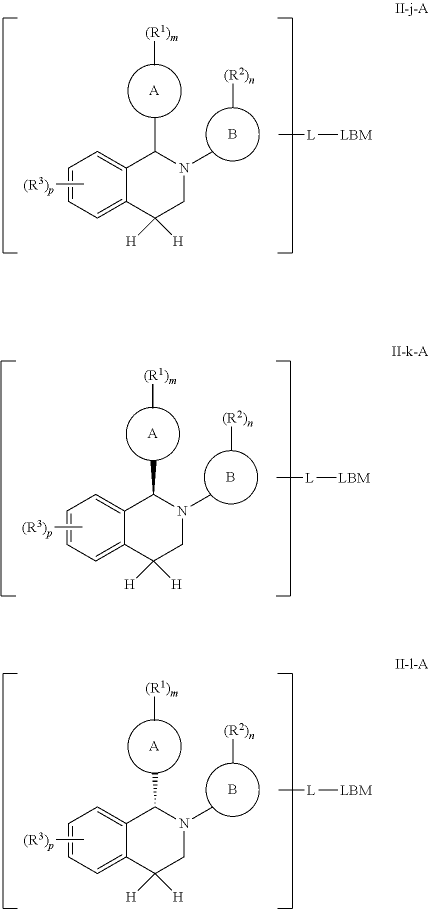

or a pharmaceutically acceptable salt thereof, provided that when the compound is of formula II-d-A, II-e-A, II-f-A, II-j-A, II-k-A and II-l-A and Ring B is phenyl, then Ring A is other than phenyl or a 6 membered monocyclic heteroaryl ring having 1-3 nitrogen heteroatoms.

69. The compound of embodiment 68, wherein the compound is a compound of formula II-d-A, or a pharmaceutically acceptable salt thereof, provided that when Ring B is phenyl, then Ring A is other than phenyl or a 6 membered monocyclic heteroaryl ring having 1-3 nitrogen heteroatoms.

70. The compound of embodiment 68, wherein the compound is a compound of formula II-e-A, or a pharmaceutically acceptable salt thereof, provided that when Ring B is phenyl, then Ring A is other than phenyl or a 6 membered monocyclic heteroaryl ring having 1-3 nitrogen heteroatoms.

71. The compound of embodiment 68, wherein the compound is a compound of formula II-f-A, or a pharmaceutically acceptable salt thereof, provided that when Ring B is phenyl, then Ring A is other than phenyl or a 6 membered monocyclic heteroaryl ring having 1-3 nitrogen heteroatoms.

72. The compound of embodiment 68, wherein the compound is a compound of formula II-d-B, or a pharmaceutically acceptable salt thereof.

73. The compound of embodiment 68, wherein the compound is a compound of formula II-e-B, or a pharmaceutically acceptable salt thereof.

74. The compound of embodiment 68, wherein the compound is a compound of formula II-f-B, or a pharmaceutically acceptable salt thereof.

75. The compound of embodiment 68, wherein the compound is a compound of formula II-d-C, or a pharmaceutically acceptable salt thereof.

76. The compound of embodiment 68, wherein the compound is a compound of formula II-e-C, or a pharmaceutically acceptable salt thereof.

77. The compound of embodiment 68, wherein the compound is a compound of formula II-f-C, or a pharmaceutically acceptable salt thereof.

78. The compound of embodiment 68, wherein the compound is a compound of formula II-j-A, or a pharmaceutically acceptable salt thereof, provided that when Ring B is phenyl, then Ring A is other than phenyl or a 6 membered monocyclic heteroaryl ring having 1-3 nitrogen heteroatoms.

79. The compound of embodiment 68, wherein the compound is a compound of formula II-k-A, or a pharmaceutically acceptable salt thereof, provided that when Ring B is phenyl, then Ring A is other than phenyl or a 6 membered monocyclic heteroaryl ring having 1-3 nitrogen heteroatoms.

80. The compound of embodiment 68, wherein the compound is a compound of formula II-l-A, or a pharmaceutically acceptable salt thereof, provided that when Ring B is phenyl, then Ring A is other than phenyl or a 6 membered monocyclic heteroaryl ring having 1-3 nitrogen heteroatoms.

81. The compound of any one of embodiments 1-18, wherein the compound is a compound of one of formulas II-n, II-o, II-p, II-q, II-r, II-s, II-t, II-u or II-v:

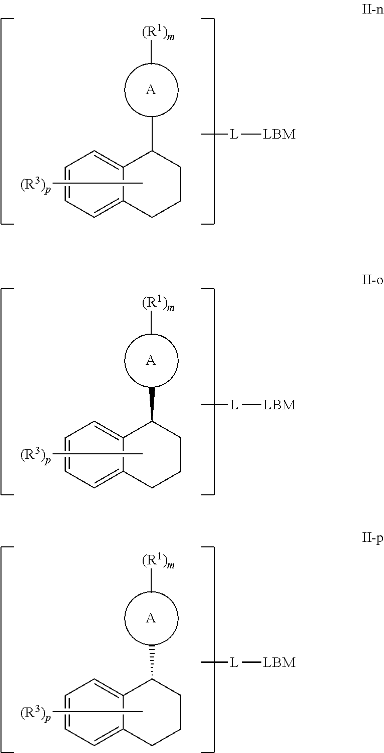

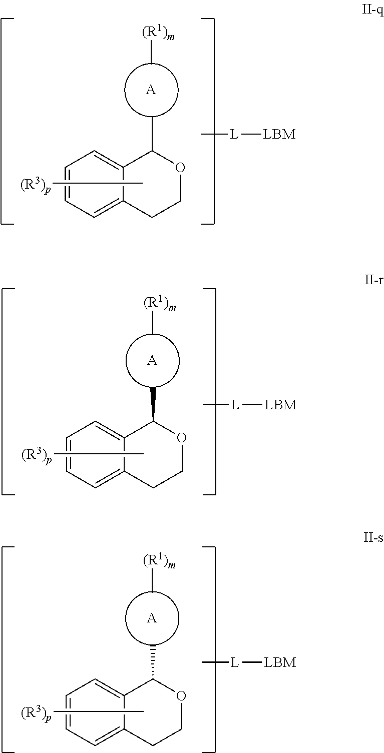

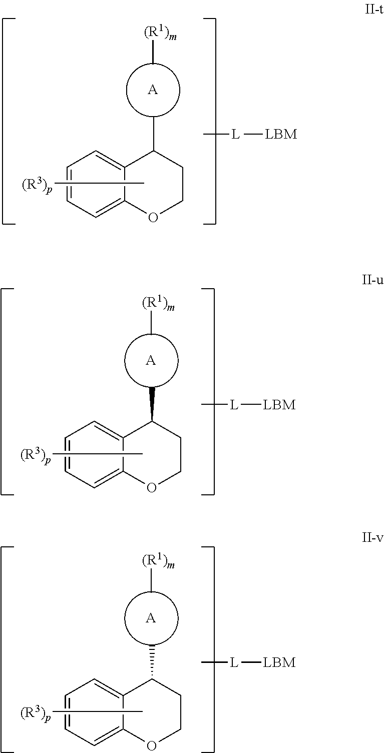

or a pharmaceutically acceptable salt thereof.

82. The compound of embodiment 81, wherein the compound is a compound of formula II-n, or a pharmaceutically acceptable salt thereof.

83. The compound of embodiment 81, wherein the compound is a compound of formula II-o, or a pharmaceutically acceptable salt thereof.

84. The compound of embodiment 81, wherein the compound is a compound of formula II-p, or a pharmaceutically acceptable salt thereof.

85. The compound of embodiment 81, wherein the compound is a compound of formula II-q, or a pharmaceutically acceptable salt thereof.

86. The compound of embodiment 81, wherein the compound is a compound of formula II-r, or a pharmaceutically acceptable salt thereof.

87. The compound of embodiment 81, wherein the compound is a compound of formula II-s, or a pharmaceutically acceptable salt thereof.

88. The compound of embodiment 81, wherein the compound is a compound of formula II-t, or a pharmaceutically acceptable salt thereof.

89. The compound of embodiment 81, wherein the compound is a compound of formula II-u, or a pharmaceutically acceptable salt thereof.

90. The compound of embodiment 81, wherein the compound is a compound of formula II-v, or a pharmaceutically acceptable salt thereof.

91. The compound of any one of embodiments 1-18, wherein the compound is a compound of one of formulas II-w, II-x, II-y, II-z, II-aa, II-bb, II-cc, II-dd or II-ee:

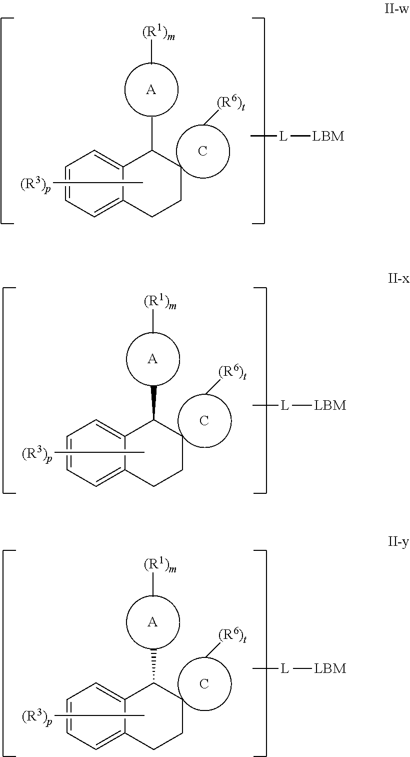

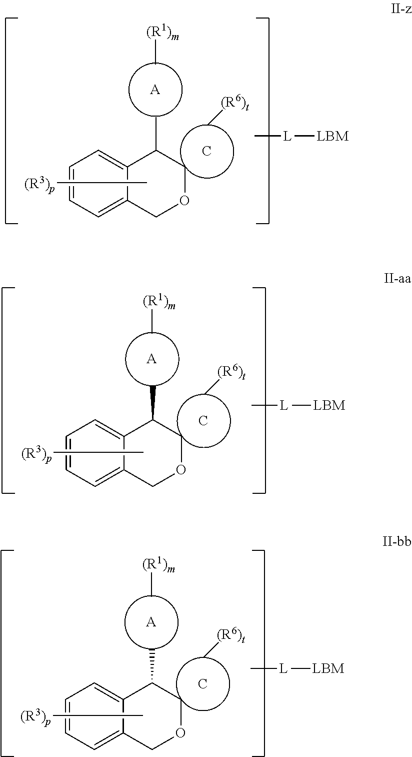

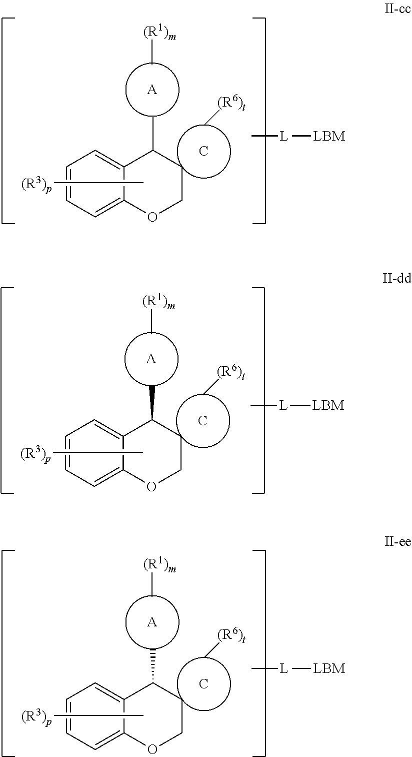

or a pharmaceutically acceptable salt thereof.

92. The compound of embodiment 91, wherein the compound is a compound of formula II-w, or a pharmaceutically acceptable salt thereof.

93. The compound of embodiment 91, wherein the compound is a compound of formula II-x, or a pharmaceutically acceptable salt thereof.

94. The compound of embodiment 91, wherein the compound is a compound of formula II-y, or a pharmaceutically acceptable salt thereof.

95. The compound of embodiment 91, wherein the compound is a compound of formula II-z, or a pharmaceutically acceptable salt thereof.

96. The compound of embodiment 91, wherein the compound is a compound of formula II-aa, or a pharmaceutically acceptable salt thereof.

97. The compound of embodiment 91, wherein the compound is a compound of formula II-bb, or a pharmaceutically acceptable salt thereof.

98. The compound of embodiment 91, wherein the compound is a compound of formula II-cc, or a pharmaceutically acceptable salt thereof.

99. The compound of embodiment 91, wherein the compound is a compound of formula II-dd, or a pharmaceutically acceptable salt thereof.

100. The compound of embodiment 91, wherein the compound is a compound of formula II-ee, or a pharmaceutically acceptable salt thereof.

101. The compound of any one of embodiments 1-18, wherein the compound is a compound of one of formulas II-d-1, II-e-1, II-f-1, II-g-1, II-h-1, II-i-1, II-j-1, II-k-1, or II-l-1;

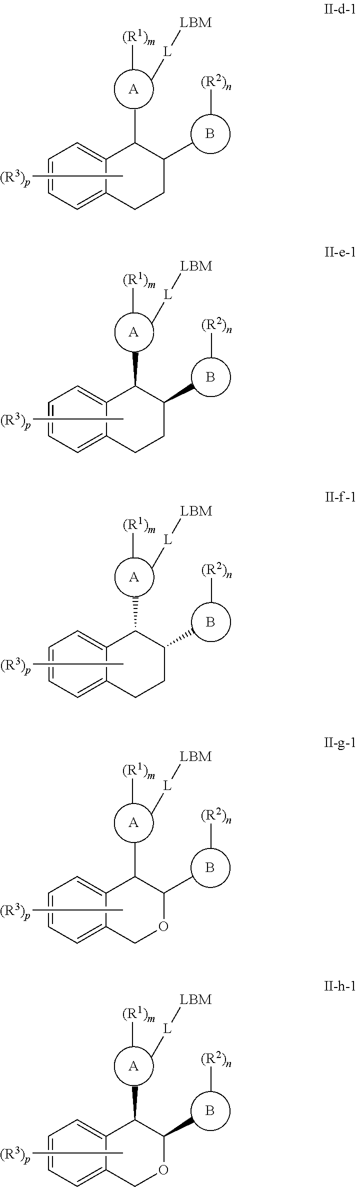

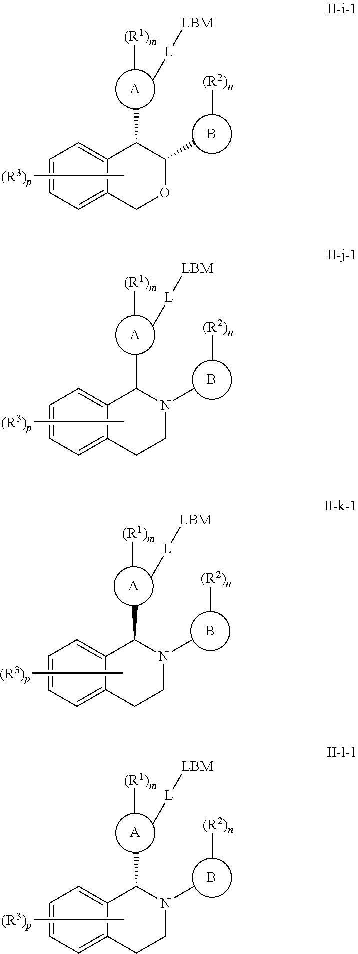

or a pharmaceutically acceptable salt thereof,
provided that when the compound is of formula II-d-1, II-e-1, II-f-1, II-j-1, II-k-1 and II-l-1 and Ring B is phenyl, then Ring A is other than phenyl or a 6 membered monocyclic heteroaryl ring having 1-3 nitrogen heteroatoms.

102. The compound of embodiment 101, wherein the compound is a compound of formula II-d-1, or a pharmaceutically acceptable salt thereof, provided that when Ring B is phenyl, then Ring A is other than phenyl or a 6 membered monocyclic heteroaryl ring having 1-3 nitrogen heteroatoms.

103. The compound of embodiment 101, wherein the compound is a compound of formula II-e-1, or a pharmaceutically acceptable salt thereof, provided that when Ring B is phenyl, then Ring A is other than phenyl or a 6 membered monocyclic heteroaryl ring having 1-3 nitrogen heteroatoms.

104. The compound of embodiment 101, wherein the compound is a compound of formula II-f-1, or a pharmaceutically acceptable salt thereof, provided that when Ring B is phenyl, then Ring A is other than phenyl or a 6 membered monocyclic heteroaryl ring having 1-3 nitrogen heteroatoms.

105. The compound of embodiment 101, wherein the compound is a compound of formula II-g-1, or a pharmaceutically acceptable salt thereof.

106. The compound of embodiment 101, wherein the compound is a compound of formula II-h-1, or a pharmaceutically acceptable salt thereof.

107. The compound of embodiment 101, wherein the compound is a compound of formula II-i-1, or a pharmaceutically acceptable salt thereof.

108. The compound of embodiment 101, wherein the compound is a compound of formula II-j-1, or a pharmaceutically acceptable salt thereof, provided that when Ring B is phenyl, then Ring A is other than phenyl or a 6 membered monocyclic heteroaryl ring having 1-3 nitrogen heteroatoms.

109. The compound of embodiment 101, wherein the compound is a compound of formula II-k-1, or a pharmaceutically acceptable salt thereof, provided that when Ring B is phenyl, then Ring A is other than phenyl or a 6 membered monocyclic heteroaryl ring having 1-3 nitrogen heteroatoms.

110. The compound of embodiment 101, wherein the compound is a compound of formula II-l-1, or a pharmaceutically acceptable salt thereof, provided that when Ring B is phenyl, then Ring A is other than phenyl or a 6 membered monocyclic heteroaryl ring having 1-3 nitrogen heteroatoms.

111. The compound of any one of embodiments 1-18, wherein the compound is a compound of formula II-d-1-A, II-e-1-A, II-f-1-A, II-d-1-B, II-e-1-B, II-f-1-B, II-d-1-C, II-e-1-C, II-f-1-C, II-j-1-A, II-k-1-A, or II-l-1-A:

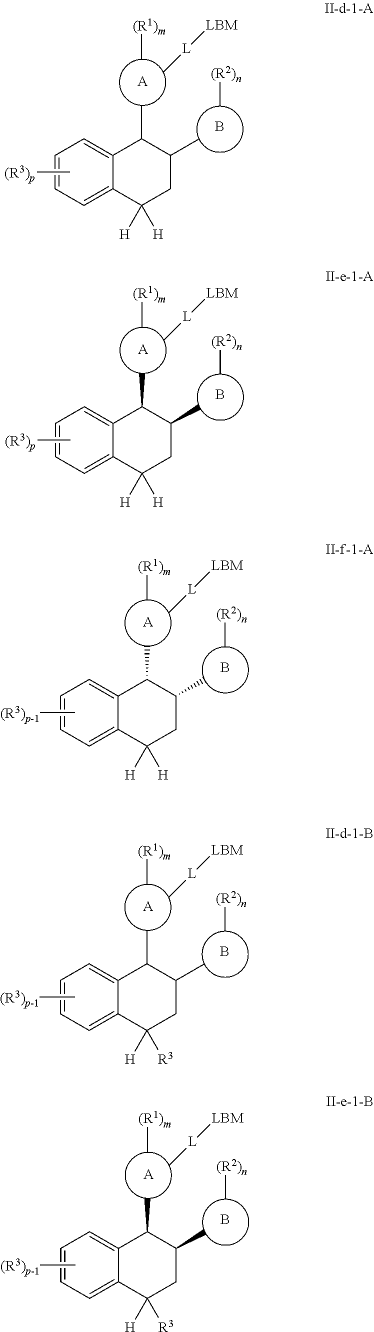

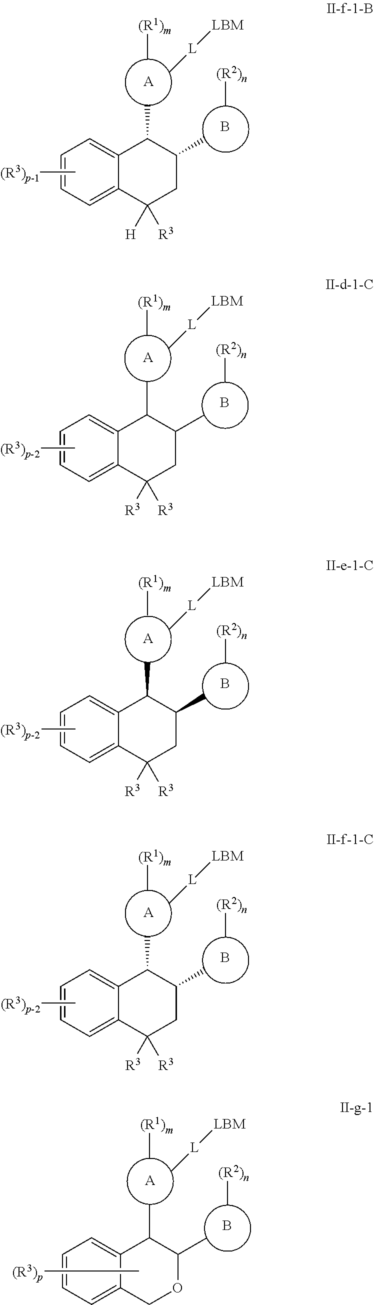

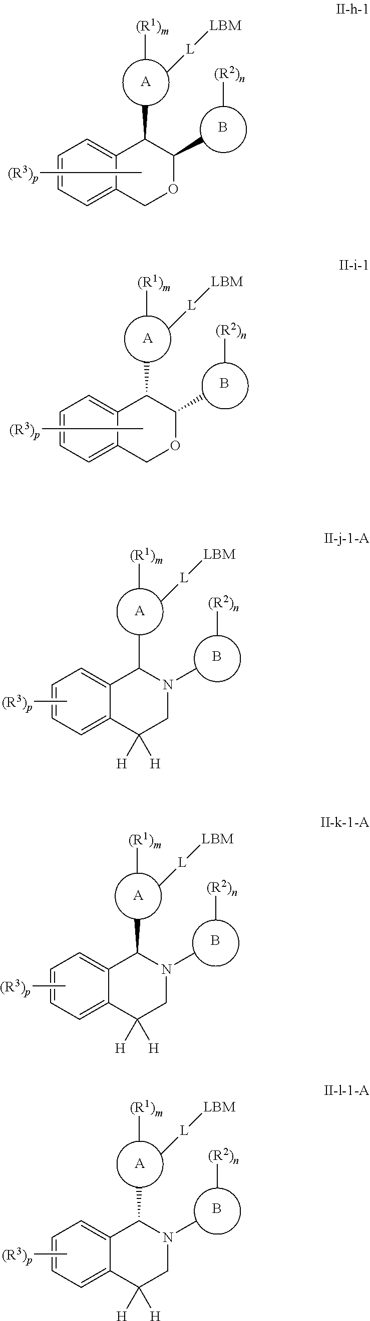

or a pharmaceutically acceptable salt thereof,
provided that when the compound is of formula II-d-1-A, II-e-1-A, II-f-1-A, II-j-1-A, II-k-1-A and II-l-1-A and Ring B is phenyl, then Ring A is other than phenyl or a 6 membered monocyclic heteroaryl ring having 1-3 nitrogen heteroatoms.

112. The compound of embodiment 111, wherein the compound is a compound of formula II-d-1-A, or a pharmaceutically acceptable salt thereof, provided that when Ring B is phenyl, then Ring A is other than phenyl or a 6 membered monocyclic heteroaryl ring having 1-3 nitrogen heteroatoms.

113. The compound of embodiment 111, wherein the compound is a compound of formula II-e-1-A, or a pharmaceutically acceptable salt thereof, provided that when Ring Bis phenyl, then Ring A is other than phenyl or a 6 membered monocyclic heteroaryl ring having 1-3 nitrogen heteroatoms.

114. The compound of embodiment 111, wherein the compound is a compound of formula II-f-1-A, or a pharmaceutically acceptable salt thereof, provided that when Ring B is phenyl, then Ring A is other than phenyl or a 6 membered monocyclic heteroaryl ring having 1-3 nitrogen heteroatoms.

115. The compound of embodiment 111, wherein the compound is a compound of formula II-d-1-B, or a pharmaceutically acceptable salt thereof.

116. The compound of embodiment 111, wherein the compound is a compound of formula II-e-1-B, or a pharmaceutically acceptable salt thereof.

117. The compound of embodiment 111, wherein the compound is a compound of formula II-f-1-B, or a pharmaceutically acceptable salt thereof.

118. The compound of embodiment 111, wherein the compound is a compound of formula II-d-1-C, or a pharmaceutically acceptable salt thereof.

119. The compound of embodiment 111, wherein the compound is a compound of formula II-e-1-C, or a pharmaceutically acceptable salt thereof.

120. The compound of embodiment 111, wherein the compound is a compound of formula II-f-1-C, or a pharmaceutically acceptable salt thereof.

121. The compound of embodiment 111, wherein the compound is a compound of formula II-j-1-A, or a pharmaceutically acceptable salt thereof, provided that when Ring B is phenyl, then Ring A is other than phenyl or a 6 membered monocyclic heteroaryl ring having 1-3 nitrogen heteroatoms.

122. The compound of embodiment 111, wherein the compound is a compound of formula II-k-1-A, or a pharmaceutically acceptable salt thereof, provided that when Ring B is phenyl, then Ring A is other than phenyl or a 6 membered monocyclic heteroaryl ring having 1-3 nitrogen heteroatoms.

123. The compound of embodiment 111, wherein the compound is a compound of formula II-l-1-A, or a pharmaceutically acceptable salt thereof, provided that when Ring Bis phenyl, then Ring A is other than phenyl or a 6 membered monocyclic heteroaryl ring having 1-3 nitrogen heteroatoms.

124. The compound of any one of embodiments 1-18, wherein the compound is a compound of formula II-m-1:

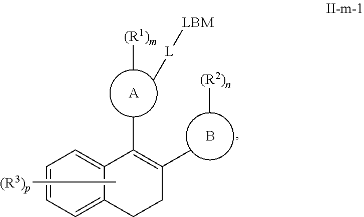

or a pharmaceutically acceptable salt thereof.

125. The compound of any one of embodiments 1-18, wherein the compound is a compound of one of formulas II-n-1, II-o-1, II-p-1, II-q-1, II-r-1, II-s-1, II-t-1, II-u-1 or II-v-1:

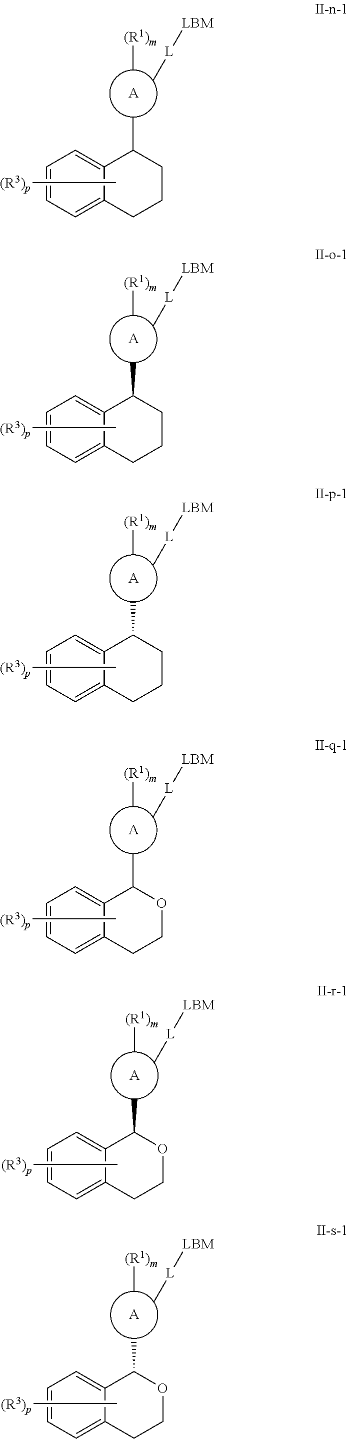

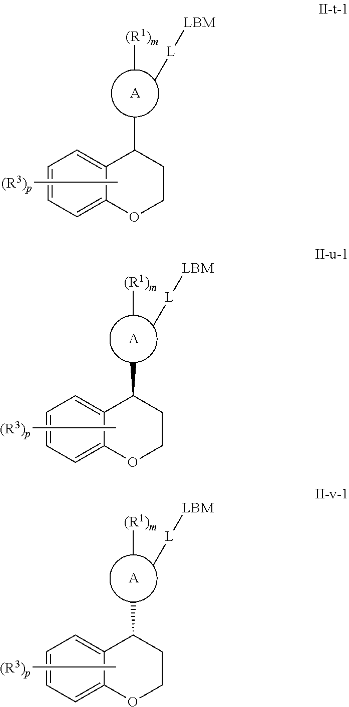

or a pharmaceutically acceptable salt thereof.

126. The compound of embodiment 125, wherein the compound is a compound of formula II-n-1, or a pharmaceutically acceptable salt thereof.

127. The compound of embodiment 125, wherein the compound is a compound of formula II-o-1, or a pharmaceutically acceptable salt thereof.

128. The compound of embodiment 125, wherein the compound is a compound of formula II-p-1, or a pharmaceutically acceptable salt thereof.

129. The compound of embodiment 125, wherein the compound is a compound of formula II-q-1, or a pharmaceutically acceptable salt thereof.

130. The compound of embodiment 125, wherein the compound is a compound of formula II-r-1, or a pharmaceutically acceptable salt thereof.

131. The compound of embodiment 125, wherein the compound is a compound of formula II-s-1, or a pharmaceutically acceptable salt thereof.

132. The compound of embodiment 125, wherein the compound is a compound of formula II-t-1, or a pharmaceutically acceptable salt thereof.

133. The compound of embodiment 125, wherein the compound is a compound of formula II-u-1, or a pharmaceutically acceptable salt thereof.

134. The compound of embodiment 125, wherein the compound is a compound of formula II-v-1, or a pharmaceutically acceptable salt thereof.

135. The compound of any one of embodiments 1-18, wherein the compound is a compound of one of formulas II-w-1, II-x-1, II-y-1, II-z-1, II-aa-1, II-bb-1, II-cc-1, II-dd-1 or II-ee-1:

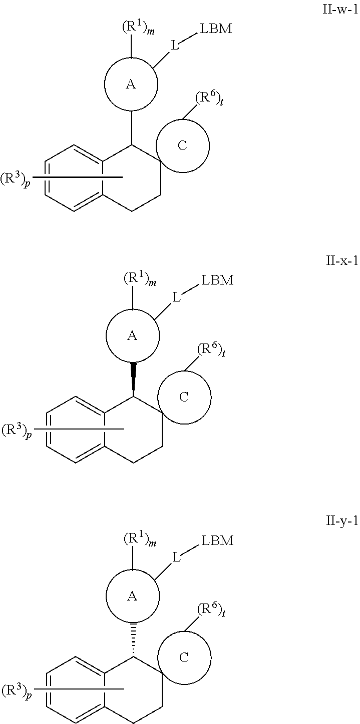

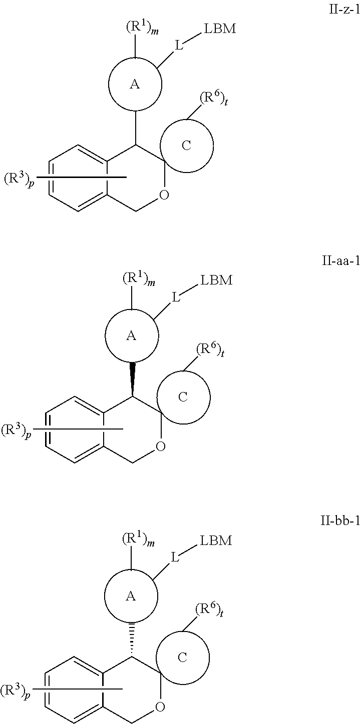

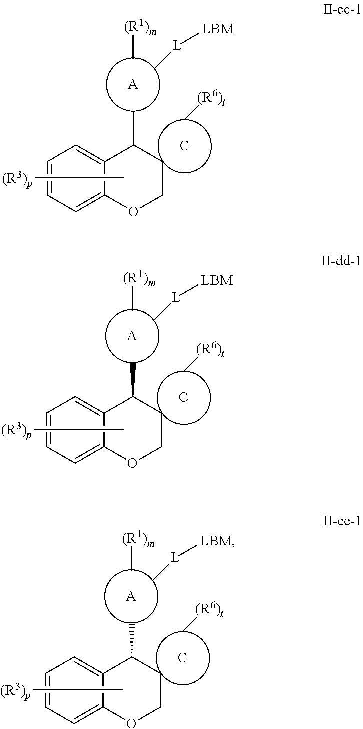

or a pharmaceutically acceptable salt thereof.

136. The compound of embodiment 135, wherein the compound is a compound of formula II-w-1, or a pharmaceutically acceptable salt thereof.

137. The compound of embodiment 135, wherein the compound is a compound of formula II-x-1, or a pharmaceutically acceptable salt thereof.

138. The compound of embodiment 135, wherein the compound is a compound of formula II-y-1, or a pharmaceutically acceptable salt thereof.

139. The compound of embodiment 135, wherein the compound is a compound of formula II-z-1, or a pharmaceutically acceptable salt thereof.

140. The compound of embodiment 135, wherein the compound is a compound of formula II-aa-1, or a pharmaceutically acceptable salt thereof.

141. The compound of embodiment 135, wherein the compound is a compound of formula II-bb-1, or a pharmaceutically acceptable salt thereof.

142. The compound of embodiment 135, wherein the compound is a compound of formula II-cc-1, or a pharmaceutically acceptable salt thereof.

143. The compound of embodiment 135, wherein the compound is a compound of formula II-dd-1, or a pharmaceutically acceptable salt thereof.

144. The compound of embodiment 135, wherein the compound is a compound of formula II-ee-1, or a pharmaceutically acceptable salt thereof.

145. The compound of any one of embodiments 1-18, wherein the compound is a compound of one of formulas II-ff-1, II-gg-1, II-hh-1, II-ii-1, II-jj-1, II-kk-1, II-ll-1, II-mm-1 or II-nn-1:

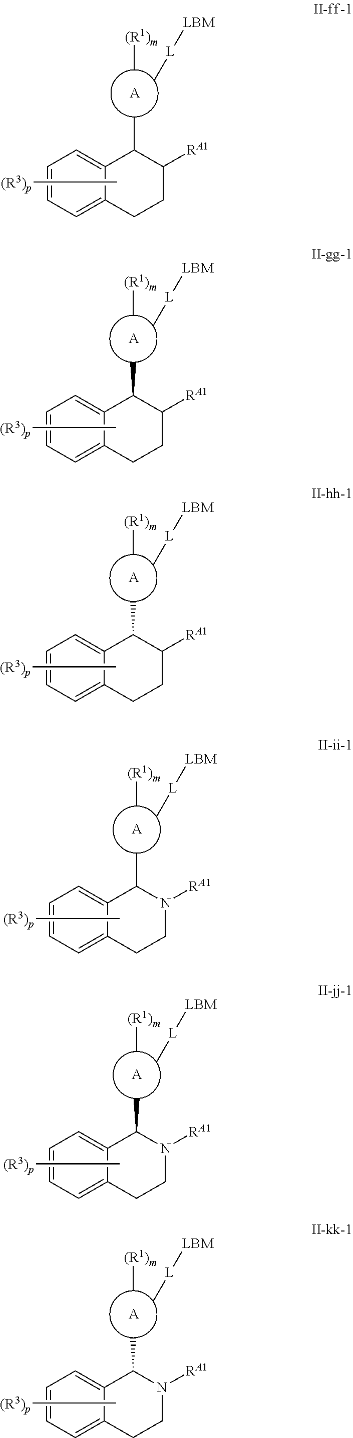

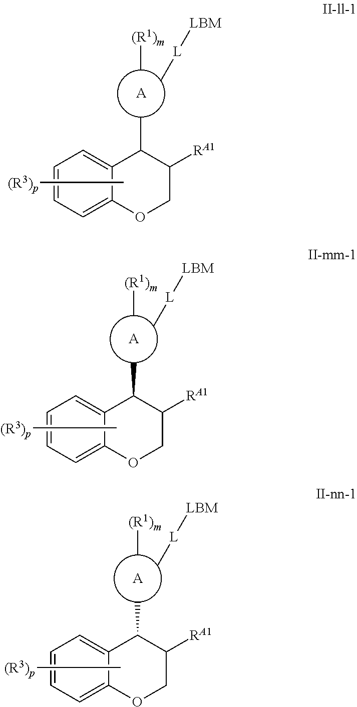

or a pharmaceutically acceptable salt thereof.

146. The compound of embodiment 145, wherein the compound is a compound of formula II-ff-1, or a pharmaceutically acceptable salt thereof.

147. The compound of embodiment 145, wherein the compound is a compound of formula II-gg-1, or a pharmaceutically acceptable salt thereof.

148. The compound of embodiment 145, wherein the compound is a compound of formula II-hh-1, or a pharmaceutically acceptable salt thereof.

149. The compound of embodiment 145, wherein the compound is a compound of formula II-ii-1, or a pharmaceutically acceptable salt thereof.

150. The compound of embodiment 145, wherein the compound is a compound of formula II-jj-1, or a pharmaceutically acceptable salt thereof.

151. The compound of embodiment 145, wherein the compound is a compound of formula II-kk-1, or a pharmaceutically acceptable salt thereof.

152. The compound of embodiment 145, wherein the compound is a compound of formula II-ll-1, or a pharmaceutically acceptable salt thereof.

153. The compound of embodiment 145, wherein the compound is a compound of formula II-mm-1, or a pharmaceutically acceptable salt thereof.

154. The compound of embodiment 145, wherein the compound is a compound of formula II-nn-1, or a pharmaceutically acceptable salt thereof.

155. The compound of any one of embodiments 1-154, wherein Ring A together with its R¹ substituents is selected from

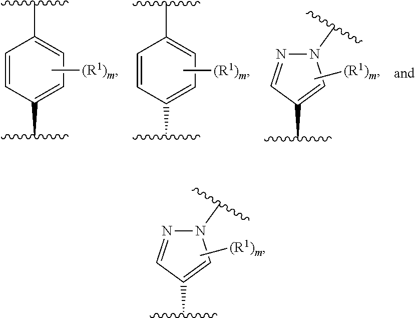

wherein the top attachment point connects to L and the bottom attachment point connects to the six-member ring of the ERBM moiety.

156. The compound of any one of embodiments 1-154, wherein Ring A together with its R¹ substituents is selected from

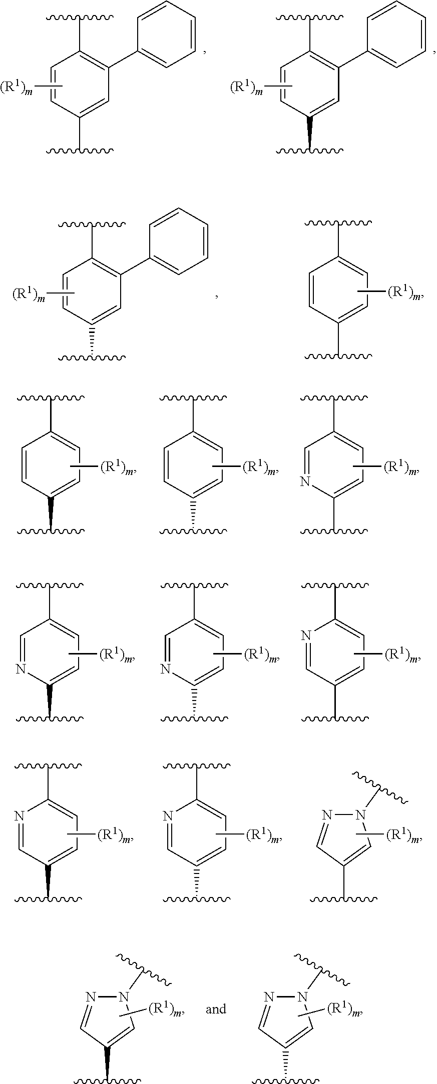

wherein the top attachment point connects to L and the bottom attachment point connects to the six-member ring of the ERBM moiety.

157. The compound of any one of embodiments 1-154, wherein Ring A together with its R¹ substituents is

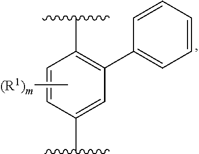

wherein the top attachment point connects to L and the bottom attachment point connects to the six-member ring of the ERBM moiety.

158. The compound of any one of embodiments 1-154, wherein Ring A together with its R¹ substituents is

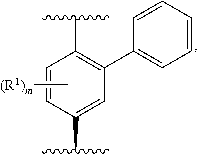

wherein the top attachment point connects to L and the bottom attachment point connects to the six-member ring of the ERBM moiety.

159. The compound of any one of embodiments 1-154, wherein Ring A together with its R¹ substituents is

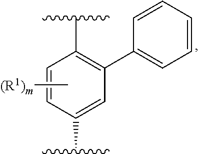

wherein the top attachment point connects to L and the bottom attachment point connects to the six-member ring of the ERBM moiety.

160. The compound of any one of embodiments 1-154, wherein Ring A together with its R¹ substituents is

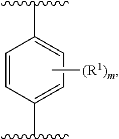

wherein the top attachment point connects to L and the bottom attachment point connects to the six-member ring of the ERBM moiety.

161. The compound of any one of embodiments 1-154, wherein Ring A together with its R¹ substituents is

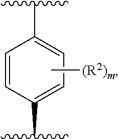

wherein the top attachment point connects to L and the bottom attachment point connects to the six-member ring of the ERBM moiety.

162. The compound of any one of embodiments 1-154, wherein Ring A together with its R¹ substituents is

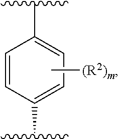

wherein the top attachment point connects to L and the bottom attachment point connects to the six-member ring of the ERBM moiety.

163. The compound of any one of embodiments 1-154, wherein Ring A together with its R¹ substituents is

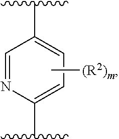

wherein the top attachment point connects to L and the bottom attachment point connects to the six-member ring of the ERBM moiety.

164. The compound of any one of embodiments 1-154, wherein Ring A together with its R¹ substituents is

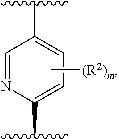

wherein the top attachment point connects to L and the bottom attachment point connects to the six-member ring of the ERBM moiety.

165. The compound of any one of embodiments 1-154, wherein Ring A together with its R¹ substituents is

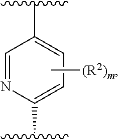

wherein the top attachment point connects to L and the bottom attachment point connects to the six-member ring of the ERBM moiety.

166. The compound of any one of embodiments 1-154, wherein Ring A together with its R¹ substituents is

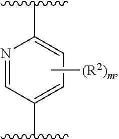

wherein the top attachment point connects to L and the bottom attachment point connects to the six-member ring of the ERBM moiety.

167. The compound of any one of embodiments 1-154, wherein Ring A together with its R¹ substituents is

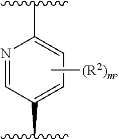

wherein the top attachment point connects to L and the bottom attachment point connects to the six-member ring of the ERBM moiety.

168. The compound of any one of embodiments 1-154, wherein Ring A together with its R¹ substituents is

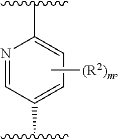

wherein the top attachment point connects to L and the bottom attachment point connects to the six-member ring of the ERBM moiety.

169. The compound of any one of embodiments 1-154, wherein Ring A together with its R¹ substituents is

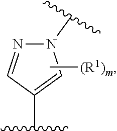

wherein the top attachment point connects to L and the bottom attachment point connects to the six-member ring of the ERBM moiety.

170. The compound of any one of embodiments 1-154, wherein Ring A together with its R¹ substituents is

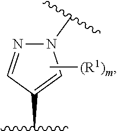

wherein the top attachment point connects to L and the bottom attachment point connects to the six-member ring of the ERBM moiety.

171. The compound of any one of embodiments 1-154, wherein Ring A together with its R¹ substituents is

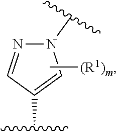

wherein the top attachment point connects to L and the bottom attachment point connects to the six-member ring of the ERBM moiety.

172. The compound of any one of embodiments 1-171, wherein Ring B together with its R² substituents is selected from

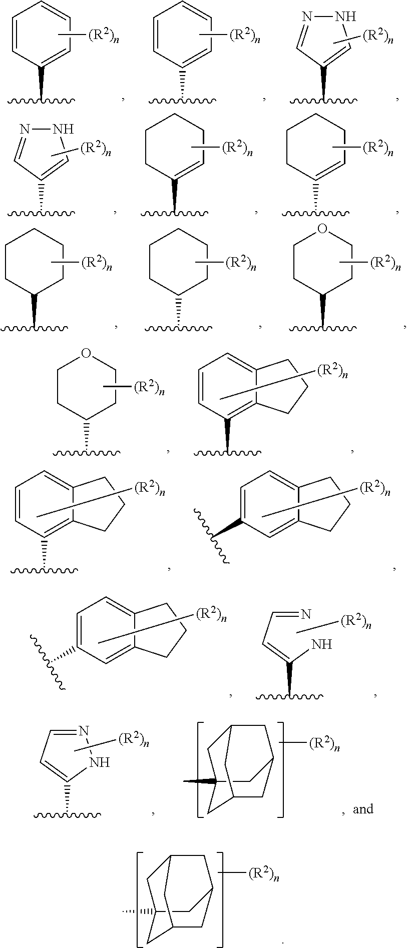

173. The compound of any one of embodiments 1-171, wherein Ring B together with its R² substituents is selected from

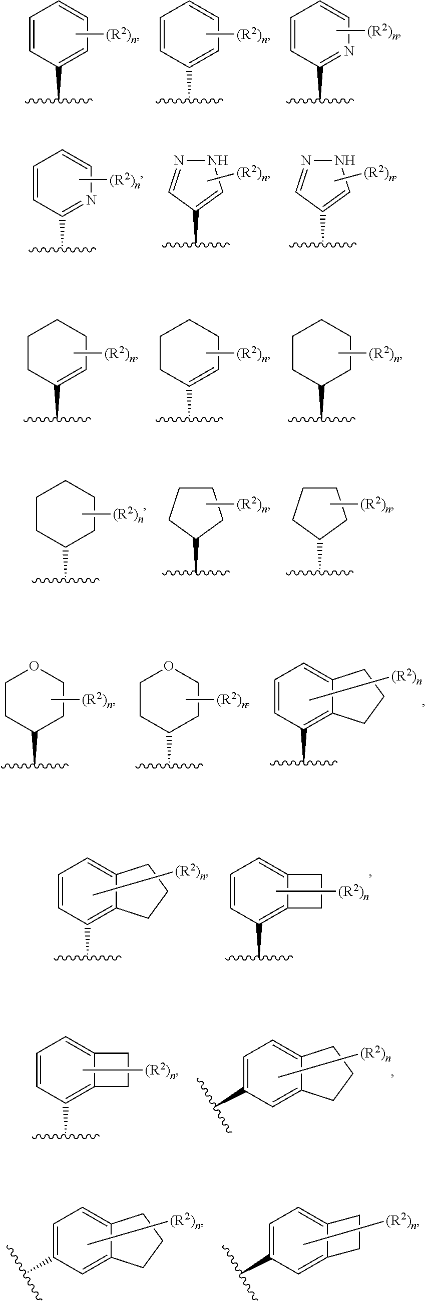

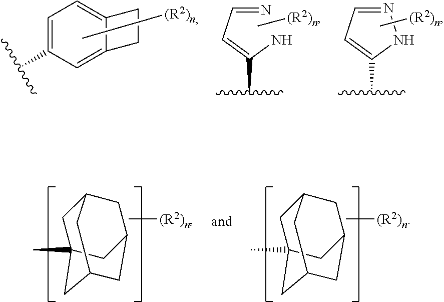

174. The compound of any one of embodiments 1-171, wherein Ring B together with its R² substituents is selected from:

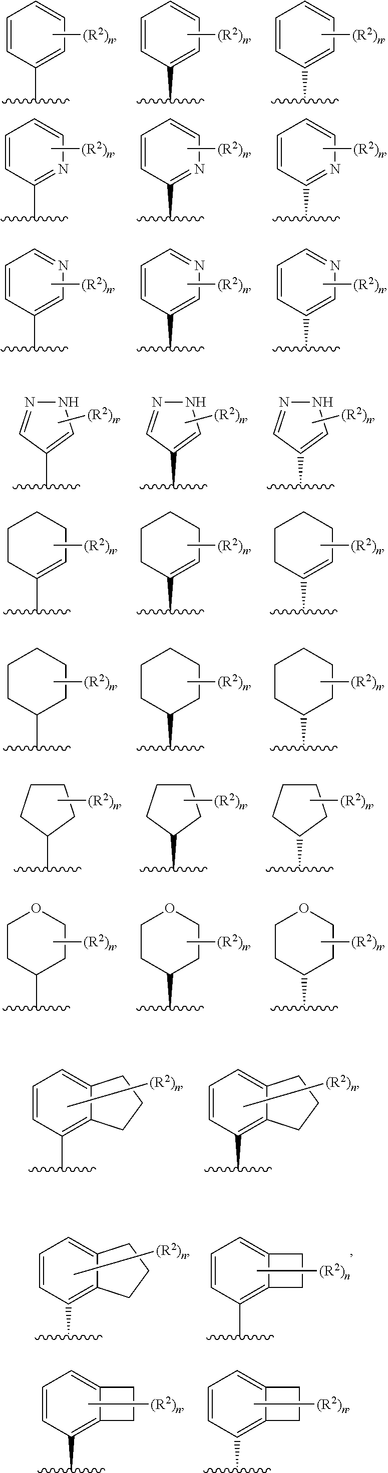

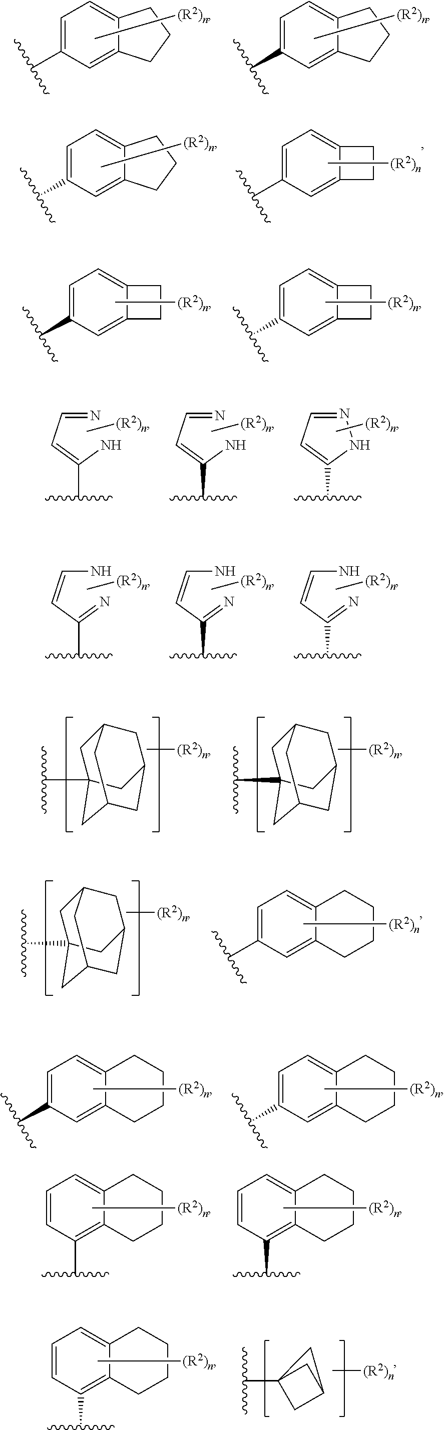

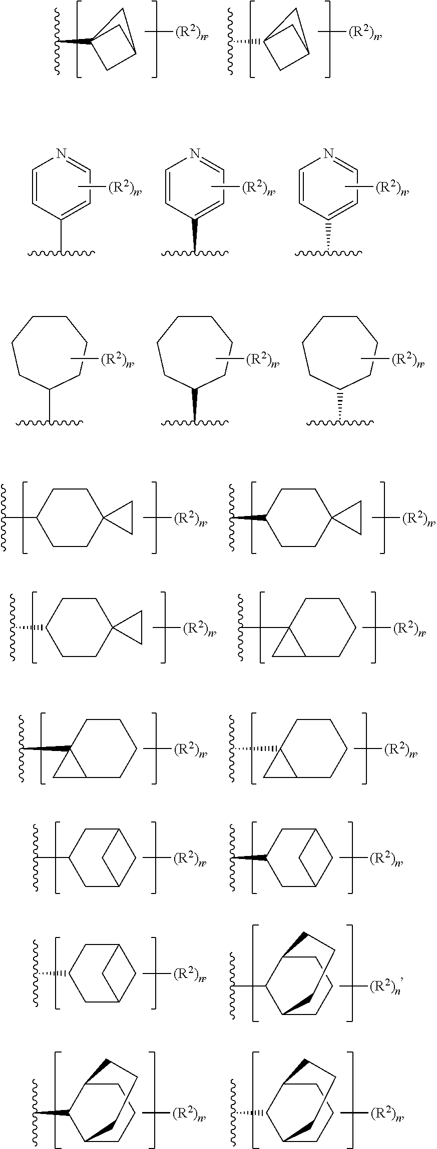

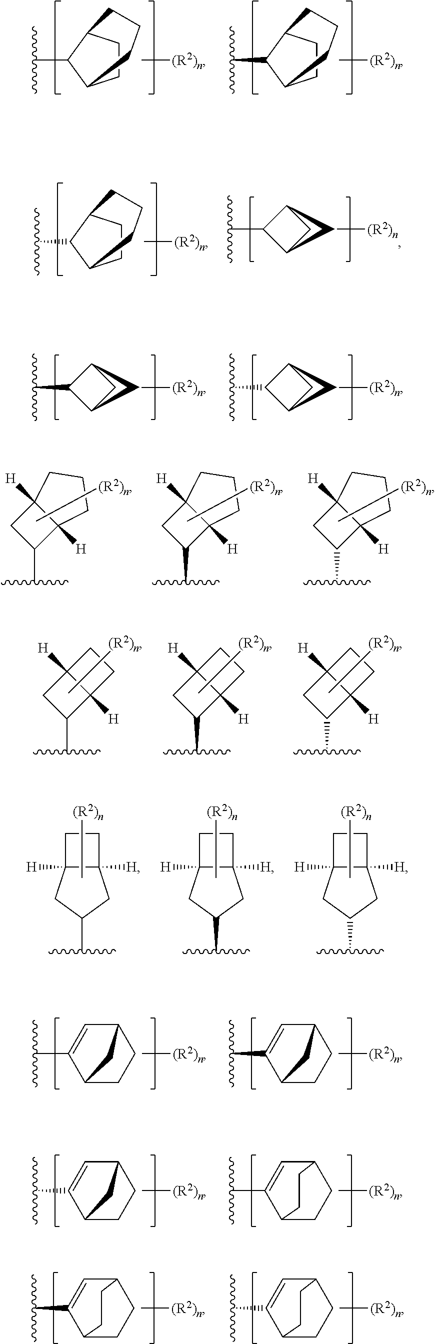

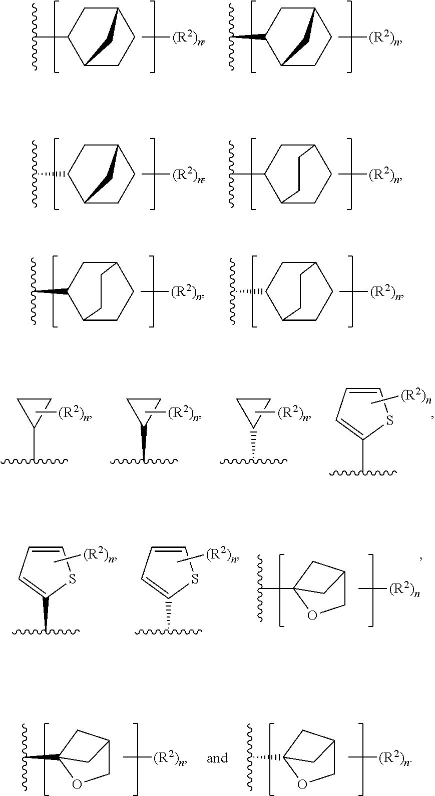

175. The compound of any one of embodiments 1-171, wherein Ring B together with its R² substituents is selected from:

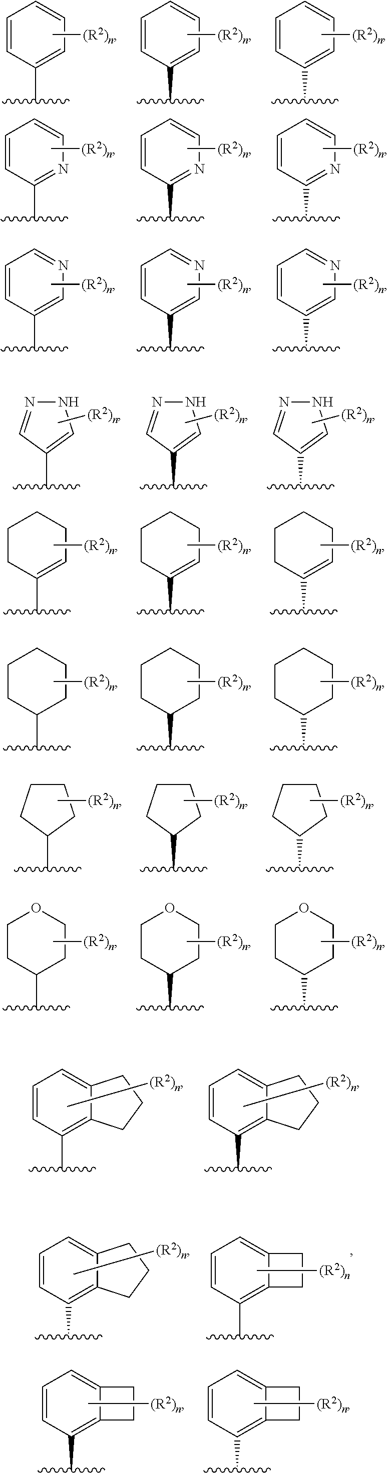

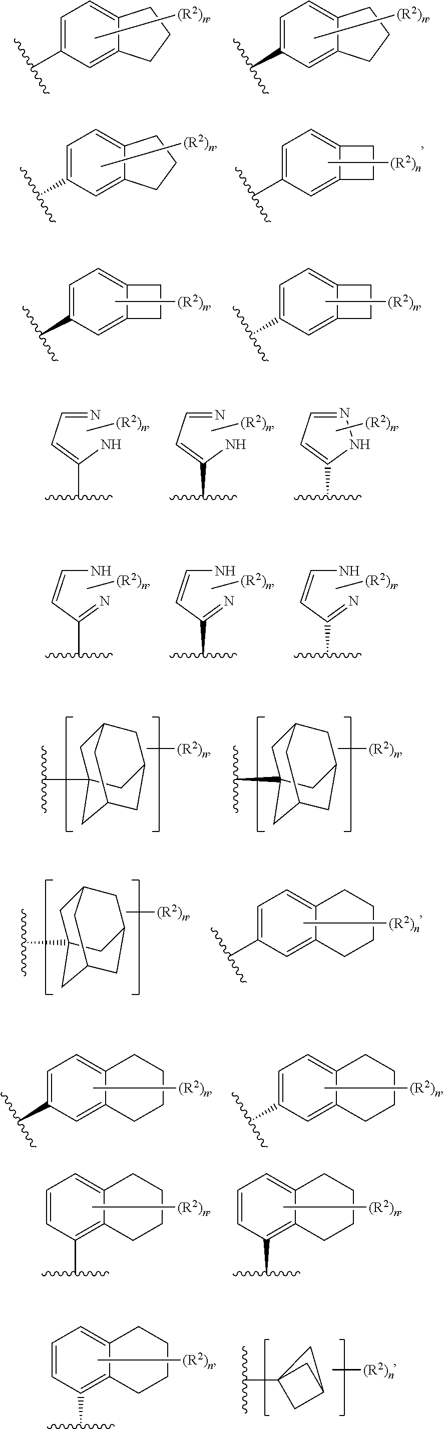

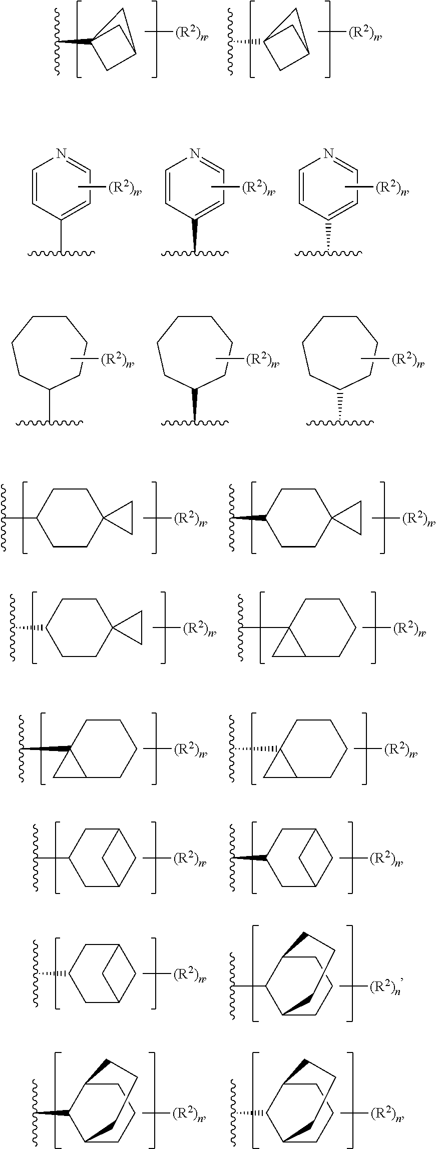

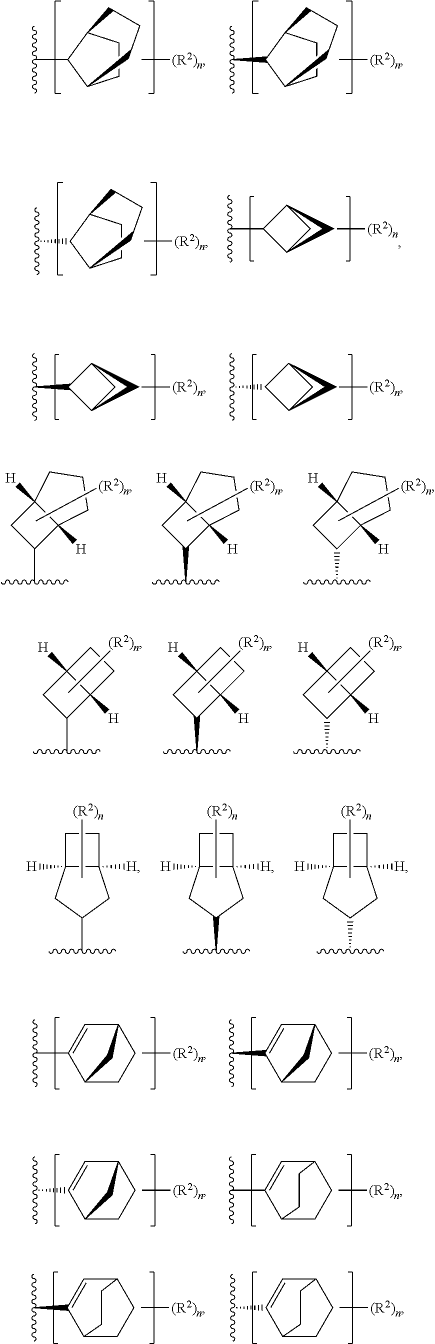

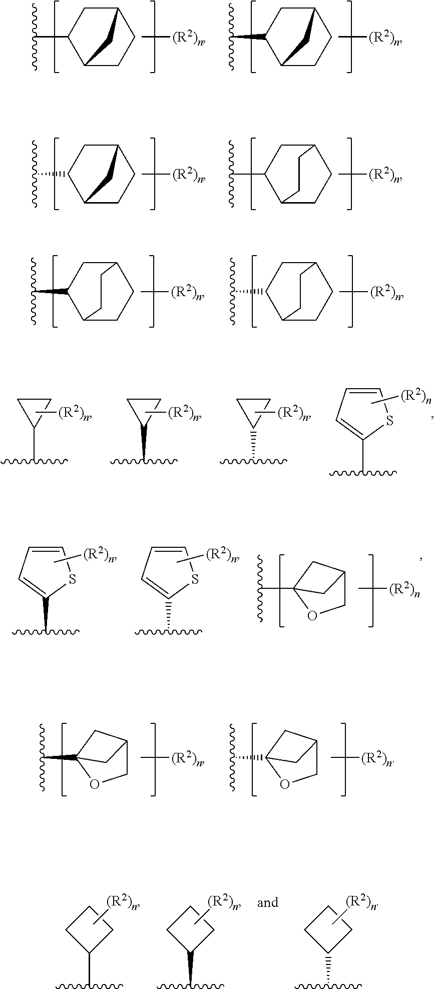

176. The compound of any one of embodiments 1-171, wherein Ring B together with its R² substituents is selected from

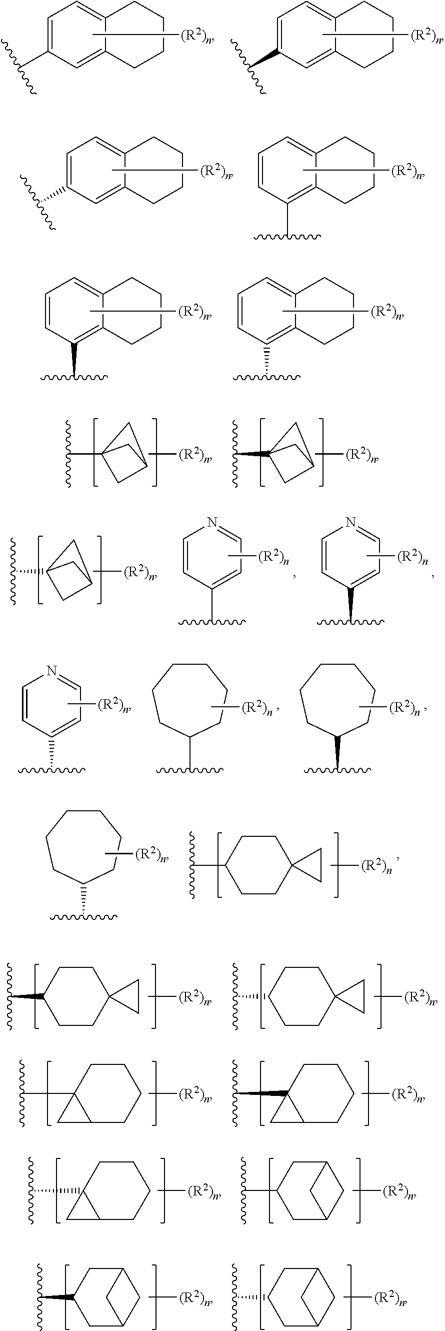

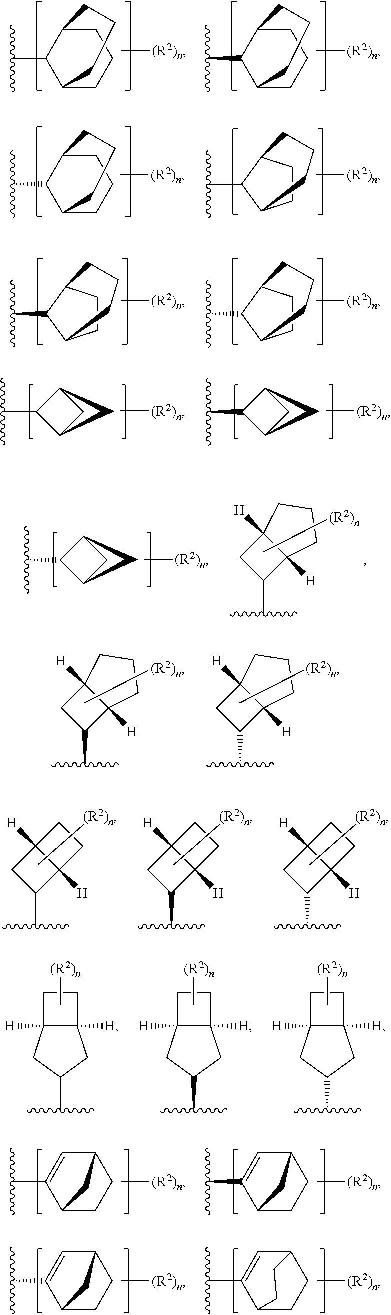

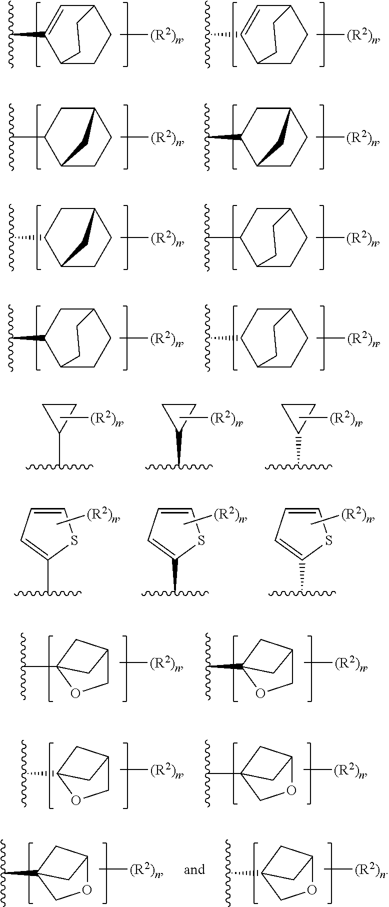

177. The compound of any one of embodiments 1-171, wherein Ring B together with its R² substituents is

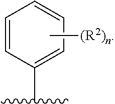

178. The compound of any one of embodiments 1-171, wherein Ring B together with its R² substituents is

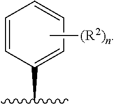

179. The compound of any one of embodiments 1-171, wherein Ring B together with its R² substituents is

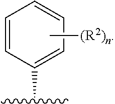

180. The compound of any one of embodiments 1-171, wherein Ring B together with its R² substituents is

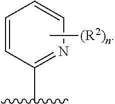

181. The compound of any one of embodiments 1-171, wherein Ring B together with its R² substituents is

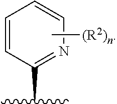

182. The compound of any one of embodiments 1-171, wherein Ring B together with its R² substituents is

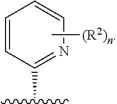

183. The compound of any one of embodiments 1-171, wherein Ring B together with its R² substituents is

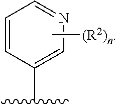

184. The compound of any one of embodiments 1-171, wherein Ring B together with its R² substituents is

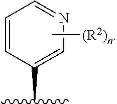

185. The compound of any one of embodiments 1-171, wherein Ring B together with its R² substituents is

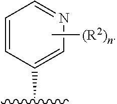

186. The compound of any one of embodiments 1-171, wherein Ring B together with its R² substituents is

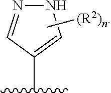

187. The compound of any one of embodiments 1-171, wherein Ring B together with its R² substituents is

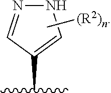

188. The compound of any one of embodiments 1-171, wherein Ring B together with its R² substituents is

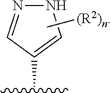

189. The compound of any one of embodiments 1-171, wherein Ring B together with its R² substituents is

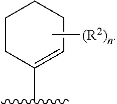

190. The compound of any one of embodiments 1-171, wherein Ring B together with its R² substituents is

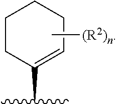

191. The compound of any one of embodiments 1-171, wherein Ring B together with its R² substituents is

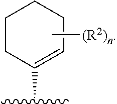

192. The compound of any one of embodiments 1-171, wherein Ring B together with its R² substituents is

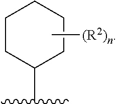

193. The compound of any one of embodiments 1-171, wherein Ring B together with its R² substituents is

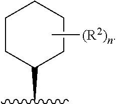

194. The compound of any one of embodiments 1-171, wherein Ring B together with its R² substituents is

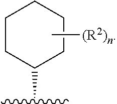

195. The compound of any one of embodiments 1-171, wherein Ring B together with its R² substituents is

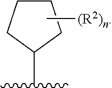

196. The compound of any one of embodiments 1-171, wherein Ring B together with its R² substituents is

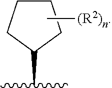

197. The compound of any one of embodiments 1-171, wherein Ring B together with its R² substituents is

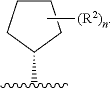

198. The compound of any one of embodiments 1-171, wherein Ring B together with its R² substituents is

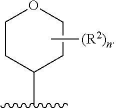

199. The compound of any one of embodiments 1-171, wherein Ring B together with its R² substituents is

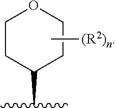

200. The compound of any one of embodiments 1-171, wherein Ring B together with its R² substituents is

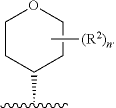

201. The compound of any one of embodiments 1-171, wherein Ring B together with its R² substituents is

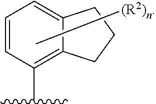

202. The compound of any one of embodiments 1-171, wherein Ring B together with its R² substituents is

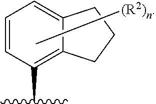

203. The compound of any one of embodiments 1-171, wherein Ring B together with its R² substituents is

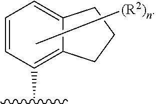

204. The compound of any one of embodiments 1-171, wherein Ring B together with its R² substituents is

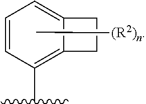

205. The compound of any one of embodiments 1-171, wherein Ring B together with its R² substituents is

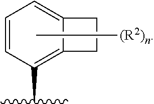

206. The compound of any one of embodiments 1-171, wherein Ring B together with its R² substituents is

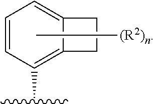

207. The compound of any one of embodiments 1-171, wherein Ring B together with its R² substituents is

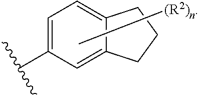

208. The compound of any one of embodiments 1-171, wherein Ring B together with its R² substituents is

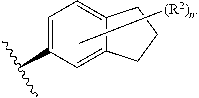

209. The compound of any one of embodiments 1-171, wherein Ring B together with its R² substituents is

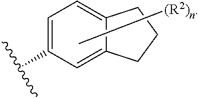

210. The compound of any one of embodiments 1-171, wherein Ring B together with its R² substituents is

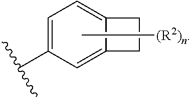

211. The compound of any one of embodiments 1-171, wherein Ring B together with its R² substituents is

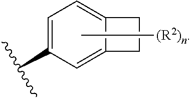

212. The compound of any one of embodiments 1-171, wherein Ring B together with its R² substituents is

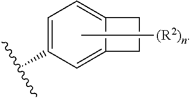

213. The compound of any one of embodiments 1-171, wherein Ring B together with its R² substituents is

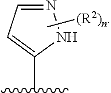

214. The compound of any one of embodiments 1-171, wherein Ring B together with its R² substituents is

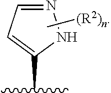

215. The compound of any one of embodiments 1-171, wherein Ring B together with its R² substituents is

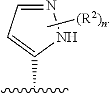

216. The compound of any one of embodiments 1-171, wherein Ring B together with its R² substituents is

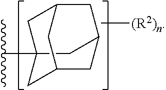

217. The compound of any one of embodiments 1-171, wherein Ring B together with its R² substituents is

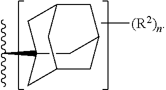

218. The compound of any one of embodiments 1-171, wherein Ring B together with its R² substituents is

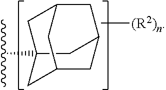

219. The compound of any one of embodiments 1-171, wherein Ring B together with its R² substituents is

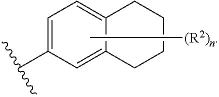

220. The compound of any one of embodiments 1-171, wherein Ring B together with its R² substituents is

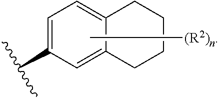

221. The compound of any one of embodiments 1-171, wherein Ring B together with its R² substituents is

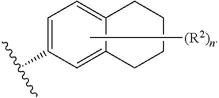

222. The compound of any one of embodiments 1-171, wherein Ring B together with its R² substituents is

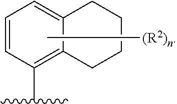

223. The compound of any one of embodiments 1-171, wherein Ring B together with its R² substituents is

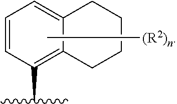

224. The compound of any one of embodiments 1-171, wherein Ring B together with its R² substituents is

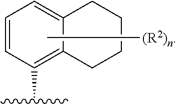

225. The compound of any one of embodiments 1-171, wherein Ring B together with its R² substituents is

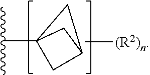

226. The compound of any one of embodiments 1-171, wherein Ring B together with its R² substituents is

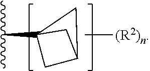

227. The compound of any one of embodiments 1-171, wherein Ring B together with its R² substituents is

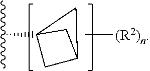

228. The compound of any one of embodiments 1-171, wherein Ring B together with its R² substituents is

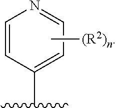

229. The compound of any one of embodiments 1-171, wherein Ring B together with its R² substituents is

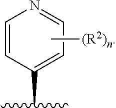

230. The compound of any one of embodiments 1-171, wherein Ring B together with its R² substituents is

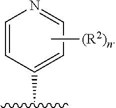

231. The compound of any one of embodiments 1-171, wherein Ring B together with its R² substituents is

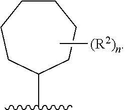

232. The compound of any one of embodiments 1-171, wherein Ring B together with its R² substituents is

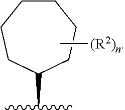

233. The compound of any one of embodiments 1-171, wherein Ring B together with its R² substituents is

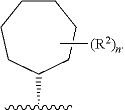

234. The compound of any one of embodiments 1-171, wherein Ring B together with its R² substituents is

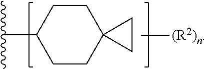

235. The compound of any one of embodiments 1-171, wherein Ring B together with its R² substituents is

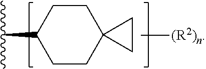

236. The compound of any one of embodiments 1-171, wherein Ring B together with its R² substituents is

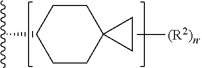

237. The compound of any one of embodiments 1-171, wherein Ring B together with its R² substituents is

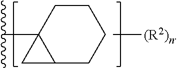

238. The compound of any one of embodiments 1-171, wherein Ring B together with its R² substituents is

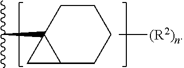

239. The compound of any one of embodiments 1-171, wherein Ring B together with its R² substituents is

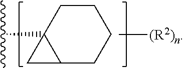

240. The compound of any one of embodiments 1-171, wherein Ring B together with its R² substituents is

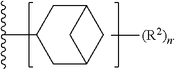

241. The compound of any one of embodiments 1-171, wherein Ring B together with its R² substituents is

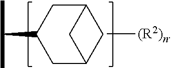

242. The compound of any one of embodiments 1-171, wherein Ring B together with its R² substituents is

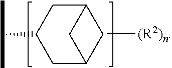

243. The compound of any one of embodiments 1-171, wherein Ring B together with its R² substituents is

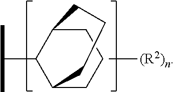

244. The compound of any one of embodiments 1-171, wherein Ring B together with its R² substituents is

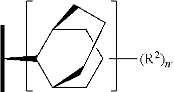

245. The compound of any one of embodiments 1-171, wherein Ring B together with its R² substituents is

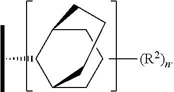

246. The compound of any one of embodiments 1-171, wherein Ring B together with its R² substituents is

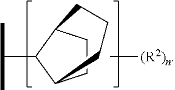

247. The compound of any one of embodiments 1-171, wherein Ring B together with its R² substituents is

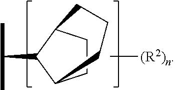

248. The compound of any one of embodiments 1-171, wherein Ring B together with its R² substituents is

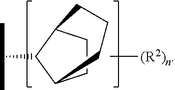

249. The compound of any one of embodiments 1-171, wherein Ring B together with its R² substituents is

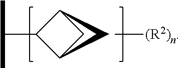

250. The compound of any one of embodiments 1-171, wherein Ring B together with its R² substituents is

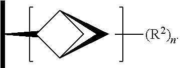

251. The compound of any one of embodiments 1-171, wherein Ring B together with its R² substituents is

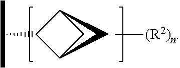

252. The compound of any one of embodiments 1-171, wherein Ring B together with its R² substituents is

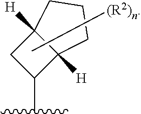

253. The compound of any one of embodiments 1-171, wherein Ring B together with its R² substituents is

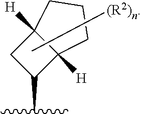

254. The compound of any one of embodiments 1-171, wherein Ring B together with its R² substituents is

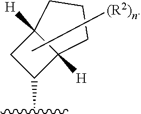

255. The compound of any one of embodiments 1-171, wherein Ring B together with its R² substituents is

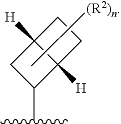

256. The compound of any one of embodiments 1-171, wherein Ring B together with its R² substituents is

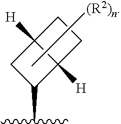

257. The compound of any one of embodiments 1-171, wherein Ring B together with its R² substituents is

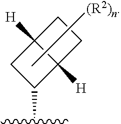

258. The compound of any one of embodiments 1-171, wherein Ring B together with its R² substituents is

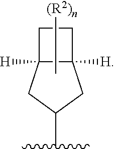

259. The compound of any one of embodiments 1-171, wherein Ring B together with its R² substituents is

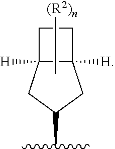

260. The compound of any one of embodiments 1-171, wherein Ring B together with its R² substituents is

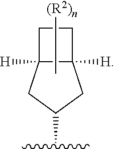

261. The compound of any one of embodiments 1-171, wherein Ring B together with its R² substituents is

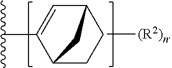

262. The compound of any one of embodiments 1-171, wherein Ring B together with its R² substituents is

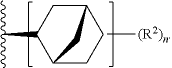

263. The compound of any one of embodiments 1-171, wherein Ring B together with its R² substituents is

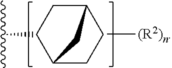

264. The compound of any one of embodiments 1-171, wherein Ring B together with its R² substituents is

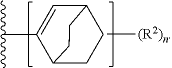

265. The compound of any one of embodiments 1-171, wherein Ring B together with its R² substituents is

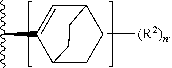

266. The compound of any one of embodiments 1-171, wherein Ring B together with its R² substituents is

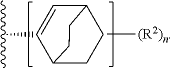

267. The compound of any one of embodiments 1-171, wherein Ring B together with its R² substituents is

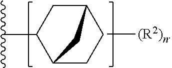

268. The compound of any one of embodiments 1-171, wherein Ring B together with its R² substituents is

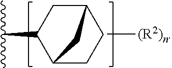

269. The compound of any one of embodiments 1-171, wherein Ring B together with its R² substituents is

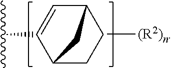

270. The compound of any one of embodiments 1-171, wherein Ring B together with its R² substituents is

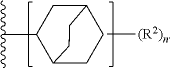

271. The compound of any one of embodiments 1-171, wherein Ring B together with its R² substituents is

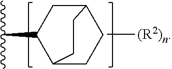

272. The compound of any one of embodiments 1-171, wherein Ring B together with its R² substituents is

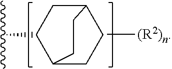

273. The compound of any one of embodiments 1-171, wherein Ring B together with its R² substituents is

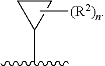

274. The compound of any one of embodiments 1-171, wherein Ring B together with its R² substituents is

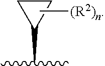

275. The compound of any one of embodiments 1-171, wherein Ring B together with its R² substituents is

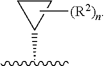

276. The compound of any one of embodiments 1-171, wherein Ring B together with its R² substituents is

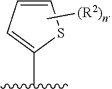

277. The compound of any one of embodiments 1-171, wherein Ring B together with its R² substituents is

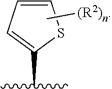

278. The compound of any one of embodiments 1-171, wherein Ring B together with its R² substituents is

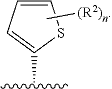

279. The compound of any one of embodiments 1-171, wherein Ring B together with its R² substituents is

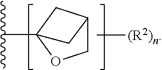

280. The compound of any one of embodiments 1-171, wherein Ring B together with its R² substituents is

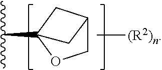

281. The compound of any one of embodiments 1-171, wherein Ring B together with its R² substituents is

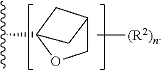

282. The compound of any one of embodiments 1-171, wherein Ring B together with its R² substituents is

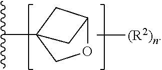

283. The compound of any one of embodiments 1-171, wherein Ring B together with its R² substituents is

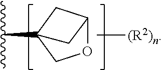

284. The compound of any one of embodiments 1-171, wherein Ring B together with its R² substituents is

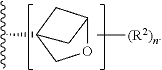

285. The compound of any one of embodiments 1-171, wherein Ring B together with its R² substituents is

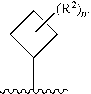

286. The compound of any one of embodiments 1-171, wherein Ring B together with its R² substituents is

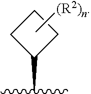

287. The compound of any one of embodiments 1-171, wherein Ring B together with its R² substituents is

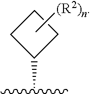

288. The compound of any one of embodiments 1-287, wherein the compound is a compound of one of formulas III-a, III-b, or III-c:

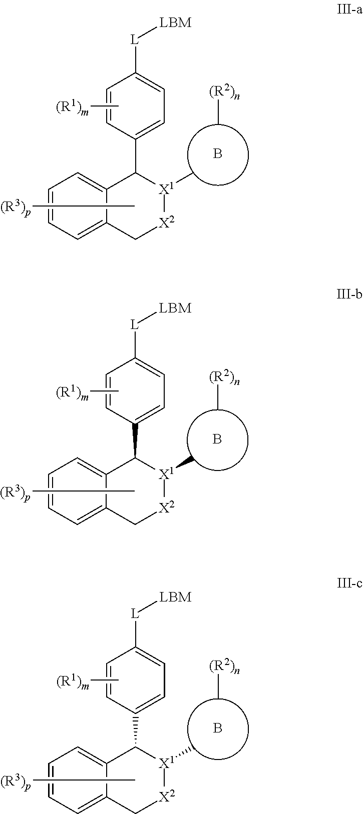

or a pharmaceutically acceptable salt thereof, provided that when X¹ is CH or N and X² is CH₂, then Ring B is other than phenyl.

289. The compound of embodiment 288, wherein the compound is a compound of formula III-a, or a pharmaceutically acceptable salt thereof, provided that when X¹ is CH or N and X² is CH₂, then Ring B is other than phenyl.

290. The compound of embodiment 288, wherein the compound is a compound of formula III-b, or a pharmaceutically acceptable salt thereof, provided that when X¹ is CH or N and X² is CH₂, then Ring B is other than phenyl.

291. The compound of embodiment 288, wherein the compound is a compound of formula III-c, or a pharmaceutically acceptable salt thereof, provided that when X¹ is CH or N and X² is CH₂, then Ring B is other than phenyl.

292. The compound of any one of embodiments 1-287, wherein the compound is a compound of one of formulas III-a-A, III-b-A, III-c-A, III-a-B, III-b-B, III-c-B, III-a-C, III-b-C and III-c-C:

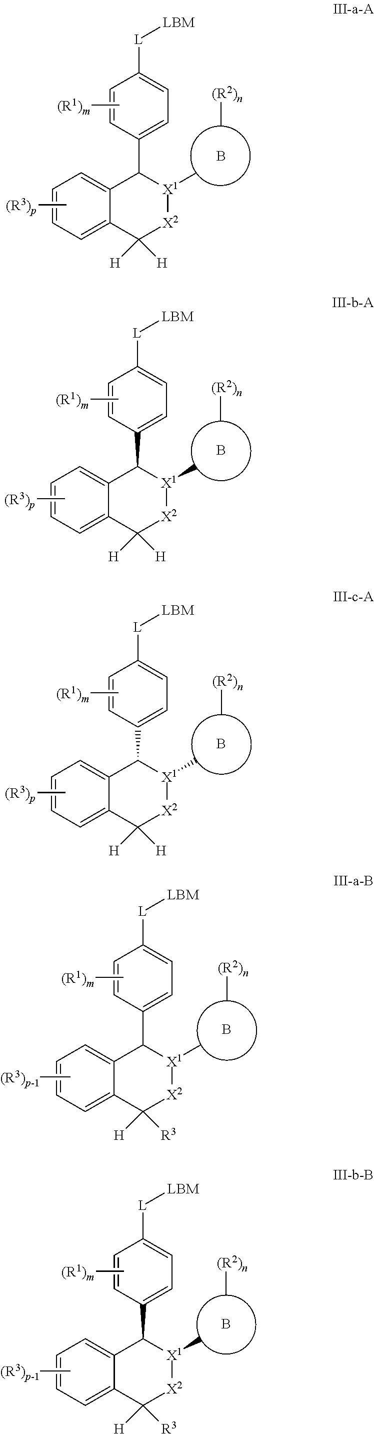

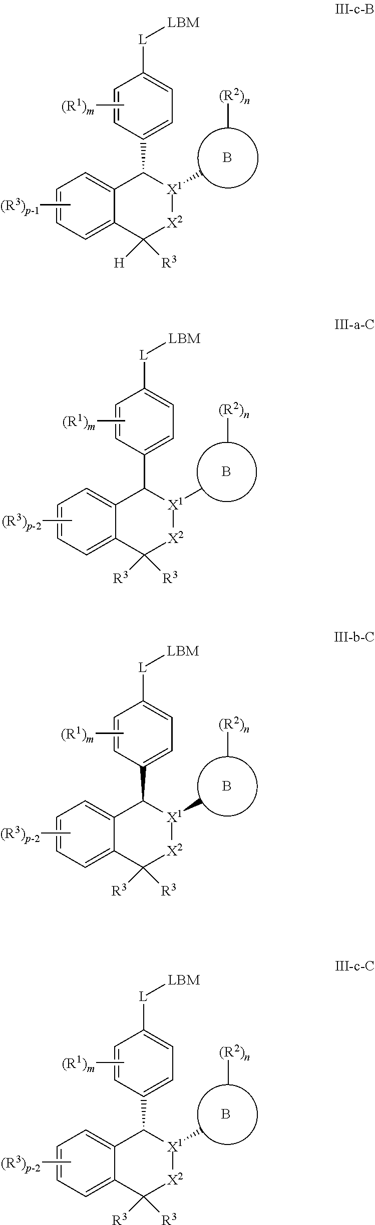

or a pharmaceutically acceptable salt thereof, provided that for formulae III-a-A, III-b-A and III-c-A when X¹ is CH or N and X² is CH₂, then Ring B is other than phenyl.

293. The compound of embodiment 292, wherein the compound is a compound of formula III-a-A, or a pharmaceutically acceptable salt thereof, provided that when X¹ is CH or N and X² is CH₂, then Ring B is other than phenyl.

294. The compound of embodiment 292, wherein the compound is a compound of formula III-b-A, or a pharmaceutically acceptable salt thereof, provided that when X¹ is CH or N and X² is CH₂, then Ring B is other than phenyl.

295. The compound of embodiment 292, wherein the compound is a compound of formula III-c-A, or a pharmaceutically acceptable salt thereof, provided that when X¹ is CH or N and X² is CH₂, then Ring B is other than phenyl.

296. The compound of embodiment 292, wherein the compound is a compound of formula III-a-B, or a pharmaceutically acceptable salt thereof.

297. The compound of embodiment 292, wherein the compound is a compound of formula III-b-B, or a pharmaceutically acceptable salt thereof.

298. The compound of embodiment 292, wherein the compound is a compound of formula III-c-B, or a pharmaceutically acceptable salt thereof.

299. The compound of embodiment 292, wherein the compound is a compound of formula III-a-C, or a pharmaceutically acceptable salt thereof.

300. The compound of embodiment 292, wherein the compound is a compound of formula III-b-C, or a pharmaceutically acceptable salt thereof.

301. The compound of embodiment 292, wherein the compound is a compound of formula III-c-C, or a pharmaceutically acceptable salt thereof.

302. The compound of any one of embodiments 1-287, wherein the compound is a compound of one of formulas III-d, III-e, III-f, III-g, III-h, III-i, III-j, III-k, or III-1:

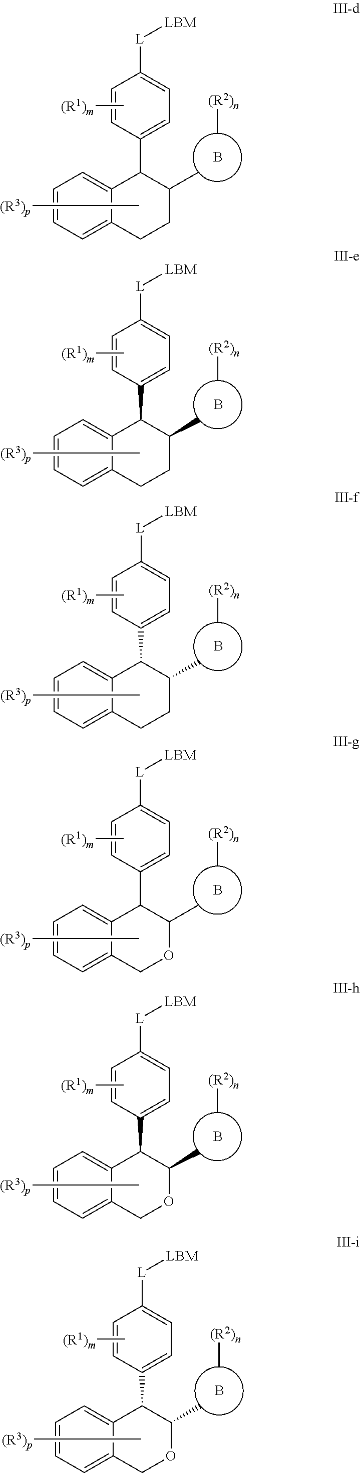

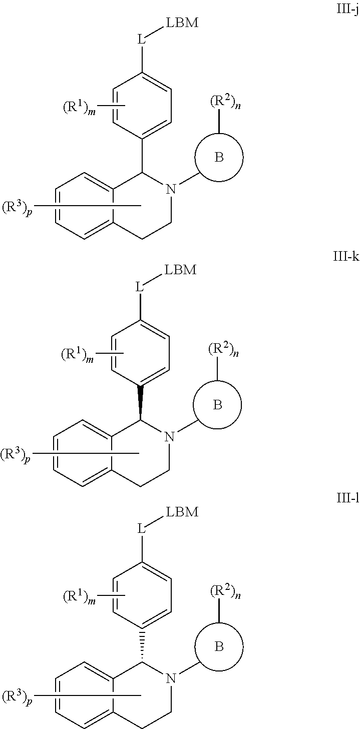

or a pharmaceutically acceptable salt thereof, provided that when the compound is of formula III-d, III-e, III-f, III-j, III-k or III-l, then Ring B is other than phenyl.

303. The compound of embodiment 302, wherein the compound is a compound of formula III-d, or a pharmaceutically acceptable salt thereof, provided that Ring B is other than phenyl.

304. The compound of embodiment 302, wherein the compound is a compound of formula III-e, or a pharmaceutically acceptable salt thereof, provided that Ring B is other than phenyl.

305. The compound of embodiment 302, wherein the compound is a compound of formula III-f, or a pharmaceutically acceptable salt thereof, provided that Ring B is other than phenyl.

306. The compound of embodiment 302, wherein the compound is a compound of formula III-g, or a pharmaceutically acceptable salt thereof.

307. The compound of embodiment 302, wherein the compound is a compound of formula III-h, or a pharmaceutically acceptable salt thereof.

308. The compound of embodiment 302, wherein the compound is a compound of formula III-i, or a pharmaceutically acceptable salt thereof.

309. The compound of embodiment 302, wherein the compound is a compound of formula III-j, or a pharmaceutically acceptable salt thereof, provided that Ring B is other than phenyl.

310. The compound of embodiment 302, wherein the compound is a compound of formula III-k, or a pharmaceutically acceptable salt thereof, provided that Ring B is other than phenyl.

311. The compound of embodiment 302, wherein the compound is a compound of formula III-l, or a pharmaceutically acceptable salt thereof, provided that Ring B is other than phenyl.

312. The compound of any one of embodiments 1-287, wherein the compound is a compound of formula III-d-A, III-e-A, III-f-A, III-d-B, III-e-B, III-f-B, III-d-C, III-e-C, III-f-C, III-g, III-h, III-i, III-j-A, III-k-A, or III-l-A:

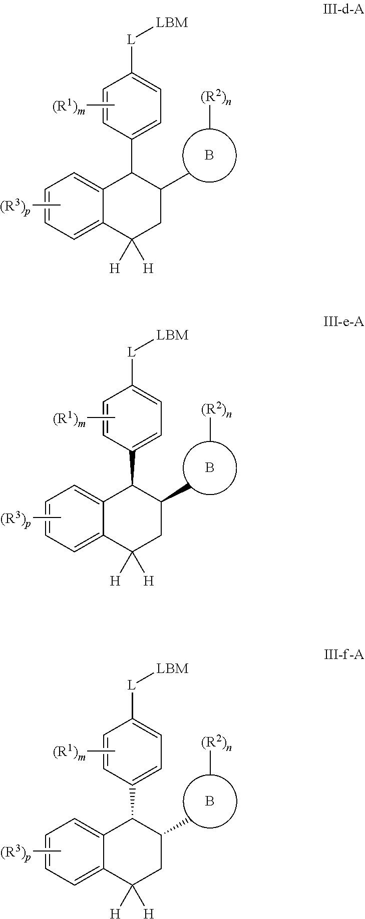

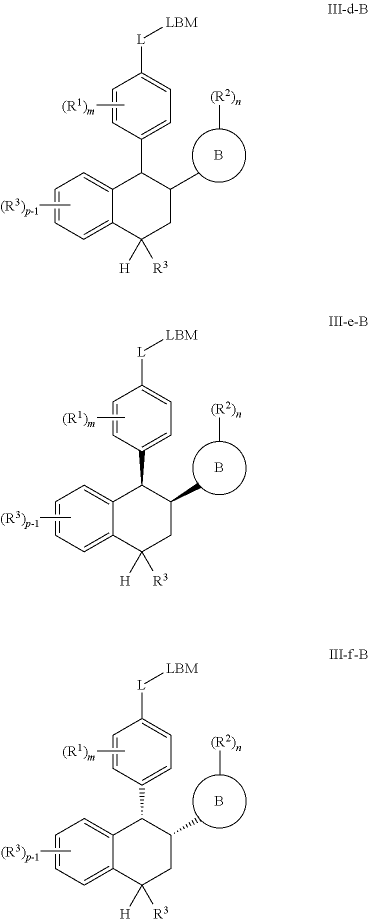

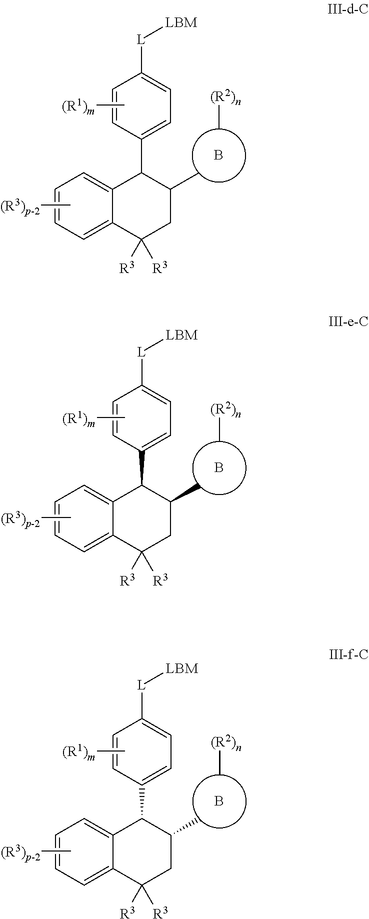

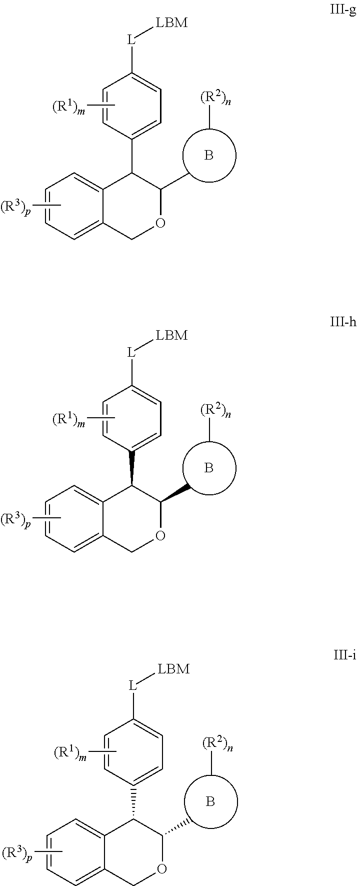

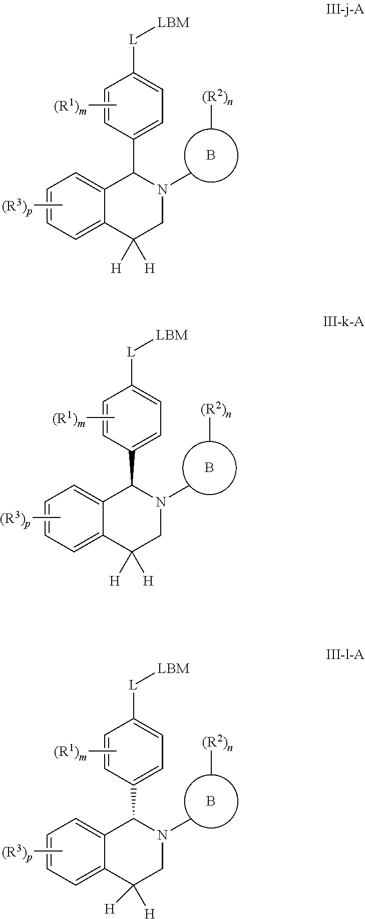

or a pharmaceutically acceptable salt thereof, provided that when the compound is of formula III-d-A, III-e-A, III-f-A, III-j-A, III-k-A, or III-l-A, then Ring B is other than phenyl.

313. The compound of embodiment 312, wherein the compound is a compound of formula III-d-A, or a pharmaceutically acceptable salt thereof, provided that Ring B is other than phenyl.

314. The compound of embodiment 312, wherein the compound is a compound of formula III-e-A, or a pharmaceutically acceptable salt thereof, provided that Ring B is other than phenyl.

315. The compound of embodiment 312, wherein the compound is a compound of formula III-f-A, or a pharmaceutically acceptable salt thereof, provided that Ring B is other than phenyl.

316. The compound of embodiment 312, wherein the compound is a compound of formula III-d-B, or a pharmaceutically acceptable salt thereof.

317. The compound of embodiment 312, wherein the compound is a compound of formula III-e-B, or a pharmaceutically acceptable salt thereof.

318. The compound of embodiment 312, wherein the compound is a compound of formula III-f-B, or a pharmaceutically acceptable salt thereof.

319. The compound of embodiment 312, wherein the compound is a compound of formula III-d-C, or a pharmaceutically acceptable salt thereof.

320. The compound of embodiment 312, wherein the compound is a compound of formula III-e-C, or a pharmaceutically acceptable salt thereof.

321. The compound of embodiment 312, wherein the compound is a compound of formula III-f-C, or a pharmaceutically acceptable salt thereof.

322. The compound of embodiment 302, wherein the compound is a compound of formula III-j-A, or a pharmaceutically acceptable salt thereof, provided that Ring B is other than phenyl.

323. The compound of embodiment 302, wherein the compound is a compound of formula III-k-A, or a pharmaceutically acceptable salt thereof, provided that Ring B is other than phenyl.

324. The compound of embodiment 302, wherein the compound is a compound of formula III-l-A, or a pharmaceutically acceptable salt thereof, provided that Ring B is other than phenyl 325. The compound of any one of embodiments 1-287, wherein the compound is a compound of one of formulas III-m, III-n, or III-o:

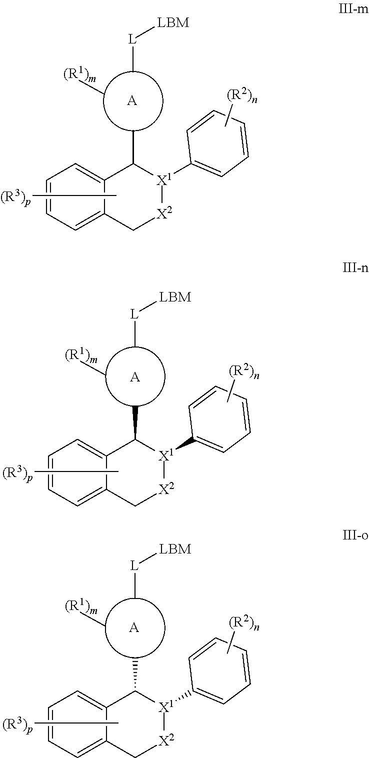

or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

326. The compound of embodiment 325, wherein the compound is a compound of formula III-m, or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

327. The compound of embodiment 325, wherein the compound is a compound of formula III-n, or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

328. The compound of embodiment 325, wherein the compound is a compound of formula III-o, or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

329. The compound of any one of embodiments 1-287, wherein is a compound of one of formulas III-m-A, III-n-A, or III-o-A, III-m-B, III-n-B, III-o-B, III-m-C, III-n-C, or III-o-C:

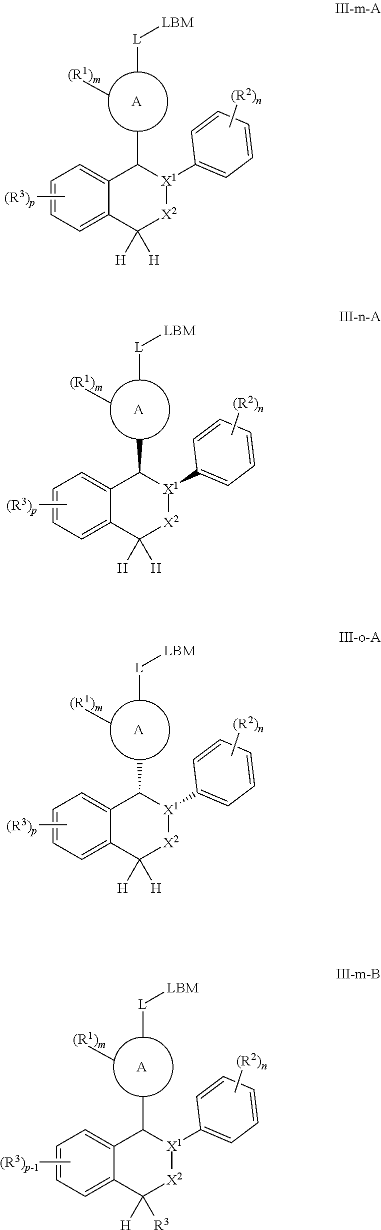

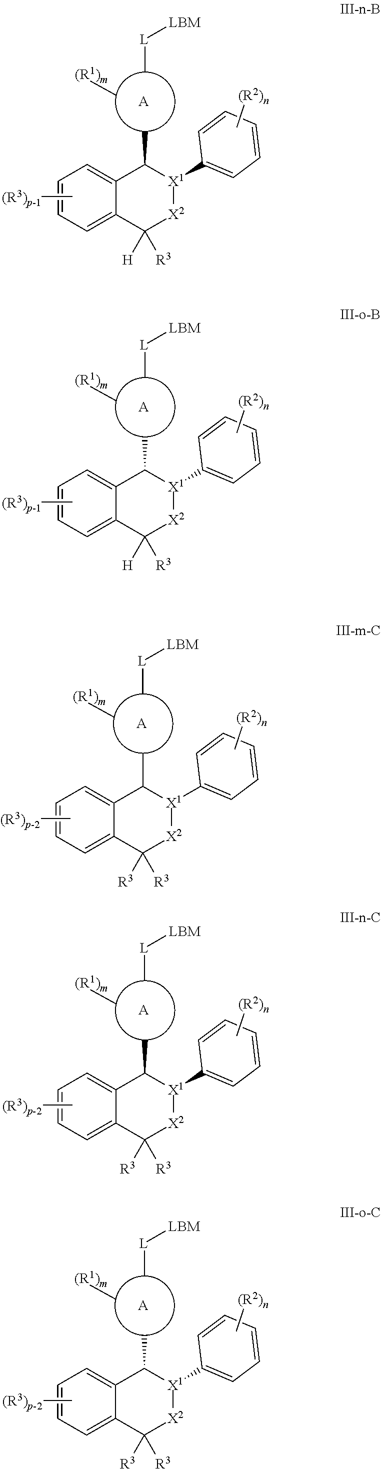

or a pharmaceutically acceptable salt thereof, provided that when the compound is of formulae III-m-A, III-n-A or III-o-A and X¹ is CH or N and X² is CH₂, then Ring A is other than phenyl or a 6 membered monocyclic heteroaryl ring having 1-3 nitrogen heteroatoms.

330. The compound of embodiment 329, wherein the compound is a compound of formula III-m-A, or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

331. The compound of embodiment 329, wherein the compound is a compound of formula III-n-A, or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

332. The compound of embodiment 329, wherein the compound is a compound of formula III-o-A, or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

333. The compound of embodiment 329, wherein the compound is a compound of formula III-m-B.

334. The compound of embodiment 329, wherein the compound is a compound of formula III-n-B.

335. The compound of embodiment 329, wherein the compound is a compound of formula III-o-B.

336. The compound of embodiment 329, wherein the compound is a compound of formula III-m-C.

337. The compound of embodiment 329, wherein the compound is a compound of formula III-n-C.

338. The compound of embodiment 329, wherein the compound is a compound of formula III-o-C.

339. The compound of any one of embodiments 1-287, wherein the compound is a compound of one of formulas III-p, III-q, III-r, III-s, III-t, III-u, III-v, III-w, or III-x:

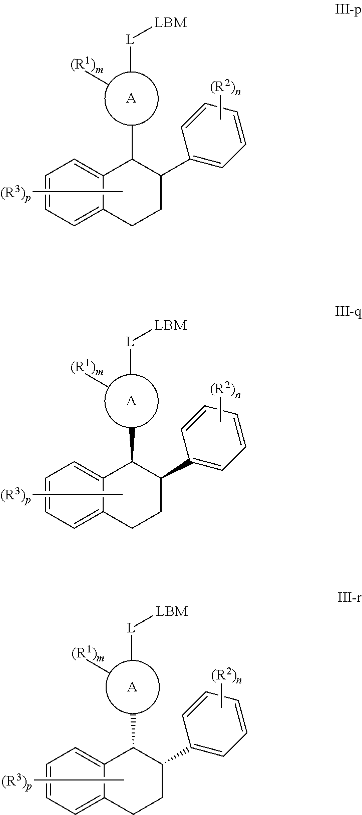

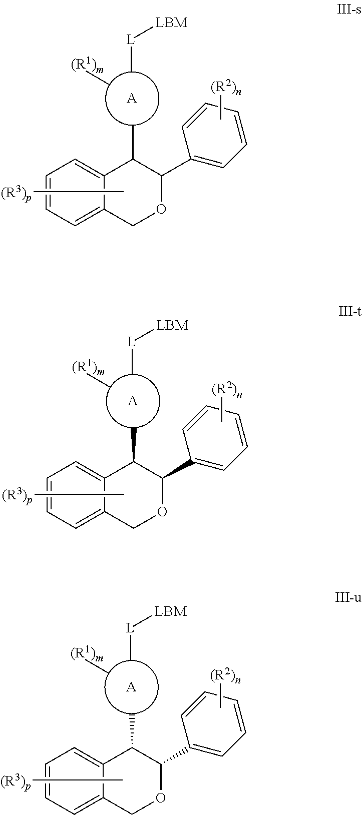

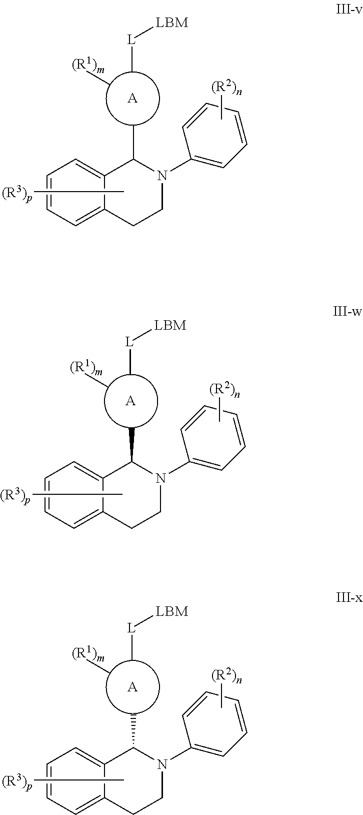

or a pharmaceutically acceptable salt thereof, provided that when the compound is of formulas III-p, III-q, III-r, III-v, III-w or III-x, then Ring A is other than phenyl or a 6 membered monocyclic heteroaryl ring having 1-3 nitrogen heteroatoms.

340. The compound of embodiment 339, wherein the compound is a compound of formula III-p, or a pharmaceutically acceptable salt thereof, provided that Ring A is other than phenyl or a 6 membered monocyclic heteroaryl ring having 1-3 nitrogen heteroatoms.

341. The compound of embodiment 339, wherein the compound is a compound of formula III-q, or a pharmaceutically acceptable salt thereof, provided that Ring A is other than phenyl or a 6 membered monocyclic heteroaryl ring having 1-3 nitrogen heteroatoms.

342. The compound of embodiment 339, wherein the compound is a compound of formula III-r, or a pharmaceutically acceptable salt thereof, provided that Ring A is other than phenyl or a 6 membered monocyclic heteroaryl ring having 1˜3 nitrogen heteroatoms.

343. The compound of embodiment 339, wherein the compound is a compound of formula III-s, or a pharmaceutically acceptable salt thereof.

344. The compound of embodiment 339, wherein the compound is a compound of formula III-t, or a pharmaceutically acceptable salt thereof.

345. The compound of embodiment 339, wherein the compound is a compound of formula III-u, or a pharmaceutically acceptable salt thereof.

346. The compound of embodiment 339, wherein the compound is a compound of formula III-v, or a pharmaceutically acceptable salt thereof, provided that Ring A is other than phenyl or a 6 membered monocyclic heteroaryl ring having 1-3 nitrogen heteroatoms.

347. The compound of embodiment 339, wherein the compound is a compound of formula III-w, or a pharmaceutically acceptable salt thereof, provided that Ring A is other than phenyl or a 6 membered monocyclic heteroaryl ring having 1-3 nitrogen heteroatoms.

348. The compound of embodiment 339, wherein the compound is a compound of formula III-x, or a pharmaceutically acceptable salt thereof, provided that Ring A is other than phenyl or a 6 membered monocyclic heteroaryl ring having 1-3 nitrogen heteroatoms.

349. The compound of any one of embodiments 1-287, wherein the compound is a compound of one of formulas III-p-A, III-q-A, III-r-A, III-p-B, III-q-B, III-r-B, III-p-C, III-q-C, III-r-C, III-s, III-t, III-u, III-v-A, III-w-A, or III-x-A:

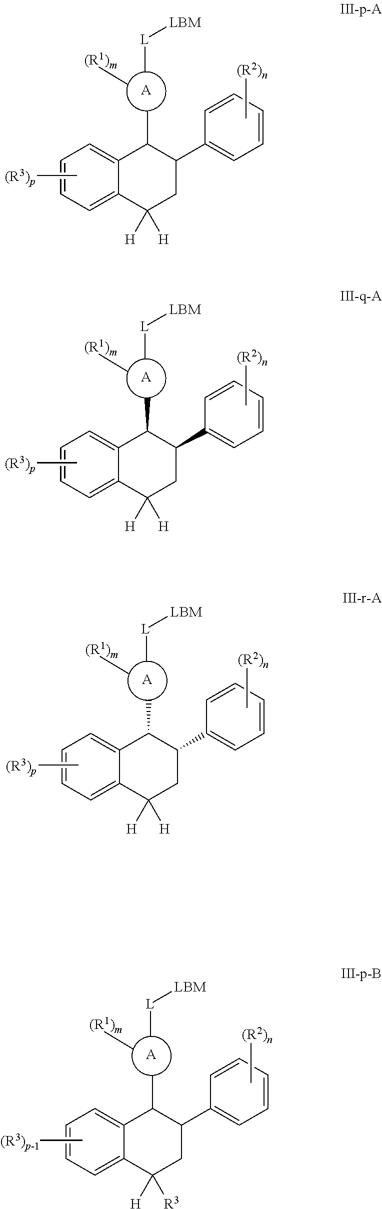

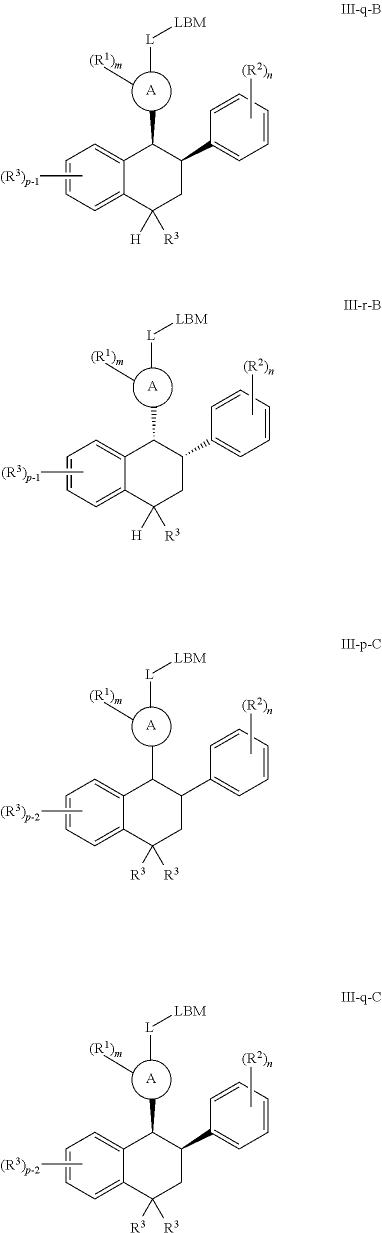

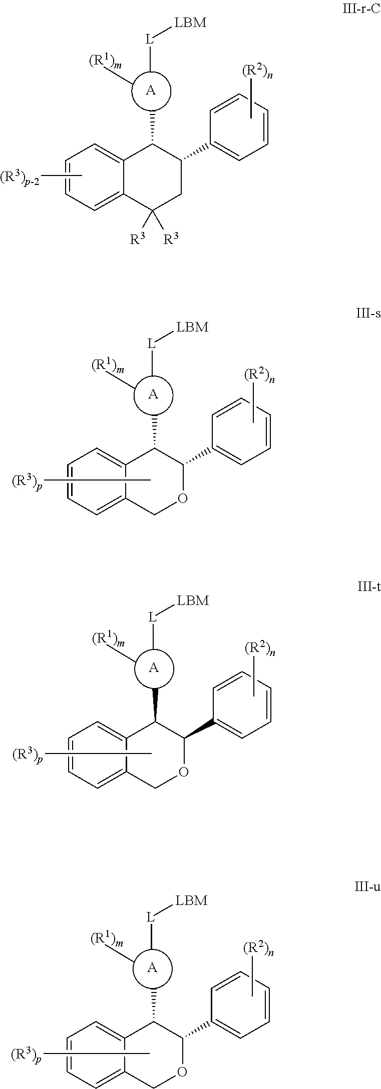

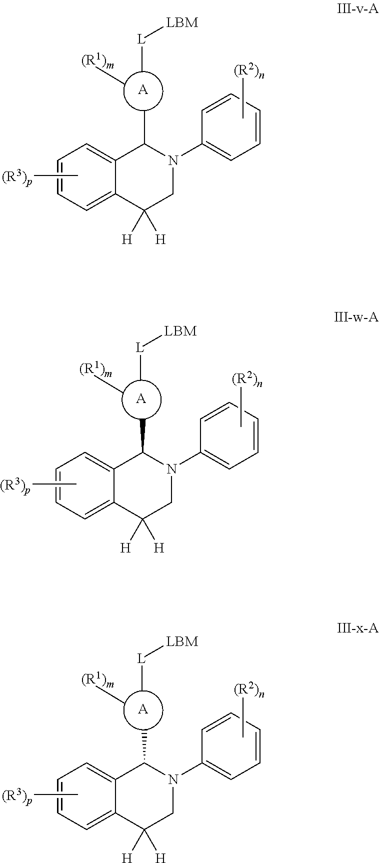

or a pharmaceutically acceptable salt thereof, provided that when the compound is of formulas III-p-A, III-q-A, III-r-A, III-v-A, III-w-A or III-x-A, then Ring A is other than phenyl or a 6 membered monocyclic heteroaryl ring having 1-3 nitrogen heteroatoms.

350. The compound of embodiment 349, wherein the compound is a compound of formula III-p-A, or a pharmaceutically acceptable salt thereof, provided that Ring A is other than phenyl or a 6 membered monocyclic heteroaryl ring having 1-3 nitrogen heteroatoms.

351. The compound of embodiment 349, wherein the compound is a compound of formula III-q-A, or a pharmaceutically acceptable salt thereof, provided that Ring A is other than phenyl or a 6 membered monocyclic heteroaryl ring having 1-3 nitrogen heteroatoms.

352. The compound of embodiment 349, wherein the compound is a compound of formula III-r-A, or a pharmaceutically acceptable salt thereof, provided that Ring A is other than phenyl or a 6 membered monocyclic heteroaryl ring having 1-3 nitrogen heteroatoms.

353. The compound of embodiment 349, wherein the compound is a compound of formula III-p-B, or a pharmaceutically acceptable salt thereof.

354. The compound of embodiment 349, wherein the compound is a compound of formula III-q-B, or a pharmaceutically acceptable salt thereof.

355. The compound of embodiment 349, wherein the compound is a compound of formula III-r-B, or a pharmaceutically acceptable salt thereof.

356. The compound of embodiment 349, wherein the compound is a compound of formula III-p-C, or a pharmaceutically acceptable salt thereof.

357. The compound of embodiment 349, wherein the compound is a compound of formula III-q-C, or a pharmaceutically acceptable salt thereof.

358. The compound of embodiment 349, wherein the compound is a compound of formula III-r-C, or a pharmaceutically acceptable salt thereof.

359. The compound of embodiment 349, wherein the compound is a compound of formula III-v-A, or a pharmaceutically acceptable salt thereof, provided that Ring A is other than phenyl or a 6 membered monocyclic heteroaryl ring having 1-3 nitrogen heteroatoms.

360. The compound of embodiment 349, wherein the compound is a compound of formula III-w-A, or a pharmaceutically acceptable salt thereof, provided that Ring A is other than phenyl or a 6 membered monocyclic heteroaryl ring having 1-3 nitrogen heteroatoms.

361. The compound of embodiment 349, wherein the compound is a compound of formula III-x-A, or a pharmaceutically acceptable salt thereof, provided that Ring A is other than phenyl or a 6 membered monocyclic heteroaryl ring having 1-3 nitrogen heteroatoms.

362. The compound of any one of embodiments 1-287, wherein the compound is a compound of one of formulas IV-a, IV-b, or IV-c:

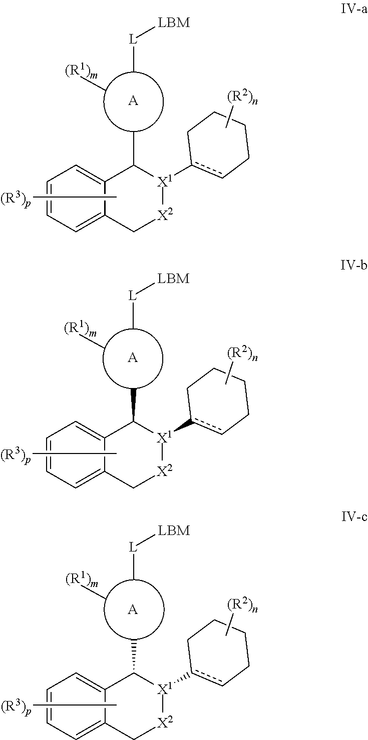

or a pharmaceutically acceptable salt thereof, wherein [symbol] represents a single or double bond.

363. The compound of embodiment 362, wherein the compound is a compound of formula IV-a, or a pharmaceutically acceptable salt thereof.

364. The compound of embodiment 362, wherein the compound is a compound of formula IV-b, or a pharmaceutically acceptable salt thereof.

365. The compound of embodiment 362, wherein the compound is a compound of formula IV-c, or a pharmaceutically acceptable salt thereof.

366. The compound of any one of embodiments 1-287, wherein the compound is a compound of one of formulas IV-d, IV-e, IV-f, IV-g, IV-h, IV-i, IV-j, IV-k, or IV-l:

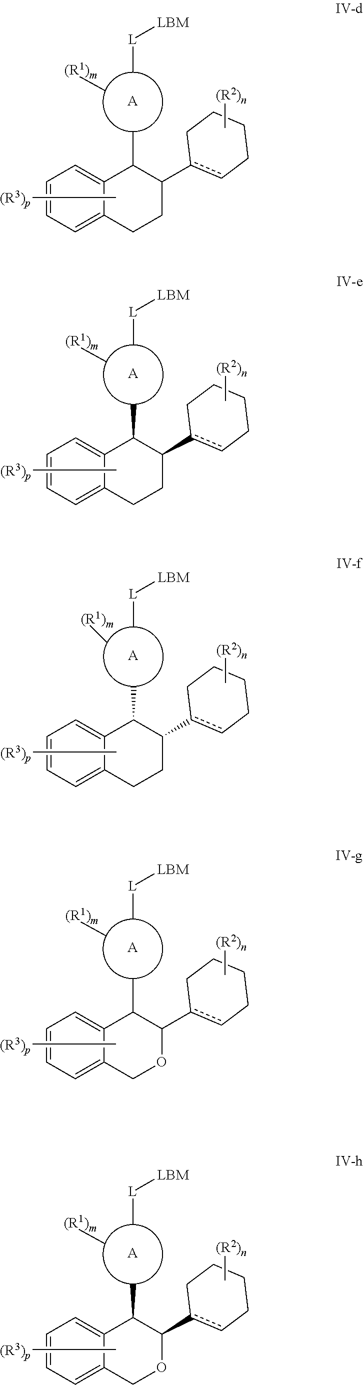

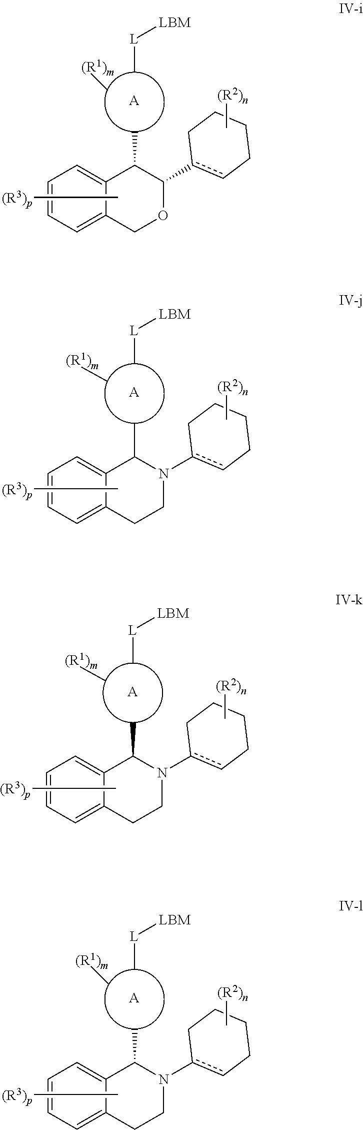

or a pharmaceutically acceptable salt thereof, wherein [symbol] represents a single or double bond.

367. The compound of embodiment 366, wherein the compound is a compound of formula IV-d, or a pharmaceutically acceptable salt thereof.

368. The compound of embodiment 366, wherein the compound is a compound of formula IV-e, or a pharmaceutically acceptable salt thereof.

369. The compound of embodiment 366, wherein the compound is a compound of formula IV-f, or a pharmaceutically acceptable salt thereof.

370. The compound of embodiment 366, wherein the compound is a compound of formula IV-g, or a pharmaceutically acceptable salt thereof.

371. The compound of embodiment 366, wherein the compound is a compound of formula IV-h, or a pharmaceutically acceptable salt thereof.

372. The compound of embodiment 366, wherein the compound is a compound of formula IV-i, or a pharmaceutically acceptable salt thereof.

373. The compound of embodiment 366, wherein the compound is a compound of formula IV-j, or a pharmaceutically acceptable salt thereof.

374. The compound of embodiment 366, wherein the compound is a compound of formula IV-k, or a pharmaceutically acceptable salt thereof.

375. The compound of embodiment 366, wherein the compound is a compound of formula IV-l, or a pharmaceutically acceptable salt thereof.

376. The compound of any one of embodiments 1-287, wherein the compound is a compound of one of formulas IV-m, IV-n, or IV-o:

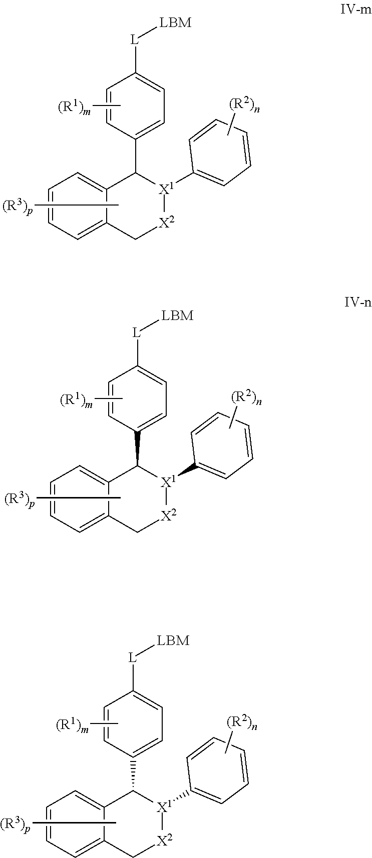

or a pharmaceutically acceptable salt thereof, provided that when X¹ is CH or N then X² is other than CH₂.

377. The compound of embodiment 376, wherein the compound is a compound of formula IV-m, or a pharmaceutically acceptable salt thereof, provided that when X¹ is CH or N then X² is other than CH₂.

378. The compound of embodiment 376, wherein the compound is a compound of formula IV-n, or a pharmaceutically acceptable salt thereof, provided that when X¹ is CH or N then X² is other than CH₂.

379. The compound of embodiment 376, wherein the compound is a compound of formula IV-o, or a pharmaceutically acceptable salt thereof, provided that when X¹ is CH or N then X² is other than CH₂.

380. The compound of any one of embodiments 1-287, wherein the compound is a compound of formula IV-m-A, IV-n-A, IV-o-A, IV-m-B, IV-n-B, IV-o-B, IV-m-C, IV-n-C, or IV-o-C:

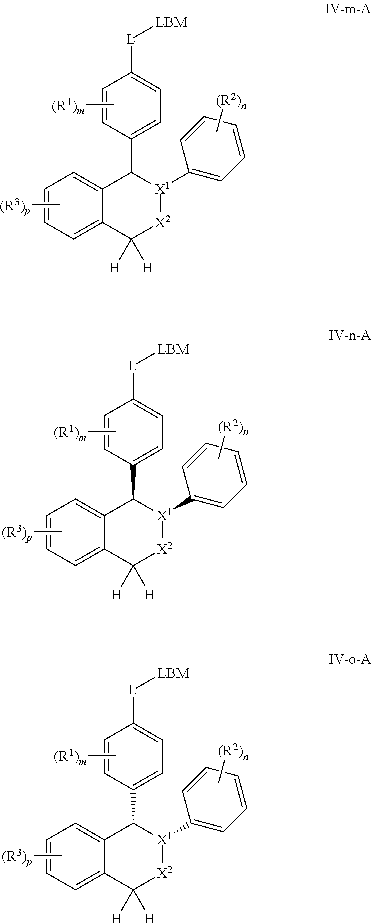

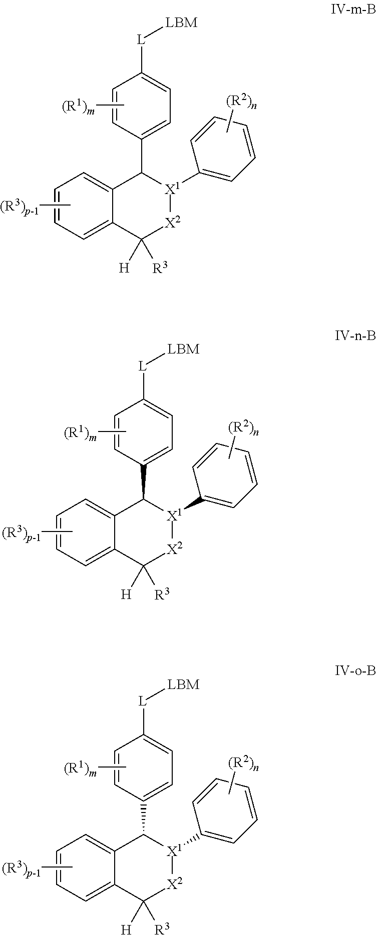

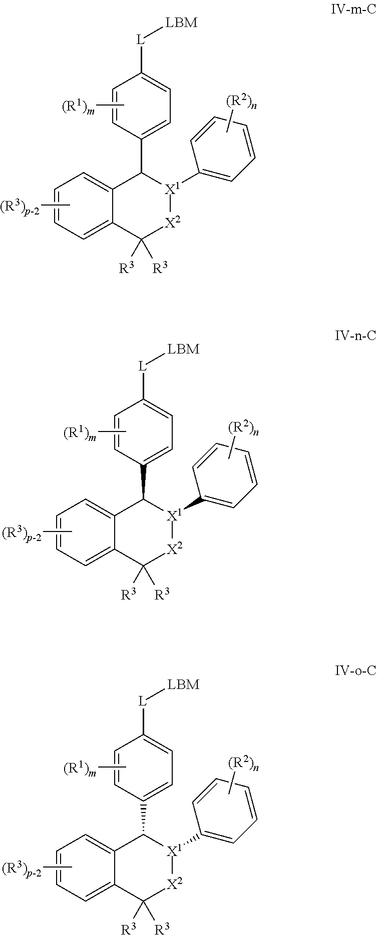

or a pharmaceutically acceptable salt thereof, provided that when the compound is of formula IV-m-A, IV-n-A or IV-o-A and X¹ is CH or N then X² is other than CH₂.

381. The compound of embodiment 380, wherein the compound is a compound of formula IV-m-A, or a pharmaceutically acceptable salt thereof, provided that when X¹ is CH or N then X² is other than CH₂.

382. The compound of embodiment 380, wherein the compound is a compound of formula IV-n-A, or a pharmaceutically acceptable salt thereof, provided that when X¹ is CH or N then X² is other than CH₂.

383. The compound of embodiment 380, wherein the compound is a compound of formula IV-o-A, or a pharmaceutically acceptable salt thereof, provided that when X¹ is CH or N then X² is other than CH₂.

384. The compound of embodiment 380, wherein the compound is a compound of formula IV-m-B, or a pharmaceutically acceptable salt thereof.

385. The compound of embodiment 380, wherein the compound is a compound of formula IV-n-B, or a pharmaceutically acceptable salt thereof.

386. The compound of embodiment 380, wherein the compound is a compound of formula IV-o-B, or a pharmaceutically acceptable salt thereof.

387. The compound of embodiment 380, wherein the compound is a compound of formula IV-m-C, or a pharmaceutically acceptable salt thereof.

388. The compound of embodiment 380, wherein the compound is a compound of formula IV-n-C, or a pharmaceutically acceptable salt thereof.

389. The compound of embodiment 380, wherein the compound is a compound of formula IV-o-C, or a pharmaceutically acceptable salt thereof.

390. The compound of any one of embodiments 1-287, wherein the compound is a compound of one of formulas IV-s, IV-t, IV-u, IV-v, IV-w, or IV-x:

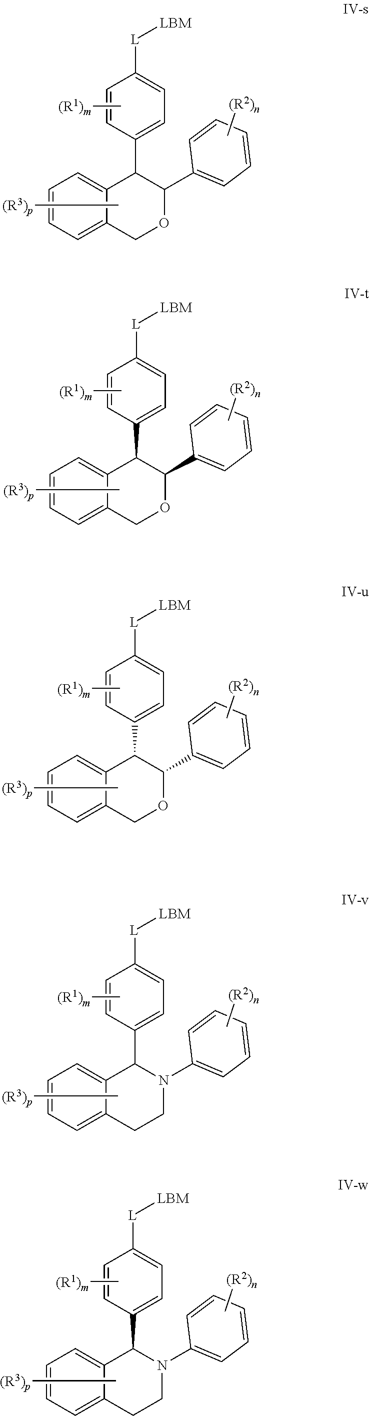

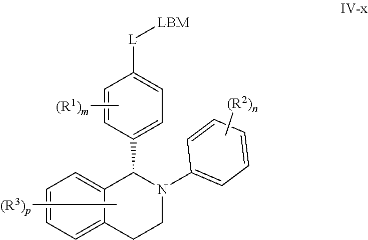

or a pharmaceutically acceptable salt thereof.

391. The compound of embodiment 390, wherein the compound is a compound of formula IV-s, or a pharmaceutically acceptable salt thereof.

392. The compound of embodiment 390, wherein the compound is a compound of formula IV-t, or a pharmaceutically acceptable salt thereof.

393. The compound of embodiment 390, wherein the compound is a compound of formula IV-u, or a pharmaceutically acceptable salt thereof.

394. The compound of embodiment 390, wherein the compound is a compound of formula IV-v, or a pharmaceutically acceptable salt thereof.

395. The compound of embodiment 390, wherein the compound is a compound of formula IV-w, or a pharmaceutically acceptable salt thereof.

396. The compound of embodiment 390, wherein the compound is a compound of formula IV-x, or a pharmaceutically acceptable salt thereof.

397. The compound of any one of embodiments 1-287, wherein the compound is a compound of formula IV-aa, IV-bb, IV-cc, IV-dd, IV-ee, IV-ff, IV-gg, IV-hh, IV-ii, IV-jj, IV-kk, or IV-ll:

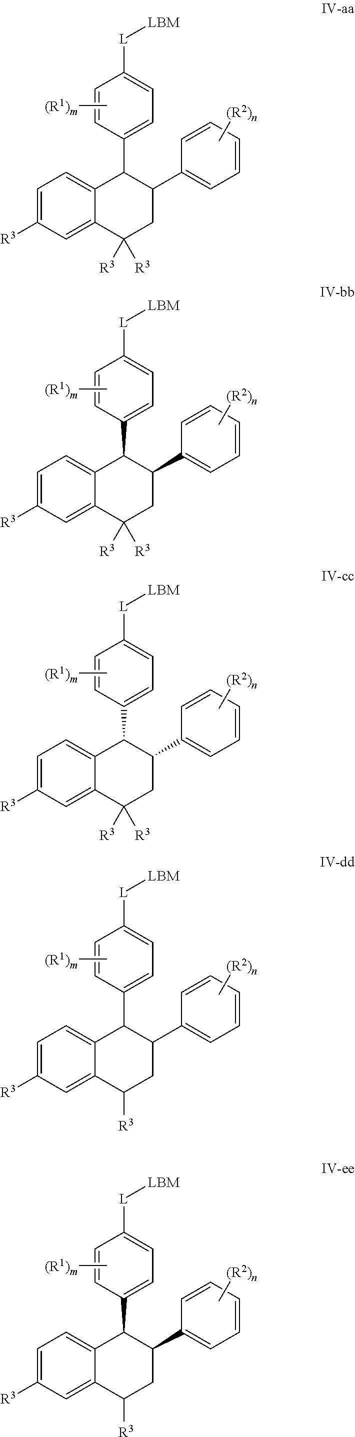

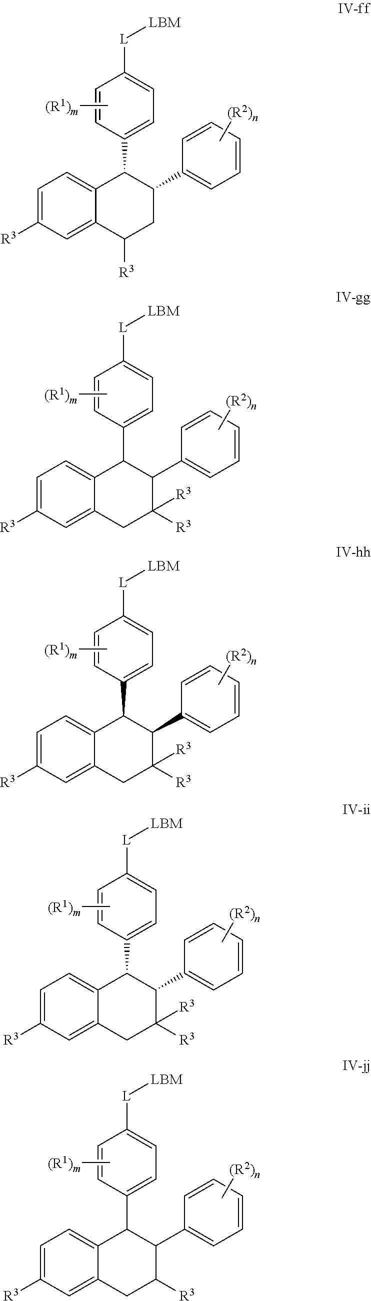

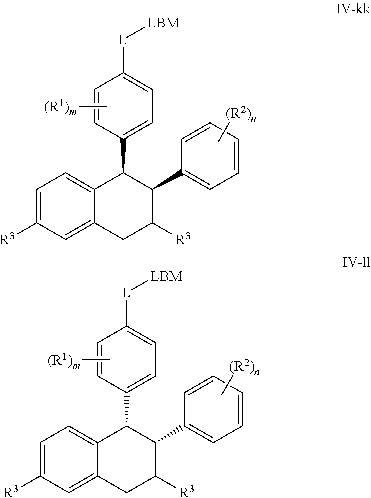

or a pharmaceutically acceptable salt thereof.

398. The compound of embodiment 397, wherein the compound is a compound of formula IV-aa, or a pharmaceutically acceptable salt thereof.

399. The compound of embodiment 397, wherein the compound is a compound of formula IV-bb, or a pharmaceutically acceptable salt thereof.

400. The compound of embodiment 397, wherein the compound is a compound of formula IV-cc, or a pharmaceutically acceptable salt thereof.

401. The compound of embodiment 397, wherein the compound is a compound of formula IV-dd, or a pharmaceutically acceptable salt thereof.

402. The compound of embodiment 397, wherein the compound is a compound of formula IV-ee, or a pharmaceutically acceptable salt thereof.

403. The compound of embodiment 397, wherein the compound is a compound of formula IV-ff, or a pharmaceutically acceptable salt thereof.

404. The compound of embodiment 397, wherein the compound is a compound of formula IV-gg, or a pharmaceutically acceptable salt thereof.

405. The compound of embodiment 397, wherein the compound is a compound of formula IV-hh, or a pharmaceutically acceptable salt thereof.

406. The compound of embodiment 397, wherein the compound is a compound of formula IV-ii, or a pharmaceutically acceptable salt thereof.

407. The compound of embodiment 397, wherein the compound is a compound of formula IV-jj, or a pharmaceutically acceptable salt thereof.

408. The compound of embodiment 397, wherein the compound is a compound of formula IV-kk, or a pharmaceutically acceptable salt thereof.

409. The compound of embodiment 397, wherein the compound is a compound of formula IV-ll, or a pharmaceutically acceptable salt thereof.

410. The compound of any one of embodiments 1-287, wherein the compound is a compound of one of formulas V-a, V-b, or V-c:

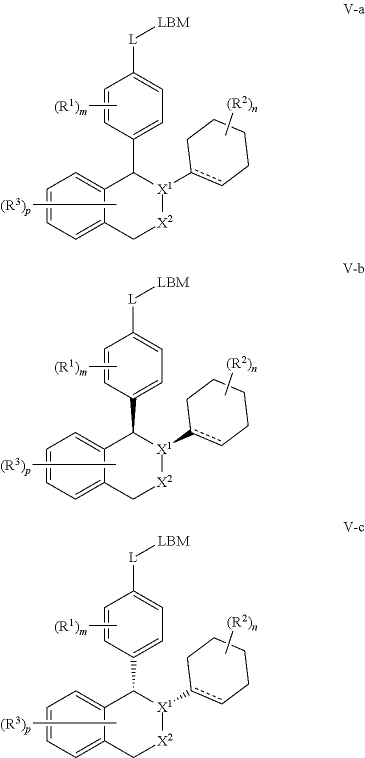

or a pharmaceutically acceptable salt thereof, wherein [symbol] represents a single or double bond.

411. The compound of embodiment 410, wherein the compound is a compound of formula V-a, or a pharmaceutically acceptable salt thereof.

412. The compound of embodiment 410, wherein the compound is a compound of formula V-b, or a pharmaceutically acceptable salt thereof.

413. The compound of embodiment 410, wherein the compound is a compound of formula V-c, or a pharmaceutically acceptable salt thereof.

414. The compound of any one of embodiments 1-287, wherein the compound is a compound of one of formulas V-d, V-e, V-f, V-g, V-h, V-i, V-j, V-k, or V-l:

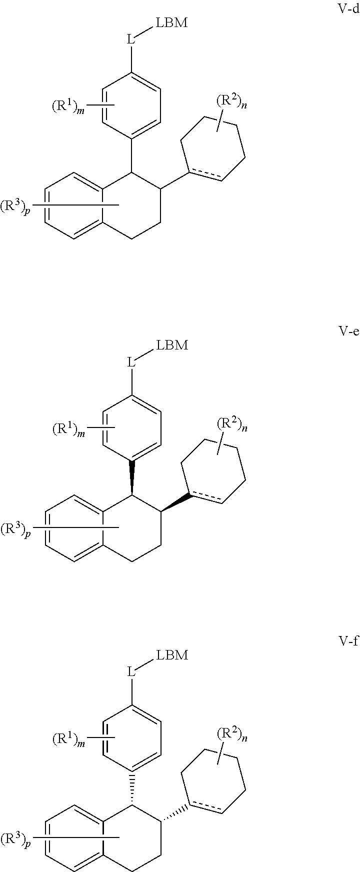

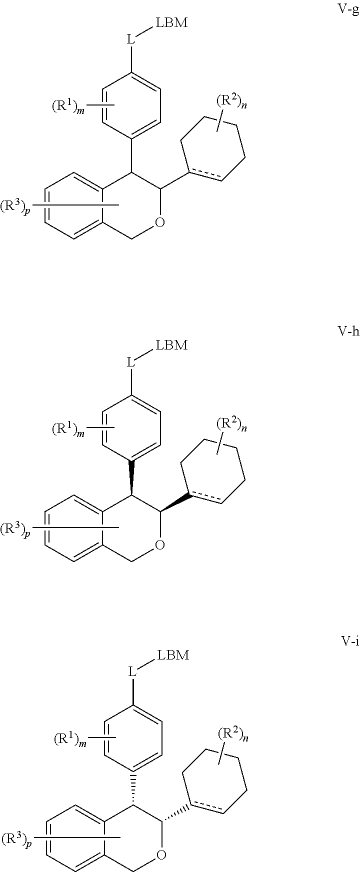

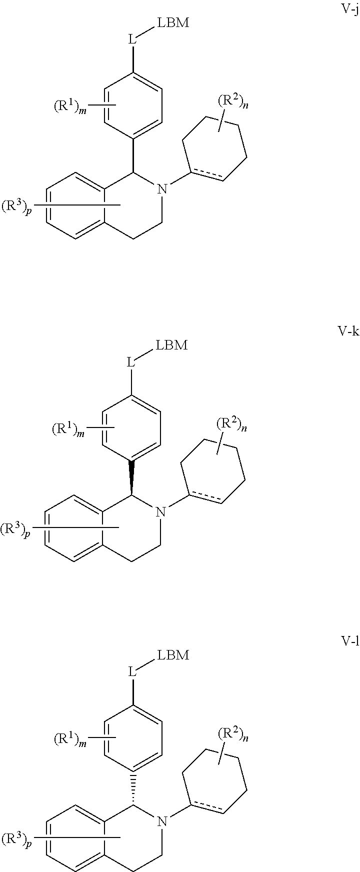

or a pharmaceutically acceptable salt thereof, wherein [symbol] represents a single or double bond.

415. The compound of embodiment 414, wherein the compound is a compound of formula V-d, or a pharmaceutically acceptable salt thereof.

416. The compound of embodiment 414, wherein the compound is a compound of formula V-e, or a pharmaceutically acceptable salt thereof.

417. The compound of embodiment 414, wherein the compound is a compound of formula V-f, or a pharmaceutically acceptable salt thereof.

418. The compound of embodiment 414, wherein the compound is a compound of formula V-g, or a pharmaceutically acceptable salt thereof.

419. The compound of embodiment 414, wherein the compound is a compound of formula V-h, or a pharmaceutically acceptable salt thereof.

420. The compound of embodiment 414, wherein the compound is a compound of formula V-i, or a pharmaceutically acceptable salt thereof.

421. The compound of embodiment 414, wherein the compound is a compound of formula V-j, or a pharmaceutically acceptable salt thereof.

422. The compound of embodiment 414, wherein the compound is a compound of formula V-k, or a pharmaceutically acceptable salt thereof.

423. The compound of embodiment 414, wherein the compound is a compound of formula V-l, or a pharmaceutically acceptable salt thereof.

424. The compound of any one of embodiments 1-287, wherein the compound is a compound of one of formulas VI-a, VI-b, or VI-e:

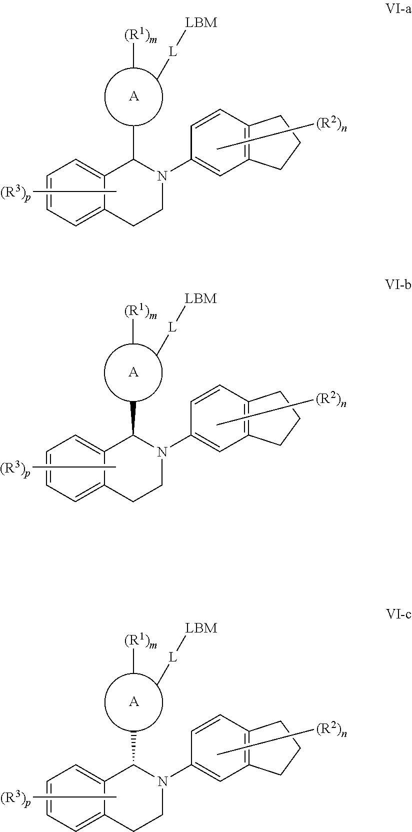

or a pharmaceutically acceptable salt thereof, wherein each of Ring A, R¹, R², R³, m, n, p, L, and LBM is as defined in embodiments and classes and subclasses herein.

425. The compound of embodiment 424, wherein the compound is a compound of formula VI-a, or a pharmaceutically acceptable salt thereof.

426. The compound of embodiment 424, wherein the compound is a compound of formula VI-b, or a pharmaceutically acceptable salt thereof.

427. The compound of embodiment 424, wherein the compound is a compound of formula VI-c, or a pharmaceutically acceptable salt thereof.

428. The compound of any one of embodiments 1-287, wherein the compound is a compound of one of formulas VI-d, VI-e, or VI-f:

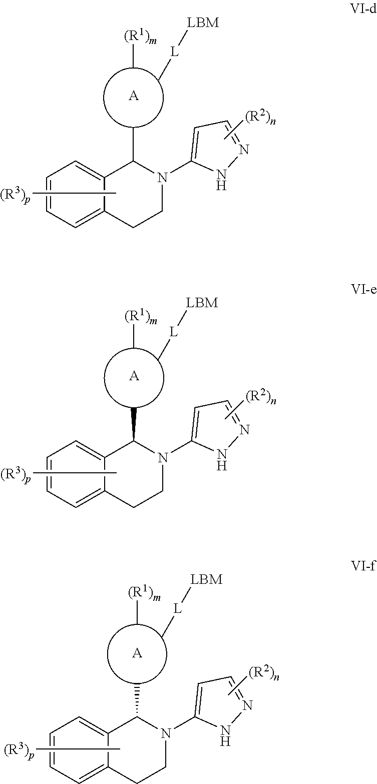

or a pharmaceutically acceptable salt thereof, wherein each of Ring A, R¹, R², R³, m, n, p, L, and LBM is as defined in embodiments and classes and subclasses herein.

429. The compound of embodiment 428, wherein the compound is a compound of formula VI-d, or a pharmaceutically acceptable salt thereof.

430. The compound of embodiment 428, wherein the compound is a compound of formula VI-e, or a pharmaceutically acceptable salt thereof.

431. The compound of embodiment 428, wherein the compound is a compound of formula VI-f, or a pharmaceutically acceptable salt thereof.

432. The compound of any one of embodiments 1-287, wherein the compound is a compound of one of formulas VI-g. VI-h, VI-i, VI-j, VI-k, or VI-l:

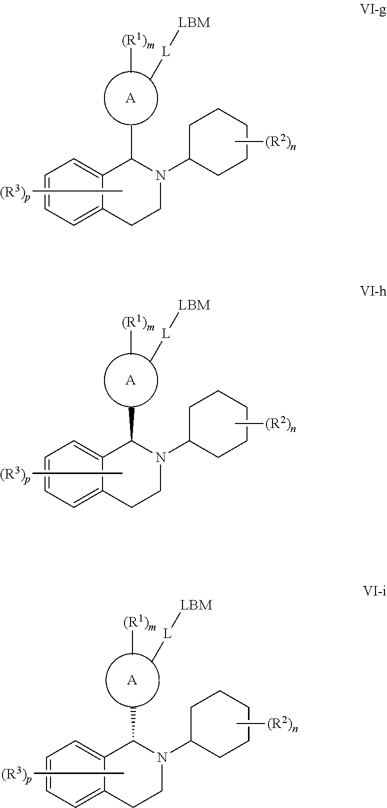

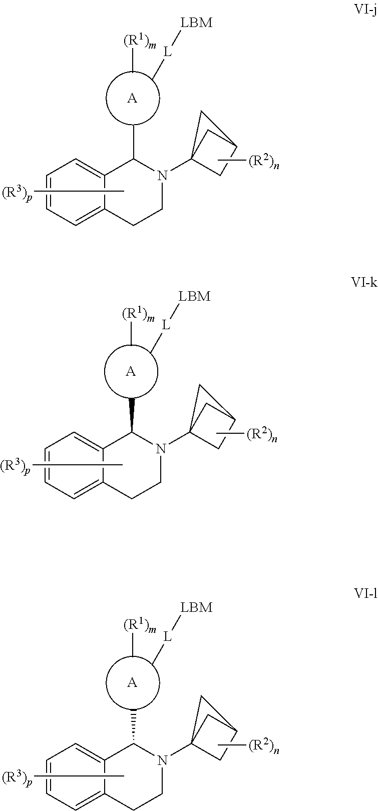

or a pharmaceutically acceptable salt thereof, wherein each of Ring A, R¹, R², R³, m, n, p, L, and LBM is as defined in embodiments and classes and subclasses herein.

433. The compound of embodiment 432, wherein the compound is a compound of formula VI-g, or a pharmaceutically acceptable salt thereof.

434. The compound of embodiment 432, wherein the compound is a compound of formula VI-h, or a pharmaceutically acceptable salt thereof.

435. The compound of embodiment 432, wherein the compound is a compound of formula VI-i, or a pharmaceutically acceptable salt thereof.

436. The compound of embodiment 432, wherein the compound is a compound of formula VI-j, or a pharmaceutically acceptable salt thereof.

437. The compound of embodiment 432, wherein the compound is a compound of formula VI-k, or a pharmaceutically acceptable salt thereof.

438. The compound of embodiment 432, wherein the compound is a compound of formula VI-l, or a pharmaceutically acceptable salt thereof.

439. The compound of any one of embodiments 1-287, wherein the compound is a compound of one of formulas VI-m, VI-n, or VI-o:

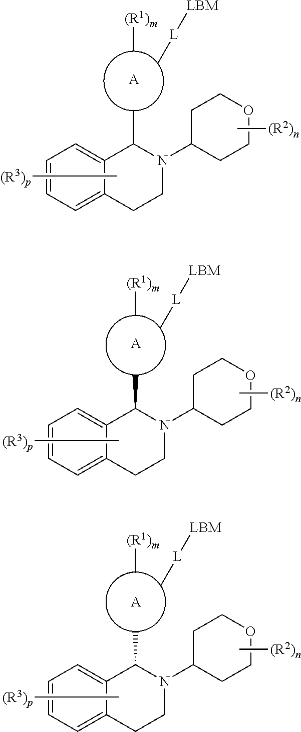

or a pharmaceutically acceptable salt thereof, wherein each of Ring A, R¹, R², R³, m, n, p, L, and LBM is as defined in embodiments and classes and subclasses herein.

440. The compound of embodiment 439, wherein the compound is a compound of formula VI-m, or a pharmaceutically acceptable salt thereof.

441. The compound of embodiment 439, wherein the compound is a compound of formula VI-n, or a pharmaceutically acceptable salt thereof.

442. The compound of embodiment 439, wherein the compound is a compound of formula VI-o, or a pharmaceutically acceptable salt thereof.

443. The compound of any one of embodiments 1-287, wherein the compound is a compound of one of formulas II-j-2, II-k-2, or II-l-2:

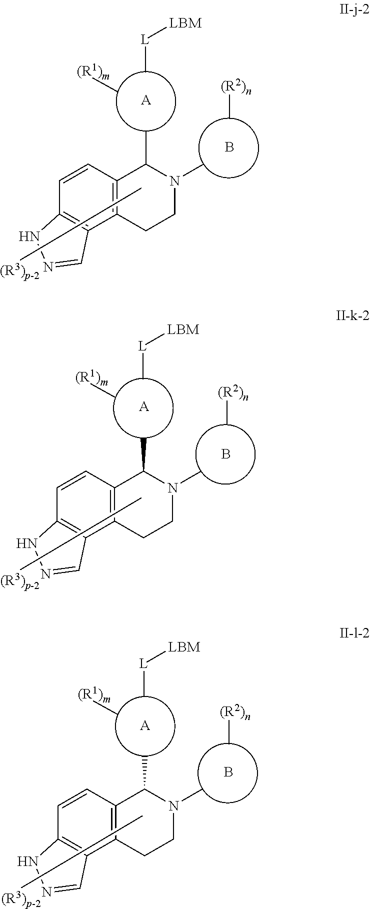

or a pharmaceutically acceptable salt thereof, wherein each of Ring A, Ring B, R¹, R², R³, m, n, p, L, and LBM is as defined in embodiments and classes and subclasses herein.

444. The compound of embodiment 443, wherein the compound is a compound of formula II-j-2, or a pharmaceutically acceptable salt thereof.

445. The compound of embodiment 443, wherein the compound is a compound of formula II-k-2, or a pharmaceutically acceptable salt thereof.

446. The compound of embodiment 443, wherein the compound is a compound of formula II-l-2, or a pharmaceutically acceptable salt thereof.

447. The compound of any one of embodiments 1-287, wherein the compound is a compound of one of formulas VII-a, VII-b, VII-c, VII-d, VII-e, or VII-f:

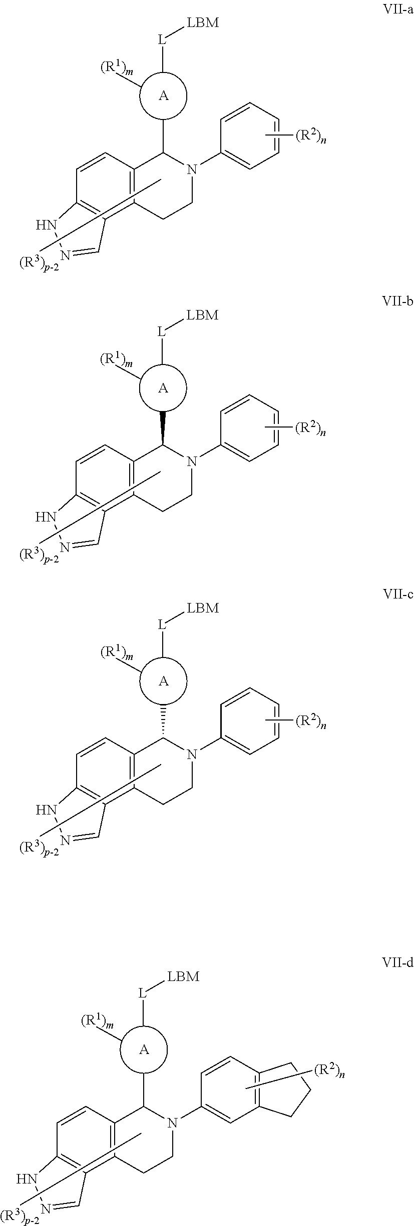

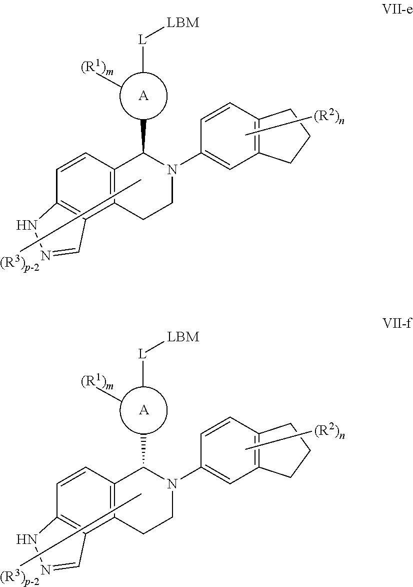

or a pharmaceutically acceptable salt thereof, wherein each of Ring A, R¹, R², R³, m, n, p, L, and LBM is as defined in embodiments and classes and subclasses herein.

448. The compound of embodiment 447, wherein the compound is a compound of formula VII-a, or a pharmaceutically acceptable salt thereof.

449. The compound of embodiment 447, wherein the compound is a compound of formula VII-b, or a pharmaceutically acceptable salt thereof.

150. The compound of embodiment 447, wherein the compound is a compound of formula VII-c, or a pharmaceutically acceptable salt thereof.

451. The compound of embodiment 447, wherein the compound is a compound of formula VII-d, or a pharmaceutically acceptable salt thereof.

452. The compound of embodiment 447, wherein the compound is a compound of formula VII-e, or a pharmaceutically acceptable salt thereof.

453. The compound of embodiment 447, wherein the compound is a compound of formula VII-f, or a pharmaceutically acceptable salt thereof.

454. The compound of any one of embodiments 1-287, wherein the compound is a compound of one of formulas VII-g, VII-h, or VII-i:

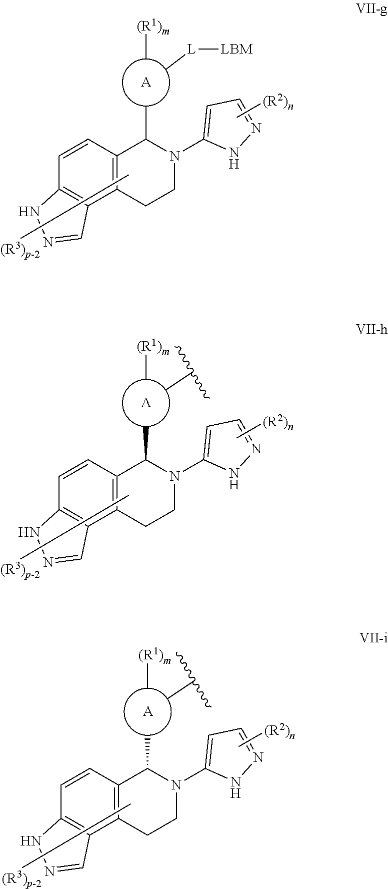

or a pharmaceutically acceptable salt thereof, wherein each of Ring A, R¹, R², R³, m, n, p, L, and LBM is as defined in embodiments and classes and subclasses herein.

455. The compound of embodiment 454, wherein the compound is a compound of formula VII-g, or a pharmaceutically acceptable salt thereof.

456. The compound of embodiment 454, wherein the compound is a compound of formula VII-h, or a pharmaceutically acceptable salt thereof.

457. The compound of embodiment 454, wherein the compound is a compound of formula VII-i, or a pharmaceutically acceptable salt thereof.

458. The compound of any one of embodiments 1-287, wherein the compound is a compound of one of formulas VII-j, VII-k, VII-l, VII-m, VII-n, or VII-o:

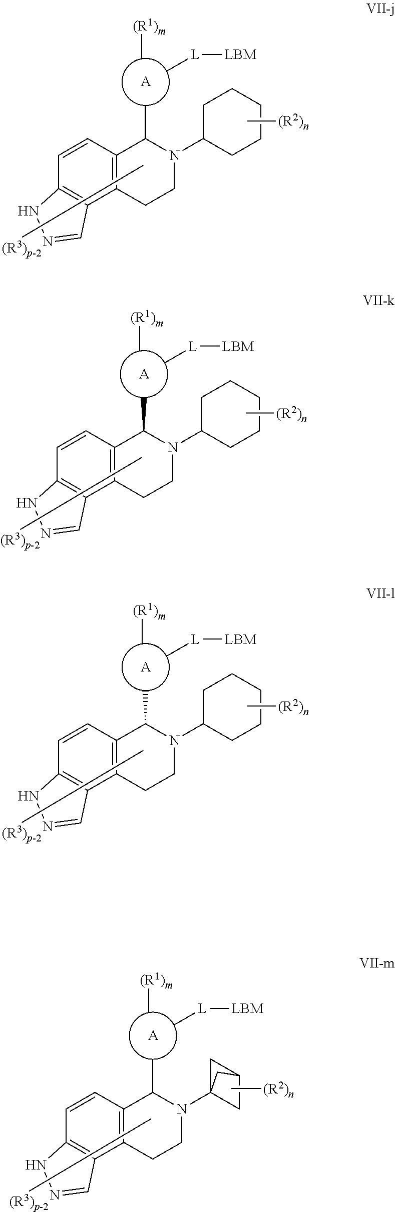

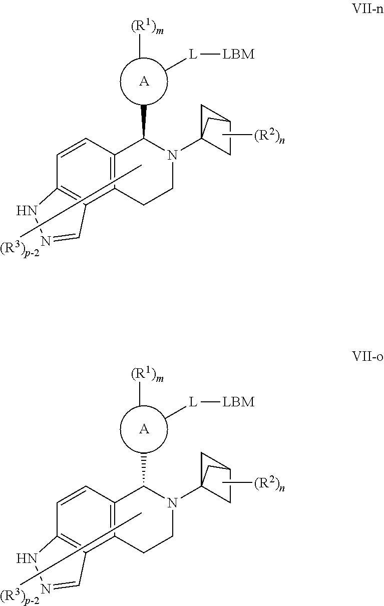

or a pharmaceutically acceptable salt thereof, wherein each of Ring A, R¹, R², R³, m, n, p, L, and LBM is as defined in embodiments and classes and subclasses herein.

459. The compound of embodiment 458, wherein the compound is a compound of formula VII-j, or a pharmaceutically acceptable salt thereof.

460. The compound of embodiment 458, wherein the compound is a compound of formula VII-k, or a pharmaceutically acceptable salt thereof.

461. The compound of embodiment 458, wherein the compound is a compound of formula VII-l, or a pharmaceutically acceptable salt thereof.

462. The compound of embodiment 458, wherein the compound is a compound of formula VII-m, or a pharmaceutically acceptable salt thereof.

463. The compound of embodiment 458, wherein the compound is a compound of formula VII-n, or a pharmaceutically acceptable salt thereof.

464. The compound of embodiment 458, wherein the compound is a compound of formula VII-o, or a pharmaceutically acceptable salt thereof.

465. The compound of any one of embodiments 1-287, wherein the compound is a compound of one of formulas VII-p, VII-q, or VII-r, respectively:

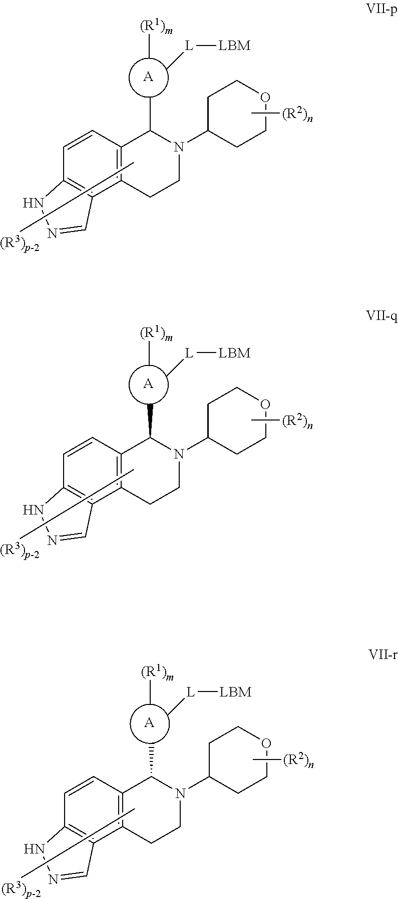

or a pharmaceutically acceptable salt thereof, wherein each of Ring A, R¹, R², R³, m, n, p, L, and LBM is as defined in embodiments and classes and subclasses herein.

466. The compound of embodiment 465, wherein the compound is a compound of formula VII-p, or a pharmaceutically acceptable salt thereof.

467. The compound of embodiment 465, wherein the compound is a compound of formula VII-q, or a pharmaceutically acceptable salt thereof.

468. The compound of embodiment 465, wherein the compound is a compound of formula VII-r, or a pharmaceutically acceptable salt thereof.

469. The compound of any one of embodiments 1-287, wherein the compound is a compound of one of formulas VIII-d, VIII-e, and VIII-f:

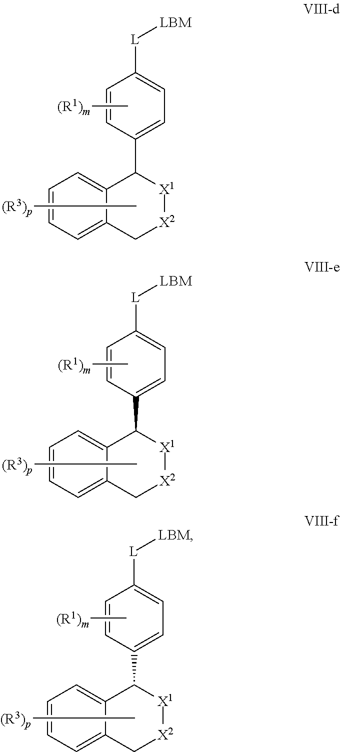

or a pharmaceutically acceptable salt thereof.

470. The compound of embodiment 469, wherein the compound is a compound of formula VIII-d, or a pharmaceutically acceptable salt thereof.

471. The compound of embodiment 469, wherein the compound is a compound of formula VIII-e, or a pharmaceutically acceptable salt thereof.

472. The compound of embodiment 469, wherein the compound is a compound of formula VIII-f, or a pharmaceutically acceptable salt thereof.

473. The compound of any one of embodiments 1-287, wherein the compound is a compound of one of formulas VIII-g, VIII-h, VIII-i, VIII-j, VIII-k or VIII-l:

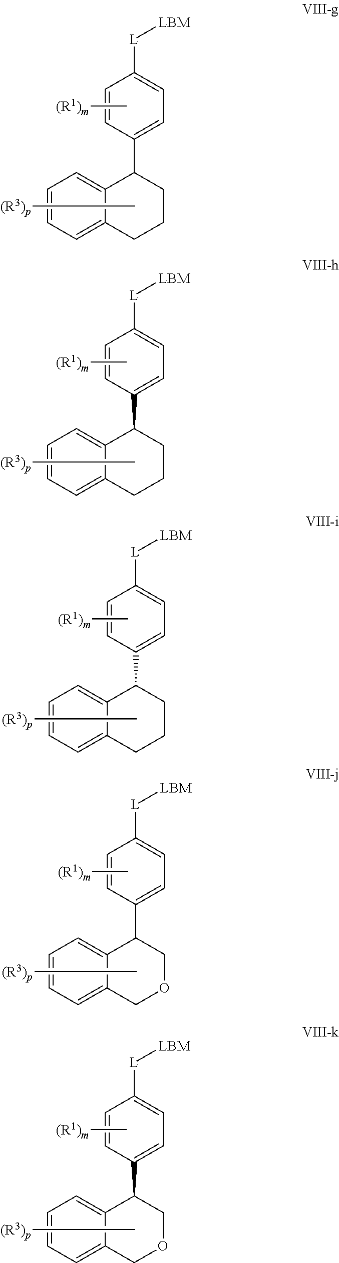

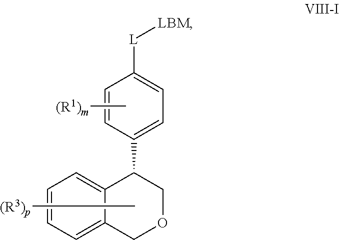

or a pharmaceutically acceptable salt thereof.

474. The compound of embodiment 473, wherein the compound is a compound of formula VIII-g, or a pharmaceutically acceptable salt thereof.

175. The compound of embodiment 473, wherein the compound is a compound of formula VIII-h, or a pharmaceutically acceptable salt thereof.

476. The compound of embodiment 473, wherein the compound is a compound of formula VIII-i, or a pharmaceutically acceptable salt thereof.

477. The compound of embodiment 473, wherein the compound is a compound of formula VIII-j, or a pharmaceutically acceptable salt thereof.

478. The compound of embodiment 473, wherein the compound is a compound of formula VIII-k, or a pharmaceutically acceptable salt thereof.

479. The compound of embodiment 473, wherein the compound is a compound of formula VIII-l, or a pharmaceutically acceptable salt thereof.

480. The compound of any one of embodiments 1-287, wherein the compound is a compound of one of formulas VIII-m, VIII-n, VIII-o, VIII-p, VIII-q, VIII-r, VIII-s, VIII-t, or VIII-u:

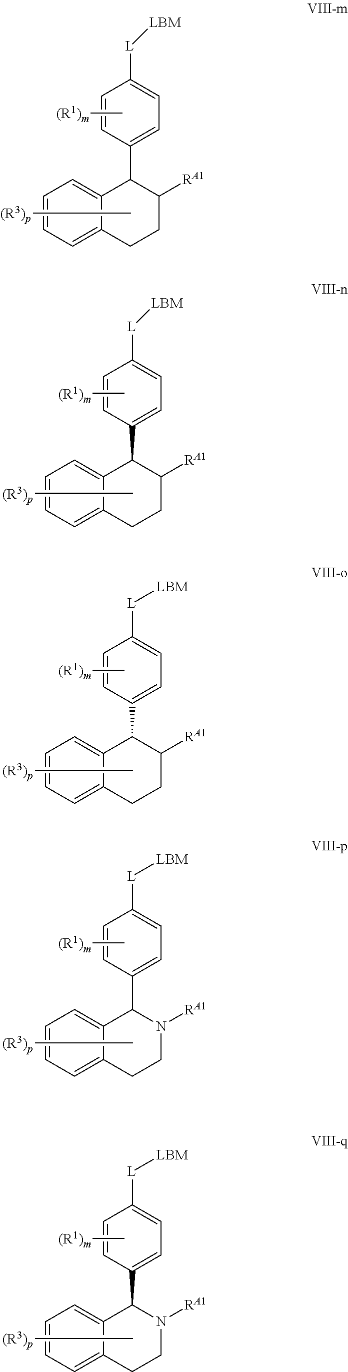

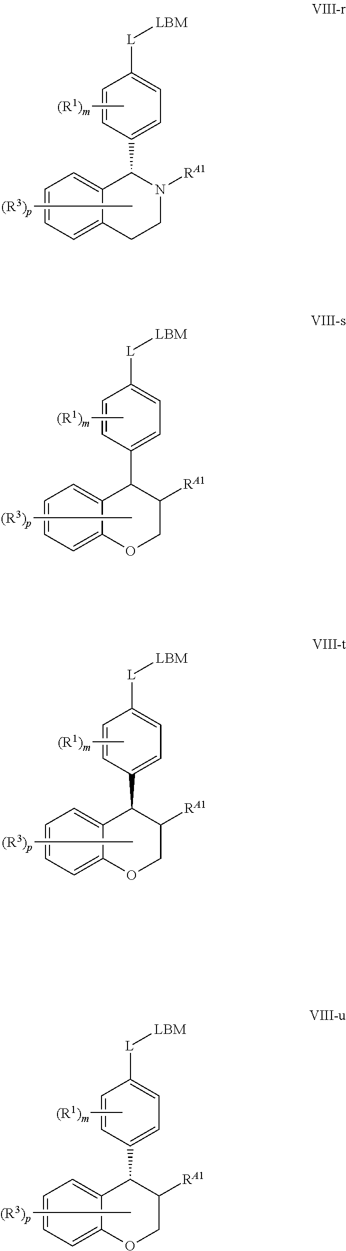

or a pharmaceutically acceptable salt thereof.

481. The compound of embodiment 480, wherein the compound is a compound of formula VIII-m, or a pharmaceutically acceptable salt thereof.

482. The compound of embodiment 480, wherein the compound is a compound of formula VIII-n, or a pharmaceutically acceptable salt thereof.

483. The compound of embodiment 480, wherein the compound is a compound of formula VIII-o, or a pharmaceutically acceptable salt thereof.

484. The compound of embodiment 480, wherein the compound is a compound of formula VIII-p, or a pharmaceutically acceptable salt thereof.

485. The compound of embodiment 480, wherein the compound is a compound of formula VIII-q, or a pharmaceutically acceptable salt thereof.

486. The compound of embodiment 480, wherein the compound is a compound of formula VIII-r, or a pharmaceutically acceptable salt thereof.

487. The compound of embodiment 480, wherein the compound is a compound of formula VIII-s, or a pharmaceutically acceptable salt thereof.

488. The compound of embodiment 480, wherein the compound is a compound of formula VIII-t, or a pharmaceutically acceptable salt thereof.

489. The compound of embodiment 480, wherein the compound is a compound of formula VIII-u, or a pharmaceutically acceptable salt thereof.

490. The compound of any one of embodiments 1-287, wherein the compound is a compound of one of formulas IX-d, IX-e, IX-f:

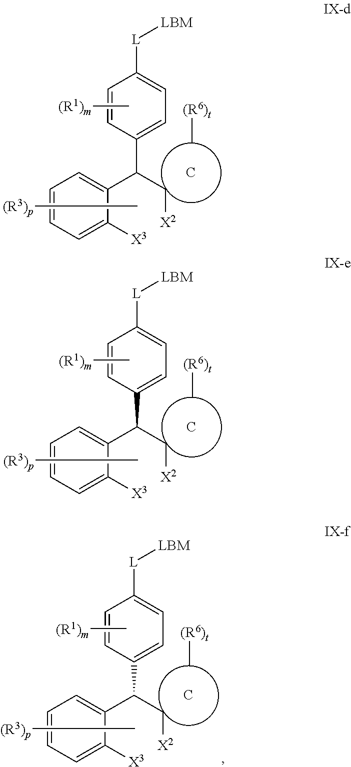

or a pharmaceutically acceptable salt thereof.

491. The compound of embodiment 490, wherein the compound is a compound of formula IX-d, or a pharmaceutically acceptable salt thereof.

492. The compound of embodiment 490, wherein the compound is a compound of formula IX-e, or a pharmaceutically acceptable salt thereof.

493. The compound of embodiment 490, wherein the compound is a compound of formula IX-f, or a pharmaceutically acceptable salt thereof.

494. The compound of any one of embodiments 1-287, wherein the compound is a compound of one of formulas IX-g, IX-h, IX-i, IX-j, IX-k, IX-l, IX-m, IX-n, IX-o:

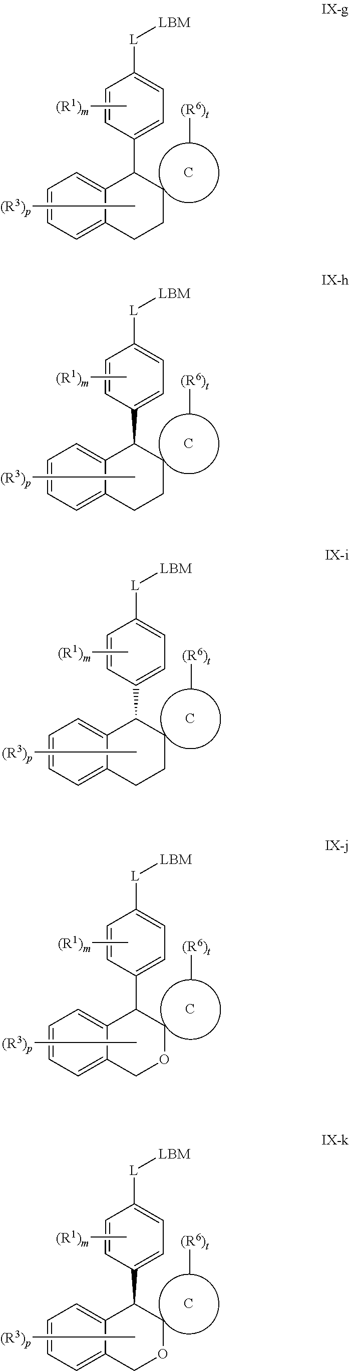

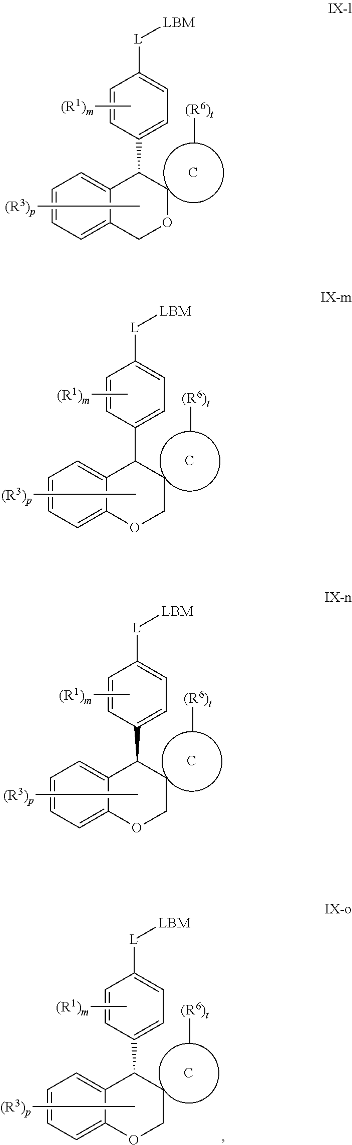

or a pharmaceutically acceptable salt thereof.

495. The compound of embodiment 494, wherein the compound is a compound of formula IX-g, or a pharmaceutically acceptable salt thereof.

496. The compound of embodiment 494, wherein the compound is a compound of formula IX-h, or a pharmaceutically acceptable salt thereof.

497. The compound of embodiment 494, wherein the compound is a compound of formula IX-i, or a pharmaceutically acceptable salt thereof.

498. The compound of embodiment 494, wherein the compound is a compound of formula IX-j, or a pharmaceutically acceptable salt thereof.

499. The compound of embodiment 494, wherein the compound is a compound of formula IX-k, or a pharmaceutically acceptable salt thereof.

500. The compound of embodiment 494, wherein the compound is a compound of formula IX-l, or a pharmaceutically acceptable salt thereof.

501. The compound of embodiment 494, wherein the compound is a compound of formula IX-m, or a pharmaceutically acceptable salt thereof.

502. The compound of embodiment 494, wherein the compound is a compound of formula IX-n, or a pharmaceutically acceptable salt thereof.

503. The compound of embodiment 494, wherein the compound is a compound of formula IX-o, or a pharmaceutically acceptable salt thereof.

504. The compound of any one of embodiments 1-287, wherein Ring C together with its R⁶ substituents is selected from:

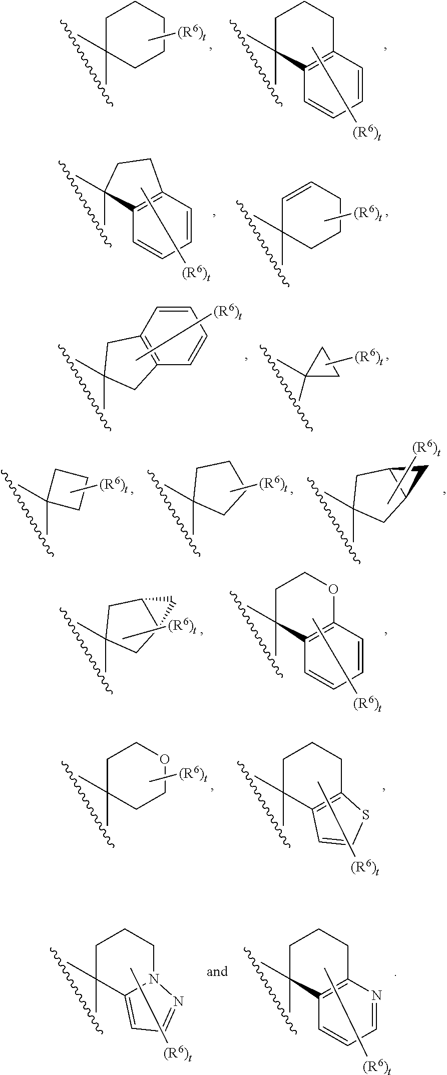

505. The compound of embodiment 504, wherein Ring C together with its R⁶ substituents is

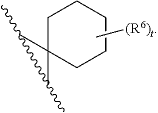

506. The compound of embodiment 504, wherein Ring C together with its R⁶ substituents is

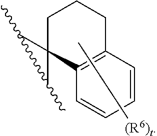

507. The compound of embodiment 504, wherein Ring C together with its R⁶ substituents is

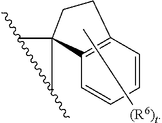

508. The compound of embodiment 504, wherein Ring C together with its R⁶ substituents is

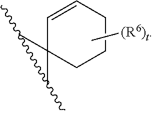

509. The compound of embodiment 504, wherein Ring C together with its R⁶ substituents is

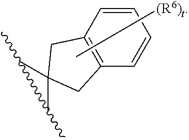

510. The compound of embodiment 504, wherein Ring C together with its R⁶ substituents is

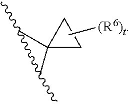

511. The compound of embodiment 504, wherein Ring C together with its R⁶ substituents is

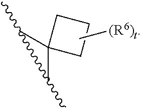

512. The compound of embodiment 504, wherein Ring C together with its R⁶ substituents is

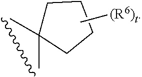

513. The compound of embodiment 504, wherein Ring C together with its R⁶ substituents is,

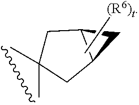

514. The compound of embodiment 504, wherein Ring C together with its R⁶ substituents is

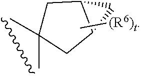

515. The compound of embodiment 504, wherein Ring C together with its R⁶ substituents is

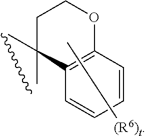

516. The compound of embodiment 504, wherein Ring C together with its R⁶ substituents is

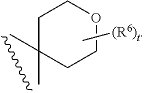

517. The compound of embodiment 504, wherein Ring C together with its R⁶ substituents is

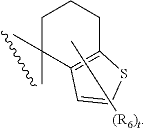

518. The compound of embodiment 504, wherein Ring C together with its R⁶ substituents is

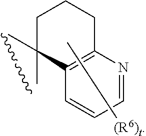

519. The compound of embodiment 504, wherein Ring C together with its R⁶ substituents is

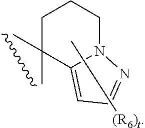

520. The compound of any one of embodiments 1-519, wherein X¹ is selected from CH and C(R^{A1}).

521. The compound of any one of embodiments 1-519, wherein X² is selected from O, CH₂, CH(R^{A3}), and C(R^{A3})₂, provided that X¹ and X² are not both simultaneously heteroatoms.

522. The compound of any one of embodiments 1-521, wherein Lis L is a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

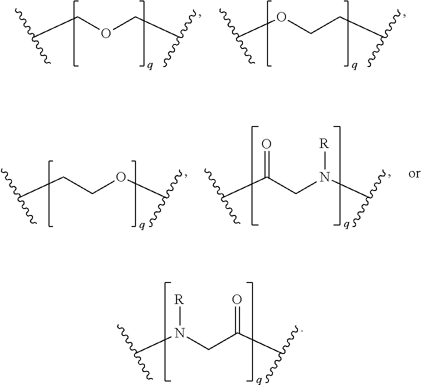

523. The compound of any one of embodiments 1-521, wherein L is a bivalent, saturated or unsaturated, straight or branched C₃₋₅ hydrocarbon chain, wherein 0, 1, 2 or 3 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

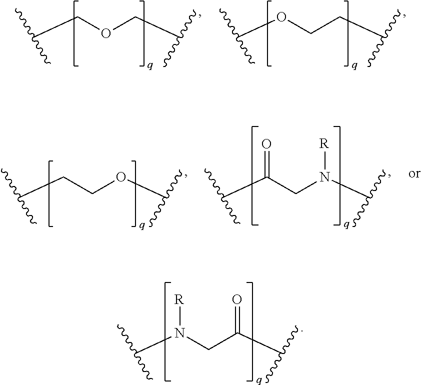

524. The compound of any one of embodiments 1-521, wherein L is a bivalent, saturated or unsaturated, straight or branched C₃₋₅ hydrocarbon chain, wherein 1, 2 or 3 methylene units of L are independently replaced by -Cy-, —CH(R)—, —C(R)₂—, —O— or —NR—.

525. The compound of any one of embodiments 1-521, wherein L is a bivalent, saturated or unsaturated, straight or branched C₃₋₅ hydrocarbon chain, wherein 1, 2 or 3 methylene units of L are independently replaced by -Cy- or —NR—.

526. The compound of any one of embodiments 1-521, wherein each -Cy- is independently an optionally substituted bivalent ring selected from a 4-7 membered saturated or partially unsaturated monocyclic carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated monocyclic heterocyclylenyl having 1-2 heteroatoms independently selected from nitrogen, oxygen, and sulfur, a 5-11 membered monocyclic saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur and a 6-10 membered bridged bicyclic saturated or partially unsaturated heterocyclylenyl having 1-2 heteroatoms independently selected from nitrogen, oxygen, and sulfur.

527. The compound of any one of embodiments 1-521, wherein each -Cy- is independently an optionally substituted bivalent ring selected from a 4-7 membered saturated or partially unsaturated monocyclic carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated monocyclic heterocyclylenyl containing 1-2 nitrogen atoms, a 5-11 membered monocyclic saturated or partially unsaturated spiro heterocyclylenyl containing 1-2 nitrogen atoms, an 8-10 membered bicyclic saturated or partially unsaturated heterocyclylenyl containing 1-2 nitrogen atoms, and a 6-10 membered bridged bicyclic saturated or partially unsaturated heterocyclylenyl containing 1-2 nitrogen atoms.

528. The compound of any one of embodiments 1-527, wherein the bivalent ring of each -Cy- is independently substituted with 0, 1 or 2 substituents independently selected from deuterium, C₁-C₄ alkyl, C₁-C₄ haloalkyl, C₃-C₇ cycloalkyl, —O—C₁-C₄ alkyl, halo, cyano, —OH, —NH₂, —N(H)(C₁-C₄ alkyl) and —N(C₁-C₄ alkyl)₂.

529. The compound of any one of embodiments 1-527, wherein the bivalent ring of each -Cy- is independently substituted with 0, 1 or 2 substituents independently selected from —C1-C4 alkyl, —C1-C4 haloalkyl, —C3-C7 cycloalkyl, —O—C1-C4 alkyl, halo, cyano, —OH, —NH2, —N(H)(C1-C4 alkyl) and —N(C1-C4 alkyl)2.

530. The compound of any one of embodiments 1-527, wherein the bivalent ring of each -Cy- is independently substituted with 0, 1 or 2 substituents independently selected from -Me, -Et, Pr, iPr, cyclopropyl, —CF₃, —OMe, —F, —Cl, —CN, —NH2, —NHMe and —NMe2.

531. The compound of any one of embodiments 1-527, wherein the bivalent ring of each -Cy- is independently substituted with 0, 1 or 2 substituents independently selected from -Me, —OMe and —F.

532. The compound of any one of embodiments 1-527, wherein the bivalent ring of each -Cy- is independently substituted with 0, 1 or 2 instances of -Me.

533. The compound of any one of embodiments 1-527, wherein the bivalent ring of each -Cy- is unsubstituted.

534. The compound of any one of embodiments 1-527, wherein the bivalent ring of each -Cy- is substituted with 1 or 2 substituents.

535. The compound of any one of embodiments 1-527, wherein L is

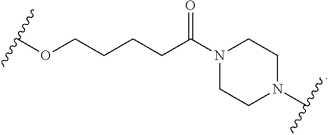 [SMILES: *OCCCCC(=O)N1CCN(*)CC1]

536. The compound of any one of embodiments 1-527, wherein L is selected from;

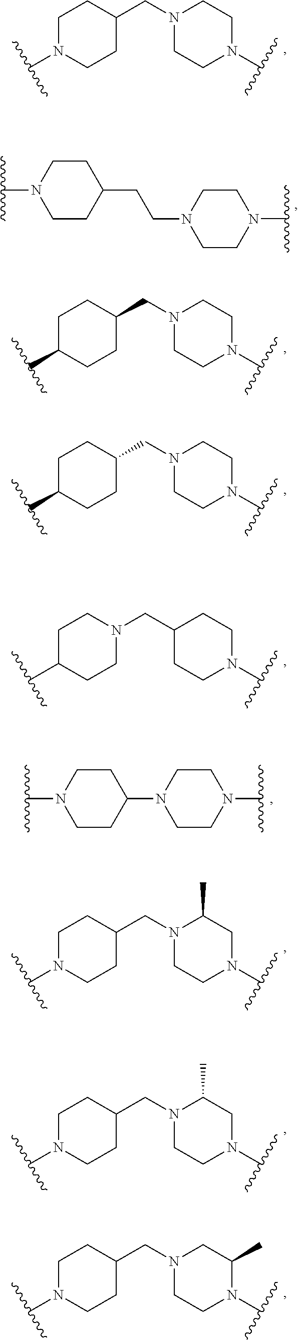

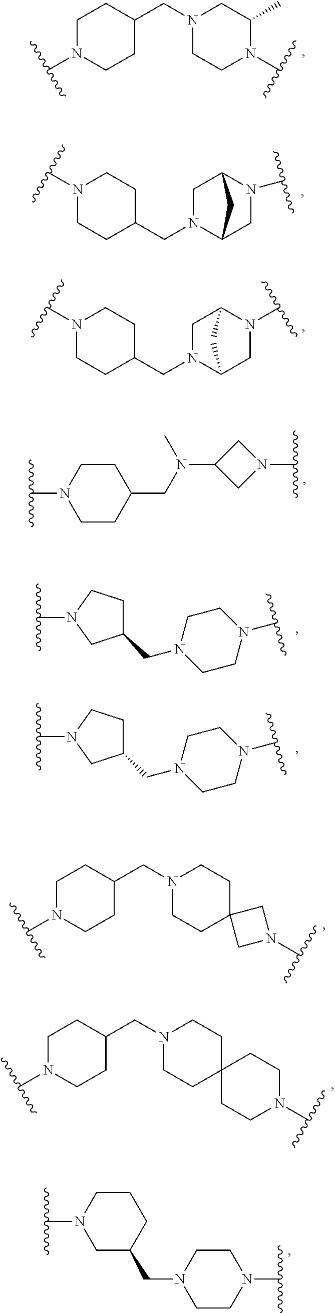

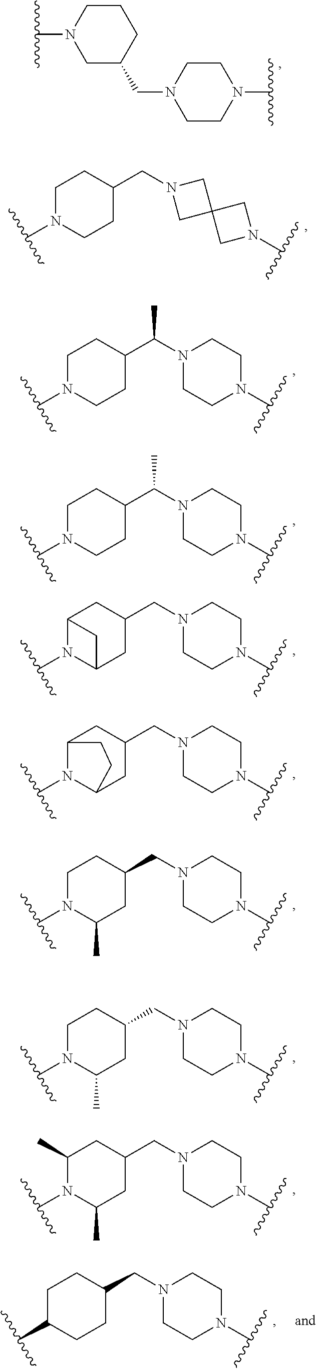

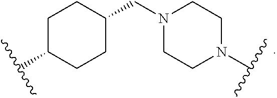 [SMILES: *N1CCN(C[C@H]2CC[C@@H](*)CC2)CC1]

537. The compound of any one of embodiments 1-527, wherein L is selected from:

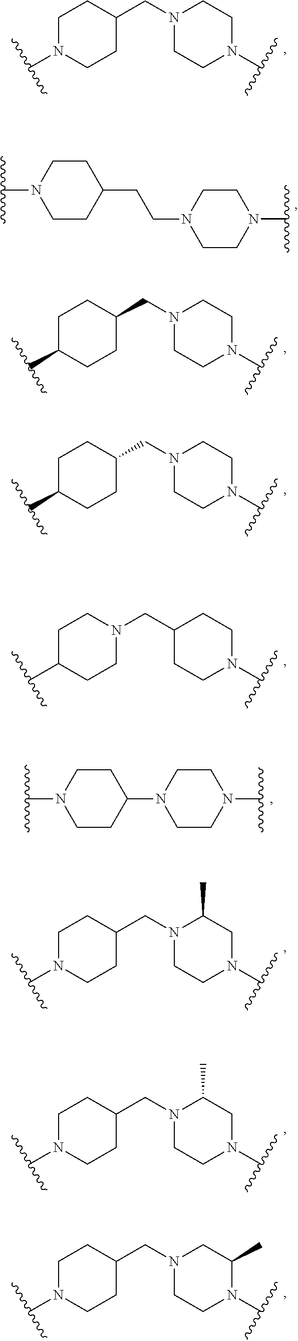

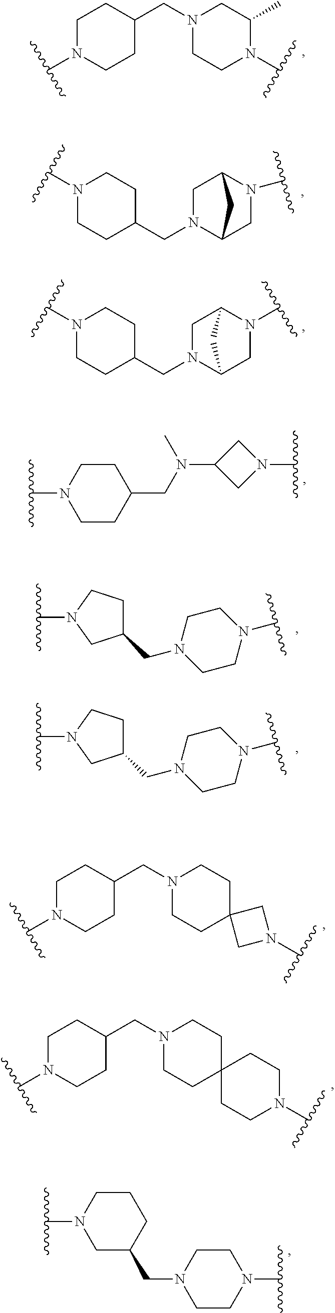

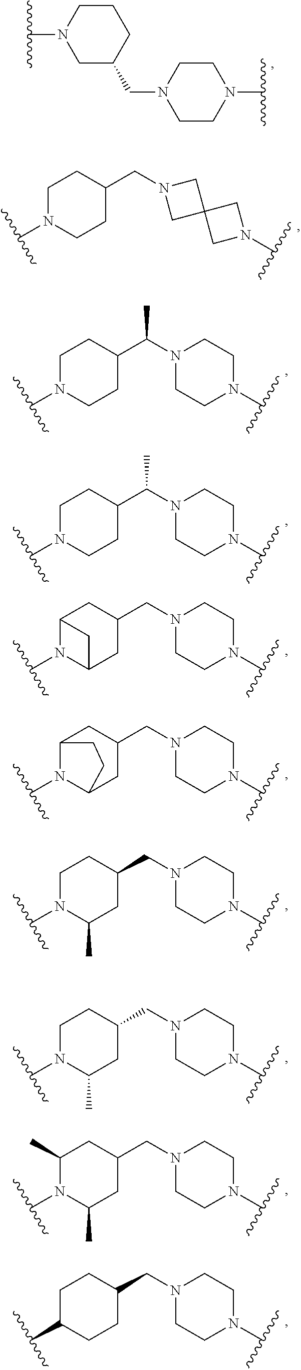

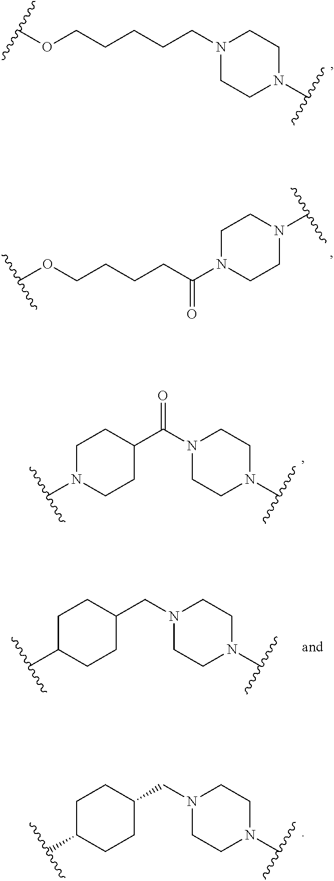 [SMILES: *OCCCCCN1CCN(*)CC1 ; *OCCCCC(=O)N1CCN(*)CC1 ; *N1CCC(C(=O)N2CCN(*)CC2)CC1 ; *C1CCC(CN2CCN(*)CC2)CC1 ; *N1CCN(C[C@H]2CC[C@@H](*)CC2)CC1]

538. The compound of any one of embodiments 1-527, wherein L is selected from:

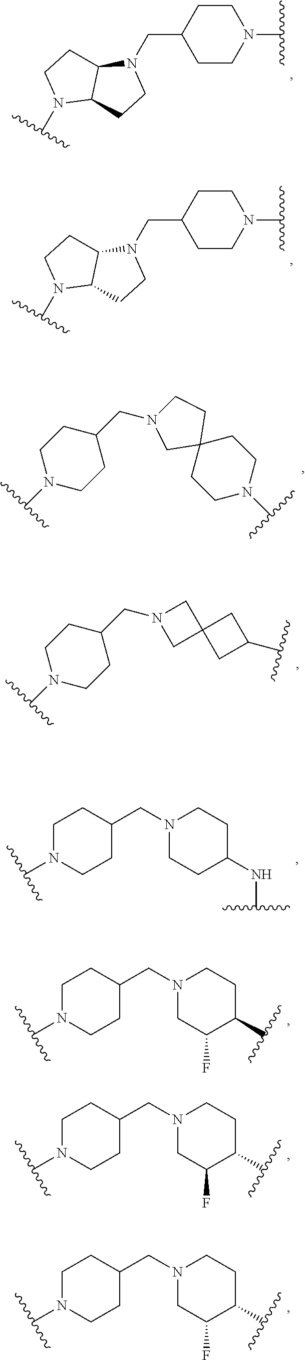

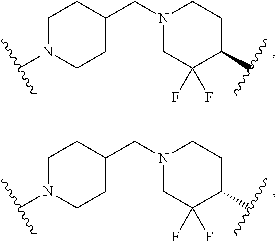 [SMILES: *[C@@H]1CCN(CC2CCN(*)CC2)CC1(F)F ; *[C@H]1CCN(CC2CCN(*)CC2)CC1(F)F]

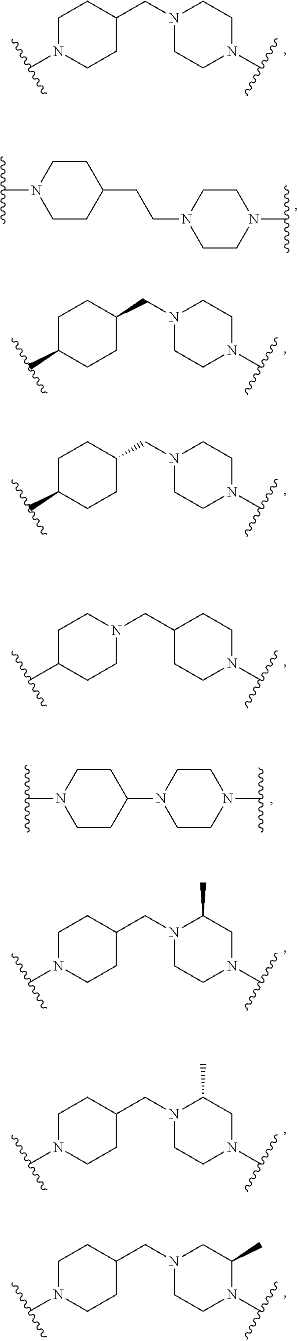

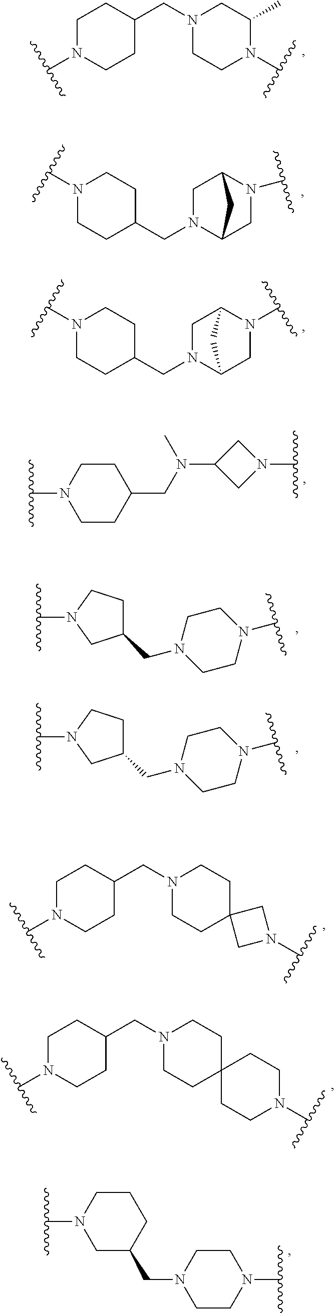

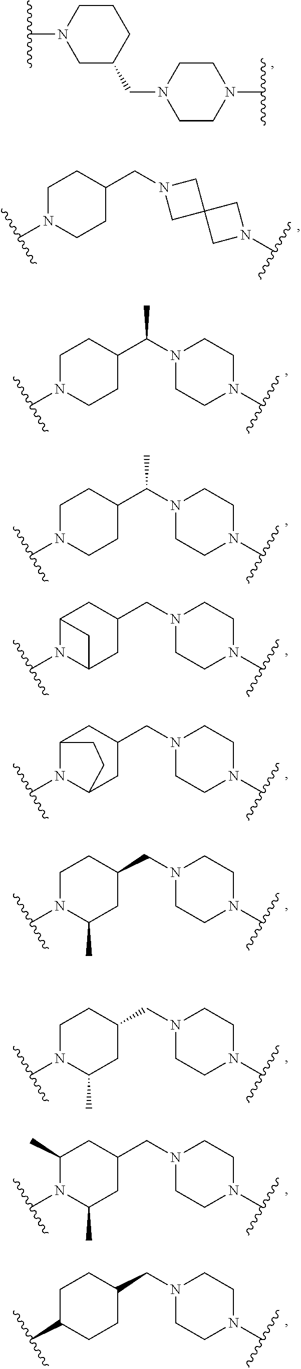

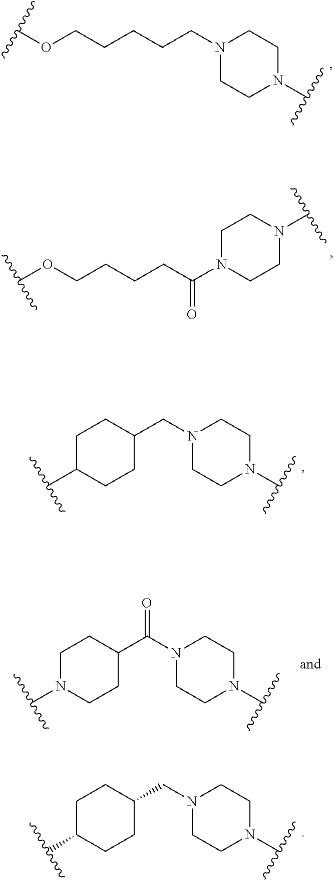 [SMILES: *OCCCCCN1CCN(*)CC1 ; *OCCCCC(=O)N1CCN(*)CC1 ; *C1CCC(CN2CCN(*)CC2)CC1 ; *N1CCC(C(=O)N2CCN(*)CC2)CC1 ; *N1CCN(C[C@H]2CC[C@@H](*)CC2)CC1]

539. The compound of any one of embodiments 1-527, wherein L is selected from:

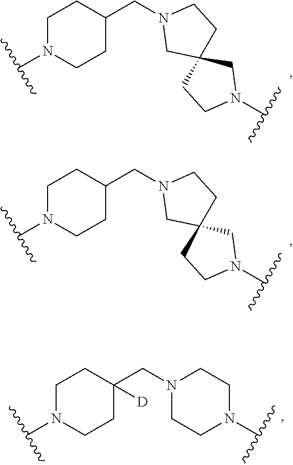 [SMILES: *N1CCC(CN2CC[C@]3(CCN(*)C3)C2)CC1 ; *N1CCC(CN2CC[C@@]3(CCN(*)C3)C2)CC1 ; *N1CCN(CC2([2H])CCN(*)CC2)CC1]

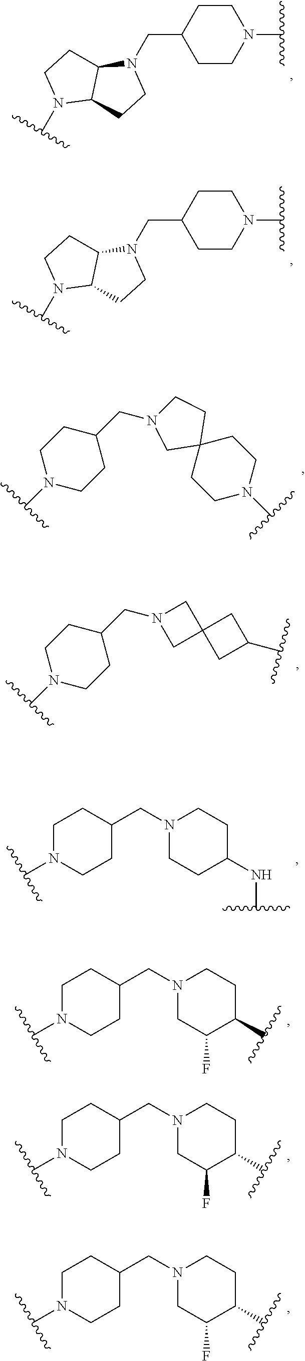

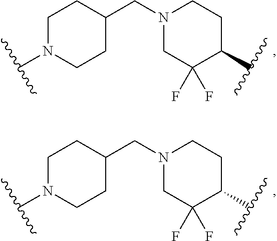 [SMILES: *[C@@H]1CCN(CC2CCN(*)CC2)CC1(F)F ; *[C@H]1CCN(CC2CCN(*)CC2)CC1(F)F]

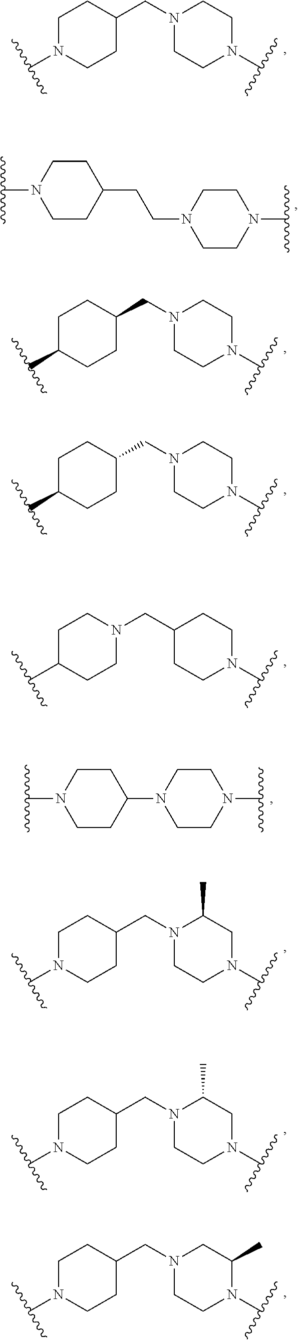

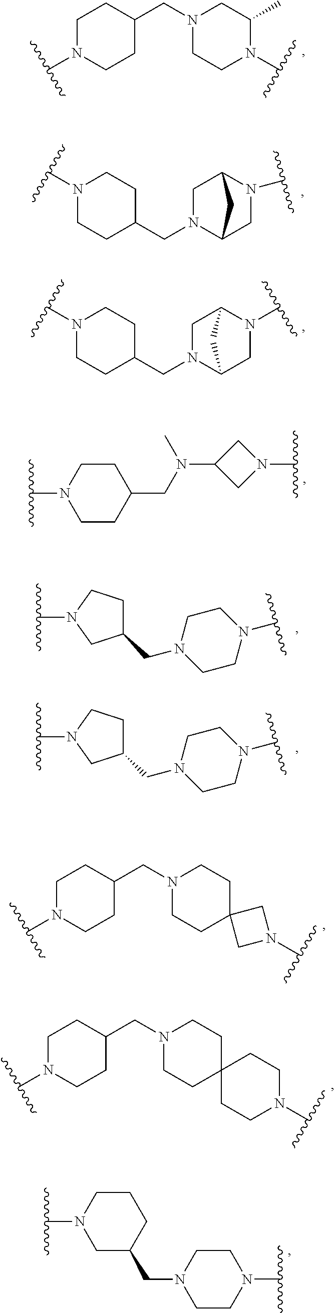

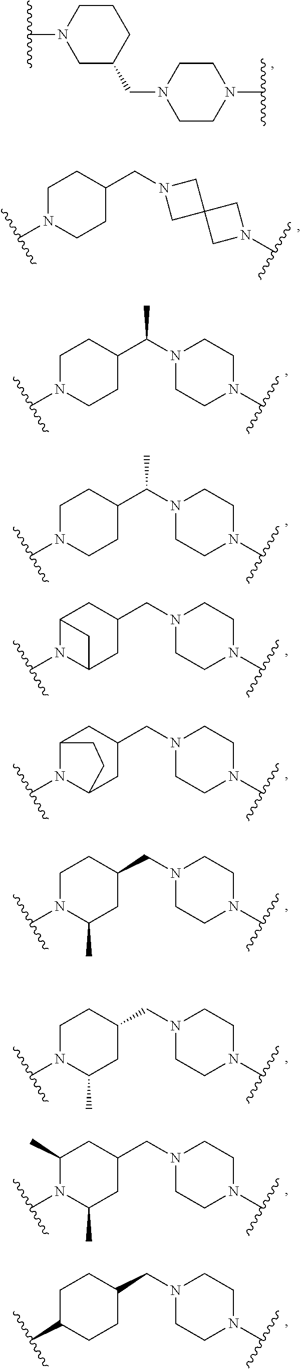

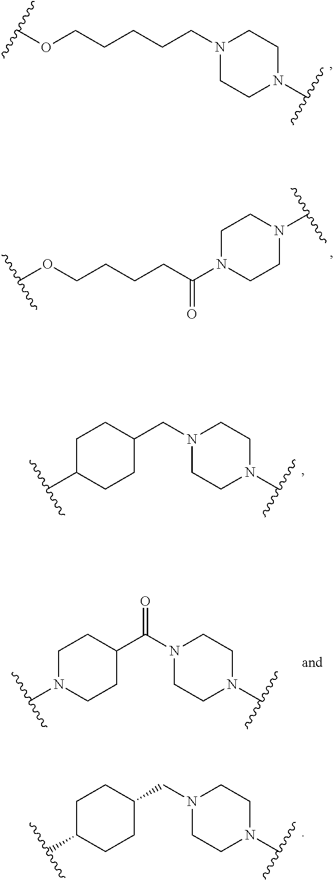 [SMILES: *OCCCCCN1CCN(*)CC1 ; *OCCCCC(=O)N1CCN(*)CC1 ; *C1CCC(CN2CCN(*)CC2)CC1 ; *N1CCC(C(=O)N2CCN(*)CC2)CC1 ; *N1CCN(C[C@H]2CC[C@@H](*)CC2)CC1]

540. The compound of any one of embodiments 1-527, wherein Lis

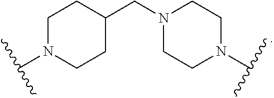 [SMILES: *N1CCC(CN2CCN(*)CC2)CC1]

541. The compound of any one of embodiments 1-527, wherein L is

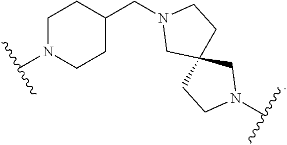 [SMILES: *N1CCC(CN2CC[C@]3(CCN(*)C3)C2)CC1]

542. The compound of any one of embodiments 1-527, wherein Lis

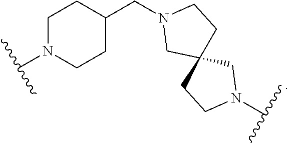 [SMILES: *N1CCC(CN2CC[C@@]3(CCN(*)C3)C2)CC1]

543. The compound of any one of embodiments 1-527, wherein L is

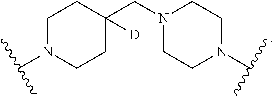 [SMILES: *N1CCN(CC2([2H])CCN(*)CC2)CC1]

544. The compound of any one of embodiments 1-527, wherein L is

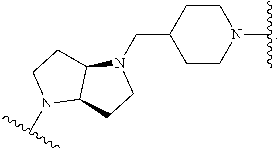 [SMILES: *N1CCC(CN2CC[C@@H]3[C@H]2CCN3*)CC1]

545. The compound of any one of embodiments 1-527, wherein L is

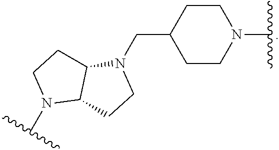 [SMILES: *N1CCC(CN2CC[C@H]3[C@@H]2CCN3*)CC1]

546. The compound of any one of embodiments 1-527, wherein L is

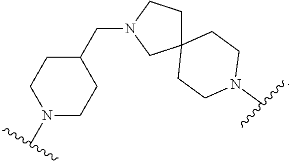 [SMILES: *N1CCC(CN2CCC3(CCN(*)CC3)C2)CC1]

547. The compound of any one of embodiments 1-527, wherein L is

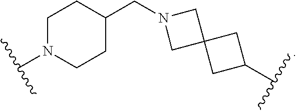 [SMILES: *C1CC2(C1)CN(CC1CCN(*)CC1)C2]

548. The compound of any one of embodiments 1-527, wherein L is

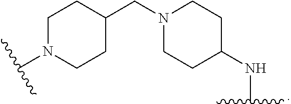 [SMILES: *NC1CCN(CC2CCN(*)CC2)CC1]

549. The compound of any one of embodiments 1-527, wherein Lis

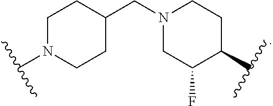 [SMILES: *[C@@H]1CCN(CC2CCN(*)CC2)C[C@H]1F]

550. The compound of any one of embodiments 1-527, wherein Lis

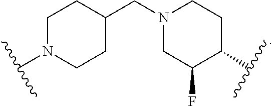 [SMILES: *[C@H]1CCN(CC2CCN(*)CC2)C[C@@H]1F]

551. The compound of any one of embodiments 1-527, wherein L is

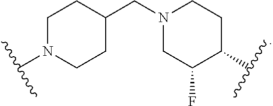 [SMILES: *[C@H]1CCN(CC2CCN(*)CC2)C[C@H]1F]

552. The compound of any one of embodiments 1-527, wherein L is

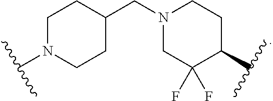 [SMILES: *[C@@H]1CCN(CC2CCN(*)CC2)CC1(F)F]

553. The compound of any one of embodiments 1-527, wherein L is

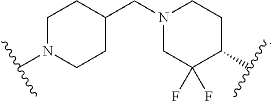 [SMILES: *[C@H]1CCN(CC2CCN(*)CC2)CC1(F)F]

554. The compound of any one of embodiments 1-527, wherein L is

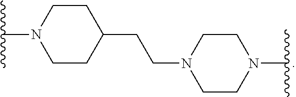 [SMILES: *N1CCC(CCN2CCN(*)CC2)CC1]

555. The compound of any one of embodiments 1-527, wherein Lis

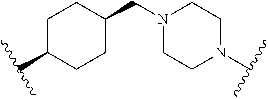 [SMILES: *N1CCN(C[C@H]2CC[C@@H](*)CC2)CC1]

556. The compound of any one of embodiments 1-527, wherein L is

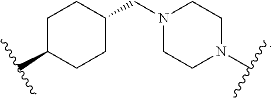 [SMILES: *N1CCN(C[C@H]2CC[C@H](*)CC2)CC1]

557. The compound of any one of embodiments 1-527, wherein L is

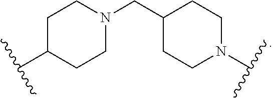 [SMILES: *C1CCN(CC2CCN(*)CC2)CC1]

558. The compound of any one of embodiments 1-527, wherein Lis

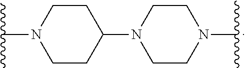 [SMILES: *N1CCC(N2CCN(*)CC2)CC1]

559. The compound of any one of embodiments 1-527, wherein L is

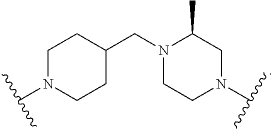 [SMILES: *N1CCC(CN2CCN(*)C[C@@H]2C)CC1]

560. The compound of any one of embodiments 1-527, wherein L is

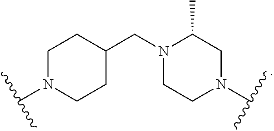 [SMILES: *N1CCC(CN2CCN(*)C[C@H]2C)CC1]

561. The compound of any one of embodiments 1-527, wherein L is

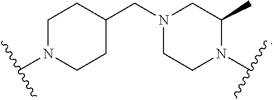 [SMILES: *N1CCC(CN2CCN(*)[C@H](C)C2)CC1]

562. The compound of any one of embodiments 1-527, wherein Lis

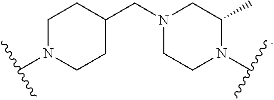 [SMILES: *N1CCC(CN2CCN(*)[C@@H](C)C2)CC1]

563. The compound of any one of embodiments 1-527, wherein Lis

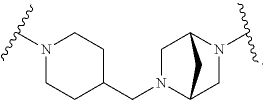 [SMILES: *N1CCC(CN2C[C@H]3C[C@@H]2CN3*)CC1]

564. The compound of any one of embodiments 1-527, wherein Lis

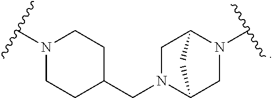 [SMILES: *N1CCC(CN2C[C@@H]3C[C@H]2CN3*)CC1]

565. The compound of any one of embodiments 1-527, wherein L is

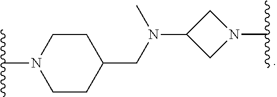 [SMILES: *N1CCC(CN(C)C2CN(*)C2)CC1]

566. The compound of any one of embodiments 1-527, wherein L is

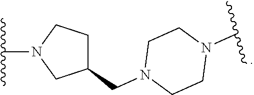 [SMILES: *N1CCN(C[C@@H]2CCN(*)C2)CC1]

567. The compound of any one of embodiments 1-527, wherein L is

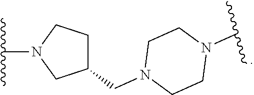 [SMILES: *N1CCN(C[C@H]2CCN(*)C2)CC1]

568. The compound of any one of embodiments 1-527, wherein L is

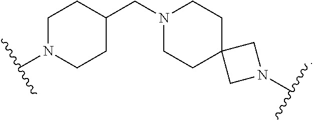 [SMILES: *N1CCC(CN2CCC3(CC2)CN(*)C3)CC1]

569. The compound of any one of embodiments 1-527, wherein L is

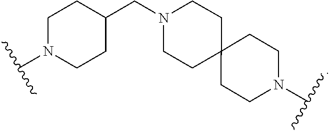 [SMILES: *N1CCC(CN2CCC3(CCN(*)CC3)CC2)CC1]

570. The compound of any one of embodiments 1-527, wherein Lis

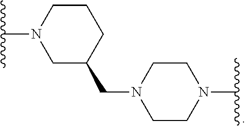 [SMILES: *N1CCN(C[C@@H]2CCCN(*)C2)CC1]

571. The compound of any one of embodiments 1-527, wherein Lis

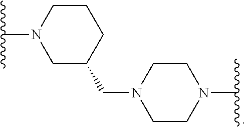 [SMILES: *N1CCN(C[C@H]2CCCN(*)C2)CC1]

572. The compound of any one of embodiments 1-527, wherein L is

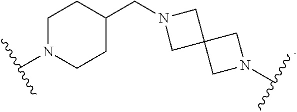 [SMILES: *N1CCC(CN2CC3(CN(*)C3)C2)CC1]

573. The compound of any one of embodiments 1-527, wherein L is

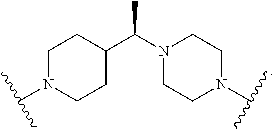 [SMILES: *N1CCC([C@@H](C)N2CCN(*)CC2)CC1]

574. The compound of any one of embodiments 1-527, wherein L is

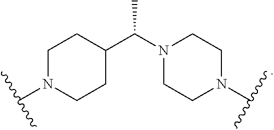 [SMILES: *N1CCC([C@H](C)N2CCN(*)CC2)CC1]

575. The compound of any one of embodiments 1-527, wherein Lis

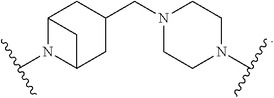 [SMILES: *N1CCN(CC2CC3CC(C2)N3*)CC1]

576. The compound of any one of embodiments 1-527, wherein L is

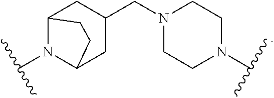 [SMILES: *N1CCN(CC2CC3CCC(C2)N3*)CC1]

577. The compound of any one of embodiments 1-527, wherein L is

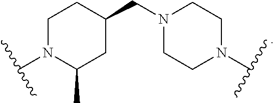 [SMILES: *N1CCN(C[C@@H]2CCN(*)[C@H](C)C2)CC1]

578. The compound of any one of embodiments 1-527, wherein Lis

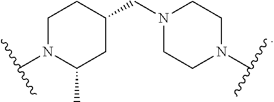 [SMILES: *N1CCN(C[C@H]2CCN(*)[C@@H](C)C2)CC1]

579. The compound of any one of embodiments 1-527, wherein Lis

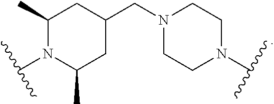 [SMILES: *N1CCN(CC2C[C@@H](C)N(*)[C@@H](C)C2)CC1]

580. The compound of any one of embodiments 1-527, wherein Lis

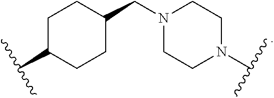

581. The compound of any one of embodiments 1-527, wherein L is

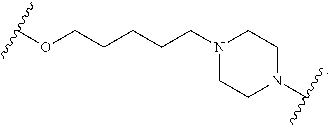 [SMILES: *OCCCCCN1CCN(*)CC1]

582. The compound of any one of embodiments 1-527, wherein L is

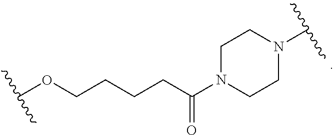 [SMILES: *OCCCCC(=O)N1CCN(*)CC1]

583. The compound of any one of embodiments 1-527, wherein Lis

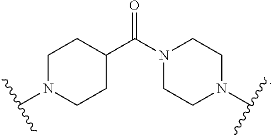 [SMILES: *N1CCC(C(=O)N2CCN(*)CC2)CC1]

584. The compound of any one of embodiments 1-527, wherein L is

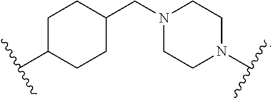 [SMILES: *C1CCC(CN2CCN(*)CC2)CC1]

585. The compound of any one of embodiments 1-527, wherein Lis

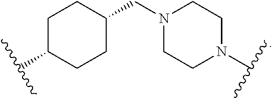 [SMILES: *N1CCN(C[C@H]2CC[C@@H](*)CC2)CC1]

586. The compound of any one of embodiments 1-585, wherein LBM is selected from:

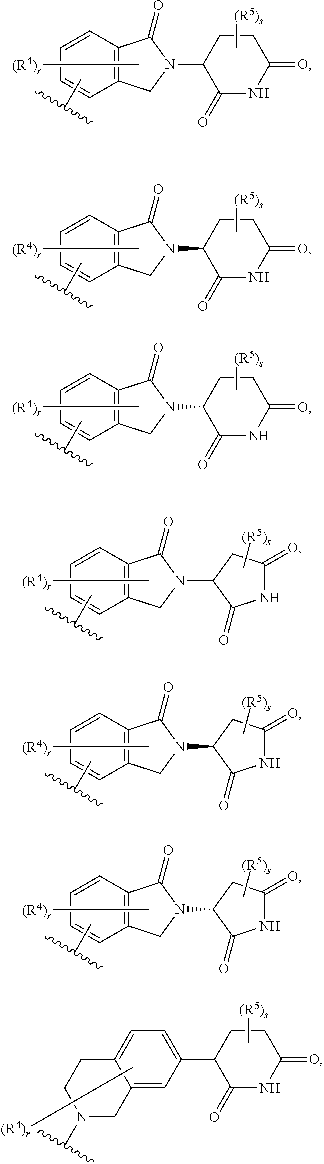

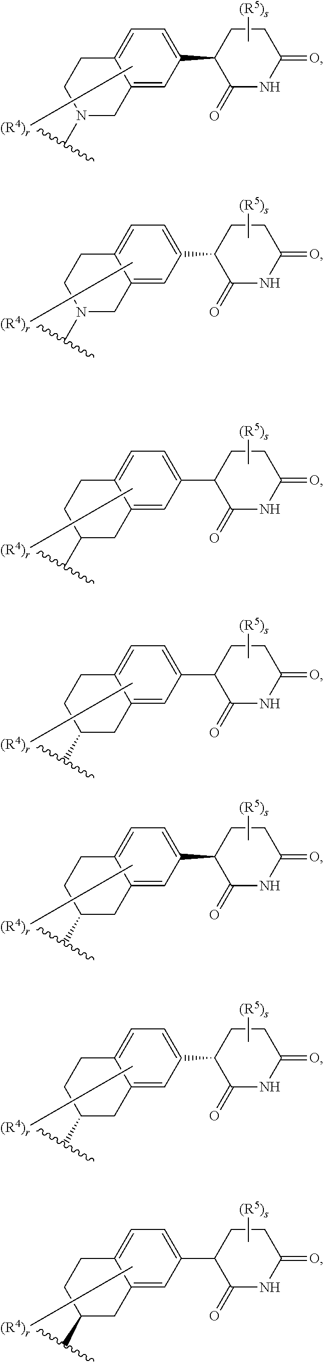

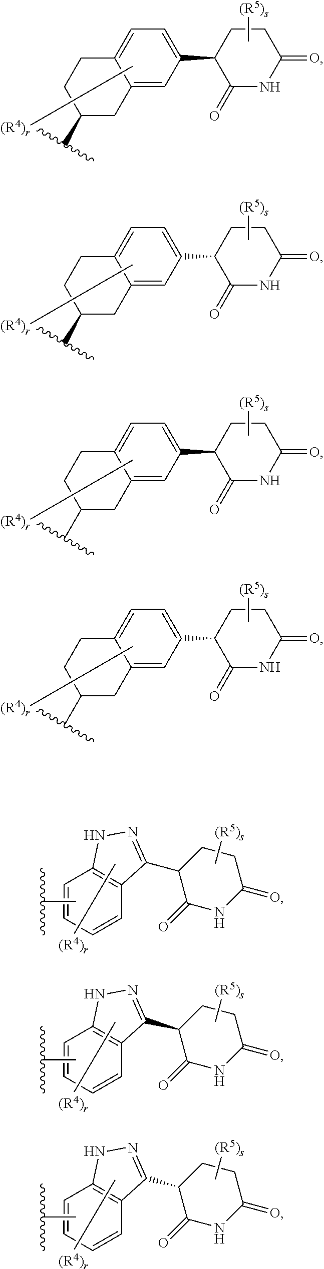

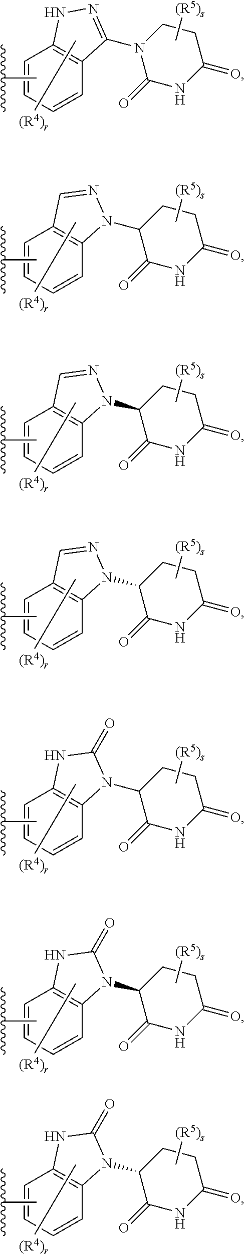

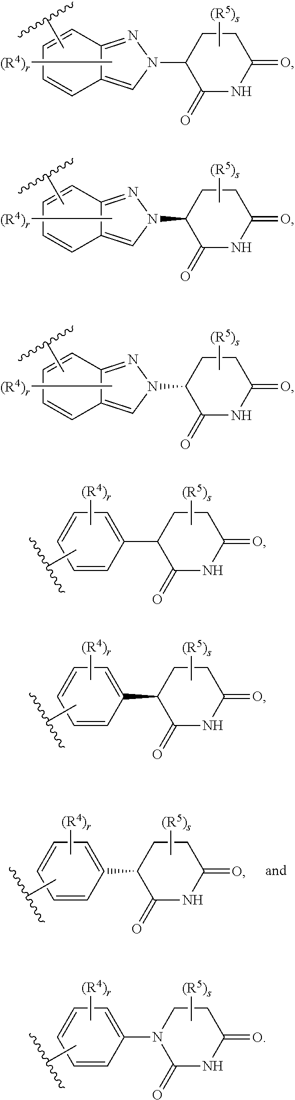

587. The compound of any one of embodiments 1-585, wherein LBM is selected from:

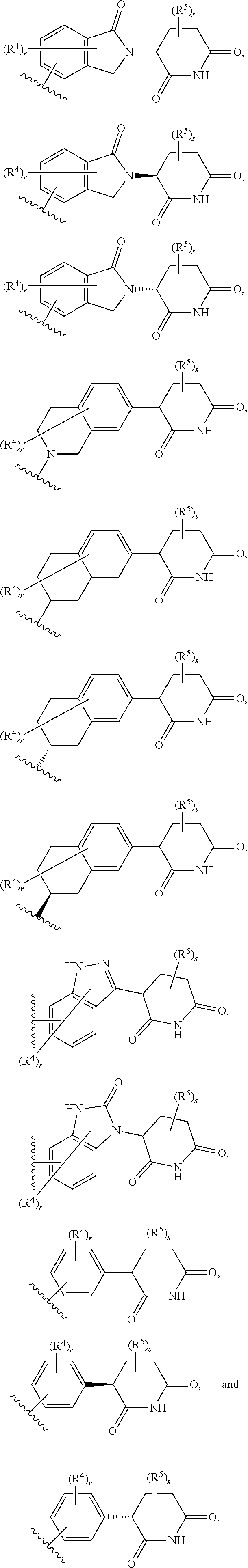

588. The compound of any one of embodiments 1-585, wherein LBM is selected from

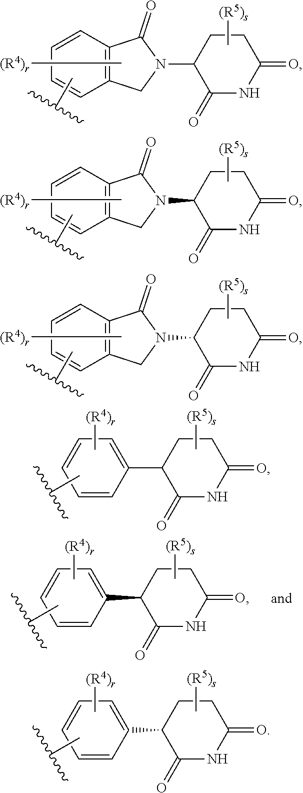

589. The compound of any one of embodiments 1-588, wherein each R⁴ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R) OR, —OC(O)NR₂, —N(R)C(O) OR, —N(R)C(O)R, —N(R)C(O)NR₂, —N(R)S(O)₂NR₂ and —N(R)S(O)₂R, wherein R is H or a C₁₋₆ aliphatic chain.

590. The compound of any one of embodiments 1-588, wherein each R⁴ is independently selected from -Me, -Et, —F, —Cl, —CF₃, —CN, —OH, —OMe, —NH₂, —NHMe and —NMe₂.

591. The compound of any one of embodiments 1-590, wherein each R⁵ is independently selected from deuterium, C₁₋₆ aliphatic chain substituted with 0-3 instances of halo, halogen, —CN, —OR, —NR₂, —S(O) ¿R, —S(O)₂NR₂, —S(O)R, —S(O)NR₂, —S(O)(NR)R, —C(O)NR₂, —C(O)N(R) OR, —OC(O)NR₂, —N(R)C(O) OR, —N(R)C(O)R, —N(R)C(O)NR₂, —N(R)S(O)₂NR₂ and —N(R)S(O)₂R, wherein R is H or a C₁₋₆ aliphatic chain.

592. The compound of any one of embodiments 1-590, wherein each R⁵ is independently selected from -Me, -Et, —F, —Cl, —CF3, —CN, —OH, —OMe, —NH2, —NHMe and —NMe2.

593. The compound of any one of embodiments 1-590, wherein each R⁵ is independently selected from -Me and —F.

594. The compound of any one of embodiments 1-593, wherein r is 0, 1 or 2.

595. The compound of any one of embodiments 1-593, wherein r is 0.

596. The compound of any one of embodiments 1-593, wherein r is 1.

597. The compound of any one of embodiments 1-593, wherein r is 2.

598. The compound of any one of embodiments 1-597, wherein s is 0, 1 or 2.

599. The compound of any one of embodiments 1-597, wherein s is 0.

600. The compound of any one of embodiments 1-597, wherein s is 1.

601. The compound of any one of embodiments 1-597, wherein s is 2.

602. The compound of any one of embodiments 1-585, wherein LBM is selected from

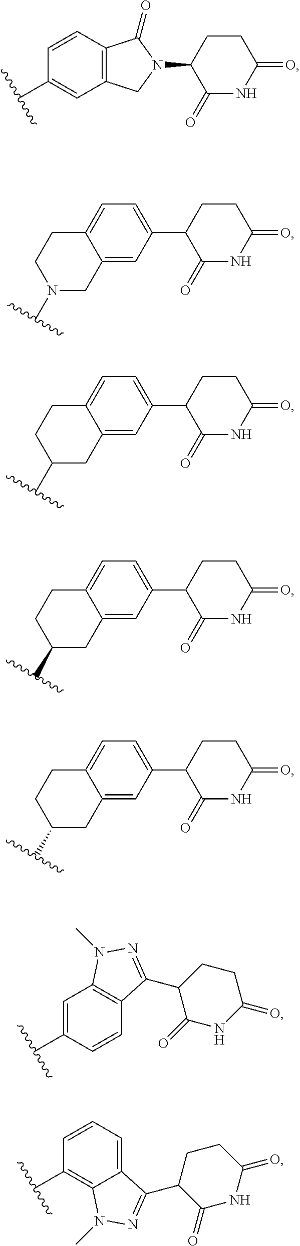

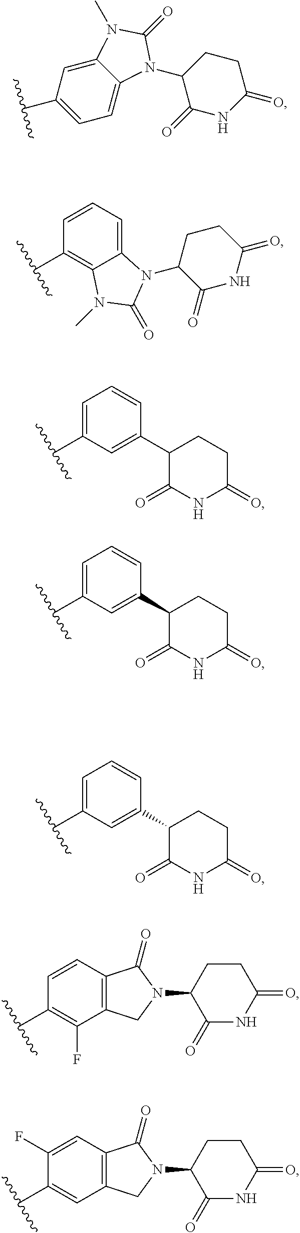

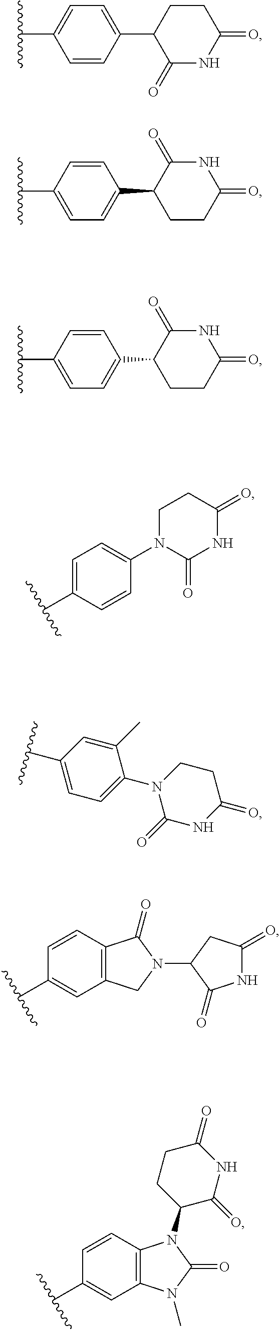

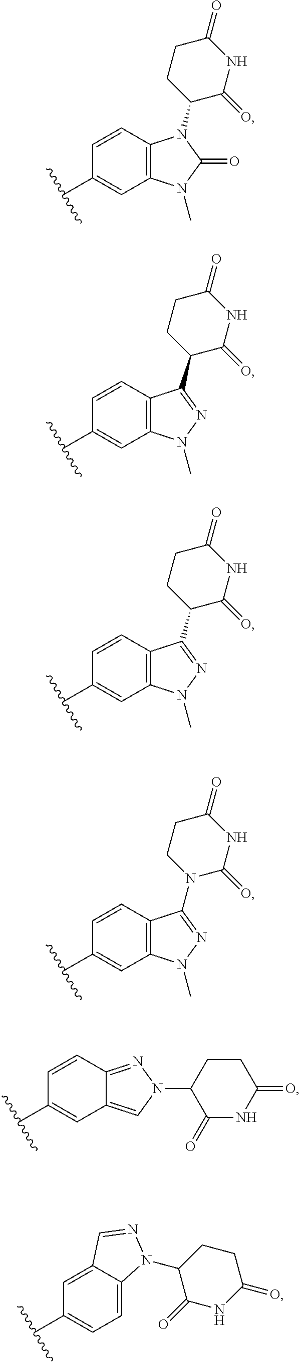

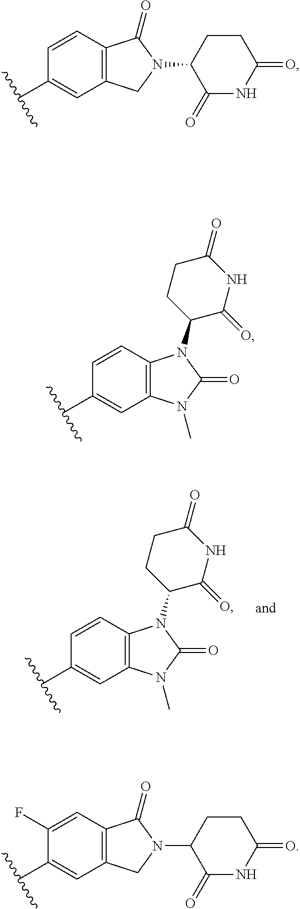 [SMILES: *c1ccc2c(c1)CN([C@@H]1CCC(=O)NC1=O)C2=O ; *c1ccc2c(c1)n(C)c(=O)n2[C@H]1CCC(=O)NC1=O ; *c1ccc2c(c1)n(C)c(=O)n2[C@@H]1CCC(=O)NC1=O ; *c1cc2c(cc1F)C(=O)N(C1CCC(=O)NC1=O)C2]

603. The compound of any one of embodiments 1-585, wherein LBM is selected from:

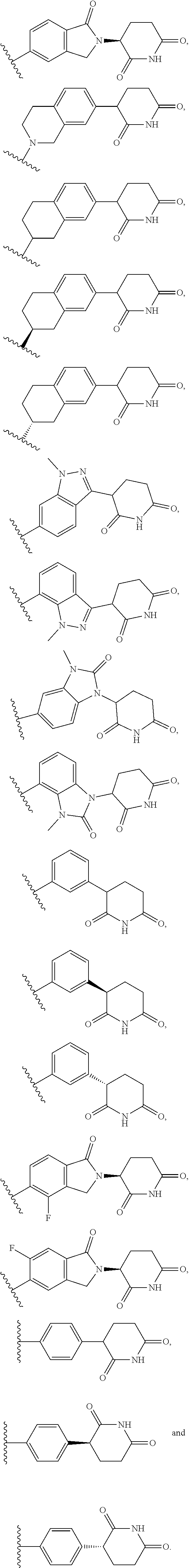

604. The compound of any one of embodiments 1-585, wherein LBM is selected from

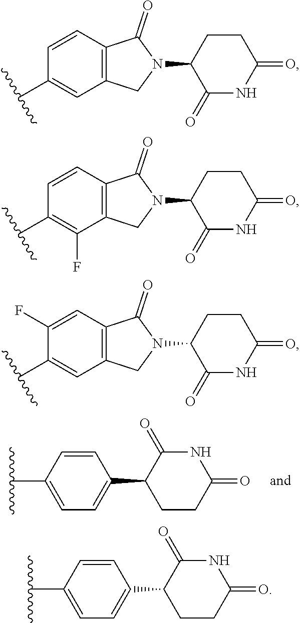

605. The compound of any one of embodiments 1-585, wherein LBM is selected from

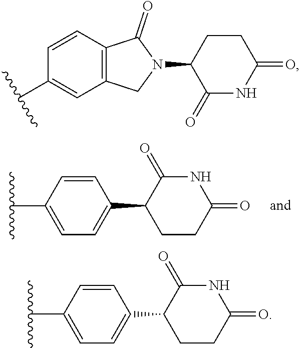

606. The compound of any one of embodiments 1-585, wherein LBM is

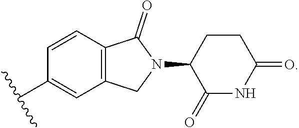 [SMILES: *c1ccc2c(c1)CN([C@H]1CCC(=O)NC1=O)C2=O]

607. The compound of any one of embodiments 1-585, wherein LBM is

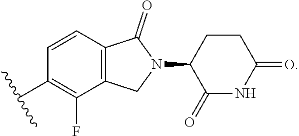 [SMILES: *c1ccc2c(c1F)CN([C@H]1CCC(=O)NC1=O)C2=O]

608. The compound of any one of embodiments 1-585, wherein LBM is

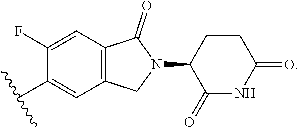 [SMILES: *c1cc2c(cc1F)C(=O)N([C@H]1CCC(=O)NC1=O)C2]

609. The compound of any one of embodiments 1-585, wherein LBM is

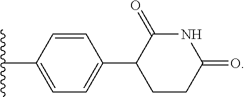 [SMILES: *c1ccc(C2CCC(=O)NC2=O)cc1]

610. The compound of any one of embodiments 1-585, wherein LBM is

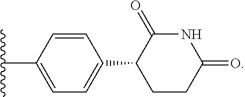 [SMILES: *c1ccc([C@H]2CCC(=O)NC2=O)cc1]

611. The compound of any one of embodiments 1-585, wherein LBM is

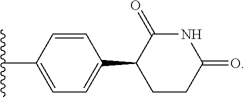 [SMILES: *c1ccc([C@@H]2CCC(=O)NC2=O)cc1]

612. The compound of any one of embodiments 1-585, wherein LBM is

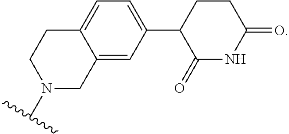 [SMILES: *N1CCc2ccc(C3CCC(=O)NC3=O)cc2C1]

613. The compound of any one of embodiments 1-585, wherein LBM is

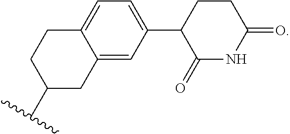 [SMILES: *C1CCc2ccc(C3CCC(=O)NC3=O)cc2C1]

614. The compound of any one of embodiments 1-585, wherein LBM is

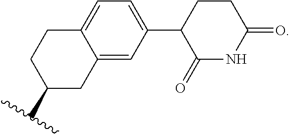 [SMILES: *[C@H]1CCc2ccc(C3CCC(=O)NC3=O)cc2C1]

615. The compound of any one of embodiments 1-585, wherein LBM is

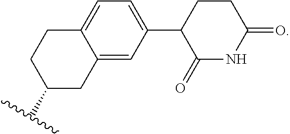 [SMILES: *[C@@H]1CCc2ccc(C3CCC(=O)NC3=O)cc2C1]

616. The compound of any one of embodiments 1-585, wherein LBM is

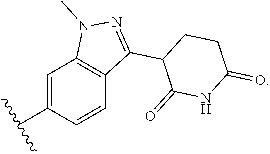 [SMILES: *c1ccc2c(C3CCC(=O)NC3=O)nn(C)c2c1]

617. The compound of any one of embodiments 1-585, wherein LBM is

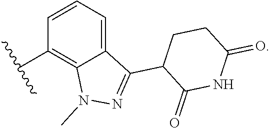 [SMILES: *c1cccc2c(C3CCC(=O)NC3=O)nn(C)c12]

618. The compound of any one of embodiments 1-585, wherein LBM is

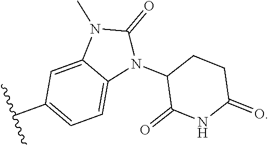 [SMILES: *c1ccc2c(c1)n(C)c(=O)n2C1CCC(=O)NC1=O]

619. The compound of any one of embodiments 1-585, wherein LBM is

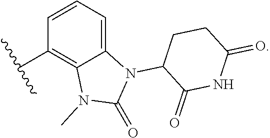 [SMILES: *c1cccc2c1n(C)c(=O)n2C1CCC(=O)NC1=O]

620. The compound of any one of embodiments 1-585, wherein LBM is

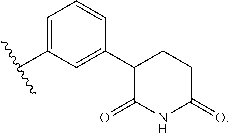 [SMILES: *c1cccc(C2CCC(=O)NC2=O)c1]

621. The compound of any one of embodiments 1-585, wherein LBM is

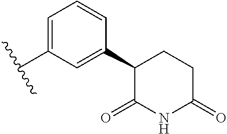 [SMILES: *c1cccc([C@H]2CCC(=O)NC2=O)c1]

622. The compound of any one of embodiments 1-585, wherein LBM is

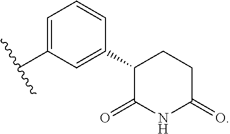 [SMILES: *c1cccc([C@@H]2CCC(=O)NC2=O)c1]

623. The compound of any one of embodiments 1-585, wherein LBM is

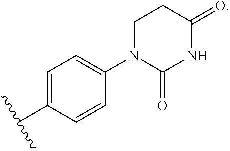 [SMILES: *c1ccc(N2CCC(=O)NC2=O)cc1]

624. The compound of any one of embodiments 1-585, wherein LBM is

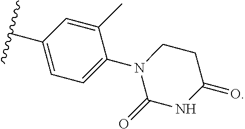 [SMILES: *c1ccc(N2CCC(=O)NC2=O)c(C)c1]

625. The compound of any one of embodiments 1-585, wherein LBM is

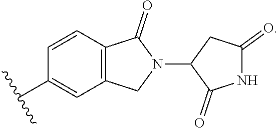 [SMILES: *c1ccc2c(c1)CN(C1CC(=O)NC1=O)C2=O]

626. The compound of any one of embodiments 1-585, wherein LBM is

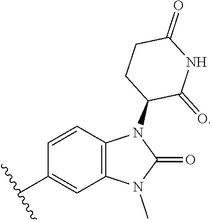 [SMILES: *c1ccc2c(c1)n(C)c(=O)n2[C@H]1CCC(=O)NC1=O]

627. The compound of any one of embodiments 1-585, wherein LBM is

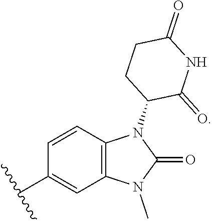 [SMILES: *c1ccc2c(c1)n(C)c(=O)n2[C@@H]1CCC(=O)NC1=O]

628. The compound of any one of embodiments 1-585, wherein LBM is

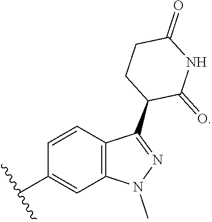 [SMILES: *c1ccc2c([C@H]3CCC(=O)NC3=O)nn(C)c2c1]

629. The compound of any one of embodiments 1-585, wherein LBM is

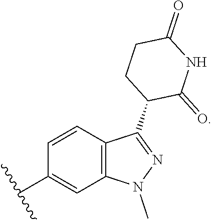 [SMILES: *c1ccc2c([C@@H]3CCC(=O)NC3=O)nn(C)c2c1]

630. The compound of any one of embodiments 1-585, wherein LBM is

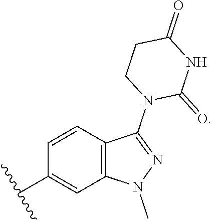 [SMILES: *c1ccc2c(N3CCC(=O)NC3=O)nn(C)c2c1]

631. The compound of any one of embodiments 1-585, wherein LBM is

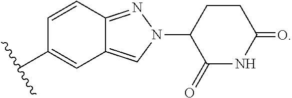 [SMILES: *c1ccc2nn(C3CCC(=O)NC3=O)cc2c1]

632. The compound of any one of embodiments 1-585, wherein LBM is

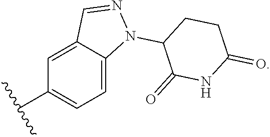 [SMILES: *c1ccc2c(cnn2C2CCC(=O)NC2=O)c1]

633. The compound of any one of embodiments 1-585, wherein LBM is

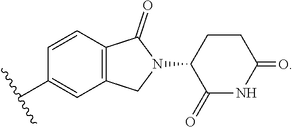 [SMILES: *c1ccc2c(c1)CN([C@@H]1CCC(=O)NC1=O)C2=O]

634. The compound of any one of embodiments 1-585, wherein LBM is

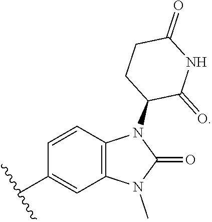 [SMILES: *c1ccc2c(c1)n(C)c(=O)n2[C@H]1CCC(=O)NC1=O]

635. The compound of any one of embodiments 1-585, wherein LBM is

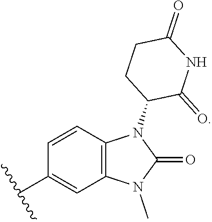 [SMILES: *c1ccc2c(c1)n(C)c(=O)n2[C@@H]1CCC(=O)NC1=O]

636. The compound of any one of embodiments 1-585, wherein LBM is

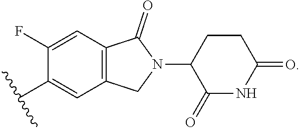 [SMILES: *c1cc2c(cc1F)C(=O)N(C1CCC(=O)NC1=O)C2]

637. The compound of any one of embodiments 1-636, wherein each R¹ is independently selected from deuterium, C₁₋₆ aliphatic chain substituted with 0-3 instances of halo, halogen, —CN, —SR, —OR, —NR₂, —S(O)₂R, —S(O)₂NR₂, —S(O)R, —S(O)NR₂, —S(O)(NR)R, —C(O) OR, —C(O)NR₂, —C(O)N(R) OR, —OC(O)NR₂, —N(R)C(O) OR, —N(R)C(O)R, —N(R)C(O)NR₂, —N(R)S(O)₂NR₂ and —N(R)S(O)₂R, wherein R is H or a C₁₋₆ aliphatic chain substituted with 0-3 instances of halo.

638. The compound of any one of embodiments 1-636, wherein each R is independently selected from -Me, -Et, —F, —Cl, —SCF₃, —OCF₃, —CF₃, —CN, —OH, —OMe, —NH₂, —NHMe and —NMe₂.

639. The compound of any one of embodiments 1-636, wherein each R¹ is independently selected from -Me and —F,

640. The compound of any one of embodiments 1-639, wherein each R² is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O) OR, —C(O)NR₂, —C(O)N(R) OR, —OC(O)NR₂, —N(R)C(O) OR, —N(R)C(O)R, —N(R)C(O)NR₂, —N(R)S(O)₂NR₂ and —N(R)S(O)₂R, wherein R is H or a C₁₋₆ aliphatic chain.

641. The compound of any one of embodiments 1-639, wherein each R² is independently selected from -Me, -Et, —F, —Cl, —CF₃, —CN, —OH, —OMe, —NH₂, —NHMe and —NMe₂.

542. The compound of any one of embodiments 1-639, wherein each R² is independently selected from —F, —Cl and —CF₃.

643. The compound of any one of embodiments 1-642, wherein m is 0, 1 or 2.

644. The compound of any one of embodiments 1-642, wherein m is 0.

645. The compound of any one of embodiments 1-642, wherein m is 1.

646. The compound of any one of embodiments 1-642, wherein m is 2.

647. The compound of any one of embodiments 1-646, wherein n is 0, 1 or 2.

648. The compound of any one of embodiments 1-646, wherein n is 0.

649. The compound of any one of embodiments 1-646, wherein n is 1.

650. The compound of any one of embodiments 1-646, wherein n is 2.

651. The compound of any one of embodiments 1-650, wherein each R³ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O) OR, —C(O)NR₂, —C(O)N(R) OR, —OC(O)NR₂, —N(R)C(O) OR, —N(R)C(O)R, —N(R)C(O)NR₂, —N(R)S(O)₂NR₂ and —N(R)S(O)₂R, wherein R is H or a C₁₋₆ aliphatic chain or two R³ groups are taken together to form

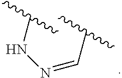 [SMILES: */C=N\N*]

652. The compound of any one of embodiments 1-650, wherein each R³ is independently selected from -Me, -Et, —F, —Cl, —CF₃, —CO₂H, —CN, —OH, —OMe, —NH₂, —NHMe and —NMe₂ or two R³ groups are taken together to form

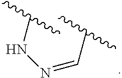 [SMILES: */C=N\N*]

653. The compound of any one of embodiments 1-650, wherein each R³ is independently selected from —F, —OH, and —CO₂H.

654. The compound of any one of embodiments 1-650, wherein each R³ is independently selected from -Me, —F, —OH, and —CO₂H.

655. The compound of any one of embodiments 1-650, wherein each R³ is independently selected from —F and —OH.

656. The compound of any one of embodiments 1-650, wherein each R³ is independently selected from -Me and —F.

657. The compound of any one of embodiments 1-650, wherein two R³ groups are taken together to form

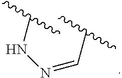 [SMILES: */C=N\N*]

658. The compound of any one of embodiments 1-657, wherein p is 0, 1 or 2.

659. The compound of any one of embodiments 1-657, wherein p is 0.

660. The compound of any one of embodiments 1-657, wherein p is 1.

661. The compound of any one of embodiments 1-657, wherein p is 2.

662. The compound of any one of embodiments 1-661, wherein each R⁶ is independently selected from -Me, -Et, —F, —Cl, —CF₃, —CO₂H, —CN, —OH, —OMe, —NH₂, —NHMe and —NMe₂

663. The compound of any one of embodiments 1-661, wherein each R⁶ is independently selected from -Me, and —F.

664. The compound of any one of embodiments 1-663, wherein t is 0, 1 or 2.

665. The compound of any one of embodiments 1-663, wherein t is 0.

666. The compound of any one of embodiments 1-663, wherein t is 1.

667. The compound of any one of embodiments 1-663, wherein t is 2.

668. The compound of any one of embodiments 1-667, wherein the compound is a compound shown in Table 1 or a pharmaceutically acceptable salt thereof.

669. A compound shown in Table 2 or a pharmaceutically acceptable salt thereof.

670. A pharmaceutical composition comprising a compound of any one of embodiments 1-669, or a pharmaceutically acceptable salt thereof, and a pharmaceutically acceptable carrier, adjuvant, or diluent.

671. A method of inhibiting ERα signaling in a sample, e.g., in vivo or in vitro, by contacting ERα with a compound of any one of embodiments 1-669, or a pharmaceutically acceptable salt thereof, or a pharmaceutical composition of embodiment 670.

672. The method of embodiment 671, wherein the inhibiting of ERα signaling comprises reducing the signaling activity of ERα by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

673. The method of embodiment 671, wherein the inhibiting of ERα signaling comprises reducing the signaling activity of ERα by at least 1-fold, 1.5-fold, 2-fold, 3-fold, 5-fold, 10-fold, 20-fold, 30-fold, 50-fold, 100-fold, or more, e.g., relative to a reference standard.

674. A method of treating an ERα-mediated disorder in a patient in need thereof, comprising administering to the patient a compound of any one of embodiments 1-669, or a pharmaceutically acceptable salt thereof, or a composition of embodiment 670.

675. The method of embodiment 674, wherein the ER-mediated disorder is associated with estrogen receptor accumulation and aggregation.

676. The method of any of embodiments 674 or 675, wherein the ERα-mediated disorder is cancer or a neoplasia associated with estrogen receptor accumulation and aggregation.

677. The method of embodiment 676, wherein the ERα-mediated disorder is a disorder mediated by a ERα containing a D544G, Y543S, or L542R mutation.

678. The method of any one of embodiments 674-677, wherein the ERα-mediated disorder is a disorder described herein.

679 The method of any one of embodiments 674-678, wherein the method comprises the steps of:
(i) identifying a subject in need of such treatment;
(ii) providing a disclosed compound, or a pharmaceutically acceptable salt thereof; and
(iii) administering said provided compound in a therapeutically effective amount to treat, suppress and/or prevent the disease state or condition in a subject in need of such treatment.

680. The method of any one of embodiments 674-679, wherein the ERα-mediated disorder is breast cancer or uterine cancer.

681 The method of embodiment 680, wherein the breast cancer is selected from the group consisting of ER+ breast cancer, ER+/HER2-breast cancer, ER+ advanced/metastatic breast cancer, and ER+/HER2-advanced/metastatic breast cancer.

682. The method of any one of embodiments 674-681, wherein the ERα-mediated disorder is endometriosis.

683. A compound or pharmaceutically acceptable salt, solvate, stereoisomer, or tautomer thereof, according to any one of embodiments 1-669, or a pharmaceutically acceptable salt thereof, or a pharmaceutical composition of embodiment 670, for use as a medicament.

684. A compound or pharmaceutically acceptable salt, solvate, stereoisomer, or tautomer thereof, according to any one of embodiments 1-669, or a pharmaceutically acceptable salt thereof, or a pharmaceutical composition of embodiment 670, for use in the treatment of an ERα-mediated disorder.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### General Methods

The parameters used for the HPLC measurement in the synthesis procedures below are as follows: Mobile Phase: 2.75ML/4LTFA in water (solvent A) and 2.5ML/4LTFA in acetonitrile (solvent B), using the elution gradient 10%-80% (solvent B) over 6 minutes and holding at 80% for 2 minutes at a flow rate of 1.2 ml/min; Column: Ultimate C18 3.0*50 mm, 3 um Wavelength: UV220 nm, 215 nm, 254 nm; Column temperature: 40° C.

The parameters for the LCMS (acidic) measurement in the synthesis procedures below are one of the following: (1) Mobile Phase: 1.5ML/4LTFA in water (solvent A) and 0.75ML/4LTFA in acetonitrile (solvent B), using the elution gradient 5%-95% (solvent B) over 0.7 minutes and holding at 95%; for 0.4 minutes at a flow rate of 1.5 ml/min; Column: MerckChromolith®FlashRP-183*25 mm, 3 um; Wavelength: UV 220 nm, 254 nm; Column temperature: 50° C.; MS ionization: ESI. (2) Mobile Phase: 1.5ML/4LTFA in water (solvent A) and 0.75ML/4LTFA in acetonitrile (solvent B), using the elution gradient 10%-80% (solvent B) over 3 minutes and holding at 80%; for 0.5 minutes at a flow rate of 0.8 ml/min; column: Xtimate C18 2.1*30 mm, 3 um.

The parameters for the LCMS (basic) measurement in the synthesis procedures below are as follows: Mobile Phase: 0.8 mL/4 L NH3·H2O in water (solvent A) and acetonitrile (solvent B), using the elution gradient 30%-90% (solvent B) over 2 minutes and holding at 90%; for 0.48 minutes at a flow rate of 1.2 ml/min; Column: Xbridge Shield RP-18.5 um, 2.1*50 mm; Wavelength: UV 220 nm & 254 nm; Column temperature: 50° C.; MS ionization:ESI. Wavelength: UV 220 nm & 254 nm; Column temperature: 50° C.; MS ionization:ESI.

### List of Abbreviations

aq: aqueous
Ac: acetyl
ACN or MeCN: acetonitrile
AmF: ammonium formate
anhyd.: anhydrous
BINAP: (±)-2,2′-Bis(diphenylphosphino)-1,1′-binaphthalene
Bn: Benzyl
conc.: concentrated
DBU: 1,8-Diazabicyclo[5.4.0]undec-7-ene
DCE: Dichloroethane
DCM: Dichloromethane
DIPEA: Diisopropylamine
DMF: N,N-dimethylformamide
DMP: Dess-Martin periodinane
DMPU: N,N′-Dimethylpropyleneurea
DMSO: dimethylsulfoxide
DIPEA: diisopropylethylamine
EA or EtOAc: ethyl acetate
EDCI, EDC, or EDAC: 1-Ethyl-3-(3-dimethylaminopropyl)carbodiimide
equiv or eq: molar equivalents
Et: ethyl
HATU: 1-[Bis(dimethylamino)methylene]-1H-1,2,3-triazolo[4,5-b]pyridinium 3-oxid Hexafluorophosphate
HPLC: high pressure liquid chromatography
LCMS or LC-MS: liquid chromatography-mass spectrometry
Ms: methanesulfonyl
NBS: N-bromosuccinimide
NMR: nuclear magnetic resonance
PE: petroleum ether
PMB: p-methoxybenzyl
rt or RT: room temperature
sat: saturated
TBS: tert-butyldimethylsilyl
TEA: triethylamine
Tf: trifluoromethanesulfonate
TFA: trifluoroacetic acid
THF: tetrahydrofuran
TLC: thin layer chromatography
Tol: toluene
UV: ultraviolet

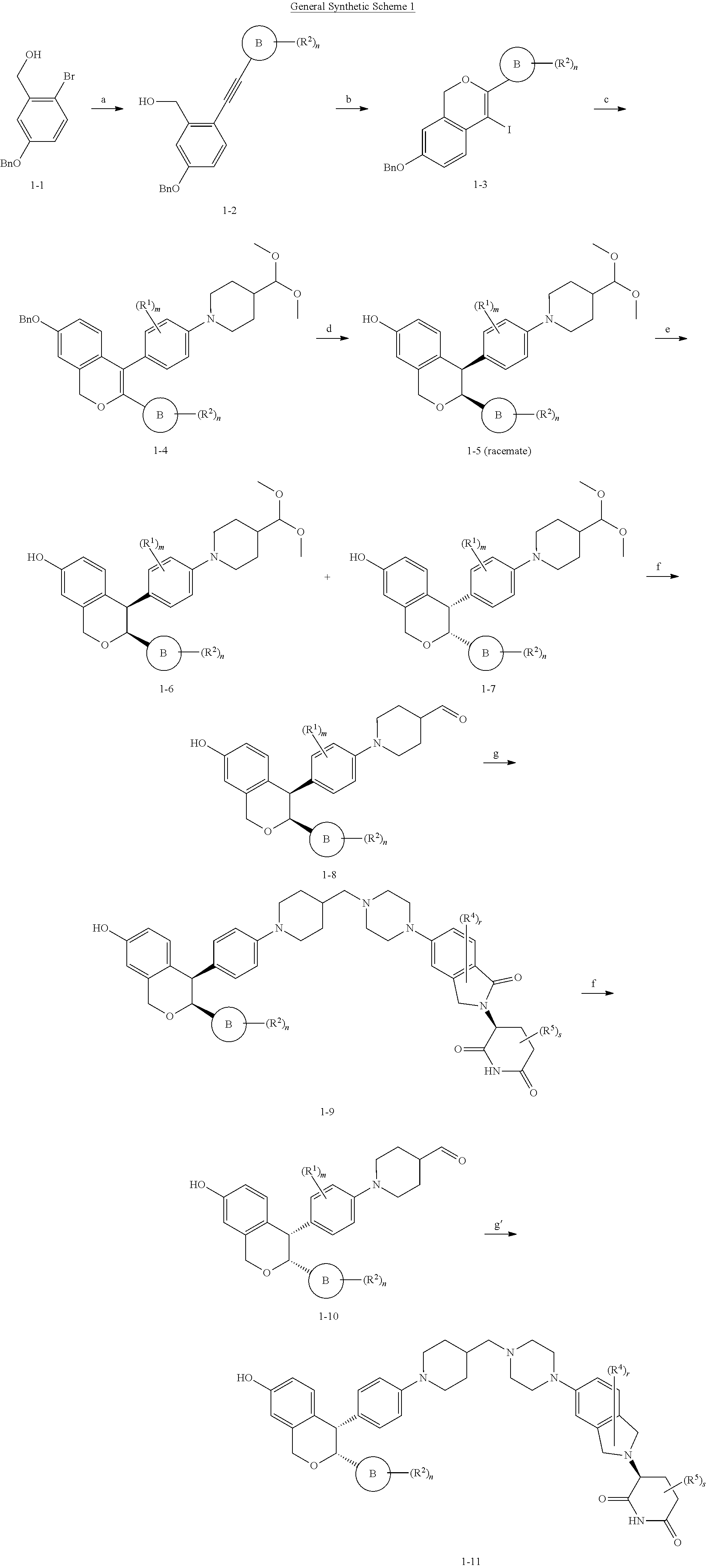

Reagents and conditions: (a) Pd(PPh₃)₂Cl₂, DMF, DIEA, CuI, ethynylbenzene, 120° C.; (b) I₂, NaHCO₃, CH₃CN, r.t. overnight; (c)

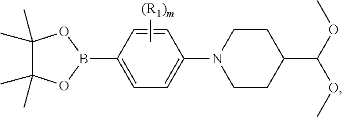

Pd(dppf)Cl₂, Na₂CO₃, dioxane:H₂O=4:1, 60° C., 16 h; (d) Pd/C, H₂, MeOH, r.t., 16 b, 15 Psi; (e) SFC (enantiomers processed separately after isolation); (f) or (f) 10% H₂SO₄, THF, 70° C., 3 h; (g) or (g′)

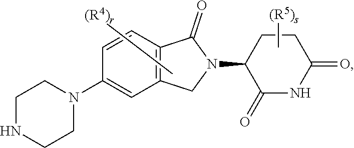

### NaBH₃CN, DCM:MeOH=1:1, 25° C., 12.5 h.

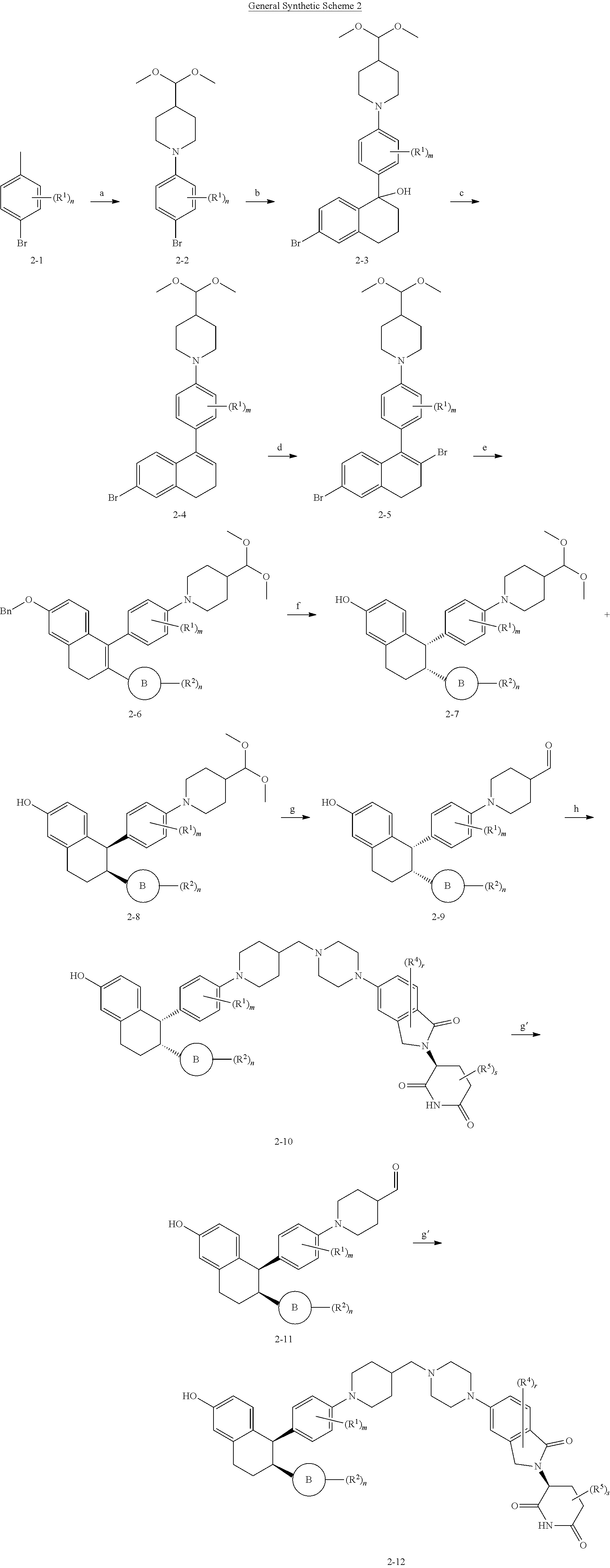

Reagents and conditions: (a)

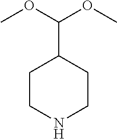

(1.10 eq), 0.40 eq L-proline, 2.00 eq K₂CO₃, 0.20 eq CuI, 10 V DMSO, 25˜80° C., 6 h; (b)

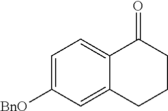 [SMILES: O=C1CCCc2cc(OCc3ccccc3)ccc21]

(1.05 eq), 1.40 eq n-BuLi, 10 V 2-MeTHF, −65° C., 3 h; (c) 0.02 eq TsOH, 10 V MeOH, 25˜65° C., 4 h; (d) 1.60 eq PyHBr₃, 2.00 eq DIPEA, 5 V DMF, 10˜25° C., 1 h; (e)

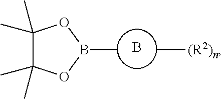

Pd-118, K₂CO₃, dioxane, 80° C., 12 h; (f) Pd/C, H₂, 25° C., 3 h; then SFC (enantiomers processed separately after isolation); (g) or (g′) 10% H₂SO₄, THF, 70° C., 3 h; (h) or (h′)

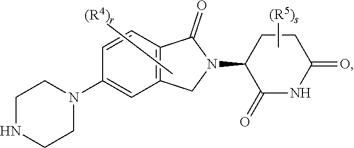

### NaBH₃CN, DCM:MeOH=1:1, 25° C., 12.5 h.

### Intermediate 1. Preparation of 7-(benzyloxy)-4-(4-bromophenyl)-4-hydroxyisochroman-3-one

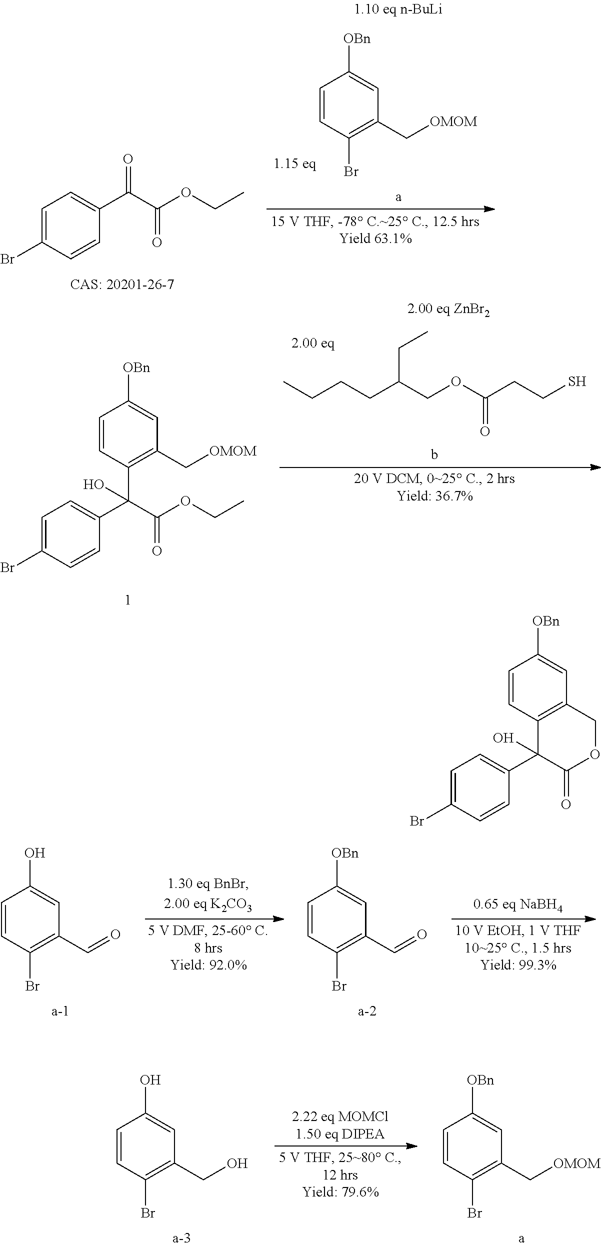

Step 1: To a solution of a-1 (300 g, 1.49 mol, 1.00 eq) in DMF (1.50 L) was added K₂CO₃ (412 g, 2.98 mol, 2.00 eq) and BnBr (331 g, 1.94 mol, 230 mL, 1.30 eq) at 25° C. The mixture was heated to 60° C. and stirred at 60° C. for 8 hrs. The combined mixture was diluted with ethyl acetate (1.50 L) and water (6.00 L), separated. The aqueous phase was extracted with ethyl acetate (1.50 L), then the combined organic layers were washed with brine (3.00 L), dried over Na₂SO₄. The mixture was triturated with Petroleum ether/Ethyl acetate=30/1 (3.20 L) at 25° C. for 3 hrs. The mixture was filtered and the filter cake was dried under reduce pressure to get a-2 (800 g, 2.75 mol, 92.0% yield) as an off-white solid.

¹H NMR: 400 MHz, CDCl₃

δ 10.31 (s, 1H), 7.52 (t, J=9.2 Hz, 2H), 7.45-7.30 (m, 5H), 7.20-7.05 (m, 1H), 5.10 (s, 2H)

Step 2: To a solution of a-2 (160 g, 549 mmol, 1.00 eq) in EtOH (1.60 L) and THF (160 mL) was added NaBH₄ (13.5 g, 357 mmol, 0.65 eq) in portions at 10˜20° C. over 30 mins. The mixture was warmed to 25° C. and stirred at 25° C. for 1 hr. The mixture was poured into aqueous NH₄Cl (4.50 L) slowly over 5 mins in parallel. Two batches reaction mixture were combined and extracted with ethyl acetate (3.00 L*2). The organic layers were washed with brine (5.00 L), dried over Na₂SO₄, filtered and concentrated to get a-3 (320 g, 1.09 mol, 99.3% yield) as a white solid.

¹H NMR: 400 MHZ, CDCl₃

δ 7.55-7.30 (m, 6H), 7.16 (d, J=2.8 Hz, 1H), 6.08 (t, J=3.2 Hz, 1H), 5.06 (s, 2H), 4.70 (s, 2H), 2.10 (s, 1H).

Step 3: To a solution of a-3 (210 g, 716 mmol, 1.00 eq) in THF (1.68 mL) was added DIPEA (138 g, 1.07 mol, 187 mL, 1.50 eq) and MOMCl (128 g, 1.59 mol, 120 mL, 2.22 eq) at 25° C. The mixture was heated to 80° C. and stirred at 80° C. for 12 hrs. Three batches of reaction were processed in parallel. The reaction was cooled to room temperature, diluted with ethyl acetate (500 mL), adjusted to pH=7 by saturated NaHCO₃ aqueous solution. The mixture was combined and separated, the aqueous phase was extracted with ethyl acetate (1.00 L*3). The organic layers was combined and washed with brine (2.00 L), dried over anhydrous Na₂SO₄, and concentrated under reduced pressure. The residue was purified by column chromatography (SiO₂, Petroleum ether/Ethyl acetate=1/0 to 0/1, R_{f}=0.40, Petroleum ether/Ethyl acetate=5/1) to get a (1.40 kg, 4.15 mol, 79.6% yield) as a white solid.

¹H NMR: 400 MHZ, CDCl₃

δ 7.50-7.35 (m, 6H), 7.17 (d, J=3.2 Hz, 1H), 6.80 (t, J=3.2 Hz, 1H), 5.07 (s, 2H), 4.77 (s, 2H), 4.64 (s, 2H), 3.44 (s, 3H)

Step 4: To a solution of a (151 g, 447 mmol, 1.15 eq) in THF (750 mL) was added n-BuLi (2.50 M, 171 mL, 1.10 eq) dropwise at −78° C. and stirred for 30 mins. Then CAS: 20201-26-7 (100 g, 389 mmol, 1.00 eq) in THF (750 mL) was added the mixture dropwise. The reaction was warmed to 25° C. and stirred for 12 hrs. The reaction was quenched by sat. NH₄Cl (1.00 L). The mixture was combined, extracted with ethyl acetate (1.00 L*3), dried with Na₂SO₄ and concentrated. The residue was purified by column chromatography (SiO₂, Petroleum ether/Ethyl acetate=1/0 to 0/1, R_{f}=0.50, Petroleum ether/Ethyl acetate=2/1) to give 1 (380 g, 737 mmol, 63.1% yield) as yellow gum.

¹H NMR: 400 MHZ, CDCl₃

δ 7.50-7.48 (m, 2H), 7.45-7.38 (m, 7H), 7.22 (d, J=2.8 Hz, 1H), 6.92-6.89 (m, 1H), 6.80-6.79 (m, 1H), 5.09 (s, 2H), 4.67-4.53 (m, 4H), 4.39-4.32 (m, 2H), 3.31 (s, 3H), 1.28 (t, J=7.2 Hz, 3H)

Step 5: To a solution of 1 (110 g, 213 mmol, 1.00 eq) in DCM (2.20 L) was added b (93.2 g, 426 mmol, 2.00 eq) and ZnBr₂ (96.1 g, 426 mmol, 21.3 mL, 2.00 eq) at 0° C., then warmed to 25° C. and stirred for 2 hrs. TLC (Petroleum ether/Ethyl acetate=3/1) showed that 1 (R_{f}=0.40) was consumed and one major spot (R_{f}=0.41) was formed. Three batches of reaction were processed in parallel. The mixture was concentrated and the residue was purified by column chromatography (SiO₂, Petroleum ether/Ethyl acetate=1/0 to 0/1, R_{f}=0.41, Petroleum ether/Ethyl acetate=3/1) to get 7-(benzyloxy)-4-(4-bromophenyl)-4-hydroxyisochroman-3-one (475 g, 1.12 mol, 36.7% yield, 90.7% purity) as a light yellow gum.

LCMS: RT=0.355 mins, product: m/z=407.0 (M−17)⁻

HPLC: product: RT=1.402 mins, 90.7% purity under 220 nm

¹H NMR: 400 MHZ, CDCl₃

δ 7.80 (d, J=8.8 Hz, 1H), 7.55-7.40 (m, 7H), 7.15 (d, J=8.4 Hz, 1H), 7.04 (d, J=8.8 Hz, 2H), 6.84 (s, 1H), 5.15-5.05 (m, 3H), 4.91 (d, J=13.6 Hz, 1H), 4.28 (s, 1H).

### Intermediate 2. Preparation of 2-(4-(benzyloxy)-2-(((tert-butyldimethylsilyl)oxy)methyl)phenyl)-2-(4-bromophenyl)-N-methoxy-N-methylacetamide

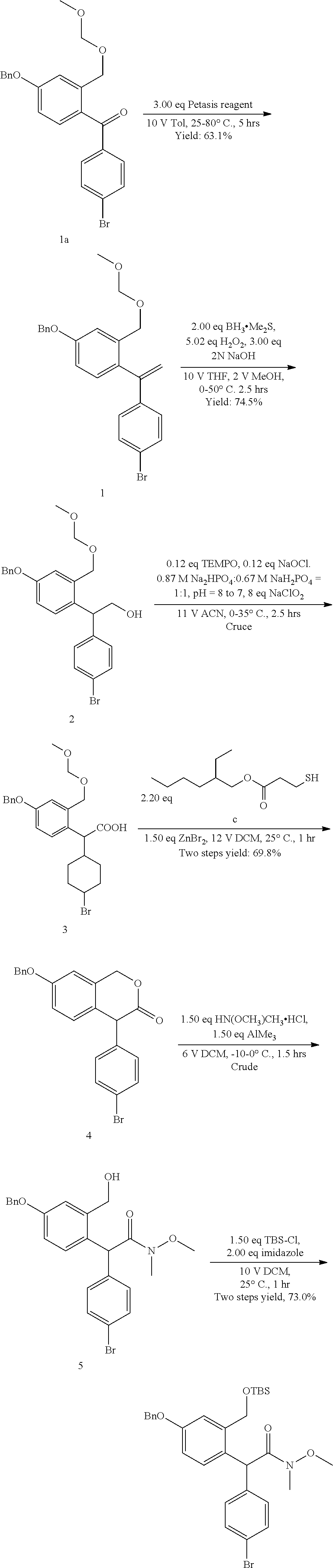

Step 1: To a solution of 1a (100 g, 226 mmol, 1.00 eq) in Tol. (800 mL) was added cyclopentane;dimethyltitanium (0.90 M, 755 mL, 3.00 eq) at 25° C. under N₂. The mixture was heated to 80° C. and stirred at 80° C. for 5 hrs. The crude product was purified by column chromatography (SiO₂, petroleum ether/ethyl acetate=20/1 to 15/1, petroleum ether/ethyl acetate=5/1, R_{f}=0.60). Compound 1 (442 g, 1.01 mol, 63.1% yield) was obtained as yellow oil and confirmed by ¹H NMR.

¹H NMR: 400 MHZ, CDCl₃

δ 7.46-7.42 (m, 2H), 7.41-7.40 (m, 4H), 7.40-7.39 (m, 1H), 7.17-7.11 (m, 4H), 6.94-6.91 (m, 1H), 5.75 (s, 1H), 5.23 (s, 1H), 5.11 (s, 2H), 4.55 (s, 2H), 4.33 (s, 2H), 3.28 (s, 3H).

Step 2: To a solution of compound 1 (116 g, 264 mmol, 1.00 eq) in THF (1.16 L) was added BH₃·Me₂S (10.0 M, 52.8 mL, 2.00 eq) at 25° C. under N₂. The mixture was heated to 50° C. and stirred at 50° C. for 0.5 hr. Then the mixture was cooled to 0° C. and MeOH (332 mL) was added to mixture slowly and NaOH (2 M, 396 mL, 3.00 eq) was added to mixture, then H₂O₂ (150 g, 1.33 mol, 127 mL, 30% purity, 5.02 eq) was added to mixture slowly. The mixture was warmed to 25° C. and stirred at 25° C. for 2 hrs. The mixture was poured into 10% Na₂S₂O₃ aqueous solution (1.16 L) slowly, stirred for 10 mins and separated, four batches of the organic phase was washed water (4.80 L), brine (4.80 L), dried over Na₂SO₄, filtered, and concentrated. The crude product was purified by column chromatography (SiO₂, Petroleum ether/Ethyl acetate=20/1 to 15/1, Petroleum ether/Ethyl acetate=2/1, R_{f}=0.30). Compound 2 (360 g, 753 mmol, 95.6% purity, 74.5% yield) was obtained as colorless oil and confirmed by LCMS and ¹H NMR.

LCMS: product: RT=0.999 min, m/z=481.2 (M+Na)+.

¹H NMR: 400 MHz, CDCl₃

δ 7.44-7.39 (m, 7H), 7.21-7.19 (m, 1H), 7.11-7.06 (m, 3H), 7.02-6.95 (m, 1H), 5.06 (s, 2H), 4.67-4.64 (m, 3H), 4.48-4.42 (m, 2H), 4.14-4.09 (m, 2H), 3.39 (s, 3H).

Step 3: To a solution of compound 2 (90.0 g, 197 mmol, 1.00 eq) in ACN (990 mL) was cooled to 0° C. and NaClO₂ (142 g, 1.57 mol, 8.00 eq) in H₂O (180 mL) was added to mixture. Then TEMPO (3.71 g, 23.6 mmol, 0.12 eq), Na₂HPO₄ (0.67 M, 294 mL, 1.00 eq) and NaH₂PO₄ (0.67 M, 294 mL, 1.00 eq) were added to mixture at 0° C. NaOCl (29.3 g, 23.6 mmol, 24.3 mL, 6% purity, 0.12 eq) in H₂O (180 mL) was added to mixture at 0° C. The mixture was warmed to 25° C. and stirred at 25° C. for 0.5 hr. Then the mixture was heated to 35° C. and stirred at 35° C. for 2 hrs. The mixture was poured into saturated Na₂SO₃ aqueous solution (1.50 L) slowly and stirred for 0.5 hr. Then extracted with ethyl acetate (800 mL*2), the organic phase was washed with Na₂SO₃ solution (4.00 L), brine (4.00 L), dried over Na₂SO₄, filtered, and concentrated. Compound 3 (363 g, crude) was obtained as yellow oil.

LCMS: product: RT=0.994 min, m/z=493.1 (M+Na)+.

Step 4: To a solution of compound 3 (121 g, 257 mmol, 1.00 eq) in DCM (1.45 L) was added ZnBr₂ (86.7 g, 385 mmol, 19.3 mL, 1.50 eq) and compound c (123 g, 565 mmol, 2.20 eq) at 25° C. The mixture was stirred at 25° C. for 1 hr. The crude product was purified by column chromatography (SiO₂, Petroleum ether/Ethyl acetate=20/1 to 15/1, Petroleum ether/Ethyl acetate=3/1, R_{f}=0.70). Compound 4 (220 g, 537 mmol, 69.8% yield over two steps) was obtained as off-white solid and confirmed by ¹H NMR.

¹H NMR: 400 MHZ, CDCl₃

δ 7.49-7.39 (m, 7H), 7.07-7.05 (m, 2H), 7.05-6.99 (m, 2H), 6.91 (s, 1H), 5.19 (s, 2H), 5.10 (s, 2H), 4.89 (s, 1H).

Step 5: To a solution of HN (OCH₃) CH₃·HCl (39.3 g, 403 mmol, 1.50 eq) in DCM (220 mL) was added AlMe₃ (2 M, 201 mL, 1.50 eq) at −10° C. and the mixture was stirred at 0° C. for 30 mins. Then compound 4 (110 g, 269 mmol, 1.00 eq) in DCM (440 mL) was added to reaction mixture at −10° C. and stirred at 0° C. for 1 hr. The mixture poured into saturated NH₄Cl aqueous solution (1.50 L) and extracted with DCM (500 mL*2), two batches of the organic phase was washed with brine (1.00 L), dried over Na₂SO₄, filtered, and concentrated. Compound 5 (240 g, crude) was obtained as off-white solid.

Step 6: To a solution of compound $ (120 g, 255 mmol, 1.00 eq) in DCM (1.20 L) was added TBSCl (57.7 g, 383 mmol, 47.0 mL, 1.50 eq) and imidazole (34.7 g, 510 mmol, 2.00 eq) at 25° C. The mixture was stirred at 25° C. for 1 hr. TLC (Petroleum ether/Ethyl acetate=2/1) showed compound 5 (R_{f}=0.10) was consumed and a main spot (R_{f}=0.60) was formed. The mixture was poured into H₂O (1.20 L) and separated, the aqueous phase was extracted with DCM (500 mL). Two batches of the organic phase was washed with brine (1.00 L), dried over Na₂SO₄, filtered, and concentrated. The crude product was purified by column chromatography (SiO₂, Petroleum ether/Ethyl acetate=20/1 to 15/1, Petroleum ether/Ethyl acetate=3/1, R_{f}=0.50). 2-(4-(benzyloxy)-2-(((tert-butyldimethylsilyl)oxy)methyl)phenyl)-2-(4-bromophenyl)-N-methoxy-N-methylacetamide (240 g, 401 mmol, 73.0% yield over two steps, 97.8% purity) was obtained as yellow oil and confirmed by HPLC and ¹H NMR.

LCMS: product: RT=1.284 mins, m/z=586.2 (M+H)+.

HPLC: product: RT=4.546 mins, 97.8% purity under 220 nm.

¹H NMR: 400 MHZ, CDCl₃

δ 7.44-7.37 (m, 7H), 7.20-7.18 (m, 1H), 7.09-7.07 (m, 3H), 6.88-6.87 (m, 1H), 5.63 (s, 1H), 5.06 (s, 2H), 4.70-4.57 (m, 2H), 3.39 (s, 3H), 3.22 (s, 3H), 0.91 (s, 9H), 0.05-0.03 (m, 6H).

### Intermediate 3. Preparation of 3-(1-methyl-6-(piperazin-1-yl)-1H-indazol-3-yl)piperidine-2,6-dione and 3-(1-methyl-7-(piperazin-1-yl)-1H-indazol-3-yl)piperidine-2,6-dione

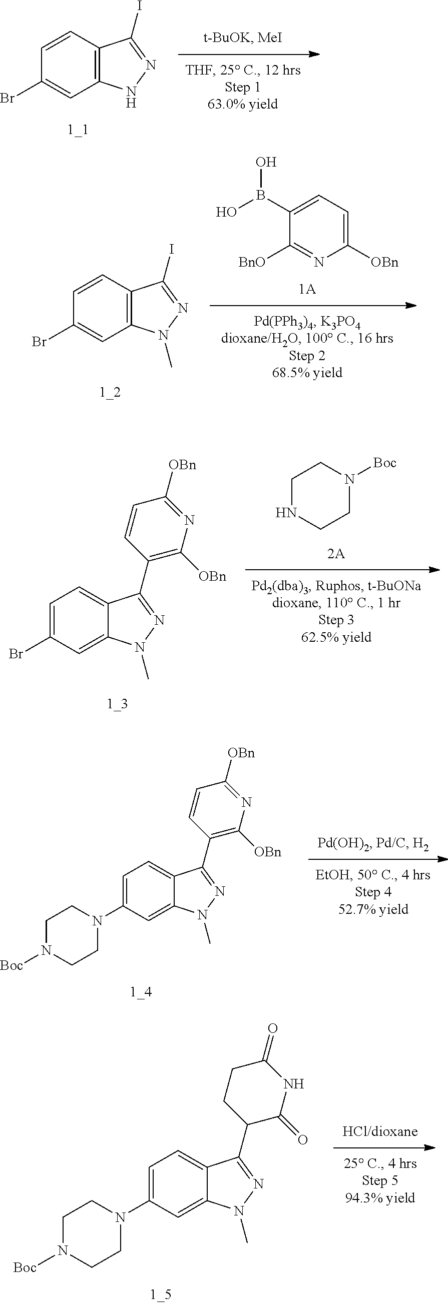

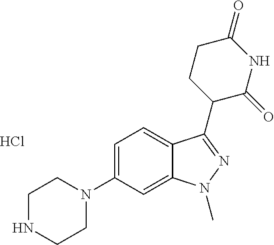 [SMILES: Cl ; Cn1nc(C2CCC(=O)NC2=O)c2ccc(N3CCNCC3)cc21]

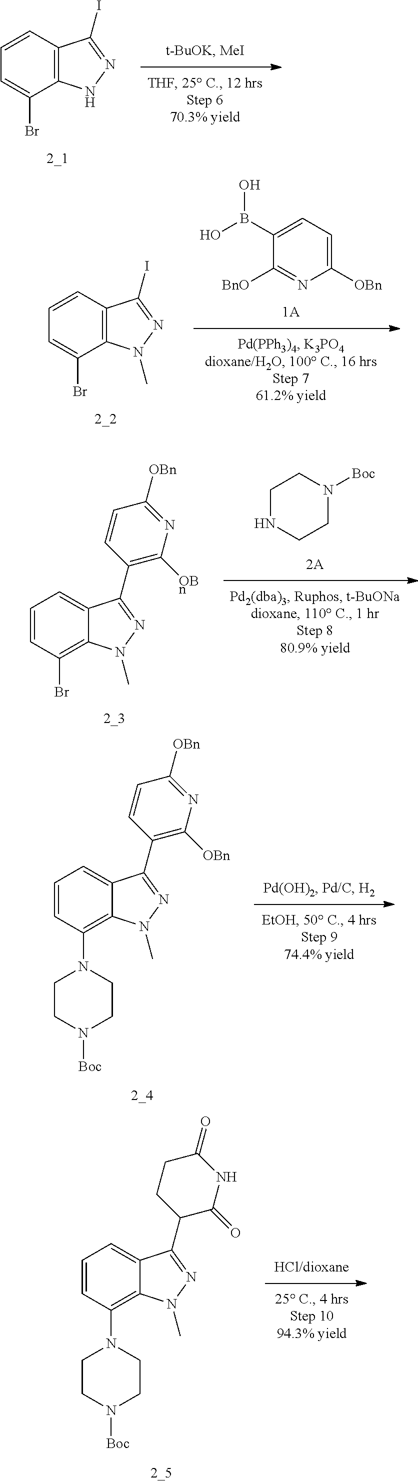

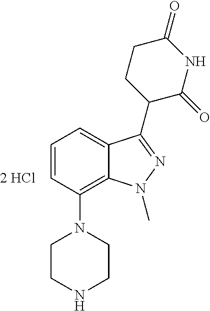 [SMILES: "2 HCl" ; Cn1nc(C2CCC(=O)NC2=O)c2cccc(N3CCNCC3)c21]

Step 1: To a solution of compound 1_1 (40.0 g, 124 mmol, 1.00 eq) in THE (400 mL) was added t-BuOK (41.7 g, 372 mmol, 3.00 eq). The mixture was stirred for 30 mins at 25° C. under N₂ atmosphere. And then Mel (26.4 g, 186 mmol, 1.50 eq) in THF (50.0 mL) was added dropwise. The resulting mixture was stirred at 0˜25° C. for 12 hrs. TLC (Petroleum ether:EtOAc=10:1, compound 1_1 R_{f}=0.40, compound 1_2 R_{f}=0.60) indicated compound 1_1 was consumed completely, and one new spot was detected. The resultant mixture was diluted with H₂O 500 mL and extracted with EtOAc 600 mL (300 mL*2), dried over anhydrous Na₂SO₄, filtered and concentrated under reduced pressure to give a residue. The residue was purified by column chromatography (SiO₂, Petroleum ether:EtOAc=40:1 to 10:1) to give compound 1_2 (26.3 g, 98.5% purity, 63.0% yield) was obtained as a yellow solid.

LCMS: product RT=0.463 min, 98.5% purity, m/z=337.0 [M+H]⁺

¹H NMR: (400 MHZ, DMSO-d₆)

δ 8.02 (d, J=0.63 Hz, 1H) 7.29-7.38 (m, 2H) 4.05 (s, 3H)

Step 2: To a solution of compound 1_2 (25.0 g, 74.1 mmol, 1.00 eq), compound 1A (29.7 g, 88.9 mmol, 1.20 eq), K₃PO₄ (49.0 g, 231 mmol, 3.00 eq) and Pd(PPh₃) 4 (8.50 g, 8.20 mmol, 0.10 eq) in dioxane (280 mL) and H₂O (30.0 mL) was degassed and purged with N₂ for 3 times, and then the mixture was stirred at 100° C. for 16 hrs under N₂ atmosphere. TLC (Petroleum ether:EtOAc=10:1, compound 1_2 R_{f}=0.60, compound 1_3 R_{f}=0.20) indicated compound 1_2 was consumed completely, and one new spot was detected. The resultant mixture was diluted with H₂O (200 mL) at 25° C. and extracted with EtOAc (300 mL*2). The combined organic layers were washed with brine 300 mL, dried over anhydrous Na₂SO₄, filtered and concentrated under reduced pressure to give a residue. The residue was purified by column chromatography (SiO₂, Petroleum ether:EtOAc=40:1 to 2:1) to give compound 1_3 (28.8 g, 57.5 mmol, 68.5% yield) as a yellow solid.

LCMS: product RT=0.678 min, 97.6% purity, m/z=502.2 [M+H]⁺

Step 3: A mixture of compound 2_3 (28.0 g, 55.9 mmol, 1.00 eq), compound 2A (15.6 g, 83.9 mmol, 1.50 eq), Pd(dba)₂ (5.13 g, 5.59 mmol, 0.10 eq), RuPhos (2.61 g, 5.59 mmol, 0.10 eq) and t-BuONa (16.1 g, 168 mmol, 3.00 eq) in dioxane (300 mL) was degassed and purged with N₂ for 3 times, and then the mixture was stirred at 110° C. for 1 hr under N₂ atmosphere. LCMS (product: RT=0.661 min) showed compound 1_3 was consumed completely and 56.7% of compound 1_4 was detected. The reaction was concentrated under reduced pressure to remove solvent. The residue was dissolved in H₂O 250 mL, washed with EtOAc 700 mL (350 mL*2). The combined organic layers were washed with brine 400 mL, dried over anhydrous Na₂SO₄, filtered and concentrated under reduced pressure to give a residue. The residue was purified by column chromatography (SiO₂, Petroleum ether:EtOAc=40:1 to 5:1) to give Compound 1_4 (21.2 g, 35.0 mmol, 62.5% yield) as a yellow solid.

LCMS: product RT=0.661 min, m/z=606.5 [M+H]⁺

Step 4: To a solution of compound 1_4 (21.0 g, 34.6 mmol, 1.00 eq) in EtOH (210 mL) was added Pd/C (20.0 g, 18.8 mmol, 10% purity) and Pd(OH)₂ (20.0 g, 28.5 mmol, 10% purity) under Ar₂ atmosphere. The suspension was degassed and purged with H₂ for 3 times. The mixture was stirred under H₂ (50 Psi) at 50° C. for 4 hrs. LCMS product: RT=0.444 min) showed compound 1_4 was consumed completely and 76.8% of compound 1_5 was detected. The reaction mixture was filtered and concentrated under reduced pressure to remove solvent. The crude product was triturated with EtOAc 15 mL and Petroleum ether 30 mL at 25° C. for 30 mins to give Compound 1_5 (7.78 g, 16.6 mmol, 52.7% yield) was obtained as a pink solid.

LCMS: product RT=0.444 min, m/z=428.4 [M+H]⁺

LCMS: product RT=0.414 min, m/z=428.4 [M+H]⁺

Step 5: To a solution of compound 1_5 (7.35 g, 17.2 mmol, 1.00 eq) in dioxane (75.0 mL) was added HCl/dioxane (4 M, 90.0 mL, 17.1 eq). The mixture was stirred at 25° C. for 4 hrs. LCMS (product: RT=0.267 min) showed compound 1_5 was consumed completely and 96.6% of 3-(1-methyl-7-(piperazin-1-yl)-1H-indazol-3-yl)piperidine-2,6-dione was detected. The reaction mixture was filtered and concentrated under reduced pressure to give 3-(1-methyl-6˜(piperazin-1-yl)-1H-indazol-3-yl)piperidine-2,6-dione (5.00 g, 15.3 mmol, 94.3% yield) as a white solid.

LCMS: product RT=0.267 min, m/z=328.2 [M+H]⁺

LCMS: product RT=0.773 min, m/z=328 [M+H]⁺

HPLC: product RT=1.130 mins, 92.7% purity

¹H NMR: (400 MHZ, DMSO-d₆)

δ 10.86 (s, 1H) 9.53 (s, 2H) 7.56 (d, J=8.88 Hz, 1H) 6.93-7.01 (m, 2H) 4.28 (dd, J=9.38, 5.13 Hz, 1H) 3.92 (s, 3H) 3.45-3.51 (m, 4H) 3.23 (s, 4H) 2.57-2.67 (m, 2H) 2.25-2.38 (m, 1H) 2.11-2.20 (m, 1H).

Step 6: To a solution of compound 2_1 (75.0 g, 232 mmol, 1.00 eq) in THF (400 mL) was added t-BuOK (78.2 g, 696 mmol, 3.00 eq). The mixture was stirred for 30 mins at 25° C. under N₂ atmosphere. And then Mel (49.4 g, 349 mmol, 1.50 eq) in THF (50.0 mL) was added dropwise. The resulting mixture was stirred at 25° C. for 12 hrs. TLC (Petroleum ether:EtOAc=10:1, compound 2_1 R_{f}=0.40, compound 2_2 R_{f}=0.60) indicated compound 2_1 was consumed completely, and one new spot was detected. The resultant mixture was diluted with H₂O (200 mL) at 0° C., and extracted with EtOAc 1.00 L (500 mL*2). The combined organic layers were washed with brine 300 mL, dried over Na₂SO₄, filtered and concentrated under reduced pressure to give a residue. The residue was purified by column chromatography (SiO₂, Petroleum ether:EtOAc=40:1 to 10:1) to give compound 2_2 (55.0 g, 163 mmol, 70.3% yield) as a yellow solid.

Step 7: To a solution of compound 2_2 (55.0 g, 163 mmol, 1.00 eq), compound 1A (71.7 g, 214 mmol, 1.20 eq), K₃PO₄ (104 g, 490 mmol, 3.00 eq) and Pd(PPh₃) 4 (18.9 g, 16.3 mmol, 0.10 eq) in dioxane (550 mL) and H₂O (55.0 mL) was degassed and purged with N₂ for 3 times, and then the mixture was stirred at 100° C. for 16 hrs under N₂ atmosphere. TLC (Petroleum ether:EtOAc=10:1, compound 2_2 R_{f}=0.60, compound 2_3 R_{f}=0.30) indicated compound 2_2 was consumed completely, and one new spot was detected. The resultant mixture was diluted with H₂O (200 mL) at 25° C. and extracted with EtOAc (300 mL*2). The combined organic layers were washed with brine 300 mL, dried over anhydrous Na₂SO₄, filtered and concentrated under reduced pressure to give a residue. The residue was purified by column chromatography (SiO₂, Petroleum ether:EtOAc=40:1 to 2:1) to give compound 2_3 (50.0 g, 99.9 mmol, 61.2% yield) as a yellow solid.

Step 8: A mixture of compound 2_3 (24.0 g, 48.0 mmol, 1.00 eq), compound 2A (17.9 g, 95.9 mmol, 2.00 eq), Pd(dba)₂ (2.76 g, 4.80 mmol, 0.10 eq), RuPhos (2.24 g, 4.80 mmol, 0.1 eq) and t-BuONa (13.8 g, 144 mmol, 3.00 eq) in dioxane (240 mL) was degassed and purged with N₂ for 3 times, and then the mixture was stirred at 110° C. for 1 hr under N₂ atmosphere. The reaction was concentrated under reduced pressure to remove solvent. The residue was dissolved in H₂O 250 mL, washed with EtOAc 700 mL (350 mL*2). The combined organic layers were washed with brine 400 mL, dried over anhydrous Na₂SO₄, filtered and concentrated under reduced pressure to give a residue. The residue was purified by column chromatography (SiO₂, Petroleum ether:EtOAc=40:1 to 5:1) to give Compound 2_4 (47.0 g, 77.6 mmol, 80.9% yield) as a yellow solid.

LCMS: RT=0.627 min, m/z=606.8 [M+H]⁺

Step 9: To a solution of compound 2_4 (20.0 g, 33.0 mmol, 1.00 eq) in EtOH (200 mL) was added Pd/C (20.0 g, 18.8 mmol, 10% purity) and Pd(OH)₂ (20.0 g, 28.5 mmol, 10% purity) under Ar₂ atmosphere. The suspension was degassed and purged with H₂ for 3 times. The mixture was stirred under H₂ (50 Psi) at 50° C. for 4 hrs. The reaction mixture was filtered and concentrated under reduced pressure to remove solvent. The crude product was triturated with EtOAc 15 mL and Petroleum ether 30 mL at 25° C. for 30 mins to give Compound 2_5 (11.5 g, 24.6 mmol, 74.4% yield) was obtained as a white solid.

LCMS: RT=0.455 min, m/z=428.5 [M+H]⁺

LCMS: RT=0.465 min, m/z=428.5 [M+H]⁺

Step 10: To a solution of compound 2_5 (9.00 g, 21.1 mmol, 1.00 eq) in dioxane (90 mL) was added HCl/dioxane (4 M, 90.0 mL, 17.1 eq). The mixture was stirred at 25° C. for 4 hrs. The reaction mixture was filtered and concentrated under reduced pressure to give 3-(1-methyl-7-(piperazin-1-yl)-1H-indazol-3-yl)piperidine-2,6-dione (6.50 g, 19.9 mmol, 94.3% yield) as a white solid.

LCMS: RT=0.280 min, m/z=328.1 [M+H]⁺

LCMS: RT=0.841 min, m/z=328.0 [M+H]⁺

HPLC: RT=0.757 min, 97.7% purity

¹H NMR: (400 MHZ, DMSO)

δ 10.89 (s, 1H), 9.61 (s, 1H), 9.52 (s, 1H), 9.71-9.39 (m, 1H), 9.19 (s, 1H), 7.47 (d, J=6.5 Hz, 1H), 7.12-7.01 (m, 2H), 4.36 (dd, J=5.0, 9.8 Hz, 1H), 4.25 (s, 3H), 3.42-3.09 (m, 8H), 2.73-2.58 (m, 2H), 2.39-2.28 (m, 1H), 2.25-2.09 (m, 1H).

### Intermediate 4. Preparation of 3-(3-methyl-2-oxo-5-(piperazin-1-yl)-2,3-dihydro-1H-benzo[d]imidazol-1-yl)piperidine-2,6-dione and 3-(3-methyl-2-oxo-4-(piperazin-1-yl)-2,3-dihydro-1H-benzo[d]imidazol-1-yl)piperidine-2,6-dione

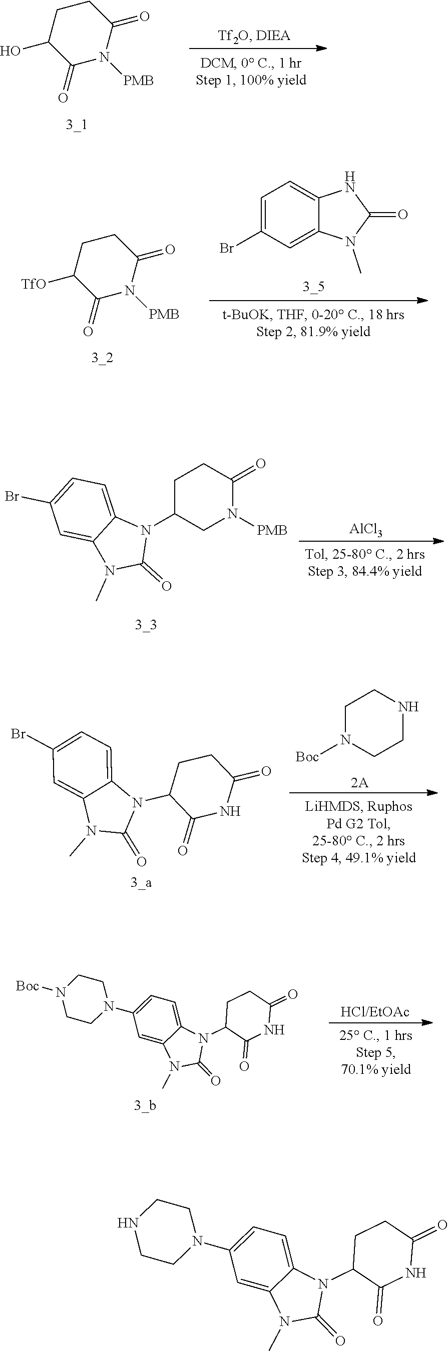

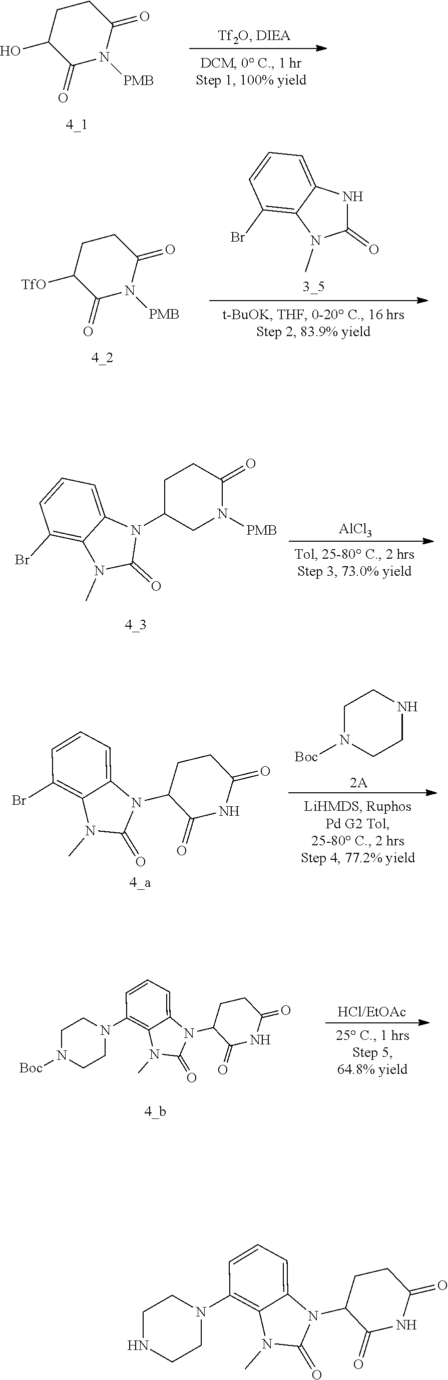

Step 1: To the solution of compound 3_1 (19.0 g, 76.2 mmol, 1.00 eq.) in DCM (20.0 mL) and MTBE (40.0 mL) was added Tf₂O (28.0 g, 99.1 mmol, 16.4 mL, 1.30 eq.) and DIEA (14.8 g, 114 mmol, 19.9 mL, 1.50 eq.) at 0° C., then the mixture was stirred at 25° C. for 0.5 hrs. HPLC detected compound 3_1 was consumed completed. The reaction material was diluted with H₂O (30.0 mL), and extracted with MTBE (40.0 mL), then the combined organic layers were used to next step.

HPLC: RT=1.778 min

Step 2: To the solution of compound 3_5 (9.50 g, 41.8 mmol, 1.00 eq.) in THF (95.0 mL) was added t-BuOK (7.51 g, 66.9 mmol, 1.60 eq.) at 0° C., then the mixture was stirred at 25° C. for 0.5 hr, then compound 3_2 (25.5 g, 66.9 mmol, 1.60 eq.) was added to the mixture at 0° C. and stirred at 25° C. for 12 hrs. HPLC detected compound 3_5 was consumed completed. The reaction mixture was filtered and concentrated under reduced pressure to give the compound 3_3 (16.0 g, 34.3 mmol, 81.9% yield, 98.1% purity) was obtained as a yellow solid, which was confirmed by HPLC.

HPLC: RT=1.761 min

HPLC: RT=1.067 min

Step 3: To the solution of compound 3_3 (15.0 g, 32.7 mmol, 1.00 eq.) in Tol. (150 mL) was added AlCl₃ (17.5 g, 131 mmol, 7.15 mL, 4.00 eq.) at 25° C., then the mixture was stirred at 80° C. for 2 hrs. TLC (PE:EA=3:1) showed the starting material (R_{f}=0.63) was consumed and one new spot (R_{f}=0.34) was formed. The reaction mixture was quenched by addition 1 M HCl (20.0 mL) at 5-10° C., then stirred at 25° C. for 0.5 hrs and concentrated under reduced pressure to give the compound 3_a (10.0 g, 27.6 mmol, 84.4% yield, 93.4% purity) as a gray solid, which was confirmed by HPLC.

HPLC: RT=1.767 min

Step 4: To the solution of compound 3_a (10.0 g, 29.6 mmol, 1.00 eq.) and compound 2A (8.26 g, 44.4 mmol, 1.50 eq.) in Tol. (50.0 mL) was added LiHMDS (1.00 M, 177 mL, 6.00 eq.) at 25° C. under N₂, then stirred for 0.5 hrs. [2-(2- aminophenyl)phenyl]-chloro-palladium;dicyclohexyl-[2-(2,6-diisopropoxyphenyl)phenyl]phosphane (2.30 g, 2.96 mmol, 0.10 eq.) and RuPhos (1.38 g, 2.96 mmol, 0.10 eq.) was added to the reaction at 25° C. under N₂. The mixture was stirred at 80° C. for 2 hrs. TLC (DCM:MeOH=20:1) showed the starting material (R_{f}=0.62) was consumed and one new spot (R_{f}=0.25) was formed. The reaction mixture was quenched by addition HCOOH (40.0 mL) at 0° C., and then diluted with H₂O (150 mL) and extracted with DCM (100 mL), the organic phase was separated and the water phase was extracted by DCM (100 mL), combined organic phase, washed by brine (100 mL), dried over Na₂SO₄, filtered and concentrated under reduced pressure to give the compound 3_b (9.00 g, 14.5 mmol, 49.1% yield, 71.5% purity) as a brown oil, which was confirmed by HPLC.

HPLC: RT=1.184 min.

Step 5: The solution of compound 3_b (9.00 g, 20.3 mmol, 1.00 eq.) in HCl/EtOAc (100 mL) was stirred at 25° C. for 1 hr. TLC (DCM:MeOH=20:1) showed compound 3_b (R_{f}=0.48) was consumed and one new spot was formed (Product R_{f}=0.00). The reaction mixture was filtered and concentrated under reduced pressure to give a crude product. The crude product was purified by Pre-HPLC to give the 3-(3-methyl-2-oxo-5-(piperazin-1-yl)-2,3-dihydro-1H-benzo[d]imidazol-1-yl)piperidine-2,6-dione (5.00 g, 14.2 mmol, 70.1% yield, 97.7% purity) as off-white solid, which was confirmed by ¹H NMR, LCMS and HPLC.

LCMS: RT=0.348 min, Ms+1=344.2

HPLC: RT=0.553 min

¹H NMR: 400 MHZ, DMSO-d₆

δ ppm 7.05-6.95 (m, 1H), 6.94-6.85 (m, 1H), 6.81-6.72 (m, 1H), 5.18 (br dd, J=5.6, 12.8 Hz, 1H), 3.62-3.53 (m, 2H), 3.36-3.28 (m, 3H), 3.26-3.20 (m, 4H), 3.14-3.02 (m, 2H), 2.87-2.75 (m, 1H), 2.72-2.54 (m, 2H), 2.09-1.99 (m, 1H).

Step 6: To the solution of compound 4_1 (19.0 g, 76.2 mmol, 1.00 eq.) in DCM (20.0 mL) and MTBE (40.0 mL) was added Tf₂O) (28.0 g, 99.1 mmol, 16.4 mL, 1.30 eq.) and DIEA (14.8 g, 114 mmol, 19.9 mL, 1.50 eq.) at 0° C., then the mixture was stirred at 25° C. for 1 hr. LCMS detected compound 4_1 was consumed completed. The reaction material was diluted with H₂O (100 mL), and extracted with MTBE (100 mL*2), then the combined organic layers were used to next step.

LCMS: RT=0.556 min

Step 7: To the solution of compound 4_5 (9.50 g, 41.8 mmol, 1.00 eq.) in THF (100 mL) was added t-BuOK (7.51 g, 66.9 mmol, 1.60 eq.) at 0° C., then the mixture was stirred at 25° C. for 0.5 hr, then compound 4_2 (25.5 g, 66.9 mmol, 1.60 eq.) was added to the mixture at 0° C. and stirred at 25° C. for 16 hrs. HPLC detected compound 4_5 was consumed completed. The reaction mixture was filtered and concentrated under reduced pressure to give the compound 4_3 (17.1 g, 35.1 mmol, 83.9% yield, 94.1% purity) was obtained as a gray solid, which was confirmed by HPLC.

HPLC: RT=1.807 min

HPLC: RT=1.067 min

Step 8: To the solution of compound 3_3 (17.0 g, 37.1 mmol, 1.00 eq.) in Tol. (340 mL) was added AlCl₃ (19.8 g, 148 mmol, 8.11 mL, 4.00 eq.) at 25° C., then the mixture was stirred at 80° C. for 2 hrs. TLC (PE:EA=3:1) showed the starting material (R_{f}=0.60) was consumed and one new spot (R_{f}=0.30) was formed. The reaction mixture was quenched by addition HCl (400 mL, 1 N) at 5-10° C., then stirred at 25° C. for 0.5 hr and concentrated under reduced pressure to give the compound 4_a (9.60 g, 27.1 mmol, 73.0% yield, 95.4% purity) as a gray solid, which was confirmed by HPLC.

HPLC: RT=1.069 min

Step 9: To the solution of compound 4_a (9.60 g, 28.4 mmol, 1.00 eq.) and compound 2A (9.52 g, 51.1 mmol, 1.80 eq.) in Tol. (100 mL) was added LiHMDS (1 M, 369 mL, 13.0 eq.) at 0° C. under N₂, then stirred at 0° C. for 30 mins. [2-(2-aminophenyl)phenyl]-chloro-palladium; dicyclohexyl-[2-(2,6-diisopropoxyphenyl)phenyl]phosphane (2.21 g, 2.84 mmol, 0.10 eq.) and RuPhos (1.32 g, 2.84 mmol, 0.1 eq.) was added to the reaction at 25° C. under N₂. The mixture was stirred at 80° C. for 2 hrs. TLC (DCM:MeOH=20:1) showed the starting material (R_{f}=0.70) was consumed and one new spot (R_{f}=0.30) was formed. The reaction mixture was adjust pH=5-6 by addition HCOOH (40.0 mL) at 0° C., and then diluted with H₂O (150 mL) and extracted with DCM (100 mL), the organic phase was separated and the water phase was extracted by DCM (100 mL), combined organic phase, washed by brine (100 mL), dried over Na₂SO₄, filtered and concentrated under reduced pressure to give the compound 4_b (22.1 g, 21.93 mmol, 77.2% yield, 44.6% purity) as a brown oil, which was confirmed by HPLC.

HPLC: RT=1.474 min.

Step 10: The solution of compound 4_b (22.1 g, 21.9 mmol, 1.00 eq.) in HCV/EtOAc (300 mL) was stirred at 25° C. for 1 hr. TLC (DCM:MeOH=20:1) showed compound 4_b (R_{f}=0.40) was consumed and one new spot was formed (Product R_{f}=0.00). The reaction mixture was filtered and concentrated under reduced pressure to give a crude product. The crude product was purified by reversed-phase HPLC (0.1% HCl condition) to give 3-(3-methyl-2-oxo-4-(piperazin-1-yl)-2,3-dihydro-1H-benzo[d]imidazol-1-yl)piperidine-2,6-dione (5.50 g, 14.2 mmol, 64.8% yield, 98.1% purity, HCl) as white solid, which was confirmed by ¹H NMR, LCMS and HPLC. (column: Phenomenex luna C18 150*40 mm*15 um; mobile phase: [water (HCl)-ACN]; gradient: 22%-52% B over 15 min).

LCMS: RT=0.326 min, Ms+1=344.1

HPLC: RT=0.670 min

¹H NMR: 400 MHZ, DMSO-d₆

δ ppm 7.07-7.03 (m, 1H), 6.98-6.92 (m, 2H), 5.26-5.22 (m, 1H), 3.59 (s, 3H), 3.30-3.14 (m, 6H), 3.10-3.06 (m, 2H), 2.82-2.79 (m, 1H), 2.66-2.50 (m, 2H), 2.02-1.97 (m, 1H).

### Intermediate 5. Preparation of (4-(benzyloxy)-2-((methoxymethoxy)methyl)phenyl) (4-bromophenyl) methanone

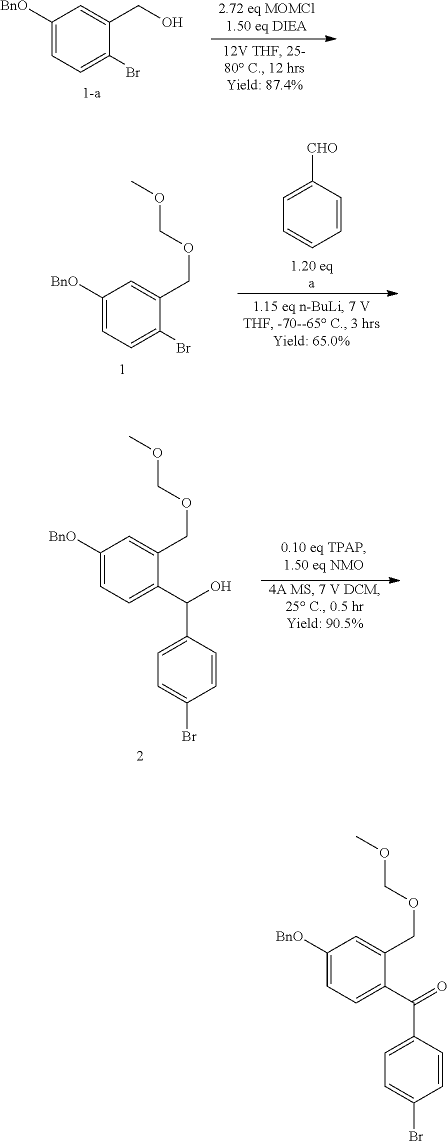 [SMILES: OCc1cc(OCc2ccccc2)ccc1Br ; COCOCc1cc(OCc2ccccc2)ccc1Br ; O=Cc1ccccc1 ; COCOCc1cc(OCc2ccccc2)ccc1C(O)c1ccc(Br)cc1 ; COCOCc1cc(OCc2ccccc2)ccc1C(=O)c1ccc(Br)cc1]

Step 1: To a solution of 1-a (150 g, 511 mmol, 1.00 eq) in THF (1.20 L) was added DIEA (99.2 g. 767 mmol, 133 mL, 1.50 eq) and MOMCl (112 g, 1.39 mol, 105 mL, 2.72 eq) at 25° C. The mixture heated to 80° C. and stirred at 80° C. for 12 hrs. TLC (petroleum ether:ethyl acetate=10:1) showed 1-a (Rt=0.10) was consumed and a new spot (R_{f}=0.60) was formed. Three batches were treated in parallel. The reaction was cooled to room temperature, diluted with 700 mL Ethyl acetate, adjusted to pH=7 by saturated NaHCO₃ aqueous solution. The mixture was separated, the aqueous phase was extracted with ethyl acetate (1.00 L)*3. The organic layers was combined and washed with brine (1.00 L), dried over anhydrous Na₂SO₄, and concentrated under reduced pressure. The crude product was purified by column chromatography (SiO₂, petroleum ether:ethyl acetate=30:1 to 20:1, petroleum ether:ethyl acetate=10:1, R_{f}=0.60). Compound 1 (502 g, 1.49 mol, 87.4% yield) was obtained as white solid.

¹H NMR: 400 MHz, CDCl₃

δ 7.60-7.45 (m, 6H), 7.17-7.16 (m, 1H), 6.80-6.78 (m, 1H), 5.07 (s, 2H), 4.76 (s, 2H), 4.63 (s, 2H), 3.34 (s, 3H).

Step 2: To a solution of compound 1 (187 g, 554 mmol, 1.00 eq) in THF (935 mL) was added n-BuLi (2.50 M, 255 mL, 1.15 eq) dropwise at −70-−60° C. under N₂. The mixture was stirred at −70° C. for 0.5 hr. Then compound a (123 g, 665 mmol, 1.20 eq) in THF (374 mL) was added to mixture slowly at −70° C. and stirred at −70-−60° C. for 2.5 hrs. TLC (Petroleum ether:Ethyl acetate=5:1) showed compound 1 (R_{f}=0.70) was consumed and a main spot (R_{f}=0.30) was formed. Two batches reaction were carried out in parallel. The mixture was poured into saturated NH₃Cl aqueous solution (3.00 L). The mixture was extracted with Ethyl acetate (1.50 L*3). The combined organic phase washed with brine (1.50 L), dried over Na₂SO₄, filtered, and concentrated. The crude product was purified by column chromatography (SiO₂, Petroleum ether:Ethyl acetate=10:1 to 5:1, petroleum ether:ethyl acetate=5:1, R_{f}=0.30). Compound 2 (296 g, 667 mmol, 65.0% yield) was obtained as colorless oil and confirmed by ¹H NMR.

¹H NMR: 400 MHZ, CDCl₃

δ 7.48-7.39 (m, 7H), 7.27-7.25 (m, 2H), 7.11 (d, J=8.0 Hz, 1H), 7.02 (d, J=2.8 Hz, 1H), 6.90-6.88 (m, 1H), 5.99 (d, J=2.4 Hz, 1H), 5.07 (s, 2H), 4.69-4.65 (m, 3H), 4.48 (d, J=12.0 Hz, 1H), 3.37 (s, 3H).

Step 3: To a solution of compound 2 (190 g, 428 mmol, 1.00 eq) in DCM (1330 mL) was added 4A MS (190 g), NMO (75.3 g, 642 mmol, 67.8 mL, 1.50 eq) and TPAP (15.0 g, 42.8 mmol, 0.100 eq) at 25° C. The mixture was stirred at 25° C. for 0.5 hr. TLC (petroleum ether:ethyl acetate=5:1) showed compound 2 (R_{f}=0.30) was consumed and a main new spot (R_{f}=0.60) was formed. The reaction was poured into 2.50 L of 10% Na₂S₂O₃ aqueous solution. This mixture was stirred for about 15 mins at 25° C., and then the entire mixture was filtered through a thin pad of celite to remove ruthenium solids and the sieves. The layers were then separated, the organic phase was washed with brine, dried over Na₂SO₄, filtered, and concentrated. The crude product was triturated with petroleum ether:ethyl acetate=10:1 (1.20 L) at 25° C. for 2 hrs. Filtrated and the filter cake was concentrated to give product 1 (175 g, 396 mmol, 90.5% yield) as a white solid.

Special LCMS: Product Rt=2.331 min (ELSD), (m/z)=441.0 (M+H)⁺

HPLC: product: RT=3.915 mins, 97.0% purity under 220 nm.

¹H NMR: 400 MHZ, CDCl₃

δ 7.65-7.60 (m, 4H), 7.45-7.35 (m, 6H), 7.31 (d, J=2.4 Hz, 1H), 6.91-6.89 (m, 1H), 5.16 (s, 2H), 4.80 (s, 2H), 4.63 (s, 2H), 3.33 (s, 3H).

### Intermediate 6. Preparation of 1-(4-bromo-2-methylphenyl)dihydropyrimidine-2,4 (1H,3H)-dione

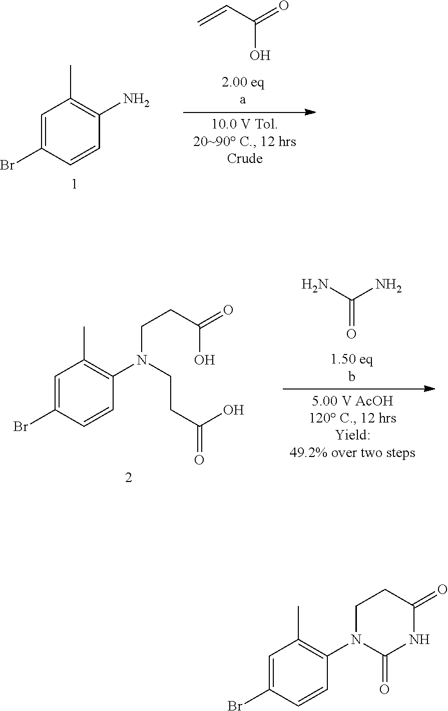

Step 1: To the stirred solution of compound 1 (20.0 g, 108 mmol, 1.00 eq) in Tol. (200 ml) was added compound a (15.5 g, 215 mmol, 14.7 mL, 2.00 eq) at 20° C. The mixture was heated to 90° C. and stirred at 90° C. for 12 hrs under N₂. LCMS showed that compound 1 was consumed and desired MS (RT=0.520 min) was detected. The reaction mixture was concentrated directly to give compound 2 (32.0 g, crude) as brown gum.

LCMS: product: RT=0.520 min, m/z=332.1 (M+H)⁺

Step 2: A mixture of compound 2 (32.0 g, 96.9 mmol, 1.00 eq) and compound b (8.73 g, 145 mmol, 7.82 mL, 1.50 eq) in AcOH (160 mL) was stirred at 120° C. for 12 hrs. LCMS showed compound 2 was consumed and desired MS (RT=0.482 min) was detected. The reaction mixture was cooled down to 20° C. and concentrated directly. The residue was adjusted to pH=8˜9 with Na₂CO₃ and extracted with ethyl acetate (500 mL*2). The combined organic phases were washed with brine (500 mL), dried over Na₂SO₄, concentrated under vacuum. The crude product was triturated with ethyl acetate (100 mL, 5.00 V) at 20° C. for 30 mins to give 1-(4-bromo-2-methylphenyl)dihydropyrimidine-2,4 (1H,3H)-dione (15.0 g, 51.3 mmol, 49.2% yield over two steps, 96.9% purity) as off-white solid.

LCMS: product: RT=0.482 min, m/z=283.1 (M+H)⁺

MS: m/z=283.1 (M+H)⁺

HPLC: product: RT=2.305 mins, 96.9% purity under 220 nm ¹H NMR: 400 MHZ, DMSO_d₆

δ 10.4 (s, 1H), 7.52 (d, J=1.6 Hz, 1H), 7.44 (dd, J=2.0 Hz, 2.0 Hz, 1H), 7.24 (d, J=8.8 Hz, 1H), 3.81-3.74 (m, 1H), 3.52-3.46 (m, 1H), 2.2.78-2.50 (m, 2H), 2.18 (s, 3H).

### Intermediate 7. Preparation of 1-(1-methyl-6-(piperazin-1-y)-1H-indazol-3-yl)dihydropyrimidine-2,4 (1H,3H)-dione

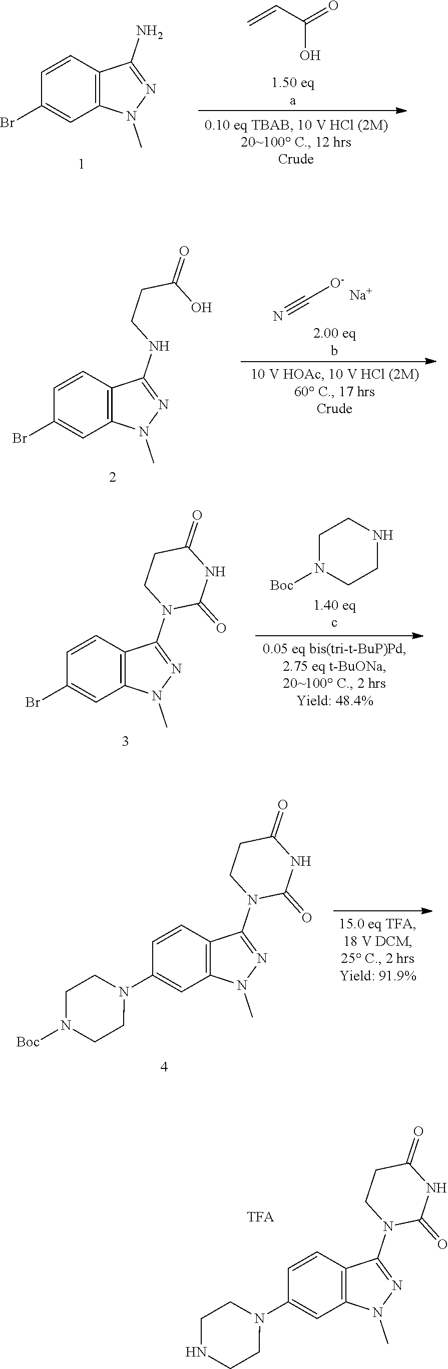

Step 1: To the stirred solution of compound 1 (50.0 g, 221 mmol, 1.00 eq) in HCl (2 M, 500 mL, 4.52 eq) (2 M aqueous solution) was added TBAB (7.13 g, 22.1 mmol, 0.10 eq) at 20° C. The reaction mixture was heated to 50° C. (internal temperature), and compound a (23.9 g, 332 mmol, 22.8 mL, 1.50 eq) was added dropwise. The reaction was then heated to 100° C. (external) for 12 hrs. After the reaction was complete, the reaction mixture was cooled to 20° C. and diluted with ice-cold water (2.00 L). It was neutralized to pH 6.5 to 7 with 2 M NaHCO₃ solution (2.00 L) with good stirring. The solid precipitation was filtered-off, washed with excess ice-cold water (500 mL), and dried under reduced pressure to give compound 2 (45.0 g, crude) as a yellow solid.

LCMS: product: Rt=0.468 min, m/z=299.7 (M+H)⁺

HPLC: product: Rt=1.591 mins, 81.4% purity under 220 nm

¹H NMR: (400 MHZ, DMSO-d₆)

δ 7.64-7.61 (m, 2H), 6.99-7.02 (m, 1H), 3.74 (s, 3H), 3.45-3.42 (m, 2H), 2.56-2.60 (m, 2H)

Step 2: To a solution of compound 2 (10.0 g, 33.5 mmol, 1.00 eq) in HOAc (100 mL) was added compound b (4.36 g, 67.1 mmol, 2.00 eq). The mixture was stirred at 60° C. for 14 hrs. Then HCl (2 M, 100 mL, 5.96 eq) (aqueous solution) was added, and the mixture was stirred at 60° C. for another 3 hrs. The reaction mixture was cooled to 20° C. A white solid was precipitated, which was filtered and washed with water (100 mL) to give compound 3 (11.7 g, crude) as a white solid.

LCMS: Rt=0.414 min, m/z=342.8 (M+NH₄)⁺

¹H NMR: (400 MHZ, DMSO-d₆) § 10.6 (s, 1H), 7.97 (d, J=1.0 Hz, 1H), 7.62 (d, J=8.8 Hz, 1H), 7.25 (dd, J=8.6, 1.6 Hz, 1H), 3.98 (s, 3H), 3.93 (t, J=6.6 Hz, 2H), 2.76 (t, J=6.6 Hz, 2H)

Step 3: Three batches of reaction were carried out in parallel.

To a stirred solution of compound 3 (4.00 g, 12.4 mmol, 1.00 eq) and compound c (3.23 g, 17.3 mmol, 1.40 eq) in dioxane (80.0 mL) was degassed with nitrogen for 3 mins. t-BuONa (3.27 g, 34.0 mmol, 2.75 eq) and bis(tri-t-BuP)Pd (316 mg, 619 μmol, 0.05 eq) was added to the reaction mixture at 20° C. and further degassed with nitrogen for 3 mins. The reaction vial was heated at 100° C. for 2 hrs under N₂. Three batches were combined to work up. The reaction mixture was concentrated under vacuum at 45° C. The residue was purified by silica gel chromatography (Petroleum ether/Ethyl acetate=1/1 to 0/1, Petroleum ether/Ethyl acetate=0/1, R_{f}=0.20). The fraction was concentrated under vacuum to give compound 4 (8.60 g, 18.0 mmol, 48.4% yield) as a yellow solid.

LCMS: Rt=0.479 min, m/z=429.2 (M+H)⁺

¹H NMR: (400 MHz, DMSO-d₆)

δ 10.5 (s, 1H), 7.48 (d, J=9.0 Hz, 1H), 6.97-6.85 (m, 2H), 3.93-3.86 (m, 5H), 3.53-3.44 (m, 4H), 3.22-3.14 (m, 4H), 2.73 (t, J=6.6 Hz, 2H), 1.43 (s, 9H)

Step 4: To a stirred solution of compound 4 (7.70 g. 18.0 mmol, 1.00 eq) in DCM (140 mL) was added TFA (30.7 g, 270 mmol, 20.0 mL, 15.0 eq) at 25° C. The mixture was stirred at 25° C. for 2 hrs. The reaction mixture was concentrated under vacuum at 45° C. The residue was purified by prep-HPLC (TFA condition: column: Phenomenex luna c18 250 mm*100 mm*10 um; mobile phase: [water (TFA)-ACN]; gradient: 0%-30% B over 20 mins). The eluent was concentrated in vacuum to remove acetonitrile and H₂O, the residual aqueous was lyophilized to give 1-(1-methyl-6-(piperazin-1-yl)-1H-indazol-3-yl)dihydropyrimidine-2,4 (1H,3H)-dione (7.32 g, 16.5 mmol, 91.9% yield, 99.8% purity, TFA) as an off-white solid.

LCMS: Rt=0.663 min, m/z=329.2 (M+H)⁺

HPLC: Rt=1.331 mins, 99.8% purity under 220 nm

¹H NMR: (400 MHZ, DMSO-d₆)

δ 10.5 (s, 1H), 8.80 (d, J=1.2 Hz, 2H), 7.66-7.44 (m, 1H), 7.05-6.85 (m, 2H), 3.93-3.87 (m, 5H), 3.48-3.39 (m, 4H), 3.28 (s, 4H), 2.74 (t, J=6.6 Hz, 2H)

¹⁹F NMR: (400 MHz, DMSO-d₆)

δ −74.06.

Synthesis of Isochromanes from Weinreb Amides

### General Scheme:

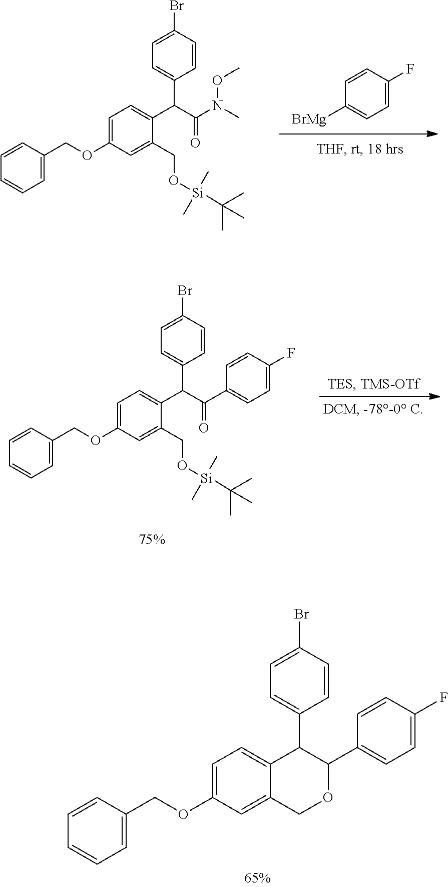

### Grignard Addition

### Intermediate 8. 2-(4-(benzyloxy)-2-(((tert-butyldimethylsilyl)oxy)methyl)phenyl)-2-(4-bromophenyl)-1-(4-fluorophenyl) ethan-1-one

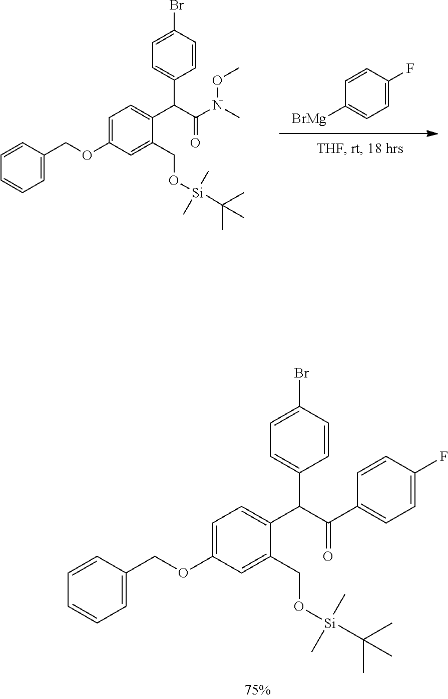

To a solution of 2-(4-(benzyloxy)-2-(((tert-butyldimethylsilyl)oxy)methyl)phenyl)-2-(4-bromophenyl)-N-methoxy-N-methylacetamide (500 mg, 1 Eq, 855 μmol) in THF (5 mL) was added 4-Fluorophenylmagnesium bromide, IM in diethyl ether (511 mg, 2.57 mL, 1 molar, 3 Eq, 2.57 mmol) at room temperature and the reaction mixture was allowed to stir at this temperature for 18 hours. The reaction mixture was quenched with ammonium chloride and extracted with ethyl acetate. The organic layer was separated, dried over sodium sulfate, filtered, and concentrated in vacuo. The residue was purified by silica gel chromatography, 24 g column, eluted with 0-50% ethyl acetate in heptane. The selected fractions were combined and concentrated in vacuo to give 2-(4-(benzyloxy)-2-(((tert-butyldimethylsilyl)oxy)methyl)phenyl)-2-(4-bromophenyl)-1-(4-fluorophenyl) ethan-1-one (400 mg, 646 μmol, 75.5%) as a colorless oil whose provisionally interpreted spectra are consistent with the assigned structure. ¹H NMR (500 MHz, CDCl₃) δ 8.09-8.01 (m, 2H), 7.46-7.32 (m, 7H), 7.07-6.94 (m, 6H), 6.81 (dd, J=8.5, 2.8 Hz, 1H), 6.31 (s, 1H), 5.02 (s, 2H), 4.70 (d, J=12.2 Hz, 1H), 4.45 (d, J=12.2 Hz, 1H), 0.92 (s, 9H), 0.08 (d, J=4.4 Hz, 6H).

### Intermediate 9. 2-(4-(benzyloxy)-2-(((tert-butyldimethylsilyl)oxy)methyl)phenyl)-1-(4-(benzyloxy)phenyl)-2-(4-bromophenyl) ethan-1-one

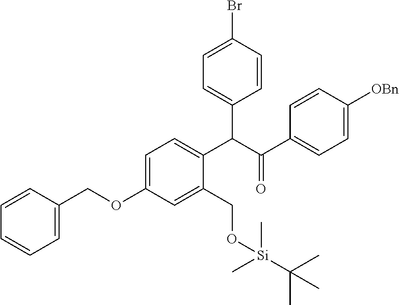 [SMILES: CC(C)(C)[Si](C)(C)OCc1cc(OCc2ccccc2)ccc1C(C(=O)c1ccc(OCc2ccccc2)cc1)c1ccc(Br)cc1]

¹H NMR (400 MHZ, CDCl₃) δ 8.06-7.91 (m, 2H), 7.58-7.30 (m, 12H), 7.12-6.97 (m, 4H), 6.97-6.85 (m, 2H), 6.81 (dd, J=8.5, 2.8 Hz, 1H), 6.27 (s, 1H), 5.09 (s, 2H), 5.02 (s, 2H), 4.69 (d, J=12.3 Hz, 1H), 4.46 (d, J=12.3 Hz, 1H), 0.91 (s, 9H), 0.07 (d, J=3.4 Hz, 6H).

### Intermediate 10. 2-(4-(benzyloxy)-2-(((tert-butyldimethylsilyl)oxy)methyl)phenyl)-2-(4-bromophenyl)-1-(5-fluoro-2-methylphenyl) ethan-1-one

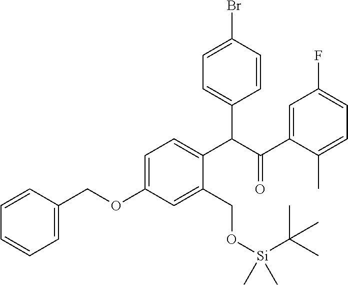 [SMILES: Cc1ccc(F)cc1C(=O)C(c1ccc(Br)cc1)c1ccc(OCc2ccccc2)cc1CO[Si](C)(C)C(C)(C)C]

¹H NMR (400 MHZ, CDCl₃) δ 7.49-7.28 (m, 8H), 7.17 (dd, J=8.5, 5.6 Hz, 1H), 7.08-6.98 (m, 5H), 6.84 (dd, J=8.5, 2.8 Hz, 1H), 6.18 (s, 1H), 5.04 (s, 2H), 4.63 (d, J=12.6 Hz, 1H), 4.49 (d, J=12.6 Hz, 1H), 2.45 (s, 3H), 0.89 (s, 9H), 0.04 (s, 6H).

### Intermediate 11. 2-(4-(benzyloxy)-2-(((tert-butyldimethylsilyl)oxy)methyl)phenyl)-2-(4-bromophenyl)-1-(3-fluoro-4-methylphenyl) ethan-1-one

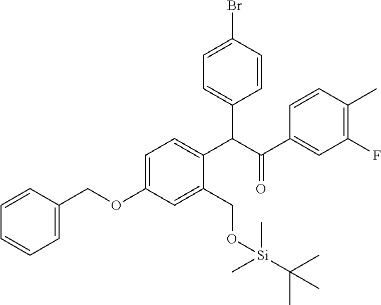 [SMILES: Cc1ccc(C(=O)C(c2ccc(Br)cc2)c2ccc(OCc3ccccc3)cc2CO[Si](C)(C)C(C)(C)C)cc1F]

¹H NMR (400 MHZ, CDCl₃) δ 7.73-7.61 (m, 2H), 7.47-7.28 (m, 7H), 7.17 (td, J=7.7, 0.9 Hz, 1H), 7.04-6.92 (m, 4H), 6.81 (dd, J=8.6, 2.8 Hz, 1H), 6.28 (s, 1H), 5.02 (s, 2H), 4.69 (d, J=12.3 Hz, 1H), 4.46 (d, J=12.3 Hz, 1H), 2.29 (d, J=1.9 Hz, 3H), 0.92 (s, 9H), 0.08 (d, J=2.4 Hz, 6H).

### Intermediate 12. 2-(4-(benzyloxy)-2-(((tert-butyldimethylsilyl)oxy)methyl)phenyl)-2-(4-bromophenyl)-1-(3-fluoro-5-methylphenyl) ethan-1-one

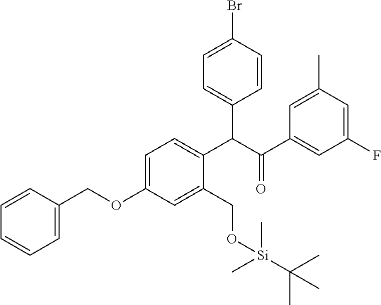 [SMILES: Cc1cc(F)cc(C(=O)C(c2ccc(Br)cc2)c2ccc(OCc3ccccc3)cc2CO[Si](C)(C)C(C)(C)C)c1]

¹H NMR (400 MHZ, CDCl₃) δ 7.56 (s, 1H), 7.51-7.28 (m, 8H), 7.01 (dt, J=6.1, 3.4 Hz, 4H), 6.94 (d, J=8.5 Hz, 1H), 6.82 (dd, J=8.5, 2.8 Hz, 1H), 6.26 (s, 1H), 5.03 (s, 2H), 4.68 (d, J=12.4 Hz, 1H), 4.48 (d, J=12.5 Hz, 1H), 2.35 (s, 3H), 0.91 (s, 9H), 0.06 (d, J=1.7 Hz, 6H).

### Intermediate 13. 2-(4-(benzyloxy)-2-(((tert-butyldimethylsilyl)oxy)methyl)phenyl)-2-(4-bromophenyl)-1-(4-fluoro-3-methylphenyl) ethan-1-one

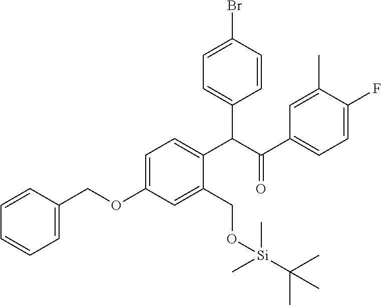 [SMILES: Cc1cc(C(=O)C(c2ccc(Br)cc2)c2ccc(OCc3ccccc3)cc2CO[Si](C)(C)C(C)(C)C)ccc1F]

¹H NMR (400 MHZ, CDCl₃) δ 7.89-7.80 (m, 2H), 7.46-7.31 (m, 7H), 7.04-6.90 (m, 5H), 6.81 (dd, J=8.5, 2.8 Hz, 1H), 6.28 (s, 1H), 5.02 (s, 2H), 4.68 (d, J=12.3 Hz, 1H), 4.46 (d, J=12.4 Hz, 1H), 2.26 (d, J=1.9 Hz, 3H), 0.91 (s, 9H), 0.06 (d, J=2.0 Hz, 6H).

### Intermediate 14. 2-(4-(benzyloxy)-2-(((tert-butyldimethylsilyl)oxy)methyl)phenyl)-2-(4-bromophenyl)-1-(4-fluoro-2-methylphenyl) ethan-1-one

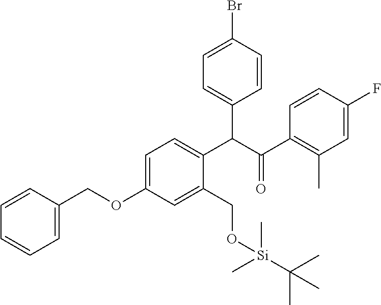 [SMILES: Cc1cc(F)ccc1C(=O)C(c1ccc(Br)cc1)c1ccc(OCc2ccccc2)cc1CO[Si](C)(C)C(C)(C)C]

¹H NMR (400 MHZ, CDCl₃) δ 7.84 (dd, J=8.7, 5.8 Hz, 1H), 7.48-7.28 (m, 7H), 7.06-6.97 (m, 4H), 6.92 (ddd, J=9.7, 2.7, 0.8 Hz, 1H), 6.88-6.76 (m, 2H), 6.22 (s, 1H), 5.03 (s, 2H), 4.63 (d, J=12.4 Hz, 1H), 4.46 (d, J=12.5 Hz, 1H), 2.54 (s, 3H), 0.90 (s, 9H), 0.05 (d, J=1.1 Hz, 6H).

### Intermediate 15. 2-(4-(benzyloxy)-2-(((tert-butyldimethylsilyl)oxy)methyl)phenyl)-2-(4-bromophenyl)-1-(3-fluoro-2-methylphenyl) ethan-1-one

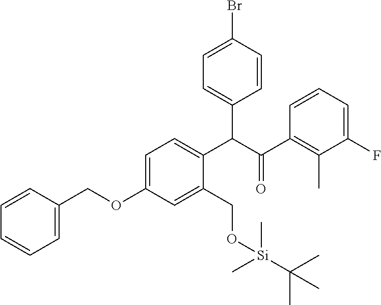 [SMILES: Cc1c(F)cccc1C(=O)C(c1ccc(Br)cc1)c1ccc(OCc2ccccc2)cc1CO[Si](C)(C)C(C)(C)C]

¹H NMR (400 MHZ, CDCl₃) δ 7.56-7.47 (m, 1H), 7.47-7.29 (m, 7H), 7.17-6.97 (m, 6H), 6.85 (dd, J=8.5, 2.8 Hz, 1H), 6.17 (s, 1H), 5.03 (s, 2H), 4.61 (d, J=12.6 Hz, 1H), 4.48 (d, J=12.6 Hz, 1H), 2.35 (d, J=2.4 Hz, 3H), 0.88 (s, 9H), 0.02 (s, 6H).

### Intramolecular Reductive Etherification of TBS-Ethers

### Intermediate 16. Cis-7-(benzyloxy)-4-(4-bromophenyl)-3-(4-fluorophenyl)isochromane

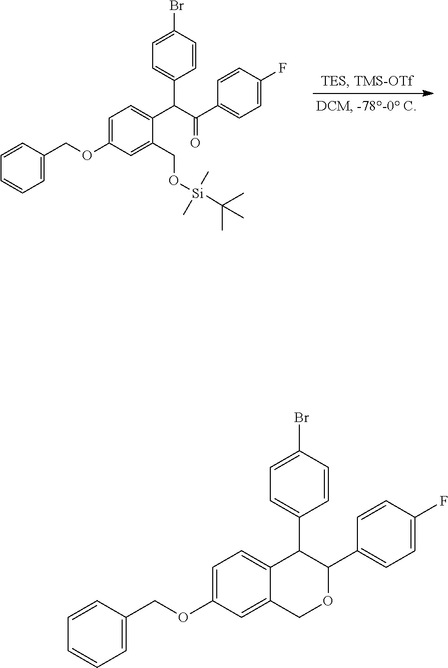 [SMILES: CC(C)(C)[Si](C)(C)OCc1cc(OCc2ccccc2)ccc1C(C(=O)c1ccc(F)cc1)c1ccc(Br)cc1 ; Fc1ccc(C2OCc3cc(OCc4ccccc4)ccc3C2c2ccc(Br)cc2)cc1]

To a solution of 2-(4-(benzyloxy)-2-(((tert-butyldimethylsilyl)oxy)methyl)phenyl)-2-(4-bromophenyl)-1-(4-fluorophenyl) ethan-1-one (398 mg, 1 Eq, 642 μmol) in DCM (3 mL) was added triethylsilane (112 mg, 154 μL, 1.5 Eq, 963 μmol), the resulting mixture was cooled at −78° C. and TMS-OTf (214 mg, 174 μL, 1.5 Eq, 963 μmol) was added, and the resulting mixture was allowed to stir for 30 min at this temperature a period during which the reaction was judged complete by LCMS. The reaction mixture was quenched with sodium bicarbonate, diluted with DCM and the resulting mixture stirred vigorously for 5 minutes. The organic layer was separated through a phase separator and concentrated in vacuo. The residue was purified by silica gel chromatography, 24 g column, eluted with Ea in Hpt, 0-40%. The selected fractions were combined and concentrated in vacuo to give (3S,4R)-7-(benzyloxy)-4-(4-bromophenyl)-3-(4-fluorophenyl)isochromane as a colorless oil whose provisionally interpreted spectra are consistent with the assigned structure. ¹H NMR (500 MHz, CDCl₃) δ 7.49-7.37 (m, 4H), 7.37-7.31 (m, 1H), 7.19-7.09 (m, 2H), 6.96-6.74 (m, 7H), 6.64-6.57 (m, 2H), 5.16 (d, J=15.3 Hz, 1H), 5.05 (dd, J=15.1, 3.3 Hz, 4H), 4.00 (d, J=3.3 Hz, 1H).

### Intermediate 17. 7-(benzyloxy)-4-(4-bromophenyl)-3-(3-fluoro-5-methylphenyl)isochromane

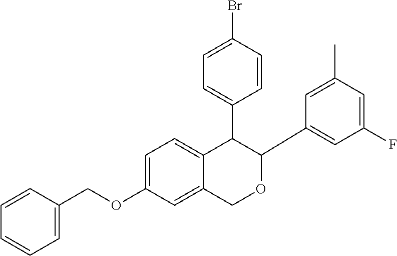 [SMILES: Cc1cc(F)cc(C2OCc3cc(OCc4ccccc4)ccc3C2c2ccc(Br)cc2)c1]

¹H NMR (500 MHZ, CDCl₃) δ 7.47-7.30 (m, 5H), 7.18-7.14 (m, 2H), 6.93 (d, J=8.5 Hz, 1H), 6.82 (dd, J=8.5, 2.6 Hz, 1H), 6.75 (d, J=2.6 Hz, 1H), 6.68-6.60 (m, 3H), 6.57 (td, J=1.5, 0.7 Hz, 1H), 6.51 (dd, J=9.7, 2.1 Hz, 1H), 5.16 (d, J=15.3 Hz, 1H), 5.10-4.98 (m, 4H), 4.02 (d, J=3.3 Hz, 1H), 2.22 (s, 3H).

### General Scheme

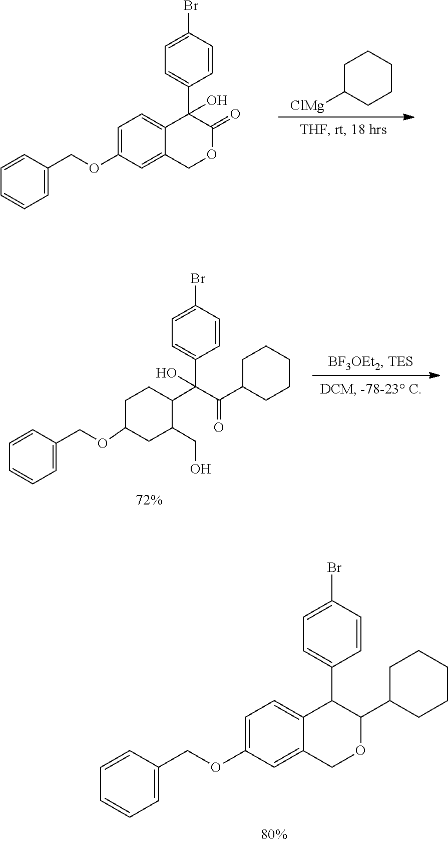

### Intermediate 18. 7-(benzyloxy)-4-(4-bromophenyl)-3-(4-fluoro-2-methylphenyl)isochromane

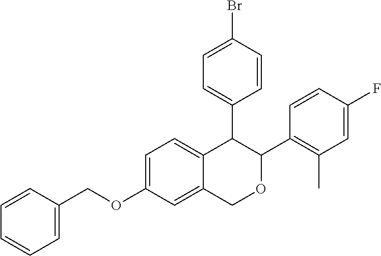 [SMILES: Cc1cc(F)ccc1C1OCc2cc(OCc3ccccc3)ccc2C1c1ccc(Br)cc1]

¹H NMR (500 MHZ, CDCl₃) δ 7.49-7.28 (m, 6H), 7.17-7.11 (m, 2H), 6.82 (td, J=9.6, 2.6 Hz, 2H), 6.77 (d, J=2.6 Hz, 1H), 6.62-6.49 (m, 4H), 5.20-5.02 (m, 5H), 4.03 (d, J=3.2 Hz, 1H), 2.38 (s, 3H).

### Intermediate 19. 7-(benzyloxy)-4-(4-bromophenyl)-3-(3-fluoro-2-methylphenyl)isochromane

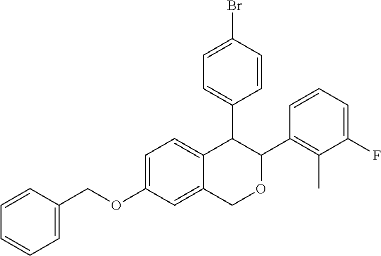 [SMILES: Cc1c(F)cccc1C1OCc2cc(OCc3ccccc3)ccc2C1c1ccc(Br)cc1]

¹H NMR (500 MHz, CDCl₃) δ 7.48-7.31 (m, 5H), 7.16-7.09 (m, 2H), 6.93 (d, J=8.5 Hz, 1H), 6.86-6.79 (m, 3H), 6.77 (d, J=2.6 Hz, 1H), 6.56-6.51 (m, 2H), 6.50-6.43 (m, 1H), 5.20-5.12 (m, 2H), 5.12-5.03 (m, 3H), 4.05 (d, J=3.2 Hz, 1H), 2.29 (d, J=2.1 Hz, 3H).

### Intermediate 20. 7-(benzyloxy)-4-(4-bromophenyl)-3-(5-fluoro-2-methylphenyl)isochromane

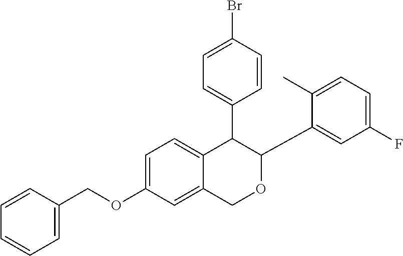 [SMILES: Cc1ccc(F)cc1C1OCc2cc(OCc3ccccc3)ccc2C1c1ccc(Br)cc1]

¹H NMR (400 MHZ, CDCl₃) δ 7.56-7.32 (m, 5H), 7.22-7.11 (m, 2H), 7.05 (dd, J=8.4, 5.7 Hz, 1H), 6.93 (d, J=8.5 Hz, 1H), 6.89-6.65 (m, 3H), 6.58-6.50 (m, 2H), 6.42 (dd, J=10.4, 2.9 Hz, 1H), 5.19 (s, 1H), 5.14-5.00 (m, 4H), 4.08 (d, J=3.2 Hz, 1H), 2.36 (s, 3H).

### Intermediate 21. 7-(benzyloxy)-3-(4-(benzyloxy)phenyl)-4-(4-bromophenyl)isochromane

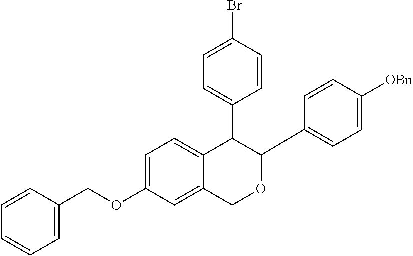 [SMILES: Brc1ccc(C2c3ccc(OCc4ccccc4)cc3COC2c2ccc(OCc3ccccc3)cc2)cc1]

¹H NMR (400 MHZ, CDCl₃) δ 7.51-7.28 (m, 11H), 7.22-6.55 (m, 10H), 5.23-4.87 (m, 6H), 4.53 (d, J=9.6 Hz, 1H), 4.16 (d, J=9.5 Hz, 1H).

### Intermediate 22. 7-(benzyloxy)-4-(4-bromophenyl)-3-(3-fluoro-4-methylphenyl)isochromane

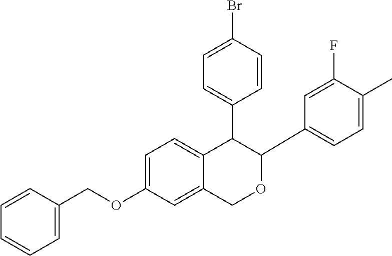 [SMILES: Cc1ccc(C2OCc3cc(OCc4ccccc4)ccc3C2c2ccc(Br)cc2)cc1F]

¹H NMR (400 MHZ, CDCl₃) δ 7.47-7.28 (m, 5H), 7.20-7.12 (m, 2H), 7.02-6.89 (m, 2H), 6.82 (dd, J=8.5, 2.7 Hz, 1H), 6.75 (d, J=2.6 Hz, 1H), 6.70-6.57 (m, 4H), 5.15 (d, J=15.3 Hz, 1H), 5.06 (d, J=2.0 Hz, 2H), 5.04-4.97 (m, 2H), 4.02 (d, J=3.3 Hz, 1H), 2.19 (d, J=1.9 Hz, 3H).

### Intermediate 23. 7-(benzyloxy)-4-(4-bromophenyl)-3-(4-fluoro-3-methylphenyl)isochromane

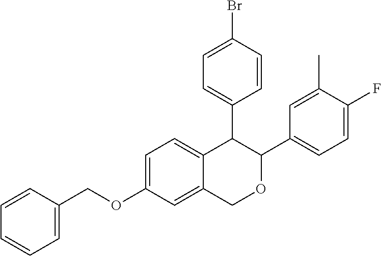 [SMILES: Cc1cc(C2OCc3cc(OCc4ccccc4)ccc3C2c2ccc(Br)cc2)ccc1F]

¹H NMR (500 MHz, CDCl₃) δ 7.46-7.31 (m, 5H), 7.24-7.13 (m, 2H), 6.92 (d, J=8.5 Hz, 1H), 6.84-6.71 (m, 5H), 6.63-6.58 (m, 2H), 5.15 (d, J=15.3 Hz, 1H), 5.11-4.96 (m, 4H), 3.98 (d, J=3.3 Hz, 1H), 2.13 (d, J=1.9 Hz, 3H).

### Intermediate 24. 7-(benzyloxy)-4-(4-bromophenyl)-3-(3-fluoro-2-methylphenyl)isochromane

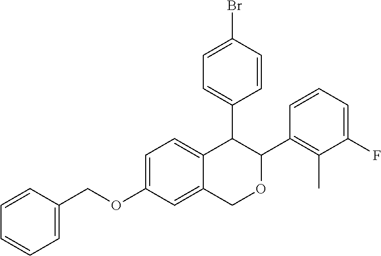 [SMILES: Cc1c(F)cccc1C1OCc2cc(OCc3ccccc3)ccc2C1c1ccc(Br)cc1]

¹H NMR (500 MHz, CDCl₃) δ 7.48-7.31 (m, 5H), 7.16-7.09 (m, 2H), 6.93 (d, J=8.5 Hz, 1H), 6.86-6.79 (m, 3H), 6.77 (d, J=2.6 Hz, 1H), 6.56-6.51 (m, 2H), 6.50-6.43 (m, 1H), 5.20-5.12 (m, 2H), 5.12-5.03 (m, 3H), 4.05 (d, J=3.2 Hz, 1H), 2.29 (d, J=2.1 Hz, 3H).

Synthesis of Isochromanes from α-Hydroxylactone
Grignard Addition into α-Hydroxylactone

### Intermediate 25. 2-(4-(benzyloxy)-2-(hydroxymethylphenyl)-2-(4-bromophenyl)-1-cyclohexyl-2-hydroxyethan-1-one

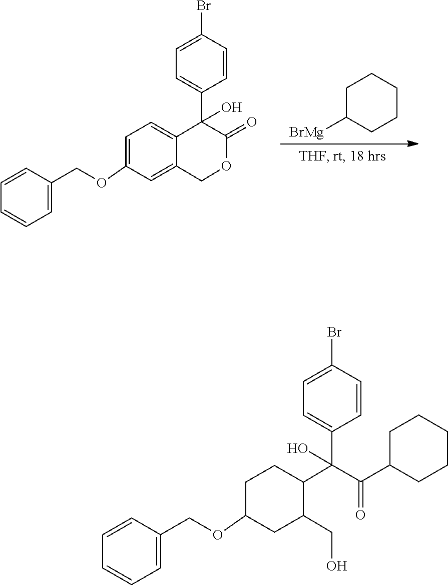

To a solution of 7-(benzyloxy)-4-(4-bromophenyl)-4-hydroxyisochroman-3-one (220 mg, 1 Eq, 517 μmol) in THF (2 mL) cooled at 0° C., on an ice bath, was added Cyclohexylmagnesium chloride solution in THE (296 mg, 2.07 mL, 1 molar, 4 Eq, 2.07 mmol) and the resulting mixture was allowed to warm up to room temperature and stirred 3 hours, at this time TLC, 30% Ea in Hptn, indicated about 50% conversion. More Cyclohexylmagnesium chloride solution in THE (296 mg, 2.07 mL, 1 molar, 4 Eq, 2.07 mmol), was added and the reaction allowed to continue for 12 hours. At this time the reaction was judged complete by TLC (7:3, Hptn/Ea). The mixture was cooled on ice, and quenched with ammonium chloride, and extracted with Ea. The organic layer was dried over sodium sulfate, filtered, and concentrated in vacuo. The residue was purified by silica gel chromatography, 12 g column, eluted with 0-30% ethyl acetate in heptane. The selected fractions were combined and concentrated in vacuo to give 2-(4-(benzyloxy)-2-(hydroxymethyl)phenyl)-2-(4-bromophenyl)-1-cyclohexyl-2-hydroxyethan-1-one (192 mg, 377 μmol, 72.9%) as a colorless oil whose provisionally interpreted spectra are consistent with the assigned structure. ¹H NMR (500 MHZ, CDCl₃) δ 7.46-7.35 (m, 8H), 7.34-7.30 (m, 1H), 7.23 (d, J=8.7 Hz, 1H), 6.81 (dd, J=8.7, 2.6 Hz, 1H), 6.60 (d, J=2.6 Hz, 1H), 5.08 (d, J=15.3 Hz, 1H), 5.01 (d, J=3.1 Hz, 2H), 4.79 (d, J=15.4 Hz, 1H), 3.10 (s, 1H), 2.78 (s, 1H), 2.22 (d, J=12.8 Hz, 1H), 1.91 (d, J=12.7 Hz, 1H), 1.71 (d, J=7.8 Hz, 1H), 1.64 (d, J=12.8 Hz, 1H), 1.57 (d, J=7.8 Hz, 1H), 1.32 (tt, J=12.0, 3.0 Hz, 1H), 1.10-0.85 (m, 5H).

### Intermediate 26. 2-(4˜(benzyloxy)-2-(hydroxymethyl)phenyl)-2-(4-bromophenyl)-2-hydroxy-1-(2-isopropylphenyl) ethan-1-one

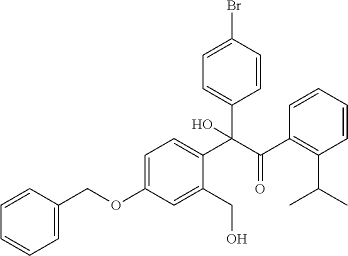 [SMILES: CC(C)c1ccccc1C(=O)C(O)(c1ccc(Br)cc1)c1ccc(OCc2ccccc2)cc1CO]

¹H NMR (400 MHZ, CDCl₃) δ 7.48-7.32 (m, 9H), 7.14-7.03 (m, 4H), 6.91 (dd, J=8.8, 3.0 Hz, 1H), 6.74-6.70 (m, 2H), 5.26 (d, J=15.4 Hz, 1H), 5.08 (s, 2H), 5.00 (d, J=15.4 Hz, 1H), 3.32 (s, 1H), 3.23 (s, 1H), 3.13 (s, 1H), 1.13 (d, J=6.7 Hz, 3H), 0.65 (d, J=6.9 Hz, 3H).

### Intramolecular Reductive Etherification & Dehydroxylation

### Intermediate 27. 7-(benzyloxy)-4-(4-bromophenyl)-3-cyclohexylisochromane

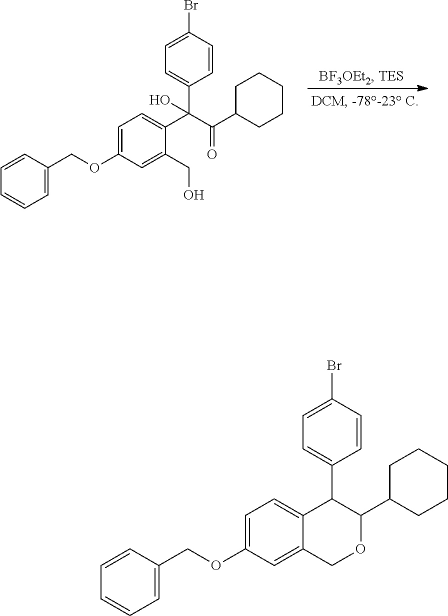 [SMILES: O=C(C1CCCCC1)C(O)(c1ccc(Br)cc1)c1ccc(OCc2ccccc2)cc1CO ; Brc1ccc(C2c3ccc(OCc4ccccc4)cc3COC2C2CCCCC2)cc1]

A mixture of 2-(4-(benzyloxy)-2-(hydroxymethyl)phenyl)-2-(4-bromophenyl)-1-cyclohexyl-2-hydroxyethan-1-one (192 mg, 1 Eq, 377 μmol) and triethylsilane (219 mg, 301 μL, 5 Eq, 1.88 mmol) in DCM (2 mL) was cooled on ice and triethylsilane (219 mg, 301 μL, 5 Eq, 1.88 mmol) was added and the resulting mixture allowed to warm up to room temperature and stirred for 18 hours, a period during which the starting material was fully consumed to form a new slightly more polar compound as judged by TLC (7:3, Hptn/Ea). The reaction mixture was quenched with sodium bicarbonate, and extracted with DCM. The organic layer was separated through a phase separator and concentrated in vacuo.

The residue was purified by silica gel chromatography, 12 g column, eluted with 0-30% ethyl acetate in heptane to give 7-(benzyloxy)-4-(4-bromophenyl)-3-cyclohexylisochromane (145 mg, 304 μmol, 80.6%) as a white solid whose provisionally interpreted spectra are consistent with the assigned structure. 1H NMR (500 MHZ, CDCl₃) δ 7.45-7.29 (m, 7H), 7.20-7.15 (m, 2H), 6.89 (d, J=8.5 Hz, 1H), 6.76-6.69 (m, 1H), 6.64 (dd, J=13.0, 3.1 Hz, 1H), 5.10-4.95 (m, 3H), 4.87-4.78 (m, 1H), 3.86 (d, J=2.9 Hz, 1H), 3.51-3.41 (m, 1H), 2.12-2.00 (m, 1H), 1.82 (d, J=12.5 Hz, 1H), 1.72-1.57 (m, 3H), 1.18-1.00 (m, 4H), 0.93 (dt, J=24.6, 12.8 Hz, 2H).

### Intermediate 28. 7-(benzyloxy)-4-(4-bromophenyl)-3-(2-isopropylphenyl)isochromane

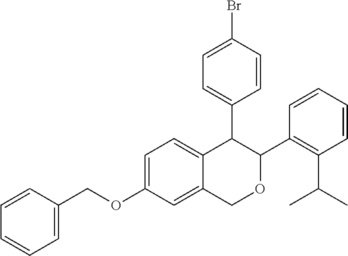 [SMILES: CC(C)c1ccccc1C1OCc2cc(OCc3ccccc3)ccc2C1c1ccc(Br)cc1]

¹H NMR (500 MHZ, CDCl₃) δ 7.48-7.33 (m, 6H), 7.13 (dq, J=7.9, 2.0 Hz, 3H), 6.94 (d, J=8.5 Hz, 1H), 6.85-6.81 (m, 2H), 6.78 (d, J=2.7 Hz, 1H), 6.59 (dd, J=7.9, 1.4 Hz, 1H), 6.55-6.50 (m, 2H), 5.33-5.25 (m, 1H), 5.18 (d, J=15.3 Hz, 1H), 5.11-5.04 (m, 3H), 4.01 (d, J=3.1 Hz, 1H), 3.18 (p, J=6.9 Hz, 1H), 1.40 (d, J=6.8 Hz, 3H), 1.22 (d, J=6.8 Hz, 3H).

### Intermediate 29. 7-(benzyloxy)-4-(4-bromophenyl)-3-(4-fluoro-3-methylphenyl)isochromane

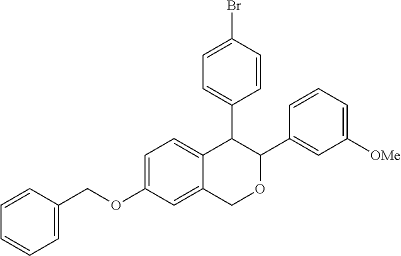 [SMILES: COc1cccc(C2OCc3cc(OCc4ccccc4)ccc3C2c2ccc(Br)cc2)c1]

¹H NMR (500 MHz, CDCl₃) δ 7.48-7.28 (m, 9H), 7.21-7.06 (m, 6H), 6.97-6.58 (m, 12H), 6.49-6.39 (m, 2H), 5.20-5.11 (m, 2H), 5.08-5.01 (m, 5H), 4.02 (d, J=3.3 Hz, 1H), 3.60 (s, 3H).

### Buchwald Amination

### Intermediate 30. 1-(4-(7-(benzyloxy)-3-(4-fluorophenyl)isochroman-4-yl)phenyl)-4-(dimethoxymethyl)piperidine

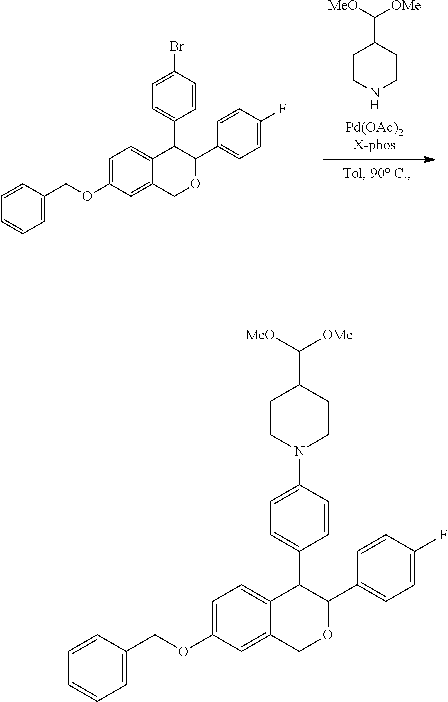 [SMILES: Fc1ccc(C2OCc3cc(OCc4ccccc4)ccc3C2c2ccc(Br)cc2)cc1 ; COC(OC)C1CCNCC1 ; COC(OC)C1CCN(c2ccc(C3c4ccc(OCc5ccccc5)cc4COC3c3ccc(F)cc3)cc2)CC1]

A mixture of (3S,4R)-7-(benzyloxy)-4-(4-bromophenyl)-3-(4-fluorophenyl)isochromane (277 mg, 1 Eq, 566 μmol), x-phos (54.0 mg, 0.2 Eq, 113 μmol), PdOAc2 (19.1 mg, 0.15 Eq, 84.9 μmol), 4-(dimethoxymethyl)piperidine (135 mg, 1.5 Eq, 849 μmol), and NaOtBu (81.6 mg, 1.5 Eq, 849 μmol) in Toluene (3 mL) was heated to 90° C. for 16 hours, a period during which the desired product was observed by LCMS. The reaction mixture was allowed to cool to room temperature, diluted with ethyl acetate and filtered through a short pad of celite. The residue was purified by silica gel chromatography, 24 g column, and eluted with 0-50% Ea, Hptn. The selected fractions were combined and concentrated in vacuo to give 1-(4-(7-(benzyloxy)-3-(4-fluorophenyl)isochroman-4-yl)phenyl)-4-(dimethoxymethyl)piperidine (200 mg, 352 μmol, 62.2%) as a white solid whose provisionally interpreted spectra are consistent with the assigned structure. ¹H NMR (500 MHz, CDCl₃) δ 7.48-7.29 (m, 5H), 6.99-6.88 (m, 3H), 6.83 (ddt, J=11.2, 8.2, 2.7 Hz, 3H), 6.74 (d, J=2.6 Hz, 1H), 6.60 (q, J=8.9 Hz, 4H), 5.16 (d, J=15.2 Hz, 1H), 5.10-5.02 (m, 3H), 4.99 (d, J=3.2 Hz, 1H), 4.05 (d, J=7.3 Hz, 1H), 3.95 (d, J=3.2 Hz, 1H), 3.59-3.54 (m, 2H), 3.35 (s, 6H), 2.54 (ddd, J=12.6, 9.8, 5.0 Hz, 2H), 1.79 (d, J=13.1 Hz, 2H), 1.70 (dtt, J=11.6, 7.6, 3.7 Hz, 1H), 1.41 (ddt, J=16.5, 12.2, 6.0 Hz, 2H).

### Intermediate 31. 1-(4-(7-(benzyloxy)-3-cyclohexylisochroman-4-yl)phenyl)-4-(dimethoxymethyl)piperidine

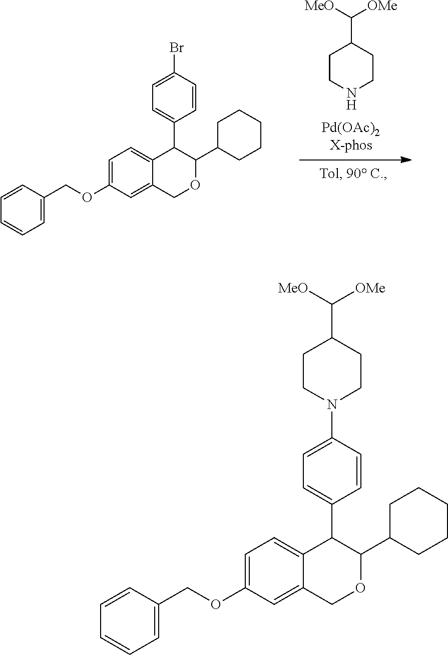 [SMILES: Brc1ccc(C2c3ccc(OCc4ccccc4)cc3COC2C2CCCCC2)cc1 ; COC(OC)C1CCNCC1 ; COC(OC)C1CCN(c2ccc(C3c4ccc(OCc5ccccc5)cc4COC3C3CCCCC3)cc2)CC1]

A mixture of 7-(benzyloxy)-4-(4-bromophenyl)-3-cyclohexylisochromane (145 mg, 1 Eq, 304 μmol), x-phos (29.0 mg, 0.2 Eq, 60.7 μmol), PdOAc2 (10.2 mg, 0.15 Eq, 45.6 μmol), 4-(dimethoxymethyl)piperidine (72.5 mg, 1.5 Eq, 456 μmol), and NaOtBu (43.8 mg, 1.5 Eq, 456 μmol) in Toluene (3 mL) was heated to 90° C. for 16 hours, a period during which the desired product was observed by LCMS. The reaction mixture was allowed to cool to room temperature, quenched with water and extracted with ethyl acetate. The organic layer was dried over sodium sulfate, filtered through a short pad of celite and concentrated in vacuo. The residue was purified by silica gel chromatography, 24 g column, dry-loading on celite, and eluted with 0-50% Ea, Hptn. The selected fractions were combined and concentrated in vacuo to give 1-(4-(7-(benzyloxy)-3-cyclohexylisochroman-4-yl)phenyl)-4-(dimethoxymethyl)piperidine (117 mg, 211 μmol, 69.3%) as a sticky solid whose provisionally interpreted spectra are consistent with the assigned structure. ¹H NMR (500 MHZ, CDCl₃) δ 7.44-7.29 (m, 6H), 7.12 (d, J=8.1 Hz, 2H), 6.93 (d, J=8.1 Hz, 1H), 6.81 (d, J=8.1 Hz, 1H), 6.74 (dd, J=8.6, 2.6 Hz, 1H), 6.65 (s, 1H), 4.98 (d, J=2.3 Hz, 4H), 4.82 (t, J=13.8 Hz, 1H), 4.09 (dd, J=16.2, 7.4 Hz, 1H), 3.82 (s, 1H), 3.66 (d, J=12.2 Hz, 2H), 3.43-3.33 (m, 8H), 2.60 (s, 2H), 2.06 (d, J=13.1 Hz, 1H), 1.83 (d, J=13.2 Hz, 3H), 1.68-1.59 (m, 3H), 1.43 (d, J=12.2 Hz, 2H), 1.17-1.04 (m, 5H).

### Intermediate 32. 1-(4-(7-(benzyloxy)-3-(3-methoxyphenyl)isochroman-4-yl)phenyl)-4-(dimethoxymethyl)piperidine

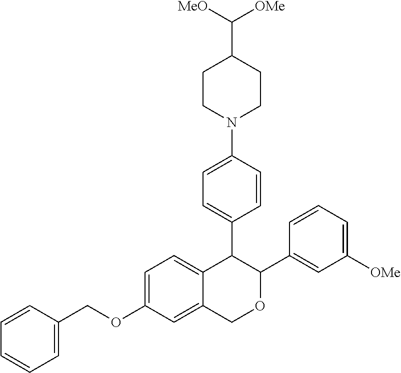 [SMILES: COc1cccc(C2OCc3cc(OCc4ccccc4)ccc3C2c2ccc(N3CCC(C(OC)OC)CC3)cc2)c1]

¹H NMR (500 MHZ, CDCl₃) § 7.46-7.31 (m, 6H), 7.16-7.03 (m, 2H), 6.84-6.56 (m, 8H), 5.16 (d, J=15.3 Hz, 1H), 5.06 (d, J=3.1 Hz, 2H), 5.04 (d, J=5.5 Hz, 1H), 4.99 (s, 1H), 4.07 (dd, J=15.6, 7.1 Hz, 1H), 3.99 (d, J=14.3 Hz, 1H), 3.58 (d, J=3.6 Hz, 5H), 3.36 (dd, J=8.3, 2.9 Hz, 6H), 2.51 (d, J=13.8 Hz, 2H), 1.79 (d, J=13.1 Hz, 2H), 1.69 (s, 1H), 1.39 (d, J=12.6 Hz, 2H).

### Bn Deprotection

### Intermediate 33. 4-(4-(4-(dimethoxymethyl)piperidin-1-yl)phenyl)-3-(4-fluorophenyl)isochroman-7-ol

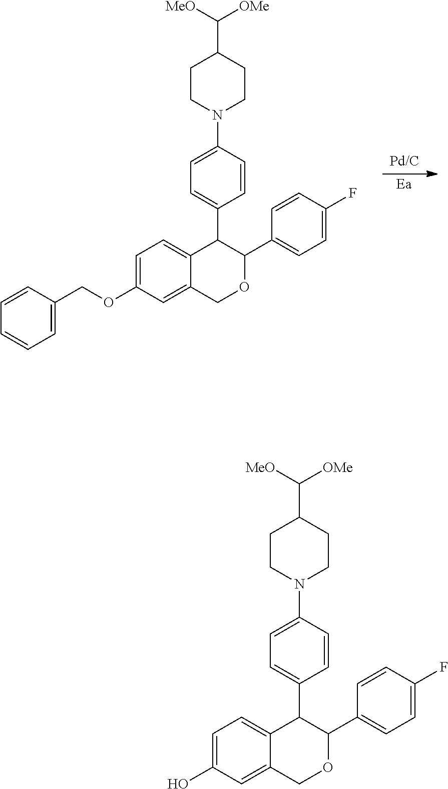 [SMILES: COC(OC)C1CCN(c2ccc(C3c4ccc(OCc5ccccc5)cc4COC3c3ccc(F)cc3)cc2)CC1 ; COC(OC)C1CCN(c2ccc(C3c4ccc(O)cc4COC3c3ccc(F)cc3)cc2)CC1]

A solution of 1-(4-(7-(benzyloxy)-3-(4-fluorophenyl)isochroman-4-yl)phenyl)-4-(dimethoxymethyl)piperidine (80 mg, 1 Eq, 0.14 mmol) in ethyl acetate was sparged with nitrogen and Pd—C (75 mg, 10% Wt, 0.5 Eq, 70 μmol) was added, the reaction vessel was capped and evacuated and filled with hydrogen from a balloon a few times, and was allowed to stir at room temperature for 3 hours, a period during which the reaction was judged complete by LCMS. The reaction mixture was sparged with nitrogen, and filtered through a short pad of celite, and concentrated in vacuo. The residue was purified by silica gel chromatography, 24 g column, dry-loaded on silica gel, and eluted with Ea, Hptn, 0-50%. The selected fractions were combined and concentrated in vacuo to give 4-(4-(4-(dimethoxymethyl)piperidin-1-yl)phenyl)-3-(4-fluorophenyl)isochroman-7-ol (38 mg, 80 μmol, 56%) as white solid whose provisionally interpreted spectra re consistent with the assigned structure. ¹H NMR (400 MHZ, CDCl₃) δ 6.95-6.78 (m, 5H), 6.74-6.47 (m, 6H), 5.13 (d, J=15.3 Hz, 1H), 5.02 (d, J=15.3 Hz, 1H), 4.98 (d, J=3.2 Hz, 1H), 4.66 (s, 1H), 4.05 (d, J=7.3 Hz, 1H), 3.93 (d, J=3.2 Hz, 1H), 3.56 (d, J=12.0 Hz, 2H), 3.35 (s, 6H), 2.54 (t, J=12.3 Hz, 2H), 1.79 (d, J=13.1 Hz, 2H), 1.68 (ddd, J=11.4, 7.6, 3.8 Hz, 1H), 1.50-1.34 (m, 2H).

### Intermediate 34. 3-cyclohexyl-4-(4-(4-(dimethoxymethyl)piperidin-1-yl)phenyl)isochroman-7-ol

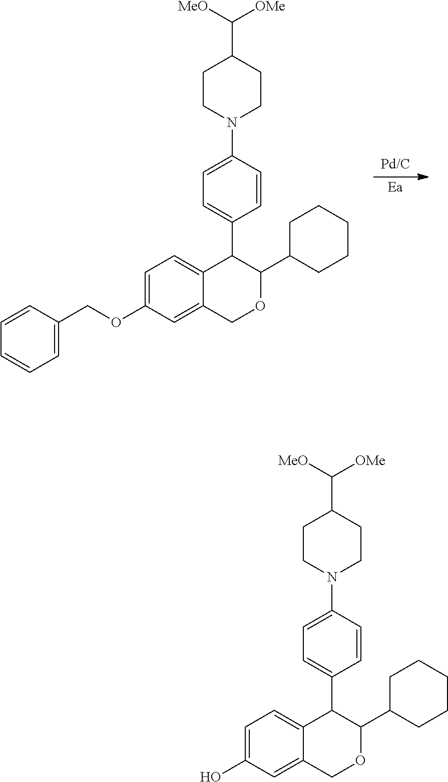 [SMILES: COC(OC)C1CCN(c2ccc(C3c4ccc(OCc5ccccc5)cc4COC3C3CCCCC3)cc2)CC1 ; COC(OC)C1CCN(c2ccc(C3c4ccc(O)cc4COC3C3CCCCC3)cc2)CC1]

A solution of 1-(4-(7˜(benzyloxy)-3-cyclohexylisochroman-4-yl)phenyl)-4-(dimethoxymethyl)piperidine (117 mg, 1 Eq, 211 μmol) in ethyl acetate was sparged with nitrogen and Pd—C (112 mg, 10% Wt, 0.5 Eq, 105 μmol) was added, the reaction vessel was capped and evacuated and filled with hydrogen from a balloon a few times, and was allowed to stir at room temperature for 3 hours, a period during which the reaction was judged complete by LCMS. The reaction mixture was sparged with nitrogen, and filtered through a short pad of celite, and concentrated in vacuo. The residue was purified by silica gel chromatography, 24 g column, dry-loaded on silica gel, and eluted with Ea, Hptn, 0-50%. The selected fractions were combined and concentrated in vacuo to give 3-cyclohexyl-4-(4-(4-(dimethoxymethyl)piperidin-1-yl)phenyl)isochroman-7-ol (55 mg, 0.12 mmol, 56%) as white solid whose provisionally interpreted spectra re consistent with the assigned structure. ¹H NMR (500 MHZ, CDCl₃) δ 7.16 (d, J=8.0 Hz, 2H), 6.92 (q, J=6.3 Hz, 1H), 6.85 (d, J=8.1 Hz, 2H), 6.61 (d, J=8.6 Hz, 1H), 6.55 (s, 1H), 5.01 (d, J=15.2 Hz, 1H), 4.84 (t, J=13.9 Hz, 1H), 4.64 (s, 1H), 4.13 (dd, J=15.9, 7.4 Hz, 1H), 3.85 (s, 1H), 3.70 (d, J=12.3 Hz, 2H), 3.51-3.38 (m, 8H), 2.62 (d, J=31.7 Hz, 2H), 2.21-2.04 (m, 1H), 1.88 (d, J=12.8 Hz, 3H), 1.82-1.62 (m, 4H), 1.48 (d, J=12.6 Hz, 2H), 1.23-1.06 (m, 5H).

### Intermediate 35. 4,4-difluoro-6-methoxy-3,4-dihydronaphthalen-1 (2H)-one

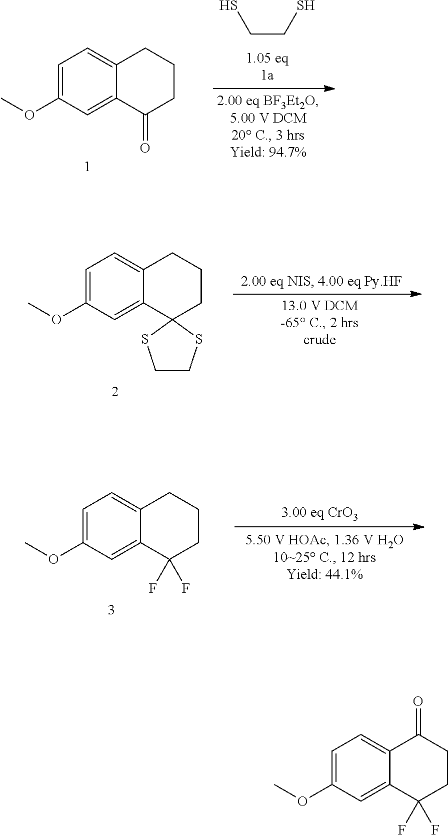

Step 1. To a mixture of compound 1 (280 g, 1.59 mol, 1.00 eq) and compound 1a (157 g, 1.67 mol, 140 mL, 1.05 eq) in DCM (1.40 L) was added BF₃·Et₂O (960 g, 3.18 mol, 834 mL, 47.0% purity, 2.00 eq) at 20° C. under N₂. The mixture was stirred at 20° C. for 3 hrs. The mixture was quenched with saturated NaHCO₃ aqueous solution (3.00 L). The aqueous was extracted with DCM (1.00 L*3), the combined organic layer was washed with brine (500 mL*2), dried over Na₂SO₄, filtered and concentrated under reduced pressure. The residue was purified by column chromatography (SiO₂, Petroleum ether:Ethyl acetate=1:0 to 10:1) and the fraction was concentrated under reduced pressure to compound 2 (380 g, 1.51 mol, 94.7% yield) as a white solid which was confirmed by ¹H NMR.

LCMS: RT=0.864 min, m/z=253.0 (M+H)⁺.

¹H NMR: 400 MHZ, CDCl₃ δ 7.49 (d, J=2.8 Hz, 1H), 6.92 (d, J=8.4 Hz, 1H), 6.73 (dd, J=8.8, 2.4 Hz, 1H), 3.81 (s, 3H), 3.69-3.54 (m, 2H), 3.53-3.41 (m, 2H), 2.74 (t, J=6.4 Hz, 2H), 2.44-2.34 (m, 2H), 2.04-1.92 (m, 2H).

Step 2. To a solution of NIS (285 g, 1.27 mol, 2.00 eq) in DCM (1.60 L) was added Py. HF (359 g, 2.54 mol, 326 mL, 70% purity, 4.00 eq) at −65° C. under N₂. The mixture was stirred at −65° C. for 30 mins. A solution of compound 2 (160 g, 634 mmol, 1.00 eq) dissolved in DCM (500 mL) was added to the mixture at −65° C., then the mixture was stirred at −65° C. for 1.5 hrs. The mixture was poured into saturated NaHCO₃ aqueous solution (3.00 L) under stirring, the aqueous phase was adjusted pH value to 7 with saturated NaHCO₃ aqueous solution. The mixture was quenched with saturated Na₂SO₃ aqueous solution (2.00 L). The mixture was extracted with DCM (1.00 L*3), then combined organic layer was washed with HCl (1M, 500 mL), brine (500 mL*2), dried with Na₂SO₄, filtered, and concentrated under reduced pressure to obtain compound 3 (133 g, crude) as yellow oil which was confirmed by ¹H NMR and F NMR.

¹H NMR: 400 MHZ, CDCl₃ δ 7.17 (d, J=2.8 Hz, 1H), 7.08 (d, J=8.8 Hz, 1H), 6.98-6.89 (m, 1H), 3.83 (s, 3H), 2.78-2.75 (m, 2H), 2.37-2.17 (m, 2H), 2.04-1.93 (m, 2H).

Step 3. To a solution of compound 3 (110 g, 555 mmol, 1.00 eq) in AcOH (600 mL) was added a solution of CrO₃ (166 g, 1.66 mol, 61.7 mL, 3.00 eq) in H₂O (150 mL) at 10° C. The mixture was warmed to 25° C. and stirred at 25° C. for 12 hrs. The mixture was poured into H₂O (2.00 L) under stirring, then the aqueous phase was adjusted pH value to 7 with saturated NaHCO₃ aqueous solution. The mixture was extracted with ethyl acetate (1.50 L*3), then combined organic layer was washed with brine (500 mL*2), dried with Na₂SO₄, filtered, concentrated under reduced pressure. The residue was dissolved in Petroleum ether:Ethyl acetate (1:1, 1.50 L). The mixture was filtered through SiO₂ and the fraction was concentrated under reduced pressure. The crude product was triturated with Petroleum ether:Ethyl acetate (20:1, 100 mL) at 20° C. for 30 mins and filtered under reduced pressure to obtain 4,4-difluoro-6-methoxy-3,4-dihydronaphthalen-1 (2H)-one (53.0 g, 244 mmol, 44.1% yield, 97.9% purity) as a yellow solid which was confirmed by ¹H NMR, F NMR, LCMS and HPLC.

LCMS: RT=0.704 min, m/z=213.0 (M+H)⁺.

¹H NMR: 400 MHZ, CDCl₃ δ 8.01 (d, J=8.4 Hz, 1H), 7.19 (d, J=2.4 Hz, 1H), 7.07 (dd, J=8.4, 2.4 Hz, 1H), 3.92 (s, 3H), 2.91-2.78 (m, 2H), 2.69-2.56 (m, 2H).

### Example 1. Preparation of (3S)-3-[5-[4-[[1-[4-[(1S,2S)-2-cyclohexyl-6-hydroxy-tetralin-1-yl]phenyl]-4-piperidyl]methyl]piperazin-1-yl]-1-oxo-isoindolin-2-yl]piperidine-2,6-dione (1-5) and (3S)-3-[5-[4-[[1-[4-[(1R,2R)-2-cyclohexyl-6-hydroxy-tetralin-1-yl]phenyl]-4-piperidyl]methyl]piperazin-1-yl]-1-oxo-isoindolin-2-yl]piperidine-2,6-dione (I-6)

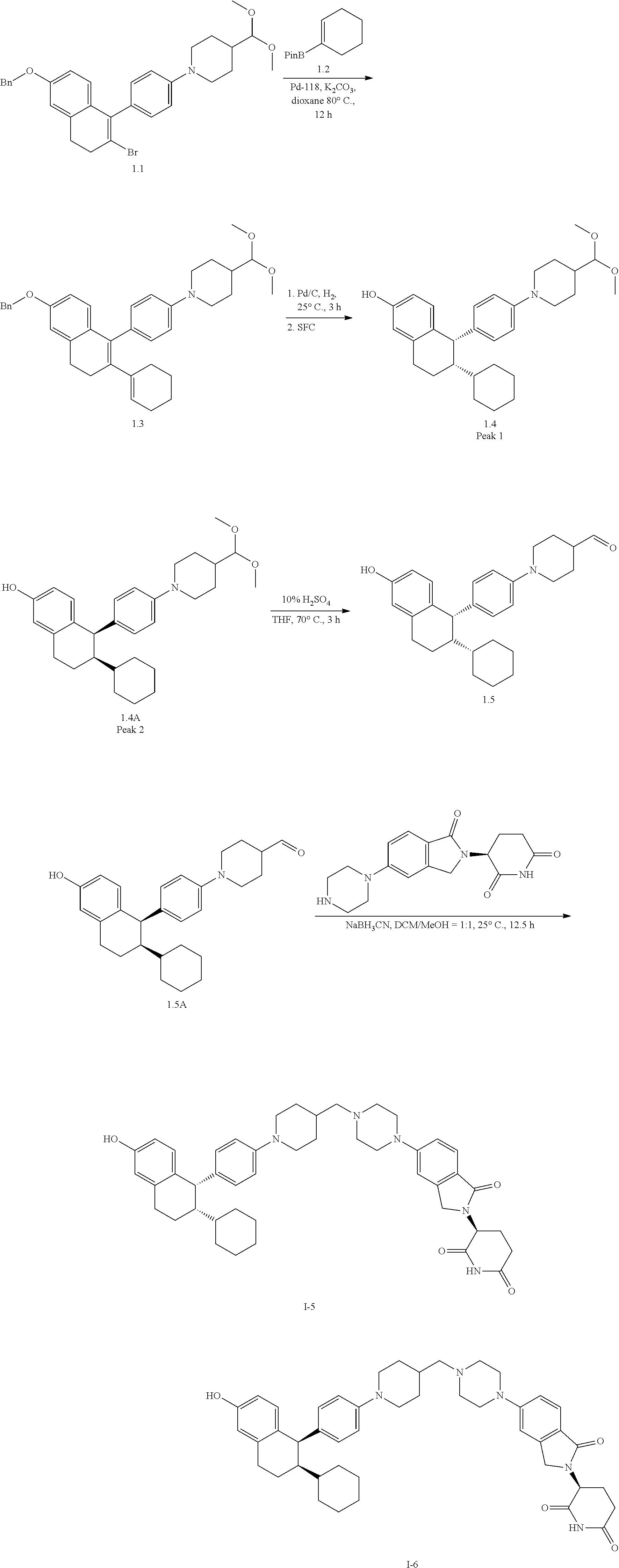

Step 1: A mixture of 1.1 (800 mg, 1.46 mmol), 1.2 (334 mg, 1.60 mmol), 1,1′-Bis(di-t-butylphosphino) ferrocene palladium dichloride (95.1 mg, 146 μmol) and Na₂CO₃ (309 mg, 2.92 mmol) in 1,4-dioxane (10 mL) and H₂O (2 mL) was degassed and purged with N₂ for 3 times, and then the mixture was stirred at 80° C. for 12 h under N₂ atmosphere. The mixture was diluted with water (20 mL) and extracted with ethyl acetate (30 mL×3). The combined organic phases were washed with brine (30 mL), dried over anhydrous Na₂SO₄, filtered and concentrated under reduced pressure to give a residue. The residue was purified by flash silica gel chromatography (ethyl acetate in petroleum ether=0% to 25%) to give 1.3 (450 mg, 53.0% yield) as a yellow solid. LC-MS (ESI⁺) m/z: 550.5 (M+H)⁺.

Step 2: To a solution of Pd/C (400 mg, 10.0% purity, 376 μmol) in MeOH (15 mL) was added 1.3 (450 mg, 794 μmol) under N₂. The suspension was degassed under vacuum and purged with Hz several times. The mixture was stirred under H₂ (15 psi) at 25° C. for 3 h. The reaction mixture was filtered and the filter was concentrated to give a residue which was separated by chiral SFC (column: DAICEL CHIRALCEL OJ (250 mm*30 mm, 10 um); mobile phase: [0.1% NH₃H₂O EtOH]; B %: 30%-30%, Flow Rate (80 ml/min) peak 1:1.835 min, peak 2:2.088 min) to yield Peak 1 and Peak 2. Peak 1 was concentrated under reduced pressure to yield 1.4 (120 mg, 29.9% yield) as a yellow solid. LC-MS (ESI⁺) m/z: 464.4 (M+H)⁺.

Peak 2 was concentrated under reduced pressure to yield 1.4 A (120 mg, 29.9% yield) as a yellow solid. LC-MS (ESI⁺) m/z: 464.4 (M+H)⁺.

Step 3: To a solution of 1.4 (120 mg, 258.82 μmol) in THF (3 mL) was added H₂SO₄ (3 mL, 5.63 mmol, 10.0% purity). The mixture was stirred at 70° C. for 3 h. The reaction mixture was quenched by addition of water (10 mL), adjust pH to 8 by NaHCO₃ saturated solution, extracted with EtOAc (20 mL×3). The combined organic layers were washed with brine (10 mL), dried over Na₂SO₄, filtered and concentrated under reduced pressure to give 1.5 (130 mg, 96.23% yield) as a yellow solid. LC-MS (ESI⁺) m/z: 418.4 (M+H)⁺.

Step 3A: To a solution of 1.4 A (120 mg, 258.82 μmol) in THE (3 mL) was added H₂SO₄ (3 mL, 5.63 mmol, 10.0% purity). The mixture was stirred at 70° C. for 3 h. The reaction mixture was quenched by addition of water (10 mL), adjust pH to 8 by NaHCO₃ saturated solution, extracted with EtOAc (20 mL×3). The combined organic layers were washed with brine (10 mL), dried over Na₂SO₄, filtered and concentrated under reduced pressure to give 1.5 A (130 mg, 96.2% yield) as a yellow solid. LC-MS (ESI⁺) m/z: 418.4 (M+H)⁺.

Step 4: To a solution of 1.5 (60 mg, 114.95 μmol) and (3S)-3-(1-oxo-5-piperazin-1-yl-isoindolin-2-yl)piperidine-2,6-dione (37.74 mg, 114.95 μmol) in DCM (2.5 mL) and MeOH (2.5 mL) after stirred 30 min was added sodium cyanoboranuide (36.12 mg, 574.74 μmol). The mixture was stirred at 25° C. for 12.5 h. The reaction mixture was concentrated to yield a residue which was purified by preparative HPLC (column: Boston Green ODS 150*30 mm*5 um; mobile phase: [water(FA)-ACN]; B %: 27%-47%, 12 min), followed by lyophilization to give (3S)-3-[5-[4-[[1-[4-[(1S,2S)-2-cyclohexyl-6-hydroxy-tetralin-1-yl]phenyl]-4-piperidyl]methyl]piperazin-1-yl]-1-oxo-isoindolin-2-yl]piperidine-2,6-dione, I-5, (30 mg, 35.0% yield, 0.1FA) as a white solid.

LC-MS (ESI+) m/z: 730.4 (M+H)⁺. LCMS: calc. for C₄₅H₅₅N₅O₄:729.4, found: [M+H]⁺730.4.

HPLC: 100% purity at 254 nm.

¹H NMR (500 MHz, MeOD-d4): δ 8.10 (s, 1H), 7.70 (d, J=9.0 Hz, 1H), 7.67 (s, 1H), 7.21-7.14 (m, 2H), 6.98-6.87 (m, 4H), 6.65 (d, J=8.4 Hz, 1H), 6.55 (d, J=2.1 Hz, 1H), 6.45 (dd, J=2.4, 8.3 Hz, 1H), 5.12 (dd, J=5.2, 13.4 Hz, 1H), 4.50-4.36 (m, 2H), 4.14 (d, J=4.4 Hz, 1H), 3.81-3.51 (m, 4H), 3.41 (s, 5H), 3.10 (d, J=6.4 Hz, 2H), 2.95-2.86 (m, 2H), 2.84-2.70 (m, 4H), 2.47 (d, J=4.5, 13.1 Hz, 1H), 2.18-2.09 (m, 2H), 2.05 (s, 1H), 1.92 (d, J=12.2 Hz, 2H), 1.84-1.72 (m, 2H), 1.67-1.55 (m, 4H), 1.55-1.45 (m, 2H), 1.34-0.96 (m, 7H), 0.85-0.73 (m, 1H).

Step 4A: To a solution of 1.5 A (60 mg, 114.95 μmol) and (3S)-3-(1-oxo-5-piperazin-1-yl-isoindolin-2-yl)piperidine-2,6-dione (37.74 mg, 114.95 μmol) in DCM (2.5 mL) and MeOH (2.5 mL) after stirred 30 min was added sodium cyanoboranuide (36.12 mg, 574.74 μmol). The mixture was stirred at 25° C. for 12.5 h. The reaction mixture was concentrated to yield a residue which was purified by preparative HPLC (column: Boston Green ODS 150*30 mm*5 um; mobile phase: [water(FA)-ACN]; B %: 27%-47%, 12 min), followed by lyophilization to give (3S)-3-[5-[4-[[1-[4-[(1R,2R)-2-cyclohexyl-6-hydroxy-tetralin-1-yl]phenyl]-4-piperidyl]methyl]piperazin-1-yl]-1-oxo-isoindolin-2-yl]piperidine-2,6-dione, I-6, (30.6 mg, 36.3% yield, 0.06FA) as a white solid.

LC-MS (ESI+) m/z: 730.4 (M+H)⁺. LCMS: calc. for C₄₅H₅₅N₅O₄:729.4, found: [M+H]⁺ 730.4.

HPLC: 100% purity at 254 nm.

1H NMR (500 MHz, MeOD-d4): δ 8.09 (s, 1H), 7.73-7.66 (m, 1H), 7.22-7.15 (m, 2H), 6.91 (q, J=8.6 Hz, 4H), 6.65 (d, J=8.4 Hz, 1H), 6.55 (d, J=2.1 Hz, 1H), 6.45 (dd, J=2.4, 8.3 Hz, 1H), 5.12 (dd, J=5.1, 13.4 Hz, 1H), 4.51-4.36 (m, 2H), 4.14 (d, J=4.3 Hz, 1H), 3.68 (s, 4H), 3.45 (s, 5H), 3.17-3.08 (m, 2H), 2.95-2.85 (m, 2H), 2.82-2.70 (m, 4H), 2.47 (d, J=4.5, 13.2 Hz, 1H), 2.27-2.11 (m, 2H), 2.05 (d, J=11.3 Hz, 1H), 1.92 (d, J=12.5 Hz, 2H), 1.81-1.72 (m, 2H), 1.71-1.37 (m, 7H), 1.35-0.98 (m, 7H), 0.79 (q, J=10.9 Hz, 1H).

### Example 2. Preparation of (I-13) S)-3-(5-(4-((1-(4-((1S,2R)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

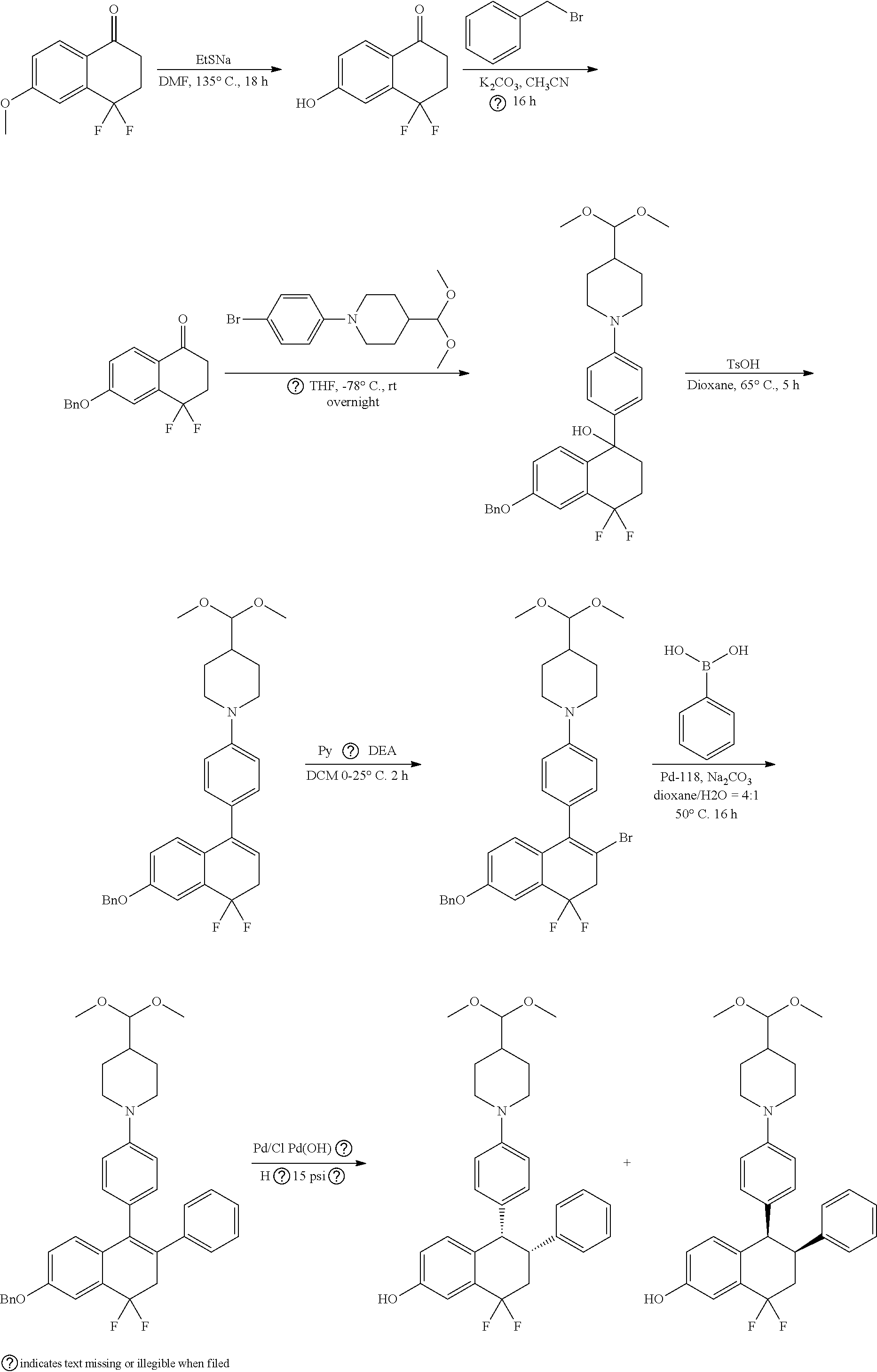

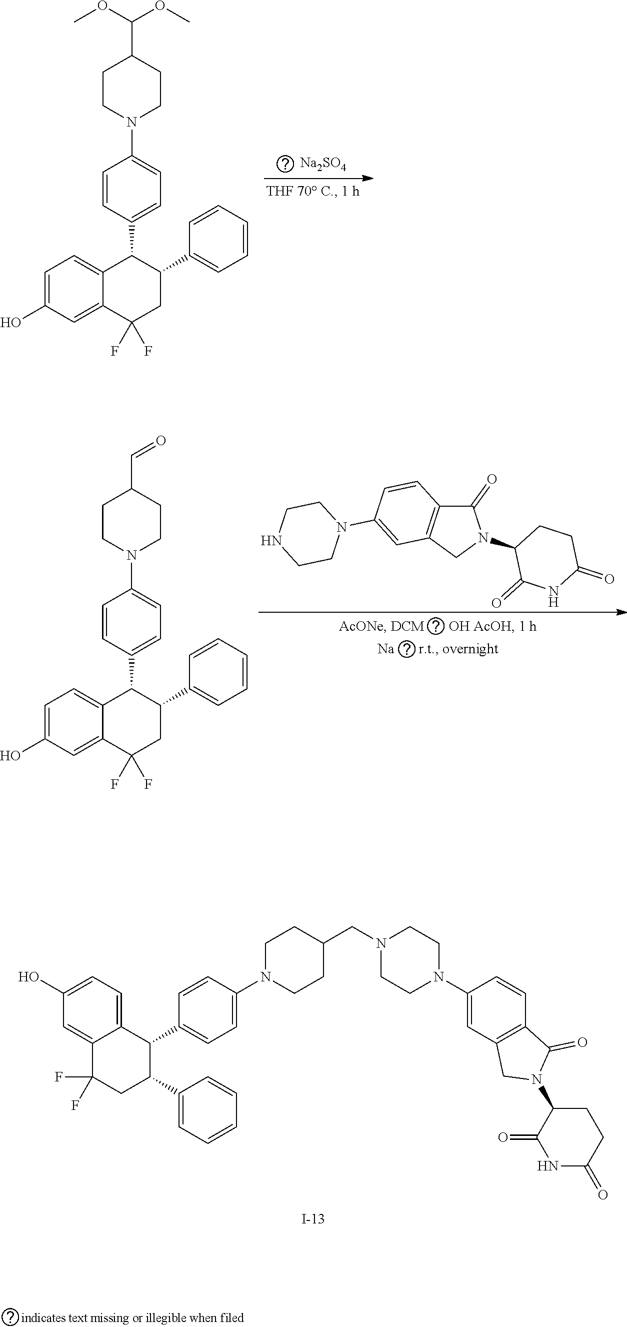

Step 1: To a solution of 4,4-difluoro-6-methoxy-3,4-dihydronaphthalen-1 (2H)-one (5.3 g, 25 mmol) in DMF (100 mL) at r.t. was added EtSNa (8.4 g, 0.1 mol) with N₂. After addition, the mixture was stirred at 135° C. for 16 h with N₂. The reaction mixture was added H₂O (200 mL) and extracted with ethyl acetate (200 mL×2). The combined organic layers were dried over Na₂SO₄, filtered and concentrated under reduced pressure to give a residue. The residue was purified by flash silica gel chromatography (ethyl acetate in petroleum ether=0% to 20%) to give the product of 4,4-difluoro-6-hydroxy-3,4-dihydronaphthalen-1 (2H)-one (4.1 g, 73% yield) as a yellow solid. LC-MS (ESI⁺) m/z: 199.1 (M+H)⁺.

Step 2: To a solution of 4,4-difluoro-6-hydroxy-3,4-dihydronaphthalen-1 (2H)-one (4.1 g, 18 mmol) in CH₃CN (50 mL) at r.t. was added (bromomethyl)benzene (3.7 g, 22 mmol) and K₂CO₃ (5.0 g, 36 mmol) with N₂. After addition, the mixture was stirred at 25° C. for 16 h. The mixture was treated with H₂O (150 mL), extracted with EtOAc (150 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 6-(benzyloxy)-4,4-difluoro-3,4-dihydronaphthalen-1 (2H)-one (5 g, 100% yield,) as a white solid. LC-MS (ESI⁺) m/z: 289.1 (M+H)⁺.

Step 3: To a solution of 1-(4-bromophenyl)-4-(dimethoxymethyl)piperidine (7 g, 0.02 mol) in THF (60 mL) was added dropwise n-BuLi (2 g, 2.5 M, 0.03 mol) at −78° C. with N₂. After addition, the mixture was stirred at this temperature for 1 h, then 6-(benzyloxy)-4,4-difluoro-3,4-dihydronaphthalen-1 (2H)-one (5 g, 0.02 mol) in THF (20 mL) was added dropwise at −78° C. The resulting mixture was stirred at −78° C.-r.t. for 16 h. The reaction mixture was quenched into a solution of saturated ammonium chloride solution followed by extraction with EtOAc (100 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 6-(benzyloxy)-1-(4-(4-(dimethoxymethyl)piperidin-1-yl)phenyl)-4,4-difluoro-1,2,3,4-tetrahydronaphthalen-1-ol (6.37 g, 60% yield) as a white solid. LC-MS (ESI+) m/z: 524.3 (M+H)⁺.

Step 4: A mixture of 6-(benzyloxy)-1-(4-(4˜(dimethoxymethyl)piperidin-1-yl)phenyl)-4,4-difluoro-1,2,3,4-tetrahydronaphthalen-1-ol (6.37 g, 10.6 mmol) in Dioxane (80 mL) at r.t., then TsOH (202 mg, 1.06 mmol) was added to the mixture and was stirred at 65° C. for 5 h. The mixture was treated with H₂O (300 mL), extracted with EtOAc (300 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-4,4-difluoro-3,4-dihydronaphthalen-1-yl)phenyl)-4-(dimethoxymethyl)piperidine (4.6 g, 86% yield) as a yellow solid. LC-MS (ESI⁺) m/z: 506.3 (M+H)⁺.

Step 5: To a mixture of 1-(4-(6-(benzyloxy)-4,4-difluoro-3,4-dihydronaphthalen-1-yl)phenyl)-4-(dimethoxymethyl)piperidine (1.28 g, 2.30 mmol) and Diisopropylethylamine (446 mg, 3.45 mmol) in DCM (15 mL) at 0° C., and Pyridinium bromide perbromide (809 mg, 2.53 mmol) was added to the mixture at 0° C., then the mixture was stirred at 0° C. for 4 h. The mixture was concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-2-bromo-4,4-difluoro-3,4-dihydronaphthalen-1-yl)phenyl)-4-(dimethoxymethyl)piperidine (1.36 g, 47.0% yield) as a yellow solid. LC-MS (ESI+) m/z: 586.1 (M+H)⁺.

Step 6:1,1′-Bis(di-t-butylphosphino)ferrocene palladium dichloride (70.5 mg, 108 μmol) was added to the mixture of 1-(4-(6-(benzyloxy)-2-bromo-4,4-difluoro-3,4-dihydronaphthalen-1-yl)phenyl)-4-(dimethoxymethyl)piperidine (1.36 g, 1.08 mmol), phenylboronic acid (198 mg, 1.62 mmol) and Sodium carbonate (344 mg, 3.24 mmol) in Dioxane (20 mL) and H₂O (5 mL) at r.t. N₂ was bubbled into the mixture for 5 min. The reaction mixture was heated at 50° C. for 12 h. The mixture was treated with H₂O (150 mL), extracted with EtOAc (150 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-4,4-difluoro-2-phenyl-3,4-dihydronaphthalon-1-yl)phenyl)-4-(dimethoxymethyl)piperidine (1.15 g, 88.6% yield) as a yellow solid. LC-MS (ESI+) m/z: 582.5 (M+H)⁺.

Step 7: A mixture of 1-(4-(6-(benzyloxy)-4,4-difluoro-2-phenyl-3,4-dihydronaphthalen-1-yl)phenyl)-4-(dimethoxymethyl)piperidine (950 mg, 792 μmol), Pd/C (500 mg, 470 μmol, 10% wt), Pd(OH)₂ (500 mg, 712 μmol, 20% wt) in MeOH (15 mL) was degassed and purged with H₂ for 3 times, and then the mixture was stirred at 25° C. for 16 h under H₂ atmosphere (15 psi). The reaction mixture was filtered and concentrated under reduced pressure to give a residue. The residue was purified by prep-HPLC (column: Xtimate C18 150*40 mm*5 um; mobile phase: [water(FA)-ACN]; B % 55%-95%, 8 min) and was further separated by SFC (condition: column: DAICEL CHIRALPAK AD (250 mm*30 mm, 10 um); mobile phase: [Neu-ETOH]; B %: 30%-30%, min) to give (5S, 6R)-5-(4-(4-(dimethoxymethyl)piperidin-1-yl)phenyl)-8,8-difluoro-6-phenyl-5,6,7,8-tetrahydronaphthalen-2-ol (100 mg, 25.4% yield) and (5R, 6S)5-(4-(4-(dimethoxymethyl)piperidin-1-yl)phenyl)-8,8-difluoro-6-phenyl-5,6,7,8-tetrahydronaphthalen-2-ol (110 mg, 27.7% yield) as yellow solid. LC-MS (ESI+) m/z: 494.3, 494.2 (M+H)⁺.

Step 8: A mixture of (5S, 6R)-5-(4-(4-(dimethoxymethyl)piperidin-1-yl)phenyl)-8,8-difluoro-6-phenyl-5,6,7,8-tetrahydronaphthalen-2-ol (50 mg, 0.10 mmol) in THF (3.5 mL) and 10% H₂SO₄ (3.5 mL) was stirred at 70° C. for 1 h. The reaction was adjust to pH=8 with aq. NaHCO₃ (10 mL). Then the mixture was added water (20 mL) and extracted with ethyl acetate (2×20 mL). The combined organic layers were washed with brine (20 mL), dried over anhydrous Na₂SO₄, filtered and concentrated to dryness in vacuo to give a yellow solid of 1-(4-((1S,2R)-4,4-difluoro-6-hydroxy-2-phenyl-1,2,3,4-tetrahydronaphthalen-1-yl)phenyl)piperidine-4-carbaldehyde (45 mg, 97% yield). LC-MS (ESI+) m/z: 466.3 (M+H)⁺.

Step 9: A mixture of 1-(4-((1S,2R)-4,4-difluoro-6-hydroxy-2-phenyl-1,2,3,4-tetrahydronaphthalen-1-yl)phenyl)piperidine-4-carbaldehyde (45 mg, 98 μmol), (S)-3-(1-oxo-5-(piperazin-1-yl)isoindolin-2-yl)piperidine-2,6-dione, TsOH salt (0.12 g, 0.15 mmol) and acetic acid (12 mg, 0.20 mmol) and sodium acetate (40 mg, 0.49 mmol) in THF (1 mL) and MeOH (1 mL) at 25° C. for 1 h, then sodium triacetoxyborohydride (41 mg, 0.20 mmol) was added to the mixture and was stirred at 25° C. for 16 h. Then the mixture was added water (10 mL) and extracted with ethyl acetate (2×15 mL). The combined organic layers were washed with brine (20 mL), dried over anhydrous Na₂SO₄, filtered and concentrated to dryness in vacuo to give a residue. The residue was purified by prep-HPLC (column: O-Welch C18 150*30 mm*5 um; mobile phase: [water(FA)-ACN]; B % 10%-50%, 10 min) to give (S)-3-(5-(4-((1-(4-((1S,2R)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (19.2 mg, 26% yield). LC-MS (ESI+) m/z: 760.4 (M+H)⁺.

LCMS: calc. for C₄₅H₄₇F₂N₅O₄: 759.36, found: [M+H]⁺ 760.4.

HPLC: 100% purity at 220 nm.

¹HNMR (400 MHz, DMSO-d₆) δ=10.95 (s, 1H), 9.73 (s, 1H), 8.14 (s, 1H), 7.61-7.47 (m, 1H), 7.26-7.17 (m, 3H), 7.09 (br s, 3H), 6.95-6.83 (m, 4H), 6.58 (br d, J=8.0 Hz, 2H), 6.14 (br d, J=8.1 Hz, 2H), 5.06 (dd, J=5.1, 13.1 Hz, 1H), 4.38-4.28 (m, 2H), 4.27-4.17 (m, 1H), 3.67 (br d, J=15.7 Hz, 1H), 3.53 (br s, 3H), 3.31-3.30 (m, 4H), 2.96-2.84 (m, 1H), 2.61 (br s, 9H), 2.37 (br d, J=8.9 Hz, 2H), 2.01-1.93 (m, 1H), 1.77 (br d, J=11.4 Hz, 3H), 1.19 (br s, 2H)

SFC: retention time, 2.682 min; Area, 97.658%.

### Example 3. Preparation of (1-51) (S)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

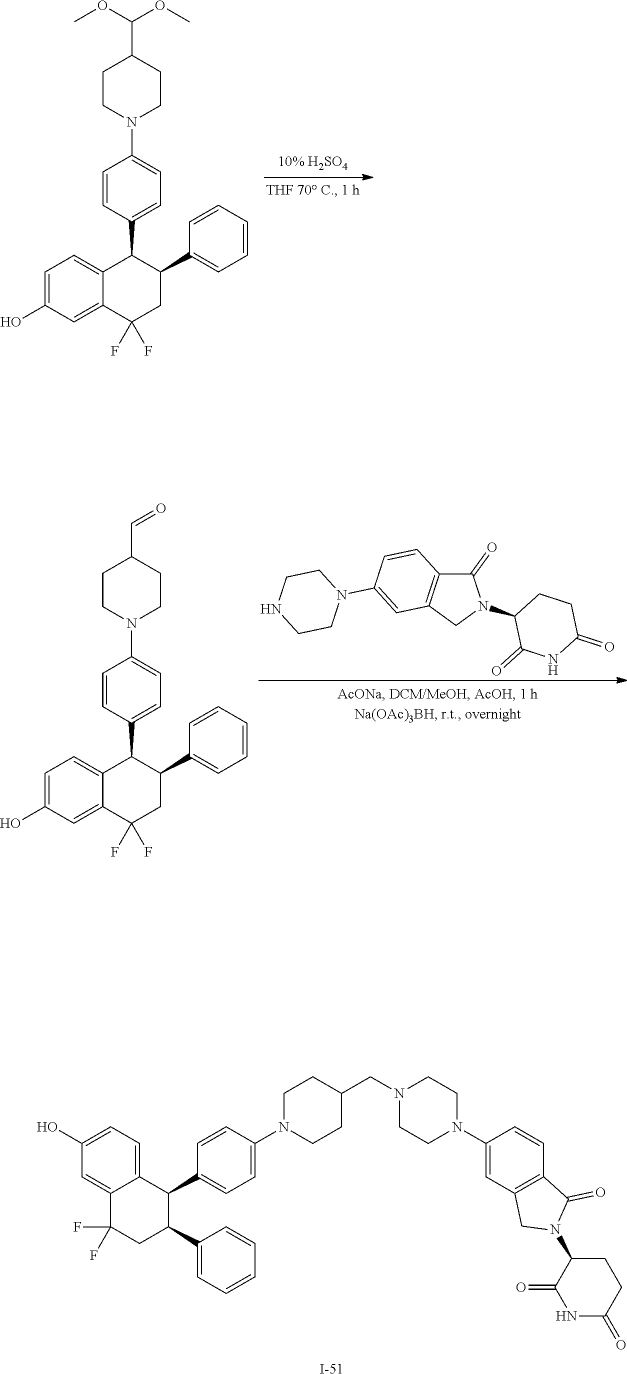

Step 1: A mixture of (5R,6S)-5-(4-(4-(dimethoxymethyl)piperidin-1-yl)phenyl)-8,8-difluoro-6-phenyl-5,6,7,8-tetrahydronaphthalen-2-ol (55 mg, 0.11 mmol) in THF (3.5 mL) and 10% H₂SO₄ (3.5 mL) was stirred at 70° C. for 1 h. The reaction was adjust to pH=8 with aq. NaHCO₃ (10 mL). Then the mixture was added water (20 mL) and extracted with ethyl acetate (2×20 mL). The combined organic layers were washed with brine (20 mL), dried over anhydrous Na₂SO₄, filtered and concentrated to dryness in vacuo to give a yellow solid of 1-(4-((1R,2S)-4,4-difluoro-6-hydroxy-2-phenyl-1,2,3,4-tetrahydronaphthalen-1-yl)phenyl)piperidine-4-carbaldehyde (50 mg, 98% yield). LC-MS (ESI+) m/z: 466.3 (M+H)⁺.

Step 2: A mixture of 1-(4-((1R,2S)-4,4-difluoro-6-hydroxy-2-phenyl-1,2,3,4-tetrahydronaphthalen-1-yl)phenyl)piperidine-4-carbaldehyde (50 mg, 0.11 mmol), (S)-3-(1-oxo-5-(piperazin-1-yl)isoindolin-2-yl)piperidine-2,6-dione, TsOH salt (0.12 g, 0.15 mmol) and Acetic acid (12 mg, 0.20 mmol) and sodium acetate (40 mg, 0.49 mmol) in THF (1 mL) and MeOH (1 mL) at 25° C. for 1 h, then sodium triacetoxyborohydride (41 mg, 0.20 mmol) was added to the mixture and was stirred at 25° C. for 16 h. Then the mixture was added water (10 mL) and extracted with ethyl acetate (2×15 mL). The combined organic layers were washed with brine (20 mL), dried over anhydrous Na₂SO₄, filtered and concentrated to dryness in vacuo to give a residue. The residue was purified by prep-HPLC (column: O-Welch C18 150*30 mm*5 um; mobile phase: [water(FA)-ACN]; B % 10%-50%, 10 min) to give (S)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (27.2 mg, 33% yield). LC-MS (ESI+) m/z: 760.4 (M+H)⁺.

LCMS: calc. for C₄₅H₄₇F₂N₃O₄: 759.36, found: [M+H]⁺ 760.4.

HPLC: 100% purity at 220 nm.

¹HNMR (400 MHz, DMSO-d₆) δ=10.96 (s, 1H), 8.19 (s, 1H), 7.52 (d, J=8.7 Hz, 1H), 7.25-7.16 (m, 3H), 7.11-7.03 (m, 3H), 6.94-6.83 (m, 4H), 6.57 (br d, J=8.8 Hz, 2H), 6.13 (d, J=8.6 Hz, 2H), 5.05 (dd, J=4.9, 13.3 Hz, 1H), 4.36-4.28 (m, 2H), 4.25-4.17 (m, 1H), 3.67 (br d, J=17.4 Hz, 1H), 3.53 (br d, J=8.6 Hz, 3H), 3.28 (br s, 7H), 3.00-2.82 (m, 1H), 2.61 (br s, 5H), 2.43-2.35 (m, 1H), 2.19 (br d, J=7.2 Hz, 2H), 2.01-1.91 (m, 1H), 1.75 (br d, J=11.3 Hz, 2H), 1.64 (br s, 1H), 1.22-1.08 (m, 2H).

SFC: retention time, 2.806 min; Area, 94.060%.

### Example 4. Preparation of (1-300) 1-(6-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-methyl-1H-indazol-3-yl)dihydropyrimidine-2,4 (1H,3H)-dione

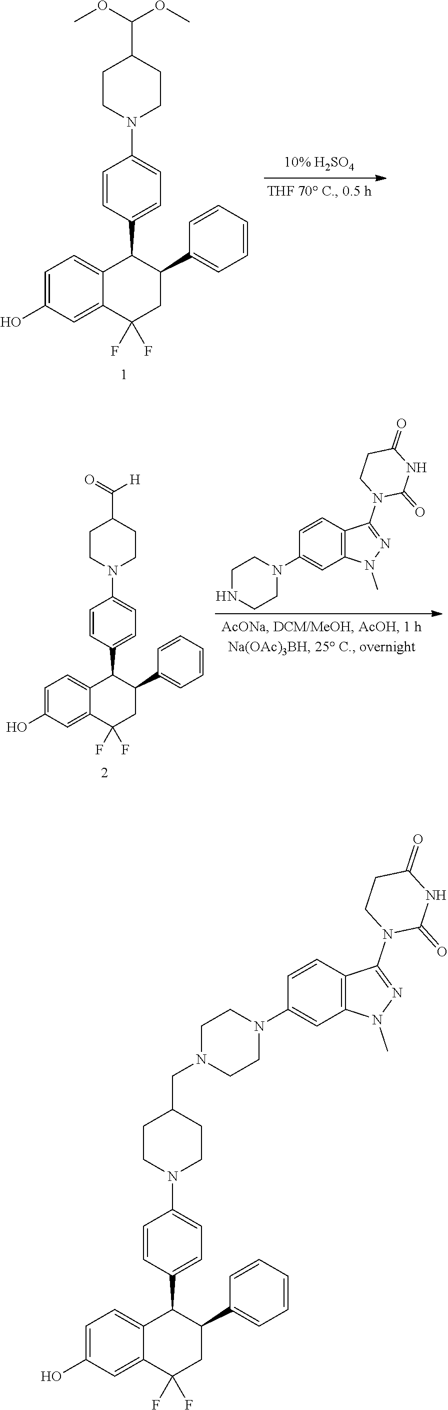 [SMILES: COC(OC)C1CCN(c2ccc([C@@H]3c4ccc(O)cc4C(F)(F)C[C@@H]3c3ccccc3)cc2)CC1 ; O=CC1CCN(c2ccc([C@@H]3c4ccc(O)cc4C(F)(F)C[C@@H]3c3ccccc3)cc2)CC1 ; Cn1nc(N2CCC(=O)NC2=O)c2ccc(N3CCNCC3)cc21 ; Cn1nc(N2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21]

Step 1: A mixture of (5R,6S)-5-(4-(4-(dimethoxymethyl)piperidin-1-yl)phenyl)-8,8-difluoro-6-phenyl-5,6,7,8-tetrahydronaphthalen-2-ol (100 mg, 97.6% purity, 1 Eq, 198 μmol) in THF (3 mL) and 10% H₂SO₄ (3 mL) was stirred at 70° C. for 0.5 h. The reaction was adjust to pH=8 with saturated aqueous NaHCO₃ (6 mL) at 0° C. Then the mixture was added H₂O (20 mL) and extracted with ethyl acetate (20 mL*2). The combined organic layers were dried over anhydrous Na₂SO₄, filtered and concentrated to dryness in vacuum to give 1-(4-((1R,2S)-4,4-difluoro-6-hydroxy-2-phenyl-1,2,3,4-tetrahydronaphthalen-1-yl)phenyl)piperidine-4-carbaldehyde (90 mg, 98% yield, 96.4% purity) as a yellow solid. LC-MS (ESI+) m/z: 448.1 (M+H)⁺

Step 2: A mixture of 1-(1-methyl-6-(piperazin-1-yl)-1H-indazol-3-yl)dihydropyrimidine-2,4 (1H,3H)-dione (50 mg, 1 Eq, 0.15 mmol), 1-(4-((1R,2S)-4,4-difluoro-6-hydroxy-2-phenyl-1,2,3,4-tetrahydronaphthalen-1-yl)phenyl)piperidine-4-carbaldehyde (78 mg, 1.1 Eq, 0.17 mmol), sodium acetate (62 mg, 5 Eq, 0.76 mmol) and acetic acid (27 mg, 26 μL, 3 Eq, 0.46 mmol) in THF (4 mL) and MeOH (4 mL) was stirred at 25° C. for 1 h, then sodium triacetoxyborohydride (65 mg, 2 Eq, 0.3 mmol) was added to the mixture and stirred at 25° C. for 16 h. Then the mixture was added H₂O (50 mL) and extracted with ethyl acetate (50 mL*2). The combined extracts were dried over anhydrous Na₂SO₄, filtered and concentrated to dryness in vacuum to give a residue. The residue was purified by prep-HPLC (column: Welch Xtimate C18 150*30 mm*5 um; mobile phase: [water(FA)-ACN]; B % 10%-60%, 8 min) to give 1-(6-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-methyl-1H-indazol-3-yl)dihydropyrimidine-2,4 (1H,3H)-dione (61.5 mg, 53% yield, 99.5% purity) as a white solid. LC-MS (ESI+) m/z: 760.3 (M+H)⁺.

LCMS: calc. for C₄₄H₄₇F₂N₇O₃: 759.37, found: [M+H]⁺ 760.3.

HPLC: 99.517% purity at 220 nm.

¹HNMR (400 MHz, DMSO-d₆) δ=10.52 (s, 1H), 9.96-9.58 (m, 1H), 8.17 (s, 0.409H), 7.45 (d, J=9.1 Hz, 1H), 7.25-7.17 (m, 3H), 7.09 (s, 1H), 6.96-6.80 (m, 6H), 6.57 (d, J=8.7 Hz, 2H), 6.13 (d, J=8.6 Hz, 2H), 4.31 (br s, 1H), 3.96-3.84 (m, 5H), 3.67 (br d, J=14.8 Hz, 1H), 3.54 (br d, J=9.4 Hz, 2H), 3.22 (br s, 3H), 2.74 (t, J=6.7 Hz, 2H), 2.70-2.52 (m, 6H), 2.50-2.46 (m, 2H), 2.32 (brt, J=15.3 Hz, 1H), 2.20 (br d, J=7.0 Hz, 2H), 1.81-1.58 (m, 3H), 1.22-1.08 (m, 2H)

SFC: retention time, 2.679 min; Area, 100%.

### Example 5. Preparation of (I-294) 1-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)-2-methylphenyl)dihydropyrimidine-2,4 (1H,3H)-dione

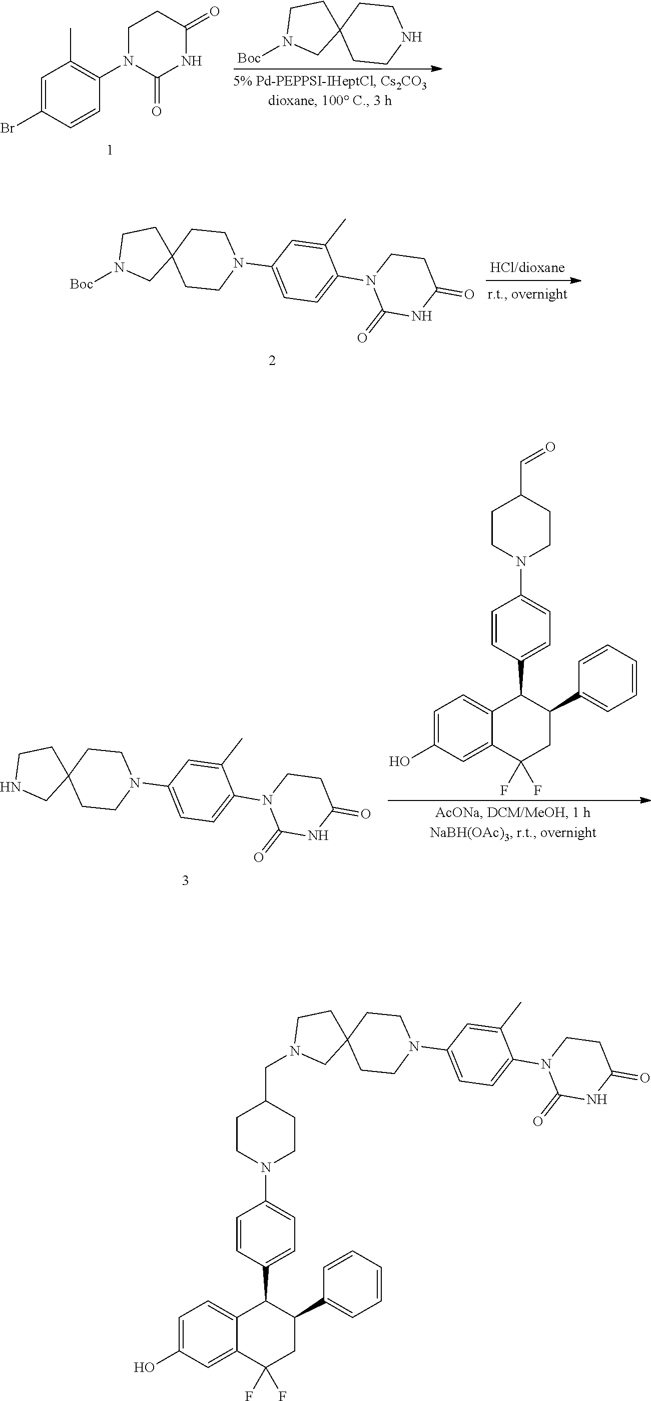 [SMILES: Cc1cc(Br)ccc1N1CCC(=O)NC1=O ; CC(C)(C)OC(=O)N1CCC2(CCNCC2)C1 ; Cc1cc(N2CCC3(CCN(C(=O)OC(C)(C)C)C3)CC2)ccc1N1CCC(=O)NC1=O ; Cc1cc(N2CCC3(CCNC3)CC2)ccc1N1CCC(=O)NC1=O ; O=CC1CCN(c2ccc([C@@H]3c4ccc(O)cc4C(F)(F)C[C@@H]3c3ccccc3)cc2)CC1 ; Cc1cc(N2CCC3(CCN(CC4CCN(c5ccc([C@@H]6c7ccc(O)cc7C(F)(F)C[C@@H]6c6ccccc6)cc5)CC4)C3)CC2)ccc1N1CCC(=O)NC1=O]

Step 1: A mixture of 1-(4-bromo-2-methylphenyl)dihydropyrimidine-2,4 (1H,3H)-dione (2 g, 1 Eq. 7.1 mmol), tert-butyl2,8-diazaspiro[4.5]decane-2-carboxylate (2 g, 1.2 Eq, 8.5 mmol), Pd-PEPPSI-lHoptCl (0.69 g, 0.1 Eq, 0.71 mmol) and cesium carbonate (6.9 g, 3 Eq, 21 mmol) in dioxane (30 mL) was stirred at r.t. N₂ was bubbled into the mixture for 5 min. The reaction was heated at 100° C. for 3 h. The mixture was added H₂O (100 mL) and extracted with ethyl acetate (100 mL×2). The combined extracts were dried over anhydrous Na₂SO₄, then filtered and concentrated to dryness in vacuo to give a residue. The residue was subjected to column chromatography over silica gel (petroleum ether/ethyl acetate from 100/0 to 0/100). The pure fractions were collected and concentrated to dryness in vacuo to give tert-butyl 8-(4-(2,4-dioxotetrahydropyrimidin-1 (2H)-yl)-3-methylphenyl)-2,8-diazaspiro[4.5]decane-2-carboxylate (1 g, 30% yield, 85.2% purity) as a white solid. LC-MS (ESI+) m/z: 443.3 (M+H)⁺.

Step 2: A mixture of tert-butyl 8-(4-(2,4-dioxotetrahydropyrimidin-1 (2H)-yl)-3-methylphenyl)-2,8-diazaspiro[4.5]decane-2-carboxylate (1 g, 1 Eq, 2 mmol) in HCl/dioxane (25 mL) was stirred at 25° C. for 16 h. The mixture was concentrated to dryness in vacuo to give 1-(2-methyl-4-(2,8-diazaspiro[4.5]decan-8-yl)phenyl)dihydropyrimidine-2,4 (1H,3H)-dione (0.8 g, 100% yield, 93.3% purity) as a white solid. LC-MS (ESI+) m/z: 343.3 (M+H)⁺

Step 3: A mixture of 1-(4-((1R,2S)-4,4-difluoro-6-hydroxy-2-phenyl-1,2,3,4-tetrahydronaphthalen-1-yl)phenyl)piperidine-4-carbaldehyde (110 mg, 1 Eq, 224 μmol), 1-(2-methyl-4-(2,8-diazaspiro[4.5]decan-8-yl)phenyl)dihydropyrimidine-2,4 (1H,3H)-dione (98.5 mg, 1.2 Eq, 268 μmol), sodium acetate (91.7 mg, 5 Eq, 1.12 mmol) and acetic acid (40.3 mg, 38.6 μL, 3 Eq, 671 μmol) in DCM (3 mL) and MeOH (3 mL) was stirred at 30° C. for 1 h, then sodium triacetoxyborohydride (94.8 mg, 2 Eq, 447 μmol) was added to the mixture and stirred at 30° C. for 16 h. Then the mixture was added H₂O (30 mL) and extracted with ethyl acetate (30 mL×2). The combined organic layers were washed with brine (20 mL), dried over anhydrous Na₂SO₄, filtered and concentrated to dryness in vacuo to give a residue. The residue was purified by prep-HPLC (column: Welch Xtimate C18 150*30 mm*5 um; mobile phase: [water(FA)-ACN]; B % 8%-48%, 9 min) to give 1-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)-2-methylphenyl)dihydropyrimidine-2,4 (1H,3H)-dione (114.7 mg, 63.0% yield, 95.134% purity) as a white solid. LC-MS (ESI+) m/z: 774.4 (M+H)⁺.

LCMS; calc. for C47H₅₃F₂N₅O₃: 773.41, found: [M+H]⁺ 774.4.

HPLC: 95.134% purity at 220 nm.

¹HNMR (400 MHz, DMSO-d₆) δ=10.24 (s, 1H), 8.18 (s, 1H), 7.24-7.16 (m, 3H), 7.10-7.01 (m, 2H), 6.93-6.75 (m, 6H), 6.56 (d, J=8.8 Hz, 2H), 6.13 (d, J=8.8 Hz, 2H), 4.30 (br d, J=2.8 Hz, 1H), 3.71-3.62 (m, 2H), 3.57-3.41 (m, 3H), 3.21-3.04 (m, 4H), 2.77-2.55 (m, 5H), 2.50-2.44 (m, 2H), 2.41 (s, 2H), 2.37-2.26 (m, 3H), 2.11 (s, 3H), 1.74 (br d, J=11.8 Hz, 2H), 1.66-1.49 (m, 7H), 1.20-1.07 (m, 2H)

SFC: retention time, 2.893 min; Area, 100%.

### Example 6. Preparation of (I-290) 1-(4-((S)-7-((1-(4-((1R,2S)-4,4-difluoro-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)dihydropyrimidine-2,4 (1H,3H)-dione

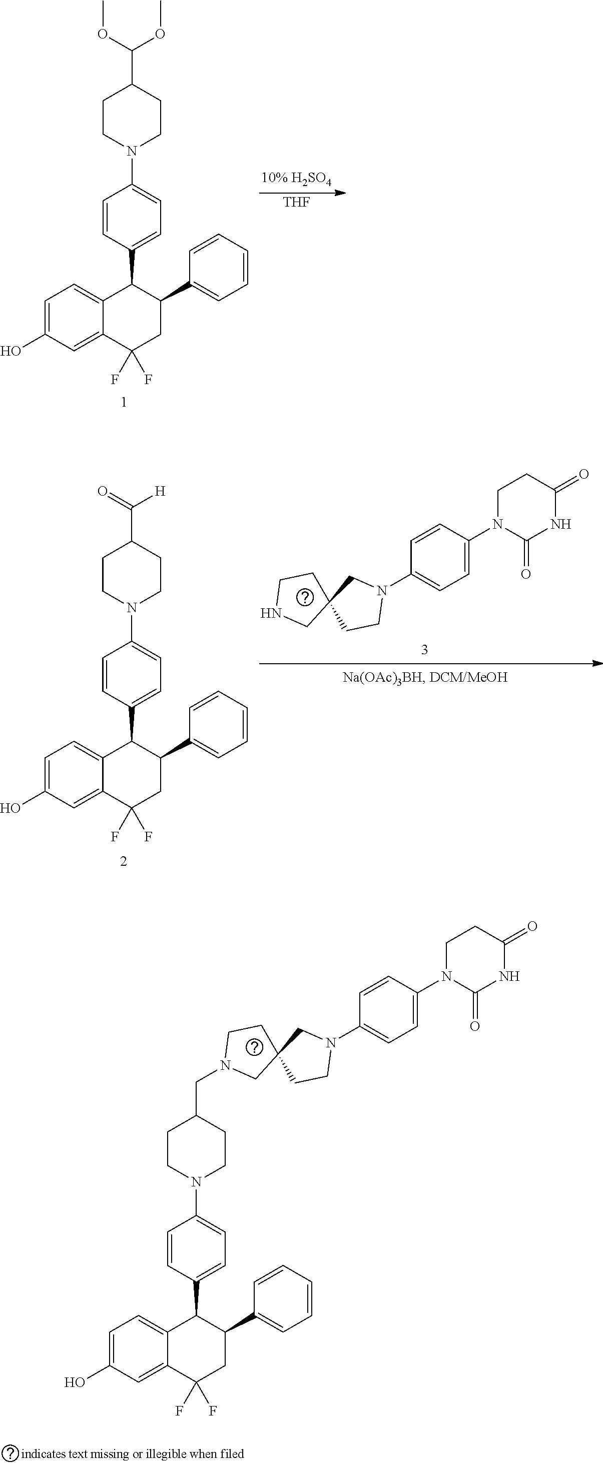 [SMILES: COC(OC)C1CCN(c2ccc([C@@H]3c4ccc(O)cc4C(F)(F)C[C@@H]3c3ccccc3)cc2)CC1 ; O=CC1CCN(c2ccc([C@@H]3c4ccc(O)cc4C(F)(F)C[C@@H]3c3ccccc3)cc2)CC1 ; O=C1CCN(c2ccc(N3CC[C@]4(CCNC4)C3)cc2)C(=O)N1 ; "THF" ; O=C1CCN(c2ccc(N3CC[C@]4(CCN(CC5CCN(c6ccc([C@@H]7c8ccc(O)cc8C(F)(F)C[C@@H]7c7ccccc7)cc6)CC5)C4)C3)cc2)C(=O)N1]

Step 1: To a solution of (5R,6S)-5-(4-(4-(dimethoxymethyl)piperidin-1-yl)phenyl)-8,8-difluoro-6-phenyl-5,6,7,8-tetrahydronaphthalen-2-ol (50 mg, 0.10 mmol, 1.0 eq.) in THF (3 mL) was added H₂SO₄ (0.09 g, 0.05 mL, 9 eq., 0.9 mmol) and water (0.5 mL). The mixture was stirred at 40° C. for 0.5 h. The reaction mixture was quenched by addition of water (10 mL), adjust pH to 8 by NaHCO₃ saturated solution, extracted with EtOAc (20 mL×3). The combined organic layers were washed with brine (10 mL), dried over Na₂SO₄, filtered and concentrated under reduced pressure to give 1-(4-((1R,2S)-4,4-difluoro-6-hydroxy-2-phenyl-1,2,3,4-tetrahydronaphthalen-1-yl)phenyl)piperidine-4-carbaldehyde (40 mg, 89 μmol, 88% yield) as a yellow solid.

LC-MS (ESI⁺) m/z: 448.3 (M+H)⁺.

Step 2: To a solution of 1-(4-((1R,2S)-4,4-difluoro-6-hydroxy-2-phenyl-1,2,3,4-tetrahydronaphthalen e-1-yl)phenyl)piperidine-4-carbaldehyde (40.0 mg, 1 eq., 89.4 μmol) and (S)-1-(4-(2,7-diazaspiro[4.4]nonan-2-yl)phenyl)dihydropyrimidine-2,4 (1H,3H)-dione (33.7 mg, 107 μmol, 1.2 eq.) in DCE (3 mL) and MeOH (3 mL) was added Sodium triacetoxyborohydride (56.8 mg, 268 μmol, 3.0 eq.). The mixture was stirred at 20° C. for 2 hour. LCMS showed the reaction was completed. The crude was purified by prep-HPLC together (Boston Green ODS 150*30 mm*5 um, water(FA)-ACN as a mobile phase, from 18% to 48%, Gradient Time (min): 12, Flow Rate (ml/min): 25) to give 1-(4-((S)-7-((1-(4-((1R,2S)-4,4-difluoro-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)dihydropyrimidine-2,4 (1H,3H)-dione (13.6 mg, 18.2 μmol, 20.4% yield) as a white solid. LC-MS (ESI+) m/z: 746.4 (M+H)⁺.

LCMS: calc. for C₄₅H₄₉F₂N₅O₃: 745.92, found: [M+H]⁺ 746.4.

HPLC: 100.00% purity at 220 nm.

¹H NMR (400 MHZ, METHANOL-d₄) δ: ppm 8.47-8.55 (m, 1H), 7.08-7.20 (m, 6H), 6.78-6.91 (m, 4H), 6.54-6.68 (m, 4H), 6.25 (d, J=8.40 Hz, 2H), 4.25-4.32 (m, 1H), 3.74-3.81 (m, 2H), 3.66-3.73 (m, 1H), 3.52-3.59 (m, 2H), 3.37-3.42 (m, 2H), 3.33-3.36 (m, 2H), 3.27 (s, 2H), 2.98-3.19 (m, 3H), 2.79 (t, J=6.80 Hz, 3H), 2.65-2.73 (m, 1H), 2.55-2.63 (m, 2H), 2.23-2.33 (m, 1H), 1.99-2.16 (m, 4H), 1.81-1.88 (m, 2H), 1.70-1.81 (m, 1H), 1.31-1.39 (m, 2H).

### Example 7. Preparation of (I-98) (S)-3-(5-(4-((1-(4-((1S,2S)-2-cyclohexyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

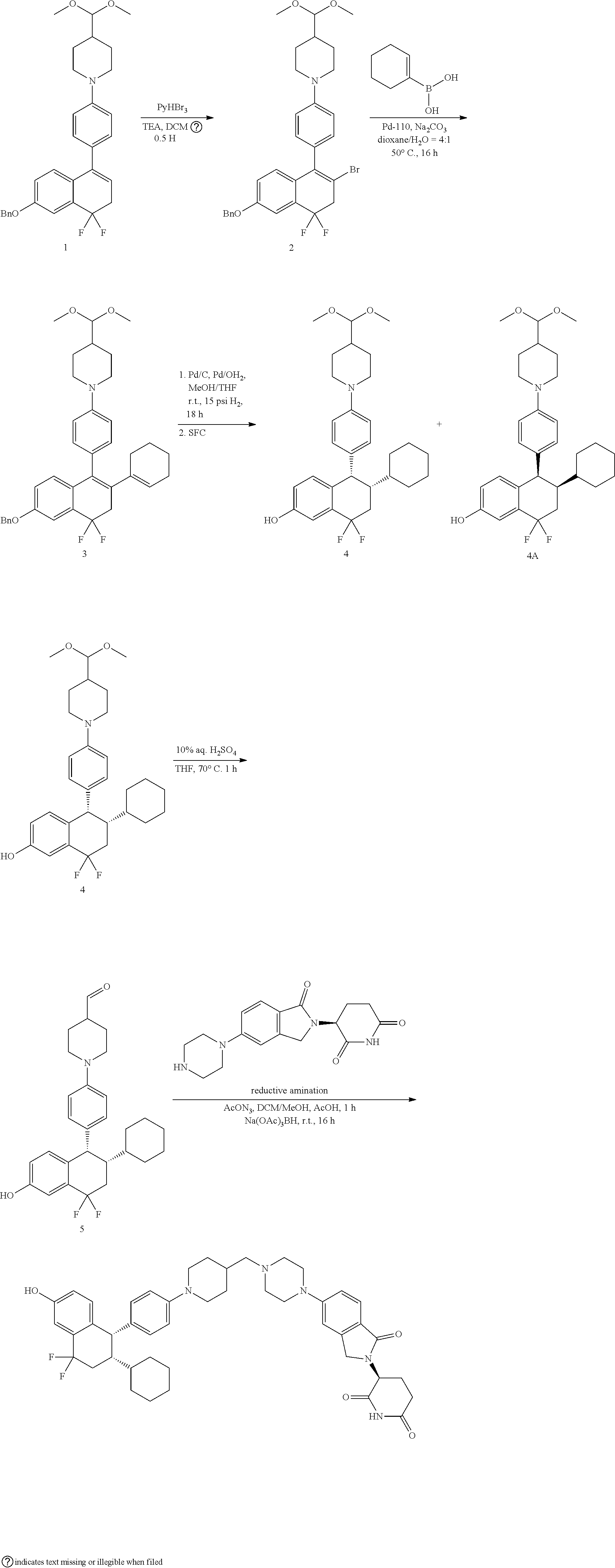

Step 1: To a mixture of 1-(4-(6-(benzyloxy)-4,4-difluoro-3,4-dihydronaphthalen-1-yl)phenyl)-4-(dimethoxymethyl)piperidine (1.2 g, 1 Eq, 2.2 mmol) and TEA (0.33 g, 0.45 mL, 1.5 Eq, 3.2 mmol) in DCM (40 mL) at 0° C., and Pyridinium bromide perbromide (0.76 g, 1.1 Eq, 2.4 mmol) was added to the mixture at 0° C., then the mixture was stirred at 0° C. for 0.5 hour. TLC (petroleum ether:ethyl acetate=5:1, R_{f}=0.5, UV) showed one main new spot was observed. The reaction mixture was quenched by addition H₂O 50 mL, and then diluted with H₂O 50 mL and extracted with DCM (50 mL*2). The organic layer was washed with brine (50 mL) and dried over Na₂SO₄ and concentrated in vacuo to give a yellow solid. The residue was subjected to column chromatography over silica gel (gradient elution: 0˜30% ethyl acetate in petroleum ether). The pure fractions were collected and concentrated to dryness in vacuo to give 1-(4-(6-(benzyloxy)-2-bromo-4,4-difluoro-3,4-dihydronaphthalen-1-yl)phenyl)-4-(dimethoxymethyl)piperidine (1.2 g, 0.95 mmol, 44%, 46.216% purity) as a yellow solid. LC-MS (ESI⁺) m/z: 586.2 (M+H)⁺.

Step 2: A mixture of 1-(4-(6-(benzyloxy)-2-bromo-4,4-difluoro-3,4-dihydronaphthalen-1-yl)phenyl)-4-(dimethoxymethyl)piperidine (1.18 g, 1 Eq, 2.02 mmol), Cyclohexen-1-ylboronicacid (254 mg, 1 Eq, 2.02 mmol), Sodium carbonate (642 mg, 3 Eq, 6.06 mmol) 1,1′-Bis(di-t-butylphosphino)ferrocene palladium dichloride (132 mg, 0.1 Eq. 202 μmol) in 1,4-Dioxane (20 mL) and H₂O (5.0 mL) was degassed and purged with N₂ for 3 times, and then the mixture was stirred at 50° C. for 16 hour under N₂ atmosphere. TLC (petroleum ether:ethyl acetate=5:1, R_{f}=0.5, UV) showed one main new spot was observed. The reaction was quenched with water (50 mL) and extracted with ethyl acetate (150 mL*2). The organic layer was washed with brine (100 mL) and dried over Na₂SO₄ and concentrated in vacuo to give a white solid. The white solid was subjected to column chromatography over silica gel (gradient elution: 0-100% EtOAc). The desired fractions were collected, and concentrated to dryness in vacuo to give 1-(4-(6-(benzyloxy)-2-(cyclohex-1-en-1-yl)-4,4-difluoro-3,4-dihydronaphthalen-1-yl)phenyl)-4-(dimethoxymethyl)piperidine (1.1 g, 0.90 mmol, 45%, 48.181% purity) as a white solid. LC-MS (ESI⁺) m/z: 586.3 (M+H)⁺

Step 3: A mixture of 1-(4-(6-(benzyloxy)-4,4-difluoro-2-phenyl-3,4-dihydronaphthalen-1-yl)phenyl)-4-(dimethoxymethyl)piperidine (1.1 g, 48.181% Wt, 1 Eq, 0.91 mmol), Pd/C (300 mg, 10% Wt, 0.31 Eq, 282 μmol), Pd(OH)₂ (300 mg, 20% Wt, 0.47 Eq, 427 μmol) in MeOH (15 mL) and THF (15 mL) was degassed and purged with H₂ for 3 times, and then the mixture was stirred at 25° C. for 16 hour under H₂ atmosphere (15 psi). The reaction mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um); mobile phase: [0.1% NH₃·H₂O EtOH]; B %: 35%-35%) to give (5S,6S)-6-cyclohexyl-5-(4-(4-(dimethoxymethyl)piperidin-1-yl)phenyl)-8,8-difluoro-5,6,7,8-tetrahydronaphthalen-2-ol (80 mg, 0.16 mmol, 18%) and (5R,6R)-6-cyclohexyl-5-(4-(4-(dimethoxymethyl)piperidin-1-yl)phenyl)-8,8-difluoro-5,6,7,8-tetrahydronaphthalen-2-ol (130 mg, 263 μmol, 29%) as yellow solid. LC-MS (ESI⁺) m/z: 500.2 (M+H−56)⁺.

Step 4: To a solution of (5S, 6S)-6-cyclohexyl-5-(4-(4-(dimethoxymethyl)piperidin-1-yl)phenyl)-8,8-difluoro-5,6,7,8-tetrahydronaphthalen-2-ol (80 mg, 1 Eq, 0.16 mmol) in 10% H₂SO₄ (4 mL) and THF (4 mL) was stirred at 70° C. for 1 hour. LCMS showed 100% desired MS. The reaction was adjust to pH=8 with aq. NaHCO₃ (30 mL). Then the mixture was added water (30 mL) and extracted with ethyl acetate (2×30 mL). The combined organic layers were washed with brine (20 mL), dried over anhydrous Na₂SO₄, filtered and concentrated to dryness in vacuo to give 1-(4-((1S,2S)-2-cyclohexyl-4,4-difluoro-6-hydroxy-1,2,3,4-tetrahydronaphthalen-1-yl)phenyl)piperidine-4-carbaldehyde (80 mg, 0.18 mmol, 100% purity) as a yellow solid. LC-MS (ESI⁺) m/z: 472.3 (M+H₂O)⁺

Step 5: To a solution of 1-(4-((1S,2S)-2-cyclohexyl-4,4-difluoro-6-hydroxy-1,2,3,4-tetrahydronaphthalen-1-yl)phenyl)piperidine-4-carbaldehyde (80 mg, 1 Eq, 0.18 mmol), (R)-3-(1-oxo-5-(piperazin-1-yl)isoindolin-2-yl)piperidine-2,6-dione, TsOH salt (88 mg, 1 Eq, 0.18 mmol) in DCM (3 mL), MeOH (3 mL) was added Sodium acetate (43 mg, 3 Eq, 0.53 mmol) was stirred at 25° C. for 60 mins, Then Na(OAc)₃BH (75 mg, 2 Eq, 0.35 mmol) and acetic acid (32 mg, 30 μL, 3 Eq, 0.53 mmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um, mobile phase: [water(FA)-ACN]; B % 31%-51%, 7 min) to give (S)-3-(5-(4-((1-(4-((1S,2S)-2-cyclohexyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (43.4 mg, 56.1 μmol, 32%, 98.930% Purity) was obtained as a white solid.

LCMS: calc. for C₄₅H₅₃F₂N₅O₄: 765.95, found: [M+H]⁺ 766.2.

HPLC: 98.930% purity at 220 nm.

¹H NMR (400 MHZ, DMSO-d₆) δ=10.95 (s, 1H), 9.63 (br d, J=1.3 Hz, 1H), 8.21-8.18 (m, 0.217H), 7.52 (d, J=8.5 Hz, 1H), 7.09-7.04 (m, 2H), 6.98 (s, 1H), 6.86-6.78 (m, 3H), 6.78-6.73 (m, 2H), 6.87-6.72 (m, 1H), 5.05 (dd, J=5.1, 13.1 Hz, 1H), 4.38-4.29 (m, 1H), 4.26-4.17 (m, 2H), 3.68-3.58 (m, 2H), 3.39 (br s, 1H), 3.31-3.29 (m, 3H), 3.23 (br d, J=6.2 Hz, 1H), 2.95-2.85 (m, 1H), 2.65-2.53 (m, 5H), 2.45-2.34 (m, 2H), 2.29-2.18 (m, 3H), 2.16-2.03 (m, 2H), 2.00-1.92 (m, 2H), 1.83-1.64 (m, 3H), 1.64 (br s, 1H), 1.61-1.51 (m, 2H), 1.30-1.25 (m, 1H), 1.33-1.00 (m, 7H), 0.83-0.70 (m, 1H) SFC: retention time, 2.824 min; Area, 93.589%.

### Example 8. Preparation of (I-99) (S)-3-(5-(4-((1-(4-((1R,2R)-2-cyclohexyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

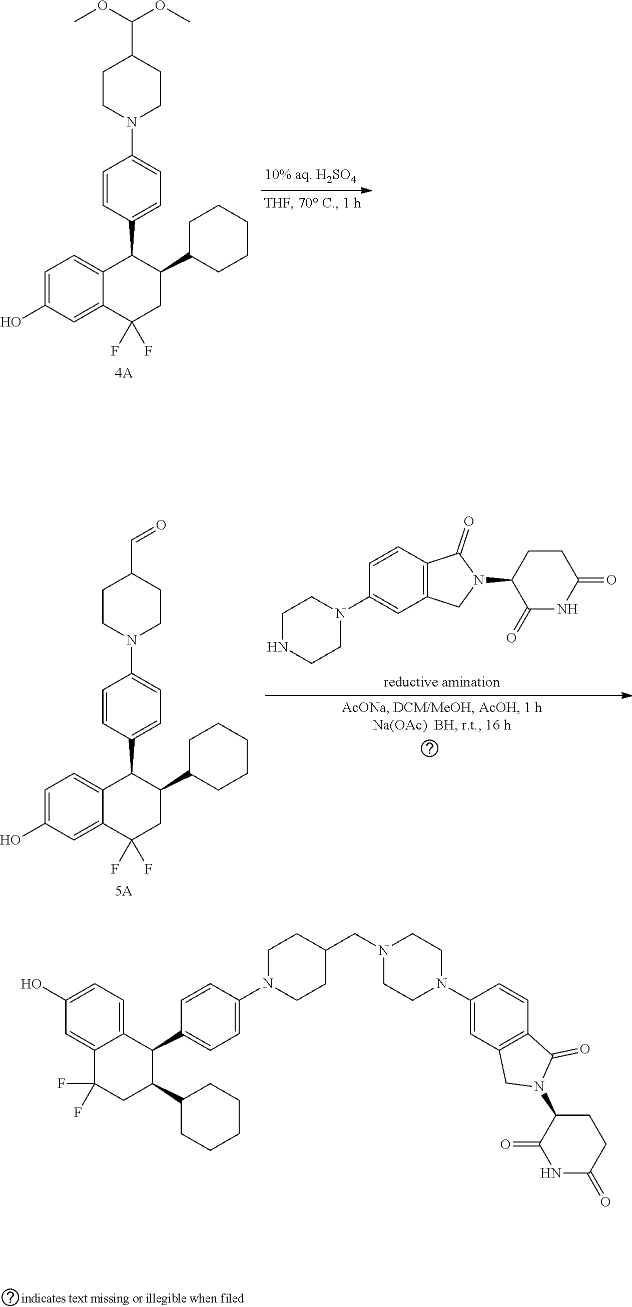 [SMILES: COC(OC)C1CCN(c2ccc([C@@H]3c4ccc(O)cc4C(F)(F)C[C@@H]3C3CCCCC3)cc2)CC1 ; O=CC1CCN(c2ccc([C@@H]3c4ccc(O)cc4C(F)(F)C[C@@H]3C3CCCCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7C7CCCCC7)cc6)CC5)CC4)ccc3C2=O)C(=O)N1]

Step 1: To a solution of (5R, 6R)-6-cyclohexyl-5-(4-(4-(dimethoxymethyl)piperidin-1-yl)phenyl)-8,8-difluoro-5,6,7,8-tetrahydronaphthalen-2-ol (130 mg, 1 Eq, 260 μmol) in 10% H₂SO₄ (4 mL) and THF (4 mL) was stirred at 70° C. for 1 hour. LCMS showed 77.158% desired MS. The reaction was adjust to pH=8 with aq. NaHCO₃ (30 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1R,2R)-2-cyclohexyl-4,4-difluoro-6-hydroxy-1,2,3,4-tetrahydronaphthalen-1-yl)phenyl)piperidine-4-carbaldehyde (140 mg, 269 μmol, 104%, 77.158% purity) as a yellow solid. LC-MS (ESI⁺) m/z: 472.4 (M+H₂O)⁺

Step 2: To a solution of 1-(4-((1R,2R)-2-cyclohexyl-4,4-difluoro-6-hydroxy-1,2,3,4-tetrahydronaphthalen-1-yl)phenyl)piperidine-4-carbaldehyde (140 mg, 1 Eq, 309 μmol), (R)-3-(1-oxo-5-(piperazin-1-yl)isoindolin-2-yl)piperidine-2,6-dione, TsOH salt (154 mg, 1 Eq, 309 μmol) in DCM (3 mL), MeOH (3 mL) was added Sodium acetate (76.0 mg, 3 Eq, 926 μmol) was stirred at 25° C. for 60 mins, Then Na(OAc)₃BH (131 mg, 2 Eq, 617 μmol) and acetic acid (55.6 mg, 53.3 μL, 3 Eq, 926 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um; mobile phase: [water(FA)-ACN]; B % 30%-50%, 7 min) to give (S)-3-(5-(4-((1-(4-((1R,2R)-2-cyclohexyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (62.6 mg. 78.9 μmol, 25.6%, 96.547% Purity) was obtained as a white solid.

LCMS: calc. for C₄₅H₅₃F₂N₅O₄: 765.95, found: [M+H]⁺ 766.2.

HPLC: 96.547% purity at 220 nm.

¹H NMR (400 MHZ, DMSO-d₆) δ=10.95 (s, 1H), 9.71-9.57 (m, 1H), 8.21-8.19 (m, 0.171H), 7.52 (d, J=8.6 Hz, 1H), 7.10-7.04 (m, 2H), 6.98 (s, 1H), 6.78-6.73 (m, 2H), 6.87-6.71 (m, 4H), 5.05 (dd, J=5.1, 13.2 Hz, 1H), 4.37-4.29 (m, 1H), 4.26-4.18 (m, 2H), 3.68-3.57 (m, 2H), 3.41 (br s, 1H), 3.29-3.29 (m, 1H), 3.29-3.25 (m, 1H), 2.96-2.85 (m, 1H), 2.58 (br d, J=13.8 Hz, 4H), 2.53 (br s, 3H), 2.46-2.25 (m, 3H), 2.21 (br d, J=7.3 Hz, 2H), 2.14-1.90 (m, 4H), 1.79 (br d, J=11.6 Hz, 2H), 1.75-1.61 (m, 2H), 1.60-1.52 (m, 2H), 1.30-1.26 (m, 1H), 1.32-0.98 (m, 7H), 0.85-0.70 (m, 1H)

SFC: retention time, 2.922 min; Area, 90.839%.

### Example 9. Preparation of (1-311) (S)-3-(5-(4-((1-(4-((1S,2R)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

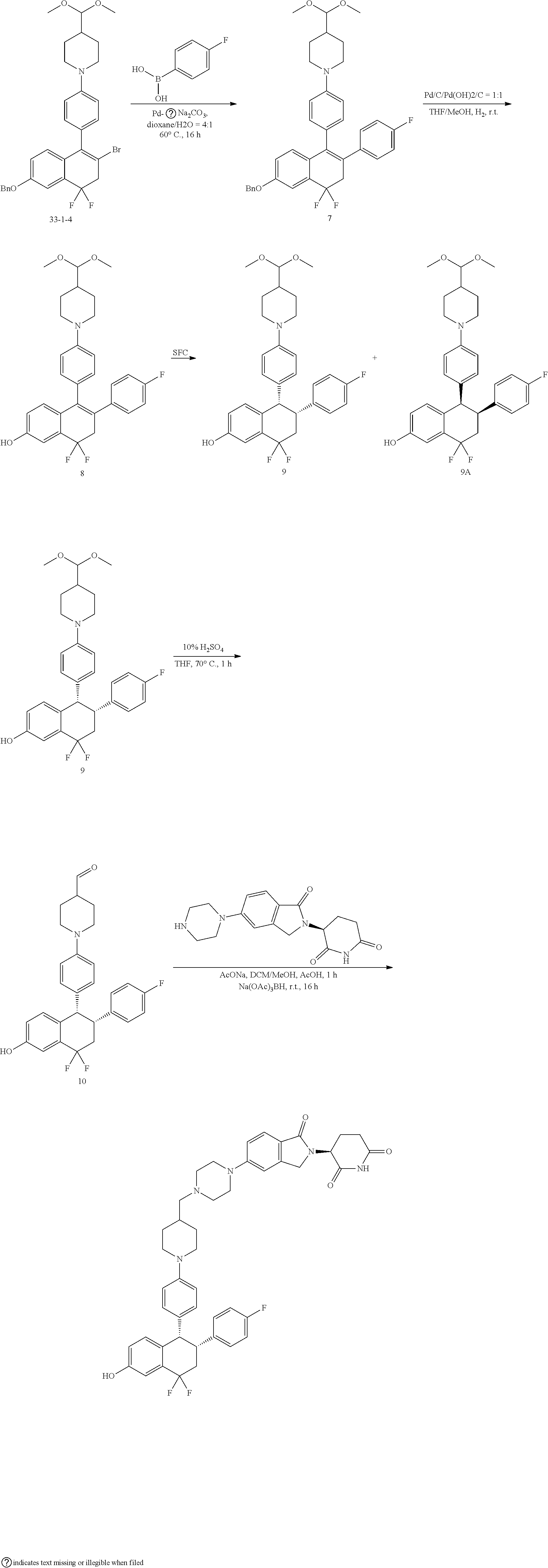

Step 1: 1,1′-Bis(di-t-butylphosphino)ferrocene palladium dichloride (0.10 g, 0.1 Eq, 0.16 mmol) was added to the mixture of 1-(4-(6-(benzyloxy)-2-bromo-4,4-difluoro-3,4-dihydronaphthalen-1-yl)phenyl)-4-(dimethoxymethyl)piperidine (1.0 g, 1 Eq, 1.6 mmol), (4-fluorophenyl) boronic acid (0.33 g, 1.5 Eq, 2.4 mmol) and sodium carbonate (0.50 g, 3 Eq, 4.7 mmol) in dioxane (12 mL) and H₂O (3 mL) at r.t. N₂ was bubbled into the mixture for 5 min. The reaction mixture was heated at 50° C. for 12 h. The mixture was added with H₂O (100 mL) and extracted with EtOAc (100 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-4,4-difluoro-2-(4-fluorophenyl)-3,4-dihydronaphthalen-1-yl)phenyl)-4-(dimethoxymethyl)piperidine (920 mg, 87.484% purity) as a yellow solid. LC-MS (ESI+) m/z: 600.2 (M+H)⁺.

Step 2: A mixture of 1-(4-(6-(benzyloxy)-4,4-difluoro-2-(4-fluorophenyl)-3,4-dihydronaphthalen-1-yl)phenyl)-4-(dimethoxymethyl)piperidine (920 mg, 1 Eq, 1.34 mmol), 10% Pd/C (500 mg, 470 μmol), 20% Pd(OH)₂/C (500 mg, 712 μmol) in MeOH (10 mL) and THE (10 mL) was degassed and purged with H₂ for 3 times, then the mixture was stirred at 25° C. for 16 h under H₂ atmosphere (15 psi). The reaction mixture was filtered and concentrated under reduced pressure to give a residue. The residue was purified by SFC (condition: column: DAICEL CHIRALCEL OJ (250 mm*30 mm, 10 um)); mobile phase: [CO2-MeOH (0.1% NH₃H₂O)]; B %: 35%-35%, min) to give (5S,6R)-5-(4-(4-(dimethoxymethyl)piperidin-1-yl)phenyl)-8,8-difluoro-6-(4-fluorophenyl)-5,6,7,8-tetrahydronaphthalen-2-ol (310 mg, 98.821% purity) and (5R,6S)-5-(4-(4-(dimethoxymethyl)piperidin-1-yl)phenyl)-8,8-difluoro-6-(4-fluorophenyl)-5,6,7,8-tetrahydronaphthalen-2-ol (340 mg, 98.523% purity) as white solids. LC-MS (ESI+) m/z: 512.2 (M+H)⁺.

Step 3: To a solution of (5S,6R)-5-(4-(4-(dimethoxymethyl)piperidin-1-yl)phenyl)-8,8-difluoro-6-(4-fluorophenyl)-5,6,7,8-tetrahydronaphthalen-2-ol (310 mg, 1 Eq, 599 μmol) in THF (4 mL) and 10% H₂SO₄ (4 mL) was stirred at 70° C. for 1 h. The reaction was adjust to pH=8 with aq·NaHCO₃. Then the mixture was added H₂O (50 mL) and extracted with ethyl acetate (50 mL×2). The combined organic layers were washed with brine (50 mL), dried over anhydrous Na₂SO₄, filtered and concentrated to dryness in vacuo to give 1-(4-((1S,2R)-4,4-difluoro-2-(4-fluorophenyl)-6-hydroxy-1,2,3,4-tetrahydronaphthalen-1-yl)phenyl)piperidine-4-carbaldehyde (310 mg, 89.142% purity) as a yellow solid. LC-MS (ESI+) m/z: 466.0 (M+H)⁺.

Step 4: A mixture of 1-(4-((1S,2R)-4,4-difluoro-2-(4-fluorophenyl)-6-hydroxy-1,2,3,4-tetrahydronaphthalen-1-yl)phenyl)piperidine-4-carbaldehyde (310 mg, 1 Eq, 594 μmol), (S)-3-(1-oxo-5-(piperazin-1-yl)isoindolin-2-yl)piperidine-2,6-dione, PhSO₃H salt (386 mg, 1.3 Eq, 772 μmol) and sodium acetate (243 mg, 5 Eq, 2.97 mmol) and acetic acid (107 mg, 3 Eq, 1.78 mmol) in THF (8 mL) and MeOH (8 mL) at 25° C. for 1 h, then sodium triacetoxyborohydride (252 mg, 2 Eq, 1.19 mmol) was added to the mixture and was stirred at 25° C. for 16 h. Then the mixture was added H₂O (50 mL) and extracted with ethyl acetate (50 mL×2). The combined organic layers were washed with brine (50 mL), dried over anhydrous Na₂SO₄, filtered and concentrated to dryness in vacuo to give a residue. The residue was purified by prep-HPLC (column: Xtimate C18 150*40 mm*5 um; mobile phase: [water(FA)-ACN]; B % 13%-53%, 9 min) to give

(S)-3-(5-(4-((1-(4-((1S,2R)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (222.5 mg, 100% purity) as a white solid. LC-MS (ESI+) m/z: 778.1 (M+H)⁺.

LCMS: calc. for C₄₅H₄₆F₃N₅O₄: 777.35, found: [M+H]⁺ 778.1.

HPLC: 100% purity at 220 nm.

¹HNMR (400 MHz, DMSO-d₆) δ=10.96 (s, 1H), 9.76 (br s, 1H), 8.15 (s, 1H), 7.52 (d, J=8.6 Hz, 1H), 7.10-7.00 (m, 5H), 6.97-6.83 (m, 4H), 6.61 (d, J=8.7 Hz, 2H), 6.15 (d, J=8.6 Hz, 2H), 5.05 (dd, J=5.1, 13.3 Hz, 1H), 4.37-4.27 (m, 2H), 4.24-4.17 (m, 1H), 3.75-3.64 (m, 1H), 3.55 (br s, 3H), 3.28 (br s, 7H), 2.96-2.85 (m, 1H), 2.71-2.54 (m, 3H), 2.49-2.47 (m, 1H), 2.43-2.26 (m, 2H), 2.20 (br d, J=7.0 Hz, 2H), 2.02-1.91 (m, 1H), 1.81-1.59 (m, 3H), 1.23-1.08 (m, 2H)

SFC: retention time, 2.504 min; Area, 96.302%.

### Example 10. Preparation of (1-192) (S)-3-(5-(4-((1-(4-((1R,2S)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

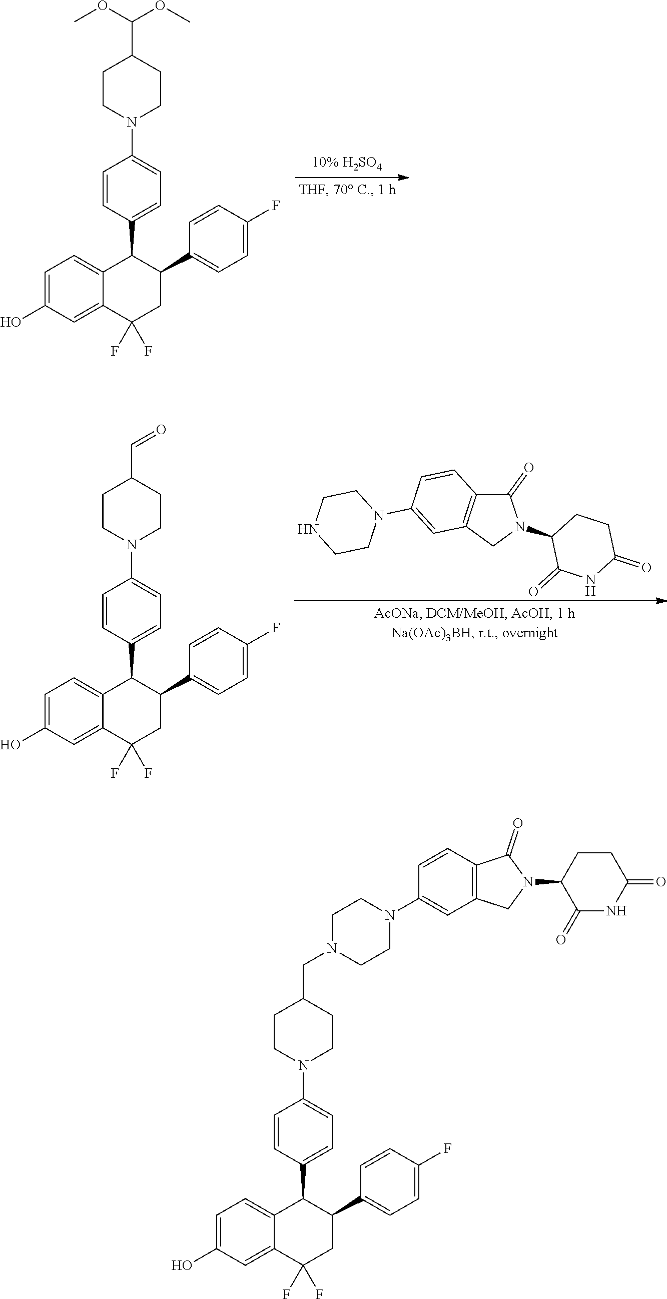 [SMILES: COC(OC)C1CCN(c2ccc([C@@H]3c4ccc(O)cc4C(F)(F)C[C@@H]3c3ccc(F)cc3)cc2)CC1 ; O=CC1CCN(c2ccc([C@@H]3c4ccc(O)cc4C(F)(F)C[C@@H]3c3ccc(F)cc3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1]

Step 1: To a solution of (5R,6S)-5-(4-(4-(dimethoxymethyl)piperidin-1-yl)phenyl)-8,8-difluoro-6-(4-fluorophenyl)-5,6,7,8-tetrahydronaphthalen-2-ol (340 mg, 1 Eq, 655 μmol) in THF (4 mL) and 10% H₂SO₄ (4 mL) was stirred at 70° C. for 1 h. The reaction mixture was quenched by addition NaHCO₃ at 0° C., then added H₂O (30 mL), extracted with EtOAc (50 mL×2). The combined organic layers were washed with brine (30 mL) and dried over Na₂SO₄, filtered and concentrated under reduced pressure to give 1-(4-((1R,2S)-4,4-difluoro-2-(4-fluorophenyl)-6-hydroxy-1,2,3,4-tetrahydronaphthalen-1-yl)phenyl)piperidine-4-carbaldehyde (300 mg, 90.141% purity) as a yellow solid. LC-MS (ESI+) m/z: 466.1 (M+H)⁺.

Step 2: A mixture of 1-(4-((1R,2S)-4,4-difluoro-2-(4-fluorophenyl)-6-hydroxy-1,2,3,4-tetrahydronaphthalen-1-yl)phenyl)piperidine-4-carbaldehyde (300 mg, 1 Eq, 581 μmol), (S)-3-(1-oxo-5-(piperazin-1-yl)isoindolin-2-yl)piperidine-2,6-dione, PhSO₃H salt (378 mg, 1.3 Eq, 755 μmol) and sodium acetate (238 mg, 5 Eq, 2.90 mmol) and acetic acid (105 mg, 3 Eq, 1.74 mmol) in THF (8 mL) and MeOH (8 mL) at 25° C. for 1 h, then sodium triacetoxyborohydride (246 mg, 2 Eq, 1.16 mmol) was added to the mixture and was stirred at 25° C. for 16 h. The mixture was added H₂O (20 mL), extracted with ethyl acetate (20 mL×2). The combined extracts was dried over anhydrous Na₂SO₄, filtered and concentrated to dryness in vacuo to give a residue. The residue was purified by prep-HPLC (column: Welch Xtimate C18 150*30 mm*5 um; mobile phase: [water(FA)-ACN]; B % 13%-53%, 9 min) to give (S)-3-(5-(4-((1-(4-((1R,2S)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (195.8 mg, 99.793% purity) as a white solid. LC-MS (ESI+) m/z: 778.2 (M+H)⁺.

LCMS: calc. for C₄₅H₄₆ F₃N₅O₄: 777.35, found: [M+H]⁺ 778.2.

HPLC: 99.793% purity at 220 nm.

¹HNMR (400 MHz, DMSO-d₆) δ=10.96 (s, 1H), 9.75 (br s, 1H), 8.15 (s, 1H), 7.52 (d, J=8.7 Hz, 1H), 7.11-6.99 (m, 5H), 6.98-6.82 (m, 4H), 6.61 (br d, J=8.7 Hz, 2H), 6.15 (d, J=8.6 Hz, 2H), 5.05 (dd, J=5.1, 13.3 Hz, 1H), 4.37-4.26 (m, 2H), 4.25-4.17 (m, 1H), 3.70 (br d, J=13.4 Hz, 1H), 3.55 (br s, 3H), 3.28 (br s, 7H), 2.97-2.84 (m, 1H), 2.70-2.54 (m, 3H), 2.49-2.47 (m, 1H), 2.42-2.26 (m, 2H), 2.21 (br d, J=6.9 Hz, 2H), 2.02-1.91 (m, 1H), 1.82-1.59 (m, 3H), 1.22-1.07 (m, 2H)

SFC: retention time, 2.620 min; Area, 90.272%.

### Example 11. Preparation of (1-196) (3S)-3-[5-[4-[[1-[4-[(1R,2S)-2-(cyclopentylmethyl)-4,4-difluoro-6-hydroxy-tetralin-1-yl]phenyl]-4-piperidyl]methyl]piperazin-1-yl]-1-oxo-isoindolin-2-yl]piperidine-2,6-dione

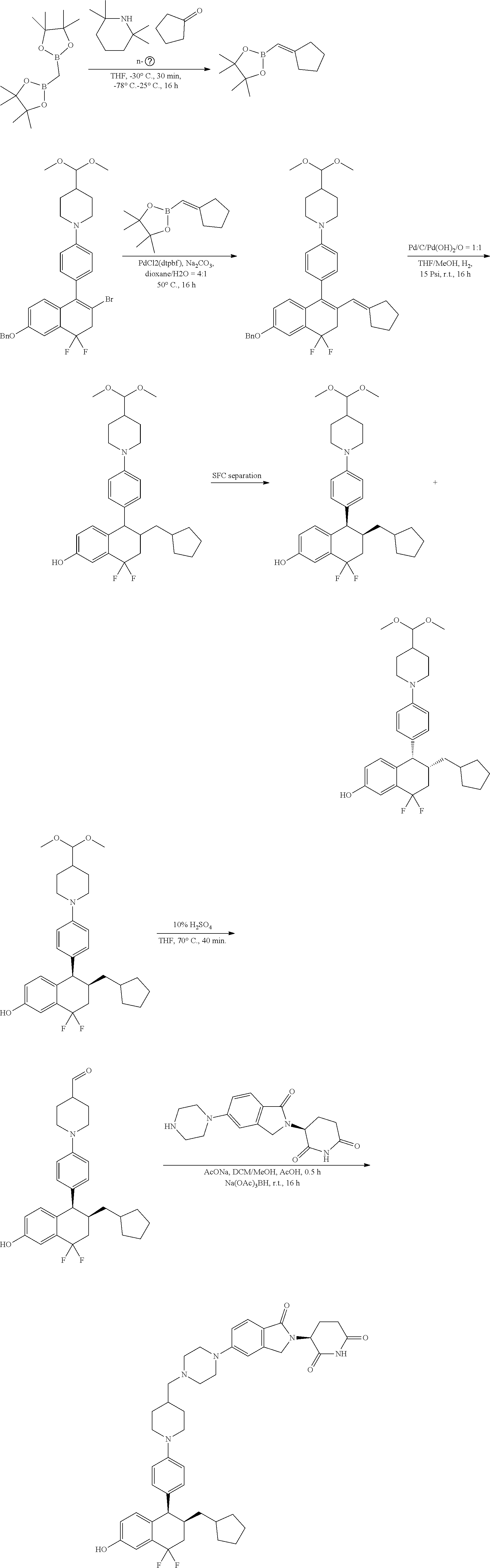

Step 1: A mixture of 2,2,6,6-tetramethyl piperidine (1.2 g, 1.5 mL, 1.2 Eq, 8.9 mmol) was dissolved in THF (20 mL) and cooled to −30° C. under N₂ atmosphere. N-butyllithium (3.6 mL, 2.5 molar, 1.2 Eq, 8.9 mmol) was added dropwise, and the reaction mixture was stirred at the same temperature for 30 min. Next, the reaction was cooled to −78° C., and a solution of bis(4,4,5,5-tetramethyl-1,3,2-dioxaborolan-2-yl) methane (2 g, 1 Eq. 7.46 mmol) in THF (20 mL) was added dropwise. After stirring for 30 min, a solution of cyclopentanone (753 mg, 1.2 Eq, 8.9 mmol) in THF (13 mL) was added dropwise at −78° C. The reaction mixture was allowed to slowly warm up to 25° C., and stirred 16 hour. TLC (PE:EtOAc=2:1, R_{F}=0.4) showed three spots were detected. The reaction was quenched by saturated ammonium chloride solution (30 mL), then stirred for 0.5 h. Water (200 mL) was added then the mixture was extracted with EtOAc (150 mL×3). The organic layers was dried over anhydrous sodium sulfate, filtered and concentrated under reduced pressure to yield (compound B) (cyclopentylidenemethyl)-4, 4, 5, 5-tetramethyl-1, 3, 2-dioxaborolane (1.5 g, 7.2 mmol, 97% yield) as a yellow oil and used without additional purification.

Step 2: A mixture of 1-[4-(6-benzyloxy-2-bromo-4,4-difluoro-3H-naphthalen-1-yl)phenyl]-4-(dimethoxymethyl)piperidine (270 mg, 461.9 μmol, 1 eq), 2-(cyclopentylidenemethyl)-4,4,5,5-tetramethyl-1,3,2-dioxaborolane (288.4 mg, 1.39 mmol, 3 eq), Na₂CO₃ (147 mg, 1.4 mmol, 3 eq), 1,1′-Bis(di-t-butylphosphino)ferrocene palladium dichloride (30.11 mg, 46.19 μmol, 0.1 eq) in dioxane (4 mL) and H₂O (1 mL) was degassed and purged with N₂ for 3 times, and then the mixture was stirred at 50° C. for 16 hr under N₂ atmosphere. The reaction mixture was diluted with 20 mL H₂O and extracted with ethyl acetate (20 mL*2). The combined organic layers were washed with 20 mL brine, dried over Na₂SO₄, filtered and concentrated under reduced pressure to give a residue. The residue was purified by column chromatography (SiO₂, Petroleum ether/ethyl acetate=5/1). Compound 1-[4-[6-benzyloxy-2-(cyclopentylidenemethyl)-4,4-difluoro-3H-naphthalen-1-yl]phenyl]-4-(dimethoxymethyl)piperidine (260 mg, 443.9 μmol, 96.09% yield) was obtained as a yellow solid. LC-MS (ESI+) m/z: 586.3 (M+H)⁺.

Step 3: To a solution of 1-[4-[6-benzyloxy-2-(cyclopentylidenemethyl)-4,4-difluoro-3H-naphthalen-1-yl]phenyl]-4-(dimethoxymethyl)piperidine (400 mg, 683 μmol, 1 eq) in THF (4 mL) and MeOH (4 mL) was added Pd/C (0.2 g, 188 μmol, 10% purity), Pd(OH)₂/C (0.1 g, 163 μmol, 20% purity) under N₂ atmosphere. The suspension was degassed and purged with H₂ for 3 times. The mixture was stirred under H₂ (15 Psi) at 25° C. for 16 hr. The reaction mixture was filtered and concentrated under reduced pressure to give a residue. The residue was purified by prep-HPLC (column: Welch Xtimate C18 150*30 mm*5 um; mobile phase: [water(FA)-ACN]; gradient: 65%-95% B over 7 min) and was further separated by SFC (column: DAICEL CHIRALPAK AD (250 mm*30 mm, 10 um); mobile phase: [CO2-EtOH]; B %: 40%, isocratic elution mode). Compound (1R, 2S)-2-(cyclopentylmethyl)-1-[4-[4-(dimethoxymethyl)-1-piperidyl]phenyl]-4,4-difluoro-tetralin-6-ol (48 mg, 96 μmol, 14.07% yield) was obtained as a yellow solid. Compound (1S, 2R)-2-(cyclopentylmethyl)-1-[4-[4-(dimethoxymethyl)-1-piperidyl]phenyl]-4,4-difluoro-tetralin-6-ol (46 mg, 92.07 μmol, 13.48% yield) was obtained as a white solid. LC-MS (ESI+) m/z: 500.2 (M+H)⁺.

Step 4: To a solution of (1R,2S)-2-(cyclopentylmethyl)-1-[4-[4-(dimethoxymethyl)-1-piperidyl]phenyl]-4,4-difluoro-tetralin-6-ol (48 mg, 96 μmol, 1 eq) in THF (4 mL) was added 10% H₂SO₄ (4 mL). The mixture was stirred at 70° C. for 40 min. The reaction mixture was quenched by addition 20 mL saturated aq. NaHCO₃ solution at 25° C., and then diluted with 5 mL H₂O and extracted with 30 mL ethyl acetate. The combined organic layers were washed with 20 mL brine, dried over Na₂SO₄, filtered and concentrated under reduced pressure to give a residue. The crude product 1-[4-[(1R, 2S)-2-(cyclopentylmethyl)-4,4-difluoro-6-hydroxy-tetralin-1-yl]phenyl]piperidine-4-carbaldehyde (85 mg, 90 μmol, 94% yield, 95% purity) as a yellow oil was used into the next step without further purification. LC-MS (ESI+) m/z: 454.3 (M+H)⁺.

Step 5: To a solution of 1-[4-[(1R,2S)-2-(cyclopentylmethyl)-4,4-difluoro-6-hydroxy-tetralin-1-yl]phenyl]piperidine-4-carbaldehyde (85 mg, 90 μmol, 1.0 eq) and (3S)-3-(1-oxo-5-piperazin-1-yl-isoindolin-2-yl)piperidine-2,6-dione (45 mg, 90 μmol, 1.0 eq, PhSO₃H salt) in DCM (4 mL) and MeOH (4 mL) was added sodium acetate (22 mg, 270 μmol, 3 eq). The mixture was stirred at 30° C. for 0.5 hr. Then the mixture was added NaBH(OAc)₃ (38 mg, 180 μmol, 2 eq) and acetic acid (16 mg, 270 μmol, 15 μL, 3 eq) The mixture was stirred at 30° C. for 16 h. The reaction mixture was diluted with 10 mL H₂O and extracted with 20 mL DCM. The combined organic layers were washed with 10 mL brine, dried over Na₂SO₄, filtered and concentrated under reduced pressure to give a residue. The residue was purified by prep-HPLC (column: Welch Xtimate C18 150*30 mm*5 um; mobile phase: [water(FA)-ACN]; gradient: 27%-57% B over 7 min). Compound (3S)-3-[5-[4-[[1-[4-[(1R,2S)-2-(cyclopentylmethyl)-4,4-difluoro-6-hydroxy-tetralin-1-yl]phenyl]-4-piperidyl]methyl]piperazin-1-yl]-1-oxo-isoindolin-2-yl]piperidine-2,6-dione (26.9 mg, 35 μmol, 39% yield) was obtained as a white solid.

LC-MS (ESI⁺) m/z: 766.5 (M+H)⁺.

HPLC: 98.7%, purity at 220 nm.

SFC: Rt: 3.023 min; Area, 94.214%.

¹H NMR (400 MHZ, DMSO-d₆) δ ppm 10.96 (s, 1H) 9.42-9.92 (m, 1H) 8.16 (s, 0.563H) 7.48-7.56 (m, 1H) 7.03-7.10 (m, 2H) 7.00 (s, 1H) 6.78-6.85 (m, 4H) 6.71 (br d, J=8.58 Hz, 2H) 5.05 (dd, J=13.17, 4.95 Hz, 1H) 4.30-4.37 (m, 1H) 4.16-4.24 (m, 1H) 4.06 (br d, J=1.67 Hz, 1H) 3.61 (br d, J=4.77 Hz, 3H) 3.29 (br s, 8H) 2.86-2.93 (m, 1H) 2.55-2.61 (m, 3H) 2.32-2.40 (m, 1H) 2.18-2.24 (m, 3H) 1.90-2.01 (m, 3H) 1.76-1.83 (m, 2H) 1.71 (br d, J=5.72 Hz, 3H) 1.47-1.60 (m, 4H) 1.15-1.29 (m, 3H) 1.02 (br dd, J=7.03, 3.70 Hz, 2H) 0.82-0.92 (m, 1H)

### Example 12. Preparation of (I-229) (3S)-3-[5-[4-[[1-[4-[(1S,2R)-2-(cyclopentylmethyl)-4,4-difluoro-6-hydroxy-tetralin-1-yl]phenyl]-4-piperidyl]methyl]piperazin-1-yl]-1-oxo-isoindolin-2-yl]piperidine-2,6-dione

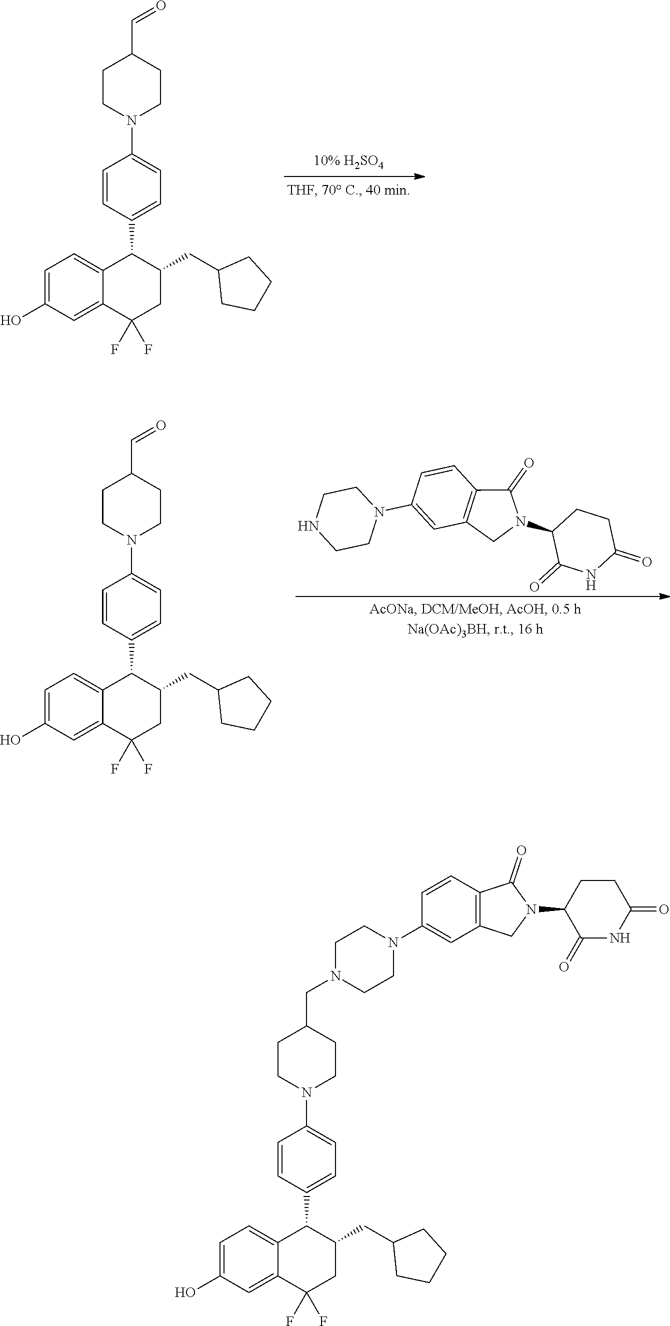 [SMILES: O=CC1CCN(c2ccc([C@H]3c4ccc(O)cc4C(F)(F)C[C@H]3CC3CCCC3)cc2)CC1 ; O=CC1CCN(c2ccc([C@H]3c4ccc(O)cc4C(F)(F)C[C@H]3CC3CCCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7CC7CCCC7)cc6)CC5)CC4)ccc3C2=O)C(=O)N1]

Step 1: To a solution of (1S, 2R)-2-(cyclopentylmethyl)-1-[4-[4-(dimethoxymethyl)-1-piperidyl]phenyl]-4, 4-difluoro-tetralin-6-ol (46.00 mg, 92 μmol, 1 eq) in THF (4 mL) was added 10% H₂SO₄ (4 mL). The mixture was stirred at 70° C. for 40 min. The reaction mixture was quenched by addition 20 mL saturated NaHCO₃ aqueous solution at 25° C., and then diluted with 5 mL H₂O and extracted with 30 mL ethyl acetate. The combined organic layers were washed with brine 20 mL, dried over Na₂SO₄, filtered and concentrated under reduced pressure to give a residue. The crude product 1-[4-[(1S,2R)-2-(cyclopentylmethyl)-4,4-difluoro-6-hydroxy-tetralin-1-yl]phenyl]piperidine-4-carbaldehyde (80 mg, 84.66 μmol, 91.96% yield) was used in the next step without further purification. Compound 1-[4-[(1S,2R)-2-(cyclopentylmethyl)-4,4-difluoro-6-hydroxy-tetralin-1-yl]phenyl]piperidine-4-carbaldehyde (80 mg, 84.66 μmol, 91.96% yield) was obtained as a yellow oil. LC-MS (ESI+) m/z: 454.2 (M+H)⁺.

Step 2: To a solution of 1-[4-[(1S,2R)-2-(cyclopentylmethyl)-4,4-difluoro-6-hydroxy-tetralin-1-yl]phenyl]piperidine-4-carbaldehyde (80 mg, 84.66 μmol, 1 eq) and (3S)-3-(1-oxo-5-piperazin-1-yl-isoindolin-2-yl)piperidine-2,6-dione (42.38 mg, 84.66 μmol, 1 eq, PhSO₃H salt) in DCM (4 mL) and MeOH (4 mL) was added sodium acetate (20.84 mg, 253.99 μmol, 3 eq). The mixture was stirred at 30° C. for 0.5 hr. Then the mixture was added NaBH(OAc)₃ (35 mg, 169 μmol, 2 eq) and acetic acid (15 mg, 254 μmol, 14 μL, 3 eq) The mixture was stirred at r.t. for 16 br finally. The reaction mixture was diluted with 10 mL H₂O and extracted with 20 mL DCM. The combined organic layers were washed with 10 mL brine, dried over Na₂SO₄, filtered and concentrated under reduced pressure to give a residue. The residue was purified by prep-HPLC (column: Welch Xtimate C18 150*30 mm*5 um; mobile phase: [water(FA)-ACN]; gradient: 27%-57% B over 7 min). Compound (3S)-3-[5-[4-[[1-[4-[(1S,2R)-2-(cyclopentylmethyl)-4,4-difluoro-6-hydroxy-tetralin-1-yl]phenyl]-4-piperidyl]methyl]piperazin-1-yl]-1-oxo-isoindolin-2-yl]piperidine-2,6-dione (25.8 mg, 33.68 μmol, 39.79% yield) was obtained as a white solid.

LC-MS (ESI⁺) m/z: 766.5 (M+H)⁺.

HPLC: 98.986%, purity at 220 nm.

SFC: Rt: 2.875 min; Area, 94.205%.

¹H NMR (400 MHZ, DMSO-d₆) δ ppm 10.96 (s, 1H) 9.59-9.74 (m, 1H) 6.17 (s, 0.367H) 7.52 (d, J=8.23 Hz, 1H) 7.03-7.09 (m, 2H) 6.95-7.02 (m, 1H) 6.78-6.85 (m, 4H) 6.71 (br d, J=8.34 Hz, 2H) 5.01-5.09 (m, 1H) 4.29-4.38 (m, 1H) 4.16-4.24 (m, 1H) 4.03-4.09 (m, 1H) 3.58-3.66 (m, 3H) 3.29 (br s, 8H) 2.86-2.94 (m, 1H) 2.56-2.62 (m, 3H) 2.37 (br dd, J=13.47, 4.53 Hz, 1H) 2.18-2.24 (m, 3H) 1.91-2.02 (m, 3H) 1.80 (br d, J=12.87 Hz, 2H) 1.65-1.74 (m, 3H) 1.47-1.60 (m, 4H) 1.16-1.29 (m, 3H) 0.98-1.06 (m, 2H) 0.81-0.93 (m, 1H)

### Example 13. Preparation of (I-234) (S)-3-(5-(4-((1-(4-((1S,2S)-2-cyclopentyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

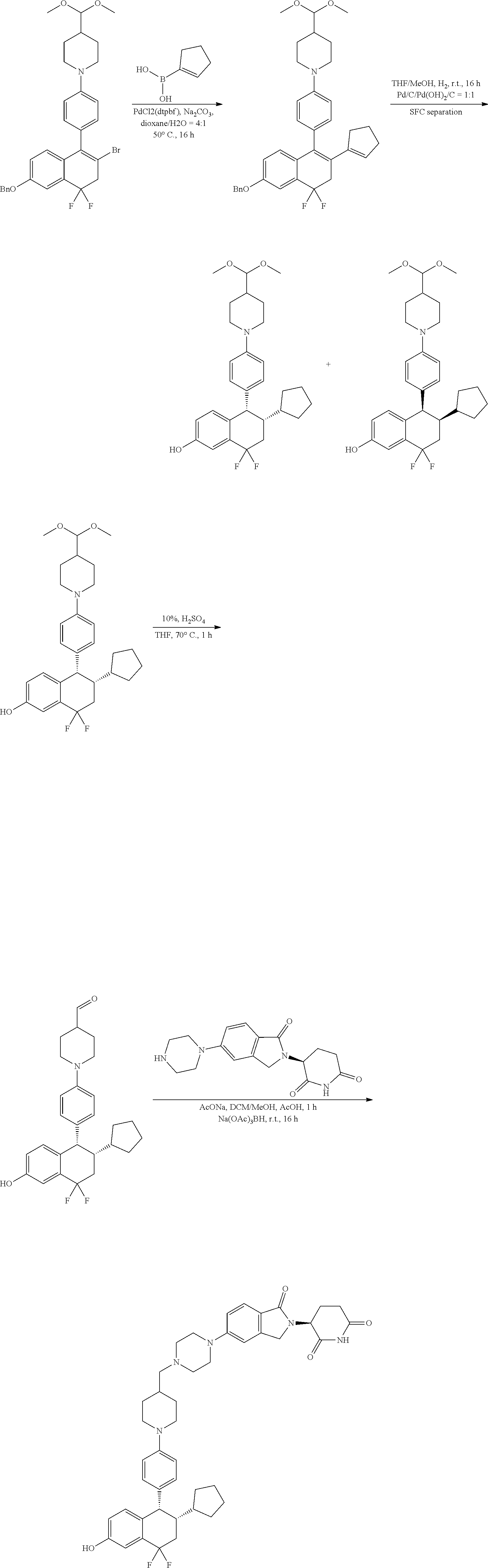

Step 1: 1,1′-Bis(di-t-butylphosphino)ferrocene palladium dichloride (61.5 mg, 0.1 Eq, 94.4 μmol) was added to the mixture of 1-(4-(6-(benzyloxy)-2-bromo-4,4-difluoro-3,4-dihydronaphthalen-1-yl)phenyl)-4-(dimethoxymethyl)piperidine (600 mg, 1 Eq, 944 μmol), cyclopent-1-en-1-ylboronic acid (127 mg, 1.2 Eq, 1.1 mmol) and Na₂CO₃ (300 mg, 3 Eq, 2.8 mmol) in 1,4-dioxane (8 mL) and H₂O (2 mL) at r.t. N₂ was bubbled into the mixture for 5 min. The reaction mixture was heated at 50° C. for 16 hour. Then the reaction was cooled to room temperature. TLC (PE/EtOAc=2/1, R_{f}=0.4) showed a new spot was detected. The mixture was filtered and the filter cake was washed with EtOAc (50 mL). The filtrate was treated with H₂O (150 mL), extracted with EtOAc (150 mL*2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-2-(cyclopent-1-en-1-yl)-4,4-difluoro-3,4-dihydronaphthalen-1-yl)phenyl)-4-(dimethoxymethyl)piperidine (450 mg, 787 μmol, 83.4% yield) as a yellow solid.

LC-MS (ESI⁺) m/z: 571.2 (M+H)⁺.

Step 2: A mixture of 1-(4-(6-(benzyloxy)-2-(cyclopent-1-en-1-yl)-4,4-difluoro-3,4-dihydronaphthalen-1-yl)phenyl)-4-(dimethoxymethyl)piperidine (400 mg, 1 Eq, 700 μmol), Pd/C (200 mg, 10% Wt, 0.269 Eq, 188 μmol), Pd(OH)₂/C (200 mg, 20% Wt, 0.407 Eq, 285 μmol) in MeOH (15 mL) and THF (15 mL) was degassed and purged with H₂ for 3 times, and then the mixture was stirred at 25° C. for 16 hour under H₂ atmosphere (15 psi). The reaction mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um); mobile phase: [0.1% NH₃·H₂O EtOH]; B %: 40%-40%) to give (5S,6S)-6-cyclopentyl-5-(4-(4-(dimethoxymethyl)piperidin-1-yl)phenyl)-8,8-difluoro-5,6,7,8-tetrahydronaphthalen-2-ol (60 mg, 0.11 mmol, 16%, 92.4% purity) and (5R,6R)-6-cyclopentyl-5-(4-(4-(dimethoxymethyl)piperidin-1-yl)phenyl)-8,8-difluoro-5,6,7,8-tetrahydronaphthalen-2-ol (80 mg, 0.15 mmol, 21%, 88.2% purity) as yellow solid. LC-MS (ESI⁺) m/z: 486.2 (M+H)⁺.

Step 3: To a solution of (5S,6S)-6-cyclopentyl-5-(4-(4-(dimethoxymethyl)piperidin-1-yl)phenyl)-8,8-difluoro-5,6,7,8-tetrahydronaphthalen-2-ol (60 mg, 1 Eq, 0.12 mmol) in 10% H₂SO₄ (2 mL) and THF (2 mL) was stirred at 70° C. for 1 hour. TLC (petroleum ether:ethyl acetate=3:1, R_{f}-0.4, UV) showed a new spot was desired. The reaction was adjust to pH=˜8 with aq·NaHCO₃ (20 mL). Then the mixture was added water (30 mL) and extracted with ethyl acetate (30×2 mL). The combined organic layers were washed with brine (30 mL), dried over anhydrous Na₂SO₄, filtered and concentrated to dryness in vacuum to give 1-(4-((1S,2S)-2-cyclopentyl-4,4-difluoro-6-hydroxy-1,2,3,4-tetrahydronaphthalen-1-yl)phenyl)piperidine-4-carbaldehyde (70 mg, 0.16 mmol) as a yellow solid.

Step 4: To a solution of 1-(4-((1S,2S)-2-cyclopentyl-4,4-difluoro-6-hydroxy-1,2,3,4-tetrahydronaphthalen-1-yl)phenyl)piperidine-4-carbaldehyde (70.0 mg, 1 Eq, 159 μmol), (S)-3-(1-oxo-5-(piperazin-1-yl)isoindolin-2-yl)piperidine-2,6-dione, PhSO₃H salt (120 mg, 1.5 Eq, 239 μmol) in DCM (4 mL) and MeOH (4 mL) was added Sodium acetate (39.2 mg, 3 Eq, 478 μmol) was stirred at 25° C. for 60 mins, Then Sodium triacetoxyborohydride (67.5 mg, 2 Eq, 319 μmol) and acetic acid (28.7 mg, 27.5 μL, 3 Eq, 478 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuum to give a yellow oil. The residue was purified by prep-HPLC (column: Welch Xtimate C18 150*30 mm*5 um; mobile phase: [water(FA)-ACN]; B % 23%-53%, 7 min) to give (S)-3-(5-(4-((1-(4-((1S,2S)-2-cyclopentyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (65.4 mg, 86.4 μmol, 54.3%, 99.4% Purity) was obtained as a white solid. LC-MS (ESI⁺) m/z: 752.2 (M+H)⁺.

LCMS: calc. for C₄₁H₅₁F₂N₅O₄: 751.9, found: [M+H]⁺ 752.2 HPLC: 99.4% purity at 220 nm.

¹H NMR (400 MHz, DMSO-d₆) δ=10.96 (s, 1H), 9.65 (br s, 1H), 8.14 (s, 0.474H), 7.53 (d, J=8.7 Hz, 1H), 7.11-7.03 (m, 2H), 6.99 (s, 1H), 6.89-6.72 (m, 6H), 5.06 (dd, J=5.0, 13.2 Hz, 1H), 4.39-4.29 (m, 1H), 4.26-4.17 (m, 1H), 4.12 (br s, 1H), 3.62 (br d, J=8.6 Hz, 2H), 3.30 (br s, 6H), 2.97-2.84 (m, 1H), 2.65-2.55 (m, 5H), 2.40-2.31 (m, 1H), 2.24 (br d, J=6.3 Hz, 3H), 2.18-2.10 (m, 1H), 2.09-2.01 (m, 1H), 1.99-1.86 (m, 2H), 1.80 (br d, J=12.0 Hz, 2H), 1.74-1.64 (m, 1H), 1.63-1.47 (m, 4H), 1.44-1.26 (m, 3H), 1.26-1.13 (m, 2H), 1.09-0.95 (m, 1H)

SFC: retention time, 2.861 min; Area, 94.6%.

### Example 14. Preparation of (1-198) (S)-3-(5-(4-((1-(4-((1R,2R)-2-cyclopentyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

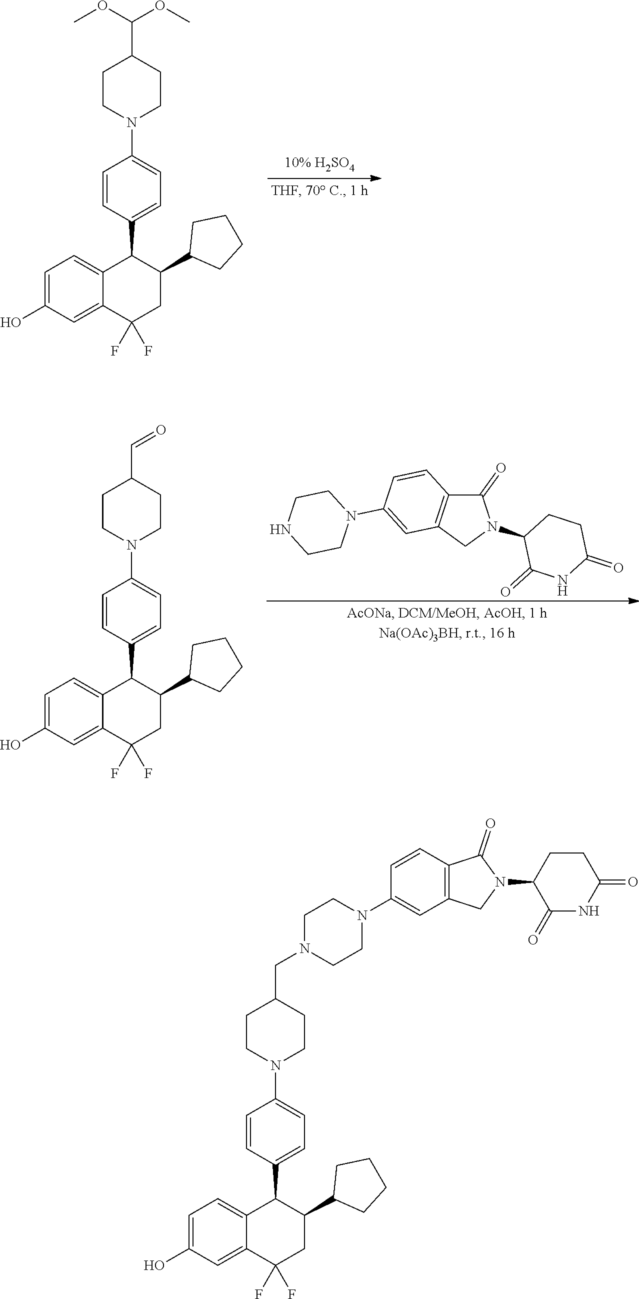 [SMILES: COC(OC)C1CCN(c2ccc([C@@H]3c4ccc(O)cc4C(F)(F)C[C@@H]3C3CCCC3)cc2)CC1 ; O=CC1CCN(c2ccc([C@@H]3c4ccc(O)cc4C(F)(F)C[C@@H]3C3CCCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7C7CCCC7)cc6)CC5)CC4)ccc3C2=O)C(=O)N1]

Step 1: To a solution of (5R,6R)-6-cyclopentyl-5-(4-(4-(dimethoxymethyl)piperidin-1-yl)phenyl)-8,8-difluoro-5,6,7,8-tetrahydronaphthalen-2-ol (80 mg, 1 Eq, 0.16 mmol) in 10% H₂SO₄ (2 mL) and THF (2 mL) was stirred at 70° C. for 1 hour. TLC (petroleum ether:ethyl acetate=3:1, R_{f}=0.4, UV) showed a new spot was desired. The reaction was adjust to pH=˜8 with aq·NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuum to give 1-(4-((1R,2R)-2-cyclopentyl-4,4-difluoro-6-hydroxy-1,2,3,4-tetrahydronaphthalen-1-yl)phenyl)piperidine-4-carbaldehyde (80 mg, 0.18 mmol) as a yellow solid.

Step 2: To a solution of 1-(4-((1R,2R)-2-cyclopentyl-4,4-difluoro-6-hydroxy-1,2,3,4-tetrahydronaphthalen-1-yl)phenyl)piperidine-4-carbaldehyde (80.0 mg, 1 Eq, 182 μmol), (S)-3-(1-oxo-5-(piperazin-1-yl)isoindolin-2-yl)piperidine-2,6-dione, PhSO₃H salt (137 mg, 1.5 Eq, 273 μmol) in DCM (4 mL) and MeOH (4 mL) was added sodium acetate (44.8 mg, 3 Eq, 546 μmol) was stirred at 25° C. for 60 mins, Then sodium triacetoxyborohydride (77.1 mg, 2 Eq, 364 μmol) and acetic acid (32.8 mg, 31.4 μL, 3 Eq, 546 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um; mobile phase: [water(FA)-ACN]; B % 23%-53%, 7 min) to give (S)-3-(5-(4-((1-(4-((1R,2R)-2-cyclopentyl-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (33.6 mg, 44.0 μmol, 24.2%, 98.5% purity) was obtained as a white solid. LC-MS (ESI+) m/z: 752.2 (M+H)⁺.

LCMS: calc. for C₄₁H₅₁F₂N₅O₄: 751.92, found: [M+H]⁺ 752.2 HPLC: 98.5% purity at 220 nm.

¹H NMR (400 MHZ, DMSO-d₆) δ=10.96 (s, 1H), 9.67 (br d, J=5.8 Hz, 1H), 8.17 (s, 0.464H), 7.53 (d, J=8.6 Hz, 1H), 7.10-7.03 (m, 2H), 6.99 (s, 1H), 6.88-6.75 (m, 6H), 5.05 (dd, J=5.0, 13.4 Hz, 1H), 4.39-4.28 (m, 1H), 4.27-4.16 (m, 1H), 4.12 (br d, J=2.4 Hz, 1H), 3.63 (br dd, J=3.1, 8.7 Hz, 3H), 3.29 (br s, 6H), 2.96-2.85 (m, 1H), 2.66-2.55 (m, 4H), 2.40-2.30 (m, 1H), 2.21 (br d, J=7.2 Hz, 3H), 2.17-2.10 (m, 1H), 2.09-2.01 (m, 1H), 1.99-1.87 (m, 2H), 1.80 (br d, J=11.7 Hz, 2H), 1.73-1.64 (m, 1H), 1.62-1.46 (m, 4H), 1.44-1.26 (m, 3H), 1.25-1.11 (m, 2H), 1.08-0.96 (m, 1H)

SFC: retention time, 2.952 min; Area, 95.9%.

### Example 15. Preparation of (I-336) (S)-3-(5-(4-((1-(4-((1S,2R)-4,4-difluoro-6-hydroxy-2-(phenyl-d5)-1,2,3,4-tetrahydronaphthalen-1-yl)phenyl)piperidin-4-yl)methyl)piperazin-1-yl)-1-oxoisoindolin-2-yl)piperidine-2,6-dione

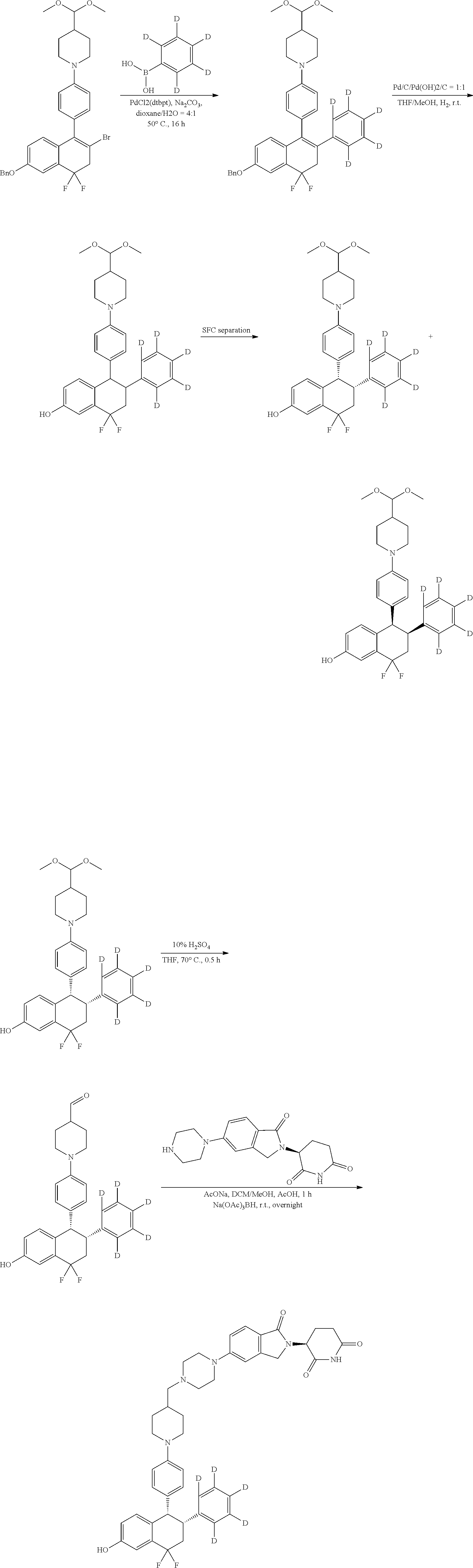

Step 1. 1,1′-Bis(di-t-butylphosphino)ferrocene palladium dichloride (40.8 mg, 0.1 Eq, 62.6 μmol) was added to the mixture of 1-(4-(6-(benzyloxy)-2-bromo-4,4-difluoro-3,4-dihydronaphthalen-1-yl)phenyl)-4-(dimethoxymethyl)piperidine (400 mg, 1 Eq, 626 μmol), B-(Phenyl-2,3,4,5,6-d5) boronic acid (95.3 mg, 1.2 Eq. 751 μmol) and Sodium carbonate (199 mg, 3 Eq, 1.88 mmol) in Dioxane (8 mL) and H₂O (2 mL) at r.t. N₂ was bubbled into the mixture for 5 min. The reaction mixture was heated at 50° C. for 12 h. The mixture was added with H₂O (100 mL) and extracted with EtOAc (100 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-4,4-difluoro-2-(phenyl-d5)-3,4-dihydronaphthalen-1-yl)phenyl)-4-(dimethoxymethyl)piperidine (450 mg, 81.2% Yield) as a yellow solid. LC-MS (ESI+) m/z: 587.0 (M+H)⁺.

Step 2. A mixture of 1-(4-(6-(benzyloxy)-4,4-difluoro-2-(phenyl-d5)-3,4-dihydronaphthalen-1-yl)phenyl)-4-(dimethoxymethyl)piperidine (450 mg, 508 μmol), Pd/C (240 mg, 10% Wt, 226 μmol), Pd(OH)₂/C (240 mg, 20% Wt, 342 μmol) in MeOH (5 mL) and THF (5 mL) was degassed and purged with H₂ for 3 times, and then the mixture was stirred at 25° C. for 16 h with H₂ atmosphere (15 psi). The reaction mixture was filtered and concentrated under reduced pressure to give a residue. The residue was purified by HPLC (condition: column: DAICEL Xtimate C18 150*40 mm*5 um; mobile phase: [water(FA)-ACN]; B %: 30%-70%, 9 min), then was purified by SFC (condition: column: DAICEL CHIRALCEL OJ (250 mm*30 mm, 10 um); mobile phase: [CO2-MeOH (0.1% NH₃H₂O)]; B %: 35%-35%, min) to give (5S,6R)-5-(4-(4-(dimethoxymethyl)piperidin-1-yl)phenyl)-8,8-difluoro-6-(phenyl-d5)-5,6,7,8-tetrahydronaphthalen-2-ol (58 mg, 92.265% purity) (5R,6S)-5-(4-(4-(dimethoxymethyl)piperidin-1-yl)phenyl)-8,8-difluoro-6-(phenyl-d5)-5,6,7,8-tetrahydronaphthalen-2-ol (54 mg, 98.992% purity) as white solids. LC-MS (ESI+) m/z: 499.1 (M+H)⁺.

Step 3. To a solution of (5S,6R)-5-(4-(4-(dimethoxymethyl)piperidin-1-yl)phenyl)-8,8-difluoro-6-(phenyl-d5)-5,6,7,8-tetrahydronaphthalen-2-ol (58 mg, 0.11 mmol) in THF (2 mL) and 10% H₂SO₄ (2 mL) was stirred at 70° C. for 0.5 h. The reaction was adjust to pH=8 with aq. NaHCO₃ at 0° C. Then the mixture was added H₂O (10 mL) and extracted with ethyl acetate (20 mL×2). The combined organic layers were washed with brine (50 mL), dried over anhydrous Na₂SO₄, filtered and concentrated to dryness in vacuo to give 1-(4-((1S,2R)-4,4-difluoro-6-hydroxy-2-(phenyl-d5)-1,2,3,4-tetrahydronaphthalen-1-yl)phenyl)piperidine-4-carbaldehyde (50 mg, 97%, 94.520% Purity) as a yellow solid. LC-MS (ESI+) m/z: 453.2 (M+H)⁺.

Step 4. A mixture of 1-(4-((1S,2R)-4,4-difluoro-6-hydroxy-2-(phenyl-d5)-1,2,3,4-tetrahydronaphthalen-1-yl)phenyl)piperidine-4-carbaldehyde (50 mg, 0.10 mmol), (S)-3-(1-oxo-5-(piperazin-1-yl)isoindolin-2-yl)piperidine-2,6-dione, PhSO₃H salt (63 mg, 1.2 Eq, 0.13 mmol) and sodium acetate (43 mg, 5 Eq, 0.52 mmol) and acetic acid (19 mg, 3 Eq, 0.31 mmol) in THF (3 mL) and MeOH (3 mL) at 25° C. for 1 h, then sodium triacetoxyborohydride (44 mg, 2 Eq, 0.21 mmol) was added to the mixture and was stirred at 25° C. for 16 h. Then the mixture was added H₂O (20 mL) and extracted with EtOAc (20 mL×2). The combined organic layers were washed with brine (20 mL), dried over anhydrous Na₂SO₄, filtered and concentrated to dryness in vacuo to give a residue. The residue was purified by prep-HPLC (column: Welch Xtimate C18 150*30 mm*5 um; mobile phase: [water(FA)-ACN]; B % 15%-55%, 9 min) to give (S)-3-(5-(4-((1-(4-((1S,2R)-4,4-difluoro-6-hydroxy-2-(phenyl-d5)-1,2,3,4-tetrahydronaphthalen-1-yl)phenyl)piperidin-4-yl)methyl)piperazin-1-yl)-1-oxoisoindolin-2-yl)piperidine-2,6-dione (45.6 mg, 57%, 99.842% purity) as a white solid. LC-MS (ESI+) m/z: 765.2 (M+H)⁺.

LCMS: calc. for C₄₅H₄₂D₅F₂N₅O₄: 764.93, found: [M+H]⁺ 765.2.

HPLC: 99.842% purity at 220 nm.

¹HNMR (400 MHZ, DMSO-de) δ=10.96 (s, 1H), 9.74 (br s, 1H), 8.16 (s, 1H), 7.52 (d, J=8.6 Hz, 1H), 7.11-7.03 (m, 3H), 6.91-6.83 (m, 2H), 6.57 (d, J=8.7 Hz, 2H), 6.13 (d, J=8.7 Hz, 2H), 5.05 (dd, J=5.0, 13.2 Hz, 1H), 4.37-4.28 (m, 2H), 4.24-4.16 (m, 1H), 3.67 (br d, J=15.1 Hz, 1H), 3.53 (br d, J=9.4 Hz, 3H), 3.28 (br s, 5H), 2.96-2.85 (m, 1H), 2.75-2.54 (m, 3H), 2.49-2.45 (m, 3H), 2.40-2.30 (m, 2H), 2.19 (br d, J=7.3 Hz, 2H), 2.01-1.91 (m, 1H), 1.75 (br d, J=13.1 Hz, 2H), 1.64 (br s, 1H), 1.22-1.07 (m, 2H) SFC: retention time, 2.620 min; Area, 97.354%.

### Example 16. Preparation of (1-335) (S)-3-(5-(4-((1-(4-((1R,2S)-4,4-difluoro-6-hydroxy-2-(phenyl-d5)-1,2,3,4-tetrahydronaphthalen-1-yl)phenyl)piperidin-4-yl)methyl)piperazin-1-yl)-1-oxoisoindolin-2-yl)piperidine-2,6-dione

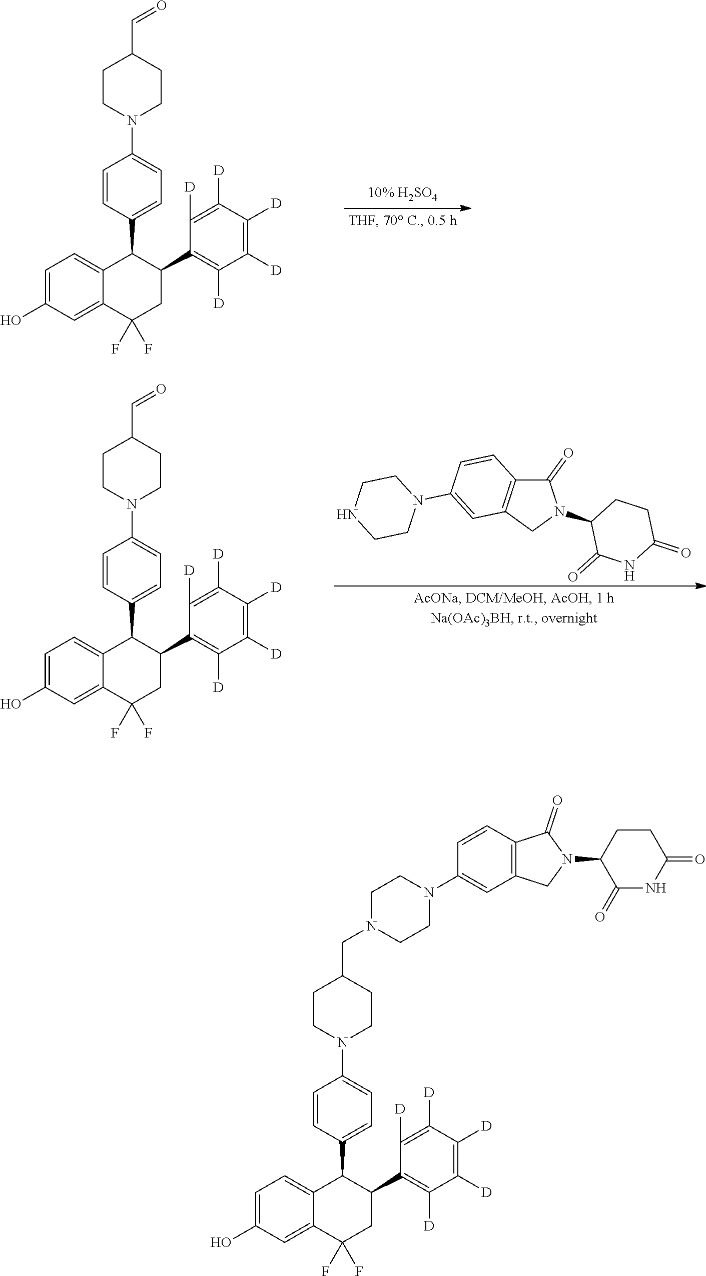

Step 1. To a solution of (5R,6S)-5-(4-(4-(dimethoxymethyl)piperidin-1-yl)phenyl)-8,8-difluoro-6-(phenyl-d5)-5,6,7,8-tetrahydronaphthalen-2-ol (54 mg, 0.11 mmol) in THE (2 mL) and 10% H₂SO₄ (2 mL) was stirred at 70° C. for 0.5 h. The reaction was adjust to pH=8 with aq. NaHCO₃ at 0° C. Then the mixture was added H₂O (10 mL) and extracted with ethyl acetate (20 mL×2). The combined organic layers were washed with brine (50 mL), dried over anhydrous Na₂SO₄, filtered and concentrated to dryness in vacuo to give 1-(4-((1R,2S)-4,4-difluoro-6-hydroxy-2-(phenyl-d5)-1,2,3,4-tetrahydronaphthalen-1-yl)phenyl)piperidine-4-carbaldehyde (50 mg, 96%, 92.771% Purity) as a yellow solid. LC-MS (ESH) m/z: 453.2 (M+H)⁺.

Step 2. A mixture of 1-(4-((1R,2S)-4,4-difluoro-6-hydroxy-2-(phenyl-d5)-1,2,3,4-tetrahydronaphthalen-1-yl)phenyl)piperidine-4-carbaldehyde (50 mg, 0.10 mmol), (S)-3-(1-oxo-5-(piperazin-1-yl)isoindolin-2-yl)piperidine-2,6-dione, PhSO₃H salt (62 mg, 1.2 Eq, 0.12 mmol), sodium acetate (42 mg, 5 Eq, 0.51 mmol) and acetic acid (18 mg, 3 Eq, 0.31 mmol) in THF (3 mL) and MeOH (3 mL) at 25° C. for 1 h, then sodium triacetoxyborohydride (43 mg, 2 Eq, 0.20 mmol) was added to the mixture and was stirred at 25° C. for 16 h. The mixture was added H₂O (20 mL), extracted with EtOAc (20 mL×2). The combined extracts was dried over anhydrous Na₂SO₄, filtered and concentrated to dryness in vacuo to give a residue. The residue was purified by prep-HPLC (column: Welch Xtimate C18 150*30 mm*5 um; mobile phase: [water(FA)-ACN]; B % 15%-55%, 9 min) to give (S)-3-(5-(4-((1-(4-((1R,2S)-4,4-difluoro-6-hydroxy-2-(phenyl-d5)-1,2,3,4-tetrahydronaphthalen-1-yl)phenyl)piperidin-4-yl)methyl)piperazin-1-yl)-1-oxoisoindolin-2-yl)piperidine-2,6-dione (41.4 mg, 53%, 99.557% purity) as a white solid. LC-MS (ESI+) m/z: 765.2 (M+H)⁺.

LCMS: calc. for C₄₅H₄₂D₅F₂N₅O₄: 764.39, found: [M+H]⁺ 765.2.

HPLC: 99.557% purity at 220 nm.

¹HNMR (400 MHZ, DMSO-d₆) δ=10.96 (s, 1H), 9.75 (br s, 1H), 8.17 (s, 1H), 7.52 (d, J=8.7 Hz, 1H), 7.11-7.03 (m, 3H), 6.91-6.83 (m, 2H), 6.57 (d, J=8.7 Hz, 2H), 6.13 (d, J=8.7 Hz, 2H), 5.05 (dd, J=5.1, 13.2 Hz, 1H), 4.37-4.28 (m, 2H), 4.25-4.16 (m, 1H), 3.67 (br d, J=14.9 Hz, 1H), 3.53 (br d, J=9.5 Hz, 3H), 3.28 (br s, 5H), 2.96-2.85 (m, 1H), 2.72-2.55 (m, 3H), 2.49 (br s, 3H), 2.40-2.30 (m, 2H), 2.19 (br d, J=7.0 Hz, 2H), 2.01-1.91 (m, 1H), 1.75 (br d, J=13.4 Hz, 2H), 1.64 (br s, 1H), 1.21-1.07 (m, 2H)

SFC: retention time, 2.739 min; Area, 97.162%.

### Example 17. Preparation of (I-338) (3S)-3-[5-[4-[[1-[5-[(1R,2R)-4,4-difluoro-6-hydroxy-2-phenyl-tetralin-1-yl]-2-pyridyl]-4-piperidyl]methyl]piperazin-1-yl]-1-oxo-isoindolin-2-yl]piperidine-2,6-dione

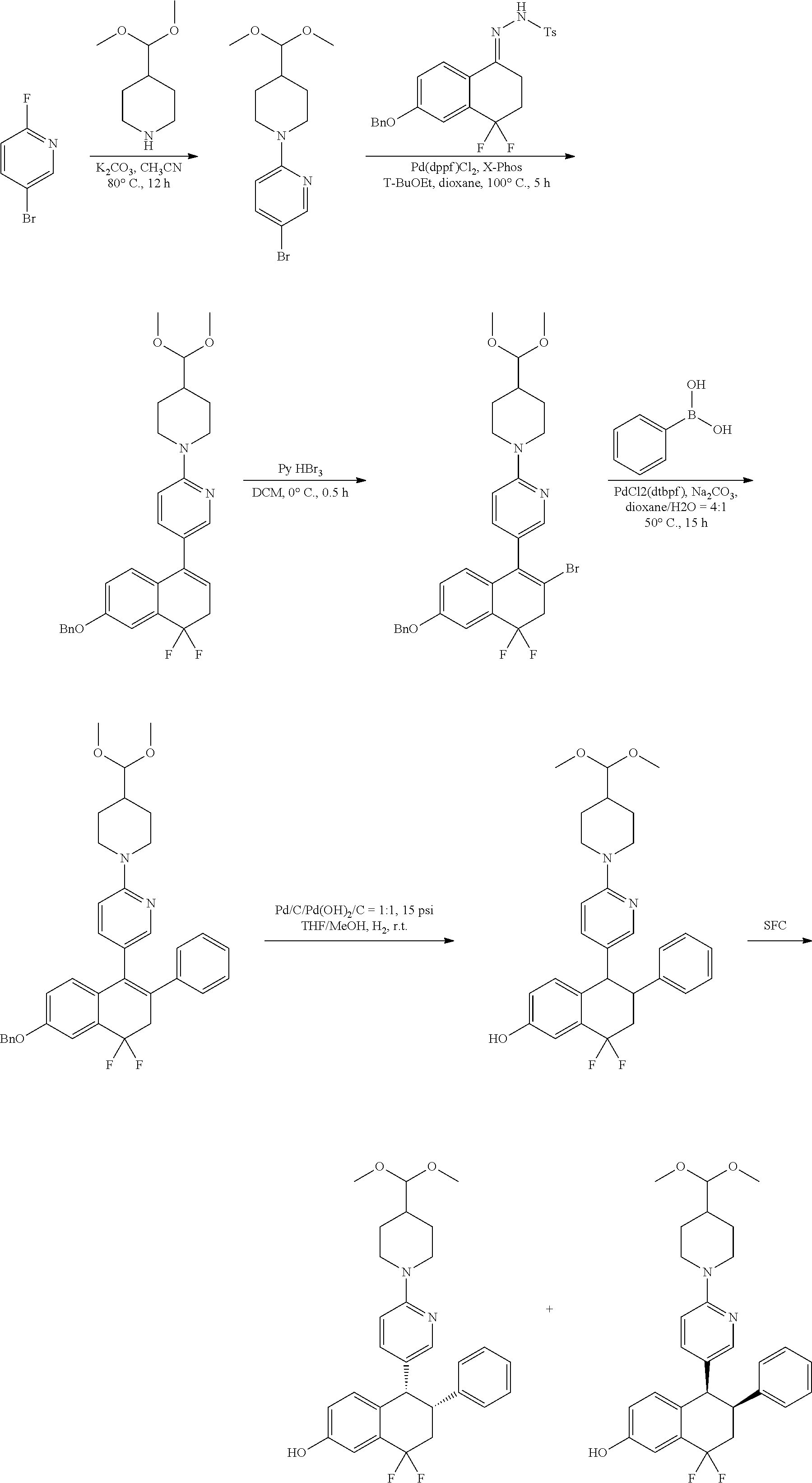

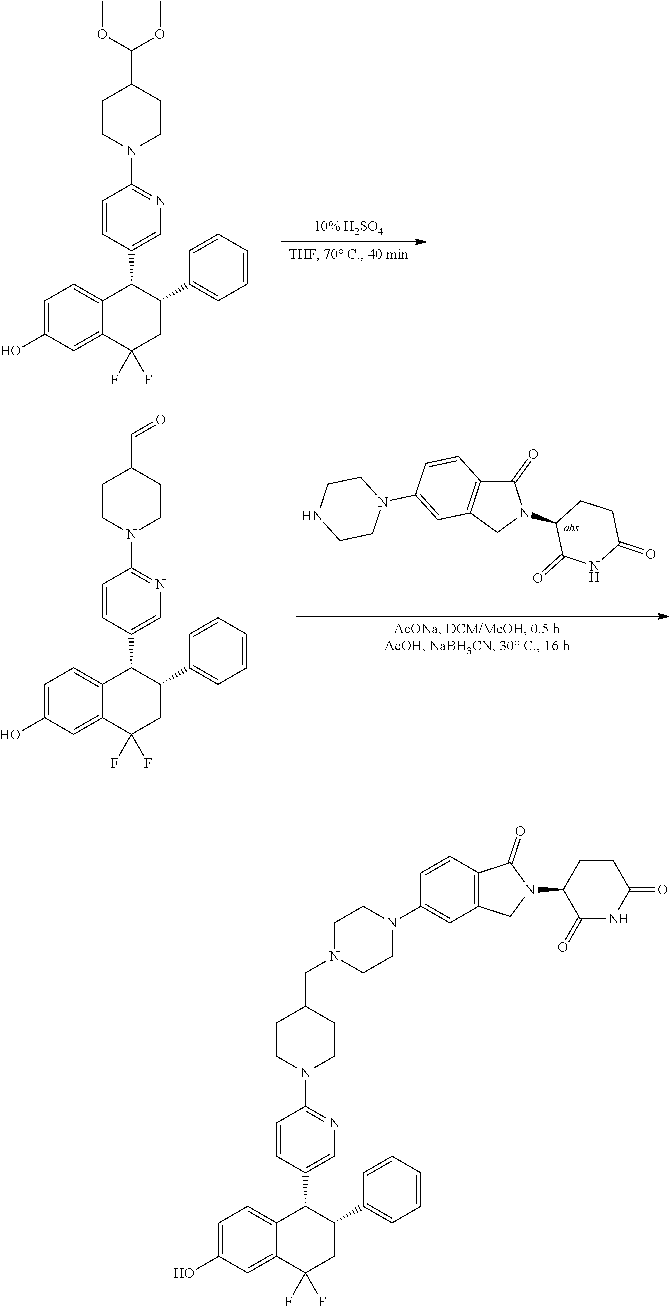

Step 1. To a solution of 5-bromo-2-fluoro-pyridine (5 g, 28.4 mmol, 2.9 mL, 1 eq) and 4˜ (dimethoxymethyl)piperidine (4.5 g, 28.4 mmol, 1 eq) in CH₃CN (20 mL) was added K₂CO₃ (7.8 g, 56.8 mmol, 2 eq). The mixture was stirred at 80° C. for 12 hr. TLC showed (Petroleum ether/Ethyl acetate=10/1, R (0.3) new spot was formed. The reaction mixture was diluted with 50 mL H₂O and extracted with ethyl acetate (40 mL*2). The combined organic layers were washed with brine 40 mL, dried over Na₂SO₄, filtered and concentrated under reduced pressure to give a residue. The residue was purified by column chromatography (SiO₂, Petroleum ether/Ethyl acetate=10/1) Compound 5-bromo-2-[4-(dimethoxymethyl)-1-piperidyl]pyridine (5.1 g, 16.1 mmol, 57% yield) was obtained as a yellow solid. LC-MS (ESI⁺) m/z: 316.7 (M+H)⁺

Step 2. To a solution of 5-bromo-2-[4-(dimethoxymethyl)-1-piperidyl]pyridine (1 g, 3.2 mmol, 1 eq) and N—[(E)-(6-benzyloxy-4,4-difluoro-tetralin-1-ylidene)amino]-4-methyl-benzenesulfonamide (1.4 g, 3.17 mmol, 1 eq) in dioxane (10 mL) was added lithium 2-methylpropan-2-olate (508 mg, 6.3 mmol, 2 eq), Pd(dppf)Cl₂ (259 mg, 317 μmol, 0.1 eq) and X-Phos (756 mg, 1.6 mmol, 0.5 eq). The mixture was stirred at 100° C. for 12 hr. TLC showed (Petroleum ether/Ethyl acetate=5/1, R_{f}=0.3) new spot was formed. The reaction mixture was diluted with 50 mL H₂O and extracted with 50 mL ethyl acetate. The organic layer was washed with brine 50 mL, dried over Na₂SO₄, filtered and concentrated under reduced pressure to give a residue. The residue was purified by column chromatography (SiO₂, Petroleum ether/Ethyl acetate=5/1). Compound 5-(6-benzyloxy-4,4-difluoro-3H-naphthalen-1-yl)-2˜[4-(dimethoxymethyl)-1-piperidyl]pyridine (610 mg, 1.2 mmol, 38% yield) was obtained as a yellow solid. LC-MS (ESI⁺) m/z: 507.1 (M+H)⁺.

Step 3. A mixture of 5-(6-benzyloxy-4,4-difluoro-3H-naphthalen-1-yl)-2-[4˜(dimethoxymethyl)-1-piperidyl]pyridine (610 mg, 1.2 mmol, 1 eq), Py·HBr₃ (385 mg, 1.2 mmol, 1 eq), N,N-diethylethanamine (183 mg, 1.8 mmol, 251 μL, 1.5 eq) in DCM (10 mL) was degassed and purged with N₂ for 3 times, and then the mixture was stirred at 0° C. for 0.5 hr under N₂ atmosphere. TLC showed (Petroleum ether/Ethyl acetate=5/1, R_{f}=0.4) new spot was formed. The residue was purified by column chromatography (SiO₂, Petroleum ether/Ethyl acetate=3/1) Compound 5-(6-benzyloxy-2-bromo-4,4-difluoro-3H-naphthalen-1-yl)-2-[4-(dimethoxymethyl)-1-piperidyl]pyridine (550 mg, 939 μmol, 78% yield) was obtained as a yellow oil. LC-MS (ESI⁺) m/z: 587.3 (M+H)⁺.

Step 4. A mixture of 5-(6-benzyloxy-2-bromo-4,4-difluoro-3H-naphthalen-1-yl)-2-[4-(dimethoxymethyl)-1-piperidyl]pyridine (500 mg, 854 μmol, 1 eq), phenylboronic acid (125 mg, 1 mmol, 1.2 eq), Na₂CO₃ (271 mg, 2.5 mmol, 3 eq), Pd-118 (56 mg, 85 μmol, 0.1 eq) in dioxane (4 mL) and H₂O (1 mL) was degassed and purged with N₂ for 3 times, and then the mixture was stirred at 50° C. for 16 hr under N₂ atmosphere. TLC showed (Petroleum ether/Ethyl acetate=5/1, R_{f}=0.3) new spot was formed. The reaction mixture was diluted with 10 mL H₂O and extracted with ethyl acetate (10 mL*2). The combined organic layers were washed with brine 10 mL (5 mL*2), dried over Na₂SO₄, filtered and concentrated under reduced pressure to give a residue. The residue was purified by column chromatography (SiO₂, Petroleum ether/Ethyl acetate=3/1). Compound 5-(6-benzyloxy-4,4-difluoro-2-phenyl-3H-naphthalen-1-yl)-2-[4-(dimethoxymethyl)-1-piperidyl]pyridine (500 mg, 772 μmol, 90% yield, 90% purity) was obtained as a yellow solid. LC-MS (ESI⁺) m/z: 583.2 (M+H)⁺.

Step 5. To a solution of 5-(6-benzyloxy-4,4-difluoro-2-phenyl-3H-naphthalen-1-yl)-2-[4-(dimethoxymethyl)-1-piperidyl]pyridine (500 mg, 858 μmol, 1 eq) in THF (4 mL) and MeOH (4 mL) was added Pd/C (10%, 0.25 g) and Pd(OH)₂/C (20%, 0.25 g) under N₂ atmosphere. The suspension was degassed and purged with H₂ for 3 times. The mixture was stirred under H₂ (15 Psi) at 25° C. for 12 hr. TLC showed (Petroleum ether/Ethyl acetate=5/1, R (0.3) new spot was formed. The reaction mixture was filtered and concentrated under reduced pressure to give a residue. The residue was purified by prep-HPLC (column: Phenomenex C18 80*40 mm*3 um; mobile phase: [water (NH₃H₂O+NH₄HCO₃)-ACN]; gradient: 62%-92% B over 7 min). Then the residue was further separated by SFC (column: DAICEL CHIRALPAK AD (250 mm*30 mm, 10 um); mobile phase: [CO₂-i-PrOH (0.1% NH₃H₂O)]; B %: 30% %, isocratic elution mode). Compound (1R,2R)-1-[6-[4-(dimethoxymethyl)-1-piperidyl]-3-pyridyl]-4,4-difluoro-2-phenyl-tetralin-6-ol (70 mg, 141 μmol, 16.5% yield) was obtained as a white solid. Compound (1S,2S)-1-[6-[4-(dimethoxymethyl)-1-piperidyl]-3-pyridyl]-4,4-difluoro-2-phenyl-tetralin-6-ol (60 mg, 121 μmol, 14% yield) was obtained as a white solid. LC-MS (ESI+) m/z: 495.1 (M+H)⁺.

Step 6. To a solution of (1R,2R)-1-[6-[4-(dimethoxymethyl)-1-piperidyl]-3-pyridyl]-4,4-difluoro-2-phenyl-tetralin-6-ol (70 mg, 141.54 μmol, 1 eq) in THF (4 mL) was added H₂SO₄ (4 mL, 10%). The mixture was stirred at 70° C. for 40 min. LCMS showed 88% desired MS. The reaction mixture was quenched by Saturated NaHCO₃ aqueous solution addition 20 mL at 25° C., and then diluted with 20 mL H₂O and extracted with 30 mL ethyl acetate. The combined organic layers were washed with 20 mL brine, dried over Na₂SO₄, filtered and concentrated under reduced pressure to give a residue. The crude product 1˜[5-[(1R,2R)-4,4-difluoro-6-hydroxy-2-phenyl-tetralin-1-yl]-2-pyridyl]piperidine-4-carbaldehyde (68 mg, 130.39 μmol, 92% yield) was used into the next step without further purification. Compound 1-[5-[(1R,2R)-4,4-difluoro-6-hydroxy-2-phenyl-tetralin-1-yl]-2-pyridyl]piperidine-4-carbaldehyde (68 mg, 130 μmol, 92% yield) was obtained as a yellow oil. LC-MS (ESI⁺) m/z: 449.2 (M+H)⁺.

Step 7. To a solution of 1-[5-[(1R,2R)-4,4-difluoro-6-hydroxy-2-phenyl-tetralin-1-yl]-2-pyridyl]piperidine-4-carbaldehyde (68 mg, 130.39 μmol, 1 eq) and (3S)-3-(1-oxo-5-piperazin-1-yl-isoindolin-2-yl)piperidine-2,6-dione (65 mg, 130 μmol, 1 eq, PhSO₃H salt) in DCM (4 mL) and MeOH (4 mL) was added sodium acetate (32 mg, 391 μmol, 3 eq). The mixture was stirred at 30° C. for 0.5 hr. Then the mixture was added NaBH(OAc) 3 (55 mg, 260 μmol, 2 eq) and acetic acid (23 mg, 391 μmol, 22 μL, 3 eq) The mixture was stirred at 30° C. for 16 hr finally. The reaction mixture was diluted with 20 mL H₂O and extracted with 20 mL DCM. The combined organic layers were washed with 20 mL brine, dried over Na₂SO₄, filtered and concentrated under reduced pressure to give a residue. The residue was purified by prep-HPLC (column: Welch Xtimate C18 150*30 mm*5 um; mobile phase: [water(FA)-ACN]; gradient: 12%-42% B over 7 min) Compound (3S)-3-[5-[4-[[1-[5-[(1R,2R)-4,4-difluoro-6-hydroxy-2-phenyl-tetralin-1-yl]-2-pyridyl]-4-piperidyl]methyl]piperazin-1-yl]-1-oxo-isoindolin-2-yl]piperidine-2,6-dione (17 mg, 22.34 μmol, 17.14% yield) was obtained as a white solid. LC-MS (ESI⁺) m/z: 761.4 (M+H)⁺.

HPLC: 99.157% purity at 220 nm.

SFC: Rt: 2.649 min; Area, 94.975%.

¹H NMR (400 MHZ, DMSO-d₆) δ ppm 10.96 (s, 1H) 9.76-9.82 (m, 1H) 8.14 (s, 0.424H) 7.52 (d, J=8.46 Hz, 1H) 7.20-7.27 (m, 3H) 7.04-7.11 (m, 3H) 6.97-7.01 (m, 3H) 6.85-6.93 (m, 2H) 6.47-6.55 (m, 1H) 6.37-6.45 (m, 1H) 5.05 (dd, J=13.23, 5.25 Hz, 1H) 4.29-4.37 (m, 2H) 4.16-4.24 (m, 1H) 4.06-4.14 (m, 2H) 3.65-3.74 (m, 1H) 3.29 (br d, J=1.43 Hz, 8H) 2.84-2.96 (m, 1H) 2.60-2.69 (m, 4H) 2.32-2.39 (m, 2H) 2.13-2.26 (m, 2H) 1.91-2.00 (m, 1H) 1.68-1.80 (m, 3H) 0.95-1.09 (m, 2H)

### Example 18. Preparation of (I-202) (3S)-3-[5-[4-[[1-[S-[(1S,2S)-4,4-difluoro-6-hydroxy-2-phenyl-tetralin-1-yl]-2-pyridyl]-4-piperidyl]methyl]piperazin-1-yl]-1-oxo-isoindolin-2-yl]piperidine-2,6-dione

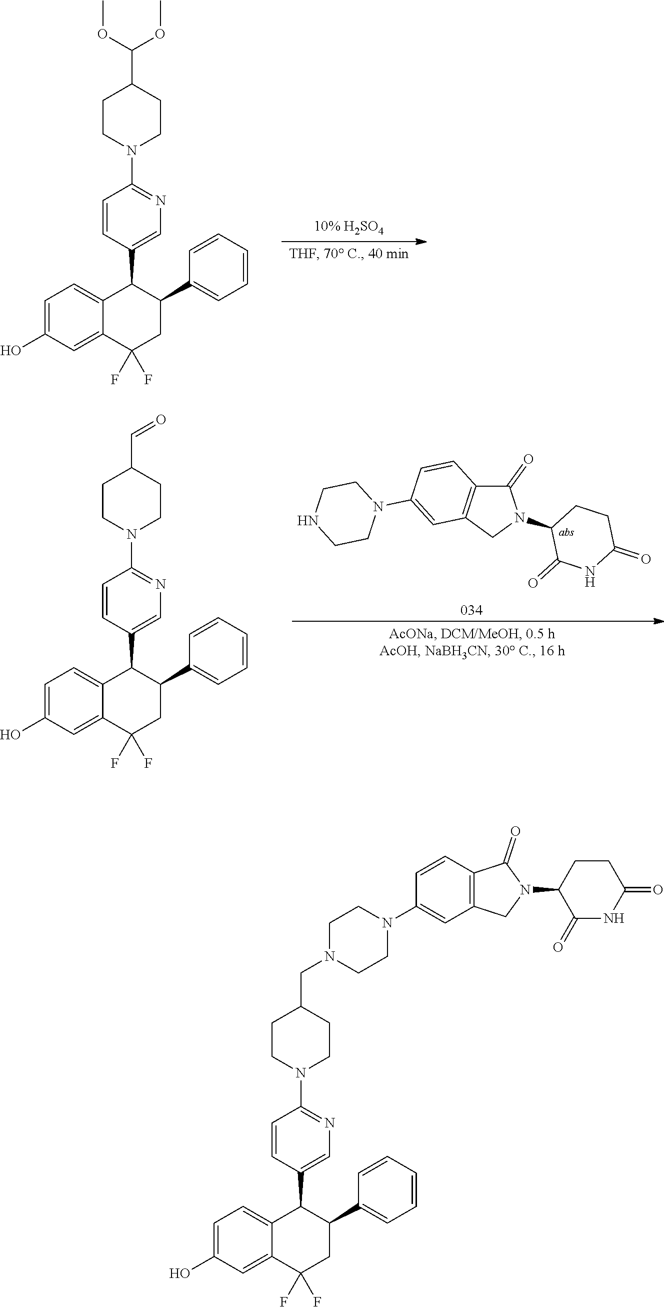

Step 1. To a solution of (1S,2S)-1-[6-[4-(dimethoxymethyl)-1-piperidyl]-3-pyridyl]-4,4-difluoro-2-phenyl-tetralin-6-ol (60 mg, 121 μmol, 1 eq) in THF (4 mL) was added H₂SO₄ (4 mL, 10%). The mixture was stirred at 70° C. for 40 min. LCMS showed 88% desired MS. The reaction mixture was quenched by Saturated NaHCO₃ aqueous solution addition 20 mL at 25° C., and then diluted with 20 mL H₂O and extracted with 30 mL ethyl acetate. The combined organic layers were washed with 20 mL brine, dried over Na₂SO₄, filtered and concentrated under reduced pressure to give a residue. The crude product 1-[5-[(1S,2S)-4,4-difluoro-6-hydroxy-2-phenyl-tetralin-1-yl]-2-pyridyl]piperidine-4-carbaldehyde (58 mg, 116 μmol, 95% yield) was used into the next step without further purification. Compound 1-[5-[(1S,2S)-4,4-difluoro-6-hydroxy-2-phenyl-tetralin-1-yl]-2-pyridyl]piperidine-4-carbaldehyde (58 mg, 116 μmol, 95% yield) was obtained as a yellow oil. LC-MS (ESI⁺) m/z: 449.2 (M+H)⁺.

Step 2. To a solution of 1-[5-[(1S,2S)-4,4-difluoro-6-hydroxy-2-phenyl-tetralin-1-yl]-2-pyridyl]piperidine-4-carbaldehyde (58 mg, 116 μmol, 1 eq) and (3S)-3-(1-oxo-5-piperazin-1-yl-isoindolin-2-yl)piperidine-2,6-dione (58 mg, 116 μmol, 1 eq, PhSO₃H salt) in DCM (4 mL) and MeOH (4 mL) was added sodium acetate (28 mg, 349 μmol, 3 eq). The mixture was stirred at 30° C. for 0.5 hr. Then the mixture was added NaBH(OAc) 3 (49 mg, 232 μmol, 2 eq) and acetic acid (21 mg, 349 μmol, 20 μL, 3 eq) The mixture was stirred at 30° C. for 16 hr finally. The reaction mixture was diluted with 20 mL H₂O and extracted with 20 mL DCM. The combined organic layers were washed with 20 mL brine, dried over Na₂SO₄, filtered and concentrated under reduced pressure to give a residue. The residue was purified by prep-HPLC (column; Welch Xtimate C18 150*30 mm*5 um; mobile phase: [water(FA)-ACN]; gradient: 12%-42% B over 7 min) Compound (3S)-3-[5-[4-[[1-[5-[(1S,2S)-4,4-difluoro-6-hydroxy-2-phenyl-tetralin-1-yl]-2-pyridyl]-4-piperidyl]methyl]piperazin-1-yl]-1-oxo-isoindolin-2-yl]piperidine-2,6-dione (28.8 mg, 37 μmol, 32% yield) was obtained as a white solid. LC-MS (ESI⁺) m/z: 761.4 (M+H)⁺.

HPLC: 99.115% purity at 220 nm.

SFC: Rt: 2.778 min; Area, 95.682%.

¹H NMR (400 MHz, DMSO-d₆) δ ppm 10.96 (s, 1H) 9.79 (s, 1H) 8.14 (s, 0.376H) 7.52 (d, J=8.46 Hz, 1H) 7.21-7.28 (m, 3H) 7.05-7.11 (m, 3H) 6.96-7.00 (m, 3H) 6.85-6.92 (m, 2H) 6.47-6.53 (m, 1H) 6.38-6.45 (m, 1H) 5.05 (dd, J=13.29, 5.07 Hz, 1H) 4.29-4.37 (m, 2H) 4.17-4.24 (m, 1H) 4.05-4.13 (m, 2H) 3.66-3.74 (m, 1H) 3.25-3.34 (m, 8H) 2.84-2.96 (m, 1H) 2.60-2.69 (m, 4H) 2.32-2.39 (m, 2H) 2.13-2.29 (m, 2H) 1.91-2.00 (m, 1H) 1.67-1.80 (m, 3H) 0.96-1.08 (m, 2H)

### Example 19. Preparation of (1-220) (S)-3-(5-(4-((1-(4-((1S,2R)-4,4-difluoro-6-hydroxy-2-isobutyl-1,2,3,4-tetrahydronaphthalen-1-yl)phenyl)piperidin-4-yl)methyl)piperazin-1-yl)-1-oxoisoindolin-2-yl)piperidine-2,6-dione

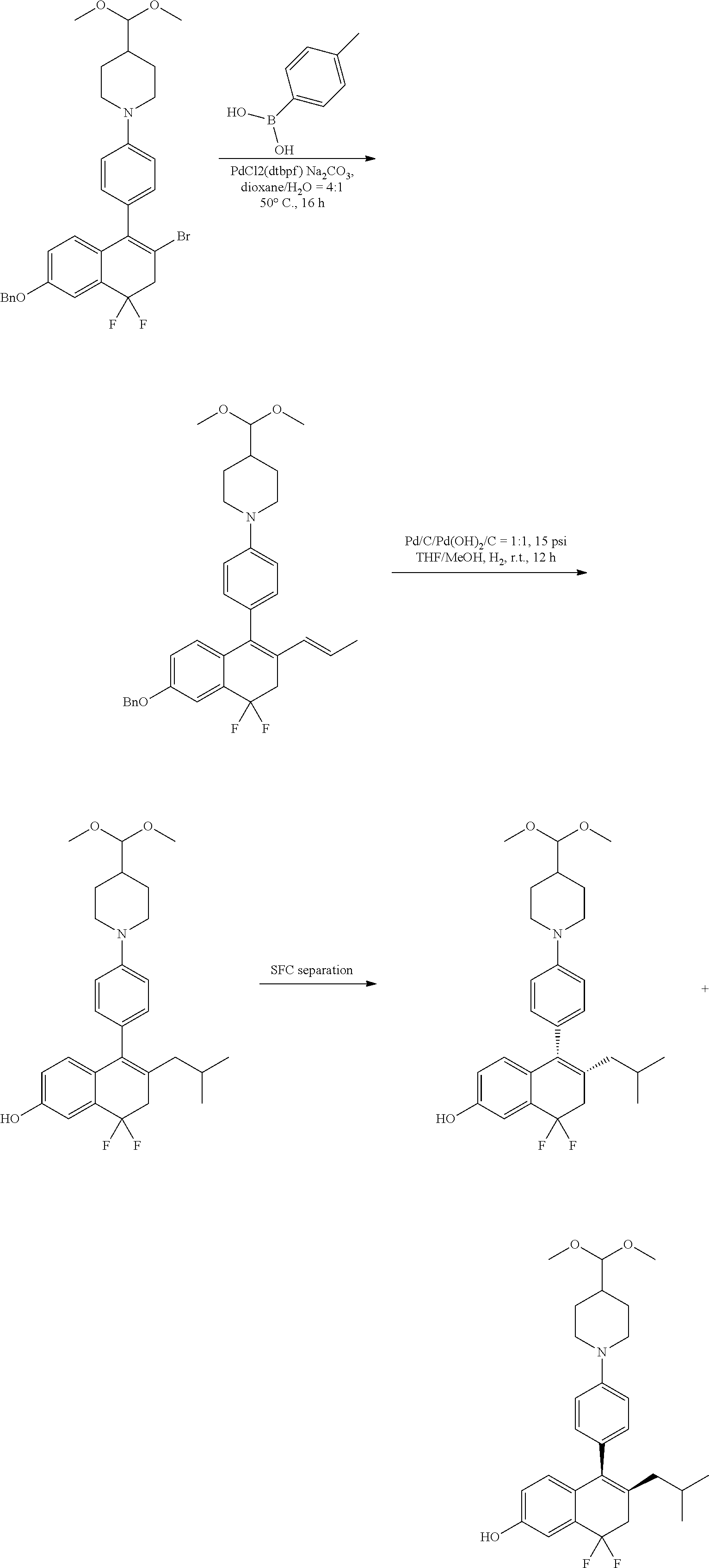

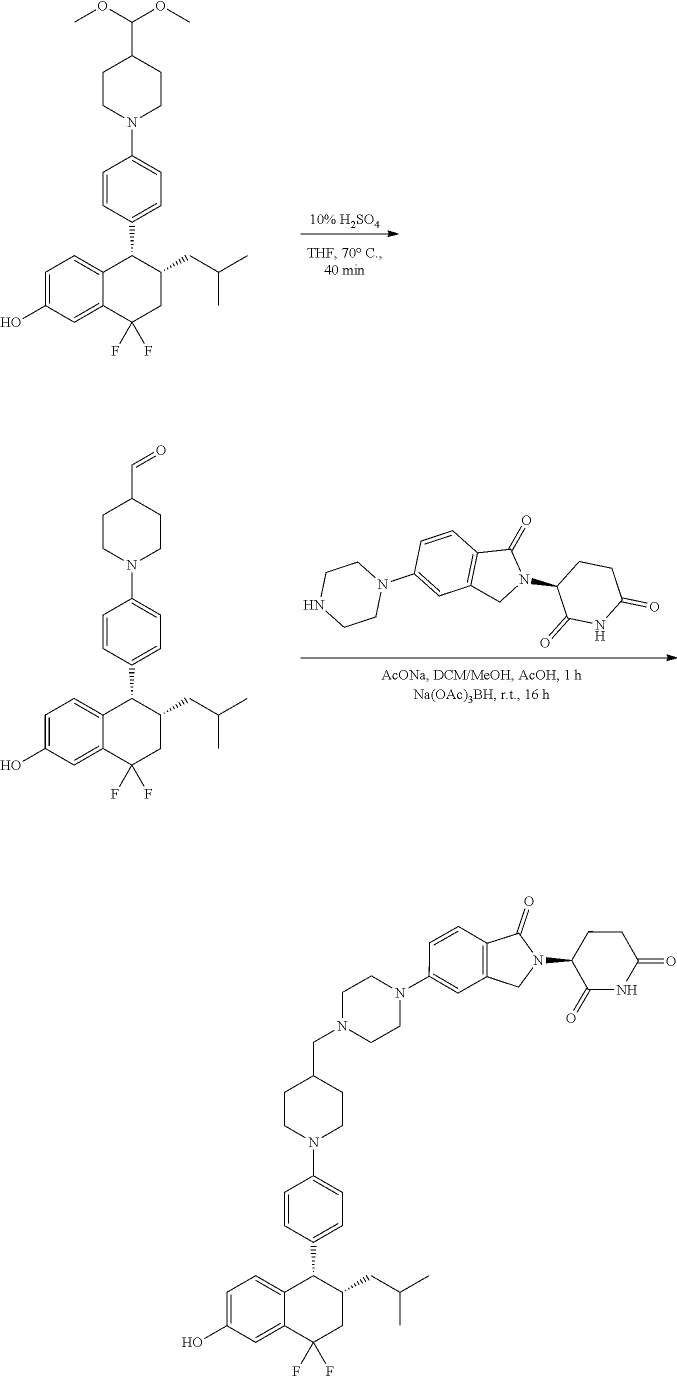 [SMILES: COC(OC)C1CCN(c2ccc([C@H]3c4ccc(O)cc4C(F)(F)C[C@H]3CC(C)C)cc2)CC1 ; CC(C)C[C@@H]1CC(F)(F)c2cc(O)ccc2[C@@H]1c1ccc(N2CCC(C=O)CC2)cc1 ; O=C1CC[C@H](N2Cc3cc(N4CCNCC4)ccc3C2=O)C(=O)N1 ; CC(C)C[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1]

Step 1. A mixture of 1,1′-Bis(di-t-butylphosphino)ferrocene palladium dichloride (78 mg, 0.1 Eq, 120 μmol), (2-methylprop-1-en-1-yl) boronic acid (120 mg, 1 Eq, 1 mmol), sodium carbonate (381 mg, 3 Eq, 3.6 mmol) 1-(4-(6-(benzyloxy)-2-bromo-4,4-difluoro-3,4-dihydronaphthalen-1-yl)phenyl)-4-(dimethoxymethyl)piperidine (700 mg, 1 Eq, 1.2 mmol) in 1,4-dioxane (8 mL) and H₂O (2 mL) was degassed and purged with N₂ for 3 times, and then the mixture was stirred at 50° C. for 16 hour under N₂ atmosphere. TLC (petroleum ether:ethyl acetate=5:1, R$=0.5 UV) showed one main new spot was observed. The reaction was quenched with water (100 mL) and extracted with ethyl acetate (150 mL*2). The organic layer was washed with brine (100 mL) and dried over Na₂SO₄ and concentrated in vacuum to give a white solid. The white solid was subjected to column chromatography over silica gel (gradient elution: 0-100% EtOAc). The desired fractions were collected, and concentrated to dryness in vacuum to give 1-(4-(6-(benzyloxy)-4,4-difluoro-2-(2-methylprop-1-en-1-yl)-3,4-dihydronaphthalen-1-yl)phenyl)-4-(dimethoxymethyl)piperidine (640 mg, 1.01 mmol, 84.7%, 88.7% purity) as a white solid.

LC-MS (ESI⁺) m/z: 560.29 (M+H)⁺.

Step 2. A mixture of 1-(4-(6-(benzyloxy)-4,4-difluoro-2-(2-methylprop-1-en-1-yl)-3,4-dihydronaphthalen-1-yl)phenyl)-4-(dimethoxymethyl)piperidine (640 mg, 1 Eq, 1 mmol), Pd/C (320 mg, 10% Wt, 0.3 Eq, 301 μmol), Pd(OH)₂/C (320 mg, 20% Wt, 0.4 Eq, 456 μmol) in MeOH (15 mL) and THF (15 mL) was degassed and purged with H₂ for 3 times, and then the mixture was stirred at 25° C. for 12 hour under H₂ atmosphere (15 psi). The reaction mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um); mobile phase: [CO2-i-PrOH (0.1% NH₃H₂O)]; B %: 30%-30%) to give (5S,6R)-5-(4-(4-(dimethoxymethyl)piperidin-1-yl)phenyl)-8,8-difluoro-6-isobutyl-5,6,7,8-tetrahydronaphthalen-2-ol (100 mg, 211 μmol, 18.5%) and (5R,6S)-5-(4-(4-(dimethoxymethyl)piperidin-1-yl)phenyl)-8,8-difluoro-6-isobutyl-5,6,7,8-tetrahydronaphthalen-2-ol (130 mg, 274 μmol, 24.0%) as yellow solid. LC-MS (ESI⁺) m/z: 474.2 (M+H)⁺.

Step 3. To a solution of (5S,6R)-5-(4-(4-(dimethoxymethyl)piperidin-1-yl)phenyl)-8,8-difluoro-6-isobutyl-5,6,7,8-tetrahydronaphthalen-2-ol (100 mg, 1 Eq, 211 μmol) in 10% H₂SO₄ (3 mL) and THF (3 mL) was stirred at 70° C. for 40 min. TLC (petroleum ether:ethyl acetate=3:1, R_{f}=0.4UV) showed a new spot was desired. The reaction was adjust to pH=˜8 with aq·NaHCO₃ (20 mL). Then the mixture was added water (30 mL) and extracted with ethyl acetate (30×2 mL). The combined organic layers were washed with brine (30 mL), dried over anhydrous Na₂SO₄, filtered and concentrated to dryness in vacuo to give 1-(4-((1S,2R)-4,4-difluoro-6-hydroxy-2-isobutyl-1,2,3,4-tetrahydronaphthalen-1-yl)phenyl)piperidine-4-carbaldehyde (90 mg, 0.21 mmol, 100%) as a yellow solid.

Step 4. To a solution of 1-(4-((1S,2R)-4,4-difluoro-6-hydroxy-2-isobutyl-1,2,3,4-tetrahydronaphthalen-1-yl)phenyl)piperidine-4-carbaldehyde (90 mg, 1 Eq, 211 μmol), (S)-3-(1-oxo-5-(piperazin-1-yl)isoindolin-2-yl)piperidine-2,6-dione, PhSO₃H salt (105 mg, 1 Eq, 211 μmol) in DCM (4 mL) and MeOH (4 mL) was added sodium acetate (52 mg, 3 Eq, 632 μmol) was stirred at 25° C. for 60 min. Then sodium triacetoxyborohydride (89 mg, 2 Eq, 421 μmol) and acetic acid (38 mg, 36 μL, 3 Eq, 632 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um; mobile phase: [water(FA)-ACN]; B % 20%-50%, 7 min) to give (S)-3-(5-(4-((1-(4-((1S,2R)-4,4-difluoro-6-hydroxy-2-isobutyl-1,2,3,4-tetrahydronaphthalen-1-yl)phenyl)piperidin-4-yl)methyl)piperazin-1-yl)-1-oxoisoindolin-2-yl)piperidine-2,6-dione (71.5 mg, 95.1 μmol, 45.2%, 98.365% purity) was obtained as a white solid. LC-MS (ESI⁺) m/z: 740.2 (M+H)⁺.

LCMS; calc. for C₄₃H₅₁F₂N₅O₄: 739.91, found: [M+H]⁺ 740.2.

HPLC: 98.365% purity at 220 nm.

¹H NMR (400 MHZ, DMSO-d₆) δ=10.96 (s, 1H), 9.66 (br s, 1H), 8.16 (s, 0.168H), 7.52 (d, J=8.6 Hz, 1H), 7.07 (s, 2H), 7.00 (s, 1H), 6.82 (br d, J=7.2 Hz, 4H), 6.70 (br d, J=8.6 Hz, 2H), 5.05 (dd, J=5.0, 13.2 Hz, 1H), 4.39-4.28 (m, 1H), 4.26-4.16 (m, 1H), 4.03 (br s, 1H), 3.62 (br $, 2H), 3.48-3.25 (m, 7H), 2.89 (br d, J=12.8 Hz, 1H), 2.65-2.52 (m, 4H), 2.43-2.33 (m, 1H), 2.21 (br d, J=6.9 Hz, 3H), 2.13-1.91 (m, 3H), 1.79 (br d, J=12.3 Hz, 4H), 1.19 (br d, J=9.4 Hz, 3H), 0.85 (dd, J=3.6, 6.4 Hz, 6H), 0.78-0.69 (m, 1H)

SFC: retention time, 2.180 min; Area, 96.310%.

### Example 20. Preparation of (1-197) (S)-3-(5-(4-((1-(4-((1R,2S)-4,4-difluoro-6-hydroxy-2-isobutyl-1,2,3,4-tetrahydronaphthalen-1-yl)phenyl)piperidin-4-yl)methyl)piperazin-1-yl)-1-oxoisoindolin-2-yl)piperidine-2,6-dione

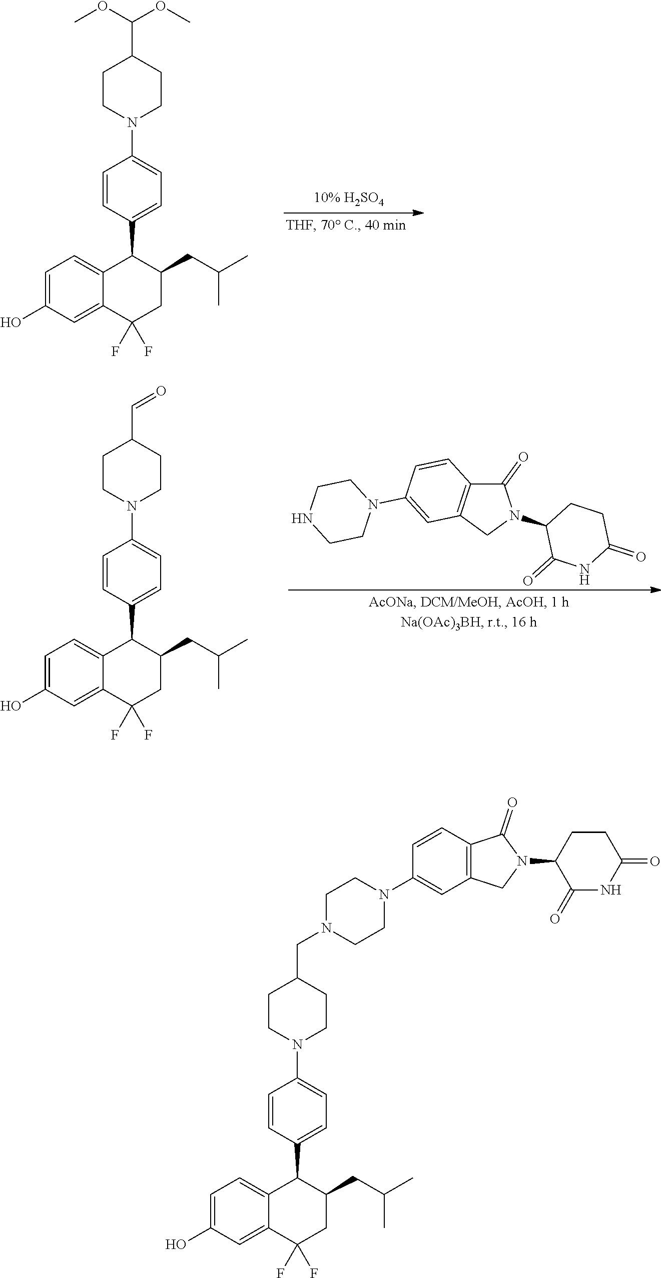

Step 1. To a solution of (5R,6S)-5-(4-(4-(dimethoxymethyl)piperidin-1-yl)phenyl)-8,8-difluoro-6-isobutyl-5,6,7,8-tetrahydronaphthalen-2-ol (130 mg, 1 Eq, 274 μmol) in 10% H₂SO₄ (3 mL) and THF (3 mL) was stirred at 70° C. for 40 min. TLC (petroleum ether:ethyl acetate=3:1, R_{f}=0.4UV) showed a new spot was formed. The reaction was adjust to pH=˜8 with aq·NaHCO₃ (15 mL). Then the mixture was added water (30 mL) and extracted with ethyl acetate (30×2 mL). The combined organic layers were washed with brine (30 mL), dried over anhydrous Na₂SO₄, filtered and concentrated to dryness in vacuum to give 1-(4-((1R,2S)-4,4-difluoro-6-hydroxy-2-isobutyl-1,2,3,4-tetrahydronaphthalen-1-yl)phenyl)piperidine-4-carbaldehyde (120 mg, 281 μmol) as a yellow solid.

Step 2. To a solution of 1-(4-((1R,2S)-4,4-difluoro-6-hydroxy-2-isobutyl-1,2,3,4-tetrahydronaphthalen-1-yl)phenyl)piperidine-4-carbaldehyde (120 mg, 1 Eq, 281 μmol), (S)-3-(1-oxo-5-(piperazin-1-yl)isoindolin-2-yl)piperidine-2,6-dione, PhSO₃H salt (141 mg, 1 Eq, 281 μmol) in DCM (4 mL) and MeOH (4 mL) was added sodium acetate (69 mg, 3 Eq. 842 μmol) was stirred at 25° C. for 60 mins, Then sodium triacetoxyborohydride (119 mg, 2 Eq, 561 μmol) and acetic acid (51 mg, 48 μL, 3 Eq, 842 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuum to give a yellow oil. The residue was purified by prep-HPLC (column: Welch Xtimate C18 150*30 mm*5 um; mobile phase: [water(FA)-ACN]; B % 20%-50%, 7 min) to give (S)-3-(5-(4-((1-(4-((1R,2S)-4,4-difluoro-6-hydroxy-2-isobutyl-1,2,3,4-tetrahydronaphthalen-1-yl)phenyl)piperidin-4-yl)methyl)piperazin-1-yl)-1-oxoisoindolin-2-yl)piperidine-2,6-dione (72.4 mg, 93.8 μmol, 33.4%, 95.865% purity) was obtained as a white solid. LC-MS (ESI⁺) m/z: 740.2 (M+H)⁺.

LCMS: calc. for C₄₃H₅₁F₂N₅O₄: 739.91, found: [M+H]⁺ 740.2.

HPLC: 95.865% purity at 220 nm.

¹H NMR (400 MHz, DMSO-d₆) δ=10.96 (s, 1H), 9.66 (br s, 1H), 8.16 (s, 0.165H), 7.52 (d, J=8.6 Hz, 1H), 7.07 (s, 2H), 7.00 (s, 1H), 6.82 (br d, J=6.9 Hz, 4H), 6.70 (d, J=8.6 Hz, 2H), 5.05 (dd, J=5.1, 13.2 Hz, 1H), 4.37-4.29 (m, 1H), 4.25-4.16 (m, 1H), 4.03 (br d, J=2.4 Hz, 1H), 3.62 (br d, J=1.0 Hz, 2H), 3.34 (br s, 8H), 2.90 (br s, 1H), 2.60 (br d, J=1.2 Hz, 3H), 2.37 (br dd, J=4.7, 13.3 Hz, 1H), 2.21 (br d, J=7.2 Hz, 3H), 2.14-1.92 (m, 3H), 1.80 (br d, J=12.8 Hz, 3H), 1.67 (br d, J=3.5 Hz, 1H), 1.19 (br d, J=9.4 Hz, 2H), 1.14-1.05 (m, 1H), 0.85 (dd, J=3.9, 6.4 Hz, 6H), 0.75 (br d, J=6.4 Hz, 1H)

SFC: retention time, 2.173 min; Area, 94.166%.

### Example 21. Preparation of (I-307) (S)-3-(5-(4-((1-(4-((1R,2R)-4,4-difluoro-6-hydroxy-2-phenyl-1,2,3,4-tetrahydronaphthalen-1-yl)-3-fluorophenyl)piperidin-4-yl)methyl)piperazin-1-yl)-1-oxoisoindolin-2-yl)piperidine-2,6-dione

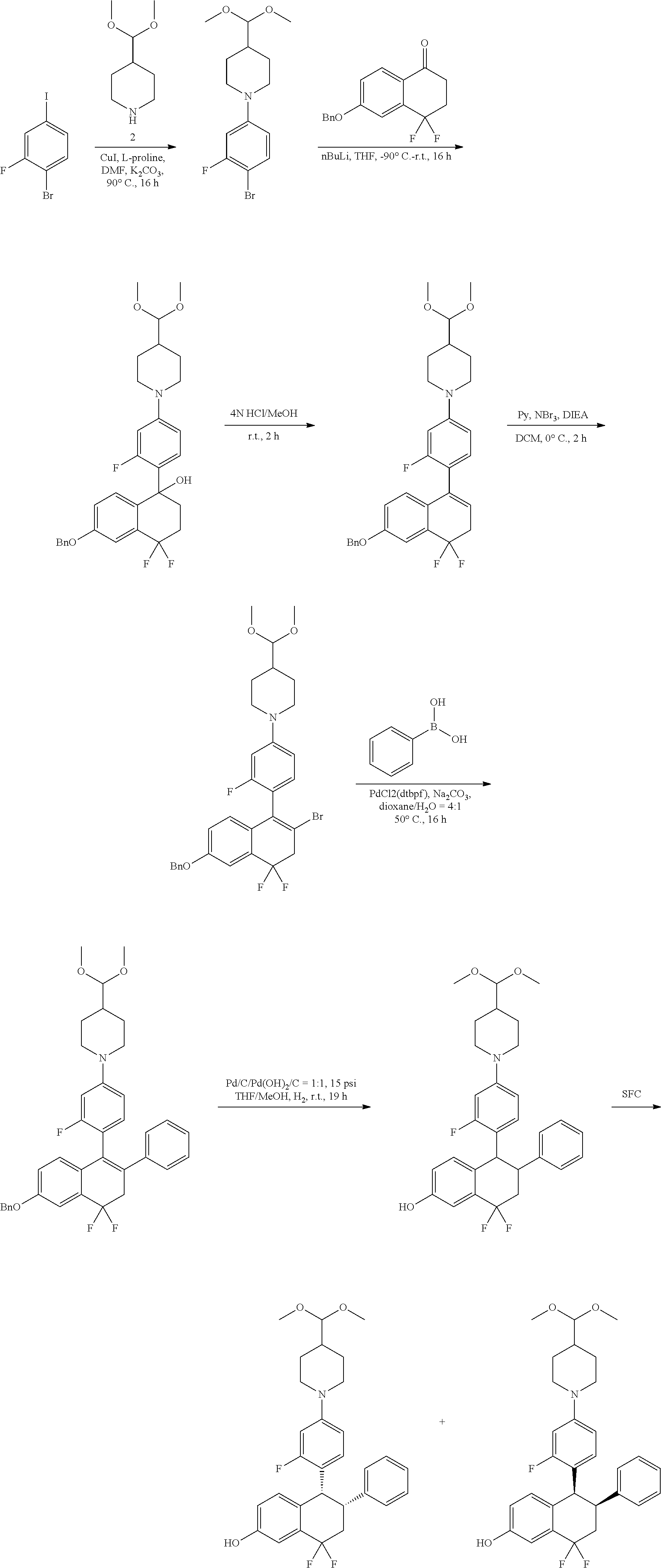

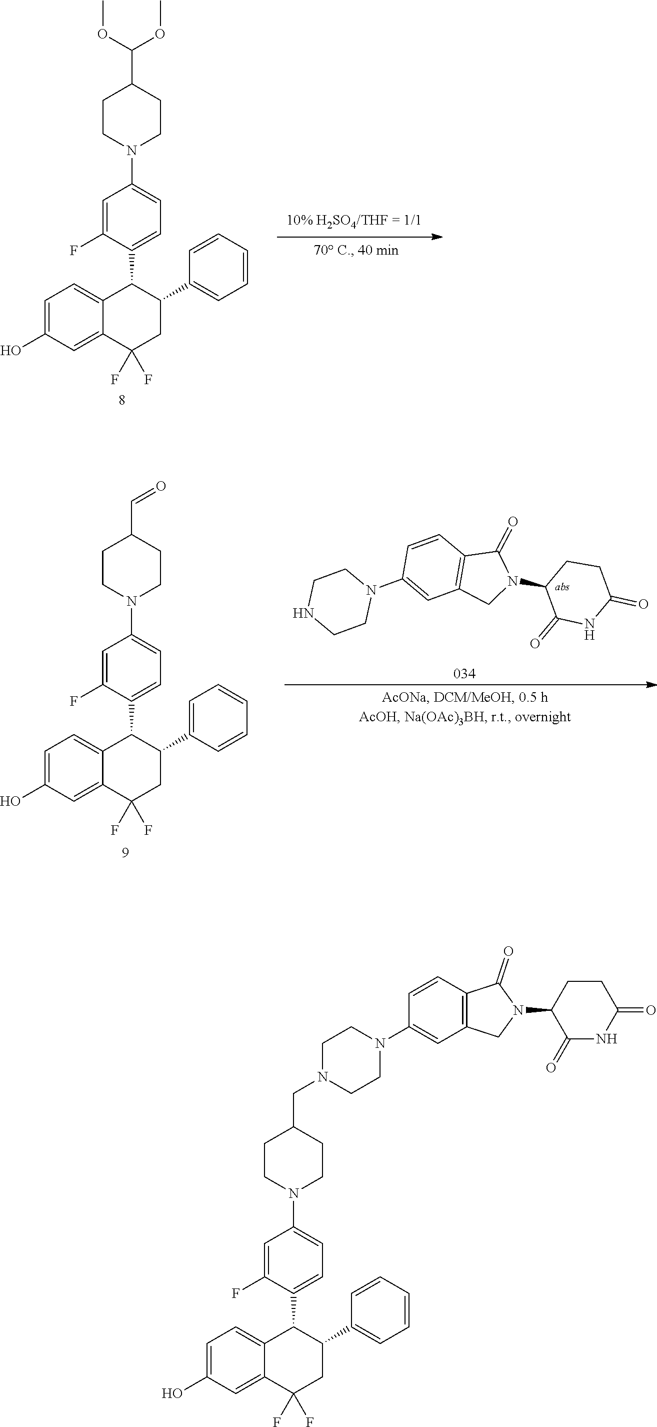 [SMILES: COC(OC)C1CCN(c2ccc([C@H]3c4ccc(O)cc4C(F)(F)C[C@H]3c3ccccc3)c(F)c2)CC1 ; O=CC1CCN(c2ccc([C@H]3c4ccc(O)cc4C(F)(F)C[C@H]3c3ccccc3)c(F)c2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccccc7)c(F)c6)CC5)CC4)ccc3C2=O)C(=O)N1]

Step 1. To a solution of 1-bromo-2-fluoro-4-iodobenzene (5 g, 16.6 mmol, 1 eq), 4-(dimethoxymethyl)piperidine (2.65 g, 16.62 mmol, 1 eq), iodocopper (633 mg, 3.32 mmol, 0.2 eq), potassium carbonate (4.59 g, 33.23 mmol, 2 eq), (2S)-pyrrolidine-2-carboxylic acid (765 mg, 6.7 mmol, 0.4 eq) was added in DMF (50 mL) and purged with N₂ for 3 times, the mixture was stirred at 90° C. for 16 hr under N₂. The resulting was treated with 100 mL H₂O, The reaction was extracted with ethyl acetate (200 mL*2). The organic layers were dried with anhydrous Na₂SO₄, filtered, and concentrated to give the crude product. The crude product was subjected to column chromatography over silica gel (gradient elution: 0-20% EtOAc). The desired fractions were collected, and concentrated to dryness in vacuo to give 1-(4-bromo-3-fluorophenyl)-4-(dimethoxymethyl)piperidine (2.5 g, 7.5 mmol, 45% yield) as a yellow oil. LC-MS (ESI⁺) m/z: 332.21 (M+H)⁺.

Step 2. To a mixture of nBuLi (2.5 M, 180.61 μL, 1.5 eq) was added into mixture of 1-(4-bromo-3-fluorophenyl)-4-(dimethoxymethyl)piperidine (2.4 g, 1 Eq. 7.2 mmol) in THF (30 mL) under N₂ at −68° C., and the mixture was stirred at −68° C. for 0.5 h under N₂ atmosphere, and then 6-(benzyloxy)-4,4-difluoro-3,4-dihydronaphthalen-1 (2H)-one (2.9 g, 1.4 Eq, 10 mmol) was added in the mixture at −68° C. and was stirred at 25° C. for 16 hr. TLC (petroleum ether:ethyl acetate=10:1, R_{f}=0.5, UV) showed one main new spot was observed. The reaction was quenched with sat. NH₄Cl aqueous (50 mL) and extracted with ethyl acetate (150 mL*2). The organic layer was washed with brine (50 mL) and dried over Na₂SO₄ and concentrated in vacuo to give a black solid. The black solid was subjected to column chromatography over silica gel (gradient elution: 0-10% EtOAc). The desired fractions were collected, and concentrated to dryness in vacuo to give 6-(benzyloxy)-1-(4-(4-(dimethoxymethyl)piperidin-1-yl)-2-fluorophenyl)-4,4-difluoro-1,2,3,4-tetrahydronaphthalen-1-ol (2.5 g, 4.6 mmol, 64%) as a yellow solid. LC-MS (ESI⁺) m/z: 541.61 (M+H)⁺.

Step 3. A mixture of 6-(benzyloxy)-1-(4-(4-(dimethoxymethyl)piperidin-1-yl)-2-fluorophenyl)-4,4-difluoro-1,2,3,4-tetrahydronaphthalen-1-ol (2 g, 1 Eq, 4 mmol) in 4N HCl/MeOH (20 mL) at r.t., and the reaction was stirred at 25° C. for 1 hour. TLC (petroleum ether:ethyl acetate=3:1, R_{f}=0.5 UV) showed a new spot was observed. The reaction was quenched with sat.aq. NaHCO₃ (100 mL) and extracted with ethyl acetate (150 mL*2). The organic layer was washed with brine (100 mL) and dried over Na₂SO₄ and concentrated in vacuum to give a white solid. The white solid was subjected to column chromatography over silica gel (gradient elution: 0-100% EtOAc). The desired fractions were collected, and concentrated to dryness in vacuum to give 1-(4-(6-(benzyloxy)-4,4-difluoro-3,4-dihydronaphthalen-1-yl)-3-fluorophenyl)-4-(dimethoxymethyl)piperidine (1.1 g, 2.1 mmol, 60%) as a white solid. LC-MS (ESI+) m/z: 523.60 (M+H)⁺.

Step 4. A mixture of 1-(4-(6-(benzyloxy)-4,4-difluoro-3,4-dihydronaphthalen-1-yl)-3-fluorophenyl)-4-(dimethoxymethyl)piperidine (1.1 g, 1 Eq, 2.1 mmol) and Diisopropylethylamine (0.4 g, 0.5 mL, 1.5 Eq, 3.2 mmol) in DCM (20 mL) at 0° C., and pyridinium bromide perbromide (0.67 g, 1 Eq, 2.1 mmol) was added to the mixture at 0° C., then the mixture was stirred at 0° C. for 2 hour. TLC (petroleum ether:ethyl acetate=3:1, R_{f}=0.5 UV) showed a new spot was observed. The reaction was treated with water (100 mL) and extracted with ethyl acetate (150 mL*2). The organic layer was washed with brine (100 mL) and dried over Na₂SO₄ and concentrated in vacuo to give a white solid. The white solid was subjected to column chromatography over silica gel (gradient elution: 0-100% EtOAc). The desired fractions were collected, and concentrated to dryness in vacuum to give 1-(4-(6-(benzyloxy)-2-bromo-4,4-difluoro-3,4-dihydronaphthalen-1-yl)-3-fluorophenyl)-4-(dimethoxymethyl)piperidine (860 mg, 1.4 mmol, 68%) as a white solid. LC-MS (EST′) m/z: 602.49 (M+H)⁺.

Step 5. A mixture of 1, l′-Bis(di-t-butylphosphino)ferrocene palladium dichloride (93 mg, 0.1 Eq, 143 μmol), phenylboronic acid (174 mg, 1 Eq, 1.43 mmol), sodium carbonate (454 mg, 3 Eq, 4 mmol) 1-(4-(6-(benzyloxy)-2-bromo-4,4-difluoro-3,4-dihydronaphthalen-1-yl)-3-fluorophenyl)-4-(dimethoxymethyl)piperidine (860 mg, 1 Eq, 1.43 mmol) in 1,4-dioxane (8 mL) and H₂O (2 mL) was degassed and purged with N₂ for 3 times, and then the mixture was stirred at 50° C. for 16 hour under N₂ atmosphere. TLC (petroleum ether:ethyl acetate=5:1, R/=0.5 UV) showed one main new spot was observed. The reaction was quenched with water (100 mL) and extracted with ethyl acetate (150 mL*2). The organic layer was washed with brine (100 mL) and dried over Na₂SO₄ and concentrated in vacuum to give a white solid. The white solid was subjected to column chromatography over silica gel (gradient elution: 0-100% EtOAc). The desired fractions were collected, and concentrated to dryness in vacuum to give 1-(4-(6-(benzyloxy)-4,4-difluoro-2-phenyl-3,4-dihydronaphthalen-1-yl)-3-fluorophenyl)-4-(dimethoxymethyl)piperidine (810 mg, 1.35 mmol, 94.6%) as a white solid. LC-MS (ESI⁺) m/z: 413.3 (M+H)⁺.

Step 6. A mixture of 1-(4-(6-(benzyloxy)-4,4-difluoro-2-phenyl-3,4-dihydronaphthalen-1-yl)-3-fluorophenyl)-4-(dimethoxymethyl)piperidine (640 mg, 1 Eq, 1 mmol), Pd/C (320 mg, 10% Wt, 0.3 Eq, 301 μmol), Pd(OH)₂/C (320 mg, 20% Wt, 0.427 Eq, 456 μmol) in MeOH (15 mL) and THF (15 mL) was degassed and purged with H₂ for 3 times, and then the mixture was stirred at 50° C. for 12 hour under H₂ atmosphere (15 psi). The reaction mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um); mobile phase: [CO₂-EtOH (0.1% NH₃H₂O)]; B %: 30%-30%) to give (5R,6R)-5-(4-(4-(dimethoxymethyl)piperidin-1-yl)-2-fluorophenyl)-8,8-difluoro-6-phenyl-5,6,7,8-tetrahydronaphthalen-2-ol (80 mg, 0.16 mmol, 15%) and (5S,6S)-5-(4-(4-(dimethoxymethyl)piperidin-1-yl)-2-fluorophenyl)-8,8-difluoro-6-phenyl-5,6,7,8-tetrahydronaphthalen-2-ol (80 mg, 0.16 mmol, 15%) as yellow solid. LC-MS (ESI+) m/z: 512.3 (M+H)⁺.

Step 7. To a solution of (5R,6R)-5-(4-(4-(dimethoxymethyl)piperidin-1-yl)-2-fluorophenyl)-8,8-difluoro-6-phenyl-5,6,7,8-tetrahydronaphthalen-2-ol (80 mg, 1 Eq, 0.16 mmol) in 10% H₂SO₄ (3 mL) and THF (3 mL) was stirred at 70° C. for 40 min. TLC (petroleum ether:ethyl acetate=3:1, R_{f}=0.4UV) showed a new spot was desired. The reaction was adjust to pH=˜8 with aq. NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1R,2R)-4,4-difluoro-6-hydroxy-2-phenyl-1,2,3,4-tetrahydronaphthalen-1-yl)-3-fluorophenyl)piperidine-4-carbaldehyde (50 mg, 0.11 mmol, 69%) as a yellow solid.

Step &. To a solution of 1-(4-((1R,2R)-4,4-difluoro-6-hydroxy-2-phenyl-1,2,3,4-tetrahydronaphthalen-1-yl)-3-fluorophenyl)piperidine-4-carbaldehyde (50 mg, 1 Eq, 107 μmol), (S)-3-(1-oxo-5-(piperazin-1-yl)isoindolin-2-yl)piperidine-2,6-dione, PhSO₃H salt (54 mg, 1 Eq, 107 μmol) in DCM (4 mL) and MeOH (4 mL) was added sodium acetate (26 mg, 18 μL, 3 Eq, 322 μmol) was stirred at 25° C. for 30 mins, Then Na(OAc)₃BH (46 mg, 2 Eq, 215 μmol) and acetic acid (19.3 mg, 19 μL, 3 Eq, 322 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuum to give a yellow oil. The residue was purified by prep-HPLC (column: Welch Xtimate C18 150*30 mm*5 um; mobile phase: [water(FA)-ACN]; B % 25%-55%, 7 min) to give (S)-3-(5-(4-((1-(4-((1R,2R)-4,4-difluoro-6-hydroxy-2-phenyl-1,2,3,4-tetrahydronaphthalen-1-yl)-3-fluorophenyl)piperidin-4-yl)methyl)piperazin-1-yl)-1-oxoisoindolin-2-yl)piperidine-2,6-dione (40.9 mg, 52.2 μmol, 48.6%, 99.282% Purity) was obtained as a white solid.

LC-MS (ESI⁺) m/z: 778.2 (M+H)⁺.

LCMS: calc. for C₄₅H₄₆F₃N₅O₄: 777.89, found: [M+H]⁺ 778.2.

HPLC: 99.282% purity at 220 nm,

¹H NMR (400 MHZ, DMSO-d₆) δ=10.96 (s, 1H), 9.83 (br d, J=3.5 Hz, 1H), 8.15 (s, 0.582H), 7.52 (d, J=8.7 Hz, 1H), 7.22-7.13 (m, 3H), 7.11-7.02 (m, 3H), 6.96-6.83 (m, 4H), 6.55 (dd, J=1.9, 8.7 Hz, 1H), 6.38-6.27 (m, 2H), 5.05 (dd, J=5.1, 13.3 Hz, 1H), 4.57 (br d, J=2.9 Hz, 1H), 4.39-4.28 (m, 1H), 4.26-4.15 (m, 1H), 3.60 (br d, J=12.2 Hz, 4H), 3.28 (br s, 7H), 2.97-2.85 (m, 1H), 2.73 (brs, 1H), 2.57 (br s, 3H), 2.34 (br s, 2H), 2.19 (br d, J=6.7 Hz, 2H), 1.97 (br d, J=5.2 Hz, 1H), 1.74 (br d, J=13.0 Hz, 3H), 1.13 (br d, J=11.3 Hz, 2H)

SFC: retention time, 2.485 min; Area, 96.830%.

### Example 22. Preparation of (1-199) (S)-3-(5-(4-((1-(4-((1S,2S)-4,4-difluoro-6-hydroxy-2-phenyl-1,2,3,4-tetrahydronaphthalen-1-yl)-3-fluorophenyl)piperidin-4-yl)methyl)piperazin-1-yl)-1-oxoisoindolin-2-yl)piperidine-2,6-dione

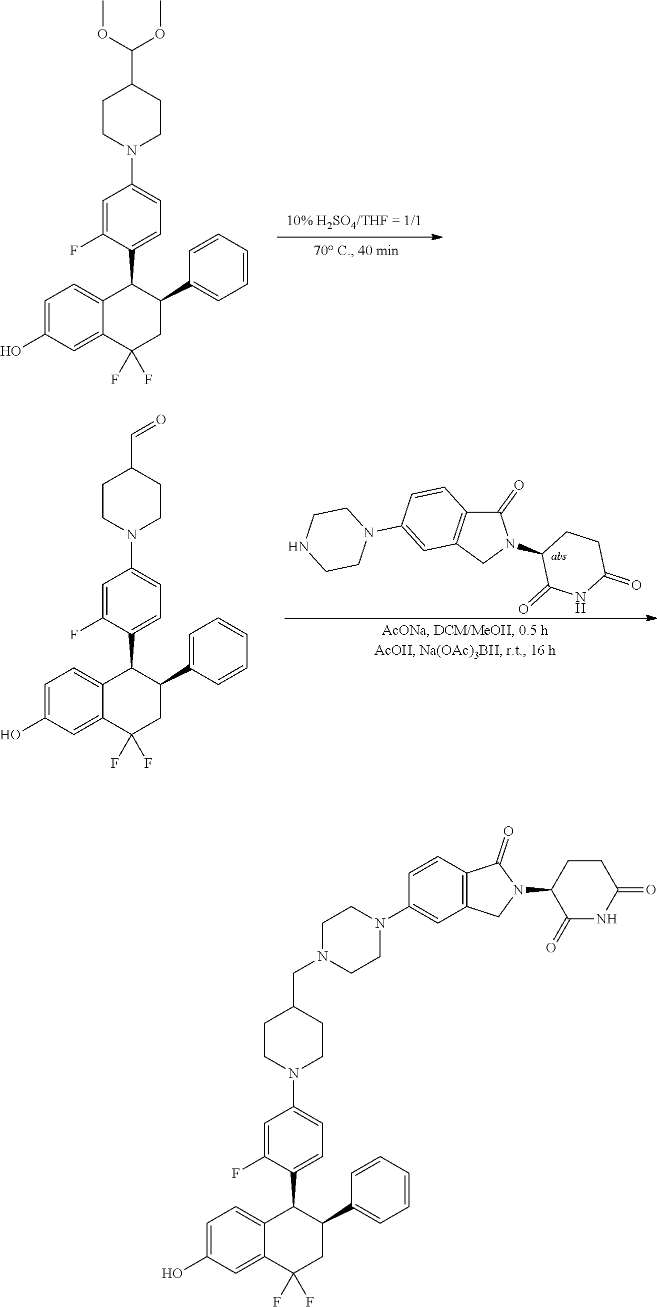

Step 1. To a solution of (5S,6S)-5-(4-(4-(dimethoxymethyl)piperidin-1-yl)-2-fluorophenyl)-8,8-difluoro-6-phenyl-5,6,7,8-tetrahydronaphthalen-2-ol (80 mg, 1 Eq, 0.16 mmol) in 10% H₂SO₄ (3 mL) and THF (3 mL) was stirred at 70° C. for 40 min. TLC (petroleum ether:ethyl acetate=3:1, R_{f}=0.4, UV) showed a new spot was desired. The reaction was adjust to pH=˜8 with sat.aq·NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuum to give 1-(4-((1S,2S)-4,4-difluoro-6-hydroxy-2-phenyl-1,2,3,4-tetrahydronaphthalen-1-yl)-3-fluorophenyl)piperidine-4-carbaldehyde (100 mg, 215 μmol, crude) as a yellow solid.

Step 2. To a solution of 1-(4-((1S,2S)-4,4-difluoro-6-hydroxy-2-phenyl-1,2,3,4-tetrahydronaphthalen-1-yl)-3-fluorophenyl)piperidine-4-carbaldehyde (100 mg, 1 Eq, 215 μmol), (S)-3-(1-oxo-5-(piperazin-1-yl)isoindolin-2-yl)piperidine-2,6-dione, PhSO₃H salt (108 mg, 1 Eq, 215 μmol) in DCM (4 mL) and MeOH (4 mL) was added sodium acetate (53 mg, 3 Eq, 644 μmol) was stirred at 25° C. for 30 mins. Then NaBH(OAc); (91 mg, 2 Eq, 430 μmol) and acetic acid (39 mg, 37 μL, 3 Eq, 644 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuum to give a yellow oil. The residue was purified by prep-HPLC (column: Welch Xtimate C18 150*30 mm*5 um; mobile phase: [water (NH₃H₂O+NH₄HCO₃)-ACN]; B % 70%-100%, 7 min) to give (S)-3-(5-(4-((1-(4-((1S,2S)-4,4-difluoro-6-hydroxy-2-phenyl-1,2,3,4-tetrahydronaphthalen-1-yl)-3-fluorophenyl)piperidin-4-yl)methyl)piperazin-1-yl)-1-oxoisoindolin-2-yl)piperidine-2,6-dione (28.1 mg, 35.4 μmol, 16.5%, 97.864% purity) was obtained as a white solid. LC-MS (ESI⁺) m/z: 778.2 (M+H)⁺.

LCMS: calc. for C₄₅H₄₆F₃N₅O₄: 777.89, found: [M+H]⁺ 778.6.

HPLC: 97.864% purity at 220 nm.

¹H NMR (400 MHz, DMSO-d₆) δ=10.96 (s, 1H), 9.77 (s, 1H), 7.52 (d, J=8.6 Hz, 1H), 7.16 (br s, 3H), 7.07 (s, 3H), 6.86 (s, 4H), 6.55 (dd, J=2.0, 8.7 Hz, 1H), 6.37-6.27 (m, 2H), 5.05 (dd, J=4.9, 13.3 Hz, 1H), 4.57 (br d, J=3.0 Hz, 1H), 4.39-4.28 (m, 1H), 4.26-4.15 (m, 1H), 3.69 (br d, J=13.4 Hz, 1H), 3.60 (br d, J=11.9 Hz, 2H), 3.40-3.27 (m, 8H), 2.90 (s, 1H), 2.74 (s, 1H), 2.57 (br s, 3H), 2.35 (br d, J=4.6 Hz, 2H), 2.18 (br d, J=6.8 Hz, 2H), 2.02-1.89 (m, 1H), 1.73 (br s, 3H), 1.12 (br d, J=11.2 Hz, 2H)

SFC: retention time, 2.581 min; Area, 95.294%.

### Example 23. Preparation of (I-405) (S)-3-(5-(4-((1-(5-((R)-4,4-difluoro-6-hydroxy-1,2,3,4-tetrahydronaphthalen-1-yl)-4-methoxy-[1,1′-biphenyl]-2-yl)piperidin-4-yl)methyl)piperazin-1-yl)-1-oxoisoindolin-2-yl)piperidine-2,6-dione

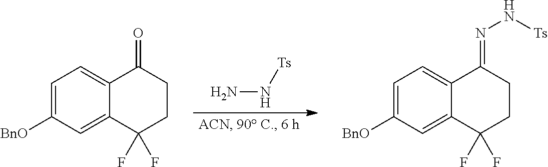 [SMILES: O=C1CCC(F)(F)c2cc(OCc3ccccc3)ccc21 ; Cc1ccc(S(=O)(=O)NN)cc1 ; Cc1ccc(S(=O)(=O)N/N=C2\CCC(F)(F)c3cc(OCc4ccccc4)ccc32)cc1]

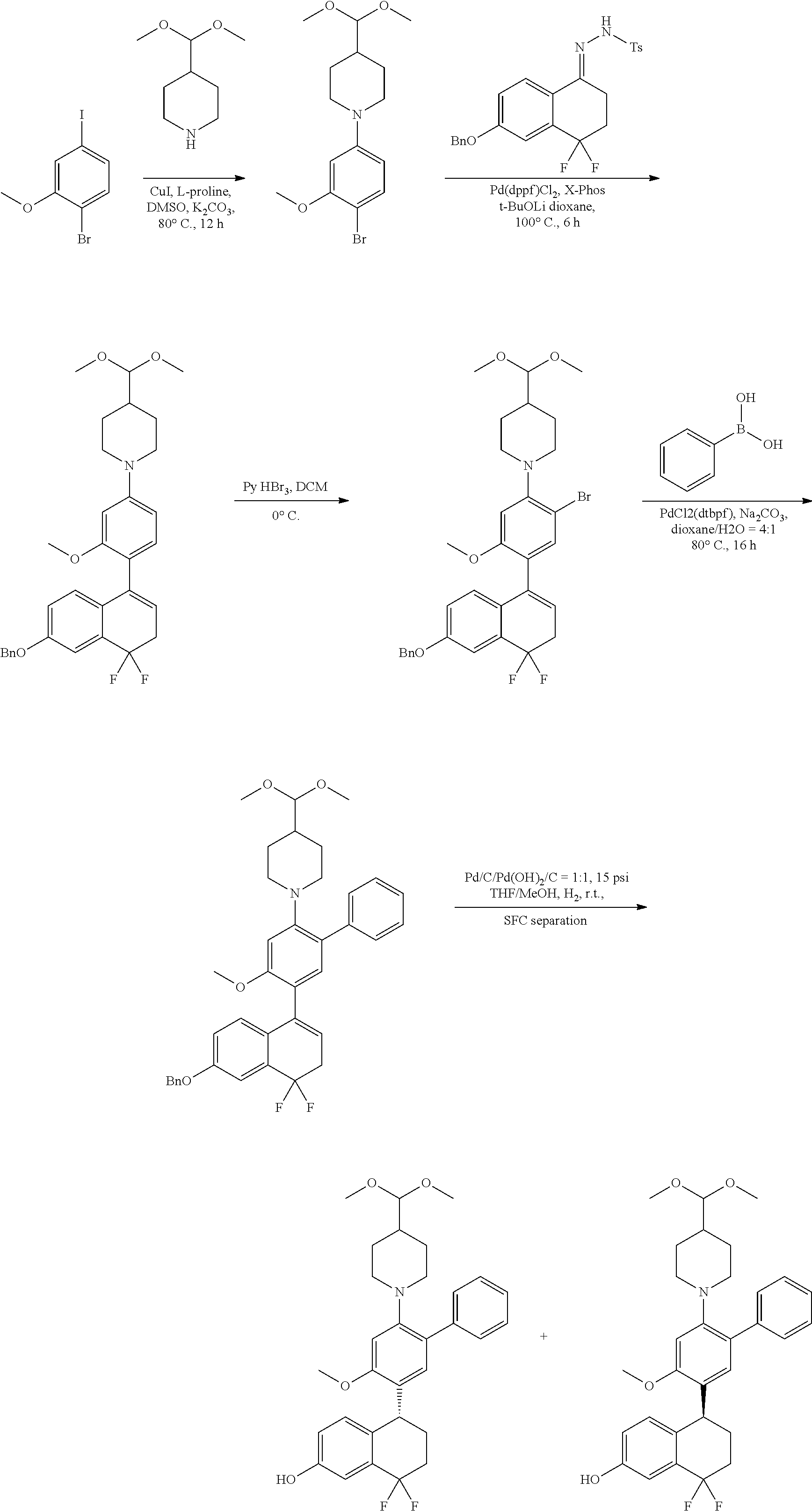

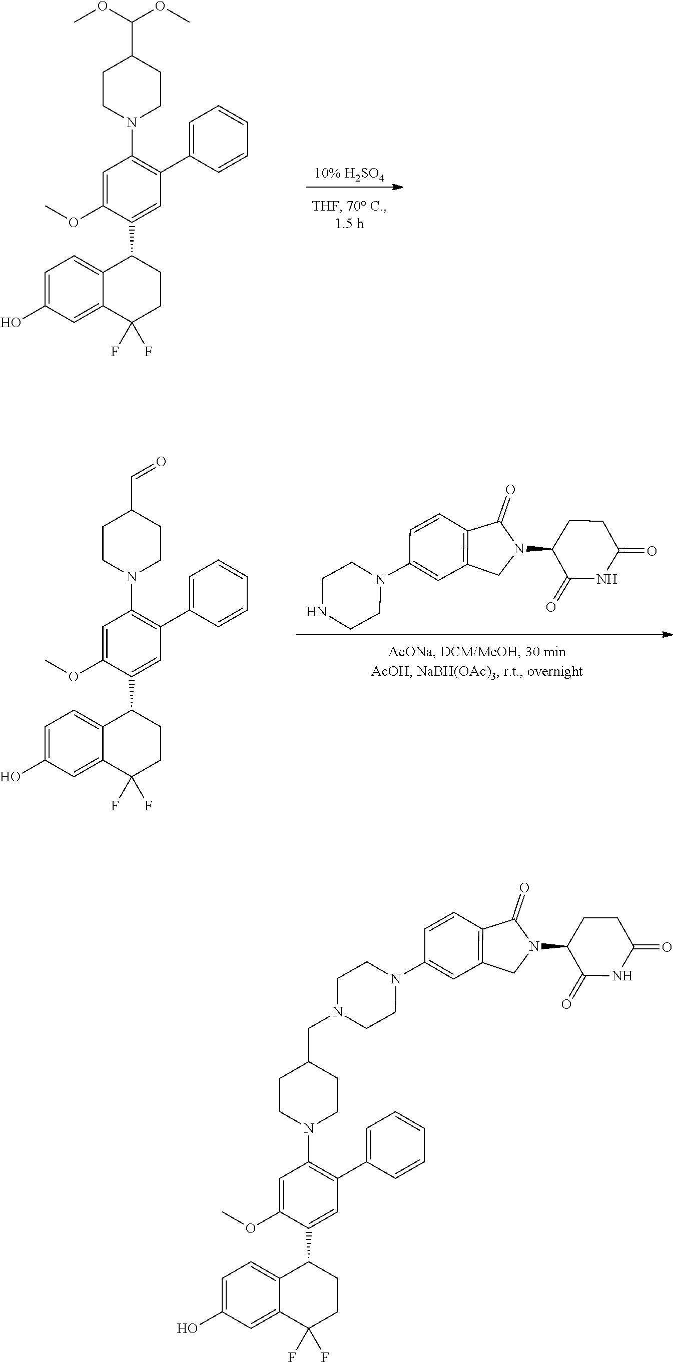 [SMILES: COc1cc(N2CCC(C(OC)OC)CC2)c(-c2ccccc2)cc1[C@@H]1CCC(F)(F)c2cc(O)ccc21 ; COc1cc(N2CCC(C=O)CC2)c(-c2ccccc2)cc1[C@@H]1CCC(F)(F)c2cc(O)ccc21 ; O=C1CC[C@H](N2Cc3cc(N4CCNCC4)ccc3C2=O)C(=O)N1 ; COc1cc(N2CCC(CN3CCN(c4ccc5c(c4)CN([C@H]4CCC(=O)NC4=O)C5=O)CC3)CC2)c(-c2ccccc2)cc1[C@@H]1CCC(F)(F)c2cc(O)ccc21]

Step 1. To a mixture of 6-(benzyloxy)-4,4-difluoro-3,4-dihydronaphthalen-1 (2H)-one (5.00 g, 1 Eq, 17.3 mmol) and 4-methylbenzenesulfonic acid hydrazide (3.88 g, 1.2 Eq, 20.8 mmol) in acetonitrile (60 mL), then the mixture was stirred at 90° C. for 6 hour. LCMS showed 80% desired MS. TLC (petroleum ether:ethyl acetate=5:1, R_{f}=0.5 UV) showed one main new spot was observed. The reaction was quenched with water (100 mL) and extracted with ethyl acetate (200 mL*2). The organic layer was washed with brine (50 mL) and dried over Na₂SO₄ and concentrated in vacuum to give a white solid. The white solid was subjected to column chromatography over silica gel (gradient elution: 0-100% EtOAc). The desired fractions were collected, and concentrated to dryness in vacuum to give (E)-N′-(6-(benzyloxy)-4, 4-difluoro-3,4-dihydronaphthalen-1 (2H)-ylidene)-4-methylbenzenesulfonohydrazide (7.5 g, 16 mmol, 95%) as a white solid. LC-MS (ESI+) m/z: 457.2 (M+H)⁺.

Step 2. To a solution of 1-bromo-4-iodo-2-methoxybenzene (5.00 g, 1 Eq, 16.0 mmol) and 4-(dimethoxymethyl) piperdine (2.54 g, 2.52 mL, 1 Eq, 16.0 mmol) in DMSO (50 mL) was added cuprous iodide (609 mg, 0.2 Eq, 3.20 mmol), (S)-(−)-proline (736 mg, 0.4 Eq, 6.39 mmol) and potassium carbonate (4.42 g, 2 Eq, 32.0 mmol). The mixture was stirred at 80° C. for 12 hour. LCMS showed 52.7% desired MS was detected. TLC (petroleum ether:ethyl acetate=10:1, UV, R_{f}=0.3) showed one main new spot was observed. The reaction was quenched with water (300 mL) and extracted with ethyl acetate (300 mL*2). The organic layer was washed with brine (50 mL) and dried over Na₂SO₄ and concentrated in vacuo to give 1-(4-bromo-3-methoxyphenyl)-4-(dimethoxymethyl) piperdine as a brown oil. The brown oil was subjected to column chromatography over silica gel (gradient elution: 0-15% EtOAc). The desired fractions were collected, and concentrated to dryness in vacuum to give 1-(4-bromo-3-methoxyphenyl)-4-(dimethoxymethyl) piper dine (3.6 g, 9.4 mmol, 59% yield, 90% purity) as a white solid. LC-MS (ESI+) m/z: 346.1 (M+H)⁺.

Step 3. To a solution of lithium 2-methyl-2-propanolate (631 mg, 733 μL, 2 Eq. 7.89 mmol) and 2-(dicyclohexylphosphanyl)-2′,4′,6′-tris(isopropyl) biphenyl (940 mg, 0.5 Eq, 1.97 mmol) in dioxane (80 mL) was added 1-(4-bromo-3-methoxyphenyl)-4-(dimethoxymethyl)piperidine (1.36 g, 1 Eq, 3.94 mmol), (E)-N′-(6-(benzyloxy)-4,4-difluoro-3,4-dihydronaphthalen-1 (2H)-ylidene)-4-methylbenzenesulfonohydrazide (1.80 g, 1 Eq, 3.94 mmol) and Pd(dppf)Cl₂-DCM adduct (322 mg, 0.1 Eq, 394 μmol). The mixture was stirred at 100° C. for 6 hour. TLC (petroleum ether:ethyl acetate=2:1, UV) showed one main new spot was observed. 100 mL water was added, and the mixture was extracted with dichloromethane (150 mL×2). The combined organic layers were washed with brine (100 mL), dried over anhydrous Na₂SO₄, filtered and concentrated to dryness in vacuo to give a yellow oil. The yellow oil was purified by flash column (petroleum ether/ethyl acetate from 100/0 to 0/100) and the organic layer was concentrated in vacuo to give 1-(4-(6-(benzyloxy)-4,4-difluoro-3,4-dihydronaphthalen-1-yl)-3-methoxyphenyl)-4-(dimethoxymethyl)piperidine-1-(4-(6-(benzyloxy)-4-fluoro-3,4-dihydronaphthalen-1-yl)-3-methoxyphenyl)-4-(dimethoxymethyl)piperidine (1.70 g, 1.61 mmol, 40.9%) as a yellow solid. LC-MS (ESI+) m/z: 536.0 (M+H)⁺.

Step 4. A solution of pyridinium bromide perbromide (0.36 g, 0.7 Eq, 1.1 mmol) was added to 1-(4-(6-(benzyloxy)-4,4-difluoro-3,4-dihydronaphthalen-1-yl)-3-methoxyphenyl)-4-(dimethoxymethyl)piperidine (1.7 g, 1 Eq, 1.6 mmol) in DCM (20 mL) was stirred at 0° C. for 1 hour. LCMS showed 19% desired MS was detected. 50 mL water was added, and the mixture was extracted with dichloromethane (100 mL×2). The combined organic layers were washed with brine (10 mL), dried over anhydrous Na₂SO₄, filtered and concentrated to dryness in vacuo to give a yellow oil. The yellow solid was purified by preparative high-performance liquid chromatography. Condition: Column: Xtimate C18 150*40 mm*5 um A: water(FA); B: ACN; Gradient Time (min) 9; 100% B hold Time (min) 2, Flow Rate (ml/min) 30. The pure fractions were collected and the solvent was evaporated under vacuum. The aqueous layer was lyophilized to dryness to give 1-(4-(6-(benzyloxy)-4, 4-difluoro-3,4-dihydronaphthalen-1-yl)-2-bromo-5-methoxyphenyl)-4-(dimethoxymethyl)piperidine (500 mg, 813.6 μmol, 50%) as a yellow solid.

Step 5. To a mixture of 1, l′-Bis(di-t-butylphosphino)ferrocene palladium dichloride (53 mg, 0.1 Eq, 81.4 μmol), Na₂CO₃ (259 mg, 3 Eq, 2.44 mmol) and 1-(4-(6-(benzyloxy)-4,4-difluoro-3,4-dihydronaphthalen-1-yl)-2-bromo-5-methoxyphenyl)-4-(dimethoxymethyl)piperidine (500 mg, 1 Eq, 813.6 μmol), phenylboronic acid (148.81 mg, 1.5 Eq, 1.2204 mmol) in Dioxane (10 mL) and water (2 mL) was stirred at 80° C. for 12 h. LCMS showed 77% desired MS was detected. TLC (petroleum ether:ethyl acetate=5:1, UV) showed one main new spot was observed. 50 mL water was added, and the mixture was extracted with dichloromethane (100 mL×2). The combined organic layers were washed with brine (100 mL), dried over anhydrous Na₂SO₄, filtered and concentrated to dryness in vacuo to give a yellow oil. The yellow oil was purified by flash column (petroleum ether/ethyl acetate from 100/0 to 0/100) and the organic layer was concentrated in vacuo to give 1-(5-(6-(benzyloxy)-4,4-difluoro-3,4-dihydronaphthalen-1-yl)-4-methoxy-[1,1′-biphenyl]-2-yl)-4-(dimethoxymethyl)piperidine (270.0 mg, 0.34 mmol, 42% yield, 78% purity) as a yellow solid. LC-MS (ESI+) m/z: 612.2 (M+H)⁺.

Step 6. To a solution of 1-(5-(6-(benzyloxy)-4,4-difluoro-3,4-dihydronaphthalen-1-yl)-4-methoxy-[1,1′-biphenyl]-2-yl)-4-(dimethoxymethyl)piperidine (260 mg, 1 Eq, 425.02 μmol) in MeOH (5 mL) and THF (5 mL) was added Pd/C (250 mg, 10% Wt.) under N₂ atmosphere. The suspension was degassed and purged with dihydrogen for three times. The mixture was stirred under 15 psi dihydrogen (859 μg, 1 Eq, 425.02 μmol) at 50° C. for 16 hr. LCMS showed 74.8% desired MS was detected. The mixture was filtered and concentrated under reduced pressure to give the residue as a yellow solid. The residue was concentrated under reduced pressure to give desired compound as a yellow oil, which was further separated by SFC (condition: column: DAICEL CHIRALPAK AD (250 mm*30 mm, 10 um)); mobile phase: CO₂-EtOH (0.1% NH₃H₂O); B %: 35%-35%) to give (R)-5-(6-(4-(dimethoxymethyl)piperidin-1-yl)-4-methoxy-[1,1′-biphenyl]-3-yl)-8,8-difluoro-5,6,7,8-tetrahydronaphthalen-2-ol (80.0 mg, 153 μmol, 35.9%) and (S)-5-(6-(4-(dimethoxymethyl)piperidin-1-yl)-4-methoxy-[1,1′-biphenyl]-3-yl)-8,8-difluoro-5,6,7,8-tetrahydronaphthalen-2-ol (72.0 mg, 138 μmol, 32.4%) as a white solid LC-MS (ESI+) m/z: 524.3 (M+H)⁺.

Step 7. To a solution of (R)-5-(6-(4-(dimethoxymethyl)piperidin-1-yl)-4-methoxy-[1, l′-biphenyl]-3-yl)-8,8-difluoro-5,6,7,8-tetrahydronaphthalen-2-ol (80.00 mg, 1 Eq, 152.8 μmol) in THF (4 mL) and 10% H₂SO₄ (4 mL). The mixture was stirred at 70° C. for 1.5 hour. LCMS showed 80.5% desired MS was detected. The reaction mixture was quenched by addition H₂O 50 mL at 25° C., and then diluted with 50 mL H₂O and extracted with ethyl acetate (50 mL*2). The combined organic layers were washed with 50 mL brine and dried over Na₂SO₄, filtered and concentrated under reduced pressure to give (S)-1-(5-(4,4-difluoro-6-hydroxy-1,2,3,4-tetrahydronaphthalen-1-yl)-4-methoxy-[1,1′-biphenyl]-2-yl)piperidine-4-carbaldehyde (75 mg, 0.16 mmol, 100%) as a residue. LC-MS (ESI+) m/z: 456.1 (M+H)⁺.

Step 8. To a solution of (R)-1-(5-(4,4-difluoro-6-hydroxy-1,2,3,4-tetrahydronaphthalen-1-yl)-4-methoxy-[1,1′-biphenyl]-2-yl)piperidine-4-carbaldehyde (70.0 mg, 1 Eq, 147 μmol), (S)-3-(1-oxo-5-(piperazin-1-yl)isoindolin-2-yl)piperidine-2,6-dione benzenesulfonate (85.6 mg, 1.2 Eq, 176 μmol) in DCM (2 mL), MeOH (2 mL) was added sodium acetate (36.1 mg, 3 Eq, 440 μmol) was stirred at 25° C. for 30 mins, Then sodium triacetoxyborohydride (62.1 mg, 2 Eq, 293 μmol) and acetic acid (26.4 mg, 3 Eq, 440 μmol) was added. The mixture was stirred at 25° C. for 16 hour. LCMS showed 72.9% desired MS was detected. The reaction mixture was concentrated under reduced pressure to remove solvent. The residue was purified by prep-HPLC (column: Welch Xtimate C18 150*30 mm*5 um phase: [water(FA)-ACN]; B %: 17%-57%, 9 min to give (S)-3-(5-(4-((1-(5-((R)-4,4-difluoro-6-hydroxy-1,2,3,4-tetrahydronaphthalen-1-yl)-4-methoxy-[1,1′-biphenyl]-2-yl)piperidin-4-yl)methyl)piperazin-1-yl)-1-oxoisoindolin-2-yl)piperidine-2,6-dione (29.3 mg, 35.2 μmol, 24.0%, 94.8% purity) was obtained as a white solid.

LCMS: calc. for C₄₆H₄₉F₂N₅O₅: 789.92, found: [M+H]⁺ 790.4.

HPLC: 94.833% purity at 220 nm.

¹H NMR (400 MHz, DMSO-d₆) δ=10.96 (s, 1H) 9.13-10.22 (m, 1H) 8.18 (s, 0.510H) 7.52 (d, J=8.58 Hz, 1H) 7.40-7.44 (m, 2H) 7.33 (t, J=7.63 Hz, 2H) 7.17-7.24 (m, 1H) 7.01-7.07 (m, 2H) 6.97 (d, J=1.79 Hz, 1H) 6.76-6.81 (m, 1H) 6.69-6.75 (m, 2H) 6.34-6.45 (m, 1H) 6.41 (s, 1H) 5.05 (dd, J=13.29, 5.07 Hz, 1H) 4.30-4.43 (m, 2H) 4.16-4.24 (m, 1H) 3.85 (s, 3H) 3.26 (br s, 4H) 3.07 (br t, J=12.04 Hz, 2H) 2.84-2.96 (m, 1H) 2.55-2.62 (m, 2H) 2.46 (br s, 4H) 2.31-2.39 (m, 1H) 1.87-2.29 (m, 8H) 1.53-1.67 (m, 3H) 1.02 (br t, J=12.22 Hz, 2H)

SFC: retention time, 1.996 min; Area, 94.495%.

### Example 24. Preparation of (1-156) (S)-3-(5-(4-((1-(4-((1R,2R)-4,4-difluoro-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione

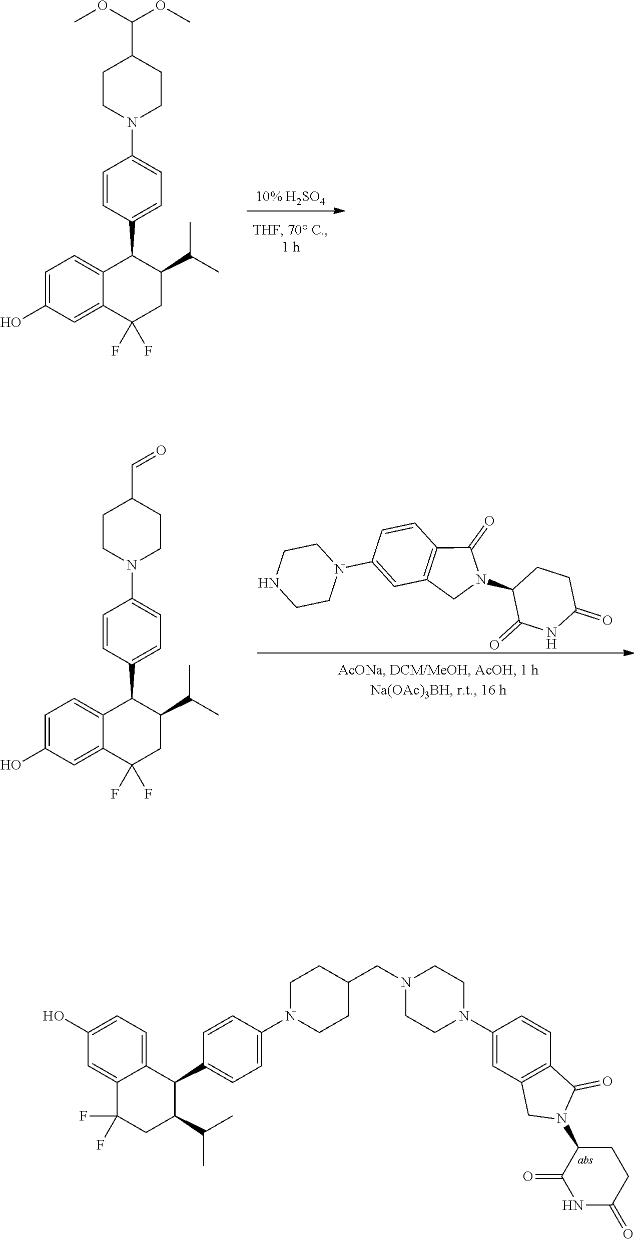 [SMILES: COC(OC)C1CCN(c2ccc([C@@H]3c4ccc(O)cc4C(F)(F)C[C@@H]3C(C)C)cc2)CC1 ; CC(C)[C@H]1CC(F)(F)c2cc(O)ccc2[C@H]1c1ccc(N2CCC(C=O)CC2)cc1 ; O=C1CC[C@H](N2Cc3cc(N4CCNCC4)ccc3C2=O)C(=O)N1 ; CC(C)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1]

Step 1: To a solution of (5R,6R)-5-(4-(4-(dimethoxymethyl)piperidin-1-yl)phenyl)-8,8-difluoro-6-isopropyl-5,6,7,8-tetrahydronaphthalen-2-ol (70 mg, 1 Eq, 0.15 mmol) in 10% H₂SO₄ (2 mL) and THF (2 mL) was stirred at 70° C. for 1 hour. TLC (petroleum ether:ethyl acetate=3:1, Rt=0.4, UV) showed a new spot was desired. The reaction was adjust to pH=˜8 with aq. NaHCO₃ (5 mL). Then the mixture was added water (30 mL) and extracted with ethyl acetate (2×30 mL).

The combined organic layers were washed with brine (30 mL), dried over anhydrous Na₂SO₄, filtered and concentrated to dryness in vacuo to give 1-(4-((1R,2R)-4,4-difluoro-6-hydroxy-2-isopropyl-1,2,3,4-tetrahydronaphthalen-1-yl)phenyl)piperidine-4-carbaldehyde (60 mg, 0.15 mmol, 95%) as a yellow solid.

Step 2: To a solution of 1-(4-((1R,2R)-4,4-difluoro-6-hydroxy-2-isopropyl-1,2,3,4-tetrahydronaphthalen-1-yl)phenyl)piperidine-4-carbaldehyde (60.0 mg, 1 Eq, 145 μmol), (S)-3-(1-oxo-5-(piperazin-1-yl)isoindolin-2-yl)piperidine-2,6-dione, PhSO₃H salt (109 mg, 1.5 Eq, 218 μmol) in DCM (4 mL) and MeOH (4 mL) was added sodium acetate (35.7 mg, 3 Eq, 435 μmol) was stirred at 25° C. for 60 mins. Then sodium triacetoxyborohydride (61.5 mg, 2 Eq, 290 μmol) and Acetic acid (26.1 mg, 25.0 μL, 3 Eq, 435 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um; mobile phase: [water(FA)-ACN]; B % 22%-52%, 7 min) to give (S)-3-(5-(4-((1-(4-((1R,2R)-4,4-difluoro-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione (20 mg, 27 μmol, 19%, 99.625% Purity) was obtained as a white solid. LC-MS (ESI+) m/z: 726.3 (M+H)⁺.

LCMS: calc. for C₄₂H₄₉F₂N₅O₄: 725.88, found: [M+H]⁺ 726.3.

HPLC: 99.625% purity at 220 nm.

¹H NMR (400 MHZ, DMSO-d₆) δ=10.96 (s, 1H), 8.18 (s, 0.672H), 7.52 (d, J=8.5 Hz, 1H), 7.11-7.02 (m, 2H), 6.99 (s, 1H), 6.89-6.73 (m, 6H), 5.05 (dd, J=5.0, 13.2 Hz, 1H), 4.40-4.27 (m, 1H), 4.26-4.14 (m, 2H), 3.62 (br d, J=10.5 Hz, 3H), 3.28 (br s, 5H), 2.97-2.83 (m, 1H), 2.64-2.53 (m, 4H), 2.46-2.24 (m, 3H), 2.21 (br d, J=7.0 Hz, 2H), 2.14-2.01 (m, 1H), 2.00-1.91 (m, 1H), 1.90-1.74 (m, 3H), 1.68 (br d, J=7.2 Hz, 1H), 1.33 (br dd, J=6.7, 13.4 Hz, 1H), 1.25-1.12 (m, 2H), 1.06 (d, J=6.4 Hz, 3H), 0.64 (d, J=6.7 Hz, 3H).

### Example 25. Preparation of (I-153) (S)-3-(5-(4-((1-(4-((1S,2S)-4,4-difluoro-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione

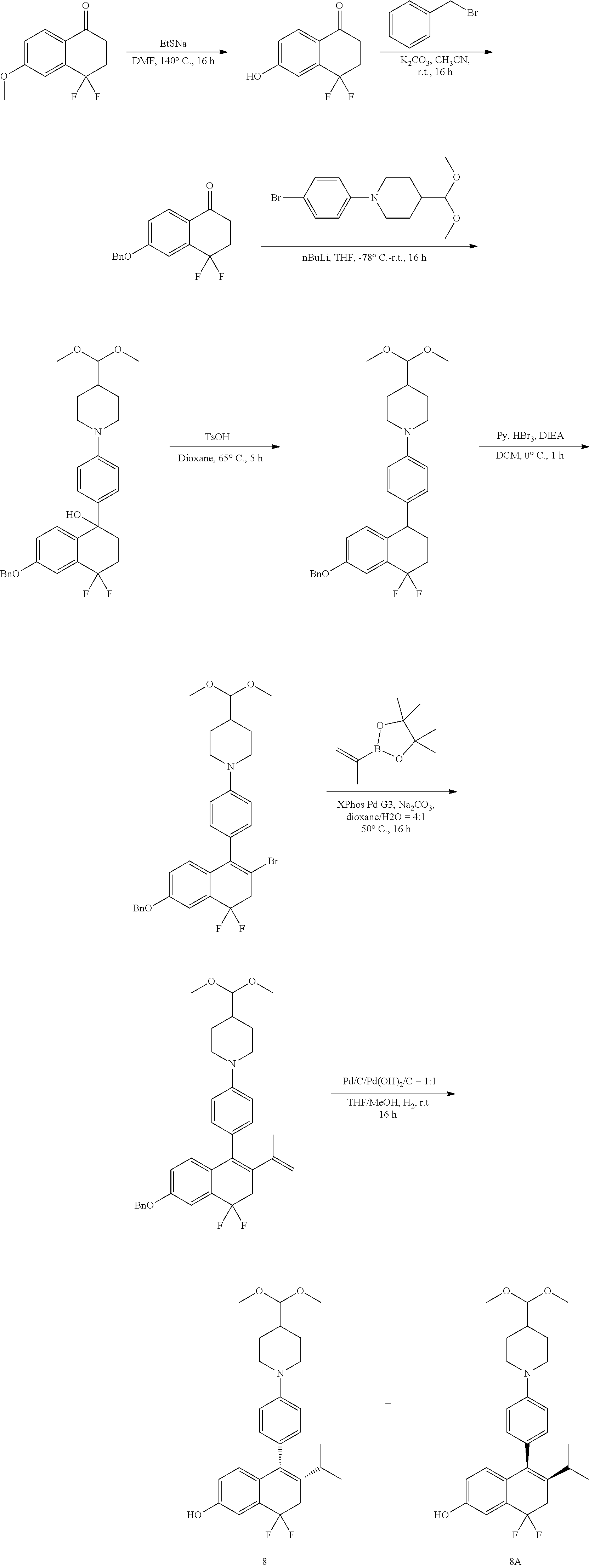

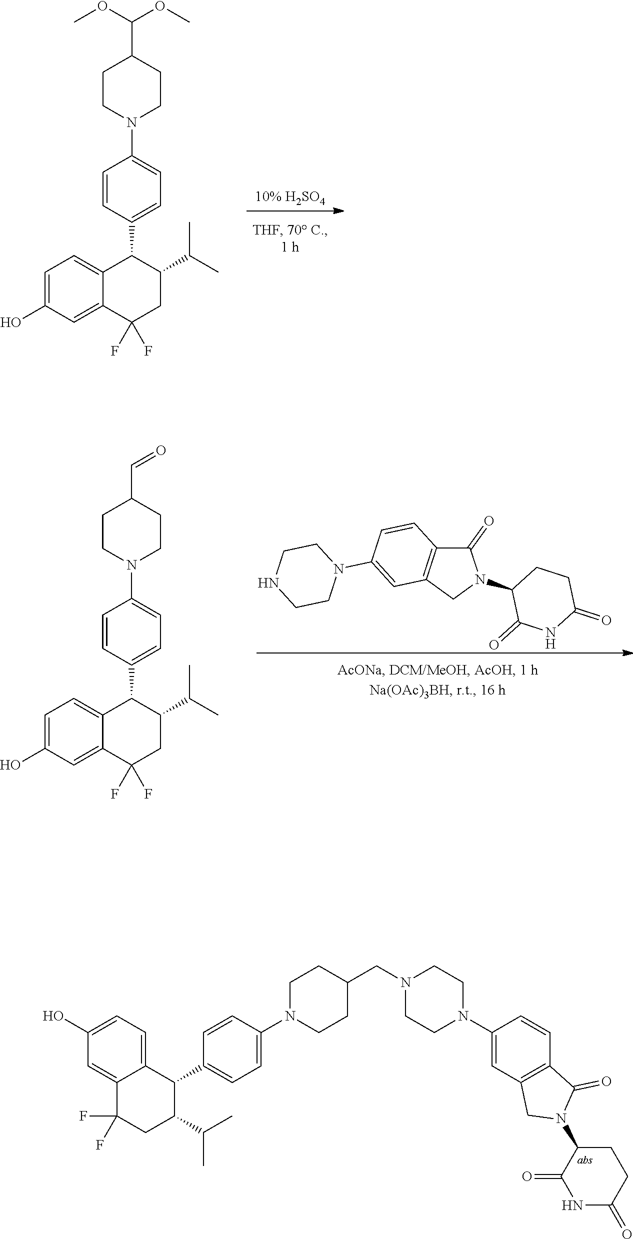 [SMILES: COC(OC)C1CCN(c2ccc([C@H]3c4ccc(O)cc4C(F)(F)C[C@H]3C(C)C)cc2)CC1 ; CC(C)[C@@H]1CC(F)(F)c2cc(O)ccc2[C@@H]1c1ccc(N2CCC(C=O)CC2)cc1 ; O=C1CC[C@H](N2Cc3cc(N4CCNCC4)ccc3C2=O)C(=O)N1 ; CC(C)[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1]

Step 1: To a solution of 4,4-difluoro-6-methoxy-3,4-dihydronaphthalen-1 (2H)-one (5.0 g, 1 Eq, 24 mmol) in DMF (100 mL) at r.t. was added sodiumethanethiolate (7.9 g, 4 Eq, 94 mmol) under N₂. After addition, the mixture was stirred at 140° C. for 16 h under N₂. The reaction was adjust to pH=6 with HCl (2 M, 50 mL) and added H₂O (200 mL) and extracted with ethyl acetate (300 mL×2). The combined organic layers were dried over Na₂SO₄, filtered and concentrated under reduced pressure to give a residue. The residue was purified by flash silica gel chromatography (ethyl acetate in petroleum ether=0% to 20%) to give the product of 4,4-difluoro-6-hydroxy-3,4-dihydronaphthalen-1 (2H)-one (4 g, 80% Yield, 90.159% Purity) as a brown solid, LC-MS (ESI⁺) m/z: 199.1 (M+H)⁺.

Step 2: To a solution of 4,4-difluoro-6-hydroxy-3,4-dihydronaphthalen-1 (2H)-one (3.8 g, 1 Eq, 17 mmol) in CH₃CN (50 mL) at r.t. was added (bromomethyl)benzene (3.5 g, 1.2 Eq, 21 mmol) and K₂CO₃ (4.8 g, 2 Eq, 35 mmol). After addition, the mixture was stirred at 25° C. for 16 h. The mixture was treated with H₂O (200 mL), extracted with EtOAc (200 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 6-(benzyloxy)-4,4-difluoro-3,4-dihydronaphthalen-1 (2H)-one (5.0 g, 100% yield, 100% Purity) as a white solid. LC-MS (ESI⁺) m/z: 289.1 (M+H)⁺.

Step 3: To a solution of 1-(4-bromophenyl)-4-(dimethoxymethyl)piperidine (6.5 g, 1.2 Eq, 21 mmol) in THF (60 mL) was added dropwise n-BuLi (1.4 g, 1.3 Eq, 23 mmol) at −78° C. under N₂. After addition, the mixture was stirred at this temperature for 1 h, and then 6-(benzyloxy)-4, 4-difluoro-3,4-dihydronaphthalen-1 (2H)-one (5.0 g, 1 Eq, 17 mmol) in THF (20 mL) was added dropwise at −78° C. The resulting mixture was stirred at −78° C. for 16 h. The reaction mixture was quenched with aq·NH₄Cl (50 mL) at 0° C., then added H₂O (200 mL) and extraction with EtOAc (300 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 6-(benzyloxy)-1-(4-(4-(dimethoxymethyl)piperidin-1-yl)phenyl)-4,4-difluoro-1,2,3,4-tetrahydronaphthalen-1-ol (7.3 g, 72% Yield, 89.103% Purity) as a yellow solid. LC-MS (ESI+) m/z: 524.3 (M+H)⁺.

Step 4: A mixture of 6-(benzyloxy)-1-(4-(4-(dimethoxymethyl)piperidin-1-yl)phenyl)-4,4-difluoro-1,2,3,4-tetrahydronaphthalen-1-ol (7.3 g, 1 Eq, 12 mmol) in Dioxane (70 mL) at r.t., then Ts-OH (0.24 g, 0.1 Eq, 1.2 mmol) was added to the mixture and was stirred at 65° C. for 5 h. The mixture was treated with H₂O (200 mL), extracted with EtOAc (300 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 11-(4-(6-(benzyloxy)-4,4-difluoro-3,4-dihydronaphthalen-1-yl)phenyl)-4-(dimethoxymethyl)piperidine (6.1 g, 63% Yield, 64.465% Purity) as a yellow solid. LC-MS (ESI⁺) m/z: 506.2 (M+H)⁺.

Step 5: To a mixture of 1-(4-(6-(benzyloxy)-4,4-difluoro-3,4-dihydronaphthalen-1-yl)phenyl)-4-(dimethoxymethyl)piperidine (1.5 g, 1 Eq, 1.9 mmol), DIEA (0.37 g, 1.5 Eq, 2.9 mmol) and Pyridinium bromide perbromide (0.92 g, 1.5 Eq, 2.9 mmol) in DCM (20 mL) at 0° C., then the mixture was stirred at 0° C. for 1 h. The mixture was treated with H₂O (150 mL), extracted with DCM (200 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-2-bromo-4,4-difluoro-3,4-dihydronaphthalen-1-yl)phenyl)-4-(dimethoxymethyl)piperidine (1.1 g, 68% Yield, 68.965% Purity) as a yellow solid. LC-MS (ESI+) m/z: 586.0 (M+H)⁺.

Step 6: XPhos Pd G3 (94.9 mg, 0.1 Eq, 112 μmol) was added to the mixture of 1-(4-(6-(benzyloxy)-2-bromo-4,4-difluoro-3,4-dihydronaphthalen-1-yl)phenyl)-4-(dimethoxymethyl)piperidine (950 mg, 1 Eq, 1.12 mmol), 4,4,5,5-tetramethyl-2-(prop-1-en-2-yl)-1,3,2-dioxaborolane (377 mg, 2 Eq, 2.24 mmol) and Sodium carbonate (356 mg, 3 Eq, 3.36 mmol) in Dioxane (10 mL) and H₂O (2.5 mL) at r.t. N₂ was bubbled into the mixture for 5 min. Then the reaction mixture was heated at 60° C. for 12 h. The mixture was added H₂O (150 mL) and extracted with EtOAc (150 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give a yellow solid. Then the yellow solid was purified by prep-HPLC (column: Xtimate C18 150*40 mm*10 um; mobile phase: [water(FA)-ACN]; B % 70%-100%, 6 min) to give 1-(4-(6-(benzyloxy)-4,4-difluoro-2-(prop-1-en-2-yl)-3,4-dihydronaphthalen-1-yl)phenyl)-4-(dimethoxymethyl)piperidine (320 mg, 52.3% Yield) as a white solid.

TLC (PE:EtOAc=2:1, Rf=0.5) confirmed.

Step 7: A mixture of 1-(4-(6-(benzyloxy)-4,4-difluoro-2-(prop-1-en-2-yl)-3,4-dihydronaphthalen-1-yl)phenyl)-4-(dimethoxymethyl)piperidine (300 mg, 1 Eq, 550 μmol), palladium (300 mg, 10% Wt, 0.513 Eq, 282 μmol), PdOH₂ (300 mg, 20% Wt, 0.777 Eq, 427 μmol) in MeOH (15 mL) and THF (15 mL) was degassed and purged with H₂ for 3 times, and then the mixture was stirred at 25° C. for 12 hour under H₂ atmosphere (15 psi). The reaction mixture was filtered and concentrated under reduced pressure to give a residue. The residue was concentrated under reduced pressure to give (5S,6S)-5-(4-(4-(dimethoxymethyl)piperidin-1-yl)phenyl)-8,8-difluoro-6-isopropyl-5,6,7,8-tetrahydronaphthalen-2-ol as a yellow oil, which was further separated by SFC (condition: column: DAICEL CHIRALPAK AD (250 mm*30 mm, 10 um); mobile phase: [0.1% NH, H₂O EtOH]; B %: 25%-25%) to give (5S,6S)-5-(4-(4-(dimethoxymethyl)piperidin-1-yl)phenyl)-8,8-difluoro-6-isopropyl-5,6,7,8-tetrahydronaphthalen-2-ol (50 mg, 0.11 mmol, 19%, 97.963% Purity) and (5R,6R)-5-(4-(4-(dimethoxymethyl)piperidin-1-yl)phenyl)-8,8-difluoro-6-isopropyl-5,6,7,8-tetrahydronaphthalen-2-ol (70 mg, 0.15 mmol, 28%, 100.0% Purity) as yellow solid.

Step 8: To a solution of (5S,6S)-5-(4-(4-(dimethoxymethyl)piperidin-1-yl)phenyl)-8,8-difluoro-6-isopropyl-5,6,7,8-tetrahydronaphthalen-2-ol (50 mg, 1 Eq, 0.11 mmol) in 10% H₂SO₄ (2 mL) and THF (2 mL) was stirred at 70° C. for 1 hour. TLC (petroleum ether:ethyl acetate=3:1, R_{f}=0.4UV) showed a new spot was desired. The reaction was adjust to PH˜=8 with aq. NaHCO₃ (5 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1S,2S)-4,4-difluoro-6-hydroxy-2-isopropyl-1,2,3,4-tetrahydronaphthalen-1-yl)phenyl)piperidine-4-carbaldehyde (60 mg, 0.15 mmol, 130%) as a yellow solid.

Step 9: To a solution of 1-(4-((1S,2S)-4,4-difluoro-6-hydroxy-2-isopropyl-1,2,3,4-tetrahydronaphthalen-1-yl)phenyl)piperidine-4-carbaldehyde (60.0 mg, 1 Eq, 145 μmol), (S)-3-(1-oxo-5-(piperazin-1-yl)isoindolin-2-yl)piperidine-2,6-dione, PhSO₃H salt (109 mg, 1.5 Eq, 218 μmol) in DCM (4 mL) and MeOH (4 mL) was added sodium acetate (35.7 mg, 3 Eq, 435 μmol) was stirred at 25° C. for 60 mins, Then Sodium triacetoxyborohydride (61.5 mg, 2 Eq, 290 μmol) and acetic acid (26.1 mg, 25.0 μL, 3 Eq, 435 μmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um; mobile phase: [water(FA)-ACN]; B % 22%-52%, 7 min) to give (S)-3-(5-(4-((1-(4-((1S,2S)-4,4-difluoro-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione (24.7 mg, 33.1 μmol, 22.8%, 97.332% Purity) was obtained as a white solid. LC-MS (ESI+) m/z: 726.3 (M+H)⁺.

LCMS: calc. for C₄₂H₄₉F₂N₅O₄: 725.88, found: [M+H]⁺ 726.3.

HPLC: 97.332% purity at 220 nm.

¹H NMR (400 MHZ, DMSO-d₆) δ=10.96 (s, 1H), 8.19 (s, 0.450H), 7.52 (d, J=8.6 Hz, 1H), 7.11-7.03 (m, 2H), 6.99 (s, 1H), 6.88-6.71 (m, 6H), 5.05 (dd, J=5.1, 13.3 Hz, 1H), 4.37-4.29 (m, 1H), 4.25-4.16 (m, 2H), 3.62 (br d, J=11.1 Hz, 3H), 3.29 (br s, 6H), 2.97-2.84 (m, 1H), 2.65-2.53 (m, 4H), 2.40-2.25 (m, 2H), 2.21 (br d, J=7.0 Hz, 2H), 2.17-2.02 (m, 1H), 2.01-1.92 (m, 1H), 1.90-1.74 (m, 3H), 1.66 (br d, J=6.0 Hz, 1H), 1.33 (dt, J=6.9, 13.9 Hz, 1H), 1.26-1.12 (m, 2H), 1.06 (d, J=6.4 Hz, 3H), 0.64 (d, J=6.7 Hz, 3H).

### Example 26. Preparation of (1-285) (R)-3-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione and (I-286) (S)-3-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione

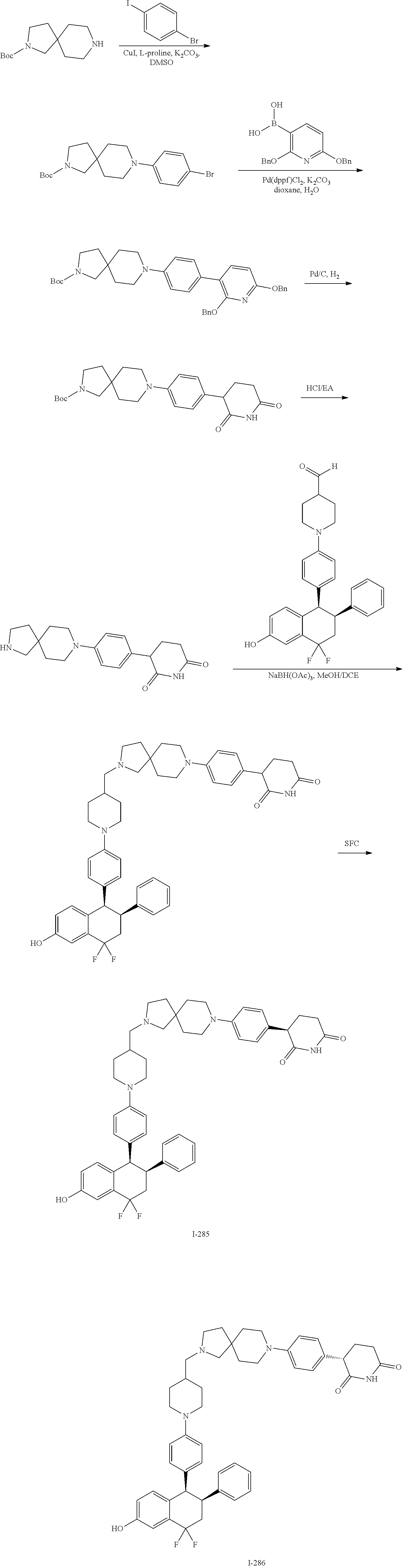

Step 1: A mixture of 1-bromo-4-iodo-benzene (600 mg, 2.12 mmol, 1 eq.), tert-butyl 2,8-diazaspiro[4.5]decane-2-carboxylate (611.68 mg, 2.55 mmol, 1.2 eq.), (28)-pyrrolidine-2-carboxylic acid (146.50 mg, 1.27 mmol, 0.6 eq.), iodocopper (242.35 mg, 1.27 mmol, 0.6 eq.) and K₂CO₃ (732.79 mg, 5.30 mmol, 2.5 eq.) in DMSO (3 mL) was degassed and purged with N₂ for 3 times, and then the mixture was stirred at 80° C. for 3 hour under N₂ atmosphere. LCMS showed one main peak with desired mass was detected. The reaction mixture was partitioned between EA (40 mL×2) and water (50 mL). After quenching the reaction, the reaction mixture was poured into separatory funnel and separated. The crude product was purified by silica gel chromatography eluted with PE:EtOAc=3:1. tert-butyl 8-(4-bromophenyl)-2,8-diazaspiro[4.5]decane-2-carboxylate (260 mg, 657.67 μmol, 31.01% yield) was obtained as a white solid.

LC-MS (ESI⁺) m/z: 397.1 (M+H)⁺.

Step 2: A mixture of tert-butyl 8-(4-bromophenyl)-2,8-diazaspiro[4.5]decane-2-carboxylate (100 mg, 253 μmol, 1.0 eq.), (2,6-bis(benzyloxy)pyridin-3-yl) boronic acid (93.3 mg, 278 μmol, 1.1 eq.), K₂CO₃ (105 mg, 759 μmol, 3.0 eq.) and PdCl₂(dppf) (18.5 mg, 25.3 μmol, 0.1 eq.) in Dioxane (2 mL) and Water (0.5 mL) was degassed and purged with N₂ for 3 times, and then the mixture was stirred at 80° C. for 3 hour under N₂ atmosphere. LCMS showed the reaction was completed. The reaction mixture was quenched by addition EA 10 mL at 20° C., and then diluted with water 10 mL and extracted with EA 30 mL (10 mL×3). The combined organic layers were washed with EA 15 mL (5 mL×3), dried over Na₂SO₄, filtered and concentrated under reduced pressure to give a residue. The crude material was purified on silica gel column chromatography (from pure PE to PE/EtOAc=4/1) to give tert-butyl 8-(4-(2,6-bis(benzyloxy)pyridin-3-yl)phenyl)-2,8-diazaspiro[4.5]decane-2-carboxylate (120 mg, 0.16 mmol, 63% yield, 80% Purity) as a yellow solid.

LC-MS (ESI+) m/z: 606.3 (M+H)⁺.

Step 3: To a solution of tert-butyl 8-(4-(2,6-bis(benzyloxy)pyridin-3-yl)phenyl)-2,8-diazaspiro[4.5]decane-2-carboxylate (0.10 g, 0.17 mmol, 1.0 eq.) in EtOH (3 mL), DCM (3 mL) and EtOAc (3 mL) was added Pd/C (5 wt %, 0.1 g) under H₂ atmosphere. The suspension was degassed and purged with H₂ for 3 times. The mixture was stirred under H₂ (30 psi) at 30° C. for 16 hour. LCMS showed the reaction was completed. The mixture was filtered and the filtrate was concentrated to give tert-butyl 8-(4-(2,6-dioxopiperidin-3-yl)phenyl)-2,8-diazaspiro[4.5]decane-2-carboxylate (65 mg, 0.15 mmol, 92%) as a white solid.

LC-MS (ESI⁺) m/z: 428.2 (M+H)⁺.

Step 4: To a solution of tert-butyl 8-(4-(2,6-dioxopiperidin-3-yl)phenyl)-2,8-diazaspiro[4.5]decane-2-carboxylate (60 mg, 0.14 mmol, 1.0 eq.) in HCL/MeOH (3 mL). The mixture was stirred at 20° C. for 1 hour. LCMS showed the reaction was completed. The mixture was concentrated in vacuum to give crude product. No further purification as it is used for the next step directly.

LC-MS (ESI+) m/z: 328.2 (M+H)⁺+.

Step 5: To a solution of 3-(4-(2,8-diazaspiro[4.5]decan-8-yl)phenyl)piperidine-2,6-dione (140.0 mg, 427.6 μmol, 2.0 eq.) in MeOH (6 mL) and DCE (9 ml) was added 1-(4-((1R,2S)-4,4-difluoro-6-hydroxy-2-phenyl-1,2,3,4-tetrahydronaphthalen-1-yl)phenyl)piperidine-4-carbaldehyde (95.68 mg, 213.8 μmol, 1.0 eq.) at 25° C. After addition, the mixture was stirred at this temperature for 0.5 hr, and then NaBH₃(OAc)₃ (271.9 mg, 190 μL, 3 Eq, 1.283 mmol)) was added at 25° C. The resulting mixture was stirred at 25° C. for 3 br. The reaction mixture was concentrated to yield a residue. The residue was purified by preparative HPLC (column: Boston Green ODS 150*30 mm*5 um; mobile phase: [water(FA)-ACN]; B %: 15%-45%, 12 min), followed by lyophilization to yield 3-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (120 mg, 158 μmol, 37.0%) was obtained as a white solid. LC-MS (ESH) m/z: 759.3 [M+H]⁺.

Step 6:3-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (120 mg, 158 μmol, 1.0 eq) was separated by chiral SFC(Column DAICEL CHIRALPAK ID (250 mm*30 mm, 10 um), Condition MeOH-ACN Begin B 50% End B 50%) FlowRate (ml/min): 80).

I-285 (37.1 mg, 48.9 μmol, 30.9%). LC-MS (ESI+) m/z: 759.4 (M+H)⁺.

LCMS: calc. for C₄₇H₅₂F₂N₄O₃: 758.95, found: [M+H]⁺ 759.4.

HPLC: 100% purity at 220 nm

¹H NMR (400 MHz, DMSO-d₆) δ ppm 10.77 (s, 1H) 9.73 (s, 1H) 7.17-7.22 (m, 3H) 7.08 (s, 1H) 7.02 (d, J=8.70 Hz, 2H) 6.88-6.91 (m, 3H) 6.83 (br d, J=0.95 Hz, 3H) 6.55 (d, J=8.70 Hz, 2H) 6.12 (d, J=8.58 Hz, 2H) 4.29 (br d, J=2.74 Hz, 1H) 3.63-3.74 (m, 2H) 3.47-3.54 (m, 2H) 3.03-3.14 (m, 4H) 2.58-2.69 (m, 2H) 2.42-2.48 (m, 4H) 2.27-2.34 (m, 3H) 2.21 (br d, J=7.03 Hz, 2H) 2.11 (td, J=11.59, 8.29 Hz, 1H) 1.96-2.03 (m, 1H) 1.70-1.77 (m, 2H) 1.43-1.65 (m, 8H) 1.06-1.16 (m, 2H).

I-286 (39.5 mg, 52.0 μmol, 32.9%). LC-MS (ESI+) m/z: 759.4 (M+H)⁺.

LCMS: calc. for C₄₇H₅₂F₂N₄O₃: 758.95, found: [M+H]⁺ 759.4.

HPLC: 100% purity at 220 nm

¹H NMR (400 MHZ, DMSO-d₆) δ ppm 10.78 (s, 1H) 9.74 (s, 1H) 7.17-7.23 (m, 3H) 7.08 (s, 1H) 7.03 (d, J=8.70 Hz, 2H) 6.89-6.92 (m, 3H) 6.83-6.88 (m, 3H) 6.56 (d, J=8.70 Hz, 2H) 6.12 (d, J=8.70 Hz, 2H) 4.30 (br d, J=3.10 Hz, 1H) 3.64-3.74 (m, 2H) 3.49-3.56 (m, 2H) 3.04-3.16 (m, 4H) 2.57-2.68 (m, 2H) 2.41-2.49 (m, 4H) 2.27-2.36 (m, 3H) 2.22 (br d, J=6.32 Hz, 2H) 2.07-2.15 (m, 1H) 1.97-2.04 (m, 1H) 1.71-1.78 (m, 2H) 1.38-1.68 (m, 8H) 1.07-1.17 (m, 2H).

### Example 27. Preparation of (1-317) (S)-3-(5-(4-((1-(4-((1S,2R)-2-(2,5-difluorophenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione and (I-316) (S)-3-(5-(4-((1-(4-((1R,2S)-2-(2,5-difluorophenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

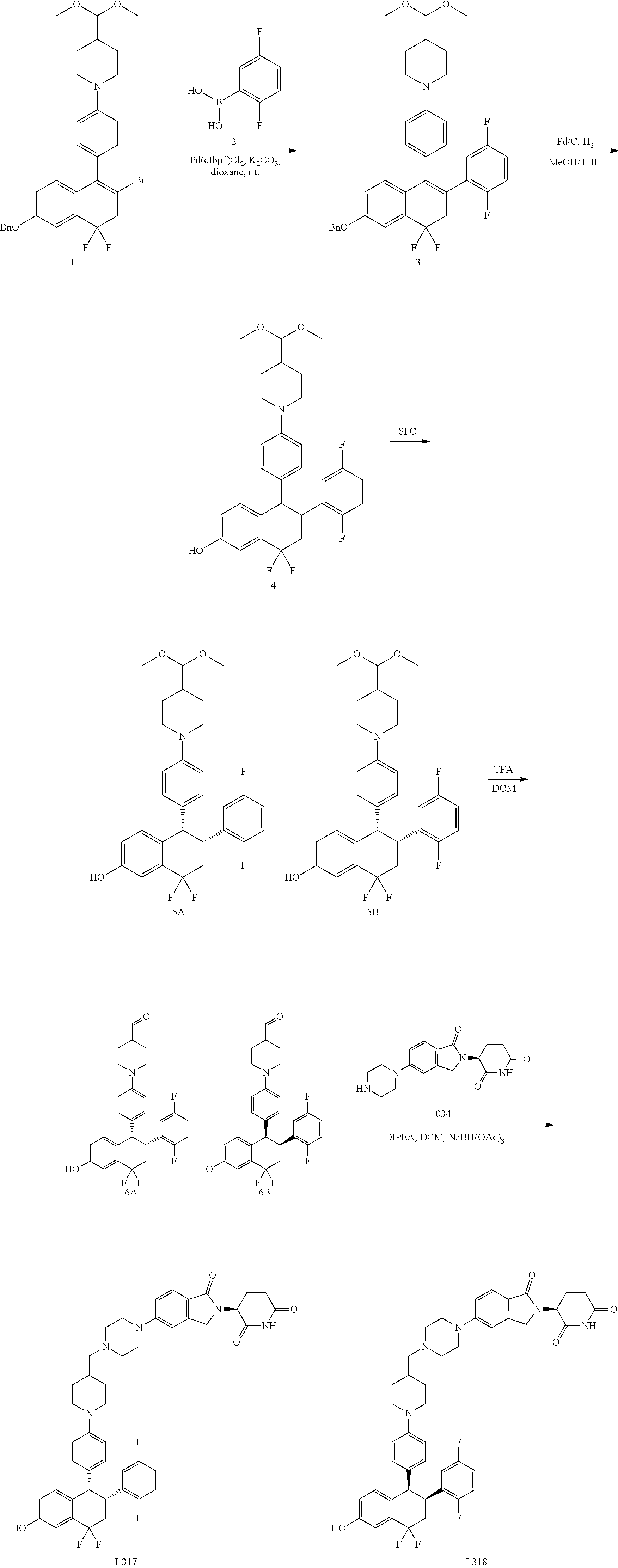

Step 1: To a mixture of compound 1 (604 mg, 1.03 mmol, 1.0 eq) and compound 2 (163.18 mg, 1.03 mmol, 1.0 eq) in 1, 4-Dioxane (8 mL) and water (2 mL) was added Pd(dtbpf)Cl₂ (67.35 mg, 103.34 μmol, 0.1 eq), K₂CO₃ (285.65 mg, 2.07 mmol, 2.0 eq) in one portion at 25° C. under N₂. The mixture was stirred for 0.5 hours. The reaction mixture extracted with EtOAc (20 mL×3). The combined organic layers were washed with solvent 10 mL saturated NaCl solution, dried over Na₂SO₄, filtered and concentrated under reduced pressure to give a residue. The residue was purified by prep-HPLC (column: Boston Green ODS 150*30 mm*5 um; mobile phase: water (FA)-ACN; B %: 80%-100%, 12 min) to give product. Compound 3 (200 mg, 323.80 μmol, 31.33% yield) was obtained as a white solid.

LC-MS (ESI⁺) m/z: 618.4 (M+H)⁺.

Step 2: To a solution of compound 3 (250 mg, 404.75 μmol, 1.0 eq) in THF (4 mL) and MeOH (4 mL) was added Pd/C (215.37 mg, 202.37 μmol, 10 wt %, 0.5 eq). The suspension was degassed under vacuum and purged with H₂ several times. The mixture was heated to 50° C. (15 psi) and stirred for 6 hours. LCMS showed that the desired compound was detected. The crude product was filtered and the filter was concentrated to give the crude as a yellow solid. The mixture was filtered and concentrated in vacuum. The residue was purified by pre-HPLC (Column Boston Green ODS 150*30 mm*5 um; Condition water(FA)-ACN; B %: 10%-40%, 12 min) to afford compound 4 (110.3 mg, 208.28 μmol, 51.46% yield) as a white solid.

LC-MS (ESI⁺) m/z: 530.3 (M+H)⁺.

Step 3: Compound 4 (100 mg) was separated by chiral SFC(Column DAICEL CHIRALPAK AD (250 mm*30 mm, 10 um), Condition CO₂-EtOH (0.1% NH₃H₂O) Begin B 20% End B 20%) FlowRate (ml/min): 60) Compound 5A (52 mg), compound 5B (41 mg).

Step 4: Compound 6A: To a solution of compound 5A (52 mg, 98.19 μmol, 1.0 eq) in DCM (4 mL) was added TFA (11.20 mg, 98.19 μmol, 1.0 eq.). The mixture was stirred at 25° C. for 0.2 hr. LCMS showed that the starting material was consumed and the desired product was detected. The reaction liquid is dried with N₂ at 0° C. to give Compound 6A (40.2 mg, 83.14 μmol, 84.67% yield) was obtained as yellow oil which was used in the next step without further purification.

LC-MS (ESI+) m/z: 484.3 (M+H)⁺.

Step 5: Compound 6B: To a solution of compound 5B (41 mg, 77.42 μmol, 1.0 eq) in DCM (4 mL) was added TFA (8.83 mg, 77.42 μmol, 1.0 eq). The mixture was stirred at 25° C. for 0.2 hr. LCMS showed that the starting material was consumed and the desired product was detected. The reaction liquid is dried with N₂ at 0° C. to give Compound 6B (35.6 mg, 73.63 μmol, 95.10% yield) was obtained as yellow oil which was used in the next step without further purification.

LC-MS (ESI+) m/z: 484.3 (M+H)⁺.

Step 6: To a solution of compound 6A (40.2 mg, 83.14 μmol, 1.0 eq) in DCM (4 mL) and MeOH (4 mL) was added compound 034 (27.30 mg, 83.14 μmol, 1.0 eq) and DIEA (10.75 mg, 83.14 μmol, 1.0 eq) at 25° C. After addition, the mixture was stirred at this temperature for 0.5 hr, and then NaBH(OAc); (35.24 mg, 166.29 μmol, 2.0 eq) was added at 25° C. The resulting mixture was stirred at 25° C. for 1 hr. The reaction mixture was concentrated to yield a residue. The residue was purified by preparative HPLC (column: C18-1 150*30 mm*5 um; mobile phase: [water (NH₄HCO₃)-ACN]; B %: 52%-80%, 11 min), followed by lyophilization to yield 1-317 (27.7 mg, 34.81 μmol, 41.86% yield) was obtained as a white solid. LC-MS (ESI+) m/z: 796.5 [M+H]⁺.

LCMS: calc. for C₄₅H₄₅F₄N₅O₄: 795.34, found: [M+H]⁺ 796.5.

HPLC: 100% purity at 220 nm.

¹H NMR (400 MHz, DMSO-d₆) δ ppm 10.91 (s, 1H), 9.74 (s, 1H), 7.49 (d, J=8.70 Hz, 1H), 7.23 (dt, J=9.36, 4.74 Hz, 1H), 7.02-7.09 (m, 4H), 6.86 (br d, J=10.01 Hz, 2H), 6.61 (d, J=8.70 Hz, 2H), 6.33 (ddd, J=9.09, 5.87, 3.16 Hz, 1H), 6.20 (d, J=8.58 Hz, 2H), 5.02 (dd, J=13.17, 4.95 Hz, 1H), 4.32 (s, 4H), 3.77-3.84 (m, 1H), 3.54 (br d, J=12.04 Hz, 2H), 3.25-3.28 (m, 6H), 2.83-2.92 (m, 1H), 2.56-2.67 (m, 2H), 2.24-2.43 (m, 4H), 2.16 (br d, J=6.79 Hz, 2H), 1.91-1.97 (m, 1H), 1.54-1.85 (m, 4H), 1.10-1.17 (m, 2H).

Step 7: To a solution of compound 6A (30 mg, 62.05 μmol, 1.0 eq) in DCM (4 mL) and MeOH (4 mL) was added compound 034 (20.37 mg, 62.05 μmol, 1.0 eq) and DIEA (8.02 mg, 62.05 μmol, 1.0 eq) at 25° C. After addition, the mixture was stirred at this temperature for 0.5 hr, and then NaBH(OAc)₃ (26.30 mg, 124.10 μmol, 2.0 eq) was added at 25° C. The resulting mixture was stirred at 25° C. for 1 hr. The reaction mixture was concentrated to yield a residue. The residue was purified by preparative HPLC (column: C18-1 150*30 mm*5 um; mobile phase: [water (NH₄HCO₃)-ACN]; B %: 52%-80%, 11 min), followed by lyophilization to yield I-316 (38.7 mg, 48.63 μmol, 78.37% yield) was obtained as a white solid. LC-MS (ESI+) m/z: 796.6 [M+H]⁺.

LCMS: calc. for C₄₅H₄₅F₄N₅O₄: 795.34, found: [M+H]⁺ 796.6.

HPLC: 100% purity at 220 nm

¹H NMR (400 MHZ, DMSO-d₆) δ ppm 10.94 (s, 1H), 9.77 (s, 1H), 7.52 (d, J=8.70 Hz, 1H), 7.24-7.29 (m, 1H), 7.04-7.11 (m, 4H), 6.89 (br d, J=10.13 Hz, 2H), 6.64 (d, J=8.70 Hz, 2H), 6.36 (ddd, J=9.09, 5.75, 3.28 Hz, 1H), 6.23 (d, J=8.58 Hz, 2H), 5.05 (dd, J=13.17, 5.07 Hz, 1H), 4.12 (s, 4H), 3.81-3.87 (m, 1H), 3.54-3.59 (m, 2H), 3.28-3.30 (m, 6H), 2.85-2.92 (m, 1H), 2.60-2.69 (m, 2H), 2.26-2.46 (m, 4H), 2.19 (br d, J=6.91 Hz, 2H), 1.94-1.99 (m, 1H), 1.54-1.83 (m, 4H), 1.16 (br d, J=11.09 Hz, 2H).

### Example 28. Preparation of (I-326) (S)-3-(5-(4-((1-(4-((1R,2S)-4,4-difluoro-2-(4-fluoro-2-methylphenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

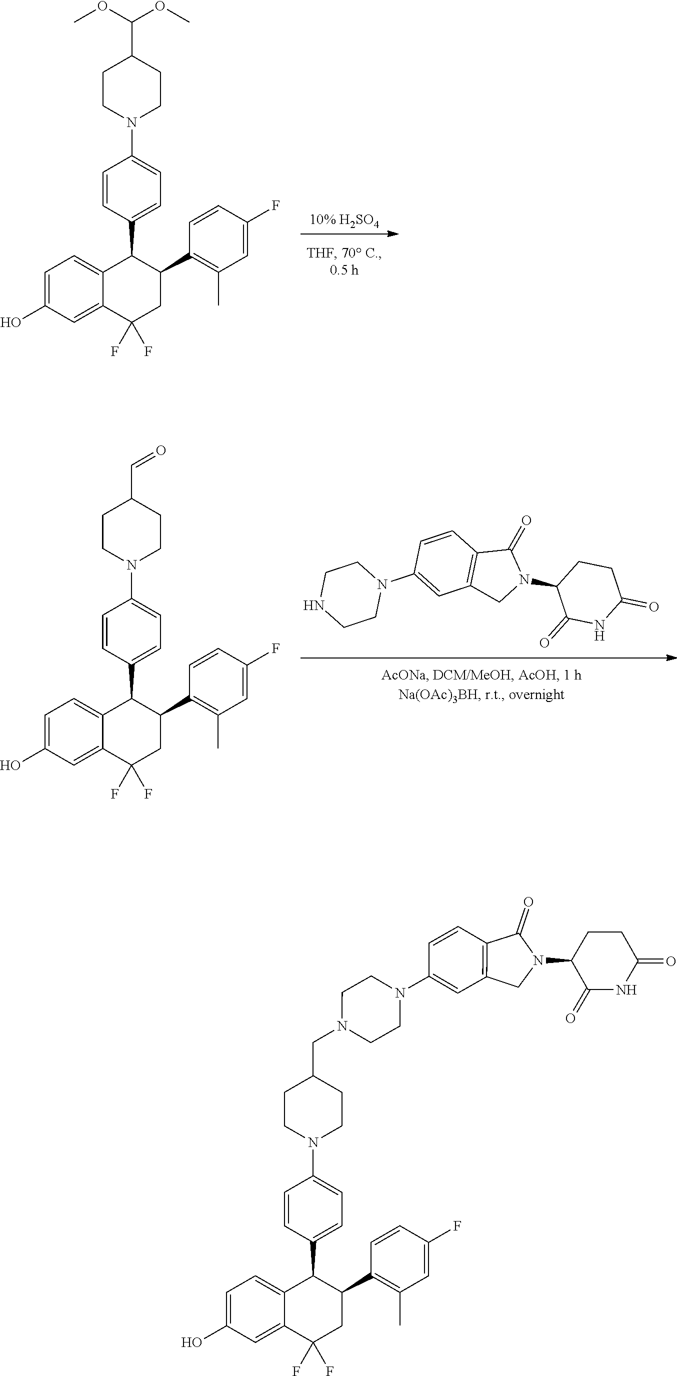 [SMILES: COC(OC)C1CCN(c2ccc([C@@H]3c4ccc(O)cc4C(F)(F)C[C@@H]3c3ccc(F)cc3C)cc2)CC1 ; Cc1cc(F)ccc1[C@H]1CC(F)(F)c2cc(O)ccc2[C@H]1c1ccc(N2CCC(C=O)CC2)cc1 ; O=C1CC[C@H](N2Cc3cc(N4CCNCC4)ccc3C2=O)C(=O)N1 ; Cc1cc(F)ccc1[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1]

Step 1: A mixture of (5R,6S)-5-(4-(4-(dimethoxymethyl)piperidin-1-yl)phenyl)-8,8-difluoro-6-(4-fluoro-2-methylphenyl)-5,6,7,8-tetrahydronaphthalen-2-ol (65 mg, 1 Eq, 0.12 mmol) in THF (1.5 mL), then 10% H₂SO₄ (1.5 mL) was added to the mixture and stirred at 70° C. for 0.5 h. The reaction was adjust to pH=8 with aq. NaHCO₃ at 0° C. Then the mixture was added H₂O (10 mL) and extracted with EtOAc (20 mL×2). The combined organic layers were washed with brine (20 mL), dried over anhydrous Na₂SO₄, filtered and concentrated to dryness in vacuo to give 1-(4-((1R,2S)-4,4-difluoro-2-(4-fluoro-2-methylphenyl)-6-hydroxy-1,2,3,4-tetrahydronaphthalen-1-yl)phenyl)piperidine-4-carbaldehyde (65 mg, 100% yield, 91.7% purity) as a yellow solid. LC-MS (ESI+) m/z: 480.3 (M+H)⁺.

Step 2: A mixture of 1-(4-((1R,2S)-4,4-difluoro-2-(4-fluoro-2-methylphenyl)-6-hydroxy-1,2,3,4-tetrahydronaphthalen-1-yl)phenyl)piperidine-4-carbaldehyde (65 mg, 1 Eq, 0.12 mmol), (S)-3-(1-oxo-5-(piperazin-1-yl)isoindolin-2-yl)piperidine-2,6-dione benzenesulfonate (79 mg, 1.3 Eq, 0.16 mmol), sodium acetate (51 mg, 5 Eq, 0.62 mmol) and acetic acid (22 mg, 21 μL, 3 Eq, 0.37 mmol) in DCM (3 mL) and MeOH (3 mL) was stirred at 25° C. for 1 h, then NaBH(OAc)₃ (53 mg, 2 Eq, 0.25 mmol) was added to the mixture and was stirred at 25° C. for 16 h. The mixture was added H₂O (20 mL), extracted with EtOAc (20 mL×2). The combined extracts was dried over anhydrous Na₂SO₄, filtered and concentrated to dryness in vacuo to give a residue. The residue was purified by prep-HPLC (column: Welch Xtimate C18 150*30 mm*5 um; mobile phase: [water(FA)-ACN]; B % 25%-65%, 9 min) to give (S)-3-(5-(4-((1-(4-((1R,2S)-4,4-difluoro-2-(4-fluoro-2-methylphenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (16.1 mg, 16% yield, 100% purity) as a white solid. LC-MS (ESI+) m/z: 792.3 (M+H)⁺.

LCMS: calc. for C₄₆H₄₈F3N5O4: 791.37, found: [M+H]+ 792.3.

HPLC: 100% purity at 220 nm.

¹HNMR (400 MHz, DMSO-d₆) δ=10.95 (s, 1H), 9.75 (s, 1H), 8.14 (s, 1H), 7.55 (br s, 1H), 7.21-7.03 (m, 4H), 6.95-6.83 (m, 2H), 6.73-6.57 (m, 3H), 6.20-6.10 (m, 3H), 5.06 (br dd, J=4.9, 13.4 Hz, 1H), 4.40-4.18 (m, 3H), 3.98 (br s, 1H), 3.72 (br d, J=15.4 Hz, 1H), 3.57 (br s, 3H), 3.31-3.24 (m, 3H), 3.11 (br s, 2H), 2.96-2.86 (m, 1H), 2.79-2.53 (m, 6H), 2.47 (s, 3H), 2.44-2.31 (m, 2H), 2.22 (br t, J=14.9 Hz, 2H), 2.02-1.92 (m, 1H), 1.80 (br s, 2H), 1.24 (br s, 2H)

### Example 29. Preparation of (I-406) (S)-3-(5-(4-((1-(4-((1R,2R)-4,4-difluoro-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione

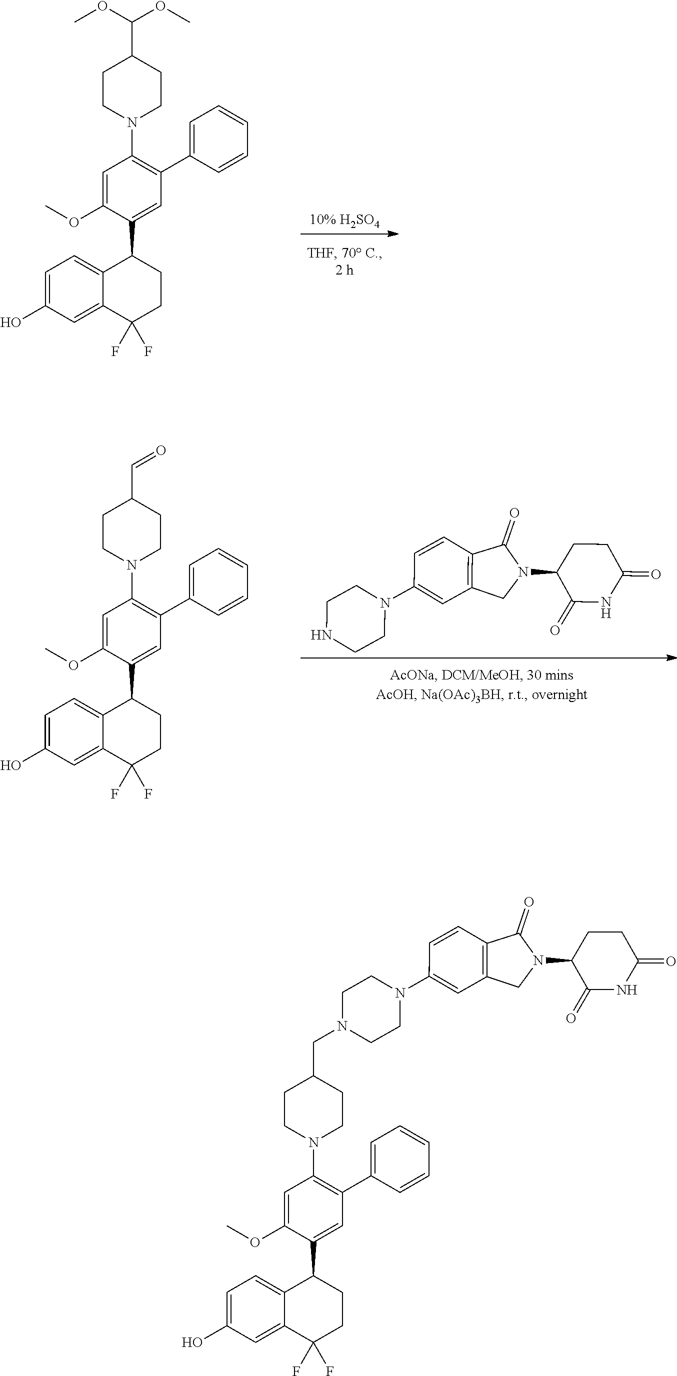 [SMILES: COc1cc(N2CCC(C(OC)OC)CC2)c(-c2ccccc2)cc1[C@H]1CCC(F)(F)c2cc(O)ccc21 ; COc1cc(N2CCC(C=O)CC2)c(-c2ccccc2)cc1[C@H]1CCC(F)(F)c2cc(O)ccc21 ; O=C1CC[C@H](N2Cc3cc(N4CCNCC4)ccc3C2=O)C(=O)N1 ; COc1cc(N2CCC(CN3CCN(c4ccc5c(c4)CN([C@H]4CCC(=O)NC4=O)C5=O)CC3)CC2)c(-c2ccccc2)cc1[C@H]1CCC(F)(F)c2cc(O)ccc21]

Step 1: To a solution of (S)-5-(6-(4-(dimethoxymethyl)piperidin-1-yl)-4-methoxy-[1,1′-biphenyl]-3-yl)-8,8-difluoro-5,6,7,8-tetrahydronaphthalen-2-ol (80.00 mg, 1 Eq, 152.8 μmol) in THF (3 mL) and 10% H₂SO₄ (3 mL). The mixture was stirred at 70° C. for 2 hour. LCMS showed 93% desired MS was detected. The reaction mixture was quenched by addition 50 mL H₂O at 25° C., and then diluted with 50 mL H₂O and extracted with ethyl acetate (50 mL*2). The combined organic layers were washed with 50 mL brine and dried over Na₂SO₄, filtered and concentrated under reduced pressure to give (S)-1-(5-(4,4-difluoro-6-hydroxy-1,2,3,4-tetrahydronaphthalen-1-yl)-4-methoxy-[1,1′-biphenyl]-2-yl)piperidine-4-carbaldehyde (70 mg, 0.14 mmol, 89%, 93% purity) as a residue. LC-MS (ESI+) m/z: 478.1 (M+H)⁺.

Step 2: To a solution of (S)-1-(5-(4,4-difluoro-6-hydroxy-1,2,3,4-tetrahydronaphthalen-1-yl)-4-methoxy-[1, l′-biphenyl]-2-yl)piperidine-4-carbaldehyde (70.00 mg, 1 Eq, 146.6 μmol), (S)-3-(1-oxo-5-(piperazin-1-yl)isoindolin-2-yl)piperidine-2,6-dione benzenesulfonate (85.58 mg, 1.2 Eq, 175.9 μmol) in DCM (2 mL), MeOH (2 mL) was added sodium acetate (36.07 mg, 3 Eq, 439.7 μmol) was stirred at 25° C. for 30 mins, Then sodium triacetoxyborohydride (62.13 mg, 2 Eq. 293.2 μmol) and acetic acid (26.41 mg, 25.29 μL, 3 Eq, 439.7 μmol) was added. The mixture was stirred at 25° C. for 16 hour. LCMS showed 93% desired MS was detected. The reaction mixture was concentrated under reduced pressure to remove solvent. The residue was purified by prep-HPLC (column: Welch Xtimate C18 150*30 mm*5 um phase: [water(FA)-ACN]; B %: 17%-57%, 9 min to give (S)-3-(5-(4-((1-(4-((1R,2R)-4,4-difluoro-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione (28.2 mg, 34.8 μmol, 23.7%, 97.4% purity) was obtained as a white solid.

LCMS: calc. for C₄₆H₄₉F₂N₅O₅: 789.92, found: [M+H]⁺ 790.4.

HPLC: 97.472% purity at 220 nm.

¹H NMR (400 MHz, DMSO-d₆) δ=10.96 (s, 1H) 9.20-10.38 (m, 1H) 6.17 (s, 0.586H) 7.52 (d, J=8.53 Hz, 1H) 7.39-7.45 (m, 2H) 7.33 (t, J=7.53 Hz, 2H) 7.17-7.25 (m, 1H) 7.01-7.08 (m, 2H) 6.97 (d, J=1.76 Hz, 1H) 6.75-6.81 (m, 1H) 6.67-6.75 (m, 2H) 6.41 (s, 1H) 5.05 (dd, J=13.30, 5.02 Hz, 1H) 4.28-4.43 (m, 2H) 4.15-4.24 (m, 1H) 3.85 (s, 3H) 3.25 (br s, 4H) 3.07 (br t, J=11.67 Hz, 2H) 2.84-2.97 (m, 1H) 2.53-2.62 (m, 2H) 2.46 (br s, 4H) 2.31-2.40 (m, 1H) 1.88-2.28 (m, 8H) 1.52-1.67 (m, 3H) 0.96-1.10 (m, 2H)

### Example 30. Preparation of (I-320) (S)-3-(5-(4-((1-(4-((1*S, 2*R)-4,4-difluoro-6-hydroxy-2-(p-tolyl)-1,2,3,4-tetrahydronaphthalen-1-yl)phenyl)piperidin-4-yl)methyl)piperazin-1-yl)-1-oxoisoindolin-2-yl)piperidine-2,6-dione

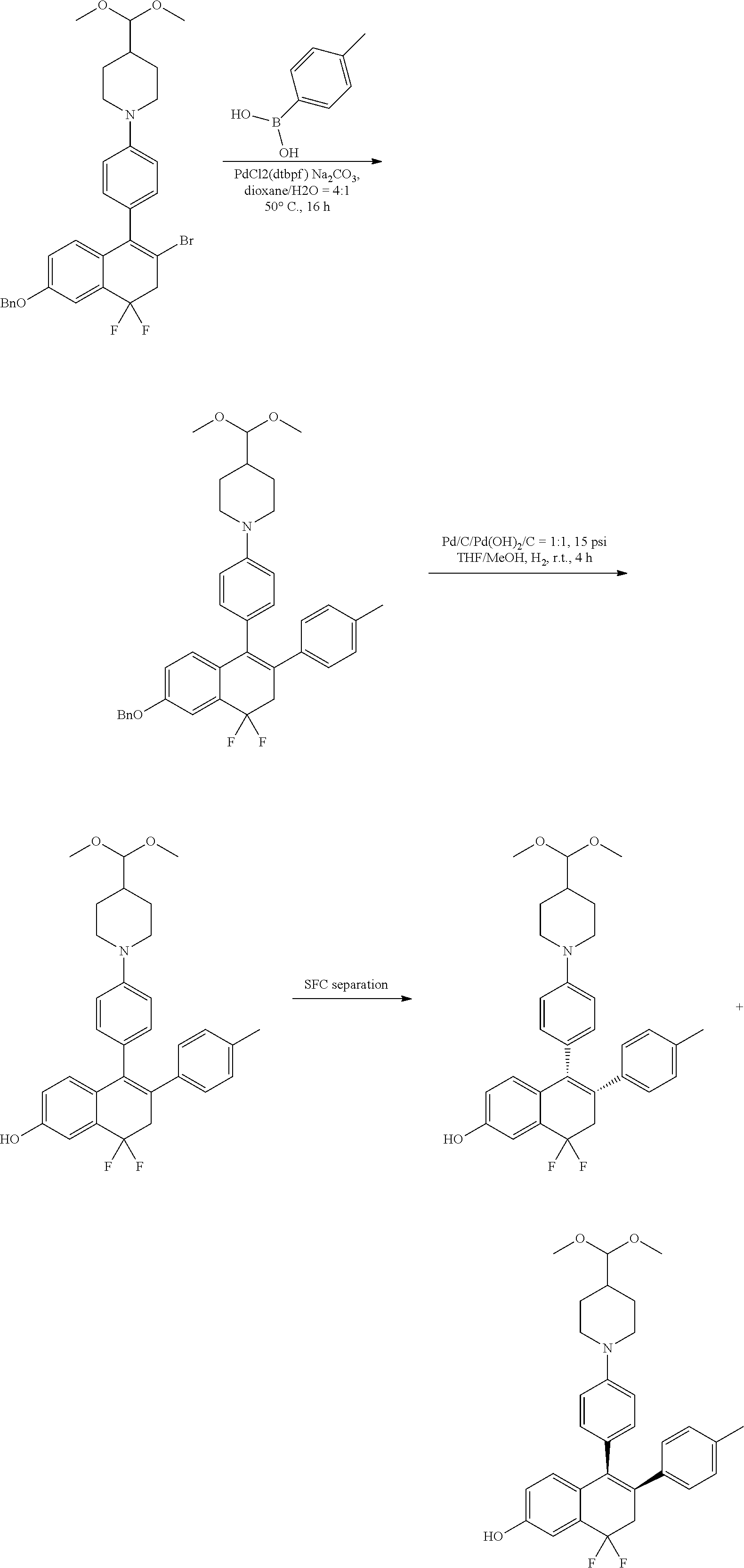

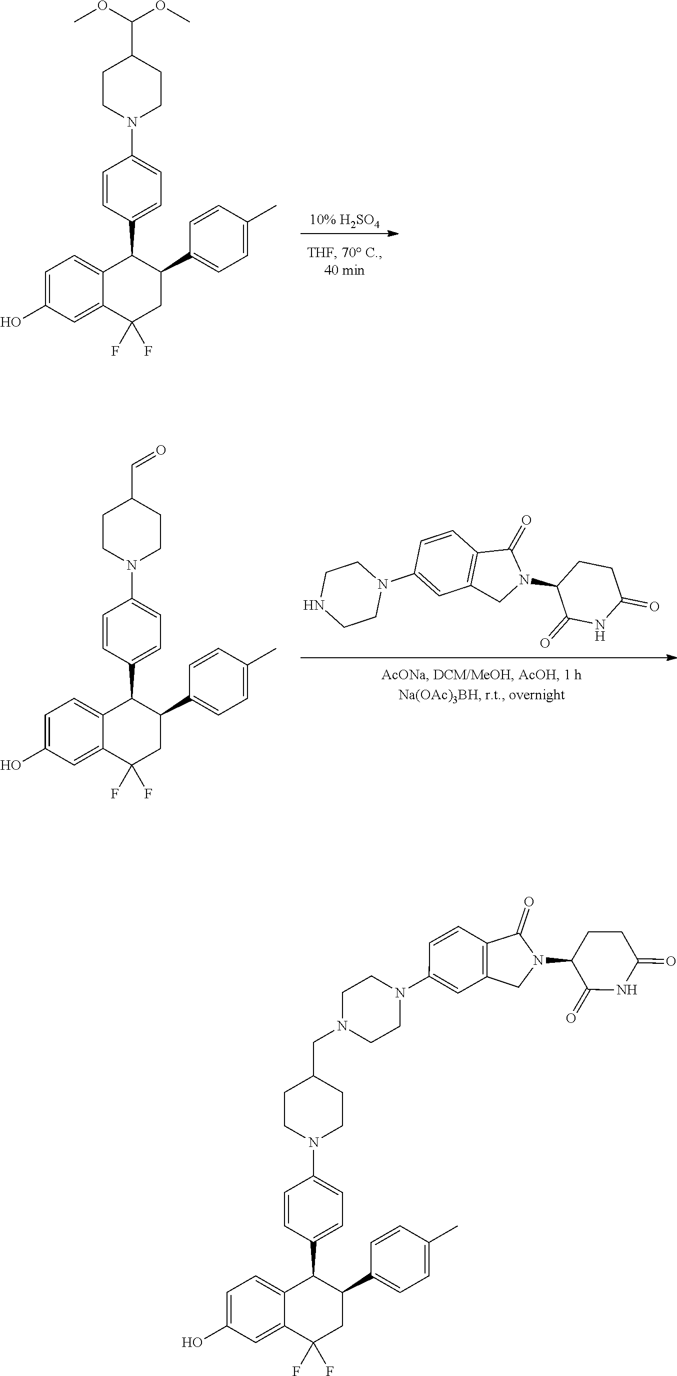 [SMILES: COC(OC)C1CCN(c2ccc([C@@H]3c4ccc(O)cc4C(F)(F)C[C@@H]3c3ccc(C)cc3)cc2)CC1 ; Cc1ccc([C@H]2CC(F)(F)c3cc(O)ccc3[C@H]2c2ccc(N3CCC(C=O)CC3)cc2)cc1 ; O=C1CC[C@H](N2Cc3cc(N4CCNCC4)ccc3C2=O)C(=O)N1 ; Cc1ccc([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)cc1]

Step 1: A 40 mL thread vial equipped with magnetic stirrer. To a mixture of 1-(4-(6-(benzyloxy)-2-bromo-4,4-difluoro-3,4-dihydronaphthalen-1-yl)phenyl)-4-(dimethoxymethyl)piperidine (600 mg, 1 Eq, 901 μmol), p-Tolueneboronic acid (147 mg, 1.2 Eq, 1.08 mmol), Sodium carbonate (287 mg, 174 μL, 3 Eq, 2.70 mmol) and 1,1′-Bis(di-t-butylphosphino)ferrocene palladium dichloride (58.7 mg, 0.1 Eq, 90.1 μmol) in dioxane (12 mL) and H₂O (3 mL) was stirred at 50° C. for 2 hour under N₂. TLC (petroleum ether:ethyl acetate=5:1, UV) showed one main new spot was observed. The reaction was quenched with water (100 mL) and extracted with ethyl acetate (200 mL*2). The organic layer was washed with brine (50 mL) and dried over Na₂SO₄ and concentrated in vacuo to give 1-(4-(6-(benzyloxy)-4,4-difluoro-2-(p-tolyl)-3,4-dihydronaphthalen-1-yl)phenyl)-4-(dimethoxymethyl)piperidine as a brown oil. The brown oil was subjected to column chromatography over silica gel (gradient elution: 0-20% EtOAc). The desired fractions were collected, and concentrated to dryness in vacuo to give 1-(4-(6-(benzyloxy)-4,4-difluoro-2-(p-tolyl)-3,4-dihydronaphthalen-1-yl)phenyl)-4-(dimethoxymethyl)piperidine (570 mg, 918 μmol, 95.97% purity) as a white solid. LC-MS (ESI⁺) m/z: 596.3 (M+H)⁺.

Step 2: A mixture of 1-(4-(6-(benzyloxy)-4,4-difluoro-2-(p-tolyl)-3,4-dihydronaphthalen-1-yl)phenyl)-4-(dimethoxymethyl)piperidine (570 mg, 95.97% Wt, 1 Eq, 918 μmol), dihydrogen (1.85 mg, 1 Eq, 918 μmol), Pd/C (570 mg, 10% Wt, 0.583 Eq, 536 μmol) and Pd(OH)₂/C (570 mg, 20% Wt, 0.884 Eq, 812 μmol) in THF (10 mL) and MeOH (10 mL) was degassed and purged with H₂ for 3 times, and then the mixture was stirred at 25° C. for 4 hour under H₂ atmosphere (15 psi). The reaction was filtered and concentrated to dryness in vacuo to give 5-(4-(4-(dimethoxymethyl)piperidin-1-yl)phenyl)-8,8-difluoro-6-(p-tolyl)-5,6,7,8-tetrahydronaphthalen-2-ol (480 mg, 812 μmol, 88.4%, 85.89% purity) as a white solid. LC-MS (ESI⁺) m/z: 508.3 (M+H)⁺.

Step 3: The white solid was purified by SFC. Column: DAICEL CHIRALCEL OJ (250 mm*30 mm, 10 um); Condition: CO₂-EtOH (0.1% NH₃H₂O); At the beginning: B (30%); At the end: B (30%); Flow Rate (ml/min) δ0. The aqueous phase was lyophilized to dryness to give (5R,6S)-5-(4-(4-(dimethoxymethyl)piperidin-1-yl)phenyl)-8,8-difluoro-6-(p-tolyl)-5,6,7,8-tetrahydronaphthalen-2-ol (190 mg, 374 μmol, 46.1%, 100% purity) as a colorless oil and (5S,6R)-5-(4-(4-(dimethoxymethyl)piperidin-1-yl)phenyl)-8,8-difluoro-6-(p-tolyl)-5,6,7,8-tetrahydronaphthalen-2-ol (200 mg, 382 μmol, 47.1%, 97.01% purity) as a colorless oil. LC-MS (ESI⁺) m/z: 508.2 (M+H)⁺.

Step 4: To a solution of (5R,6S)-5-(4-(4-(dimethoxymethyl)piperidin-1-yl)phenyl)-8,8-difluoro-6-(p-tolyl)-5,6,7,8-tetrahydronaphthalen-2-ol (190 mg, 1 Eq, 374 μmol) in THF (10 mL) was added sulfuric acid (367 mg, 10 mL, 10% Wt, 1 Eq, 374 μmol). The mixture was stirred at 70° C. for 40 min. The mixture was adjusted to pH=8-9 by progressively adding NaHCO₃. The reaction mixture was quenched by addition 100 mL EtOAc at 20° C., and then diluted with 50 mL H₂O and extracted with EtOAc 50 mL (25 mL*2). The combined organic layers were washed with 10 mL (10 mL*1), dried over Na₂SO₄. The reaction was filtered and concentrated under reduced pressure to give 1-(4-((1R,2S)-4,4-difluoro-6-hydroxy-2-(p-tolyl)-1,2,3,4-tetrahydronaphthalen-1-yl)phenyl)piperidine-4-carbaldehyde (200 mg, 418 μmol, 96.5% purity) as a yellow oil. LC-MS (ESI+) m/z: 480.2 (M+H)⁺.

Step 5: A mixture of 1-(4-((1R,2S)-4,4-difluoro-6-hydroxy-2-(p-tolyl)-1,2,3,4-tetrahydronaphthalen-1-yl)phenyl)piperidine-4-carbaldehyde (200 mg, 1 Eq, 418 μmol), (S)-3-(1-oxo-5-(piperazin-1-yl)isoindolin-2-yl)piperidine-2,6-dione, PhSO₃H salt (137 mg, 1 Eq, 418 (mol) and sodium acetate (172 mg, 5 Eq, 2.09 mmol) in DCM (8 mL) and MeOH (8 mL) at 20° C. for 0.5 hour, pH=8, then acetic acid (75.3 mg, 71.7 μL, 3 Eq, 1.25 mmol) at 20° C. for 1 hour, pH=6, then added Sodium triacetoxyborohydride (177 mg, 124 μL, 2 Eq, 836 μmol) the mixture, and then the mixture was stirred at 25° C. for 16 hour. The reaction was quenched with water (50 mL) and extracted with ethyl acetate (50 mL*2). The reaction was concentrated to give (S)-3-(5-(4-((1-(4-((1R,2S)-4,4-difluoro-6-hydroxy-2-(p-tolyl)-1,2,3,4-tetrahydronaphthalen-1-yl)phenyl)piperidin-4-yl)methyl)piperazin-1-yl)-1-oxoisoindolin-2-yl)piperidine-2,6-dione as a yellow oil. The yellow oil was purified by preparative high-performance liquid chromatography. Condition: water(FA)-ACN. Column: Welch Xtimate C18 150*30 mm*5 um.

Begin B: 18%, End B: 48%. Gradient Time (min) 7; 100% B hold Time (min) 3.2, Flow Rate (ml/min) 25. The pure fractions were collected and the solvent was evaporated under vacuum. The aqueous layer was lyophilized to dryness to give (S)-3-(5-(4-((1-(4-((1*R, 2*S)-4,4-difluoro-6-hydroxy-2-(p-tolyl)-1,2,3,4-tetrahydronaphthalen-1-yl)phenyl)piperidin-4-yl)methyl)piperazin-1-yl)-1-oxoisoindolin-2-yl)piperidine-2,6-dione (106.9 mg, 135.6 μmol, 32.4%, 98.18% purity) as a white solid.

LCMS: calc. for C₄₆H₄₉F₂N₅O₄: 773.38, found: [M+H]⁺ 774.3.

HPLC: 98.18% purity at 220 nm.

¹H NMR (400 MHZ, DMSO-d₆) δ=10.97-10.94 (m, 1H), 9.75-9.71 (m, 1H), 8.13 (s, 0.516H), 7.53-7.49 (m, 1H), 7.06 (br s, 3H), 7.00 (d, J=7.9 Hz, 2H), 6.86 (s, 2H), 6.79-6.75 (m, 2H), 6.61-6.55 (m, 2H), 6.17-6.13 (m, 2H), 5.08-5.01 (m, 1H), 4.35-4.29 (m, 1H), 4.27 (br d, J=2.7 Hz, 1H), 4.22 (s, 1H), 3.62-3.50 (m, 3H), 3.27 (br s, 5H), 2.95-2.84 (m, 1H), 2.71-2.52 (m, 4H), 2.48-2.45 (m, 2H), 2.43-2.29 (m, 2H), 2.25 (s, 4H), 2.19 (br d, J=6.3 Hz, 2H), 1.99-1.91 (m, 1H), 1.79-1.71 (m, 2H), 1.68-1.59 (m, 1H), 1.20-1.09 (m, 2H)

### Example 31. Preparation of (I-321) (R)-3-(5-(4-((1-(4-((1S,2R)-4,4-difluoro-6-hydroxy-2-(p-tolyl)-1,2,3,4-tetrahydronaphthalen-1-yl)phenyl)piperidin-4-yl)methyl)piperazin-1-yl)-1-oxoisoindolin-2-yl)piperidine-2,6-dione

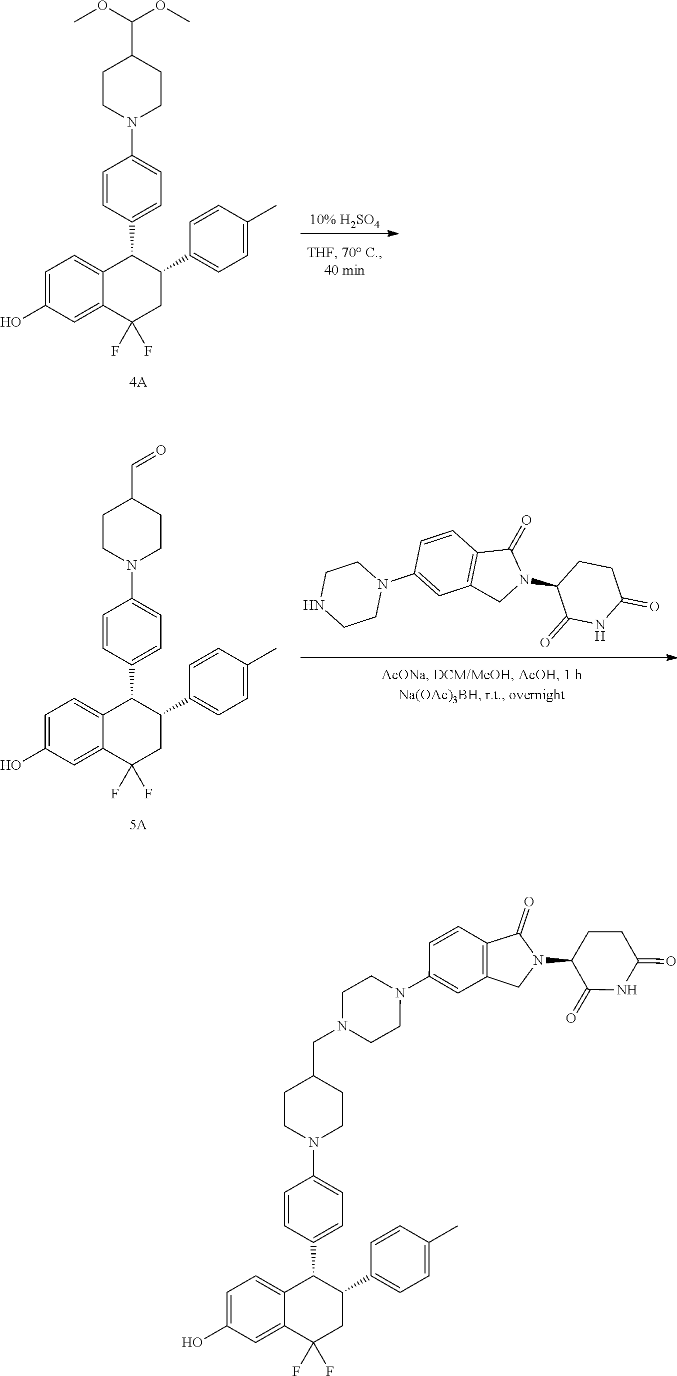 [SMILES: COC(OC)C1CCN(c2ccc([C@H]3c4ccc(O)cc4C(F)(F)C[C@H]3c3ccc(C)cc3)cc2)CC1 ; Cc1ccc([C@@H]2CC(F)(F)c3cc(O)ccc3[C@@H]2c2ccc(N3CCC(C=O)CC3)cc2)cc1 ; O=C1CC[C@H](N2Cc3cc(N4CCNCC4)ccc3C2=O)C(=O)N1 ; Cc1ccc([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)cc1]

Step 1: To a solution of (5S,6R)-5-(4-(4-(dimethoxymethyl)piperidin-1-yl)phenyl)-8,8-difluoro-6-(p-tolyl)-5,6,7,8-tetrahydronaphthalen-2-ol (200 mg, 1 Eq, 776 μmol) in THF (10 mL) was added sulfuric acid (761 mg, 10 mL, 10% Wt, 1 Eq, 776 μmol). The mixture was stirred at 70° C. for 40 min. The mixture was adjusted to pH=8-9 by progressively adding NaHCO₃. The reaction mixture was quenched by addition 100 mL EtOAc at 20° C., and then diluted with 50 mL H₂O and extracted with EtOAc 50 mL (25 mL*2). The combined organic layers were washed with 10 mL (10 mL*1), dried over Na₂SO₄. The reaction was filtered and concentrated under reduced pressure to give 1-(4-((1S,2R)-4,4-difluoro-6-hydroxy-2-(p-tolyl)-1,2,3,4-tetrahydronaphthalen-1-yl)phenyl)piperidine-4-carbaldehyde (195 mg, 400 μmol, 51.5%, 94.6% Purity) as a yellow oil. LC-MS (ESI+) m/z: 480.1 (M+H)⁺.

Step 2: A mixture of 1-(4-((1S,2R)-4,4-difluoro-6-hydroxy-2-(p-tolyl)-1,2,3,4-tetrahydronaphthalen-1-yl)phenyl)piperidine-4-carbaldehyde (195 mg, 1 Eq, 400 μmol), (S)-3-(1-oxo-5-(piperazin-1-yl)isoindolin-2-yl)piperidine-2,6-dione, PhSO₃H salt (131 mg, 1 Eq, 400 μmol) and sodium acetate (164 mg, 5 Eq. 2.00 mmol) in DCM (8 mL) and MeOH (8 mL) at 20° C. for 0.5 hour, pH=8, then acetic acid (72.0 mg, 68.6 μL, 3 Eq, 1.20 mmol) at 20° C. for 1 hour, pH=6, then added Sodium triacetoxyborohydride (169 mg, 118 μL, 2 Eq, 799 μmol) the mixture, and then the mixture was stirred at 25° C. for 16 hour. The reaction was quenched with water (50 mL) and extracted with ethyl acetate (50 mL*2). The reaction was concentrated to give (S)-3-(5-(4-((1-(4-((1S,2R)-4,4-difluoro-6-hydroxy-2-(p-tolyl)-1,2,3,4-tetrahydronaphthalen-1-yl)phenyl)piperidin-4-yl)methyl)piperazin-1-yl)-1-oxoisoindolin-2-yl)piperidine-2,6-dione as a yellow oil. The yellow oil was purified by preparative high-performance liquid chromatography. Condition: water(FA)-CAN. Column: Welch Xtimate C18 150*30 mm*5 um. Begin B: 26%, End B: 56%), Gradient Time (min) 7; 100% B hold Time (min) 3.2, Flow Rate (ml/min) 25. The pure fractions were collected and the solvent was evaporated under vacuum. The aqueous layer was lyophilized to dryness to give (R)-3-(5-(4-((1-(4-((1S,2R)-4,4-difluoro-6-hydroxy-2-(p-tolyl)-1,2,3,4-tetrahydronaphthalen-1-yl)phenyl)piperidin-4-yl)methyl)piperazin-1-yl)-1-oxoisoindolin-2-yl)piperidine-2,6-dione (102.8 mg, 129.5 μmol, 32.4%, 97.50% Purity) as a white solid.

LCMS: calc. for C₄₆H₄₉F₂N₅O₄: 773.38, found: [M+H]⁺ 774.3.

HPLC: 97.50% purity at 220 nm.

¹H NMR (400 MHZ, DMSO-d₆) δ=10.95 (s, 1H), 9.75-9.70 (m, 1H), 8.14 (s, 0.227H), 7.54-7.48 (m, 1H), 7.08-7.03 (m, 3H), 7.00 (d, J=8.1 Hz, 2H), 6.89-6.82 (m, 2H), 6.80-6.75 (m, 2H), 6.58 (d, J=8.8 Hz, 2H), 6.18-6.11 (m, 2H), 5.09-5.00 (m, 1H), 4.36-4.25 (m, 2H), 4.21 (s, 1H), 3.63-3.50 (m, 3H), 3.27 (br s, 5H), 2.95-2.84 (m, 1H), 2.68-2.53 (m, 3H), 2.48-2.44 (m, 3H), 2.43-2.29 (m, 2H), 2.25 (s, 4H), 2.21-2.16 (m, 2H), 1.99-1.91 (m, 1H), 1.78-1.71 (m, 2H), 1.68-1.60 (m, 1H), 1.21-1.09 (m, 2H)

### Example 32. Preparation of (1-328) (S)-3-(5-(4-((1-(4-((1S,2R)-4,4-difluoro-6-hydroxy-2-phenyl-1,2,3,4-tetrahydronaphthalen-1-yl)-2-fluorophenyl)piperidin-4-yl)methyl)piperazin-1-yl)-1-oxoisoindolin-2-yl)piperidine-2,6-dione and (I-194) (S)-3-(5-(4-((1-(4-((1R,2S)-4,4-difluoro-6-hydroxy-2-phenyl-1,2,3,4-tetrahydronaphthalen-1-yl)-2-fluorophenyl)piperidin-4-yl)methyl)piperazin-1-yl)-1-oxoisoindolin-2-yl)piperidine-2,6-dione

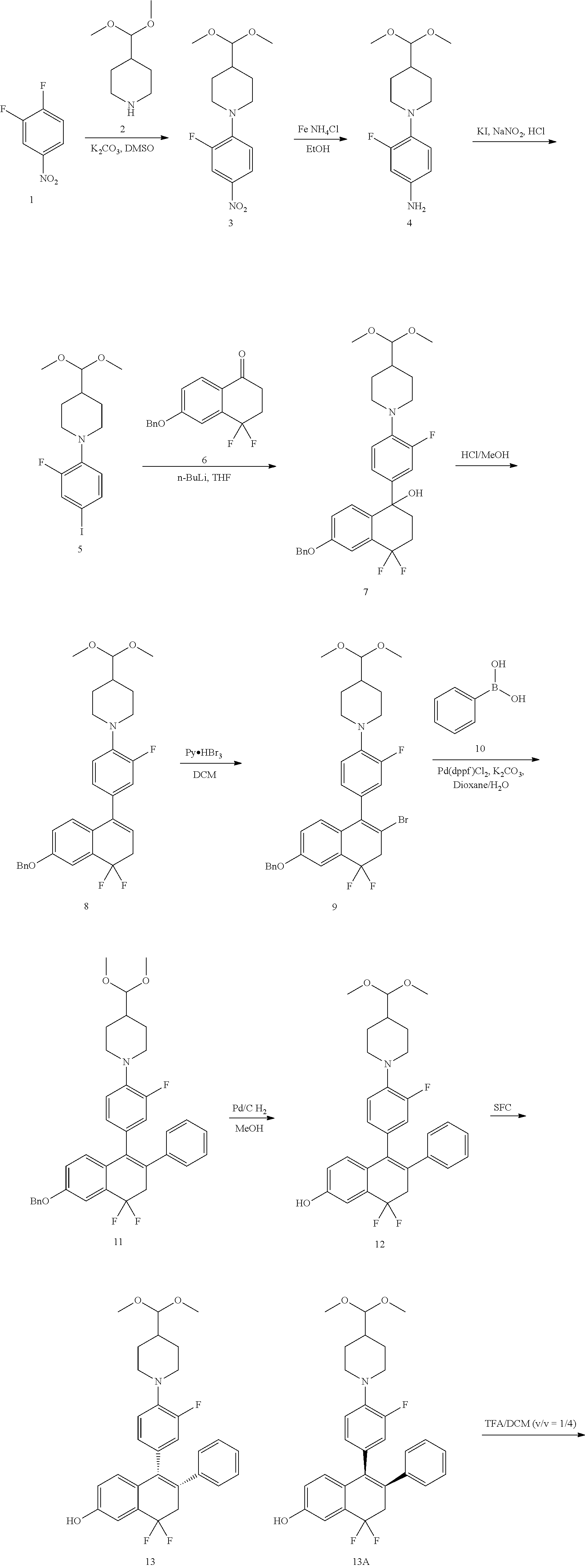

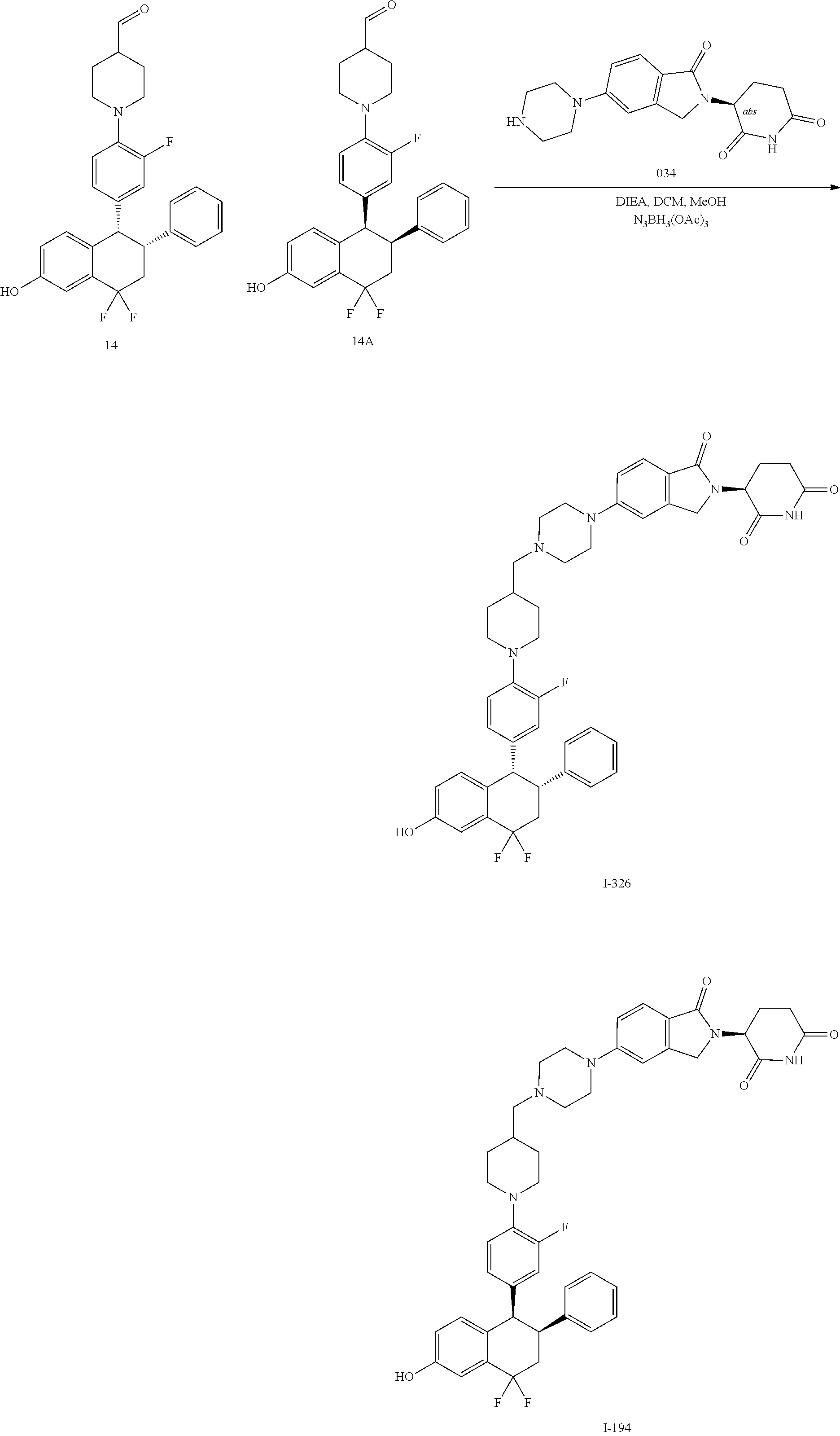 [SMILES: O=CC1CCN(c2ccc([C@H]3c4ccc(O)cc4C(F)(F)C[C@H]3c3ccccc3)cc2F)CC1 ; O=CC1CCN(c2ccc([C@@H]3c4ccc(O)cc4C(F)(F)C[C@@H]3c3ccccc3)cc2F)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccccc7)cc6F)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccccc7)cc6F)CC5)CC4)ccc3C2=O)C(=O)N1]

Step 1: To a solution of compound 1 (3.0 g, 18.86 mmol, 1 eq) and 2 (3.60 g, 22.63 mmol, 1.2 eq) in EtOAc (30 mL) was added Et₃N (5.72 g, 56.57 mmol, 3 eq). The mixture was stirred at 25° C. for 3 hr. The mixture was concentrated in reduced pressure. The residue was poured into water (30 mL). The aqueous phase was extracted with ethyl acetate (60 mL×3). The combined organic phase was washed with brine (30 mL×3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford compound 3 (4.43 g, 14.85 mmol, 78.75% yield) as yellow oil.

LC-MS (ESI+) m/z: 299.2 (M+H)⁺.

Step 2: To a solution of compound 3 (4.43 g, 14.85 mmol, 1.0 eq.) in EtOH (50 mL) and H₂O (17 mL) was added Fe (3.32 g, 59.35 mmol, 4 eq) and NH₄Cl (1.59 g, 29.67 mmol, 2 eq). The mixture was stirred at 80° C. for 5 hr. LCMS showed that the starting material was consumed and the desired product was detected. The residue was poured into water (55 mL). The aqueous phase was extracted with ethyl acetate (100 mL×3). The combined organic phase was washed with brine (55 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford compound 4 (3.72 g, 13.86 mmol, 93.44% yield) as yellow oil. The reaction mixture was filtered under reduced pressure to give a compound. Compound 4 (3.72 g, 13.86 mmol, 93.44% yield) was obtained as yellow oil.

LC-MS (ESI+) m/z: 269.2 (M+H)⁺.

Step 3: To a solution of compound 4 (3.72 g, 13.86 mmol, 1 eq) in MeCN (70 mL) and H₂O (70 mL) was added KI (6.90 g, 41.59 mmol, 3 eq), HCl (2 M, 6.93 mL, 1 eq) and NaNO₂ (2.87 g, 41.59 mmol, 3 eq). The mixture was stirred at 25° C. for 5 hr. LC-MS showed that the starting material was consumed and the desired product was detected. The residue was poured into water (70 mL). The aqueous phase was extracted with ethyl acetate (140 mL×3). The combined organic phase was washed with brine (70 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 5 (4.42 g, 11.66 mmol, 84.07% yield) as yellow oil. The reaction mixture was filtered under reduced pressure to give a compound. Compound 5 (4.42 g, 11.66 mmol, 84.07% yield) was obtained as yellow oil.

LC-MS (ESI+) m/z: 380.1 (M+H)⁺.

Step 4: To a solution of 5 (3.0 g, 7.91 mmol, 1 eq) in THF (35 mL) was added dropwise n-BuLi (2.5 M, 3.80 mL, 1.2 eq) at −78 C. After addition, the mixture was stirred at this temperature for 1 hr, and then compound 6 (2.28 g, 7.91 mmol, 1 eq) in THF (35 mL) was added dropwise at −78° C. The resulting mixture was stirred at −78° C. for 3 hr. LCMS showed that the starting material was consumed and the desired product was detected. The residue was poured into water (35 mL). The aqueous phase was extracted with ethyl acetate (70 mL×3). The combined organic phase was washed with brine (30 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 7 (2.12 g, 3.91 mmol, 49.48% yield) as yellow oil. The reaction mixture was filtered under reduced pressure to give a compound. Compound 7 (2.12 g, 3.91 mmol, 49.48% yield) was obtained as yellow oil.

LC-MS (ESI+) m/z: 542.3 (M+H)⁺.

Step 5: To a solution of compound 7 (2.12 g, 3.91 mmol, 1 eq) in HCl/MeOH (25 mL). The mixture was stirred at 25° C. for 3 hr. LCMS showed that the starting material was consumed and the desired product was detected. The reaction mixture was quenched by addition of water (8 mL), adjust pH to 7 by NaHCO₃, extracted with EtOAc (50 mL×3). The combined organic layers were washed with brine (8 mL), dried over Na₂SO₄, filtered and concentrated under reduced pressure to yield 8 (1.22 g, 2.33 mmol, 59.53% yield) which was used in the next step without further purification.

LC-MS (ESI+) m/z: 524.4 (M+H)⁺.

Step 6: To a solution of compound 8 (1.22 g, 2.33 mmol, 1 eq) in DCM (25 mL) was added Py·HBr₃ (745.21 mg, 2.33 mmol, 1 eq). The mixture was stirred at 25° C. for 1 hr. LCMS showed that the starting material was consumed and the desired product was detected. The residue was poured into water (25 mL). The aqueous phase was extracted with ethyl acetate (50 mL×3). The combined organic phase was washed with brine (25 mL), dried with anhydrous

Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 9 (888 mg, 1.47 mmol, 63.26% yield) as yellow oil. The reaction mixture was filtered under reduced pressure to give a compound. Compound 9 (888 mg, 1.47 mmol, 63.26% yield) was obtained as yellow oil.

LC-MS (ESI+) m/z: 604.2 (M+H)⁺.

Step 7: A mixture of compound 9 (888 mg, 1.47 mmol, 1 eq), compound 10 (179.71 mg, 1.47 mmol, leg), K₂CO₃ (407.40 mg, 2.95 mmol, 2 eq) and Pd(dppf)Cl₂ (107.85 mg, 147.39 μmol, 0.1 eq) in dioxane (10 mL) and H₂O (3 mL) was degassed and purged with N₂ for 3 times, and then the mixture was stirred at 80° C. for 3 hr under N₂ atmosphere. LCMS showed that the starting material was consumed and the desired product was detected. The reaction mixture was filtered under reduced pressure to give a residue. The residue was poured into H₂O (13 mL). The aqueous phase was extracted with ethyl acetate (26 mL×3). The combined organic phase was washed with brine (13 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 10 (800 mg, 1.33 mmol, 90.51% yield) as yellow oil.

LC-MS (ESI+) m/z: 600.4 (M+H)⁺.

Step 8: To a solution of compound 11 (591 mg, 985.52 μmol, 1 eq) in MeOH (10 mL) was added Pd/C (524.40 mg, 492.76 μmol, 10% purity, 0.5 eq). The mixture was stirred under H₂ at 25° C. and 15 Psi for 16 hr. LCMS showed that the starting material was consumed and the desired product was detected. The reaction mixture was filtered under reduced pressure to give residue. The residue was purified by prep-HPLC (column: Boston Green ODS 150*30 mm*5 um; mobile phase: [water (TFA)-ACN]; B %: 50%-80%, 12 min) to give compound 12 (200 mg, 390.95 μmol, 39.67% yield) was obtained as a white solid.

LC-MS (ESI+) m/z: 512.3 (M+H)⁺.

Step 9: Compound 13 and 13A (200 mg) was separated by chiral SFC(Column DAICEL CHIRALPAK AD (250 mm*30 mm, 10 um), Condition CO₂-EtOH (0.1% NH₃H₂O) Begin B 25% End B 25%) FlowRate (ml/min): 70) Compound 13 (70 mg, 136.83 μmol, 35.00% yield), compound 13A (120 mg, 234.57 μmol, 60.00% yield).

LC-MS (ESI+) m/z: 512.3 (M+H)⁺.

Step 10: Compound 14: To a solution of compound 13 (70 mg, 136.83 μmol, 1.0 eq) in DCM (2 mL) was added TFA (15.60 mg, 136.83 μmol, 10.16 μL, 1 eq). The mixture was stirred at 25° C. for 0.5 hr. LCMS showed that the starting material was consumed and the desired product was detected. The reaction liquid is dried with N₂ at 0° C. to give Compound 14 (42 mg, 90.22 μmol, 65.94% yield) was obtained as yellow oil which was used in the next step without further purification.

LC-MS (ESI+) m/z: 466.1 (M+H)⁺.

Step 11: Compound 14A: To a solution of compound 13A (70 mg, 136.83 μmol, 1 eq) in DCM (2 mL) was added TFA (15.60 mg, 136.83 μmol, 10.16 μL, 1 eq). The mixture was stirred at 25° C. for 0.5 hr. LCMS showed that the starting material was consumed and the desired product was detected. The reaction liquid is dried with N₂ at 0° C. to give Compound 14A (40 mg, 85.93 μmol, 62.80% yield) was obtained as yellow oil which was used in the next step without further purification.

LC-MS (ESI+) m/z: 466.1 (M+H)⁺.

Step 12: To a solution of compound 14 (40 mg, 85.93 μmol, 1 eq) in DCM (3 mL) and MeOH (3 mL) was added compound 034 (28.22 mg, 85.93 μmol, 1 eq) and DIEA (11.11 mg, 85.93 μmol, 14.97 μL, 1 eq) at 25° C. After addition, the mixture was stirred at this temperature for 0.5 hr, and then NaBH₃(OAc)₃ (36.42 mg, 171.86 μmol, 2 eq) was added at 25° C. The resulting mixture was stirred at 25° C. for 3 hr. The reaction mixture was concentrated to yield a residue. The residue was purified by preparative HPLC (column: Boston Green ODS 150*30 mm*5 um; mobile phase: [water(FA)-ACN]; B %: 18%-48%, 14 min), followed by lyophilization to yield I-328 (46.6 mg, 59.91 μmol, 69.72% yield) was obtained as a white solid. LC-MS (ESI+) m/z: 778.6 [M+H]⁺.

LCMS: calc. for C₄₅H₄₆F₃N₅O₄: 777.35, found: [M+H]⁺ 778.6.

HPLC: 97.12% purity at 220 nm.

¹H NMR (400 MHZ, METHANOL-d₄) δ ppm 8.36 (br s, 1H) 7.62 (d, J=8.46 Hz, 1H) 7.16-7.20 (m, 3H) 7.13 (s, 1H) 7.06-7.09 (m, 2H) 6.83-6.88 (m, 4H) 6.68 (t. J=8.76 Hz, 1H) 6.05-6.10 (m, 1H) 5.91-5.97 (m, 1H) 5.08 (dd, J=13.35, 5.13 Hz, 1H) 4.35-4.44 (m, 2H) 4.31 (br d, J=3.81 Hz, 1H) 3.66-3.73 (m, 1H) 3.39 (br d, J=4.53 Hz, 4H) 2.82-2.90 (m, 1H) 2.69-2.82 (m, 6H) 2.50-2.68 (m, 4H) 2.41-2.49 (m, 3H) 2.26-2.34 (m, 1H) 2.09-2.16 (m, 1H) 1.74-1.87 (m, 3H) 1.32-1.41 (m, 2H)

Step 13: To a solution of compound 14A (40 mg, 85.93 μmol, 1 eq) in DCM (3 mL) and MeOH (3 mL) was added compound 034 (28.22 mg, 85.93 μmol, 1 eq) and DIEA (11.11 mg, 85.93 μmol, 14.97 μL, 1 eq) at 25° C. After addition, the mixture was stirred at this temperature for 0.5 hr, and then NaBH₃(OAc)₃ (36.42 mg, 171.86 μmol, 2 eq) was added at 25° C. The resulting mixture was stirred at 25° C. for 3 hr. The reaction mixture was concentrated to yield a residue. The residue was purified by preparative HPLC (column: Boston Green ODS 150*30 mm*5 um; mobile phase: [water(FA)-ACN]; B %: 18%-48%, 14 min), followed by lyophilization to yield I-194 (42 mg, 53.99 μmol, 62.84% yield) was obtained as a white solid. LC-MS (ESI+) m/z: 778.5 [M+H]⁺.

LCMS: calc. for C₄₅H₄₆F₃N₅O₄: 777.35, found: [M+H]⁺ 778.5.

HPLC: 92.14% purity at 220 nm

¹H NMR (400 MHZ, METHANOL-d₄) δ ppm 8.39 (s, 1H) 7.62 (d, J=8.46 Hz, 1H) 7.17-7.22 (m, 3H) 7.13 (s, 1H) 7.06-7.10 (m, 2H) 6.82-6.88 (m, 4H) 6.65-6.73 (m, 1H) 6.06-6.11 (m, 1H) 5.95 (br d, J=13.83 Hz, 1H) 5.08 (br d, J=8.46 Hz, 1H) 4.36-4.44 (m, 2H) 4.31-4.34 (m, 1H) 3.68-3.73 (m, 1H) 3.38 (br s, 4H) 2.84-2.90 (m, 1H) 2.68-2.80 (m, 6H) 2.51-2.66 (m, 4H) 2.38-2.46 (m, 3H) 2.27-2.34 (m, 1H) 2.09-2.15 (m, 1H) 1.72-1.87 (m, 3H) 1.31-1.40 (m, 2H).

### Example 33. Preparation of (I-313) (S)-3-(5-(4-((1-(4-((1S,2R)-4,4-difluoro-2-(2-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

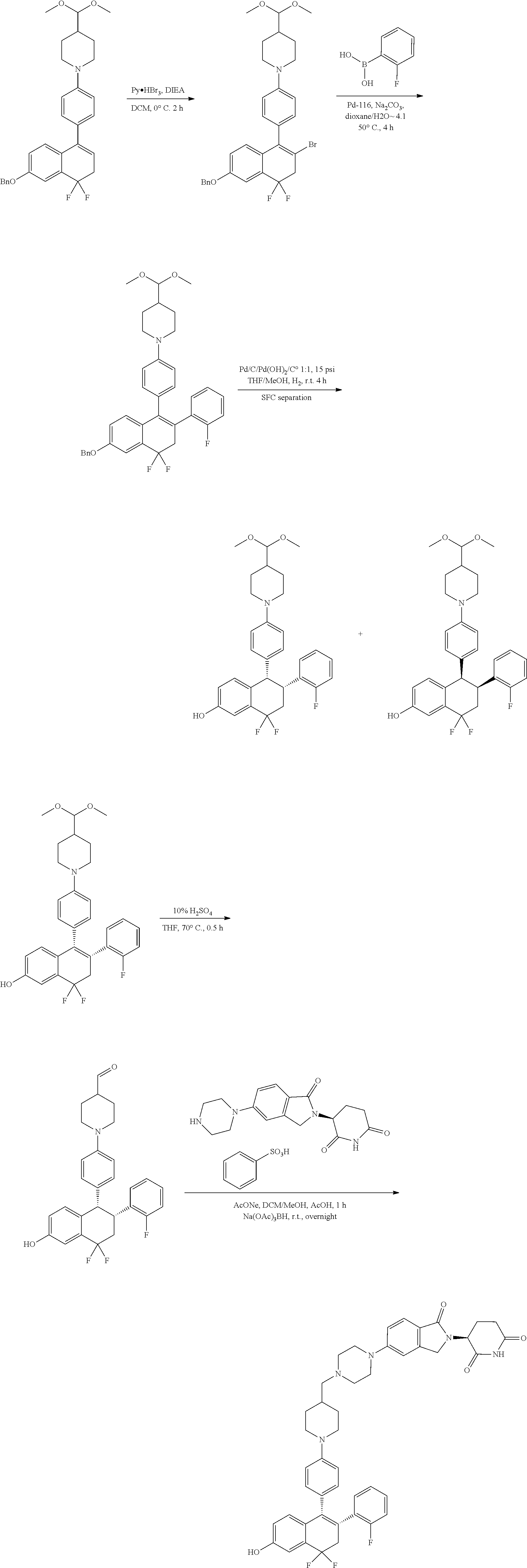

Step 1: To a mixture of 1-(4-(6-(benzyloxy)-4,4-difluoro-3,4-dihydronaphthalen-1-yl)phenyl)-4-(dimethoxymethyl)piperidine (7.8 g, 1 Eq, 15 mmol), DIEA (3.0 g, 4.0 mL, 1.5 Eq, 23 mmol) in DCM (25 mL) at 0° C., then Pyridinium bromide perbromide (8 g, 2 Eq. 0.03 mol) was added to the mixture at 0° C., the mixture was stirred at 0° C. for 2 hour. The mixture was treated with H₂O (500 mL), extracted with EtOAc (600 mL). The combined extracts was dried over anhydrous Na₂SO₄, filtered and concentrated to dryness in vacuo to give a residue. The residue was subjected to column chromatography over silica gel (gradient elution: 0˜30% ethyl acetate in petroleum ether). The pure fractions were collected and concentrated to dryness in vacuo to give 1-(4-(6-(benzyloxy)-2-bromo-4,4-difluoro-3,4-dihydronaphthalen-1-yl)phenyl)-4-(dimethoxymethyl)piperidine (8 g, 0.01 mol, 80%, 87.8% purity) as a white solid

LC-MS (ESI⁺) m/z: 586.2 (M+H)⁺.

Step 2:1,1′-Bis(di-t-butylphosphino)ferrocene palladium dichloride (0.1 g, 0.1 Eq, 0.2 mmol) was added to the mixture of 1-(4-(6-(benzyloxy)-2-bromo-4,4-difluoro-3,4-dihydronaphthalen-1-yl)phenyl)-4-(dimethoxymethyl)piperidine (1 g, 1 Eq, 2 mmol), (2-Fluorophenyl)boranediol (0.2 g, 1 Eq, 2 mmol) and Na₂CO₃ (0.5 g, 3 Eq, 5 mmol) in dioxane (8 mL) and H₂O (2 mL) at r.t. N₂ was bubbled into the mixture for 5 min. The reaction mixture was heated at 50° C. for 4 hour. Then the reaction was cooled to room temperature. The mixture was treated with H₂O (100 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜30% ethyl acetate in petroleum ether). The pure fractions were collected and concentrated to dryness in vacuo to give 1-(4-(6-(benzyloxy)-4,4-difluoro-2-(2-fluorophenyl)-3,4-dihydronaphthalen-1-yl)phenyl)-4-(dimethoxymethyl)piperidine (900 mg, 1.43 mmol, 100%, 95.6% Purity) as a yellow oil.

LC-MS (ESI⁺) m/z: 600.3 (M+H)⁺.

Step 3: A mixture of 1-(4-(6-(benzyloxy)-4,4-difluoro-2-(2-fluorophenyl)-3,4-dihydronaphthalen-1-yl)phenyl)-4-(dimethoxymethyl)piperidine (700 mg, 1 Eq, 1.12 mmol), Pd/C (350 mg, 10% Wt, 0.295 Eq, 329 μmol), Pd(OH)₂/C (350 mg, 20% Wt, 0.447 Eq, 498 μmol), dihydrogen (2.25 mg, 1 Eq, 1.12 mmol) in THF (4 mL) and MeOH (4 mL). The suspension was degassed and purged with H₂ for 3 times. The mixture was stirred under H₂ (15 psi) at 25° C. for 4 hour. The reaction was filtered and concentrated to dryness in vacuo to give a white solid. The white solid was subjected by SFC: Column: DAICEL CHIRALCEL OJ (250 mm*30 mm, 10 um); Mobile phase: A: Supercritical CO₂, B: MeOH (0.1% NH₃H₂O), A:B=30:30 at 70 mL/min. The aqueous phase was lyophilized to dryness to give (5R,6S)-5-(4-(4-(dimethoxymethyl)piperidin-1-yl)phenyl)-8,8-difluoro-6-(2-fluorophenyl)-5,6,7,8-tetrahydronaphthalen-2-ol (120 mg, 231 μmol, 40.4%, 98.5% Purity) and (5S,6R)-5-(4-(4-(dimethoxymethyl)piperidin-1-yl)phenyl)-8,8-difluoro-6-(2-fluorophenyl)-5,6,7,8-tetrahydronaphthalen-2-ol (150 mg, 293 μmol, 51.2%, 100% Purity) as a yellow solid.

LC-MS (ESI⁺) m/z: 512.2 (M+H)⁺.

Step 4: A mixture of (5S,6R)-5-(4-(4-(dimethoxymethyl)piperidin-1-yl)phenyl)-8,8-difluoro-6-(2-fluorophenyl)-5,6,7,8-tetrahydronaphthalen-2-ol (100 mg, 1 Eq, 193 μmol) in 10% H₂SO₄ (1.5 mL) and THF (1.5 mL), then the mixture was stirred at 60° C. for 0.5 hour. The solvent was removed under vacuum. The aqueous solution was added NaHCO₃ to adjust pH=6. The residue was dissolved in water (20 mL) and extracted by ethyl acetate (20 mL). The combined organic layer was dried over Na₂SO₄, filtered and concentrated under vacuum to give 1-(4-((1S,2R)-4,4-difluoro-2-(2-fluorophenyl)-6-hydroxy-1,2,3,4-tetrahydronaphthalen-1-yl)phenyl)piperidine-4-carbaldehyde (80 mg, 0.17 mmol, 89%) as a yellow oil.

LC-MS (ESI+) m/z: 466.1 (M+H)+

Step 5: A mixture of 1-(4-((1S,2R)-4,4-difluoro-2-(2-fluorophenyl)-6-hydroxy-1,2,3,4-tetrahydronaphthalen-1-yl)phenyl)piperidine-4-carbaldehyde (80 mg, 1 Eq, 0.17 mmol), (S)-3-(1-oxo-5-(piperazin-1-yl)isoindolin-2-yl)piperidine-2,6-dione benzenesulfonate (0.11 g, 1.3 Eq, 0.22 mmol) and sodium acetate (70 mg, 5 Eq, 0.86 mmol) and Acetic acid (31 mg, 30 μL, 3 Eq, 0.52 mmol) in DCM (2 mL) and MeOH (2 mL) at 25° C. for 1 hour, then sodium triacetoxyborohydride (73 mg, 2 Eq, 0.34 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 19%-49%, 7 min). The aqueous phase was lyophilized to dryness to give (S)-3-(5-(4-((1-(4-((1S,2R)-4,4-difluoro-2-(2-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (99.3 mg, 127 μmol, 74%, 99.251% purity) as a white solid.

LC-MS (ESI+) m/z: 778.2 (M+H)+

HPLC: 99.251%, purity at 220 nm.

¹H NMR (400 MHZ, DMSO-d₆) δ=10.96 (s, 1H), 9.79 (br s, 1H), 8.14 (s, 0.373H), 7.52 (d, J=8.6 Hz, 1H), 7.32-7.16 (m, 2H), 7.12-7.02 (m, 3H), 6.98-6.84 (m, 3H), 6.65-6.50 (m, 3H), 6.18 (d, J=8.5 Hz, 2H), 5.05 (dd, J=5.1, 13.3 Hz, 1H), 4.38-4.28 (m, 2H), 4.26-4.16 (m, 1H), 3.86 (br d, J=14.7 Hz, 1H), 3.60-3.46 (m, 3H), 3.34-3.19 (m, 8H), 2.98-2.84 (m, 1H), 2.83-2.67 (m, 1H), 2.64-2.54 (m, 2H), 2.43-2.27 (m, 2H), 2.20 (br d, J=6.7 Hz, 2H), 2.02-1.90 (m, 1H), 1.75 (brd, J=12.3 Hz, 2H), 1.66 (br d, J=9.4 Hz, 1H), 1.25-1.07 (m, 2H).

### Example 34. Preparation of (1-312) (S)-3-(5-(4-((1-(4-((1R,2S)-4,4-difluoro-2-(2-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

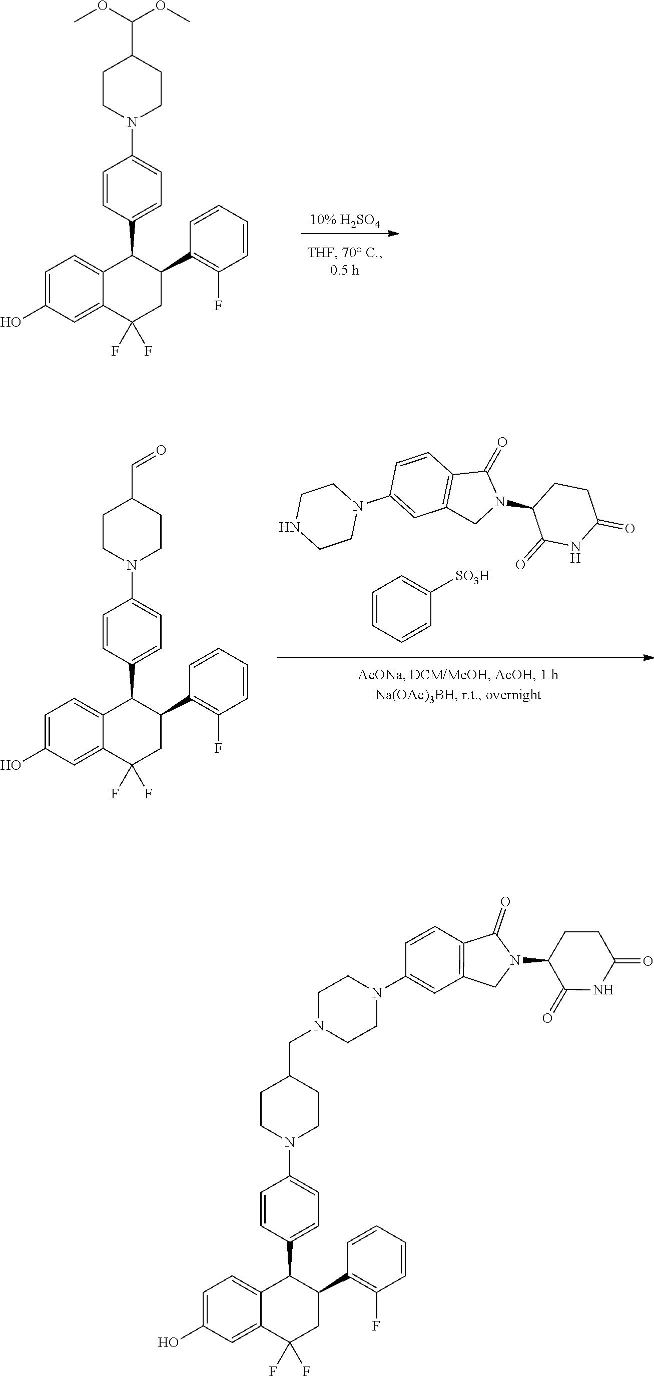 [SMILES: COC(OC)C1CCN(c2ccc([C@@H]3c4ccc(O)cc4C(F)(F)C[C@@H]3c3ccccc3F)cc2)CC1 ; O=CC1CCN(c2ccc([C@@H]3c4ccc(O)cc4C(F)(F)C[C@@H]3c3ccccc3F)cc2)CC1 ; O=C1CC[C@H](N2Cc3cc(N4CCNCC4)ccc3C2=O)C(=O)N1 ; O=S(=O)(O)c1ccccc1 ; O=C1CC[C@H](N2Cc3cc(N4CCN(CC5CCN(c6ccc([C@@H]7c8ccc(O)cc8C(F)(F)C[C@@H]7c7ccccc7F)cc6)CC5)CC4)ccc3C2=O)C(=O)N1]

Step 1: A mixture of (5R,6S)-5-(4-(4-(dimethoxymethyl)piperidin-1-yl)phenyl)-8,8-difluoro-6-(2-fluorophenyl)-5,6,7,8-tetrahydronaphthalen-2-ol (100 mg, 1 Eq, 195 μmol) in 10% H₂SO₄ (1.5 mL) and THF (1.5 mL), then the mixture was stirred at 60° C. for 0.5 hour. The solvent was removed under vacuum. The aqueous solution was added NaHCO₃ to adjust pH=6. The residue was dissolved in water (20 mL) and extracted by ethyl acetate (20 mL). The combined organic layer was dried over Na₂SO₄, filtered and concentrated under vacuum to give 1-(4-((1R,2S)-4,4-difluoro-2-(2-fluorophenyl)-6-hydroxy-1,2,3,4-tetrahydronaphthalen-1-yl)phenyl)piperidine-4-carbaldehyde (80 mg, 0.17 mmol, 88%) as a yellow oil.

LC-MS (ESI+) m/z: 466.1 (M+H)+

Step 2: A mixture of 1-(4-((1R,2S)-4,4-difluoro-2-(2-fluorophenyl)-6-hydroxy-1,2,3,4-tetrahydronaphthalen-1-yl)phenyl)piperidine-4-carbaldehyde (80 mg, 1 Eq, 0.17 mmol), (R)-3-(1-oxo-5-(piperazin-1-yl)isoindolin-2-yl)piperidine-2,6-dione benzenesulfonate (0.11 g, 1.3 Eq, 0.22 mmol) and sodium acetate (70 mg, 5 Eq, 0.86 mmol) and acetic acid (31 mg, 30 μL, 3 Eq, 0.52 mmol) in DCM (2 mL) and MeOH (2 mL) at 25° C. for 1 hour, then sodium triacetoxyborohydride (73 mg, 2 Eq, 0.34 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 19%-49%, 7 min). The aqueous phase was lyophilized to dryness to give (S)-3-(5-(4-((1-(4-((1R,2S)-4,4-difluoro-2-(2-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (51 mg, 65 μmol, 38%, 99.111% purity) as a white solid.

LC-MS (ESI+) m/z: 778.2 (M+H)+

HPLC: 99.111%, purity at 220 nm.

¹H NMR (400 MHZ, DMSO-d₆) δ=10.96 (s, 1H), 9.89-9.69 (m, 1H), 8.16 (s, 0.605H), 7.52 (d, J=8.6 Hz, 1H), 7.32-7.17 (m, 2H), 7.11-7.03 (m, 3H), 6.98-6.83 (m, 3H), 6.59 (d, J=8.8 Hz, 2H), 6.53 (t, J=7.3 Hz, 1H), 6.18 (d, J=8.7 Hz, 2H), 5.05 (dd, J=5.2, 13.4 Hz, 1H), 4.39-4.28 (m, 2H), 4.26-4.15 (m, 1H), 3.85 (br dd, J=3.0, 14.8 Hz, 1H), 3.60-3.48 (m, 3H), 3.30-3.16 (m, 8H), 2.96-2.84 (m, 1H), 2.79-2.67 (m, 1H), 2.65-2.53 (m, 2H), 2.40-2.29 (m, 2H), 2.19 (br d, J=6.9 Hz, 2H), 2.01-1.91 (m, 1H), 1.75 (br d, J=13.0 Hz, 2H), 1.69-1.58 (m, 1H), 1.23-1.08 (m, 2H).

### Example 35. Preparation of (I-323) (S)-3-(5-(4-((1-(4-((1S,2R)-4,4-difluoro-6-hydroxy-2-(o-tolyl)-1,2,3,4-tetrahydronaphthalen-1-yl)phenyl)piperidin-4-yl)methyl)piperazin-1-yl)-1-oxoisoindolin-2-yl)piperidine-2,6-dione and (I-322) (S)-3-(5-(4-((1-(4-((1R,2S)-4,4-difluoro-6-hydroxy-2-(o-tolyl)-1,2,3,4-tetrahydronaphthalen-1-yl)phenyl)piperidin-4-yl)methyl)piperazin-1-yl)-1-oxoisoindolin-2-yl)piperidine-2,6-dione

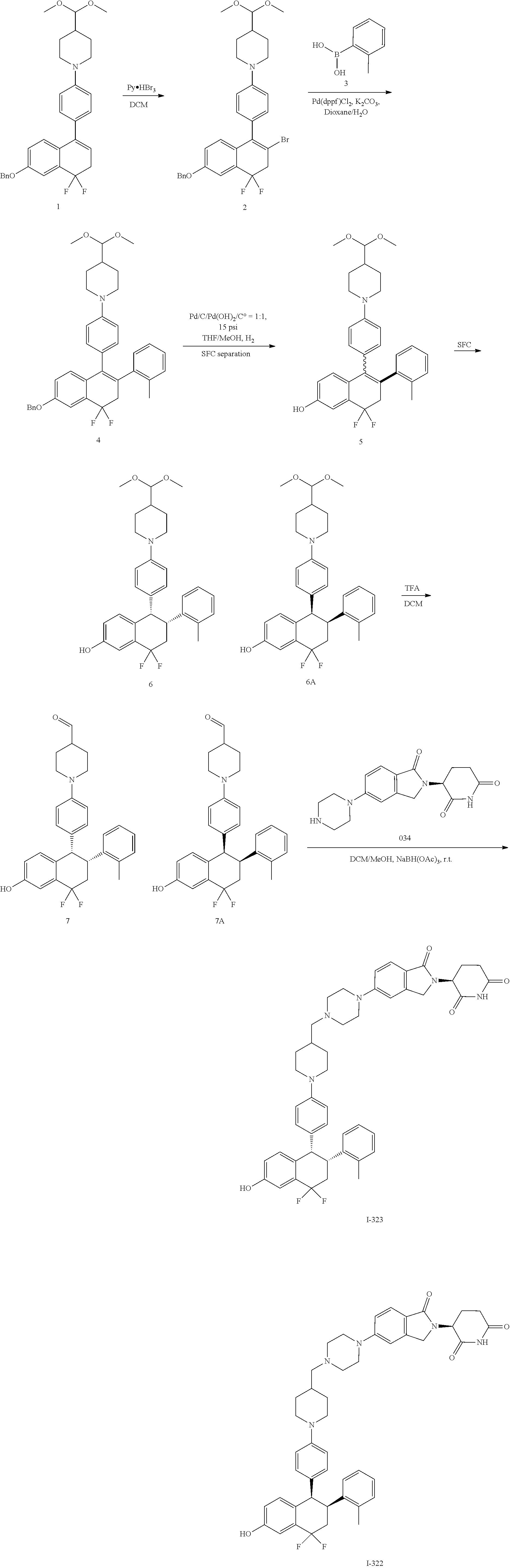

Step 1: To a solution of 1 (2.00 g, 3.96 mmol, 1.0 eq.) in DCM (20 mL) was added Pyridinium bromide perbromide (759 mg, 2.37 mmol, 0.6 eq.). The mixture was stirred at 20° C. for 1 hour. LCMS showed the reaction was completed. The reaction mixture was quenched by addition water 30 mL at 20° C., then diluted with water 30 mL and extracted with DCM 90 mL (30 mL×3). The combined organic layers were washed with DCM 30 mL (10 mL×3), dried over Na₂SO₄, filtered and concentrated under reduced pressure to give a residue. The crude material was purified on silica gel column chromatography (from pure PE to PE/EtOAc=6/1) to give compound 2 (1.50 g, 1.8 mmol, 45% yield, 70% Purity) as a yellow solid.

LC-MS (ESI⁺) m/z: 506.3 (M+H)⁺

Step 2: A mixture of 2 (400 mg, 684 μmol, 1.0 eq.), compound 3 (112 mg, 821 μmol, 1.2 eq.), PdCl₂(dppf) (50.1 mg, 68.4 μmol, 0.1 eq.) and K₂CO₃ (236 mg, 1.71 mmol, 2.5 eq.) in dioxane (16 mL) and water (4 mL) was degassed and purged with N₂ for 3 times, and then the mixture was stirred at 60° C. for 3 hour under N₂ atmosphere. LCMS showed the reaction was completed. The reaction mixture was quenched by addition EA 10 mL at 20° C., and then diluted with water 10 mL and extracted with EA 90 mL (30 mL×3). The combined organic layers were washed with EA 15 mL (5 mL×3), dried over Na₂SO₄, filtered and concentrated under reduced pressure to give a residue. The crude material was purified on silica gel column chromatography (from pure PE to PE/EtOAc=4/1) to give compound 4 (300 mg, 0.40 mmol, 59%, 80% Purity) as a yellow solid.

LC-MS (ESI⁺) m/z: 596.2 (M+H)⁺

Step 3: To a solution of compound 4 (300 mg, 504 μmol, 1.0 eq.) in THF (3 mL) and MeOH (3 mL) was added Pd/C (10 wt %, 0.05 g) and Pd(OH)₂/C (10 wt %, 0.07 g) under H₂ atmosphere. The suspension was degassed and purged with H₂ for 3 times. The mixture was stirred under H₂ (15 Psi.) at 25° C. for 4 hours. LCMS showed the reaction was completed. The crude was purified by prep.HPLC together (Welch Xtimate C18 150*25 mm*5 um, water(FA)-ACN as a mobile phase, from 33% to 63%, Gradient Time (min): 11, Flow Rate (ml/min): 25) to give compound 5 (60 mg, 0.12 mmol, 23%) as a white solid.

LC-MS (ESI⁺) m/z: 508.3 (M+H)⁺

Step 4: The compound 5 (60 mg, 1 eq., 0.12 mmol) was purified by SFC(Column: DAICEL CHIRALCEL OD-H (250 mm*30 mm, 10 um); Mobile phase: 0.1% NH₃H₂O ETOH; from 25% to 25%; Flow rate: 60 mL/min) to give compound 6 (25 mg, 49 μmol, 42% yield) and compound 6A (25 mg, 49 μmol, 42% yield) both as white solid.

compound 6: LC-MS (ESI+) m/z: 508.3 (M+H)⁺

compound 6A: LC-MS (ESI+) m/z: 508.3 (M+H)⁺

Step 5: Compound 7: To a solution of compound 6 (10 mg, 19.70 μmol, 1.0 eq) in DCM (3 mL) was added TFA 2.25 mg, 19.70 μmol, 1.0 eq). The mixture was stirred at 25° C. for 0.2 hr. LCMS showed that the starting material was consumed and the desired product was detected. The reaction liquid is dried with N₂ at 0° C. to give Compound 7 (8.2 mg, 17.77 μmol, 90.18% yield) was obtained as yellow oil which was used in the next step without further purification.

LC-MS (ESI+) m/z: 462.2 (M+H)⁺.

Step 6: Compound 7A: To a solution of compound 6A (10 mg, 19.70 μmol, 1.0 eq) in DCM (2 mL) was added TFA (2.25 mg, 19.70 μmol, 1.0 eq). The mixture was stirred at 25° C. for 0.2 hr. LCMS showed that the starting material was consumed and the desired product was detected. The reaction liquid is dried with N₂ at 0° C. to give compound 7A (8.5 mg, 18.42 μmol, 93.48% yield) was obtained as yellow oil which was used in the next step without further purification.

LC-MS (ESI+) m/z: 462.2 (M+H)⁺.

Step 7: To a solution of compound 7 (9.55 mg, 20.68 μmol, 1.0 eq) in DCM (3 mL) and MeOH (3 mL) was added compound 034 (6.79 mg, 20.68 μmol, 1.0 eq) and DIEA (2.67 mg, 20.68 μmol, 1.0 eq) at 25° C. After addition, the mixture was stirred at this temperature for 0.5 hr, and then NaBH(OAc) 3 (8.77 mg, 41.37 μmol, 2.0 eq) was added at 25° C. The resulting mixture was stirred at 25° C. for 1 hr. The reaction mixture was concentrated to yield a residue. The residue was purified by preparative HPLC (column: Boston Green ODS 150*30 mm*5 um; mobile phase: [water(FA)-ACN]; B %: 26%-36%, 12 min), followed by lyophilization to yield I-323 (6.7 mg, 8.66 μmol, 41.86% yield) was obtained as a white solid. LC-MS (ESI+) m/z: 774.6 [M+H]⁺.

LCMS: calc. for C₄₆H₄₉F₂N₅O₄: 773.38, found: [M+H]⁺ 774.6.

HPLC: 97.34% purity at 220 nm.

¹H NMR (400 MHZ, METHANOL-d₄) δ ppm 8.32-8.36 (m, 1H), 7.60-7.72 (m, 1H), 7.30-7.56 (m, 1H), 7.20-7.25 (m, 1H), 7.06-7.18 (m, 4H), 6.80-6.93 (m, 3H), 6.66 (d, J=8.63 Hz, 2H), 6.23 (d, J=8.50 Hz, 1H), 6.15 (d, J=7.63 Hz, 1H), 5.12 (dd, J=13.20, 5.19 Hz, 1H), 4.33-4.48 (m, 3H), 3.84-3.94 (m, 1H), 3.58 (br d, J=10.76 Hz, 2H), 3.43-3.51 (m, 4H), 2.86-2.99 (m, 5H), 2.70-2.84 (m, 2H), 2.57-2.69 (m, 4H), 2.40-2.53 (m, 4H), 2.13-2.27 (m, 2H), 1.79-1.94 (m, 3H), 1.33-1.42 (m, 2H).

Step 8: To a solution of compound 7A (9.91 mg, 21.48 μmol, 1.0 eq) in DCM (3 mL) and MeOH (3 mL) was added compound 034 (7.05 mg, 21.48 μmol, 1.0 eq) and DIEA (2.78 mg, 21.48 μmol, 1.0 eq) at 25° C. After addition, the mixture was stirred at this temperature for 0.5 hr, and then NaBH(OAc); (9.11 mg, 42.96 μmol, 2.0 eq) was added at 25° C. The resulting mixture was stirred at 25° C. for 1 hr. The reaction mixture was concentrated to yield a residue. The residue was purified by pre-HPLC (column: Boston Green ODS 150*30 mm*5 um; mobile phase: [water (FA)-ACN]; B %: 26%-36%, 11 min), followed by lyophilization to yield I-322 (11.1 mg, 14.34 μmol, 66.77% yield) was obtained as a white solid. LC-MS (ESI⁺) m/z: 774.6 [M+H]⁺.

LCMS: calc. for C₄₆H₄₉F₂N₅O₄: 773.38, found: [M+H]⁺ 774.6.

HPLC: 95.85% purity at 220 nm

¹H NMR (400 MHz, METHANOL-d₄) δ ppm 8.30 (br s, 1H), 7.65 (d, J=8.88 Hz, 1H), 7.39-7.44 (m, 1H), 7.20 (d, J=7.75 Hz, 1H), 7.04-7.14 (m, 4H), 6.78-6.89 (m, 3H), 6.64 (d, J=8.63 Hz, 2H), 6.19-6.21 (m, 1H), 6.12 (d, J=7.63 Hz, 1H), 5.09 (dd, J=13.26, 5.13 Hz, 1H), 4.30-4.41 (m, 3H), 3.84-3.90 (m, 1H), 3.52-3.57 (m, 2H), 3.44 (br s, 4H), 2.84-2.92 (m, 5H), 2.71-2.80 (m, 2H), 2.54-2.63 (m, 4H), 2.42-2.49 (m, 4H), 2.12-2.22 (m, 2H), 1.80-1.90 (m, 3H), 1.30-1.38 (m, 2H),

### Example 36. Preparation of (I-324) (S)-3-(5-(4-((1-(4-((1R,2S)-2-(2,5-difluoro-4-methylphenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

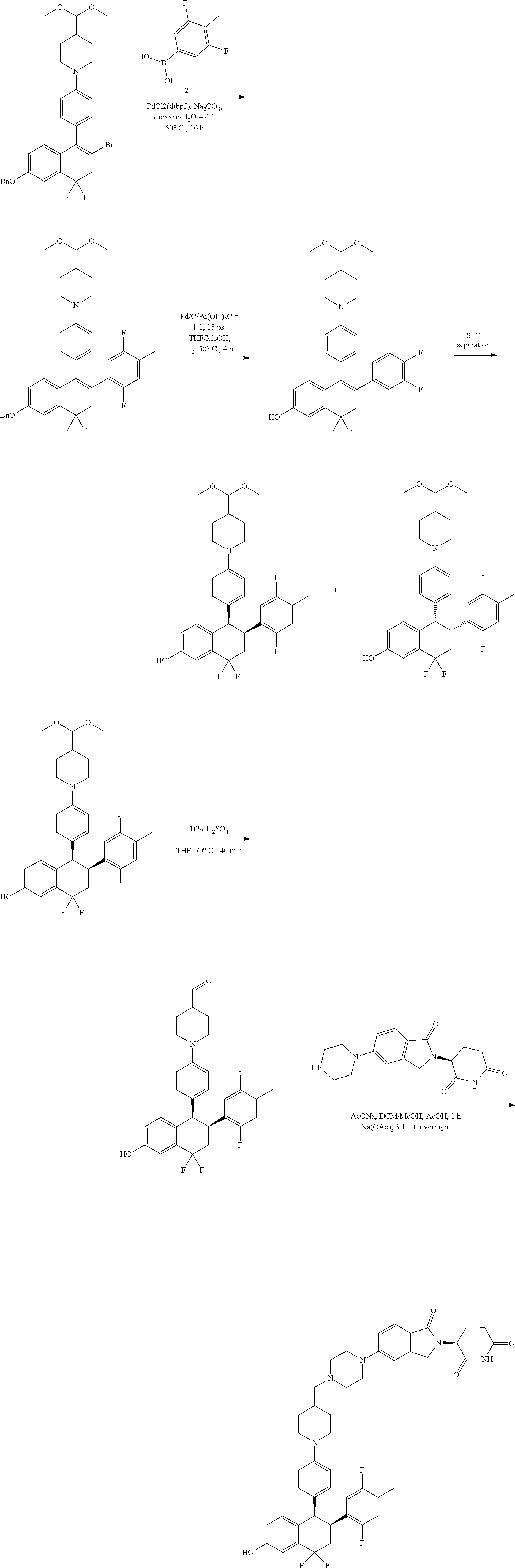

Step 1: A mixture of 1,1′-Bis(di-t-butylphosphino)ferrocene palladium dichloride (199 mg, 0.1 Eq, 305 μmol), (2,5-difluoro-4-methylphenyl) boronic acid (630 mg, 1.2 Eq, 3.6 mmol), sodium carbonate (971 mg, 3 Eq, 9.2 mmol) and 1-(4-(6-(benzyloxy)-2-bromo-4,4-difluoro-3,4-dihydronaphthalen-1-yl)phenyl)-4-(dimethoxymethyl)piperidine (1.8 g, 1.0 Eq, 3 mmol) in 1,4-dioxane (12 mL) and H₂O (3 mL) was degassed and purged with N₂ for 3 times, and then the mixture was stirred at 50° C. for 16 h under N₂ atmosphere. TLC (petroleum ether:ethyl acetate=3:1, R_{f}=0.6 UV) showed one main new spot was observed. The resulting solution was treated with 50 mL H₂O and was extracted with 100 mL ethyl acetate. The organic layers were dried with anhydrous Na₂SO₄, filtered, and concentrated to give the crude product. The residue was purified by column chromatography (SiO₂, Petroleum ether/Ethyl acetate=10/0 to 8/2). The reaction mixture was filtered and concentrated to dryness in vacuum to give 1-(4-(6-(benzyloxy)-2-(2,5-difluoro-4-methylphenyl)-4,4-difluoro-3,4-dihydronaphthalen-1-yl)phenyl)-4-(dimethoxymethyl)piperidine (1.88 g, 2.98 mmol, 98% yield) as a white solid.

Step 2: A mixture of 1-(4-(6-(benzyloxy)-2-(2,5-difluoro-4-methylphenyl)-4,4-difluoro-3,4-dihydronaphthalen-1-yl)phenyl)-4-(dimethoxymethyl)piperidine (1.8 g, 1 Eq, 2.8 mmol), palladium on carbon (900 mg, 10% Wt, 0.3 Eq, 846 μmol), and Pd(OH)₂/C (900 mg, 20% Wt, 0.45 Eq, 1.28 mmol) in MeOH (20 mL) and THE (20 mL) was degassed under N₂ atmosphere, The suspension was degassed and purged with H₂ for 3 times. The mixture was stirred under H₂ (15 Psi) at 50° C. for 4 h. The reaction mixture was filtered and concentrated under reduced pressure to give a residue. The residue was concentrated under reduced pressure to give (5R,6S)-6-(2,5-difluoro-4-methylphenyl)-5-(4-(4-(dimethoxymethyl)piperidin-1-yl)phenyl)-8,8-difluoro-5,6,7,8-tetrahydronaphthalen-2-ol as a yellow oil, which was further separated by SFC (condition: column: DAICEL CHIRALPAK IG (250 mm*30 mm, 10 um); mobile phase: [CO2-i-PrOH (0.1% NH₃H₂O)]; B %: 30%-30%) to give (5R,6S)-6-(2,5-difluoro-4-methylphenyl)-5-(4-(4-(dimethoxymethyl)piperidin-1-yl)phenyl)-8,8-difluoro-5,6,7,8-tetrahydronaphthalen-2-ol (300 mg, 531 μmol, 19%, 96% Purity) and (5S,6R)-6-(2,5-difluoro-4-methylphenyl)-5-(4-(4-(dimethoxymethyl)piperidin-1-yl)phenyl)-8,8-difluoro-5,6,7,8-tetrahydronaphthalen-2-ol (250 mg, 444 μmol, 16%, 97% Purity) as yellow solid. LC-MS (ESI+) m/z: 544.2 (M+H)⁺

Step 3: A mixture of (1S,2R)-2-(2,5-difluoro-4-methyl-phenyl)-1-[4-[4-(dimethoxymethyl)-1-piperidyl]phenyl]-4,4-difluoro-tetralin-6-ol (100 mg, 184 μmol, 1 eq) was added in THF (3 mL) and 10% H₂SO₄ (3 mL) and then the mixture was stirred at 70° C. for 40 min. The resulting solution was quenched with 10 mL NaHCO₃, the mixture was treated with 50 mL H₂O and was extracted with ethyl acetate 100 mL. The organic layers were dried with anhydrous Na₂SO₄, filtered, and concentrated to give 1-[4-[(1S,2R)-2-(2,5-difluoro-4-methyl-phenyl)-4,4-difluoro-6-hydroxy-tetralin-1-yl]phenyl]piperidine-4-carbaldehyde (90 mg, 181 μmol, 98% yield) was obtained as a white solid. LC-MS (ESI+) m/z: 498.1 (M+H)+.

Step 4: A mixture of (S)-3-(1-oxo-5-(piperazin-1-yl)isoindolin-2-yl)piperidine-2,6-dione (136 mg, 271.34 μmol, 1.5 eq, PhSO₃H salt), 1-[4-[(1*S, 2*R)-2-(2,5-difluoro-4-methyl-phenyl)-4,4-difluoro-6-hydroxy-tetralin-1-yl]phenyl]piperidine-4-carbaldehyde (90 mg, 180.9 μmol, 1 eq), AcONa (45 mg, 543 μmol, 3 eq) was added in the DCM (2 mL) and MeOH (2 mL), the mixture was stirred at 30° C. for 1 h, and then AcOH (33 mg, 542 μmol, 31 μL, 3 eq) and Na(OAc)₃BH (77 mg, 362 μmol, 2 eq) was added to the mixture and stirred at 30° C. for 12 h. The reaction mixture was treated with 30 mL H₂O and extracted with 50 mL ethyl acetate. The organic layers were dried with anhydrous Na₂SO₄, filtered, concentrated to give a residue that was purified by prep. HPLC (column: Xtimate C18 150*40 mm*10 um; mobile phase: [water(FA)-ACN]; gradient: 28%-58% B over 6 min). The aqueous phase was lyophilized to dryness to give (38)-3-[5-[4-[[1-[4-[(1*S, 2*R)-2-(2,5-difluoro-4-methyl-phenyl)-4,4-difluoro-6-hydroxy-tetralin-1-yl]phenyl]-4-piperidyl]methyl]piperazin-1-yl]-1-oxo-isoindolin-2-yl]piperidine-2,6-dione (51.4 mg, 63.47 μmol, 35.08% yield). LC-MS (ESI+) m/z: 810.2 (M+H)⁺.

HPLC: 100%, purity at 220 nm.

¹H NMR (400 MHZ, DMSO-d₆) δ=11.02-10.91 (m, 1H), 9.86-9.72 (m, 1H), 8.18-8.12 (m, 0.396H), 7.60-7.45 (m, 1H), 7.23-7.13 (m, 1H), 6.99 (d, J=2.4 Hz, 3H), 6.95-6.79 (m, 2H), 6.70-6.56 (m, 2H), 6.35-6.07 (m, 3H), 5.14-4.93 (m, 1H), 4.39-4.15 (m, 3H), 3.85-3.72 (m, 1H), 3.64-3.50 (m, 2H), 3.32-3.21 (m, 8H), 2.99-2.85 (m, 1H), 2.78-2.62 (m, 2H), 2.44-2.25 (m, 4H), 2.19 (s, 5H), 2.05-1.88 (m, 1H), 1.83-1.54 (m, 3H), 1.26-1.01 (m, 2H).

### Example 37. Preparation of (1-325) (S)-3-(5-(4-((1-(4-((1S,2R)-2-(2,5-difluoro-4-methylphenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

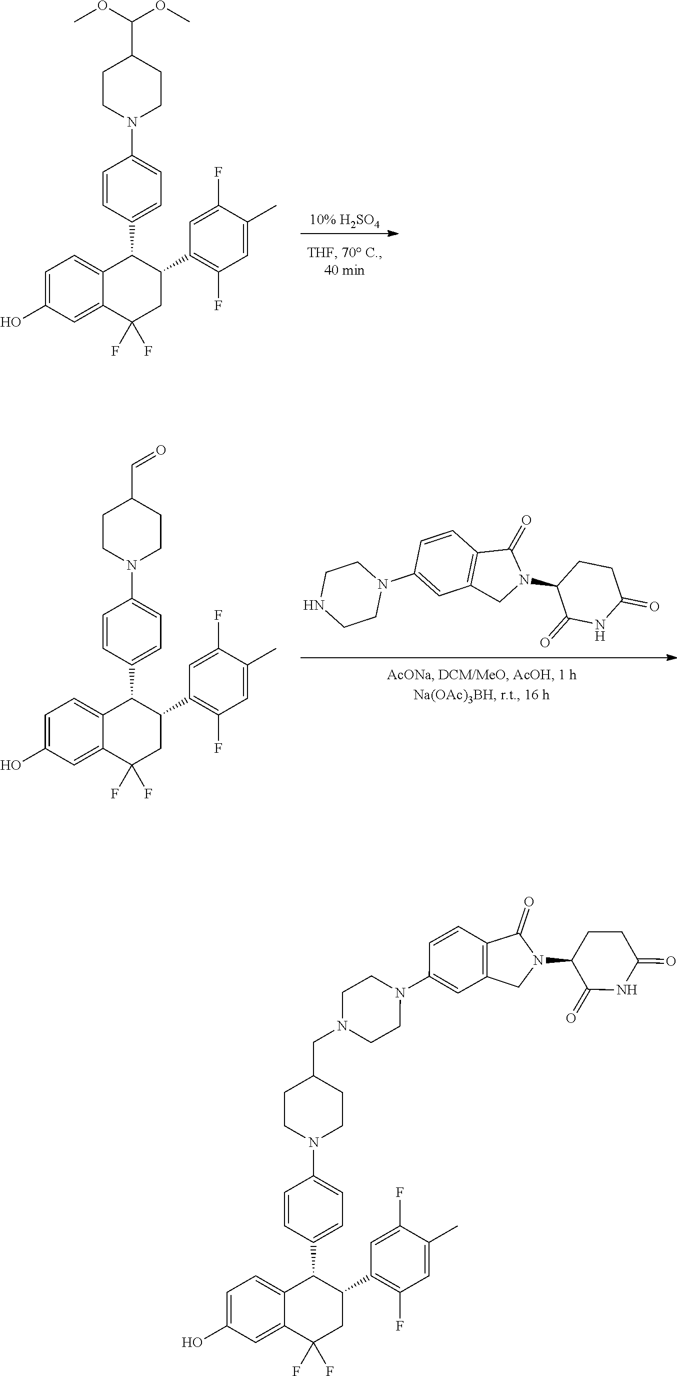 [SMILES: COC(OC)C1CCN(c2ccc([C@H]3c4ccc(O)cc4C(F)(F)C[C@H]3c3cc(F)c(C)cc3F)cc2)CC1 ; Cc1cc(F)c([C@@H]2CC(F)(F)c3cc(O)ccc3[C@@H]2c2ccc(N3CCC(C=O)CC3)cc2)cc1F ; O=C1CC[C@H](N2Cc3cc(N4CCNCC4)ccc3C2=O)C(=O)N1 ; Cc1cc(F)c([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)cc1F]

Step 1: A mixture of 1R,2S)-2-(2,5-difluoro-4-methyl-phenyl)-1-[4-[4-(dimethoxymethyl)-1-piperidyl]phenyl]-4,4-difluoro-tetralin-6-ol (100 mg, 183.9 μmol, 1 eq) was added in THF (3 mL) and 10% H₂SO₄ in water (3 mL) and then the mixture was stirred at 70° C. for 40 min. The resulting solution was quenched with 10 mL aq. NaHCO₃. The mixture was treated with 50 mL H₂O and was extracted with 100 mL ethyl acetate. The organic layers were dried with anhydrous Na₂SO₄, filtered, and concentrated to give 1-[4-[(1R,2S)-2-(2,5-difluoro-4-methyl-phenyl)-4,4-difluoro-6-hydroxy-tetralin-1-yl]phenyl]piperidine-4-carbaldehyde (70 mg, 140.7 μmol, 76% yield).

LC-MS (ESI+) m/z: 498.1 (M+H)+

Step 2: A mixture of (S)-3-(1-oxo-5-(piperazin-1-yl)isoindolin-2-yl)piperidine-2,6-dione (106 mg, 211.05 μmol, 1.5 eq, PhSO₃H salt), 1-[4-[(1R,2S)-2-(2,5-difluoro-4-methyl-phenyl)-4,4-difluoro-6-hydroxy-tetralin-1-yl]phenyl]piperidine-4-carbaldehyde (70 mg, 140.7 μmol, 1 eq), and AcONa (35 mg, 422 μmol, 3 eq) was added in the DCM (2 mL) and MeOH (2 mL), the mixture was stirred at 30° C. for 1 h, and then AcOH (25 mg, 422.09 μmol, 24 μL, 3 eq), Na(OAc)₃BH (60 mg, 281.4 μmol, 2 eq) was added in the mixture and was stirred at 30° C. for 16 h. The reaction mixture was treated with 30 mL H₂O and extracted with 50 mL ethyl acetate. The organic layers were dried with anhydrous Na₂SO₄, filtered, the residue was purified by prep. HPLC (column: Xtimate C18 150*40 mm*10 um; mobile phase: [water(FA)-ACN]; gradient: 28%-58% B over 6 min). The aqueous phase was lyophilized to dryness to give (3S)-3-[5-[4-[[1-[4-[(1*R, 2*S)-2-(2,5-difluoro-4-methyl-phenyl)-4,4-difluoro-6-hydroxy-tetralin-1-yl]phenyl]-4-piperidyl]methyl]piperazin-1-yl]-1-oxo-isoindolin-2-yl]piperidine-2,6-dione (39.3 mg, 48.53 μmol, 34% yield). LC-MS (ESI+) m/z: 810.3 (M+H)⁺.

HPLC: 100%, purity at 220 nm.

¹H NMR (400 MHZ, DMSO-d₆) δ=11.00-10.93 (m, 1H), 9.83-9.74 (m, 1H), 8.17-8.13 (m, 0.334H), 7.55-7.47 (m, 1H), 7.22-7.13 (m, 1H), 7.12-7.01 (m, 3H), 6.93-6.82 (m, 2H), 6.71-6.56 (m, 2H), 6.33 (d, J=4.1 Hz, 3H), 5.05 (dd, J=5.1, 13.1 Hz, 1H), 4.38-4.16 (m, 3H), 3.83-3.73 (m, 1H), 3.64-3.51 (m, 2H), 3.32-3.19 (m, 8H), 2.97-2.82 (m, 1H), 2.78-2.60 (m, 2H), 2.46-2.24 (m, 4H), 2.23-2.11 (m, 5H), 2.03-1.90 (m, 1H), 1.83-1.59 (m, 3H), 1.26-1.07 (m, 2H).

### Example 38. Preparation of (I-315) (S)-3-(5-(4-((1-(4-((1S,2R)-2-(3,4-difluorophenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione and (I-314) (S)-3-(5-(4-((1-(4-((1R,2S)-2-(3,4-difluorophenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

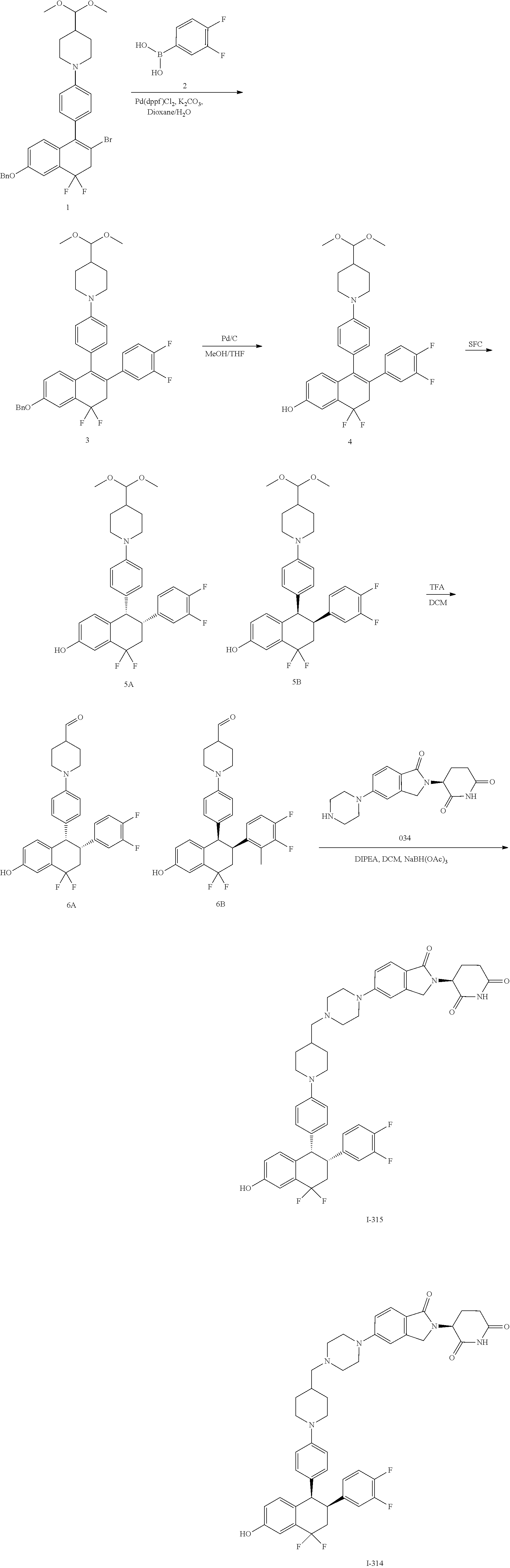

Step 1: To a mixture of compound 1 (618 mg, 1.06 mmol, 1.0 eq) and compound 2 (166.96 mg, 1.06 mmol, 1.0 eq) in 1,4-Dioxane (8 mL) and water (2 mL) was added Pd(dtbpf)Cl₂ (68.91 mg, 105.73 μmol, 0.1 eq), K₂CO₃ (292.26 mg, 2.11 mmol, 2.0 eq) in one portion at 25° C. under N₂. The mixture was stirred for 0.5 hours. The reaction mixture extracted with EtOAc (20 mL×3). The combined organic layers were washed with solvent 10 mL saturated NaCl solution, dried over Na₂SO₄, filtered and concentrated under reduced pressure to give a residue. The residue was purified by prep-HPLC (column: Boston Green ODS 150*30 mm*5 um; mobile phase: water (FA)-ACN; B %: 80%-100%, 12 min) to give product. Compound 3 (200 mg, 323.80 μmol, 30.62% yield) was obtained as a white solid.

LC-MS (ESI⁺) m/z: 618.4 (M+H)⁺.

Step 2: To a solution of compound 3 (230 mg, 372.37 μmol, 1.0 eq) in THF (4 mL) and MeOH (4 mL) was added Pd/C (198.14 mg, 186.18 μmol, 10 wt % purity, 0.5 eq). The suspension was degassed under vacuum and purged with H₂ several times. The mixture was heated to 50° C. (15 psi) and stirred for 6 hours. LCMS showed that the desired compound was detected. The crude product was filtered and the filter was concentrated to give the crude as a yellow solid. The mixture was filtered and concentrated in vacuum. The residue was purified by pre-HPLC (Column Boston Green ODS 150*30 mm*5 um; Condition water(FA)-ACN; B %: 10%-40%, 12 min) to afford compound 4 (130 mg, 245.48 μmol, 65.93% yield) as a white solid.

LC-MS (ESI+) m/z: 530.3 (M+H)⁺.

Step 3: Compound 4 (100 mg) was separated by chiral SFC(Column DAICEL CHIRALPAK AD (250 mm*30 mm, 10 um), Condition CO₂-EtOH (0.1% NH₃H₂O) Begin B 25% End B 25%), FlowRate (ml/min): 60) Compound 5A (35 mg), compound 5B (30 mg).

Step 4: Compound 6A: To a solution of compound 5A (20 mg, 37.77 μmol, 1.0 eq) in DCM (2 mL) was added TFA (4.31 mg, 37.77 μmol, 1.0 eq). The mixture was stirred at 25° C. for 0.2 hr. LCMS showed that the starting material was consumed and the desired product was detected. The reaction liquid was dried with N₂ at 0° C. to give Compound 6A (13.5 mg, 27.92 μmol, 73.93% yield) was obtained as yellow oil which was used in the next step without further purification.

LC-MS (ESI+) m/z: 484.3 (M+H)⁺.

Step 5: Compound 6B: To a solution of compound 5B (25 mg, 47.21 μmol, 1.0 eq) in DCM (2 mL) was added TFA (5.38 mg, 47.21 μmol, 1.0 eq). The mixture was stirred at 25° C. for 0.2 hr. LCMS showed that the starting material was consumed and the desired product was detected. The reaction liquid is dried with N₂ at 0° C. to give Compound 6B (18 mg, 37.23 μmol, 78.86% yield) was obtained as yellow oil which was used in the next step without further purification.

LC-MS (ESI+) m/z: 484.3 (M+H)⁺.

Step 6: To a solution of compound 6A (13.5 mg, 27.92 μmol, 1.0 eq) in DCM (mb) and MeOH (3 mL) was added compound 034 (9.17 mg, 27.92 μmol, 1.0 eq) and DIEA (3.61 mg, 27.92 μmol, 1.0 eq) at 25° C. After addition, the mixture was stirred at this temperature for 0.5 hr, and then NaBH(OAc); (11.84 mg, 55.84 μmol, 2.0 eq) was added at 25° C. The resulting mixture was stirred at 25° C. for 1 hr. The reaction mixture was concentrated to yield a residue. The residue was purified by preparative HPLC (column: Boston Green ODS 150*30 mm*5 um; mobile phase: [water(FA)-ACN]; B %: 27%-37%, 12 min), followed by lyophilization to yield I-315 (12.4 mg, 15.58 μmol, 55.80% yield) was obtained as a white solid. LC-MS (ESI+) m/z: 796.5 [M+H]⁺.

LCMS: calc. for C₄₅H₄₅F₄N₅O₄: 795.34, found: [M+H]⁺ 796.5.

HPLC: 98.58% purity at 220 nm.

¹H NMR (400 MHz, METHANOL-d₄) δ ppm 7.65 (d, J=8.58 Hz, 1H), 7.04-7.16 (m, 4H), 6.83-6.93 (m, 2H), 6.64-6.76 (m, 4H), 6.33 (d, J=8.70 Hz, 2H), 5.11 (dd, J=13.29, 5.07 Hz, 1H), 4.36-4.48 (m, 2H), 4.32 (br d, J=3.46 Hz, 1H), 3.69-3.77 (m, 1H), 3.57-3.63 (m, 2H), 3.37-3.40 (m, 4H), 2.87-2.97 (m, 1H), 2.67-2.84 (m, 2H), 2.61-2.66 (m, 5H), 2.41-2.54 (m, 1H), 2.31 (d, J=7.03 Hz, 2H), 2.13-2.20 (m, 1H), 1.89 (br d, J=12.16 Hz, 2H), 1.62-1.75 (m, 1H), 1.31-1.35 (m, 4H).

Step 7: To a solution of compound 6B (10 mg, 20.68 μmol, 1.0 eq) in DCM (3 mL) and MeOH (3 mL) was added compound 034 (6.79 mg, 20.68 μmol, 1.0 eq) and DIEA (2.67 mg, 20.68 μmol, 1.0 eq) at 25° C. After addition, the mixture was stirred at this temperature for 0.5 hr, and then NaBH(OAc); (8.77 mg, 41.37 μmol, 2.0 eq) was added at 25° C. The resulting mixture was stirred at 25° C. for 1 hr. The reaction mixture was concentrated to yield a residue. The residue was purified by preparative HPLC (column: Boston Green ODS 150*30 mm*5 um; mobile phase: [water(FA)-ACN]; B %: 27%-37%, 12 min), followed by lyophilization to yield I-314 (6.7 mg, 8.42 μmol, 40.70% yield) was obtained as a white solid. LC-MS (ESI+) m/z: 796.5 [M+H]⁺.

LCMS: calc. for C₄₅H₄₅F₄NO₄: 795.34, found: [M+H]⁺ 796.5.

HPLC: 97.25% purity at 220 nm

¹H NMR (400 MHZ, METHANOL-d₄) δ ppm 7.63 (d, J=8.46 Hz, 1H), 7.01-7.17 (m, 4H), 6.80 (s, 2H), 6.61-6.74 (m, 4H), 6.26-6.38 (m, 2H), 5.09 (dd, J=13.41, 5.30 Hz, 1H), 4.40 (d, J=6.08 Hz, 2H), 4.29 (br d, J=2.86 Hz, 1H), 3.68-3.75 (m, 1H), 3.58 (br d, J=11.92 Hz, 2H), 3.34-3.38 (m, 4H), 2.84-2.94 (m, 1H), 2.65-2.82 (m, 2H), 2.59-2.64 (m, 5H), 2.40-2.51 (m, 1H), 2.29 (br d, J=7.03 Hz, 2H), 2.13-2.20 (m, 1H), 1.79-1.93 (m, 2H), 1.60-1.73 (m, 1H), 1.29-1.33 (m, 4H).

### Example 39. Preparation of (I-337) (S)-3-(5-(4-((1-(6-((1R,2R)-4,4-difluoro-6-hydroxy-2-phenyl-1,2,3,4-tetrahydronaphthalen-1-yl) pyridin-3-yl)piperidin-4-yl)methyl)piperazin-1-yl)-1-oxoisoindolin-2-yl)piperidine-2,6-dione

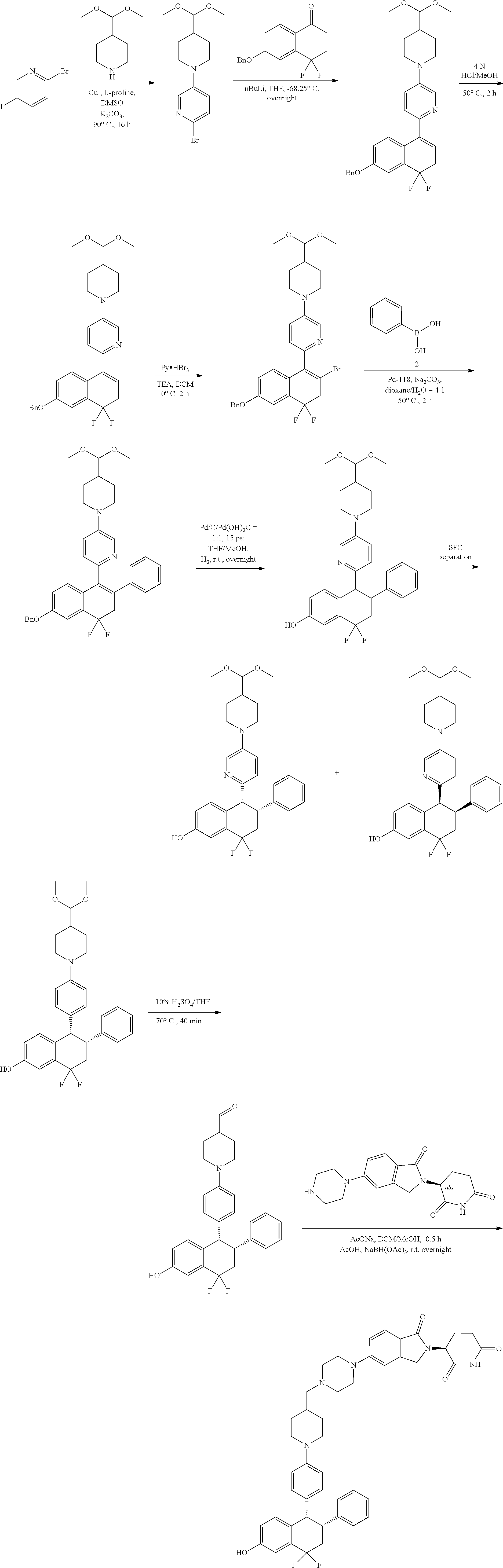

Step 1: A mixture of 4-(dimethoxymethyl)piperidine (2.8 g, 17.6 mmol, 1 eq), 2-bromo-5-iodo-pyridine (5 g, 17.6 mmol, 1 eq), iodocopper (671 mg, 3.5 mmol, 0.2 eq), dipotassium carbonate (4.9 g, 35 mmol, 2 eq), (2S)-pyrrolidine-2-carboxylic acid (811 mg, 7.04 mmol, 0.4 eq) was added in DMSO (50 mL) and purged with N₂ for 3 times, the mixture was stirred at 90° C. for 16 h under N₂. TLC (petroleum ether:ethyl acetate=5:1, R_{f}=0.7, UV) showed one main new spot was observed. Then the mixture was treated with H₂O (50 mL), extracted with ethyl acetate (100 mL). The combined extracts was dried over anhydrous Na₂SO₄, filtered and concentrated to dryness in vacuum to give a residue. The residue was purified by flash silica gel chromatography (ISCO®; 40 g SepaFlash® Silica Flash Column, Eluent of 0˜20% Petroleum ether/Ethyl acetate @ 40 mL/min). The pure fractions were collected and concentrated to dryness in vacuum to give 2-bromo-5-(4-(dimethoxymethyl)piperidin-1-yl)pyridine (1.6 g, 5.1 mmol, 29% yield) was obtained as a yellow oil. LC-MS (ESI+) m/z: 317.1 (M+H)+

Step 2: A mixture of butyllithium (2.5 M, 2.4 mL, 1.2 eq) was added into mixture of 6-(benzyloxy)-4,4-difluoro-3,4-dihydronaphthalen-1 (2H)-one (2.9 g, 10.2 mmol, 2 eq) in THF (15 mL) under N₂ at −78° C., and the mixture was stirred at −68° C. for 0.5 h under N₂ atmosphere, and then 2-bromo-5-(4-(dimethoxymethyl)piperidin-1-yl)pyridine (1.6 g, 5.08 mmol, 1 eq) was added in the mixture at −68° C. and was stirred at 25° C. for 16 h. TLC (petroleum ether:ethyl acetate=5:1, R_{f}=0.5 UV) showed one main new spot was observed. Then the mixture was quenched with saturation NH₄Cl 20 ml at 0° C. under N₂ and treated with H₂O (50 mL), extracted with ethyl acetate (100 mL). The combined organic layers were washed with brine (100 mL) and was dried over anhydrous Na₂SO₄, filtered and concentrated to dryness in vacuum to give a residue. The residue was purified by flash silica gel chromatography (ISCO®; 40 g SepaFlash® Silica Flash Column, Eluent of 0˜10% Petroleum ether/Ethyl acetate @ 40 mL/min). The pure fractions were collected and concentrated to dryness in vacuum to give 6-(benzyloxy)-1-(5-(4-(dimethoxymethyl)piperidin-1-yl) pyridin-2-yl)-4,4-difluoro-1,2,3,4-tetrahydronaphthalen-1-ol (1.8 g, 3.4 mmol, 66.8% yield) was obtained as a yellow oil. LC-MS (ESI+) m/z: 525.2 (M+H)+

Step 3: A mixture of 6-(benzyloxy)-1-(5-(4-(dimethoxymethyl)piperidin-1-yl) pyridin-2-yl)-4,4-difluoro-1,2,3,4-tetrahydronaphthalen-1-ol (1.7 g, 3.2 mmol, 1 eq) was added in 4 N HCl in MeOH (10 mL), the mixture was stirred at 50° C. for 2 h. The reaction mixture was filtered and concentrated to dryness in vacuo to give 2-(6-(benzyloxy)-4,4-difluoro-3,4-dihydronaphthalen-1-yl)-5-(4-(dimethoxymethyl)piperidin-1-yl)pyridine (910 mg, 1.8 mmol, 55% yield).

Step 4: A mixture of 2-(6-(benzyloxy)-4,4-difluoro-3,4-dihydronaphthalen-1-yl)-5-(4-(dimethoxymethyl)piperidin-1-yl)pyridine (910 mg, 1.8 mmol, 1 eq), N,N-diethylethanamine (273 mg, 2.7 mmol, 375 μL, 1.5 eq), Py·HBr₃ (747 mg, 2.3 mmol, 1.3 eq) in DCM (30 mL) was degassed at 0° C. for 2 h. TLC (petroleum ether:ethyl acetate=5:1, R_{f}=0.6 UV) showed one main new spot was observed. The resulting solution was diluted with H₂O (50 mL) and extracted with DCM (100 mL×3). The organic layers were dried with anhydrous Na₂SO₄, filtered, and concentrated to give the crude product. The residue was purified by flash silica gel chromatography (ISCO®; 40 g SepaFlash® Silica Flash Column, Eluent of 0˜30% Petroleum ether/Ethyl acetate @ 30 mL/min). The pure fractions were collected and concentrated to dryness in vacuo to give 2-(6-(benzyloxy)-2-bromo-4,4-difluoro-3,4-dihydronaphthalen-1-yl)-5-(4-(dimethoxymethyl)piperidin-1-yl)pyridine (920 mg, 1.6 mmol, 87% yield).

Step 5: A mixture of 2-(6-(benzyloxy)-2-bromo-4,4-difluoro-3,4-dihydronaphthalen-1-yl)-5-(4-(dimethoxymethyl)piperidin-1-yl)pyridine (820 mg, 1.4 mmol, 1 eq), phenylboronic acid (205 mg, 1.7 mmol, 1.2 eq), Na₂CO₃ (445 mg, 4.2 mmol, 3 eq), Pd-118 (91 mg, 140 μmol, 0.1 eq) in dioxane (10 mL) and H₂O (2.5 mL) was degassed and purged with N₂ for 3 times, and then the mixture was stirred at 50° C. for 2 h under N₂ atmosphere. TLC (petroleum ether:ethyl acetate=5:1, R_{f}=0.5 UV) showed one main new spot was observed. Then the mixture was treated with H₂O (50 mL), extracted with ethyl acetate (100 mL). The combined organic layers were washed with saturated salt water (100 mL) and was dried over anhydrous Na₂SO₄, filtered and concentrated to dryness in vacuum to give a residue. The residue was purified by flash silica gel chromatography (ISCOR; 20 g SepaFlash® Silica Flash Column, Eluent of 0˜10% Petroleum ether/Ethyl acetate @ 20 mL/min). The pure fractions were collected and concentrated to dryness in vacuum to give 2-(6-(benzyloxy)-4,4-difluoro-2-phenyl-3,4-dihydronaphthalen-1-yl)-5-(4-(dimethoxymethyl)piperidin-1-yl)pyridine (800 mg, 1.4 mmol, 98% yield) was obtained as a yellow oil.

Step 6: A mixture of 2-(6-(benzyloxy)-4,4-difluoro-2-phenyl-3,4-dihydronaphthalen-1-yl)-5-(4-(dimethoxymethyl)piperidin-1-yl)pyridine (170 mg, 292 μmol, 1 eq), Pd/C (90 mg, 85 μmol, 10% purity, 0.29 eq), Pd(OH)₂/C (90 mg, 641 μmol, 2.2 eq) was added in THE (5 mL) and MeOH (5 mL) under N₂ atmosphere, the suspension was degassed and purged with H₂ for 3 times. The mixture was stirred under H₂ (15 psi) at 25° C. overnight. The reaction mixture was filtered and concentrated under reduced pressure to give a residue. The crude product was purified by reversed-phase SFC(Column: DAICEL CHIRALCEL OJ (250 mm*30 mm, 10 um); Condition: CO₂-EtOH (0.1% NH; in H₂O); at the beginning: B (45%); at the end: B (45%); Flow Rate (ml/min) δ0. The reaction mixture was filtered and concentrated to dryness in vacuum to give (5R,6R)-5-(5-(4-(dimethoxymethyl)piperidin-1-yl) pyridin-2-yl)-8,8-difluoro-6-phenyl-5,6,7,8-tetrahydronaphthalen-2-ol (30 mg, 59.9 μmol), (5S,6S)-5-(5-(4-(dimethoxymethyl)piperidin-1-yl) pyridin-2-yl)-8,8-difluoro-6-phenyl-5,6,7,8-tetrahydronaphthalen-2-ol (30 mg, 59.9 μmol) was obtained as a white solid. LC-MS (ESI+) m/z: 494.58 (M+H)⁺

Step 7: A mixture of (5R, 6R)-5-(5-(4-(dimethoxymethyl)piperidin-1-yl) pyridin-2-yl)-8,8-difluoro-6-phenyl-5,6,7,8-tetrahydronaphthalen-2-ol (30 mg, 59.9 μmol, 1 eq) was added THF (2 mL) and 10% H₂SO₄ (2 mL). The mixture was stirred at 70° C. for 40 min. The resulting solution was quenched with 10 mL aq. NaHCO₃ and was treated with 20 mL H₂O and was extracted with ethyl acetate (50 mL×3). The organic layers were dried with anhydrous Na₂SO₄, filtered, and concentrated to give the crude product. The reaction mixture was filtered and concentrated to dryness in vacuum to give 1-(6-((1R,2R)-4,4-difluoro-6-hydroxy-2-phenyl-1,2,3,4-tetrahydronaphthalen-1-yl) pyridin-3-yl)piperidine-4-carbaldehyde (25 mg, 55 μmol, 92% yield).

Step 8: A mixture of (S)-3-(1-oxo-5-(piperazin-1-yl)isoindolin-2-yl)piperidine-2,6-dione (41 mg, 82.50 μmol, 1.5 eq, PhSO₃H salt), 1-(6-((1R,2R)-4,4-difluoro-6-hydroxy-2-phenyl-1,2,3,4-tetrahydronaphthalen-1-yl) pyridin-3-yl)piperidine-4-carbaldehyde (25 mg, 55 μmol, 1 eq), AcONa (14 mg, 165 μmol, 3 eq) was added in the DCM (2 mL) and MeOH (2 mL), the mixture was stirred at 30° C. for 0.5 h, and then AcOH (10 mg, 165 μmol, 9 μL, 3 eq), NaBH(OAc); (23 mg. 110 μmol, 2 eq) was added in the mixture and was stirred at 30° C. for 16 h. The reaction mixture was treated with 30 mL H₂O and extracted with 50 mL ethyl acetate. The organic layers were dried with anhydrous Na₂SO₄, filtered, the residue was purified by prep. HPLC (column: Welch Xtimate C18 150*30 mm*5 um; mobile phase: [water(FA)-ACN]; gradient: 12%-42% B over 7 min). The aqueous phase was lyophilized to dryness to give (S)-3-(5-(4-((1-(6-((1R,2R)-4,4-difluoro-6-hydroxy-2-phenyl-1,2,3,4-tetrahydronaphthalen-1-yl) pyridin-3-yl)piperidin-4-yl)methyl)piperazin-1-yl)-1-oxoisoindolin-2-yl)piperidine-2,6-dione (9.3 mg, 12.2 μmol, 22% yield). LC-MS (ESI+) m/z: 761.2 (M+H)⁺.

HPLC: 100%, purity at 220 nm.

¹H NMR (400 MHZ, DMSO-d₆) δ=10.99-10.94 (m, 1H), 9.66-9.59 (m, 1H), 8.16 (s, 0.295H), 8.09-8.00 (m, 1H), 7.57-7.46 (m, 1H), 7.22-7.00 (m, 6H), 6.97-6.75 (m, 5H), 6.47-6.33 (m, 1H), 5.10-5.00 (m, 1H), 4.39-4.28 (m, 2H), 4.25-4.16 (m, 1H), 3.75-3.46 (m, 4H), 3.32-3.21 (m, 5H), 2.99-2.80 (m, 1H), 2.70-2.53 (m, 5H), 2.44-2.14 (m, 5H), 2.01-1.88 (m, 1H), 1.82-1.60 (m, 3H), 1.28-1.04 (m, 2H).

### Example 40. Preparation of (1-203) (S)-3-(5-(4-((1-(6-((1S,2S)-4,4-difluoro-6-hydroxy-2-phenyl-1,2,3,4-tetrahydronaphthalen-1-yl) pyridin-3-yl)piperidin-4-yl)methyl)piperazin-1-yl)-1-oxoisoindolin-2-yl)piperidine-2,6-dione

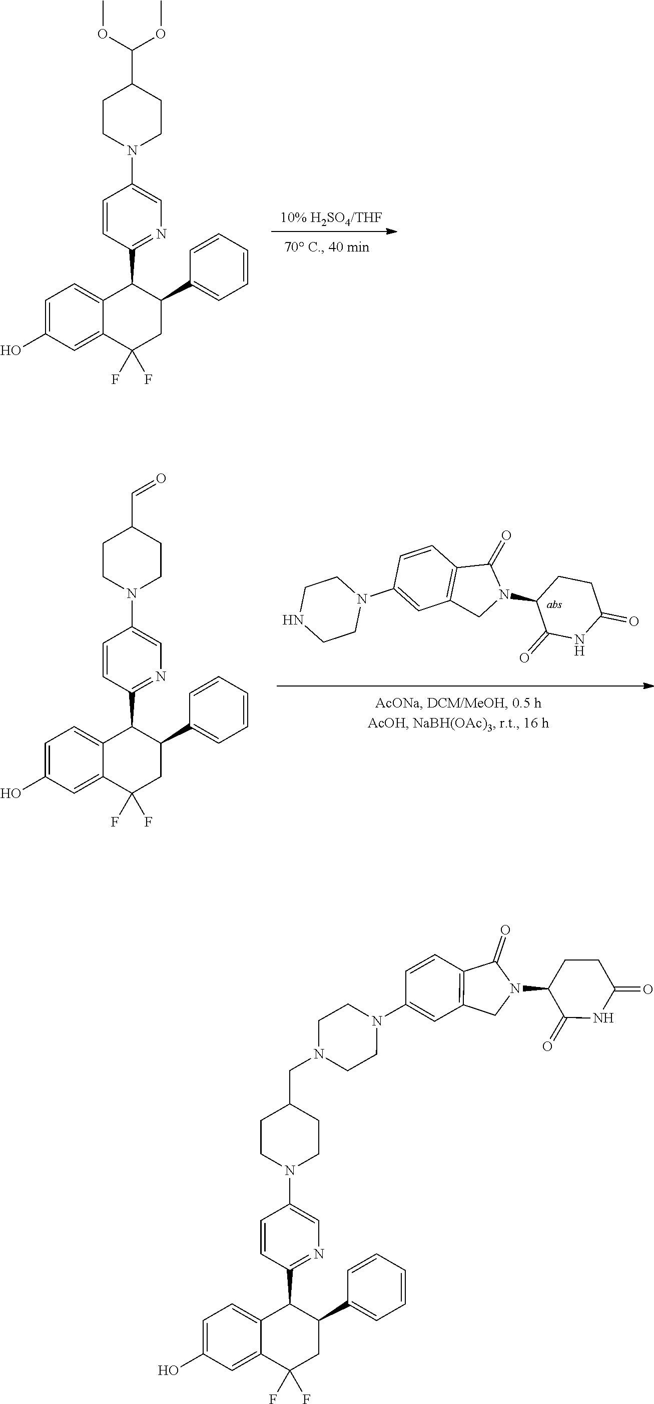 [SMILES: COC(OC)C1CCN(c2ccc([C@@H]3c4ccc(O)cc4C(F)(F)C[C@@H]3c3ccccc3)nc2)CC1 ; O=CC1CCN(c2ccc([C@@H]3c4ccc(O)cc4C(F)(F)C[C@@H]3c3ccccc3)nc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccccc7)nc6)CC5)CC4)ccc3C2=O)C(=O)N1]

Step 1: A mixture of (5S,6S)-5-(5-(4-(dimethoxymethyl)piperidin-1-yl) pyridin-2-yl)-8,8-difluoro-6-phenyl-5,6,7,8-tetrahydronaphthalen-2-ol (30 mg, 60 μmol, 1 eq) was added THF (2 mL) and 10% H₂SO₄ (2 mL). The mixture was stirred at 70° C. for 40 min. The resulting solution was quenched with 10 ml NaHCO₃ and was treated with 20 mL H₂O and was extracted with ethyl acetate (50 mL×3). The organic layers were dried with anhydrous Na₂SO₄, filtered, and concentrated to give the crude product. The reaction mixture was filtered and concentrated to dryness in vacuum to give 1-(6-((1S,2S)-4,4-difluoro-6-hydroxy-2-phenyl-1,2,3,4-tetrahydronaphthalen-1-yl) pyridin-3-yl)piperidine-4-carbaldehyde (25 mg, 55 μmol, 92% yield).

Step 2: A mixture of (S)-3-(1-oxo-5-(piperazin-1-yl)isoindolin-2-yl)piperidine-2,6-dione (41 mg, 82.5 μmol, 1.5 eq, PhSO₃H salt), 1-(6-((1S,2S)-4,4-difluoro-6-hydroxy-2-phenyl-1,2,3,4-tetrahydronaphthalen-1-yl) pyridin-3-yl)piperidine-4-carbaldehyde (25 mg, 55 μmol, 1 eq), AcONa (14 mg, 165 μmol, 3 eq) was added in the DCM (2 mL) and MeOH (2 mL), the mixture was stirred at 30° C. for 0.5 h, and then AcOH (10 mg, 165 μmol, 9 μL, 3 eq), NaBH(OAc); (23 mg, 110 μmol, 2 eq) was added in the mixture and was stirred at 30° C. for 16 h. The reaction mixture was treated with 30 mL H₂O and extracted with 50 mL ethyl acetate. The organic layers were dried with anhydrous Na₂SO₄, filtered, the residue was purified by prep. HPLC (column: Welch Xtimate C18 150*30 mm*5 um; mobile phase: [water(FA)-ACN]; gradient: 12%-42% B over 7 min). The aqueous phase was lyophilized to dryness to give (3S)-3-[5-[4-[[-1-[6-[(1S,2S)-4,4-difluoro-6-hydroxy-2-phenyl-tetralin-1-yl]-3-pyridyl]-4-piperidyl]methyl]piperazin-1-yl]-1-oxo-isoindolin-2-yl]piperidine-2,6-dione (12.1 mg, 15.9 μmol, 28% yield). LC-MS (ESI+) m/z: 761.1 (M+H)⁺.

HPLC: 100%, purity at 220 nm.

¹H NMR (400 MHZ, DMSO-d₆) δ=10.99-10.93 (m, 1H), 9.66-9.62 (m, 1H), 8.18-8.14 (m, 0.307H), 8.09-8.01 (m, 1H), 7.56-7.48 (m, 1H), 7.19-7.10 (m, 3H), 7.09-7.02 (m, 3H), 6.96-6.89 (m, 3H), 6.87-6.75 (m, 2H), 6.44-6.36 (m, 1H), 5.10-5.01 (m, 1H), 4.39-4.29 (m, 2H), 4.25-4.16 (m, 1H), 3.75-3.48 (m, 4H), 3.33-3.21 (m, 5H), 3.00-2.85 (m, 1H), 2.70-2.54 (m, 5H), 2.45-2.15 (m, 5H), 2.04-1.90 (m, 1H), 1.81-1.61 (m, 3H), 1.25-1.03 (m, 2H).

### Example 41. Preparation of (1-330) (S)-3-(5-(4-((1-(4-((1S,2R)-4,4-difluoro-6-hydroxy-2-(4-(trifluoromethyl)phenyl)-1,2,3,4-tetrahydronaphthalen-1-yl)phenyl)piperidin-4-yl)methyl)piperazin-1-yl)-1-oxoisoindolin-2-yl)piperidine-2,6-dione

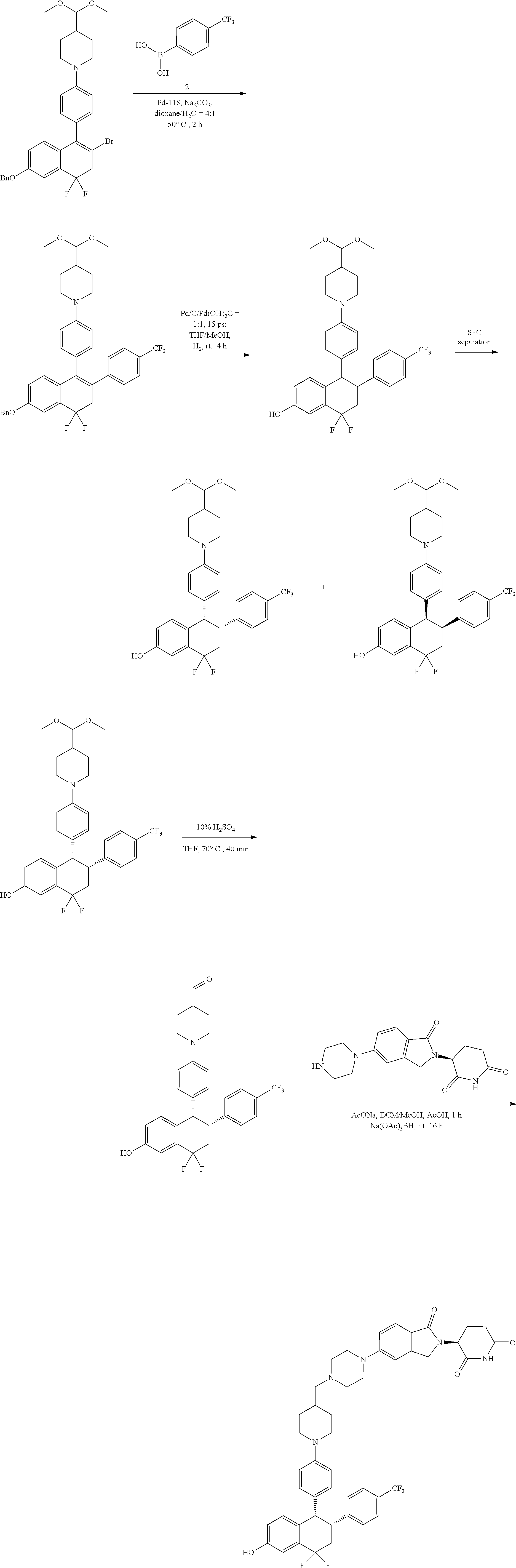

Step 1: A mixture of 1-(4-(6-(benzyloxy)-2-bromo-4,4-difluoro-3,4-dihydronaphthalen-1-yl)phenyl)-4-(dimethoxymethyl)piperidine (600 mg, 1 mmol, 1 eq), [4-(trifluoromethyl)phenyl]boronic acid (234 mg, 1.2 mmol, 1.2 eq), Na₂CO₃ (326 mg, 3 mmol, 3 eq), Pd-118 (67 mg, 103 μmol, 0.1 eq) in dioxane (8 mL) and H₂O (2 mL) was degassed and purged with N₂ for 3 times, and then the mixture was stirred at 50° C. for 2 h under N₂ atmosphere. TLC (petroleum ether:ethyl acetate=5:1, R_{f}=0.5 UV) showed one main new spot was observed. The resulting solution was treated with 50 mL H₂O and was extracted with 100 mL ethyl acetate. The organic layers were dried with anhydrous Na₂SO₄, filtered, and concentrated to give the crude product. The residue was purified by column chromatography (SiO₂, Petroleum ether/Ethyl acetate=10/0 to 8/2). The reaction mixture was filtered and concentrated to dryness in vacuum to give 1-(4-(6-(benzyloxy)-4,4-difluoro-2-(4-(trifluoromethyl)phenyl)-3,4-dihydronaphthalen-1-yl)phenyl)-4˜(dimethoxymethyl)piperidine (570 mg, 877 μmol, 85% yield) was obtained as a yellow oil.

Step 2: A mixture of 1-(4-(6-(benzyloxy)-4,4-difluoro-2-(4-(trifluoromethyl)phenyl)-3,4-dihydronaphthalen-1-yl)phenyl)-4-(dimethoxymethyl)piperidine (570 mg, 877 μmol, 1 eq), Pd/C (519 mg, 488 μmol, 10% purity), Pd(OH)₂/C (519 mg, 3.7 mmol, 10% purity) was added in THE (5 mL) and MeOH (5 mL) under N₂ atmosphere. The suspension was degassed and purged with H₂ for 3 times. The mixture was stirred under H₂ (15 psi) at 25° C. for 4 h. The reaction mixture was filtered and concentrated under reduced pressure to give a residue. The crude product was purified by reversed-phase SFC (column: DAICEL CHIRALPAK AD (250 mm*30 mm, 10 um); mobile phase: [CO2-i-PrOH (0.1% NH₃H₂O)]; B %: 20% %, isocratic elution mode retention time 1.373, 1.512). The reaction mixture was filtered and concentrated to dryness in vacuum to give (5S,6R)-5-(4-(4-(dimethoxymethyl)piperidin-1-yl)phenyl)-8,8-difluoro-6-(4-(trifluoromethyl)phenyl)-5,6,7,8-tetrahydronaphthalen-2-ol (110 mg, 196 μmol, 22% yield), (5R,6S)-5-(4-(4-(dimethoxymethyl)piperidin-1-yl)phenyl)-8,8-difluoro-6-(4-(trifluoromethyl)phenyl)-5,6,7,8-tetrahydronaphthalen-2-ol (120 mg, 214 μmol, 24% yield) was obtained as a white solid. LC-MS (ESI+) m/z: 562.1 (M+H)+

Step 3: A mixture of (5S,6R)-5-(4-(4-(dimethoxymethyl)piperidin-1-yl)phenyl)-8,8-difluoro-6-(4-(trifluoromethyl)phenyl)-5,6,7,8-tetrahydronaphthalen-2-ol (110 mg, 196 μmol, 1 eq) was added THF (3 mL) and 10% H₂SO₄ (3 mL). The mixture was stirred at 70° C. for 40 min. The resulting solution was quenched with 10 mL aq. NaHCO₃, the mixture was treated with 50 mL H₂O and was extracted with 100 mL ethyl acetate. The organic layers were dried with anhydrous Na₂SO₄, filtered, and concentrated to give 1-(4-((1S,2R)-4,4-difluoro-6-hydroxy-2-(4-(trifluoromethyl)phenyl)-1,2,3,4-tetrahydronaphthalen-1-yl)phenyl)piperidine-4-carbaldehyde (100 mg, 194 μmol, 99% yield) was obtained as a white solid.

Step 4: A mixture of (S)-3-(1-oxo-5-(piperazin-1-yl)isoindolin-2-yl)piperidine-2,6-dione (146 mg, 291 μmol, 1.5 eq, PhSO₃H salt), 1-(4-((1S,2R)-4,4-difluoro-6-hydroxy-2-(4-(trifluoromethyl)phenyl)-1,2,3,4-tetrahydronaphthalen-1-yl)phenyl)piperidine-4-carbaldehyde (100 mg, 194 μmol, 1 eq), AcONa (48 mg, 582 μmol, 3 eq) was added in the DCM (2 mL) and MeOH (2 mL), the mixture was stirred at 30° C. for 1 h, and then AcOH (35 mg, 582 μmol, 33 AL, 3 eq), Na(OAc)₃BH (82 mg, 388 μmol, 2 eq) was added in the mixture and was stirred at 30° C. for 16 h. The reaction mixture was treated with 30 mL H₂O and extracted with 50 mL ethyl acetate. The organic layers were dried with anhydrous Na₂SO₄, filtered, the residue was purified by prep. HPLC (column: Welch Xtimate C18 150*30 mm*5 um; mobile phase: [water(FA)-ACN]; gradient: 26%-56% B over 7 min). The aqueous phase was lyophilized to dryness to give (S)-3-(5-(4-((1-(4-((1S,2R)-4,4-difluoro-6-hydroxy-2-(4-(trifluoromethyl)phenyl)-1,2,3,4-tetrahydronaphthalen-1-yl)phenyl)piperidin-4-yl)methyl)piperazin-1-yl)-1-oxoisoindolin-2-yl)piperidine-2,6-dione (68.6 mg, 83 μmol, 43% yield). LC-MS (ESI+) m/z: 828.3 (M+H)⁺.

HPLC: 98.948%, purity at 220 nm.

¹H NMR (400 MHz, DMSO-d₆) δ=11.00-10.90 (m, 1H), 9.89-9.69 (m, 1H), 8.15 (s, 0.41H), 7.60-7.46 (m, 3H), 7.20-7.12 (m, 2H), 7.11-7.01 (m, 3H), 6.92-6.82 (m, 2H), 6.64-6.54 (m, 2H), 6.22-6.12 (m, 2H), 5.11-5.01 (m, 1H), 4.40-4.28 (m, 2H), 4.25-4.15 (m, 1H), 3.86-3.75 (m, 1H), 3.62-3.48 (m, 2H), 3.47-3.33 (m, 8H), 3.00-2.83 (m, 1H), 2.81-2.54 (m, 4H), 2.43-2.28 (m, 2H), 2.28-2.10 (m, 2H), 2.03-1.89 (m, 1H), 1.81-1.57 (m, 3H), 1.03 (s, 2H).

### Example 42. Preparation of (1-329) (S)-3-(5-(4-((1-(4-((1R,2S)-4,4-difluoro-6-hydroxy-2-(4-(trifluoromethyl)phenyl)-1,2,3,4-tetrahydronaphthalen-1-yl)phenyl)piperidin-4-yl)methyl)piperazin-1-yl)-1-oxoisoindolin-2-yl)piperidine-2,6-dione

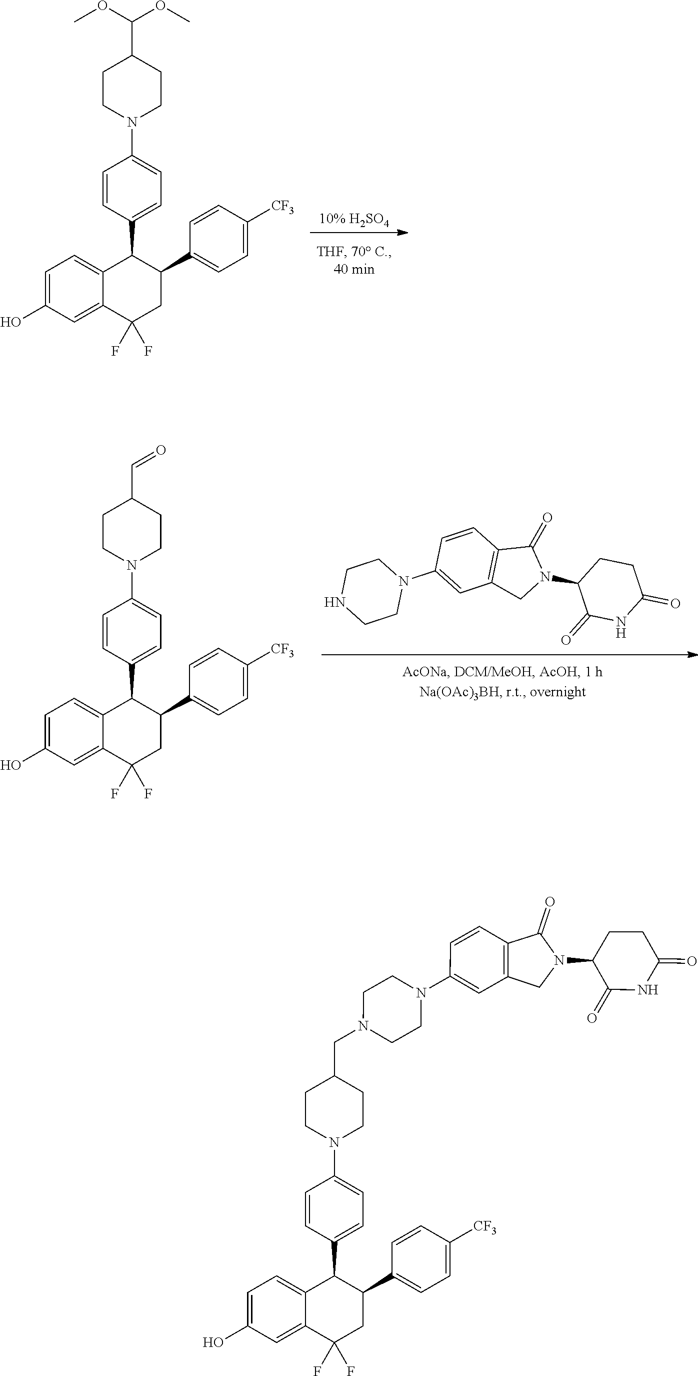 [SMILES: COC(OC)C1CCN(c2ccc([C@@H]3c4ccc(O)cc4C(F)(F)C[C@@H]3c3ccc(C(F)(F)F)cc3)cc2)CC1 ; O=CC1CCN(c2ccc([C@@H]3c4ccc(O)cc4C(F)(F)C[C@@H]3c3ccc(C(F)(F)F)cc3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccc(C(F)(F)F)cc7)cc6)CC5)CC4)ccc3C2=O)C(=O)N1]

Step 1: A mixture of (5R,6S)-5-(4-(4-(dimethoxymethyl)piperidin-1-yl) phenyl)-8,8-difluoro-6-(4-(trifluoromethyl)phenyl)-5,6,7,8-tetrahydronaphthalen-2-ol (120 mg, 213 μmol, 1 eq) was added THF (3 mL) and H₂SO₄ (3 mL). The mixture was stirred at 70° C. for 40 min. The resulting solution was quenched with 10 mL aq. NaHCO₃, the mixture was treated with 50 mL H₂O and was extracted with 50 mL ethyl acetate. The organic layers were dried with anhydrous Na₂SO₄, filtered, and concentrated to give 1-[4-[(1R,2S)-4,4-difluoro-6-hydroxy-2-[4-(trifluoromethyl)phenyl]tetralin-1-yl]phenyl]piperidine-4-carbaldehyde (100 mg, 194 μmol, 91% yield) was obtained as a white solid.

Step 2: A mixture of S-3-(1-oxo-5-(piperazin-1-yl)isoindolin-2-yl)piperidine-2,6-dione (146 mg, 291 μmol, 1.5 eq, PhSO₃H salt), 1-[4-[(1R,2S)-4,4-difluoro-6-hydroxy-2-[4-(trifluoromethyl)phenyl]tetralin-1-yl]phenyl]piperidine-4-carbaldehyde (100 mg, 194 μmol, 1 eq), AcONa (48 mg, 582 μmol, 3 eq) was added in the DCM (2 mL) and MeOH (2 mL), the mixture was stirred at 30° C. for 1 h, and then AcOH (35 mg, 582 μmol, 33 μL, 3 eq), Na(OAc)₃BH (82 mg, 388 μmol, 2 eq) was added in the mixture and was stirred at 30° C. for 16 h. The reaction mixture was treated with 30 mL H₂O and extracted with 50 mL ethyl acetate. The organic layers were dried with anhydrous Na₂SO₄, filtered, the residue was purified by prep. HPLC (column: Welch Xtimate C18 150*30 mm*5 um; mobile phase: [water(FA)-ACN]; gradient: 26%-56% B over 7 min). The aqueous phase was lyophilized to dryness to give (3S)-3-[5-[4-[[1-[4-[(1R,2S)-4,4-difluoro-6-hydroxy-2-[4-(trifluoromethyl)phenyl]tetralin-1-yl]phenyl]-4-piperidyl]methyl]piperazin-1-yl]-1-oxo-isoindolin-2-yl]piperidine-2,6-dione (82.6 mg, 99 μmol, 51% yield). LC-MS (ESI+) m/z: 828.2 (M+H)⁺.

HPLC: 100%, purity at 220 nm.

¹H NMR (400 MHZ, DMSO-d₆) δ=11.03-10.87 (m, 1H), 9.89-9.69 (m, 1H), 8.19-8.10 (m, 0.245H), 7.61-7.44 (m, 3H), 7.20-7.12 (m, 2H), 7.11-6.99 (m, 3H), 6.94-6.81 (m, 2H), 6.68-6.51 (m, 2H), 6.22-6.05 (m, 2H), 5.14-4.97 (m, 1H), 4.44-4.26 (m, 2H), 4.25-4.09 (m, 1H), 3.87-3.74 (m, 1H), 3.63-3.47 (m, 2H), 3.44-3.34 (m, 8H), 2.98-2.82 (m, 1H), 2.81-2.53 (m, 4H), 2.42-2.31 (m, 2H), 2.25-2.14 (m, 2H), 2.01-1.91 (m, 1H), 1.82-1.56 (m, 3H), 1.26-1.04 (m, 2H).

### Example 43. Preparation of (1-308) 3-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione

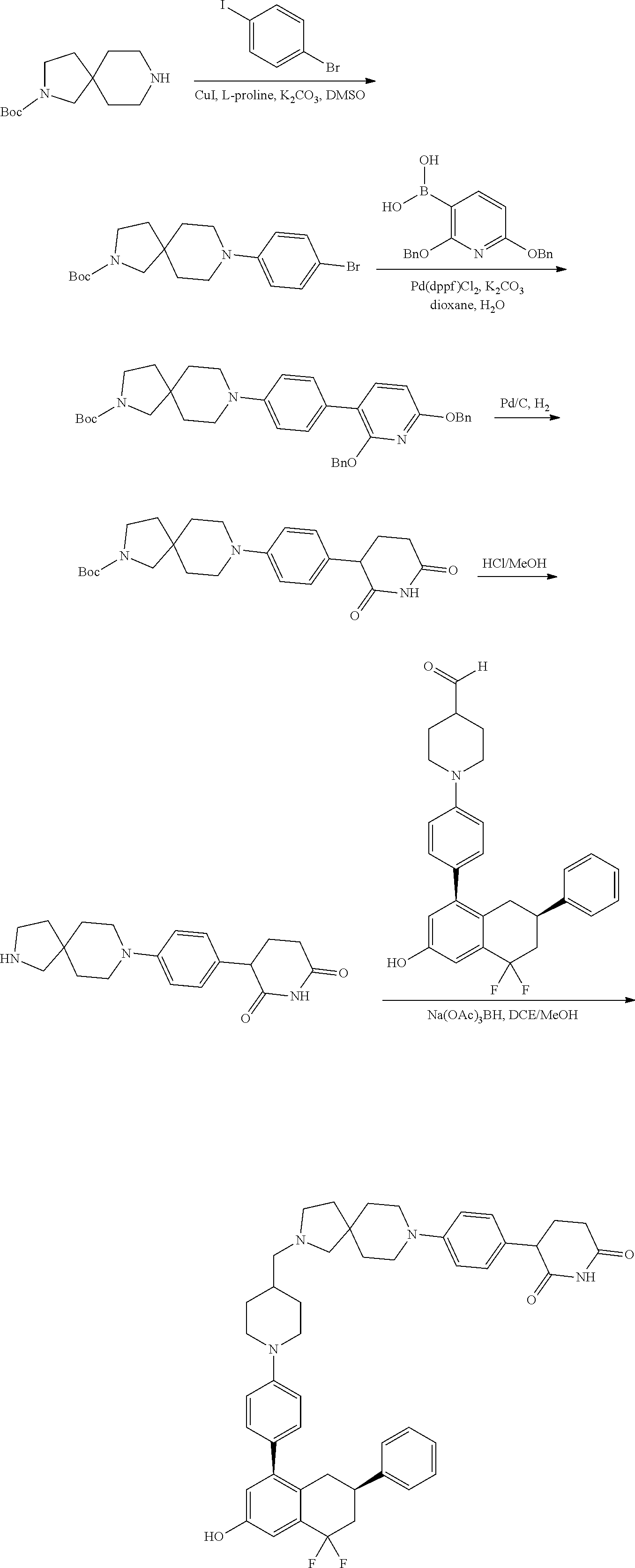

Step 1: A mixture of 1-bromo-4-iodo-benzene (600 mg, 2.12 mmol, 1 eq.), tert-butyl 2,8-diazaspiro[4.5]decane-2-carboxylate (611.68 mg, 2.55 mmol, 1.2 eq.), (2S)-pyrrolidine-2-carboxylic acid (146.50 mg, 1.27 mmol, 0.6 eq.), copper (I) iodide (242.35 mg, 1.27 mmol, 0.6 eq.) and K₂CO₃ (732.79 mg, 5.30 mmol, 2.5 eq.) in DMSO (3 mL) was degassed and purged with N₂ for 3 times, and then the mixture was stirred at 80° C. for 3 hour under N₂ atmosphere. LC-MS showed one main peak with desired mass was detected. The reaction mixture was partitioned between ethyl acetate (40 mL×2) and water (50 mL). After quenching the reaction, the reaction mixture was poured into separatory funnel and separated. The crude product was purified by silica gel chromatography eluted with PE:EtOAc=3:1. Tert-butyl 8-(4-bromophenyl)-2,8-diazaspiro[4.5]decane-2-carboxylate (260 mg, 657.67 μmol, 31.01% yield) was obtained as a white solid.

LC-MS (ESI⁺) m/z: 397.1 (M+H)⁺.

Step 2: A mixture of tert-butyl 8-(4-bromophenyl)-2,8-diazaspiro[4.5]decane-2-carboxylate (100 mg, 253 μmol, 1.0 eq.), (2,6-bis(benzyloxy)pyridin-3-yl) boronic acid (93.3 mg, 278 μmol, 1.1 eq.), K₂CO₃ (105 mg, 759 μmol, 3.0 eq.) and PdCl₂(dppf) (18.5 mg, 25.3 μmol, 0.1 eq.) in Dioxane (2 mL) and Water (0.5 mL) was degassed and purged with N₂ for 3 times, and then the mixture was stirred at 80° C. for 3 hour under N₂ atmosphere. LC-MS showed the reaction was completed. The reaction mixture was quenched by addition ethyl acetate (10 mL) at 20° C., and then diluted with water (10 mL) and extracted with ethyl acetate 30 mL (10 mL×3). The combined organic layers were dried over Na₂SO₄, filtered and concentrated under reduced pressure to give a residue. The crude material was purified on silica gel column chromatography (from 100% PE to PE/EtOAc=4/1) to give tert-butyl 8-(4-(2,6-bis(benzyloxy)pyridin-3-yl)phenyl)-2,8-diazaspiro[4.5]decane-2-carboxylate (120 mg, 0.16 mmol, 63% yield, 80% Purity) as a yellow solid.

LC-MS (ESI⁺) m/z: 606.3 (M+H)⁺.

Step 3: To a solution of tert-butyl 8-(4-(2,6-bis(benzyloxy)pyridin-3-yl)phenyl)-2,8-diazaspiro[4.5]decane-2-carboxylate (0.10 g, 0.17 mmol, 1.0 eq.) in EtOH (3 mL), DCM (3 mL) and EtOAc (3 mL) was added Pd/C (5 wt %, 0.1 g) under H₂ atmosphere. The suspension was degassed and purged 3× with H₂. The mixture was stirred under H₂ (30 psi) at 30° C. for 16 h. LC-MS showed the reaction was completed. The mixture was filtered and the filtrate was concentrated to give tert-butyl 8-(4-(2,6-dioxopiperidin-3-yl)phenyl)-2,8-diazaspiro[4.5]decane-2-carboxylate (65 mg, 0.15 mmol, 92%) as a white solid.

LC-MS (ESI⁺) m/z: 428.2 (M+H)⁺.

Step 4: To a solution of tert-butyl 8-(4-(2,6-dioxopiperidin-3-yl)phenyl)-2,8-diazaspiro[4.5]decane-2-carboxylate (60 mg, 0.14 mmol, 1.0 eq.) in HCl/MeOH (3 mL). The mixture was stirred at 20° C. for 1 hour. LCMS showed the reaction was completed. The mixture was concentrated in vacuum to give crude product. No further purification as it is used for the next step directly.

LC-MS (ESI+) m/z: 328.2 (M+H)⁺.

Step 5: To a solution of 1-(4-((1R,2S)-4,4-difluoro-6-hydroxy-2-phenyl-1,2,3,4-tetrahydronaphthalen-1-yl)phenyl)piperidine-4-carbaldehyde (35.0 mg, 78.2 μmol, 1.0 eq.) and 3-(4-(2,8-diazaspiro[4.5]decan-8-yl)phenyl)piperidine-2,6-dione (28.2 mg, 86.0 μmol, 1.1 eq.) in DCE (3 mL) and MeOH (3 mL) was added sodium triacetoxyborohydride (49.7 mg, 235 μmol, 3.8 eq.). The mixture was stirred at 20° C. for 2 h. LC-MS showed the reaction was complete. The crude was purified by prep-HPLC (Boston Green ODS 150*30 mm*5 um, water (FA)-ACN as a mobile phase, from 13% to 43%, Gradient Time (min): 12, Flow Rate (ml/min): 25) to give 3-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (16.9 mg, 22.3 μmol, 28.5% yield) as a white solid. LC-MS (ESI⁺) m/z: 759.5 (M+H)⁺.

LCMS: calc. for C₄₇H₅₂F₂N₄O₃:758.95, found: [M+H]⁺ 759.5.

HPLC: 100.00% purity at 220 nm.

¹H NMR (400 MHz, DMSO-d₄) δ: ppm 10.68-10.79 (m, 1H), 9.38-10.05 (m, 1H), 8.20 (s, 1H), 7.16-7.21 (m, 3H), 7.08 (s, 1H), 7.02 (d, J=8.58 Hz, 2H), 6.83-6.91 (m, 6H), 6.55 (d, J=8.70 Hz, 2H), 6.12 (d, J=8.58 Hz, 2H), 4.27-4.32 (m, 1H), 3.73 (s, 1H), 3.63-3.68 (m, 1H), 3.46-3.55 (m, 3H), 3.02-3.16 (m, 5H), 2.56-2.68 (m, 4H), 2.40-2.47 (m, 2H), 2.33 (s, 3H), 2.23 (d, J=7.15 Hz, 2H), 2.00-2.11 (m, 1H), 1.73 (d, J=10.97 Hz, 2H), 1.48-1.65 (m, 7H), 1.07-1.17 (m, 2H).

### Example 44. Preparation of (I-319) (S)-3-(5-(4-((1-(4-((1S,2R)-2-(2,4-difluorophenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione and (I-318) (S)-3-(5-(4-((1-(4-((1R,2S)-2-(2,4-difluorophenyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

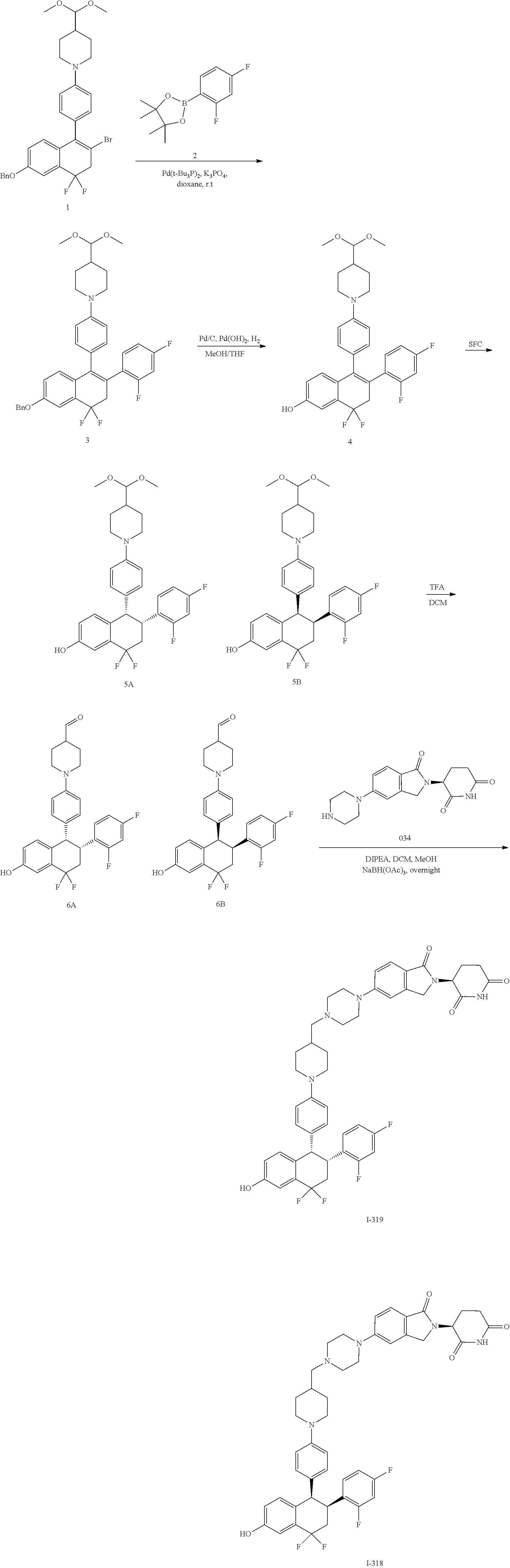

Step 1: To a mixture of compound 1 (500) mg, 855 mol, 1.0 eq.) and compound 2 (205 mg, 855 μmol, 1.0 eq.) in 1,4-Dioxane (8 mL) and water (2 mL) was added Pd(t-Bu₃P)₂ (87.4 mg, 0.2 eq., 171 μmol), K₃PO₄ (545 mg, 2.57 mmol, 3.0 eq.) in one portion at 25° C. under N₂. The mixture was stirred for 16 hours. The mixture was concentrated and then water (100 mL) was added. The mixture was extracted with EtOAc (80 mL×3). The combined organic layers were washed with brine (100 mL), dried over Na₂SO₄, filtered and concentrated. The crude material was purified on silica gel column chromatography (from PE/EtOAc=10/1 to 4/1) to yield compound 3 (437 mg, 707 μmol, 82.7% yield) as a yellow solid.

LC-MS (ESI⁺) m/z: 618.7 (M+H)⁺.

Step 2: To a solution of compound 3 (430 mg, 1 eq., 696 μmol) in THF (5 mL) and MeOH (5 mL) was added Pd(OH)₂ (196 mg, 10% Wt, 0.2 eq., 139 μmol) and Pd/C (148 mg, 10% Wt, 0.2 eq., 139 μmol). The suspension was degassed under vacuum and purged with H₂ several times. The mixture was heated to 45° C. (50 psi) and stirred for 16 hours. LCMS showed that the desired compound was detected. The crude product was filtered and the filter was concentrated to give the crude as a yellow solid. The mixture was filtered and concentrated in vacuum. The residue was purified by pre-HPLC(Column Boston Green ODS 150*30 mm*5 um; Condition water(FA)-ACN; Begin B 60; End B 90: Gradient Time (min) 12; 100% B Hold Time (min) 2; Flow Rate (ml/min) 25) to afford compound 4 (150 mg, 283 μmol, 40.7%) as a yellow solid.

LC-MS (ESI⁺) m/z: 530.5 (M+H)⁺.

¹H NMR (400 MHz, METHANOL-d₄) δ ppm: 7.13 (s, 1H), 6.84-6.98 (m, 3H), 6.62-6.70 (m, 3H), 6.38-6.46 (m, 1H), 6.32 (d, J=8.58 Hz, 2H), 4.31 (br d, J=3.34 Hz, 1H), 4.07 (d, J=6.79 Hz, 1H), 3.89-3.98 (m, 1H), 3.56 (br d, J=11.21 Hz, 2H), 3.35 (s, 6H), 3.14 (d, J=7.27 Hz, 4H), 2.49-2.57 (m, 2H), 2.25 (br t, J=14.96 Hz, 1H), 1.77 (br d, J=13.11 Hz, 2H).

Step 3: Compound 4 (150 mg, 283 μmol, 1.0 eq.) was separated by prep-SFC (Column DAICEL CHIRALPAK AD (250 mm*30 mm, 10 um); Condition CO₂-i-PrOH (0.1% NH₃H₂O); Begin B 25%; End B 25%: FlowRate (ml/min) 70) to give compound 5A (70 mg, 0.13 mmol, 47%) (Rt=3.908 min) and compound 5B (70 mg, 0.13 mmol, 47%) (Rt=4.098 min) as a white solid.

Step 4: Compound 6A: To a solution of compound 5A (50.00 mg, 94.42 μmol, 1.0 eq) in DCM (2 mL) was added TFA (10.77 mg, 94.42 μmol, 1.0 eq). The mixture was stirred at 25° C. for 0.2 hr. LCMS showed that the starting material was consumed and the desired product was detected. The reaction liquid is dried with N₂ at 0° C. to give Compound 6A (30 mg, 62.05 μmol, 65.72% yield) was obtained as yellow oil which was used in the next step without further purification.

LC-MS (ESI+) m/z: 484.21 (M+H)⁺.

Step 5: Compound 6B: To a solution of compound 5B (56.00 mg, 105.75 μmol, 1.0 eq) in DCM (2 mL) was added TFA (12.06 mg, 105.75 μmol, 1.0 eq). The mixture was stirred at 25° C. for 0.2 hr. LCMS showed that the starting material was consumed and the desired product was detected. The reaction liquid is dried with N₂ at 0° C. to give Compound 6B (32 mg, 66.18 μmol, 62.59% yield) was obtained as yellow oil which was used in the next step without further purification.

LC-MS (ESI+) m/z: 484.2 (M+H)⁺.

Step 6: To a solution of compound 6A (30 mg, 62.05 μmol, 1.0 eq) in DCM (3 mL) and MeOH (3 mL) was added compound 034 (20.37 mg, 62.05 μmol, 1.0 eq) and DIEA (8.02 mg, 62.05 μmol, 1.0 eq) at 25° C. After addition, the mixture was stirred at this temperature for 0.5 hr, and then NaBH(OAc)₃ (26.30 mg, 124.10 μmol, 2.0 eq) was added at 25° C. The resulting mixture was stirred at 25° C. for 3 hr. The reaction mixture was concentrated to yield a residue. The residue was purified by preparative HPLC (column: Boston Green ODS 150*30 mm*5 um; mobile phase: [water(NH₄HCO₃)-ACN]; B %: 52%-80%, 11 min), followed by lyophilization to yield I-319 (39.7 mg, 49.88 μmol, 80.39% yield) was obtained as a white solid. LC-MS (ESI+) m/z: 796.5 [M+H]⁺.

LCMS: calc. for C₄₅H₄₅F₄N₅O₄: 795.34, found: [M+H]⁺ 796.5.

HPLC: 100% purity at 220 nm.

¹H NMR (400 MHz, DMSO-d₆) δ ppm 10.94 (br s, 1H), 9.74-9.80 (m, 1H), 7.51 (d, J=8.58 Hz, 1H), 7.21-7.27 (m, 1H), 7.04-7.08 (m, 3H), 6.83-6.91 (m, 3H), 6.54-6.63 (m, 3H), 6.20 (d, J=8.46 Hz, 2H), 5.04 (dd, J=13.29, 5.07 Hz, 1H), 4.27-4.35 (m, 2H), 4.17-4.22 (m, 1H), 3.81 (br d, J=12.99 Hz, 1H), 3.55 (br s, 2H), 3.26-3.30 (m, 6H), 2.86-2.93 (m, 1H), 2.58-2.77 (m, 3H), 2.26-2.44 (m, 4H), 2.18 (br d, J=7.15 Hz, 2H), 1.93-1.98 (m, 1H), 1.75 (br d, J=12.28 Hz, 4H), 1.11-1.18 (m, 2H).

Step 7: To a solution of compound 6B (32 mg, 66.18 μmol, 1.0 eq) in DCM (3 mL) and MeOH (3 mL) was added compound 034 (21.73 mg, 66.18 μmol, 1.0 eq) and DIEA (8.55 mg, 66.18 μmol, 1.0 eq) at 25° C. After addition, the mixture was stirred at this temperature for 0.5 hr, and then NaBH(OAc)₃ (28.05 mg, 132.37 μmol, 2.0 eq) was added at 25° C. The resulting mixture was stirred at 25° C. for 3 hr. The reaction mixture was concentrated to yield a residue. The residue was purified by preparative HPLC (column: Boston Green ODS 150*30 mm*5 um; mobile phase: [water(NH₄HCO₃)-ACN]; B %: 52%-80%, 11 min), followed by lyophilization to yield I-318 (33.1 mg, 41.59 μmol, 62.84% yield) was obtained as a white solid. LC-MS (ESI+) m/z: 796.6 [M+H]⁺.

LCMS: calc. for C₄₅H₄₅F₄N₅O₄: 795.34, found: [M+H]⁺796.6.

HPLC: 100% purity at 220 nm

¹H NMR (400 MHZ, DMSO-d₆) δ ppm 10.95 (br s, 1H), 9.78 (s, 1H), 7.52 (d, J=8.70 Hz, 1H), 7.22-7.28 (m, 1H), 7.04-7.09 (m, 3H), 6.84-6.90 (m, 3H), 6.56-6.64 (m, 3H), 6.21 (d, J=8.70 Hz, 2H), 5.05 (dd, J=13.23, 5.01 Hz, 1H), 4.28-4.35 (m, 2H), 4.18-4.23 (m, 1H), 3.80-3.85 (m, 1H), 3.56 (br d, J=2.50 Hz, 2H), 3.28-3.31 (m, 6H), 2.87-2.94 (m, 1H), 2.60-2.76 (m, 3H), 2.28-2.45 (m, 4H), 2.19 (br d, J=7.03 Hz, 2H), 1.94-1.99 (m, 1H), 1.59-1.81 (m, 4H), 1.11-1.18 (m, 2H).

### Example 45. Preparation of (I-327) (S)-3-(5-(4-((1-(4-((1S,2R)-4,4-difluoro-2-(4-fluoro-2-methylphenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

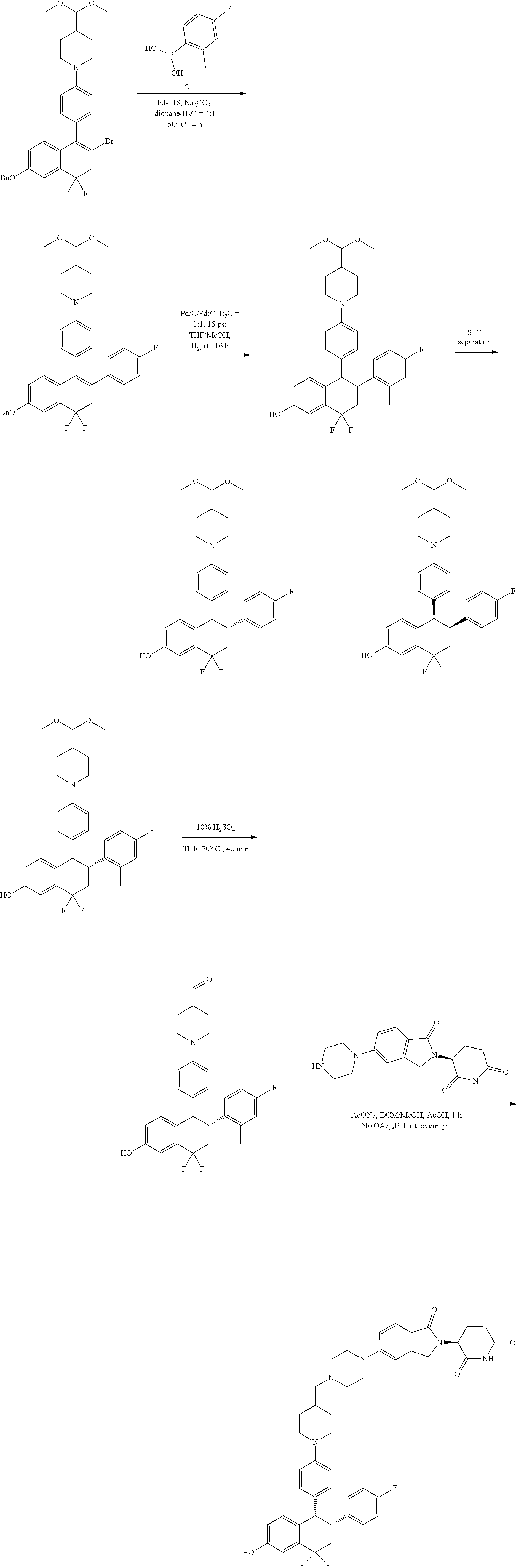

Step 1: A mixture of 1-(4-(6-(benzyloxy)-2-bromo-4,4-difluoro-3,4-dihydronaphthalen-1-yl)phenyl)-4-(dimethoxymethyl)piperidine (1.0 g, 1 Eq, 1.5 mmol), (4-fluoro-2-methylphenyl)boronic acid (0.28 g, 1.2 Eq, 1.8 mmol), Na₂CO₃ (0.48 g, 3 Eq, 4.5 mmol) and Pd-118 (98 mg, 0.1 Eq, 0.15 mmol) in dioxane (6 mL) and H₂O (1.5 mL) at r.t. N₂ was bubbled into the mixture for 5 min. The reaction mixture was heated at 50° C. for 4 h with N₂ The mixture was added with H₂O (100 mL) and extracted with EtOAc (100 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(6-(benzyloxy)-4,4-difluoro-2-(4-fluoro-2-methylphenyl)-3,4-dihydronaphthalen-1-yl)phenyl)-4-(dimethoxymethyl)piperidine (1 g, 90%, 80.3% purity) as a yellow solid. LC-MS (ESI+) m/z: 614.3 (M+H)⁺.

Step 2: A mixture of 1-(4-(6-(benzyloxy)-4,4-difluoro-2-(3-fluoro-5-methylphenyl)-3,4-dihydronaphthalen-1-yl)phenyl)-4-(dimethoxymethyl)piperidine (950 mg, 1 Eq, 1.24 mmol), Pd/C (500 mg, 10% Wt, 0.378 Eq, 470 μmol), Pd(OH) 2/C (500 mg, 20% Wt, 0.573 Eq, 712 μmol) in MeOH (7 mL) and THF (7 mL) was degassed and purged with H₂ for 3 times, then the mixture was stirred at 25° C. for 16 h with H₂ atmosphere (15 psi). The reaction mixture was filtered and concentrated under reduced pressure to give a residue. The residue was purified by HPLC (condition: column: Welch Xtimate C18 150*30 mm*5 um; mobile phase: [water (FA)-ACN]; B %: 36%-76%, 9 min), then was purified by SFC (condition: column: DAICEL CHIRALPAK AD (250 mm*30 mm, 10 um); mobile phase: [CO₂-EtOH]; B %: 25%-25%, min) to give (5R,6S)-5-(4-(4-(dimethoxymethyl)piperidin-1-yl)phenyl)-8,8-difluoro-6-(4-fluoro-2-methylphenyl)-5,6,7,8-tetrahydronaphthalen-2-ol (70 mg, 11%, 100% purity) and (5S,6R)-5-(4-(4-(dimethoxymethyl)piperidin-1-yl)phenyl)-8,8-difluoro-6-(4-fluoro-2-methylphenyl)-5,6,7,8-tetrahydronaphthalen-2-ol (40 mg, 6.1%, 100% purity) as white solids. LC-MS (ESI+) m/z: 526.1 (M+H)⁺.

Step 3: A mixture of (5S,6R)-5-(4-(4-(dimethoxymethyl)piperidin-1-yl)phenyl)-8,8-difluoro-6-(4-fluoro-2-methylphenyl)-5,6,7,8-tetrahydronaphthalen-2-ol (40 mg, 1 Eq, 76 μmol) in THF (1.5 mL), then 10% H₂SO₄ (1.5 mL) was added to the mixture and stirred at 70° C. for 0.5 h. The reaction was adjusted to pH=8 with aq. NaHCO₃ at 0° C. Then the mixture was added H₂O (10 mL) and extracted with EtOAc (20 mL×2). The combined organic layers were washed with brine (50 mL), dried over anhydrous Na₂SO₄, filtered and concentrated to dryness in vacuo to give 1-(4-((1S,2R)-4,4-difluoro-2-(4-fluoro-2-methylphenyl)-6-hydroxy-1,2,3,4-tetrahydronaphthalen-1-yl)phenyl)piperidine-4-carbaldehyde (37 mg, 99%, 97.206% purity) as a yellow solid. LC-MS (ESI+) m/z: 480.1 (M+H)⁺.

Step 4: A mixture of 1-(4-((1S,2R)-4,4-difluoro-2-(4-fluoro-2-methylphenyl)-6-hydroxy-1,2,3,4-tetrahydronaphthalen-1-yl)phenyl)piperidine-4-carbaldehyde (40 mg, 1 Eq, 81 μmol), (S)-3-(1-oxo-5-(piperazin-1-yl)isoindolin-2-yl)piperidine-2,6-dione benzenesulfonate (51 mg, 1.3 Eq, 0.11 mmol), sodium acetate (33 mg, 5 Eq, 0.41 mmol) and acetic acid (15 mg, 14 μL, 3 Eq, 0.24 mmol) in DCM (3 mL) and MeOH (3 mL) was stirred at 25° C. for 1 h, then NaBH(OAc); (34 mg, 2 Eq, 0.16 mmol) was added to the mixture and was stirred at 25° C. for 16 h. Then the mixture was added H₂O (20 mL) and extracted with EtOAc (20 mL×2). The combined organic layers were washed with brine (20 mL), dried over anhydrous Na₂SO₄, filtered and concentrated to dryness in vacuo to give a residue. The residue was purified by prep-HPLC (column: Welch Xtimate C18 150*30 mm*5 um; mobile phase: [water (FA)-ACN]; B % 15%-55%, 9 min) to give (S)-3-(5-(4-((1-(4-((1S,2R)-4,4-difluoro-2-(4-fluoro-2-methylphenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (21.8 mg, 34%, 100% purity) as a white solid. LC-MS (ESI+) m/z: 792.4 (M+H)⁺.

LCMS: calc. for C₄₆H₄₈F₃N₅O₄: 791.37, found: [M+H]⁺ 792.4.

HPLC: 100% purity at 220 nm.

¹HNMR (400 MHz, DMSO-d₆) δ=10.96 (s, 1H), 9.76 (br s, 1H), 8.15 (s, 1H), 7.52 (d, J=8.5 Hz, 1H), 7.12-7.01 (m, 4H), 6.94-6.83 (m, 2H), 6.73-6.55 (m, 3H), 6.21-6.08 (m, 3H), 5.05 (dd, J=5.0, 13.2 Hz, 1H), 4.37-4.28 (m, 2H), 4.24-4.17 (m, 1H), 3.71 (br d, J=12.6 Hz, 1H), 3.55 (br d, J=9.4 Hz, 2H), 3.28 (br s, 5H), 2.97-2.84 (m, 1H), 2.79-2.53 (m, 4H), 2.47 (s, 6H), 2.37 (dq, J=4.9, 13.3 Hz, 1H), 2.27-2.15 (m, 3H), 2.00-1.91 (m, 1H), 1.82-1.59 (m, 3H), 1.23-1.09 (m, 2H).

### Example 46. Preparation of (I-305) 3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

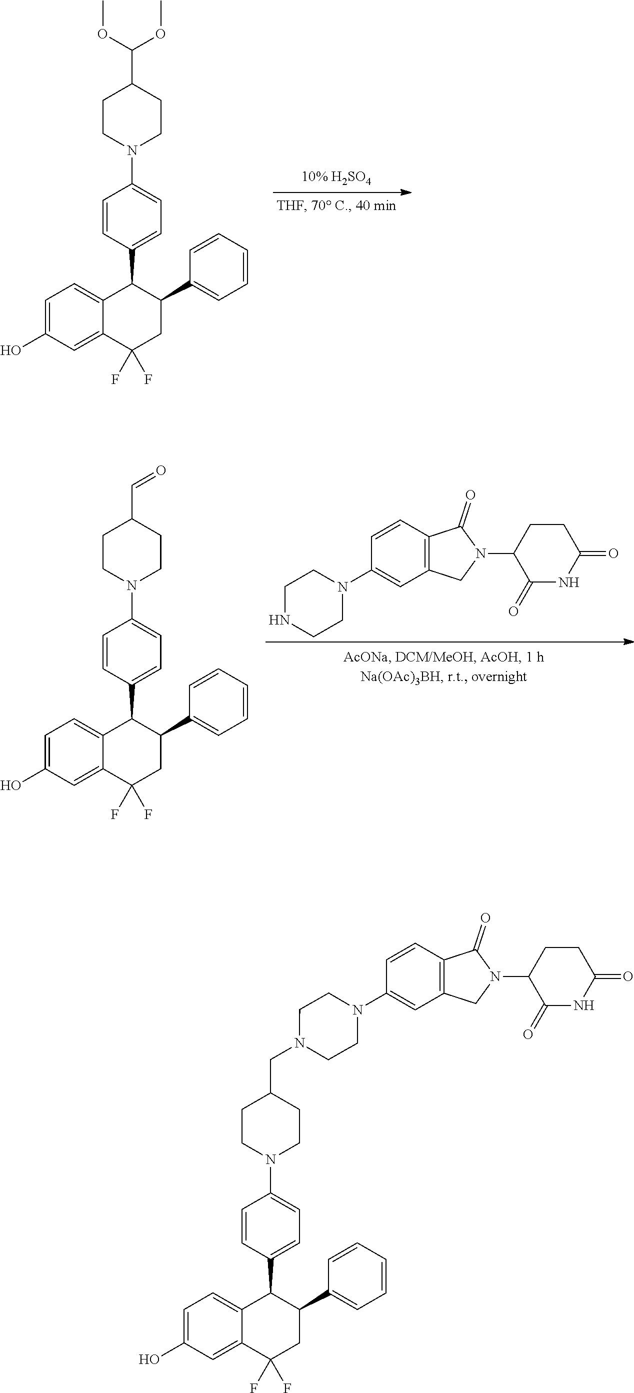 [SMILES: COC(OC)C1CCN(c2ccc([C@@H]3c4ccc(O)cc4C(F)(F)C[C@@H]3c3ccccc3)cc2)CC1 ; O=CC1CCN(c2ccc([C@@H]3c4ccc(O)cc4C(F)(F)C[C@@H]3c3ccccc3)cc2)CC1 ; O=C1CCC(N2Cc3cc(N4CCNCC4)ccc3C2=O)C(=O)N1 ; O=C1CCC(N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1]

Step 1: To a solution of (5S,6R)-5-(4-(4-(dimethoxymethyl)piperidin-1-yl)phenyl)-8,8-difluoro-6-phenyl-5,6,7,8-tetrahydronaphthalen-2-ol (60 mg, 1 Eq, 0.12 mmol) in THF (S mL) was added sulfuric acid (0.12 g, 5 mL, 10% Wt % in water, 1.0 Eq, 0.12 mmol). The mixture was stirred at 70° C. for 30 min. The mixture was adjusted to pH=8-9 by progressively adding saturated aqueous NaHCO₃. The reaction mixture was quenched by addition 100 mL EtOAc at 20° C., and then diluted with 50 mL H₂O and extracted with EtOAc (25 mL*2). The combined organic layers were washed with brine (10 mL), dried over anhydrous Na₂SO₄. The reaction was filtered and concentrated under reduced pressure to give 1-(4-((1S,2R)-4,4-difluoro-6-hydroxy-2-phenyl-1,2,3,4-tetrahydronaphthalen-1-yl)phenyl)piperidine-4-carbaldehyde (42 mg, 80 μmol, 66% yield, 84.8% purity) as a colorless oil. LC-MS (ESI+) m/z: 466.1 (M+H)⁺.

Step 2: A mixture of 1-(4-((1S,2R)-4,4-difluoro-6-hydroxy-2-phenyl-1,2,3,4-tetrahydronaphthalen-1-yl)phenyl)piperidine-4-carbaldehyde (42.0 mg, 1 Eq, 79.6 μmol), 3-(1-oxo-5-(piperazin-1-yl)isoindolin-2-yl)piperidine-2,6-dione (26.1 mg, 1 Eq, 79.6 μmol, HCl salt) and sodium acetate (32.7 mg, 5 Eq, 398 μmol) in DCM (4 mL) and MeOH (4 mL) at 20° C. for was stirred for 1 h. Acetic acid (14.3 mg, 13.7 μL, 3 Eq, 239 μmol) was added at 20° C. and stirred for an additional 1 h, then added Na(OAc)₃BH (33.7 mg, 2 Eq, 159 μmol) was added to the mixture and stirred at 25° C. for 16 h. The reaction was quenched with water (50 mL) and extracted with ethyl acetate (50 mL*2). The reaction was concentrated to give 3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione as a brown oil. The brown oil was purified by preparative high-performance liquid chromatography. Condition: water (FA)-ACN. Column: Welch Xtimate C18 150*30 mm*5 um. Begin B: 21%, End B: 51%. Gradient Time (min) 7; 100% B hold Time (min) 2.7, Flow Rate (ml/min) 25. The pure fractions were collected, and the solvent was evaporated under vacuum. The aqueous layer was lyophilized to dryness to give 3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (27 mg, 35 μmol, 44% yield, 99.2% purity) as a white solid.

LCMS: calc. for C₄₅H₄₇F₂N₅O₄: 759.36, found: [M+H]⁺ 760.3.

HPLC: 99.22% purity at 220 nm.

¹H NMR (400 MHZ, DMSO-d₆) δ=10.99-10.92 (m, 1H), 9.79-9.69 (m, 1H), 8.16-8.14 (m, 0.443H), 7.54-7.49 (m, 1H), 7.22-7.17 (m, 3H), 7.06 (s, 3H), 6.92-6.89 (m, 2H), 6.86 (br d, J=4.4 Hz, 2H), 6.60-6.53 (m, 2H), 6.16-6.08 (m, 2H), 5.08-5.00 (m, 1H), 4.36-4.27 (m, 2H), 4.23-4.15 (m, 1H), 3.70-3.62 (m, 1H), 3.57-3.49 (m, 2H), 3.27 (br s, 5H), 2.95-2.84 (m, 1H), 2.71-2.52 (m, 4H), 2.48-2.45 (m, 3H), 2.38-2.27 (m, 2H), 2.21-2.15 (m, 2H), 1.99-1.91 (m, 1H), 1.78-1.70 (m, 2H), 1.67-1.58 (m, 1H), 1.20-1.08 (m, 2H).

### Example 47. Preparation of (I-306) (R)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

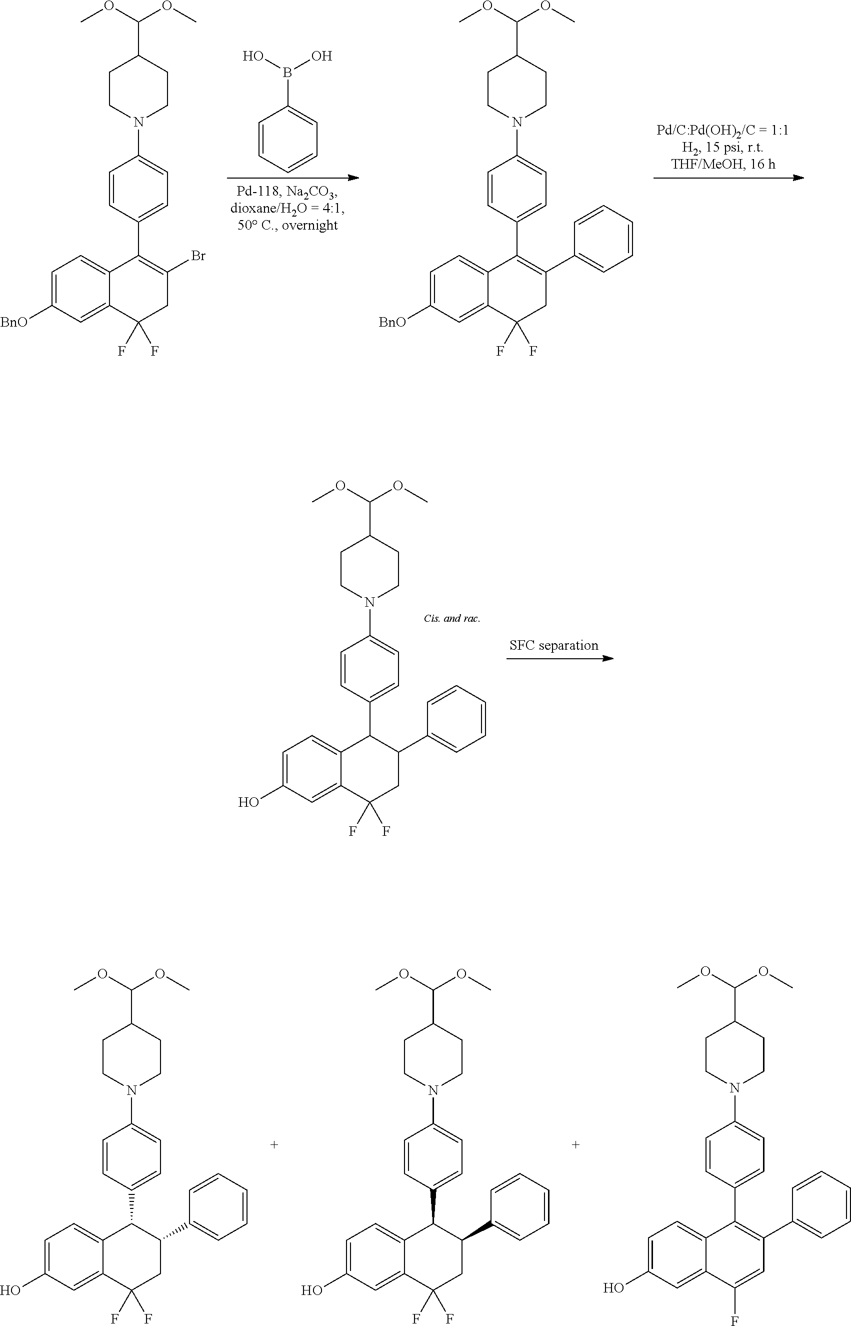

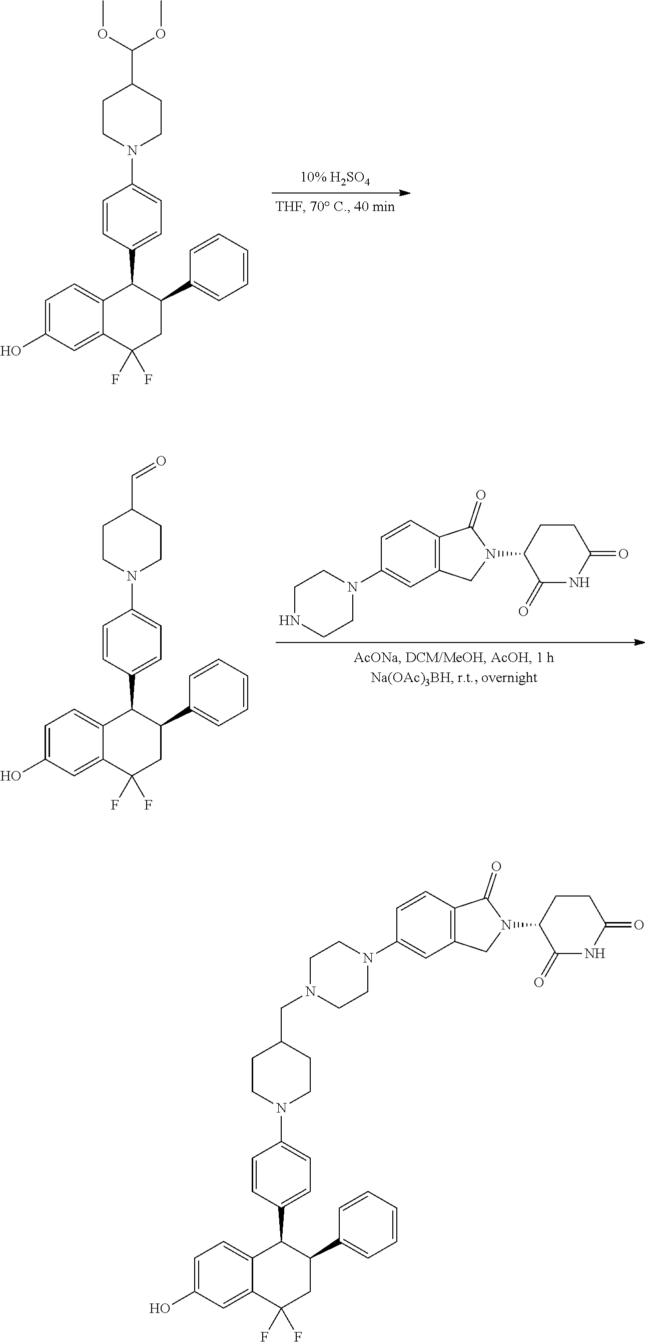 [SMILES: COC(OC)C1CCN(c2ccc([C@@H]3c4ccc(O)cc4C(F)(F)C[C@@H]3c3ccccc3)cc2)CC1 ; O=CC1CCN(c2ccc([C@@H]3c4ccc(O)cc4C(F)(F)C[C@@H]3c3ccccc3)cc2)CC1 ; O=C1CC[C@@H](N2Cc3cc(N4CCNCC4)ccc3C2=O)C(=O)N1 ; O=C1CC[C@@H](N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1]

Step 1: 1,1′-Bis(di-t-butylphosphino)ferrocene palladium dichloride (97.9 mg, 0.1 Eq, 150 μmol) was added to the mixture of 1-(4-(6-(benzyloxy)-2-bromo-4,4-difluoro-3,4-dihydronaphthalen-1-yl)phenyl)-4-(dimethoxymethyl)piperidine (1 g, 1 Eq, 1.50 mmol), phenylboronic acid (275 mg, 1.5 Eq, 2.25 mmol) and sodium carbonate (478 mg, 3 Eq, 4.51 mmol) in dioxane (20 mL) and H₂O (5 mL) at r.t. N₂ was bubbled into the mixture for 5 min. The reaction mixture was heated at 50° C. for 16 hour. TLC (PE/EtOAc=5/1, R_{f}=0.3) showed a new spot was detected. The mixture was filtered and the filter cake was washed with EtOAc (150 mL). The filtrate was treated with H₂O (150 mL), extracted with EtOAc (150 mL*2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 1-(4-(6-(benzyloxy)-4,4-difluoro-2-phenyl-3,4-dihydronaphthalen-1-yl)phenyl)-4-(dimethoxymethyl)piperidine (1.1 g, 1.7 mmol, 88.1% purity) as a brown solid. LC-MS (ESI⁺) m/z: 582.1 (M+H)⁺.

Step 2: A mixture of 1-(4-(6-(benzyloxy)-4,4-difluoro-2-phenyl-3,4-dihydronaphthalen-1-yl)phenyl)-4-(dimethoxymethyl)piperidine (1.1 g, 1 Eq, 1.7 mmol), dihydrogen (3.4 mg, 1 Eq, 1.7 mmol), Pd/C (550 mg, 10% Wt) and Pd(OH) 2/C (550 mg, 20% Wt) in THF (30 mL) and MeOH (30 mL) was degassed and purged with H₂ for 3 times, and then the mixture was stirred at 25° C. for 16 hour under 12 atmosphere (15 psi). The reaction was filtered and concentrated to dryness in vacuo to give 5-(4-(4-(dimethoxymethyl)piperidin-1-yl)phenyl)-8,8-difluoro-6-phenyl-5,6,7,8-tetrahydronaphthalen-2-ol (839 mg, 1.52 mmol, 91%, 89.50% purity) as a brown solid. LC-MS (ESI⁺) m/z: 494.3 (M+H)⁺.

Step 3: The brown solid was purified by SFC. Column: DAICEL CHIRALPAK AD (250 mm*30 mm, 10 um); Condition: CO₂-EtOH (0.1% NH₃H₂O); At the beginning: B (35%); At the end: B (35%); Flow Rate (ml/min) 80. The aqueous phase was lyophilized to dryness to give (5*S,6*R)-5-(4-(4-(dimethoxymethyl)piperidin-1-yl)phenyl)-8,8-difluoro-6-phenyl-5,6,7,8-tetrahydronaphthalen-2-ol (341 mg, 688 μmol, 45.2%, 99.58% purity) as a colorless oil, (5*R,6*S)-5-(4-(4-(dimethoxymethyl)piperidin-1-yl)phenyl)-8,8-difluoro-6-phenyl-5,6,7,8-tetrahydronaphthalen-2-ol (350 mg, 684 μmol, 45.0%, 96.44% purity) as a colorless oil and 5-(4-(4-(dimethoxymethyl)piperidin-1-yl)phenyl)-8-fluoro-6-phenylnaphthalen-2-ol (52 mg, 0.10 mmol, 6.7%, 93% purity) as a white solid. LC-MS (ESI⁺) m/z: 494.1 and 472.1 (M+H)⁺

Step 4: To a solution of (5*S,6*R)-5-(4-(4-(dimethoxymethyl)piperidin-1-yl)phenyl)-8,8-difluoro-6-phenyl-5,6,7,8-tetrahydronaphthalen-2-ol (90 mg, 1 Eq, 0.18 mmol) in THF (5 mL) was added 10% sulfuric acid (0.17 g, 5 mL). The mixture was stirred at 70° C. for 30 min. The mixture was adjusted to pH 8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL (10 mL*1), dried over Na₂SO₄. The reaction was filtered and concentrated under reduced pressure to give 1-(4-((1*S,2*R)-4,4-difluoro-6-hydroxy-2-phenyl-1,2,3,4-tetrahydronaphthalen-1-yl)phenyl)piperidine-4-carbaldehyde (100 mg, 222 μmol, 99.5% purity) as a colorless oil. LC-MS (ESI⁺) m/z: 466.0 (M+H)⁺.

Step 5: A mixture of 1-(4-((1R,2S)-4,4-difluoro-6-hydroxy-2-phenyl-1,2,3,4-tetrahydronaphthalen-1-yl)phenyl)piperidine-4-carbaldehyde (100 mg, 1 Eq, 222 μmol), (R)-3-(1-oxo-5-(piperazin-1-yl)isoindolin-2-yl)piperidine-2,6-dione hydrochloride (81.1 mg, 1 Eq, 222 μmol) and sodium acetate (91.2 mg, 5 Eq, 1.11 mmol) in DCM (4 mL) and MeOH (4 mL) at 20° C. for 0.5 hour, then acetic acid is added (40.1 mg, 38.1 μL, 3 Eq, 667 μmol) at 20° C. for 1 h, then added sodium triacetoxyborohydride (94.2 mg, 2 Eq, 445 μmol) to the mixture, and then the mixture was stirred at 25° C. for 16 h. The reaction was quenched with water (50 mL) and extracted with ethyl acetate (50 mL*2). The reaction was concentrated to give (R)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione as a brown oil. The brown oil was purified by preparative high-performance liquid chromatography. Condition: water (FA)-ACN. Column: Welch Xtimate C18 150*30 mm*5 um. Begin B: 20%, End B: 60%. Gradient Time (min) 9; 100% B hold Time (min) 3, Flow Rate (ml/min) 30. The pure fractions were collected, and the solvent was evaporated under vacuum. The aqueous layer was lyophilized to dryness to give (R)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (57.9 mg, 76.2 μmol, 34.3%, 100% purity) as a white solid.

LCMS: calc. for C₄₅H₄₇F₂N₅O₄: 759.36, found: [M+H]⁺ 760.4.

HPLC: 100% purity at 220 nm.

¹H NMR (400 MHZ, DMSO-d₆) δ=10.98-10.90 (m, 1H), 9.78-9.67 (m, 1H), 8.14-8.12 (m, 0.124H), 7.55-7.48 (m, 1H), 7.23-7.16 (m, 3H), 7.11-7.03 (m, 3H), 6.93-6.83 (m, 4H), 6.61-6.52 (m, 2H), 6.18-6.09 (m, 2H), 5.09-5.00 (m, 1H), 4.37-4.27 (m, 2H), 4.24-4.15 (m, 1H), 3.70-3.61 (m, 1H), 3.57-3.49 (m, 2H), 3.31-3.18 (m, 6H), 2.95-2.84 (m, 1H), 2.72-2.52 (m, 5H), 2.49-2.46 (m, 1H), 2.39-2.17 (m, 4H), 2.00-1.91 (m, 1H), 1.79-1.71 (m, 2H), 1.70-1.59 (m, 1H), 1.21-1.08 (m, 2H).

### Example 48. Preparation of (I-309) 3-(5-(4-((1-(4-((1R,2S)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

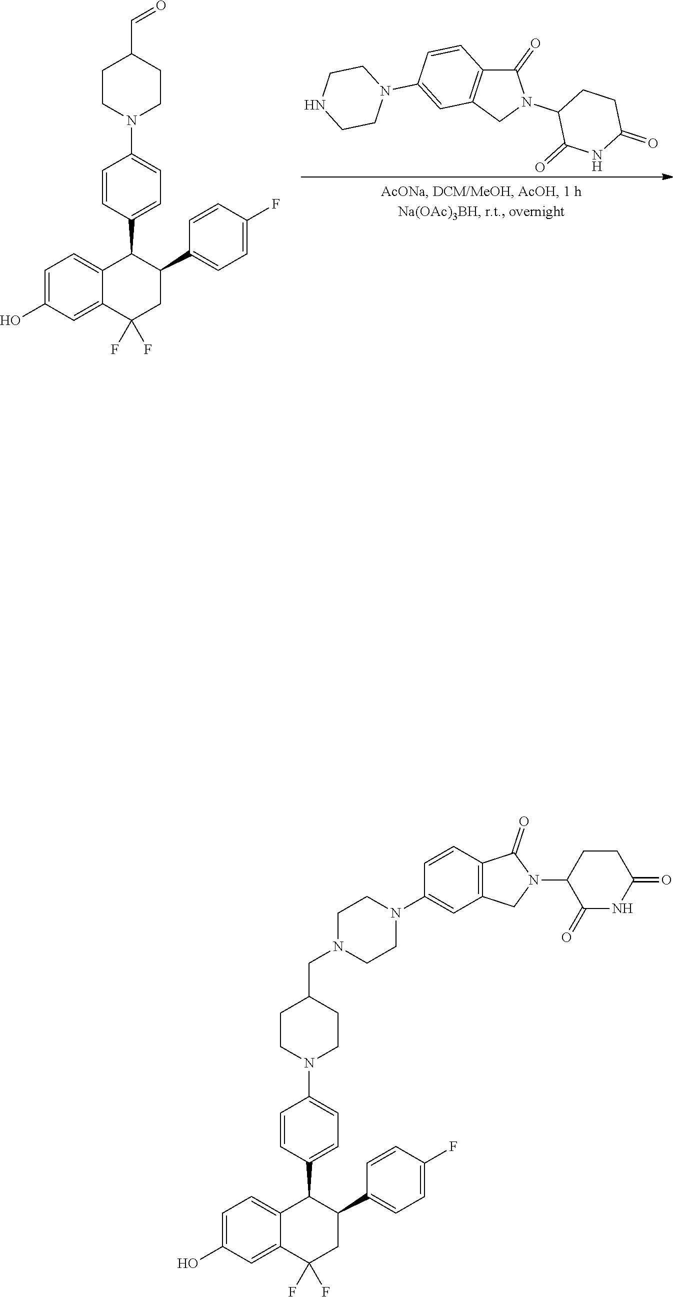 [SMILES: O=CC1CCN(c2ccc([C@@H]3c4ccc(O)cc4C(F)(F)C[C@@H]3c3ccc(F)cc3)cc2)CC1 ; O=C1CCC(N2Cc3cc(N4CCNCC4)ccc3C2=O)C(=O)N1 ; O=C1CCC(N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1]

Step 1: A mixture of 1-(4-((1R,2S)-4,4-difluoro-2-(4-fluorophenyl)-6-hydroxy-1,2,3,4-tetrahydronaphthalen-1-yl)phenyl)piperidine-4-carbaldehyde (80 mg, 1.0 Eq, 0.17 mmol), 3-(1-oxo-5-(piperazin-1-yl)isoindolin-2-yl)piperidine-2,6-dione, HCl salt (80 mg, 1.3 Eq, 0.22 mmol) and sodium acetate (69 mg, 5 Eq, 0.84 mmol) and acetic acid (30 mg, 29 μL, 3 Eq, 0.50 mmol) diluted in THF (3 mL) and MeOH (3 mL) at 25° C. and stirred for 1 h. Then sodium triacetoxyborohydride (71 mg, 50 μL, 2 Eq, 0.34 mmol) was added to the mixture and was stirred at 25° C. for 16 h. The mixture was added H₂O (20 mL) and extracted with EtOAc (20 mL×2). The combined extracts were dried over anhydrous Na₂SO₄, filtered and concentrated to dryness in vacuo to give a residue. The residue was purified by prep-HPLC (column: Welch Xtimate C18 150*30 mm*5 um; mobile phase: [water (FA)-ACN]; B % 15%-55%, 9 min) to give 3-(5-(4-((1-(4-((1R,2S)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (61 mg, 46%, 99.262% purity) as a white solid. LC-MS (ESI+) m/z: 778.4 (M+H)⁺.

LCMS: calc. for C₄₅H₄₆F₃N₅O₄: 777.35, found: [M+H]⁺ 778.4.

HPLC: 99.262% purity at 220 nm.

¹HNMR (400 MHz, DMSO-d₆) δ=10.95 (s, 1H), 9.74 (s, 1H), 8.14 (s, 1H), 7.53 (br d, J=8.1 Hz, 1H), 7.10-7.00 (m, 5H), 6.97-6.91 (m, 2H), 6.90-6.83 (m, 2H), 6.61 (br d, J=8.5 Hz, 2H), 6.15 (d, J=8.5 Hz, 2H), 5.05 (dd, J=4.9, 13.4 Hz, 1H), 4.36-4.18 (m, 3H), 3.70 (br d, J=11.2 Hz, 1H), 3.56 (br s, 2H), 3.28 (br s, 3H), 2.96-2.85 (m, 1H), 2.73-2.53 (m, 7H), 2.49-2.15 (m, 6H), 2.02-1.91 (m, 1H), 1.82-1.60 (m, 3H), 1.16 (br d, J=9.8 Hz, 2H).

### Example 49. Preparation of (I-310) (R)-3-(5-(4-((1-(4-((1R,2S)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

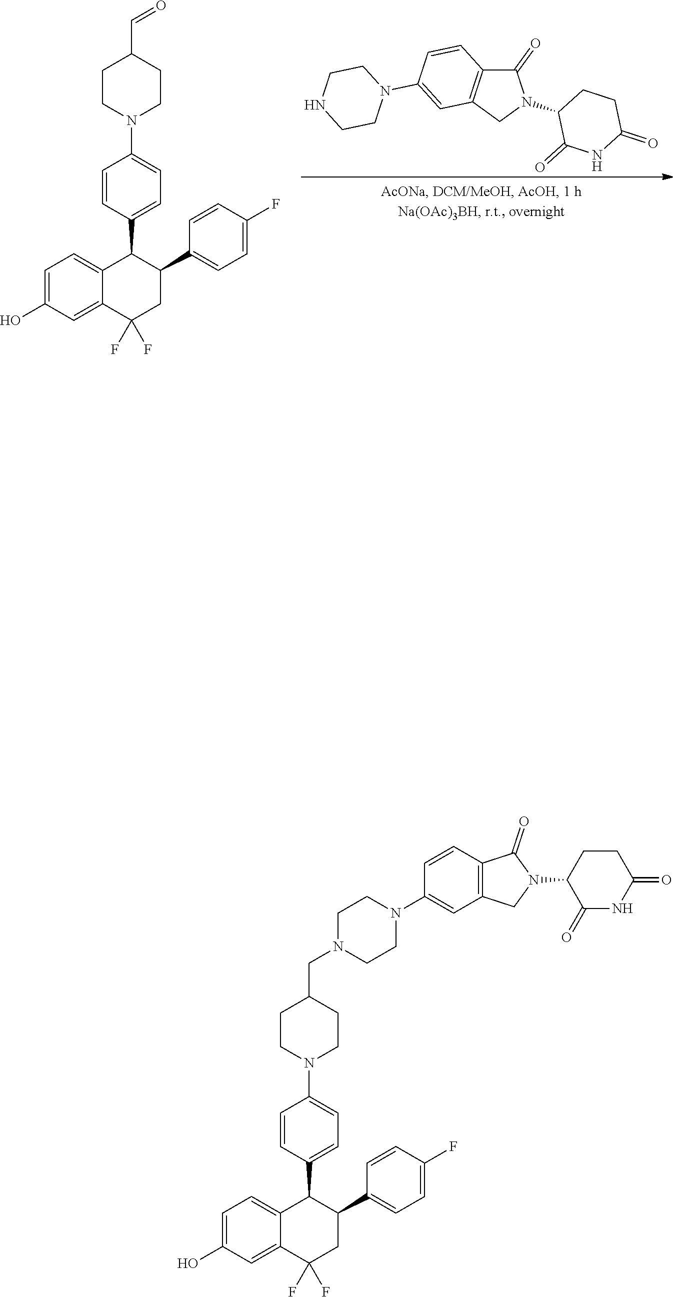 [SMILES: O=CC1CCN(c2ccc([C@@H]3c4ccc(O)cc4C(F)(F)C[C@@H]3c3ccc(F)cc3)cc2)CC1 ; O=C1CC[C@@H](N2Cc3cc(N4CCNCC4)ccc3C2=O)C(=O)N1 ; O=C1CC[C@@H](N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1]

Step 1: A mixture of 1-(4-((1R,2S)-4,4-difluoro-2-(4-fluorophenyl)-6-hydroxy-1,2,3,4-tetrahydronaphthalen-1-yl)phenyl)piperidine-4-carbaldehyde (50 mg, 1 Eq, 0.11 mmol), (R)-3-(1-oxo-5-(piperazin-1-yl)isoindolin-2-yl)piperidine-2,6-dione, HCl salt (50 mg, 1.3 Eq, 0.14 mmol) and sodium acetate (43 mg, 5 Eq, 0.53 mmol) and acetic acid (19 mg, 18 μL, 3 Eq, 0.32 mmol) were diluted in THF (3 mL) and MeOH (3 mL) at 25° C. for 1 h, then sodium triacetoxyborohydride (45 mg, 2 Eq, 0.21 mmol) was added to the mixture and was stirred at 25° C. for 16 h. The mixture was added to H₂O (20 mL) and extracted with EtOAc (20 mL×2). The combined extracts were dried over anhydrous Na₂SO₄, filtered and concentrated to dryness in vacuo to give a residue. The residue was purified by prep-HPLC (column: Welch Xtimate C18 150*30 mm*5 um; mobile phase: [water (FA)-ACN]; B % 15%-55%, 9 min) to give (R)-3-(5-(4-((1-(4-((1R,2S)-4,4-difluoro-2-(4-fluorophenyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (21.6 mg, 26%, 100% purity) as a white solid. LC-MS (ESI+) m/z: 778.4 (M+H)⁺.

LCMS: calc. for C₄₅H₄₆F₃N₅O₄: 777.35, found: [M+H]⁺ 778.4.

HPLC: 100% purity at 220 nm.

¹HNMR (400 MHz, DMSO-d₆) δ=10.95 (s, 1H), 9.74 (s, 1H), 8.14 (s, 1H), 7.53 (d, J=8.5 Hz, 1H), 7.10-7.00 (m, 5H), 6.97-6.91 (m, 2H), 6.90-6.83 (m, 2H), 6.61 (d, J=8.6 Hz, 2H), 6.15 (d, J=8.6 Hz, 2H), 5.05 (dd, J=5.1, 13.2 Hz, 1H), 4.36-4.27 (m, 2H), 4.24-4.18 (m, 1H), 3.70 (br d, J=13.2 Hz, 1H), 3.55 (br s, 2H), 3.30 (br s, 4H), 2.97-2.84 (m, 1H), 2.73-2.52 (m, 8H), 2.50-2.49 (m, 1H), 2.44-2.18 (m, 3H), 2.01-1.92 (m, 1H), 1.80-1.62 (m, 3H), 1.24-1.09 (m, 2H).

### Example 50. Preparation of (I-292) 1-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)dihydropyrimidine-2,4 (1H,3H)-dione

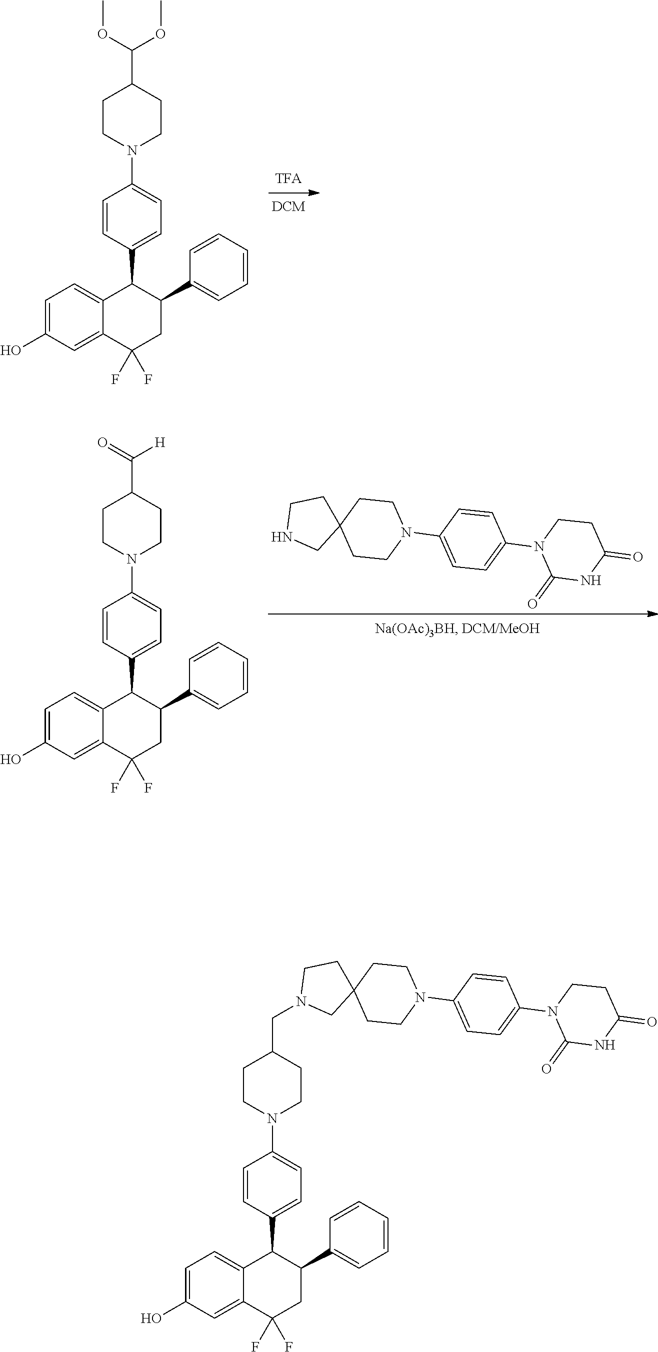 [SMILES: COC(OC)C1CCN(c2ccc([C@@H]3c4ccc(O)cc4C(F)(F)C[C@@H]3c3ccccc3)cc2)CC1 ; O=CC1CCN(c2ccc([C@@H]3c4ccc(O)cc4C(F)(F)C[C@@H]3c3ccccc3)cc2)CC1 ; O=C1CCN(c2ccc(N3CCC4(CCNC4)CC3)cc2)C(=O)N1 ; O=C1CCN(c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1]

Step 1: To a solution of (5R,6S)-5-(4-(4-(dimethoxymethyl)piperidin-1-yl)phenyl)-8,8-difluoro-6-phenyl-5,6,7,8-tetrahydronaphthalen-2-ol (50.0 mg, 101 μmol, 1.0 eq.) in DCM (3 mL) was added TFA (173 mg, 117 μL, 15 eq., 1.52 mmol). The mixture was stirred at 20° C. for 30 min. LCMS showed the reaction was completed. The mixture was concentrated in vacuum to give crude product. No further purification as it is used for the next step directly.

LC-MS (ESI⁺) m/z: 448.3 (M+H)⁺.

Step 2: To a solution of 1-(4-((1R,2S)-4,4-difluoro-6-hydroxy-2-phenyl-1,2,3,4-tetrahydronaphthalen-1-yl)phenyl)piperidine-4-carbaldehyde (35.0 mg, 78.2 μmol, 1.0 eq.) and 1-(4-(2,8-diazaspiro[4.5]decan-8-yl)phenyl)dihydropyrimidine-2,4 (1H,3H)-dione (28.3 mg, 86.0 μmol, 1.1 eq.) in DCE (3 mL) and MeOH (1 mL) was added Sodium triacetoxyborohydride (49.7 mg, 235 μmol, 3.0 eq.). The mixture was stirred at 20° C. for 2 h. LC-MS showed the reaction was completed. The crude was purified by prep-HPLC together (Boston Green ODS 150*30 mm*5 um, water (FA)-ACN as a mobile phase, from 13% to 43%, Gradient Time (min): 14, Flow Rate (ml/min): 25) to give 1-(4-(2-((1-(4-((1R,2S)-4,4-difluoro-6-hydroxy-2-phenyl-1,2,3,4-tetrahydronaphthalen-1-yl)phenyl)piperidin-4-yl)methyl)-2,8-diazaspiro[4.5]decan-8-yl)phenyl)dihydropyrimidine-2,4 (1H,3H)-dione (14.9 mg, 19.6 μmol, 25.1% yield) as a white solid. LC-MS (ESI⁺) m/z: 760.4 (M+H)⁺.

LCMS: calc. for C₄₆H₅₁F₂N₅O₃: 759.94, found: [M+H]⁺ 760.4.

HPLC: 100.00% purity at 220 nm.

¹H NMR (400 MHZ, DMSO-d₄) δ: ppm 10.25 (s, 1H), 9.66-9.77 (m, 1H), 8.16 (s, 1H), 7.15-7.23 (m, 3H), 7.12 (d, J=8.80 Hz, 2H), 7.08 (s, 1H), 6.83-6.94 (m, 6H), 6.55 (d, J=8.40 Hz, 2H), 6.12 (d, J=8.40 Hz, 2H), 4.79-4.79 (m, 1H), 4.30 (d, J=2.40 Hz, 1H), 3.63-3.71 (m, 3H), 3.47-3.55 (m, 2H), 3.27-3.31 (m, 4H), 3.04-3.17 (m, 4H), 2.67 (t, J=6.40 Hz, 3H), 2.34 (d, J=14.40 Hz, 3H), 2.25 (d, J=7.20 Hz, 2H), 1.73 (d, J=12.80 Hz, 2H), 1.45-1.67 (m, 7H), 1.05-1.19 (m, 2H).

### Example 51. Preparation of (I-281) 3-(4-((S)-7-((1-(4-((1R,2S)-4,4-difluoro-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)piperidine-2,6-dione

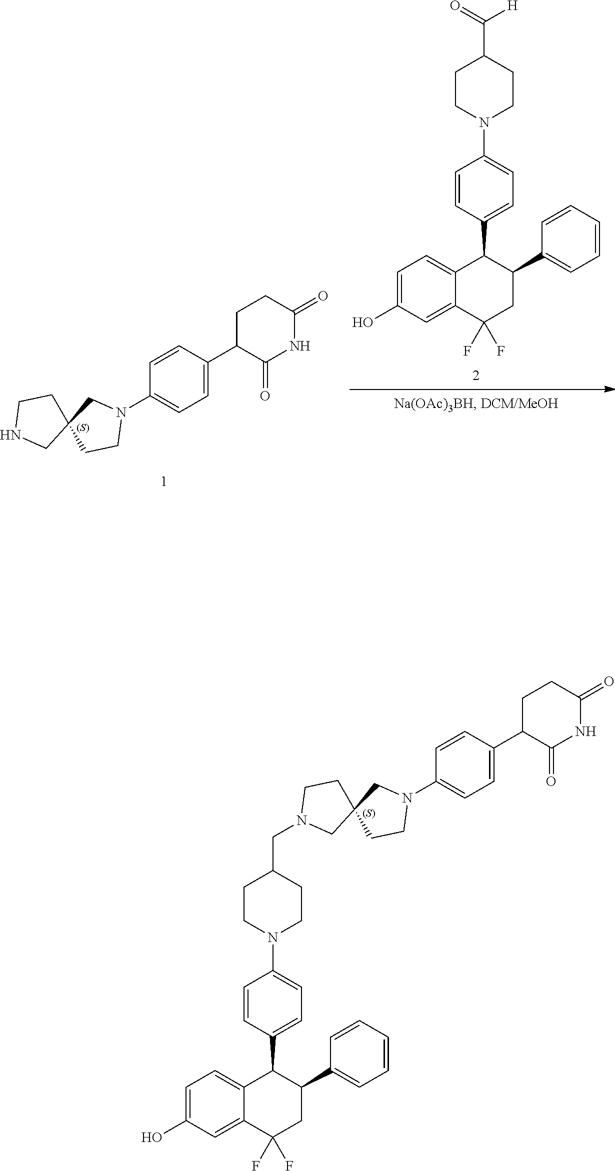

Step 1: To a solution of compound 1 (20.00 mg, 57.17 μmol, 1.0 eq, HCl) in MeOH (1 mL) and DCM (2 ml) was added compound 2 (12.79 mg, 28.58 μmol, 0.5 eq.) at 25° C. After addition, the mixture was stirred at this temperature for 0.5 hr, and then NaBH₃(OAc)₃ (12.12 mg, 57.17 μmol, 1.0 eq.) was added at 25° C. The resulting mixture was stirred at 25° C. for 0.5 hr. The reaction mixture was concentrated to yield a residue. The residue was purified by preparative HPLC (column: Boston Green ODS 150×30 mm×5 um; mobile phase: [water (FA)-ACN]; B %: 17%-47%, 14 min.), followed by lyophilization to yield I-281 (22.8 mg, 30.61 μmol, 53.54% yield.) was obtained as a white solid.

LC-MS (ESI+) m/z: 745.4 [M+H]⁺.

LCMS: calc. for C₄₆H₅₀F₂N₄O₃: 744.93, found: [M+H]⁺ 745.4.

HPLC: 100% purity at 220 nm ¹H NMR (400 MHZ, DMSO-d₆) δ ppm 10.74 (s, 1H), 9.63-9.86 (m, 1H), 8.16 (s, 1H), 7.17-7.23 (m, 3H), 7.07 (s, 1H), 6.97 (d, J=8.58 Hz, 2H), 6.83-6.91 (m, 4H), 6.55 (d, J=8.70 Hz, 2H), 6.44 (d, J=8.70 Hz, 2H), 6.12 (d, J=8.58 Hz, 2H), 4.29-4.34 (m, 1H), 3.67 (br dd, J=10.43, 4.95 Hz, 2H), 3.51 (br d, J=11.68 Hz, 2H), 3.44-3.47 (m, 1H), 3.42 (br d, J=7.03 Hz, 2H), 3.09-3.24 (m, 4H), 2.57-2.67 (m, 3H), 2.38-2.45 (m, 2H), 2.26 (br d, J=6.91 Hz, 2H), 2.05-2.14 (m, 1H), 1.97-2.03 (m, 1H), 1.85-1.96 (m, 2H), 1.69-1.79 (m, 4H), 1.45-1.56 (m, 1H), 1.01-1.16 (m, 4H).

### Example 52. Preparation of (I-282) 3-(4-((R*)-7-((1-(4-((1R,2S)-4,4-difluoro-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)piperidine-2,6-dione

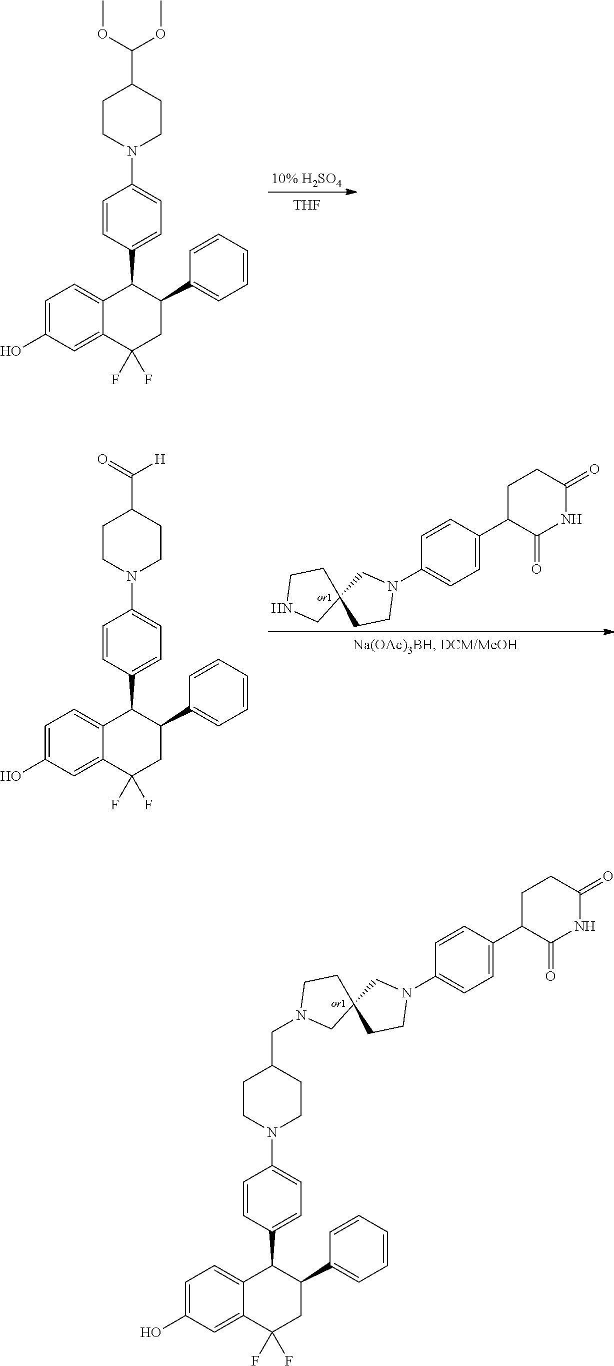

Step 1: To a solution of (5R,6S)-5-(4-(4-(dimethoxymethyl)piperidin-1-yl)phenyl)-8,8-difluoro-6-phenyl-5,6,7,8-tetrahydronaphthalen-2-ol (50 mg, 1 eq., 0.10 mmol) in THF (3 mL) was added H₂SO₄ (0.09 g, 0.05 mL, 9 eq., 0.9 mmol) and water (0.5 mL). The mixture was stirred at 40° C. for 0.5 h. The reaction mixture was quenched by addition of water (10 mL), adjust pH to 8 by NaHCO₃ saturated solution, extracted with EtOAc (20 mL×3). The combined organic layers were washed with brine (10 mL), dried over Na₂SO₄, filtered and concentrated under reduced pressure to give 1-(4-((1R,2S)-4,4-difluoro-6-hydroxy-2-phenyl-1,2,3,4-tetrahydronaphthalen-1-yl)phenyl)piperidine-4-carbaldehyde (40 mg, 89 μmol, 88% yield) as a yellow solid.

LC-MS (ESI⁺) m/z: 448.2 (M+H)⁺.

Step 2: To a solution of 1-(4-((1R,2S)-4,4-difluoro-6-hydroxy-2-phenyl-1,2,3,4-tetrahydronaphthalen-1-yl)phenyl)piperidine-4-carbaldehyde (40.0 mg, 89.4 μmol, 1.0 eq.) and 3-(4-((R)-2,7-diazaspiro[4.4]nonan-2-yl)phenyl)piperidine-2,6-dione (33.6 mg, 107 μmol, 1.2 eq.) in DCE (3 mL) and MeOH (3 mL) was added Sodium triacetoxyborohydride (75.8 mg, 358 μmol, 4.0 eq.). The mixture was stirred at 20° C. for 2 hour. LCMS showed the reaction was completed. The crude was purified by prep-HPLC together (Phenomenex Gemini NX 150×30 mm, 5 μm, water (water(NH₄HCO₃)-ACN) as a mobile phase, from 60% to 90%, Gradient Time (min): 11, Flow Rate (ml/min): 25) to give 3-(4-((R)-7-((1-(4-((1R,2S)-4,4-difluoro-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)piperidine-2,6-dione (27.6 mg, 37.1 μmol, 41.5%) as a white solid. LC-MS (ESI⁺) m/z: 745.4 (M+H)⁺.

LCMS: calc. for C₄₆H₅₀F₂N₄O₃: 744.93, found: [M+H]⁺ 745.4.

HPLC: 100.00% purity at 220 nm.

¹H NMR (400 MHz, METHANOL-d₄) δ: ppm 7.11-7.19 (m, 4H), 7.03 (d, J=8.40 Hz, 2H), 6.80-6.89 (m, 4H), 6.63 (d, J=8.80 Hz, 2H), 6.52 (d, J=8.40 Hz, 2H), 6.24 (d, J=8.80 Hz, 2H), 4.29 (d, J=2.80 Hz, 1H), 3.64-3.75 (m, 2H), 3.48-3.56 (m, 2H), 3.34 (s, 2H), 3.27 (d, J=9.40 Hz, 1H), 3.15-3.21 (m, 1H), 2.68 (s, 1H), 2.61-2.67 (m, 3H), 2.49-2.60 (m, 4H), 2.34-2.44 (m, 2H), 2.23-2.32 (m, 1H), 2.13-2.20 (m, 2H), 1.95-2.06 (m, 2H), 1.80-1.91 (m, 4H), 1.54-1.64 (m, 1H), 1.25-1.33 (m, 2H).

### Example 53. Preparation of (I-277) (S)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione

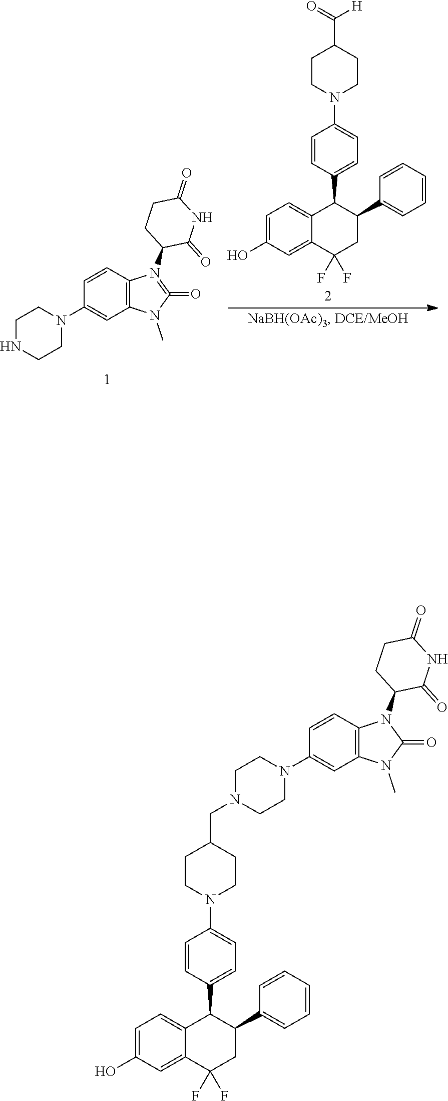

Step 1: To a solution of compound 1 (90.0 mg, 237 μmol, 1.0 eq.) in MeOH (2 mL) and DCE (3 mL) was added compound 2 (53.0 mg, 118 μmol, 0.5 eq.) at 25° C. After addition, the mixture was stirred at this temperature for 0.5 hr, and then NaBH₃(OAc)₃ (151.00 mg, 105 μL, 711 μmol, 3.0 eq.) was added at 25° C. The resulting mixture was stirred at 25° C. for 1 hr. The reaction mixture was concentrated to yield a residue. The residue was purified by preparative HPLC (column: Boston Green ODS 150×30 mm×5 um; mobile phase: [water (FA)-ACN]; B %: 13%-43%, 11 min.), followed by lyophilization to yield I-277 (46.1 mg, 59.5 μmol, 25.1%) was obtained as a white solid. LC-MS (ESI+) m/z: 775.3 [M+H]⁺.

LCMS: calc. for C₄₅H₄₈F₂N₆O₄: 774.91, found: [M+H]⁺ 775.3.

HPLC: 100% purity at 220 nm.

¹H NMR (400 MHz, DMSO-d₆) δ ppm 11.05 (s, 1H), 9.64-9.80 (m, 1H), 8.15 (s, 1H), 7.18-7.24 (m, 3H), 7.08 (s, 1H), 6.83-6.94 (m, 6H), 6.55-6.63 (m, 3H), 6.13 (d, J=8.58 Hz, 2H), 5.28 (dd, J=12.64, 5.25 Hz, 1H), 4.31 (br d, J=3.58 Hz, 1H), 3.46-3.71 (m, 4H), 3.30 (br s, 5H), 3.08 (br s, 4H), 2.84-2.93 (m, 1H), 2.52-2.74 (m, 6H), 2.34 (br d, J=13.59 Hz, 1H), 2.19 (brd, J=7.15 Hz, 2H), 1.94-2.02 (m, 1H), 1.57-1.77 (m, 3H), 1.09-1.20 (m, 2H).

### Example 54. Preparation of (I-278) (R)-3-(5-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione

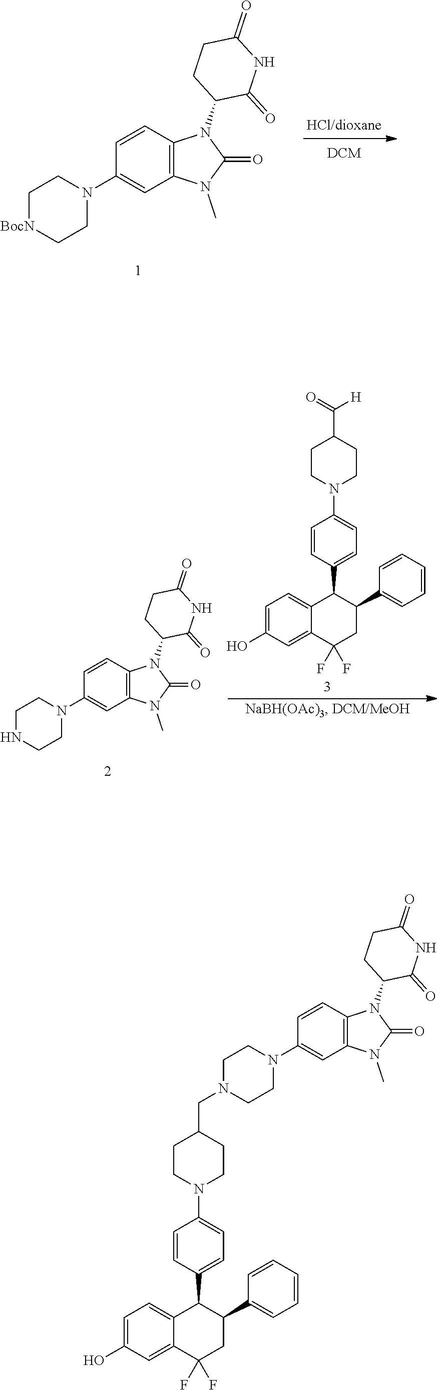

Step 1: To a mixture of compound 1 (80 mg, 0.18 mmol, 1 eq) in DCM (3 mL) was added HCl/dioxane (3 mL) at 25° C. The mixture was stirred at 25° C. for 1 h. LCMS (RLN-NB-2491-8-R1A1) showed the reaction was completed. The reaction mixture was concentrated to give compound 2 (60 mg, 80% purity, 77% yield) as a yellow solid, which was used to next step directly.

LC-MS (ESI⁺) m/z: 344.2 (M+H)⁺.

Step 2: To a mixture of compound 2 (50 mg, 0.15 mmol, 1 eq) in MeOH (3 mL) and DCE (3 mL) was added compound 3 (46 mg, 0.10 mmol, 0.7 eq) and NaBH(OAc)₃ (62 mg, 0.29 mmol, 2 eq) at 25° C. The mixture was stirred at 25° C. for 1 h. LCMS (RLN-NB-2491-10-R1A) showed the reaction was completed. The reaction mixture was concentrated to give a crude, which was purified by prep-HPLC (Column: Boston Green ODS 150×30 mm×5 um; Condition: water (FA)-ACN; Begin B: 13; End B: 43; Gradient Time (min): 12; 100% B Hold Time (min): 2; Flow Rate (mL/min): 25; injections: 8.) to give I-278 (41.0 mg, 36% yield) as a white solid. LC-MS (ESI⁺) m/z: 775.5 (M+H)⁺.

LCMS: calc. for C₄₅H₄₈F₂N₆O₄: 774.91, found: [M+H]⁺ 775.5.

HPLC: 100% purity at 220 nm.

¹H NMR: ¹H NMR (400 MHz, DMSO-d₆) δ=11.06 (s, 1H), 9.77-9.68 (m, 1H), 8.16 (s, 1H), 7.24-7.17 (m, 3H), 7.09 (s, 1H), 6.95-6.83 (m, 6H), 6.64-6.55 (m, 3H), 6.14 (d, J=8.7 Hz, 2H), 5.34-5.24 (m, 1H), 4.31 (br d, J=3.2 Hz, 1H), 3.70-3.63 (m, 1H), 3.57-3.50 (m, 2H), 3.30 (br s, 7H), 3.08 (br s, 4H), 2.95-2.85 (m, 1H), 2.75-2.56 (m, 4H), 2.47-2.26 (m, 2H), 2.20 (br d, J=7.2 Hz, 2H), 2.03-1.95 (m, 1H), 1.80-1.71 (m, 2H), 1.68-1.58 (m, 1H), 1.21-1.09 (m, 2H).

### Example 55. Preparation of (I-289) 1-(4-((R)-7-((1-(4-((1R,2S)-4,4-difluoro-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)dihydropyrimidine-2,4 (1H,3H)-dione

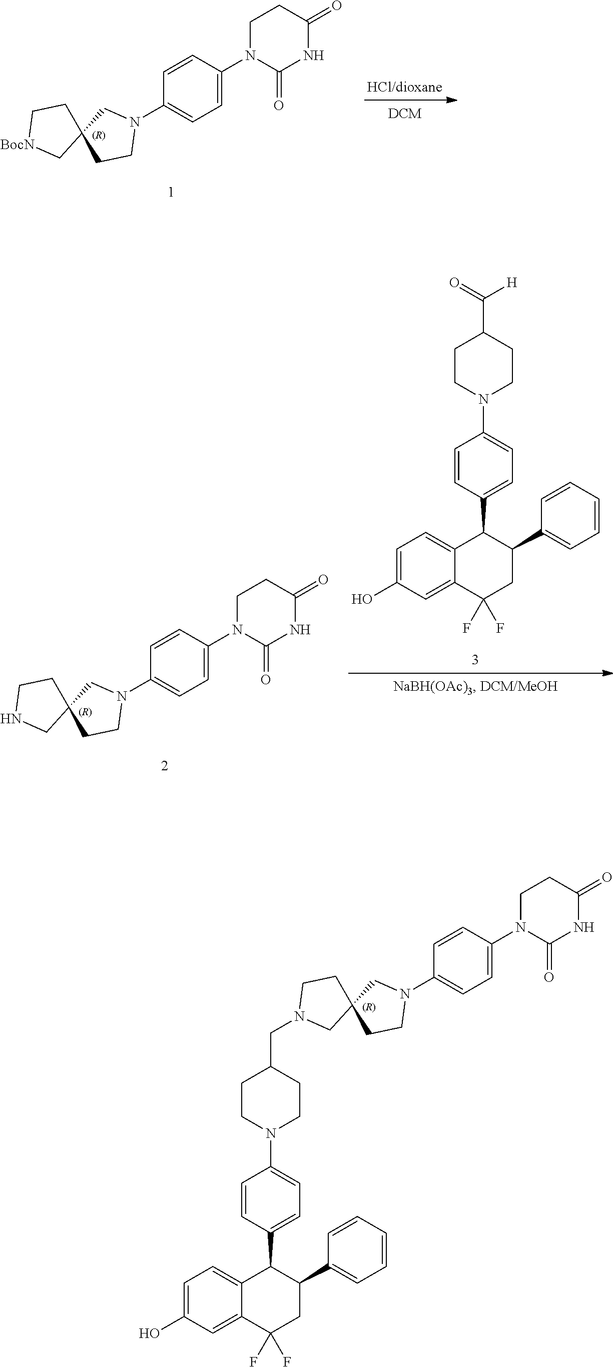

Step 1: To a mixture of compound 1 (100 mg, 241 μmol, 1 eq) in DCM (4 mL) was added HCl/dioxane (4 mL) at 25° C. The mixture was stirred at 25° C. for 1 h. LCMS (RLN-NB-2491-13-R1B) showed the reaction was completed. The reaction mixture was concentrated to give compound 2 (70 mg, 92% yield) as a yellow solid.

LC-MS (ESI⁺) m/z: 315.1 (M+H)⁺.

Step 2: To a mixture of compound 2 (70 mg, 0.22 mmol, 2 eq) in MeOH (3 mL) and DCE (3 mL) was added compound 3 (50 mg, 0.11 mmol, 1 eq) and NaBH(OAc)₃ (71 mg, 0.34 mmol, 3 eq) at 25° C. The mixture was stirred at 25° C. for 1 h. LCMS (RLN-NB-2491-14-R1A) showed the reaction was completed. The reaction mixture was concentrated to give a crude, which was purified by prep-HPLC (Column: Phenomenex Gemini NX 150×30 mm×5 um; Condition: water(NH₄HCO₃)-ACN; Begin B: 65; End B: 95; Gradient Time (min): 11; 100% B Hold Time (min): 2; Flow Rate (mL/min): 25; injections: 1.) to give 1-289 (17.9 mg, 21% yield) as a white solid. LC-MS (ESI⁺) m/z: 746.3 (M+H)⁺.

LCMS: calc. for C₄₅H₄₉F₂N₅O₃: 745.92, found: [M+H]⁺ 746.3.

HPLC: 99.33% purity at 220 nm.

¹H NMR: ¹H NMR (400 MHZ, METHANOL-d₄) δ=7.20-7.12 (m, 6H), 6.91-6.83 (m, 4H), 6.65 (d, J=8.7 Hz, 2H), 6.57 (d, J=8.9 Hz, 2H), 6.26 (d, J=8.7 Hz, 2H), 4.31 (br d, J=3.1 Hz, 1H), 3.79 (t, J=6.8 Hz, 2H), 3.74-3.67 (m, 1H), 3.58-3.52 (m, 2H), 3.38-3.35 (m, 2H), 3.25-3.21 (m, 1H), 2.80 (t, J=6.7 Hz, 3H), 2.75-2.63 (m, 3H), 2.61-2.53 (m, 3H), 2.46-2.27 (m, 3H), 2.09-2.00 (m, 2H), 1.92-1.84 (m, 4H), 1.67-1.58 (m, 1H), 1.41-1.24 (m, 3H).

### Example 56. Preparation of (I-227) (S)-3-(5-(4-((1-(4-((1R,2R)-2-((S)-1-cyclopropylethyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione and (I-228) (S)-3-(5-(4-((1-(4-((1R,2R)-2-((R)-1-cyclopropylethyl)-4,4-difluoro-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

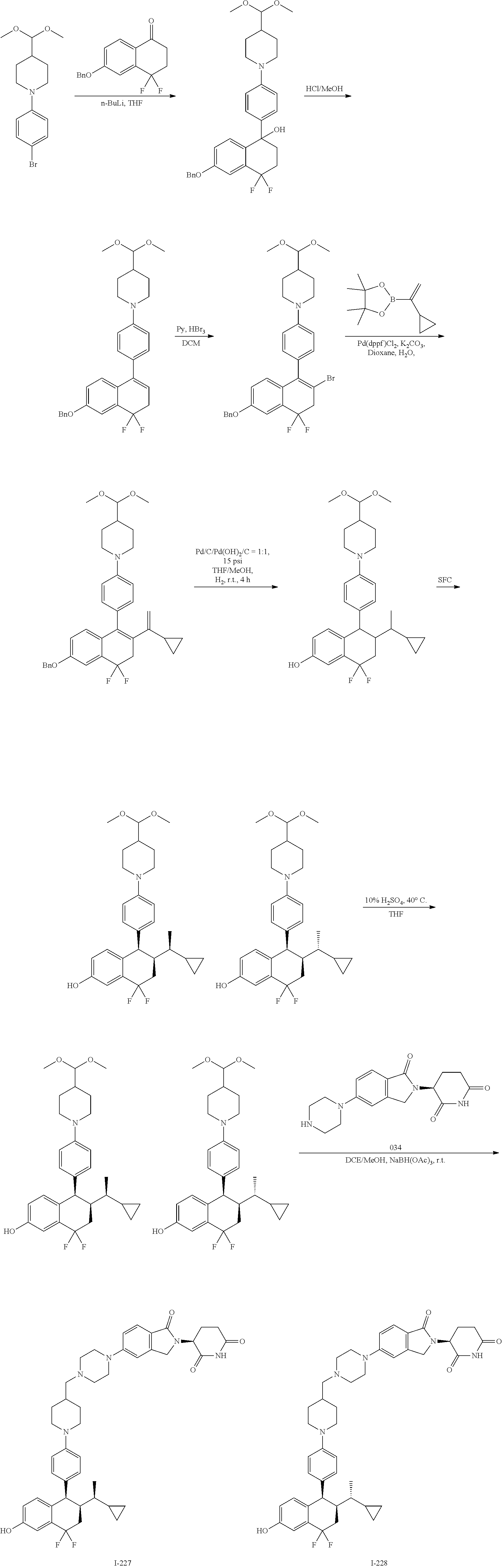

Step 1: To a solution of 1-(4-bromophenyl)-4-(dimethoxymethyl)piperidine (1.5 g, 4.774 mmol, 1.0 eq.) in THF (25 mL) was added dropwise n-butyllithium (2.3 mL, 5.728 mmol, 1.2 eq.) at −70° C. over 1 hour. After addition, the mixture was stirred at this temperature for 1 hour, and then 6-(benzyloxy)-4,4-difluoro-3,4-dihydronaphthalen-1 (2H)-one (1.651 g, 5.728 mmol, 1.2 eq.) in THE (25 mL) was added dropwise at 0° C. The resulting mixture was stirred at 0° C. for 12 hour. LCMS showed the reaction was completed. The reaction mixture was quenched by addition NH₄Cl at 20° C., and then diluted with water 30 mL and extracted with EA 90 mL (30 mL×3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=5/1) to give Compound 3 (1.5 g, 2.9 mmol, 60% yield) as a brown solid.

LC-MS (ESI⁺) m/z: 524.2 (M+H)⁺

Step 2: To a solution of 6-(benzyloxy)-1-(4-(4-(dimethoxymethyl)piperidin-1-yl)phenyl)-4,4-difluoro-1,2,3,4-tetrahydronaphthalen-1-ol (1.5 g, 1 eq., 2.9 mmol) in DCM (15 mL) was added into HCl/MeOH (15 mL, 4 M) at room temperature. The mixture was stirred at 20° C. for 1 hour. LCMS showed the reaction was completed. The reaction mixture was quenched by addition NaHCO₃ at 20° C., and then diluted with water 30 mL and extracted with EA 90 mL (30 mL×3). The combined organic layers were washed with EA 30 mL (10 mL×3), dried over Na₂SO₄, filtered and concentrated under reduced pressure to give 1-(4-(6-(benzyloxy)-4,4-difluoro-3,4-dihydronaphthalen-1-yl)phenyl)-4-(dimethoxymethyl)piperidine (1.2 g, 2.1 mmol, 75% yield, 90% Purity) as a brown solid.

LC-MS (ESI⁺) m/z: 506.2 (M+H)⁺

Step 3: To a solution of 1-(4-(6-(benzyloxy)-4,4-difluoro-3,4-dihydronaphthalen-1-yl)phenyl)-4-(dimethoxymethyl)piperidine (1.20 g, 2.37 mmol, 1.0 eq.) in DCM (20 mL) was added Pyridinium bromide perbromide (0.77 g, 2.37 mmol, 1.0 eq.). The mixture was stirred at 20° C. for 1 hour. LCMS showed the reaction was completed. The reaction mixture was quenched by addition water 30 mL at 20° C., and then diluted with water 30 mL and extracted with DCM 90 mL (30 mL×3). The combined organic layers were washed with DCM 30 mL (10 mL×3), dried over Na₂SO₄, filtered and concentrated under reduced pressure to give a residue. The crude material was purified on silica gel column chromatography (from pure PE to PE/EtOAc=5/1) to give Compound 5 (1.06 g, 1.86 mmol, 78% yield) as a yellow solid.

LC-MS (ESI⁺) m/z: 586.2 (M+H)⁺

Step 4: A mixture of 1-(4-(6-(benzyloxy)-2-bromo-4,4-difluoro-3,4-dihydronaphthalen-1-yl)phenyl)-4-(dimethoxymethyl)piperidine (1.30 g, 2.22 mmol, 1.0 eq.), 2-(1-cyclopropylvinyl)-4,4,5,5-tetramethyl-1,3,2-dioxaborolane (475 mg, 2.45 mmol, 1.1 eq.), PdCl₂(dppf) (163 mg, 222 μmol, 0.1 eq.) and K₂CO₃ (922 mg, 6.67 mmol, 3.0 eq.) in Dioxane (16 mL) and water (4 mL) was degassed and purged with N₂ for 3 times, and then the mixture was stirred at 90° C. for 2 hour under N₂ atmosphere. LC-MS showed the reaction was completed. The reaction mixture was quenched by addition EA 10 mL at 20° C., and then diluted with water 10 mL and extracted with EA 90 mL (30 mL×3). The combined organic layers were washed with EA 15 mL (5 mL×3), dried over Na₂SO₄, filtered and concentrated under reduced pressure to give a residue. The crude material was purified on silica gel column chromatography (from pure PE to PE/EtOAc=4/1) to give 1-(4-(6-(benzyloxy)-2-(1-cyclopropylvinyl)-4,4-difluoro-3,4-dihydronaphthalen-1-yl)phenyl)-4-(dimethoxymethyl)piperidine (300 mg, 0.47 mmol, 21% yield, 90% Purity) as a red solid.

LC-MS (ESI⁺) m/z: 572.4 (M+H)⁺

Step 5: To a solution of Compound 7 (0.300 g, 525 μmol, 1.0 eq.) in THF (3 mL) and MeOH (3 mL) was added Pd/C (27.9 mg, 262 μmol, 0.5 eq.) and Pd(OH) 2 (36.8 mg, 0.5 eq., 262 μmol) under N₂ atmosphere. The suspension was degassed and purged with H₂ for 3 times. The mixture was stirred under H₂ (15 Psi) at 40° C. for 5 hour. LCMS showed the reaction was completed. The crude was purified by prep. HPLC together (Boston Green ODS 150*30 mm*5 um, water (FA)-ACN as a mobile phase, from 75% to 100%, Gradient Time (min): 11, Flow Rate (ml/min): 25) to give 6-(1-cyclopropylethyl)-5-(4-(4-(dimethoxymethyl) piperidin-1-yl)phenyl)-8,8-difluoro-5,6,7,8-tetrahydronaphthalen-2-ol (60 mg, 0.12 mmol, 23% yield) as a white solid.

LC-MS (ESI⁺) m/z: 486.2 (M+H)⁺

Step 6: The compound 8 (90 mg, 0.19 mmol, 1.0 eq.) was purified by SFC (Column: DAICEL CHIRALCEL OJ (250 mm*30 mm, 10 um); Mobile phase: CO₂-EtOH (0.1% NH₃H₂O); from 20% to 20%; Flow rate: 70 mL/min) to give Compound 9 (45 mg, 93 μmol, 50% yield) and Compound 9A (45 mg, 93 μmol, 50% yield) both as white solid.

### Compound 9:

LC-MS (ESI+) m/z: 486.2 (M+H)+

### Compound 9A:

LC-MS (ESI+) m/z: 486.2 (M+H)+

Step 7: To a solution of Compound 9 (15 mg, 1 eq., 31 μmol) in THF (3 mL) was added H₂SO₄ (92 mg, 50 μL, 30 eq., 0.94 mmol) and water (0.5 mL). The mixture was stirred at 40° C. for 20 min. LCMS showed the reaction was completed. The mixture neutralized with 1N NaHCO₃ to pH=8. The mixture was extracted with EtOAc (15 mL×3). The combined organic layers were washed with brine (15 mL), dried over Na₂SO₄, filtered and concentrated to give Compound 10 (10 mg, 21 μmol, 67%) as a white solid.

LC-MS (ESI+) m/z: 440.3 (M+H)+

Step 8: To a solution of Compound 9A (15 mg, 1 eq., 31 μmol) in THF (3 mL) was added H₂SO₄ (92 mg, 50 μL, 30 eq., 0.94 mmol) and water (0.5 mL). The mixture was stirred at 40° C. for 20 min. LCMS showed the reaction was completed. The mixture neutralized with 1N NaHCO₃ to pH=8. The mixture was extracted with EtOAc (15 mL×3). The combined organic layers were washed with brine (15 mL), dried over Na₂SO₄, filtered and concentrated to give Compound 10A (10 mg, 21 μmol, 67%) as a white solid.

LC-MS (ESI+) m/z: 440.3 (M+H)+

Step 9: To a solution of Compound 10 (10.00 mg, 22.75 μmol, 1.0 eq.) and (S)-3-(1-oxo-5-(piperazin-1-yl)isoindolin-2-yl)piperidine-2,6-dione (7.471 mg, 22.75 μmol, 1.0 eq.) in DCE (3 mL) and MeOH (3 mL) was added Sodium triacetoxyborohydride (14.47 mg, 68.25 μmol, 3.0 eq.). The mixture was stirred at 20° C. for 2 hour. LCMS showed the reaction was completed. The crude was purified by prep-HPLC together (Phenomenex Gemini NX 150×30 mm, 5 um, water(NH₄HCO₃)-ACN) as a mobile phase, from 60% to 90%, Gradient Time (min): 11, Flow Rate (ml/min): 25) to give I-227 (6.00 mg, 7.98 μmol, 35.1%) as a white solid. LC-MS (ESI+) m/z: 752.4 (M+H)⁺

LCMS: calc. for C₄₄H₅₁F₂N₅O₄: 751.92, found: [M+H]⁺ 752.4.

HPLC: 97.69% purity at 220 nm.

¹H NMR (400 MHZ, METHANOL-d₄) δ: ppm 7.64 (d, J=8.80 Hz, 1H), 7.05-7.11 (m, 5H), 7.00 (d, J=9.60 Hz, 2H), 6.68 (m, 1H), 6.52 (d, J=8.40 Hz, 1H), 5.10 (m, 1H), 4.41 (d, J=6.40 Hz, 2H), 3.73-3.78 (m, 2H), 3.37-3.41 (m, 4H), 2.81-2.95 (m, 2H), 2.76-2.81 (m, 3H), 2.62-2.66 (m, 4H), 2.41-2.56 (m, 2H), 2.35 (d, J=6.80 Hz, 2H), 2.13-2.19 (m, 1H), 1.96 (d, J=12.80 Hz, 2H), 1.76-1.84 (m, 1H), 1.37-1.45 (m, 3H), 1.22-1.30 (m, 2H), 1.09-1.16 (m, 1H), 0.96-0.99 (m, 3H), 0.78 (t, J=7.20 Hz, 4H).

Step 10: To a solution of Compound 10A (10 mg, 23 μmol, 1.0 eq) and (S)-3-(1-oxo-5-(piperazin-1-yl)isoindolin-2-yl)piperidine-2,6-dione (8.2 mg, 25 μmol, 1.1 eq.) in DCE (3 mL) and MeOH (3 mL) was added Sodium triacetoxyborohydride (14 mg, 68 μmol, 3.0 eq.). The mixture was stirred at 20° C. for 2 hour. LCMS showed the reaction was completed. The crude was purified by prep-HPLC together (Phenomenex Gemini NX 150×30 mm, 5 um, water (water(NH₄HCO₃)-ACN) as a mobile phase, from 60% to 90%, Gradient Time (min): 11, Flow Rate (ml/min): 25) to give I-228 (8.2 mg, 11 μmol, 48%) as a white solid.

LC-MS (ESI+) m/z: 752.4 (M+H)⁺

LCMS: calc. for C₄₄H₅₁F₂N₅O₄: 751.92, found: [M+H]⁺ 752.4.

HPLC: 100.00% purity at 220 nm.

¹H NMR (400 MHz, METHANOL-d₄) δ: ppm 7.66 (d, J=8.40 Hz, 1H), 7.07-7.13 (m, 5H), 6.98-7.03 (m, 2H), 6.70 (m, 1H), 6.54 (d, J=8.40 Hz, 1H), 5.12 (m, 1H), 4.43 (d, J=6.00 Hz, 2H), 3.77 (d, J=10.40 Hz, 2H), 3.39-3.43 (m, 4H), 2.84-2.94 (m, 2H), 2.78-2.82 (m, 3H), 2.67 (s, 1H), 2.45-2.57 (m, 2H), 2.38 (d, J=6.80 Hz, 2H), 2.14-2.20 (m, 1H), 1.98 (d, J=12.40 Hz, 2H), 1.78-1.85 (m, 1H), 1.40-1.48 (m, 3H), 1.21-1.28 (m, 2H), 1.10-1.17 (m, 1H), 0.97-1.01 (m, 3H), 0.74-0.94 (m, 4H).

### Example 57. Preparation of (I-297) 3-(6-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione and (I-298) 3-(6-(4-((1-(4-((1R,2S)-4,4-difluoro-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione

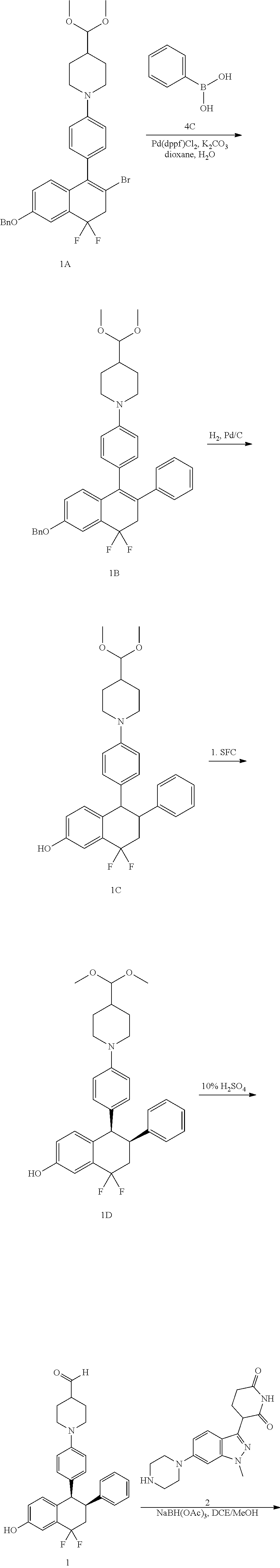

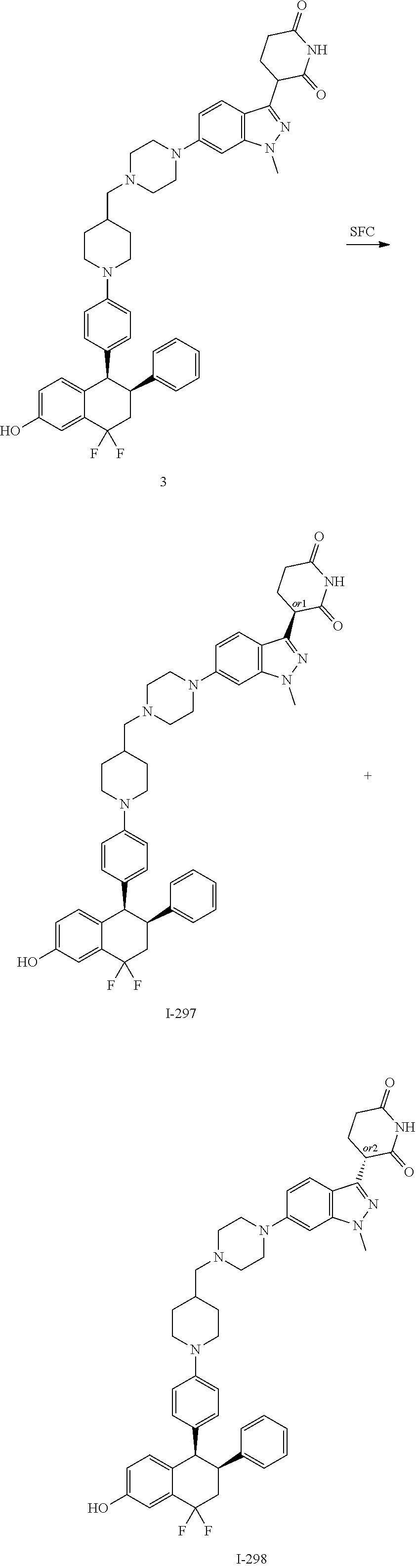 [SMILES: Cn1nc(C2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21 ; "SFC" ; Cn1nc([C@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21 ; Cn1nc([C@@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21]

Step 1: A mixture of compound 1A (873.00 mg, 1.77 mmol, 1.0 eq.), compound 4C (215.31 mg, 1.77 mmol, 1.0 eq.), K₂CO₃ (488.11 mg, 3.53 mmol, 2.0 eq.) and Pd(dppf)Cl₃ (115.09 mg, 176.59 μmol, 0.1 eq.) in dioxane (8 mL) and H₂O (2 mL) was degassed and purged with N₂ for 3 times, and then the mixture was stirred at 80° C. for 0.5 hr under N₂ atmosphere. LCMS showed that the starting material was consumed and the desired product was detected. The residue was purified by prep-HPLC (column: Boston Prime C18 150×30 mm×5 μm; mobile phase: water (NH₄HCO₃)-ACN; B %: 85%-100%, 10 min) to afford compound 1B (600.00 mg, 1.22 mmol, 69.12% yield) as a white solid.

LC-MS (ESI+) m/z: 582.2 (M+H)⁺.

Step 2: To a solution of compound 1B (1.5 g, 2.58 mmol, 1.0 eq.) in MeOH (3 ml) and THF (3 ml) was added Pd/C (1.37 g, 1.29 mmol, 10% purity, 0.5 eq.). The mixture was stirred under H₂ at 25° C. and 15 Psi for 4 hr. LCMS showed that the starting material was consumed and the desired product was detected. The reaction mixture was filtered under reduced pressure to give residue. The residue was purified by prep-HPLC (column: Boston Green ODS 150×30 mm×5 um; mobile phase: [water (TFA)-ACN]; B %: 50%-80%, 12 min.) to give compound 1C (1.00 g, 2.03 mmol, 78.57% yield) was obtained as a white solid.

LC-MS (ESI+) m/z: 494.5 (M+H)⁺.

Step 3: Compound 1C (840.00 mg) was separated by chiral SFC (Column: DAICEL CHIRALPAK AD (250 mm×30 mm, 10 um), Condition: CO₂-EtOH (0.1% NH₃H₂O), Begin B 30%, End B 30%, Flow Rate (ml/min): 70.) to give compound 1D (401.00 mg, 547.02 μmol, 45.00% yield).

LC-MS (ESI+) m/z: 494.3 (M+H)⁺.

Step 4: To a solution of compound 1D (80.00 mg, 0.16 mmol, 1.0 eq.) in THF (2 mL) was added H₂SO₄ (0.37 g, 0.20 mL, 10% Wt, 0.37 mmol, 2.3 eq.). The mixture was stirred at 25° C. for 0.5 br. LCMS showed that the starting material was consumed and the desired product was detected. The reaction liquid is dried with N₂ at 0° C. to give compound 1(65.00 mg, 0.15 mmol.) was obtained as yellow oil which was used in the next step without further purification.

LC-MS (ESI+) m/z: 448.2 (M+H)⁺.

Step 5: To a solution of compound 1 (100.00 mg, 275 μmol, 1.0 eq.) in MeOH (3 mL) and DCE (3 ml) was added compound 2 (61.5 mg, 137 μmol, 0.5 eq.) at 25° C. After addition, the mixture was stirred at this temperature for 0.5 hr, and then NaBH₃(OAc)₃ (58.2 mg, 40.7 μL, 275 μmol, 1.0 eq.) was added at 25° C. The resulting mixture was stirred at 25° C. for 3 hr. The reaction mixture was concentrated to yield a residue. The residue was purified by preparative HPLC (column: Boston Green ODS 150×30 mm×5 um; mobile phase: [water (FA)-ACN]; B %: 15%-45%, 12 min.), followed by lyophilization to yield compound 3 (120.00 mg, 158 μmol, 57.5%) was obtained as a white solid.

LC-MS (ESI+) m/z: 759.3 [M+H]⁺.

Step 6: Compound 3 (120.0 mg, 158.0 μmol, 1.0 eq.) was separated by chiral SFC (Column: DAICEL CHIRALPAK ID (250 mm×30 mm, 10 μm), Condition: MeOH-ACN Begin B 50% End B 50%, Begin B 25%, End B 25%, Flow Rate (mL/min): δ 0) to give I-297 (25.1 mg, 33.1 μmol, 20.9%) as a white solid and I-298 (23.1 mg, 30.4 μmol, 19.3%) as a white sold.

I-297: LC-MS (ESI+) m/z: 759.3 (M+H)⁺.

LCMS: calc. for C₄₅H₄₈F₂N₆O₃: 758.91, found: [M+H]⁺ 759.3.

HPLC: 100% purity at 220 nm

¹H NMR (400 MHz, DMSO-d₆) δ ppm 10.86 (s, 1H), 9.73 (s, 1H), 7.50 (d, J=8.94 Hz, 1H), 7.18-7.24 (m, 3H), 7.09 (s, 1H), 6.83-6.93 (m, 6H), 6.57 (d, J=8.70 Hz, 2H), 6.14 (d, J=8.70 Hz, 2H), 4.31 (br d, J=2.98 Hz, 1H), 4.26 (dd, J=9.18, 5.13 Hz, 1H), 3.89 (s, 3H), 3.67 (br d, J=13.83 Hz, 1H), 3.50-3.58 (m, 2H), 3.18-3.32 (m, 5H), 2.52-2.70 (m, 6H), 2.10-2.37 (m, 6H), 1.76 (br d, J=12.52 Hz, 2H), 1.61-1.70 (m, 1H), 1.07-1.28 (m, 3H).

I-298: LC-MS (ESI+) m/z: 759.3 (M+H)⁺.

LCMS: calc. for C₄₅H₄₈F₂N₆O₃: 758.91, found: [M+H]⁺ 759.3.

HPLC: 100% purity at 220 nm

¹H NMR (400 MHZ, DMSO-d₆) δ ppm 10.85 (s, 1H), 9.73 (s, 1H), 7.49 (d, J=8.94 Hz, 1H), 7.17-7.22 (m, 3H), 7.08 (s, 1H), 6.83-6.92 (m, 6H), 6.57 (d, J=8.58 Hz, 2H), 6.13 (d, J=8.58 Hz, 2H), 4.30 (br d, J=3.93 Hz, 1H), 4.25 (dd, J=9.18, 5.13 Hz, 1H), 3.88 (s, 3H), 3.63-3.69 (m, 1H), 3.50-3.56 (m, 2H), 3.21 (br s, 5H), 2.53-2.69 (m, 6H), 2.10-2.37 (m, 6H), 1.75 (br d, J=12.52 Hz, 2H), 1.59-1.69 (m, 1H), 1.09-1.26 (m, 3H).

### Example 58. Preparation of (I-4) (S)-3-(5-(4-((1-(4-((3R,4S)-7-hydroxy-3-phenylisochroman-4-yl)phenyl)piperidin-4-yl)methyl)piperazin-1-yl)-1-oxoisoindolin-2-yl)piperidine-2,6-dione

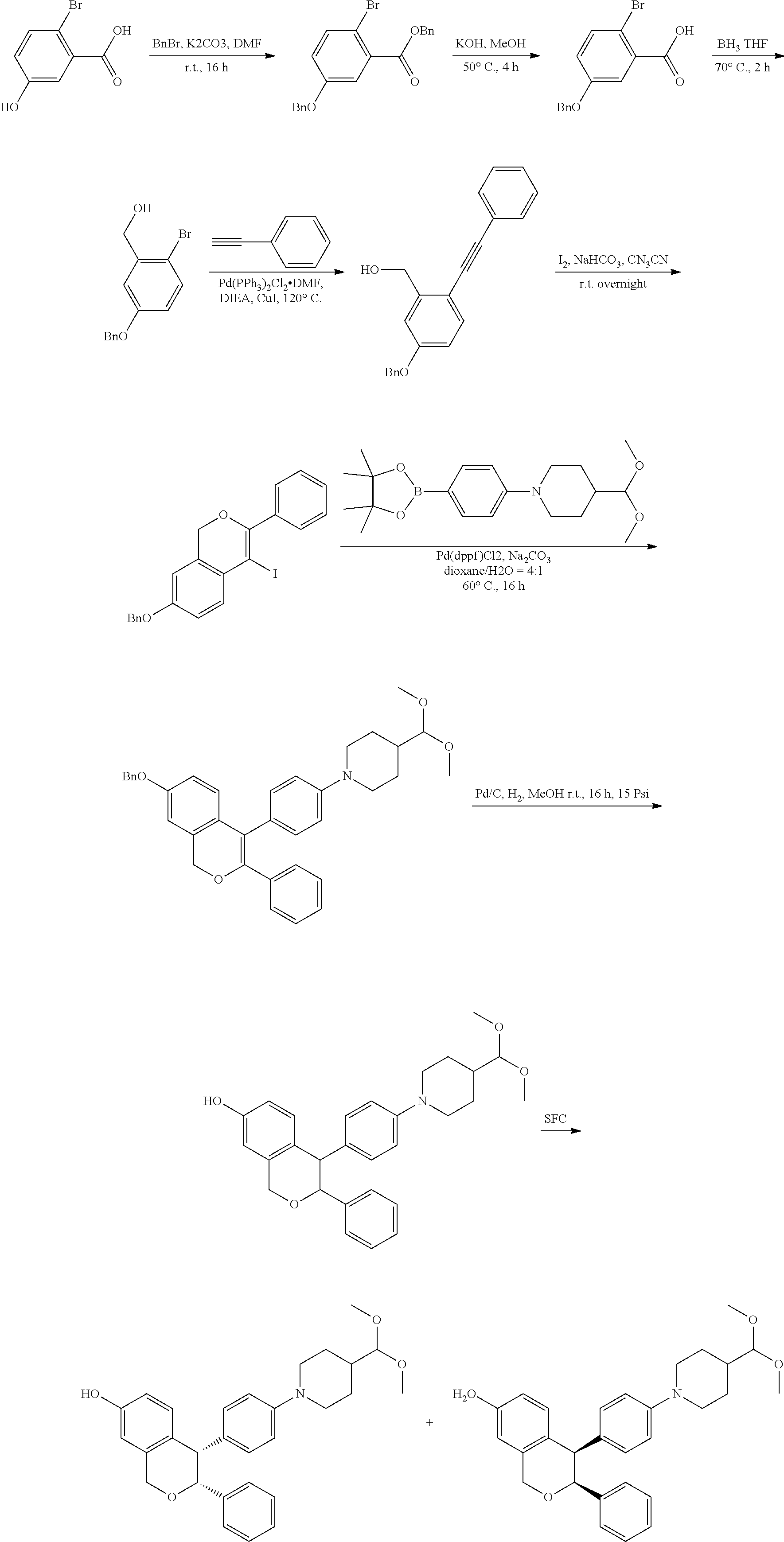

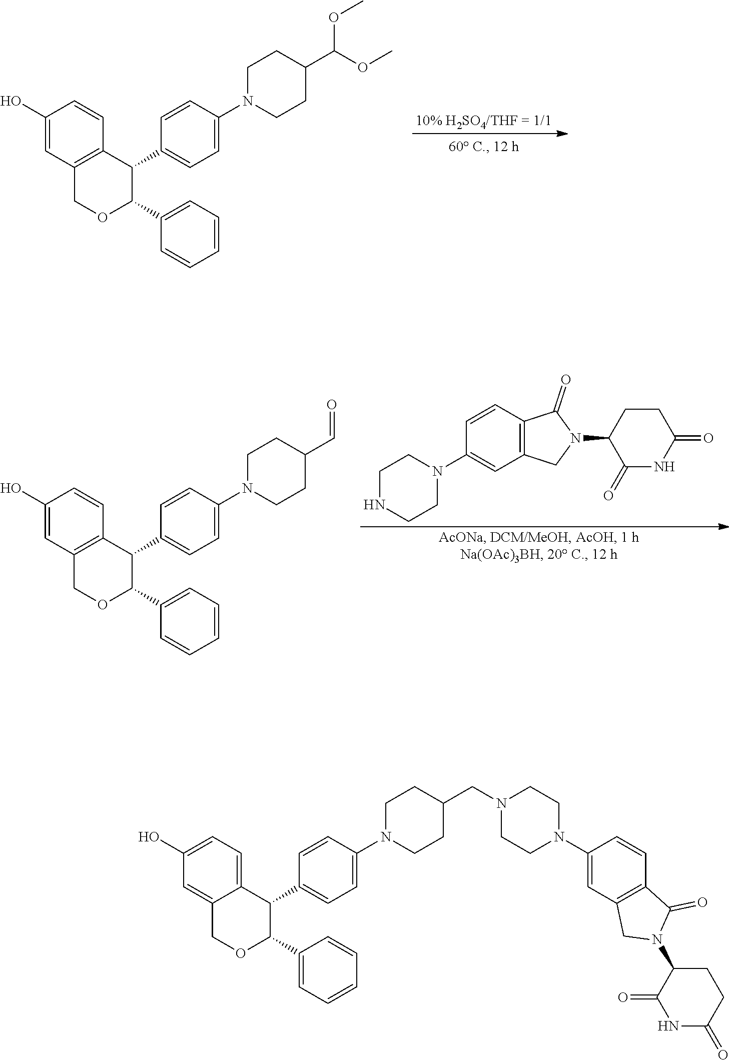

Step 1: To a solution of 2-bromo-5-hydroxybenzoic acid (1 g, 5 mmol,), K₂CO₃ (1 g, 10 mmol,) in DMF (15 mL) was added BnBr (2 g, 10 mmol,). The mixture was stirred at 25° C. for 16 hr. TLC (petroleum ether:ethyl acetate=5:1, R_{f}=0.3) indicated a new spot formed. The solvent was removed under vacuum. The residue was dissolved in water (30 mL) and extracted with ethyl acetate (2*15 mL). The combined organic layer was dried over Na₂SO₄, filtered and concentrated under vacuum to give benzyl 5-(benzyloxy)-2-bromobenzoate (1.7 g, 90% yield) as a yellow oil.

TLC: petroleum ether:ethyl acetate=5:1, R_{f}=0.3

Step 2: To a solution of benzyl 5-(benzyloxy)-2-bromobenzoate (1.7 g, 4.3 mmol) in MeOH (15 mL) was added KOH (1.3 mL, 13 mmol). The mixture was stirred at 50° C. for 4 hr. TLC (petroleum ether:ethyl acetate=3:1, R_{f}=0.3) indicated a new spot formed. The reaction was clean according to TLC. The mixture was dissolved in water (30 mL) and extracted with ethyl acetate (2*30 mL). The combined organic layer was dried over Na₂SO₄, filtered and concentrated under vacuum to give the compound as a yellow oil. The reaction was purified by flash column (petroleum ether/ethyl acetate from 100/0 to 60/40) and the organic layer was concentrated in vacuo to give 5-(benzyloxy)-2-bromobenzoic acid (1.2 g, 91% yield) as a yellow solid.

TLC: petroleum ether:ethyl acetate=3:1, R_{f}=0.3

Step 3: To a solution of 5-(benzyloxy)-2-bromobenzoic acid (1.2 g, 3.9 mmol) in THF (25 mL) was added dropwise BH₃ (1 M in THE, 9.8 mL, 9.8 mmol) at 0° C. over 30 min. After addition, the mixture was stirred at this temperature refluxed at 70° C. for 2 hr. TLC (petroleum ether:ethyl acetate=3:1, R_{f}=0.3) indicated a new spot formed. The reaction was clean according to TLC. The reaction mixture was quenched by addition MeOH at 0° C., and then extracted with EtoAc 30 mL (10 mL*3). The combined organic layers were dried over Na₂SO₄, filtered, and concentrated under reduced pressure to give a yellow solid. The reaction was purified by flash column (petroleum ether/ethyl acetate from 100/0 to 60/40) and the organic layer was concentrated in vacuo to give (5-(benzyloxy)-2-bromophenyl) methanol (900 mg, 75% yield) as a white solid.

TLC: petroleum ether:ethyl acetate=3:1, R_{f}=0.3

Step 4: To a mixture of (5-(benzyloxy)-2-bromophenyl) methanol (3 g, 1.0 eq., 0.01 mol), CuI (0.2 g, 0.03 mL, 0.1 eq., 1 mmol) in DMF (20 mL) was added bis-(triphenylphosphino)-palladous chloride (0.7 g, 0.1 eq., 1 mmol), DIEA (4 g, 5 mL, 3 eq., 0.03 mol) and ethynylbenzene (2 g, 2 mL, 1.5 eq., 0.02 mol). The mixture was stirred overnight at 120° C. for 12 hours under N₂. The mixture was added water (50 mL) and extracted with ethyl acetate (2*50 mL). The combined organic layer was dried over Na₂SO₄, filtered and concentrated under vacuum to give the compound as a yellow oil. The oil was purified by flash column (petroleum ether/ethyl acetate from 100/0 to 60/40) to give (5-(benzyloxy)-2-(phenylethynyl)phenyl) methanol (1.8 g, 4.5 mmol, 77.8% purity) as a yellow solid.

LC-MS (ESI⁺) m/z: 315.1 (M+H)⁺.

Step 5: To a solution of (5-(benzyloxy)-2-(phenylethynyl)phenyl) methanol (500 mg, 1 eq., 1.24 mmol) in MeCN (24 mL) was added sodium bicarbonate (312 mg, 3 Eq, 3.71 mmol) and iodine (942 mg, 3 eq., 3.71 mmol). The mixture was stirred at 25° C. for 12 hours under N₂. The mixture was added water (30 mL) and extracted ethyl acetate (2*30 mL). The combined organic layer was dried over Na₂SO₄, filtered and concentrated under vacuum to give the compound as a yellow oil. The oil was purified by flash column (petroleum ether/ethyl acetate from 100/0 to 90/10) and concentrated in vacuo to give 7-(benzyloxy)-4-iodo-3-phenyl-1H-isochromene (110 mg, 250 μmol, 20.2% yield) as a yellow solid.

¹H NMR (400 MHz, DMSO-d₆) δ=7.67-7.57 (m, 2H), 7.49-7.38 (m, 8H), 7.30-7.26 (m, 1H), 7.06-7.01 (m, 1H), 6.93-6.90 (m, 1H), 5.23-5.19 (m, 2H), 5.16-5.13 (m, 2H)

Step 6: A mixture of 1-(4-(6-(benzyloxy)-2-bromo-3,4-dihydronaphthalen-1-yl)phenyl)-4-(dimethoxymethyl)piperidine (600 mg, 1 eq., 1.09 mmol), (4-fluorophenyl)boronic acid (168 mg, 1.1 eq., 1.20 mmol), sodium carbonate (348 mg, 3 eq., 3.28 mmol), 1, l′-bis(di-t-butylphosphino) ferrocene palladium dichloride (71.3 mg, 0.1 eq., 109 μmol) in 1,4-dioxane (8 mL) and H₂O (2 mL) was degassed and purged with N₂ for 3 times, and then the mixture was stirred at 80° C. for 12 hour under N₂ atmosphere. The reaction mixture was quenched by addition 50 mL H₂O at 20° C., and extracted with ethyl acetate 50 mL (50 mL*2). The combined organic layers were washed with brine 50 mL, dried over Na₂SO₄, filtered and concentrated under reduced pressure to give a residue. The reaction was purified by flash column (petroleum ether/ethyl acetate from 100/0 to 60/40) and the organic layer was concentrated in vacuo to give 1-(4-(6-(benzyloxy)-2-(4-fluorophenyl)-3,4-dihydronaphthalen-1-yl)phenyl)-4-(dimethoxymethyl)piperidine (520 mg, 76.3% purity) as a yellow solid.

LC-MS (ESI⁺) m/z: 564.3 (M+H)⁺

TLC (petroleum ether/ethyl acetate=3/1, Rf=0.4) confirmed

Step 7: A mixture of 1-(4-(6-(benzyloxy)-2-(4-fluorophenyl)-3,4-dihydronaphthalen-1-yl)phenyl)-4-(dimethoxymethyl)piperidine (500 mg, 1 eq., 887 μmol), Pd/C (472 mg, 10% Wt) in MeOH (10 mL) was degassed and purged with N₂ for 3 times, and then the mixture was stirred at 25° C. for 12 hour under H₂ atmosphere (15 psi). The reaction mixture was filtered and concentrated to dryness in vacuo to give a white solid.

The white solid was subjected by SFC:
Column: DAICEL CHIRALCEL OD (250 mm*30 mm, 10 um)
Mobile phase: A: Supercritical CO₂, B: MEOH (0.1% NH₃H₂O), A:B=60:40 at 80 mL/min;
Column Temp: 38° C.;
Nozzle Pressure: 100 Bar;
Nozzle Temp: 60° C.;
Evaporator Temp: 20° C.;
Trimmer Temp: 25° C.;
Wavelength: 220 nm.

The aqueous phase was lyophilized to dryness to give (3R,4S)-4-(4-(4-(dimethoxymethyl)piperidin-1-yl)phenyl)-3-phenylisochroman-7-ol (164 mg, 345 μmol, 38.9%) and (3S,4R)-4-(4-(4-(dimethoxymethyl)piperidin-1-yl)phenyl)-3-phenylisochroman-7-ol (174 mg, 366 μmol, 41.2%) as a white solid.

LC-MS (ESI⁺) m/z: 476.2 (M+H)⁺

Step 8: A mixture of (3R,4S)-4-(4-(4-(dimethoxymethyl)piperidin-1-yl)phenyl)-3-phenylisochroman-7-ol (100 mg, 200.18 μmol, 92% purity, 1 eq) in 10% H₂SO₄ (1.5 mL) and THF (1.5 mL), then the mixture was stirred at 60° C. for 12 hr. The reaction mixture was diluted with H₂O (15 mL) and extracted with EtOAc (5 mL*3). The combined organic layers were washed with 15 mL brine, dried over Na₂SO₄, filtered and concentrated under reduced pressure to give 1-(4-((3R,4S)-7-hydroxy-3-phenylisochroman-4-yl)phenyl)piperidine-4-carbaldehyde (80 mg, 183.79 μmol, 91.81% yield, 95% purity) as a white solid.

LC-MS (ESI⁺) m/z: 414.2 (M+H)⁺

Step 9: A mixture of 1-(4-((3R,4S)-7-hydroxy-3-phenylisochroman-4-yl)phenyl)piperidine-4-carbaldehyde (50 mg, 114.87 μmol, 95% purity, 1 eq), HOAc (20.69 mg, 344.61 μmol, 19.71 uL, 3 eq), NaOAc (47.12 mg, 574.36 μmol, 5 eq) and (3S)-3-(1-oxo-5-piperazin-1-yl-isoindolin-2-yl)piperidine-2,6-dione (56.58 mg, 172.31 μmol, 1.5 eq, p-TSA salt) in DCM (1 mL) and MeOH (1 mL) was stirred at 20° C. for 1 h, NaCNBH₃ (48.69 mg, 229.74 μmol, 2 eq) was added to the reaction and then the mixture was stirred at 20° C. for 12 hr. The reaction mixture was diluted with 15 mL H₂O and extracted with EtOAc (5 mL*3). The combined organic layers were washed with 15 mL brine, dried over Na₂SO₄, filtered and concentrated under reduced pressure to give a yellow oil. The residue was purified by prep-HPLC (column: Welch Xtimate C18 150*30 mm*5 um; mobile phase: [water (FA)-ACN]; B %: 18%-48%, 7 min). The aqueous phase was lyophilized to dryness to give (S)-3-(5-(4-((1-(4-((3R,4S)-7-hydroxy-3-phenylisochroman-4-yl)phenyl)piperidin-4-yl)methyl)piperazin-1-yl)-1-oxoisoindolin-2-yl)piperidine-2,6-dione (32 mg, 44.08 μmol, 38.38% yield, 100% purity) as a white solid.

LC-MS (ESI+) m/z: 726.4 (M+H)⁺

HPLC: 100% purity at 220 nm

¹H NMR (400 MHZ, DMSO-d₆) δ=10.99-10.92 (m, 1H), 9.31-9.27 (m, 1H), 8.15-8.14 (m, 0.516H), 7.57-7.51 (m, 1H), 7.18-7.05 (m, 7H), 6.80-6.75 (m, 1H), 6.65-6.60 (m, 2H), 6.59-6.53 (m, 4H), 5.13-4.98 (m, 3H), 4.96-4.87 (m, 1H), 4.39-4.29 (m, 1H), 4.25-4.16 (m, 1H), 4.07-4.01 (m, 1H), 3.56-3.44 (m, 3H), 2.96-2.85 (m, 1H), 2.53 (br d, J=2.0 Hz, 9H), 2.44-2.22 (m, 4H), 2.01-1.91 (m, 1H), 1.81-1.61 (m, 3H), 1.25-1.08 (m, 2H).

### Example 59. Preparation of (I-3) (S)-3-(5-(4-((1-(4-((3S,4R)-7-hydroxy-3-phenylisochroman-4-yl)phenyl)piperidin-4-yl)methyl)piperazin-1-yl)-1-oxoisoindolin-2-yl)piperidine-2,6-dione

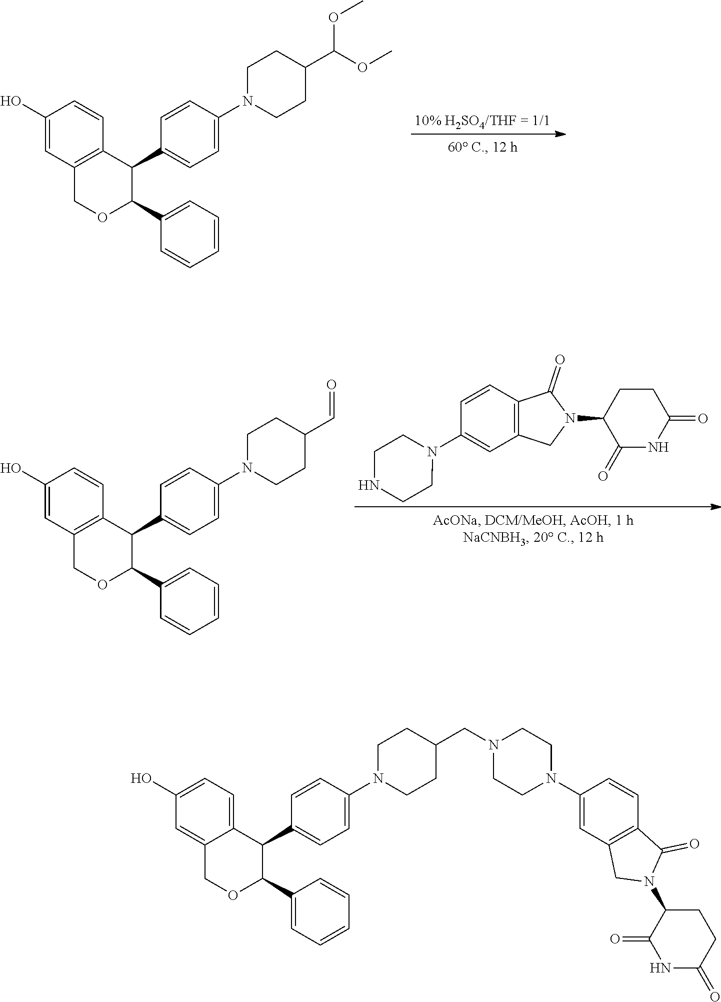 [SMILES: COC(OC)C1CCN(c2ccc([C@@H]3c4ccc(O)cc4CO[C@@H]3c3ccccc3)cc2)CC1 ; O=CC1CCN(c2ccc([C@@H]3c4ccc(O)cc4CO[C@@H]3c3ccccc3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccccc7)cc6)CC5)CC4)ccc3C2=O)C(=O)N1]

Step 1: A mixture of (3S,4R)-4-(4-(4-(dimethoxymethyl)piperidin-1-yl)phenyl)-3-phenylisochroman-7-ol (90 mg, 182.12 μmol, 93% purity, 1 eq) in 10% H₂SO₄ (1.5 mL) and THE (1.5 mL), then the mixture was stirred at 60° C. for 12 hr. The reaction mixture was diluted with 15 mL H₂O and extracted with EtOAc (5 mL*3). The combined organic layers were washed with brine 15 mL, dried over Na₂SO₄, filtered and concentrated under reduced pressure to give 1-(4-((3S,4R)-7-hydroxy-3-phenylisochroman-4-yl)phenyl)piperidine-4-carbaldehyde (80 mg, 181.86 μmol, 99.85% yield, 94% purity) as a white solid.

LC-MS (ESI+) m/z: 414.2 (M+H)⁺

Step 2: A mixture of 1-(4-((3S,4R)-7-hydroxy-3-phenylisochroman-4-yl)phenyl)piperidine-4-carbaldehyde (50 mg, 113.66 μmol, 94% purity, 1 eq), HOAc (20.48 mg, 340.99 μmol, 19.50 uL, 3 eq), NaOAc (46.62 mg, 568.31 μmol, 5 eq) and (3S)-3-(1-oxo-5-piperazin-1-yl-isoindolin-2-yl)piperidine-2,6-dione (55.98 mg, 170.49 μmol, 1.5 eq, p-TSA salt) in DCM (1 mL) and MeOH (1 mL) was stirred at 20° C. for 1 h, NaCNBH₃ (48.18 mg, 227.32 μmol, 2 eq) was added to the reaction and then the mixture was stirred at 20° C. for 12 hr. The reaction mixture was diluted with H₂O 15 mL and extracted with EtOAc (5 mL*3). The combined organic layers were washed with 15 mL brine, dried over Na₂SO₄, filtered and concentrated under reduced pressure to give a yellow oil. The residue was purified by prep-HPLC (column: Welch Xtimate C18 150*30 mm*5 um; mobile phase: [water (FA)-ACN]; B %: 16%-46%, 7 min). The aqueous phase was lyophilized to dryness to give (S)-3-(5-(4-((1-(4-((3S,4R)-7-hydroxy-3-phenylisochroman-4-yl)phenyl)piperidin-4-yl)methyl)piperazin-1-yl)-1-oxoisoindolin-2-yl)piperidine-2,6-dione (29.6 mg, 40.78 μmol, 35.88% yield, 100% purity) as a white solid.

LC-MS (ESI⁺) m/z: 726.4 (M+H)⁺

HPLC: 100% purity at 220 nm

¹H NMR (400 MHZ, DMSO-d₆) δ=10.98-10.93 (m, 1H), 9.34-9.27 (m, 1H), 8.18-8.12 (m, 0.331H), 7.56-7.50 (m, 1H), 7.19-7.03 (m, 7H), 6.83-6.75 (m, 1H), 6.66-6.59 (m, 2H), 6.59-6.51 (m, 4H), 5.12-4.98 (m, 3H), 4.95-4.87 (m, 1H), 4.39-4.28 (m, 1H), 4.24-4.17 (m, 1H), 4.09-3.99 (m, 1H), 3.63-3.42 (m, 3H), 2.97-2.83 (m, 1H), 2.53 (br d, J=2.0 Hz, 9H), 2.43-2.15 (m, 4H), 2.03-1.90 (m, 1H), 1.81-1.56 (m, 3H), 1.28-1.07 (m, 2H).

### Example 60. Preparation of (I-122) (S)-3-(5-(4-((1-(4-((S)-7′-hydroxyspiro[cyclohexane-1,3′-isochroman]-4′-yl)phenyl)piperidin-4-yl)methyl)piperazin-1-yl)-1-oxoisoindolin-2-yl)piperidine-2,6-dione

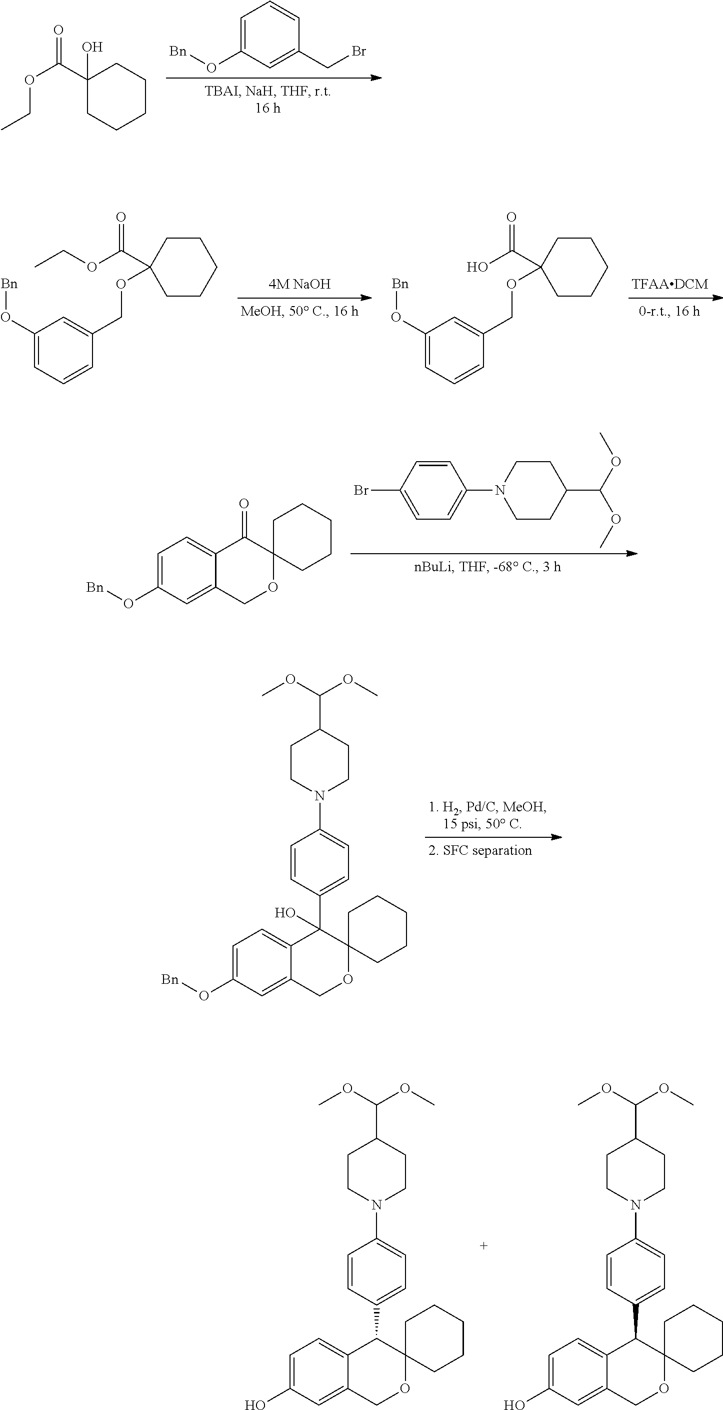

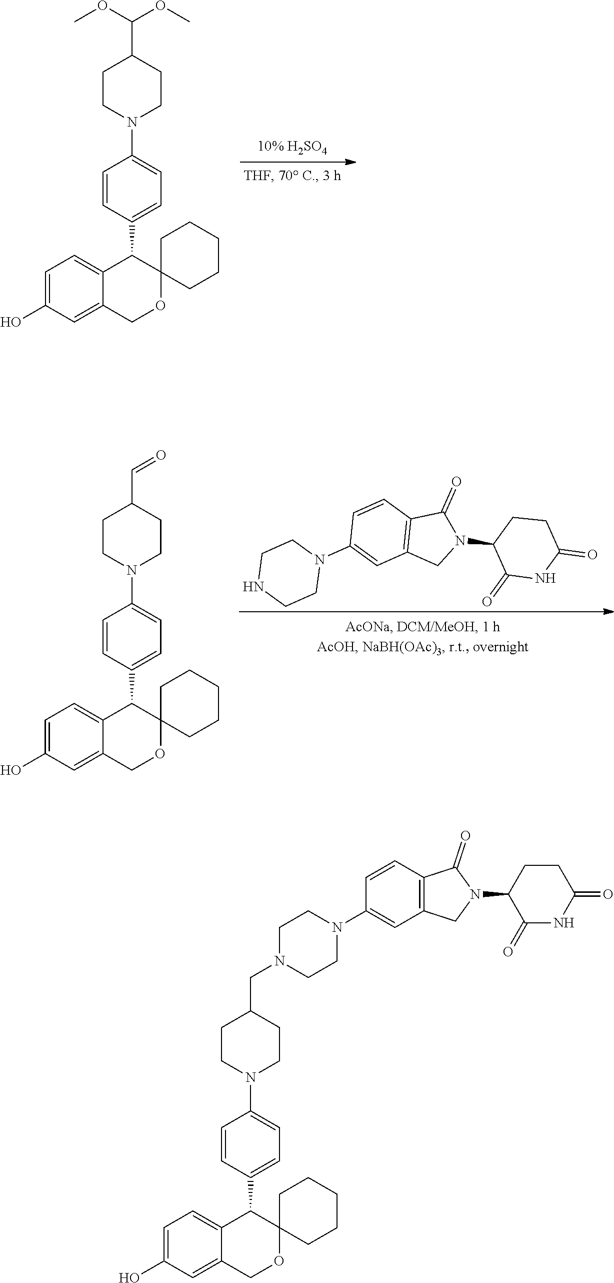 [SMILES: COC(OC)C1CCN(c2ccc([C@H]3c4ccc(O)cc4COC34CCCCC4)cc2)CC1 ; O=CC1CCN(c2ccc([C@H]3c4ccc(O)cc4COC34CCCCC4)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8COC78CCCCC8)cc6)CC5)CC4)ccc3C2=O)C(=O)N1]

Step 1: A mixture of ethyl 1-hydroxycyclohexane-1-carboxylate (8.00 g, 1 Eq, 46.5 mmol), 1-(benzyloxy)-3-(bromomethyl)benzene (19.3 g, 1.5 Eq, 69.7 mmol), Tetra-n-ButylammoniumIodide (1.72 g, 1.4 mL, 0.1 Eq, 4.65 mmol), sodium hydride (4.65 g, 3.9 mL, 60% Wt, 2.5 Eq, 116 mmol) in THF (100 mL) and DMF (10 mL) at 25° C. for 16 hour. TLC (petroleum ether:ethyl acetate=5:1, R_{f}=0.5 UV) showed one main new spot was observed. The reaction mixture was added 50 mL water at 0° C. and extracted with ethyl acetate (100 mL*3). The combined organic layers were washed with brine (100 mL*2), dried over anhydrous sodium sulfate, filtered and concentrated under reduced pressure. The residue was purified by flash silica gel chromatography (ISCO®; 20 g SepaFlash® Silica Flash Column, Eluent of 0˜15% Ethyl acetate/Petroleum ether gradient @ 45 mL/min) to give the product of ethyl 1-((3-(benzyloxy)benzyl)oxy)cyclohexane-1-carboxylate (13 g, 35 mmol, 76%).

Step 2: To a solution of ethyl 1-((3-(benzyloxy)benzyl)oxy)cyclohexane-1-carboxylate (12.00 g, 1 Eq, 32.57 mmol) in THF (50 mL) and MeOH (50 mL) was added NaOH (5.211 g, 32.57 mL, 4 M 4 Eq, 130.3 mmol). The mixture was stirred at 50° C. for 16 hour. TLC (petroleum ether/ethyl acetate=5:1, R_{f}=0.3) showed a new spot was formed. The mixture was adjusted to pH˜4 with 2N HCl. 100 mL H₂O was added, and the mixture was extracted with ethyl acetate (2×100 mL). The combined organic layers were washed with brine (100 mL), dried over anhydrous Na₂SO₄, filtered and concentrated to dryness in vacuo to give 1-((3-(benzyloxy)benzyl)oxy)cyclohexane-1-carboxylic acid (8 g, 0.02 mol, 70%) as yellow oil.

Step 3: 2,2,2-Trifluoroacetic anhydride (9.25 g, 6.12 mL, 5 Eq, 44.1 mmol) was added to 1-((3-(benzyloxy)benzyl)oxy)cyclohexane-1-carboxylic acid (3.00 g, 1 Eq, 8.81 mmol) in DCM (30 mL). The reaction was stirred at 0° C. for 30 min. The reaction was stirred at 25° C. for 16 hour. TLC (petroleum ether:ethyl acetate=10:1, UV) showed one main new spot was observed. 100 mL water was added, and the mixture was extracted with ethyl acetate (200 mL*2). The combined organic layers were washed with brine (100 mL), dried over anhydrous Na₂SO₄, filtered and concentrated to dryness in vacuo to give a white solid. The white solid was purified by flash column (petroleum ether/ethyl acetate from 100/0 to 0/100) and the organic layer was concentrated in vacuo to give 620 mg of 7′-(benzyloxy)spiro[cyclohexane-1,3′-isochroman]-4′-one as a yellow solid. 620 mg of 7′-(benzyloxy)spiro[cyclohexane-1,3′-isochroman]-4′-one was purified by preparative high-performance liquid chromatography. Condition: Column: O-Welch C18 150*30 mm*5 um A: water (FA) B: ACN; at the beginning: A (42%) and B (58%); at the end: A: (5%) and B (98%); Gradient Time (min) 10; 100% B hold Time (min) 2, Flow Rate (ml/min) 30. The pure fractions were collected, and the solvent was evaporated under vacuum. The aqueous layer was lyophilized to dryness to give 7′-(benzyloxy)spiro[cyclohexane-1,3′-isochroman]-4′-one (300 mg, 931 μmol, 10.6%) as a white solid.

Step 4:1-(4-bromophenyl)-4-(dimethoxymethyl)piperidine (584.78 mg, 2 Eq, 1.8610 mmol), a stir bar, and THF (10 mL) were added an oven-dried and nitrogen-purged 100 mL three round-bottomed flask, which was subsequently evacuated and refilled with argon (×3), before the reaction vessel was cooled to 25° C. (dry ice/EtOH). The resulting mixture treated with n-Butyllithium (89.41 mg, 558.31 μL, 2.5 M, 1.5 Eq, 1.3958 mmol), dropwise over 2 min at −68° C. (dry ice/EtOH), while stirring an additional 30 min at −68° C. (dry ice/EtOH). The resulting mixture treated with 7′-(benzyloxy)spiro[cyclohexane-1,3′-isochroman]-4′-one (300.000 mg, 1 Eq, 930.52 μmol) in THF (2 mL), dropwise over 5 min, and the reaction vessel removed to the r.t., while stirring an additional 2 h at −68° C. TLC (petroleum ether/ethyl acetate=5/1, R_{f}=0.4) The reaction mixture treated with sat. aq. NH₄Cl (20 mL), dropwise over 3 min, extracted with EtOAc (50 mL×3), and the combined extracts washed with brine (20 mL), dried over anhydrous Na₂SO₄, filtered and concentrated to dryness in vacuo to give a crude product, which was then subjected to silica gel chromatography (0-50% EtOAc/pet ether) to give 7′-(benzyloxy)-4′-(4-(4-(dimethoxymethyl)piperidin-1-yl)phenyl)spiro[cyclohexane-1,3′-isochroman]-4′-ol (300 mg, 538 μmol, 46.9%) as a yellow solid.

Step 5: A mixture of 7′-(benzyloxy)-4′-(4-(4-(dimethoxymethyl)piperidin-1-yl)phenyl)spiro[cyclohexane-1,3′-isochroman]-4′-ol (300 mg, 1 Eq, 538 μmol), Pd/C (286 mg, 10% Wt, 0.5 Eq, 269 μmol) in MeOH (10 mL) was degassed and purged with H₂ (15 psi) for 3 times, and then the mixture was stirred at 50° C. for 16 hour under H₂ atmosphere. LCMS showed desired MS was detected. The reaction mixture was filtered and concentrated under reduced pressure to give a residue. The residue was concentrated under reduced pressure to give desired compound (0.5 g, purity ˜90%) as a yellow oil, which was further separated by SFC (condition: column: DAICEL CHIRALCELOJ (250 mm*30 mm, 10 um); mobile phase: [0.1% NH₃—H₂O EtOH]; B %: 35%-35%, min) to give (S)-4′-(4-(4-(dimethoxymethyl)piperidin-1-yl)phenyl)spiro[cyclohexane-1,3′-isochroman]-7′-ol (110 mg, 244 μmol, 45.3%) was obtained as a yellow oil and (R)-4′-(4-(4-(dimethoxymethyl)piperidin-1-yl)phenyl)spiro[cyclohexane-1,3′-isochroman]-7′-ol (100 mg, 221 μmol, 43%).

Step 6: To a solution of (S)-4′-(4-(4-(dimethoxymethyl)piperidin-1-yl)phenyl)spiro[cyclohexane-1,3′-isochroman]-7′-ol (100 mg, 1 Eq, 221 μmol) in 10% H₂SO₄ (10 mL) and THF (10 mL). The mixture was stirred at 70° C. for 3 hour. TLC (petroleum ether:ethyl acetate=3:1, UV) showed one main new spot was observed. The reaction mixture was quenched by addition 50 mL H₂O at 25° C., and then diluted with 50 mL H₂O and extracted with Ethyl acetate 100 mL (50 mL*2). The combined organic layers were washed with 50 mL brine and dried over Na₂SO₄, filtered and concentrated under reduced pressure to give a residue. The reaction was no further purification for the next step.

Step 7: To a solution of (S)-1-(4-(7′-hydroxyspiro[cyclohexane-1,3′-isochroman]-4′-yl)phenyl)piperidine-4-carbaldehyde (70 mg, 1 Eq, 0.17 mmol), (S)-3-(1-oxo-5-(piperazin-1-yl)isoindolin-2-yl)piperidine-2,6-dione, TsOH salt (86 mg, 1 Eq, 0.17 mmol) in DCM (3 mL), MeOH (3 mL) was added Sodium acetate (42 mg, 3 Eq, 0.52 mmol) was stirred at 25° C. for 30 mins, Then Sodium triacetoxyborohydride (73 mg, 2 Eq, 0.35 mmol) and Acetic acid (31 mg, 30 μL, 3 Eq, 0.52 mmol) was added. The mixture was stirred at 25° C. for 16 hours. TLC (DCM/MeOH=10/1, R_{f}=0.4) showed new spot was formed. The reaction mixture was concentrated under reduced pressure to remove solvent. The residue was purified by prep-HPLC (column: Welch Xtimate C18 150*30 mm*5 um; mobile phase: [water (FA)-ACN]; B % 18%-48%, 7 min) to give (S)-3-(5-(4-((1-(4-((S)-7′-hydroxyspiro[cyclohexane-1,3′-isochroman]-4′-yl)phenyl)piperidin-4-yl)methyl)piperazin-1-yl)-1-oxoisoindolin-2-yl)piperidine-2,6-dione (25.1 mg, 35.0 μmol, 20%, 100% purity) was obtained as a white solid.

LCMS: calc. for C₄₃H₅₁N₅O₅: 717.39, found: [M+H]⁺ 718.2.

HPLC: 100.00% purity at 220 nm.

¹H NMR (400 MHZ, DMSO-d₆) δ=10.95 (s, 1H), 9.17 (br s, 1H), 8.16 (s, 0.29H), 7.52 (d, J=8.6 Hz, 1H), 7.11-7.02 (m, 2H), 6.92 (br s, 2H), 6.77 (d, J=8.9 Hz, 2H), 6.68 (d, J=8.3 Hz, 1H), 6.54-6.41 (m, 2H), 5.05 (dd, J=5.1, 13.3 Hz, 1H), 4.81-4.71 (m, 1H), 4.68-4.57 (m, 1H), 4.42-4.28 (m, 1H), 4.26-4.15 (m, 1H), 3.60 (br d, J=9.2 Hz, 2H), 3.51 (s, 1H), 3.29 (br s, 4H), 2.98-2.84 (m, 1H), 2.63-2.52 (m, 6H), 2.44-2.30 (m, 2H), 2.22 (br d, J=7.0 Hz, 2H), 2.03-1.91 (m, 1H), 1.80 (br d, J=11.7 Hz, 3H), 1.67 (br s, 1H), 1.56-1.33 (m, 5H), 1.31-1.14 (m, 4H), 1.09 (br s, 2H).

### Example 61. Preparation of (I-123) (S)-3-(5-(4-((1-(4-((R)-7′-hydroxyspiro[cyclohexane-1,3′-isochroman]-4′-yl)phenyl)piperidin-4-yl)methyl)piperazin-1-yl)-1-oxoisoindolin-2-yl)piperidine-2,6-dione

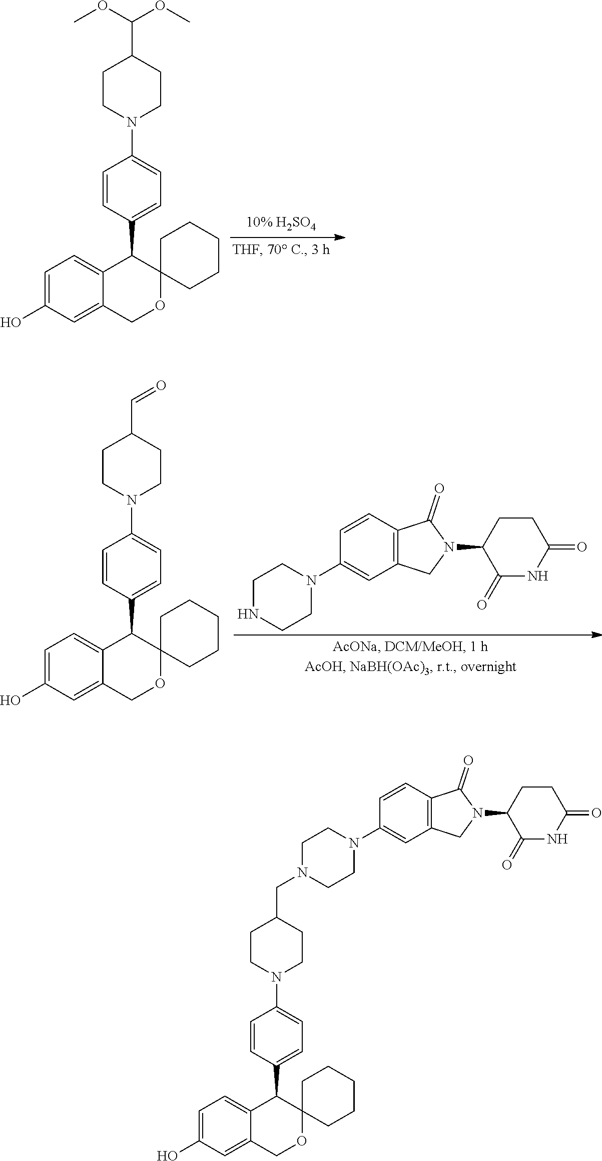

Step 1: To a solution of (R)-4′-(4-(4-(dimethoxymethyl)piperidin-1-yl)phenyl)spiro[cyclohexane-1,3′-isochroman]-7′-ol (100 mg, 1 Eq, 221 μmol) in 10% H₂SO₄ (10 mL) and THF (10 mL). The mixture was stirred at 70° C. for 3 hour. TLC (petroleum ether:ethyl acetate=3:1, UV) showed one main new spot was observed. The reaction mixture was quenched by addition 50 mL H₂O at 25° C., and then diluted with 50 mL H₂O and extracted with Ethyl acetate 100 mL (50 mL*2). The combined organic layers were washed with 50 mL brine and dried over Na₂SO₄, filtered and concentrated under reduced pressure to give a residue. The reaction was no further purification for next step.

Step 2: To a solution of (R)-1-(4-(7′-hydroxyspiro[cyclohexane-1,3′-isochroman]-4′-yl)phenyl)piperidine-4-carbaldehyde (60 mg, 1 Eq, 0.15 mmol), (S)-3-(1-oxo-5-(piperazin-1-yl)isoindolin-2-yl)piperidine-2,6-dione, TsOH salt (74 mg, 1 Eq, 0.15 mmol) in DCM (3 mL), MeOH (3 mL) was added Sodium acetate (36 mg, 3 Eq, 0.44 mmol) was stirred at 25° C. for 30 mins, Then Sodium triacetoxyborohydride (63 mg, 2 Eq, 0.30 mmol) and Acetic acid (27 mg, 26 μL, 3 Eq, 0.44 mmol) was added. The mixture was stirred at 25° C. for 16 hour. TLC (DCM/MeOH=10/1, R_{f}=0.4) showed new spot was formed. The reaction mixture was concentrated under reduced pressure to remove solvent. The residue was purified by prep-HPLC (column: Welch Xtimate C18 150*30 mm*5 um; mobile phase: [water (FA)-ACN]; B % 18%-48%, 7 min) to give (S)-3-(5-(4-((1-(4-((R)-7′-hydroxyspiro[cyclohexane-1,3′-isochroman]-4′-yl)phenyl)piperidin-4-yl)methyl)piperazin-1-yl)-1-oxoisoindolin-2-yl)piperidine-2,6-dione (29.8 mg, 41.5 μmol, 28%, 100% Purity) was obtained as a white solid.

LCMS: calc. for C₄₃H₅₁N₅O₅: 717.39, found: [M+H]⁺ 718.2.

HPLC: 100.00% purity at 220 nm.

¹H NMR (400 MHz, DMSO-d₆) δ=10.95 (s, 1H), 9.17 (s, 1H), 8.14 (s, 0.49H), 7.53 (d, J=8.7 Hz, 1H), 7.11-7.02 (m, 2H), 6.92 (br s, 2H), 6.77 (d, J=8.9 Hz, 2H), 6.68 (d, J=8.3 Hz, 1H), 6.53-6.42 (m, 2H), 5.05 (dd, J=5.1, 13.2 Hz, 1H), 4.80-4.71 (m, 1H), 4.69-4.59 (m, 1H), 4.39-4.29 (m, 1H), 4.26-4.14 (m, 1H), 3.60 (br d, J=9.7 Hz, 2H), 3.51 (s, 1H), 3.30-3.26 (m, 4H), 2.99-2.83 (m, 1H), 2.64-2.53 (m, 6H), 2.41-2.30 (m, 2H), 2.24 (br d, J=6.4 Hz, 2H), 2.02-1.91 (m, 1H), 1.80 (br d, J=12.2 Hz, 3H), 1.68 (br s, 1H), 1.56-1.35 (m, 5H), 1.31-1.14 (m, 4H), 1.09 (br s, 2H).

### Example 62. Preparation of (I-417) (S)-3-(5-(4-((1-(4-((3*R,4*R)-7-hydroxy-3-methyl-3-phenylisochroman-4-yl)phenyl)piperidin-4-yl)methyl)piperazin-1-yl)-1-oxoisoindolin-2-yl)piperidine-2,6-dione

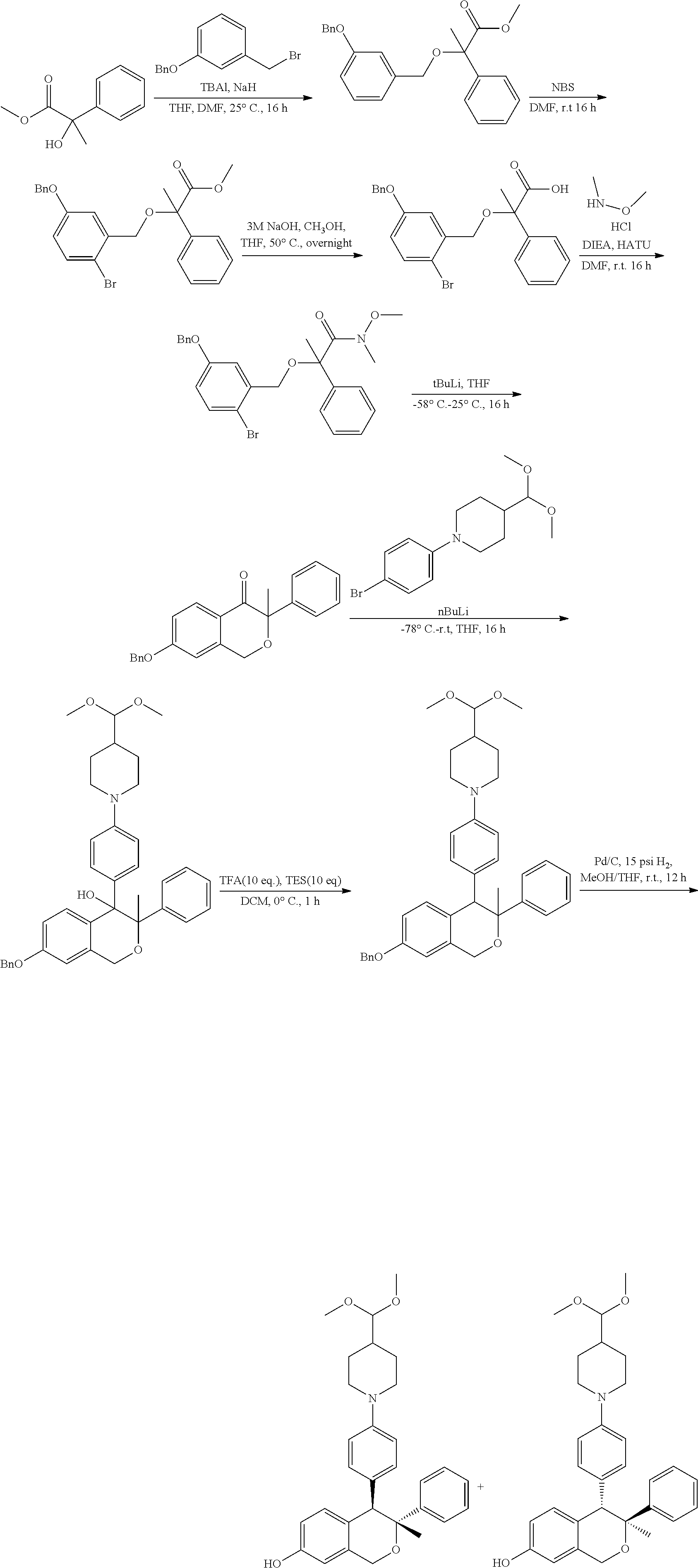

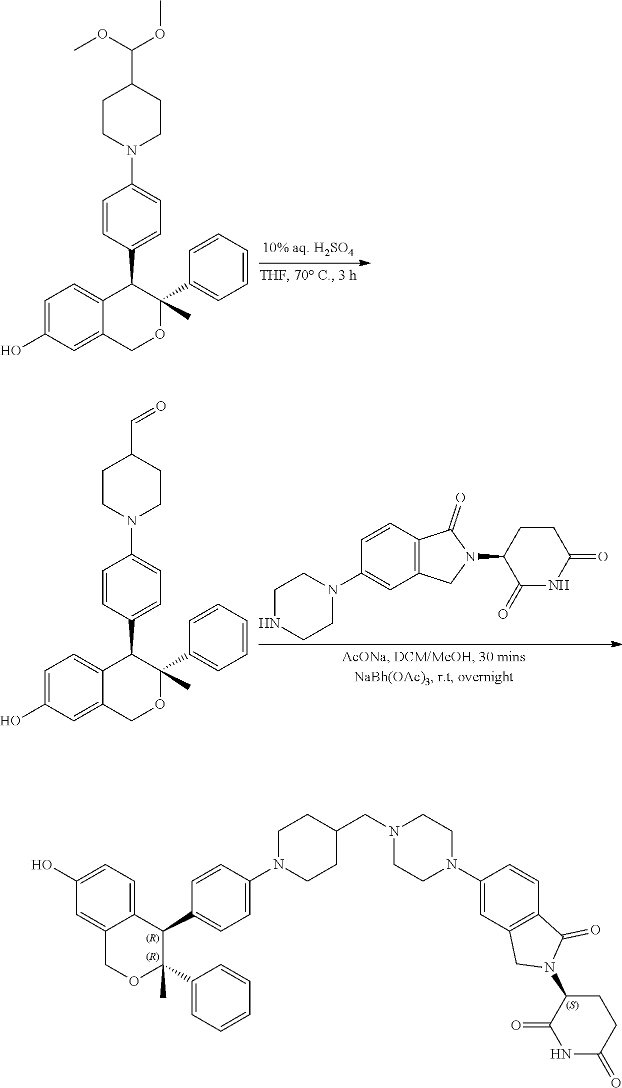

Step 1: A mixture of methyl 2-hydroxy-2-phenylpropanoate (4.9 g, 1 Eq, 27 mmol), 1-(benzyloxy)-3-(bromomethyl)benzene (11 g, 1.5 Eq, 41 mmol), TBAI (1.0 g, 0.1 Eq, 2.7 mmol) and sodium hydride (2.7 g, 60% Wt., 2.5 Eq, 68 mmol) in THF (50 mL) and DMF (20 mL) at 25° C. for 16 hour. LCMS showed 17.4% and 17.9% desired MS. The reaction mixture was added water 300 mL at 0° C. and extracted with ethyl acetate (500 mL*2). The combined organic layers were washed with brine, dried over anhydrous sodium sulfate, filtered and concentrated under reduced pressure. TLC (petroleum ether:ethyl acetate=1:1, UV) showed two main new spots was observed. The reaction was quenched with water (300 mL) and extracted with ethyl acetate (200 mL*3). The organic layer was washed with brine (50 mL) and dried over Na₂SO₄ and concentrated in vacuum to give a yellow oil. The yellow oil was subjected to column chromatography over silica gel (gradient elution: 0-100% EtOAc). LCMS showed 92.2% and 88.4% desired MS. The aqueous layer was lyophilized to dryness to give 2-((3-(benzyloxy)benzyl)oxy)-2-phenylpropanoic acid (1.9 g, 4.8 mmol, 18%, 92.199% purity) as a yellow oil. The aqueous layer was lyophilized to dryness to give methyl 2-((3-(benzyloxy)benzyl)oxy)-2-phenylpropanoate (2.65 g, 6.22 mmol, 23%, 88.4% purity) as a yellow oil. LC-MS (ESI⁺) m/z: 399.1 (M+H)⁺.

Step 2: A 40 mL thread vial was equipped with magnetic stirrer. A solution of methyl 2-((3-(benzyloxy)benzyl)oxy)-2-phenylpropanoate (2.65 g, 1 Eq, 7.04 mmol) in DMF (40 mL) and added N-Bromosuccinimide (1.50 g, 1.2 Eq, 8.45 mmol). The reaction was stirred at 25° C. for 16 hour. LCMS showed 33.2% desired MS. TLC (petroleum ether:ethyl acetate=10:1, UV) showed one main new spot was observed. The reaction was quenched with water (150 mL) and extracted with ethyl acetate (150 mL*3). The organic layer was washed with brine (50 mL) and dried over Na₂SO₄ and concentrated in vacuo to give methyl 2-((5-(benzyloxy)-2-bromobenzyl)oxy)-2-phenylpropanoate as a yellow oil. The yellow oil was subjected to column chromatography over silica gel (gradient elution: 0-10% EtOAc). LCMS showed 76% desired MS. The desired fractions were collected, and concentrated to dryness in vacuo to give methyl 2-((5-(benzyloxy)-2-bromobenzyl)oxy)-2-phenylpropanoate (3.2 g, 5.3 mmol, 76%, 76% purity) as a yellow oil. NMR confirmed. LC-MS (ESI⁺) m/z: 479.0 (M+H)⁺.

Step 3: A mixture of methyl 2-((5-(benzyloxy)-2-bromobenzyl)oxy)-2-phenylpropanoate (3.2 g, 76% Wt., 1 Eq, 5.3 mmol) and NaOH (1.1 g, 8.9 mL, 3 molar, 5 Eq, 27 mmol) in THF (10 mL) and MeOH (10 mL) at 50° C. for 16 hour. LCMS showed 71% desired MS. The reaction mixture was added 200 mL 1 N HCl at 0° C. and extracted with ethyl acetate (200 mL*3). The combined organic layers were washed with brine, dried over anhydrous sodium sulfate, filtered and concentrated under reduced pressure. TLC (petroleum ether:ethyl acetate=10:1, UV) showed two main new spots was observed. The reaction was quenched with water (300 mL) and extracted with ethyl acetate (200 mL*3). The organic layer was washed with brine (50 mL) and dried over Na₂SO₄ and concentrated in vacuo to give 2-((5-(benzyloxy)-2-bromobenzyl)oxy)-2-phenylpropanoic acid as a yellow oil. The yellow oil was subjected to column chromatography over silica gel (gradient elution: 0-15% EtOAc). LCMS showed 85.8% desired MS. The aqueous layer was lyophilized to dryness to give 2-((5-(benzyloxy)-2-bromobenzyl)oxy)-2-phenylpropanoic acid (1.65 g, 3.21 mmol, 60%, 85.8% purity) as a yellow oil. NMR confirmed. LC-MS (ESI+) m/z: 458.0 (M+H)⁺

Step 4: A 40 mL thread vial was equipped with magnetic stirrer. O,N-dimethyl-hydroxylamine HCl salt (375 mg, 1.2 Eq, 3.85 mmol) was added in to 2-((5-(benzyloxy)-2-bromobenzyl)oxy)-2-phenylpropanoic acid (1.65 g, 1 Eq, 3.21 mmol), DIEA (1.24 g, 1.66 mL, 3 Eq, 9.62 mmol) in DMF (15 mL) at r.t 30 min, and added into HATU (1.83 g, 1.5 Eq, 4.81 mmol). The vial was scaled with a rubber septum. The reaction was stirred at r.t. overnight. LCMS showed 20.08% desired MS. TLC (petroleum ether:ethyl acetate=5:1, UV) showed one main new spot was observed. The reaction was quenched with water (100 mL*2) and extracted with ethyl acetate (100 mL*3). The organic layer was washed with brine (50 mL) and dried over Na₂SO₄ and concentrated in vacuo to give 2-((5-(benzyloxy)-2-bromobenzyl)oxy)-N-methoxy-N-methyl-2-phenylpropanamide as a brown oil. The brown oil was subjected to column chromatography over silica gel (gradient elution: 0-20% EtOAc). The desired fractions were collected, and concentrated to dryness in vacuo to give 2-((5-(benzyloxy)-2-bromobenzyl)oxy)-N-methoxy-N-methyl-2-phenylpropanamide (1.9 g, 3.5 mmol, 89% purity) as a yellow oil. LC-MS (ESI+) m/z; 506.1 (M+H)⁺

Step 5: A three bottle equipped with magnetic stirrer. A solution of 2-((5-(benzyloxy)-2-bromobenzyl)oxy)-N-methoxy-N-methyl-2-phenylpropanamide (1.9 g, 1 Eq, 3.5 mmol) in THF (25 mL) was stirred at −78° C. for 30 min under N₂ atmosphere, then added tert-butyllithium (5.9 mL, 1.3 molar, 2.2 Eq, 7.7 mmol) at −78° C. The reaction was stirred at −78° C. to 25° C. for 16 hour under N₂ atmosphere. LCMS showed 65.9% desired MS. TLC (petroleum ether:ethyl acetate=5:1, UV) showed one main new spot was observed. The reaction was quenched with water (100 mL) and extracted with ethyl acetate (100 mL*3). The organic layer was washed with brine (50 mL) and dried over Na₂SO₄ and concentrated in vacuo to give 7-(benzyloxy)-3-methyl-3-phenylisochroman-4-one as a colorless oil. The black oil was subjected to column chromatography over silica gel (gradient elution: 0-20% EtOAc). The desired fractions were collected and concentrated to dryness in vacuo to give 7-(benzyloxy)-3-methyl-3-phenylisochroman-4-one (960 mg, 2.72 mmol, 78%, 97.47% purity) as a yellow solid. LC-MS (ESI+) m/z: 345.0 (M+H)⁺.

Step 6:1-(4-bromophenyl)-4-(dimethoxymethyl)piperidine (1.71 g, 2 Eq, 5.43 mmol), a stir bar, and THF (15 mL) were added an oven-dried and nitrogen-purged 100 mL three round-bottomed flask, which was subsequently evacuated and refilled with N₂ (×3), before the reaction vessel was cooled to −70° C. (dry ice/EtOH). The resulting mixture treated with n-Butyl lithium, 2.5 M in hexane (1.74 mL, 2.5 molar, 1.6 Eq. 4.35 mmol), dropwise over 2 min at −70° C. (dry ice/EtOH). While stirring an additional 1 hour at −70° C. (dry ice/EtOH). The resulting mixture treated with 7-(benzyloxy)-3-methyl-3-phenylisochroman-4-one (960 mg, 1 Eq, 2.72 mmol) in THF (5 mL), dropwise over 5 min, and the reaction vessel removed to the r.t., while stirring an additional 16 hour at 25° C. LCMS showed 68.565% desired MS. TLC (petroleum ether:ethyl acetate=5:1, UV) showed one main new spot was observed. The reaction was quenched with water (100 mL) and extracted with ethyl acetate (50 mL*3). The organic layer was washed with brine (50 mL) and dried over Na₂SO₄ and concentrated in vacuo to give 7-(benzyloxy)-4-(4-(4-(dimethoxymethyl)piperidin-1-yl)phenyl)-3-methyl-3-phenylisochroman-4-ol as a yellow oil. The yellow oil was subjected to column chromatography over silica gel (gradient elution: 0-20% EtOAc). The desired fractions were collected, and concentrated to dryness in vacuo to give 7-(benzyloxy)-4-(4-(4-(dimethoxymethyl)piperidin-1-yl)phenyl)-3-methyl-3-phenylisochroman-4-ol (1.5 g, 2.4 mmol, 87%, 91.73% purity) as a brown solid. LC-MS (ESI+) m/z: 580.3 (M+H)⁺

Step 7: An 8 mL thread vial was equipped with magnetic stirrer. 2,2,2-trifluoroacetic acid (531.02 mg, 345.9 μL, 9 Eq, 4.7 mmol) and triethylsilane (0.54 g, 0.85 mL, 10 Eq, 4.7 mmol) was added to a mixture suspension of 7-(benzyloxy)-4-(4-(4-(dimethoxymethyl)piperidin-1-yl)phenyl)-3-methyl-3-phenylisochroman-4-ol (300 mg, 1 Eq, 517.5 μmol) in DCM (5 mL) at 0° C. The mixture was stirred at 0° C. for 1 hour. LCMS showed 55.3% desired MS. TLC (petroleum ether:ethyl acetate=5:1, UV) showed one main new spot was observed. 50 mL water was added, and the mixture was extracted with dichloromethane (100 mL×2). The reaction was concentrated to dryness in vacuo to give a yellow oil. The yellow oil was purified by flash column (petroleum ether/ethyl acetate from 100/0 to 0/100) and the organic layer was concentrated in vacuo to give 1-(4-(7-(benzyloxy)-3-methyl-3-phenylisochroman-4-yl)phenyl)-4-(dimethoxymethyl) piper dine (100 mg, 155 μmol, 29.9%, 87.263% purity) as a yellow solid. LC-MS (ESI+) m/z: 564.3 (M+H)⁺.

Step 8: To a solution of 1-(4-(7-(benzyloxy)-3-methyl-3-phenylisochroman-4-yl)phenyl)-4-(dimethoxymethyl)piperidine (50 mg, 1 Eq, 77 μmol) in MeOH (1 mL) and THF (1 mL) was added Pd/C (82 mg, 10% Wt.) under N₂ atmosphere. The suspension was degassed and purged with H₂ for 3 mines. The mixture was stirred under 15 Psi H₂ (0.16 mg, 1 Eq, 77 μmol) at 50° C. for 16 hr. LCMS showed 58.629% desired MS was detected. The mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um)); mobile phase: [0.1% NH₃·H₂O EtOH]; B %: 55%-55%) to give (3*R,4*R)-4-(4-(4-(dimethoxymethyl)piperidin-1-yl)phenyl)-3-methyl-3-phenylisochroman-7-ol (10 mg, 21 μmol, 27%) and (3*S,4*S)-4-(4-(4-(dimethoxymethyl)piperidin-1-yl)phenyl)-3-methyl-3-phenylisochroman-7-ol (12 mg, 25 μmol, 33%) as a white solid LC-MS (ESI+) m/z: 474.3 (M+H)⁺.

Step 9:1-(4-((3*R,4*R)-7-hydroxy-3-methyl-3-phenylisochroman-4-yl)phenyl)piperidine-4-carbaldehyde (30 mg, 69 μmol) in sulfuric acid (3 mL, 10% Wt.,) and THF (3 mL) for stirred at 70° C. for 16 hour. LCMS showed 98.775% desired MS. The reaction was adjust to pH=˜7 with NaHCO₃, then 50 mL water was added, and the mixture was extracted with dichloromethane (10 mL×2). The combined organic layers were washed with brine (10 mL), dried over anhydrous Na₂SO₄, filtered and concentrated to dryness in vacuo to give a yellow oil. The yellow oil was concentrated in vacuo to give 1-(4-((3*R,4*R)-7-hydroxy-3-methyl-3-phenylisochroman-4-yl)phenyl)piperidine-4-carbaldehyde (30 mg, 69 μmol, 98.775% purity, crude) as a yellow solid. LC-MS (ESI+) m/z: 428.2 (M+H)⁺.

Step 10: To a solution of 1-(4-((3R,4R)-7-hydroxy-3-methyl-3-phenylisochroman-4-yl)phenyl)piperidine-4-carbaldehyde (30 mg, 98.775% Wt., 1 Eq, 69 μmol), (S)-3-(1-oxo-5-(piperazin-1-yl)isoindolin-2-yl)piperidine-2,6-dione, PhSO₃H salt (40 mg, 1.2 Eq, 83 μmol) in DCM (2 mL), MeOH (2 mL) was added sodium acetate (17 mg, 3 Eq, 0.21 mmol) was stirred at 25° C. for 30 mines, Then sodium triacetoxyborohydride (29 mg, 2 Eq, 0.14 mmol) and acetic acid (12 mg, 12 μL, 3 Eq, 0.21 mmol) was added. The mixture was stirred at 25° C. for 16 hour. LCMS showed 85.2% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 5%-45%, 8 min to give (S)-3-(5-(4-((1-(4-((3*R,4*R)-7-hydroxy-3-methyl-3-phenylisochroman-4-yl)phenyl)piperidin-4-yl)methyl)piperazin-1-yl)-1-oxoisoindolin-2-yl)piperidine-2,6-dione (13.6 mg, 18.4 μmol, 27%, 100% purity) was obtained as a white solid. LC-MS (ESI+) m/z: 740.2 (M+H)⁺.

LCMS: calc. for C₄₅H₄₉N₅₀₅, found: [M+H]⁺ 740.2.

HPLC: 100% purity at 220 nm.

¹H NMR 1H NMR (400 MHz, DMSO-d₆) δ=ppm 10.95 (s, 1H) 8.93-9.23 (m, 1H) 8.18 (s, 0.4H) 7.53 (d, J=8.70 Hz, 1H) 7.43 (d, J=7.51 Hz, 2H) 7.28 (t, J=7.63 Hz, 2H) 7.15-7.20 (m, 1H) 7.06 (br d, J=10.97 Hz, 4H) 6.83 (dd, J=10.43, 8.76 Hz, 3H) 6.46 (dd, J=8.46, 2.50 Hz, 1H) 6.26 (d, J=2.38 Hz, 1H) 5.05 (dd, J=13.29, 5.07 Hz, 1H) 4.82 (d, J=16.09 Hz, 1H) 4.50 (s, 1H) 4.30-4.45 (m, 2H) 4.18-4.25 (m, 1H) 3.63 (br d, J=11.68 Hz, 2H) 3.26-3.30 (m, 8H) 2.85-2.93 (m, 1H) 2.58-2.65 (m, 3H) 2.34-2.40 (m, 1H) 2.23 (br d, J=7.39 Hz, 2H) 1.95-2.01 (m, 1H) 1.81 (br d, J=11.92 Hz, 2H) 1.69 (br s, 1H) 1.22 (br d, J=12.99 Hz, 2H) 1.05 (s, 3H).

The absolute stereochemistry was arbitrarily assigned.

### Example 63. Preparation of (I-345) (S)-3-(5-(4-((1-(4-((3*S,4*S)-7-hydroxy-3-methyl-3-phenylisochroman-4-yl)phenyl)piperidin-4-yl)methyl)piperazin-1-yl)-1-oxoisoindolin-2-yl)piperidine-2,6-dione

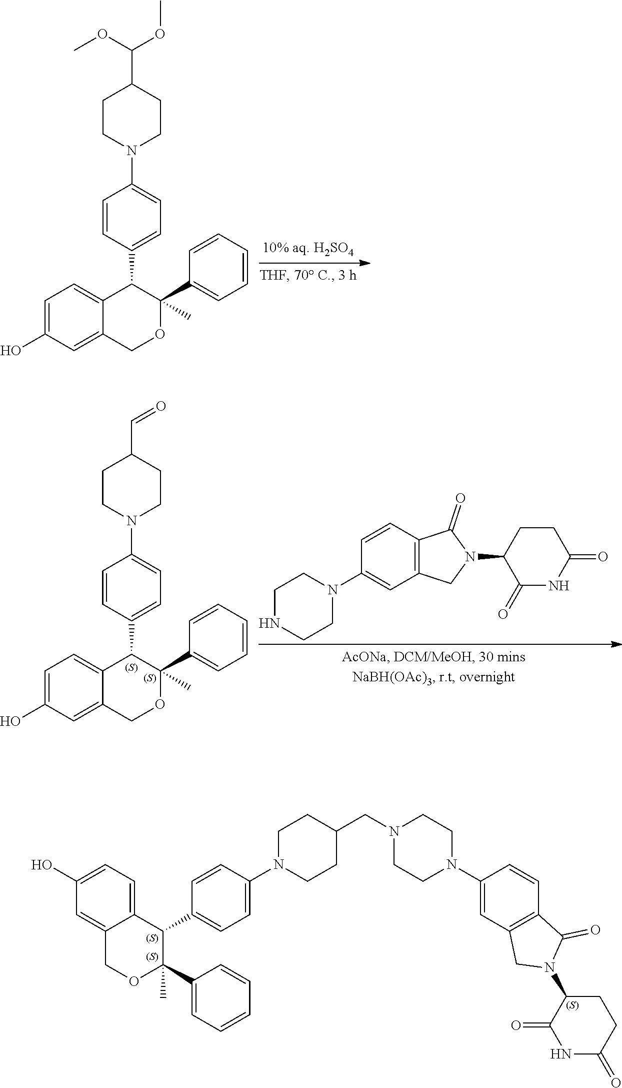

Step 1:1-(4-((3*S,4*S)-7-hydroxy-3-methyl-3-phenylisochroman-4-yl)phenyl)piperidine-4-carbaldehyde (32 mg, 74 μmol,) in sulfuric acid (58 mg, 1 mL, 10% Wt., 1 Eq, 59 μmol) and THF (3 mL) for stirred at 70° C. for 16 hour. LCMS showed 98.625% desired MS. The reaction was adjust to pH=˜7 with NaHCO₃, then 50 mL water was added, and the mixture was extracted with dichloromethane (100 mL×2). The combined organic layers were washed with brine (10 mL), dried over anhydrous Na₂SO₄, filtered and concentrated to dryness in vacuum to give a yellow oil. The yellow oil was concentrated in vacuum to give 1-(4-((3*S, 4*S)-7-hydroxy-3-methyl-3-phenylisochroman-4-yl)phenyl)piperidine-4-carbaldehyde (32 mg, 74 μmol, 98.625% purity) as a yellow solid. LC-MS (ESI+) m/z: 428.1 (M+H)⁺.

Step 2: To a solution of 1-(4-((3*S,4*S)-7-hydroxy-3-methyl-3-phenylisochroman-4-yl)phenyl)piperidine-4-carbaldehyde (30 mg, 1 Eq, 69 μmol), (S)-3-(1-oxo-5-(piperazin-1-yl)isoindolin-2-yl)piperidine-2,6-dione, PhSO₃H salt (40 mg, 1.2 Eq. 83 μmol) in DCM (2 mL), MeOH (2 mL) was added sodium acetate (17 mg, 3 Eq, 0.21 mmol) was stirred at 25° C. for 30 mins, Then sodium triacetoxyborohydride (29 mg, 2 Eq, 0.14 mmol) and acetic acid (12 mg, 12 μL, 3 Eq, 0.21 mmol) was added. The mixture was stirred at 25° C. for 16 hour. LCMS showed 83.584% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 5%-45%, 9 min to give (S)-3-(5-(4-((1-(4-((3*S,4*S)-7-hydroxy-3-methyl-3-phenylisochroman-4-yl)phenyl)piperidin-4-yl)methyl)piperazin-1-yl)-1-oxoisoindolin-2-yl)piperidine-2,6-dione (13.2 mg, 17.8 μmol, 26%, 100% purity) was obtained as a white solid. LC-MS (ESI+) m/z: 740.2 (M+H)⁺.

LCMS: calc. for C₄₅H₄₉N₅₀₅, found: [M+H]⁺ 740.2.

HPLC: 100% purity at 220 nm.

¹H NMR 1H NMR (400 MHZ, DMSO-d₆) δ=ppm 10.95 (s, 1H) 9.11 (br s, 1H) 8.18 (s, 0.648H) 7.53 (d, J=8.70 Hz, 1H) 7.43 (d, J=7.51 Hz, 2H) 7.28 (t, J=7.63 Hz, 2H) 7.14-7.22 (m, 1H) 7.06 (br d, J=10.85 Hz, 4H) 6.83 (dd, J=10.31, 8.76 Hz, 3H) 6.46 (dd, J=8.23, 2.50 Hz, 1H) 6.26 (d, J=2.27 Hz, 1H) 5.05 (dd, J=13.23, 5.13 Hz, 1H) 4.82 (d, J=16.09 Hz, 1H) 4.50 (s, 1H) 4.30-4.43 (m, 2H) 4.17-4.25 (m, 1H) 3.63 (br d, J=12.28 Hz, 2H) 3.27-3.30 (m, 8H) 2.88-2.96 (m, 1H) 2.59 (br d, J=13.35 Hz, 3H) 2.37 (br dd, J=13.11, 4.41 Hz, 1H) 2.23 (br d, J=7.27 Hz, 2H) 1.93-2.01 (m, 1H) 1.81 (br d, J=12.40 Hz, 2H) 1.69 (br s, 1H) 1.22 (br d, J=13.47 Hz, 2H) 1.05 (s, 3H).

The absolute stereochemistry was arbitrarily assigned.

### Example 64. Preparation of (I-34) (S)-3-(5-(4-((1-(4-((3R,4R)-3-cyclohexyl-7-hydroxyisochroman-4-yl)phenyl)piperidin-4-yl)methyl)piperazin-1-yl)-1-oxoisoindolin-2-yl)piperidine-2,6-dione

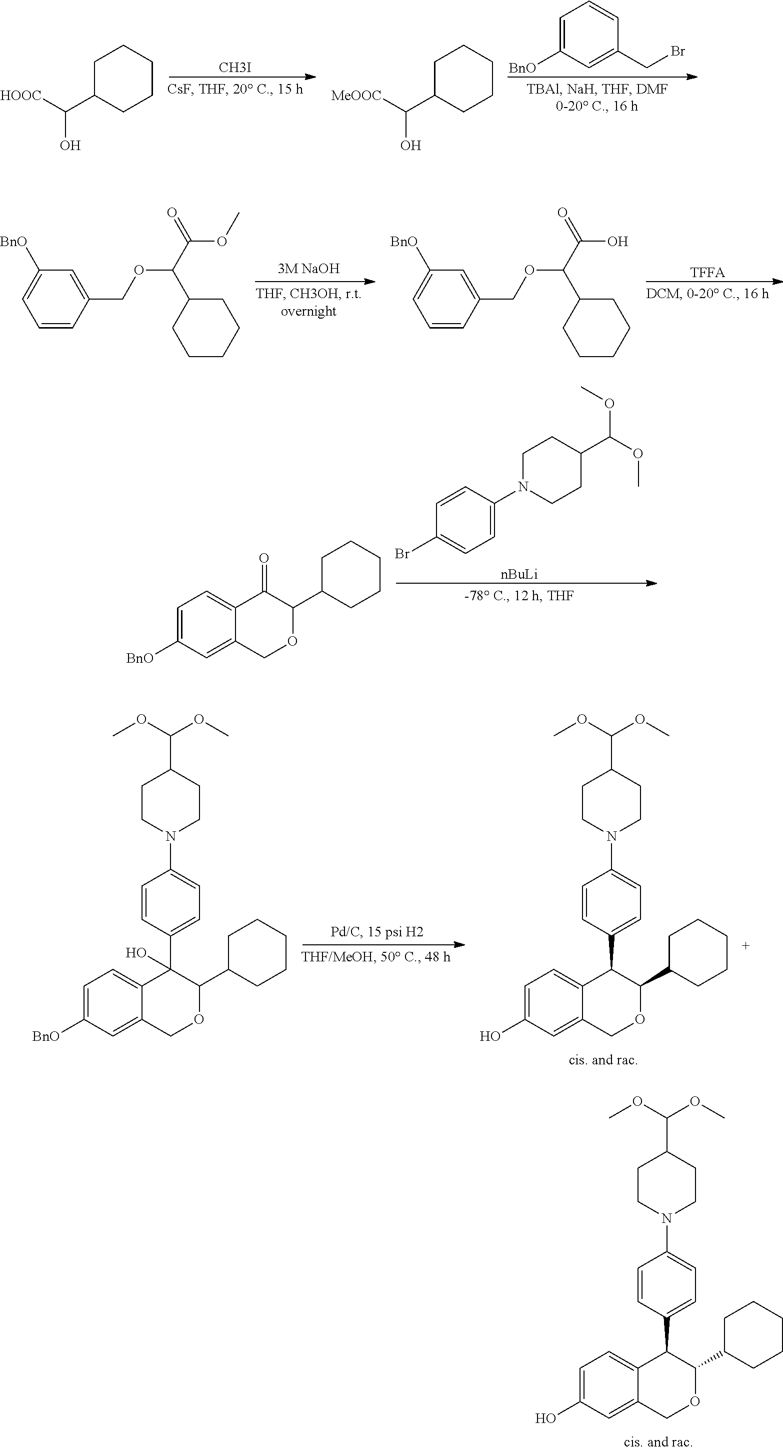

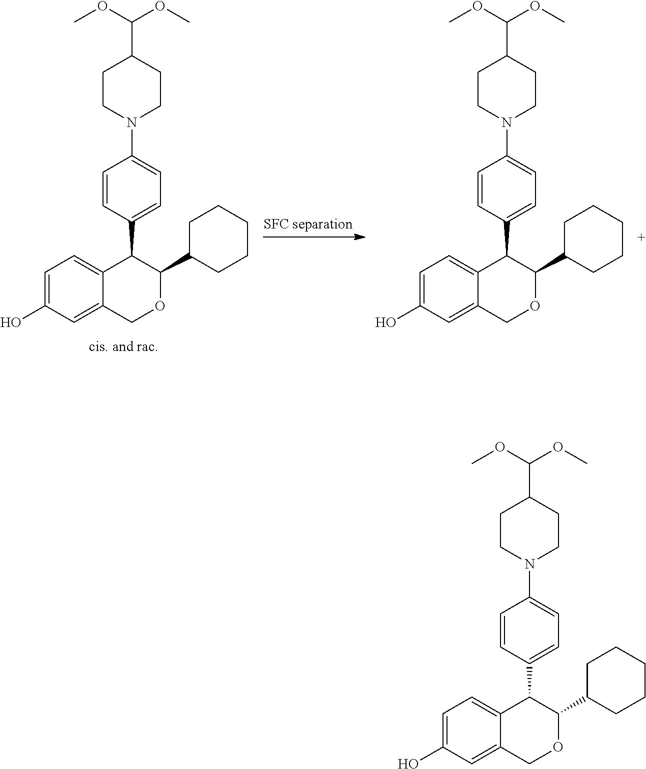 [SMILES: COC(OC)C1CCN(c2ccc([C@@H]3c4ccc(O)cc4CO[C@@H]3C3CCCCC3)cc2)CC1 ; COC(OC)C1CCN(c2ccc([C@@H]3c4ccc(O)cc4CO[C@@H]3C3CCCCC3)cc2)CC1 ; COC(OC)C1CCN(c2ccc([C@H]3c4ccc(O)cc4CO[C@H]3C3CCCCC3)cc2)CC1]

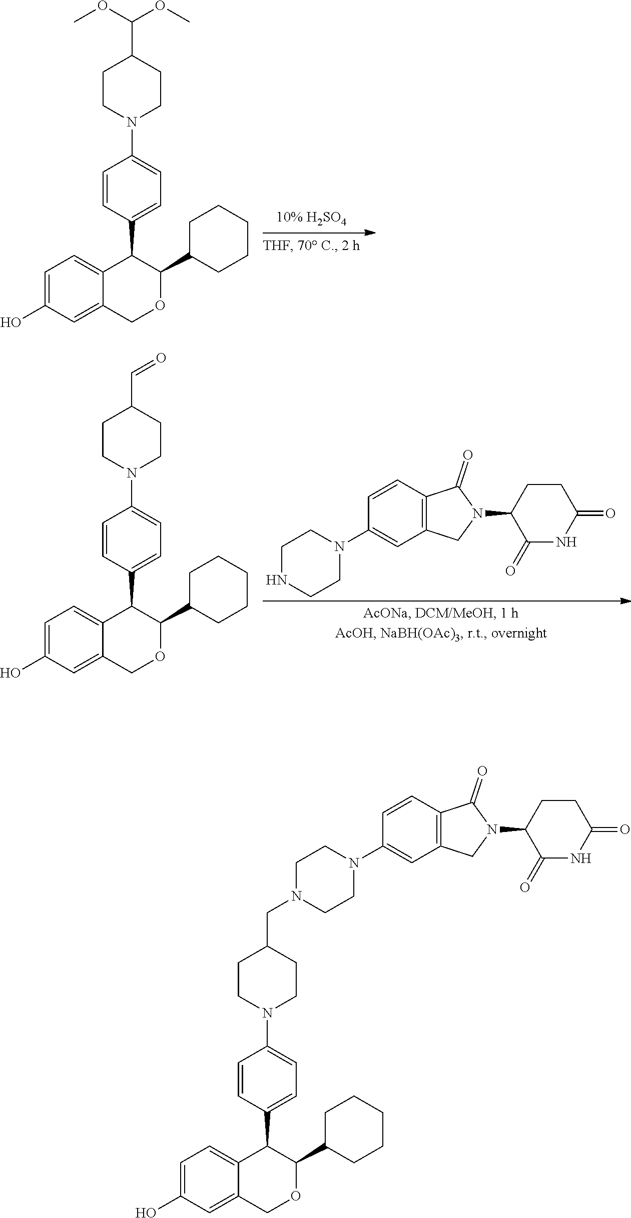

Step 1: A mixture of 2-cyclohexyl-2-hydroxyacetic acid (5.0 g, 1 Eq, 32 mmol), Cesium fluoride (7.2 g, 1.5 Eq, 47 mmol) and Methyl iodide (6.7 g, 1.5 Eq, 47 mmol) in DMF (100 mL) was stirred at 20° C. for 16 h. The reaction was quenched with H₂O (150 mL) and extracted with EtOAc (150 mL×2). The organic layer was washed with brine (100 mL) and dried over Na₂SO₄ and concentrated in vacuo to give methyl 2-cyclohexyl-2-hydroxyacetate (5 g, 90% Yield) as a yellow oil

Step 2: A mixture of methyl 2-cyclohexyl-2-hydroxyacetate (5.00 g, 1 Eq, 29.0 mmol) and 1-(benzyloxy)-3-(bromomethyl)benzene (9.66 g, 1.2 Eq, 34.8 mmol) in THF (80 mL) at 0° C., then Sodium hydride (1.51 g, 1.3 Eq, 37.7 mmol) was added to the mixture at 0° C. and stirred for 5 min, the reaction was warmed to 25° C. and stirred for 12 h. The reaction mixture was added to water (200 mL) at 0° C. and extracted with EtOAc (200 mL×3). The organic layer was washed with brine (50 mL) and dried over Na₂SO₄ and concentrated in vacuo to give a residue. The residue was purified by prep-HPLC (column: Welch Xtimate C18 100*40 mm*3 um; mobile phase: [water (FA)-ACN]; B % 65%-100%, 9 min) to give methyl 2-((3-(benzyloxy)benzyl)oxy)-2-cyclohexylacetate (5.2 g, 46% yield, 93.8% purity) as a yellow oil. LC-MS (ESI+) m/z: 386.0 (M+18)⁺.

Step 3: A mixture of methyl 2-((3-(benzyloxy)benzyl)oxy)-2-cyclohexylacetate (5.2 g, 1 Eq, 13 mmol) and sodium hydroxide (1.6 g, 3 Eq, 40 mmol) in THF (30 mL) and MeOH (30 mL) was stirred at 25° C. for 16 h. The reaction was adjust to pH=5 with 1M HCl. Then the mixture was added water (150 mL) and extracted with ethyl acetate (200×2 mL). The combined organic layers were washed with brine (100 mL), dried over anhydrous Na₂SO₄, filtered and concentrated to dryness in vacuo to give 2-((3-(benzyloxy)benzyl)oxy)-2-cyclohexylacetic acid (5.1 g, 100% Yield, 94.168% Purity) as a yellow oil.

Step 4: A mixture of 2-((3-(benzyloxy)benzyl)oxy)-2-cyclohexylacetic acid (5.1 g, 1 Eq, 14 mmol) in DCM (70 mL) was stirred at 0° C. with N₂, then Trifluoroaceticanhydride (11 g, 4 Eq. 54 mmol) was added to the mixture and the reaction was stirred at 0° C. for 16 h. The mixture was added H₂O (150 mL) and extracted with EtOAc (200 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 7-(benzyloxy)-3-cyclohexylisochroman-4-one (1.4 g, 23% Yield, 74.584% purity) as a yellow solid. LC-MS (ESI+) m/z: 336.9 (M+H)⁺

Step 5: To a solution of 1-(4-bromophenyl)-4-(dimethoxymethyl)piperidine (1.4 g, 2 Eq, 4.4 mmol) in THF (15 mL) was added dropwise n-Butyllithium, 2.5M in hexane (1.4 mL, 2.5 M, 1.6 Eq, 3.5 mmol) at −78° C. with N₂. After addition, the mixture was stirred at this temperature for 1 hour, and then 7-(benzyloxy)-3-cyclohexylisochroman-4-one (1 g, 1 Eq, 2.217 mmol) in THE (5 mL) was added dropwise at −78° C. The resulting mixture was stirred at 25° C. for 16 hour. TLC (PE/EtOAc=5/1, R_{f}=0.4). The reaction mixture was quenched into a solution of saturated ammonium chloride solution followed by extraction with EtOAc (100 mL*2). The combined extracts was dried over anhydrous Na₂SO₄, filtered and concentrated to dryness in vacuo to give 7-(benzyloxy)-3-cyclohexyl-4-(4-(4-(dimethoxymethyl)piperidin-1-yl)phenyl)isochroman-4-ol as a brown oil. The brown oil was purified by flash silica gel chromatography. The desired fractions were collected, and concentrated to dryness in vacuo to give 7-(benzyloxy)-3-cyclohexyl-4-(4-(4-(dimethoxymethyl)piperidin-1-yl)phenyl)isochroman-4-ol (960 mg, 1.3 mmol, 59%, 78% purity) as a yellow solid. LC-MS (ESI+) m/z: 572.3 (M+H)⁺.

Step 6: A mixture of 7-(benzyloxy)-3-cyclohexyl-4-(4-(4-(dimethoxymethyl)piperidin-1-yl)phenyl)isochroman-4-ol (700 mg, 1 Eq, 955 μmol), dihydrogen (2.17 mg, 1 Eq, 1.07 mmol), Pd/C (2 g, 10% Wt, 2 Eq, 2 mmol) in THF (10 mL) and MeOH (10 mL) was degassed and purged with H₂ for 3 times, and then the mixture was stirred at 50° C. for 48 h under H₂ atmosphere (15 psi). LCMS showed 43.4% desired MS. The reaction was filtered and concentrated to dryness in vacuo to give the mixture as a brown oil. The brown oil was purified by Prep. HPLC. Condition: Column: Boston Green ODS 150*30 mm*5 um, A: water (FA), B: CAN, at the beginning: A (50%) and B (50%), at the end: A: (20%) and B (80%), Gradient Time (min) 6; 100% B hold Time (min) 1, Flow Rate (ml/min) 30. The pure fractions were collected and the solvent was evaporated under vacuum. The aqueous layer was lyophilized to dryness to give cis-3-cyclohexyl-4-(4-(4-(dimethoxymethyl)piperidin-1-yl)phenyl)isochroman-7-ol (187 mg, 402 μmol, 37.5%, 100% purity) as a brown oil and trans-3-cyclohexyl-4-(4-(4-(dimethoxymethyl)piperidin-1-yl)phenyl)isochroman-7-ol (48 mg, 99 μmol, 9.2%, 95.8% purity) as a brown oil. 2D NMR confirmed. LC-MS (ESI+) m/z: 466.4 (M+H)⁺.

Step 7: The brown oil was purified by SFC. Condition: CO₂-EtOH (0.1% NH₃H₂O); Column: DAICEL CHIRALPAK AD (250 mm*30 mm, 10 um), Begin B: 45%, End B: 45%, Gradient Time (min): 45, Flow Rate (ml/min): δ 0. The pure fractions were collected and the solvent was evaporated under vacuum. The aqueous layer was lyophilized to dryness to give (3R,4R)-3-cyclohexyl-4-(4-(4-(dimethoxymethyl)piperidin-1-yl)phenyl)isochroman-7-ol (38 mg, 80 μmol, 97.71% purity) as a white solid, (3S,4S)-3-cyclohexyl-4-(4-(4-(dimethoxymethyl)piperidin-1-yl)phenyl)isochroman-7-ol (35 mg, 100% Purity) as a white solid. LC-MS (ESI+) m/z: 466.3 (M+H)⁺.

Step 8: To a solution of (3R,4R)-3-cyclohexyl-4-(4-(4-(dimethoxymethyl)piperidin-1-yl)phenyl)isochroman-7-ol (35 mg, 1 Eq, 73 μmol) in THF (7 mL) was added sulfuric acid (72 mg, 1 Eq, 73 μmol). The mixture was stirred at 70° C. for 3 h. LCMS showed 94.7% desired MS. The mixture was adjusted to pH=8 by progressively adding NaHCO₃. The reaction mixture was quenched by addition 100 mL BtOAc at 20° C., and then diluted with 50 mL H₂O and extracted with EtOAc 50 mL (25 mL×2). The combined organic layers were washed with brine (10 mL), dried over Na₂SO₄. The reaction was filtered and concentrated under reduced pressure to give 1-(4-((3R,4R)-3-cyclohexyl-7-hydroxyisochroman-4-yl)phenyl)piperidine-4-carbaldehyde (64 mg, 94.7% purity, crude) as a brown solid. LC-MS (ESI+) m/z: 438.3 (M+H)⁺.

Step 9: A mixture of 1-(4-((3R,4R)-3-cyclohexyl-7-hydroxyisochroman-4-yl)phenyl)piperidine-4-carbaldehyde (64 mg, 1 Eq, 0.14 mmol), (S)-3-(1-oxo-5-(piperazin-1-yl)isoindolin-2-yl)piperidine-2,6-dione, PhSO₃H salt (72 mg, 1 Eq, 0.14 mmol) and sodium acetate (59 mg, 5 Eq, 0.72 mmol) in DCM (2 mL) and MeOH (2 mL) at 20° C. for 0.5 h, pH=8, then acetic acid (26 mg, 3 Eq, 0.43 mmol) at 20° C. for 1 h, pH=6, then added sodium triacetoxyhydroborate (61 mg, 2 Eq, 0.29 mmol) the mixture, and then the mixture was stirred at 25° C. for 16 h. LCMS showed 41.4% desired MS. The reaction was concentrated to give (S)-3-(5-(4-((1-(4-((3*R,4*R)-3-cyclohexyl-7-hydroxyisochroman-4-yl)phenyl)piperidin-4-yl)methyl)piperazin-1-yl)-1-oxoisoindolin-2-yl)piperidine-2,6-dione as a brown oil. The reaction mixture was diluted with 50 mL H₂O and extracted with EtOAc (25 mL*2). The brown oil was purified by preparative high-performance liquid chromatography. Condition: Column: Welch Xtimate C18 150*30 mm*5 um, A: water (FA), B: CAN, at the beginning: A (73%) and B (27%), at the end: A: (43%) and B (57%), Gradient Time (min) 7; 100% B hold Time (min) 0, Flow Rate (ml/min) 25. The pure fractions were collected and the solvent was evaporated under vacuum. The aqueous layer was lyophilized to dryness to give (S)-3-(5-(4-((1-(4-((3R,4R)-3-cyclohexyl-7-hydroxyisochroman-4-yl)phenyl)piperidin-4-yl)methyl)piperazin-1-yl)-1-oxoisoindolin-2-yl)piperidine-2,6-dione (21 mg, 28 μmol, 20%, 98.76% purity) as a white solid.

LCMS: calc. for C₄₄H₅₃N₅O₅:731.4, found: [M+H]⁺ 732.3.

HPLC: 98.76% purity at 220 nm.

¹H NMR (400 MHZ, DMSO-d₆) δ=10.95 (s, 1H), 9.19 (s, 1H), 8.14 (s, 0.461H), 7.52 (d, J=8.6 Hz, 1H), 7.09-7.04 (m, 4H), 6.75 (dd, J=8.6, 11.3 Hz, 3H), 6.48 (dd, J=2.6, 8.3 Hz, 1H), 6.42 (d, J=2.1 Hz, 1H), 5.04 (dd, J=5.1, 13.2 Hz, 1H), 4.87 (d, J=15.3 Hz, 1H), 4.64 (br d, J=15.3 Hz, 1H), 4.36-4.29 (m, 1H), 4.23-4.16 (m, 1H), 3.74 (d, J=2.4 Hz, 1H), 3.60 (br dd, J=1.8, 4.2 Hz, 2H), 3.29 (br d, J=3.7 Hz, 9H), 2.89 (s, 1H), 2.58 (br d, J=13.8 Hz, 4H), 2.38-2.32 (m, 1H), 2.21 (br d, J=6.6 Hz, 2H), 1.99-1.93 (m, 2H), 1.80 (br d, J=11.0 Hz, 3H), 1.61 (br dd, J=2.4, 6.9 Hz, 4H), 1.24-1.17 (m, 2H), 1.03-0.88 (m, 5H).

### Example 65. Preparation of (I-250) (S)-3-(5-(4-((1-(4-((3S,4S)-3-cyclohexyl-7-hydroxyisochroman-4-yl)phenyl)piperidin-4-yl)methyl)piperazin-1-yl)-1-oxoisoindolin-2-yl)piperidine-2,6-dione

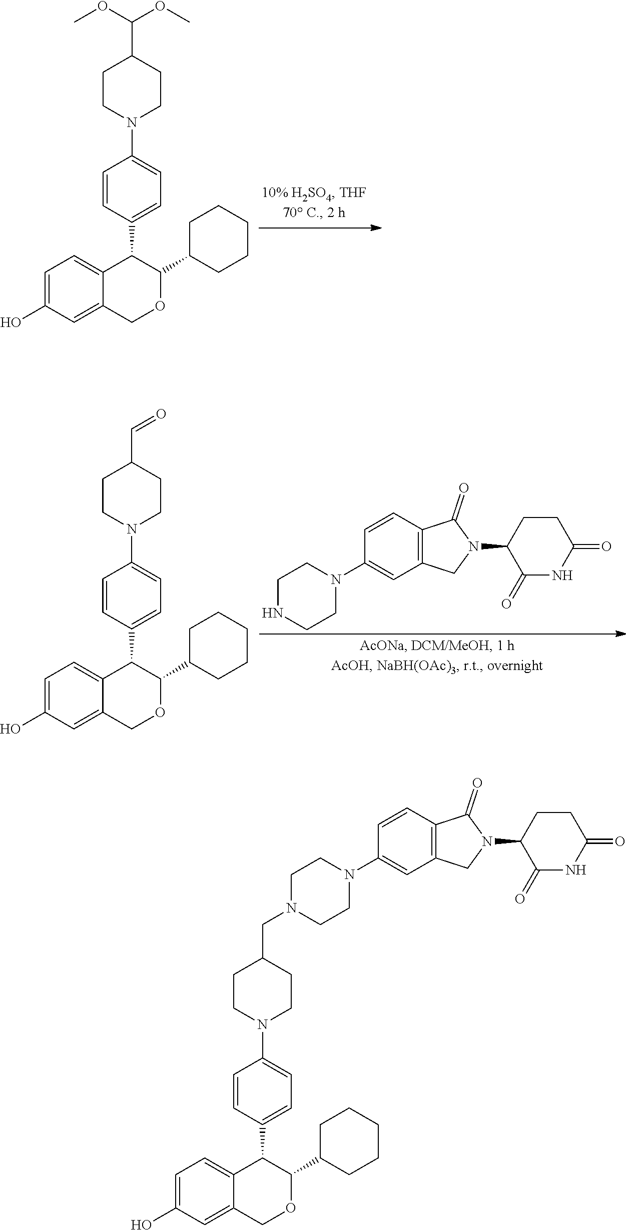

Step 1: To a solution of (3S,4S)-3-cyclohexyl-4-(4-(4-(dimethoxymethyl)piperidin-1-yl)phenyl)isochroman-7-ol (35 mg, 100% Wt, 1 Eq, 75 μmol) in THF (7 mL) was added sulfuric acid (74 mg, 7 mL, 10% Wt, 1 Eq, 75 μmol). The mixture was stirred at 70° C. for 3 hour. LCMS showed 97% desired MS. The mixture was adjusted to pH 8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL, dried over Na₂SO₄. The reaction was filtered and concentrated under reduced pressure to give 1-(4-((3S,4S)-3-cyclohexyl-7-hydroxyisochroman-4-yl)phenyl)piperidine-4-carbaldehyde (50 mg, 0.12 mmol, crude, 97% purity) as a brown solid. LC-MS (ESI+) m/z: 438.3 (M+H)⁺.

Step 2: A mixture of 1-(4-((3S,4S)-3-cyclohexyl-7-hydroxyisochroman-4-yl)phenyl)piperidine-4-carbaldehyde (50 mg, 97% Wt, 1 Eq, 0.12 mmol), (S)-3-(1-oxo-5-(piperazin-1-yl)isoindolin-2-yl)piperidine-2,6-dione, PhSO₃H salt (58 mg, 1 Eq, 0.12 mmol) and sodium acetate (47 mg, 5 Eq, 0.58 mmol) in DCM (2 mL) and MeOH (2 mL) at 20° C. for 0.5 hour, pH=8, then acetic acid (21 mg, 20 μL, 3 Eq, 0.35 mmol) at 20° C. for 1 hour, pH=6, then added sodium triacetoxyhydroborate (49 mg, 2 Eq, 0.23 mmol) the mixture, and then the mixture was stirred at 25° C. for 16 hour. LCMS showed 41.59% desired MS. The reaction was concentrated to give (S)-3-(5-(4-((1-(4-((3S,4S)-3-cyclohexyl-7-hydroxyisochroman-4-yl)phenyl)piperidin-4-yl)methyl)piperazin-1-yl)-1-oxoisoindolin-2-yl)piperidine-2,6-dione as a brown oil. The mixture was added 20 mL water and extracted by EtOAc (50 mL×3). The brown oil was purified by preparative high-performance liquid chromatography. Condition: Column: Welch Xtimate C18 150*30 mm*5 um, A: water (FA), B: CAN, at the beginning: A (73%) and B (27%), at the end: A: (43%) and B (57%), Gradient Time (min) 7; 100% B hold Time (min) 0, Flow Rate (ml/min) 25. The pure fractions were collected and the solvent was evaporated under vacuum. The aqueous layer was lyophilized to dryness to give (S)-3-(5-(4-((1-(4-((3S,4S)-3-cyclohexyl-7-hydroxyisochroman-4-yl)phenyl)piperidin-4-yl)methyl)piperazin-1-yl)-1-oxoisoindolin-2-yl)piperidine-2,6-dione (18.8 mg, 25.1 μmol, 22%, 97.61% purity) as a white solid.

LCMS: calc. C₄₄H₅₃N₅O₅:731.4, found: [M+H]⁺ 732.3.

HPLC: 97.61% purity at 220 nm.

¹H NMR (400 MHZ, DMSO-d₆) δ=10.95 (s, 1H), 9.28-9.10 (m, 1H), 8.16 (s, 0.506H), 7.52 (d, J=8.6 Hz, 1H), 7.11-7.02 (m, 4H), 6.75 (dd, J=8.7, 11.1 Hz, 3H), 6.48 (dd, J=2.6, 8.3 Hz, 1H), 6.42 (d, J=2.3 Hz, 1H), 5.04 (dd, J=5.0, 13.2 Hz, 1H), 4.87 (d, J=15.6 Hz, 1H), 4.64 (br d, J=15.1 Hz, 1H), 4.36-4.29 (m, 1H), 4.22 (s, 1H), 3.74 (br d, J=1.5 Hz, 1H), 3.59 (br d, J=4.8 Hz, 3H), 3.29 (br d, J=3.2 Hz, 8H), 2.88 (br d, J=12.6 Hz, 1H), 2.63-2.53 (m, 4H), 2.40-2.31 (m, 1H), 2.21 (brd, J=6.9 Hz, 2H), 1.99-1.92 (m, 2H), 1.80 (br d, J=11.6 Hz, 3H), 1.68-1.49 (m, 4H), 1.25-1.17 (m, 2H), 1.03-0.85 (m, 5H).

### Example 66. Preparation of (I-248) (S)-3-(5-(4-((1-(4-((3R,4S)-3-cyclohexyl-7-hydroxyisochroman-4-yl)phenyl)piperidin-4-yl)methyl)piperazin-1-yl)-1-oxoisoindolin-2-yl)piperidine-2,6-dione

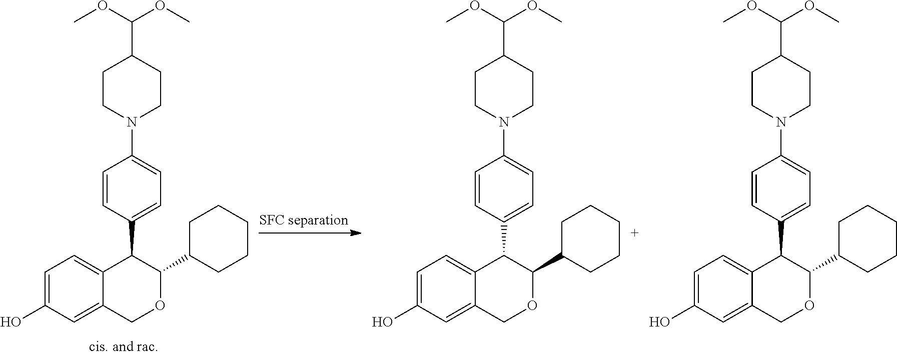 [SMILES: COC(OC)C1CCN(c2ccc([C@@H]3c4ccc(O)cc4CO[C@H]3C3CCCCC3)cc2)CC1 ; COC(OC)C1CCN(c2ccc([C@H]3c4ccc(O)cc4CO[C@@H]3C3CCCCC3)cc2)CC1 ; COC(OC)C1CCN(c2ccc([C@@H]3c4ccc(O)cc4CO[C@H]3C3CCCCC3)cc2)CC1]

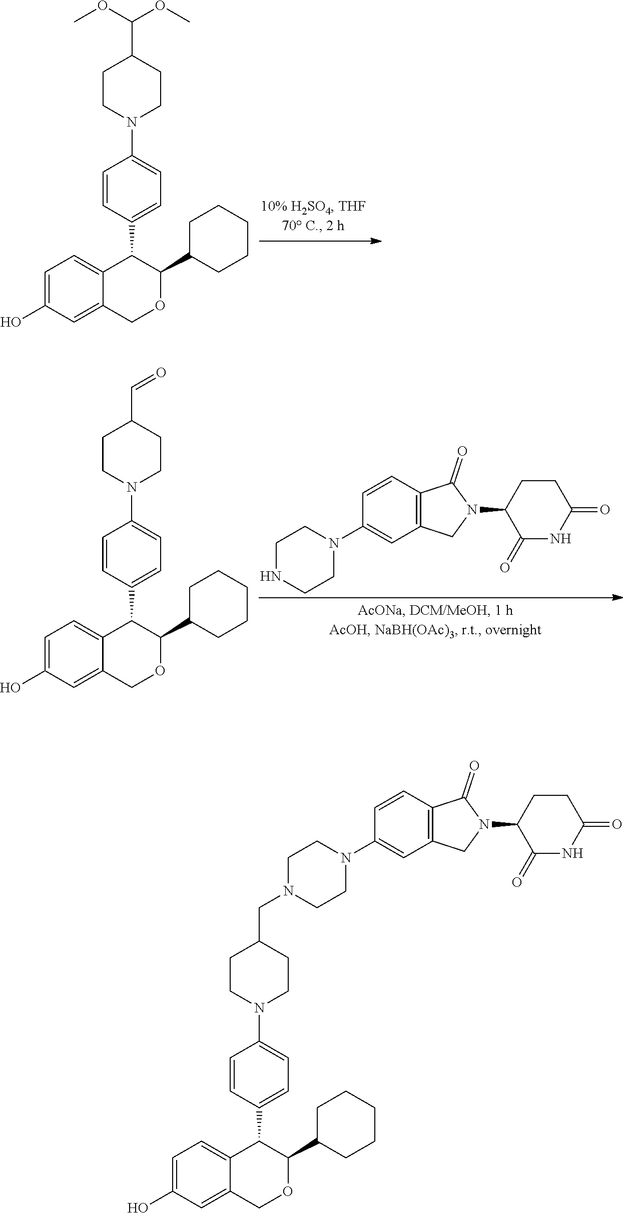

Step 1: The brown oil was purified by SFC. Condition: CO₂-EtOH (0.1% NH₃H₂O); Column: DAICEL CHIRALPAK AD (250 mm*30 mm, 10 um), Begin B: 40%, End B: 40%, FlowRate (ml/min): δ 0, The pure fractions were collected and the solvent was evaporated under vacuum. The aqueous layer was lyophilized to dryness to give (3R,4S)-3-cyclohexyl-4-(4-(4-(dimethoxymethyl)piperidin-1-yl)phenyl)isochroman-7-ol (15 mg, 30 μmol, 19%, 93.7% purity) as a colorless oil, (3S,4R)-3-cyclohexyl-4-(4-(4-(dimethoxymethyl)piperidin-1-yl)phenyl)isochroman-7-ol (15 mg, 30 μmol, 18%, 92.61% purity) as a colorless oil. LC-MS (ESI+) m/z: 466.3 (M+H)⁺.

Step 2: To a solution of (3R,4S)-3-cyclohexyl-4-(4-(4-(dimethoxymethyl)piperidin-1-yl)phenyl)isochroman-7-ol (15 mg, 93.7% Wt, 1 Eq, 30 μmol) in THF (2 mL) was added sulfuric acid (30 mg, 2 mL, 10% Wt, 1 Eq, 30 μmol). The mixture was stirred at 70° C. for 3 hour. LCMS showed 97.97% desired MS. The mixture was adjusted to pH 8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL, dried over Na₂SO₄. The reaction was filtered and concentrated under reduced pressure to give 1-(4-((3R,4S)-3-cyclohexyl-7-hydroxyisochroman-4-yl)phenyl)piperidine-4-carbaldehyde (30 mg, 70 μmol, crude, 97.97% purity) as a brown oil. LC-MS (ESI+) m/z: 438.0 (M+H)⁺.

Step 3: A mixture of 1-(4-((3R,4S)-3-cyclohexyl-7-hydroxyisochroman-4-yl)phenyl)piperidine-4-carbaldehyde (30 mg, 1 Eq, 70 μmol), (S)-3-(1-oxo-5-(piperazin-1-yl)isoindolin-2-yl)piperidine-2,6-dione, PhSO3H salt (35 mg, 1 Eq, 70 μmol) and sodium acetate (29 mg, 5 Eq, 0.35 mmol) in DCM (2 mL) and MeOH (2 mL) at 20° C. for 0.5 hour, pH=8, then acetic acid (13 mg, 12 μL, 3 Eq, 0.21 mmol) at 20° C. for 1 hour, pH=6, then added sodium triacetoxyhydroborate (30 mg, 2 Eq, 0.14 mmol) the mixture, and then the mixture was stirred at 25° C. for 16 hour. The reaction was concentrated to give (S)-3-(5-(4-((1-(4-((3R,4S)-3-cyclohexyl-7-hydroxyisochroman-4-yl)phenyl)piperidin-4-yl)methyl)piperazin-1-yl)-1-oxoisoindolin-2-yl)piperidine-2,6-dione as a brown oil. The reaction mixture was diluted with 50 mL H₂O and extracted with EtOAc (25 mL*2). The brown oil was purified by preparative high-performance liquid chromatography. Condition: Column: Welch Xtimate C18 150*30 mm*5 um, A: water (FA), B: CAN, at the beginning: A (79%) and B (21%), at the end: A: (49%) and B (51%), Gradient Time (min) 7; 100% B hold Time (min) 0.5, Flow Rate (ml/min) 25. The pure fractions were collected and the solvent was evaporated under vacuum. The aqueous layer was lyophilized to dryness to give (S)-3-(5-(4-((1-(4-((3R,4S)-3-cyclohexyl-7-hydroxyisochroman-4-yl)phenyl)piperidin-4-yl)methyl)piperazin-1-yl)-1-oxoisoindolin-2-yl)piperidine-2,6-dione (8.1 mg, 11 μmol, 16%, 98.69% purity) as a white solid.

LCMS: calc. C₄₄H₅₃N₅O₅: 731.94, found: [M+H]⁺ 732.4.

HPLC: 98.69% purity at 220 nm.

¹H NMR (400 MHz, DMSO-d₆) δ=10.95 (s, 1H), 9.17 (s, 1H), 8.13 (s, 0.275H), 7.52 (d, J=8.6 Hz, 1H), 7.09-7.04 (m, 2H), 6.97-6.92 (m, 2H), 6.89-6.83 (m, 2H), 6.47-6.39 (m, 3H), 5.05 (dd, J=4.7, 13.1 Hz, 1H), 4.72 (s, 2H), 4.33 (d, J=16.9 Hz, 1H), 4.24-4.16 (m, 1H), 3.90 (br d, J=10.3 Hz, 1H), 3.70-3.61 (m, 2H), 3.46 (br d, J=2.0 Hz, 1H), 2.61 (br d, J=10.7 Hz, 5H), 2.43-2.36 (m, 3H), 2.24 (br dd, J=1.6, 4.9 Hz, 3H), 2.02-1.90 (m, 2H), 1.83 (br d, J=11.3 Hz, 4H), 1.73-1.53 (m, 5H), 1.42-1.35 (m, 2H), 1.29-1.12 (m, 5H), 1.09-1.00 (m, 3H).

### Example 67. Preparation of (I-249) (S)-3-(5-(4-((1-(4-((3S,4R)-3-cyclohexyl-7-hydroxyisochroman-4-yl)phenyl)piperidin-4-yl)methyl)piperazin-1-yl)-1-oxoisoindolin-2-yl)piperidine-2,6-dione

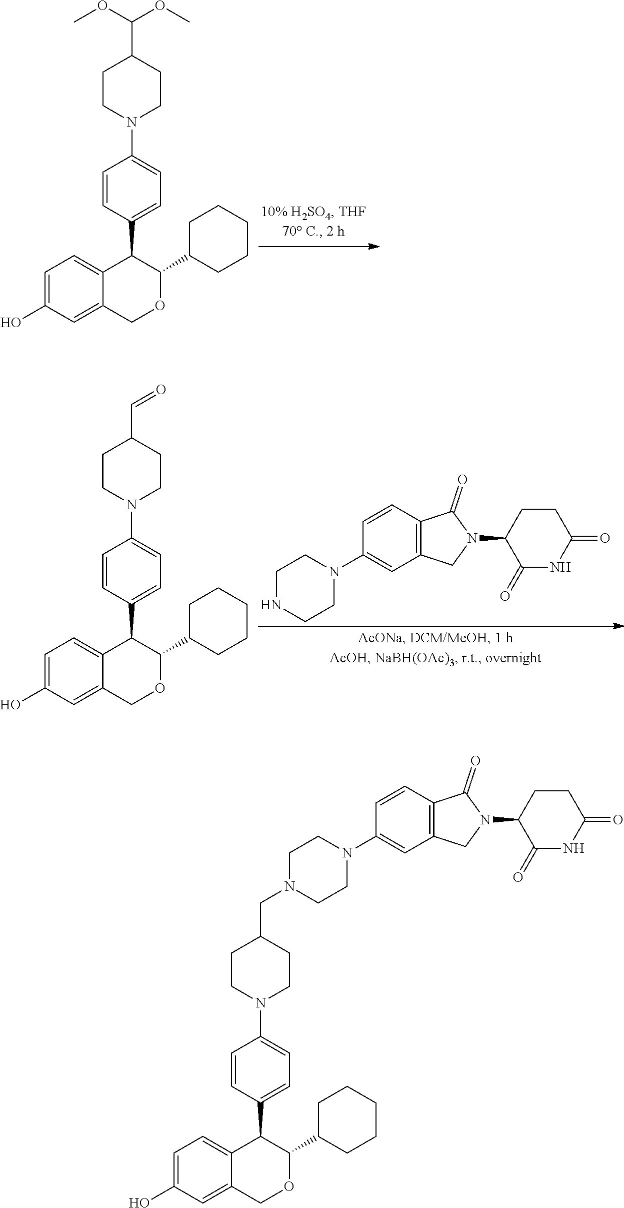

Step 1: To a solution of (3S,4R)-3-cyclohexyl-4-(4-(4-(dimethoxymethyl)piperidin-1-yl)phenyl)isochroman-7-ol (15 mg, 92.61% Wt, 1 Eq, 30 μmol) in THF (2 mL) was added sulfuric acid (29 mg, 2 mL, 10% Wt, 1 Eq, 30 μmol). The mixture was stirred at 70° C. for 3 hour. LCMS showed 98.01% desired MS. The mixture was adjusted to pH 8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL, dried over Na₂SO₄. The reaction was filtered and concentrated under reduced pressure to give 1-(4-((3S,4R)-3-cyclohexyl-7-hydroxyisochroman-4-yl)phenyl)piperidine-4-carbaldehyde (30 mg, 70 μmol, crude, 98.01% purity) as a brown oil. LC-MS (ESI+) m/z: 438.0 (M+H)⁺.

Step 2: A mixture of 1-(4-((3S,4R)-3-cyclohexyl-7-hydroxyisochroman-4-yl)phenyl)piperidine-4-carbaldehyde (30 mg, 1 Eq, 70 μmol), (S)-3-(1-oxo-5-(piperazin-1-yl)isoindolin-2-yl)piperidine-2,6-dione, Tosylic acid (35 mg, 1 Eq, 70 μmol) and sodium acetate (29 mg, 5 Eq, 0.35 mmol) in DCM (2 mL) and MeOH (2 mL) at 20° C. for 0.5 hour, pH=8, then acetic acid (13 mg, 12 μL, 3 Eq, 0.21 mmol) at 20° C. for 1 hour, pH-6, then added sodium triacetoxyhydroborate (30 mg, 2 Eq, 0.14 mmol) the mixture, and then the mixture was stirred at 25° C. for 16 hour. LCMS showed 41.4% desired MS. The reaction was concentrated to give (S)-3-(5-(4-((1-(4-((3S,4R)-3-cyclohexyl-7-hydroxyisochroman-4-yl)phenyl)piperidin-4-yl)methyl)piperazin-1-yl)-1-oxoisoindolin-2-yl)piperidine-2,6-dione. The reaction mixture was diluted with 50 mL H₂O and extracted with EtOAc (25 mL*2). The brown oil was purified by preparative high-performance liquid chromatography. Condition: Column: Welch Xtimate C18 150*30 mm*5 um, A: water (FA), B: CAN, at the beginning: A (79%) and B (21%), at the end: A: (49%) and B (51%), Gradient Time (min) 7; 100% B hold Time (min) 0.5, Flow Rate (ml/min) 25. The pure fractions were collected and the solvent was evaporated under vacuum. The aqueous layer was lyophilized to dryness to give (S)-3-(5-(4-((1-(4-((3S,4R)-3-cyclohexyl-7-hydroxyisochroman-4-yl)phenyl)piperidin-4-yl)methyl)piperazin-1-yl)-1-oxoisoindolin-2-yl)piperidine-2,6-dione (5.8 mg, 7.9 μmol, 11%, 100% Purity) as a white solid.

LCMS: calc. C₄₄H₅₁N₅O₅: 731.9, found: [M+H]⁺ 732.4.

HPLC: 100% purity at 220 nm.

¹H NMR (400 MHZ, DMSO-d₆) δ=10.95 (s, 1H), 9.18 (br d, J=2.6 Hz, 1H), 8.18 (s, 0.286H), 7.52 (br d, J=8.7 Hz, 1H), 7.10-7.04 (m, 2H), 6.98-6.91 (m, 2H), 6.86 (br d, J=8.5 Hz, 2H), 6.48-6.37 (m, 3H), 5.10-5.01 (m, 1H), 4.72 (s, 2H), 4.38-4.28 (m, 1H), 4.23 (s, 1H), 3.90 (br d. J=9.8 Hz, 1H), 3.69-3.61 (m, 2H), 3.45 (br d, J=11.3 Hz, 2H), 2.98-2.80 (m, 2H), 2.61 (br d, J=10.8 Hz, 5H), 2.39 (br s, 3H), 2.23 (br d, J=6.9 Hz, 2H), 1.99-1.93 (m, 1H), 1.87-1.80 (m, 3H), 1.75-1.51 (m, 5H), 1.42-1.34 (m, 2H), 1.30-1.12 (m, 5H), 1.04 (br d, J=1.1 Hz, 3H).

### Example 68. Preparation of (I-418) (S)-3-(5-(4-((1-(4-((3R,4R)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione

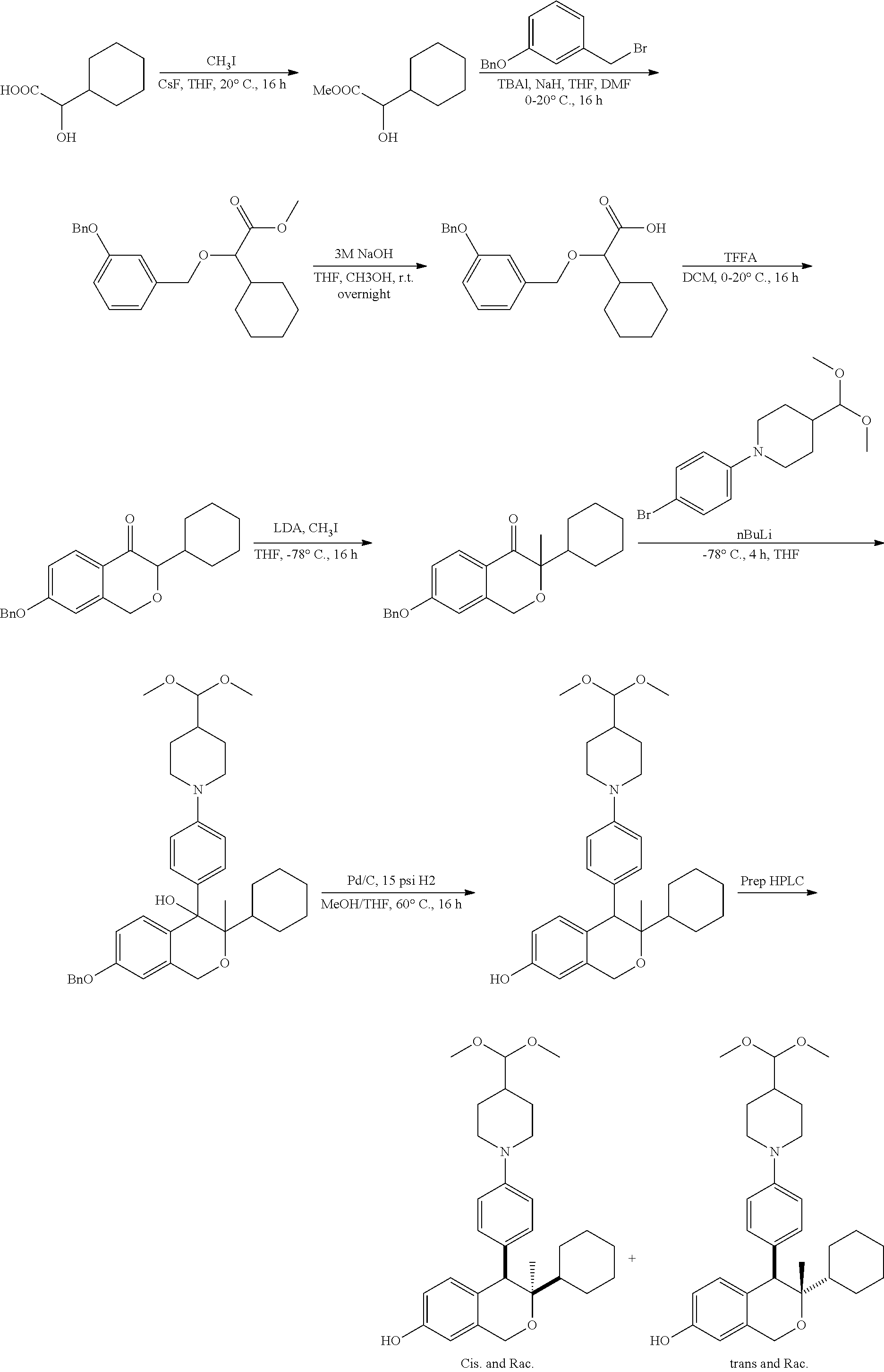

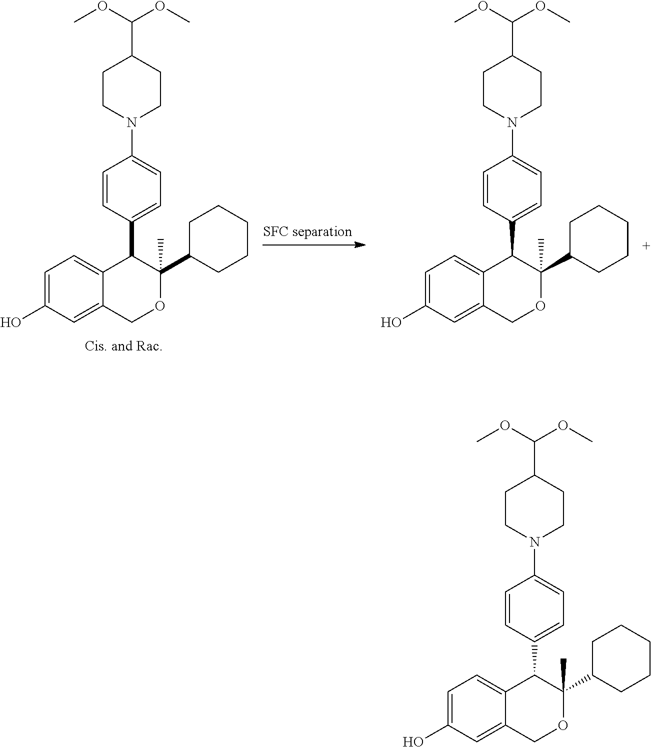 [SMILES: COC(OC)C1CCN(c2ccc([C@@H]3c4ccc(O)cc4CO[C@]3(C)C3CCCCC3)cc2)CC1 ; COC(OC)C1CCN(c2ccc([C@@H]3c4ccc(O)cc4CO[C@]3(C)C3CCCCC3)cc2)CC1 ; COC(OC)C1CCN(c2ccc([C@H]3c4ccc(O)cc4CO[C@@]3(C)C3CCCCC3)cc2)CC1]

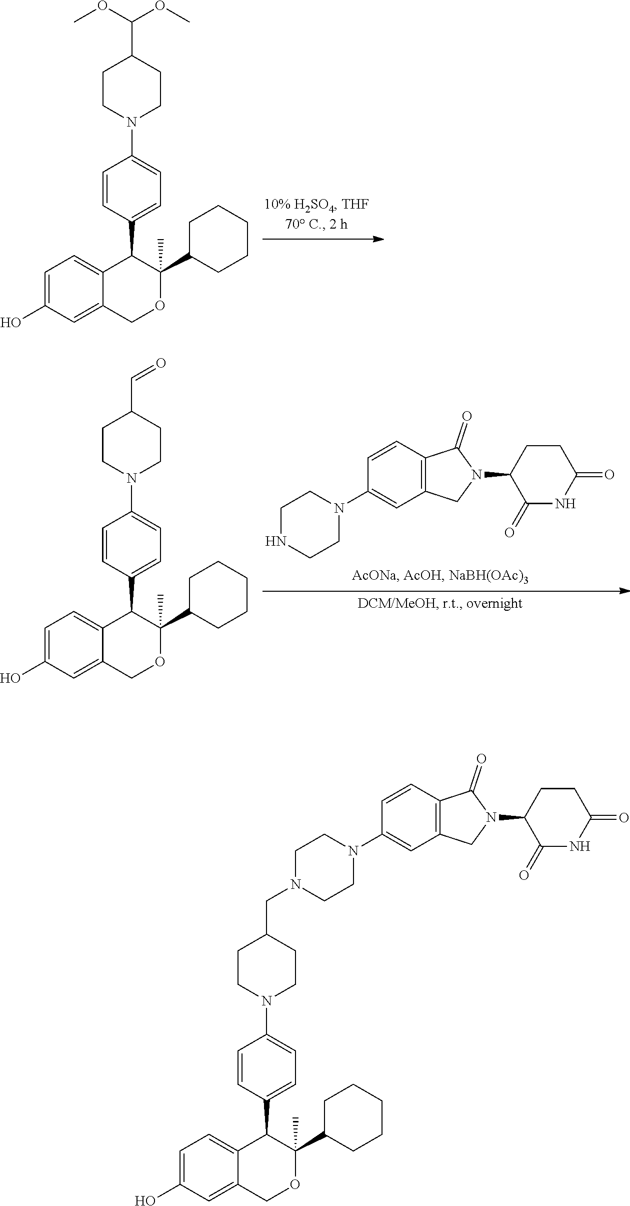 [SMILES: COC(OC)C1CCN(c2ccc([C@@H]3c4ccc(O)cc4CO[C@]3(C)C3CCCCC3)cc2)CC1 ; C[C@]1(C2CCCCC2)OCc2cc(O)ccc2[C@H]1c1ccc(N2CCC(C=O)CC2)cc1 ; O=C1CC[C@H](N2Cc3cc(N4CCNCC4)ccc3C2=O)C(=O)N1 ; C[C@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1]

Step 1: A mixture of 2-cyclohexyl-2-hydroxyacetic acid (10.0 g, 1 Eq, 63.2 mmol), Cesium fluoride (14.4 g, 3.50 mL, 1.5 Eq, 94.8 mmol) and methyl iodide (13.5 g, 6.13 mL, 1.5 Eq, 94.8 mmol) in DMF (80 mL) was stirred at 20° C. for 16 hour. TLC (PE:EtOAc=5:1, R_{f}=0.5) showed a new spot was detected. The mixture was added H₂O (300 mL) and extracted with EtOAc (200 mL*2). The combined extracts was dried over anhydrous Na₂SO₄, filtered and concentrated to dryness in vacuo to give methyl 2-cyclohexyl-2-hydroxyacetate (10.52 g, 61.08 mmol, 96.6%) as a brown oil. NMR confirmed.

Step 2: A mixture of methyl 2-cyclohexyl-2-hydroxyacetate (10.52 g, 1 Eq, 61.08 mmol) and 1-(benzyloxy)-3-(bromomethyl)benzene (20.32 g, 1.2 Eq, 73.30 mmol) in THF (200 mL) at 0° C., then Sodium hydride (3.176 g, 2.6 mL, 60% Wt, 1.3 Eq, 79.41 mmol) was added to the mixture at 0° C. and stirred for 5 min, the reaction was warmed to 25° C. and stirred for 12 hour. The reaction mixture was added to water (300 mL) at 0° C. and extracted with ethyl acetate (300 mL*3). The combined organic layers were washed with brine, dried over anhydrous sodium sulfate, filtered and concentrated under reduced pressure to give a residue. The residue was purified by Prep.HPLC. Column: Xtimate C18 150*40 mm*5 um, Condition: water (FA)-MeOH, at the beginning: A (35%) and B (65%), at the end: A (0%) and B (100%), Gradient Time (min) 9, 100% B hold Time (min) 2, Flow Rate (ml/min) 30. The pure fractions were collected and the solvent was evaporated under vacuum. The aqueous layer was lyophilized to dryness to give methyl 2-((3-(benzyloxy)benzyl)oxy)-2-cyclohexylacetate (8.79 g, 22.4 mmol, 36.6%, 93.8% purity) as a yellow oil. LCMS and NMR confirmed. LC-MS (ESI⁺) m/z: 391.1 (M+23)⁺.

Step 3: A mixture of methyl 2-((3-(benzyloxy)benzyl)oxy)-2-cyclohexylacetate (8.79 g, 93.8% Wt, 1 Eq, 22.4 mmol) and sodium hydroxide (2.69 g, 22.4 mL, 3 molar, 3 Eq, 67.1 mmol) in THF (30 mL) and MeOH (30 mL) was stirred at 25° C. for 16 hour. The reaction was adjust to pH=˜5 with 1M HCl (40 mL). Then the mixture was added water (200 mL) and extracted with ethyl acetate (3×150 mL). The combined organic layers were washed with brine (100 mL), dried over anhydrous Na₂SO₄, filtered and concentrated to dryness in vacuo to give 2-((3-(benzyloxy)benzyl)oxy)-2-cyclohexylacetic acid (8.836 g, 21 mmol, 94%, 84% purity) as a yellow oil. NMR confirmed. LC-MS (ESI⁺) m/z: 371.9 (M+23)⁺.

Step 4: A mixture of 2-((3-(benzyloxy)benzyl)oxy)-2-cyclohexylacetic acid (8.836 g, 84% Wt, 1 Eq, 20.94 mmol) in DCM (100 mL) was stirred at 0° C. with N₂, then Trifluoroaceticanhydride (17.59 g, 11.70 mL, 4 Eq, 83.76 mmol) was added to the mixture and the reaction was stirred at 0° C. for 16 hour. TLC (PE/EtOAc=10/1, R_{f}=0.4) showed a new spot was detected. The mixture was added H₂O (150 mL) and extracted with EtOAc (200 mL*2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 90/10). The pure fractions were collected and concentrated to dryness in vacuo to give 7-(benzyloxy)-3-cyclohexylisochroman-4-one (1.35 g, 3.96 mmol, 18.9%, 98.75% Purity) as a yellow solid. The yellow solid was purified by Prep.HPLC. Condition: Column: Xtimate C18 150*40 mm*5 um, A: water (FA), B: CAN, at the beginning: A (30%) and B (70%), at the end: A: (0%) and B (100%), Gradient Time (min) 9; 100% B hold Time (min) 5, Flow Rate (ml/min) 30. The pure fractions were collected and the solvent was evaporated under vacuum. The aqueous layer was lyophilized to dryness to give 7-(benzyloxy)-3-cyclohexylisochroman-4-one (1.35 g, 3.96 mmol, 18.9%, 98.75% Purity) as a yellow solid. LC-MS (ESI+) m/z: 337.1 (M+H)⁺

Step 5: To a solution of 7-(benzyloxy)-3-cyclohexylisochroman-4-one (1.12 g, 1 Eq, 3.33 mmol) in THF (15 mL) was added LDA (713 mg, 3.33 mL, 2 molar, 2 Eq, 6.66 mmol) at −70° C. under N₂. The reaction mixture was stirred at −70° C. for 1 hour. Then methyl iodide (1.42 g, 646 μL, 3 Eq. 9.99 mmol) was added to the mixture dropwise at −70° C. The reaction mixture was warmed to 25° C. and stirred for 16 hour. TLC (PE/EtOAc=10/1, R_{f}=0.4) showed two spots. The reaction mixture was quenched into a solution of saturated ammonium chloride solution followed by extraction with EtOAc (100 mL*2). The combined extracts was dried over anhydrous Na₂SO₄, filtered and concentrated to dryness in vacuo to give mixture as a yellow solid. The yellow solid was purified by flash silica gel chromatography (from PE/EtOAc=1/0 to 10/1). The desired fractions were collected, and concentrated to dryness in vacuo to give 7-(benzyloxy)-3-cyclohexyl-3-methylisochroman-4-one (400 mg, 1.13 mmol, 34.0%, 99.1% purity) as a colorless oil. The colorless oil was purified by preparative high-performance liquid chromatography. Condition: Column: Boston Green ODS 150*30 mm*5 um, A: water (FA), B: CAN, at the beginning: A (15%) and B (85%), at the end: A: (0%) and B (100%), Gradient Time (min) 6; 100% B hold Time (min) 1, Flow Rate (ml/min) 30. The pure fractions were collected and the solvent was evaporated under vacuum. The aqueous layer was lyophilized to dryness to give 7-(benzyloxy)-3-cyclohexyl-3-methylisochroman-4-one (400 mg, 1.13 mmol, 34.0%, 99.1% Purity) as a white solid. NMR confirmed. LC-MS (ESI+) m/z: 351.1 (M+H)⁺.

Step 6: To a solution of 1-(4-bromophenyl)-4-(dimethoxymethyl)piperidine (880 mg, 3 Eq, 2.80 mmol) in THF (20 mL) was added dropwise n-Butyllithium in hexane (149 mg, 933 μL, 2.5 M, 2.5 Eq, 2.33 mmol) at −78° C. with N₂. After addition, the mixture was stirred at this temperature for 1 hour, and then 7-(benzyloxy)-3-cyclohexyl-3-methylisochroman-4-one (330 mg, 1 Eq, 933 μmol) in THF (5 mL) was added dropwise at −78° C. The resulting mixture was stirred at 25° C. for 16 hour. TLC (PE/EtOAc=5/1, R_{f}=0.4) showed three spots. The reaction mixture was quenched into a solution of saturated ammonium chloride solution followed by extraction with EtOAc (100 mL*3). The combined extracts was dried over anhydrous Na₂SO₄, filtered and concentrated to dryness in vacuo to give mixture as a brown oil. The brown oil was purified by flash silica gel chromatography (from PE/EtOAc=1/0 to 25/1). The desired fractions were collected, and concentrated to dryness in vacuo to give 7-(benzyloxy)-3-cyclohexyl-4-(4-(4-(dimethoxymethyl)piperidin-1-yl)phenyl)-3-methylisochroman-4-ol (544 mg, 881 μmol, 94.4%), 94.87% purity) as a yellow oil. LC-MS (ESI+) m/z: 586.3 (M+H)⁺.

Step 7: A mixture of 7-(benzyloxy)-3-cyclohexyl-4-(4-(4-(dimethoxymethyl)piperidin-1-yl)phenyl)-3-methylisochroman-4-ol (524 mg, 94.87% Wt, 1 Eq, 849 μmol), dihydrogen (1.71 mg, 1 Eq, 849 μmol), Pd/C (780 mg, 10% Wt, 0.864 Eq, 733 μmol) in THE (10 mL) and MeOH (10 mL) was degassed and purged with H₂ for 3 times, and then the mixture was stirred at 55° C. for 16 hour under H₂ atmosphere (15 psi). The mixture was filtered and the filter cake was washed by EtOAc (100 mL*3). The brown oil was purified by preparative high-performance liquid chromatography. Condition: water(NH₃H₂O+NH₄HCO₃)-CAN, Column: Boston Prime C18 150*30 mm*5 um, at the beginning: A (75%) and B (70%), at the end: A: (45%) and B (100%)

Gradient Time (min) 7; 100% B hold Time (min) 2, Flow Rate (ml/min) 25. The pure fractions were collected and the solvent was evaporated under vacuum. The reaction was filtered and concentrated to dryness in vacuo to give cis-3-cyclohexyl-4-(4-(4-(dimethoxymethyl)piperidin-1-yl)phenyl)-3-methylisochroman-7-ol (110 mg, 108 μmol, 12.7%, 94.04% purity) as a white solid and trans-3-cyclohexyl-4-(4-(4-(dimethoxymethyl)piperidin-1-yl)phenyl)-3-methylisochroman-7-ol (243 mg, 253 μmol, 29.8%, 100% purity) as a white solid. NMR confirmed. 2D NMR confirmed. LC-MS (ESI+) m/z: 480.2 (M+H)⁺

Step 8: The white solid was purified by SFC. Condition: CO₂-EtOH (0.1% NH₃H₂O); Column: DAICEL CHIRALPAK IG (250 mm*30 mm, 10 um), Begin B: 45%, End B: 45%, FlowRate (ml/min): δ 0. The pure fractions were collected and the solvent was evaporated under vacuum. The aqueous layer was lyophilized to dryness to give (3R,4R)-3-cyclohexyl-4-(4-(4-(dimethoxymethyl)piperidin-1-yl)phenyl)-3-methylisochroman-7-ol (66 mg, 0.14 mmol, 27%, 100% purity) as a white solid and (3S,4S)-3-cyclohexyl-4-(4-(4-(dimethoxymethyl)piperidin-1-yl)phenyl)-3-methylisochroman-7-ol (74 mg, 0.15 mmol, 30%, 100% purity) as a white solid. LC-MS (ESI+) m/z: 480.2 (M+H)⁺.

Step 9: To a solution of (3R,4R)-3-cyclohexyl-4-(4-(4-(dimethoxymethyl)piperidin-1-yl)phenyl)-3-methylisochroman-7-ol (66 mg, 1 Eq, 0.14 mmol) in THF (7 mL) was added sulfuric acid (0.13 g, 7 mL, 10% Wt, 1 Eq. 0.14 mmol). The mixture was stirred at 70° C. for 2 hour. The mixture was adjusted to pH=8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL, dried over Na₂SO₄. The reaction was filtered and concentrated under reduced pressure to give 1-(4-((3R,4R)-3-cyclohexyl-7-hydroxy-3-methylisochroman-4-yl)phenyl)piperidine-4-carbaldehyde (70 mg, 0.16 mmol, 110%, 97.34% purity) as a yellow oil. LC-MS (ESI+) m/z: 452.1 (M+18)⁺.

Step 10: A mixture of 1-(4-((3R,4R)-3-cyclohexyl-7-hydroxy-3-methylisochroman-4-yl)phenyl)piperidine-4-carbaldehyde (70 mg, 97.34% Wt, 1 Eq, 0.16 mmol), (S)-3-(1-oxo-5-(piperazin-1-yl)isoindolin-2-yl)piperidine-2,6-dione PhSO₃H salt (52 mg, 1 Eq, 0.16 mmol) and sodium acetate (64 mg, 5 Eq, 0.79 mmol) in DCM (2 mL) and MeOH (2 mL) at 20° C. for 0.5 hour, pH=8, then acetic acid (28 mg, 27 μL, 3 Eq, 0.47 mmol) at 20° C. for 1 hour, pH=6, then added sodium triacetoxyborohydride (67 mg, 2 Eq, 0.31 mmol) the mixture, and then the mixture was stirred at 25° C. for 16 hour. The reaction was quenched with water (50 mL) and extracted with ethyl acetate (50 mL*2). The reaction was concentrated to give (S)-3-(5-(4-((1-(4-((3R,4R)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione as a colorless oil. The colorless oil was purified by preparative high-performance liquid chromatography. Condition: Column: Welch Xtimate C18 150*30 mm*5 um, A: water (FA), B: CAN, at the beginning: A (87%) and B (13%), at the end: A: (57%) and B (43%), Gradient Time (min) 7; 100% B hold Time (min) 3.5, Flow Rate (ml/min) 25. The pure fractions were collected, and the solvent was evaporated under vacuum. The aqueous layer was lyophilized to dryness to give (S)-3-(5-(4-((1-(4-((3R,4R)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione (36.8 mg, 47.5 μmol, 30%, 96.26% purity) as a white solid.

LCMS: calc. for C₄₄H₅₅N₅O₅: 745.42, found: [M+H]⁺ 746.3.

HPLC: 96.26% purity at 220 nm.

¹H NMR (400 MHz, DMSO-d₆)>=10.95 (s, 1H), 9.14 (br s, 1H), 8.15 (s, 0.321H), 7.51 (d, J==8.6 Hz, 1H), 7.10-7.01 (m, 4H), 6.74 (dd, J=6.2, 8.3 Hz, 3H), 6.49-6.41 (m, 2H), 5.09-5.00 (m, 1H), 4.85-4.78 (m, 1H), 4.72-4.64 (m, 1H), 4.30 (s, 1H), 4.24-4.15 (m, 1H), 3.55 (s, 3H), 3.28 (br s, 5H), 2.94-2.85 (m, 1H), 2.55 (br d, J=6.1 Hz, 6H), 2.36 (br dd, J=4.8, 13.2 Hz, 1H), 2.21 (br s, 2H), 1.98-1.93 (m, 1H), 1.86 (br s, 1H), 1.79 (br d, J=11.0 Hz, 3H), 1.69-1.61 (m, 2H), 1.59-1.49 (m, 2H), 1.24-1.16 (m, 2H), 1.04 (s, 5H), 0.91 (br d, J=9.9 Hz, 3H), 0.72-0.62 (m, 1H).

### Example 69. Preparation of (I-256) (S)-3-(5-(4-((1-(4-((3S,4S)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione

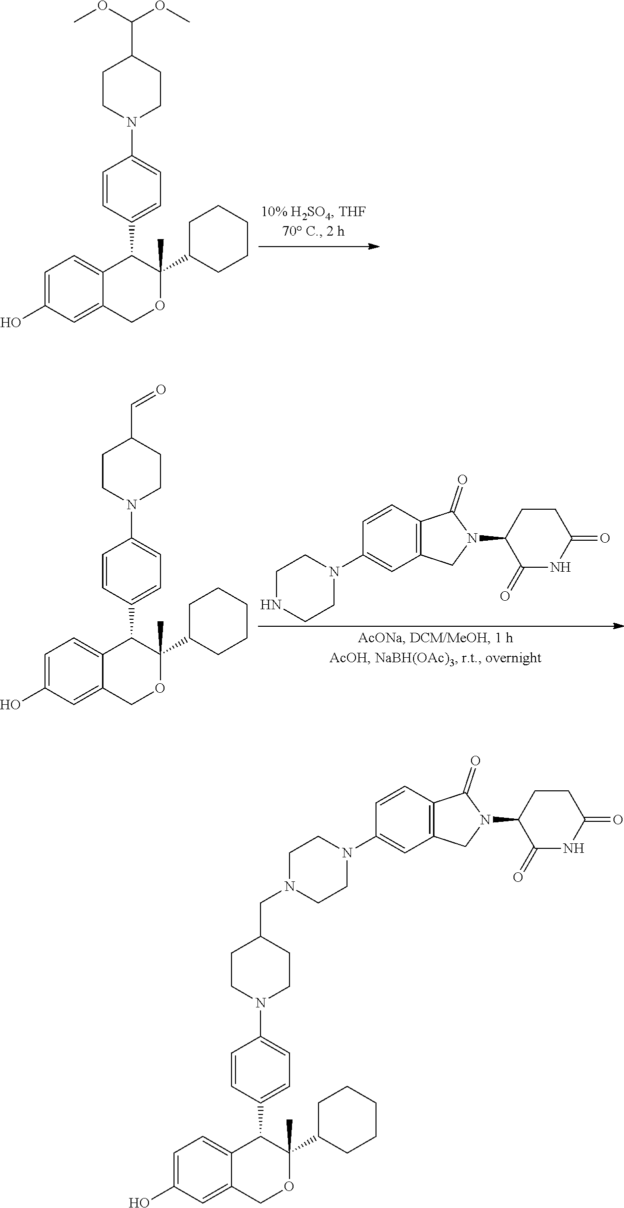

Step 1: To a solution of (3S,4S)-3-cyclohexyl-4-(4-(4-(dimethoxymethyl)piperidin-1-yl)phenyl)-3-methylisochroman-7-ol (74 mg, 100% Wt, 1 Eq, 0.15 mmol) in THF (7 mL) was added sulfuric acid (0.15 g, 7 mL, 10% Wt, 1 Eq, 0.15 mmol). The mixture was stirred at 70° C. for 2 hours. The mixture was adjusted to pH=8-9 by progressively adding NaHCO₃. The reaction mixture was quenched by addition 100 mL EtOAc at 20° C., and then diluted with H₂O 50 mL and extracted with EtOAc 50 mL (25 mL*2). The combined organic layers were washed with, dried over Na₂SO₄. The reaction was filtered and concentrated under reduced pressure to give 1-(4-((3S,4S)-3-cyclohexyl-7-hydroxy-3-methylisochroman-4-yl)phenyl)piperidine-4-carbaldehyde (80 mg, 0.18 mmol, 120%, 98.36% purity) as a brown oil. LC-MS (ESI+) m/z: 452.1 (M+18)⁺.

Step 2: A mixture of 1-(4-((3S,4S)-3-cyclohexyl-7-hydroxy-3-methylisochroman-4-yl)phenyl)piperidine-4-carbaldehyde (80 mg, 98.36% Wt, 1 Eq, 0.18 mmol), (S)-3-(1-oxo-5-(piperazin-1-yl)isoindolin-2-yl)piperidine-2,6-dione PhSO₃H salt (60 mg, 1 Eq, 0.18 mmol) and sodium acetate (74 mg, 5 Eq, 0.91 mmol) in DCM (2 mL) and MeOH (2 mL) at 20° C. for 0.5 hr, pH=8, then acetic acid (33 mg, 31 μL, 3 Eq, 0.54 mmol) at 20° C. for 1 hr, pH=6, then added sodium triacetoxyborohydride (77 mg, 2 Eq, 0.36 mmol) the mixture, and then the mixture was stirred at 25° C. for 16 hour. LCMS showed 77.3% desired MS. The reaction was quenched with water (50 mL) and extracted with ethyl acetate (50 mL*2). The reaction was concentrated to give (S)-3-(5-(4-((1-(4-((3S,4S)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione as a colorless oil. The colorless oil was purified by preparative high-performance liquid chromatography. Condition: Column: Boston Green ODS 150*30 mm*5 um A: water (FA) B: CAN at the beginning: A (75%) and B (25%) at the end: A: (45%) and B (55%) Gradient Time (min) 6; 100% B hold Time (min) 1, Flow Rate (ml/min) 30. The pure fractions were collected, and the solvent was evaporated under vacuum. The aqueous layer was lyophilized to dryness to give (S)-3-(5-(4-((1-(4-((3S,4S)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione (49.5 mg, 65.3 μmol, 36%, 98.48% purity) as a white solid.

LCMS: calc. for C₄₄H₅₅N₅O₅:745.42, found: [M+H]⁺ 746.3.

HPLC: 98.48% purity at 220 nm.

¹H NMR (400 MHZ, DMSO-d₆) δ=10.95 (s, 1H), 9.14 (s, 1H), 8.14 (s, 0.387H), 7.51 (d, J=8.5 Hz, 1H), 7.09-7.02 (m, 4H), 6.74 (dd, J=6.1, 8.2 Hz, 3H), 6.49-6.40 (m, 2H), 5.05 (dd, J=4.9, 13.4 Hz, 1H), 4.86-4.77 (m, 1H), 4.68 (br d, J=16.3 Hz, 1H), 4.37-4.28 (m, 1H), 4.24-4.16 (m, 1H), 3.62-3.54 (m, 3H), 3.28 (br s, 5H), 2.95-2.84 (m, 1H), 2.62-2.51 (m, 6H), 2.39-2.32 (m, 1H), 2.21 (br d, J=6.4 Hz, 2H), 1.99-1.92 (m, 1H), 1.90-1.84 (m, 1H), 1.79 (br d, J=10.3 Hz, 3H), 1.65 (br s, 2H), 1.59-1.48 (m, 2H), 1.19 (br d, J=10.0 Hz, 2H), 1.04 (s, 5H), 0.91 (br d, J=10.6 Hz, 3H), 0.73-0.61 (m, 1H).

### Example 70. Preparation of (I-257) (S)-3-(5-(4-((1-(4-((3R,4S)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione

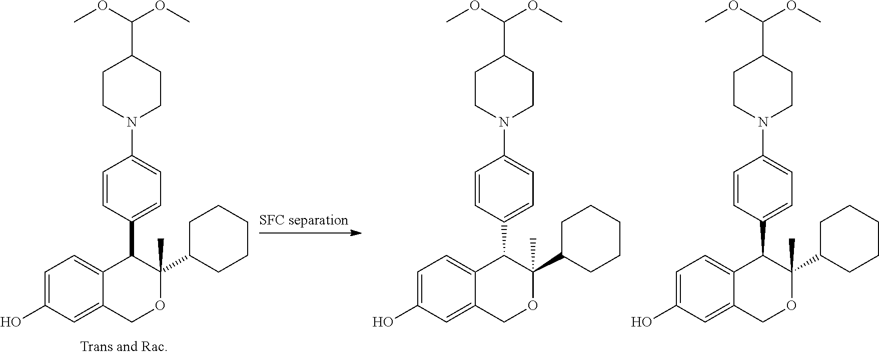 [SMILES: COC(OC)C1CCN(c2ccc([C@@H]3c4ccc(O)cc4CO[C@@]3(C)C3CCCCC3)cc2)CC1 ; COC(OC)C1CCN(c2ccc([C@H]3c4ccc(O)cc4CO[C@]3(C)C3CCCCC3)cc2)CC1 ; COC(OC)C1CCN(c2ccc([C@@H]3c4ccc(O)cc4CO[C@@]3(C)C3CCCCC3)cc2)CC1]

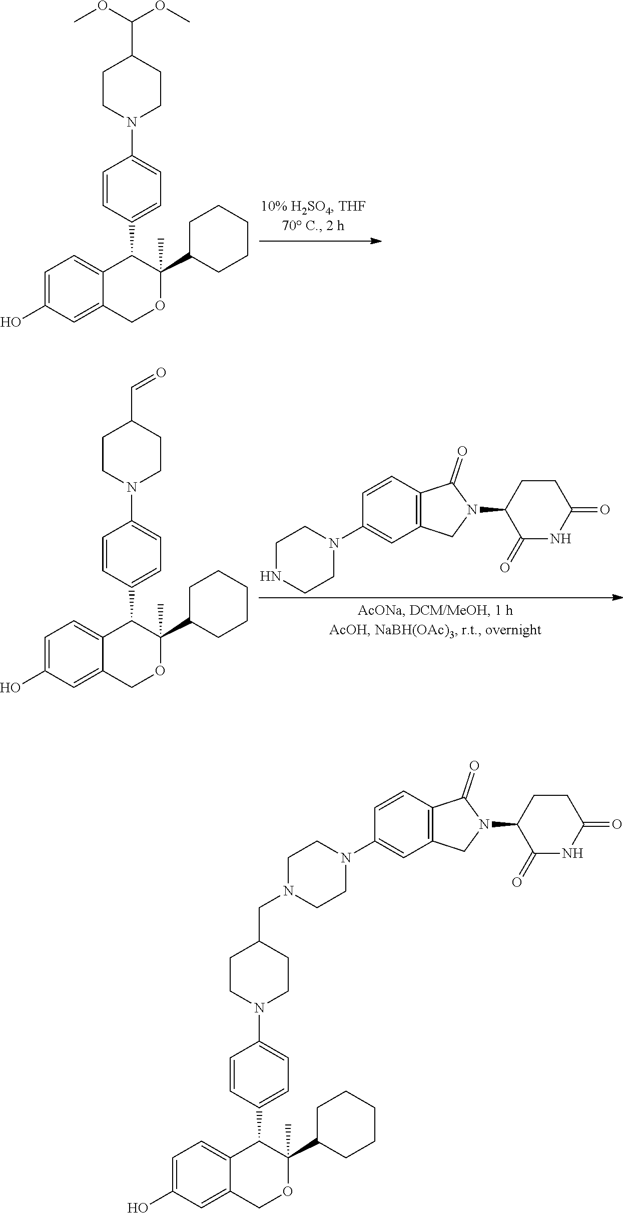

Step 1: The white solid was purified by SFC. Condition: CO₂-EtOH (0.1% NH₃H₂O); Column: DAICEL CHIRALPAK AD (250 mm*30 mm, 10 um), Begin B: 50%, End B: 50%, FlowRate (ml/min): δ 0. The pure fractions were collected and the solvent was evaporated under vacuum. The aqueous layer was lyophilized to dryness to give (3R,4S)-3-cyclohexyl-4-(4-(4-(dimethoxymethyl)piperidin-1-yl)phenyl)-3-methylisochroman-7-ol (15 mg, 30 μmol, 14%, 96.62% purity) as a colorless oil and ((3S,4R)-3-cyclohexyl-4-(4-(4-(dimethoxymethyl)piperidin-1-yl)phenyl)-3-methylisochroman-7-ol (11 mg, 23 μmol, 11%, 100% purity) as a colorless oil.

LC-MS (ESI+) m/z: 480.2 (M+H)⁺.

Step 2: To a solution of (3R,4S)-3-cyclohexyl-4-(4-(4-(dimethoxymethyl)piperidin-1-yl)phenyl)-3-methylisochroman-7-ol (15 mg, 1 Eq, 30 μmol) in THF (2 mL) was added sulfuric acid (30 mg, 2 mL, 10% Wt, 1 Eq, 30 μmol). The mixture was stirred at 70° C. for 1 hour. The mixture was adjusted to pH 8-9 by progressively adding NaHCO₃. The reaction mixture was quenched by addition 100 mL EtOAc at 20° C., and then diluted with 50 m H₂OL and extracted with EtOAc (25 mL*2). The combined organic layers were washed with 10 mL, dried over Na₂SO₄. The reaction was filtered and concentrated under reduced pressure to give 1-(4-((3R,4S)-3-cyclohexyl-7-hydroxy-3-methylisochroman-4-yl)phenyl)piperidine-4-carbaldehyde (25 mg, 56 μmol, 180%, 96.5% purity) as a colorless oil. LC-MS (ESI+) m/z: 452.2 (M+18)⁺.

Step 3: A mixture of 1-(4-((3R,4S)-3-cyclohexyl-7-hydroxy-3-methylisochroman-4-yl)phenyl)piperidine-4-carbaldehyde (25 mg, 96.5% Wt, 1 Eq, 56 μmol), (S)-3-(1-oxo-5-(piperazin-1-yl)isoindolin-2-yl)piperidine-2,6-dione PhSO₃H salt (18 mg, 1 Eq, 56 μmol) and sodium acetate (23 mg, 5 Eq, 0.28 mmol) in DCM (2 mL) and MeOH (2 mL) at 20° C. for 0.5 hour, pH=8, then acetic acid (10 mg, 9.5 μL, 3 Eq, 0.17 mmol) at 20° C. for 1 hour, pH=6, then added Sodium triacetoxyborohydride (24 mg, 2 Eq, 0.11 mmol) the mixture, and then the mixture was stirred at 25° C. for 16 hour. The reaction was quenched with water (50 mL) and extracted with ethyl acetate (50 mL*2). The reaction was concentrated to give (S)-3-(5-(4-((1-(4-((3R,4S)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione as a colorless oil. The colorless oil was purified by preparative high-performance liquid chromatography. Condition: water (FA)-CAN. Column: Boston Green ODS 150*30 mm*5 um. Begin B (20%), End: B (50%). Gradient Time (min) 6; 100% B hold Time (min) 1, Flow Rate (ml/min) 30. The pure fractions were collected and the solvent was evaporated under vacuum. The aqueous layer was lyophilized to dryness to give (S)-3-(5-(4-((1-(4-((3R,4S)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione (15.9 mg, 21.1 μmol, 38%, 98.89% Purity) as a white solid.

LCMS: calc. for C₄₄H₅₅N₅O₅: 745.42, found: [M+H]⁺ 746.5.

HPLC: 98.89% purity at 220 nm.

¹H NMR (400 MHZ, DMSO-d₆) δ=10.95 (s, 1H), 9.25-9.12 (m, 1H), 8.19-8.10 (m, 0.343H), 7.55-7.47 (m, 1H), 7.07 (s, 2H), 6.91 (br d, J=7.7 Hz, 2H), 6.76 (br d, J=8.8 Hz, 2H), 6.69 (d, J=8.5 Hz, 1H), 6.51-6.43 (m, 2H), 5.09-5.01 (m, 1H), 4.77-4.70 (m, 1H), 4.64-4.57 (m, 1H), 4.36-4.29 (m, 1H), 4.24-4.16 (m, 1H), 3.81 (s, 1H), 3.59 (br d, J=11.0 Hz, 2H), 3.28 (br 8, 5H), 2.96-2.84 (m, 1H), 2.68-2.53 (m, 6H), 2.40-2.32 (m, 1H), 2.21 (br d, J=7.0 Hz, 2H), 1.99-1.92 (m, 1H), 1.83-1.76 (m, 3H), 1.76-1.64 (m, 4H), 1.62-1.55 (m, 1H), 1.53-1.43 (m, 1H), 1.25-1.17 (m, 2H), 1.16-1.06 (m, 3H), 1.02 (br s, 2H), 0.68 (s, 3H).

### Example 71. Preparation of (I-258) (S)-3-(5-(4-((1-(4-((3S,4R)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione

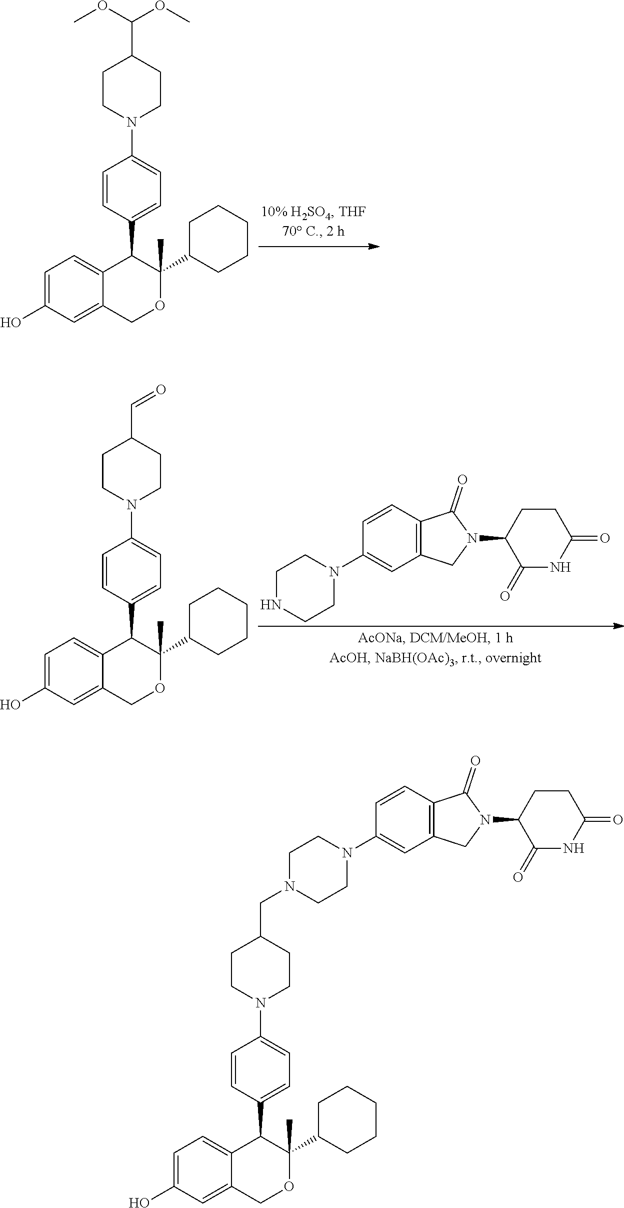

Step 1: To a solution of (3S,4R)-3-cyclohexyl-4-(4-(4-(dimethoxymethyl)piperidin-1-yl)phenyl)-3-methylisochroman-7-ol (11 mg, 1 Eq, 23 μmol) in THF (2 mL) was added sulfuric acid (22 mg, 2 mL, 10% Wt, 1 Eq, 23 μmol). The mixture was stirred at 70° C. for 1 hour. The mixture was adjusted to pH 8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL, dried over Na₂SO₄. The reaction was filtered and concentrated under reduced pressure to give 1-(4-((3S,4R)-3-cyclohexyl-7-hydroxy-3-methylisochroman-4-yl)phenyl)piperidine-4-carbaldehyde (15 mg, 33 μmol, 140%, 94.9% purity) as a colorless oil. LC-MS (ESI+) m/z: 452.2 (M+18)⁺.

Step 2: A mixture of 1-(4-((3S,4R)-3-cyclohexyl-7-hydroxy-3-methylisochroman-4-yl)phenyl)piperidine-4-carbaldehyde (15 mg, 94.9% Wt, 1 Eq, 33 μmol), (S)-3-(1-oxo-5-(piperazin-1-yl)isoindolin-2-yl)piperidine-2,6-dione PhSO₃H salt (11 mg, 1 Eq, 33 μmol) and sodium acetate (13 mg, 5 Eq. 0.16 mmol) in DCM (2 mL) and MeOH (2 mL) at 20° C. for 0.5 hour, pH=8, then acetic acid (5.9 mg, 5.6 μL, 3 Eq, 98 μmol) at 20° C. for 1 hour, pH=6, then added Sodium triacetoxyborohydride (14 mg, 2 Eq, 66 μmol) the mixture, and then the mixture was stirred at 25° C. for 16 hour. The reaction was quenched with water (50 mL) and extracted with ethyl acetate (50 mL*2). The reaction was concentrated to give (S)-3-(5-(4-((1-(4-((3S,4R)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione as a colorless oil.

The colorless oil was purified by preparative high-performance liquid chromatography. Condition: water (FA)-CAN. Column: Boston Green ODS 150*30 mm*5 um. Begin B (25%), End: B (55%), Gradient Time (min) 6; 100% B hold Time (min) 1, Flow Rate (ml/min) 30. The pure fractions were collected, and the solvent was evaporated under vacuum. The aqueous layer was lyophilized to dryness to give (S)-3-(5-(4-((1-(4-((3S,4R)-3-cyclohexyl-7-hydroxy-3-methylisochroman-4-yl)phenyl)piperidin-4-yl)methyl)piperazin-1-yl)-1-oxoisoindolin-2-yl)piperidine-2,6-dione (6.4 mg, 8.5 μmol, 26%, 99.03% purity) as a white solid.

LCMS: calc. for C₄₄H₅₅N₅O₅: 745.42, found: [M+H]⁺ 746.4.

HPLC: 99.03% purity at 220 nm.

¹H NMR (400 MHZ, DMSO-d₆) δ=10.98-10.93 (m, 1H), 9.22-9.16 (m, 1H), 8.20-8.17 (m, 0.413H), 7.54-7.49 (m, 1H), 7.06 (s, 2H), 6.94-6.86 (m, 2H), 6.79-6.74 (m, 2H), 6.71-6.66 (m, 1H), 6.51-6.43 (m, 2H), 5.08-5.01 (m, 1H), 4.78-4.69 (m, 1H), 4.64-4.56 (m, 1H), 4.36-4.29 (m, 1H), 4.24-4.16 (m, 1H), 3.83-3.78 (m, 1H), 3.62-3.56 (m, 2H), 3.30-3.25 (m, 6H), 2.93-2.84 (m, 1H), 2.69-2.55 (m, 6H), 2.36-2.32 (m, 1H), 2.24-2.18 (m, 2H), 1.99-1.91 (m, 1H), 1.83-1.76 (m, 3H), 1.74-1.67 (m, 3H), 1.61-1.55 (m, 1H), 1.53-1.44 (m, 1H), 1.24-1.17 (m, 2H), 1.15-1.06 (m, 3H), 1.05-0.96 (m, 2H), 0.68 (s, 3H).

### Example 72. Preparation of (I-267) (S)-3-(5-(4-((1-(4-((1R,4′S)-7′-hydroxy-2,3-dihydrospiro[indene-1,3′-isochroman]-4′-yl)phenyl)piperidin-4-yl)methyl)piperazin-1-yl)-1-oxoisoindolin-2-yl)piperidine-2,6-dione (18.5 mg, 24.6 μmol, 27% yield)

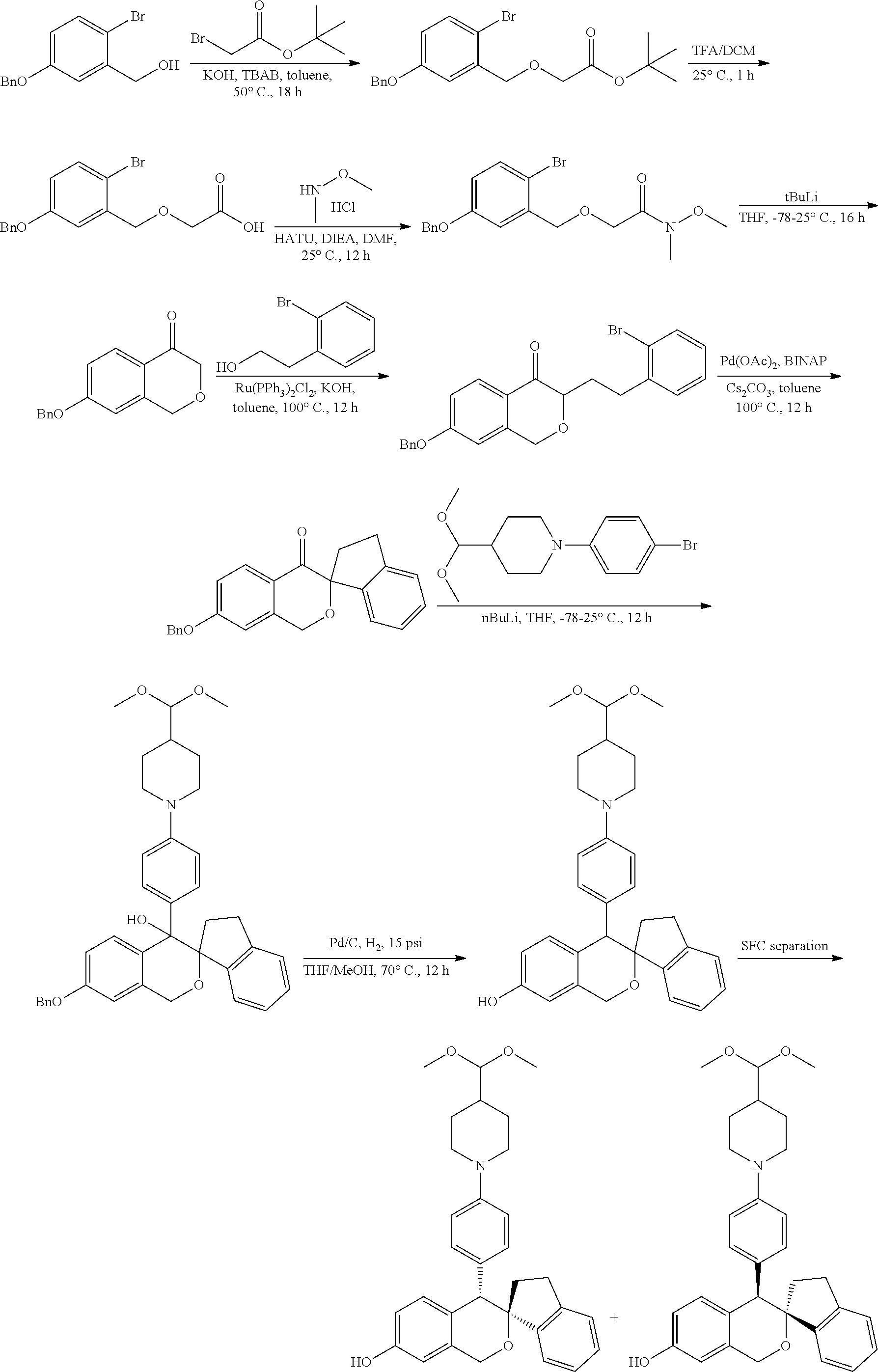

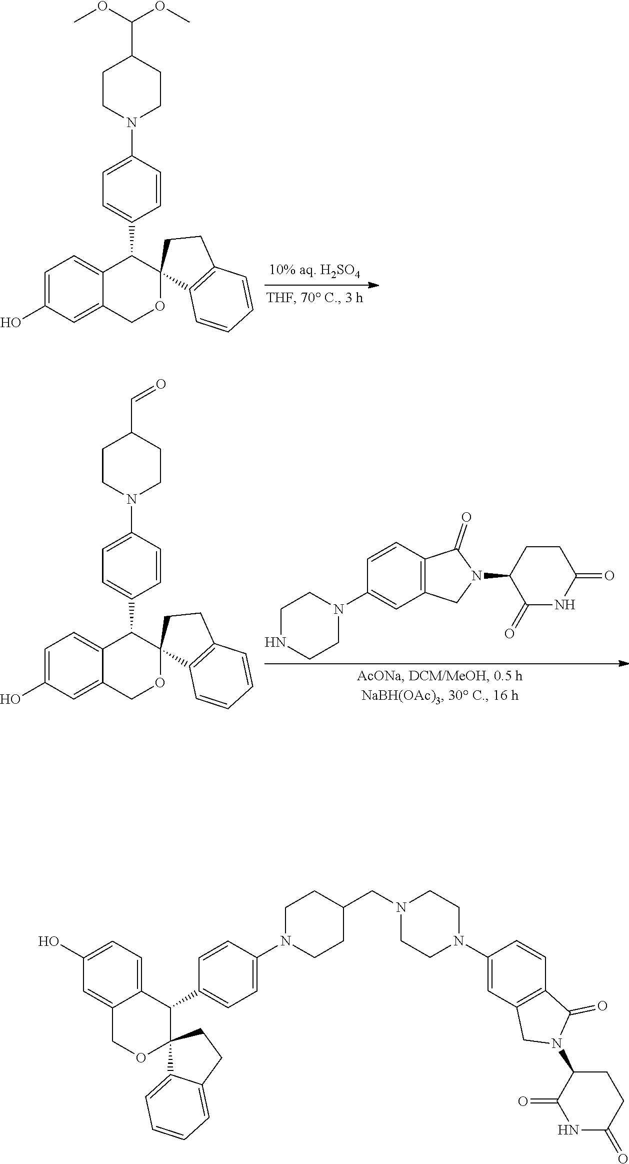

Step 1: A mixture of (5-(benzyloxy)-2-bromophenyl) methanol (30 g, 102 mmol, 1 eq), tert-butyl 2-bromoacetate (24 g, 123 mmol, 18 mL, 1.2 eq), KOH (14 g, 102 mmol, 240 mL, 40% purity, 1 eq), TBAB (3 g, 9.3 mmol, 0.09 eq) was added in toluene (200 mL), the mixture was stirred at 50° C. for 16 h. TLC (petroleum ether:ethyl acetate=10:1, R_{f}=0.7 UV) showed one main new spot was observed. The resulting solution was diluted with H₂O (200 mL) and was extracted with Ethyl acetate (300 mL*2), the organic layers were dried with anhydrous Na₂SO₄, filtered, and concentrated to give the crude product. The residue was purified by column chromatography (SiO₂, Petroleum ether/Ethyl acetate=10/0 to 9/1). Compound tert-butyl 2-((5-(benzyloxy)-2-bromobenzyl)oxy)acetate (34 g, 83 mmol, 82% yield) was obtained as a yellow oil.

Step 2: A mixture of tert-butyl 2-((5-(benzyloxy)-2-bromobenzyl)oxy)acetate (34 g, 83 mmol, 1 eq) was added in TFA (150 mL) and DCM (150 mL) the mixture was stirred at 25° C. for 1 hr. TLC (petroleum ether:ethyl acetate=10:1, R_{f}=0.2 UV) showed one main new spot was observed. The resulting solution was quenched with saturated aqueous NaHCO₃ and adjusted pH to 8 at 0° C., was treated with 200 mL H₂O extracted with ethyl acetate (500 mL*2), The organic layers were dried with anhydrous Na₂SO₄, filtered, and concentrated to give the crude product. The crude product 2-((5-(benzyloxy)-2-bromobenzyl)oxy) acetic acid (1.6 g, 4.6 mmol, 96% yield) was used into the next step without further purification. Compound 2-((5-(benzyloxy)-2-bromobenzyl)oxy) acetic acid (24 g, 68.3 mmol, 82% yield) was obtained as a red oil.

Step 3: A mixture of 2-((5-(benzyloxy)-2-bromobenzyl)oxy) acetic acid (24 g, 68.3 mmol, 1 eq), N,O-dimethylhydroxylamine hydrogen chloride (6.7 g, 68 mmol, 1 eq), HATU (39 g, 102.5 mmol, 1.5 eq), DIEA (26.5 g, 205 mmol, 35.7 mL, 3 eq) in DMF (200 mL) and was stirred at 25° C. for 12 h. TLC (petroleum ether:ethyl acetate=10:1, R_{f}=0.6 UV) showed one main new spot was observed. The resulting solution was diluted with 300 mL H₂O and was extracted with Ethyl acetate (600 mL*2), the organic layers were dried with anhydrous Na₂SO₄, filtered, and concentrated to give the crude product. The residue was purified by column chromatography (SiO₂, Petroleum ether/Ethyl acetate=10/0 to 9/1). Compound 2-((5-(benzyloxy)-2-bromobenzyl)oxy)-N-methoxy-N-methylacetamide (20 g, 50.7 mmol, 74% yield) was obtained as a white solid.

Step 4: A solution of 2-((5-(benzyloxy)-2-bromobenzyl)oxy)-N-methoxy-N-methylacetamide (4 g, 1 Eq, 10.1 mmol) in THF (100 mL) was stirred at −78° C. for 10 min under N₂ atmosphere, then added tert-butyllithium (1.5 g, 18 mL, 1.3 molar, 2.3 Eq, 23 mmol) at −78° C. The reaction was stirred at −78° C. to 25° C. for 16 hour under N₂ atmosphere. TLC (petroleum ether:ethyl acetate=5:1, UV) showed one main new spot was observed. The reaction was quenched with aq. NH₄Cl (100 mL) and extracted with ethyl acetate (100 mL*3). The organic layer was washed with brine (100 mL) and dried over Na₂SO₄ and concentrated in vacuum to give crude product. The black oil was subjected to column chromatography over silica gel (gradient elution: 0-20% ethyl acetate). The desired fractions were collected, and concentrated to dryness in vacuum to give 7-(benzyloxy)isochroman-4-one (2.3 g, 9 mmol, 89% yield) as a yellow solid.

Step 5: A mixture of 7-(benzyloxy)isochroman-4-one (1.6 g, 6.3 mmol, 1 eq), 2-(2-bromophenyl) ethanol (1.5 g, 7.6 mmol, 1 mL, 1.2 eq), KOH (353 mg, 6.3 mmol, 1 eq), Ru(PPh₃)₂Cl₂ (575 mg, 629 μmol, 0.1 eq) was added in toluene (10 mL), the mixture was stirred at 100° C. for 12 h. TLC (petroleum ether:ethyl acetate=10:1, R_{f}=0.7 UV) showed one main new spot was observed. The resulting solution was diluted with H₂O (50 mL) and was extracted with ethyl acetate (100 mL*2), the organic layers were dried with anhydrous Na₂SO₄, filtered, and concentrated to give the crude product. The residue was purified by column chromatography (SiO₂, Petroleum ether/Ethyl acetate=10/0 to 9/1). Compound 7-(benzyloxy)-3-(2-bromophenethyl)isochroman-4-one (1.2 g, 2.7 mmol, 43% yield) was obtained as a yellow oil.

Step 6: A mixture of 7-(benzyloxy)-3-(2-bromophenethyl)isochroman-4-one (1.2 g, 2.7 mmol, 1 eq), dicesium carbonate (1.8 g, 5.4 mmol, 2 eq), Pd(OAc)₂ (61 mg, 272 μmol, 0.1 eq), [1-(2-diphenylphosphanyl-1-naphthyl)-2-naphthyl]-diphenyl-phosphane (254 mg, 408 μmol, 0.15 eq) was added in toluene (10 mL) and was purged N₂ 3 times, the mixture was stirred at 100° C. for 12 h. TLC (petroleum ether:ethyl acetate=10:1, R_{f}=0.7 UV) showed one main new spot was observed. The resulting solution was quenched with 50 mL H₂O and was extracted with ethyl acetate (100 mL*2), the organic layers were dried with anhydrous Na₂SO₄, filtered, and concentrated to give the crude product. The residue was purified by column chromatography (SiO₂, Petroleum ether/Ethyl acetate=10/0 to 9/1). Compound 7′-(benzyloxy)-2,3-dihydrospiro[indene-1,3′-isochroman]-4′-one (700 mg, 2 mmol, 72% yield) was obtained as a yellow oil.

Step 7: n-Butyllithium (2.5 M, 2.4 mL, 3 eq) was added into mixture of 1-(4-bromophenyl)-4-(dimethoxymethyl)piperidine (2 g, 6.4 mmol, 3.2 eq) in THF (10 mL) under N₂ at −78° C., and the mixture was stirred at −78° C. for 0.5 h under N₂ atmosphere, and 7′-(benzyloxy)-2,3-dihydrospiro[indene-1,3′-isochroman]-4′-one (700 mg, 1.9 mmol, 1 eq) was added in the mixture at −78° C. and was stirred at 25° C. for 12 h. TLC (petroleum ether:ethyl acetate=10:1, R_{f}=0.7 UV) showed one main new spot was observed. The resulting solution was quenched with saturated aqueous NH₄Cl (20 mL) at 0° C. and diluted with H₂O (50 mL) and was extracted with ethyl acetate (100 mL), The organic layers were washed with brine (50 mL), dried with anhydrous Na₂SO₄, filtered, and concentrated to give the crude product. The residue was purified by column chromatography (SiO₂, Petroleum ether/Ethyl acetate=10/0 to 9/1). Compound 7′-(benzyloxy)-4′-(4-(4-(dimethoxymethyl)piperidin-1-yl)phenyl)-2,3-dihydrospiro[indene-1,3′-isochroman]-4′-ol (1.1 g, 1.9 mmol, 95% yield) was obtained as a yellow oil.

Step 8: A mixture of 7′-(benzyloxy)-4′-(4-(4-(dimethoxymethyl)piperidin-1-yl)phenyl)-2,3-dihydrospiro[indene-1,3′-isochroman]-4′-ol (500 mg, 845 μmol, 1 eq), Pd/C (500 mg, 470 μmol, 10% purity,), Pd(OH) 2/C (500 mg, 3.6 mmol) was added in THF (10 mL) and MeOH (10 mL), the mixture was stirred at 70° C. for 12 h. TLC (petroleum ether:ethyl acetate=10:1, R_{f}=0.7, UV) showed one main new spot was observed. The reaction mixture was filtered and concentrated under reduced pressure to give a residue. The crude product was purified by reversed-phase SFC (column: DAICEL CHIRALCEL OJ (250 mm*30 mm, 10 um); mobile phase: [CO₂-MeOH (0.1% NH₃H₂O)]; B %: 35% %, isocratic elution mode retention time 3.213,3.534) and by HPLC (column: Xtimate C18 150*40 mm*5 um; mobile phase: [water (FA)-ACN]; gradient: 25%-65% B over 9 min). Compound (1R,4′S)-4′-(4-(4-(dimethoxymethyl)piperidin-1-yl)phenyl)-2,3-dihydrospiro[indene-1,3′-isochroman]-7′-ol (100 mg, 205.9 μmol, 24% yield) and (1S,4′R)-4′-(4-(4-(dimethoxymethyl)piperidin-1-yl)phenyl)-2,3-dihydrospiro[indene-1,3′-isochroman]-7′-ol (120 mg, 247.1 μmol, 29% yield) was obtained as a white solid. LC-MS (ESI+) m/z: 486.2 (M+H)⁺.

Step 9: To a solution of (1R,4′S)-4′-(4-(4-(dimethoxymethyl)piperidin-1-yl)phenyl)-2,3-dihydrospiro[indene-1,3′-isochroman]-7′-ol (50 mg, 103 μmol, 1 eq) in 10% H₂SO₄ (4 mL) and THF (4 mL). The mixture was stirred at 70° C. for 1 h. TLC (petroleum ether:ethyl acetate=2:1, R_{f}=0.3 UV) showed one main new spot was observed. The reaction mixture was quenched by saturated aqueous NaHCO₃ (20 mL) at 25° C., and then diluted with H₂O (20 mL) and extracted with ethyl acetate (40 mL). The combined organic layers were washed with brine (20 mL), dried over anhydrous Na₂SO₄, filtered and concentrated under reduced pressure to give a residue. The crude product 1-(4-((1R,4′S)-7′-hydroxy-2,3-dihydrospiro[indene-1,3′-isochroman]-4′-yl)phenyl)piperidine-4-carbaldehyde (40 mg, 91 μmol, 88% yield) was used into the next step without further purification. Compound 1-(4-((1R,4′S)-7′-hydroxy-2,3-dihydrospiro[indene-1,3′-isochroman]-4′-yl)phenyl)piperidine-4-carbaldehyde (40 mg, 91 μmol, 88% yield) was obtained as a yellow oil.

LC-MS (ESI+) m/z: 440.1 (M+H)⁺

Step 10: To a solution of 1-(4-((1R,4′S)-7-hydroxy-2,3-dihydrospiro[indene-1,3′-isochroman]-4′-yl)phenyl)piperidine-4-carbaldehyde (40 mg, 91 μmol, 1 eq) and (3S)-3-(1-oxo-5-piperazin-1-yl-isoindolin-2-yl)piperidine-2,6-dione (46 mg, 91 μmol, 1 eq, PhSO₃H salt) in DCM (4 mL) and MeOH (4 mL) was added sodium acetate (22 mg, 273 μmol, 3 eq). The mixture was stirred at 30° C. for 0.5 h. Then the mixture was added NaBH(OAc)₃ (39 mg, 182 μmol, 2 eq) and acetic acid (16 mg, 273 μmol, 16 μL, 3 eq). The mixture was stirred at 30° C. for 16 h finally. TLC (Dichloromethane:Methanol=10:1, R_{f}=0.3 UV) showed one main new spot was observed. The reaction mixture was diluted with H₂O (20 mL) and extracted with DCM (20 mL). The combined organic layers were washed with brine 20 mL, dried over Na₂SO₄, filtered and concentrated under reduced pressure to give a residue. The residue was purified by prep-HPLC (column: Welch Xtimate C18 150*30 mm*5 um; mobile phase: [water (FA)-ACN]: gradient: 15%-45% B over 7 min). (S)-3-(5-(4-((1-(4-((1R,4′S)-7′-hydroxy-2,3-dihydrospiro[indene-1,3′-isochroman]-4′-yl)phenyl)piperidin-4-yl)methyl)piperazin-1-yl)-1-oxoisoindolin-2-yl)piperidine-2,6-dione (18.5 mg, 24.6 μmol, 27% yield) was obtained as a white solid.

LC-MS (ESI⁺) m/z: 752.5 (M+H)⁺.

HPLC: 97%, purity at 220 nm.

¹H NMR (400 MHZ, DMSO-d₆) δ ppm 10.96 (s, 1H) 9.25-9.32 (m, 1H) 8.14 (s, 0.413H) 7.53 (d, J=8.34 Hz, 1H) 7.16-7.20 (m, 1H) 7.05-7.13 (m, 3H) 6.80 (t, J=7.27 Hz, 1H) 6.72 (br d, J=8.82 Hz, 1H) 6.65 (br s, 4H) 6.53-6.58 (m, 2H) 6.18 (d, J=7.75 Hz, 1H) 5.05 (dd, J=13.11, 5.01 Hz, 1H) 4.84-4.98 (m, 2H) 4.28-4.38 (m, 1H) 4.16-4.25 (m, 1H) 3.80 (s, 1H) 3.58 (br d, J=9.78 Hz, 2H) 3.36-3.43 (m, 8H) 2.84-2.93 (m, 3H) 2.55-2.60 (m, 4H) 2.31-2.43 (m, 2H) 2.19-2.24 (m, 1H) 2.07-2.14 (m, 1H) 1.92-2.00 (m, 1H) 1.66-1.81 (m, 3H) 1.16-1.27 (m, 2H).

### Example 73. Preparation of (I-77) (S)-3-(5-(4-((1-(4-((1S,4′R)-7′-hydroxy-2,3-dihydrospiro[indene-1,3′-isochroman]-4′-yl)phenyl)piperidin-4-yl)methyl)piperazin-1-yl)-1-oxoisoindolin-2-yl)piperidine-2,6-dione

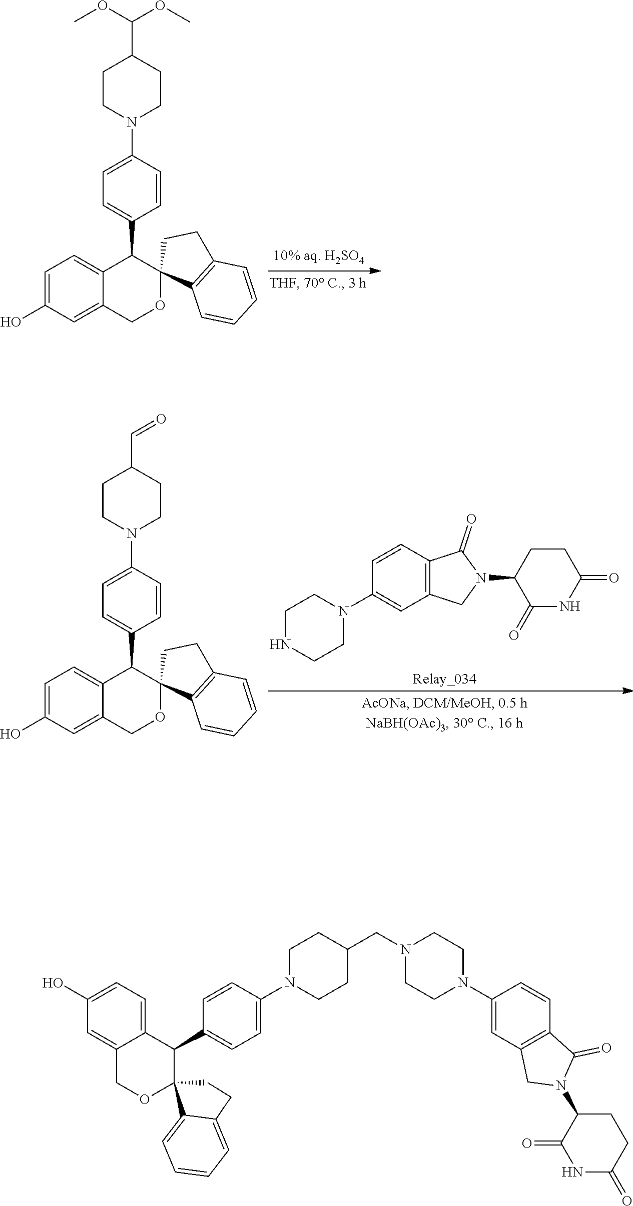 [SMILES: COC(OC)C1CCN(c2ccc([C@@H]3c4ccc(O)cc4CO[C@@]34CCc3ccccc34)cc2)CC1 ; O=CC1CCN(c2ccc([C@@H]3c4ccc(O)cc4CO[C@@]34CCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@]78CCc7ccccc78)cc6)CC5)CC4)ccc3C2=O)C(=O)N1]

Step 1: To a solution of (1S,4′R)-4′-(4-(4-(dimethoxymethyl)piperidin-1-yl)phenyl)-2,3-dihydrospiro[indene-1,3′-isochroman]-7′-ol (60 mg, 123 μmol, 1 eq) in 10% H₂SO₄ (4 mL) and THF (4 mL). The mixture was stirred at 70° C. for 1 h. TLC (petroleum ether:ethyl acetate=2:1, R_{f}=0.3 UV) showed one main new spot was observed. The reaction mixture was quenched by saturated aqueous NaHCO₃ (20 mL) at 25° C., and then diluted with 20 mL H₂O and extracted with 40 mL ethyl acetate. The combined organic layers were washed with 20 mL brine, dried over anhydrous Na₂SO₄, filtered and concentrated under reduced pressure to give a residue. The crude product 1-(4-((1S,4′R)-7′-hydroxy-2,3-dihydrospiro[indene-1,3′-isochroman]-4′-yl)phenyl)piperidine-4-carbaldehyde (40 mg, 91 μmol, 74% yield) was used into the next step without further purification. Compound 1-(4-((1S,4′R)-7′-hydroxy-2,3-dihydrospiro[indene-1,3′-isochroman]-4′-yl)phenyl)piperidine-4-carbaldehyde (40 mg, 91 μmol, 74% yield) was obtained as a yellow oil.

LC-MS (ESI+) m/z: 458.2 (M+H₂O)⁺.

Step 2: To a solution of 1-(4-((1S,4′R)-7-hydroxy-2,3-dihydrospiro[indene-1,3′-isochroman]-4′-yl)phenyl)piperidine-4-carbaldehyde (40 mg, 91 μmol, 1 eq) and (3S)-3-(1-oxo-5-piperazin-1-yl-isoindolin-2-yl)piperidine-2,6-dione (45.6 mg, 91 μmol, 1 eq, PhSO₃H salt) in DCM (4 mL) and MeOH (4 mL) was added sodium acetate (22 mg, 273 μmol, 3 eq). The mixture was stirred at 30° C. for 0.5 h. Then the mixture was added NaBH(OAc)₃ (39 mg, 182 μmol, 2 eq) and acetic acid (16 mg, 273 μmol, 16 μL, 3 eq). The mixture was stirred at 30° C. for 16 h finally. TLC (DCM:Methanol=10:1, R_{f}=0.3, UV) showed one main new spot was observed. The reaction mixture was diluted with H₂O (10 mL) and extracted with DCM (20 mL*2). The combined organic layers were washed with brine (10 mL*2), dried over Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 15%-45% B over 7 min). (S)-3-(5-(4-((1-(4-((1S,4′R)-7′-hydroxy-2,3-dihydrospiro[indene-1,3′-isochroman]-4′-yl)phenyl)piperidin-4-yl)methyl)piperazin-1-yl)-1-oxoisoindolin-2-yl)piperidine-2,6-dione (21.4 mg, 28 μmol, 31% yield) was obtained as a white solid.

LC-MS (ESI+) m/z: 752.4 (M+H)⁺.

HPLC: 95%, purity at 220 nm.

¹H NMR (400 MHZ, DMSO-d₆) δ ppm 10.96 (s, 1H) 9.28 (s, 1H) 8.12-8.19 (m, 0.379H) 7.54 (d, J=8.34 Hz, 1H) 7.15-7.24 (m, 1H) 7.03-7.13 (m, 3H) 6.77-6.87 (m, 1H) 6.61-6.75 (m, 5H) 6.50-6.57 (m, 2H) 6.13-6.23 (m, 1H) 5.02-5.13 (m, 1H) 4.84-4.99 (m, 2H) 4.16-4.39 (m, 2H) 3.76-3.84 (m, 1H) 3.55-3.63 (m, 2H) 3.35-3.40 (m, 8H) 2.84-2.94 (m, 3H) 2.55-2.63 (m, 4H) 2.30-2.44 (m, 2H) 2.18-2.25 (m, 1H) 2.06-2.14 (m, 1H) 1.92-2.01 (m, 1H) 1.78 (br d, J=11.80 Hz, 3H) 1.17-1.27 (m, 2H).

### Example 74. Preparation of (I-346) (S)-3-(5-(4-((1-(4-((3R,4S)-3-(4-fluorophenyl)-7-hydroxyisochroman-4-yl)phenyl)piperidin-4-yl)methyl)piperazin-1-yl)-1-oxoisoindolin-2-yl)piperidine-2,6-dione

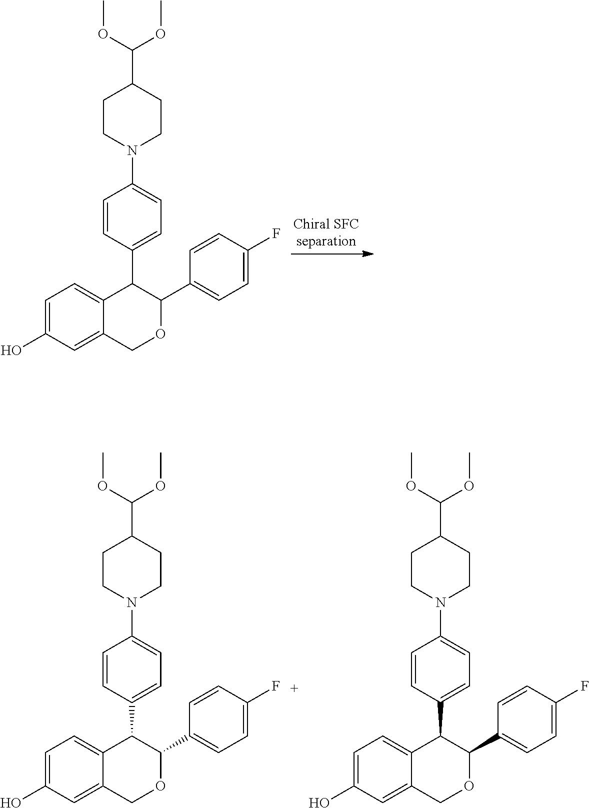 [SMILES: COC(OC)C1CCN(c2ccc(C3c4ccc(O)cc4COC3c3ccc(F)cc3)cc2)CC1 ; COC(OC)C1CCN(c2ccc([C@H]3c4ccc(O)cc4CO[C@H]3c3ccc(F)cc3)cc2)CC1 ; COC(OC)C1CCN(c2ccc([C@@H]3c4ccc(O)cc4CO[C@@H]3c3ccc(F)cc3)cc2)CC1]

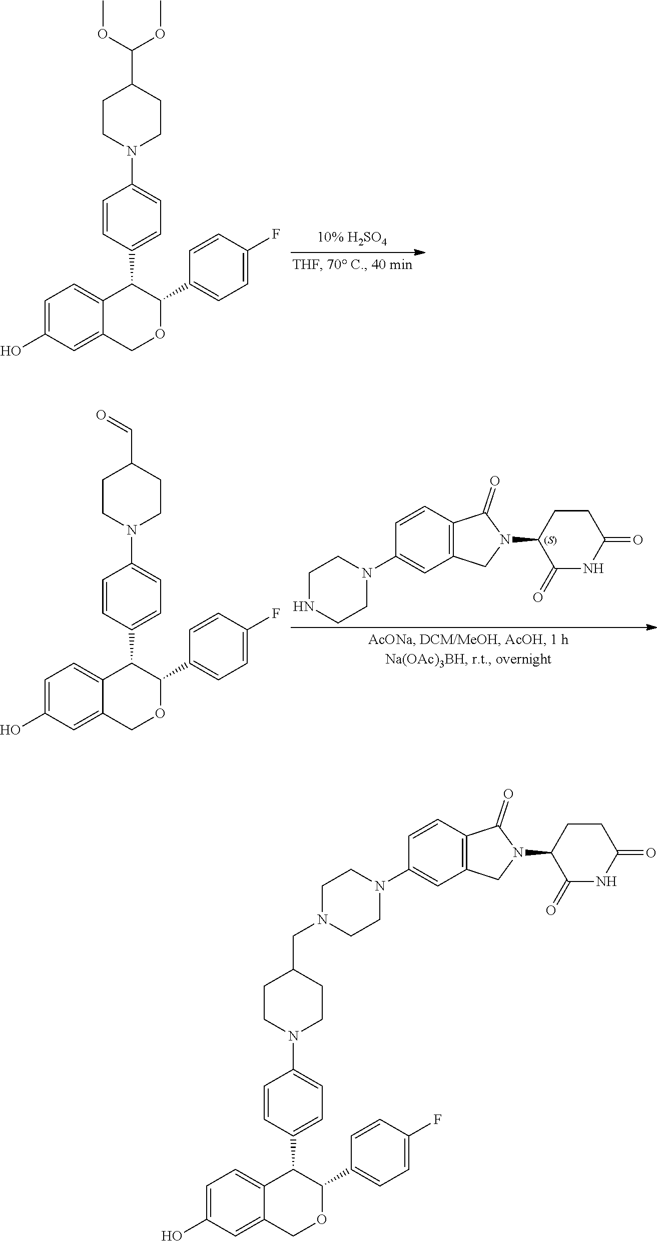

Step 1: The white solid was purified by SFC. Column: DAICEL CHIRALCEL OJ (250 mm*30 mm, 10 um); Condition: CO₂-EtOH (0.1% NH₃H₂O); At the beginning: B (30%); At the end: B (30%); Flow Rate (ml/min) 80. The aqueous phase was lyophilized to dryness to give (3R,4S)-4-(4-(4-(dimethoxymethyl)piperidin-1-yl)phenyl)-3-(4-fluorophenyl)isochroman-7-ol (52 mg, 0.10 mmol, 38%, 95.5% purity) as a colorless oil and (3S,4R)-4-(4-(4-(dimethoxymethyl)piperidin-1-yl)phenyl)-3-(4-fluorophenyl)isochroman-7-ol (63 mg, 0.13 mmol, 46%, 95.1% purity) as a colorless oil.

LC-MS (ESI+) m/z: 478.1 (M+H)⁺.

Step 2: To a solution of (3R,4S)-4-(4-(4-(dimethoxymethyl)piperidin-1-yl)phenyl)-3-(4-fluorophenyl)isochroman-7-ol (52 mg, 1 Eq, 0.10 mmol) in THF (5 mL) was added 10% sulfuric acid (0.10 g, 5 mL). The mixture was stirred at 70° C. for 30 min. The mixture was adjusted to pH 8-9 by progressively adding NaHCO₃. The reaction mixture was quenched by addition 100 mL ethyl acetate at 20° C., and then diluted with 50 mL H₂O and extracted with ethyl acetate (25 mL*2). The combined organic layers were washed with 10 mL brine, dried over Na₂SO₄. The reaction was filtered and concentrated under reduced pressure to give 1-(4-((3R,4S)-3-(4-fluorophenyl)-7-hydroxyisochroman-4-yl)phenyl)piperidine-4-carbaldehyde (80 mg, 0.17 mmol, 90.77% purity) as a colorless oil. LC-MS (ESI+) m/z: 450.2 (M+18)⁺.

Step 3: A mixture of 1-(4-((3R,4S)-3-(4-fluorophenyl)-7-hydroxyisochroman-4-yl)phenyl)piperidine-4-carbaldehyde (80 mg, 1 Eq, 0.17 mmol), (S)-3-(1-oxo-5-(piperazin-1-yl)isoindolin-2-yl)piperidine-2,6-dione benzenesulfonate (82 mg, 1 Eq, 0.17 mmol) and sodium acetate (69 mg, 5 Eq, 0.84 mmol) in DCM (2.5 mL) and MeOH (2.5 mL) at 20° C. for 0.5 hour, pH=8, then acetic acid (30 mg, 29 μL, 3 Eq, 0.50 mmol) at 20° C. for 1 hour, pH=6, then added sodium triacetoxyborohydride (71 mg, 2 Eq, 0.34 mmol) the mixture, and then the mixture was stirred at 25° C. for 16 hour. The reaction was quenched with water (50 mL) and extracted with ethyl acetate (50 mL*2). The reaction was concentrated to give (S)-3-(5-(4-((1-(4-((3*S,4*R)-3-(4-fluorophenyl)-7-hydroxyisochroman-4-yl)phenyl)piperidin-4-yl)methyl)piperazin-1-yl)-1-oxoisoindolin-2-yl)piperidine-2,6-dione as a brown oil. The brown oil was purified by preparative high-performance liquid chromatography. Condition: water(NH₃H₂O+NH₄HCO₃)-ACN. Column: Boston Prime C18 150*30 mm*5 um. Begin B: 60%, End B: 90%. Gradient Time (min) 7; 100% B hold Time (min) 0, Flow Rate (ml/min) 25. The pure fractions were collected and the solvent was evaporated under vacuum. The aqueous layer was lyophilized to dryness to give (S)-3-(5-(4-((1-(4-((3R,4S)-3-(4-fluorophenyl)-7-hydroxyisochroman-4-yl)phenyl)piperidin-4-yl)methyl)piperazin-1-yl)-1-oxoisoindolin-2-yl)piperidine-2,6-dione (13.5 mg, 17.8 μmol, 11%, 97.95% purity) as a white solid.

LCMS: calc. for C₄₄H₅₆FN₅O₅: 743.35, found: [M+H]⁺ 744.4.

HPLC: 97.95% purity at 220 nm.

¹H NMR (400 MHZ, DMSO-d₆) δ=10.95-10.91 (m, 1H), 9.29-9.26 (m, 1H), 7.54-7.49 (m, 1H), 7.09-7.02 (m, 4H), 6.98-6.92 (m, 2H), 6.80-6.75 (m, 1H), 6.60-6.53 (m, 6H), 5.10-4.99 (m, 3H), 4.94-4.87 (m, 1H), 4.37-4.28 (m, 1H), 4.25-4.15 (m, 1H), 4.04-3.97 (m, 1H), 3.54-3.47 (m, 2H), 3.29-3.23 (m, 5H), 2.91-2.84 (m, 1H), 2.74-2.66 (m, 1H), 2.63-2.53 (m, 5H), 2.36-2.30 (m, 1H), 2.22-2.14 (m, 2H), 2.00-1.91 (m, 1H), 1.79-1.70 (m, 2H), 1.68-1.58 (m, 1H), 1.24-1.11 (m, 2H).

### Example 75. Preparation of (I-35) (S)-3-(5-(4-((1-(4-((3S,4R)-3-(4-fluorophenyl)-7-hydroxyisochroman-4-yl)phenyl)piperidin-4-yl)methyl)piperazin-1-yl)-1-oxoisoindolin-2-yl)piperidine-2,6-dione

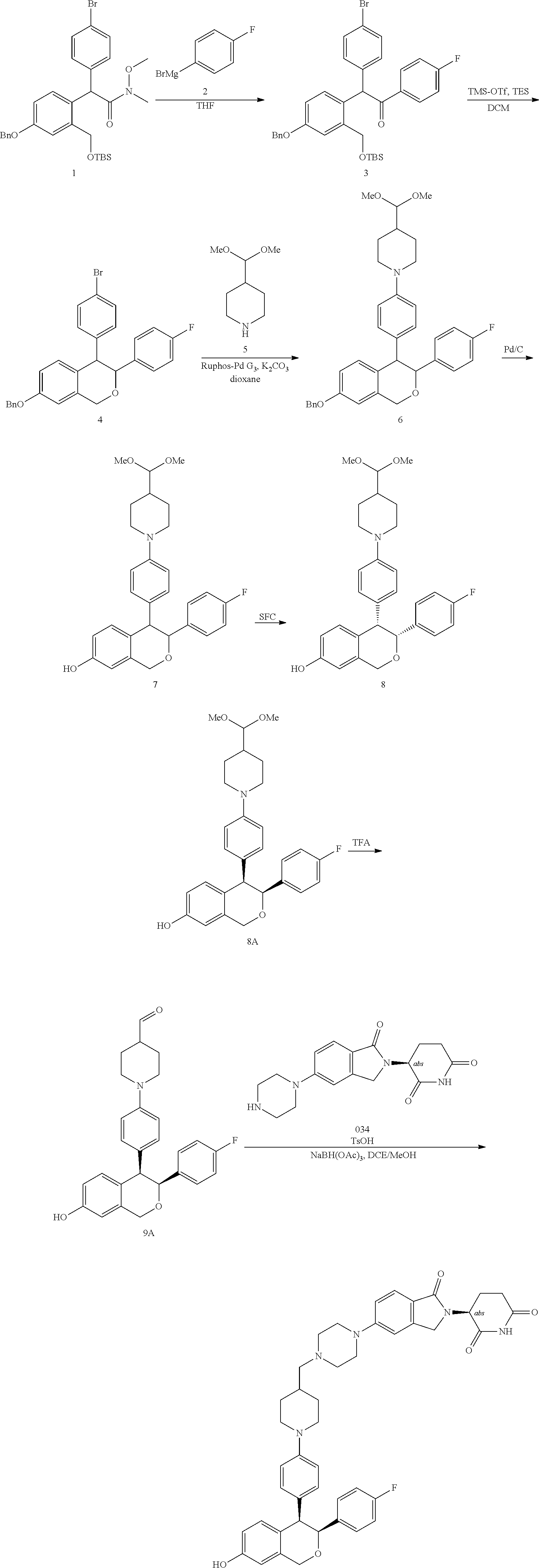

Step 1: To a solution of 1 (4 g, 1 Eq, 7 mmol) in THF (2 mL) was added 2 (7 g, 0.04 L, 0.8 molar, 5 Eq, 0.03 mol) at 15° C. Then the mixture was stirred at 15° C. for 12 hours under N₂. TLC showed that the new point was detected. NH₄CL (sat.aq.) (50 mL) was dropped into the mixture. Then the mixture was filtered and concentrated under vacuum to give the crude. Then the EtOAc (200 ml) was added into the mixture and extracted with EtOAc (3×200 mL). The organics players were dried by Na₂SO₄. The organics was concentrated under vacuum and purified by flash silica gel chromatography (ISCOR; 40 g SepaFlash® Silica Flash Column, Eluent of 0-10% Petroleum/Ethyl acetate ether gradient @ 60 mL/min) to give 3 (3 g, 70% yield) as yellow oil.

1H NMR (500 MHz, CHLOROFORM-d) δ=8.11-8.01 (m, 2H), 7.49-7.29 (m, 7H), 7.13-6.89 (m, 6H), 6.87-6.80 (m, 1H), 6.32 (s, 1H), 5.03 (s, 2H), 4.74-4.43 (m, 2H), 0.96-0.89 (m, 9H), 0.09 (d, J=4.4 Hz, 6H).

Step 2: To a solution of 3 (3.5 g, 1 Eq, 5.6 mmol) and Triethylsilane (3.3 g, 4.5 mL, 5 Eq, 28 mmol) in DCM (2 mL) was added Trimethylsilyl triflate (3.8 g, 3.1 mL, 3 Eq, 17 mmol) at −78° C. Then the mixture was warmed to 0° C. and stirred for 10 min. TLC showed that the new point was detected. NaHCO₃ (sat.aq.) (50 mL) was added into the mixture. Then the mixture was filtered and concentrated under vacuum to give the crude. Then the EtOAc (150 ml) was added into the mixture and extracted with EtOAc (3×150 mL). The organics players were dried by Na₂SO₄. The organics was concentrated under vacuum and purified by flash silica gel chromatography (ISCO®; 40 g SepaFlash® Silica Flash Column, Eluent of 0˜10% Petroleum/Ethyl acetate ether gradient @ 60 mL/min) to give 4 (2.1 g, 76% yield) as white solid.

1H NMR (400 MHZ, CHLOROFORM-d) δ=7.47-7.34 (m, 5H), 7.16 (d, J=8.3 Hz, 2H), 6.95-6.76 (m, 7H), 6.61 (d, J=8.5 Hz, 2H), 5.19-5.04 (m, 5H), 4.01 (d, J=3.0 Hz, 1H).

Step 3: To a solution of 4 (2.5 g, 1 Eq, 5.1 mmol) in 1,4-dioxane (60 mL) was added 5 (1.6 g, 2 Eq, 10 mmol) and Cs₂CO₃ (5.0 g, 3 Eq, 15 mmol) at 20° C. Then the (2-Dicyclohexylphosphino-2′,6′-diisopropoxy-1,1′-biphenyl) [2-(2′-amino-1,1′-biphenyl)]palladium (II) methanesulfonate (0.64 g, 0.15 Eq, 0.77 mmol) was added into the mixture at 20° C. The mixture was stirred at 90° C. for 6 hours under N₂. LCMS showed that the desired product was detected. The mixture was purified by flash silica gel chromatography (ISCO®; 40 g SepaFlash® Silica Flash Column, Eluent of 0-30% Petroleum/Ethyl acetate ether gradient @ 60 mL/min) to give 6 (1.5 g, 2.6 mmol, 52% yield) as yellow solid.

LC-MS (ESI⁺) m/z: 568.2 (M+H)⁺.

Step 4: A solution of 6 (1 g, 1 Eq, 2 mmol) and Pd/C (2 g, 10% Wt, 1 Eq, 2 mmol) in Ethyl acetate (50 mL) and then degassed and purged with H₂ for 3 times, and then the mixture was stirred at 15° C. for 12 hour under H₂ atmosphere. The reaction mixture was filtered and concentrated under reduced pressure to get the product. LCMS showed that the starting material was consumed, and the desired product was detected. The mixture was purified by flash silica gel chromatography (ISCO®; 20 g SepaFlash® Silica Flash Column, Eluent of 0-50% Petroleum/Ethyl acetate ether gradient @ 50 mL/min) to give 7 (600 mg, 70% yield) as white solid.

LC-MS (ESI⁺) m/z: 478.3 (M+H)⁺.

1H NMR (400 MHZ, DMSO-d₆) δ=9.28 (s, 1H), 7.09-7.02 (m, 2H), 6.99-6.91 (m, 2H), 6.77 (d. J=8.1 Hz, 1H), 6.60-6.53 (m, 6H), 5.06 (d, J=15.4 Hz, 1H), 5.00 (d, J=3.0 Hz,

1H), 4.90 (d, J=15.4 Hz, 1H), 4.04-3.97 (m, 2H), 3.52 (br d, J=12.2 Hz, 2H), 3.30 (s, 2H), 3.24 (s, 6H), 2.44-2.40 (m, 1H), 1.63 (br d, J=10.3 Hz, 2H), 1.31-1.20 (m, 2H)

Step 5: The 7 (1 g, 1 Eq, 2 mmol) was purified by SFC (condition: CO₂-EtOH (0.1% NH₃H₂O), column: DAICEL CHIRALPAK OJ (250 mm×30 mm, 10 um), Begin B: 30, End B: 30) to give 8 (300 mg, 30% yield) as white solid and 8A (300 mg, 30% yield) as white solid.

8: LC-MS (ESI⁺) m/z: 478.3 (M+H)⁺.

8A: LC-MS (ESI⁺) m/z: 478.2 (M+H)⁺.

Step 6: To a solution of 8A (300 mg, 1 Eq, 628 μmol) in DCM (30 mL) was added Trifluoroacetic acid (4 g, 3 mL, 6e+1 Eq, 0.04 mol) at 15° C. Then the mixture was stirred at 15° C. for 2 hours. LCMS showed that the desired product was detected. Then the mixture was filtered and concentrated under vacuum to give the 9A (300 mg, crude) as red oil.

LC-MS (ESI+) m/z: 450.5 (M+H+18)⁺

Step 7: To a solution of 9A (300 mg, 1 Eq, 695 μmol) in DCE (1 mL) and MeOH (1 mL) was added 034 (342 mg, 2 Eq. 1.04 mmol) and Sodium triacetoxyborohydride (737 mg, 5 Eq, 3.48 mmol) at 15° C. Then the mixture was stirred at 15° C. for 1 hour. LCMS showed that the desired product was detected. The mixture was filtered and concentrated under vacuum to give the crude. The residue was purified by prep-HPLC (FA condition: Column: Phenomenex Gemini NX 150×30 mm×5 um; Condition: water(NH₄HCO₃)-ACN, Begin B 45, End B 75, Gradient Time (min) 11, 100% B Hold Time (min) 2, Flow Rate (mL/min) 25.) to give I-35 (212.8 mg, 41% yield) as a white solid.

LCMS: calc. for C₄₄H₄₆FN₅O₅: 743.88, found: [M+H]⁺ 744.5.

HPLC: 100.00% purity at 220 nm

¹H NMR (400 MHZ, DMSO-d₆) δ=10.93 (br s, 1H), 9.29 (s, 1H), 7.51 (d, J=8.7 Hz, 1H), 7.10-7.01 (m, 4H), 7.00-6.92 (m, 2H), 6.77 (d, J=8.1 Hz, 1H), 6.61-6.52 (m, 6H), 5.09-4.98 (m, 3H), 4.90 (br d, J=15.3 Hz, 1H), 4.35-4.17 (m, 2H), 4.01 (d, J=2.5 Hz, 1H), 3.51 (br d, J=11.9 Hz, 2H), 3.27 (br s, 5H), 2.96-2.85 (m, 1H), 2.62-2.52 (m, 2H), 2.49-2.45 (m, 4H), 2.40-2.30 (m, 1H), 2.18 (br d, J=7.0 Hz, 2H), 2.00-1.92 (m, 1H), 1.74 (br d, J=11.7 Hz, 2H), 1.63 (br d, J=2.4 Hz, 1H), 1.22-1.09 (m, 2H).

### Example 76. Preparation of (I-226) (S)-3-(5-(4-((1-(4-((3S,4S)-7-hydroxy-3-isopropylisochroman-4-yl)phenyl)piperidin-4-yl)methyl)piperazin-1-yl)-1-oxoisoindolin-2-yl)piperidine-2,6-dione

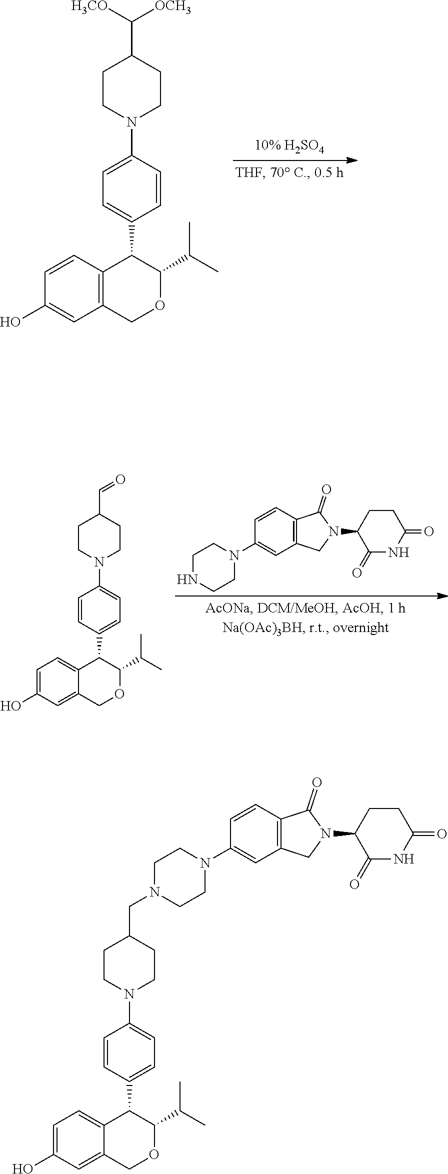 [SMILES: COC(OC)C1CCN(c2ccc([C@H]3c4ccc(O)cc4CO[C@H]3C(C)C)cc2)CC1 ; CC(C)[C@@H]1OCc2cc(O)ccc2[C@@H]1c1ccc(N2CCC(C=O)CC2)cc1 ; O=C1CC[C@H](N2Cc3cc(N4CCNCC4)ccc3C2=O)C(=O)N1 ; CC(C)[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1]

Step 1: A mixture of (3S,4S)-4-(4-(4-(dimethoxymethyl)piperidin-1-yl)phenyl)-3-isopropylisochroman-7-ol (85 mg, 1 Eq, 0.19 mmol) in THE (2.5 mL), then 10% H₂SO₄ (2.5 mL) was added to the mixture and stirred at 70° C. for 0.5 h. The reaction was adjusted to pH=8 with aq. NaHCO₃ at 0° C. Then the mixture was added H₂O (20 mL) and extracted with EtOAc (20 mL×2). The combined organic layers were washed with brine (20 mL), dried over anhydrous Na₂SO₄, filtered and concentrated to dryness in vacuo to give 1-(4-((3S,4S)-7-hydroxy-3-isopropylisochroman-4-yl)phenyl)piperidine-4-carbaldehyde (75 mg, 100%, 98.970% Purity) as a yellow solid. LC-MS (ESI+) m/z: 380.2 (M+H)⁺.

Step 2: A mixture of 1-(4-((3S,4S)-7-hydroxy-3-isopropylisochroman-4-yl)phenyl)piperidine-4-carbaldehyde (75 mg, 1 Eq, 0.20 mmol), (S)-3-(1-oxo-5-(piperazin-1-yl)isoindolin-2-yl)piperidine-2,6-dione benzenesulfonate (0.12 g, 1.3 Eq, 0.25 mmol), sodium acetate (80 mg, 5 Eq, 0.98 mmol) and AcOH (35 mg, 34 μL, 3 Eq, 0.59 mmol) in DCM (3 mL) and MeOH (3 mL) was stirred at 25° C. for 1 b, then sodium triacetoxyborohydride (83 mg, 2 Eq, 0.39 mmol) was added to the mixture and was stirred at 25° C. for 16 h. Then the mixture was added H₂O (20 mL) and extracted with EtOAc (20 mL×2). The combined organic layers were washed with brine (20 mL), dried over anhydrous Na₂SO₄, filtered and concentrated to dryness in vacuo to give a residue. The residue was purified by prep-HPLC (column: Welch Xtimate C18 150*30 mm*5 um; mobile phase: [water (FA)-ACN]; B % 10%-50%, 8 min) to give (S)-3-(5-(4-((1-(4-((3S,4S)-7-hydroxy-3-isopropylisochroman-4-yl)phenyl)piperidin-4-yl)methyl)piperazin-1-yl)-1-oxoisoindolin-2-yl)piperidine-2,6-dione (59.2 mg, 44%, 99.519% Purity) as a white solid. LC-MS (ESI+) m/z: 692.4 (M+H)⁺.

LCMS: calc. for C₄₁H₄₉N₅O₅:691.37, found: [M+H]⁺ 692.4.

HPLC: 99.519% purity at 220 nm.

¹HNMR (400 MHz, DMSO-d₆) δ=10.95 (s, 1H), 9.38-9.04 (m, 1H), 8.17 (s, 1H), 7.53 (d, J=8.7 Hz, 1H), 7.13-7.04 (m, 4H), 6.77 (dd, J=5.8, 8.4 Hz, 3H), 6.50 (dd, J=2.3, 8.2 Hz, 1H), 6.44 (d, J=2.1 Hz, 1H), 5.05 (dd, J=5.1, 13.3 Hz, 1H), 4.89 (d, J=15.4 Hz, 1H), 4.67 (br d, J=15.0 Hz, 1H), 4.38-4.30 (m, 1H), 4.26-4.17 (m, 1H), 3.77 (d, J=1.9 Hz, 1H), 3.59 (br d, J=9.9 Hz, 2H), 3.45-3.32 (m, 4H), 3.24 (br dd, J=2.7, 9.8 Hz, 2H), 2.96-2.85 (m, 1H), 2.64-2.52 (m, 4H), 2.50-2.47 (m, 2H), 2.43-2.30 (m, 1H), 2.22 (br d, J=7.2 Hz, 2H), 2.01-1.92 (m, 1H), 1.80 (br d, J=11.2 Hz, 2H), 1.67 (br s, 1H), 1.30-1.14 (m, 3H), 0.93-0.82 (m, 6H).

### Example 77. Preparation of (I-268) (S)-3-(5-(4-((1-(4-((1′R,4′S)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione

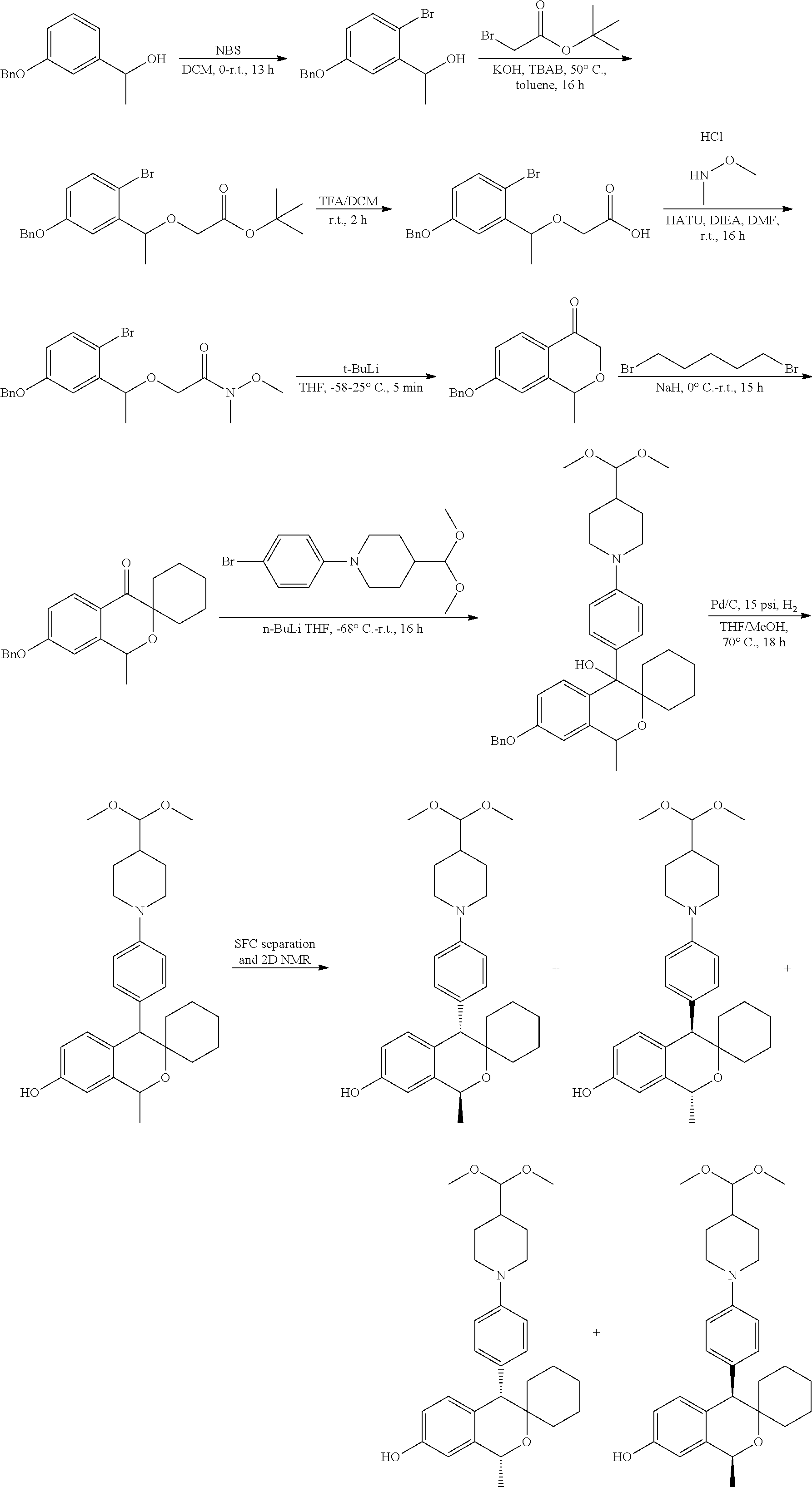

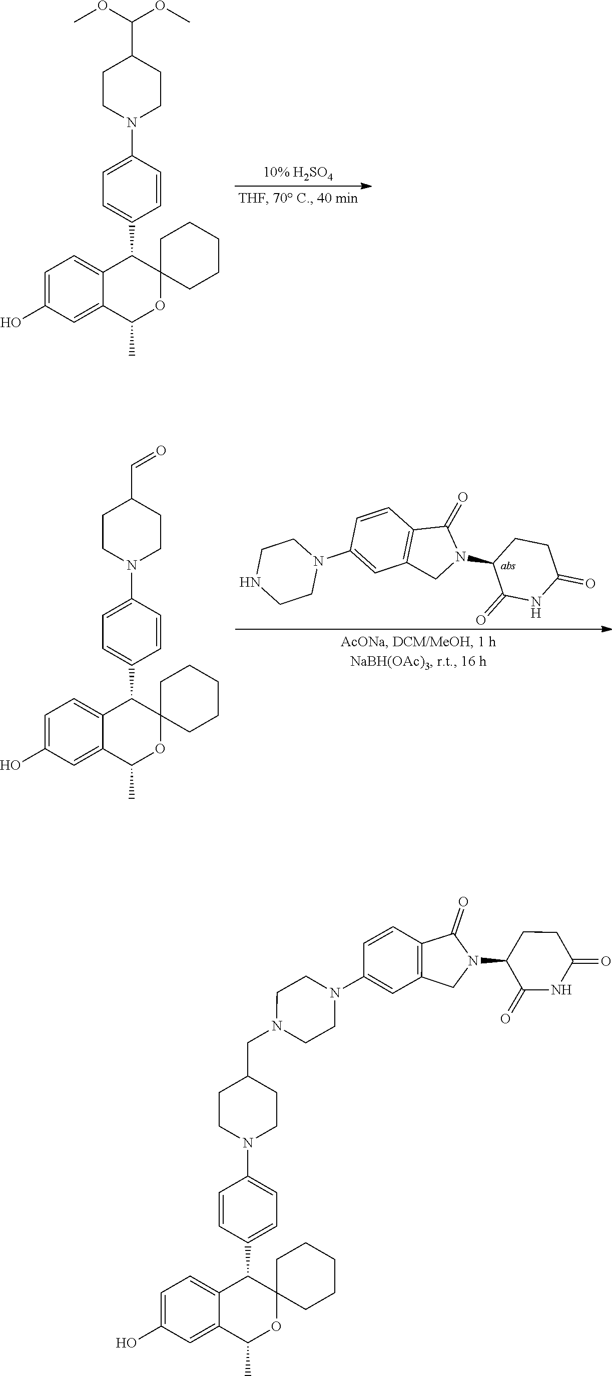 [SMILES: COC(OC)C1CCN(c2ccc([C@H]3c4ccc(O)cc4[C@@H](C)OC34CCCCC4)cc2)CC1 ; C[C@H]1OC2(CCCCC2)[C@@H](c2ccc(N3CCC(C=O)CC3)cc2)c2ccc(O)cc21 ; O=C1CC[C@H](N2Cc3cc(N4CCNCC4)ccc3C2=O)C(=O)N1 ; C[C@H]1OC2(CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21]

Step 1: To a mixture of 1-(3-(benzyloxy)phenyl) ethan-1-ol (8 g, 1 Eq, 35 mmol) and 1-bromopyrrolidine-2,5-dione (6.9 g, 1.1 Eq, 39 mmol) in DCM (150 mL) at 0° C. for 1 hour, then the mixture was stirred at 25° C. for 12 h. TLC (petroleum ether:ethyl acetate=5:1, R_{f}=0.7, UV) showed one main new spot was observed. The reaction was diluted with water (200 mL) and extracted with ethyl acetate (200 mL*2). The organic layer was washed with brine (200 mL) and dried over Na₂SO₄ and concentrated in vacuo to give a white solid. The white solid was subjected to column chromatography over silica gel (gradient elution: 0-15% EtOAc). The desired fractions were collected, and concentrated to dryness in vacuo to give 1-(5-(benzyloxy)-2-bromophenyl) ethan-1-ol (8.9 g, 29 mmol, 83% yield) as a white solid which confirmed by HNMR.

Step 2: A mixture of tetrabutylammonium bromide (1 g, 0.1 Eq, 3.2 mmol), potassium hydroxide (4.5 g, 40% Wt, 1 Eq, 32 mmol), tert-butyl 2-bromoacetate (7.5 g, 1.2 Eq, 39 mmol) 1-(5-(benzyloxy)-2-bromophenyl) ethan-1-ol (9.9 g, 1 Eq, 32 mmol) in toluene (150 mL) was degassed and purged with N₂ for 3 times, and then the mixture was stirred at 50° C. for 16 h under N₂ atmosphere. TLC (petroleum ether:ethyl acetate=5:1, R_{f}=0.5 UV) showed one main new spot was observed. The reaction was diluted with water (200 mL) and extracted with ethyl acetate (250 mL*2). The organic layer was washed with brine (200 mL) and dried over anhydrous Na₂SO₄ and concentrated in vacuo to give a white solid. The white solid was subjected to column chromatography over silica gel (gradient elution: 0-30% EtOAc). The desired fractions were collected, and concentrated to dryness in vacuo to give tert-butyl 2-(1-(5-(benzyloxy)-2-bromophenyl) ethoxy)acetate (7.4 g, 18 mmol, 54% yield) as a white solid which confirmed by HNMR.

Step 3: To a mixture of tert-butyl 2-(1-(5-(benzyloxy)-2-bromophenyl) ethoxy)acetate (10.4 g, 1 Eq, 24.7 mmol) in DCM (150 mL) and TFA (30 mL), then the mixture was stirred at 25° C. for 2 h. TLC (petroleum ether:ethyl acetate=5:1, R_{f}=0.3 UV) showed one main new spot was observed. The reaction was quenched with water (200 mL) and extracted with ethyl acetate (250 mL*2). The organic layer was washed with brine (200 mL) and dried over anhydrous Na₂SO₄ and concentrated in vacuo to give a white solid. The white solid was subjected to column chromatography over silica gel (gradient elution: 0-50% EtOAc in petroleum ether). The desired fractions were collected, and concentrated to dryness in vacuo to give 2-(1-(5-(benzyloxy)-2-bromophenyl) ethoxy) acetic acid (5 g, 0.01 mol, 60% yield) as a white solid which confirmed by HNMR.

Step 4: To a solution of o,n-dimethyl-hydroxylamine hcl (1.3 g, 1 Eq, 14 mmol) and 2-(1-(5-(benzyloxy)-2-bromophenyl) ethoxy) acetic acid (5 g, 1 Eq, 14 mmol) in DMF (80 mL) was added HATU (7.8 g, 1.5 Eq, 21 mmol) and DIEA (5.3 g, 7.1 mL, 3 Eq, 41 mmol). The mixture was stirred at 25° C. for 16 hour. TLC (petroleum ether:ethyl acetate=5:1, Rf=0.5 UV) showed one main new spot was observed. The reaction was diluted with water (200 mL) and extracted with ethyl acetate (200 mL*2). The organic layer was washed with brine (200 mL) and dried over anhydrous Na₂SO₄ and concentrated in vacuo to give a white solid. The white solid was subjected to column chromatography over silica gel (gradient elution: 0-50% EtOAc). The desired fractions were collected, and concentrated to dryness in vacuo to give 2-(1-(5-(benzyloxy)-2-bromophenyl) ethoxy)-N-methoxy-N-methylacetamide (4 g, 0.01 mol, 70% yield) as a white solid.

Step 5: A solution of 2-((5-(benzyloxy)-2-bromobenzyl)oxy)-N-methoxy-N-methylacetamide (4 g, 1 Eq, 10.1 mmol) in THF (100 mL) was stirred at −78° C. for 10 min under N₂ atmosphere, then added tert-butyllithium (1.5 g, 17.9 mL, 1.3 molar, 2.3 Eq, 23.3 mmol) at −78° C. The reaction was stirred at −78° C. to 25° C. for 5 min under N₂ atmosphere. TLC (petroleum ether:ethyl acetate=5:1, UV) showed one main new spot was observed. The reaction was quenched with aq. NH₄Cl (100 mL) and extracted with ethyl acetate (100 mL*3). The organic layer was washed with brine (100 mL) and dried over anhydrous Na₂SO₄ and concentrated in vacuo to give crude product. The black oil was subjected to column chromatography over silica gel (gradient elution: 0-20% EtOAc). The desired fractions were collected, and concentrated to dryness in vacuo to give 7-(benzyloxy)isochroman-4-one (2.3 g, 9 mmol, 89% yield) as a yellow solid.

Step 6: To a cooled stirred (ice-salt) suspension of sodium hydride (148 mg, 0.12 mL, 60% Wt, 3 Eq, 3.69 mmol) in DMF (8 mL) was added dropwise a solution of 7-(benzyloxy)-1-methylisochroman-4-one (330 mg, 1 Eq, 1.23 mmol), then stirred at 0° C. for 2 hour. After this time, 1,5-dibromo-pentane (424 mg, 251 μL, 1.5 Eq, 1.84 mmol) was added to the reaction and stirred at 25° C. for 16 hour. TLC (petroleum ether:ethyl acetate=10:1, R_{f}=0.4) showed new spots was observed. The reaction was quenched with water (100 mL) and extracted with ethyl acetate (150 mL*2). The organic layer was washed with brine (100 mL) and dried over anhydrous Na₂SO₄ and concentrated in vacuo to give a white solid. The white solid was subjected to column chromatography over silica gel (gradient elution: 0-50% EtOAc). The desired fractions were collected and concentrated to dryness in vacuo to give 7′-(benzyloxy)-1′-methylspiro[cyclohexane-1,3′-isochroman]-4′-one (90 mg, 0.27 mmol, 22% yield) as a yellow oil. LC-MS (ESI+) m/z: 337.1 (M+H)⁺.

Step 7: To a mixture of 1-(4-bromophenyl)-4-(dimethoxymethyl)piperidine (294 mg, 3.5 Eq, 936 μmol) in THF (6 mL) at r.t., and the mixture was degassed and purged with N₂ for 10 minutes. Then the mixture was stirred until it reached −78° C. n-Butyllithium (51.4 mg, 321 μL, 2.5 molar, 3 Eq, 803 μmol) was added to the mixture and stirred at −78° C. for 1 hour, then 7′-(benzyloxy)-1′-methylspiro[cyclohexane-1,3′-isochroman]-4′-one (90 mg, 1 Eq, 268 μmol) was added to the mixture at −78° C. and stirred at 25° C. for 16 hour. TLC (petroleum ether:ethyl acetate=5:1, R_{f}=0.4 UV) showed one main new spot was observed. The reaction was quenched with NH₄Cl (100 mL) and extracted with ethyl acetate (100 mL*2). The organic layer was washed with brine (100 mL) and dried over anhydrous Na₂SO₄ and concentrated in vacuo to give a yellow oil. The yellow oil was subjected to column chromatography over silica gel (gradient elution: 0-60% EtOAc). The desired fractions were collected, and concentrated to dryness in vacuo to give 7′-(benzyloxy)-4′-(4-(4-(dimethoxymethyl)piperidin-1-yl)phenyl)-1′-methylspiro[cyclohexane-1,3′-isochroman]-4′-ol (80 mg, 0.14 mmol, 52% yield) as a yellow oil.

Step 8: A mixture of 7′-(benzyloxy)-4′-(4-(4-(dimethoxymethyl)piperidin-1-yl)phenyl)-1′-methylspiro[cyclohexane-1,3′-isochroman]-4′-ol (60 mg, 1 Eq. 0.10 mmol), Pd/C (150 mg, 10% Wt, 1.3 Eq, 141 μmol) in MeOH (15 mL) and THF (15 mL) was degassed and purged with H₂ for 3 times, and then the mixture was stirred at 70° C. for 18 h under H₂ atmosphere (15 psi). TLC (petroleum ether:ethyl acetate=5:1, R_{f}=0.3 UV) showed one main new spot was observed. The reaction mixture was filtered and concentrated under reduced pressure to give residue. The residue was concentrated under reduced pressure to give 4′-(4-(4-(dimethoxymethyl)piperidin-1-yl)phenyl)-1′-methylspiro[cyclohexane-1,3′-isochroman]-7′-ol (90 mg, 0.11 mmol) as a yellow oil.

Step 9: The reaction mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um); mobile phase: [CO₂-i-PrOH (0.1% NH₃H₂O)]; B %: 40%-40%) to give (1′S,4′S)-4′-(4-(4-(dimethoxymethyl)piperidin-1-yl)phenyl)-1′-methylspiro[cyclohexane-1,3′-isochroman]-7′-ol, (20 mg) (1′R,4′R)-4′-(4-(4-(dimethoxymethyl)piperidin-1-yl)phenyl)-1′-methylspiro[cyclohexane-1,3′-isochroman]-7′-ol, (20 mg) (1′R,4′S)-4′-(4-(4-(dimethoxymethyl)piperidin-1-yl)phenyl)-1′-methylspiro[cyclohexane-1,3′-isochroman]-7-ol (20 mg) and (1′S,4′R)-4′-(4-(4-(dimethoxymethyl)piperidin-1-yl)phenyl)-1′-methylspiro[cyclohexane-1,3′-isochroman]-7′-ol (20 mg) as yellow solid.

Step 10: To a solution of (1′R,4′S)-4′-(4-(4-(dimethoxymethyl)piperidin-1-yl)phenyl)-1′-methylspiro[cyclohexane-1,3′-isochroman]-7′-ol (20 mg, 1 Eq, 43 μmol) in 10% H₂SO₄ (3 mL) and THF (3 mL) was stirred at 70° C. for 40 min. TLC (petroleum ether:ethyl acetate=3:1, R_{f}=0.4, UV) showed a new spot was desired. The reaction was adjust to pH=˜8 with saturated aq. NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1′R,4′S)-7′-hydroxy-1′-methylspiro[cyclohexane-1,3′-isochroman]-4′-yl)phenyl)piperidine-4-carbaldehyde (10 mg) as a yellow solid. LC-MS (ESI⁺) m/z: 420.3 (M+H)⁺.

Step 11: To a solution of 1-(4-((1′R,4′S)-7′-hydroxy-1′-methylspiro[cyclohexane-1,3′-isochroman]-4′-yl)phenyl)piperidine-4-carbaldehyde (10 mg, 1 Eq), (S)-3-(1-oxo-5-(piperazin-1-yl)isoindolin-2-yl)piperidine-2,6-dione benzenesulfonate (12 mg, 1 Eq, 24 μmol) in DCM (4 mL) and MeOH (4 mL) was added sodium acetate (6 mg, 3 Eq, 72 μmol) was stirred at 25° C. for 1 h, Then sodium triacetoxyborohydride (10 mg, 7.1 μL, 2 Eq, 48 μmol) and acetic acid (4 mg, 4 μL, 3 Eq, 72 μmol) was added. The mixture was stirred at 25° C. for 16 hour. The reaction was diluted with H₂O (50 mL) and extracted with ethyl acetate (50 mL*2). The organic layer was washed with brine (30 mL) and dried over anhydrous Na₂SO₄ and concentrated in vacuo to give a yellow oil. The residue was purified by prep-HPLC (column: Welch Xtimate C18 150*30 mm, mobile phase: [water (FA)-ACN]; B % 20%-50%, 6 min) to give (S)-3-(5-(4-((1-(4-((1′R,4′S)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione (3.5 mg, 4.7 μmol, 10% yield, 99.27% purity) was obtained as a white solid. LC-MS (ESI⁺) m/z: 732.3 (M+H)⁺.

LCMS: calc. for C₄₄H₅₃N₅O₅: 731.94, found: [M+H]⁺ 732.3.

HPLC: 99.273% purity at 220 nm.

¹H NMR (400 MHz, DMSO-d₆) δ=10.94 (s, 1H), 9.20-9.11 (m, 1H), 8.21 (s, 0.264H), 7.52 (d, J=8.7 Hz, 1H), 7.07 (s, 2H), 6.96 (br d, J=6.4 Hz, 2H), 6.77 (d, J=8.8 Hz, 2H), 6.70 (d, J=8.2 Hz, 1H), 6.56 (s, 1H), 6.51 (dd, J=2.3, 8.3 Hz, 1H), 5.05 (dd, J=5.2, 13.3 Hz, 1H), 4.73 (br d, J=5.8 Hz, 1H), 4.38-4.29 (m, 1H), 4.26-4.16 (m, 1H), 3.65-3.54 (m, 2H), 3.48 (s, 1H), 3.40-3.33 (m, 8H), 2.97-2.84 (m, 1H), 2.58 (br s, 3H), 2.41-2.31 (m, 1H), 2.22 (br d, J=7.2 Hz, 2H), 2.02-1.92 (m, 1H), 1.81 (br s, 3H), 1.67 (br s, 1H), 1.53 (d, J=6.4 Hz, 3H), 1.46 (br d, J=0.8 Hz, 4H), 1.32 (br s, 2H), 1.21 (brs, 3H), 1.10 (br s, 1H), 0.99 (br s, 1H).

### Example 78. Preparation of (I-83) (S)-3-(5-(4-((1-(4-((1′S,4′R)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione

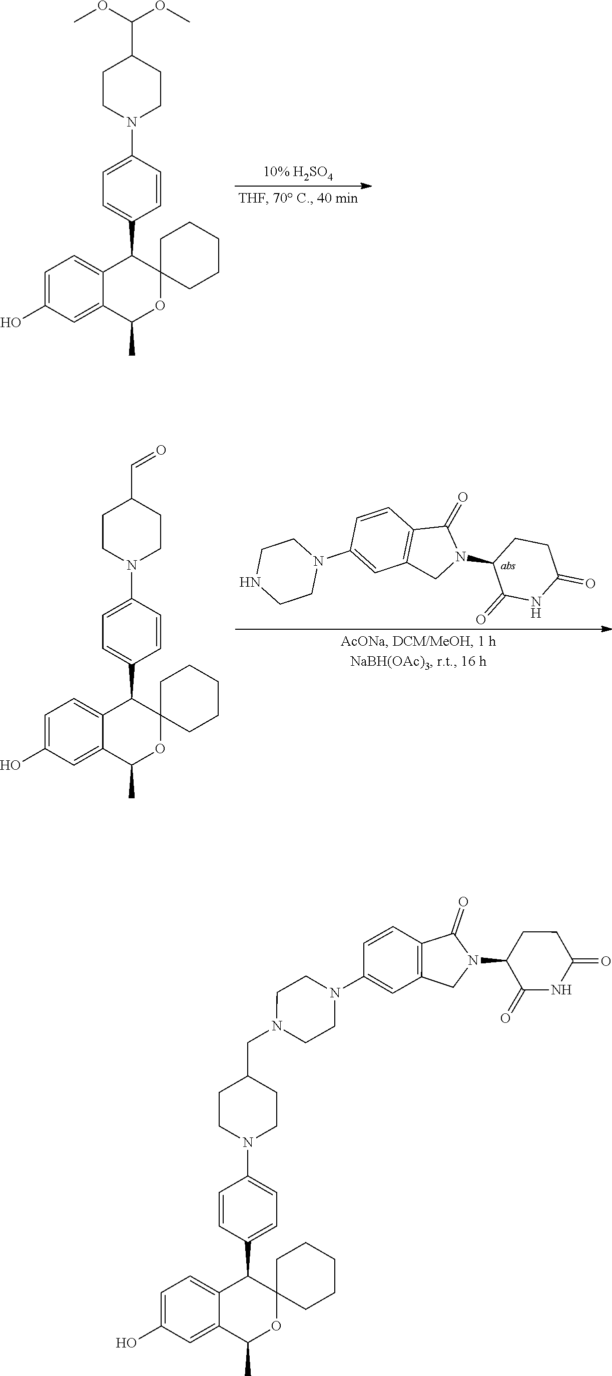 [SMILES: COC(OC)C1CCN(c2ccc([C@@H]3c4ccc(O)cc4[C@H](C)OC34CCCCC4)cc2)CC1 ; C[C@@H]1OC2(CCCCC2)[C@H](c2ccc(N3CCC(C=O)CC3)cc2)c2ccc(O)cc21 ; O=C1CC[C@H](N2Cc3cc(N4CCNCC4)ccc3C2=O)C(=O)N1 ; C[C@@H]1OC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21]

Step 1: To a solution of (1′S,4′R)-4′-(4-(4-(dimethoxymethyl)piperidin-1-yl)phenyl)-1′-methylspiro[cyclohexane-1,3′-isochroman]-7′-ol (20 mg, 1 Eq) in 10% H₂SO₄ (3 mL) and THE (3 mL) was stirred at 70° C. for 40 min. TLC (petroleum ether:ethyl acetate=3:1, R_{f}=0.4, UV) showed a new spot was desired. The reaction was adjust to pH=˜8 with saturated aq. NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1′S,4′R)-7-hydroxy-1′-methylspiro[cyclohexane-1,3′-isochroman]-4′-yl)phenyl)piperidine-4-carbaldehyde (10 mg, 24 μmol) as a yellow solid. LC-MS (ESI+) m/z: 438.2 (M+H₂O)⁺.

Step 2: To a solution of 1-(4-(1′S,4′R)-7′-hydroxy-1′-methylspiro[cyclohexane-1,3′-isochroman]-4′-yl)phenyl)piperidine-4-carbaldehyde (10 mg, 1 Eq, 24 μmol), (S)-3-(1-oxo-5-(piperazin-1-yl)isoindolin-2-yl)piperidine-2,6-dione benzenesulfonate (12 mg, 1 Eq, 24 μmol) in DCM (4 mL) and MeOH (4 mL) was added Sodium acetate (6 mg, 3 Eq, 72 μmol) was stirred at 25° C. for 1 h, Then Sodium triacetoxyborohydride (10 mg, 7.1 μL, 2 Eq, 48 μmol) and acetic acid (4 mg, 4 μL, 3 Eq, 72 μmol) was added. The mixture was stirred at 25° C. for 16 h. The reaction was quenched with H₂O (50 mL) and extracted with ethyl acetate (50 mL*2). The organic layer was washed with brine (30 mL) and dried over anhydrous Na₂SO₄ and concentrated in vacuo to give a yellow oil. The residue was purified by prep-HPLC (column: Welch Xtimate C18 150*30 mm; mobile phase: [water (FA)-ACN]; B % 20%-50%, 6 min) to give (S)-3-(5-(4-((1-(4-((1′S,4′R)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione (3.4 mg, 4.6 μmol, 19%, 100% purity) was obtained as a white solid. LC-MS (ESI⁺) m/z: 732.3 (M+H)⁺.

LCMS: calc. for C₄₄H₅₃N₅O₅: 731.94, found: [M+H]⁺ 732.3.

HPLC: 100% purity at 220 nm.

¹H NMR (400 MHZ, DMSO-d₆) δ=10.94 (s, 1H), 9.15 (br d, J=5.0 Hz, 1H), 8.19 (s, 0.267H), 7.52 (d, J=8.7 Hz, 1H), 7.07 (s, 2H), 6.95 (br s, 2H), 6.77 (d, J=8.8 Hz, 2H), 6.70 (d, J=8.2 Hz, 1H), 6.56 (s, 1H), 6.51 (dd, J=2.3, 8.2 Hz, 1H), 5.05 (dd, J=5.0, 13.1 Hz, 1H), 4.73 (br d, J=6.4 Hz, 1H), 4.38-4.29 (m, 1H), 4.26-4.15 (m, 1H), 3.64-3.54 (m, 2H), 3.48 (s, 1H), 3.33 (br s, 8H), 2.90 (br s, 1H), 2.58 (br s, 3H), 2.37 (br d, J=12.9 Hz, 1H), 2.22 (br d, J=7.0 Hz, 2H), 1.97 (br d, J=5.6 Hz, 1H), 1.79 (br d, J=11.3 Hz, 3H), 1.66 (br s, 1H), 1.53 (d, J=6.4 Hz, 3H), 1.47 (br s, 4H), 1.32 (br s, 2H), 1.21 (br s, 3H), 1.10 (br s, 1H), 0.99 (br s, 1H).

### Example 79. Preparation of (I-269) (S)-3-(5-(4-((1-(4-((1R,4R)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione

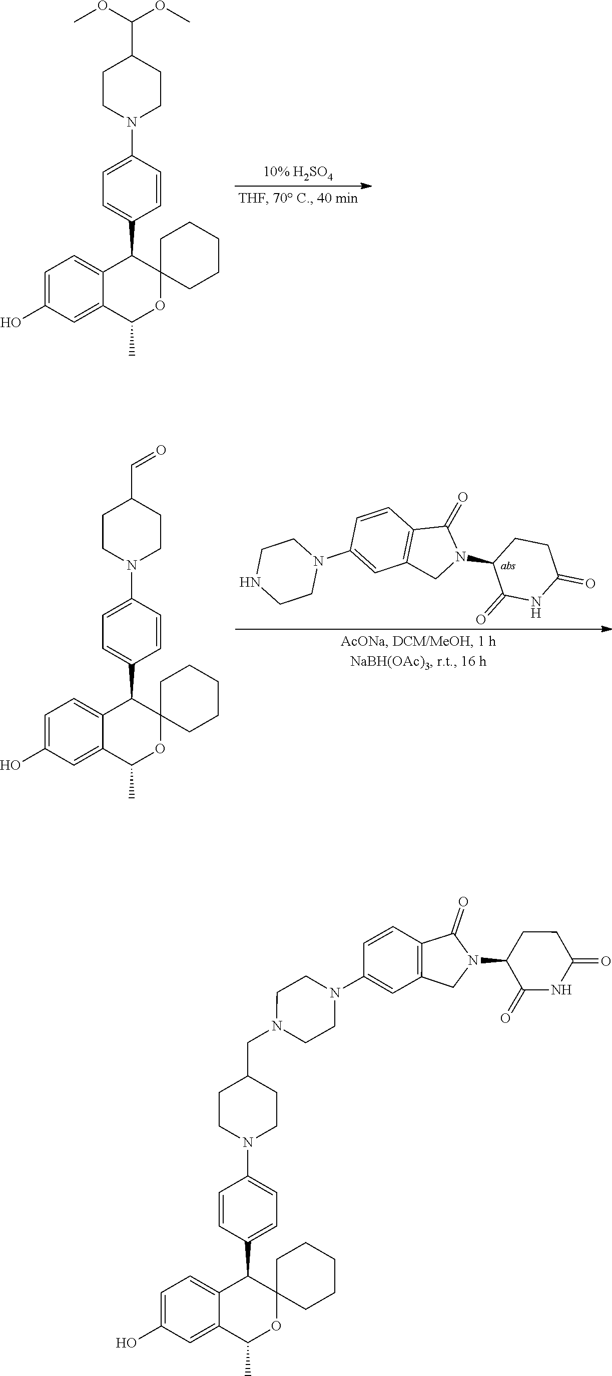 [SMILES: COC(OC)C1CCN(c2ccc([C@@H]3c4ccc(O)cc4[C@@H](C)OC34CCCCC4)cc2)CC1 ; C[C@H]1OC2(CCCCC2)[C@H](c2ccc(N3CCC(C=O)CC3)cc2)c2ccc(O)cc21 ; O=C1CC[C@H](N2Cc3cc(N4CCNCC4)ccc3C2=O)C(=O)N1 ; C[C@H]1OC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21]

Step 1: To a solution of (1R,4R)-4′-(4-(4-(dimethoxymethyl)piperidin-1-yl)phenyl)-1′-methylspiro[cyclohexane-1,3′-isochroman]-7′-ol (20 mg, 1 Eq, 86 μmol) in 10% H₂SO₄ (3 mL) and THF (3 mL) was stirred at 70° C. for 40 min. TLC (petroleum ether:ethyl acetate=3:1, R_{f}=0.4, UV) showed a new spot was desired. The reaction was adjusted to pH=˜8 with saturated aq. NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1R,4R)-7′-hydroxy-1′-methylspiro[cyclohexane-1,3′-isochroman]-4′-yl)phenyl)piperidine-4-carbaldehyde (10 mg) as a yellow solid. LC-MS (ESI+) m/z: 438.2 (M+H)⁺.

Step 2: To a solution of 1-(4-((1R,4R)-7′-hydroxy-1′-methylspiro[cyclohexane-1,3′-isochroman]-4′-yl)phenyl)piperidine-4-carbaldehyde (10 mg, 1 Eq), (S)-3-(1-oxo-5-(piperazin-1-yl)isoindolin-2-yl)piperidine-2,6-dione benzenesulfonate (12 mg, 1 Eq, 24 μmol) in DCM (4 mL) and MeOH (4 mL) was added Sodium acetate (6 mg, 3 Eq, 72 μmol) was stirred at 25° C. for 1 h, Then Sodium triacetoxyborohydride (10 mg, 7.1 μL, 2 Eq, 48 μmol) and acetic acid (4 mg, 4 μL, 3 Eq, 72 μmol) was added. The mixture was stirred at 25° C. for 16 hours. The reaction was quenched with H₂O (50 mL) and extracted with ethyl acetate (50 mL*2). The organic layer was washed with brine (30 mL) and dried over anhydrous Na₂SO₄ and concentrated in vacuo to give a yellow oil. The residue was purified by prep-HPLC (column: Welch Xtimate C18 150*30 mm; mobile phase: [water (FA)-ACN]; B % 20%-50%, 6 min) to give (S)-3-(5-(4-((1-(4-((1R,4R)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione (3.7 mg, 5 μmol, 7.1%, 99.85% purity) was obtained as a white solid. LC-MS (ESI⁺) m/z: 732.3 (M+H)⁺.

LCMS: calc. for C₄₄H₅₃N₅O₅:731.94, found: [M+H]⁺ 732.3.

HPLC: 99.85% purity at 220 nm.

¹H NMR (400 MHZ, DMSO-d₆) δ=10.95 (s, 1H), 9.16 (s, 1H), 8.16 (s, 0.203H), 7.53 (d, J=8.7 Hz, 1H), 7.08 (s, 2H), 6.92 (br s, 2H), 6.89-6.83 (m, 2H), 6.55 (s, 1H), 6.50 (s, 2H), 5.05 (dd, J=5.1, 13.3 Hz, 1H), 4.80 (br d, J=6.2 Hz, 1H), 4.38-4.30 (m, 1H), 4.27-4.16 (m, 1H), 3.79 (s, 1H), 3.68 (br d, J=11.2 Hz, 2H), 3.42-3.33 (m, 8H), 2.89 (br d, J=12.0 Hz, 1H), 2.63 (br d, J=13.2 Hz, 3H), 2.37 (br dd, J=4.7, 13.3 Hz, 1H), 2.24 (br d, J=7.0 Hz, 2H), 1.97 (br d, J=5.2 Hz, 1H), 1.84 (br d. J=10.8 Hz, 2H), 1.71 (br s, 3H), 1.55 (br s, 2H), 1.43 (br d, J=6.4 Hz, 6H), 1.23 (br d, J=8.9 Hz, 3H), 1.04-0.84 (m, 2H).

### Example 80. Preparation of (I-270) (S)-3-(5-(4-((1-(4-((1′S,4′S)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione

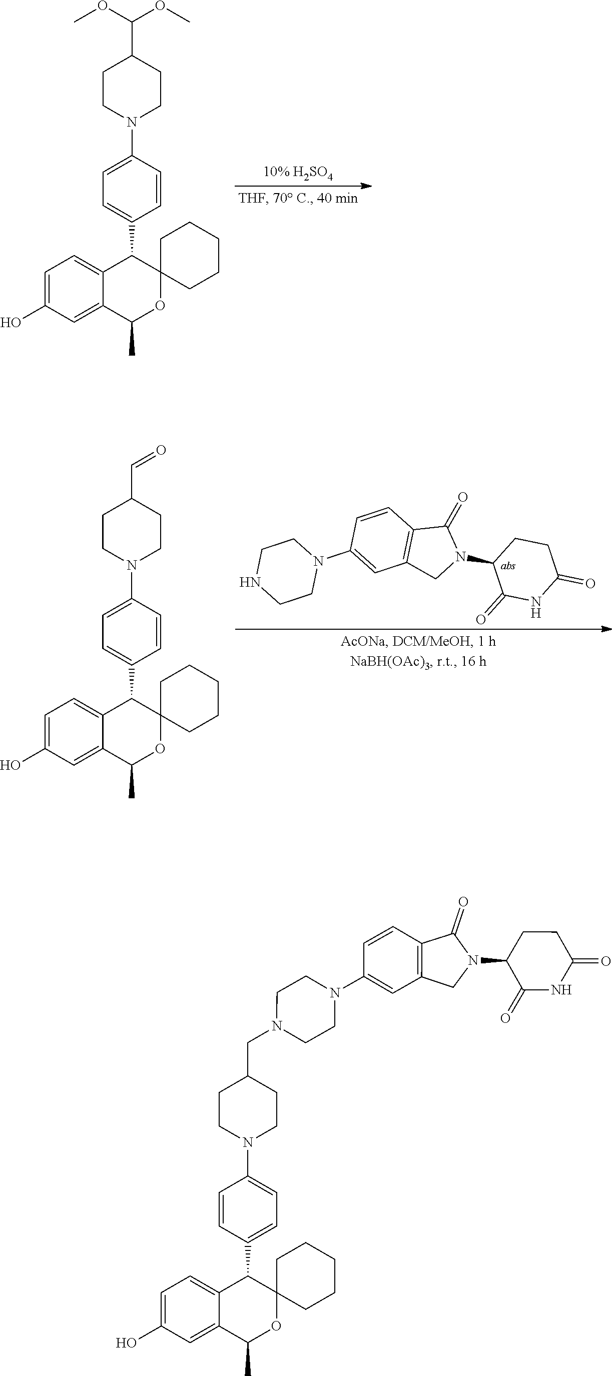 [SMILES: COC(OC)C1CCN(c2ccc([C@H]3c4ccc(O)cc4[C@H](C)OC34CCCCC4)cc2)CC1 ; C[C@@H]1OC2(CCCCC2)[C@@H](c2ccc(N3CCC(C=O)CC3)cc2)c2ccc(O)cc21 ; O=C1CC[C@H](N2Cc3cc(N4CCNCC4)ccc3C2=O)C(=O)N1 ; C[C@@H]1OC2(CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21]

Step 1: To a solution of (1S,4S)-4′-(4-(4-(dimethoxymethyl)piperidin-1-yl)phenyl)-1′-methylspiro[cyclohexane-1,3′-isochroman]-7′-ol (20 mg, 1 Eq, 64 μmol) in 10% H₂SO₄ (3 mL) and THF (3 mL) was stirred at 70° C. for 40 min. TLC (petroleum ether:ethyl acetate=3:1, R_{f}=0.4, UV) showed a new spot was desired. The reaction was adjusted to pH=˜8 with saturated aq. NaHCO₃ (15 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuo to give 1-(4-((1S,4S)-7′-hydroxy-1′-methylspiro[cyclohexane-1,3′-isochroman]-4′-yl)phenyl)piperidine-4-carbaldehyde (10 mg) as a yellow solid. LC-MS (ESI+) m/z: 438.1 (M+H₂O)⁺.

Step 2: To a solution of 1-(4-((1S,4S)-7-hydroxy-1′-methylspiro[cyclohexane-1,3′-isochroman]-4′-yl)phenyl)piperidine-4-carbaldehyde (10 mg, 1 Eq), (S)-3-(1-oxo-5-(piperazin-1-yl)isoindolin-2-yl)piperidine-2,6-dione benzenesulfonate (12 mg, 1 Eq, 24 μmol) in DCM (4 mL) and MeOH (4 mL) was added sodium acetate (6 mg, 3 Eq, 72 μmol) was stirred at 25° C. for 1 h, Then sodium triacetoxyborohydride (10 mg, 7.1 μL, 2 Eq, 48 μmol) and acetic acid (4 mg, 4 μL, 3 Eq, 72 μmol) was added. The mixture was stirred at 30° C. for 16 hours. The reaction was quenched with H₂O (50 mL) and extracted with ethyl acetate (50 mL*2). The organic layer was washed with brine (30 mL) and dried over anhydrous Na₂SO₄ and concentrated in vacuo to give a yellow oil. The residue was purified by prep-HPLC (column: Welch Xtimate C18 150*30 mm; mobile phase: [water (FA)-ACN]; B % 20%-50%, 6 min) to give (S)-3-(5-(4-((1-(4-((1S,4S)-7′-hydroxy-1′-methylspiro[cyclohexane-1,3′-isochroman]-4′-yl)phenyl)piperidin-4-yl)methyl)piperazin-1-yl)-1-oxoisoindolin-2-yl)piperidine-2,6-dione (1.5 mg, 2 μmol, 2.9% yield, 100% purity) was obtained as a white solid. LC-MS (ESI⁺) m/z: 732.3 (M+H)⁺.

LCMS: calc. for C₄₄H₅₃N₅O₅:731.94, found: [M+H]⁺ 732.3.

HPLC: 100% purity at 220 nm.

¹H NMR (400 MHZ, DMSO-d₆) δ=10.95 (s, 1H), 9.16 (br s, 1H), 8.25 (s, 0.204H), 7.53 (d, J=8.7 Hz, 1H), 7.08 (s, 2H), 6.97-6.90 (m, 2H), 6.89-6.83 (m, 2H), 6.55 (d, J=1.5 Hz, 1H), 6.49 (s, 2H), 5.05 (dd, J=5.1, 13.4 Hz, 1H), 4.80 (br d, J=6.2 Hz, 1H), 4.39-4.30 (m, 1H), 4.26-4.17 (m, 1H), 3.79 (s, 1H), 3.68 (br d, J=10.8 Hz, 2H), 3.33 (br s, 8H), 2.97-2.84 (m, 1H), 2.72-2.59 (m, 3H), 2.37 (br dd, J=4.4, 13.1 Hz, 1H), 2.24 (br d, J=7.0 Hz, 2H), 2.02-1.92 (m, 1H), 1.84 (br d, J=11.3 Hz, 2H), 1.71 (br s, 3H), 1.55 (br s, 2H), 1.43 (br d, J=6.4 Hz, 6H), 1.31-1.15 (m, 3H), 1.04-0.83 (m, 2H).

### Example 81. Preparation of (I-347) (S)-3-(5-(4-((1-(4-((3S,4R)-3-(2-fluorophenyl)-7-hydroxyisochroman-4-yl)phenyl)piperidin-4-yl)methyl)piperazin-1-yl)-1-oxoisoindolin-2-yl)piperidine-2,6-dione

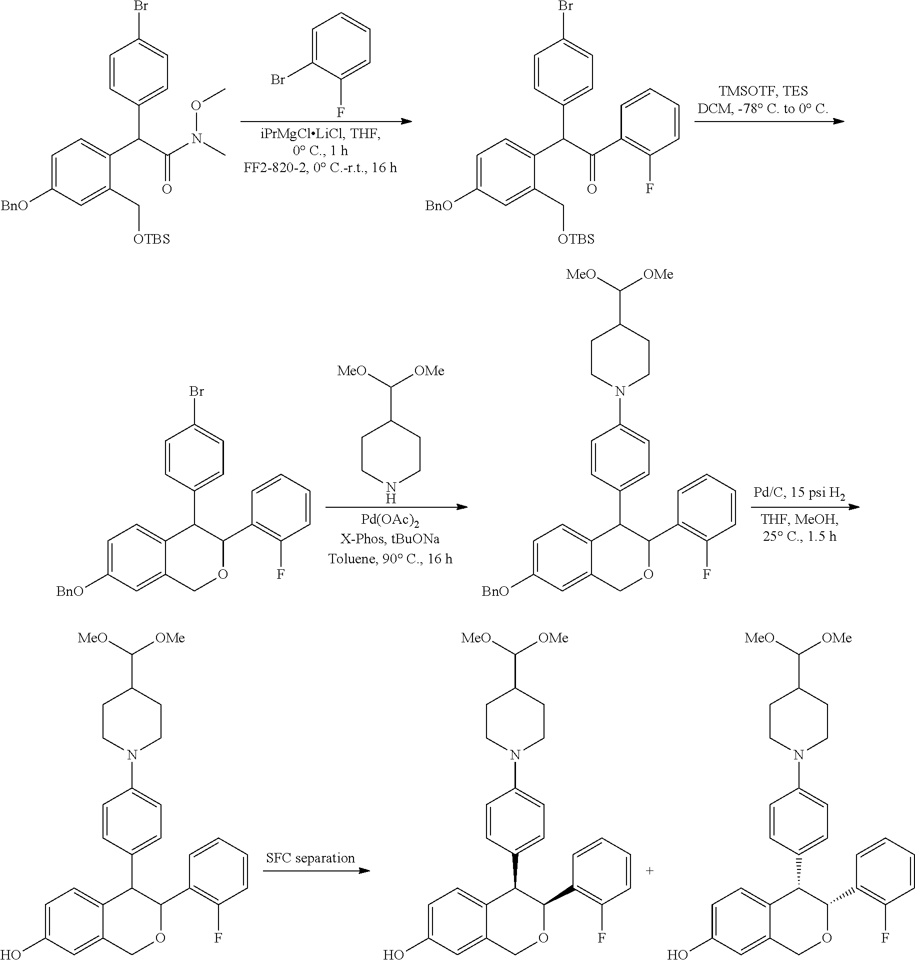

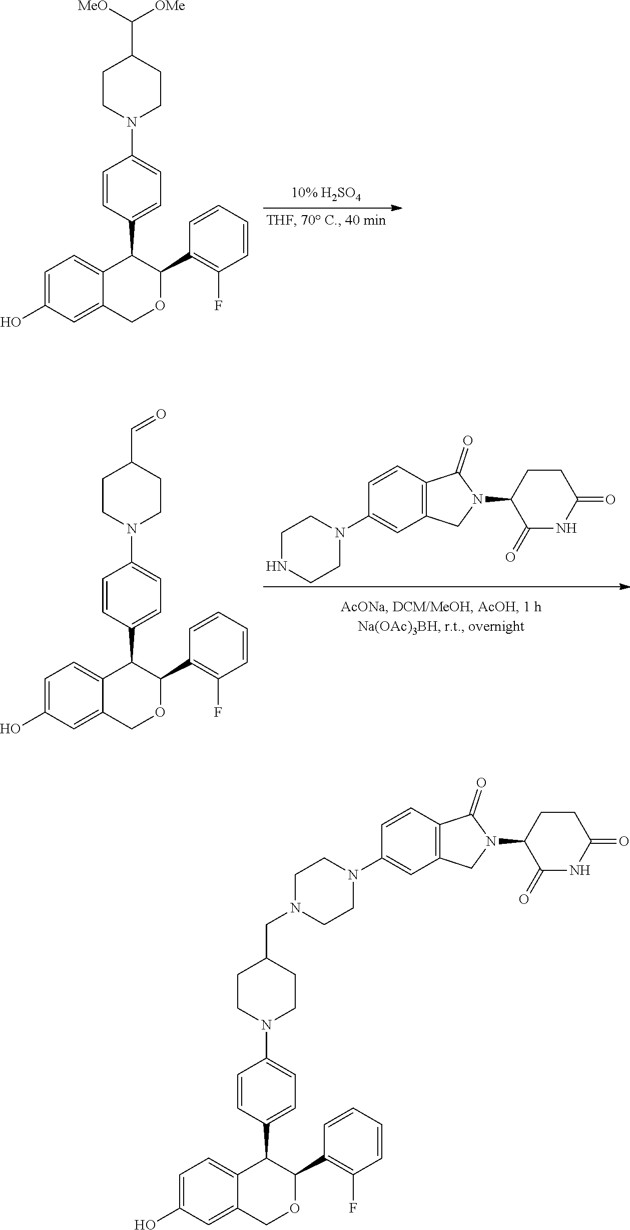 [SMILES: COC(OC)C1CCN(c2ccc([C@@H]3c4ccc(O)cc4CO[C@@H]3c3ccccc3F)cc2)CC1 ; O=CC1CCN(c2ccc([C@@H]3c4ccc(O)cc4CO[C@@H]3c3ccccc3F)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccccc7F)cc6)CC5)CC4)ccc3C2=O)C(=O)N1]

Step 1: A 8 mL thread vial was equipped with magnetic stirrer. (2-fluorophenyl) magnesium bromide was prepared as following procedure: iPrMgCl·LiCl (1.7 g, 8.8 mL, 1 Eq, 11 mmol, 1.3 M solution in THF) was cooled in an ice bath under N₂ atmosphere and 1-bromo-2-fluoro-benzene (2.0 g, 1.2 mL, 1 Eq, 11 mmol) was added dropwise. The reaction mixture was stirred at 0° C. for 1 hour. TLC (petroleum ether:ethyl acetate=1:0, UV) showed one new spot was observed.

A 40 mL thread vial was equipped with magnetic stirrer. 2-(4-(benzyloxy)-2-(((tert-butyldimethylsilyl)oxy)methyl)phenyl)-2-(4-bromophenyl)-N-methoxy-N-methylacetamide (1.0 g, 1 Eq, 1.7 mmol) was added in THF (4 mL) under N₂ atmosphere, then added (2-fluorophenyl) magnesium bromide (1.5 g, 4.5 Eq, 7.7 mmol) (prepared freshly) in the mixture at 0° C. for 10 min. The mixture was stirred at 25° C. for 16 hours. TLC (petroleum ether:ethyl acetate=5:1, UV) showed one main new spot was observed. The reaction solution was quenched with aq. NH₄Cl (15 mL). Water (100 mL) was added, and the mixture was extracted with dichloromethane (50 mL×3). The combined organic layers were washed with brine (50 mL), dried over anhydrous Na₂SO₄, filtered, and concentrated to dryness in vacuo to give a yellow oil. The yellow oil was purified by flash column (petroleum ether/ethyl acetate from 100/0 to 90/10) and the organic layer were concentrated in vacuo to give 2-(4-(benzyloxy)-2-(((tert-butyldimethylsilyl)oxy)methyl)phenyl)-2-(4-bromophenyl)-1-(2-fluorophenyl) ethan-1-one (314 mg, 499 μmol, 29% yield, 98% purity) as a yellow oil. LC-MS (ESI⁺) m/z: 487.1 (M+H)⁺.

Step 2: To a solution of 2-(4-(benzyloxy)-2-(((tert-butyldimethylsilyl)oxy)methyl)phenyl)-2-(4-bromophenyl)-1-(2-fluorophenyl) ethan-1-one (314 mg, 1 Eq, 499 μmol) and triethylsilane (87.1 mg, 120 μL, 1.5 Eq, 749 μmol) in DCM (4 mL), the resulting mixture was cooled at −78° C. and trimethylsilyl triflate (167 mg, 138 μL, 1.5 Eq, 749 μmol) was added, and the resulting mixture was allowed to stir for 30 min at this temperature. The reaction mixture was quenched with saturated aq. NaHCO₃ (5 mL), diluted with DCM (10 mL) and the resulting mixture stirred vigorously for 5 min. The organic layer was separated through a phase separator and concentrated in vacuo to give 7-(benzyloxy)-4-(4-bromophenyl)-3-(2-fluorophenyl)isochromane (330 mg, 517 μmol, crude product, 76% purity) as a yellow oil. LC-MS (ESI⁺) m/z: 488.8 (M+H)⁺.

Step 3: A mixture of 7-(benzyloxy)-4-(4-bromophenyl)-3-(2-fluorophenyl)isochromane (300 mg. 76% purity, 1 Eq, 470 μmol), 2-(Dicyclohexylphosphanyl)-2′,4′,6-tris(isopropyl) biphenyl (44.8 mg, 0.2 Eq, 94.0 μmol), palladium diacetate (15.8 mg, 0.15 Eq, 70.5 μmol), 4-(dimethoxymethyl)piperidine (112 mg, 118 μL, 1.5 Eq, 705 μmol), and sodium 2-methylpropan-2-olate (67.8 mg, 76.0 μL, 1.5 Eq, 705 μmol) in toluene (8 mL) was heated to 90° C. for 16 hours under N₂ atmosphere. TLC (petroleum ether:ethyl acetate=3:1, UV) showed one main new spot was observed. 100 mL water was added, and the mixture was extracted with DCM (150 mL×2). The combined organic layers were washed with brine (10 mL), dried over anhydrous Na₂SO₄, filtered and concentrated to dryness in vacuo to give a yellow oil. The yellow oil was purified by flash column (petroleum ether/ethyl acetate from 100/0 to 30/70) and the fraction was concentrated in vacuo to give 1-(4-(7-(benzyloxy)-3-(2-fluorophenyl)isochroman-4-yl)phenyl)-4-(dimethoxymethyl)piperidine (180 mg, 287 μmol, 61.2% yield, 90% purity) as a yellow oil. LC-MS (ESI+) m/z: 568.1 (M+H)⁺

Step 4: A mixture of 1-(4-(7-(benzyloxy)-3-(2-fluorophenyl)isochroman-4-yl)phenyl)-4-(dimethoxymethyl)piperidine (180 mg, 90% purity, 1 Eq, 287 μmol), Pd/C (180 mg, 10% Wt) in THF (2.5 mL) and MeOH (2.5 mL) was degassed and purged with H₂ for 3 times, and then the mixture was stirred at 25° C. for 2.5 hour under H₂ atmosphere (15 psi). The reaction was filtered and concentrated to dryness in vacuo to give 4-(4-(4-(dimethoxymethyl)piperidin-1-yl)phenyl)-3-(2-fluorophenyl)isochroman-7-ol (120 mg, 183 μmol, 63.7% yield, 73% purity) as a white solid.

LC-MS (ESI+) m/z: 478.3 (M+H)⁺

Step 5: The white solid was purified by SFC. Column: DAICEL CHIRALPAK IG (250 mm*30 mm, 10 um); Condition: CO₂-EtOH (0.1% NH₃H₂O); at the beginning: B (50%); at the end: B (50%); Flow rate (ml/min) 80. The aqueous phase was lyophilized to dryness to give (3S,4R)-4-(4-(4-(dimethoxymethyl)piperidin-1-yl)phenyl)-3-(2-fluorophenyl)isochroman-7-ol (40 mg, 70 μmol, 38% yield, 83% purity) as a colorless oil and (3R,4S)-4-(4-(4-(dimethoxymethyl)piperidin-1-yl)phenyl)-3-(2-fluorophenyl)isochroman-7-ol (40 mg, 81 μmol, 44% yield, 96% purity) as a colorless oil. LC-MS (ESI+) m/z: 478.1 (M+H)⁺.

Step 6: To a solution of (3*S,4*R)-4-(4-(4-(dimethoxymethyl)piperidin-1-yl)phenyl)-3-(2-fluorophenyl)isochroman-7-ol (40 mg, 83% purity, 1 Eq, 70 μmol) in THF (5 mL) was added sulfuric acid (69 mg, 5 mL, 10% Wt) at 25° C. Then the mixture was stirred at 70° C. for 40 min. The mixture was adjusted to pH 8-9 by progressively adding saturated aq. NaHCO₃ (10 mL). The reaction mixture was diluted with 50 mL H₂O and extracted with ethyl acetate (25 mL*2). The combined organic layers were washed with 10 mL (10 mL*1), dried over Na₂SO₄. The organic layers were filtered and concentrated under reduced pressure to give 1-(4-((3*S,4*R)-3-(2-fluorophenyl)-7-hydroxyisochroman-4-yl)phenyl)piperidine-4-carbaldehyde (40 mg, 72 μmol, crude product, 77% purity) as a colorless oil. LC-MS (ESI+) m/z: 450.3 (M+18)⁺

Step 7: A mixture of 1-(4-((3S,4R)-3-(2-fluorophenyl)-7-hydroxyisochroman-4-yl)phenyl)piperidine-4-carbaldehyde (40 mg, 77% purity, 1 Eq, 72 μmol), (S)-3-(1-oxo-5-(piperazin-1-yl)isoindolin-2-yl)piperidine-2,6-dione, benzene sulfonate (36 mg, 1 Eq, 72 μmol) and sodium acetate (29 mg, 5 Eq, 0.36 mmol) in DCM (2 mL) and MeOH (2 mL) at 20° C. for 0.5 hour, pH=8, then acetic acid (13 mg, 12 μL, 3 Eq, 0.22 mmol) at 20° C. for 1 hour, pH=6, then added sodium triacetoxyborohydride (30 mg, 2 Eq, 0.14 mmol) to the mixture, and then the mixture was stirred at 25° C. for 16 hour. The reaction was diluted with water (50 mL) and extracted with ethyl acetate (50 mL*2). The organic layers were concentrated to give crude product as a brown oil. The brown oil was purified by preparative high-performance liquid chromatography. Condition: water (FA)-ACN. Column: xtimate C18 150*40 mm*10 um. Begin B: 12%, end B: 42%. Gradient time (min) 2; 100% B hold Time (min) 2, Flow rate (ml/min) 25. The pure fractions were collected, and the solvent was evaporated under vacuum. The aqueous layer was lyophilized to dryness to give (S)-3-(5-(4-((1-(4-((3S,4R)-3-(2-fluorophenyl)-7-hydroxyisochroman-4-yl)phenyl)piperidin-4-yl)methyl)piperazin-1-yl)-1-oxoisoindolin-2-yl)piperidine-2,6-dione (17.2 mg, 22.7 μmol, 32% yield, 98% purity) as a white solid.

LCMS: calc. for C₄₄H₄₆FN₅O₅: 743.35, found: [M+H]⁺ 744.3.

HPLC: 98.23% purity at 220 nm.

¹H NMR (400 MHZ, DMSO-d₆) δ=10.94 (s, 1H), 9.32 (s, 1H), 8.13 (s, 0.377H), 7.51 (d, J=8.5 Hz, 1H), 7.20-7.08 (m, 2H), 7.07-7.03 (m, 2H), 6.90-6.77 (m, 3H), 6.63-6.53 (m, 6H), 5.19 (d, J=2.7 Hz, 1H), 5.12-5.01 (m, 2H), 4.94 (d, J=15.3 Hz, 1H), 4.37-4.28 (m, 1H), 4.23-4.15 (m, 1H), 4.01 (brs, 1H), 3.50 (br d, J=11.2 Hz, 2H), 3.30-3.22 (m, 5H), 2.95-2.84 (m, 1H), 2.63-2.51 (m, 3H), 2.49-2.30 (m, 4H), 2.26-2.15 (m, 2H), 2.00-1.91 (m, 1H), 1.74 (br d, J=12.4 Hz, 2H), 1.64 (br d, J=1.9 Hz, 1H), 1.21-1.08 (m, 2H).

### Example 82. Preparation of (I-348) (S)-3-(5-(4-((1-(4-((3R,4S)-3-(2-fluorophenyl)-7-hydroxyisochroman-4-yl)phenyl)piperidin-4-yl)methyl)piperazin-1-yl)-1-oxoisoindolin-2-yl)piperidine-2,6-dione

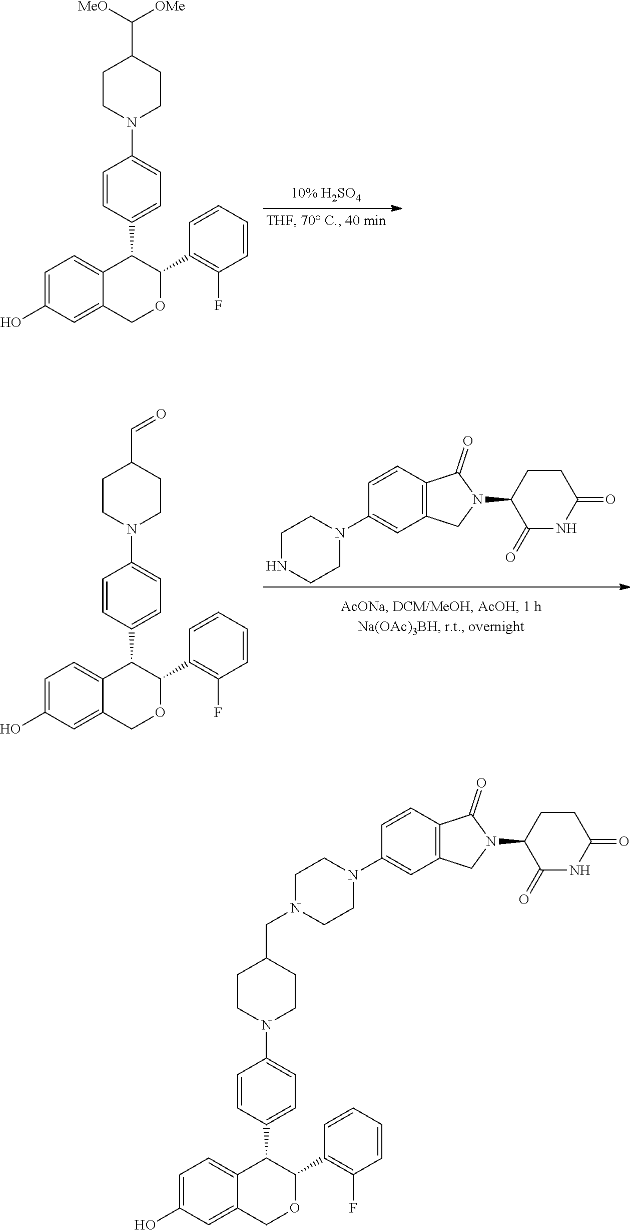 [SMILES: COC(OC)C1CCN(c2ccc([C@H]3c4ccc(O)cc4CO[C@H]3c3ccccc3F)cc2)CC1 ; O=CC1CCN(c2ccc([C@H]3c4ccc(O)cc4CO[C@H]3c3ccccc3F)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7c7ccccc7F)cc6)CC5)CC4)ccc3C2=O)C(=O)N1]

Step 1: To a solution of (3R,4S)-4-(4-(4-(dimethoxymethyl)piperidin-1-yl)phenyl)-3-(2-fluorophenyl)isochroman-7-ol (40 mg, 96% purity, 1 Eq, 81 μmol) n THF (5 mL) was added sulfuric acid (79 mg, 5 mL, 10% Wt) at 0° C. The mixture was stirred at 70° C. for 40 min. The mixture was adjusted to pH 8-9 by progressively adding saturated aq. NaHCO₃ (10 mL). The reaction mixture was diluted with 50 mL H₂O and extracted with ethyl acetate (50 mL). The combined organic layers were washed with 10 mL brine, dried over Na₂SO₄. The organic layers were filtered and concentrated under reduced pressure to give 1-(4-((3*R,4*S)-3-(2-fluorophenyl)-7-hydroxyisochroman-4-yl)phenyl)piperidine-4-carbaldehyde (33 mg, 74 μmol, 91% yield, crude product, 97% purity) as a colorless oil. LC-MS (ESI+) m/z: 450.3 (M+18)⁺.

Step 2: A mixture of 1-(4-((3R,4S)-3-(2-fluorophenyl)-7-hydroxyisochroman-4-yl)phenyl)piperidine-4-carbaldehyde (33 mg, 97% purity, 1 Eq, 74 μmol), (S)-3-(1-oxo-5-(piperazin-1-yl)isoindolin-2-yl)piperidine-2,6-dione, benzene sulfonate (37 mg, 1 Eq, 74 μmol) and sodium acetate (30 mg, 5 Eq, 0.37 mmol) in DCM (2 mL) and MeOH (2 mL) at 20° C. for 0.5 hour, pH=8, then acetic acid (13 mg, 13 μL, 3 Eq, 0.22 mmol) at 20° C. for 1 hour, pH=6, then added sodium triacetoxyborohydride (31 mg, 2 Eq, 0.15 mmol) the mixture, and then the mixture was stirred at 25° C. for 16 hour. The reaction was diluted with water (50 mL) and extracted with ethyl acetate (50 mL*2). The organic layers were concentrated to give crude product as a brown oil. The brown oil was purified by preparative high-performance liquid chromatography. Condition: water (FA)-ACN. Column: xtimate C18 150*40 mm*10 um. Begin B: 12%, end B: 42%. Gradient time (min) 2; 100% B hold time (min) 2, Flow rate (ml/min) 25. The pure fractions were collected, and the solvent was evaporated under vacuum. The aqueous layer was lyophilized to dryness to give (S)-3-(5-(4-((1-(4-((3R,4S)-3-(2-fluorophenyl)-7-hydroxyisochroman-4-yl)phenyl)piperidin-4-yl)methyl)piperazin-1-yl)-1-oxoisoindolin-2-yl)piperidine-2,6-dione (16.2 mg, 21.8 μmol, 29% yield, 100% purity) as a white solid.

LCMS: calc. for C₄₄H₄₆FN₅O₅: 743.35, found: [M+H]⁺ 744.3.

HPLC: 100 purity at 220 nm.

¹H NMR (400 MHZ, DMSO-d₆) δ=10.94 (s, 1H), 9.32 (br s, 1H), 8.15 (s, 0.554H), 7.51 (d, J=8.6 Hz, 1H), 7.21-7.08 (m, 2H), 7.07-7.02 (m, 2H), 6.79 (br d, J=8.1 Hz, 3H), 6.62-6.54 (m, 6H), 5.19 (d, J=3.1 Hz, 1H), 5.12-5.01 (m, 2H), 4.94 (d, J=15.1 Hz, 1H), 4.36-4.28 (m, 1H), 4.24-4.16 (m, 1H), 4.01 (br s, 1H), 3.53-3.47 (m, 2H), 3.27 (br s, 5H), 2.95-2.83 (m, 1H), 2.62-2.54 (m, 1H), 2.41 (br d, J=4.4 Hz, 5H), 2.39-2.30 (m, 1H), 2.18 (br d, J=6.8 Hz, 2H), 1.99-1.91 (m, 1H), 1.74 (br d, J=12.3 Hz, 2H), 1.68-1.56 (m, 1H), 1.13 (br d, J=11.6 Hz, 2H).

### Example 83. Preparation of (I-350) (S)-3-(5-(4-((1-(4-((3R,4S)-7-hydroxy-3-(4-(trifluoromethyl)phenyl)isochroman-4-yl)phenyl)piperidin-4-yl)methyl)piperazin-1-yl)-1-oxoisoindolin-2-yl)piperidine-2,6-dione

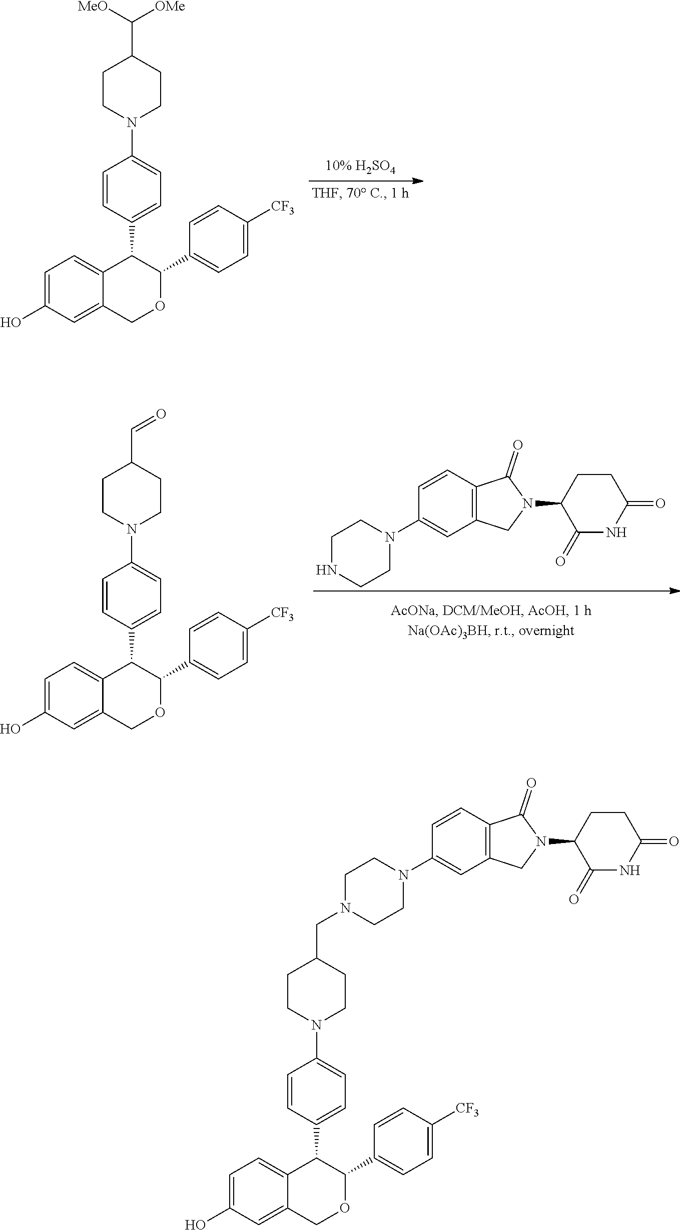 [SMILES: COC(OC)C1CCN(c2ccc([C@H]3c4ccc(O)cc4CO[C@H]3c3ccc(C(F)(F)F)cc3)cc2)CC1 ; O=CC1CCN(c2ccc([C@H]3c4ccc(O)cc4CO[C@H]3c3ccc(C(F)(F)F)cc3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7c7ccc(C(F)(F)F)cc7)cc6)CC5)CC4)ccc3C2=O)C(=O)N1]

Step 1: To a solution of (3R, 4S)-4-(4-(4-(dimethoxymethyl)piperidin-1-yl)phenyl)-3-(4-(trifluoromethyl)phenyl)isochroman-7-ol (120.0 mg, 1 Eq, 227.5 μmol) in THF (1.5 mL) and 10% H₂SO₄ (1.5 mL). The mixture was stirred at 70° C. for 1 hour. LCMS showed 99% of desired MS was detected. The aqueous phase was adjusted to pH˜=8 with NaHCO₃ (aq), and it was discarded. The reaction mixture was quenched by addition 50 mL H₂O at 25° C., and then diluted with 50 mL H₂O and extracted with ethyl acetate 100 mL (50 mL*2). The combined organic layers were washed with 10 mL brine and dried over Na₂SO₄, than was filtered and concentrated under reduced pressure to give 1-(4-((3R,4S)-7-hydroxy-3-(4-(trifluoromethyl)phenyl)isochroman-4-yl)phenyl)piperidine-4-carbaldehyde (110 mg, 228 μmol) as a residue. LC-MS (ESI+) m/z: 482.1 (M+H)⁺.

Step 2: To a solution of 1-(4-((3R,4S)-7-hydroxy-3-(4-(trifluoromethyl)phenyl)isochroman-4-yl)phenyl)piperidine-4-carbaldehyde (110.0 mg, 1 Eq, 228.4 μmol), (S)-3-(1-oxo-5-(piperazin-1-yl)isoindolin-2-yl)piperidine-2,6-dione benzenesulfonate (133.4 mg, 1.2 Eq, 274.1 μmol) in DCM (2 mL) and MeOH (2 mL) was added sodium acetate (56.22 mg, 3 Eq, 685.3 μmol) was stirred at 25° C. for 1 hour, Then sodium triacetoxyhydroborate (96.83 mg, 2 Eq, 456.9 μmol) and acetic acid (41.15 mg, 39.2 μL, 3 Eq, 685.3 μmol) was added. The mixture was stirred at 25° C. for 16 hour. LCMS showed 85% desired MS was detected. 20 mL water was added and the mixture was extracted with Ethyl acetate (30 mL×2). The combined organic layers were washed with brine (10 mL), dried over anhydrous Na₂SO₄, filtered and concentrated to dryness in vacuo to give a yellow oil. The yellow oil was concentrated under reduced pressure to remove solvent. The residue was purified by prep-HPLC (column: Welch Xtimate C18 150*30 mm*5 um phase: [water (FA)-ACN]; B %: 10%-50%, 9 min to give (S)-3-(5-(4-((1-(4-((3R,4S)-7-hydroxy-3-(4-(trifluoromethyl)phenyl)isochroman-4-yl)phenyl)piperidin-4-yl)methyl)piperazin-1-yl)-1-oxoisoindolin-2-yl)piperidine-2,6-dione (121.2 mg, 150 μmol, 65.8%, 98.4% Purity) as a white solid.

LCMS: calc. for C₄₅H₄₆F₃N₅O₅: 793.89, found: [M+H]⁺ 794.4.

HPLC: 98.4% purity at 220 nm.

¹H NMR 1H NMR (400 MHz, DMSO-d₆) δ=10.94 (s, 1H) 9.31 (br s, 1H) 8.15 (s, 0.420H) 7.52 (br d, J=8.03 Hz, 3H) 7.32 (br d, J=8.03 Hz, 2H) 6.99-7.09 (m, 2H) 6.80 (br d, J=8.03 Hz, 1H) 6.54-6.66 (m, 6H) 5.00-5.29 (m, 3H) 4.94 (br d, J=15.31 Hz, 1H) 4.26-4.40 (m, 1H) 4.14-4.25 (m, 1H) 4.11 (br s, 1H) 3.50 (br d, J=11.29 Hz, 2H) 3.28 (br s, 8H) 2.82-3.00 (m, 1H) 2.59 (br d, J=17.32 Hz, 1H) 2.45-2.49 (m, 2H) 2.31-2.40 (m, 1H) 2.19 (br d, J=6.53 Hz, 2H) 1.89-2.01 (m, 1H) 1.60-1.78 (m, 3H) 1.06-1.21 (m, 2H).

### Example 84. Preparation of (I-351) (S)-3-(5-(4-((1-(4-((3S,4R)-7-hydroxy-3-(2-isopropylphenyl)isochroman-4-yl)phenyl)piperidin-4-yl)methyl)piperazin-1-yl)-1-oxoisoindolin-2-yl)piperidine-2,6-dione and (I-352) (S)-3-(5-(4-((1-(4-((3R,4S)-7-hydroxy-3-(2-isopropylphenyl)isochroman-4-yl)phenyl)piperidin-4-yl)methyl)piperazin-1-yl)-1-oxoisoindolin-2-yl)piperidine-2,6-dione

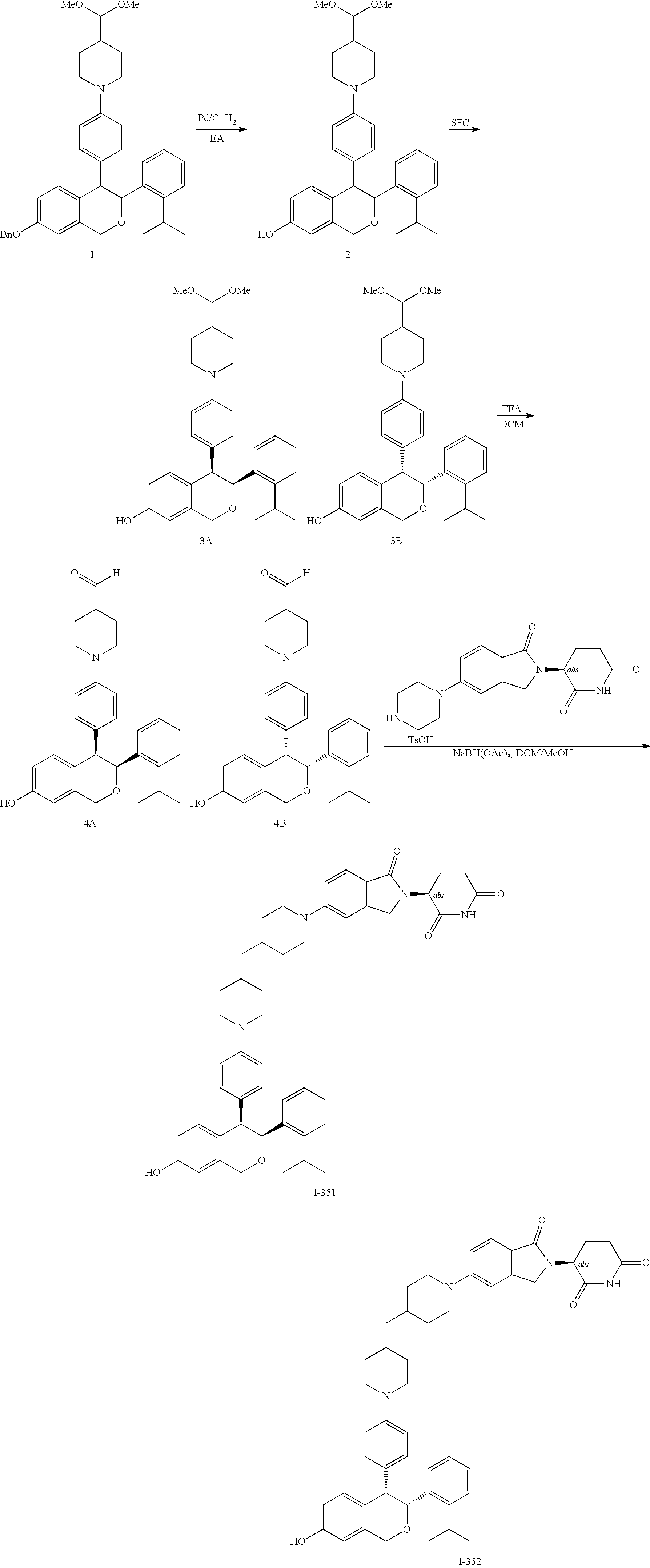

Step 1: A solution of 1(60 mg, 101.39 μmol, 1.0 eq.) and Pd/C (107.90 mg, 101.39 μmol, 10% purity, 1.0 eq.) in EA (3 mL) and then degassed and purged with H₂ for 3 times, and then the mixture was stirred at 20° C. for 3 hr under H₂ atmosphere. LCMS showed that the desired product was detected. The reaction mixture was filtered and concentrated under reduced pressure to get the product. The mixture was purified by Pre-TLC (PE/EA=1/1) to give 2 (45 mg, 80.76% yield) as colorless oil.

LC-MS (ESI+) m/z: 502.3 (M+H)⁺.

Step 2: The 2 (55 mg, 109.64 μmol, 1.0 eq.) was further separated by SFC (condition: CO₂-EtOH (0.1% NH₃H₂O), column: DAICEL CHIRALPAK AD (250 mm×30 mm, 10 um), Begin B: 30, End B 30) to give 3A (20 mg, 36.36% yield) as white solid and 3B (18 mg, 32.73% yield) as white solid.

3A: LC-MS (ESI⁺) m/z: 502.3 (M+H)⁺.

3B: LC-MS (ESI+) m/z: 502.3 (M+H)⁺.

Step 3: To a solution of 3A (18 mg, 35.88 μmol, 1.0 eq.) in DCM (2 mL) was added TFA (767.50 mg, 6.73 mmol, 187.59 eq.) at 20° C. Then the mixture was stirred at 20° C. for 1 h. LCMS showed that the starting material was consumed, and the desired product was detected. The mixture was filtered and concentrated under vacuum to give 4A (15 mg, crude) as yellow oil.

LC-MS (ESI+) m/z: 456.2 (M+H)⁺

Step 4: To a solution of 3B (15 mg, 29.90 μmol, 1.0 eq.) in DCM (2 mL) was added TFA (767.50 mg, 6.73 mmol, 225.11 eq.) at 20° C. Then the mixture was stirred at 20° C. for 1 h. LCMS showed that the starting material was consumed, and the desired product was detected. The mixture was filtered and concentrated under vacuum to give 4B (12 mg, crude) as yellow oil.

LC-MS (ESI+) m/z: 456.2 (M+H)⁺

Step 5: To a solution of 1-(4-((3S,4R)-7-hydroxy-3-(2-isopropylphenyl)isochroman-4-yl)phenyl)piperidine-4-carbaldehyde (15 mg, 32.92 μmol, 1.0 eq.) in DCE (5 mL) and MeOH (5 mL) was added (S)-3-(1-oxo-5-(piperazin-1-yl)isoindolin-2-yl)piperidine-2,6-dione, TsOH salt (21.62 mg, 65.85 μmol, 2.0 eq.) at 20° C. After 5 minutes, the sodium;triacetoxyboranuide (34.89 mg, 164.62 μmol, 5.0 eq.) was added into the mixture at 20° C. The mixture was stirred at 20° C. for 0.5 h. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 12, End B 42, Gradient Time (min) 12, 100% B Hold Time (min) 2, Flow Rate (mL/min) 25.) to give (S)-3-(5-(4-((1-(4-((3S,4R)-7-hydroxy-3-(2-isopropylphenyl)isochroman-4-yl)phenyl)piperidin-4-yl)methyl)piperazin-1-yl)-1-oxoisoindolin-2-yl)piperidine-2,6-dione (11.3 mg, 44.69% yield) as white solid. LC-MS (ESI+) m/z: 768.4 (M+H)⁺

LCMS: calc. for C₄₇H₅₃N₅O₅:767.97, found: [M+H]⁺ 768.4.

HPLC: 100.00% purity at 220 nm

¹H NMR (400 MHZ, DMSO-d₆) δ=10.94 (s, 1H), 9.29 (s, 1H), 8.13 (s, 1H), 7.61-7.28 (m, 2H), 7.23 (d, J=7.2 Hz, 1H), 7.07 (brt, J=7.0 Hz, 2H), 6.81-6.72 (m, 2H), 6.62-6.48 (m, 7H), 5.17 (d, J=2.4 Hz, 1H), 5.10-5.01 (m, 2H), 4.97-4.90 (m, 1H), 4.37-4.17 (m, 2H), 3.90 (d, J=1.9 Hz, 1H), 3.56-3.45 (m, 2H), 3.29 (s, 10H), 3.19-2.85 (m, 2H), 2.43-2.31 (m, 4H), 2.25-2.13 (m, 2H), 1.99-1.93 (m, 1H), 1.74 (br d, J=12.4 Hz, 3H), 1.34 (d, J=6.7 Hz, 3H), 1.15 (br d, J=6.7 Hz, 3H).

Step 6: To a solution of 1-(4-((3R,4S)-7-hydroxy-3-(2-isopropylphenyl)isochroman-4-yl)phenyl)piperidine-4-carbaldehyde (12 mg, 26.34 μmol, 1.0 eq.) in DCE (5 mL) and MeOH (5 mL) was added (S)-3-(1-oxo-5-(piperazin-1-yl)isoindolin-2-yl)piperidine-2,6-dione, TsOH salt (17.30 mg, 52.68 μmol, 2.0 eq.) at 20° C. After 5 minutes, the sodium;triacetoxyboranuide (27.91 mg, 131.70 μmol, 5.0 eq.) was added into the mixture at 20° C. The mixture was stirred at 20° C. for 0.5 h. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 12, End B 42, Gradient Time (min) 12, 100% B Hold Time (min) 2, Flow Rate (mL/min) 25.) to give (S)-3-(5-(4-((1-(4-((3R,4S)-7-hydroxy-3-(2-isopropylphenyl)isochroman-4-yl)phenyl)piperidin-4-yl)methyl)piperazin-1-yl)-1-oxoisoindolin-2-yl)piperidine-2,6-dione (11.7 mg, 56.75% yield) as white solid. LC-MS (ESI+) m/z: 768.4 (M+H)⁺

LCMS: calc. for C₄₇H₅₃N₅O₅:767.97, found: [M+H]⁺ 768.4.

HPLC: 98.11% purity at 220 nm

¹H NMR (400 MHZ, DMSO-d₆) δ=10.94 (s, 1H), 9.29 (s, 1H), 8.13 (s, 1H), 7.60-7.30 (m, 2H), 7.23 (d, J=7.7 Hz, 1H), 7.07 (brt, J=7.0 Hz, 2H), 6.80-6.73 (m, 2H), 6.62-6.49 (m, 7H), 5.17 (d, J=2.3 Hz, 1H), 5.09-5.01 (m, 2H), 4.97-4.90 (m, 1H), 4.36-4.18 (m, 2H), 3.90 (br d, J=0.7 Hz, 1H), 3.51 (br d, J=9.4 Hz, 2H), 3.29 (br s, 10H), 3.19-3.13 (m, 1H), 2.95-2.85 (m, 1H), 2.45-2.31 (m, 4H), 2.26-2.12 (m, 2H), 1.99-1.93 (m, 1H), 1.79-1.62 (m, 3H), 1.34 (d, J=6.8 Hz, 3H), 1.15 (br d, J=6.7 Hz, 3H).

### Example 85. Preparation of (I-393) 3-[6-[4-[[1-[4-[(3S,4R)-7-hydroxy-3-phenyl-isochroman-4-yl]phenyl]-4-piperidyl]methyl]piperazin-1-yl]-1-methyl-indazol-3-yl]piperidine-2,6-dione

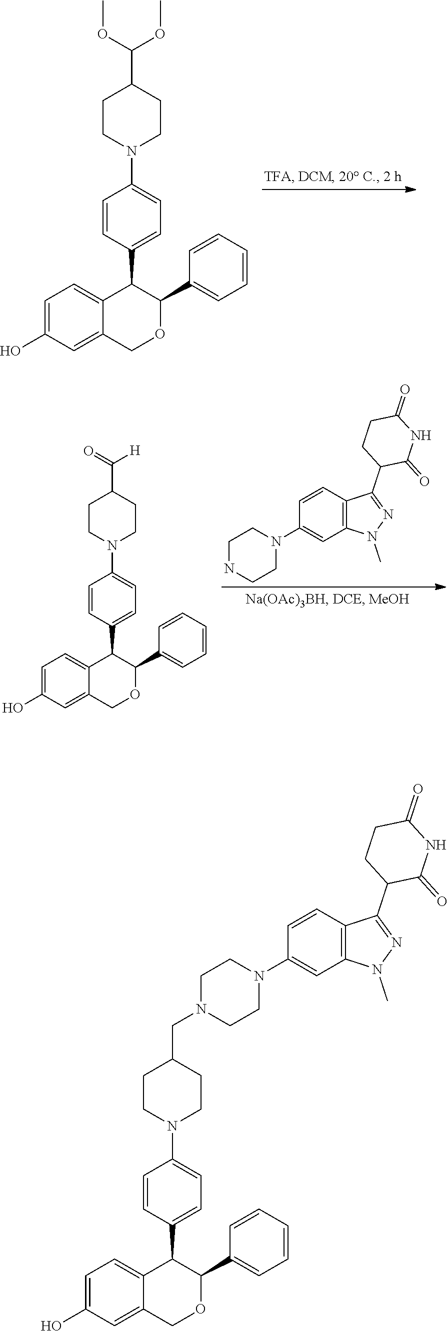 [SMILES: COC(OC)C1CCN(c2ccc([C@@H]3c4ccc(O)cc4CO[C@@H]3c3ccccc3)cc2)CC1 ; O=CC1CCN(c2ccc([C@@H]3c4ccc(O)cc4CO[C@@H]3c3ccccc3)cc2)CC1 ; Cn1nc(C2CCC(=O)NC2=O)c2ccc(N3CCNCC3)cc21 ; Cn1nc(C2CCC(=O)NC2=O)c2ccc(N3CCN(CC4CCN(c5ccc([C@@H]6c7ccc(O)cc7CO[C@@H]6c6ccccc6)cc5)CC4)CC3)cc21]

Step 1: To a solution of (3S,4R)-4-[4-[4-(dimethoxymethyl)-1-piperidyl]phenyl]-3-phenyl-isochroman-7-ol (80 mg, 1 eq, 174.07 μmol) in DCM (2 mL) was added TFA (921.00 mg, 46.4 eq. 8.08 mmol, 600.00 μL) and the mixture was stirred at 20° C. for 2 h. The reaction mixture was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give 1-[4-[(3S,4R)-7-hydroxy-3-phenyl-isochroman-4-yl]phenyl]piperidine-4-carbaldehyde (71 mg, crude) as a brown oil. LC-MS (ESI+) m/z: 414.2 (M+H)⁺.

Step 2: To a solution of 1-[4-[(3S,4R)-7-hydroxy-3-phenyl-isochroman-4-yl]phenyl]piperidine-4-carbaldehyde (70 mg, 1 eq, 169.28 μmol) in DCE (10 mL) and MeOH (1 mL) was added 3-(1-methyl-6-piperazin-1-yl-indazol-3-yl)piperidine-2,6-dione (55.42 mg, 1 eq, 169.28 μmol) and stirred at 20° C. for 0.5 h. Then NaBH(OAc)₃ (107.63 mg, 3 eq, 507.85 μmol) was added and stirred at 20° C. for 0.5 h. The solvent was removed to yield a residue which was purified by preparative HPLC (column: Phenomenex Gemini NX 150×30 mm, 5 um; mobile phase: [water(NH₄HCO₃)-ACN]; gradient: 50%-77% B over 11 min), followed by lyophilization to give the product of 3-[6-[4-[[1-[4-[(3S,4R)-7-hydroxy-3-phenyl-isochroman-4-yl]phenyl]-4-piperidyl]methyl]piperazin-1-yl]-1-methyl-indazol-3-yl]piperidine-2,6-dione (31 mg, 24.69% yield) as a white solid. LC-MS (ESI+) m/z: 725.3 (M+H)⁺.

LCMS: calc. for C₄₄H₄₈N₆O₄:724.91, found: [M+H]⁺ 725.3.

HPLC: 98.907% purity at 254 nm.

¹HNMR (400 MHz, DMSO-d₆): δ 10.85 (s, 1H), 9.28 (s, 1H), 7.49 (d, J=8.9 Hz, 1H), 7.17-7.11 (m, 2H), 7.11-7.04 (m, 3H), 6.91 (d, J=9.3 Hz, 1H), 6.83 (s, 1H), 6.78 (d, J=8.0 Hz, 1H), 6.64-6.59 (m, 2H), 6.55 (d, J=6.1 Hz, 4H), 5.07 (d, J=15.4 Hz, 1H), 5.00 (d, J=2.6 Hz, 1H), 4.90 (d, J=15.4 Hz, 1H), 4.25 (dd, J=5.1, 8.9 Hz, 1H), 4.03 (d, J=2.5 Hz, 1H), 3.88 (s, 3H), 3.50 (d, J=11.9 Hz, 2H), 3.32 (s, 6H), 3.20 (s, 4H), 2.65-2.56 (m, 2H), 2.31-2.10 (m, 4H), 1.75 (d, J=12.4 Hz, 2H), 1.63 (s, 1H), 1.15 (d, J=11.2 Hz, 2H).

### Example 86. Preparation of (I-271) (S)-3-(5-(4-((1-(4-((3R,4S)-7-hydroxy-3′,4′-dihydro-2′H-spiro[isochromane-3,1′-naphthalen]-4-yl)phenyl)piperidin-4-yl)methyl)piperazin-1-yl)-1-oxoisoindolin-2-yl)piperidine-2,6-dione

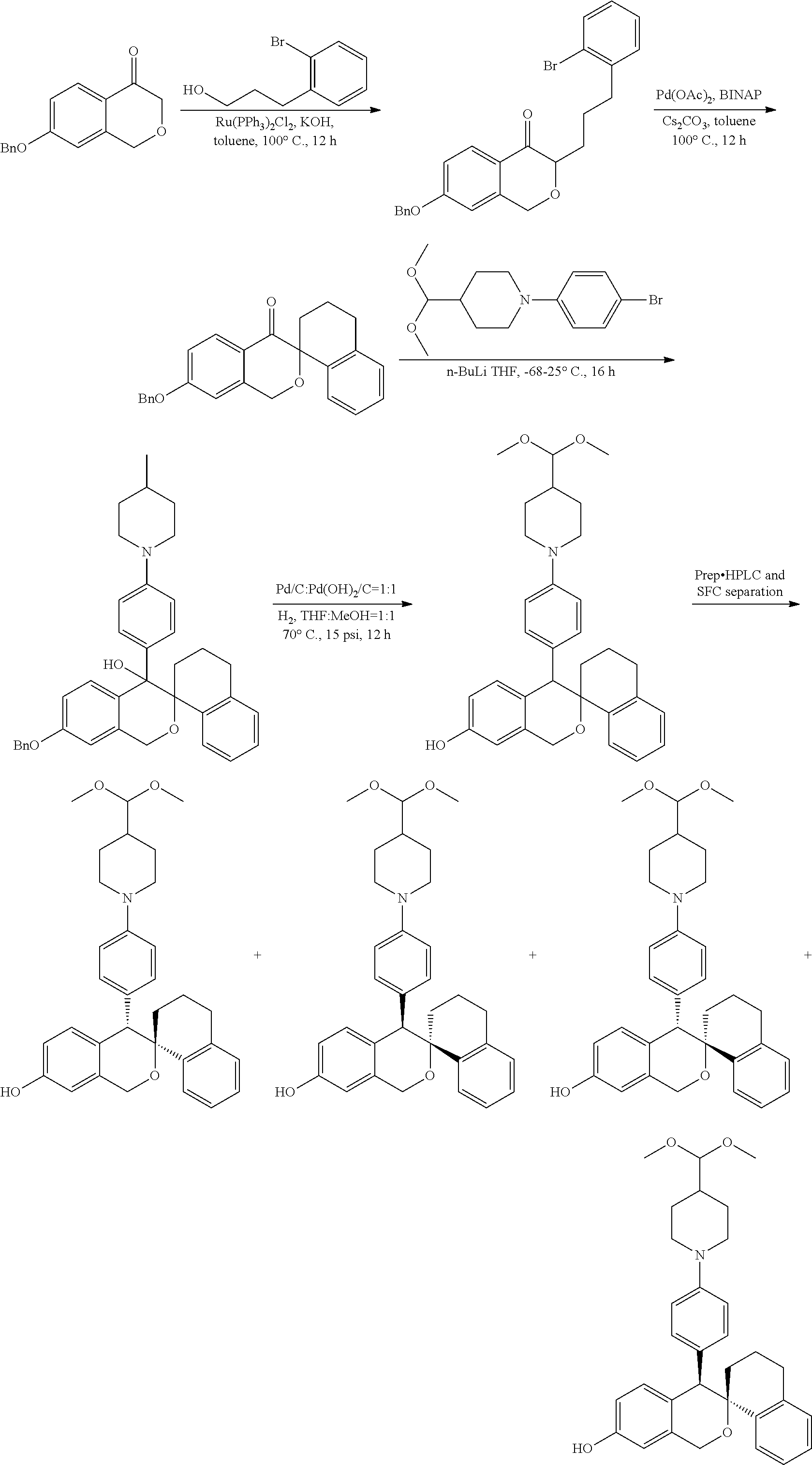

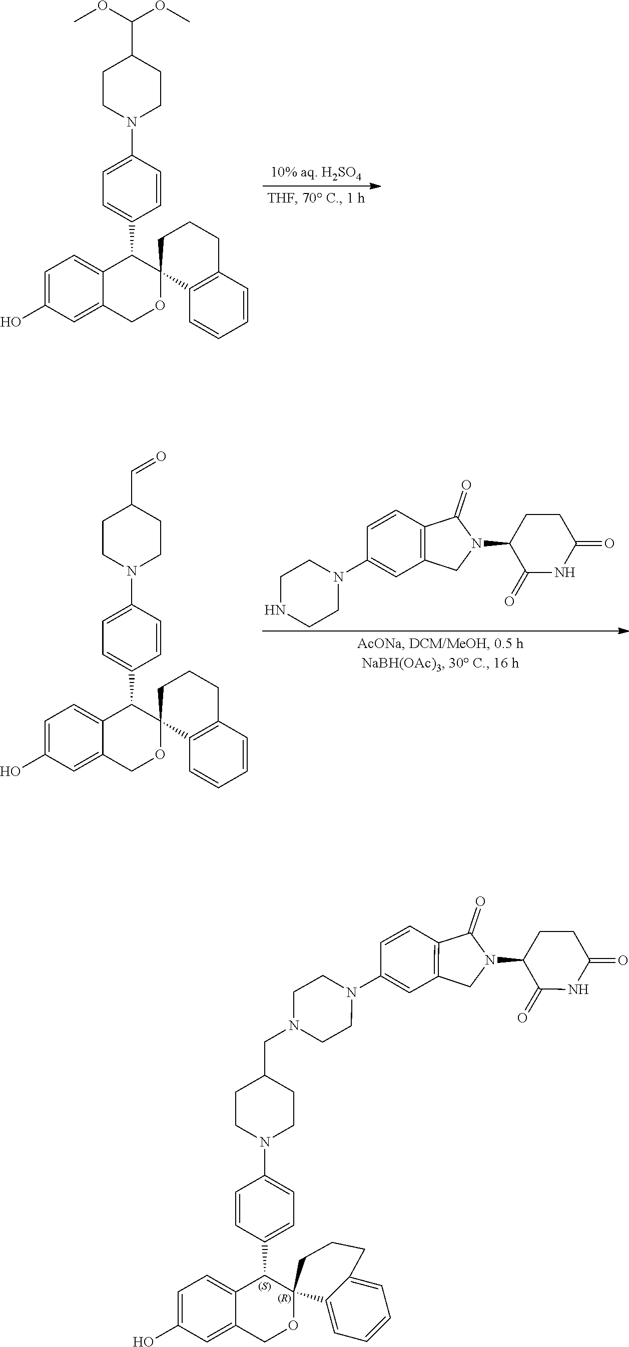

Step 1: A mixture of 7-(benzyloxy)isochroman-4-one (2.3 g, 9.1 mmol, 1 eq), 3-(2-bromophenyl) propan-1-ol (2.3 g, 10.8 mmol, 1.2 eq), KOH (508 mg, 9.1 mmol, 1 eq), Ru(PPh₃)₂Cl₂ (826 mg, 904.5 μmol, 0.1 eq) was added in toluene (25 mL) was purged N₂ 3 times, the mixture was stirred at 100° C. for 12 h. TLC (petroleum ether:ethyl acetate=10:1, R_{f}=0.7, UV) showed one main new spot was observed. The resulting solution was diluted with 50 mL H₂O and was extracted with ethyl acetate (100 mL*2), the organic layers were dried with anhydrous Na₂SO₄, filtered, and concentrated to give the crude product. The residue was subjected to column chromatography over silica gel (gradient elution: 0-20% EtOAc), The desired fractions were collected, and concentrated to dryness in vacuo to give 7-(benzyloxy)-3-(3-(2-bromophenyl) propyl)isochroman-4-one (3 g, 6.7 mmol, 73.5% yield) was obtained as a yellow oil.

Step 2: A mixture of 7-(benzyloxy)-3-(3-(2-bromophenyl) propyl)isochroman-4-one (2 g, 4.4 mmol, 1 eq), Cs₂CO₃ (2.9 g, 8.9 mmol, 2 eq), Pd(OAc)₂ (100 mg, 443.1 μmol, 0.1 eq), [1-(2-diphenylphosphanyl-1-naphthyl)-2-naphthyl]-diphenyl-phosphane (414 mg, 664.7 μmol, 0.15 eq) was added in toluene (100 mL) and was degassed and purged N₂ for 3 times, the mixture was stirred at 100° C. for 12 h. TLC (petroleum ether:ethyl acetate=10:1, R_{f}=0.7, UV) showed one main new spot was observed. The resulting solution was quenched with saturated aqueous NH₄Cl (20 mL) and was treated with 50 mL H₂O with extracted with 100 mL ethyl acetate. The organic layers were dried with anhydrous Na₂SO₄, filtered, and concentrated to give the crude product. The residue was purified by prep-HPLC (column: Welch Xtimate C18 150*30 mm*5 um; mobile phase: [water (FA)-ACN]; gradient: 60%-100% B over 9 min). Compound 7-(benzyloxy)-3′,4′-dihydro-2′H-spiro[isochromane-3, l′-naphthalen]-4-one (190 mg, 512.9 μmol, 11.6% yield) was obtained as a yellow oil.

Step 3: N-butyllithium (2.5 M, 615.49 μL, 3 eq) was added in the mixture of 1-(4-bromophenyl)-4-(dimethoxymethyl)piperidine (564 mg, 1.8 mmol, 3.5 eq) in THF (4 mL) at −78° C. under N₂, the mixture was stirred at −78° C. for 0.5 h, and then 7-(benzyloxy)-3′,4′-dihydro-2′H-spiro[isochromane-3,1′-naphthalen]-4-one (190 mg, 512.9 μmol, 1 eq) was added in the mixture at −78° C. under N₂, and was stirred at 25° C. for 12 h. TLC (petroleum ether:ethyl acetate=10:1, R_{f}=0.5 UV) showed one main new spot was observed. The resulting solution was quenched with saturated aqueous NH₄Cl (20 mL) adjust to pH=7 at 0° C., was diluted with H₂O (50 mL) extracted with ethyl acetate 100 mL, The organic layers were dried with anhydrous Na₂SO₄, filtered, and concentrated to give the crude product. The residue was subjected to column chromatography over silica gel (gradient elution: 0-20% EtOAc), The desired fractions were collected, and concentrated to dryness in vacuo to give 7-(benzyloxy)-4-(4-(4-(dimethoxymethyl)piperidin-1-yl)phenyl)-3′,4′-dihydro-2′H-spiro[isochromane-3,1′-naphthalen]-4-ol (280 mg, 462.2 μmol, 90% yield) was obtained as a colorless oil. LC-MS (ESI+) m/z: 606.4 (M+H)⁺

Step 4: A mixture of 7-(benzyloxy)-4-(4-(4-(dimethoxymethyl)piperidin-1-yl)phenyl)-3′,4′-dihydro-2′H-spiro[isochromane-3,1′-naphthalen]-4-ol (280 mg, 462.2 μmol, 1 eq), Pd/C (280 mg, 263.1 μmol, 10% wt), Pd(OH)₂/C (280 mg, 20% wt, 2 mmol) was added in THF (4 mL) and MeOH (4 mL) under N₂ atmosphere, The suspension was degassed and purged with H₂ for 3 times. The mixture was stirred under H₂ (15 psi) at 70° C. for 12 hr. The reaction mixture was filtered and concentrated under reduced pressure to give the crude product. The crude product was used into the next step without further purification. Compound 4-(4-(4-(dimethoxymethyl)piperidin-1-yl)phenyl)-3′,4′-dihydro-2′H-spiro[isochromane-3, l′-naphthalen]-7-ol (200 mg, 400.3 μmol, 86.6% yield) was obtained as a white solid. LC-MS (ESI+) m/z: 500.3 (M+H)⁺.

Step 5: The residue was purified by prep-HPLC (column: Welch Xtimate C18 150*30 mm*5 um; mobile phase: [water (FA)-ACN]; gradient: 48%-78% B over 7 min) to give two fractions. Fraction one (140 mg) was a red solid and fraction two (60 mg) was a red solid.

Fraction one was further separated by SFC (column: ChiralPak IH, 250*30 mm, 10 um; mobile phase: [CO₂-EtOH (0.1% NH₃H₂O)]; B %: 30%, isocratic elution mode) to give compound (3R,4S)-4-(4-(4-(dimethoxymethyl)piperidin-1-yl)phenyl)-3′,4′-dihydro-2′H-spiro[isochromane-3,1′-naphthalen]-7-ol (40 mg, 80.1 μmol, 14.8% yield) as a white solid and compound (3S,4R)-4-(4-(4-(dimethoxymethyl)piperidin-1-yl)phenyl)-3′,4′-dihydro-2′H-spiro[isochromane-3,1′-naphthalen]-7-ol (50 mg, 100.1 μmol, 18.5% yield) as a white solid.

Fraction two was further separated by SFC (column: REGIS(S,S)WHELK-O1 (250 mm*25 mm, 10 um); mobile phase: [CO₂-EtOH (0.1% NH₃H₂O)]; B %: 45%, isocratic elution mode) to give compound (3S,4S)-4-(4-(4-(dimethoxymethyl)piperidin-1-yl)phenyl)-3′,4′-dihydro-2′H-spiro[isochromane-3,1′-naphthalen]-7-ol (20 mg, 40 μmol, 7.4% yield) as a white solid and compound (3R,4R)-4-(4-(4-(dimethoxymethyl)piperidin-1-yl)phenyl)-3′,4′-dihydro-2′H-spiro[isochromane-3,1′-naphthalen]-7-ol (10 mg, 20 μmol, 3.7% yield) as a white solid. LC-MS (ESI+) m/z: 500.3 (M+H)⁺.

Step 6: To a solution of (3R,4S)-4-(4-(4-(dimethoxymethyl)piperidin-1-yl)phenyl)-3′,4′-dihydro-2′H-spiro[isochromane-3, l′-naphthalen]-7-ol (40 mg, 80.1 μmol, 1 eq) in THF (4 mL) was added 10% H₂SO₄ (4 mL). The mixture was stirred at 70° C. for 1 h. The reaction mixture was quenched by saturated NaHCO₃ aqueous solution addition (20 mL) at 25° C., and then diluted with 20 mL H₂O and extracted with ethyl acetate 40 mL. The combined organic layer was washed with 20 mL brine, dried over anhydrous Na₂SO₄, filtered and concentrated under reduced pressure to give a residue. The crude product 1-(4-((3R,4S)-7-hydroxy-3′,4′-dihydro-2′H-spiro[isochromane-3,1′-naphthalen]-4-yl)phenyl)piperidine-4-carbaldehyde (30 mg, 66.1 μmol, 82.6% yield) as a yellow oil was used into the next step without further purification. LC-MS (ESI+) m/z: 454.2 (M+H)⁺.

Step 7: To a solution of 1-(4-((3R,4S)-7-hydroxy-3′,4′-dihydro-2′H-spiro[isochromane-3,1′-naphthalen]-4-yl)phenyl)piperidine-4-carbaldehyde (30 mg, 66.1 μmol, 1 eq) and (3S)-3-(1-oxo-5-piperazin-1-yl-isoindolin-2-yl)piperidine-2,6-dione (32 mg, 66.1 μmol, 1 eq, PhSO₃H salt) in DCM (4 mL) and MeOH (4 mL) was added sodium acetate (16 mg, 198.4 μmol, 3 eq). The mixture was stirred at 30° C. for 0.5 h. Then the mixture was added NaBH(OAc)₃ (28 mg, 132.3 μmol, 2 eq) and acetic acid (12 mg, 198.4 μmol, 11 μL, 3 eq). The mixture was stirred at 30° C. for 16 h finally. The reaction mixture was diluted with 20 mL H₂O and extracted with 40 mL DCM. The combined organic layer was washed with 20 mL brine, dried over Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 15%-45% B over 7 min). (S)-3-(5-(4-((1-(4-((3R,4S)-7-hydroxy-3′,4′-dihydro-2′H-spiro[isochromane-3, l′-naphthalen]-4-yl)phenyl)piperidin-4-yl)methyl)piperazin-1-yl)-1-oxoisoindolin-2-yl)piperidine-2,6-dione (12 mg, 15.67 μmol, 23.69% yield) was obtained as a white solid.

LC-MS (ESI+) m/z: 766.2 (M+H)⁺.

HPLC: 98.23%, purity at 220 nm.

¹H NMR (400 MHz, Methanol-d₄) δ ppm 8.38-8.42 (m, 0.197H) 7.66 (d, J=8.28 Hz, 1H) 7.09-7.14 (m, 2H) 6.93-7.03 (m, 2H) 6.79 (d, J=9.29 Hz, 1H) 6.66-6.70 (m, 2H) 6.58 (s, 3H) 6.50-6.56 (m, 3H) 5.08-5.15 (m, 1H) 5.02-5.08 (m, 2H) 4.42 (d, J=5.77 Hz, 2H) 4.10 (s, 1H) 3.51-3.59 (m, 2H) 3.43 (br s, 4H) 2.85 (s, 3H) 2.81 (br dd, J=4.52, 2.51 Hz, 1H) 2.76 (br s, 4H) 2.68 (s, 3H) 2.45 (br d, J=7.03 Hz, 2H) 2.37 (dt, J=12.23, 3.54 Hz, 1H) 2.12-2.21 (m, 1H) 1.94-2.09 (m, 2H) 1.75-1.91 (m, 4H) 1.32-1.43 (m, 2H) 1.16-1.27 (m, 2H).

### Example 87. Preparation of (I-76) (S)-3-(5-(4-((1-(4-((3S,4R)-7-hydroxy-3′,4′-dihydro-2′H-spiro[isochromane-3,1′-naphthalen]-4-yl)phenyl)piperidin-4-yl)methyl)piperazin-1-yl)-1-oxoisoindolin-2-yl)piperidine-2,6-dione

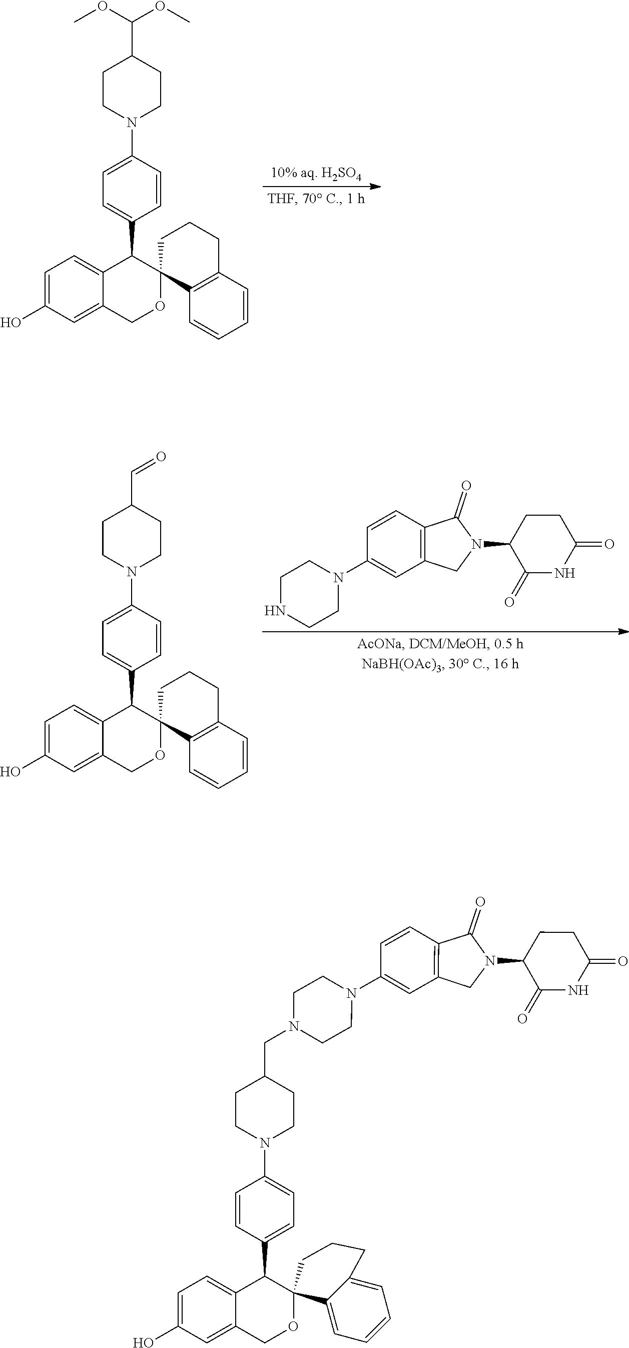 [SMILES: COC(OC)C1CCN(c2ccc([C@@H]3c4ccc(O)cc4CO[C@@]34CCCc3ccccc34)cc2)CC1 ; O=CC1CCN(c2ccc([C@@H]3c4ccc(O)cc4CO[C@@]34CCCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@]78CCCc7ccccc78)cc6)CC5)CC4)ccc3C2=O)C(=O)N1]

Step 1: To a solution of (3S,4R)-4-(4-(4-(dimethoxymethyl)piperidin-1-yl)phenyl)-3′,4′-dihydro-2′H-spiro[isochromane-3, l′-naphthalen]-7-ol (50 mg, 100.07 μmol, 1 eq) in THF (4 mL) was added 10% aq·H₂SO₄ (4 mL). The mixture was stirred at 70° C. for 1 h. The reaction mixture was quenched by saturated NaHCO₃ aqueous solution addition 20 mL at 25° C., and then diluted with 20 mL H₂O and extracted with 40 mL ethyl acetate. The combined organic layers were washed with 20 mL brine, dried over anhydrous Na₂SO₄, filtered and concentrated under reduced pressure to give a residue. The crude product 1-[4-[(3*R,4*S)-7-hydroxyspiro[isochromane-3,l′-tetralin]-4-yl]phenyl]piperidine-4-carbaldehyde (40 mg, 88.2 μmol, 88.1% yield) as a yellow oil was used into the next step without further purification.

LC-MS (ESI+) m/z: 472.2 (M+H₂O)⁺.

Step 2: To a solution of 1-(4-((3S,4R)-7-hydroxy-3′,4′-dihydro-2′H-spiro[isochromane-3,1′-naphthalen]-4-yl)phenyl)piperidine-4-carbaldehyde (40 mg, 88.2 μmol, 1 eq) and (3S)-3-(1-oxo-5-piperazin-1-yl-isoindolin-2-yl)piperidine-2,6-dione (43 mg, 88.2 μmol, 1 eq, PhSO₃H) in DCM (4 mL) and MeOH (4 mL) was added sodium acetate (22 mg, 264.5 μmol, 3 eq). The mixture was stirred at 30° C. for 0.5 h. Then the mixture was added NaBH(OAc)₃ (37 mg, 176.4 μmol, 2 eq) and acetic acid (16 mg, 264.6 μmol, 15 μL, 3 eq). The mixture was stirred at 30° C. for 16 h finally. The reaction mixture was diluted with 10 mL H₂O and extracted with 40 mL DCM. The combined organic layer was washed with 20 mL brine, dried over anhydrous Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 18%-48% B over 7 min). (S)-3-(5-(4-((1-(4-((3S,4R)-7-hydroxy-3′,4′-dihydro-2′H-spiro[isochromane-3, l′-naphthalen]-4-yl)phenyl)piperidin-4-yl)methyl)piperazin-1-yl)-1-oxoisoindolin-2-yl)piperidine-2,6-dione (5.3 mg, 6.9 μmol, 7.9% yield) was obtained as a white solid.

LC-MS (ESI+) m/z: 766.2 (M+H)⁺

HPLC: 98.738%, purity at 220 nm.

¹H NMR (400 MHZ, Methanol-d₄) δ ppm 8.33-8.38 (m, 0.277H) 7.64-7.69 (m, 1H) 7.08-7.14 (m, 2H) 6.99-7.03 (m, 1H) 6.93-6.97 (m, 1H) 6.77-6.81 (m, 1H) 6.66-6.70 (m, 2H) 6.57-6.64 (m, 3H) 6.49-6.56 (m, 3H) 5.08-5.15 (m, 1H) 5.03-5.07 (m, 2H) 4.37-4.48 (m, 2H) 4.10 (s, 1H) 3.56 (br d, J=11.54 Hz, 2H) 3.44 (br d, J=4.52 Hz, 4H) 2.86-2.99 (m, 3H) 2.82 (br d, J=4.77 Hz, 5H) 2.59-2.69 (m, 3H) 2.51 (br d, J=6.27 Hz, 2H) 2.37 (dt, J=12.42, 3.95 Hz, 1H) 2.13-2.21 (m, 1H) 1.95-2.09 (m, 2H) 1.80-1.92 (m, 4H) 1.34-1.44 (m, 2H).

### Example 88. Preparation of (I-272) (S)-3-(5-(4-((1-(4-((3S,4S)-7-hydroxy-3′,4′-dihydro-2′H-spiro[isochromane-3,1′-naphthalen]-4-yl)phenyl)piperidin-4-yl)methyl)piperazin-1-yl)-1-oxoisoindolin-2-yl)piperidine-2,6-dione

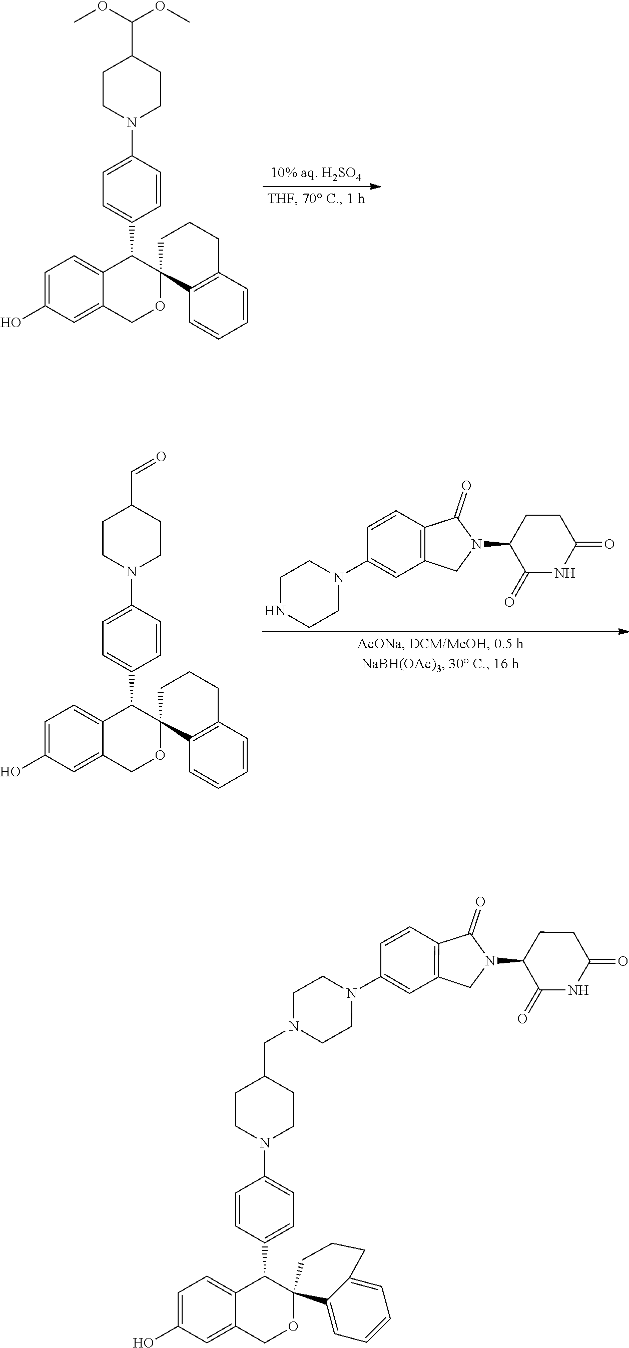 [SMILES: COC(OC)C1CCN(c2ccc([C@H]3c4ccc(O)cc4CO[C@@]34CCCc3ccccc34)cc2)CC1 ; O=CC1CCN(c2ccc([C@H]3c4ccc(O)cc4CO[C@@]34CCCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@@]78CCCc7ccccc78)cc6)CC5)CC4)ccc3C2=O)C(=O)N1]

Step 1: To a solution of (3S,4S)-4-(4-(4-(dimethoxymethyl)piperidin-1-yl)phenyl)-3′,4′-dihydro-2′H-spiro[isochromane-3,1′-naphthalen]-7-ol (20 mg, 40 μmol, 1 eq) in THF (4 mL) was added 10% aq·H₂SO₄ (4 mL). The mixture was stirred at 70° C. for 1 h. The reaction mixture was quenched by saturated NaHCO₃ aqueous solution addition 20 mL at 25° C., and then diluted with 20 mL H₂O and extracted with ethyl acetate. The combined organic layers were washed with 20 mL brine, dried over anhydrous Na₂SO₄, filtered and concentrated under reduced pressure to give a residue. The crude product 1-(4-((3S,4S)-7-hydroxy-3′,4′-dihydro-2′H-spiro[isochromane-3,1′-naphthalen]-4-yl)phenyl)piperidine-4-carbaldehyde (17 mg, 37.5 μmol, 93.6% yield) as a yellow oil was used into the next step without further purification.

LC-MS (ESI+) m/z: 472.1 (M+H₂O)⁺.

Step 2: To a solution of 1-(4-((3S,4S)-7-hydroxy-3′,4′-dihydro-2′H-spiro[isochromane-3,1′-naphthalen]-4-yl)phenyl)piperidine-4-carbaldehyde (17 mg, 37.5 μmol, 1 eq) and (3S)-3-(1-oxo-5-piperazin-1-yl-isoindolin-2-yl)piperidine-2,6-dione (18.2 mg, 37.5 μmol, 1 eq, PhSO₃H salt) in DCM (4 mL) and MeOH (4 mL) was added sodium acetate (9 mg, 112.4 μmol, 3 eq). The mixture was stirred at 30° C. for 0.5 h. Then the mixture was added NaBH(OAc)₃ (16 mg, 75 μmol, 2 eq) and acetic acid (7 mg, 112.4 μmol, 7 μL, 3 eq). The mixture was stirred at 30° C. for 16 h finally. The reaction mixture was diluted with 20 mL H₂O and extracted with 40 mL DCM. The combined organic layers were washed with 20 mL brine, dried over anhydrous Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 15%-45% B over 7 min). (S)-3-(5-(4-((1-(4-((3S,4S)-7-hydroxy-3′,4′-dihydro-2′H-spiro[isochromane-3, l′-naphthalen]-4-yl)phenyl)piperidin-4-yl)methyl)piperazin-1-yl)-1-oxoisoindolin-2-yl)piperidine-2,6-dione (1 mg, 1.3 μmol, 3.5% yield) was obtained as a white solid.

LC-MS (ESI+) m/z: 766.3 (M+H)⁺.

HPLC: 98.284%, purity at 220 nm.

¹H NMR (400 MHZ, Methanol-d₄) δ ppm 8.40-8.49 (m, 0.399H) 7.66 (d, J=8.28 Hz, 1H) 7.39 (d, J=7.28 Hz, 1H) 7.15-7.20 (m, 1H) 7.09-7.14 (m, 3H) 7.03-7.08 (m, 1H) 6.97 (d, J=8.78 Hz, 2H) 6.87 (br d, J=9.03 Hz, 3H) 6.60-6.65 (m, 1H) 6.44-6.48 (m, 1H) 5.08-5.15 (m, 1H) 4.76 (br s, 1H) 4.37-4.49 (m, 3H) 4.27 (s, 1H) 3.61-3.68 (m, 2H) 3.43 (br d, J=4.77 Hz. 4H) 2.79-2.96 (m, 3H) 2.65-2.77 (m, 7H) 2.48-2.60 (m, 2H) 2.45 (br d, J=6.53 Hz, 2H) 2.12-2.20 (m, 1H) 1.90-1.95 (m, 2H) 1.56-1.78 (m, 3H) 1.36-1.45 (m, 3H).

Absolute stereochemistry was arbitrarily assigned

### Example 89. Preparation of (I-273) (S)-3-(5-(4-((1-(4-((3R,4R)-7-hydroxy-3′,4′-dihydro-2′H-spiro[isochromane-3,1′-naphthalen]-4-yl)phenyl)piperidin-4-yl)methyl)piperazin-1-yl)-1-oxoisoindolin-2-yl)piperidine-2,6-dione

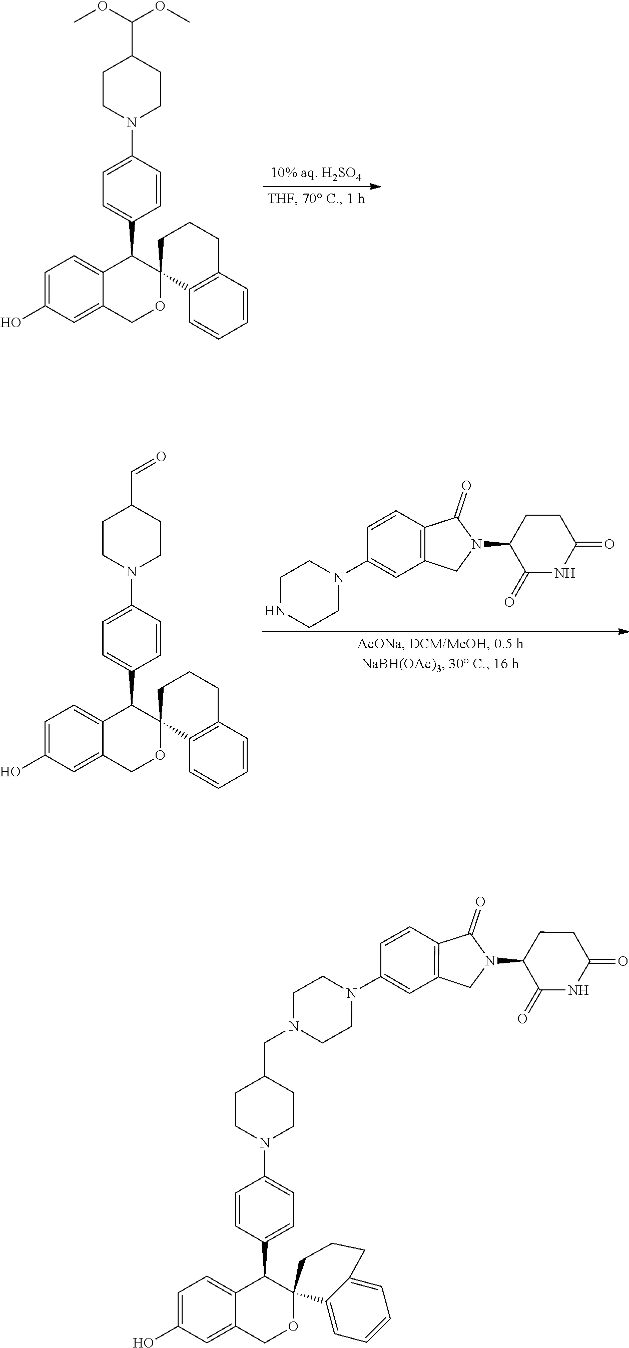 [SMILES: COC(OC)C1CCN(c2ccc([C@@H]3c4ccc(O)cc4CO[C@]34CCCc3ccccc34)cc2)CC1 ; O=CC1CCN(c2ccc([C@@H]3c4ccc(O)cc4CO[C@]34CCCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@]78CCCc7ccccc78)cc6)CC5)CC4)ccc3C2=O)C(=O)N1]

Step 1: To a solution of (3R,4R)-4-(4-(4-(dimethoxymethyl)piperidin-1-yl)phenyl)-3′,4′-dihydro-2′H-spiro[isochromane-3, l′-naphthalen]-7-ol (10 mg, 20 μmol, 1 eq) in THF (4 mL) was added 10% aq·H₂SO₄ (4 mL). The mixture was stirred at 70° C. for 1 h. The reaction mixture was quenched by saturated NaHCO₃ aqueous solution addition 20 mL at 25° C., and then diluted with 20 mL H₂O and extracted with 40 mL ethyl acetate. The combined organic layers were washed with 20 mL brine, dried over anhydrous Na₂SO₄, filtered and concentrated under reduced pressure to give a residue. The crude product 1-(4-((3R,4R)-7-hydroxy-3′,4′-dihydro-2′H-spiro[isochromane-3,1′-naphthalen]-4-yl)phenyl)piperidine-4-carbaldehyde (8 mg, 17.6 μmol, 88.1% yield) as a yellow oil was used into the next step without further purification.

LC-MS (ESI+) m/z: 472.1 (M+H₂O)⁺.

Step 2: To a solution of 1-(4-((3R,4R)-7-hydroxy-3′,4′-dihydro-2′H-spiro[isochromane-3,1′-naphthalen]-4-yl)phenyl)piperidine-4-carbaldehyde (8 mg, 17.6 μmol, 1 eq) and (3S)-3-(1-oxo-5-piperazin-1-yl-isoindolin-2-yl)piperidine-2,6-dione (8.6 mg, 17.6 μmol, 1 eq, PhSO₃H salt) in DCM (4 mL) and MeOH (4 mL) was added sodium acetate (4.3 mg, 52.9 μmol, 3 eq). The mixture was stirred at 30° C. for 0.5 h. Then the mixture was added NaBH(OAc); (8 mg, 35.3 μmol, 2 eq) and acetic acid (3 mg, 52.9 μmol, 3 μL, 3 eq). The mixture was stirred at 30° C. for 16 h finally. The reaction mixture was diluted with 20 mL H₂O and extracted with 40 mL DCM. The combined organic layer was washed with 20 mL brine, dried over anhydrous Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 15%-45% B over 7 min). (S)-3-(5-(4-((1-(4-((3R,4R)-7-hydroxy-3′,4′-dihydro-2′H-spiro[isochromane-3, l′-naphthalen]-4-yl)phenyl)piperidin-4-yl)methyl)piperazin-1-yl)-1-oxoisoindolin-2-yl)piperidine-2,6-dione (0.5 mg, 6.5e-1 μmol, 3.7% yield) was obtained as a white solid.

LC-MS (ESI+) m/z: 766.3 (M+H)⁺.

HPLC: 95.52%, purity at 220 nm.

¹H NMR (400 MHZ, Methanol-d₄) δ ppm 8.37-8.51 (m, 0.622H) 7.67 (d, J=8.28 Hz, 1H) 7.39 (d, J=7.78 Hz, 1H) 7.15-7.20 (m, 1H) 7.08-7.14 (m, 3H) 7.03-7.08 (m, 1H) 6.94-7.00 (m, 2H) 6.84-6.90 (m, 3H) 6.60-6.64 (m, 1H) 6.46 (d, J=2.51 Hz, 1H) 5.08-5.15 (m, 1H) 4.76 (s, 1H) 4.36-4.48 (m, 3H) 4.28 (d, J=15.31 Hz, 1H) 3.61-3.68 (m, 2H) 3.42 (br d, J=4.77 Hz, 4H) 2.79-2.95 (m, 3H) 2.71-2.79 (m, 5H) 2.65-2.70 (m, 2H) 2.48-2.64 (m, 2H) 2.43-2.47 (m, 2H) 2.13-2.21 (m, 1H) 1.89-1.96 (m, 2H) 1.57-1.79 (m, 3H) 1.37-1.45 (m, 3H). Absolute stereochemistry was arbitrarily assigned

### Example 90. Preparation of (I-353) (S)-3-(5-(4-((1-(4-((3R,4S)-7-hydroxy-3-(p-tolyl) isochroman-4-yl)phenyl)piperidin-4-yl)methyl)piperazin-1-yl)-1-oxoisoindolin-2-yl)piperidine-2,6-dione and (I-354) (S)-3-(5-(4-((1-(4-((3S,4R)-7-hydroxy-3-(p-tolyl) isochroman-4-yl)phenyl)piperidin-4-yl)methyl)piperazin-1-yl)-1-oxoisoindolin-2-yl)piperidine-2,6-dione

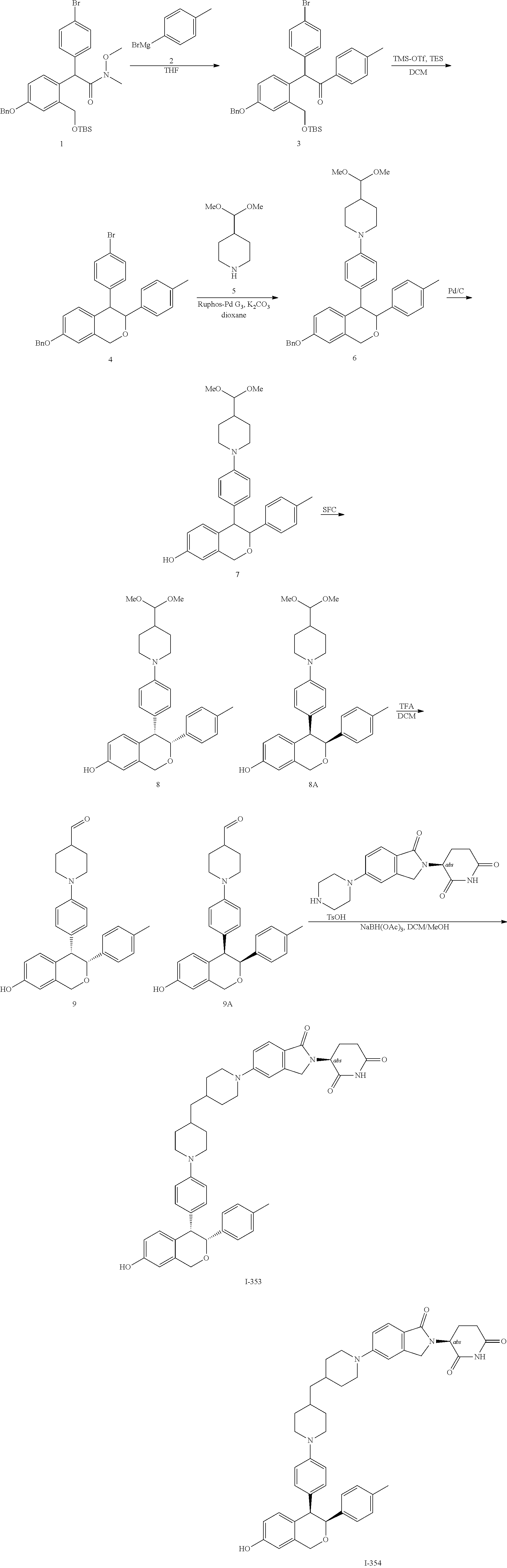

Step 1: To a solution of 1 (300 mg, 513.16 μmol, 1.0 eq.) in THF (4 mL) was added 2 (1 M, 1.54 mL, 3.0 eq.) at 0° C. under N₂. Then the mixture was stirred at 20° C. for 12 h under N₂. LCMS showed that the starting material was consumed, and the desired product was detected. MeOH (2 mL) was dropped into the mixture. Then the mixture was filtered and concentrated under vacuum to give the crude. The crude product was purified by Pre-TLC (PE/EA=10/1) to give 3 (220 mg, 55.07% yield) as colorless oil.

LC-MS (ESI+) m/z: 485.0 (M+H−130)⁺.

1H NMR (500 MHZ, DMSO-d₆) δ=7.87 (d, J=8.2 Hz, 2H), 7.51 (d, J=8.4 Hz, 2H), 7.42-7.24 (m, 7H), 7.13-7.04 (m, 3H), 6.88-6.78 (m, 2H), 6.34 (s, 1H), 5.05 (s, 2H), 4.81-4.52 (m, 2H), 2.33 (s, 3H), 0.84 (s, 9H), 0.01 (d, J=7.3 Hz, 6H).

Step 2: To a solution of 3 (220 mg, 357.33 μmol, 1.0 eq.) in DCM (10 mL) was added triethylsilane (124.65 mg, 1.07 mmol 3.0 eq.) at 20° C. The mixture was cooled to −78° C. Then the trimethylsilyl trifluoromethanesulfonate (119.13 mg, 536.00 μmol, 1.5 eq.) was added into the mixture at −78° C. The mixture was stirred at −78° C. for 1 h. TLC showed that no desired product was detected. Na₂CO₃ (sat aq.) (3 mL) was added into the mixture. The mixture was diluted and extracted with DCM mL (50 mL×3), dried over Na₂SO₄, filtered and concentrated under reduced pressure to give a residue. The mixture was purified by Pre-TLC (PE/EA=10/1) to give 4 (160 mg, 92.24% yield) as colorless oil.

¹H NMR (400 MHz, CHLOROFORM-d) 8=7.46-7.38 (m, 5H), 7.15 (d, J=8.3 Hz, 2H), 6.99-6.92 (m, 3H), 6.87-6.82 (m, 3H), 6.76 (d, J=2.3 Hz, 1H), 6.64 (d, J=8.5 Hz, 2H), 5.19-5.14 (m, 1H), 5.08-5.01 (m, 4H), 4.02 (d, J=3.1 Hz, 1H), 2.28 (s, 3H).

Step 3: To a solution of 4 (150 mg, 309.02 μmol, 1.0 eq.) in dioxane (1 mL) was added 5 (98.41 mg, 618.03 μmol, 2.0 eq.) and K₂CO₃ (128.12 mg, 927.05 μmol, 3.0 eq.) at 20° C. Then the [2-(2-aminophenyl)phenyl]-methylsulfonyloxy-palladium;dicyclohexyl-[2-(2,6-diisopropoxyphenyl)phenyl]phosphane (38.77 mg, 46.35 μmol, 0.15 eq.) was added into the mixture at 20° C. The mixture was stirred at 100° C. for 4 h under N₂. LCMS showed that the starting material was consumed and the desired product was detected. The mixture was filtered and concentrated under vacuum to give the crude. The crude was purified by Pre-TLC (PE/EA=3/1) to give 6 as yellow solid.

LC-MS (ESI+) m/z: 564.3 (M+H)⁺.

1H NMR (400 MHZ, chloroform-d) 8=7.48-7.33 (m, 7H), 6.95 (br d, J=7.7 Hz, 3H), 6.84 (br d, J=7.6 Hz, 3H), 6.75 (br s, 1H), 6.63 (br s, 2H), 5.19-5.14 (m, 1H), 5.07 (s, 2H), 5.01 (br d, J=16.6 Hz, 1H), 4.19-4.09 (m, 1H), 4.09-3.92 (m, 2H), 3.60-3.54 (m, 2H), 3.37 (s, 6H), 2.58-2.46 (m, 2H), 2.27 (s, 3H), 1.85-1.65 (m, 4H), 1.45-1.39 (m, 1H).

Step 4: A solution of 6 (120 mg, 212.87 μmol, 1.0 eq.) and Pd/C (226.54 mg, 212.87 μmol, 10% purity, 1.0 eq.) in EA (3 mL) and then degassed and purged with H₂ for 3 times, and then the mixture was stirred at 15° C. for 3 hr under H₂ atmosphere. LCMS showed that the starting material was consumed, and the desired product was detected. The mixture was filtered and concentrated under vacuum to give the crude. The crude was purified by Pre-TLC (PE/EA=1/1) to give 7 (50 mg, 47.99% yield) as colorless solid.

LC-MS (ESI+) m/z: 474.1 (M+H)⁺.

Step 5: The 7 (80 mg, 168.92 μmol, 1.0 eq.) was further separated by SFC (condition: CO₂-EtOH (0.1% NH₃H₂O), column: DAICEL CHIRALPAK AD (250 mm×30 mm, 10 um), Begin B: 25, End B: 25) to give (3R,4S)-4-(4-(4-(dimethoxymethyl)piperidin-1-yl)phenyl)-3-(p-tolyl) isochroman-7-ol 8 (30 mg, 37.50% yield) as white solid and (3S,4R)-4-(4-(4-(dimethoxymethyl)piperidin-1-yl)phenyl)-3-(p-tolyl)isochroman-7-ol 8A (30 mg, 37.50% yield) as white solid.

8: LC-MS (ESI+) m/z: 474.4 (M+H)⁺.

1H NMR (400 MHZ, DMSO-d₆) δ=10.94 (s, 1H), 9.29 (s, 1H), 8.13 (s, 1H), 7.60-7.30 (m, 2H), 7.23 (d, J=7.7 Hz, 1H), 7.07 (br t, J=7.0 Hz, 2H), 6.80-6.73 (m, 2H), 6.62-6.49 (m, 7H), 5.17 (d, J=2.3 Hz, 1H), 5.09-5.01 (m, 2H), 4.97-4.90 (m, 1H), 4.36-4.18 (m, 2H), 3.90 (br d, J=0.7 Hz, 1H), 3.51 (br d, J=9.4 Hz, 2H), 3.29 (br s, 10H), 3.19-3.13 (m, 1H), 2.95-2.85 (m, 1H), 2.45-2.31 (m, 4H), 2.26-2.12 (m, 2H), 1.99-1.93 (m, 1H), 1.79-1.62 (m, 3H), 1.34 (d, J=6.8 Hz, 3H), 1.15 (br d, J=6.7 Hz, 3H).

8A: LC-MS (ESI⁺) m/z: 474.4 (M+H)⁺.

1H NMR (400 MHZ, DMSO-d₆) δ=9.26 (s, 1H), 6.94 (s, 4H), 6.77 (d, J=8.1 Hz, 1H), 6.65-6.61 (m, 2H), 6.57-6.53 (m, 4H), 5.05 (d, J=15.5 Hz, 1H), 4.94 (d, J=3.1 Hz, 1H), 4.88 (d, J=15.4 Hz, 1H), 4.04 (d, J=6.6 Hz, 1H), 3.99 (d, J=2.7 Hz, 1H), 3.51 (br d, J=11.2 Hz, 2H), 3.29 (s, 2H), 3.24 (s, 6H), 2.19 (s, 3H), 1.67-1.60 (m, 3H), 1.23 (br d, J=9.1 Hz, 2H).

Step 6: To a solution of (3R,4S)-4-(4-(4-(dimethoxymethyl)piperidin-1-yl)phenyl)-3-(p-tolyl) isochroman-7-ol 8 (25 mg, 1.0 Eq, 53 μmol) in DCM (1 mL) was added Trifluoroacetic acid (0.4 g, 0.3 mL, 7e+1 Eq, 4 mmol) at 15° C. Then the mixture was stirred at 15° C. for 1 hour. LCMS showed that the desired product was detected. Then the mixture was filtered and concentrated under vacuum to give the 1-(4-((3R,4S)-7-hydroxy-3-(p-tolyl)isochroman-4-yl)phenyl)piperidine-4-carbaldehyde 9 (20 mg, crude).

LC-MS (ESI+) m/z: 446.2 (M+H+18)⁺

Step 7: To a solution of (3S,4R)-4-(4-(4-(dimethoxymethyl)piperidin-1-yl)phenyl)-3-(p-tolyl) isochroman-7-ol 8A (25 mg, 1.0 Eq, 53 μmol) in DCM (1 mL) was added Trifluoroacetic acid (0.4 g, 0.3 mL, 7e+1 Eq, 4 mmol) at 15° C. Then the mixture was stirred at 15° C. for 1 hour. LCMS showed that the desired product was detected. Then the mixture was filtered and concentrated under vacuum to give the 1-(4-((3S,4R)-7-hydroxy-3-(p-tolyl)isochroman-4-yl)phenyl)piperidine-4-carbaldehyde 9A (20 mg, crude).

LC-MS (ESI+) m/z: 446.2 (M+H+18)⁺

Step 8: To a solution of 1-(4-((3R,4S)-7-hydroxy-3-(p-tolyl)isochroman-4-yl)phenyl)piperidine-4-carbaldehyde 9 (20 mg, 1 Eq, 47 μmol) in DCE (1 mL) and MeOH (1 mL) was added 10 (31 mg, 2 eq, 94 μmol) and Sodium triacetoxyborohydride (50 mg, 5 Eq, 0.23 mmol) at 15° C. Then the mixture was stirred at 15° C. for 1 hour. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 12, 100% B Hold Time (min) 2, Flow Rate (mL/min) 25.) to give (S)-3-(5-(4-((1-(4-((3R,4S)-7-hydroxy-3-(p-tolyl)isochroman-4-yl)phenyl)piperidin-4-yl)methyl)piperazin-1-yl)-1-oxoisoindolin-2-yl)piperidine-2,6-dione (27.7 mg, 79% yield) as white solid. LC-MS (ESI⁺) m/z: 740.4 (M+H)⁺

LCMS: calc. for C₄₅H₄₉N₅O₅: 739.92, found: [M+H]⁺ 740.4.

HPLC: 98.84% purity at 220 nm

1H NMR (400 MHz, DMSO-d₆) δ=10.95 (s, 1H), 9.28 (s, 1H), 8.13 (s, 1H), 7.60-7.54 (m, 1H), 7.38-7.24 (m, 1H), 7.09 (br s, 1H), 6.95 (s, 4H), 6.77 (d, J=8.1 Hz, 1H), 6.67-6.61 (m, 2H), 6.59-6.52 (m, 4H), 5.09-5.00 (m, 2H), 4.97-4.86 (m, 2H), 4.36-4.18 (m, 2H), 4.00 (br d, J=2.1 Hz, 1H), 3.52 (br d, J=10.5 Hz, 2H), 3.32-3.07 (m, 8H), 2.94-2.86 (m, 1H), 2.60 (br d, J=2.1 Hz, 4H), 2.43-2.30 (m, 2H), 2.19 (s, 3H), 2.00-1.93 (m, 1H), 1.76 (br d, J=12.0 Hz, 3H), 1.26-1.09 (m, 2H).

Step 9: To a solution of 1-(4-((3S,4R)-7-hydroxy-3-(p-tolyl)isochroman-4-yl)phenyl)piperidine-4-carbaldehyde 9A (25 mg, 1 Eq, 58 μmol) in DCE (1 mL) and MeOH (1 mL) was added 10 (38 mg, 2 Eq, 0.12 mmol) and Sodium triacetoxyborohydride (62 mg, 5 eq, 0.29 mmol) at 15° C. Then the mixture was stirred at 15° C. for 1 hour. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 12, 100% B Hold Time (min) 2, Flow Rate (mL/min) 25.) to give (S)-3-(5-(4-((1-(4-((3S,4R)-7-hydroxy-3-(p-tolyl)isochroman-4-yl)phenyl)piperidin-4-yl)methyl)piperazin-1-yl)-1-oxoisoindolin-2-yl)piperidine-2,6-dione (28.2 mg, 65% yield) as white solid. LC-MS (ESI+) m/z: 740.4 (M+H)⁺

LCMS: calc. for C₄₅H₄₉N₅O₅:739.92, found: [M+H]⁺ 740.4.

HPLC: 99.38% purity at 220 nm

¹H NMR (400 MHZ, DMSO-d₆) δ=10.95 (s, 1H), 9.28 (s, 1H), 8.13 (s, 1H), 7.52 (br d, J=8.5 Hz, 2H), 7.07-7.05 (m, 1H), 6.95 (s, 4H), 6.77 (d, J=8.1 Hz, 1H), 6.66-6.62 (m, 2H), 6.58-6.52 (m, 4H), 5.08-5.02 (m, 2H), 4.96-4.86 (m, 2H), 4.35-4.18 (m, 2H), 4.00 (br d, J=2.0 Hz, 1H), 3.51 (br d, J=11.1 Hz, 2H), 3.33-3.20 (m, 8H), 2.94-2.85 (m, 1H), 2.60 (br s, 4H), 2.41-2.31 (m, 2H), 2.19 (s, 3H), 1.99-1.92 (m, 1H), 1.80-1.62 (m, 3H), 1.23-1.11 (m, 2H).

### Example 91. Preparation of (I-394) (R)-3-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)piperidine-2,6-dione and (I-395) (S)-3-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)piperidine-2,6-dione

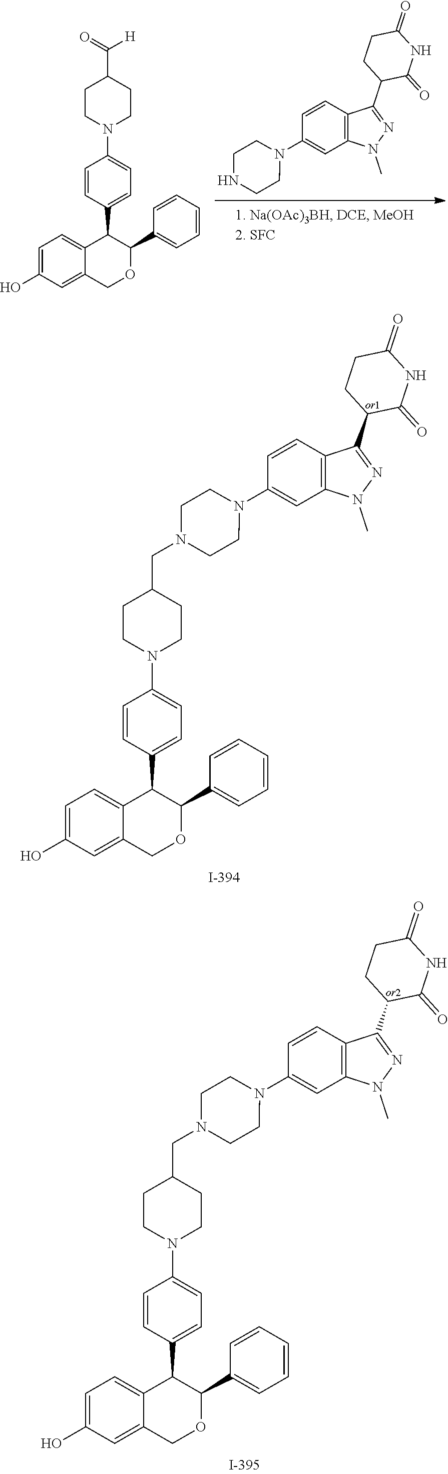

Step 1: To a solution of 1-[4-[(3S,4R)-7-hydroxy-3-phenyl-isochroman-4-yl]phenyl]piperidine-4-carbaldehyde (220 mg, 1 eq, 532.03 μmol) in DCM (10 mL) and MeOH (1 mL) was added 3-(1-methyl-6-piperazin-1-yl-indazol-3-yl)piperidine-2,6-dione (193.58 mg, 1 eq, 532.03 μmol, HCl) and stirred at 20° C. for 0.5 h. Then NaBH(OAc)₃ (338.28 mg, 3 eq, 1.60 mmol) was added and stirred at 20° C. for 0.5 h. The solvent was removed to yield a residue. The residue was purified by preparative HPLC (column: Phenomenex Gemini NX 150×30 mm, 5 um; mobile phase: [water(NH₄HCO₃)-ACN]; gradient: 50%-77% B over 11 min), followed by lyophilization to yield a product which was separated by chiral SFC (column: DAICEL CHIRALPAK ID (250 mm*30 mm, 10 um); mobile phase: [MeOH-ACN]B %: 50% %, isocratic elution mode, peak 1:2.324 min, peak 2:3.447 min) to yield Peak 1 and Peak 2.

Peak 1 was concentrated under reduced pressure to give the product of (R)-3-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)piperidine-2,6-dione or (S)-3-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)piperidine-2,6-dione (49.8 mg, 41.5% yield, SFC: Rt=2.324) as a white solid. LC-MS (ESI+) m/z: 725.3 (M+H)⁺.

LCMS: calc. for C₄₄H₄₈N₆O₄:724.91, found: [M+H]⁺ 725.3.

HPLC: 100.0% purity at 254 nm.

¹HNMR (500 MHz, DMSO-d₆): § 10.84 (s, 1H), 9.28 (s, 1H), 7.49 (d, J=9.0 Hz, 1H), 7.17-7.03 (m, 5H), 6.91 (dd, J=1.6, 9.1 Hz, 1H), 6.83 (s, 1H), 6.78 (d, J=8.2 Hz, 1H), 6.64-6.59 (m, 2H), 6.58-6.52 (m, 4H), 5.07 (d, J=15.4 Hz, 1H), 5.00 (d, J=3.1 Hz, 1H), 4.91 (d, J=15.3 Hz, 1H), 4.25 (dd, J=5.1, 9.2 Hz, 1H), 4.03 (d, J=2.7 Hz, 1H), 3.88 (s, 3H), 3.50 (d, J=11.9 Hz, 2H), 3.30 (s, 6H), 3.20 (s, 4H), 2.65-2.58 (m, 2H), 2.34-2.25 (m, 1H), 2.23-2.09 (m, 3H), 1.75 (d, J=11.6 Hz, 2H), 1.63 (s, 1H), 1.22-1.08 (m, 2H)

Peak 2 was concentrated under reduced pressure to give the product of (R)-3-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)piperidine-2,6-dione or (S)-3-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)piperidine-2,6-dione (40.9 mg, 33.7% yield, SFC: Rt=3.447) as a white solid. LC-MS (ESI+) m/z: 725.3 (M+H)⁺.

LCMS: calc. for C₄₄H₄₈N₆O₄:724.91, found: [M+H]⁺ 725.3.

HPLC: 100.0% purity at 254 nm.

¹HNMR (500 MHz, DMSO-d₆): δ 10.84 (s, 1H), 9.28 (s, 1H), 7.49 (d, J=9.0 Hz, 1H), 7.18-7.02 (m, 5H), 6.91 (dd, J=1.8, 9.0 Hz, 1H), 6.83 (d, J=1.4 Hz, 1H), 6.78 (d, J=8.2 Hz, 1H), 6.63-6.59 (m, 2H), 6.58-6.50 (m, 4H), 5.07 (d, J=15.6 Hz, 1H), 5.00 (d, J=3.1 Hz, 1H), 4.91 (d, J=15.3 Hz, 1H), 4.25 (dd, J=5.2, 9.2 Hz, 1H), 4.03 (d, J=2.9 Hz, 1H), 3.88 (s, 3H), 3.50 (d, J=11.9 Hz, 2H), 3.29 (s, 6H), 3.20 (s, 4H), 2.64-2.57 (m, 2H), 2.34-2.24 (m, 1H), 2.23-2.10 (m, 3H), 1.75 (d, J=11.4 Hz, 2H), 1.63 (d, J=3.5 Hz, 1H), 1.15 (q, J=12.1 Hz, 2H).

### Example 92. Preparation of (I-398) 3-(5-(4-((1-(4-((3S,4R)-7-hydroxy-3-phenylisochroman-4-yl)phenyl)piperidin-4-yl)methyl)piperazin-1-yl)-1H-indazol-1-yl)piperidine-2,6-dione

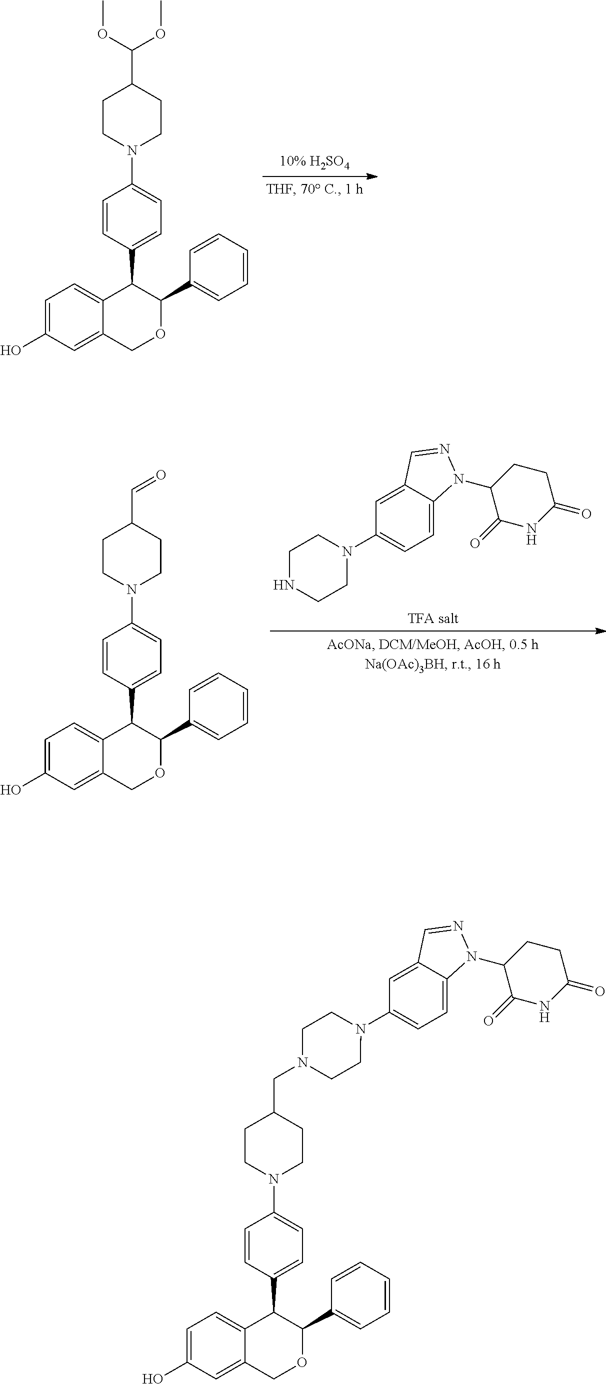 [SMILES: COC(OC)C1CCN(c2ccc([C@@H]3c4ccc(O)cc4CO[C@@H]3c3ccccc3)cc2)CC1 ; O=CC1CCN(c2ccc([C@@H]3c4ccc(O)cc4CO[C@@H]3c3ccccc3)cc2)CC1 ; O=C1CCC(n2ncc3cc(N4CCNCC4)ccc32)C(=O)N1 ; O=C1CCC(n2ncc3cc(N4CCN(CC5CCN(c6ccc([C@@H]7c8ccc(O)cc8CO[C@@H]7c7ccccc7)cc6)CC5)CC4)ccc32)C(=O)N1]

Step 1: To a solution of (3S,4R)-4-(4-(4-(dimethoxymethyl)piperidin-1-yl)phenyl)-3-phenylisochroman-7-ol (200 mg, 1 Eq, 435 μmol) in THE (5 mL) was added sulfuric acid (5 mL, 10% v/v) at 0° C. The mixture was stirred at 70° C. for 1 hour. The reaction mixture was adjusted to pH=˜8 by adding saturated aq. NaHCO₃ (10 mL). The mixture was diluted with 50 mL H₂O and extracted with 50 mL ethyl acetate. The organic layers were washed with brine 10 mL and dried over anhydrous Na₂SO₄. The organic layers were filtered and concentrated under reduced pressure to give 1-(4-((3S,4R)-7-hydroxy-3-phenylisochroman-4-yl)phenyl)piperidine-4-carbaldehyde (220 mg, 494 μmol, 93% purity, crude product) as a brown oil. LC-MS (ESI++) m/z: 432.1 (M+18)⁺.

Step 2: A mixture of 1-(4-((3S,4R)-7-hydroxy-3-phenylisochroman-4-yl)phenyl)piperidine-4-carbaldehyde (120 mg, 93% purity, 1 Eq, 269 μmol), 3-(5-(piperazin-1-yl)-1H-indazol-1-yl)piperidine-2,6-dione, trifluoroacetic acid salt (115 mg, 1 Eq, 269 μmol) and sodium acetate (110 mg, 5 Eq, 1.35 mmol) were added in DCM (2 mL) and MeOH (2 mL) at 25° C. for 0.5 hour, then added acetic acid (48.5 mg, 46.2 μL, 3 Eq, 808 μmol) and sodium triacetoxyborohydride (114 mg, 2 Eq, 538 μmol). The mixture was stirred at r.t. for 16 hours. The reaction was diluted with water (50 mL) and extracted with ethyl acetate (50 mL*2). The combined organic layers were concentrated to give crude product as a brown oil. The brown oil was purified by preparative high-performance liquid chromatography. Condition: water (FA)-ACN. Column: C18 150×30 mm. Begin B: 20%, End B: 50%. Gradient time (min) 6; 100% B hold time (min) 1, Flow rate (ml/min) 30. The aqueous layer was lyophilized to dryness to give 3-(5-(4-((1-(4-((3S,4R)-7-hydroxy-3-phenylisochroman-4-yl)phenyl)piperidin-4-yl)methyl)piperazin-1-yl)-1H-indazol-1-yl)piperidine-2,6-dione (78.2 mg, 110 μmol, 40.8% yield, 99.78% purity) as a white solid.

LCMS: calc. for C₄₃H₄₆N₆O₄:710.36, found: [M+H]⁺ 711.2.

HPLC: 99.78% purity at 220 nm.

¹H NMR (400 MHZ, DMSO-d₆) δ=11.12 (s, 1H), 9.27 (s, 1H), 8.19 (s, 1H), 8.13 (s, 0.364H), 7.47 (br d, J=9.3 Hz, 1H), 7.17-7.04 (m, 6H), 6.90 (br s, 1H), 6.78 (d, J=8.0 Hz, 1H), 6.64-6.59 (m, 2H), 6.55 (br d, J=6.8 Hz, 4H), 5.62 (br dd, J=5.1, 11.3 Hz, 1H), 5.07 (br d, J=15.7 Hz, 1H), 5.00 (d, J=2.5 Hz, 1H), 4.91 (br d, J=15.4 Hz, 1H), 4.03 (br d, J=1.8 Hz, 1H), 3.50 (br d, J=11.4 Hz, 2H), 3.08 (br d, J=1.3 Hz, 3H), 2.86-2.78 (m, 1H), 2.74-2.55 (m, 6H), 2.48-2.41 (m, 3H), 2.35-2.20 (m, 3H), 1.80-1.71 (m, 2H), 1.69-1.59 (m, 1H), 1.22-1.09 (m, 2H).

### Example 93. Preparation of (I-397) 3-(5-(4-((1-(4-((3S,4R)-7-hydroxy-3-phenylisochroman-4-yl)phenyl)piperidin-4-yl)methyl)piperazin-1-yl)-2H-indazol-2-yl)piperidine-2,6-dione

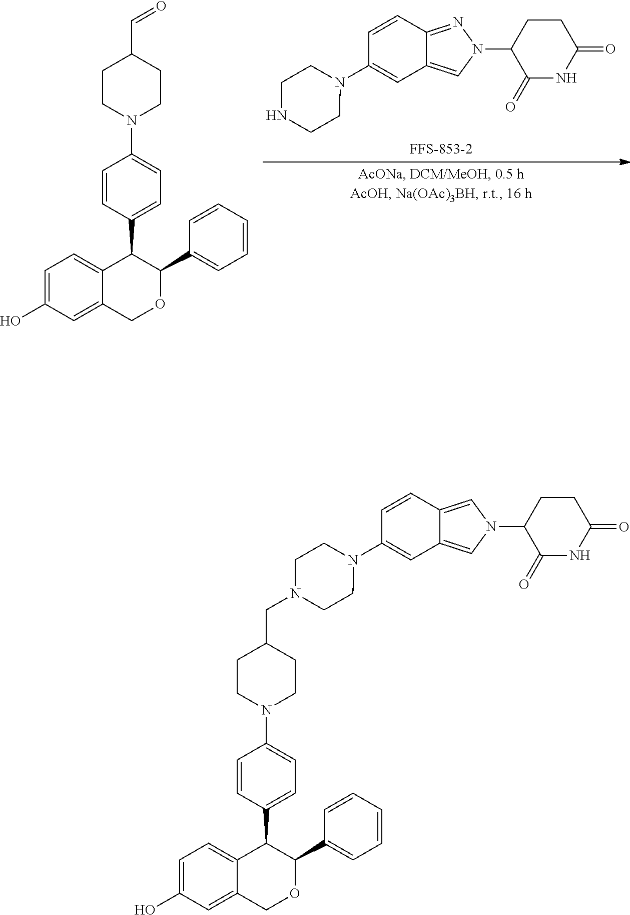 [SMILES: O=CC1CCN(c2ccc([C@@H]3c4ccc(O)cc4CO[C@@H]3c3ccccc3)cc2)CC1 ; O=C1CCC(n2cc3cc(N4CCNCC4)ccc3n2)C(=O)N1 ; O=C1CCC(n2cc3ccc(N4CCN(CC5CCN(c6ccc([C@@H]7c8ccc(O)cc8CO[C@@H]7c7ccccc7)cc6)CC5)CC4)cc3c2)C(=O)N1]

Step 1: A mixture of 1-(4-((3S,4R)-7-hydroxy-3-phenylisochroman-4-yl)phenyl)piperidine-4-carbaldehyde (100 mg, 93% purity, 1 Eq, 224 μmol), 3-(5-(piperazin-1-yl)-2H-indazol-2-yl)piperidine-2,6-dione (70.3 mg, 1 Eq, 224 μmol) and sodium acetate (92.0 mg, 5 Eq, 1.12 mmol) were added in DCM (2 mL) and MeOH (2 mL) at 25° C. for 0.5 hour, then added acetic acid (40.4 mg, 38.5 μL, 3 Eq, 673 μmol) and sodium triacetoxyborohydride (95.1 mg, 2 Eq, 449 μmol), then the mixture was stirred at 25° C. for 16 hour. The reaction was diluted with water (50 mL) and extracted with ethyl acetate (50 mL*2). The combined organic layers were concentrated to give crude product as a brown oil. The brown oil was purified by preparative high-performance liquid chromatography. Condition: water (FA)-ACN. Column: Boston Prime C18 150*30 mm*5 um. Begin B: 60%, End B: 90%. Gradient time (min) 7; 100% B hold time (min) 2, Flow rate (ml/min) 25. The aqueous layer was lyophilized to dryness to give 3-(5-(4-((1-(4-((3S,4R)-7-hydroxy-3-phenylisochroman-4-yl)phenyl)piperidin-4-yl)methyl)piperazin-1-yl)-2H-indazol-2-yl)piperidine-2,6-dione (22.3 mg, 29.9 μmol, 13.3% yield, 95.2% purity) as a white solid.

LCMS: calc. for C₄₃H₄₆N₆O₄:710.36, found: [M+H]⁺ 711.3.

HPLC: 95.2% purity at 220 nm.

¹H NMR (400 MHZ, DMSO-d₆) δ=11.07 (s, 1H), 9.29 (s, 1H), 7.94 (s, 1H), 7.48 (br d, J=9.2 Hz, 1H), 7.25 (brd, J=8.9 Hz, 1H), 7.13 (br d, J=7.5 Hz, 2H), 7.10-7.04 (m, 4H), 6.78 (d, J=8.0 Hz, 1H), 6.63-6.59 (m, 2H), 6.55 (br d, J=6.6 Hz, 4H), 5.75 (br dd, J=4.8, 11.7 Hz, 1H), 5.07 (br d, J=15.5 Hz, 1H), 5.00 (d, J=2.5 Hz, 1H), 4.90 (br d, J=15.0 Hz, 1H), 4.02 (br s, 1H), 3.50 (br d, J=11.4 Hz, 2H), 3.43-3.34 (m, 3H), 3.08 (br s, 4H), 2.90-2.80 (m, 1H), 2.72 (br s, 1H), 2.68 (br d, J=6.2 Hz, 1H), 2.49-2.43 (m, 3H), 2.27-2.15 (m, 3H), 1.74 (br d, J=12.3 Hz, 2H), 1.68-1.57 (m, 1H), 1.21-1.09 (m, 2H).

### Example 94. Preparation of (I-232) (S)-3-(5-(4-((1-(4-((3R,4R)-3-(cyclopentylmethyl)-7-hydroxyisochroman-4-yl)phenyl)piperidin-4-yl)methyl)piperazin-1-yl)-1-oxoisoindolin-2-yl)piperidine-2,6-dione and (I-233) (S)-3-(5-(4-((1-(4-((3S,4S)-3-(cyclopentylmethyl)-7-hydroxyisochroman-4-yl)phenyl)piperidin-4-yl)methyl)piperazin-1-yl)-1-oxoisoindolin-2-yl)piperidine-2,6-dione

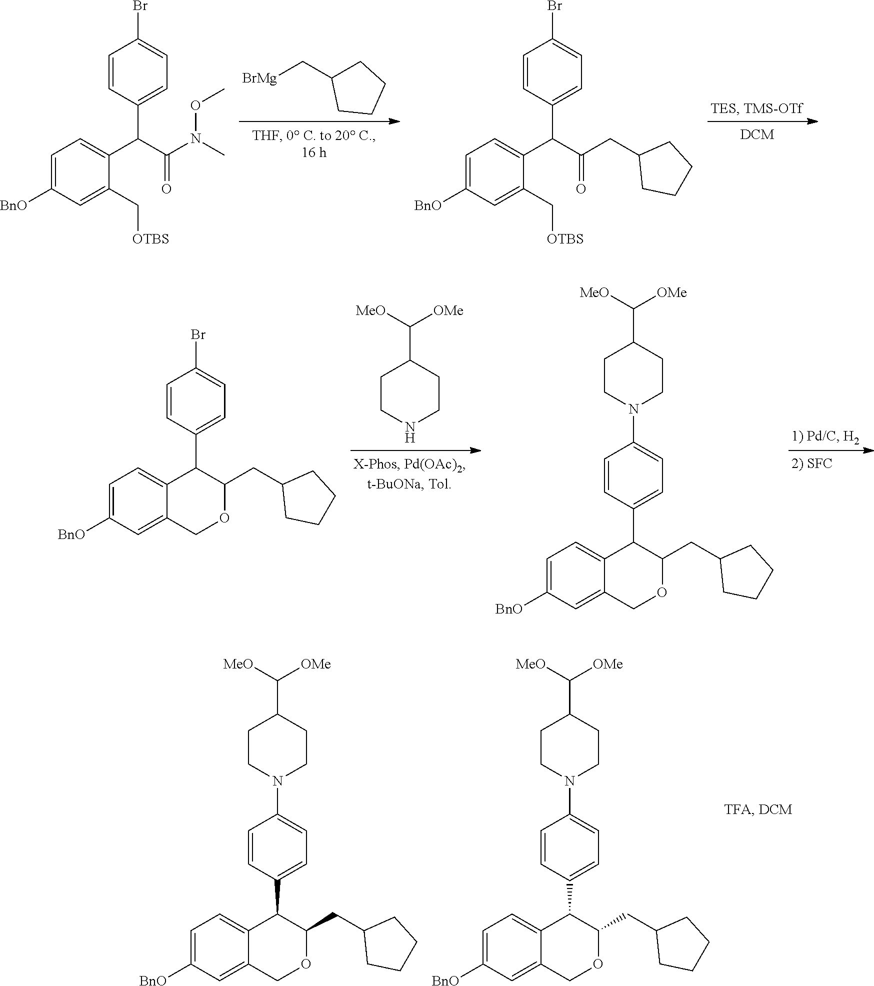

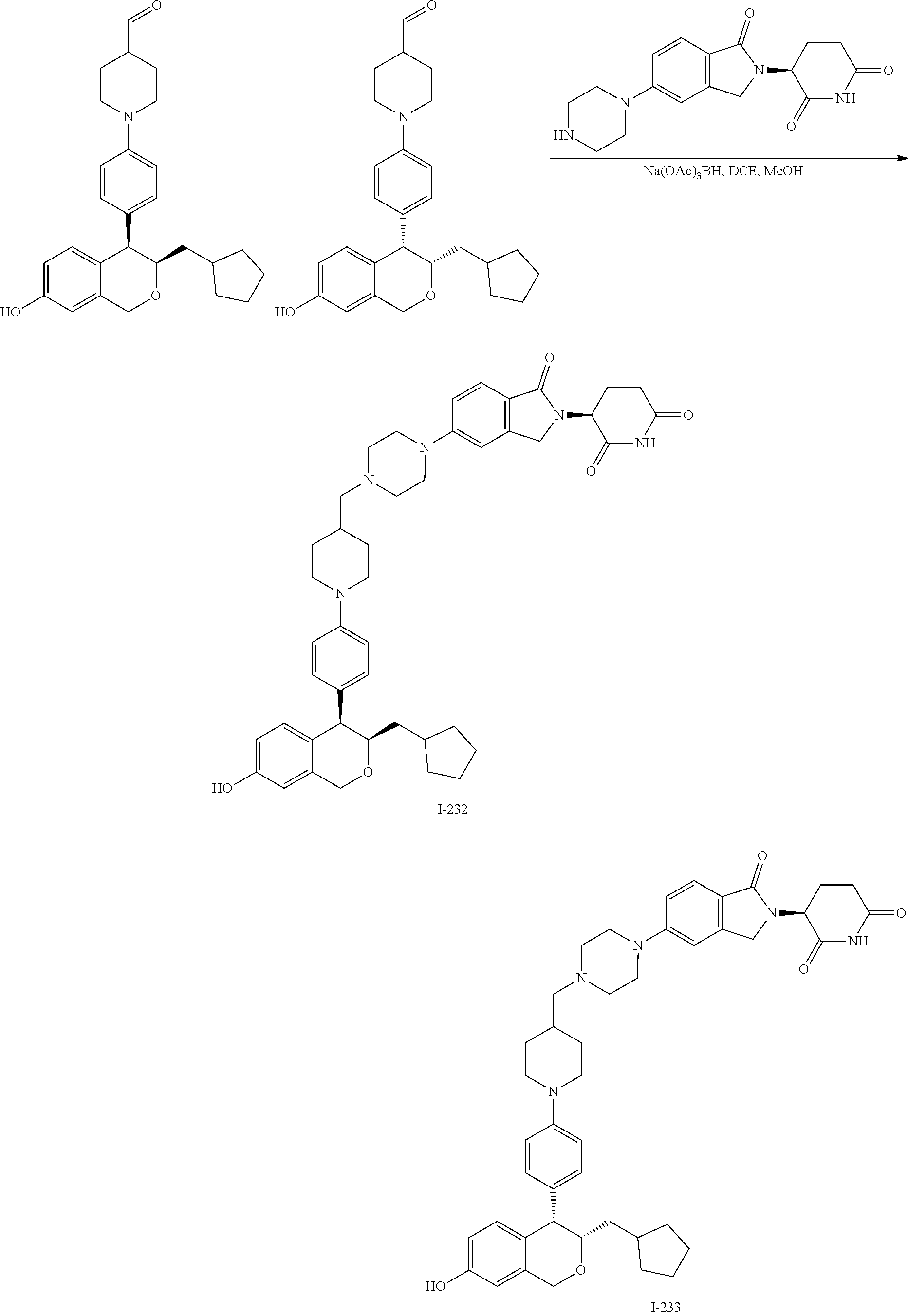

Step 1: To a solution of 2-[4-benzyloxy-2-[[tert-butyl(dimethyl)silyl]oxymethyl]phenyl]-2-(4-bromophenyl)-N-methoxy-N-methyl-acetamide (1 g, 1.71 mmol) in THF (3 mL) was added (cyclopentylmethyl) magnesium bromide (0.5 M, 13.68 mL) at 0° C. The mixture was stirred at 20° C. for 16 h under N₂ atmosphere. The reaction was quenched with sat. aq, NH₄Cl (40 mL) and extracted with EtOAc (30 mL×3). The combined organic layers were washed with brine (50 mL), dried over Na₂SO₄, filtered, and concentrated under reduced pressure to give a residue. The residue was purified by flash silica gel chromatography (ethyl acetate in petroleum ether=0% to 5%) to give a product of 1-[4-benzyloxy-2-[[tert-butyl(dimethyl)silyl]oxymethyl]phenyl]-1-(4-bromophenyl)-3-cyclopentyl-propan-2-one (850 mg, 73.6% yield) as a yellow gum.

¹HNMR (400 MHZ, CDCl₃): δ 7.46-7.30 (m, 7H), 7.08-6.94 (m, 4H), 6.88 (dd, J=2.7, 8.5 Hz, 1H), 5.39 (s, 1H), 5.07 (s, 2H), 4.68 (d, J=12.8 Hz, 1H), 4.50 (d, J=12.8 Hz, 1H), 2.55 (d, J=7.0 Hz, 2H), 2.32-2.21 (m, 1H), 1.89-1.75 (m, 2H), 1.58-1.48 (m, 4H), 1.13-0.96 (m, 2H), 0.92 (s, 9H), 0.06 (d, J=8.3 Hz, 6H)

Step 2: To a solution of 1-[4-benzyloxy-2-[[tert-butyl(dimethyl)silyl]oxymethyl]phenyl]-1-(4-bromophenyl)-3-cyclopentyl-propan-2-one (700 mg, 1.04 mmol) in DCM (10 mL) was added triethylsilane (180.82 mg, 1.56 mmol, 248.38 μL), the resulting mixture was cooled at −60° C. and trimethylsilyl trifluoromethanesulfonate (345.63 mg, 1.56 mmol, 281.00 μL) was added dropwise, and the resulting mixture was stirred at −60° C. for 1 h. The reaction mixture was quenched with sat. aq. NaHCO₃ (30 mL) and extracted with EtOAc (30 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 5%) to give the product of 7-benzyloxy-4-(4-bromophenyl)-3-(cyclopentylmethyl)isochromane (500 mg, 60.61% yield) as a yellow gum. LC-MS (ESI⁺) m/z: 477.0 (M+H)⁺.

Step 3: A mixture of 7-benzyloxy-4-(4-bromophenyl)-3-(cyclopentylmethyl)isochromane (500 mg, 628.36 μmol), 4-(dimethoxymethyl)piperidine (130.07 mg, 816.87 μmol), t-BuONa (120.77 mg, 1.26 mmol), Pd(OAc)₂ (14.11 mg, 62.84 μmol) and XPhos (59.91 mg, 125.67 μmol) in Tol (8 mL) was degassed and purged with N₂ for 3 times, and then the mixture was stirred at 90° C. for 16 h under N₂ atmosphere. The reaction was quenched with water (50 mL) and extracted with EtOAc (30 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 20%) to give the product of 1-[4-[7-benzyloxy-3-(cyclopentylmethyl)isochroman-4-yl]phenyl]-4-(dimethoxymethyl)piperidine (150 mg, 42.1% yield) as a yellow oil. LC-MS (ESI+) m/z: 556.4 (M+H)⁺.

Step 4: To a solution of Pd/C (150 mg, 10% purity) in THF (10 mL) was added 1-(4-(7-(benzyloxy)-3-(cyclopentylmethyl)isochroman-4-yl)phenyl)-4-(dimethoxymethyl)piperidine (150 mg, 270 μmol) under N₂. The suspension was degassed under vacuum and purged with H₂ several times. The mixture was stirred under H₂ (15 psi) at 25° C. for 8 h. The reaction mixture was filtered and the filter was concentrated to yield a residue which was purified by flash silica gel chromatography (ethyl acetate in petroleum ether=0% to 30%) to give a residue. The residue was separated by chiral SFC (column: DAICEL CHIRALPAK ID (250 mm*30 mm, 10 um); mobile phase: [MeOH-ACN]B %: 50%, isocratic elution mode, peak 1: 0.697 min, peak 2:0.984 min) to yield Peak 1 and Peak 2. Peak 1 was concentrated under reduced pressure to give the product of (3R,4R)-3-(cyclopentylmethyl)-4-(4-(4-(dimethoxymethyl)piperidin-1-yl)phenyl)isochroman-7-ol (40 mg, 32% yield, SFC: R; =0.697) as a white solid. LC-MS (ESI⁺) m/z: 466.3 (M+H)⁺. Peak 2 was concentrated under reduced pressure to give the product of (3S,4S)-3-(cyclopentylmethyl)-4-(4-(4-(dimethoxymethyl)piperidin-1-yl)phenyl)isochroman-7-ol (40 mg, 32% yield, SFC: Rt=0.984) as a white solid. LC-MS (ESI⁺) m/z: 466.3 (M+H)⁺.

Step 5: To a solution of (3R,4R)-3-(cyclopentylmethyl)-4-(4-(4-(dimethoxymethyl)piperidin-1-yl)phenyl)isochroman-7-ol (40 mg, 86 μmol) in DCM (2 mL) was added TFA (0.8 mL, 0.01 mol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give 1-(4-((3R,4R)-3-(cyclopentylmethyl)-7-hydroxyisochroman-4-yl)phenyl)piperidine-4-carbaldehyde (36 mg, crude) as a yellow oil. LC-MS (ESI+) m/z: 420.3 (M+H)⁺.

Step 6: To a solution of (3S,4S)-3-(cyclopentylmethyl)-4-(4-(4-(dimethoxymethyl)piperidin-1-yl)phenyl)isochroman-7-ol (40 mg, 86 μmol) in DCM (2 mL) was added TFA (0.8 mL, 0.01 mol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give 1-(4-((3S,4S)-3-(cyclopentylmethyl)-7-hydroxyisochroman-4-yl)phenyl)piperidine-4-carbaldehyde (36 mg, crude) as a yellow oil. LC-MS (ESI+) m/z: 420.3 (M+H)⁺.

Step 7: To a solution of 1-(4-((3R,4R)-3-(cyclopentylmethyl)-7-hydroxyisochroman-4-yl)phenyl)piperidine-4-carbaldehyde (36 mg, 86 μmol) in DCE (10 mL) and MeOH (1 mL) was added (S)-3-(1-oxo-5-(piperazin-1-yl)isoindolin-2-yl)piperidine-2,6-dione (28 mg, 86 μmol) and stirred at 20° C. for 10 min. Then NaBH(OAc)₃ (55 mg, 0.26 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; B %: 57%-87%, 11 min), followed by lyophilization to give the product of (S)-3-(5-(4-((1-(4-((3R,4R)-3-(cyclopentylmethyl)-7-hydroxyisochroman-4-yl)phenyl)piperidin-4-yl)methyl)piperazin-1-yl)-1-oxoisoindolin-2-yl)piperidine-2,6-dione (27 mg, 43% yield) as a white solid. LC-MS (ESI+) m/z: 732.4 (M+H)⁺.

LCMS: calc. for C₄₄H₅₃N₅O₅:731.94, found: [M+H]⁺ 732.4.

HPLC: 100.0% purity at 254 nm.

¹HNMR (400 MHz, DMSO-d₆): § 10.95 (s, 1H), 9.20 (s, 1H), 7.52 (d, J=8.6 Hz, 1H), 7.11-7.01 (m, 2H), 6.95 (d, J=8.6 Hz, 2H), 6.77 (d, J=8.7 Hz, 2H), 6.71 (d, J=8.3 Hz, 1H), 6.54-6.47 (m, 1H), 6.45 (s, 1H), 5.04 (dd, J=5.2, 13.2 Hz, 1H), 4.88-4.62 (m, 2H), 4.38-4.14 (m, 2H), 3.79-3.70 (m, 1H), 3.65-3.54 (m, 3H), 3.32-3.24 (m, 8H), 2.98-2.83 (m, 1H), 2.65-2.53 (m, 4H), 2.21 (d, J=6.9 Hz, 2H), 2.02-1.86 (m, 2H), 1.80 (d, J=12.5 Hz, 2H), 1.67 (d, J=4.2 Hz, 3H), 1.58-1.37 (m, 4H), 1.28-1.12 (m, 3H), 1.11-0.92 (m, 3H)

Step 8: To a solution of 1-(4-((3S,4S)-3-(cyclopentylmethyl)-7-hydroxyisochroman-4-yl)phenyl)piperidine-4-carbaldehyde (36 mg, 86 μmol) in DCE (10 mL) and MeOH (1 mL) was added (S)-3-(1-oxo-5-(piperazin-1-yl)isoindolin-2-yl)piperidine-2,6-dione (28 mg, 86 μmol) and stirred at 20° C. for 10 min. Then NaBH(OAc)₃ (55 mg, 0.26 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; B %: 57%-87%, 11 min), followed by lyophilization to give the product of (S)-3-(5-(4-((1-(4-((3S,4S)-3-(cyclopentylmethyl)-7-hydroxyisochroman-4-yl)phenyl)piperidin-4-yl)methyl)piperazin-1-yl)-1-oxoisoindolin-2-yl)piperidine-2,6-dione (23 mg, 37%, yield) as a white solid. LC-MS (ESI+) m/z: 732.5 (M+H)⁺.

LCMS: calc. for C₄₄H₅₃N₅O₅:731.94, found: [M+H]⁺ 732.5.

HPLC: 100.0% purity at 254 nm.

¹HNMR (400 MHz, DMSO-d₆): § 10.95 (s, 1H), 9.20 (s, 1H), 7.52 (d, J=8.6 Hz, 1H), 7.09-7.03 (m, 2H), 6.95 (d, J=8.6 Hz, 2H), 6.77 (d, J=8.8 Hz, 2H), 6.71 (d, J=8.3 Hz, 1H), 6.53-6.48 (m, 1H), 6.45 (s, 1H), 5.04 (dd, J=4.9, 13.2 Hz, 1H), 4.87-4.64 (m, 2H), 4.38-4.16 (m, 2H), 3.78-3.70 (m, 1H), 3.62 (s, 3H), 3.30-3.25 (m, 8H), 2.98-2.81 (m, 1H), 2.59 (d, J=10.0 Hz, 4H), 2.21 (d, J=6.9 Hz, 2H), 2.02-1.86 (m, 2H), 1.80 (d, J=11.2 Hz, 2H), 1.67 (d, J=3.7 Hz, 3H), 1.56-1.39 (m, 4H), 1.26-1.12 (m, 3H), 1.10-0.89 (m, 3H).

### Example 95. Preparation of (I-355) (S)-3-(5-(4-((1-(4-((3S,4R)-3-(3-fluoro-4-methylphenyl)-7-hydroxyisochroman-4-yl)phenyl)piperidin-4-yl)methyl)piperazin-1-yl)-1-oxoisoindolin-2-yl)piperidine-2,6-dione and (I-356) (S)-3-(5-(4-((1-(4-((3R,4S)-3-(3-fluoro-4-methylphenyl)-7-hydroxyisochroman-4-yl)phenyl)piperidin-4-yl)methyl)piperazin-1-yl)-1-oxoisoindolin-2-yl)piperidine-2,6-dione

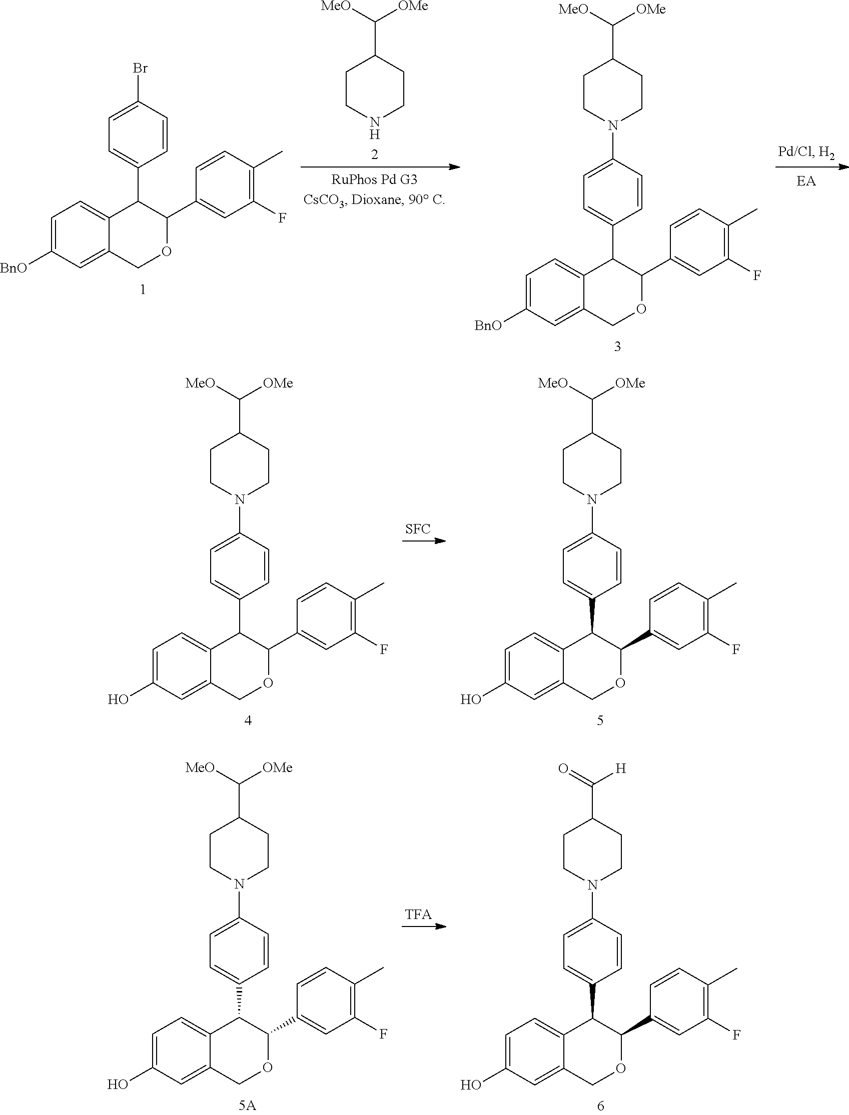

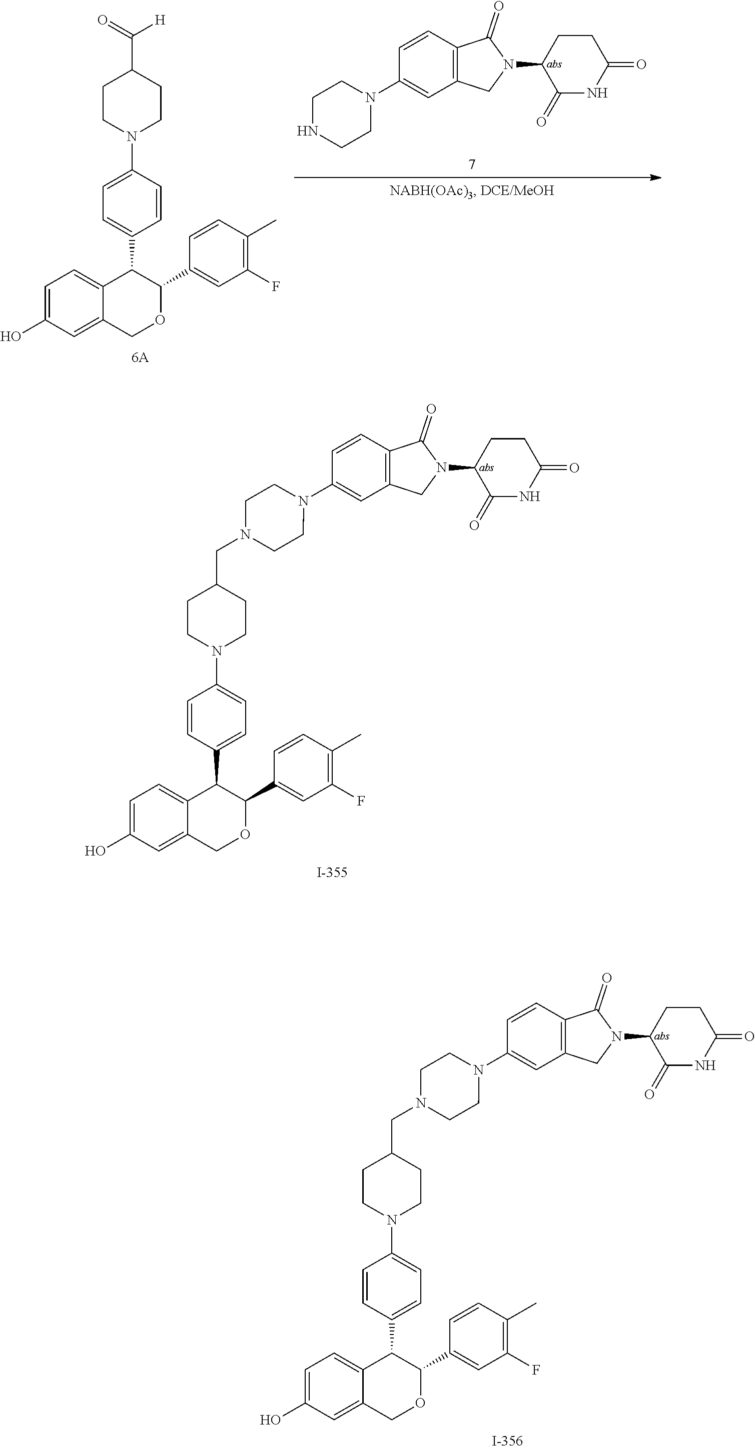 [SMILES: Cc1ccc([C@@H]2OCc3cc(O)ccc3[C@@H]2c2ccc(N3CCC(C=O)CC3)cc2)cc1F ; O=C1CC[C@H](N2Cc3cc(N4CCNCC4)ccc3C2=O)C(=O)N1 ; Cc1ccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F ; Cc1ccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F]

Step 1: To a solution of compound 1 (250.00 mg, 496.62 μmol, 1.0 eq.), compound 2 (158.15 mg, 993.24 μmol, 2.0 eq.), Cs₂CO₃ (404.52 mg, 1.24 mmol, 2.5 eq.) and RuPhos Pd G3 (41.54 mg, 49.66 μmol, 0.1 eq.) in dioxane (5 mL) was bubbled with N₂ for 1 min. Then the mixture was stirred at 90° C. for 5 hours. LCMS showed the starting material was consumed completely and a major peak with desired MS was detected. The reaction mixture was concentrated to get a residue. The residue was purified by column chromatography (SiO₂, Petroleum ether/Ethyl acetate=1/0 to 3/1) to give compound 3 (300.00 mg, 52.96% yield) as a yellow solid.

LC-MS (ESI⁺) m/z: 582.3 (M+H)⁺.

¹H NMR (400 MHZ, DMSO-d₆) δ ppm 7.49-7.30 (m, 5H), 7.06 (t, J=8.0 Hz, 1H), 6.92-6.72 (m, 5H), 6.68-6.55 (m, 4H), 5.15-4.91 (m, 5H), 4.05 (s, 2H), 3.57-3.48 (m, 2H), 3.24 (s, 6H), 2.46-2.32 (m, 2H), 2.12 (s, 3H), 1.70-1.57 (m, 3H), 1.30-1.20 (m, 2H).

Step 2: To a solution of compound 1 (250.00 mg, 496.62 μmol, 1.0 eq.), compound 2 (158.15 mg, 993.24 μmol, 2 eq.), Cs₂CO₃ (404.52 mg, 1.24 mmol, 2.5 eq.) and RuPhos Pd G3 (41.54 mg, 49.66 μmol, 0.1 eq.) in dioxane (5 mL) was bubbled with N₂ for 1 min. Then the mixture was stirred at 90° C. for 5 hours. LCMS showed the starting material was consumed completely and a major peak with desired MS was detected. The reaction mixture was concentrated to get a residue. The residue was purified by column chromatography (SiO₂, Petroleum ether/Ethyl acetate=1/0 to 3/1) to give compound 2 (300.00 mg, 72.69% yield) as a yellow solid.

LC-MS (ESI⁺) m/z: 582.3 (M+H)⁺

¹H NMR (400 MHZ, DMSO-d₆) δ=7.49-7.26 (m, 5H), 7.06 (t, J=8.0 Hz, 1H), 6.94-6.70 (m, 5H), 6.68-6.54 (m, 4H), 5.16-4.91 (m, 5H), 4.12-4.04 (m, 2H), 3.61-3.45 (m, 2H), 3.28-3.20 (m, 6H), 2.47-2.39 (m, 2H), 2.16-2.08 (m, 3H), 1.71-1.58 (m, 3H), 1.31-1.20 (m, 2H)

Step 3: To a solution of compound 3 (300.00 mg, 515.72 μmol, 1.0 eq.) in EA (20 mL) was added Pd/C (548.83 mg, 515.72 μmol, 10% purity, 1.0 eq.) under N₂ atmosphere. The suspension was degassed and purged with H₂ for 3 times. The mixture was stirred under H₂ (15 Psi) at 15° C. for 4 hr. LCMS showed that the starting material was consumed and the desired product was detected. The reaction mixture was concentrated to get a residue. The residue was purified by prep-HPLC (column: Boston Green ODS 150×30 mm×5 um; mobile phase: [water (FA)-ACN]; gradient: 25%-60% B over 14 min.) to give compound 4 (130.00 mg, 51.28% yield) as a yellow solid.

LC-MS (ESI⁺) m/z: 492.2 (M+H)⁺.

Step 4: To a solution of compound 3 (298.00 mg, 512.28 μmol, 1.0 eq.) in EA (20 mL) was added Pd/C (545.17 mg, 512.28 μmol, 10% purity, 1.0 eq.) under N₂ atmosphere. The suspension was degassed and purged with H₂ for 3 times. The mixture was stirred under H₂ (15 Psi) at 15° C. for 4 hr. LCMS showed that the starting material was consumed and the desired product was detected. The reaction mixture was concentrated to get a residue. The residue was purified by prep-HPLC (column: Boston Green ODS 150×30 mm×5 um; mobile phase: [water (FA)-ACN]; gradient: 25%-60% B over 14 min.) to give compound 4 (130.00 mg, 50.07% yield) as a yellow solid.

LC-MS (ESI⁺) m/z: 492.2 (M+H)⁺.

Step 5: Compound 4 (200.00 mg, 407 μmol, 1.0 eq.) was purified by SFC (Column: Chiralpak AD-3 150; A4.6 mm I.D, 3 um Mobile phase: 40% of iso-propanol (0.05% DEA) in CO₂, Flow rate: 2.5 mL/min, RT: 1.682, 3.608) to give compound 5 (80.00 mg, 40% yield) as a white solid and compound 5A (50.00 mg, 25% yield) as a white solid.

Compound 5: LC-MS (ESI⁺) m/z: 492.2 (M+H)⁺.

Compound 5A: LC-MS (ESI⁺) m/z: 492.3 (M+H)⁺.

Step 6: To a solution of compound 5 (100.00 mg, 203 μmol, 1.0 eq.) in DCM (3 mL) was added TFA (1 mL) at 20° C. The mixture was stirred at 20° C. for 2 hour. LCMS showed the starting material was consumed completely and a major peak with desired MS was detected. Water (10 mL) and aq. NaHCO₃ (15×2 mL) was added and the mixture was extracted with DCM (20 mL×3). The organics were washed with brine (20 mL), dried over Na₂SO₄, filtered and concentrated to give compound 6 (80.00 mg, 87% yield) as a white solid.

LC-MS (ESI⁺) m/z: 446.3 (M+H)⁺.

Step 7: To a solution of compound 5A (50.00 mg, 0.10 mmol, 1.0 eq.) in DCM (1.5 mL) was added TFA (0.5 mL) at 20° C. The mixture was stirred at 20° C. for 2 hour. LCMS showed the starting material was consumed completely and a major peak with desired MS was detected. Water (10 mL) and aq. NaHCO₃ (15×2 mL) was added and the mixture was extracted with DCM (20 mL×3). The organics were washed with brine (20 mL), dried over Na₂SO₄, filtered and concentrated to give compound 6A (42.00 mg, 92% yield) as a yellow solid.

LC-MS (ESI⁺) m/z: 446.3 (M+H)⁺.

Step 8: To a solution of compound 6 (80.00 mg, 0.18 mmol, 1.0 eq.) and compound 7 (65.00 mg, 0.20 mmol, 1.1 eq.) in DCE (3 mL) and MeOH (1 mL) was added NaBH(OAc)₃ (0.19 g, 0.13 mL, 0.90 mmol, 5.0 eq.) at 20° C. Then the mixture was stirred at 20° C. for 2 hour. LCMS showed the starting material was consumed completely and a major peak with desired MS was detected. The mixture was concentrated to get the crude, which was purified by prep-HPLC (column: Phenomenex Gemini NX 150×30 mm, 5 um; mobile phase: [water(NH₄HCO₃)-ACN]; gradient: 53%-83% B over 11 min.) to give (S)-3-(5-(4-((1-(4-((3S,4R)-3-(3-fluoro-4-methylphenyl)-7-hydroxyisochroman-4-yl)phenyl)piperidin-4-yl)methyl)piperazin-1-yl)-1-oxoisoindolin-2-yl)piperidine-2,6-dione (56.7 mg, 41% yield) as a white solid.

LC-MS (ESI⁺) m/z: 758.4 (M+H)⁺.

LCMS: calc. for C₄₅H₄₈FN₅O₅: 757.91, found [M+H]⁺ 758.4.

HPLC: 99.11% purity at 220 nm

¹H NMR (400 MHZ, DMSO-d₆) δ ppm 10.94 (s, 1H) 9.28 (s, 1H) 7.51 (d, J=8.46 Hz, 1H) 7.03-7.08 (m, 3H) 6.86 (d, J=8.11 Hz, 1H) 6.71-6.79 (m, 2H) 6.63-6.67 (m, 2H) 6.52-6.60 (m, 4H) 5.01-5.09 (m, 2H) 4.97 (d, J=2.26 Hz, 1H) 4.89 (br d, J=15.26 Hz, 1H) 4.17-4.35 (m, 2H) 3.99-4.07 (m, 1H) 3.52 (br d, J=11.92 Hz, 2H) 3.27 (br s, 4H) 2.84-2.95 (m, 1H) 2.51-2.69 (m, 4H) 2.31-2.48 (m, 4H) 2.18 (br d, J=7.03 Hz, 2H) 2.12 (s, 3H) 1.92-1.99 (m, 1H) 1.59-1.77 (m, 3H) 1.09-1.20 (m, 2H)

Step 9: To a solution of compound 6A (42.00 mg, 94 μmol, 1.0 eq.) and compound 7 (34.00 mg, 0.10 mmol, 1.1 eq.) in DCE (3 mL) and MeOH (1 mL) was added NaBH(OAc)₃ (0.10 g, 70
L, 0.47 mmol, 5.0 eq.) at 20° C. Then the mixture was stirred at 20° C. for 2 hour. LCMS showed the starting material was consumed completely and a major peak with desired MS was detected. The mixture was concentrated to get the crude, which was purified by prep-HPLC (column: Phenomenex Gemini NX 150×30 mm, 5 μm; mobile phase: [water(NH₄HCO₃)-ACN]; gradient: 53%-83% B over 11 min) to give (S)-3-(5-(4-((1-(4-((3R,4S)-3-(3-fluoro-4-methylphenyl)-7-hydroxyisochroman-4-yl)phenyl)piperidin-4-yl)methyl)piperazin-1-yl)-1-oxoisoindolin-2-yl)piperidine-2,6-dione (47.6 mg, 66% yield) as a white solid.

LC-MS (ESI⁺) m/z: 758.4 (M+H)⁺.

LCMS: calc. for C₄₅H₄₈FN₅O₅: 757.91, found [M+H]⁺ 758.4.

HPLC: 99.27% purity at 220 nm

¹H NMR (400 MHZ, DMSO-d₆) δ ppm 10.90-10.98 (m, 1H) 9.28 (s, 1H) 7.51 (d, J=8.46 Hz, 1H) 7.03-7.09 (m, 3H) 6.86 (d, J=7.39 Hz, 1H) 6.71-6.79 (m, 2H) 6.63-6.67 (m, 2H) 6.52-6.60 (m, 4H) 5.01-5.09 (m, 2H) 4.97 (d, J=2.74 Hz, 1H) 4.89 (br d, J=15.14 Hz, 1H) 4.17-4.35 (m, 2H) 4.03 (d, J=2.86 Hz, 1H) 3.52 (br d, J=10.37 Hz, 2H) 3.27 (br s, 4H) 2.84-2.95 (m, 1H) 2.51-2.74 (m, 4H) 2.25-2.48 (m, 4H) 2.18 (br d, J=7.03 Hz, 2H) 2.12 (s, 3H) 1.91-1.99 (m, 1H) 1.60-1.78 (m, 3H) 1.09-1.20 (m, 2H).

### Example 96. Preparation of (I-363) (S)-3-(5-(4-((1-(4-((3S,4R)-3-(bicyclo[4.2.0]octa-1(6),2,4-trien-3-yl)-7-hydroxyisochroman-4-yl)phenyl)piperidin-4-yl)methyl)piperazin-1-yl)-1-oxoisoindolin-2-yl)piperidine-2,6-dione

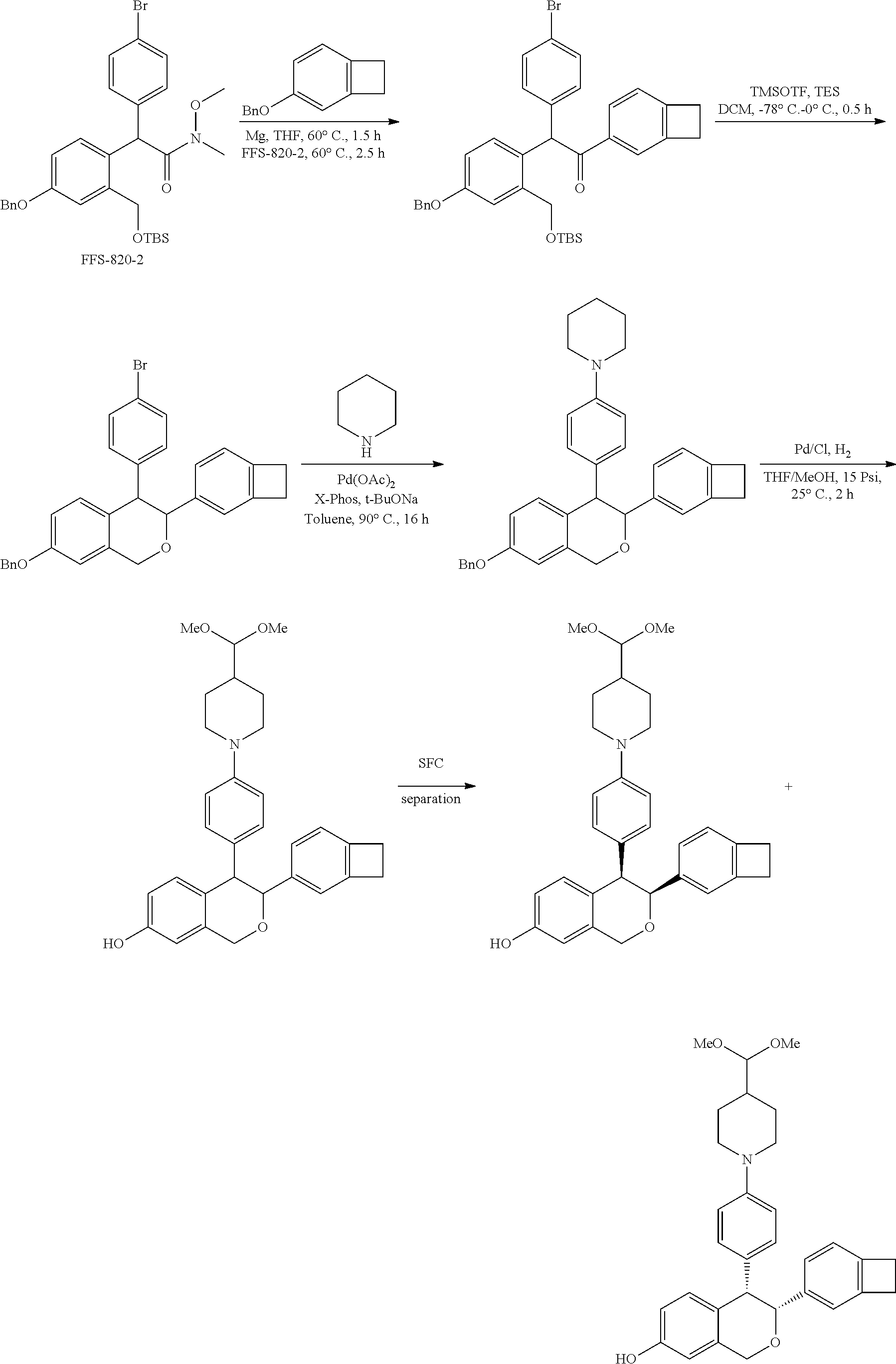

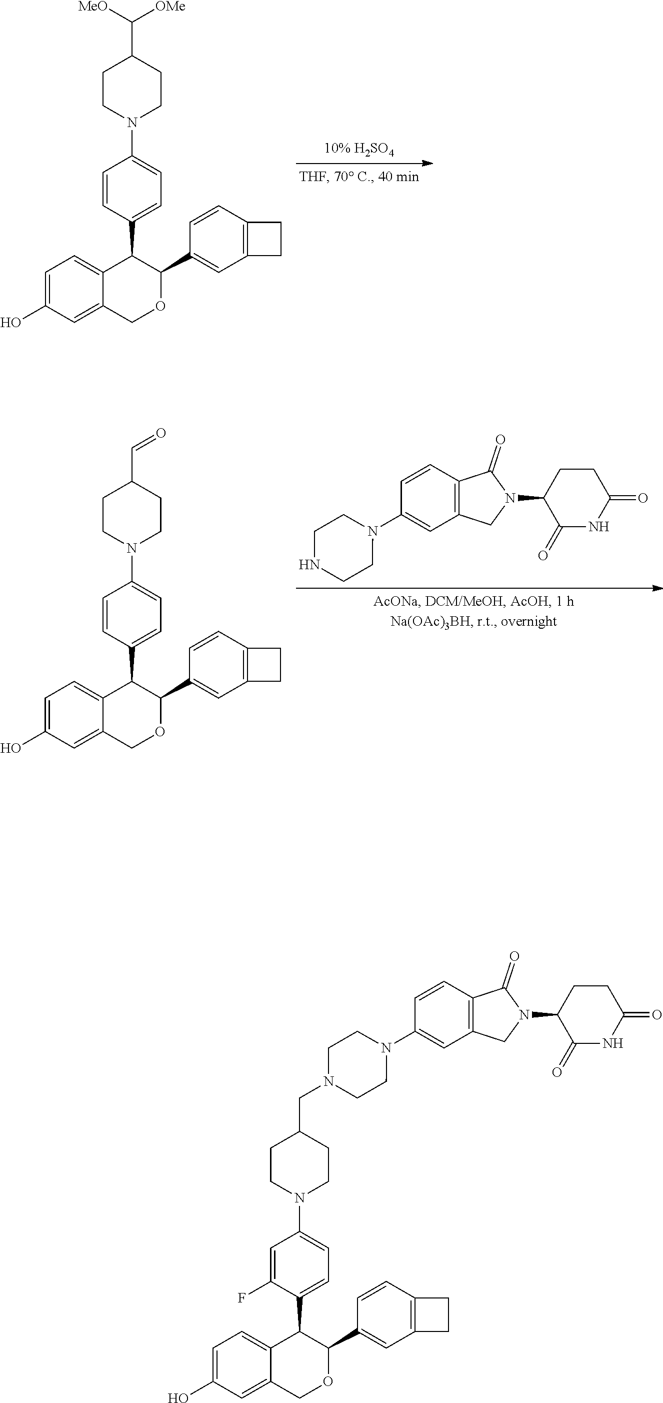 [SMILES: COC(OC)C1CCN(c2ccc([C@@H]3c4ccc(O)cc4CO[C@@H]3c3ccc4c(c3)CC4)cc2)CC1 ; O=CC1CCN(c2ccc([C@@H]3c4ccc(O)cc4CO[C@@H]3c3ccc4c(c3)CC4)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccc8c(c7)CC8)c(F)c6)CC5)CC4)ccc3C2=O)C(=O)N1]

Step 1: A dried three-neck flask equipped with thermometer and condenser was charged with magnesium (312 mg, 5 Eq, 12.8 mmol) and a catalyst diiodine (651 mg, 1 Eq, 2.57 mmol)). The mixture was refluxed 70° C. for 30 min under N₂, then added by a solution of 3-bromobicyclo [4.2.0]octa-1, 3, 5-triene (2.4 g, 5 Eq, 12.8 mmol) in THF (70 mL) dropwise. The above reaction mixture was refluxed at 70° C. for 1.5 hour. The above reaction mixture was added into a solution of 2-(4-(benzyloxy)-2-(((tert-butyldimethylsilyl)oxy)methyl)phenyl)-2-(4-bromophenyl)-N-methoxy-N-methylacetamide (1.5 g, 1 Eq, 2.57 mmol) in THF (30 mL) at 25° C. under N₂ and stirred at 25° C. under N₂ for 16 hour. The reaction solution was quenched by saturated aq NH₄Cl (50 mL). Then the mixture was added water (50 mL) and the mixture was extracted with dichloromethane (100 mL×3). The combined organic layers were washed with brine (100 mL), dried over anhydrous Na₂SO₄, filtered and concentrated to dryness in vacuum to give 2-(4-(benzyloxy)-2-(((tert-butyldimethylsilyl)oxy)methyl)phenyl)-1-(bicyclo[4.2.0]octa-1,3,5-trien-3-yl)-2-(4-bromophenyl) ethan-1-one (1.2 g, 1.7 mmol, 68% yield, 96% purity) as a green oil. LC-MS (ESI⁺) m/z: 497.0 (M−129)⁺.

Step 2: To a solution of 2-(4-(benzyloxy)-2-(((tert-butyldimethylsilyl)oxy)methyl)phenyl)-1-(bicyclo[4.2.0]octa-1(6),2,4-trien-3-yl)-2-(4-bromophenyl) ethan-1-one (400 mg, 96% purity, 1 Eq, 618 μmol) and triethylsilane (108 mg, 148 μL, 1.5 Eq, 927 μmol) were added in DCM (5 mL), the resulting mixture was cooled at −78° C. and trimethylsilyl triflate (206 mg, 171 μL, 1.5 Eq, 927 μmol) was added, and the resulting mixture was allowed to stirred for 30 min at this temperature. The reaction mixture was quenched with saturated aq. NaHCO₃ (10 mL). The reaction was diluted with water (50 mL) and extracted with dichloromethane (50 mL*2). The combined organic layers were washed with brine (50 mL), dried over anhydrous Na₂SO₄, filtered and concentrated to dryness in vacuum to give a yellow oil. The yellow oil was purified by flash column (ISCOR; 20 g SepaFlash® Silica Flash Column, petroleum ether/ethyl acetate from 100/0 to 90/10, @ 40 mL/min) and the fraction was concentrated in vacuum to give 7-(benzyloxy)-3-(bicyclo[4.2.0]octa-1(6),2,4-trien-3-yl)-4-(4-bromophenyl)isochromane (270 mg, 0.52 mmol, 83% yield, 89% purity) as a colorless oil.

Step 3: A mixture of 7-(benzyloxy)-3-(bicyclo[4.2.0]octa-1(6),2,4-trien-3-yl)-4-(4-bromophenyl)isochromane (220 mg, 89% purity, 1 Eq, 396 μmol), 2-(Dicyclohexylphosphanyl)-2′,4′,6′-tris(isopropyl) biphenyl (38 mg, 0.2 Eq, 79.1 μmol), palladium diacetate (13 mg, 0.15 Eq, 59.3 μmol), 4-(dimethoxymethyl)piperidine (95 mg, 99 μL, 1.5 Eq, 593 μmol), and sodium 2-methylpropan-2-olate (57 mg, 64 μL, 1.5 Eq, 593 μmol) were added in toluene (5 mL) and heated to 90° C. for 16 hours under N₂ atmosphere. TLC (petroleum ether/ethyl acetate=5/1, R_{f}=0.4) showed a new spot was formed. The reaction was diluted with H₂O (100 mL) and extracted with ethyl acetate (50 mL*2). The combined extracts was dried over anhydrous Na₂SO₄, filtered and concentrated to dryness in vacuum to give a residue. The residue was subjected to column chromatography (ISCOR; 20 g SepaFlash® Silica Flash Column, petroleum ether/ethyl acetate from 100/0 to 80/20, @ 40 mL/min). The pure fractions were collected and concentrated to dryness in vacuum to give 1-(4-(7-(benzyloxy)-3-(bicyclo[4.2.0]octa-1(6), 2, 4-trien-3-yl)isochroman-4-yl)phenyl)-4-(dimethoxymethyl)piperidine (130 mg, 226 μmol, 57.1% yield) as a white solid. LC-MS (ESI⁺) m/z: 576.1 (M+H)⁺

Step 4: A mixture of 1-(4-(7-(benzyloxy)-3-(bicyclo[4.2.0]octa-1(6), 2,4-trien-3-yl)isochroman-4-yl)phenyl)-4-(dimethoxymethyl)piperidine (130 mg, 1 Eq, 226 μmol) and Pd/C (130 mg, 10% Wt) were added in THF (2.5 mL) and MeOH (2.5 mL). Then the reaction was degassed and purged with H₂ for 3 times, and then the mixture was stirred at 25° C. for 2 hour under H₂ atmosphere (15 psi). The reaction was filtered and concentrated to dryness in vacuum to give 3-(bicyclo[4.2.0]octa-1(6),2,4-trien-3-yl)-4-(4-(4-(dimethoxymethyl)piperidin-1-yl)phenyl) isochroman-7-ol (120 mg, 225 μmol, 99.7% yield, 91.08% purity) as a white solid. LC-MS (ESI⁺) m/z: 486.3 (M+H)⁺.

Step 5: The white solid was purified by SFC. Column: DAICEL CHIRALPAK IG (250 mm*30 mm, 10 um); Condition: CO₂-EtOH (0.1% NH₃H₂O); at the beginning: B (50%); at the end: B (50%); Flow Rate (ml/min) 80. The pure fractions were collected respectively and the solvents were evaporated under vacuum to give (3S,4R)-3-(bicyclo[4.2.0]octa-1(6),2,4-trien-3-yl)-4-(4-(4-(dimethoxymethyl)piperidin-1-yl)phenyl)isochroman-7-ol (50 mg, 0.10 mmol, 44% yield, 96.7% purity) as a white solid and (3R,4S)-3-(bicyclo[4.2.0]octa-1(6),2,4-trien-3-yl)-4-(4-(4-(dimethoxymethyl)piperidin-1-yl)phenyl)isochroman-7-ol (54 mg, 0.11 mmol, 49% yield, 100% purity) as a white solid. LC-MS (ESI⁺) m/z: 486.4 (M+H)⁺.

Step 6: To a solution of (3S,4R)-3-(bicyclo[4.2.0]octa-1(6),2,4-trien-3-yl)-4-(4-(4-(dimethoxymethyl)piperidin-1-yl)phenyl)isochroman-7-ol (50 mg, 96.7% purity, 1 Eq, 0.10 mmol) in THF (5 mL) was added 10% sulfuric acid (5 mL) at 0° C. The mixture was stirred at 70° C. for 40 min. The mixture was adjusted to pH=8 by adding saturated aq. NaHCO₃ (10 mL) at 0° C. for 10 min. The reaction mixture was diluted with 50 mL H₂O and extracted with 50 mL ethyl acetate. The organic layers were washed with 50 mL brine and dried over anhydrous Na₂SO₄. The organic layers were filtered and concentrated under reduced pressure to give crude 1-(4-((3S,4R)-3-(bicyclo[4.2.0]octa-1(6),2,4-trien-3-yl)-7-hydroxyisochroman-4-yl)phenyl) piperidine-4-carbaldehyde (40 mg, 90 μmol, 90% yield, 98.6% purity) as a brown oil. LC-MS (ESI⁺) m/z: 458.1 (M+18)⁺

Step 7: A mixture of 1-(4-((3S,4R)-3-(bicyclo[4.2.0]octa-1(6),2,4-trien-3-yl)-7-hydroxyisochroman-4-yl)phenyl)piperidine-4-carbaldehyde (65 mg, 98.6% purity, 1 Eq, 0.15 mmol), (S)-3-(1-oxo-5-(piperazin-1-yl)isoindolin-2-yl)piperidine-2,6-dione hydrochloride (53 mg, 1 Eq, 0.15 mmol) and sodium acetate (60 mg, 5 Eq, 0.73 mmol) were added in DCM (2 mL) and MeOH (2 mL) at 25° C. for 0.5 hour, then added acetic acid (26 mg, 25 μL., 3 Eq, 0.44 mmol) and sodium triacetoxyborohydride (62 mg, 2 Eq, 0.29 mmol), and then the mixture was stirred at 25° C. for 16 hour. The reaction was diluted with water (50 mL) and extracted with ethyl acetate (50 mL*2). The combined organic layers were concentrated to give crude product as a brown oil. The brown oil was purified by preparative high-performance liquid chromatography. Condition: water (FA)-ACN. Column: Welch Xtimate C18 150*30 mm*5 um. Begin B: 20%, End B: 50%. Gradient time (min) 7; 100% B hold Time (min) 1.7, Flow rate (ml/min) 25. The pure fractions were collected, and the solvent was evaporated under vacuum. The aqueous layer was lyophilized to dryness to give (S)-3-(5-(4-((1-(4-((3S,4R)-3-(bicyclo[4.2.0]octa-1(6),2,4-trien-3-yl)-7-hydroxyisochroman-4-yl)phenyl)piperidin-4-yl)methyl)piperazin-1-yl)-1-oxoisoindolin-2-yl)piperidine-2,6-dione (38.6 mg, 49.9 μmol, 34% yield, 97.15% purity) as a white solid.

LCMS: calc. for C₄₆H₄₉N₅O₅:751.37, found: [M+H]⁺ 752.3.

HPLC: 97.15% purity at 220 nm.

¹H NMR (400 MHZ, DMSO-d₆) δ=10.92 (s, 1H), 9.24 (br s, 1H), 8.16 (s, 0.203H), 7.51 (d, J=8.6 Hz, 1H), 7.07-7.03 (m, 2H), 6.92-6.88 (m, 1H), 6.86-6.81 (m, 1H), 6.79-6.73 (m, 2H), 6.67-6.62 (m, 2H), 6.60-6.52 (m, 4H), 5.09-5.00 (m, 2H), 4.95-4.85 (m, 2H), 4.36-4.28 (m, 1H), 4.24-4.16 (m, 1H), 3.98 (br d, J=2.1 Hz, 1H), 3.52 (br d, J=11.6 Hz, 2H), 3.29-3.24 (m, 5H), 3.05-2.85 (m, 5H), 2.68-2.52 (m, 4H), 2.48-2.44 (m, 2H), 2.39-2.31 (m, 1H), 2.19 (br d, J=6.9 Hz, 2H), 2.00-1.91 (m, 1H), 1.79-1.71 (m, 2H), 1.68-1.57 (m, 1H), 1.21-1.10 (m, 2H).

### Example 97. Preparation of (I-384) (S)-3-(5-(4-((1-(3-fluoro-4-((3R,4R)-7-hydroxy-3-phenylchroman-4-yl)phenyl)piperidin-4-yl)methyl)piperazin-1-yl)-1-oxoisoindolin-2-yl)piperidine-2,6-dione

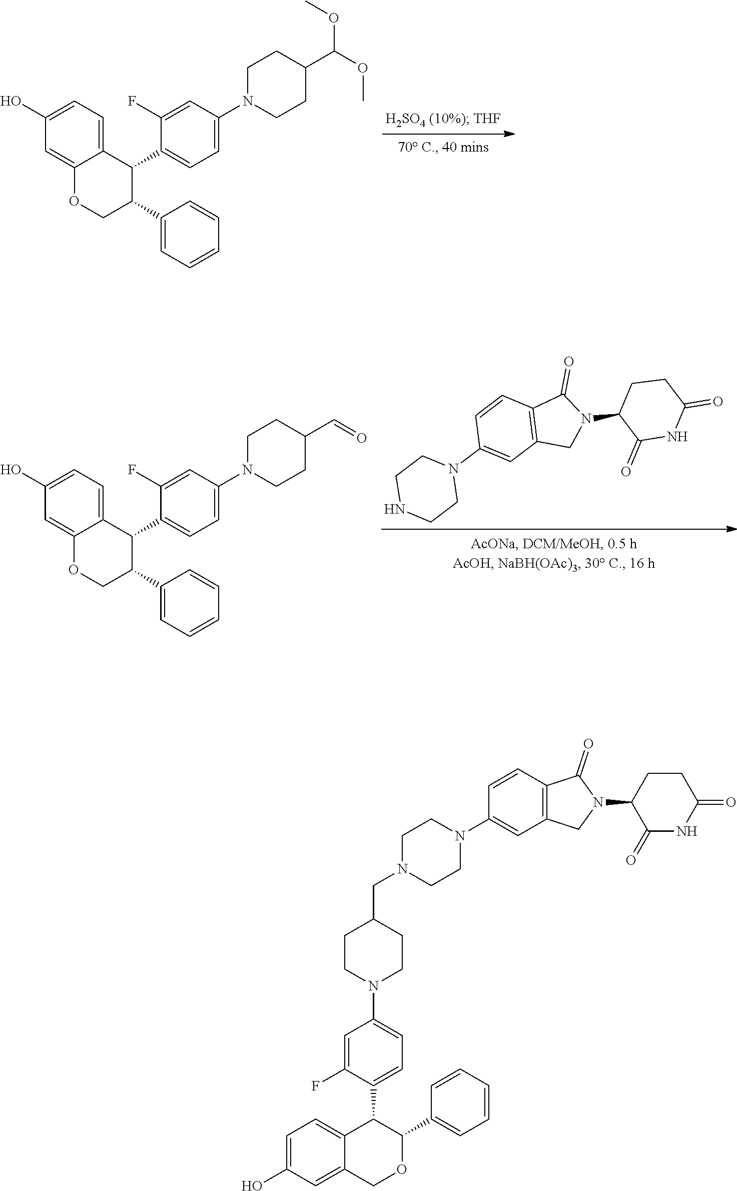 [SMILES: COC(OC)C1CCN(c2ccc([C@H]3c4ccc(O)cc4OC[C@H]3c3ccccc3)c(F)c2)CC1 ; O=CC1CCN(c2ccc([C@H]3c4ccc(O)cc4OC[C@H]3c3ccccc3)c(F)c2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7c7ccccc7)c(F)c6)CC5)CC4)ccc3C2=O)C(=O)N1]

Step 1: To a solution of (3R, 4R)-4-(4-(4-(dimethoxymethyl)piperidin-1-yl)-2-fluorophenyl)-3-phenylisochroman-7-ol (80 mg, 1 Eq, 0.17 mmol) in THF (4 mL) was added 10% H₂SO₄ (4 mL). The mixture was stirred at 70° C. for 40 mins. The reaction mixture was adjusted to pH=7 by adding saturated aqueous NaHCO₃ (30 mL) at 0° C. and diluted with H₂O (20 mL) and extracted with ethyl acetate 50 mL. The organic layer was washed with brine (10 mL). Dried over Na₂SO₄, filtered and concentrated under reduced pressure to give crude product 1-(3-fluoro-4-((3R,4R)-7-hydroxy-3-phenylchroman-4-yl)phenyl)piperidine-4-carbaldehyde (60 mg, 0.14 mmol, 83% yield) as a light red oil, LC-MS (ESI⁺) m/z: 450.1 (M+H₂O)⁺.

Step 2: A mixture of 1-(3-fluoro-4-((3R,4R)-7-hydroxy-3-phenylchroman-4-yl)phenyl)piperidine-4-carbaldehyde (60 mg, 1 Eq, 139 μmol), (S)-3-(1-oxo-5-(piperazin-1-yl)isoindolin-2-yl)piperidine-2,6-dione hydrochloride (51 mg, 1 Eq, 139 μmol), Sodium acetate (34 mg, 3 Eq, 417 μmol) and acetic acid (25 mg, 24 μL, 3 Eq, 417 μmol) in DCM (4 mL) and MeOH (4 mL) was stirred at 30° C. for 0.5 h, then sodium triacetoxyborohydride (59 mg, 2 Eq. 278.1 μmol) was added in the mixture and stirred at 30° C. for 16 h. The mixture was added H₂O (20 mL), then extracted with ethyl acetate (20 mL*2). The combined extracts were dried over anhydrous Na₂SO₄, filtered and concentrated to dryness in vacuum to give a residue. The residue was purified by prep-HPLC: Column: Welch Xtimate C18 150*30 mm+5 um Condition: A: water (FA) B: ACN at the beginning: A (79%) and B (21%) at the end: A: (49%) and B (51%) Gradient Time (min) 7; 100% B Hold Time (min) 3; Flow Rate (ml/min) 25. The aqueous phase was lyophilized to dryness to give (S)-3-(5-(4-((1-(3-fluoro-4-((3R,4R)-7-hydroxy-3-phenylchroman-4-yl)phenyl)piperidin-4-yl)methyl)piperazin-1-yl)-1-oxoisoindolin-2-yl)piperidine-2,6-dione (44.1 mg, 57.5 μmol, 41.4% yield, 97.01% purity) as a white solid. LCMS, HPLC, SFC and NMR confirmed.

LC-MS (ESI⁺) m/z: 744.4 (M+H)⁺.

HPLC: 97.010%, purity at 220 nm.

¹H NMR (400 MHz, DMSO-d₆) δ ppm 10.96 (s, 1H) 9.25-9.44 (m, 1H) 8.16 (s, 0.330H) 7.52 (d, J=8.58 Hz, 1H) 7.04-7.18 (m, 7H) 6.73-6.85 (m, 2H) 6.48-6.62 (m, 3H) 6.29 (dd, J=13.83, 2.15 Hz, 1H) 4.99-5.13 (m, 3H) 4.91 (br d, J=15.26 Hz, 1H) 4.27-4.43 (m, 2H) 4.15-4.25 (m, 1H) 3.52-3.59 (m, 2H) 3.28 (br s, 5H) 2.85-2.96 (m, 1H) 2.51-2.63 (m, 6H) 2.37 (br dd, J=13.17, 4.59 Hz, 1H) 2.18 (br d, J=6.79 Hz, 2H) 1.92-2.00 (m, 1H) 1.60-1.78 (m, 3H) 1.05-1.20 (m, 2H).

### Example 98. Preparation of (I-245) (S)-3-(5-(4-((1-(4-((3R,4R)-3-cyclobutyl-7-hydroxyisochroman-4-yl)phenyl)piperidin-4-yl)methyl)piperazin-1-yl)-1-oxoisoindolin-2-yl)piperidine-2,6-dione

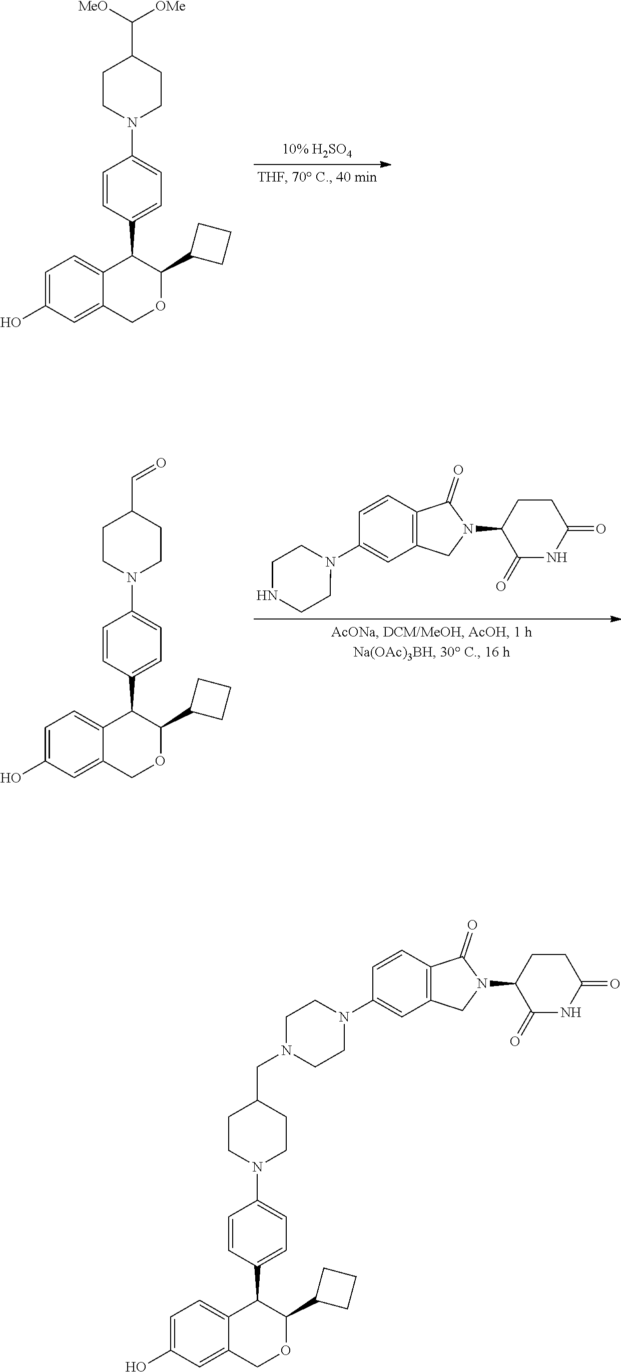 [SMILES: COC(OC)C1CCN(c2ccc([C@@H]3c4ccc(O)cc4CO[C@@H]3C3CCC3)cc2)CC1 ; O=CC1CCN(c2ccc([C@@H]3c4ccc(O)cc4CO[C@@H]3C3CCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@H]7C7CCC7)cc6)CC5)CC4)ccc3C2=O)C(=O)N1]

Step 1: A mixture of (3R,4R)-3-cyclobutyl-4-(4-(4-(dimethoxymethyl)piperidin-1-yl)phenyl)isochroman-7-ol (40 mg, 1 Eq, 91 μmol) was added in THF (3 mL) and 10% H₂SO₄ (3 mL), the mixture was stirred at 70° C. for 40 min. the reaction was quenched with saturated aqueous NaHCO₃ (5 mL), the mixture was treated with H₂O (20 mL) and was extracted with ethyl acetate (50 mL), the organic layer was dried with anhydrous Na₂SO₄, filtered, and concentrated to give 1-(4-((3R,4R)-3-cyclobutyl-7-hydroxyisochroman-4-yl)phenyl)piperidine-4-carbaldehyde (30 mg, 77 μmol, 93%) as a white solid. LC-MS (ESI⁺) m/z: 410.1 (M+H₂O)⁺

Step 2: A mixture of 1-(4-((3R,4R)-3-cyclobutyl-7-hydroxyisochroman-4-yl)phenyl)piperidine-4-carbaldehyde (30 mg, 1 Eq, 77 μmol), (S)-3-(1-oxo-5-(piperazin-1-yl)isoindolin-2-yl)piperidine-2,6-dione hydrochloride (34 mg, 1.2 Eq, 92 μmol, HCl), AcONa (19 mg, 3 Eq, 0.23 mmol) was added in the DCM (2 mL) and MeOH (2 mL), the mixture was stirred at 30° C. for 1 h, then NaBH(OAc); (32 mg. 2 Eq, 0.15 mmol) and acetic acid (13 mg, 13 μL, 3 Eq, 0.23 mmol) was added was stirred at 30° C. for 16 h. the reaction was treated with H₂O (50 mL) and was extracted with ethyl acetate (100 mL), the organic layers were dried with anhydrous Na₂SO₄, filtered, and concentrated to give the crude product. the residue was purified by prep-HPLC (column: Welch Xtimate C18 150*30 mm*5 um; mobile phase: [water (FA)-ACN]; gradient: 16%-46% B over 7 min) to give (S)-3-(5-(4-((1-(4-((3R,4R)-3-cyclobutyl-7-hydroxyisochroman-4-yl)phenyl)piperidin-4-yl)methyl)piperazin-1-yl)-1-oxoisoindolin-2-yl)piperidine-2,6-dione (23 mg, 32 μmol) was obtained as a white solid.

LC-MS (ESI+) m/z: 704.3 (M+H)⁺

HPLC: 99% purity at 220 nm

¹H NMR (400 MHZ, DMSO-d₆) δ=10.94 (s, 1H), 9.20 (br s, 1H), 8.15 (s, 0.397H), 7.52 (d, J=8.6 Hz, 1H), 7.12-7.00 (m, 2H), 6.88 (d, J=8.6 Hz, 2H), 6.76 (d, J=8.6 Hz, 2H), 6.71 (d, J=8.3 Hz, 1H), 6.51 (dd, J=2.4, 8.2 Hz, 1H), 6.46 (d, J=2.0 Hz, 1H), 5.05 (dd, J=5.1, 13.1 Hz, 1H), 4.85 (d, J=15.1 Hz, 1H), 4.68 (d, J=15.1 Hz, 1H), 4.38-4.29 (m, 1H), 4.25-4.14 (m, 1H), 3.69-3.64 (m, 2H), 3.63-3.55 (m, 2H), 2.97-2.84 (m, 1H), 2.64-2.52 (m, 9H), 2.42-2.30 (m, 2H), 2.22 (br d, J=7.0 Hz, 2H), 2.03-1.76 (m, 7H), 1.75-1.65 (m, 4H), 1.28-1.12 (m, 2H).

### Example 99. Preparation of (I-244) (S)-3-(5-(4-((1-(4-((3S,4S)-3-cyclobutyl-7-hydroxyisochroman-4-yl)phenyl)piperidin-4-yl)methyl)piperazin-1-yl)-1-oxoisoindolin-2-yl)piperidine-2,6-dione

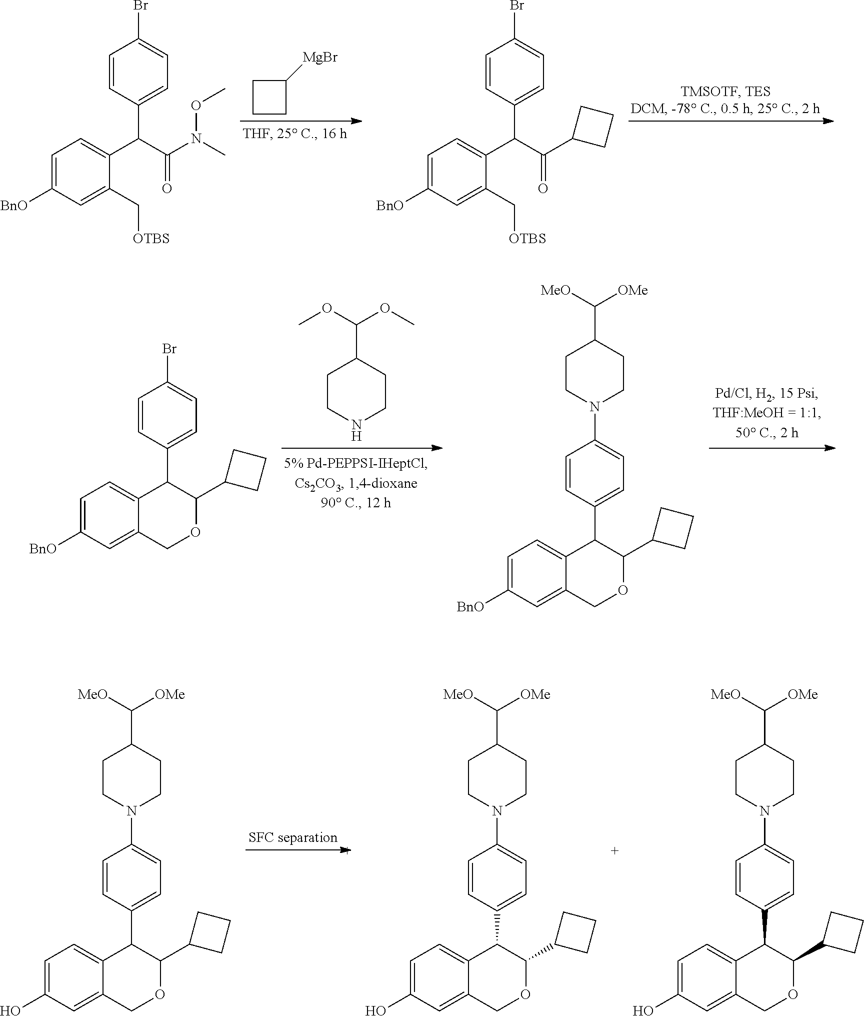

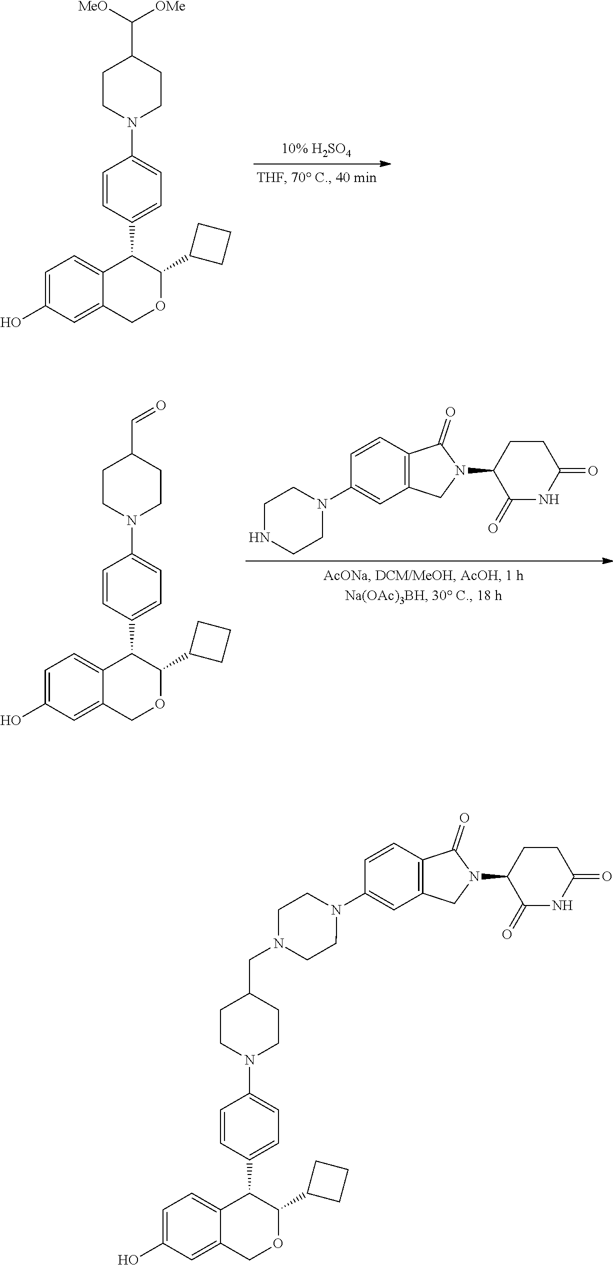 [SMILES: COC(OC)C1CCN(c2ccc([C@H]3c4ccc(O)cc4CO[C@H]3C3CCC3)cc2)CC1 ; O=CC1CCN(c2ccc([C@H]3c4ccc(O)cc4CO[C@H]3C3CCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7C7CCC7)cc6)CC5)CC4)ccc3C2=O)C(=O)N1]

Step 1: A mixture of cyclobutylmagnesium bromide (17 mL, 5 Eq, 8.55 mmol) was added 2-(4-(benzyloxy)-2-(tert-butyldimethylsilyl)oxy)methyl)phenyl)-2-(4-bromophenyl)-N-methoxy-N-methylacetamide (1 g, 1 Eq, 1.71 mmol) in THF (15 mL) at 25° C. under N₂. The suspension was stirred for 16 h at 25° C. the reaction was quenched with saturated aqueous NH₄Cl (50 mL) at 0° C. under N₂, the mixture was treated with H₂O (50 mL) and was extracted with ethyl acetate (100 mL), the combined organic layers were washed with brine (100 mL), dried over anhydrous Na₂SO₄, filtered and concentrated under reduced pressure to give a residue. The residue was purified by flash silica gel chromatography (ISCO®; 20 g SepaFlash® Silica Flash Column, Eluent of 0˜10% ethyl acetate/Petroleum ether gradient @ 35 mL/min) to give compound 2-(4-(benzyloxy)-2-(((tert-butyldimethylsilyl)oxy)methyl)phenyl)-2-(4-bromophenyl)-1-cyclobutylethan-1-one (580 mg, 0.90 mmol). LC-MS (ESI⁺) m/z: 447 (M−132)⁺.

Step 2: A mixture of 2-(4-(benzyloxy)-2-(((tert-butyldimethylsilyl)oxy)methyl)phenyl)-2-(4-bromophenyl)-1-cyclobutylethan-1-one (580 mg, 1 Eq, 1.00 mmol) and triethylsilane (175 mg, 240 μL, 1.5 Eq, 1.5 mmol) in DCM (4 mL) under N₂, the resulting mixture was cooled at −78° C. and trimethylsilyl triflate (334 mg, 277 μL, 1.5 Eq, 1.50 mmol) was added, and the resulting mixture was allowed to stir for 30 min at this temperature, and was stirred at 25° C. for 2 h. the reaction was quenched with saturated aqueous NaHCO₃ (10 mL) at 25° C. under N₂, the mixture was treated with H₂O (50 mL) and was extracted with ethyl acetate (100 mL), the combined organic layers were washed with brine (100 mL), dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜10% ethyl acetate/Petroleum ether gradient @ 30 mL/min) to give compound 7-(benzyloxy)-4-(4-bromophenyl)-3-cyclobutylisochromane (300 mg, 67 μmol).

Step 3: A mixture of 5% Pd-PEPPSI-IHeptCl (86 mg, 0.2 Eq, 89 μmol), Cs₂CO₃ (435 mg, 3 Eq, 1.34 mmol), 4-(dimethoxymethyl)piperidine (106 mg, 112 μL, 1.5 Eq, 668 μmol), 7-(benzyloxy)-4-(4-bromophenyl)-3-cyclobutylisochromane (200 mg, 1 Eq, 445 μmol) was added in 1,4-dioxane (5 mL) and purged with N₂ for 3 times, the mixture was stirred at 90° C. for 12 h under N₂ atmosphere. the mixture was treated with H₂O (50 mL) and was extracted with ethyl acetate (100 mL), the combined organic layers were washed with saturated salt water (100 mL), dried over anhydrous Na₂SO₄, filtered and concentrated under reduced pressure to give a residue. The residue was purified by flash silica gel chromatography (ISCO®; 12 g SepaFlash® Silica Flash Column, Eluent of 0˜10% ethyl acetate/Petroleum ether gradient @ 30 mL/min) to give compound 1-(4-(7-(benzyloxy)-3-cyclobutylisochroman-4-yl)phenyl)-4-(dimethoxymethyl)piperidine (100 mg, 189 μmol). LC-MS (ESI⁺) m/z: 528.3 (M+H)⁺

Step 4: A mixture of 1-(4-(7-(benzyloxy)-3-cyclobutylisochroman-4-yl)phenyl)-4-(dimethoxymethyl)piperidine (100 mg, 1 Eq, 189 μmol), Pd/C (101 mg, 10% Wt, 0.5 Eq, 94.7 μmol) was added in THF (2 mL) and MeOH (2 mL) under N₂ atmosphere, the suspension was degassed and purged with H₂ for 3 times. the mixture was stirred under H₂ (15 Psi) at 50° C. for 2 h. the reaction mixture was filtered and concentrated under reduced pressure to give compound 3-cyclobutyl-4-(4-(4-(dimethoxymethyl)piperidin-1-yl)phenyl)isochroman-7-ol (70 mg, 0.16 mmol). The resident was purified by prep-SFC (column: DAICEL CHIRALCEL OD (250 mm*30 mm, 10 um); mobile phase: CO₂-EtOH (0.1% NH₃H₂O); B %: 40% %, isocratic elution mode) to give (3S,4S)-3-cyclobutyl-4-(4-(4-(dimethoxymethyl)piperidin-1-yl)phenyl)isochroman-7-ol (30 mg, 69 μmol) and (3R,4R)-3-cyclobutyl-4-(4-(4-(dimethoxymethyl)piperidin-1-yl)phenyl)isochroman-7-ol (40 mg, 91 μmol) was obtained as a white solid.

5 of LC-MS (ESI⁺) m/z: 438.4 (M+H)⁺

5A of LC-MS (ESI⁺) m/z: 438.3 (M+H)⁺

Step 5: A mixture of (3S,4S)-3-cyclobutyl-4-(4-(4-(dimethoxymethyl)piperidin-1-yl)phenyl)isochroman-7-ol (30 mg, 1 Eq, 69 μmol) was added in THF (3 mL) and 10% H₂SO₄ (3 mL), the mixture was stirred at 70° C. for 40 min. the reaction was quenched with saturated aqueous NaHCO₃ (5 mL), the mixture was treated with H₂O (20 mL) and was extracted with ethyl acetate (50 mL), the organic layer was dried with anhydrous Na₂SO₄, filtered, and concentrated to give 1-(4-((3S,4S)-3-cyclobutyl-7-hydroxyisochroman-4-yl)phenyl)piperidine-4-carbaldehyde (25 mg, 64 μmol, 93%) as a white solid. LC-MS (ESI⁺) m/z: 410.1 (M+H₂O)⁺

Step 6: A mixture of 1-(4-((3S,4S)-3-cyclobutyl-7-hydroxyisochroman-4-yl)phenyl)piperidine-4-carbaldehyde (25 mg, 1 Eq, 64 μmol), (S)-3-(1-oxo-5-(piperazin-1-yl)isoindolin-2-yl)piperidine-2,6-dione hydrochloride (28 mg, 1.2 Eq, 77 μmol, HCl), AcONa (16 mg, 3 Eq, 0.19 mmol) was added in the DCM (2 mL) and MeOH (2 mL), the mixture was stirred at 30° C. for 1 h, then NaBH(OAc)₃ (27 mg, 2 Eq, 0.13 mmol) and acetic acid (12 mg, 11 μL, 3 Eq, 0.19 mmol) was added was stirred at 30° C. for 16 h. the reaction was treated with H₂O (50 mL) and was extracted with ethyl acetate (100 mL), the organic layers were dried with anhydrous Na₂SO₄, filtered, and concentrated to give the crude product. the residue was purified by prep-HPLC (column: Welch Xtimate C18 150*30 mm*5 um; mobile phase: [water (FA)-ACN]; gradient: 16%-46% B over 7 min) to give (S)-3-(5-(4-((1-(4-((3S,4S)-3-cyclobutyl-7-hydroxyisochroman-4-yl)phenyl)piperidin-4-yl)methyl)piperazin-1-yl)-1-oxoisoindolin-2-yl)piperidine-2,6-dione (21 mg, 30.3 μmol) was obtained as a white solid.

LC-MS (ESI⁺) m/z: 704.2 (M+H)⁺

HPLC: 99% purity at 220 nm

¹H NMR (400 MHZ, DMSO-d₆) ppm=10.94 (s, 1H), 9.35-9.07 (m, 1H), 8.15 (s, 0.451H), 7.52 (d, J=8.6 Hz, 1H), 7.10-7.01 (m, 2H), 6.88 (d. J=8.6 Hz, 2H), 6.76 (d, J=8.7 Hz, 2H), 6.71 (d, J=8.3 Hz, 1H), 6.51 (dd, J=2.2, 8.4 Hz, 1H), 6.46 (d, J=2.0 Hz, 1H), 5.05 (dd, J=5.0, 13.2 Hz, 1H), 4.85 (d, J=15.4 Hz, 1H), 4.68 (d, J=15.0 Hz, 1H), 4.39-4.28 (m, 1H), 4.26-4.12 (m, 1H), 3.70-3.64 (m, 2H), 3.59 (br d, J=8.8 Hz, 2H), 2.99-2.81 (m, 1H), 2.65-2.52 (m, 9H), 2.42-2.29 (m, 2H), 2.22 (br d, J=7.2 Hz, 2H), 2.01-1.76 (m, 7H), 1.75-1.61 (m, 5H), 1.31-1.13 (m, 2H).

### Example 100. Preparation of (I-246) (S)-3-(5-(4-((1-(4-((3R,4R)-3-cyclopentyl-7-hydroxyisochroman-4-yl)phenyl)piperidin-4-yl)methyl)piperazin-1-yl)-1-oxoisoindolin-2-yl)piperidine-2,6-dione

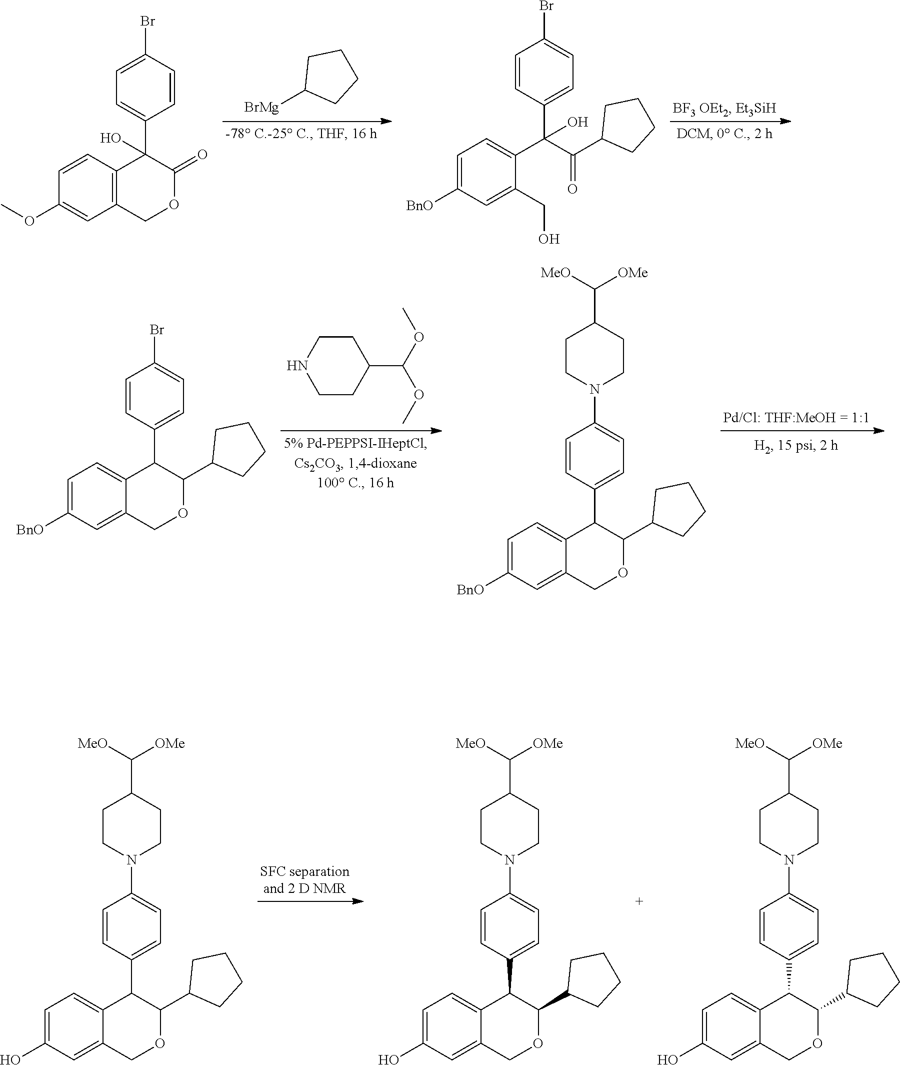

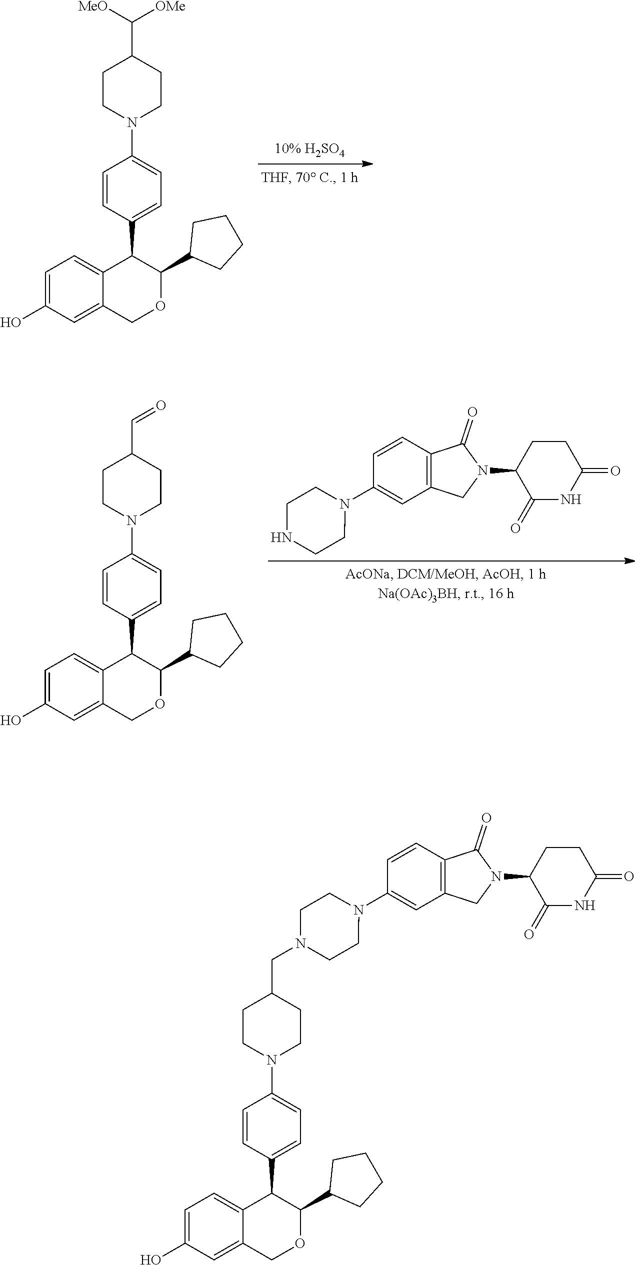 [SMILES: COC(OC)C1CCN(c2ccc([C@@H]3c4ccc(O)cc4CO[C@@H]3C3CCCC3)cc2)CC1 ; O=CC1CCN(c2ccc([C@@H]3c4ccc(O)cc4CO[C@@H]3C3CCCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@H]7C7CCCC7)cc6)CC5)CC4)ccc3C2=O)C(=O)N1]

Step 1: A mixture of 7-(benzyloxy)-4-(4-bromophenyl)-4-hydroxyisochroman-3-one (1 g, 1 Eq, 2 mmol) in THF (20 mL) was stirred at −78° C. with N₂, then cyclopentylmagnesium bromide (2 g, 9 mL, 1 molar, 4 Eq, 9 mmol) was added to the mixture and stirred at −78° C. for 2 hours. The reaction was warmed to 25° C. slowly and stirred for 14 hours. The reaction was quenched with saturated aqueous NH₄Cl solution (20 mL) and was extracted with ethyl acetate (40 mL*2). The combined organic layers were dried over anhydrous Na₂SO₄, filtered, and concentrated to dryness in vacuum to give a residue. The residue was subjected to column chromatography over silica gel (petroleum ether/ethyl acetate from 100/0 to 70/30). The pure fractions were collected and concentrated to dryness in vacuum to give 2-(4-(benzyloxy)-2-(hydroxymethyl)phenyl)-2-(4-bromophenyl)-1-cyclopentyl-2-hydroxyethan-1-one (500 mg, 90.716% purity, 40% yield) as a yellow solid. LC-MS (ESI⁺) m/z: 477.0 (M-OH)⁺.

Step 2: A mixture of 2-(4-(benzyloxy)-2-(hydroxymethyl)phenyl)-2-(4-bromophenyl)-1-cyclopentyl-2-hydroxyethan-1-one (410 mg, 1 Eq, 828 μmol) in DCM (8 mL) was bubbled with N₂ for 3 min and was stirred at 0° C., then triethylsilane (481 mg, 659 μL, 5 Eq, 4.14 mmol) and boron trifluoride etherate (235 mg, 209 μL, 2 Eq. 1.66 mmol) was added to the mixture at 0° C. The reaction was stirred at 0° C. for 2 hour. The reaction was quenched with saturated aqueous NaHCO₃ (20 mL) at ice bath and extracted with DCM (20 mL*2). The combined organic layers were dried over anhydrous Na₂SO₄, filtered and concentrated to dryness in vacuum to give a residue. The residue was subjected to column chromatography over silica gel (gradient elution: 0-20% ethyl acetate in petroleum ether). The pure fractions were collected and concentrated to dryness in vacuo to give 7-(benzyloxy)-4-(4-bromophenyl)-3-cyclopentylisochromane (390 mg, 84.073% purity, 85.5% yield) as a colorless oil.

Step 3: A mixture of Pd-PEPPSI-IHeptCI (164 mg, 0.2 Eq, 168 μmol), Cs₂CO₃ (823 mg, 3 Eq, 2.52 mmol), 4-(dimethoxymethyl)piperidine (201 mg, 211 μL, 1.5 Eq, 1.26 mmol) and 7-(benzyloxy)-4-(4-bromophenyl)-3-cyclopentylisochromane (390 mg, 1 Eq, 842 μmol) in 1,4-Dioxane (16 mL) was heated to 100° C. for 16 hour under N₂ atmosphere. The mixture was diluted with H₂O (20 mL) and extracted with ethyl acetate (30 mL*2). The combined organic layers were dried over anhydrous Na₂SO₄, filtered and concentrated to dryness in vacuum to give a residue. The residue was subjected to column chromatography over silica gel (gradient elution: 0-30% ethyl acetate in petroleum ether). The pure fractions were collected and concentrated to dryness in vacuum to give 1-(4-(7-(benzyloxy)-3-cyclopentylisochroman-4-yl)phenyl)-4-(dimethoxymethyl)piperidine (336 mg, 100% purity, 73.7% yield) as a light yellow solid. LC-MS (ESI⁺) m/z: 542.0 (M+H)⁺.

Step 4: A mixture of 1-(4-(7-(benzyloxy)-3-cyclopentylisochroman-4-yl)phenyl)-4-(dimethoxymethyl)piperidine (330 mg, 1 Eq, 609 μmol), wet Pd/C (10% wt, 64.8 mg, 1 Eq, 609 μmol) in THF (10 mL) and MeOH (10 mL) was degassed and purged with H₂ for 3 times, then the mixture was stirred at 15 psi for 2 hour under H₂ atmosphere. The reaction was filtered and concentrated to dryness in vacuum to give 3-cyclopentyl-4-(4-(4-(dimethoxymethyl)piperidin-1-yl)phenyl)isochroman-7-ol (250 mg, 100% purity, 90.9% yield) as a yellow solid. LC-MS (ESI⁺) m/z: 452.3 (M+H)⁺.

Step 5: A mixture of 3-cyclopentyl-4-(4-(4-(dimethoxymethyl)piperidin-1-yl)phenyl)isochroman-7-ol was purified by SFC: (column: DAICEL CHIRALPAK IE 250 mm*30 mm, 10 um); Condition: CO₂-i-PrOH (0.1% NH₃H₂O); B %: 45%-45%; Flow Rate: 80 ml/min; peak 1:2.048 min; peak 2:2.294 min) to give (3R,4R)-3-cyclopentyl-4-(4-(4-(dimethoxymethyl)piperidin-1-yl)phenyl)isochroman-7-ol (115 mg, 98.715% purity) and (3S,4S)-3-cyclopentyl-4-(4-(4-(dimethoxymethyl)piperidin-1-yl)phenyl)isochroman-7-ol (130 mg, 98.505% purity) as yellow foam.

Step 6: A solution of (3R,4R)-3-cyclopentyl-4-(4-(4-(dimethoxymethyl)piperidin-1-yl)phenyl)isochroman-7-ol (115 mg, 1 Eq, 255 μmol) in THF (1.5 mL) and 10% H₂SO₄ (3.0 mL) was stirred at 70° C. for 1 hour. The reaction mixture was poured into saturated aqueous NaHCO₃ (20 mL) solution, then was extracted with ethyl acetate (20 mL*3). The combined organic layers were washed with brine (30 mL), dried over anhydrous Na₂SO₄, filtered and concentrated under reduced pressure to give 1-(4-((3R,4R)-3-cyclopentyl-7-hydroxyisochroman-4-yl)phenyl)piperidine-4-carbaldehyde (100 mg, crude) as a yellow oil. LC-MS (ESI⁺) m/z: 424.1 (M+H₂O)⁺.

Step 7: A solution of (3R,4R)-3-cyclopentyl-4-(4-(4-(dimethoxymethyl)piperidin-1-yl)phenyl)isochroman-7-ol (100 mg, 1 Eq, 221 μmol), (S)-3-(1-oxo-5-(piperazin-1-yl)isoindolin-2-yl)piperidine-2,6-dione hydrochloride (89 mg, 1.1 Eq, 244 μmol) and sodium acetate (91 mg, 5 Eq, 1.11 mmol) in DCM (3 mL) and MeOH (3 mL) was stirred at 25° C. for 1 b, then acetic acid (27 mg, 25 μL, 2 Eq, 443 μmol) and sodium triacetoxybydroborate (94 mg, 2 Eq, 443 μmol) was added. The mixture was stirred at 25° C. for 16 hour. The mixture was diluted with H₂O (20 ml) and was extracted with dichloromethane (20 ml*3). The combined organic layers were dried over anhydrous Na₂SO₄, filtered and concentrated under reduced pressure to give a colorless oil (150 mg, crude). The residue was purified by prep-HPLC (column: Welch Xtimate C18 150*30 mm; mobile phase: [water (FA)-ACN]; B % 18%-48%, 7 min) to give (S)-3-(5-(4-((1-(4-((3R,4R)-3-cyclopentyl-7-hydroxyisochroman-4-yl)phenyl)piperidin-4-yl)methyl)piperazin-1-yl)-1-oxoisoindolin-2-yl)piperidine-2,6-dione (110.5 mg, 72% yield, 97.443% purity) was obtained as a pink solid. LC-MS (ESI⁺) m/z: 718.8 (M+H)⁺.

LCMS: calc. for C43H₅₁N₅O₅: 717.39, found: [M+H]⁺ 718.8.

HPLC: 97.443% purity at 220 nm.

¹H NMR (400 MHZ, DMSO-d₆) δ=10.95 (s, 1H), 9.32-9.09 (m, 1H), 8.18 (s, 0.192H), 7.52 (d, J=8.7 Hz, 1H), 7.09-6.98 (m, 4H), 6.76 (br d, J=8.7 Hz, 2H), 6.72 (d, J=8.5 Hz, 1H), 6.49 (dd, J=2.3, 8.3 Hz, 1H), 6.44 (d, J=2.0 Hz, 1H), 5.05 (dd, J=5.1, 13.2 Hz, 1H), 4.86 (d, J=15.4 Hz, 1H), 4.68 (br d, J=15.3 Hz, 1H), 4.37-4.16 (m, 2H), 3.68 (br d, J=1.5 Hz, 1H), 3.62-3.56 (m, 2H), 3.41-3.32 (m, 9H), 2.94-2.85 (m, 1H), 2.61-2.54 (m, 3H), 2.40-2.31 (m, 1H), 2.21 (br d, J=7.0 Hz, 2H), 1.99-1.91 (m, 1H), 1.79 (br d, J=11.4 Hz, 2H), 1.62 (br d, J=4.5 Hz, 3H), 1.55-1.47 (m, 2H), 1.44-1.30 (m, 4H), 1.27-1.14 (m, 3H).

### Example 101. Preparation of (I-247) (S)-3-(5-(4-((1-(4-((3S,4S)-3-cyclopentyl-7-hydroxyisochroman-4-yl)phenyl)piperidin-4-yl)methyl)piperazin-1-yl)-1-oxoisoindolin-2-yl)piperidine-2,6-dione

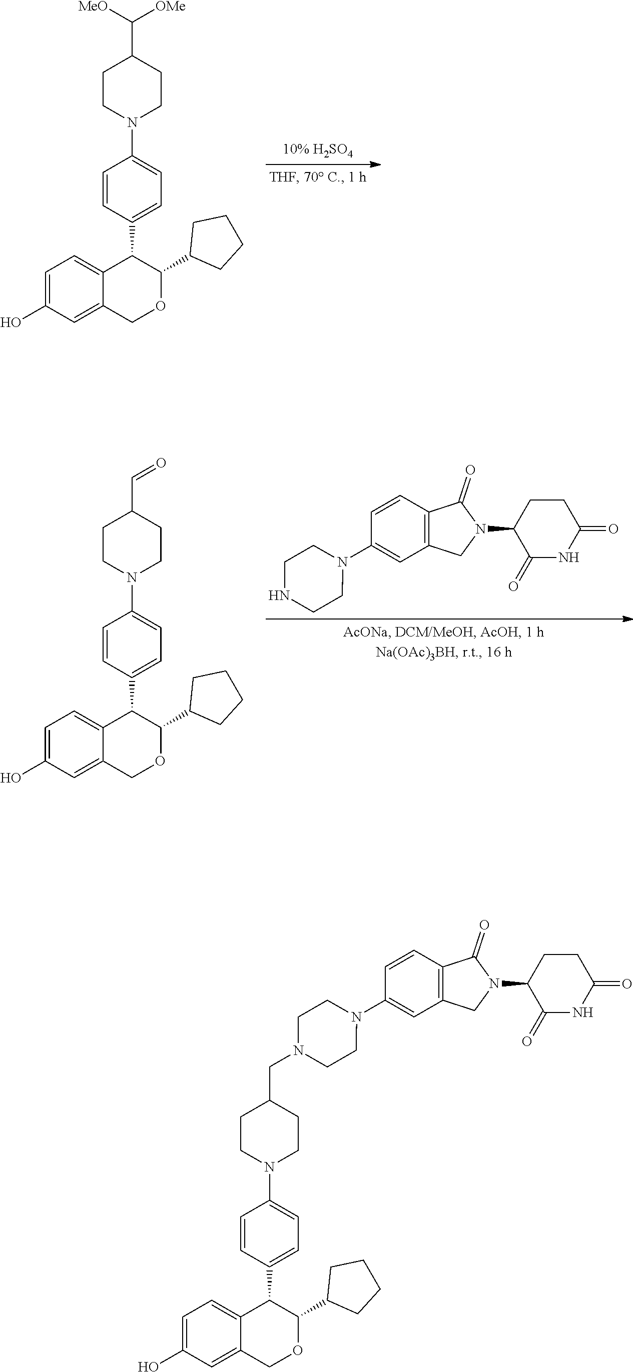 [SMILES: COC(OC)C1CCN(c2ccc([C@H]3c4ccc(O)cc4CO[C@H]3C3CCCC3)cc2)CC1 ; O=CC1CCN(c2ccc([C@H]3c4ccc(O)cc4CO[C@H]3C3CCCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7C7CCCC7)cc6)CC5)CC4)ccc3C2=O)C(=O)N1]

Step 1: A solution of (3S,4S)-3-cyclopentyl-4-(4-(4-(dimethoxymethyl)piperidin-1-yl)phenyl)isochroman-7-ol (130 mg, 1 Eq, 288 μmol) in THF (1.5 mL) and 10% H₂SO₄ (3 mL) was stirred at 70° C. for 1 hour. The reaction mixture was poured into 10 mL saturated aqueous NaHCO₃ solution, then was extracted with ethyl acetate (20 mL*3). The combined organic layers were washed with brine (30 mL), dried over anhydrous Na₂SO₄, filtered and concentrated under reduced pressure to give 1-(4-((3S,4S)-3-cyclopentyl-7-hydroxyisochroman-4-yl)phenyl)piperidine-4-carbaldehyde (110 mg, crude) as a yellow oil. LC-MS (ESI⁺) m/z: 424.1 (M+H₂O)⁺.

Step 2: A solution of 1-(4-((3S,4S)-3-cyclopentyl-7-hydroxyisochroman-4-yl)phenyl)piperidine-4-carbaldehyde (110 mg, 1 Eq, 271 μmol), (S)-3-(1-oxo-5-(piperazin-1-yl)isoindolin-2-yl)piperidine-2,6-dione (109 mg, 1.1 Eq, 298 μmol, HCl salt) and sodium acetate (111 mg, 5 Eq, 1.36 mmol) in DCM (3 mL) and MeOH (3 mL) was stirred at 25° C. for 1 h, then acetic acid (33 mg, 31 μL, 2 Eq, 542 μmol) and sodium triacetoxyhydroborate (115 mg, 2 Eq, 542 μmol) was added. The mixture was stirred at 25° C. for 16 hour. The mixture was diluted with H₂O (20 mL) and was extracted with dichloromethane (20 ml*3). The combined organic layers were dried over anhydrous Na₂SO₄, filtered and concentrated under reduced pressure to give a colorless oil (180 mg, crude). The residue was purified by prep-HPLC (column: Welch Xtimate C18 150*30 mm; mobile phase: [water (FA)-ACN]; B % 18%-48%, 7 min) to give (S)-3-(5-(4-((1-(4-((3S,4S)-3-cyclopentyl-7-hydroxyisochroman-4-yl)phenyl)piperidin-4-yl)methyl)piperazin-1-yl)-1-oxoisoindolin-2-yl)piperidine-2,6-dione (102.6 mg, 56% yield, 98.936% purity) was obtained as a pink solid. LC-MS (ESI⁺) m/z: 718.4 (M+H)⁺.

LCMS: calc. for C₄₃H₅₁N₅O₅:717.39, found: [M′H]⁺ 718.4.

HPLC: 98.936% purity at 220 nm.

¹H NMR (400 MHZ, DMSO-d₆) δ=10.96 (s, 1H), 9.30-9.12 (m, 1H), 8.19 (s, 0.167H), 7.52 (d, J=8.7 Hz, 1H), 7.09-7.00 (m, 4H), 6.77 (d, J=8.7 Hz, 2H), 6.73 (d, J=8.5 Hz, 1H), 6.50 (dd, J=2.4, 8.2 Hz, 1H), 6.44 (d, J=2.3 Hz, 1H), 5.09-5.02 (m, 1H), 4.87 (d, J=15.4 Hz, 1H), 4.69 (d, J=15.1 Hz, 1H), 4.37-4.30 (m, 1H), 4.24-4.17 (m, 1H), 3.69 (d, J=2.1 Hz, 1H), 3.64-3.55 (m, 2H), 3.36 (br d, J=5.7 Hz, 9H), 2.96-2.85 (m, 1H), 2.63-2.54 (m, 3H), 2.42-2.33 (m, 1H), 2.22 (br d, J=7.2 Hz, 2H), 2.01-1.92 (m, 1H), 1.80 (br d, J=12.0 Hz, 2H), 1.63 (br d, J=4.5 Hz, 3H), 1.52 (br d. J=4.5 Hz, 2H), 1.46-1.31 (m, 4H), 1.27-1.15 (m, 3H).

### Example 102. Preparation of (I-383) (S)-3-(5-(4-((1-(3-fluoro-4-((3S,4S)-7-hydroxy-3-phenylchroman-4-yl)phenyl)piperidin-4-yl)methyl)piperazin-1-yl)-1-oxoisoindolin-2-yl)piperidine-2,6-dione

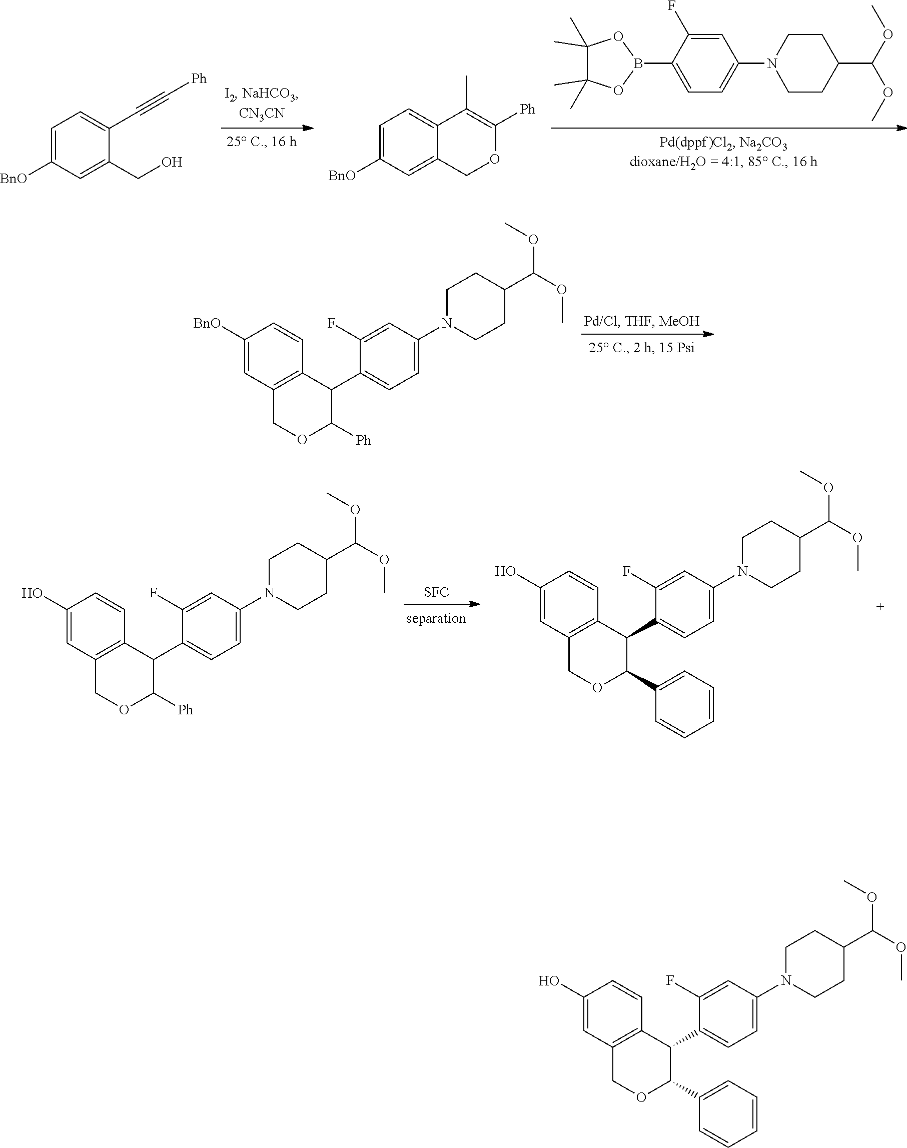

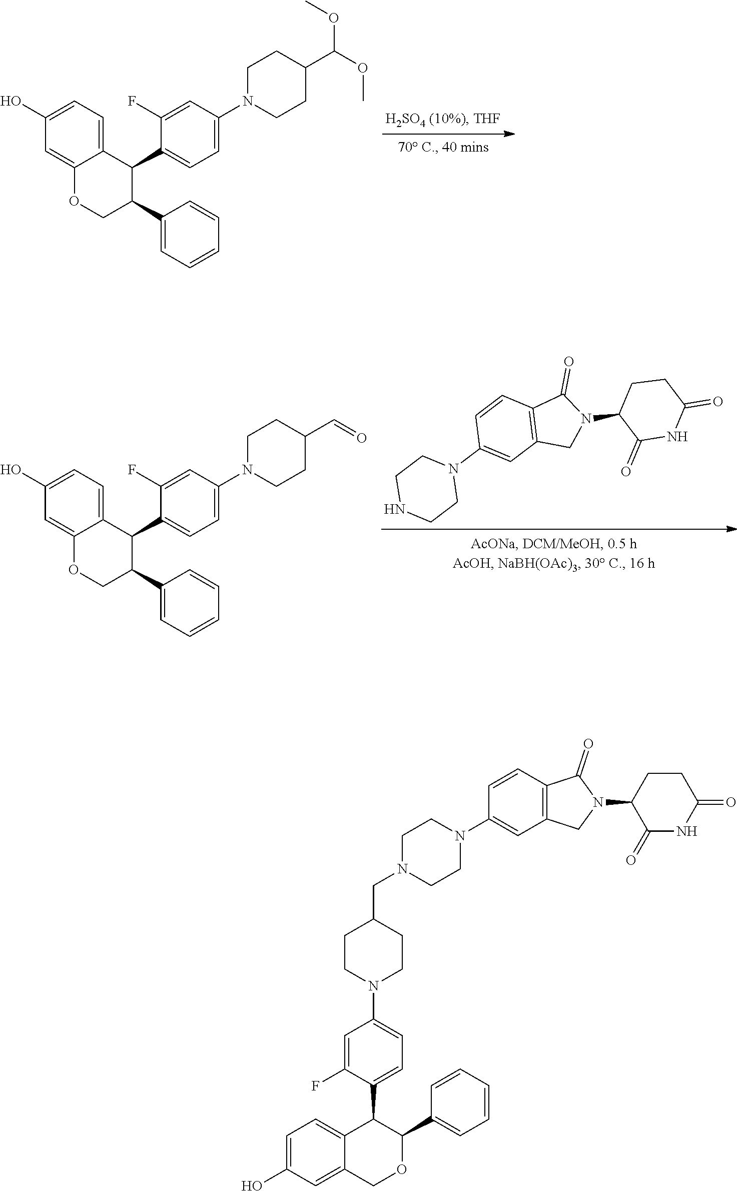 [SMILES: COC(OC)C1CCN(c2ccc([C@@H]3c4ccc(O)cc4OC[C@@H]3c3ccccc3)c(F)c2)CC1 ; O=CC1CCN(c2ccc([C@@H]3c4ccc(O)cc4OC[C@@H]3c3ccccc3)c(F)c2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccccc7)c(F)c6)CC5)CC4)ccc3C2=O)C(=O)N1]

Step 1: To a solution of (5-(benzyloxy)-2-(phenylethynyl)phenyl) methanol (400 mg, 1 Eq, 1.3 mmol) in MeCN (20 mL) was added sodium bicarbonate (321 mg, 3 Eq, 3.8 mmol) and iodine (969 mg, 3 Eq, 3.8 mmol). The mixture was stirred at 25° C. for 16 h. TLC (petroleum ether:ethyl acetate=10:1, R_{f}=0.5) showed one new spot was observed. The reaction mixture was quenched by saturated aqueous Na₂SO₃ (20 mL) and diluted with H₂O (20 mL) and extracted with ethyl acetate (60 mL). The organic layer was dried over anhydrous Na₂SO₄, filtered and concentrated under vacuum to give a yellow oil. The yellow oil was purified by silica gel column chromatography (ISCO®; 20 g SepaFlash® Silica Flash Column, Eluent of 0˜20% ethyl acetate/petroleum ether gradient @ 24 mL/min) to give 7-(benzyloxy)-4-iodo-3-phenyl-1H-isochromene (220 mg, 480 μmol, 37.7% yield, 96.092% purity) as a black solid.

Step 2: To a mixture of 4-(dimethoxymethyl)-1-(3-fluoro-4-(4,4,5,5-tetramethyl-1,3,2-dioxaborolan-2-yl)phenyl)piperidine (208 mg, 1.1 Eq, 550 μmol), sodium carbonate (132 mg, 2.5 Eq. 1.3 mmol) and 4-(dimethoxymethyl)-1-(3-fluoro-4-(4,4,5,5-tetramethyl-1,3,2-dioxaborolan-2-yl)phenyl)piperidine (208 mg, 1.1 Eq, 550 μmol) in 1,4-dioxane (8 mL) and H₂O (2 mL) was added Pd(dppf) Cl₂ (37 mg, 0.1 Eq, 50 μmol) and stirred at 85° C. under N₂ atmosphere for 16 h. TLC (petroleum ether:ethyl acetate=3:1, R_{f}=0.6) showed one new spot was observed. The reaction mixture was diluted with H₂O (30 mL) and extracted with ethyl acetate 90 mL. The organic layer was washed with brine (20 mL), dried over anhydrous Na₂SO₄, filtered and concentrated under reduced pressure to give a residue. The residue was purified by flash silica gel chromatography (ISCO®; 20 g SepaFlash® Silica Flash Column, Eluent of 0˜30% ethyl acetate/petroleum ether gradient @ 24 mL/min) and the organic layer was concentrated in vacuum to give 1-(4-(7-(benzyloxy)-3-phenyl-1H-isochromen-4-yl)-3-fluorophenyl)-4-(dimethoxymethyl)piperidine (190 mg, 303 μmol, 60.7% yield, 90.242% purity) as a light red oil. LC-MS (ESI⁺) m/z: 566.2 (M+H)⁺.

Step 3: A mixture of 1-(4-(7-(benzyloxy)-3-phenyl-1H-isochromen-4-yl)-3-fluorophenyl)-4-(dimethoxymethyl)piperidine (190 mg, 1 Eq, 336 μmol), Pd/C (190 mg, 10% purity, 0.5 Eq, 179 μmol) in THF (6 mL) and MeOH (6 mL) was degassed and purged with H₂ for 3 times, and then the mixture was stirred at 25° C. for 2 h under H₂ atmosphere (15 psi). The reaction was filtered and concentrated to dryness in vacuum to give 4-(4-(4-(dimethoxymethyl)piperidin-1-yl)-2-fluorophenyl)-3-phenylisochroman-7-ol (190 mg, 398 μmol, 91% purity) as a light red oil. LC-MS (ESI⁺) m/z; 478.1 (M+H)⁺.

Step 4: The light red oil was purified by SFC Column: DAICEL CHIRALPAK

AD (250 mm*30 mm, 10 um) Condition: A: CO₂ B: ETOH (0.1% NH₃H₂O) at the beginning: A (45%) and B (55%) at the end: A: (45%) and B (55%) Gradient Time (min) 45; 100% B Hold Time (min); Flow Rate (ml/min) 80. The fractions were filtered and concentrated under reduced pressure to give (3S,4S)-4-(4-(4-(dimethoxymethyl)piperidin-1-yl)-2-fluorophenyl)-3-phenylisochroman-7-ol (100 mg, 209 μmol, 52.6% yield, 100% purity) and (3R,4R)-4-(4-(4-(dimethoxymethyl)piperidin-1-yl)-2-fluorophenyl)-3-phenylisochroman-7-ol (80 mg, 0.17 mmol, 42% yield, 100% purity) as a light red oil.

LC-MS (ESI⁺) m/z: 478.1 (M+H)⁺.

Step 5: To a solution of (3S,4S)-4-(4-(4-(dimethoxymethyl)piperidin-1-yl)-2-fluorophenyl)-3-phenylisochroman-7-ol (100 mg, 1 Eq, 209 μmol) in THF (4 mL) was added 10% H₂SO₄ (4 mL). The mixture was stirred at 70° C. for 40 mins. The reaction mixture was adjusted to pH=7 by adding saturated aqueous NaHCO₃ (30 mL) at 0° C. and diluted with H₂O (20 mL) and extracted with ethyl acetate 50 mL. The organic layer was washed with brine (10 mL). Dried over anhydrous Na₂SO₄, filtered and concentrated under reduced pressure to give crude product 1-(3-fluoro-4-((3S,4S)-7-hydroxy-3-phenylchroman-4-yl)phenyl)piperidine-4-carbaldehyde (80 mg, 0.19 mmol, 89% yield) as a light red oil LC-MS (ESI⁺) m/z: 450.1 (M+H₂O)⁺.

Step 6: A mixture of 1-(3-fluoro-4-((3S,4S)-7-hydroxy-3-phenylchroman-4-yl)phenyl)piperidine-4-carbaldehyde (80 mg, 1 Eq, 185.4 μmol), (S)-3-(1-oxo-5-(piperazin-1-yl)isoindolin-2-yl)piperidine-2,6-dione (68 mg, 1 Eq, 185.4 μmol, HCl salt), sodium acetate (46 mg, 3 Eq, 557 μmol) and acetic acid (34 mg, 32 μL, 3 Eq, 557 μmol) in DCM (4 mL) and MeOH (4 mL) was stirred at 30° C. for 0.5 h, then sodium triacetoxyborohydride (79 mg, 2 Eq, 371 μmol) was added in the mixture and stirred at 30° C. for 16 h. TLC (DCM/MeOH=10/1, R_{f}=0.6) showed a new spot was detected. The mixture was added H₂O (20 mL), then extracted with ethyl acetate (20 mL*2). The combined extracts were dried over anhydrous Na₂SO₄, filtered and concentrated to dryness in vacuum to give a residue. The residue was purified by prep-HPLC: Column: Welch Xtimate C18 150*30 mm*5 um Condition: A: water (FA) B: ACN at the beginning: A (79%) and B (21%) at the end: A: (49%) and B (51%) Gradient Time (min) 7; 100% B Hold Time (min) 3.2; Flow Rate (ml/min) 25. The aqueous phase was lyophilized to dryness to give (S)-3-(5-(4-((1-(3-fluoro-4-((3S,4S)-7-hydroxy-3-phenylchroman-4-yl)phenyl)piperidin-4-yl)methyl)piperazin-1-yl)-1-oxoisoindolin-2-yl)piperidine-2,6-dione (46.1 mg, 61.8 μmol, 33.3% yield, 99.694% purity) as a white solid. LCMS, HPLC, SFC and NMR confirmed.

LC-MS (ESI⁺) m/z: 744.4 (M+H)⁺

HPLC: 99.694%, purity at 220 nm.

¹H NMR (400 MHZ, DMSO-d₆) δ ppm 10.96 (s, 1H) 9.28-9.41 (m, 1H) 8.16 (s, 0.375H) 7.52 (d, J=8.82 Hz, 1H) 7.02-7.17 (m, 7H) 6.74-6.85 (m, 2H) 6.49-6.61 (m, 3H) 6.29 (dd, J=13.83, 2.27 Hz, 1H) 5.00-5.12 (m, 3H) 4.91 (br d, J=15.38 Hz, 1H) 4.29-4.42 (m, 2H) 4.16-4.25 (m, 1H) 3.50-3.61 (m, 2H) 3.28 (br s, 5H) 2.84-2.97 (m, 1H) 2.51-2.62 (m, 6H) 2.33-2.41 (m, 1H) 2.14-2.22 (m, 2H) 1.92-2.01 (m, 1H) 1.60-1.79 (m, 3H) 1.05-1.19 (m, 2H).

### Example 103. Preparation of (I-230) (S)-3-(5-(4-((1-(4-((3R,4R)-3-(cyclopropylmethyl)-7-hydroxyisochroman-4-yl)phenyl)piperidin-4-yl)methyl)piperazin-1-yl)-1-oxoisoindolin-2-yl)piperidine-2,6-dione

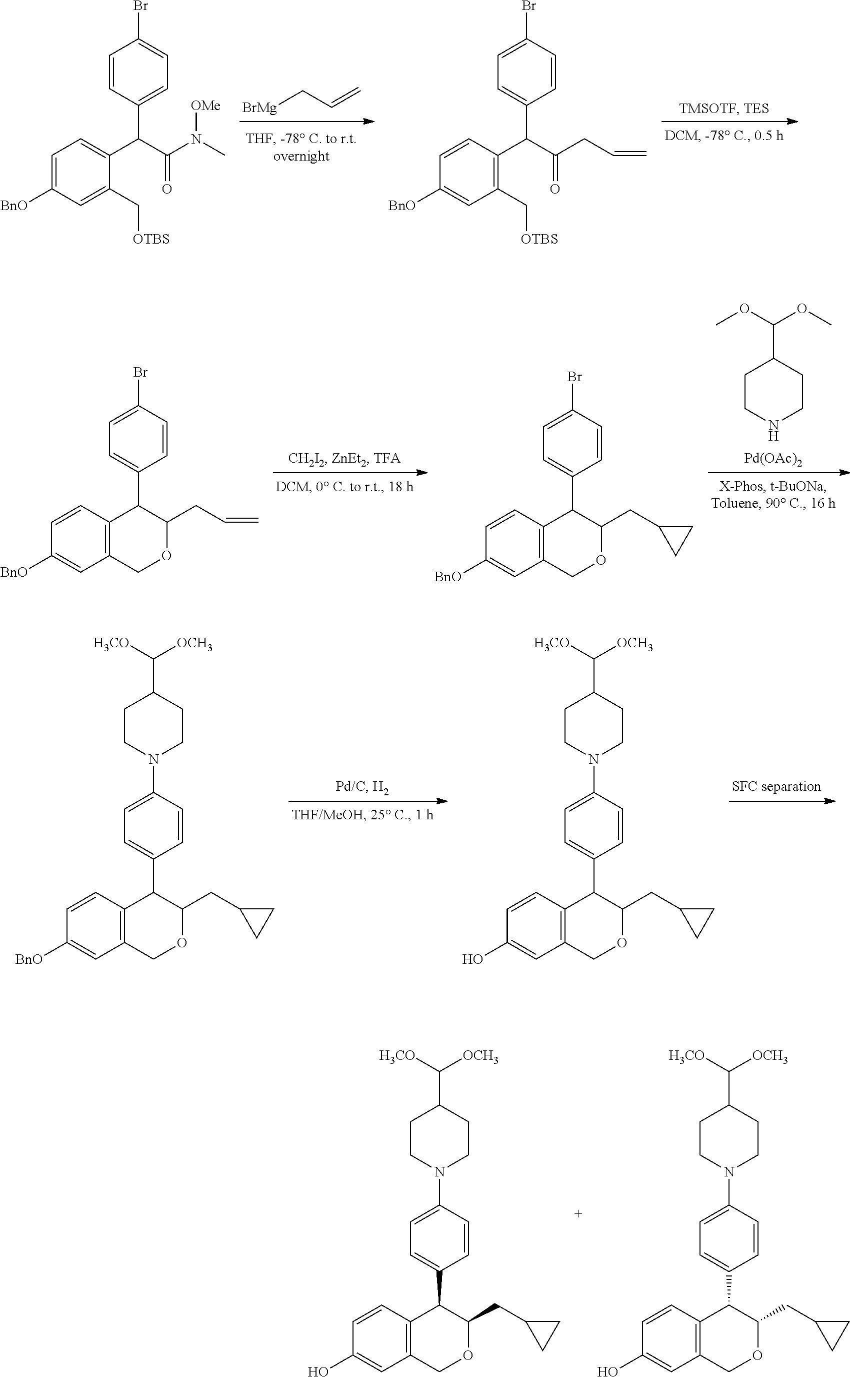

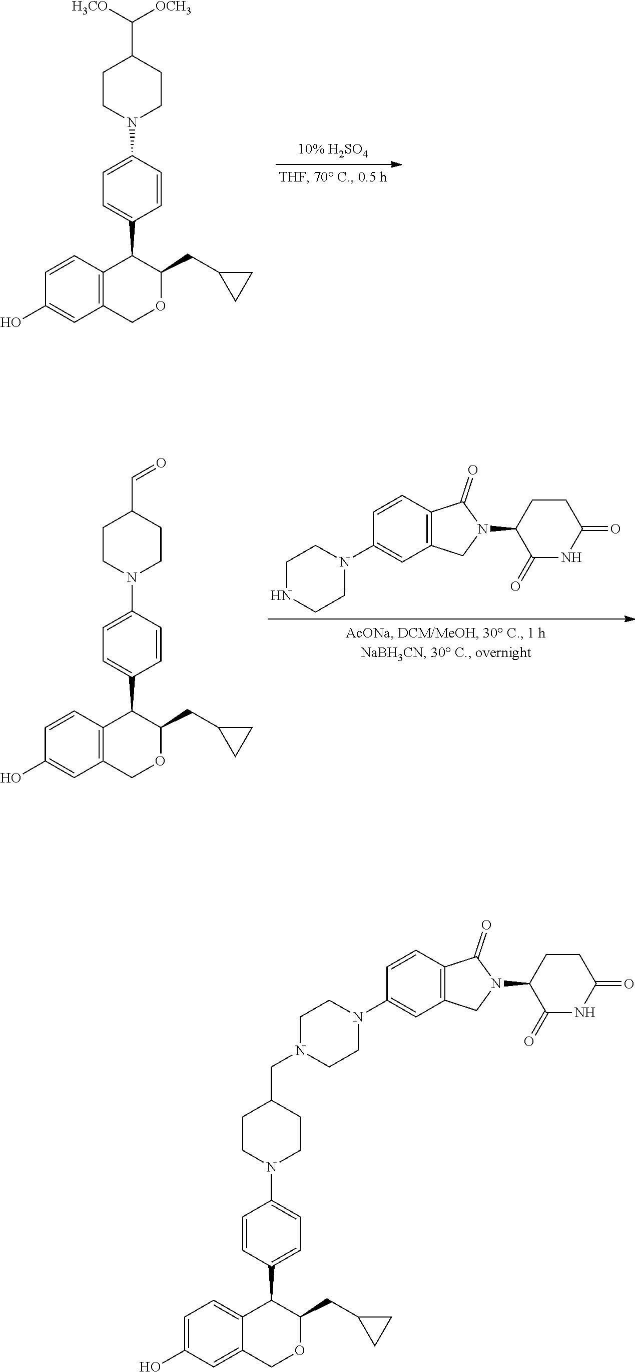

Step 1: Allylmagnesium bromide (0.75 g, 5.1 mL, 1 molar, 3 Eq, 5.1 mmol) was added in THF (4 mL) with N₂ atmosphere and stirred at −78° C., then 2-(4-(benzyloxy)-2-(((tert-butyldimethylsilyl)oxy) methyl)phenyl)-2-(4-bromophenyl)-N-methoxy-N-methylacetamide (1.0 g, 1 Eq, 1.7 mmol) in THF (1 mL) was added to the mixture and stirred at −78° C.-r.t. for 16 h with N₂ atmosphere. The reaction was quenched with aq. NH₄Cl (10 mL) at ice bath, then the mixture was added H₂O (100 mL) and extracted with ethyl acetate (100 mL×2). The combined extracts were dried over anhydrous Na₂SO₄, filtered and concentrated to dryness in vacuo to give a residue. The residue was subjected to column chromatography over silica gel (petroleum ether/ethyl acetate from 100/0 to 90/10). The pure fractions were collected and concentrated to dryness in vacuo to give 1-(4-(benzyloxy)-2-(((tert-butyldimethylsilyl)oxy)methyl)phenyl)-1-(4-bromophenyl) pent-4-en-2-one (680 mg, 60% yield, 85.9% purity) as a colorless oil. LC-MS (ESI⁺) m/z: 434.7 (M−132)⁺.

Step 2: A mixture of 1-(4-(benzyloxy)-2-(((tert-butyldimethylsilyl)oxy)methyl)phenyl)-1-(4-bromophenyl) pent-4-en-2-one (680 mg, 1 Eq, 1.03 mmol) and triethylsilane (180 mg, 248 μL, 1.5 Eq, 1.55 mmol) in DCM (6 mL) was added dropwise trimethylsilyl triflate (345 mg, 286 μL, 1.5 Eq, 1.55 mmol) at −78° C. with N₂. Then the reaction was stirred at −78° C. for 0.5 h with N₂. The reaction was quenched with aq. NaHCO₃ (5 mL) at ice bath, then the mixture was added H₂O (100 mL) and extracted with ethyl acetate (100 mL×2). The combined extracts were dried over anhydrous Na₂SO₄, filtered and concentrated to dryness in vacuo to give a residue. The residue was purified by HPLC (column: Welch Xtimate C18 150*30 mm*5 um; mobile phase: [water (FA)-ACN]; B % 75%-100%, 9 min). The aqueous phase was collected to dryness to give 3-allyl-7-(benzyloxy)-4-(4-bromophenyl)isochromane (210 mg, 45.0% yield, 96.4% purity) as a yellow oil. LC-MS (ESI⁺) m/z: 436.7 (M+H)⁺.

Step 3: A mixture of trifluoroacetic acid (75.8 mg, 50.6 μL, 2 Eq, 665 μmol) in DCM (4 mL) was stirred at 0° C. with N₂, then diethylzinc (123 mg, 997 μL, 1 molar, 3 Eq, 997 μmol) was added to the mixture and stirred at 0° C. for 30 min. Then methylene iodide (267 mg, 80.3 μL, 3 Eq, 997 μmol) in DCM (1 mL) was added to the mixture and stirred at 0° C. for 30 min. Then 3-allyl-7-(benzyloxy)-4-(4-bromophenyl)isochromane (150 mg, 1 Eq, 332 μmol) in DCM (1 mL) was added to reaction at 0° C. and stirred at 0° C.-n.t for 15 b. The reaction was quenched with aq. NaHCO₃ (10 mL) at ice bath. The mixture was added H₂O (100 mL) and extracted with ethyl acetate (100 mL×2). The combined organic layers were dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 7-(benzyloxy)-4-(4-bromophenyl)-3-(cyclopropylmethyl)isochromane (150 mg, 92.0% yield, 91.6% purity) as a yellow solid. LC-MS (ESI⁺) m/z: 450.8 (M+H)⁺.

Step 4: A mixture of 7-(benzyloxy)-4-(4-bromophenyl)-3-(cyclopropylmethyl)isochromane (130 mg, 1 Eq, 265 μmol), 2-(Dicyclohexylphosphanyl)-2′,4′,6′-tris(isopropyl) biphenyl (25.3 mg, 0.2 Eq, 53.0 μmol), palladium diacetate (8.93 mg, 0.15 Eq, 39.8 μmol), 4-(dimethoxymethyl)piperidine (63.3 mg, 1.5 Eq, 398 μmol) and sodium 2-methylpropan-2-olate (38.2 mg, 1.5 Eq, 398 μmol) in toluene (5 mL) was stirred at r.t. N₂ was bubbled into the mixture for 5 min. Then the reaction was heated to 90° C. for 16 h. The mixture was added H₂O (50 mL) and extracted with ethyl acetate (50 mL×2). The combined organic layers were dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 1-(4-((3R,4R)-7-(benzyloxy)-3-(cyclopropylmethyl)isochroman-4-yl)phenyl)-4-(dimethoxymethyl)piperidine (95 mg, 64% yield, 94.8% purity) as a white solid. LC-MS (ESI⁺) m/z: 528.3 (M+H)⁺.

Step 5: A mixture of 1-(4-(7-(benzyloxy)-3-(cyclopropylmethyl)isochroman-4-yl)phenyl)-4-(dimethoxymethyl)piperidine (95 mg, 1 Eq, 0.17 mmol), Pd—C (100 mg, 10% wt 5.5 Eq, 940 μmol) and dihydrogen (0.34 mg, 1 Eq, 0.17 mmol) in MeOH (3 mL) and THF (3 mL) was degassed and purged with H₂ for 3 times, then the mixture was stirred at 25° C. for 1 h with H₂ atmosphere (15 psi). The mixture was filtered and concentrated to dryness in vacuo to give a white solid. The white solid was purified by SFC (column: DAICEL CHIRALPAK AD (250 mm*30 mm, 10 um); mobile phase: [CO₂-EtOH (0.1% NH₃H₂O)]; B % 40%-40%, 45 min). The aqueous phase was collected to dryness in vacuo to give (3R,4R)-3-(cyclopropylmethyl)-4-(4-(4-(dimethoxymethyl)piperidin-1-yl)phenyl)isochroman-7-ol (25 mg, 33% yield, 97.7% purity) and (3S,4S)-3-(cyclopropylmethyl)-4-(4-(4-(dimethoxymethyl)piperidin-1-yl)phenyl)isochroman-7-ol (25 mg, 33% yield, 100% purity) as white solids. LC-MS (ESI⁺) m/z: 438.4 (M+H)⁺.

Step 6: A mixture of (3R,4R)-3-(cyclopropylmethyl)-4-(4-(4-(dimethoxymethyl)piperidin-1-yl)phenyl)isochroman-7-ol (25 mg, 1 Eq, 56 μmol) in THF (1 mL) and 10% H₂SO₄ (1 mL) was stirred at 70° C. for 0.5 h. The reaction was adjust to pH=8 with aq. NaHCO₃ at 0° C. Then the mixture was added H₂O (20 mL) and extracted with ethyl acetate (20 mL×2). The combined organic layers were dried over anhydrous Na₂SO₄, filtered and concentrated to dryness in vacuo to give 1-(4-((3R,4R)-3-(cyclopropylmethyl)-7-hydroxyisochroman-4-yl)phenyl)piperidine-4-carbaldehyde (22 mg, 98% yield, 97.1% purity) as a yellow solid. LC-MS (ESI⁺) m/z: 392.1 (M+H)⁺.

Step 7: A mixture of 1-(4-((3R,4R)-3-(cyclopropylmethyl)-7-hydroxyisochroman-4-yl)phenyl)piperidine-4-carbaldehyde (22 mg, 1 Eq, 55 μmol), (S)-3-(1-oxo-5-(piperazin-1-yl)isoindolin-2-yl)piperidine-2,6-dione, HCl salt (24 mg, 1.2 Eq, 65 μmol), sodium acetate (22 mg, 5 Eq, 0.27 mmol) and acetic acid (9.8 mg, 9.4 μL, 3 Eq. 0.16 mmol) in DCM (3 mL) and MeOH (3 mL) was stirred at 30° C. for 1 h, then sodium triacetoxyborohydride (23 mg, 2 Eq, 0.11 mmol) was added to the mixture and stirred at 30° C. for 16 h. Then the mixture was added H₂O (50 mL) and extracted with ethyl acetate (50 mL×2). The combined extracts were dried over anhydrous Na₂SO₄, filtered and concentrated to dryness in vacuo to give a residue. The residue was purified by prep-HPLC (column: Welch Xtimate C18 150*30 mm*5 um; mobile phase: [water (FA)-ACN]; B % 10%-50%, 8 min) to give (S)-3-(5-(4-((1-(4-((3R,4R)-3-(cyclopropylmethyl)-7-hydroxyisochroman-4-yl)phenyl)piperidin-4-yl)methyl)piperazin-1-yl)-1-oxoisoindolin-2-yl)piperidine-2,6-dione (25.1 mg, 65% yield, 99.4% purity) as a white solid. LC-MS (ESI⁺) m/z: 704.2 (M+H)⁺.

LCMS: calc. for C₄₂H₄₉N₅O₅:703.37, found: [M+H]⁺ 704.2.

HPLC: 99.426% purity at 220 nm.

¹HNMR (400 MHz, DMSO-d₆) δ=10.98 (s, 1H), 9.39-9.14 (m, 1H), 8.22 (s, 1H), 7.53 (d, J=8.6 Hz, 1H), 7.12-7.04 (m, 2H), 6.99 (d, J=8.6 Hz, 2H), 6.83-6.72 (m, 3H), 6.56-6.45 (m, 2H), 5.07 (dd, J=5.1, 13.4 Hz, 1H), 4.86 (d, J=15.4 Hz, 1H), 4.72 (br d, J=15.4 Hz, 1H), 4.39-4.30 (m, 1H), 4.27-4.17 (m, 1H), 3.81 (br t, J=8.0 Hz, 1H), 3.69 (br s, 1H), 3.61 (br d, J=10.8 Hz, 2H), 3.30 (br s, 5H), 2.99-2.85 (m, 1H), 2.65-2.55 (m, 4H), 2.51-2.48 (m, 2H), 2.45-2.30 (m, 1H), 2.23 (br d, J=7.0 Hz, 2H), 2.03-1.92 (m, 1H), 1.87-1.76 (m, 2H), 1.68 (br s, 1H), 1.29-1.15 (m, 2H), 1.10-0.97 (m, 2H), 0.85-0.74 (m, 1H), 0.45-0.32 (m, 2H), 0.06-−0.05 (m, 2H).

### Example 104. Preparation of (I-231) (S)-3-(5-(4-((1-(4-((3S,4S)-3-(cyclopropylmethyl)-7-hydroxyisochroman-4-yl)phenyl)piperidin-4-yl)methyl)piperazin-1-yl)-1-oxoisoindolin-2-yl)piperidine-2,6-dione

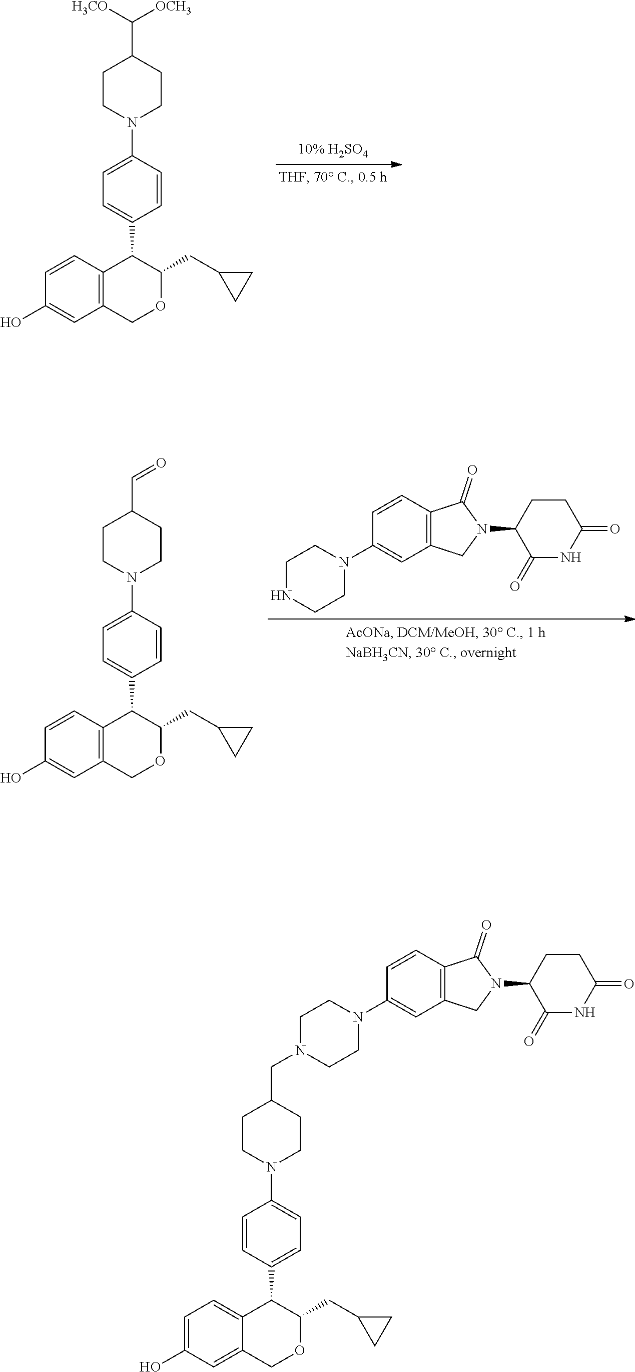 [SMILES: COC(OC)C1CCN(c2ccc([C@H]3c4ccc(O)cc4CO[C@H]3CC3CC3)cc2)CC1 ; O=CC1CCN(c2ccc([C@H]3c4ccc(O)cc4CO[C@H]3CC3CC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7CC7CC7)cc6)CC5)CC4)ccc3C2=O)C(=O)N1]

Step 1: A mixture of (3S,4S)-3-(cyclopropylmethyl)-4-(4-(4-(dimethoxymethyl)piperidin-1-yl)phenyl)isochroman-7-ol (20 mg, 1 Eq, 46 μmol) in THF (1 mL) and 10% H₂SO₄ (1 mL) was stirred at 70° C. for 0.5 h. The reaction was adjust to pH=8 with aq. NaHCO₃ (5 mL) at 0° C. Then the mixture was added H₂O (20 mL) and extracted with ethyl acetate (20 mL×2). The combined organic layers were dried over anhydrous Na₂SO₄, filtered and concentrated to dryness in vacuo to give 1-(4-((3S,4S)-3-(cyclopropylmethyl)-7-hydroxyisochroman-4-yl)phenyl)piperidine-4-carbaldehyde (18 mg, 96% yield, 95.5% purity) as a yellow solid. LC-MS (ESI⁺) m/z: 392.1 (M+H)⁺.

Step 2: A mixture of 1-(4-((3S,4S)-3-(cyclopropylmethyl)-7-hydroxyisochroman-4-yl)phenyl)piperidine-4-carbaldehyde (18 mg, 1 Eq, 44 μmol), (S)-3-(1-oxo-5-(piperazin-1-yl)isoindolin-2-yl)piperidine-2,6-dione, HCl salt (19 mg, 1.2 Eq, 53 μmol), sodium acetate (18 mg, 5 Eq, 0.22 mmol) and acetic acid (7.9 mg, 7.6 μL, 3 Eq, 0.13 mmol) in DCM (3 mL) and MeOH (3 mL) was stirred at 30° C. for 1 h, then sodium triacetoxyborohydride (19 mg, 13 μL, 2 Eq, 88 μmol) was added to the mixture and was stirred at 30° C. for 16 h. Then the mixture was added H₂O (20 mL) and extracted with ethyl acetate (20 mL×2). The combined extracts were dried over anhydrous Na₂SO₄, filtered and concentrated to dryness in vacuo to give a residue. The residue was purified by prep-HPLC (column: Welch Xtimate C18 150*30 mm*5 um; mobile phase: [water (FA)-ACN]; B % 10%-50%, 8 min) to give (S)-3-(5-(4-((1-(4-((3S,4S)-3-(cyclopropylmethyl)-7-hydroxyisochroman-4-yl)phenyl)piperidin-4-yl)methyl)piperazin-1-yl)-1-oxoisoindolin-2-yl)piperidine-2,6-dione (19.4 mg, 25.9 μmol, 59% yield, 93.9% purity) as a white solid. LC-MS (ESI⁺) m/z: 704.4 (M+H)⁺.

LCMS: calc. for C₄₂H₄₉N₅O₅:703.37, found: [M+H]⁺704.4.

HPLC: 93.932% purity at 220 nm.

¹HNMR (400 MHz, DMSO-d₆) δ=10.98 (s, 1H), 9.26 (br s, 1H), 8.22 (s, 1H), 7.53 (d, J=8.5 Hz, 1H), 7.10-7.04 (m, 2H), 6.99 (d, J=8.7 Hz, 2H), 6.81-6.72 (m, 3H), 6.52 (dd, J=2.4, 8.3 Hz, 1H), 6.47 (d, J=2.3 Hz, 1H), 5.07 (dd, J=5.0, 13.2 Hz, 1H), 4.90-4.83 (m, 1H), 4.72 (br d, J=15.1 Hz, 1H), 4.38-4.31 (m, 1H), 4.25-4.18 (m, 1H), 3.81 (br t, J=8.0 Hz, 1H), 3.69 (br s, 1H), 3.61 (br d, J=10.1 Hz, 2H), 3.30 (br s, 6H), 2.98-2.85 (m, 1H), 2.65-2.54 (m, 4H), 2.50-2.48 (m, 1H), 2.38 (dq, J=4.2, 13.2 Hz, 1H), 2.23 (br d, J=7.0 Hz, 2H), 2.02-1.93 (m, 1H), 1.86-1.76 (m, 2H), 1.68 (br s, 1H), 1.29-1.14 (m, 2H), 1.10-0.97 (m, 2H), 0.86-0.73 (m, 1H), 0.45-0.31 (m, 2H), 0.07-−0.07 (m, 2H).

### Example 105. Preparation of (I-391) 1-(4-(2-((1-(4-((3S,4R)-7-hydroxy-3-phenylisochroman-4-yl)phenyl)piperidin-4-yl)methyl)-2,8-diazaspiro[4.5]decan-8-yl)phenyl)dihydropyrimidine-2,4 (1H,3H)-dione

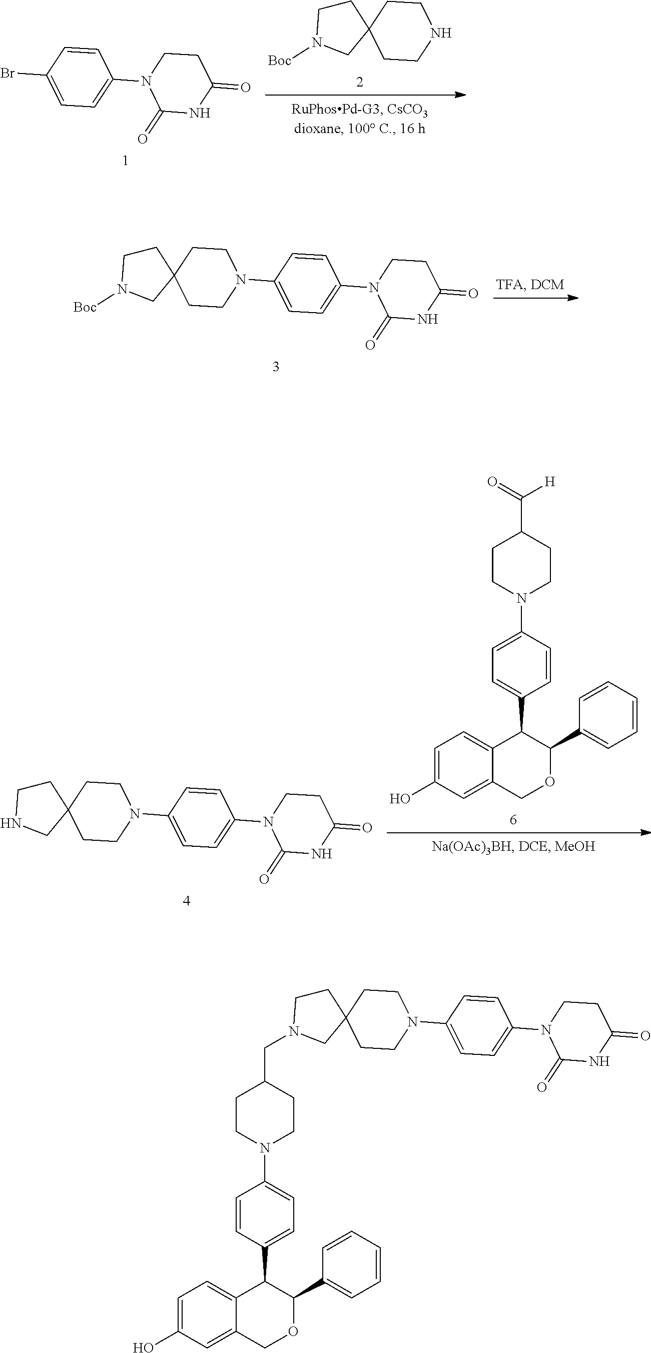

Step 1: A mixture of compound 1 (893 mg, 3.72 mmol), Cs₂CO₃ (1.21 g, 3.72 mmol) and RuPhos-Pd-G3 (155 mg, 186 μmol) in 1,4-Dioxane (10 mL) was degassed and purged with N₂ for 3 times, and then the mixture was stirred at 100° C. for 16 h under N₂ atmosphere. The reaction was quenched with water (20 mL) and extracted with EtOAc (20 mL×3). The combined organic layers were washed with brine (50 mL), dried over Na₂SO₄, filtered and concentrated under reduced pressure to give a residue. The residue was purified by flash silica gel chromatography (ethyl acetate in petroleum ether=0% to 100%) to give the product of compound 3 (200 mg, 15% yield) as a white solid. LC-MS (ESI⁺) m/z: 429.1 (M+H)⁺.

Step 2: To a solution of compound 3 (200 mg, 467 μmol) in DCM (3 mL) was added TFA (1 g, 1 mL, 0.01 mol) at 20° C. The mixture was stirred at 20° C. for 2 h. The solvent was removed to yield a residue which was purified by preparative HPLC (column: Boston Green ODS 150*30 mm*5 um; mobile phase: [water (HCl)-ACN]; B %: 0%-20%, 11 min), followed by lyophilization to give the product of compound 4 (130 mg, 84.8% yield) as a brown solid. LC-MS (ESI⁺) m/z: 329.3 (M+H)⁺.

Step 3: To a solution of compound 4 (45 mg, 0.11 mmol) in DCE (10 mL) and MeOH (1 mL) was added compound 5 (36 mg, 0.11 mmol) and stirred at 20° C. for 10 min. Then NaBH(OAc)₃ (69 mg, 0.33 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 52%-82% B over 11 min), followed by lyophilization to give 1-(4-(2-((1-(4-((3S,4R)-7-hydroxy-3-phenylisochroman-4-yl)phenyl)piperidin-4-yl)methyl)-2,8-diazaspiro[4.5]decan-8-yl)phenyl)dihydropyrimidine-2,4 (1H,3H)-dione (25 mg, 32% yield) as a yellow solid. LC-MS (ESI⁺) m/z: 726.3 (M+H)⁺.

LCMS: calc. for C₄₅H₅₁N₅O₄:725.93, found: [M+H]⁺726.3.

HPLC: 100.0% purity at 254 nm.

¹HNMR (500 MHz, DMSO-d₆): δ 10.24 (s, 1H), 9.27 (s, 1H), 7.17-7.10 (m, 4H), 7.10-7.04 (m, 3H), 6.91 (d, J=9.0 Hz, 2H), 6.78 (d, J=8.1 Hz, 1H), 6.63-6.58 (m, 2H), 6.57-6.52 (m, 4H), 5.07 (d, J=15.4 Hz, 1H), 4.99 (d, J=2.9 Hz, 1H), 4.90 (d, J=15.3 Hz, 1H), 4.02 (d, J=2.7 Hz, 1H), 3.68 (t, J=6.7 Hz, 2H), 3.54-3.43 (m, 2H), 3.19-3.01 (m, 4H), 2.67 (t, J=6.6 Hz, 2H), 2.52 (d, J=1.8 Hz, 2H), 2.47-2.41 (m, 2H), 2.32 (s, 2H), 2.21 (d, J=7.2 Hz, 2H), 1.73 (d, J=12.1 Hz, 2H), 1.66-1.53 (m, 6H), 1.49 (dt, J=3.6, 7.1 Hz, 1H), 1.12 (q, J=11.9 Hz, 2H).

### Example 106. Preparation of (I-396) 1-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)dihydropyrimidine-2,4 (1H,3H)-dione

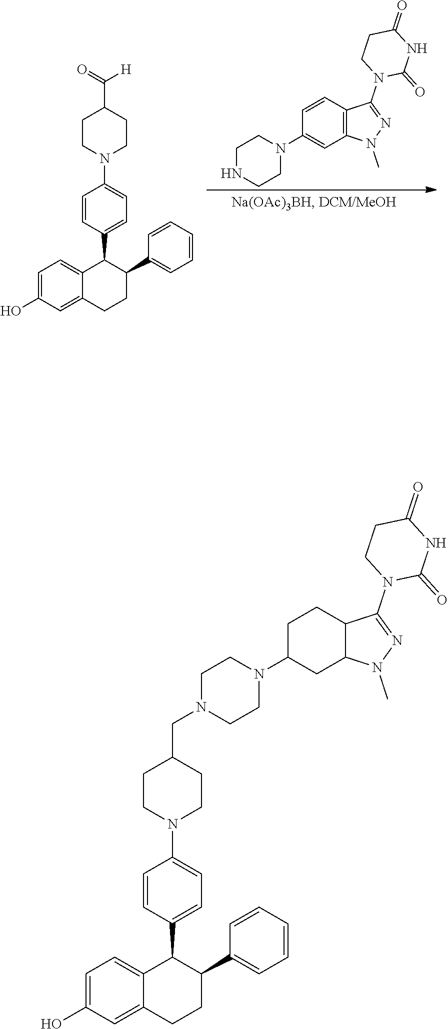 [SMILES: O=CC1CCN(c2ccc([C@@H]3c4ccc(O)cc4CC[C@@H]3c3ccccc3)cc2)CC1 ; Cn1nc(N2CCC(=O)NC2=O)c2ccc(N3CCNCC3)cc21 ; CN1N=C(N2CCC(=O)NC2=O)C2CCC(N3CCN(CC4CCN(c5ccc([C@@H]6c7ccc(O)cc7CC[C@@H]6c6ccccc6)cc5)CC4)CC3)CC21]

Step 1: To a solution of 1-(4-((3S,4R)-7-hydroxy-3-phenylisochroman-4-yl)phenyl)piperidine-4-carbaldehyde (40 mg, 1 eq, 97 μmol) in DCE (10 mL) and MeOH (1 mL) was added 1-(1-methyl-6-(piperazin-1-yl)-1H-indazol-3-yl)dihydropyrimidine-2,4 (1H,3H)-dione (32 mg, 1 eq, 97 μmol) and stirred at 20° C. for 10 min. Then sodium triacetoxyhydroborate (62 mg, 3 eq, 0.29 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 48%-78% B over 11 min), followed by lyophilization give 1-(6-(4-((1-(4-((3S,4R)-7-hydroxy-3-phenylisochroman-4-yl)phenyl)piperidin-4-yl)methyl)piperazin-1-yl)-1-methyl-1H-indazol-3-yl)dihydropyrimidine-2,4 (1H,3H)-dione (32 mg, 46% yield) as a white solid. LC-MS (ESI⁺) m/z: 726.4 (M+H)⁺.

LCMS: calc. for C43H₄₇N₇O₄: 725.89, found: [M+H]⁺726.4.

HPLC: 100.0% purity at 254 nm.

¹HNMR (400 MHz, DMSO-d₆): δ 10.50 (s, 1H), 9.28 (s, 1H), 7.44 (d, J=9.1 Hz, 1H), 7.18-7.03 (m, 5H), 6.90 (d, J=9.2 Hz, 1H), 6.84-6.74 (m, 2H), 6.64-6.59 (m, 2H), 6.55 (d, J=6.4 Hz, 4H), 5.07 (d, J=15.4 Hz, 1H), 5.00 (d, J=3.0 Hz, 1H), 4.90 (d, J=15.3 Hz, 1H), 4.03 (d, J=2.7 Hz, 1H), 3.94-3.80 (m, 5H), 3.50 (d, J=12.4 Hz, 2H), 3.21 (s, 6H), 2.73 (t, J=6.6 Hz, 2H), 2.55 (s, 2H), 2.46 (d, J=3.5 Hz, 2H), 2.19 (d, J=7.0 Hz, 2H), 1.75 (d, J=12.0 Hz, 2H), 1.63 (s, 1H), 1.25-1.06 (m, 2H).

### Example 107. Preparation of (I-392) 1-(4-(2-((1-(4-((3S,4R)-7-hydroxy-3-phenylisochroman-4-yl)phenyl)piperidin-4-yl)methyl)-2,8-diazaspiro[4.5]decan-8-yl)-2-methylphenyl)dihydropyrimidine-2,4 (1H,3H)-dione

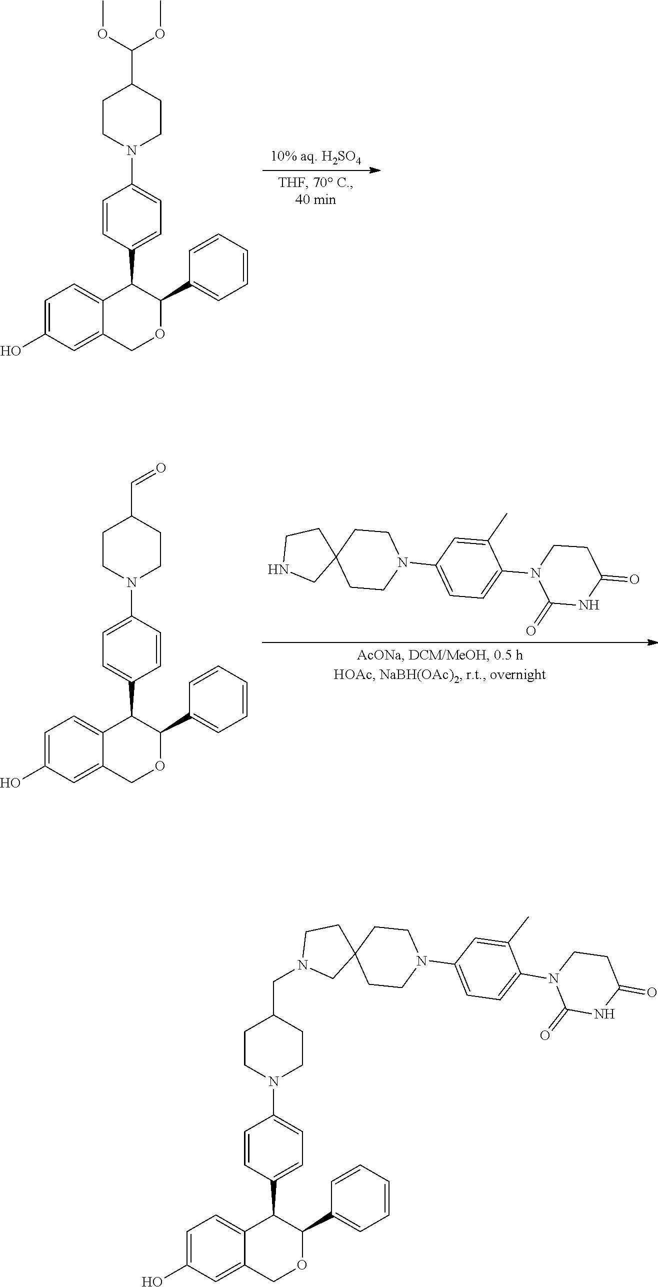 [SMILES: COC(OC)C1CCN(c2ccc([C@@H]3c4ccc(O)cc4CO[C@@H]3c3ccccc3)cc2)CC1 ; O=CC1CCN(c2ccc([C@@H]3c4ccc(O)cc4CO[C@@H]3c3ccccc3)cc2)CC1 ; Cc1cc(N2CCC3(CCNC3)CC2)ccc1N1CCC(=O)NC1=O ; Cc1cc(N2CCC3(CCN(CC4CCN(c5ccc([C@@H]6c7ccc(O)cc7CO[C@@H]6c6ccccc6)cc5)CC4)C3)CC2)ccc1N1CCC(=O)NC1=O]

Step 1: To a solution of (3S,4R)-4-(4-(4-(dimethoxymethyl)piperidin-1-yl)phenyl)-3-phenylisochroman-7-ol (100 mg, 1 Eq, 218 μmol) in 10% H₂SO₄ (5 mL) and THF (3 mL) was stirred at 70° C. for 40 min. The reaction was adjust to pH=˜8 with saturated aq. NaHCO₃ (20 mL). Then the mixture was added water (30 mL) and extracted with ethyl acetate (2×30 mL). The combined organic layers were washed with brine (30 mL), dried over anhydrous Na₂SO₄, filtered and concentrated to dryness in vacuum to give 1-(4-((3S,4R)-7-hydroxy-3-phenylisochroman-4-yl)phenyl)piperidine-4-carbaldehyde (80 mg, 0.18 mmol, 95.55% purity) as a yellow solid. LC-MS (ESI⁺) m/z: 432.1 (M+H₂O)⁺.

Step 2: To a solution of 1-(4-((3S,4R)-7-hydroxy-3-phenylisochroman-4-yl)phenyl)piperidine-4-carbaldehyde (100 mg, 1 Eq, 231.07 μmol), 1-(2-methyl-4-(2,8-diazaspiro[4.5]decan-8-yl)phenyl)dihydropyrimidine-2,4 (1H,3H)-dione hydrochloride (88 mg, 1 Eq, 231.07 μmol) in DCM (3 mL) and MeOH (3 mL) was added sodium acetate (114 mg, 6 Eq, 1.3864 mmol) was stirred at 25° C. for 1 h, Then acetic acid (28 mg, 2 Eq, 462.13 μmol) and sodium triacetoxyhydroborate (98 mg, 2 Eq, 462.13 μmol) was added. The mixture was stirred at 30° C. for 16 hour. The reaction was quenched with H₂O (50 mL) and extracted with ethyl acetate (50 mL*2). The organic layer was washed with brine (30 mL) and dried over anhydrous Na₂SO₄ and concentrated in vacuo to give a yellow oil. The residue was purified by prep-HPLC (column: Welch Xtimate C18 150*30 mm*3 um; mobile phase: [water (FA)-ACN]; B % 11%-41%, 7 min) to give 1-(4-(2-((1-(4-((3S,4R)-7-hydroxy-3-phenylisochroman-4-yl)phenyl)piperidin-4-yl)methyl)-2,8-diazaspiro[4.5]decan-8-yl)-2-methylphenyl)dihydropyrimidine-2,4 (1H,3H)-dione (97.8 mg, 132 μmol, 100% purity) as a white solid. LC-MS (ESI⁺) m/z: 740.5 (M+H)⁺.

LCMS: calc. for C₄₆H₅₃N₅O₄: 739.41, found: [M+H]+ 740.5.

HPLC: 100% purity at 220 nm.

¹H NMR (400 MHZ, DMSO-d₆) δ=10.24 (s, 1H), 9.57-9.07 (m, 1H), 8.18 (s, 0.491H), 7.15-7.02 (m, 6H), 6.83-6.74 (m, 3H), 6.62-6.58 (m, 2H), 6.58-6.52 (m, 4H), 5.09-4.88 (m, 3H), 4.02 (br d, J=2.4 Hz, 1H), 3.68-3.62 (m, 1H), 3.45 (br d, J=5.8 Hz, 2H), 3.20-3.04 (m, 5H), 2.76-2.58 (m, 3H), 2.47-2.40 (m, 2H), 2.35 (s, 2H), 2.24 (br d, J=7.0 Hz, 2H), 2.10 (s, 3H), 1.73 (br d, J=12.0 Hz, 2H), 1.66-1.42 (m, 8H), 1.19-1.07 (m, 2H).

### Example 108. Preparation of (I-367) (S)-3-(5-(4-((1-(4-((1R,3S,4R)-7-hydroxy-1-methyl-3-phenylisochroman-4-yl)phenyl)piperidin-4-yl)methyl)piperazin-1-yl)-1-oxoisoindolin-2-yl)piperidine-2,6-dione

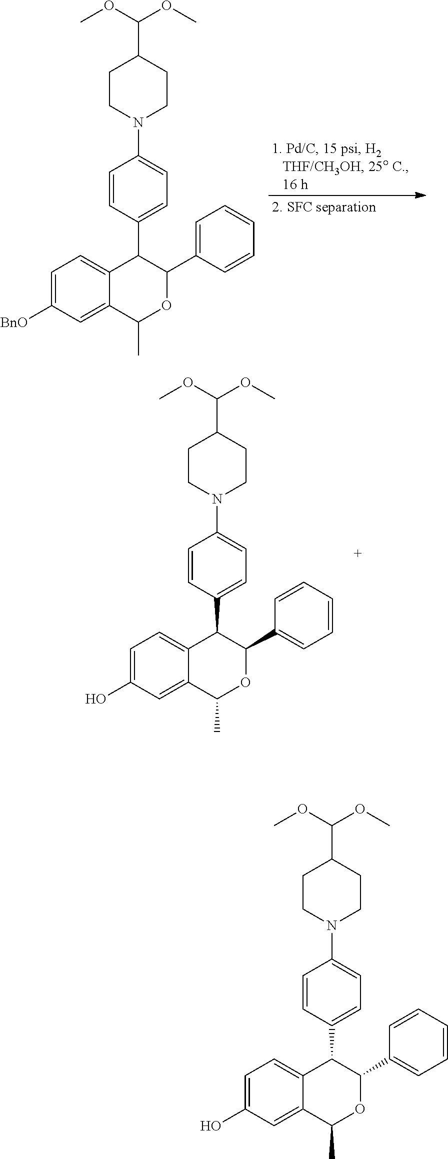 [SMILES: COC(OC)C1CCN(c2ccc(C3c4ccc(OCc5ccccc5)cc4C(C)OC3c3ccccc3)cc2)CC1 ; COC(OC)C1CCN(c2ccc([C@@H]3c4ccc(O)cc4[C@@H](C)O[C@@H]3c3ccccc3)cc2)CC1 ; COC(OC)C1CCN(c2ccc([C@H]3c4ccc(O)cc4[C@H](C)O[C@H]3c3ccccc3)cc2)CC1]

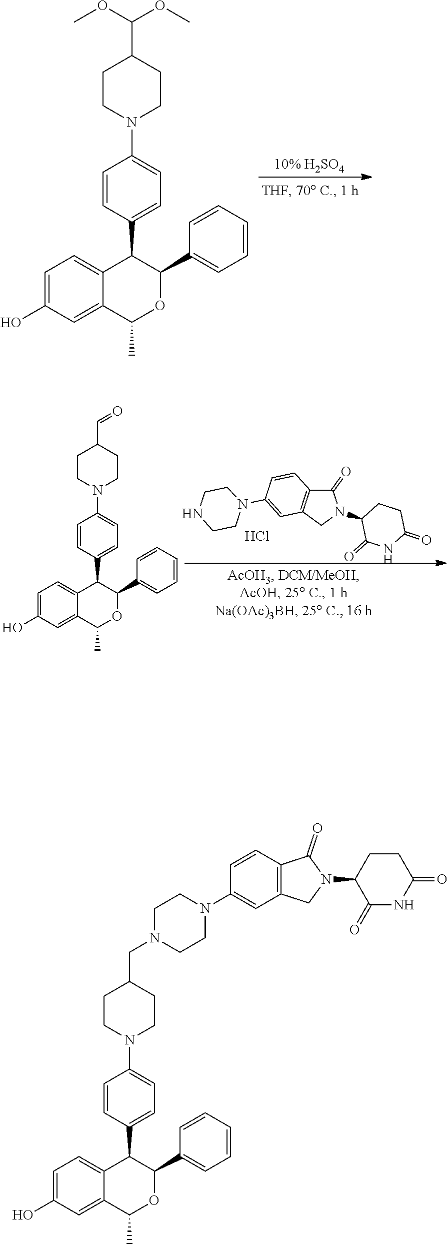 [SMILES: COC(OC)C1CCN(c2ccc([C@@H]3c4ccc(O)cc4[C@@H](C)O[C@@H]3c3ccccc3)cc2)CC1 ; C[C@H]1O[C@H](c2ccccc2)[C@H](c2ccc(N3CCC(C=O)CC3)cc2)c2ccc(O)cc21 ; O=C1CC[C@H](N2Cc3cc(N4CCNCC4)ccc3C2=O)C(=O)N1 ; Cl ; C[C@H]1O[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21]

Step 1: A mixture of 1-(4-(7-(benzyloxy)-1-methyl-3-phenylisochroman-4-yl)phenyl)-4-(dimethoxymethyl)piperidine (130 mg, 1 Eq, 231 μmol), Pd/C (130 mg, 10% purity, 0.53 Eq, 122 μmol) was added in THF (3 mL) and MeOH (3 mL). The suspension was degassed and purged with H₂ for 3 times. The mixture was stirred under H₂ (15 psi) at 25° C. for 16 hour. The mixture was filtered and concentrated to dryness in vacuum to give a white solid. The white solid was subjected by SFC: Column: DAICEL CHIRALPAK AD (250 mm*30 mm, 10 um); Mobile phase: A: Supercritical CO₂, B: MeOH (0.1% NH₃H₂O), A:B=50:50 at 80 mL/min. The pure fractions were collected respectively and the solvents were evaporated under vacuum to give (1S,3R,4S)-4-(4-(4-(dimethoxymethyl)piperidin-1-yl)phenyl)-1-methyl-3-phenylisochroman-7-ol (42 mg, 85 μmol, 37% yield, 96.08% purity) and (1R,3S,4R)-4-(4-(4-(dimethoxymethyl)piperidin-1-yl)phenyl)-1-methyl-3-phenylisochroman-7-ol (47 mg, 97 μmol, 42% yield, 97.83% purity) as a yellow solid.

LC-MS (ESI⁺) m/z: 474.1 (M+H)⁺.

Step 2: A mixture of (1R,3S,4R)-4-(4-(4-(dimethoxymethyl)piperidin-1-yl)phenyl)-1-methyl-3-phenylisochroman-7-ol (47 mg, 97.831% purity, 1 Eq, 97 μmol) in THF (1.5 mL) and 10% H₂SO₄ (1.5 mL) was stirred at 70° C. for 1 hour. Then the reaction was cooled to room temperature. The reaction mixture was quenched with saturated aq. NaHCO₃ (5 mL) and stirred for 10 min at 0° C. Then the mixture was dissolved in water (50 mL) and extracted by ethyl acetate (50 mL*2). The combined organic layer were dried over anhydrous Na₂SO₄, filtered and concentrated under vacuum to give 1-(4-((1R,3S,4R)-7-hydroxy-1-methyl-3-phenylisochroman-4-yl)phenyl)piperidine-4-carbaldehyde (40 mg, 94 μmol, 96% yield) as a yellow oil.

LC-MS (ESI⁺) m/z: 428.2 (M+H)⁺

Step 3: A mixture of 1-(4-((1R,3S,4R)-7-hydroxy-1-methyl-3-phenylisochroman-4-yl)phenyl)piperidine-4-carbaldehyde (40 mg, 1 Eq, 94 μmol), (S)-3-(1-oxo-5-(piperazin-1-yl)isoindolin-2-yl)piperidine-2,6-dione hydrochloride (44 mg, 1.3 Eq, 0.12 mmol), sodium acetate (38 mg, 5 Eq, 0.47 mmol) and acetic acid (17 mg, 16 μL, 3 Eq, 0.28 mmol) in DCM (2 mL) and MeOH (2 mL) stirred at 25° C. for 1 hour, then sodium triacetoxyborohydride (40 mg, 2 Eq, 0.19 mmol) was added to the mixture and stirred at 25° C. for 16 hour. The reaction was treated with H₂O (50 mL), extracted with ethyl acetate (50 mL). The extracts was dried over anhydrous Na₂SO₄, filtered and concentrated to dryness in vacuum to give a residue. The residue was purified by prep.HPLC (column: Welch Xtimate C18 150*30 mm*5 um mobile phase: [water (FA)-ACN]; B %: 18%-48%, 7 min). The aqueous phase was lyophilized to dryness to give (S)-3-(5-(4-((1-(4-((1R,3S,4R)-7-hydroxy-1-methyl-3-phenylisochroman-4-yl)phenyl)piperidin-4-yl)methyl)piperazin-1-yl)-1-oxoisoindolin-2-yl)piperidine-2,6-dione (24.7 mg, 32.7 μmol, 35% yield, 97.854% purity) as a white solid.

LC-MS (ESI⁺) m/z: 740.2 (M+H)⁺

HPLC: 97.854%, purity at 220 nm.

¹H NMR (400 MHZ, DMSO-d₆) δ=10.96 (s, 1H), 9.49-9.11 (m, 1H), 8.20 (s, 0.306H), 7.52 (d, J=8.6 Hz, 1H), 7.18-7.04 (m, 7H), 6.78 (d, J=8.1 Hz, 1H), 6.62-6.51 (m, 6H), 5.32 (q, J=6.8 Hz, 1H), 5.25 (d, J=2.7 Hz, 1H), 5.05 (dd, J=5.0, 13.2 Hz, 1H), 4.38-4.28 (m, 1H), 4.24-4.17 (m, 1H), 3.98 (d, J=3.0 Hz, 1H), 3.55-3.47 (m, 3H), 3.27 (br s, 5H), 2.97-2.84 (m, 1H), 2.58 (br d, J=16.1 Hz, 1H), 2.48 (br s, 4H), 2.40-2.30 (m, 1H), 2.18 (br d, J=6.9 Hz, 2H), 2.01-1.91 (m, 1H), 1.74 (br d, J=11.8 Hz, 2H), 1.67-1.58 (m, 1H), 1.53 (d, J=6.7 Hz, 3H), 1.21-1.08 (m, 2H).

### Example 109. Preparation of (I-368) (S)-3-(5-(4-((1-(4-((1S,3R,4S)-7-hydroxy-1-methyl-3-phenylisochroman-4-yl)phenyl)piperidin-4-yl)methyl)piperazin-1-yl)-1-oxoisoindolin-2-yl)piperidine-2,6-dione

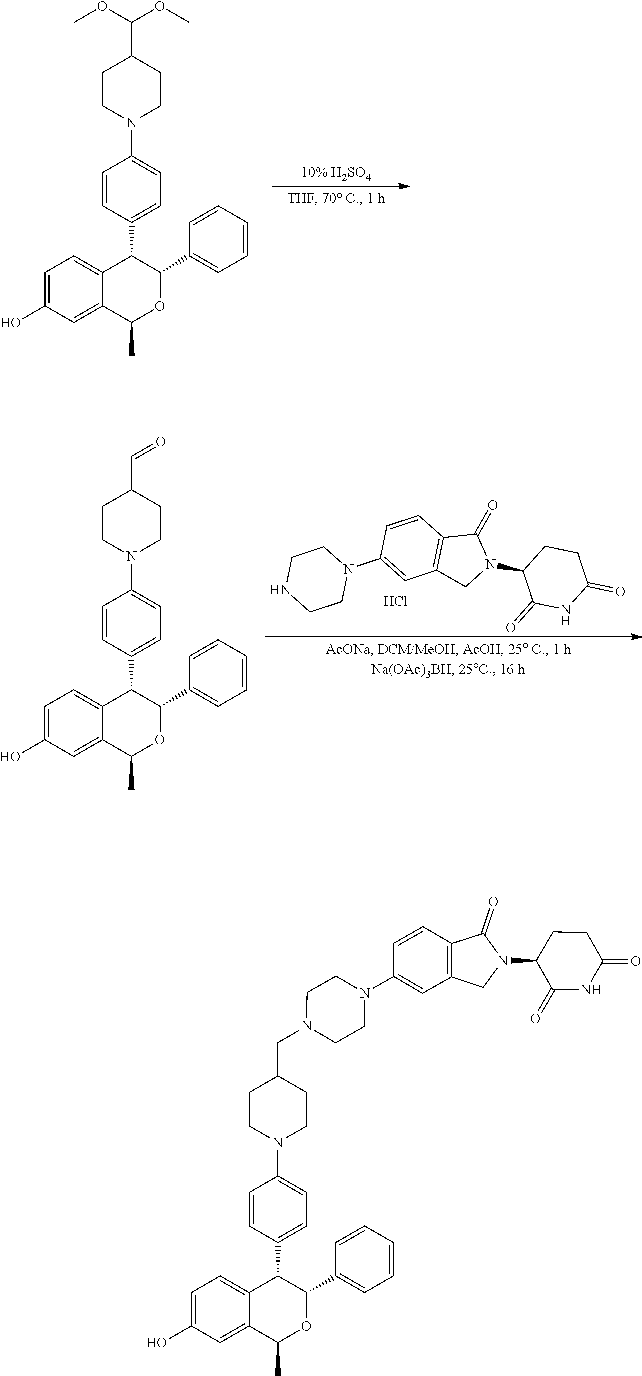 [SMILES: COC(OC)C1CCN(c2ccc([C@H]3c4ccc(O)cc4[C@H](C)O[C@H]3c3ccccc3)cc2)CC1 ; C[C@@H]1O[C@@H](c2ccccc2)[C@@H](c2ccc(N3CCC(C=O)CC3)cc2)c2ccc(O)cc21 ; O=C1CC[C@H](N2Cc3cc(N4CCNCC4)ccc3C2=O)C(=O)N1 ; Cl ; C[C@@H]1O[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21]

Step 1: A mixture of (1*S,3*R,4*S)-4-(4-(4-(dimethoxymethyl)piperidin-1-yl)phenyl)-1-methyl-3-phenylisochroman-7-ol (37 mg, 96.08% purity, 1 Eq, 75 μmol) in THF (1.5 mL) and 10% H₂SO₄ (1.5 mL) was stirred at 70° C. for 1 hour. Then the reaction was cooled to room temperature. The reaction mixture was quenched with saturated aq. NaHCO₃ (5 mL) and stirred for 10 min at 0° C. Then the mixture was dissolved in water (50 mL) and extracted by ethyl acetate (50 mL*2). The combined organic layer were dried over anhydrous Na₂SO₄, filtered and concentrated under vacuum to give 1-(4-((1*S,3*R,4*S)-7-hydroxy-1-methyl-3-phenylisochroman-4-yl)phenyl)piperidine-4-carbaldehyde (32 mg, 75 μmol, 100% yield) as a yellow oil.

LC-MS (ESI⁺) m/z: 428.1 (M+H)⁺

Step 2: A mixture of 1-(4-((1S,3R,4S)-7-hydroxy-1-methyl-3-phenylisochroman-4-yl)phenyl)piperidine-4-carbaldehyde (32 mg, 1 Eq, 75 μmol), (S)-3-(1-oxo-5-(piperazin-1-yl)isoindolin-2-yl)piperidine-2,6-dione hydrochloride (35 mg, 1.3 Eq, 0.097 mmol), sodium acetate (31 mg, 5 Eq, 0.37 mmol) and acetic acid (13 mg, 13 μL, 3 Eq, 0.22 mmol) in DCM (2 mL) and MeOH (2 mL) stirred at 25° C. for 1 hour, then sodium triacetoxyborohydride (32 mg, 2 Eq, 0.15 mmol) was added to the mixture and stirred at 25° C. for 16 hour. The reaction was treated with H₂O (50 mL), extracted with ethyl acetate (50 mL). The extracts was dried over anhydrous Na₂SO₄, filtered and concentrated to dryness in vacuum to give a residue. The residue was purified by prep. HPLC (column: Welch Xtimate C18 150*30 mm*5 um mobile phase: [water (FA)-ACN]; B %: 19%-49%, 7 min). The aqueous phase was lyophilized to dryness to give (S)-3-(5-(4-((1-(4-((1S,3R,4S)-7-hydroxy-1-methyl-3-phenylisochroman-4-yl)phenyl)piperidin-4-yl)methyl)piperazin-1-yl)-1-oxoisoindolin-2-yl)piperidine-2,6-dione (19.2 mg, 25.9 μmol, 35% yield, 99.820% purity) as a white solid.

LC-MS (ESI⁺) m/z: 740.2 (M+H)⁺

HPLC: 99.8%, purity at 220 nm.

¹H NMR (400 MHZ, DMSO-d₆) δ=10.96 (s, 1H), 9.29 (br s, 1H), 8.19 (s, 0.152H), 7.52 (d, J=8.5 Hz, 1H), 7.18-7.02 (m, 7H), 6.82-6.74 (m, 1H), 6.65-6.52 (m, 6H), 5.38-5.20 (m, 2H), 5.05 (dd, J=5.1, 13.3 Hz, 1H), 4.40-4.27 (m, 1H), 4.26-4.13 (m, 1H), 3.98 (d, J=2.9 Hz, 1H), 3.50 (br d, J=11.4 Hz, 3H), 3.27 (br s, 5H), 2.99-2.81 (m, 1H), 2.58 (br d, J=16.9 Hz, 1H), 2.48 (br s, 4H), 2.37 (br dd, J=4.5, 12.9 Hz, 1H), 2.25-2.13 (m, 2H), 2.01-1.90 (m, 1H), 1.80-1.69 (m, 2H), 1.67-1.59 (m, 1H), 1.56-1.48 (m, 3H), 1.20-1.07 (m, 2H).

### Example 110. Preparation of (I-205) (S)-3-(5-(4-((1-(4-((1*S,3*R,4*R)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione

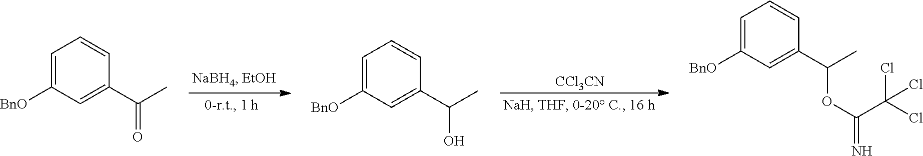 [SMILES: CC(=O)c1cccc(OCc2ccccc2)c1 ; CC(O)c1cccc(OCc2ccccc2)c1 ; "CCl3CN" ; CC(OC(=N)C(Cl)(Cl)Cl)c1cccc(OCc2ccccc2)c1]

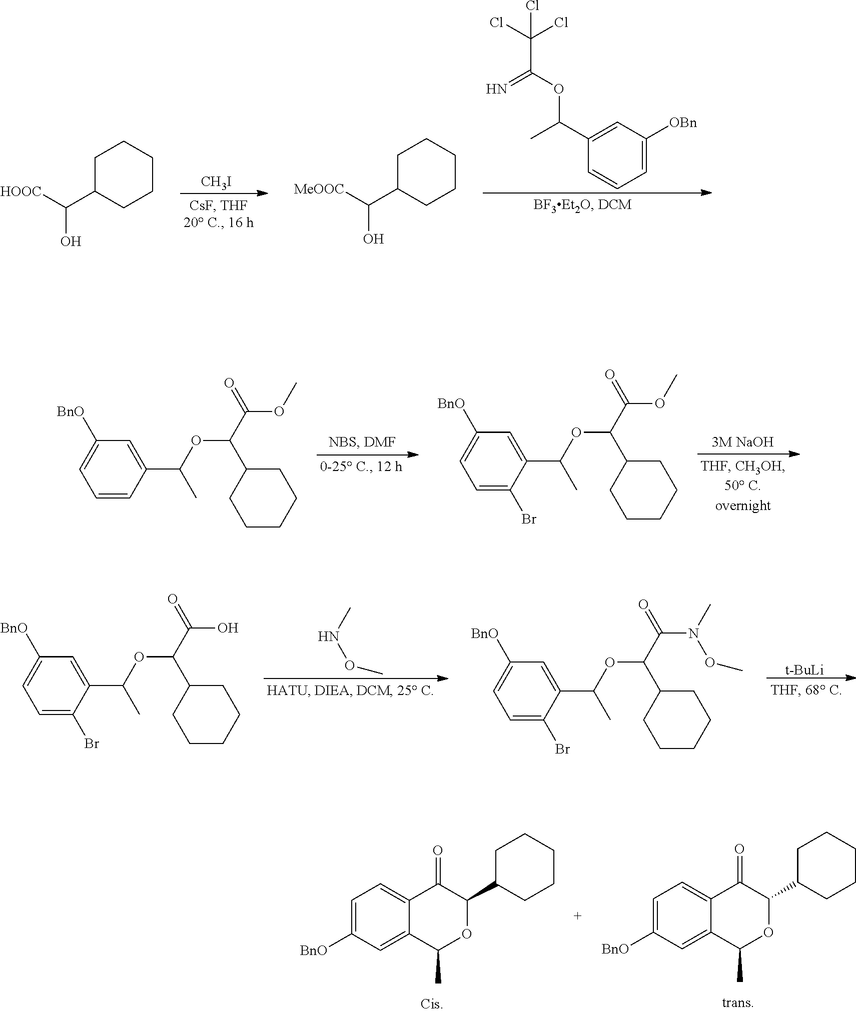

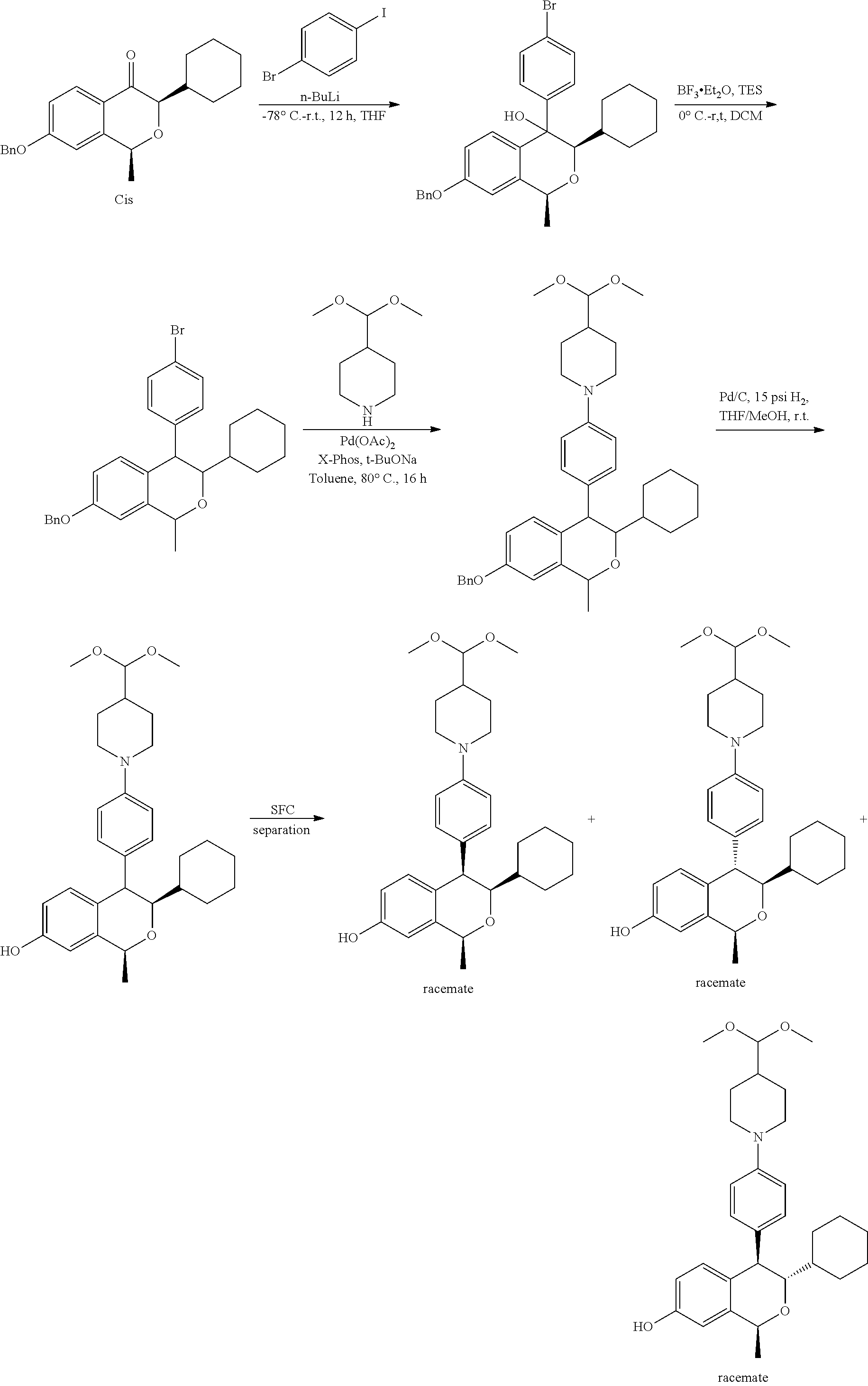

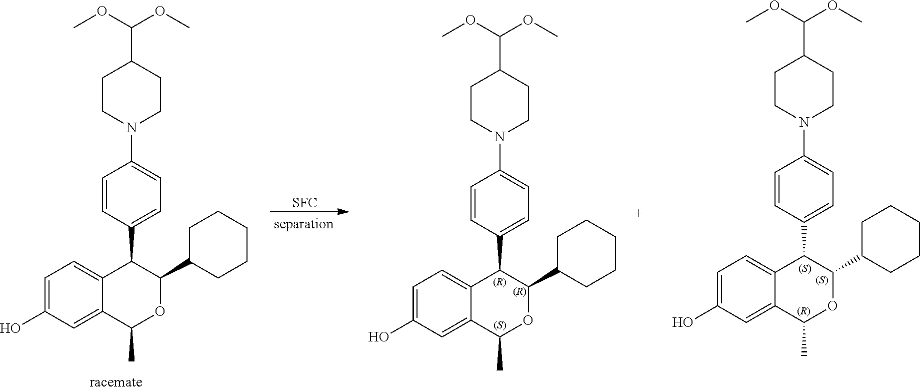

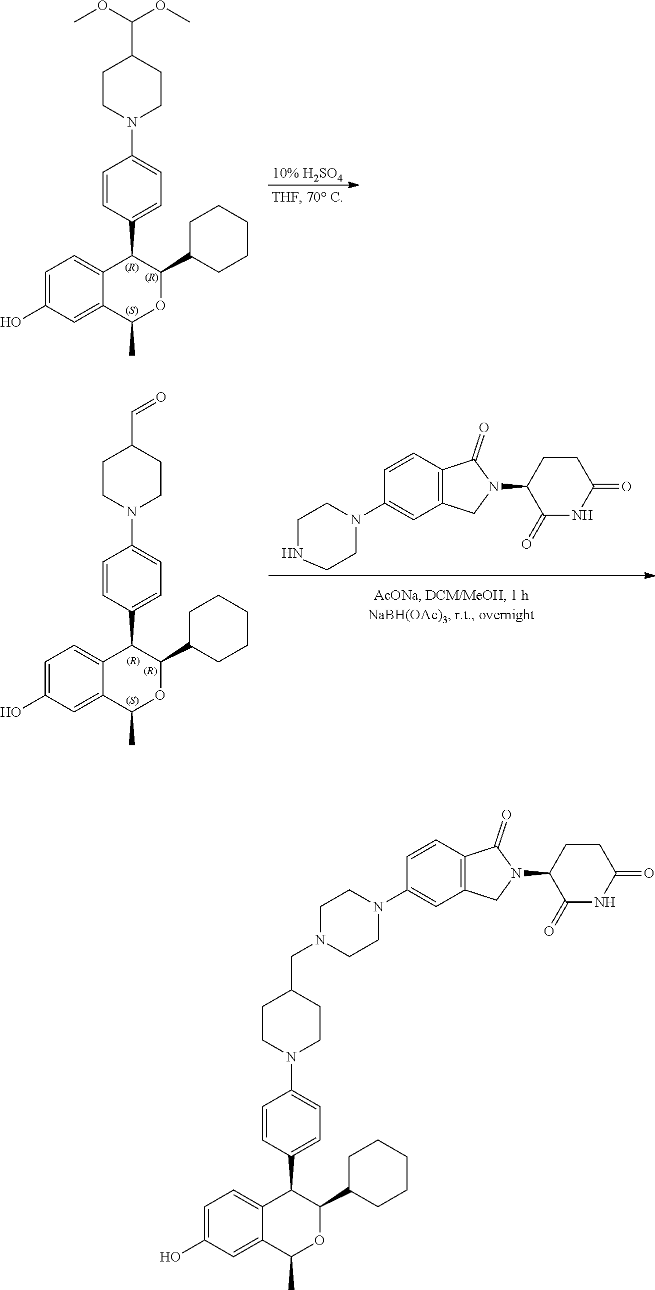

Step 1: A mixture of sodium tetrahydrofolate (8.7 g, 1.04 Eq, 229 mmol) was added in 1-(3-(benzyloxy)phenyl) ethan-1-one (50 g, 1 Eq, 221 mmol) in EtOH (300 mL) with N₂ at 0° C. and was stirred at 0° C. for 1 hour. TLC (petroleum ether:ethyl acetate=5:1, UV) showed one main new spot was observed. The reaction was quenched with saturated aqueous NH₄Cl (80 mL) at 0° C., then mixture was added to H₂O (300 mL) was extracted with ethyl acetate (500 mL*2). The combined organic layers were washed with brine (50 mL), dried over anhydrous Na₂SO₄, filtered and concentrated to dryness in vacuum to give 1-(3-(benzyloxy)phenyl) ethan-1-ol (50 g, 219 mmol, 99.1%) as a yellow solid.

Step 2: A mixture of 1-(3-(benzyloxy)phenyl) ethan-1-ol (20 g, 1 Eq, 87.61 mmol) in THF (50 mL) were added to a 250 mL round-bottomed flask, then cooled to 0° C., NaH (4.2 g, 60% purity, 1.2 Eq, 105.1 mmol) was added. The mixture stirred under N₂ at 0° C. for 30 min. Then 2, 2, 2-trichloroacetonitrile (13.9 g, 9.7 mL, 1.1 Eq, 96.37 mmol) was added dropwise and the reaction was stirred at 20° C. for 16 hour. TLC (petroleum ether:ethyl acetate=10:1, UV) showed one main new spot was observed. 300 mL water was added, and the mixture was extracted with dichloromethane (500 mL*2). The combined organic layers were washed with brine (200 mL), dried over anhydrous Na₂SO₄, filtered and concentrated to dryness in vacuum to give a yellow oil. The yellow oil was purified by flash column (petroleum ether/ethyl acetate from 100/0 to 0/100) and the organic layer was concentrated in vacuum to give 1-(3-(benzyloxy)phenyl)ethyl 2, 2, 2-trichloroacetimidate (9 g, 24 mmol, 28% yield) as a yellow solid. NMR confirmed.

Step 3: A mixture of 2-cyclohexyl-2-hydroxyacetic acid (10 g, 1 Eq, 63.2 mmol), cesium fluoride (14.4 g, 3.5 mL, 1.5 Eq, 94.8 mmol) and methyl iodide (13.5 g, 6 mL, 1.5 Eq, 94.8 mmol) in DMF (80 mL) was stirred at 20° C. for 16 hour. TLC (petroleum ether:ethyl acetate=5:1, R_{f}=0.5) showed a new spot was formed. The mixture was added H₂O (300 mL) and extracted with ethyl acetate (200 mL*2). The combined extracts was dried over anhydrous Na₂SO₄, filtered and concentrated to dryness in vacuum to give methyl 2-cyclohexyl-2-hydroxyacetate (8 g, 4 mmol) as a yellow oil.

Step 4: To a solution of 1-(3-(benzyloxy)phenyl)ethyl 2, 2, 2-trichloroacetimidate (6.2 g, 1 Eq, 16.6 mmol) in DCM (30 mL) was under N₂ atmosphere. A freshly prepared solution of methyl 2-cyclohexyl-2-hydroxyacetate (3.5 g, 1.2 Eq, 20 mmol) was added. After cooling at 0° C. boron difluoride etherate (236 mg, 208 μL, 0.1 Eq, 1.66 mmol) was added stirred at 0° C., and the solution was allowed to reach 0° C. during 2 h under stirring while developing a white precipitate. TLC (petroleum ether:ethyl acetate=10:1, UV) showed one main new spot was observed. 100 mL water was added, and the mixture was extracted with dichloromethane (300 mL*2). The combined organic layers were washed with brine (50 mL), dried over anhydrous Na₂SO₄, filtered and concentrated to dryness in vacuum to give a yellow oil. The yellow oil was purified by preparative high-performance liquid chromatography. Condition: Column: Welch Xtimate C18 150*30 mm*5 um A: water (FA); B: ACN; at the beginning: A (30%) and B (70%); at the end: A: (0%) and B (100%) Gradient Time (min) 9; 100% B hold Time (min) 3, Flow Rate (ml/min) 30. The pure fractions were collected and the solvent was evaporated under vacuum. The aqueous layer was lyophilized to dryness to give methyl 2-(1-(3-(benzyloxy)phenyl)ethyl)-2-cyclohexylacetate (1.1 g, 2.9 mmol, 17% yield) as a yellow solid. NMR confirmed.

Step 5: To a mixture of methyl 2-(1-(3-(benzyloxy)phenyl)ethyl)-2-cyclohexylacetate (1.5 g, 1 Eq, 3.92 mmol) and 1-bromopyrrolidine-2,5-dione (942 mg, 1.35 Eq, 5.29 mmol) in DMF (20 mL) was stirred at 0° C. for 1 hour, then the mixture was stirred at 25° C. for 12 hour. TLC (petroleum ether:ethyl acetate=10:1, UV) showed one main new spot was observed. Water (100 mL) was added and extracted with ethyl acetate (250 mL*2). The organic layers were washed with brine (100 mL) and dried over anhydrous Na₂SO₄ and concentrated in vacuum to give a yellow solid. The yellow solid was subjected to column chromatography over silica gel (gradient elution: 0-15% EtOAc). The desired fractions were collected and concentrated to dryness in vacuum to give methyl 2-(1-(5-(benzyloxy)-2-bromophenyl)ethyl)-2-cyclohexylacetate (1.62 g, 3.51 mmol, 89.5% yield) as a white solid.

Step 6: A solution of sodium hydroxide (694 mg, 5.78 mL, 3 molar, 5 Eq, 17.3 mmol) was added to methyl 2-(1-(5-(benzyloxy)-2-bromophenyl)ethyl)-2-cyclohexylacetate (1.6 g, 1 Eq, 3.47 mmol) in MeOH (10 mL) and THF (10 mL) was stirred at 50° C. for 16 hour. 100 mL water was added, and the mixture was extracted with dichloromethane (150 mL*2). The combined organic layers were washed with brine (100 mL), dried over anhydrous Na₂SO₄, filtered and concentrated to dryness in vacuum to give a yellow oil. TLC (petroleum ether:ethyl acetate=5:1, UV) showed one main new spot was observed. The yellow oil was purified by flash column (20 g) (petroleum ether/ethyl acetate from 100/0 to 0/100) and the organic layer was concentrated in vacuum to give 2-(1-(5-(benzyloxy)-2-bromophenyl)ethyl)-2-cyclohexylacetic acid (1.2 g, 2.7 mmol, 77% yield) as a yellow solid.

Step 7: To a solution of on-dimethyl-hydroxylaminehcl (0.29 g, 1.1 Eq, 3.0 mmol) and 2-(1-(5-(benzyloxy)-2-bromophenyl) ethoxy)-2-cyclohexylacetic acid (1.2 g, 1 Eq, 2.7 mmol) in DCM (15 mL) was added HATU (1.5 g, 1.5 Eq, 4.0 mmol) and Diisopropylethylamine (1 g, 1.4 mL, 3 Eq, 8 mmol). The mixture was stirred at 25° C. for 12 hour. TLC (petroleum ether:ethyl acetate=5:1, R_{f}=0.5 UV) showed one main new spot was observed. The reaction was quenched with water (100 mL) and extracted with ethyl acetate (150 mL*2). The organic layer was washed with brine (100 mL) and dried over anhydrous Na₂SO₄ and concentrated in vacuum to give a white solid. The white solid was subjected to column chromatography over silica gel (gradient elution: 0-30% EtOAc). The desired fractions were collected, and concentrated to dryness in vacuum to give 2-(1-(5-(benzyloxy)-2-bromophenyl) ethoxy)-2-cyclohexyl-N-methoxy-N-methylacetamide (1.15 g, 2.34 mmol, 87% yield) as a white solid.

Step 8: A solution of 2-(1-(5-(benzyloxy)-2-bromophenyl) ethoxy)-2-cyclohexyl-N-methoxy-N-methylacetamide (1 g, 1 Eq, 2.039 mmol) in THF (15 mL) was stirred at −68° C. for 10 min under N₂ atmosphere, then added tert-butyllithium (3 mL, 1.3 M, 2 Eq, 4.078 mmol) at −68° C. The reaction was stirred at −68° C. to 25° C. for 16 hour under N₂ atmosphere. TLC (petroleum ether:ethyl acetate=5:1, UV) showed one main new spot was observed. The reaction was quenched with saturated aqueous NH₄Cl (40 mL) and extracted with ethyl acetate (100 mL*3). The organic layer was washed with brine (100 mL) and dried over anhydrous Na₂SO₄ and concentrated in vacuo to give crude product. The yellow solid was purified by preparative high-performance liquid chromatography. Condition: Column: Welch Xtimate C18 150*30 mm*5 um; A: water (FA); B: ACN; at the beginning: A (20%) and B (80%); at the end: A: (0%) and B (100%); Gradient Time (min) 9; 100% B hold Time (min) 2, Flow Rate (ml/min) 30. The pure fractions were collected and the solvent was evaporated under vacuum. The aqueous layer was lyophilized to dryness to give cis-7-(benzyloxy)-3-cyclohexyl-1-methylisochroman-4-one (300 mg, 856 μmol, 41.98% yield) and trans-7-(benzyloxy)-3-cyclohexyl-1-methylisochroman-4-one (170 mg, 485.1 μmol, 23.79% yield) as a yellow solid.

Step 9: To a solution of 1-bromo-4-iodobenzene (403 mg, 2 Eq, 1.4267 mmol) in THE (8 mL) was added n-butyl lithium (428 μL, 2.5 M, 1.5 Eq, 1.07 mmol) under-78° C., after stirred 30 min was added cis-7-(benzyloxy)-3-cyclohexyl-1-methylisochroman-4-one (250 mg, 1 Eq, 713.35 μmol). The resulting mixture was stirred for-78° C. at 2 hour. Then the mixture was stirred at r.t. for 10 hour. TLC (petroleum ether/ethyl acetate=10/1, R_{f}=0.4) showed new spot was formed. The reaction mixture was quenched by addition of water (20 mL), extracted with ethyl acetate (40 mL*2). The combined organic layers were washed with brine (50 mL), dried over anhydrous Na₂SO₄, filtered and concentrated under reduced pressure to give a residue. The residue was purified by flash silica gel chromatography (from petroleum ether/ethyl acetate=I/O to 3/1, TLC: petroleum ether/ethyl acetate=10/1, R_{f}=0.5) to yield a product. 7-(benzyloxy)-4-(4-bromophenyl)-3-cyclohexyl-1-methylisochroman-4-ol (280 mg, 0.47 mmol, 67% yield, 86% purity) was obtained as yellow oil. LC-MS (ESI⁺) m/z: 491.2 (M−H₂O)⁺.

Step 10: To a solution of boron trifluorideetherate (105 mg, 93 μL, 1.5 Eq, 738.96 μmol) and triethylsilane (115 mg, 157 μL, 2 Eq, 985.28 μmol) was added to a mixture suspension of 7-(benzyloxy)-4-(4-bromophenyl)-3-cyclohexyl-1-methylisochroman-4-ol (250 mg, 1 Eq, 492.64 μmol) in DCM (5 mL) at 0° C. The mixture was stirred at 0° C. for 2 hour. TLC (petroleum ether: ethyl acetate=5:1, UV) showed one main new spot was observed. 50 mL water was added, and the mixture was extracted with dichloromethane (100 mL*2). The reaction was concentrated to dryness in vacuum to give a yellow oil. The yellow oil was purified by flash column (petroleum ether/ethyl acetate from 100/0 to 0/100) and the organic layer was concentrated in vacuum to give 7-(benzyloxy)-4-(4-bromophenyl)-3-cyclohexyl-1-methylisochromane (220 mg, 447.6 μmol, 90.86% purity) as a yellow solid.

Step 11: A mixture of 7-(benzyloxy)-4-(4-bromophenyl)-3-cyclohexyl-1-methylisochromane (220 mg, 1 Eq, 447.6 μmol), 4-(dimethoxymethyl)piperidine (107 mg, 126 μL, 1.5 Eq, 671.5 μmol), palladium (II) acetate (15 mg, 0.15 Eq, 67.15 μmol), dicyclohexyl(2′,4′,6′-triisopropyl-[1,1′-biphenyl]-2-yl)phosphane (43 mg, 0.2 Eq, 89.53 μmol), and sodium 2-methylpropan-2-olate (64 mg, 1.5 Eq, 671.5 μmol) in toluene (5 mL) was heated to 90° C. stirred for 16 hour. TLC (petroleum ether:ethyl acetate=10:1, UV) showed one main new spot was observed. 50 mL water was added, and the mixture was extracted with dichloromethane (100 mL*2). The combined organic layers were washed with brine (50 mL), dried over anhydrous Na₂SO₄, filtered and concentrated to dryness in vacuum to give a yellow oil. The residue was purified by silica gel chromatography, 24 g column, and eluted with 0-50% EtOAc. The selected fractions were combined and concentrated in vacuum to give 1-(4-(7-(benzyloxy)-3-cyclohexyl-1-methylisochroman-4-yl)phenyl)-4-(dimethoxymethyl) piper dine (170 mg, 298 μmol, 66.7% yield, 100% purity) as a white solid.

Step 12: To a solution of 1-(4-(7-(benzyloxy)-3-cyclohexyl-1-methylisochroman-4-yl)phenyl)-4-(dimethoxymethyl)piperidine (170 mg, 1 Eq, 298.4 μmol) in MeOH (3 mL) and THF (3 mL) was added Pd/C (170 mg, 10% Wt., 0.535 Eq, 160 μmol) under N₂ atmosphere. The suspension was degassed and purged with H₂ (15 psi) for 3 mines. The mixture was stirred under H₂ (15 Psi) at r.t. for 16 hr. The reaction was filtered and concentrated under reduced pressure to give a residue. The residue was concentrated under reduced pressure to give desired compound as a yellow oil, which was further separated by SFC (condition: column: Welch Xtimate C18 150*25 mm*5 um); mobile phase: water(NH₄HCO₃)-ACN; B %: 85%˜100%) to give (1S,3R,4R)-3-cyclohexyl-4-(4-(4-(dimethoxymethyl)piperidin-1-yl)phenyl)-1-methylisochroman-7-ol (50.0 mg, 104 μmol, 34.9% yield, racemate) and (1S,3R,4S)-3-cyclohexyl-4-(4-(4-(dimethoxymethyl)piperidin-1-yl)phenyl)-1-methylisochroman-7-ol (30.0 mg, 62.5 μmol, 21.0% yield, racemate) and (1S,3S,4R)-3-cyclohexyl-4-(4-(4-(dimethoxymethyl)piperidin-1-yl)phenyl)-1-methylisochroman-7-ol (20 mg, 41.7 μmol, 14% yield, racemate) as a white solid. LC-MS (ESI⁺) m/z: 480.3 (M+H)⁺.

Step 13: The mixture from step 12 was concentrated under reduced pressure to give desired compound as a yellow oil, which was further separated by SFC (condition: column: DAICEL CHIRALPAK AD (250 mm*30 mm, 10 um)); mobile phase: CO₂-i-PrOH (0.1% NH₃H₂O; B %: 40%-40%) to give (1*S,3*R,4*R)-3-cyclohexyl-4-(4-(4-(dimethoxymethyl)piperidin-1-yl)phenyl)-1-methylisochroman-7-ol (23 mg, 48 μmol, 46% yield) and (1*R,3*S,4*S)-3-cyclohexyl-4-(4-(4-(dimethoxymethyl)piperidin-1-yl)phenyl)-1-methylisochroman-7-ol (22 mg, 45.9 μmol, 44.0% yield) and as a white solid.

Step 14:1-(4-((1*S,3*R,4*R)-3-cyclohexyl-7-hydroxy-1-methylisochroman-4-yl)phenyl)piperidine-4-carbaldehyde (18 mg, 40.4 μmol, 96.8% purity) in sulfuric acid (41 mg, 2 mL, 10% V/V, 1 Eq, 41.7 μmol) and THF (2 mL) was stirred at 70° C. for 2 hour. The reaction was adjust to pH=˜7 with NaHCO₃, then 20 mL water was added, and the mixture was extracted with dichloromethane (20 mL*2). The combined organic layers were washed with brine (10 mL), dried over anhydrous Na₂SO₄, filtered and concentrated to dryness in vacuum to give a yellow oil. The yellow oil was concentrated in vacuum to give 1-(4-((1*S, 3*R, 4*R)-3-cyclohexyl-7-hydroxy-1-methylisochroman-4-yl)phenyl)piperidine-4-carbaldehyde (17.5 mg, 40.4 μmol, 96.8% yield) as a yellow solid. LC-MS (ESI⁺) m/z: 452.4 (M+H₂O)

Step 15: To a solution of 1-(4-(1*S,3*R,4*R)-3-cyclohexyl-7-hydroxy-1-methylisochroman-4-yl)phenyl)piperidine-4-carbaldehyde (17 mg, 90% purity, 1 Eq, 35.3 μmol), (S)-3-(1-oxo-5-(piperazin-1-yl)isoindolin-2-yl)piperidine-2,6-dione (14 mg, 1.1 Eq, 38.8 μmol, HCl salt) in DCM (2 mL), MeOH (2 mL) was added sodium acetate (9 mg, 3 Eq, 106 μmol) was stirred at r.t. for 1 h, Then sodium triacetoxyhydroborate (15 mg, 2 Eq, 70.6 μmol) and acetic acid (6 mg, 6 μL, 3 Eq, 106 μmol) was added. The mixture was stirred at 25° C. for 15 hour. The reaction mixture was concentrated under reduced pressure to remove solvent. The residue was purified by prep-HPLC (column: Welch Xtimate C18 100*40 mm*3 um phase: [water (FA)-ACN]; B %: 20%-60%, 9 min to give (S)-3-(5-(4-((1-(4-((1*S,3*R,4*R)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione (19.1 mg, 25.6 μmol, 72.6% yield, 100% purity) as a white solid.

LCMS: calc. for C₄₅H₅₅N₅O₅:745.42, found: [M+H]⁺746.5.

HPLC: 100% purity at 220 nm.

¹H NMR (400 MHz, DMSO-d₆) § 10.94-11.00 (m, 1H) 9.16-9.24 (m, 1H) 8.15 (s, 0.310H) 7.53 (d, J=8.78 Hz, 1H) 7.03-7.13 (m, 4H) 6.70-6.81 (m, 3H) 6.47-6.56 (m, 2H) 5.06 (dd, J=13.30, 5.27 Hz, 1H) 4.72 (q, J=6.19 Hz, 1H) 4.28-4.38 (m, 1H) 4.18-4.25 (m, 1H) 3.75 (br s, 1H) 3.56-3.66 (m, 2H) 3.25-3.34 (m, 7H) 2.82-3.05 (m, 1H) 2.54-2.63 (m, 4H) 2.30-2.48 (m, 2H) 2.18-2.27 (m, 2H) 1.95-2.06 (m, 2H) 1.80 (br d, J=11.04 Hz, 3H) 1.58-1.70 (m, 3H) 1.51-1.57 (m, 4H) 1.15-1.22 (m, 2H) 1.05-1.12 (m, 1H) 0.86-1.03 (m, 5H).

### Example 111. Preparation of (I-255) (S)-3-(5-(4-((1-(4-((1R,3R,4S)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione

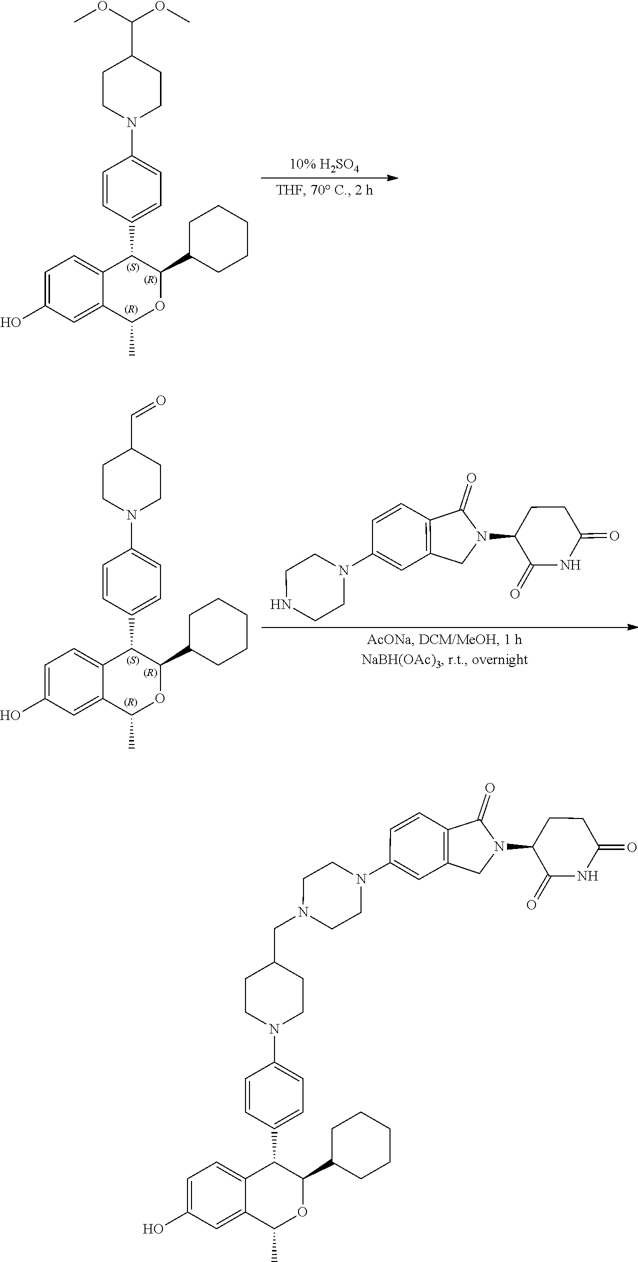

Step 1: A solution of 1-(4-((1R,3R,4S)-3-cyclohexyl-7-hydroxy-1-methylisochroman-4-yl)phenyl)piperidine-4-carbaldehyde (8 mg, 0.02 mmol, 87% purity) in 10% H₂SO₄ (0.02 g, 1 mL, 1 Eq, 0.02 mmol) and THF (1 mL) was stirred at 70° C. for 2 hour. The reaction was adjust to pH=7 with saturated aqueous NaHCO₃, then 50 mL water was added, and the mixture was extracted with dichloromethane (100 mL*2). The combined organic layers were washed with brine (10 mL), dried over anhydrous Na₂SO₄, filtered and concentrated to dryness in vacuo to give a yellow oil. The yellow oil was concentrated in vacuo to give 1-(4-((1R, 3R, 4S)-3-cyclohexyl-7-hydroxy-1-methylisochroman-4-yl)phenyl)piperidine-4-carbaldehyde (8 mg, 0.02 mmol, 90% yield, 87% purity) as a yellow solid.

Step 2: To a solution of 1-(4-((1R,3R,4S)-3-cyclohexyl-7-hydroxy-1-methylisochroman-4-yl)phenyl)piperidine-4-carbaldehyde (8 mg, 87% purity, 1 Eq, 0.02 mmol), (S)-3-(1-oxo-5-(piperazin-1-yl)isoindolin-2-yl)piperidine-2,6-dione (6 mg, 1.1 Eq, 0.02 mmol, HCl salt) in DCM (1 mL), MeOH (1 mL) was added sodium acetate (4 mg, 3 Eq, 0.05 mmol) was stirred at 25° C. for 1 h, Then sodium triacetoxyhydroborate (7 mg, 2 Eq, 0.03 mmol) and acetic acid (3 mg, 3 μL, 3 Eq, 0.05 mmol) was added. The mixture was stirred at 25° C. for 16 hours. The reaction mixture was concentrated under reduced pressure to remove solvent. The residue was purified by prep-HPLC (column: Welch Xtimate C18 150*30 mm*5 um phase: [water (FA)-ACN]; B %: 10%-50%, 9 min to give (S)-3-(5-(4-((1-(4-((1R,3R,4S)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione (2.7 mg, 3.6 μmol, 20% yield, 100% purity) as a white solid.

LCMS: calc. for C₄₅H₅₅N₅O₅: 745.42, found: [M+H]⁺746.5.

HPLC: 100% purity at 220 nm.

¹H NMR (400 MHZ, DMSO-d₆) δ 8.37-8.52 (m, 0.298H) 7.67 (d, J=8.46 Hz, 1H) 7.09-7.15 (m, 2H) 6.95-7.03 (m, 4H) 6.56-6.61 (m, 1H) 6.49-6.54 (m, 2H) 5.08-5.16 (m, 1H) 4.59-4.69 (m, 1H) 4.36-4.49 (m, 2H) 3.93 (d, J=7.75 Hz, 1H) 3.65-3.73 (m, 3H) 3.39-3.46 (m, 4H) 2.88-2.97 (m, 1H) 2.70-2.79 (m, 6H) 2.40-2.54 (m, 3H) 2.16-2.24 (m, 1H) 1.91-1.99 (m, 3H) 1.70-1.85 (m, 3H) 1.64 (br d, J=1.07 Hz, 1H) 1.57 (d, J=6.56 Hz, 3H) 1.29-1.50 (m, 8H) 1.09-1.24 (m, 4H).

The absolute stereochemistry was arbitrarily assigned.

### Example 112. Preparation of (I-364) (S)-3-(5-(4-((1-(4-((3R,4S)-3-(bicyclo[4.2.0]octa-1(6),2,4-trien-3-yl)-7-hydroxyisochroman-4-yl)phenyl)piperidin-4-yl)methyl)piperazin-1-yl)-1-oxoisoindolin-2-yl)piperidine-2,6-dione

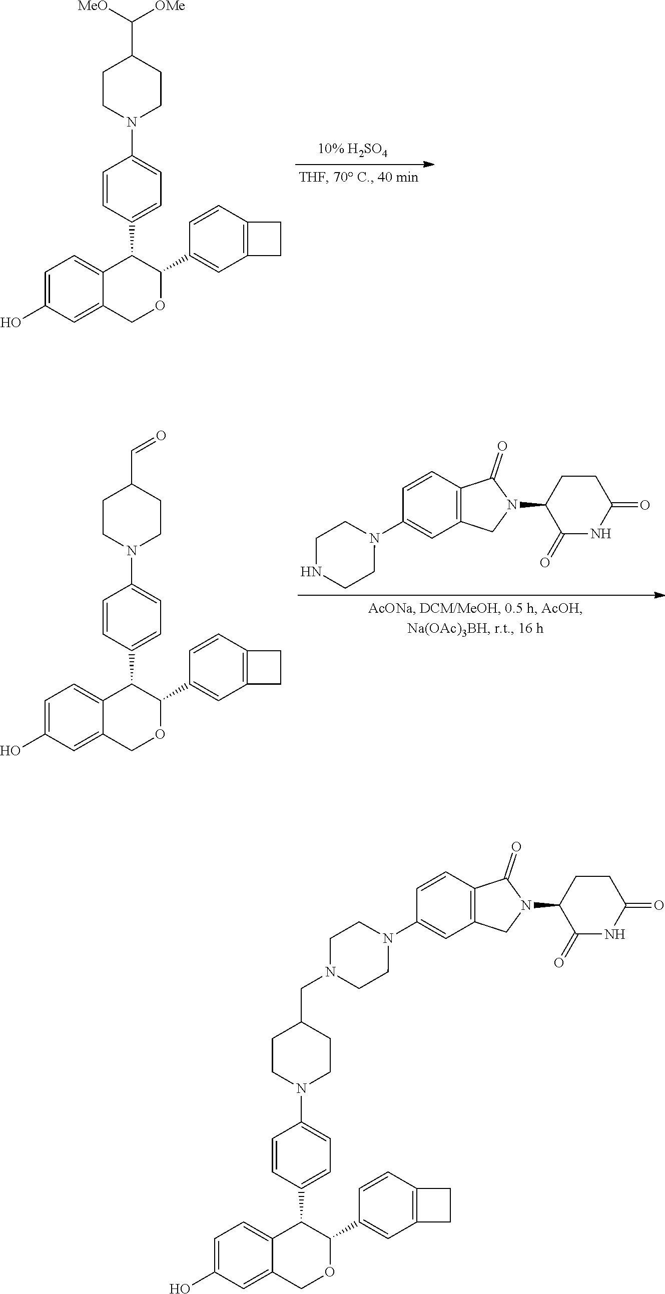 [SMILES: COC(OC)C1CCN(c2ccc([C@H]3c4ccc(O)cc4CO[C@H]3c3ccc4c(c3)CC4)cc2)CC1 ; O=CC1CCN(c2ccc([C@H]3c4ccc(O)cc4CO[C@H]3c3ccc4c(c3)CC4)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CO[C@H]7c7ccc8c(c7)CC8)cc6)CC5)CC4)ccc3C2=O)C(=O)N1]

Step 1: To a solution of (3R,4S)-3-(bicyclo[4.2.0]octa-1(6),2,4-trien-3-yl)-4-(4-(4-(dimethoxymethyl)piperidin-1-yl)phenyl)isochroman-7-ol (54 mg, purity 100%, 1 Eq, 0.11 mmol) in THF (5 mL) was added 10% sulfuric acid (5 mL,) at 0° C. The mixture was stirred at 70° C. for 40 min. The mixture was adjusted to pH=˜8 by adding saturated aq. NaHCO₃ (10 mL) at 0° C. for 10 min. The reaction mixture was diluted with 50 mL H₂O and extracted with 50 mL ethyl acetate. The organic layers were washed with 30 mL brine and dried over anhydrous Na₂SO₄. The organic layers were filtered and concentrated under reduced pressure to give crude product 1-(4-((3R,4S)-3-(bicyclo[4.2.0]octa-1(6),2,4-trien-3-yl)-7-hydroxyisochroman-4-yl)phenyl)piperidine-4-carbaldehyde (44 mg, 99 μmol, 89% yield, 99.2% purity) as a brown oil. LC-MS (ESI⁺) m/z: 440.1.

Step 2: A mixture of 1-(4-(3R,4S)-3-(bicycle[4.2.0]octa-1(6),2,4-trien-3-yl)-7-hydroxyisochroman-4-yl)phenyl)piperidine-4-carbaldehyde (65 mg, 99.2% purity, 1 Eq, 0.15 mmol), (S)-3-(1-oxo-5-(piperazin-1-yl)isoindolin-2-yl)piperidine-2,6-dione hydrochloride (54 mg, 1 Eq, 0.15 mmol) and sodium acetate (60 mg, 5 Eq, 0.73 mmol) were added in DCM (2 mL) and MeOH (2 mL) at 20° C. for 0.5 hour, then added acetic acid (26 mg, 25 μL, 3 Eq, 0.44 mmol) and sodium triacetoxyborohydride (62 mg, 2 Eq, 0.29 mmol, then the mixture was stirred at 25° C. for 16 hour. The reaction was diluted with water (50 mL) and extracted with ethyl acetate (50 mL*2). The combined organic layers were concentrated to give crude product as a brown oil. The brown oil was purified by preparative high-performance liquid chromatography. Condition: water (FA)-ACN. Column: Welch Xtimate C18 150*30 mm*5 um. Begin B: 20%, End B: 50%. Gradient time (min) 7; 100% B hold time (min) 1.7, Flow rate (ml/min) 25. The aqueous layer was lyophilized to dryness to give (S)-3-(5-(4-((1-(4-((3R,4S)-3-(bicyclo[4.2.0]octa-1(6),2,4-trien-3-yl)-7-hydroxyisochroman-4-yl)phenyl)piperidin-4-yl)methyl)piperazin-1-yl)-1-oxoisoindolin-2-yl)piperidine-2,6-dione (23.6 mg, 30.9 μmol, 21% yield, 98.45% purity) as a white solid.

LCMS: calc. for C₄₆H₄₉N₅O₅:751.37, found: [M+H]⁺752.3.

HPLC: 98.46% purity at 220 nm.

¹H NMR (400 MHZ, DMSO-d₆) δ=10.92 (s, 1H), 9.24 (s, 1H), 8.13 (s, 0.278H), 7.51 (d, J=8.3 Hz, 1H), 7.08-7.02 (m, 2H), 6.92-6.88 (m, 1H), 6.85-6.81 (m, 1H), 6.79-6.73 (m, 2H), 6.66-6.62 (m, 2H), 6.60-6.52 (m, 4H), 5.08-5.01 (m, 2H), 4.95-4.85 (m, 2H), 4.36-4.29 (m, 1H), 4.24-4.17 (m, 1H), 3.98 (brd, J=1.9 Hz, 1H), 3.52 (br d, J=11.6 Hz, 2H), 3.29-3.23 (m, 4H), 3.04-2.85 (m, 5H), 2.58 (br d, J=17.6 Hz, 4H), 2.48-2.44 (m, 2H), 2.44-2.30 (m, 2H), 2.20 (brd, J=1.1 Hz, 2H), 2.00-1.92 (m, 1H), 1.75 (br d, J=12.3 Hz, 2H), 1.69-1.60 (m, 1H), 1.21-1.09 (m, 2H).

### Example 113. Preparation of (I-252) (S)-3-(5-(4-((1-(4-((1S,3R,4S)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione

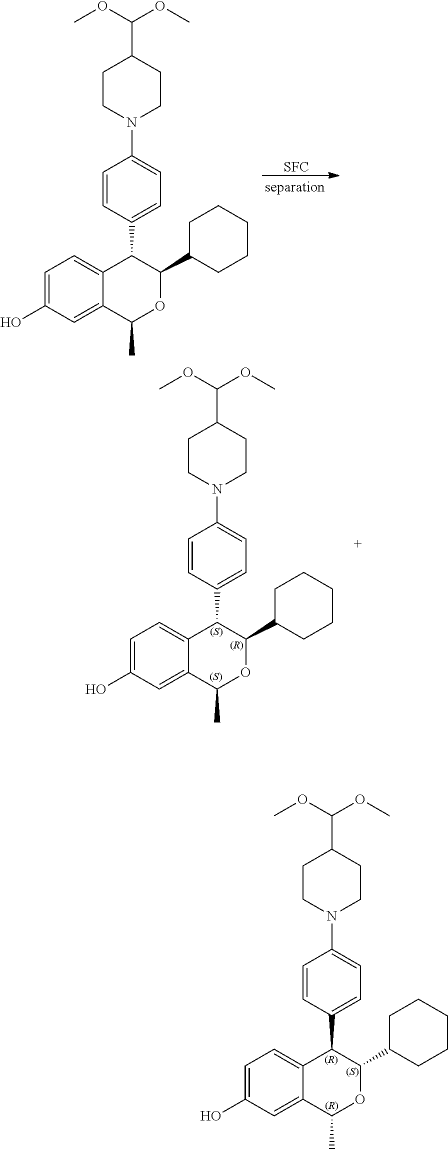

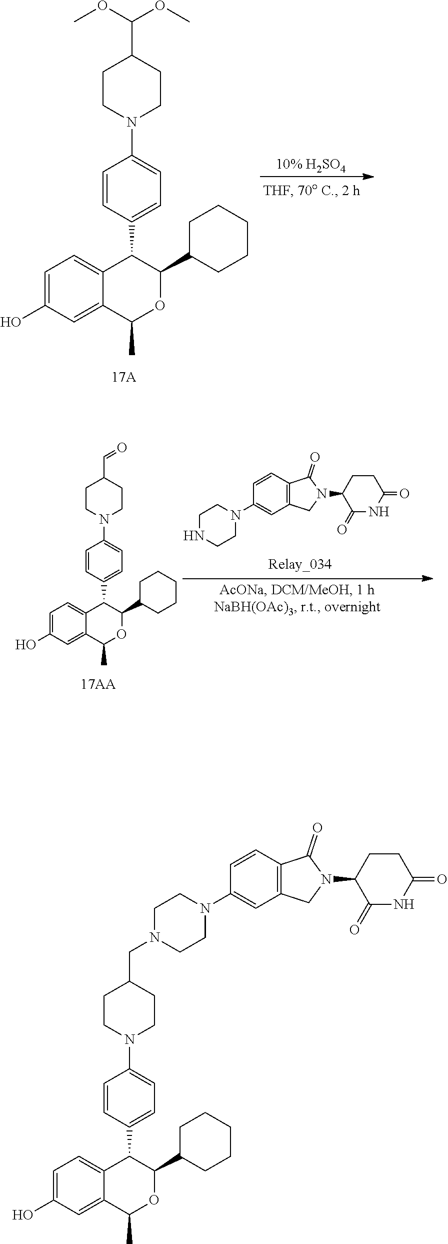 [SMILES: COC(OC)C1CCN(c2ccc([C@H]3c4ccc(O)cc4[C@H](C)O[C@@H]3C3CCCCC3)cc2)CC1 ; C[C@@H]1O[C@H](C2CCCCC2)[C@@H](c2ccc(N3CCC(C=O)CC3)cc2)c2ccc(O)cc21 ; O=C1CC[C@H](N2Cc3cc(N4CCNCC4)ccc3C2=O)C(=O)N1 ; C[C@@H]1O[C@H](C2CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21]

Step 1:17 was further separated by SFC (condition: column: DAICEL CHIRALPAK AD (250 mm*30 mm, 10 um)); mobile phase: CO₂-EtOH (0.1% NH₃H₂O); B %: 30%-30%) to give (1S,3R,4S)-3-cyclohexyl-4-(4-(4-(dimethoxymethyl)piperidin-1-yl)phenyl)-1-methylisochroman-7-ol (12 mg, 25 μmol, 40% yield) and (1R,3S,4R)-3-cyclohexyl-4-(4-(4-(dimethoxymethyl)piperidin-1-yl)phenyl)-1-methylisochroman-7-ol (13 mg, 27.1 μmol, 43.3% yield) and as a white solid.

Step 2: A solution of 1-(4-((1S,3R,4S)-3-cyclohexyl-7-hydroxy-1-methylisochroman-4-yl)phenyl)piperidine-4-carbaldehyde (9 mg, 0.02 mmol, 100%) in 10% H₂SO₄ (2 mL) and THE (2 mL) was stirred at 70° C. for 2 hour. The reaction was adjust to pH=7 with saturated aqueous NaHCO₃, then 50 mL water was added, and the mixture was extracted with dichloromethane (100 mL*2). The combined organic layers were washed with brine (20 mL), dried over anhydrous Na₂SO₄, filtered and concentrated to dryness in vacuo to give a yellow oil. The yellow oil was concentrated in vacuo to give 1-(4-((1S, 3R, 4S)-3-cyclohexyl-7-hydroxy-1-methylisochroman-4-yl)phenyl)piperidine-4-carbaldehyde (9 mg, 0.02 mmol, 100% yield) as a yellow solid. LC-MS (ESI⁺) m/z: 434.3 (M+H)⁺.

Step 3: To a solution of 1-(4-((1S,3R,4S)-3-cyclohexyl-7-hydroxy-1-methylisochroman-4-yl)phenyl)piperidine-4-carbaldehyde (9 mg, 100% purity, 1 Eq, 0.02 mmol), (S)-3-(1-oxo-5-(piperazin-1-yl)isoindolin-2-yl)piperidine-2,6-dione HCl salt (8 mg, 1.1 Eq, 0.02 mmol) in DCM (1 mL), MeOH (1 mL) was added sodium acetate (5 mg, 3 Eq, 0.06 mmol) was stirred at 25° C. for 1 h, Then sodium triacetoxyhydroborate (9 mg, 2 Eq, 0.04 mmol) and acetic acid (4 mg, 4 μL, 3 Eq, 0.06 mmol) was added. The mixture was stirred at 25° C. for 15 hour. The reaction mixture was concentrated under reduced pressure to remove solvent. The residue was purified by prep-HPLC (column: Welch Xtimate C18 150*30 mm*5 um phase: [water (FA)-ACN]; B %: 15%-55%, 9 min to give (S)-3-(5-(4-((1-(4-((1S,3R,4S)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione (2.1 mg, 2.6 μmol, 10% yield, 91.9% purity) as a white solid.

LCMS: calc. for C₄₅H₅₅N₅O₅: 745.42, found: [M+H]⁺746.5.

HPLC: 91.9% purity at 220 nm.

¹H NMR (400 MHz, DMSO-d₆) δ 10.94-11.00 (m, 1H) 9.16-9.24 (m, 1H) 8.15 (s, 0.310H) 7.53 (d, J=8.78 Hz, 1H) 7.03-7.13 (m, 4H) 6.70-6.81 (m, 3H) 6.47-6.56 (m, 2H) 5.06 (dd, J=13.30, 5.27 Hz, 1H) 4.72 (q, J=6.19 Hz, 1H) 4.28-4.38 (m, 1H) 4.18-4.25 (m, 1H) 3.75 (br s, 1H) 3.56-3.66 (m, 2H) 3.25-3.34 (m, 7H) 2.82-3.05 (m, 1H) 2.54-2.63 (m, 4H) 2.30-2.48 (m, 2H) 2.18-2.27 (m, 2H) 1.95-2.06 (m, 2H) 1.80 (br d, J=11.04 Hz, 3H) 1.58-1.70 (m, 3H) 1.51-1.57 (m, 4H) 1.15-1.22 (m, 2H) 1.05-1.12 (m, 1H) 0.86-1.03 (m, 5H).

The absolute stereochemistry was arbitrarily assigned.

### Example 114. Preparation of (I-389) (R)-3-(4-(2-((1-(4-((3S,4R)-7-hydroxy-3-phenylisochroman-4-yl)phenyl)piperidin-4-yl)methyl)-2,8-diazaspiro[4.5]decan-8-yl)phenyl)piperidine-2,6-dione and (I-390) (S)-3-(4-(2-((1-(4-((3S,4R)-7-hydroxy-3-phenylisochroman-4-yl)phenyl)piperidin-4-yl)methyl)-2,8-diazaspiro[4.5]decan-8-yl)phenyl)piperidine-2,6-dione

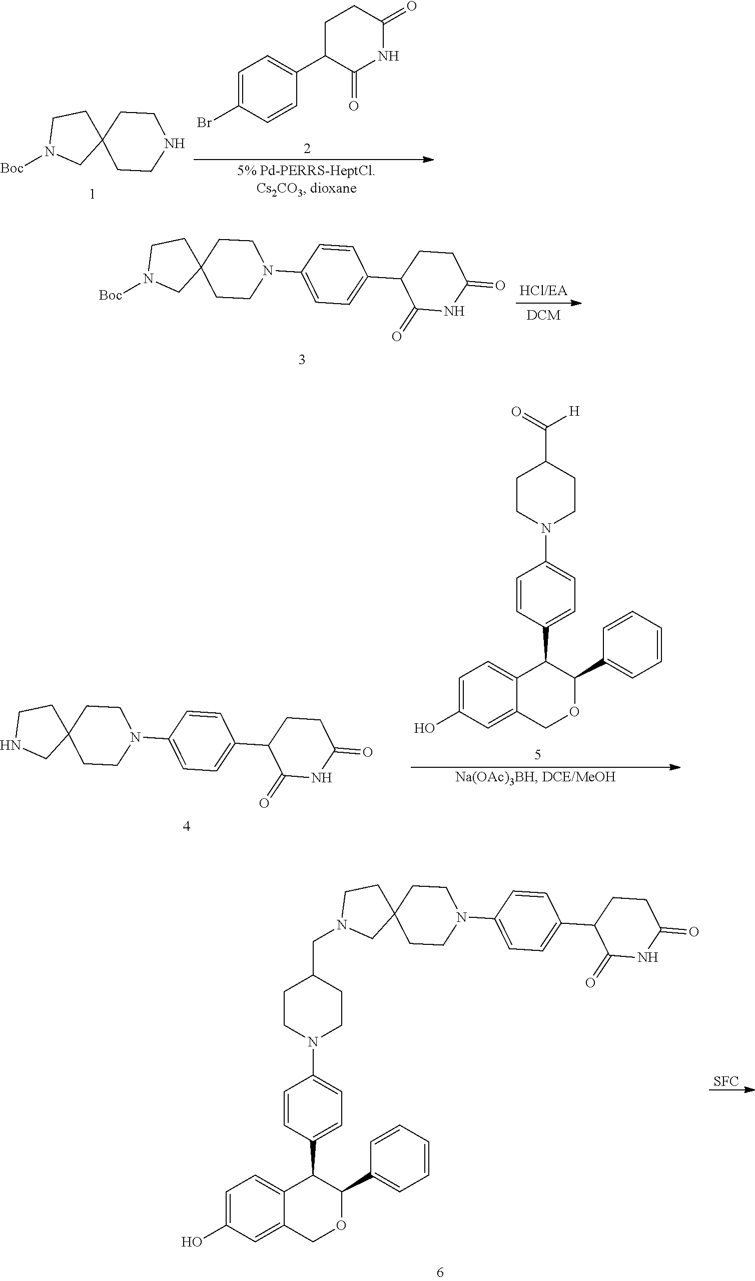

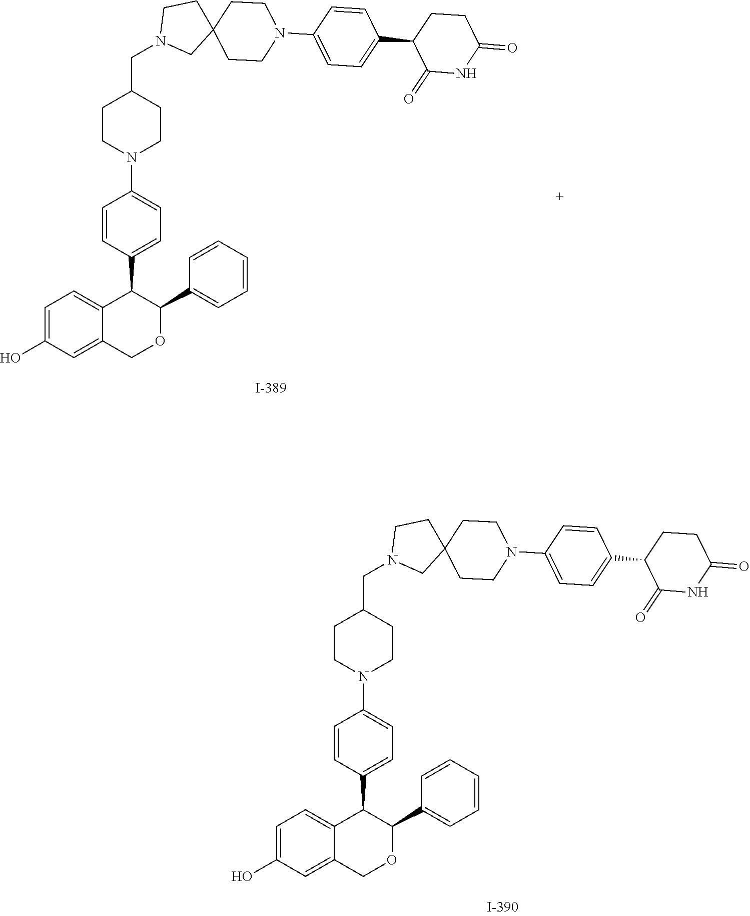 [SMILES: O=C1CC[C@H](c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1 ; "+" ; O=C1CC[C@@H](c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1]

Step 1: To a solution of compound 1 (900.00 mg, 3.74 mmol, 2 eq.) in dioxane (15 mL) was added compound 2 (502 mg, 1.87 mmol, 1 eq.), Cs₂CO₃ (1.22 g, 3.74 mmol, 2 eq) and 5% Pd-PEPPSI-IHeptCI (182.00 mg, 187 μmol, 0.1 eq). The mixture was stirred at 90° C. under N₂ for 2 hr. LCMS showed the desired ms was found. The mixture was concentrated to give a residue, which was purified by flash silica gel chromatography eluting with EtOAc in PE from 0% to 100% to give compound 3 (440.00 mg, 83.80% purity) as a yellow solid.

LC-MS (ESI⁺) m/z: 428.2 (M+H+).

Step 2: To a solution of compound 3 (440.00 mg, 1.03 mmol, 1 eq.) in DCM (20 mL) was added HCl/EA (2.57 mL, 10.3 mmol, 10 eq.). The mixture was stirred at 20° C. for 1 hr. LCMS showed the desired ms was found. After completion, the mixture was concentrated under reduced pressure to give compound 4 (450.00 mg, crude) as a yellow solid without purification.

LC-MS (ESI⁺) m/z: 328.1 (M+H)⁺.

Step 3: To a solution of compound 4 (215.00 mg, 655.00 μmol, 1 eq.) in DCE (10 mL) and MeOH (5 mL) was added compound 5 (135.00 mg, 327.00 μmol, 0.5 eq.) and NaBH(OAc)₃ (1.66 g, 7.86 mmol, 12 eq.). The mixture was stirred at 20° C. for 1 hr. The mixture was purified by Prep-HPLC (Column: Boston Green ODS 150*30 mm*5 um; Condition water (FA)-CAN, B %: 10%-40%, 12 min) to give compound 6 (110.00 mg, 20.5% yield, 88.34% purity) as a yellow solid.

LC-MS (ESI⁺) m/z: 723.4 (M+H)⁺.

Step 4: Compound 6 (110.00 mg, 152.00 μmol, 1 eq.) was separated by chiral SFC (column; DAICEL CHIRALPAK IE 250 mm*30 mm, 10 um); mobile phase: [MeOH-ACN]; B %: 50%-50%; Flow Rate: 80 ml/min; peak 1:2.980 min; peak 2:6.878 min) to yield peak 1 and peak 2.

Peak 1 was concentrated under reduced pressure to yield compound I-389 (34.1 mg, 47.0 μmol, 31.0% yield, 100% purity) as a white solid.

LC-MS (ESI⁺) m/z: 725.4 (M+H)⁺.

LCMS: calc. C₄₆H₅₂N₄O₄ for: 724.95, found: [M+H]⁺725.4.

HPLC: 100% purity at 220 nm.

¹H NMR: ¹H NMR (400 MHZ, DMSO-d₆) δ=10.76 (s, 1H), 9.28 (s, 1H), 7.15-7.11 (m, 2H), 7.08 (br d, J=6.8 Hz, 1H), 7.06-7.01 (m, 4H), 6.87 (d, J=8.8 Hz, 2H), 6.77 (d, J=8.1 Hz, 1H), 6.61-6.58 (m, 2H), 6.56-6.52 (m, 4H), 5.09-4.99 (m, 2H), 4.90 (br d, J=15.3 Hz, 1H), 4.02 (d, J=3.1 Hz, 1H), 3.73-3.68 (m, 1H), 3.47 (br d, J=11.5 Hz, 2H), 3.13-3.05 (m, 4H), 2.64-2.57 (m, 1H), 2.47-2.42 (m, 4H), 2.32 (s, 2H), 2.21 (br d, J=7.0 Hz, 2H), 2.13-2.07 (m, 1H), 2.02-1.97 (m, 1H), 1.77-1.71 (m, 2H), 1.61-1.54 (m, 6H), 1.50-1.45 (m, 1H), 1.24-1.05 (m, 3H).

Peak 2 was concentrated under reduced pressure to yield compound I-390 (41.3 mg, 57.0 μmol, 37.5% yield, 100% purity) as a white solid.

LC-MS (ESI⁺) m/z: 725.4 (M+H)⁺.

LCMS: calc. C₄₆H₅₂N₄O₄ for: 724.95, found: [M+H]⁺725.4.

HPLC: 100% purity at 220 nm.

¹H NMR: ¹H NMR (400 MHZ, DMSO-d₆) δ=10.79-10.72 (m, 1H), 9.35-9.26 (m, 1H), 7.15-7.11 (m, 2H), 7.09 (br d, J=6.8 Hz, 1H), 7.07-7.01 (m, 4H), 6.87 (d, J=8.7 Hz, 2H), 6.77 (d, J=7.9 Hz, 1H), 6.62-6.58 (m, 2H), 6.57-6.52 (m, 4H), 5.09-4.99 (m, 2H), 4.90 (br d, J=15.3 Hz, 1H), 4.02 (d, J=2.7 Hz, 1H), 3.73-3.68 (m, 1H), 3.47 (br d, J=12.5 Hz, 2H), 3.15-3.05 (m, 4H), 2.65-2.58 (m, 1H), 2.48-2.41 (m, 4H), 2.31 (s, 2H), 2.21 (br d, J=7.1 Hz, 2H), 2.13-2.06 (m, 1H), 2.02-1.96 (m, 1H), 1.77-1.70 (m, 2H), 1.61-1.53 (m, 6H), 1.51-1.45 (m, 1H), 1.24-0.97 (m, 3H).

### Example 115. Preparation of (I-357) (S)-3-(5-(4-((1-(4-((3S,4R)-3-(5-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione and (I-358) (S)-3-(5-(4-((1-(4-((3R,4S)-3-(5-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione

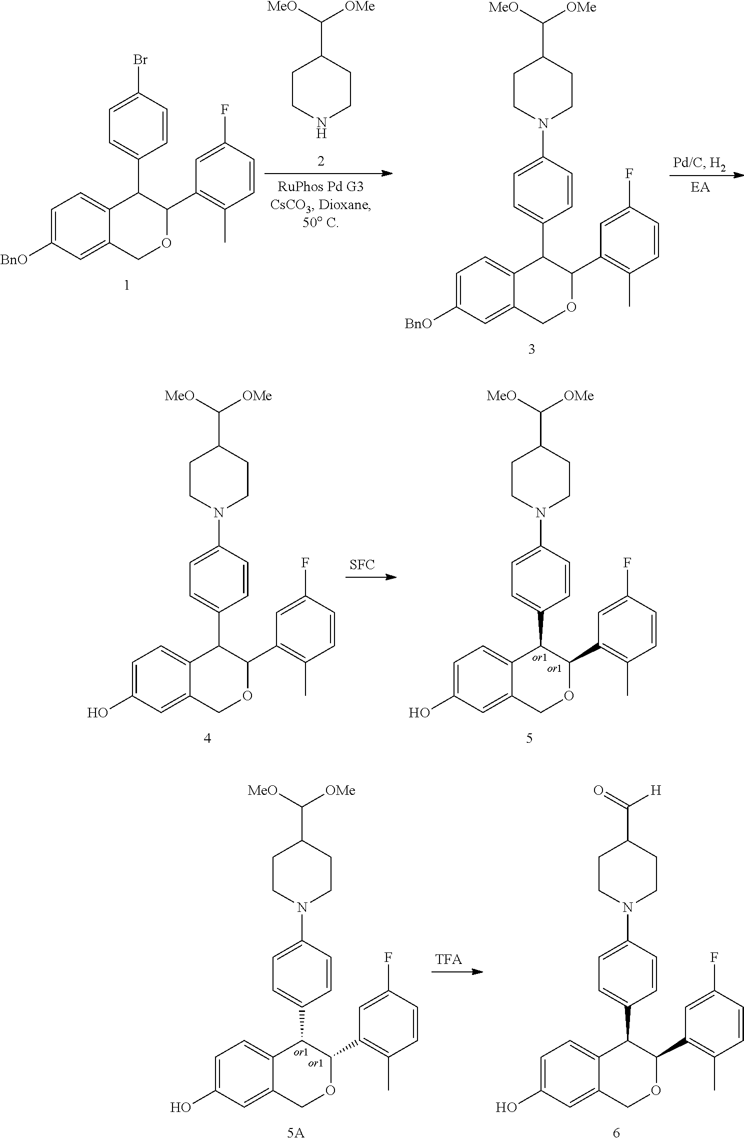

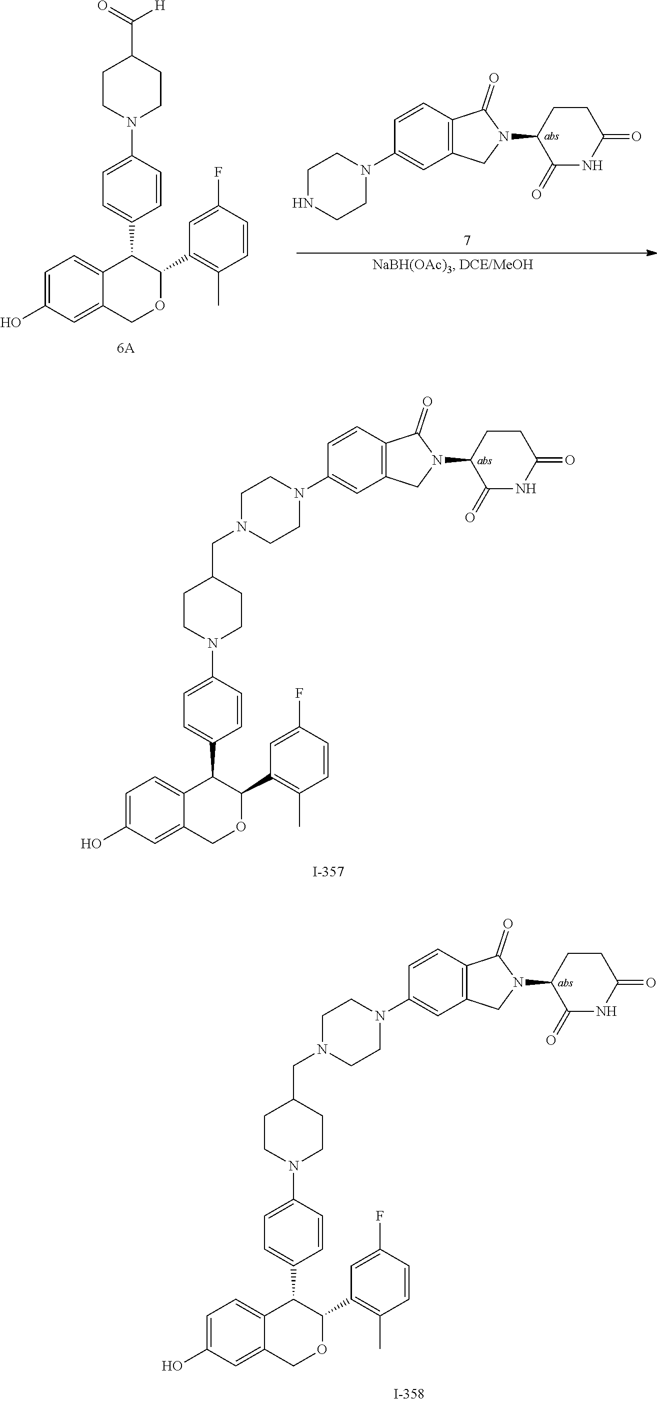 [SMILES: Cc1ccc(F)cc1[C@@H]1OCc2cc(O)ccc2[C@@H]1c1ccc(N2CCC(C=O)CC2)cc1 ; O=C1CC[C@H](N2Cc3cc(N4CCNCC4)ccc3C2=O)C(=O)N1 ; Cc1ccc(F)cc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1 ; Cc1ccc(F)cc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1]

Step 1: To a solution of compound 1 (90 mg, 178.78 μmol, 1 eq.), compound 2 (31.31 mg, 196.66 μmol, 1.1 eq.), Cs₂CO₃ (116.50 mg, 357.57 μmol, 2 eq.) and RuPhos Pd G3 (14.95 mg, 17.88 μmol, 0.1 eq.) in Dioxane (2 mL) was bubbled with N₂ for 1 min. Then the mixture was stirred at 90° C. for 5 hours. LCMS showed the starting material was consumed completely and a major peak with desired MS was detected. The reaction mixture was concentrated to get a residue. The residue was purified by prep-TLC (SiO₂, Petroleum ether/Ethyl acetate==3/1) to give compound 2 (80 mg, 53.85% yield) as a white solid.

LC-MS (ESI⁺) m/z: 582.3 (M+H)⁺.

¹H NMR (400 MHZ, DMSO-d₆) δ=7.49-7.31 (m, 5H), 7.15-7.07 (m, 1H), 6.95-6.77 (m, 4H), 6.61-6.48 (m, 4H), 6.36-6.29 (m, 1H), 5.17-5.04 (m, 4H), 4.18-3.95 (m, 3H), 3.56-3.47 (m, 2H), 3.24 (s, 6H), 2.48-2.40 (m, 2H), 2.38-2.31 (m, 3H), 1.70-1.57 (m, 3H), 1.31-1.20 (m, 2H)

Step 2: To a solution of compound 3 (80 mg, 137.52 μmol, 1 eq.) in EA (10 mL) was added Pd/C (146.35 mg, 137.52 μmol, 10% purity, 1 eq.) under N₂ atmosphere. The suspension was degassed and purged with H₂ for 3 times. The mixture was stirred under H₂ (15 Psi) at 20° C. for 3 hr. LCMS showed the starting material was consumed completely and a major peak with desired MS was detected. The resulting solution was filtrated and concentrated to give a crude. The residue was purified by prep-HPLC (column: Boston Green ODS 150*30 mm*5 um; mobile phase: [water (FA)-ACN]; gradient: 17%-47% B over 12 min) to give compound 4 (50 mg, 62.86% yield) as a white solid.

LC-MS (ESI⁺) m/z: 492.2 (M+H)⁺.

Step 3: The compound 4 (50 mg, 101.71 μmol, 1 eq.) was purified by prep-SFC (column: DAICEL CHIRALPAK AD (250 mm*30 mm, 10 um); mobile phase: [CO₂-EtOH (0.1% NH₃H₂O)]; B %: 30% %, isocratic elution mode) to give compound 5 (15 mg, 30.00% yield) as a white solid and compound 5A (13 mg, 26.00% yield) as a white solid.

Compound 5: LC-MS (ESI⁺) m/z: 492.2 (M+H)⁺.

Compound 5A: LC-MS (ESI⁺) m/z: 492.2 (M+H)⁺.

Step 4: To a solution of compound 5 (15 mg, 31 μmol, 1 eq.) in DCM (3 mL) was added TFA (1 mL) at 20° C. The mixture was stirred at 20° C. for 2 hour. LCMS showed the starting material was consumed completely and a major peak with desired MS was detected. Water (10 ml) and aq. NaHCO₃ (10×2 mL) was added and the mixture was extracted with DCM (15 ml×3). The organics were washed with brine (15 ml), dried over Na₂SO₄, filtered and concentrated to give compound 6 (13 mg, 93% yield) as a yellow solid.

LC-MS (ESI⁺) m/z: 446.3 (M+H)⁺.

Step 5: To a solution of compound 5A (13 mg, 26 μmol, 1 eq.) in DCM (3 mL) was added TFA (1 mL) at 20° C. The mixture was stirred at 20° C. for 2 hour. LCMS showed the starting material was consumed completely and a major peak with desired MS was detected. Water (10 ml) and aq. NaHCO₃ (10×2 mL) was added and the mixture was extracted with DCM (15 ml×3). The organics were washed with brine (15 ml), dried over Na₂SO₄, filtered and concentrated to give compound 6A (10 mg, 83% yield) as a yellow solid.

LC-MS (ESI⁺) m/z: 446.3 (M+H)⁺.

Step 6: To a solution of compound 6 (13 mg, 29 μmol, 1 eq.) and compound 7 (11 mg, 32 μmol, 1.1 eq.) in DCE (4 mL) and MeOH (1 mL) was added NaBH(OAc)₃ (31 mg, 0.15 mmol, 5 eq.) at 20° C. Then the mixture was stirred at 20° C. for 2 hours. LCMS showed the starting material was consumed completely and a major peak with desired MS was detected. The reaction mixture was concentrated to get the crude, which was purified by prep-HPLC (column: Boston Green ODS 150*30 mm*5 um; mobile phase: water (FA)-ACN; gradient: 15%-45% B over 12 min) to give (S)-3-(5-(4-((1-(4-((3S,4R)-3-(5-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione (8.4 mg, 38% yield) as a white solid.

LC-MS (ESI⁺) m/z: 758.4 (M+H)⁺.

LCMS: calc. for C₄₅H₄₈FN₅O₅: 757.36, found: [M+H]⁺758.4.

HPLC: 100.00% purity at 220 nm

¹H NMR (400 MHZ, DMSO-d₆) δ=10.69 (s, 1H), 9.05 (br s, 1H), 7.64-7.57 (m, 1H), 7.38-7.21 (m, 1H), 7.13-7.09 (m, 2H), 6.82-6.76 (m, 2H), 6.62-6.54 (m, 6H), 6.42-6.34 (m, 1H), 5.09-4.93 (m, 4H), 4.39-4.25 (m, 2H), 4.04 (d, J=2.9 Hz, 2H), 3.53 (br d, J=12.4 Hz, 3H), 3.37-3.19 (m, 4H), 2.95-2.82 (m, 2H), 2.71-2.55 (m, 4H), 2.49-2.26 (m, 6H), 2.04-1.78 (m, 4H), 1.35-1.20 (m, 2H)

Step 7: To a solution compound 6A (10 mg, 22 μmol, 1 eq.) and compound 7A (8.1 mg, 25 μmol, 1.1 eq.) in DCE (4 mL) and MeOH (1 mL) was added NaBH(OAc)₃ (24 mg, 0.11 mmol, 5 eq.) at 20° C. Then the mixture was stirred at 20° C. for 2 hours. LCMS showed the starting material was consumed completely and a major peak with desired MS was detected. The mixture was concentrated to get the crude, which was purified by prep-HPLC (column: Boston Green ODS 150*30 mm*5 um; mobile phase: water (FA)-ACN; gradient: 15%-45% B over 12 min) to give (S)-3-(5-(4-((1-(4-((3R,4S)-3-(5-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione (8.3 mg, 49% yield) as a white solid.

LC-MS (ESI⁺) m/z: 758.4 (M+H)⁺.

LCMS: calc. for C₄₅H₄₈FN₅O₅: 757.36, found: [M+H]⁺758.4.

HPLC: 100.00% purity at 220 nm

¹H NMR (400 MHZ, DMSO-d₆) δ=10.69 (br s, 1H), 9.05 (br s, 1H), 7.64-7.56 (m, 1H), 7.37-7.21 (m, 1H), 7.12-7.09 (m, 2H), 6.82-6.75 (m, 2H), 6.61-6.53 (m, 6H), 6.42-6.33 (m, 1H), 5.14-4.89 (m, 4H), 4.40-4.20 (m, 2H), 4.04 (d, J=2.5 Hz, 2H), 3.60-3.46 (m, 3H), 3.37-3.15 (m, 4H), 2.97-2.82 (m, 2H), 2.71-2.55 (m, 4H), 2.35 (s, 5H), 2.03-1.75 (m, 4H), 1.34-1.16 (m, 2H).

### Example 116. Preparation of (I-387) (S)-3-(5-(4-((1-(4-((3S,4R)-7-hydroxy-3-phenylisochroman-4-yl)phenyl)piperidin-4-yl)methyl)piperazin-1-yl)-3-methyl-2-oxo-2,3-dihydro-1H-benzo[d]imidazol-1-yl)piperidine-2,6-dione and (I-388) (R)-3-(5-(4-((1-(4-((3S,4R)-7-hydroxy-3-phenylisochroman-4-yl)phenyl)piperidin-4-yl)methyl)piperazin-1-yl)-3-methyl-2-oxo-2,3-dihydro-1H-benzo[d]imidazol-1-yl)piperidine-2,6-dione

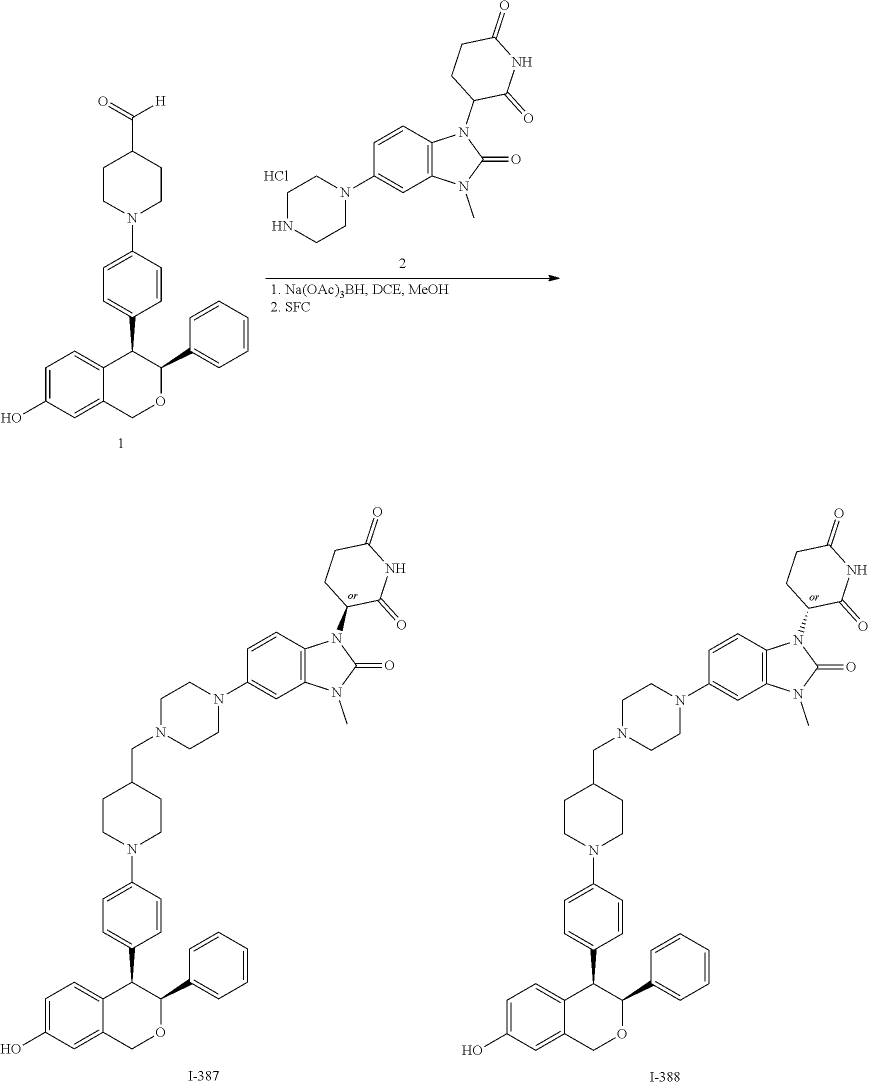 [SMILES: O=CC1CCN(c2ccc([C@@H]3c4ccc(O)cc4CO[C@@H]3c3ccccc3)cc2)CC1 ; Cl ; Cn1c(=O)n(C2CCC(=O)NC2=O)c2ccc(N3CCNCC3)cc21 ; Cn1c(=O)n([C@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21 ; Cn1c(=O)n([C@@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21]

To a solution of compound 1 (140 mg, 1 eq, 339 μmol) in DCE (10 mL) and MeOH (1 mL) was added compound 2 (116 mg, 1 eq, 339 μmol) and stirred at 20° C. for 10 min. Then sodium triacetoxyhydroborate (215 mg, 3 eq, 1.02 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 45%-75% B over 11 min), followed by lyophilization to yield a residue which was separated by chiral SFC (column: DAICEL CHIRALPAK ID (250 mm*30 mm, 10 um); mobile phase: [MeOH-ACN] B: 50%, isocratic elution mode, peak 1:2.324 min, peak 2:3.447 min) to yield Peak 1 and Peak 2.

Peak 1 was concentrated under reduced pressure to give the product of 1-387 (25.5 mg, 36% yield, SFC: R_{f}=2.303) as a white solid. LC-MS (ESI⁺) m/z: 741.3 (M+H)⁺,

LCMS: calc. for C₄₄H₄₈N₆O₅:740.91, found: [M+H]⁺741.3.

HPLC: 100.0% purity at 254 nm.

¹HNMR (500 MHz, DMSO-d₆): $11.05 (s, 1H), 9.28 (s, 1H), 7.20-7.01 (m, 5H), 6.93 (d, J=8.5 Hz, 1H), 6.83 (d, J=2.1 Hz, 1H), 6.78 (d, J=8.1 Hz, 1H), 6.66-6.58 (m, 3H), 6.58-6.50 (m, 4H), 5.28 (dd, J=5.4, 12.9 Hz, 1H), 5.07 (d, J=15.6 Hz, 1H), 5.00 (d, J=3.1 Hz, 1H), 4.90 (d, J=15.3 Hz, 1H), 4.03 (d, J=2.7 Hz, 1H), 3.49 (d, J=11.7 Hz, 2H), 3.30-3.28 (m, 3H), 3.07 (s, 4H), 2.93-2.81 (m, 1H), 2.76-2.56 (m, 2H), 2.51 (s, 2H), 2.46 (d, J=5.5 Hz, 4H), 2.18 (d, J=7.2 Hz, 2H), 2.06-1.93 (m, 1H), 1.74 (d, J=11.9 Hz, 2H), 1.61 (dd, J=3.7, 7.2 Hz, 1H), 1.21-1.05 (m, 2H)

Peak 2 was concentrated under reduced pressure to yield a product I-388 (27.9 mg, 40% yield, SFC: Rt=3.270) as a white solid. LC-MS (ESI⁺) m/z: 741.3 (M+H)⁺.

LCMS: calc. for C₄₄H₄₈N₆O₅: 740.91, found: [M+H]⁺741.3.

HPLC: 99.53% purity at 254 nm.

¹HNMR (500 MHz, DMSO-d₆): δ 10.98 (s, 1H), 9.28 (s, 1H), 7.18-7.11 (m, 2H), 7.10-7.04 (m, 3H), 6.93 (d, J=8.5 Hz, 1H), 6.83 (d, J=2.1 Hz, 1H), 6.78 (d, J=8.1 Hz, 1H), 6.65-6.58 (m, 3H), 6.58-6.52 (m, 4H), 5.28 (dd, J=5.4, 12.9 Hz, 1H), 5.07 (d, J=15.4 Hz, 1H), 5.00 (d, J=3.1 Hz, 1H), 4.90 (d, J=15.3 Hz, 1H), 4.03 (d, J=2.9 Hz, 1H), 3.49 (d, J=11.9 Hz, 2H), 3.29 (s, 3H), 3.07 (s, 4H), 2.95-2.83 (m, 1H), 2.74-2.57 (m, 2H), 2.74-2.57 (m, 1H), 2.74-2.57 (m, 1H), 2.54 (s, 2H), 2.46 (d, J=5.5 Hz, 4H), 2.18 (d, J=7.2 Hz, 2H), 2.02-1.91 (m, 1H), 1.74 (d, J=11.6 Hz, 2H), 1.61 (td, J=3.5, 7.0 Hz, 1H), 1.14 (q, J=11.7 Hz, 2H).

### Example 117. Preparation of (I-251) (S)-3-(5-(4-((1-(4-((1R,3S,4S)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione

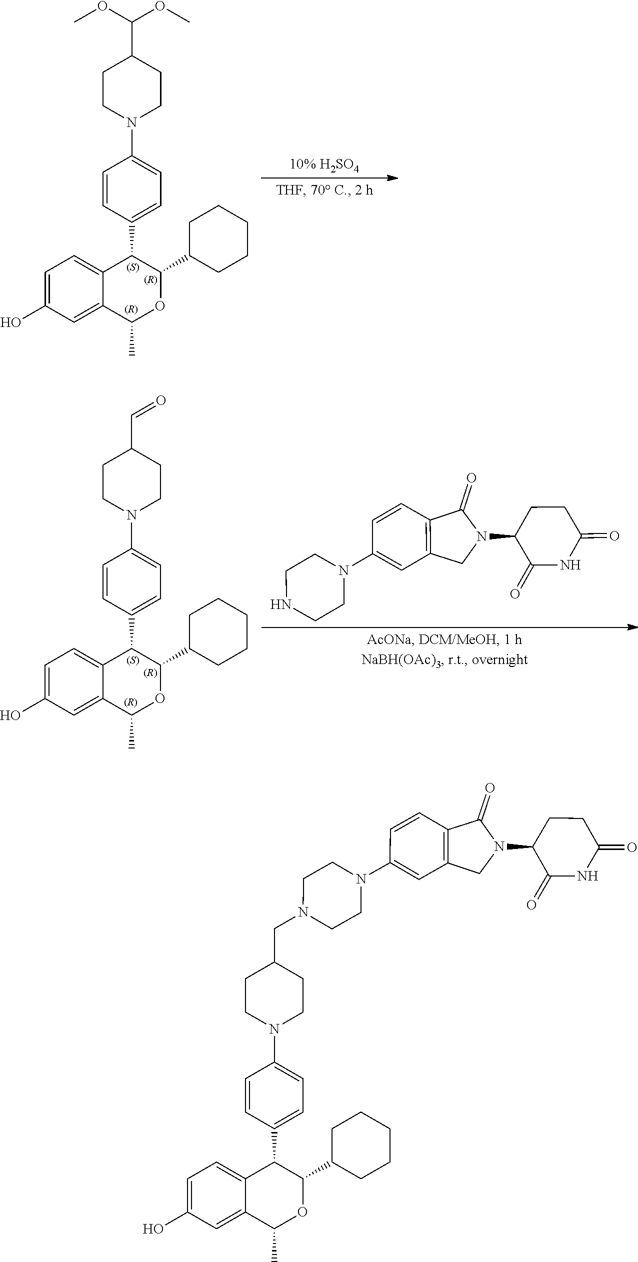

Step 1: To a solution of 1-(4-((1R,3S,4S)-3-cyclohexyl-7-hydroxy-1-methylisochroman-4-yl)phenyl)piperidine-4-carbaldehyde (17 mg, 39.2 μmol, 94% purity) in 10% H₂SO₄ (2 mL) and THF (2 mL) was stirred at 70° C. for 2 hour. The reaction was adjusted to pH=˜7 with saturated aqueous NaHCO₃, then 20 mL water was added, and the mixture was extracted with dichloromethane (20 mL*2). The combined organic layers were washed with brine (20 mL), dried over anhydrous Na₂SO₄, filtered and concentrated to dryness in vacuo to give a yellow oil. The yellow oil was concentrated in vacuo to give 1-(4-((1R, 3S, 4S)-3-cyclohexyl-7-hydroxy-1-methylisochroman-4-yl)phenyl)piperidine-4-carbaldehyde (17 mg, 39.2 μmol, 94% purity) as a yellow solid. LC-MS (ESI⁺) m/z: 452.5 (M+H₂O)⁺.

Step 2: To a solution of 1-(4-((1R,3S,4S)-3-cyclohexyl-7-hydroxy-1-methylisochroman-4-yl)phenyl)piperidine-4-carbaldehyde (17 mg, 1 Eq, 39.2 μmol), (S)-3-(1-oxo-5-(piperazin-1-yl)isoindolin-2-yl)piperidine-2,6-dione (16 mg, 1.1 Eq, 43.1 μmol, HCl salt) in DCM (2 mL), MeOH (2 mL) was added sodium acetate (10 mg, 3 Eq, 118 μmol) was stirred at 25° C. for 1 h, Then sodium triacetoxyhydroborate (17 mg, 3 Eq, 118 μmol) and acetic acid (7 mg, 7 μL, 3 Eq, 118 μmol) was added. The mixture was stirred at 25° C. for 15 hours. The reaction mixture was concentrated under reduced pressure to remove solvent. The residue was purified by prep-HPLC (column: Welch Xtimate C18 150*30 mm*5 um phase: [water (FA)-ACN]; B %: 20%-60%,9 min to give (S)-3-(5-(4-((1-(4-((1R,3S,4S)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione (13.1 mg, 17 μmol, 44% yield, 98% purity) as a white solid. LC-MS (ESI⁺) m/z: 746.5 (M+H)⁺.

LCMS: calc. for C₄₅H₅₅N₅O₅: 745.42, found: [M+H]⁺746.5.

HPLC: 98.1% purity at 220 nm.

¹H NMR (400 MHZ, DMSO-d₆) δ 10.92-11.05 (m, 1H) 9.10-9.30 (m, 1H) 8.14-8.20 (m, 0.718H) 7.52 (d, J=8.28 Hz, 1H) 7.05-7.14 (m, 4H) 6.72-6.81 (m, 3H) 6.47-6.55 (m, 2H) 4.98-5.16 (m, 1H) 4.66-4.76 (m, 1H) 4.29-4.40 (m, 1H) 4.14-4.28 (m, 1H) 3.75 (br s, 1H) 3.58-3.66 (m, 2H) 3.29 (br s, 7H) 2.85-2.91 (m, 1H) 2.54-2.63 (m, 4H) 2.29-2.45 (m, 2H) 2.22 (br d, J=6.78 Hz, 2H) 1.95-2.05 (m, 2H) 1.78-1.86 (m, 3H) 1.59-1.69 (m, 3H) 1.50-1.56 (m, 4H) 1.18-1.27 (m, 2H) 1.08-1.13 (m, 1H) 0.87-1.03 (m, 5H).

### Example 118. Preparation of (I-359) (S)-3-(5-(4-((1-(4-((3R*,4R*)-7-hydroxy-3-(4-hydroxyphenyl)isochroman-4-yl)phenyl)piperidin-4-yl)methyl)piperazin-1-yl)-1-oxoisoindolin-2-yl)piperidine-2,6-dione and (I-360) (S)-3-(5-(4-((1-(4-((3R,4S)-7-hydroxy-3-(4-hydroxyphenyl)isochroman-4-yl)phenyl)piperidin-4-yl)methyl)piperazin-1-yl)-1-oxoisoindolin-2-yl)piperidine-2,6-dione

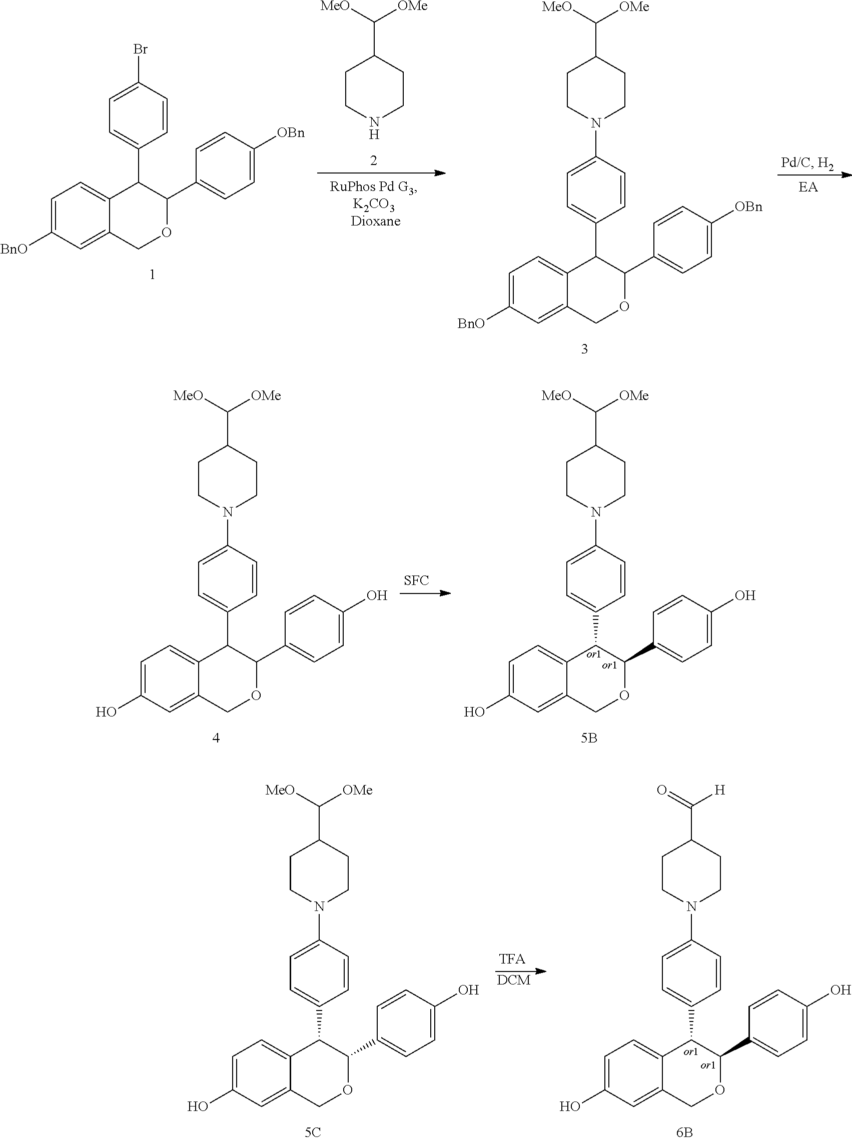

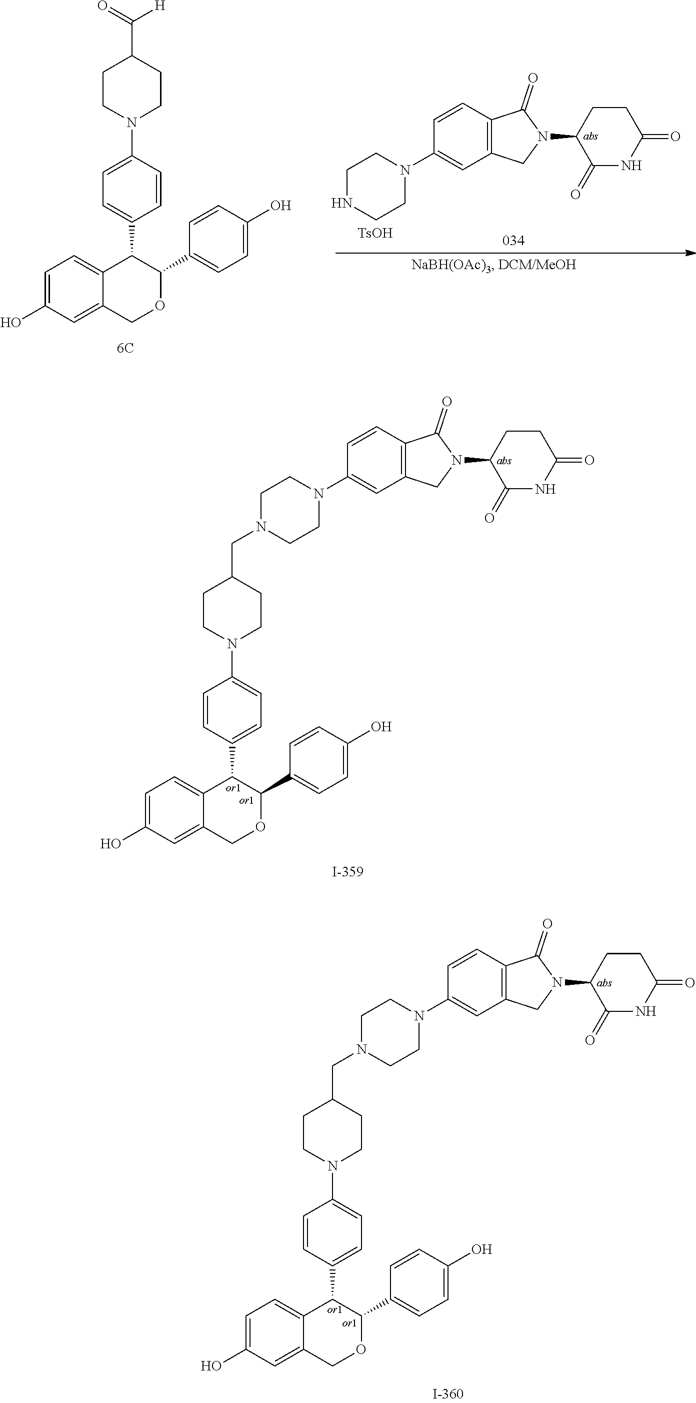

Step 1: To a solution of 1 (500 mg, 0.866 mmol, 1.0 eq.) in dioxane (2 mL) was added 2 (165.4 mg, 0.104 mmol, 1.2 eq.) and Cs₂CO₃ (846.3 mg, 0.260 mmol, 3.0 eq.) at 20° C. Then the [2-(2-aminophenyl)phenyl]-methylsulfonyloxy-palladium;dicyclohexyl-[2-(2,6-diisopropoxyphenyl)phenyl]phosphane (108.6 mg, 0.130 mmol, 0.15 eq.) was added into the mixture at 20° C. The mixture was stirred at 90° C. for 4 h under N₂. LCMS showed that the desired product was detected. The reaction mixture was filtered and concentrated under reduced pressure to get the product. The mixture was purified by Pre-TLC (PE/EA=3/1) to give 3 (300 mg, 43.78% yield) as yellow oil.

LC-MS (ESI⁺) m/z: 656.3 (M+H)⁺.

1H NMR (400 MHZ, DMSO-d₆)>=7.48-7.30 (m, 10H), 7.08 (br d, J=8.6 Hz, 1H), 6.96-6.52 (m, 10H), 5.08-4.94 (m, 5H), 4.88-4.81 (m, 1H), 4.66 (br d, J=10.1 Hz, 1H), 4.12-4.01 (m, 2H), 3.61-3.55 (m, 4H), 3.28-3.22 (m, 6H), 1.73-1.59 (m, 3H), 1.32-1.21 (m, 2H).

Step 2: A solution of 3 (280 mg, 426.95 μmol, 1.0 eq.) and Pd/C (454.36 mg, 426.95 μmol, 10% purity, 1.0 eq.) in EA (10 mL) and then degassed and purged with H₂ for 3 times, and then the mixture was stirred at 15° C. for 3 hr under H₂ atmosphere. LCMS showed that the starting material was consumed and the desired product was detected. The mixture was filtered and concentrated under vacuum to give the crude. The crude was purified by Pre-TLC (PE/EA=1/1) to give 4 (140 mg, 66.63% yield) as yellow solid.

LC-MS (ESI⁺) m/z: 476.1 (M+H)⁺.

Step 3: The 4 (152 mg, 319.61 μmol, 1.0 eq.) was further separated by SFC (condition: CO₂-EtOH (0.1% NH₃H₂O), column: DAICEL CHIRALPAK AD (250 mm×30 mm, 10 um), Begin B: 25, End B: 25). to give 5B (30 mg, 19.74% yield) as white solid and 5C (10 mg, 6.58% yield) as white solid

Only two isomers of 4 possible isomers were able to be isolated:

5B: LC-MS (ESI⁺) m/z: 476.2 (M+H)⁺.

1H NMR (500 MHz, DMSO-d₆)>=9.24 (br s, 2H), 6.94 (d, J=8.5 Hz, 2H), 6.78-6.71 (m, 4H), 6.57 (d, J=8.5 Hz, 2H), 6.50-6.45 (m, 3H), 4.95-4.74 (m, 2H), 4.54 (d, J=9.8 Hz, 1H), 4.07 (d, J=6.7 Hz, 1H), 4.02 (d, J=9.8 Hz, 1H), 3.59 (br d, J=12.2 Hz, 2H), 3.32-3.27 (m, 2H), 3.26 (s, 6H), 1.71-1.63 (m, 3H), 1.28 (br d, J=12.1 Hz, 2H).

5C: ¹H NMR (400 MHz, DMSO-d₆) δ=9.26 (s, 1H), 9.09 (s, 1H), 6.82-6.73 (m, 3H), 6.62-6.48 (m, 8H), 5.03 (d, J=15.4 Hz, 1H), 4.91-4.83 (m, 2H), 4.04 (d, J=6.7 Hz, 1H), 3.91 (d, J=2.6 Hz, 1H), 3.52 (br d, J=11.9 Hz, 2H), 3.30 (s, 2H), 3.24 (s, 6H), 1.68-1.59 (m, 3H), 1.30-1.20 (m, 2H).

Step 4: To a solution of 5B (25 mg, 1.0 Eq, 53 μmol) in DCM (1 mL) was added TFA (400 mg, 0.3 mL, 2e+1 Eq, 4 mmol) at 15° C. Then the mixture was stirred at 15° C. for 1 hour. LCMS showed that the desired product was detected. Then the mixture was filtered and concentrated under vacuum to give the 6B (20 mg, crude) as red oil.

LC-MS (ESI⁺) m/z: 446.2 (M+H+16)⁺

Step 5: To a solution of 5C (8 mg, 1.0 Eq, 20 μmol) in DCM (1 mL) was added TFA (400 mg, 0.3 mL, 2e+1 Eq, 4 mmol) at 15° C. Then the mixture was stirred at 15° C. for 1 hour. LCMS showed that the desired product was detected. Then the mixture was filtered and concentrated under vacuum to give the 6C (5 mg, crude) as red oil.

LC-MS (ESI⁺) m/z: 448.2 (M+H+18)⁺

Step 6: To a solution of 6B (30 mg, 1 Eq, 70 μmol) in DCE (1 mL) and MeOH (1 mL) was added 034 (46 mg, 2 Eq, 0.14 mmol) and Sodium triacetoxyborohydride (74 mg, 52 μL, 5 Eq, 0.35 mmol) at 15° C. Then the mixture was stirred at 15° C. for 1 hour. LCMS showed that the desired product was detected. The mixture was filtered and concentrated under vacuum to give the crude. The residue was purified by prep-HPLC (condition: Column: Phenomenex Gemini NX 150×30 mm×5 um; Condition: water(NH₄HCO₃)-ACN, Begin B 7, End B 27, Gradient Time (min) 12, 100% B Hold Time (min) 2, Flow Rate (mL/min) 25.) and to give 1-359 (7.8 mg, 15% yield) as a white solid.

LCMS: calc. for C₄₄H₄₇N₅O₆:741.89, found: [M+H]⁺742.3.

HPLC: 100.00% purity at 220 nm

¹H NMR (500 MHz, DMSO-d₆) δ=10.93 (br s, 1H), 9.24 (br s, 2H), 7.52 (d, J=8.5 Hz, 1H), 7.08-7.04 (m, 2H), 6.94 (d, J=8.5 Hz, 2H), 6.80-6.71 (m, 4H), 6.57 (d, J=8.5 Hz, 2H), 6.51-6.45 (m, 3H), 5.07-5.01 (m, 1H), 4.95-4.74 (m, 2H), 4.54 (d, J=9.8 Hz, 1H), 4.35-4.18 (m, 2H), 4.03 (d, J=9.8 Hz, 1H), 3.59 (br d, J=12.1 Hz, 2H), 3.28 (br s, 8H), 2.95-2.82 (m, 1H), 2.60-2.53 (m, 3H), 2.40-2.31 (m, 1H), 2.21 (br d, J=7.2 Hz, 2H), 2.00-1.91 (m, 1H), 1.79 (br d, J=11.9 Hz, 2H), 1.71-1.60 (m, 1H), 1.23-1.13 (m, 2H).

Step 7: To a solution of 6C (5 mg, 1 Eq, 0.01 mmol) in DCE (1 mL) and MeOH (1 mL) was added 034 (8 mg, 2 Eq, 0.02 mmol) and Sodium triacetoxyborohydride (0.01 g, 9 μL, 5 Eq, 0.06 mmol) at 15° C. Then the mixture was stirred at 15° C. for 1 hour. LCMS showed that the desired product was detected. The mixture was filtered and concentrated under vacuum to give the crude. The residue was purified by prep-HPLC (condition: Column: Phenomenex Gemini NX 150×30 mm×5 um; Condition: water(NH₄HCO₃)-ACN, Begin B 28, End B 58, Gradient Time (min) 12, 100% B Hold Time (min) 2, Flow Rate (mL/min) 25.) and to give I-360 (5.4 mg, 60% yield) as a white solid.

LCMS: calc. for C₄₄H₄₇N₅O₆: 741.89, found: [M+H]⁺742.3.

HPLC: 100.00% purity at 220 nm

¹H NMR (500 MHz, DMSO-d₆) δ=10.93 (br s, 1H), 9.32-9.01 (m, 2H), 7.51 (d, J=8.5 Hz, 1H), 7.09-7.01 (m, 2H), 6.82-6.72 (m, 3H), 6.63-6.48 (m, 8H), 5.07-4.98 (m, 2H), 4.93-4.83 (m, 2H), 4.36-4.17 (m, 2H), 3.92 (d, J=2.6 Hz, 1H), 3.51 (br d, J=12.1 Hz, 2H), 3.31-3.23 (m, 8H), 2.95-2.81 (m, 1H), 2.63-2.52 (m, 3H), 2.40-2.33 (m, 1H), 2.19 (br d, J=7.2 Hz, 2H), 2.00-1.91 (m, 1H), 1.75 (br d, J=11.3 Hz, 2H), 1.67-1.58 (m, 1H), 1.23-1.12 (m, 2H).

### Example 119. Preparation of (I-361) (S)-3-(5-(4-((1-(4-((3R,4S)-7-hydroxy-3-(3-(trifluoromethoxy)phenyl)isochroman-4-yl)phenyl)piperidin-4-yl)methyl)piperazin-1-yl)-1-oxoisoindolin-2-yl)piperidine-2,6-dione and (I-362) (S)-3-(5-(4-((1-(4-((3S,4R)-7-hydroxy-3-(3-(trifluoromethoxy)phenyl)isochroman-4-yl)phenyl)piperidin-4-yl)methyl)piperazin-1-yl)-1-oxoisoindolin-2-yl)piperidine-2,6-dione

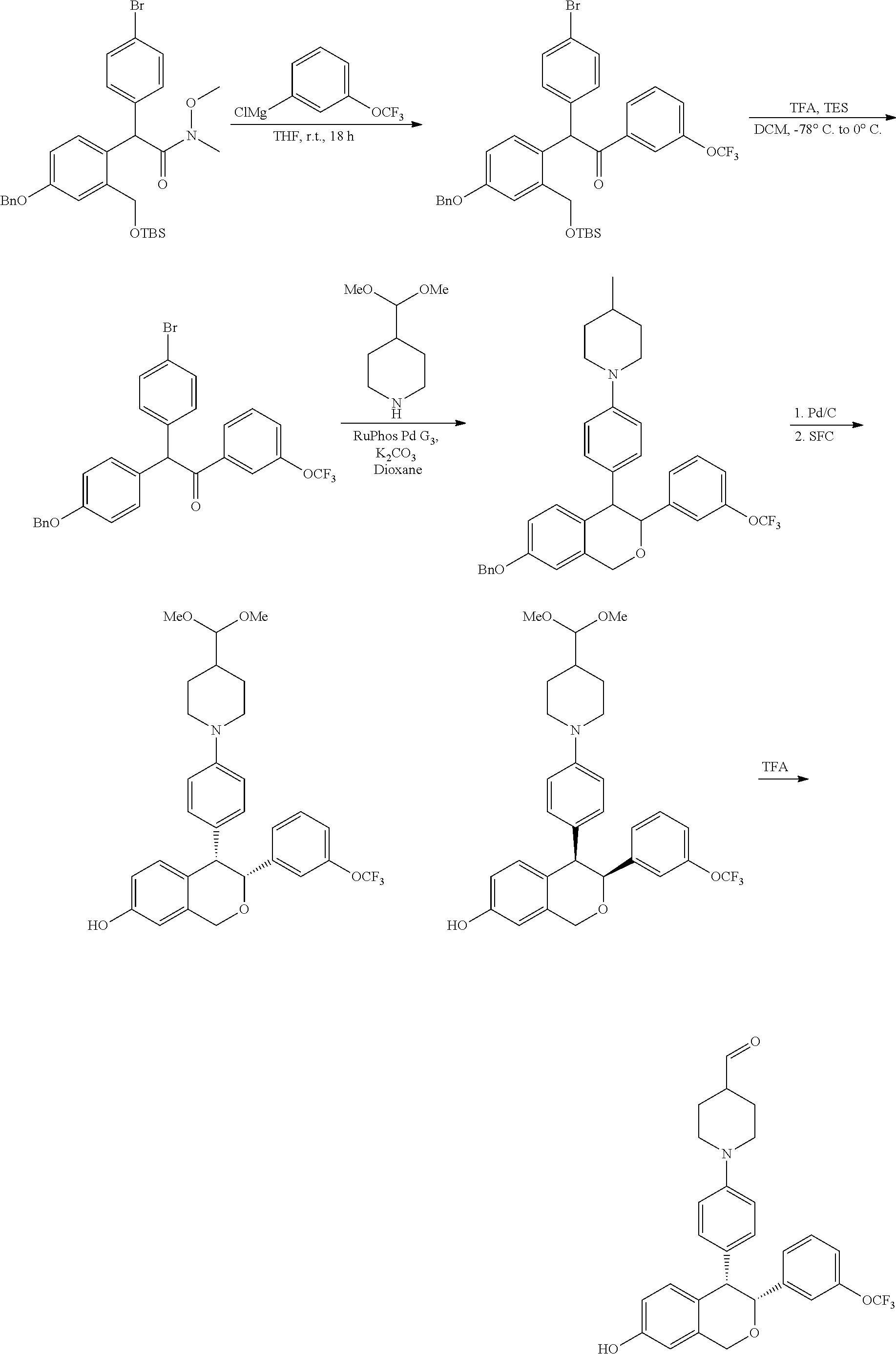

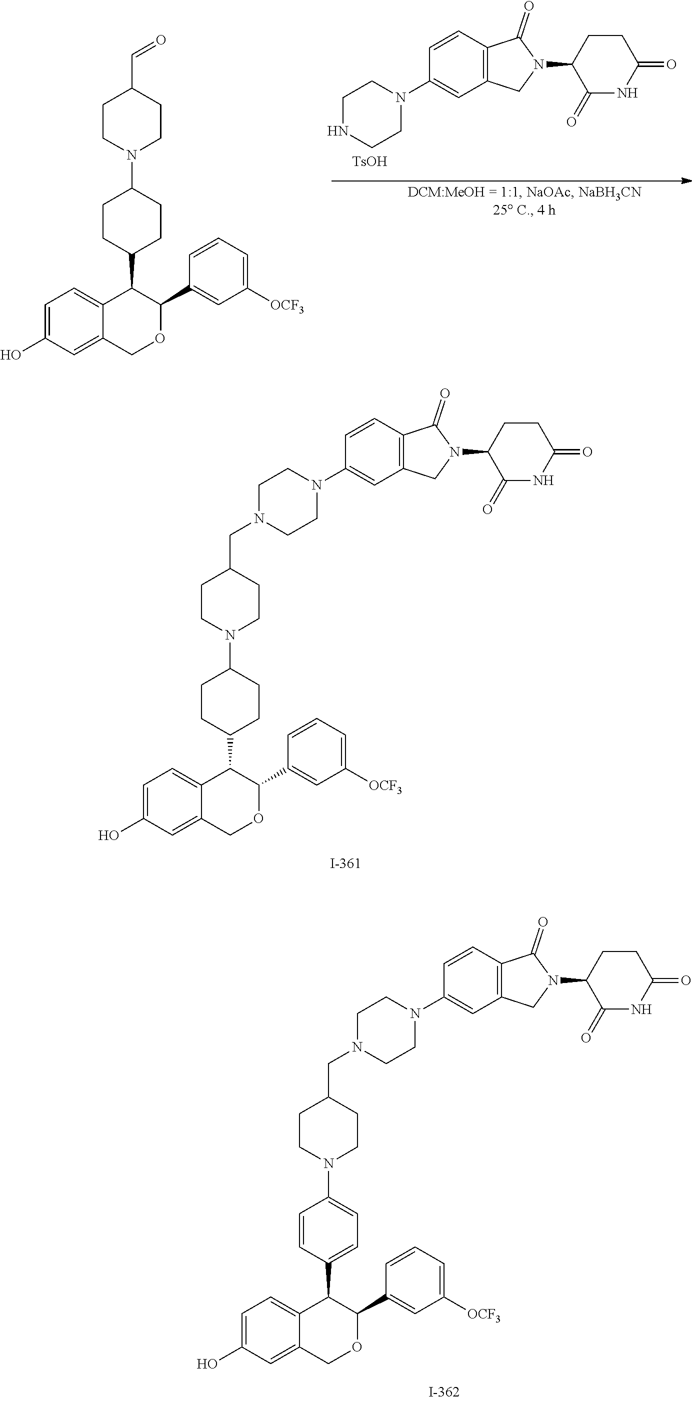

Step 1: To a solution of 1-iodo-3-(trifluoromethoxy)benzene (1.03 g, 3.59 mmol, 2.1 eq.) in THE (10 mL) was added chloro(isopropyl) magnesium (2 M, 1.88 mL, 2.2 eq.) at 0° C., after stirred 1 h was added 2-[4-benzyloxy-2-[tert-butyl(dimethyl)silyl]oxymethyl]phenyl]-2-(4-bromophenyl)-N-methoxy-N-methyl-acetamide (1 g, 1.71 mmol, 1 eq.) in THF (10 mL). The mixture was stirred at 25° C. for 12 h. The reaction mixture was quenched by addition of water (5 mL), extracted with EtOAc (10 mL×3). The combined organic layers were washed with brine (10 mL), dried over Na₂SO₄, filtered and concentrated under reduced pressure to give a residue. The residue was purified by preparative TLC (PE/EtOAc=5/1, TLC: PE/EtOAc=5/1, R_{f}=0.79) to yield 2-[4-benzyloxy-2-[tert-butyl(dimethyl)silyl]oxymethyl]phenyl]-2-(4-bromophenyl)-1-[3-(trifluoromethoxy)phenyl]ethanone (690 mg, 945.98 μmol, 55.30% yield, 94% purity) as colourless oil. LC-MS (ESI⁺) m/z: 687.1 (M+H)⁺.

¹H NMR (400 MHz, CDCl₃) δ=7.94 (d, J=7.6 Hz, 1H), 7.84 (s, 1H), 7.47-7.35 (m, 9H), 7.06-7.00 (m, 3H), 6.96 (d, J=8.5 Hz, 1H), 6.83 (dd, J=2.7, 8.5 Hz, 1H), 6.31 (s, 1H), 5.04 (s, 2H), 4.71 (d, J=12.3 Hz, 1H), 4.48 (d, J=12.4 Hz, 1H), 0.92 (s, 9H), 0.08 (d, J=2.3 Hz, 6H)

Step 2: To a solution of 2-[4-benzyloxy-2-[tert-butyl(dimethyl)silyl]oxymethyl]phenyl]-2-(4-bromophenyl)-1-[3-(trifluoromethoxy)phenyl]ethanone (690 mg, 945.98 μmol, 1 eq.) and triethylsilane (164.99 mg, 1.42 mmol, 226.64 μL, 1.5 eq.) in DCM (8 mL) was added trimethylsilyl trifluoromethanesulfonate (315.38 mg, 1.42 mmol, 256.40 μL, 1.5 eq.) at −78° C. The mixture was stirred at −78° C. for 30 min. The reaction mixture was quenched by adding saturation solution of NaHCO₃ (10 mL), extracted with DCM (10 mL×3). The combined organic layers were washed with brine (10 mL), dried over Na₂SO₄, filtered and concentrated under reduced pressure to give a residue. The residue was purified by preparative TLC (PE/EtOAc=10/1, TLC: PE/EtOAc=10/1, R_{f}=0.75) to yield 7-benzyloxy-4-(4-bromophenyl)-3-[3-(trifluoromethoxy)phenyl]isochromane (230 mg, 400.88 μmol, 42.38% yield, 96.8% purity) as a white solid. LC-MS (ESI+) m/z: 555.0 (M+H)+.

¹H NMR (400 MHZ, CDCl₃) δ=7.48-7.33 (m, 5H), 7.25-7.19 (m, 1H), 7.15 (d, J=8.3 Hz, 2H), 7.03-6.92 (m, 3H), 6.88-6.81 (m, 2H), 6.77 (d, J=2.4 Hz, 1H), 6.61 (d. J=8.3 Hz, 2H), 5.23-5.15 (m, 1H), 5.10-5.03 (m, 4H), 4.05 (d, J=3.0 Hz, 1H)

Step 3: To a solution of 7-(benzyloxy)-4-(4-bromophenyl)-3-(3-(trifluoromethoxy)phenyl) isochromane (210 mg, 1 eq., 378 μmol) and 4-(dimethoxymethyl)piperidine (90.3 mg, 1.5 eq., 567 μmol) in 1,4-Dioxane (8 mL) was added Cs₂CO₃ (370 mg, 3 eq., 1.13 mmol) and (2-Dicyclohexylphosphino-2′,6′-diisopropoxy-1,1′-biphenyl) [2-(2′-amino-1,1′-biphenyl)]palladium (II) methanesulfonate (31.6 mg, 0.1 eq., 37.8 μmol). The mixture was stirred under N₂ at 90° C. for 12 h. The reaction mixture was filtered and concentrated to yield a residue. The residue was purified by flash silica gel chromatography (from PE/EtOAc=1/0 to 20/1, TLC: PE/EtOAc=10/1, R_{f}=0.72) to yield 1-(4-(7-(benzyloxy)-3-(3-(trifluoromethoxy)phenyl)isochroman-4-yl)phenyl)-4-(dimethoxymethyl)piperidine (30 mg, 46 μmol, 12%, 97% Purity) was obtained as a white solid. LC-MS (ESI⁺) m/z: 634.2 (M+H)⁺.

¹H NMR (400 MHZ, CDCl₃) δ=7.47-7.39 (m, 4H), 7.37-7.32 (m, 1H), 7.23-7.16 (m, 1H), 7.03-6.94 (m, 3H), 6.83 (td, J=2.8, 5.4 Hz, 2H), 6.76 (d, J=2.4 Hz, 1H), 6.59 (br s, 4H), 5.20-5.14 (m, 1H), 5.09-5.00 (m, 4H), 4.06 (br d, J=7.0 Hz, 1H), 4.00 (br s, 1H), 3.55 (br d, J=12.2 Hz, 2H), 3.36 (s, 6H), 2.54 (br s, 2H), 1.92-1.65 (m, 3H), 1.39 (br d, J=10.6 Hz, 2H)

Step 4: To a solution of 1-(4-(7-(benzyloxy)-3-(3-(trifluoromethoxy)phenyl)isochroman-4-yl)phenyl)-4-(dimethoxymethyl)piperidine (160 mg, 1 eq., 252 μmol) in EtOAc (5 mL) was added Pd/C (160 mg, 10% Wt, 0.595 eq., 150 μmol). The mixture was stirred under H₂ 15 psi at 25° C. for 3 b. The reaction mixture was filtered and concentrated to yield a residue. The residue was separated by chiral SFC (column: DAICEL CHIRALPAK AD (250 mm*30 mm, 10 um); mobile phase: [TEA-i-PrOH]; B %: 40%-40%, min), followed by lyophilization to yield 7 (3R,4S)-4-(4-(4-(dimethoxymethyl)piperidin-1-yl)phenyl)-3-(3-(trifluoromethoxy)phenyl)isochroman-7-ol (50 mg, 92 μmol, 42%) SFC: Rt=1.136) and 7A (3S,4R)-4-(4-(4-(dimethoxymethyl)piperidin-1-yl)phenyl)-3-(3-(trifluoromethoxy)phenyl)isochroman-7-ol (50 mg, 92 μmol, 42%) SFC: Rt=2.085) as a white solid. LC-MS (ESI⁺) m/z: 544.1 (M+H)⁺.

¹H NMR of 7 (400 MHZ, CDCl₃) δ=7.23-7.16 (m, 1H), 6.97 (br d, J=7.6 Hz, 2H), 6.89-6.79 (m, 2H), 6.76-6.44 (m, 6H), 5.18-5.08 (m, 1H), 5.06-4.96 (m, 2H), 4.08 (br d, J=7.3 Hz, 1H), 3.98 (br s, 1H), 3.58-3.50 (m, 2H), 3.37 (s, 6H), 2.72-2.41 (m, 2H), 1.85-1.68 (m, 3H), 1.52-1.16 (m, 2H)

¹H NMR of 7A (400 MHz, CDCl₃) δ=7.22-7.17 (m, 1H), 6.98 (br d, J=7.6 Hz, 2H), 6.86 (br d, J=8.2 Hz, 1H), 6.81 (br s, 1H), 6.76-6.46 (m, 6H), 5.19-5.09 (m, 1H), 5.07-4.96 (m, 2H), 4.08 (br d, J=6.8 Hz, 1H), 3.98 (br s, 1H), 3.54 (br t, J=10.4 Hz, 2H), 3.37 (s, 6H), 2.77-2.39 (m, 2H), 1.86-1.64 (m, 3H), 1.44 (br s, 2H)

Step 5: To a solution of 7 (3R,4S)-4-(4-(4-(dimethoxymethyl)piperidin-1-yl)phenyl)-3-(3-(trifluoromethoxy)phenyl)isochroman-7-ol (40 mg, 1 eq., 74 μmol) in DCM (2 mL) was added TFA (0.7 g, 0.5 mL, 9e+1 eq., 6 mmol). The mixture was stirred at 25° C. for 1 h. The reaction mixture was quenched by add to saturation solution of NaHCO₃ (20 mL), extracted with EtOAc (10 mL×3). The combined organic layers were washed with brine (10 mL), dried over Na₂SO₄, filtered and concentrated under reduced pressure to yield 8 1-(4-((3R,4S)-7-hydroxy-3-(3-(trifluoromethoxy)phenyl)isochroman-4-yl)phenyl)piperidine-4-carbaldehyde (40 mg, 72 μmol, 98%, 90% Purity) as a yellow solid which was used in the next step without further purification. LC-MS (ESI⁺) m/z: 498.1 (M+H)⁺.

Step 6: To a solution of 7A (3S,4R)-4-(4-(4-(dimethoxymethyl)piperidin-1-yl)phenyl)-3-(3-(trifluoromethoxy)phenyl)isochroman-7-ol (40 mg, 1 eq., 74 μmol) in DCM (2 mL) was added TFA (0.7 g, 0.5 mL, 9e+1 eq., 6 mmol). The mixture was stirred at 25° C. for 1 h. The reaction mixture was quenched by add to saturation solution of NaHCO₃ (20 mL), extracted with EtOAc (10 mL×3). The combined organic layers were washed with brine (10 mL), dried over Na₂SO₄, filtered and concentrated under reduced pressure to yield 8A 1-(4-((3S,4R)-7-hydroxy-3-(3-(trifluoromethoxy)phenyl)isochroman-4-yl)phenyl)piperidine-4-carbaldehyde (40 mg, 72 μmol, 98%, 90% Purity) as a yellow solid which was used in the next step without further purification. LC-MS (ESI⁺) m/z: 498.1 (M+H)⁺.

Step 7: To a solution of 8, 1-(4-((3R,4S)-7-hydroxy-3-(3-(trifluoromethoxy)phenyl)isochroman-4-yl)phenyl)piperidine-4-carbaldehyde (40 mg, 1 eq., 80 μmol) and (S)-3-(1-oxo-5-(piperazin-1-yl)isoindolin-2-yl)piperidine-2,6-dione (53 mg, 2 eq., 0.16 mmol) in DCM (2 mL) and MeOH (2 mL), after stirred 30 min was added Sodium triacetoxyborohydride (51 mg, 36 μL, 3 eq., 0.24 mmol). The mixture was stirred at 25° C. for 12 h. The reaction mixture was filtered and concentrated to yield a residue. The residue was purified by preparative HPLC (column: Boston Green ODS 150*30 mm*5 um; mobile phase: [water (FA)-CAN]; B %: 18%-38%, 12 min), followed by lyophilization to yield 1-361 (S)-3-(5-(4-((1-(4-((3R,4S)-7-hydroxy-3-(3-(trifluoromethoxy)phenyl)isochroman-4-yl)phenyl)piperidin-4-yl)methyl)piperazin-1-yl)-1-oxoisoindolin-2-yl)piperidine-2,6-dione (22.9 mg, 28.3 μmol, 35%, 100% Purity) as a white solid. LC-MS (ESI⁺) m/z: 810.3 (M+H)⁺.

LCMS: calc. for C₄₅H₄₆F₃N₅O₆: 809.34, found: [M+H]⁺810.3.

HPLC: 100% purity at 220 nm.

¹H NMR (400 MHZ, DMSO-d₆) δ=10.95 (s, 1H), 9.31 (s, 1H), 8.13 (s, 1H), 7.51 (d, J=8.6 Hz, 1H), 7.33-7.28 (m, 1H), 7.19 (d, J=7.9 Hz, 1H), 7.10-7.02 (m, 3H), 6.88 (s, 1H), 6.78 (d, J=8.1 Hz, 1H), 6.60-6.52 (m, 6H), 5.15-4.99 (m, 3H), 4.92 (br d, J=15.3 Hz, 1H), 4.38-4.28 (m, 1H), 4.24-4.16 (m, 1H), 4.05 (d, J=2.9 Hz, 1H), 3.49 (br d, J=10.3 Hz, 2H), 3.31-3.26 (m, 8H), 2.96-2.83 (m, 1H), 2.60 (br s, 2H), 2.43-2.32 (m, 2H), 2.18 (br s, 2H), 2.00-1.93 (m, 1H), 1.73 (br d, J=12.3 Hz, 2H), 1.63 (br s, 1H), 1.19-1.08 (m, 2H)

Step 8: To a solution of 8A, 1-(4-((3S,4R)-7-hydroxy-3-(3-(trifluoromethoxy)phenyl)isochroman-4-yl)phenyl)piperidine-4-carbaldehyde (40 mg, 1 eq., 80 μmol) and (S)-3-(1-oxo-5-(piperazin-1-yl)isoindolin-2-yl)piperidine-2,6-dione (40 mg, 1.5 eq., 0.12 mmol) in DCM (2 mL) and MeOH (2 mL), after stirred 30 min was added Sodium triacetoxyborohydride (51 mg, 36 μL, 3 eq., 0.24 mmol). The mixture was stirred at 25° C. for 25 h. The reaction mixture was filtered and concentrated to yield a residue. The residue was purified by preparative HPLC (column: Boston Green ODS 150*30 mm*5 um; mobile phase: [water (FA)-CAN]; B %: 18%-38%, 12 min), followed by lyophilization to yield 1-362 (S)-3-(5-(4-((1-(4-((3S,4R)-7-hydroxy-3-(3-(trifluoromethoxy)phenyl)isochroman-4-yl)phenyl)piperidin-4-yl)methyl)piperazin-1-yl)-1-oxoisoindolin-2-yl)piperidine-2,6-dione (22.1 mg, 27.3 μmol, 34%, 100% Purity) as a white solid. LC-MS (ESI⁺) m/z: 810.3 (M+H)⁺.

LCMS: calc. for C₄₅H₄₆F₃N₅O₆: 809.34, found: [M+H]⁺810.3.

HPLC: 100% purity at 220 nm.

¹H NMR (400 MHz, DMSO-d₆) δ=10.94 (s, 1H), 9.31 (s, 1H), 8.14 (s, 1H), 7.51 (d, J=8.5 Hz, 1H), 7.33-7.28 (m, 1H), 7.19 (d, J=7.9 Hz, 1H), 7.11-7.02 (m, 3H), 6.88 (s, 1H), 6.78 (d, J=8.1 Hz, 1H), 6.60-6.53 (m, 6H), 5.12-5.01 (m, 3H), 4.92 (br d, J=15.3 Hz, 1H), 4.37-4.28 (m, 1H), 4.24-4.15 (m, 1H), 4.05 (d, J=2.7 Hz, 1H), 3.49 (br d, J=11.2 Hz, 2H), 3.31-3.27 (m, 8H), 2.95-2.84 (m, 1H), 2.58 (br d, J=18.6 Hz, 2H), 2.43-2.30 (m, 2H), 2.18 (br d, J=6.2 Hz, 2H), 2.01-1.92 (m, 1H), 1.73 (br d, J=12.5 Hz, 2H), 1.63 (br s, 1H), 1.13 (br d, J=11.2 Hz, 2H).

### Example 120. Preparation of (I-253) (S)-3-(5-(4-((1-(4-((1R,3S,4R)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione

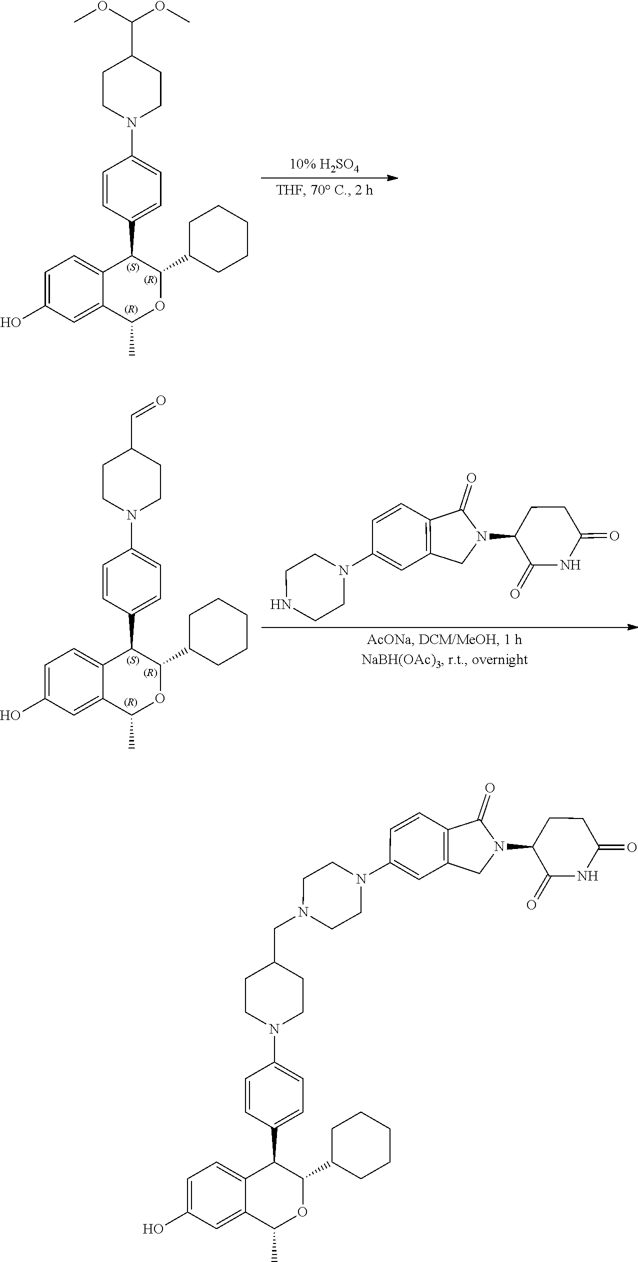

Step 1: A solution of 1-(4-((1R,3S,4R)-3-cyclohexyl-7-hydroxy-1-methylisochroman-4-yl)phenyl)piperidine-4-carbaldehyde (8 mg, 0.02 mmol, 82% purity) in 10% H₂SO₄ (1 mL) and THF (1 mL) was stirred at 70° C. for 2 hour. The reaction was adjust to pH=7 with saturated aqueous NaHCO₃, then 50 mL water was added, and the mixture was extracted with dichloromethane (100 mL*2). The combined organic layers were washed with brine (10 mL), dried over anhydrous Na₂SO₄, filtered and concentrated to dryness in vacuo to give a yellow oil. The yellow oil was concentrated in vacuo to give 1-(4-((1R, 3S, 4R)-3-cyclohexyl-7-hydroxy-1-methylisochroman-4-yl)phenyl)piperidine-4-carbaldehyde (8 mg, 0.02 mmol, 82% Purity) as a yellow solid.

Step 2: To a solution of 1-(4-((1R,3S,4R)-3-cyclohexyl-7-hydroxy-1-methylisochroman-4-yl)phenyl)piperidine-4-carbaldehyde (8 mg, 82% purity, 1 Eq, 0.02 mmol), (S)-3-(1-oxo-5-(piperazin-1-yl)isoindolin-2-yl)piperidine-2,6-dione (6 mg, 1.1 Eq, 0.02 mmol, HCl salt) in DCM (1 mL), MeOH (1 mL) was added sodium acetate (4 mg, 3 Eq, 0.05 mmol) was stirred at 25° C. for 60 mins, Then sodium triacetoxyhydroborate (6 mg, 2 Eq, 0.03 mmol) and acetic acid (3 mg, 3 μL, 3 Eq, 0.05 mmol) was added. The mixture was stirred at 25° C. for 15 hour. The reaction mixture was concentrated under reduced pressure to remove solvent. The residue was purified by prep-HPLC (column: Welch Xtimate C18 150*30 mm*5 um phase: [water (FA)-ACN]; B %: 15%-55%,9 min to give (S)-3-(5-(4-((1-(4-((1R,3S,4R)-3-cyclohexyl-7-hydroxy-1-methylisochroman-4-yl)phenyl)piperidin-4-yl)methyl)piperazin-1-yl)-1-oxoisoindolin-2-yl)piperidine-2,6-dione (2 mg, 3 μmol, 20% yield, 96.6% purity) as a white solid.

LCMS: calc. for C₄₅H₅₅N₅O₅: 745.42, found: [M+H]⁺746.5.

HPLC: 96.6% purity at 220 nm.

¹H NMR (400 MHZ, DMSO-d₆) δ 8.42-8.46 (m, 0.218H) 7.67 (d, J=8.34 Hz, 1H) 7.09-7.16 (m, 2H) 6.94-7.03 (m, 4H) 6.58 (d, J=1.79 Hz, 1H) 6.44-6.52 (m, 1H) 6.44-6.52 (m, 1H) 5.13 (dd, J=13.35, 5.25 Hz, 1H) 4.82-4.91 (m, 2H) 4.35-4.50 (m, 2H) 4.00 (d, J=10.25 Hz, 1H) 3.63-3.74 (m, 2H) 3.50-3.55 (m, 1H) 3.40-3.48 (m, 4H) 2.87-2.97 (m, 1H) 2.64-2.84 (m, 7H) 2.39-2.55 (m, 3H) 2.11-2.22 (m, 1H) 1.92-2.00 (m, 2H) 1.62-1.91 (m, 5H) 1.52 (d, J=6.44 Hz, 4H) 1.33-1.46 (m, 3H) 1.27 (br t, J=10.79 Hz, 2H) 1.11-1.16 (m, 2H).

The absolute stereochemistry was arbitrarily assigned.

### Example 121. Preparation of (I-365) (S)-3-(5-(4-((1-(4-((3S,4R)-3-(2,3-dihydro-1H-inden-5-yl)-7-hydroxyisochroman-4-yl)phenyl)piperidin-4-yl)methyl)piperazin-1-yl)-1-oxoisoindolin-2-yl)piperidine-2,6-dione and (I-366) (S)-3-(5-(4-((1-(4-((3R,4S)-3-(2,3-dihydro-1H-inden-5-yl)-7-hydroxyisochroman-4-yl)phenyl)piperidin-4-yl)methyl)piperazin-1-yl)-1-oxoisoindolin-2-yl)piperidine-2,6-dione

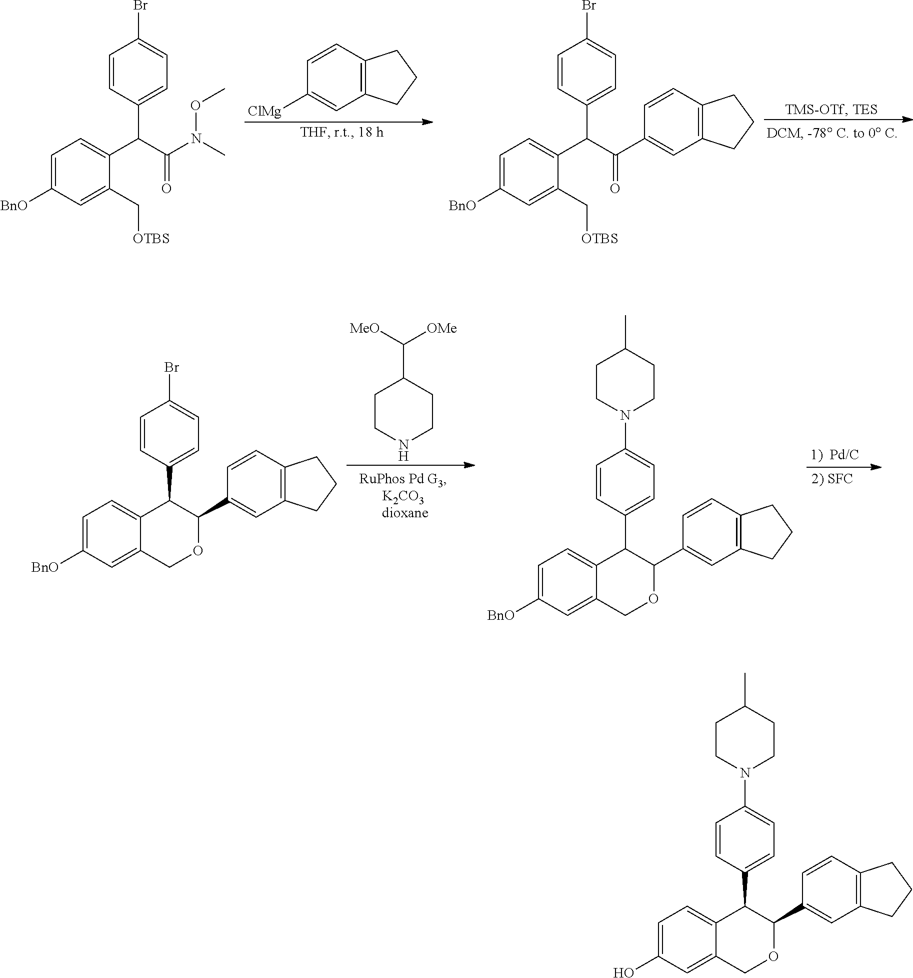

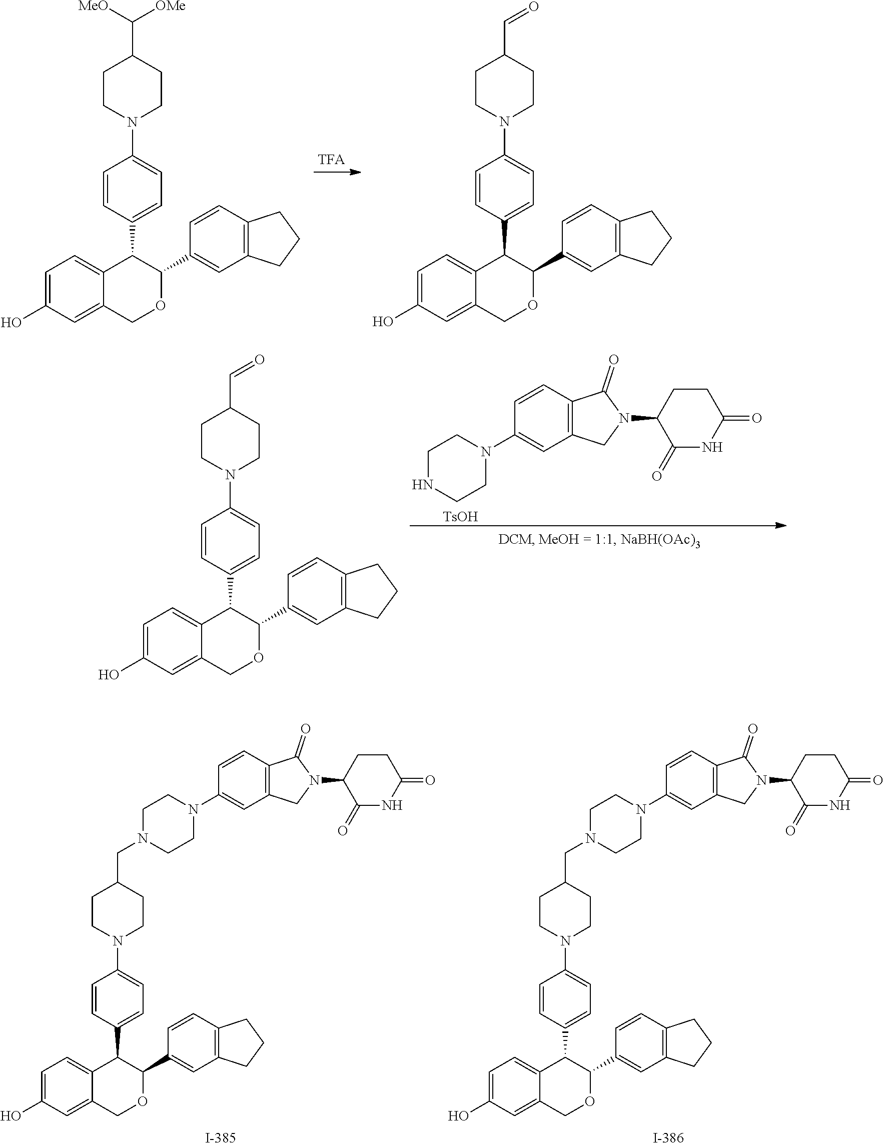

Step 1: To a solution of 5-iodoindane (860.81 mg, 3.42 mmol, 2 eq.) in THF (10 mL) was added chloro(isopropyl) magnesium (2 M, 1.88 mL, 2.2 eq.) at 0° C., after stirred 1 h was added 2-[4-benzyloxy-2-[tert-butyl(dimethyl)silyl]oxymethyl]phenyl]-2-(4-bromophenyl)-N-methoxy-N-methyl-acetamide (1 g, 1.71 mmol, 1 eq.) in THF (10 mL). The mixture was stirred at 25° C. for 12 h. The reaction mixture was quenched by addition of water (30 mL), extracted with EtOAc (30 mL×3). The combined organic layers were washed with brine (30 mL.), dried over Na₂SO₄, filtered and concentrated under reduced pressure to give a residue. The residue was purified by flash silica gel chromatography (from PE/EtOAc=1/0 to 10/1, TLC: PE/EtOAc=5/1, R_{f}=0.79) to yield 2-[4-benzyloxy-2-[tert-butyl(dimethyl)silyl]oxymethyl]phenyl]-2-(4-bromophenyl)-1-indan-5-yl-ethanone (750 mg, 1.06 mmol, 62.18% yield, 91% purity) as colourless oil. LC-MS (ESI⁺) m/z: 642.1 (M+H)⁺.

¹H NMR (400 MHZ, CDCl₃) δ=7.87-7.79 (m, 2H), 7.45-7.30 (m, 7H), 7.20 (d, J=7.9 Hz, 1H), 7.05-6.98 (m, 4H), 6.81 (dd, J=2.8, 8.5 Hz, 1H), 6.31 (s, 1H), 5.03 (s, 2H), 4.70 (d, J=12.5 Hz, 1H), 4.49 (d, J=12.5 Hz, 1H), 2.94-2.88 (m, 4H), 2.13-2.06 (m, 2H), 0.93 (s, 9H), 0.10-0.04 (m, 6H)

Step 2: To a solution of 2-[4-benzyloxy-2-[tert-butyl(dimethyl)silyl]oxymethyl]phenyl]-2-(4-bromophenyl)-1-indan-5-yl-ethanone (750 mg, 1.06 mmol, 1 eq.) and triethylsilane (185.51 mg, 1.60 mmol, 254.82 μL, 1.5 eq.) in DCM (10 mL) was added trimethylsilyl trifluoromethanesulfonate (354.58 mg, 1.60 mmol, 288.28 μL, 1.5 eq.) at −78° C. The mixture was stirred at −78° C. for 30 min. The reaction mixture was quenched by addition to saturation solution of NaHCO₃ (50 mL), extracted with DCM (30 mL×3). The combined organic layers were washed with brine (20 mL), dried over Na₂SO₄, filtered and concentrated under reduced pressure to give a residue. The residue was purified by flash silica gel chromatography (from PE/EtOAc=1/0 to 20/1, TLC: PE/EtOAc=10/1, R_{f}=0.70) to afford 7-benzyloxy-4-(4-bromophenyl)-3-indan-5-yl-isochromane (450 mg, 853.46 μmol, 80.24% yield, 97% purity) as a yellow solid. LC-MS (ESI⁺) m/z: 511.1 (M+H)⁺.

¹H NMR (400 MHz, CDCl₃) δ=7.47-7.33 (m, 5H), 7.16 (d, J=8.5 Hz, 2H), 7.02 (d, J=7.7 Hz, 1H), 6.94 (d, J=8.5 Hz, 1H), 6.85-6.80 (m, 1H), 6.79-6.71 (m, 3H), 6.65 (d, J=8.5 Hz, 2H), 5.20-5.13 (m, 1H), 5.09-5.01 (m, 4H), 4.03 (d, J==2.9 Hz, 1H), 2.85-2.68 (m, 4H), 2.01 (quin, J=7.4 Hz, 2H)

Step 3: To a solution of 7-(benzyloxy)-4-(4-bromophenyl)-3-(2,3-dihydro-1H-inden-5-yl)isochromane (510 mg, 1 eq., 997 μmol) and 4-(dimethoxymethyl)piperidine (238 mg, 1.5 eq., 1.50 mmol) in 1,4-Dioxane (10 mL) was added Cs₂CO₃ (812 mg, 2.5 eq., 2.49 mmol) and (2-Dicyclohexylphosphino-2′,6′-diisopropoxy-1,1′-biphenyl) [2-(2′-amino-1,l′-biphenyl)]palladium (II) methanesulfonate (83.4 mg, 0.1 eq., 99.7 μmol). The mixture was stirred under N₂ at 90° C. for 12 h. The reaction mixture was quenched by addition of water (50 mL), filtered and extracted with EtOAc (30 mL×3). The combined organic layers were washed with brine (10 mL), dried over Na₂SO₄, filtered, and concentrated under reduced pressure to give a residue. The residue was purified by flash silica gel chromatography (from PE/EtOAc=1/0 to 5/1, TLC: PE/EtOAc=3/1, R_{f}=0.49) to afford 1-(4-(7-(benzyloxy)-3-(2,3-dihydro-1H-inden-5-yl)isochroman-4-yl)phenyl)-4-(dimethoxymethyl)piperidine (450 mg, 0.75 mmol, 75%, 98% Purity) as a yellow solid. LC-MS (ESI⁺) m/z: 590.0 (M+H)⁺.

¹H NMR (400 MHZ, CDCl₃) δ=7.47-7.32 (m, 4H), 7.37-7.31 (m, 1H), 6.99 (t, J=7.6 Hz, 2H), 6.82 (dd, J=2.6, 8.4 Hz, 1H), 6.79-6.59 (m, 7H), 5.19-5.13 (m, 1H), 5.10-4.97 (m, 4H), 4.07 (br d, J=7.1 Hz, 1H), 3.99 (br s, 1H), 3.60-3.53 (m, 1H), 3.57 (br d, J=11.9 Hz, 1H), 3.36 (s, 6H), 2.90-2.65 (m, 4H), 2.55 (br s, 2H), 2.00 (quin, J=7.1 Hz, 2H), 1.81 (br d, J=10.8 Hz, 2H), 1.70 (br s, 1H), 1.43 (br s, 2H)

Step 4: To a solution of 1-(4-(7-(benzyloxy)-3-(2,3-dihydro-1H-inden-5-yl)isochroman-4-yl)phenyl)-4-(dimethoxymethyl)piperidine (200 mg, 1 eq., 339 μmol) in EtOAc (5 mL) was added Pd/C (200 mg, 10% Wt, 0.554 eq., 188 μmol). The mixture was stirred under H₂ (15 psi) at 25° C. for 3 h. The reaction mixture was filtered and concentrated to yield a residue. The residue was separated by chiral SFC (column: DAICEL CHIRALPAK AD (250 mm*30 mm, 10 um); mobile phase: [CO₂-i-PrOH]; B %: 40%-40%, min), followed by lyophilization to yield (3S,4R)-3-(2,3-dihydro-1H-inden-5-yl)-4-(4-(4-(dimethoxymethyl)piperidin-1-yl)phenyl)isochroman-7-ol (70 mg, 0.14 mmol, SFC: Rt=1.870) as a white solid and (3R,4S)-3-(2,3-dihydro-1H-inden-5-yl)-4-(4-(4-(dimethoxymethyl)piperidin-1-yl)phenyl)isochroman-7-ol (70 mg, 0.14 mmol, SFC: Rt=3.617) as a white solid. LC-MS (ESI⁺) m/z: 500.1 (M+H)⁺.

¹H NMR of 7 (500 MHz, CDCl₃) δ=7.00 (d, J=7.6 Hz, 1H), 6.89 (d, J=8.2 Hz, 1H), 6.77-6.71 (m, 2H), 6.69-6.53 (m, 6H), 5.12 (br d, J=15.4 Hz, 2H), 5.04-4.96 (m, 2H), 4.13-4.05 (m, 1H), 4.00-3.94 (m, 1H), 3.60-3.53 (m, 2H), 3.37 (s, 6H), 2.82 (t, J=7.3 Hz, 2H), 2.80-2.74 (m, 1H), 2.73-2.65 (m, 1H), 2.63-2.44 (m, 2H), 1.99 (spt, J=6.9 Hz, 2H), 1.81 (br d, J=9.6 Hz, 2H), 1.71 (br s, 1H), 1.44 (br s, 2H)

¹H NMR of 7A (500 MHz, CDCl₃) δ=7.00 (d, J=7.6 Hz, 1H), 6.89 (d, J=8.2 Hz, 1H), 6.77-6.71 (m, 2H), 6.70-6.52 (m, 6H), 5.12 (br d, J=15.4 Hz, 2H), 5.03-4.96 (m, 2H), 4.10-4.04 (m, 1H), 4.00-3.93 (m, 1H), 3.57 (br dd, J=3.5, 8.7 Hz, 2H), 3.37 (s, 6H), 2.82 (t, J=7.3 Hz, 2H), 2.79-2.74 (m, 1H), 2.72-2.65 (m, 1H), 2.62-2.45 (m, 2H), 2.04-1.96 (m, 2H), 1.82 (br d. J=10.2 Hz, 2H), 1.71 (br d, J=3.7 Hz, 1H), 1.52-1.32 (m, 2H)

Step 5: To a solution of (3S,4R)-3-(2,3-dihydro-1H-inden-5-yl)-4-(4-(4-(dimethoxymethyl) piperidin-1-yl)phenyl)isochroman-7-ol (40 mg, 1 eq., 80 μmol) in DCM (2 mL) was added TFA (0.7 g, 0.5 mL, 8e+1 eq., 6 mmol). The mixture was stirred at 25° C. for 1 h. The reaction mixture was quenched by add to saturation solution of NaHCO₃ (20 mL), extracted with EtOAc (10 mL×3). The combined organic layers were washed with brine (10 mL), dried over Na₂SO₄, filtered and concentrated under reduced pressure to yield 1-(4-((3S,4R)-3-(2,3-dihydro-1H-inden-5-yl)-7-hydroxyisochroman-4-yl)phenyl)piperidine-4-carbaldehyde (40 mg, 79 μmol, 99%, 90% Purity) as a yellow solid which was used in the next step without further purification. LC-MS (ESI⁺) m/z: 454.1 (M+H)⁺.

Step 6: To a solution of (3R,4S)-3-(2,3-dihydro-1H-inden-5-yl)-4-(4-(4-(dimethoxymethyl) piperidin-1-yl)phenyl)isochroman-7-ol (40 mg, 1 eq., 80 μmol) in DCM (2 mL) was added TFA (0.7 g, 0.5 mL, 8e+1 eq., 6 mmol). The mixture was stirred at 25° C. for 1 h. The reaction mixture was quenched by add to saturation solution of NaHCO₃ (20 mL), extracted with EtOAc (10 mL×3). The combined organic layers were washed with brine (10 mL), dried over Na₂SO₄, filtered and concentrated under reduced pressure to yield 1-(4-((3R,4S)-3-(2,3-dihydro-1H-inden-5-yl)-7-hydroxyisochroman-4-yl)phenyl)piperidine-4-carbaldehyde (40 mg, 79 μmol, 99%, 90% Purity) as a yellow solid which was used in the next step without further purification. LC-MS (ESI⁺) m/z: 454.1 (M+H)⁺.

Step 7: To a solution of 1-(4-((3S,4R)-3-(2,3-dihydro-1H-inden-5-yl)-7-hydroxyisochroman-4-yl)phenyl)piperidine-4-carbaldehyde (40 mg, 1 eq., 88 μmol) and (S)-3-(1-oxo-5-(piperazin-1-yl)isoindolin-2-yl)piperidine-2,6-dione (58 mg, 2 eq., 0.18 mmol) in DCM (2 mL) and MeOH (2 mL), after stirred 30 min was added Sodium triacetoxyborohydride (56 mg, 39 μL, 3 eq., 0.26 mmol). The mixture was stirred at 25° C. for 12 h. The reaction mixture was filtered and concentrated to yield a residue. The residue was purified by preparative HPLC (column: Boston Green ODS 150*30 mm*5 um; mobile phase: [water (FA)-CAN]; B %: 18%-38%, 12 min), followed by lyophilization to yield (S)-3-(5-(4-((1-(4-((3S,4R)-3-(2,3-dihydro-1H-inden-5-yl)-7-hydroxyisochroman-4-yl)phenyl)piperidin-4-yl)methyl)piperazin-1-yl)-1-oxoisoindolin-2-yl)piperidine-2,6-dione (16.3 mg, 21.3 μmol, 24%, 100% Purity) as a white solid. LC-MS (ESI⁺) m/z: 766.3 (M+H)⁺.

LCMS: calc. for C₄₇H₅₁N₅O₅: 765.39, found: [M+H]⁺766.3.

HPLC: 100% purity at 220 nm.

¹H NMR (400 MHz, DMSO-d₆) δ=10.94 (s, 1H), 9.27 (s, 1H), 8.16 (s, 1H), 7.51 (d, J=8.6 Hz, 1H), 7.08-7.01 (m, 2H), 6.97 (d, J=7.7 Hz, 1H), 6.88 (s, 1H), 6.84-6.73 (m, 2H), 6.69-6.62 (m, 2H), 6.59-6.51 (m, 4H), 5.08-5.00 (m, 2H), 4.95-4.85 (m, 2H), 4.36-4.29 (m, 1H), 4.23-4.16 (m, 1H), 3.99 (br s, 1H), 3.51 (br d, J=12.4 Hz, 2H), 3.27 (br s, 6H), 2.96-2.84 (m, 1H), 2.81-2.54 (m, 8H), 2.40-2.29 (m, 2H), 2.18 (br d, J=7.0 Hz, 2H), 2.00-1.88 (m, 3H), 1.74 (br d, J=11.6 Hz, 2H), 1.63 (br s, 1H), 1.14 (br d, J=10.8 Hz, 2H)

Step 8: To a solution of 1-(4-((3R,4S)-3-(2,3-dihydro-1H-inden-5-yl)-7-hydroxyisochroman-4-yl)phenyl)piperidine-4-carbaldehyde (40 mg, 1 eq., 88 μmol) and (S)-3-(1-oxo-5-(piperazin-1-yl)isoindolin-2-yl)piperidine-2,6-dione (58 mg, 2 eq., 0.18 mmol) in DCM (2 mL) and MeOH (2 mL), after stirred 30 min was added Sodium triacetoxyborohydride (56 mg, 39 μL, 3 eq., 0.26 mmol). The mixture was stirred at 25° C. for 25 h. The reaction mixture was filtered and concentrated to yield a residue. The residue was purified by preparative HPLC (column: Boston Green ODS 150*30 mm*5 um; mobile phase: [water (FA)-CAN]; B %: 18%-48%, 12 min), followed by lyophilization to yield (S)-3-(5-(4-((1-(4-((3R,4S)-3-(2,3-dihydro-1H-inden-5-yl)-7-hydroxyisochroman-4-yl)phenyl)piperidin-4-yl)methyl)piperazin-1-yl)-1-oxoisoindolin-2-yl)piperidine-2,6-dione (13.1 mg, 17.1 μmol, 19%, 100% Purity) as a white solid. LC-MS (ESI⁺) m/z: 766.3 (M+H)⁺.

LCMS: calc. for C₄₇H₅₁N₅O₅:765.39, found: [M+H]⁺766.3.

HPLC: 100% purity at 220 nm.

¹H NMR (400 MHZ, DMSO-d₆) δ=10.94 (s, 1H), 9.27 (br s, 1H), 8.18 (s, 1H), 7.51 (d, J=8.3 Hz, 1H), 7.08-7.02 (m, 2H), 6.97 (d, J=7.9 Hz, 1H), 6.88 (s, 1H), 6.84-6.76 (m, 2H), 6.68-6.63 (m, 2H), 6.60-6.52 (m, 4H), 5.09-5.01 (m, 2H), 4.95-4.85 (m, 2H), 4.36-4.28 (m, 1H), 4.23-4.16 (m, 1H), 3.99 (d, J=2.9 Hz, 1H), 3.51 (br d, J=11.8 Hz, 2H), 3.27 (br s, 6H), 2.96-2.84 (m, 1H), 2.80-2.53 (m, 8H), 2.44-2.28 (m, 2H), 2.18 (br d, J=7.0 Hz, 2H), 1.99-1.89 (m, 3H), 1.74 (br d, J=12.2 Hz, 2H), 1.63 (br s, 1H), 1.14 (br d, J=11.0 Hz, 2H).

### Example 122. Preparation of (I-225) (S)-3-(5-(4-((1-(4-((3R,4R)-7-hydroxy-3-isopropylisochroman-4-yl)phenyl)piperidin-4-yl)methyl)piperazin-1-yl)-1-oxoisoindolin-2-yl)piperidine-2,6-dione

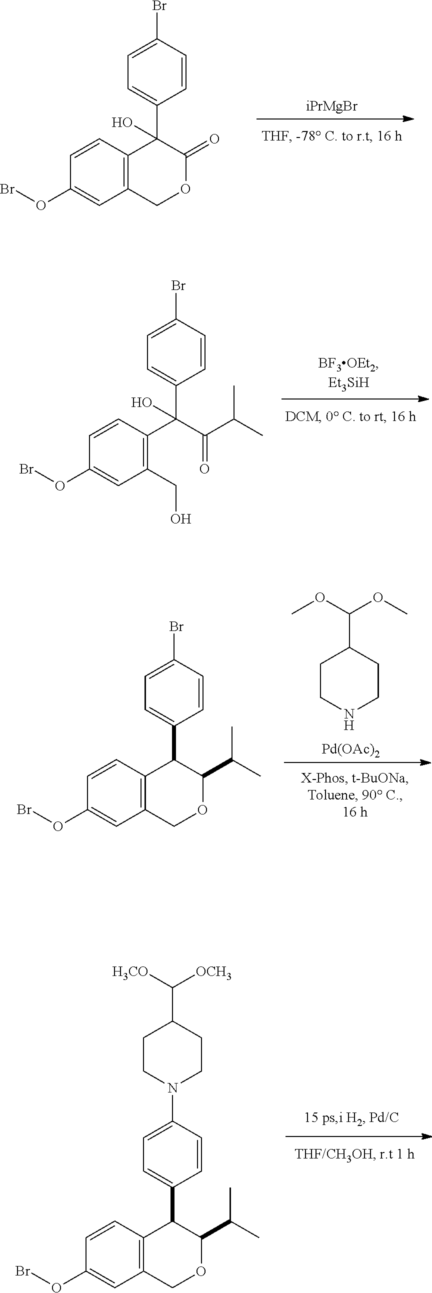

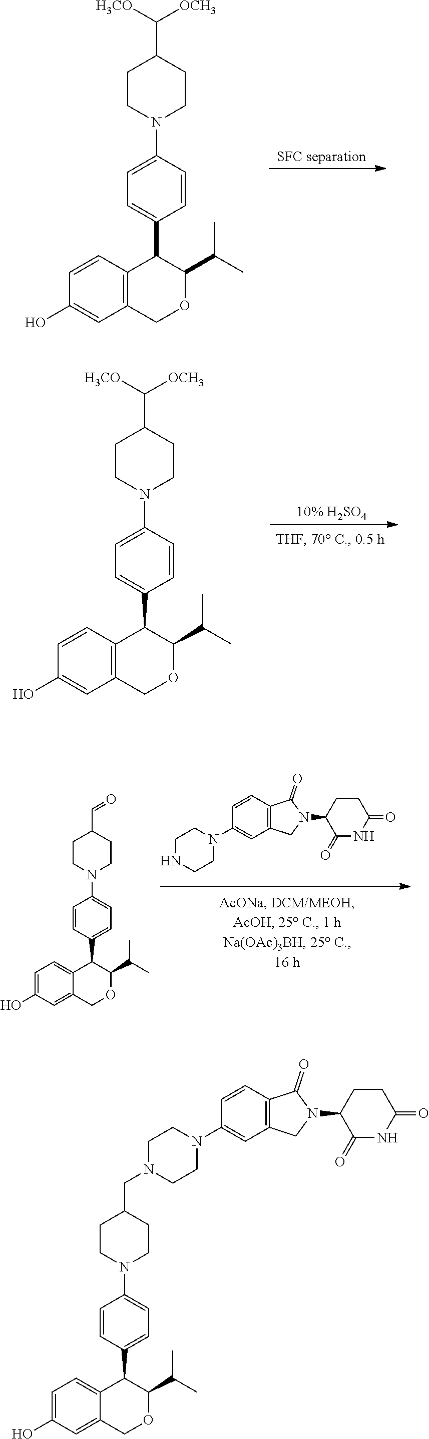

Step 1: A mixture of 7-(benzyloxy)-4-(4-bromophenyl)-4-hydroxyisochroman-3-one (5 g, 1 Eq, 12 mmol) in THF (50 mL) was stirred at −78° C. under N₂, then isopropyl magnesium bromide solution (7.6 g, 52 mL, 1 M, 4.4 Eq, 52 mmol) was added to the mixture and stirred at −78° C. for 3 h. Then the reaction was warmed to r.t. and stirred for 13 h. The reaction was quenched with saturated aqueous NH₄Cl (50 mL) at ice bath, then the mixture was added H₂O (100 mL) and extracted with ethyl acetate (200 mL*2). The combined extracts were dried over anhydrous Na₂SO₄, filtered, and concentrated to dryness in vacuum to give a residue. The residue was subjected to column chromatography over silica gel (petroleum ether/ethyl acetate from 100/0 to 70/30). The pure fractions were collected and concentrated to dryness in vacuum to give 1-(4-(benzyloxy)-2-(hydroxymethyl)phenyl)-1-(4-bromophenyl)-1-hydroxy-3-methylbutan-2-one (1.8 g, 29% yield, 89.4% purity) as a yellow solid. LCMS (ESI⁺) m/z: 451.1 (M−18)⁺.

Step 2: A mixture of 1-(4-(benzyloxy)-2-(hydroxymethyl)phenyl)-1-(4-bromophenyl)-1-hydroxy-3-methylbutan-2-one (1.7 g, 1 Eq, 3.2 mmol) in DCM (30 mL) was stirred at 0° C. under N₂, then triethylsilane (1.9 g, 2.6 mL, 5 Eq, 16 mmol) and boron trifluoride etherate (920 mg, 0.82 mL, 2 Eq, 6.5 mmol) were added to the mixture at 0° C. Then the reaction was warmed to r.t. and stirred for 16 h. The reaction was quenched with saturated aqueous NaHCO₃ (30 mL) at ice bath, then the mixture was added H₂O (100 mL) and extracted with ethyl acetate (100 mL*2). The combined extracts were dried over anhydrous Na₂SO₄, filtered, and concentrated to dryness in vacuum to give a residue. The residue was subjected to column chromatography over silica gel (petroleum ether/ethyl acetate from 100/0 to 70/30). The pure fractions were collected and concentrated to dryness in vacuum to give 7-(benzyloxy)-4-(4-bromophenyl)-3-isopropylisochromane (1.4 g, 99% yield) as a white solid.

Step 3: A mixture of 7-(benzyloxy)-4-(4-bromophenyl)-3-isopropylisochromane (1.4 g, 1 Eq, 3.2 mmol), 2-(Dicyclohexylphosphanyl)-2′,4′,6′-tris(isopropyl) biphenyl (0.31 g, 0.2 Eq, 0.64 mmol), palladium diacetate (0.11 g, 0.15 Eq, 0.48 mmol), 4-(dimethoxymethyl)piperidine (0.76 g, 0.8 mL, 1.5 Eq, 4.8 mmol) and sodium 2-methylpropan-2-olate (0.46 g, 1.5 Eq, 4.8 mmol) in toluene (30 mL) was degassed and purged with N₂ for 3 times at r.t. Then the reaction was heated at 90° C. for 16 h. The mixture was added H₂O (100 mL) and extracted with ethyl acetate (100 mL*2). The combined extracts were dried over anhydrous Na₂SO₄, filtered and concentrated to dryness in vacuum to give a residue. The residue was subjected to column chromatography over silica gel (petroleum ether/ethyl acetate from 100/0 to 80/20). The pure fractions were collected and concentrated to dryness in vacuum to give 1-(4-(7-(benzyloxy)-3-isopropylisochroman-4-yl)phenyl)-4-(dimethoxymethyl)piperidine (720 mg, 43% yield, 98.3% purity) as a white solid. LC-MS (ESI⁺) m/z: 516.2 (M+H)⁺.

Step 4: A mixture of 1-(4-(7-(benzyloxy)-3-isopropylisochroman-4-yl)phenyl)-4-(dimethoxymethyl)piperidine (720 mg, 1 Eq, 1.37 mmol), Pd/C (720 mg, 10% wt, 0.493 Eq, 677 μmol) and H₂ (2.77 mg, 1 Eq, 1.37 mmol) in THF (5 mL) and MeOH (5 mL) was degassed and purged with H₂ for 3 times at r.t., then the mixture was stirred at 25° C. for 1 h with H₂ atmosphere (15 psi). The mixture was filtered and concentrated to dryness in vacuum to give a white solid. The white solid was purified by SFC (column: DAICEL CHIRALPAK AD (250 mm*30 mm, 10 um); mobile phase: [CO₂-i-PrOH (0.1% NH₃H₂O)]; B % 40%-40%, 45 min). The aqueous phase was collected to dryness to give (3R, 4R)-4-(4-(4-(dimethoxymethyl) piperidin-1-yl)phenyl)-3-isopropylisochroman-7-ol (224 mg, 37.4% yield, 97.5% purity) as a white solid. LC-MS (ESI⁺) m/z: 426.2 (M+H)⁺.

Step 5: A mixture of (3R,4R)-4-(4-(4-(dimethoxymethyl)piperidin-1-yl)phenyl)-3-isopropylisochroman-7-ol (224 mg, 1 Eq, 513 μmol) in THF (3 mL) and 10% H₂SO₄ (3 mL) was stirred at 70° C. for 0.5 h. The reaction was adjust to pH=8 with saturated aqueous NaHCO₃ (10 mL) at 0° C. Then the mixture was added H₂O (20 mL) and extracted with ethyl acetate (20 mL*2). The combined organic layers were dried over anhydrous Na₂SO₄, filtered and concentrated to dryness in vacuum to give 1-(4-((3R,4R)-7-hydroxy-3-isopropylisochroman-4-yl)phenyl)piperidine-4-carbaldehyde (195 mg, 98.8% yield, 98.7% purity) as a yellow solid LC-MS (ESI⁺) m/z: 380.2 (M+H)⁺.

Step 6: A mixture of 1-(4-((3R,4R)-7-hydroxy-3-isopropylisochroman-4-yl)phenyl)piperidine-4-carbaldehyde (195 mg, 1 Eq, 507 μmol), (S)-3-(1-oxo-5-(piperazin-1-yl)isoindolin-2-yl)piperidine-2,6-dione HCl (222 mg, 1.2 Eq, 609 μmol), sodium acetate (208 mg, 5 Eq, 2.54 mmol) and acetic acid (91 mg, 87 μL, 3 Eq, 1.52 mmol) in THE (3 mL) and MeOH (3 mL) was stirred at 25° C. for 1 h, then sodium triacetoxyborohydride (215 mg, 2 Eq, 1.01 mmol) was added to the mixture and stirred at 25° C. for 16 h. Then the mixture was added H₂O (50 mL) and extracted with ethyl acetate (50 mL*2). The combined extracts were dried over anhydrous Na₂SO₄, filtered and concentrated to dryness in vacuum to give a residue. The residue was subjected to column chromatography over silica gel (petroleum ether/ethyl acetate from 100/0 to 0/100 and DCM/MeOH from 100/0 to 90/10). The pure fractions were collected and concentrated to dryness in vacuum and lyophilized to dryness to give (S)-3-(5-(4-((1-(4-((3R,4R)-7-hydroxy-3-isopropylisochroman-4-yl)phenyl)piperidin-4-yl)methyl)piperazin-1-yl)-1-oxoisoindolin-2-yl)piperidine-2,6-dione (167.2 mg, 45.7% yield, 95.9% purity) as a white solid. LC-MS (ESI⁺) m/z: 692.4 (M+H)⁺.

LCMS: calc. for C₄₁H₄₉N₅O₅:691.37, found: [M+H]⁺692.4.

HPLC: 95.898% purity at 220 nm.

¹HNMR (400 MHz, DMSO-d₆) δ=10.96 (s, 1H), 9.21 (br s, 1H), 7.52 (d, J=8.6 Hz, 1H), 7.12-7.04 (m, 4H), 6.77 (dd, J=6.6, 8.4 Hz, 3H), 6.50 (dd, J=2.3, 8.2 Hz, 1H), 6.44 (d, J=2.3 Hz, 1H), 5.05 (dd, J=5.1, 13.3 Hz, 1H), 4.89 (d, J=15.3 Hz, 1H), 4.67 (d, J=15.1 Hz, 1H), 4.37-4.29 (m, 1H), 4.24-4.17 (m, 1H), 3.76 (br s, 1H), 3.59 (br d, J=9.2 Hz, 2H), 3.31-3.22 (m, 5H), 2.91 (br t, J=12.8 Hz, 1H), 2.59 (br d, J=13.9 Hz, 5H), 2.50-2.46 (m, 2H), 2.44-2.33 (m, 1H), 2.22 (br d, J=7.3 Hz, 2H), 2.01-1.92 (m, 1H), 1.80 (br d, J=12.8 Hz, 2H), 1.67 (br s, 1H), 1.28-1.14 (m, 3H), 0.90 (d, J=6.4 Hz, 3H), 0.84 (d, J=6.7 Hz, 3H).

### Example 123. Preparation of (I-369) (S)-3-(5-(4-((1-(4-((3S,4R)-3-(2,5-difluoro-4-methylphenyl)-7-hydroxyisochroman-4-yl)phenyl)piperidin-4-yl)methyl)piperazin-1-yl)-1-oxoisoindolin-2-yl)piperidine-2,6-dione and (I-370) (S)-3-(5-(4-((1-(4-((3R,4S)-3-(2,5-difluoro-4-methylphenyl)-7-hydroxyisochroman-4-yl)phenyl)piperidin-4-yl)methyl)piperazin-1-yl)-1-oxoisoindolin-2-yl)piperidine-2,6-dione

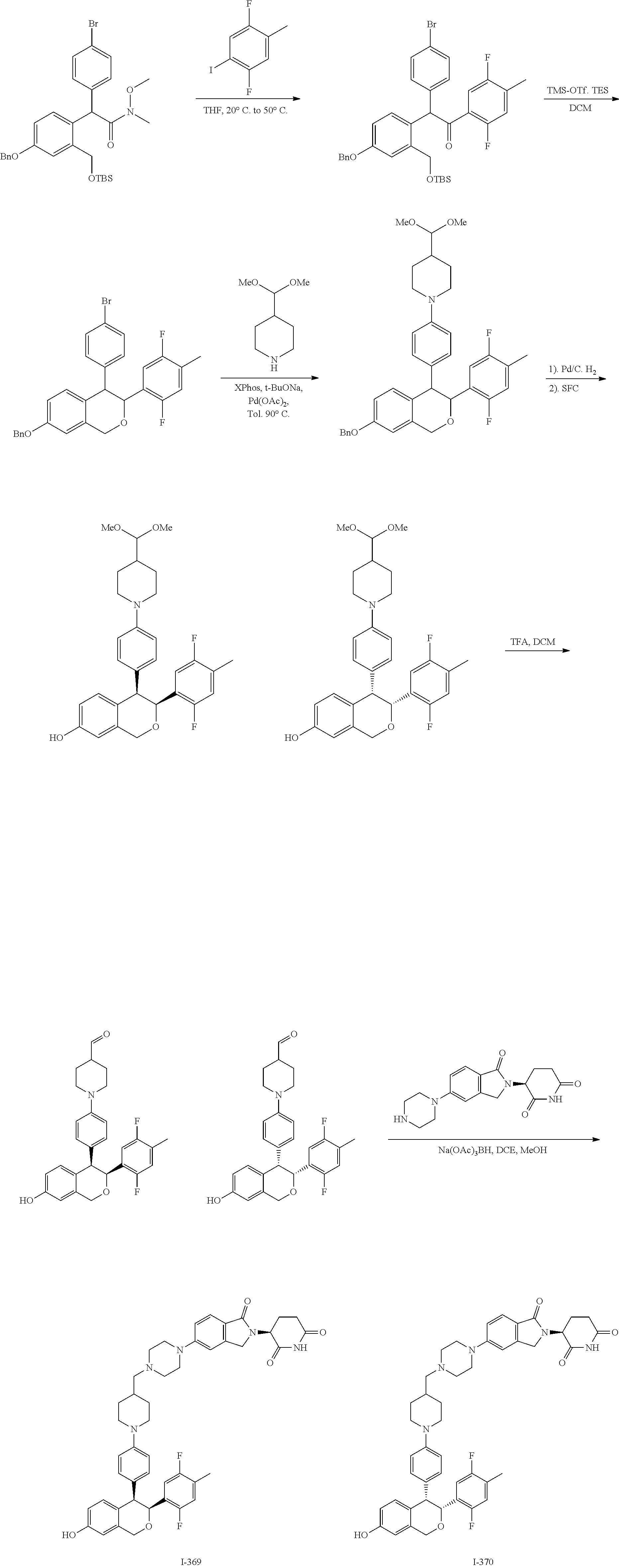

Step 1: To a solution of 1,4-difluoro-2-iodo-5-methylbenzene (521 mg, 4 eq, 2.05 mmol) in THF (3 mL) was added isopropylmagnesiumchloridelithiumchloridecomplex (374 μL, 1M, 5 eq, 2.57 mmol) dropwise at 20° C. and stirred at 20° C. for 0.5 h. Then a solvent of 2-(4-(benzyloxy)-2-(((tert-butyldimethylsilyl)oxy)methyl)phenyl)-2-(4-bromophenyl)-N-methoxy-N-methylacetamide (300 mg, 1 eq, 513 μmol) in THF (3 mL) was added dropwise, The mixture was stirred at 50° C. for 16 h. The reaction was quenched with sat.aq. NaHCO₃ (50 mL) and extracted with EtOAc (30 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 5%) to give a product of 2-(4-(benzyloxy)-2-(((tert-butyldimethylsilyl)oxy)methyl)phenyl)-2-(4-bromophenyl)-1-(2,5-difluoro-4-methylphenyl) ethan-1-one (270 mg, 73% yield) as a white solid

¹HNMR (400 MHz, CDCl₃): δ 7.54 (dd, J=5.8, 9.7 Hz, 1H), 7.44-7.34 (m, 7H), 7.12 (d, J=2.5 Hz, 1H), 7.03 (d, J=8.5 Hz, 2H), 6.93-6.85 (m, 2H), 6.80 (dd, J=2.6, 8.5 Hz, 1H), 6.03 (s, 1H), 5.04 (s, 2H), 4.76 (d, J=13.2 Hz, 1H), 4.56 (d, J=13.4 Hz, 1H), 2.28 (s, 3H), 0.89 (s, 9H), 0.01 (d, J=2.6 Hz, 6H)

Step 2: To a solution of 2-(4-(benzyloxy)-2-(((tert-butyldimethylsilyl)oxy)methyl)phenyl)-2-(4-bromophenyl)-1-(2,5-difluoro-4-methylphenyl) ethan-1-one (270 mg, 1 eq, 414 μmol) in DCM (2 mL) was added triethylsilane (132 μL, 2 eq, 829 μmol), the resulting mixture was cooled at −78° C. and trimethylsilyl triflate (115 μL, 1.5 eq, 621 μmol) was added dropwise, and the resulting mixture was stirred at −78° C. for 1 h. The reaction was quenched with sat.aq. NaHCO₃ (50 mL) and extracted with EtOAc (30 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 5%) to give the product of 7-(benzyloxy)-4-(4-bromophenyl)-3-(2,5-difluoro-4-methylphenyl)isochromane (120 mg, 50% yield) as a white solid.

¹HNMR (400 MHZ, CDCl₃): δ 7.50-7.34 (m, 5H), 7.19 (d, J=8.3 Hz, 2H), 6.94 (d, J=8.6 Hz, 1H), 6.87-6.79 (m, 2H), 6.77-6.69 (m, 3H), 6.54 (dd, J=6.0, 10.1 Hz, 1H), 5.23 (d, J=3.0 Hz, 1H), 5.19-5.00 (m, 4H), 4.20 (s, 1H), 2.19 (s, 3H)

Step 3: A mixture of 7-(benzyloxy)-4-(4-bromophenyl)-3-(2,5-difluoro-4-methylphenyl) isochromane (120 mg, 1 eq, 230 μmol), 4-(dimethoxymethyl)piperidine (51.3 mg, 1.4 eq, 322 μmol), Xphos (21.9 mg, 0.2 eq, 46.0 μmol), t-BuONa (44.2 mg, 2 eq, 460 μmol) and PdOAc₂ (5.17 mg, 0.1 eq, 23.0 μmol) in Toluene (3 mL) was degassed and purged with N₂ for 3 times, and then the mixture was stirred at 90° C. for 16 h under N₂ atmosphere. The reaction was quenched with water (20 mL) and extracted with EtOAc (20 mL×3). The combined organic layers were washed with brine (30 mL), dried over Na₂SO₄, filtered and concentrated under reduced pressure to give a residue. The residue was purified by flash silica gel chromatography (ethyl acetate in petroleum ether=0% to 20%) to give the product of 1-(4-(7-(benzyloxy)-3-(2,5-difluoro-4-methylphenyl)isochroman-4-yl)phenyl)-4-(dimethoxymethyl)piperidine (80 mg, 49% yield) as a yellow solid.

LC-MS (ESI⁺) m/z: 600.3 (M+H)⁺.

Step 4: To a solution of Pd/C (100 mg, 0.7 eq, 10% Purity) in THF (10 mL) was added 1-(4-(7-(benzyloxy)-3-(2,5-difluoro-4-methylphenyl)isochroman-4-yl)phenyl)-4-(dimethoxymethyl)piperidine (80 mg, 1 eq, 0.13 mmol) under N₂. The suspension was degassed under vacuum and purged with H₂ several times. The mixture was stirred under H₂ (30 psi) at 25° C. for 8 h. The reaction mixture was filtered and the filter was concentrated to give a residue which was purified by preparative HPLC (column: Phenomenex Gemini NX 150×30 mm, 5 μm; mobile phase: [water(NH₄HCO₃)-ACN]; gradient: 60%-90% B over 11 min), followed by lyophilization to yield a residue. The residue was separated by chiral SFC (column: DAICEL CHIRALPAK ID (250 mm*30 mm, 10 um); mobile phase: CO₂-EtOH (0.1% NH₃H₂O) B %: 45%, isocratic elution mode, peak 1:1.269 min, peak 2:2.190 min) to yield Peak 1 and Peak 2. Peak 1 was concentrated under reduced pressure to give the product of (3S,4R)-3-(2,5-difluoro-4-methylphenyl)-4-(4-(4-(dimethoxymethyl)piperidin-1-yl)phenyl)isochroman-7-ol (30 mg, 44% yield, SFC: Rt=1.269) as a white solid. LC-MS (ESI⁺) m/z: 510.3 (M+H)⁺. Peak 2 was concentrated under reduced pressure to give the product of (3R,4S)-3-(2,5-difluoro-4-methylphenyl)-4-(4-(4-(dimethoxymethyl)piperidin-1-yl)phenyl)isochroman-7-ol (30 mg, 44% yield, SFC: R_{f}=2.190) as a white solid. LC-MS (ESI⁺) m/z: 510.3 (M+H)⁺.

Step 5: To a solution of (3S,4R)-3-(2,5-difluoro-4-methylphenyl)-4-(4-(4-(dimethoxymethyl) piperidin-1-yl)phenyl)isochroman-7-ol (30 mg, 1 eq, 59 μmol) in DCM (3 mL) was added TFA (1.0 mL, 230 eq, 14 mmol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give the product of 1-(4-((3S,4R)-3-(2,5-difluoro-4-methylphenyl)-7-hydroxyisochroman-4-yl)phenyl)piperidine-4-carbaldehyde (20 mg, 73% yield) as a yellow solid. LC-MS (ESI⁺) m/z: 464.2 (M+H)⁺.

Step 6: To a solution of (3R,4S)-3-(2,5-difluoro-4-methylphenyl)-4-(4-(4-(dimethoxymethyl) piperidin-1-yl)phenyl)isochroman-7-ol (30 mg, 1 eq, 59 μmol) in DCM (3 mL) was added TFA (1.0 mL, 230 eq, 14 mmol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give the product of 1-(4-((3R,4S)-3-(2,5-difluoro-4-methylphenyl)-7-hydroxyisochroman-4-yl)phenyl)piperidine-4-carbaldehyde (20 mg, 73% yield) as a yellow solid. LC-MS (ESI⁺) m/z: 464.3 (M+H)⁺.

Step 7: To a solution of 1-(4-((3S,4R)-3-(2,5-difluoro-4-methylphenyl)-7-hydroxyisochroman-4-yl)phenyl)piperidine-4-carbaldehyde (30 mg, 1 eq, 65 μmol) in DCE (10 mL) and MeOH (1 mL) was added (S)-3-(1-oxo-5-(piperazin-1-yl)isoindolin-2-yl)piperidine-2,6-dione (21 mg, 1 eq, 65 μmol) and stirred at 20° C. for 10 min. Then sodium triacetoxyhydroborate (41 mg, 3 eq, 0.19 mmol) was added and stirred at 20° C. for 10 min. The solvent was removed to yield a residue. The residue was purified by preparative HPLC (column: Phenomenex Gemini NX 150×30 mm, 5 μm; mobile phase: [water(NH₄HCO₃)-ACN]; gradient: 52%-82% B over 11 min), followed by lyophilization to give the product of (S)-3-(5-(4-((1-(4-((3S,4R)-3-(2,5-difluoro-4-methylphenyl)-7-hydroxyisochroman-4-yl)phenyl)piperidin-4-yl)methyl)piperazin-1-yl)-1-oxoisoindolin-2-yl)piperidine-2,6-dione (11.7 mg, 23% yield) as a white solid. LC-MS (ESI⁺) m/z: 776.3 (M+H)⁺.

LCMS: calc. for C₄₅H₄₇F₂N₅O₅: 775.90, found: [M+H]⁺776.3.

HPLC: 100.0% purity at 254 nm.

¹HNMR (400 MHz, DMSO-d₆): § 10.95 (s, 1H), 9.34 (s, 1H), 7.51 (d, J=8.9 Hz, 1H), 7.15-7.00 (m, 3H), 6.79 (d, J=8.2 Hz, 1H), 6.67-6.51 (m, 6H), 6.43 (dd, J=5.9, 10.3 Hz, 1H), 5.19-5.00 (m, 3H), 4.93 (d, J=15.1 Hz, 1H), 4.40-4.27 (m, 1H), 4.25-4.14 (m, 1H), 3.98 (s, 1H), 3.53 (d, J=11.9 Hz, 2H), 3.27 (s, 6H), 3.00-2.83 (m, 1H), 2.65-2.52 (m, 2H), 2.48 (s, 3H), 2.36 (dq, J=4.4, 13.2 Hz, 1H), 2.17 (d, J=6.9 Hz, 2H), 2.12 (s, 3H), 2.01-1.90 (m, 1H), 1.79-1.55 (m, 3H), 1.14 (q, J=11.8 Hz, 2H)

Step 8: To a solution of 1-(4-((3R,4S)-3-(2,5-difluoro-4-methylphenyl)-7-hydroxyisochroman-4-yl)phenyl)piperidine-4-carbaldehyde (20 mg, 1 eq, 43 μmol) in DCE (10 mL) and MeOH (1 mL) was added (S)-3-(1-oxo-5-(piperazin-1-yl)isoindolin-2-yl)piperidine-2,6-dione (14 mg, 1 eq, 43 μmol) and stirred at 20° C. for 10 min. Then sodium triacetoxyhydroborate (27 mg, 3 eq, 0.13 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 52%-82% B over 11 min), followed by lyophilization to give the product of (S)-3-(5-(4-((1-(4-((3R,4S)-3-(2,5-difluoro-4-methylphenyl)-7-hydroxyisochroman-4-yl)phenyl)piperidin-4-yl)methyl)piperazin-1-yl)-1-oxoisoindolin-2-yl)piperidine-2,6-dione (17.3 mg, 51% yield) as a white solid. LC-MS (ESI⁺) m/z: 776.3 (M+H)⁺.

LCMS: calc. for C₄₅H₄₇F₂N₅O₅: 775.90, found: [M+H]⁺776.3.

HPLC: 100.0% purity at 254 nm.

¹HNMR (400 MHz, DMSO-d₆): § 10.95 (s, 1H), 9.34 (s, 1H), 7.51 (d, J=8.7 Hz, 1H), 7.15-7.00 (m, 3H), 6.79 (d, J=8.2 Hz, 1H), 6.68-6.51 (m, 6H), 6.43 (dd, J=5.8, 10.3 Hz, 1H), 5.14 (d, J=2.7 Hz, 1H), 5.10-5.00 (m, 2H), 4.98-4.88 (m, 1H), 4.38-4.27 (m, 1H), 4.25-4.15 (m, 1H), 3.98 (s, 1H), 3.53 (d, J=12.0 Hz, 2H), 3.27 (s, 6H), 2.97-2.83 (m, 1H), 2.63-2.53 (m, 2H), 2.48 (s, 3H), 2.36 (dq, J=4.6, 13.2 Hz, 1H), 2.17 (d, J=6.9 Hz, 2H), 2.12 (s, 3H), 2.01-1.89 (m, 1H), 1.78-1.57 (m, 3H), 1.22-1.00 (m, 2H).

### Example 124. Preparation of (I-371) (S)-3-(5-(4-((1-(4-((3S,4R)-3-(4-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione and (I-372) (S)-3-(5-(4-((1-(4-((3R,4S)-3-(4-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione

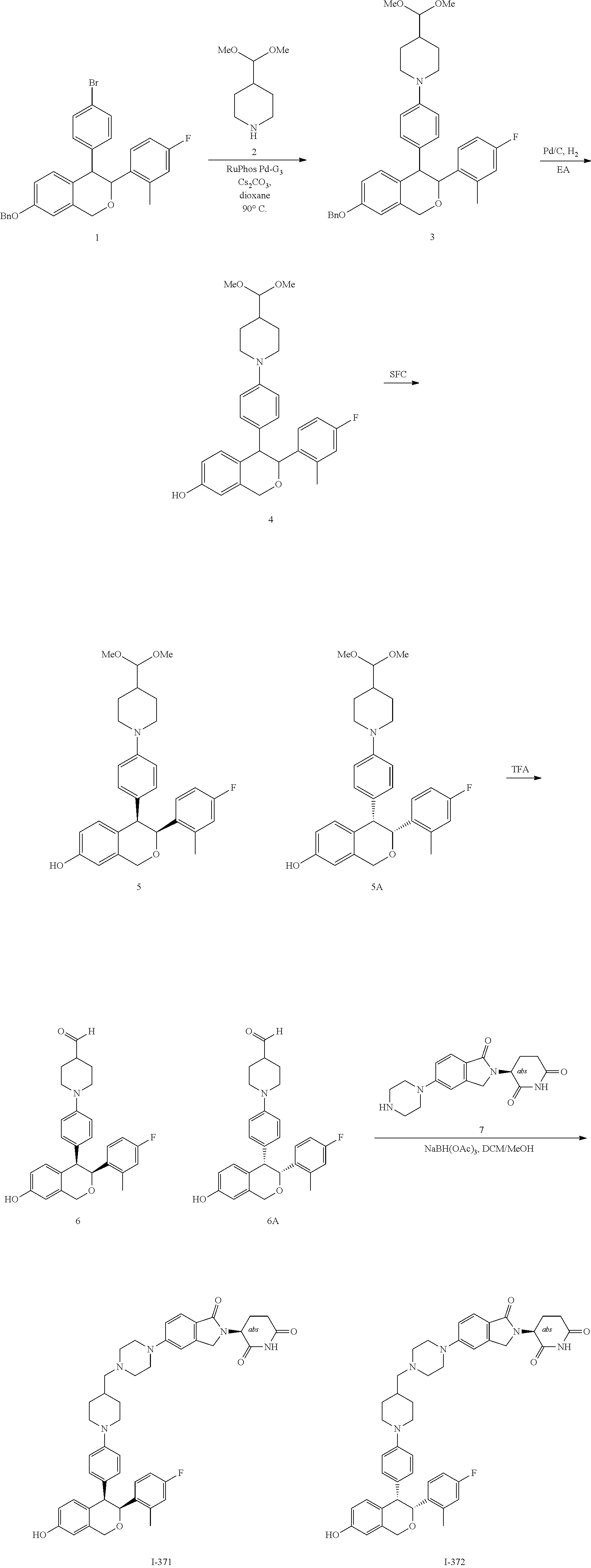

Step 1: To a solution of compound 1 (150 mg, 298 μmol, 1 eq.), compound 2 (94.9 mg, 596 μmol, 2 eq.), Cs₂CO₃ (243 mg, 745 μmol, 2.5 eq.) and RuPhos Pd G3 (24.9 mg, 29.8 μmol, 0.1 eq.) in Dioxane (1.5 mL) was bubbled with N₂ for 1 min. Then the mixture was stirred at 90° C. for 6 hours. LCMS showed the starting material was consumed completely and a major peak with desired MS was detected. The reaction mixture was concentrated to get a residue which was purified by column chromography (SiO₂, Petroleum ether/Ethyl acetate=1/0 to 3/1) to give compound 3 (160 mg, 89% yield) as a yellow solid.

LC-MS (ESI⁺) m/z: 582.3 (M+H)⁺.

¹H NMR (400 MHZ, DMSO-d₆) δ=7.56-7.19 (m, 5H), 6.98-6.79 (m, 4H), 6.64-6.44 (m, 6H), 5.11-5.07 (m, 3H), 5.00-4.92 (m, 1H), 4.05 (d, J=2.9 Hz, 1H), 3.52 (br d, J=11.8 Hz, 2H), 3.27-3.21 (m, 6H), 2.48-2.40 (m, 4H), 2.40-2.34 (m, 3H), 1.70-1.57 (m, 3H), 1.32-1.21 (m, 2H)

Step 2: To a solution of compound 3 (130 mg, 223 μmol, 1 eq.) in Ethyl acetate (10 mL) was added Pd/C (238 mg, 10% Wt, 223 μmol, 1 eq.) under N₂ atmosphere. The suspension was degassed and purged with H₂ for 3 times. The mixture was stirred under H₂ (15 Psi) at 20° C. for 4 hours. LCMS showed the starting material was consumed uncompletely and a major peak with desired MS was detected. The resulting solution was filtrated and concentrated to give a crude, which was purified by prep-HPLC (column: Boston Green ODS 150*30 mm*5 um; mobile phase: [water (TFA)-ACN]; gradient: 22%-52% B over 12 min) to give compound 4 (80 mg, 66% yield) as a white solid.

LC-MS (ESI⁺) m/z: 492.2 (M+H)⁺.

Step 3: Compound 4 (80 mg, 0.16 mmol, 1 eq.) was purified by SFC (Column: DAICEL CHIRALPAK AD 250 mm*30 mm, 10 um, Mobile phase: CO₂-EtOH (0.1% NH₃H₂O), Begin B 50% End B 50% Gradient Time (min) 45, Flow Rate (ml/min) 80, RT: 1.606,2.025) to give compound 5 (20 mg, 25% yield) as a white solid and compound 5A (20 mg, 25% yield) as a white solid.

Compound 5: LC-MS (ESI⁺) m/z: 492.3 (M+H)⁺.

Compound 5A: LC-MS (ESI⁺) m/z: 492.2 (M+H)⁺.

Step 4: To a solution of compound 5 (20 mg, 41 μmol, 1 eq.) in DCM (3 mL) was added TFA (1 mL) at 20° C. The mixture was stirred at 20° C. for 2 hours. LCMS showed the starting material was consumed completely and a major peak with desired MS was detected. Water (10 mL) and aq. NaHCO₃ (10×2 mL) was added and the mixture was extracted with DCM (20 mL×3). The organics were washed with brine (20 mL), dried over Na₂SO₄, filtered and concentrated to give compound 6 (15 mg, 79% yield) as a white solid.

LC-MS (ESI⁺) m/z: 446.4 (M+H)⁺.

Step 5: To a solution of compound 5A (20 mg, 41 μmol, 1 eq.) in DCM (3 mL) was added TEA (1 mL) at 20° C. The mixture was stirred at 20° C. for 2 hours. LCMS showed the starting material was consumed completely and a major peak with desired MS was detected. Water (10 mL) and aq. NaHCO₃ (20×2 mL) was added and the mixture was extracted with DCM (20 mL×3). The organics were washed with brine (20 mL), dried over Na₂SO₄, filtered and concentrated to give compound 6A (16 mg, 85% yield) as a white solid.

LC-MS (ESI⁺) m/z: 446.4 (M+H)⁺.

Step 6: To a solution of compound 6 (15 mg, 34 μmol, 1 eq.) and compound 7 (12 mg, 37 μmol, 1.1 eq) in DCE (3 mL) and MeOH (1 mL) was added NaBH(OAc)₃ (36 mg, 25 μL, 0.17 mmol, 5 eq.) at 20° C. Then the mixture was stirred at 20° C. for 2 hours. LCMS showed the starting material was consumed completely and a major peak with desired MS was detected. The mixture was concentrated to get the crude, which was purified by prep-HPLC (column: Phenomenex Gemini NX 150×30 mm, 5 um; mobile phase: [water(NH₄HCO₃)-ACN]; gradient: 54%-84% B over 11 min) to give 1-371 (13.6 mg, 53% yield) as a white solid.

LC-MS (ESI⁺) m/z: 758.4 (M+H)⁺.

LCMS: calc. for C₄₅H₄₈FN₅O₅: 757.91, found: [M+H]⁺758.4.

HPLC: 100.00% purity at 220 nm

¹H NMR (400 MHZ, DMSO-d₆) δ=10.95 (br s, 1H), 9.31 (s, 1H), 7.51 (d, J=8.6 Hz, 1H), 7.08-7.02 (m, 2H), 6.94 (br d, J=10.4 Hz, 1H), 6.79-6.74 (m, 1H), 6.62-6.54 (m, 6H), 6.52-6.48 (m, 2H), 5.08-5.01 (m, 3H), 4.91 (br d, J=15.4 Hz, 1H), 4.35-4.17 (m, 2H), 4.00 (br s, 1H), 3.51 (br d, J=10.8 Hz, 2H), 3.27 (br s, 6H), 2.94-2.85 (m, 1H), 2.69-2.53 (m, 2H), 2.41 (br d, J=3.6 Hz, 3H), 2.37 (s, 3H), 2.32 (br s, 1H), 2.21-2.14 (m, 2H), 1.99-1.91 (m, 1H), 1.77-1.60 (m, 3H), 1.15 (brd, J=13.0 Hz, 2H)

Step 7: To a solution of compound 6A (20 mg, 45 μmol, 1 eq.) and compound 7 (16 mg, 49 μmol, 1.1 eq.) in DCE (3 mL) and MeOH (1 mL) was added NaBH(OAc)₃ (48 mg, 33 μL, 0.22 mmol, 5 eq.) at 20° C. Then the mixture was stirred at 20° C. for 2 hours. LCMS showed the starting material was consumed completely and a major peak with desired MS was detected. The mixture was concentrated to get the crude, which was purified by prep-HPLC (column:

Phenomenex Gemini NX 150×30 mm, 5 um; mobile phase: [water(NH₄HCO₃)-ACN]; gradient: 49%-79% B over 11 min) to give I-372 (15.5 mg, 46% yield) as a white solid.

LC-MS (ESI⁺) m/z: 758.4 (M+H)⁺.

LCMS: calc. for C₄₅H₄₈FN₅O₅: 757.91, found: [M+H]⁺758.4.

HPLC: 100.00% purity at 220 nm

¹H NMR (400 MHZ, DMSO-d₆) δ=11.01-10.91 (m, 1H), 9.31 (s, 1H), 7.51 (d, J=8.8 Hz, 1H), 7.08-7.02 (m, 2H), 6.94 (br d, J=9.4 Hz, 1H), 6.77 (br d, J=7.9 Hz, 1H), 6.62-6.54 (m, 6H), 6.53-6.47 (m, 2H), 5.09-5.01 (m, 3H), 4.95-4.87 (m, 1H), 4.34-4.17 (m, 2H), 4.00 (br d, J=2.4 Hz, 1H), 3.51 (br d, J=11.1 Hz, 2H), 3.27 (br s, 6H), 2.92-2.86 (m, 1H), 2.61-2.56 (m, 2H), 2.47-2.40 (m, 3H), 2.37 (s, 3H), 2.35-2.31 (m, 1H), 2.18 (br d, J=6.8 Hz, 2H), 1.99-1.93 (m, 1H), 1.77-1.61 (m, 3H), 1.23-1.12 (m, 2H).

EXAMPLE 125, Preparation of (I-373) (S)-3-(5-(4-((1-(4-((3S,4R)-7-hydroxy-3-(3-methoxyphenyl)isochroman-4-yl)phenyl)piperidin-4-yl)methyl)piperazin-1-yl)-1-oxoisoindolin-2-yl)piperidine-2,6-dione and (I-374) (S)-3-(5-(4-((1-(4-((3R,4S)-7-hydroxy-3-(3-methoxyphenyl)isochroman-4-yl)phenyl)piperidin-4-yl)methyl)piperazin-1-yl)-1-oxoisoindolin-2-yl)piperidine-2,6-dione

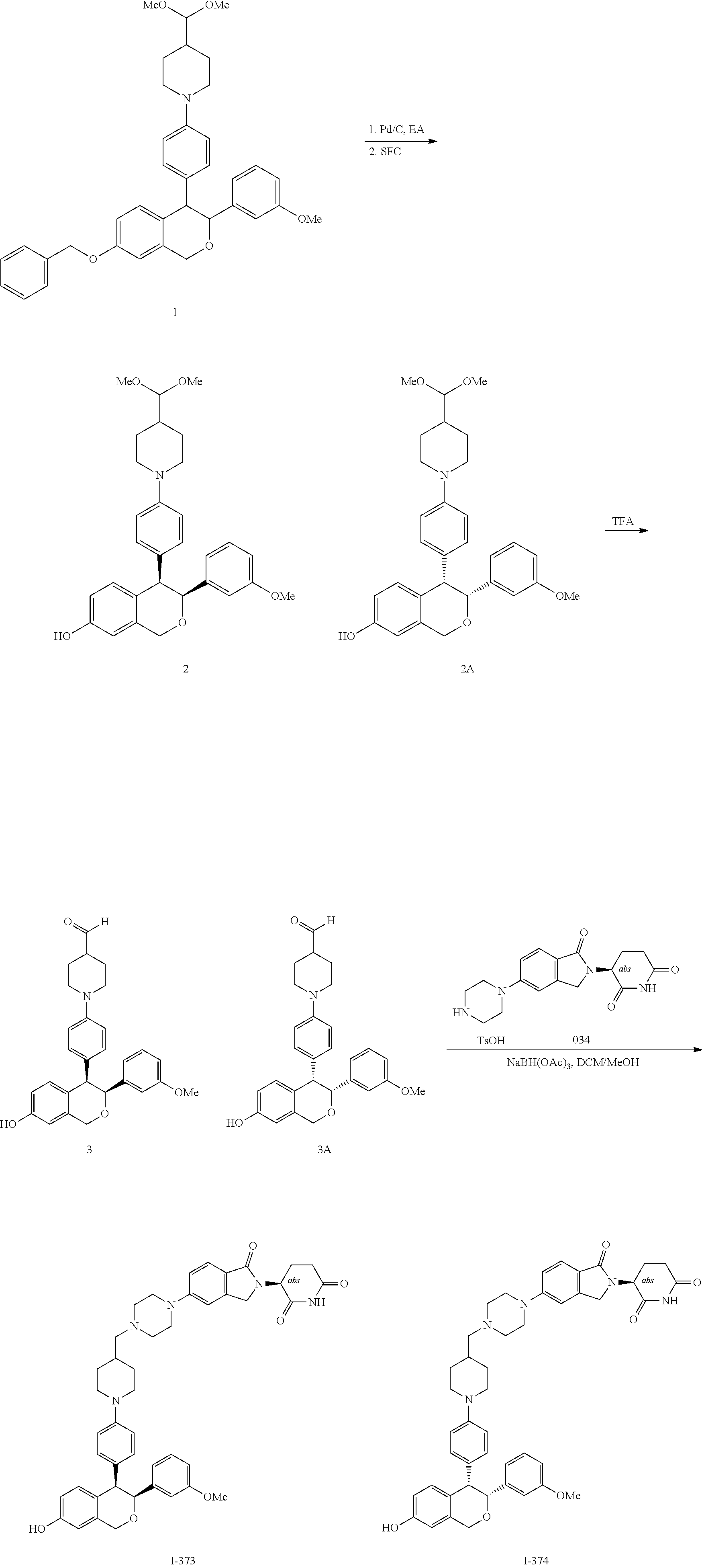

Step 1: A solution of 1 (50 mg, 1 Eq, 86 μmol) and Pd/C (92 mg, 10% Wt, 1 Eq, 86 μmol) in EtOAc (5 mL) and then degassed and purged with H₂ for 3 times, and then the mixture was stirred at 15° C. for 3 hours under H₂ atmosphere. The reaction mixture was filtered and concentrated under reduced pressure to get the product. LCMS showed that the starting material was consumed, and the desired product was detected. The mixture was filtered and concentrated under vacuum to give the product. The product was purified by SFC (condition: CO₂-EtOH (0.1% NH₃H₂O), column: DAICEL CHIRALPAK AD (250 mm×30 mm, 10 um), Begin B: 40, End B: 40) to give 2 (8 mg, 20% yield) as white solid and 2A (9 mg, 20% yield) as white solid. 2: LC-MS (ESI⁺) m/z: 490.2 (M+H)⁺.

2A: LC-MS (ESI⁺) m/z: 490.1 (M+H)⁺.

Step 2: To a solution of 2 (8 mg, 1 Eq, 0.02 mmol) in DCM (1 mL) was added Trifluoroacetic acid (0.4 g, 0.3 mL, 200 Eq, 4 mmol) at 15° C. Then the mixture was stirred at 15° C. for 20 min. LCMS showed that the desired product was detected. Then the mixture was filtered and concentrated under vacuum to give the 3 (10 mg, crude) as red oil.

LC-MS (ESI⁺) m/z: 462.3 (M+H+18)⁺

Step 3: To a solution of 2A (9 mg, 1 Eq, 0.02 mmol) in DCM (1 mL) was added Trifluoroacetic acid (0.4 g, 0.3 mL, 200 Eq, 4 mmol) at 15° C. Then the mixture was stirred at 15° C. for 20 min. LCMS showed that the desired product was detected. Then the mixture was filtered and concentrated under vacuum to give the 3 (10 mg, crude) as red oil.

LC-MS (ESI⁺) m/z: 462.3 (M+H+18)⁺

Step 4: To a solution of 3 (10 mg, 1 Eq, 23 μmol) in DCE (1 mL) and MeOH (1 mL) was added 034 (15 mg, 2 Eq, 45 μmol) and Sodium triacetoxyborohydride (24 mg, 17 μL, 5 Eq. 0.11 mmol) at 15° C. Then the mixture was stirred at 15° C. for 10 min. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 18, 100% B Hold Time (min) 2, Flow Rate (mL/min) 25.) to give I-373 (3.5 mg, 21% yield) as white solid.

LCMS: calc. for C₄₅H₄₉N₅O₆:755.92, found: [M+H]⁺756.5.

HPLC: 100.00% purity at 220 nm

¹H NMR (400 MHZ, DMSO-d₆) δ=10.94 (s, 1H), 9.29 (s, 1H), 8.17 (s, 1H), 7.51 (d, J=8.6 Hz, 1H), 7.11-6.99 (m, 3H), 6.80-6.49 (m, 10H), 5.12-4.85 (m, 4H), 4.35-4.17 (m, 2H), 4.00 (d, J=2.0 Hz, 1H), 3.55 (s, 3H), 3.53-3.48 (m, 2H), 3.27 (br s, 8H), 2.95-2.84 (m, 1H), 2.62-2.52 (m, 3H), 2.39-2.32 (m, 1H), 2.23-2.12 (m, 2H), 2.01-1.91 (m, 1H), 1.74 (br d, J=12.2 Hz, 2H), 1.68-1.57 (m, 1H), 1.21-1.08 (m, 2H).

Step 5: To a solution of 3 (10 mg, 1 Eq, 23 μmol) in DCE (1 mL) and MeOH (1 mL) was added 034 (15 mg, 2 Eq, 45 μmol) and Sodium triacetoxyborohydride (24 mg, 17 μL, 5 Eq, 0.11 mmol) at 15° C. Then the mixture was stirred at 15° C. for 10 min. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 18, 100% B Hold Time (min) 2, Flow Rate (mL/min) 25.) to give 1-374 (3.6 mg, 21% yield) as white solid.

LCMS: calc. for C₄₅H₄₉N₅O₆: 755.92, found: [M+H]⁺756.5.

HPLC: 100.00% purity at 220 nm

¹H NMR (400 MHZ, DMSO-d₆) δ=10.94 (s, 1H), 9.29 (br s, 1H), 8.17 (s, 1H), 7.51 (d, J=8.7 Hz, 1H), 7.09-7.02 (m, 3H), 6.79-6.52 (m, 10H), 5.09-4.87 (m, 4H), 4.35-4.17 (m, 2H), 4.00 (d, J=3.0 Hz, 1H), 3.55 (s, 3H), 3.53-3.48 (m, 2H), 3.27 (br s, 8H), 2.93-2.85 (m, 1H), 2.61-2.53 (m, 3H), 2.38-2.31 (m, 1H), 2.18 (br d, J=6.7 Hz, 2H), 2.00-1.91 (m, 1H), 1.74 (br d, J=12.4 Hz, 2H), 1.68-1.58 (m, 1H), 1.19-1.09 (m, 2H).

### Example 126. Preparation of (I-221) (S)-3-(5-(4-((1-(4-((3S,4S)-7-hydroxy-3-isobutylisochroman-4-yl)phenyl)piperidin-4-yl)methyl)piperazin-1-yl)-1-oxoisoindolin-2-yl)piperidine-2,6-dione and (I-222) (S)-3-(5-(4-((1-(4-((3S,4R)-7-hydroxy-3-isobutylisochroman-4-yl)phenyl)piperidin-4-yl)methyl)piperazin-1-yl)-1-oxoisoindolin-2-yl)piperidine-2,6-dione and (I-223) (S)-3-(5-(4-((1-(4-((3R,4S)-7-hydroxy-3-isobutylisochroman-4-yl)phenyl)piperidin-4-yl)methyl)piperazin-1-yl)-1-oxoisoindolin-2-yl)piperidine-2,6-dione and (I-224) (S)-3-(5-(4-((1-(4-((3R,4R)-7-hydroxy-3-isobutylisochroman-4-yl)phenyl)piperidin-4-yl)methyl)piperazin-1-yl)-1-oxoisoindolin-2-yl)piperidine-2,6-dione

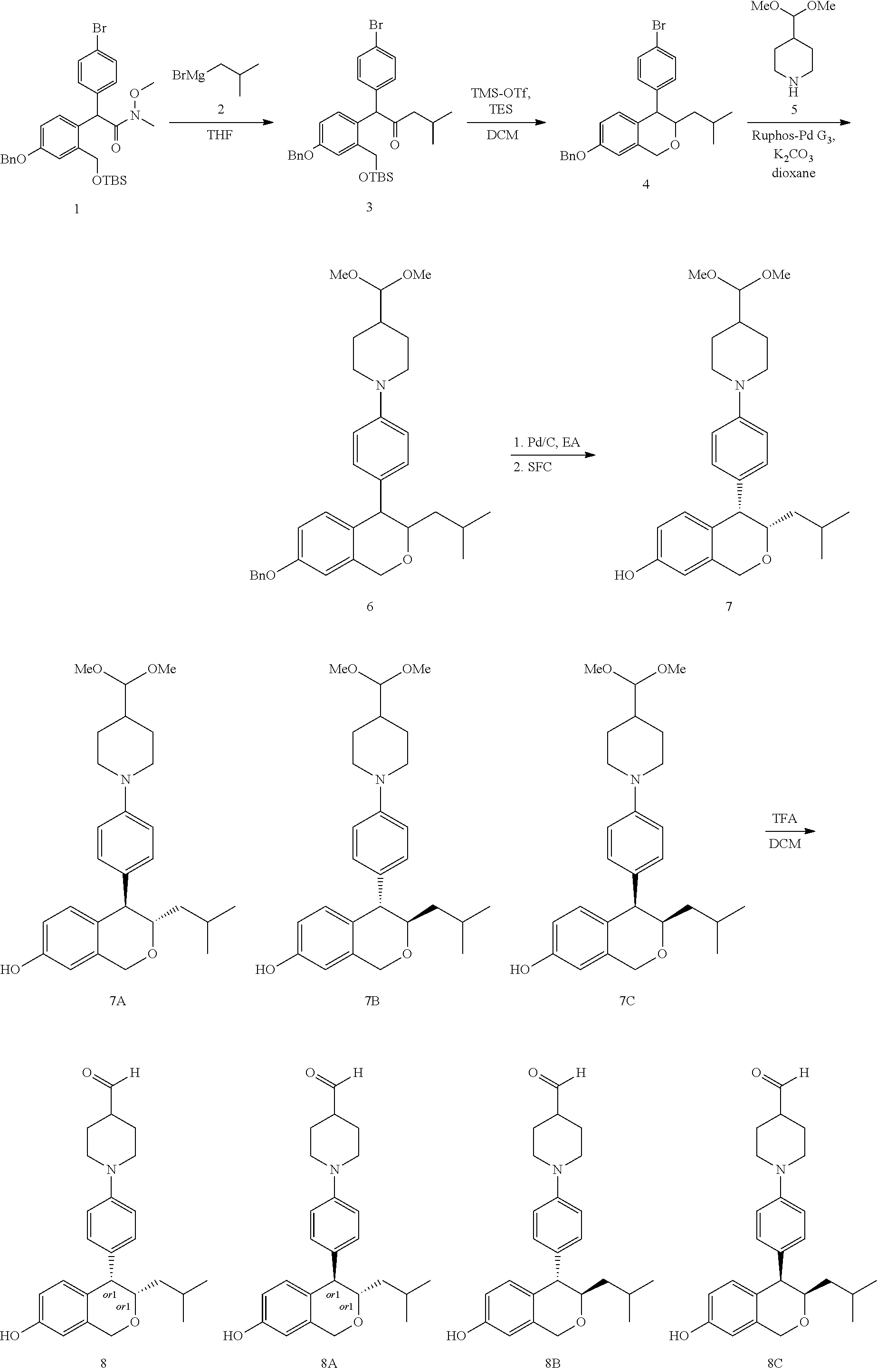

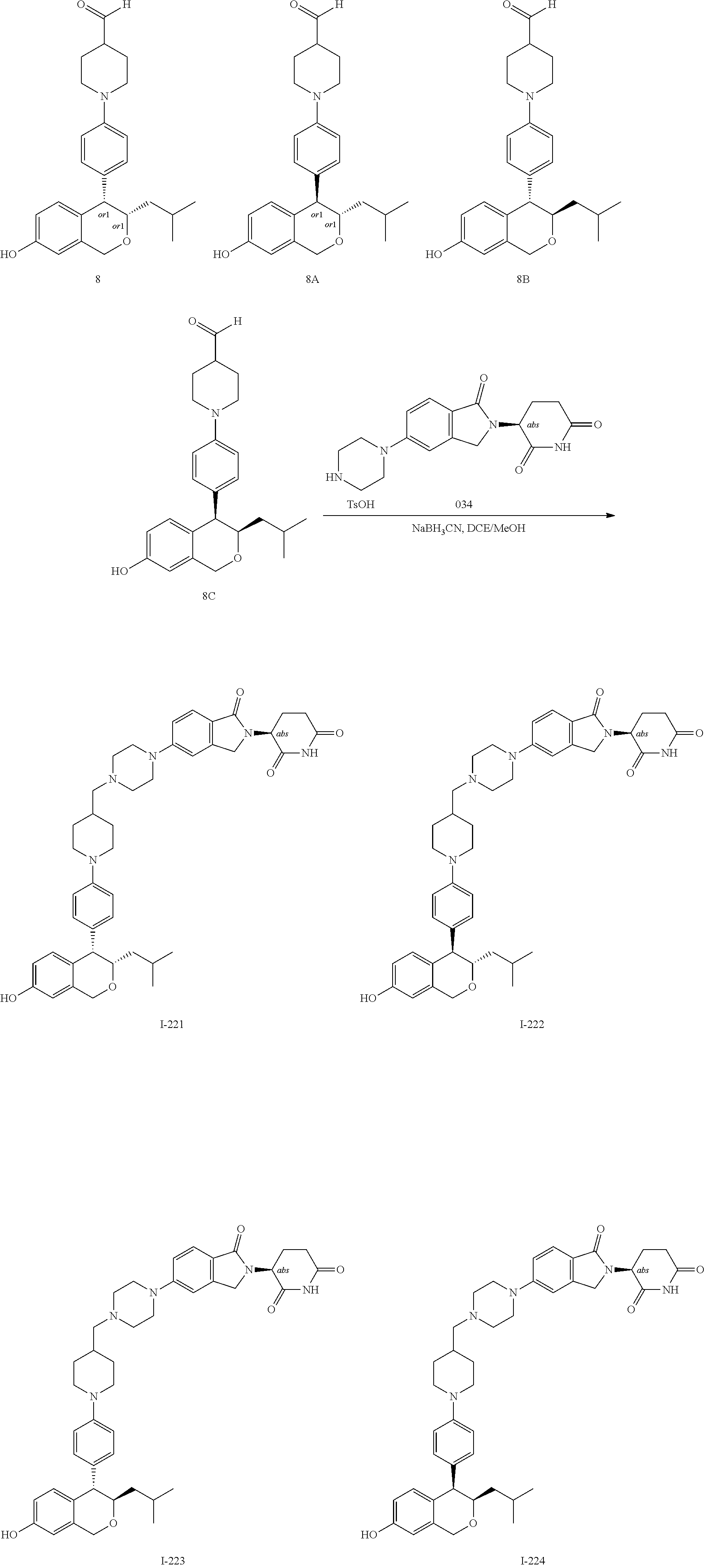

Step 1: To a solution of 3 (1 g, 1 Eq, 2 mmol) in THE (10 mL) was added 2 (1 g, 9 mL, 1 molar, 5 Eq, 9 mmol) at 0° C. under N₂. Then the mixture was stirred at 20° C. for 12 hour under N₂. TLC showed that the new point was detected. NH₄Cl (sat.aq.) (10 mL) was added into the mixture. Then the mixture was filtered and concentrated under vacuum to give the crude. The crude product was purified by flash silica gel chromatography (PE/EA=1/0 to 10/1) to give 3 (250 mg, 30% yield) as colorless oil.

1H NMR (500 MHz, CHLOROFORM-d) δ=7.45-7.32 (m, 7H), 7.08-7.02 (m, 2H), 7.01-6.95 (m, 2H), 6.91-6.86 (m, 1H), 5.36 (s, 1H), 5.07 (s, 2H), 4.71-4.48 (m, 2H), 2.41 (d, J=7.0 Hz, 2H), 2.24-2.12 (m, 1H), 0.93-0.86 (m, 15H), 0.07 (d, J=10.4 Hz, 6H).

Step 2: To a solution of 3 (230 mg, 1 Eq, 395 μmol) in DCM (1 mL) was added Triethylsilane (138 mg, 189 μL, 3 Eq, 1.19 mmol) at 15° C. Then the mixture was cooled to −78° C. The Trimethylsilyl triflate (176 mg, 146 μL, 2 Eq, 791 μmol) was added into the mixture at −78° C. Then the mixture was stirred at −78° C. for 1 hour. TLC showed that the new point was detected. NaHCO₃ (sat.aq.) (5 mL) was added into the mixture. The mixture was purified by Pre-TLC (PE/EA=10/1) give 4 (170 mg, 86% yield) as white solid.

¹H NMR (400 MHZ, CHLOROFORM-d) 8=7.46-7.33 (m, 7H), 7.08-6.99 (m, 2H), 6.90-6.62 (m, 3H), 5.31 (s, 1H), 5.06-5.01 (m, 2H), 5.00-4.85 (m, 2H), 3.96-3.68 (m, 2H), 1.92-1.78 (m, 1H), 1.21-0.93 (m, 2H), 0.89-0.74 (m, 6H).

Step 3: To a solution of 4 (170 mg, 1 Eq, 377 μmol) in 1, 4-dioxane (8 mL) was added 5 (120 mg, 2 Eq, 753 μmol) and Cs₂CO₃ (368 mg, 3 Eq, 1.13 mmol) at 15° C. Then the (2-Dicyclohexylphosphino-2′,6′-diisopropoxy-1,1′-biphenyl) [2-(2′-amino-1,l′-biphenyl)]palladium (II) methanesulfonate (47.2 mg, 0.15 Eq, 56.5 μmol) was added into the mixture at 15° C. The mixture was stirred at 90° C. for 3 hour under N₂. LCMS showed that the starting material was consumed and the desired product was detected. The mixture was filtered and concentrated under vacuum to give the crude. The crude was purified by Pre-TLC (PE/EA=3/1) to give 6 (200 mg, 71.8% yield) as yellow solid.

LC-MS (ESI⁺) m/z: 530.2 (M+H)⁺.

Step 4: A solution of 6 (180 mg, 1 Eq, 340 μmol) and Pd/C (362 mg, 10% Wt, 1 Eq, 340 μmol) in EtOAc (5 mL) and then degassed and purged with H₂ for 3 times, and then the mixture was stirred at 15° C. for 3 hour under H₂ atmosphere. LCMS showed that the starting material was consumed and the desired product was detected. The mixture was filtered and concentrated under vacuum to give the product. The product was purified by SFC (condition: CO₂-EtOH (0.1% NH₃H₂O), column: DAICEL CHIRALPAK IG (250 mm×30 mm, 10 um), Begin B: 45, End B: 45) to give 7 (14.5 mg, 9.63% yield) as white solid, 7A (10 mg, 6.6% yield) as white solid, 7B (10 mg, 6.6% yield) as white solid and 7C (10.5 mg, 6.97% yield) as white solid.

7: LC-MS (ESI⁺) m/z: 440.2 (M+H)⁺.

7A: LC-MS (ESI⁺) m/z: 440.1 (M+H)⁺.

7B: LC-MS (ESI⁺) m/z: 440.2 (M+H)⁺.

7C: LC-MS (ESI⁺) m/z: 440.2 (M+H)⁺.

Step 5: To a solution of 7 (9.5 mg, 1 Eq, 22 μmol) in DCM (1 mL) was added Trifluoroacetic acid (0.4 g, 0.3 mL, 2e+2 Eq, 4 mmol) at 15° C. Then the mixture was stirred at 15° C. for 20 min. LCMS showed that the desired product was detected. Then the mixture was filtered and concentrated under vacuum to give the 8 (10 mg, crude) as red oil.

LC-MS (ESI⁺) m/z: 412.1 (M+H+18)⁺

Step 6: To a solution of 7A (15 mg, 1 Eq, 34 μmol) in DCM (1 mL) was added Trifluoroacetic acid (0.4 g, 0.3 mL, 2e+2 Eq, 4 mmol) at 15° C. Then the mixture was stirred at 15° C. for 20 min. LCMS showed that the desired product was detected. Then the mixture was filtered and concentrated under vacuum to give the 8A (15 mg, crude) as red oil.

LC-MS (ESI⁺) m/z: 412.3 (M+H+18)⁺

Step 7: To a solution of 7B (10 mg, 1 Eq, 23 μmol) in DCM (1 mL) was added Trifluoroacetic acid (0.4 g, 0.3 mL, 2e+2 Eq, 4 mmol) at 15° C. Then the mixture was stirred at 15° C. for 20 min. LCMS showed that the desired product was detected. Then the mixture was filtered and concentrated under vacuum to give the 8B (10 mg, crude) as red oil.

LC-MS (ESI⁺) m/z: 412.2 (M+H+18)⁺

Step 8: To a solution of 7C (10.5 mg, 1 Eq, 23.9 μmol) in DCM (1 mL) was added

Trifluoroacetic acid (0.4 g, 0.3 mL, 2e+2 Eq, 4 mmol) at 15° C. Then the mixture was stirred at 15° C. for 20 min. LCMS showed that the desired product was detected. Then the mixture was filtered and concentrated under vacuum to give the 8C (10 mg, crude) as red oil.

LC-MS (ESI⁺) m/z: 412.2 (M+H+18)⁺

Step 9: To a solution of 8 (10 mg, 1 Eq, 25 μmol) in DCE (1 mL) and MeOH (1 mL) was added 034 (17 mg, 2 Eq, 51 μmol) and Sodium triacetoxyborohydride (27 mg, 19 μL, 5 Eq, 0.13 mmol) at 15° C. Then the mixture was stirred at 15° C. for 0.5 hour. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 18, 100% B Hold Time (min) 2, Flow Rate (mL/min) 25.) to give I-221 (9.8 mg, 55% yield) as a white solid. LC-MS (ESI⁺) m/z: 706.4 (M+H)⁺

LCMS: calc. for C₄₂H₅₁N₅O₅: 705.90, found: [M+H]⁺706.4.

HPLC: 100.00% purity at 220 nm

¹H NMR (400 MHZ, DMSO-d₆) δ=10.95 (s, 1H), 9.19 (s, 1H), 8.13 (s, 1H), 7.66-7.25 (m, 2H), 7.10 (br s, 1H), 6.97-6.82 (m, 4H), 6.50-6.35 (m, 3H), 5.13-5.00 (m, 1H), 4.82-4.66 (m, 2H), 4.37-4.18 (m, 2H), 3.68-3.55 (m, 4H), 3.32-3.20 (m, 8H), 2.95-2.87 (m, 1H), 2.67-2.58 (m, 3H), 2.44-2.11 (m, 3H), 2.00-1.92 (m, 1H), 1.90-1.67 (m, 4H), 1.43-1.35 (m, 1H), 1.31-1.16 (m, 2H), 1.12-1.02 (m, 1H), 0.84-0.68 (m, 6H).

Step 10: To a solution of 8 (15 mg, 1 Eq, 38 μmol) in DCE (1 mL) and MeOH (1 mL) was added 034 (25 mg, 2 Eq, 76 μmol) and Sodium triacetoxyborohydride (40 mg, 5 Eq, 0.19 mmol) at 15° C. Then the mixture was stirred at 15° C. for 0.5 hour. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 18, 100% B Hold Time (min) 2, Flow Rate (mL/min) 25.) to give I-222 (8.1 mg, 30% yield) as a white solid. LC-MS (ESI⁺) m/z: 706.4 (M+H)⁺

LCMS: calc. for C₄₂H₅₁N₅O₅: 705.90, found: [M+H]⁺706.4.

HPLC: 100.00% purity at 220 nm

¹H NMR (400 MHZ, DMSO-d₆) δ=10.95 (s, 1H), 9.19 (s, 1H), 8.13 (s, 1H), 7.65-7.18 (m, 2H), 7.09-7.06 (m, 1H), 6.97-6.83 (m, 4H), 6.50-6.37 (m, 3H), 5.08-5.01 (m, 1H), 4.80-4.67 (m, 2H), 4.35-4.18 (m, 2H), 3.67-3.55 (m, 4H), 3.32-3.12 (m, 8H), 2.93-2.86 (m, 1H), 2.61 (br d, J=10.8 Hz, 3H), 2.43-2.20 (m, 3H), 1.99-1.93 (m, 1H), 1.86-1.67 (m, 4H), 1.45-1.35 (m, 1H), 1.29-1.18 (m, 2H), 1.10-1.02 (m, 1H), 0.82-0.68 (m, 6H).

Step 11: To a solution of 8 (10 mg, 1 Eq, 25 μmol) in DCE (1 mL) and MeOH (1 mL) was added 034 (17 mg, 2 Eq, 51 μmol) and Sodium triacetoxyborohydride (27 mg, 19 μL, 5 Eq, 0.13 mmol) at 15° C. Then the mixture was stirred at 15° C. for 0.5 hour. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 18, 100% B Hold Time (min) 2, Flow Rate (mL/min) 25.) to give 1-223 (11.1 mg, 59% yield) as white solid. LC-MS (ESI⁺) m/z: 706.4 (M+H)⁺

LCMS: calc. for C₄₂H₅₁N₅O₅:705.90, found: [M+H]⁺706.4.

HPLC: 95.27% purity at 220 nm

¹H NMR (400 MHZ, DMSO-d₆) δ=10.95 (s, 1H), 9.20 (s, 1H), 8.13 (s, 1H), 7.70-7.19 (m, 2H), 7.09-7.06 (m, 1H), 6.95 (br d, J=8.6 Hz, 2H), 6.79-6.69 (m, 3H), 6.52-6.43 (m, 2H), 5.08-5.01 (m, 1H), 4.85-4.66 (m, 2H), 4.36-4.18 (m, 2H), 3.81-3.76 (m, 1H), 3.63-3.56 (m, 3H), 3.32-3.18 (m, 8H), 2.93-2.85 (m, 1H), 2.58 (br d, J=13.7 Hz, 3H), 2.44-2.15 (m, 3H), 1.98-1.91 (m, 1H), 1.86-1.67 (m, 4H), 1.27-1.14 (m, 2H), 1.05-0.92 (m, 2H), 0.81 (t, J=6.1 Hz, 6H)

Step 12: To a solution of 8C (10 mg, 1 Eq, 38 μmol) in DCE (1 mL) and MeOH (1 mL) was added 034 (25 mg, 2 Eq, 76 μmol) and Sodium triacetoxyborohydride (40 mg, 19 μL, 5 Eq, 0.19 mmol) at 15° C. Then the mixture was stirred at 15° C. for 0.5 hour. LCMS showed that the desired product was detected. The mixture was filtered and concentrated under vacuum to give the crude. The residue was purified by prep-HPLC (FA condition: Column: Boston Green ODS 150×30 mm×5 um; Condition: water (FA)-ACN, Begin B 10, End B 40, Gradient Time (min) 18, 100% B Hold Time (min) 2, Flow Rate (mL/min) 25.) to give 1-224 (9.2 mg, 50% yield) as a white solid. LC-MS (ESI⁺) m/z: 706.4 (M+H)⁺

LCMS: calc. for C₄₂H₅₁N₅O₅: 705.90, found: [M+H]⁺706.4.

HPLC: 97.36% purity at 220 nm

¹H NMR (400 MHZ, DMSO-d₆) δ=10.95 (s, 1H), 9.20 (s, 1H), 8.13 (s, 1H), 7.67-7.20 (m, 2H), 7.09-7.06 (m, 1H), 6.95 (br d, J=8.6 Hz, 2H), 6.80-6.68 (m, 3H), 6.54-6.41 (m, 2H), 5.10-5.00 (m, 1H), 4.85-4.66 (m, 2H), 4.37-4.18 (m, 2H), 3.82-3.76 (m, 1H), 3.64-3.57 (m, 3H), 3.19 (br d, J=5.4 Hz, 8H), 2.94-2.86 (m, 1H), 2.58 (br d, J=14.5 Hz, 3H), 2.42-2.14 (m, 3H), 1.99-1.92 (m, 1H), 1.85-1.64 (m, 4H), 1.22 (br d, J=10.8 Hz, 2H), 1.04-0.91 (m, 2H), 0.81 (t, J=6.1 Hz, 6H).

### Example 127. Preparation of (I-375) (S)-3-(5-(4-((1-(4-((3S,4R)-3-(4-fluoro-3-methylphenyl)-7-hydroxyisochroman-4-yl)phenyl)piperidin-4-yl)methyl)piperazin-1-yl)-1-oxoisoindolin-2-yl)piperidine-2,6-dione and (I-376) (S)-3-(5-(4-((1-(4-((3R,4S)-3-(4-fluoro-3-methylphenyl)-7-hydroxyisochroman-4-yl)phenyl)piperidin-4-yl)methyl)piperazin-1-yl)-1-oxoisoindolin-2-yl)piperidine-2,6-dione

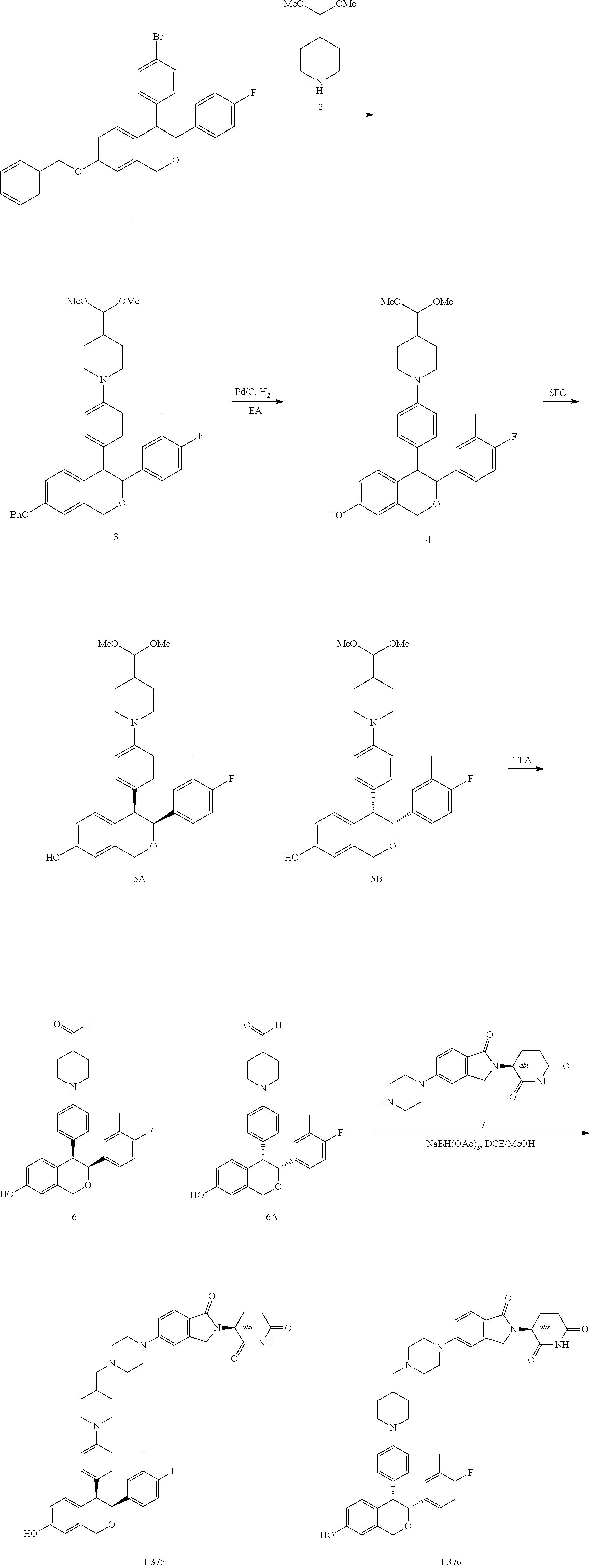

Step 1: A mixture of compound 1 (400.0 mg, 1 eq., 794.6 μmol), 2 (253.0 mg, 2 eq., 1.589 mmol), (2-Dicyclohexylphosphino-2′,6′-diisopropoxy-1,1′-biphenyl) [2-(2′-amino-1,1′-biphenyl)]palladium (II) methanesulfonate (99.69 mg, 0.15 eq., 119.2 μmol) and Cs₂CO₃ (776.7 mg, 3 eq., 2.384 mmol) in Dioxane (20 mL) was degassed and purged with N₂ for 3 times, and then the mixture was stirred at 90° C. for 3 hours under N₂ atmosphere. LCMS showed that the starting material was consumed and the desired product was detected. The reaction mixture was filtered under reduced pressure to give a residue. The residue was poured into water (20 mL). The aqueous phase was extracted with ethyl acetate (40 mL*3). The combined organic phase was washed with brine (20 mL*3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford compound 3 (413 mg, 710 μmol, 89.3%) as yellow oil. The reaction mixture was filtered under reduced pressure to give a compound.

LC-MS (ESI⁺) m/z: 582.4 (M+H)⁺.

Step 2: A mixture of compound 3 (450.0 mg, 1 eq., 773.6 μmol) and Pd/C (493.9 mg, 10% Wt, 0.6 eq., 464.1 μmol) was added in EtOAc (6 mL), and then degassed and purged with H₂ for 3 times, and then the mixture was stirred at 15 psi and 25° C. for 6 hours under H₂ atmosphere. The reaction mixture was filtered and concentrated under reduced pressure to get the product. The residue was purified by prep-HPLC (column: Boston Green ODS 150*30 mm*5 um; mobile phase: [water (TFA)-ACN]; gradient: 29%-59% B over 18 min) to give product. Compound 4 (320.5 mg, 652.0 μmol, 84.28%) was obtained as a white solid.

LC-MS (ESI⁺) m/z: 492.7 (M+H)⁺.

Step 3: Compound 4 (450 mg, 1 eq., 915 μmol) was separated by chiral SFC (Column: DAICELCHIRALPAKAD (250 mm*30 mm*10 um), Condition CO₂-EtOH (0.1% NH₃H₂O) Begin B 55% End B 55%) Flow Rate (ml/min): 110) compound 5A (100 mg, 203 μmol, 22.2%), compound 5B (90 mg, 0.18 mmol, 20%).

Step 4: To a solution of compound 5A (80 mg, 1 eq., 0.16 mmol) in DCM (5 mL) was added TFA (19 mg, 13 μL, 1 eq., 0.16 mmol). The mixture was stirred at 25° C. for 0.5 hr. LCMS showed that the starting material was consumed and the desired product was detected. The reaction liquid was dried with N₂ at 0° C. to give compound 6 (70 mg, 0.16 mmol, 97%) was obtained as yellow oil which was used in the next step without further purification.

LC-MS (ESI+) m/z: 464.3 (M+H)⁺.

Step 5: To a solution of compound 5B (70 mg, 1 eq., 0.14 mmol) in DCM (4 mL) was added TFA (16 mg, 11 μL, 1 eq., 0.14 mmol). The mixture was stirred at 25° C. for 0.2 hr. LCMS showed that the starting material was consumed and the desired product was detected. The reaction liquid is dried with N₂ at 0° C. to give compound 6A (60.5 mg, 136 μmol, 95%) was obtained as yellow oil which was used in the next step without further purification.

LC-MS (ESI⁺) m/z: 464.2 (M+H)⁺.

Step 6: To a solution of compound 6 (90 mg, 1 eq., 0.20 mmol) in DCE (3 mL) and MeOH (2 mL) was added compound 7 (66 mg, 1 eq., 0.20 mmol) at 25° C. After addition, the mixture was stirred at this temperature for 0.5 hr, and then NaBH(OAc)₃ (86 mg, 60 μL, 2 eq., 0.40 mmol) was added at 25° C. The resulting mixture was stirred at 25° C. for 0.5 hr. The reaction mixture was concentrated to yield a residue. The residue was purified by preparative HPLC (column: Boston Green ODS 150*30 mm*5 um; mobile phase: [water (FA)-ACN]; B %: 12%-42%, 18 min), followed by lyophilization to yield I-375 (78.6 mg, 104 μmol, 51%) was obtained as a white solid. LC-MS (ESI⁺) m/z: 758.4 [M+H]⁺.

LCMS: calc. for C₄₅H₄₈FN₅O₅: 757.36, found: [M+H]⁺758.4.

HPLC: 100% purity at 220 nm.

¹H NMR (400 MHZ, DMSO-d₆) δ ppm 10.94 (s, 1H) 9.28 (s, 1H) 8.12 (s, 1H) 7.55 (br d, J=14.78 Hz, 1H) 7.27-7.33 (m, 1H) 7.08 (br s, 1H) 6.84-6.90 (m, 3H) 6.76 (d, J=8.11 Hz, 1H) 6.51-6.60 (m, 6H) 5.00-5.07 (m, 2H) 4.86-4.96 (m, 2H) 4.17-4.34 (m, 2H) 3.86-4.10 (m, 2H) 3.41-3.64 (m, 7H) 2.85-2.93 (m, 1H) 2.51-2.68 (m, 6H) 2.29-2.43 (m, 2H) 2.07 (s, 3H) 1.91-1.98 (m, 1H) 1.63-1.83 (m, 3H) 1.11-1.26 (m, 2H)

Step 7: To a solution of compound 6A (80 mg, 1 eq., 0.18 mmol) in DCE (3 mL) and MeOH (2 mL) was added compound 7 (59 mg, 1 eq., 0.18 mmol) at 25° C. After addition, the mixture was stirred at this temperature for 0.5 hr, and then NaBH(OAc); (76 mg, 53 μL, 2 eq., 0.36 mmol) was added at 25° C. The resulting mixture was stirred at 25° C. for 1 hr. The reaction mixture was concentrated to yield a residue. The residue was purified by preparative HPLC (column: Boston Green ODS 150*30 mm*5 um; mobile phase: [water (FA)-ACN]; B %: 12%-42%, 18 min), followed by lyophilization to yield I-376 (77 mg, 0.10 mmol, 57%) was obtained as a white solid. LC-MS (ESI⁺) m/z: 758.4 [M+H]⁺.

LCMS: calc. for C₄₅H₄₈FN₅O₅: 757.91, found: [M+H]⁺758.4.

HPLC: 98.57% purity at 220 nm

¹H NMR (400 MHZ, DMSO-d₆) δ ppm 10.94 (s, 1H) 9.29 (s, 1H) 8.13 (s, 1H) 7.52-7.60 (m, 1H) 7.15-7.39 (m, 1H) 7.07 (br s, 1H) 6.86-6.91 (m, 3H) 6.78 (d, J=7.99 Hz, 1H) 6.54-6.61 (m, 6H) 5.02-5.08 (m, 2H) 4.87-4.96 (m, 2H) 4.18-4.35 (m, 2H) 3.99 (br s, 1H) 3.52 (br d, J=10.85 Hz, 2H) 3.28 (br d, J=2.15 Hz, 6H) 2.86-2.94 (m, 1H) 2.60 (br s, 4H) 2.30-2.43 (m, 2H) 2.06-2.21 (m, 5H) 1.93-1.99 (m, 1H) 1.61-1.78 (m, 3H) 1.11-1.24 (m, 2H).

### Example 128. Preparation of (I-377) (S)-3-(5-(4-((1-(4-((3S,4R)-3-(3-fluoro-5-methylphenyl)-7-hydroxyisochroman-4-yl)phenyl)piperidin-4-yl)methyl)piperazin-1-yl)-1-oxoisoindolin-2-yl)piperidine-2,6-dione and (I-378) (S)-3-(5-(4-((1-(4-((3R,4S)-3-(3-fluoro-5-methylphenyl)-7-hydroxyisochroman-4-yl)phenyl)piperidin-4-yl)methyl)piperazin-1-yl)-1-oxoisoindolin-2-yl)piperidine-2,6-dione

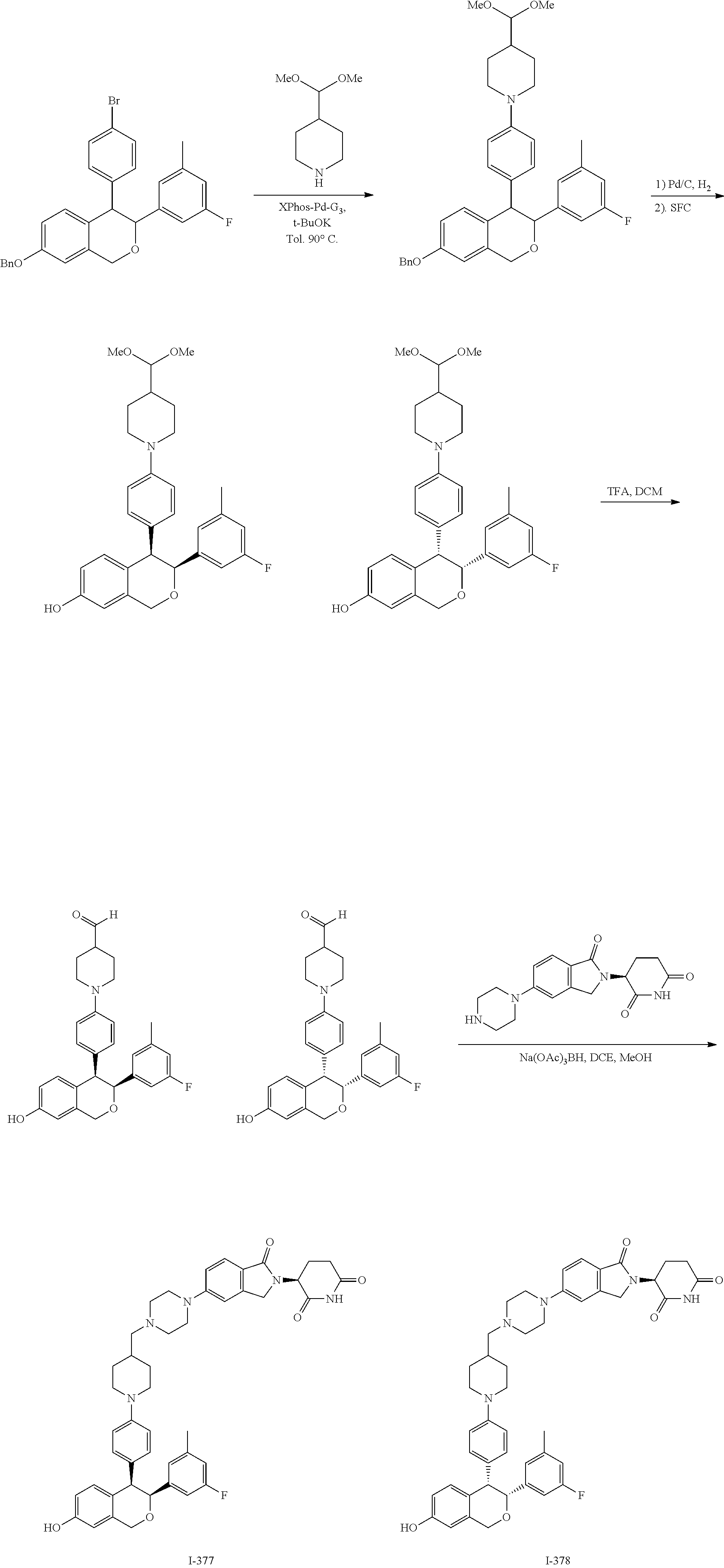

Step 1: A mixture of 7-(benzyloxy)-4-(4-bromophenyl)-3-(3-fluoro-5-methylphenyl)isochromane (240 mg, 477 μmol), 4-(dimethoxymethyl)piperidine (106 mg, 667 μmol), 2-methylpropan-2-olate potassium (134 mg, 1.19 mmol), and XPhos-Pd-G3 (80.7 mg, 95.3 μmol) in Toluene (5 mL) was degassed and purged with N₂ for 3 times, and then the mixture was stirred at 90° C. for 16 h under N₂ atmosphere. The reaction was quenched with water (20 mL) and extracted with EtOAc (20 mL×3). The combined organic layers were washed with brine (30 mL), dried over Na₂SO₄, filtered and concentrated under reduced pressure to give a residue. The residue was purified by flash silica gel chromatography (ethyl acetate in petroleum ether=0% to 20%) to give the product of 1-(4-(7-(benzyloxy)-3-(3-fluoro-5-methylphenyl)isochroman-4-yl)phenyl)-4-(dimethoxymethyl)piperidine (150 mg, 48% yield) as a yellow solid. LC-MS (ESI⁺) m/z: 582.4 (M+H)⁺.

Step 2: To a solution of Pd/C (150 mg, 10% Purity) in THF (10 mL) was added 1-(4-(7-(benzyloxy)-3-(3-fluoro-5-methylphenyl)isochroman-4-yl)phenyl)-4-(dimethoxymethyl)piperidine (150 mg, 258 μmol) under N₂. The suspension was degassed under vacuum and purged with H₂ several times. The mixture was stirred under H₂ (30 psi) at 25° C. for 8 h. The reaction mixture was filtered and the filter was concentrated. The residue was purified by flash silica gel chromatography (ethyl acetate in petroleum ether=0% to 40%) to give a residue which was separated by chiral SFC (column: DAICEL CHIRALPAK ID (250 mm*30 mm, 10 um); mobile phase: CO₂-EtOH (0.1% NH₃H₂O) B %: 45% %, isocratic elution mode, peak 1:1.586 min, peak 2:2.190 min) to yield Peak 1 and Peak 2. Peak 1 was concentrated under reduced pressure to give the product of (3S,4R)-4-(4-(4-(dimethoxymethyl)piperidin-1-yl)phenyl)-3-(3-fluoro-5-methylphenyl)isochroman-7-ol (40 mg, 32% yield, SFC: Rt=1.586) as a white solid. LC-MS (ESI⁺) m/z: 492.3 (M+H)⁺. Peak 2 was concentrated under reduced pressure to yield a product. (3R,4S)-4-(4-(4-(dimethoxymethyl)piperidin-1-yl)phenyl)-3-(3-fluoro-5-methylphenyl)isochroman-7-ol (40 mg, 32% yield, SFC: Rt=2.190) as a white solid. LC-MS (ESI⁺) m/z: 492.4 (M+H)⁺.

Step 3: To a solution of (3S,4R)-4-(4-(4-(dimethoxymethyl)piperidin-1-yl)phenyl)-3-(3-fluoro-5-methylphenyl)isochroman-7-ol (40 mg, 81 μmol) in DCM (3 mL) was added TFA (1 mL, 0.01 mol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give the product of 1-(4-((3S,5S,4R)-3-(3-fluoro-5-methylphenyl)-7-hydroxyisochroman-4-yl)phenyl)piperidine-4-carbaldehyde (30 mg, 75% yield) as a yellow solid. LC-MS (ESI⁺) m/z: 446.3 (M+H)⁺.

Step 4: To a solution of (3R,4S)-4-(4-(4-(dimethoxymethyl)piperidin-1-yl)phenyl)-3-(3-fluoro-5-methylphenyl)isochroman-7-ol (40 mg, 81 μmol) in DCM (3 mL) was added TFA (1 mL, 0.01 mol) at 20° C. The mixture was stirred at 20° C. for 2 h. The reaction was quenched with sat. aq. NaHCO₃ (10 mL) and extracted with EtOAc (10 mL×3). The combined organic layers were washed with brine (20 mL), dried over Na₂SO₄, filtered and concentrated under reduced pressure to give the product of 1-(4-((3R,4S)-3-(3-fluoro-5-methylphenyl)-7-hydroxyisochroman-4-yl)phenyl)piperidine-4-carbaldehyde (30 mg, 77% yield) as a yellow solid. LC-MS (ESI⁺) m/z: 446.3 (M+H)⁺.

Step 5: To a solution of 1-(4-((3S,4R)-3-(3-fluoro-5-methylphenyl)-7-hydroxyisochroman-4-yl)phenyl)piperidine-4-carbaldehyde (30 mg, 67 μmol) in DCE (10 mL) and MeOH (1 mL) was added (S)-3-(1-oxo-5-(piperazin-1-yl)isoindolin-2-yl)piperidine-2,6-dione (22 mg, 67 μmol) and stirred at 20° C. for 10 min. Then sodium triacetoxyhydroborate (43 mg, 0.20 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 48%-78% B over 11 min), followed by lyophilization to give the product of (S)-3-(5-(4-((1-(4-((3S,4R)-3-(3-fluoro-5-methylphenyl)-7-hydroxyisochroman-4-yl)phenyl)piperidin-4-yl)methyl)piperazin-1-yl)-1-oxoisoindolin-2-yl)piperidine-2,6-dione (26.1 mg, 51% yield) as a white solid. LC-MS (ESI⁺) m/z: 758.4 (M+H)⁺

LCMS: calc. for C₄₅H₄₈FN₅O₅: 757.91, found: [M+H]⁺758.4.

HPLC: 99.81% purity at 254 nm.

¹HNMR (400 MHz, DMSO-d₆): § 10.94 (s, 1H), 9.30 (s, 1H), 7.51 (d, J=8.9 Hz, 1H), 7.09-7.00 (m, 2H), 6.81-6.69 (m, 3H), 6.66-6.51 (m, 7H), 5.11-4.99 (m, 2H), 4.96 (d, J=2.7 Hz, 1H), 4.89 (d, J=15.3 Hz, 1H), 4.37-4.16 (m, 2H), 4.02 (d, J=2.6 Hz, 1H), 3.51 (d, J=12.0 Hz, 2H), 3.27 (s, 6H), 3.00-2.83 (m, 1H), 2.63-2.52 (m, 2H), 2.48 (s, 3H), 2.35 (dq, J=4.2, 13.2 Hz, 1H), 2.22-2.13 (m, 5H), 2.00-1.89 (m, 1H), 1.73 (d, J=12.2 Hz, 2H), 1.63 (s, 1H), 1.23-1.06 (m, 2H)

Step 6: To a solution of (3R,4S)-4-(4-(4-(dimethoxymethyl)piperidin-1-yl)phenyl)-3-(3-fluoro-5-methylphenyl)isochroman-7-ol (30 mg, 61 μmol) in DCE (10 mL) and MeOH (1 mL) was added (S)-3-(1-oxo-5-(piperazin-1-yl)isoindolin-2-yl)piperidine-2,6-dione (20 mg, 61 μmol) and stirred at 20° C. for 10 min. Then sodium triacetoxyhydroborate (39 mg, 0.18 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 48%-78% B over 11 min), followed by lyophilization to give the product of (S)-3-(5-(4-((1-(4-((3S,4S)-3-(3-fluoro-5-methylphenyl)-7-hydroxyisochroman-4-yl)phenyl)piperidin-4-yl)methyl)piperazin-1-yl)-1-oxoisoindolin-2-yl)piperidine-2,6-dione (24.9 mg, 54% yield) as a white solid. LC-MS (ESI⁺) m/z: 758.4 (M+H)⁺

LCMS: calc. for C₄₅H₄₈FN₅O₅: 757.91, found: [M+H]⁺758.4.

HPLC: 100.0% purity at 254 nm.

¹HNMR (400 MHz, DMSO-d₆): δ 10.94 (s, 1H), 9.30 (s, 1H), 7.51 (d, J=8.8 Hz, 1H), 7.09-6.99 (m, 2H), 6.81-6.70 (m, 3H), 6.66-6.49 (m, 7H), 5.10-5.00 (m, 2H), 4.99-4.84 (m, 2H), 4.37-4.14 (m, 2H), 4.02 (d, J=2.7 Hz, 1H), 3.51 (d, J=11.9 Hz, 2H), 3.27 (s, 6H), 2.96-2.82 (m, 1H), 2.63-2.51 (m, 2H), 2.48 (s, 3H), 2.36 (dq, J=4.5, 13.3 Hz, 1H), 2.25-2.11 (m, 5H), 2.01-1.90 (m, 1H), 1.73 (d, J=11.6 Hz, 2H), 1.63 (s, 1H), 1.14 (q, J=11.8 Hz, 2H).

### Example 129. Preparation of (I-379) (S)-3-(5-(4-((1-(4-((3S,4R)-3-(3-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione and (I-380) (S)-3-(5-(4-((1-(4-((3R,4S)-3-(3-fluoro-2-methylphenyl)-7-hydroxyisochroman-4-yl)phenyl)piperidin-4-yl)methyl)piperazin-1-yl)-1-oxoisoindolin-2-yl)piperidine-2,6-dione

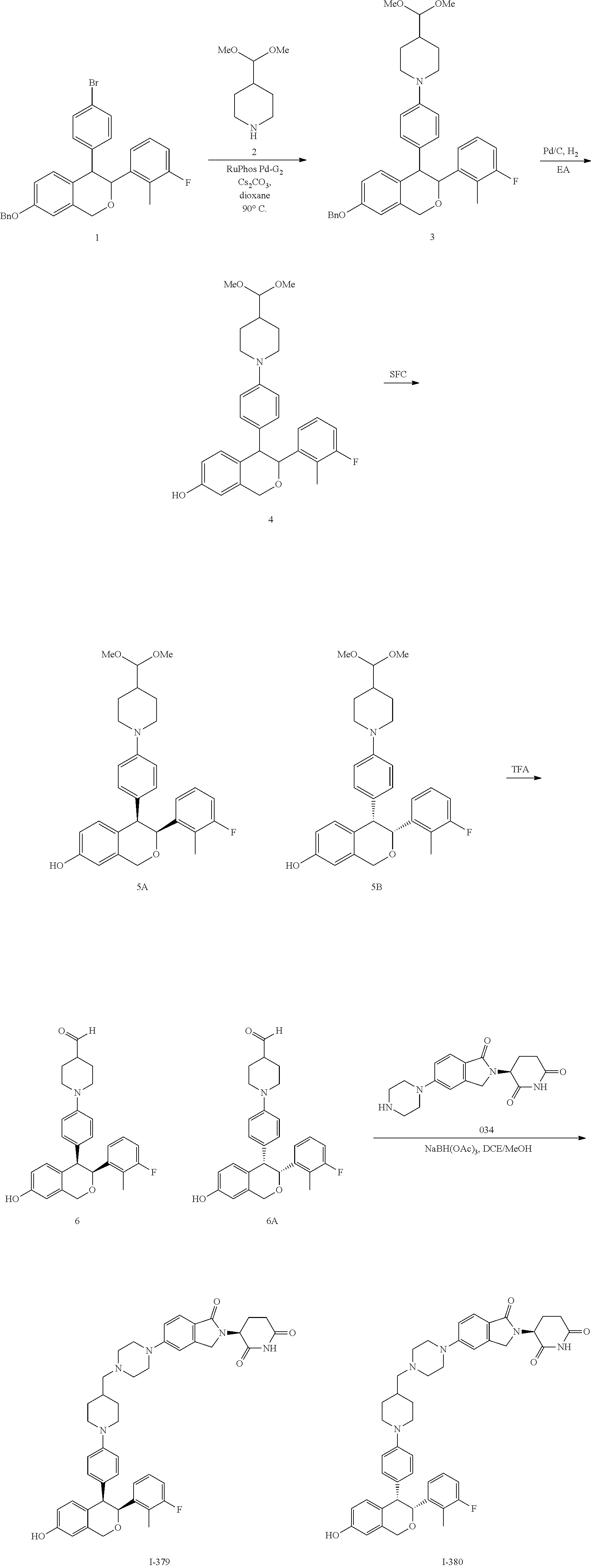

Step 1: A mixture of compound 1 (200.0 mg, 397.3 μmol, 1 eq.), 2 (126.5 mg, 794.6 μmol, 2 eq.), (2-Dicyclohexylphosphino-2′,6′-diisopropoxy-1,1′-biphenyl) [2-(2′-amino-1,l′-biphenyl)]palladium (II) methanesulfonate (49.84 mg, 59.59 μmol, 0.15 eq.) and Cs₂CO₃ (388.3 mg, 1.192 mmol, 3 eq.) in Dioxane (20 mL) was degassed and purged with N₂ for 3 times, and then the mixture was stirred at 90° C. for 3 hour under N₂ atmosphere. LCMS showed that the starting material was consumed and the desired product was detected. The reaction mixture was filtered under reduced pressure to give a residue. The residue was poured into water (20 mL). The aqueous phase was extracted with ethyl acetate (40 mL*3). The combined organic phase was washed with brine (20 mL*3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford compound 3 (173.2 mg, 297.7 μmol, 74.94%) as yellow oil. The reaction mixture was filtered under reduced pressure to give a compound.

LC-MS (ESI⁺) m/z: 582.7 (M+H)⁺.

Step 2: A mixture of compound 3 (115.0 mg, 197.7 μmol, 1 eq.) and Pd/C (126.2 mg, 10% Wt, 118.6 μmol, 0.6 eq.) was added in EtOAc (4 mL), and then degassed and purged with H₂ for 3 times, and then the mixture was stirred at 15 psi and 25° C. for 6 hour under H₂ atmosphere. The reaction mixture was filtered and concentrated under reduced pressure to get the product. The residue was purified by prep-HPLC (column: Boston Green ODS 150*30 mm*5 um; mobile phase: [water (FA)-ACN]; gradient: 25%-55% B over 12 min) to give product. Compound 4 (80 mg, 0.16 mmol, 82%) was obtained as a white solid.

LC-MS (ESI⁺) m/z: 492.3 (M+H)⁺.

Step 3: Compound 4 (0.1 g, 0.2 mmol, 1 eq.) was separated by chiral SFC (Column DAICELCHIRALPAKAD (250 mm*30 mm, 10 um), Condition CO₂-EtOH (0.1% NH₃H₂O) Begin B 40% End B 40%) Flow Rate (ml/min): 150) Compound 5A (35 mg, 71 μmol, 40%), compound 5B (30 mg, 61 μmol, 30%).

Step 4: To a solution of compound 5A (35 mg, 71 μmol, 1 eq.) in DCM (1 mL) was added TFA (0.37 g, 0.25 mL, 3.2 mmol, 46 eq.). The mixture was stirred at 25° C. for 0.5 br. LCMS showed that the starting material was consumed and the desired product was detected. The reaction liquid is dried with N₂ at 0° C. to give compound 6 (30 mg, 67 μmol, 95%) was obtained as yellow oil which was used in the next step without further purification.

LC-MS (ESI⁺) m/z: 464.2 (M+H)⁺.

Step 5: To a solution of compound 5B (30 mg, 61 μmol, 1 eq.) in DCM (1 mL) was added TFA (0.32 g, 0.22 mL, 2.8 mmol, 46 eq.). The mixture was stirred at 25° C. for 0.5 hr. LCMS showed that the starting material was consumed and the desired product was detected. The reaction liquid is dried with N₂ at 0° C. to give Compound 6A (25 mg, 56 μmol, 92%) was obtained as yellow oil which was used in the next step without further purification.

LC-MS (ESI⁺) m/z: 464.3 (M+H)⁺.

Step 6: To a solution of compound 6 (30 mg, 67 μmol, 1 eq.) in DCE (3 mL) and MeOH (2 mL) was added compound 034 (22 mg, 67 μmol, 1 eq.) at 25° C. After addition, the mixture was stirred at this temperature for 0.5 hr, and then NaBH(OAc); (29 mg, 20 μL, 0.13 mmol, 2 eq.) was added at 25° C. The resulting mixture was stirred at 25° C. for 0.5 hr. The reaction mixture was concentrated to yield a residue. The residue was purified by preparative HPLC (column: Phenomenex Gemini NX 150×30 mm, 5 um; mobile phase: [water(NH₄HCO₃)-ACN]; gradient: 49%-79% B over 11 min), followed by lyophilization to yield I-379 (24.1 mg, 31.8 μmol, 47%) was obtained as a white solid. LC-MS (ESI⁺) m/z: 758.4 [M+H]⁺.

LCMS: calc. for C₄₅H₄₅FN₅O₅: 757.91, found: [M+H]⁺758.3.

HPLC: 100% purity at 220 nm.

¹H NMR (400 MHZ, DMSO-d₆) δ ppm 10.94 (br s, 1H) 9.30 (s, 1H) 7.51 (d, J=8.58 Hz, 1H) 7.02-7.08 (m, 2H) 6.76-6.91 (m, 3H) 6.49-6.58 (m, 7H) 5.01-5.13 (m, 3H) 4.89-4.96 (m, 1H) 4.16-4.35 (m, 2H) 4.01 (d, J=2.26 Hz, 1H) 3.50 (br d, J=9.66 Hz, 2H) 3.27 (br s, 4H) 2.86-2.94 (m, 1H) 2.60 (br s, 2H) 2.43-2.49 (m, 5H) 2.31-2.39 (m, 1H) 2.16-2.27 (m, 5H) 1.92-1.99 (m, 1H) 1.60-1.78 (m, 3H) 1.09-1.21 (m, 2H)

Step 7: To a solution of compound 6A (25 mg, 56 μmol, 1 eq.) in DCE (3 mL) and MeOH (2 mL) was added compound 034 (18 mg, 56 μmol, 1 eq.) at 25° C. After addition, the mixture was stirred at this temperature for 0.5 hr and then NaBH(OAc)₃ (24 mg, 17 μL, 0.11 mmol, 2 eq.) was added at 25° C. The resulting mixture was stirred at 25° C. for 1 hr. The reaction mixture was concentrated to yield a residue. The residue was purified by preparative HPLC (column: Phenomenex Gemini NX 150×30 mm, 5 um; mobile phase: [water(NH₄HCO₃)-ACN]; gradient: 49%-79% B over 11 min), followed by lyophilization to yield I-380 (21.9 mg, 28.9 μmol, 51%) was obtained as a white solid. LC-MS (ESI⁺) m/z: 758.4 [M+H]⁺.

LCMS: calc. for C₄₅H₄₈FN₅O₅: 757.91, found: [M+H]⁺758.4.

HPLC: 100% purity at 220 nm

¹H NMR (400 MHZ, DMSO-d₆) δ ppm 10.88-11.00 (m, 1H) 9.24-9.39 (m, 1H) 7.52 (d, J=8.58 Hz, 1H) 7.03-7.08 (m, 2H) 6.77-6.91 (m, 3H) 6.51-6.59 (m, 7H) 5.13 (d, J=2.86 Hz, 1H) 5.02-5.09 (m, 2H) 4.90-4.97 (m, 1H) 4.29-4.36 (m, 1H) 4.16-4.25 (m, 1H) 4.02 (d, J=2.50 Hz, 1H) 3.50 (br d, J=10.49 Hz, 2H) 3.28 (br s, 4H) 2.86-2.95 (m, 1H) 2.53-2.75 (m, 2H) 2.42 (br s, 5H) 2.32-2.39 (m, 1H) 2.26 (d, J=1.31 Hz, 3H) 2.19 (br d, J=6.91 Hz, 2H) 1.93-2.00 (m, 1H) 1.60-1.77 (m, 3H) 1.12-1.24 (m, 2H).

### Example 130. Preparation of (I-242) (S)-3-(5-(4-((1-(4-((3S,4S)-3-cyclopropyl-7-hydroxyisochroman-4-yl)phenyl)piperidin-4-yl)methyl)piperazin-1-yl)-1-oxoisoindolin-2-yl)piperidine-2,6-dione and (I-243) (S)-3-(5-(4-((1-(4-((3R,4R)-3-cyclopropyl-7-hydroxyisochroman-4-yl)phenyl)piperidin-4-yl)methyl)piperazin-1-yl)-1-oxoisoindolin-2-yl)piperidine-2,6-dione

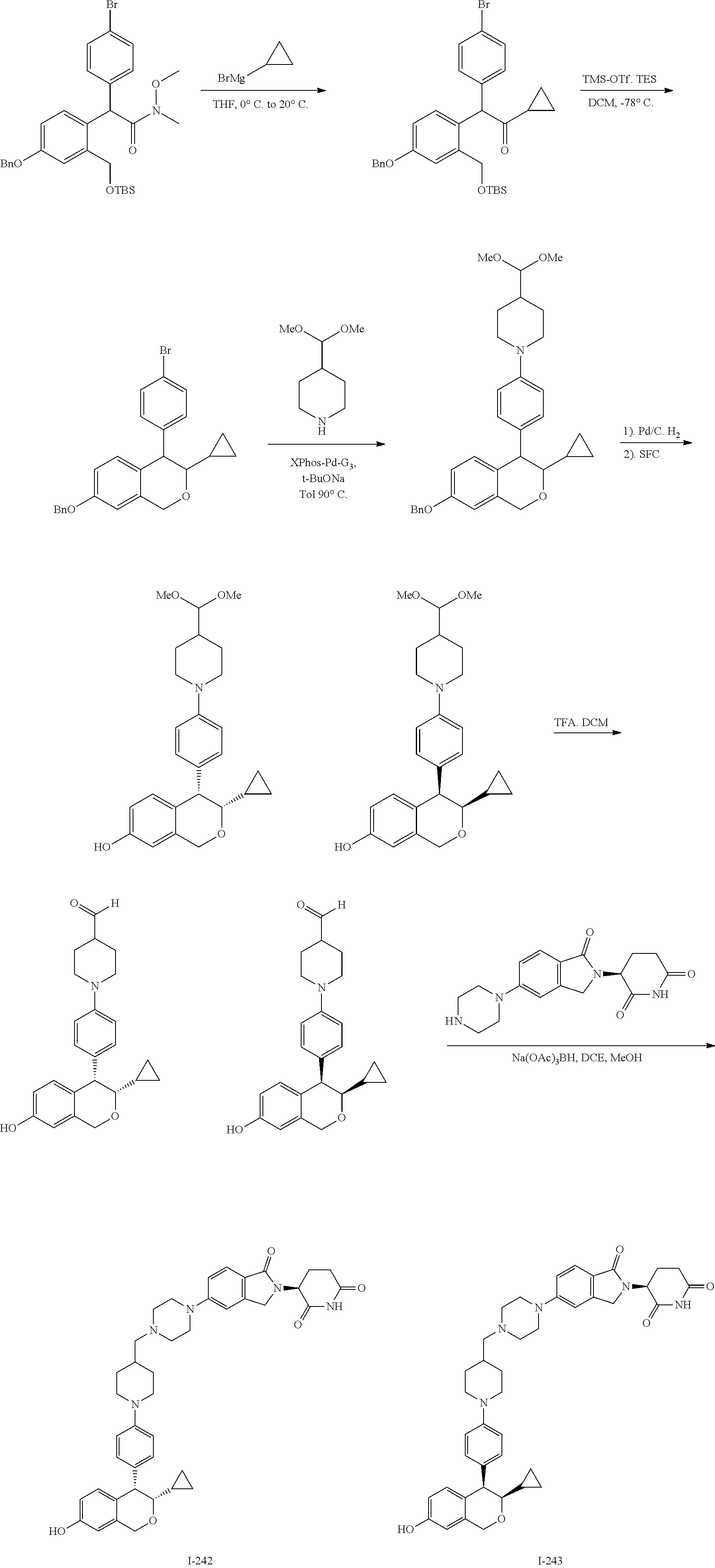

Step 1: To a solution of 2-(4-(benzyloxy)-2-(((tert-butyldimethylsilyl)oxy)methyl)phenyl)-2-(4-bromophenyl)-N-methoxy-N-methylacetamide (1 g, 2 mmol) in THF (10 mL) was added cyclopropylmagnesium bromide solution (20 mL, 0.5M, 10 mmol) dropwise at 0° C. and stirred at 20° C. for 16 h under N₂ atmosphere. The reaction was quenched with sat.aq. NaHCO₃ (50 mL) and extracted with EtOAc (30 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 5%) to give a product of 2-(4-(benzyloxy)-2-(((tert-butyldimethylsilyl)oxy)methyl)phenyl)-2-(4-bromophenyl)-1-cyclopropylethan-1-one (850 mg, 80% yield) as a white solid 1HNMR (400 MHz, CDCl₃): $7.49-7.30 (m, 8H), 7.09 (d, J=2.6 Hz, 1H), 7.02-6.98 (m, 2H), 6.89 (dd, J=2.7, 8.5 Hz, 1H), 5.54 (s, 1H), 5.07 (s, 2H), 4.71 (d, J=12.8 Hz, 1H), 4.53 (d, J=12.8 Hz, 1H), 2.02-1.94 (m, 1H), 1.16-1.03 (m, 2H), 0.92 (s, 9H), 0.89-0.82 (m, 2H), 0.07 (d, J=8.9 Hz, 6H)

Step 2: To a solution of 2-(4-(benzyloxy)-2-(((tert-butyldimethylsilyl)oxy)methyl)phenyl)-2-(4-bromophenyl)-1-cyclopropylethan-1-one (850 mg, 1.50 mmol) in DCM (10 mL) was added triethylsilane (480 μL, 3.01 mmol), the resulting mixture was cooled at −78° C. and trimethylsilyl triflate (416 μL, 2.25 mmol) was added dropwise, and the resulting mixture was stirred at −78° C. for 1 h. The reaction was quenched with sat.aq. NaHCO₃ (50 mL) and extracted with EtOAc (30 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 7%) to give the product of 7-(benzyloxy)-4-(4-bromophenyl)-3-cyclopropylisochromane (500 mg, 69% yield) as a white solid.

¹HNMR (400 MHZ, CDCl₃): δ 7.45-7.34 (m, 7H), 7.11 (d, J=8.3 Hz, 2H), 6.90 (d, J=8.6 Hz, 1H), 6.79 (dd, J=2.5, 8.5 Hz, 1H), 6.71-6.62 (m, 1H), 5.05-4.98 (m, 3H), 4.88-4.77 (m, 1H), 3.89 (d, J=3.1 Hz, 1H), 3.02 (dd, J=3.3, 8.0 Hz, 1H), 0.56-0.39 (m, 4H), 0.30-0.22 (m, 1H)

Step 3: A mixture of 7-(benzyloxy)-4-(4-bromophenyl)-3-cyclopropylisochromane (500 mg, 1.15 mmol), 4-(dimethoxymethyl)piperidine (256 mg, 1.61 mmol), XPhos-Pd-G3 (97.2 mg, 115 μmol), and t-BuONa (221 mg, 2.30 mmol) in 1,4-Dioxane (10 mL) was degassed and purged with N₂ for 3 times, and then the mixture was stirred at 90° C. for 16 h under N₂ atmosphere. The reaction was quenched with water (20 mL) and extracted with EtOAc (20 mL×3). The combined organic layers were washed with brine (30 mL), dried over Na₂SO₄, filtered and concentrated under reduced pressure to give a residue. The residue was purified by flash silica gel chromatography (ethyl acetate in petroleum ether=0% to 20%) to give the product of 1-(4-(7-(benzyloxy)-3-cyclopropylisochroman-4-yl)phenyl)-4-(dimethoxymethyl)piperidine (250 mg, 39% yield) as a white solid. LC-MS (ESI⁺) m/z: 514.4 (M+H)⁺.

Step 4: To a solution of Pd/C (200 mg, 10% Purity) in THF (10 mL) was added 1-(4-(7-(benzyloxy)-3-cyclopropylisochroman-4-yl)phenyl)-4-(dimethoxymethyl)piperidine (250 mg, 487 μmol) under N₂. The suspension was degassed under vacuum and purged with H₂ several times. The mixture was stirred under H₂ (50 psi) at 25° C. for 6 h. The reaction mixture was filtered and the filter was concentrated to give a residue. The residue was separated by chiral SFC (column: DAICEL CHIRALCEL OD-H (250 mm*30 mm, 5 um); mobile phase: CO₂-MeOH (0.1% NH₃H₂O); B %: 40%, isocratic elution mode, peak 1:1.830 min, peak 2:2.433 min) to yield Peak 1 and Peak 2. Peak 1 was concentrated under reduced pressure to give the product of (3S,4S)-3-cyclopropyl-4-(4-(4-(dimethoxymethyl)piperidin-1-yl)phenyl)isochroman-7-ol (60 mg, 29% yield, SFC: R_{f}=1.825) as a white solid. LC-MS (ESI⁺) m/z: 424.2 (M+H)⁺. Peak 2 was concentrated under reduced pressure to give the product of (3R,4R)-3-cyclopropyl-4-(4-(4-(dimethoxymethyl)piperidin-1-yl)phenyl)isochroman-7-ol (60 mg, 29% yield, SFC: Rt=2.418) as a white solid. LC-MS (ESI⁺) m/z: 424.2 (M+H)⁺.

Step 5: To a solution of (3S,4S)-3-cyclopropyl-4-(4-(4-(dimethoxymethyl)piperidin-1-yl)phenyl)isochroman-7-ol (60 mg, 0.14 mmol) in DCM (3 mL) was added TFA (1 mL, 0.01 mol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give the product of 1-(4-((3S,4S)-3-cyclopropyl-7-hydroxyisochroman-4-yl)phenyl)piperidine-4-carbaldehyde (50 mg, 94% yield) as a yellow solid. LC-MS (ESI⁺) m/z: 378.3 (M+H)⁺.

Step 6: To a solution of (3R,4R)-3-cyclopropyl-4-(4-(4-(dimethoxymethyl)piperidin-1-yl)phenyl)isochroman-7-ol (60 mg, 0.14 mmol) in DCM (3 mL) was added TFA (1 mL, 0.01 mol) at 20° C. The mixture was stirred at 20° C. for 2 h. The reaction was quenched with sat.aq. NaHCO₃ (10 mL) and extracted with EtOAc (10 mL×3). The combined organic layers were washed with brine (20 mL), dried over Na₂SO₄, filtered and concentrated under reduced pressure to give the product of 1-(4-((3R,4R)-3-cyclopropyl-7-hydroxyisochroman-4-yl)phenyl)piperidine-4-carbaldehyde (50 mg, 94% yield) as a yellow solid. LC-MS (ESI⁺) m/z: 378.3 (M+H)⁺

Step 7: To a solution of 1-(4-((3S,4S)-3-cyclopropyl-7-hydroxyisochroman-4-yl)phenyl)piperidine-4-carbaldehyde (50 mg, 0.13 mmol) in DCE (10 mL) and MeOH (1 mL) was added (S)-3-(1-oxo-5-(piperazin-1-yl)isoindolin-2-yl)piperidine-2,6-dione (43 mg, 0.13 mmol) and stirred at 20° C. for 10 min. Then sodium triacetoxyhydroborate (84 mg, 0.40 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 43%-73% B over 11 min), followed by lyophilization to give the product of (S)-3-(5-(4-((1-(4-((3S,4S)-3-cyclopropyl-7-hydroxyisochroman-4-yl)phenyl)piperidin-4-yl)methyl)piperazin-1-yl)-1-oxoisoindolin-2-yl)piperidine-2,6-dione (50.4 mg, 55% yield) as a white solid. LC-MS (ESI⁺) m/z: 690.3 (M+H)⁺

LCMS: calc. for C41H₄₇N₅O₅: 689.86, found: [M+H]⁺690.3.

HPLC: 100.0% purity at 254 nm.

¹HNMR (500 MHz, DMSO-d₆): δ 10.93 (s, 1H), 9.20 (s, 1H), 7.52 (d, J=8.4 Hz, 1H), 7.11-7.03 (m, 2H), 6.98 (d, J=8.7 Hz, 2H), 6.78 (d, J=8.7 Hz, 2H), 6.73 (d, J=8.4 Hz, 1H), 6.51 (dd, J=2.4, 8.3 Hz, 1H), 6.45 (d, J=2.1 Hz, 1H), 5.04 (dd, J=5.1, 13.4 Hz, 1H), 4.83 (d, J=15.4 Hz, 1H), 4.63 (d, J=15.3 Hz, 1H), 4.37-4.28 (m, 1H), 4.25-4.15 (m, 1H), 3.74 (d, J=2.7 Hz, 1H), 3.66-3.54 (m, 2H), 3.31-3.22 (m, 7H), 2.98-2.83 (m, 2H), 2.63-2.52 (m, 4H), 2.40-2.34 (m, 1H), 2.21 (d, J=7.2 Hz, 2H), 2.00-1.91 (m, 1H), 1.79 (d, J=11.6 Hz, 2H), 1.72-1.59 (m, 1H), 1.28-1.12 (m, 2H), 0.38-0.30 (m, 3H), 0.29-0.23 (m, 2H)

Step 8: To a solution of 1-(4-(3R,4R)-3-cyclopropyl-7-hydroxyisochroman-4-yl)phenyl)piperidine-4-carbaldehyde (50 mg, 0.13 mmol) in DCE (10 mL) and MeOH (1 mL) was added (S)-3-(1-oxo-5-(piperazin-1-yl)isoindolin-2-yl)piperidine-2,6-dione (43 mg, 0.13 mmol) and stirred at 20° C. for 10 min. Then sodium triacetoxyhydroborate (84 mg, 0.40 mmol) was added and stirred at 20° C. for 10 min. The solvent was removed to yield a residue. The residue was purified by preparative HPLC (column: Phenomenex Gemini NX 150×30 mm, 5 um; mobile phase: [water(NH₄HCO₃)-ACN]; gradient: 43%-73% B over 11 min), followed by lyophilization to give the product of (S)-3-(5-(4-((1-(4-((3R,4R)-3-cyclopropyl-7-hydroxyisochroman-4-yl)phenyl)piperidin-4-yl)methyl)piperazin-1-yl)-1-oxoisoindolin-2-yl)piperidine-2,6-dione (45 mg, 49% yield) as a white solid. LC-MS (ESI⁺) m/z: 690.3 (M+H)⁺.

LCMS: calc. for C₄₁H₄₇N₅O₅:689.86, found: [M+H]⁺690.3.

HPLC: 100.0% purity at 254 nm.

¹HNMR (500 MHz, DMSO-d₆): § 10.94 (s, 1H), 9.20 (s, 1H), 7.52 (d, J=8.5 Hz, 1H), 7.12-7.02 (m, 2H), 6.98 (d, J=8.7 Hz, 2H), 6.78 (d, J=8.9 Hz, 2H), 6.73 (d, J=8.4 Hz, 1H), 6.51 (dd, J=2.4, 8.3 Hz, 1H), 6.44 (d, J=2.1 Hz, 1H), 5.04 (dd, J=5.2, 13.3 Hz, 1H), 4.83 (d, J=15.3 Hz, 1H), 4.63 (d, J=15.3 Hz, 1H), 4.36-4.29 (m, 1H), 4.24-4.17 (m, 1H), 3.74 (d, J=2.7 Hz, 1H), 3.65-3.55 (m, 2H), 3.30-3.26 (m, 7H), 2.97-2.84 (m, 2H), 2.62-2.54 (m, 4H), 2.39-2.34 (m, 1H), 2.21 (d, J=7.2 Hz, 2H), 2.00-1.91 (m, 1H), 1.79 (d, J=11.7 Hz, 2H), 1.72-1.60 (m, 1H), 1.27-1.12 (m, 2H), 0.37-0.31 (m, 3H), 0.28-0.23 (m, 2H).

### Example 131. Preparation of (I-381) (S)-3-(5-(4-((1-(2-fluoro-4-((3S,4R)-7-hydroxy-3-phenylisochroman-4-yl)phenyl)piperidin-4-yl)methyl)piperazin-1-yl)-1-oxoisoindolin-2-yl)piperidine-2,6-dione and (I-382) (S)-3-(5-(4-((1-(2-fluoro-4-((3R,4S)-7-hydroxy-3-phenylisochroman-4-yl)phenyl)piperidin-4-yl)methyl)piperazin-1-yl)-1-oxoisoindolin-2-yl)piperidine-2,6-dione

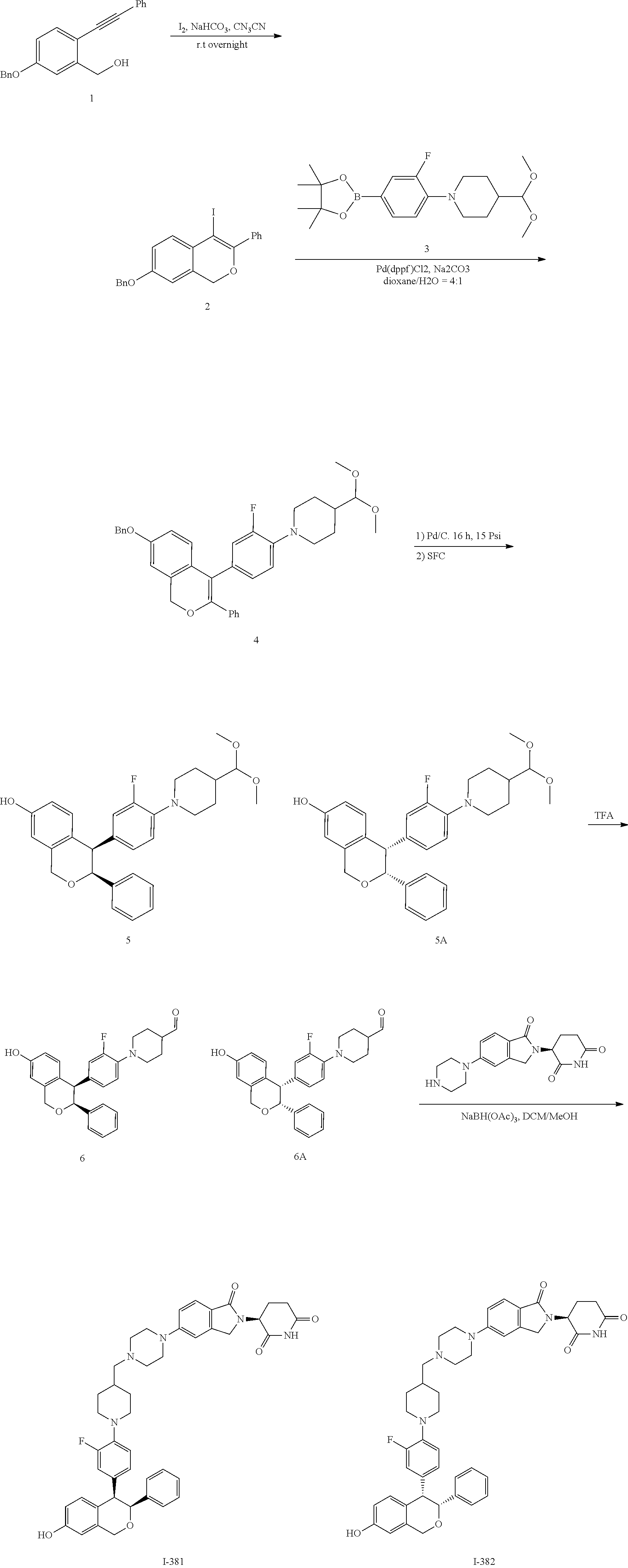

Step 1: To a solution of (5-(benzyloxy)-2-(phenylethynyl)phenyl) methanol (1 g, 3 mmol, 1 eq.) and Sodium bicarbonate (0.8 g, 0.4 mL, 0.01 mol, 3 eq.) in MeCN (100 mL) was added I₂ (2 g, 0.01 mol, 3 eq.). The mixture was stirred at 25° C. for 12 h. The reaction mixture was quenched by addition of Na₂S₂O₃ (50 mL, 10%), extracted with EtOAc (30 mL×3). The combined organic layers were washed with brine (10 mL), dried over Na₂SO₄, filtered and concentrated under reduced pressure to give a residue. The residue was purified by flash silica gel chromatography (from PE/EtOAc=1/0 to 20/1, TLC: PE/EtOAc=10/1, R_{f}=0.49) to yield 7-(benzyloxy)-4-iodo-3-phenyl-1H-isochromene (370 mg, 0.76 mmol, 20%, 90% Purity) as a yellow solid. LC-MS (ESI+) m/z: 439.8 (M−1)⁺

¹H NMR (400 MHz, CDCl₃) δ=7.65 (dd, J=2.4, 7.3 Hz, 2H), 7.49-7.31 (m, 9H), 6.94 (dd, J=2.6, 8.5 Hz, 1H), 6.69 (d, J=2.5 Hz, 1H), 5.19 (s, 2H), 5.11 (s, 2H).

Step 2: To a solution of 7-(benzyloxy)-4-iodo-3-phenyl-1H-isochromene (500 mg, 1.14 mmol, 1 eq.) and 4-(dimethoxymethyl)-1-(2-fluoro-4-(4,4,5,5-tetramethyl-1,3,2-dioxaborolan-2-yl)phenyl)piperidine (574 mg, 75% Wt, 1.14 mmol, 1 eq.) in 1,4-Dioxane (8 mL) and H₂O (2 mL) was added sodium carbonate (301 mg, 2.84 mmol, 2.5 eq.) and Pd(dppf)Cl₃ (83.1 mg, 114 μmol, 0.1 eq.). The mixture was stirred under N₂ at 85° C. for 8 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue. The residue was purified by flash silica gel chromatography (from PE/EtOAc=1/0 to 5/1, TLC: PE/EtOAc=3/1, R_{f}=0.49) to yield 1-(4-(7-(benzyloxy)-3-phenyl-1H-isochromen-4-yl)-2-fluorophenyl)-4-(dimethoxymethyl)piperidine (500 mg, 884 μmol, 77.8%, 100% Purity) as a yellow solid. LC-MS (ESI⁺) m/z: 511.1 (M+H)⁺.

¹H NMR (400 MHz, CDCl₃) δ=7.45-7.31 (m, 5H), 7.24 (dd, J=3.0, 6.7 Hz, 2H), 7.18-7.12 (m, 3H), 6.95-6.85 (m, 3H), 6.78 (s, 3H), 5.21 (s, 2H), 5.08 (s, 2H), 4.11 (br d, J=3.2 Hz, 1H), 3.53 (d, J=11.7 Hz, 2H), 3.39 (s, 6H), 2.66 (t, J=11.4 Hz, 2H), 1.88 (d, J=13.4 Hz, 2H), 1.82-1.71 (m, 1H), 1.62-1.55 (m, 2H)

Step 3: To a solution of 1-(4-(7-(benzyloxy)-3-phenyl-1H-isochromen-4-yl)-2-fluorophenyl)-4-(dimethoxymethyl)piperidine (500 mg, 884 μmol, 1 eq.) in THF (10 mL) and MeOH (10 mL) was added Pd/C (500 mg, 10% Wt, 470 μmol, 0.532 eq.). The mixture was stirred under H₂ 20 psi at 60° C. for 24 h. The reaction mixture was filtered and concentrated to yield a residue. The residue were separated by chiral SFC (DAICEL CHIRALPAK AD (250 mm*30 mm, 10 um); mobile phase: CO₂-EtOH (0.1% NH₃H₂O); B %: 35%-35%, min), followed by lyophilization to yield (3S,4R)-4-(4-(4-(dimethoxymethyl)piperidin-1-yl)-3-fluorophenyl)-3-phenylisochroman-7-ol (180 mg, 0.36 mmol, 43%, 95% Purity, SFC: Rt=1.545) as a white solid and (3R,4S)-4-(4-(4-(dimethoxymethyl)piperidin-1-yl)-3-fluorophenyl)-3-phenylisochroman-7-ol (180 mg, 0.36 mmol, 43%, 95% Purity, SFC: R; =1.994) as a white solid. LC-MS (ESI⁺) m/z: 478.2 (M+H)⁺. ¹H NMR of 5 (500 MHz, CDCl₃) δ=7.19-7.11 (m, 3H), 7.00-6.95 (m, 2H), 6.91 (d, J=8.2 Hz, 1H), 6.68-6.56 (m, 3H), 6.46 (dd, J=1.8, 14.0 Hz, 1H), 6.40-6.35 (m, 1H), 5.19-5.10 (m, 1H), 5.06-4.98 (m, 2H), 4.07 (d, J=7.5 Hz, 1H), 3.98 (d, J=2.6 Hz, 1H), 3.36 (s, 8H), 2.56-2.45 (m, 2H), 1.79 (d, J=12.5 Hz, 2H), 1.69 (dt, J=3.8, 7.7 Hz, 1H), 1.48 (dq, J=3.8, 12.3 Hz, 2H).

¹H NMR of 5A (500 MHz, CDCl₃) δ=7.19-7.10 (m, 3H), 7.00-6.96 (m, 2H), 6.91 (d, J=8.2 Hz, 1H), 6.67-6.57 (m, 3H), 6.46 (dd, J=1.8, 13.9 Hz, 1H), 6.37 (dd, J=1.8, 8.2 Hz, 1H), 5.18-5.11 (m, 1H), 5.06-5.00 (m, 2H), 4.08 (d, J=7.5 Hz, 1H), 3.98 (d, J=2.9 Hz, 1H), 3.40-3.30 (m, 8H), 2.56-2.44 (m, 2H), 1.79 (d, J=12.2 Hz, 2H), 1.69 (tdt, J=3.8, 7.6, 11.5 Hz, 1H), 1.54-1.43 (m, 2H).

Step 4: To a solution of (35, 4R)-4-(4-(4-(dimethoxymethyl)piperidin-1-yl)-3-fluorophenyl)-3-phenylisochroman-7-ol (80 mg, 0.17 mmol, 1 eq.) in DCM (3 mL) was added 2,2,2-trifluoroacetic acid (19 mg, 0.8 mL, 0.17 mmol, 1 eq.). The mixture was stirred at 25° C. for 1 h. The reaction mixture was quenched by add to saturation solution of NaHCO₃ (20 mL), extracted with EtOAc (10 mL×3). The combined organic layers were washed with brine (10 mL), dried over Na₂SO₄, filtered and concentrated under reduced pressure to yield 1-(2-fluoro-4-((3S,4R)-7-hydroxy-3-phenylisochroman-4-yl)phenyl)piperidine-4-carbaldehyde (70 mg, 0.15 mmol, 87%, 90% Purity) as a yellow solid which was used in the next step without further purification. LC-MS (ESI⁺) m/z: 432.0 (M+H)⁺

Step 5: To a solution of (3R,4S)-4-(4-(4-(dimethoxymethyl)piperidin-1-yl)-3-fluorophenyl)-3-phenylisochroman-7-ol (80 mg, 0.17 mmol, 1 eq.) in DCM (3 mL) was added 2,2,2-trifluoroacetic acid (19 mg, 0.8 mL, 0.17 mmol, 1 eq.). The mixture was stirred at 25° C. for 1 h. The reaction mixture was quenched by add to saturation solution of NaHCO₃ (20 mL), extracted with EtOAc (10 mL×3). The combined organic layers were washed with brine (10 mL), dried over Na₂SO₄, filtered and concentrated under reduced pressure to yield 1-(2-fluoro-4-((3R,4S)-7-hydroxy-3-phenylisochroman-4-yl)phenyl)piperidine-4-carbaldehyde (70 mg, 0.15 mmol, 87%, 90% Purity) as a yellow solid which was used in the next step without further purification. LC-MS (ESI⁺) m/z: 432.0 (M+H)⁺.

Step 6: To a solution of 1-(2-fluoro-4-((3S,4R)-7-hydroxy-3-phenylisochroman-4-yl)phenyl) piperidine-4-carbaldehyde (70 mg, 90% Wt, 0.15 mmol, 1 eq.) and (S)-3-(1-oxo-5-(piperazin-1-yl)isoindolin-2-yl)piperidine-2,6-dione (72 mg, 0.22 mmol, 1.5 eq.) in DCM (3 mL) and MeOH (3 mL), after stirred 30 min was added Sodium triacetoxyborohydride (0.15 g, 0.11 mL, 0.73 mmol, 5 eq.). The mixture was stirred at 25° C. for 1 h. The reaction mixture was filtered and concentrated to yield a residue. The residue was purified by preparative HPLC (column: Phenomenex Gemini NX 150×30 mm, 5 μm; mobile phase: [water(NH₄HCO₃)-ACN]; B %: 46%-76%, 11 min), followed by lyophilization to yield (S)-3-(5-(4-((1-(2-fluoro-4-((3S,4R)-7-hydroxy-3-phenylisochroman-4-yl)phenyl)piperidin-4-yl)methyl)piperazin-1-yl)-1-oxoisoindolin-2-yl)piperidine-2,6-dione (72.0 mg, 96.2 μmol, 66%, 99.442% Purity) as a white solid. LC-MS (ESI⁺) m/z: 744.3 (M+H)⁺.

LCMS: calc. for C₄₄H₄₆FN₅O₅: 743.35, found: [M+H]⁺744.3.

HPLC: 99.442% purity at 220 nm.

¹H NMR (400 MHz, DMSO-d₆) δ=10.94 (s, 1H), 9.35 (s, 1H), 7.51 (d, J=9.0 Hz, 1H), 7.19-7.02 (m, 7H), 6.81 (d, J=8.1 Hz, 1H), 6.69-6.61 (m, 1H), 6.60-6.54 (m, 2H), 6.52-6.45 (m, 2H), 5.13-5.00 (m, 3H), 4.91 (d, J=15.3 Hz, 1H), 4.36-4.27 (m, 1H), 4.24-4.16 (m, 1H), 4.11 (d, J=2.6 Hz, 1H), 3.33-3.22 (m, 8H), 3.17 (d, J=10.5 Hz, 2H), 2.96-2.84 (m, 1H), 2.58 (d, J=16.5 Hz, 1H), 2.46-2.30 (m, 3H), 2.19 (d, J=4.9 Hz, 2H), 2.01-1.90 (m, 1H), 1.74 (d, J=11.5 Hz, 2H), 1.61 (s, 1H), 1.25-1.14 (m, 2H).

Step 7: To a solution of 1-(2-fluoro-4-((3R,4S)-7-hydroxy-3-phenylisochroman-4-yl)phenyl) piperidine-4-carbaldehyde (70 mg, 90% Wt, 0.15 mmol, 1 eq.) and (S)-3-(1-oxo-5-(piperazin-1-yl)isoindolin-2-yl)piperidine-2,6-dione (72 mg, 0.22 mmol, 1.5 eq.) in DCM (3 mL) and MeOH (3 mL), after stirred 30 min was added sodium triacetoxyhydroborate (0.15 g, 0.73 mmol, 5 eq.). The mixture was stirred at 25° C. for 1 h. The reaction mixture was filtered and concentrated to yield a residue. The residue was purified by preparative HPLC (column: Phenomenex Gemini NX 150×30 mm, 5 um; mobile phase: [water(NH₄HCO₃)-ACN]; B %: 46%-76%,11 min), followed by lyophilization to yield (S)-3-(5-(4-((1-(2-fluoro-4-((3R,4S)-7-hydroxy-3-phenylisochroman-4-yl)phenyl)piperidin-4-yl)methyl)piperazin-1-yl)-1-oxoisoindolin-2-yl)piperidine-2,6-dione (70.2 mg, 94.4 μmol, 65%, 100% Purity) as a white solid. LC-MS (ESI⁺) m/z: 744.3 (M+H)⁺.

LCMS: calc. for C₄₄H₄₆FN₅O₅: 743.35, found: [M+H]⁺744.3.

HPLC: 100% purity at 220 nm.

¹H NMR (400 MHz, DMSO-d₆) δ=10.94 (s, 1H), 9.34 (s, 1H), 7.51 (d, J=8.8 Hz, 1H), 7.18-7.03 (m, 7H), 6.81 (d, J=8.1 Hz, 1H), 6.68-6.62 (m, 1H), 6.60-6.54 (m, 2H), 6.52-6.46 (m, 2H), 5.12-5.00 (m, 3H), 4.91 (d, J=15.4 Hz, 1H), 4.36-4.28 (m, 1H), 4.24-4.16 (m, 1H), 4.11 (d, J=2.6 Hz, 1H), 3.33-3.21 (m, 8H), 3.17 (d, J=10.8 Hz, 2H), 2.96-2.84 (m, 1H), 2.58 (d, J=17.0 Hz, 1H), 2.46-2.27 (m, 3H), 2.19 (d, J=6.9 Hz, 2H), 2.00-1.92 (m, 1H), 1.74 (d, J=11.8 Hz, 2H), 1.61 (s, 1H), 1.25-1.14 (m, 2H).

### Example 132. Preparation of (I-385) (S)-3-(5-(4-((1-(5-((3S,4S)-7-hydroxy-3-phenylisochroman-4-yl)pyridin-2-yl)piperidin-4-yl)methyl)piperazin-1-yl)-1-oxoisoindolin-2-yl)piperidine-2,6-dione and (I-386) (S)-3-(5-(4-((1-(5-((3R,4R)-7-hydroxy-3-phenylisochroman-4-yl)pyridin-2-yl)piperidin-4-yl)methyl)piperazin-1-yl)-1-oxoisoindolin-2-yl)piperidine-2,6-dione

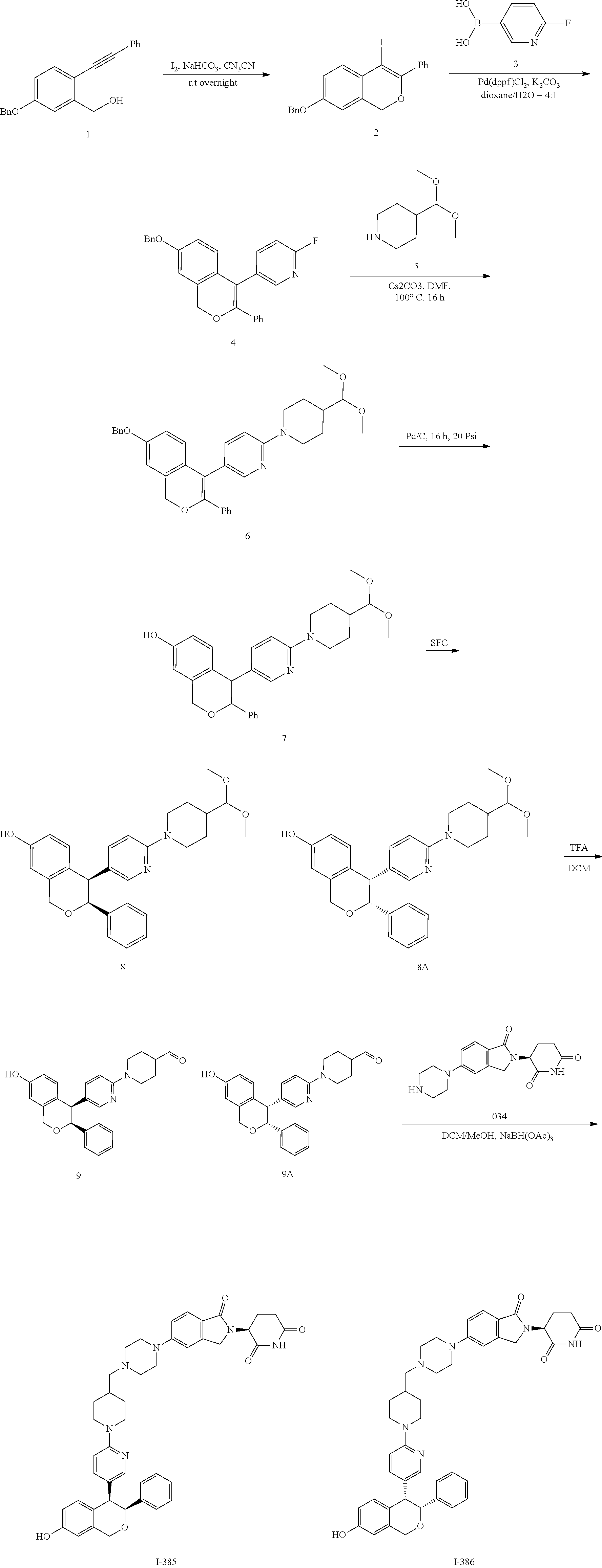

Step 1: To a solution of Compound 1 (2.00 g, 6.36 mmol, 1 eq.) in MeCN (100 mL) was added Sodium bicarbonate (1.60 g, 742 μL, 19.1 mmol, 3 eq.) and I₂ (4.84 g, 19.1 mmol, 3 eq.). The mixture was stirred at 20° C. for 12 hour. LCMS showed the reaction was completed. The reaction mixture was quenched by the addition of saturated aqueous Na₂S₂O₃ solution (100 mL) at 20° C. The mixture was extracted with EtOAc (100 mL×3). The combined organic layers were washed with brine (50 mL), dried over Na₂SO₄, filtered and concentrated. The crude material was purified on silica gel column chromatography (from pure PE to PE/EtOAc=15/1) to give Compound 2 (0.8 g, 2 mmol, 30%) as a black solid.

LC-MS (ESI⁺) m/z: 440.9 (M+H)⁺.

¹H NMR (400 MHz, DMSO-d₆) δ: ppm 7.63-7.73 (m, 2H), 7.43-7.54 (m, 7H), 7.37-7.42 (m, 1H), 7.31-7.36 (m, 1H), 7.08 (m, 1H), 6.96 (d, J=2.40 Hz, 1H), 5.24-5.30 (m, 2H), 5.20 (s, 2H).

Step 2: A mixture of Compound 2 (200 mg, 454 μmol, 1 eq.), Compound 3 (76.8 mg, 545 μmol, 1.2 eq.), PdCl₂(dppf) (33.2 mg, 45.4 μmol, 0.1 eq.) and K₂CO₃ (188 mg, 1.36 mmo, 3 eq.1) in Dioxane (12 mL) and Water (3 mL) was degassed and purged with N₂ for 3 times, and then the mixture was stirred at 80° C. for 3 hour under N₂ atmosphere. LCMS showed the reaction was completed. The reaction mixture was quenched by addition EA 10 mL at 20° C., and then diluted with water 30 mL and extracted with EA 30 mL (10 mL*3). The combined organic layers were washed with EA 30 mL (10 mL*3), dried over Na₂SO₄, filtered and concentrated under reduced pressure to give a residue. The crude material was purified on silica gel column chromatography (from pure PE to PE/EtOAc=5/1) to give Compound 4 (140 mg, 342 μmol, 75.3%) as a yellow solid.

LC-MS (ESI⁺) m/z: 410.2 (M+H)⁺.

Step 3: To a solution of Compound 4 (140.0 mg, 341.9 μmol, 1 eq.) and Compound 5 (65.33 mg, 410.3 μmol, 1.2 eq.) in DMF (10 mL) was added Cs₂CO₃ (334.2 mg, 1.026 mmol, 3 eq.). The mixture was stirred at 16° C. for 16 hour. LCMS showed the reaction was completed. The reaction mixture was quenched by addition EA 10 mL at 20° C., and then diluted with water 30 mL and extracted with EA 30 mL (10 mL*3). The combined organic layers were washed with EA 30 mL (10 mL*3), dried over Na₂SO₄, filtered and concentrated under reduced pressure to give a residue. The crude material was purified on silica gel column chromatography (from pure PE to PE/EtOAc=3/1) to give compound 6 (90 mg, 0.16 mmol, 48%) as a yellow solid.

LC-MS (ESI⁺) m/z: 549.8 (M+H)⁺.

Step 4: To a solution of Compound 6 (100 mg, 182 μmol, 1 eq.) in MeOH (2 mL) and THF (2 mL) was added Pd/C (10%, 0.03 g) under N₂ atmosphere. The suspension was degassed and purged with H₂ for 3 times. The mixture was stirred under H₂ 20 psi at 60° C. for 16 hours. LCMS showed the reaction was completed. The mixture was filtered and the filtrate was concentrated to give Compound 7 (50 mg, 0.11 mmol, 60%).

LC-MS (ESI⁺) m/z: 461.2 (M+H)⁺.

Step 5: The Compound 7 (50 mg, 0.11 mmol, 1 eq.) was purified by SFC (Column: DAICEL CHIRALCEL OD-H (250 mm*30 mm, 10 um); Mobile phase: 0.1% NH₃H₂O CO₂-ETOH; from 40% to 40%; Flow rate: 80 mL/min) to give Compound 8 (25 mg, 54 μmol, 50%) and Compound 8A (20 mg, 43 mmol, 40%) both as a white solid.

Compound 8: LC-MS (ESI⁺) m/z: 461.2 (M+H)⁺.

Compound 8A: LC-MS (ESI⁺) m/z: 461.2 (M+H)⁺.

Step 6: To a solution of Compound 8 (25 mg, 54 μmol, 1 eq.) in DCM (3 mL) and TFA (1 mL). The mixture was stirred at 20° C. for 1 hour. LCMS showed the reaction was completed. The mixture was concentrated in vacuo to give crude product. No further purification as it is used for the next step directly

LC-MS (ESI⁺) m/z: 415.2 (M+H)⁺

Step 7: To a solution of Compound 8A (25 mg, 54 μmol, 1 eq.) in DCM (3 mL) and TFA (1 mL). The mixture was stirred at 20° C. for 1 hour. LCMS showed the reaction was completed. The mixture was concentrated in vacuo to give crude product. No further purification as it is used for the next step directly.

LC-MS (ESI⁺) m/z: 415.2 (M+H)⁺.

Step 8: To a solution of Compound 9 (22 mg, 53 μmol, 1 eq.) and (S)-3-(1-oxo-5-(piperazin-1-yl)isoindolin-2-yl)piperidine-2,6-dione (21 mg, 64 μmol, 1.2 eq.) in DCM (3 mL) and MeOH (3 mL) was added sodium triacetoxyborohydride (34 mg, 0.16 mmol, 3 eq.). The mixture was stirred at 20° C. for 12 hour. LCMS showed the reaction was completed. The crude was purified by prep.HPLC together (Phenomenex Gemini NX 150×30 mm, 5 um, water(NH₄HCO₃)-ACN as a mobile phase, from 40% to 70%, Gradient Time (min): 11, Flow Rate (ml/min): 25) to give I-385 (15.8 mg, 21.7 μmol, 41%).

LCMS: calc. for C₄₃H₄₆N₆O₅:726.88, found: [M+H]⁺727.4 HPLC: 100.00% purity at 220 nm.

¹H NMR (500 MHz, METHANOL-d₄) δ: ppm 7.63-7.67 (m, 1H), 7.37-7.40 (m, 1H), 7.16-7.20 (m, 2H), 7.07-7.15 (m, 5H), 7.01 (m, 1H), 6.83-6.88 (m, 1H), 6.63-6.66 (m, 1H), 6.62 (d, J=2.40 Hz, 1H), 6.51-6.56 (m, 1H), 5.00-5.14 (m, 4H), 4.41 (d, J=8.40 Hz, 2H), 4.03-4.08 (m, 3H), 3.36-3.39 (m, 4H), 2.87-2.95 (m, 1H), 2.71-2.81 (m, 3H), 2.59-2.63 (m, 4H), 2.43-2.52 (m, 1H), 2.28 (d, J=6.80 Hz, 2H), 2.12-2.19 (m, 1H), 1.83 (d, J=13.20 Hz, 3H), 1.19 (s, 2H).

Step 9: To a solution of Compound 9A (15 mg, 36 μmol, 1 eq.) and (S)-3-(1-oxo-5-(piperazin-1-yl)isoindolin-2-yl)piperidine-2,6-dione (18 mg, 54 μmol, 1.5 eq.) in DCM (3 mL) and MeOH (3 mL) was added sodium triacetoxyborohydride (23 mg, 0.11 mmol, 3 eq.). The mixture was stirred at 20° C. for 12 hour. LCMS showed the reaction was completed. The crude was purified by prep.HPLC together (Phenomenex Gemini NX 150×30 mm, 5 um, water(NH₄HCO₃)-ACN as a mobile phase, from 40% to 70%, Gradient Time (min): 11, Flow Rate (ml/min): 25) to give I-386 (12.7 mg, 17.5 μmol, 48%).

LCMS: calc. for C₄₃H₄₆N₆O₅:726.88, found: [M+H]⁺727.4.

HPLC: 100.00% purity at 220 nm.

¹H NMR (500 MHz, METHANOL-d₄) δ: ppm 7.61-7.65 (m, 1H), 7.34-7.39 (m, 1H), 7.14-7.18 (m, 2H), 7.05-7.13 (m, 5H), 6.96-7.01 (m, 1H), 6.82-6.87 (m, 1H), 6.58-6.64 (m, 2H), 6.49-6.54 (m, 1H), 4.98-5.12 (m, 4H), 4.35-4.44 (m, 2H), 4.00-4.06 (m, 3H), 3.35 (d, J=9.60 Hz, 4H), 2.86-2.93 (m, 1H), 2.69-2.79 (m, 3H), 2.57-2.62 (m, 4H), 2.41-2.50 (m, 1H), 2.26 (d, J=6.40 Hz, 2H), 2.15 (m, 1H), 1.81 (d, J=12.80 Hz, 3H), 1.17 (s, 2H).

### Example 133. Preparation of (I-408) (S)-3-(1-oxo-5-(4-((1-(4-((6R,7S)-7-phenyl-6,7,8,9-tetrahydro-3H-benzo[e]indazol-6-yl)phenyl)piperidin-4-yl)methyl)piperazin-1-yl)isoindolin-2-yl)piperidine-2,6-dione and (I-44) (S)-3-(1-oxo-5-(4-((1-(4-((6S,7R)-7-phenyl-6,7,8,9-tetrahydro-3H-benzo[e]indazol-6-yl)phenyl)piperidin-4-yl)methyl)piperazin-1-yl)isoindolin-2-yl)piperidine-2,6-dione

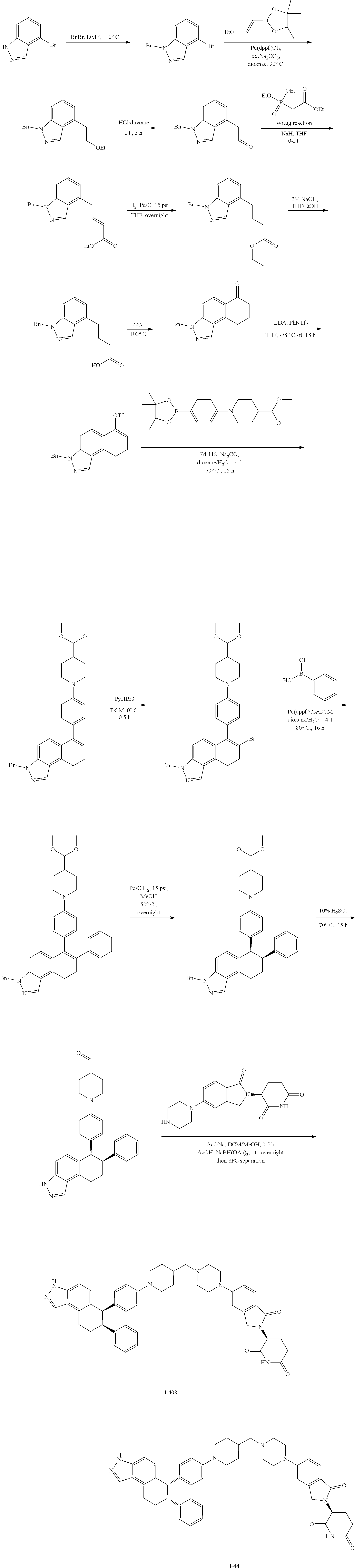

Step 1: To a solution of 4-bromo-1H-indazole (22 g, 1 Eq, 0.11 mol) in DMF (250 mL) was added Benzyl bromide (22 g, 15 mL, 1.15 Eq, 0.13 mol). The mixture was stirred at 110° C. for 16 hour. TLC (petroleum ether:ethyl acetate=10:1, R_{f}=0.3) indicated a new spot formed. The reaction was clean according to TLC. The solvent was removed under vacuum. The residue was dissolved in water (200 mL) and washed by ethyl acetate (2*100 mL). The combined organic layer was dried over Na₂SO₄, filtered and concentrated under vacuum to give the compound as a yellow oil. The reaction was purified by flash column (petroleum ether/ethyl acetate from 100/0 to 80/20) and the organic layer was concentrated in vacuo to give 1-benzyl-4-bromo-1H-indazole (20 g, 70 mmol, 62%) as a yellow solid.

Step 2: To a mixture of 1-benzyl-4-bromo-1H-indazole (20 g, 1 Eq, 70 mmol), (trans)-1-Ethoxyethene-2-boronic acid pinacol ester (15 g, 16 mL, 1.1 Eq, 77 mmol) and sodium carbonate (22 g, 13 mL, 3 Eq, 0.21 mol) in 1,4-dioxane (200 mL) and H₂O (50.0 mL) was added Pd(dppf) Cl₂ (5.7 g, 0.1 Eq, 7.0 mmol). The mixture was stirred overnight at 90° C. for 16 hour under N₂. TLC (petroleum ether:ethyl acetate=5:1, R_{f}=0.3) indicated a new spot formed. The reaction was clean according to TLC. The solvent was removed under vacuum. The residue was dissolved in water (200 mL) and washed by ethyl acetate (2*100 mL). The combined organic layer was dried over Na₂SO₄, filtered and concentrated under vacuum to give the compound as a yellow oil. The reaction was purified by flash column (petroleum ether/ethyl acetate from 100/0 to 70/30) and the organic layer was concentrated in vacuo to give (E)-1-benzyl-4-(2-ethoxyvinyl)-1H-indazole (14 g, 28 mmol, 40%, 55% Purity) as a yellow oil.

Step 3: To a solution of (E)-1-benzyl-4-(2-ethoxyvinyl)-1H-indazole (14 g, 55% Wt, 1 Eq, 28 mmol) in HCl/dioxane (40 mL). The mixture was stirred at 25° C. for 16 hr. TLC (PE/EtOAc=5/1, R_{f}=0.3) showed a new spot was detected. The solvent was removed under vacuo. The residue was dissolved in water (30 mL) and washed by ethyl acetate (2*15 mL). The combined organic layer was dried over Na₂SO₄, filtered and concentrated under vacuo to give the compound as a yellow oil. The reaction was purified by flash column (petroleum ether/ethyl acetate from 100/0 to 80/20) and the organic layer was concentrated in vacuo to give 2-(1-benzyl-1H-indazol-4-yl) acetaldehyde (6.7 g, 27 mmol, 97%) as a yellow oil.

Step 4: To a mixture of Sodium hydride (1.3 g, 60% Wt, 1.2 Eq, 32 mmol), in THE (100 mL) was added ethyl 2-(diethoxyphosphoryl)acetate (9.0 g, 8.0 mL, 1.5 Eq, 40 mmol), the resultant mixture was stirred for 30 min at 0° C. under N₂. 2-(1-benzyl-1H-indazol-4-yl) acetaldehyde (6.7 g, 1 Eq, 27 mmol) was added to the reaction. Then the resultant mixture was stirred for 2 hour at 0° C. under N₂. TLC (petroleum ether/ethyl acetate=3/1, R_{f}=0.3) showed a new point. The mixture was warmed to room temperature and treated with the saturation NH₄Cl aqueous solution (20 mL). The mixture was extracted with EA (30 mL*3), washed with H₂O (30 mL) and brine (30 mL). The combined organic layers were dried over anhydrous Na₂SO₄, filtered and concentrated under reduced pressure to give the residue which was purified by flash column (petroleum ether/ethyl acetate from 100/0 to 50/50) and the organic layer was concentrated in vacuo to give ethyl(E)-4-(1-benzyl-1H-indazol-4-yl) but-2-enoate (7 g, 0.02 mol, 60%, 70% Purity) as a yellow oil. LC-MS (ESI⁺) m/z: 321.2 (M+H)⁺.

Step 5: To a solution of ethyl(E)-4-(1-benzylindazol-4-yl) but-2-enoate (6.11 g, 17.17 mmol, 90% purity, 1 eq) in THF (50 mL) was added Pd/C (1.10 g, 1.03 mmol, 10% purity, 6.02e-2 eq) under N₂ atmosphere. The suspension was degassed and purged with H₂ for 2 times. The mixture was stirred under H₂ (15 Psi) at 25° C. for 16 hr. The reaction was filtered and the filter cake was pour into H₂O, and recycle to catalyst storage. The filtrate was concentrated. Compound ethyl 4-(1-benzylindazol-4-yl) butanoate (5.4 g, 13.40 mmol, 78.05% yield, 80% purity) was obtained as a yellow oil. LC-MS (ESI⁺) m/z: 323.2 (M+H)⁺.

Step 6: To a solution of ethyl 4-(1-benzylindazol-4-yl) butanoate (5.4 g, 13.40 mmol, 80% purity, 1 eq) in THF (20 mL) and EtOH (20 mL) was added NaOH (2 M, 20 mL, 2.99 eq). The mixture was stirred at 20° C. for 12 hr. The reaction mixture was diluted with H₂O 50 mL and extracted with EtOAc 100 mL (50 mL*2). The combined organic layers were washed with saturated salt solution 30 mL, dried over Na₂SO₄, filtered and concentrated under reduced pressure to give a yellow oil. Compound 4-(1-benzylindazol-4-yl) butanoic acid (3.9 g, 13.25 mmol, 98.88% yield) was obtained as a yellow oil. LC-MS (ESI⁺) m/z: 295.2 (M+H)⁺.

Step 7: To a solution of 4-(1-benzylindazol-4-yl) butanoic acid (3.9 g, 13.25 mmol, 1 eq) in PPA (30 mL). The mixture was stirred at 100° C. for 1 hr. TLC (petroleum ether:ethyl acetate=3:1) indicated Reactant 1 was consumed completely and many new spots formed. The reaction was messy according to TLC. The reaction mixture was quenched by addition NaHCO₃ 300 mL at 0° C., and then diluted with H₂O 200 mL and extracted with EtOAc 200 mL (100 mL*2). The combined organic layers were washed with saturated salt solution 50 mL, dried over Na₂SO₄, filtered and concentrated under reduced pressure to give a yellow oil. The residue was purified by flash silica gel chromatography (ISCO®; 80 g SepaFlash® Silica Flash Column, Eluent of 0˜30% Ethyl acetate/Petroleum ether gradient @ 45 mL/min). Compound 3-benzyl-8,9-dihydro-7H-benzo[e]indazol-6-one (3.5 g, 11.40 mmol, 86.03% yield, 90% purity) was obtained as a yellow solid. LC-MS (ESI⁺) m/z: 277.1 (M+H)⁺

Step 8: To a solution of 3-benzyl-3,7,8,9-tetrahydro-6H-benzo[e]indazol-6-one (800 mg, 1 Eq, 2.89 mmol) in THF (30 mL) cooled at −78° C. under N₂, and LDA (465 mg, 2.17 mL, 2 molar, 1.5 Eq, 4.34 mmol) was added dropwise and stirred for 30 min. 1,1,1-trifluoro-N-phenyl-N-((trifluoromethyl) sulfonyl) methanesulfonamide (1.55 g, 1.5 Eq, 4.34 mmol) was added. The reaction mixture was stirred for 16 hours. TLC (petroleum ether/ethyl acetate=5/1, R_{f}=0.4) showed one new spot was formed. The reaction mixture was cooled to room temperature and diluted with water (200 mL). The solids were filtered out and to the filtrate was added saturated NH₄Cl (200 mL). The resulting mixture was extracted with ethyl acetate (200 mL×2) and the organic layers combined. The organic was washed with brine (200 mL×2), dried over anhydrous sodium sulfate and concentrated under vacuum. The residue was purified by flash silica gel chromatography (ISCO®; 4 g SepaFlash® Silica Flash Column, Eluent of 0˜50% Ethyl acetate/Petroleum ether gradient @ 20 mL/min). The organic layer was concentrated in vacuo to give 3-benzyl-8,9-dihydro-3H-benzo[e]indazol-6-yltrifluoromethanesulfonate (500 mg, 1.22 mmol, 42.3%) as a yellow oil.

Step 9: A mixture of Pd-118 (90.9 mg, 0.1 Eq, 142 μmol), 4-(dimethoxymethyl)-1-(4-(4,4,5,5-tetramethyl-1,3,2-dioxaborolan-2-yl)phenyl)piperidine (821 mg, 1.2 Eq, 1.70 mmol), 3-benzyl-8,9-dihydro-3H-benzo[e]indazol-6-yl trifluoromethanesulfonate( ) and Na₂CO₃ (452 mg, 3 Eq, 4.26 mmol) in dioxane (16 mL) and H₂O (4.0 mL) at r.t. N₂ was bubbled into the mixture for 5 min. The reaction mixture was heated at 70° C. for 12 hour. Then the reaction was cooled to room temperature. TLC (PE/EtOAc=2/1, R=0.4) showed one new spot was formed. The mixture was treated with H₂O (50 mL), extracted with EtOAc (50 mL*2). The combined extracts were dried over anhydrous Na₂SO₄, filtered and concentrated to dryness in vacuo to give a residue. The residue was subjected to column chromatography over silica gel (gradient elution: 0˜20% ethyl acetate in petroleum ether). The pure fractions were collected and concentrated to dryness in vacuo to give 3-benzyl-6-(4-(4-(dimethoxymethyl)piperidin-1-yl)phenyl)-8,9-dihydro-3H-benzo[e]indazole (400 mg, 810 μmol, 57.1%) as a white solid.

Step 10: To a solution of 3-benzyl-6-[4-[4-(dimethoxymethyl)-1-piperidyl]phenyl]-8,9-dihydrobenzo[e]indazole (400 mg, 810.31 μmol, 1 eq) in DCM (3 mL) was added BLAH;pyridin-1-ium (272.11 mg, 850.83 μmol, 1.05 eq) and N-ethyl-N-isopropyl-propan-2-amine (157.09 mg, 1.22 mmol, 211.71 μL, 1.5 eq). The mixture was stirred at 0° C. for 0.5 hr. The reaction mixture was concentrated under reduced pressure to remove DCM. The residue was purified by column chromatography (SiO₂, Petroleum ether/Ethyl acetate=1/0 to 3/1). Compound 3-benzyl-7-bromo-6-[4-[4-(dimethoxymethyl)-1-piperidyl]phenyl]-8,9-dihydrobenzo[e]indazole (260 mg, 317.89 μmol, 39.23% yield, 70% purity) was obtained as a yellow oil.

LC-MS (ESI⁺) m/z: 572.1 (M+H)⁺

Step 11: A mixture of 3-benzyl-7-bromo-6-[4-[4-(dimethoxymethyl)-1-piperidyl]phenyl]-8,9-dihydrobenzo[e]indazole (220 mg, 384.26 μmol, 1 eq), phenylboronic acid (56.22 mg, 461.11 μmol, 1.2 eq), cyclopentyl(diphenyl)phosphane;dichloromethane;dichloropalladium;iron (31.38 mg, 38.43 μmol, 0.1 eq), na2co3 (122.18 mg, 1.15 mmol, 3 eq) in dioxane (2 mL) and H₂O (0.2 mL) was degassed and purged with N₂ for 3 times, and then the mixture was stirred at 100° C. for 16 hr under N₂ atmosphere. The reaction mixture was quenched by addition 5 mL H₂O, and then diluted with 20 mL EA and extracted with EA 40 mL (20 mL*2). The combined organic layers were washed with saturated salt water 10 mL, dried over anhydrous sodium sulfate, filtered and concentrated under reduced pressure to give a residue. The residue was purified by column chromatography (SiO₂, Petroleum ether/Ethyl acetate=0/1 to 3/1). The crude product 3-benzyl-6-[4-[4-(dimethoxymethyl)-1-piperidyl]phenyl]-7-phenyl-8,9-dihydrobenzo[e]indazole (196 mg, 344.02 μmol, 89.53% yield) as yellow solid was used into the next step without further purification.

LC-MS (ESI⁺) m/z: 570.2 (M+H)⁺

Step 12: To a solution of 3-benzyl-6-[4-[4-(dimethoxymethyl)-1-piperidyl]phenyl]-7-phenyl-8,9-dihydrobenzo[e]indazole (190 mg, 333.49 μmol, 1 eq) in MeOH (10 mL) was added Pd/C (380.00 mg, 3.57 mmol, 10.71 eq) under N₂ atmosphere. The suspension was degassed and purged with H₂ (13.44 mg, 6.67 mmol, 20 eq) for 3 times. The mixture was stirred under H₂ (15 Psi) at 50° C. for 12 hr. The reaction mixture was filtered and the filter was concentrated. The crude product 6-[4-[4-(dimethoxymethyl)-1-piperidyl]phenyl]-7-phenyl-6,7,8,9-tetrahydro-3H-benzo[e]indazole (100 mg, 207.63 μmol, 62.26% yield) as white solid was used into the next step without further purification.

LC-MS (ESI⁺) m/z: 482.1 (M+H)⁺

Step 13: 6-[4-[4-(dimethoxymethyl)-1-piperidyl]phenyl]-7-phenyl-6,7,8,9-tetrahydro-3H-benzo[e]indazole (100 mg, 207.63 μmol, 1 eq) was added to THF (2 mL) and H₂SO₄ (2 mL, 10% purity). The mixture was stirred at 70° C. for 16 hr. The reaction mixture was concentrated under reduced pressure to remove THE and diluted with EA 5 mL. Then the mixture was acidified with Saturated sodium carbonate to PH=5 and extracted with EA 50 mL (25 mL*2). The combined organic layers were washed with saturated salt water 30 mL, dried over anhydrous sodium sulfate, filtered and concentrated under reduced pressure to give a yellow solid. The crude product 1-[4-(7-phenyl-6,7,8,9-tetrahydro-3H-benzo[e]indazol-6-yl)phenyl]piperidine-4-carbaldehyde (85 mg, 195.15 μmol, 93.99% yield) as yellow solid was used into the next step without further purification.

LC-MS (ESI⁺) m/z: 436.0. (M+H)⁺

Step 14: To a solution of 1-[4-(7-phenyl-6,7,8,9-tetrahydro-3H-benzo[e]indazol-6-yl)phenyl]piperidine-4-carbaldehyde (85.00 mg, 195.15 μmol, 1 eq) in DCM (1 mL) and MEOH (1 mL) was added acetic acid (23.44 mg, 390.30 μmol, 22.32 μL, 2 eq) (3S)-3-(1-oxo-5-piperazin-1-yl-isoindolin-2-yl)piperidine-2,6-dione (128.16 mg, 390.30 μmol, 2 eq) and sodium acetate (80.05 mg, 975.76 μmol, 5 eq). The mixture was stirred at 20° C. for 1 hr. Then the NaBH(OAc)₃ (82.72 mg, 390.30 μmol, 2 eq) added to the mixture and stirred at 20° C. for 12 hr. The reaction mixture was concentrated under reduced pressure to remove DCM and MeOH. The residue was purified by prep-HPLC (column: Boston Green ODS 150*30 mm*5 um; mobile phase: [water (FA)-ACN]; B %: 26%-56%, 7 min) to give desired compound (30 mg, yield 20.55%, purity 100%) as a white solid, which was further separated by SFC (column: DAICEL CHIRALCEL OJ (250 mm*30 mm, 10 um); mobile phase: [0.1% NH₃H₂O ETOH]; B %: 55%-55%, min).

The crude product (3S)-3-[1-oxo-5-[4-[[1-[4-[(6R,7S)-7-phenyl-6,7,8,9-tetrahydro-3H-benzo[e]indazol-6-yl]phenyl]-4-piperidyl]methyl]piperazin-1-yl]isoindolin-2-yl]piperidine-2,6-dione (13 mg, 17.38 μmol, 8.91% yield, N/A purity) was used into the next step without further purification.

The crude product (3S)-3-[1-oxo-5-[4-[[1-[4-[(6S, 7R)-7-phenyl-6,7,8,9-tetrahydro-3H-benzo[e]indazol-6-yl]phenyl]-4-piperidyl]methyl]piperazin-1-yl]isoindolin-2-yl]piperidine-2,6-dione (14 mg, 18.72 μmol, 9.59% yield, N/A purity) was used into the next step without further purification.

068:

LC-MS (ESI⁺) m/z: 749.3 (M+H)⁺

HPLC: 87% purity at 220 nm.

¹H NMR (400 MHZ, DMSO-d₆) δ=8.14 (s, 1H), 7.96 (s, 1H), 7.74 (br d, J=8.5 Hz, 1H), 7.18 (br d, J=6.3 Hz, 4H), 6.99 (br d, J=8.8 Hz, 2H), 6.87 (br s, 2H), 6.59 (br d, J=8.3 Hz, 2H), 6.33 (br d, J=8.8 Hz, 2H), 5.23-5.19 (m, 1H), 4.40 (s, 1H), 4.34 (br d, J=4.3 Hz, 1H), 4.28 (s, 1H), 3.57 (br d, J=11.3 Hz, 2H), 3.47 (br d, J=6.0 Hz, 2H), 3.33 (br s, 5H), 2.59 (br s, 6H), 2.31 (br d, J=12.5 Hz, 4H), 1.85 (br s, 3H), 1.26 (s, 6H)

068A:

LC-MS (ESI⁺) m/z: 749.4 (M+H)⁺

HPLC: 92.924% purity at 220 nm.

¹H NMR (400 MHZ, DMSO-d₆) δ=8.14 (br s, 1H), 8.02 (br s, 1H), 7.74 (br d, J=8.0 Hz, 1H), 7.23-7.15 (m, 4H), 7.00 (br s, 2H), 6.88-6.85 (m, 2H), 6.60 (br d, J=9.3 Hz, 2H), 6.33 (br d, J=7.8 Hz, 2H), 5.21 (br dd, J=4.5, 13.1 Hz, 1H), 4.45-4.38 (m, 1H), 4.34 (br s, 1H), 4.30-4.23 (m, 1H), 3.57 (br d, J=10.3 Hz, 2H), 3.48 (br s, 2H), 3.33 (br s, 6H), 2.58 (br s, 6H), 2.33-2.21 (m, 4H), 1.92-1.80 (m, 3H), 1.34-1.24 (m, 6H).

### Example 134. Preparation of (I-1) (5S,6R)—S-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalene-2-carboxylic acid

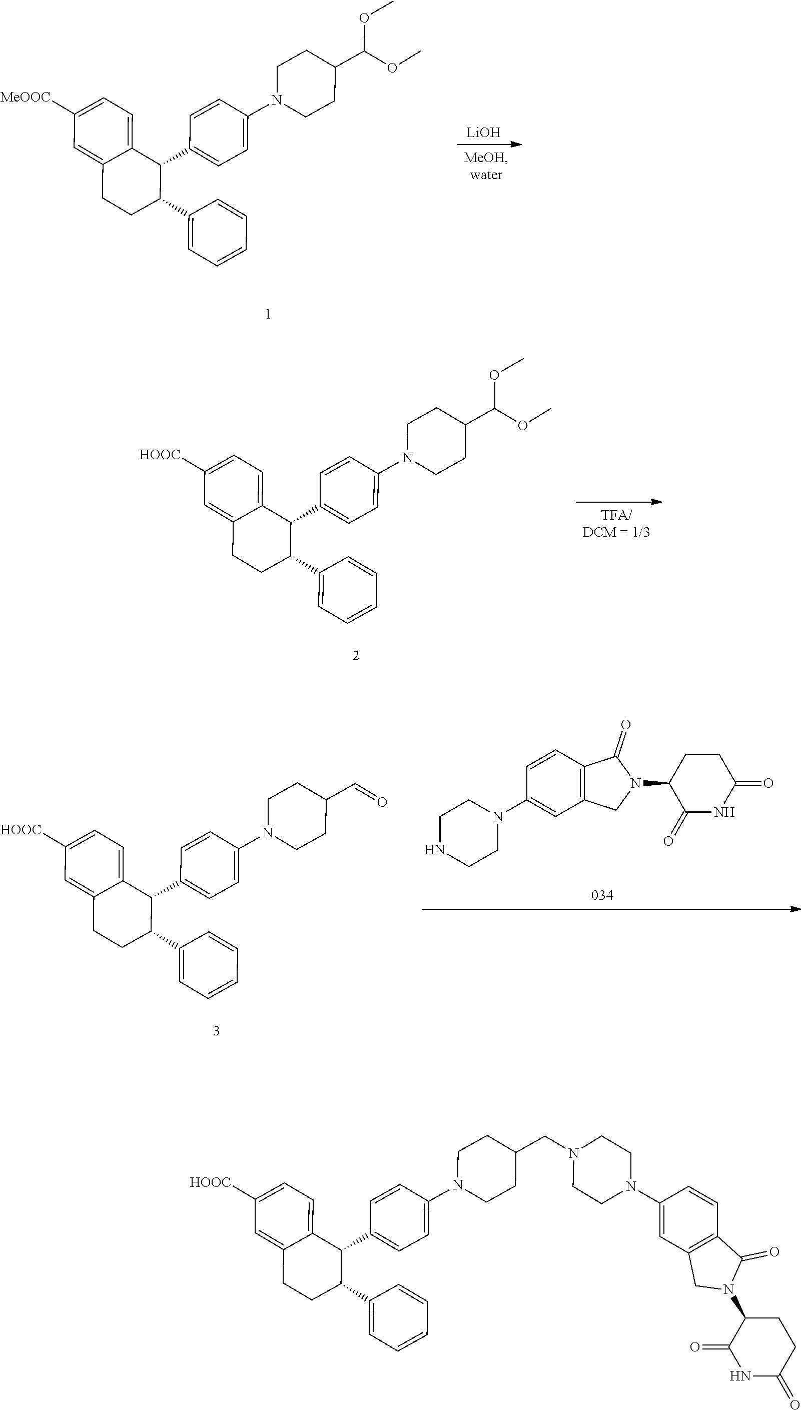

Step 1: To a mixture of compound 1 (110 mg, 220.16 μmol, 1 eq.) in MeOH (2 mL) and water (2 mL) was added LiOH (52.73 mg, 2.20 mmol, 10 eq.). The mixture was stirred at 25° C. for 16 hours. LCMS showed the reaction was completed. The mixture was diluted with saturated HCl aq. till pH=7. The mixture was concentrated in vacuo to give the residue which was re-crystallized from water, dried by lyophilization. Compound 2 (100 mg, 85.74% yield) was obtained as a yellow solid.

LC-MS (ESI⁺) m/z: 486.3 (M+H)⁺.

Step 2: To a mixture of compound 2 (100 mg, 205.93 μmol, 1 eq.) in TFA (1 mL) and DCM (3 mL) was stirred at 25° C. for 30 min. TLC (PE/EtOAc=3/1) showed the reaction was completed. The mixture was filtered and concentrated in vacuum. Compound 4 (90 mg, 99.43% yield) was obtained as a white solid.

Step 3: To a mixture of compound 3 (90 mg, 204.76 μmol, 1 eq.) and 034 (102.49 mg, 204.76 μmol, 1 eq., TsOH) in DCE (8 mL) was added sodium;triacetoxyboranuide (65.09 mg, 307.14 μmol, 1.5 eq.) and 4-methylmorpholine (20.71 mg, 204.76 μmol, 1 eq.) in one portion at 25° C. The mixture was stirred at 25° C. for 16 hours. LCMS (ES22193-4-R1A) showed the reaction was complete. The mixture was poured into water (5 mL). The aqueous phase was extracted with DCM (20 mL*3). The combined organic phase was dried with anhydrous Na₂SO₄, filtered and concentrated in vacuum. The residue was purified by prep-HPLC (Column: Welch Xtimate C18 150*25 mm*5 um, water (FA)-ACN as a mobile phase, from 22% to 52%, Gradient Time (min): 10, Flow Rate (ml/min): 25). 1-1 (27 mg, 17.54% yield, 100% purity) was obtained as a white solid.

LC-MS (ESI⁺) m/z: 752.6 (M+H)⁺.

¹H NMR (500 MHZ, METHANOL-d₄) δ ppm: 7.93 (s, 1H), 7.73-7.64 (m, 2H), 7.20-7.10 (m, 5H), 6.99 (d, J=8.0 Hz, 1H), 6.86-6.83 (m, 2H), 6.70-6.62 (m, 2H), 6.34 (d, J=8.5 Hz, 2H), 5.14-5.09 (m, 1H), 4.50-4.38 (m, 2H), 4.36 (d, J=5.0 Hz, 1H), 3.71 (s, 1H), 3.61 (d, J=8.5 Hz, 3H), 3.50-3.43 (m, 2H), 3.41 (s, 1H), 3.40-3.37 (m, 2H), 3.38 (d, J=4.0 Hz, 1H), 3.21-3.11 (m, 4H), 2.96-2.86 (m, 1H), 2.82-2.73 (m, 1H), 2.73-2.64 (m, 2H), 2.52-2.41 (m, 1H), 2.37-2.25 (m, 1H), 2.19-2.12 (m, 1H), 2.02 (d, J=8.5 Hz, 1H), 1.89 (d, J=9.5 Hz, 3H), 1.51-1.41 (m, 2H), 1.38-1.18 (m, 1H).

### Example 135. Preparation of (I-2) (5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalene-2-carboxylic acid

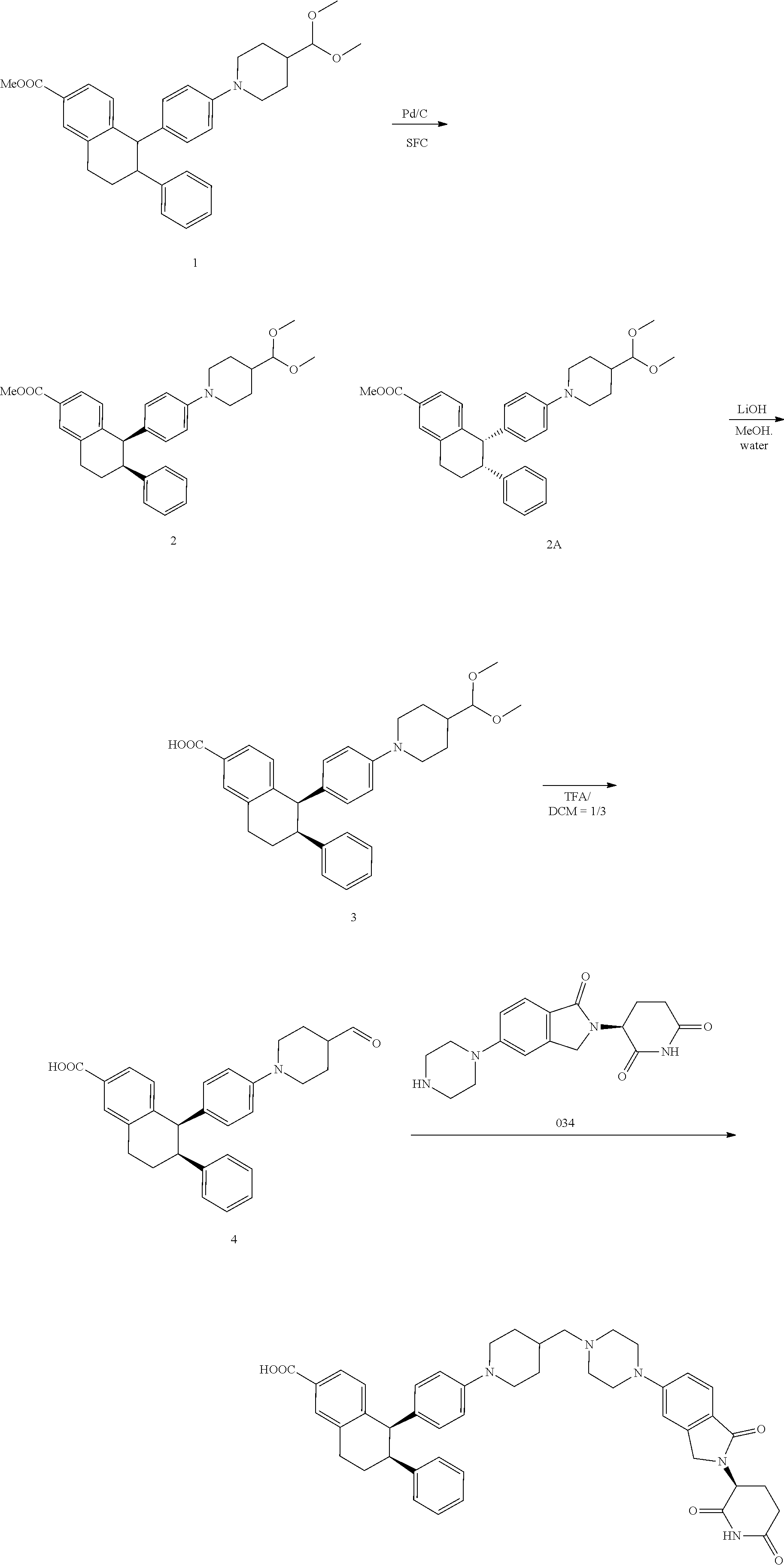

Step 1: To a mixture of compound 1 (400 mg, 803.82 μmol, 1 eq.) in MeOH (4 mL) and THF (2 mL) was added Pd/C (85.54 mg, 80.38 μmol, 10% purity, 0.1 eq.) in one portion at 25° C. under H₂. The mixture was stirred at 25° C. for 36 hours. LCMS (ES21801-14-R1A) showed the reaction was complete. The reaction mixture was filtered and concentrated under reduced pressure to give a residue. The residue was purified by SFC (condition: Column: DAICEL CHIRALCEL OJ (250 mm*30 mm, 10 um); Mobile phase: A: CO₂ B: ethanol (0.1% NH₃H₂O); Flow rate: 2.8 mL/min; Column temp.: 35° C.; ABPR: 1500 psi). Compound 2 (110 mg, 27.39% yield) and compound 2A (150 mg, 37.35% yield) were obtained as a yellow solid.

LC-MS (ESI⁺) m/z: 500.4 (M+H)⁺

Step 2: To a mixture of compound 2 (120 mg, 240.17 μmol, 1 eq.) in MeOH (2 mL) and water (2 mL) was added LiOH (57.52 mg, 2.40 mmol, 10 eq.). The mixture was stirred at 25° C. for 16 hours. LCMS showed the reaction was completed. The mixture was diluted with saturated HCl aq. till pH=7. The mixture was concentrated in vacuo to give the residue which was re-crystallized from water, dried by lyophilization. Compound 3 (100 mg, 85.74% yield) was obtained as a yellow solid.

LC-MS (ESI⁺) m/z: 486.3 (M+H)⁺.

Step 3: To a mixture of compound 3 (100 mg, 205.93 μmol, 1 eq.) in TFA (1 mL) and DCM (3 mL) was stirred at 25° C. for 30 min. TLC (PE/EtOAc=3/1) showed the reaction was completed. The mixture was filtered and concentrated in vacuum. Compound 4 (90 mg, 99.43% yield) was obtained as a white solid.

Step 4: To a mixture of compound 4 (90 mg, 204.76 μmol, 1 eq.) and 034 (102.49 mg, 204.76 μmol, 1 eq., TsOH) in DCE (8 mL) was added sodium;triacetoxyboranuide (65.09 mg, 307.14 μmol, 1.5 eq.) and 4-methylmorpholine (20.71 mg, 204.76 μmol, 1 eq.) in one portion at 25° C. The mixture was stirred at 25° C. for 16 hours. LCMS (ES22193-3-R1A) showed the reaction was complete. The mixture was poured into water (5 mL). The aqueous phase was extracted with DCM (20 mL*3). The combined organic phase was dried with anhydrous Na₂SO₄, filtered and concentrated in vacuum. The residue was purified by prep-HPLC (Column: Welch Xtimate C18 150*25 mm*5 um, water (FA)-ACN as a mobile phase, from 22% to 52%, Gradient Time (min): 10, Flow Rate (ml/min): 25). 1-2 (60 mg, 38.97% yield, 100% purity) was obtained as a white solid.

LC-MS (ESI⁺) m/z: 752.3 (M+H)⁺.

¹H NMR (500 MHZ, METHANOL-d₄) δ ppm: 7.93 (s, 1H), 7.70-7.65 (m, 2H), 7.15-7.11 (m, 5H), 6.99 (d, J=8.0 Hz, 1H), 6.86-6.82 (m, 2H), 6.65 (d, J=8.5 Hz, 2H), 6.32 (d, J=9.0 Hz, 2H), 5.13-5.08 (m, 1H), 4.45-4.36 (m, 2H), 4.35 (d, J=4.5 Hz, 1H), 3.58 (s, 2H), 3.49 (s, 3H), 3.42-3.36 (m, 2H), 3.33 (d, J=1.5 Hz, 1H), 3.24-3.19 (m, 1H), 3.15-3.09 (m, 1H), 2.98 (s, 4H), 2.92-2.85 (m, 1H), 2.81-2.74 (m, 1H), 2.67 (s, 1H), 2.62 (s, 2H), 2.51-2.41 (m, 1H), 2.37-2.29 (m, 1H), 2.18-2.11 (m, 1H), 1.87 (d, J=10.5 Hz, 4H), 1.42-1.35 (m, 2H).

### Example 136. Preparation of (I-7) (S)-5-(4-(4-((4-(2-(2,6-dioxopiperidin-3-yl)-1-oxoisoindolin-S-yl)piperazin-1-yl)methyl)piperidin-1-yl)phenyl)-6-phenyl-7,8-dihydronaphthalene-2-carboxylic acid

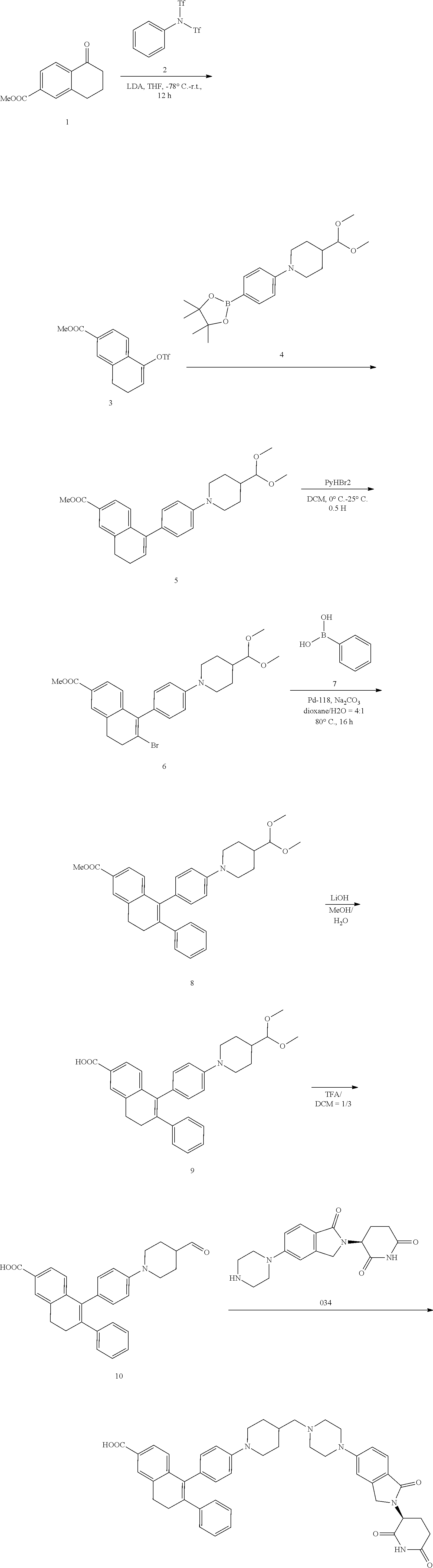

Step 1: To a mixture of compound 1 (3.5 g, 17.14 mmol, 1 eq.) in THF (100 mL) was added LDA (2 M, 12.85 mL, 1.5 eq.) in one portion at −75° C. under N₂. The mixture was stirred at −75° C. for 1 hour, then compound 2 (12.25 g, 34.28 mmol, 2 eq.) was added. The mixture was stirred at 25° C. for 12 hours. TLC (PE/EtOAc=3/1) showed the reaction was complete. The reaction mixture was quenched by water (20 mL) and extracted with ethyl acetate 150 mL (50 mL*3). The combined organic layers were washed with brine 150 mL (50 mL*3), dried over Na₂SO₄, filtered and concentrated under reduced pressure to give a residue. The residue was purified by column chromatography (SiO₂, Petroleum ether/Ethyl acetate=3/1 to 1/1) to give compound 3 (8 g, 15.70 mmol, 91.61% yield, 66% purity) as a yellow solid.

LC-MS (ESI⁺) m/z: 337.0 (M+H)⁺

¹H NMR (500 MHz, CHLOROFORM-d) δ ppm: 7.95-7.93 (m, 1H), 7.86 (d, J=1.0 Hz, 1H), 7.32-7.30 (m, 1H), 6.19-6.14 (m, 1H), 3.93 (s, 3H), 2.95-2.91 (m, 2H), 2.59-2.54 (m, 2H).

Step 2: To a mixture of compound 3 (500 mg, 1.49 mmol, 1 eq.) and compound 4 (537.17 mg, 1.49 mmol, 1 eq.) in dioxane (S mL) and Water (1 mL) was added pd (dppf) Cl₂ (108.79 mg, 148.68 μmol, 0.1 eq.) and K₂CO₃ (616.47 mg, 4.46 mmol, 3 eq.) in one portion at 25° C. under N₂. The mixture was stirred at 80° C. for 2 hours. LCMS (ES21801-1-PIA) indicated the reaction was complete. The mixture was added water (10 mL). The aqueous phase was extracted with ethyl acetate (50 mL*3). The combined organic phase was washed with brine (20 mL), dried with anhydrous Na₂SO₄, filtered and concentrated in vacuum. The residue was purified by silica gel chromatography (Petroleum ether/Ethyl acetate=1/1) to give compound 5 (400 mg, 948.93 μmol, 63.82% yield) as a white solid.

LC-MS (ESI⁺) m/z: 422.2 (M+H)⁺.

¹H NMR (400 MHZ, CHLOROFORM-d) δ ppm: 7.86 (d, J=1.2 Hz, 1H), 7.78-7.75 (m, 1H), 7.24-7.20 (m, 2H), 7.13 (d, J=8.0 Hz, 1H), 6.98-6.93 (m, 2H), 6.19-6.17 (m, 1H), 4.14-4.09 (m, 1H), 3.91 (s, 3H), 3.78-3.75 (m, 2H), 3.39 (s, 6H), 2.90-2.86 (m, 2H), 2.75-2.68 (m, 2H), 2.44-2.38 (m, 2H), 1.91-1.86 (m, 2H), 1.83-1.74 (m, 1H), 1.54-1.43 (m, 2H)

Step 3: To a mixture of compound 5 (400 mg, 948.93 μmol, 1 eq.) in DCM (4 mL) was added PyHBr₃ (212.44 mg, 664.25 μmol, 0.7 eq.) in one portion at 0° C. under N₂. The mixture was stirred at 25° C. for 30 min. LCMS (ES21801-5-P1B) showed the reaction was complete.

The reaction mixture was concentrated under reduced pressure to give a residue. The residue was purified by column chromatography (SiO₂, Petroleum ether/Ethyl acetate=3/1 to 1/1). Compound 6 (120 mg, 25.27% yield) was obtained as a white solid.

LC-MS (ESI⁺) m/z: 501.8 (M+H)⁺.

Step 4: To a mixture of compound 6 (120 mg, 239.80 μmol, 1 eq.) and compound 7 (29.24 mg, 239.80 μmol, 1 eq.) in dioxane (3 mL) and Water (0.1 mL) was added pd (dppf) Cl₂ (17.55 mg, 23.98 μmol, 0.1 eq.) and K₂CO₃ (99.42 mg, 719.39 μmol, 3 eq.) in one portion at 25° C. under N₂. The mixture was stirred at 80° C. for 16 hours. LCMS (ES21801-9-R1A) showed the reaction was complete. The reaction mixture was quenched by addition water 20 mL and extracted with ethyl acetate 150 mL (50 mL*3). The combined organic layers were washed with brine 150 mL (50 mL*3), dried over Na₂SO₄, filtered and concentrated under reduced pressure to give a residue. The residue was purified by column chromatography (SiO₂, Petroleum ether/Ethyl acetate=3/1 to 1/1). Compound 8 (100 mg, 83.80% yield) was obtained as a yellow solid.

LC-MS (ESI⁺) m/z: 498.3 (M+H)⁺.

Step 5: To a mixture of compound 8 (100 mg, 201 μmol, 1 eq.) in Water (3 mL) and MeOH (3 mL) was added LiOH (17.3 mg, 402 μmol, 2 eq.) in one portion. The mixture was stirred at 25° C. for 16 hours. LCMS showed the reaction was completed. The mixture was diluted with saturated HCl aq. till pH=7. The mixture was concentrated in vacuo to give the residue which was re-crystallized from water, dried by lyophilization. Compound 9 (60 mg, 62%) was obtained as a white solid.

LC-MS (ESI⁺) m/z: 484.2 (M+H)⁺.

Step 6: To a mixture of compound 9 (60 mg, 0.12 mmol, 1 eq.) in TFA (1 mL) and DCM (3 mL) was stirred at 25° C. for 30 min. TLC (PE/EtOAc=3/1) showed the reaction was completed. The mixture was filtered and concentrated in vacuum. Compound 10 (50 mg, 92%) was obtained as a white solid.

Step 7: To a mixture of compound 10 (50 mg, 114.28 μmol, 1 eq.) and 034 (57.20 mg, 114.28 μmol, 1 eq., TsOH) in DCE (5 mL) was added sodium;triacetoxyboranuide (48.44 mg, 228.56 μmol, 2 eq.) and 4-methylmorpholine (1.16 mg, 11.43 μmol, 0.1 eq.) in one portion at 25° C. The mixture was stirred at 25° C. for 16 hours. LCMS (ES21801-13-R1A) showed the reaction was complete. The mixture was poured into water (5 mL). The aqueous phase was extracted with DCM (20 mL*3). The combined organic phase was dried with anhydrous Na₂SO₄, filtered and concentrated in vacuum. The residue was purified by prep-HPLC (Column: Welch Xtimate C18 150*25 mm*5 um, water (FA)-ACN as a mobile phase, from 22% to 52%, Gradient Time (min): 10, Flow Rate (ml/min): 25)). I-7 (9.5 mg, 11.09% yield, 100% purity) was obtained as a white solid.

LC-MS (ESI⁺) m/z: 750.3 (M+H)⁺.

¹H NMR (500 MHZ, METHANOL-d₄) δ ppm: 7.85 (d, J=1.5 Hz, 1H), 7.70-7.64 (m, 2H), 7.13-7.04 (m, 7H), 6.90 (s, 4H), 6.82 (d, J=9.0 Hz, 1H), 5.13-5.08 (m, 1H), 4.46-4.36 (m, 2H), 3.72-3.66 (m, 2H), 3.44-3.42 (m, 4H), 3.03-2.99 (m, 2H), 2.95-2.85 (m, 1H), 2.82-2.70 (m, 9H), 2.49-2.40 (m, 3H), 2.18-2.12 (m, 1H), 1.94-1.90 (m, 2H), 1.86-1.77 (m, 1H), 1.44-1.34 (m, 2H).

### Example 137. Preparation of (I-135) (3S)-3-[5-[4-[[1-[4-[(1R,2R)-2-cycloheptyl-6-hydroxy-tetralin-1-yl]phenyl]-4-piperidyl]methyl]piperazin-1-yl]-1-oxo-isoindolin-2-yl]piperidine-2,6-dione and (I-134) (35)-3-[5-[4-[[1-[4-[(1S,2S)-2-cycloheptyl-6-hydroxy-tetralin-1-yl]phenyl]-4-piperidyl]methyl]piperazin-1-yl]-1-oxo-isoindolin-2-yl]piperidine-2,6-dione

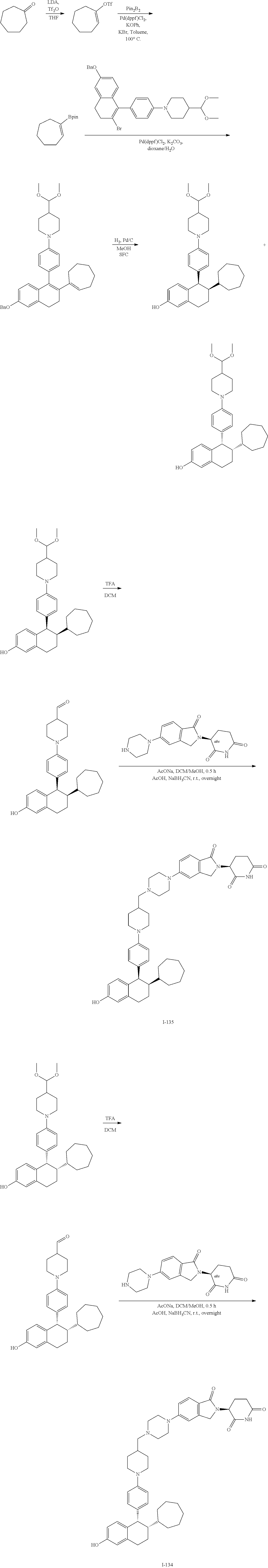

Step 1: To a solution of cycloheptanone (1 g, 8.92 mmol, 1.05 mL, 1 eq) and Na₂CO₃ (1.89 g, 17.83 mmol, 2 eq) in DCM (50 mL) was added Tf₂O (4.10 g, 14.55 mmol, 2.4 mL, 1.63 eq) under 0° C. The mixture was stirred at 25° C. for 12 h. The reaction mixture was quenched by addition of water (50 mL), extracted with DCM (30 mL×3). The combined organic layers were washed with brine (10 mL), dried over Na₂SO₄, filtered and concentrated under reduced pressure to give a residue. The residue was purified by flash silica gel chromatography (from pure PE to PE/EtOAc=5/1, TLC: PE/EtOAc=5/1, R_{f}=0.82) to yield cyclohepten-1-yl trifluoromethanesulfonate (1.8 g, 6.6 mmol, 74.4% yield, 90% purity) as yellow oil.

¹H NMR (500 MHz, CDCl₃) δ=5.89 (t, J=6.4 Hz, 1H), 2.57-2.49 (m, 2H), 2.20-2.11 (m, 2H), 1.76-1.68 (m, 4H), 1.64 (td, J=5.6, 10.9 Hz, 2H).

Step 2: To a solution of cyclohepten-1-yl trifluoromethanesulfonate (800 mg, 2.95 mmol, 1 eq) and Pin₂B₂ (973.20 mg, 3.83 mmol, 1.3 eq) in toluene (10 mL) was added Pd(dppf) Cl₂ (107.85 mg, 147.40 μmol, 0.05 eq) potassium phenolate (584.60 mg, 4.42 mmol, 1.5 eq) and KBr (526.23 mg, 4.42 mmol, 191.36 μL, 1.5 eq). The mixture was stirred at 50° C. for 2 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue. The residue was purified by flash silica gel chromatography (from PE/EtOAc=1/0 to 5/1, TLC: PE/EtOAc=5/1, R_{f}=0.67) to yield 2-(cyclohept-1-en-1-yl)-4,4,5,5-tetramethyl-1,3,2-dioxaborolane (470 mg, 2.01 mmol, 68.18% yield, 95% purity) as yellow oil.

¹H NMR (400 MHZ, CDCl₃) δ=6.78 (t, J=6.4 Hz, 1H), 2.25 (d, J=5.7, 18.8 Hz, 4H), 1.79-1.71 (m, 2H), 1.52-1.44 (m, 4H), 1.26 (s, 12H).

Step 3: To a solution of 1-(4-(6-(benzyloxy)-2-bromo-3,4-dihydronaphthalen-1-yl)phenyl)-4-(dimethoxymethyl)piperidine (900 mg, 1.64 mmol, 1 eq) and 2-(cyclohept-1-en-1-yl)-4,4,5,5-tetramethyl-1,3,2-dioxaborolane3 (422.02 mg, 1.80 mmol, 95% purity, 1.1 eq) in dioxane (9 mL) and H₂O (3 mL) was added Pd(dppf)Cl₃ (120.06 mg, 164.08 μmol, 0.1 eq) and Na₂CO₃ (434.77 mg, 4.10 mmol, 2.5 eq). The mixture was stirred at 100° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue. The residue was purified by flash silica gel chromatography (from PE/EtOAc=1/0 to 10/1, TLC: PE/EtOAc=5/1, R_{f}=0.68) to yield 1-(4-(6-(benzyloxy)-2-(cyclohept-1-en-1-yl)-3,4-dihydronaphthalen-1-yl)phenyl)-4-(dimethoxymethyl)piperidine (500 mg, 824.81 μmol, 50.27% yield, 93% purity) as a yellow solid. LC-MS (ESI⁺) m/z 564.4 [M+H]⁺.

¹H NMR (400 MHZ, CDCl₃) δ=7.45-7.29 (m, 5H), 7.03 (br d, J=8.2 Hz, 2H), 6.90 (br d, J=8.2 Hz, 2H), 6.80 (d, J=2.4 Hz, 1H), 6.71-6.65 (m, 1H), 6.64-6.59 (m, 1H), 5.50 (br t, J=6.4 Hz, 1H), 5.04 (s, 2H), 4.12 (t, J=7.1 Hz, 1H), 3.73 (br d, J=12.2 Hz, 2H), 3.39 (s, 6H), 2.82 (t, J=7.7 Hz, 2H), 2.74-2.62 (m, 2H), 2.44 (dd, J=6.7, 8.8 Hz, 2H), 2.05 (br d, J=10.4 Hz, 2H), 2.00-1.84 (m, 4H), 1.82-1.72 (m, 1H), 1.67-1.59 (m, 2H), 1.55-1.44 (m, 2H), 1.39-1.28 (m, 4H).

Step 4: To a solution of 1-(4-(6-(benzyloxy)-2-(cyclohept-1-en-1-yl)-3,4-dihydronaphthalen-1-yl)phenyl)-4-(dimethoxymethyl)piperidine (500 mg, 824.81 μmol, 93% purity, 1 eq) in MeOH (10 mL) was added Pd/C (500 mg, 10% purity, 1.00 eq). The mixture was stirred under H₂ (15 Psi) at 25° C. for 1 b. The reaction mixture was filtered concentrated to yield a residue. The residue was purified by Chiral SFC (column: DAICEL CHIRALPAK AD (250 mm*30 mm, 10 um); mobile phase: [0.1% NH₃H₂O ETOH]; B %: 40%-40%, min), followed by lyophilization to yield (1R,2R)-2-cycloheptyl-1-[4-[4-(dimethoxymethyl)-1-piperidyl]phenyl]tetralin-6-ol (150 mg, 314.02 μmol, 37.5% yield, 100% purity, SFC: Rt=2.481) as a white solid and (1S,2S)-2-cycloheptyl-1-[4-[4-(dimethoxymethyl)-1-piperidyl]phenyl]tetralin-6-ol (160 mg, 324.91 μmol, 38.8% yield, 97% purity, SFC: Rt=3.873) as a white solid. LC-MS (ESI⁺) m/z 478.4 [M+H]⁺.

¹H NMR of (1R,2R)-2-cycloheptyl-1-[4-[4-(dimethoxymethyl)-1-piperidyl]phenyl]tetralin-6-ol: (400 MHz, CDCl₃) δ=6.88 (d, J=7.4 Hz, 2H), 6.77 (br d, J=8.2 Hz, 3H), 6.61 (s, 1H), 6.53-6.45 (m, 1H), 4.76-4.49 (m, 1H), 4.15 (br d, J=4.3 Hz, 1H), 4.09 (br d, J=7.0 Hz, 1H), 3.64 (br d, J=12.3 Hz, 2H), 3.37 (s, 6H), 3.00-2.90 (m, 1H), 2.88-2.74 (m, 1H), 2.61 (br s, 2H), 1.90-1.68 (m, 6H), 1.67-1.60 (m, 2H), 1.53-1.32 (m, 10H), 1.29-1.14 (m, 2H), 1.04-0.91 (m, 1H).

¹H NMR of (1S,2S)-2-cycloheptyl-1-[4-[4-(dimethoxymethyl)-1-piperidyl]phenyl]tetralin-6-ol: (400 MHz, CDCl₃)>=6.91-6.85 (m, 2H), 6.83-6.74 (m, 3H), 6.61 (d, J=2.4 Hz, 1H), 6.52-6.45 (m, 1H), 4.75 (br s, 1H), 4.14 (d, J=4.5 Hz, 1H), 4.08 (d, J=7.4 Hz, 1H), 3.64 (br d, J=11.8 Hz, 2H), 3.37 (s, 6H), 2.99-2.90 (m, 1H), 2.86-2.75 (m, 1H), 2.61 (t, J=12.0 Hz, 2H), 1.86-1.71 (m, 6H), 1.67-1.63 (m, 2H), 1.50-1.34 (m, 10H), 1.28-1.20 (m, 2H), 1.04-0.94 (m, 1H).

Step 5: To a solution of (1R,2R)-2-cycloheptyl-1-[4-[4-(dimethoxymethyl)-1-piperidyl]phenyl]tetralin-6-ol (70.00 mg, 146.54 μmol, 100% purity, 1 eq) in DCM (2.5 mL) was added TFA (898.33 mg, 7.88 mmol, 583.33 μL, 53.76 eq). The mixture was stirred at 25° C. for 1 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield 1-[4-[(1R,2R)-2-cycloheptyl-6-hydroxy-tetralin-1-yl]phenyl]piperidine-4-carbaldehyde (50 mg, 115.85 μmol, 79.05% yield, N/A purity) as a white solid which was used in the next step without further purification. LC-MS (ESI⁺) m/z 432.4 [M+H]⁺.

Step 6: To a solution of 1-[4-[(1R,2R)-2-cycloheptyl-6-hydroxy-tetralin-1-yl]phenyl]piperidine-4-carbaldehyde (50.00 mg, 115.85 μmol, N/A purity, 1 eq) and (35)-3-(1-oxo-5-piperazin-1-yl-isoindolin-2-yl)piperidine-2,6-dione (38.04 mg, 115.85 μmol, 1 eq) in DCM (2 mL) and MeOH (2 mL) after stirred 30 min was added NaBH(OAc)₃ (98.21 mg, 463.38 μmol, 4 eq). The mixture was stirred at 25° C. for 12 h. The reaction mixture was concentrated to yield a residue. The residue was purified by preparative HPLC (column: Welch Xtimate C18 150*25 mm*5 um; mobile phase: [water(NH₄HCO₃)-ACN]; B %: 75%-95%, 11 min), followed by lyophilization to yield (35)-3-[5-[4-[[1-[4-[(1R,2R)-2-cycloheptyl-6-hydroxy-tetralin-1-yl]phenyl]-4-piperidyl]methyl]piperazin-1-yl]-1-oxo-isoindolin-2-yl]piperidine-2,6-dione (45.5 mg, 61.16 μmol, 52.79% yield, 100% purity) as a white solid. LC-MS (ESI⁺) m/z 417.2 [M+H]⁺.

LCMS: calc. for C₄₆H₅₇N₅O₄:743.44, found: [M+H]⁺744.5.

HPLC: 100% purity at 220 nm.

¹H NMR (500 MHz, CD₃OD) δ=7.63 (d, J=8.5 Hz, 1H), 7.10-7.05 (m, 2H), 6.93-6.84 (m, 4H), 6.65 (d, J=8.2 Hz, 1H), 6.55 (d, J=2.4 Hz, 1H), 6.45 (dd, J=2.6, 8.4 Hz, 1H), 5.10 (dd, J=5.1, 13.4 Hz, 1H), 4.45-4.35 (m, 2H), 4.09 (d, J=4.6 Hz, 1H), 3.60 (br d, J=12.1 Hz, 2H), 3.39-3.35 (m, 4H), 2.95-2.86 (m, 2H), 2.81-2.74 (m, 2H), 2.70-2.58 (m, 6H), 2.46 (dq, J=4.5, 13.2 Hz, 1H), 2.31 (d, J=7.2 Hz, 2H), 2.18-2.10 (m, 1H), 1.90 (br d, J=12.5 Hz, 2H), 1.84 (br t, J=7.5 Hz, 1H), 1.79-1.62 (m, 5H), 1.53-1.42 (m, 6H), 1.40-1.29 (m, 4H), 1.26-1.10 (m, 2H), 1.01-0.92 (m, 1H).

Step 7: To a solution of (1S,2S)-2-cycloheptyl-1-[4-[4-(dimethoxymethyl)-1-piperidyl]phenyl]tetralin-6-ol (70 mg, 142.15 μmol, 97% purity, 1 eq) in DCM (2.5 mL) was added TFA (871.36 mg, 7.64 mmol, 565.82 μL, 53.76 eq). The mixture was stirred at 25° C. for 1 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield 1-[4-[(1S,2S)-2-cycloheptyl-6-hydroxy-tetralin-1-yl]phenyl]piperidine-4-carbaldehyde (50 mg, 115.85 μmol, 81.5% yield, N/A purity) as a white solid which was used in the next step without further purification. LC-MS (ESI⁺) m/z 432.4 [M+H]⁺.

Step 8: To a solution of 1-[4-[(1S,2S)-2-cycloheptyl-6-hydroxy-tetralin-1-yl]phenyl]piperidine-4-carbaldehyde (50.00 mg, 115.85 μmol, N/A purity, 1 eq) and (35)-3-(1-oxo-5-piperazin-1-yl-isoindolin-2-yl)piperidine-2,6-dione (38.04 mg, 115.85 μmol, 1 eq) in DCM (2 mL) and MeOH (2 mL) after stirred 30 min was added NaBH(OAc)₃ (98.21 mg, 463.38 μmol, 4 eq). The mixture was stirred at 25° C. for 12.5 h. The reaction mixture was concentrated to yield a residue. The residue was purified by preparative HPLC (column: Welch Xtimate C18 150*25 mm*5 um; mobile phase: [water(NH₄HCO₃)-ACN]; B %: 75%-95%, 11 min), followed by lyophilization to yield (3S)-3-[5-[4-[[1-[4-[(1S,2S)-2-cycloheptyl-6-hydroxy-tetralin-1-yl]phenyl]-4-piperidyl]methyl]piperazin-1-yl]-1-oxo-isoindolin-2-yl]piperidine-2,6-dione (44.8 mg, 60.22 μmol, 51.98% yield, 100% purity) as a white solid. LC-MS (ESI⁺) m/z 744.6 [M+H]⁺.

LCMS: calc. for C₄₆H₅₇N₅O₄:743.44, found: [M+H]⁺744.6.

HPLC: 100% purity at 220 nm.

¹H NMR (500 MHz, CD₃OD) δ=7.63 (d, J=8.4 Hz, 1H), 7.11-7.05 (m, 2H), 6.93-6.85 (m, 4H), 6.65 (d, J=8.4 Hz, 1H), 6.55 (d, J=2.4 Hz, 1H), 6.45 (dd, J=2.6, 8.4 Hz, 1H), 5.10 (dd, J=5.1, 13.4 Hz, 1H), 4.45-4.34 (m, 2H), 4.09 (d, J=4.7 Hz, 1H), 3.60 (br d, J=11.9 Hz, 2H), 3.39-3.35 (m, 4H), 2.95-2.85 (m, 2H), 2.82-2.73 (m, 2H), 2.68-2.58 (m, 6H), 2.46 (dq, J=4.6, 13.2 Hz, 1H), 2.31 (d, J=7.0 Hz, 2H), 2.19-2.10 (m, 1H), 1.90 (br d, J=12.5 Hz, 2H), 1.84 (br t, J=7.9 Hz, 1H), 1.78-1.62 (m, 5H), 1.54-1.42 (m, 5H), 1.40-1.29 (m, 4H), 1.23-1.06 (m, 2H), 1.02-0.93 (m, 1H).

### Example 138. Preparation of (I-163) (S)-3-(5-(4-((1-(4-((1S,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

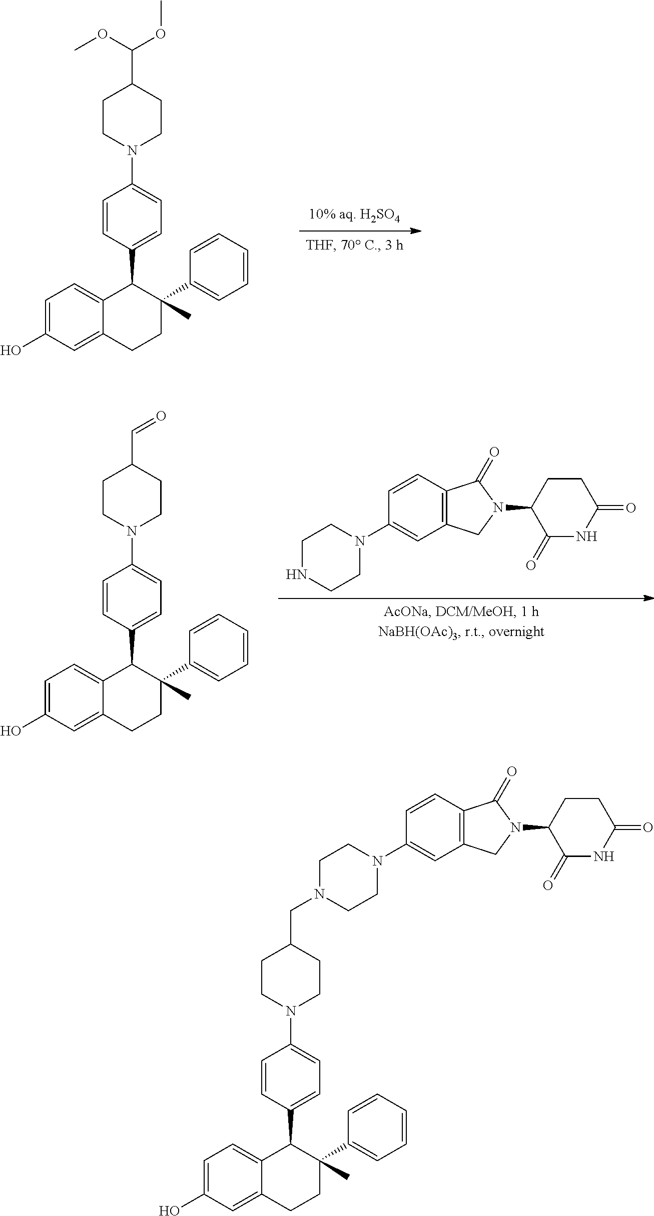 [SMILES: COC(OC)C1CCN(c2ccc([C@@H]3c4ccc(O)cc4CC[C@@]3(C)c3ccccc3)cc2)CC1 ; C[C@@]1(c2ccccc2)CCc2cc(O)ccc2[C@H]1c1ccc(N2CCC(C=O)CC2)cc1 ; O=C1CC[C@H](N2Cc3cc(N4CCNCC4)ccc3C2=O)C(=O)N1 ; C[C@@]1(c2ccccc2)CCc2cc(O)ccc2[C@H]1c1ccc(N2CCC(CN3CCN(c4ccc5c(c4)CN([C@H]4CCC(=O)NC4=O)C5=O)CC3)CC2)cc1]

Step 1: To a solution of (5S,6R)-5-(4-(4-(dimethoxymethyl)piperidin-1-yl)phenyl)-6-methyl-6-phenyl-5,6,7,8-tetrahydronaphthalen-2-ol (66 mg, 1 Eq, 0.14 mmol) in THF (8 mL) was added 10% sulfuric acid (0.14 g, 8 mL). The mixture was stirred at 70° C. for 2 hour. LCMS showed 98.828% desired MS. The mixture was adjusted to pH=8-9 by progressively adding NaHCO₃. The reaction mixture was quenched by addition 100 mL EtOAc at 20° C., and then diluted with 50 mL H₂O and extracted with EtOAc 50 mL (25 mL*2). The combined organic layers were washed with 10 mL (10 mL*1), dried over Na₂SO₄. The reaction was filtered and concentrated under reduced pressure to give 1-(4-((1S, 2R)-6-hydroxy-2-methyl-2-phenyl-1,2,3,4-tetrahydronaphthalen-1-yl)phenyl)piperidine-4-carbaldehyde (80 mg, 0.19 mmol, crude, 98.828% purity) was obtained as a brown oil. LC-MS (ESI⁺) m/z: 444.2 (M+H)⁺.

Step 2: A mixture of 1-(4-((1S,2R)-6-hydroxy-2-methyl-2-phenyl-1,2,3,4-tetrahydronaphthalen-1-yl)phenyl)piperidine-4-carbaldehyde (80 mg, 1 Eq, 0.19 mmol), (S)-3-(1-oxo-5-(piperazin-1-yl)isoindolin-2-yl)piperidine-2,6-dione, Tosylic acid (93 mg, 1 Eq, 0.19 mmol) and sodium acetate (76 mg, 5 Eq, 0.93 mmol) in DCM (2 mL) and MeOH (2 mL) at 20° C. for 0.5 hour, pH=8, then acetic acid (33 mg, 32 μL, 3 Eq. 0.56 mmol) at 20° C. for 1 hour, pH=6, then added sodium triacetoxyhydroborate (79 mg, 2 Eq, 0.37 mmol) the mixture, and then the mixture was stirred at 25° C. for 16 hour. LCMS showed 60.52% desired MS. The reaction was concentrated to give (S)-3-(5-(4-((1-(4-((1S,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione as a brown oil. The mixture was filtered and the filter cake was washed by EtOAc (50 mL×3). The brown oil was purified by preparative high-performance liquid chromatography.

Condition: Column: Welch Xtimate C18 150*30 mm*5 um; A: water (FA); B: ACN at the beginning: A (79%) and B (21%); at the end: A: (49%) and B (51%); Gradient Time (min) 7; 100% B hold Time (min) 3.2, Flow Rate (ml/min) 25.

The pure fractions were collected and the solvent was evaporated under vacuum. The aqueous layer was lyophilized to dryness to give (S)-3-(5-(4-((1-(4-((1S,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (80.5 mg, 108 μmol, 58% yield, 98.66% purity) as a white solid.

LCMS: calc. for C₄₆H₅₁N₅O₄: 737.39, found: [M+H]⁺738.5.

HPLC: 98.66% purity at 220 nm.

¹H NMR (400 MHZ, DMSO-d₆) δ=10.95 (s, 1H), 8.98 (s, 1H), 8.13 (s, 0.344H), 7.52 (d, J=8.7 Hz, 1H), 7.39 (d, J=7.7 Hz, 2H), 7.21 (t, J=7.7 Hz, 2H), 7.12-7.03 (m, 3H), 6.77 (s, 4H), 6.67 (d, J=8.3 Hz, 1H), 6.44-6.35 (m, 2H), 5.09-5.00 (m, 1H), 4.44 (s, 1H), 4.33 (d, J=17.0 Hz, 1H), 4.24-4.16 (m, 1H), 3.61 (br d, J=11.7 Hz, 2H), 3.38-3.34 (m, 6H), 2.95-2.84 (m, 1H), 2.79-2.71 (m, 1H), 2.59 (br d, J=2.4 Hz, 2H), 2.56 (br s, 2H), 2.45-2.36 (m, 2H), 2.36-2.21 (m, 3H), 2.12-2.04 (m, 1H), 2.00-1.92 (m, 1H), 1.91-1.84 (m, 1H), 1.80 (br d, J=11.7 Hz, 2H), 1.73-1.62 (m, 1H), 1.25-1.14 (m, 2H), 0.95 (s, 3H)

SFC: retention time, 2.434 min; Area, 91.36%;

### Example 139. Preparation of (I-344) (S)-3-(5-(4-((1-(4-((1R,2S)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

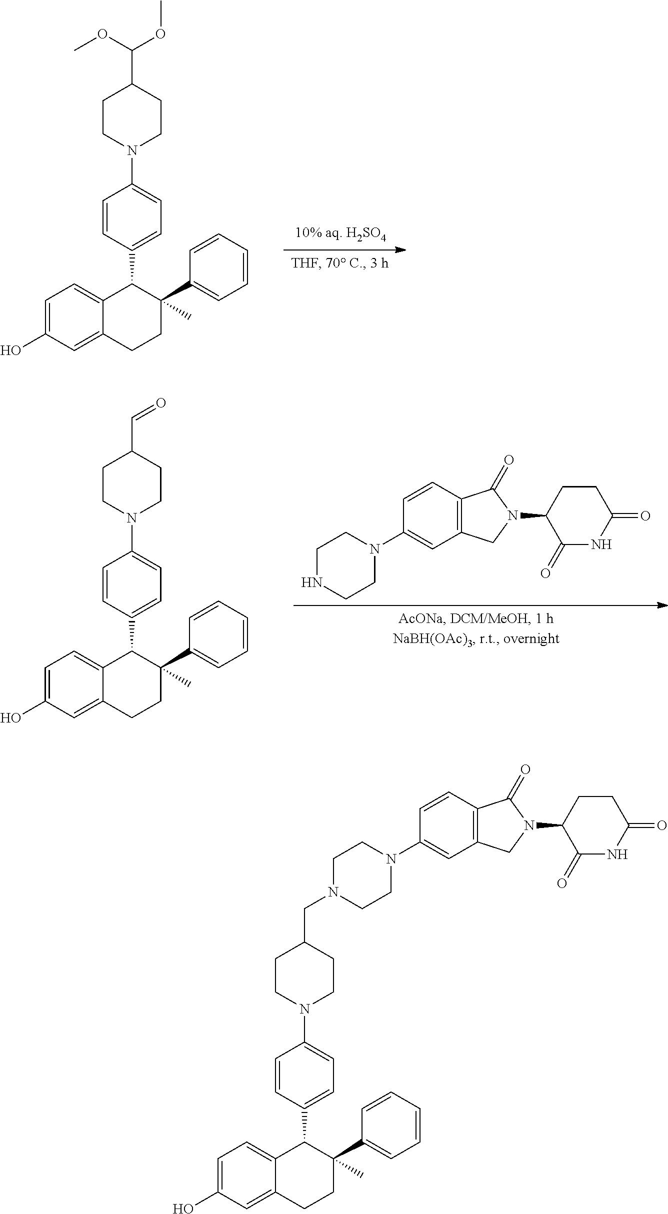 [SMILES: COC(OC)C1CCN(c2ccc([C@H]3c4ccc(O)cc4CC[C@]3(C)c3ccccc3)cc2)CC1 ; C[C@]1(c2ccccc2)CCc2cc(O)ccc2[C@@H]1c1ccc(N2CCC(C=O)CC2)cc1 ; O=C1CC[C@H](N2Cc3cc(N4CCNCC4)ccc3C2=O)C(=O)N1 ; C[C@]1(c2ccccc2)CCc2cc(O)ccc2[C@@H]1c1ccc(N2CCC(CN3CCN(c4ccc5c(c4)CN([C@H]4CCC(=O)NC4=O)C5=O)CC3)CC2)cc1]

Step 1: To a solution of (5R,6S)-5-(4-(4-(dimethoxymethyl)piperidin-1-yl)phenyl)-6-methyl-6-phenyl-5,6,7,8-tetrahydronaphthalen-2-ol (62 mg, 1 Eq, 0.13 mmol) in THF (8 mL) was added 10% sulfuric acid (0.13 g, 8 mL). The mixture was stirred at 70° C. for 2 hour. LCMS showed 98.86% desired MS. The mixture was adjusted to pH=8-9 by progressively adding NaHCO₃. The reaction mixture was quenched by addition 100 mL EtOAc at 20° C., and then diluted with 50 mL H₂O and extracted with EtOAc (25 mL*2). The combined organic layers were washed with 10 mL brine, dried over Na₂SO₄. The reaction was filtered and concentrated under reduced pressure to give 1-(4-((1R,2S)-6-hydroxy-2-methyl-2-phenyl-1,2,3,4-tetrahydronaphthalen-1-yl)phenyl)piperidine-4-carbaldehyde (75 mg, 0.17 mmol, crude, 98.86% purity) was obtained as a brown oil. LC-MS (ESI⁺) m/z: 444.2 (M+H)⁺.

Step 2: A mixture of 1-(4-((1R,2S)-6-hydroxy-2-methyl-2-phenyl-1,2,3,4-tetrahydronaphthalen-1-yl)phenyl)piperidine-4-carbaldehyde (75 mg, 1 Eq, 0.17 mmol), (S)-3-(1-oxo-5-(piperazin-1-yl)isoindolin-2-yl)piperidine-2,6-dione, PhSO₃H salt (87 mg, 1 Eq, 0.17 mmol) and sodium acetate (71 mg, 5 Eq, 0.87 mmol) in DCM (2 mL) and MeOH (2 mL) at 20° C. for 0.5 hour, pH=8, then acetic acid (31 mg, 30 μL, 3 Eq, 0.52 mmol) at 20° C. for 1 hour, pH=6, then added sodium triacetoxyhydroborate (74 mg, 2 Eq, 0.35 mmol) the mixture, and then the mixture was stirred at 25° C. for 16 hour. LCMS showed 62.46% desired MS. The reaction was concentrated to give (S)-3-(5-(4-((1-(4-((1S,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione as a brown oil. The mixture was filtered and the filter cake was washed by EtOAc (50 mL×3). The brown oil was purified by preparative high-performance liquid chromatography.

Condition: Column: Welch Xtimate C18 150*30 mm*5 um; A: water (FA); B: CAN; at the beginning: A (79%) and B (21%); at the end: A: (49%) and B (51%); Gradient Time (min) 7; 100% B hold Time (min) 3.2, Flow Rate (ml/min) 25.

The pure fractions were collected and the solvent was evaporated under vacuum. The aqueous layer was lyophilized to dryness to give (S)-3-(5-(4-((1-(4-((1R,2S)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (80.5 mg, 108 μmol, 62% yield, 99.23% purity) as a white solid.

LCMS: calc. for C₄₆H₅₁N₅O₄:737.39, found: [M+H]⁺738.5.

HPLC: 99.23% purity at 220 nm.

¹H NMR (400 MHZ, DMSO-d₆) δ=10.95 (s, 1H), 8.98 (s, 1H), 8.13 (s, 0.435H), 7.52 (d, J=8.6 Hz, 1H), 7.39 (d, J=7.7 Hz, 2H), 7.21 (t, J=7.7 Hz, 2H), 7.12-7.03 (m, 3H), 6.77 (s, 4H), 6.67 (d, J=8.3 Hz, 1H), 6.44-6.36 (m, 2H), 5.05 (dd, J=5.1, 13.3 Hz, 1H), 4.44 (s, 1H), 4.33 (d, J=17.0 Hz, 1H), 4.24-4.16 (m, 1H), 3.61 (br d, J=11.1 Hz, 2H), 3.33 (br s, 6H), 2.90 (br s, 1H), 2.73 (br s, 1H), 2.59 (br d, J=2.6 Hz, 2H), 2.55 (br s, 3H), 2.37 (br s, 1H), 2.36-2.30 (m, 1H), 2.24 (br d, J=6.2 Hz, 2H), 2.07 (br d, J=4.6 Hz, 1H), 1.98-1.92 (m, 1H), 1.91-1.84 (m, 1H), 1.80 (br d, J=10.8 Hz, 2H), 1.72-1.63 (m, 1H), 1.26-1.14 (m, 2H), 0.95 (s, 3H)

SFC: retention time, 2.246 min; Area, 91.17%;

### Example 140. Preparation of (I-157) (3S)-3-[5-[4-[[1-[4-[(1R,2R)-6-hydroxy-2-(2,2,2-trifluoroethyl)tetralin-1-yl]phenyl]-4-piperidyl]methyl]piperazin-1-yl]-1-oxo-isoindolin-2-yl]piperidine-2,6-dione

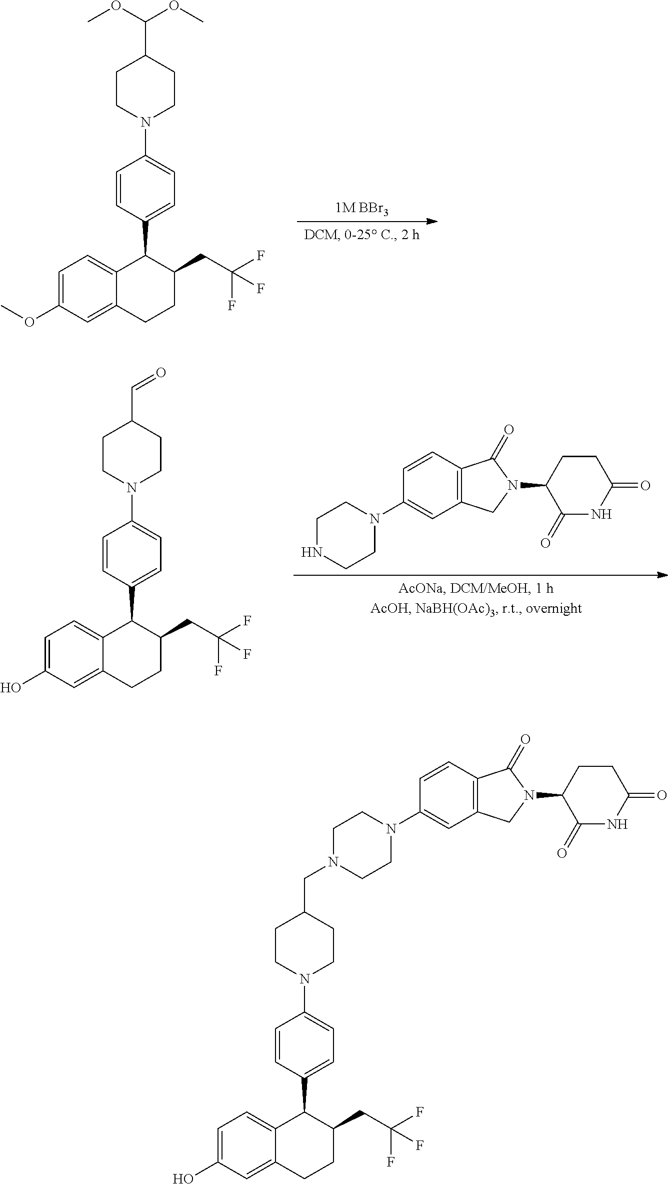

Step 1: A mixture of 4-(dimethoxymethyl)-1-(4-((1R,2R)-6-methoxy-2-(2,2,2-trifluoroethyl)-1,2,3,4-tetrahydronaphthalen-1-yl)phenyl)piperidine (65 mg, 136.11 μmol, 1 eq) in DCM (4 mL) was degassed and purged with N₂ for 3 times, and then BBr₃ (1 M, 408.33 μL, 3 eq) was degassed the mixture at 0° C. and was stirred at 25° C. for 16 hr under N₂ atmosphere, LCMS showed 95% of desired compound was detected. The reaction mixture was quenched with NaHCO₃20 mL, and then was treated with 20 mL H₂O and extracted with DCM 50 mL (50 mL*2). The organic layers were dried with anhydrous Na₂SO₄, filtered, The reaction mixture was filtered and concentrated to dryness in vacuo to give 1-[4-[(1S,2S)-6-hydroxy-2-(2,2,2-trifluoroethyl)tetralin-1-yl]phenyl]piperidine-4-carbaldehyde (50 mg, 119.77 μmol, 88.00% yield) was obtained as a white solid. LC-MS (ESI⁺) m/z: 416 (M+H)⁺.

Step 2: A mixture of (3S)-3-(1-oxo-5-piperazin-1-yl-isoindolin-2-yl)piperidine-2,6-dione (89.93 mg, 179.66 μmol, 1.5 eq, PhSO₃H salt), 1-[4-[(1R,2R)-6-hydroxy-2-(2,2,2-trifluoroethyl)tetralin-1-yl]phenyl]piperidine-4-carbaldehyde (50 mg, 119.77 μmol, 1 eq), sodium acetate (29.48 mg, 359.31 μmol, 3 eq) was added in DCM (4 mL) MeOH (4 mL), and the mixture was stirred at 30° C. for 1 h, and then NaBH(OAc)₃ (50.77 mg, 239.54 μmol, 2 eq) and acetic acid (99.21 mg, 359.31 μmol, 94.49 μL, 3 eq) was added in the mixture, and then the mixture was stirred at 30° C. for 12 hr. LCMS showed 72% of desired compound was detected. The reaction mixture was treated with H₂O 30 mL and extracted with DCM (50 mL*2). The organic layers were dried with anhydrous Na₂SO₄, filtered, the residue was purified by prep. HPLC (column: Welch Xtimate C18 150*30 mm*5 um; mobile phase: [water (FA)-ACN]; gradient: 18%-48% B over 7 min). The aqueous phase was lyophilized to dryness to give (38)-3-[5-[4-[[1-[4-[(1R,2R)-6-hydroxy-2-(2,2,2-trifluoroethyl)tetralin-1-yl]phenyl]-4-piperidyl]methyl]piperazin-1-yl]-1-oxo-isoindolin-2-yl]piperidine-2,6-dione (12.6 mg, 17.26 μmol, 14.41% yield) was obtained as a yellow solid. LC-MS (ESI⁺) m/z: 730.2 (M+H)⁺.

HPLC: 92.601%, purity at 220 nm.

¹H NMR (400 MHZ, DMSO-d₆) δ=10.99-10.92 (m, 1H), 9.17-9.04 (m, 1H), 8.17-8.10 (m, 0.313H), 7.58-7.48 (m, 1H), 7.12-7.02 (m, 2H), 6.88-6.76 (m, 4H), 6.65-6.60 (m, 1H), 6.56-6.50 (m, 1H), 6.48-6.41 (m, 1H), 5.10-5.00 (m, 1H), 4.40-4.28 (m, 1H), 4.25-4.17 (m, 1H), 4.03-3.94 (m, 1H), 3.69-3.58 (m, 2H), 3.32-3.23 (m, 5H), 2.99-2.78 (m, 3H), 2.70-2.55 (m, 5H), 2.43-2.17 (m, 6H), 2.02-1.91 (m, 1H), 1.86-1.54 (m, 7H), 1.28-1.10 (m, 2H).

SFC: retention time, 2.273 min; Area, 93.205%;

### Example 141. Preparation of (I-158) (3S)-3-[5-[4-[[1-[4-[(1S,2S)-6-hydroxy-2-(2,2,2-trifluoroethyl)tetralin-1-yl]phenyl]-4-piperidyl]methyl]piperazin-1-yl]-1-oxo-isoindolin-2-yl]piperidine-2,6-dione

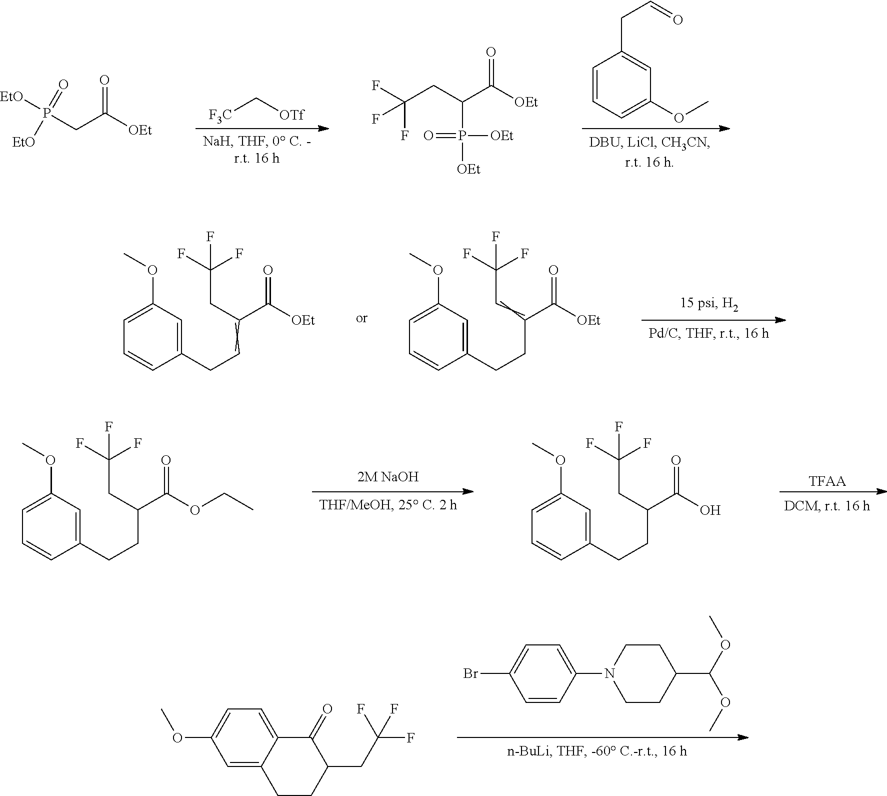

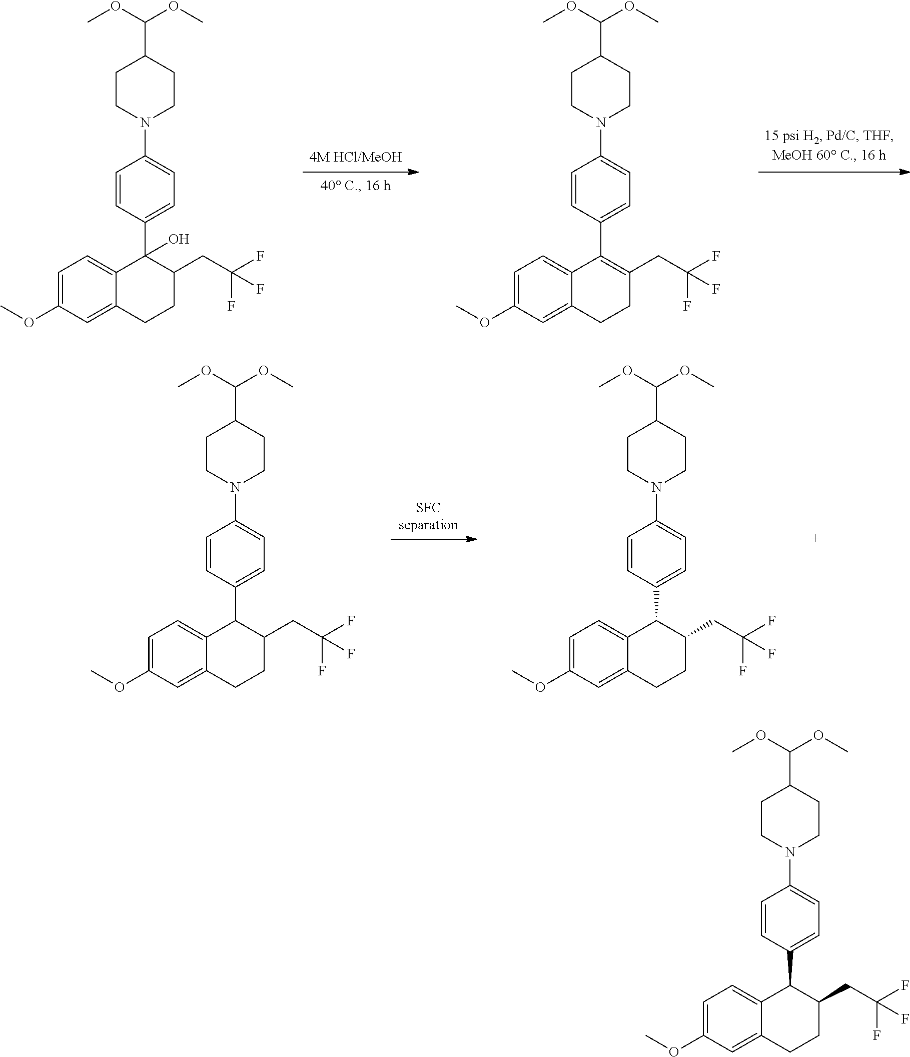 [SMILES: COc1ccc2c(c1)CCC(CC(F)(F)F)C2(O)c1ccc(N2CCC(C(OC)OC)CC2)cc1 ; COc1ccc2c(c1)CCC(CC(F)(F)F)=C2c1ccc(N2CCC(C(OC)OC)CC2)cc1 ; COc1ccc2c(c1)CCC(CC(F)(F)F)C2c1ccc(N2CCC(C(OC)OC)CC2)cc1 ; COc1ccc2c(c1)CC[C@@H](CC(F)(F)F)[C@H]2c1ccc(N2CCC(C(OC)OC)CC2)cc1 ; COc1ccc2c(c1)CC[C@H](CC(F)(F)F)[C@@H]2c1ccc(N2CCC(C(OC)OC)CC2)cc1]

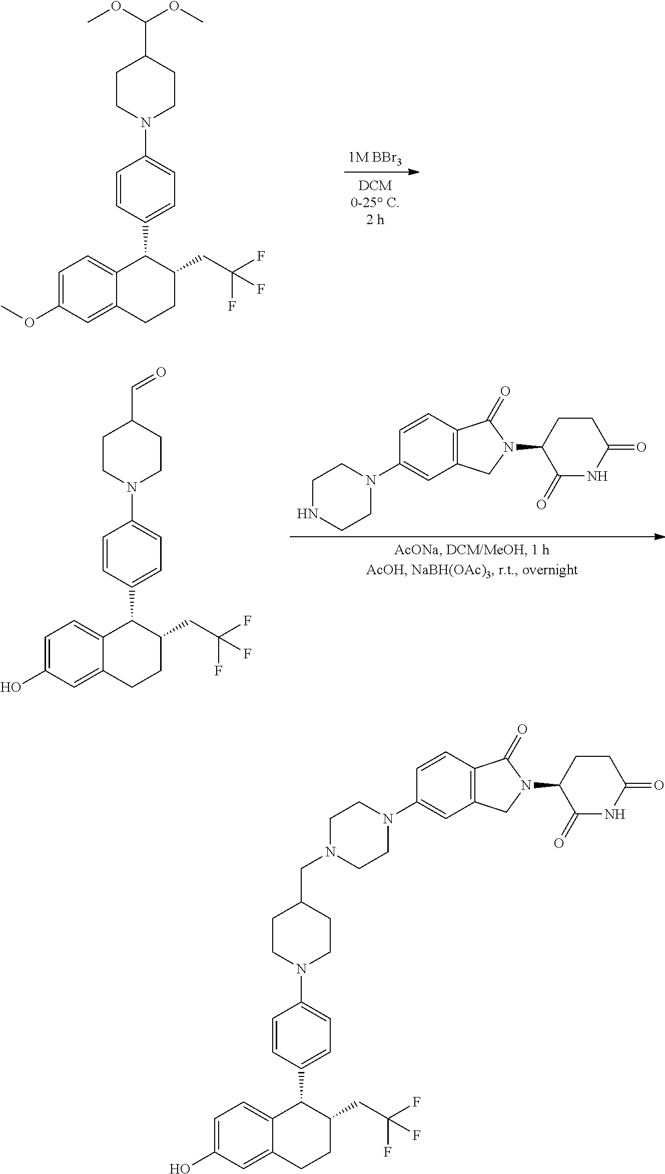

Step 1: Ethyl 2-diethoxyphosphorylacetate (20 g, 89.21 mmol, 17.70 mL, 1 eq) was added into mixture of NaH (7.14 g, 178.42 mmol, 60% purity, 2 eq) in THF (200 mL) under N₂ at 0° C., and the mixture was stirred at 0° C. for 0.5 h under N₂ atmosphere, and then 2,2,2-trifluoroethyl trifluoromethanesulfonate (41.41 g, 178.42 mmol, 24.65 mL, 2 eq) was added in the mixture at 0° C. and was stirred at 25° C. for 16 h. TLC (petroleum ether/ethyl acetate=10:1, R_{f}=0.4) showed new spot was formed. Then the mixture was quenched with H₂O (100 mL), extracted with EtOAc (150 mL). The combined extracts was dried over anhydrous Na₂SO₄, filtered and concentrated to dryness in vacuo to give a residue. The residue was subjected to column chromatography over silica gel (gradient elution: 0-20% ethyl acetate in petroleum ether). The pure fractions were collected and concentrated to dryness in vacuo to give ethyl 2-diethoxyphosphoryl-4,4,4-trifluoro-butanoate (16 g, 52.25 mmol, 58.57% yield) as a colorless oil. LC-MS (ESI⁺) m/z: 307 (M+H)⁺.

Step 2: A mixture of ethyl 2-diethoxyphosphoryl-4,4,4-trifluoro-butanoate (16 g, 52.25 mmol, 1 eq), DBU (7.95 g, 52.25 mmol, 7.87 mL, 1 eq) and LiCl (2.88 g, 67.92 mmol, 1.3 eq) was degassed in CH₃CN (160 mL), and the mixture was stirred at 25° C. for 10 min. and then 2-(3-methoxyphenyl) acetaldehyde (9.42 g, 62.69 mmol, 1.2 eq) was degassed in the mixture, the mixture was stirred at 25° C. for 12 h, TLC (petroleum ether/ethyl acetate=10:1, R_{f}=0.5) showed new spot was formed. Then the mixture was quenched with H₂O (150 Ml), extracted with EtOAc (200 MI). The combined extracts was dried over anhydrous Na₂SO₄, filtered and concentrated to dryness in vacuo to give a residue. The residue was subjected to column chromatography over silica gel (gradient elution: 0˜30% ethyl acetate in petroleum ether). The pure fractions were collected and concentrated to dryness in vacuo to give ethyl 4-(3-methoxyphenyl)-2-(2,2,2-trifluoroethyl) but-2-enoate (5.5 g, 17.75 mmol, 33.43% yield) as a white solid.

Step 3: A mixture of ethyl 4-(3-methoxyphenyl)-2-(2,2,2-trifluoroethyl) but-2-enoate (5.5 g, 17.75 mmol, 1 eq), Pd/C (5.5 g, 0.2 mmol, 10% purity) in THF (50 mL) and purged with H₂ for 3 times, and then the mixture was stirred at 30° C. for 12 hr under H₂ atmosphere, and then the mixture was stirred at 50° C. for 16 hr under H₂ atmosphere. TLC (petroleum ether/ethyl acetate=10:1, R_{f}=0.3). The reaction mixture was filtered and concentrated to dryness in vacuo to give ethyl 4,4,4-trifluoro-2-[2-(3-methoxyphenyl)ethyl]butanoate (5.4 g, 4.93 mmol, 98.18% yield) was obtained as a colorless oil.

Step 4: To a solution of ethyl 4,4,4-trifluoro-2-[2-(3-methoxyphenyl)ethyl]butanoate (5.4 g, 17.75 mmol, 1 eq), NaOH (2 M, 35.49 mL, 4 eq) in THF (20 mL) and MeOH (20 mL) and then the mixture was stirred at 25° C. for 2 hr. TLC (petroleum ether/ethyl acetate=3:1, R_{f}=0.4) showed new spot was formed. The reaction mixture was quenched by addition 70 mL 1 M HCl at 25° C., and then was treated with 20 mL H₂O and extracted with Ethyl acetate (100 mL*3). The combined organic layers were washed with brine (50 mL), dried over Na₂SO₄, The reaction mixture was filtered and concentrated to dryness in vacuo to give 4,4,4-trifluoro-2-[2-(3-methoxyphenyl)ethyl]butanoic acid (4.6 g, 16.65 mmol, 93.84% yield) as a white oil. LC-MS (ESI⁺) m/z: 277 (M+H₂O)⁺.

Step 5: A mixture of 4,4,4-trifluoro-2-[2-(3-methoxyphenyl)ethyl]butanoic acid (2 g, 7.24 mmol, 1 eq), (2,2,2-trifluoroacetyl) 2,2,2-trifluoroacetate (12.16 g, 57.92 mmol, 8.06 mL, 8 eq) in DCM (10 mL) and then the mixture was stirred at 25° C. for 16 hr. TLC (petroleum ether/ethyl acetate==10:1, R_{f}=0.4) showed new spot was formed. The reaction mixture was quenched by addition 50 mL H₂O, and then was treated with 50 mL H₂O and extracted with Ethyl acetate 100 mL (50 mL*2). The organic layers were dried with anhydrous Na₂SO₄, filtered, the residue was subjected to column chromatography over silica gel (gradient elution: 0˜40% ethyl acetate in petroleum ether). The pure fractions were collected and concentrated to dryness in vacuo to give 6-methoxy-2-(2,2,2-trifluoroethyl)tetralin-1-one (1.3 g, 5.03 mmol, 69.53% yield) as a white oil. LC-MS (ESI⁺) m/z: 259 (M+H)⁺.

Step 6: n-BuLi (2.5 M, 3.02 mL, 1.5 eq) was added in the 1-(4-bromophenyl)-4-(dimethoxymethyl)piperidine (2.37 g, 7.55 mmol, 1.5 eq) in THF (15 mL) under N₂ at −78° C. and was stirred at −78° C. for 1 h, and then 6-methoxy-2-(2,2,2-trifluoroethyl)tetralin-1-one (1.3 g, 5.03 mmol, 1 eq) was added in the mixture at −78° C. and was stirred at 25° C. for 12 h under N₂ atmosphere. TLC (petroleum ether/ethyl acetate=5:1, R_{f}=0.4) showed new spot was formed. The reaction mixture was quenched with NH₄Cl 50 mL, and then was treated with 50 mL H₂O and extracted with ethyl acetate 100 mL (50 mL*2), The organic layers were dried with anhydrous Na₂SO₄, filtered, The residue was subjected to column chromatography over silica gel (gradient elution: 0˜10% ethyl acetate in petroleum ether). The pure fractions were collected and concentrated to dryness in vacuo to give 1-[4-[4-(dimethoxymethyl)-1-piperidyl]phenyl]-6-methoxy-2-(2,2,2-trifluoroethyl)tetralin-1-ol (1.6 g, 3.24 mmol, 64.40% yield) as a white oil. LC-MS (ESI⁺) m/z: 494 (M+H)⁺.

Step 7: A mixture of 1-(4-(4-(dimethoxymethyl)piperidin-1-yl)phenyl)-6-methoxy-2-(2,2,2-trifluoroethyl)-1,2,3,4-tetrahydronaphthalen-1-ol (800 mg, 1.62 mmol, 1 eq), 4M MeOH/HCl, and then the mixture was stirred at 40° C. for 12 hr. TLC (petroleum ether/ethyl acetate=5:1, R_{f}=0.6) showed new spot was formed. The reaction mixture was quenched with 50 mL saturated aqueous NaHCO₃, and then was treated with 50 mL H₂O and extracted with ethyl acetate 100 mL (50 mL*2). The organic layers were dried with anhydrous Na₂SO₄, filtered, The reaction mixture was filtered and concentrated to dryness in vacuo to give 4-(dimethoxymethyl)-1-(4-(6-methoxy-2-(2,2,2-trifluoroethyl)-3,4-dihydronaphthalen-1-yl)phenyl)piperidine (700 mg, 1.47 mmol, 90.81% yield) as a white oil.

Step 8: A mixture of 4-(dimethoxymethyl)-1-(4-(6-methoxy-2-(2,2,2-trifluoroethyl)-3,4-dihydronaphthalen-1-yl)phenyl)piperidine (700 mg, 1.47 mmol, 1 eq), Pd/C (700.00 mg, 10% purity) and Pd(OH) 2 (700.00 mg, 20% purity) in THF (5 mL) and MeOH (5 mL) and purged with H₂ for 3 times, and then the mixture was stirred at 60° C. for 12 hr under H₂ atmosphere. LCMS showed 99.309% of desired compound was detected. The reaction was filtered and concentrated to dryness in vacuo to give a white solid. The white solid was subjected by SFC: DAICEL CHIRALPAK AD (250 mm*30 mm, 10 um); mobile phase: [CO₂-EtOH (0.1% NH₃H₂O)]; B %: 25% %, isocratic elution mode; The aqueous phase was lyophilized to dryness to give 4-(dimethoxymethyl)-1-(4-((1S,2S)-6-methoxy-2-(2,2,2-trifluoroethyl)-1,2,3,4-tetrahydronaphthalen-1-yl)phenyl)piperidine (120 mg, 251.28 μmol, 17.07% yield) and 4-(dimethoxymethyl)-1-(4-((1R,2R)-6-methoxy-2-(2,2,2-trifluoroethyl)-1,2,3,4-tetrahydronaphthalen-1-yl)phenyl)piperidine (130 mg, 272.22 μmol, 18.49% yield) was obtained as a white oil. LC-MS (ESI⁺) m/z: 462.2 (M+H)⁺,

Step 9: A mixture of 4-(dimethoxymethyl)-1-(4-((1S,2S)-6-methoxy-2-(2,2,2-trifluoroethyl)-1,2,3,4-tetrahydronaphthalen-1-yl)phenyl)piperidine (60 mg, 125.64 μmol, 1 eq) in DCM (4 mL) was degassed and puged with N₂ for 3 times, and then BBr₃ (1 M, 376.92 μL, 3 eq) was added in the mixture at 0° C. and was stirred at 25° C. for 2 hr under N₂ atmosphere. LCMS showed 95% of desired compound was detected. The reaction mixture was quenched with saturated aqueous NaHCO₃20 mL, and then was treated with 20 mL H₂O and extracted with DCM (25 mL*2). The organic layers were dried with anhydrous Na₂SO₄, filtered, The reaction mixture was filtered and concentrated to dryness in vacuo to give 1-[4-[(1S,2S)-6-hydroxy-2-(2,2,2-trifluoroethyl)tetralin-1-yl]phenyl]piperidine-4-carbaldehyde (50 mg, 119.77 μmol, 95.33% yield) was obtained as a white solid. LC-MS (ESI⁺) m/z: 416 (M+H)⁺.

Step 10: A mixture of (3S)-3-(1-oxo-5-piperazin-1-yl-isoindolin-2-yl)piperidine-2,6-dione (89.93 mg, 179.66 μmol, 1.5 eq, PhSO₃H salt), 1-[4-[(1R,2R)-6-hydroxy-2-(2,2,2-trifluoroethyl)tetralin-1-yl]phenyl]piperidine-4-carbaldehyde (50 mg, 119.77 μmol, 1 eq), sodium acetate (29.48 mg, 359.31 μmol, 3 eq) was added in DCM (4 mL) MeOH (4 mL), and the mixture was stirred at 30° C. for 1 h, and then NaBH(OAc)₃ (50.77 mg, 239.54 μmol, 2 eq) and acetic acid (99.21 mg, 359.31 μmol, 94.49 μL, 3 eq) was added in the mixture, and then the mixture was stirred at 30° C. for 12 hr. TLC (petroleum ether/ethyl acetate=0:1, R_{f}=0.2) showed new spot was formed. The reaction mixture was treated with 30 mL H₂O and extracted with DCM (50 mL*2). The organic layers were dried with anhydrous Na₂SO₄, filtered, the residue was purified by prep HPLC (column: Welch Xtimate C18 150*30 mm*5 um; mobile phase: [water (FA)-ACN]; gradient: 18%-48% B over 7 min). The aqueous phase was lyophilized to dryness to give (3S)-3-[5-[4-[[1-[4-[(1R,2R)-6-hydroxy-2-(2,2,2-trifluoroethyl)tetralin-1-yl]phenyl]-4-piperidyl]methyl]piperazin-1-yl]-1-oxo-isoindolin-2-yl]piperidine-2,6-dione (12.6 mg, 17.26 μmol, 14.41% yield) as a yellow solid. LC-MS (ESI⁺) m/z: 730.2 (M+H)⁺.

HPLC: 94.331%, purity at 220 nm.

¹H NMR (400 MHZ, DMSO-d₆) δ=11.02-10.90 (m, 1H), 9.17-9.03 (m, 1H), 8.18-8.15 (m, 0.303H), 7.56-7.49 (m, 1H), 7.10-7.02 (m, 2H), 6.85-6.78 (m, 4H), 6.66-6.60 (m, 1H), 6.56-6.52 (m, 1H), 6.47-6.42 (m, 1H), 5.01 (br d, J=4.2 Hz, 1H), 4.39-4.28 (m, 1H), 4.25-4.16 (m, 1H), 4.03-3.92 (m, 1H), 3.71-3.57 (m, 2H), 3.31-3.22 (m, 5H), 2.95-2.78 (m, 3H), 2.70-2.57 (m, 3H), 2.41-2.17 (m, 7H), 2.01-1.92 (m, 1H), 1.85-1.51 (m, 7H), 1.27-1.13 (m, 2H).

SFC: retention time, 2.260 min; Area, 93.981%;

### Example 142. Preparation of (I-50) (S)-3-(5-(4-((1-(4-((1R,2S)-6-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

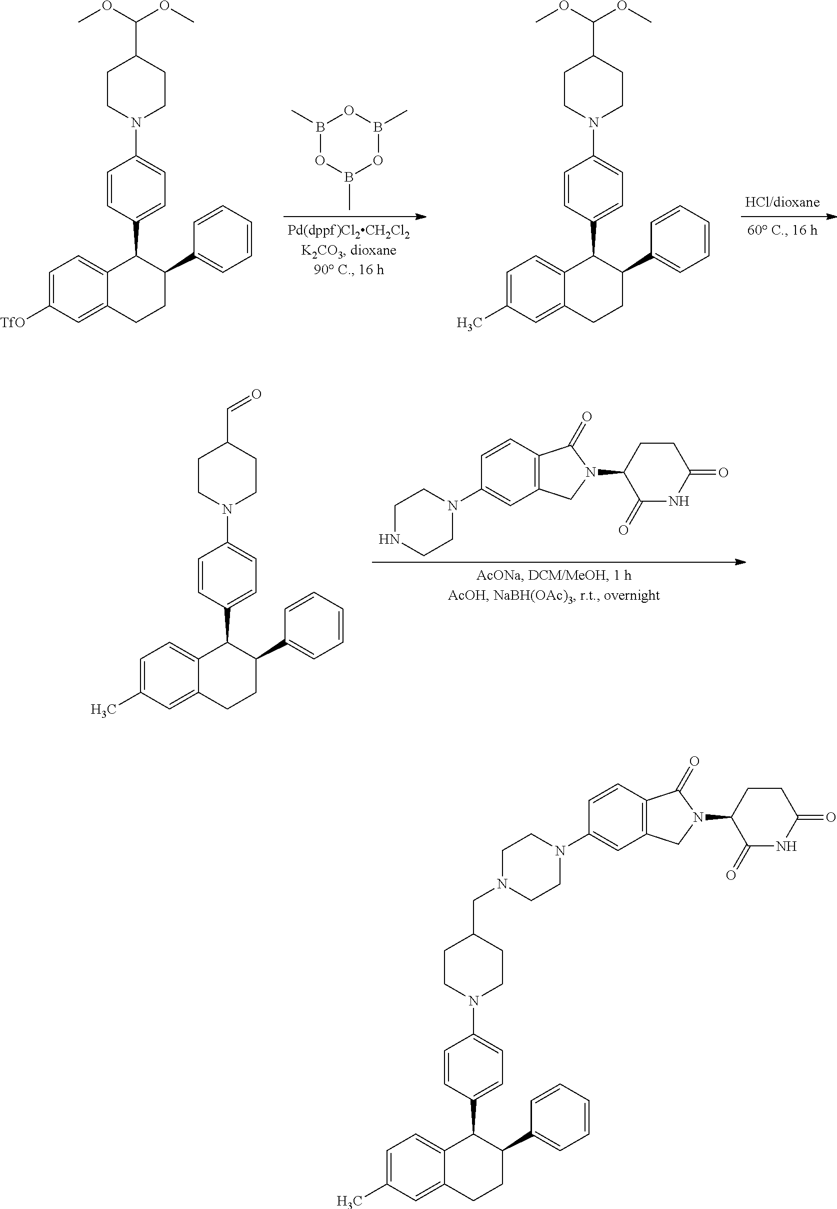

Step 1: To a solution of Pd(dppf) Cl₂·CH₂Cl (42 mg, 0.1 Eq, 50.9 μmol) was added (5R,6S)-5-(4-(4-(dimethoxymethyl)piperidin-1-yl)phenyl)-6-phenyl-5,6,7,8-tetrahydronaphthalen-2-yl]trifluoromethanesulfonate (300 mg, 1 Eq, 509 μmol), 2,4,6-trimethyl-1,3,5,2,4,6-trioxatriborinane (128 mg, 151 μL, 2 Eq, 1.02 mmol) and potassium carbonate (141 mg, 2 Eq. 1.02 mmol) in dioxane (2 mL) at room temperature under N₂ atmosphere. The mixture was stirred at 90° C. for 16 hours. TLC (petroleum ether:ethyl acetate=10:1, UV) showed one main new spot was observed. The reaction was diluted with water (20 mL) and extracted with ethyl acetate (20 mL*2). The combined organic layer were washed with brine (20 mL), dried over Na₂SO₄ and concentrated in vacuum to give crude product as a black oil. The black oil was subjected by flash column (SiO₂, Petroleum ether/Ethyl acetate=100/0 to 85/15). The pure fractions were collected and concentrated in vacuum to give 4-(dimethoxymethyl)-1-(4-((1R, 2S)-6-methyl-2-phenyl-1, 2, 3, 4-tetrahydronaphthalen-1-yl)phenyl)piperidine (162 mg, 255 μmol, 50.2% yield, 72% purity) as a brown solid. LCMS: calc. for C₃₁H₃₇NO₂: 455.28, found: [M+H]⁺456.2.

Step 2: To a solution of 4-(dimethoxymethyl)-1-(4-(1R, 2S)-6-methyl-2-phenyl-1, 2, 3, 4-tetrahydronaphthalen-1-yl)phenyl)piperidine (162 mg, 1 Eq, 356 μmol) in HCl/dioxane (4M, 3 MI) at 60° C. for 16 hour. TLC (petroleum ether:ethyl acetate=10:1, UV) showed one main new spot was observed. The reaction was concentrated to dryness in vacuum to give crude product 1-(4-((1R,2S)-6-methyl-2-phenyl-1, 2, 3, 4-tetrahydronaphthalen-1-yl)phenyl)piperidine-4-carbaldehyde (200 mg, 486 μmol, 99.6% purity) as a pink solid. LCMS: calc. for C₂₉H₃₁: NO: 409.24, found: [M+H]⁺410.1.

Step 3: A mixture of 1-(4-((1R,2S)-6-methyl-2-phenyl-1,2,3,4-tetrahydronaphthalen-1-yl)phenyl)piperidine-4-carbaldehyde (100 mg, 1 Eq, 244 μmol), (R)-3-(1-oxo-5-(piperazin-1-yl)isoindolin-2-yl)piperidine-2,6-dione, TsOH salt (144 mg, 85% purity, 1 Eq, 244 μmol) and sodium acetate (100 mg, 5 Eq, 1.22 mmol) were added in DCM (1.5 mL) and MeOH (1.5 mL) at 25° C. for 1 hour, then added acetic acid (44 mg, 42 μL, 3 Eq, 732 μmol) and sodium cyanoborohydride (31 mg, 29 μL, 2 Eq, 488 μmol), then the mixture was stirred at 25° C. for 16 hours. The reaction was diluted with water (50 mL) and extracted with ethyl acetate (50 mL*2). The combined organic layer were washed with brine (50 mL), dried over Na₂SO₄ and concentrated in vacuum to give crude product as a brown solid. The brown solid was purified by preparative high-performance liquid chromatography.

Condition: Column: Welch xtimate C18 150*30 mm*5 um; A: water (FA), B: CAN, at the beginning: A (74%) and B (26%), at the end: A: (44%) and B (56%), Gradient time (min) 7; 100% B hold time (min) 5.2, Flow rate (ml/min) 25.

The aqueous layer was lyophilized to dryness to give (S)-3-(5-(4-((1-(4-((1R,2S)-6-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (32 mg, 43 μmol, 18% yield, 97.413% purity) as a white solid.

LCMS: calc. for C₄₆H₅₁N₅O₃:721.4, found: [M+H]⁺722.2.

HPLC: 97.413% purity at 220 nm.

¹H NMR (400 MHZ, DMSO-d₆)>=10.95 (s, 1H), 8.15 (s, 0.278H), 7.52 (d, J=8.8 Hz, 1H), 7.18-7.11 (m, 3H), 7.05 (d, J=10.1 Hz, 3H), 6.88-6.83 (m, 3H), 6.74 (d, J=7.9 Hz, 1H), 6.54 (d, J=8.7 Hz, 2H), 6.20 (d, J=8.7 Hz, 2H), 5.05 (dd, J=5.1, 13.3 Hz, 1H), 4.36-4.30 (m, 1H), 4.23-4.17 (m, 2H), 3.51 (br d, J=9.4 Hz, 3H), 3.28 (br d, J=4.1 Hz, 6H), 3.09-2.83 (m, 4H), 2.69-2.53 (m, 3H), 2.39-2.33 (m, 1H), 2.27 (s, 3H), 2.20 (brd, J=7.0 Hz, 2H), 2.14 (br dd, J=6.4, 12.2 Hz, 1H), 2.00-1.92 (m, 1H), 1.75 (br d, J=11.9 Hz, 3H), 1.64 (br d, J=3.3 Hz, 1H), 1.20-1.10 (m, 2H)

SFC: retention time: 3.726 min; Area: 91.718%;

### Example 143. Preparation of (I-40) 3-(4-(4-(1-(4-((1R,2S)-6-hydroxy-2-phenyl-1,2,3,4-tetrahydronaphthalen-1-yl)phenyl)piperidin-4-yl)piperazin-1-yl)phenyl)piperidine-2,6-dione

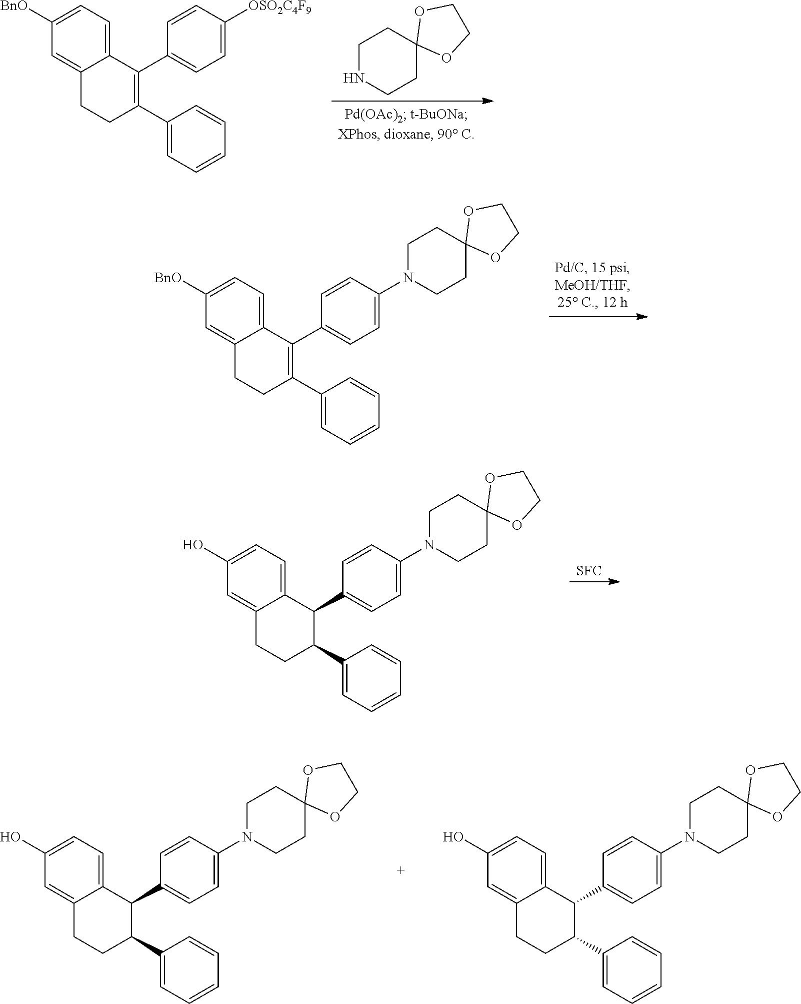

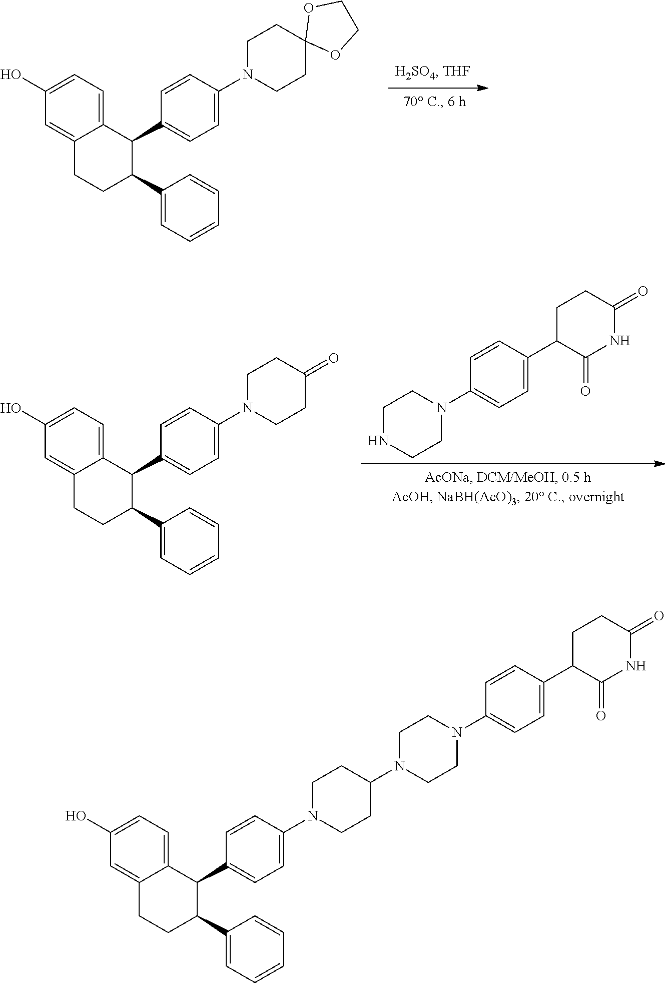 [SMILES: Oc1ccc2c(c1)CC[C@H](c1ccccc1)[C@@H]2c1ccc(N2CCC3(CC2)OCCO3)cc1 ; O=C1CCN(c2ccc([C@@H]3c4ccc(O)cc4CC[C@@H]3c3ccccc3)cc2)CC1 ; O=C1CCC(c2ccc(N3CCNCC3)cc2)C(=O)N1 ; O=C1CCC(c2ccc(N3CCN(C4CCN(c5ccc([C@@H]6c7ccc(O)cc7CC[C@@H]6c6ccccc6)cc5)CC4)CC3)cc2)C(=O)N1]

Step 1: To a solution of 4-(6-(benzyloxy)-2-phenyl-3,4-dihydronaphthalen-1-yl)phenyl 1,1,2,2,3,3,4,4,4-nonafluorobutane-1-sulfonate (1.5 g, 1 Eq, 2.2 mmol) in Toluene (10 mL) was added palladium (II) acetate (49 mg, 0.1 Eq, 0.22 mmol), dicyclohexyl(2′,4′,6′-triisopropyl-[1, l′-biphenyl]-2-yl)phosphane (0.21 g, 0.2 Eq, 0.44 mmol), sodium 2-methylpropan-2-olate (0.63 g, 3 Eq, 6.6 mmol) and 8-Aza-1,4-dioxaspiro[4.5]decane (0.33 g, 0.29 mL, 1.05 Eq, 2.3 mmol) under N₂ balloon. The mixture was stirred under N₂ at 90° C. for 16 h. The reaction mixture was added water 100 mL, and then extracted with ethyl acetate 450 mL (150 mL*3). The combined organic layers were washed with 100 mL water, dried over Na₂SO₄, filtered and concentrated under reduced pressure to give a residue. The residue was purified by column chromatography (SiO₂, petroleum ether/ethyl acetate=100/0 to 50/50) to give 8-(4-(6-(benzyloxy)-2-phenyl-3, 4-dihydronaphthalen-1-yl)phenyl)-1, 4-dioxa-8-azaspiro[4.5]decane (0.6 g, 1 mmol, 40% yield, 84.06% purity) was obtained as a white solid. LC-MS (ESI⁺) m/z: 530.3 (M+H)⁺.

Step 2: A solution of Pd/C (1.2 g, 10% Wt, 0.50 Eq, 1.1 mmol), was added to 8-(4-(6-(benzyloxy)-2-phenyl-3,4-dihydronaphthalen-1-yl)phenyl)-1,4-dioxa-8-azaspiro[4.5]decane (1.2 g, 1 Eq, 2.3 mmol) in THF (2 mL) and MeOH (2 mL). The reaction was degassed and purged with H₂ (4.6 mg, 1 Eq, 2.3 mmol) for 10 mins. The mixture was stirred under H₂ (15 Psi) at 50° C. for 16 h. TLC (petroleum ether:ethyl acetate=1:1,) indicated reactant was consumed completely and one new spots formed. The residue was further separated by SFC (condition: column: DAICEL CHIRALCEL OJ (250 mm*30 mm, 10 um); mobile phase: [0.1% NH₃H₂O ETOH]; B %: 50%-50%) to give desired (5R,6S)-5-(4-(1,4-dioxa-8-azaspiro[4.5]decan-8-yl)phenyl)-6-phenyl-5,6,7,8-tetrahydronaphthalen-2-ol (200 mg, 453 μmol, 20%) as a yellow solid and (5S,6R)-5-(4-(1,4-dioxa-8-azaspiro[4.5]decan-8-yl)phenyl)-6-phenyl-5,6,7,8-tetrahydronaphthalen-2-ol (230 mg, 521 μmol, 23%) as a yellow solid.

Step 3: To a solution of ((5R,6S)-5-(4-(1,4-dioxa-8-azaspiro[4.5]decan-8-yl)phenyl)-6-phenyl-5,6,7,8-tetrahydronaphthalen-2-ol (200 mg, 1 Eq, 453 μmol)) in THF (15 mL) was added Sulfuric acid (25 g, 15 mL, 10% Wt, 56 Eq, 25 mmol). The mixture was stirred at 70° C. for 16 h. The reaction was concentrated under reduced pressure. The residue was purified by flash silica gel chromatography (ISCOR; 40 g SepaFlash® Silica Flash Column, Eluent of 0˜100% ethyl acetate/Petroleum ether gradient @ 80 mL/min) to give the product of give 1-(4-((1R,2S)-6-hydroxy-2-phenyl-1,2,3,4-tetrahydronaphthalen-1-yl)phenyl)piperidin-4-one (200 mg, 503 μmol, 111%) as a light yellow solid. TLC (petroleum ether:ethyl acetate=1:1,) was confirmed.

Step 4: To 1-(4-((1R,2S)-6-hydroxy-2-phenyl-1,2,3,4-tetrahydronaphthalen-1-yl)phenyl)piperidin-4-one (40 mg, 1 Eq, 0.10 mmol), and 3-(4-(piperazin-1-yl)phenyl)piperidine-2,6-dione (41 mg, 1.5 Eq, 0.15 mmol) and titanium tetraisopropoxide (57 mg, 60 μL, 2 Eq, 0.20 mmol) in DCM (2 mL) and MeOH (2 mL) at 0° C. The resulting mixture was stirred for 3 hours at 20° C. Sodium cyanoborohydride (25 mg, 24 μL, 4 Eq, 0.40 mmol) was added to the mixture at 0° C. The resulting mixture was stirred for 16 h at 20° C. The yellow solid was purified by Prep.HPLC (column: Column Phenomenex C18 75*30 mm*3 um

Condition: [water (FA)-ACN]; B %: 20%-60%, 8 min) the pure fractions were collected and the solvent was evaporated under vacuum. The aqueous layer was lyophilized to dryness to give 3-(4-(4-(1-(4-((1R,2S)-6-hydroxy-2-phenyl-1,2,3,4-tetrahydronaphthalen-1-yl)phenyl)piperidin-4-yl)piperazin-1-yl)phenyl)piperidine-2,6-dione (18.9 mg, 28.44 μmol, 37.25% yield, 98.5% purity) was obtained as a white solid.

LCMS: calc. for C₄₂H₄₆N₄O₃:654.36, found: [M+H]⁺655.3.

HPLC: 98.541% purity at 220 nm.

¹H NMR (400 MHZ, DMSO-d₆) δ=10.88-10.73 (m, 1H), 9.22-9.06 (m, 1H), 8.16-8.14 (m, 1H), 7.17 (br s, 3H), 7.08-7.01 (m, 2H), 6.92-6.80 (m, 4H), 6.66-6.46 (m, 5H), 6.26-6.15 (m, 2H), 4.17-4.09 (m, 1H), 3.73 (br dd, J=4.9, 11.0 Hz, 1H), 3.58 (br d, J=10.6 Hz, 2H), 3.40-3.25 (m, 3H), 3.12 (br s, 4H), 3.02-2.89 (m, 2H), 2.68 (br s, 4H), 2.46-2.33 (m, 2H), 2.16-1.96 (m, 3H), 1.91-1.81 (m, 2H), 1.75-1.66 (m, 1H), 1.54-1.42 (m, 2H).

SFC: retention time, 4.742 min, 7.087 min; Area, 50.28%, 49.72%; Stereochemistry was arbitrarily assigned.

### Example 144. Preparation of (I-42) 3-(4-(4-(2-(1-(4-((1S,2R)-6-hydroxy-2-phenyl-1,2,3,4-tetrahydronaphthalen-1-yl)phenyl)piperidin-4-yl)ethyl)piperazin-1-yl)phenyl)piperidine-2,6-dione

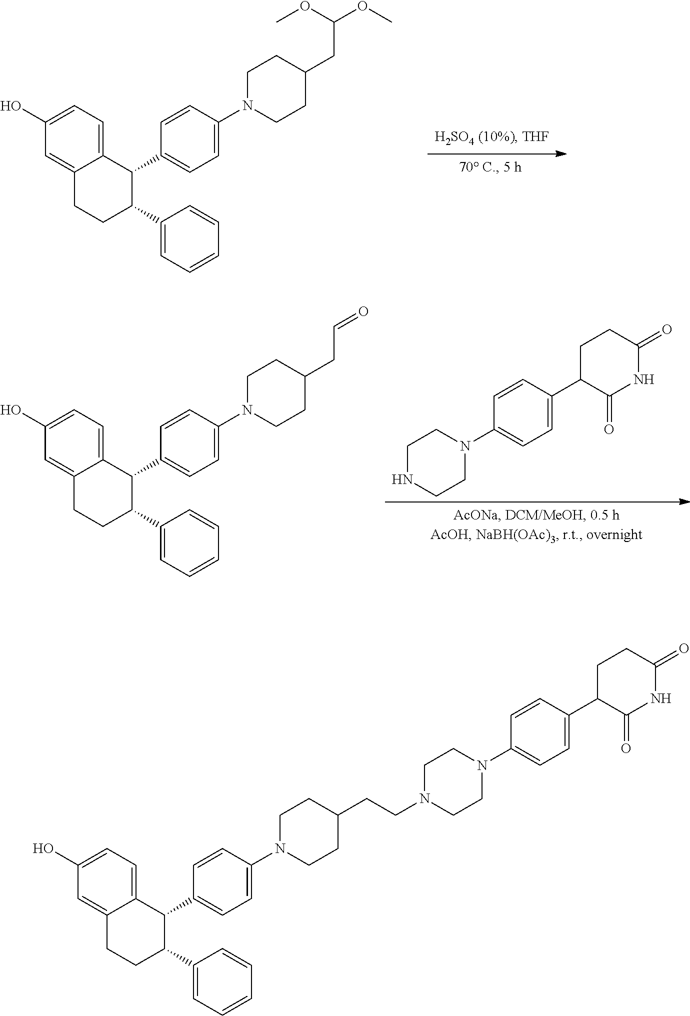 [SMILES: COC(CC1CCN(c2ccc([C@H]3c4ccc(O)cc4CC[C@H]3c3ccccc3)cc2)CC1)OC ; O=CCC1CCN(c2ccc([C@H]3c4ccc(O)cc4CC[C@H]3c3ccccc3)cc2)CC1 ; O=C1CCC(c2ccc(N3CCNCC3)cc2)C(=O)N1 ; O=C1CCC(c2ccc(N3CCN(CCC4CCN(c5ccc([C@H]6c7ccc(O)cc7CC[C@H]6c6ccccc6)cc5)CC4)CC3)cc2)C(=O)N1]

Step 1: To a solution of (5S,6R)-5-(4-(4-(2,2-dimethoxyethyl)piperidin-1-yl)phenyl)-6-phenyl-5,6,7,8-tetrahydronaphthalen-2-ol (300 mg, 1 eq, 636 μmol) in THF (15 mL) was added H₂SO₄ (25 g, 25 mmol, 15 mL, 10% purity, 4 eq). The mixture was stirred at 70° C. for 5 h. The mixture was adjusted to pH=8-9 by progressively adding NaHCO₃. The reaction mixture was quenched by addition 20 mL ethyl acetate at 20° C., and then diluted 20 mL with H₂O and extracted with ethyl acetate (25 mL*2). The combined organic layers were washed with 10 mL brine, dried over Na₂SO₄. The reaction was filtered and concentrated under reduced pressure to give 2-(1-(4-((1S,2R)-6-hydroxy-2-phenyl-1,2,3,4-tetrahydronaphthalen-1-yl)phenyl)piperidin-4-yl) acetaldehyde (400 mg, 940 μmol, crude) was obtained as a yellow solid. LC-MS (ESI⁺) m/z: 426.3 (M+H)⁺

Step 2: A mixture of 2-(1-(4-((1S,2R)-6-hydroxy-2-phenyl-1,2,3,4-tetrahydronaphthalen-1-yl)phenyl)piperidin-4-yl) acetaldehyde (100 mg, 1 Eq, 235 μmol), 3-(4-(piperazin-1-yl)phenyl)piperidine-2,6-dione (64.2 mg, 1 Eq, 235 μmol) and sodium acetate (96.4 mg, 5 Eq, 1.17 mmol) in DCM (2 mL) and MeOH (2 mL) at 20° C. for 0.5 h, then acetic acid (42.3 mg, 40.5 μL, 3 Eq, 705 μmol) at 20° C. for 1 h, then added sodium triacetoxyborohydride (99.6 mg, 2 Eq, 470 μmol) the mixture, and then the mixture was stirred at 25° C. for 16 h. The reaction was concentrated and was purified by prep-HPLC (FA condition: column: Welch Xtimate C18 150*30 mm*5 um; mobile phase: [water (FA)-ACN]; B %: 20%-15%,6 min) to give 3-(4-(4-(2-(1-(4-((1S,2R)-6-hydroxy-2-phenyl-1,2,3,4-tetrahydronaphthalen-1-yl)phenyl)piperidin-4-yl)ethyl)piperazin-1-yl)phenyl)piperidine-2,6-dione (29.8 mg, 43.6 μmol, 18.6%) was obtained as a white solid.

LCMS: calc. for C₄₄H₅₀N₄O₃:682.89, found: [M+H]⁺683.3.

HPLC: 100% purity at 220 nm.

¹H NMR (400 MHz, DMSO-d₆) δ ppm=10.81-10.76 (m, 1H), 8.19-8.16 (m, 1H), 7.18-7.07 (m, 3H), 7.07-7.00 (m, 2H), 6.92-6.85 (m, 2H), 6.84-6.79 (m, 2H), 6.67-6.58 (m, 2H), 6.55-6.45 (m, 3H), 6.23-6.16 (m, 2H), 4.16-4.09 (m, 1H), 3.76-3.67 (m, 1H), 3.49 (br d, J=9.7 Hz, 2H), 3.31-3.24 (m, 1H), 3.14-3.05 (m, 4H), 2.87 (br d, J=6.6 Hz, 2H), 2.67-2.58 (m, 1H), 2.52-2.51 (m, 5H), 2.44-2.42 (m, 1H), 2.48-2.41 (m, 3H), 2.39-2.34 (m, 2H), 2.16-2.05 (m, 2H), 2.02-1.96 (m, 1H), 1.70 (br d, J=11.6 Hz, 2H), 1.43-1.38 (m, 2H), 1.24-1.14 (m, 2H).

SFC: retention time, 6.964 min; Area, 98.794%; Stereochemistry was arbitrarily assigned

### Example 145. Preparation of (I-8) 3-(4-(4-(2-(1-(4-((1R,2S)-6-hydroxy-2-phenyl-1,2,3,4-tetrahydronaphthalen-1-yl)phenyl)piperidin-4-yl)ethyl)piperazin-1-yl)phenyl)piperidine-2,6-dione

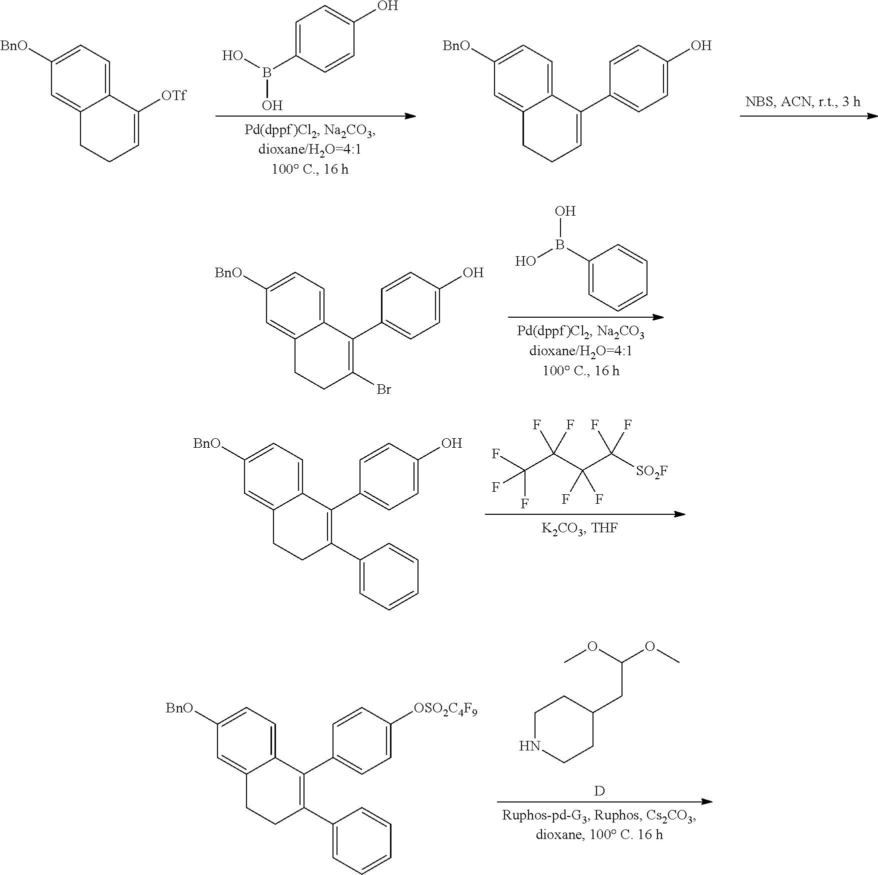

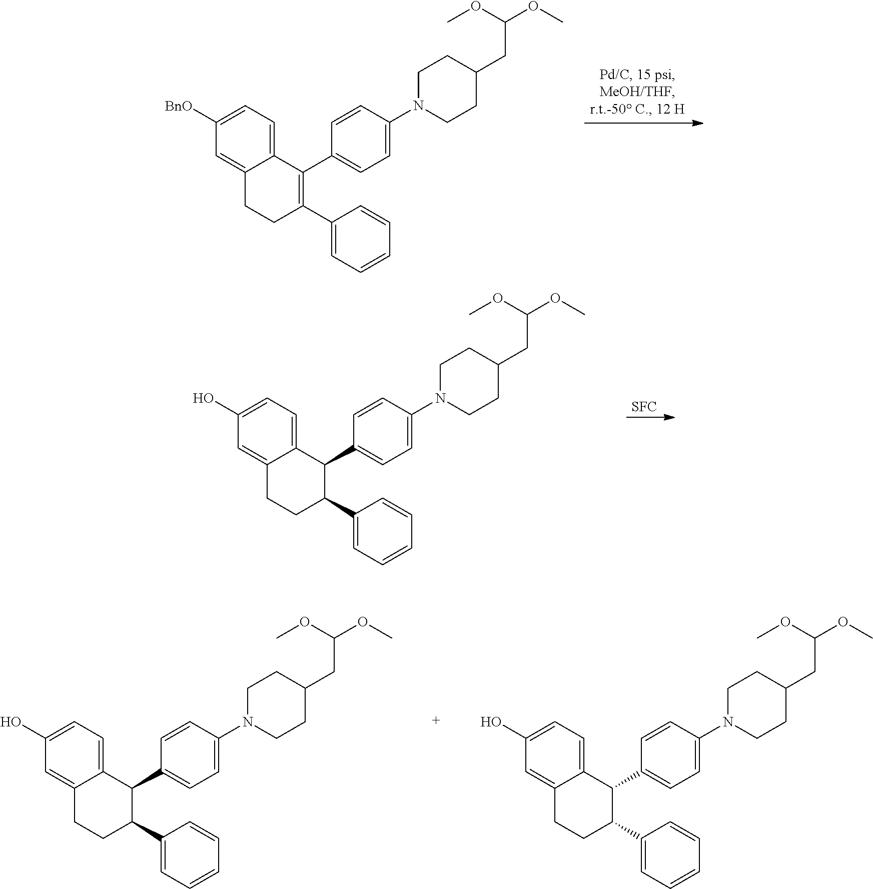 [SMILES: COC(CC1CCN(c2ccc(C3=C(c4ccccc4)CCc4cc(OCc5ccccc5)ccc43)cc2)CC1)OC ; COC(CC1CCN(c2ccc([C@@H]3c4ccc(O)cc4CC[C@@H]3c3ccccc3)cc2)CC1)OC ; "SFC" ; COC(CC1CCN(c2ccc([C@@H]3c4ccc(O)cc4CC[C@@H]3c3ccccc3)cc2)CC1)OC ; COC(CC1CCN(c2ccc([C@H]3c4ccc(O)cc4CC[C@H]3c3ccccc3)cc2)CC1)OC]

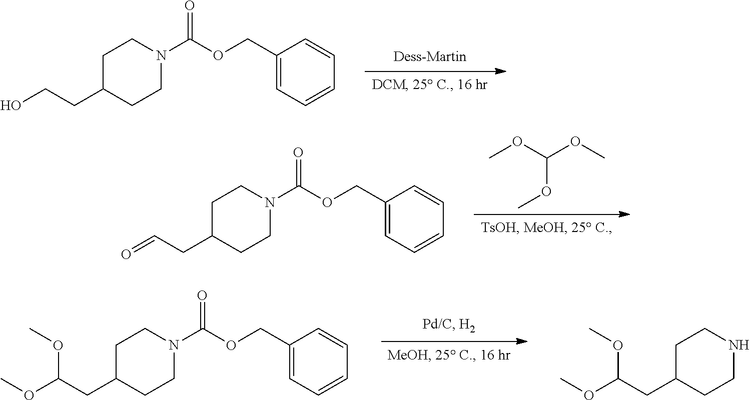

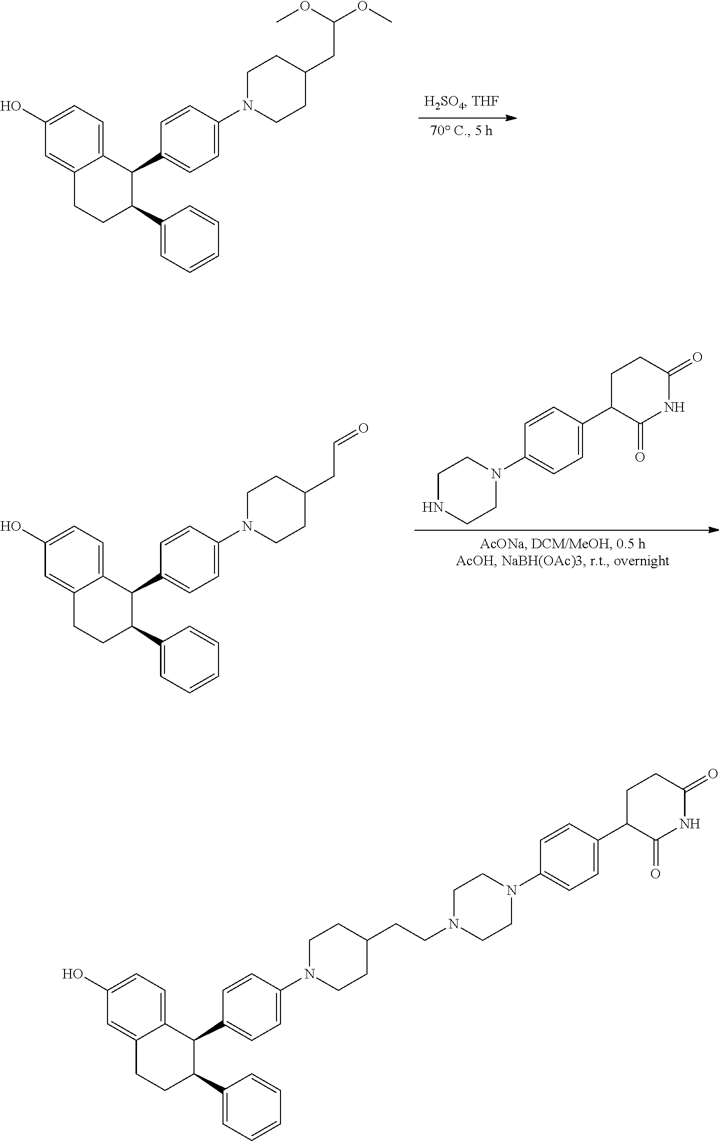 [SMILES: COC(CC1CCN(c2ccc([C@@H]3c4ccc(O)cc4CC[C@@H]3c3ccccc3)cc2)CC1)OC ; O=CCC1CCN(c2ccc([C@@H]3c4ccc(O)cc4CC[C@@H]3c3ccccc3)cc2)CC1 ; O=C1CCC(c2ccc(N3CCNCC3)cc2)C(=O)N1 ; O=C1CCC(c2ccc(N3CCN(CCC4CCN(c5ccc([C@@H]6c7ccc(O)cc7CC[C@@H]6c6ccccc6)cc5)CC4)CC3)cc2)C(=O)N1]

Step 1: A mixture of (6-benzyloxy-3,4-dihydronaphthalen-1-yl) trifluoromethanesμLfonate (10 g, 26.02 mmol, 1 eq), (4-hydroxyphenyl)boronic acid (4.31 g, 31.22 mmol, 1.2 eq), Na₂CO₃ (5.51 g, 52.03 mmol, 2 eq), Pd(dppf) Cl₂ (2.12 g, 2.60 mmol, 0.1 eq) in dioxane (100 mL) and H₂O (20 mL) was degassed and purged with N₂ for 3 times, and then the mixture was stirred at 100° C. for 16 h under N₂ atmosphere. The reaction mixture was added water 500 mL, and then extracted with Ethyl acetate 600 mL (300 mL*3). The combined organic layers were washed with water 300 mL (100 mL*3), dried over Na₂SO₄, filtered and concentrated under reduced pressure to give a residue. The residue was purified by column chromatography (SiO₂, petroleum ether/ethyl acetate=100/0 to 80/20) to give 4-(6-benzyloxy-3, 4-dihydronaphthalen-1-yl) phenol (4.5 g, 10.92 mmol, 42.0% yield, 79.7% purity) was obtained as a white solid. LC-MS (ESI⁺) m/z: 329.0 (M+H)⁺.

Step 2: To a solution of 4-(6-benzyloxy-3,4-dihydronaphthalen-1-yl) phenol (260.32 mg, 792.68 μmol, 1 eq) in ACN (5 mL) was added NBS (126.98 mg, 713.42 μmol, 0.9 eq). The mixture was stirred at 20° C. for 3 h. The residue was concentrated and purified by flash silica gel chromatography (ISCO®; 12 g SepaFlash® Silica Flash Column, Eluent of 0˜50% Ethyl acetate/Petroleum ether gradient @ 30 mL/min). Compound 4-(6-benzyloxy-2-bromo-3, 4-dihydronaphthalen-1-yl) phenol (130 mg, 319.18 μmol, 40.27% yield) was obtained as a white solid. LC-MS (ESI⁺) m/z: 407.0 (M+H)⁺.

Step 3: A mixture of 4-(6-(benzyloxy)-2-bromo-3,4-dihydronaphthalen-1-yl) phenol (5.1 g, 1 Eq, 13 mmol), phenylboronic acid (1.8 g, 1.2 Eq, 15 mmol), Sodium carbonate (2.7 g, 1.5 mL, 2 Eq, 25 mmol), [1, l′-Bis(diphenylphosphino)ferrocene]dichloropalladium(II)Complex with dichloromethane (1.0 g, 0.1 Eq, 1.3 mmol) in 1,4-Dioxane (60 mL) and H₂O (15 mL) was degassed and purged with N₂ for 3 times, and then the mixture was stirred at 100° C. for 16 h under N₂ atmosphere. The reaction mixture was added water 300 mL at 0° C. and extracted with ethyl acetate (200 mL*3). The combined organic layers were washed with brine (40 mL*3), dried over anhydrous sodium sulfate, filtered and concentrated under reduced pressure. The residue was purified by flash silica gel chromatography (ISCOR; 40 g SepaFlash® Silica Flash Column, Eluent of 0˜100% ethyl acetate/petroleum ether gradient @ 80 mL/min) to give the product of 4-(6-(benzyloxy)-2-phenyl-3, 4-dihydronaphthalen-1-yl) phenol (3.9 g, 9.6 mmol, 80%) as a light yellow solid. LC-MS (ESI⁺) m/z: 405.2 (M+H)⁺.

Step 4: To a solution of Nonafluorobutanesulfonyl fluoride (1 g, 0.9 mL, 1 Eq, 5 mmol) and 4-(6-(benzyloxy)-2-phenyl-3,4-dihydronaphthalen-1-yl) phenol (2 g, 1 Eq, 5 mmol) in THF (30 mL) and ACN (30 mL) was added potassium carbonate (1 g, 0.4 mL, 1.5 Eq, 7 mmol). The reaction was stirred at r.t. for 16 h. TLC (petroleum ether:ethyl acetate=10:1, R_{f}=0.5) indicated the starting material was consumed completely and one new spot formed. The reaction mixture was concentrated under reduced pressure. The residue was purified by flash silica gel chromatography (ISCO®; 24 g SepaFlash® Silica Flash Column, Eluent of 0˜10% Ethyl acetate/Petroleum ether gradient @ 45 mL/min). To give the product of 4-(6-(benzyloxy)-2-phenyl-3,4-dihydronaphthalen-1-yl)phenyl 1,1,2,2,3,3,4,4,4-nonafluorobutane-1-sμLfonate (2.9 g, 4.2 mmol, 90%) was obtained as a colorless oil. LC-MS (ESI⁺) m/z: 687.1 (M+H)⁺.

Step 5: A mixture of benzyl 4-(2-hydroxyethyl)piperidine-1-carboxylate (500 mg, 1.90 mmol) in DCM (1 mL) was degassed and purged with N₂ for 3 times, the reaction was added (1,1-diacetoxy-3-oxo-1λ5,2-benziodoxol-1-yl)acetate (805.34 mg, 1.90 mmol, 587.84 μL) at 0° C. and then the mixture was stirred at 25° C. for 3 h under N₂ atmosphere. TLC (Petroleum ether:Ethyl acetate=1:1) indicated reactant was consumed completely and one new spot formed. The reaction was clean according to TLC. The reaction mixture was quenched by addition 5 mL NaHCO₃, the reaction mixture was added filtered and filter liquor was extracted with 15 mL (5 mL*3) DCM. The combined organic layers were washed with dried over [Na₂SO₄], filtered and concentrated under reduced pressure to give a residue. The residue was purified by flash silica gel chromatography (ISCO®; 12 g SepaFlash® Silica Flash Column, Eluent of 0˜40% Ethyl acetate/Petroleum ether gradient @ 25 mL/min) to give benzyl 4-(2-oxoethyl)piperidine-1-carboxylate (200 mg, 765.36 μmol, 40.31% yield, 100.00% purity) was obtained as a yellow oil. LC-MS (ESI⁺) m/z: 262.2 (M+H)⁺.

Step 6: A solution of trimethoxymethane (203.05 mg, 1.91 mmol, 209.76 μL) and benzyl 4-(2-oxoethyl)piperidine-1-carboxylate (100 mg, 382.68 μmol) in MeOH (2 mL) was added 4-methylbenzenesulfonic acid (1.32 mg, 7.65 μmol). Then the mixture was stirred at 25° C. for 1 h. TLC (petroleum ether:ethyl acetate-5:1) showed the starting material was consumed completely and one new spot was formed. The resulting mixture was poured into sat. NaHCO₃ (10 mL), extracted with EtOAc (10 mL*3), and the combined organic phase was dried over Na₂SO₄, filtered and concentrated in vacuum. The residue was purified by flash column silica gel chromatography (ISCO, 4 g SepaFlash silica flash column, eluent of 0-100% ethyl acetate/petroleum ether gradient, 20 mL/min) to give benzyl 4-(2,2-dimethoxyethyl)piperidine-1-carboxylate (70 mg, 227.73 μmol, 59.51% yield) as a white solid.

Step 7: To a solution of benzyl 4-(2,2-dimethoxyethyl)piperidine-1-carboxylate (1.8 g, 5.9 mmol) in methanol (20 mL) was added Pd/C (0.62 g, 10% Wt, 0.59 mmol). The mixture was stirred at 25° C. for 16 h under H₂ (0.12 g, 59 mmol). LCMS showed one new peak was detected. The reaction was filtered and concentrated in vacuum to give 4-(2,2-dimethoxyethyl)piperidine (1 g, 6 mmol, 100% yield). LC-MS (ESI⁺) m/z: 174.1 (M+H)⁺.

Step 8:4-(2,2-dimethoxyethyl)piperidine (0.5 g, 1 Eq, 3 mmol) was added to a mixture suspension of Ruphos-Pd-G3 (0.2 g, 0.1 Eq, 0.3 mmol), Ruphos (0.1 g, 0.1 Eq, 0.3 mmol) and 7-(benzyloxy)-4-(4-((perfluorobutyl) sulfonyl)phenyl)-3-phenyl-1,2-dihydronaphthalene (2 g, 1 Eq, 3 mmol) and cesium carbonate (3 g, 3 Eq, 9 mmol) in Toluene (30 mL). The mixture was stirred at 100° C. for 16 h. The reaction mixture was added water 300 mL at 0° C. and extracted with ethyl acetate (100 mL*3). The combined organic layers were washed with brine (20 mL*2), dried over anhydrous sodium sulfate, filtered and concentrated under reduced pressure. The residue was purified by flash silica gel chromatography (ISCO®; 40 g SepaFlash® Silica Flash Column, Eluent of 0˜100% Ethyl acetate/Petroleum ether gradient @, 45 mL/min) to give the product of 1-(4-(6-(benzyloxy)-2-phenyl-3, 4-dihydronaphthalen-1-yl)phenyl)-4-(2, 2-dimethoxyethyl)piperidine (1 g, 2 mmol, 60% yield) as a yellow solid. LC-MS (ESI⁺) m/z: 560.4 (M+H)⁺.

Step 9: Pd/C (300 mg, 4.19 Eq, 2.82 mmol, 10% wt) was added to a mixture suspension of 1-(4-(6-(benzyloxy)-2-phenyl-3,4-dihydronaphthalen-1-yl)phenyl)-4-(2,2-dimethoxyethyl)piperidine (818 mg, 1 Eq, 672 μmol) in MeOH (4 mL) and THF (20 mL). The suspension was degassed and purged with H₂ (15 psi) for 2 times. The mixture was stirred at 50° C. for 16 h. The reaction was filtrate was concentrated. The residue was further separated by SFC (condition: column: DAICEL CHIRALPAK IG (250 mm*30 mm, 10 um); mobile phase: [0.1% NH₃H₂O ETOH]; B %: 40%-40%, min) to give desired (5R,6S)-5-(4-(4-(2,2-dimethoxyethyl)piperidin-1-yl)phenyl)-6-phenyl-5,6,7,8-tetrahydronaphthalen-2-ol (300 mg, 636 μmol, 43.5%) as a yellow solid and (5S,6R)-5-(4-(4-(2,2-dimethoxyethyl)piperidin-1-yl)phenyl)-6-phenyl-5,6,7,8-tetrahydronaphthalen-2-ol (300 mg, 636 μmol, 43.5%) as a yellow solid. LC-MS (ESI⁺) m/z: 472.3 (M+H)⁺.

Step 10: To a solution of (5R,6S)-5-(4-(4-(2,2-dimethoxyethyl)piperidin-1-yl)phenyl)-6-phenyl-5,6,7,8-tetrahydronaphthalen-2-ol (300 mg, 636 μmol, 1 eq) in THF (15 mL) was added H₂SO₄ (25 g, 25 mmol, 15 mL, 10% purity, 40 eq). The mixture was stirred at 70° C. for 16 h. The mixture was adjusted to pH 8-9 by progressively adding NaHCO₃. The reaction mixture was quenched by addition 20 mL EtOAc at 20° C., and then diluted with 20 mL H₂O and extracted with EtOAc (25 mL*2). The combined organic layers were washed with (10 mL), dried over Na₂SO₄. The reaction was filtered and concentrated under reduced pressure to give 2-(1-(4-((1R, 2S)-6-hydroxy-2-phenyl-1,2,3,4-tetrahydronaphthalen-1-yl)phenyl)piperidin-4-yl) acetaldehyde (400 mg, 940 μmol, crude) was obtained as a yellow solid. LC-MS (ESI⁺) m/z: 426.3 (M+H)⁺.

Step 11: A mixture of 2-(1-(4-((1R,2S)-6-hydroxy-2-phenyl-1,2,3,4-tetrahydronaphthalen-1-yl)phenyl)piperidin-4-yl) acetaldehyde (200 mg, 1 Eq, 470 μmol), 3-(4-(piperazin-1-yl)phenyl)piperidine-2,6-dione (128 mg, 1 Eq, 470 μmol) and sodium acetate (193 mg, 5 Eq, 2.35 mmol) in DCM (3 mL) and MeOH (3 mL) at 20° C. for 0.5 hr, pH=8, then acetic acid (84.7 mg, 81.1 μL, 3 Eq, 1.41 mmol) at 20° C. for 1 hr, pH=6, then added sodium triacetoxyborohydride (199 mg, 2 Eq, 940 μmol) the mixture, and then the mixture was stirred at 25° C. for 16 hr. The reaction was concentrated and was purified by prep-HPLC (FA condition: column: Welch Xtimate C18 150*30 mm*5 um phase: [water (FA)-ACN]; B %: 16%-46%, 6 min) to give 3-(4-(4-(2-(1-(4-((1R,2S)-6-hydroxy-2-phenyl-1,2,3,4-tetrahydronaphthalen-1-yl)phenyl)piperidin-4-yl)ethyl)piperazin-1-yl)phenyl)piperidine-2,6-dione (75.8 mg, 111 μmol, 23.6%) was obtained as a white solid.

LCMS: calc. for C₄₄H₅₀N₄O₃:682.89, found: [M+H]⁺683.3.

HPLC: 99.232% purity at 220 nm.

¹H NMR (400 MHZ, DMSO-d₆) δ ppm=10.80-10.77 (m, 1H), 8.24-8.11 (m, 1H), 7.16-7.10 (m, 3H), 7.06-7.02 (m, 2H), 6.88 (d, J=8.8 Hz, 2H), 6.82 (br d, J=6.4 Hz, 2H), 6.65-6.59 (m, 2H), 6.53-6.47 (m, 3H), 6.19 (d, J=8.6 Hz, 2H), 4.13-4.10 (m, 1H), 3.74-3.69 (m, 1H), 3.51-3.45 (m, 1H), 3.48 (br d, J=10.0 Hz, 1H), 3.30-3.24 (m, 1H), 3.10 (br s, 4H), 3.00-2.86 (m, 2H), 2.67-2.57 (m, 1H), 2.52 (br d, J=1.8 Hz, 4H), 2.48-2.41 (m, 4H), 2.39-2.35 (m, 2H), 2.18-1.94 (m, 4H), 1.69 (br d, J=11.3 Hz, 2H), 1.42 (br s, 2H), 1.23-1.15 (m, 2H).

SFC: retention time, 2.859 min; 3.503 min; Area, 50.675%; 49.305%; Stereochemistry was arbitrarily assigned

### Example 146. Preparation of (I-9) (3S)-3-(5-(4-((1-(4-(2-cyclohexyl-6-hydroxy-1,2,3,4-tetrahydroisoquinolin-1-yl)phenyl)piperidin-4-yl)methyl)piperazin-1-yl)-1-oxoisoindolin-2-yl)piperidine-2,6-dione

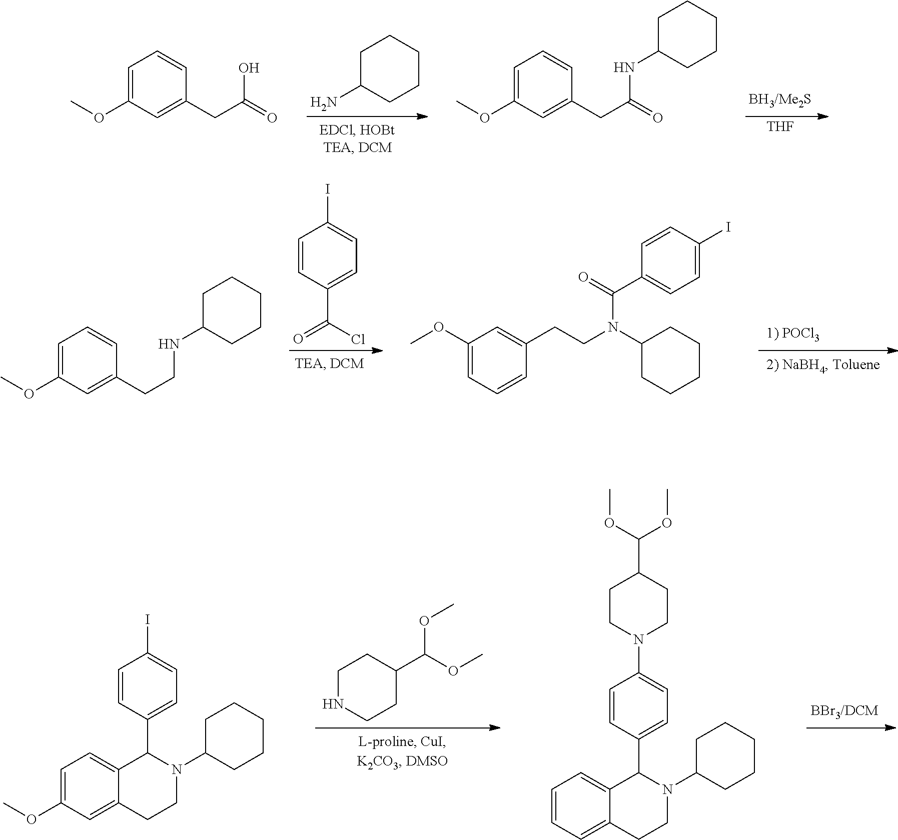

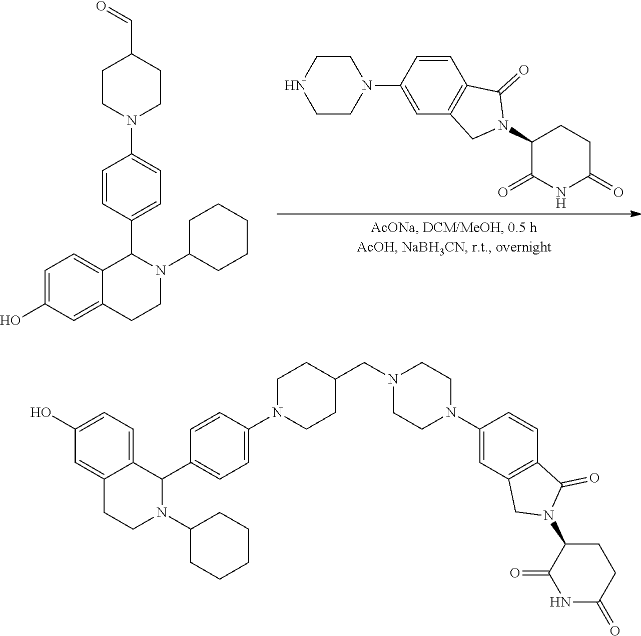 [SMILES: O=CC1CCN(c2ccc(C3c4ccc(O)cc4CCN3C3CCCCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc(C7c8ccc(O)cc8CCN7C7CCCCC7)cc6)CC5)CC4)ccc3C2=O)C(=O)N1]

Step 1: To a solution of 2-(3-methoxyphenyl) acetic acid (20.0 g, 120.36 mmol) and cyclohexanamine (14.3 g, 144.43 mmol, 16.5 mL) in DCM (400.0 mL) was added EDC (28.0 g, 180.53 mmol, 32.0 mL), TEA (36.5 g, 361.07 mmol, 50.26 mL) and HOBt (8.1 g, 60.18 mmol) at 25° C. The mixture was stirred at 25° C. for 2.5 hours. The reaction mixture was concentrated under reduced pressure to give the residue, which was diluted with H₂O (200.0 mL) and extracted with DCM (100.0 mL×3). The combined organic layers were washed with brine (150.0 mL×2), dried over Na₂SO₄, filtered and concentrated under reduced pressure to give a residue, which was purified by column chromatography (SiO₂, Petroleum ether/Ethyl acetate=20/1 to 1/1) to give N-cyclohexyl-2-(3-methoxyphenyl) acetamide (23.3 g, 78.27% yield) as a white solid. LC-MS (ESI⁺) m/z: 248.2 (M+H)⁺.

¹HNMR (500 MHz, DMSO-de): δ 7.90 (d, J=7.5 Hz, 1H), 7.21-7.17 (m, 1H), 7.28-7.22 (m, 1H), 6.82-6.76 (m, 3H), 3.72 (s, 3H), 3.51-3.48 (m, 1H), 3.35-3.33 (m, 2H), 1.72-1.68 (m, 4H), 1.68-1.64 (m, 1H), 1.26-1.12 (m, 5H).

Step 2: To a solution of BH₃/Me₂S (10.0 M, 16.98 mL) in THF (200.0 mL) was added N-cyclohexyl-2-(3-methoxyphenyl) acetamide (21.0 g, 84.91 mmol) in THF (200.0 mL) at 0° C. under N₂. The mixture was stirred at 70° C. for 16 hours under N₂. The reaction mixture was quenched by MeOH (300.0 mL) and reflux for 2 hours. Then the mixture was concentrated under vacuum to get the crude, which was diluted with treated with DCM (300.0 mL) and washed with H₂O (100.0 mL×3) and brine (100.0 mL), dried over Na₂SO₄, filtered and concentrated under reduced pressure to give a residue. The residue was purified by column chromatography (SiO₂, DCM/MeOH=1/0 to 10/1) to give N-(3-methoxyphenethyl)cyclohexanamine (7.4 g, 37.35% yield) as a yellow oil. LC-MS (ESI⁺) m/z: 234.2 (M+H)⁺.

¹HNMR (400 MHz, CHLOROFORM-d): $7.23-7.18 (m, 1H), 6.82-6.74 (m, 3H), 3.79 (s, 3H), 2.92-2.87 (m, 2H), 2.80-2.75 (m, 2H), 2.45-2.40 (m, 1H), 1.88-1.72 (m, 2H), 1.70-1.62 (m, 2H), 1.26-1.03 (m, 6H).

Step 3: To a solution of N-(3-methoxyphenethyl)cyclohexanamine (1.0 g, 4.29 mmol), 4-iodobenzoyl chloride (1.5 g, 5.57 mmol) and TEA (867.3 mg, 8.57 mmol, 1.19 mL) in DCM (10.0 mL) was stirred at 25° C. for 1.5 hours. LCMS showed the desired MS was detected. The mixture was poured into H₂O (20.0 mL), extracted with DCM (20.0 mL×3). The combined organic phase was washed with brine (10.0 mL×2), dried over Na₂SO₄ and filtered. The residue was purified by column chromatography (SiO₂, Petroleum ether/EtOAc=20/1 to 5/1) to give N-cyclohexyl-4-iodo-N-(3-methoxyphenethyl)benzamide (1.9 g, 95.69% yield) as colorless oil. LC-MS (ESI⁺) m/z: 464.1 (M+H)⁺.

¹HNMR (400 MHz, CHLOROFORM-d): δ 7.75 (d, J=8.5 Hz, 2H), 7.23-7.20 (m, 1H), 7.10 (d. J=8.5 Hz, 2H), 6.89-6.77 (m, 3H), 3.81 (s, 3H), 3.53-3.32 (m, 3H), 2.97 (br s, 2H), 1.72-1.70 (m, 2H), 1.60-1.54 (m, 4H), 1.47-1.45 (m, 2H), 1.04 (br s, 2H).

Step 4: To a solution of N-cyclohexyl-4-iodo-N-(3-methoxyphenethyl)benzamide (1.7 g, 3.67 mmol) was added POCl₃ (11.2 g, 73.38 mmol, 6.82 mL) and the mixture was stirred at 120° C. for 16 hours. The mixture was concentrated to get the crude, which was dissolved in Toluene (30.0 mL) and NaBH₄ (1.4 g, 36.69 mmol) was added to the mixture. Then the mixture was stirred at 70° C. for 16 hours. LCMS showed that the desired mass was detected. The mixture was poured into H₂O (30.0 mL), extracted with EtOAc (30.0 mL×3). The combined organic phase was washed with brine (20.0 mL×2), dried over Na₂SO₄ and filtered. The filtrate was concentrated under reduced pressure to get the residue. The residue was purified by column chromatography (SiO₂, Petroleum ether/EtOAc=20/1 to 5/1) to give 2-cyclohexyl-1-(4-iodophenyl)-6-methoxy-1,2,3,4-tetrahydroisoquinoline (1.3 g, 79.21% yield) as a yellow solid. LC-MS (ESI⁺) m/z: 448.1 (M+H)⁺.

¹HNMR (500 MHz, CHLOROFORM-d): δ 7.59 (d, J=8.0 Hz, 2H), 7.05 (d, J=8.5 Hz, 2H), 6.62-6.54 (m, 3H), 4.82 (s, 1H), 3.75 (s, 3H), 3.13-3.10 (m, 1H), 3.02-2.96 (m, 1H), 2.76-2.67 (m, 2H), 2.36-2.31 (m, 1H), 1.93 (d, J=11.5 Hz, 1H), 1.77-1.66 (m, 2H), 1.51-1.50 (m, 1H), 1.43-1.41 (m, 2H), 1.16-1.04 (m, 4H).

Step 5: To a solution of 2-cyclohexyl-1-(4-iodophenyl)-6-methoxy-1,2,3,4-tetrahydroisoquinoline (1.0 g, 2.24 mmol) in DMSO (100.0 mL) was added 4-(dimethoxymethyl)piperidine (533.8 mg, 3.35 mmol), CuI (85.15 mg, 0.45 mmol); pyrrolidine-2-carboxylic acid (102.94 mg, 894.15 μmol) and K₂CO₃ (926.83 mg, 6.71 mmol). The mixture was stirred at 100° C. for 2 hours under N₂. LCMS showed that the desired mass was detected. The mixture was poured into H₂O (20.0 mL), extracted with EtOAC (20.0 mL×3). The combined organic phase was washed with brine (10.0 mL×2), dried over Na₂SO₄ and filtered. The filtrate was concentrated under reduced pressure to get the residue. The residue was purified by column chromatography (SiO₂, Petroleum ether/EtOAc=20/1 to 3/1) to give the product 2-cyclohexyl-1-(4-(4-(dimethoxymethyl)piperidin-1-yl)phenyl)-1,2,3,4-tetrahydroisoquinoline (640.0 mg, 56.82% yield) as a white solid. LC-MS (ESI⁺) m/z: 479.3 (M+H)⁺.

¹H NMR (400 MHz, CHLOROFORM-d) δ 7.12 (d, J=8.4 Hz, 2H), 6.85 (d, J=8.4 Hz, 2H), 6.65-6.59 (m, 2H), 6.57-6.52 (m, 1H), 4.79 (s, 1H), 4.09 (d, J=7.2 Hz, 1H), 3.74 (s, 3H) 3.71-3.64 (m, 2H), 3.37 (s, 6H), 3.17-3.08 (m, 1H), 3.03-2.94 (m, 1H), 2.77-2.60 (m, 4H), 2.47-2.36 (m, 1H), 1.98-1.92 (m, 1H), 1.89-1.81 (m, 2H), 1.78-1.70 (m, 2H), 1.68-1.63 (m, 1H), 1.57-1.51 (m, 2H), 1.48-1.32 (m, 3H), 1.17-1.04 (m, 4H).

Step 6: To a solution of 2-cyclohexyl-1-(4-(4-(dimethoxymethyl)piperidin-1-yl)phenyl)-1,2,3,4-tetrahydroisoquinoline (540.0 mg, 1.13 mmol) in DCM (5.0 mL) was added BBr₃ (1.4 g, 0.5 mL, 5.64 mmol) at −70° C. under N₂. After 10 min, the mixture was stirred at 25° C. for 2 hours. LCMS showed that the desired mass was detected. The mixture was concentrated and purified by prep-TLC (EA) to give the product rac-(R)-1-(4-(2-cyclohexyl-6-hydroxy-1,2,3,4-tetrahydroisoquinolin-1-yl)phenyl)piperidine-4-carbaldehyde (120.0 mg, 25.4% yield). LC-MS (ESI⁺) m/z: 479.3 (M+H)⁺.

Step 7: To a solution of rac-(R)-1-(4-(2-cyclohexyl-6-hydroxy-1,2,3,4-tetrahydroisoquinolin-]-yl)phenyl)piperidine-4-carbaldehyde (100.0 mg, 0.24 mmol), (S)-3-(1-oxo-5-(piperazin-1-yl)isoindolin-2-yl)piperidine-2,6-dione (78.4 mg, 0.24 mmol) in DCM (5.0 mL) and MeOH (5.0 mL) was added AcONa (39.2 mg, 0.48 mmol) and stirred at 25° C. for 20 min, Then AcOH (28.7 mg, 0.48 mmol) and NaBH₃CN (30.0 mg, 0.48 mmol) was added to the mixture and the reaction was stirred for another 3 hours at 25° C. LCMS showed that the desired mass was detected. The mixture was purified by prep-HPLC (Column: Welch Xtimate C18 150*25 mm*5 um; Condition: water (FA)-ACN; Begin B: 18; End B: 38; Gradient Time (min): 11; 100% B Hold Time (min): 2; Flow Rate (ml/min): 25; Detection The mixture was purified by preparative HPLC (water (FA)-ACN as eluents, acidic condition) to give the compound (10 mg, 10% yield) as a yellow solid LC-MS (ESI⁺) m/z: 731.4 (M+H)⁺.

LCMS: calc. for C₄₄H₄₈N₆O₄:731.4.

HPLC: 100% purity at 220 nm.

¹H NMR (400 MHz, CHLOROFORM-d) δ 7.74 (d, J=8.8 Hz, 1H), 7.19-7.09 (m, 2H), 7.00 (dd, J=8.8, 2.0 Hz, 1H), 6.90-6.83 (m, 3H), 6.57-6.52 (m, 2H), 6.47-6.41 (m, 1H), 5.25-5.15 (m, 1H), 4.83 (br s, 1H), 4.45-4.39 (m, 1H), 4.29-4.23 (m, 1H), 3.69 (br d, J=12.0 Hz, 2H), 3.37-3.29 (m, 4H) 3.22-2.56 (m, 10H), 2.54-2.09 (m, 6H), 2.05-1.50 (m, 8H), 1.47-1.31 (m, 4H), 1.19-1.00 (m, 4H).

### Example 147. Preparation of (I-39) 3-[4-[4-[[1-[4-[(1R,2S)-6-hydroxy-2-phenyl-tetralin-1-yl]phenyl]-4-piperidyl]methyl]piperazin-1-yl]phenyl]piperidine-2, 6-dione

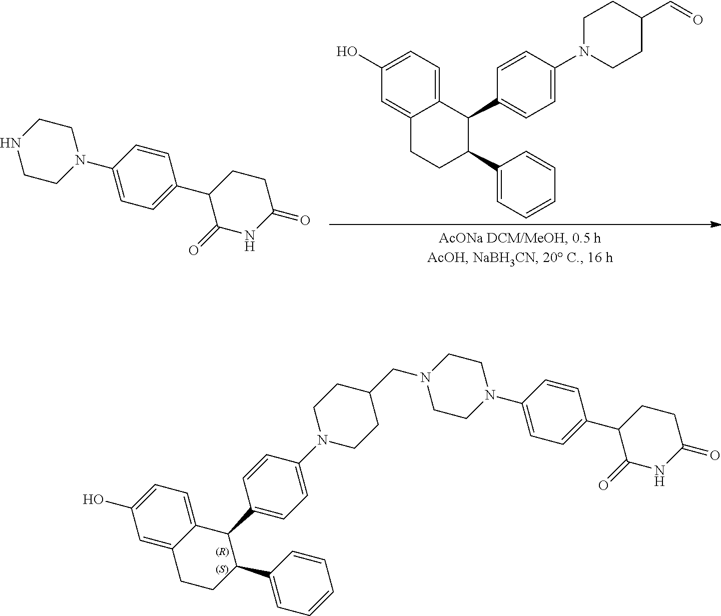

Step 1: A solution of 1-[4-[(1R,2S)-6-hydroxy-2-phenyl-tetralin-1-yl]phenyl]piperidine-4-carbaldehyde (50 mg, 121.5 μmol) 3-(4-piperazin-1-ylphenyl)piperidine-2,6-dione (70 mg, 170 μmol, 3.8 Eq. HCl) and sodium acetate (69.7 mg, 849.8 μmol) in DCM (1.5 mL) and MeOH (1.5 mL) was stirred at 20° C. for 30 mins. Then acetic acid (93.9 mg, 339.9 μmol, 89.4 μL) and NaBH₃CN (21.4 mg, 339.9 μmol) was added. The mixture was stirred at 20° C. for 16 h. LCMS showed 42% desired MS. The reaction was concentrated to dryness in vacuo to give a brown oil. The brown oil was purified by prep-HPLC (column: Boston Green ODS 150*30 mm*5 um; mobile phase: [water (FA)-ACN]; B %: 20%-50%, 6 min). The pure fractions were collected and concentrated in vacuo to remove organic solvent. The aqueous phase was lyophilized to dryness to give the title compound 3-[4-[4-[[1-[4-[(1R,2S)-6-hydroxy-2-phenyl-tetralin-1-yl]phenyl]-4-piperidyl]methyl]piperazin-1-yl]phenyl]piperidine-2, 6-dione (18 mg, 15.5% yield, 0.35 Eq. FA) as a white solid.

LCMS: calc. for C₄₃H₄₈N₄O₃:668.4, found: [M+H]⁺669.3.

HPLC: 97.1% purity at 220 nm

¹H NMR (400 MHZ, DMSO-d₆) δ=10.78 (s, 1H), 9.11 (br s, 1H), 8.15 (s, 1H), 7.19-7.09 (m, 3H), 7.04 (d, J=8.5 Hz, 2H), 6.92-6.80 (m, 4H), 6.64 (d, J=8.3 Hz, 1H), 6.60 (s, 1H), 6.53 (d, J=8.5 Hz, 2H), 6.48 (dd, J=2.3, 8.0 Hz, 1H), 6.20 (d, J=8.5 Hz, 2H), 4.13 (d, J=4.8 Hz, 1H), 3.72 (dd, J=4.9, 11.2 Hz, 1H), 3.51 (br d, J=10.3 Hz, 2H), 3.41-3.22 (m, 5H), 3.10 (br s, 4H), 3.03-2.82 (m, 2H), 2.65-2.52 (m, 4H), 2.23-2.05 (m, 4H), 2.04-1.94 (m, 1H), 1.81-1.54 (m, 4H), 1.23-1.07 (m, 2H)

SFC: retention time, 2.630 min and 3.546 min; Area, 50.17% and 49.83%.

### Example 148. Preparation of (I-11) 3-[4-[4-[[1-[4-[(1S,2R)-6-hydroxy-2-phenyl-tetralin-1-yl]phenyl]-4-piperidyl]methyl]piperazin-1-yl]phenyl]piperidine-2,6-dione

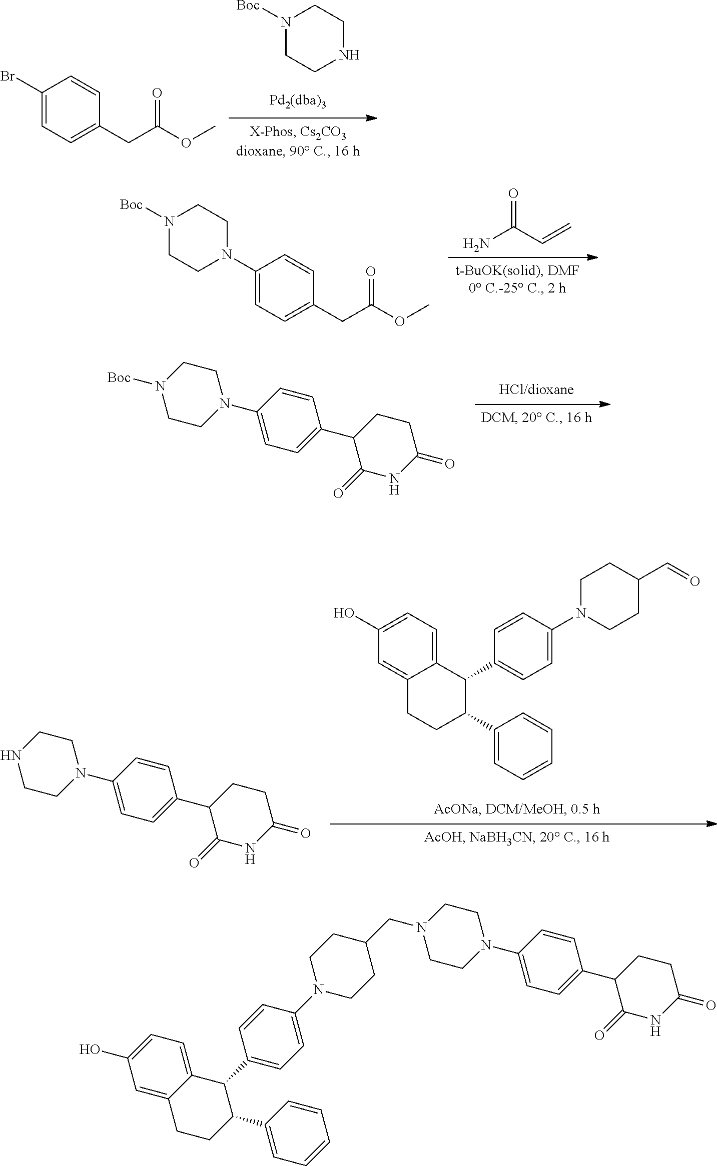

Step 1: A mixture of methyl 2-(4-bromophenyl)acetate (2 g, 8.7 mmol), tert-butyl piperazine-1-carboxylate (1.95 g, 10.5 mmol), cesium carbonate (5.69 g, 17.5 mmol), X-Phos (416.2 mg, 873.1 μmol) and (1E,4E)-1,5-diphenylpenta-1,4-dien-3-one;palladium (399.8 mg, 436.6 μmol) in dioxane (20 mL) was degassed and purged with N₂ for 3 times, and then the mixture was stirred at 90° C. for 16 h under N₂ atmosphere. LC-MS showed 70% of desired MS was detected. The reaction mixture was concentrated under reduced pressure to remove solvent. The residue was diluted with water 100 mL and extracted with Ethyl acetate (25 mL×2). The combined organic layers were washed with brine (15 mL×2), dried over anhydrous Na₂SO₄, filtered and concentrated under reduced pressure to give a yellow oil. The yellow oil was purified by flash silica gel chromatography (ISCOR; 40 g SepaFlash® Silica Flash Column, Eluent of 0˜11% Ethyl acetate/Petroleum ether gradient @ 50 mL/min). The desired fractions were collected and the solvent was concentrated in vacuum to give the product tert-butyl 4-[4-(2-methoxy-2-oxo-ethyl)phenyl]piperazine-1-carboxylate (2.6 g, 7.4 mmol, 84.4% yield, 94.8% purity) as a yellow oil. TLC: petroleum ether:ethyl acetate=5:1, R_{f}=0.5.

LC-MS (ESI⁺) m/z: 335.2 (M+H)⁺.

Step 2: A mixture of tert-butyl 4-[4-(2-methoxy-2-oxo-ethyl)phenyl]piperazine-1-carboxylate (1 g, 3 mmol) in DMF (8 mL) was added potassium 2-methylpropan-2-olate (338.9 mg, 3 mmol) at 0° C., the mixture was degassed and purged with N₂ for 3 times, then prop-2-enamide (214.7 mg, 3 mmol) in DMF (2 mL) was added at 0° C., and then the mixture was stirred at 25° C. for 2 h under N₂ atmosphere. LC-MS showed 14% desired MS was detected. The reaction mixture was poured into aq. NH₄Cl 40 mL at 0° C., and then extracted with Ethyl acetate (15 mL×2). The combined organic layers were washed with brine (15 mL×2), dried over anhydrous Na₂SO₄, filtered and concentrated under reduced pressure to give a green solid. The green solid was purified by flash silica gel chromatography (ISCO®; 25 g SepaFlash® Silica Flash Column, Eluent of 0˜38% Ethyl acetate/Petroleum ether gradient @ 30 mL/min). The desired fractions were collected and the solvent was concentrated in vacuum to give the product tert-butyl 4-[4-(2,6-dioxo-3-piperidyl)phenyl]piperazine-1-carboxylate (170 mg, 428.3 μmol, 14.3% yield, 94.1% purity) as a yellow solid.

TLC: petroleum ether; ethyl acetate=2:1, R_{f}=0.3.

LC-MS (ESI⁺) m/z: 374.2 (M+H)⁺

¹H NMR (400 MHZ, CDCl₃) δ=8.00 (br s, 1H), 7.04 (d, J=8.6 Hz, 2H), 6.85 (d, J=8.7 Hz, 2H), 3.65 (dd, J=5.2, 9.4 Hz, 1H), 3.50 (br t, J=5.0 Hz, 4H), 3.13-3.02 (m, 4H), 2.71-2.52 (m, 2H), 2.25-2.09 (m, 2H), 1.41 (s, 9H).

Step 3: To a solution of tert-butyl 4-[4-(2, 6-dioxo-3-piperidyl)phenyl]piperazine-1-carboxylate (160 mg, 428.4 μmol) in DCM (2 mL) was added HCl/dioxane (2 mL). The mixture was stirred at 20° C. for 16 h. LC-MS showed 86% of desired MS was detected. The reaction was concentrated to dryness in vacuo to give the product 3-(4-piperazin-1-ylphenyl)piperidine-2, 6-dione (175 mg, crude, 3.8 Eq. HCl) as a yellow solid.

LC-MS (ESI⁺) m/z: 274.1 (M+H)⁺.

Step 4: A solution of 1-[4-[(1S,2R)-6-hydroxy-2-phenyl-tetralin-1-yl]phenyl]piperidine-4-carbaldehyde (27 mg, 65.6 μmol), 3-(4-piperazin-1-ylphenyl)piperidine-2,6-dione (50 mg, 121.4 μmol, 3.8 Eq. HCl) and sodium acetate (49.8 mg, 607.5 μmol) in DCM (1.5 mL) and MeOH (1.5 mL) was stirred at 20° C. for 30 mins, Then acetic acid (14.6 mg, 242.8 μmol, 13.9 μL) and sodium cyanoborohydride (15.2 mg, 243 μmol) was added. The mixture was stirred at 20° C. for 16 h. CMS showed 48% desired MS. he reaction was concentrated to dryness in vacuo to give a brown oil. The brown oil was purified by prep-HPLC (column: Boston Green ODS 150*30 mm*5 um; mobile phase: [water (FA)-ACN]; B %: 20%-50%, 6 min). The pure fractions were collected and concentrated in vacuo to remove organic solvent. The aqueous phase was lyophilized to dryness to give the title compound 3-[4-[4-[[1-[4-[(1S,2R)-6-hydroxy-2-phenyl-tetralin-1-yl]phenyl]-4-piperidyl]methyl]piperazin-1-yl]phenyl]piperidine-2,6-dione (13.4 mg, 19.8 μmol, 16.3% yield, 100% purity, 0.15 Eq. FA) as a yellow solid.

LCMS: calc. for C₄₃H₄₈N₄O₃:668.4, found: [M+H]⁺669.5.

HPLC: 99.2% purity at 220 nm

¹H NMR (400 MHZ, DMSO-d₆) δ=10.77 (s, 1H), 9.10 (s, 1H), 8.13 (s, 1H), 7.19-7.08 (m, 3H), 7.04 (br d, J=8.6 Hz, 2H), 6.91-6.79 (m, 4H), 6.67-6.57 (m, 2H), 6.56-6.44 (m, 3H), 6.19 (br d, J=8.5 Hz, 2H), 4.12 (br d, J=4.8 Hz, 1H), 3.71 (dd, J=4.9, 10.9 Hz, 1H), 3.50 (br d, J=10.3 Hz, 2H), 3.30 (s, 6H), 3.10 (br s, 4H), 3.03-2.83 (m, 3H), 2.62 (br s, 2H), 2.26-2.05 (m, 4H), 2.01 (br d, J=4.6 Hz, 1H), 1.81-1.54 (m, 4H), 1.14 (br d, J=9.9 Hz, 2H)

SFC: retention time, 2.528 min and 3.175 min; Area, 48.42% and 51.58%;

### Example 149. Preparation of (I-10) (S)-3-(5-(4-((1-(3,5-difluoro-4-(6-hydroxy-2-phenyl-3,4-dihydronaphthalen-1-yl)phenyl)piperidin-4-yl)methyl)piperazin-1-yl)-1-oxoisoindolin-2-yl)piperidine-2,6-dione

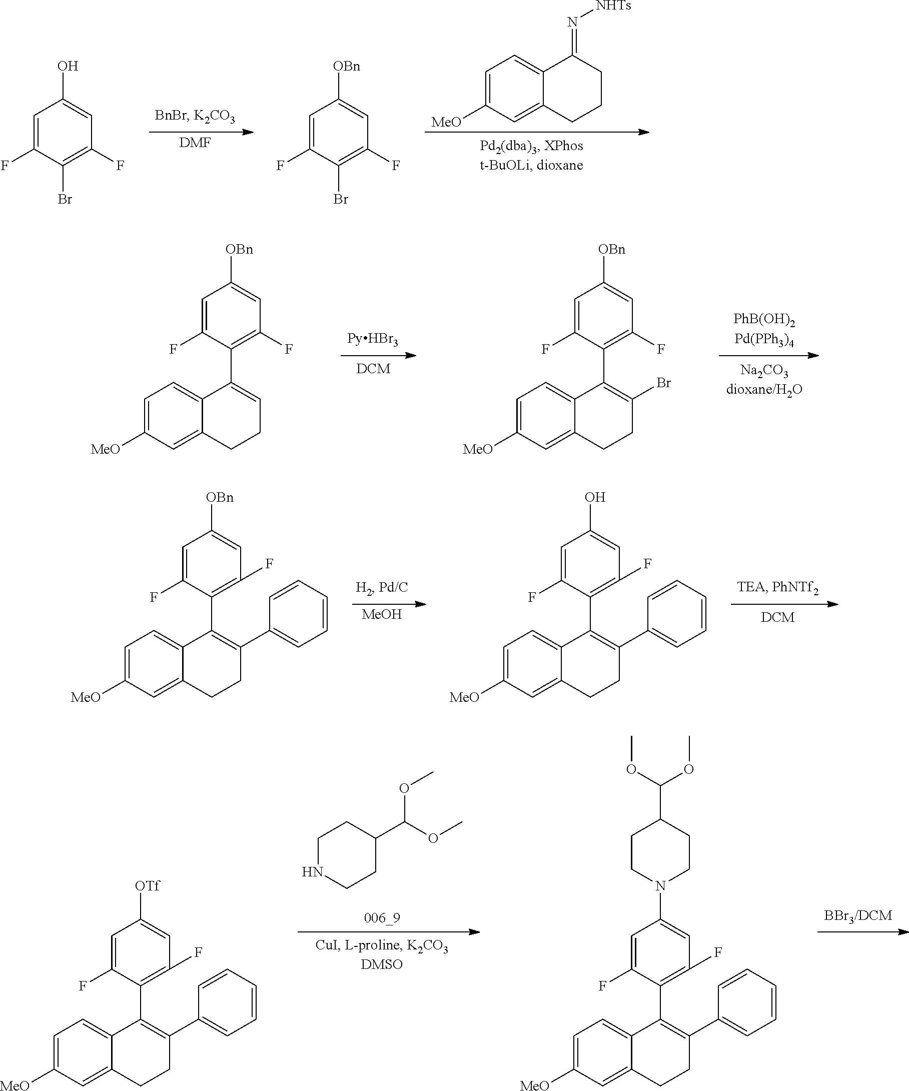

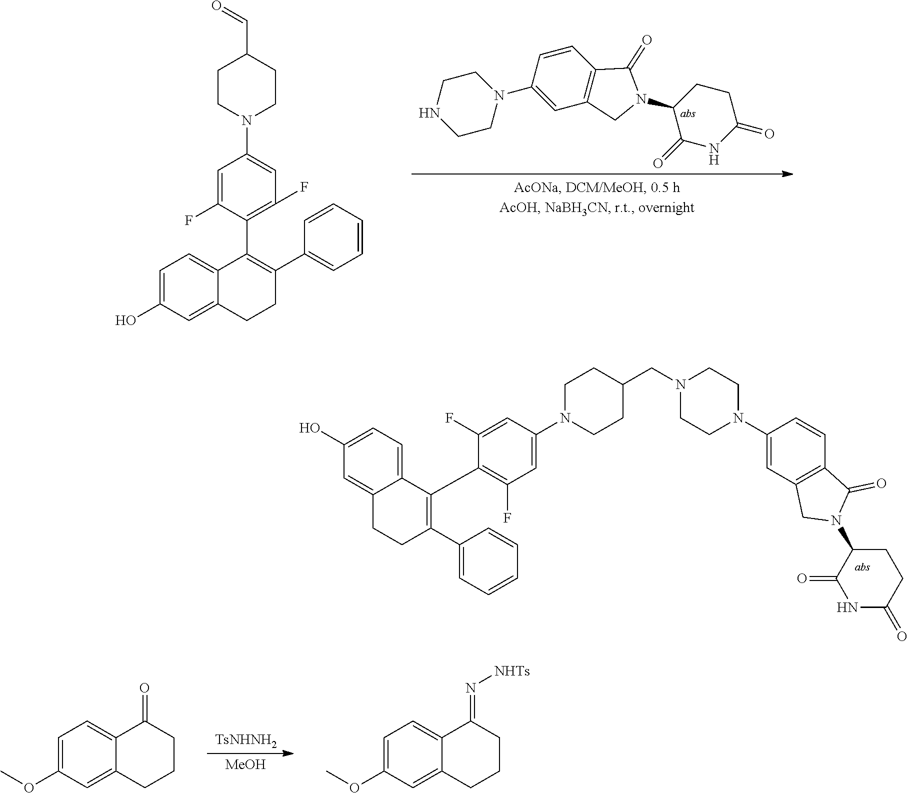

Step 1: To a solution of 4-bromo-3,5-difluoro-phenol (4.0 g, 19.14 mmol) and BnBr (3.9 g, 22.97 mmol, 2.73 mL) in DMF (30 mL) was added K₂CO₃ (7.9 g, 57.42 mmol) at 25° C. The mixture was stirred at 60° C. for 16 hours. The mixture was diluted with H₂O (50 mL) and extracted with EtOAc (30 mL×3). The combined organic layers were washed with brine (40 mL×2), dried over anhydrous Na₂SO₄, filtered and concentrated under reduced pressure to give a residue, which was purified by column chromatography (Petroleum ether/Ethyl acetate=1/0 to 10/1) to give the product of 5-benzyloxy-2-bromo-1,3-difluoro-benzene (5.2 g, 16.52 mmol, 95% purity) as colorless oil.

¹HNMR: (500 MHz, CHLOROFORM-d): δ 7.43-7.33 (m, 5H), 6.64-6.58 (m, 2H), 5.03 (s, 2H)

Step 2: To a solution of 6-methoxytetralin-1-one (8.0 g, 45.40 mmol) in MeOH (200 mL) was added 4-methylbenzenesulfonohydrazide (8.5 g, 45.40 mmol) at 25° C. The mixture was stirred at 25° C. for 16 hours. The mixture was filtered, washed with MeOH (100 mL×3) to give N—[(Z)-(6-methoxytetralin-1-ylidene)amino]-4-methyl-benzenesulfonamide (14.66 g, 90% purity) as a white solid, which was used directly without further purification.

¹HNMR: (500 MHz, CHLOROFORM-d): δ 7.95-7.91 (m, 3H), 7.45-7.42 (m, 1H), 7.32 (d, J=8.0 Hz, 2H), 6.77-6.74 (m, 1H), 6.59 (d, J=2.5 Hz, 1H), 3.80 (s, 3H), 2.71-2.66 (m, 2H), 2.46-2.40 (m, 5H), 1.91-1.85 (m, 2H).

Step 3: To a solution of 5-benzyloxy-2-bromo-1,3-difluoro-benzene (5.0 g, 16.72 mmol) and N—[(Z)-(6-methoxytetralin-1-ylidene)amino]-4-methyl-benzenesulfonamide (8.6 g, 25.07 mmol) in dioxane (200 mL) was added Pd₂(dba)₃ (765.4 mg, 835.81 μmol), XPhos (796.9 mg, 1.67 mmol) and t-BuOLi (2.9 g, 36.78 mmol, 3.32 mL) at 25° C. The mixture was stirred at 100° C. for 16 hours under N₂. The reaction mixture was diluted with H₂O (300 mL) and extracted with EtOAc (200 mL×3). The combined organic layers were washed with brine (500 mL), dried over Na₂SO₄, filtered and concentrated under reduced pressure to give a residue, which was purified by column chromatography (Petroleum ether/Ethyl acetate=1/0 to 10/1) to give 4-(4-benzyloxy-2,6-difluoro-phenyl)-7-methoxy-1,2-dihydronaphthalene (7.6 g, 70% purity) as a white solid.

¹HNMR: (500 MHz, CHLOROFORM-d): δ 7.46-7.34 (m, 5H), 6.76-6.57 (m, 5H), 5.97-5.95 (m, 1H), 5.06 (s, 2H), 3.79 (s, 3H), 2.89-2.85 (m, 2H), 2.47-2.42 (m, 2H).

Step 4: To a solution of 4-(4-benzyloxy-2,6-difluoro-phenyl)-7-methoxy-1,2-dihydronaphthalene (7.0 g, 18.50 mmol) in DCM (100 mL) was added Py·HBr₃ (6.5 g, 20.35 mmol) at 25° C. The mixture was stirred at 25° C. for 5 hours. The reaction mixture diluted with H₂O (200 mL) and extracted with DCM (150 mL×2). The combined organic layers were washed with brine (200 mL), dried over Na₂SO₄, filtered and concentrated under reduced pressure to give a residue, which was purified by flash silica gel chromatography (Petroleum ether/Ethyl acetate=1/0 to 10/1) to give 4-(4-benzyloxy-2,6-difluoro-phenyl)-3-bromo-7-methoxy-1,2-dihydronaphthalene (2.2 g, 26.01% yield) as a yellow solid.

¹HNMR: (400 MHz, CHLOROFORM-d): δ 7.46-7.34 (m, 5H), 6.76-6.57 (m, 5H), 5.07 (s, 2H), 3.78 (s, 3H), 3.05-2.95 (m, 4H).

Step 5: To a solution of 4-(4-benzyloxy-2,6-difluoro-phenyl)-3-bromo-7-methoxy-1,2-dihydronaphthalene (2.2 g, 4.81 mmol) and PhB(OH)₂ (879.9 mg, 7.22 mmol) in dioxane (25 mL) and H₂O (5 mL) was added Na₂CO₃ (1.5 g, 14.43 mmol) and Pd(PPh₃)₄ (555.9 mg, 481.08 μmol) at 25° C. The mixture was stirred at 80° C. for 2 hours under N₂. The reaction mixture was diluted with H₂O (50 mL) and extracted with EtOAc (30 mL×3). The combined organic layers were washed with brine 80 (mL), dried over Na₂SO₄, filtered and concentrated under reduced pressure to give a residue, which was purified by flash silica gel chromatography (Petroleum ether/Ethyl acetate=1/0 to 10/1) to give 4-(4-benzyloxy-2,6-difluoro-phenyl)-7-methoxy-3-phenyl-1,2-dihydronaphthalene (1.55 g, 70.89% yield) as light yellow gum.

¹HNMR: (400 MHz, CHLOROFORM-d): δ 7.45-7.39 (m, 5H), 7.19-7.09 (m, 5H), 6.78 (d, J=2.4 Hz, 1H), 6.64-6.60 (m, 2H), 6.47-6.41 (m, 2H), 4.97 (s, 2H), 3.80 (s, 3H), 3.02-2.98 (m, 2H), 2.84-2.78 (m, 2H).

Step 6: To a solution of 4-(4-benzyloxy-2,6-difluoro-phenyl)-7-methoxy-3-phenyl-1,2-dihydronaphthalene (1.55 g, 3.41 mmol) in MeOH (15 mL) was added Pd/C (1.8 g, 1.71 mmol, 10% purity). The suspension was degassed and purged with H₂ for 3 times. The mixture was stirred under H₂ (15 Psi) at 25° C. for 1 hour. The mixture was filtered and concentrated under reduced pressure to give the residue, which was purified by flash silica gel chromatography (Petroleum ether/Ethyl acetate=1/0 to 3/1) to give 3,5-difluoro-4-(6-methoxy-2-phenyl-3,4-dihydronaphthalen-1-yl) phenol (950.0 mg, 61.16% yield, 80% purity) as a white solid.

¹HNMR: (500 MHz, CHLOROFORM-d): δ 7.19-7.10 (m, 5H), 6.79 (s, 1H), 6.69-6.62 (m, 2H), 6.30 (d, J=8.0 Hz, 2H), 5.16 (s, 1H), 3.81 (s, 3H), 3.02-2.98 (m, 2H), 2.83-2.77 (m, 2H).

Step 7: To a solution of 3,5-difluoro-4-(6-methoxy-2-phenyl-3,4-dihydronaphthalen-1-yl) phenol (950.0 mg, 2.61 mmol) in DCM (20 mL) was added TEA (2.6 g, 26.07 mmol, 3.6 mL) at 25° C. Then PhNTf₂ (1.4 g, 3.91 mmol) in DCM (15 mL) was added at −70° C. The mixture was stirred at −70° C. for 2 hours. The reaction mixture was diluted with H₂O (30 mL) and extracted with DCM (20 mL×3). The combined organic layers were washed with brine (50 mL), dried over Na₂SO₄, filtered and concentrated under reduced pressure to give a residue. The residue was purified by flash silica gel chromatography (Petroleum ether/Ethyl acetate=1/0 to 3/1) to give [3,5-difluoro-4-(6-methoxy-2-phenyl-3,4-dihydronaphthalen-1-yl)phenyl]trifluoromethanesulfonate (515.0 mg, 39.79% yield) was obtained as colorless oil.

¹HNMR: (400 MHZ, CHLOROFORM-d): δ 7.18-7.12 (m, 3H), 7.04 (dd, J=1.7, 7.6 Hz, 2H), 6.83-6.75 (m, 3H), 6.66-6.55 (m, 2H), 3.81 (s, 3H), 3.06-2.99 (m, 2H), 2.86-2.79 (m, 2H).

Step 8: To a solution of 3,5-difluoro-4-(6-methoxy-2-phenyl-3,4-dihydronaphthalen-1-yl)phenyl trifluoromethanesulfonate (50.0 mg, 0.10 mmol) and 4-(dimethoxymethyl)piperidine (19.0 mg, 0.12 mmol) in Toluene (2 mL) was added Cs₂CO₃ (66.0 mg, 2 Eq, 0.20 mmol), Pd(OAc)₂ (1.1 mg, 5.0 μmol) and BINAP (6.3 mg, 10 μmol) at 25° C. The mixture was stirred at 100° C. for 16 hours under N₂. The mixture was concentrated under reduced pressured to give the residue, which was purified by prep-TLC (Petroleum ether/Ethyl acetate=3/1) to give 1-(3,5-difluoro-4-(6-methoxy-2-phenyl-3,4-dihydronaphthalen-1-yl)phenyl)-4-(dimethoxymethyl)piperidine (30.0 mg, 59%) as colorless gum.

¹HNMR: (400 MHz, CHLOROFORM-d): δ 7.17-7.09 (m, 5H), 6.77 (d, J=2.4 Hz, 1H), 6.71 (d, J=8.4 Hz, 1H), 6.64-6.60 (m, 1H), 6.33-6.30 (m, 2H), 4.07 (d, J=7.2 Hz, 1H), 3.80 (s, 3H), 3.64 (d, J=12.4 Hz, 2H), 3.37 (s, 6H), 3.01-2.96 (m, 2H), 2.82-2.77 (m, 2H), 2.73-2.63 (m, 2H), 1.85-1.81 (m, 2H), 1.80-1.71 (m, 1H), 1.41-1.38 (m, 2H).

Step 9: To a solution of 1-(3,5-difluoro-4-(6-methoxy-2-phenyl-3,4-dihydronaphthalen-1-yl)phenyl)-4-(dimethoxymethyl)piperidine (50.0 mg, 99 μmol) in DCM (10 mL) was added BBr₃ (0.12 g, 47 μL, 0.49 mmol) at −70° C. under N₂. After 10 mins, the reaction was stirred at 25° C. for 1 hour. The mixture was quenched by H₂O (15 mL), and adjusted to pH 7˜8 with NH₃H₂O, extracted with DCM (8 mL×3). The combined organic layers were washed with brine (10 mL), dried over Na₂SO₄, filtered and concentrated under reduced pressure to give 1-(3,5-difluoro-4-(6-hydroxy-2-phenyl-3,4-dihydronaphthalen-1-yl)phenyl)piperidine-4-carbaldehyde (67.6 mg, 87%, 57% Purity) as a light yellow solid. LC-MS (ESI⁺) m/z: 492.3 (M+H)⁺.

¹HNMR: (400 MHz, CHLOROFORM-d): δ 9.70 (s, 1H), 7.20-7.06 (m, 5H), 6.72-6.52 (m, 3H), 6.31-6.29 (m, 2H), 4.67 (br s, 1H), 3.72-3.52 (m, 2H), 3.01-2.72 (m, 6H), 2.50-2.37 (m, 1H), 2.06-1.94 (m, 2H), 1.81-1.67 (m, 2H).

Step 10: To a solution of 1-(3,5-difluoro-4-(6-hydroxy-2-phenyl-3,4-dihydronaphthalen-1-yl)phenyl)piperidine-4-carbaldehyde (67.0 mg, 0.15 mmol) and (S)-3-(1-oxo-5-(piperazin-1-yl)isoindolin-2-yl)piperidine-2,6-dione (49.0 mg, 0.15 mmol) in DCM (3 mL) and MeOH (3 mL) was added AcONa (25.0 mg, 0.30 mmol) at 25° C. After 30 min, NaBH₃CN (19.0 mg, 0.30 mmol) and AcOH (18.0 mg, 0.30 mmol) was added to the mixture. The mixture was stirred at 25° C. for 16 hour. The mixture was concentrated under reduced pressured to give the residue, which was purified by prep-HPLC (Column: Welch Xtimate C18 150*25 mm*5 um; Condition: water (FA)-ACN; Begin B: 30; End B: 60; Gradient Time (min): 12; 100% B Hold Time (min): 2; Flow Rate (ml/min): 25; Detection wavelength: 220 nm) to give the compound (18.2 mg, 16%, FA salt) as a white solid.

LCMS: calc. for C₄₅H₄₅F₂N₅O₄: 757.87, found: [M+H]⁺758.3.

HPLC: 100% purity at 254 nm.

SFC: Purity: 100.00%.

¹HNMR: (400 MHz, METHANOL-d₄): δ 8.22 (s, 1H), 7.67-7.65 (m, 1H), 7.14-7.06 (m, 7H), 6.66 (s, 1H), 6.53-6.40 (m, 4H), 5.11 (dd, J=5.2, 13.2 Hz, 1H), 4.42 (d, J=6.0 Hz, 2H), 3.72 (br d, J=12.4 Hz, 2H), 3.49 (br s, 4H), 2.98 (br s, 3H), 2.94-2.89 (m, 3H), 2.80-2.77 (m, 1H), 2.77-2.71 (m, 4H), 2.66 (s, 2H), 2.50-2.42 (m, 1H), 2.18-2.12 (m, 1H), 1.89 (br d, J=12.0 Hz, 3H), 1.43-1.26 (m, 3H).

### Example 150. Preparation of (I-283) (R)-3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione and (I-284) (S)-3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione

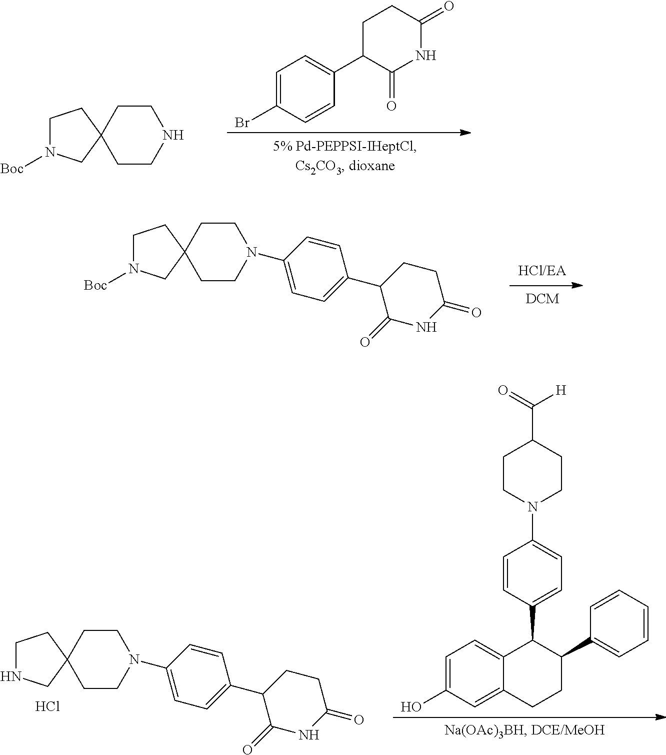 [SMILES: CC(C)(C)OC(=O)N1CCC2(CCNCC2)C1 ; O=C1CCC(c2ccc(Br)cc2)C(=O)N1 ; CC(C)(C)OC(=O)N1CCC2(CCN(c3ccc(C4CCC(=O)NC4=O)cc3)CC2)C1 ; O=C1CCC(c2ccc(N3CCC4(CCNC4)CC3)cc2)C(=O)N1 ; Cl ; O=CC1CCN(c2ccc([C@@H]3c4ccc(O)cc4CC[C@@H]3c3ccccc3)cc2)CC1]

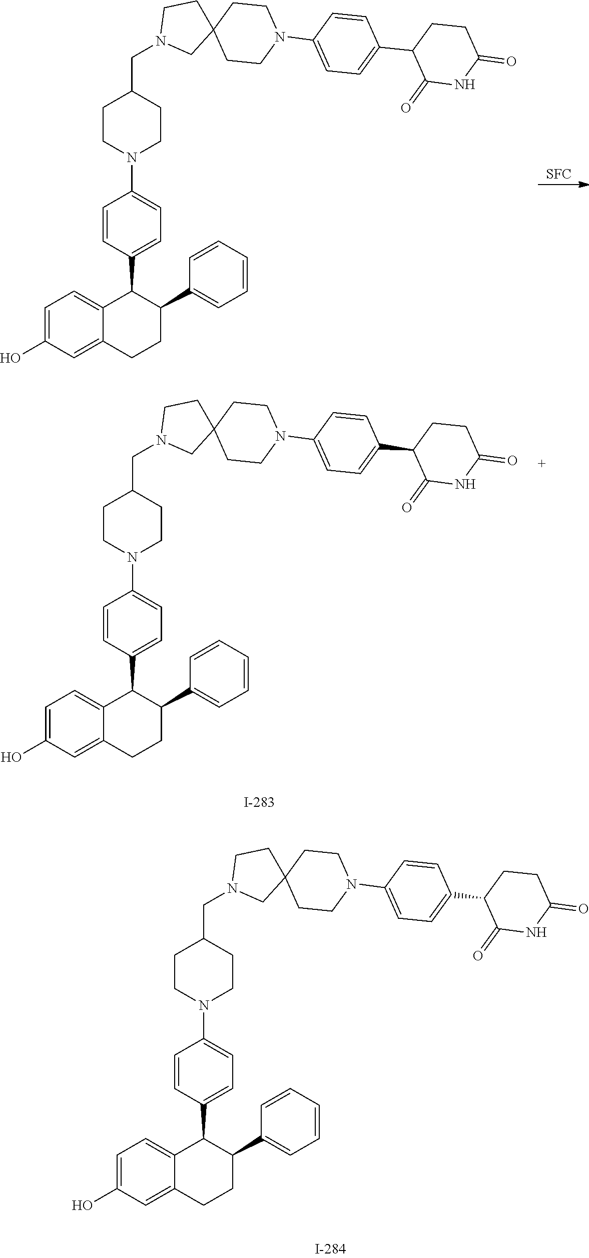 [SMILES: O=C1CCC(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1 ; "SFC" ; O=C1CC[C@H](c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1 ; O=C1CC[C@@H](c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1]

Step 1: To a solution of tert-butyl 2,8-diazaspiro[4.5]decane-2-carboxylate (900.00 mg, 3.74 mmol, 2 eq.) in dioxane (15 mL) was added 3-(4-bromophenyl)piperidine-2,6-dione (502 mg, 1.87 mmol, 1 eq.), Cs₂CO₃ (1.22 g, 3.74 mmol, 2 eq) and 5% Pd-PEPPSI-IHeptCI (182.00 mg, 187 μmol, 0.1 eq). The mixture was stirred at 90° C. under N₂ for 2 hr. LCMS showed the desired ms was found. The mixture was concentrated to give a residue, which was purified by flash silica gel chromatography eluting with EtOAc in PE from 0% to 100% to give tert-butyl 8-(4-(2,6-dioxopiperidin-3-yl)phenyl)-2,8-diazaspiro[4.5]decane-2-carboxylate (440.00 mg, 83.80% purity) as a yellow solid. LC-MS (ESI⁺) m/z: 428.2 (M+H+).

Step 2: To a solution of tert-butyl 8-(4-(2,6-dioxopiperidin-3-yl)phenyl)-2,8-diazaspiro[4.5]decane-2-carboxylate (220.00 mg, 515 μmol, 1 eq.) in DCM (10 mL) was added HCl/EA (1.29 mL, 5.15 mmol, 10 eq.). The mixture was stirred at 20° C. for 1 hr. LCMS showed the desired ms was found. After completion, the mixture was concentrated under reduced pressure to give 3-(4-(2,8-diazaspiro[4.5]decan-8-yl)phenyl)piperidine-2,6-dione hydrochloride (200.00 mg, crude) as a yellow solid without purification.

Step 3: To a solution of 3-(4-(2,8-diazaspiro[4.5]decan-8-yl)phenyl)piperidine-2,6-dione hydrochloride (200.00 mg, 611.00 μmol, 1 eq.) in DCE (10 mL) and MeOH (5 mL) was added 1-(4-((1R,2S)-6-hydroxy-2-phenyl-1,2,3,4-tetrahydronaphthalen-1-yl)phenyl)piperidine-4-carbaldehyde (126.00 mg, 305.00 μmol, 0.5 eq.) and NaBH(OAc)₃ (1.55 g, 7.33 mmol, 12 eq.). The mixture was stirred at 20° C. for 1 hr. The mixture was purified by Prep-HPLC (Column: Boston Green ODS 150 $30 mm+5 um; Condition water (FA)-CAN, B %: 13%-43%, 14 min) to give 3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (130.00 mg, 180 μmol, 29.4% yield, 100% purity) as a yellow solid. LC-MS (ESI⁺) m/z: 723.4 (M+H)⁺.

Step 4:3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (130.00 mg, 180.00 μmol, 1 eq.) was separated by chiral SFC (column: DAICEL CHIRALPAK IE 250 mm #30 mm, 10 μm); mobile phase: [MeOH-ACN]; B %: 50%-50%; Flow Rate: 80 ml/min; peak 1:3.484 min; peak 2:4.629 min) to yield peak 1 and peak 2.

Peak 1 was concentrated under reduced pressure to yield compound (R)-3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (4.0 mg, 6 μmol, 3% yield, 100% purity) as a white solid. LC-MS (ESI⁺) m/z: 723.4 (M+H)⁺,

LCMS: calc. for C₄₇H₅₄N₄O₃:722.97, found: [M+H]⁺723.4.

HPLC: 100% purity at 220 nm.

¹H NMR: 1H NMR (500 MHz, DMSO-d₆) δ=10.76 (s, 1H), 9.09 (s, 1H), 8.25 (br d, J=1.4 Hz, 1H), 7.14-7.11 (m, 3H), 7.02 (d, J=8.7 Hz, 2H), 6.88 (d, J=8.9 Hz, 2H), 6.83 (d, J=6.7 Hz, 2H), 6.64 (d, J=8.4 Hz, 1H), 6.59 (s, 1H), 6.52 (d, J=8.7 Hz, 2H), 6.48-6.46 (m, 1H), 6.19 (d, J=8.7 Hz, 2H), 4.12 (d, J=5.0 Hz, 1H), 3.72-3.69 (m, 1H), 3.51-3.48 (m, 2H), 3.13-3.03 (m, 5H), 2.97-2.90 (m, 2H), 2.43 (br d, J=0.6 Hz, 1H), 2.33 (s, 2H), 2.22 (br d, J=7.5 Hz, 2H), 2.13-1.98 (m, 4H), 1.76-1.67 (m, 4H), 1.63-1.54 (m, 8H), 1.51-1.47 (m, 1H), 1.19-1.08 (m, 3H).

Peak 2 was concentrated under reduced pressure to yield compound (S)-3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (6.3 mg, 8.7 μmol, 4.8% yield, 100% purity) as a white solid. LC-MS (ESI⁺) m/z: 723.4 (M+H)⁺.

LCMS: calc. for C₄₇H₅₄N₄O₃: 722.97, found: [M+H]⁺723.4.

HPLC: 100% purity at 220 nm.

¹H NMR: 1H NMR (500 MHz, DMSO-d₆) δ=10.76 (s, 1H), 9.09 (s, 1H), 8.15 (s, 1H), 7.15-7.11 (m, 3H), 7.03 (d, J=8.5 Hz, 2H), 6.89 (d, J=8.7 Hz, 2H), 6.83 (br d, J=6.9 Hz, 2H), 6.65 (d. J=8.2 Hz, 1H), 6.60 (s, 1H), 6.53 (d, J=8.7 Hz, 2H), 6.49-6.47 (m, 1H), 6.20 (d, J=8.7 Hz, 2H), 4.13 (d, J=4.7 Hz, 1H), 3.74-3.70 (m, 1H), 3.52-3.48 (m, 2H), 3.16-3.05 (m, 5H), 2.98-2.88 (m, 2H), 2.60 (br d, J=5.2 Hz, 1H), 2.46-2.44 (m, 2H), 2.31 (br s, 2H), 2.14-1.99 (m, 4H), 1.79-1.69 (m, 4H), 1.61 (br s, 8H), 1.55-1.51 (m, 1H), 1.24-1.10 (m, 3H).

### Example 151. Preparation of (I-288) 1-(4-((R)-7-((1-(4-((1R,2S)-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)dihydropyrimidine-2,4 (1H,3H)-dione

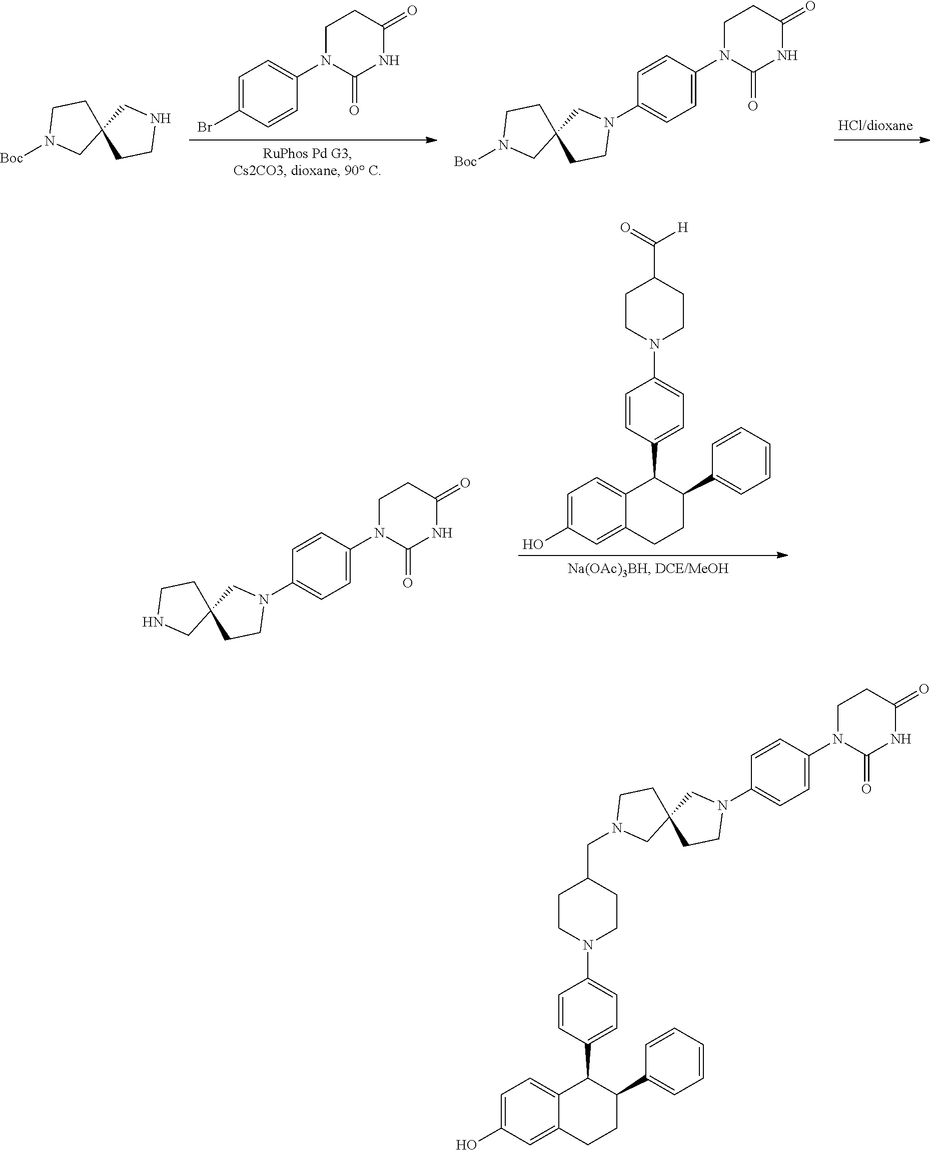 [SMILES: CC(C)(C)OC(=O)N1CC[C@@]2(CCNC2)C1 ; O=C1CCN(c2ccc(Br)cc2)C(=O)N1 ; CC(C)(C)OC(=O)N1CC[C@@]2(CCN(c3ccc(N4CCC(=O)NC4=O)cc3)C2)C1 ; O=C1CCN(c2ccc(N3CC[C@@]4(CCNC4)C3)cc2)C(=O)N1 ; O=CC1CCN(c2ccc([C@@H]3c4ccc(O)cc4CC[C@@H]3c3ccccc3)cc2)CC1 ; O=C1CCN(c2ccc(N3CC[C@@]4(CCN(CC5CCN(c6ccc([C@@H]7c8ccc(O)cc8CC[C@@H]7c7ccccc7)cc6)CC5)C4)C3)cc2)C(=O)N1]

Step 1: To a solution of tert-butyl (R)-2,7-diazaspiro[4.4]nonane-2-carboxylate (500 mg, 2.21 mmol, 2.0 eq), 1-(4-bromophenyl)dihydropyrimidine-2,4 (1H,3H)-dione (297.26 mg, 1.10 mmol, 1.0 eq), Cs₂CO₃ (719.84 mg, 2.21 mmol, 2.0 eq) and RuPhos Pd G3 (92.39 mg, 110.47 μmol, 0.1 eq) in dioxane (10 mL) was bubbled with N₂ for 1 min. Then the mixture was stirred at 90° C. for 16 hours. LCMS showed the starting material was consumed completely and a major peak with desired MS was detected. The reaction mixture was concentrated to get a residue. The residue was purified by column chromatography (SiO₂, Petroleum ether/Ethyl acetate=1/0 to 0/1) to give tert-butyl (R)-7-(4-(2,4-dioxotetrahydropyrimidin-1 (2H)-yl)phenyl)-2,7-diazaspiro[4.4]nonane-2-carboxylate (300 mg, 665.87 μmol, 60.28% yield) as a white solid. LC-MS (ESI⁺) m/z: 415.2 (M+H)⁺, ¹H NMR (400 MHZ, DMSO-d₆) δ ppm 10.20 (s, 1H), 7.09 (d, J=8.82 Hz, 2H), 6.50 (d, J=8.82 Hz, 2H), 3.62-3.70 (m, 2H), 3.32-3.39 (m, 3H), 3.28-3.31 (m, 1H), 3.15-3.27 (m, 4H), 2.67 (t, J=6.74 Hz, 2H), 1.80-1.98 (m, 4H), 1.40 (br d, J=5.60 Hz, 9H).

Step 2: A solution of tert-butyl (R)-7-(4-(2,4-dioxotetrahydropyrimidin-1 (2H)-yl)phenyl)-2,7-diazaspiro[4.4]nonane-2-carboxylate (120.00 mg, 289.51 μmol, 1.0 eq) in DCM (10 mL) was added dropwise into a solution HCl/Dioxane (4 M, 5 mL, 69.08 eq) at 20° C. for 2 h. LCMS showed that the starting material was consumed and the desired product was detected. The reaction mixture was concentrated to get (R)-1-(4-(2,7-diazaspiro[4.4]nonan-2-yl)phenyl)dihydropyrimidine-2,4 (1H,3H)-dione (90 mg, crude) was obtained as a white solid. LC-MS (ESI⁺) m/z: 315.2 (M+H)⁺.

Step 3: To a solution of (R)-1-(4-(2,7-diazaspiro[4.4]nonan-2-yl)phenyl)dihydropyrimidine-2,4 (1H,3H)-dione (90 mg, 256.53 μmol, 1.0 eq, HCl) in DCE (15 mL) and MeOH (3 mL) was added 1-(4-((1R,2S)-6-hydroxy-2-phenyl-1,2,3,4-tetrahydronaphthalen-1-yl)phenyl)piperidine-4-carbaldehyde (63.34 mg, 153.92 μmol, 0.6 eq) and NaBH(OAc); (271.84 mg, 1.28 mmol, 5.0 eq) at 20° C. Then the mixture was stirred at 20° C. for 2 hours. LCMS showed a major peak with desired MS was detected. The reaction mixture was concentrated to get a residue. The residue was purified by prep-HPLC (column: C18 150×30 mm; mobile phase: [water(NH₄HCO₃)-ACN]; gradient: 80%-100% B over 11 min) to give 1-(4-((R)-7-((1-(4-((1R,2S)-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)dihydropyrimidine-2,4 (1H,3H)-dione (12.5 mg, 17.61 μmol, 6.86% yield) as a white solid. LC-MS (ESI⁺) m/z: 710.4 (M+H)⁺.

LCMS: calc. for C₄₅H₅₁N₅O₃: 709.94, found: [M+H]⁺710.4.

HPLC: 100.00% purity at 220 nm

¹H NMR (400 MHZ, DMSO-d₆) δ ppm 10.20 (s, 1H), 9.09 (s, 1H), 7.06-7.16 (m, 5H), 6.82 (br d, J=6.56 Hz, 2H), 6.58-6.65 (m, 2H), 6.45-6.53 (m, 5H), 6.14-6.24 (m, 2H), 4.12 (d, J=5.13 Hz, 1H), 3.65 (t, J=6.68 Hz, 2H), 3.45-3.52 (m, 2H), 3.20-3.28 (m, 4H), 3.09-3.15 (m, 1H), 2.88-2.98 (m, 2H), 2.54-2.75 (m, 4H), 2.23-2.41 (m, 4H), 1.85-2.20 (m, 4H), 1.67-1.78 (m, 5H), 1.45-1.53 (m, 1H), 1.07-1.27 (m, 3H).

### Example 152. Preparation of (I-280) 3-(4-((R)-7-((1-(4-((1R,2S)-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)piperidine-2,6-dione

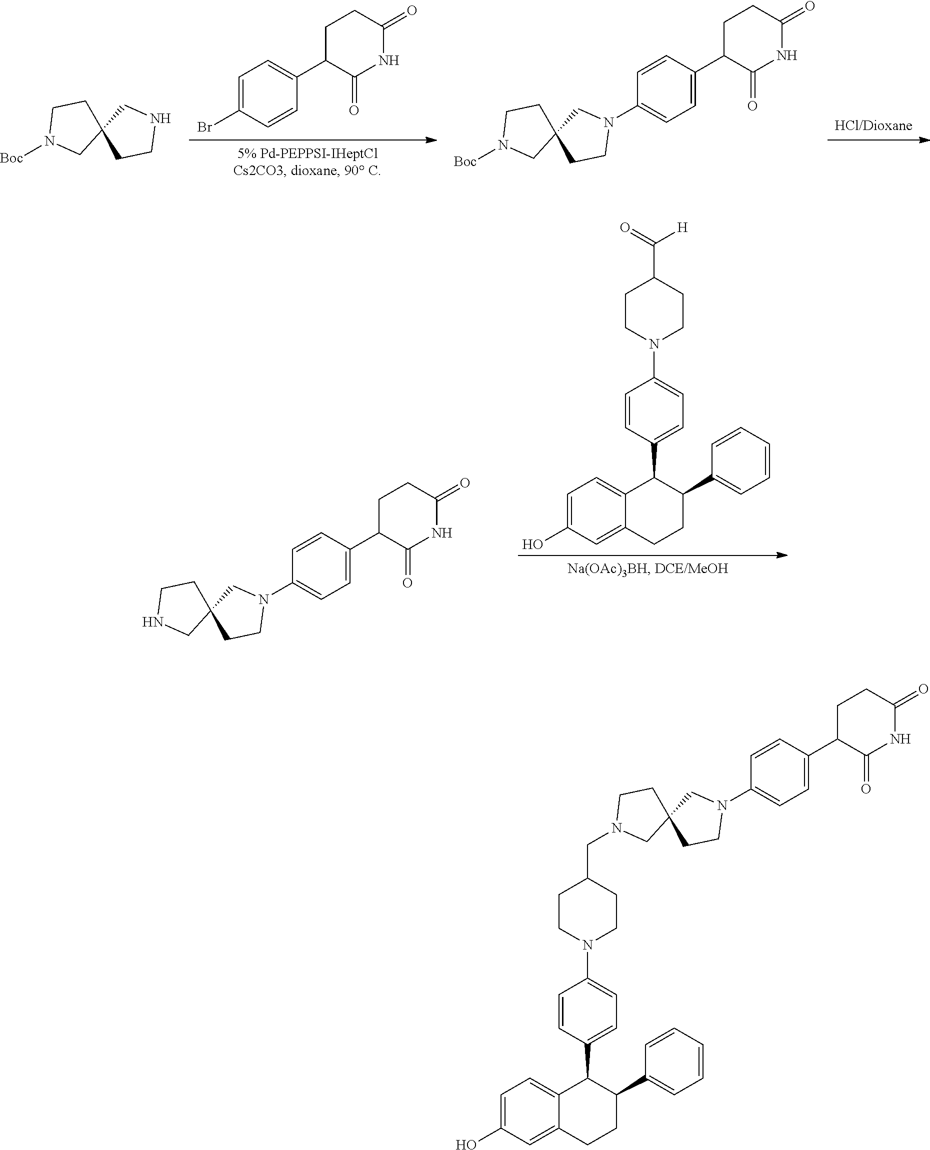 [SMILES: CC(C)(C)OC(=O)N1CC[C@@]2(CCNC2)C1 ; O=C1CCC(c2ccc(Br)cc2)C(=O)N1 ; CC(C)(C)OC(=O)N1CC[C@@]2(CCN(c3ccc(C4CCC(=O)NC4=O)cc3)C2)C1 ; O=C1CCC(c2ccc(N3CC[C@@]4(CCNC4)C3)cc2)C(=O)N1 ; O=CC1CCN(c2ccc([C@@H]3c4ccc(O)cc4CC[C@@H]3c3ccccc3)cc2)CC1 ; O=C1CCC(c2ccc(N3CC[C@@]4(CCN(CC5CCN(c6ccc([C@@H]7c8ccc(O)cc8CC[C@@H]7c7ccccc7)cc6)CC5)C4)C3)cc2)C(=O)N1]

Step 1: To a solution of tert-butyl (R)-2,7-diazaspiro[4.4]nonane-2-carboxylate (300 mg, 1.33 mmol, 2.0 eq), 3-(4-bromophenyl)piperidine-2,6-dione (177.70 mg, 662.79 μmol, 1.0 eq), Cs₂CO₃ (431.90 mg, 1.33 mmol, 2.0 eq) and 5% Pd-PEPPSI-IHeptCI (64.47 mg, 66.28 μmol, 0.1 eq) in dioxane (1.5 mL) was bubbled with N₂ for 1 min. Then the mixture was stirred at 90° C. for 3 hours. LCMS showed the starting material was consumed completely and a major peak with desired MS was detected. The reaction mixture was concentrated to get a residue. The residue was purified by column chromatography (SiO₂, Petroleum ether/Ethyl acetate=1/0 to 0/1) to give tert-butyl (5R)-7-(4-(2,6-dioxopiperidin-3-yl)phenyl)-2,7-diazaspiro[4.4]nonane-2-carboxylate (210 mg, 492.61 μmol, 74.32% yield) as a white solid. LC-MS (ESI⁺) m/z: 414.2 (M+H)⁺. ¹H NMR (400 MHZ, DMSO-d₆) δ ppm 10.74 (s, 1H) 6.95-7.04 (m, 2H) 6.48 (d, J=8.70 Hz, 2H) 3.68 (dd, J=10.61, 4.89 Hz, 1H) 3.35 (br d, J=4.53 Hz, 2H) 3.27-3.31 (m, 2H) 3.13-3.26 (m, 4H) 2.57-2.69 (m, 1H) 2.39-2.48 (m, 1H) 1.78-2.17 (m, 6H) 1.40 (br d, J=6.08 Hz, 9H).

Step 2: A solution of tert-butyl (5R)-7-(4-(2,6-dioxopiperidin-3-yl)phenyl)-2,7-diazaspiro[4.4]nonane-2-carboxylate (100.00 mg, 241.83 μmol, 1.0 eq) in DCM (10 mL) was added dropwise into a solution HCl/Dioxane (4 M, 5 mL, 82.70 eq) at 15° C. for 2 h. LCMS showed that the starting material was consumed and the desired product was detected. The reaction mixture was concentrated to get 3-(4-((R)-2,7-diazaspiro[4.4]nonan-2-yl)phenyl)piperidine-2,6-dione (80 mg, crude) was obtained as a white solid. LC-MS (ESI⁺) m/z: 314.0 (M+H)⁺

Step 3: To a solution of 3-(4-((R)-2,7-diazaspiro[4.4]nonan-2-yl)phenyl)piperidine-2,6-dione (80 mg, 228.67 μmol, 1.0 eq, HCl) in DCE (15 mL) and MeOH (3 mL) was added 1-(4-((1R,2S)-6-hydroxy-2-phenyl-1,2,3,4-tetrahydronaphthalen-1-yl)phenyl)piperidine-4-carbaldehyde (56.46 mg, 137.20 μmol, 0.6 eq) and NaBH(OAc)₃ (242.32 mg, 1.14 mmol, 5.0 eq) at 20° C. Then the mixture was stirred at 20° C. for 2 hours. LCMS showed a major peak with desired MS was detected. The reaction mixture was concentrated to get a residue. The residue was purified by prep-HPLC (column: C18 150×30 mm; mobile phase: [water(NH₄HCO₃)-ACN]; gradient: 85%-100% B over 11 min) to give 3-(4-((R)-7-((1-(4-((1R,2S)-6-hydroxy-2-phenyl-1,2,3,4-tetrahydronaphthalen-1-yl)phenyl)piperidin-4-yl)methyl)-2,7-diazaspiro[4.4]nonan-2-yl)phenyl)piperidine-2,6-dione (16.8 mg, 23.70 μmol, 10.36% yield) as a white solid. LC-MS (ESI⁺) m/z: 709.4 (M+H)⁺.

### Example 153. Preparation of (I-291) 1-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)dihydropyrimidine-2,4 (1H,3H)-dione

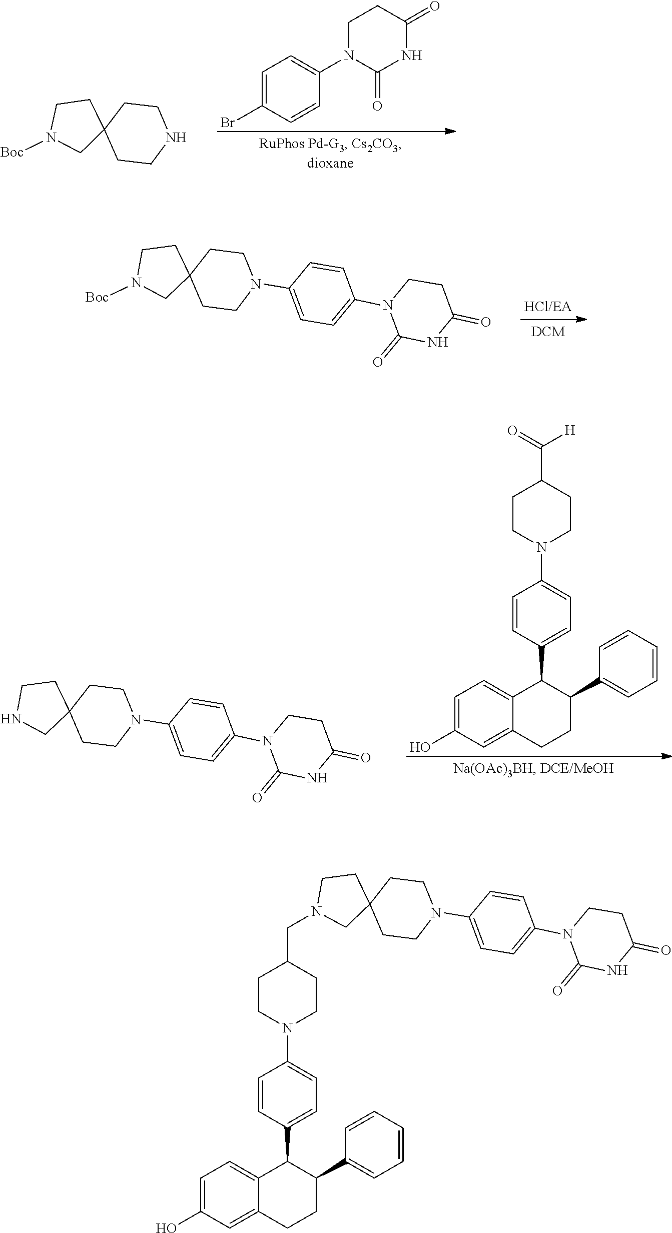 [SMILES: CC(C)(C)OC(=O)N1CCC2(CCNCC2)C1 ; O=C1CCN(c2ccc(Br)cc2)C(=O)N1 ; CC(C)(C)OC(=O)N1CCC2(CCN(c3ccc(N4CCC(=O)NC4=O)cc3)CC2)C1 ; O=C1CCN(c2ccc(N3CCC4(CCNC4)CC3)cc2)C(=O)N1 ; O=CC1CCN(c2ccc([C@@H]3c4ccc(O)cc4CC[C@@H]3c3ccccc3)cc2)CC1 ; O=C1CCN(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1]

Step 1: To a solution of tert-butyl 2,8-diazaspiro[4.5]decane-2-carboxylate (100.03 mg, 416.21 μmol, 2 eq.) in dioxane (1 mL) was added 1-(4-bromophenyl)dihydropyrimidine-2,4 (1H,3H)-dione (56 mg, 208.11 μmol, 1 eq.), Cs₂CO₃ (135.61 mg, 416.21 μmol, 2 eq) and RuPhos Pd G3 (17.41 mg, 20.81 μmol, 0.1 eq). The mixture was stirred at 90° C. under N₂ for 2 hr. LCMS showed the desired ms was found. The mixture was purified by prep-HPLC (Column Boston Green ODS 150*30 mm*5 um; Condition: water (FA)-CAN; Begin B 18, End B 48, Gradient Time (min) 14, 100% B Hold Time (min) 2, FlowRate (ml/min) 25, Injections 6, HPLC 90) to give tort-butyl 8-(4-(2,4-dioxotetrahydropyrimidin-1 (2H)-yl)phenyl)-2,8-diazaspiro[4.5]decane-2-carboxylate (20 mg, 46.67 μmol, 22.43% yield) as a white solid.

Step 2: To a solution of tert-butyl 8-(4-(2,4-dioxotetrahydropyrimidin-1 (2H)-yl)phenyl)-2,8-diazaspiro[4.5]decane-2-carboxylate (20 mg, 46.67 μmol, 1 eq.) in DCM (2 mL) was added HCl/EA (4 M, 116.68 μL, 10 eq.). The reaction was stirred at 20° C. for 1 hr. LCMS showed the desired mass was found. The reaction mixture was concentrated to dryness. The crude product 1-(4-(2,8-diazaspiro[4.5]decan-8-yl)phenyl)dihydropyrimidine-2,4 (1H,3H)-dione (15 mg, 45.67 μmol, 97.86% yield) as a white solid was used into the next step without further purification. LC-MS (ESI⁺) m/z: 329.1 (M+H)⁺.

Step 3: To a solution of 1-(4-(2,8-diazaspiro[4.5]decan-8-yl)phenyl)dihydropyrimidine-2,4 (1H,3H)-dione (15 mg, 45.67 μmol, 1 eq.) in DCE (3 mL) and MeOH (5 mL) was added 1-(4-((1R,2S)-6-hydroxy-2-phenyl-1,2,3,4-tetrahydronaphthalen-1-yl)phenyl)piperidine-4-carbaldehyde (75.19 mg, 182.70 μmol, 4 eq.) and Na(OAc)₃BH (116.16 mg, 548.10 μmol, 12 eq.). The reaction was stirred at 20° C. for 1 hr. LCMS showed the desired ms was found. The mixture was purified by Prep-HPLC (C18-1 150*30 mm*5 um; Condition: water(NH₄HCO₃)-ACN, Begin B 48, End B 78; Gradient Time (min): 11; 100% B Hold Time (min): 2; Flow Rate (ml/min): 25, Injections 1, HPLC 95) to give compound 1-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)dihydropyrimidine-2,4 (1H,3H)-dione (8.2 mg, 11.02 μmol, 24.13% yield, 97.32% purity) as a white solid. LC-MS (ESI⁺) m/z: 724.4 (M+H)⁺.

LCMS: calc. for C₄₆H₅₃N₅O₃:723.96, found: [M+H]⁺724.4.

HPLC: 97.32% purity at 220 nm.

¹H NMR: ¹H NMR (400 MHZ, DMSO-d₆) δ=10.33-10.13 (m, 1H), 9.15-9.03 (m, 1H), 7.16-7.10 (m, 5H), 6.92 (d, J=9.1 Hz, 2H), 6.83 (br d, J=6.7 Hz, 2H), 6.64 (d, J=8.3 Hz, 1H), 6.61-6.59 (m, 1H), 6.52 (d, J=8.7 Hz, 2H), 6.49-6.46 (m, 1H), 6.19 (br d, J=8.6 Hz, 2H), 4.13-4.11 (m, 1H), 3.68 (t, J=6.7 Hz, 2H), 3.52-3.44 (m, 2H), 3.26 (br s, 2H), 3.17-3.03 (m, 4H), 2.99-2.89 (m, 2H), 2.77-2.60 (m, 4H), 2.34-2.32 (m, 2H), 2.27-2.06 (m, 4H), 1.77-1.68 (m, 3H), 1.64-1.55 (m, 6H), 1.27-1.20 (m, 1H), 1.18-1.09 (m, 2H),

### Example 154. Preparation of (I-275) (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione and (I-276) (R)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione

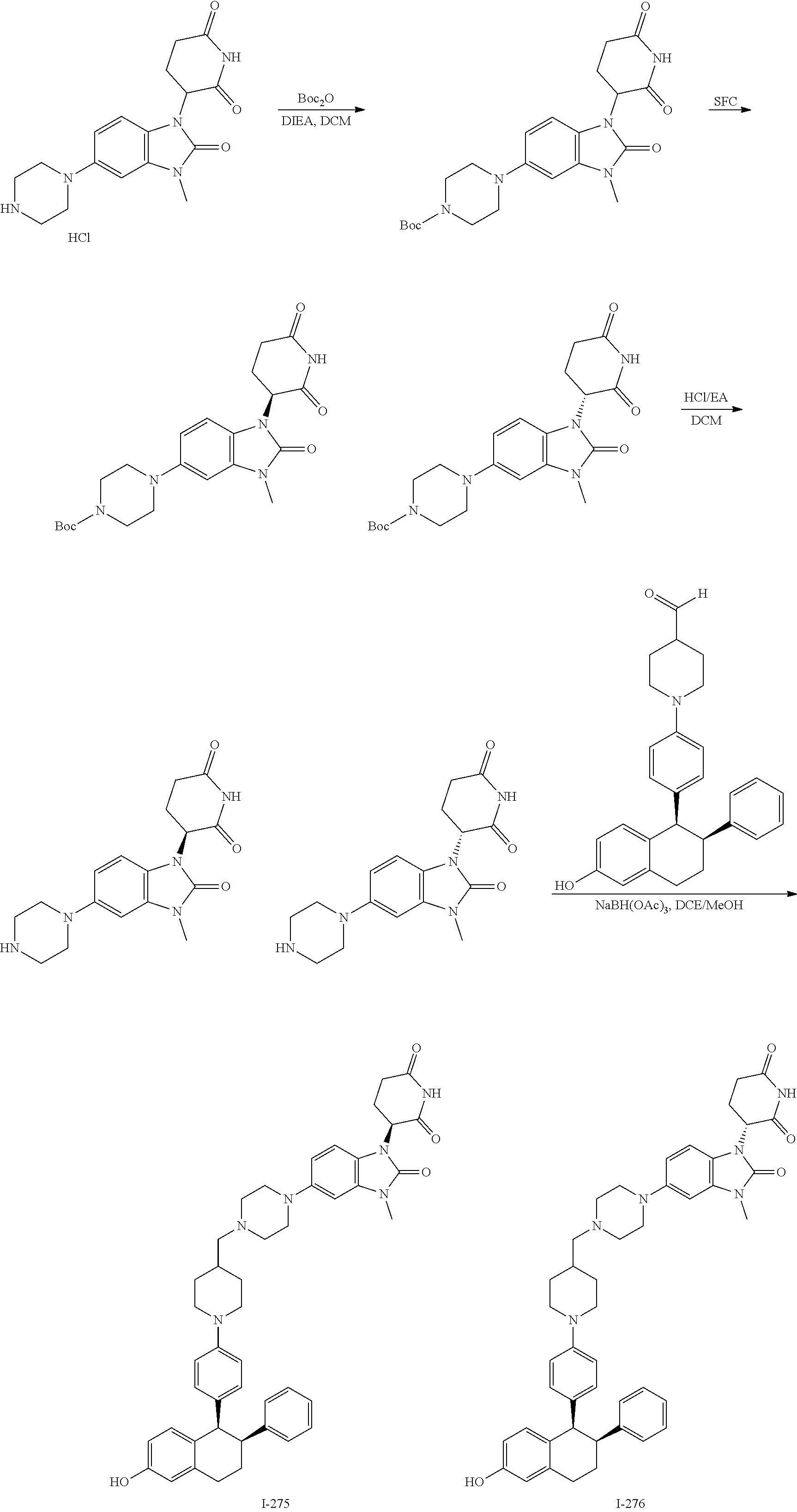

Step 1: To a solution of 3-(3-methyl-2-oxo-5-(piperazin-1-yl)-2,3-dihydro-1H-benzo[d]imidazol-1-yl)piperidine-2,6-dione hydrochloride (1.20 g, 3.16 mmol, 1.0 eq., HCl) in DCM (10 mL) were added Boc₂O (1.09 mL, 4.74 mmol, 1.5 eq.) and DIEA (440.21 μL, 2.53 mmol, 0.8 eq) at 20° C.

The mixture was stirred at 20° C. for 16 hr. LCMS showed the desired MS was detected. The mixture was concentrated to give a residue, which was purified by flash silica gel chromatography eluting with EtOAc in PE from 0% to 100% to give tert-butyl 4-(1-(2,6-dioxopiperidin-3-yl)-3-methyl-2-oxo-2,3-dihydro-1H-benzo[d]imidazol-5-yl)piperazine-1-carboxylate (1.1 g, 87.04% purity) as a white solid. LC-MS (ESI⁺) m/z: 444.3 (M+H+).

Step 2: tert-butyl 4-(1-(2,6-dioxopiperidin-3-yl)-3-methyl-2-oxo-2,3-dihydro-1H-benzo[d]imidazol-5-yl)piperazine-1-carboxylate (1.1 g, 2.48 mmol, 1 eq.) was separated by chiral SFC (column: DAICEL CHIRALPAK IG 250 mm § 30 mm, 10 um); mobile phase: [CO₂-EtOH (0.1% NH₃H₂O)]; B %: 60%-60%; Flow Rate: 80 ml/min; peak 1:2.643 min; peak 2:3.950 min) to yield peak 1 and peak 2.

Peak 1 was concentrated under reduced pressure to yield tert-butyl (S)-4-(1-(2,6-dioxopiperidin-3-yl)-3-methyl-2-oxo-2,3-dihydro-1H-benzo[d]imidazol-5-yl)piperazine-1-carboxylate (265.2 mg, 24.11% yield) as a white solid. LC-MS (ESI⁺) m/z: 444.2 (M+H)⁺.

Peak 2 was concentrated under reduced pressure to yield tert-butyl (R)-4-(1-(2,6-dioxopiperidin-3-yl)-3-methyl-2-oxo-2,3-dihydro-1H-benzo[d]imidazol-5-yl)piperazine-1-carboxylate (251.3 mg, 22.85% yield) as a white solid. LC-MS (ESI⁺) m/z: 444.2 (M+H)⁺.

Step 3: To a solution of ter-butyl (S)-4-(1-(2,6-dioxopiperidin-3-yl)-3-methyl-2-oxo-2,3-dihydro-1H-benzo[d]imidazol-5-yl)piperazine-1-carboxylate (80.00 mg, 180.39 μmol, 1 eq.) in DCM (10 mL) was added HCl/EtOAc (450.96 μL, 1.80 mmol, 208.51 eq.). The mixture was stirred at 20° C. for 1 hr. LCMS showed the desired ms was found. After completion, the mixture was concentrated under reduced pressure to give (S)-3-(3-methyl-2-oxo-5-(piperazin-1-yl)-2,3-dihydro-1H-benzo[d]imidazol-1-yl)piperidine-2,6-dione (50.00 mg, crude) as a yellow solid without purification. LC-MS (ESI⁺) m/z: 344.0 (M+H)⁺

Step 4: To a solution of tert-butyl (R)-4-(1-(2,6-dioxopiperidin-3-yl)-3-methyl-2-oxo-2,3-dihydro-1H-benzo[d]imidazol-5-yl)piperazine-1-carboxylate (80.00 mg, 172.18 μmol, 1 eq.) in DCM (10 mL) was added HCl/EtOAc (450.96 μL, 1.80 mmol, 208.51 eq.). The mixture was stirred at 20° C. for 1 hr. LCMS showed the desired ms was found. After completion, the mixture was concentrated under reduced pressure to give (R)-3-(3-methyl-2-oxo-5-(piperazin-1-yl)-2,3-dihydro-1H-benzo[d]imidazol-1-yl)piperidine-2,6-dione (50.00 mg, crude) as a yellow solid without purification. LC-MS (ESI⁺) m/z: 344.0 (M+H)⁺.

Step 5: To a solution of (S)-3-(3-methyl-2-oxo-5-(piperazin-1-yl)-2,3-dihydro-1H-benzo[d]imidazol-1-yl)piperidine-2,6-dione (50 mg, 145.61 μmol, 1 eq.) in DCE (5 mL) and MeOH (7 mL) was added 1-(4-((1R,2S)-6-hydroxy-2-phenyl-1,2,3,4-tetrahydronaphthalen-1-yl)phenyl)piperidine-4-carbaldehyde (239.70 mg, 582.45 μmol, 4 eq.) and NaBH(OAc)₃ (370.33 mg, 1.75 mmol, 12 eq.). The mixture was stirred at 20° C. for 1 hr. The mixture was purified by Prep-HPLC (Column: C18-1 150*30 mm*5 um; Condition water(NH₄HCO₃)-CAN, B %: 48%-78%, 11 min) to give (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione (24.3 mg, 32.31 μmol, 22.19% yield, 98.25% purity) as a white solid. LC-MS (ESI⁺) m/z: 739.4 (M+H)⁺.

LCMS: calc. for C₄₅H₅₆N₆O₄:738.93, found: [M+H]⁺739.4.

HPLC: 98.25% purity at 220 nm.

¹H NMR: 1H NMR (400 MHZ, DMSO-d₆) δ ppm 11.24-10.90 (m, 1H), 9.10 (s, 1H), 7.17-7.09 (m, 5H), 6.93 (d, J=8.7 Hz, 1H), 6.84-6.83 (m, 2H), 6.65-6.63 (m, 1H), 6.60 (br s, 1H), 6.54 (s, 1H), 6.52 (s, 1H), 6.48 (d, J=5.8 Hz, 1H), 6.20 (br d, J=8.7 Hz, 2H), 5.31-5.26 (m, 1H), 4.12 (br d, J=4.6 Hz, 1H), 3.53-3.49 (m, 2H), 3.30 (d, J=2.7 Hz, 6H), 3.07 (br s, 3H), 2.97-2.87 (m, 4H), 2.73-2.66 (m, 2H), 2.19 (br d, J=7.2 Hz, 2H), 2.12-2.07 (m, 1H), 2.00-1.96 (m, 1H), 1.77-1.61 (m, 6H), 1.28-1.11 (m, 4H).

Step 6: To a solution of (R)-3-(3-methyl-2-oxo-5-(piperazin-1-yl)-2,3-dihydro-1H-benzo[d]imidazol-1-yl)piperidine-2,6-dione (50 mg, 145.61 μmol, 1 eq.) in DCE (5 mL) and MeOH (7 mL) was added 1-(4-((1R,2S)-6-hydroxy-2-phenyl-1,2,3,4-tetrahydronaphthalen-1-yl)phenyl)piperidine-4-carbaldehyde (239.70 mg, 582.45 μmol, 4 eq.) and NaBH(OAc)₃ (370.33 mg, 1.75 mmol, 12 eq.). The mixture was stirred at 20° C. for 1 hr. The mixture was purified by Prep-HPLC (Column: C18-1 150*30 mm*5 um; Condition water(NH₄HCO₃)-CAN, B %: 48%-78%, 11 min) to give (R)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione (34.1 mg, 45.52 μmol, 31.26% yield, 98.63% purity) as a white solid. LC-MS (ESI⁺) m/z: 739.4 (M+H)⁺.

LCMS: calc. for C₄₅H₅₀N₆O₄: 738.93, found: [M+H]⁺739.4.

HPLC: 98.63% purity at 220 nm.

¹H NMR: 1H NMR (400 MHZ, DMSO-d₆) δ ppm 11.15-10.99 (m, 1H), 9.10 (s, 1H), 7.17-7.09 (m, 5H), 6.93 (d, J=8.7 Hz, 1H), 6.84-6.83 (m, 2H), 6.65-6.63 (m, 1H), 6.60 (br s, 1H), 6.54 (s, 1H), 6.52 (s, 1H), 6.48 (d, J=6.0 Hz, 1H), 6.20 (d, J=8.6 Hz, 2H), 5.31-5.26 (m, 1H), 4.12 (br d, J=4.9 Hz, 1H), 3.53-3.49 (m, 2H), 3.30 (d, J=2.6 Hz, 6H), 3.08 (br s, 3H), 2.98-2.88 (m, 4H), 2.74-2.66 (m, 2H), 2.19 (br d, J=7.0 Hz, 2H), 2.12-2.07 (m, 1H), 1.99-1.96 (m, 1H), 1.76-1.60 (m, 6H), 1.24-1.09 (m, 4H).

EXAMPLE 155, Preparation of (I-295) (R)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione and (I-296) (S)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione

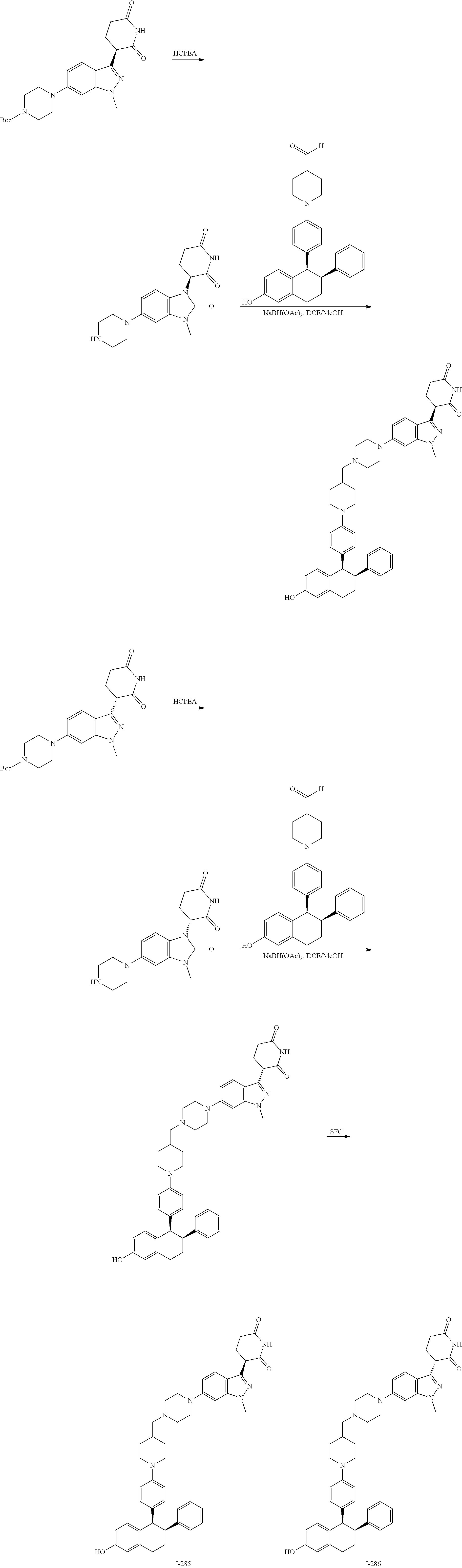

Step 1: A solution of tert-butyl (R)-4-(3-(2,6-dioxopiperidin-3-yl)-1-methyl-1H-indazol-6-yl)piperazine-1-carboxylate (63 mg, 147.37 μmol, 1 eq.) in DCM (10 mL) was added dropwise into a solution HCl/Dioxane (4 M, 4 mL) at 15° C. for 2 h. LCMS showed that the starting material was consumed and the desired product was detected. The reaction mixture was concentrated to get (R)-3-(1-methyl-6-(piperazin-1-yl)-1H-indazol-3-yl)piperidine-2,6-dione (50 mg, crude) was obtained as a white solid. LC-MS (ESI⁺) m/z: 328.2 (M+H)⁺.

Step 2: To a solution of (R)-3-(1-methyl-6-(piperazin-1-yl)-1H-indazol-3-yl)piperidine-2,6-dione (55.00 mg, 151.16 μmol, 1.0 eq., HCl) in DCE (5 mL) and MeOH (5 mL) was added 1-(4-((1R,2S)-6-hydroxy-2-phenyl-1,2,3,4-tetrahydronaphthalen-1-yl)phenyl)piperidine-4-carbaldehyde (93.31 mg, 226.75 μmol, 1.5 eq.) at 20° C. After 5 minutes, the NaBH(OAc)₃ (192.23 mg, 906.99 μmol, 6.0 eq.) was added into the mixture at 20° C. The mixture was stirred at 20° C. for 0.5 h. LCMS showed that the desired product was detected. The mixture was filtered and concentrated under vacuum to give the crude. The residue was purified by prep-HPLC (condition: Column: Boston Green ODS 150*30 mm*5 um; Condition: water (FA)-ACN, Begin B 55, End B 75, Gradient Time (min) 12, 100% B Hold Time (min) 2, Flow Rate (mL/min) 25.) to give (R)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione (28 mg, 24.67% yield) as white solid.

LC-MS (ESI⁺) m/z: 723.4 (M+H)⁺.

¹H NMR (400 MHZ, DMSO-d₆) δ=10.85 (s, 1H), 9.10 (s, 1H), 8.13 (s, 1H), 7.51 (br d, J=7.2 Hz, 1H), 7.18-7.10 (m, 3H), 6.97-6.81 (m, 4H), 6.65-6.47 (m, 5H), 6.21 (br d, J=8.3 Hz, 2H), 4.30-4.22 (m, 1H), 4.13 (br d, J=4.8 Hz, 1H), 3.89 (s, 3H), 3.56-3.49 (m, 2H), 3.32 (s, 8H), 3.20 (br d, J=3.3 Hz, 2H), 3.02-2.91 (m, 2H), 2.69-2.56 (m, 4H), 2.34-2.11 (m, 4H), 1.84-1.60 (m, 4H), 1.27-1.11 (m, 2H).

Step 3: A solution of tert-butyl (S)-4-(3-(2,6-dioxopiperidin-3-yl)-1-methyl-1H-indazol-6-yl)piperazine-1-carboxylate (69 mg, 161.41 μmol, 1 eq.) in DCM (10 mL) was added dropwise into a solution HCl/Dioxane (4 M, 1.20 mL) at 15° C. for 2 h. LCMS showed that the starting material was consumed and the desired product was detected. The reaction mixture was concentrated to get (S)-3-(1-methyl-6-(piperazin-1-yl)-1H-indazol-3-yl)piperidine-2,6-dione (55 mg, crude) as a white solid. LC-MS (ESI⁺) m/z: 328.2 (M+H)⁺.

Step 4: To a solution of (S)-3-(1-methyl-6-(piperazin-1-yl)-1H-indazol-3-yl)piperidine-2,6-dione (55 mg, 151.16 μmol, 1.0 eq., HCl) in DCE (5 mL) and MeOH (5 mL) was added 1-(4-((1R,2S)-6-hydroxy-2-phenyl-1,2,3,4-tetrahydronaphthalen-1-yl)phenyl)piperidine-4-carbaldehyde (93.31 mg, 226.75 μmol, 1.5 eq.) at 20° C. After 5 minutes, the NaBH(OAc) (192.23 mg, 906.99 μmol, 6.0 eq.) was added into the mixture at 20° C. The mixture was stirred at 20° C. for 12 h. LCMS showed that the desired product was detected. The mixture was filtered and concentrated under vacuum to give the crude. The residue was purified by prep-HPLC (condition: Column: Boston Green ODS 150*30 mm*5 um; Condition: water (FA)-ACN, Begin B 55, End B 75, Gradient Time (min) 12, 100% B Hold Time (min) 2, Flow Rate (mL/min) 25.) to give (S)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione (25 mg, 22.57% yield) as white solid.

LC-MS (ESI⁺) m/z: 723.4 (M+H)⁺.

¹H NMR (400 MHZ, DMSO-d₆) δ=10.85 (s, 1H), 9.10 (s, 1H), 8.13 (s, 1H), 7.51 (br d, J=9.1 Hz, 1H), 7.18-7.09 (m, 3H), 6.95-6.80 (m, 4H), 6.66-6.46 (m, 5H), 6.20 (d, J=8.6 Hz, 2H), 4.32-4.19 (m, 1H), 4.13 (d, J=4.9 Hz, 1H), 3.89 (s, 3H), 3.56-3.49 (m, 2H), 3.32 (s, 8H), 3.24-3.16 (m, 2H), 2.99-2.88 (m, 2H), 2.69-2.57 (m, 4H), 2.33-2.11 (m, 4H), 1.82-1.63 (m, 4H), 1.18 (br d, J=9.1 Hz, 2H).

Step 5: The (R)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione and (S)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione (53 mg, 73.31 μmol, 1.0 eq.) was further separated by SFC (condition: CO₂-EtOH, column: DAICEL CHIRALPAK ID (250 mm*30 mm*10 um). to give (R)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione (13.0 mg, 24.53% yield) as white solid and (S)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione (13.0 mg, 24.53% yield) to give solid.

(R)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione:

LCMS: calc. for C₄₅H₅₀N₆O₃: 722.93, found: [M+H]⁺723.4.

HPLC: 100.00% purity at 220 nm

¹H NMR (400 MHz, DMSO-d₆) δ=10.85 (s, 1H), 9.10 (s, 1H), 7.49 (d, J=8.9 Hz, 1H), 7.17-7.08 (m, 3H), 6.93-6.81 (m, 4H), 6.66-6.47 (m, 5H), 6.20 (d, J=8.6 Hz, 2H), 4.31-4.20 (m, 1H), 4.13 (d, J=4.6 Hz, 1H), 3.88 (s, 3H), 3.56-3.47 (m, 2H), 3.32 (s, 8H), 3.21 (br s, 2H), 3.02-2.85 (m, 2H), 2.69-2.54 (m, 3H), 2.33-2.13 (m, 5H), 1.79-1.62 (m, 4H), 1.22-1.12 (m, 2H).

(S)-3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione:

LCMS: calc. for C₄₅H₅₀N₆O₃: 722.93, found: [M+H]⁺723.4.

HPLC: 100.00% purity at 220 nm

¹H NMR (400 MHz, DMSO-d₆) δ=10.85 (s, 1H), 9.10 (s, 1H), 7.49 (d, J=9.1 Hz, 1H), 7.18-7.09 (m, 3H), 6.93-6.80 (m, 4H), 6.66-6.46 (m, 5H), 6.20 (d, J=8.6 Hz, 2H), 4.33-4.21 (m, 1H), 4.13 (d, J=4.6 Hz, 1H), 3.88 (s, 3H), 3.57-3.46 (m, 2H), 3.32 (s, 8H), 3.21 (br s, 2H), 3.01-2.86 (m, 2H), 2.70-2.56 (m, 3H), 2.33-2.10 (m, 5H), 1.80-1.60 (m, 4H), 1.23-1.12 (m, 2H).

### Example 156. Preparation of (I-189) 3-(7-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione

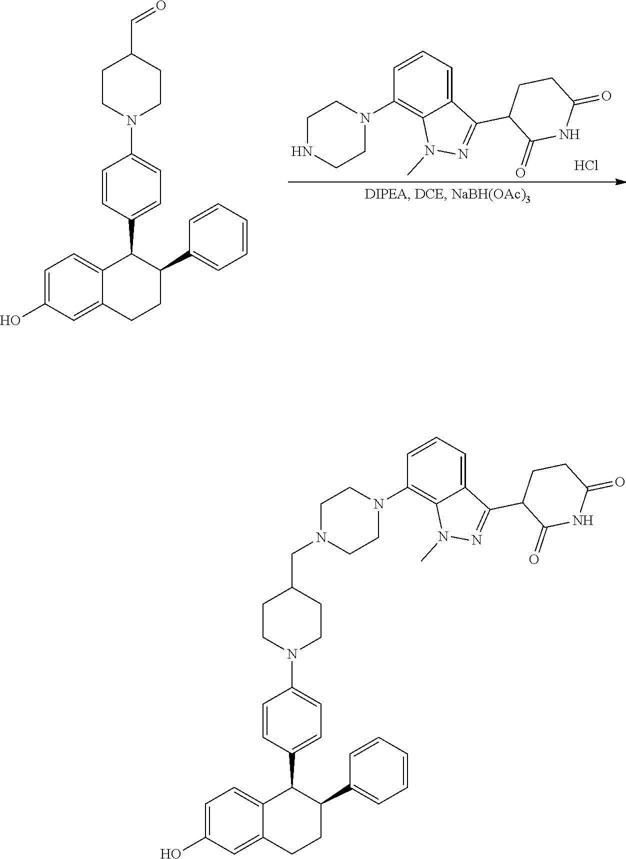 [SMILES: O=CC1CCN(c2ccc([C@@H]3c4ccc(O)cc4CC[C@@H]3c3ccccc3)cc2)CC1 ; Cn1nc(C2CCC(=O)NC2=O)c2cccc(N3CCNCC3)c21 ; Cl ; Cn1nc(C2CCC(=O)NC2=O)c2cccc(N3CCN(CC4CCN(c5ccc([C@@H]6c7ccc(O)cc7CC[C@@H]6c6ccccc6)cc5)CC4)CC3)c21]

Step 1: To a solution of 1-(4-((1R,2S)-6-hydroxy-2-phenyl-1,2,3,4-tetrahydronaphthalen-1-yl)phenyl)piperidine-4-carbaldehyde (90 mg, 1 Eq, 0.22 mmol) in DCE (10 mL) was added 3-(1-methyl-7-(piperazin-1-yl)-1H-indazol-3-yl)piperidine-2,6-dione hydrochloride (95 mg, 1.2 Eq, 0.26 mmol) and NaBH(OAc)₃ (93 mg, 2 Eq, 0.44 mmol). The mixture was adjusted to pH 5-6 with DIEA (28 mg, 38 μL, 1 Eq, 0.22 mmol). The mixture was stirred at 25° C. for 3 hours. LCMS showed the reaction was completed. The mixture was concentrated and then water (30 mL) was added. The mixture was extracted with DCM (20 mL×3). The combined organic layers were dried over Na₂SO₄, filtered and concentrated. The residue was purified by HPLC (Column Boston Green ODS 150*30 mm*5 um; Condition water (FA)-ACN; Begin B 20; End B 50; Gradient Time (min) 12; 100% B Hold Time (min) 2; FlowRate (ml/min) 25) to give 3-(7-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dion (20 mg, 28 μmol, 13%) as an off-white solid. LC-MS (ESI⁺) m/z: 723.4 (M+H)⁺.

LCMS: calc. for C₄₅H₅₀N₆O₄: 722.93, found: [M+H]⁺723.4.

HPLC: 99.78% purity at 220 nm.

¹H NMR (400 MHZ, METHANOL-d₄) δ ppm: 8.41-8.48 (m, 1H), 7.44 (d, J=7.51 Hz, 1H), 7.06-7.14 (m, 5H), 6.78-6.83 (m, 2H), 6.61-6.71 (m, 4H), 6.52 (dd, J=8.40, 2.32 Hz, 1H), 6.31 (d, J=8.58 Hz, 2H), 4.30-4.37 (m, 4H), 4.19 (d, J=4.53 Hz, 1H), 3.55 (br dd, J=9.66, 2.26 Hz, 2H), 3.12-3.26 (m, 4H), 2.96-3.09 (m, 4H), 2.72-2.80 (m, 2H), 2.62-2.69 (m, 2H), 2.56 (br d, J=11.92 Hz, 3H), 2.44 (td, J=9.15, 4.23 Hz, 1H), 2.20-2.34 (m, 2H), 1.86-1.93 (m, 2H), 1.74-1.83 (m, 2H), 1.25-1.45 (m, 4H).

### Example 157. Preparation of (I-188) 3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione

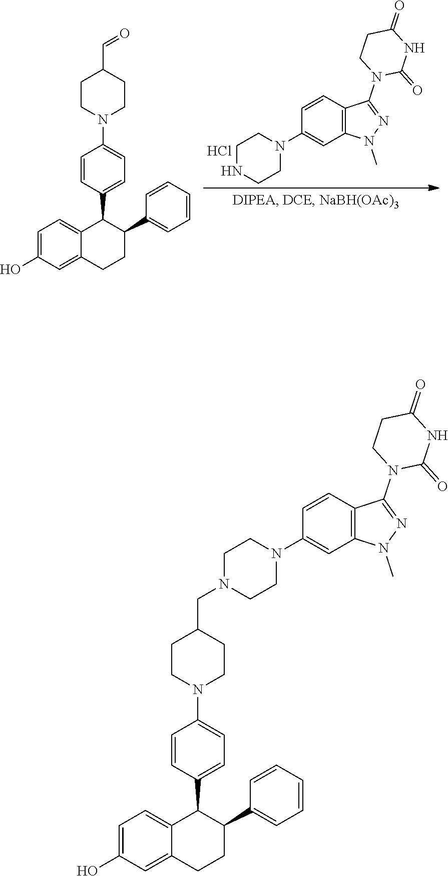 [SMILES: O=CC1CCN(c2ccc([C@@H]3c4ccc(O)cc4CC[C@@H]3c3ccccc3)cc2)CC1 ; Cl ; Cn1nc(N2CCC(=O)NC2=O)c2ccc(N3CCNCC3)cc21 ; Cn1nc(N2CCC(=O)NC2=O)c2ccc(N3CCN(CC4CCN(c5ccc([C@@H]6c7ccc(O)cc7CC[C@@H]6c6ccccc6)cc5)CC4)CC3)cc21]

Step 1: To a solution of 1-(4-((1R,2S)-6-hydroxy-2-phenyl-1,2,3,4-tetrahydronaphthalen-1-yl)phenyl)piperidine-4-carbaldehyde (50 mg, 0.12 mmol, 1.0 eq.) and 3-(1-methyl-6-(piperazin-1-yl)-1H-indazol-3-yl)piperidine-2,6-dione (48 mg, 1.2 eq., 0.15 mmol) in DCE (3 mL) and MeOH (3 mL) was added Sodium triacetoxyborohydride (77 mg, 0.36 mmol, 3.0 eq.). The mixture was stirred at 20° C. for 2 hour. LCMS showed the reaction was completed. The crude was purified by prep-HPLC together (Welch Xtimate C18 150*25 mm*5 um, water (FA)-ACN as a mobile phase, from 20% to 55%, Gradient Time (min): 11, Flow Rate (ml/min): 25) to give 3-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)piperidine-2,6-dione (15.5 mg, 21.4 μmol, 18%) as a white solid. LC-MS (ESI⁺) m/z: 723.6 (M+H)⁺.

LCMS: calc. for C₄₅H₅₀N₆O₃, 722.93 found: [M+H]⁺723.6 HPLC: 100.00% purity at 220 nm.

¹H NMR (400 MHZ, METHANOL-d₄) δ: ppm 8.21-8.68 (m, 1H), 7.56 (d, J=9.20 Hz, 1H), 7.08-7.13 (m, 3H), 6.94-7.01 (m, 1H), 6.78-6.85 (m, 3H), 6.69 (d, J=8.40 Hz, 1H), 6.63 (d, J=8.40 Hz, 3H), 6.52 (m, 1H), 6.31 (d, J=8.40 Hz, 2H), 4.29 (m, 1H), 4.18 (d, J=5.20 Hz, 1H), 3.95 (s, 3H), 3.50-3.57 (m, 2H), 3.36 (s, 4H), 2.96-3.05 (m, 2H), 2.79-2.91 (m, 4H), 2.69-2.79 (m, 2H), 2.60 (t, J=11.20 Hz, 2H), 2.49-2.56 (m, 2H), 2.43 (m, 1H), 2.19-2.32 (m, 2H), 1.88 (d, J=13.60 Hz, 2H), 1.72-1.82 (m, 2H), 1.27-1.46 (m, 3H).

### Example 158. Preparation of (I-274) 3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl) pyrrolidine-2,5-dione

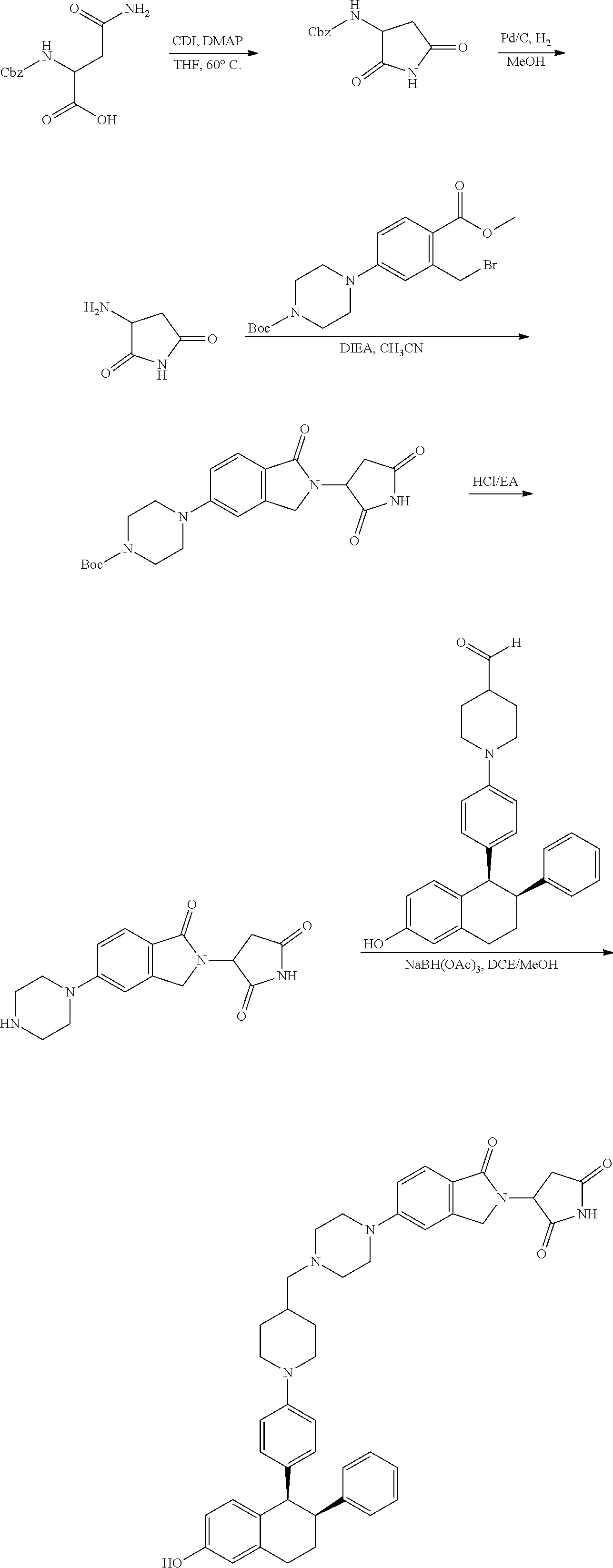

Step 1: To a solution of ((benzyloxy) carbonyl) asparagine (1.5 g, 5.63 mmol, 1 eq.) in THF (20 mL) was added DMAP (137.66 mg, 1.13 mmol, 0.2 eq.) and CDI (913.52 mg, 5.63 mmol, 1 eq.). The mixture was stirred at 60° C. for 10 hr. LC-MS showed one main peak with desired mass was detected. The reaction mixture was partitioned between EA (50 mL×2) and water (50 mL). After quenching the reaction, the reaction mixture was poured into separatory funnel and separated. The residue was purified by prep-HPLC (Column Boston Green ODS 150*30 mm*5 um; Condition: water (FA)-CAN; Begin B 22, End B 32, Gradient Time (min) 12, 100% B Hold Time (min) 2, FlowRate (ml/min) 25, Injections 4, HPLC 90). Compound benzyl N-(2,5-dioxopyrrolidin-3-yl) carbamate (1 g, 4.03 mmol, 71.50% yield) was obtained as a brown liquid. LC-MS (ESI⁺) m/z: 249.3 (M+H)⁺.

Step 2: The mixture of benzyl N-(2,5-dioxopyrrolidin-3-yl) carbamate (1 g, 4.03 mmol, 1 eq.), Pd/C (8.57 g, 8.06 mmol, 10% purity, 2 eq.) in MeOH (10 mL) was degassed and the mixture was degassed and refilled with H₂ twice. The reaction mixture was stirred at 25° C. on 15 psi with H₂ for 24 hr. LC-MS showed one main peak with desired mass was detected. The reaction mixture or solution was concentrated to dryness. The crude product 3-aminopyrrolidine-2,5-dione (300 mg, 2.63 mmol, 65.27% yield) as yellow liquid was used into the next step without further purification. LC-MS (ESI⁺) m/z: 115.2 (M+H)⁺.

Step 3: To a solution of 3-aminopyrrolidine-2,5-dione (50 mg, 438.20 μmol, 1 eq.) and tert-butyl 4-(3-(bromomethyl)-4-(methoxycarbonyl)phenyl)piperazine-1-carboxylate (181.11 mg, 438.20 μmol, 1 eq.) in CH₃CN (3 mL) was added DIPEA (283.17 mg, 2.19 mmol, 381.63 μL, 5 eq.). The mixture was stirred at 80° C. for 3 hr. LC-MS showed one peak with desired mass was detected. The reaction mixture or solution was concentrated to dryness. The residue was purified by prep-HPLC (Column Boston Green ODS 150*30 mm*5 um; Condition water (FA)-CAN; Begin B 33, End B 63, Gradient Time (min) 12, 100% B Hold Time (min) 2, FlowRate (ml/min) 25, Injections 6, HPLC 90). tert-butyl 4-(2-(2,5-dioxopyrrolidin-3-yl)-1-oxoisoindolin-5-yl)piperazine-1-carboxylate (12 mg, 28.95 μmol, 6.61% yield) was obtained as a white solid. LC-MS (ESI⁺) m/z: 415.3 (M+H)⁺.

Step 4: To a solution of tert-butyl 4-(2-(2,5-dioxopyrrolidin-3-yl)-1-oxoisoindolin-5-yl)piperazine-1-carboxylate (12 mg, 28.95 μmol, 1 eq) in DCM (5 mL) was added HCl/EA (72.38 μL, 289.54 μmol, 10 eq). The reaction was stirred at 20° C. for 1 hr. LC-MS showed the desired mass was found. The mixture was used for the next step directly. The crude product is about 9 mg. LC-MS (ESI⁺) m/z: 315.2 (M+H)⁺.

Step 5: To a solution of 3-(1-oxo-5-(piperazin-1-yl)isoindolin-2-yl) pyrrolidine-2,5-dione (9 mg, 28.63 μmol, 1 eq) in DCM (3 mL) and MeOH (5 mL) was added 1-(4-((1R,2S)-6-hydroxy-2-phenyl-1,2,3,4-tetrahydronaphthalen-1-yl)phenyl)piperidine-4-carbaldehyde (47.13 mg, 114.53 μmol, 4 eq) and Na(OAc)₃BH (72.82 mg, 343.58 μmol, 12 eq). The reaction was stirred at 20° C. for 6 hr. LC-MS showed the desired ms was found. The mixture was purified by Prep-HPLC (Boston Green ODS 150*30 mm*5 um; Condition: water (HCl)-ACN, Begin B 20, End B 40; Gradient Time (min): 10.5; 100% B Hold Time (min): 1.5; Flow Rate (ml/min): 25) to give 3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl) pyrrolidine-2,5-dione (4 mg, 5.63 μmol, 19.68% yield, 100% purity) as a white solid. LC-MS (ESI⁺) m/z: 710.3 (M+H)⁺.

LCMS: calc. for C₄₄H₄₇N₅O₄:709.89, found: [M+H]⁺710.3.

HPLC: 100% purity at 220 nm.

¹H NMR: ¹H NMR (400 MHZ, DMSO-d₆) δ=11.54-11.47 (m, 1H), 9.32-9.05 (m, 1H), 7.58 (d, J=8.3 Hz, 1H), 7.20-7.10 (m, 6H), 6.84 (br d, J=6.6 Hz, 2H), 6.72-6.59 (m, 3H), 6.55-6.42 (m, 2H), 6.40-6.08 (m, 2H), 5.19 (dd, J=6.7, 8.9 Hz, 1H), 4.53 (d, J=17.2 Hz, 1H), 4.27 (d, J=17.3 Hz, 1H), 4.00 (br d, J=13.4 Hz, 2H), 3.64-3.52 (m, 6H), 3.37-3.25 (m, 4H), 3.18-3.09 (m, 4H), 3.01-2.89 (m, 4H), 2.14-1.89 (m, 5H), 1.79-1.66 (m, 2H),

### Example 159. Preparation of (I-340) (3S)-3-[5-[4-[[1-[4-[(1R,2S)-6-hydroxy-2-(2,3,4,5,6-pentadeuteriophenyl)tetralin-1-yl]phenyl]-4-piperidyl]methyl]piperazin-1-yl]-1-oxo-isoindolin-2-yl]piperidine-2,6-dione

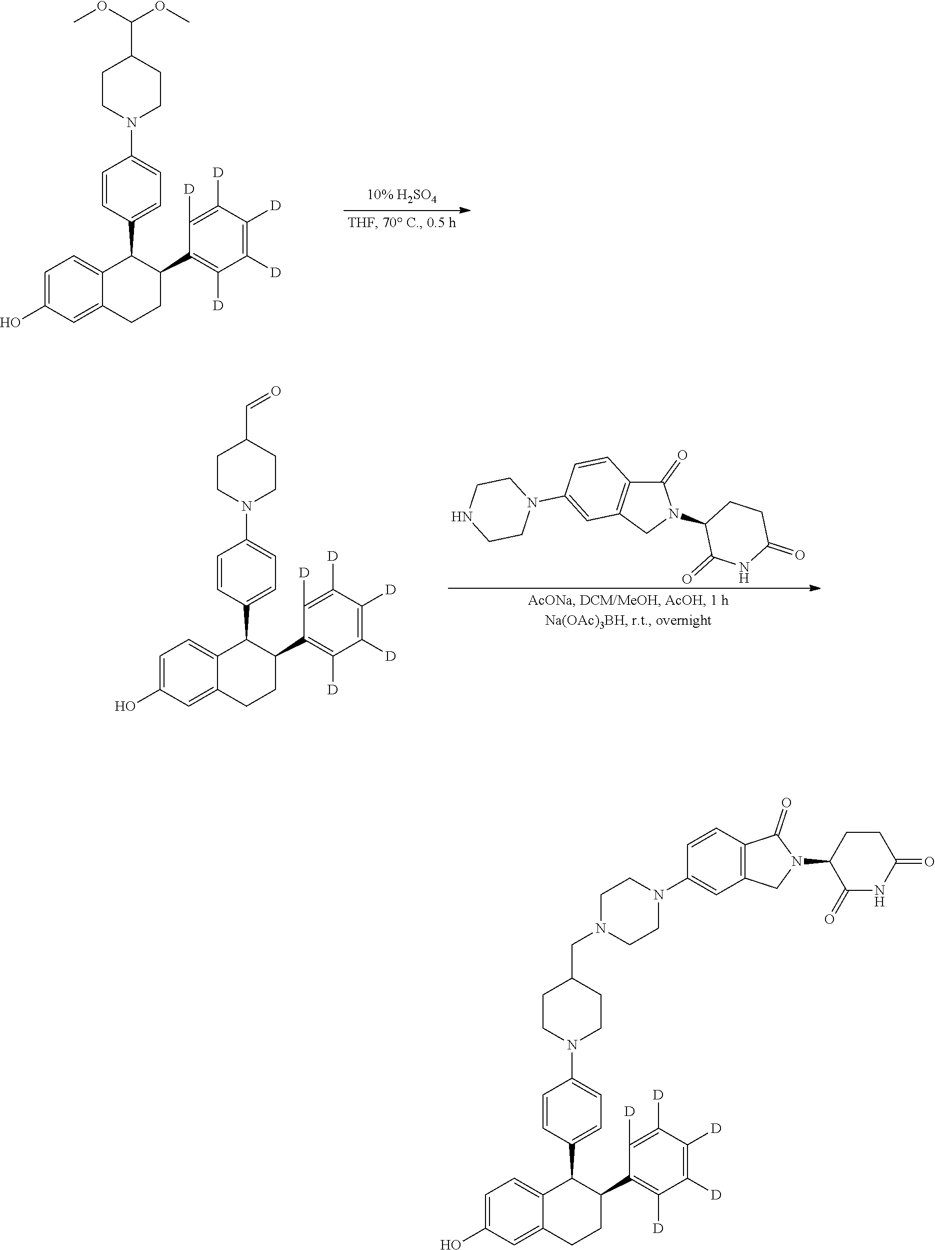 [SMILES: [2H]c1c([2H])c([2H])c([C@H]2CCc3cc(O)ccc3[C@H]2c2ccc(N3CCC(C(OC)OC)CC3)cc2)c([2H])c1[2H] ; [2H]c1c([2H])c([2H])c([C@H]2CCc3cc(O)ccc3[C@H]2c2ccc(N3CCC(C=O)CC3)cc2)c([2H])c1[2H] ; O=C1CC[C@H](N2Cc3cc(N4CCNCC4)ccc3C2=O)C(=O)N1 ; [2H]c1c([2H])c([2H])c([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)c([2H])c1[2H]]

Step 1: To a solution of (1R,2S)-1-[4-[4-(dimethoxymethyl)-1-piperidyl]phenyl]-2-(2,3,4,5,6-pentadeuteriophenyl)tetralin-6-ol (100 mg, 216.15 μmol, 1 eq) in 10% H₂SO₄ (2 mL) and THF (2 mL). The mixture was stirred at 70° C. for 2 hr. The mixture was alkalied with NaHCO₃ to pH=7-8. The reaction mixture was diluted with 50 mL H₂O and extracted with ethyl acetate (50 mL*2). The combined organic layers were washed with 50 mL brine, dried over Na₂SO₄, filtered and concentrated under reduced pressure to give a residue. The crude product 1-[4-[(1R,2S)-6-hydroxy-2-(2,3,4,5,6-pentadeuteriophenyl)tetralin-1-yl]phenyl]piperidine-4-carbaldehyde (87 mg, 208.85 μmol, 96.62% yield) as yellow oil was used into the next step without further purification.

LC-MS (ESI⁺) m/z: 417.3 (M+H)⁺

Step 2: A mixture of 1-[4-[(1R,2S)-6-hydroxy-2-(2,3,4,5,6-pentadeuteriophenyl)tetralin-1-yl]phenyl]piperidine-4-carbaldehyde (87 mg, 208.9 μmol, 1 eq), (S)-3-(1-oxo-5-(piperazin-1-yl)isoindolin-2-yl)piperidine-2,6-dione benzenesulfonate (152.4 mg, 313.3 μmol, 1.5 eq) and sodium acetate (51.4 mg, 626.6 μmol, 3 eq) and acetic acid (25.1 mg, 417.7 μmol, 23.9 μL, 2 eq) in MeOH (2 mL) and DCM (2 mL) at 30° C. for 1 hour, then NaBH(OAc)₃ (88.53 mg, 417.70 μmol, 2 eq) was added to the mixture and was stirred at 30° C. for 16 hr. The reaction mixture was quenched by addition 20 mL H₂O at 25° C., and then diluted with 20 mL ethyl acetate and extracted with ethyl acetate (20 mL*3). The combined organic layers were washed with brine 20 mL, dried over drying Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 15%-55% B over 9 min). Compound (3S)-3-[5-[4-[[1-[4-[(1R,2S)-6-hydroxy-2-(2,3,4,5,6-pentadeuteriophenyl)tetralin-1-yl]phenyl]-4-piperidyl]methyl]piperazin-1-yl]-1-oxo-isoindolin-2-yl]piperidine-2,6-dione (56.4 mg, 77.4 μmol, 37. % yield, 100% purity) was obtained as a white solid.

LC-MS (ESI⁺) m/z: 729.4 (M+H)⁺

HPLC: 95.902% purity at 220 nm.

¹H NMR (400 MHZ, DMSO-d₆) δ=8.07 (s, 0.168H), 7.89 (br s, 1H), 7.67 (d, J=8.6 Hz, 1H), 6.95-6.89 (m, 1H), 6.80 (s, 1H), 6.75 (d, J=8.3 Hz, 1H), 6.63 (d, J=2.5 Hz, 1H), 6.54-6.49 (m, 3H), 6.21 (d, J=8.6 Hz, 2H), 5.13 (dd, J=5.2, 13.2 Hz, 1H), 4.37-4.31 (m, 1H), 4.22-4.15 (m, 1H), 4.13 (d, J=4.9 Hz, 1H), 3.52-3.46 (m, 2H), 3.28 (br s, 6H), 2.99-2.92 (m, 2H), 2.83-2.74 (m, 2H), 2.58 (br s, 3H), 2.51 (br d, J=1.5 Hz, 2H), 2.30-2.22 (m, 4H), 2.15-2.10 (m, 3H), 1.78 (br d, J=13.4 Hz, 2H), 1.28 (br d, J=11.8 Hz, 2H).

SFC: retention time, 2.728 min; Area, 94.252%;

### Example 160. Preparation of (I-341) (3S)-3-[5-[4-[[1-[4-[(1S,2R)-6-hydroxy-2-(2,3,4,5,6-pentadeuteriophenyl)tetralin-1-yl]phenyl]-4-piperidyl]methyl]piperazin-1-yl]-1-oxo-isoindolin-2-yl]piperidine-2,6-dione

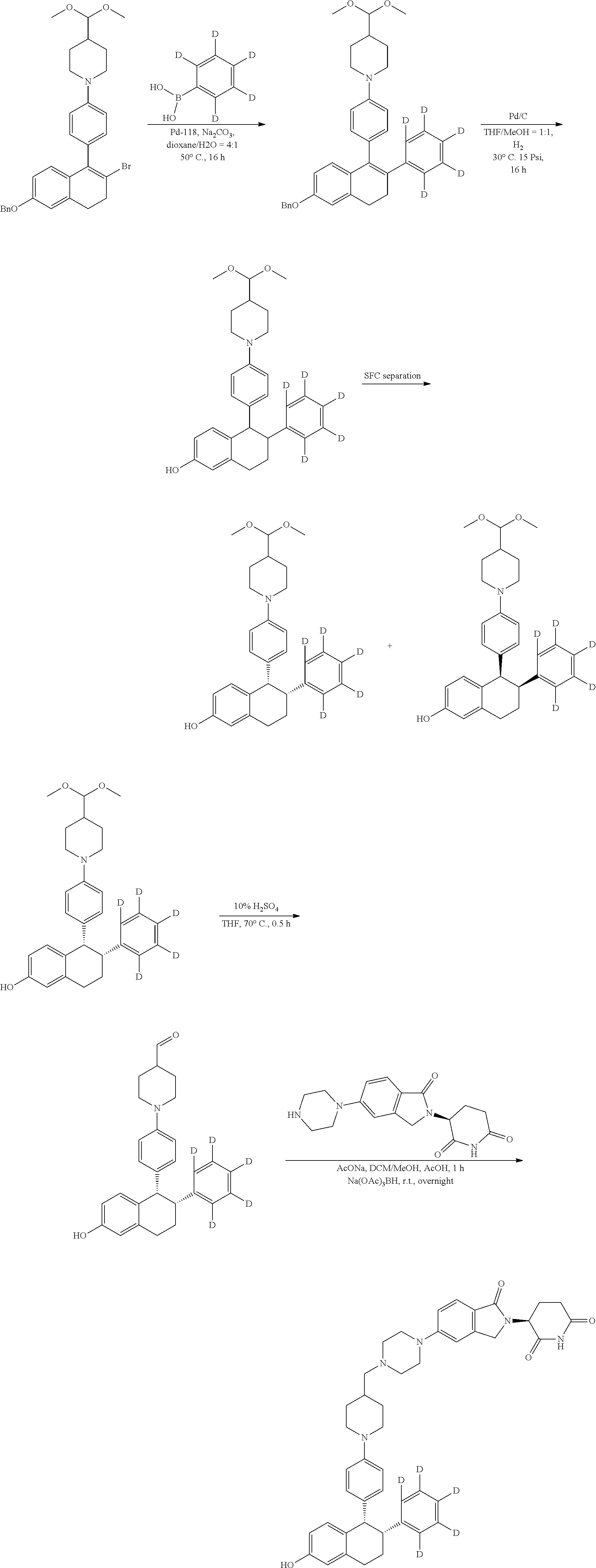

Step 1: A mixture of 1-(4-(6-(benzyloxy)-2-bromo-3,4-dihydronaphthalen-1-yl)phenyl)-4-(dimethoxymethyl)piperidine (400 mg, 729.2 μmol, 1 eq), (2,3,4,5,6-pentadeuteriophenyl)boronic acid (111.1 mg, 875.1 μmol, 1.2 eq), Pd-118 (47.5 mg, 72.9 μmol, 0.1 eq), Na₂CO₃ (231.9 mg, 2.19 mmol, 3 eq) in dioxane (2 mL) and H₂O (0.5 mL) was degassed and purged with N₂ for 3 times, and then the mixture was stirred at 50° C. for 16 hr under N₂ atmosphere. The reaction mixture was quenched by addition 50 mL H₂O at 20° C., and then diluted with 50 mL ethyl acetate and extracted with ethyl acetate (50 mL*3). The combined organic layers were washed with brine 50 mL, dried over drying Na₂SO₄, filtered and concentrated under reduced pressure to give a residue. The residue was purified by flash silica gel chromatography (ISCOR; 12 g SepaFlash® Silica Flash Column, Eluent of 0˜15% Ethyl acetate/Petroleum ether gradient @ 20 mL/min). Compound 1-[4-[6-benzyloxy-2-(2,3,4,5,6-pentadeuteriophenyl)-3,4-dihydronaphthalen-1-yl]phenyl]-4-(dimethoxymethyl)piperidine (330 mg, 599.2 μmol, 82.17% yield) was obtained as a yellow solid.

LC-MS (ESI⁺) m/z: 551.2 (M+H)⁺.

Step 2: To a solution of 1-[4-[6-benzyloxy-2-(2,3,4,5,6-pentadeuteriophenyl)-3,4-dihydronaphthalen-1-yl]phenyl]-4-(dimethoxymethyl)piperidine (330 mg, 599.19 μmol, 1 eq) in THF (3 mL) and MeOH (3 mL) was added Pd/C (10% wt, 330 mg) under N₂ atmosphere. The suspension was degassed and purged with H₂ for 3 times. The mixture was stirred under H₂ (15 psi) at 30° C. for 16 hr. The reaction mixture was filtered and the filter was concentrated. The residue was purified by SFC (column: DAICEL CHIRALCEL OJ (250 mm*30 mm, 10 um); mobile phase: [CO₂-EtOH (0.1% NH₃H₂O)]; B %: 40% %, isocratic elution mode) Rt=3.532/4.309 min. Compound (1R,2S)-1-[4-[4-(dimethoxymethyl)-1-piperidyl]phenyl]-2-(2,3,4,5,6-pentadeuteriophenyl)tetralin-6-ol (103 mg, 211.00 μmol, 35.21% yield, 94.774% purity) and (1S,2R)-1-[4-[4-(dimethoxymethyl)-1-piperidyl]phenyl]-2-(2,3,4,5,6-pentadeuteriophenyl)tetralin-6-ol (100 mg, 207.60 μmol, 34.65% yield, 96.041% purity) were obtained as yellow oil.

LC-MS (ESI⁺) m/z: 463.3 (M+H)⁺

SFC of (5S,6R)-5-(4-(4-(dimethoxymethyl)piperidin-1-yl)phenyl)-6-(phenyl-d5)-5,6,7,8-tetrahydronaphthalen-2-ol: retention time, 3.524 min; Area, 100%;

SFC of (5R,6S)-5-(4-(4-(dimethoxymethyl)piperidin-1-yl)phenyl)-6-(phenyl-d5)-5,6,7,8-tetrahydronaphthalen-2-ol: retention time, 4.290 min; Area, 99.80%;

Step 3: To a solution of (1S,2R)-1-[4-[4-(dimethoxymethyl)-1-piperidyl]phenyl]-2-(2,3,4,5,6-pentadeuteriophenyl)tetralin-6-ol (103 mg, 222.64 μmol, 1 eq) in H₂SO₄ (2 mL) and THF (2 mL). The mixture was stirred at 70° C. for 2 hr. The mixture was alkalied with NaHCO₃ to pH=7˜8. The reaction mixture was diluted with 50 mL H₂O and extracted with ethyl acetate (50 mL*2). The combined organic layers were washed with 50 mL brine, dried over anhydrous Na₂SO₄, filtered and concentrated under reduced pressure to give a residue. The crude product 1-[4-[(1S,2R)-6-hydroxy-2-(2,3,4,5,6-pentadeuteriophenyl)tetralin-1-yl]phenyl]piperidine-4-carbaldehyde (88 mg, 211.25 μmol, 94.89% yield) as yellow oil was used into the next step without further purification.

LC-MS (ESI⁺) m/z: 417.3 (M+H)⁺.

Step 4: A mixture of 1-[4-[1S,2R)-6-hydroxy-2-(2,3,4,5,6-pentadeuteriophenyl)tetralin-1-yl]phenyl]piperidine-4-carbaldehyde (88 mg, 211.2 μmol, 1 eq), (S)-3-(1-oxo-5-(piperazin-1-yl)isoindolin-2-yl)piperidine-2,6-dione benzenesulfonate (154.2 mg, 316.9 μmol, 1.5 eq) and sodium acetate (52 mg, 633.8 μmol, 3 eq) and acetic acid (25.4 mg, 422.5 μmol, 24.2 μL, 2 eq) in MeOH (2 mL) and DCM (2 mL) at 30° C. for 1 hour, then NaBH(OAc)₃ (89.6 mg, 422.5 μmol, 2 eq) was added to the mixture and was stirred at 30° C. for 16 hr. The reaction mixture was quenched by addition 20 mL H₂O at 25° C., and then diluted with 20 mL ethyl acetate and extracted with ethyl acetate (20 mL*3). The combined organic layers were washed with 20 mL brine, dried over Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 15%-55% B over 9 min). Compound (3S)-3-[5-[4-[[1-[4-[(1S,2R)-6-hydroxy-2-(2,3,4,5,6-pentadeuteriophenyl)tetralin-1-yl]phenyl]-4-piperidyl]methyl]piperazin-1-yl]-1-oxo-isoindolin-2-yl]piperidine-2,6-dione (61 mg, 83.13 μmol, 39.35% yield, 99.343% purity) was obtained as a white solid.

LC-MS (ESI⁺) m/z: 729.4 (M+H)⁺

HPLC: 96.695% purity at 220 nm.

¹H NMR (400 MHZ, DMSO-d₆) δ=8.08 (s, 0.185H), 7.91 (s, 1H), 7.67 (d, J=8.6 Hz, 1H), 6.94-6.90 (m, 1H), 6.80 (s, 1H), 6.75 (d, J=8.5 Hz, 1H), 6.63 (d, J=2.5 Hz, 1H), 6.53-6.49 (m, 3H), 6.21 (d, J=8.6 Hz, 2H), 5.13 (dd, J=5.1, 13.2 Hz, 1H), 4.37-4.31 (m, 1H), 4.21-4.16 (m, 1H), 4.13 (d, J=4.8 Hz, 1H), 3.52-3.46 (m, 2H), 3.27 (br d, J=5.2 Hz, 6H), 2.97 (br d, J=4.2 Hz, 2H), 2.84-2.74 (m, 2H), 2.56 (br s, 3H), 2.51 (br s, 2H), 2.25 (br d, J=7.3 Hz, 4H), 2.15-2.10 (m, 3H), 1.78 (br d, J=13.6 Hz, 2H), 1.27 (br d, J=12.5 Hz, 2H)

SFC: retention time, 2.719 min; Area, 93.987%.

### Example 161. Preparation of (I-174) (S)-3-(5-(4-((1-(4-((1R,2S,3S)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

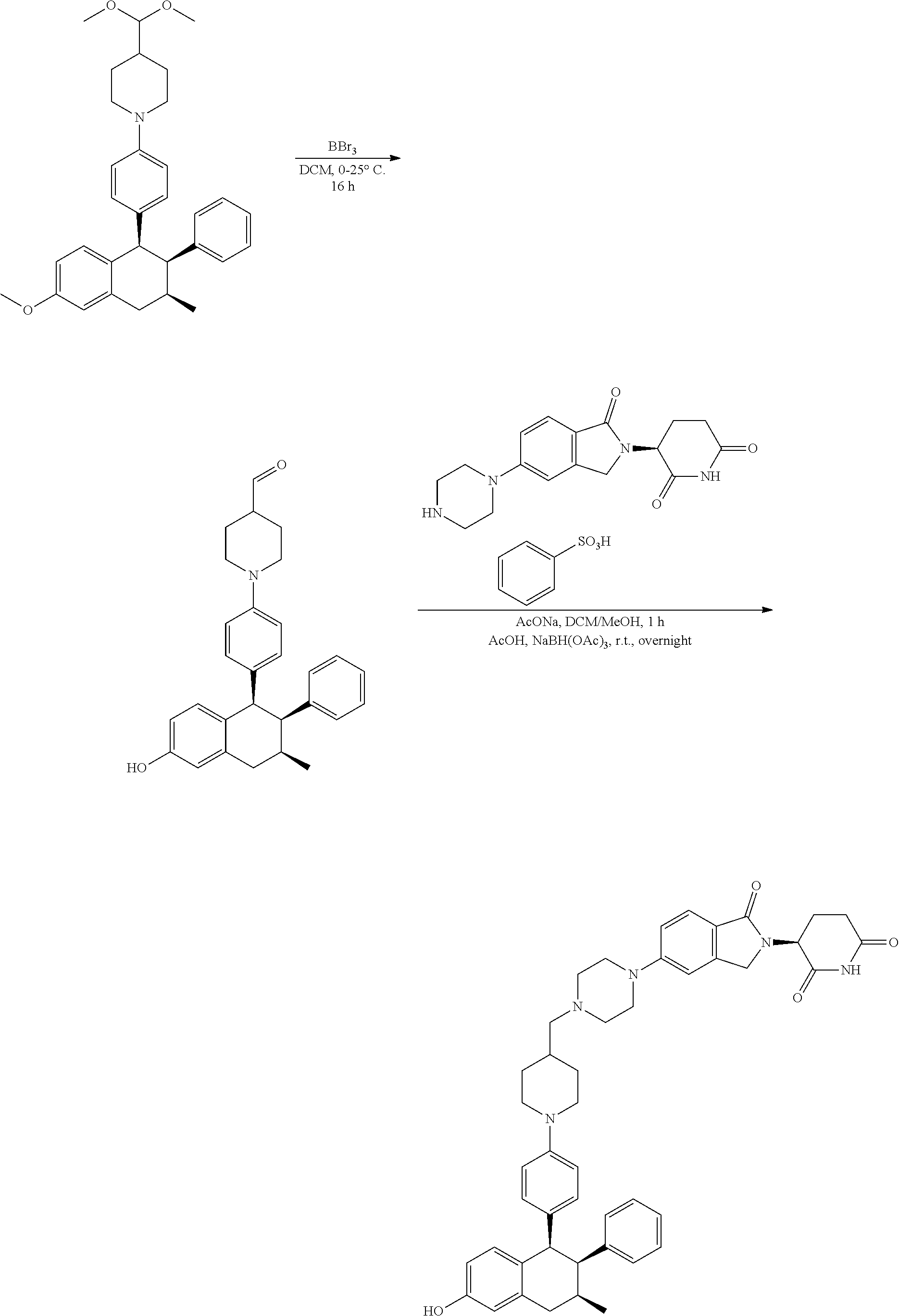

Step 1: To a solution of 4-(dimethoxymethyl)-1-(4-((1R,2S,3S)-6-methoxy-3-methyl-2-phenyl-1,2,3,4-tetrahydronaphthalen-1-yl)phenyl)piperidine (100 mg, 100% Wt, 1 Eq, 206 μmol) in DCM (2 mL) at 0° C. was added tribromoborane (155 mg, 618 μL, 1 molar, 3 Eq, 618 μmol) with N₂. After addition, the mixture was stirred at 0° C. for 4 hour and then the reaction was warmed to 25° C. and was stirred at this temperature for 12 hour. The reaction mixture was quenched with saturated NaHCO₃10 mL and stirred for 10 min at 0° C. Then the mixture was dissolved in water (30 mL) and extracted by ethyl acetate (40 mL). The combined organic layer was dried over Na₂SO₄, filtered and concentrated under vacuum to give 1-(4-((1R,2S,3S)-6-hydroxy-3-methyl-2-phenyl-1,2,3,4-tetrahydronaphthalen-1-yl)phenyl)piperidine-4-carbaldehyde (85 mg, 0.20 mmol, 97%) as a yellow solid. LC-MS (ESI⁺) m/z: 426.2 (M+H)⁺.

Step 2: A mixture of 1-(4-((1R,2S,3S)-6-hydroxy-3-methyl-2-phenyl-1,2,3,4-tetrahydronaphthalen-1-yl)phenyl)piperidine-4-carbaldehyde (85 mg, 1 Eq, 0.20 mmol), (S)-3-(1-oxo-5-(piperazin-1-yl)isoindolin-2-yl)piperidine-2,6-dione benzenesulfonate (0.13 g, 1.3 Eq, 0.26 mmol) and sodium acetate (82 mg, 5 Eq, 1.0 mmol) and acetic acid (36 mg, 34 μL, 3 Eq, 0.60 mmol) in DCM (2 mL) and MeOH (2 mL) at 25° C. for 1 hour, then sodium triacetoxyborohydride (85 mg, 2 Eq, 0.40 mmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*30 mm*3 um mobile phase: [water (FA)-ACN]; B %: 21%-51%, 7 min). The aqueous phase was lyophilized to dryness to give (S)-3-(5-(4-((1-(4-((1R,2S,3S)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (60 mg, 81 μmol, 41%, 99.509% Purity) as a white solid.

LC-MS (ESI⁺) m/z: 738.3 (M+H)⁺.

HPLC: 99.509%, purity at 220 nm.

SFC: retention time, 2.561 min; Area, 91.502%;

¹H NMR (400 MHZ, DMSO-d₆) δ=10.96 (s, 1H), 9.24-9.09 (m, 1H), 8.14 (s, 0.368H), 7.52 (d, J=8.7 Hz, 1H), 7.12-7.02 (m, 5H), 6.90-6.73 (m, 4H), 6.66-6.56 (m, 4H), 6.49 (dd, J=2.4, 8.4 Hz, 1H), 5.05 (dd, J=5.1, 13.2 Hz, 1H), 4.45 (br d, J=5.0 Hz, 1H), 4.38-4.27 (m, 1H), 4.25-4.16 (m, 1H), 3.61-3.44 (m, 3H), 3.28 (br s, 5H), 3.11 (br d, J=4.5 Hz, 1H), 2.98-2.83 (m, 1H), 2.71 (br d, J=12.4 Hz, 1H), 2.63-2.51 (m, 4H), 2.42 (br d, J=3.0 Hz, 3H), 2.40-2.28 (m, 1H), 2.20 (br d, J=6.9 Hz, 2H), 2.01-1.91 (m, 1H), 1.76 (br d, J=11.6 Hz, 2H), 1.69-1.57 (m, 1H), 1.25-1.08 (m, 2H), 0.72 (br d, J=6.1 Hz, 3H).

### Example 162. Preparation of (I-334) (S)-3-(5-(4-((1-(4-((1S,2R,3S)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

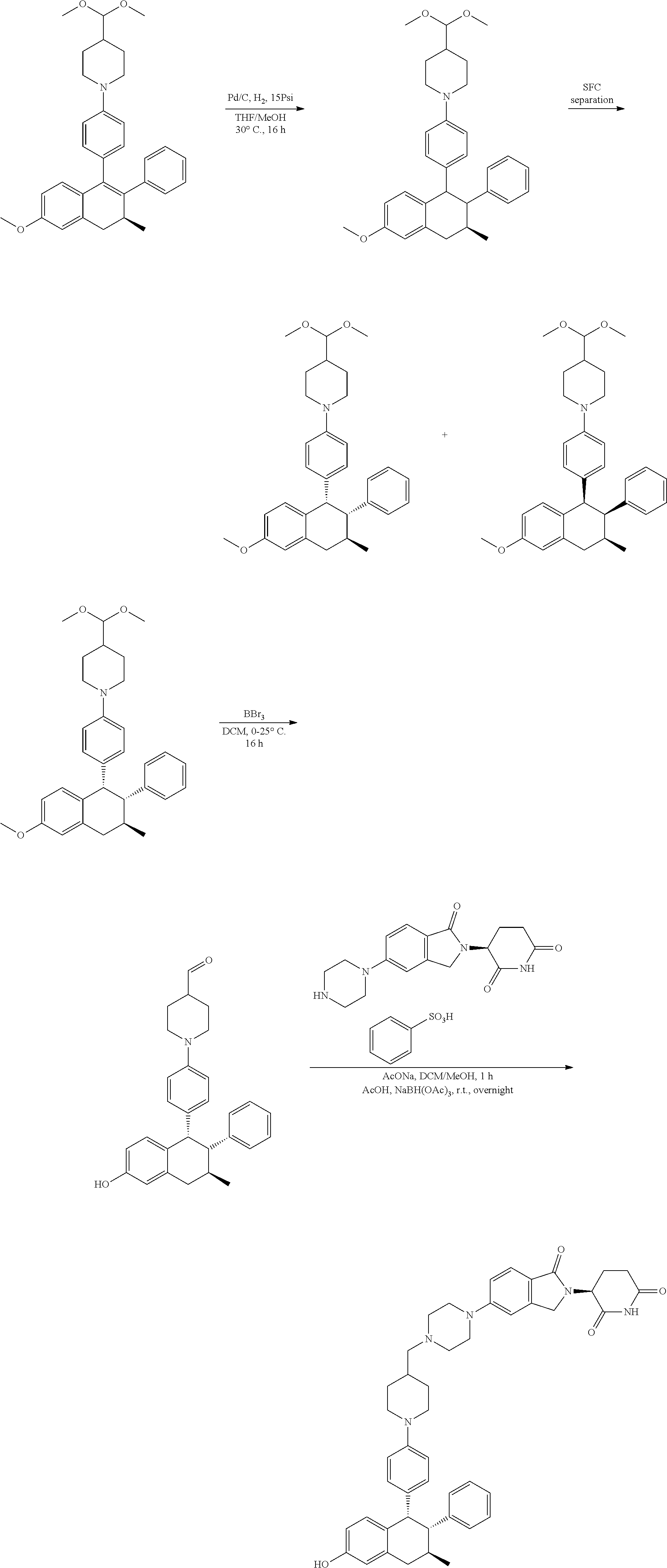

Step 1: To a solution of 4-(dimethoxymethyl)-1-[4-[(3S)-6-methoxy-3-methyl-2-phenyl-3,4-dihydronaphthalen-1-yl]phenyl]piperidine (520 mg, 1.08 mmol, 1 eq) in THF (10 mL) and MeOH (10 mL) was added Pd/C (10%, 500 mg) under N₂ atmosphere. The suspension was degassed and purged with H₂ for 3 times. The mixture was stirred under H₂ (15 Psi) at 30° C. for 16 hr. The reaction mixture was filtered and the filter was concentrated. to give 4-(dimethoxymethyl)-1-[4-[(3S)-6-methoxy-3-methyl-2-phenyl-tetralin-1-yl]phenyl]piperidine (490 mg, 1.01 mmol, 93.84% yield) as a white solid. LC-MS (ESI⁺) m/z: 486.2 (M+H)⁺.

Step 2: The white solid was subjected by SFC: Column: DAICEL CHIRALPAK AD (250 mm*30 mm, 10 um); Mobile phase: A: Supercritical CO₂, B: MeOH (0.1% NH₃H₂O), A:B=35:35 at 80 mL/min. The pure fractions were collected respectively and the solvents were evaporated under vacuum to give the product 4-(dimethoxymethyl)-1-(4-((1R,2S,3S)-6-methoxy-3-methyl-2-phenyl-1,2,3,4-tetrahydronaphthalen-1-yl)phenyl)piperidine (250 mg, 515 μmol, 75.8%, 100% Purity) and 4-(dimethoxymethyl)-1-(4-((1S,2R,3S)-6-methoxy-3-methyl-2-phenyl-1,2,3,4-tetrahydronaphthalen-1-yl)phenyl)piperidine (30 mg, 60 μmol, 8.9%, 97.504% purity) as a white solid. LC-MS (ESI⁺) m/z: 486.2 (M+H)⁺.

Step 3: To a solution of 4-(dimethoxymethyl)-1-(4-((1S,2R,3S)-6-methoxy-3-methyl-2-phenyl-1,2,3,4-tetrahydronaphthalen-1-yl)phenyl)piperidine (30 mg, 1 Eq, 60 μmol) in DCM (2 mL) at 0° C. was added tribromoborane (45 mg, 0.18 mL, 1 molar, 3 Eq. 0.18 mmol) with N₂. After addition, the mixture was stirred at 0° C. for 4 hour and then the reaction was warmed to 25° C. and was stirred at this temperature for 12 hour. The reaction mixture was quenched with saturated NaHCO₃ (10 mL) and stirred for 10 min at 0° C. Then the mixture was dissolved in water (30 mL) and extracted by ethyl acetate (40 mL). The combined organic layer was dried over Na₂SO₄, filtered and concentrated under vacuum to give 1-(4-((1S,2R,3S)-6-hydroxy-3-methyl-2-phenyl-1,2,3,4-tetrahydronaphthalen-1-yl)phenyl)piperidine-4-carbaldehyde (25 mg, 59 μmol, 98%) as a yellow solid. LC-MS (ESI⁺) m/z: 426.2 (M+H)⁺.

Step 4: A mixture of 1-(4-((1S,2R,3S)-6-hydroxy-3-methyl-2-phenyl-1,2,3,4-tetrahydronaphthalen-1-yl)phenyl)piperidine-4-carbaldehyde (25 mg, 1 Eq, 59 μmol), (S)-3-(1-oxo-5-(piperazin-1-yl)isoindolin-2-yl)piperidine-2,6-dione benzenesulfonate (37 mg, 1.3 Eq, 76 μmol) and sodium acetate (24 mg, 5 Eq, 0.29 mmol) and acetic acid (11 mg, 10 μL, 3 Eq, 0.18 mmol) in DCM (2 mL) and MeOH (2 mL) at 25° C. for 1 hour, then sodium triacetoxyborohydride (25 mg, 2 Eq, 0.12 mmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*30 mm*3 um mobile phase: [water (FA)-ACN]; B %: 22%-52%, 7 min). The aqueous phase was lyophilized to dryness to give (S)-3-(5-(4-((1-(4-((1S,2R,3S)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (5.9 mg, 8.0 μmol, 14%, 99.983% Purity) as a pink solid.

LC-MS (ESI⁺) m/z: 738.3 (M+H)⁺

HPLC: 99.983%, purity at 220 nm.

SFC: retention time, 3.165 min; Area, 93.965%;

¹H NMR (400 MHZ, DMSO-d₆) δ=10.96 (s, 1H), 9.11 (br s, 1H), 8.17 (s, 0.182H), 7.51 (d, J=8.5 Hz, 1H), 7.15-6.97 (m, 5H), 6.70-6.52 (m, 6H), 6.45 (dd, J=2.1, 8.2 Hz, 1H), 6.23 (br d, J=8.6 Hz, 2H), 5.05 (dd, J=5.1, 13.2 Hz, 1H), 4.37-4.28 (m, 1H), 4.25-4.15 (m, 1H), 3.95 (d, J=5.0 Hz, 1H), 3.60-3.48 (m, 2H), 3.28 (br s, 6H), 3.08 (br dd, J=5.4, 17.4 Hz, 1H), 3.00-2.84 (m, 2H), 2.64-2.51 (m, 6H), 2.43-2.30 (m, 2H), 2.20 (br d, J=7.0 Hz, 2H), 2.02-1.90 (m, 1H), 1.76 (br d, J=11.9 Hz, 2H), 1.65 (br d, J=3.3 Hz, 1H), 1.27-1.10 (m, 2H), 0.73 (d, J=6.2 Hz, 3H).

### Example 163. Preparation of (I-175) (S)-3-(5-(4-((1-(4-((1R,2S,3R)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

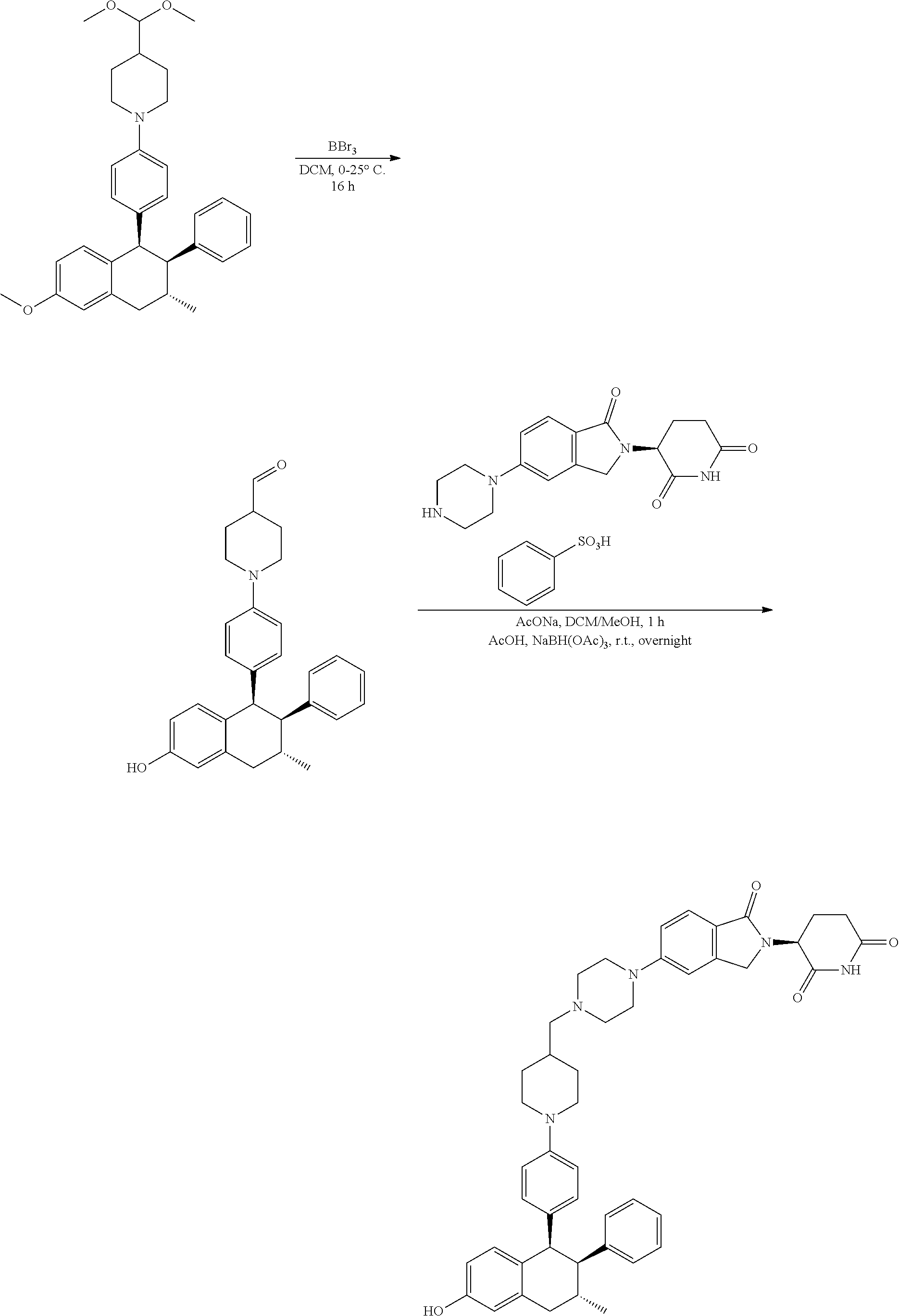

Step 1: To a solution of 4-(dimethoxymethyl)-1-(4-((1R,2S,3R)-6-methoxy-3-methyl-2-phenyl-1,2,3,4-tetrahydronaphthalen-1-yl)phenyl)piperidine (35 mg, 94.264% Wt, 1 Eq, 68 μmol) in DCM (2 mL) at 0° C. was added tribromoborane (51 mg, 0.20 mL, 1 molar, 3 Eq, 0.20 mmol) with N₂. After addition, the mixture was stirred at 0° C. for 4 hour and then the reaction was warmed to 25° C. and was stirred at this temperature for 12 hour. The reaction mixture was quenched with saturated NaHCO₃10 mL and stirred for 10 min at 0° C. Then the mixture was dissolved in water (30 mL) and extracted by ethyl acetate (40 mL). The combined organic layer was dried over Na₂SO₄, filtered and concentrated under vacuum to give 1-(4-((1R,2S,3R)-6-hydroxy-3-methyl-2-phenyl-1,2,3,4-tetrahydronaphthalen-1-yl)phenyl)piperidine-4-carbaldehyde (25 mg, 59 μmol, 86%) as a yellow solid. LC-MS (ESI⁺) m/z: 426.2 (M+H)⁺.

Step 2: A mixture of 1-(4-((1R,2S,3R)-6-hydroxy-3-methyl-2-phenyl-1,2,3,4-tetrahydronaphthalen-1-yl)phenyl)piperidine-4-carbaldehyde (25 mg, 1 Eq, 59 μmol), (S)-3-(1-oxo-5-(piperazin-1-yl)isoindolin-2-yl)piperidine-2,6-dione benzenesulfonate (37 mg, 1.3 Eq, 76 μmol) and sodium acetate (24 mg, 5 Eq, 0.29 mmol) and acetic acid (11 mg, 10 μL, 3 Eq, 0.18 mmol) in DCM (2 mL) and MeOH (2 mL) at 25° C. for 1 hour, then sodium triacetoxyborohydride (25 mg, 2 Eq, 0.12 mmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*30 mm*3 um mobile phase: [water (FA)-ACN]; B %: 20%-50%, 7 min). The aqueous phase was lyophilized to dryness to give (S)-3-(5-(4-((1-(4-((1R,2S,3R)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (18.4 mg, 24.8 μmol, 42%, 99.513% purity) as a pink solid.

LC-MS (ESI+) m/z: 738.3 (M+H)⁺

HPLC: 99.513%, purity at 220 nm.

SFC: retention time, 2.731 min; Area, 94.767%;

¹H NMR (400 MHZ, DMSO-d₆) δ=10.96 (s, 1H), 9.11 (br d, J=0.7 Hz, 1H), 8.15 (s, 0.608H), 7.52 (d, J=8.6 Hz, 1H), 7.15-7.01 (m, 5H), 6.71-6.55 (m, 6H), 6.46 (dd, J=2.4, 8.2 Hz, 1H), 6.24 (d, J=8.6 Hz, 2H), 5.05 (dd, J=4.9, 13.3 Hz, 1H), 4.40-4.29 (m, 1H), 4.25-4.14 (m. 1H), 3.96 (d, J=4.9 Hz, 1H), 3.63-3.45 (m, 3H), 3.32-3.20 (m, 8H), 3.09 (br dd, J=5.4, 17.5 Hz, 1H), 3.00-2.85 (m, 2H), 2.70-2.54 (m, 3H), 2.42-2.29 (m, 2H), 2.26-2.17 (m, 2H), 2.04-1.90 (m, 1H), 1.77 (br d, J=11.8 Hz, 2H), 1.71-1.60 (m, 1H), 1.26-1.09 (m, 2H), 0.74 (d, J=6.2 Hz, 3H).

### Example 164. Preparation of (I-333) (S)-3-(5-(4-((1-(4-((1S,2R,3R)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

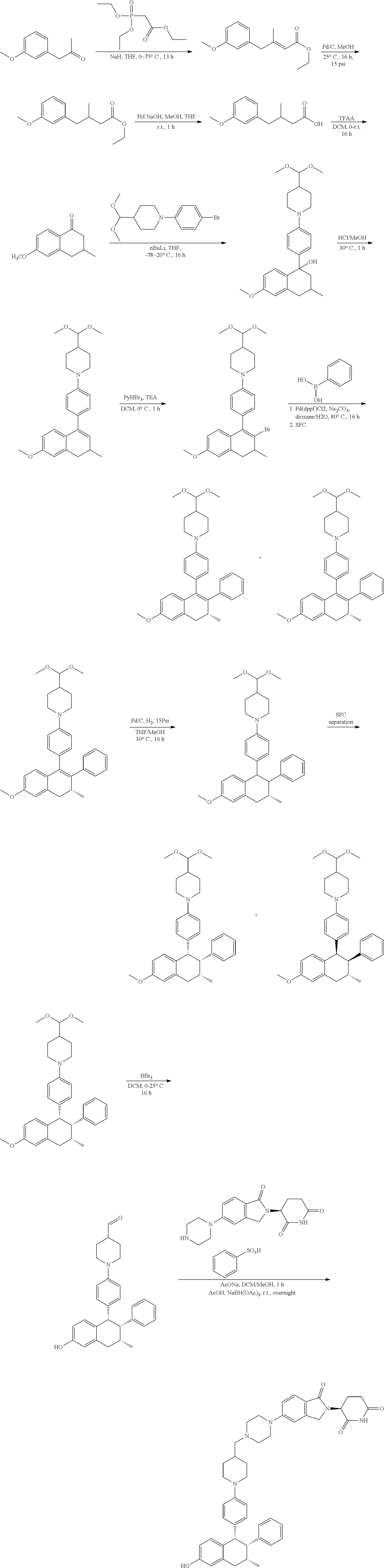

Step 1: To a solution of NaH (3.65 g, 91.35 mmol, 60% purity, 1.5 eq) in THF (300 mL) degassed and purged with N₂ for 3 times then the mixture was added ethyl 2-(diethoxyphosphoryl)acetate (16.38 g, 73.08 mmol, 14.50 mL, 1.2 eq) stirred at 0° C. for 0.5 h under N₂ atmosphere. Then the 1-(3-methoxyphenyl) propan-2-one (10 g, 60.90 mmol, 1 eq) was added to the mixture. The mixture was stirred at 0˜75° C. for 13 hr under N₂ atmosphere. Then the mixture was cooled to room temperature. The reaction was quenched with H₂O (200 mL) under N₂ at ice bath, the mixture was extracted with EtOAc (300 mL). The combined extracts was dried over anhydrous Na₂SO₄, filtered and concentrated to dryness in vacuo to give a yellow oil. The yellow oil was purified by flash column (petroleum ether/ethyl acetate from 100/0 to 85/15) and the organic layer was concentrated in vacuo to give ethyl(E)-4-(3-methoxyphenyl)-3-methylbut-2-enoate (9.35 g, 37.11 mmol, 60.94% yield, 93% purity) as a colorless oil. LC-MS (ESI⁺) m/z: 235.2 (M+H)⁺

Step 2: To a solution of ethyl(E)-4-(3-methoxyphenyl)-3-methyl-but-2-enoate (9.35 g, 37.11 mmol, 1 eq) in MeOH (100 mL) was added Pd/C (10%, 8.7 g) under N₂ atmosphere. The suspension was degassed and purged with H₂ for 3 times. The mixture was stirred under H₂ (15 Psi) at 25° C. for 16 hr. The reaction mixture was filtered and the filter was concentrated. The crude product ethyl 4-(3-methoxyphenyl)-3-methylbutanoate (8.5 g, 35.97 mmol, 96.92% yield) as a colorless oil was used into the next step without further purification.

Step 3: To a solution of ethyl 4-(3-methoxyphenyl)-3-methyl-butanoate (8.5 g, 35.97 mmol, 1 eq) in MeOH (25 mL) and THF (25 mL) was added NaOH (3 M, 47.96 mL, 4 eq). The mixture was stirred at 20° C. for 1 hr. The mixture was acidified with HCl (1M) to PH=5˜6. To the mixture was added water (100 mL), then was extracted with ethyl acetate 100 mL (50 mL*2). The combined organic layers were washed with brine (50 mL), dried over Na₂SO₄, filtered and concentrated under reduced pressure to give the crude product 4-(3-methoxyphenyl)-3-methylbutanoic acid (7.4 g, 35.53 mmol, 98.79% yield) as colorless oil was used into the next step without further purification.

Step 4: To a solution of 4-(3-methoxyphenyl)-3-methyl-butanoic acid (7.4 g, 35.53 mmol, 1 eq) in DCM (100 mL) was added (2,2,2-trifluoroacetyl) 2,2,2-trifluoroacetate (37.32 g, 177.67 mmol, 24.70 mL, 5 eq) at 0° C. under N₂ atmosphere, and then the mixture was stirred at 0˜20° C. for 16 hr. The reaction mixture was partitioned between H₂O 100 mL and DCM 100 mL. The organic phase was separated, washed with DCM (100 mL*3), dried over drying Na₂SO₄, filtered and concentrated under reduced pressure to give a residue. The residue was subjected to column chromatography over silica gel (gradient elution: 0˜15% ethyl acetate in petroleum ether). The pure fractions were collected and concentrated to dryness in vacuo to give 6-methoxy-3-methyl-3,4-dihydronaphthalen-1 (2H)-one (3.7 g, 18.67 mmol, 52.55% yield, 96% purity) as a yellow oil.

LC-MS (ESI⁺) m/z: 190.8 (M+H)⁺.

¹H NMR (400 MHz, CDCl₃) δ=8.03 (d, J=8.8 Hz, 1H), 6.85 (dd, J=2.3, 8.8 Hz, 1H), 6.73 (s, 1H), 3.89 (s, 3H), 2.99-2.93 (m, 1H), 2.85-2.63 (m, 2H), 2.42-2.30 (m, 2H), 1.15 (d, J=6.0 Hz, 3H).

Step 5: To a solution of 1-(4-bromophenyl)-4-(dimethoxymethyl)piperidine (5.95 g, 18.92 mmol, 1.8 eq) in THF (70 mL) was added dropwise n-BuLi (2.5 M, 6.73 mL, 1.6 eq) at −78° C. for 1 h and then 6-methoxy-3-methyl-tetralin-1-one (2 g, 10.51 mmol, 1 eq) in THF (70 mL) was added dropwise at −78° C. The resulting mixture was stirred at −78˜25° C. for 16 h. The reaction mixture was quenched by addition 200 mL H₂O at 25° C. and then extracted with ethyl acetate (100 mL*3). The combined organic layers were washed with brine 100 mL, dried over drying Na₂SO₄, filtered and concentrated under reduced pressure to give a residue. The residue was subjected to column chromatography over silica gel (gradient elution: 0˜25% ethyl acetate in petroleum ether). The pure fractions were collected and concentrated to dryness in vacuo to give 1-(4-(4-(dimethoxymethyl)piperidin-1-yl)phenyl)-6-methoxy-3-methyl-1,2,3,4-tetrahydronaphthalen-1-ol (2.3 g, 5.40 mmol, 51.41% yield) as a yellow oil. LC-MS (ESI⁺) m/z: 426.3 (M+H)⁺.

Step 6: To a solution of 1-[4-[4-(dimethoxymethyl)-1-piperidyl]phenyl]-6-methoxy-3-methyl-tetralin-1-ol (2 g, 4.70 mmol, 1 eq) in HCl/MeOH (20 mL). The mixture was stirred at 30° C. for 1 hr. The mixture was adjusted with NaHCO₃ to pH=7˜8. The reaction mixture was diluted with 50 mL H₂O and extracted with ethyl acetate (50 mL*2). The combined organic layers were washed with brine 50 mL, dried over Na₂SO₄, filtered and concentrated under reduced pressure to give a residue. The residue was subjected to column chromatography over silica gel (gradient elution: 0˜15% ethyl acetate in petroleum ether). The pure fractions were collected and concentrated to dryness in vacuo to give 4-(dimethoxymethyl)-1-[4-(6-methoxy-3-methyl-3,4-dihydronaphthalen-1-yl)phenyl]piperidine (1.8 g, 4.42 mmol, 93.98% yield) as a yellow solid. LC-MS (ESI⁺) m/z: 408.3 (M+H)⁺.

Step 7: A mixture of 4-(dimethoxymethyl)-1-[4-(6-methoxy-3-methyl-3,4-dihydronaphthalen-1-yl)phenyl]piperidine (1.8 g, 4.42 mmol, 1 eq), BLAH;pyridin-1-ium (1.41 g, 4.42 mmol, 1 eq), TEA (670.38 mg, 6.63 mmol, 922.12 μL, 1.5 eq) in DCM (30 mL) was degassed and purged with N₂ for 3 times and then the mixture was stirred at 0° C. for 1 hr under N₂ atmosphere. The reaction mixture was quenched by addition 100 mL H₂O at 25° C. and then extracted with ethyl acetate (100 mL*3). The combined organic layers were washed with 100 mL brine, dried over drying Na₂SO₄, filtered and concentrated under reduced pressure to give a residue. The residue was subjected to column chromatography over silica gel (gradient elution: 0˜20% ethyl acetate in petroleum ether). The pure fractions were collected and concentrated to dryness in vacuo to give 1-[4-(2-bromo-6-methoxy-3-methyl-3,4-dihydronaphthalen-1-yl)phenyl]-4-(dimethoxymethyl)piperidine (1.7 g, 3.49 mmol, 79.13% yield) as a yellow oil. LC-MS (ESI⁺) m/z: 488.2 (M+H)⁺.

Step 8: A mixture of 1-[4-(2-bromo-6-methoxy-3-methyl-3,4-dihydronaphthalen-1-yl)phenyl]-4-(dimethoxymethyl)piperidine (1.7 g, 3.49 mmol, 1 eq), phenylboronic acid (511.34 mg, 4.19 mmol, 1.2 eq), disodium; carbonate (1.11 g, 10.48 mmol, 3 eq), cyclopentyl(diphenyl)phosphane;dichloromethane;dichloropalladium;iron (285.40 mg, 349.48 μmol, 0.1 eq) in dioxane (20 mL) and H₂O (5 mL) was degassed and purged with N₂ for 3 times and then the mixture was stirred at 80° C. for 16 hr under N₂ atmosphere. The reaction mixture was diluted with H₂O 100 mL and extracted with ethyl acetate (100 mL*2). The combined organic layers were washed with brine 100 mL, dried over Na₂SO₄, filtered and concentrated under reduced pressure to give a residue. The residue was purified by flash silica gel chromatography (ISCO®; 20 g SepaFlash® Silica Flash Column, Eluent of 0˜20% Ethyl acetate/Petroleum ether gradient @ 40 mL/min) to give desired compound (1.2 g, yield 68.53%, purity 93.6%) as a yellow gum, which was further separated by SFC (column: DAICEL CHIRALPAK AD (250 mm*30 mm, 10 um); mobile phase: [CO₂-EtOH (0.1% NH₃H₂O)]; B %: 25%, isocratic elution mode). RT=4.869/5.243 min. The pure fractions were collected and concentrated to dryness in vacuo to give 4-(dimethoxymethyl)-1-[4-[(3R)-6-methoxy-3-methyl-2-phenyl-3,4-dihydronaphthalen-1-yl]phenyl]piperidine (490 mg, 1.01 mmol, 28.79% yield, 99.304% purity) and 4-(dimethoxymethyl)-1-[4-[(3S)-6-methoxy-3-methyl-2-phenyl-3,4-dihydronaphthalen-1-yl]phenyl]piperidine (520 mg, 1.04 mmol, 29.70% yield, 96.528% purity) were obtained as white solid. LC-MS (ESI⁺) m/z: 484.2 (M+H)⁺.

Step 9: To a solution of 4-(dimethoxymethyl)-1-[4-[(3R)-6-methoxy-3-methyl-2-phenyl-3,4-dihydronaphthalen-1-yl]phenyl]piperidine (490 mg, 1.01 mmol, 1 eq) in THF (10 mL) and MeOH (10 mL) was added Pd/C (10%, 500 mg) under N₂ atmosphere. The suspension was degassed and purged with H₂ for 3 times. The mixture was stirred under H₂ (15 Psi) at 30° C. for 16 hr. The reaction mixture was filtered and the filter was concentrated. to give 4-(dimethoxymethyl)-1-[4-[(3R)-6-methoxy-3-methyl-2-phenyl-tetralin-1-yl]phenyl]piperidine (480 mg, 988.36 μmol, 97.55% yield) as a white solid. LC-MS (ESI⁺) m/z: 486.2 (M+H)⁺.

Step 10: The white solid was subjected by SFC: Column: DAICEL CHIRALPAK OJ (250 mm*30 mm, 10 um); Mobile phase: A: Supercritical CO₂, B: MeOH (0.1% NH3 H2O), A:B=35:35 at 80 mL/min. The pure fractions were collected respectively and the solvents were evaporated under vacuum to give the product 4-(dimethoxymethyl)-1-(4-((1S,2R,3R)-6-methoxy-3-methyl-2-phenyl-1,2,3,4-tetrahydronaphthalen-1-yl)phenyl)piperidine (250 mg, 506 μmol, 70.3%, 98.394% Purity) and 4-(dimethoxymethyl)-1-(4-((1R,2S,3R)-6-methoxy-3-methyl-2-phenyl-1,2,3,4-tetrahydronaphthalen-1-yl)phenyl)piperidine (35 mg, 68 μmol, 9.4%, 94.264% Purity) as a white solid. LC-MS (ESI⁺) m/z: 486.2 (M+H)⁺.

Step 11: To a solution of 4-(dimethoxymethyl)-1-(4-((1S,2R,3R)-6-methoxy-3-methyl-2-phenyl-1,2,3,4-tetrahydronaphthalen-1-yl)phenyl)piperidine (100 mg, 1 Eq, 203 μmol) in DCM (2 mL) at 0° C. was added tribromoborane (152 mg, 608 μL, 1 molar, 3 Eq, 608 μmol) with N₂. After addition, the mixture was stirred at 0° C. for 4 hour and then the reaction was warmed to 25° C. and was stirred at this temperature for 12 hour. The reaction mixture was quenched with saturated 10 mL NaHCO₃ and stirred for 10 min at 0° C. Then the mixture was dissolved in water (30 mL) and extracted by ethyl acetate (40 mL). The combined organic layer was dried over Na₂SO₄, filtered and concentrated under vacuum to give 1-(4-((1S,2R,3R)-6-hydroxy-3-methyl-2-phenyl-1,2,3,4-tetrahydronaphthalen-1-yl)phenyl)piperidine-4-carbaldehyde (85 mg, 0.20 mmol, 99%) as a yellow solid. LC-MS (ESI⁺) m/z: 426.2 (M+H)⁺.

Step 12: A mixture of 1-(4-((1S,2R,3R)-6-hydroxy-3-methyl-2-phenyl-1,2,3,4-tetrahydronaphthalen-1-yl)phenyl)piperidine-4-carbaldehyde (85 mg, 1 Eq, 0.20 mmol), (S)-3-(1-oxo-5-(piperazin-1-yl)isoindolin-2-yl)piperidine-2,6-dione benzenesulfonate (0.13 g, 1.3 Eq, 0.26 mmol) and sodium acetate (82 mg, 5 Eq, 1.0 mmol) and acetic acid (36 mg, 34 μL, 3 Eq, 0.60 mmol) in DCM (2 mL) and MeOH (2 mL) at 25° C. for 1 hour, then sodium triacetoxyborohydride (85 mg, 2 Eq, 0.40 mmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep. HPLC (column: Welch Xtimate C18 150*30 mm*3 um mobile phase: [water (FA)-ACN]; B %: 21%-51%, 7 min). The aqueous phase was lyophilized to dryness to give (S)-3-(5-(4-((1-(4-((1S,2R,3R)-6-hydroxy-3-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (69.6 mg, 93.9 μmol, 47%, 99.551% purity) as a pink solid.

LC-MS (ESI⁺) m/z: 738.3 (M+H)⁺

HPLC: 99.551%, purity at 220 nm.

SFC: retention time, 3.123 min; Area, 90.089%;

¹H NMR (400 MHZ, DMSO-d₆) δ=11.02 (s, 1H), 9.31-9.14 (m, 1H), 8.20 (s, 0.315H), 7.58 (d, J=8.9 Hz, 1H), 7.17-7.08 (m, 5H), 6.97-6.79 (m, 4H), 6.72-6.62 (m, 4H), 6.55 (dd, J=2.4, 8.4 Hz, 1H), 5.11 (dd, J=5.1, 13.3 Hz, 1H), 4.51 (br d, J=5.0 Hz, 1H), 4.42-4.34 (m, 1H), 4.30-4.21 (m, 1H), 3.66-3.51 (m, 3H), 3.34 (br s, 5H), 3.17 (br d, J=4.4 Hz, 1H), 3.03-2.89 (m, 1H), 2.77 (br d, J=12.4 Hz, 1H), 2.68-2.57 (m, 4H), 2.47 (br d, J=4.4 Hz, 3H), 2.42 (br dd, J=4.5, 13.3 Hz, 1H), 2.26 (br d, J=6.8 Hz, 2H), 2.07-1.96 (m, 1H), 1.82 (br d, J=11.6 Hz, 2H), 1.75-1.62 (m, 1H), 1.31-1.14 (m, 2H), 0.78 (br d, J=6.1 Hz, 3H).

### Example 165. Preparation of (I-422) 3-((S)-7-(4-((1-(4-((1R,2S)-6-hydroxy-2-phenyl-1,2,3,4-tetrahydronaphthalen-1-yl)phenyl)piperidin-4-yl)methyl)piperazin-1-yl)-5,6,7,8-tetrahydronaphthalen-2-yl)piperidine-2,6-dione and (I-423) 3-((R)-7-(4-((1-(4-((1R,2S)-6-hydroxy-2-phenyl-1,2,3,4-tetrahydronaphthalen-1-yl)phenyl)piperidin-4-yl)methyl)piperazin-1-yl)-5,6,7,8-tetrahydronaphthalen-2-yl)piperidine-2,6-dione

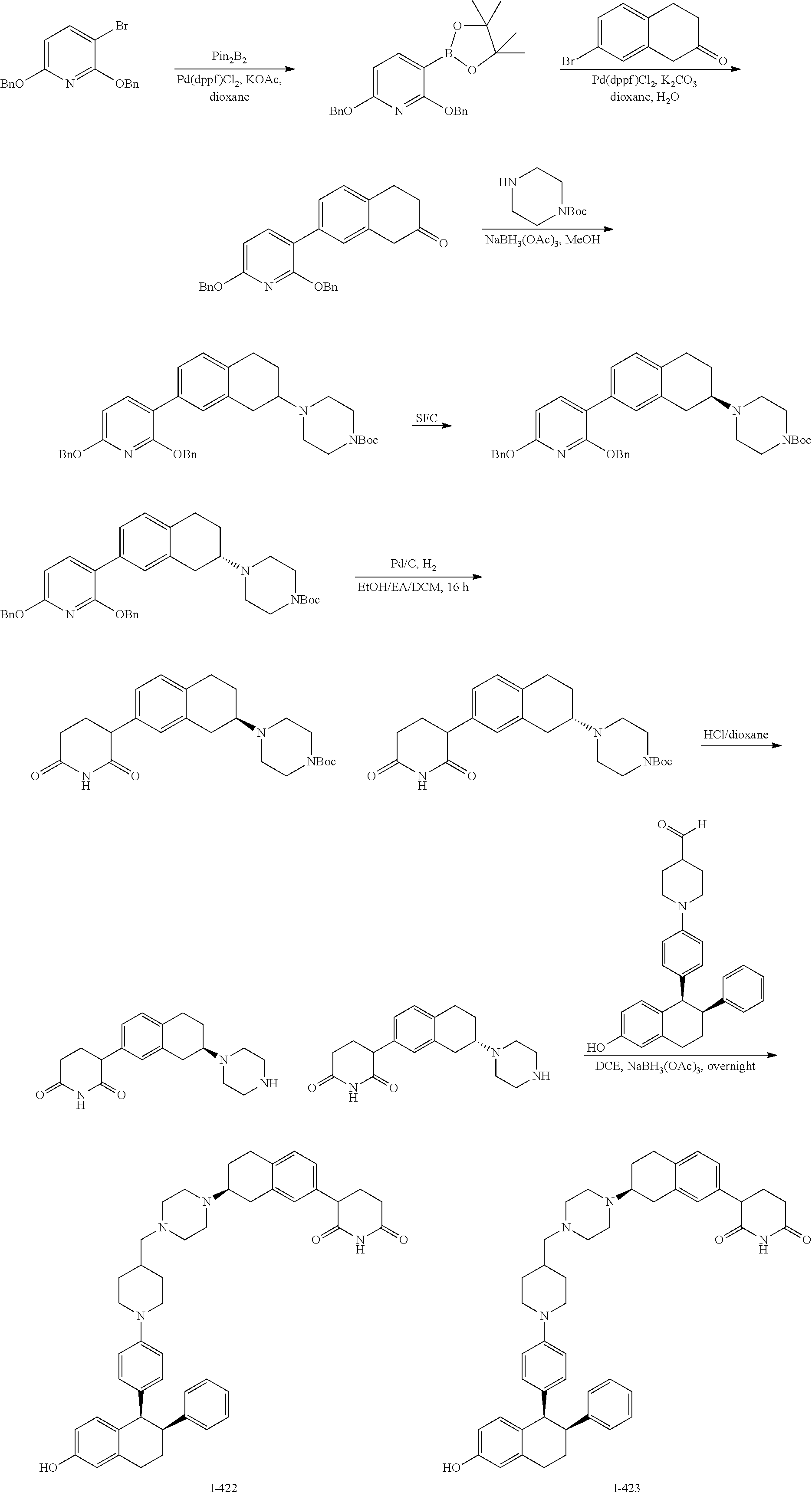

Step 1: A mixture of 2,6-bis(benzyloxy)-3-bromopyridine (4.00 g, 10.8 mmol, 1.0 eq.), Potassium acetate (3.18 g, 2.03 mL, 3 eq., 32.4 mmol), Bis(pinacolato)diborane (5.49 g, 21.6 mmol, 2.0 eq.) and PdCl₂(dppf) (791 mg, 1.08 mmol, 0.1 eq.) in Dioxane (100 mL) was degassed and purged with N₂ for 3 times, and then the mixture was stirred at 90° C. for 12 hour under N₂ atmosphere. LCMS showed the reaction was completed. The reaction mixture was quenched by addition EA 10 mL at 20° C., and then diluted with water 10 mL and extracted with EA 90 mL (30 mL×3). The combined organic layers were washed with EA 15 mL (5 mL×3), dried over Na₂SO₄, filtered and concentrated under reduced pressure to give a residue. The crude material was purified on silica gel column chromatography. (from pure PE to PE/EtOAc=I/O) to give 2,6-bis(benzyloxy)-3-(4,4,5,5-tetramethyl-1,3,2-dioxaborolan-2-yl)pyridine (1.8 g, 4.3 mmol, 40%) as a yellow solid. LC-MS (ESI⁺) m/z: 418.2 (M+H)⁺.

Step 2: A mixture of 7-bromo-3,4-dihydronaphthalen-2 (1H)-one (0.9 g, 4.00 mmol, 1.0 eq.), 2,6-bis(benzyloxy)-3-(4,4,5,5-tetramethyl-1,3,2-dioxaborolan-2-yl)pyridine (2.10 g, 5.03 mmol, 1.26 eq.), PdCl₂(dppf) (293 mg, 400 μmol, 0.1 eq.) and K₂CO₃ (1.66 g, 12.0 mmol, 3.0 eq.) in Dioxane (20 mL) and water (S mL) was degassed and purged with N₂ for 3 times, and then the mixture was stirred at 80° C. for 12 hour under N₂ atmosphere. LCMS showed the reaction was completed. The reaction mixture was quenched by addition EA 10 mL at 20° C., and then diluted with water 10 mL and extracted with EA 60 mL (20 mL×3). The combined organic layers were washed with EA 15 mL (5 mL×3), dried over Na₂SO₄, filtered and concentrated under reduced pressure to give a residue. The crude material was purified on silica gel column chromatography (from pure PE to PE/EtOAc=4/1) to give 7-(2,6-bis(benzyloxy)pyridin-3-yl)-3,4-dihydronaphthalen-2 (1H)-one (300 mg, 689 μmol, 17.2%) as a yellow solid. LC-MS (ESI⁺) m/z: 436.1 (M+H)⁺.

Step 3: To a solution of 7-(2,6-bis(benzyloxy)pyridin-3-yl)-3,4-dihydronaphthalen-2 (1H)-one (300 mg, 689 μmol, 1.0 eq.) and tert-butyl piperazine-1-carboxylate (154 mg, 1.2 eq., 827 μmol) in MeOH (4 mL) and DCE (4 mL) was added Sodium triacetoxyborohydride (146 mg, 102 μL, 1 eq., 689 μmol). The mixture was stirred at 20° C. for 12 hour. LCMS showed the reaction was completed. The crude material was purified on silica gel column chromatography (from pure PE to PE/EtOAc=2/3) to give the product as brown oil.

LC-MS (ESI⁺) m/z: 606.4 (M+H)⁺.

¹H NMR (400 MHZ, CHLOROFORM-d) δ ppm 7.58 (d, J=8.11 Hz, 1H), 7.41-7.46 (m, 2H), 7.29-7.40 (m, 10H), 7.13 (d, J=7.99 Hz, 1H), 6.48 (d, J=8.11 Hz, 1H), 5.43 (s, 2H), 5.36 (s, 2H), 3.63-3.90 (m, 4H), 3.23-3.37 (m, 1H), 3.05-3.17 (m, 2H), 2.92-3.15 (m, 6H), 2.88-2.89 (m, 1H), 2.31-2.37 (m, 1H), 1.85 (m, 1H), 1.49 (s, 9H).

Step 4: The tert-butyl 4-(7-(2,6-bis(benzyloxy)pyridin-3-yl)-1,2,3,4-tetrahydronaphthalen-2-yl)piperazine-1-carboxylate (600 mg, 990 μmol, 1.0 eq.) was purified by SFC (Column: DAICEL CHIRALPAK AD (250 mm*30 mm, 10 um); Mobile phase: CO₂-EtOH (0.1% NH₃H₂O); from 50% to 50%; Flow rate: 80 mL/min) to give tert-butyl (R)-4-(7-(2,6-bis(benzyloxy)pyridin-3-yl)-1,2,3,4-tetrahydronaphthalen-2-yl)piperazine-1-carboxylate (220 mg, 363 μmol, 36.7%) and tert-butyl (S)-4-(7-(2,6-bis(benzyloxy)pyridin-3-yl)-1,2,3,4-tetrahydronaphthalen-2-yl)piperazine-1-carboxylate (210 mg, 347 μmol, 35.0%) both as white solid.

tert-butyl (R)-4-(7-(2,6-bis(benzyloxy)pyridin-3-yl)-1,2,3,4-tetrahydronaphthalen-2-yl)piperazine-1-carboxylate:

LC-MS (ESI⁺) m/z: 606.5 (M+H)⁺.

tert-butyl (S)-4-(7-(2,6-bis(benzyloxy)pyridin-3-yl)-1,2,3,4-tetrahydronaphthalen-2-yl)piperazine-1-carboxylate:

LC-MS (ESI⁺) m/z: 606.5 (M+H)⁺.

Step 5: To a solution of tert-butyl (S)-4-(7-(2,6-bis(benzyloxy)pyridin-3-yl)-1,2,3,4-tetrahydronaphthalen-2-yl)piperazine-1-carboxylate (200 mg, 330 μmol, 1.0 eq.) in EtOH (3 mL), DCM (3 mL) and EtOAc (3 mL) was added Pd/C (5 wt %, 0.05 g) under N₂ atmosphere. The suspension was degassed and purged with H₂ for 3 times. The mixture was stirred under H₂ (15 Psi) at 30° C. for 16 hour. LCMS showed the reaction was completed. The mixture was filtered and the filtrate was concentrated to give tert-butyl 4-((2S)-7-(2,6-dioxopiperidin-3-yl)-1,2,3,4-tetrahydronaphthalen-2-yl)piperazine-1-carboxylate (120 mg, 0.17 mmol, 51%, 60% Purity). LC-MS (ESI+) m/z; 428.2 (M+H)⁺.

To a solution of tert-butyl (R)-4-(7-(2,6-bis(benzyloxy)pyridin-3-yl)-1,2,3,4-tetrahydronaphthalen-2-yl)piperazine-1-carboxylate (200 mg, 330 μmol, 1.0 eq.) in EtOH (3 mL), DCM (3 mL) and EtOAc (3 mL) was added Pd/C (5%, 0.05 g) under N₂ atmosphere. The suspension was degassed and purged with H₂ for 3 times. The mixture was stirred under H₂ (15 Psi) at 30° C. for 16 hour. LCMS showed the reaction was completed. The mixture was filtered and the filtrate was concentrated to give tert-butyl 4-((2R)-7-(2,6-dioxopiperidin-3-yl)-1,2,3,4-tetrahydronaphthalen-2-yl)piperazine-1-carboxylate (120 mg, 0.17 mmol, 51%, 60% Purity) as a white solid. LC-MS (ESI+) m/z: 428.2 (M+H)⁺.

Step 6: To a solution of tert-butyl 4-((2S)-7-(2,6-dioxopiperidin-3-yl)-1,2,3,4-tetrahydronaphthalen-2-yl)piperazine-1-carboxylate (130 mg, 304 μmol, 1.0 eq.) in HCl/Dioxane (3 mL). The mixture was stirred at 20° C. for 2 hour. LCMS showed the reaction was completed. The mixture was concentrated in vacuum to give crude product. No further purification as it is used for the next step directly. LC-MS (ESI+) m/z: 328.0 (M+H)⁺.

To a solution of tert-butyl 4-((2R)-7-(2,6-dioxopiperidin-3-yl)-1,2,3,4-tetrahydronaphthalen-2-yl)piperazine-1-carboxylate (130 mg, 304 μmol, 1.0 eq.) in HCl/Dioxane (3 mL). The mixture was stirred at 20° C. for 2 hour. LCMS showed the reaction was completed. The mixture was concentrated in vacuum to give crude product. No further purification as it is used for the next step directly. LC-MS (ESI+) m/z: 328.0 (M+H)⁺.

Step 7: 3-((S)-7-(4-((1-(4-((1R,2S)-6-hydroxy-2-phenyl-1,2,3,4-tetrahydronaphthalen-1-yl)phenyl)piperidin-4-yl)methyl)piperazin-1-yl)-5,6,7,8-tetrahydronaphthalen-2-yl)piperidine-2,6-dione: To a solution of 1-(4-((1R,2S)-6-hydroxy-2-phenyl-1,2,3,4-tetrahydronaphthalen-1-yl)phenyl)piperidine-4-carbaldehyde (90 mg, 1.1 eq., 0.22 mmol) and 3-((S)-7-(piperazin-1-yl)-5,6,7,8-tetrahydronaphthalen-2-yl)piperidine-2,6-dione (65 mg, 1 eq., 0.20 mmol) in DCE (3 mL) and MeOH (3 mL) was added Sodium triacetoxyborohydride (0.13 g, 88 μL, 3 eq., 0.60 mmol). The mixture was stirred at 20° C. for 2 hour. LCMS showed the reaction was completed. The crude was purified by prep-HPLC together (C18-1 150*30 mm*5 um, water(NH₃H₂O+NH₄HCO₃)-ACN as a mobile phase, from 58% to 88%, Gradient Time (min): 11, Flow Rate (ml/min): 25) to give 3-((S)-7-(4-((1-(4-((1R,2S)-6-hydroxy-2-phenyl-1,2,3,4-tetrahydronaphthalen-1-yl)phenyl)piperidin-4-yl)methyl)piperazin-1-yl)-5,6,7,8-tetrahydronaphthalen-2-yl)piperidine-2,6-dione (36.7 mg, 50.8 μmol, 26%) as a white solid. LC-MS (ESI+) m/z: 723.4 (M+H)⁺.

LCMS: calc. for C₄₇H₅₄N₄O₃:722.42, found: [M+H]⁺723.4.

HPLC: 100.00% purity at 220 nm.

¹H NMR (400 MHZ, METHANOL-d₄) δ: ppm 7.04-7.12 (m, 4H), 6.95-7.00 (m, 2H), 6.78-6.82 (m, 2H), 6.69 (d, J=8.40 Hz, 1H), 6.60-6.65 (m, 3H), 6.51 (m, 1H), 6.30 (d, J=8.40 Hz, 2H), 4.18 (d, J=5.20 Hz, 1H), 3.78 (m, 1H), 3.51 (m, 2H), 2.85-3.07 (m, 5H), 2.69-2.84 (m, 7H), 2.61-2.68 (m, 2H), 2.49-2.60 (m, 5H), 2.16-2.28 (m, 6H), 1.83 (d, J=11.20 Hz, 2H), 1.73-1.79 (m, 1H), 1.59-1.69 (m, 2H), 1.26-1.35 (m, 3H).

### 3-((R)-7-(4-((1-(4-((1R,2S)-6-hydroxy-2-phenyl-1,2,3,4-tetrahydronaphthalen-1-yl)phenyl)piperidin-4-yl)methyl)piperazin-1-yl)-5,6,7,8-tetrahydronaphthalen-2-yl)piperidine-2,6-dione

To a solution of 1-(4-((1R,2S)-6-hydroxy-2-phenyl-1,2,3,4-tetrahydronaphthalen-1-yl)phenyl) piperidine-4-carbaldehyde (90 mg, 1.1 eq., 0.22 mmol) and 3-((R)-7-(piperazin-1-yl)-5,6,7,8-tetrahydronaphthalen-2-yl)piperidine-2,6-dione (65 mg, 1 eq., 0.20 mmol) in DCE (3 mL) and MeOH (3 mL) was added Sodium triacetoxyborohydride (0.13 g, 88 μL, 3 eq., 0.60 mmol). The mixture was stirred at 20° C. for 2 hour. LCMS showed the reaction was completed. The crude was purified by prep-HPLC together (C18-1 150*30 mm*5 um, water(NH₃H₂O+NH₄HCO₃)-ACN as a mobile phase, from 58% to 88%, Gradient Time (min): 11, Flow Rate (ml/min): 25) to give 3-((R)-7-(4-((1-(4-((1R,2S)-6-hydroxy-2-phenyl-1,2,3,4-tetrahydronaphthalen-1-yl)phenyl)piperidin-4-yl)methyl)piperazin-1-yl)-5,6,7,8-tetrahydronaphthalen-2-yl)piperidine-2,6-dione (22.4 mg, 31.0 μmol, 16%) as a white solid. LC-MS (ESI+) m/z: 723.3 (M+H)⁺.

LCMS: calc. for C₄₇H₅₄N₄O₃:722.42, found: [M+H]⁺723.3 HPLC: 93.69% purity at 220 nm.

¹H NMR (400 MHZ, METHANOL-d₄) δ: ppm 7.05-7.12 (m, 4H), 6.87-7.03 (m, 2H), 6.78-6.82 (m, 2H), 6.69 (d, J=8.40 Hz, 1H), 6.60-6.65 (m, 3H), 6.52 (m, 1H), 6.31 (d, J=8.40 Hz, 2H), 4.18 (d, J=4.80 Hz, 1H), 3.79 (d, J=4.00 Hz, 1H), 3.48-3.54 (m, 2H), 2.87-3.11 (m, 5H), 2.69-2.85 (m, 7H), 2.49-2.67 (m, 7H), 2.16-2.29 (m, 6H), 1.84 (d, J=12.40 Hz, 2H), 1.72-1.78 (m, 1H), 1.59-1.69 (m, 2H), 1.27-1.34 (m, 3H).

### Example 166. Preparation of (I-185) 3-(2-(1-((1-(4-((1S,2R)-6-hydroxy-2-phenyl-1,2,3,4-tetrahydronaphthalen-1-yl)phenyl)piperidin-4-yl)methyl)piperidin-4-yl)-1,2,3,4-tetrahydroisoquinolin-7-yl)piperidine-2,6-dione

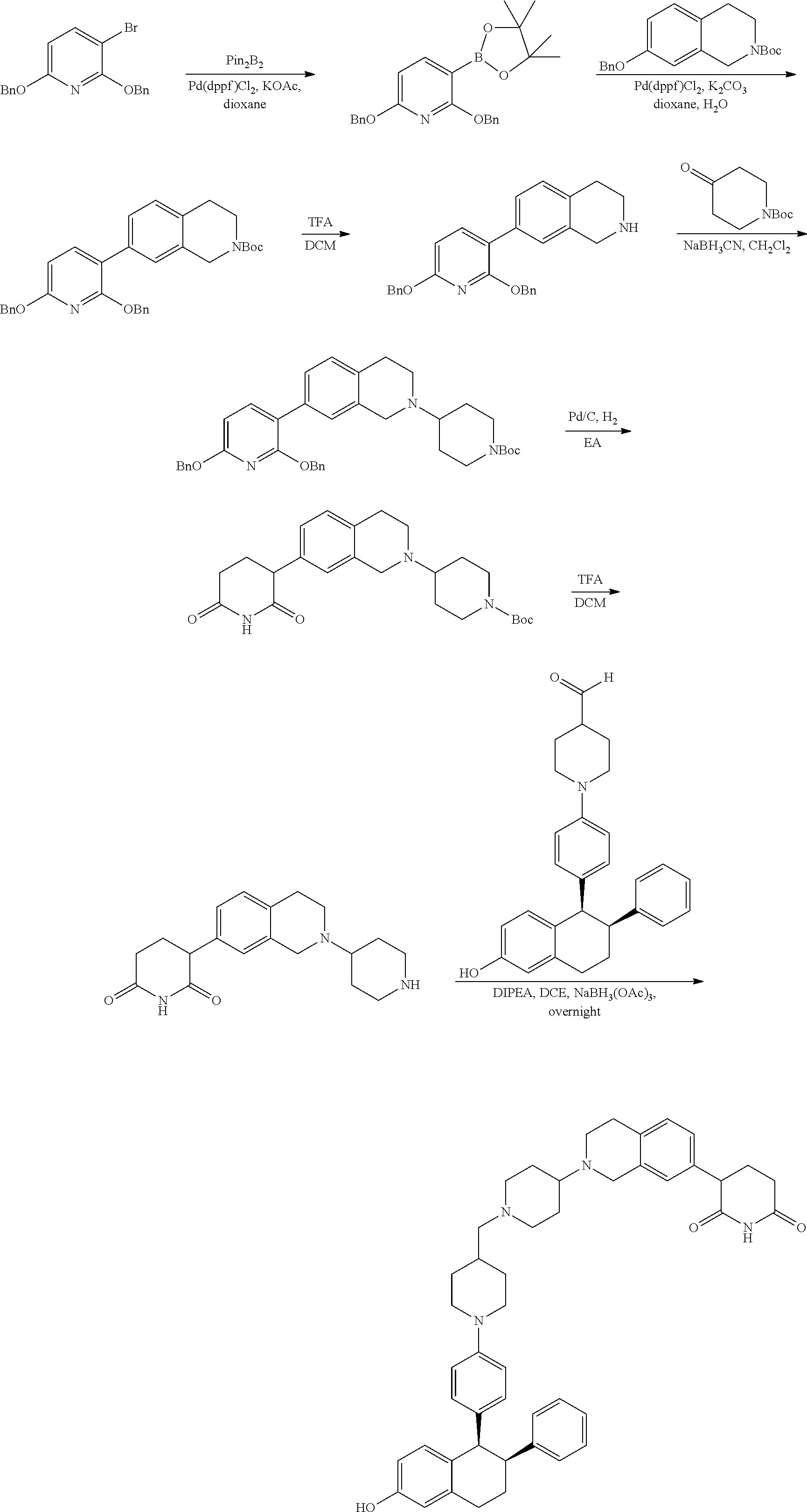

Step 1: A mixture of 2,6-bis(benzyloxy)-3-bromopyridine (3.0 g, 1 eq 8.10 mmol), Pin₂B₂ (2.26 g, 8.91 mmol, 1.1 eq.), Potassium acetate (1.59 g, 16.2 mmol, 2.0 eq.) and PdCl₂(dppf) (593 mg, 810 μmol, 0.1 eq.) in Dioxane (20 ml) was degassed and purged with N₂ for 3 times, and then the mixture was stirred at 90° C. for 6 hours under N₂ atmosphere. LCMS showed Reactant 1 was consumed completely and one main peak with desired MS was found. The reaction mixture was concentrated under reduced pressure to remove solvent. The residue was diluted with brine 20 mL and extracted with ethyl acetate 20 ml (20 ml×2). The combined organic layers were dried over Na₂SO₄, filtered and concentrated under reduced pressure to give a residue. The residue was purified by column chromatography (SiO₂, Petroleum ether/Ethyl acetate=5/1) to give compound 2 (1.25 g, 1.40 mmol, 17.3% yield, 46.724% Purity). LC-MS (ESI⁺) m/z: 418.2 (M+H)⁺.

Step 2: A mixture of 2,6-bis(benzyloxy)-3-(4,4,5,5-tetramethyl-1,3,2-dioxaborolan-2-yl)pyridine (900 mg, 2.16 mmol, 1.0 eq.), tert-butyl 7-bromo-3,4-dihydroisoquinoline-2 (1H)-carboxylate (741 mg, 2.37 mmol, 1.1 eq.), K₂CO₃ (596 mg, 4.31 mmol, 2.0 eq.), PdCl₂(dppf) (158 mg, 216 μmol, 0.1 eq.) in Dioxane (20 ml) and H₂O (0.2 ml) was degassed and purged with N₂ for 3 times, and then the mixture was stirred at 90° C. for 12 hours under N₂ atmosphere The reaction was clean according to LCMS showed reactant was consumed completely and one main peak with desired MS was detected The reaction mixture was concentrated under reduced pressure to remove solvent. The residue was diluted with brine 100 ml and extracted with ethyl acetate 100 ml (100 ml×2). The combined organic layers were dried with Na₂SO₄, filtered and concentrated under reduced pressure to give a residue The residue was purified by column chromatography (SiO₂, Petroleum ether/Ethyl acetate=7/1) to offer tert-butyl 7-(2,6-bis(benzyloxy)pyridin-3-yl)-3,4-dihydroisoquinoline-2 (1H)-carboxylate (550 mg, 1.02 mmol, 47.5%, 97.36% Purity). LC-MS (ESI⁺) m/z: 523.2 (M+H)⁺.

Step 3: To a solution of compound 4 (500 mg, 957 μmol, 1.0 eq.) in CH₂Cl₂ (10 ml) was added 2,2,2-trifluoroacetaldehyde (469 mg, 4.78 mmol, 5.0 eq.). The mixture was stirred at 25° C. for 6 hours. LC-MS showed 86% of desired compound was detected. The reaction mixture was quenched by addition NaHCO₃20 ml at 25° C., and then diluted with brine 220 ml and extracted with ethyl acetate 20 ml (20 ml×2). The combined organic layers were dried with Na₂SO₄, filtered and concentrated under reduced pressure to give a residue. The residue was purified by column chromatography (SiO₂, Petroleum ether/Ethyl acetate=5/1) to offer 7-(2,6-bis(benzyloxy)pyridin-3-yl)-1,2,3,4-tetrahydroisoquinoline (382 mg, 783 μmol, 81.8% yield, 86.58% Purity). LC-MS (ESI⁺) m/z: 423.2 (M+H)⁺.

Step 4: To a solution of 7-(2,6-bis(benzyloxy)pyridin-3-yl)-1,2,3,4-tetrahydroisoquinoline (400 mg, 947 μmol, 1.0 eq.) (The raw material contains trifluoroacetic acid) in CH₂Ch (10 ml) was added Triethylamine (192 mg, 264 μL, 2 eq, 1.89 mmol), Adjust the reaction solution to a weak base with triethylamine. The mixture was stirred at 20° C. for 10 min. And put tert-butyl 4-oxopiperidine-1-carboxylate (226 mg, 1.14 mmol, 1.2 eq.) into the reaction solution, Stir until completely dissolved in the reaction solution. Sodium triacetoxyborohydride (401 mg, 1.89 mmol, 2.0 eq.) was added into the reaction solution and stirred for 6 hours. LCMS showed Reactant 5 was consumed completely and one main peak with desired MS was detected. The reaction mixture was diluted with water 50 ml and extracted with CH₂Cl₂ 50 ml (20 ml×2). The combined organic layers were dried with Na₂SO₄, filtered and concentrated under reduced pressure to give a residue The residue was purified by column chromatography (SiO₂, Petroleum ether/Ethyl acetate=5/1) to offer tert-butyl 4-(7-(2,6-bis(benzyloxy)pyridin-3-yl)-3,4-dihydroisoquinolin-2 (1H)-yl)piperidine-1-carboxylate (100 mg, 142 μmol, 15.0% yield, 85.97% Purity). LC-MS (ESI+) m/z: 606.3 (M+H)⁺.

Step 5: A mixture of tert-butyl 4-(7-(2,6-bis(benzyloxy)pyridin-3-yl)-3,4-dihydroisoquinolin-2 (1H)-yl)piperidine-1-carboxylate (220 mg, 1 eq, 363 μmol) and Pd/C (19.3 mg, 182 μmol, 0.5 eq.) in MeOH (1 ml), ethyl acetate (1 ml) and CH₂Cl₂ (1 ml) was degassed and purged with H₂ for 3 times, and then the mixture was stirred at 30° C. for 12 hours under H₂ atmosphere. LCMS showed reactant 7 was consumed completely and no desired MS was detected. The catalyst and solid impurities of the reaction solution are filtered. The reaction mixture was concentrated under reduced pressure to remove solvent to offer tert-butyl 4-(7-(2,6-dioxopiperidin-3-yl)-3,4-dihydroisoquinolin-2 (1H)-yl)piperidine-1-carboxylate (122 mg, 80.2 μmol, 22.1% yield, 28.12% Purity). LC-MS (ESI⁺) m/z: 428.2 (M+H)⁺.

Step 6: To a solution of tert-butyl 4-(7-(2,6-dioxopiperidin-3-yl)-3,4-dihydroisoquinolin-2 (1H)-yl) piperidine-1-carboxylate (60 mg, 1 eq, 0.14 mmol) in CH₂Cl₂ (0.2 ml) was added TFA (0.16 g, 0.11 mL, 10 eq, 1.4 mmol). The mixture was stirred at 20° C. for 4 hours. LC-MS showed Reactant 8 was consumed completely and one main peak with desired MS was detected The reaction mixture was concentrated under reduced pressure to remove solvent to offer 3-(2-(piperidin-4-yl)-1,2,3,4-tetrahydroisoquinolin-7-yl)piperidine-2,6-dione (45 mg, 69 μmol, 49%, 50% Purity). LC-MS (ESI⁺) m/z: 328.2 (M+H).

Step 7: A mixture of 3-(2-(piperidin-4-yl)-1,2,3,4-tetrahydroisoquinolin-7-yl)piperidine-2,6-dione (70 mg, 0.17 mmol, 1.0 eq.) and 3-(2-(piperidin-4-yl)-1,2,3,4-tetrahydroisoquinolin-7-yl)piperidine-2,6-dione (56 mg, 0.17 mmol, 1.0 eq.) in CH₂Ch (1 ml) and MeOH (1 ml) was stirred for 5 minutes at 20° C., and then the mixture was added Diisopropylethylamine (44 mg, 0.34 mmol, 2.0 eq.) to adjust PH to about 6. Ten minutes later, Sodium triacetoxyborohydride (72 mg, 0.34 mmol, 2.0 eq.) was slowly added to the solution, stirred at 20° C. for 4 hour. LCMS showed reactant was remained. Several new peaks were shown on LC-MS and desired compound was detected. The reaction mixture was concentrated under reduced pressure to remove solvent. The residue was purified by prep-HPLC (neutral condition) to give desired compound 3-(2-(1-((1-(4-((1S,2R)-6-hydroxy-2-phenyl-1,2,3,4-tetrahydronaphthalen-1-yl)phenyl)piperidin-4-yl)methyl)piperidin-4-yl)-1,2,3,4-tetrahydroisoquinolin-7-yl)piperidine-2,6-dione (12.5 mg, 17.3 μmol, 10% yield, 100% Purity) as a white solid. LC-MS (ESI⁺) m/z: 723.6 (M+H)⁺.

LCMS: calc. for C₄₇H₅₄N₄O₃:722.97, found: [M+H]⁺723.6.

HPLC: 100% purity at 220 nm.

¹H NMR (400 MHz, METHANOL-d₄) δ ppm 7.07-7.16 (m, 4H), 6.95-7.05 (m, 2H), 6.76-6.83 (m, 2H), 6.69 (d, J=8.34 Hz, 1H), 6.58-6.66 (m, 3H), 6.49-6.54 (m, 1H), 6.26-6.34 (m, 2H), 4.15-4.21 (m, 1H), 3.76-3.83 (m, 3H), 3.50 (br d, J=10.37 Hz, 2H), 2.96-3.08 (m, 4H), 2.83-2.93 (m, 4H), 2.43-2.72 (m, 6H), 2.14-2.29 (m, 5H), 1.91-2.07 (m, 4H), 1.80-1.87 (m, 2H), 1.60-1.79 (m, 4H), 1.24-1.36 (m, 3H).

### Example 167. Preparation of (I-184) 3-(4-((1-((1-(4-((1R,2S)-6-hydroxy-2-phenyl-1,2,3,4-tetrahydronaphthalen-1-yl)phenyl)piperidin-4-yl)methyl)piperidin-4-yl)amino)phenyl)piperidine-2,6-dione

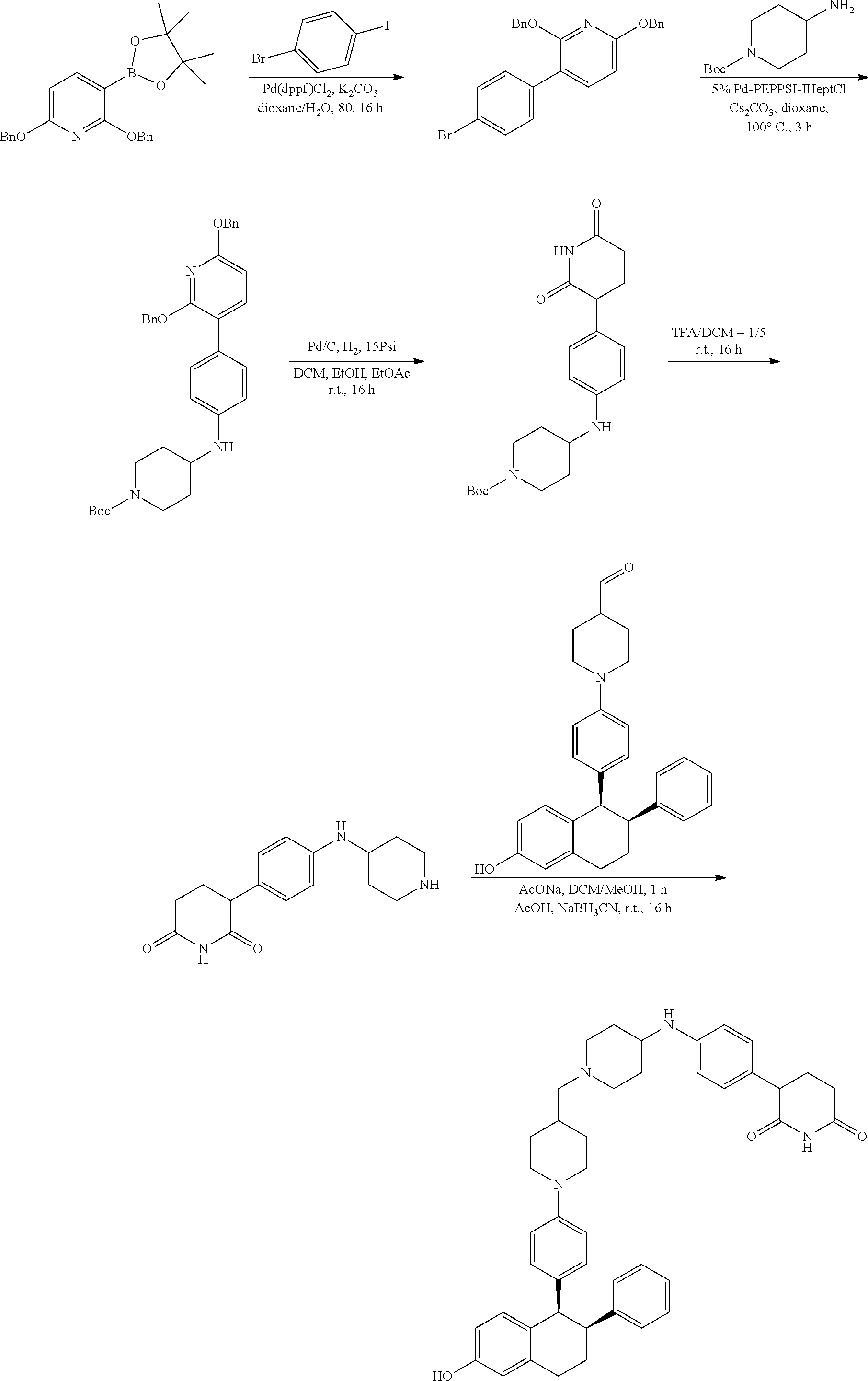

Step 1: A mixture of 1-bromo-4-iodobenzene (407 mg, 1.2 Eq, 1.44 mmol), 2,6-bis(benzyloxy)-3-(4,4,5,5-tetramethyl-1,3,2-dioxaborolan-2-yl)pyridine (500 mg, 1 Eq, 1.20 mmol), 1,1′-Bis(diphenylphosphino)ferrocene-palladium (II) dichloride (87.7 mg, 0.1 Eq, 120 μmol), potassium carbonate (331 mg, 140 μL, 2 Eq, 2.40 mmol) in 1,4-Dioxane (10 mL) and H₂O (2.0 mL) was degassed and purged with N₂ for 3 times, and then the mixture was stirred at 80° C. for 16 hour under N₂ atmosphere. TLC (petroleum ether:ethyl acetate=5:1, R_{f}=0.5 UV) showed one main new spot was observed. The reaction was quenched with water (50 mL) and extracted with ethyl acetate (150 mL*2). The organic layer was washed with brine (100 mL) and dried over Na₂SO₄ and concentrated in vacuo to give a white solid. The white solid was subjected to column chromatography over silica gel (gradient elution: 0-100% EtOAc). The desired fractions were collected, and concentrated to dryness in vacuo to give 2,6-bis(benzyloxy)-3-(4-bromophenyl)pyridine (340 mg, 762 μmol, 63.6%) as a white solid.

Step 2: A mixture of 1-T-Butoxycarbonyl-4-Aminopiperidine (169.2 mg, 1.3 Eq, 844.6 μmol), 2,6-bis(benzyloxy)-3-(4-bromophenyl)pyridine (290 mg, 1 Eq, 649.7 μmol), Pd-PEPPSI-IHeptCl (63.2 mg, 0.1 Eq, 65 μmol), cesium carbonate (635.1 mg, 3 Eq, 1.949 mmol) in dioxane (8 mL) was degassed and purged with N₂ for 3 times, and then the mixture was stirred at 100° C. for 12 bour under N₂ atmosphere. TLC (petroleum ether:ethyl acetate=5:1, R_{f}=0.5 UV) showed one main new spot was observed. The reaction was quenched with water (100 mL) and extracted with ethyl acetate (150 mL*2). The organic layer was washed with brine (100 mL) and dried over Na₂SO₄ and concentrated in vacuo to give a white solid. The white solid was subjected to column chromatography over silica gel (gradient elution: 0-100% EtOAc). The desired fractions were collected, and concentrated to dryness in vacuo to give tert-butyl 4-((4-(2,6-bis(benzyloxy)pyridin-3-yl)phenyl)amino)piperidine-1-carboxylate (280 mg, 495 μmol, 76.2%) as a white solid.

Step 3: A mixture of tert-butyl 4-((4-(2,6-bis(benzyloxy)pyridin-3-yl)phenyl)amino)piperidine-1-carboxylate (280 mg, 1 Eq, 495 μmol), Pd/C (280 mg, 10% Wt, 0.532 Eq, 263 μmol), hydrogen (1.00 mg, 1 Eq, 495 μmol) in DCM (10 mL), ethyl acetate (10 mL) and ethyl alcohol (10 mL) was degassed and purged with H₂ for 3 times, and then the mixture was stirred at 25° C. for 12 hour under H₂ atmosphere (15 psi). The reaction mixture was filtered and concentrated under reduced pressure to give a residue. The residue was concentrated under reduced pressure to give tert-butyl 4-((4-(2,6-dioxopiperidin-3-yl)phenyl)amino)piperidine-1-carboxylate (200 mg, 516 μmol, crude) as a yellow oil.

Step 4: To a mixture of tert-butyl 4-((4-(2,6-dioxopiperidin-3-yl)phenyl)amino)piperidine-1-carboxylate (200 mg, 1 Eq. 516 μmol) in DCM (5 mL) and TFA (1 mL), then the mixture was stirred at 25° C. for 16 hour. TLC (petroleum ether:ethyl acetate=5:1, R=0.5 UV) showed one main new spot was observed. The reaction mixture was filtered and concentrated under reduced pressure to give a residue. The residue was concentrated under reduced pressure to give 3-(4-(piperidin-4-ylamino)phenyl)piperidine-2,6-dione, as TFA salt (150 mg, 291 μmol, 56.4%) as a yellow oil.

Step 5: To a solution of 3-(4-(piperidin-4-ylamino)phenyl)piperidine-2,6-dione (150 mg, 1 Eq, 522 μmol), 1-(4-((1R,2S)-6-hydroxy-2-phenyl-1,2,3,4-tetrahydronaphthalen-1-yl)phenyl)piperidine-4-carbaldehyde (215 mg, 1 Eq, 522 μmol) in DCM (4 mL) and MeOH (4 mL) was added sodium acetate (257 mg, 6 Eq, 3.13 mmol) was stirred at 25° C. for 60 mins, Then NaBH CN (443 mg, 309 μL, 4 Eq, 2.09 mmol) and Acetic acid (188 mg, 180 μL, 6 Eq. 3.13 mmol) was added. The mixture was stirred at 25° C. for 16 hour. The reaction was quenched with H₂O (50 mL) and extracted with ethyl acetate (50 mL*2). The organic layer was washed with brine (30 mL) and dried over Na₂SO₄ and concentrated in vacuo to give a yellow oil. The residue was purified by prep-HPLC (column: Welch Xtimate C18 150*30 mm*5 um; mobile phase: [water (FA)-ACN]; B % 21%-51%, 7 min) to give 3-(4-((1-((1-(4-((1R,2S)-6-hydroxy-2-phenyl-1,2,3,4-tetrahydronaphthalen-1-yl)phenyl)piperidin-4-yl)methyl)piperidin-4-yl)amino)phenyl)piperidine-2,6-dione (62.5 mg, 85.0 μmol, 16.3%, 92.822% purity) was obtained as a white solid.

LCMS: calc. for C₄₄H₅₀N₄O₃:682.91, found: [M+H]⁺683.3.

HPLC: 92.822% purity at 220 nm.

¹H NMR (400 MHz, Methanol-da) 8=8.52 (br s, 0.737H), 7.11 (br d, J=1.3 Hz, 3H), 7.03 (d, J=8.6 Hz, 2H), 6.82 (dd, J=2.0, 7.2 Hz, 2H), 6.74-6.58 (m, 6H), 6.53 (dd, J=2.5, 8.2 Hz, 1H), 6.33 (d, J=8.6 Hz, 2H), 4.20 (br d, J=4.9 Hz, 1H), 3.73 (s, 1H), 3.57 (br d, J=10.0 Hz, 3H), 3.48 (br d, J=12.0 Hz, 2H), 3.35 (br s, 1H), 3.03 (br d, J=5.6 Hz, 4H), 2.95 (br d, J=6.6 Hz, 2H), 2.64 (br s, 4H), 2.30-2.12 (m, 5H), 1.94 (br d, J=3.2 Hz, 1H), 1.86 (brd, J=13.4 Hz, 2H), 1.82-1.64 (m, 3H), 1.49-1.31 (m, 2H).

SFC: retention time, 3.470/4.192 min; Area, 50.102%/49.898%;

### Example 168. Preparation of (I-183) 3-(3-(9-((1-(4-((1R,2S)-6-hydroxy-2-phenyl-1,2,3,4-tetrahydronaphthalen-1-yl)phenyl)piperidin-4-yl)methyl)-3,9-diazaspiro[5.5]undecan-3-yl)phenyl)piperidine-2,6-dione

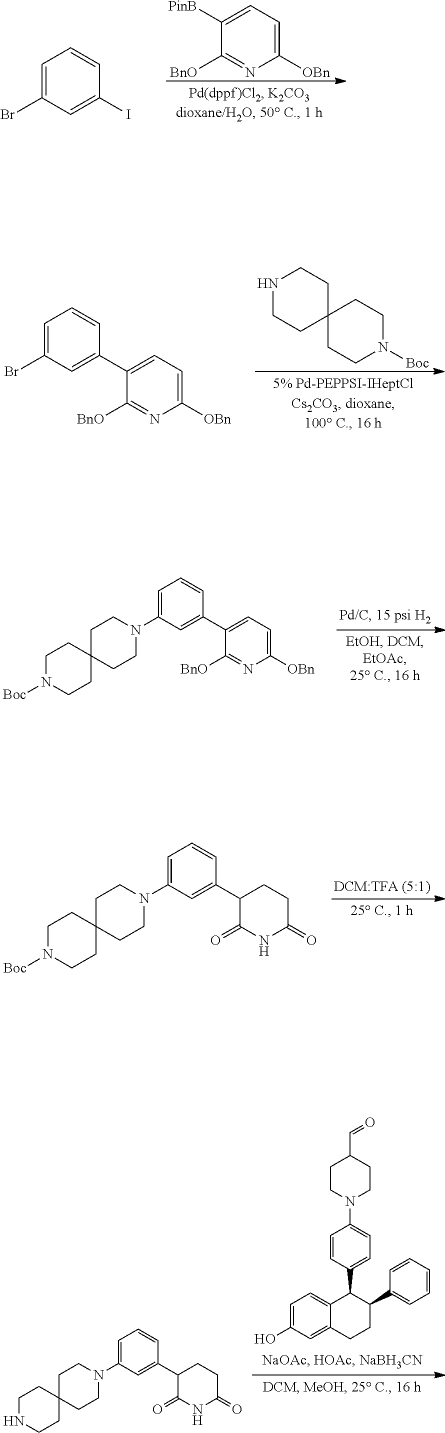

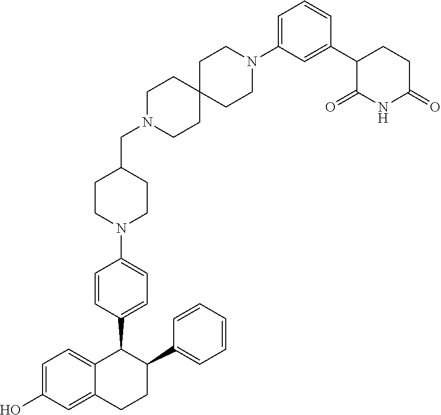 [SMILES: O=C1CCC(c2cccc(N3CCC4(CCN(CC5CCN(c6ccc([C@@H]7c8ccc(O)cc8CC[C@@H]7c7ccccc7)cc6)CC5)CC4)CC3)c2)C(=O)N1]

Step 1: To a stirred mixture of 2,6-bis(benzyloxy)-3-(4,4,5,5-tetramethyl-1,3,2-dioxaborolan-2-yl)pyridine (1 g, 1 Eq. 2 mmol) and 1-bromo-3-iodobenzene (0.8 g, 1.2 Eq, 3 mmol) in Dioxane (16 mL) and H₂O (4 mL) were added 1, 1′-Bis(di-t-butylphosphino)ferrocene palladium dichloride (0.2 g, 0.1 Eq, 0.2 mmol) and potassium carbonate (0.7 g, 2 Eq, 5 mmol) at room temperature under nitrogen atmosphere (3 times). The resulting mixture was stirred at 50° C. for 1 hour under nitrogen atmosphere. TLC (PE/EtOAc=10/1, R_{f}=0.5) showed two spots. The reaction mixture was quenched into a solution of saturated ammonium chloride solution followed by extraction with EtOAc (100 mL*2). The combined extracts was dried over anhydrous Na₂SO₄, filtered and concentrated to dryness in vacuo to give mixture as a brown oil. The brown oil was purified by flash silica gel chromatography (from PE/EtOAc=1/0 to 10/1). The desired fractions were collected, and concentrated to dryness in vacuo to give 2,6-bis(benzyloxy)-3-(3-bromophenyl)pyridine (439 mg, 301 μmol, 10%, 30.59% purity) as a colorless oil. LC-MS (ESI⁺) m/z: 448.0 (M+H)⁺.

Step 2: A mixture of tert-butyl 3,9-diazaspiro[5.5]undecane-3-carboxylate (65.0 mg, 1.1 Eq, 256 μmol), 2,6-bis(benzyloxy)-3-(3-bromophenyl)pyridine (339 mg, 30.59% Wt, 1 Eq, 232 μmol), Pd-PEPPSI-IHeptCl (22.6 mg, 0.1 Eq, 23.2 μmol) cesium carbonate (227 mg, 3 Eq, 697 μmol) in Dioxane (4 mL) was degassed and purged with N₂ for 3 times, and then the mixture was stirred at 100° C. for 16 hour under N₂ atmosphere. TLC (PE/EtOAc=10/1, R_{f}=0.5). The reaction mixture was quenched into a solution of saturated ammonium chloride solution followed by extraction with EtOAc (100 mL*2). The combined extracts was dried over anhydrous Na₂SO₄, filtered and concentrated to dryness in vacuo to give tert-butyl 9-(3-(2,6-bis(benzyloxy)pyridin-3-yl)phenyl)-3,9-diazaspiro[5.5]undecane-3-carboxylate as a green oil. The green oil was purified by flash silica gel chromatography (from PE/EtOAc=1/0 to 10/1). The desired fractions were collected, and concentrated to dryness in vacuo to give tert-butyl 9-(3-(2,6-bis(benzyloxy)pyridin-3-yl)phenyl)-3,9-diazaspiro[5.5]undecane-3-carboxylate (123 mg, 194 μmol, 83.5%, 97.79% Purity) as a green oil. LC-MS (ESI⁺) m/z; 620.2 (M+H)⁺.

Step 3: A mixture of tert-butyl 9-(3-(2,6-bis(benzyloxy)pyridin-3-yl)phenyl)-3,9-diazaspiro[5.5]undecane-3-carboxylate (123 mg, 97.791% Wt, 1 Eq, 194 μmol), dihydrogen (392 μg, 1 Eq, 194 μmol), Pd/C (124 mg, 10% Wt, 0.6 Eq) in DCM (2 mL), EtOAc (2 mL) and EtOH (2 mL) was degassed and purged with H₂ for 3 times, and then the mixture was stirred at 25° C. for 16 hour under H₂ atmosphere (15 psi). The reaction was filtered and concentrated to dryness in vacuo to give tert-butyl 9-(3-(2,6-dioxopiperidin-3-yl)phenyl)-3,9-diazaspiro[5.5]undecane-3-carboxylate (95 mg, 56 μmol, 29%, 26.07% purity) as a green oil. LC-MS (ESI+) m/z: 442.3 (M+H)⁺,

Step 4: A mixture of tert-butyl 9-(3-(2,6-dioxopiperidin-3-yl)phenyl)-3,9-diazaspiro[5.5]undecane-3-carboxylate (95 mg, 26.07% Wt, 1 Eq, 56 μmol) in DCM (2.5 mL) and TFA (0.5 mL), then the mixture was stirred at 25° C. for 1 hour. The reaction was concentrated to dryness in vacuo to give 3-(3-(3,9-diazaspiro[5.5]undecan-3-yl)phenyl)piperidine-2,6-dione (45 mg, 59 μmol, 45% purity) as a yellow oil. LC-MS (ESI+) m/z: 342.1 (M+H)⁺.

Step 5: A mixture of 1-(4-((1R,2S)-6-hydroxy-2-phenyl-1,2,3,4-tetrahydronaphthalen-1-yl)phenyl)piperidine-4-carbaldehyde (27 mg, 90% Wt, 1 Eq, 59 μmol), 3-(3-(3,9-diazaspiro[5.5]undecan-3-yl)phenyl)piperidine-2,6-dione (45 mg, 45% Wt, 1 Eq, 59 μmol) and sodium acetate (24 mg, 5 Eq, 0.30 mmol) in DCM (2 mL) and MeOH (2 mL) at 20° C. for 0.5 hour, pH=8, then acetic acid (11 mg, 10 μL, 3 Eq, 0.18 mmol) at 20° C. for 1 hour, pH=6, then added sodium cyanoborohydride (7.5 mg, 2 Eq, 0.12 mmol) the mixture, and then the mixture was stirred at 25° C. for 16 hour. The reaction was quenched with water (10 mL) and extracted with ethyl acetate (20 mL*2). The organic layer was washed with brine (10 mL) and dried over Na₂SO₄ and concentrated in vacuo to give 3-(3-(9-((1-(4-((1R,2S)-6-hydroxy-2-phenyl-1,2,3,4-tetrahydronaphthalen-1-yl)phenyl)piperidin-4-yl)methyl)-3,9-diazaspiro[5.5]undecan-3-yl)phenyl)piperidine-2,6-dione as a brown oil. The brown oil was purified by preparative high-performance liquid chromatography. Condition: Column: Xtimate C18 150*40 mm*10 um, A: water (FA), B: CAN, at the beginning: A (75%) and B (25%), at the end: A: (45%) and B (55%), Gradient Time (min) 6; 100% B hold Time (min) 2, Flow Rate (ml/min) 25. The pure fractions were collected and the solvent was evaporated under vacuum. The aqueous layer was lyophilized to dryness to give 3-(3-(9-((1-(4-((1R,2S)-6-hydroxy-2-phenyl-1,2,3,4-tetrahydronaphthalen-1-yl)phenyl)piperidin-4-yl)methyl)-3,9-diazaspiro[5.5]undecan-3-yl)phenyl)piperidine-2,6-dione (38.3 mg, 50.9 μmol, 86%, 97.99% purity) as a white solid.

LCMS: calc. for C₄₈H₅₆N₄O₃: 736.44, found: [M+H]⁺737.5.

HPLC: 97.99% purity at 220 nm.

¹H NMR (400 MHZ, DMSO-d₆) δ=10.80 (s, 1H), 9.11 (br s, 1H), 8.15 (s, 0.731H), 7.18-7.07 (m, 4H), 6.86-6.75 (m, 4H), 6.66-6.44 (m, 6H), 6.19 (d, J=8.7 Hz, 2H), 4.12 (d, J=4.8 Hz, 1H), 3.74 (dd, J=4.9, 11.1 Hz, 1H), 3.54-3.45 (m, 3H), 3.27 (br s, 2H), 3.10 (br s, 4H), 3.01-2.87 (m, 2H), 2.61 (br d, J=11.8 Hz, 1H), 2.48-2.42 (m, 3H), 2.38 (br s, 3H), 2.24-1.97 (m, 5H), 1.70 (br d, J=11.9 Hz, 3H), 1.59 (br s, 1H), 1.50 (br d, J=19.0 Hz, 7H), 1.11 (br dd, J=1.8, 11.2 Hz, 2H)

SFC: retention time, 2.028 min, 3.493 min; Area, 50.13%, 49.87%;

### Example 169. Preparation of (I-182) 3-(4-(2-((1-(4-((1R,2S)-6-hydroxy-2-phenyl-1,2,3,4-tetrahydronaphthalen-1-yl)phenyl)piperidin-4-yl)methyl)-2-azaspiro[3.3]heptan-6-yl)phenyl)piperidine-2,6-dione

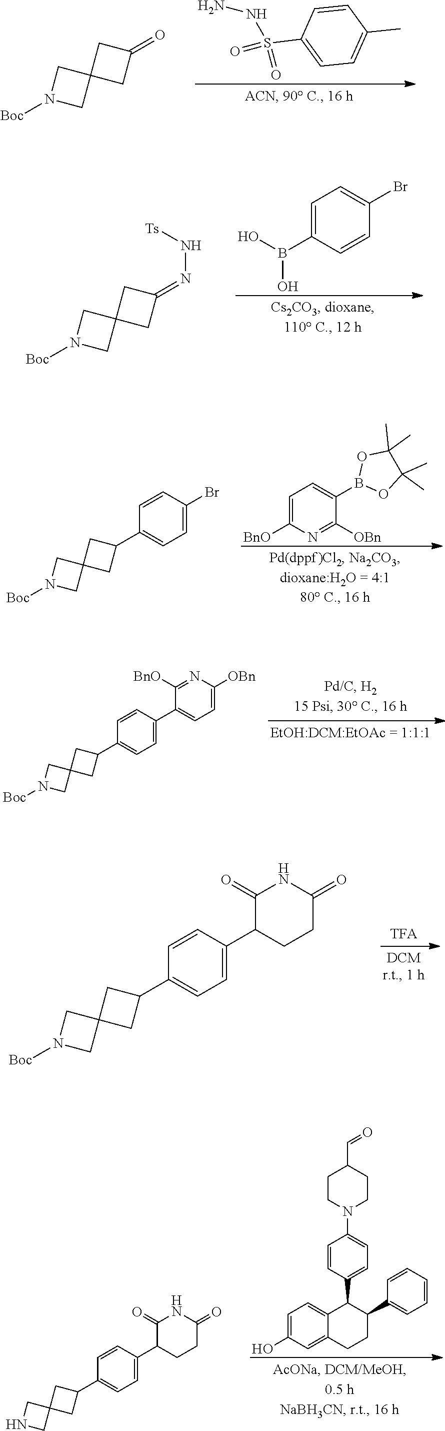

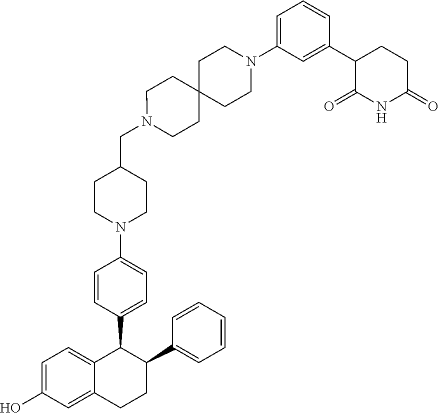 [SMILES: O=C1CCC(c2cccc(N3CCC4(CCN(CC5CCN(c6ccc([C@@H]7c8ccc(O)cc8CC[C@@H]7c7ccccc7)cc6)CC5)CC4)CC3)c2)C(=O)N1]

Step 1: A mixture of tert-butyl 6-oxo-2-azaspiro[3.3]heptane-2-carboxylate (2 g, 9.47 mmol, 1 eq), 4-methylbenzenesulfonohydrazide (2.12 g, 11.36 mmol, 1.2 eq) was added in the ACN (20 mL), the mixture was stirred at 90° C. for 16 hr. Then the mixture was treated with H₂O (100 mL), extracted with EtOAc (150 mL). The combined extracts was dried over anhydrous Na₂SO₄, filtered and concentrated to dryness in vacuo to give a residue. The residue was subjected to column chromatography over silica gel (gradient elution: 0˜10% ethyl acetate in petroleum ether). The pure fractions were collected and concentrated to dryness in vacuo to give tert-butyl 6-(2-tosylhydrazineylidene)-2-azaspiro[3.3]heptane-2-carboxylate (1.67 g, 4.40 mmol, 46.49% yield) as a yellow oil. LC-MS (ESI+) m/z: 380 (M+H)⁺.

Step 2: A mixture of tert-butyl 6-(2-tosylhydrazineylidene)-2-azaspiro[3.3]heptane-2-carboxylate (1 g, 2.64 mmol, 1 eq), (4-bromophenyl)boronic acid (740.9 mg, 3.7 mmol, 1.4 eq), Cs₂CO₃ (1.7 g, 5.3 mmol, 2 eq) was added in dioxane (10 mL), the mixture was stirred at 110° C. for 12 hr. Then the mixture was treated with H₂O (50 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜30% ethyl acetate in petroleum ether). The pure fractions were collected and concentrated to dryness in vacuo to give tert-butyl 6-(4-bromophenyl)-2-azaspiro[3.3]heptane-2-carboxylate (330 mg, 936.8 μmol, 35.6% yield) as a yellow oil. LC-MS (ESI+) m/z: 352 (M+H)⁺.

Step 3: A mixture of tert-butyl 6-(4-bromophenyl)-2-azaspiro[3.3]heptane-2-carboxylate (300 mg, 851.63 μmol, 1 eq), 2,6-bis(benzyloxy)-3-(4,4,5,5-tetramethyl-1,3,2-dioxaborolan-2-yl)pyridine (426.47 mg, 1.02 mmol, 1.2 eq), disodium carbonate (270.8 mg, 2.55 mmol, 3 eq), Pd(dppf) C12 (69.55 mg, 85.16 μmol, 0.1 eq) in dioxane (4 mL) and H₂O (1 mL) was degassed and purged with N₂ for 3 times, and then the mixture was stirred at 80° C. for 16 hr under N₂ atmosphere. Then the mixture was treated with H₂O (50 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜50% ethyl acetate in petroleum ether). The pure fractions were collected and concentrated to dryness in vacuo to give tert-butyl 6-(4-(2,6-bis(benzyloxy)pyridin-3-yl)phenyl)-2-azaspiro[3.3]heptane-2-carboxylate (450 mg, 799.72 μmol, 93.90% yield) was obtained as a white solid. LC-MS (ESI+) m/z: 563.2 (M+H)⁺.

Step 4: To a solution of tert-butyl 6-(4-(2,6-bis(benzyloxy)pyridin-3-yl)phenyl)-2-azaspiro[3.3]heptane-2-carboxylate (350 mg, 622 μmol, 1 eq) and Pd/C (662 mg, 622.01 μmol, 10% purity, 1 eq) in DCM (2 mL), EtOAc (2 mL) and EtOH (2 mL) and purged with H₂ for 3 times, the mixture was stirred at 25° C. for 12 hr. The reaction mixture was filtered and concentrated to dryness in vacuo to give tert-butyl 6-(4-(2,6-dioxopiperidin-3-yl)phenyl)-2-azaspiro[3.3]heptane-2-carboxylate (230 mg, 598.23 μmol, 96.18% yield). LC-MS (ESI⁺) m/z: 385 (M+H)⁺.

Step 5: A mixture of tert-butyl 6-(4-(2,6-dioxopiperidin-3-yl)phenyl)-2-azaspiro[3.3]heptane-2-carboxylate (150 mg, 390.2 μmol, 1 eq) was added in TFA (0.2 mL) and DCM (2 mL), the mixture was stirred at 25° C. for 1 hr. LCMS showed 95% of desired compound was detected. The reaction mixture was filtered and concentrated to dryness in vacuo to give 3-(4-(2-azaspiro[3.3]heptan-6-yl)phenyl)piperidine-2,6-dione (96 mg, 337.6 μmol, 86.53% yield). LC-MS (ESI⁺) m/z: 285 (M+H)⁺.

Step 6: A mixture of 3-(4-(2-azaspiro[3.3]heptan-6-yl)phenyl)piperidine-2,6-dione (96 mg, 337.61 μmol, 1 eq), 1-(4-((1R,2S)-6-hydroxy-2-phenyl-1,2,3,4-tetrahydronaphthalen-1-yl)phenyl)piperidine-4-carbaldehyde (139 mg, 337.6 μmol, 1 eq), sodium acetate (166.2 mg, 2 mmol, 6 eq) was added in DCM (4 mL) and MeOH (4 mL), the mixture was stirred at 25° C. for 0.5 h, and then sodium; triacetoxyboranuide (286.2 mg, 1.35 mmol, 4 eq), acetic acid (559.3 mg, 2 mmol, 532.7 μL, 6 eq) was added in the mixture, the mixture was stirred at 25° C. for 16 hr. LCMS showed 49% of desired compound was detected. The reaction mixture was treated with H₂O 30 mL and extracted with EtOAc 50 mL (50 mL*2). The organic layers were dried with anhydrous Na₂SO₄, filtered, the residue was purified by prep. HPLC (column: Welch Xtimate C18 150*30 mm*5 um; mobile phase: [water (FA)-ACN]; gradient: 24%-54% B over 7 min). The aqueous phase was lyophilized to dryness to give 3-(4-(2-((1-(4-((1R,2S)-6-hydroxy-2-phenyl-1,2,3,4-tetrahydronaphthalen-1-yl)phenyl)piperidin-4-yl)methyl)-2-azaspiro[3.3]heptan-6-yl)phenyl)piperidine-2,6-dione (29 mg, 42.65 μmol, 12.63% yield) as a yellow solid.

LC-MS (ESI+) m/z: 679 (M+H)⁺.

HPLC: 97.341%, purity at 220 nm.

SFC: retention time, 2.360, 3.019 min; Area, 49.908%, 50.092%;

¹H NMR (400 MHZ, DMSO-d₆) δ=10.85-10.76 (m, 1H), 8.17 (s, 0.707H), 7.26-7.06 (m, 7H), 6.86-6.77 (m, 2H), 6.67-6.56 (m, 2H), 6.55-6.43 (m, 3H), 6.22-6.13 (m, 2H), 4.17-4.06 (m, 1H), 3.84-3.75 (m, 1H), 3.53-3.41 (m, 6H), 3.37-3.32 (m, 3H), 3.10-2.83 (m, 3H), 2.71-2.53 (m, 2H), 2.45-2.39 (m, 4H), 2.22-1.91 (m, 5H), 1.67 (br d, J=12.2 Hz, 3H), 1.45-1.30 (m, 1H), 1.21-1.05 (m, 2H).

### Example 170. Preparation of (I-421) 3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione

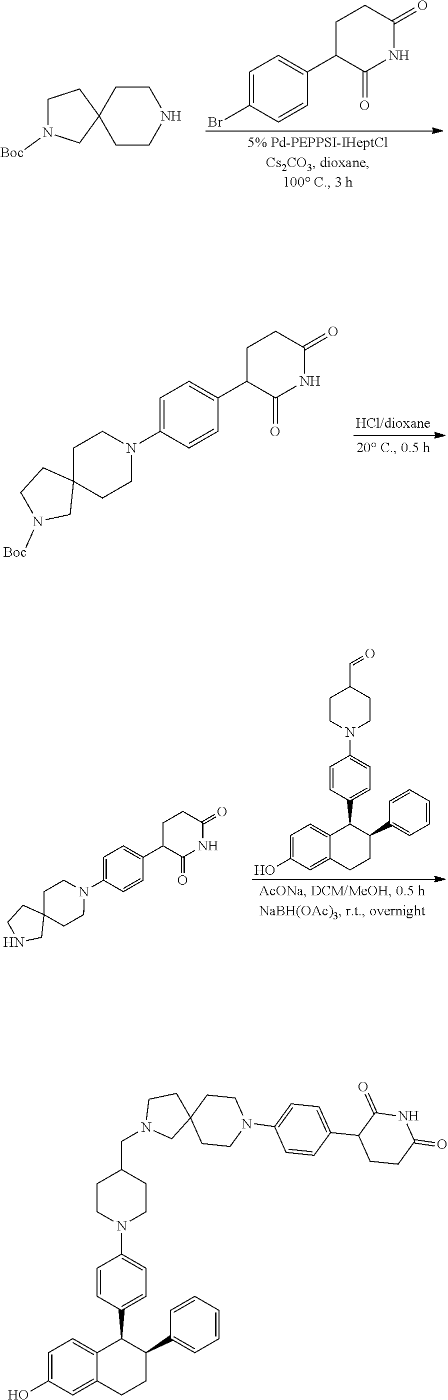 [SMILES: CC(C)(C)OC(=O)N1CCC2(CCNCC2)C1 ; O=C1CCC(c2ccc(Br)cc2)C(=O)N1 ; CC(C)(C)OC(=O)N1CCC2(CCN(c3ccc(C4CCC(=O)NC4=O)cc3)CC2)C1 ; O=C1CCC(c2ccc(N3CCC4(CCNC4)CC3)cc2)C(=O)N1 ; O=CC1CCN(c2ccc([C@@H]3c4ccc(O)cc4CC[C@@H]3c3ccccc3)cc2)CC1 ; O=C1CCC(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1]

Step 1: A mixture of 3-(4-bromophenyl)piperidine-2,6-dione (150 mg, 559.5 μmol, 1 eq), tert-butyl 2,8-diazaspiro[4.5]decane-2-carboxylate (174.8 mg, 727.3 μmol, 1.3 eq), dicesium; carbonate (546.9 mg, 1.68 mmol, 3 eq), Pd-PEPPSI-IHeptCl (54.4 mg, 56 μmol, 0.1 eq) in dioxane (5 mL) was degassed and purged with N₂ for 3 times, and then the mixture was stirred at 100° C. for 3.5 hr under N₂ atmosphere. The reaction mixture was quenched by addition 50 mL H₂O at 20° C., and then diluted with 50 mL ethyl acetate and extracted with ethyl acetate (50 mL*3). The combined organic layers were washed with 50 mL brine, dried over drying Na₂SO₄, filtered and concentrated under reduced pressure to give a residue. The residue was purified by flash silica gel chromatography (ISCO®; 4 g SepaFlash®Silica Flash Column, Eluent of 0˜20% DCM/MeOH @ 30 mL/min). Compound tert-butyl-8-(4-(2,6-dioxopiperidin-3-yl)phenyl)-2,8-diazaspiro[4.5]decane-2-carboxylate (137 mg, 320.44 μmol, 57.27% yield) was obtained as a brown oil.

LC-MS (ESI⁺) m/z: 428.2 (M+H)⁺.

Step 2: To a solution of tert-butyl 8-[4-(2,6-dioxo-3-piperidyl)phenyl]-2,8-diazaspiro[4.5]decane-2-carboxylate (137 mg, 320.4 μmol, 1 eq) in HCl/dioxane (8 mL). The mixture was stirred at 20° C. for 0.5 hr. TLC indicated no start material was remained, and one major new spot with larger polarity was detected (PE:EA=0:1 R_{f}=0.1). The reaction mixture was concentrated under reduced pressure to remove HCl/dioxane and give a white solid. The crude product 3-[4-(2,8-diazaspiro[4.5]decan-8-yl)phenyl]piperidine-2,6-dione (166 mg, crude) as white solid was used into the next step without further purification.

Step 3: A mixture of 3-[4-(2,8-diazaspiro[4.5]decan-8-yl)phenyl]piperidine-2,6-dione (166 mg, 507 μmol, 1 eq), 1-(4-((1R,2S)-6-hydroxy-2-phenyl-1,2,3,4-tetrahydronaphthalen-1-yl)phenyl)piperidine-4-carbaldehyde (313 mg, 760.5 μmol, 1.5 eq) and sodium; acetate (249.5 mg, 3 mmol, 6 eq) and acetic acid (121.8 mg, 2 mmol, 116.1 μL, 4 eq) in DCM (2 mL) and MeOH (2 mL) at 30° C. for 1 hour, then NaBH(OAc)₃ (429.81 mg, 2.03 mmol, 4 eq) was added to the mixture and was stirred at 30° C. for 16 hr The reaction mixture was quenched by addition H₂O 20 mL at 25° C., and then diluted with 20 mL ethyl acetate and extracted with ethyl acetate (20 mL*3). The combined organic layers were washed with brine 20 mL, dried over anhydrous Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 17%-47% B over 7 min) Compound 3-(4-(2-((1-(4-((1R,2S)-6-hydroxy-2-phenyl-1,2,3,4-tetrahydronaphthalen-1-yl)phenyl)piperidin-4-yl)methyl)-2,8-diazaspiro[4.5]decan-8-yl)phenyl)piperidine-2,6-dione (16.4 mg, 21.72 μmol, 4.28% yield, 95.744% purity) was obtained as a white solid.

LC-MS (ESI⁺) m/z: 723.4 (M+H)⁺.

HPLC: 95.744% purity at 220 nm.

¹H NMR (400 MHz, DMSO-d₆) δ=12.70-12.25 (m, 1H), 7.94 (s, 1H), 7.16 (br d, J=1.8 Hz, 3H), 7.11 (d, J=8.5 Hz, 2H), 6.93 (d, J=8.8 Hz, 2H), 6.82 (s, 3H), 6.73 (d, J=2.3 Hz, 1H), 6.64-6.59 (m, 1H), 6.56 (d, J=8.5 Hz, 2H), 6.30 (d, J=8.5 Hz, 2H), 4.21 (d, J=4.8 Hz, 1H), 3.74 (dd, J=5.1, 9.4 Hz, 1H), 3.65-3.52 (m, 2H), 3.40-3.32 (m, 1H), 3.24-3.14 (m, 4H), 3.05 (br s, 5H), 2.62 (br s, 5H), 2.25 (s, 3H), 2.11 (s, 6H), 1.87-1.75 (m, 4H), 1.53-1.39 (m, 4H)

SFC: retention time, 3.550/4.818 min; Area, 50.274/49.726%;

### Example 171. Preparation of (I-180) 3-[4-[9-[[1-[4-[(1R,2S)-6-hydroxy-2-phenyl-tetralin-1-yl]phenyl]-4-piperidyl]methyl]-3,9-diazaspiro[5.5]undecan-3-yl]phenyl]piperidine-2,6-dione

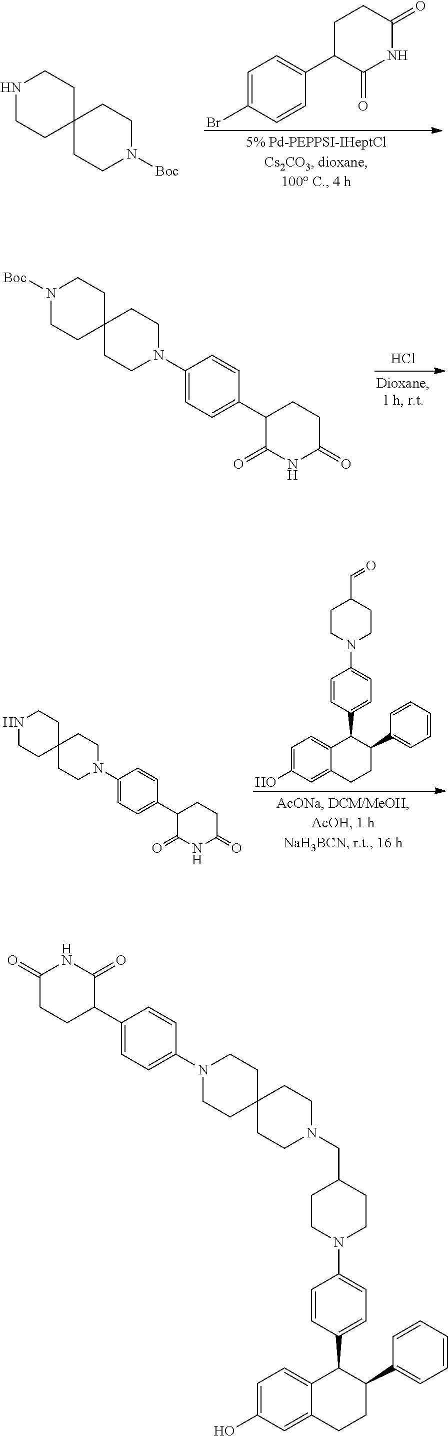

Step 1: A mixture of tert-butyl 3,9-diazaspiro[5.5]undecane-3-carboxylate (113.9 mg, 447.6 μmol, 1.2 eq), 3-(4-bromophenyl)piperidine-2,6-dione (100 mg, 373 μmol, 1 eq), Cs₂CO₃ (364.6 mg, 1.12 mmol, 3 eq), Pd-PEPPSI-IHeptCl (36.3 mg, 37.3 μmol, 0.1 eq) in dioxane (4 mL) was degassed and purged with N₂ for 3 times, and then the mixture was stirred at 100° C. for 4 hr under N₂ atmosphere. TLC (DCM/MeOH=10:1, R_{f}=0.4) showed new spot was formed. The reaction mixture was diluted with 10 mL H₂O and extracted with 20 mL EA (10 mL*2). The combined organic layers were washed with brine (5 mL*2), dried over Na₂SO₄, filtered and concentrated under reduced pressure to give a residue. The residue was purified by column chromatography (SiO₂, DCM:MeOH=10:1). Compound tert-butyl 9-[4-(2,6-dioxo-3-piperidyl)phenyl]-3,9-diazaspiro[5.5]undecane-3-carboxylate (100 mg, 226.5 μmol, 60.7% yield) was obtained as a yellow solid. LC-MS (ESI+) m/z: 442.3 (M+H)⁺.

Step 2: To a solution of tert-butyl 9-[4-(2,6-dioxo-3-piperidyl)phenyl]-3,9-diazaspiro[5.5]undecane-3-carboxylate (100 mg, 226.47 μmol, 1 eq) in 4 M HCl/dioxane (8 mL) was stirred at 25° C. for 1 hr. TLC (petroleum ether/ethyl acetate=5:1, R_{f}=0.4) showed new spot was formed. The reaction mixture was concentrated under reduced pressure to give a residue. The crude product 3-[4-(3,9-diazaspiro[5.5]undecan-3-yl)phenyl]piperidine-2,6-dione (70 mg, 205.01 μmol, 90.52% yield) was used into the next step without further purification. Compound 3-[4-(3,9-diazaspiro[5.5]undecan-3-yl)phenyl]piperidine-2,6-dione (70 mg, 205.01 μmol, 90.52% yield) was obtained as a yellow solid. LC-MS (ESI+) m/z: 342.2 (M+H)⁺.

Step 3: To a solution of 1-[4-[(1R,2S)-6-hydroxy-2-phenyl-tetralin-1-yl]phenyl]piperidine-4-carbaldehyde (67.50 mg, 164.01 μmol, 1 eq) and 3-[4-(3,9-diazaspiro[5.5]undecan-3-yl)phenyl]piperidine-2,6-dione (70 mg, 164.01 μmol, 1 eq, 2HCL) in DCM (4 mL) and MeOH (4 mL) was added sodium acetate (80.73 mg, 984.05 μmol, 6 eq). The mixture was stirred at 30° C. for 1 hr. Then the mixture was added acetic acid (59.09 mg, 984.05 μmol, 56.33 μL, 6 eq), NaH₃BCN, (41.23 mg, 656.03 μmol, 4 eq). The mixture was stirred at 30° C. for 12 hr finally. The reaction mixture was diluted with 10 mL H₂O and extracted with DCM (10 mL*2). The combined organic layers were washed with brine 10 mL (5 mL*2), dried over Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 18%-48% B over 7 min). Then the residue was further purified by prep-HPLC (column: Welch Xtimate C18 150*30 mm*5 um; mobile phase: [water (FA)-ACN]; gradient: 21%-41% B over 7 min). Compound 3-[4-[9-[1-[4-[(1R,2S)-6-hydroxy-2-phenyl-tetralin-1-yl]phenyl]-4-piperidyl]methyl]-3,9-diazaspiro[5.5]undecan-3-yl]phenyl]piperidine-2,6-dione (10.8 mg, 14.65 μmol, 8.94% yield) was obtained as a white solid.

LC-MS (ESI⁺) m/z: 737.5 (M+H)⁺.

HPLC: 97.978%, purity at 220 nm.

b: Rt: 3.999 min; Area, 50.204%, Rt: 7.448 min; Area, 49.796%;

¹H NMR (400 MHZ, METHANOL-da) δ ppm 8.52-8.60 (m, 0.641H) 7.09-7.18 (m, 5H) 7.00 (d. J=8.70 Hz, 2H) 6.79-6.86 (m, 2H) 6.70 (d, J=8.46 Hz, 1H) 6.60-6.67 (m, 3H) 6.50-6.56 (m, 1H) 6.29-6.37 (m, 2H) 4.20 (br d, J=5.25 Hz, 1H) 3.76-3.83 (m, 1H) 3.52-3.60 (m, 2H) 3.31-3.33 (m, 4H) 3.15-3.23 (m, 4H) 3.07 (br s, 1H) 2.96-3.05 (m, 4H) 2.80 (br d, J=4.41 Hz, 1H) 2.56-2.71 (m, 4H) 2.16-2.29 (m, 3H) 1.85 (br d, J=11.92 Hz, 3H) 1.75 (br d, J=16.33 Hz, 8H) 1.33-1.46 (m, 2H).

### Example 172. Preparation of (I-179) 3-(3-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione

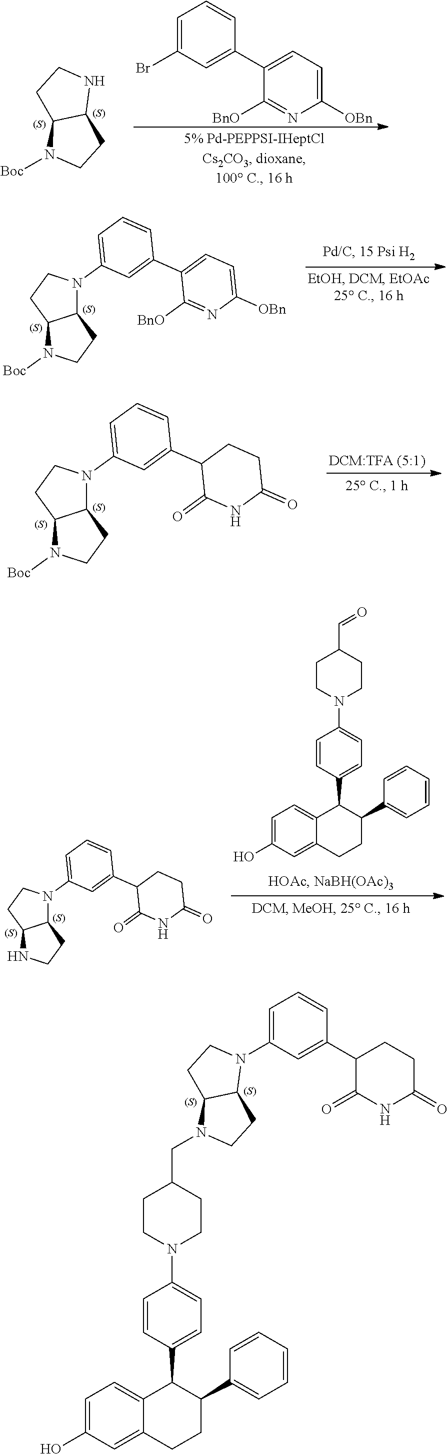

Step 1: A mixture of tert-butyl (3aS,6aS)-hexahydropyrrolo[3,2-b]pyrrole-1 (2H)-carboxylate (20.0 mg, 1 Eq, 94.2 μmol), 2,6-bis(benzyloxy)-3-(3-bromophenyl)pyridine (46.7 mg, 1.1 Eq, 104 μmol), Pd-PEPPSI-IHeptCI (4.58 mg, 0.05 Eq, 4.71 μmol) and cesium carbonate (92.1 mg, 3 Eq, 283 μmol) added in Dioxane (1 mL) was degassed and purged with N₂ for 3 times, and then the mixture was stirred at 85° C. for 16 hour under N₂ atmosphere. TLC (PE/EtOAc=10/1, R_{f}=0.4) showed one new spot. The reaction mixture was quenched into a solution of saturated ammonium chloride (20 mL) solution followed by extraction with EtOAc (50 mL*3). The combined extracts was dried over anhydrous Na₂SO₄, filtered and concentrated to dryness in vacuo to give mixture as a brown oil. The brown oil was purified by flash silica gel chromatography (from PE/EtOAc=1/0 to 10/1). The desired fractions were collected, and concentrated to dryness in vacuo to give tert-butyl (3aS,6aS)-4-(3-(2,6-bis(benzyloxy)pyridin-3-yl)phenyl) hexahydropyrrolo[3,2-b]pyrrole-1 (2H)-carboxylate (50 mg, 84 μmol, 89%, 97% purity) as a colorless oil. LC-MS (ESI⁺) m/z: 578.1 (M+H)⁺.

Step 2: A mixture of tert-butyl (3aS,6aS)-4-(3-(2,6-bis(benzyloxy)pyridin-3-yl)phenyl) hexahydropyrrolo[3,2-b]pyrrole-1 (2H)-carboxylate (50 mg, 1 Eq, 84 μmol), dihydrogen (0.17 mg, 1 Eq, 84 μmol), palladium on carbon (25 mg, 10% Wt, 0.28 Eq, 23 μmol) in DCM (2 mL), EtOAc (2 mL) and EtOH (2 mL) was degassed and purged with H₂ for 3 times, and then the mixture was stirred at 25° C. for 16 hour under H₂ atmosphere (15 psi). The reaction was filtered and concentrated to dryness in vacuo to give tert-butyl (3aS,6aS)-4-(3-(2,6-dioxopiperidin-3-yl)phenyl) hexahydropyrrolo[3,2-b]pyrrole-1 (2H)-carboxylate (49 mg, 58 μmol, 69%, 47% purity) as a colorless oil. LC-MS (ESI⁺) m/z: 399.9 (M+H)⁺.

Step 3: A mixture of tert-butyl (3aS,6aS)-4-(3-(2,6-dioxopiperidin-3-yl)phenyl) hexahydropyrrolo[3,2-b]pyrrole-1 (2H)-carboxylate (49 mg, 47% Wt, 1 Eq, 58 μmol) in DCM (2.5 mL) and TFA (0.5 mL), then the mixture was stirred at 25° C. for 1 hour. The reaction was concentrated to dryness in vacuo to give 3-(3-((3aS,6aS)-hexahydropyrrolo[3,2-b]pyrrol-1 (2H)-yl)phenyl)piperidine-2,6-dione (40 mg, 74 μmol, 130%, 55.6% purity) as a brown oil. LC-MS (ESI+) m/z: 300.2 (M+H)⁺.

Step 4: A mixture of 1-(4-((1R,2S)-6-hydroxy-2-phenyl-1,2,3,4-tetrahydronaphthalen-1-yl)phenyl)piperidine-4-carbaldehyde (61 mg, 90% Wt, 1 Eq, 0.13 mmol), 3-(3-((3aS,6aS)-hexahydropyrrolo[3,2-b]pyrrol-1 (2H)-yl)phenyl)piperidine-2,6-dione (40 mg, 1 Eq, 0.13 mmol) and sodium acetate (55 mg, 5 Eq, 0.67 mmol) in DCM (2 mL) and MeOH (2 mL) at 20° C. for 0.5 hour, pH=8, then acetic acid (24 mg, 23 μL, 3 Eq, 0.40 mmol) at 20° C. for 1 hour, pH=6, then added Sodium triacetoxyborohydride (57 mg, 40 μL, 2 Eq, 0.27 mmol) the mixture, and then the mixture was stirred at 25° C. for 16 hour. The reaction was quenched with water (50 mL) and extracted with ethyl acetate (50 mL*3). The organic layer was washed with brine (50 mL) and dried over Na₂SO₄ and concentrated in vacuo to give 3-(3-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione as a brown oil. The brown oil was purified by preparative high-performance liquid chromatography. Condition: water(NH₃H₂O+NH₄HCO₃)-CAN, Column: Boston Prime C18 150*30 mm*5 um, at the beginning: A (15%) and B (85%), at the end: A: (0%) and B (100%), Gradient Time (min) 7; 100% B hold Time (min) 2, Flow Rate (ml/min) 25. The pure fractions were collected and the solvent was evaporated under vacuum. The aqueous layer was lyophilized to dryness to give 3-(3-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione (8.2 mg, 12 μmol, 8.8%, 99.48% Purity) as a white solid.

LCMS: calc. for C₄₅H₅₀N₄O₃:694.39, found: [M+H]⁺695.3.

HPLC: 99.48% purity at 220 nm.

¹H NMR (400 MHz, DMSO-d₆) δ=10.80 (d, J=2.6 Hz, 1H), 9.10 (s, 1H), 7.14 (br d, J=7.4 Hz, 4H), 6.83 (br d, J=6.8 Hz, 2H), 6.64 (d, J=8.2 Hz, 1H), 6.59 (d, J=2.1 Hz, 1H), 6.52 (d, J=8.7 Hz, 2H), 6.49 (s, 1H), 6.42 (s, 2H), 6.39-6.34 (m, 1H), 6.19 (d, J=8.6 Hz, 2H), 4.17-4.09 (m, 2H), 3.76-3.69 (m, 1H), 3.50 (br d, J=11.3 Hz, 2H), 3.30-3.20 (m, 3H), 3.10-3.06 (m, 1H), 3.00-2.88 (m, 3H), 2.68-2.56 (m, 2H), 2.47 (br s, 3H), 2.20-2.02 (m, 6H), 1.89-1.81 (m, 2H), 1.80-1.73 (m, 1H), 1.68 (br d, J=2.9 Hz, 2H), 1.57-1.45 (m, 1H), 1.39-1.29 (m, 1H), 1.24-1.10 (m, 2H)

SFC: retention time, 2.265 min, 2.459 min; Area, 45.93%, 54.07%;

### Example 173. Preparation of (I-178) 3-(3-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione

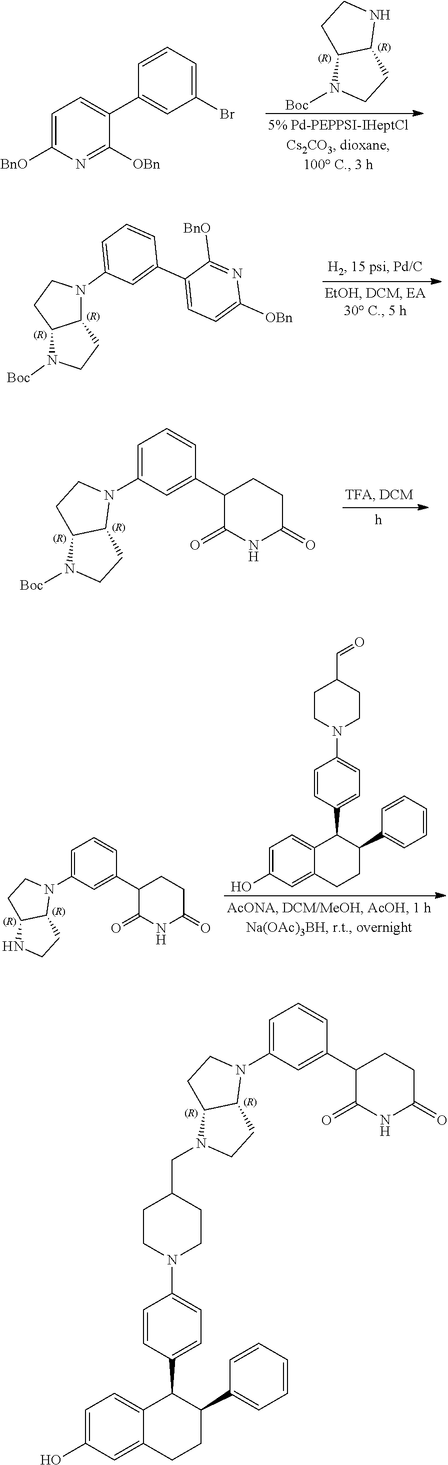

Step 1: A mixture of 2,6-bis(benzyloxy)-3-(3-bromophenyl)pyridine (161.9 mg, 362.7 μmol, 1.1 eq), tert-butyl (3aR,6aR)-2,3,3a,5,6,6a-hexahydro-1H-pyrrolo[3,2-b]pyrrole-4-carboxylate (70 mg, 329.7 μmol, 1 eq), cesium carbonate (322.3 mg, 989.2 μmol, 3 eq), 1,3-bis[2,6-bis(1-propylbutyl)phenyl]-4,5-dichloro-2H-imidazol-1-ium-2-ide;3-chloropyridine;dichloropalladium (32.1 mg, 33 μmol, 0.1 eq) in dioxane (5 mL) was degassed and purged with N₂ for 3 times, and then the mixture was stirred at 100° C. for 3.5 hr under N₂ atmosphere. LCMS showed ˜50% desired MS was detected. Then the mixture was treated with H₂O (20 mL), extracted with EtOAc (50 mL). The combined extracts was dried over anhydrous Na₂SO₄, filtered and concentrated to dryness in vacuo to give a residue. The residue was purified by flash silica gel chromatography (ISCO®; 4 g SepaFlash® Silica Flash Column, Eluent of 0˜20% DCM/MeOH @ 30 mL/min). The pure fractions were collected and concentrated to dryness in vacuo to give tert-butyl (3aR,6aR)-4-(3-(2,6-bis(benzyloxy)pyridin-3-yl)phenyl) hexahydropyrrolo[3,2-b]pyrrole-1 (2H)-carboxylate (90 mg, 155.8 μmol, 47.25% yield, 100% purity) was obtained as a yellow oil. LC-MS (ESI+) m/z: 578.4 (M+H)⁺.

Step 2: A mixture of tert-butyl (3aR,6aR)-1-[3-(2,6-dibenzyloxy-3-pyridyl)phenyl]-2,3,3a,5,6,6a-hexahydropyrrolo[3,2-b]pyrrole-4-carboxylate (90 mg, 155.79 μmol, 1 eq) in DCM (2 mL) ethyl acetate (2 mL) was added Pd/C (10%, 100 mg) under N₂ atmosphere. The suspension was degassed and purged with H₂ for 3 times. The mixture was stirred under H₂ (15 Psi) at 30° C. for 5 hr. LCMS showed 70% desired MS was detected. The reaction mixture was filtered and concentrated to dryness in vacuo to give tert-butyl (3aR,6aR)-1-[3-(2,6-dioxo-3-piperidyl)phenyl]-2,3,3a,5,6,6a-hexahydropyrrolo[3,2-b]pyrrole-4-carboxylate (77 mg, crude) as yellow oil. LC-MS (ESI+) m/z: 400.1 (M+H)+.

Step 3: A mixture of tert-butyl (3aR,6aR)-1-[3-(2,6-dioxo-3-piperidyl)phenyl]-2,3,3a,5,6,6a-hexahydropyrrolo[3,2-b]pyrrole-4-carboxylate (77 mg, 192.75 μmol, 1 eq) in DCM (5 mL) and TFA (1 mL). The mixture was stirred at 30° C. for 1 hr. LCMS showed 64% desired MS was detected. The reaction mixture was filtered and concentrated to dryness in vacuo to give 3-[3-[(3aR,6aR)-2,3,3a,5,6,6a-hexahydro-1H-pyrrolo[3,2-b]pyrrol-4-yl]phenyl]piperidine-2,6-dione (50 mg, 167.02 μmol, 86.65% yield) as yellow oil. LC-MS (ESI+) m/z: 299.9 (M+H)+.

Step 4: A mixture of 3-[3-[(3aR,6aR)-2,3,3a,5,6,6a-hexahydro-1H-pyrrolo[3,2-b]pyrrol-4-yl]phenyl]piperidine-2,6-dione (50 mg, 167 μmol, 1 eq), 1-(4-((1R,2S)-6-hydroxy-2-phenyl-1,2,3,4-tetrahydronaphthalen-1-yl)phenyl)piperidine-4-carbaldehyde (103.1 mg, 250.5 μmol, 1.5 eq) and sodium acetate (82.21 mg, 1.00 mmol, 6 eq) and acetic acid (40.1 mg, 668.1 μmol, 38.3 μL, 4 eq) in DCM (2 mL) and MeOH (2 mL) at 30° C. for 1 hour, then NaB(OAc)₃H (141.6 mg, 668.1 μmol, 4 eq) was added to the mixture and was stirred at 30° C. for 16 hr. LCMS showed 93% desired MS was detected. The reaction mixture was treated with 30 mL H₂O and extracted with EtOAc 50 mL (50 mL*2). The organic layers were dried with anhydrous Na₂SO₄, filtered. The residue was purified by prep. HPLC (column: Welch Xtimate C18 150*30 mm*5 um; mobile phase: [water (FA)-ACN]; gradient: 15%-55% B over 9 min). The aqueous phase was lyophilized to dryness to give 3-(3-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl) piperidine-2,6-dione (40 mg, 39.14 μmol, 23.44% yield, 68% purity) was obtained as a white solid.

LC-MS (ESI+) m/z: 695.5 (M+H)+.

HPLC: 99.109%, purity at 220 nm.

SFC: retention time, 2.292, 2.491 min; Arca, 41.824%, 58.176%;

¹H NMR (400 MHZ, DMSO-d₆) δ=10.85-10.76 (m, 1H), 9.16-9.05 (m, 1H), 7.21-7.05 (m, 4H), 6.88-6.80 (m, 2H), 6.70-6.57 (m, 2H), 6.57-6.35 (m, 6H), 6.20 (d, J=8.6 Hz, 2H), 4.23-4.08 (m, 2H), 3.81-3.66 (m, 1H), 3.59-3.43 (m, 2H), 3.32-3.17 (m, 3H), 3.14-3.06 (m, 1H), 3.03-2.85 (m, 3H), 2.71-2.54 (m, 2H), 2.49-2.40 (m, 3H), 2.26-1.98 (m, 6H), 1.93-1.60 (m, 5H), 1.59-1.44 (m, 1H), 1.35 (br d, J=3.3 Hz, 1H), 1.26-1.09 (m, 2H).

### Example 174. Preparation of (I-177) 3-(4-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione

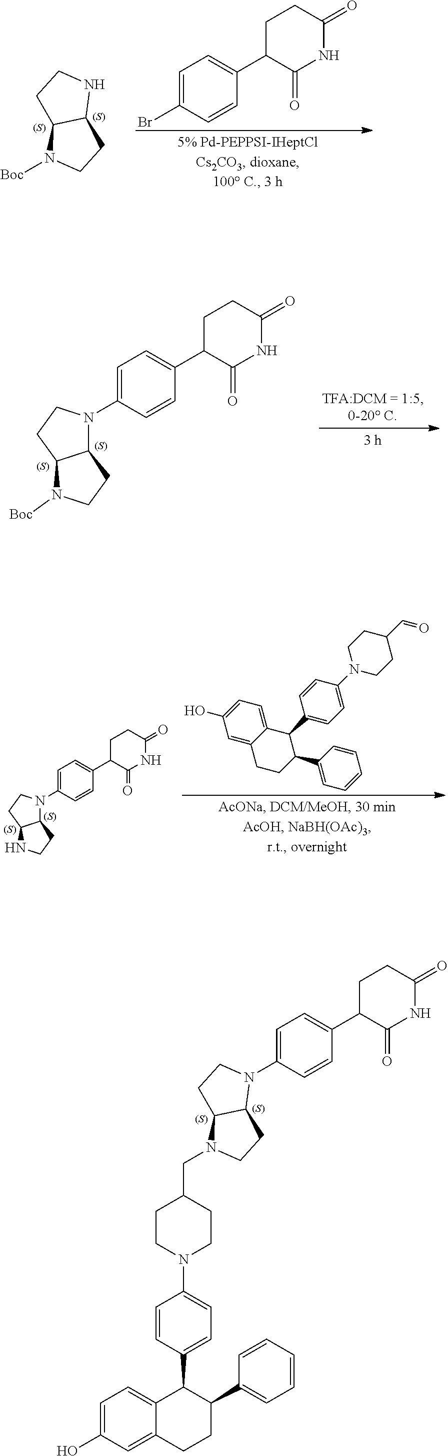

Step 1: A mixture of tert-butyl (3aS,6aS)-hexabydropyrrolo[3,2-b]pyrrole-1 (2H)-carboxylate (100.0 mg, 1 Eq, 471.1 μmol), 3-(4-bromophenyl)piperidine-2,6-dione (138.9 mg, 1.1 Eq, 518.2 μmol), Pd-PEPPSI-IHeptCI (45.82 mg, 0.1 Eq, 47.11 μmol) cesium carbonate (460.4 mg, 3 Eq, 1.413 mmol) in dioxane (2 mL) was degassed and purged with N₂ for 3 times, and then the mixture was stirred at 80° C. for 12 hour under N₂ atmosphere. LCMS showed 25.5% desired MS. TLC (petroleum ether:ethyl acetate=5:1, UV) showed one main new spot was observed. 50 mL water was added, and the mixture was extracted with dichloromethane (10 mL×2). The combined organic layers were washed with brine (10 mL), dried over anhydrous Na₂SO₄, filtered and concentrated to dryness in vacuo to give a yellow oil. The aqueous phase was adjust to pH=˜7 with 1N HCl, and it was discarded. The yellow oil was purified by flash column (petroleum ether/ethyl acetate from 100/0 to 0/100) and the organic layer was concentrated in vacuo to give tert-butyl tert-butyl (3aS,6aS)-4-(4-(2,6-dioxopiperidin-3-yl)phenyl) hexahydropyrrolo[3,2-b]pyrrole-1 (2H)-carboxylate as a yellow solid (100 mg, 250 μmol). LC-MS (ESI+) m/z: 400.3 (M+H)⁺.

Step 2: A solution of tert-butyl (3aS,6aS)-4-(4-(2,6-dioxopiperidin-3-yl)phenyl) hexahydropyrrolo[3,2-b]pyrrole-1 (2H)-carboxylate (100 mg, 1 Eq, 250 μmol) in DCM (2.5 mL) was added TFA (0.7 g, 0.5 mL, 6 mmol) at 0° C. for 5 min. The reaction was stirred at 20° C. for 3 hour. LCMS showed 31.522% desired MS. The reaction was concentrated in vacuo to give 3-(4-((3aS, 6aS)-hexahydropyrrolo[3,2-b]pyrrol-1 (2H)-yl)phenyl)piperidine-2, 6-dione, TFA salt (96 mg, 72 μmol, 29%, 31% purity) as a yellow oil. LC-MS (ESI+) m/z: 300.3 (M+H)⁺.

Step 3: To a solution of 1-(4-((1R,2S)-6-hydroxy-2-phenyl-1,2,3,4-tetrahydronaphthalen-1-yl)phenyl)piperidine-4-carbaldehyde (137 mg, 1 Eq, 334 μmol), 3-(4-((3aS,6aS)-hexahydropyrrolo[3,2-b]pyrrol-1 (2H)-yl)phenyl)piperidine-2,6-dione, TFA salt (96 mg, 72 μmol, 29%, 31% purity) in DCM (2 mL), MeOH (2 mL) was added sodium acetate (82.2 mg, 3 Eq, 1.00 mmol) was stirred at 25° C. for 30 mins, Then sodium triacetoxyborohydride (142 mg, 2 Eq, 668 μmol) and acetic acid (60.2 mg, 57.6 μL, 3 Eq, 1.00 mmol) was added The mixture was stirred at 25° C. for 16 hour. LCMS showed 92% desired MS. The reaction was purified by preparative high-performance liquid chromatography. Condition: Column: Welch Xtimate C18 150*30 mm*5 um; B: ACN; at the beginning: B (14%); at the end: B (54%); Gradient Time (min) 9; 100% B hold Time (min) 2, Flow Rate (ml/min) 30. The pure fractions were collected and the solvent was evaporated under vacuum. The aqueous layer was lyophilized to dryness to give 3-(4-((3aS,6aS)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione (72.7 mg, 105 μmol, 31.3%, 99.9% purity) as a pink solid.

LCMS: calc. for C₄₅H₅₀N₄O₃:694.92, found: [M+H]⁺695.4.

HPLC: 99.855% purity at 220 nm,

¹H NMR (400 MHz, DMSO-d₆) δ=10.76 (s, 1H) 9.12 (s, 1H) 8.14 (s, 0.959H) 7.09-7.20 (m, 3H) 6.99 (br d, J=8.23 Hz, 2H) 6.84 (br d, J=6.56 Hz, 2H) 6.58-6.66 (m, 2H) 6.46-6.56 (m, 5H) 6.20 (br d, J=8.82 Hz, 2H) 4.13 (br d, J=5.01 Hz, 2H) 3.68 (br dd, J=10.43, 4.83 Hz, 1H) 3.51 (br d, J=8.23 Hz, 2H) 3.26 (br s, 6H) 2.89-3.03 (m, 3H) 2.58-2.69 (m, 1H) 2.43-2.49 (m, 3H) 2.03-2.28 (m, 5H) 1.85 (br d, J=12.28 Hz, 3H) 1.69 (br s, 2H) 1.54 (br s, 1H) 1.38 (br s, 1H) 1.09-1.28 (m, 2H)

SFC: retention time, 3.019/7.507 min; Area, 50.911/49.089%;

### Example 175. Preparation of (I-176) 3-(4-((3aR,6aR)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione

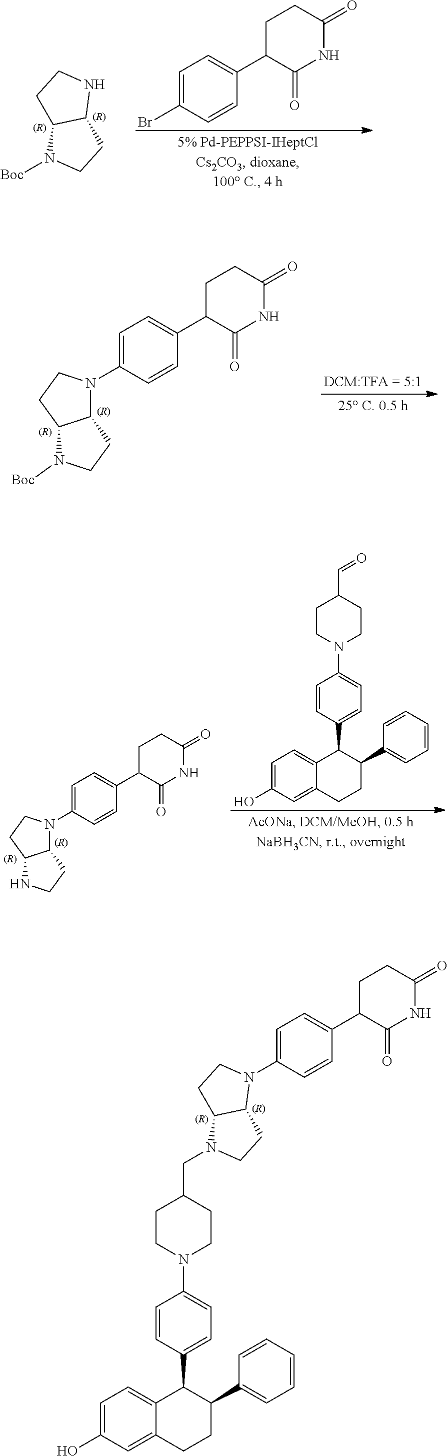

Step 1: A mixture of tert-butyl (3aS,6aS)-2,3,3a,5,6,6a-hexahydro-1H-pyrrolo[3,2-b]pyrrole-4-carboxylate (100 mg, 471.1 μmol, 1 eq), 3-(4-bromophenyl)piperidine-2,6-dione (151.6 mg, 565.3 μmol, 1.2 eq), Pd-PEPPSI-IHeptCI (45.8 mg, 47.1 μmol, 0.1 eq), Cs₂CO₃ (460.4 mg, 1.4 mmol, 3 eq) was added in dioxane (3 mL), the mixture was stirred at 100° C. for 4 hr. LCMS showed ˜38% desired MS was detected. Then the mixture was treated with H₂O (20 mL), extracted with EtOAc (50 mL). The combined extracts was dried over anhydrous Na₂SO₄, filtered and concentrated to dryness in vacuo to give a residue. The residue was subjected to column chromatography over silica gel (gradient elution: 0˜10% ethyl acetate in petroleum ether). The pure fractions were collected and concentrated to dryness in vacuo to give tert-butyl (3aR,6aR)-4-(4-(2,6-dioxopiperidin-3-yl)phenyl) hexahydropyrrolo[3,2-b]pyrrole-1 (2H)-carboxylate (120 mg, 300.39 μmol, 63.77% yield) was obtained as a white solid. LC-MS (ESI+) m/z: 400.01 (M+H)+.

Step 2: A mixture of tert-butyl (3aR,6aR)-4-(4-(2,6-dioxopiperidin-3-yl)phenyl) hexahydropyrrolo[3,2-b]pyrrole-1 (2H)-carboxylate (120 mg, 300.4 μmol, 1 eq) was added in DCM (5 mL) and TFA (1 mL), the mixture was stirred at 25° C. for 0.5 hr, LCMS showed 95% desired MS was detected. The reaction mixture was filtered and concentrated to dryness in vacuo to give 3-(4-((3aR,6aR)-hexahydropyrrolo[3,2-b]pyrrol-1 (2H)-yl)phenyl) piperidine-2,6-dione (85 mg, 283.93 μmol, 94.52% yield). LC-MS (ESI+) m/z: 300 (M+H)+.

Step 3: A mixture of 1-(4-((1R,2S)-6-hydroxy-2-phenyl-1,2,3,4-tetrahydronaphthalen-1-yl)phenyl)piperidine-4-carbaldehyde (123.7 mg, 300.6 μmol, 1 eq), 3-(4-((3aR,6aR)-hexahydropyrrolo[3,2-b]pyrrol-1 (2H)-yl)phenyl)piperidine-2,6-dione (85 mg, 283.9 μmol, 0.95 eq), sodium acetate (147.97 mg, 1.80 mmol, 6 eq) was added in DCM (4 mL) and MeOH (4 mL), the mixture was stirred at 25° C. for 0.5 h, and then sodium; triacetoxyboranuide (254.87 mg, 1.20 mmol, 4 eq), acetic acid (498.1 mg, 1.8 mmol, 474.4 μL, 6 eq) was added in mixture, and was stirred at 25° C. for 12 h. LCMS showed 66% desired MS was detected. The reaction mixture was treated with 30 mL H₂O and extracted with EtOAc (50 mL*2). The organic layers were dried with anhydrous Na₂SO₄, filtered. The residue was purified by prep. HPLC (column: Welch Xtimate C18 150*30 mm*5 um; mobile phase: [water (FA)-ACN]; gradient: 21%-51% B over 7 min). The aqueous phase was lyophilized to dryness to give 3-(4-((3aR,6aR)-4-((1-(4-((1R,2S)-6-hydroxy-2-phenyl-1,2,3,4-tetrahydronaphthalen-1-yl)phenyl)piperidin-4-yl)methyl) hexahydropyrrolo[3,2-b]pyrrol-1 (2H)-yl)phenyl)piperidine-2,6-dione (18.6 mg, 43.17 μmol, 14.36% yield) was obtained as a white solid.

LC-MS (ESI+) m/z: 695.3 (M+H)+.

HPLC: 100%, purity at 220 nm.

SFC: retention time, 3.208, 6.543 min; Area, 50.482%, 49.518%;

¹H NMR (400 MHZ, DMSO-d₆) δ=10.84-10.65 (m, 1H), 9.17-9.03 (m, 1H), 8.15 (s, 0.339H), 7.21-7.08 (m, 3H), 7.02-6.94 (m, 2H), 6.88-6.79 (m, 2H), 6.69-6.58 (m, 2H), 6.57-6.43 (m, 5H), 6.25-6.16 (m, 2H), 4.22-4.09 (m, 2H), 3.73-3.63 (m, 1H), 3.56-3.45 (m, 2H), 3.31-3.18 (m, 4H), 3.16-3.04 (m, 1H), 3.04-2.83 (m, 3H), 2.71-2.56 (m, 1H), 2.49-2.39 (m, 3H), 2.33-1.95 (m, 6H), 1.94-1.61 (m, 5H), 1.60-1.29 (m, 2H), 1.28-1.06 (m, 2H).

### Example 176. Preparation of (I-266) (S)-3-(5-(4-((1-(4-((1S,1′R)-6′-hydroxy-3,3′,4,4′-tetrahydro-1′H,2H-1,2′-spirobi[naphthalen]-1′-yl)phenyl)piperidin-4-yl)methyl)piperazin-1-yl)-1-oxoisoindolin-2-yl)piperidine-2,6-dione

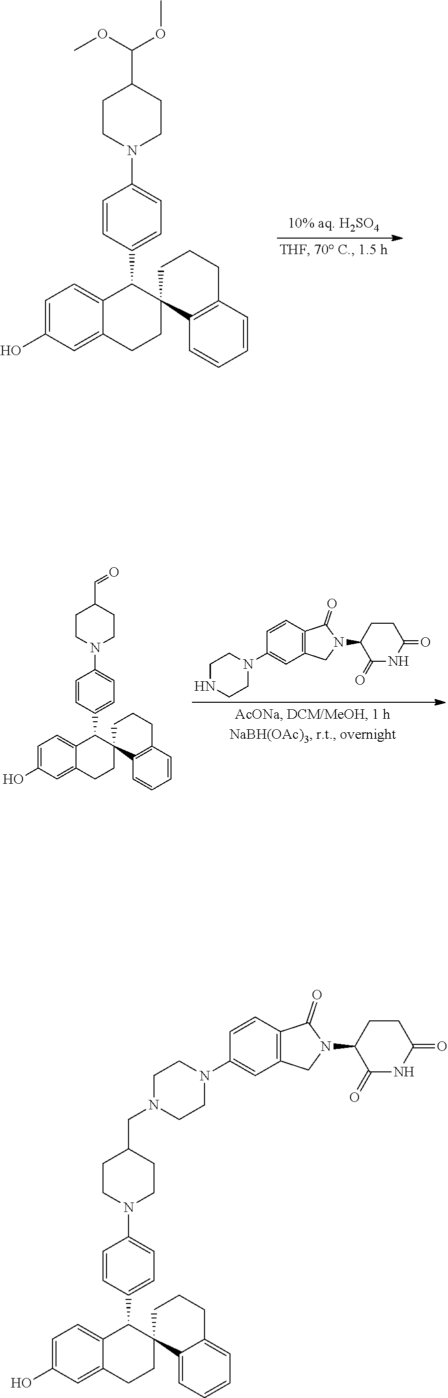 [SMILES: COC(OC)C1CCN(c2ccc([C@H]3c4ccc(O)cc4CC[C@@]34CCCc3ccccc34)cc2)CC1 ; O=CC1CCN(c2ccc([C@H]3c4ccc(O)cc4CC[C@@]34CCCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@@]78CCCc7ccccc78)cc6)CC5)CC4)ccc3C2=O)C(=O)N1]

Step 1: To a solution of (1S,1′R)-1′-(4-(4-(dimethoxymethyl)piperidin-1-yl)phenyl)-3,3′,4,4′-tetrahydro-1′H,2H-1,2′-spirobi[naphthalen]-6′-ol (25.0 mg, 1 Eq, 50.2 μmol) in THF (1.5 mL) and 10% H₂SO₄ (1.5 mL). The mixture was stirred at 70° C. for 1.5 hour. LCMS showed 95% desired MS was detected. The reaction mixture was quenched by addition H₂O 50 mL at 25° C., and then diluted with 50 mL H₂O and extracted with Ethyl acetate 30 mL (50 mL*2). The combined organic layers were washed with brine 10 mL. dried over Na₂SO₄, filtered and concentrated under reduced pressure to give 1-(4-((1S, 1′R)-6′-hydroxy-3,3′,4,4′-tetrahydro-1′H,2H-1,2′-spirobi[naphthalen]-1′-yl)phenyl)piperidine-4-carbaldehyde (25.0 mg, 53 μmol, 100%, 95% purity) as a white oil. LC-MS (ESI+) m/z: 452.2 (M+H)⁺.

Step 2: To a solution of 1-(4-((1S, 1′R)-6′-hydroxy-3,3′,4,4′-tetrahydro-1′H,2H-1,2′-spirobi[naphthalen]-1′-yl)phenyl)piperidine-4-carbaldehyde (25 mg, 1 Eq, 55.4 μmol), (S)-3-(1-oxo-5-(piperazin-1-yl)isoindolin-2-yl)piperidine-2,6-dione, Benzene sulfonic acid (32.3 mg, 1.2 Eq. 66.4 μmol) in DCM (1 mL), MeOH (1 mL) was added sodium acetate (13.6 mg, 3 Eq, 166.1 μmol) was stirred at 25° C. for 60 min, Then sodium triacetoxyhydroborate (23.5 mg, 2 Eq, 110.7 μmol) and acetic acid (10 mg, 3 Eq, 166.1 μmol) was added. The mixture was stirred at 25° C. for 16 hour. LCMS showed 96% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 15%-55%,9 min to give (S)-3-(5-(4-((1-(4-((1S, 1′R)-6′-hydroxy-3,3′,4,4′-tetrahydro-1′H,2H-1,2′-spirobi[naphthalen]-1′-yl)phenyl)piperidin-4-yl)methyl)piperazin-1-yl)-1-oxoisoindolin-2-yl)piperidine-2,6-dione (10.4 mg, 13.4 μmol, 24.2%, 98.4% purity) as a white solid.

LCMS: calc. for C₄₈H₅₃N₅O₄:763.98, found: [M+H]⁺764.4.

HPLC: 98.398% purity at 220 nm.

¹H NMR 1H NMR (400 MHz, DMSO-d₆) δ=10.96 (s, 1H) 9.12 (br s, 0.492H) 8.21 (s, 1H) 7.54 (dd, J=17.32, 8.03 Hz, 2H) 7.10-7.16 (m, 1H) 7.01-7.08 (m, 3H) 6.93 (d, J=7.28 Hz, 1H) 6.68 (s, 4H) 6.54-6.60 (m, 2H) 6.43-6.49 (m, 1H) 5.05 (dd, J=13.30, 5.02 Hz, 1H) 4.52 (s, 1H) 4.28-4.37 (m, 1H) 4.16-4.24 (m, 1H) 3.50-3.63 (m, 2H) 3.28 (br s, 8H) 2.91 (brt, J=13.05 Hz, 2H) 2.54-2.62 (m, 4H) 2.29-2.45 (m, 3H) 2.21 (br d, J=7.28 Hz, 2H) 1.95-2.09 (m, 2H) 1.65-1.82 (m, 5H) 1.43-1.57 (m, 2H) 1.17 (br d, J=10.04 Hz, 3H).

SFC: retention time, 3.011 min; Area, 96.476%;

### Example 177. Preparation of (I-265) (S)-3-(5-(4-((1-(4-((1R,1′S)-6′-hydroxy-3,3′,4,4′-tetrahydro-1′H,2H-1,2′-spirobi[naphthalen]-1′-yl)phenyl)piperidin-4-yl)methyl)piperazin-1-yl)-1-oxoisoindolin-2-yl)piperidine-2,6-dione

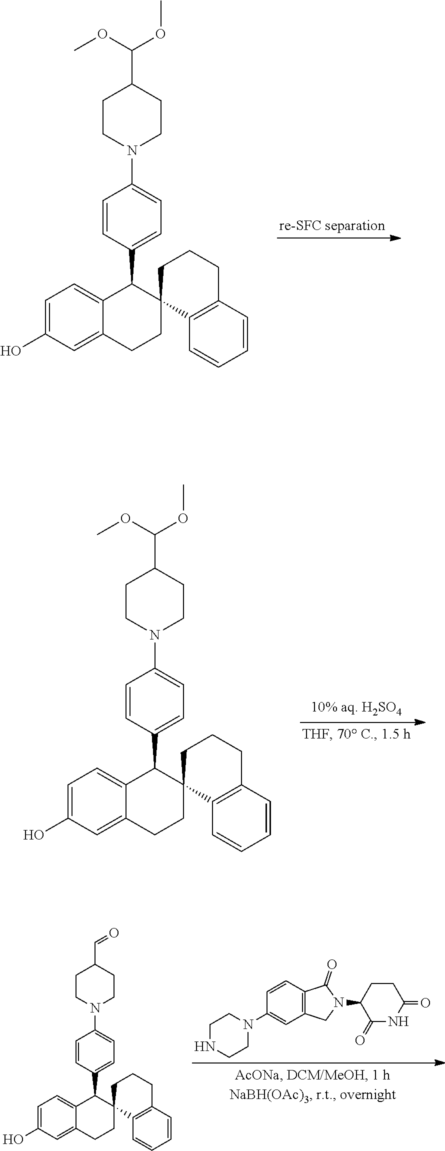 [SMILES: COC(OC)C1CCN(c2ccc([C@@H]3c4ccc(O)cc4CC[C@]34CCCc3ccccc34)cc2)CC1 ; COC(OC)C1CCN(c2ccc([C@@H]3c4ccc(O)cc4CC[C@]34CCCc3ccccc34)cc2)CC1 ; O=CC1CCN(c2ccc([C@@H]3c4ccc(O)cc4CC[C@]34CCCc3ccccc34)cc2)CC1 ; O=C1CC[C@H](N2Cc3cc(N4CCNCC4)ccc3C2=O)C(=O)N1]

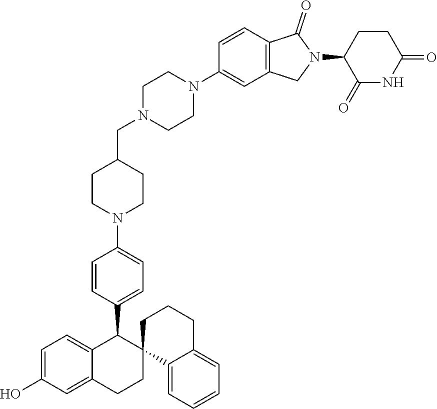 [SMILES: O=C1CC[C@H](N2Cc3cc(N4CCN(CC5CCN(c6ccc([C@@H]7c8ccc(O)cc8CC[C@]78CCCc7ccccc78)cc6)CC5)CC4)ccc3C2=O)C(=O)N1]

Step 1: The starting material was concentrated under reduced pressure to give desired compound as a yellow oil, which was further separated by SFC (condition: column: DAICEL CHIRALPAK AD (250 mm*30 mm, 10 um)); mobile phase: CO₂-EtOH (0.1% NH₃H₂O); B %: 30%-30%) to give (1R, 1′S)-1′-(4-(4-(dimethoxymethyl)piperidin-1-yl)phenyl)-3,3′,4,4′-tetrahydro-1′H,2H-1,2′-spirobi[naphthalen]-6′-ol (28.0 mg, 56.3 μmol, 93.3%) as a white solid.

Step 2: To a solution of (1R, 1′S)-1′-(4-(4-(dimethoxymethyl)piperidin-1-yl)phenyl)-3,3′,4,4′-tetrahydro-1′H,2H-1,2′-spirobi[naphthalen]-6′-ol (28.00 mg, 1 Eq, 56.26 μmol) in THF (1.5 mL) and 10% H₂SO₄ (1.5 mL). The mixture was stirred at 70° C. for 1.5 hour. LCMS showed 94% desired MS was detected. The reaction mixture was quenched by addition H₂O 50 mL at 25° C., and then diluted with 50 mL H₂O and extracted with Ethyl acetate 30 mL (50 mL*2). The combined organic layers were washed with brine 10 mL. dried over Na₂SO₄, filtered and concentrated under reduced pressure to give 1-(4-((1R, 1′S)-6′-hydroxy-3,3′,4,4′-tetrahydro-1′H,2H-1,2′-spirobi[naphthalen]-1′-yl)phenyl)piperidine-4-carbaldehyde (22.0 mg, 46 μmol, 81%, 94% purity) as a residue. LC-MS (ESI+) m/z: 452.2 (M+H)⁺.

Step 3: To a solution of 1-(4-((1R, 1′S)-6′-hydroxy-3,3′,4,4′-tetrahydro-1′H,2H-1,2′-spirobi[naphthalen]-1′-yl)phenyl)piperidine-4-carbaldehyde (22. mg, 1 Eq, 48.7 μmol), (S)-3-(1-oxo-5-(piperazin-1-yl)isoindolin-2-yl)piperidine-2,6-dione Benzene sulfonic acid (19.2 mg, 1.2 Eq. 58.5 μmol) in DCM (2 mL), MeOH (2 mL) was added sodium acetate (12 mg, 3 Eq, 146.1 μmol) was stirred at 25° C. for 60 min, Then sodium triacetoxyhydroborate (20.7 mg, 2 Eq, 97.4 μmol) and acetic acid (8.8 mg, 8.4 μL, 3 Eq, 146.1 μmol) was added. The mixture was stirred at 25° C. for 16 hour. LCMS showed 85% desired MS was detected. The reaction mixture was concentrated under reduced pressure to remove solvent. The residue was purified by prep-HPLC (column: Welch Xtimate C18 150*30 mm+5 um phase: [water (FA)-ACN]; B %: 10%-50%,9 min to give (S)-3-(5-(4-((1-(4-((1R,1′S)-6′-hydroxy-3,3′,4,4′-tetrahydro-1′H,2H-1,2′-spirobi[naphthalen]-1′-yl)phenyl)piperidin-4-yl)methyl)piperazin-1-yl)-1-oxoisoindolin-2-yl)piperidine-2,6-dione (12.9 mg, 16.6 μmol, 34.2%, 98.6% purity) as a white solid.

LCMS: calc. for C48H₅₃N₅O₄: 763.98, found: [M+H]⁺764.5.

HPLC: 98.641% purity at 220 nm.

¹H NMR ¹H NMR (400 MHZ, DMSO-d₆) δ=10.96 (s, 1H) 9.11 (br s, 1H) 8.19 (s, 0.539H) 7.54 (dd, J=17.07, 8.28 Hz, 2H) 7.13 (t, J=7.40 Hz, 1H) 7.00-7.08 (m, 3H) 6.93 (d, J=7.03 Hz, 1H) 6.65-6.73 (m, 4H) 6.53-6.59 (m, 2H) 6.46 (dd, J=8.53, 2.51 Hz, 1H) 5.05 (dd, J=13.43, 4.89 Hz, 1H) 4.52 (s, 1H) 4.28-4.37 (m, 1H) 4.15-4.24 (m, 1H) 3.58 (br d, J=10.29 Hz, 2H) 3.28 (br s, 8H) 2.86-2.97 (m, 2H) 2.54-2.62 (m, 4H) 2.29-2.47 (m, 3H) 2.21 (br d, J=7.03 Hz, 2H) 1.94-2.08 (m, 2H) 1.59-1.82 (m, 5H) 1.41-1.58 (m, 2H) 1.17 (br d, J=10.54 Hz, 3H).

SFC: retention time, 2.985 min; Area, 94.619%;

### Example 178. Preparation of (I-264) (S)-3-(5-(4-((1-(4-((1S,1′S)-6′-hydroxy-3,3′,4,4′-tetrahydro-1′H,2H-1,2′-spirobi[naphthalen]′-yl)phenyl)piperidin-4-yl)methyl)piperazin-1-yl)-1-oxoisoindolin-2-yl)piperidine-2,6-dione

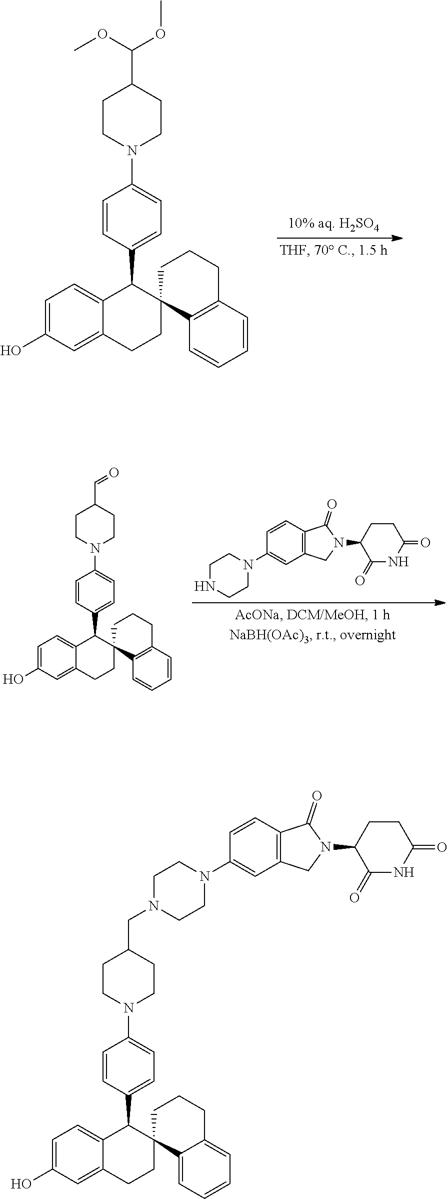 [SMILES: COC(OC)C1CCN(c2ccc([C@@H]3c4ccc(O)cc4CC[C@@]34CCCc3ccccc34)cc2)CC1 ; O=CC1CCN(c2ccc([C@@H]3c4ccc(O)cc4CC[C@@]34CCCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CC[C@@]78CCCc7ccccc78)cc6)CC5)CC4)ccc3C2=O)C(=O)N1]

Step 1: To a solution of (1S,1′S)-1′-(4-(4-(dimethoxymethyl)piperidin-1-yl)phenyl)-3,3′,4,4′-tetrahydro-1′H,2H-1,2′-spirobi[naphthalen]-6′-ol (100. mg, 1 Eq, 200.9 μmol) in THF (5 mL) and 10% H₂SO₄ (5 mL). The mixture was stirred at 70° C. for 1.5 hour. LCMS showed 100% desired MS was detected. The reaction mixture was quenched by addition H₂O 50 mL at 25° C., and then diluted with 50 mL H₂O and extracted with Ethyl acetate 30 mL (50 mL*2). The combined organic layers were washed with brine 10 mL. dried over Na₂SO₄, filtered and concentrated under reduced pressure to give 1-(4-((1S, 1′S)-6′-hydroxy-3,3′,4,4′-tetrahydro-1′H,2H-1,2′-spirobi[naphthalen]-1′-yl)phenyl)piperidine-4-carbaldehyde (98.0 mg, 217 μmol, 108%, 100% purity) as a residue. LC-MS (ESI+) m/z: 470.1 (M+H)⁺.

Step 2: To a solution of 1-(4-((1S, 1′S)-6′-hydroxy-3,3′,4,4′-tetrahydro-1′H,2H-1,2′-spirobi[naphthalen]-1′-yl)phenyl)piperidine-4-carbaldehyde (100 mg, 1 Eq, 221.43 μmol), (S)-3-(1-oxo-5-(piperazin-1-yl)isoindolin-2-yl)piperidine-2,6-dione, Benzene sulfonic acid (87.3 mg, 1.2 Eq, 265.7 μmol) in DCM (2 mL), MeOH (2 mL) was added sodium acetate (54.5 mg, 3 Eq, 664.3 μmol) was stirred at 25° C. for 60 min, Then sodium triacetoxyhydroborate (93.9 mg, 2 Eq, 442.9 μmol) and acetic acid (39.9 mg, 3 Eq, 664.3 μmol) was added. The mixture was stirred at 25° C. for 16 hour. LCMS showed 95% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 18%-58%,9 min to give (S)-3-(5-(4-((1-(4-((1S,1′S)-6′-hydroxy-3,3′,4,4′-tetrahydro-1′H,2H-1,2′-spirobi[naphthalen]′-yl)phenyl)piperidin-4-yl)methyl)piperazin-1-yl)-1-oxoisoindolin-2-yl)piperidine-2,6-dione (64.3 mg, 83.7 μmol, 37.8%, 99.5% purity) was obtained as a white solid.

LCMS: calc. for C₄₈H₅₃N₅O₄:763.98, found: [M+H]⁺764.4.

HPLC: 99.457% purity at 220 nm.

¹H NMR 1H NMR (400 MHZ, DMSO-d₆) δ=10.96 (s, 1H) 9.09 (br s, 1H) 8.14 (s, 0.498H) 7.53 (d, J=8.58 Hz, 1H) 7.00-7.10 (m, 3H) 6.91-6.98 (m, 1H) 6.54-6.63 (m, 5H) 6.46 (dd, J=8.23, 2.38 Hz, 1H) 6.27 (br d, J=8.23 Hz, 2H) 6.04 (d, J=7.87 Hz, 1H) 5.06 (dd, J=13.29, 5.07 Hz, 1H) 4.28-4.37 (m, 1H) 4.16-4.25 (m, 1H) 4.01 (s, 1H) 3.54 (br d, J=11.44 Hz, 2H) 3.30 (br s, 6H) 2.85-2.97 (m, 4H) 2.73-2.82 (m, 1H) 2.61 (br s, 6H) 2.34-2.43 (m, 1H) 2.23 (br s, 2H) 1.90-2.02 (m, 3H) 1.76 (br d, J=10.37 Hz, 3H) 1.67 (br s, 1H) 1.43-1.53 (m, 1H) 1.33 (br d, J=8.70 Hz, 1H) 1.14-1.25 (m, 2H)

SFC: retention time, 2.640 min; Area, 96.850%.

### Example 179. Preparation of (I-263) (S)-3-(5-(4-((1-(4-((1R,1′R)-6′-hydroxy-3,3′,4,4′-tetrahydro-1′H,2H-1,2′-spirobi[naphthalen]-1′-yl)phenyl)piperidin-4-yl)methyl)piperazin-1-yl)-1-oxoisoindolin-2-yl)piperidine-2,6-dione

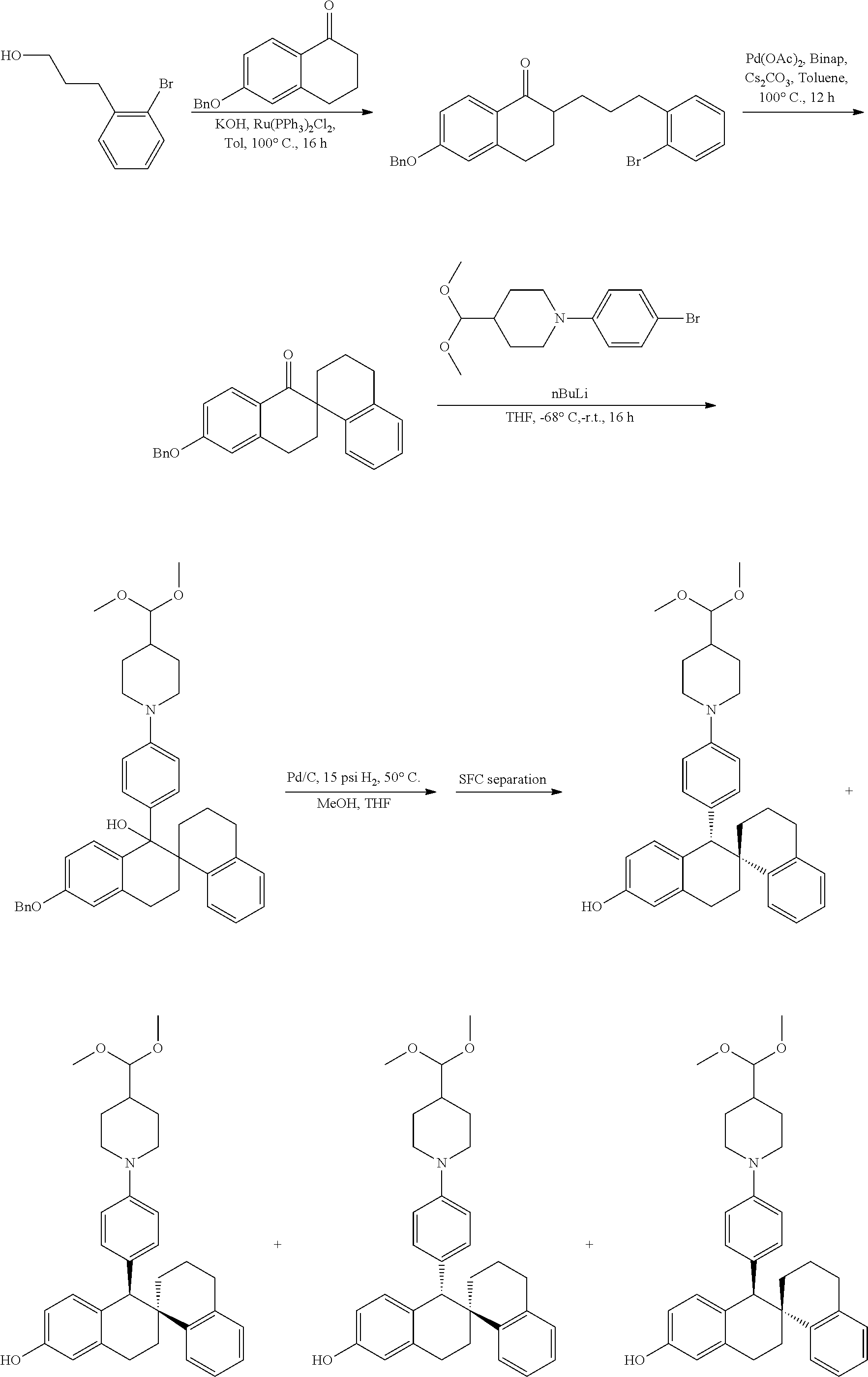

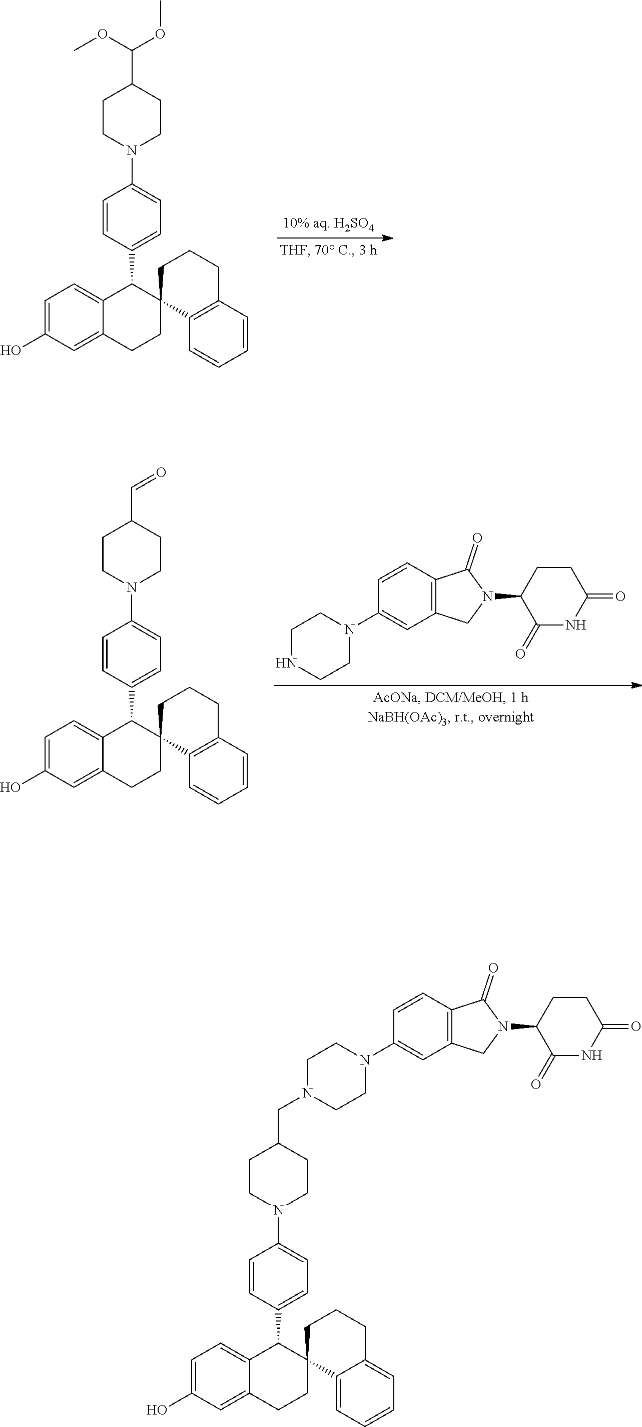 [SMILES: COC(OC)C1CCN(c2ccc([C@H]3c4ccc(O)cc4CC[C@]34CCCc3ccccc34)cc2)CC1 ; O=CC1CCN(c2ccc([C@H]3c4ccc(O)cc4CC[C@]34CCCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@]78CCCc7ccccc78)cc6)CC5)CC4)ccc3C2=O)C(=O)N1]

Step 1: A 100 mL thread vial equipped with magnetic stirrer.

Tris(triphenylphosphine)ruthenium(II)Dichloride (760 mg, 0.1 Eq, 793 μmol), potassium hydroxide (445 mg, 1 Eq, 7.93 mmol),3-(2-bromophenyl) propan-1-ol (2.05 g, 1.2 Eq, 9.51 mmol) and 6-(benzyloxy)-3,4-dihydronaphthalen-1 (2H)-one (2.00 g, 1 Eq, 7.93 mmol) was added in toluene (20 mL) at 100° C. for 16 hour under N₂ atmosphere. TLC (petroleum ether:ethyl acetate=10:1, UV) showed one main new spot was observed. 50 mL water was added, and the mixture was extracted with dichloromethane (100 mL×2). The combined organic layers were washed with brine (50 mL), dried over anhydrous Na₂SO₄, filtered and concentrated to dryness in vacuum to give a yellow oil. The yellow oil was purified by flash column (petroleum ether/ethyl acetate from 100/0 to 0/100) and the organic layer was concentrated in vacuo to give 6-(benzyloxy)-2-(3-(2-bromophenyl) propyl)-3, 4-dihydronaphthalen-1 (2H)-one (1.9 g, 3.4 mmol, 42%, 79.4% purity) as a yellow solid. LC-MS (ESI+) m/z: 451.2 (M+H)⁺.

Step 2: To a mixture of 6-(benzyloxy)-2-(3-(2-bromophenyl) propyl)-3,4-dihydronaphthalen-1 (2H)-one (1 g, 1 Eq, 2.23 mmol), Pd(OAc)₂ (50. mg, 0.1 Eq, 223 μmol) and 2,2′-bis(diphenylphosphaneyl)-1, l′-binaphthalene (139 mg, 0.1 Eq, 223 μmol) and cesium carbonate (2.18 g, 3 Eq, 6.68 mmol) in 1,4-Dioxane (10 mL) was stirred at 100° C. for 12 hour under N₂. TLC (petroleum ether:ethyl acetate=5:1, UV) showed one main new spot was observed. 100 mL water was added, and the mixture was extracted with dichloromethane (200 mL×2). The combined organic layers were washed with brine (100 mL), dried over anhydrous Na₂SO₄, filtered and concentrated to dryness in vacuo to give a yellow oil. The yellow oil was purified by preparative high-performance liquid chromatography. Condition: Column: Xtimate C18 150*40 mm*5 um A: water (FA) B: ACN; at the beginning: A (45%) and B (55%); at the end: A: (5%) and B (95%); Gradient Time (min) 9; 100% B hold Time (min) 2, Flow Rate (ml/min) 30. The pure fractions were collected and the solvent was evaporated under vacuum. The aqueous layer was lyophilized to dryness to give 6′-(benzyloxy)-3,3′,4,4′-tetrahydro-1′H,2H-1,2′-spirobi[naphthalen]-1′-one (450 mg, 1.22 mmol, 54.9%) as a yellow solid.

Step 3: To a solution of 1-(4-bromophenyl)-4-(dimethoxymethyl) piper dine (1.1 g, 3 Eq, 3.5 mmol) in THF (20 mL) was added n-Butyl lithium, 2.5 M in hexane (186.9 mg, 1.167 mL, 2.5 molar, 2.5 Eq, 2.9174 mmol) at −68° C. over 5 min. After addition, the mixture was stirred at −68° C. for 1 hour, and then 6′-(benzyloxy)-3,3′,4,4′-tetrahydro-1′H,2H-1,2′-spirobi[naphthalen]-1′-one (430 mg, 1 Eq, 1.1670 mmol) was added dropwise at −68° C. The resulting mixture was stirred at 25° C. for 16 hour. TLC (petroleum ether:ethyl acetate=5:1, UV) showed one main new spot was observed. The reaction was quenched with water (200 mL) and extracted with ethyl acetate (200 mL*2). The organic layer was washed with brine (100 mL) and dried over Na₂SO₄ and concentrated in vacuo to give a yellow oil. The white solid was subjected to column chromatography over silica gel (20 g gradient elution: 0-30% EtOAc). The desired fractions were collected, and concentrated to dryness in vacuo to give 6′-(benzyloxy)-1′-(4-(4-(dimethoxymethyl)piperidin-1-yl)phenyl)-3,3′,4,4′-tetrahydro-1′H,2H-1,2′-spirobi[naphthalen]-1′-ol (500 mg, 828 μmol, 71.0%) as a yellow solid. LC-MS (ESI+) m/z: 606.4 (M+H)⁺.

Step 4: To a solution of 6′-(benzyloxy)-1′-(4-(4-(dimethoxymethyl)piperidin-1-yl)phenyl)-3,3′,4,4′-tetrahydro-1′H,2H-1,2′-spirobi[naphthalen]-1′-ol (500 mg, 1 Eq, 828.09 μmol) in MeOH (5 mL) and THF (5 mL) was added palladium on carbon (500 mg, 10% Wt., 0.567 Eq, 470 μmol) under N₂ atmosphere. The suspension was degassed and purged with dihydrogen for 3 mines. The mixture was stirred under 15 Psi dihydrogen (1.67 mg, 1 Eq, 828.09 μmol) at 50° C. for 16 hr. LCMS showed 74.512% desired MS was detected. The mixture was filtered and concentrated under reduced pressure to give a residue. The residue was concentrated under reduced pressure to give desired compound as a yellow oil, which was further separated by SFC (condition: column: DAICEL CHIRALPAK AD (250 mm*30 mm, 10 um)); mobile phase: CO₂-EtOH (0.1% NH₃H₂O); B %: 35%-35%) to give (1R, 1′R)-1′-(4-(4-(dimethoxymethyl)piperidin-1-yl)phenyl)-3,3′,4,4′-tetrahydro-1′H,2H-1,2′-spirobi[naphthalen]-6′-ol (160.0 mg, 321.5 μmol, 38.82%) and (1S, 1′S)-1′-(4-(4-(dimethoxymethyl)piperidin-1-yl)phenyl)-3,3′,4,4′-tetrahydro-1′H,2H-1,2′-spirobi[naphthalen]-6′-ol (150.0 mg, 301.4 μmol, 36.40%) and (1S,1′R)-1′-(4-(4-(dimethoxymethyl)piperidin-1-yl)phenyl)-3,3′,4,4′-tetrahydro-1′H,2H-1,2′-spirobi[naphthalen]-6′-ol (35.0 mg, 70.3 μmol, 8.49%) and (1R,1′S)-1′-(4-(4-(dimethoxymethyl)piperidin-1-yl)phenyl)-3,3′,4,4′-tetrahydro-1′H,2H-1,2′-spirobi[naphthalen]-6′-ol (25.0 mg, 50.2 μmol, 6.07%) as a white solid.

Step 5: To a solution of (1R, 1′R)-1′-(4-(4-(dimethoxymethyl)piperidin-1-yl)phenyl)-3,3′,4,4′-tetrahydro-1′H,2H-1,2′-spirobi[naphthalen]-6′-ol (100.0 mg, 1 Eq, 200.9 μmol) in THF (5 mL) and 10% H₂SO₄ (5 mL). The mixture was stirred at 70° C. for 1.5 hour. LCMS showed 100% desired MS was detected. The reaction mixture was quenched by addition H₂O 50 mL at 25° C., and then diluted with 50 mL H₂O and extracted with Ethyl acetate 30 mL (50 mL*2). The combined organic layers were washed with brine 10 mL. dried over Na₂SO₄, filtered and concentrated under reduced pressure to give 1-(4-((1R,1′R)-6′-hydroxy-3,3′,4,4′-tetrahydro-1′H,2H-1,2′-spirobi[naphthalen]-1′-yl)phenyl)piperidine-4-carbaldehyde (102 mg, 226 μmol, 112%, 100% purity) as a residue. LC-MS (ESI+) m/z: 470.2 (M+H)⁺.

Step 6: To a solution of 1-(4-((1R, 1′R)-6′-hydroxy-3,3′,4,4′-tetrahydro-1′H,2H-1,2′-spirobi[naphthalen]-1′-yl)phenyl)piperidine-4-carbaldehyde (100 mg, 1 Eq, 221.4 μmol), (S)-3-(1-oxo-5-(piperazin-1-yl)isoindolin-2-yl)piperidine-2,6-dione, Benzene sulfonic acid (129.3 mg, 1.2 Eq, 265.7 μmol) in DCM (2 mL), MeOH (2 mL) was added sodium acetate (54.5 mg, 3 Eq, 664.3 μmol) was stirred at 25° C. for 60 min, Then sodium triacetoxyborohydride (93.9 mg, 2 Eq, 442.86 μmol) and acetic acid (39.9 mg, 38.2 μL, 3 Eq, 664.29 μmol) was added The mixture was stirred at 25° C. for 16 hour. LCMS showed 92% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 12%-52%,9 min to give (S)-3-(5-(4-((1-(4-((1R, 1′R)-6′-hydroxy-3,3′,4,4′-tetrahydro-1′H,2H-1,2′-spirobi[naphthalen]-1′-yl)phenyl)piperidin-4-yl)methyl)piperazin-1-yl)-1-oxoisoindolin-2-yl)piperidine-2,6-dione (142.6 mg, 185 μmol, 83.5%, 99.1% purity) was obtained as a white solid.

LCMS: calc. for C₄₈H₅₃N₅O₄:763.98, found: [M+H]⁺764.4.

HPLC: 99.123% purity at 220 nm.

¹H NMR 1H NMR (400 MHz, DMSO-d₆) δ=10.96 (s, 1H) 9.09 (br s, 1H) 8.14 (s, 0.648H) 7.52 (d, J=8.46 Hz, 1H) 7.00-7.10 (m, 3H) 6.91-6.98 (m, 1H) 6.54-6.65 (m, 5H) 6.46 (dd, J=8.23, 2.38 Hz, 1H) 6.27 (br d, J=7.99 Hz, 2H) 6.04 (d, J=7.99 Hz, 1H) 5.05 (dd, J=13.23, 5.13 Hz, 1H) 4.33 (br d, J=16.81 Hz, 1H) 4.16-4.27 (m, 1H) 4.01 (s, 1H) 3.54 (br d, J=10.85 Hz, 2H) 3.29 (br s, 8H) 2.91 (br s, 3H) 2.84-2.96 (m, 1H) 2.78 (br dd, J=16.81, 6.56 Hz, 1H) 2.54-2.59 (m, 4H) 2.34-2.42 (m, 1H) 2.23 (br d, J=5.96 Hz, 2H) 1.96 (br d, J=10.37 Hz, 3H) 1.76 (br d, J=10.37 Hz, 3H) 1.66 (br s, 1H) 1.42-1.53 (m, 1H) 1.33 (br d, J=8.94 Hz, 1H) 1.14-1.25 (m, 2H)

SFC: retention time, 2.511 min; Area, 96.141%;

### Example 180. Preparation of (I-262) (S)-3-(5-(4-((1-(4-((1R,1′S)-6′-hydroxy-2,3,3′,4′-tetrahydro-1′H-spiro[indene-1,2′-naphthalen]-1′-yl)phenyl)piperidin-4-yl)methyl)piperazin-1-yl)-1-oxoisoindolin-2-yl)piperidine-2,6-dione

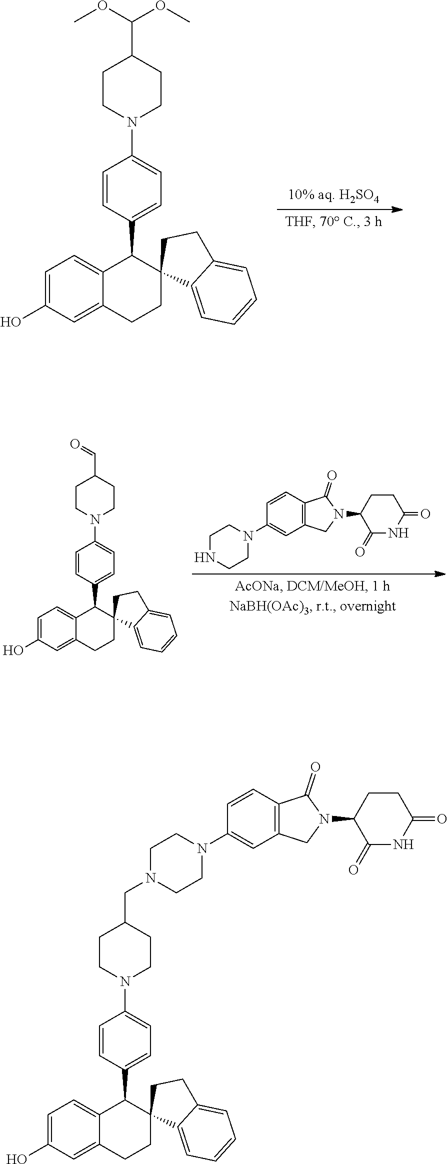 [SMILES: COC(OC)C1CCN(c2ccc([C@@H]3c4ccc(O)cc4CC[C@]34CCc3ccccc34)cc2)CC1 ; O=CC1CCN(c2ccc([C@@H]3c4ccc(O)cc4CC[C@]34CCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CC[C@]78CCc7ccccc78)cc6)CC5)CC4)ccc3C2=O)C(=O)N1]

Step 1:1-(4-((1R, 1′S)-6′-hydroxy-2,3,3′,4′-tetrahydro-1′H-spiro[indene-1,2′-naphthalen]-1′-yl)phenyl)piperidine-4-carbaldehyde (90 mg, 0.20 mmol, 100%, 95.363% purity) in sulfuric acid (187 mg, 3 mL, 10% Wt, 1 Eq, 190 μmol) and THE (3 mL) for stirred at 70° C. for 16 hour. LCMS showed 95.363% desired MS. The reaction was adjust to pH=˜7 with NaHCO₃, then 50 mL water was added, and the mixture was extracted with dichloromethane (100 mL×2). The combined organic layers were washed with brine (100 mL), dried over anhydrous Na₂SO₄, filtered and concentrated to dryness in vacuum to give a yellow oil. The yellow oil was concentrated in vacuum to give 1-(4-((1R, 1′S)-6′-hydroxy-2, 3, 3′, 4′-tetrahydro-1′H-spiro[indene-1, 2′-naphthalen]-1′-yl)phenyl)piperidine-4-carbaldehyde (90 mg, 0.20 mmol, 100%, 95.363% purity) as a yellow solid. LC-MS (ESI+) m/z: 438.1 (M+H)⁺.

Step 2: To a solution of 1-(4-((1R, 1′S)-6′-hydroxy-2,3,3′,4′-tetrahydro-1′H-spirofindene-1,2′-naphthalen]-1′-yl)phenyl)piperidine-4-carbaldehyde (90.0 mg, 1 Eq, 206 μmol), (S)-3-(1-oxo-5-(piperazin-1-yl)isoindolin-2-yl)piperidine-2,6-dione, Benzenesulfonic acid salt (123 mg, 1.2 Eq, 247 μmol) in DCM (2 mL), MeOH (2 mL) was added sodium acetate (50.6 mg, 3 Eq, 617 μmol) was stirred at 25° C. for 60 mines, Then sodium triacetoxyborohydride (87.2 mg, 2 Eq, 411 μmol) and acetic acid (37.1 mg, 35.5 μL, 3 Eq, 617 μmol) was added The mixture was stirred at 25° C. for 16 hour. LCMS showed 87.956% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 10%-50%,9 min to give (S)-3-(5-(4-((1-(4-((1R, 1′S)-6′-hydroxy-2,3,3′,4′-tetrahydro-1′H-spiro[indene-1,2′-naphthalen]-1′-yl)phenyl)piperidin-4-yl)methyl)piperazin-1-yl)-1-oxoisoindolin-2-yl)piperidine-2,6-dione (51.2 mg, 68.3 μmol, 33.2%, 100% purity) was obtained as a white solid. LC-MS (ESI+) m/z: 750.4 (M+H)⁺.

LCMS: calc. for C₄₇H₅₁N₅O₄, found: [M+H]⁺750.2.

HPLC: 100% purity at 220 nm.

¹H NMR (400 MHz, DMSO-d₆) δ=10.96 (s, 1H) 9.14 (br s, 1H) 8.16 (s, 0.382H) 7.53 (d, J=8.70 Hz, 1H) 7.05-7.11 (m, 5H) 6.93-7.00 (m, 1H) 6.69 (s, 4H) 6.62 (d, J=8.58 Hz, 1H) 6.58 (d, J=2.26 Hz, 1H) 6.49 (dd, J=8.34, 2.50 Hz, 1H) 5.05 (dd, J=13.41, 5.07 Hz, 1H) 4.29-4.37 (m, 1H) 4.17-4.25 (m, 1H) 3.96 (s, 1H) 3.59 (br d, J=10.37 Hz, 2H) 3.29 (br s, 5H) 2.86-2.95 (m, 2H) 2.52-2.68 (m, 7H) 2.27-2.48 (m, 2H) 2.22 (br d, J=7.03 Hz, 2H) 2.03-2.16 (m, 2H) 1.89-1.99 (m, 2H) 1.77-1.87 (m, 3H) 1.67 (br s, 1H) 1.32-1.43 (m, 1H) 1.16-1.26 (m, 2H)

SFC: retention time, 2.834 min; Area, 93.878%;

### Example 181. Preparation of (I-261) (S)-3-(5-(4-((1-(4-((1S*,1′R*)-6′-hydroxy-2,3,3′,4′-tetrahydro-1′H-spirofindene-1,2′-naphthalen]-1′-yl)phenyl)piperidin-4-yl)methyl)piperazin-1-yl)-1-oxoisoindolin-2-yl)piperidine-2,6-dione

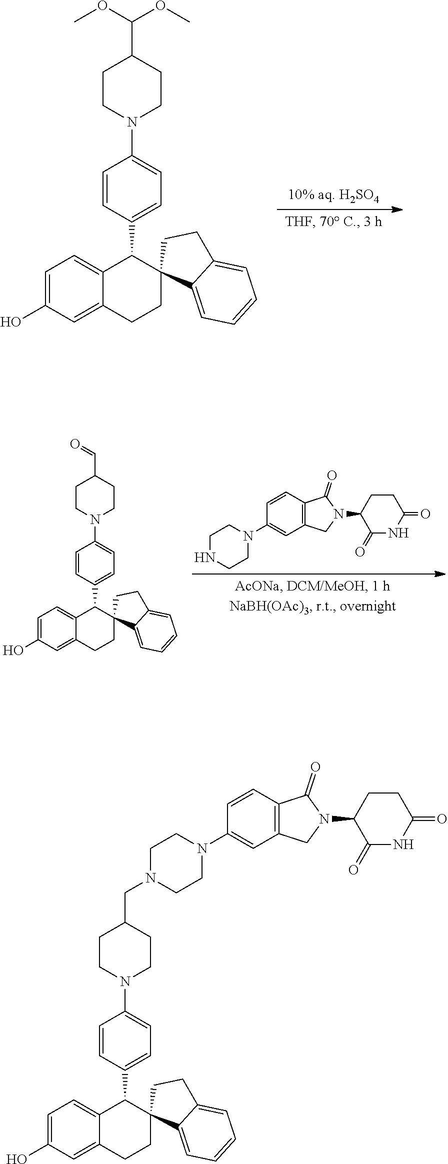 [SMILES: COC(OC)C1CCN(c2ccc([C@H]3c4ccc(O)cc4CC[C@@]34CCc3ccccc34)cc2)CC1 ; O=CC1CCN(c2ccc([C@H]3c4ccc(O)cc4CC[C@@]34CCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@@]78CCc7ccccc78)cc6)CC5)CC4)ccc3C2=O)C(=O)N1]

Step 1:1-(4-((1S,1′R)-6′-hydroxy-2,3,3′,4′-tetrahydro-1′H-spiro[indene-1,2′-naphthalen]-1′-yl)phenyl)piperidine-4-carbaldehyde (105 mg, 236 μmol, 114%, 98.453% purity) in sulfuric acid (202.8 mg, 3 mL, 10% Wt, 1 Eq, 206.8 μmol) and THF (3 mL) for stirred at 70° C. for 16 hour. LCMS showed 98.453% desired MS. The reaction was adjust to pH=˜7 with NaHCO₃, then 50 mL water was added, and the mixture was extracted with dichloromethane (100 mL×2).

The combined organic layers were washed with brine (100 mL), dried over anhydrous Na₂SO₄, filtered and concentrated to dryness in vacuo to give a yellow oil. The yellow oil was concentrated in vacuo to give 1-(4-((1S, 1′R)-6′-hydroxy-2, 3, 3′, 4′-tetrahydro-1′H-spiro[indene-1, 2′-naphthalen]-1′-yl)phenyl)piperidine-4-carbaldehyde (105 mg, 236 μmol, 114%, 98.453% purity) as a yellow solid. LC-MS (ESI+) m/z: 438.1 (M+H)⁺.

Step 2: To a solution of 1-(4-((1S, 1′R)-6′-hydroxy-2,3,3′,4′-tetrahydro-1′H-spirofindene-1,2′-naphthalen]-1′-yl)phenyl)piperidine-4-carbaldehyde (100.0 mg, 1 Eq, 228.5 μmol), (S)-3-(1-oxo-5-(piperazin-1-yl)isoindolin-2-yl)piperidine-2,6-dione, Benzenesulfonic acid salt (137.2 mg, 1.2 Eq. 274.2 μmol) in DCM (2 mL), MeOH (2 mL) was added sodium acetate (56.24 mg, 3 Eq, 685.6 μmol) was stirred at 25° C. for 60 mins, Then sodium triacetoxyborohydride (96.87 mg, 2 Eq, 457.1 μmol) and acetic acid (41.17 mg, 39.43 μL, 3 Eq, 685.6 μmol) was added. The mixture was stirred at 25° C. for 16 hour. LCMS showed 86.788% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 10%-50%,9 min to give (S)-3-(5-(4-((1-(4-((1S,1′R)-6′-hydroxy-2,3,3′,4′-tetrahydro-1′H-spirofindene-1,2′-naphthalen]-1′-yl)phenyl)piperidin-4-yl)methyl)piperazin-1-yl)-1-oxoisoindolin-2-yl)piperidine-2,6-dione (46.6 mg, 62.0 μmol, 27.1%, 99.8% purity) was obtained as a white solid. LC-MS (ESI+) m/z: 750.4 (M+H)⁺.

LCMS: calc. for C₄₇H₅₁N₅O₄, found: [M+H]⁺750.2.

HPLC: 99.8% purity at 220 nm.

¹H NMR (400 MHz, DMSO-d₆) δ=10.96 (s, 1H) 9.11 (br s, 1H) 8.16 (s, 0.530H) 7.53 (d, J=8.70 Hz, 1H) 7.04-7.11 (m, 5H) 6.94-6.99 (m, 1H) 6.69 (s, 4H) 6.62 (d, J=8.46 Hz, 1H) 6.58 (d, J=2.27 Hz, 1H) 6.50 (dd, J=8.34, 2.38 Hz, 1H) 5.06 (dd, J=13.17, 5.07 Hz, 1H) 4.30-4.38 (m, 1H) 4.16-4.25 (m, 1H) 3.95 (s, 1H) 3.51-3.75 (m, 2H) 3.29 (br s, 6H) 2.86-2.95 (m, 2H) 2.55-2.66 (m, 5H) 2.52-2.54 (m, 2H) 2.37 (td, J=13.20, 8.52 Hz, 1H) 2.22 (br d, J=7.03 Hz, 2H) 2.04-2.18 (m, 2H) 1.89-1.99 (m, 2H) 1.76-1.87 (m, 3H) 1.67 (br s, 1H) 1.34-1.42 (m, 1H) 1.15-1.26 (m, 2H).

SFC: retention time, 2.892 min; Arca, 93.884%;

### Example 182. Preparation of (I-260) (S)-3-(5-(4-((1-(4-((1S,1′S)-6′-hydroxy-2,3,3′,4′-tetrahydro-1′H-spirofindene-1,2′-naphthalen]-1′-yl)phenyl)piperidin-4-yl)methyl)piperazin-1-yl)-1-oxoisoindolin-2-yl)piperidine-2,6-dione

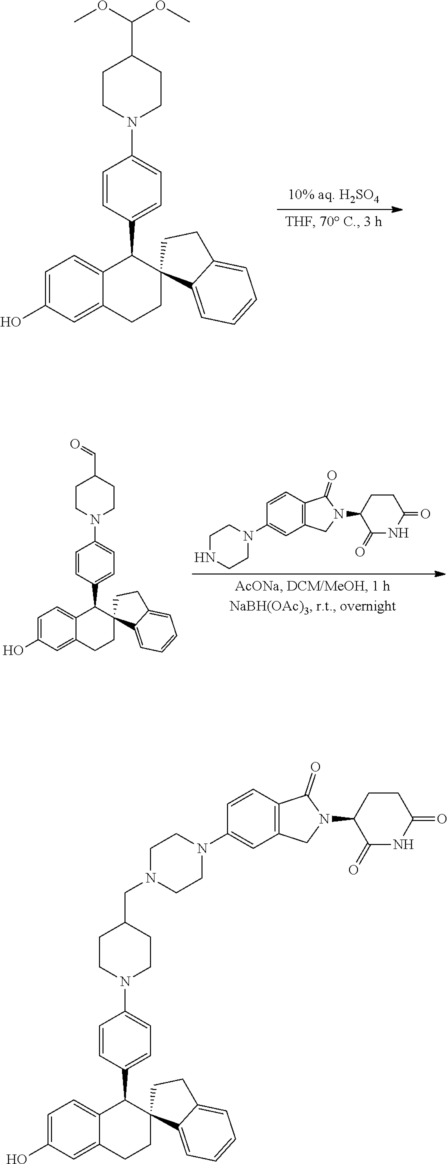 [SMILES: COC(OC)C1CCN(c2ccc([C@@H]3c4ccc(O)cc4CC[C@@]34CCc3ccccc34)cc2)CC1 ; O=CC1CCN(c2ccc([C@@H]3c4ccc(O)cc4CC[C@@]34CCc3ccccc34)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CC[C@@]78CCc7ccccc78)cc6)CC5)CC4)ccc3C2=O)C(=O)N1]

Step 1:1-(4-((1S, 1′S)-6′-hydroxy-2,3,3′,4′-tetrahydro-1′H-spiro[indene-1,2′-naphthalen]-1′-yl)phenyl)piperidine-4-carbaldehyde (130 mg, 293 μmol, 94.4%, 98.506% purity) in sulfuric acid (304.2 mg, 3 mL, 10% Wt, 1 Eq, 310.1 μmol) and THF (3 mL) was stirred at 70° C. for 16 hour. LCMS showed 84.729% desired MS. The reaction was adjust to pH=˜7 with NaHCO₃, then 50 mL water was added, and the mixture was extracted with dichloromethane (100 mL×2). The combined organic layers were washed with brine (100 mL.), dried over anhydrous Na₂SO₄, filtered and concentrated to dryness in vacuum to give a yellow oil. The yellow oil was concentrated in vacuum to give 1-(4-((1S,1′S)-6′-hydroxy-2, 3, 3′, 4′-tetrahydro-1′H-spiro[indene-1, 2′-naphthalen]-1′-yl)phenyl)piperidine-4-carbaldehyde (130 mg, 293 μmol, 94.4%, 98.506% purity) as a yellow solid. LC-MS (ESI+) m/z: 456.3 (M+H)⁺.

Step 2: To a solution of 1-(4-(1S, 1′S)-6′-hydroxy-2,3,3′,4′-tetrahydro-1′H-spiro[indene-1,2′-naphthalen]-1′-yl)phenyl)piperidine-4-carbaldehyde (130.0 mg, 1 Eq, 297.1 μmol), (S)-3-(1-oxo-5-(piperazin-1-yl)isoindolin-2-yl)piperidine-2,6-dione, Benzenesulfonic acid salt (178.4 mg, 1.2 Eq, 356.5 μmol) in DCM (2 mL), MeOH (2 mL) was added sodium acetate (73.11 mg, 3 Eq, 891.3 μmol) was stirred at 25° C. for 60 mines, Then sodium triacetoxyborohydride (125.9 mg, 2 Eq, 594.2 μmol) and acetic acid (53.52 mg, 51.26 μL, 3 Eq, 891.3 μmol) was added The mixture was stirred at 25° C. for 16 hour. LCMS showed 99% desired MS was detected. The reaction mixture was concentrated under reduced pressure to remove solvent. The residue was purified by prep-HPLC (column: Xtimate C18 150*40 mm*5 um phase: [water (FA)-ACN]; B %: 15%-55%, 8 min to give (S)-3-(5-(4-((1-(4-((1S,1′S)-6′-hydroxy-2,3,3′,4′-tetrahydro-1′H-spirofindene-1,2′-naphthalen]-1′-yl)phenyl)piperidin-4-yl)methyl)piperazin-1-yl)-1-oxoisoindolin-2-yl)piperidine-2,6-dione (71.8 mg, 94.2 μmol, 31.7%, 98.4% purity) was obtained as a white solid. LC-MS (ESI+) m/z: 750.4 (M+H)⁺.

LCMS: calc. for C₄₇H₅₁N₅O₄, found: [M+H]⁺750.2.

HPLC: 98.356% purity at 220 nm,

¹H NMR (400 MHz, DMSO-d₆) δ=10.96 (s, 1H) 9.11 (s, 1H) 8.14 (s, 0.201H) 7.53 (d, J=8.82 Hz, 1H) 7.18 (d, J=7.39 Hz, 1H) 7.03-7.10 (m, 3H) 6.77 (t, J=7.51 Hz, 1H) 6.57-6.62 (m, 4H) 6.46 (dd, J=8.34, 2.38 Hz, 1H) 6.29 (br d, J=7.87 Hz, 2H) 5.92 (d, J=7.63 Hz, 1H) 5.05 (dd, J=13.29, 5.07 Hz, 1H) 4.33 (d, J=17.05 Hz, 1H) 4.17-4.26 (m, 1H) 3.80 (s, 1H) 3.56 (br d, J=11.80 Hz, 2H) 3.30 (br s, 6H) 3.00-3.07 (m, 1H) 2.86-2.95 (m, 3H) 2.74-2.81 (m, 1H) 2.53-2.63 (m, 5H) 2.31-2.39 (m, 2H) 2.24 (br d, J=5.13 Hz, 2H) 1.89-2.00 (m, 3H) 1.78 (br d, J=11.56 Hz, 2H) 1.67 (br s, 1H) 1.40-1.48 (m, 1H) 1.16-1.26 (m, 2H)

SFC: retention time, 2.86 min; Area, 91.472%;

### Example 183. Preparation of (I-259) (S)-3-(5-(4-((1-(4-((1R,1′R)-6′-hydroxy-2,3,3′,4′-tetrahydro-1′H-spiro[indene-1,2′-naphthalen]-1′-yl)phenyl)piperidin-4-yl)methyl)piperazin-1-yl)-1-oxoisoindolin-2-yl)piperidine-2,6-dione

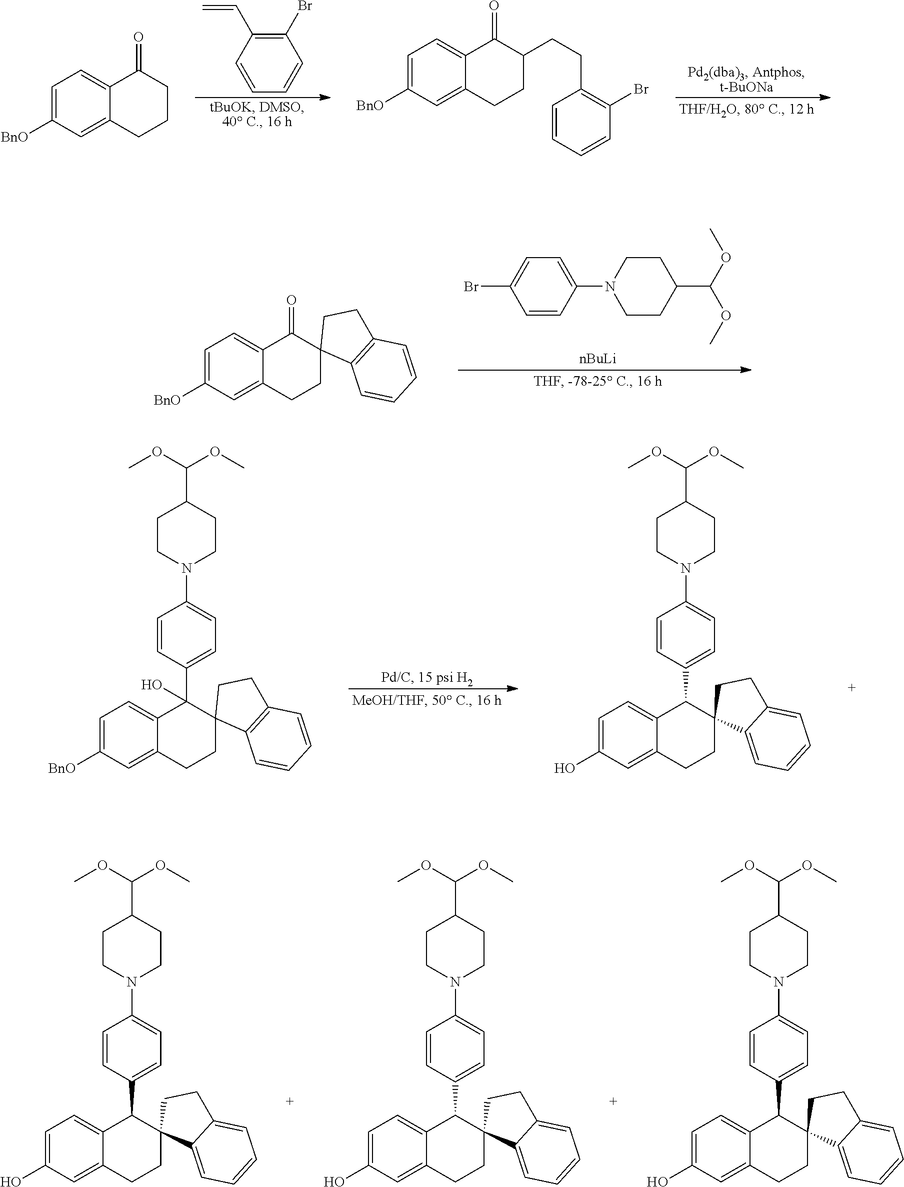

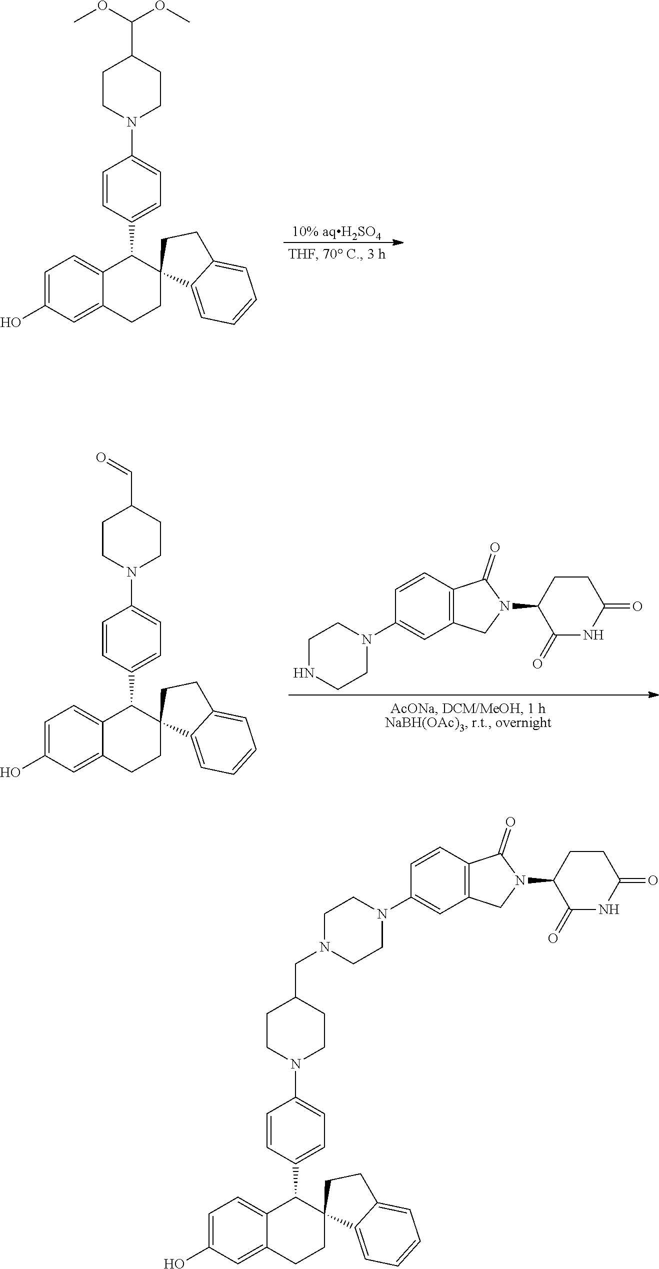

Step 1: To a stirred solution of 6-(benzyloxy)-3,4-dihydronaphthalen-1 (2H)-one (41.4 g, 3 Eq, 164 mmol) in DMSO (50 mL) were added sodium 2-methylpropan-2-olate (2.10 g, 2.36 mL, 0.4 Eq, 21.9 mmol) and 1-bromo-2-vinylbenzene (10.0 g, 6.85 mL, 1 Eq, 54.6 mmol). The reaction mixture was vigorously stirred for 15 h at 40° C. LCMS showed 23.435% desired MS.TLC (petroleum ether:ethyl acetate=5:1, UV) showed one main new spot was observed. 500 mL water was added, and the mixture was extracted with dichloromethane (500 mL×2). The combined organic layers were washed with brine (10 mL), dried over anhydrous Na₂SO₄, filtered and concentrated to dryness in vacuum to give a yellow oil. The yellow oil was purified by flash column (petroleum ether/ethyl acetate from 100/0 to 0/100) and the organic layer was concentrated in vacuum to give 6-(benzyloxy)-2-(2-bromophenethyl)-3, 4-dihydronaphthalen-1 (2H)-one (2 g, 5 mmol, 8%) as a yellow solid. The yellow solid was purified by preparative high-performance liquid chromatography. Condition: Column: Welch Xtimate C18 150*30 mm*5 um A: water (FA) B: ACN at the beginning: A (80%) and B (20%) at the end: A: (0%) and B (100%) Gradient Time (min) 9; 100% B hold Time (min) 2, Flow Rate (ml/min) 30. The pure fractions were collected and the solvent was evaporated under vacuum. The aqueous layer was lyophilized to dryness to give 6-(benzyloxy)-2-(2-bromophenethyl)-3, 4-dihydronaphthalen-1 (2H)-one (2 g, 5 mmol, 8%) as a yellow solid.

Step 2: To a mixture of (RS)-4-(Anthracen-9-yl)-3-(Tert-Butyl)-2,3-Dihydrobenzo[d][1,3]Oxaphosphole (204 mg, 0.12 Eq, 551 μmol), sodium 2-methylpropan-2-olate (1.10 g, 2.5 Eq, 11.5 mmol) and Tris(dibenzylideneacetone)dipalladium (421 mg, 0.1 Eq, 459 μmol) and 6-(benzyloxy)-2-(2-bromophenethyl)-3,4-dihydronaphthalen-1 (2H)-one (2.00 g, 1 Eq, 4.59 mmol) in THF (30 mL) and water (6 mL) was stirred at 70° C. for 16 hour under N₂. LCMS showed 57.487% desired MS. TLC (petroleum ether:ethyl acetate=5:1, UV) showed one main new spot was observed. 100 mL water was added, and the mixture was extracted with dichloromethane (200 mL×2). The combined organic layers were washed with brine (100 mL), dried over anhydrous Na₂SO₄, filtered and concentrated to dryness in vacuum to give a yellow oil. The yellow oil was purified by flash column (petroleum ether/ethyl acetate from 100/0 to 0/100) and the organic layer was concentrated in vacuum to give 6′-(benzyloxy)-2,3,3′,4′-tetrahydro-1′H-spirofindene-1,2′-naphthalen]-1′-one (1.5 g, 3.0 mmol, 66%, 71.352% purity) as a yellow solid. LC-MS (ESI+) m/z: 335.2 (M+H)⁺.

Step 3: To a solution of 1-(4-bromophenyl)-4-(dimethoxymethyl)piperidine (2.00 g, 1 Eq, 6.36 mmol) in THF (50 mL) was added n-Butyl lithium, 2.5M in hexane (408 mg, 2.55 mL, 2.5 molar, 1 Eq, 6.36 mmol) at −68° C. under N₂ over 5 min. After addition, the mixture was stirred at −68° C. for 1 hour and then 6′-(benzyloxy)-2, 3, 3′, 4′-tetrahydro-1′H-spiro[indene-1, 2′-naphthalen]-1′-one (1.42 g, 0.45 Eq, 2.86 mmol) was added dropwise at −68° C. The resulting mixture was stirred at 25° C. for 16 hour. LCMS showed 48.689% desired MS. The reaction was quenched with water (200 mL) and extracted with ethyl acetate (200 mL*2). The organic layer was washed with brine (100 mL) and dried over Na₂SO₄ and concentrated in vacuum to give a yellow oil. The white solid was subjected to column chromatography over silica gel (20 g gradient elution: 0-30% EtOAc). The desired fractions were collected, and concentrated to dryness in vacuum to give 6′-(benzyloxy)-1′-(4-(4-(dimethoxymethyl)piperidin-1-yl)phenyl)-2,3,3′,4′-tetrahydro-1′H-spiro[indene-1,2′-naphthalen]-1′-ol (400 mg, 0.66 mmol, 10%, 97% purity) and 6′-(benzyloxy)-2,3,3′,4′-tetrahydro-1′H-spiro[indene-1,2′-naphthalen]-1′-one (800 mg, 2.26 mmol, 35.5%) as a yellow solid. LC-MS (ESI+) m/z: 572.3 (M+H)⁺.

Step 4: To a solution of 6′-(benzyloxy)-1′-(4-(4-(dimethoxymethyl)piperidin-1-yl)phenyl)-2,3,3′,4′-tetrahydro-1′H-spiro[indene-1,2′-naphthalen]-1′-ol (1.00 g, 1 Eq, 1.39 mmol) in MeOH (20 mL) and THF (20 mL) was added Pd/C (1.48 g, 10% Wt, 1 Eq, 1.39 mmol) under N₂ atmosphere. The suspension was degassed and purged with dihydrogen for 3 mins. The mixture was stirred under 15 psi dihydrogen (2.81 mg, 1 Eq, 1.39 mmol) at 50° C. for 16 hr. LCMS showed 74.8% desired MS was detected. The mixture was filtered and concentrated under reduced pressure to give 1′-(4-(4-(dimethoxymethyl)piperidin-1-yl)phenyl)-2,3,3′,4′-tetrahydro-1′H-spirofindene-1,2′-naphthalen]-6′-ol (0.8 g, 2 mmol, 100%) as a yellow solid. The compounds were concentrated under reduced pressure to give desired compound as a yellow oil, which was further separated by SFC (condition: column: DAICEL CHIRALCEL OJ (250 mm*30 mm, 10 um)); mobile phase: [0.1% NH₃·H₂O EtOH]; B %: 35%-35%) to give (1R, 1′R)-1′-(4-(4-(dimethoxymethyl)piperidin-1-yl)phenyl)-2,3,3′,4′-tetrahydro-1′H-spiro[indene-1,2′-naphthalen]-6′-ol (0.3 g, 0.6 mmol,) and (1S, 1′S)-1′-(4-(4-(dimethoxymethyl)piperidin-1-yl)phenyl)-2,3,3′,4′-tetrahydro-1′H-spiro[indene-1,2′-naphthalen]-6′-ol (0.3 g, 0.6 mmol,) and (1S, 1′R)-1′-(4-(4-(dimethoxymethyl)piperidin-1-yl)phenyl)-2,3,3′,4′-tetrahydro-1′H-spiro[indene-1,2′-naphthalen]-6′-ol (0.145 g, 300 μmol,) and (1R, 1′S)-1′-(4-(4-(dimethoxymethyl)piperidin-1-yl)phenyl)-2,3,3′,4′-tetrahydro-1′H-spiro[indene-1,2′-naphthalen]-6′-ol (0.14 g, 0.29 mmol, 21%) as a white solid. LC-MS (ESI+) m/z: 484.3 (M+H)⁺.

Step 5: (1R, 1′R)-1′-(4-(4-(dimethoxymethyl)piperidin-1-yl)phenyl)-2,3,3′,4′-tetrahydro-1′H-spiro[indene-1,2′-naphthalen]-6′-ol (150.0 mg, 1 Eq, 310.1 μmol) in sulfuric acid (304.2 mg, 3 mL, 10% Wt, 1 Eq, 310.1 μmol) and THE (3 mL) for stirred at 70° C. for 16 hour. LCMS showed 96.995% desired MS. The reaction was adjust to pH=˜7 with NaHCO₃, then 50 mL water was added, and the mixture was extracted with dichloromethane (100 mL×2). The combined organic layers were washed with brine (100 mL), dried over anhydrous Na₂SO₄, filtered and concentrated to dryness in vacuo to give a yellow oil. The yellow oil was concentrated in vacuo to give 1-(4-((1R, 1′R)-6′-hydroxy-2, 3, 3′, 4′-tetrahydro-1′H-spiro[indene-1, 2′-naphthalen]-1′-yl)phenyl)piperidine-4-carbaldehyde (110 mg, 244 μmol, 78.6%, 96.995% purity) as a yellow solid. LC-MS (ESI+) m/z: 456.3 (M+H)⁺.

Step 6: To a solution of 1-(4-((1R, 1′R)-6′-hydroxy-2,3,3′,4′-tetrahydro-1′H-spiro[indene-1,2′-naphthalen]-1′-yl)phenyl)piperidine-4-carbaldehyde (180.0 mg, 1 Eq, 411.4 μmol), (S)-3-(1-oxo-5-(piperazin-1-yl)isoindolin-2-yl)piperidine-2,6-dione, Benzenesulfonic acid salt (247.0 mg, 1.2 Eq, 493.6 μmol) in DCM (2 mL), MeOH (2 mL) was added sodium acetate (101.2 mg, 3 Eq, 1.234 mmol) was stirred at 25° C. for 60 mins, Then sodium triacetoxyborohydride (174.4 mg, 2 Eq. 822.7 μmol) and acetic acid (74.11 mg, 70.98 μL, 3 Eq, 1.234 mmol) was added The mixture was stirred at 25° C. for 16 hour. LCMS showed 85.6% desired MS was detected. The reaction mixture was concentrated under reduced pressure to remove solvent. The residue was purified by prep-HPLC (column: Welch Xtimate C18 150*30 mm*5 um phase: [water (FA)-ACN]; B %: 15%-55%,9 min to give (S)-3-(5-(4-((1-(4-((1R, 1′R)-6′-hydroxy-2,3,3′,4′-tetrahydro-1′H-spirofindene-1,2′-naphthalen]-1′-yl)phenyl)piperidin-4-yl)methyl)piperazin-1-yl)-1-oxoisoindolin-2-yl)piperidine-2,6-dione (54.3 mg, 70.9 μmol, 17.2%, 97.9% purity) was obtained as a white solid. LC-MS (ESI+) m/z: 750.4 (M+H)⁺.

LCMS: calc. for C₄₇H₅₁N₅O₄, found: [M+H]⁺750.2.

HPLC: 97.9% purity at 220 nm.

¹H NMR (400 MHz, DMSO-d₆) δ=10.96 (s, 1H) 9.11 (s, 1H) 8.14 (s, 0.214H) 7.53 (d, J=8.46 Hz, 1H) 7.18 (d, J=7.51 Hz, 1H) 7.03-7.09 (m, 3H) 6.77 (t, J=7.51 Hz, 1H) 6.57-6.62 (m, 4H) 6.46 (dd, J=8.34, 2.38 Hz, 1H) 6.29 (br d, J=7.87 Hz, 2H) 5.92 (d, J=7.51 Hz, 1H) 5.05 (dd, J=13.23, 5.13 Hz, 1H) 4.33 (d, J=17.05 Hz, 1H) 4.17-4.25 (m, 1H) 3.80 (s, 1H) 3.51-3.63 (m, 2H) 3.23-3.32 (m, 6H) 2.84-3.08 (m, 5H) 2.75-2.81 (m, 1H) 2.55-2.62 (m, 4H) 2.37 (br dd, J=13.29, 4.59 Hz, 2H) 2.26 (br s, 2H) 1.90-2.00 (m, 3H) 1.78 (br d, J=12.04 Hz, 2H) 1.68 (br s, 1H) 1.44 (br d, J=12.76 Hz, 1H) 1.16-1.26 (m, 2H).

SFC: retention time, 2.842 min; Arca, 92.049%;

### Example 184. Preparation of (I-240) (S)-3-(5-(4-((1-(4-((1R,2S)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione

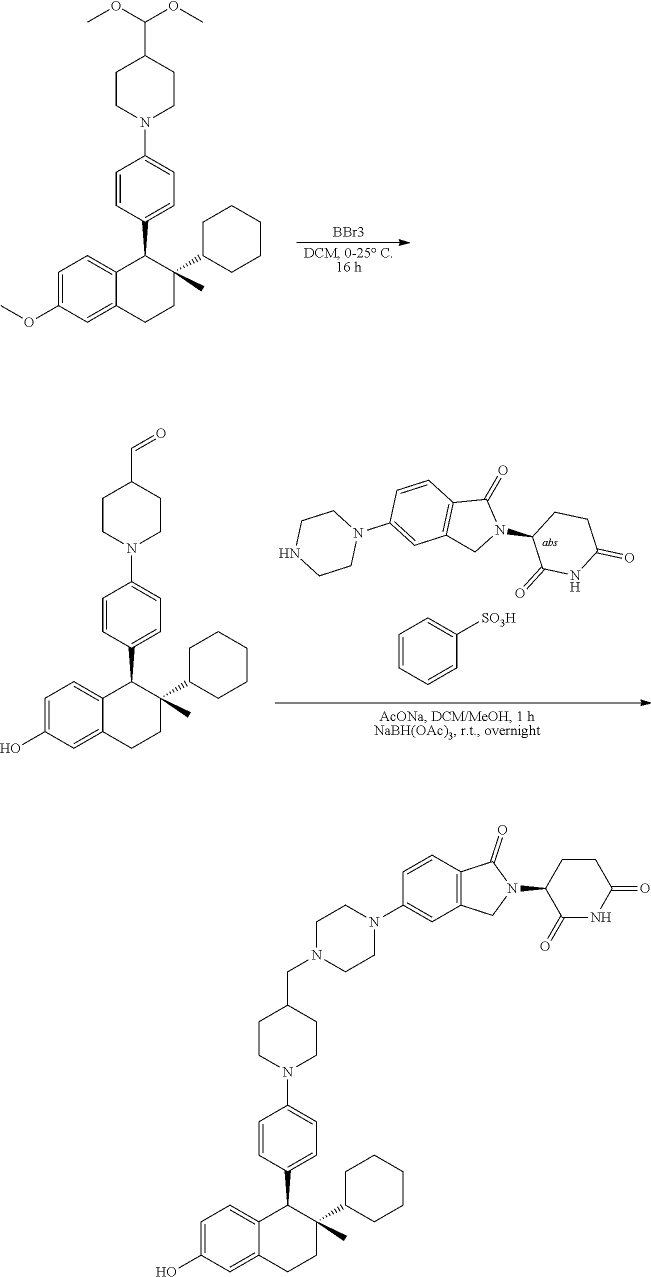

Step 1: To a solution of 1-(4-(1R,2S)-2-cyclohexyl-6-methoxy-2-methyl-1,2,3,4-tetrahydronaphthalen-1-yl)phenyl)-4-(dimethoxymethyl)piperidine (80 mg, 1 Eq. 0.16 mmol) in DCM (2 mL) at 0° C. was added tribromoborane (0.12 g, 0.48 mL, 1 M, 3 Eq, 0.48 mmol) with N₂. After addition, the mixture was stirred at 0° C. for 4 hour, and then the reaction was warmed to 25° C. and was stirred at this temperature for 12 hour. The reaction mixture was quenched with saturated 10 mL NaHCO₃ and stirred for 10 min at 0° C. Then the mixture was dissolved in water (30 mL) and extracted by ethyl acetate (40 mL). The combined organic layer was dried over Na₂SO₄, filtered and concentrated under vacuum to give the compound 1-(4-((1R,2S)-2-cyclohexyl-6-hydroxy-2-methyl-1,2,3,4-tetrahydronaphthalen-1-yl)phenyl)piperidine-4-carbaldehyde (70 mg, 0.16 mmol, 100%) as a yellow solid. LC-MS (ESI+) m/z: 432.1 (M+H)+.

Step 2: A mixture of 1-(4-((1R,2S)-2-cyclohexyl-6-hydroxy-2-methyl-1,2,3,4-tetrahydronaphthalen-1-yl)phenyl)piperidine-4-carbaldehyde (60 mg, 1 Eq, 0.14 mmol), (S)-3-(1-oxo-5-(piperazin-1-yl)isoindolin-2-yl)piperidine-2,6-dione benzenesulfonate (88 mg, 1.3 Eq, 0.18 mmol) and sodium acetate (57 mg, 5 Eq, 0.70 mmol) and acetic acid (25 mg, 24 μL, 3 Eq, 0.42 mmol) in DCM (2 mL) and MeOH (2 mL) at 25° C. for 1 hour, then sodium triacetoxyborohydride (59 mg, 2 Eq, 0.28 mmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*40 mm*5 um mobile phase: [water (FA)-ACN]; B %: 20%-60%, 9 min). The pure fractions were collected respectively and the solvents were evaporated under vacuum to give the product (S)-3-(5-(4-((1-(4-((1R,2S)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione (34 mg, 46 μmol, 33%, 99.674% purity) as a white solid.

LC-MS (ESI+) m/z: 744.5 (M+H)⁺.

HPLC: 99.674%, purity at 220 nm.

SFC: retention time, 3.017 min; Arca, 81.989%;

¹H NMR (400 MHZ, DMSO-d₆) δ=10.96 (s, 1H), 9.17-8.90 (m, 1H), 8.17 (s, 0.420H), 7.53 (d, J=8.6 Hz, 1H), 7.11-7.02 (m, 2H), 6.76 (s, 4H), 6.60-6.48 (m, 2H), 6.41 (dd, J=2.3, 8.3 Hz, 1H), 5.06 (dd, J=5.0, 13.2 Hz, 1H), 4.40-4.29 (m, 1H), 4.26-4.15 (m, 1H), 3.89 (s, 1H), 3.65-3.57 (m, 2H), 3.29 (br s, 5H), 2.98-2.84 (m, 1H), 2.73-2.56 (m, 6H), 2.40-2.29 (m, 1H), 2.22 (br d, J=6.9 Hz, 2H), 2.01-1.92 (m, 1H), 1.89-1.40 (m, 11H), 1.36-0.84 (m, 9H), 0.49 (s, 3H).

### Example 185. Preparation of (I-239) (S)-3-(5-(4-((1-(4-((1S,2R)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione

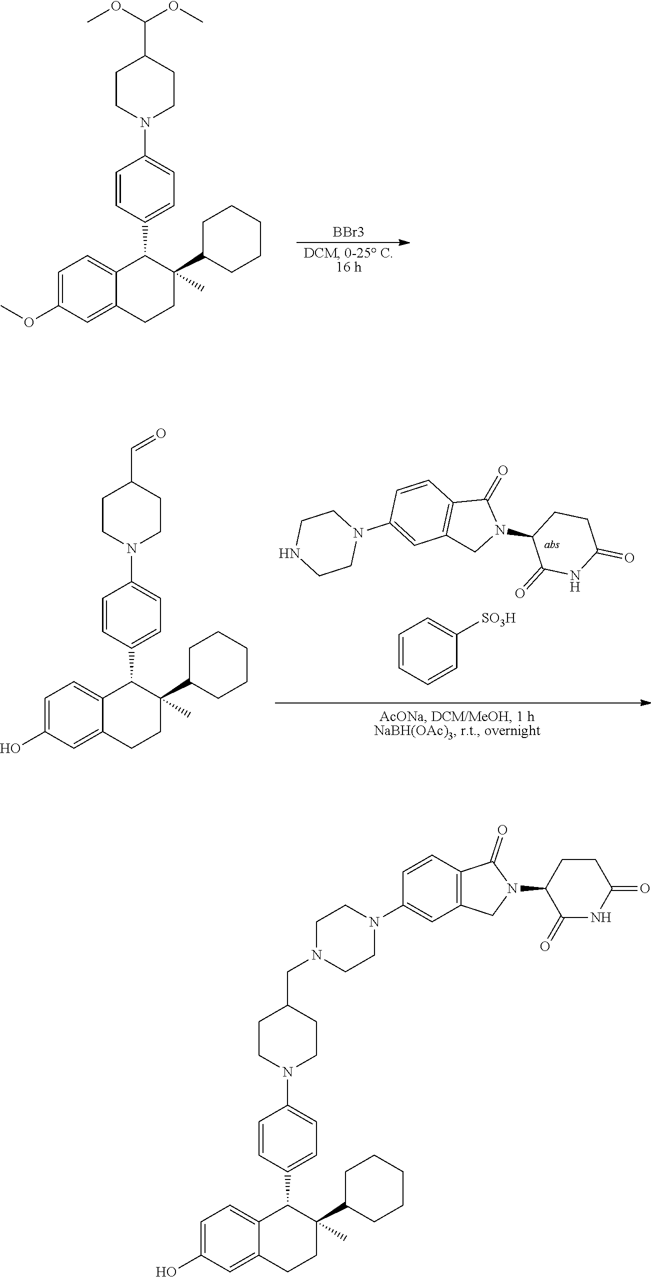

Step 1: To a solution of 1-(4-((1S,2R)-2-cyclohexyl-6-methoxy-2-methyl-1,2,3,4-tetrahydronaphthalen-1-yl)phenyl)-4-(dimethoxymethyl)piperidine (80 mg, 1 Eq, 0.16 mmol) in DCM (2 mL) at 0° C. was added tribromoborane (0.12 g, 0.49 mL, 1 M, 3 Eq, 0.49 mmol) with N₂. After addition, the mixture was stirred at 0° C. for 4 hour, and then the reaction was warmed to 25° C. and was stirred at this temperature for 12 hour. The reaction mixture was quenched with saturated 10 mL NaHCO₃ and stirred for 10 min at 0° C. Then the mixture was dissolved in water (30 mL) and extracted by ethyl acetate (40 mL). The combined organic layer was dried over Na₂SO₄, filtered and concentrated under vacuum to give the compound 1-(4-((1S,2R)-2-cyclohexyl-6-hydroxy-2-methyl-1,2,3,4-tetrahydronaphthalen-1-yl)phenyl)piperidine-4-carbaldehyde (70 mg, 0.16 mmol, 100%) as a yellow solid. LC-MS (ESI+) m/z: 432.2 (M+H)+.

Step 2: A mixture of 1-(4-((1S,2R)-2-cyclohexyl-6-hydroxy-2-methyl-1,2,3,4-tetrahydronaphthalen-1-yl)phenyl)piperidine-4-carbaldehyde (60 mg, 1 Eq, 0.14 mmol), (S)-3-(1-oxo-5-(piperazin-1-yl)isoindolin-2-yl)piperidine-2,6-dione benzenesulfonate (88 mg, 1.3 Eq, 0.18 mmol) and sodium acetate (57 mg, 5 Eq, 0.70 mmol) and acetic acid (25 mg, 24 μL, 3 Eq, 0.42 mmol) in DCM (2 mL) and MeOH (2 mL) at 25° C. for 1 hour, then sodium triacetoxyborohydride (59 mg, 2 Eq, 0.28 mmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*40 mm*3 um mobile phase: [water (FA)-ACN]; B %: 20%-60%, 9 min). The aqueous phase was lyophilized to dryness to give (S)-3-(5-(4-((1-(4-((1S,2R)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione (43.4 mg, 58.1 μmol, 42%, 99.547% Purity) as a pink solid.

LC-MS (ESI+) m/z: 744.5 (M+H)+.

HPLC: 99.547%, purity at 220 nm.

SFC: retention time, 2.974 min; Area, 87.861%;

¹H NMR (400 MHz, DMSO-d₆) δ=10.96 (s, 1H), 9.19-8.88 (m, 1H), 8.16 (s, 0.611H), 7.53 (d, J=8.6 Hz, 1H), 7.11-7.03 (m, 2H), 6.82-6.72 (m, 4H), 6.58-6.49 (m, 2H), 6.41 (dd, J=2.4, 8.3 Hz, 1H), 5.06 (dd, J=5.1, 13.2 Hz, 1H), 4.39-4.29 (m, 1H), 4.26-4.16 (m, 1H), 3.89 (s, 1H), 3.65-3.57 (m, 2H), 3.29 (br s, 5H), 2.99-2.84 (m, 1H), 2.72-2.56 (m, 6H), 2.39-2.33 (m, 1H), 2.22 (br d, J=7.2 Hz, 2H), 2.01-1.92 (m, 1H), 1.90-1.41 (m, 11H), 1.29-0.86 (m, 9H), 0.49 (s, 3H).

### Example 186. Preparation of (I-238) (S)-3-(5-(4-((1-(4-((1R,2R)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione

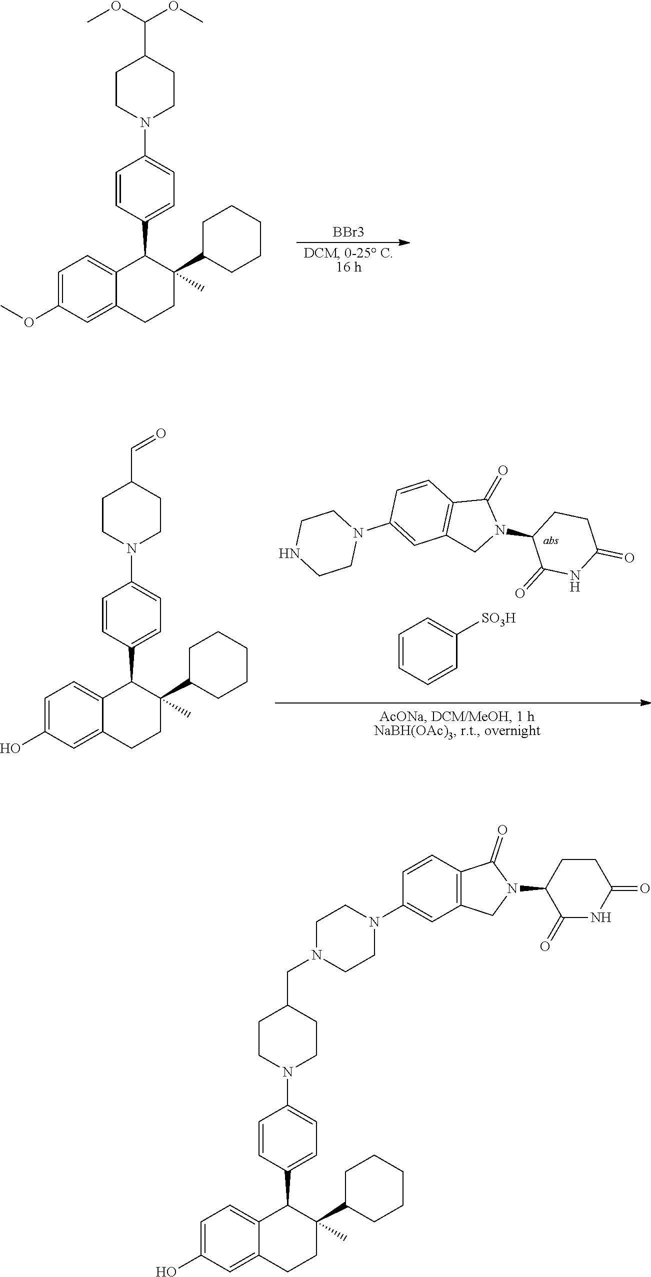

Step 1: To a solution of 1-(4-((1R,2R)-2-cyclohexyl-6-methoxy-2-methyl-1,2,3,4-tetrahydronaphthalen-1-yl)phenyl)-4-(dimethoxymethyl)piperidine (150 mg, 1 Eq, 305 μmol) in DCM (2 mL) at 0° C. was added tribromoborane (229 mg, 915 μL, 1 M, 3 Eq, 915 μmol) with N₂. After addition, the mixture was stirred at 0° C. for 4 hour, then the reaction was warmed to 25° C. and was stirred at this temperature for 12 hour. The reaction mixture was quenched with saturated 10 mL NaHCO₃ and stirred for 10 min at 0° C. Then the mixture was dissolved in water (30 mL) and extracted by ethyl acetate (40 mL). The combined organic layer was dried over Na₂SO₄, filtered and concentrated under vacuum to give the compound 1-(4-((1R,2R)-2-cyclohexyl-6-hydroxy-2-methyl-1,2,3,4-tetrahydronaphthalen-1-yl)phenyl)piperidine-4-carbaldehyde (125 mg, 290 μmol, 94.9%) as a yellow solid. LC-MS (ESI+) m/z: 432.2 (M+H)+.

Step 2: A mixture of 1-(4-((1R,2R)-2-cyclohexyl-6-hydroxy-2-methyl-1,2,3,4-tetrahydronaphthalen-1-yl)phenyl)piperidine-4-carbaldehyde (125 mg, 1 Eq, 290 μmol), (S)-3-(1-oxo-5-(piperazin-1-yl)isoindolin-2-yl)piperidine-2,6-dione benzenesulfonate (183 mg, 1.3 Eq, 376 μmol) and sodium acetate (119 mg, 5 Eq, 1.45 mmol) and acetic acid (52.2 mg, 50.0 μL, 3 Eq. 869 μmol) in DCM (2 mL) and MeOH (2 mL) at 25° C. for 1 hour, then sodium triacetoxyborohydride (123 mg, 2 Eq, 579 μmol) was added to the mixture and stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*30 mm*5 um mobile phase: [water (FA)-ACN]; B %: 20%-60%, 9 min). The aqueous phase was lyophilized to dryness to give (S)-3-(5-(4-((1-(4-((1R,2R)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione (40.7 mg, 54.3 μmol, 18.7%, 99.209% Purity) as a white solid.

LC-MS (ESI+) m/z: 744.5 (M+H)+.

HPLC: 99.209%, purity at 220 nm.

SFC: retention time, 3.053 min; Arca, 75.875%;

¹H NMR (400 MHZ, DMSO-d₆) δ=10.96 (s, 1H), 9.04-8.92 (m, 1H), 8.17 (s, 0.275H), 7.52 (d, J=8.6 Hz, 1H), 7.10-7.03 (m, 2H), 6.84-6.78 (m, 2H), 6.77-6.72 (m, 2H), 6.62 (d, J=8.3 Hz, 1H), 6.51 (d, J=2.3 Hz, 1H), 6.39 (dd, J=2.4, 8.2 Hz, 1H), 5.05 (dd, J=5.1, 13.2 Hz, 1H), 4.38-4.29 (m, 1H), 4.27-4.17 (m, 1H), 3.73 (s, 1H), 3.66-3.56 (m, 2H), 3.31-3.18 (m, 6H), 2.97-2.83 (m, 2H), 2.82-2.71 (m, 1H), 2.65-2.54 (m, 5H), 2.39-2.32 (m, 1H), 2.21 (br d, J=7.2 Hz, 2H), 2.13 (brd, J=11.1 Hz, 1H), 2.01-1.92 (m, 1H), 1.80 (br d, J=11.1 Hz, 2H), 1.74-1.63 (m, 3H), 1.62-1.52 (m, 4H), 1.26-1.15 (m, 2H), 1.09-0.87 (m, 5H), 0.76 (s, 4H).

### Example 187. Preparation of (I-237) (S)-3-(5-(4-((1-(4-((1S,2S)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione

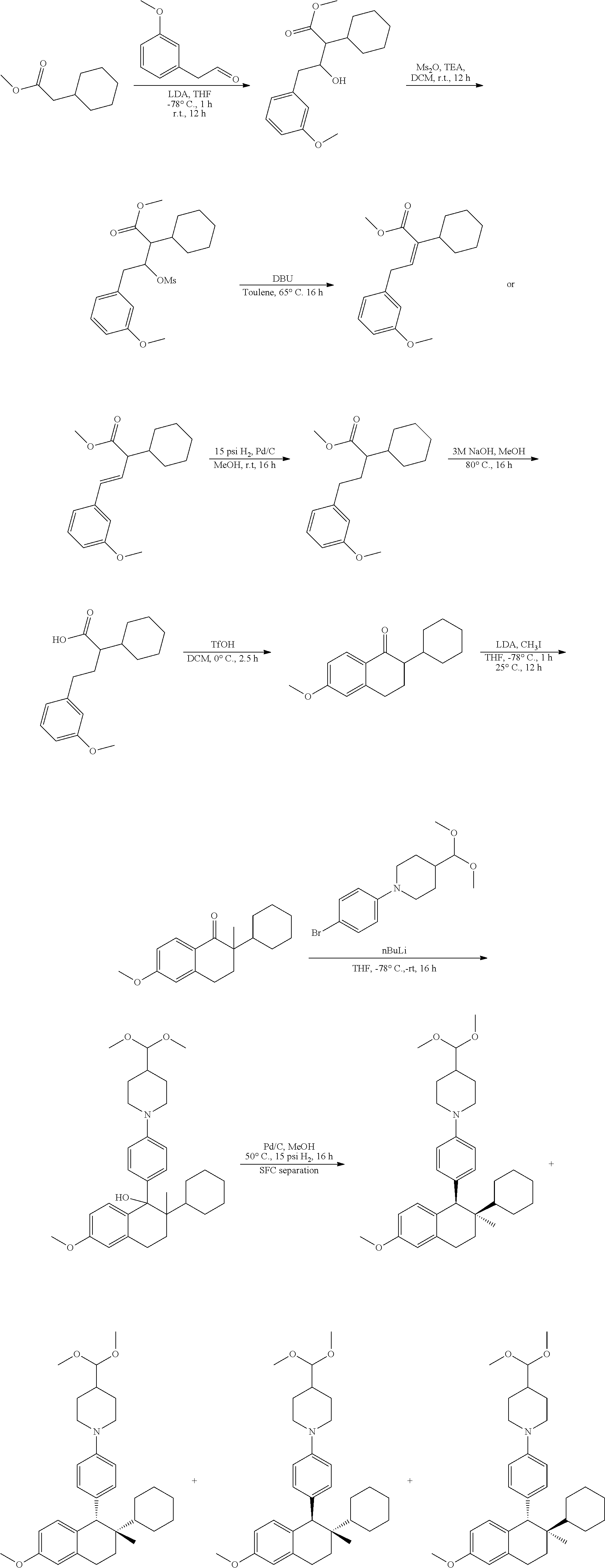

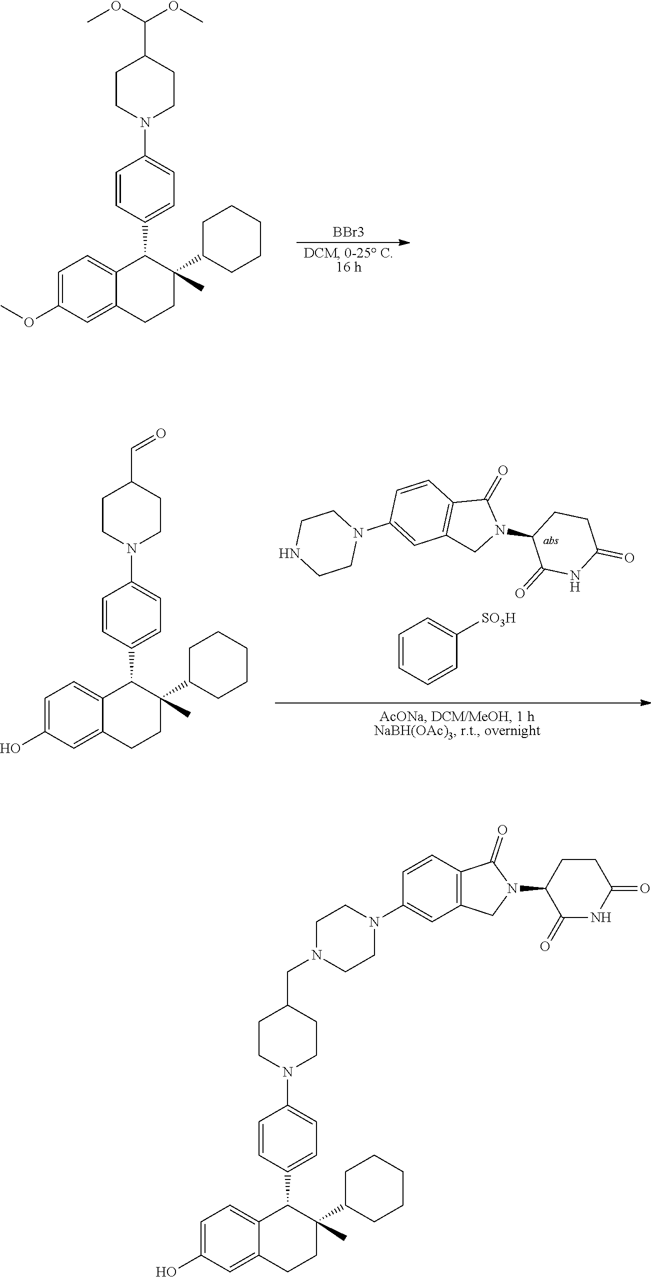

Step 1: To a solution of methyl 2-cyclohexylacetate (100 mg, 105 μL, 1 Eq, 640 μmol) in THE (2 mL) cooled at −78° C. under N₂, lithium diisopropylamide (137 mg, 640 μL, 2 M, 2 Eq, 1.28 mmol) was added to the reaction, stirred at −78° C. for 1 hour, was added dropwise 2-(3-methoxyphenyl) acetaldehyde (144 mg, 0.14 mL, 1.5 Eq, 960 μmol) over 10 min. The reaction mixture was stirred at 25° C. for 12 hour. Then the mixture was cooled to room temperature. The reaction was quenched with NH₄Cl (20 mL), the mixture was treated with H₂O (30 mL), extracted with EtOAc (40 mL). The combined extracts was dried over anhydrous Na₂SO₄, filtered and concentrated to dryness in vacuo to give a yellow oil. The yellow oil was purified by flash column (petroleum ether/ethyl acetate from 100/0 to 90/10) and the organic layer was concentrated in vacuo to give methyl 2-cyclohexyl-3-hydroxy-4-(3-methoxyphenyl) butanoate (100 mg, 326 μmol, 51.0%) as a yellow oil.

¹H NMR (400 MHZ, CD₃Cl) δ=7.19-7.12 (m, 1H), 6.76-6.67 (m, 3H), 4.13-3.93 (m, 1H), 3.78-3.63 (m, 6H), 2.83-2.68 (m, 1H), 2.64-2.54 (m, 1H), 2.47-2.36 (m, 1H), 1.90-1.75 (m, 2H), 1.73-1.50 (m, 5H), 1.29-0.83 (m, 5H).

Step 2: A mixture of Triethylamine (1 g, 1 mL, 3 Eq, 0.01 mol), methanesulfonic anhydride (1 g, 2 Eq, 7 mmol), methyl 2-cyclohexyl-3-hydroxy-4-(3-methoxyphenyl) butanoate (1 g, 1 Eq, 3 mmol) in DCM (10 mL) under N₂. Then the reaction was stirred at 25° C. for 12 hour. The mixture was treated with H₂O (100 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜10% ethyl acetate in petroleum ether). The pure fractions were collected and concentrated to dryness in vacuo to methyl 2-cyclohexyl-4-(3-methoxyphenyl)-3-((methylsulfonyl)oxy) butanoate (700 mg, 1.6 mmol, 50%, 88% purity) as a yellow oil.

¹H NMR (400 MHZ, CD₃Cl) δ=7.23 (d, J=8.3 Hz, 1H), 6.85-6.79 (m, 3H), 5.07 (ddd, J=3.8, 6.3, 9.5 Hz, 1H), 3.81 (s, 3H), 3.76 (s, 3H), 3.12-2.98 (m, 2H), 2.83 (dd, J=6.8, 8.0 Hz, 1H), 2.35 (s, 3H), 1.85-1.63 (m, 8H), 1.27-1.18 (m, 3H).

Step 3: A mixture of methyl 2-cyclohexyl-4-(3-methoxyphenyl)-3-((methylsulfonyl)oxy) butanoate (700 mg, 1 Eq, 1.60 mmol), DBU (732 mg, 724 μL, 3 Eq, 4.81 mmol) in toluene (10 mL) under N₂. Then the reaction was stirred at 65° C. for 16 hour. Then the reaction was cooled to room temperature. To the mixture was added water (100 mL), then was extracted with ethyl acetate 100 mL (50 mL*2). The combined organic layers were washed with brine (50 mL), dried over Na₂SO₄, filtered and concentrated under reduced pressure to give the crude product. The desired product was purified by flash column (petroleum ether/ethyl acetate from 100/0 to 90/10) and the organic layer was concentrated in vacuo to give methyl 2-cyclohexyl-4-(3-methoxyphenyl) but-3-enoate (400 mg, 1.39 mmol, 86.6%, 100% purity) as a yellow oil. LC-MS (ESI⁺) m/z: 289.1 (M+H)⁺.

Step 4: A mixture of methyl 2-cyclohexyl-4-(3-methoxyphenyl) but-3-enoate (400 mg, 1 Eq, 1.39 mmol), Pd/C (600 mg, 10% Wt, 0.406 Eq, 564 μmol), H₂ (2.80 mg, 1 Eq, 1.39 mmol) in MeOH (20 mL). The suspension was degassed and purged with H₂ for 3 times. The mixture was stirred under H₂ (15 psi) at 25° C. for 16 hour. Then the reaction was cooled to room temperature. The mixture was filtered and concentrated to dryness in vacuo to give methyl 2-cyclohexyl-4-(3-methoxyphenyl) butanoate (350 mg, 1.21 mmol, 86.9%, 100% Purity) as a yellow oil. LC-MS (ESI⁺) m/z: 308.1 (M+H₂O)⁺.

Step 5: A 100 mL thread vial equipped with magnetic stirrer, sodium hydroxide (3.6 g, 30 mL, 3 molar, 8.2 Eq, 90 mmol) was added to the solution of methyl 2-cyclohexyl-4-(3-methoxyphenyl) butanoate (3.2 g, 100% Wt, 1 Eq, 11 mmol) in MeOH (15 mL). The mixture was stirred at 80° C. for 16 hour. The reaction was treated with 1 N HCl (100 mL), extracted with EtOAC (100 mL). The combined extracts was dried over anhydrous Na₂SO₄, filtered and concentrated to dryness in vacuo to give 2-cyclohexyl-4-(3-methoxyphenyl) butanoic acid (3 g, 0.01 mol, 100%) as a white solid.

Step 6: A 100 mL thread vial was equipped with magnetic stirrer. Triflic acid (28 g, 17 mL, 20 Eq, 0.19 mol) was added to the solution of 2-cyclohexyl-4-(3-methoxyphenyl) butanoic acid (2.6 g, 1 Eq, 9.4 mmol) in DCM (30 mL). The mixture was stirred at 0° C. for 2.5 hour. The mixture was treated with H₂O (100 mL), extracted with DCM (50 mL*2). The combined extracts was dried over anhydrous Na₂SO₄, filtered and concentrated to dryness in vacuo to give a residue. The residue was subjected to column chromatography over silica gel (gradient elution: 0˜30% ethyl acetate in petroleum ether). The pure fractions were collected and concentrated to dryness in vacuo to give 2-cyclohexyl-6-methoxy-3,4-dihydronaphthalen-1 (2H)-one (2.1 g, 8.1 mmol, 86%, 100% purity) as a yellow solid. LC-MS (ESI⁺) m/z: 259.1 (M+H)⁺.

Step 7: To a solution of 2-cyclohexyl-6-methoxy-3,4-dihydronaphthalen-1 (2H)-one (2 g, 1 Eq, 8 mmol) in THF (15 Ml) was added LDA solution (2 g, 0.01 L, 2 M, 3 Eq, 0.02 mol) at −78° C. The reaction mixture was stirred at −78° C. for 1 hour. Then Methyl iodide (7 g, 3 MI, 6 Eq, 0.05 mol) was added to the mixture dropwise at −78° C. The reaction mixture was warmed to 25° C. and stirred for 12 hour. The mixture was quenched by the addition of 25 MI of saturated aqueous NH₄Cl. The mixture was treated with H₂O (100 Ml), extracted with EtOAc (50 Ml*2). The combined extracts was dried over anhydrous Na₂SO₄, filtered and concentrated to dryness in vacuo to give 2-cyclohexyl-6-methoxy-2-methyl-3,4-dihydronaphthalen-1 (2H)-one (2.1 g, 7.7 mmol, 100%) as a yellow oil. LC-MS (ESI⁺) m/z: 273.2 (M+H)⁺.

Step 8: To a mixture of 1-(4-bromophenyl)-4-(dimethoxymethyl)piperidine (2 g, 2 Eq, 7 mmol) in THF (15 mL) at r.t. and the mixture was degassed and purged with N₂ for 10 minutes. Then the mixture was stirred until it reached −78° C. n-Butyllithium, 2.5 M in hexane (0.4 g, 3 mL, 2.5 M, 1.8 Eq. 7 mmol) was added to the mixture and stirred at −78° C. for 1 hour, then 2-cyclohexyl-6-methoxy-2-methyl-3,4-dihydronaphthalen-1 (2H)-one (1 g, 1 Eq, 4 mmol) was added to the mixture and stirred at 25° C. for 16 hour. TLC (PE/EtOAc=5/1, R_{f}=0.3) showed a new spot was formed. The reaction was quenched with water (100 mL) and extracted with ethyl acetate (100 mL). The organic layer was washed with brine (50 mL) and dried over Na₂SO₄ and concentrated in vacuo to give a yellow oil. The yellow oil was subjected to column chromatography over silica gel (gradient elution: 0-40% EtOAc). The desired fractions were collected, and concentrated to dryness in vacuo to give 2-cyclohexyl-1-(4-(4-(dimethoxymethyl)piperidin-1-yl)phenyl)-6-methoxy-2-methyl-1,2,3,4-tetrahydronaphthalen-1-ol (1.68 g, 3.19 mmol, 90%, 96.4% purity) as a white solid. LC-MS (ESI⁺) m/z: 508.3 (M+H)⁺.

Step 9: To a mixture of 2-cyclohexyl-1-(4-(4-(dimethoxymethyl)piperidin-1-yl)phenyl)-6-methoxy-2-methyl-1,2,3,4-tetrahydronaphthalen-1-ol (1.68 g, 1 Eq, 3.19 mmol), Pd/C (2 g, 10% Wt, 0.6 Eq, 2 mmol), H₂ (6.44 mg, 1 Eq, 3.19 mmol) in MeOH (25 mL) at r.t. and the mixture was degassed and purged with H₂ for 3 times. Then the mixture was stirred at 50° C. for 16 hour (15 psi). The mixture was filtered and concentrated to dryness in vacuo to give a white solid. The white solid was subjected by SFC: Column: DAICEL CHIRALPAK IG (250 mm*30 mm, 10 um); Mobile phase: A: Supercritical CO₂, B: MeOH (0.1% NH₃H₂O), A:B=40:40 at 300 mL/min. The pure fractions were collected respectively and the solvents were evaporated under vacuum to give the product 1-(4-((1S,2S)-2-cyclohexyl-6-methoxy-2-methyl-1,2,3,4-tetrahydronaphthalen-1-yl)phenyl)-4-(dimethoxymethyl)piperidine (400 mg, 787 μmol, 24.7%, 96.702% purity), 1-(4-((1R,2R)-2-cyclohexyl-6-methoxy-2-methyl-1,2,3,4-tetrahydronaphthalen-1-yl)phenyl)-4-(dimethoxymethyl)piperidine (330 mg, 671 μmol, 21.1%, 100% purity) 1-(4-((1S,2R)-2-cyclohexyl-6-methoxy-2-methyl-1,2,3,4-tetrahydronaphthalen-1-yl)phenyl)-4-(dimethoxymethyl)piperidine (80 mg, 0.16 mmol, 5.1%, 100% purity) and 1-(4-((1R,2S)-2-cyclohexyl-6-methoxy-2-methyl-1,2,3,4-tetrahydronaphthalen-1-yl)phenyl)-4-(dimethoxymethyl)piperidine (150 mg, 96.702% Wt, 1 Eq, 295 μmol) in DCM (2 mL) at 0° C. was added tribromoborane (222 mg, 885 μL, 1 M in DCM, 3 Eq, 885 μmol) with N₂. After addition, the mixture was stirred at 0° C. for 4 hour, and then the reaction was warmed to 25° C. and was stirred at this temperature for 12 hour. The reaction mixture was quenched with saturated NaHCO₃10 mL and stirred for 10 min at 0° C. Then the mixture was dissolved in water (30 mL) and extracted by ethyl acetate (40 mL). The combined organic layer was dried over Na₂SO₄, filtered and concentrated under vacuum to give the compound 1-(4-((1S,2S)-2-cyclohexyl-6-hydroxy-2-methyl-1,2,3,4-tetrahydronaphthalen-1-yl)phenyl)piperidine-4-carbaldehyde (125 mg, 290 μmol, 98.2%) as a yellow solid. LC-MS (ESI+) m/z: 432.2 (M+H)+.

Step 11: A mixture of 1-(4-(1S,2S)-2-cyclohexyl-6-hydroxy-2-methyl-1,2,3,4-tetrahydronaphthalen-1-yl)phenyl)piperidine-4-carbaldehyde (125 mg, 1 Eq, 290 μmol), (S)-3-(1-oxo-5-(piperazin-1-yl)isoindolin-2-yl)piperidine-2,6-dione benzenesulfonate (183 mg, 1.3 Eq, 376 μmol) and sodium acetate (119 mg, 5 Eq, 1.45 mmol) and acetic acid (52.2 mg, 50.0 μL, 3 Eq, 869 μmol) in DCM (2 mL) and MeOH (2 mL) at 25° C. for 1 hour, then sodium triacetoxyborohydride (123 mg, 2 Eq, 579 μmol) was added to the mixture and was stirred at 25° C. for 16 hour. The reaction was treated with H₂O (50 mL), extracted with EtOAc (50 mL). The combined extracts was dried over anhydrous Na₂SO₄, filtered and concentrated to dryness in vacuo to give a residue. The residue was purified by prep.HPLC (column: Welch Xtimate C18 150*30 mm*3 um mobile phase: [water (FA)-ACN]; B %: 20%-60%, 9 min). The aqueous phase was lyophilized to dryness to give (S)-3-(5-(4-((1-(4-((1S,2S)-2-cyclohexyl-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione (70.7 mg, 93.5 μmol, 32.3%, 98.415% purity) as a pink solid.

LC-MS (ESI+) m/z: 744.5 (M+H)+.

HPLC: 98.415%, purity at 220 nm.

b: retention time, 2.985 min; Area, 81.252%;

¹H NMR (400 MHz, DMSO-d₆) δ=10.96 (s, 1H), 9.13-8.84 (m, 1H), 8.17 (s, 0.390H), 7.52 (d, J=8.7 Hz, 1H), 7.10-7.04 (m, 2H), 6.84-6.78 (m, 2H), 6.77-6.71 (m, 2H), 6.62 (d, J=8.3 Hz, 1H), 6.51 (d, J=2.3 Hz, 1H), 6.39 (dd, J=2.4, 8.2 Hz, 1H), 5.06 (dd, J=5.1, 13.3 Hz, 1H), 4.39-4.28 (m, 1H), 4.26-4.16 (m, 1H), 3.73 (s, 1H), 3.65-3.57 (m, 2H), 3.29 (br s, 6H), 2.96-2.83 (m, 2H), 2.81-2.71 (m, 1H), 2.65-2.53 (m, 5H), 2.40-2.31 (m, 1H), 2.21 (br d, J=7.0 Hz, 2H), 2.13 (br d, J=11.1 Hz, 1H), 2.01-1.92 (m, 1H), 1.79 (br d, J=12.2 Hz, 2H), 1.74-1.63 (m, 3H), 1.62-1.51 (m, 4H), 1.26-1.15 (m, 2H), 1.06 (br dd, J=6.7, 12.0 Hz, 1H), 1.00-0.83 (m, 4H), 0.76 (s, 4H).

### Example 188. Preparation of (I-147) (S)-3-(5-(4-((1-(4-((1S,2S)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

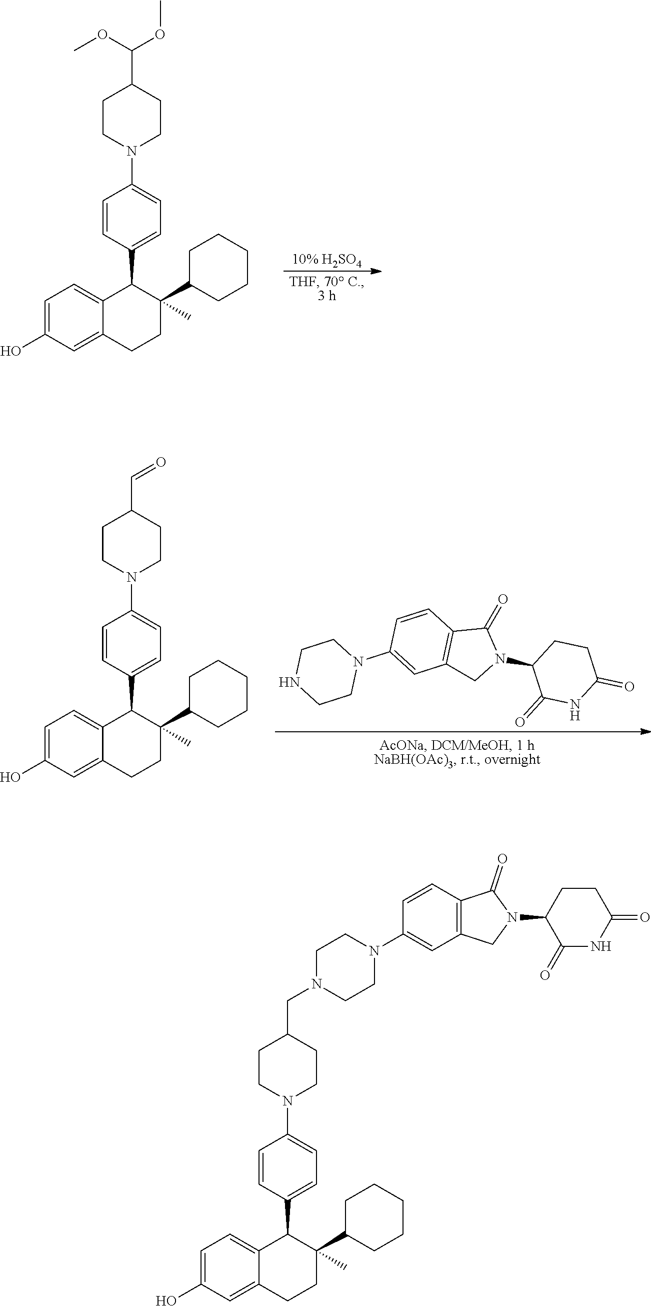

Step 1: To a solution of (5S,6S)-5-(4-(4-(dimethoxymethyl)piperidin-1-yl)phenyl)-6-methyl-6-phenyl-5,6,7,8-tetrahydronaphthalen-2-ol (85 mg, 1 Eq, 0.18 mmol) in THF (10 mL) was added sulfuric acid (0.18 g, 10 mL, 10% Wt, 1 Eq, 0.18 mmol). The mixture was stirred at 70° C. for 3 hour. LCMS showed 98.99% desired MS. The mixture was adjusted to pH=8-9 by progressively adding NaHCO₃. The reaction mixture was quenched by addition 100 mL EtOAc at 20° C., and then diluted with 50 mL H₂O and extracted with EtOAc 50 mL (25 mL*2). The combined organic layers were washed with 10 mL (10 mL*1), dried over Na₂SO₄. The reaction was filtered and concentrated under reduced pressure to give 1-(4-((1S,2S)-6-hydroxy-2-methyl-2-phenyl-1,2,3,4-tetrahydronaphthalen-1-yl)phenyl)piperidine-4-carbaldehyde (89 mg, 0.21 mmol, 98.986% purity) was obtained as a brown oil. LC-MS (ESI+) m/z: 444.3 (M+H)⁺.

Step 2: A mixture of 1-(4-((1S,2S)-6-hydroxy-2-methyl-2-phenyl-1,2,3,4-tetrahydronaphthalen-1-yl)phenyl)piperidine-4-carbaldehyde (89 mg, 98.986% Wt, 1 Eq, 0.21 mmol), (R)-3-(1-oxo-5-(piperazin-1-yl)isoindolin-2-yl)piperidine-2,6-dione, PhSO₃H salt (0.10 g, 1 Eq, 0.21 mmol) and sodium acetate (85 mg, 5 Eq, 1.0 mmol) in DCM (2 mL) and MeOH (2 mL) at 20° C. for 0.5 hour, pH=8, then acetic acid (37 mg, 36 μL, 3 Eq, 0.62 mmol) at 20° C. for 1 hour, pH=6, then added sodium triacetoxyhydroborate (88 mg, 2 Eq, 0.41 mmol) the mixture, and then the mixture was stirred at 25° C. for 16 hour. LCMS showed 86% desired MS. The reaction was concentrated to give (S)-3-(5-(4-((1-(4-((1R,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione as a brown oil. The mixture was filtered and the filter cake was washed by EtOAc (50 mL×3). The brown oil was purified by preparative high-performance liquid chromatography. Condition: Column: Welch Xtimate C18 150*30 mm*5 um, A: water (FA), B: CAN, at the beginning: A (82%) and B (18%), at the end: A: (52%) and B (48%), Gradient Time (min) 7; 100% B hold Time (min) 1.7, Flow Rate (ml/min) 25. The pure fractions were collected and the solvent was evaporated under vacuum. The aqueous layer was lyophilized to dryness to give (S)-3-(5-(4-((1-(4-((1S,2S)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (80.5 mg, 102 μmol, 49% yield, 93.16% purity) as a white solid.

LCMS: calc. for C₄₆H₅₁N₅O₄: 737.39, found: [M+H]⁺738.5.

HPLC: 93.16% purity at 220 nm.

¹H NMR (400 MHZ, DMSO-d₆) δ=10.95 (s, 1H), 9.08 (s, 1H), 8.13 (s, 0.262H), 7.51 (d, J=8.6 Hz, 1H), 7.23-7.12 (m, 4H), 7.09-7.02 (m, 3H), 6.67-6.59 (m, 2H), 6.48-6.38 (m, 3H), 6.29 (d. J=8.8 Hz, 2H), 5.04 (dd, J=5.1, 13.3 Hz, 1H), 4.32 (d, J=16.9 Hz, 1H), 4.23-4.16 (m, 1H), 4.02 (s, 1H), 3.51-3.40 (m, 4H), 3.28 (br s, 4H), 3.02 (br d, J=6.7 Hz, 1H), 2.97-2.85 (m, 2H), 2.62-2.53 (m, 3H), 2.45-2.32 (m, 4H), 2.22 (br s, 2H), 1.99-1.90 (m, 1H), 1.71 (br d, J=13.6 Hz, 4H), 1.31 (s, 3H), 1.17-1.04 (m, 2H)

SFC: retention time, 3.267 min; Area, 96.04%;

### Example 189. Preparation of (I-148) (S)-3-(5-(4-((1-(4-((1R,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

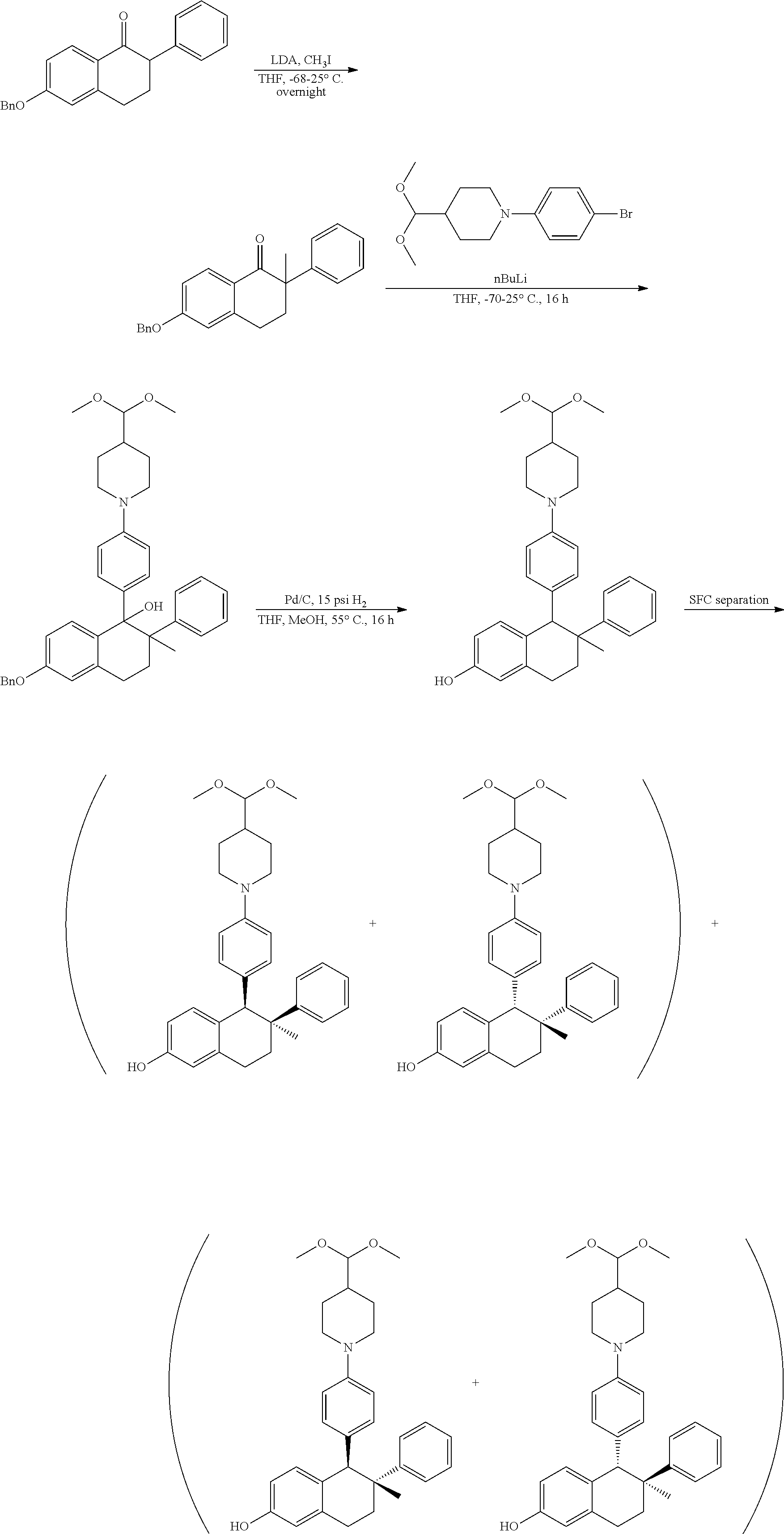

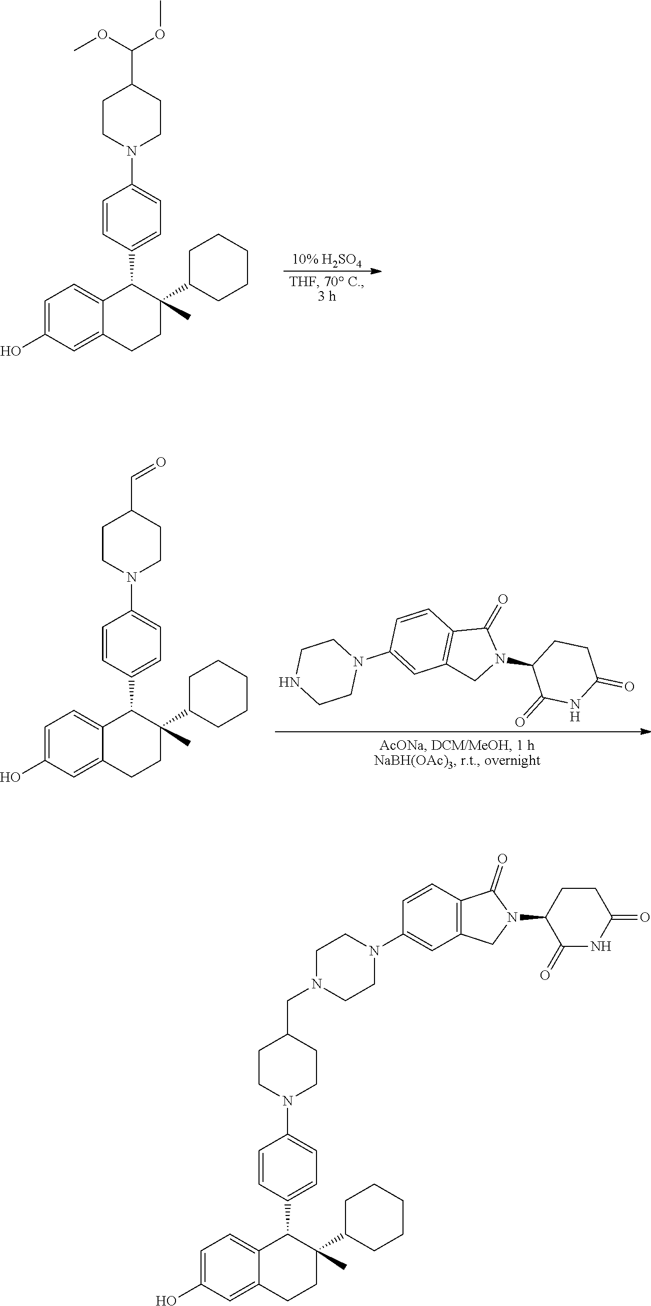

Step 1: To a solution of 6-(benzyloxy)-2-phenyl-3,4-dihydronaphthalen-1 (2H)-one (3 g, 1 Eq, 9 mmol) in THF (50 mL) was added LDA (1 g, 7 mL, 2 M, 1.5 Eq, 0.01 mol) at −68° C. The reaction mixture was stirred at −68° C. for 1 hour. A solution of Methyl iodide (4 g, 2 mL, 3 Eq, 0.03 mol)) was added to the mixture dropwise at −68° C. The reaction mixture was gradually warmed to 25° C. and stirred for 12 hour. LCMS showed 20.06% desired MS. TLC (petroleum ether:ethyl acetate=10:1, UV) showed one main new spot was observed. The reaction was quenched with water (150 mL) and extracted with ethyl acetate (150 mL*3). The organic layer was washed with brine (100 mL) and dried over Na₂SO₄ and concentrated in vacuo to give 6-(benzyloxy)-2-methyl-2-phenyl-3,4-dihydronaphthalen-1 (2H)-one as a brown oil. The brown oil was subjected to column chromatography over silica gel (gradient elution: 0-10% EtOAc). LCMS showed 81.5% desired MS. The desired fractions were collected, and concentrated to dryness in vacuo to give 6-(benzyloxy)-2-methyl-2-phenyl-3,4-dihydronaphthalen-1 (2H)-one (810 mg, 1.93 mmol, 20% yield, 81.5% purity) as a yellow oil. LC-MS (ESI⁺) m/z: 343.1 (M+H)⁺.

Step 2:1-(4-bromophenyl)-4-(dimethoxymethyl)piperidine (796 mg, 1.5 Eq, 2.53 mmol), a stir bar, and THF (30 mL) were added an oven-dried and nitrogen-purged three round-bottomed flask (100 mL), which was subsequently evacuated and refilled with argon (×3), before the reaction vessel was cooled to −70° C. (dry ice/EtOH). The resulting mixture treated with n-Butyllithium, 2.5M in hexane (152 mg, 946 μL, 2.5 M, 1.4 Eq, 2.37 mmol), dropwise over 2 min at −70° C. (dry ice/EtOH), while stirring an additional 30 min at −70° C. (dry ice/EtOH). The resulting mixture treated with 6-(benzyloxy)-2-methyl-2-phenyl-3,4-dihydronaphthalen-1 (2H)-one (710 mg, 81.5% Wt, 1 Eq, 1.69 mmol) in THF (5 mL), dropwise over 5 min, and the reaction vessel removed to the r.t., while stirring an additional 16 hour at 25° C. LCMS showed 28.61% desired MS. TLC (petroleum ether:ethyl acetate=10:1, UV) showed one main new spot was observed. The reaction was quenched with water (150 mL) and extracted with ethyl acetate (150 mL*3). The organic layer was washed with brine (50 mL) and dried over Na₂SO₄ and concentrated in vacuo to give 6-(benzyloxy)-1-(4-(4-(dimethoxymethyl)piperidin-1-yl)phenyl)-2-methyl-2-phenyl-1,2,3,4-tetrahydronaphthalen-1-ol as a brown oil. The brown oil was subjected to column chromatography over silica gel (gradient elution: 0-10% EtOAc). The desired fractions were collected, and concentrated to dryness in vacuo to give 6-(benzyloxy)-1-(4-(4-(dimethoxymethyl)piperidin-1-yl)phenyl)-2-methyl-2-phenyl-1,2,3,4-tetrahydronaphthalen-1-ol (600 mg, 1.04 mmol, 61.5% yield, 100% purity) as a yellow oil. LC-MS (ESI⁺) m/z: 578.3 (M+H)⁺.

Step 3: A mixture of 6-(benzyloxy)-1-(4-(4-(dimethoxymethyl)piperidin-1-yl)phenyl)-2-methyl-2-phenyl-1,2,3,4-tetrahydronaphthalen-1-ol (660 mg, 1 Eq, 1.14 mmol), Pd/C (700 mg, 10% Wt, 0.576 Eq, 658 μmol) in THF (8 mL) and MeOH (8 mL) was degassed and purged with H₂ for 3 times, and then the mixture was stirred at 55° C. for 16 hour under H₂ atmosphere (15 psi). LCMS showed 97.53% desired. The reaction was filtered and concentrated to dryness in vacuo to give 5-(4-(4-(dimethoxymethyl)piperidin-1-yl)phenyl)-6-methyl-6-phenyl-5,6,7,8-tetrahydronaphthalen-2-ol (500 mg, 1.03 mmol, 90.5% yield, 97.53% purity) as a brown oil. LC-MS (ESI+) m/z: 472.2 (M+H)⁺.

Step 4: The brown oil was purified by SFC. Condition: 0.1% NH₃H₂O ETOH. Column: DAICEL CHIRALCEL OJ (250 mm*30 mm, 10 um), at the beginning: A (60%) and B (40%), at the end: A: (60%) and B (40%), Flow Rate (ml/min) 80. The pure fractions were collected and the solvent was evaporated under vacuum. The aqueous layer was lyophilized to dryness to give (5R,6R)-5-(4-(4-(dimethoxymethyl)piperidin-1-yl)phenyl)-6-methyl-6-phenyl-5,6,7,8-tetrahydronaphthalen-2-ol (177 mg, 373 μmol, 99.4% purity) as a purple solid, (5S,6S)-5-(4-(4-(dimethoxymethyl)piperidin-1-yl)phenyl)-6-methyl-6-phenyl-5,6,7,8-tetrahydronaphthalen-2-ol (175 mg, 371 μmol, 100% purity) as a purple solid, (5R,6S)-5-(4-(4-(dimethoxymethyl)piperidin-1-yl)phenyl)-6-methyl-6-phenyl-5,6,7,8-tetrahydronaphthalen-2-ol (62 mg, 0.13 mmol, 100% purity) as a purple solid, (5S,6R)-5-(4-(4-(dimethoxymethyl)piperidin-1-yl)phenyl)-6-methyl-6-phenyl-5,6,7,8-tetrahydronaphthalen-2-ol (66 mg, 0.14 mmol, 100% purity) as a purple solid. LC-MS (ESI+) m/z: 472.1

Step 5: To a solution of (5R,6R)-5-(4-(4-(dimethoxymethyl)piperidin-1-yl)phenyl)-6-methyl-6-phenyl-5,6,7,8-tetrahydronaphthalen-2-ol (85 mg, 1 Eq, 0.18 mmol) in THF (10 mL) was added sulfuric acid (0.18 g, 10 mL, 10% Wt, 1 Eq, 0.18 mmol). The mixture was stirred at 70° C. for 3 hour. LCMS showed 97.47% desired MS. The mixture was adjusted to pH=8-9 by progressively adding NaHCO₃. The reaction mixture was quenched by addition 100 mL EtOAc at 20° C., and then diluted with H₂O (50 mL) and extracted with EtOAc 50 mL (25 mL*2). The combined organic layers were washed with 10 mL (10 mL*1), dried over Na₂SO₄. The reaction was filtered and concentrated under reduced pressure to give 1-(4-((1R,2R)-6-hydroxy-2-methyl-2-phenyl-1,2,3,4-tetrahydronaphthalen-1-yl)phenyl)piperidine-4-carbaldehyde (89 mg, 0.20 mmol, 97.47% purity) was obtained as a brown oil. LC-MS (ESI+) m/z: 444.3 (M+H)⁺.

Step 6: A mixture of 1-(4-((1R,2R)-6-hydroxy-2-methyl-2-phenyl-1,2,3,4-tetrahydronaphthalen-1-yl)phenyl)piperidine-4-carbaldehyde (89 mg, 1 Eq, 0.21 mmol), (S)-3-(1-oxo-5-(piperazin-1-yl)isoindolin-2-yl)piperidine-2,6-dione, PhSO₃H salt (0.10 g, 1 Eq, 0.21 mmol) and sodium acetate (86 mg, 5 Eq, 1.0 mmol) in DCM (2 mL) and MeOH (2 mL) at 20° C. for 0.5 hour, pH=8, then acetic acid (38 mg, 36 μL, 3 Eq, 0.63 mmol) at 20° C. for 1 hour, pH=6, then added sodium triacetoxyhydroborate (89 mg, 2 Eq. 0.42 mmol) the mixture, and then the mixture was stirred at 25° C. for 16 hour. LCMS showed 78% desired MS. The reaction was concentrated to give (S)-3-(5-(4-((1-(4-((1R,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione as a yellow oil. The mixture was filtered and the filter cake was washed by EtOAc (50 mL×3). The yellow oil was purified by preparative high-performance liquid chromatography. Condition: Column: Welch Xtimate C18 150*30 mm*5 um, A: water(FA), B:CAN, at the beginning: A (82%) and B (18%), at the end: A: (52%) and B (48%), Gradient Time (min) 7; 100% B hold Time (min) 2.5, Flow Rate (ml/min) 25. The pure fractions were collected and the solvent was evaporated under vacuum. The aqueous layer was lyophilized to dryness to give (S)-3-(5-(4-((1-(4-((1R,2R)-6-hydroxy-2-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (79.4 mg, 101 μmol, 48% yield, 93.93% purity) as a white solid.

LCMS: calc. for C46H15N5O4: 737.39, found: [M+H]+ 738.5.

HPLC: 93.93% purity at 220 nm.

¹H NMR (400 MHz, DMSO-d₆) δ=10.95 (s, 1H), 9.08 (s, 1H), 8.13 (s, 0.260H), 7.52 (d, J=8.5 Hz, 1H), 7.23-7.13 (m, 4H), 7.06 (s, 3H), 6.66-6.60 (m, 2H), 6.48-6.39 (m, 3H), 6.29 (d, J=8.7 Hz, 2H), 5.04 (dd, J=5.1, 13.2 Hz, 1H), 4.32 (d, J=17.0 Hz, 1H), 4.23-4.15 (m, 1H), 4.02 (s, 1H), 3.51-3.41 (m, 4H), 3.29-3.19 (m, 4H), 3.02 (br d, J=6.6 Hz, 1H), 2.97-2.85 (m, 2H), 2.61-2.54 (m, 3H), 2.48-2.33 (m, 5H), 2.26-2.19 (m, 1H), 1.99-1.91 (m, 1H), 1.76-1.57 (m, 4H), 1.31 (s, 3H), 1.11 (br d, J=9.4 Hz, 2H)

SFC: retention time, 3.074 min: Area, 96%;

### Example 190. Preparation of (I-85) (S)-3-(5-(4-((1-(4-((R)-6′-hydroxy-3′,4′-dihydro-1′H-spiro[cyclohexane-1,2′-naphthalen]-1′-yl)phenyl)piperidin-4-yl)methyl)piperazin-1-yl)-1-oxoisoindolin-2-yl)piperidine-2,6-dione

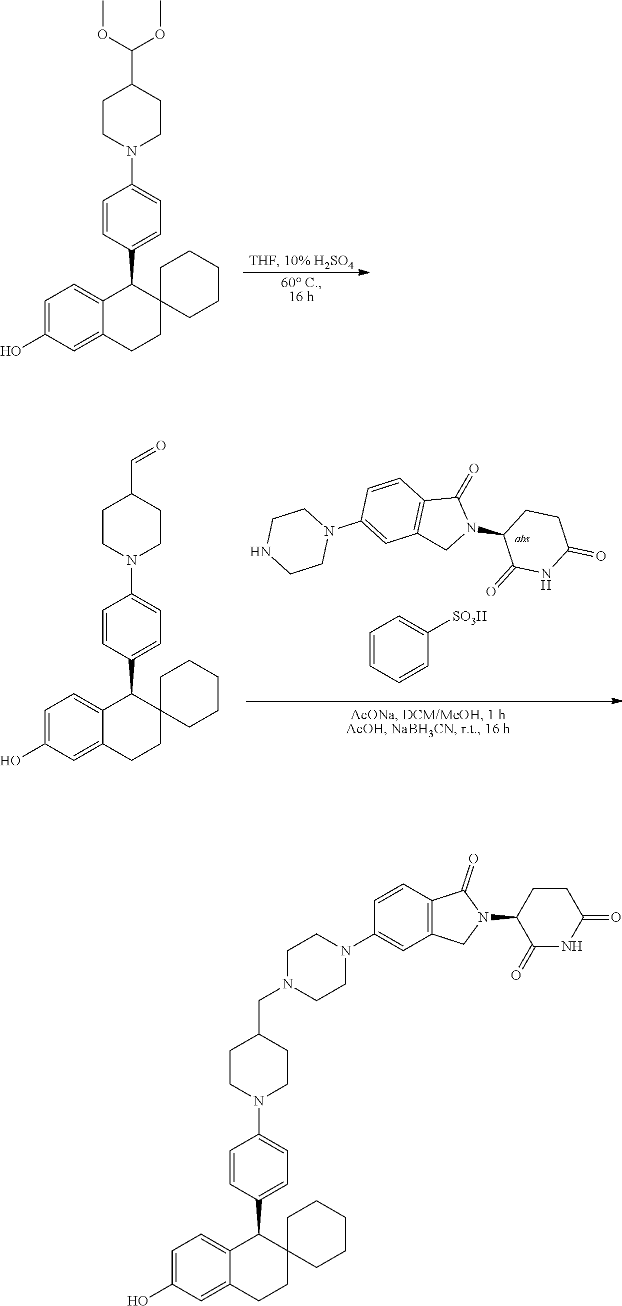

Step 1: A mixture of (R)-1′-(4-(4-(dimethoxymethyl)piperidin-1-yl)phenyl)-3′,4′-dihydro-1′H-spiro[cyclohexane-1,2′-naphthalen]-6′-ol (100 mg, 100% Wt, 1 Eq, 222 μmol) in 10% H₂SO₄ (1.5 mL) and THF (1.5 mL), then the mixture was stirred at 60° C. for 16 hour. The solvent was removed under vacuum. The aqueous solution was added NaHCO₃ to adjust pH=6. The residue was dissolved in water (20 mL) and extracted by ethyl acetate (20 mL). The combined organic layer was dried over Na₂SO₄, filtered and concentrated under vacuum to give (R)-1-(4-(6′-hydroxy-3′,4′-dihydro-1′H-spiro[cyclohexane-1,2′-naphthalen]-1′-yl)phenyl)piperidine-4-carbaldehyde (90 mg, 0.22 mmol, 100% yield) as a yellow oil. LC-MS (ESI+) m/z: 404.2 (M+H)+.

Step 2: A mixture of (R)-1-(4-(6′-hydroxy-3′,4′-dihydro-1′H-spiro[cyclohexane-1,2′-naphthalen]-1′-yl)phenyl)piperidine-4-carbaldehyde (90 mg, 1 Eq, 0.22 mmol), (S)-3-(1-oxo-5-(piperazin-1-yl)isoindolin-2-yl)piperidine-2,6-dione benzenesulfonate (0.14 g, 1.3 Eq, 0.29 mmol) and Sodium acetate (91 mg, 5 Eq, 1.1 mmol) and Acetic acid (40 mg, 38 μL, 3 Eq, 0.67 mmol) in DCM (2 mL) and MeOH (2 mL) at 25° C. for 1 hour, then Sodium triacetoxyborohydride (95 mg, 2 Eq. 0.45 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um mobile phase: [water(FA)-ACN]: B %: 21%-51%, 7 min). The aqueous phase was lyophilized to dryness to give (S)-3-(5-(4-((1-(4-((R)-6′-hydroxy-3′,4′-dihydro-1′H-spirolcyclohexane-1,2′-naphthalen]-1′-yl)phenyl)piperidin-4-yl)methyl)piperazin-1-yl)-1-oxoisoindolin-2-yl)piperidine-2,6-dione (53 mg, 73 μmol, 33% yield, 98.7% purity) as a white solid.

LC-MS (ESI+) m/z: 716.3 (M+H)+

HPLC: 98.709%, purity at 220 nm.

SFC: retention time, 3.011 min: Area, 81.517%;

¹H NMR (400 MHz, DMSO-d6) δ=10.99-10.93 (m, 1H), 9.07-8.94 (m, 1H), 8.17-8.12 (m, 0.428H), 7.58-7.48 (m, 1H), 7.11-7.02 (m, 2H), 6.82-6.71 (m, 4H), 6.60-6.54 (m, 1H), 6.53-6.49 (m, 1H), 6.44-6.38 (m, 1H), 5.09-5.01 (m, 1H), 4.37-4.29 (m, 1H), 4.25-4.17 (m, 1H), 3.64-3.54 (m, 4H), 3.32-3.25 (m, 7H), 2.96-2.84 (m, 1H), 2.81-2.66 (m, 2H), 2.64-2.56 (m, 3H), 2.40-2.31 (m, 1H), 2.27-2.18 (m, 2H), 2.02-1.90 (m, 1H), 1.85-1.75 (m, 2H), 1.72-1.54 (m, 3H), 1.52-1.41 (m, 4H), 1.40-1.28 (m, 2H), 1.26-1.06 (m, 5H), 0.84-0.73 (m, 1H).

### Example 191. Preparation of (I-142) (S)-3-(5-(4-((1-(4-((S)-6′-hydroxy-3′,4′-dihydro-1′H-spirolcyclohexane-1,2′-naphthalen]-1′-yl)phenyl)piperidin-4-yl)methyl)piperazin-1-yl)-1-oxoisoindolin-2-yl)piperidine-2,6-dione

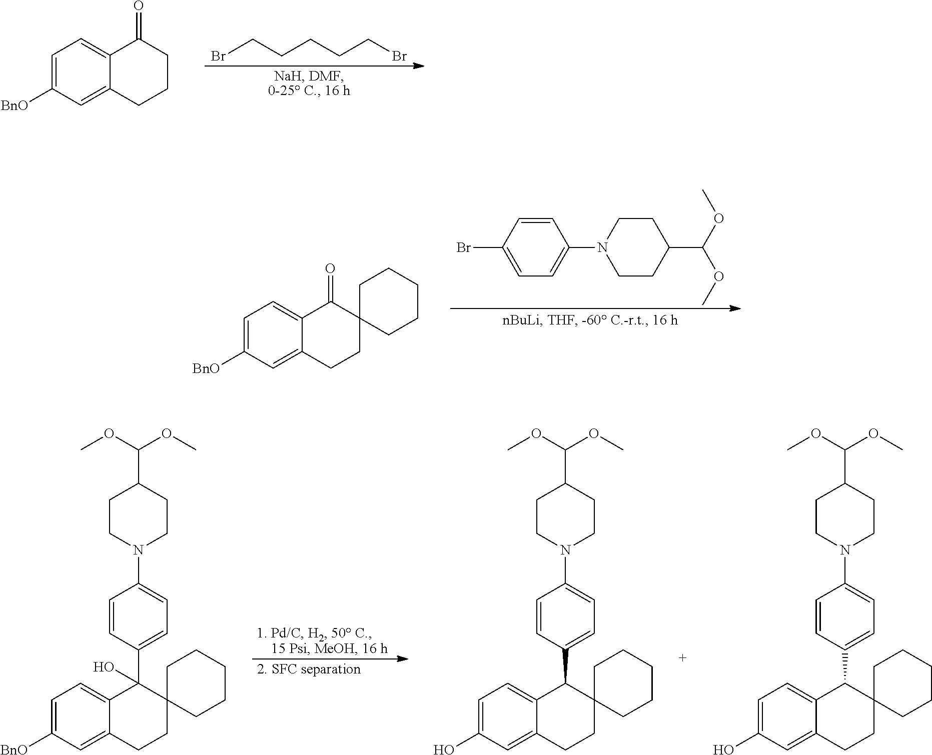

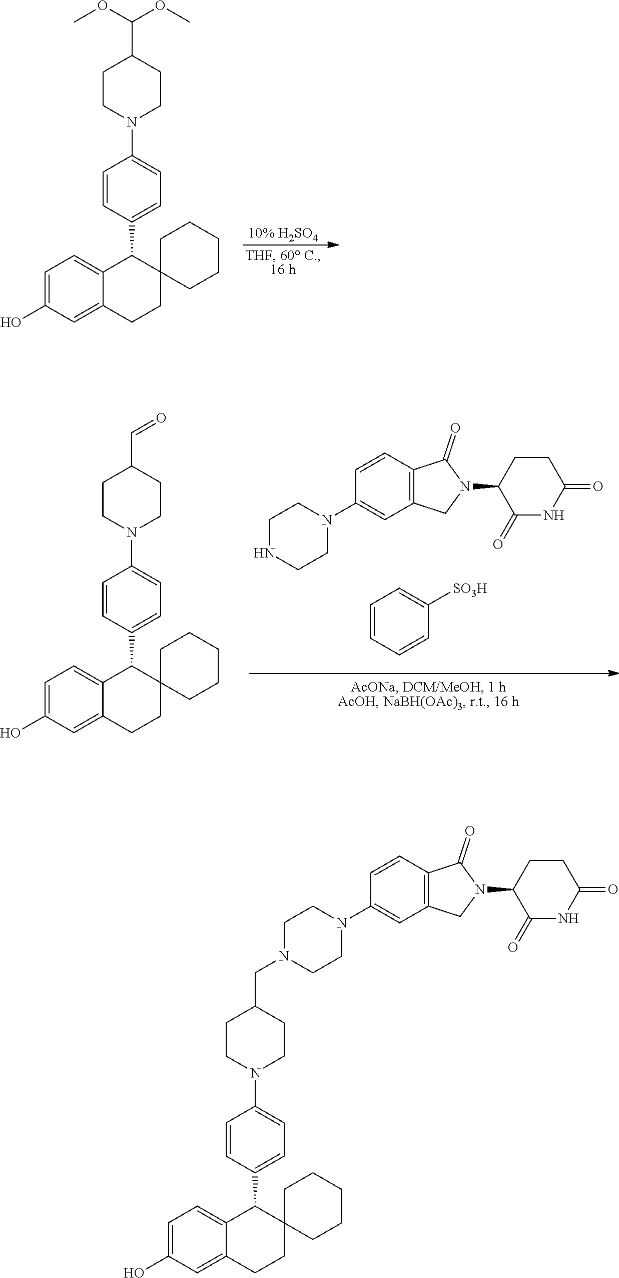

Step 1: To a cooled stirred (ice-salt) suspension of sodium hydride (1 g, 0.8 mL, 60% Wt, 2 Eq, 0.02 mol) in DMF (20 mL) was added dropwise a solution of 6-(benzyloxy)-3,4-dihydronaphthalen-1(2H)-one (3 g, 1 Eq. 0.01 mol), then stirred at 0° C. for 2 hour. After this time, 1,5-dibromo-pentane (4 g, 2 mL, 1.5 Eq, 0.02 mol) was added to the reaction and stirred at 25° C. for 16 hour. Then the mixture was cooled to mom temperature. The mixture was treated with H₂O (100 mL), extracted with EtOAc (150 mL). The combined extracts was dried over anhydrous Na₂SO₄, filtered and concentrated to dryness in vacuo to give a yellow oil. The yellow oil was purified by flash column (petroleum ether/ethyl acetate from 100/0 to 90/10) and the organic layer was concentrated in vacuo to give 6′-(benzyloxy)-3′,4′-dihydro-1′H-spiro[cyclohexane-1,2′-naphthalen]-1′-one (2.1 g, 5.9 mmol, 50%, 90% Purity) as a yellow oil. LC-MS (ESI+) m/z: 321.2 (M+H)+.

Step 2: To a mixture of 1-(4-bromophenyl)-4-(dimethoxymethyl)piperidine (3.7 g, 2 Eq, 12 mmol) in THF (20 mL) at r.t. and the mixture was degassed and purged with N₂ for 10 minutes. Then the mixture was stirred until it reached −60° C. n-Butyllithium, 2.5M in hexane (1.1 g, 7.1 mL, 2.5 molar, 3 Eq, 18 mmol) was added to the mixture and stirred at −78° C. for 1 hour, then 6′-(benzyloxy)-3′,4′-dihydro-1′H-spiro[cyclohexane-1,2′-naphthalen]-1′-one (2.1 g, 90% Wt, 1 Eq, 5.9 mmol) was added to the mixture at −78° C. and stirred at 25° C. for 16 hour. Then the reaction was cooled to room temperature. The mixture was treated with H₂O (100 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜50% ethyl acetate in petroleum ether). The pure fractions were collected and concentrated to dryness in vacuo to give 6′-(benzyloxy)-1′-(4-(4-(dimethoxymethyl)piperidin-1-yl)phenyl)-3′,4′-dihydro-1′H-spiro[cyclohexane-1,2′-naphthalen]-1′-ol (2.9 g, 5.2 mmol, 88%, 100% purity) as a yellow solid. LC-MS (ESI+) m/z: 556.2 (M+H)+.

Step 3: A mixture of 6′-(benzyloxy)-1′-(4-(4-(dimethoxymethyl)piperidin-1-yl)phenyl)-3′,4′-dihydro-1′H-spiro[cyclohexane-1,2′-naphthalen]-1′-ol (1 g, 1 Eq, 2 mmol), Pd/C (1 g, 10 Wt %, 0.5 Eq, 0.9 mmol), H₂ (4 mg, 1 Eq, 2 mmol) in MeOH (10 mL). The suspension was degassed and purged with H₂ for 3 times. The mixture was stirred under H₂ (15 psi) at 50° C. for 24 hour. Then the reaction was cooled to room temperature. The mixture was filtered and concentrated to dryness in vacuo to give a yellow oil. The white solid was subjected by SFC: Column: DAICEL CHIRALPAK IG (250 mm*30 mm, 10 um); Mobile phase: A: Supercritical CO₂, B: MeOH (0.1% NH₃H₂O), A:B=60:60 at 80 mL/min. The aqueous phase was lyophilized to dryness to give (R)-1′-(4-(4-(dimethoxymethyl)piperidin-1-yl)phenyl)-3′,4′-dihydro-1′H-spiro[cyclohexane-1,2′-naphthalen]-6′-ol (150 mg, 334 μmol, 20%, 100% Purity) and (S)-1′-(4-(4-(dimethoxymethyl)piperidin-1-yl)phenyl)-3′,4′-dihydro-1′H-spiro[cyclohexane-1,2′-naphthalen]-6′-ol (150 mg, 334 μmol, 20%, 100% Purity) as a yellow solid. LC-MS (ESI⁺) m/z: 450.2 (M+H)⁺.

Step 4: A mixture of (S)-1′-(4-(4-(dimethoxymethyl)piperidin-1-yl)phenyl)-3′,4′-dihydro-1′H-spiro[cyclohexane-1,2′-naphthalen]-6′-ol (100 mg, 100% Wt, 1 Eq, 222 μmol) in 10% H₂SO₄ (1.5 mL) and THF (1.5 mL), then the mixture was stirred at 60° C. for 16 hour. The solvent was removed under vacuum. The aqueous solution was added NaHCO₃ to adjust pH=7. The residue was dissolved in water (20 mL) and extracted by ethyl acetate (20 mL). The combined organic layer was dried over Na₂SO₄, filtered and concentrated under vacuum to give (S)-1-(4-(6′-hydroxy-3′,4′-dihydro-1′H-spiro[cyclohexane-1,2′-naphthalen]-1′-yl)phenyl)piperidine-4-carbaldehyde (90 mg, 0.22 mmol, 100% yield) as a yellow oil. LC-MS (ESI+) m/z: 404.2 (M+H)+.

Step 5: A mixture of (S)-1-(4-(6′-hydroxy-3′,4′-dihydro-1′H-spiro[cyclohexane-1,2′-naphthalen]-1′-yl)phenyl)piperidine-4-carbaldehyde (90 mg, 1 Eq, 0.22 mmol), (S)-3-(1-oxo-5-(piperazin-1-yl)isoindolin-2-yl)piperidine-2,6-dione benzenesulfonate (0.14 g, 1.3 Eq, 0.29 mmol) and Sodium acetate (91 mg, 5 Eq, 1.1 mmol) and Acetic acid (40 mg, 38 μL, 3 Eq, 0.67 mmol) in DCM (2 mL) and MeOH (2 mL) at 25° C. for 1 hour, then sodium triacetoxyborohydride (95 mg, 2 Eq, 0.45 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um mobile phase: [water(FA)-ACN]; B %: 21%-51%, 7 min). The aqueous phase was lyophilized to dryness to give (S)-3-(5-(4-((1-(4-((S)-6′-hydroxy-3′,4′-dihydro-1′H-spiro[cyclohexane-1,2′-naphthalen]-1′-yl)phenyl)piperidin-4-yl)methyl)piperazin-1-yl)-1-oxoisoindolin-2-yl)piperidine-2,6-dione (81.1 mg, 112 μmol, 50% yield, 98.783% purity) as a white solid.

LC-MS (ESI+) m/z: 716.3 (M+H)⁺

HPLC: 98.783%, purity at 220 nm.

SFC: retention time, 2.998 min; Area, 82.319%;

¹H NMR (400 MHZ, DMSO-d₆) δ=11.00-10.93 (m, 1H), 9.06-8.95 (m, 1H), 8.17-8.12 (m, 0.392H), 7.57-7.50 (m, 1H), 7.10-7.02 (m, 2H), 6.83-6.71 (m, 4H), 6.61-6.54 (m, 1H), 6.53-6.48 (m, 1H), 6.45-6.37 (m, 1H), 5.10-5.01 (m, 1H), 4.37-4.30 (m, 1H), 4.25-4.17 (m, 1H), 3.62-3.54 (m, 4H), 3.30 (br s, 7H), 2.96-2.85 (m, 1H), 2.80-2.65 (m, 2H), 2.63-2.56 (m, 3H), 2.42-2.32 (m, 1H), 2.29-2.22 (m, 2H), 2.01-1.92 (m, 1H), 1.85-1.75 (m, 2H), 1.73-1.54 (m, 3H), 1.50-1.41 (m, 4H), 1.40-1.28 (m, 2H), 1.26-1.08 (m, 5H), 0.84-0.73 (m, 1H)

### Example 192. Preparation of (I-151) (S)-3-(5-(4-((1-(4-((1R,2S)-2-cyclohexyl-6-hydroxy-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione and (I-152) (S)-3-(5-(4-((1-(4-((1S,2R)-2-cyclohexyl-6-hydroxy-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione

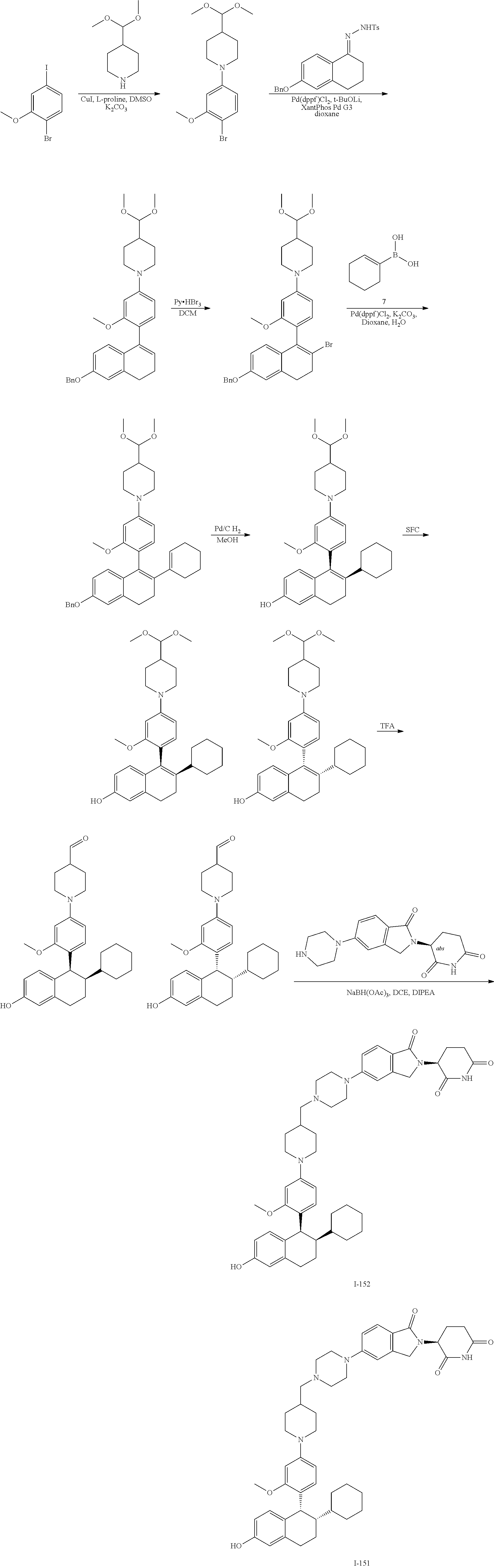

Step 1: A mixture of 1-bromo-4-iodo-2-methoxybenzene (1.5 g, 4.79 mmol, 1.0 eq), 4-(dimethoxymethyl)piperidine (915.88 mg, 5.75 mmol, 1.2 eq), CuI (456.45 mg, 2.40 mmol, 0.5 eq), L-proline (275.93 mg, 2.40 mmol, 0.5 eq) and K₂CO₃ (1.32 g, 9.59 mmol, 2.0 eq) in DMSO (15 mL) was degassed and purged with N₂ for 3 times, and then the mixture was stirred at 90° C. for 2 hr under N₂ atmosphere. The mixture was concentrated in reduced pressure. The residue was poured into water (15 mL). The aqueous phase was extracted with ethyl acetate (30 mL×3). The combined organic phase was washed with brine (15 mL), dried with Na₂SO₄, filtered and concentrated in vacuum. The residue was purified by silica gel chromatography (Petroleum ether/Ethyl acetate=3/1). LCMS indicated no desired compound. 1-(4-bromo-3-methoxyphenyl)-4-(dimethoxymethyl)piperidine (1 g, 2.90 mmol, 60.60% yield) was obtained as oil. LC-MS (ESI+) m/z: 345.7 (M+H⁺).

Step 2: A mixture of 1-(4-bromo-3-methoxyphenyl)-4-(dimethoxymethyl)piperidine (1.0 g, 2.90 mmol, 1.0 eq), (E)-N′-(6-(benzyloxy)-3,4-dihydronaphthalen-1 (2H)-ylidene)-4-methylbenzenesulfonohydrazide (1.34 g, 3.20 mmol, 1.1 eq), Pd(dppf)Cl₂ (212.55 mg, 290.49 μmol, 0.1 eq), t-BuOLi (697.66 mg, 8.71 mmol, 785.65 μL, 3 eq) and XantPhos-Pd-G3 (245.89 mg, 290.49 μmol, 0.1 eq) in dioxane (15 mL) was degassed and purged with N₂ for 3 times, and then the mixture was stirred at 100° C. for 16 hr under N₂ atmosphere. LCMS showed that the starting material was consumed and the desired product was detected. The residue was poured into water (15 mL). The aqueous phase was extracted with ethyl acetate (30 mL×3). The combined organic phase was washed with brine (15 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3/1) to afford 5 (1.02 g, 2.04 mmol, 70.28% yield) as yellow oil. The reaction mixture was filtered under reduced pressure to give a compound. 1-(4-(6-(benzyloxy)-3,4-dihydronaphthalen-1-yl)-3-methoxyphenyl)-4-(dimethoxymethyl)piperidine (1.02 g, 2.04 mmol, 70.28% yield) was obtained as oil. LC-MS (ESI+) m/z: 500.3 (M+H⁺).

Step 3: To a solution of 1-(4-(6-(benzyloxy)-3,4-dihydronaphthalen-1-yl)-3-methoxyphenyl)-4-(dimethoxymethyl)piperidine (1.25 g, 2.50 mmol, 1.0 eq) in DCM (15 mL) was added Py·HBr₃ (800.13 mg, 2.50 mmol, 1.0 eq). The mixture was stirred at 25° C. for 1 hr. LCMS showed that the starting material was consumed and the desired product was detected. The residue was poured into water (15 mL). The aqueous phase was extracted with ethyl acetate (30 mL*3). The combined organic phase was washed with brine (15 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 6 (1 g, 1.73 mmol, 69.09% yield) as yellow oil. The reaction mixture was filtered under reduced pressure to give a compound. 1-(4-(6-(benzyloxy)-2-bromo-3,4-dihydronaphthalen-1-yl)-3-methoxyphenyl)-4-(dimethoxymethyl)piperidine (1 g, 1.73 mmol, 69.09% yield) was obtained as oil.

Step 4: A mixture of 1-(4-(6-(benzyloxy)-2-bromo-3,4-dihydronaphthalen-1-yl)-3-methoxyphenyl)-4-(dimethoxymethyl)piperidine (800 mg, 1.38 mmol, 1.0 eq), cyclohex-1-en-1-ylboronic acid (174.18 mg, 1.38 mmol, 1 eq), Pd(dppf)Cl₂ (101.18 mg, 138.28 μmol, 0.1 eq), K₂CO₃ (382.22 mg, 2.77 mmol, 2.0 eq) in dioxane (10 mL) and H₂O (2.5 mL) was degassed and purged with N₂ for 3 times, and then the mixture was stirred at 80° C. for 16 hr under N₂ atmosphere. LCMS showed that the starting material was consumed and the desired product was detected. The reaction mixture was filtered under reduced pressure to give a residue. The residue was poured into H₂O (15 mL). The aqueous phase was extracted with ethyl acetate (30 mL*3). The combined organic phase was washed with brine (15 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1:1) to afford 1-(4-(6-(benzyloxy)-2-(cyclohex-1-en-1-yl)-3,4-dihydronaphthalen-1-yl)-3-methoxyphenyl)-4-(dimethoxymethyl)piperidine (756 mg, 1.30 mmol, 94.30% yield) as yellow oil. LC-MS (ESI+) m/z: 580.6 (M+H)+.

Step 5: To a solution of 1-(4-(6-(benzyloxy)-2-(cyclohex-1-en-1-yl)-3,4-dihydronaphthalen-1-yl)-3-methoxyphenyl)-4-(dimethoxymethyl)piperidine (730 mg, 1.26 mmol, 1.0 eq) in MeOH (4 mL) was added Pd/C (803.98 mg, 755.48 μmol, 10% purity, 0.6 eq). The mixture was stirred under H₂ at 50° C. and 50 Psi for 16 hr. LCMS showed that the starting material was consumed and the desired product was detected. The reaction mixture was filtered under reduced pressure to give (5S,6R)-6-cyclohexyl-5-(4-(4-(dimethoxymethyl)piperidin-1-yl)-2-methoxyphenyl)-5,6,7,8-tetrahydronaphthalen-2-ol. (5R,6S)-6-cyclohexyl-5-(4-(4-(dimethoxymethyl)piperidin-1-yl)-2-methoxyphenyl)-5,6,7,8-tetrahydronaphthalen-2-ol (360 mg, 729.22 μmol, 57.91% yield) was obtained as oil. LC-MS (ESI+) m/z: 494.4 (M+H⁺).

Step 6: (5R,6S)-6-cyclohexyl-5-(4-(4-(dimethoxymethyl)piperidin-1-yl)-2-methoxyphenyl)-5,6,7,8-tetrahydronaphthalen-2-ol and (5S,6R)-6-cyclohexyl-5-(4-(4-(dimethoxymethyl)piperidin-1-yl)-2-methoxyphenyl)-5,6,7,8-tetrahydronaphthalen-2-ol (360 mg) was separated by chiral SFC(Column DAICEL CHIRALPAK AS (250 mm*30 mm, 10 um) Condition CO₂-EtOH (0.1% NH₃H₂O) Begin B 30% End B 30%) FlowRate (ml/min): 70) (5R,6S)-6-cyclohexyl-5-(4-(4-(dimethoxymethyl)piperidin-1-yl)-2-methoxyphenyl)-5,6,7,8-tetrahydronaphthalen-2-ol (110 mg), (5S,6R)-6-cyclohexyl-5-(4-(4-(dimethoxymethyl)piperidin-1-yl)-2-methoxyphenyl)-5,6,7,8-tetrahydronaphthalen-2-ol (128.6 mg).

Step 7: 1-(4-((1R,2S)-2-cyclohexyl-6-hydroxy-1,2,3,4-tetrahydronaphthalen-1-yl)-3-methoxyphenyl)piperidine-4-carbaldehyde: To a solution of (5S,6R)-6-cyclohexyl-5-(4-(4-(dimethoxymethyl)piperidin-1-yl)-2-methoxyphenyl)-5,6,7,8-tetrahydronaphthalen-2-ol (110 mg, 222.82 μmol, 1.0 eq) in DCM (10 mL) was added TFA (25.41 mg, 222.82 μmol, 16.55 μL, 1.0 eq). The mixture was stirred at 25° C. for 2 hr. LCMS showed that the starting material was consumed and the desired product was detected. The reaction mixture was filtered under reduced pressure to give a residue. 1-(4-((1R,2S)-2-cyclohexyl-6-hydroxy-1,2,3,4-tetrahydronaphthalen-1-yl)-3-methoxyphenyl)piperidine-4-carbaldehyde (80 mg, 178.73 μmol, 80.21% yield) was obtained as a solid. LC-MS (ESI+) m/z: 448.4 (M+H⁺).

1-(4-((1S,2R)-2-cyclohexyl-6-hydroxy-1,2,3,4-tetrahydronaphthalen-1-yl)-3-methoxyphenyl)piperidine-4-carbaldehyde: To a solution of (5R,6S)-6-cyclohexyl-5-(4-(4-(dimethoxymethyl)piperidin-1-yl)-2-methoxyphenyl)-5,6,7,8-tetrahydronaphthalen-2-ol (128.6 mg, 260.49 μmol, 1.0 eq) in DCM (12 mL) was added TFA (148.51 mg, 1.30 mmol, 96.75 μL, 5.0 eq). The mixture was stirred at 25° C. for 2 hr. LCMS showed that the starting material was consumed and the desired product was detected. The reaction mixture was filtered under reduced pressure to give a residue. 1-(4-((1S,2R)-2-cyclohexyl-6-hydroxy-1,2,3,4-tetrahydronaphthalen-1-yl)-3-methoxyphenyl)piperidine-4-carbaldehyde (85 mg, 189.90 μmol, 72.90% yield) was obtained as a solid. LC-MS (ESI+) m/z: 466.4 (M+H⁺).

Step 8: To a solution of 1-(4-(1R,2S)-2-cyclohexyl-6-hydroxy-1,2,3,4-tetrahydronaphthalen-1-yl)-3-methoxyphenyl)piperidine-4-carbaldehyde (60 mg, 134.05 μmol, 1.0 eq) in DCM (1 mL) and MeOH (1 mL) was added (S)-3-(1-oxo-5-(piperazin-1-yl)isoindolin-2-yl)piperidine-2,6-dione (44.02 mg, 134.05 μmol, 1.0 eq), C₆H₁₀BO₆Na (56.82 mg, 268.09 μmol, 2.0 eq) and NMM (1.36 mg, 13.40 μmol, 1.47 μL, 0.1 eq). The mixture was stirred at 25° C. for 16 hr. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water (FA)-ACN]; B %: 10%-40%, 12 min) to give (S)-3-(5-(4-((1-(4-((1R,2S)-2-cyclohexyl-6-hydroxy-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione (32.8 mg, 43.16 μmol, 32.20% yield) as a white solid. LC-MS (ESI⁺) m/z: 760.6 (M+H)⁺.

LCMS: calc. for C₄₆H₅₅N₅O₄:759.99, found: [M+H]⁺ 760.6.

HPLC: 100% purity at 220 nm.

¹H NMR (400 MHZ, METHANOL-d₄) δ=8.38 (s, 1H), 7.64 (d, J=8.5 Hz, 1H), 7.12-7.07 (m, 2H), 6.59-6.56 (m, 2H), 6.53-6.48 (m, 2H), 6.41 (dt, J=2.3, 8.6 Hz, 2H), 5.09 (dd, J=5.1, 13.4 Hz, 1H), 4.45-4.35 (m, 2H), 3.86 (s, 3H), 3.64 (brt, J=10.5 Hz, 2H), 3.41 (br s, 4H), 2.94-2.85 (m, 2H), 2.79-2.65 (m, 8H), 2.50-2.40 (m, 3H), 2.17-2.03 (m, 2H), 1.90 (br d, J=13.0 Hz, 2H), 1.83-1.49 (m, 8H), 1.44-1.34 (m, 2H), 1.25-0.97 (m, 6H), 0.79-0.67 (m, 1H).

Step 9: To a solution of 1-(4-((1S,2R)-2-cyclohexyl-6-hydroxy-1,2,3,4-tetrahydronaphthalen-1-yl)-3-methoxyphenyl)piperidine-4-carbaldehyde (75 mg, 167.56 μmol, 1.0 eq) in DCM (2 mL) and MeOH (2 mL) was added (S)-3-(1-oxo-5-(piperazin-1-yl)isoindolin-2-yl)piperidine-2,6-dione (55.02 mg, 167.56 μmol, 1.0 eq), C₆H₁₀BO₆Na (71.02 mg, 335.12 μmol, 2.0 eq) and NMM (1.36 mg, 13.40 μmol, 1.47 μL, 0.1 eq). The mixture was stirred at 25° C. for 16 hr. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water (FA)-ACN]; B %: 10%-40%, 12 min) to give (S)-3-(5-(4-((1-(4-((1S,2R)-2-cyclohexyl-6-hydroxy-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione (30.5 mg, 40.13 μmol, 23.95% yield) was obtained as a white solid. LC-MS (ESI⁺) m/z: 76.6 (M+H)⁺.

LCMS: calc. for C₄₆H₅₅N₅O₄:759.99, found: [M+H]⁺ 760.6.

HPLC: 100% purity at 220 nm.

¹H NMR (400 MHz, METHANOL-d₄) δ=8.39 (br s, 1H), 7.64 (d, J=8.5 Hz, 1H), 7.13-7.07 (m, 2H), 6.60-6.56 (m, 2H), 6.52-6.47 (m, 2H), 6.40 (dt, J=2.4, 8.9 Hz, 2H), 5.09 (dd, J=5.1, 13.2 Hz, 1H), 4.46-4.35 (m, 2H), 3.86 (s, 3H), 3.68-3.60 (m, 2H), 3.40 (br d, J=4.8 Hz, 4H), 2.94-2.85 (m, 2H), 2.79-2.65 (m, 8H), 2.51-2.40 (m, 3H), 2.16-2.03 (m, 2H), 1.90 (br d, J=12.8 Hz, 2H), 1.84-1.46 (m, 8H), 1.43-1.33 (m, 2H), 1.26-0.97 (m, 6H), 0.79-0.68 (m, 1H).

### Example 193. Preparation of (I-144) (S)-3-(5-(4-((1-(4-((1S,2S)-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione

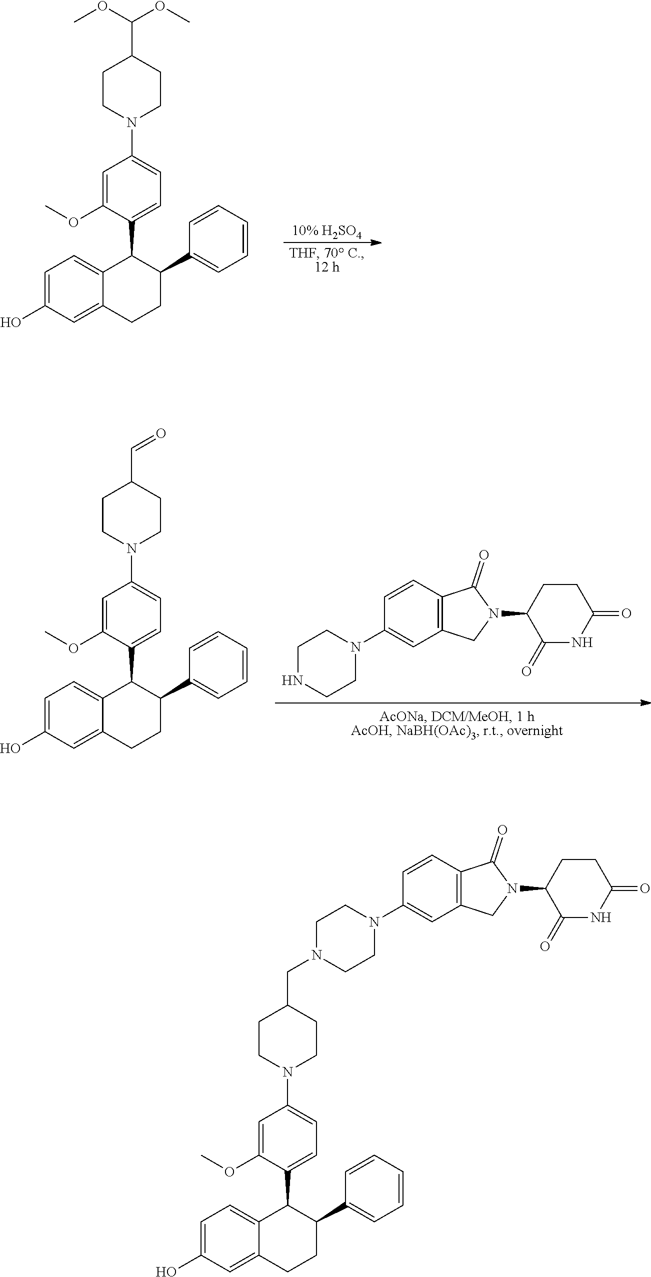

Step 1: To a solution of (5S,6S)-5-(4-(4-(dimethoxymethyl)piperidin-1-yl)-2-methoxyphenyl)-6-phenyl-5,6,7,8-tetrahydronaphthalen-2-ol (0.17 g, 5 mL, 1 Eq, 0.35 mmol) in THF (5 mL) was added sulfuric acid (34 mg, 1 Eq, 0.35 mmol). The mixture was stirred at 70° C. for 16 hour. LCMS showed 100% desired MS. The mixture was adjusted to pH 8-9 by progressively adding NaHCO₃. The reaction mixture was quenched by addition 100 mL EtOAc at 20° C., and then diluted with 50 mL H2O and extracted with EtOAc 50 mL (25 mL*2). The combined organic layers were washed with 10 mL (10 mL*1), dried over Na₂SO₄. The reaction was filtered and concentrated under reduced pressure to give 1-(4-((1S,2S)-6-hydroxy-2-phenyl-1,2,3,4-tetrahydronaphthalen-1-yl)-3-methoxyphenyl)piperidine-4-carbaldehyde (35 mg, 79 μmol, 23%, 100% purity) was obtained as a brown oil. LC-MS (ESI+) m/z: 460.3 (M+H)⁺.

Step 2: A mixture of 1-(4-((1S,2S)-6-hydroxy-2-phenyl-1,2,3,4-tetrahydronaphthalen-1-yl)-3-methoxyphenyl)piperidine-4-carbaldehyde (35 mg, 1 Eq, 79 μmol), (S)-3-(1-oxo-5-(piperazin-1-yl)isoindolin-2-yl)piperidine-2,6-dione, PhSO3H salt (40 mg, 1 Eq, 79 μmol) and sodium acetate (33 mg, 5 Eq, 0.40 mmol) in DCM (1 mL) and MeOH (1 mL) at 20° C. for 0.5 hour, pH=8, then acetic acid (14 mg, 14 μL, 3 Eq, 0.24 mmol) at 20° C. for 1 hour, pH=6, then added sodium triacetoxyhydroborate (34 mg, 2 Eq, 0.16 mmol) the mixture, and then the mixture was stirred at 25° C. for 16 hour. LCMS showed 89.04% desired MS. The reaction was concentrated to give (S)-3-(5-(4-((1-(4-((1S,2S)-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione as a brown oil. The mixture was filtered and the filter cake was washed by EtOAc (50 mL×3). The brown oil was purified by preparative high-performance liquid chromatography. Condition: Column: Xtimate C18 100*30 mm*3 um, A: water(FA), B: CAN, at the beginning: A (85%) and B (15%), at the end: A: (45%) and B (55%), Gradient Time (min) δ; 100% B hold Time (min) 2.5, Flow Rate (ml/min) 30. The pure fractions were collected and the solvent was evaporated under vacuum. The aqueous layer was lyophilized to dryness to give (S)-3-(5-(4-((1-(4-((1S,2S)-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione (17.5 mg, 23.1 μmol, 29%, 99.58% Purity) as a white solid.

LCMS: calc. for C₄₆H₅₁N₅O₅: 753.39, found: [M+H]⁺ 754.4.

HPLC: 99.6% purity at 220 nm.

¹H NMR (400 MHZ, DMSO-d₆) δ=ppm 10.95 (s, 1H) 9.07 (s, 1H) 8.15 (s, 0.092 H) 7.52 (d, J=8.70 Hz, 1H) 7.04-7.10 (m, 5H) 6.71-6.76 (m, 2H) 6.56 (d, J=8.58 Hz, 2H) 6.46 (dd, J=8.34, 2.27 Hz, 1H) 6.34-6.39 (m, 1H) 6.28-6.33 (m, 1H) 6.12 (d, J=1.67 Hz, 1H) 5.05 (dd, J=13.35, 5.01 Hz, 1H) 4.65 (d, J=5.01 Hz, 1H) 4.31-4.37 (m, 1H) 4.18-4.24 (m, 1H) 3.56 (br d, J=5.84 Hz, 2H) 3.29 (br s, 6H) 3.22 (br d, J=15.38 Hz, 2H) 2.95 (s, 4H) 2.86-2.92 (m, 2H) 2.55-2.63 (m, 3H) 2.53 (br s, 2H) 2.21 (br d, J=7.15 Hz, 3H) 1.94-2.01 (m, 1H) 1.77 (br d, J=13.11 Hz, 2H) 1.56-1.70 (m, 2H) 1.19 (br d, J=11.44 Hz, 2H).

SFC: retention time, 2.628 min; Area, 92.65%;

### Example 194. Preparation of (I-143) (S)-3-(5-(4-((1-(4-((1R,2R)-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione

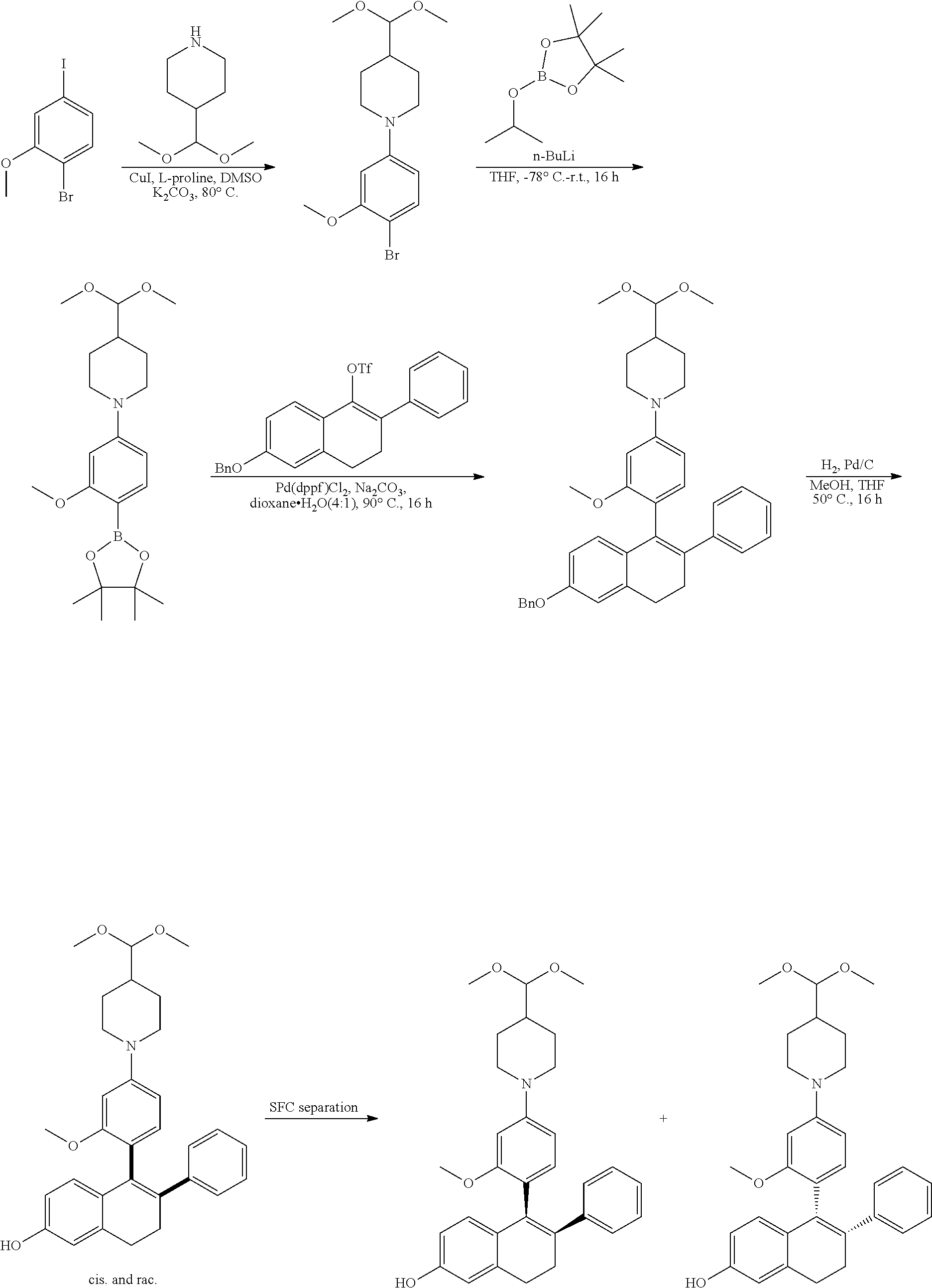

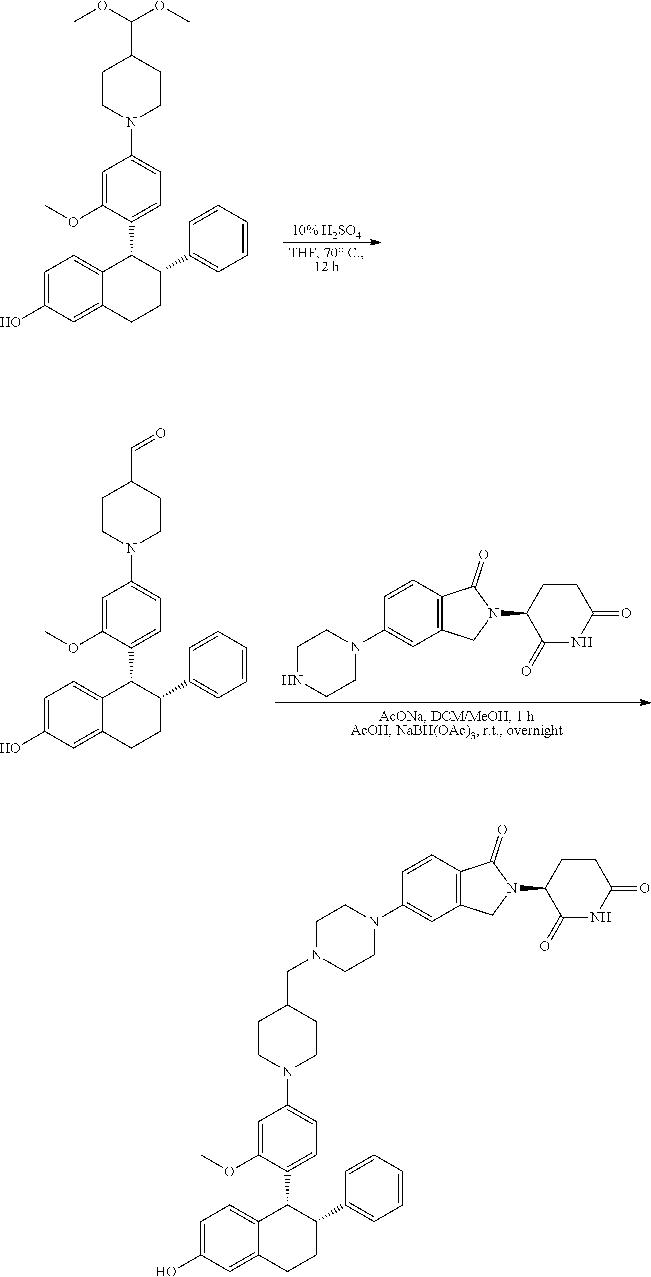

Step 1: To a solution of 1-bromo-4-iodo-2-methoxybenzene (3 g, 1 Eq, 0.01 mol) and 4-(dimethoxymethyl)piperidine (2 g, 2 mL, 1 Eq, 0.01 mol) in DMSO (150 mL) was added Cuprous iodide (0.4 g, 0.06 mL, 0.2 Eq, 2 mmol), L-Proline (0.4 g, 0.3 mL, 0.4 Eq, 4 mmol) and potassium carbonate (3 g, 1 mL, 2 Eq, 0.02 mol). The mixture was stirred at 80° C. for 12 hour. LCMS 75.8% desired MS was detected. TLC (petroleum ether:ethyl acetate=10:1, UV, R_{f}=0.3) showed one main new spot was observed. The reaction was quenched with water (50 mL) and extracted with ethyl acetate (50 mL*2). The organic layer was washed with brine (50 mL) and dried over Na₂SO₄ and concentrated in vacuo to give 1-(4-bromo-3-methoxyphenyl)-4-(dimethoxymethyl)piperidine as a brown oil. The brown oil was subjected to column chromatography over silica gel (gradient elution: 0-15% EtOAc). LCMS 100% desired MS was detected. The desired fractions were collected, and concentrated to dryness in vacuo to give 1-(4-bromo-3-methoxyphenyl)-4-(dimethoxymethyl)piperidine (1.6 g, 4.2 mmol, 40%, 90% purity) as a white solid. LC-MS (ESI+) m/z: 344.1 (M+H)⁺.

Step 2: To a solution of 1-(4-bromo-3-methoxyphenyl)-4-(dimethoxymethyl)piperidine (1.6 g, 1 Eq, 4.6 mmol) in THF (100 mL) was added n-Butyllithium, 2.5 M in hexane (0.33 g, 2.0 mL, 2.5 molar, 1.1 Eq, 5.1 mmol) at −68° C. over 5 min. After addition, the mixture was stirred at this temperature for 1.5 hour, and then isopropoxyboronic acid pinacol ester (1.1 g, 1.2 mL, 1.3 Eq, 6.0 mmol) was added dropwise at −68° C. The resulting mixture was stirred at 25° C. for 16 hour, LCMS showed 14.27% desired MS. TLC (petroleum ether:ethyl acetate=5:1, UV) showed one main new spot was observed. The reaction was quenched with water (100 mL) and extracted with ethyl acetate (200 mL*2). The organic layer was washed with brine (50 mL) and dried over Na₂SO₄ and concentrated in vacuo to give 4-(dimethoxymethyl)-1-(3-methoxy-4-(4,4,5,5-tetramethyl-1,3,2-dioxaborolan-2-yl)phenyl)piperidine as a yellow oil. The yellow oil was subjected to column chromatography over silica gel (gradient elution: 0-20% EtOAc). LCMS showed 81.5% desired MS. The desired fractions were collected, and concentrated to dryness in vacuo to give 4-(dimethoxymethyl)-1-(3-methoxy-4-(4,4,5,5-tetramethyl-1,3,2-dioxaborolan-2-yl)phenyl)piperidine (378 mg, 787 μmol, 17%, 81.5% purity) as a colorless oil. LC-MS (ESI⁺) m/z: 392.3 (M+H)⁺.

Step 3: A 8 mL thread vial equipped with magnetic stirrer. 6-(benzyloxy)-2-phenyl-3,4-dihydronaphthalen-1-yl trifluoromethanesulfonate (534 mg, 1.2 Eq, 1.16 mmol) was added to a mixture suspension of [1, l′-Bis(diphenylphosphino)ferrocene]dichloropalladium(II) Complex With Dichloromethane (78.9 mg, 0.1 Eq, 96.6 μmol) and 4-(dimethoxymethyl)-1-(3-methoxy-4-(4,4,5,5-tetramethyl-1,3,2-dioxaborolan-2-yl)phenyl)piperidine (378 mg, 1 Eq, 966 μmol) and sodium carbonate (307 mg, 186 μL, 3 Eq, 2.90 mmol) in 1,4-Dioxane (8 mL) and H₂O (2 mL). The mixture was stirred at 90° C. for 16 hour. LCMS showed 41% desired MS. TLC (petroleum ether:ethyl acetate=10:1, UV) showed one main new spot was observed. The reaction was quenched with water (100 mL) and extracted with ethyl acetate (200 mL*2). The organic layer was washed with brine (50 mL) and dried over Na₂SO₄ and concentrated in vacuo to give 1-(4-(6-(benzyloxy)-2-phenyl-3,4-dihydronaphthalen-1-yl)-3-methoxyphenyl)-4-(dimethoxymethyl)piperidine as a black oil. The black oil was subjected to column chromatography over silica gel (gradient elution: 0-15% EtOAc). LCMS showed 97.7% desired MS. The desired fractions were collected, and concentrated to dryness in vacuo to give 1-(4-(6-(benzyloxy)-2-phenyl-3,4-dihydronaphthalen-1-yl)-3-methoxyphenyl)-4-(dimethoxymethyl)piperidine (233 mg, 395 μmol, 40.9%, 97.7% Purity) as a red oil. LC-MS (ESI⁺) m/z: 576.3 (M+H)⁺.

Step 4: A mixture of 1-(4-(6-(benzyloxy)-2-phenyl-3,4-dihydronaphthalen-1-yl)-3-methoxyphenyl)-4-(dimethoxymethyl)piperidine (183 mg, 1 Eq, 318 μmol), Pd/C (200 mg, 5.91 Eq, 1.88 mmol, 10% purity) in MeOH (5 mL) and THF (5 mL) was degassed and purged with H₂ for 3 times, and then the mixture was stirred at 50° C. for 16 hour under H₂ atmosphere (15 psi). LCMS showed 77.8% desired. TLC (petroleum ether:ethyl acetate=5:1, UV) showed one main new spot was observed. The reaction was filtered and concentrated to dryness in vacuo to give a brown oil. The brown oil was subjected to column chromatography over silica gel (gradient elution: 0-20% EtOAc). LCMS showed 95% desired. The desired fractions were collected, and concentrated to dryness in vacuo to give a brown oil (140 mg, 0.27 mmol, 86% yield, 95% purity).

Step 5: The brown oil was purified by SFC. Condition: 0.1% NH₃H₂O EtOH; Column: DAICEL CHIRALCEL OJ (250 mm*30 mm, 10 um), at the beginning: A (60%) and B (40%), at the end; A: (60%) and B (40%), Flow Rate (ml/min) δ0. The pure fractions were collected and the solvent was evaporated under vacuum to give (5R,6R)-5-(4-(4-(dimethoxymethyl)piperidin-1-yl)-2-methoxyphenyl)-6-phenyl-5,6,7,8-tetrahydronaphthalen-2-ol (41 mg, 84 μmol, 51%, 100% purity) as a brown oil and (5S,6S)-5-(4-(4-(dimethoxymethyl)piperidin-1-yl)-2-methoxyphenyl)-6-phenyl-5,6,7,8-tetrahydronaphthalen-2-ol (34 mg, 70 μmol, 42%, 100% Purity) as a brown oil. LC-MS (ESI+) m/z: 488.3 (M+H)⁺.

Step 6: To a solution of (5R,6R)-5-(4-(4-(dimethoxymethyl)piperidin-1-yl)-2-methoxyphenyl)-6-phenyl-5,6,7,8-tetrahydronaphthalen-2-ol (0.15 g, 5 mL, 1 Eq, 0.32 mmol) in THF (5 mL) was added sulfuric acid (31 mg, 1 Eq. 0.32 mmol). The mixture was stirred at 70° C. for 16 hour. LCMS showed 84.5% desired MS. The mixture was adjusted to pH 8-9 by progressively adding NaHCO₃. The reaction mixture was quenched by addition EtOAc (100 mL) at 20° C., and then diluted with H₂O (50 mL) and extracted with EtOAc 50 mL (25 mL*2). The combined organic layers were washed with 10 mL (10 mL*1), dried over Na₂SO₄. The reaction was filtered and concentrated under reduced pressure to give 1-(4-((1R,2R)-6-hydroxy-2-phenyl-1,2,3,4-tetrahydronaphthalen-1-yl)-3-methoxyphenyl)piperidine-4-carbaldehyde (45 mg, 86 μmol, 27%, 84.5% purity) was obtained as a brown oil. LC-MS (ESI+) m/z: 460.3 (M+H)⁺.

Step 7: A mixture of 1-(4-((1R,2R)-6-hydroxy-2-phenyl-1,2,3,4-tetrahydronaphthalen-1-yl)-3-methoxyphenyl)piperidine-4-carbaldehyde (35 mg, 1 Eq, 79 μmol), (S)-3-(1-oxo-5-(piperazin-1-yl)isoindolin-2-yl)piperidine-2,6-dione, PhSO₃H salt (40 mg, 1 Eq, 79 μmol) and sodium acetate (33 mg, 5 Eq, 0.40 mmol) in DCM (1 mL) and MeOH (1 mL) at 20° C. for 0.5 hour, pH=8, then acetic acid (14 mg, 14 μL, 3 Eq, 0.24 mmol) at 20° C. for 1 hour, pH=6, then added sodium triacetoxyhydroborate (34 mg, 2 Eq. 0.16 mmol) the mixture, and then the mixture was stirred at 25° C. for 16 hour. LCMS showed 91.16% desired MS. The reaction was concentrated to give (S)-3-(5-(4-((1-(4-((1R,2R)-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione as a brown oil. The mixture was filtered and the filter cake was washed by EtOAc (50 mL×3). The brown oil was purified by preparative high-performance liquid chromatography. Condition: Column: Xtimate C18 100*30 mm*3 um, A: water(FA), B: CAN, at the beginning: A (85%) and B (15%), at the end: A: (45%) and B (55%), Gradient Time (min) δ; 100% B hold Time (min) 2.5, Flow Rate (ml/min) 30. The pure fractions were collected and the solvent was evaporated under vacuum. The aqueous layer was lyophilized to dryness to give (S)-3-(5-(4-((1-(4-((1R,2R)-6-hydroxy-2-phenyl-1,2,3,4-tetrahydronaphthalen-1-yl)-3-methoxyphenyl)piperidin-4-yl)methyl)piperazin-1-yl)-1-oxoisoindolin-2-yl)piperidine-2,6-dione (24.1 mg, 31.9 μmol, 40%, 99.81% purity) as a white solid.

LCMS: calc. for C₄₆H₅₁N₅O₅: 753.39, found: [M+H]⁺ 754.4.

HPLC: 99.8% purity at 220 nm.

¹H NMR (400 MHZ, DMSO-d₆) δ=ppm 10.95 (s, 1H) 9.07 (s, 1H) 8.16 (s, 0.172H) 7.52 (d, J=8.46 Hz, 1H) 7.04-7.11 (m, 5H) 6.72-6.77 (m, 2H) 6.56 (d, J=8.58 Hz, 2H) 6.46 (dd, J=8.34, 2.38 Hz, 1H) 6.35-6.39 (m, 1H) 6.28-6.33 (m, 1H) 6.12 (d, J=1.79 Hz, 1H) 5.05 (dd, J=13.29, 5.19 Hz, 1H) 4.65 (d, J=5.36 Hz, 1H) 4.30-4.37 (m, 1H) 4.16-4.24 (m, 1H) 3.57 (br s, 2H) 3.29 (br s, 6H) 3.22 (br d, J=14.54 Hz, 2H) 2.92-2.96 (m, 4H) 2.76-2.91 (m, 2H) 2.54-2.62 (m, 3H) 2.27-2.39 (m, 2H) 2.16-2.24 (m, 3H) 1.93-2.00 (m, 1H) 1.77 (br d, J=12.16 Hz, 2H) 1.49-1.68 (m, 2H) 1.17-1.27 (m, 2H)

SFC: retention time, 2.625 min; Area, 91.181%;

### Example 195. Preparation of (I-159) (R)-3-(5-(4-((1-(4-((1R,2R)-2-cyclohexyl-6-hydroxy-1,2,3,4-tetrahydronaphthalen-1-yl)-2,6-difluorophenyl)piperidin-4-yl)methyl)piperazin-1-yl)-1-oxoisoindolin-2-yl)piperidine-2,6-dione

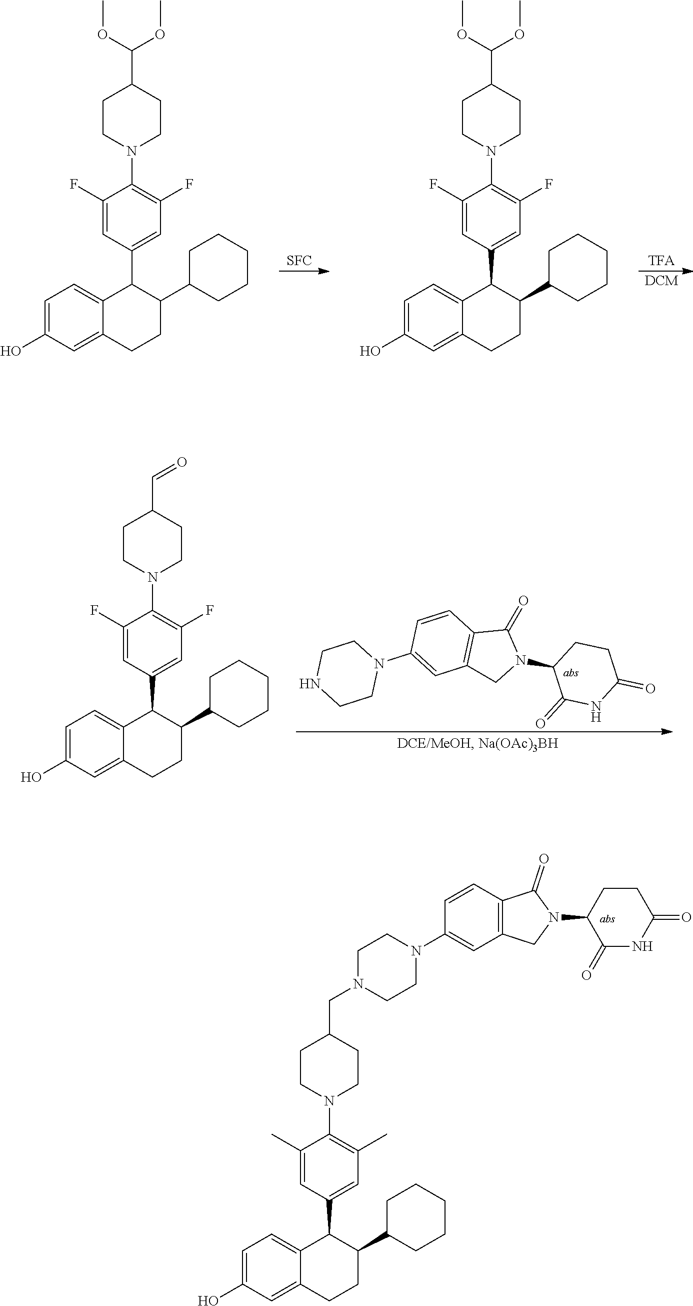

Step 1: 6-cyclohexyl-5-(4-(4-(dimethoxymethyl)piperidin-1-yl)-3,5-difluorophenyl)-5,6,7,8-tetrahydronaphthalen-2-ol (100 mg, 200 μmol, 1.0 eq.) was purified by SFC(Column: DAICEL CHIRALCEL OD-H (250 mm×30 mm, 10 um); Mobile phase: CO₂-EtOH (0.1% NH₃H₂O); from 35% to 35%; Flow rate: 100 mL/min) to give (5R,6R)-6-cyclohexyl-5-(4-(4-(dimethoxymethyl)piperidin-1-yl)-3,5-difluorophenyl)-5,6,7,8-tetrahydronaphthalen-2-ol (40 mg, 80 μmol, 40%) as a white solid. LC-MS (ESI⁺) m/z: 500.4 (M+H)⁺.

Step 2: To a solution of (5R,6R)-6-cyclohexyl-5-(4-(4-(dimethoxymethyl)piperidin-1-yl)-3,5-difluorophenyl)-5,6,7,8-tetrahydronaphthalen-2-ol (40 mg, 80 μmol, 1.0 eq.) in DCM (3 mL) was added TFA (27 mg, 0.24 mmol, 3.0 eq.). The mixture was stirred at 20° C. for 12 hour. LCMS showed the reaction was completed. The mixture was concentrated in vacuo to give crude product. No further purification as it is used for the next step directly. LC-MS (ESI⁺) m/z: 454.2 (M+H)⁺.

Step 3: To a solution of 1-(4-((1R,2R)-2-cyclohexyl-6-hydroxy-1,2,3,4-tetrahydronaphthalen-1-yl)-2,6-difluorophenyl)piperidine-4-carbaldehyde (25 mg, 55 μmol, 1.0 eq.) and (R)-3-(1-oxo-5-(piperazin-1-yl)isoindolin-2-yl)piperidine-2,6-dione (22 mg, 66 μmol, 1.2 eq.) in DCE (3 ml) was added Sodium triacetoxyborohydride (35 mg, 0.17 mmol, 3.0 eq.). The mixture was stirred at 20° C. for 2 hour. LCMS showed the reaction was completed. The crude was purified by prep.HPLC together (Boston Green ODS 150*30 mm*5 um, water(FA)-ACN as a mobile phase, from 20% to 30%, Gradient Time (min): 12, Flow Rate (ml/min): 25) to give (R)-3-(5-(4-((1-(4-((1R,2R)-2-cyclohexyl-6-hydroxy-1,2,3,4-tetrahydronaphthalen-1-yl)-2,6-difluorophenyl)piperidin-4-yl)methyl)piperazin-1-yl)-1-oxoisoindolin-2-yl)piperidine-2,6-dione (20.6 mg, 26.9 μmol, 49%) as a white solid. LC-MS (ESI⁺) m/z: 766.5 (M+H)⁺.

LCMS: calc. for C₄₅H₅₃F₂N₅O₄: 765.95, found: [M+H]⁺ 766.5.

HPLC: 98.84% purity at 220 nm.

¹H NMR (500 MHz, METHANOL-d₄) δ: ppm 7.63 (d, J=8.54 Hz, 1H), 7.05-7.10 (m, 2H), 6.67 (d, J=8.50 Hz, 1H), 6.58 (d, J=2.50 Hz, 1H), 6.48-6.55 (m, 3H), 5.09 (dd, J=13.50, 5.04 Hz, 1H), 4.35-4.44 (m, 2H), 4.15 (d, J=4.50 Hz, 1H), 3.35-3.39 (m, 4H), 3.15-3.19 (m, 2H), 3.03-3.11 (m, 2H), 2.85-2.95 (m, 2H), 2.74-2.82 (m, 2H), 2.59-2.64 (m, 4H), 2.42-2.50 (m, 1H), 2.31 (d, J=7.00 Hz, 2H), 2.09-2.18 (m, 2H), 1.71-1.87 (m, 5H), 1.60-1.69 (m, 3H), 1.51 (dq, J=12.50, 6.50 Hz, 1H), 1.28-1.40 (m, 4H), 1.13-1.20 (m, 1H), 1.04-1.12 (m, 3H), 0.79-0.87 (m, 1H).

### Example 196. Preparation of (I-160) (R)-3-(5-(4-((1-(4-((1S,2S)-2-cyclohexyl-6-hydroxy-1,2,3,4-tetrahydronaphthalen-1-yl)-2,6-difluorophenyl)piperidin-4-yl)methyl)piperazin-1-yl)-1-oxoisoindolin-2-yl)piperidine-2,6-dione

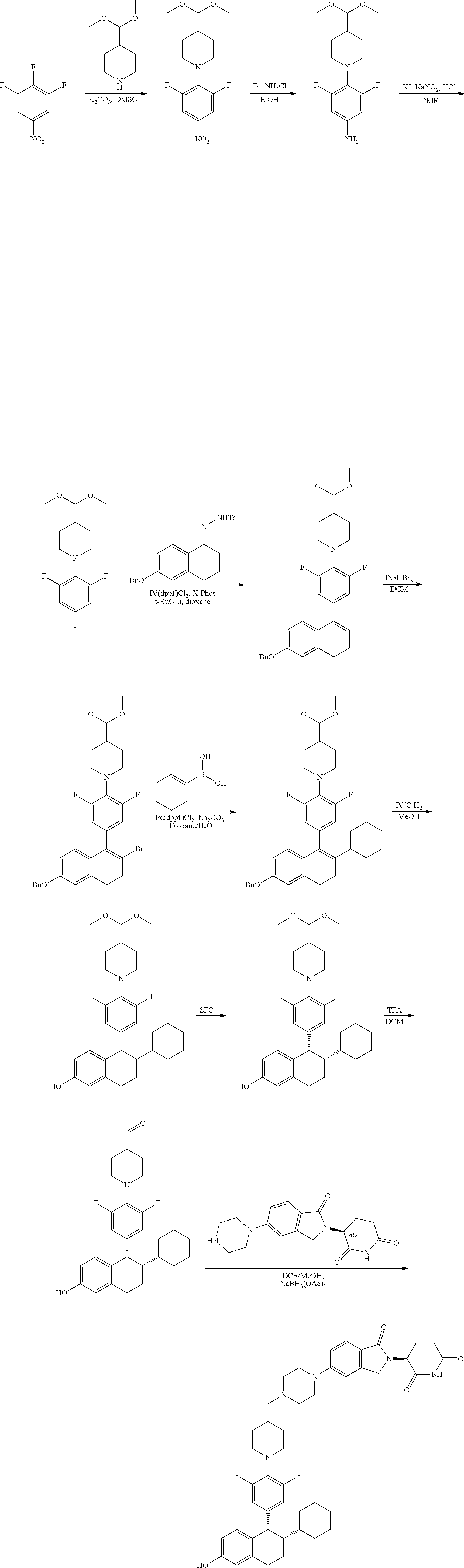

Step 1: To a mixture of 1,2,3-trifluoro-5-nitrobenzene (5.00 g, 28.2 mmol, 1.0 eq.), 4-(dimethoxymethyl)piperidine (4.50 g, 28.2 mmol, 1.0 eq.) and K₂CO₃ (7.80 g, 56.5 mmol, 2.0 eq.) in DMF (20 mL) was added in one portion. The mixture was stirred at 25° C. for 5 min, then heated to 100° C. and stirred for 3 hours. LCMS showed the reaction was completed. The mixture was cooled to 25° C. and concentrated in reduced pressure at 40° C. The residue was poured into ice-water (w/w=1/1) (100 mL) and stirred for 5 min. The aqueous phase was extracted with ethyl acetate (100 mL*2). The combined organic phase was washed with brine (50 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3/1) to afford 1-(2,6-difluoro-4-nitrophenyl)-4-(dimethoxymethyl)piperidine (4.52 g, 13.0 mmol, 46.2%, 91.235% Purity) as yellow solid. LC-MS (ESI⁺) m/z: 317.1 (M+H)⁺.

Step 2: To a mixture of 1-(2,6-difluoro-4-nitrophenyl)-4-(dimethoxymethyl)piperidine (2.00 g, 6.32 mmol, 1.0 eq.), iron (1.06 g, 19.0 mmol, 3.0 eq.) and NH₄Cl (1.01 g, 19.0 mmol, 3.0 eq.) in EtOH (5 mL) and H₂O (5 mL) was added in one portion. The mixture was stirred at 0° C. for 5 min, then heated to 80° C. and stirred for 16 hours. LCMS showed the reaction was completed. The mixture was filter and cooled to 25° C. and concentrated in reduced pressure at 25° C. The residue was poured into ice-water (w/w=1/1) (100 mL) and stirred for 5 min. The aqueous phase was extracted with ethyl acetate (100 mL×2). The combined organic phase was washed with brine (50 mL*1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1) to afford 4-(4-(dimethoxymethyl)piperidin-1-yl)-3,5-difluoroaniline (1.93 g, 5.14 mmol, 81.3%, 76.287% Purity)} as yellow oil. LC-MS (ESI⁺) m/z: 287.1 (M+H)⁺.

Step 3: To a mixture of 4-(4-(dimethoxymethyl)piperidin-1-yl)-3,5-difluoroaniline (2.00 g, 6.99 mmol, 1.0 eq.), potassium iodide (3.48 g, 21.0 mmol, 3.0 eq.) and sodium nitrite (964 mg, 14.0 mmol, 2.0 eq.) in HCl (5 mL) and H₂O (5 mL) was added in one portion. The mixture was added H₂SO₄ (1.37 g, 745 μL, 2 eq., 14.0 mmol) to adjust pH to about 5 and stirred at 0° C. for 1 hour, then heated to 25° C. and stirred for 3 hours. LCMS showed the reaction was completed. The mixture was cooled to 25° C. and concentrated in reduced pressure at 25° C. The residue was poured into ice-water (w/w=1/1) (100 mL) and stirred for 5 min. The aqueous phase was extracted with ethyl acetate (1000 mL*2). The combined organic phase was washed with brine (50 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1) to afford 1-(2,6-difluoro-4-iodophenyl)-4-(dimethoxymethyl)piperidine (1.85 g, 4.11 mmol, 58.8%, 88.192% Purity) as yellow oil. LC-MS (ESI⁺) m/z: 398.0 (M+H)⁺,

Step 4: A mixture of 1-(2,6-difluoro-4-iodophenyl)-4-(dimethoxymethyl)piperidine (1.50 g, 1 eq., 3.78 mmol, 1.0 eq.), Compound 6 (1.91 g, 4.53 mmol, 1.2 eq.), 1,1′-Bis(diphenylphosphino)ferrocene-palladium(II) dichloride (276 mg, 378 μmol, 0.1 eq.), Lithium 2-methyl-2-propanolate (756 mg, 9.44 mmol, 2.5 eq.) and XPhos Pd G3 (160 mg, 0.05 eq., 189 μmol) in Dioxane (40 mL) was degassed and purged with N₂ for 3 times, and then the mixture was stirred at 100° C. for 12 hour under N₂ atmosphere. LCMS showed the reaction was completed. The reaction mixture was quenched by addition EA 10 mL at 20° C., and then diluted with water 30 mL and extracted with EA 30 mL (10 mL*3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=20/1) to give 1-(4-(6-(benzyloxy)-3,4-dihydronaphthalen-1-yl)phenyl)-4-(dimethoxymethyl)-3,5-difluoropiperidine (0.9 g, 2 mmol, 50%) as a yellow solid. LC-MS (ESI⁺) m/z: 506.2 (M+H)⁺.

Step 5: To a solution of 1-(4-(6-(benzyloxy)-3,4-dihydronaphthalen-1-yl)-2,6-difluorophenyl)-4-(dimethoxymethyl)piperidine (1.00 g, 1.98 mmol, 1.0 eq.) in DCM (20 mL) was added Pyridinium bromide perbromide (443 mg, 1.38 mmol, 0.7 eq.). The mixture was stirred at 20° C. for 1 hour. LCMS showed the reaction was completed. The reaction mixture was quenched by addition water 60 mL at 20° C., and then diluted with EA 30 mL and extracted with EA 150 mL (50 mL×3). The combined organic layers were washed with EA 60 mL (20 mL×3), dried over Na₂SO₄, filtered and concentrated under reduced pressure to give a residue. The crude material was purified on silica gel column chromatography (from pure PE to PE/EtOAc=30/1) to give 1-(4-(6-(benzyloxy)-2-bromo-3,4-dihydronaphthalen-1-yl)-2,6-difluorophenyl)-4-(dimethoxymethyl)piperidine (0.6 g, 0.5 mmol, 30%, 50% Purity) as a yellow solid. LC-MS (ESI⁺) m/z: 584.2 (M+H)⁺.

Step 6: A mixture of 1-(4-(6-(benzyloxy)-2-bromo-3,4-dihydronaphthalen-1-yl)-2,6-difluorophenyl)-4-(dimethoxymethyl)piperidine (600 mg, 1.03 mmol, 1.0 eq.), cyclohex-1-en-1-ylboronic acid (154 mg, 1.21 3 mol, 1.2 eq.), PdCl₃(dppf) (75.2 mg, 102.1 μmol, 0.1 eq.) and sodium carbonate (327 mg, 3.08 mmol, 3.0 eq.) in Dioxane (12 mL) and water (3 mL) was degassed and purged with N₂ for 3 times, and then the mixture was stirred at 80° C. for 3 hour under N₂ atmosphere. LCMS showed the reaction was completed. The reaction mixture was quenched by addition EA 30 mL at 20° C., and then diluted with water 30 mL and extracted with EA 90 mL (30 mL×3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=3/1) to give compound 10 (600 mg, 0.31 mmol, 45% yield, 60% Purity) as a yellow solid. LC-MS (ESI⁺) m/z: 586.4 (M+H)⁺.

Step 7: To a solution of 1-(4-(6-(benzyloxy)-2-(cyclohex-1-en-1-yl)-3,4-dihydronaphthalen-1-yl)-2,6-difluorophenyl)-4-(dimethoxymethyl)piperidine (0.400 g, 683 μmol, 1.0 eq.) in MeOH (10 mL) was added Pd/C (10 wt %, 0.2 g) under N₂ atmosphere. The suspension was degassed and purged with H₂ for 3 times. The mixture was stirred under H₂ (50 Psi) at 50° C. for 12 hour. LCMS showed the reaction was completed. The crude was purified by prep. HPLC together (Welch Xtimate C18 150*25 mm*5 um, water(FA)-ACN as a mobile phase, from 85% to 100%, Gradient Time (min): 11, Flow Rate (ml/min): 25) to give 6-cyclohexyl-5-(4-(4-(dimethoxymethyl)piperidin-1-yl)-3,5-difluorophenyl)-5,6,7,8-tetrahydronaphthalen-2-ol (100 mg, 200 μmol, 29.3%) as a white solid. LC-MS (ESI⁺) m/z: 500.4 (M+H)⁺.

Step 8: 6-cyclohexyl-5-(4-(4-(dimethoxymethyl)piperidin-1-yl)-3,5-difluorophenyl)-5,6,7,8-tetrahydronaphthalen-2-ol (100 mg, 200 μmol, 1.0 eq.) was purified by SFC(Column: DAICEL CHIRALCEL OD-H (250 mm*30 mm, 10 um); Mobile phase: CO₂-EtOH (0.1% NH₃H₂O); from 35% to 35%; Flow rate: 100 mL/min) to give (5S,6S)-6-cyclohexyl-5-(4-(4-(dimethoxymethyl)piperidin-1-yl)-3,5-difluorophenyl)-5,6,7,8-tetrahydronaphthalen-2-ol (40 mg, 80 μmol, 40% yield) as a white solid. LC-MS (ESI⁺) m/z: 500.4 (M+H)⁺.

Step 9: To a solution of (5S,6S)-6-cyclohexyl-5-(4-(4-(dimethoxymethyl)piperidin-1-yl)-3,5-difluorophenyl)-5,6,7,8-tetrahydronaphthalen-2-ol (50 mg, 0.10 mmol, 1.0 eq.) in DCM (3 mL) was added TFA (34 mg, 23 μL, 3 eq., 0.30 mmol). The mixture was stirred at 20° C. for 12 hour. LCMS showed the reaction was completed. The mixture was concentrated in vacuo to give crude product. No further purification as it is used for the next step directly. LC-MS (ESI⁺) m/z: 454.2 (M+H)⁺.

Step 10: To a solution of 1-(4-((1S,2S)-2-cyclohexyl-6-hydroxy-1,2,3,4-tetrahydronaphthalen-1-yl)-2,6-difluorophenyl)piperidine-4-carbaldehyde (30 mg, 66 μmol, 1.0 eq.) and (R)-3-(1-oxo-5-(piperazin-1-yl)isoindolin-2-yl)piperidine-2,6-dione (26 mg, 79 μmol, 1.2 eq.) in DCE (3 mL) was added sodium triacetoxyhydroborate (42 mg, 0.20 mmol, 3.0 eq.). The mixture was stirred at 20° C. for 2 hour. LCMS showed the reaction was completed. The crude was purified by prep.HPLC together (Boston Green ODS 150*30 mm*5 um, water(FA)-ACN as a mobile phase, from 20% to 30%, Gradient Time (min): 12, Flow Rate (ml/min): 25) to give (R)-3-(5-(4-((1-(4-((1S,2S)-2-cyclohexyl-6-hydroxy-1,2,3,4-tetrahydronaphthalen-1-yl)-2,6-difluorophenyl)piperidin-4-yl)methyl)piperazin-1-yl)-1-oxoisoindolin-2-yl)piperidine-2,6-dione (23.3 mg, 30.4 μmol, 46%) as a white solid. LC-MS (ESI⁺) m/z: 766.7 (M+H)⁺.

LCMS: calc. for C₄₅H₅₃F₂N₅O₄: 765.95, found: [M+H]⁺ 766.7.

HPLC: 94.59% purity at 220 nm.

¹H NMR (400 MHZ, METHANOL-d₄) δ: ppm 8.44-8.50 (m, 1H), 7.66 (d, J=8.40 Hz, 1H), 7.07-7.14 (m, 2H), 6.69 (d, J=8.40 Hz, 1H), 6.50-6.61 (m, 4H), 5.12 (dd, J=13.20, 5.07 Hz, 1H), 4.37-4.47 (m, 2H), 4.18 (d, J=4.40 Hz, 1H), 3.41 (br d, J=4.80 Hz, 4H), 3.16-3.22 (m, 2H), 3.05-3.14 (m, 2H), 2.87-2.98 (m, 2H), 2.77-2.85 (m, 2H), 2.70-2.76 (m, 4H), 2.43 (br d, J=6.20 Hz, 3H), 2.10-2.20 (m, 2H), 1.75-1.88 (m, 5H), 1.60-1.71 (m, 3H), 1.52 (dq, J=12.80, 6.54 Hz, 1H), 1.26-1.44 (m, 4H), 1.15-1.23 (m, 1H), 1.05-1.14 (m, 3H), 0.79-0.91 (m, 1H).

### Example 197. Preparation of (I-236) (S)-3-(5-(4-((1-(4-((1S,2S)-2-cyclohexyl-6-hydroxy-1,2,3,4-tetrahydronaphthalen-1-yl)-2-fluorophenyl)piperidin-4-yl)methyl)piperazin-1-yl)-1-oxoisoindolin-2-yl)piperidine-2,6-dione and (I-162) (S)-3-(5-(4-((1-(4-((1R,2R)-2-cyclohexyl-6-hydroxy-1,2,3,4-tetrahydronaphthalen-1-yl)-2-fluorophenyl)piperidin-4-yl)methyl)piperazin-1-yl)-1-oxoisoindolin-2-yl)piperidine-2,6-dione

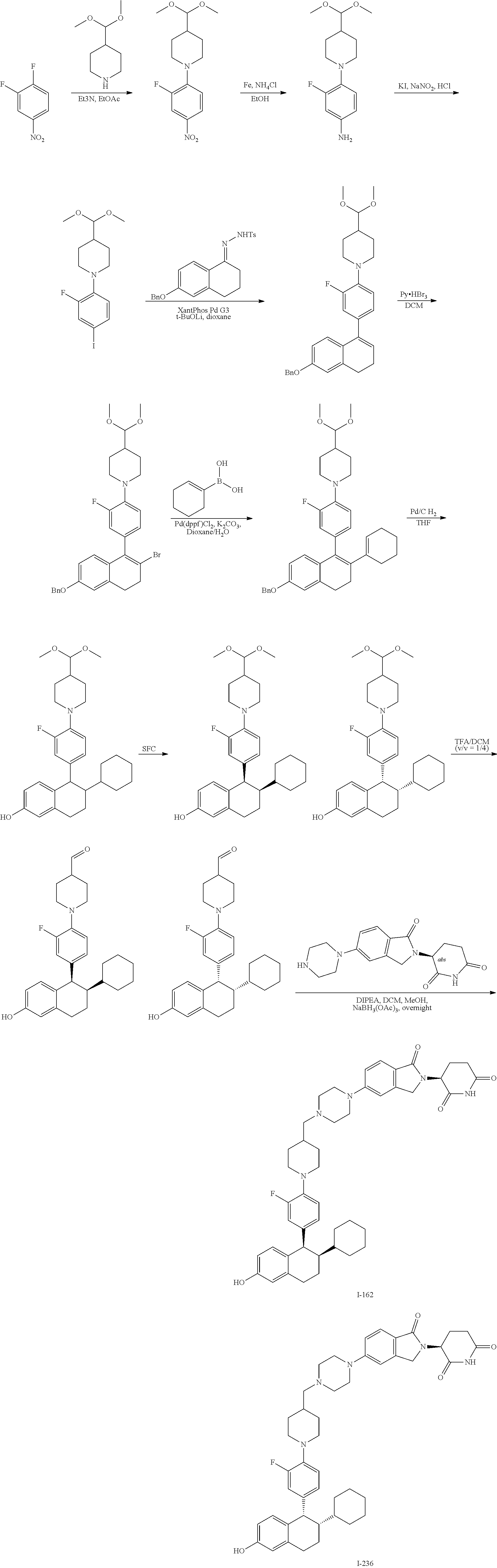

Step 1: To a solution of 1,2-difluoro-4-nitrobenzene (3.0 g, 18.86 mmol, 2.08 mL, 1 eq, 1) and 4-(dimethoxymethyl)piperidine (3.60 g, 22.63 mmol, 1.2 eq) in EtOAc (30 mL) was added Et₃N (5.72 g, 56.57 mmol, 7.87 mL, 3 eq). The mixture was stirred at 25° C. for 3 hr. The mixture was concentrated in reduced pressure. The residue was poured into water (30 mL). The aqueous phase was extracted with ethyl acetate (60 mL*3). The combined organic phase was washed with brine (30 mL*3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 4-(dimethoxymethyl)-1-(2-fluoro-4-nitrophenyl)piperidine (4.77 g, 15.99 mmol, 84.80% yield) as yellow oil. LC-MS (ESI+) m/z: 299.2 (M+H)⁺.

Step 2: To a solution of 4-(dimethoxymethyl)-1-(2-fluoro-4-nitrophenyl)piperidine (3.5 g, 11.73 mmol, 1 eq) in EtOH (40 mL) and H₂O (15 mL) was added Fe (2.62 g, 46.93 mmol, 4 eq) and NH₄Cl (1.26 g, 23.47 mmol, 2 eq). The mixture was stirred at 80° C. for 5 hr. LCMS showed that the starting material was consumed and the desired product was detected. The residue was poured into water (55 mL). The aqueous phase was extracted with ethyl acetate (100 mL*3). The combined organic phase was washed with brine (55 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 4-(4-(dimethoxymethyl)piperidin-1-yl)-3-fluoroaniline (2.96 g, 11.03 mmol, 94.02% yield) as yellow oil. The reaction mixture was filtered under reduced pressure to give a compound. 4-(4-(dimethoxymethyl)piperidin-1-yl)-3-fluoroaniline (2.96 g, 11.03 mmol, 94.02% yield) was obtained as yellow oil.

LC-MS (ESI+) m/z: 269.2 (M+H)⁺.

Step 3: To a solution of 4-(4-(dimethoxymethyl)piperidin-1-yl)-3-fluoroaniline (2.96 g, 11.03 mmol, 1 eq) in MeCN (35 mL) and H₂O (35 mL) was added KI (5.49 g, 33.09 mmol, 3 eq), NaNO₂ (2.28 g, 33.09 mmol, 3 eq) and HCl (1 M, 11.03 mL, leg). The mixture was stirred at 25° C. for 5 hr. LC-MS showed that the starting material was consumed and the desired product was detected. The residue was poured into water (35 mL). The aqueous phase was extracted with ethyl acetate (70 mL*3). The combined organic phase was washed with brine (35 mL*3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1:1) to afford 5 (1.5 g, 3.96 mmol, 35.86% yield) as yellow oil. The reaction mixture was filtered under reduced pressure to give a compound. 4-(dimethoxymethyl)-1-(2-fluoro-4-iodophenyl)piperidine (1.5 g, 3.96 mmol, 35.86% yield) was obtained as yellow oil. LC-MS (ESI+) m/z: 380.1 (M+H)⁺.

Step 4; A mixture of 4-(dimethoxymethyl)-1-(2-fluoro-4-iodophenyl)piperidine (1.6 g, 4.22 mmol, 1 eq), (E)-N′-(6-(benzyloxy)-3,4-dihydronaphthalen-1 (2H)-ylidene)-4-methylbenzenesulfonohydrazide (1.95 g, 4.64 mmol, 1.1 eq), t-BuOLi (1.01 g, 12.66 mmol, 1.14 mL, 3 eq) and XantPhos-Pd-G3 (705.78 mg, 843.86 μmol, 0.2 eq) in dioxane (30 mL) was degassed and purged with N₂ for 3 times, and then the mixture was stirred at 100° C. for 16 hr under N₂ atmosphere. LCMS showed that the starting material was consumed and the desired product was detected. The residue was poured into water (30 mL). The aqueous phase was extracted with ethyl acetate (60 mL*3). The combined organic phase was washed with brine (30 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 1-(4-(6-(benzyloxy)-3,4-dihydronaphthalen-1-yl)-2-fluorophenyl)-4-(dimethoxymethyl)piperidine (1.2 g, 2.46 mmol, 58.33% yield) as yellow oil. The reaction mixture was filtered under reduced pressure to give a compound. 1-(4-(6-(benzyloxy)-3,4-dihydronaphthalen-1-yl)-2-fluorophenyl)-4-(dimethoxymethyl)piperidine (1.2 g, 2.46 mmol, 58.33% yield) was obtained as yellow oil.

LC-MS (ESI+) m/z: 488.3 (M+H)⁺.

Step 5: To a solution of 1-(4-(6-(benzyloxy)-3,4-dihydronaphthalen-1-yl)-2-fluorophenyl)-4-(dimethoxymethyl)piperidine (500 mg, 1.03 mmol, 1 eq) in DCM (35 mL) was added Py·HBr₃ (327.95 mg, 1.03 mmol, 1 eq). The mixture was stirred at 25° C. for 1 hr. LCMS showed that the starting material was consumed and the desired product was detected. The residue was poured into water (35 mL). The aqueous phase was extracted with ethyl acetate (70 mL*3). The combined organic phase was washed with brine (35 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 8 (400 mg, 706.09 μmol, 68.86% yield) as yellow oil. The reaction mixture was filtered under reduced pressure to give a compound. 1-(4-(6-(benzyloxy)-2-bromo-3,4-dihydronaphthalen-1-yl)-2-fluorophenyl)-4-(dimethoxymethyl)piperidine (400 mg, 706.09 μmol, 68.86% yield) was obtained as yellow oil. LC-MS (ESI+) m/z: 568.3 (M+H)⁺.

Step 6: A mixture of 1-(4-(6-(benzyloxy)-2-bromo-3,4-dihydronaphthalen-1-yl)-2-fluorophenyl)-4-(dimethoxymethyl)piperidine (952 mg, 1.68 mmol, 1 eq), cyclohex-1-en-1-ylboronic acid (211.68 mg, 1.68 mmol, 1 eq), Pd(dppf)Cl₂ (122.96 mg, 168.05 μmol, 0.1 eq), K₂CO₃ (464.51 mg, 3.36 mmol, 2 eq) in dioxane (20 mL) and H₂O (7 mL) was degassed and purged with N₂ for 3 times, and then the mixture was stirred at 80° C. for 16 hr under N₂ atmosphere. LCMS showed that the starting material was consumed and the desired product was detected. The reaction mixture was filtered under reduced pressure to give a residue. The residue was poured into H₂O (30 mL). The aqueous phase was extracted with ethyl acetate (60 mL*3). The combined organic phase was washed with brine (30 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford 1-(4-(6-(benzyloxy)-2-(cyclohex-1-en-1-yl)-3,4-dihydronaphthalen-1-yl)-2-fluorophenyl)-4-(dimethoxymethyl)piperidine (701 mg, 1.23 mmol, 73.47% yield) as yellow oil. LC-MS (ESI+) m/z: 568.4 (M+H)⁺.

Step 7: To a solution of 1-(4-(6-(benzyloxy)-2-(cyclohex-1-en-1-yl)-3,4-dihydronaphthalen-1-yl)-2-fluorophenyl)-4-(dimethoxymethyl)piperidine (792 mg, 1.40 mmol, 1 eq) in THF (10 mL) was added Pd/C 742.29 mg, 697.51 μmol, 10% purity, 0.5 eq). The mixture was stirred under H₂ at 50° C. and 50 Psi for 16 hr. LCMS showed that the starting material was consumed and the desired product was detected. The reaction mixture was filtered under reduced pressure to give racemic (5R,6R)-6-cyclohexyl-5-(4-(4-(dimethoxymethyl)piperidin-1-yl)-3-fluorophenyl)-5,6,7,8-tetrahydronaphthalen-2-ol. (5R,6R)-6-cyclohexyl-5-(4-(4-(dimethoxymethyl)piperidin-1-yl)-3-fluorophenyl)-5,6,7,8-tetrahydronaphthalen-2-ol (500 mg, 1.04 mmol, 74.42% yield) was obtained as oil. LC-MS (ESI+) m/z: 482.3 (M+H)⁺.

Step 8: (5R,6S)-6-cyclohexyl-5-(4-(4-(dimethoxymethyl)piperidin-1-yl)-3-fluorophenyl)-5,6,7,8-tetrahydronaphthalen-2-ol and (5R,6R)-6-cyclohexyl-5-(4-(4-(dimethoxymethyl)piperidin-1-yl)-3-fluorophenyl)-5,6,7,8-tetrahydronaphthalen-2-ol (500 mg) was separated by chiral SFC(Column DAICEL CHIRALPAK OD (250 mm*30 mm, 10 um), Condition CO₂-EtOH (0.1% NH₃H₂O) Begin B 35% End B 35%) FlowRate (ml/min): 80) (5S,6S)-6-cyclohexyl-5-(4-(4-(dimethoxymethyl)piperidin-1-yl)-3-fluorophenyl)-5,6,7,8-tetrahydronaphthalen-2-ol (160 mg, 332.20 μmol, 32.00% yield), (5R,6R)-6-cyclohexyl-5-(4-(4-(dimethoxymethyl)piperidin-1-yl)-3-fluorophenyl)-5,6,7,8-tetrahydronaphthalen-2-ol (160 mg, 332.20 μmol, 32.00% yield).

Step 9: 1-(4-((1S,2S)-2-cyclohexyl-6-hydroxy-1,2,3,4-tetrahydronaphthalen-1-yl)-2-fluorophenyl)piperidine-4-carbaldehyde: To a solution of (5R,6R)-6-cyclohexyl-5-(4-(4-(dimethoxymethyl)piperidin-1-yl)-3-fluorophenyl)-5,6,7,8-tetrahydronaphthalen-2-ol (273 mg, 566.81 μmol, 1 eq) in DCM (9 mL) was added TFA (64.63 mg, 566.81 μmol, 42.10 μL, 1 eq). The mixture was stirred at 25° C. for 2 hr. LCMS showed that the starting material was consumed and the desired product was detected. The reaction mixture was filtered under reduced pressure to give a residue. 1-(4-((1S,2S)-2-cyclohexyl-6-hydroxy-1,2,3,4-tetrahydronaphthalen-1-yl)-2-fluorophenyl)piperidine-4-carbaldehyde (160 mg, 367.33 μmol, 64.81% yield) was obtained as a solid. LC-MS (ESI+) m/z: 436.3 (M+H)+.

1-(4-((1R,2R)-2-cyclohexyl-6-hydroxy-1,2,3,4-tetrahydronaphthalen-1-yl)-2-fluorophenyl)piperidine-4-carbaldehyde: To a solution of (5S,6S)-6-cyclohexyl-5-(4-(4-(dimethoxymethyl)piperidin-1-yl)-3-fluorophenyl)-5,6,7,8-tetrahydronaphthalen-2-ol (165 mg, 342.58 μmol, 1 eq) in DCM (6 mL) was added TFA 39.06 mg, 342.58 μmol, 25.45 μL, leg). The mixture was stirred at 25° C. for 2 hr. LCMS showed that the starting material was consumed and the desired product was detected. The reaction mixture was filtered under reduced pressure to give a residue. 1-(4-((1R,2R)-2-cyclohexyl-6-hydroxy-1,2,3,4-tetrahydronaphthalen-1-yl)-2-fluorophenyl)piperidine-4-carbaldehyde (101.2 mg, 232.34 μmol, 67.82% yield) was obtained as a solid. LC-MS (ESI+) m/z: 436.4 (M+H)⁺.

Step 10: To a solution of 1-(4-((1S,2S)-2-cyclohexyl-6-hydroxy-1,2,3,4-tetrahydronaphthalen-1-yl)-2-fluorophenyl)piperidine-4-carbaldehyde (80 mg, 183.67 μmol, 1 eq) in DCM (8 mL) and MeOH (8 mL) was added (S)-3-(1-oxo-5-(piperazin-1-yl)isoindolin-2-yl)piperidine-2,6-dione (60.31 mg, 183.67 μmol, 1 eq), NaBH₂(OAc)₃ (77.85 mg, 367.33 μmol, 2 eq) and DIPEA (2.37 mg, 18.37 μmol, 3.20 μL, 0.1 eq). The mixture was stirred at 25° C. for 5 hr. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water(FA)-ACN]; B %: 23%-53%, 12 min) to give (S)-3-(5-(4-((1-(4-((1S,2S)-2-cyclohexyl-6-hydroxy-1,2,3,4-tetrahydronaphthalen-1-yl)-2-fluorophenyl)piperidin-4-yl)methyl)piperazin-1-yl)-1-oxoisoindolin-2-yl)piperidine-2,6-dione (66 mg, 88.24 μmol, 48.05% yield) as a white solid. LC-MS (ESI+) m/z: 748.6 (M+H)⁺.

LCMS: calc. for C₄₅H₅₄FN₅O₄: 747.42, found: [M+H]⁺ 748.6.

HPLC: 100% purity at 220 nm.

¹H NMR (400 MHZ, METHANOL-d₄) δ ppm 8.43 (s, 1H) 7.64 (d, J=8.58 Hz, 1H) 7.07-7.12 (m, 2H) 6.89-6.95 (m, 1H) 6.77 (dd, J=8.29, 1.49 Hz, 1H) 6.62-6.68 (m, 2H) 6.56 (d, J=2.50 Hz, 1H) 6.47 (dd, J=8.34, 2.50 Hz, 1H) 5.10 (dd, J=13.23, 5.13 Hz, 1H) 4.35-4.45 (m, 2H) 4.15 (d, J=4.65 Hz, 1H) 3.39 (br d, J=4.65 Hz, 6H) 2.85-2.96 (m, 2H) 2.64-2.81 (m, 8H) 2.40-2.52 (m, 3H) 2.10-2.19 (m, 2H) 1.86-1.93 (m, 2H) 1.74-1.83 (m, 3H) 1.51-1.67 (m, 4H) 1.37-1.47 (m, 2H) 1.23-1.32 (m, 2H) 1.03-1.19 (m, 4H) 0.74-0.86 (m, 1H).

Step 11: To a solution of 1-(4-((1R,2R)-2-cyclohexyl-6-hydroxy-1,2,3,4-tetrahydronaphthalen-1-yl)-2-fluorophenyl)piperidine-4-carbaldehyde (85 mg, 195.15 μmol, 1 eq) in DCM (8 mL) and MeOH (8 mL) was added (S)-3-(1-oxo-5-(piperazin-1-yl)isoindolin-2-yl)piperidine-2,6-dione (64.08 mg, 195.15 μmol, 1 eq), NaBH₃(OAc)₃ (82.72 mg, 390.29 μmol, 2 eq) and DIPEA (2.52 mg, 19.51 μmol, 3.40 μL, 0.1 eq). The mixture was stirred at 25° C. for 5 hr. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water(FA)-ACN]; B %: 23%-53%, 12 min) to give (S)-3-(5-(4-((1-(4-((1R,2R)-2-cyclohexyl-6-hydroxy-1,2,3,4-tetrahydronaphthalen-1-yl)-2-fluorophenyl)piperidin-4-yl)methyl)piperazin-1-yl)-1-oxoisoindolin-2-yl)piperidine-2,6-dione (67 mg, 89.58 μmol, 45.90% yield) was obtained as a white solid. LC-MS (ESI+) m/z: 748.6 (M+H)+.

LCMS: calc. for C₄₅H₅₄FN₅O₄: 747.42, found: [M+H]⁺ 748.6.

HPLC: 96.14% purity at 220 nm ¹H NMR (400 MHZ, METHANOL-d₄) δ ppm 8.46 (s, 1H) 7.66 (d, J=8.46 Hz, 1H) 7.09-7.13 (m, 2H) 6.94 (t, J=8.76 Hz, 1H) 6.77-6.82 (m, 1H) 6.64-6.69 (m, 2H) 6.58 (d, J=2.50 Hz, 1H) 6.47-6.51 (m, 1H) 5.12 (dd, J=13.29, 5.19 Hz, 1H) 4.37-4.50 (m, 2H) 4.15 (s, 1H) 3.42 (br d, J=4.29 Hz, 6H) 2.87-2.98 (m, 2H) 2.67-2.84 (m, 8H) 2.39-2.52 (m, 3H) 2.13-2.22 (m, 2H) 1.88-1.94 (m, 2H) 1.75-1.85 (m, 3H) 1.54-1.69 (m, 4H) 1.40-1.50 (m, 2H) 1.24-1.33 (m, 2H) 1.03-1.19 (m, 4H) 0.89 (br d, J=2.74 Hz, 1H).

### Example 198. Preparation of (I-235) (S)-3-(5-(4-((1-(4-((1R, 2S)-2-cyclohexyl-6-hydroxy-1,2,3,4-tetrahydronaphthalen-1-yl)-3-fluoro-5-methylphenyl)piperidin-4-yl)methyl)piperazin-1-yl)-1-oxoisoindolin-2-yl)piperidine-2,6-dione

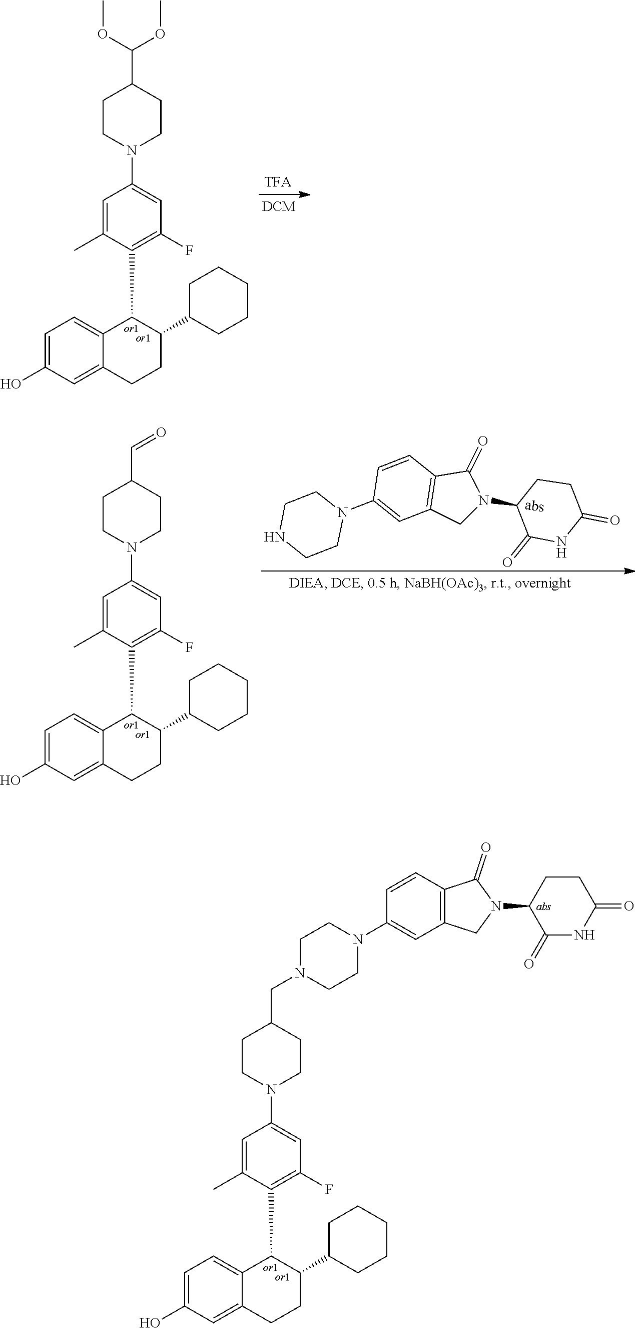 [SMILES: COC(OC)C1CCN(c2cc(C)c([C@H]3c4ccc(O)cc4CC[C@H]3C3CCCCC3)c(F)c2)CC1 ; Cc1cc(N2CCC(C=O)CC2)cc(F)c1[C@H]1c2ccc(O)cc2CC[C@H]1C1CCCCC1 ; O=C1CC[C@H](N2Cc3cc(N4CCNCC4)ccc3C2=O)C(=O)N1 ; Cc1cc(N2CCC(CN3CCN(c4ccc5c(c4)CN([C@H]4CCC(=O)NC4=O)C5=O)CC3)CC2)cc(F)c1[C@H]1c2ccc(O)cc2CC[C@H]1C1CCCCC1]

Step 1: To a solution of (5R,6S)-6-cyclohexyl-5-(4-(4-(dimethoxymethyl)piperidin-1-yl)-2-fluoro-6-methylphenyl)-5,6,7,8-tetrahydronaphthalen-2-ol (150 mg, 302.62 μmol, 1 eq.) in DCM (9 mL) was added TFA (3.45 g, 30.29 mmol, 2.25 mL, 100.09 eq.). The mixture was stirred at 20° C. for 2 hour. LCMS showed the reaction was completed. The mixture was concentrated in vacuum to give crude product. No further purification as it was used for the next step directly. LC-MS (ESI+) m/z: 450.4 (M+H)⁺.

Step 2: A mixture of 1-(4-((1R,2S)-2-cyclohexyl-6-hydroxy-1,2,3,4-tetrahydronaphthalen-1-yl)-3-fluoro-5-methylphenyl)piperidine-4-carbaldehyde (150 mg, 333.63 μmol, 1 eq.), (S)-3-(1-oxo-5-(piperazin-1-yl)isoindolin-2-yl)piperidine-2,6-dione (120.51 mg, 366.99 μmol, 1.1 eq.) and DIEA (86.24 mg, 667.26 μmol, 116.22 μL, 2 eq.) were added in DCE (10 mL). After addition, the mixture was stirred at 25° C. for 30 min, and then NaBH(OAc) 3 (212.13 mg, 1.00 mmol, 3 eq.) was added in the mixture. The reaction mixture was stirred at 25° C. for 16 hour. LCMS showed the reaction was completed. The crude was purified by preparative HPLC(C18-1 150*30 mm*5 um, Condition water (NH₄HCO₃)-CAN as a mobile phase, B %: 60%-90%, 11 min) to give (S)-3-(5-(4-((1-(4-((1R,2S)-2-cyclohexyl-6-hydroxy-1,2,3,4-tetrahydronaphthalen-1-yl)-3-fluoro-5-methylphenyl)piperidin-4-yl)methyl)piperazin-1-yl)-1-oxoisoindolin-2-yl)piperidine-2,6-dione (47.8 mg, 62.73 μmol, 18.80% yield, 100% purity) as a white solid. LC-MS (ESI+) m/z: 762.6 (M+H)⁺.

LCMS: calc. for C₄₆H₅₆FN₅O₄: 761.98, found: [M+H]⁺ 762.6.

HPLC: 100% purity at 220 nm.

¹H NMR: ¹H NMR (400 MHz, METHANOL-d₄) δ ppm 7.65 (1H, d, J=8.46 Hz), 7.03-7.15 (5H, m), 6.76 (1H, s), 6.62 (1H, dd, J=12.34, 1.61 Hz), 5.09 (1H, br s), 4.40 (2H, d, J=5.84 Hz), 3.93-3.98 (1H, m), 3.76 (2H, br d, J=12.28 Hz), 3.38 (4H, br d, J=4.77 Hz), 3.01-3.06 (1H, m), 2.84-2.95 (2H, m), 2.73-2.80 (4H, m), 2.64 (4H, br d, J=4.29 Hz), 2.42-2.51 (2H, m), 2.39 (1H, br t, J=5.07 Hz), 2.34 (2H, br d, J=6.91 Hz), 2.10-2.19 (3H, m), 1.94 (3H, br d, J=13.47 Hz), 1.86 (3H, s), 1.73 (1H, br d, J=13.11 Hz), 1.60-1.66 (4H, m), 1.49 (1H, br dd, J=12.16, 2.98 Hz), 1.36-1.43 (2H, m), 1.23 (2H, br dd, J=13.17, 12.10 Hz), 1.04-1.09 (1H, m).

### Example 199. Preparation of (I-304) (1S,2R)-2-cyclohexyl-1-[4-[4-(dimethoxymethyl)-1-piperidyl]-2-fluoro-6-methyl-phenyl]tetralin-6-ol

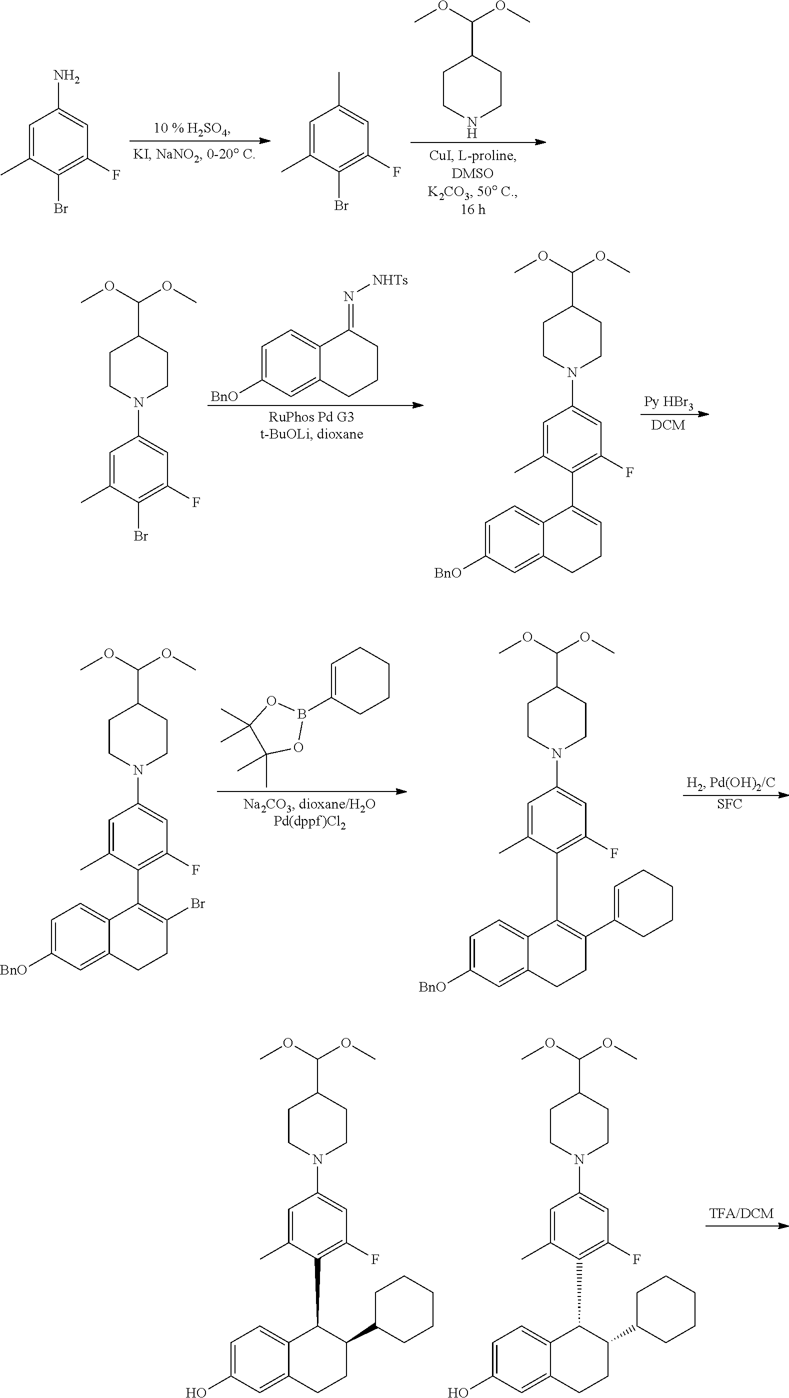

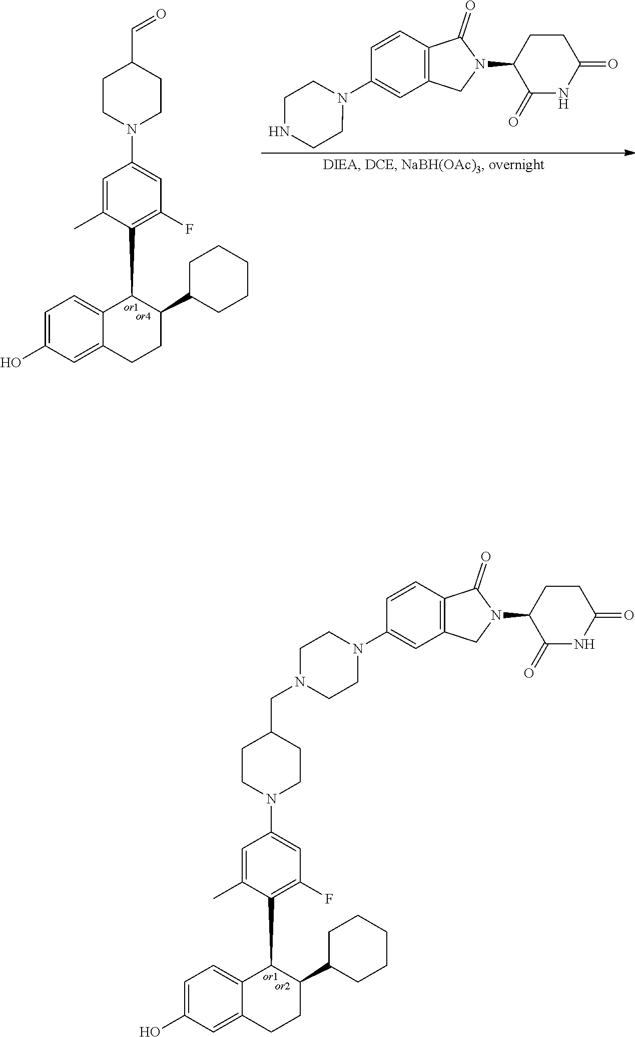 [SMILES: Cc1cc(N2CCC(C=O)CC2)cc(F)c1[C@@H]1c2ccc(O)cc2CC[C@@H]1C1CCCCC1 ; O=C1CC[C@H](N2Cc3cc(N4CCNCC4)ccc3C2=O)C(=O)N1 ; Cc1cc(N2CCC(CN3CCN(c4ccc5c(c4)CN([C@H]4CCC(=O)NC4=O)C5=O)CC3)CC2)cc(F)c1[C@@H]1c2ccc(O)cc2CC[C@@H]1C1CCCCC1]

Step 1: To a solution of 4-bromo-3-fluoro-5-methyl-aniline (9 g, 43.23 mmol) in ACN (130 mL) was added a solution of H₂SO₄ (10.59 g, 107.96 mmol, 5.75 mL) in H₂O (7 mL) at 0° C. After stirring for 5 minutes, a solution of NaNO₂ (5.96 g, 86.39 mmol) in H₂O (7 mL) was added dropwise and the reaction mixture was stirred for an additional 15 minutes at 0° C. KI (28.71 g, 172.97 mmol) in H₂O (14 mL) was then added, and the ice-bath was removed. After warming to 20° C., the reaction was stirred at 20° C. for 20 minutes. The reaction was quenched with sat.aq. Na₂S₂O₃ (100 mL) and extracted with EtOAc (50 mL×3). The combined organic layers were washed with brine (100 mL), dried over Na₂SO₄, filtered and concentrated under reduced pressure to give a residue. The residue was purified by flash silica gel chromatography (ethyl acetate in petroleum ether=0% to 10%) to give the product of 2-bromo-1-fluoro-5-iodo-3-methyl-benzene (12 g, 83.74% yield) as a yellow liquid.

¹H NMR (500 MHz, CDCl₃): δ 7.39 (s, 1H), 7.30 (dd, J=1.5, 7.5 Hz, 1H), 2.39 (s, 3H).

Step 2: A mixture of 2-bromo-1-fluoro-5-iodo-3-methyl-benzene (4.5 g, 13.57 mmol), 4-(dimethoxymethyl)piperidine (2.16 g, 13.57 mmol), CuI (517.07 mg, 2.71 mmol), L-PROLINE (625.15 mg, 5.43 mmol) and K₂CO₃ (3.75 g, 27.15 mmol) in DMSO (40 mL) was degassed and purged with N₂ for 3 times, and then the mixture was stirred at 80° C. for 16 h under N₂ atmosphere. The reaction was quenched with sat.aq. Na₂S₂O₃ (50 mL) and extracted with EtOAc (80 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 10%) to give the product of 1-(4-bromo-3-fluoro-5-methyl-phenyl)-4-(dimethoxymethyl)piperidine (2.7 g, 55.7% yield) as a white solid. LC-MS (ESI⁺) m/z: 348.1 (M+H)⁺,

Step 3: A mixture of 1-(4-bromo-3-fluoro-5-methyl-phenyl)-4-(dimethoxymethyl)piperidine (2.6 g, 7.28 mmol), N-[(E)-(6-benzyloxytetralin-1-ylidene)amino]-4-methyl-benzenesulfonamide (3.37 g, 8.01 mmol), t-BuOLi (1.17 g, 14.57 mmol), RuPhos Pd G3 (609.22 mg, 728.41 μmol) in dioxane (30 mL) was degassed and purged with N₂ for 3 times, and then the mixture was stirred at 110° C. for 16 h under N₂ atmosphere. The reaction was quenched with water (60 mL) and extracted with EtOAc (80 mL×3). The combined organic layers were washed with brine (80 mL), dried over Na₂SO₄, filtered and concentrated under reduced pressure to give a residue. The residue was purified by flash silica gel chromatography (ethyl acetate in petroleum ether=0% to 10%) to give the product of 1-[4-(6-benzyloxy-3,4-dihydronaphthalen-1-yl)-3-fluoro-5-methyl-phenyl]-4-(dimethoxymethyl)piperidine (3 g, 73.9% yield) as a yellow solid. LC-MS (ESI⁺) m/z: 502.4 (M+H)⁺.

Step 4: To a solution of 1-[4-(6-benzyloxy-3,4-dihydronaphthalen-1-yl)-3-fluoro-5-methyl-phenyl]-4-(dimethoxymethyl)piperidine (3 g, 5.38 mmol) in DCM (30 mL) was added BLAH;pyridin-1-ium (1.69 g, 5.27 mmol) at 0° C. The mixture was stirred at 0° C. for 0.5 h. The reaction was quenched with water (50 mL) and extracted with EtOAc (60 mL×3). The combined organic layers were washed with brine (50 mL), dried over Na₂SO₄, filtered and concentrated under reduced pressure to give a residue. The residue was purified by flash silica gel chromatography (ethyl acetate in petroleum ether=0% to 10%) to give the product of 1-[4-(6-benzyloxy-2-bromo-3,4-dihydronaphthalen-1-yl)-3-fluoro-5-methyl-phenyl]-4-(dimethoxymethyl)piperidine (3 g, 89.3% yield) as a yellow solid. LC-MS (ESI⁺) m/z: 580.3 (M+H)⁺.

Step 5: A mixture of 1-[4-(6-benzyloxy-2-bromo-3,4-dihydronaphthalen-1-yl)-3-fluoro-5-methyl-phenyl]-4-(dimethoxymethyl)piperidine (3 g, 4.81 mmol), 2-(cyclohexen-1-yl)-4,4,5,5-tetramethyl-1,3,2-dioxaborolane (1.30 g, 6.25 mmol, 1.34 mL), Pd(dppf)Cl₂ (351.66 mg, 480.60 μmol), Na₂CO₃ (1.02 g, 9.61 mmol, 2 eq) in dioxane (30 mL) and H₂O (6 mL) was degassed and purged with N₂ for 3 times, and then the mixture was stirred at 100° C. for 16 h under N₂ atmosphere. The reaction was quenched with water (60 mL) and extracted with EtOAc (60 mL×3). The combined organic layers were washed with brine (100 mL.), dried over Na₂SO₄, filtered and concentrated under reduced pressure to give a residue. The residue was purified by flash silica gel chromatography (ethyl acetate in petroleum ether=0% to 9%) to give the product of 1-[4-[6-benzyloxy-2-(cyclohexen-1-yl)-3,4-dihydronaphthalen-1-yl]-3-fluoro-5-methyl-phenyl]-4-(dimethoxymethyl)piperidine (1.4 g, 49.5% yield) as a white solid. LC-MS (ESI⁺) m/z: 582.5 (M+H)⁺.

Step 6: To a solution of Pd(OH)₂/C (2 g, 20% purity) in MeOH (20 mL) was added 1-[4-[6-benzyloxy-2-(cyclohexen-1-yl)-3,4-dihydronaphthalen-1-yl]-3-fluoro-5-methyl-phenyl]-4-(dimethoxymethyl)piperidine (1 g, 1.70 mmol) under N₂. The suspension was degassed under vacuum and purged with H₂ several times. The mixture was stirred under H₂ (50 psi) at 80° C. for 16 h. The reaction mixture was filtered and the filter was concentrated. The residue was purified by preparative HPLC (column: Boston Uni C18 40*150*5 um; mobile phase: [water (TFA)-ACN]; gradient: 61%-81% B over 10 min), followed by lyophilization to yield a product which was separated by chiral SFC (column: DAICEL CHIRALCEL OJ (250 mm*30 mm, 10 um); mobile phase: [CO₂-EtOH (0.1% NH₃H₂O)]; B %: 20% %, isocratic elution mode, peak 1: 2.851 min, peak 2:3.037 min) to yield Peak 1 and Peak 2. Peak 1 was concentrated under reduced pressure to give the product of (1S,2R)-2-cyclohexyl-1-[4-[4-(dimethoxymethyl)-1-piperidyl]-2-fluoro-6-methyl-phenyl]tetralin-6-ol (150 mg, 17.8% yield, SFC: Rₜ=2.851) as a white solid. LC-MS (ESI⁺) m/z: 496.5 (M+H)⁺.

### Example 200. Preparation of (I-141) (S)-3-(5-(4-((1-(4-((1R,2R)-2-cyclohexyl-6-hydroxy-1,2,3,4-tetrahydronaphthalen-1-yl)-3-methylphenyl)piperidin-4-yl)methyl)piperazin-1-yl)-1-oxoisoindolin-2-yl)piperidine-2,6-dione

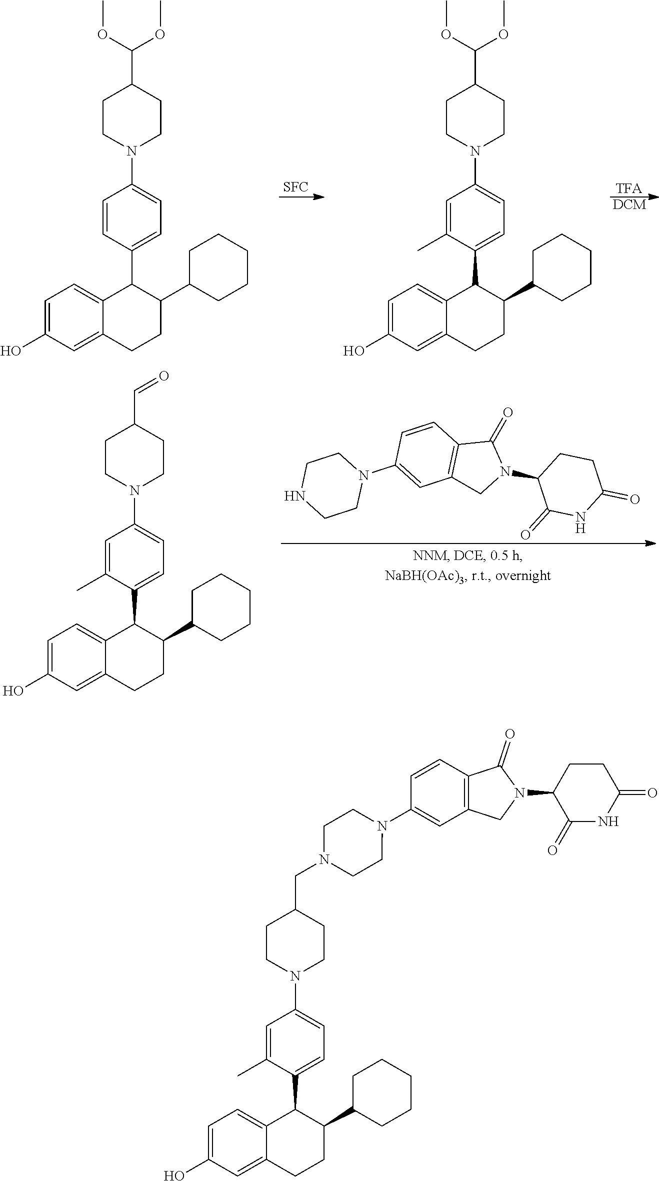

Step 1: 6-cyclohexyl-5-(4-(4-(dimethoxymethyl)piperidin-1-yl)-2-methylphenyl)-5,6,7,8-tetrahydronaphthalen-2-ol (100 mg, 1 eq., 209 μmol) was purified by SFC(Column: DAICEL CHIRALCEL OJ (250 mm*30 mm, 10 um); Mobile phase: 0.1% NH₃H₂O EtOH; from 30% to 30%; Flow rate: 70 mL/min) to give (5R,6R)-6-cyclohexyl-5-(4-(4-(dimethoxymethyl)piperidin-1-yl)-2-methylphenyl)-5,6,7,8-tetrahydronaphthalen-2-ol (40 mg, 84 μmol, 40%) as a white solid.

LC-MS (ESI⁺) m/z: 478.4 (M+H)⁺.

Step 2: To a solution of (5R,6R)-6-cyclohexyl-5-(4-(4-(dimethoxymethyl)piperidin-1-yl)-2-methylphenyl)-5,6,7,8-tetrahydronaphthalen-2-ol (40 mg, 1 eq., 84 μmol) in DCM (3 mL) was added TFA (57 mg, 39 μL, 6 eq., 0.50 mmol). The mixture was stirred at 20° C. for 4 hour. LCMS showed the reaction was completed. The mixture was concentrated in vacuum to give crude product. No further purification as it is used for the next step directly. LC-MS (ESI+) m/z: 432.3 (M+H)⁺.

Step 3: A mixture of 1-(4-((1R,2R)-2-cyclohexyl-6-hydroxy-1,2,3,4-tetrahydronaphthalen-1-yl)-3-methylphenyl)piperidine-4-carbaldehyde (40 mg, 92.68 μmol, 1 eq.), (S)-3-(1-oxo-5-(piperazin-1-yl)isoindolin-2-yl)piperidine-2,6-dione (36.52 mg, 111.21 μmol, 1.2 eq.) and NMM (9.37 mg, 92.68 μmol, 10.19 μL, 1 eq.) were added in DCE (3 mL). After addition, the mixture was stirred at 25° C. for 30 min, and then NaBH(OAc) 3 (49.10 mg, 231.69 μmol, 2.5 eq.) was added in the mixture. The reaction mixture was stirred at 25° C. for 16 hr. LCMS showed the reaction was completed. The crude was purified by preparative HPLC (Welch Xtimate C18 150*25 mm*5 um, Condition water(FA)-CAN as a mobile phase, B %: 35%-65%, 11 min) to give (S)-3-(5-(4-((1-(4-((1R,2R)-2-cyclohexyl-6-hydroxy-1,2,3,4-tetrahydronaphthalen-1-yl)-3-methylphenyl)piperidin-4-yl)methyl)piperazin-1-yl)-1-oxoisoindolin-2-yl)piperidine-2,6-dione (5.5 mg, 7.25 μmol, 7.83% yield, 98.11% purity) as a white solid.

LCMS: calc. for C₄₆H₅₇N₅O₄: 743.44, found: [M+H]⁺ 744.5.

HPLC: 100% purity at 220 nm.

¹H NMR: ¹H NMR (500 MHZ, METHANOL-d₄) δ ppm 8.41-8.50 (1H, m), 7.64 (1H, d, J=8.39 Hz), 7.09 (2H, s), 6.83 (1H, d, J=2.14 Hz), 6.61-6.68 (2H, m), 6.57 (1H, d, J=8.24 Hz), 6.53 (1H, d, J=2.59 Hz), 6.42 (1H, dd, J=8.54, 2.59 Hz), 5.10 (1H, br d, J=8.24 Hz), 4.35-4.46 (3H, m), 3.57-3.66 (2H, m), 3.40 (4H, br s), 2.86-2.95 (2H, m), 2.76-2.81 (2H, m), 2.67-2.74 (4H, m), 2.60-2.66 (2H, m), 2.54 (3H, s), 2.46 (1H, dd, J=13.05, 4.65 Hz), 2.36-2.43 (2H, m), 2.11-2.18 (1H, m), 1.90 (2H, br d, J=12.05 Hz), 1.84 (2H, br d, J=2.90 Hz), 1.69-1.77 (4H, m), 1.55-1.59 (1H, m), 1.43-1.48 (1H, m), 1.33-1.39 (3H, m), 1.21 (2H, br d, J=13.12 Hz), 1.03-1.10 (1H, m), 0.89-0.95 (1H, m), 0.82-0.87 (1H, m), 0.64-0.71 (1H, m).

### Example 201. Preparation of (I-140) (S)-3-(5-(4-((1-(4-((1S,2S)-2-cyclohexyl-6-hydroxy-1,2,3,4-tetrahydronaphthalen-1-yl)-3-methylphenyl)piperidin-4-yl)methyl)piperazin-1-yl)-1-oxoisoindolin-2-yl)piperidine-2,6-dione

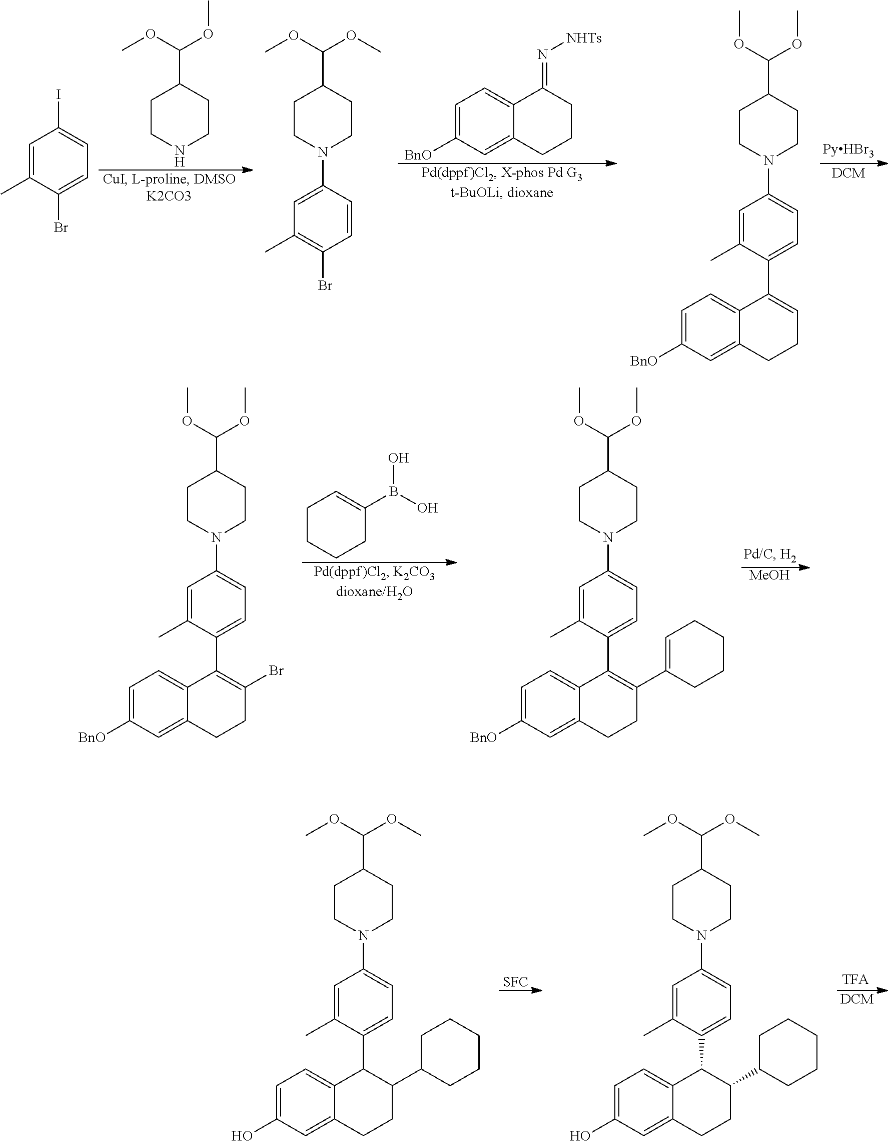

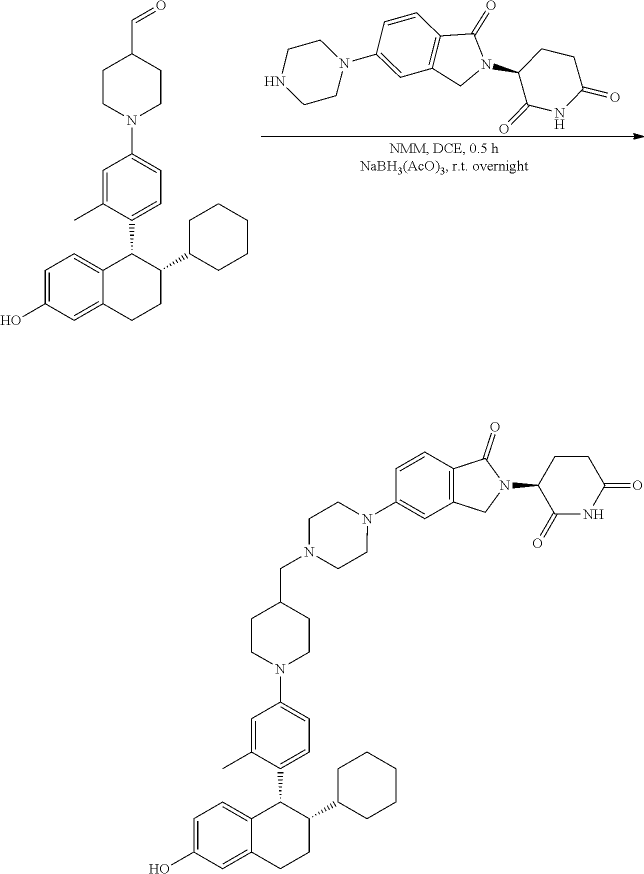 [SMILES: Cc1cc(N2CCC(C=O)CC2)ccc1[C@H]1c2ccc(O)cc2CC[C@H]1C1CCCCC1 ; O=C1CC[C@H](N2Cc3cc(N4CCNCC4)ccc3C2=O)C(=O)N1 ; Cc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1C1CCCCC1]

Step 1: To a mixture of 1-bromo-4-iodo-2-methylbenzene (600 mg, 1 eq, 2.02 mmol), 4-(dimethoxymethyl)piperidine (386 mg, 1.2 eq., 2.42 mmol), (S)-(−)-Proline (116 mg, 86.2 μL, 0.5 eq., 1.01 mmol), K₂CO₃ (838 mg, 3 eq., 6.06 mmol) and, CuI (192 mg, 0.5 eq., 1.01 mmol) in DMSO (2 mL) was added in one portion at 25° C. under N₂. The mixture was stirred at 25° C. for 5 min, then heated to 80° C. and stirred for 12 hours. LCMS showed the reaction was completed. The mixture was cooled to 25° C. and concentrated in reduced pressure at 40° C. The residue was poured into ice-water (50 mL) and stirred for 5 min. The aqueous phase was extracted with ethyl acetate (100 mL×2). The combined organic phase was washed with brine (50 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=5/1) to afford 1-(4-bromo-3-methylphenyl)-4-(dimethoxymethyl)piperidine (423 mg, 1.24 mmol, 61.5%, 96.408% Purity) as yellow solid.

Step 2: A mixture of compound 3 (1.2 g, 1 eq., 3.7 mmol), compound 4 (1.7 g, 1.1 eq., 4.0 mmol), PdCl₂(dppf) (0.27 g, 0.1 eq., 0.37 mmol), X-Phos Pd G3 (85 mg, 0.05 eq., 0.18 mmol) and Lithium 2-methyl-2-propanolate (0.88 g, 1.0 mL, 3 eq., 11 mmol) in Dioxane (100 mL) was degassed and purged with N₂ for 3 times, and then the mixture was stirred at 100° C. for 12 hour under N₂ atmosphere. LCMS showed the reaction was completed. The reaction mixture was quenched by addition EA 30 mL at 20° C., and then diluted with water 30 mL and extracted with EA 90 mL (30 mL×3). The combined organic layers were washed with EA 60 mL (20 mL*3), dried over Na₂SO₄, filtered and concentrated under reduced pressure to give a residue. The crude material was purified on silica gel column chromatography (from pure PE to PE/EA=9/1) to as give 1-(4-(6-(benzyloxy)-3,4-dihydronaphthalen-1-yl)-3-methylphenyl)-4-(dimethoxymethyl)piperidine (1.5 g, 3.1 mmol, 85%) a yellow oil.

LC-MS (ESI+) m/z: 484.2 (M+H⁺).

¹H NMR (400 MHZ, DMSO-d₆) δ: ppm 7.27-7.46 (m, 5H), 6.86-6.95 (m, 2H), 6.73-6.82 (m, 2H), 6.69 (dd, J=8.46, 2.62 Hz, 1H), 6.43 (d, J=8.46 Hz, 1H), 5.70 (t, J=4.47 Hz, 1H), 5.05 (s, 2H), 4.09 (d, J=6.56 Hz, 1H), 4.03 (d, J=7.15 Hz, 1H), 3.70 (d, J=12.16 Hz, 2H), 3.28 (s, 6H), 2.73-2.84 (m, 2H), 2.55-2.66 (m, 2H), 2.22-2.40 (m, 2H), 1.96 (s, 3H), 1.65-1.77 (m, 3H), 1.27-1.39 (m, 2H).

Step 3: To a solution of 1-(4-(6-(benzyloxy)-3,4-dihydronaphthalen-1-yl)-3-methylphenyl)-4-(dimethoxymethyl)piperidine (1.50 g, 1 eq., 3.10 mmol) in DCM (30 mL) was added Pyridinium bromide perbromide (7964 mg, 0.8 eq., 2.48 mmol). The mixture was stirred at 20° C. for 1 hour. LCMS showed the reaction was completed. The reaction mixture was quenched by addition EA 30 mL at 20° C., and then diluted with water 30 mL and extracted with EA 90 mL (30 mL×3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=5/1) to give 1-(4-(6-(benzyloxy)-2-bromo-3,4-dihydronaphthalen-1-yl)-3-methylphenyl)-4-(dimethoxymethyl)piperidine (1.3 g, 2.3 mmol, 75%) as a yellow solid. LC-MS (ESI+) m/z: 562.4.2 (M+H⁺)

Step 4: A mixture of 1-(4-(6-(benzyloxy)-2-bromo-3,4-dihydronaphthalen-1-yl)-3-methylphenyl)-4-(dimethoxymethyl)piperidine (1.300 g, 1 eq., 2.311 mmol), cyclohex-1-en-1-ylboronic acid (320.2 mg, 1.1 eq., 2.542 mmol), potassium carbonate (958.1 mg, 3 eq., 6.933 mmol) and PdCl₂(dppf) (169.1 mg, 0.1 eq., 231.1 μmol) in water (4 mL) and Dioxane (20 mL) was degassed and purged with N₂ for 3 times, and then the mixture was stirred at 80° C. for 12 h under N₂ atmosphere. LCMS showed the reaction was completed. The reaction mixture was quenched by addition EA 30 mL at 20° C., and then diluted with water 30 mL and extracted with EA 90 mL (30 mL×3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=3/1) to give 1-(4-(6-(benzyloxy)-2-(cyclohex-1-en-1-yl)-3,4-dihydronaphthalen-1-yl)-3-methylphenyl)-4-(dimethoxymethyl)piperidine (1.1 g, 1.8 mmol, 76%, 90% Purity) as a yellow solid.

LC-MS (ESI+) m/z: 564.5 (M+H⁺)

¹H NMR (500 MHz, DMSO-d₆) δ: ppm 7.66 (dd, J=9.84, 2.37 Hz, 1H), 7.49-7.55 (m, 1H), 7.44 (dd, J=8, 54, 2.29 Hz, 1H), 7.27 (dd, J=8.54, 2.14 Hz, 1H), 7.04 (t, J=8.62 Hz, 1H), 6.84 (d, J=2.44 Hz, 1H), 6.68 (dd, J=8.47, 2.67 Hz, 1H), 6.53 (dd, J=8.39, 1.22 Hz, 1H), 6.02 (t, J=4.50 Hz, 1H), 3.73 (s, 3H), 2.80 (t, J=8.09 Hz, 2H), 2.37 (td, J=7.82, 4.81 Hz, 2H).

Step 5: To a solution of 1-(4-(6-(benzyloxy)-2-(cyclohex-1-en-1-yl)-3,4-dihydronaphthalen-1-yl)-3-methylphenyl)-4-(dimethoxymethyl)piperidine (1.10 g, 1 eq., 1.95 mmol) in Pd/C (0.60 g, 2.9 eq., 5.6 mmol) was added Pd/C (10%, 0.6 g) under N₂ atmosphere. The suspension was degassed and purged with H₂ for 3 times. The mixture was stirred under H₂ (50 Psi) at 70° C. for 24 hour. LCMS showed the reaction was completed. The crude was purified by prep-HPLC together (Boston Green ODS 150*30 mm*5 um, water(FA)-ACN as a mobile phase, from 65% to 95%, Gradient Time (min): 12, Flow Rate (ml/min): 25) to give 6-cyclohexyl-5-(4-(4-(dimethoxymethyl)piperidin-1-yl)-2-methylphenyl)-5,6,7,8-tetrahydronaphthalen-2-ol (100 mg, 209 μmol, 10.7%).

LC-MS (ESI+) m/z: 478.2 (M+H⁺).

Step 6: 6-cyclohexyl-5-(4-(4-(dimethoxymethyl)piperidin-1-yl)-2-methylphenyl)-5,6,7,8-tetrahydronaphthalen-2-ol (100 mg, 1 eq., 209 μmol) was purified by SFC(Column: DAICEL CHIRALCEL OJ (250 mm*30 mm, 10 um); Mobile phase: 0.1% NH₃H₂O ETOH; from 30% to 30%; Flow rate: 70 mL/min) to give (5S,6S)-6-cyclohexyl-5-(4-(4-(dimethoxymethyl)piperidin-1-yl)-2-methylphenyl)-5,6,7,8-tetrahydronaphthalen-2-ol (40 mg, 84 μmol, 40%) as white solid.

LC-MS (ESI+) m/z: 478.4 (M+H⁺).

Step 7: To a solution of (5S,6S)-6-cyclohexyl-5-(4-(4-(dimethoxymethyl)piperidin-1-yl)-2-methylphenyl)-5,6,7,8-tetrahydronaphthalen-2-ol (50 mg, 1 eq., 0.10 mmol) in DCM (3 mL) was added TFA (36 mg, 24 μL, 3 eq., 0.31 mmol). The mixture was stirred at 20° C. for 4 hour. LCMS showed the reaction was completed. The mixture was concentrated in vacuo to give crude product. No further purification as it is used for the next step directly. LC-MS (ESI+) m/z: 432.3 (M+H⁺).

Step 8: To a solution of 1-(4-((1S,2S)-2-cyclohexyl-6-hydroxy-1,2,3,4-tetrahydronaphthalen-1-yl)-3-methylphenyl)piperidine-4-carbaldehyde (25.00 mg, 1 eq., 57.92 μmol) and (S)-3-(1-oxo-5-(piperazin-1-yl)isoindolin-2-yl)piperidine-2,6-dione (22.82 mg, 1.2 eq., 69.51 μmol) in DCE (3 mL) was added dropwise 4-methylmorfolin (5.859 mg, 6.4 μL, 1 eq., 57.92 μmol) to pH=7 at 20° C. After addition, the mixture was stirred at this temperature for 0.5 hour, and then Sodium triacetoxyborohydride (36.83 mg, 25.8 μL, 3 eq., 173.8 μmol) was added dropwise at 20° C. The resulting mixture was stirred at 20° C. for 1.5 hour. LCMS showed the reaction was completed. The crude was purified by prep-HPLC together (Welch Xtimate C18 150*25 mm*5 um, water (FA)-ACN as a mobile phase, from 22% to 52%, Gradient Time (min): 11, Flow Rate (ml/min): 25) to give (S)-3-(5-(4-((1-(4-((1S,2S)-2-cyclohexyl-6-hydroxy-1,2,3,4-tetrahydronaphthalen-1-yl)-3-methylphenyl)piperidin-4-yl)methyl)piperazin-1-yl)-1-oxoisoindolin-2-yl)piperidine-2,6-dione (5.4 mg, 7.3 μmol, 13%) as a white solid.

LCMS: calc. for C₄₆H₅₇N₅O₄: 743.44, found: [M+H]⁺ 744.44.

HPLC: 100.00% purity at 220 nm.

¹H NMR (400 MHZ, METHANOL-d₄) δ: ppm 8.38-8.48 (m, 1H), 7.65 (d, J=8.46 Hz, 1H), 7.08-7.13 (m, 2H), 6.83 (d, J=2.15 Hz, 1H), 6.60-6.69 (m, 2H), 6.51-6.59 (m, 2H), 6.38-6.45 (m, 1H), 5.10 (dd, J=13.17, 5.07 Hz, 1H), 4.35-4.46 (m, 3H), 3.57-3.64 (m, 2H), 3.41 (s, 4H), 2.85-2.96 (m, 2H), 2.76-2.82 (m, 2H), 2.74 (d, J=4.29 Hz, 4H), 2.61-2.69 (m, 2H), 2.54 (s, 3H), 2.40-2.50 (m, 3H), 2.11-2.18 (m, 1H), 1.82-1.93 (m, 4H), 1.67-1.78 (m, 4H), 1.54-1.60 (m, 1H), 1.43-1.47 (m, 1H), 1.30-1.39 (m, 3H), 1.21 (d, J=9.42 Hz, 2H), 1.02-1.11 (m, 1H), 0.80-0.96 (m, 2H), 0.60-0.77 (m, 1H).

### Example 202. Preparation of (I-139) (S)-3-(5-(4-((1-(4-((1R,2S)-6-hydroxy-2-phenyl-1,2,3,4-tetrahydronaphthalen-1-yl)-3-methylphenyl)piperidin-4-yl)methyl)piperazin-1-yl)-1-oxoisoindolin-2-yl)piperidine-2,6-dione

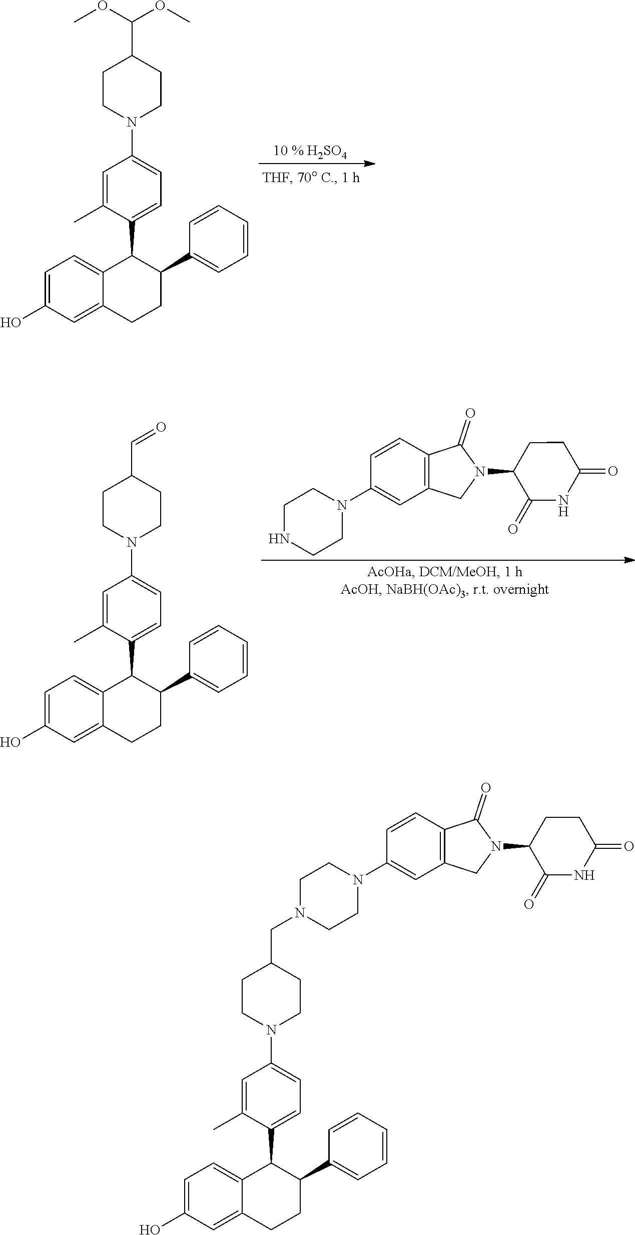 [SMILES: COC(OC)C1CCN(c2ccc([C@@H]3c4ccc(O)cc4CC[C@@H]3c3ccccc3)c(C)c2)CC1 ; Cc1cc(N2CCC(C=O)CC2)ccc1[C@@H]1c2ccc(O)cc2CC[C@@H]1c1ccccc1 ; O=C1CC[C@H](N2Cc3cc(N4CCNCC4)ccc3C2=O)C(=O)N1 ; Cc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1c1ccccc1]

Step 1: To a solution of (5R, 6S)-5-(4-(4-(dimethoxymethyl)piperidin-1-yl)-2-methylphenyl)-6-phenyl-5,6,7,8-tetrahydronaphthalen-2-ol (65 mg, 0.14 mmol) in THF (1 mL) and 10% H₂SO₄ (1 mL) was stirred at 70° C. for 1 h. The reaction mixture was quenched by addition sat. aq. NaHCO₃ (10 mL) at 0° C., then extracted with ethyl acetate (20 mL×2). The combined organic layers were washed with 20 mL brine dried over Na₂SO₄, filtered and concentrated under reduced pressure to give 1-(4-((1R,2S)-6-hydroxy-2-phenyl-1,2,3,4-tetrahydronaphthalen-1-yl)-3-methylphenyl)piperidine-4-carbaldehyde (60 mg, 95% Yield) as a yellow solid, LC-MS (ESI+) m/z: 426.2 (M+H)⁺.

Step 2: A mixture of 1-(4-((1R,2S)-6-hydroxy-2-phenyl-1,2,3,4-tetrahydronaphthalen-1-yl)-3-methylphenyl)piperidine-4-carbaldehyde (55 mg, 0.12 mmol), (S)-3-(1-oxo-5-(piperazin-1-yl)isoindolin-2-yl)piperidine-2,6-dione benzenesulfonate (88 mg, 0.18 mmol) and sodium acetate (49 mg, 0.60 mmol) and acetic acid (14 mg, 0.24 mmol) in THF (2 mL) and MeOH (2 mL) at 25° C. for 1 h, then sodium triacetoxyborohydride (51 mg, 0.24 mmol) was added to the mixture and was stirred at 25° C. for 16 h. The mixture was added H₂O (20 mL), extracted with ethyl acetate (20 mL×2). The combined extracts was dried over anhydrous Na₂SO₄, filtered and concentrated to dryness in vacuo to give a residue. The residue was purified by prep-HPLC (column: Xtimate C18 100*30 mm*3 um; mobile phase: [water(FA)-ACN]; B % 12%-52%, 9 min) to give (S)-3-(5-(4-((1-(4-((1R,2S)-6-hydroxy-2-phenyl-1,2,3,4-tetrahydronaphthalen-1-yl)-3-methylphenyl)piperidin-4-yl)methyl)piperazin-1-yl)-1-oxoisoindolin-2-yl)piperidine-2,6-dione (22.2 mg, 24% Yield) as a white solid. LC-MS (ESI+) m/z: 738.2 (M+H)+.

LCMS: calc. for C₄₆H₅₁N₅O₄: 737.95, found: [M+H]⁺ 738.2.

HPLC: 94.568% purity at 220 nm.

¹HNMR (400 MHZ, DMSO-d₆) δ ppm 10.91-10.98 (m, 1H) 9.01-9.17 (m, 1H) 8.15 (s, 0.567H) 7.52 (d, J=8.70 Hz, 1H) 7.03-7.14 (m, 5H) 6.72 (br d, J=6.79 Hz, 2H) 6.55-6.63 (m, 3H) 6.37-6.50 (m, 3H) 5.05 (dd, J=13.29, 5.19 Hz, 1H) 4.31-4.40 (m, 2H) 4.15-4.25 (m, 1H) 3.54-3.59 (m, 2H) 3.29 (br s, 8H) 2.87-3.04 (m, 4H) 2.59-2.71 (m, 3H) 2.28-2.39 (m, 2H) 2.21 (br d, J=7.03 Hz, 2H) 1.92-2.01 (m, 1H) 1.77 (br d, J=12.16 Hz, 2H) 1.56-1.71 (m, 2H) 1.40 (s, 3H) 1.11-1.24 (m, 2H)

SFC: retention time, 3.211 min; Area, 95.847%;

### Example 203. Preparation of (I-138) (S)-3-(5-(4-((1-(4-((1S,2R)-6-hydroxy-2-phenyl-1,2,3,4-tetrahydronaphthalen-1-yl)-3-methylphenyl)piperidin-4-yl)methyl)piperazin-1-yl)-1-oxoisoindolin-2-yl)piperidine-2,6-dione

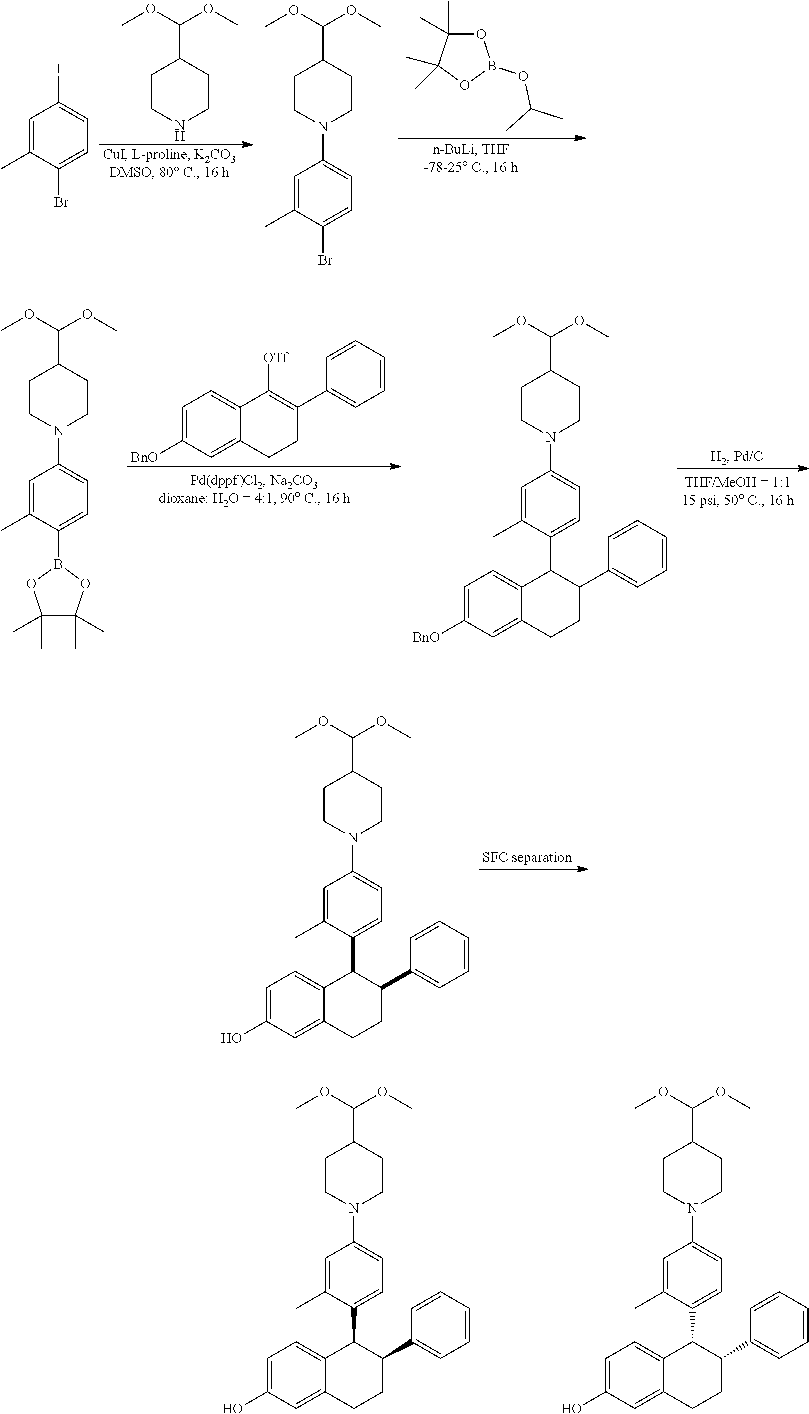

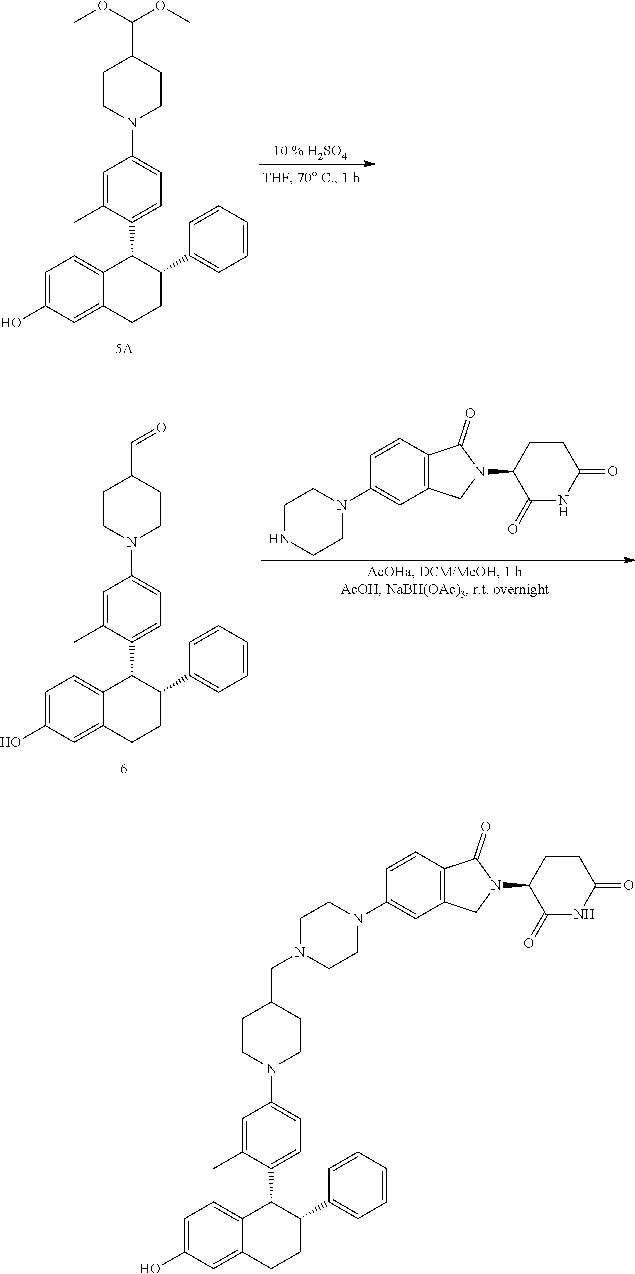 [SMILES: COC(OC)C1CCN(c2ccc([C@H]3c4ccc(O)cc4CC[C@H]3c3ccccc3)c(C)c2)CC1 ; Cc1cc(N2CCC(C=O)CC2)ccc1[C@H]1c2ccc(O)cc2CC[C@H]1c1ccccc1 ; O=C1CC[C@H](N2Cc3cc(N4CCNCC4)ccc3C2=O)C(=O)N1 ; Cc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1c1ccccc1]

Step 1: A mixture of 1-bromo-4-iodo-2-methylbenzene (3.0 g, 10 mmol), 4-(dimethoxymethyl)piperidine (1.6 g, 10 mmol), cuprous iodide (0.38 g, 2.0 mmol), potassium carbonate (2.8 g, 20 mmol) and (S)-(−)-proline (0.47 g, 4.0 mmol) in DMSO (30 mL) at r.t., then the mixture was stirred at 80° C. for 16 h under N₂. The reaction mixture was added H₂O (100 mL) and extracted with ethyl acetate (100 mL×2). Then organic phase was washed with H₂O (100 mL×2) and the combined organic layers was dried over Na₂SO₄, filtered and concentrated under reduced pressure to give a residue. The residue was purified by flash silica gel chromatography (ethyl acetate in petroleum ether=0% to 30%) to give the product of 1-(4-bromo-3-methylphenyl)-4-(dimethoxymethyl)piperidine (1.9 g, 56% Yield) as a yellow solid. LC-MS (ESI⁺) m/z: 330.1 (M+H)⁺.

Step 2: A mixture of 1-(4-bromo-3-methylphenyl)-4-(dimethoxymethyl)piperidine (1.8 g, 5.3 mmol) in THF (50 mL) at −68° C. was added to n-Butyllithium (2.5 M in hexane) (0.37 g, 5.8 mmol) and was stirred for 1 hour, then isopropoxyboronic acid pinacol ester (1.3 g, 6.9 mmol) was added to the reaction and was warmed to 25° C. and stirred for 16 h. The reaction mixture was quenched with aq.NH₄Cl (30 mL), then added H₂O (100 mL) and extracted with ethyl acetate (100 mL×2) and the combined organic layers was dried over Na₂SO₄, filtered and concentrated under reduced pressure to give a residue. The residue was purified by flash silica gel chromatography (ethyl acetate in petroleum ether=0% to 25%) to give the product of 4-(dimethoxymethyl)-1-(3-methyl-4-(4,4,5,5-tetramethyl-1,3,2-dioxaborolan-2-yl)phenyl)piperidine (400 mg, 12% Yield) as a yellow solid. LC-MS (ESI⁺) m/z: 376.5 (M+H)⁺.

Step 3: 6-(benzyloxy)-2-phenyl-3,4-dihydronaphthalen-1-yl trifluoromethanesulfonate (348 mg, 756 μmol) was added to a mixture suspension of [1,1′-Bis(diphenylphosphino)ferrocene]dichloropalladium(II) Complex With Dichloromethane (51.4 mg, 63.0 μmol) and 4-(dimethoxymethyl)-1-(3-methyl-4-(4,4,5,5-tetramethyl-1,3,2-dioxaborolan-2-yl)phenyl)piperidine (380 mg, 630 μmol) and sodium carbonate (200 mg, 1.89 mmol) in 1,4-Dioxane (6 mL) and H₂O (1.5 mL). N₂ was bubbled into the mixture for 5 min. The reaction mixture was heated at 90° C. for 16 h. The reaction mixture was added H₂O (100 mL) and extracted with ethyl acetate (100 mL×2) and the combined organic layers was dried over Na₂SO₄, filtered and concentrated under reduced pressure to give a residue. The residue was purified by flash silica gel chromatography (ethyl acetate in petroleum ether=0% to 25%) to give the product of 1-(4-(6-(benzyloxy)-2-phenyl-3,4-dihydronaphthalen-1-yl)-3-methylphenyl)-4-(dimethoxymethyl)piperidine (340 mg, 92.9% Yield) as a yellow solid. LC-MS (ESI⁺) m/z: 560.2 (M+H)⁺.

Step 4: A mixture of 1-(4-(6-(benzyloxy)-2-phenyl-3,4-dihydronaphthalen-1-yl)-3-methylphenyl)-4-(dimethoxymethyl)piperidine (320 mg, 551 μmol), H₂ (1.11 mg, 551 μmol), Pd/C (500 mg, 470 μmol, 10 wt %) in MeOH (10 mL) was degassed and purged with H₂ for 3 times, and then the mixture was stirred at 50° C. for 16 h under H₂ atmosphere (15 psi). The reaction mixture was filtered and concentrated under reduced pressure to give a residue. The residue was further separated by SFC (condition: column: DAICEL CHIRALPAK AD (250 mm*30 mm, 10 um); mobile phase: [0.1% NH₃·H₂O-lPA]; B %: 45%-45%, min) to give (5S,6R)-5-(4-(4-(dimethoxymethyl)piperidin-1-yl)-2-methylphenyl)-6-phenyl-5,6,7,8-tetrahydronaphthalen-2-ol (65 mg, 25% Yield) was obtained as a white solid. LC-MS (ESI+) m/z: 472.2 (M+H)⁺ and (5R,6S)-5-(4-(4-(dimethoxymethyl)piperidin-1-yl)-2-methylphenyl)-6-phenyl-5,6,7,8-tetrahydronaphthalen-2-ol (65 mg, 25% Yield) was obtained as a white solid. LC-MS (ESI⁺) m/z: 472.1 (M+H)⁺

Step 5: To a solution of (5S,6R)-5-(4-(4-(dimethoxymethyl)piperidin-1-yl)-2-methylphenyl)-6-phenyl-5,6,7,8-tetrahydronaphthalen-2-ol (65 mg, 0.14 mmol) in THF (1 mL) and 10% H₂SO₄ (1 mL) was stirred at 70° C. for 1 h. The reaction mixture was quenched by addition sta. aq. NaHCO₃ (10 mL) at 0° C., then extracted with ethyl acetate (20 mL×2). The combined organic layers were washed with 20 mL brine dried over Na₂SO₄, filtered and concentrated under reduced pressure to give 1-(4-((1S,2R)-6-hydroxy-2-phenyl-1,2,3,4-tetrahydronaphthalen-1-yl)-3-methylphenyl)piperidine-4-carbaldehyde (60 mg, 99% Yield) as a yellow solid. LC-MS (ESI⁺) m/z: 426.1 (M+H)⁺.

Step 6: A mixture of 1-(4-((1S,2R)-6-hydroxy-2-phenyl-1,2,3,4-tetrahydronaphthalen-1-yl)-3-methylphenyl)piperidine-4-carbaldehyde (50 mg, 0.11 mmol), (S)-3-(1-oxo-5-(piperazin-1-yl)isoindolin-2-yl)piperidine-2,6-dione benzenesulfonate (83 mg, 0.17 mmol) and Sodium acetate (47 mg, 0.57 mmol) and Acetic acid (14 mg, 0.23 mmol) in THF (2 mL) and MeOH (2 mL) at 25° C. for 1 h, then Sodium triacetoxyborohydride (48 mg, 0.23 mmol) was added to the mixture and stirred at 25° C. for 16 h. The mixture was added H₂O (20 mL), extracted with ethyl acetate (20 mL×2). The combined extracts was dried over anhydrous Na₂SO₄, filtered and concentrated to dryness in vacuo to give a residue. The residue was purified by prep-HPLC (column: Xtimate C18 100*30 mm*3 um; mobile phase: [water(FA)-ACN]; B % 12%-52%, 8 min) to give (S)-3-(5-(4-((1-(4-((1S,2R)-6-hydroxy-2-phenyl-1,2,3,4-tetrahydronaphthalen-1-yl)-3-methylphenyl)piperidin-4-yl)methyl)piperazin-1-yl)-1-oxoisoindolin-2-yl)piperidine-2,6-dione (15.4 mg, 18% Yield) as a white solid. LC-MS (ESI⁺) m/z: 738.3 (M+H)⁺.

LCMS; calc. for C₄₆H₅₁N₅O₄: 737.95, found: [M+H]⁺ 738.3.

HPLC: 96.133% purity at 220 nm.

¹HNMR (400 MHZ, DMSO-d₆) δ ppm 10.96 (s, 1H) 9.01-9.15 (m, 1H) 8.17 (s, 0.404H) 7.52 (d, J=8.70 Hz, 1H) 7.01-7.15 (m, 5H) 6.72 (br d, J=6.68 Hz, 2H) 6.54-6.65 (m, 3H) 6.37-6.51 (m, 3H) 4.99-5.10 (m, 1H) 4.30-4.42 (m, 2H) 4.16-4.26 (m, 1H) 3.57 (br d, J=8.94 Hz, 2H) 3.29 (br d, J=3.46 Hz, 8H) 2.85-3.05 (m, 4H) 2.56-2.62 (m, 3H) 2.30-2.40 (m, 2H) 2.21 (br d, J=7.15 Hz, 2H) 1.90-2.01 (m, 1H) 1.77 (br d, J=11.92 Hz, 2H) 1.56-1.68 (m, 2H) 1.40 (s, 3H) 1.12-1.23 (m, 2H)

SFC: retention time, 3.030 min; Area, 96.011%;

### Example 204. Preparation of (I-137) (S)-3-(5-(4-((1-(4-((1S,2R)-2-cyclohexyl-6-hydroxy-1,2,3,4-tetrahydronaphthalen-1-yl)-3-fluorophenyl)piperidin-4-yl)methyl)piperazin-1-yl)-1-oxoisoindolin-2-yl)piperidine-2,6-dione

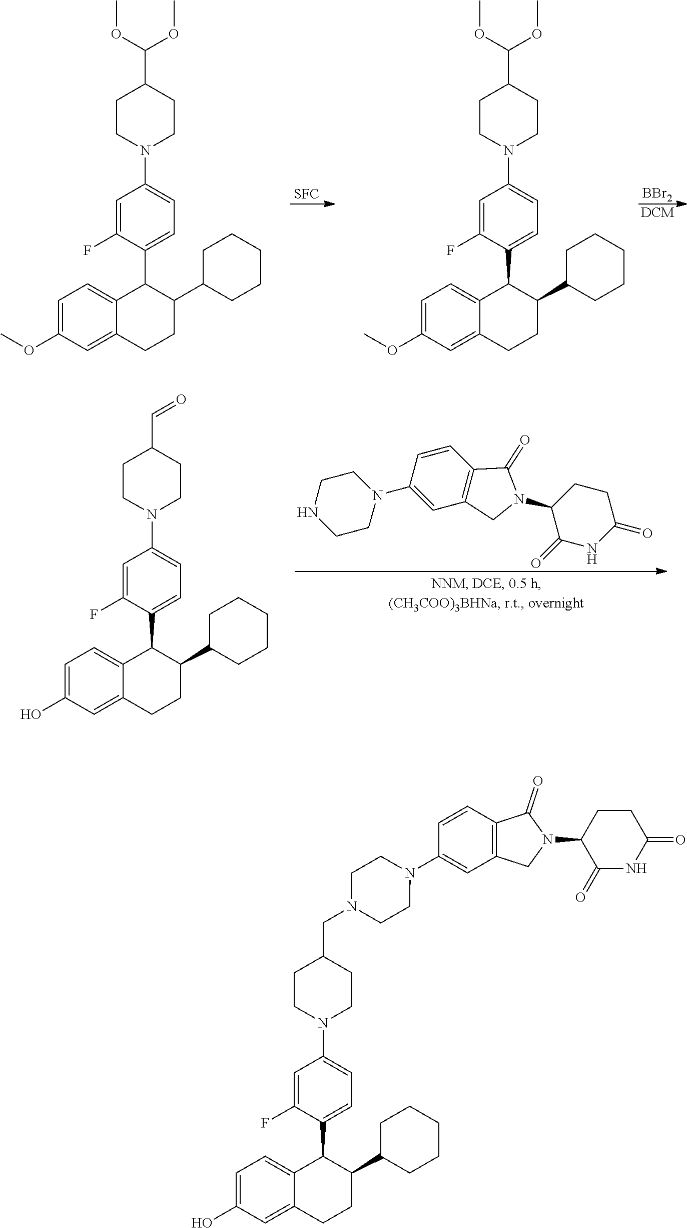

Step 1: 1-(4-(2-cyclohexyl-6-methoxy-1,2,3,4-tetrahydronaphthalen-1-yl)-3-fluorophenyl)-4-(dimethoxymethyl)piperidine (200 mg, 1 eq., 403 μmol) was purified by SFC (Column: DAICEL CHIRALPAK AD (250 mm*30 mm, 10 um); Mobile phase: 0.1% NH₃H₂O EtOH; from 25% to 25%; Flow rate: 70 mL/min) to give 1-(4-((1S,2R)-2-cyclohexyl-6-methoxy-1,2,3,4-tetrahydronaphthalen-1-yl)-3-fluorophenyl)-4-(dimethoxymethyl)piperidine (60 mg, 0.12 mmol, 30%) as a white solid. LC-MS (ESI⁺) m/z: 496.3 (M+H)⁺.

Step 2: To a solution of 1-(4-((1S,2R)-2-cyclohexyl-6-methoxy-1,2,3,4-tetrahydronaphthalen-1-yl)-3-fluorophenyl)-4-(dimethoxymethyl)piperidine (60 mg, 1 eq., 0.12 mmol) in DCM (3 mL) was added BBr₃ (0.15 g, 5 eq., 0.61 mmol). The mixture was stirred at 20° C. for 0.5 hour and then water (0.5 mL) in DCM (3 mL) was added dropwise at 20° C. The resulting mixture was stirred at 20° C. for 2 hour. LCMS showed the reaction was completed. The mixture was concentrated and then water (10 mL) was added. The mixture was extracted with DCM (10 mL×3). The combined organic layers were washed with brine (10 mL), dried over Na₂SO₄, filtered and concentrated. The mixture was concentrated in vacuum to give crude product. No further purification as it was used for the next step directly.

Step 3: A mixture of 1-(4-((1S,2R)-2-cyclohexyl-6-hydroxy-1,2,3,4-tetrahydronaphthalen-1-yl)-3-fluorophenyl)piperidine-4-carbaldehyde (60 mg, 137.75 μmol, 1 eq.), (S)-3-(1-oxo-5-(piperazin-1-yl)isoindolin-2-yl)piperidine-2,6-dione (54.28 mg, 165.30 μmol, 1.2 eq.) and NMM (13.93 mg, 137.75 μmol, 15.14 μL, 1 eq.) were added in DCE (3 mL). After addition, the mixture was stirred at 25° C. for 30 min, and then NaBH(OAc)₃ (72.99 mg, 344.37 μmol, 2.5 eq.) was added in the mixture. The reaction mixture was stirred at 25° C. for 16 h. LCMS showed the reaction was completed. The residue was purified by preparative HPLC(Column: Boston Green ODS 150 $30 mm+5 um; Condition water(FA)-CAN, B %: 25%-55%, 12 min) followed by lyophilization to yield (S)-3-(5-(4-((1-(4-((1S,2R)-2-cyclohexyl-6-hydroxy-1,2,3,4-tetrahydronaphthalen-1-yl)-3-fluorophenyl)piperidin-4-yl)methyl)piperazin-1-yl)-1-oxoisoindolin-2-yl)piperidine-2,6-dione (25 mg, 32.09 μmol, 23.29% yield, 96% purity) as a white solid. LC-MS (ESI+) m/z: 748.5 (M+H)⁺.

LCMS: calc. for C₄₅H₅₄FN₅O₄: 747.42, found: [M+H]⁺ 748.5.

HPLC: 95.5% purity at 220 nm.

¹H NMR: ¹H NMR (400 MHZ, METHANOL-d₄) δ ppm 8.28-8.45 (1H, m), 7.65 (1H, d, J=8.34 Hz), 7.07-7.12 (2H, m), 6.63-6.67 (1H, m), 6.58-6.62 (3H, m), 6.54 (1H, d, J=2.15 Hz), 6.45 (1H, dd, J=8.34, 2.38 Hz), 5.10 (1H, dd, J=13.29, 5.19 Hz), 4.46 (1H, d, J=4.53 Hz), 4.40 (2H, d, J=5.84 Hz), 3.66-3.74 (2H, m), 3.40 (4H, br d, J=4.65 Hz), 2.86-2.95 (2H, m), 2.78 (2H, br s), 2.71-2.76 (4H, m), 2.69 (1H, br s), 2.56-2.66 (1H, m), 2.41-2.52 (3H, m), 2.10-2.18 (1H, m), 2.02-2.07 (1H, m), 1.89 (2H, br d, J=13.47 Hz), 1.76-1.82 (2H, m), 1.72 (2H, br d, J=13.23 Hz), 1.56-1.65 (3H, m), 1.32-1.40 (2H, m), 1.25 (2H, br d, J=12.52 Hz), 1.19 (2H, br d, J=1.79 Hz), 1.02-1.10 (2H, m), 0.80 (1H, br d, J=11.32 Hz).

### Example 205. Preparation of (I-136) (S)-3-(5-(4-((1-(4-((1R,2S)-2-cyclohexyl-6-hydroxy-1,2,3,4-tetrahydronaphthalen-1-yl)-3-fluorophenyl)piperidin-4-yl)methyl)piperazin-1-yl)-1-oxoisoindolin-2-yl)piperidine-2,6-dione

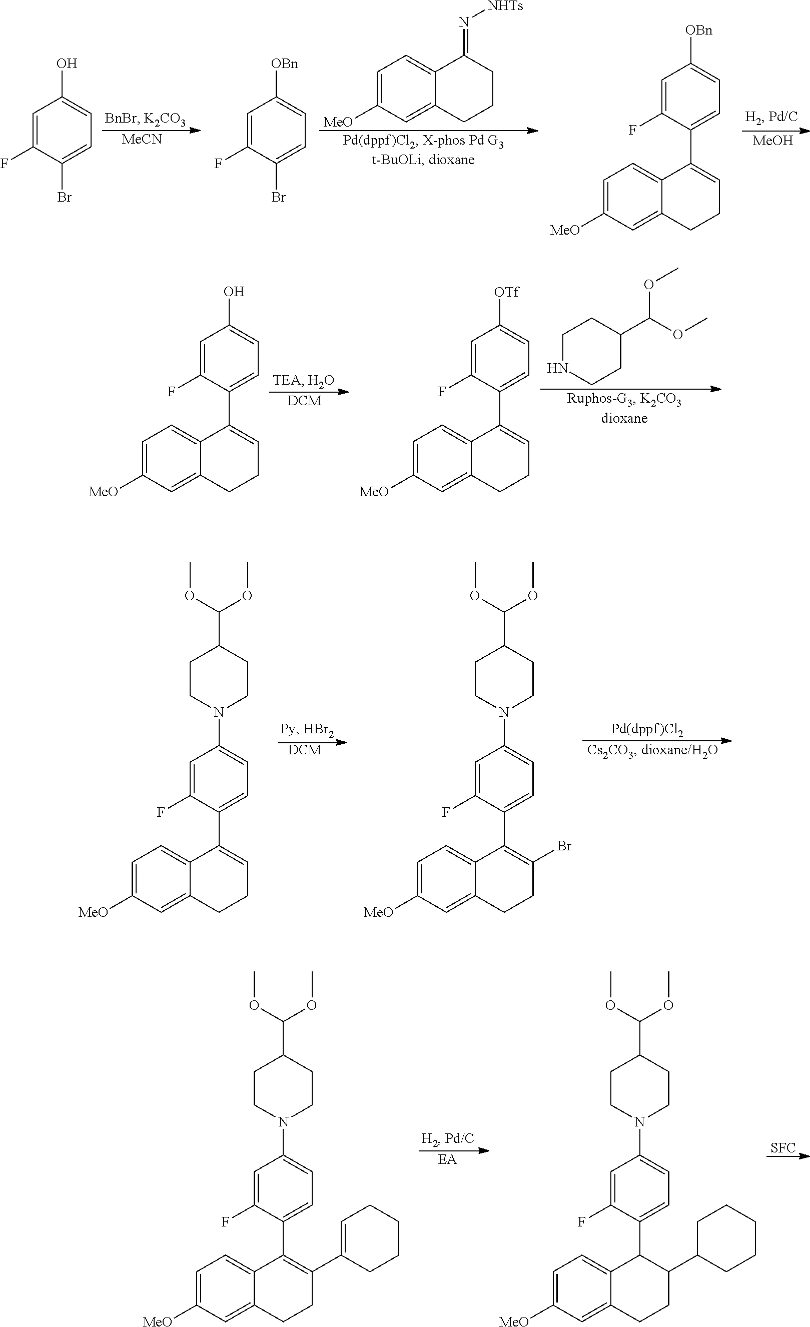

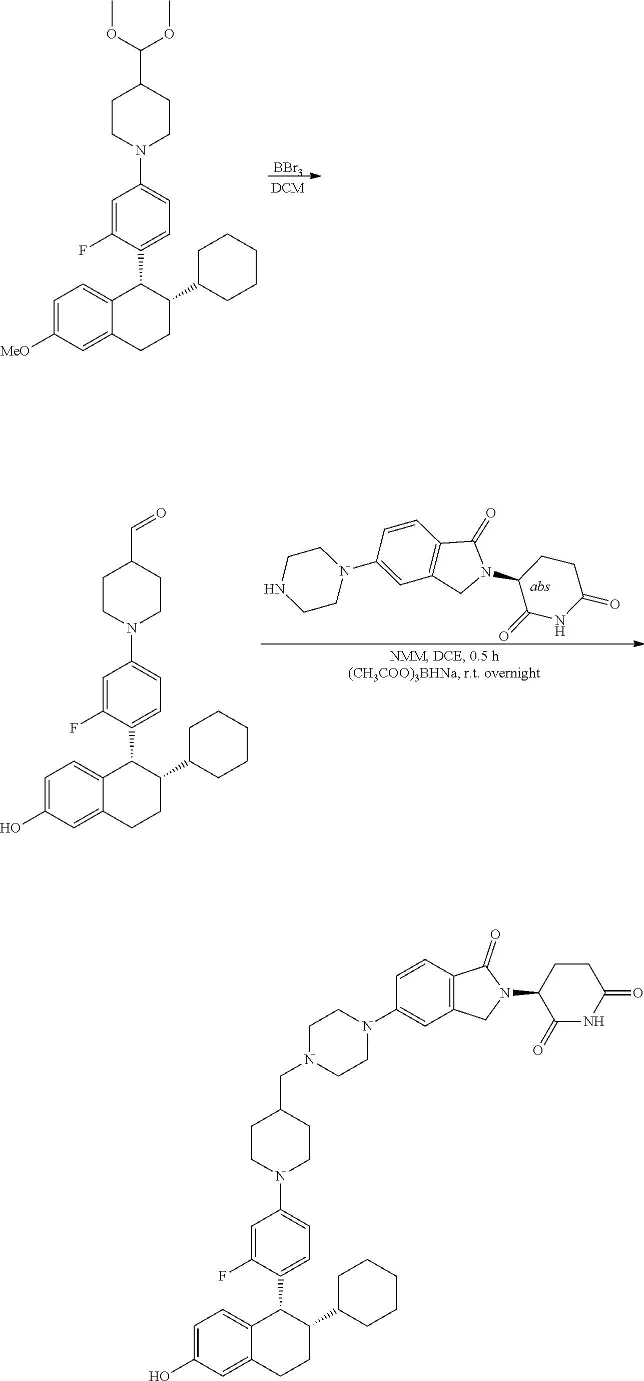 [SMILES: COc1ccc2c(c1)CC[C@@H](C1CCCCC1)[C@H]2c1ccc(N2CCC(C(OC)OC)CC2)cc1F ; "BBr3" ; O=CC1CCN(c2ccc([C@H]3c4ccc(O)cc4CC[C@H]3C3CCCCC3)c(F)c2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7C7CCCCC7)c(F)c6)CC5)CC4)ccc3C2=O)C(=O)N1]

Step 1: To a solution of 4-bromo-3-fluorophenol (2.00 g, 1 eq., 10.5 mmol) and (bromomethyl)benzene (1.97 g, 1.1 eq., 11.5 mmol) in MeCN (30 mL) was added K₂CO₃ (3.62 g, 2.5 eq., 26.2 mmol). The mixture was stirred at 70° C. for 3 hour. TLC showed the reaction was completed. The crude material was purified on silica gel column chromatography (from pure PE to PE/EtOAc=I/O) to give 4-(benzyloxy)-1-bromo-2-fluorobenzene (2.3 g, 8.2 mmol, 78%) as transparent oil.

¹H NMR (400 MHZ, DMSO-d₆) δ: ppm: 7.55 (t, J=8.58 Hz, 1H), 7.24-7.50 (m, 5H), 7.11 (dd, J=11.03, 2.44 Hz, 1H), 6.85 (dd, J=8.82, 2.15 Hz, 1H), 5.12 (s, 2H).

Step 2: A mixture of 4-(benzyloxy)-1-bromo-2-fluorobenzene (2.30 g, 1 eq., 8.18 mmol), (E)-N′-(6-methoxy-3,4-dihydronaphthalen-1 (2H)-ylidene)-4-methylbenzenesulfonohydrazide (3.10 g, 1.1 eq., 9.00 mmol), XPhos Pd G3 (346 mg, 0.05 eq., 409 μmol), Lithium 2-methyl-2-propanolate (1.96 g, 2.28 mL, 3 eq., 24.5 mmol) and PdCl₂(dppf) (599 mg, 0.1 eq., 818 μmol) in Dioxane (100 mL) was degassed and purged with N₂ for 3 times, and then the mixture was stirred at 100° C. for 12 hour under N₂ atmosphere. LCMS showed the reaction was completed. The reaction mixture was quenched by addition EA 30 mL at 20° C., and then diluted with water 50 mL and extracted with EA 90 mL (30 mL×3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=20/1) to give 4-(4-(benzyloxy)-2-fluorophenyl)-7-methoxy-1,2-dihydronaphthalene (2.8 g, 7.8 mmol, 95%) as a yellow solid.

LC-MS (ESI+) m/z: 360.9 (M+H⁺) ¹H NMR (400 MHZ, DMSO-d₆) δ: ppm 7.29-7.53 (m, 5H), 7.21 (t, J=8.58 Hz, 1H), 6.85-6.99 (m, 2H), 6.81 (d, J=2.38 Hz, 1H), 6.63-6.69 (m, 1H), 6.55-6.62 (m, 1H), 5.89 (t, J=4.59 Hz, 1H), 5.15 (s, 2H), 3.73 (s, 3H), 2.77 (t, J=7.99 Hz, 2H), 2.32 (td, J=7.87, 4.77 Hz, 2H).

Step 3: To a solution of 4-(4-(benzyloxy)-2-fluorophenyl)-7-methoxy-1,2-dihydronaphthalene (2.1 g, 1 eq., 5.8 mmol) in Ethyl acetate (30 mL) was added Pd/C (10%, 0.5 g) under N₂ atmosphere. The suspension was degassed and purged with H₂ for 3 times. The mixture was stirred under H₂ (15 psi.) at 20° C. for 12 hour. LCMS showed the reaction was completed. After filtration, the filtrate was concentrated. The crude material was purified on silica gel column chromatography (from pure PE to PE/EtOAc=2/1) to give 3-fluoro-4-(6-methoxy-3,4-dihydronaphthalen-1-yl)phenol (1.0 g, 3.7 mmol, 63%) as a yellow solid.

LC-MS (ESI+) m/z: 271.2 (M+H⁺).

¹H NMR (400 MHZ, DMSO-d₆) δ: ppm 7.47-7.52 (m, 2H), 7.40-7.46 (m, 2H), 7.35-7.40 (m, 1H), 6.95 (d, J=9.42 Hz, 2H), 6.84 (d, J=2.62 Hz, 1H), 6.67 (dd, J=8.52, 2.68 Hz, 1H), 6.48 (d, J=8.58 Hz, 1H), 5.17 (s, 2H), 3.73 (s, 3H), 2.96-3.02 (m, 2H), 2.88-2.95 (m, 2H).

Step 4: To a solution of 3-fluoro-4-(6-methoxy-3,4-dihydronaphthalen-1-yl)phenol (1.00 g, 1 eq., 3.70 mmol) and TEA (1.12 g, 1.55 mL, 3 eq., 11.1 mmol) in DCM (20 mL) was added Tf2O (1.25 g, 750 μL, 1.2 eq., 4.44 mmol). The mixture was stirred at 0° C. for 1 hour. LCMS showed the reaction was completed. The reaction mixture was quenched by addition water 10 mL at 20° C., and then diluted with water 10 mL and extracted with DCM 30 mL (10 mL×3). The combined organic layers were washed with DCM 15 mL (5 mL×3), dried over Na₂SO₄, filtered and concentrated under reduced pressure to give a residue. The crude material was purified on silica gel column chromatography (from PE/EtOAc=I/O to 20/1) to yield 3-fluoro-4-(6-methoxy-3,4-dihydronaphthalen-1-yl)phenyl trifluoromethanesulfonate (0.80 g, 2.0 mmol, 54%) as a colourless oil.

LC-MS (ESI+) m/z: 403.6 (M+H⁺)

¹H NMR (500 MHz, DMSO-d₆) δ: ppm 7.66 (dd, J=9.84, 2.37 Hz, 1H), 7.49-7.55 (m, 1H), 7.44 (dd, J=8.54, 2.29 Hz, 1H), 7.27 (dd, J=8.54, 2.14 Hz, 1H), 7.04 (t, J=8.62 Hz, 1H), 6.84 (d, J=2.44 Hz, 1H), 6.68 (dd, J=8.47, 2.67 Hz, 1H), 6.53 (dd, J=8.39, 1.22 Hz, 1H), 6.02 (t, J=4.50 Hz, 1H), 3.73 (s, 3H), 2.80 (t, J=8.09 Hz, 2H), 2.37 (td, J=7.82, 4.81 Hz, 2H)

Step 5: A mixture of 3-fluoro-4-(6-methoxy-3,4-dihydronaphthalen-1-yl)phenyl trifluoromethanesulfonate (800 mg, 1 eq., 1.99 mmol), 4-(dimethoxymethyl)piperidine (348 mg, 1.1 eq., 2.19 mmol), (2-Dicyclohexylphosphino-2′,6′-diisopropoxy-1, l′-biphenyl) [2-(2′-amino-1,1′-biphenyl)]palladium(II)methanesulfonate (166 mg, 0.1 eq., 199 μmol) and K₂CO₃ (824 mg, 3 eq., 5.96 mmol) in Dioxane (20 mL) was degassed and purged with N₂ for 3 times, and then the mixture was stirred at 100° C. for 12 hour under N₂ atmosphere. LCMS showed the reaction was completed. The reaction mixture was quenched by addition EA 10 mL at 20° C., and then diluted with water 30 mL and extracted with EA 30 mL (10 mL×3). The combined organic layers were washed with EA 30 mL (10 mL*3), dried over Na₂SO₄, filtered and concentrated under reduced pressure to give a residue. The crude material was purified on silica gel column chromatography (from pure PE to PE/EA=9/1) to as give 4-(dimethoxymethyl)-1-(3-fluoro-4-(6-methoxy-3,4-dihydronaphthalen-1-yl)phenyl)piperidine (700 mg, 1.3 mmol, 64%, 75% Purity) a yellow oil. LC-MS (ESI+) m/z: 412.1 (M+H⁺).

Step 6: To a solution of 4-(dimethoxymethyl)-1-(3-fluoro-4-(6-methoxy-3,4-dihydronaphthalen-1-yl)phenyl)piperidine (800 mg, 1.0 eq., 1.94 mol) in DCM (20 mL) was added Pyridinium bromide perbromide (373 mg, 0.6 eq., 1.17 mol). The mixture was stirred at 20° C. for 1 hour. LCMS showed the reaction was completed. The reaction mixture was quenched by addition water 30 mL at 20° C., and then diluted with extracted with EA 60 mL (20 mL×3). The combined organic layers were washed with EA 60 mL (20 mL×3), dried over Na₂SO₄, filtered and concentrated under reduced pressure to give a residue. The crude material was purified on silica gel column chromatography (from pure PE to PE/EtOAc=5/1) to give 1-(4-(2-bromo-6-methoxy-3,4-dihydronaphthalen-1-yl)-3-fluorophenyl)-4-(dimethoxymethyl)piperidine (550 mg, 1.12 mmol, 57.7%, 70% Purity) as a yellow solid.

LC-MS (ESI+) m/z: 490.1 (M+H⁺).

Step 7: A mixture of 1-(4-(2-bromo-6-methoxy-3,4-dihydronaphthalen-1-yl)-3-fluorophenyl)-4-(dimethoxymethyl)piperidine (550 mg, 1 eq., 1.12 mmol), cyclohex-1-en-1-ylboronic acid (155 mg, 1.1 eq., 1.23 mmol), Cs₂CO₃ (1.10 g, 3 eq., 3.36 mmol) and PdCl₂(dppf) (82.1 mg, 0.1 eq., 112 μmol) in water (2 mL) and Dioxane (10 mL) was degassed and purged with N₂ for 3 times, and then the mixture was stirred at 80° C. for 12 hour under N₂ atmosphere. LCMS showed the reaction was completed. The reaction mixture was quenched by addition EA 30 mL at 20° C., and then diluted with water 30 mL and extracted with EA 90 mL (30 mL×3). The combined organic layers were washed with EA 30 mL (10 mL×3), dried over Na₂SO₄, filtered and concentrated under reduced pressure to give a residue. The crude material was purified on silica gel column chromatography (from pure PE to PE/EtOAc=3/1) to give 1-(4-(2-(cyclohex-1-en-1-yl)-6-methoxy-3,4-dihydronaphthalen-1-yl)-3-fluorophenyl)-4-(dimethoxymethyl)piperidine (450 mg, 0.67 mmol, 60%, 73% Purity) as a yellow solid. LC-MS (ESI+) m/z; 492.2 (M+H⁺).

Step 8: To a solution of 1-(4-(2-(cyclohex-1-en-1-yl)-6-methoxy-3,4-dihydronaphthalen-1-yl)-3-fluorophenyl)-4-(dimethoxymethyl)piperidine (400 mg, 1 eq., 814 μmol) in Ethyl acetate (15 mL) was added Pd/C (10%, 0.2 g) under N₂ atmosphere. The suspension was degassed and purged with H₂ for 3 times. The mixture was stirred under H₂ (50 Psi) at 70° C. for 72 hour. LCMS showed the reaction was completed. The crude was purified by prep-HPLC together (Welch Xtimate C18 150*25 mm*5 um, water(FA)-ACN as a mobile phase, from 90% to 100%, Gradient Time (min): 11, Flow Rate (ml/min): 25) to give 1-(4-(2-cyclohexyl-6-methoxy-1,2,3,4-tetrahydronaphthalen-1-yl)-3-fluorophenyl)-4-(dimethoxymethyl)piperidine (200 mg, 403 μmol, 49.6%). LC-MS (ESI+) m/z: 496.4 (M+H⁺).

Step 9: 1-(4-(2-cyclohexyl-6-methoxy-1,2,3,4-tetrahydronaphthalen-1-yl)-3-fluorophenyl)-4-(dimethoxymethyl)piperidine (200 mg, 1 eq., 403 μmol) was separated by SFC(Column: DAICEL CHIRALPAK AD (250 mm*30 mm, 10 um); Mobile phase: 0.1% NH₃H₂O ETOH; from 25% to 25%; Flow rate: 70 mL/min) to give 1-(4-((1R,2S)-2-cyclohexyl-6-methoxy-1,2,3,4-tetrahydronaphthalen-1-yl)-3-fluorophenyl)-4-(dimethoxymethyl)piperidine (60 mg, 0.12 mmol, 30%) as white solid. LC-MS (ESI+) m/z: 496.4 (M+H⁺).

Step 10: To a solution of 1-(4-(1R,2S)-2-cyclohexyl-6-methoxy-1,2,3,4-tetrahydronaphthalen-1-yl)-3-fluorophenyl)-4-(dimethoxymethyl)piperidine (60 mg, 1 eq., 0.12 mmol) in DCM (3 mL) was added BBr₃ (0.15 g, 57 μL, 5 eq., 0.61 mmol). The mixture was stirred at 20° C. for 1 hour and then water (0.5 mL) in DCM (3 mL) was added dropwise at 20° C., the resulting mixture was stirred at 20° C. for 2 hour. LCMS showed the reaction was completed. The mixture was concentrated and then water (10 mL) was added. The mixture was extracted with DCM (10 mL×3). The combined organic layers were washed with brine (10 mL), dried over Na₂SO₄, filtered and concentrated. The mixture was concentrated in vacuo to give crude product. No further purification as it is used for the next step directly. LC-MS (ESI+) m/z: 496.4 (M+H⁺).

Step 11: To a solution of 1-(4-((1R,2S)-2-cyclohexyl-6-hydroxy-1,2,3,4-tetrahydronaphthalen-1-yl)-3-fluorophenyl)piperidine-4-carbaldehyde (30 mg, 1 eq., 69 μmol) and (S)-3-(1-oxo-5-(piperazin-1-yl)isoindolin-2-yl)piperidine-2,6-dione (25 mg, 1.1 eq., 76 μmol) in DCE (3 mL) was added dropwise 4-methylmorfolin (7.0 mg, 7.6 μL, 1 eq., 69 μmol) to pH=7 at 0.5 hour. After addition, the mixture was stirred at this temperature for 20° C., and then Sodium triacetoxyborohydride (15 mg, 10 μL, 1 eq., 69 μmol) was added dropwise at 20° C. The resulting mixture was stirred at 20° C. for 2 hour. LCMS showed the reaction was completed. The crude was purified by prep-HPLC together (Boston Green ODS 150*30 mm*5 um, water (FA)-ACN as a mobile phase, from 20% to 30%, Gradient Time (min): 12, Flow Rate (ml/min): 25) to give (S)-3-(5-(4-((1-(4-((1R,2S)-2-cyclohexyl-6-hydroxy-1,2,3,4-tetrahydronaphthalen-1-yl)-3-fluorophenyl)piperidin-4-yl)methyl)piperazin-1-yl)-1-oxoisoindolin-2-yl)piperidine-2,6-dione (11.6 mg, 15.5 μmol, 23%) as a white solid.

LCMS: calc. for C₄₅H₅₄FN₅O₄: 747.72, found: [M+H]⁺ 748.5.

HPLC: 95.74% purity at 220 nm.

¹H NMR (400 MHZ, METHANOL-d₄) δ: ppm 8.11-8.31 (m, 1H), 7.63-7.72 (m, 1H), 7.08-7.23 (m, 2H), 6.62-6.69 (m, 2H), 6.61 (d, J=2.74 Hz, 2H), 6.54 (d, J=2.26 Hz, 1H), 6.42-6.48 (m, 1H), 5.11 (dd, J=13.29, 4.95 Hz, 1H), 4.38-4.50 (m, 3H), 3.68-3.74 (m, 2H), 3.46-3.66 (m, 4H), 3.17 (d, J=2.50 Hz, 4H), 2.89-2.96 (m, 2H) 2.87 (s, 2H), 2.69-2.81 (m, 4H), 2.41-2.52 (m, 1H), 2.12-2.19 (m, 1H), 2.02-2.07 (m, 1H), 1.92-2.01 (m, 1H), 1.89 (d, J=13.47 Hz, 2H), 1.79 (dd, J=12.22, 6.85 Hz, 1H), 1.67-1.74 (m, 2H), 1.56-1.66 (m, 3H), 1.38-1.48 (m, 2H), 1.23-1.30 (m, 2H), 1.13-1.22 (m, 2H), 1.01-1.09 (m, 2H), 0.75-0.88 (m, 1H).

### Example 206. Preparation of (I-110) (S)-3-(5-(4-((1-(4-((1S,2R)-6-hydroxy-2-(3-(trifluoromethoxy)phenyl)-1,2,3,4-tetrahydronaphthalen-1-yl)phenyl)piperidin-4-yl)methyl)piperazin-1-yl)-1-oxoisoindolin-2-yl)piperidine-2,6-dione

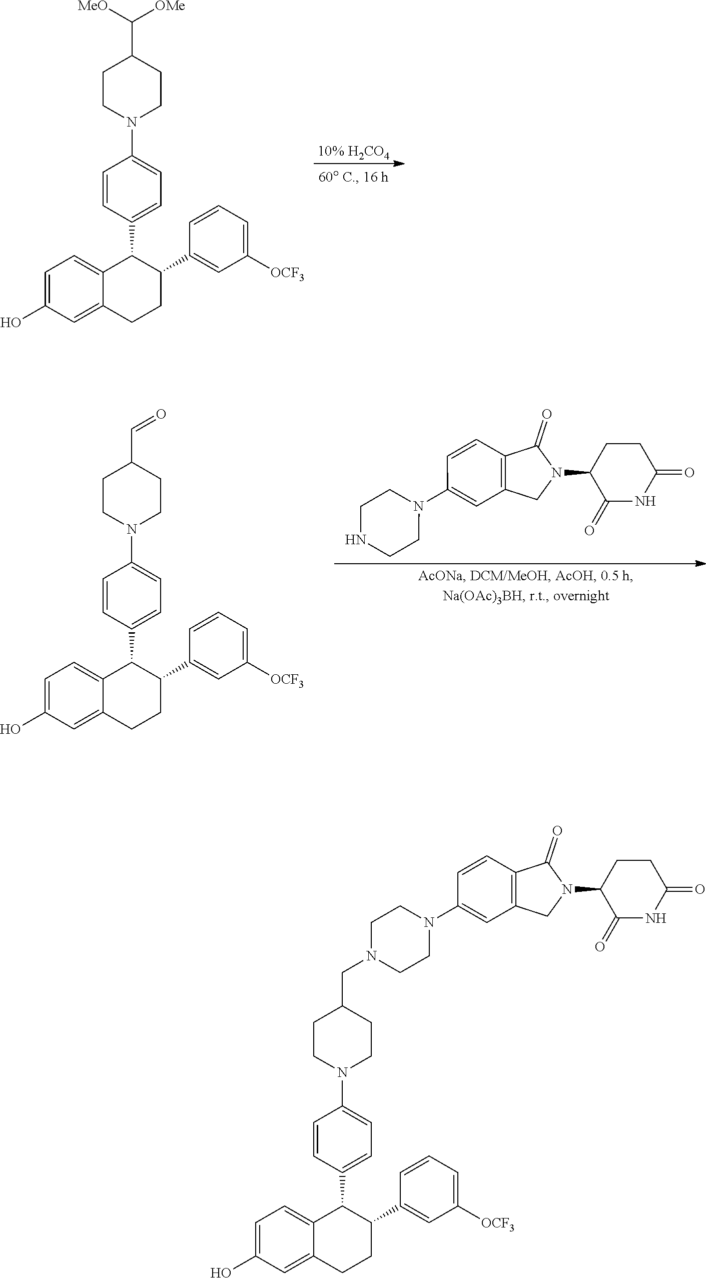 [SMILES: COC(OC)C1CCN(c2ccc([C@H]3c4ccc(O)cc4CC[C@H]3c3cccc(OC(F)(F)F)c3)cc2)CC1 ; "10% H2CO4" ; O=CC1CCN(c2ccc([C@H]3c4ccc(O)cc4CC[C@H]3c3cccc(OC(F)(F)F)c3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7c7cccc(OC(F)(F)F)c7)cc6)CC5)CC4)ccc3C2=O)C(=O)N1]

Step 1: To a solution of (1S,2R)-1-[4-[4-(dimethoxymethyl)-1-piperidyl]phenyl]-2-[3-(trifluoromethoxy)phenyl]tetralin-6-ol (130 mg, 240.03 μmol, 1 eq) in THF (4 mL) was added H₂SO₄ (4 mL, 10% purity). The mixture was stirred at 60° C. for 16 hr. LCMS showed desired MS was detected. The reaction was adjust to pH=7 with aq. NaHCO₃, then the residue was diluted with 40 mL H₂O and extracted with 100 mL EtOAc (50 mL*2). The combined organic layers dried over Na₂SO₄, filtered and concentrated under reduced pressure to give a residue. Compound 1-[4-[(1S,2R)-6-hydroxy-2-[3-(trifluoromethoxy)phenyl]tetralin-1-yl]phenyl]piperidine-4-carbaldehyde (85 mg, 171.53 μmol, 71.46% yield) was obtained as a yellow oil. LC-MS (ESI⁺) m/z: 514.3 (M+H₂O)⁺.

Step 2: To a solution of 1-(4-((1S,2R)-6-hydroxy-2-(3-(trifluoromethoxy)phenyl)-1,2,3,4-tetrahydronaphthalen-1-yl)phenyl)piperidine-4-carbaldehyde (85 mg, 171.53 μmol, 1 eq) and (3S)-3-(1-oxo-5-piperazin-1-yl-isoindolin-2-yl)piperidine-2,6-dione (85.86 mg, 171.53 μmol, 1 eq, p-TSA) in DCM (4 mL) and MeOH (4 mL) was added sodium acetate (42.21 mg, 514.60 μmol, 3 eq). The mixture was stirred at 25° C. for 0.5 hr. Then the mixture was added sodium triacetoxyboranuide (72.71 mg, 343.07 μmol, 2 eq) and acetic acid (30.90 mg, 514.60 μmol, 29.43 μL, 3 eq). The mixture was stirred at 25° C. overnight. LCMS showed desired MS was detected. The reaction mixture was diluted with H₂O 20 mL and extracted with DCM 100 mL (50 mL*2). The combined organic layers were washed with brine 40 mL (20 mL*2), dried over Na₂SO₄, filtered and concentrated under reduced pressure to give a residue. The residue was purified by prep.HPLC (column: Welch Xtimate C18 150*30 mm*5 um; mobile phase: [water(FA)-ACN]; B %: 28%-48%, 7 min). Compound C₄₆H₄₈O₅N₅F₃ (87 mg, 107.69 μmol, 62.78% yield) was obtained as a white solid. LC-MS (ESI+) m/z: 808.2 (M+H)⁺

HPLC: 100%, purity at 220 nm.

SFC: Rt: 2.347 min; Area, 87.568%;

¹H NMR (400 MHz, DMSO-d₆) δ ppm 10.91-11.00 (m, 1H) 9.06-9.23 (m, 1H) 8.18-8.30 (m, 0.400H) 7.52 (d, J=8.53 Hz, 1H) 7.27-7.33 (m, 1H) 7.03-7.13 (m, 3H) 6.95-7.01 (m, 1H) 6.62-6.68 (m, 2H) 6.59-6.62 (m, 1H) 6.53-6.57 (m, 2H) 6.47-6.51 (m, 1H) 6.19-6.25 (m, 2H) 5.01-5.09 (m, 1H) 4.29-4.42 (m, 1H) 4.12-4.24 (m, 2H) 3.52 (br d, J=7.53 Hz, 3H) 3.28 (br s, 6H) 2.86-3.01 (m, 3H) 2.30-2.44 (m, 4H) 2.16-2.23 (m, 2H) 2.11 (br d, J=6.27 Hz, 2H) 1.93-2.00 (m, 1H) 1.58-1.85 (m, 5H) 1.09-1.20 (m, 2H). Stereochemistry was arbitrarily assigned

### Example 207. Preparation of (I-109) (3S)-3-[5-[4-[[1-[4-[(1R,2S)-6-hydroxy-2-[3-(trifluoromethoxy)phenyl]tetralin-1-yl]phenyl]-4-piperidyl]methyl]piperazin-1-yl]-1-oxo-isoindolin-2-yl]piperidine-2,6-dione

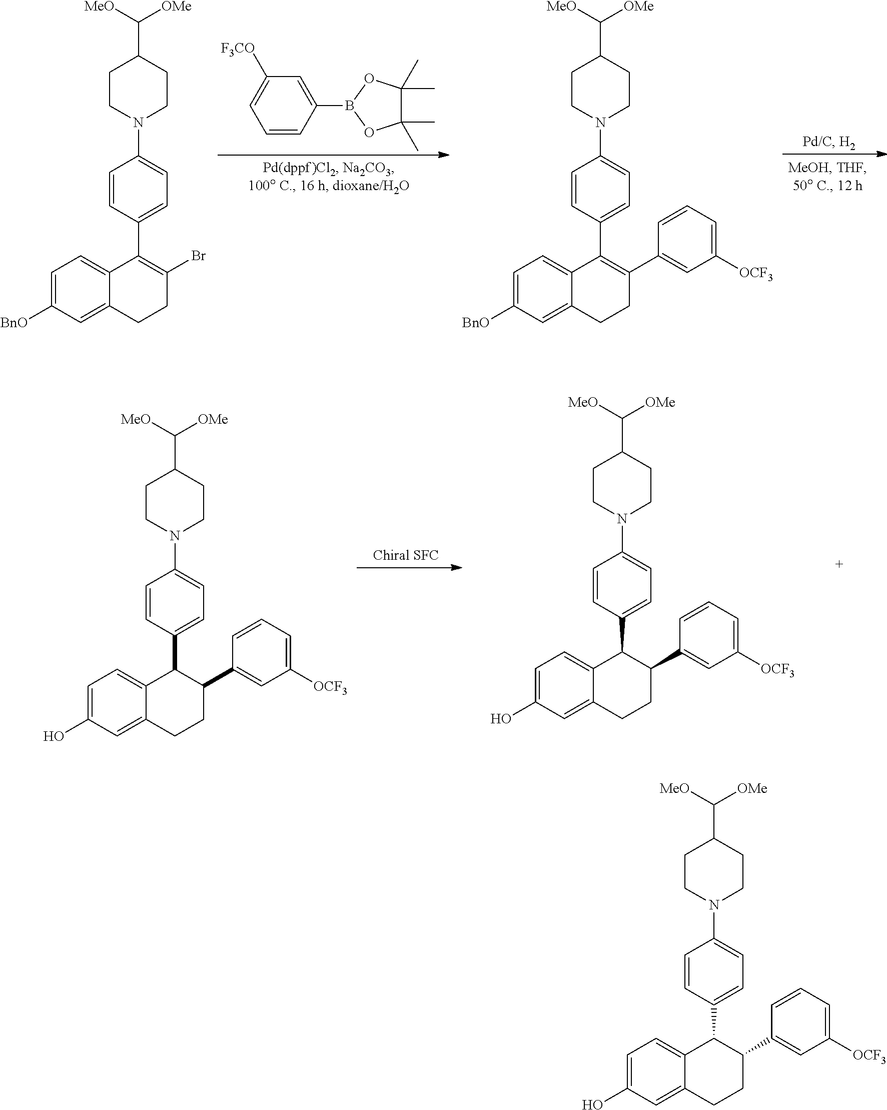 [SMILES: COC(OC)C1CCN(c2ccc(C3=C(Br)CCc4cc(OCc5ccccc5)ccc43)cc2)CC1 ; CC1(C)OB(c2cccc(OC(F)(F)F)c2)OC1(C)C ; COC(OC)C1CCN(c2ccc(C3=C(c4cccc(OC(F)(F)F)c4)CCc4cc(OCc5ccccc5)ccc43)cc2)CC1 ; COC(OC)C1CCN(c2ccc([C@@H]3c4ccc(O)cc4CC[C@@H]3c3cccc(OC(F)(F)F)c3)cc2)CC1 ; COC(OC)C1CCN(c2ccc([C@@H]3c4ccc(O)cc4CC[C@@H]3c3cccc(OC(F)(F)F)c3)cc2)CC1 ; COC(OC)C1CCN(c2ccc([C@H]3c4ccc(O)cc4CC[C@H]3c3cccc(OC(F)(F)F)c3)cc2)CC1]

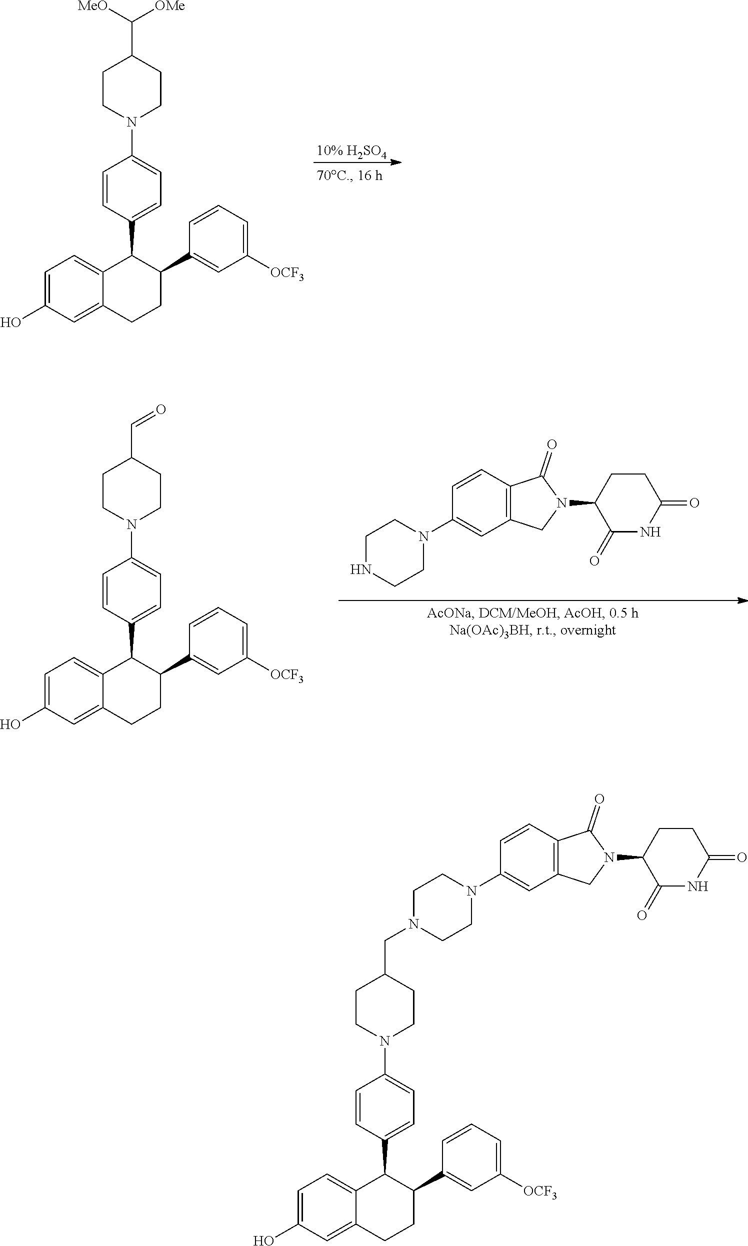 [SMILES: COC(OC)C1CCN(c2ccc([C@@H]3c4ccc(O)cc4CC[C@@H]3c3cccc(OC(F)(F)F)c3)cc2)CC1 ; O=CC1CCN(c2ccc([C@@H]3c4ccc(O)cc4CC[C@@H]3c3cccc(OC(F)(F)F)c3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc(OC(F)(F)F)c7)cc6)CC5)CC4)ccc3C2=O)C(=O)N1]

Step 1: A mixture of sodium carbonate (232 mg, 3 Eq, 2.19 mmol), Pd(dppf)Cl₂ (59.6 mg, 0.1 Eq, 72.9 μmol), 4,4,5,5-tetramethyl-2-(3-(trifluoromethoxy)phenyl)-1,3,2-dioxaborolane (252 mg, 1.2 Eq, 875 μmol), 1-(4-(6-(benzyloxy)-2-bromo-3,4-dihydronaphthalen-1-yl)phenyl)-4-(dimethoxymethyl)piperidine (400 mg, 1 Eq, 729 μmol) in 1,4-Dioxane (8 mL) and H₂O (2 mL) was degassed and purged with N₂ for 3 times, and then the mixture was stirred at 100° C. for 16 hr under N₂ atmosphere. LCMS showed 54.862% desired MS. The reaction was quenched with water (50 mL) and extracted with ethyl acetate (100 mL*2). The organic layer was washed with brine (20 mL) and dried over Na₂SO₄ and concentrated in vacuo to give a white solid. The white solid was subjected to column chromatography over silica gel (gradient elution: 0-100% EtOAc). The desired fractions were collected, and concentrated to dryness in vacuo to give 1-(4-(6-(benzyloxy)-2-(3-(trifluoromethoxy)phenyl)-3,4-dihydronaphthalen-1-yl)phenyl)-4-(dimethoxymethyl)piperidine (398 mg, 632.04 μmol, 99.5% yield) as a white solid. LC-MS (ESI⁺) m/z: 605.0 (M+H)⁺.

Step 2: A mixture of 1-[4-[6-benzyloxy-2-[3-(trifluoromethoxy)phenyl]-3,4-dihydronaphthalen-1-yl]phenyl]-4-(dimethoxymethyl)piperidine (400 mg, 635.22 μmol, 1 eq), Pd/C (540.80 mg, 508.17 μmol, 10% purity, 0.8 eq) in THF (5 mL) and MeOH (5 mL) was degassed and purged with H₂ for 3 times, and then the mixture was stirred at 50° C. for 12 br under H₂ atmosphere. The suspension was degassed and purged with dihydrogen (1.08 mg, 1 Eq, 534 μmol) for 10 mins. The mixture was stirred under 15 psi H₂ (1.08 mg, 1 Eq, 534 μmol) at 50° C. for 12 hr. LCMS showed 84.236% desired MS. The reaction was filtrate and concentrated. The residue was further separated by prep-HPLC (TFA condition; or neutral condition; or basic condition) to give desired 1-[4-[4-(dimethoxymethyl)-1-piperidyl]phenyl]-2-[3-(trifluoromethoxy)phenyl]tetralin-6-ol (400 mg, 508.17 μmol, 80% yield, purity 95%) as a white oil. LC-MS (ESI⁺) m/z: 542.1 (M+H)⁺.

Step 3: 540.8 mg of 1-[4-[4-(dimethoxymethyl)-1-piperidyl]phenyl]-2-[3-(trifluoromethoxy)phenyl]tetralin-6-ol was further separated by SFC (column: DAICEL CHIRALCEL ODH (250 mm*30 mm, 5 um); mobile phase: [0.1% NH3H2O ETOH]; B %: 35%-35%, 45 min) to give desired (1R,2S)-1-[4-[4-(dimethoxymethyl)-1-piperidyl]phenyl]-2-[3-(trifluoromethoxy)phenyl]tetralin-6-ol (70 mg, 129.25 mmol, 20.35% yield) as a white oil and desired (1S,2R)-1-[4-[4-(dimethoxymethyl)-1-piperidyl]phenyl]-2-[3-(trifluoromethoxy)phenyl]tetralin-6-ol (120 mg, 221.57 mmol, 34.88% yield) as a white oil.

LC-MS (ESI⁺) m/z: 542.1 (M+H)⁺.

Step 4: To a solution of (1R,2S)-1-[4-[4-(dimethoxymethyl)-1-piperidyl]phenyl]-2-[3-(trifluoromethoxy)phenyl]tetralin-6-ol (112.00 mg, 206.79 μmol, 1 eq) in THF (4 mL) was added H₂SO₄ (4 mL, 10% purity). The mixture was stirred at 70° C. for 16 hr. LCMS showed desired MS was detected. The reaction was adjust to pH=7 with NaHCO₃, Then the residue was diluted with 40 mL H₂O and extracted with EtOAc 100 mL (50 mL*2). The combined organic layers dried over Na₂SO₄, filtered and concentrated under reduced pressure to give a residue. Compound 1-[4-[(1R,2S)-6-hydroxy-2-[3-(trifluoromethoxy)phenyl]tetralin-1-yl]phenyl]piperidine-4-carbaldehyde (95 mg, 191.71 μmol, 92.71% yield) was obtained as a yellow oil. LC-MS (ESI⁺) m/z: 514.3 (M+H₂O)⁺.

Step 5: To a solution of 1-[4-[(1R,2S)-6-hydroxy-2-[3-(trifluoromethoxy)phenyl]tetralin-1-yl]phenyl]piperidine-4-carbaldehyde (95.00 mg, 191.71 μmol, 1 eq) and (38)-3-(1-oxo-5-piperazin-1-yl-isoindolin-2-yl)piperidine-2,6-dione (95.97 mg, 191.71 μmol, 1 eq, p-TSA) in DCM (4 mL) and MeOH (4 mL) was added sodium acetate (47.18 mg, 575.14 μmol, 3 eq). The mixture was stirred at 25° C. for 0.5 hr. Then the mixture was added sodium triacetoxyboranuide (81.26 mg, 383.43 μmol, 2 eq) and acetic acid (158.81 mg, 575.14 μmol, 151.25 μL, 3 eq). The mixture was stirred at 25° C. overnight. LCMS showed desired MS was detected. The reaction mixture was diluted with 20 mL H₂O and extracted with DCM (50 mL*2). The combined organic layers were washed with brine (20 mL*2), dried over Na₂SO₄, filtered and concentrated under reduced pressure to give a residue. The residue was purified by prep.HPLC (column: Welch Xtimate C18 150*30 mm*5 um; mobile phase: [water(FA)-ACN]; B %: 28%-48%, 7 min). Compound (3S)-3-[5-[4-[[1-[4-[(1R,2S)-6-hydroxy-2-[3-(trifluoromethoxy)phenyl]tetralin-1-yl]phenyl]-4-piperidyl]methyl]piperazin-1-yl]-1-oxo-isoindolin-2-yl]piperidine-2,6-dione (92 mg, 113.88 μmol, 59.40% yield) was obtained as a white solid.

LCMS (ESI+) m/z: 808.2 (M+H)⁺

HPLC: 100%, purity at 220 nm.

SFC: Rt: 2.321 min; Area, 82.355%; method:
Ic_MeOH_DEA_MeCN_50_1ML_10_MIN_10_CM.

¹H NMR (400 MHZ, DMSO-d₆) δ ppm 10.92-10.99 (m, 1H) 9.06-9.21 (m, 1H) 8.20-8.26 (m, 1H) 7.45-7.56 (m, 1H) 7.23-7.35 (m, 1H) 7.03-7.16 (m, 3H) 6.93-7.01 (m, 1H) 6.63-6.71 (m, 2H) 6.59-6.61 (m, 1H) 6.53-6.57 (m, 2H) 6.47-6.51 (m, 1H) 6.15-6.27 (m, 2H) 4.98-5.11 (m, 1H) 4.27-4.38 (m, 1H) 4.08-4.25 (m, 2H) 3.47-3.57 (m, 4H) 3.29 (br s, 5H) 2.83-3.06 (m, 4H) 2.31-2.42 (m, 3H) 2.13-2.23 (m, 3H) 1.89-2.08 (m, 2H) 1.59-1.80 (m, 5H) 1.07-1.23 (m, 2H). Stereochemistry was arbitrarily assigned

### Example 208. Preparation of (I-130) (35)-3-[5-[4-[[1-[4-[(1S, 2R)-6-hydroxy-2-(4-hydroxyphenyl)tetralin-1-yl]phenyl]-4-piperidyl]methyl]piperazin-1-yl]-1-oxo-isoindolin-2-yl]piperidine-2,6-dione and (I-131) (35)-3-[5-[4-[[1-[4-[(1R, 2S)-6-hydroxy-2-(4-hydroxyphenyl)tetralin-1-yl]phenyl]-4-piperidyl]methyl]piperazin-1-yl]-1-oxo-isoindolin-2-yl]piperidine-2,6-dione

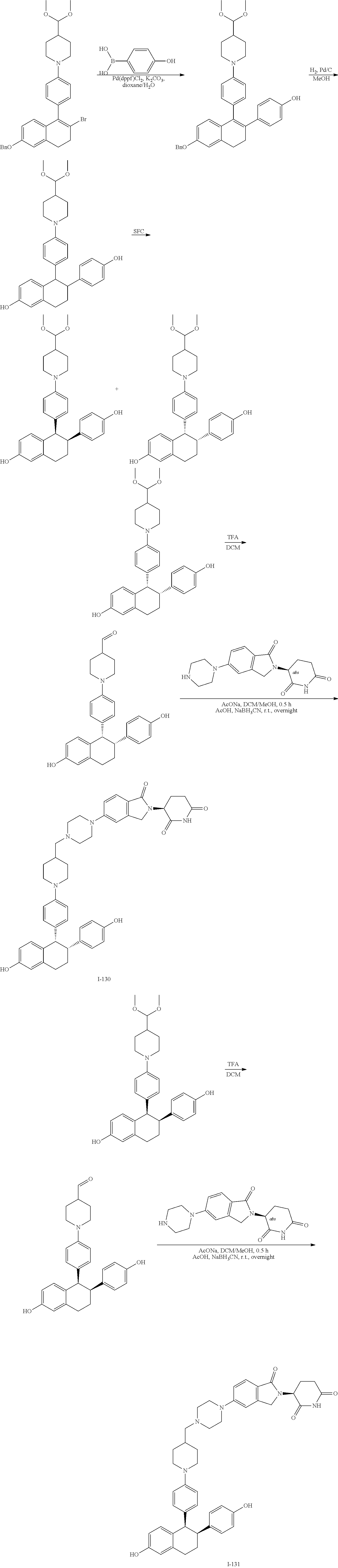

Step 1: To a solution of 1-[4-(6-benzyloxy-2-bromo-3,4-dihydronaphthalen-1-yl)phenyl]-4-(dimethoxymethyl)piperidine (1 g, 1.8 mmol, 1 eq) and (4-hydroxyphenyl)boronic acid (377.19 mg, 2.73 mmol, 1.5 eq) in dioxane (10 mL) and H₂O (2.5 mL) was added Pd(dppf)Cl₂ (133.40 mg, 182.31 μmol, 0.1 eq) and Na₂CO₃ (386.46 mg, 3.65 mmol, 2 eq). The mixture was stirred at 100° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue. The residue was purified by flash silica gel chromatography (from pure PE to PE/EtOAc=3/1, TLC: PE/EtOAc=3/1, R_{f}=0.21) to yield 4-[6-benzyloxy-1-[4-[4-(dimethoxymethyl)-1-piperidyl]phenyl]-3,4-dihydronaphthalen-2-yl]phenol (960 mg, 1.67 mmol, 91.87% yield, 98% purity) as a brown solid. LC-MS (ESI⁺) m/z 562.4 [M+H]⁺.

¹H NMR (500 MHz, CDCl₃)₂=7.46-7.42 (m, 2H), 7.41-7.36 (m, 2H), 7.35-7.30 (m, 1H), 6.95-6.87 (m, 4H), 6.86-6.78 (m, 3H), 6.75 (d, J=8.5 Hz, 1H), 6.65 (dd, J=2.6, 8.5 Hz, 1H), 6.57 (d, J=8.7 Hz, 2H), 5.06 (s, 2H), 4.11-4.09 (m, 1H), 3.70 (br d, J=12.2 Hz, 2H), 3.39 (s, 5H), 3.44-3.35 (m, 1H), 2.92 (brt, J=7.7 Hz, 2H), 2.78-2.71 (m, 2H), 2.65 (t, J=11.7 Hz, 2H), 1.87 (br d, J=12.7 Hz, 2H), 1.80-1.71 (m, 1H), 1.53-1.42 (m, 2H).

Step 2: To a solution of 4-[6-benzyloxy-1-[4-[4-(dimethoxymethyl)-1-piperidyl]phenyl]-3,4-dihydronaphthalen-2-yl]phenol (800 mg, 1.40 mmol, 98% purity, 1 eq) in MeOH (10 mL) was added Pd/C (800 mg, 10% purity). The mixture was stirred at 25° C. for 5 h. The reaction mixture was filtered and concentrated to yield a residue. The residue was separated by chiral SFC (column: DAICEL CHIRALCEL OD-H (250 mm*30 mm, 5 um); mobile phase: [0.1% NH₃H₂O MEOH]; B %: 45%-45%, 45 min), followed by lyophilization to yield (1S,2R)-1-[4-[4-(dimethoxymethyl)-1-piperidyl]phenyl]-2-(4-hydroxyphenyl)tetralin-6-ol (240 mg, 486.48 μmol, 44.14% yield, 96% purity) as a white solid and (1R,2S)-1-[4-[4-(dimethoxymethyl)-1-piperidyl]phenyl]-2-(4-hydroxyphenyl)tetralin-6-ol (290 mg, 606.21 μmol, 55.0% yield, 99% purity) as a white solid. LC-MS (ESI⁺) m/z 474.3 [M+H]⁺. ¹H NMR of (1S,2R)-1-[4-[4-(dimethoxymethyl)-1-piperidyl]phenyl]-2-(4-hydroxyphenyl)tetralin-6-ol: (400 MHz, CDCl₃)₂=6.82 (d, J=8.3 Hz, 1H), 6.71-6.54 (m, 8H), 6.33 (d, J=8.6 Hz, 2H), 4.16 (d, J=4.8 Hz, 1H), 4.10 (d, J=7.3 Hz, 1H), 3.59 (t, J=10.8 Hz, 2H), 3.38 (s, 5H), 3.39-3.37 (m, 1H), 3.34-3.24 (m, 1H), 3.09-2.97 (m, 2H), 2.66-2.52 (m, 2H), 2.20-2.04 (m, 1H), 1.84 (br d, J=13.1 Hz, 2H), 1.79-1.66 (m, 4H), 1.49 (br s, 2H) ¹H NMR of (1R,2S)-1-[4-[4-(dimethoxymethyl)-1-piperidyl]phenyl]-2-(4-hydroxyphenyl)tetralin-6-ol: (400 MHz, CDCl₃)₂=6.84 (d, J=8.3 Hz, 1H), 6.72-6.56 (m, 8H), 6.32 (d, J=8.7 Hz, 2H), 4.16 (d, J=4.6 Hz, 1H), 4.09 (d. J=7.4 Hz, 1H), 3.59 (t, J=10.1 Hz, 2H), 3.38 (s, 6H), 3.33-3.25 (m, 1H), 3.10-2.95 (m, 2H), 2.57 (t, J=12.2 Hz, 2H), 2.21-2.05 (m, 1H), 1.83 (br d, J=12.3 Hz, 2H), 1.79-1.62 (m, 4H), 1.51-1.37 (m, 2H)

Step 3: To a solution of (1S,2R)-1-[4-[4-(dimethoxymethyl)-1-piperidyl]phenyl]-2-(4-hydroxyphenyl)tetralin-6-ol (100 mg, 202.7 μmol, 96% purity, 1 eq) in DCM (2.5 mL) was added TFA (770.00 mg, 6.75 mmol, 0.5 mL, 33.32 eq). The mixture was stirred at 25° C. for 1 h. The reaction mixture was quenched by addition of water (30 mL), extracted with EtOAc (20 mL×3). The combined organic layers were washed with brine (10 mL), dried over Na₂SO₄, filtered and concentrated under reduced pressure to yield 1-[4-[(1S,2R)-6-hydroxy-2-(4-hydroxyphenyl)tetralin-1-yl]phenyl]piperidine-4-carbaldehyde (80 mg, 187.12 μmol, 92.3% yield, N/A purity) as a brown solid which was used in the next step without further purification. LC-MS (ESI⁺) m/z 428.3 [M+H]⁺.

Step 4: To a solution of 1-[4-[(1S,2R)-6-hydroxy-2-(4-hydroxyphenyl)tetralin-1-yl]phenyl]piperidine-4-carbaldehyde (80 mg, 187.12 μmol, N/A purity, 1 eq) and (38)-3-(1-oxo-5-piperazin-1-yl-isoindolin-2-yl)piperidine-2,6-dione (103.03 mg, 205.83 μmol, 1.1 eq, TsOH) in DCM (2.5 mL) and MeOH (2.5 mL), after stirred 30 min was added NaBH(OAc) 3 (118.98 mg, 561.36 μmol, 3 eq). The mixture was stirred at 25° C. for 12 h. The reaction mixture was concentrated to yield a residue. The residue was purified by preparative HPLC (column: Welch Xtimate C18 150*25 mm*5 um; mobile phase: [water (NH₄HCO₃)-ACN]; B %: 43%-63%, 11 min), followed by lyophilization to yield (3S)-3-[5-[4-[[1-[4-[(1S,2R)-6-hydroxy-2-(4-hydroxyphenyl)tetralin-1-yl]phenyl]-4-piperidyl]methyl]piperazin-1-yl]-1-oxo-isoindolin-2-yl]piperidine-2,6-dione (65.4 mg, 86.34 μmol, 46.14% yield, 97.684% purity) as a white solid. LC-MS (ESI⁺) m/z 725.5 [M+H]⁺.

LCMS: calc. for C₄₅H₄₉N₅O₅:739.37, found: [M+H]⁺ 740.5.

HPLC: 97.684% purity at 220 nm.

¹H NMR (400 MHZ, CD₃OD) δ=7.63 (d, J=8.5 Hz, 1H), 7.12-7.04 (m, 2H), 6.71-6.58 (m, 6H), 6.57-6.48 (m, 3H), 6.33 (d, J=8.6 Hz, 2H), 5.09 (dd, J=5.1, 13.4 Hz, 1H), 4.47-4.33 (m, 2H), 4.12 (d, J=5.1 Hz, 1H), 3.57-3.48 (m, 2H), 3.40-3.34 (m, 4H), 3.25-3.19 (m, 1H), 3.01-2.84 (m, 3H), 2.81-2.73 (m, 1H), 2.67-2.54 (m, 6H), 2.46 (dq, J=4.5, 13.2 Hz, 1H), 2.31 (d, J=7.0 Hz, 2H), 2.14 (ddd, J=2.7, 5.2, 7.6 Hz, 2H), 1.87 (br d, J=12.8 Hz, 2H), 1.70 (br d, J=6.9 Hz, 2H), 1.38-1.28 (m, 2H)

Step 5: To a solution of (1R,2S)-1-[4-[4-(dimethoxymethyl)-1-piperidyl]phenyl]-2-(4-hydroxyphenyl)tetralin-6-ol (100 mg, 209.04 μmol, 99% purity, 1 eq) in DCM (2.5 mL) was added TFA (770.00 mg, 6.75 mmol, 0.5 mL, 32.31 eq). The mixture was stirred at 25° C. for 1 h.

The reaction mixture was quenched by addition of water (30 mL), extracted with EtOAc (20 mL×3). The combined organic layers were washed with brine (10 mL), dried over Na₂SO₄, filtered and concentrated under reduced pressure to yield 1-[4-[(1R,2S)-6-hydroxy-2-(4-hydroxyphenyl)tetralin-1-yl]phenyl]piperidine-4-carbaldehyde (80 mg, 187.12 μmol, 89.5% yield, N/A purity) as a brown solid which was used in the next step without further purification. LC-MS (ESI⁺) m/z 428.3 [M+H]⁺.

Step 6: To a solution of 1-[4-[(1R,2S)-6-hydroxy-2-(4-hydroxyphenyl)tetralin-1-yl]phenyl]piperidine-4-carbaldehyde (80.00 mg, 187.12 μmol, N/A purity, 1 eq) and (35)-3-(1-oxo-5-piperazin-1-yl-isoindolin-2-yl)piperidine-2,6-dione (103.03 mg, 205.83 μmol, 1.1 eq, TsOH) in DCM (2.5 mL) and MeOH (2.5 mL), after stirred 30 min was added NaBH(OAc) 3 (118.98 mg, 561.36 μmol, 3 eq). The mixture was stirred at 25° C. for 12 h. The residue was filtered and purified by preparative HPLC (column: Welch Xtimate C18 150*25 mm*5 um; mobile phase: [water (NH₄HCO₃)-ACN]; B %: 43%-63%, 11 min), followed by lyophilization to yield (3S)-3-[5-[4-[[1-[4-[(1R,2S)-6-hydroxy-2-(4-hydroxyphenyl)tetralin-1-yl]phenyl]-4-piperidyl]methyl]piperazin-1-yl]-1-oxo-isoindolin-2-yl]piperidine-2,6-dione (61.6 mg, 83.07 μmol, 44.39% yield, 99.779% purity) as a white solid. LC-MS (ESI⁺) m/z 740.5 [M+H]⁺.

LCMS: calc. for C₄₅H₄₉N₅O₅:739.37, found: [M+H]⁺ 740.5.

HPLC: 99.779% purity at 220 nm.

¹H NMR (400 MHz, CD₃OD) δ=7.63 (d, J=8.3 Hz, 1H), 7.11-7.05 (m, 2H), 6.70-6.59 (m, 6H), 6.57-6.49 (m, 3H), 6.34 (d, J=8.6 Hz, 2H), 5.09 (dd, J=5.0, 13.4 Hz, 1H), 4.46-4.33 (m, 2H), 4.12 (d, J=5.2 Hz, 1H), 3.53 (br d, J=10.4 Hz, 2H), 3.39-3.35 (m, 4H), 3.23 (br d, J=11.1 Hz, 1H), 3.01-2.85 (m, 3H), 2.82-2.73 (m, 1H), 2.64-2.54 (m, 6H), 2.46 (dq, J=4.5, 13.2 Hz, 1H), 2.31 (d, J=6.9 Hz, 2H), 2.20-2.09 (m, 2H), 1.87 (br d, J=13.7 Hz, 2H), 1.70 (br d, J=7.2 Hz, 2H), 1.38-1.29 (m, 2H), 1.40-1.27 (m, 1H).

### Example 209. Preparation of (I-127) (3S)-3-[5-[4-[[1-[4-[(1S,2R)-6-hydroxy-2-(3-hydroxyphenyl)tetralin-1-yl]phenyl]-4-piperidyl]methyl]piperazin-1-yl]-1-oxo-isoindolin-2-yl]piperidine-2,6-dione

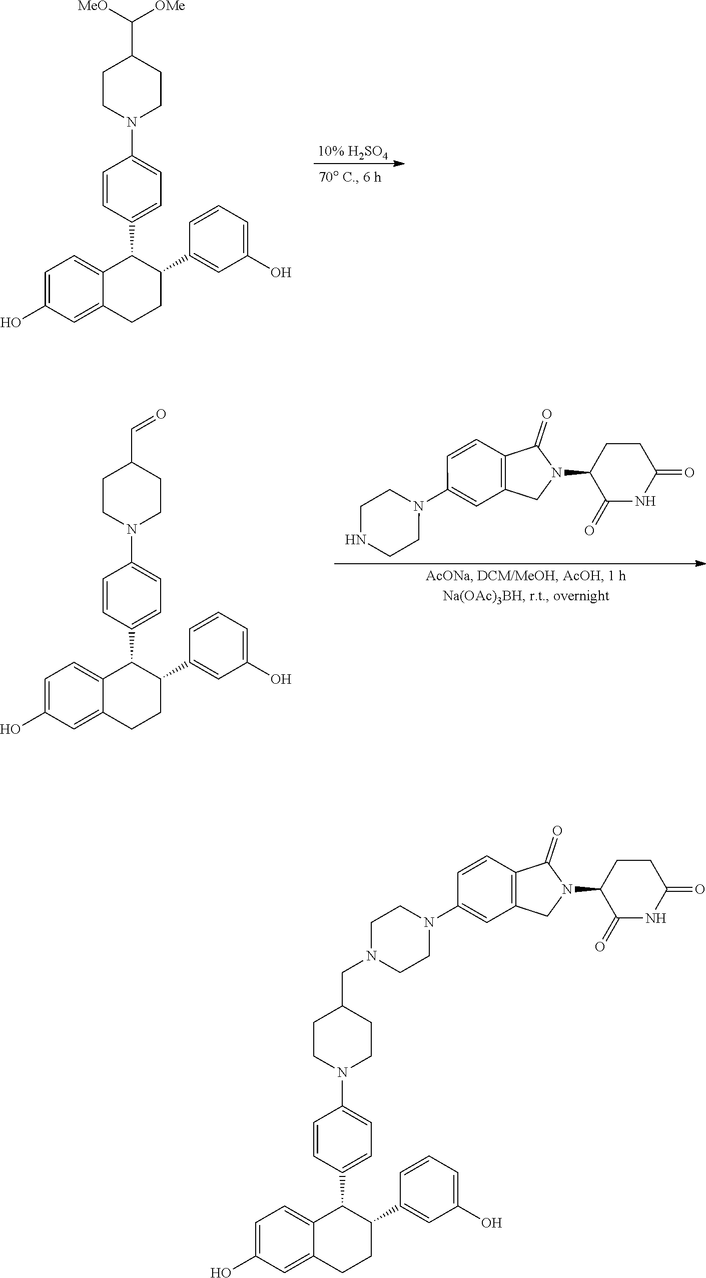 [SMILES: COC(OC)C1CCN(c2ccc([C@H]3c4ccc(O)cc4CC[C@H]3c3cccc(O)c3)cc2)CC1 ; "10% H2SO4" ; O=CC1CCN(c2ccc([C@H]3c4ccc(O)cc4CC[C@H]3c3cccc(O)c3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7c7cccc(O)c7)cc6)CC5)CC4)ccc3C2=O)C(=O)N1]

Step 1: To a solution of ((5S,6R)-5-(4-(4-(dimethoxymethyl)piperidin-1-yl)phenyl)-6-(3-hydroxyphenyl)-5,6,7,8-tetrahydronaphthalen-2-ol (90 mg, 1 Eq, 0.19 mmol) in THF (3 mL) was added sulfuric acid (0.19 g, 3 mL, 10% Wt, 1 Eq, 0.19 mmol). The mixture was stirred at 70° C. for 6 hr. LCMS showed 77.035% desired MS. The reaction was adjust to pH=7 with NaHCO₃, then 50 mL water was added, and the mixture was extracted with dichloromethane (100 mL×2). The combined organic layers were washed with brine (50 mL), dried over anhydrous Na₂SO₄, filtered and concentrated to dryness in vacuo to give a yellow oil. The yellow oil was concentrated in vacuo to give 1-(4-((1S,2R)-6-hydroxy-2-(3-hydroxyphenyl)-1,2,3,4-tetrahydronaphthalen-1-yl)phenyl)piperidine-4-carbaldehyde (80 mg, 0.17 mmol) as a yellow solid. LC-MS (ESI⁺) m/z: 446.3 (M+H₂O)⁺

Step 2: To a solution of 1-[4-[(1S,2R)-6-hydroxy-2-(3-hydroxyphenyl)tetralin-1-yl]phenyl]piperidine-4-carbaldehyde (80 mg, 187.12 μmol, 1 eq) and (3S)-3-(1-oxo-5-piperazin-1-yl-isoindolin-2-yl)piperidine-2,6-dione (93.67 mg, 187.12 μmol, 1 eq, p-TSA) in DCM (4 mL) and MeOH (4 mL) was added sodium acetate (46.05 mg, 561.36 μmol, 3 eq). The mixture was stirred at 25° C. for 1 b. Then the mixture was added sodium triacetoxyboranuide (79.32 mg, 374.24 μmol, 2 eq) and acetic acid (33.71 mg, 561.36 μmol, 32.1 uL, 3 eq). The mixture was stirred at 25° C. for 16 hr. The reaction mixture was diluted with 15 mL H₂O and extracted with DCM (50 mL*2). The combined organic layers were washed with brine 50 mL (25 mL*2), dried over Na₂SO₄, filtered and concentrated under reduced pressure to give a residue. The residue was purified by prep.HPLC (column: 2_Phenomenex Gemini C18 75*40 mm*3 um; mobile phase: [water(FA)-ACN]; B %: 17%-37%, 7 min). Compound (3S)-3-[5-[4-[[1-[4-[(1S,2R)-6-hydroxy-2-(3-hydroxyphenyl)tetralin-1-yl]phenyl]-4-piperidyl]methyl]piperazin-1-yl]-1-oxo-isoindolin-2-yl]piperidine-2,6-dione (28 mg, 37.84 μmol, 20.22% yield) was obtained as a white solid.

LC-MS (ESI+) m/z: 740.5 (M+H)+

HPLC: 97.915%, purity at 220 nm.

SFC Rt: 2.605 min; Area, 76.253%; method: ID_MeOH_DEA_MeCN_50_1ML_10 MlN_10 CM.

¹H NMR (400 MHZ, DMSO-d₆) δ ppm 10.92-10.96 (m, 1H) 8.92-9.16 (m, 1H) 8.16-8.21 (m, 0.777H) 7.45-7.58 (m, 1H) 7.03-7.12 (m, 2H) 6.88-6.99 (m, 1H) 6.62-6.68 (m, 1H) 6.45-6.60 (m, 5H) 6.30-6.37 (m, 1H) 6.20-6.28 (m, 3H) 5.00-5.09 (m, 1H) 4.30-4.38 (m, 1H) 4.17-4.25 (m, 1H) 4.07-4.14 (m, 1H) 3.49-3.56 (m, 2H) 3.29 (br d, J=3.93 Hz, 6H) 3.14-3.23 (m, 2H) 2.84-2.96 (m, 3H) 2.66-2.71 (m, 1H) 2.55-2.64 (m, 2H) 2.31-2.40 (m, 2H) 2.17-2.24 (m, 2H) 1.92-2.09 (m, 2H) 1.58-1.81 (m, 4H) 1.09-1.24 (m, 2H)

### Example 210. Preparation of (I-126) (3S)-3-[5-[4-[[1-[4-[(1R,2S)-6-hydroxy-2-(3-hydroxyphenyl)tetralin-1-yl]phenyl]-4-piperidyl]methyl]piperazin-1-yl]-1-oxo-isoindolin-2-yl]piperidine-2,6-dione

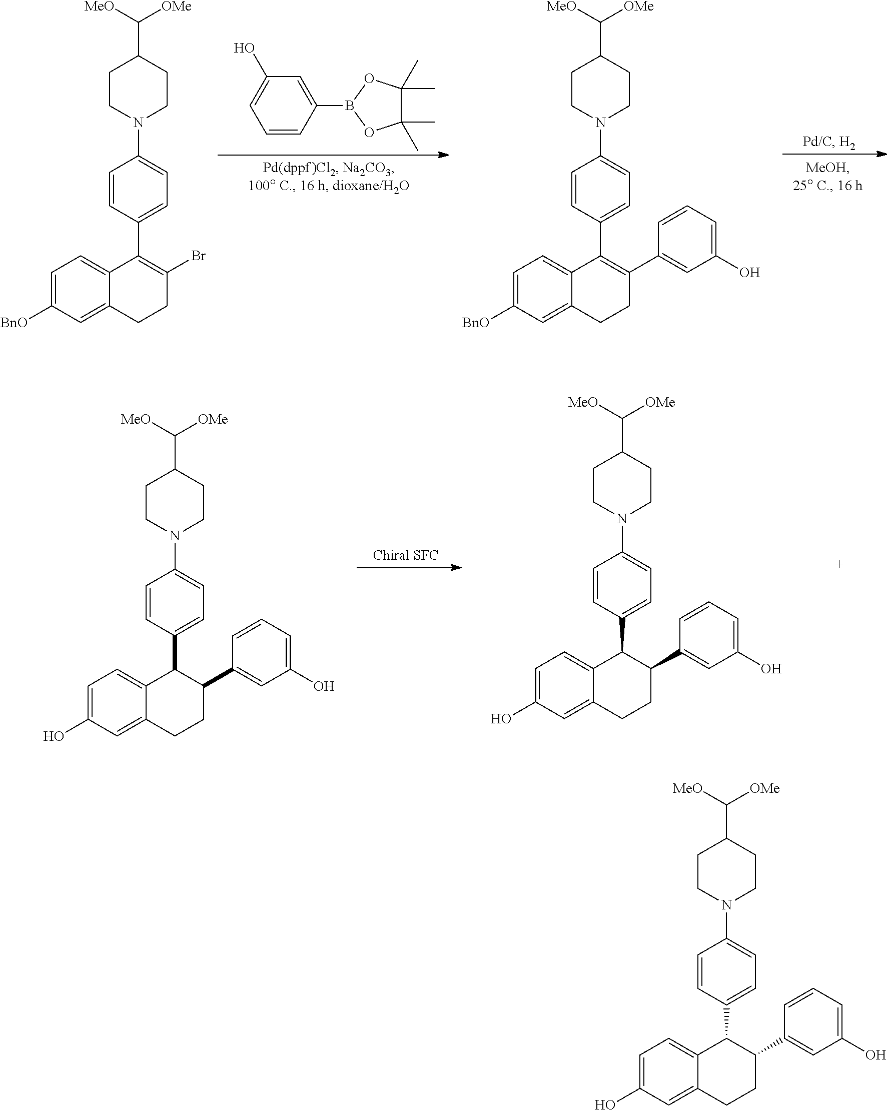 [SMILES: COC(OC)C1CCN(c2ccc(C3=C(Br)CCc4cc(OCc5ccccc5)ccc43)cc2)CC1 ; CC1(C)OB(c2cccc(O)c2)OC1(C)C ; COC(OC)C1CCN(c2ccc(C3=C(c4cccc(O)c4)CCc4cc(OCc5ccccc5)ccc43)cc2)CC1 ; COC(OC)C1CCN(c2ccc([C@@H]3c4ccc(O)cc4CC[C@@H]3c3cccc(O)c3)cc2)CC1 ; COC(OC)C1CCN(c2ccc([C@@H]3c4ccc(O)cc4CC[C@@H]3c3cccc(O)c3)cc2)CC1 ; COC(OC)C1CCN(c2ccc([C@H]3c4ccc(O)cc4CC[C@H]3c3cccc(O)c3)cc2)CC1]

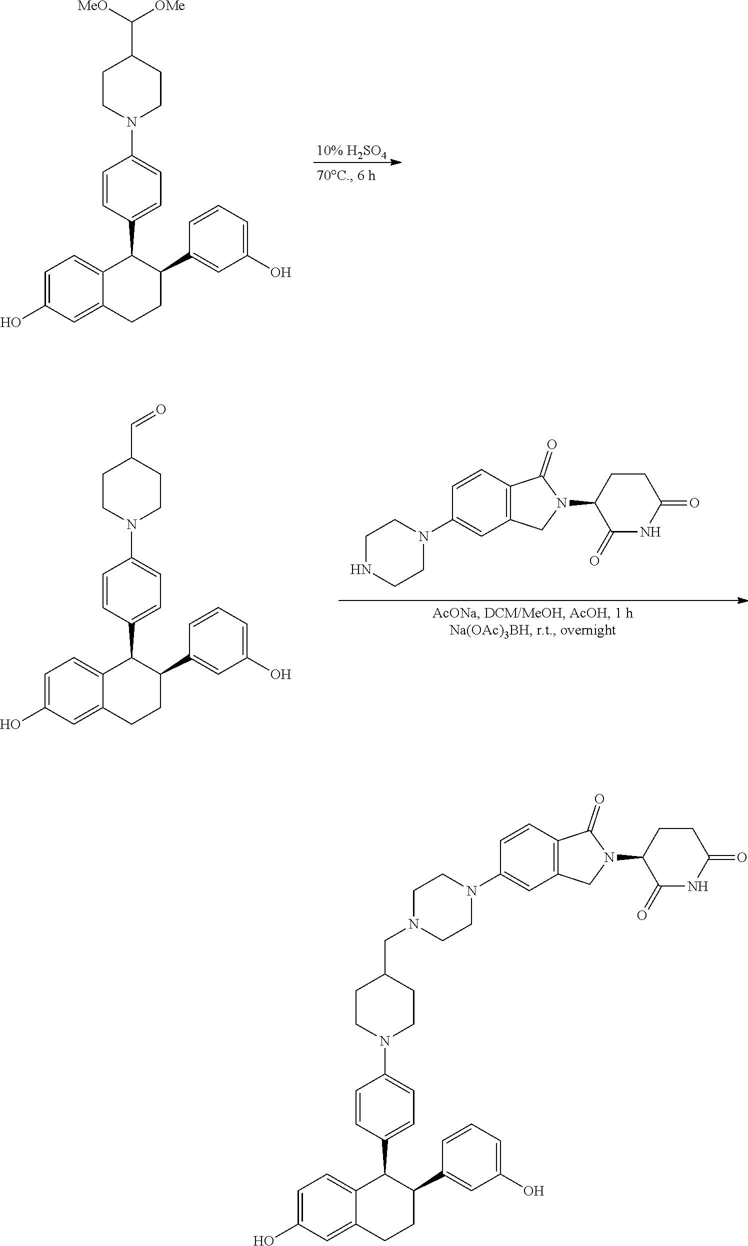 [SMILES: COC(OC)C1CCN(c2ccc([C@@H]3c4ccc(O)cc4CC[C@@H]3c3cccc(O)c3)cc2)CC1 ; O=CC1CCN(c2ccc([C@@H]3c4ccc(O)cc4CC[C@@H]3c3cccc(O)c3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc(O)c7)cc6)CC5)CC4)ccc3C2=O)C(=O)N1]

Step 1: A mixture of sodium carbonate (232 mg, 3 Eq, 2.19 mmol), Pd(dppf)Cl₂ (59.6 mg, 0.1 Eq, 72.9 μmol), 3-(4,4,5,5-tetramethyl-1,3,2-dioxaborolan-2-yl)phenol (193 mg, 1.2 Eq, 875 μmol), 1-(4-(6-(benzyloxy)-2-bromo-3,4-dihydronaphthalen-1-yl)phenyl)-4-(dimethoxymethyl)piperidine (400 mg, 1 Eq, 729 μmol) in 1,4-Dioxane (8 mL) and H₂O (2 mL) was degassed and purged with N₂ for 3 times, and then the mixture was stirred at 100° C. for 16 hr under N₂ atmosphere. LCMS showed 38.6% desired MS. TLC (petroleum ether:ethyl acetate=5:1, R_{f}=0.5 UV) showed one main new spot was observed. The reaction was quenched with water (50 mL) and extracted with ethyl acetate (150 mL*2). The organic layer was washed with brine (20 mL) and dried over Na₂SO₄ and concentrated in vacuo to give a black solid. The black solid was subjected to column chromatography over silica gel (gradient elution: 0-100% EtOAc). The desired fractions were collected, and concentrated to dryness in vacuo to give 3-(6-(benzyloxy)-1-(4-(4-(dimethoxymethyl)piperidin-1-yl)phenyl)-3,4-dihydronaphthalen-2-yl)phenol (300 mg, 509 μmol, 69.8%, 95.323% Purity) as a yellow solid. LC-MS (ESI⁺) m/z: 562.4 (M+H)⁺

Step 2: To a solution of 3-(6-(benzyloxy)-1-(4-(4-(dimethoxymethyl)piperidin-1-yl)phenyl)-3,4-dihydronaphthalen-2-yl)phenol (300 mg, 1 Eq, 534 μmol) in MeOH (6 mL) was added Pd/C (300 mg, 10% Wt, 0.528 Eq, 282 μmol) under N₂ atmosphere. The suspension was degassed and purged with dihydrogen (1.08 mg, 1 Eq, 534 μmol) for 10 mins. The mixture was stirred under 15 Psi dihydrogen (1.08 mg, 1 Eq, 534 μmol) at 50° C. for 16 hr. LCMS showed 90% desired MS. The reaction was filtrate and concentrated. The residue was further separated by HPLC (condition: column: DAICEL CHIRALPAK IG (250 mm*30 mm, 10 um); mobile phase: 0.1% NH₃HO₂ ETOH; B %: 40%-40%, min) to give desired 5-(4-(4-(dimethoxymethyl)piperidin-1-yl)phenyl)-6-(3-hydroxyphenyl)-5,6,7,8-tetrahydronaphthalen-2-ol (220 mg, 465 μmol, 87.0%) as a yellow solid. LC-MS (ESI⁺) m/z: 474.3 (M+H)⁺.

Step 3: 220 mg of 5-(4-(4-(dimethoxymethyl)piperidin-1-yl)phenyl)-6-(3-hydroxyphenyl)-5,6,7,8-tetrahydronaphthalen-2-ol was further separated by SFC (condition: column: DAICEL CHIRALPAK IG (250 mm*30 mm, 10 um); mobile phase: 0.1% NH₃·H₂O ETOH; B %: 40%-40%, 80 ml/min) to give desired (5R,6S)-5-(4-(4-(dimethoxymethyl)piperidin-1-yl)phenyl)-6-(3-hydroxyphenyl)-5,6,7,8-tetrahydronaphthalen-2-ol (70 mg, 0.15 mmol) as a yellow solid. LC-MS (ESI⁺) m/z: 474.3 (M+H)⁺ and (5S,6R)-5-(4-(4-(dimethoxymethyl)piperidin-1-yl)phenyl)-6-(3-hydroxyphenyl)-5,6,7,8-tetrahydronaphthalen-2-ol (90 mg, 0.19 mmol) as a yellow solid. LC-MS (ESI⁺) m/z: 474.3 (M+H)⁺.

Step 4: To a solution of ((5R,6S)-5-(4-(4-(dimethoxymethyl)piperidin-1-yl)phenyl)-6-(3-hydroxyphenyl)-5,6,7,8-tetrahydronaphthalen-2-ol (70 mg, 1 Eq, 0.15 mmol) in THF (3 mL) was added sulfuric acid (0.14 g, 3 mL, 10% Wt, 1 Eq, 0.15 mmol). The mixture was stirred at 70° C. for 6 hr. LCMS showed 91.9% desired MS. The reaction was adjust to pH˜=7 with NaHCO₃, then 50 mL water was added, and the mixture was extracted with DCM (100 mL×2). The combined organic layers were washed with brine (10 mL), dried over anhydrous Na₂SO₄, filtered and concentrated to dryness in vacuo to give a yellow oil. The yellow oil was concentrated in vacuo to give 1-(4-((1R, 2S)-6-hydroxy-2-(3-hydroxyphenyl)-1,2,3,4-tetrahydronaphthalen-1-yl)phenyl)piperidine-4-carbaldehyde (65 mg, 0.15 mmol, 100%) as a yellow solid. LC-MS (ESI⁺) m/z: 405.2 (M+H)⁺

Step 5: To a solution of 1-[4-[(1R,2S)-6-hydroxy-2-(3-hydroxyphenyl)tetralin-1-yl]phenyl]piperidine-4-carbaldehyde (70 mg, 163.73 μmol, 1 eq) and (3S)-3-(1-oxo-5-piperazin-1-yl-isoindolin-2-yl)piperidine-2,6-dione (81.96 mg, 163.73 μmol, 1 eq, p-TSA) in DCM (4 mL) and MeOH (4 mL) was added sodium; acetate (40.29 mg, 491.19 μmol, 3 eq). The mixture was stirred at 25° C. for 1 hr. Then the mixture was added sodium triacetoxyboranuide (69.40 mg, 327.46 μmol, 2 eq) and acetic acid (29.50 mg, 491.19 μmol, 28.09 uL, 3 eq). The mixture was stirred at 25° C. overnight. LCMS showed desired MS was detected. The reaction mixture was diluted with 20 mL H₂O and extracted with DCM (50 mL*2). The combined organic layers were washed with brine 40 mL (20 mL*2), dried over Na₂SO₄, filtered and concentrated under reduced pressure to give a residue. The residue was purified by prep.HPLC (column: 2 Phenomenex Gemini C18 75*40 mm*3 um; mobile phase: [water(FA)-ACN]; B %: 15%-45%, 7 min). Compound (3S)-3-[5-[4-[[1-[4-[(1R,2S)-6-hydroxy-2-(3-hydroxyphenyl)tetralin-1-yl]phenyl]-4-piperidyl]methyl]piperazin-1-yl]-1-oxo-isoindolin-2-yl]piperidine-2,6-dione (70 mg, 94.61 μmol, 57.78% yield) was obtained as a white solid. LC-MS (ESI+) m/z: 740.4 (M+H)⁺

HPLC: 98.082%, purity at 220 nm.

SFC: Rt: 2.639 min; Area, 71.130%; method:

ID_MeOH_DEA_MeCN_50_1ML_10 MlN_10 CM.

¹H NMR (400 MHZ, DMSO-d₆) δ ppm 10.92-10.97 (m, 1H) 8.99-9.15 (m, 1H) 8.19-8.25 (m, 0.366H) 7.45-7.56 (m, 1H) 7.02-7.10 (m, 2H) 6.91 (s, 1H) 6.62-6.68 (m, 1H) 6.45-6.60 (m, 5H) 6.31-6.36 (m, 1H) 6.20-6.28 (m, 3H) 5.01-5.09 (m, 1H) 4.30-4.38 (m, 1H) 4.16-4.26 (m, 1H) 4.07-4.14 (m, 1H) 3.49-3.58 (m, 2H) 3.29 (br d, J=3.46 Hz, 6H) 3.21 (br s, 2H) 2.83-3.02 (m, 3H) 2.65-2.71 (m, 1H) 2.55-2.63 (m, 2H) 2.30-2.39 (m, 2H) 2.15-2.23 (m, 2H) 1.91-2.10 (m, 2H) 1.59-1.83 (m, 4H) 1.10-1.27 (m, 2H).

### Example 211. Preparation of (I-121) (S)-3-(5-(4-((1-(4-((1R,2R)-2-(cyclopentylmethyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

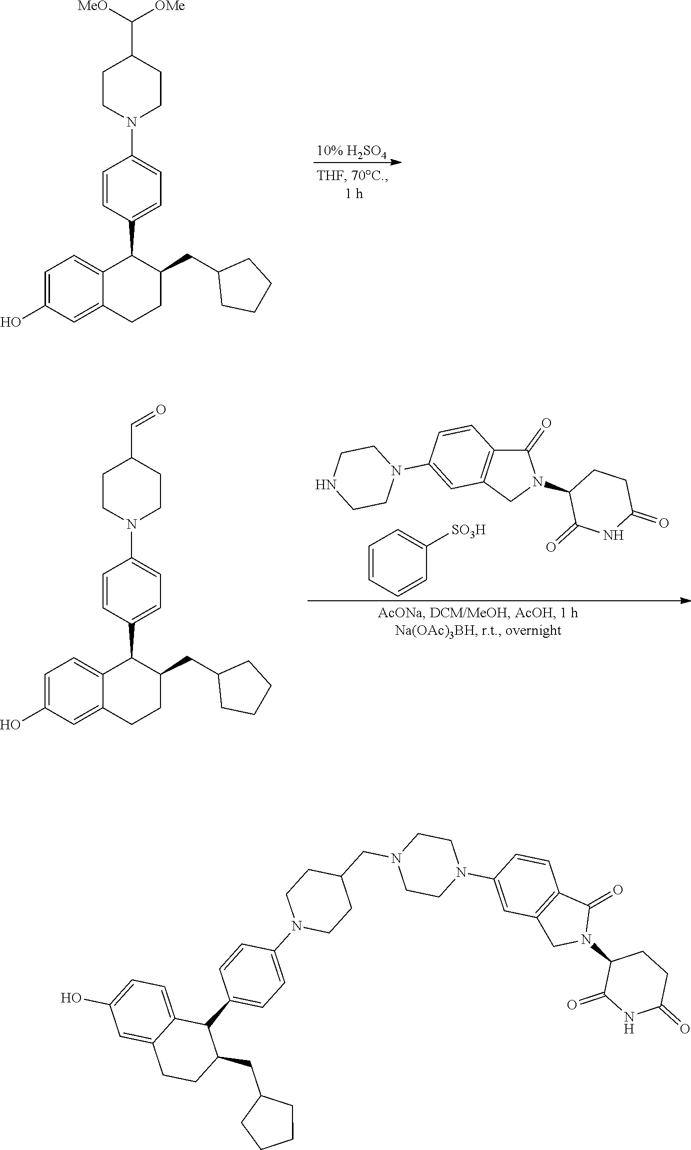

Step 1: To a solution of (5R,6R)-6-(cyclopentylmethyl)-5-(4-(4-(dimethoxymethyl)piperidin-1-yl)phenyl)-5,6,7,8-tetrahydronaphthalen-2-ol (65 mg, 0.14 mmol) in THF (1 mL) and 10% H₂SO₄ (1 mL) was stirred at 70° C. for 1 h. The reaction mixture was quenched by addition 10 mL NaHCO₃ at 0° C., then extracted with ethyl acetate (20 mL×2). The combined organic layers were washed with 20 mL brine dried over Na₂SO₄, filtered and concentrated under reduced pressure to give 1-(4-((1R,2R)-2-(cyclopentylmethyl)-6-hydroxy-1,2,3,4-tetrahydronaphthalen-1-yl)phenyl)piperidine-4-carbaldehyde (60 mg, 100% Yield) as a yellow solid. LC-MS (ESI+) m/z: 436.2 (M+18)⁺.

Step 2: A mixture of 1-(4-((1R,2R)-2-(cyclopentylmethyl)-6-hydroxy-1,2,3,4-tetrahydronaphthalen-1-yl)phenyl)piperidine-4-carbaldehyde (60 mg, 0.14 mmol), (S)-3-(1-oxo-5-(piperazin-1-yl)isoindolin-2-yl)piperidine-2,6-dione benzenesulfonate (0.10 g, 0.22 mmol) and Sodium acetate (59 mg, 0.72 mmol) and Acetic acid (17 mg, 0.29 mmol) in THF (2 mL) and MeOH (2 mL) at 25° C. for 1 h, then Sodium triacetoxyborohydride (61 mg, 0.29 mmol) was added to the mixture and was stirred at 25° C. for 16 h. The mixture was added H₂O (20 mL), extracted with ethyl acetate (20 mL×2). The combined extracts was dried over anhydrous Na₂SO₄, filtered and concentrated to dryness in vacuo to give a residue. The residue was purified by prep-HPLC (column: O-Welch C18 150*30 mm*5 um; mobile phase: [water(FA)-ACN]; B % 20%-60%, 10 min) to give (S)-3-(5-(4-((1-(4-((1R,2R)-2-(cyclopentylmethyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (46.2 mg, 44% Yield) as a white solid. LC-MS (ESI+) m/z: 730.3 (M+H)+.

LCMS: calc. for C₄₅H₅₅N₅O₄: 729.97, found: [M+H]⁺ 730.3.

HPLC: 100% purity at 220 nm.

¹HNMR (400 MHz, DMSO-d₆) δ=10.95 (s, 1H), 9.02 (br s, 1H), 8.14 (s, 1H), 7.53 (d, J=8.6 Hz, 1H), 7.10-7.03 (m, 2H), 6.77 (s, 4H), 6.61 (d, J=8.3 Hz, 1H), 6.52 (d, J=2.3 Hz, 1H), 6.43 (dd, J=2.4, 8.3 Hz, 1H), 5.05 (dd, J=5.0, 13.4 Hz, 1H), 4.33 (d, J=17.2 Hz, 1H), 4.26-4.17 (m, 1H), 3.89 (d, J=4.8 Hz, 1H), 3.60 (br s, 2H), 3.30 (br d, J=3.7 Hz, 6H), 2.96-2.71 (m, 4H), 2.60 (br d, J=11.1 Hz, 5H), 2.44-2.35 (m, 1H), 2.24 (br d, J=6.6 Hz, 2H), 2.00-1.91 (m, 2H), 1.80 (br d, J=11.8 Hz, 3H), 1.70 (br d, J=5.6 Hz, 3H), 1.61-1.44 (m, 5H), 1.27-1.14 (m, 3H), 0.99 (br s, 2H), 0.86-0.75 (m, 1H)

SFC: retention time, 2.873 min; Arca, 77.429%;

### Example 212. Preparation of (I-120) (S)-3-(5-(4-((1-(4-((1S,2S)-2-(cyclopentylmethyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

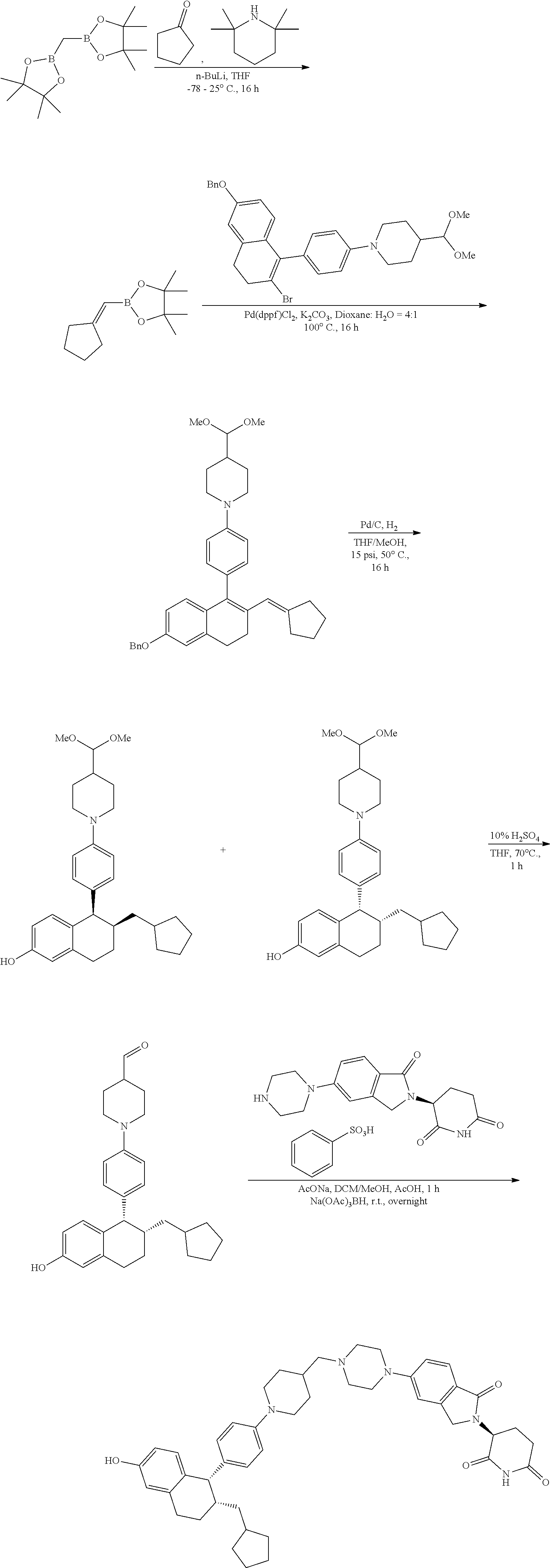

Step 1: A mixture of 2,2,6,6 TetraMethyl Piperidine (1.27 g, 1.52 mL, 8.96 mmol) was dissolved in THF (20 mL) and cooled to −30° C. under N₂ atmosphere. n-butyllithium (574 mg, 3.58 mL, 8.96 mmol) was added dropwise, and the reaction mixture was stirred at the same temperature for 30 min. Next, the reaction was cooled to −78° C., and a solution of bis(4,4,5,5-tetramethyl-1,3,2-dioxaborolan-2-yl)methane (2 g, 7.46 mmol) in THF (20 mL) was added dropwise. After stirring for 30 min, a solution of cyclopentanone (753 mg, 8.96 mmol) in THF (13 mL) was added dropwise at −78° C. The reaction mixture was allowed to slowly warm up to 25° C., and stirred 12 h. The reaction was quenched by Saturate the ammonium chloride solution (30 mL), then stirred for 0.5 h. water (100 mL) was added before the mixture extracted with ethyl acetate (100 mL×3). The organic layers was dried over anhydrous sodium sulfate, filtered and concentrated under reduced pressure to get a yellow oil for next step without purification.

Step 2: [1, l′-Bis(diphenylphosphino)ferrocene]dichloropalladium(II) Complex (52.1 mg, 63.8 μmol) was added to the mixture of 1-(4-(6-(benzyloxy)-2-bromo-3,4-dihydronaphthalen-1-yl)phenyl)-4-(dimethoxymethyl)piperidine (350 mg, 638 μmol), 2-(cyclopentylidenemethyl)-4,4,5,5-tetramethyl-1,3,2-dioxaborolane (700 mg, 3.36 mmol) and Potassium carbonate (265 mg, 112 μL, 1.91 mmol) in Dioxane (10 mL) and H₂O (2 mL) at r.t. N₂ was bubbled into the mixture for 5 min. The reaction mixture was heated at 100° C. for 16 h. The mixture was added H₂O (100 mL), extracted with ethyl acetate (100 mL×2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 80/20). The pure fractions were collected and concentrated to dryness in vacuo to give 1-(4-(6-(benzyloxy)-2-(cyclopentylidenemethyl)-3,4-dihydronaphthalen-1-yl)phenyl)-4-(dimethoxymethyl)piperidine (350 mg, 94.4% Yield) as a yellow solid. LC-MS (ESI⁺) m/z: 550.3 (M+H)⁺.

Step 3: A mixture of 1-(4-(6-(benzyloxy)-2-(cyclopentylidenemethyl)-3,4-dihydronaphthalen-1-yl)phenyl)-4-(dimethoxymethyl)piperidine (330 mg, 568 μmol), dihydrogen (1.15 mg, 568 μmol), Pd/C (330 mg, 10% Wt, 310 μmol) in MeOH (S mL) was degassed and purged with H₂ for 3 times, and then the mixture was stirred at 50° C. for 16 h under H₂ atmosphere (15 psi). The mixture was concentrated to dryness in vacuo to give a residue. The residue was subjected to column chromatography over silica gel (petroleum ether/ethyl acetate from 100/0 to 80/20). The pure fractions were collected and concentrated to dryness in vacuo to give a white solid. The white solid was purified by SFC (column: DAICEL CHIRALCEL OJ (250 mm*30 mm, 10 um); mobile phase: [0.1% NH3H2O (ETOH)]; B % 30%-30%) to give The aqueous phase was lyophilized to dryness to give 1-(4-((1R,2R)-6-(benzyloxy)-2-(cyclopentylmethyl)-1,2,3,4-tetrahydronaphthalen-1-yl)phenyl)-4-(dimethoxymethyl)piperidine (65 mg, 21% Yield) and 1-(4-((1S,2S)-6-(benzyloxy)-2-(cyclopentylmethyl)-1,2,3,4-tetrahydronaphthalen-1-yl)phenyl)-4-(dimethoxymethyl)piperidine (65 mg, 21% Yield) as white solids. LC-MS (ESI⁺) m/z: 464.2 (M+H)⁺.

Step 4: To a solution of (5S,6S)-6-(cyclopentylmethyl)-5-(4-(4-(dimethoxymethyl)piperidin-1-yl)phenyl)-5,6,7,8-tetrahydronaphthalen-2-ol (65 mg, 0.14 mmol) in THF (1 mL) and 10% H₂SO₄ (1 mL) was stirred at 70° C. for 1 h. The reaction was adjust to pH˜=8 with aq.NaHCO₃ (5 mL). Then the mixture was added water (15 mL) and extracted with ethyl acetate (20×2 mL). The combined organic layers were washed with brine (20 mL), dried over anhydrous Na₂SO₄, filtered and concentrated to dryness in vacuo to give 1-(4-((1S,2S)-2-(cyclopentylmethyl)-6-hydroxy-1,2,3,4-tetrahydronaphthalen-1-yl)phenyl)piperidine-4-carbaldehyde (60 mg, 100% Yield) as a yellow solid. LC-MS (ESI⁺) m/z: 418.1 (M+H)⁺.

Step 5: A mixture of 1-(4-((1S,2S)-2-(cyclopentylmethyl)-6-hydroxy-1,2,3,4-tetrahydronaphthalen-1-yl)phenyl)piperidine-4-carbaldehyde (60 mg, 0.14 mmol), (S)-3-(1-oxo-5-(piperazin-1-yl)isoindolin-2-yl)piperidine-2,6-dione benzenesulfonate (0.10 g, 0.21 mmol) and Sodium acetate (57 mg, 0.70 mmol) and Acetic acid (17 mg, 0.28 mmol) in THF (2 mL) and MeOH (2 mL) at 25° C. for 1 h, then Sodium triacetoxyborohydride (59 mg, 0.28 mmol) was added to the mixture and was stirred at 25° C. for 16 h. The mixture was added H₂O (10 mL), extracted with ethyl acetate (10 mL×2). The combined extracts was dried over anhydrous Na₂SO₄, filtered and concentrated to dryness in vacuo to give a residue. The residue was purified by prep-HPLC (column: O-Welch C18 150*30 mm*5 um; mobile phase: [water(FA)-ACN]; B % 20%-60%, 8 min) to give (S)-3-(5-(4-((1-(4-((1S,2S)-2-(cyclopentylmethyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (48.4 mg, 47% Yield) as a white solid. LC-MS (ESI+) m/z: 730.2 (M+H)⁺.

LCMS: calc. for C₄₅H₅₅N₅O₄:729.97, found: [M+H]⁺ 730.2.

HPLC: 100% purity at 220 nm.

¹HNMR (400 MHz, DMSO-d₆) δ=10.95 (s, 1H), 9.02 (s, 1H), 8.14 (s, 1H), 7.53 (d, J=8.6 Hz, 1H), 7.10-7.04 (m, 2H), 6.77 (s, 4H), 6.61 (d, J=8.3 Hz, 1H), 6.52 (d, J=2.0 Hz, 1H), 6.43 (dd, J=2.4, 8.2 Hz, 1H), 5.05 (dd, J=4.9, 13.3 Hz, 1H), 4.34 (d, J=17.0 Hz, 1H), 4.25-4.17 (m, 1H), 3.89 (d, J=4.8 Hz, 1H), 3.61 (br d, J=5.1 Hz, 2H), 3.32-3.27 (m, 6H), 2.98-2.69 (m, 3H), 2.63-2.54 (m, 6H), 2.43-2.35 (m, 1H), 2.25 (br s, 2H), 2.01-1.91 (m, 2H), 1.80 (br d, J=12.5 Hz, 3H), 1.70 (br d, J=5.0 Hz, 3H), 1.56 (br s, 3H), 1.48 (br dd, J=4.4, 7.3 Hz, 2H), 1.25-1.15 (m, 3H), 0.99 (br s, 2H), 0.85-0.77 (m, 1H)

SFC: retention time, 2.612 min; Area, 79.202%;

### Example 213. Preparation of (I-129) (R)-3-(5-(4-((1-(4-((1S,2S)-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione

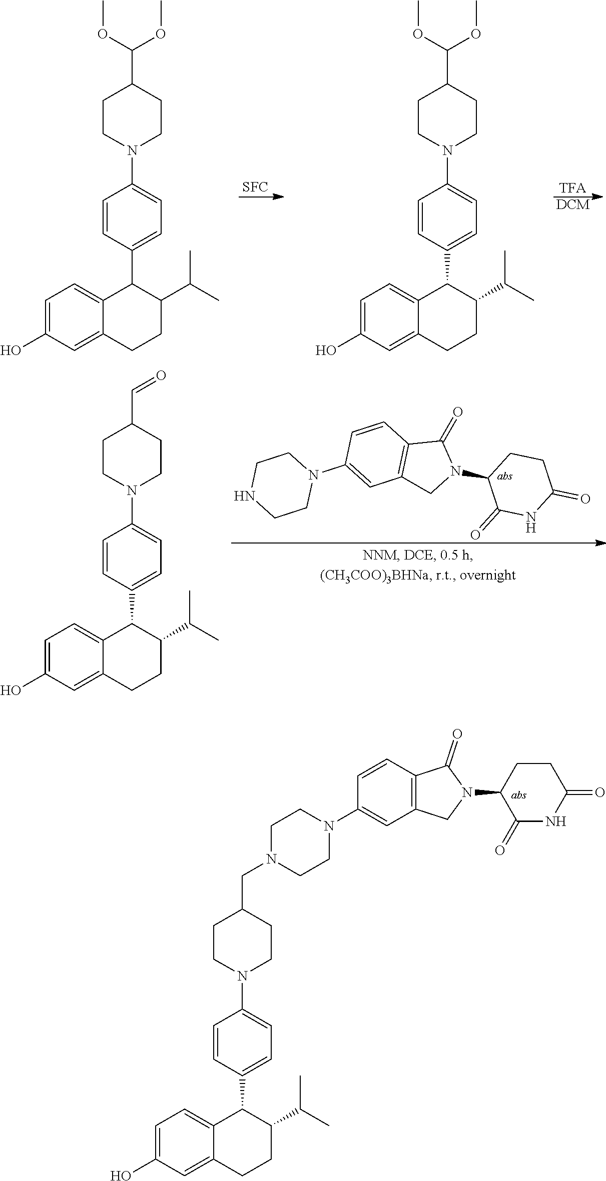

Step 1: The 5-(4-(4-(dimethoxymethyl)piperidin-1-yl)phenyl)-6-isopropyl-5,6,7,8-tetrahydronaphthalen-2-ol (400 mg, 1 eq., 944 μmol) was purified by SFC(Column: DAICEL CHIRALCEL OD-H (250 mm*30 mm, 10 um); Mobile phase: 0.1% NH₃H₂O ETOH; from 35% to 35%; Flow rate: 70 mL/min) to give (5S,6S)-5-(4-(4-(dimethoxymethyl)piperidin-1-yl)phenyl)-6-isopropyl-5,6,7,8-tetrahydronaphthalen-2-ol (130 mg, 307 μmol, 32.5%) as white solid.

LC-MS (ESI⁺) m/z: 424.4 (M+H)⁺.

Step 2: To a solution of (5S,6S)—S-(4-(4-(dimethoxymethyl)piperidin-1-yl)phenyl)-6-isopropyl-5,6,7,8-tetrahydronaphthalen-2-ol (130 mg, 1 eq., 307 μmol) in DCM (4 mL) was added TFA (105 mg, 70.9 μL, 3 eq., 921 μmol). The mixture was stirred at 20° C. for 4 hour. The mixture was concentrated in vacuo to give crude product. No further purification as it is used for the next step directly. LC-MS (ESI+) m/z: 378.2 (M+H)+

Step 3: To a solution of 1-(4-((1S,2S)-6-hydroxy-2-isopropyl-1,2,3,4-tetrahydronaphthalen-1-yl)phenyl)piperidine-4-carbaldehyde (100 mg, 1 eq., 265 μmol) and (R)-3-(1-oxo-5-(piperazin-1-yl)isoindolin-2-yl)piperidine-2,6-dione (113 mg, 1.3 eq., 344 μmol) in DCE (10 mL) was added dropwise 4-methylmorpholine (26.8 mg, 1 eq., 265 μmol) to pH=8 at 20° C. After addition, the mixture was stirred at this temperature for 30 min, and then sodium triacetoxyhydroborate (140 mg, 2.5 eq., 662 μmol) was added dropwise at 20° C. The resulting mixture was stirred at 20° C. for 3 hour. The crude was purified by prep. HPLC together (Boston Green ODS 150*30 mm*5 um, water(FA)-ACN as a mobile phase, from 20% to 30%, Gradient Time (min): 12, Flow Rate (ml/min): 25) to give (R)-3-(5-(4-((1-(4-((1S,2S)-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione (9.3 mg, 13 μmol, 5.1%) as a white solid.

LC-MS (ESI+) m/z: 690.3 (M+H)+¹H NMR (500 MHZ, METHANOL-d₄) δ: ppm 8.26-8.41 (m, 1H), 7.65 (d, J=8.54 Hz, 1H), 7.09-7.13 (m, 2H), 6.91-6.94 (m, 2H), 6.85-6.89 (m, 2H), 6.66 (d, J=8.39 Hz, 1H), 6.56 (d, J=2.29 Hz, 1H), 6.45 (dd, J=8.32, 2.67 Hz, 1H), 5.10 (br d, J=8.24 Hz, 1H), 4.32-4.46 (m, 2H), 4.12 (d, J=3.97 Hz, 1H), 3.62 (br d, J=11.90 Hz, 2H), 3.44 (br s, 4H), 2.88-2.96 (m, 2H), 2.83 (br s, 4H), 2.74-2.78 (m, 1H), 2.67 (brt, J=11.98 Hz, 2H), 2.49-2.55 (m, 2H), 2.46 (dd, J=13.28, 4.58 Hz, 1H), 2.12-2.18 (m, 1H), 1.91 (br d, J=12.36 Hz, 2H), 1.79 (br d, J=12.21 Hz, 2H), 1.57-1.65 (m, 2H), 1.32-1.41 (m, 3H), 1.08 (d, J=6.56 Hz, 3H), 0.64 (d, J=6.71 Hz, 3H).

### Example 214. Preparation of (I-128) (R)-3-(5-(4-((1-(4-((1R,2R)-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione

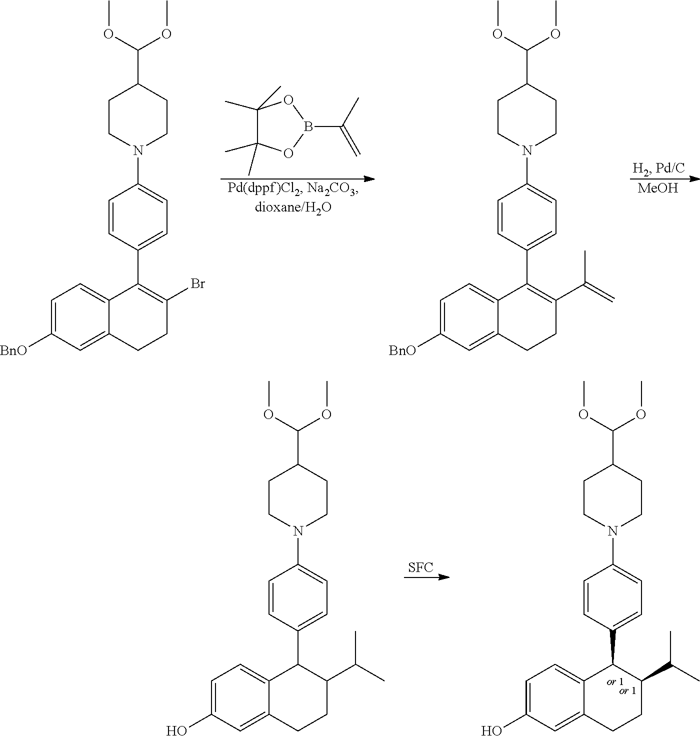

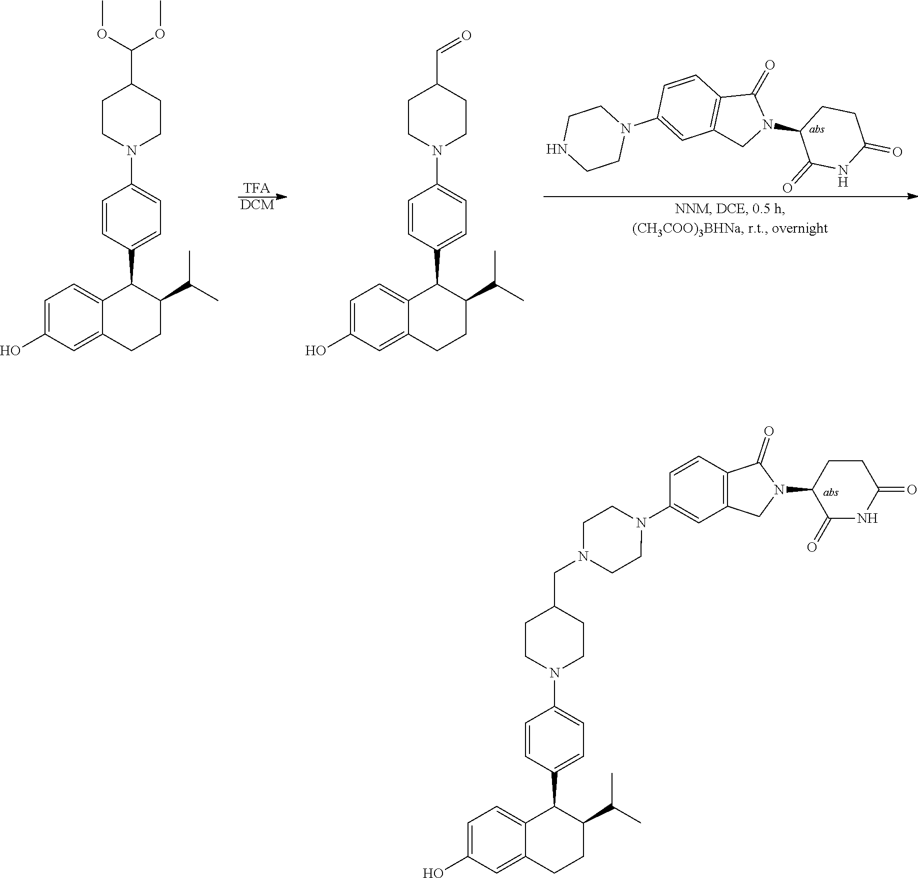

Step 1: A mixture of Isopropenylboronicacidpinacolester (0.4 g, 0.4 mL, 1.3 eq., 2 mmol), 1-(4-(6-(benzyloxy)-2-bromo-3,4-dihydronaphthalen-1-yl)phenyl)-4-(dimethoxymethyl)piperidine (1 g, 1 eq., 2 mmol), Na₂CO₃ (0.6 g, 3 eq., 5 mmol) and dppf (0.1 g, 0.1 eq., 0.2 mmol) in Dioxane (15 mL) and water (3 mL) was degassed and purged with N₂ for 3 times, and then the mixture was stirred at 80° C. for 8 hour under N₂ atmosphere. The reaction mixture was quenched by addition EA 10 mL at 20° C., and then diluted with water 30 mL and extracted with EA 30 mL (10 mL*3). The combined organic layers were washed with EA 30 mL (10 mL*3), dried over Na₂SO₄, filtered and concentrated under reduced pressure to give a residue. The crude material was purified on silica gel column chromatography (from PE/EtOAc=1/0 to 3/2) to give 1-(4-(6-(benzyloxy)-2-(prop-1-en-2-yl)-3,4-dihydronaphthalen-1-yl)phenyl)-4-(dimethoxymethyl)piperidine (0.6 g, 1 mmol, 60%) as yellow oil.

LC-MS (ESI⁺) m/z: 510.3 (M+H)⁺.

¹H NMR (400 MHZ, DMSO-d₆) δ: ppm 7.28-7.47 (m, 5H), 6.84-7.00 (m, 5H), 6.82-6.84 (m, 1H), 6.68 (dd, J=8.52, 2.56 Hz, 1H), 6.39-6.53 (m, 1H), 5.06 (s, 2H), 4.59-4.78 (m, 2H), 4.10 (d, J=6.44 Hz, 1H), 3.71 (br d, J=12.04 Hz, 2H), 3.28 (s, 6H), 2.71-2.82 (m, 2H), 2.60 (br t, J=11.50 Hz, 2H), 2.31-2.48 (m, 2H), 1.70-1.77 (m, 3H), 1.56 (s, 2H), 1.37-1.41 (m, 1H), 1.15-1.23 (m, 2H).

Step 2: To a solution of 1-(4-(6-(benzyloxy)-2-(prop-1-en-2-yl)-3,4-dihydronaphthalen-1-yl)phenyl)-4-(dimethoxymethyl)piperidine (600 mg, 1 eq., 1.18 mmol) in MeOH (10 mL) was added Pd/C (10%, 0.5 g) under N₂ atmosphere. The suspension was degassed and purged with H₂ for 3 times. The mixture was stirred under H₂ (1 atm.) at 20° C. for 12 hour. The mixture was filtered and the filtrate was concentrated. The crude material was purified on silica gel column chromatography (from pure PE to PE/EtOAc=5/1) to give 5-(4-(4-(dimethoxymethyl)piperidin-1-yl)phenyl)-6-isopropyl-5,6,7,8-tetrahydronaphthalen-2-ol (400 mg, 944 μmol, 80.2%) as a yellow solid.

LC-MS (ESI⁺) m/z: 424.3 (M+H)⁺.

Step 3: The 5-(4-(4-(dimethoxymethyl)piperidin-1-yl)phenyl)-6-isopropyl-5,6,7,8-tetrahydronaphthalen-2-ol (400 mg, 1 eq., 944 μmol) was purified by SFC(Column: DAICEL CHIRALCEL OD-H (250 mm*30 mm, 10 um); Mobile phase: 0.1% NH3H2O ETOH; from 35% to 35%; Flow rate: 70 mL/min) to give (5R,6R)-5-(4-(4-(dimethoxymethyl)piperidin-1-yl)phenyl)-6-isopropyl-5,6,7,8-tetrahydronaphthalen-2-ol (130 mg, 307 μmol, 32.5%) as white solid. LC-MS (ESI⁺) m/z: 424.3 (M+H)⁺.

Step 4: To a solution of (5R,6R)-5-(4-(4-(dimethoxymethyl)piperidin-1-yl)phenyl)-6-isopropyl-5,6,7,8-tetrahydronaphthalen-2-ol (140 mg, 1 eq., 331 μmol) in DCM (4 mL) was added TFA (113 mg, 76.4 μL, 3 eq., 992 μmol). The mixture was stirred at 20° C. for 4 hour. The mixture was concentrated in vacuo to give crude product. No further purification as it is used for the next step directly.

LC-MS (ESI+) m/z: 378.2 (M+H)+

Step 5: To a solution of 1-(4-((1R,2R)-6-hydroxy-2-isopropyl-1,2,3,4-tetrahydronaphthalen-1-yl)phenyl)piperidine-4-carbaldehyde (100 mg, 1 eq., 265 μmol) and (R)-3-(1-oxo-5-(piperazin-1-yl)isoindolin-2-yl)piperidine-2,6-dione (113 mg, 1.3 eq., 344 μmol) in DCE (5 mL) was added dropwise 4-methylmorpholine (26.8 mg, 1 eq., 265 μmol) to pH=8 at 20° C. After addition, the mixture was stirred at this temperature for 0.5 hour, and then sodium triacetoxyhydroborate (140 mg, 2.5 eq., 662 μmol) was added dropwise at 20° C. The resulting mixture was stirred at 20° C. for 2 hour. The crude was purified by prep HPLC together (Welch Xtimate C18 150*25 mm*5 um, water (NH₃H₂O+NH₄HCO₃)-ACN as a mobile phase, from 35% to 65%, Gradient Time (min): 11, Flow Rate (ml/min): 25) to give (R)-3-(5-(4-((1-(4-((1R,2R)-6-hydroxy-2-isopropyl-1,2,3,4-tetrahydronaphthalen-1-yl)phenyl)piperidin-4-yl)methyl)piperazin-1-yl)-1-oxoisoindolin-2-yl)piperidine-2,6-dione (70.5 mg, 102 μmol, 38.6%) as a white solid.

LC-MS (ESI+) m/z: 690.3 (M+H),+

¹H NMR (500 MHz, METHANOL-d4) δ: ppm: 8.34 (s, 1H), 7.67 (d, J=8.54 Hz, 1H), 7.09-7.15 (m, 2H), 6.92-6.96 (m, 2H), 6.86-6.91 (m, 2H), 6.68 (d, J=8.39 Hz, 1H), 6.58 (d, J=2.29 Hz, 1H), 6.47 (dd, J=8.32, 2.52 Hz, 1H), 5.12 (dd, J=13.35, 5.11 Hz, 1H), 4.34-4.50 (m, 2H), 4.14 (d, J=3.81 Hz, 1H), 3.63 (br d, J=11.44 Hz, 2H), 3.46 (br s, 4H), 2.89-2.98 (m, 2H), 2.83-2.88 (m, 4H), 2.76-2.81 (m, 1H), 2.69 (brt, J=12.13 Hz, 2H), 2.52-2.59 (m, 2H), 2.48 (dd, J=13.20, 4.65 Hz, 1H), 2.12-2.21 (m, 1H), 1.92 (br d, J=12.97 Hz, 2H), 1.75-1.86 (m, 2H), 1.57-1.68 (m, 2H), 1.36-1.46 (m, 3H), 1.09 (d, J=6.56 Hz, 3H), 0.66 (d, J=6.71 Hz, 3H).

### Example 215. Preparation of (I-112) (S)-3-(5-(4-((1-(4-((1S,2R)-2-ethyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

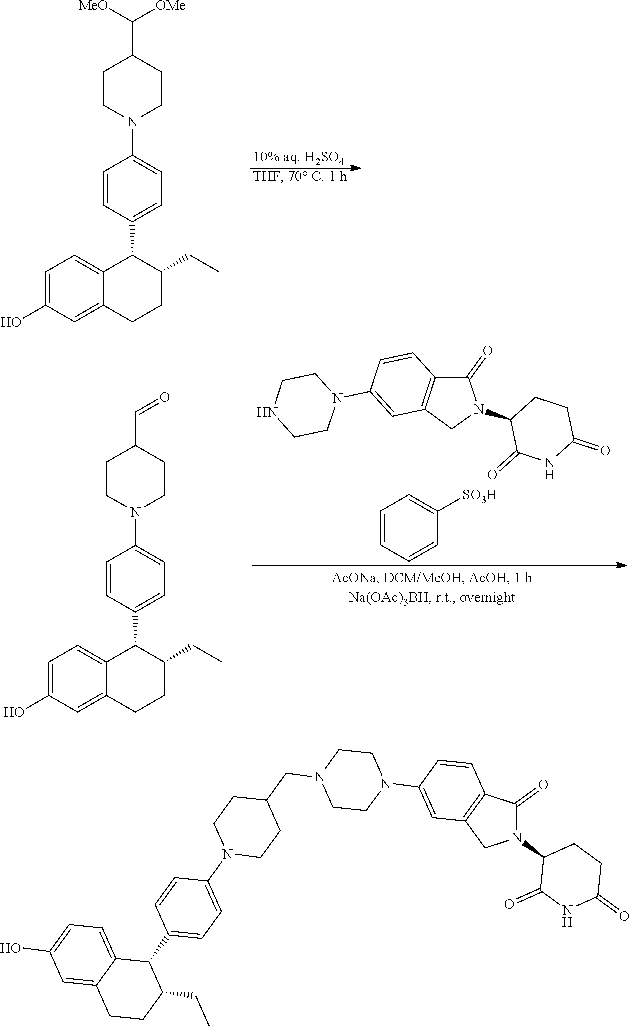

Step 1: To a solution of (5S,6R)-5-(4-(4-(dimethoxymethyl)piperidin-1-yl)phenyl)-6-ethyl-5,6,7,8-tetrahydronaphthalen-2-ol (40 mg, 95 μmol) in THF (1 mL) and 10% H₂SO₄ (1 mL) was stirred at 70° C. for 1 h. The reaction mixture was quenched by addition 20 mL NaHCO₃ at 0° C., then extracted with ethyl acetate (20 mL×2). The combined organic layers were washed with 20 mL brine dried over Na₂SO₄, filtered and concentrated under reduced pressure to give 1-(4-((1R,2S)-2-ethyl-6-hydroxy-1,2,3,4-tetrahydronaphthalen-1-yl)phenyl)piperidine-4-carbaldehyde (35 mg, 98% Yield) as a yellow solid. LC-MS (ESI+) m/z: 364.3 (M+H)⁺.

Step 2: A mixture of 1-(4-((1S,2R)-2-ethyl-6-hydroxy-1,2,3,4-tetrahydronaphthalen-1-yl)phenyl)piperidine-4-carbaldehyde (35 mg, 93 μmol), (S)-3-(1-oxo-5-(piperazin-1-yl)isoindolin-2-yl)piperidine-2,6-dione benzenesulfonate (68 mg, 0.14 mmol) and Sodium acetate (38 mg, 0.46 mmol) and Acetic acid (11 mg, 0.19 mmol) in THF (1 mL) and MeOH (1 mL) at 25° C. for 1 h, then Sodium triacetoxyborohydride (39 mg, 0.19 mmol) was added to the mixture and was stirred at 25° C. for 16 h. The mixture was added H₂O (20 mL), extracted with ethyl acetate (20 mL×2). The combined extracts was dried over anhydrous Na₂SO₄, filtered and concentrated to dryness in vacuo to give a residue. The residue was purified by prep-HPLC (column: O-Welch C18 150*30 mm*5 um; mobile phase: [water(FA)-ACN]; B % 10%-50%, 8 min) to give (S)-3-(5-(4-((1-(4-((1S,2R)-2-ethyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (31.3 mg, 50% Yield) as a white solid. LC-MS (ESI+) m/z: 676.2 (M+H)+.

LCMS: calc. for C₄₁H₄₉N₅O₄:675.87, found: [M+H]⁺ 676.2.

HPLC: 100% purity at 220 nm.

¹HNMR (400 MHz, DMSO-d₆) δ=10.96 (s, 1H), 9.03 (br s, 1H), 8.15 (s, 1H), 7.52 (d, J=8.3 Hz, 1H), 7.11-7.02 (m, 2H), 6.77 (s, 4H), 6.62 (d. J=8.5 Hz, 1H), 6.51 (s, 1H), 6.43 (dd, J=2.2, 8.3 Hz, 1H), 5.05 (dd, J=5.0, 13.4 Hz, 1H), 4.38-4.29 (m, 1H), 4.25-4.17 (m, 1H), 3.93 (br d, J=4.9 Hz, 1H), 3.59 (br d, J=11.6 Hz, 2H), 3.29 (br s, 5H), 2.96-2.72 (m, 4H), 2.59 (br d, J=10.4 Hz, 4H), 2.44-2.34 (m, 2H), 2.22 (br d, J=6.8 Hz, 2H), 2.00-1.93 (m, 1H), 1.80 (br d, J=11.4 Hz, 2H), 1.73-1.56 (m, 3H), 1.54-1.43 (m, 1H), 1.26-1.14 (m, 3H), 0.91 (t, J=7.2 Hz, 3H), 0.77 (td, J=7.3, 14.3 Hz, 1H)

SFC: retention time, 2.691 min; Arca, 93.989%; Stereochemistry was arbitrarily assigned.

### Example 216. Preparation of (I-111) (S)-3-(5-(4-((1-(4-((1R,2S)-2-ethyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

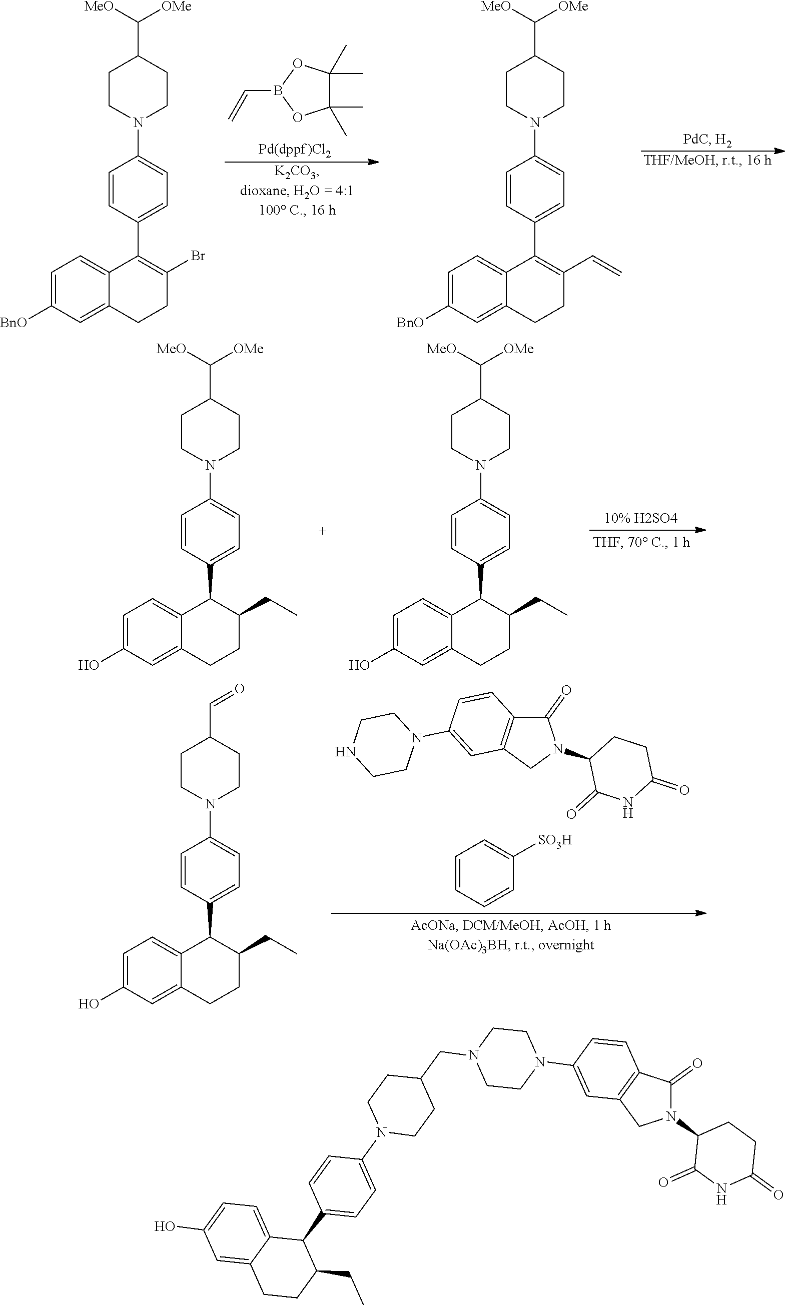

Step 1: To a mixture of [1,1′-Bis(diphenylphosphino)ferrocene]dichloropalladium(II) Complex (74.44 mg, 91.15 μmol) was added to the mixture of 1-(4-(6-(benzyloxy)-2-bromo-3,4-dihydronaphthalen-1-yl)phenyl)-4-(dimethoxymethyl)piperidine (500 mg, 911.5 μmol), Vinylboronic Acid Pinacol Ester (168.5 mg, 186 μL, 1.094 mmol) and K₂CO₃ (377.9 mg, 2.735 mmol) in Dioxane (8 mL) and H₂O (2 mL) at r.t. N₂ was bubbled into the mixture for 5 min. The reaction mixture was heated at 100° C. for 16 h. The reaction mixture was added 100 mL H₂O and extracted with ethyl acetate (100 mL×2). The combined organic layers was dried over Na₂SO₄, filtered and concentrated under reduced pressure to give a residue. The residue was purified by flash silica gel chromatography (ethyl acetate in petroleum ether=0% to 30%) to give the product of 1-(4-(6-(benzyloxy)-2-vinyl-3,4-dihydronaphthalen-1-yl)phenyl)-4-(dimethoxymethyl)piperidine (230 mg, 36.3% Yield) as a yellow solid. LC-MS (ESI⁺) m/z: 496.1 (M+H)⁺.

Step 2: A mixture of 1-(4-(6-(benzyloxy)-2-vinyl-3,4-dihydronaphthalen-1-yl)phenyl)-4-(dimethoxymethyl)piperidine (230 mg, 331 μmol), dihydrogen (669 μg, 331 μmol), Pd/C (230 mg, 10% Wt, 216 μmol) in MeOH (5 mL) was degassed and purged with H₂ for 3 times, and then the mixture was stirred at 25° C. for 16 h under H₂ atmosphere (15 psi). The reaction mixture was filtered and concentrated under reduced pressure to give a residue. The residue was further separated by SFC (condition: column: DAICEL CHIRALPAK IG (250 mm*30 mm, 10 um); mobile phase: [0.1% NH3H2O-MEOH]; B %: 60%-60%, min) to give (5R,6S)-5-(4-(4-(dimethoxymethyl)piperidin-1-yl)phenyl)-6-ethyl-5,6,7,8-tetrahydronaphthalen-2-ol (40 mg, 27% Yield) was obtained as a white solid. LC-MS (ESI+) m/z: 410.1 (M+H)⁺ and (5S,6R)-5-(4-(4-(dimethoxymethyl)piperidin-1-yl)phenyl)-6-ethyl-5,6,7,8-tetrahydronaphthalen-2-ol (40 mg, 29% Yield) was obtained as a white solid. LC-MS (ESI⁺) m/z: 410.2 (M+H)⁺.

Step 3: To a solution of (5R,6S)-5-(4-(4-(dimethoxymethyl)piperidin-1-yl)phenyl)-6-ethyl-5,6,7,8-tetrahydronaphthalen-2-ol (40 mg, 89 μmol) in THF (1 mL) and 10% H₂SO₄ (1 mL) was stirred at 70° C. for 1 h. The reaction mixture was quenched by addition 20 mL NaHCO₃ at 0° C., then extracted with ethyl acetate (20 mL×2). The combined organic layers were washed with 20 mL brine dried over Na₂SO₄, filtered and concentrated under reduced pressure to give 1-(4-((1R,2S)-2-ethyl-6-hydroxy-1,2,3,4-tetrahydronaphthalen-1-yl)phenyl)piperidine-4-carbaldehyde (35 mg, 100% Yield) as a yellow solid. LC-MS (ESI⁺) m/z: 364.3 (M+H)⁺.

Step 4: A mixture of 1-(4-((1R,2S)-2-ethyl-6-hydroxy-1,2,3,4-tetrahydronaphthalen-1-yl)phenyl)piperidine-4-carbaldehyde (35 mg, 89 μmol), (S)-3-(1-oxo-5-(piperazin-1-yl)isoindolin-2-yl)piperidine-2,6-dione benzenesulfonate (65 mg, 0.13 mmol) and Sodium acetate (37 mg, 0.45 mmol) and Acetic acid (11 mg, 10 μL, 0.18 mmol) in THF (1 mL) and MeOH (1 mL) at 25° C. for 1 h, then Sodium triacetoxyborohydride (38 mg, 0.18 mmol) was added to the mixture and was stirred at 25° C. for 16 h. The mixture was added H₂O (20 mL), extracted with ethyl acetate (20 mL×2). The combined extracts was dried over anhydrous Na₂SO₄, filtered and concentrated to dryness in vacuo to give a residue. The residue was purified by prep-HPLC (column: O-Welch Xtimate C18 150*30 mm*5 um; mobile phase: [water(FA)-ACN]; B % 15%-55%, 8 min) to give (S)-3-(5-(4-((1-(4-((1R,2S)-2-ethyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (30.6 mg, 51% Yield) as a white solid. LC-MS (ESI⁺) m/z: 676.2 (M+H)⁺

LCMS: calc. for C₄₁H₄₉N₅O₄:675.87, found: [M+H]⁺ 676.2.

HPLC: 100% purity at 220 nm.

¹HNMR (400 MHz, DMSO-d₆) δ=10.96 (s, 1H), 9.03 (s, 1H), 8.14 (s, 1H), 7.52 (d, J=8.6 Hz, 1H), 7.10-7.03 (m, 2H), 6.77 (s, 4H), 6.62 (d, J=8.5 Hz, 1H), 6.51 (s, 1H), 6.43 (dd, J=2.4, 8.2 Hz, 1H), 5.05 (dd, J=5.0, 13.2 Hz, 1H), 4.38-4.29 (m, 1H), 4.25-4.17 (m, 1H), 3.93 (d, J=5.0 Hz, 1H), 3.60 (br d, J=12.5 Hz, 2H), 3.29 (br s, 5H), 2.96-2.73 (m, 3H), 2.59 (br d, J=12.2 Hz, 5H), 2.38 (br d, J=4.6 Hz, 2H), 2.22 (br d, J=6.7 Hz, 2H), 2.00-1.92 (m, 1H), 1.80 (br d, J=11.6 Hz, 2H), 1.73-1.56 (m, 3H), 1.49 (dt, J=6.2, 12.3 Hz, 1H), 1.26-1.14 (m, 3H), 0.91 (t, J=7.2 Hz, 3H), 0.78 (qd, J=6.9, 13.7 Hz, 1H)

SFC: retention time, 2.656 min; Area, 93.493%; Stereochemistry was arbitrarily assigned

### Example 217. Preparation of (I-124) (S)-3-(5-(4-((1-(4-((1R,2S)-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione and (I-125) (S)-3-(5-(4-((1-(4-((1S,2R)-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione

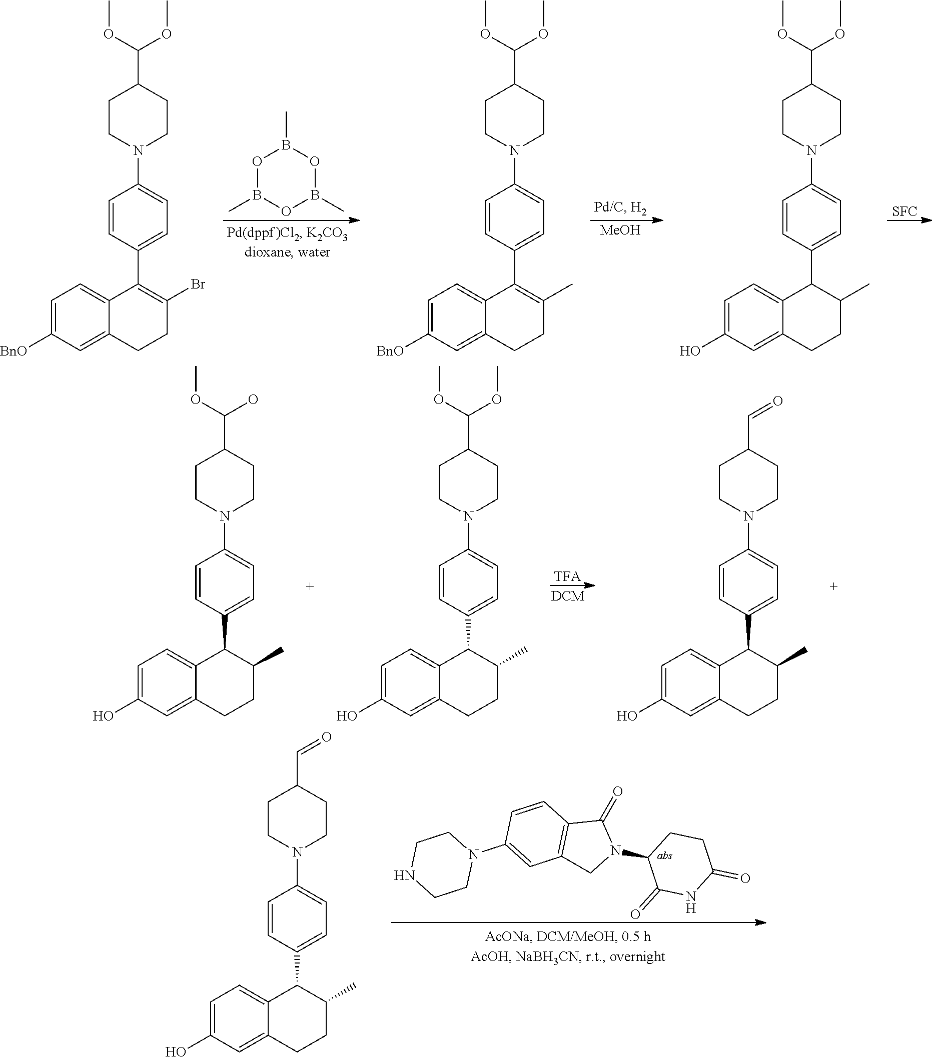

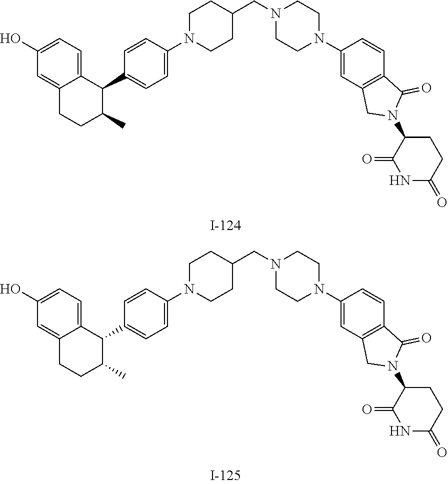 [SMILES: C[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; C[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1]

Step 1: A mixture of 1-(4-(6-(benzyloxy)-2-bromo-3,4-dihydronaphthalen-1-yl)phenyl)-4-(dimethoxymethyl)piperidine (1.0 g, 1.82 mmol, 1 eq), 2,4,6-trimethyl-1,3,5,2,4,6-trioxatriborinane (457.73 mg, 1.82 mmol, 509.72 uL, 50% purity, 1 eq), Pd(dppf)Cl₂ (133.40 mg, 182.31 μmol, 0.1 eq) and K₂CO₃ (503.93 mg, 3.65 mmol, 2 eq) in dioxane (15 mL) and H₂O (4 mL) was degassed and purged with N₂ for 3 times, and then the mixture was stirred at 80° C. for 16 hr under N₂ atmosphere. The mixture was concentrated in reduced pressure. The residue was poured into water (20 mL). The aqueous phase was extracted with ethyl acetate (40 mL*3). The combined organic phase was washed with brine (20 mL), dried with Na₂SO₄, filtered and concentrated in vacuum. The residue was purified by silica gel chromatography (Petroleum ether/Ethyl acetate=3/1). 1-(4-(6-(benzyloxy)-2-methyl-3,4-dihydronaphthalen-1-yl)phenyl)-4-(dimethoxymethyl)piperidine (450 mg, 930.44 μmol, 51.04% yield) was obtained as oil.

LC-MS (ESI+) m/z: 484.3 (M+H)+.

Step 2: A mixture of 1-(4-(6-(benzyloxy)-2-methyl-3,4-dihydronaphthalen-1-yl)phenyl)-4-(dimethoxymethyl)piperidine (280 mg, 578.94 μmol, 1 eq) and Pd/C (123.22 mg, 115.79 μmol, 10% purity, 0.2 eq) in MeOH (3 mL) was degassed and purged with H₂ for 3 times, and then the mixture was stirred at 25° C. for 2 hr under H₂ atmosphere. LCMS showed that the starting material was consumed and the desired product was detected. The reaction mixture was filtered under reduced pressure to give 5-(4-(4-(dimethoxymethyl)piperidin-1-yl)phenyl)-6-methyl-5,6,7,8-tetrahydronaphthalen-2-ol. Then 5-(4-(4-(dimethoxymethyl)piperidin-1-yl)phenyl)-6-methyl-5,6,7,8-tetrahydronaphthalen-2-ol (200 mg, 505.65 μmol, 87.34% yield) was obtained as a white solid.

LC-MS (ESI+) m/z: 396.2 (M+H)+.

Step 3: 5-(4-(4-(dimethoxymethyl)piperidin-1-yl)phenyl)-6-methyl-5,6,7,8-tetrahydronaphthalen-2-ol (500 mg) was separated by chiral SFC(Column DAICEL CHIRALPAK AS (250 mm*30 mm, 10 um) Condition 0.1% NH₃H₂O EtOH Begin B 55% End B 55%), 0.1% NH₃H₂O EtOH) (5R,6S)-5-(4-(4-(dimethoxymethyl)piperidin-1-yl)phenyl)-6-methyl-5,6,7,8-tetrahydronaphthalen-2-ol (200 mg), (5S,6R)-5-(4-(4-(dimethoxymethyl)piperidin-1-yl)phenyl)-6-methyl-5,6,7,8-tetrahydronaphthalen-2-ol (200 mg).

LC-MS (ESI+) m/z: 396.0 (M+H)+.

Step 4: 1-(4-(1R,2S)-6-hydroxy-2-methyl-1,2,3,4-tetrahydronaphthalen-1-yl)phenyl)piperidine-4-carbaldehyde: To a solution of (5R,6S)-5-(4-(4-(dimethoxymethyl)piperidin-1-yl)phenyl)-6-methyl-5,6,7,8-tetrahydronaphthalen-2-ol (100 mg, 252.82 μmol, 1 eq) in DCM (8 mL) was added TFA (28.83 mg, 252.82 μmol, 18.72 uL, 1 eq). The mixture was stirred at 25° C. for 2 hr. LCMS showed that the starting material was consumed and the desired product was detected. The reaction mixture was filtered under reduced pressure to give a residue. 1-(4-((1R,2S)-6-hydroxy-2-methyl-1,2,3,4-tetrahydronaphthalen-1-yl)phenyl)piperidine-4-carbaldehyde (70 mg, 200.31 μmol, 79.23% yield) was obtained as oil.

1-(4-((1S,2R)-6-hydroxy-2-methyl-1,2,3,4-tetrahydronaphthalen-1-yl)phenyl)piperidine-4-carbaldehyde: To a solution of (5S,6R)-5-(4-(4-(dimethoxymethyl)piperidin-1-yl)phenyl)-6-methyl-5,6,7,8-tetrahydronaphthalen-2-ol (150 mg, 379.24 μmol, 1 eq) in DCM (8 mL) (150 mg, 379.24 μmol, 1 eq) in DCM (8 mL) was added TFA (43.24 mg, 379.24 μmol, 28.08 uL, 1 eq). The mixture was stirred at 25° C. for 2 hr. LCMS showed that the starting material was consumed and the desired product was detected. The reaction mixture was filtered under reduced pressure to give a residue. 1-(4-((1S,2R)-6-hydroxy-2-methyl-1,2,3,4-tetrahydronaphthalen-1-yl)phenyl)piperidine-4-carbaldehyde (100 mg, 286.15 μmol, 75.45% yield) was obtained as oil.

LC-MS (ESI+) m/z: 368.2 (M+H)+.

Step 5: To a solution of 1-(4-((1R,2S)-6-hydroxy-2-methyl-1,2,3,4-tetrahydronaphthalen-1-yl)phenyl)piperidine-4-carbaldehyde (126.68 mg, 362.50 μmol, 1 eq) and (S)-3-(1-oxo-5-(piperazin-1-yl)isoindolin-2-yl)piperidine-2,6-dione (119.03 mg, 362.50 μmol, 1 eq) in MeOH (2 mL) and DCM (3 mL) was added NMM (3.67 mg, 36.25 μmol, 3.99 uL, 0.1 eq) and C6H10BO6Na (153.66 mg, 724.99 μmol, 2 eq). The mixture was stirred at 25° C. for 2 hr. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water(FA)-ACN]; B %: 10%-40%, 10 min) to give (S)-3-(5-(4-((1-(4-((1R,2S)-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione (50 mg, 75.55 μmol, 20.84% yield) was obtained as white solid.

LC-MS (ESI+) m/z: 662.5 (M+H)+.

HPLC: 100% purity at 220 nm.

¹H NMR (400 MHZ, METHANOL-d4) δ=7.63 (d, J=8.9 Hz, 1H), 7.11-7.06 (m, 2H), 6.87-6.80 (m, 4H), 6.63 (d, J=8.3 Hz, 1H), 6.54 (d, J=2.0 Hz, 1H), 6.49-6.39 (m, 1H), 5.10-5.05 (m, 1H), 4.43-4.35 (m, 2H), 3.89 (d, J=5.1 Hz, 1H), 3.60 (br d, J=11.4 Hz, 2H), 3.44 (br s, 4H), 2.91-2.75 (m, 8H), 2.70-2.56 (m, 4H), 2.49-2.39 (m, 1H), 2.14-2.00 (m, 2H), 1.88 (br d, J=12.6 Hz, 2H), 1.85-1.78 (m, 1H), 1.61 (br s, 1H), 1.53-1.47 (m, 1H), 1.35 (br s, 2H), 0.73 (d, J=6.8 Hz, 3H).

Step 6: To a solution of 1-(4-((1S,2R)-6-hydroxy-2-methyl-1,2,3,4-tetrahydronaphthalen-]-yl)phenyl)piperidine-4-carbaldehyde (180 mg, 515.07 umol, 1 eq) and (S)-3-(1-oxo-5-(piperazin-1-yl)isoindolin-2-yl)piperidine-2,6-dione (169.13 mg, 515.07 umol, 1 eq) in DCE (3 mL) and MeOH (2 mL) was added NMM (5.21 mg, 51.51 umol, 5.66 uL, 0.1 eq) and C6H10BO6Na (218.33 mg, 1.03 mmol, 2 eq). The mixture was stirred at 25° C. for 2 hr. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water(FA)-ACN]; B %: 10%-40%, 10 min) to give (S)-3-(5-(4-((1-(4-((1S,2R)-6-hydroxy-2-methyl-1,2,3,4-tetrahydronaphthalen-1-yl)phenyl)piperidin-4-yl)methyl)piperazin-1-yl)-1-oxoisoindolin-2-yl)piperidine-2,6-dione (34 mg, 51.37 μmol, 9.97% yield) was obtained as a white solid.

LC-MS (ESI+) m/z: 662.5 (M+H)⁺.

HPLC: 100% purity at 220 nm.

¹H NMR (400 MHz, METHANOL-d4) δ=8.43-8.34 (m, 1H), 7.65 (br d, J=8.5 Hz, 1H), 7.16-7.06 (m, 2H), 6.90-6.82 (m, 4H), 6.67 (br d, J=8.1 Hz, 1H), 6.57 (br s, 1H), 6.47 (br d, J=7.9 Hz, 1H), 5.13-5.07 (m, 1H), 4.41 (br d, J=6.1 Hz, 2H), 3.94-3.89 (m, 1H), 3.62 (br d, J=11.9 Hz, 2H), 3.43 (br s, 4H), 2.92-2.76 (m, 8H), 2.72-2.65 (m, 2H), 2.54-2.43 (m, 3H), 2.17-2.03 (m, 2H), 1.91 (br d, J=12.4 Hz, 2H), 1.84-1.75 (m, 1H), 1.62-1.47 (m, 2H), 1.47-1.36 (m, 2H), 0.77 (br d, J=6.8 Hz, 3H).

### Example 218. Preparation of (I-103) (S)-3-(5-(4-((1-(4-((S)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

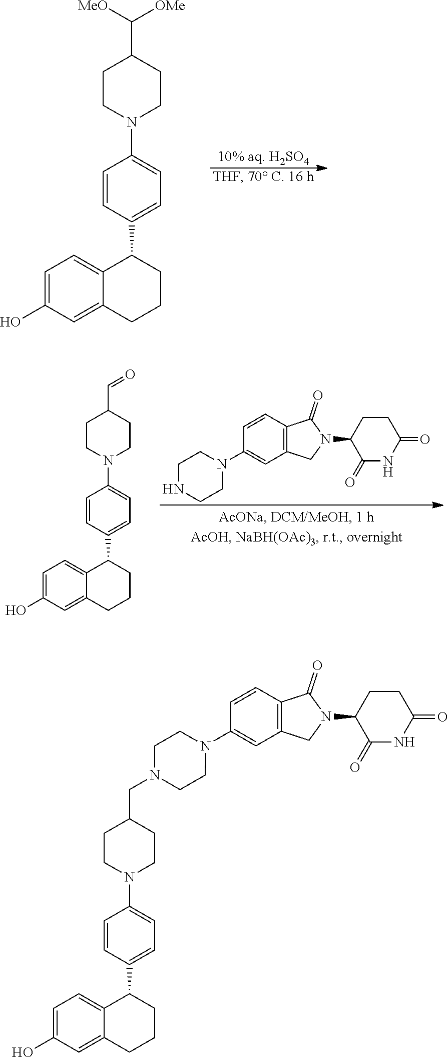 [SMILES: COC(OC)C1CCN(c2ccc([C@@H]3CCCc4cc(O)ccc43)cc2)CC1 ; "THF, 70° C. 16 h" ; "10% aq. H2SO4" ; O=CC1CCN(c2ccc([C@@H]3CCCc4cc(O)ccc43)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7CCCc8cc(O)ccc87)cc6)CC5)CC4)ccc3C2=O)C(=O)N1]

Step 1: To a solution of (S)-5-(4-(4-(dimethoxymethyl)piperidin-1-yl)phenyl)-5,6,7,8-tetrahydronaphthalen-2-ol (59 mg, 1 Eq, 0.15 mmol) in THF (10 mL) was added sulfuric acid (15 mg, 10 mL, 1 Eq, 0.15 mmol). The mixture was stirred at 70° C. for 4 hour. LCMS showed 97.08% desired MS. The mixture was adjusted to pH 8-9 by progressively adding NaHCO₃. The reaction mixture was quenched by addition 20 mL EtOAc at 20° C., and then diluted with 20 mL H₂O and extracted with 50 mL (25 mL*2) EtOAc. The combined organic layers were washed with 10 mL (10 mL*1), dried over Na₂SO₄. The reaction was filtered and concentrated under reduced pressure to give (S)-1-(4-(6-hydroxy-1,2,3,4-tetrahydronaphthalen-1-yl)phenyl)piperidine-4-carbaldehyde (70 mg, 0.21 mmol, 130%, 100% Purity) was obtained as a colorless oil. LC-MS (ESI⁺) m/z: 354.2 (M+H)⁺.

Step 2: A mixture of (S)-1-(4-(6-hydroxy-1,2,3,4-tetrahydronaphthalen-1-yl)phenyl)piperidine-4-carbaldehyde (70 mg, 1 Eq, 0.21 mmol), (S)-3-(1-oxo-5-(piperazin-1-yl)isoindolin-2-yl)piperidine-2,6-dione (69 mg, 1 Eq, 0.21 mmol) and Sodium acetate (86 mg, 5 Eq, 1.0 mmol) in DCM (1 mL) and MeOH (1 mL) at 20° C. for 0.5 hour, pH=8, then acetic acid (38 mg, 3 Eq, 0.63 mmol) at 20° C. for 1 hour, pH=6, then added Sodium triacetoxyborohydride (88 mg, 2 Eq, 0.42 mmol) the mixture, and then the mixture was stirred at 25° C. for 16 hour. LCMS showed 86.85% desired MS. The reaction was concentrated to give (S)-3-(5-(4-((1-(4-((S)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione as a light yellow oil. The light yellow oil was purified by preparative high-performance liquid chromatography. Condition: Column: O-Welch C18 150*30 mm*5 um; A: water(FA); B: CAN; at the beginning: A (100%) and B (0%); at the end: A: (60%) and B (40%); Gradient Time (min) 10; 100% B hold Time (min) 2, Flow Rate (ml/min) 30. LCMS showed 100% desired MS. The pure fractions were collected and the solvent was evaporated under vacuum. The aqueous layer was lyophilized to dryness to give (S)-3-(5-(4-((1-(4-((S)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (28.3 mg, 43.7 μmol, 21%, 100% Purity) as a white solid. LC-MS (ESI+) m/z: 648.2 (M+H)⁺.

LCMS: calc. for C₃₉H₄₅N₅O₄:647.35, found: [M+H]⁺ 648.2.

HPLC: 100% purity at 220 nm.

¹H NMR (400 MHz, DMSO-d₆) δ=10.99-10.88 (m, 1H), 9.16-8.97 (m, 1H), 8.20-8.14 (m, 0.481H), 7.55-7.48 (m, 1H), 7.09-7.03 (m, 2H), 6.91-6.82 (m, 4H), 6.54-6.47 (m, 2H), 6.46-6.40 (m, 1H), 5.15-4.97 (m, 1H), 4.37-4.30 (m, 1H), 4.25-4.15 (m, 1H), 3.91-3.82 (m, 1H), 3.65-3.58 (m, 2H), 3.31-3.29 (m, 6H), 2.96-2.84 (m, 1H), 2.73-2.59 (m, 4H), 2.39 (br s, 3H), 2.26-2.20 (m, 2H), 2.00-1.93 (m, 2H), 1.90-1.56 (m, 7H), 1.29-1.19 (m, 2H)

SFC: retention time, 2.759 min; Area, 92.821%; Stereochemistry was arbitrarily assigned

### Example 219. Preparation of (I-102) (S)-3-(5-(4-((1-(4-((R)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

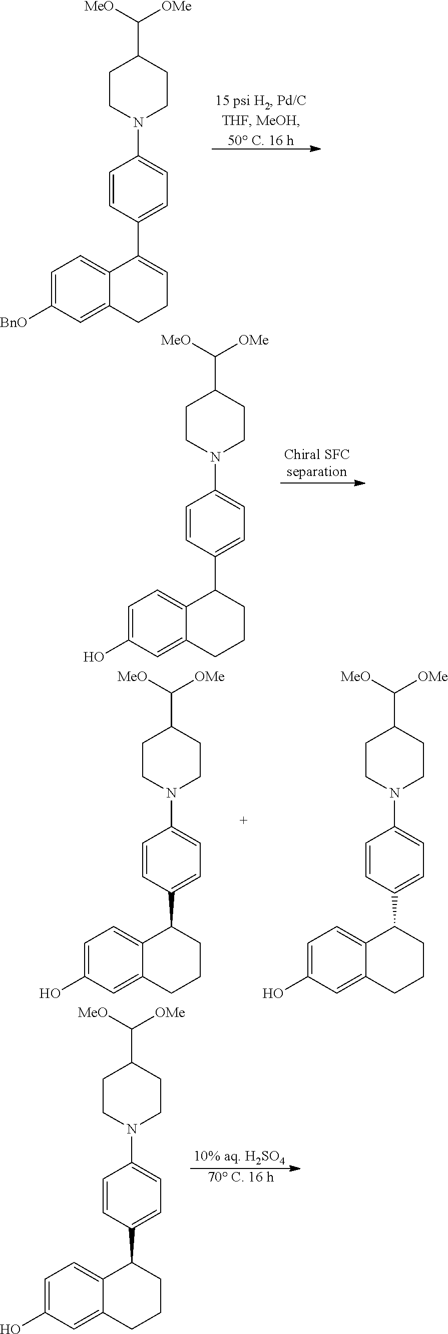

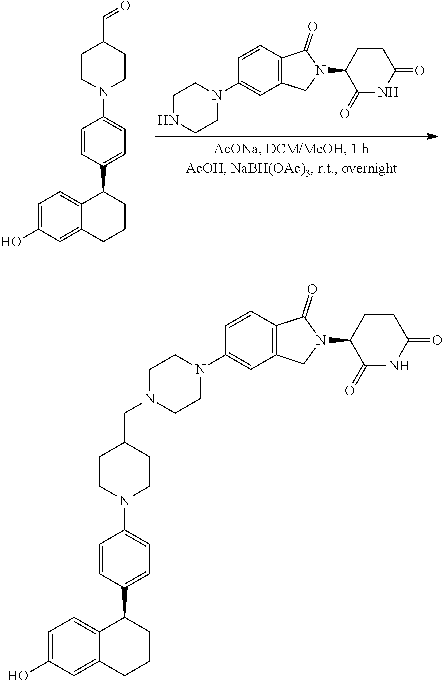 [SMILES: O=CC1CCN(c2ccc([C@H]3CCCc4cc(O)ccc43)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7CCCc8cc(O)ccc87)cc6)CC5)CC4)ccc3C2=O)C(=O)N1]

Step 1: A mixture of 1-(4-(6-(benzyloxy)-3,4-dihydronaphthalen-1-yl)phenyl)-4-(dimethoxymethyl)piperidine (400 mg, 1 Eq, 852 μmol), Pd/C (453 mg, 5 Eq, 4.26 mmol) in MeOH (6 mL) and THF (6 mL) was degassed and purged with H₂ for 3 times, and then the mixture was stirred at 50° C. for 16 hour under H₂ atmosphere (15 psi). LCMS showed 78% desired. TLC (petroleum ether:ethyl acetate=3:1, UV, R_{f}=0.5) showed one main new spot was observed. The reaction was filtered and concentrated to dryness in vacuo to give a blue solid. The blue solid was subjected to column chromatography over silica gel (gradient elution: 0-100% EtOAc). LCMS showed 83% desired. The desired fractions were collected, and concentrated to dryness in vacuo to give 5-(4-(4-(dimethoxymethyl)piperidin-1-yl)phenyl)-5,6,7,8-tetrahydronaphthalen-2-ol (275 mg, 0.60 mmol, 70%, 83% Purity) as a white solid. LC-MS (ESI⁺) m/z: 382.2 (M+H)⁺.

Step 2: The white solid was purified by SFC. Column: DAICEL CHIRALCEL OJ (250 mm*30 mm, 10 um); Condition: 0.1% NH₃H₂O ETOH; At the beginning: A (55%) and B (45%); At the end: A: (55%) and B (45%); Flow Rate (ml/min) δ0. The aqueous phase was lyophilized to dryness to give (R)-5-(4-(4-(dimethoxymethyl)piperidin-1-yl)phenyl)-5,6,7,8-tetrahydronaphthalen-2-ol (57 mg, 0.14 mmol, 19%, 91.19% Purity) as a yellow solid and (S)-5-(4-(4-(dimethoxymethyl)piperidin-1-yl)phenyl)-5,6,7,8-tetrahydronaphthalen-2-ol (59 mg, 0.14 mmol, 20%, 91.52% Purity) as a colorless oil. LC-MS (ESI⁺) m/z: 382.1 (M+H)⁺.

Step 3: To a solution of (R)-5-(4-(4-(dimethoxymethyl)piperidin-1-yl)phenyl)-5,6,7,8-tetrahydronaphthalen-2-ol (52 mg, 1 Eq, 0.14 mmol) in THF (15 mL) was added 10% sulfuric acid (13 mg, 15 mL, 1 Eq, 0.14 mmol). The mixture was stirred at 70° C. for 4 hour. LCMS showed 97.88% desired MS. The mixture was adjusted to Ph=8-9 by progressively adding NaHCO₃. The reaction mixture was quenched by addition 20 mL EtOAc at 20° C., and then diluted with 20 mL H₂O and extracted with 50 mL (25 mL*2) EtOAc. The combined organic layers were washed with 10 mL, dried over Na₂SO₄. The reaction was filtered and concentrated under reduced pressure to give (R)-1-(4-(6-hydroxy-1,2,3,4-tetrahydronaphthalen-1-yl)phenyl)piperidine-4-carbaldehyde (49 mg, 0.14 mmol, 100%, 97.87% Purity) was obtained as a colorless oil. LC-MS (ESI⁺) m/z: 354.2 (M+H)⁺

Step 4: A mixture of (R)-1-(4-(6-hydroxy-1,2,3,4-tetrahydronaphthalen-1-yl)phenyl)piperidine-4-carbaldehyde (49 mg, 1 Eq, 0.15 mmol), (S)-3-(1-oxo-5-(piperazin-1-yl)isoindolin-2-yl)piperidine-2,6-dione (48 mg, 1 Eq, 0.15 mmol) and sodium acetate (60 mg, 5 Eq, 0.73 mmol) in DCM (1 mL) and MeOH (1 mL) at 20° C. for 0.5 hour, pH=8, then acetic acid (26 mg, 25 μL, 3 Eq, 0.44 mmol) at 20° C. for 1 hour, pH=6, then added sodium triacetoxyhydroborate (62 mg, 2 Eq. 0.29 mmol) the mixture, and then the mixture was stirred at 25° C. for 16 hour. LCMS showed 83.9% desired MS. The reaction was concentrated to give (S)-3-(5-(4-((1-(4-((R)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione as a light yellow oil. The light yellow oil was purified by preparative high-performance liquid chromatography. Condition: Column: O-Welch C18 150*30 mm*5 um; A: water(FA); B: CAN; At the beginning: A (100%) and B (0%); At the end: A: (60%) and B (40%); Gradient Time (min) 10; 100% B hold Time (min) 2, Flow Rate (ml/min) 30. LCMS showed 98.30% desired MS. The pure fractions were collected and the solvent was evaporated under vacuum. The aqueous layer was lyophilized to dryness to give (S)-3-(5-(4-((1-(4-((R)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (21.7 mg, 33.5 μmol, 23%, 100% Purity) as a white solid. LC-MS (ESI+) m/z: 648.2 (M+H)⁺.

LCMS: calc. for C₃₉H₄₅N₅O₄:647.35, found: [M+H]⁺ 648.2.

HPLC: 100% purity at 220 nm.

¹H NMR (400 MHZ, DMSO-d₆) δ=10.98-10.91 (m, 1H), 9.08-9.00 (m, 1H), 8.18-8.14 (m, 1H), 7.55-7.48 (m, 1H), 7.09-7.03 (m, 2H), 6.89-6.80 (m, 4H), 6.53-6.47 (m, 2H), 6.45-6.40 (m, 1H), 5.10-4.99 (m, 1H), 4.38-4.28 (m, 1H), 4.25-4.15 (m, 1H), 3.89-3.84 (m, 1H), 3.65-3.56 (m, 3H), 2.93-2.85 (m, 1H), 2.79-2.53 (m, 8H), 2.33 (br s, 4H), 2.24-2.20 (m, 2H), 2.00-1.92 (m, 2H), 1.80 (br d, J=11.2 Hz, 7H), 1.26-1.18 (m, 2H)

SFC: retention time, 2.354 min; Area, 93.433%; Stereochemistry was arbitrarily assigned

### Example 220. Preparation of (I-119) (S)-3-(5-(4-((1-(4-((1S,2R)-2-cyclohexyl-6-hydroxy-1,2,3,4-tetrahydronaphthalen-1-yl)-3,5-difluorophenyl)piperidin-4-yl)methyl)piperazin-1-yl)-1-oxoisoindolin-2-yl)piperidine-2,6-dione

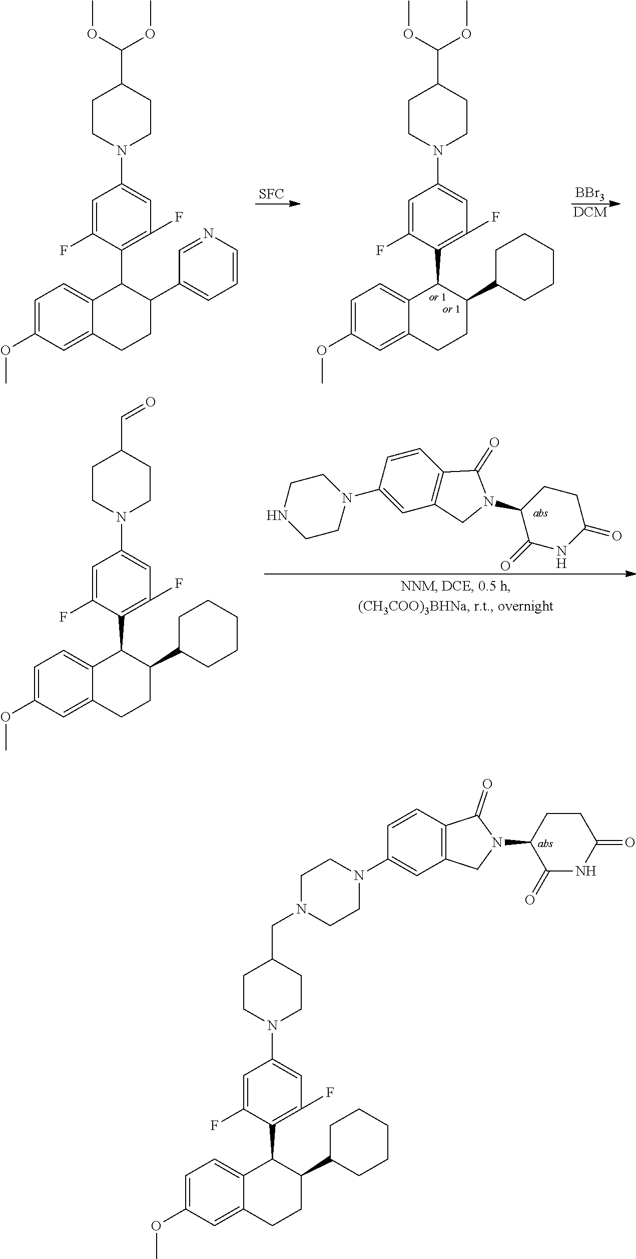

Step 1: The 1-(4-(2-cyclohexyl-6-methoxy-1,2,3,4-tetrahydronaphthalen-1-yl)-3,5-difluorophenyl)-4-(dimethoxymethyl)piperidine (600 mg, 1 eq., 1.17 mmol) was purified by SFC(Column: DAICEL CHIRALCEL OD-H (250 mm*30 mm, 10 um); Mobile phase: 0.1% NH₃H₂O ETOH; from 25% to 25%; Flow rate: 70 mL/min) to give 1-(4-((1S,2R)-2-cyclohexyl-6-methoxy-1,2,3,4-tetrahydronaphthalen-1-yl)-3,5-difluorophenyl)-4-(dimethoxymethyl)piperidine (240 mg, 467 μmol, 40.0%) both as white solid.

LC-MS (ESI⁺) m/z: 514.4 (M+H)⁺.

Step 2: To a solution of 1-(4-((1S,2R)-2-cyclohexyl-6-methoxy-1,2,3,4-tetrahydronaphthalen-1-yl)-3,5-difluorophenyl)-4-(dimethoxymethyl)piperidine (200 mg, 1 eq., 389 μmol) in DCM (10 mL) was added BBr₃ (488 mg, 184 μL, 5 eq., 1.95 mmol). The mixture was stirred at 20° C. for 2 hour and then water (1 mL) in DCM (10 mL) was added dropwise at 20° C., The resulting mixture was stirred at 20° C. for 30 min. The mixture was concentrated and then water (20 mL) was added. The mixture was extracted with EtOAc (10 mL×3), The combined organic layers were washed with brine (20 mL), dried over Na₂SO₄, filtered and concentrated. The mixture was concentrated in vacuo to give crude product. No further purification as it is used for the next step directly. LC-MS (ESI+) m/z: 454.2 (M+H)+.

Step 3: To a solution of 1-(4-((1S,2R)-2-cyclohexyl-6-hydroxy-1,2,3,4-tetrahydronaphthalen-1-yl)-3,5-difluorophenyl)piperidine-4-carbaldehyde (80.0 mg, 1 eq., 176 μmol) and (R)-3-(1-oxo-5-(piperazin-1-yl)isoindolin-2-yl)piperidine-2,6-dione (81.1 mg, 1.4 eq., 247 μmol) in DCE (10 mL) was added dropwise 4-methylmorpholine (17.8 mg, 1 eq., 176 μmol) to pH=8 at 20° C. After addition, the mixture was stirred at this temperature for 30 min, and then sodium triacetoxyhydroborate (93.5 mg, 2.5 eq., 441 μmol) was added dropwise at 20° C. The resulting mixture was stirred at 20° C. for 3 hour. The crude was purified by prep HPLC together (YMC-Triart Prep C18 150*40 mm*7 um, water(FA)-ACN as a mobile phase, from 40% to 70%, Gradient Time (min): 15, Flow Rate (ml/min): 25) to give (S)-3-(5-(4-((1-(4-((1S,2R)-2-cyclohexyl-6-hydroxy-1,2,3,4-tetrahydronaphthalen-1-yl)-3,5-difluorophenyl)piperidin-4-yl)methyl)piperazin-1-yl)-1-oxoisoindolin-2-yl)piperidine-2,6-dione (53.6 mg, 70.0 μmol, 39.7%) as a white solid.

LC-MS (ESI+) m/z: 766.3 (M+H)+.

¹H NMR (400 MHZ, DMSO-d₆) δ: ppm 10.95 (s, 1H), 9.00 (s, 1H), 8.13 (s, 1H), 7.54-7.59 (m, 1H), 7.23-7.39 (m, 1H), 7.02-7.19 (m, 2H), 6.59 (d, J=8.34 Hz, 1H), 6.45 (br d, J=2.03 Hz, 2H), 6.40 (dd, J=8.23, 2.38 Hz, 1H), 5.06 (dd, J=13.23, 5.01 Hz, 1H), 4.30-4.39 (m, 2H), 4.18-4.27 (m, 1H), 3.72 (br d, J=11.56 Hz, 2H), 3.51 (br s, 4H), 2.99-3.30 (m, 4H), 2.82-2.95 (m, 2H), 2.65-2.79 (m, 3H), 2.65-2.65 (m, 1H), 2.59 (br d, J=16.81 Hz, 2H), 2.37 (br dd, J=13.05, 4.47 Hz, 1H), 1.92-1.99 (m, 1H), 1.69-1.89 (m, 6H), 1.66 (br d, J=10.01 Hz, 1 H), 1.55 (br d, J=6.79 Hz, 3H), 1.19-1.26 (m, 2H), 1.05-1.19 (m, 4H), 0.92-1.04 (m, 2H), 0.75-0.89 (m, 1H).

### Example 221. Preparation of (I-118) (S)-3-(5-(4-((1-(4-((1R,2S)-2-cyclohexyl-6-hydroxy-1,2,3,4-tetrahydronaphthalen-1-yl)-3,5-difluorophenyl)piperidin-4-yl)methyl)piperazin-1-yl)-1-oxoisoindolin-2-yl)piperidine-2,6-dione

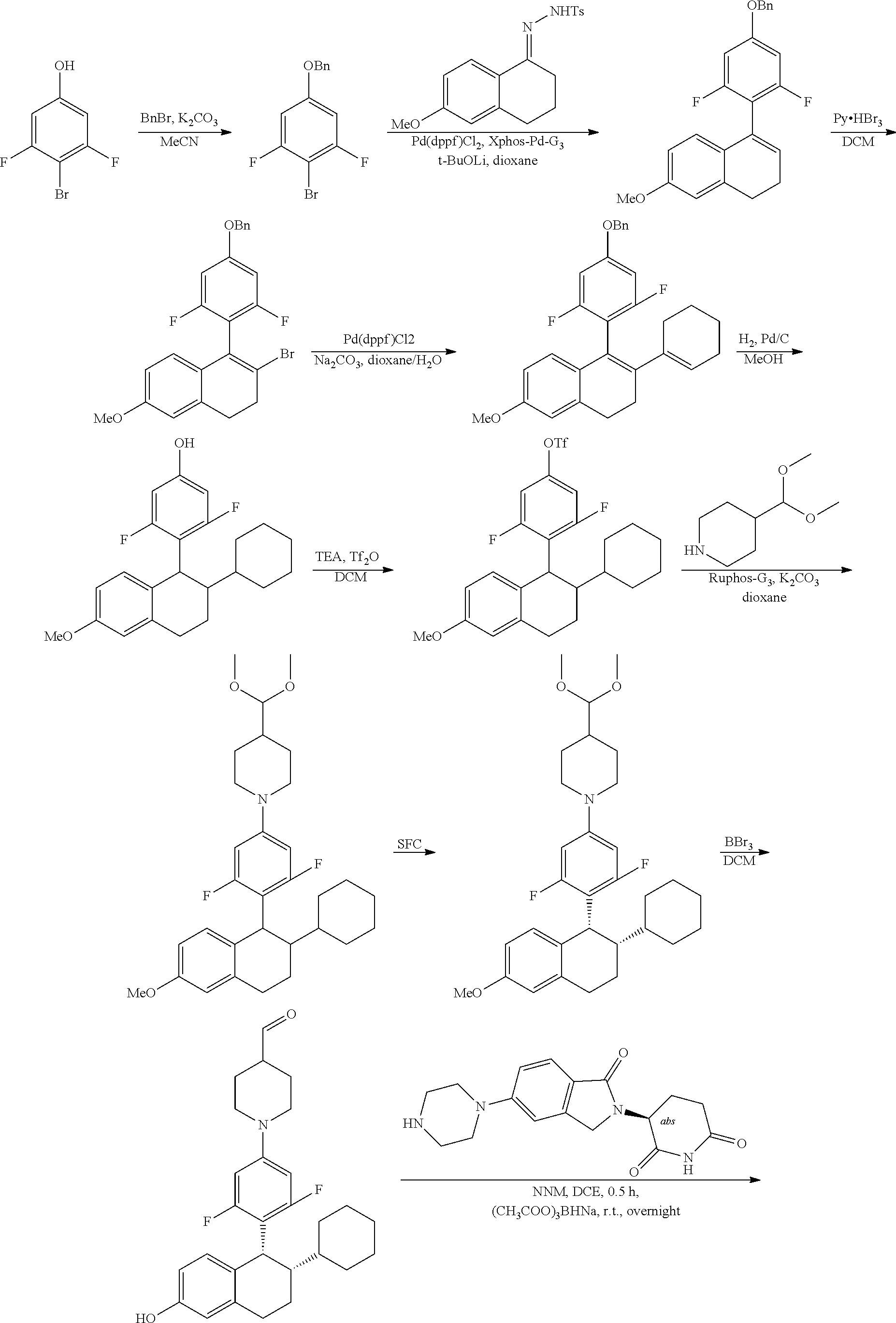

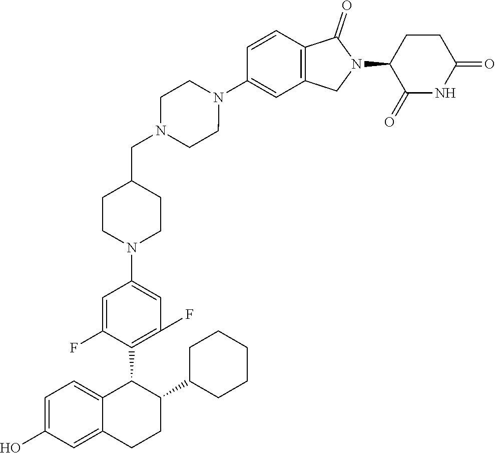 [SMILES: O=C1CC[C@H](N2Cc3cc(N4CCN(CC5CCN(c6cc(F)c([C@H]7c8ccc(O)cc8CC[C@H]7C7CCCCC7)c(F)c6)CC5)CC4)ccc3C2=O)C(=O)N1]

Step 1: To a solution of 4-bromo-3,5-difluorophenol (10 g, 1 eq., 48 mmol) and K₂CO₃ (20 g, 3 eq., 0.14 mol) in MECN (120 mL) was added (bromomethyl)benzene (9.0 g, 1.1 eq., 53 mmol). The mixture was stirred at 70° C. for 3 hour. TLC (PE/EtOAc=10/1, Rf=0.8) showed the reaction was completed. The reaction mixture was quenched by addition EA 60 mL at 20° C., and then diluted with water 30 mL and extracted with EA 150 mL (50 mL*3). The combined organic layers were washed with EA 60 mL (20 mL*3), dried over Na₂SO₄, filtered and concentrated under reduced pressure to give a residue. The crude material was purified on silica gel column chromatography (from PE/EtOAc=1/0 to 1/0, TLC: PE/EtOAc=10/1, Rf=0.8) to yield 5-(benzyloxy)-2-bromo-1,3-difluorobenzene (12 g, 40 mmol, 84%) as a transparent oil.

¹H NMR (400 MHZ, METHANOL-d₄) δ: ppm 7.23-7.53 (m, 5H), 6.70-6.81 (m, 2H), 5.06 (s, 2H).

Step 2: A mixture of 5-(benzyloxy)-2-bromo-1,3-difluorobenzene (5.00 g, 1 eq., 16.7 mmol), (E)-N′-(6-methoxy-3,4-dihydronaphthalen-1 (2H),-ylidene)-4-methylbenzenesulfonohydrazide (6.91 g, 1.2 eq., 20.1 mmol), XPhos Pd G3 (707 mg, 0.05 eq., 836 μmol), PdCl₂(dppf) adduct (683 mg, 0.05 eq., 836 μmol) and Lithium 2-methyl-2-propanolate (3.35 g, 3.89 mL, 2.5 eq., 41.8 mmol) in Dioxane (300 mL) was degassed and purged with N₂ for 3 times, and then the mixture was stirred at 100° C. for 12 hour under N₂ atmosphere. LCMS showed the reaction was completed. The reaction mixture was quenched by addition EA 100 mL at 20° C., and then diluted with water 100 mL and extracted with EA 300 mL (100 mL*3). The combined organic layers were washed with EA 150 mL (50 mL*3), dried over Na2So4, filtered and concentrated under reduced pressure to give a residue. The crude material was purified on silica gel column chromatography (from PE/EtOAc=1/0 to 1/0, TLC: PE/EtOAc=10/1, Rf=0.8) to yield 4-(4-(benzyloxy)-2,6-difluorophenyl)-7-methoxy-1,2-dihydronaphthalene (5.0 g, 13 mmol, 79%) as a yellow solid.

LC-MS (ESI+) m/z: 379.2 (M+H)+

¹H NMR (500 MHz, DMSO-d₆) δ: ppm 7.46-7.50 (m, 2H), 7.40-7.45 (m, 2H), 7.33-7.39 (m, 1H), 6.86-6.93 (m, 2H), 6.82 (d, J=2.59 Hz, 1H), 6.66 (dd, J=8.47, 2.67 Hz, 1H), 6.53 (d, J=8.54 Hz, 1H), 5.94 (t, J=4.58 Hz, 1H), 5.16 (s, 2H), 3.73 (s, 3H), 2.80 (t, J=8.09 Hz, 2H), 2.31-2.41 (m, 2H).

Step 3: To a solution of 4-(4-(benzyloxy)-2,6-difluorophenyl)-7-methoxy-1,2-dihydronaphthalene (3.2 g, 1 eq., 8.5 mmol) in DCM (40 mL) was added Pyridinium bromide perbromide (3.0 g, 1.1 eq., 9.3 mmol). The mixture was stirred at 20° C. for 1.5 hour. LCMS showed the reaction was completed. The reaction mixture was quenched by addition water 30 mL at 20° C., and then diluted with water 30 mL and extracted with DCM 90 mL (30 mL*3). The combined organic layers were washed with DCM 30 mL (10 mL*3), dried over Na₂SO₄, filtered and concentrated under reduced pressure to give a residue. The crude material was purified on silica gel column chromatography (from PE/EtOAc=I/O to 20/1) to yield 4-(4-(benzyloxy)-2,6-difluorophenyl)-3-bromo-7-methoxy-1,2-dihydronaphthalene (2.6 g, 5.8 mmol, 69%) as a yellow solid.

LC-MS (ESI+) m/z: 457.1 (M+H)+.

¹H NMR (400 MHZ, DMSO-d₆) δ: ppm 7.47-7.52 (m, 2H), 7.40-7.46 (m, 2H), 7.35-7.40 (m, 1H), 6.95 (d, J=9.42 Hz, 2H), 6.84 (d, J=2.62 Hz, 1H), 6.67 (dd, J=8.52, 2.68 Hz, 1H), 6.48 (d, J=8.58 Hz, 1H), 5.17 (s, 2H), 3.73 (s, 3H), 2.96-3.02 (m, 2H), 2.88-2.95 (m, 2H).

Step 4: A mixture of 4-(4-(benzyloxy)-2,6-difluorophenyl)-3-bromo-7-methoxy-1,2-dihydronaphthalene (3.6 g, 1 eq., 7.9 mmol), cyclohex-1-en-1-ylboronic acid (1.1 g, 1.1 eq., 8.7 mmol), PdCl₂(dppf) (0.58 g, 0.1 eq., 0.79 mmol) and sodium carbonate (2.5 g, 3 eq., 24 mmol) in Dioxane (20 mL) and water (4 mL) was degassed and purged with N₂ for 3 times, and then the mixture was stirred at 80° C. for 8 hour under N₂ atmosphere. LCMS showed the reaction was completed. The reaction mixture was quenched by addition EA 30 mL at 20° C., and then diluted with water 30 mL and extracted with EA 90 mL (30 mL*3). The combined organic layers were washed with EA 30 mL (10 mL*3), dried over Na₂So4, filtered and concentrated under reduced pressure to give a residue. The crude material was purified on silica gel column chromatography (from PE/EtOAc=I/O to I/O) to yield 4-(4-(benzyloxy)-2,6-difluorophenyl)-3-(cyclohex-1-en-1-yl)-7-methoxy-1,2-dihydronaphthalene (3.2 g, 7.0 mmol, 89%) as a yellow solid.

LC-MS (ESI+) m/z: 459.2 (M+H)+

¹H NMR (400 MHZ, DMSO-d₆) δ: ppm 7.44-7.50 (m, 2H), 7.41 (t, J=7.21 Hz, 2H), 7.32-7.38 (m, 1H), 6.75-6.88 (m, 3H), 6.63 (dd, J=8.46, 2.62 Hz, 1H), 6.45 (d, J=8.46 Hz, 1H), 5.24 (brs, 1H), 5.11-5.12 (m, 1H), 5.15 (s, 1H), 3.71 (s, 3H), 2.79 (t, J=7.93 Hz, 2H), 2.39 (t, J=7.93 Hz, 2H), 1.91 (br s, 2H), 1.76 (br d, J=3.58 Hz, 2H), 1.44-1.50 (m, 2H), 1.20-1.29 (m, 2H).

Step 5: To a solution of 4-(4-(benzyloxy)-2,6-difluorophenyl)-3-(cyclohex-1-en-1-yl)-7-methoxy-1,2-dihydronaphthalene (4.0 g, 1 eq., 8.7 mmol) in MeOH (30 mL) was added Pd/C (10%, 2 g) under N₂ atmosphere. The suspension was degassed and purged with H2 for 3 times. The mixture was stirred under H₂ (50 Psi) at 50° C. for 24 hour. LCMS showed the reaction was completed. After filtration, the filtrate was concentrated give crude product. The crude was purified by prep.HPLC together (Boston Uni C18 40*150*5 um, water(FA)-ACN as a mobile phase, from 70% to 100%, Gradient Time (min): 10, Flow Rate (ml/min): 60) to give 4-(2-cyclohexyl-6-methoxy-1,2,3,4-tetrahydronaphthalen-1-yl)-3,5-difluorophenol (750 mg, 2.01 mmol, 23%) as a white solid.

LC-MS (ESI+) m/z: 373.3 (M+H)+

¹H NMR (400 MHZ, DMSO-d₆) δ: ppm 9.96-10.39 (m, 1H), 6.29-6.74 (m, 5H), 4.09-4.45 (m, 1H), 2.68-2.95 (m, 2H), 1.47-1.92 (m, 7H), 0.76-1.23 (m, 7H).

Step 6: To a solution of 4-(2-cyclohexyl-6-methoxy-1,2,3,4-tetrahydronaphthalen-1-yl)-3,5-difluorophenol (720 mg, 1 eq., 1.93 mmol) and TEA (587 mg, 808 μL, 3 eq., 5.80 mmol) in DCM (10 mL) was added trifluoromethanesulfonic anhydride (818 mg, 1.5 eq., 2.90 mmol). The mixture was stirred at 0° C. for 2 hour. LCMS showed the reaction was completed. The reaction mixture was quenched by addition water 20 mL at 20° C., and then diluted with water 10 mL and extracted with DCM 30 mL (10 mL*3). The combined organic layers were washed with DCM 15 mL (5 mL*3), dried over Na₂SO₄, filtered and concentrated under reduced pressure to give a residue. The crude material was purified on silica gel column chromatography (from PE/EtOAc=I/O to 40/1) to yield 4-(2-cyclohexyl-6-methoxy-1,2,3,4-tetrahydronaphthalen-1-yl)-3,5-difluorophenyl trifluoromethanesulfonate (460 mg, 912 μmol, 47.2%) as a colourless oil.

LC-MS (ESI+) m/z: 505.2 (M+H)+

¹H NMR (400 MHZ, DMSO-d₆) δ: ppm 7.08-7.90 (m, 2H), 6.49-6.80 (m, 3H), 4.56 (d, J=5.84 Hz, 1H), 3.66-3.72 (m, 3H), 2.75-2.98 (m, 2H), 1.48-1.97 (m, 7H), 0.81-1.14 (m, 7H), −2.64-−2.62 (m, 1H).

Step 7: A mixture of 4-(2-cyclohexyl-6-methoxy-1,2,3,4-tetrahydronaphthalen-1-yl)-3,5-difluorophenyl trifluoromethanesulfonate (460 mg, 1 eq., 912 μmol), 4-(dimethoxymethyl)piperidine (218 mg, 1.5 eq., 1.37 mmol), potassium carbonate (378 mg, 3 eq., 2.74 mmol) and (2-Dicyclohexylphosphino-2′,6′-diisopropoxy-1,1′-biphenyl) [2-(2′-amino-1,1′-biphenyl)]palladium(II)methanesulfonate (153 mg, 0.2 eq., 182 μmol) in was degassed and purged with N₂ for 3 times, and then the mixture was stirred at 100° C. for 12 hour under N₂ atmosphere. LCMS showed the reaction was completed. The reaction mixture was quenched by addition EA 10 Ml at 20° C., and then diluted with water 30 Ml and extracted with EA 30 Ml (10 Ml*3). The combined organic layers were washed with EA 30 Ml (10 Ml*3), dried over Na₂So4, filtered and concentrated under reduced pressure to give a residue. The crude material was purified on silica gel column chromatography (from pure PE to PE/EA=9/1) to as give 1-(4-(2-cyclohexyl-6-methoxy-1,2,3,4-tetrahydronaphthalen-1-yl)-3,5-difluorophenyl)-4-(dimethoxymethyl)piperidine (400 mg, 779 μmol, 85.4%) a yellow oil.

LC-MS (ESI+) m/z: 514.4 (M+H)+

¹H NMR (400 MHz, DMSO-d₆) δ: ppm 7.08-7.90 (m, 2H), 6.49-6.80 (m, 3H), 4.56 (d, J=5.84 Hz, 1H), 3.66-3.72 (m, 3H), 2.75-2.98 (m, 2H), 1.48-1.97 (m, 7H), 0.81-1.14 (m, 7H), −2.64-−2.62 (m, 1H).

Step 8: The 1-(4-(2-cyclohexyl-6-methoxy-1,2,3,4-tetrahydronaphthalen-1-yl)-3,5-difluorophenyl)-4-(dimethoxymethyl)piperidine (600 mg, 1 eq., 1.17 mmol) was purified by SFC(Column: DAICEL CHIRALCEL OD-H (250 mm*30 mm, 10 um); Mobile phase: 0.1% NH₃H₂O ETOH; from 25% to 25%; Flow rate: 70 mL/min) to give 1-(4-((1R,2S)-2-cyclohexyl-6-methoxy-1,2,3,4-tetrahydronaphthalen-1-yl)-3,5-difluorophenyl)-4-(dimethoxymethyl)piperidine (230 mg, 448 μmol, 38.3%) as white solid.

LC-MS (ESI+) m/z: 514.4 (M+H)+.

Step 9: To a solution of 1-(4-((1R,2S)-2-cyclohexyl-6-methoxy-1,2,3,4-tetrahydronaphthalen-1-yl)-3,5-difluorophenyl)-4-(dimethoxymethyl)piperidine (200 mg, 1 eq., 389 μmol) in DCM (10 mL) was added BBr₃ (488 mg, 184 μL, 5 eq., 1.95 mmol). The mixture was stirred at 20° C. for 2 hour and then water (2 mL) in DCM (10 mL) was added dropwise at 20° C., The resulting mixture was stirred at 20° C. for 30 min. LCMS showed the reaction was completed. The mixture was concentrated and then water (20 mL) was added. The mixture was extracted with EtOAc (20 mL×3). The combined organic layers were washed with brine (20 mL), dried over Na₂SO₄, filtered and concentrated. The mixture was concentrated in vacuo to give crude product. No further purification as it is used for the next step directly.

LC-MS (ESI+) m/z: 454.2 (M+H)+.

Step 10: To a solution of 1-(4-((1R,2S)-2-cyclohexyl-6-hydroxy-1,2,3,4-tetrahydronaphthalen-1-yl)-3,5-difluorophenyl)piperidine-4-carbaldehyde (80 mg, 1 eq., 0.18 mmol) and (R)-3-(1-oxo-5-(piperazin-1-yl)isoindolin-2-yl)piperidine-2,6-dione (81 mg, 1.4 eq., 0.25 mmol) in DCE (10 mL) was added dropwise 4-methylmorpholine (18 mg, 1 eq., 0.18 mmol) to pH=8 at 20° C. After addition, the mixture was stirred at this temperature for 0.5 hour, and then sodium triacetoxyhydroborate (93 mg, 2.5 eq., 0.44 mmol) was added dropwise at 20° C. The resulting mixture was stirred at 20° C. for 3 hour. LCMS showed the reaction was completed. The crude was purified by prep. HPLC together (YMC-Triart Prep C18 150*40 mm*7 um, water(FA)-ACN as a mobile phase, from 40% to 70%, Gradient Time (min): 15, Flow Rate (ml/min): 25) to give (S)-3-(5-(4-((1-(4-((1R,2S)-2-cyclohexyl-6-hydroxy-1,2,3,4-tetrahydronaphthalen-1-yl)-3,5-difluorophenyl)piperidin-4-yl)methyl)piperazin-1-yl)-1-oxoisoindolin-2-yl)piperidine-2,6-dione (50.8 mg, 66.3 μmol, 38%) as a white solid.

LC-MS (ESI+) m/z: 766.3 (M+H)+

¹H NMR (400 MHZ, METHANOL-d₄) δ: ppm 8.11-8.16 (m, 1H), 7.65-7.72 (m, 1H), 7.36-7.46 (m, 1H), 7.13-7.19 (m, 2H), 6.64 (d, J=8.34 Hz, 1H), 6.50 (d, J=2.50 Hz, 1H), 6.41-6.45 (m, 2H), 5.11 (dd, J=13.35, 5.13 Hz, 1H), 4.37-4.48 (m, 3H), 3.74 (br d, J=12.52 Hz, 2H), 3.61 (br s, 4H), 3.32-3.44 (m, 4H), 2.98-3.07 (m, 2H), 2.84-2.96 (m, 2H), 2.73-2.84 (m, 4H), 2.46 (qd, J=13.13, 4.59 Hz, 1H), 2.12-2.20 (m, 1H), 2.01-2.09 (m, 1H), 1.88 (br d, J=10.37 Hz, 3H), 1.75-1.84 (m, 2H), 1.71 (br d, J=9.30 Hz, 2H), 1.60 (br d, J=10.01 Hz, 2H), 1.36-1.46 (m, 2H), 1.19-1.32 (m, 3H), 1.00-1.16 (m, 3H), 0.82-0.95 (m, 1H).

### Example 222. Preparation of (I-108) (R)-3-(5-(4-((1-(4-((1S,2R)-6-hydroxy-2-(pyridin-3-yl)-1,2,3,4-tetrahydronaphthalen-1-yl)phenyl)piperidin-4-yl)methyl)piperazin-1-yl)-1-oxoisoindolin-2-yl)piperidine-2,6-dione

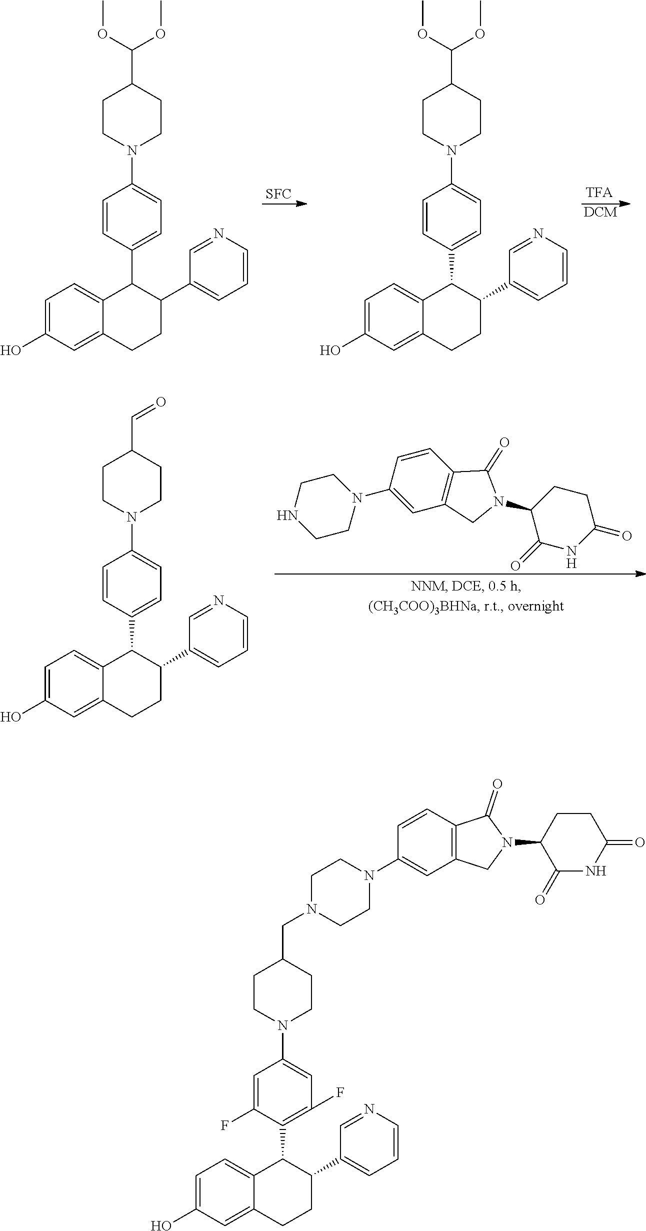

Step 1: The starting material was purified by SFC(Column: DAICEL CHIRALPAK AS (250 mm*30 mm, 10 um)); Mobile phase: 0.1% NH₃H₂O ETOH; from 30% to 30%; Flow rate: 90 mL/min) to give (5S,6R)-5-(4-(4-(dimethoxymethyl)piperidin-1-yl)phenyl)-6-(pyridin-3-yl)-5,6,7,8-tetrahydronaphthalen-2-ol (76 mg, 0.17 mmol, 36%) as white solid.

LC-MS (ESI⁺) m/z: 459.3 (M+H)⁺.

Step 2: To a solution of (5S,6R)-5-(4-(4-(dimethoxymethyl)piperidin-1-yl)phenyl)-6-(pyridin-3-yl)-5,6,7,8-tetrahydronaphthalen-2-ol (60 mg, 1 Eq., 0.13 mmol) in DCM (3 mL) was added TFA (45 mg, 30 μL, 3 Eq., 0.39 mmol). The mixture was stirred at 25° C. for 4 hour. LCMS showed the reaction was completed. The mixture was concentrated in vacuo to give crude product. No further purification as it is used for the next step directly.

LC-MS (ESI+) m/z: 413.2 (M+H)+.

Step 3: To a solution of 1-(4-((1S,2R)-6-hydroxy-2-(pyridin-3-yl)-1,2,3,4-tetrahydronaphthalen-1-yl)phenyl)piperidine-4-carbaldehyde (30 mg, 1 Eq., 73 μmol) and (R)-3-(1-oxo-5-(piperazin-1-yl)isoindolin-2-yl)piperidine-2,6-dione (36 mg, 1.5 Eq., 0.11 mmol) in DCE (3 mL) was added dropwise 4-methylmorpholine (7.4 mg, 1 Eq., 73 μmol) to pH=8 at 20° C. After addition, the mixture was stirred at this temperature for 0.5 hour, and then sodium triacetoxyhydroborate (31 mg, 2 Eq., 0.15 mmol) was added dropwise at 20° C. The resulting mixture was stirred at 20° C. for 3 hour. LCMS showed the reaction was completed. The crude was purified by prep. HPLC together (Welch Xtimate C18 150*25 mm*5 um, water (NH₃H₂O+NH₄HCO₃)-ACN as a mobile phase, from 35% to 65%, Gradient Time (min): 11, Flow Rate (ml/min): 25) to give (R)-3-(5-(4-((1-(4-((1S,2R)-6-hydroxy-2-(pyridin-3-yl)-1,2,3,4-tetrahydronaphthalen-1-yl)phenyl)piperidin-4-yl)methyl)piperazin-1-yl)-1-oxoisoindolin-2-yl)piperidine-2,6-dione (27.4 mg, 37.8 μmol, 52%) as a white solid.

LC-MS (ESI+) m/z: 725.3 (M+H)+

¹H NMR (500 MHZ, METHANOL-d₄) δ: ppm 11.80-11.84 (m, 1H), 8.30 (br s, 1H), 8.01 (br s, 1H), 7.65 (d, J=8.54 Hz, 1H), 7.22 (br s, 2H), 6.94-7.17 (m, 2H), 6.65-6.74 (m, 4H), 6.55 (dd, J=8.39, 2.44 Hz, 1H), 6.38 (d, J=8.54 Hz, 2H), 5.11 (dd, J=13.28, 5.19 Hz, 1H), 4.58 (s, 1H), 4.28-4.54 (m, 2H), 4.21 (d, J=5.19 Hz, 1H), 3.52-3.59 (m, 2H), 3.35-3.45 (m, 5H), 3.00-3.11 (m, 2H), 2.84-2.96 (m, 1H), 2.74-2.84 (m, 1H), 2.52-2.72 (m, 6H), 2.47 (qd, J=13.25, 4.81 Hz, 1H), 2.31 (br d, J=7.02 Hz, 2H), 2.12-2.20 (m, 1H), 1.88 (br d, J=12.51 Hz, 2H), 1.66-1.82 (m, 2H), 1.11-1.49 (m, 3H). Stereochemistry was arbitrarily assigned

### Example 223. Preparation of (I-133) (S)-3-(5-(4-((1-(4-((1R,2S)-6-hydroxy-2-(pyridine-4-yl)-1,2,3,4-tetrahydronaphthalen-1-yl)phenyl)piperidin-4-yl)methyl)piperazin-1-yl)-1-oxoisoindolin-2-yl)piperidine-2,6-dione

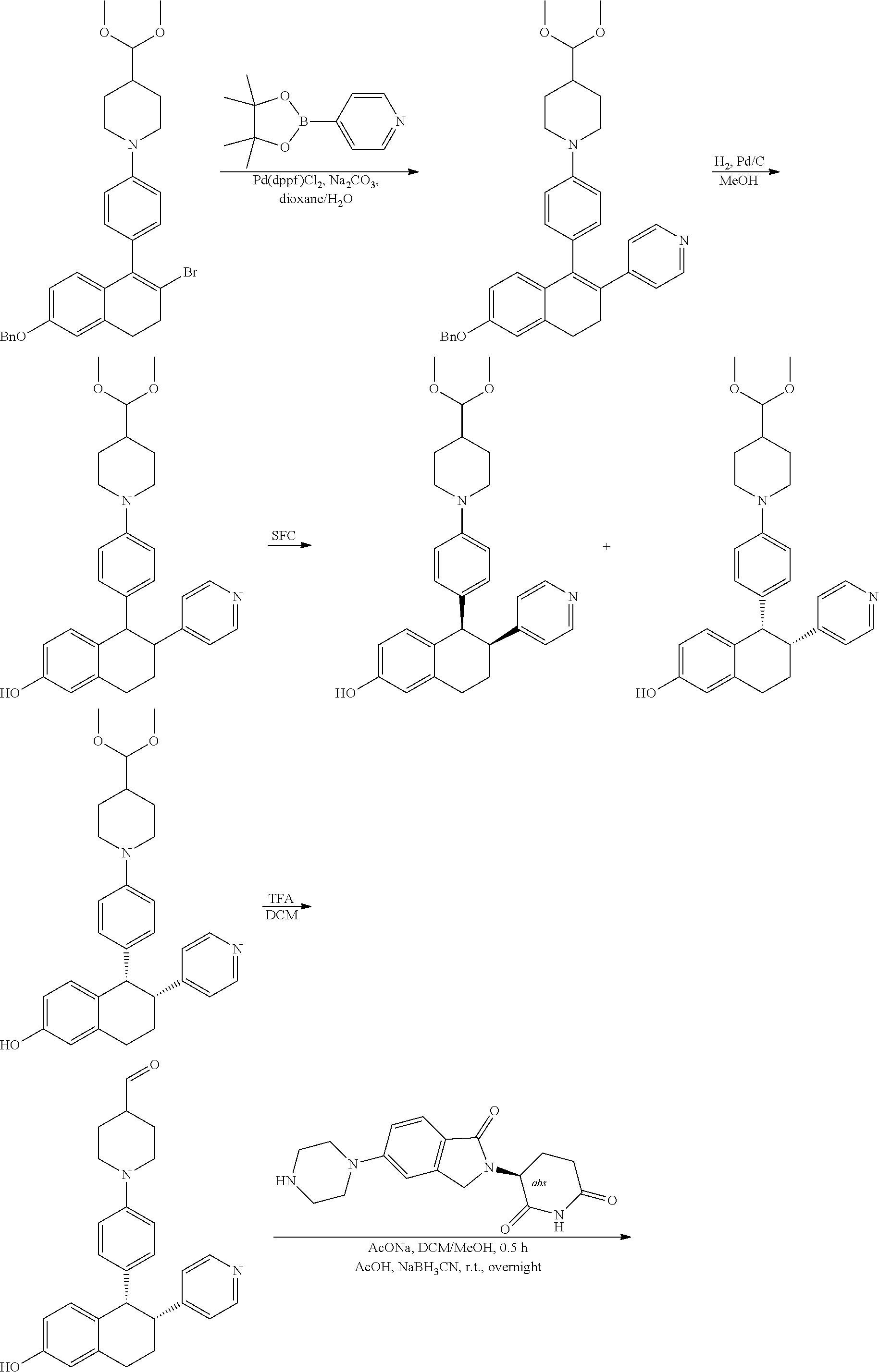

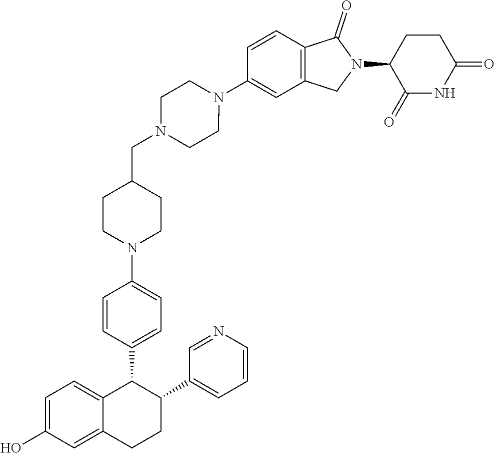 [SMILES: O=C1CC[C@H](N2Cc3cc(N4CCN(CC5CCN(c6ccc([C@H]7c8ccc(O)cc8CC[C@H]7c7cccnc7)cc6)CC5)CC4)ccc3C2=O)C(=O)N1]

Step 1: To a mixture of 1-(4-(6-(benzyloxy)-2-bromo-3,4-dihydronaphthalen-1-yl)phenyl)-4-(dimethoxymethyl)piperidine (3.00 g, 1 Eq, 5.47 mmol), 4-(4,4,5,5-tetramethyl-1,3,2-dioxaborolan-2-yl)pyridine (1.12 g, 1 Eq, 5.47 mmol), PdCl₂(dppf) (800 mg, 0.2 Eq, 1.09 mmol) and K₂CO₃ (2.27 g, 3 Eq, 16.4 mmol) in Dioxane (100 mL) and H₂O (2 mL) was added reagent in one portion at 25° C. under N₂. The mixture was suction air for 3 times under N₂, then heated to 85° C. and stirred for 8 hours. TLC showed the reaction was completed. The mixture was cooled to 20° C., solid was filtered and concentrated in reduced pressure at 25° C. The residue was poured into ice-water (w/w=1/1) (200 mL) and stirred for 5 min. The aqueous phase was extracted with ethyl acetate (100 mL*2). The combined organic phase was washed with brine (50 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=5/1, N/1) to afford 5-(6-(benzyloxy)-2-(1392yridine-4-yl)-3,4-dihydronaphthalen-1-yl)-2-(4-(dimethoxymethyl)piperidin-1-yl)benzene-1-ylium (2.54 g, 4.6 mmol, 84%, 99% Purity) as yellow solid.

Step 2: To a mixture of 4-(6-(benzyloxy)-1-(4-(4-(dimethoxymethyl)piperidin-1-yl)phenyl)-3,4-dihydronaphthalen-2-yl)pyridine (2.25 g, 1 Eq, 4.12 mmol) and Pd—C (438 mg, 1 Eq, 4.12 mmol) in Ethyl acetate (100 mL) was added reagent in one portion at 25° C. under H₂. The mixture was stirred at 25° C. for 5 min, then heated to 80° C. and stirred for 24 hours at 50 psi. TLC showed the reaction was completed. The mixture was cooled to 20° C., add 200 ml solution (DCM and MeOH=1:1) to the mixture and filer solid and concentrated in reduced pressure at 30° C. to afford 5-(4-(4-(dimethoxymethyl)piperidin-1-yl)phenyl)-6-(pyridin-4-yl)-5,6,7,8-tetrahydronaphthalen-2-ol (1.68 g, 3.88 mmol, 94.26%) as yellow solid.

Step 3: To a mixture of 5-(4-(4-(dimethoxymethyl)piperidin-1-yl)phenyl)-6-(pyridin-4-yl)-5,6,7,8-tetrahydronaphthalen-2-ol (1.00 g, 1 Eq, 2.18 mmol) in 5-(4-(4-(dimethoxymethyl)piperidin-1-yl)phenyl)-6-(pyridin-4-yl)-5,6,7,8-tetrahydronaphthalen-2-ol (1.00 g, 1 Eq, 2.18 mmol). SFC to give (5R,6S)-5-(4-(4-(dimethoxymethyl)piperidin-1-yl)phenyl)-6-(pyridin-4-yl)-5,6,7,8-tetrahydronaphthalen-2-ol (350 mg, 763 μmol, 70.0%) and as yellow solid.

Step 4: To a mixture To a mixture of To a mixture of (5S,6R)-5-(4-(4-(dimethoxymethyl)piperidin-1-yl)phenyl)-6-(1392etrahyd-4-yl)-5,6,7,8-tetrahydronaphthalen-2-ol (100 mg, 1 Eq, 218 μmol) and TFA (24.9 mg, 16.8 uL, 1 Eq, 218 μmol) in DCM (2 mL) was added reagent in one portion. The mixture was stirred at 25° C. for 1 hour. LCMS showed the reaction was completed. The mixture was cooled to 25° C. and concentrated in reduced to afford 1-(4-((1S, 2R)-6-hydroxy-2-(pyridin-4-yl)-1,2,3,4-tetrahydronaphthalen-1-yl)phenyl)piperidine-4-carbaldehyde (104 mg, 153 μmol, 70.0%, 60.543% Purity) as yellow solid. Directly to next step.

Step 5: To a mixture of 1-(4-((1R,2S)-6-hydroxy-2-(1393yridine-4-yl)-1,2,3,4-tetrahydronaphthalen-1-yl)phenyl)piperidine-4-carbaldehyde (100 mg, 1 Eq, 242 μmol), (S)-3-(1-oxo-5-(piperazin-1-yl)isoindolin-2-yl)piperidine-2,6-dione (159 mg, 2 Eq, 485 μmol) in DCM (15 mL) was added reagent in one portion at 25° C. The mixture was stirred at 25° C. for 5 min, then add 4-methylmorfolin (49.0 mg, 53 μL, 2 Eq, 485 μmol) and Sodium triacetoxyborohydride (154 mg, 108 μL, 3 Eq, 727 μmol) to adjust PH to about 6 and stirred for 3 hours. TLC showed the reaction was completed. The mixture was reduced pressure at 25° C. The residue was further purification by pre-HPLC to afford (S)-3-(5-(4-((1-(4-((1R,2S)-6-hydroxy-2-(1393yridine-4-yl)-1,2,3,4-tetrahydronaphthalen-1-yl)phenyl)piperidin-4-yl)methyl)piperazin-1-yl)-1-oxoisoindolin-2-yl)piperidine-2,6-dione (10.4 mg, 14.34 μmol, 5.92%, 100% Purity) as white solid. The white solid was re-crystallized from water, dried by lyophilization to give a white solid.

### Example 224. Preparation of (I-60) (3S)-3-[5-[4-[[1-[4-[(1S,2R)-6-hydroxy-2-tetralin-6-yl-tetralin-1-yl]phenyl]-4-piperidyl]methyl]piperazin-1-yl]-1-oxo-isoindolin-2-yl]piperidine-2,6-dione

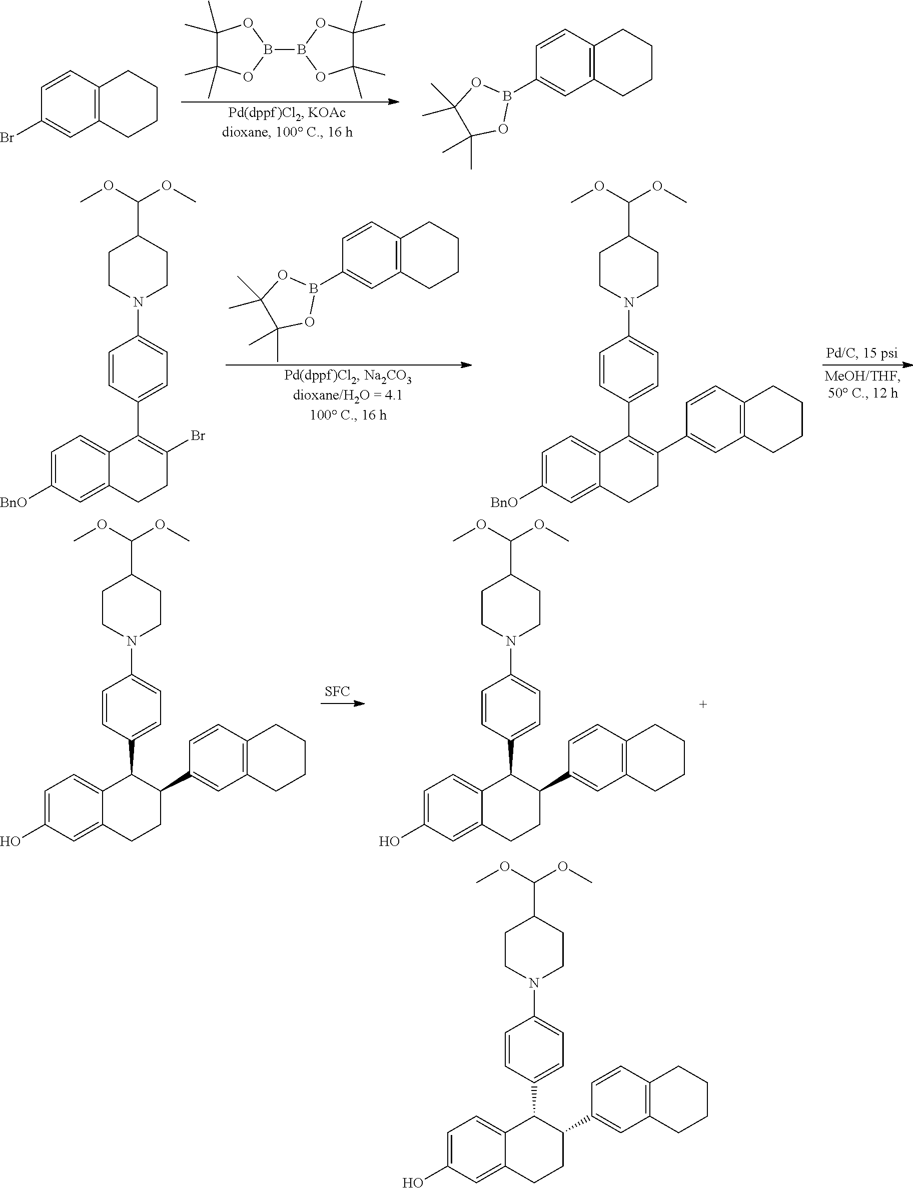

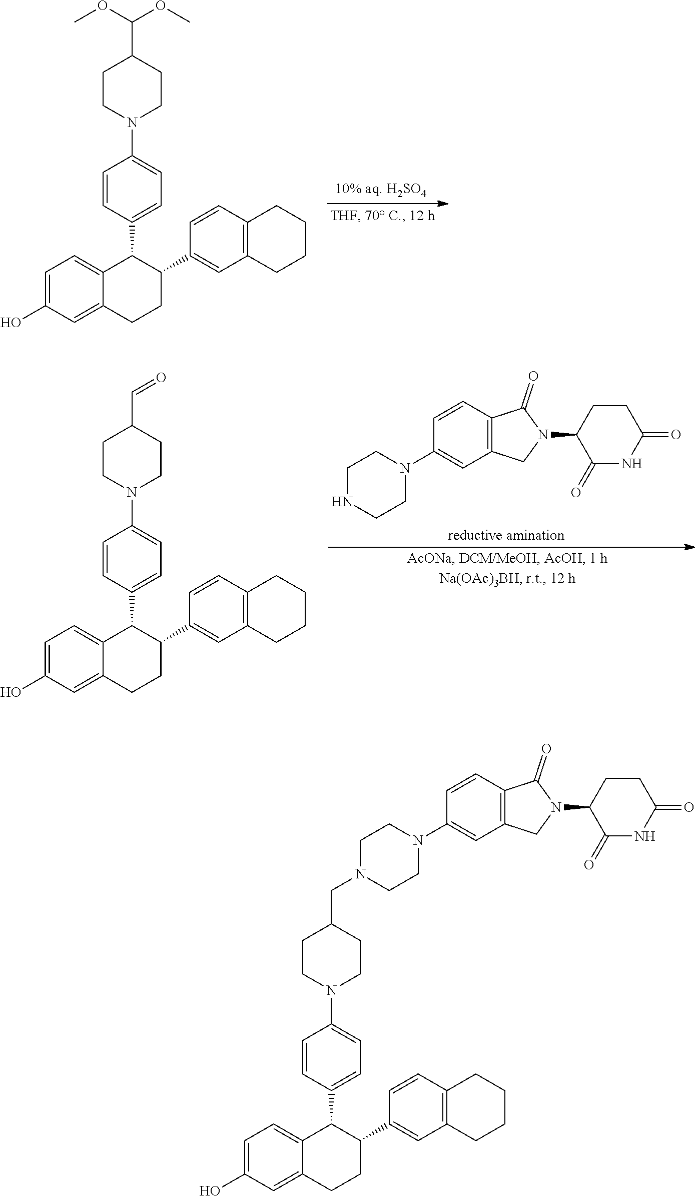 [SMILES: COC(OC)C1CCN(c2ccc([C@H]3c4ccc(O)cc4CC[C@H]3c3ccc4c(c3)CCCC4)cc2)CC1 ; O=CC1CCN(c2ccc([C@H]3c4ccc(O)cc4CC[C@H]3c3ccc4c(c3)CCCC4)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc8c(c7)CCCC8)cc6)CC5)CC4)ccc3C2=O)C(=O)N1]

Step 1: A mixture of 6-bromotetralin (500 mg, 2.37 mmol, 1 eq), 4,4,5,5-tetramethyl-2-(4,4,5,5-tetramethyl-1,3,2-dioxaborolan-2-yl)-1,3,2-dioxaborolane (902.20 mg, 3.55 mmol, 1.5 eq), potassium acetate (464.91 mg, 4.74 mmol, 2 eq), cyclopentyl(diphenyl)phosphane;dichloromethane;dichloropalladium;iron (193.43 mg, 236.86 μmol, 0.1 eq) in dioxane (15 mL) was degassed and purged with N₂ for 3 times, and then the mixture was stirred at 80° C. for 16 hr under N₂ atmosphere. TLC (Petroleum ether/Ethyl acetate=5/1, R_{f}=0.4) showed new spot was formed. The reaction mixture was quenched by addition H₂O 50 mL, and then diluted with 50 mL H₂O and extracted with Ethyl acetate (50 mL*2). The combined organic layer was dried over Na₂SO₄, filtered and concentrated give the crude product, The residue was purified by column chromatography (SiO₂, Petroleum ether/Ethyl acetate=0% to 20%), Compound 4,4,5,5-tetramethyl-2-tetralin-6-yl-1,3,2-dioxaborolane (350 mg, 1.36 mmol, 57.24% yield) was obtained as a white oil.

LC-MS (ESI⁺) m/z: 259.1 (M+H)⁺.

Step 2: A mixture of 1-[4-(6-benzyloxy-2-bromo-3,4-dihydronaphthalen-1-yl)phenyl]-4-(dimethoxymethyl)piperidine (600 mg, 1.09 mmol, 1 eq), 4,4,5,5-tetramethyl-2-tetralin-6-yl-1,3,2-dioxaborolane (282.40 mg, 1.09 mmol, 1 eq), cyclopentyl(diphenyl)phosphane;dichloromethane;dichloropalladium;iron (89.33 mg, 109.39 μmol, 0.1 eq), Na₂CO₃ (347.82 mg, 3.28 mmol, 3 eq) in dioxane (10 mL) and H₂O (2.5 mL) was degassed and purged with N₂ for 3 times, and then the mixture was stirred at 100° C. for 12 hr under N₂ atmosphere. LCMS showed 81% of desired compound was detected. The reaction mixture was quenched by addition 50 mL H2O, and then diluted with 50 mL H₂O and extracted with Ethyl acetate (50 mL*2. The combined organic layer was dried over Na₂SO₄, filtered and concentrated give the crude product, The residue was purified by column chromatography (SiO₂, Petroleum ether/Ethyl acetate=0% to 20%), Compound 1-[4-(6-benzyloxy-2-tetralin-6-yl-3,4-dihydronaphthalen-1-yl)phenyl]-4-(dimethoxymethyl)piperidine (620 mg, 1.03 mmol, 94.50% yield) was obtained as a yellow oil.

LC-MS (ESI⁺) m/z: 600.2 (M+H)⁺.

Step 3: A mixture of 1-[4-[4-(dimethoxymethyl)-1-piperidyl]phenyl]-2-tetralin-6-yl-tetralin-6-ol (527.15 mg, 1.03 mmol, 1 eq), Pd/C (500 mg, 4.71 mmol, 10% purity), in MeOH (5 mL) and THF (5 mL) was degassed and purged with H₂ for 3 times, and then the mixture was stirred at 50° C. for 12 hr under H₂ atmosphere. LCMS showed 84.236% of desired compound was detected. The reaction mixture was filtered and concentrated to dryness in vacuo to give a white solid. The white solid was subjected by SFC: Column: DAICEL CHIRALCEL OD (250 mm*30 mm, 10 um) Mobile phase: [0.1% NH₃H₂O ETOH]; B %: 35%-35%, min Column Temp: 38° C.; Nozzle Pressure: 100 Bar; Nozzle Temp: 60° C.; Evaporator Temp: 20° C.; Trimmer Temp: 25° C.; Wavelength: 220 nm. The aqueous phase was lyophilized to dryness to give Compound (1R,2S)-1-[4-[4-(dimethoxymethyl)-1-piperidyl]phenyl]-2-tetralin-6-yl-tetralin-6-ol (202 mg, 394.77 μmol, 38.32% yield) was obtained as a yellow solid. (1S,2R)-1-(4-(4-(dimethoxymethyl)piperidin-1-yl)phenyl)-1,2,3,4,5′,6′,7′,8′-octahydro-[2,2′-binaphthalen]-6-ol (160 mg, 312.69 μmol, 30.35% yield) was obtained as a yellow solid.

LC-MS (ESI⁺) m/z: 512.2 (M+H)⁺.

Step 4: To a solution of (1S,2R)-1-[4-[4-(dimethoxymethyl)-1-piperidyl]phenyl]-2-tetralin-6-yl-tetralin-6-ol (160 mg, 312.69 μmol, 1 eq) was added H₂SO₄ (10 mL) and THF (10 mL). The mixture was stirred at 70° C. for 12 hr. LCMS showed 92% of desired compound was detected. The reaction mixture was quenched by addition NaHCO₃30 mL at 25° C., and then diluted with H₂O 20 mL and extracted with Ethyl acetate (50 mL*3). The combined organic layers were washed with brine (50 mL), dried over Na₂SO₄, filtered and concentrated under reduced pressure to give the compound as a yellow oil. The crude product 1-[4-[(1S,2R)-6-hydroxy-2-tetralin-6-yl-tetralin-1-yl]phenyl]piperidine-4-carbaldehyde (130 mg, 279.19 μmol, 89.29% yield) was used into the next step without further purification. LC-MS (ESI⁺) m/z: 484.1 (M+H2O)⁺.

Step 5: A mixture of 1-[4-[(1S,2R)-6-hydroxy-2-tetralin-6-yl-tetralin-1-yl]phenyl]piperidine-4-carbaldehyde (130.00 mg, 279.19 μmol, 1 eq), HOAc (69.64 mg, 1.16 mmol, 66.33 uL, 3 eq), NaOAc (231.27 mg, 837.58 μmol, 3 eq) and (3S)-3-(1-oxo-5-piperazin-1-yl-isoindolin-2-yl)piperidine-2,6-dione (139.76 mg, 279.19 μmol, 1 eq, p-TSA salt) in DCM (4 mL) and MeOH (4 mL) was stirred at 20° C. for 1 h, sodium acetate (68.71 mg, 837.58 μmol, 3 eq), Na(OAc)₃BH (118.35 mg, 558.39 μmol, 2 eq) was added to the reaction and then the mixture was stirred at r.t. for 12 hr. The reaction mixture was quenched by addition 50 mL H2O, and then diluted with 50 mL H₂O and extracted with Ethyl acetate 50 mL (50 mL*2). The organic layers were dried with anhydrous Na₂SO₄, filtered, and concentrated to give the crude product. The residue was purified by prep-HPLC (column: Welch Xtimate C18 150*30 mm*5 um; mobile phase: [water(FA)-ACN]; B %: 32%-62%, 7 min). Compound (3S)-3-[5-[4-[[1-[4-[(1S,2R)-6-hydroxy-2-tetralin-6-yl-tetralin-1-yl]phenyl]-4-piperidyl]methyl]piperazin-1-yl]-1-oxo-isoindolin-2-yl]piperidine-2,6-dione (34.4 mg, 44.22 μmol, 15.84% yield) was obtained as a yellow solid.

LC-MS (ESI⁺) m/z: 778.2 (M+H)⁺

HPLC: 99.203% purity at 220 nm

SFC: Rt: 3.280 min; Area: 85.353%; method:
IC_MeOH_DEA_MeCN_50_1ML_15 MlN_10 CM.lcm

¹H NMR (400 MHZ, DMSO-d₆)₂=10.98-10.93 (m, 1H), 9.35-8.92 (m, 1H), 8.20-8.14 (m, 0.590H), 7.55-7.49 (m, 1H), 7.09-7.03 (m, 2H), 6.84-6.77 (m, 1H), 6.67-6.62 (m, 1H), 6.61-6.54 (m, 3H), 6.53-6.46 (m, 2H), 6.42-6.35 (m, 1H), 6.27-6.21 (m, 2H), 5.09-5.01 (m, 1H), 4.36-4.29 (m, 1H), 4.25-4.17 (m, 1H), 4.06 (br d, J=4.5 Hz, 1H), 3.59-3.49 (m, 4H), 3.46-3.35 (m, 6H), 3.15 (br dd, J=2.5, 12.8 Hz, 2H), 2.97-2.83 (m, 3H), 2.69-2.55 (m, 5H), 2.45-2.33 (m, 2H), 2.23-2.16 (m, 2H), 2.13-1.90 (m, 2H), 1.80-1.59 (m, 8H), 1.26-1.09 (m, 2H). Stereochemistry war arbitrarily assigned

### Example 225. Preparation of (I-61) (R)-3-(5-(4-((1-(4-((1S,2R)-6′-hydroxy-1′,2′,3′,4′,5,6,7,8-octahydro-[1,2′-binaphthalen]-1′-yl)phenyl)piperidin-4-yl)methyl)piperazin-1-yl)-1-oxoisoindolin-2-yl)piperidine-2,6-dione

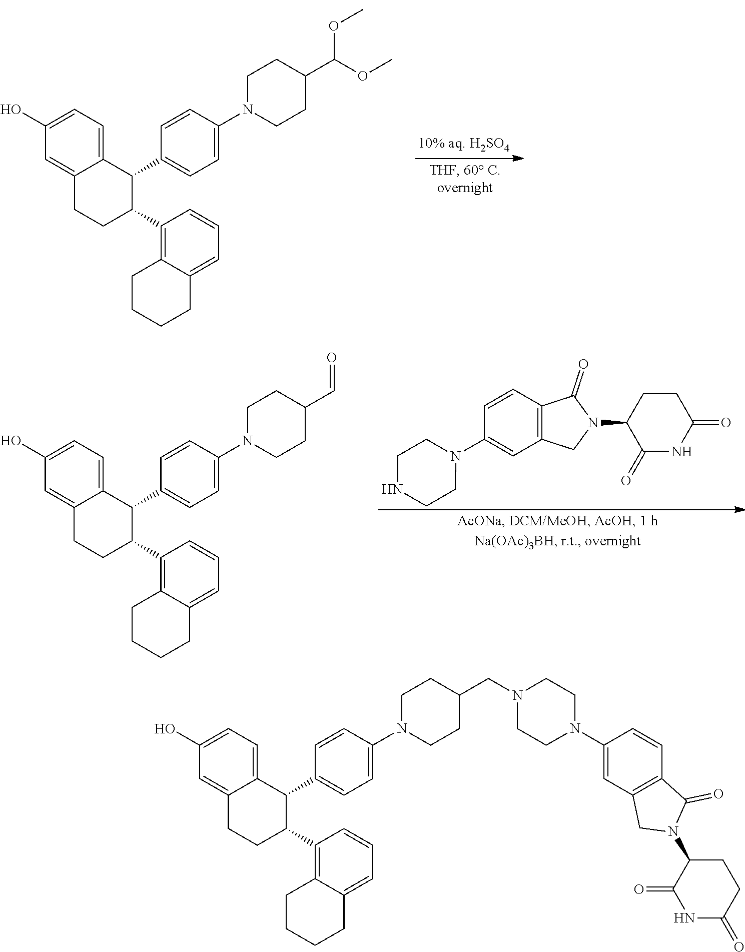

Step 1: A mixture of (1S,2R)-1′-(4-(4-(dimethoxymethyl)piperidin-1-yl)phenyl)-1′,2′,3′,4′,5,6,7,8-octahydro-[1,2′-binaphthalen]-6′-ol (150 mg, 1 Eq, 293 μmol) in 10% H₂SO₄ (1.5 mL) and THF (1.5 mL), then the mixture was stirred at 60° C. for 16 hour. The solvent was removed under vacuum. The residue was dissolved in water (20 mL), the aqueous solution was added NaHCO₃ to adjust pH=6 and washed by ethyl acetate (20 mL*2). The combined organic layer was dried over Na₂SO₄, filtered and concentrated under vacuum to give 1-(4-((1S,2R)-6′-hydroxy-1′,2′,3′,4′,5,6,7,8-octahydro-[1,2′-binaphthalen]-1′-yl)phenyl)piperidine-4-carbaldehyde (100 mg, 215 μmol, 73.3%) as a yellow oil. LC-MS (ESI+) m/z: 466.1 (M+H)+.

Step 2: A mixture of 1-(4-((1S,2R)-6′-hydroxy-1′,2′,3′,4′,5,6,7,8-octahydro-[1,2′-binaphthalen]-1′-yl)phenyl)piperidine-4-carbaldehyde (100 mg, 1 Eq, 215 μmol), (R)-3-(1-oxo-5-(piperazin-1-yl)isoindolin-2-yl)piperidine-2,6-dione, TsOH salt (140 mg, 1.3 Eq, 279 μmol) and Sodium acetate (88.1 mg, 5 Eq, 1.07 mmol) and Acetic acid (38.7 mg, 37.1 uL, 3 Eq, 644 μmol) in DCM (2 mL) and MeOH (2 mL) at 25° C. for 1 hour, then Sodium triacetoxyborohydride (91.0 mg, 2 Eq, 430 μmol) was added to the mixture and was stirred at 25° C. for 16 hour. The reaction was treated with H₂O (20 mL), extracted with EtOAc (20 mL*2). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 27%-57%, 7 min). The aqueous phase was lyophilized to dryness to give (R)-3-(5-(4-((1-(4-((1S,2R)-6′-hydroxy-1′,2′,3′,4′,5,6,7,8-octahydro-[1,2′-binaphthalen]-1′-yl)phenyl)piperidin-4-yl)methyl)piperazin-1-yl)-1-oxoisoindolin-2-yl)piperidine-2,6-dione (100.5 mg, 124.0 μmol, 57.7%, 95.974% Purity) as a white solid.

LC-MS (ESI+) m/z: 778.2 (M+H)⁺

HPLC: 95.974%, purity at 220 nm.

SFC: retention time, 3.095 min; Area, 89.696%;

¹H NMR (400 MHZ, DMSO-d₆) δ=11.01-10.92 (m, 1H), 9.21-9.01 (m, 1H), 8.19-8.12 (m, 0.383H), 7.56-7.48 (m, 1H), 7.11-7.00 (m, 2H), 6.86-6.78 (m, 1H), 6.73-6.62 (m, 2H), 6.61-6.57 (m, 1H), 6.56-6.50 (m, 2H), 6.50-6.45 (m, 1H), 6.19-6.12 (m, 2H), 6.00-5.93 (m, 1H), 5.09-5.01 (m, 1H), 4.37-4.29 (m, 1H), 4.25-4.17 (m, 1H), 4.16-4.10 (m, 1H), 3.55-3.48 (m, 2H), 3.48-3.27 (m, 11H), 2.98-2.83 (m, 5H), 2.81-2.66 (m, 2H), 2.57 (br s, 1H), 2.40-2.32 (m, 1H), 2.24-2.06 (m, 3H), 2.02-1.91 (m, 1H), 1.90-1.82 (m, 1H), 1.80-1.69 (m, 5H), 1.68-1.50 (m, 2H), 1.25-1.08 (m, 2H). Stereochemistry was arbitrarily assigned

### Example 226. Preparation of (I-58) (S)-3-(5-(4-((1-(4-((1S,2R)-2-(bicyclo[4.2.0]octa-1 (6), 2,4-trien-3-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

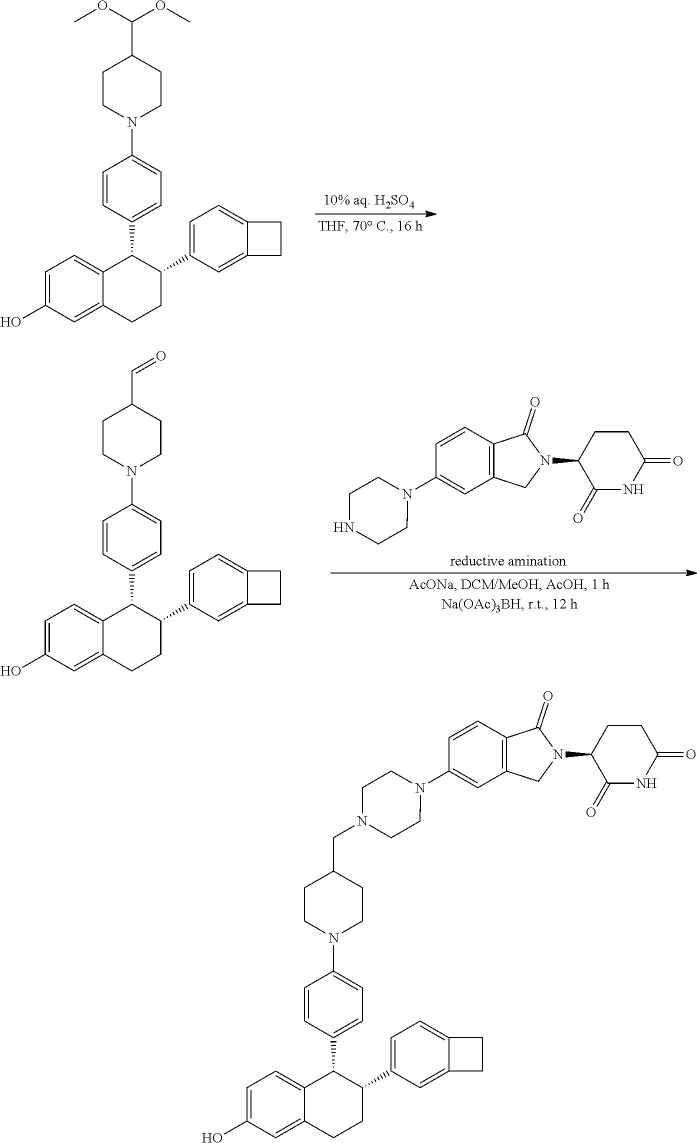

Step 1: A solution (5S,6R)-6-(bicyclo[4.2.0]octa-1,3,5-trien-3-yl)-5-(4-(4-(dimethoxymethyl)piperidin-1-yl)phenyl)-5,6,7,8-tetrahydronaphthalen-2-ol (98 mg, 1 Eq, 0.20 mmol) in 10% H₂SO₄ (7 mL) and THF (7 mL) was stirred at 70° C. for 16 hour to give a yellow suspension. The solvent was removed under vacuum. The aqueous solution was added NaHCO₃ to adjust pH=6. The residue was dissolved in water (20 mL) and extracted by ethyl acetate (50 mL). The combined organic layer was dried over Na₂SO₄, filtered and concentrated under vacuum to give 1-(4-((1S,2R)-2-(bicyclo[4.2.0]octa-1,3,5-trien-3-yl)-6-hydroxy-1,2,3,4-tetrahydronaphthalen-1-yl)phenyl)piperidine-4-carbaldehyde (80 mg, 0.18 mmol, 88%, 97.073% Purity) as a yellow oil. LC-MS (ESI⁺) m/z: 438.2 (M+H)⁺.

Step 2: To a solution of 1-(4-((1S,2R)-2-(bicyclo[4.2.0]octa-1,3,5-trien-3-yl)-6-hydroxy-1,2,3,4-tetrahydronaphthalen-1-yl)phenyl)piperidine-4-carbaldehyde (80 mg, 1 Eq, 0.18 mmol), (R)-3-(1-oxo-5-(piperazin-1-yl)isoindolin-2-yl)piperidine-2,6-dione, TsOH (91 mg, 1 Eq, 0.18 mmol) in DCM (3 mL), MeOH (3 mL) was added Sodium acetate (45 mg, 30 μL, 3 Eq, 0.55 mmol) was stirred at 25° C. for 60 mins, Then Sodium triacetoxyborohydride (77 mg, 54 μL, 2 Eq, 0.37 mmol) and acetic acid (33 mg, 32 μL, 3 Eq, 0.55 mmol) was added. The mixture was stirred at 25° C. for 16 hour. The reaction mixture was concentrated under reduced pressure to remove solvent. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep-HPLC (column: Welch Xtimate C18 150*30 mm*5 um; mobile phase: [water(FA)-ACN]; B % 25%-55%, 7 min) to give (S)-3-(5-(4-((1-(4-((1S,2R)-2-(bicyclo[4.2.0]octa-1 (6), 2,4-trien-3-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione was obtained as a white solid. LC-MS (ESI⁺) m/z: 750.2 (M+H)⁺.

LCMS: calc. for C₄₇H₅₁N₅O₄: 749.94, found: [M+H]⁺ 750.2.

HPLC: 99.850% purity at 220 nm.

¹H NMR (400 MHZ, DMSO-d₆) δ=10.99-10.92 (m, 1H), 8.20-8.15 (m, 0.551H), 7.55-7.49 (m, 1H), 7.10-7.02 (m, 2H), 6.88-6.82 (m, 1H), 6.69-6.62 (m, 2H), 6.61-6.54 (m, 3H), 6.51-6.46 (m, 2H), 6.27-6.19 (m, 2H), 5.11-5.01 (m, 1H), 4.38-4.28 (m, 1H), 4.26-4.15 (m, 1H), 4.12-4.05 (m, 1H), 3.59-3.48 (m, 3H), 3.46-3.34 (m, 6H), 3.11-2.84 (m, 8H), 2.71-2.58 (m, 1H), 2.46-2.43 (m, 2H), 2.41-2.29 (m, 2H), 2.23-2.17 (m, 2H), 2.14-1.89 (m, 3H), 1.81-1.73 (m, 2H), 1.69-1.60 (m, 2H), 1.25-1.11 (m, 2H)

SFC: retention time, 3.832 min; Area, 94.215%; Stereochemistry was arbitrarily assigned.

### Example 227. Preparation of (I-101) (R)-3-(5-(4-((1-(4-((1S,2R)-2-(bicyclo[4.2.0]octa-1,3,5-trien-2-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

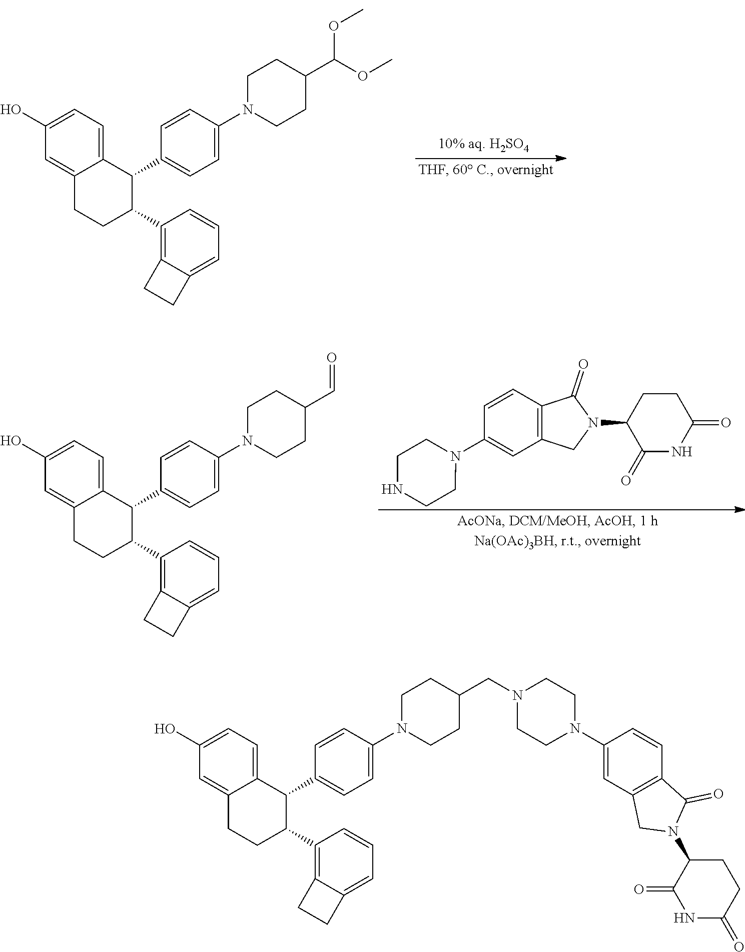

Step 1: A mixture of (5S,6R)-6-(bicyclo[4.2.0]octa-1,3,5-trien-2-yl)-5-(4-(4-(dimethoxymethyl)piperidin-1-yl)phenyl)-5,6,7,8-tetrahydronaphthalen-2-ol (55 mg, 1 Eq, 0.11 mmol) in 10% H₂SO₄ (1.5 mL) and THF (1.5 mL), then the mixture was stirred at 60° C. for 16 hour. The solvent was removed under vacuum. The residue was dissolved in water (20 mL), the aqueous solution was added NaHCO₃ to adjust pH=6. and extracted by ethyl acetate (20 mL*2). The combined organic layer was dried over Na₂SO₄, filtered and concentrated under vacuum to give 1-(4-((1S,2R)-2-(bicyclo[4.2.0]octa-1,3,5-trien-2-yl)-6-hydroxy-1,2,3,4-tetrahydronaphthalen-1-yl)phenyl)piperidine-4-carbaldehyde (50 mg, 0.11 mmol, 100%) as a yellow oil. LC-MS (ESI+) m/z: 438.1 (M+H)+.

Step 2: A mixture of 1-(4-((1S,2R)-2-(bicyclo[4.2.0]octa-1,3,5-trien-2-yl)-6-hydroxy-1,2,3,4-tetrahydronaphthalen-1-yl)phenyl)piperidine-4-carbaldehyde (50 mg, 1 Eq, 0.11 mmol), (R)-3-(1-oxo-5-(piperazin-1-yl)isoindolin-2-yl)piperidine-2,6-dione, TsOH salt (74 mg, 1.3 Eq, 0.15 mmol) and Sodium acetate (47 mg, 5 Eq, 0.57 mmol) and Acetic acid (21 mg, 20 μL, 3 Eq, 0.34 mmol) in DCM (2 mL) and MeOH (2 mL) at 25° C. for 1 hour, then Sodium triacetoxyborohydride (48 mg, 2 Eq, 0.23 mmol) was added to the mixture and was stirred at 25° C. for 16 hour. The reaction was treated with H₂O (20 mL), extracted with EtOAc (20 mL*2). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 26%-46%, 7 min). The aqueous phase was lyophilized to dryness to give (R)-3-(5-(4-((1-(4-((1S,2R)-2-(bicyclo[4.2.0]octa-1,3,5-trien-2-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (35.3 mg, 46.2 μmol, 40% yield, 98.192% Purity) as a white solid.

LC-MS (ESI+) m/z: 750.2 (M+H)⁺

HPLC: 98.192%, purity at 220 nm.

SFC: retention time, 3.045 min; Area, 88.894%; Stereochemistry was arbitrarily assigned.

¹H NMR (400 MHZ, DMSO-d₆) δ=11.04-10.82 (m, 1H), 8.23-8.18 (m, 0.584H), 7.56-7.47 (m, 1H), 7.11-7.01 (m, 3H), 6.95-6.88 (m, 1H), 6.86-6.80 (m, 1H), 6.71-6.63 (m, 1H), 6.62-6.54 (m, 3H), 6.52-6.46 (m, 1H), 6.22-6.13 (m, 2H), 5.10-5.01 (m, 1H), 4.38-4.29 (m, 1H), 4.25-4.16 (m, 1H), 4.08-4.01 (m, 1H), 3.61-3.49 (m, 4H), 3.28 (br s, 5H), 3.23-3.14 (m, 2H), 2.97-2.84 (m, 4H), 2.74-2.66 (m, 2H), 2.63-2.53 (m, 3H), 2.37-2.31 (m, 1H), 2.23-2.15 (m, 2H), 2.08-1.92 (m, 2H), 1.79-1.62 (m, 5H), 1.26-1.10 (m, 2H).

### Example 228. Preparation of (I-114) (35)-3-[5-[4-[[1-[4-[(1S, 2R)-6-hydroxy-2-(2-pyridyl)tetralin-1-yl]phenyl]-4-piperidyl]methyl]piperazin-1-yl]-1-oxo-isoindolin-2-yl]piperidine-2,6-dione and (I-115) (35)-3-15-[4-[[1-[4-[(1R,2S)-6-hydroxy-2-(2-pyridyl)tetralin-1-yl]phenyl]-4-piperidyl]methyl]piperazin-1-yl]-1-oxo-isoindolin-2-yl]piperidine-2,6-dione

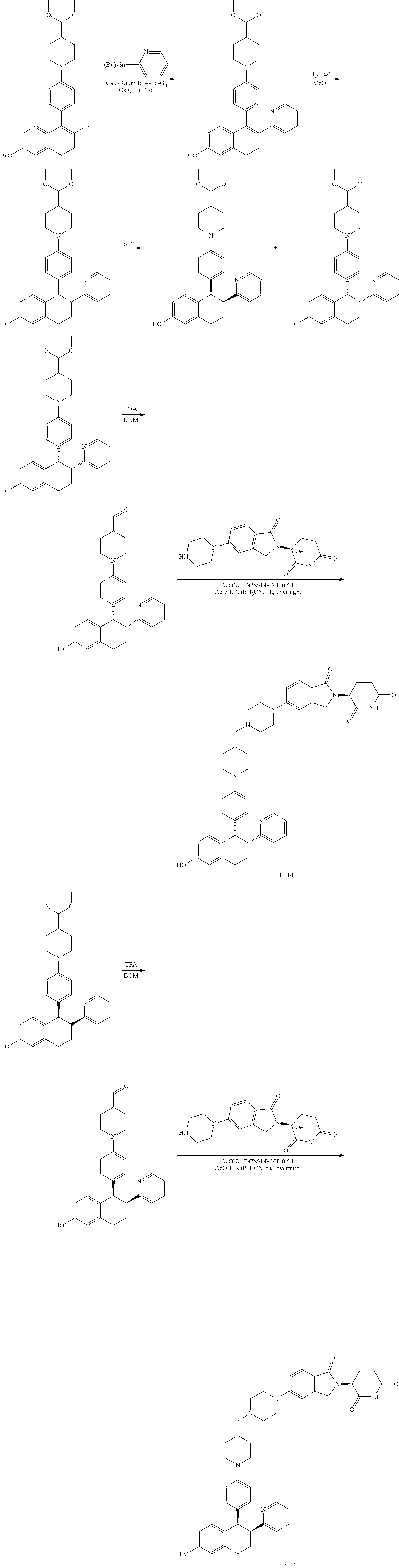

Step 1: To a solution of 1-[4-(6-benzyloxy-2-bromo-3,4-dihydronaphthalen-1-yl)phenyl]-4-(dimethoxymethyl)piperidine (1 g, 1.82 mmol, N/A purity, 1 eq) and tributyl(2-pyridyl)stannane (671.17 mg, 1.82 mmol, 1 eq) in toluene (10 mL) was added [2-(2-aminophenyl)phenyl]palladium (1+);bis(1-adamantyl)-butyl-phosphane;methanesulfonate (132.77 mg, 182.31 μmol, 0.1 eq), CuI (34.72 mg, 182.3 μmol, 0.1 eq) and CsF (553.88 mg, 3.65 mmol, 134.44 uL, 2 eq). The mixture was stirred at 95° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue. The residue was purified by flash silica gel chromatography (from pure PE to PE/EtOAc=3/1, TLC: PE/EtOAc=3/1, R_{f}=0.49) to yield 2-[6-benzyloxy-1-[4-[4-(dimethoxymethyl)-1-piperidyl]phenyl]-3,4-dihydronaphthalen-2-yl]pyridine (800 mg, 1.26 mmol, 69.0% yield, 86.0% purity) as green oil. LC-MS (ESI⁺) m/z 547.4 [M+H]⁺.

¹H NMR (400 MHz, CDCl₃)₂=8.55 (s, 1H), 7.46-7.32 (m, 6H), 6.96 (d, J=8.2 Hz, 3H), 6.89-6.73 (m, 5H), 6.68 (dd, J=2.6, 8.6 Hz, 1H), 5.08 (s, 2H), 4.10 (t, J=3.6 Hz, 1H), 3.72 (br d, J=12.2 Hz, 2H), 3.39 (s, 6H), 2.95 (s, 4H), 2.68 (br t, J=11.4 Hz, 2H), 1.88 (d, J=12.5 Hz, 2H), 1.76 (s, 1H), 1.51-1.40 (m, 2H)

Step 2: To a solution of 2-[6-benzyloxy-1-[4-[4-(dimethoxymethyl)-1-piperidyl]phenyl]-3,4-dihydronaphthalen-2-yl]pyridine (750 mg, 1.18 mmol, 86% purity, 1 eq) in MeOH (3 mL) was added Pd/C (600 mg, 10% purity). The mixture was stirred under H₂ (15 Psi) at 25° C. for 1 h. The reaction mixture was filtered and concentrated to yield a residue. The residue was separated by chiral SFC (column: DAICEL CHIRALCEL OJ (250 mm*30 mm, 10 um); mobile phase: [0.1% NH₃H₂O ETOH]; B %: 25%-25%, min), followed by lyophilization to yield (1S,2R)-1-[4-[4-(dimethoxymethyl)-1-piperidyl]phenyl]-2-(2-pyridyl)tetralin-6-ol (220 mg, 470.14 μmol, 39.8% yield, 98.0% purity, SFC: Rt=2.638) as a red solid and (1R,2S)-1-[4-[4-(dimethoxymethyl)-1-piperidyl]phenyl]-2-(2-pyridyl)tetralin-6-ol (250 mg, 534.25 μmol, 45.3% yield, 98.0% purity, SFC: Rt=2.986) as a red solid. LC-MS (ESI⁺) m/z 459.4 [M+H]⁺

¹H NMR of (1S,2R)-1-[4-[4-(dimethoxymethyl)-1-piperidyl]phenyl]-2-(2-pyridyl)tetralin-6-ol: (500 MHz, CDCl₃) δ=8.57 (d, J=4.4 Hz, 1H), 7.39 (dt, J=1.7, 7.6 Hz, 1H), 7.09 (dd, J=5.1, 7.1 Hz, 1H), 6.78 (d, J=8.2 Hz, 1H), 6.71 (d, J=2.3 Hz, 1H), 6.61-6.54 (m, 3H), 6.50 (d, J=7.9 Hz, 1H), 6.27 (d, J=8.7 Hz, 2H), 4.45 (d, J=5.0 Hz, 1H), 4.06 (d, J=7.5 Hz, 1H), 3.57-3.50 (m, 3H), 3.35 (s, 6H), 3.09-2.93 (m, 2H), 2.52 (br t, J=11.4 Hz, 2H), 2.18 (dq, J=6.0, 12.8 Hz, 1H), 1.88 (dd, J=5.4, 12.4 Hz, 1H), 1.80 (br d, J=11.1 Hz, 2H), 1.73-1.65 (m, 2H), 1.40 (br d, J=11.9 Hz, 2H)

¹H NMR of (1R,2S)-1-[4-[4-(dimethoxymethyl)-1-piperidyl]phenyl]-2-(2-pyridyl)tetralin-6-ol: (500 MHz, CDCl₃)₂=8.57 (d, J=4.1 Hz, 1H), 7.41 (t, J=7.6 Hz, 1H), 7.13-7.08 (m, 1H), 6.76 (d, J=8.4 Hz, 1H), 6.71 (d, J=2.3 Hz, 1H), 6.59 (dd, J=2.6, 8.2 Hz, 3H), 6.51 (d, J=7.9 Hz, 1H), 6.28 (d, J=8.5 Hz, 2H), 4.46 (br d, J=5.0 Hz, 1H), 4.06 (d, J=7.3 Hz, 1H), 3.54 (br d, J=10.5 Hz, 3H), 3.36 (s, 6H), 3.08-3.01 (m, 1H), 3.00-2.91 (m, 1H), 2.55 (s, 2H), 2.17 (dq, J=6.0, 12.7 Hz, 1H), 1.87 (dd, J=5.6, 12.4 Hz, 1H), 1.81 (d, J=12.2 Hz, 2H), 1.69 (dt, J=3.9, 7.6 Hz, 2H), 1.43 (s, 2H)

Step 3: To a solution of (1S,2R)-1-[4-[4-(dimethoxymethyl)-1-piperidyl]phenyl]-2-(2-pyridyl)tetralin-6-ol (60 mg, 128.22 μmol, 98% purity, 1 eq) in DCM (2.5 mL) was added TFA (770.00 mg, 6.75 mmol, 0.5 mL, 52.67 eq). The mixture was stirred at 25° C. for 1 h. The reaction mixture was quenched by addition of water (30 mL), extracted with EtOAc (20 mL×3). The combined organic layers were washed with brine (10 mL), dried over Na₂SO₄, filtered and concentrated under reduced pressure to yield 1-[4-[(1S,2R)-6-hydroxy-2-(2-pyridyl)tetralin-1-yl]phenyl]piperidine-4-carbaldehyde (60 mg, 116.36 μmol, 90.7% yield, 80.0% purity) as a brown solid which was used in the next step without further purification. LC-MS (ESI⁺) m/z 413.3 [M+H]⁺.

Step 4: To a solution of 1-[4-[(1S,2R)-6-hydroxy-2-(2-pyridyl)tetralin-1-yl]phenyl]piperidine-4-carbaldehyde (60 mg, 116.36 μmol, 80% purity, 1 eq) and (35)-3-(1-oxo-5-piperazin-1-yl-isoindolin-2-yl)piperidine-2,6-dione (87.37 mg, 174.54 μmol, 1.5 eq, TsOH) in DCM (2.5 mL) and MeOH (2.5 mL), after stirred 30 min was added NaBH(OAc)₃ (80.00 mg, 377.46 μmol, 3.24 eq). The mixture was stirred at 25° C. for 12 h. The reaction mixture was concentrated to yield a residue. The residue was purified by preparative HPLC (column: Welch Xtimate C18 150*25 mm*5 um; mobile phase: [water (NH₄HCO₃)-ACN]; B %: 40%-55%, 13 min), followed by lyophilization to yield (3S)-3-[5-[4-[[1-[4-[(1S,2R)-6-hydroxy-2-(2-pyridyl)tetralin-1-yl]phenyl]-4-piperidyl]methyl]piperazin-1-yl]-1-oxo-isoindolin-2-yl]piperidine-2,6-dione (25.7 mg, 35.45 μmol, 30.47% yield, 100% purity) as a white solid. LC-MS (ESI⁺) m/z 725.5 [M+H]⁺.

LCMS: calc. for C₄₄H₄₈N₆O₄:724.37, found: [M+H]⁺ 725.5.

HPLC: 100% purity at 220 nm.

¹H NMR (500 MHz, CD₃OD) δ=8.45 (d, J=4.9 Hz, 1H), 7.63 (d, J=8.4 Hz, 1H), 7.51 (dt, J=1.8, 7.7 Hz, 1H), 7.20 (dd, J=5.4, 7.1 Hz, 1H), 7.12-7.04 (m, 2H), 6.72 (d, J=8.4 Hz, 1H), 6.68-6.64 (m, 2H), 6.61 (d, J=8.7 Hz, 2H), 6.53 (dd, J=2.5, 8.3 Hz, 1H), 6.28 (d, J=8.5 Hz, 2H), 5.09 (dd, J=5.2, 13.3 Hz, 1H), 4.43-4.35 (m, 2H), 3.54-3.47 (m, 3H), 3.40-3.33 (m, 5H), 3.10-2.95 (m, 2H), 2.94-2.84 (m, 1H), 2.81-2.74 (m, 1H), 2.64-2.52 (m, 6H), 2.46 (dq, J=4.8, 13.2 Hz, 1H), 2.32-2.22 (m, 3H), 2.18-2.11 (m, 1H), 1.91-1.83 (m, 3H), 1.75-1.64 (m, 1H), 1.34-1.27 (m, 2H).

Step 5: To a solution of (1R,2S)-1-[4-[4-(dimethoxymethyl)-1-piperidyl]phenyl]-2-(2-pyridyl)tetralin-6-ol (60 mg, 128.22 μmol, 98% purity, 1 eq) in DCM (2.5 mL) was added TFA (770.00 mg, 6.75 mmol, 0.5 mL, 52.67 eq). The mixture was stirred at 25° C. for 1 h. The reaction mixture was quenched by addition of water (30 mL), extracted with EtOAc (20 mL×3). The combined organic layers were washed with brine (10 mL), dried over Na₂SO₄, filtered and concentrated under reduced pressure to yield 1-[4-[(1R,2S)-6-hydroxy-2-(2-pyridyl)tetralin-1-yl]phenyl]piperidine-4-carbaldehyde (60 mg, 116.36 μmol, 90.75% yield, 80% purity) as a brown solid which was used in the next step without further purification. LC-MS (ESI⁺) m/z 413.3 [M+H]⁺.

Step 6: To a solution of 1-[4-[(1R,2S)-6-hydroxy-2-(2-pyridyl)tetralin-1-yl]phenyl]piperidine-4-carbaldehyde (60 mg, 116.36 μmol, 80% purity, 1 eq) and (35)-3-(1-oxo-5-piperazin-1-yl-isoindolin-2-yl)piperidine-2,6-dione (87.37 mg, 174.54 μmol, 1.5 eq, TsOH) in DCM (2.5 mL) and MeOH (2.5 mL), after stirred 30 min was added NaBH(OAc); (80.00 mg, 377.46 μmol, 3.24 eq). The mixture was stirred at 25° C. for 12 h. The residue was filtered and purified by preparative HPLC (column: Welch Xtimate C18 150*25 mm*5 um; mobile phase: [water (NH₄HCO₃)-ACN]; B %: 40%-55%, 13 min), followed by lyophilization to yield (3S)-3-[5-[4-[[1-[4-[(1R,2S)-6-hydroxy-2-(2-pyridyl)tetralin-1-yl]phenyl]-4-piperidyl]methyl]piperazin-1-yl]-1-oxo-isoindolin-2-yl]piperidine-2,6-dione (40.2 mg, 55.46 μmol, 47.66% yield, 100% purity) as a white solid. LC-MS (ESI⁺) m/z 713.5 [M+H]⁺.

LCMS: calc. for C44H48N4O5: 712.36, found: [M+H]⁺ 713.5.

HPLC: 100% purity at 220 nm.

¹H NMR (500 MHz, CD₃OD) δ=8.45 (d, J=4.1 Hz, 1H), 7.63 (d, J=8.5 Hz, 1H), 7.51 (dt, J=1.8, 7.7 Hz, 1H), 7.20 (dd, J=5.2, 6.7 Hz, 1H), 7.11-7.04 (m, 2H), 6.71 (d, J=8.4 Hz, 1H), 6.68-6.63 (m, 2H), 6.61 (d. J=8.7 Hz, 2H), 6.53 (dd, J=2.5, 8.3 Hz, 1H), 6.28 (d, J=8.7 Hz, 2H), 5.09 (dd, J=5.2, 13.4 Hz, 1H), 4.43-4.32 (m, 2H), 3.55-3.46 (m, 3H), 3.40-3.32 (m, 5H), 3.10-2.95 (m, 2H), 2.95-2.84 (m, 1H), 2.81-2.74 (m, 1H), 2.65-2.51 (m, 6H), 2.46 (dq, J=4.7, 13.2 Hz, 1H), 2.33-2.21 (m, 3H), 2.18-2.11 (m, 1H), 1.93-1.80 (m, 3H), 1.69 (br s, 1H), 1.30 (q, J=11.8 Hz, 2H)

### Example 229. Preparation of (I-107) (R)-3-(5-(4-((1-(4-((1R,2S)-6-hydroxy-2-(pyridine-3-yl)-1,2,3,4-tetrahydronaphthalen-1-yl)phenyl)piperidin-4-yl)methyl)piperazin-1-yl)-1-oxoisoindolin-2-yl)piperidine-2,6-dione

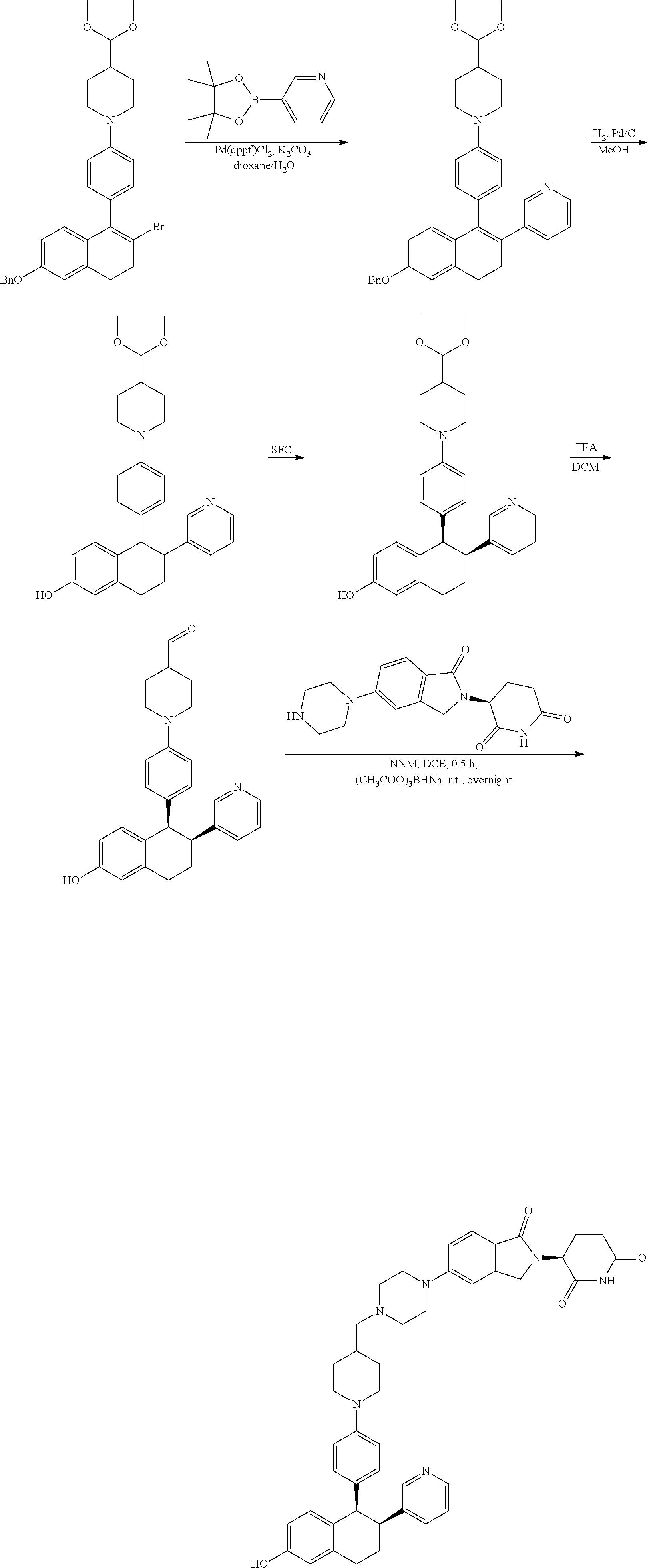

Step 1: A mixture of starting aryl bromide (1.50 g, 1 eq., 2.73 mmol), pyridin-3-ylboronic acid (336 mg, 1 eq., 2.73 mmol), sodium carbonate (290 mg, 1 eq., 2.73 mmol) and Reactant IV (2.00 g, 1 eq., 2.73 mmol) in Dioxane (15 mL) and water (3 mL) was degassed and purged with N₂ for 3 times, and then the mixture was stirred at 80° C. for 4 hour under N₂ atmosphere. LCMS showed the reaction was completed. The reaction mixture was quenched by addition EA 30 mL at 20° C., and then diluted with water 30 mL and extracted with EA 90 mL (30 mL*3). The combined organic layers were washed with EA 30 mL (10 mL*3), dried over Na₂SO₄, filtered and concentrated under reduced pressure to give a residue. The crude material was purified on silica gel column chromatography (from PE/EtOAc=I/O to 1/1) to yield 3-(6-(benzyloxy)-1-(4-(4-(dimethoxymethyl)piperidin-1-yl)phenyl)-3,4-dihydronaphthalen-2-yl)pyridine (1.3 g, 2.4 mmol, 87%) as a yellow solid

LC-MS (ESI⁺) m/z: 547.4 (M+H)⁺.

¹H NMR (500 MHz, DMSO-d₆) δ: ppm 8.21 (dd, J=4.88, 1.53 Hz, 1H), 8.16 (d, J=1.68 Hz, 1H), 7.36-7.47 (m, 5H), 7.30-7.35 (m, 1H), 7.17 (dd, J=7.93, 4.73 Hz, 1H), 6.94 (d, J=2.59 Hz, 1H), 6.81 (s, 4H), 6.74 (dd, J=8.62, 2.67 Hz, 1H), 6.60 (d, J=8.70 Hz, 1H), 5.09 (s, 2H), 4.08 (d, J=6.56 Hz, 1H), 3.68 (br d, J=12.21 Hz, 2H), 3.27 (s, 6H), 2.87-2.94 (m, 2H), 2.66-2.74 (m, 2H), 2.54-2.62 (m, 2H), 1.65-1.74 (m, 3H), 1.23-1.35 (m, 2H).

Step 2: To a solution of 3-(6-(benzyloxy)-1-(4-(4-(dimethoxymethyl)piperidin-1-yl)phenyl)-3,4-dihydronaphthalen-2-yl)pyridine (0.50 g, 1 eq., 0.91 mmol) in EA (15 mL) was added Pd/C (10%, 0.5 g) under N₂ atmosphere. The suspension was degassed and purged with H2 for 3 times. The mixture was stirred under H₂ at 60° C. for 24 h. LCMS showed the reaction was completed. The crude was purified by prep. HPLC together (Waters Xbridge BEH C18 100*30 mm*10 um, water (NH₃H₂O+NH₄HCO₃)-CAN as a mobile phase, from 40% to 70%, Gradient Time (min): 11, Flow Rate (ml/min): 50) to give 5-(4-(4-(dimethoxymethyl)piperidin-1-yl)phenyl)-6-(pyridine-3-yl)-5,6,7,8-tetrahydronaphthalen-2-ol (210 mg, 458 μmol, 50%) as a white solid. LC-MS (ESI⁺) m/z: 459.2 (M+H)⁺.

Step 3: 5-(4-(4-(dimethoxymethyl)piperidin-1-yl)phenyl)-6-(pyridine-3-yl)-5,6,7,8-tetrahydronaphthalen-2-ol was purified by SFC(Column: DAICEL CHIRALPAK AS (250 mm*30 mm, 10 um)); Mobile phase: 0.1% NH₃H₂O ETOH; from 30% to 30%; Flow rate: 90 mL/min) to give (5R,6S)-5-(4-(4-(dimethoxymethyl)piperidin-1-yl)phenyl)-6-(pyridin-3-yl)-5,6,7,8-tetrahydronaphthalen-2-ol (75 mg, 0.16 mmol, 36%) as white solid. LC-MS (ESI⁺) m/z: 459.3 (M+H)⁺.

Step 4: To a solution of (5R, 6S)-5-(4-(4-(dimethoxymethyl)piperidin-1-yl)phenyl)-6-(pyridin-3-yl)-5,6,7,8-tetrahydronaphthalen-2-ol (70 mg, 1 eq., 0.15 mmol) in DCM (2 mL) was added TFA (52 mg, 35 μL, 3 eq., 0.46 mmol). The mixture was stirred at 20° C. for 4 hour. LCMS showed the reaction was completed. The mixture was concentrated in vacuo to give crude product. No further purification as it is used for the next step directly. LC-MS (ESI+) m/z: 413.2 (M+H)+.

Step 5: To a solution of 1-(4-((1R,2S)-6-hydroxy-2-(pyridin-3-yl)-1,2,3,4-tetrahydronaphthalen-1-yl)phenyl)piperidine-4-carbaldehyde (30.0 mg, 1 eq., 72.7 μmol) and (R)-3-(1-oxo-5-(piperazin-1-yl)isoindolin-2-yl)piperidine-2,6-dione (35.8 mg, 1.5 eq., 109 μmol) in DCE (3 mL) was added dropwise 4-methylmorpholine (7.36 mg, 1 eq., 72.7 μmol) to pH=8 at 20° C.

After addition, the mixture was stirred at this temperature for 0.5 hour, and then sodium triacetoxyhydroborate (30.8 mg, 2 eq., 145 μmol) was added dropwise at 20° C. The resulting mixture was stirred at 20° C. for 3 hour. LCMS showed the reaction was completed. The crude was purified by prep. HPLC together (Welch Xtimate C18 150*25 mm*5 um, water (NH₃H₂O+NH₄HCO₃)-ACN as a mobile phase, from 35% to 65%, Gradient Time (min): 11, Flow Rate (ml/min): 25) to give (R)-3-(5-(4-((1-(4-((1R,2S)-6-hydroxy-2-(pyridine-3-yl)-1,2,3,4-tetrahydronaphthalen-1-yl)phenyl)piperidin-4-yl)methyl)piperazin-1-yl)-1-oxoisoindolin-2-yl)piperidine-2,6-dione (27.4 mg, 37.8 μmol, 52.0%) as a white solid.

LC-MS (ESI+) m/z: 725.3 (M+H)+

¹H NMR (500 MHz, METHANOL-d₄) δ: ppm 8.15-8.48 (m, 1H), 7.78-8.11 (m, 1H), 7.64 (d. J=8.39 Hz, 1H), 7.21 (br s, 2H), 7.04-7.13 (m, 2H), 6.62-6.74 (m, 4H), 6.53 (dd, J=8.39, 2.59 Hz, 1H), 6.36 (d, J=8.70 Hz, 2H), 5.09 (dd, J=13.35, 5.11 Hz, 1H), 4.33-4.53 (m, 2H), 4.20 (d, J=5.19 Hz, 1H), 3.51-3.57 (m, 2H), 3.37-3.44 (m, 5H), 2.99-3.09 (m, 2H), 2.85-2.93 (m, 1H), 2.66-2.81 (m, 5H), 2.56-2.63 (m, 2H), 2.43-2.52 (m, 1H), 2.40 (br d, J=6.87 Hz, 2H), 2.10-2.19 (m, 1H), 1.87 (br d, J=12.97 Hz, 2H), 1.70-1.81 (m, 2H), 1.23-1.41 (m, 3H). Stereochemistry was arbitrarily assigned

### Example 230. Preparation of (I-132) (S)-3-(5-(4-((1-(4-((1R,2S)-6-hydroxy-2-(pyridin-4-yl)-1,2,3,4-tetrahydronaphthalen-1-yl)phenyl)piperidin-4-yl)methyl)piperazin-1-yl)-1-oxoisoindolin-2-yl)piperidine-2,6-dione

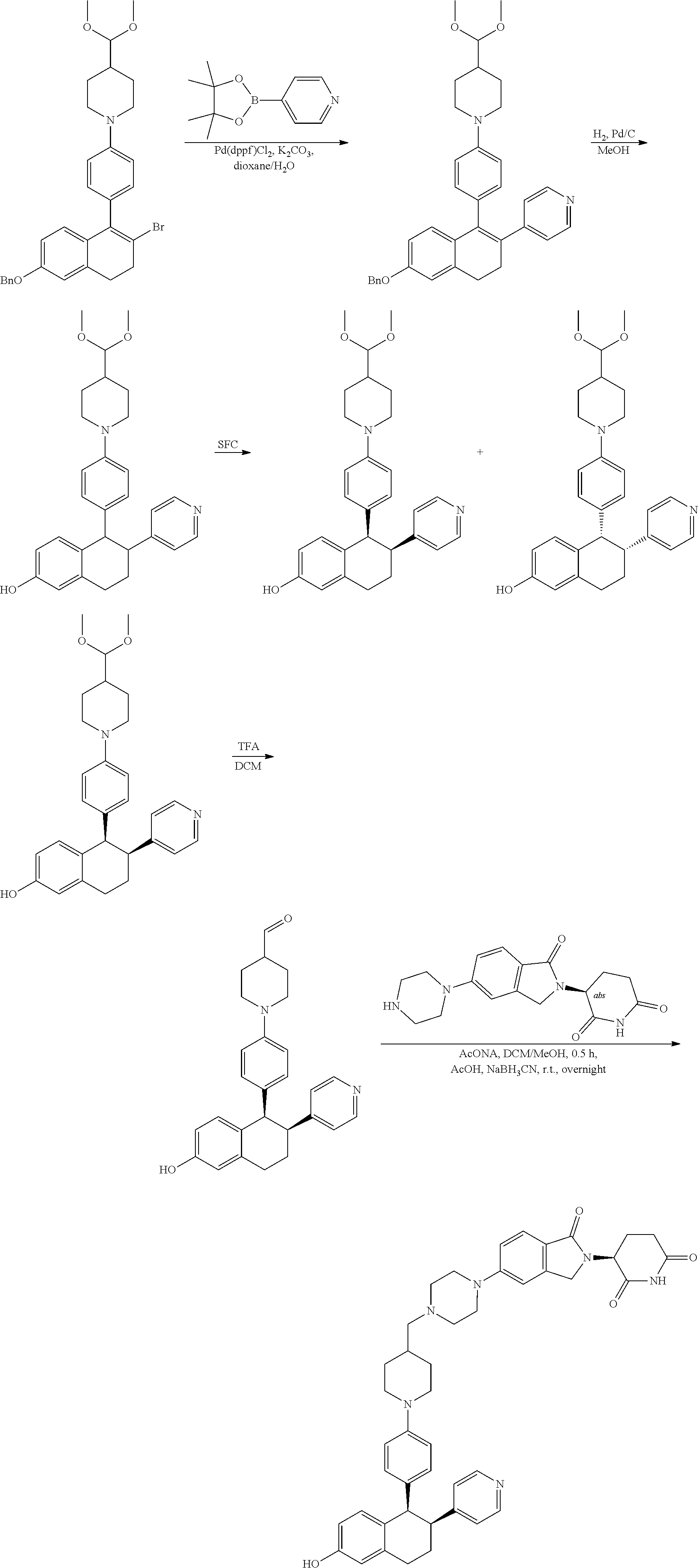

Step 1: To a mixture of 1-(4-(6-(benzyloxy)-2-bromo-3,4-dihydronaphthalen-1-yl)phenyl)-4-(dimethoxymethyl)piperidine (3.00 g, 1 Eq, 5.47 mmol), 4-(4,4,5,5-tetramethyl-1,3,2-dioxaborolan-2-yl)pyridine (1.12 g, 1 Eq, 5.47 mmol), PdCl₂(dppf) (800 mg, 0.2 Eq, 1.09 mmol) and K₂CO₃ (2.27 g, 3 Eq, 16.4 mmol) in Dioxane (100 mL) and H₂O (2 mL) was added reagent in one portion at 25° C. under N₂. The mixture was Suction air for 3 times under N₂, then heated to 85° C. and stirred for 8 hours. TLC showed the reaction was completed. The mixture was cooled to 20° C., solid was filtered and concentrated in reduced pressure at 25° C. The residue was poured into ice-water (w/w=1/1) (200 mL) and stirred for 5 min. The aqueous phase was extracted with ethyl acetate (100 mL*2). The combined organic phase was washed with brine (50 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=5/1, N/1) to afford 5-(6-(benzyloxy)-2-(pyridin-4-yl)-3,4-dihydronaphthalen-1-yl)-2-(4-(dimethoxymethyl)piperidin-1-yl)benzene-1-ylium (2.54 g, 4.6 mmol, 84%, 99% Purity) as yellow solid.

Step 2: To a mixture of 4-(6-(benzyloxy)-1-(4-(4-(dimethoxymethyl)piperidin-1-yl)phenyl)-3,4-dihydronaphthalen-2-yl)pyridine (2.25 g, 1 Eq, 4.12 mmol) and P d/C (438 mg, 1 Eq, 4.12 mmol) in Ethyl acetate (100 mL) was added reagent in one portion at 25° C. under H₂. The mixture was stirred at 25° C. for 5 min, then heated to 80° C. and stirred for 24 hours at 50 psi. TLC showed the reaction was completed. The mixture was cooled to 20° C., add 200 ml solution (DCM and MeOH1:1) to the mixture and filer solid and concentrated in reduced pressure at 30° C. to afford 5-(4-(4-(dimethoxymethyl)piperidin-1-yl)phenyl)-6-(pyridine-4-yl)-5,6,7,8-tetrahydronaphthalen-2-ol (1.68 g, 3.88 mmol, 94.26%) as yellow solid.

Step 3: To a mixture of 5-(4-(4-(dimethoxymethyl)piperidin-1-yl)phenyl)-6-(pyridin-4-yl)-5,6,7,8-tetrahydronaphthalen-2-ol (1.00 g, 1 Eq. 2.18 mmol) in 5-(4-(4-(dimethoxymethyl)piperidin-1-yl)phenyl)-6-(pyridin-4-yl)-5,6,7,8-tetrahydronaphthalen-2-ol (1.00 g, 1 Eq, 2.18 mmol). SFC to give (5R,6S)-5-(4-(4-(dimethoxymethyl)piperidin-1-yl)phenyl)-6-(pyridin-4-yl)-5,6,7,8-tetrahydronaphthalen-2-ol (350 mg, 763 μmol, 70.0%) and as yellow solid.

Step 4: To a mixture of (5R,6S)-5-(4-(4-(dimethoxymethyl)piperidin-1-yl)phenyl)-6-(pyridine-4-yl)-5,6,7,8-tetrahydronaphthalen-2-ol (350 mg, 1 Eq, 763 μmol) and TFA (435 mg, 294 μL, 5 Eq, 3.82 mmol) in DCM (20 mL) was added reagent in one portion at 25° C. . . . The mixture was stirred at 25° C. for 2 hours. TLC showed the reaction was completed. The mixture was concentrated in reduced pressure at 25° C. to provide 1-(4-((1R,2S)-6-hydroxy-2-(pyridine-4-yl)-1,2,3,4-tetrahydronaphthalen-1-yl)phenyl)piperidine-4-carbaldehyde (298 mg, 0.48 mmol, 62%, 66% Purity) The residue was used to next step.

Step 5: To a mixture of 1-(4-((1R,2S)-6-hydroxy-2-(pyridine-4-yl)-1,2,3,4-1411yridine 1411onaphthalene-1-yl)phenyl)piperidine-4-carbaldehyde (100 mg, 1 Eq, 242 μmol), (S)-3-(1-oxo-5-(piperazin-1-yl)isoindolin-2-yl)piperidine-2,6-dione (159 mg, 2 Eq, 485 μmol) in DCM (15 mL) was added reagent in one portion at 25° C. The mixture was stirred at 25° C. for 5 min, then add 4-methylmorfolin (49.0 mg, 53 μL, 2 Eq, 485 μmol) and Sodium triacetoxyborohydride (154 mg, 108 μL, 3 Eq, 727 μmol) to adjust PH to about 6 and stirred for 3 hours. TLC showed the reaction was completed. The mixture was reduced pressure at 25° C. The residue was further purified by pre-HPLC to provide (S)-3-(5-(4-((1-(4-((1R,2S)-6-hydroxy-2-(pyridine-4-yl)-1,2,3,4-tetrahydronaphthalen-1-yl)phenyl)piperidin-4-yl)methyl)piperazin-1-yl)-1-oxoisoindolin-2-yl)piperidine-2,6-dione (54.8 mg, 75.6 μmol, 31.2%, 100% Purity) as white solid. The white solid was re-crystallized from water, dried by lyophilization to give a white solid.

### Example 231. Preparation of (I-59) (3S)-3-[5-[4-[[1-[4-[(1R,2S)-6-hydroxy-2-tetralin-6-yl-tetralin-1-yl]phenyl]-4-piperidyl]methyl]piperazin-1-yl]-1-oxo-isoindolin-2-yl]piperidine-2,6-dione

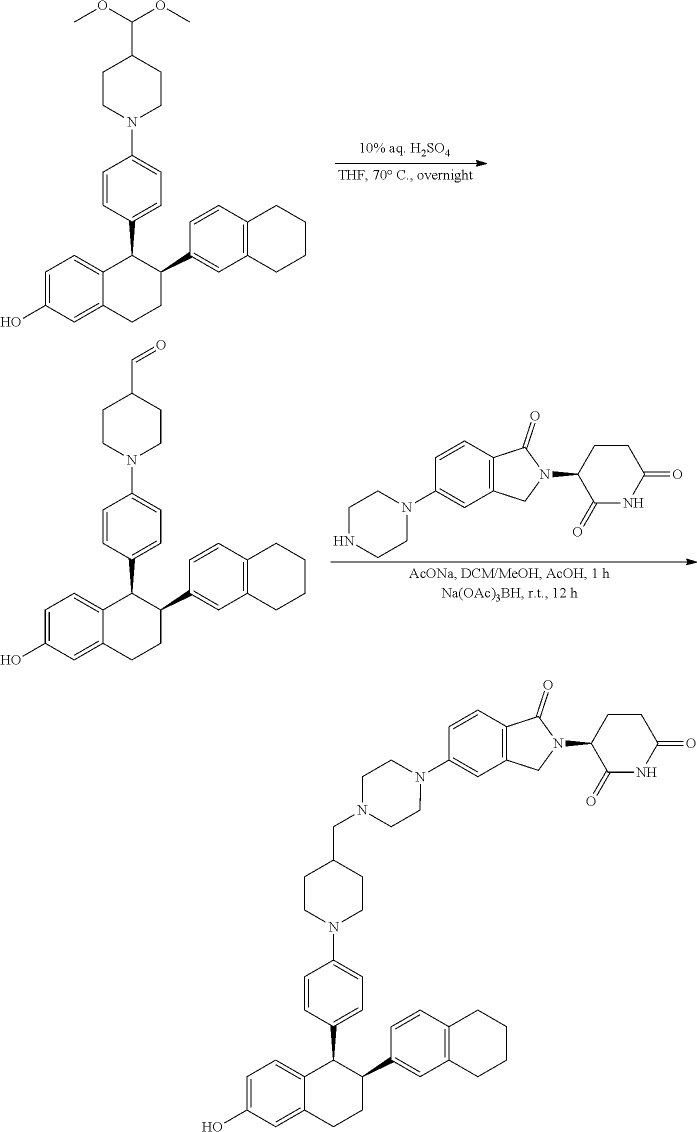

Step 1: To a solution of (1R,2S)-1-[4-[4-(dimethoxymethyl)-1-piperidyl]phenyl]-2-tetralin-6-yl-tetralin-6-ol (202 mg, 394.77 mmol, 1 eq) was added H₂SO₄ (10 mL, 10% purity) and THF (10 mL). The mixture was stirred at 70° C. for 12 h. LCMS showed 78% of desired compound was detected. The reaction mixture was quenched by addition NaHCO₃30 mL at 25° C., and then diluted with H₂O 20 mL and extracted with Ethyl acetate (50 mL*3). The combined organic layers were washed with brine (50 mL), dried over Na₂SO₄, filtered and concentrated under reduced pressure to give the compound as a yellow oil. The crude product 1-[4-[(1R, 2S)-6-hydroxy-2-tetralin-6-yl-tetralin-1-yl]phenyl]piperidine-4-carbaldehyde (180 mg, 386.58 μmol, 90% yield) was used into the next step without further purification. LC-MS (ESI⁺) m/z: 484.2 (M+H2O)⁺.

Step 2: A mixture of 1-[4-[(1R,2S)-6-hydroxy-2-tetralin-6-yl-tetralin-1-yl]phenyl]piperidine-4-carbaldehyde (180 mg, 386.58 μmol, 1 eq), HOAc (69.64 mg, 1.16 mmol, 66.33 uL, 3 eq), NaOAc (95.14 mg, 1.16 mmol, 3 eq) and (3S)-3-(1-oxo-5-piperazin-1-yl-isoindolin-2-yl)piperidine-2,6-dione (193.51 mg, 386.58 μmol, 1 eq, p-TSA salt) in DCM (4 mL) and MeOH (4 mL) was stirred at 25° C. for 1 h, Na(OAc)₃BH (163.86 mg, 773.16 μmol, 2 eq) was added to the reaction and then the mixture was stirred at 25° C. for 16 hr. The reaction mixture was quenched by addition 50 mL H2O, and then diluted with 50 mL H₂O and extracted with Ethyl acetate (50 mL*2). The organic layers were dried with anhydrous Na₂SO₄, filtered, and concentrated to give the crude product. The residue was purified by prep-HPLC (column: Welch Xtimate C18 150*30 mm*5 um; mobile phase: [water(FA)-ACN]; B %: 32%-62%, 7 min). Compound (3S)-3-[5-[4-[[1-[4-[(1R,2S)-6-hydroxy-2-tetralin-6-yl-tetralin-1-yl]phenyl]-4-piperidyl]methyl]piperazin-1-yl]-1-oxo-isoindolin-2-yl]piperidine-2,6-dione (37 mg, 47.56 μmol, 12.30% yield) was obtained as a yellow solid.

LC-MS (ESI+) m/z: 778.2 (M+H)+

HPLC: 98.731% purity at 220 nm.

SFC: Rₜ: 3.206 min; Area, 91.573%; Stereochemistry was arbitrarily assigned.

¹H NMR (400 MHZ, DMSO-d₆) δ=10.99-10.92 (m, 1H), 9.14-9.06 (m, 1H), 8.18-8.09 (m, 0.497H), 7.57-7.50 (m, 1H), 7.13-7.02 (m, 2H), 6.85-6.77 (m, 1H), 6.67-6.62 (m, 1H), 6.62-6.55 (m, 3H), 6.53-6.45 (m, 2H), 6.39 (s, 1H), 6.25 (br d, J=8.5 Hz, 2H), 5.05 (dd, J=5.0, 13.3 Hz, 1H), 4.37-4.30 (m, 1H), 4.25-4.18 (m, 1H), 4.08-4.03 (m, 1H), 3.60-3.46 (m, 3H), 3.22-3.11 (m, 2H), 3.02-2.81 (m, 4H), 2.57 (br s, 9H), 2.43-2.28 (m, 4H), 2.15-1.91 (m, 3H), 1.80-1.60 (m, 9H), 1.25-1.11 (m, 2H).

### Example 232. Preparation of (I-62) (R)-3-(5-(4-((1-(4-((1R,2S)-6′-hydroxy-1′,2′,3′,4′,5,6,7,8-octahydro-[1,2′-binaphthalen]-1′-yl)phenyl)piperidin-4-yl)methyl)piperazin-1-yl)-1-oxoisoindolin-2-yl)piperidine-2,6-dione

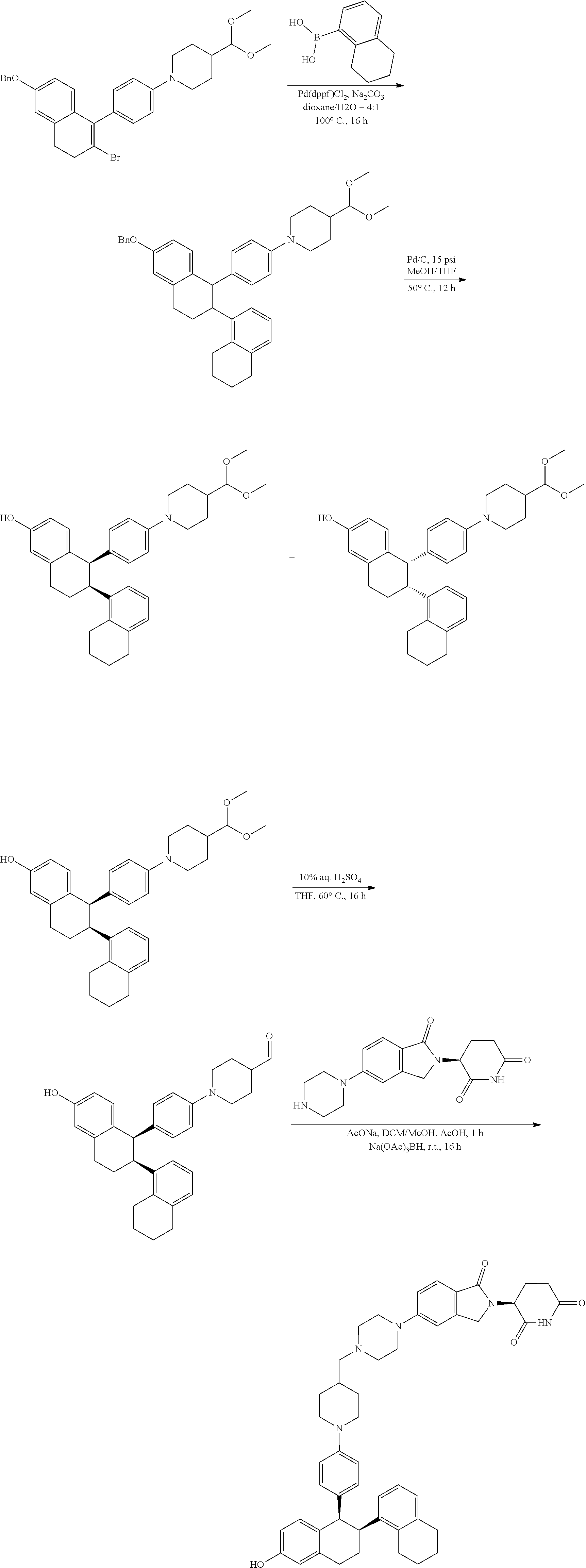

Step 1: A mixture of 1-(4-(6-(benzyloxy)-2-bromo-3,4-dihydronaphthalen-1-yl)phenyl)-4-(dimethoxymethyl)piperidine (500 mg, 1 Eq, 912 μmol), 5,6,7,8-Tetrahydronaphthalen-1-ylboronicacid (193 mg, 1.2 Eq, 1.09 mmol), Na₂CO₃ (290 mg, 3 Eq, 2.73 mmol) and [1,1′-Bis(diphenylphosphino)ferrocene]dichloropalladium(II) Complex With Dichloromethane (74.4 mg, 0.1 Eq, 91.2 μmol) in Dioxane (8 mL) and H₂O (2 mL) at r.t. N₂ was bubbled into the mixture for 5 min. The reaction mixture was heated at 100° C. for 16 hour. Then the reaction was cooled to room temperature. The mixture was treated with H₂O (100 mL), extracted with EtOAc (100 mL*2). The combined extracts was dried over anhydrous Na₂SO₄, filtered and concentrated to dryness in vacuo to give a residue. The residue was subjected to column chromatography over silica gel (gradient elution: 0˜20% ethyl acetate in petroleum ether). The pure fractions were collected and concentrated to dryness in vacuo to give 1-(4-(6′-(benzyloxy)-3′,4′,5,6,7,8-hexahydro-[1,2′-binaphthalen]-1′-yl)phenyl)-4-(dimethoxymethyl)piperidine (520 mg, 867 μmol, 95.1%) as a yellow oil. LC-MS (ESI⁺) m/z: 600.2 (M+H)⁺.

Step 2: A mixture of 1-(4-(6′-(benzyloxy)-3′,4′,5,6,7,8-hexahydro-[1,2′-binaphthalen]-1′-yl)phenyl)-4-(dimethoxymethyl)piperidine (500 mg, 1 Eq, 834 μmol), Pd/C (500 mg, 10% Wt, 0.564 Eq, 470 μmol), H₂ (1.68 mg, 1 Eq, 834 μmol) in THE (3 Ml) and MeOH (3 Ml). The suspension was degassed and purged with H₂ for 3 times. The mixture was stirred under H₂ (15 psi) at 50° C. for 24 hour. The reaction was filtered and concentrated to dryness in vacuo to give a white solid. The white solid was subjected by SFC: Column: DAICEL CHIRALCEL OD (250 mm*30 mm, 10 um); Mobile phase: A: Supercritical CO₂, B: MeOH (0.1% NH₃H₂O), A:B=45:45 at 100 Ml/min. The aqueous phase was lyophilized to dryness to give (1R,2S)-1′-(4-(4-(dimethoxymethyl)piperidin-1-yl)phenyl)-1′,2′,3′,4′,5,6,7,8-octahydro-[1,2′-binaphthalen]-6′-ol (150 mg, 293 μmol, 35.2%, 100% Purity) and (1S,2R)-1′-(4-(4-(dimethoxymethyl)piperidin-1-yl)phenyl)-1′,2′,3′,4′,5,6,7,8-octahydro-[1,2′-binaphthalen]-6′-ol (150 mg, 0.29 mmol, 35%, 99% Purity) as a yellow solid. LC-MS (ESI⁺) m/z: 512.2 (M+H)⁺.

Step 3: A mixture of (1R,2S)-1′-(4-(4-(dimethoxymethyl)piperidin-1-yl)phenyl)-1′, 2′,3′,4′,5,6,7,8-octahydro-[1,2′-binaphthalen]-6′-ol (150 mg, 1 Eq, 293 μmol) in 10% H₂SO₄ (1.5 mL) and THE (1.5 mL), then the mixture was stirred at 60° C. for 16 hour. The solvent was removed under vacuum. The residue was dissolved in water (20 mL), the aqueous solution was added NaHCO₃ to adjust pH=6. and washed by ethyl acetate (20 mL*2). The combined organic layer was dried over Na₂SO₄, filtered and concentrated under vacuum to give 1-(4-((1R,2S)-6′-hydroxy-1′,2′,3′,4′,5,6,7,8-octahydro-[1,2′-binaphthalen]-1′-yl)phenyl)piperidine-4-carbaldehyde (100 mg, 215 μmol, 73.3%) as a yellow oil. LC-MS (ESI+) m/z: 466.1 (M+H)+

Step 4: A mixture of 1-(4-((1R,2S)-6′-hydroxy-1′,2′,3′,4′,5,6,7,8-octahydro-[1,2′-binaphthalen]-1′-yl)phenyl)piperidine-4-carbaldehyde (100 mg, 1 Eq, 215 μmol), (R)-3-(1-oxo-5-(piperazin-1-yl)isoindolin-2-yl)piperidine-2,6-dione, TsOH salt (140 mg, 1.3 Eq, 279 μmol) and Sodium acetate (88.1 mg, 5 Eq, 1.07 mmol) and Acetic acid (38.7 mg, 37.1 uL, 3 Eq, 644 μmol) in DCM (2 mL) and MeOH (2 mL) at 25° C. for 1 hour, then Sodium triacetoxyborohydride (91.0 mg, 2 Eq, 430 μmol) was added to the mixture and was stirred at 25° C. for 16 hour. The reaction was treated with H₂O (20 mL), extracted with EtOAc (20 mL*2). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 31%-51%, 7 min). The aqueous phase was lyophilized to dryness to give (R)-3-(5-(4-((1-(4-((1R,2S)-6′-hydroxy-1′,2′,3′,4′,5,6,7,8-octahydro-[1,2′-binaphthalen]-1′-yl)phenyl)piperidin-4-yl)methyl)piperazin-1-yl)-1-oxoisoindolin-2-yl)piperidine-2,6-dione (80.4 mg, 101 μmol, 47.3%, 98.196% Purity) as a white solid.

LC-MS (ESI+) m/z: 778.2 (M+H)⁺

HPLC: 96.196%, purity at 220 nm.

SFC: retention time, 3.939 min; Area, 90.044%; Stereochemistry was arbitrarily assigned ¹H NMR (400 MHZ, DMSO-d₆)₂=10.96 (s, 1H), 9.15-9.08 (m, 1H), 8.16-8.12 (m, 0.255H), 7.55-7.49 (m, 1H), 7.10-7.03 (m, 2H), 6.85-6.80 (m, 1H), 6.73-6.62 (m, 2H), 6.62-6.58 (m, 1H), 6.57-6.50 (m, 2H), 6.50-6.45 (m, 1H), 6.19-6.12 (m, 2H), 6.00-5.93 (m, 1H), 5.09-5.02 (m, 1H), 4.37-4.29 (m, 1H), 4.25-4.17 (m, 1H), 4.16-4.11 (m, 1H), 3.55-3.48 (m, 2H), 3.41-3.32 (m, 11H), 2.98-2.83 (m, 5H), 2.81-2.69 (m, 2H), 2.56 (br s, 1H), 2.38 (br d, J=4.6 Hz, 1H), 2.25-2.17 (m, 2H), 2.17-2.07 (m, 1H), 2.01-1.91 (m, 1H), 1.90-1.81 (m, 1H), 1.81-1.70 (m, 5H), 1.69-1.59 (m, 1H), 1.59-1.49 (m, 1H), 1.23-1.10 (m, 2H).

### Example 233. Preparation of (I-57) (S)-3-(5-(4-((1-(4-((1R,2S)-2-(bicyclo[4.2.0]octa-1 (6), 2,4-trien-3-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

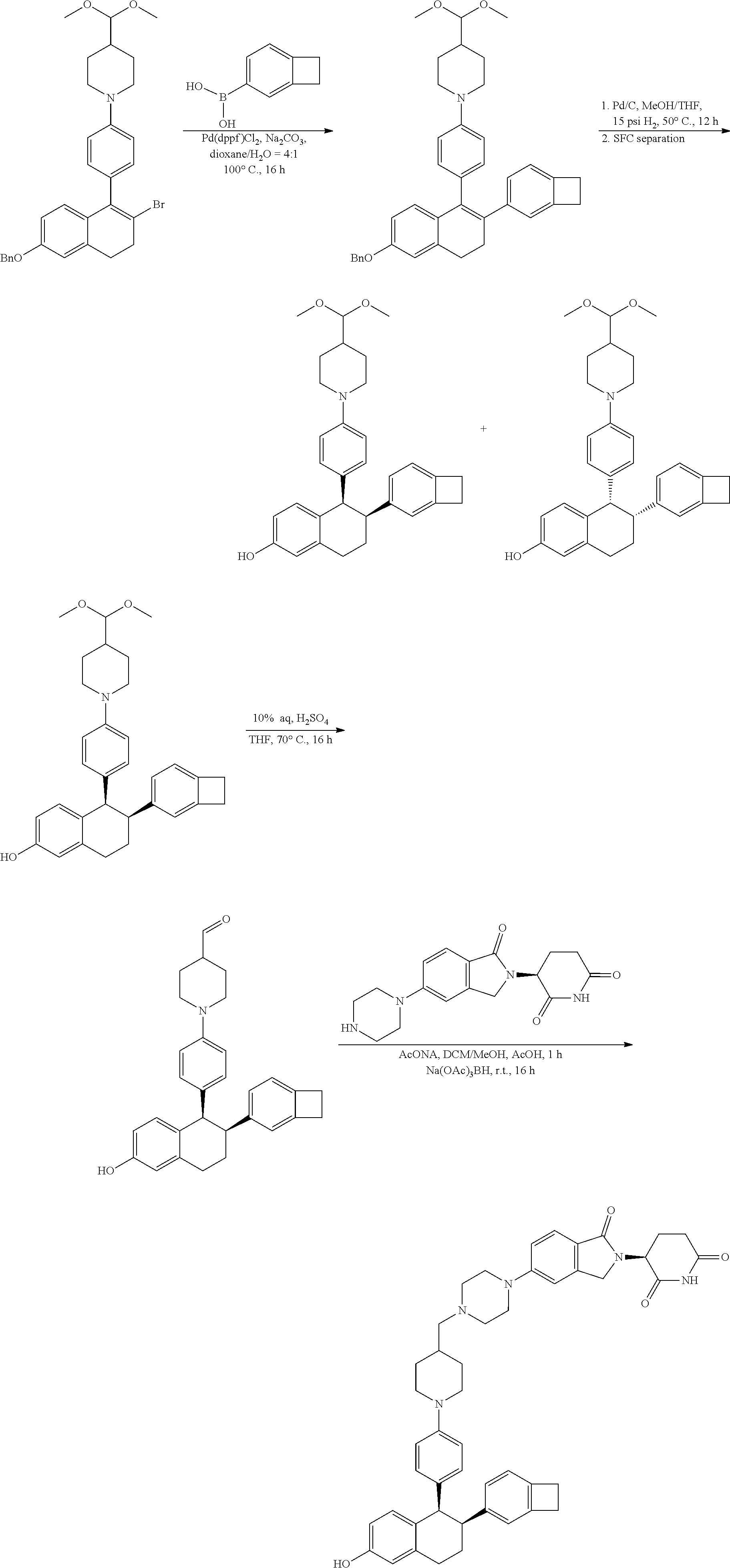

Step 1: A mixture of Bicyclo[4.2.0]octa-1,3,5-trien-3-ylboronic acid (135 mg, 1 Eq, 912 μmol), 1-(4-(6-(benzyloxy)-2-bromo-3,4-dihydronaphthalen-1-yl)phenyl)-4-(dimethoxymethyl)piperidine (500 mg, 1 Eq, 912 μmol), 1,1′-Bis(diphenylphosphino)ferrocene-palladium (II) dichloride (66.7 mg, 0.1 Eq, 91.2 μmol), sodium carbonate (290 mg, 3 Eq, 2.73 mmol) in 1,4-Dioxane (10 mL) and H₂O (2.5 mL) was degassed and purged with N₂ for 3 times, and then the mixture was stirred at 100° C. for 16 hour under N₂ atmosphere. TLC (petroleum ether/ethyl acetate=5/1, R_{f}=0.4) The reaction mixture was quenched by addition H₂O 50 mL, and then diluted with H₂O 50 mL and extracted with Ethyl acetate 50 mL (50 mL*2). The combined organic layers were washed with brine 50 mL, dried over Na₂SO₄, filtered and concentrated under reduced pressure to give a residue. The residue was purified by flash silica gel chromatography (ISCOR; 20 g SepaFlash® Silica Flash Column, Eluent of 50˜50% Ethylacetate/Petroleum ether gradient @ 20 mL/min) and the organic layer was concentrated in vacuo to give 1-(4-(6-(benzyloxy)-2-(bicyclo[4.2.0]octa-1,3,5-trien-3-yl)-3,4-dihydronaphthalen-1-yl)phenyl)-4-(dimethoxymethyl)piperidine (500 mg, 856 μmol, 93.9%, 97.911% purity) as a yellow solid. LC-MS (ESI⁺) m/z: 572.1 (M+H)⁺.

Step 2: A mixture of 1-(4-(6-(benzyloxy)-2-(bicyclo[4.2.0]octa-1 (6), 2,4-trien-3-yl)-3,4-dihydronaphthalen-1-yl)phenyl)-4-(dimethoxymethyl)piperidine (500 mg, 1 Eq, 874 μmol), Pd/C (500 mg, 10% Wt, 0.537 Eq, 470 μmol) in MeOH (10 mL) and THF (10 mL) was degassed and purged with H₂ (15 psi) for 3 times, and then the mixture was stirred at 50° C. for 12 hr under H2 atmosphere. The reaction mixture was filtered and concentrated to dryness in vacuo to give a white solid. The white solid was subjected by SFC: Column: DAICEL CHIRALCEL OD (250 mm*30 mm, 10 um); Mobile phase: A: Supercritical CO₂, B: EtOH (0.1% NH₃H₂O), A:B=65:35 at 100 mL/min. The aqueous phase was lyophilized to dryness to give (5R,6S)-6-(bicyclo[4.2.0]octa-1 (6), 2,4-trien-3-yl)-5-(4-(4-(dimethoxymethyl)piperidin-1-yl)phenyl)-5,6,7,8-tetrahydronaphthalen-2-ol (110 mg, 227 μmol, 26.0%) and (5S,6R)-6-(bicyclo[4.2.0]octa-1,3,5-trien-3-yl)-5-(4-(4-(dimethoxymethyl)piperidin-1-yl)phenyl)-5,6,7,8-tetrahydronaphthalen-2-ol (98 mg, 0.20 mmol, 23%) was obtained as a yellow oil.

LC-MS (ESI⁺) m/z: 484.3 (M+H)⁺

Step 3: A solution (5R,6S)-6-(bicyclo[4.2.0]octa-1,3,5-trien-3-yl)-5-(4-(4-(dimethoxymethyl)piperidin-1-yl)phenyl)-5,6,7,8-tetrahydronaphthalen-2-ol (110 mg, 1 Eq, 227 μmol) in 10% H₂SO₄ (7 mL) and THF (7 mL) was stirred at 70° C. for 16 hour to give a yellow suspension. The solvent was removed under vacuum. The aqueous solution was added NaHCO₃ to adjust pH=6. The residue was dissolved in water (20 mL) and extracted by ethyl acetate (50 mL). The combined organic layer was dried over Na₂SO₄, filtered and concentrated under vacuum to give 1-(4-((1R,2S)-2-(bicyclo[4.2.0]octa-1,3,5-trien-3-yl)-6-hydroxy-1,2,3,4-tetrahydronaphthalen-1-yl)phenyl)piperidine-4-carbaldehyde (100 mg, 224 μmol, 98.7%, 98.227% Purity) as a yellow oil. LC-MS (ESI⁺) m/z: 438.1 (M+H)⁺

Step 4: To a solution of 1-(4-((1R,2S)-2-(bicyclo[4.2.0]octa-1,3,5-trien-3-yl)-6-hydroxy-1,2,3,4-tetrahydronaphthalen-1-yl)phenyl)piperidine-4-carbaldehyde (100 mg, 1 Eq, 229 μmol), (S)-3-(1-oxo-5-(piperazin-1-yl)isoindolin-2-yl)piperidine-2,6-dione, TsOH salt (114 mg, 1 Eq, 229 μmol) in DCM (3 mL) and MeOH (3 mL) was added Sodium acetate (56.2 mg, 3 Eq, 686 μmol) was stirred at 25° C. for 60 mins, Then Sodium triacetoxyborohydride (96.9 mg, 2 Eq, 457 μmol) and Acetic acid (41.2 mg, 39.4 uL, 3 Eq, 686 μmol) was added. The mixture was stirred at 25° C. for 16 hour. The reaction was treated with H₂O (20 mL), extracted with EtOAc (30 mL). The combined extracts was dried over anhydrous Na₂SO₄, filtered and concentrated to dryness in vacuo to give a residue. The reaction mixture was concentrated under reduced pressure to remove solvent. The residue was purified by prep-HPLC (column: Welch Xtimate C18 150*30 mm*5 um; mobile phase: [water(FA)-ACN]; B % 26%-56%, 7 min) to give (S)-3-(5-(4-((1-(4-((1R,2S)-2-(bicyclo[4.2.0]octa-1 (6), 2,4-trien-3-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (48.9 mg, 65.0 μmol, 28.4%, 99.660% Purity) was obtained as a white solid. LC-MS (ESI⁺) m/z: 750.2 (M+H)⁺.

LCMS: calc. for C₄₇H₅₁N₅O₄:749.94, found: [M+H]⁺ 750.2.

HPLC: 99.660% purity at 220 nm.

¹H NMR (400 MHZ, DMSO-d₆) δ=10.98-10.92 (m, 1H), 8.23-8.17 (m, 0.377H), 7.56-7.48 (m, 1H), 7.11-7.01 (m, 2H), 6.90-6.80 (m, 1H), 6.69-6.62 (m, 2H), 6.61-6.53 (m, 3H), 6.51-6.43 (m, 2H), 6.28-6.19 (m, 2H), 5.10-5.00 (m, 1H), 4.38-4.28 (m, 1H), 4.26-4.15 (m, 1H), 4.11-4.05 (m, 1H), 3.59-3.49 (m, 3H), 3.47-3.36 (m, 6H), 3.10-2.81 (m, 8H), 2.70-2.58 (m, 2H), 2.47-2.27 (m, 4H), 2.24-2.16 (m, 2H), 2.11-1.91 (m, 2H), 1.81-1.72 (m, 2H), 1.70-1.59 (m, 2H), 1.26-1.10 (m, 2H)

SFC: retention time, 3.665 min; Area, 94.697%; Stereochemistry was arbitrarily assigned

### Example 234. Preparation of (I-100) (R)-3-(5-(4-((1-(4-((1R,2S)-2-(bicyclo[4.2.0]octa-1,3,5-trien-2-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

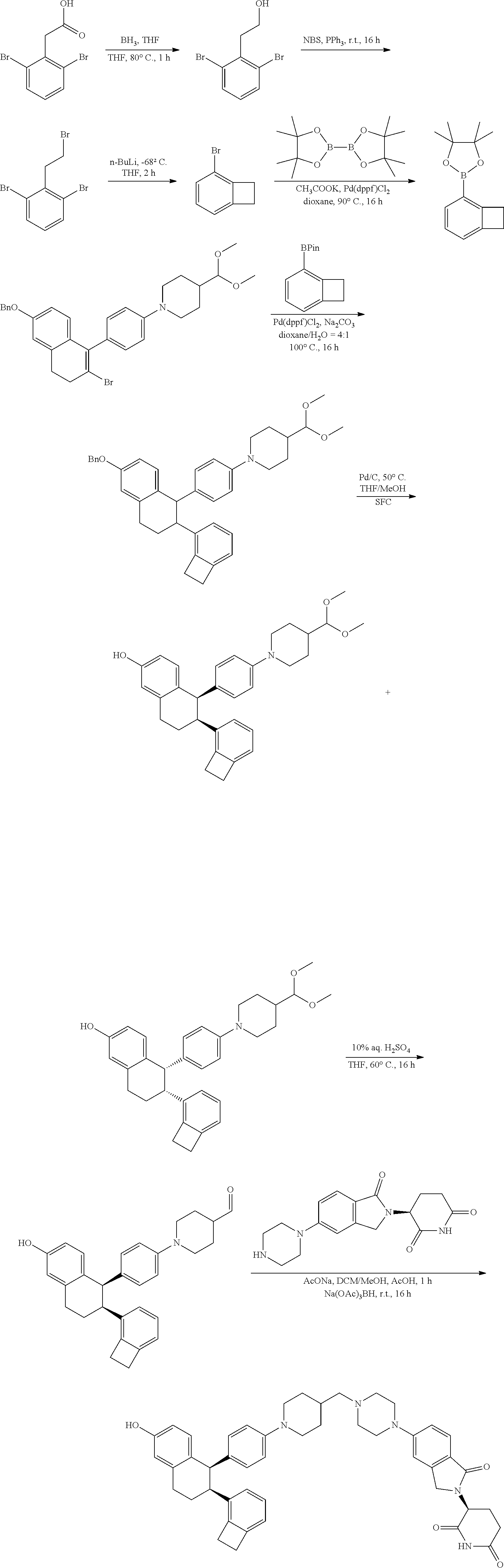

Step 1: To solution of 2-(2,6-dibromophenyl)acetic acid (3 g, 1 Eq, 0.01 mol) in THF (30 mL) was added dropwise over 30 min. BH₃·THF (1.3 g, 15 mL, 1 Eq, 15 mmol) at 0° C., after 10 min, the reaction mixture was warmed to 80° C., refluxed at 80° C. for 1 hour. The mixture was treated with H₂O (200 mL), extracted with EtOAc (150 mL). The organic lay was dried over anhydrous Na₂SO₄, filtered and concentrated to dryness in vacuo to give 2-(2,6-dibromophenyl)ethan-1-ol (2.4 g, 8.6 mmol, 80%) as a yellow solid. LC-MS (ESI+) m/z: 262.9 (M+H)⁺.

Step 2: To solution of 2-(2,6-dibromophenyl)ethan-1-ol (2.4 g, 1 Eq, 8.6 mmol), N-Bromosuccinimide (1.7 g, 0.80 mL, 1.1 Eq. 9.4 mmol) in DCM (20 mL) was added dropwise over 10 min. Triphenylphosphine (2.5 g, 2.1 mL, 1.1 Eq, 9.4 mmol) at 0° C. The reaction mixture was stirred at 25° C. for 16 hour. TLC (PE/EtOAc=20/1) showed a new spot. The mixture was treated with H₂O (100 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜5% ethyl acetate in petroleum ether). The pure fractions were collected and concentrated to dryness in vacuo to give 1,3-dibromo-2-(2-bromoethyl)benzene (2.2 g, 6.4 mmol, 75%) as a yellow oil.

¹H NMR (400 MHZ, CHLOROFORM-d₃) δ=7.50-7.40 (m, 2H), 6.96-6.85 (m, 1H), 3.54-3.38 (m, 4H)

Step 3: 1,3-dibromo-2-(2-bromoethyl)benzene (1 g, 1 Eq, 3 mmol), a stir bar and THE (10 mL) were add to a 50 mL three necked bottle. The resultant mixture was sparged with N₂ for three times and was subsequently cooled to −68° C., n-butyllithium (0.18 g, 1.1 mL, 2.5 molar, 0.9 Eq, 2.8 mmol) was added to the reaction over 10 min. The resulting mixture was stirred for 2 hour at −68° C. The mixture was treated with Saturated aqueous ammonium chloride (10 mL), then was added H₂O (100 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜10% ethyl acetate in petroleum ether). The pure fractions were collected and concentrated to dryness in vacuo to give 2-bromobicyclo[4.2.0]octa-1,3,5-triene (400 mg, 2.19 mmol, 70%) was obtained as yellow oil.

Step 4: To a solution of 2-bromobicyclo[4.2.0]octa-1,3,5-triene (700 mg, 1 Eq, 3.82 mmol), Bis(pinacolato) diborane (1.46 g, 1.5 Eq, 5.74 mmol), Potassium acetate (751 mg, 478 μL, 2 Eq, 7.65 mmol) in Dioxane (5 mL). [1,1′-bis(diphenylphosphino)ferrocene]dichloropalladium(II) Complex With Dichloromethane (312 mg, 0.1 Eq, 382 μmol) was added to the reaction. Then the resultant mixture was stirred for 16 hour at 90° C. under N₂. The reaction mixture was quenched by addition of water (100 mL), extracted with EtOAc (100 mL). The combined organic layers were washed with brine (50 mL), dried over Na₂SO₄, filtered and concentrated under reduced pressure to give a residue. The residue was purified by flash silica gel chromatography (from PE/EtOAc=20/1 to 10/1) to yield a product. 2-(bicyclo[4.2.0]octa-1,3,5-trien-2-yl)-4,4,5,5-tetramethyl-1,3,2-dioxaborolane (500 mg, 1.7 mmol, 45% yield, 80% purity) was obtained as yellow oil. LC-MS (ESI+) m/z: 231.1 (M+H)+.

Step 5: A mixture of 1-(4-(6-(benzyloxy)-2-bromo-3,4-dihydronaphthalen-1-yl)phenyl)-4-(dimethoxymethyl)piperidine (800 mg, 1 Eq, 1.46 mmol), 2-(bicyclo[4.2.0]octa-1,3,5-trien-2-yl)-4,4,5,5-tetramethyl-1,3,2-dioxaborolane (500 mg 1.19 Eq, 1.74 mmol), Na₂CO₃ (464 mg, 3 Eq, 4.38 mmol) and [1,1′-bis(diphenylphosphino)ferrocene]dichloropalladium(II)ComplexWithDichloromethane (119 mg, 0.1 Eq, 146 μmol) in Dioxane (4 mL) and H₂O (1 mL) at r.t. N₂ was bubbled into the mixture for 5 min. The reaction mixture was heated at 100° C. for 16 hour. Then the reaction was cooled to room temperature. The mixture was treated with H₂O (100 mL), extracted with EtOAc (100 mL). The combined extracts was dried over anhydrous Na₂SO₄, filtered and concentrated to dryness in vacuo to give a residue. The residue was subjected to column chromatography over silica gel (gradient elution: 0˜20% ethyl acetate in petroleum ether). The pure fractions were collected and concentrated to dryness in vacuo to give 1-(4-(6-(benzyloxy)-2-(bicyclo[4.2.0]octa-1,3,5-trien-2-yl)-3,4-dihydronaphthalen-1-yl)phenyl)-4-(dimethoxymethyl)piperidine (400 mg, 0.52 mmol, 36% yield, 75% purity) as a yellow solid. LC-MS (ESI⁺) m/z: 572.2 (M+H)⁺.

Step 6: A mixture of 1-(4-(6-(benzyloxy)-2-(bicyclo[4.2.0]octa-1 (6), 2,4-trien-2-yl)-3,4-dihydronaphthalen-1-yl)phenyl)-4-(dimethoxymethyl)piperidine (400 mg, 1 Eq, 525 μmol), Pd/C (300 mg, 10% Wt, 0.537 Eq, 282 μmol), H2 (1.06 mg, 1 Eq, 525 μmol) in THF (2 mL) and MeOH (2 mL). The suspension was degassed and purged with H₂ for 3 times. The mixture was stirred under H₂ (15 psi) at 50° C. for 12 hour. Then the reaction was cooled to room temperature. The reaction was filtered and concentrated to dryness in vacuo to give a white solid. The white solid was subjected by SFC: Column: DAICEL CHIRALCEL OD (250 mm*30 mm, 10 um); Mobile phase: A: Supercritical CO₂, B: MeOH (0.1% NH₃H₂O), A:B=35:35 at 80 mL/min. The aqueous phase was lyophilized to dryness to give (5S,6R)-6-(bicyclo[4.2.0]octa-1 (6), 2,4-trien-2-yl)-5-(4-(4-(dimethoxymethyl)piperidin-1-yl)phenyl)-5,6,7,8-tetrahydronaphthalen-2-ol (55 mg, 0.11 mmol, 21% yield, 98% purity) and (5R,6S)-6-(bicyclo[4.2.0]octa-1 (6), 2,4-trien-2-yl)-5-(4-(4-(dimethoxymethyl)piperidin-1-yl)phenyl)-5,6,7,8-tetrahydronaphthalen-2-ol (55 mg, 0.11 mmol, 21% yield, 99% purity) as a yellow solid. LC-MS (ESI⁺) m/z: 484.2 (M+H)⁺.

Step 7: A mixture of (5R,6S)-6-(bicyclo[4.2.0]octa-1,3,5-trien-2-yl)-5-(4-(4-(dimethoxymethyl)piperidin-1-yl)phenyl)-5,6,7,8-tetrahydronaphthalen-2-ol (55 mg, 1 Eq, 0.11 mmol) in 10% H₂SO₄ (1.5 mL) and THF (1.5 mL), then the mixture was stirred at 60° C. for 16 hour. The solvent was removed under vacuum. The residue was dissolved in water (20 mL), the aqueous solution was added NaHCO₃ to adjust pH=6, and extracted by ethyl acetate (20 mL*2). The combined organic layer was dried over Na₂SO₄, filtered and concentrated under vacuum to give 1-(4-((1R,2S)-2-(bicyclo[4.2.0]octa-1,3,5-trien-2-yl)-6-hydroxy-1,2,3,4-tetrahydronaphthalen-1-yl)phenyl)piperidine-4-carbaldehyde (50 mg, 0.11 mmol, 100%) as a yellow oil. LC-MS (ESI+) m/z: 438.1 (M+H)+.

Step 8: A mixture of 1-(4-((1R,2S)-2-(bicyclo[4.2.0]octa-1,3,5-trien-2-yl)-6-hydroxy-1,2,3,4-tetrahydronaphthalen-1-yl)phenyl)piperidine-4-carbaldehyde (50 mg, 1 Eq, 0.11 mmol), (R)-3-(1-oxo-5-(piperazin-1-yl)isoindolin-2-yl)piperidine-2,6-dione, TsOH salt (74 mg, 1.3 Eq, 0.15 mmol) and Sodium acetate (47 mg, 5 Eq, 0.57 mmol) and Acetic acid (21 mg, 20 μL, 3 Eq, 0.34 mmol) in DCM (2 mL) and MeOH (2 mL) at 25° C. for 1 hour, then Sodium triacetoxyborohydride (48 mg, 2 Eq, 0.23 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 26%-46%, 7 min). The aqueous phase was lyophilized to dryness to give (R)-3-(5-(4-((1-(4-((1R,2S)-2-(bicyclo[4.2.0]octa-1,3,5-trien-2-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (28.1 mg, 36.8 μmol, 32%, 98.336% Purity) as a white solid.

LC-MS (ESI+) m/z: 750.2 (M+H)+

HPLC: 98.336%, purity at 220 nm.

SFC: retention time, 3.096 min; Area, 88.165%; Stereochemistry was arbitrarily assigned.

¹H NMR (400 MHZ, DMSO-d₆) δ=10.99-10.92 (m, 1H), 9.16-9.08 (m, 1H), 8.15-8.13 (m, 0.260H), 7.59-7.49 (m, 1H), 7.14-7.02 (m, 3H), 6.95-6.89 (m, 1H), 6.86-6.80 (m, 1H), 6.70-6.64 (m, 1H), 6.62-6.55 (m, 3H), 6.53-6.46 (m, 1H), 6.22-6.14 (m, 2H), 5.12-5.00 (m, 1H), 4.38-4.29 (m, 1H), 4.27-4.17 (m, 1H), 4.08-4.01 (m, 1H), 3.67-3.42 (m, 4H), 3.28-3.14 (m, 3H), 3.05-2.80 (m, 5H), 2.79-2.64 (m, 4H), 2.63-2.54 (m, 4H), 2.45-2.22 (m, 3H), 2.11-1.92 (m, 2H), 1.84-1.65 (m, 5H), 1.30-1.12 (m, 2H).

### Example 235. Preparation of (I-161) (S)-3-(5-(4-((1-(4-((1R,2S)-2-((3R,5R,7R)-adamantan-1-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

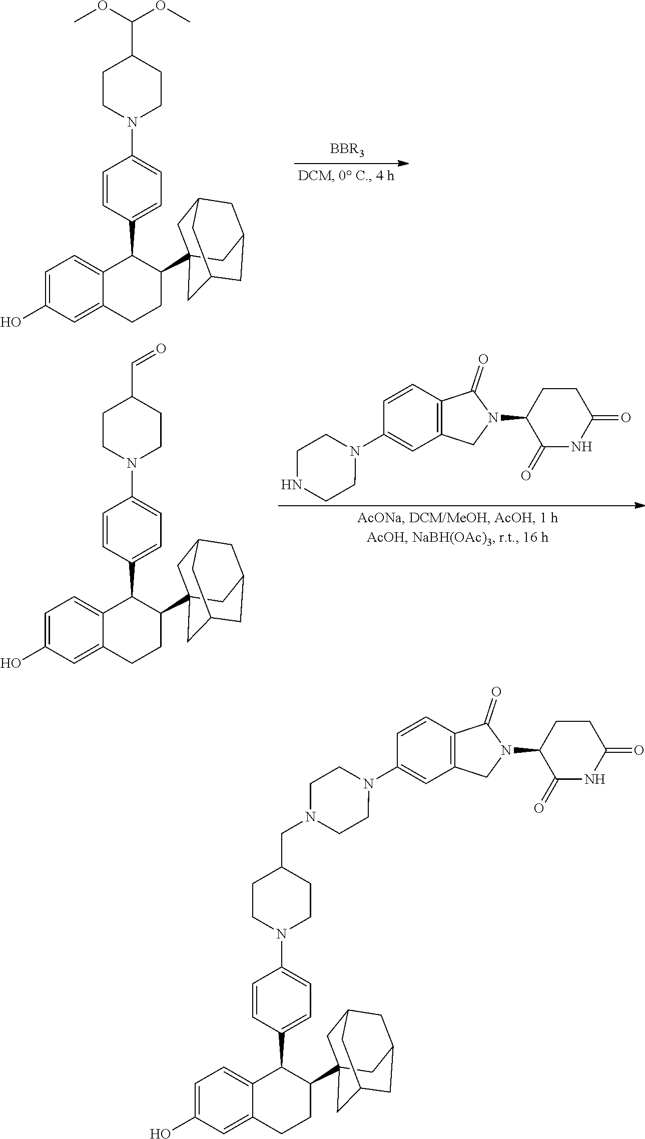

Step 1: A mixture of 1-(4-((1R,2S)-2-((3R,5R,7R)-adamantan-1-yl)-6-methoxy-1,2,3,4-tetrahydronaphthalen-1-yl)phenyl)-4-(dimethoxymethyl)piperidine (30 mg, 1 Eq, 57 μmol) in DCM (2 mL) at 0° C. was added tribromoborane (0.17 mL, 1 M, 3 Eq, 0.17 mmol) with N₂. After addition, the mixture was stirred at 0° C. for 4 hour, and then the reaction was warmed to 25° C. and was stirred at this temperature for 4 hour. The reaction was treated with aq. NaHCO₃ (10 mL), extracted with EtOAc (20 mL*2). The combined extracts was dried over anhydrous Na₂SO₄, filtered and concentrated to dryness in vacuo to give 1-(4-((1R,2S)-2-((3R,5R,7R)-adamantan-1-yl)-6-hydroxy-1,2,3,4-tetrahydronaphthalen-1-yl)phenyl)piperidine-4-carbaldehyde (25 mg, 53 μmol, 94% yield) as yellow oil.

Step 2: A mixture of 1-(4-((1R,2S)-2-((3R,5R,7R)-adamantan-1-yl)-6-hydroxy-1,2,3,4-tetrahydronaphthalen-1-yl)phenyl)piperidine-4-carbaldehyde (22 mg, 1 Eq, 47 μmol), (S)-3-(1-oxo-5-(piperazin-1-yl)isoindolin-2-yl)piperidine-2,6-dione, PhSO₃H salt (23 mg, 1 Eq, 47 μmol) in DCM (1.5 mL), MeOH (1.5 mL) was added sodium acetate (12 mg, 3 Eq, 0.14 mmol) was stirred at 25° C. for 30 mins, Then sodium triacetoxyborohydride (20 mg, 2 Eq, 94 μmol) and acetic acid (8.4 mg, 8.1 uL, 3 Eq, 0.14 mmol) was added. The mixture was stirred at 25° C. for 16 hour. TLC (DCM/MeOH=10/1, R_{f}=0.4) showed new spot was formed. The reaction mixture was concentrated under reduced pressure to remove solvent. The residue was purified by prep-HPLC (column: Welch Xtimate C18 150*30 mm*5 um; mobile phase: [water(FA)-ACN]; B % 18%-48%, 7 min) to give (S)-3-(5-(4-((1-(4-((1R,2S)-2-((3R,5R,7R)-adamantan-1-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (13.2 mg, 16.9 μmol, 36% yield) was obtained as a white solid.

LC-MS (ESI+) m/z: 782.3 (M+H)⁺

HPLC: 99.429%, purity at 220 nm.

SFC: retention time, 3.564 min; Area, 96.797%;

¹H NMR (400 MHz, DMSO-d₆) δ=10.96 (s, 1H), 8.98 (br s, 1H), 8.22 (s, 1H), 7.52 (d, J=8.6 Hz, 1H), 7.14-6.90 (m, 4H), 6.76 (br d, J=8.5 Hz, 2H), 6.58 (d, J=8.5 Hz, 1H), 6.48 (d, J=1.9 Hz, 1H), 6.37 (dd, J=2.2, 8.2 Hz, 1H), 5.05 (dd, J=5.0, 13.2 Hz, 1H), 4.43-4.28 (m, 1H), 4.25-4.15 (m, 1H), 4.11 (br d, J=3.8 Hz, 1H), 3.61 (brt, J=13.2 Hz, 2H), 3.29 (br s, 6H), 3.01-2.83 (m, 3H), 2.81-2.64 (m, 2H), 2.58 (br d, J=17.0 Hz, 4H), 2.37 (br dd, J=4.9, 12.8 Hz, 1H), 2.20 (br d, J=6.8 Hz, 2H), 1.99-1.87 (m, 2H), 1.85-1.76 (m, 6H), 1.63-1.45 (m, 10H), 1.35 (br d, J=11.8 Hz, 3H), 1.22 (br d, J=12.9 Hz, 2H)

### Example 236. Preparation of (I-241) (S)-3-(5-(4-((1-(4-((1S,2R)-2-((3S,5S,7S)-adamantan-1-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

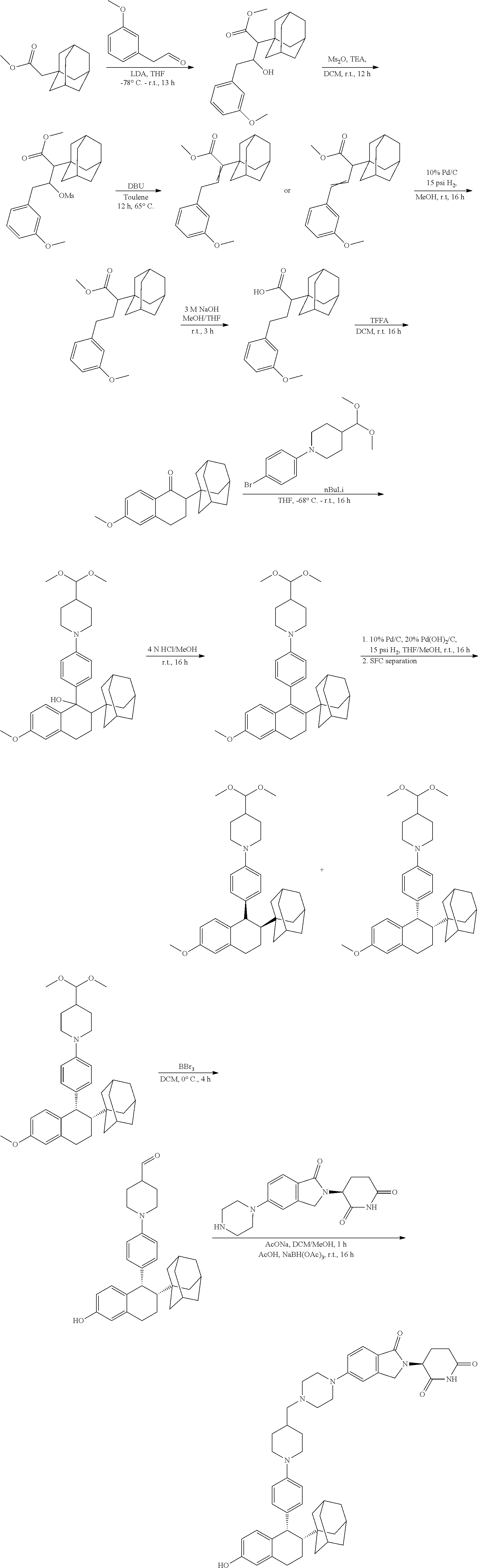

Step 1: To a mixture of methyl 2-((3r, 5r, 7r)-adamantan-1-yl)acetate (10 g, 1 Eq, 48 mmol) in THF (100 mL) at r.t., and the mixture was degassed and purged with N₂ for 10 minutes. Then the mixture was stirred until it reached −78° C. Lithium diisopropylamide solution (6.7 g, 31.2 mL, 2 M, 1.3 Eq, 62.4 mmol) was added to the mixture and stirred at −78° C. for 1 hour, then 2-(3-methoxyphenyl)acetaldehyde (7.2 g, 1 Eq, 48 mmol) was added to the mixture at −78° C. and stirred at 25° C. for 12 hour. TLC (petroleum ether:ethyl acetate=10:1, R_{f}=0.5 UV) showed one main new spot was observed. The reaction was quenched with aqueous NH₄Cl (50 mL) and extracted with ethyl acetate (150 mL*2). The organic layer was washed with brine (100 mL) and dried over Na₂SO₄ and concentrated in vacuo to give a black solid. The black solid was subjected to column chromatography over silica gel (gradient elution: 0-20% EtOAc). The desired fractions were collected, and concentrated to dryness in vacuo to give methyl 2-((3r, 5r, 7r)-adamantan-1-yl)-3-hydroxy-4-(3-methoxyphenyl)butanoate (11 g, 31 mmol, 64% yield) as a yellow oil.

Step 2: A mixture of methyl 2-((3r, 5r, 7r)-adamantan-1-yl)-3-hydroxy-4-(3-methoxyphenyl) butanoate (2.6 g, 1 Eq, 7.2 mmol), methanesulfonic anhydride (2.5 g, 2 Eq, 14.4 mmol), triethylamine (2.2 g, 3.0 mL, 3 Eq, 21.6 mmol) and a stir bar were added into DCM (50 mL). Then the mixture was stirred at 25° C. for 2 hour. TLC (petroleum ether:ethyl acetate=10:1, R_{f}=0.4 UV) showed one main new spot was observed. To the mixture was added water (50 mL), then was extracted with DCM (50 mL*2). The combined organic layers were washed with brine (50 mL), dried over Na₂SO₄, filtered and concentrated under reduced pressure to give the crude product. The residue was purified by flash silica gel chromatography (petroleum ether/ethyl acetate from 100/0 to 90/10) to obtained the desired product methyl 2-((3r, 5r, 7r)-adamantan-1-yl)-4-(3-methoxyphenyl)-3-((methylsulfonyl)oxy)butanoate (2.6 g, 6.0 mmol, 83% yield) as brown oil.

Step 3: A mixture of methyl 2-((3r, 5r, 7r)-adamantan-1-yl)-4-(3-methoxyphenyl)-3-(tosyloxy) butanoate (2.5 g, 1 Eq, 4.9 mmol), DBU (2.2 g, 2.2 mL, 3 Eq, 15 mmol) in Toulene (20 mL). The suspension was degassed and purged with N₂ for 3 times. The mixture was stirred under N₂ at 25° C. for 12 hour. TLC (PE/EtOAc=10/1, R_{f}=0.4) showed a new spot was formed. To the mixture was added water (100 mL), then was extracted with ethyl acetate (50 mL*2). The combined organic layers were washed with brine (50 mL), dried over Na₂SO₄, filtered and concentrated under reduced pressure to give the crude product. The desired product was purified by flash column (petroleum ether/ethyl acetate from 100/0 to 90/10) and the organic layer was concentrated in vacuo to give methyl 2-((3r, 5r, 7r)-adamantan-1-yl)-4-(3-methoxyphenyl)but-3-enoate (1.1 g, 3.2 mmol, 66% yield) as a yellow oil.

Step 4: A mixture of methyl 2-((3r, 5r, 7r)-adamantan-1-yl)-4-(3-methoxyphenyl)but-3-enoate (1 g, 1 Eq, 2.9 mmol) in MeOH (10 mL) was added Pd/C (1.6 g, 10% Wt, 0.5 Eq, 1.5 mmol) under N₂ atmosphere. The suspension was degassed and purged with H₂ for 10 mins. The mixture was stirred under 15 psi H2 (5.9 mg, 1 Eq, 2.9 mmol) at 50° C. for 16 hr. TLC (petroleum ether:ethyl acetate=5:1, R_{f}=0.4 UV) showed one main new spot was observed. The reaction was filtrate and concentrated to obtained methyl 2-((3r, 5r, 7r)-adamantan-1-yl)-4-(3-methoxyphenyl)butanoate (600 mg, 1.75 mmol, 59.6% yield) as yellow oil.

Step 5: A mixture of methyl 2-((3r, 5r, 7r)-adamantan-1-yl)-4-(3-methoxyphenyl)butanoate (600 mg, 1 Eq, 1.75 mmol) in DMSO (6 mL) was added sodium ethane thiolate (295 mg, 2 Eq, 3.5 mmol). The mixture was stirred at 100° C. for 16 hour. TLC (petroleum ether/ethyl acetate=3:1, R_{f}=0.3) showed new spot was formed. The mixture was adjusted to pH=˜4 with 2N HCl. 50 mL water was added, and the mixture was extracted with ethyl acetate (2×50 mL). The combined organic layers were washed with brine (50 mL), dried over anhydrous Na₂SO₄, filtered and concentrated to dryness in vacuo to give 2-((3r, 5r, 7r)-adamantan-1-yl)-4-(3-methoxyphenyl)butanoic acid (400 mg, 1.22 mmol, 69.5% yield) as yellow oil.

Step 6: A mixture of 2, 2, 2-trifluoroacetic anhydride (959 mg, 635 μL, 5 Eq, 4.57 mmol) was added to 2-((3r, 5r, 7r)-adamantan-1-yl)-4-(3-methoxyphenyl)butanoic acid (300 mg, 1 Eq, 913 μmol) in DCM (10 mL). The reaction was stirred at 0° C. for 30 min. The reaction was stirred at 30° C. for 16 hour. TLC (petroleum ether:ethyl acetate=10:1, UV) showed one main new spot was observed. 50 mL water was added, and the mixture was extracted with ethyl acetate (50 mL×2). The combined organic layers were washed with brine (50 mL), dried over anhydrous Na₂SO₄, filtered and concentrated to dryness in vacuo to give 2-((3r, 5r, 7r)-adamantan-1-yl)-6-methoxy-3,4-dihydronaphthalen-1 (2H)-one (200 mg, 644 μmol, 70.5% yield) as a yellow oil.

Step 7: A mixture of 1-(4-bromophenyl)-4-(dimethoxymethyl)piperidine (395 mg, 3 Eq, 1.26 mmol), a stir bar, and THF (6 mL) were added an oven-dried and nitrogen-purged 20 mL three round-bottomed flask, which was subsequently evacuated and refilled with argon (×3), before the reaction vessel was cooled to −68° C. (dry ice/EtOH). The resulting mixture treated with n-Butyl lithium, 2.5 M in hexane (419 μL, 2.5 M, 2.5 Eq, 1.05 mmol), dropwise over 2 min at −68° C. (dry ice/EtOH). While stirring an additional 30 min at −68° C. (dry ice/EtOH). The resulting mixture treated with 2-((3r, 5r, 7r)-adamantan-1-yl)-6-methoxy-3,4-dihydronaphthalen-1 (2H)-one (130 mg, 1 Eq, 419 μmol) in THF (2 mL), dropwise over 5 min, and the reaction vessel removed to the r.t., while stirring an additional 15 h at r.t. TLC (petroleum ether/ethyl acetate=5/1, R_{f}=0.4). The reaction mixture treated with sat. aq. NH₄Cl (20 mL), dropwise over 3 min, extracted with EtOAc (50 mL×3), and the combined extracts washed with brine (20 mL), dried over anhydrous Na₂SO₄, filtered, and concentrated to dryness in vacuo to give a crude product, which was then subjected to silica gel chromatography (0-50% EtOAc/pet ether) to give 2-((3r, 5r, 7r)-adamantan-1-yl)-1-(4-(4-(dimethoxymethyl)piperidin-1-yl)phenyl)-6-methoxy-1,2,3,4-tetrahydronaphthalen-1-ol (180 mg, 330 μmol, 78.8% yield) as a yellow solid.

Step 8: A mixture of 2-((3r, 5r, 7r)-adamantan-1-yl)-1-(4-(4-(dimethoxymethyl)piperidin-1-yl)phenyl)-6-methoxy-1,2,3,4-tetrahydronaphthalen-1-ol (140 mg, 1 Eq, 257 μmol) in 4 N HCl/MeOH (5 mL) at r.t., and the reaction was stirred at 25° C. for 5 hour. TLC (petroleum ether/ethyl acetate=5/1, R_{f}=0.3). The mixture was filtered and the filter cake was washed with EtOAc (50 mL). The filtrate was treated with H₂O (30 mL), extracted with EtOAc (50 mL*2). The combined extracts was dried over anhydrous Na₂SO₄, filtered and concentrated to dryness in vacuo to give a residue. The residue was subjected to column chromatography over silica gel (petroleum ether/ethyl acetate from 100/0 to 70/30). The pure fractions were collected and concentrated to dryness in vacuo to give 1-(4-(2-((3r, 5r, 7r)-adamantan-1-yl)-6-methoxy-3,4-dihydronaphthalen-1-yl)phenyl)-4-(dimethoxymethyl)piperidine (100 mg, 189 μmol, 73.9% yield) as a yellow solid.

Step 9: A mixture of Pd/C (80 mg, 10% Wt, 0.50 Eq, 75 μmol), Pd(OH)₂/C (106.4 mg, 20% Wt, 1 Eq, 151.6 μmol) and 1-(4-(2-((3r, 5r, 7r)-adamantan-1-yl)-6-methoxy-3,4-dihydronaphthalen-1-yl)phenyl)-4-(dimethoxymethyl)piperidine (80 mg, 1 Eq, 151.6 μmol) in EtOH (2 mL) and EtOAc (2 mL) was degassed and purged with H₂ (15 psi) for 3 times, and then the mixture was stirred at 30° C. for 16 hour under H₂ atmosphere. The reaction mixture was filtered and concentrated under reduced pressure to give a residue. The residue was concentrated under reduced pressure to give desired compound (0.1 g, purity ˜90%) as a yellow oil, which was further separated by SFC (condition: column: DAICEL CHIRALCELOJ (250 mm*30 mm, 10 um); mobile phase: [0.1% NH₃H₂O EtOH]; B %: 35%-35%, min) to give 1-(4-((1S,2R)-2-((3S,5S,7S)-adamantan-1-yl)-6-methoxy-1,2,3,4-tetrahydronaphthalen-1-yl)phenyl)-4-(dimethoxymethyl)piperidine (33 mg, 62 μmol, 41% yield) was obtained as a yellow oil and 1-(4-((1R,2S)-2-((3R,5R,7R)-adamantan-1-yl)-6-methoxy-1,2,3,4-tetrahydronaphthalen-1-yl)phenyl)-4-(dimethoxymethyl)piperidine (30 mg, 57 μmol, 37% yield) was obtained as a yellow oil.

Step 10: A mixture of 1-(4-((1S, 2R)-2-((3R, 5R, 7R)-adamantan-1-yl)-6-methoxy-1, 2, 3, 4-tetrahydronaphthalen-1-yl)phenyl)-4-(dimethoxymethyl)piperidine (30 mg, 1 Eq, 57 μmol) in DCM (2 mL) at 0° C. was added tribromoborane (43 mg, 0.17 mL, 1 molar, 3 Eq, 0.17 mmol) with N₂. After addition, the mixture was stirred at 0° C. for 4 hour TLC (petroleum ether/ethyl acetate=3/1, R_{f}=0.3) showed new spot was formed. The reaction was treated with aq. NaHCO₃ (10 mL), extracted with EtOAc (20 mL*2). The combined extracts was dried over anhydrous Na₂SO₄, filtered and concentrated to dryness in vacuo to give 1-(4-((1S,2R)-2-((3R,5R,7R)-adamantan-1-yl)-6-hydroxy-1,2,3,4-tetrahydronaphthalen-1-yl)phenyl)piperidine-4-carbaldehyde (25 mg, 53 μmol, 94%) as a yellow oil.

Step 11: A mixture of 1-(4-((1S,2R)-2-((3S,5S,7S)-adamantan-1-yl)-6-methoxy-1,2,3,4-tetrahydronaphthalen-1-yl)phenyl)-4-(dimethoxymethyl)piperidine (25 mg, 1 Eq, 47 μmol), (S)-3-(1-oxo-5-(piperazin-1-yl)isoindolin-2-yl)piperidine-2,6-dione, PhSO₃H salt (24 mg, 1 Eq, 47 μmol) in DCM (1.5 mL), MeOH (1.5 mL) was added sodium acetate (12 mg, 3 Eq, 0.14 mmol) was stirred at 25° C. for 30 mins, Then sodium triacetoxyborohydride (20 mg, 2 Eq, 94 μmol) and acetic acid (8.5 mg, 8.1 μL, 3 Eq, 0.14 mmol) was added. The mixture was stirred at 25° C. for 16 hour. TLC (DCM/MeOH=10/1, R (0.4) showed new spot was formed. The reaction mixture was concentrated under reduced pressure to remove solvent. The residue was purified by prep-HPLC (column: Welch Xtimate C18 150*30 mm*5 um; mobile phase: [water(FA)-ACN]; B % 18%-48%, 7 min) to give (S)-3-(5-(4-((1-(4-((1S,2R)-2-((3S,5S,7S)-adamantan-1-yl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (19 mg, 24 μmol, 51% yield, 100% purity) was obtained as a white solid.

LC-MS (ESI+) m/z: 782.3 (M+H)⁺

HPLC: 100%, purity at 220 nm.

SFC: retention time, 3.564 min; Area, 96.797%.

¹H NMR (400 MHZ, DMSO-d₆) δ=10.96 (s, 1H), 8.98 (br s, 1H), 8.22 (br s, 1H), 7.52 (d, J=8.2 Hz, 1H), 7.10-7.02 (m, 2H), 6.98 (br d, J=8.0 Hz, 2H), 6.76 (br d, J=8.3 Hz, 2H), 6.58 (d, J=8.2 Hz, 1H), 6.48 (s, 1H), 6.38 (br d, J=8.1 Hz, 1H), 5.05 (dd, J=4.8, 13.3 Hz, 1H), 4.39-4.28 (m, 1H), 4.25-4.14 (m, 1H), 4.10 (br s, 1H), 3.59 (br d, J=11.6 Hz, 2H), 3.29 (br s, 6H), 3.03-2.65 (m, 4H), 2.58 (br d, J=15.3 Hz, 3H), 2.37 (br dd, J=4.0, 13.4 Hz, 1H), 2.20 (br d, J=6.7 Hz, 2H), 1.95 (br d, J=6.1 Hz, 2H), 1.86-1.73 (m, 6H), 1.72-1.40 (m, 12H), 1.35 (br d, J=11.3 Hz, 3H), 1.22 (br d, J=12.4 Hz, 2H).

### Example 237. Preparation of (I-116) (3.5)-3-[5-[4-[[1-[4-[(1S,2S)-6-hydroxy-2-(3,3,5,5-tetramethylcyclohexyl)tetralin-1-yl]phenyl]-4-piperidyl]methyl]piperazin-1-yl]-1-oxo-isoindolin-2-yl]piperidine-2,6-dione and (I-117) (35)-3-[5-[4-[[1-[4-[(1R,2R)-6-hydroxy-2-(3,3,5,5-tetramethylcyclohexyl)tetralin-1-yl]phenyl]-4-piperidyl]methyl]piperazin-1-yl]-1-oxo-isoindolin-2-yl]piperidine-2,6-dione

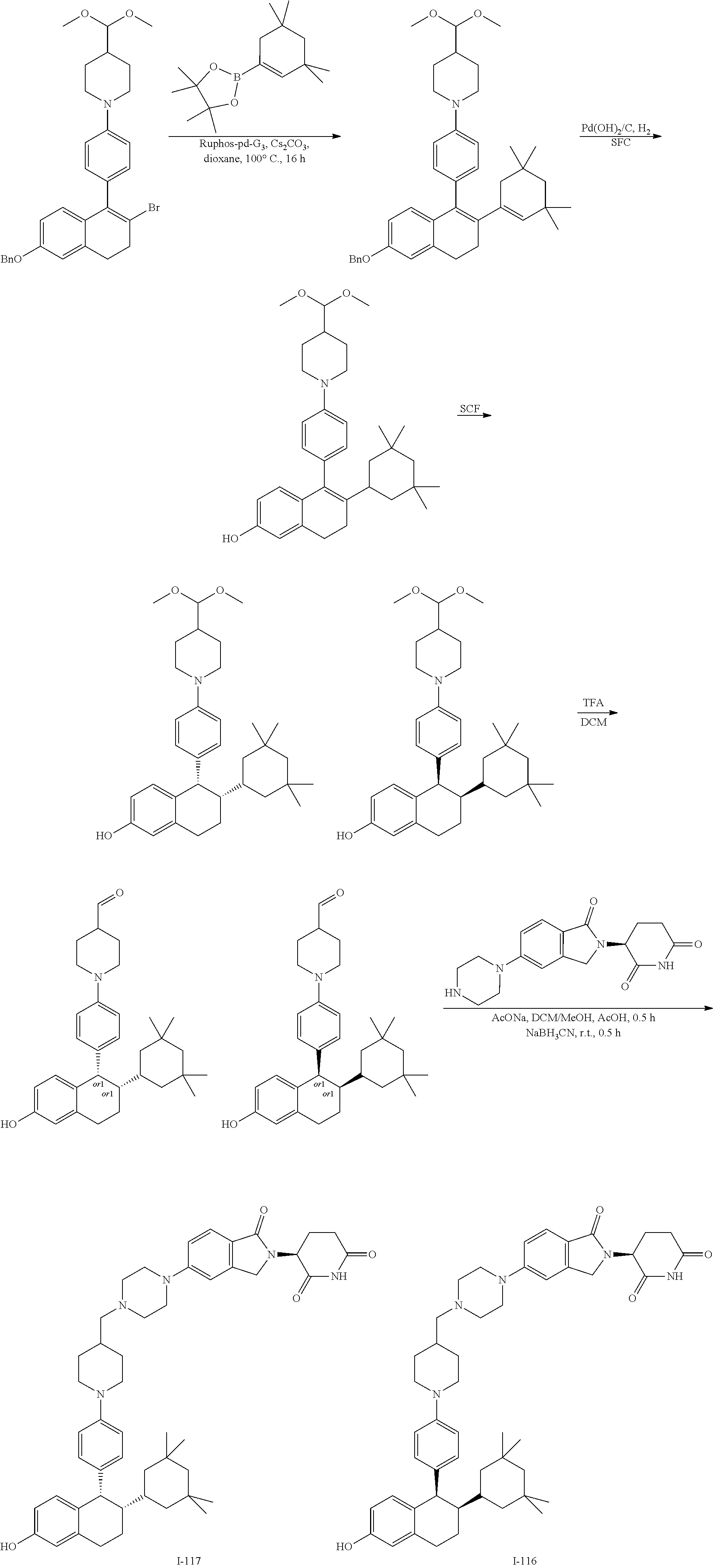

Step 1: A mixture of 1-[4-(6-benzyloxy-2-bromo-3,4-dihydronaphthalen-1-yl)phenyl]-4-(dimethoxymethyl)piperidine (1 g, 1.55 mmol), 4,4,5,5-tetramethyl-2-(3,3,5,5-tetramethylcyclohexen-1-yl)-1,3,2-dioxaborolane (532.26 mg, 2.01 mmol), RuPhos Pd G3 (129.61 mg, 154.97 μmol), Cs₂CO₃ (1.01 g, 3.10 mmol) in dioxane (10 mL) was degassed and purged with N₂ for 3 times, and then the mixture was stirred at 100° C. for 16 h under N₂ atmosphere. The reaction mixture was quenched by water (50 mL) and extracted with EtOAc (60 mL×3). The combined organic layers were washed with brine (100 mL), dried over Na₂SO₄, filtered and concentrated under reduced pressure to give a residue. The residue was purified by flash silica gel chromatography (ethyl acetate in petroleum ether=0% to 10%) to give 1-[4-[6-benzyloxy-2-(3,3,5,5-tetramethylcyclohexen-1-yl)-3,4-dihydronaphthalen-1-yl]phenyl]-4-(dimethoxymethyl)piperidine (500 mg, 48.5% yield) as a yellow solid. LC-MS (ESI⁺) m/z: 606.5 (M+H)⁺.

Step 2: To a solution of Pd(OH)₂/C (3 g, 20% purity) in MeOH (20 mL) and THF (10 mL) was added 1-[4-[6-benzyloxy-2-(3,3,5,5-tetramethylcyclohexen-1-yl)-3,4-dihydronaphthalen-1-yl]phenyl]-4-(dimethoxymethyl)piperidine (400 mg, 600.81 μmol) under N₂. The suspension was degassed under vacuum and purged with H₂ several times. The mixture was stirred under H₂ (50 psi) at 70° C. for 24 h. The reaction mixture was filtered and the filter was concentrated. The residue was purified by flash silica gel chromatography (ethyl acetate in petroleum ether=0% to 30%) to give 1-[4-[4-(dimethoxymethyl)-1-piperidyl]phenyl]-2-(3,3,5,5-tetramethylcyclohexyl)tetralin-6-ol (50 mg, 14.4% yield) as a white solid. LC-MS (ESI⁺) m/z: 520.5 (M+H)⁺.

Step 3: 1-[4-[4-(Dimethoxymethyl)-1-piperidyl]phenyl]-2-(3,3,5,5-tetramethylcyclohexyl)tetralin-6-ol (50 mg, 86.58 μmol) was separated by chiral SFC (column: DAICEL CHIRALCEL OJ (250 mm*30 mm, 10 um); mobile phase: [0.1% NH₃H₂O IPA]; B % 20%-20%, min, peak 1:2.748 min, peak 2:3.019 min) to yield Peak 1 and Peak 2. Peak 1 was concentrated under reduced pressure to yield (1S,2S)-1-[4-[4-(dimethoxymethyl)-1-piperidyl]phenyl]-2-(3,3,5,5-tetramethylcyclohexyl)tetralin-6-ol (10 mg, 21.1% yield, SFC: Rt=2.748) as a white solid. LC-MS (ESI⁺) m/z: 520.5 (M+H)⁺. Peak 2 was concentrated under reduced pressure to yield (1R,2R)-1-[4-[4-(dimethoxymethyl)-1-piperidyl]phenyl]-2-(3,3,5,5-tetramethylcyclohexyl)tetralin-6-ol (10 mg, 21.1% yield, SFC: Rt=3.019) as a white solid. LC-MS (ESI⁺) m/z: 520.5 (M+H)⁺

Step 4: To a solution of (1R,2R)-1-[4-[4-(dimethoxymethyl)-1-piperidyl]phenyl]-2-(3,3,5,5-tetramethylcyclohexyl)tetralin-6-ol (10 mg, 18.66 μmol) in DCM (2 mL) was added TFA (924.00 mg, 8.10 mmol, 0.6 mL). The mixture was stirred at 25° C. for 1 h, followed by lyophilization to give 1-[4-[(1R,2R)-6-hydroxy-2-(3,3,5,5-tetramethylcyclohexyl)tetralin-1-yl]phenyl]piperidine-4-carbaldehyde (10 mg, crude) as a yellow solid LC-MS (ESI⁺) m/z: 474.3 (M+H)⁺

Step 5: To a solution of (1S,2S)-1-[4-[4-(dimethoxymethyl)-1-piperidyl]phenyl]-2-(3,3,5,5-tetramethylcyclohexyl)tetralin-6-ol (10 mg, 18.28 μmol) in DCM (2 mL) was added TFA (924.00 mg, 8.10 mmol, 0.6 mL). The mixture was stirred at 25° C. for 1 h, followed by lyophilization to give 1-[4-[(1S,2S)-6-hydroxy-2-(3,3,5,5-tetramethylcyclohexyl)tetralin-1-yl]phenyl]piperidine-4-carbaldehyde (10 mg, crude) as a yellow solid LC-MS (ESI⁺) m/z: 474.3 (M+H)⁺.

Step 6: To a solution of 1-[4-[(1S,2S)-6-hydroxy-2-(3,3,5,5-tetramethylcyclohexyl)tetralin-1-yl]phenyl]piperidine-4-carbaldehyde (10 mg, 21.11 μmol) in MeOH (1 mL) and DCM (1 mL) was added (3S)-3-(1-oxo-5-piperazin-1-yl-isoindolin-2-yl)piperidine-2,6-dione (10.54 mg, 27.70 μmol, TSOH) and stirred at 25° C. for 0.5 h. Then NaBH(OAc) 3 (4.47 mg, 21.11 μmol) was added and stirred at 25° C. for 0.5 h. The solvent was removed to yield a residue. The residue was purified by preparative HPLC (column: Boston Green ODS 150*30 mm*5 um; mobile phase: [water(FA)-ACN]; B %: 22%-52%, 10 min), followed by lyophilization to yield (3S)-3-[5-[4-[ ]-[4-[(1S,2S)-6-hydroxy-2-(3,3,5,5-tetramethylcyclohexyl)tetralin-1-yl]phenyl]-4-piperidyl]methyl]piperazin-1-yl]-1-oxo-isoindolin-2-yl]piperidine-2,6-dione (1 mg, 6.0% yield) as a white solid. LC-MS (ESI⁺) m/z: 786.6 (M+H)⁺

LCMS: calc. for C₄₉H₆₃N₅O₄: 785.49, found: [M+H]⁺ 786.6.

HPLC: 97.71% purity at 254 nm.

¹HNMR (500 MHz, MeOD-d4): δ 7.67 (d, J=8.4 Hz, 1H), 7.15-7.09 (m, 5H), 7.08-7.05 (m, 1H), 7.04-6.95 (m, 3H), 5.12 (dd, J=5.2, 13.3 Hz, 1H), 4.49-4.38 (m, 2H), 4.02 (s, 1H), 3.75 (d, J=12.4 Hz, 2H), 3.44 (s, 4H), 3.09-3.03 (m, 1H), 2.97-2.87 (m, 1H), 2.80-2.73 (m, 8H), 2.53-2.43 (m, 4H), 2.17 (d, J=13.1 Hz, 1H), 1.96 (d, J=13.0 Hz, 3H), 1.86 (s, 1H), 1.65 (s, 1H), 1.49-1.43 (m, 2H), 1.33-1.29 (m, 3H), 1.19-1.13 (m, 3H), 1.07 (s, 1H), 0.85 (d, J=4.9 Hz, 6H), 0.69 (s, 3H), 0.66 (s, 3H).

Step 7: To a solution of 1-[4-[(1R,2R)-6-hydroxy-2-(3,3,5,5-tetramethylcyclohexyl)tetralin-1-yl]phenyl]piperidine-4-carbaldehyde (10 mg, 21.11 μmol) in MeOH (1 mL) and DCM (1 mL) was added (35)-3-(1-oxo-5-piperazin-1-yl-isoindolin-2-yl)piperidine-2,6-dione (10.54 mg, 27.70 μmol, TSOH) and stirred at 25° C. for 0.5 h. Then NaBH(OAc); (4.47 mg, 21.11 μmol) was added and stirred at 25° C. for 0.5 h. The solvent was removed to yield a residue. The residue was purified by preparative HPLC (column: Boston Green ODS 150*30 mm*5 um; mobile phase: [water(FA)-ACN]; B %: 22%-52%, 10 min), followed by lyophilization to yield (3S)-3-[5-[4-[[1-[4-[(1R,2R)-6-hydroxy-2-(3,3,5,5-tetramethylcyclohexyl)tetralin-1-yl]phenyl]-4-piperidyl]methyl]piperazin-1-yl]-1-oxo-isoindolin-2-yl]piperidine-2,6-dione (1 mg, 6.0% yield) as a white solid. LC-MS (ESI⁺) m/z: 786.6 (M+H)⁺

LCMS: calc. for C₄₉H₆₃N₅O₄:785.49, found: [M+H]⁺ 786.6.

HPLC: 95.70% purity at 254 nm.

¹HNMR (500 MHz, MeOD-d4): δ 7.65 (d, J=8.5 Hz, 1H), 7.13-7.07 (m, 5H), 7.06-7.03 (m, 1H), 7.02-6.95 (m, 3H), 5.10 (dd, J=5.3, 13.4 Hz, 1H), 4.47-4.35 (m, 2H), 4.06-3.93 (m, 1H), 3.73 (d, J=12.7 Hz, 2H), 3.42 (br s, 4H), 3.04 (dd, J=4.8, 16.3 Hz, 1H), 2.96-2.84 (m, 1H), 2.77-2.70 (m, 8H), 2.48-2.41 (m, 1H), 2.52-2.41 (m, 4H), 2.20-2.10 (m, 1H), 1.98-1.85 (m, 4H), 1.69-1.57 (m, 1H), 1.48-1.41 (m, 2H), 1.30 (t, J=12.7 Hz, 3H), 1.18-1.11 (m, 3H), 1.06-1.01 (m, 1H), 0.83 (d, J=4.9 Hz, 6H), 0.67 (s, 3H), 0.65 (s, 3H).

### Example 238. Preparation of (I-105) (S)-3-(5-(4-((1-(4-((1R,2R)-2-(4,4-dimethylcyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione and (I-106) (S)-3-(5-(4-((1-(4-((1S,2S)-2-(4,4-dimethylcyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

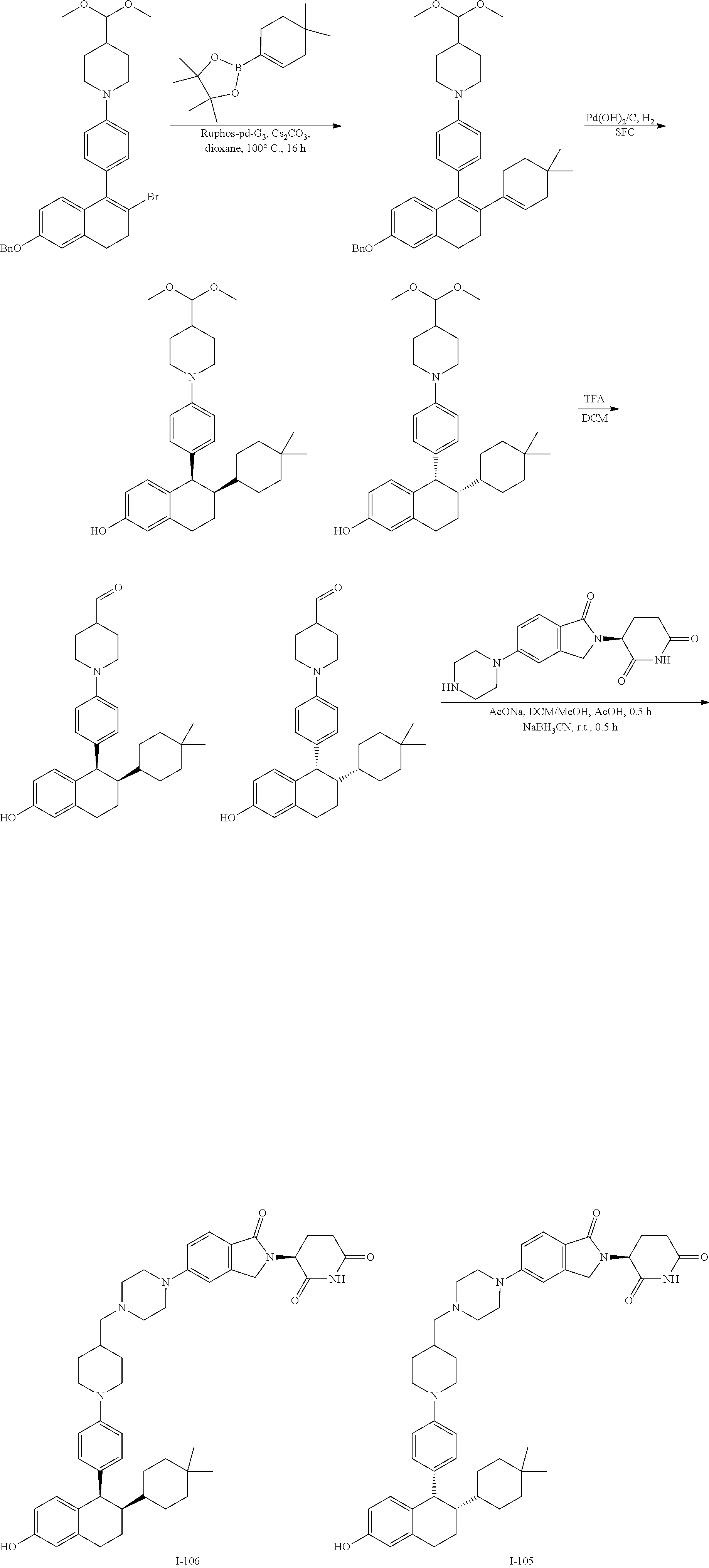

Step 1: A mixture of 1-(4-(6-(benzyloxy)-2-bromo-3,4-dihydronaphthalen-1-yl)phenyl)-4-(dimethoxymethyl)piperidine (1.5 g, 2.32 mmol, 1.0 eq.), 2-(4,4-dimethylcyclohex-1-en-1-yl)-4,4,5,5-tetramethyl-1,3,2-dioxaborolane (658.73 mg, 2.79 mmol, 1.2 eq.), RuPhos Pd G; (194.41 mg, 232.45 μmol, 0.1 eq.), Cs₂CO₃ (1.51 g, 4.65 mmol, 2.0 eq.) and in dioxane (20 mL) was degassed and purged with N₂ for 3 times, and then the mixture was stirred at 100° C. for 16 h under N₂ atmosphere. The reaction mixture was quenched by water (50 mL) and extracted with

EtOAc (60 mL×3). The combined organic layers were washed with brine (100 mL), dried over Na₂SO₄, filtered and concentrated under reduced pressure to give a residue. The residue was purified by preparative HPLC (column: Welch Ultimate XB-NH₂ 250*50*10 um; mobile phase: [Heptane-EtOH (0.1% NH₃H₂O)]; B %: 0%-15%, 9 min) to yield 1-(4-(6-(benzyloxy)-2-(4,4-dimethylcyclohex-1-en-1-yl)-3,4-dihydronaphthalen-1-yl)phenyl)-4-(dimethoxymethyl)piperidine (700 mg, 46.4% yield) as a yellow solid. LC-MS (ESI⁺) m/z: 578.4 (M+H)⁺.

Step 2: To a suspension of Pd(OH)₂/C (1.60 g, 20% purity) in MeOH (30 mL) and THF (10 mL) was added 1-(4-(6-(benzyloxy)-2-(4,4-dimethylcyclohex-1-en-1-yl)-3,4-dihydronaphthalen-1-yl)phenyl)-4-(dimethoxymethyl)piperidine (600 mg, 924.21 μmol, 1.0 eq.) under N₂. The suspension was degassed under vacuum and purged with H₂ several times. The mixture was stirred under H₂ (50 psi) at 70° C. for 24 h. The reaction mixture was filtered and the filter was concentrated. The residue was purified by flash silica gel chromatography (ethyl acetate in petroleum ether=0% to 40%) to give a product which was separated by chiral SFC (column: DAICEL CHIRALPAK AD (250 mm*30 mm, 10 um); mobile phase: [0.1% NH₃H₂O MEOH]; B %: 40%-40%, min, peak 1:1.528 min, peak 2:2.046 min) to yield Peak 1 and Peak 2. Peak 1 was concentrated under reduced pressure to yield (5R,6R)-5-(4-(4-(dimethoxymethyl)piperidin-1-yl)phenyl)-6-(4,4-dimethylcyclohexyl)-5,6,7,8-tetrahydronaphthalen-2-ol (100 mg, 28.3% yield, SFC: Rt=1.528 min) as a white solid. LC-MS (ESI⁺) m/z: 492.4 (M+H)⁺. Peak 2 was concentrated under reduced pressure to yield (5S,6S)-5-(4-(4-(dimethoxymethyl)piperidin-1-yl)phenyl)-6-(4,4-dimethylcyclohexyl)-5,6,7,8-tetrahydronaphthalen-2-ol (100 mg, 29.5% yield, SFC: Rt=2.046 min) as a white solid. LC-MS (ESI⁺) m/z: 492.4 (M+H)⁺.

Step 3: To a solution of (5R,6R)-5-(4-(4-(dimethoxymethyl)piperidin-1-yl)phenyl)-6-(4,4-dimethylcyclohexyl)-5,6,7,8-tetrahydronaphthalen-2-ol (50 mg, 95.59 μmol, 1.0 eq.) in DCM (2 mL) was added TFA (924.00 mg, 8.10 mmol, 0.6 mL). The mixture was stirred at 25° C. for 3 h, followed by lyophilization to give 1-(4-((1R,2R)-2-(4,4-dimethylcyclohexyl)-6-hydroxy-1,2,3,4-tetrahydronaphthalen-1-yl)phenyl)piperidine-4-carbaldehyde (42 mg, crude) as a yellow solid. LC-MS (ESI⁺) m/z: 446.3 (M+H)⁺.

Step 4: To a solution of (5S,6S)-5-(4-(4-(dimethoxymethyl)piperidin-1-yl)phenyl)-6-(4,4-dimethylcyclohexyl)-5,6,7,8-tetrahydronaphthalen-2-ol (50 mg, 95.65 μmol, 1.0 eq.) in DCM (2 mL) was added TFA (924.00 mg, 8.10 mmol, 0.6 mL). The mixture was stirred at 25° C. for 3 h. followed by lyophilization to give 1-(4-((1S,2S)-2-(4,4-dimethylcyclohexyl)-6-hydroxy-1,2,3,4-tetrahydronaphthalen-1-yl)phenyl)piperidine-4-carbaldehyde (40 mg, crude) as a yellow solid. LC-MS (ESI⁺) m/z: 446.3 (M+H)⁺.

Step 5: To a solution of (S)-3-(1-oxo-5-(piperazin-1-yl)isoindolin-2-yl)piperidine-2,6-dione (30.95 mg, 94.25 μmol, benzenesulfonic acid, 1.0 eq.) in DCM (1 mL) and MeOH (1 mL) was added 1-(4-((1R,2R)-2-(4,4-dimethylcyclohexyl)-6-hydroxy-1,2,3,4-tetrahydronaphthalen-1-yl)phenyl)piperidine-4-carbaldehyde (42 mg, 94.25 μmol, 1.0 eq.) and stirred at 25° C. for 0.5 h. Then NaBH(OAc) 3 (19.97 mg, 94.25 μmol, 1.0 eq.) was added and stirred at 25° C. for 0.5 b. The solvent was removed to yield a residue. The residue was purified by preparative HPLC (column: Welch Xtimate C18 150*25 mm*5 um; mobile phase: [water(FA)-ACN]; B %: 23%-53%, 11 min), followed by lyophilization to yield (S)-3-(5-(4-((1-(4-((1R,2R)-2-(4,4-dimethylcyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (17.8 mg, 23.48 μmol, 24.9% yield) as a white solid. LC-MS (ESI⁺) m/z: 758.5 (M+H)⁺

LCMS: calc. for C₄₇H₅₉N₅O₄:757.46, found: [M+H]⁺ 758.5.

HPLC: 100.00% purity at 254 nm.

¹HNMR (500 MHz, MeOD-d4): δ 7.65 (d, J=8.4 Hz, 1H), 7.16-7.05 (m, 6H), 7.01 (d, J=8.1 Hz, 1H), 6.98-6.92 (m, 1H), 6.95 (dt, J=2.7, 10.4 Hz, 1H), 5.10 (dd, J=5.2, 13.3 Hz, 1H), 4.48-4.35 (m, 2H), 4.01-3.95 (m, 1H), 3.76 (d, J=12.2 Hz, 2H), 3.44-3.39 (m, 4H), 3.03 (dd, J=4.7, 15.5 Hz, 1H), 2.95-2.86 (m, 1H), 2.82-2.67 (m, 9H), 2.49-2.39 (m, 4H), 2.36-2.26 (m, 1H), 2.23-2.11 (m, 2H), 1.97 (d, J=12.2 Hz, 2H), 1.91-1.78 (m, 2H), 1.65-1.55 (m, 3H), 1.47-1.39 (m, 4H), 1.36-1.31 (m, 2H), 0.96 (s, 3H), 0.82 (s, 3H).

Step 6: To a solution of (S)-3-(1-oxo-5-(piperazin-1-yl)isoindolin-2-yl)piperidine-2,6-dione (40 mg, 121.82 μmol, benzenesulfonic acid, 1.35 eq.) in DCM (1 mL) and MeOH (1 mL) was added 1-(4-((1S,2S)-2-(4,4-dimethylcyclohexyl)-6-hydroxy-1,2,3,4-tetrahydronaphthalen-1-yl)phenyl)piperidine-4-carbaldehyde (40 mg, 89.76 μmol, 1.0 eq.) and stirred at 25° C. for 0.5 h. Then NaBH(OAc) 3 (19.02 mg, 89.76 μmol, 1.0 eq.) and stirred at 25° C. for 0.5 h. The solvent was removed to yield a residue. The residue was purified by preparative HPLC (column: Welch Xtimate C18 150*25 mm*5 um; mobile phase: [water(FA)-ACN]; B %: 23%-53%, 11 min), followed by lyophilization to yield (S)-3-(5-(4-((1-(4-((1S,2S)-2-(4,4-dimethylcyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (12.5 mg, 15.99 μmol, 17.8% yield) as a white solid. LC-MS (ESI⁺) m/z: 758.6 (M+H)⁺

LCMS: calc. for C₄₇H₅₉N₅O₄: 757.46, found: [M+H]⁺ 758.6.

HPLC: 96.95% purity at 254 nm.

¹HNMR (500 MHz, MeOD-d4): δ 7.65 (d, J=8.4 Hz, 1H), 7.16-7.05 (m, 6H), 7.01 (d, J=8.1 Hz, 1H), 6.95 (dt, J=2.7, 10.4 Hz, 2H), 5.11 (dd, J=5.0, 13.3 Hz, 1H), 4.47-4.35 (m, 2H), 4.02-3.94 (m, 1H), 3.76 (d, J=12.2 Hz, 2H), 3.42 (s, 4H), 3.03 (dd, J=4.6, 15.9 Hz, 1H), 2.96-2.86 (m, 1H), 2.81-2.67 (m, 9H), 2.49-2.40 (m, 4H), 2.35-2.26 (m, 1H), 2.22-2.12 (m, 2H), 1.97 (d, J=12.8 Hz, 2H), 1.85 (d, J=15.1 Hz, 2H), 1.64-1.55 (m, 3H), 1.44 (d, J=12.4 Hz, 4H), 1.35-1.31 (m, 2H), 0.96 (s, 3H), 0.82 (s, 3H).

Stereochemistry of the two compounds was arbitrarily assigned

### Example 239. Preparation of (I-55) (S)-3-(5-(4-((1-(4-((1S,2S)-2-(4,4-difluorocyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

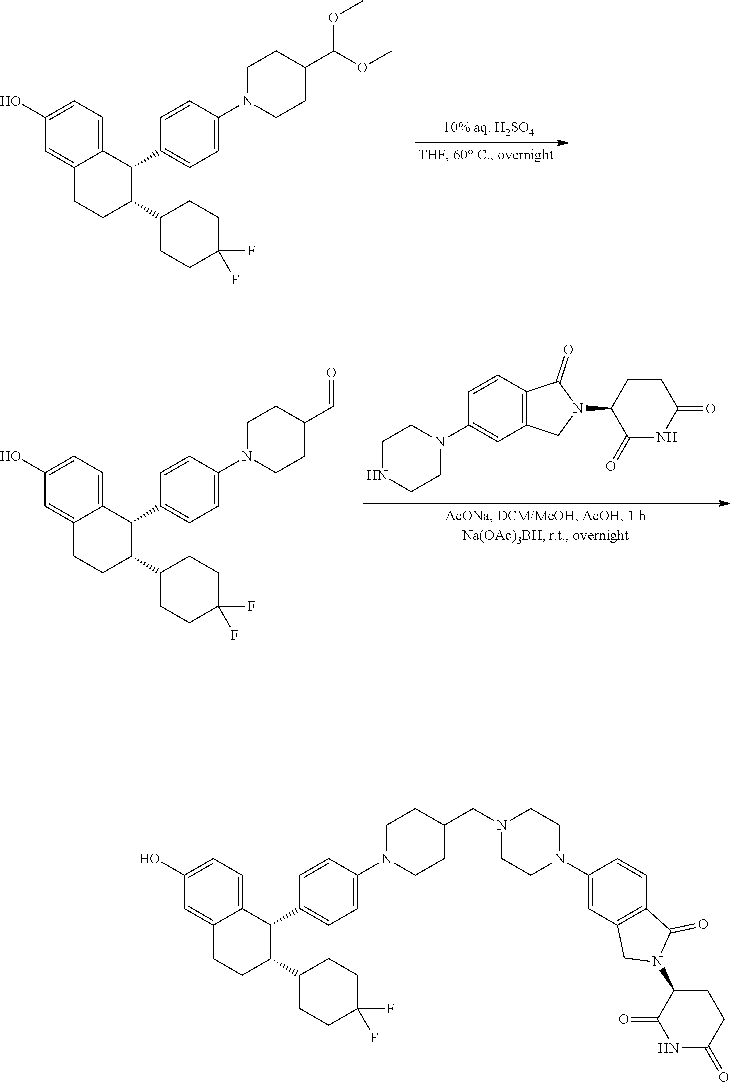 [SMILES: COC(OC)C1CCN(c2ccc([C@H]3c4ccc(O)cc4CC[C@H]3C3CCC(F)(F)CC3)cc2)CC1 ; O=CC1CCN(c2ccc([C@H]3c4ccc(O)cc4CC[C@H]3C3CCC(F)(F)CC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7C7CCC(F)(F)CC7)cc6)CC5)CC4)ccc3C2=O)C(=O)N1]

Step 1: A mixture of (5S,6S)-6-(4,4-difluorocyclohexyl)-5-(4-(4-(dimethoxymethyl)piperidin-1-yl)phenyl)-5,6,7,8-tetrahydronaphthalen-2-ol (90 mg, 1 Eq, 0.17 mmol) in 10% H₂SO₄ (1.5 mL) and THF (1.5 mL), then the mixture was stirred at 60° C. for 16 hour. The solvent was removed under vacuum. The residue was dissolved in water (20 mL) and washed by ethyl acetate (20 mL). The aqueous solution was added NaHCO₃ to adjust pH=6. The combined organic layer was dried over Na₂SO₄, filtered and concentrated under vacuum to give 1-(4-((1S,2S)-2-(4,4-difluorocyclohexyl)-6-hydroxy-1,2,3,4-tetrahydronaphthalen-1-yl)phenyl)piperidine-4-carbaldehyde (80 mg, 0.18 mmol, 100%) as a yellow oil. LC-MS (ESI+) m/z: 454.1 (M+H)+.

Step 2: A mixture of 1-(4-(1S,2S)-2-(4,4-difluorocyclohexyl)-6-hydroxy-1,2,3,4-tetrahydronaphthalen-1-yl)phenyl)piperidine-4-carbaldehyde (80 mg, 1 Eq, 0.18 mmol), (R)-3-(1-oxo-5-(piperazin-1-yl)isoindolin-2-yl)piperidine-2,6-dione, Tosylic acid (0.11 g, 1.3 Eq, 0.23 mmol) and Sodium acetate (72 mg, 5 Eq, 0.88 mmol) and Acetic acid (32 mg, 30 μL, 3 Eq. 0.53 mmol) in DCM (2 mL) and MeOH (2 mL) at 25° C. for 1 hour, then Sodium triacetoxyborohydride (75 mg, 2 Eq, 0.35 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 21%-51%, 7 min). The aqueous phase was lyophilized to dryness to give (S)-3-(5-(4-((1-(4-((1S,2S)-2-(4,4-difluorocyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (54.7 mg, 68.5 μmol, 39%, 95.85% Purity) as a white solid.

LC-MS (ESI+) m/z: 766.2 (M+H)+

HPLC: 95.850%, purity at 220 nm.

SFC: retention time, 2.655 min; Area, 85.695%; Stereochemistry was arbitrarily assigned ¹H NMR (400 MHZ, DMSO-d₆) δ=11.00-10.90 (m, 1H), 9.10-8.96 (m, 1H), 8.18-8.14 (m, 0.401H), 7.58-7.45 (m, 1H), 7.11-7.01 (m, 2H), 6.87-6.72 (m, 4H), 6.67-6.57 (m, 1H), 6.54-6.47 (m, 1H), 6.46-6.38 (m, 1H), 5.11-5.01 (m, 1H), 4.39-4.28 (m, 1H), 4.26-4.16 (m, 1H), 4.11-4.02 (m, 1H), 3.67-3.54 (m, 2H), 3.43-3.31 (m, 6H), 2.99-2.82 (m, 2H), 2.80-2.69 (m, 1H), 2.64-2.52 (m, 5H), 2.37-2.30 (m, 1H), 2.26-2.18 (m, 2H), 2.17-2.07 (m, 1H), 2.06-1.92 (m, 2H), 1.90-1.76 (m, 3H), 1.74-1.61 (m, 4H), 1.59-1.43 (m, 2H), 1.36-1.12 (m, 5H), 1.08-0.96 (m, 1H).

### Example 240. Preparation of (I-54) (R)-3-(5-(4-((1-(4-((1R,2R)-2-(4,4-difluorocyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

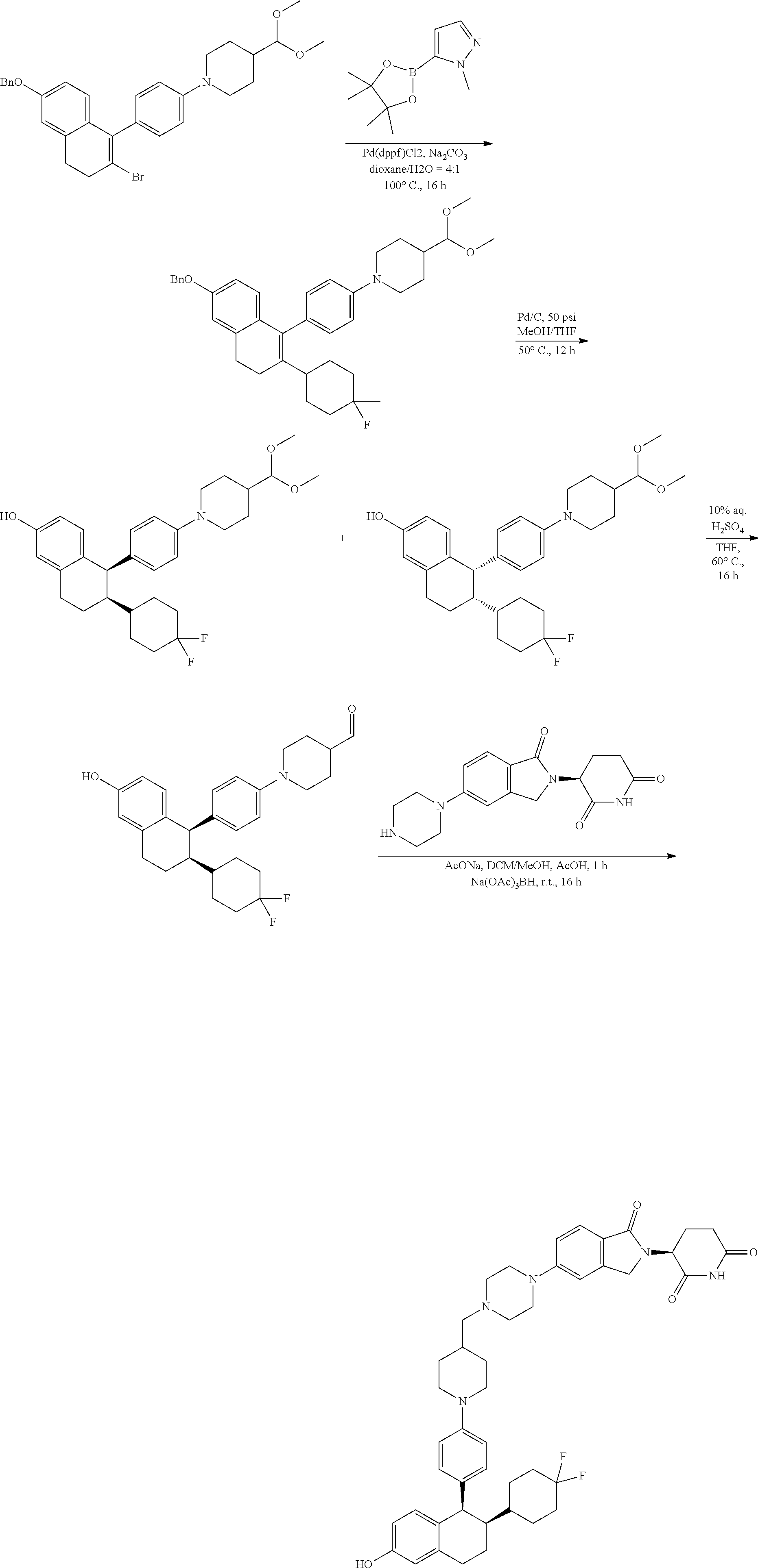

Step 1: A mixture of 1-(4-(6-(benzyloxy)-2-bromo-3,4-dihydronaphthalen-1-yl)phenyl)-4-(dimethoxymethyl)piperidine (1 g, 1 Eq, 2 mmol), 2-(4,4-difluorocyclohex-1-enyl)-4,4,5,5-tetramethyl-1,3,2-dioxaborolane (0.5 g, 1.2 Eq, 2 mmol), Na₂CO₃ (0.6 g, 3 Eq, 5 mmol) and [1,1′-Bis(diphenylphosphino)ferrocene]dichloropalladium(II) complex With Dichloromethane (0.1 g, 0.1 Eq, 0.2 mmol) in Dioxane (12 mL) and H₂O (3.0 mL) at r.t. N₂ was bubbled into the mixture for 5 min. The reaction mixture was heated at 100° C. for 16 hour. Then the reaction was cooled to room temperature. The mixture was treated with H₂O (50 mL), extracted with EtOAc (50 mL). The combined extracts was dried over anhydrous Na₂SO₄, filtered and concentrated to dryness in vacuo to give a residue. The residue was subjected to column chromatography over silica gel (gradient elution: 0˜20% ethyl acetate in petroleum ether). The pure fractions were collected and concentrated to dryness in vacuo to give 1-(4-(6-(benzyloxy)-2-(4,4-difluorocyclohex-1-en-1-yl)-3,4-dihydronaphthalen-1-yl)phenyl)-4-(dimethoxymethyl)piperidine (850 mg, 1.4 mmol, 80%, 97% Purity) as a white solid. LC-MS (ESI⁺) m/z: 586.2 (M+H)⁺.

Step 2: A mixture of 1-(4-(6-(benzyloxy)-2-(4,4-difluorocyclohex-1-en-1-yl)-3,4-dihydronaphthalen-1-yl)phenyl)-4-(dimethoxymethyl)piperidine (750 mg, 1 Eq, 1.24 mmol), Pd/C (800 mg, 10% Wt, 0.605 Eq, 752 μmol), H2 (2.51 mg, 1 Eq, 1.24 mmol) in MeOH (3 mL) and THF (3 mL). The suspension was degassed and purged with H₂ for 3 times. The mixture was stirred under H₂ (50 psi) at 50° C. for 24 hour. Then the reaction was cooled to room temperature. The reaction was filtered and concentrated to dryness in vacuo to give a white solid. The white solid was subjected by SFC: Column: DAICEL CHIRALCEL OD (250 mm*30 mm, 10 um); Mobile phase: A: Supercritical CO₂, B: MeOH (0.1% NH₃H₂O), A:B=65:35 at 80 mL/min. The aqueous phase was lyophilized to dryness to give the product (5S,6S)-6-(4,4-difluorocyclohexyl)-5-(4-(4-(dimethoxymethyl)piperidin-1-yl)phenyl)-5,6,7,8-tetrahydronaphthalen-2-ol (90 mg, 0.18 mmol, 15%, 100% Purity) and (5R,6R)-6-(4,4-difluorocyclohexyl)-5-(4-(4-(dimethoxymethyl)piperidin-1-yl)phenyl)-5,6,7,8-tetrahydronaphthalen-2-ol (90 mg, 0.17 mmol, 14%, 95% Purity) as a yellow solid. LC-MS (ESI⁺) m/z: 500.1 (M+H)⁺

Step 3: A mixture of (5R,6R)-6-(4,4-difluorocyclohexyl)-5-(4-(4-(dimethoxymethyl)piperidin-1-yl)phenyl)-5,6,7,8-tetrahydronaphthalen-2-ol (90 mg, 1 Eq, 0.18 mmol) in 10% H₂SO₄ (1.5 mL) and THF (1.5 mL), then the mixture was stirred at 60° C. for 16 hour. The solvent was removed under vacuum. The residue was dissolved in water (20 mL) and washed by ethyl acetate (20 mL). The aqueous solution was added NaHCO₃ to adjust pH=6. The combined organic layer was dried over Na₂SO₄, filtered and concentrated under vacuum to give 1-(4-((1R,2R)-2-(4,4-difluorocyclohexyl)-6-hydroxy-1,2,3,4-tetrahydronaphthalen-1-yl)phenyl)piperidine-4-carbaldehyde (80 mg, 0.18 mmol, 98%) as a yellow oil. LC-MS (ESI+) m/z: 454.2 (M+H)+

Step 4: A mixture of 1-(4-((1R,2R)-2-(4,4-difluorocyclohexyl)-6-hydroxy-1,2,3,4-tetrahydronaphthalen-1-yl)phenyl)piperidine-4-carbaldehyde (80 mg, 1 Eq, 0.18 mmol), (R)-3-(1-oxo-5-(piperazin-1-yl)isoindolin-2-yl)piperidine-2,6-dione, Tosylic acid (0.11 g, 1.3 Eq, 0.23 mmol) and Sodium acetate (72 mg, 5 Eq, 0.88 mmol) and Acetic acid (32 mg, 30 μL, 3 Eq, 0.53 mmol) in DCM (2 mL) and MeOH (2 mL) at 25° C. for 1 hour, then Sodium triacetoxyborohydride (75 mg, 2 Eq, 0.35 mmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 21%-51%, 7 min). The aqueous phase was lyophilized to dryness to give (R)-3-(5-(4-((1-(4-((1R,2R)-2-(4,4-difluorocyclohexyl)-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (66.8 mg, 83.8 μmol, 48% yield, 96.078% purity) as a white solid.

LC-MS (ESI+) m/z: 766.2 (M+H)⁺

HPLC: 96.078%, purity at 220 nm.

SFC: retention time, 3.063 min; Area, 87.591%; Stereochemistry was arbitrarily assigned.

¹H NMR (400 MHZ, DMSO-d₆) δ=11.00-10.91 (m, 1H), 9.07-8.97 (m, 1H), 8.19-8.08 (m, 0.583H), 7.57-7.50 (m, 1H), 7.10-7.01 (m, 2H), 6.86-6.73 (m, 4H), 6.65-6.59 (m, 1H), 6.54-6.48 (m, 1H), 6.45-6.39 (m, 1H), 5.10-5.01 (m, 1H), 4.39-4.28 (m, 1H), 4.26-4.16 (m, 1H), 4.09-4.02 (m, 1H), 3.66-3.56 (m, 2H), 3.47-3.31 (m, 6H), 2.97-2.82 (m, 2H), 2.79-2.69 (m, 1H), 2.63-2.53 (m, 5H), 2.38-2.30 (m, 1H), 2.27-2.18 (m, 2H), 2.17-2.07 (m, 1H), 2.06-1.92 (m, 2H), 1.90-1.75 (m, 3H), 1.75-1.61 (m, 4H), 1.60-1.43 (m, 2H), 1.35-1.11 (m, 5H), 1.09-0.96 (m, 1H)

### Example 241. Preparation of (I-53) (R)-3-(5-(4-((1-(4-((1S,2S)-2-cyclopentyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

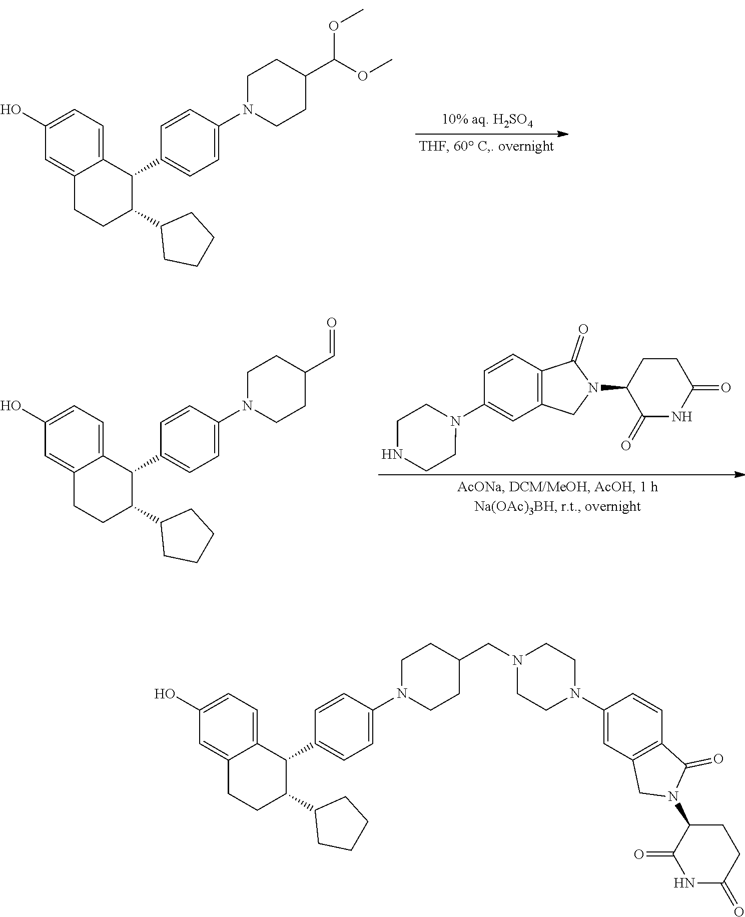

Step 1: A mixture of (5S,6S)-6-cyclopentyl-5-(4-(4-(dimethoxymethyl)piperidin-1-yl)phenyl)-5,6,7,8-tetrahydronaphthalen-2-ol (120 mg, 1 Eq, 265 μmol) in 10% H₂SO₄ (1.5 mL) and THF (1.5 mL), then the mixture was stirred at 60° C. for 16 hour. The solvent was removed under vacuum. The residue was dissolved in water (20 mL) and washed by ethyl acetate (20 mL). The aqueous solution was added NaHCO₃ to adjust pH=6. The combined organic layer was dried over Na₂SO₄, filtered and concentrated under vacuum to give 1-(4-((1S,2S)-2-cyclopentyl-6-hydroxy-1,2,3,4-tetrahydronaphthalen-1-yl)phenyl)piperidine-4-carbaldehyde (100 mg, 0.248 mmol, 93.4%) as a yellow oil. LC-MS (ESI+) m/z; 422.1 (M+H)+.

Step 2: A mixture of 1-(4-((1S,2S)-2-cyclopentyl-6-hydroxy-1,2,3,4-tetrahydronaphthalen-1-yl)phenyl)piperidine-4-carbaldehyde (100 mg, 1 Eq, 248 μmol), (R)-3-(1-oxo-5-(piperazin-1-yl)isoindolin-2-yl)piperidine-2,6-dione, TsOH acid (161 mg, 1.3 Eq, 322 μmol) and Sodium acetate (102 mg, 5 Eq, 1.24 mmol) and Acetic acid (44.6 mg, 42.8 uL, 3 Eq, 743 μmol) in DCM (2 mL) and MeOH (2 mL) at 25° C. for 1 hour, then Sodium triacetoxyborohydride (105 mg, 2 Eq, 496 μmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 31%-61%, 7 min). The aqueous phase was lyophilized to dryness to give (R)-3-(5-(4-((1-(4-((1S,2S)-2-cyclopentyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (59.4 mg, 77.8 μmol, 31.4%, 93.724% Purity) as a pink solid.

LC-MS (ESI+) m/z: 716.3 (M+H)+

HPLC: 93.724%, purity at 220 nm.

SFC: retention time, 3.024 min; Area, 87.334%; Stereochemistry was arbitrarily assigned ¹H NMR (400 MHZ, DMSO-d₆) δ=10.99-10.91 (m, 1H), 9.16-8.86 (m, 1H), 8.18-8.13 (m, 0.504H), 7.49 (s, 1H), 7.12-7.01 (m, 2H), 6.85-6.74 (m, 4H), 6.65-6.58 (m, 1H), 6.53-6.48 (m, 1H), 6.45-6.39 (m, 1H), 5.11-5.00 (m, 1H), 4.39-4.28 (m, 1H), 4.26-4.12 (m, 1H), 3.98-3.90 (m, 1H), 3.63-3.55 (m, 2H), 3.42-3.24 (m, 10H), 2.96-2.66 (m, 4H), 2.27-2.17 (m, 2H), 2.08-1.91 (m, 2H), 1.86-1.74 (m, 2H), 1.74-1.42 (m, 9H), 1.36-1.16 (m, 5H), 1.12-0.98 (m, 1H).

### Example 242. Preparation of (I-52) (R)-3-(5-(4-((1-(4-((1R,2R)-2-cyclopentyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione

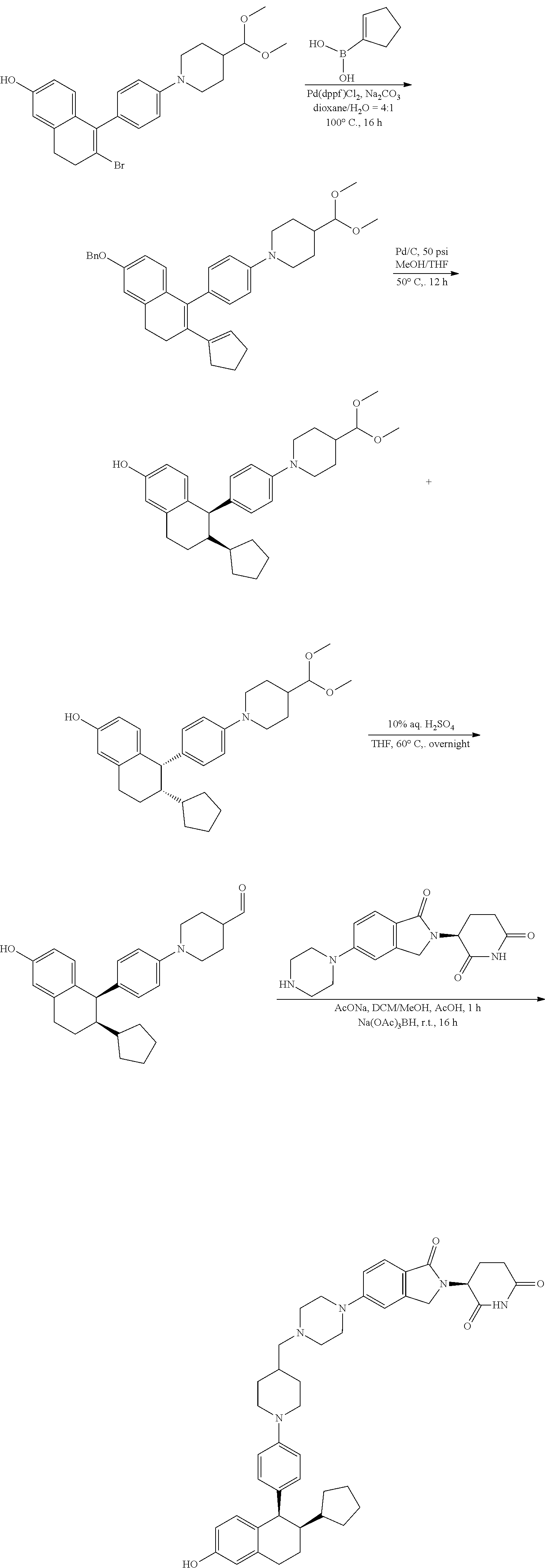

Step 1: A mixture of 1-(4-(6-(benzyloxy)-2-bromo-3,4-dihydronaphthalen-1-yl)phenyl)-4-(dimethoxymethyl)piperidine (1 g, 1 Eq, 2 mmol), cyclopent-1-en-1-ylboronic acid (0.2 g, 1.2 Eq. 2 mmol), Na₂CO₃ (0.6 g, 3 Eq. 5 mmol) and Pd(dppf)Cl₂ (0.1 g, 0.1 Eq, 0.2 mmol) in dioxane (12 mL) and H₂O (3.0 mL) at r.t. N₂ was bubbled into the mixture for 5 min. The reaction mixture was heated at 100° C. for 16 hour. Then the reaction was cooled to room temperature. The mixture was treated with H₂O (50 mL), extracted with EtOAc (50 mL). The combined extracts was dried over anhydrous Na₂SO₄, filtered and concentrated to dryness in vacuo to give a residue. The residue was subjected to column chromatography over silica gel (gradient elution: 0˜20% ethyl acetate in petroleum ether). The pure fractions were collected and concentrated to dryness in vacuo to give 1-(4-(6-(benzyloxy)-2-(cyclopent-1-en-1-yl)-3,4-dihydronaphthalen-1-yl)phenyl)-4-(dimethoxymethyl)piperidine (760 mg, 1.3 mmol, 70%, 93% Purity) as a yellow oil. LC-MS (ESI⁺) m/z: 536.1 (M+H)⁺.

Step 2: A mixture of 1-(4-(6-(benzyloxy)-2-(cyclopent-1-en-1-yl)-3,4-dihydronaphthalen-1-yl)phenyl)-4-(dimethoxymethyl)piperidine (650 mg, 93% Wt, 1 Eq, 1.13 mmol), Pd/C (650 mg, 10% Wt, 0.541 Eq, 611 μmol), H2 (2.28 mg, 1 Eq, 1.13 mmol) in THF (3 mL) and MeOH (3 mL) was degassed and purged with H₂ for 3 times and then the mixture was stirred at 50° C. for 12 hour under H₂ (50 psi) atmosphere. The reaction was filtered and concentrated to dryness in vacuo to give a white solid. The white solid was subjected by SFC: Column: DAICEL CHIRALCEL OD (250 mm*30 mm, 10 um); Mobile phase: A: Supercritical CO₂, B: MeOH (0.1% NH₃H₂O), A:B=70:30 at 80 mL/min. The aqueous phase was lyophilized to dryness to give (5S,6S)-6-cyclopentyl-5-(4-(4-(dimethoxymethyl)piperidin-1-yl)phenyl)-5,6,7,8-tetrahydronaphthalen-2-ol (120 mg, 261 μmol, 23.1%, 97.781% purity) and (5R,6R)-6-cyclopentyl-5-(4-(4-(dimethoxymethyl)piperidin-1-yl)phenyl)-5,6,7,8-tetrahydronaphthalen-2-ol (120 mg, 265 μmol, 23.5%, 99.370% purity) as a yellow oil. LC-MS (ESI⁺) m/z: 450.2 (M+H)⁺.

Step 3: A mixture of (5R,6R)-6-cyclopentyl-5-(4-(4-(dimethoxymethyl)piperidin-1-yl)phenyl)-5,6,7,8-tetrahydronaphthalen-2-ol (120 mg, 1 Eq. 261 μmol) in 10% H₂SO₄ (1.5 mL) and THE (1.5 mL), then the mixture was stirred at 60° C. for 16 hour. The solvent was removed under vacuum. The residue was dissolved in water (20 mL) and washed by ethyl acetate (20 mL). The aqueous solution was added NaHCO₃ to adjust pH=6. The combined organic layer was dried over Na₂SO₄, filtered and concentrated under vacuum to give 1-(4-((1R,2R)-6-hydroxy-2-(1-methyl-1H-pyrazol-5-yl)-1,2,3,4-tetrahydronaphthalen-1-yl)phenyl)piperidine-4-carbaldehyde (100 mg, 0.248 mmol, 93.4%) as a yellow oil. LC-MS (ESI+) m/z: 422.1 (M+H)+

Step 4: A mixture of 1-(4-((1R,2R)-2-cyclopentyl-6-hydroxy-1,2,3,4-tetrahydronaphthalen-1-yl)phenyl)piperidine-4-carbaldehyde (100 mg, 1 Eq, 248 μmol), (R)-3-(1-oxo-5-(piperazin-1-yl)isoindolin-2-yl)piperidine-2,6-dione, TsOH acid (161 mg, 1.3 Eq, 322 μmol) and Sodium acetate (102 mg, 5 Eq, 1.24 mmol) and Acetic acid (44.6 mg, 42.8 μL, 3 Eq, 743 μmol) in DCM (2 mL) and MeOH (2 mL) at 25° C. for 1 hour, then Sodium triacetoxyborohydride (105 mg, 2 Eq. 496 μmol) was added to the mixture and was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase:

[water(FA)-ACN]; B %: 26%-56%, 7 min). The aqueous phase was lyophilized to dryness to give (R)-3-(5-(4-((1-(4-((1R,2R)-2-cyclopentyl-6-hydroxy-1,2,3,4-tetrahydronaphthalen-1-yl)phenyl)piperidin-4-yl)methyl)piperazin-1-yl)-1-oxoisoindolin-2-yl)piperidine-2,6-dione (80.8 mg, 109 μmol, 44.2%, 96.954% Purity) as a pink solid.

LC-MS (ESI+) m/z: 716.3 (M+H)+

HPLC: 96.954%, purity at 220 nm.

SFC: retention time, 3.039 min; Arca, 89.199%; Stereochemistry was arbitrarily assigned.

¹H NMR (400 MHZ, DMSO-d₆) δ=10.98-10.91 (m, 1H), 9.06-8.96 (m, 1H), 8.18-8.13 (m, 0.230H), 7.55-7.50 (m, 1H), 7.10-7.02 (m, 2H), 6.88-6.73 (m, 4H), 6.66-6.58 (m, 1H), 6.53-6.48 (m, 1H), 6.45-6.39 (m, 1H), 5.11-5.00 (m, 1H), 4.38-4.27 (m, 1H), 4.26-4.16 (m, 1H), 3.98-3.90 (m, 1H), 3.66-3.54 (m, 2H), 3.42-3.22 (m, 11H), 2.95-2.69 (m, 3H), 2.27-2.18 (m, 2H), 2.07-1.91 (m, 2H), 1.85-1.75 (m, 2H), 1.74-1.42 (m, 9H), 1.35-1.15 (m, 5H), 1.10-0.97 (m, 1H)

### Example 243. Preparation of (I-49) (5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalene-2-carbonitrile

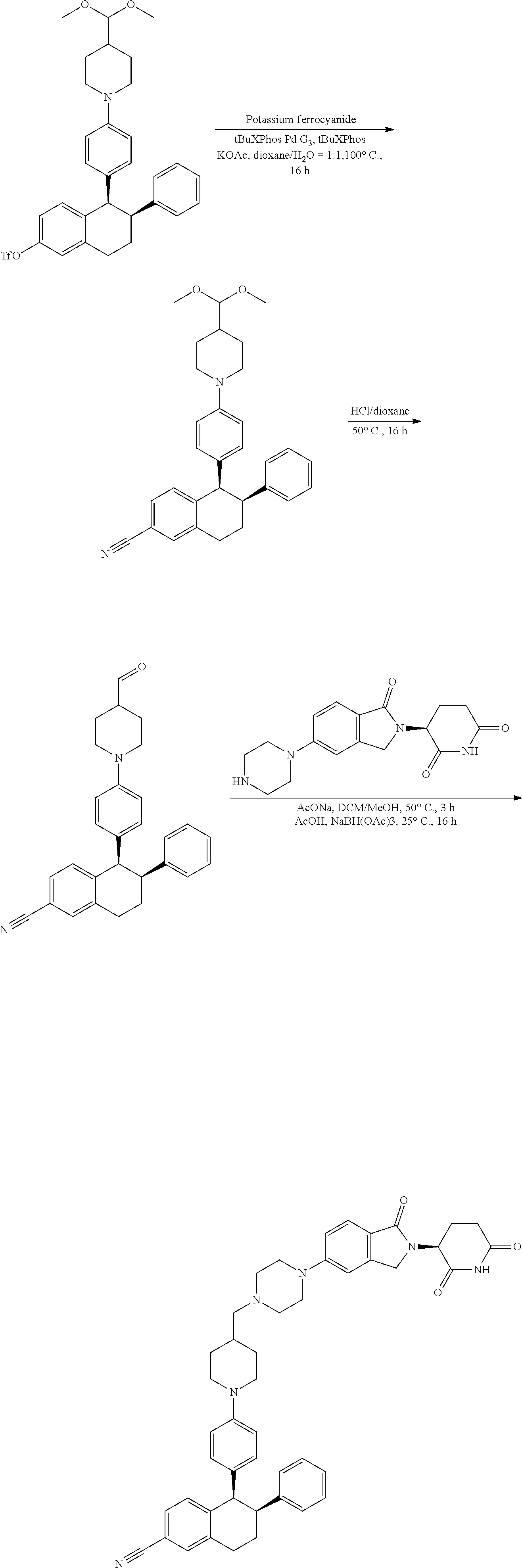 [SMILES: COC(OC)C1CCN(c2ccc([C@@H]3c4ccc(OS(=O)(=O)C(F)(F)F)cc4CC[C@@H]3c3ccccc3)cc2)CC1 ; COC(OC)C1CCN(c2ccc([C@@H]3c4ccc(C#N)cc4CC[C@@H]3c3ccccc3)cc2)CC1 ; N#Cc1ccc2c(c1)CC[C@H](c1ccccc1)[C@@H]2c1ccc(N2CCC(C=O)CC2)cc1 ; O=C1CC[C@H](N2Cc3cc(N4CCNCC4)ccc3C2=O)C(=O)N1 ; N#Cc1ccc2c(c1)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1]

Step 1: A 40 mL thread was vial equipped with magnetic stirrer. (5R,6S)-5-(4-(4-(dimethoxymethyl)piperidin-1-yl)phenyl)-6-phenyl-5,6,7,8-tetrahydronaphthalen-2-yl trifluoromethanesulfonate (500 mg, 848 μmol), Potassium acetate (83.2 mg, 848 μmol), and Potassium ferrocyanide (358 mg, 848 μmol) in 1,4-Dioxane (2.5 mL) and H₂O (2.5 mL) was sparged with N₂ atmosphere for 5 min, and then tBuXPhos (18 mg, 42.4 μmol) and tBuXPhos Pd G3 (33.7 mg, 42.4 mol) was added. The vial was sealed with a rubber septum. The mixture was stirred at 100° C. for 16 h. LCMS showed 15% desired MS. 50 mL H₂O was added, and the mixture was extracted with DCM (2×20 mL). The combined organic layers were washed with brine (20 mL), dried over anhydrous Na₂SO₄, filtered and concentrated to dryness in vacuo to give a yellow solid (500 mg). The yellow solid was subjected to column chromatography over silica gel (gradient elution: 0-9% EtOAc in petroleum ether). The desired fractions were collected, and concentrated to dryness in vacuo to give a white solid (237 mg, 420 μmol, 49.5% yield, 82.7% purity).

TLC: petroleum ether/ethyl acetate=5:1, R_{f}=0.3

LC-MS (ESI⁺) m/z: 467.3 (M+H)⁺.

Step 2: A 40 mL thread vial was equipped with magnetic stirrer. (5R, 6S)-5-(4-(4-(dimethoxymethyl)piperidin-1-yl)phenyl)-6-phenyl-5,6,7,8-tetrahydronaphthalene-2-carbonitrile (220 mg, 390 μmol) was added into HCl/dioxane (3 mL) at 20° C. The vial was scaled with a rubber septum. The reaction was stirred at 50° C. for 16 h. LCMS showed 55% desired MS. The reaction was concentrated to dryness in vacuo to give (5R, 6S)-5-(4-(4-formylpiperidin-1-yl)phenyl)-6-phenyl-5,6,7,8-tetrahydronaphthalene-2-carbonitrile (280 mg, 368 μmol, 94.3% yield, 55.3% purity) as a yellow solid.

LC-MS (ESI⁺) m/z: 421.0 (M+H)⁺.

Step 3: A solution of (S)-3-(1-oxo-5-(piperazin-1-yl)isoindolin-2-yl)piperidine-2,6-dione, TsOH salt (302 mg, 358 μmol), (5R,6S)-5-(4-(4-formylpiperidin-1-yl)phenyl)-6-phenyl-5,6,7,8-tetrahydronaphthalene-2-carbonitrile (140 mg, 239 μmol) and Sodium acetate (97.9 mg, 1.19 mmol) in DCM (2 mL) and MeOH (2 mL) was stirred at 50° C. for 3 h, Then acetic acid (28.7 mg, 477 μmol) and sodium triacetoxyborohydride (101 mg, 477 μmol) was added. The mixture was stirred at 25° C. for 16 h. The mixture was treated with H₂O (20 mL), extracted with EtOAc (20 mL*2). The combined extracts was dried over anhydrous Na₂SO₄, filtered and concentrated to dryness in vacuo to give a residue. The residue was purified by prep-HPLC (column: Phenomenex C18 80*30 mm*3 um; mobile phase: [water(FA)-ACN]; B % 11%-51%, 8 min) to give (5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalene-2-carbonitrile (20.4 mg, 11.6% yield). LC-MS (ESI⁺) m/z: 733.4 (M+H)⁺.

LCMS: calc. for C₄₆H₄₈N₆O₃: 732.38, found: [M+H]⁺ 733.4.

HPLC: 99.415% purity at 220 nm.

¹HNMR (400 MHZ, DMSO-d₆) δ=10.95 (s, 1H), 8.16 (s, 1H), 7.75 (s, 1H), 7.55-7.47 (m, 2H), 7.20-7.13 (m, 3H), 7.09-7.03 (m, 3H), 6.87 (br d, J=6.4 Hz, 2H), 6.56 (d, J=8.8 Hz, 2H), 6.20 (d. J=8.6 Hz, 2H), 5.05 (dd, J=5.0, 13.1 Hz, 1H), 4.37-4.30 (m, 2H), 4.24-4.17 (m, 1H), 3.53 (br d, J=12.4 Hz, 2H), 3.28 (br d, J=3.6 Hz, 6H), 3.18 (br dd, J=5.7, 18.2 Hz, 2H), 3.09-2.97 (m, 2H), 2.96-2.84 (m, 2H), 2.60 (br s, 4H), 2.43-2.37 (m, 1H), 2.23-2.12 (m, 3H), 2.00-1.92 (m, 1H), 1.84-1.72 (m, 3H), 1.64 (br s, 1H), 1.20-1.10 (m, 2H) SFC: retention time, 4.051 min; Area, 83.101%;

### Example 244. Preparation of (I-104) (S)-3-(5-(4-((1-(4-((1R,2S)-6-amino-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

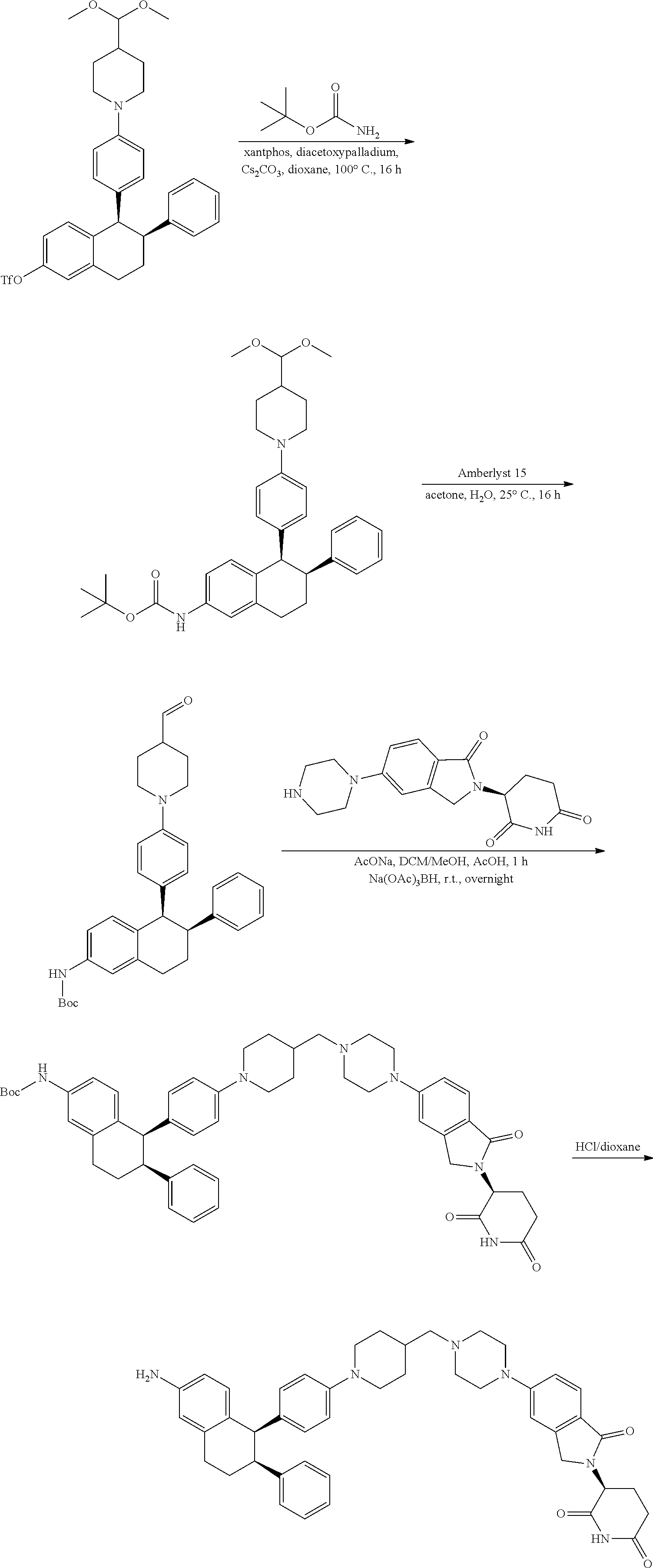

Step 1: (5R,6S)-5-(4-(4-(dimethoxymethyl)piperidin-1-yl)phenyl)-6-phenyl-5,6,7,8-tetrahydronaphthalen-2-yl trifluoromethanesulfonate (480 mg, 1 Eq, 814 μmol) and 4,5-Bis(diphenylphosphino)-9,9-dimethyl xanthene (94.2 mg, 0.2 Eq, 163 μmol) were combined in a nitrogen-purged flask. Diacetoxypalladium (18.3 mg, 0.1 Eq, 81.4 μmol), tert-butyl carbamate (143 mg, 1.5 Eq, 1.22 mmol) and Cesium carbonate (530 mg, 130 μL, 2 Eq, 1.63 mmol) were added to the reaction flask in 1,4-dioxane (2 mL) under N₂ atmosphere. The reaction mixture was stirred under dry nitrogen at 100° C. for 16 hour. TLC (petroleum ether:ethyl acetate=5:1, UV, R_{f}=0.4) showed one main new spot was observed. LCMS showed 48.86% desired MS. The reaction was quenched with water (100 mL) and extracted with ethyl acetate (200 mL*2). The organic layer was washed with brine (50 mL) and dried over Na₂SO₄ and concentrated in vacuo to give the residue as a black oil. The black oil was subjected to column chromatography over silica gel (gradient elution: 0-20% EtOAc). LCMS showed 100% desired MS. The desired fractions were collected, and concentrated to dryness in vacuo to give tert-butyl ((5R,6S)-5-(4-(4-(dimethoxymethyl)piperidin-1-yl)phenyl)-6-phenyl-5,6,7,8-tetrahydronaphthalen-2-yl)carbamate (198 mg, 356 μmol, 43.7%, 100% Purity) as a colorless oil.

LC-MS (ESI⁺) m/z: 557.4 (M+H)⁺.

Step 2: To a solution of tert-butyl ((5R,6S)-5-(4-(4-(dimethoxymethyl)piperidin-1-yl)phenyl)-6-phenyl-5,6,7,8-tetrahydronaphthalen-2-yl)carbamate (100 mg, 1 Eq, 180 μmol) in acetone (5 mL) was added 42 μL of H₂O (5 μL) and amberlyst15ion-exchangeresin (141 mg, 2.5 Eq, 449 μmol). The reaction mixture was stirred at 25° C. for 16 hour. LCMS showed 90.95% desired MS. The reaction solution filter and spin dry to give tert-butyl ((5R,6S)-5-(4-(4-formylpiperidin-1-yl)phenyl)-6-phenyl-5,6,7,8-tetrahydronaphthalen-2-yl)carbamate (50 mg, 89 μmol, 50%), 90.95% purity) as a colorless solid. LC-MS (ESI⁺) m/z: 529.2 (M+H)⁺.

Step 3: A mixture of tert-butyl ((5R,6S)-5-(4-(4-formylpiperidin-1-yl)phenyl)-6-phenyl-5,6,7,8-tetrahydronaphthalen-2-yl)carbamate (30 mg, 1 Eq, 59 μmol), (R)-3-(1-oxo-5-(piperazin-1-yl)isoindolin-2-yl)piperidine-2,6-dione, Tosylic acid (29 mg, 1 Eq, 59 μmol) and Sodium acetate (24 mg, 5 Eq, 0.29 mmol) in DCM (1.5 mL) and MeOH (1.5 mL) at 20° C. for 0.5 hour, then acetic acid (11 mg, 10 μL, 3 Eq, 0.18 mmol) at 20° C. for 1 hour, then added Na(OAc)₃BH (25 mg, 2 Eq, 0.12 mmol) to the mixture, and the mixture was stirred at 25° C. for 16 hour. LCMS showed 70.47% desired MS. The reaction was quenched with water (50 mL) and extracted with ethyl acetate (50 mL*2). The organic layer was washed with brine (50 mL) and dried over Na₂SO₄ and concentrated in vacuo to give tert-butyl ((5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalen-2-yl)carbamate (30 mg, 36 μmol, 62%) as a brown oil. LC-MS (ESI⁺) m/z: 823.5 (M+H)⁺

Step 4: A 50 mL thread vial equipped with magnetic stirrer. A solution of tert-butyl ((5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalen-2-yl)carbamate (30 mg, 1 Eq, 36 μmol) in HCL/dioxane (5 mL) was stirred at 25° C. for 16 hour. LCMS showed 36.78% desired MS. The reaction was concentrated and purified by preparative high-performance liquid chromatography. Condition: water(HCl)-CAN; Column: Phenomenex C18 150*40 mm*5 um; A: water(HCl); B: CAN; At the beginning: A (99%) and B (1%); At the end: A: (70%) and B (30%); Gradient Time (min) 10; 100% B hold Time (min) 2, Flow Rate (ml/min) 60. The pure fractions were collected and the solvent was evaporated under vacuum. The aqueous layer was lyophilized to dryness to give (S)-3-(5-(4-((1-(4-((1R,2S)-6-amino-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (7 mg, 9 μmol, 30%, 95.05% Purity) as a white solid. LC-MS (ESI+) m/z: 723.2 (M+H)+.

LCMS: calc. for C₄₅H₅₀N₆O₃: 722.39, found: [M+H]⁺ 723.2.

HPLC: 95.056% purity at 220 nm.

¹H NMR (400 MHZ, DMSO-d₆) δ=11.00-10.95 (m, 1H), 7.62-7.58 (m, 1H), 7.23-7.12 (m, 8H), 7.03-6.99 (m, 1H), 6.97-6.94 (m, 1H), 6.87-6.85 (m, 2H), 6.40-6.22 (m, 2H), 5.10-5.03 (m, 1H), 4.39-4.32 (m, 2H), 4.27-4.21 (m, 1H), 4.02-3.94 (m, 2H), 3.67-3.50 (m, 7H), 3.20-3.03 (m, 9H), 2.94-2.85 (m, 1H), 2.70-2.65 (m, 1H), 2.34-2.32 (m, 1H), 2.19-2.07 (m, 2H), 2.02-1.89 (m, 4H), 1.82-1.77 (m, 1H).

SFC: retention time, 4.276 min; Area, 87.795%;

### Example 245. Preparation of (I-46) (S)-3-(1-oxo-5-(4-((1-(4-((1R,2S)-2-phenyl-6-(trifluoromethyl)-1,2,3,4-tetrahydronaphthalen-1-yl)phenyl)piperidin-4-yl)methyl)piperazin-1-yl)isoindolin-2-yl)piperidine-2,6-dione

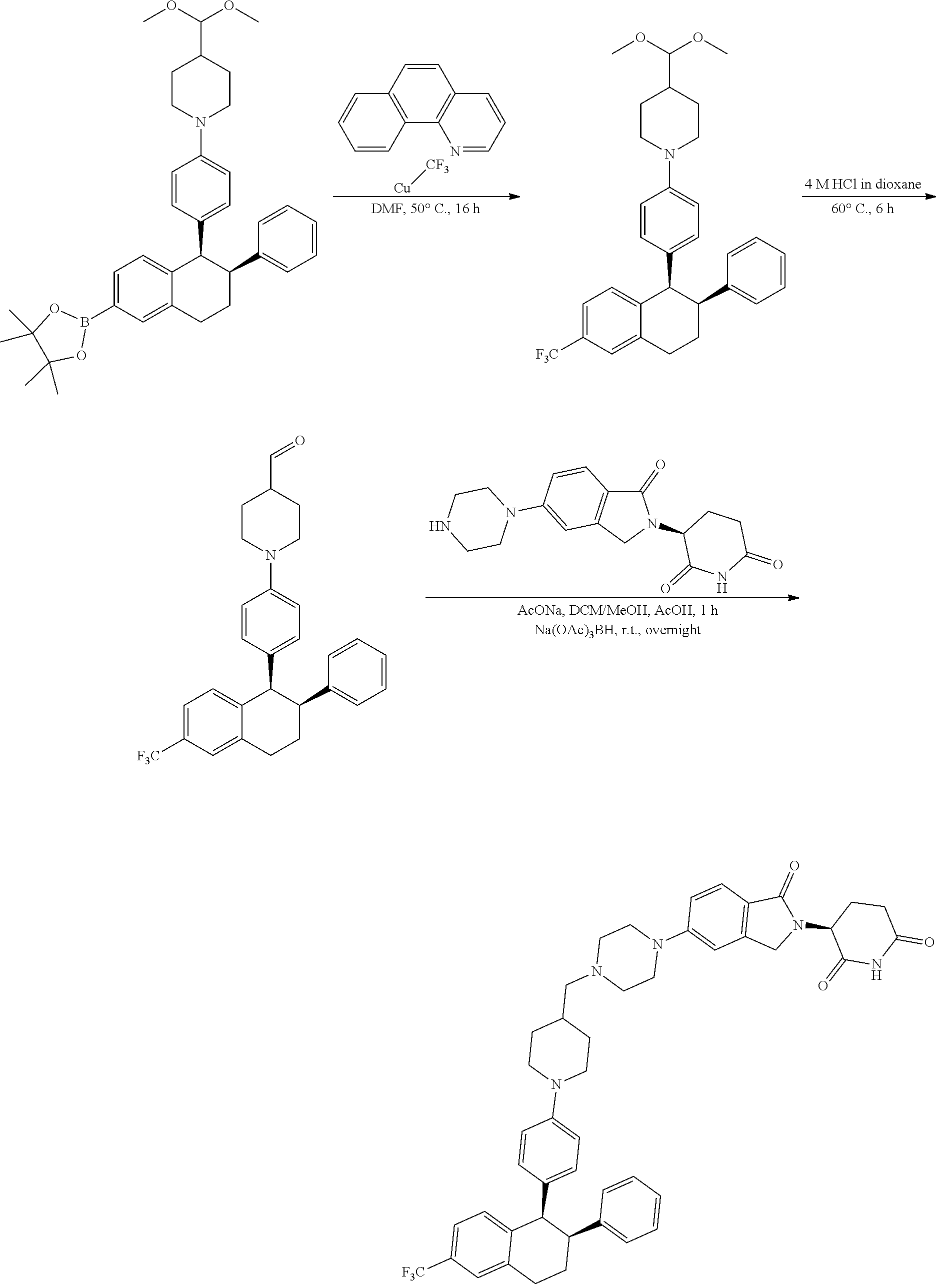

Step 1: A solution of 4-(dimethoxymethyl)-1-(4-((1R,2S)-2-phenyl-6-(4,4,5,5-tetramethyl-1,3,2-dioxaborolan-2-yl)-1,2,3,4-tetrahydronaphthalen-1-yl)phenyl)piperidine (150 mg, 1 Eq, 264 μmol) and Reactant II (124 mg, 1.5 Eq, 396 μmol) in DMF (4 mL) was stirred at 50° C. for 16 hour, The mixture was stirred at 50° C. for 16 h. LCMS showed 36.093% desired MS. TLC (petroleum ether:ethyl acetate=5:1, UV) showed one main new spot was observed. The reaction was purified by flash column (petroleum ether/ethyl acetate from 100/0 to 0/100) and the organic layer was concentrated in vacuo to give 4-(dimethoxymethyl)-1-(4-((1R,2S)-2-phenyl-6-(trifluoromethyl)-1,2,3,4-tetrahydronaphthalen-1-yl)phenyl)piperidine (110 mg, 216 μmol, 81.7% yield, 100% purity) as a yellow solid.

LC-MS (ESI+) m/z: 510.1 (M+H)⁺

Step 2: To a solution of (4-(dimethoxymethyl)-1-(4-((1R,2S)-2-phenyl-6-(trifluoromethyl)-1,2,3,4-tetrahydronaphthalen-1-yl)phenyl)piperidine (110 mg, 1 Eq, 216 μmol) in 4 M HCl in dioxane (4 mL). The mixture was stirred at 60° C. for 6 hour. LCMS showed 100% desired MS. The reaction was concentrated to give 1-(4-((1R,2S)-2-phenyl-6-(trifluoromethyl)-1,2,3,4-tetrahydronaphthalen-1-yl)phenyl)piperidine-4-carbaldehyde (100 mg, 216 μmol, 99.9% yield, 100% purity) as a white yellow. LC-MS (ESI+) m/z: 464.2 (M+H)⁺

Step 3: A solution of 1-(4-((1R,2S)-2-phenyl-6-(trifluoromethyl)-1,2,3,4-tetrahydronaphthalen-1-yl)phenyl)piperidine-4-carbaldehyde (100 mg, 1 Eq, 216 μmol) and (S)-3-(1-oxo-5-(piperazin-1-yl)isoindolin-2-yl)piperidine-2,6-dione, 3Tosylic acid (180 mg, 0.987 Eq, 213 μmol) and AcONa (108 mg, 5 Eq, 1.08 mmol) in DCM (2 mL) and MeOH (2 mL) was stirred at 20° C. for 30 mins, Then NaBH(OAc); (40.7 mg, 3 Eq, 647 μmol) and Acetic acid (51.8 mg, 49.6 uL, 4 Eq, 863 μmol) was added. The mixture was stirred at 20° C. for 16 h. LCMS showed 89.038% desired MS. The reaction was concentrated to give a brown solid. The brown solid was purified by preparative high-performance liquid chromatography. Condition: Column: 2 Phenomenex Gemini C18 75*40 mm*3 um A: water(FA); B: CAN; at the beginning: A (65%) and B (35%); at the end: A: (35%) and B (65%); Gradient Time (min) 7; 100% B hold Time (min) 2, Flow Rate (ml/min) 25. The pure fractions were collected and the solvent was evaporated under vacuum. The aqueous layer was lyophilized to dryness to give (S)-3-(1-oxo-5-(4-((1-(4-((1R,2S)-2-phenyl-6-(trifluoromethyl)-1,2,3,4-tetrahydronaphthalen-1-yl)phenyl)piperidin-4-yl)methyl)piperazin-1-yl)isoindolin-2-yl)piperidine-2,6-dione (40.8 mg, 52.6 μmol, 24.4% yield, 100% purity) as a white solid. LC-MS (ESI⁺) m/z: 708.4 (M+H)⁺

LCMS: calc. for C₄₆H₄₈F₃N₅O₃, found: [M+H]⁺ 776.3.

HPLC: 96.737% purity at 220 nm.

¹H NMR (400 MHZ, DMSO-d₆) δ=10.95 (s, 1H), 8.21 (s, 1H), 7.62 (s, 1H), 7.52 (d, J=8.5 Hz, 1H), 7.40 (br d, J=7.8 Hz, 1H), 7.20-7.04 (m, 6H), 6.87 (br d, J=6.5 Hz, 2H), 6.57 (d, J=8.8 Hz, 2H), 6.22 (d, J=8.8 Hz, 2H), 5.05 (dd, J=5.0, 13.3 Hz, 1H), 4.33 (br d, J=16.6 Hz, 2H), 4.24-4.16 (m, 1H), 3.53 (br d, J=10.0 Hz, 2H), 3.35 (br d, J=10.5 Hz, 6H), 3.20 (br d, J=5.3 Hz, 1H), 3.12-3.00 (m, 1H), 2.96-2.84 (m, 1H), 2.63-2.52 (m, 6H), 2.40-2.32 (m, 1H), 2.19 (br d, J=7.0 Hz, 3H), 2.00-1.93 (m, 1H), 1.85-1.70 (m, 3H), 1.64 (br s, 1H), 1.40-1.03 (m, 3H)

SFC: retention time, 2.612; Area, 91.907%;

### Example 246. Preparation of (I-45) ((5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalen-2-yl)boronic acid

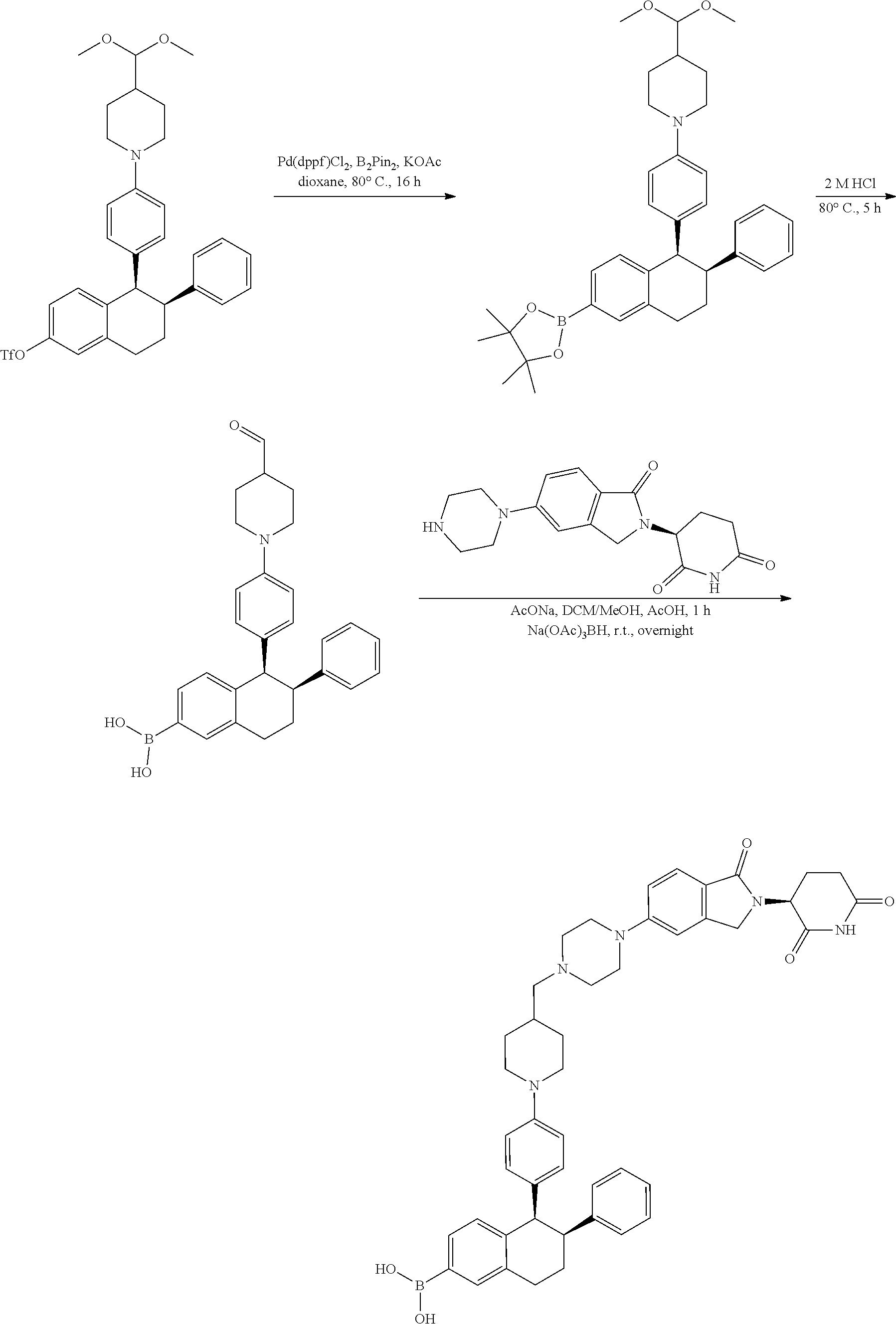 [SMILES: COC(OC)C1CCN(c2ccc([C@@H]3c4ccc(OS(=O)(=O)C(F)(F)F)cc4CC[C@@H]3c3ccccc3)cc2)CC1 ; COC(OC)C1CCN(c2ccc([C@@H]3c4ccc(B5OC(C)(C)C(C)(C)O5)cc4CC[C@@H]3c3ccccc3)cc2)CC1 ; O=CC1CCN(c2ccc([C@@H]3c4ccc(B(O)O)cc4CC[C@@H]3c3ccccc3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(B(O)O)cc8CC[C@@H]7c7ccccc7)cc6)CC5)CC4)ccc3C2=O)C(=O)N1]

Step 1: (5R,6S)-5-(4-(4-(dimethoxymethyl)piperidin-1-yl)phenyl)-6-phenyl-5,6,7,8-tetrahydronaphthalen-2-yl trifluoromethanesulfonate (400 mg, 1 Eq, 678 μmol), 4,4,4′,4′,5,5,5′,5′-octamethyl-2,2′-bi (1,3,2-dioxaborolane) (258 mg, 1.5 Eq, 1.02 mmol), potassium acetate (133 mg, 2 Eq, 1.36 mmol) and 1,4-Dioxane (5 mL) were added to a 100 mL round-bottomed flask. The resulting mixture was sparged with N₂ for 5 min, followed by Pd(dppf)Cl₂ (55.4 mg, 0.1 Eq, 67.8 μmol) were added. The resultant mixture was sparged with N₂ for 5 min and stirred under argon for 12 h at 90° C. After this time, the mixture was cooled to room temperature. LCMS showed 61.35% desired MS. The mixture was discarded without purification. TLC (petroleum ether:ethyl acetate=5:1, UV) showed one main new spot was observed. 50 mL water was added, and the mixture was extracted with dichloromethane (30 mL×2). The combined organic layers were washed with brine (20 mL), dried over anhydrous Na₂SO₄, filtered and concentrated to dryness in vacuo to give a yellow oil. The yellow oil was purified by flash column (petroleum ether/ethyl acetate from 100/0 to 0/100) and the organic layer was concentrated in vacuo to give 4-(dimethoxymethyl)-1-(4-((1R,2S)-2-phenyl-6-(4,4,5,5-tetramethyl-1,3,2-dioxaborolan-2-yl)-1,2,3,4-tetrahydronaphthalen-1-yl)phenyl)piperidine (270 mg, 476 μmol, 70.1% yield) as a yellow solid.

LC-MS (ESI+) m/z: 568.3 (M+H)⁺

Step 2: To a solution of (4-(dimethoxymethyl)-1-(4-((1R,2S)-2-phenyl-6-(4,4,5,5-tetramethyl-1,3,2-dioxaborolan-2-yl)-1,2,3,4-tetrahydronaphthalen-1-yl)phenyl)piperidine (100 mg, 1 Eq, 176 μmol) in H₂O (0.75 mL) was added 2 M hydrogen chloride (5 mL). The mixture was stirred at 80° C. for 16 hr. LCMS showed 69.231% desired MS. The reaction was concentrated in vacuo to give ((5R, 6S)-5-(4-(4-formylpiperidin-1-yl)phenyl)-6-phenyl-5,6,7,8-tetrahydronaphthalen-2-yl)boronic acid (100 mg, 158 μmol, 89.4%, 69.2% purity) as a yellow solid. LC-MS (ESI+) m/z: 442.3 (M+H)⁺

Step 3: A solution of AcONa (68 mg, 5 Eq, 0.68 mmol) and (S)-3-(1-oxo-5-(piperazin-1-yl)isoindolin-2-yl)piperidine-2,6-dione, TsOH acid (100 mg, 0.87 Eq, 118 μmol) and ((5R,6S)-5-(4-(4-formylpiperidin-1-yl)phenyl)-6-phenyl-5,6,7,8-tetrahydronaphthalen-2-yl)boronic acid (60 mg, 1 Eq, 0.14 mmol) in DCM (2 mL) and MeOH (2 mL) was stirred at 20° C. for 30 mins, Then NaBH(OAc) 3 (26 mg, 3 Eq, 0.41 mmol) and Acetic acid (33 mg, 31 μL, 4 Eq, 0.55 mmol) was added. The mixture was stirred at 20° C. for 16 h. LCMS showed 40.930% desired MS. The reaction was concentrated to give a brown solid. The brown solid was purified by preparative high-performance liquid chromatography. Condition: Column: Boston Green ODS 150*30 mm*5 um A: water(FA) B: CAN; at the beginning: A (80%) and B (20%); at the end: A: (50%) and B (50%); Gradient Time (min) 6; 100% B hold Time (min) 1, Flow Rate (ml/min) 30. The pure fractions were collected and the solvent was evaporated under vacuum. The aqueous layer was lyophilized to dryness to give ((5R,6S)-5-(4-(4-((4-(2-((S)-2,6-dioxopiperidin-3-yl)-1-oxoisoindolin-5-yl)piperazin-1-yl)methyl)piperidin-1-yl)phenyl)-6-phenyl-5,6,7,8-tetrahydronaphthalen-2-yl)boronic acid (16.3 mg, 21.6 μmol, 16%, 99.5% purity) as a white solid. LC-MS (ESI⁺) m/z: 708.4 (M+H)⁺

LCMS: calc. for C₄₅H₅₀BN₅O₅, found: [M+H]⁺ 752.0.

HPLC: 99.5% purity at 220 nm.

¹H NMR (400 MHZ, DMSO-d₆) δ=10.95 (s, 1H), 8.14 (s, 1H), 7.93 (s, 2H), 7.67 (s, 1H), 7.53 (br d, J=7.8 Hz, 1H), 7.46 (d, J=7.8 Hz, 1H), 7.16 (br d, J=7.5 Hz, 3H), 7.11-7.05 (m, 2H), 6.84 (dd, J=7.4, 17.7 Hz, 3H), 6.56-6.53 (m, 2H), 6.21 (br d, J=8.8 Hz, 2H), 5.05 (dd, J=5.0, 13.8 Hz, 1H), 4.37-4.19 (m, 3H), 3.52 (br d, J=11.3 Hz, 2H), 3.21-2.83 (m, 7H), 2.61 (br s, 8H), 2.19 (br s, 2H), 1.97 (br d, J=7.0 Hz, 1H), 1.76 (br d, J=11.8 Hz, 3H), 1.68 (br s, 1H), 1.30-1.13 (m, 3H)

SFC: retention time, 9.777; Area, 99.213%;

### Example 247. Preparation of (I-47) (S)-3-(1-oxo-5-(4-((1-(4-((1R,2S)-2-phenyl-1,2,3,4-tetrahydronaphthalen-1-yl)phenyl)piperidin-4-yl)methyl)piperazin-1-yl)isoindolin-2-yl)piperidine-2,6-dione

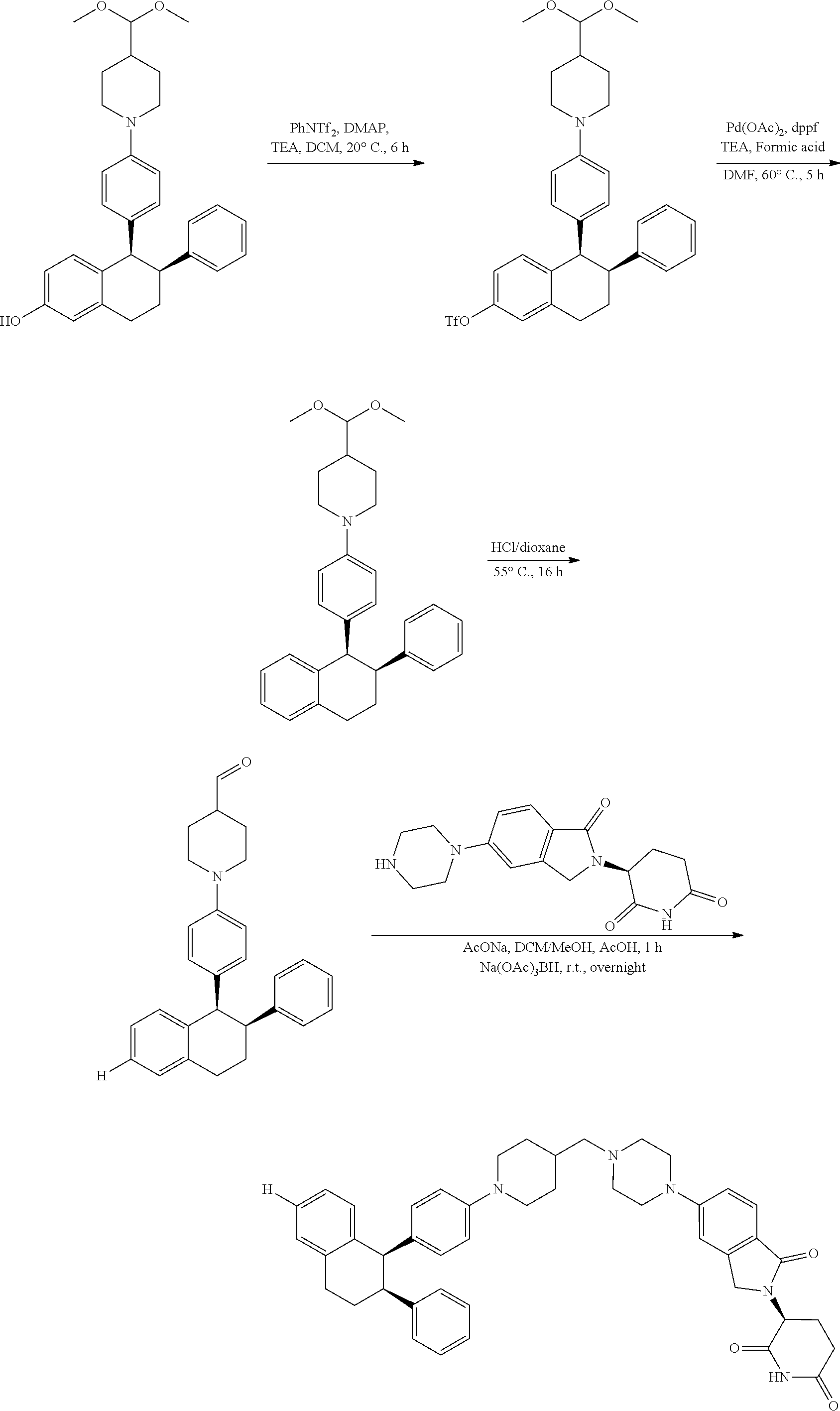 [SMILES: COC(OC)C1CCN(c2ccc([C@@H]3c4ccc(O)cc4CC[C@@H]3c3ccccc3)cc2)CC1 ; COC(OC)C1CCN(c2ccc([C@@H]3c4ccc(OS(=O)(=O)C(F)(F)F)cc4CC[C@@H]3c3ccccc3)cc2)CC1 ; COC(OC)C1CCN(c2ccc([C@@H]3c4ccccc4CC[C@@H]3c3ccccc3)cc2)CC1 ; O=CC1CCN(c2ccc([C@@H]3c4ccccc4CC[C@@H]3c3ccccc3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccccc8CC[C@@H]7c7ccccc7)cc6)CC5)CC4)ccc3C2=O)C(=O)N1]

Step 1: To a stirred solution of (5R,6S)-5-(4-(4-(dimethoxymethyl)piperidin-1-yl)phenyl)-6-phenyl-5,6,7,8-tetrahydronaphthalen-2-ol (3 g, 1 Eq. 7 mmol) and Phenyl triflimide (4 g, 1.5 Eq, 0.01 mol) in DCM (30 mL) at 20° C. was added triethylamine (0.8 g, 1 mL, 1.2 Eq, 8 mmol) and a catalytic amount of DMAP (0.2 g, 0.2 Eq, 1 mmol) was then added to the mixture and stirring was continued at 20° C. for 6 hours. LCMS showed 84.014% desired MS. TLC (petroleum ether:ethyl acetate=10:1, UV) showed one main new spot was observed. The reaction was concentrated to dryness in vacuo to give a yellow oil. The yellow oil was subjected to column chromatography over silica gel (gradient elution: 0-10% EtOAc in petroleum ether). The desired fractions were collected, and concentrated to dryness in vacuo to give (5R,6S)-5-(4-(4-(dimethoxymethyl)piperidin-1-yl)phenyl)-6-phenyl-5,6,7,8-tetrahydronaphthalen-2-yl trifluoromethanesulfonate (3.9 g, 6.6 mmol, 100%) as a yellow oil.

LC-MS (ESI+) m/z: 590.2 (M+H)⁺

Step 2: Triethylamine (257 mg, 355 μl, 5 Eq, 2.54 mmol) and Palladium diacetate (11.4 mg, 0.1 Eq, 50.9 μmol) were added to a stirred mixture of (5R,6S)-5-(4-(4-(dimethoxymethyl)piperidin-1-yl)phenyl)-6-phenyl-5,6,7,8-tetrahydronaphthalen-2-yl trifluoromethanesulfonate (300 mg, 1 Eq, 509 μmol), 1,1′-Ferrocendiylbis(diphenylphosphine) (56.4 mg, 0.2 Eq, 102 μmol) and Formaldehyde (76.4 mg, 70.1 uL, 5 Eq, 2.54 mmol)) in DMF (5 mL) at 20° C., under nitrogen. The mixture was stirred under nitrogen for 5 hours at 60° C. LCMS showed 87.974% desired MS. TLC (petroleum ether:ethyl acetate=2:1, UV) showed one main new spot was observed. 50 mL water was added, and the mixture was extracted with DCM (100 mL×2). The combined organic layers were washed with brine (10 mL), dried over anhydrous Na₂SO₄, filtered and concentrated to dryness in vacuo to give a yellow oil. The yellow oil was purified by flash column (petroleum ether/ethyl acetate from 100/0 to 0/100) and the organic layer was concentrated in vacuo to give 4-(dimethoxymethyl)-1-(4-((1R,2S)-2-phenyl-1,2,3,4-tetrahydronaphthalen-1-yl)phenyl)piperidine (200 mg, 453 μmol, 89.0%) as a yellow solid. LC-MS (ESI+) m/z: 442.3 (M+H)⁺

Step 3: To a solution of (4-(dimethoxymethyl)-1-(4-((1R,2S)-2-phenyl-1,2,3,4-tetrahydronaphthalen-1-yl)phenyl)piperidine (100 mg, 1 Eq, 226 μmol)) in HCl in dioxane (4 mL). The mixture was stirred at 55° C. for 16 hour. LCMS showed 85.493% desired MS. The reaction was concentrated to give 1-(4-((1R,2S)-2-phenyl-1,2,3,4-tetrahydronaphthalen-1-yl)phenyl)piperidine-4-carbaldehyde (85 mg, 0.18 mmol, 81%, 85.493% purity) as a white yellow. LC-MS (ESI⁺) m/z: 396.2 (M+H)⁺.

Step 4: A solution of 1-(4-((1R,2S)-2-phenyl-1,2,3,4-tetrahydronaphthalen-1-yl)phenyl)piperidine-4-carbaldehyde (100 mg, 1 Eq, 25 μmol) and (S)-3-(1-oxo-5-(piperazin-1-yl)isoindolin-2-yl)piperidine-2,6-dione, TsOH acid (140 mg, 0.655 Eq, 166 μmol) and NaOAc (126 mg, 5 Eq, 1.26 mmol) in DCM (0.5 mL) and MeOH (0.5 mL) was stirred at 20° C. for 60 mins, Then NaBH(OAc)₃ (47.7 mg, 44.7 uL, 3 Eq, 758 μmol) and Acetic acid (60.7 mg, 58.2 uL, 4 Eq, 1.01 mmol) was added. The mixture was stirred at 20° C. for 16 h. LCMS showed 80.174% desired MS. The reaction was concentrated to a brown solid. The brown solid was purified by preparative high-performance liquid chromatography. Condition: Column: Welch Xtimate C18 150*30 mm*5 um A: water(FA) B: ACN at the beginning: A (72%) and B (28%) at the end: A: (42%) and B (58%) Gradient Time (min) 7; 100% B hold Time (min) 1.7, Flow Rate (ml/min) 25. The pure fractions were collected and the solvent was evaporated under vacuum. The aqueous layer was lyophilized to dryness to give (S)-3-(1-oxo-5-(4-((1-(4-((1R,2S)-2-phenyl-1,2,3,4-tetrahydronaphthalen-1-yl)phenyl)piperidin-4-yl)methyl)piperazin-1-yl)isoindolin-2-yl)piperidine-2,6-dione (20.7 mg, 28.1 μmol, 11.1% yield, 96.1% purity) as a white solid. LC-MS (ESI⁺) m/z: 708.4 (M+H)⁺

LCMS: calc. for C₄₅H₄₉N₅O₃, found: [M+H]⁺ 708.4.

HPLC: 96.1% purity at 220 nm.

¹H NMR (400 MHZ, DMSO-d₆) δ=10.95 (s, 1H), 8.15 (s, 0.439H), 7.52 (d, J=8.5 Hz, 1H), 7.25-7.21 (m, 1H), 7.17-7.13 (m, 4H), 7.08-7.04 (m, 3H), 6.86 (d, J=6.8 Hz, 4H), 6.55 (d, J=8.8 Hz, 2H), 6.21 (d, J=8.5 Hz, 2H), 5.04 (dd, J=5.1, 13.2 Hz, 1H), 4.39-4.11 (m, 4H), 3.52 (br d, J=10.0 Hz, 2H), 3.29 (br s, 8H), 3.12-2.85 (m, 3H), 2.19 (br d, J=7.5 Hz, 3H), 1.99-1.93 (m, 1H), 1.76 (br d, J=11.3 Hz, 4H), 1.64 (br s, 1H), 1.32-0.97 (m, 3H)

SFC: retention time, 3.274; Area, 93.534%;

### Example 248. Preparation of (I-299) 1-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)dihydropyrimidine-2,4 (1H,3H)-dione

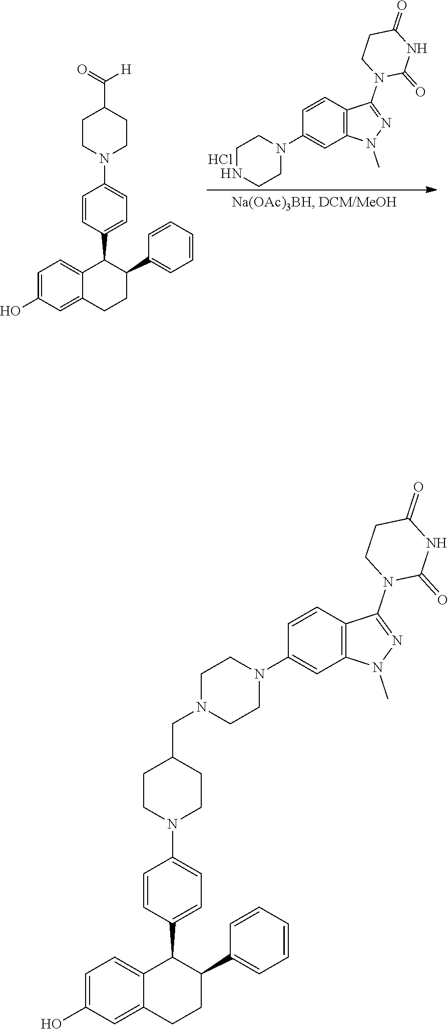 [SMILES: O=CC1CCN(c2ccc([C@@H]3c4ccc(O)cc4CC[C@@H]3c3ccccc3)cc2)CC1 ; Cl ; Cn1nc(N2CCC(=O)NC2=O)c2ccc(N3CCNCC3)cc21 ; Cn1nc(N2CCC(=O)NC2=O)c2ccc(N3CCN(CC4CCN(c5ccc([C@@H]6c7ccc(O)cc7CC[C@@H]6c6ccccc6)cc5)CC4)CC3)cc21]

Step 1: To a solution of 1-(1-methyl-6-(piperazin-1-yl)-1H-indazol-3-yl)dihydropyrimidine-2,4 (1H,3H)-dione hydrochloride (20 mg, 54.82 μmol, 1 eq.) in DCE (2 mL) and MeOH (3 mL) was added 1-(4-((1R,2S)-6-hydroxy-2-phenyl-1,2,3,4-tetrahydronaphthalen-1-yl)phenyl)piperidine-4-carbaldehyde (22.56 mg, 54.82 μmol, 1 eq.) and NaBH(OAc) 3 (58.09 mg, 274.10 μmol, 5 eq.). The reaction was stirred at 20° C. for 1 hr. LCMS showed the desired ms was found. The mixture was purified by Prep-HPLC (Phenomenex Gemini NX 150*30 mm*5 um; Condition: water (NH₄HCO₃)-ACN; Begin B 56, End B 86, Gradient Time (min) 11, 100% B Hold Time (min) 2, FlowRate (ml/min) 25, Injections 1, HPLC 95) to give 1-(6-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-methyl-1H-indazol-3-yl)dihydropyrimidine-2,4 (1H,3H)-dione (11 mg, 14.84 μmol, 27.07% yield, 97.66% purity) as a white solid. LC-MS (ESI⁺) m/z: 724.4 (M+H)⁺.

### Example 249. Preparation of (I-191) 3-(4-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione

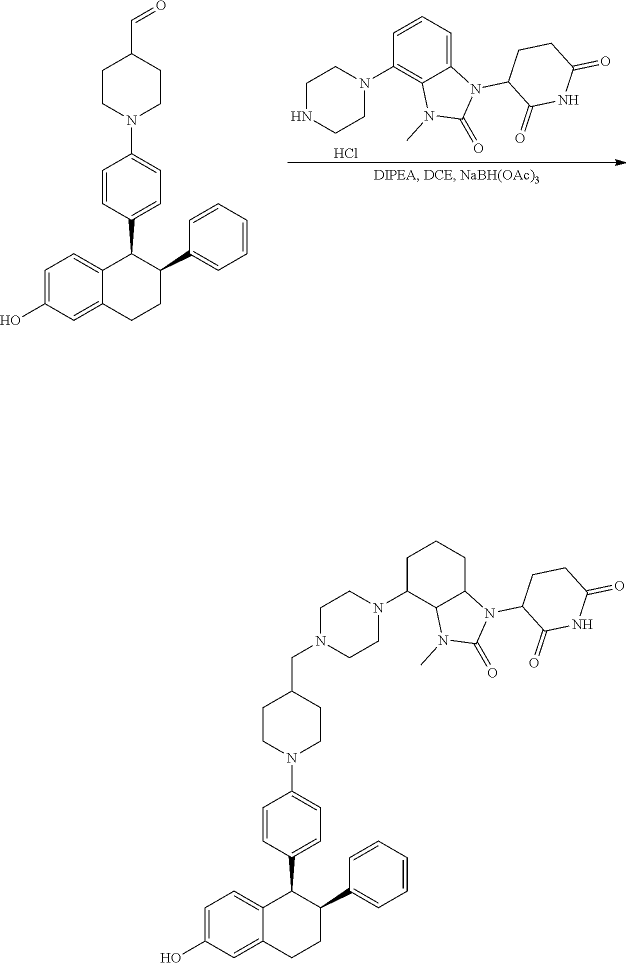 [SMILES: O=CC1CCN(c2ccc([C@@H]3c4ccc(O)cc4CC[C@@H]3c3ccccc3)cc2)CC1 ; Cn1c(=O)n(C2CCC(=O)NC2=O)c2cccc(N3CCNCC3)c21 ; Cl ; CN1C(=O)N(C2CCC(=O)NC2=O)C2CCCC(N3CCN(CC4CCN(c5ccc([C@@H]6c7ccc(O)cc7CC[C@@H]6c6ccccc6)cc5)CC4)CC3)C21]

Step 1: To a solution of 1-(4-((1R,2S)-6-hydroxy-2-phenyl-1,2,3,4-tetrahydronaphthalen-1-yl)phenyl)piperidine-4-carbaldehyde (50 mg, 121.50 μmol, 1 eq.) and 3-(3-methyl-2-oxo-4-(piperazin-1-yl)-2,3-dihydro-1H-benzo[d]imidazol-1-yl)piperidine-2,6-dione hydrochloride (50.06 mg, 145.80 μmol, 1.2 eq.) in DCE (3 mL) was added sodium;triacetoxyboranuide (77.25 mg, 364.49 μmol, 3 eq.). The mixture was stirred at 25° C. for 2 hr. LC-MS showed one main peak with desired mass was detected. The reaction mixture or solution was concentrated to dryness. The residue was purified by prep-HPLC(Column Boston Green ODS 150*30 mm*5 um, Condition water(HCl)-CAN, Begin B 20, End B 40, Gradient Time (min) 10.5, 100% B Hold Time (min) 1.5, FlowRate (ml/min) 25, Injections 4, HPLC 96). 3-(4-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione (13 mg, 17.59 μmol, 14.48% yield) was obtained as a white solid.

LC-MS (ESI⁺) m/z: 739.6 (M+H)⁺.

LCMS: calc. for C₄₅H₅₀N₆O₄:738.39, found: [M+H]⁺ 739.6.

HPLC: 100% purity at 220 nm.

¹H NMR: ¹H NMR (400 MHZ, METHANOL-d₄) 6 ppm 7.07-7.28 (m, 7H) 7.01 (br d, J=8.46 Hz, 1H) 6.81-6.87 (m, 2H) 6.56-6.71 (m, 4H) 6.50-6.55 (m, 1H) 5.30 (s, 1H) 4.30-4.42 (m, 1H) 3.52-3.82 (m, 8H) 3.33-3.50 (m, 9H) 3.25-3.29 (m, 1H) 3.00-3.14 (m, 2H) 2.76-2.98 (m, 3H) 2.28-2.43 (m, 1H) 2.10-2.25 (m, 4H) 1.72-1.93 (m, 3H)

EXAMPLE 250, Preparation of (I-190) 3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione

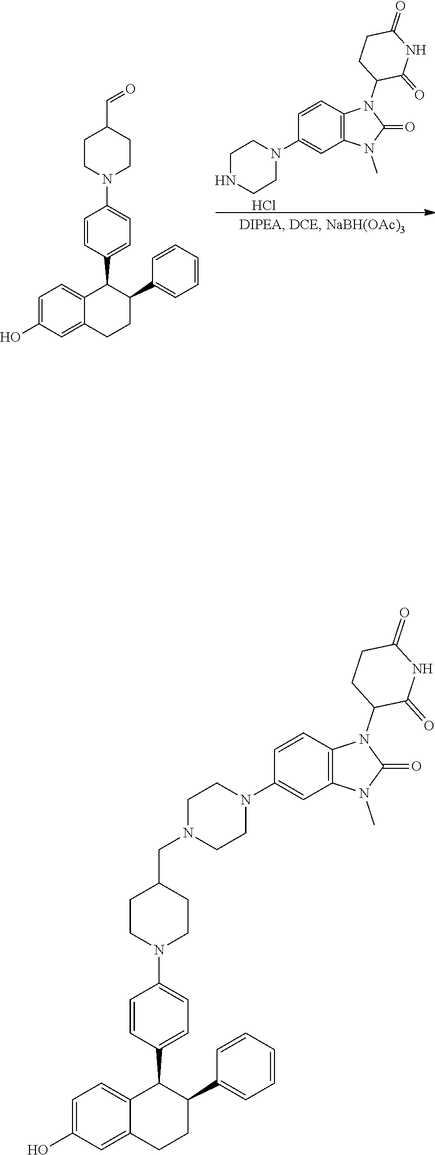 [SMILES: O=CC1CCN(c2ccc([C@@H]3c4ccc(O)cc4CC[C@@H]3c3ccccc3)cc2)CC1 ; Cn1c(=O)n(C2CCC(=O)NC2=O)c2ccc(N3CCNCC3)cc21 ; Cl ; Cn1c(=O)n(C2CCC(=O)NC2=O)c2ccc(N3CCN(CC4CCN(c5ccc([C@@H]6c7ccc(O)cc7CC[C@@H]6c6ccccc6)cc5)CC4)CC3)cc21]

Step 1: To a solution of 1-(4-((1R,2S)-6-hydroxy-2-phenyl-1,2,3,4-tetrahydronaphthalen-1-yl)phenyl)piperidine-4-carbaldehyde (50 mg, 121.50 μmol, 1 eq.) and 3-(3-methyl-2-oxo-5-(piperazin-1-yl)-2,3-dihydro-1H-benzo[d]imidazol-1-yl)piperidine-2,6-dione hydrochloride (50.06 mg, 145.80 μmol, 1.2 eq.) in DCE (3 mL) was added sodium;triacetoxyboranuide (77.25 mg, 364.49 μmol, 3 eq.). The mixture was stirred at 25° C. for 2 hr. LC-MS showed one main peak with desired mass was detected. The reaction mixture or solution was concentrated to dryness. The residue was purified by prep-HPLC(Column Boston Green ODS 150*30 mm*5 um, Condition water(HCl)-CAN, Begin B 20, End B 40, Gradient Time (min) 10.5, 100% B Hold Time (min) 1.5, FlowRate (ml/min) 25, Injections 4, HPLC 96). 3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione (14.5 mg, 19.62 μmol, 16.15% yield) was obtained as a white solid. LC-MS (ESI⁺) m/z: 739.6 (M+H)⁺. LCMS: calc. for C₄₅H₅₀N₆O₄:738.39, found: [M+H]⁺ 739.6.

HPLC: 99.45% purity at 220 nm.

¹H NMR: ¹H NMR (400 MHZ, METHANOL-d₄) δ ppm 7.32 (d, J=8.70 Hz, 2H) 7.11-7.17 (m, 3H) 7.02-7.07 (m, 1H) 6.93-6.98 (m, 1H) 6.80-6.88 (m, 3H) 6.64-6.71 (m, 4H) 6.48-6.58 (m, 1H) 5.31 (dd, J=12.70, 5.30 Hz, 1H) 4.41 (d, J=5.36 Hz, 1H) 3.61-3.82 (m, 7H) 3.41 (s, 5H) 3.20-3.29 (m, 3H) 3.01-3.14 (m, 2H) 2.87-2.97 (m, 1H) 2.69-2.85 (m, 2H) 2.37-2.54 (m, 1H) 2.11-2.30 (m, 4H) 1.80-1.99 (m, 3H)

### Example 251. Preparation of (I-419) (S)-3-(5-(4-((1-(4-((1R,2S,4R)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione, (I-332) (S)-3-(5-(4-((1-(4-((1R,2S,4S)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione, (I-420) (S)-3-(5-(4-((1-(4-((1S,2R,4R)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione, and (I-331) (S)-3-(5-(4-((1-(4-((1S,2R,4S)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione

[chemical structure]

Step 1: To a solution of methyl 4-oxopentanoate (23.61 g, 126.79 mmol, 1.1 eq.) in MeOH (100 mL) was added 4-methylbenzenesulfonohydrazide (15 g, 115.26 mmol, 14.27 mL, 1 eq.). The mixture was stirred at 25° C. for 24 hour. LCMS showed the desired MS was detected. The mixture was filtered and solid was collected, washed with MeOH (10 mL×2), treated under vacuum to yield methyl (E)-4-(2-tosylhydrazineylidene)pentanoate (30 g, 98% purity, 87.24% yield) as a white solid.

LC-MS (ESI⁺) m/z: 298.8 (M+H)⁺

Step 2: A mixture of (3-methoxyphenyl)boronic acid (10 g, 65.81 mmol, 1 eq.), methyl (E)-4-(2-tosylhydrazineylidene)pentanoate (21.60 g, 72.39 mmol, 1.1 eq.) and K₂CO₃ (22.74 g, 164.52 mmol, 2.5 eq.) in dioxane (150 mL) was degassed and purged with N₂ for 3 times, and then the mixture was stirred at 100° C. for 16 hour under N₂ atmosphere. TLC (PE/EtOAc=10:1) showed new spot was observed. The reaction mixture was concentrated under reduced pressure to remove dioxane to get the crude product, which was purified by silica gel column chromatography (from PE/EtOAc=10/1 to 5/1, TLC: PE/EtOAc=10/1, R_{f}=0.63) to yield desired methyl 4-(3-methoxyphenyl)pentanoate (12 g, 82.03% yield) as colorless liquid.

Step 3: To a solution of methyl 4-(3-methoxyphenyl)pentanoate (10 g, 44.99 mmol, 0.1 mL, 1 eq.) was added TfOH (169.60 g, 1.13 mol, 100.00 mL, 25.12 eq.). The mixture was stirred at 20° C. for 1 hour. LCMS showed the desired MS was detected. The mixture was adjusted to pH 5-6 with NaHCO₃ (10%, 50 mL), which was extracted with EA (50 mL×3). The combined organic layers were dried over Na₂SO₄, filtered and concentrated to afford the crude product. The crude product was purified by silica gel chromatography (from PE/EtOAc=3/1 to 2/1, TLC: PE/EtOAc=3/1, R_{f}=0.60) to give the 6-methoxy-4-methyl-3,4-dihydronaphthalen-1 (2H)-one (2.7 g, 90% purity, 31.55% yield) as a white solid.

LC-MS (ESI⁺) m/z: 191.2 (M+H)⁺.

Step 4: To a solution of 1-(4-bromophenyl)-4-(dimethoxymethyl)piperidine (5.45 g, 17.35 mmol, 1.1 eq.) in THF (50 mL) was added dropwise n-BuLi (2.5 M, 9.46 mL, 1.5 eq.) at −50° C. After addition, the mixture was stirred at this temperature for 0.5 hour, and then 6-methoxy-4-methyl-3,4-dihydronaphthalen-1 (2H)-one (3 g, 15.77 mmol, 1 eq.) was added dropwise at −40° C. The resulting mixture was stirred at 0° C. for 2 hour. LCMS showed desired compound was detected. The mixture was quenched by addition of 10 mL of water, and then the mixture was extracted with EA (50 mL×3). The combined organic layer was dried over Na₂SO₄, filtered and concentrated to afford the crude product, which was purified on silica gel column chromatography (from PE/EtOAc=3/1 to 2/1, TLC: PE/EtOAc=3/1, R_{f}=0.49) to yield 1-(4-(4-(dimethoxymethyl)piperidin-1-yl)phenyl)-6-methoxy-4-methyl-1,2,3,4-tetrahydronaphthalen-1-ol (3 g, 95% purity, 53.64% yield) as a yellow solid.

LC-MS (ESI⁺) m/z: 426.3 (M+H)⁺.

Step 5: To a solution of 1-(4-(4-(dimethoxymethyl)piperidin-1-yl)phenyl)-6-methoxy-4-methyl-1,2,3,4-tetrahydronaphthalen-1-ol (3 g, 7.05 mmol, 1 eq.) in HCV/MeOH (50 mL) was added. The mixture was stirred at 20° C. for 1 hour. LCMS showed desired compound was detected. The mixture was adjusted to pH 6-7 with NaHCO₃. And then water (80 mL) was added into the mixture, which was extracted with EtOAc (50 mL×3). The combined organic layers were dried over Na₂SO₄, filtered and concentrated to yield 4-(dimethoxymethyl)-1-(4-(6-methoxy-4-methyl-3,4-dihydronaphthalen-1-yl)phenyl)piperidine (2.5 g, 90% purity, 87.02% yield) used for the next step directly.

LC-MS (ESI⁺) m/z: 408.3 (M+H)⁺.

Step 6: To a solution of 4-(dimethoxymethyl)-1-(4-(6-methoxy-4-methyl-3,4-dihydronaphthalen-1-yl)phenyl)piperidine (2.5 g, 6.13 mmol, 1 eq.) in DCM (25 mL) was added Py·HBr₃ (1.77 g, 5.52 mmol, 0.9 eq.). The mixture was stirred at 20° C. for 0.2 hour. LCMS showed desired compound was detected. The reaction mixture was extracted with DCM (50 mL×3) and concentrated to get crude product, which was purified on silica gel column chromatography (from PE/EtOAc=3/1 to 2/1, TLC: PE/EtOAc=3/1, Rf=0.58) to yield 1-(4-(2-bromo-6-methoxy-4-methyl-3,4-dihydronaphthalen-1-yl)phenyl)-4-(dimethoxymethyl)piperidine (1.7 g, 87% purity, 56.97% yield) as a white solid.

LC-MS (ESI⁺) m/z: 488.2 (M+H⁺+2).

Step 7: A mixture of 1-(4-(2-bromo-6-methoxy-4-methyl-3,4-dihydronaphthalen-1-yl)phenyl)-4-(dimethoxymethyl)piperidine (1.5 g, 3.08 mmol, 1 eq.), phenylboronic acid (451.18 mg, 3.70 mmol, 1.2 eq.), Pd(dtbpf)Cl₂ (200.97 mg, 308.36 μmol, 0.1 eq.) and Na₂CO₃ (980.49 mg, 9.25 mmol, 3 eq.) in dioxane (40 mL) and H₂O (8 mL) was degassed and purged with N₂ for 3 times, and then the mixture was stirred at 90° C. for 3 hour under N₂ atmosphere. LCMS showed desired compound was detected. The mixture was extracted with EtOAc (50 mL×3), dried over Na₂SO₄, filtered and concentrated to get crude product, which was purified on silica gel column chromatography (from PE/EtOAc=3/1 to 2/1, TLC: PE/EtOAc=3/1, Rf=0.54) to yield 4-(dimethoxymethyl)-1-(4-(6-methoxy-4-methyl-2-phenyl-3,4-dihydronaphthalen-1-yl)phenyl)piperidine (1.2 g, 97% purity, 80.46% yield) as a pink solid.

LC-MS (ESI⁺) m/z: 484.3 (M+H)⁺.

Step 8: To a solution of 4-(dimethoxymethyl)-1-(4-(6-methoxy-4-methyl-2-phenyl-3,4-dihydronaphthalen-1-yl)phenyl)piperidine (1.2 g, 2.48 mmol, 1 eq.) in EtOAc (30 mL) was added Pd/C (2.64 g, 2.48 mmol, 10% purity, 1 eq.) under H₂ (10.00 mg, 4.96 mmol, 2 eq.) atmosphere. The mixture was stirred under H₂ atmosphere (15 psi) at 25° C. for 16 hour. LCMS showed desired compound was detected. The mixture was filtered and the filtrate was concentrated to yield 4-(dimethoxymethyl)-1-(4-(6-methoxy-4-methyl-2-phenyl-1,2,3,4-tetrahydronaphthalen-1-yl)phenyl)piperidine (1.04 g, crude) as a yellow solid used for the next step directly.

LC-MS (ESI⁺) m/z: 486.5 (M+H)⁺.

Step 9: 4-(dimethoxymethyl)-1-(4-(6-methoxy-4-methyl-2-phenyl-1,2,3,4-tetrahydronaphthalen-1-yl)phenyl)piperidine (1 g, 2.06 mmol, 1 eq.) was separated by chiral SFC (column: DAICEL CHIRALPAK OJ 250 mm*30 mm, 10 um); mobile phase: [CO₂-EtOH (0.1% NH₃H₂O)]; B %: 30%-30%; Flow Rate: 80 ml/min; peak 1:2.944 min; peak 2:3.156 min; peak 3:3.244 min; peak 2:3.756 min) to yield peak 1, peak 2, peak 3 and peak 4.

Peak 1 was concentrated under reduced pressure to yield 4-(dimethoxymethyl)-1-(4-((1R,2S,4R)-6-methoxy-4-methyl-2-phenyl-1,2,3,4-tetrahydronaphthalen-1-yl)phenyl)piperidine (250 mg, 25.00% yield) as a yellow solid. LC-MS (ESI⁺) m/z: 486.4 (M+H)⁺.

Peak 2 was concentrated under reduced pressure to yield 4-(dimethoxymethyl)-1-(4-((1S,2R,4R)-6-methoxy-4-methyl-2-phenyl-1,2,3,4-tetrahydronaphthalen-1-yl)phenyl)piperidine (30 mg, 3.00% yield) as a yellow solid. LC-MS (ESI⁺) m/z: 486.4 (M+H)⁺.

Peak 3 was concentrated under reduced pressure to yield 4-(dimethoxymethyl)-1-(4-((1R,2S,4S)-6-methoxy-4-methyl-2-phenyl-1,2,3,4-tetrahydronaphthalen-1-yl)phenyl)piperidine (20 mg, 2.00% yield) as a yellow solid. LC-MS (ESI⁺) m/z: 486.4 (M+H)⁺.

Peak 4 was concentrated under reduced pressure to yield 4-(dimethoxymethyl)-1-(4-((1S,2R,4S)-6-methoxy-4-methyl-2-phenyl-1,2,3,4-tetrahydronaphthalen-1-yl)phenyl)piperidine (150 mg, 15.00% yield) as a yellow solid. LC-MS (ESI⁺) m/z: 486.4 (M+H)⁺.

Step 10: 4-(dimethoxymethyl)-1-(4-(1R,2S,4R)-6-methoxy-4-methyl-2-phenyl-1,2,3,4-tetrahydronaphthalen-1-yl)phenyl)piperidine (100 mg, 205.91 μmol, 1 eq.) was added in DCM (10 mL), then BBr₃ (154.75 mg, 617.72 μmol, 59.52 uL, 3 eq.) was added in the mixture with stirring at 0° C. After 30 min, the reaction mixture was quenched by the addition of water (10 mL) at 0° C. The mixture was stirred at 20° C. for 2 hour. After completion, the mixture was concentrated under reduced pressure to give 1-(4-((1R,2S,4R)-6-hydroxy-4-methyl-2-phenyl-1,2,3,4-tetrahydronaphthalen-1-yl)phenyl)piperidine-4-carbaldehyde (80 mg, crude) as a yellow solid without purification.

LC-MS (ESI⁺) m/z: 426.3 (M+H)⁺.

Step 11:4-(dimethoxymethyl)-1-(4-((1S,2R,4R)-6-methoxy-4-methyl-2-phenyl-1,2,3,4-tetrahydronaphthalen-1-yl)phenyl)piperidine (30.00 mg, 61.77 μmol, 1 eq.) was added in DCM (5 mL), then BBr₃ (46.43 mg, 185.32 μmol, 17.86 uL, 3 eq.) was added in the mixture with stirring at 0° C. After 30 min, the reaction mixture was quenched by the addition of water (10 mL) at 0° C. The mixture was stirred at 20° C. for 2 hour. After completion, the mixture was concentrated under reduced pressure to give 1-(4-((1S,2R,4R)-6-hydroxy-4-methyl-2-phenyl-1,2,3,4-tetrahydronaphthalen-1-yl)phenyl)piperidine-4-carbaldehyde (30 mg, crude) as a yellow solid without purification.

LC-MS (ESI+) m/z: 426.3 (M+H)⁺.

Step 12: 4-(dimethoxymethyl)-1-(4-((1R,2S,4S)-6-methoxy-4-methyl-2-phenyl-1,2,3,4-tetrahydronaphthalen-1-yl)phenyl)piperidine (20.00 mg, 41.18 μmol, 1 eq.) was added in DCM (5 mL), then BBr₃ (30.95 mg, 123.54 μmol, 11.90 μL, 3 eq.) was added in the mixture with stirring at 0° C. After 30 min, the reaction mixture was quenched by the addition of water (10 mL) at 0° C. The mixture was stirred at 20° C. for 2 hour. After completion, the mixture was concentrated under reduced pressure to give 1-(4-((1R,2S,4S)-6-hydroxy-4-methyl-2-phenyl-1,2,3,4-tetrahydronaphthalen-1-yl)phenyl)piperidine-4-carbaldehyde (20 mg, crude) as a yellow solid without purification.

LC-MS (ESI+) m/z: 426.3 (M+H)⁺.

Step 13: 4-(dimethoxymethyl)-1-(4-((1S,2R,4S)-6-methoxy-4-methyl-2-phenyl-1,2,3,4-tetrahydronaphthalen-1-yl)phenyl)piperidine (100.00 mg, 205.91 μmol, 1 eq.) was added in DCM (10 mL), then BBr₃ (154.75 mg, 617.72 μmol, 59.52 uL, 3 eq.) was added in the mixture with stirring at 0° C. After 30 min, the reaction mixture was quenched by the addition of water (10 mL) at 0° C. The mixture was stirred at 20° C. for 2 hour. After completion, the mixture was concentrated under reduced pressure to give 1-(4-((1S,2R,4S)-6-hydroxy-4-methyl-2-phenyl-1,2,3,4-tetrahydronaphthalen-1-yl)phenyl)piperidine-4-carbaldehyde (100 mg, crude) as a yellow solid without purification.

LC-MS (ESI+) m/z: 426.3 (M+H)⁺

Step 14: To a solution of 1-(4-((1R,2S,4R)-6-hydroxy-4-methyl-2-phenyl-1,2,3,4-tetrahydronaphthalen-1-yl)phenyl)piperidine-4-carbaldehyde (80 mg, 187.99 μmol, 1 eq.) and (S)-3-(1-oxo-5-(piperazin-1-yl)isoindolin-2-yl)piperidine-2,6-dione (61.73 mg, 187.99 μmol, 1 eq.) in DCE (5 mL) was added DIEA (24.30 mg, 187.99 μmol, 32.74 μL, 1 eq.). After addition, the mixture was stirred at 25° C. for 30 min, and then NaBH(OAc); (119.53 mg, 563.96 μmol, 3 eq.) was added. The mixture was stirred at 25° C. for 3 hour. After completion, the reaction mixture was concentrated and purified by preparative HPLC(Column: C18-1 150*30 mm*5 um; Condition water (NH₄HCO₃)-CAN, B %: 58%-88%, 11 min) followed by lyophilization to yield (S)-3-(5-(4-((1-(4-((1R,2S,4R)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (23.7 mg, 32.12 μmol, 17.08% yield, 97.76% purity) as a white solid. LC-MS (ESI⁺) m/z: 738.6 (M+H)⁺.

LCMS: calc. for C₄₆H₅₁N₅O₄:737.95, found: [M+H]⁺ 738.6.

HPLC: 97.76% purity at 220 nm.

¹H NMR: ¹H NMR (400 MHZ, CHLOROFORM-d) δ ppm 7.87-7.91 (1H, m), 7.72-7.78 (1H, m), 7.17 (2H, s), 7.01 (1H, dd, J=8.34, 1.79 Hz), 6.89-6.94 (2H, m), 6.83-6.87 (3H, m), 6.60-6.63 (3H, m), 6.29 (2H, d, J=8.58 Hz), 5.19-5.26 (1H, m), 4.59-4.89 (1H, m), 4.26-4.46 (2H, m), 4.21-4.24 (1H, m), 3.56-3.62 (2H, m), 3.38-3.43 (1H, m), 3.32-3.36 (4H, m), 3.08-3.15 (1H, m), 2.79-2.99 (3H, m), 2.61-2.64 (1H, m), 2.57-2.60 (5H, m), 2.35 (1H, dd, J=13.11, 5.01 Hz), 2.28 (2H, br d, J=7.39 Hz), 2.17-2.24 (1H, m), 1.90-1.95 (1H, m), 1.83-1.89 (3H, m), 1.49-1.52 (3H, m), 1.31-1.38 (2H, m).

Step 15: To a solution of 1-(4-(1S,2R,4R)-6-hydroxy-4-methyl-2-phenyl-1,2,3,4-tetrahydronaphthalen-1-yl)phenyl)piperidine-4-carbaldehyde (30.00 mg, 70.50 μmol, 1 eq.) and (S)-3-(1-oxo-5-(piperazin-1-yl)isoindolin-2-yl)piperidine-2,6-dione (23.15 mg, 70.50 μmol, 1 eq.) in DCE (5 mL) was added DIEA (9.11 mg, 70.50 μmol, 12.28 uL, 1 eq.). After addition, the mixture was stirred at 25° C. for 30 min, and then NaBH(OAc) 3 (44.82 mg, 211.49 μmol, 3 eq.) was added. The mixture was stirred at 25° C. for 3 hour. After completion, the reaction mixture was concentrated and purified by preparative HPLC(Column: C18-1 150*30 mm*5 um; Condition water (NH₄HCO₃)-CAN, B %: 58%-88%, 11 min) followed by lyophilization to yield (S)-3-(5-(4-((1-(4-((1S,2R,4R)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (14.7 mg, 19.92 μmol, 28.26% yield, 93.06% purity) as a white solid.

LC-MS (ESI⁺) m/z: 738.6 (M+H)⁺.

LCMS: calc. for C₄₆H5N₅O₄: 737.95, found: [M+H]⁺ 738.6.

HPLC: 93.06% purity at 220 nm.

¹H NMR: ¹H NMR (400 MHZ, CHLOROFORM-d) δ ppm 7.90-7.98 (1H, m), 7.71-7.80 (1H, m), 7.17 (2H, s), 6.99-7.04 (1H, m), 6.85-6.90 (2H, m), 6.76-6.84 (3H, m), 6.56-6.61 (3H, m), 6.24-6.29 (2H, m), 5.17-5.26 (1H, m), 4.25-4.46 (2H, m), 4.18-4.23 (1H, m), 3.52-3.60 (3H, m), 3.35 (4H, br s), 3.24-3.29 (1H, m), 2.82-2.94 (2H, m), 2.60 (5H, br d, J=8.58 Hz), 2.56 (1H, br s), 2.35-2.47 (2H, m), 2.28-2.32 (2H, m), 2.21-2.25 (1H, m), 1.86 (2H, br d, J=13.11 Hz), 1.62-1.69 (1H, m), 1.56-1.60 (1H, m), 1.39-1.43 (3H, m), 1.35 (2H, br d, J=11.92 Hz).

Step 16: To a solution of 1-(4-((1R,2S,4S)-6-hydroxy-4-methyl-2-phenyl-1,2,3,4-tetrahydronaphthalen-1-yl)phenyl)piperidine-4-carbaldehyde (20.00 mg, 47.00 μmol, 1 eq.) and (S)-3-(1-oxo-5-(piperazin-1-yl)isoindolin-2-yl)piperidine-2,6-dione (15.43 mg, 47.00 μmol, 1 eq.) in DCE (5 mL) was added DIEA (6.07 mg, 47.00 μmol, 8.19 uL, 1 eq.). After addition, the mixture was stirred at 25° C. for 30 min, and then NaBH(OAc)₃ (29.88 mg, 140.99 μmol, 3 eq.)

was added. The mixture was stirred at 25° C. for 3 hour. After completion, the reaction mixture was concentrated and purified by preparative HPLC(Column: C18-1 150*30 mm*5 um; Condition water (NH₄HCO₃)-CAN, B %: 60%-90%, 11 min) followed by lyophilization to yield (S)-3-(5-(4-((1-(4-((1R,2S,4S)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (8.1 mg, 10.98 μmol, 23.36% yield, 98.91% purity) as a white solid.

LC-MS (ESI⁺) m/z: 738.6 (M+H)⁺.

LCMS: calc. for C₄₆H51N₅O₄: 737.95, found: [M+H]⁺ 738.6.

HPLC: 98.91% purity at 220 nm.

¹H NMR: ¹H NMR (400 MHZ, CHLOROFORM-d) δ ppm 7.80-7.86 (1H, m), 7.60-7.70 (1H, m), 7.07-7.09 (2H, m), 6.90-6.96 (1H, m), 6.76-6.81 (2H, m), 6.75 (2H, s), 6.67-6.69 (1H, m), 6.47-6.52 (3H, m), 6.15-6.19 (2H, m), 5.04-5.21 (1H, m), 4.36 (2H, s), 4.09-4.13 (1H, m), 3.44-3.52 (3H, m), 3.22-3.29 (4H, m), 3.16 (1H, br s), 2.71-2.86 (2H, m), 2.45-2.54 (6H, m), 2.26 (2H, s), 2.19-2.23 (2H, m), 2.10-2.16 (1H, m), 1.75-1.80 (2H, m), 1.53-1.59 (1H, m), 1.47 (1H, s), 1.30-1.33 (3H, m), 1.20-1.29 (2H, m).

Step 17: To a solution of 1-(4-((1S,2R,4S)-6-hydroxy-4-methyl-2-phenyl-1,2,3,4-tetrahydronaphthalen-1-yl)phenyl)piperidine-4-carbaldehyde (80.00 mg, 187.99 μmol, 1 eq.) and (S)-3-(1-oxo-5-(piperazin-1-yl)isoindolin-2-yl)piperidine-2,6-dione (61.73 mg, 187.99 μmol, 1 eq.) in DCE (5 mL) was added DIEA (24.30 mg, 187.99 μmol, 32.74 μL, 1 eq.). After addition, the mixture was stirred at 25° C. for 30 min, and then NaBH(OAc)₃ (119.53 mg, 563.96 μmol, 3 eq.) was added. The mixture was stirred at 25° C. for 3 hour. After completion, the reaction mixture was concentrated and purified by preparative HPLC(Column: C18-1 150 $30 mm*5 um; Condition water (NH₄HCO₃)-CAN, B %: 60%-90%, 11 min) followed by lyophilization to yield (S)-3-(5-(4-((1-(4-((1S,2R,4S)-6-hydroxy-4-methyl-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione (29.2 mg, 39.57 μmol, 21.05% yield, 96.74% purity) as a white solid. LC-MS (ESI⁺) m/z: 738.6 (M+H)⁺.

LCMS: calc. for C₄₆H₅₁N₅O₄: 737.95, found: [M+H]⁺ 738.6.

HPLC: 96.74% purity at 220 nm.

¹H NMR: ¹H NMR (400 MHZ, CHLOROFORM-d) δ ppm 7.88-7.99 (1H, m), 7.74 (1H, d, J=8.46 Hz), 7.16-7.18 (2H, m), 6.98-7.02 (1H, m), 6.87-6.92 (2H, m), 6.83 (3H, dd, J=5.30, 2.80 Hz), 6.55-6.62 (3H, m), 6.27 (2H, d, J=8.46 Hz), 5.21 (1H, dd, J=13.17, 5.07 Hz), 4.78 (1H, s), 4.24-4.44 (2H, m), 4.19-4.23 (1H, m), 3.54-3.61 (2H, m), 3.36-3.41 (1H, m), 3.32 (4H, br s), 3.05-3.13 (1H, m), 2.89-2.96 (1H, m), 2.78-2.88 (1H, m), 2.60-2.63 (1H, m), 2.54-2.60 (5H, m), 2.33 (1H, br dd, J=13.35, 4.89 Hz), 2.27 (2H, br d, J=6.32 Hz), 2.17-2.24 (1H, m), 1.91 (1H, br d, J=12.04 Hz), 1.86 (3H, br d, J=8.58 Hz), 1.48 (3H, d, J=6.79 Hz), 1.29-1.37 (2H, m).

The stereochemistry of the ERBM moieties for all four molecules was arbitrarily assigned

### Example 252. Preparation of (I-204) 3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-3-methyl-2-oxo-2,3-dihydro-1H-benzo[d]imidazol-1-yl)piperidine-2,6-dione

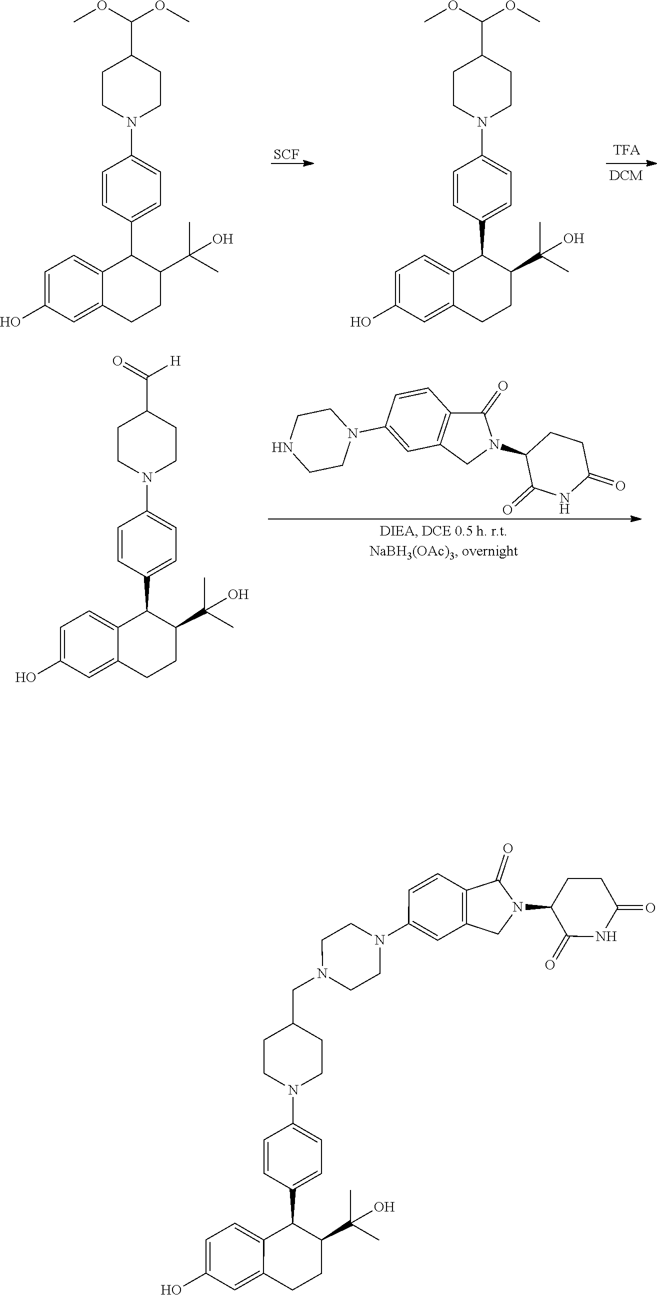

Step 1: 5-(4-(4-(dimethoxymethyl)piperidin-1-yl)phenyl)-6-(2-hydroxypropan-2-yl)-5,6,7,8-tetrahydronaphthalen-2-ol (400 mg, 758.02 μmol, 1 eq.) was separated by SFC. LCMS showed desired compound was detected. The crude product was purified on silica gel column chromatography (from PE/EtOAc=1/1 to 2/1, TLC: PE/EtOAc=3/1, Rf=0.45) to yield desired compound as brown oil, which was separated by chiral SFC (column: DAICEL CHIRALPAK AD (250 mm*30 mm, 10 um); mobile phase: [0.1% NH₃H₂O IPA]; B %: 55%-55%, 45 min, SFC: Rt=1.967, 2.372) to yield (5R,6S)-5-(4-(4-(dimethoxymethyl)piperidin-1-yl)phenyl)-6-(2-hydroxypropan-2-yl)-5,6,7,8-tetrahydronaphthalen-2-ol (100 mg, 227.49 μmol, 30.01% yield) as white solid.

LC-MS (ESI⁺) m/z: 440.3 (M+H)⁺.

Step 2: To a solution of (5R,6S)-5-(4-(4-(dimethoxymethyl)piperidin-1-yl)phenyl)-6-(2-hydroxypropan-2-yl)-5,6,7,8-tetrahydronaphthalen-2-ol (100.00 mg, 227.49 μmol, 1 eq.) in DCM (6 mL) was added TFA (259.38 mg, 2.27 mmol, 168.98 μL, 10 eq.). The mixture was stirred at 25° C. for 2 hr. LC-MS showed one main peak with desired mass was detected. The reaction mixture or solution was concentrated to dryness. 1-(4-((1R,2S)-6-hydroxy-2-(2-hydroxypropan-2-yl)-1,2,3,4-tetrahydronaphthalen-1-yl)phenyl)piperidine-4-carbaldehyde (80 mg, 203.29 μmol, 89.37% yield) as yellow liquid was used into the next step without further purification.

LC-MS (ESI⁺) m/z: 394.2 (M+H)⁺.

Step 3: To a solution of 1-(4-((1R,2S)-6-hydroxy-2-(2-hydroxypropan-2-yl)-1,2,3,4-tetrahydronaphthalen-1-yl)phenyl)piperidine-4-carbaldehyde (80 mg, 203.29 μmol, 1 eq.) and (S)-3-(1-oxo-5-(piperazin-1-yl)isoindolin-2-yl)piperidine-2,6-dione (80.11 mg, 243.95 μmol, 1.2 eq.) in DCE (20 mL) was added sodium; triacetoxyboranuide (129.26 mg, 609.88 μmol, 3 eq.) and DIPEA (52.55 mg, 406.59 μmol, 70.82 μL, 2 eq.). The mixture was stirred at 25° C. for 5 hr. LC-MS showed one main peak with desired mass was detected. After quenching the reaction, the reaction mixture was poured into separatory funnel and separated. The reaction mixture was partitioned between DCM (100 mL×2) and water (100 mL). The residue was purified by prep-HPLC(Column C18-1 150*30 mm*5 um, Condition water (NH₄HCO₃)-CAN, Begin B 40, End B 70, Gradient Time (min) 11, 100% B Hold Time (min) 2, FlowRate (ml/min) 25, Injections 5, HPLC 95). (S)-3-(5-(4-((1-(4-((1R,2S)-6-hydroxy-2-(2-hydroxypropan-2-yl)-1,2,3,4-tetrahydronaphthalen-1-yl)phenyl)piperidin-4-yl)methyl)piperazin-1-yl)-1-oxoisoindolin-2-yl)piperidine-2,6-dione (10.6 mg, 15.02 μmol, 7.39% yield) was obtained as a white solid.

LC-MS (ESI⁺) m/z: 706.4 (M+H)⁺.

LCMS: calc. for C₄₂H₅₁N₅O₅: 705.39, found: [M+H]⁺ 706.4.

HPLC: 100.00% purity at 220 nm.

¹H NMR: ¹H NMR (400 MHZ, METHANOL-d₄) δ ppm 7.61-7.72 (m, 1H) 7.08-7.15 (m, 2H) 6.99-7.08 (m, 2H) 6.85-6.93 (m, 2H) 6.63-6.70 (m, 1H) 6.55-6.60 (m, 1H) 6.42-6.49 (m, 1H) 5.08-5.17 (m, 1H) 4.36-4.47 (m, 2H) 4.22-4.29 (m, 1H) 3.58-3.68 (m, 2H) 3.41 (br s, 4H) 2.99-3.05 (m, 1H) 2.86-2.90 (m, 1H) 2.81 (d, J=2.62 Hz, 1H) 2.67-2.72 (m, 4H) 2.38-2.41 (m, 1H) 2.14-2.20 (m, 1H) 2.05 (br d, J=3.46 Hz, 6H) 1.63-1.77 (m, 1H) 1.31 (br s, 6H) 1.05-1.13 (m, 3H) 0.87-0.96 (m, 1H) 0.73-0.82 (m, 3H).

### Example 253. Preparation of (I-219) (S)-3-(5-(4-((1-(4-((1S,2R)-6-hydroxy-2-(2-hydroxypropan-2-yl)-1,2,3,4-tetrahydronaphthalen-1-yl)phenyl)piperidin-4-yl)methyl)piperazin-1-yl)-1-oxoisoindolin-2-yl)piperidine-2,6-dione

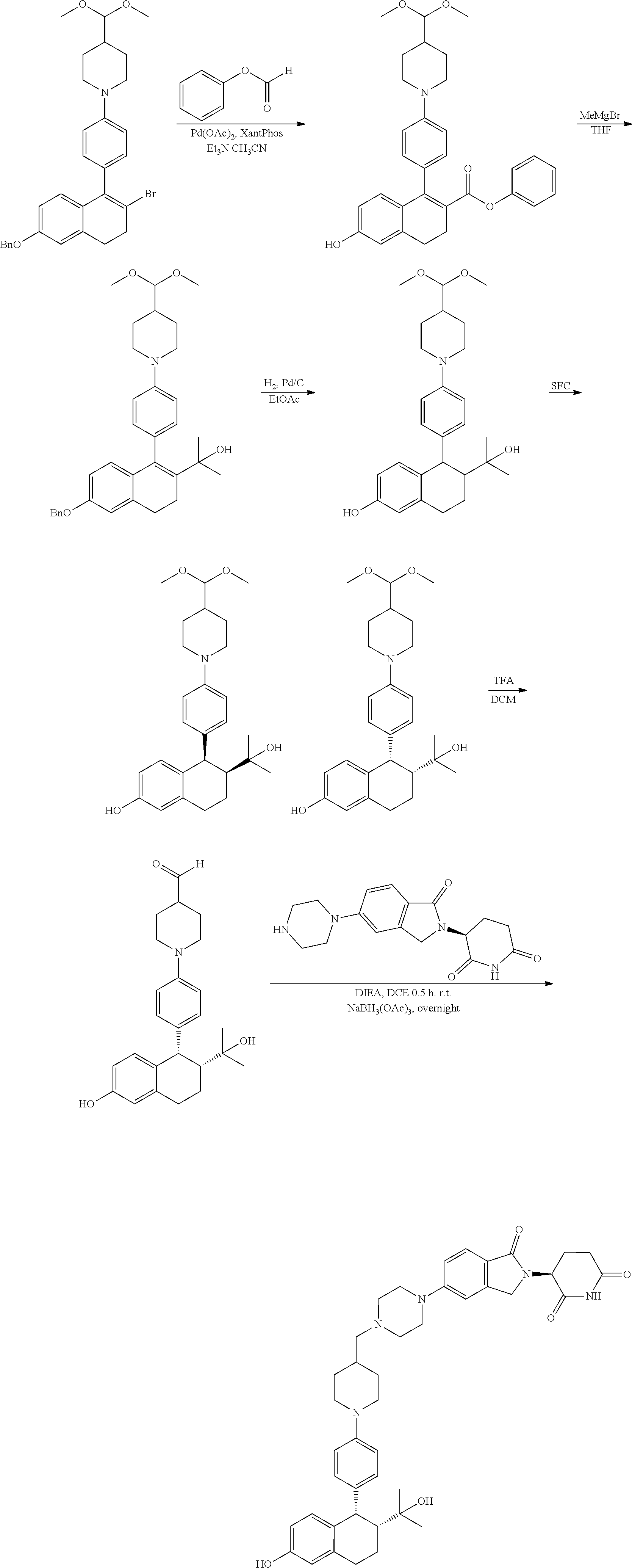

Step 1: To a solution of 1-(4-(6-(benzyloxy)-2-bromo-3,4-dihydronaphthalen-1-yl)phenyl)-4-(dimethoxymethyl)piperidine (1.5 g, 2.73 mmol, 1 eq.) and phenyl formate (667.93 mg, 5.47 mmol, 596.36 uL, 2 eq.) in MeCN (10 mL) was added Xantphos (395.59 mg, 683.67 μmol, 0.25 eq.), TEA (830.17 mg, 8.20 mmol, 1.14 mL, 3 eq.) and Pd(OAc) 2 (92.09 mg, 410.20 μmol, 0.15 eq.). The mixture was stirred at 80° C. for 6 hr. LC-MS showed one main peak with desired mass was detected. The reaction mixture was partitioned between EA (50 mL×2) and water (50 mL). After quenching the reaction, the reaction mixture was poured into separatory funnel and separated. The crude product was purified by silica gel chromatography eluted with PE:EtOAc=2: 1˜1:1. phenyl 6-(benzyloxy)-1-(4-(4-(dimethoxymethyl)piperidin-1-yl)phenyl)-3,4-dihydronaphthalene-2-carboxylate (1.5 g, 2.54 mmol, 93.01% yield) was obtained as a white solid.

LC-MS (ESI⁺) m/z: 590.3 (M+H)⁺.

Step 2: To a solution of phenyl 6-(benzyloxy)-1-(4-(4-(dimethoxymethyl)piperidin-1-yl)phenyl)-3,4-dihydronaphthalene-2-carboxylate (1 g, 1.70 mmol, 1 eq.) in THF (20 mL) was added bromo(methyl)magnesium (3 M, 5.65 mL, 10 eq.). The mixture was stirred at 0° C. for 5 hr. LC-MS showed one main peak with desired mass was detected. The reaction mixture or solution was concentrated to dryness. The crude product was purified by silica gel chromatography eluted with PE:EtOAc=2: 1˜1:1. 2-(6-(benzyloxy)-1-(4-(4-(dimethoxymethyl)piperidin-1-yl)phenyl)-3,4-dihydronaphthalen-2-yl)propan-2-ol (400 mg, 758.02 μmol, 44.70% yield) as brown solid was used into the next step without further purification.

LC-MS (ESI⁺) m/z: 528.4 (M+H)⁺.

Step 3: The mixture of 2-(6-(benzyloxy)-1-(4-(4-(dimethoxymethyl)piperidin-1-yl)phenyl)-3,4-dihydronaphthalen-2-yl)propan-2-ol (300 mg, 568.51 μmol, 1 eq.), Palladium (60.50 mg, 56.85 μmol, 10% purity, 0.1 eq.) in EtOAc (20 mL) was degassed and the mixture was degassed and refilled with H₂ twice. The reaction mixture was stirred at 50° C. on 50 psi with H₂ for 10 hours. LC-MS showed one main peak with desired mass was detected. The reaction mixture or solution was concentrated to dryness. The crude product was purified by silica gel chromatography eluted with PE:EtOAc=2: 1˜1:1. 5-(4-(4-(dimethoxymethyl)piperidin-1-yl)phenyl)-6-(2-hydroxypropan-2-yl)-5,6,7,8-tetrahydronaphthalen-2-ol (120 mg, 272.98 μmol, 48.02% yield) was obtained as a white solid.

LC-MS (ESI⁺) m/z: 440.3 (M+H)⁺.

Step 4: 5-(4-(4-(dimethoxymethyl)piperidin-1-yl)phenyl)-6-(2-hydroxypropan-2-yl)-5,6,7,8-tetrahydronaphthalen-2-ol (400 mg, 758.02 μmol, 1 eq.) was separated by SFC. LCMS showed desired compound was detected. The crude product was purified on silica gel column chromatography (from PE/EtOAc=1/1 to 2/1, TLC: PE/EtOAc=3/1, Rf=0.45) to yield desired compound as brown oil, which was separated by chiral SFC (column: DAICEL CHIRALPAK AD (250 mm*30 mm, 10 um); mobile phase: [0.1% NH₃H₂O IPA]; B %: 55%-55%, 45 min, SFC: Rt=1.967, 2.372) to yield (5R,6S)-5-(4-(4-(dimethoxymethyl)piperidin-1-yl)phenyl)-6-(2-hydroxypropan-2-yl)-5,6,7,8-tetrahydronaphthalen-2-ol (100 mg, 227.49 μmol, 30.01% yield) as white solid and (5S,6R)-5-(4-(4-(dimethoxymethyl)piperidin-1-yl)phenyl)-6-(2-hydroxypropan-2-yl)-5,6,7,8-tetrahydronaphthalen-2-ol (110 mg, 250.24 μmol, 33.01% yield) as white solid.

LC-MS (ESI⁺) m/z: 440.3 (M+H)⁺.

Step 5: To a solution of (5S,6R)-5-(4-(4-(dimethoxymethyl)piperidin-1-yl)phenyl)-6-(2-hydroxypropan-2-yl)-5,6,7,8-tetrahydronaphthalen-2-ol (110 mg, 250.24 μmol, 1 eq) in DCM (6 mL) was added TFA (285.32 mg, 2.50 mmol, 185.88 uL, 10 eq.). The mixture was stirred at 25° C. for 2 hr. LC-MS showed one main peak with desired mass was detected. The reaction mixture or solution was concentrated to dryness. The crude product 1-(4-((1S,2R)-6-hydroxy-2-(2-hydroxypropan-2-yl)-1,2,3,4-tetrahydronaphthalen-1-yl)phenyl)piperidine-4-carbaldehyde (80 mg, 203.29 μmol, 81.24% yield) as yellow liquid was used into the next step without further purification.

LC-MS (ESI⁺) m/z: 394.2 (M+H)⁺.

Step 6: To a solution of 1-(4-((1S,2R)-6-hydroxy-2-(2-hydroxypropan-2-yl)-1,2,3,4-tetrahydronaphthalen-1-yl)phenyl)piperidine-4-carbaldehyde (80 mg, 203.29 μmol, 1 eq.) and (S)-3-(1-oxo-5-(piperazin-1-yl)isoindolin-2-yl)piperidine-2,6-dione (80.11 mg, 243.95 μmol, 1.2 eq.) in DCE (20 mL) was added sodium; triacetoxyboranuide (129.26 mg, 609.88 μmol, 3 eq.) and DIPEA (52.55 mg, 406.59 μmol, 70.82 μL, 2 eq.). The mixture was stirred at 25° C. for 5 hr. LC-MS showed one main peak with desired mass was detected. After quenching the reaction, the reaction mixture was poured into separatory funnel and separated. The reaction mixture was partitioned between DCM (100 mL×2) and water (100 mL). The residue was purified by prep-HPLC(Column C18-1 150*30 mm*5 um Condition water (NH₄HCO₃)-CAN, Begin B 40, End B 70, Gradient Time (min) 11, 100% B Hold Time (min) 2, FlowRate (ml/min) 25, Injections 5, HPLC 95). (S)-3-(5-(4-((1-(4-((1S,2R)-6-hydroxy-2-(2-hydroxypropan-2-yl)-1,2,3,4-tetrahydronaphthalen-1-yl)phenyl)piperidin-4-yl)methyl)piperazin-1-yl)-1-oxoisoindolin-2-yl)piperidine-2,6-dione (6.7 mg, 9.49 μmol, 4.67% yield) was obtained as a white solid. LC-MS (ESI⁺) m/z: 706.4 (M+H)⁺.

LCMS: calc. for C₄₂H₅₁N₅O₅: 705.39, found: [M+H]⁺ 706.4.

HPLC: 100.00% purity at 220 nm.

¹H NMR: ¹H NMR (400 MHZ, METHANOL-d₄) δ ppm 7.64 (d, J=8.34 Hz, 1H) 7.06-7.12 (m, 2H) 7.03 (d, J=8.58 Hz, 2H) 6.86 (d, J=8.70 Hz, 2H) 6.65 (d, J=8.34 Hz, 1H) 6.53-6.58 (m, 1H) 6.41-6.47 (m, 1H) 5.10 (dd, J=13.35, 5.13 Hz, 1H) 4.35-4.45 (m, 2H) 4.24 (br d, J=3.46 Hz, 1H) 3.58-3.65 (m, 2H) 3.36-3.40 (m, 4H) 2.96-3.03 (m, 1H) 2.85-2.89 (m, 1H) 2.66 (br d, J=5.84 Hz, 4H) 2.36 (br d, J=7.03 Hz, 1H) 2.17-2.22 (m, 1H) 1.86-2.05 (m, 6H) 1.61-1.74 (m, 1H) 1.29-1.37 (m, 7H) 1.07 (s, 3H) 0.88-0.93 (m, 1H) 0.76 (s, 3H).

### Example 254. Preparation of (I-154) (S)-3-(5-(4-((1-(5-((1S,2R)-2-cyclohexyl-6-hydroxy-1,2,3,4-tetrahydronaphthalen-1-yl)pyridin-2-yl)piperidin-4-yl)methyl)piperazin-1-yl)-1-oxoisoindolin-2-yl)piperidine-2,6-dione and (I-155) (S)-3-(5-(4-((1-(5-((1R,2S)-2-cyclohexyl-6-hydroxy-1,2,3,4-tetrahydronaphthalen-1-yl)pyridin-2-yl)piperidin-4-yl)methyl)piperazin-1-yl)-1-oxoisoindolin-2-yl)piperidine-2,6-dione

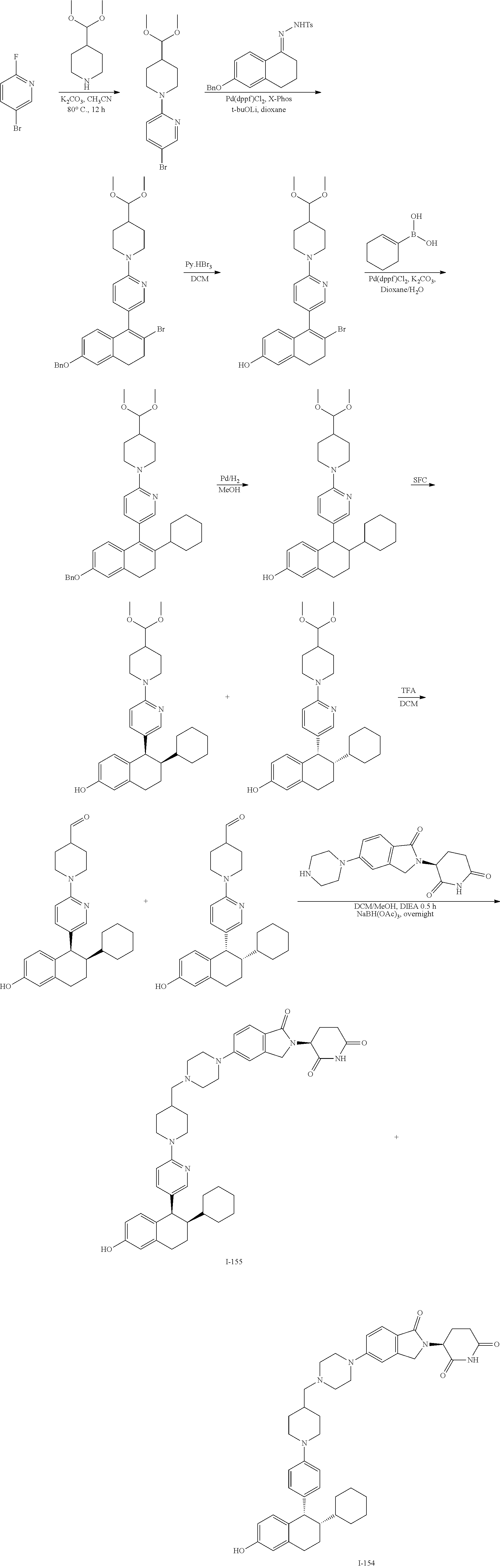

Step 1: To a solution of 5-bromo-2-fluoropyridine (1.30 g, 8.18 mmol, 1.2 eq.) and 4-(dimethoxymethyl)piperidine (1.2 g, 6.82 mmol, 701.75 μL, 1 eq.) in CH₃CN (50 mL) were added K₂CO₃ (1.88 g, 13.64 mmol, 2 eq.). The mixture was stirred at 80° C. for 16 hr. The mixture was stirred at 80° C. for 16 hr. LCMS showed the ˜93% desired MS was detected. The reaction mixture was extracted with EtOAc (40 mL×3) and concentrated to get crude product. The crude product was purified on silica gel column chromatography (from PE/EtOAc=3/1 to 2/1, TLC: PE/EtOAc=3/1, Rf=0.7) to yield desired 5-bromo-2-(4-(dimethoxymethyl)piperidin-1-yl)pyridine (1.2 g, 91% purity) as a white solid.

LC-MS (ESI⁺) m/z: 316.8 (M+H⁺+2).

Step 2: A mixture of v (1.2 g, 3.81 mmol, 1 eq.), (E)-N′-(6-(benzyloxy)-3,4-dihydronaphthalen-1 (2H)-ylidene)-4-methylbenzenesulfonohydrazide (1.76 g, 4.19 mmol, 1.1 eq.), Pd(dppf)Cl₂ (278.56 mg, 380.70 μmol, 0.1 eq.), X-Phos (90.74 mg, 190.35 μmol, 0.05 eq.) and t-BuOLi (914.31 mg, 11.42 mmol, 1.03 mL, 3 eq.) in dioxane (80 mL) was degassed and purged with N₂ for 3 times, and then the mixture was stirred at 100° C. for 16 h under N₂ atmosphere. LCMS showed the desired MS was detected. The reaction mixture was concentrated under reduced pressure to remove dioxane to get the crude product, which was purified by silica gel column chromatography (from PE/EtOAc=3/1 to 2/1, TLC: PE/EtOAc=3/1, Rf=0.51) to yield desired 5-(6-(benzyloxy)-3,4-dihydronaphthalen-1-yl)-2-(4-(dimethoxymethyl)piperidin-1-yl)pyridine (0.9 g, 90% purity) as a yellow solid.

LC-MS (ESI⁺) m/z: 471.0 (M+H)⁺.

Step 3: To a solution of v (1 g, 2.12 mmol, 1 eq.) in DCM (20 mL) was added Py·HBr₃ (407.76 mg, 1.27 mmol, 0.6 eq.). The mixture was stirred at 25° C. for 0.1 hr. LCMS showed the desired MS was detected. The reaction mixture was concentrated in vacuum, which was extracted with DCM (80 mL×3) and concentrated to get crude product. The crude product was purified by silica gel chromatography (from PE/EtOAc=3/1 to 2/1, TLC: PE/EtOAc=3/1, Rf=0.38) to give 5-(6-(benzyloxy)-2-bromo-3,4-dihydronaphthalen-1-yl)-2-(4-(dimethoxymethyl)piperidin-1-yl)pyridine (0.83 g, 90% purity) as yellow oil.

LC-MS (ESI⁺) m/z: 551.2 (M+H⁺+2).

Step 4: A mixture of 5-(6-(benzyloxy)-2-bromo-3,4-dihydronaphthalen-1-yl)-2-(4-(dimethoxymethyl)piperidin-1-yl)pyridine (830.00 mg, 1.51 mmol, 1 eq.), cyclohex-1-en-1-ylboronic acid (228.31 mg, 1.81 mmol, 1.2 eq.), Pd(dppf)Cl₂ (110.52 mg, 151.05 μmol, 0.1 eq.) and K₂CO₃ (626.29 mg, 4.53 mmol, 3 eq.) in dioxane/H2O (15 mL) was degassed and purged with N₂ for 3 times, and then the mixture was stirred at 80° C. for 3 hr under N₂ atmosphere. LCMS showed desired compound was detected. The mixture was extracted with EtOAc (30 mL×3), dried over Na₂SO₄, filtered and concentrated to get crude product, which was purified on silica gel column chromatography (from PE/EtOAc=3/1 to 2/1, TLC: PE/EtOAc=2/1, Rf=0.4) to yield 5-(6-(benzyloxy)-2-(cyclohex-1-en-1-yl)-3,4-dihydronaphthalen-1-yl)-2-(4-(dimethoxymethyl)piperidin-1-yl)pyridine (0.7 g, 95% purity) as yellow oil.

LC-MS (ESI⁺) m/z: 551.3 (M+H)⁺.

Step 5: To a solution of 5-(6-(benzyloxy)-2-(cyclohex-1-en-1-yl)-3,4-dihydronaphthalen-1-yl)-2-(4-(dimethoxymethyl)piperidin-1-yl)pyridine (500 mg, 907.89 μmol, 1 eq.) in MeOH (15 mL) was added Pd/C (50 wt %, 428.58 mg, 2.52 mmol, 2.78 eq.) under H₂ atmosphere. The suspension was degassed and purged with H₂ (32.96 mg, 16.32 mmol, 35.95 eq.) for 3 times. The mixture was stirred under H₂ (50 psi) at 50° C. for 12 h. LCMS showed desired compound was detected. The mixture was filtered and the filtrate was concentrated to yield 6-cyclohexyl-5-(6-(4-(dimethoxymethyl)piperidin-1-yl)pyridin-3-yl)-5,6,7,8-tetrahydronaphthalen-2-ol (0.34 g, crude) as a white solid used for the next step directly.

LC-MS (ESI⁺) m/z: 465.4 (M+H)⁺.

Step 6: 6-cyclohexyl-5-(6-(4-(dimethoxymethyl)piperidin-1-yl)pyridin-3-yl)-5,6,7,8-tetrahydronaphthalen-2-ol (340 mg, 731.75 μmol, 1 eq.) was separated by chiral SFC (column: DAICEL CHIRALPAK AD 250 mm*30 mm, 10 um); mobile phase: [CO₂-EtOH (0.1% NH₃H₂O)]; B %: 30%-30%; Flow Rate: 80 ml/min; peak 1:1.685 min; peak 2:2.036 min) to yield peak 1 and peak 2.

Peak 1 was concentrated under reduced pressure to yield (5R,6S)-6-cyclohexyl-5-(6-(4-(dimethoxymethyl)piperidin-1-yl)pyridin-3-yl)-5,6,7,8-tetrahydronaphthalen-2-ol (120 mg, 35.29% yield) as yellow oil. LC-MS (ESI⁺) m/z: 465.4 (M+H)⁺.

Peak 2 was concentrated under reduced pressure to yield (5S,6R)-6-cyclohexyl-5-(6-(4-(dimethoxymethyl)piperidin-1-yl)pyridin-3-yl)-5,6,7,8-tetrahydronaphthalen-2-ol (80 mg, 23.53% yield) as yellow oil. LC-MS (ESI⁺) m/z: 465.4 (M+H)⁺.

Step 7: To a solution of (5R,6S)-6-cyclohexyl-5-(6-(4-(dimethoxymethyl)piperidin-1-yl)pyridin-3-yl)-5,6,7,8-tetrahydronaphthalen-2-ol (120.00 mg, 258.27 μmol, 1 eq.) in DCM (12 mL) was added TFA (6.14 g, 53.85 mmol, 4.00 mL, 208.51 eq.). The mixture was stirred at 25° C. for 12 hr. LCMS showed desired compound was detected. After completion, the mixture was concentrated under reduced pressure to give 1-(5-((1R,2S)-2-cyclohexyl-6-hydroxy-1,2,3,4-tetrahydronaphthalen-1-yl)pyridin-2-yl)piperidine-4-carbaldehyde (80 mg, crude) as a white solid without purification.

LC-MS (ESI⁺) m/z: 419.4 (M+H)⁺.

Step 8: To a solution of (5S,6R)-6-cyclohexyl-5-(6-(4-(dimethoxymethyl)piperidin-1-yl)pyridin-3-yl)-5,6,7,8-tetrahydronaphthalen-2-ol (80.00 mg, 172.18 μmol, 1 eq.) in DCM (6 mL) was added TFA (4.09 g, 35.90 mmol, 2.67 mL, 208.51 eq.). The mixture was stirred at 25° C. for 12 hr. LCMS showed desired compound was detected. After completion, the mixture was concentrated under reduced pressure to give 1-(5-((1S,2R)-2-cyclohexyl-6-hydroxy-1,2,3,4-tetrahydronaphthalen-1-yl)pyridin-2-yl)piperidine-4-carbaldehyde (80 mg, crude) as a white solid without purification.

LC-MS (ESI⁺) m/z: 419.4 (M+H)⁺.

Step 9: To a solution of 1-(5-((1R,2S)-2-cyclohexyl-6-hydroxy-1,2,3,4-tetrahydronaphthalen-1-yl)pyridin-2-yl)piperidine-4-carbaldehyde (80 mg, 191.13 μmol, 1 eq.) and (S)-3-(1-oxo-5-(piperazin-1-yl)isoindolin-2-yl)piperidine-2,6-dione (75.31 mg, 229.35 μmol, 1.2 eq.) in DCE (10 mL) was added DIEA (49.40 mg, 382.25 μmol, 66.58 uL, 2 eq.). After addition, the mixture was stirred at 25° C. for 30 min, and then NaBH(OAc) 3 (121.52 mg, 573.38 μmol, 3 eq.) was added. The mixture was stirred at 25° C. for 3 hr. After completion, the reaction mixture was concentrated and purified by preparative HPLC(Column: C18-1 150*30 mm+5 um; Condition water (NH₄HCO₃)-CAN, B %: 58%-88%, 11 min) followed by lyophilization to yield (S)-3-(5-(4-((1-(5-((1R,2S)-2-cyclohexyl-6-hydroxy-1,2,3,4-tetrahydronaphthalen-1-yl)pyridin-2-yl)piperidin-4-yl)methyl)piperazin-1-yl)-1-oxoisoindolin-2-yl)piperidine-2,6-dione (45.4 mg, 62.11 μmol, 32.50% yield, 100% purity) as a white solid. LC-MS (ESI⁺) m/z: 731.6 (M+H)⁺.

LCMS: calc. for C₄₄H₅₄N₆O₄:730.42, found: [M+H]⁺ 731.6.

HPLC: 100% purity at 220 nm.

¹H NMR: ¹H NMR (400 MHZ, METHANOL-d₄) δ ppm 7.77-7.79 (1H, m), 7.62-7.65 (1H, m), 7.12-7.16 (1H, m), 7.06-7.10 (2H, m), 6.69 (2H, dd, J=8.46, 6.79 Hz), 6.54-6.57 (1H, m), 6.46-6.50 (1H, m), 5.10 (1H, dd, J=13.29, 5.07 Hz), 4.40 (2H, d, J=6.08 Hz), 4.10-4.18 (3H, m), 3.35-3.38 (4H, m), 2.86-2.94 (2H, m), 2.81-2.83 (1H, m), 2.77-2.81 (2H, m), 2.59-2.63 (4H, m), 2.43-2.52 (1H, m), 2.27-2.31 (2H, m), 2.12-2.19 (2H, m), 1.89 (4H, br s), 1.74-1.79 (1H, m), 1.60-1.68 (3H, m), 1.46-1.54 (1H, m), 1.38-1.44 (1H, m), 1.20 (4H, br s), 1.12-1.17 (1H, m), 1.01-1.11 (3H, m), 0.81-0.91 (1H, m).

Step 10: To a solution of 1-(5-((1S,2R)-2-cyclohexyl-6-hydroxy-1,2,3,4-tetrahydronaphthalen-1-yl)pyridin-2-yl)piperidine-4-carbaldehyde (80 mg, 191.13 μmol, 1 eq.) and (S)-3-(1-oxo-5-(piperazin-1-yl)isoindolin-2-yl)piperidine-2,6-dione (69.04 mg, 210.24 μmol, 1.1 eq.) in DCE (15 mL) was added DIEA (49.40 mg, 382.25 μmol, 66.58 uL, 2 eq.). After addition, the mixture was stirred at 25° C. for 30 min, and then NaBH(OAc) 3 (81.02 mg, 382.25 μmol, 2 eq.) was added. The mixture was stirred at 25° C. for 3 h. After completion, the reaction mixture was concentrated and purified by preparative HPLC(Column: C18-1 150*30 mm*5 um; Condition water (NH₄HCO₃)-CAN, B %: 58%-88%, 11 min) followed by lyophilization to yield (S)-3-(5-(4-((1-(5-((1S,2R)-2-cyclohexyl-6-hydroxy-1,2,3,4-tetrahydronaphthalen-1-yl)pyridin-2-yl)piperidin-4-yl)methyl)piperazin-1-yl)-1-oxoisoindolin-2-yl)piperidine-2,6-dione (67 mg, 91.66 μmol, 47.96% yield, 100% purity) as a white solid. LC-MS (ESI⁺) m/z 731.6 (M+H)⁺.

LCMS: calc. for C₄₄H₅₄N₆O₄:730.42, found: [M+H]⁺ 731.6.

HPLC: 100% purity at 220 nm.

¹H NMR: ¹H NMR (400 MHZ, METHANOL-d₄) δ ppm 7.78 (1H, d, J=2.27 Hz), 7.63 (1H, d, J=8.58 Hz), 7.14 (1H, dd, J=8.94, 2.38 Hz), 7.05-7.10 (2H, m), 6.69 (2H, dd, J=8.58, 5.84 Hz), 6.56 (1H, d, J=2.26 Hz), 6.46-6.50 (1H, m), 5.10 (1H, br d, J=8.11 Hz), 4.40 (2H, d, J=5.72 Hz), 4.11-4.18 (3H, m), 3.35-3.38 (4H, m), 2.89-2.96 (2H, m), 2.86 (1H, br d, J=5.25 Hz), 2.80 (2H, br dd, J=9.06, 2.62 Hz), 2.59-2.64 (4H, m), 2.43-2.49 (1H, m), 2.29 (2H, br d, J=6.56 Hz), 2.13-2.18 (2H, m), 1.83-1.89 (4H, m), 1.75-1.80 (1H, m), 1.60-1.67 (3H, m), 1.49 (1H, br dd, J=12.58, 5.78 Hz), 1.41 (1H, br d, J=12.99 Hz), 1.25 (4H, br dd, J=11.68, 10.25 Hz), 1.11-1.16 (1H, m), 1.02-1.11 (3H, m), 0.82-0.90 (1H, m).

### Example 255. Preparation of (I-150) (S)-3-(5-(4-((1-(6-((1R,2S)-2-cyclohexyl-6-hydroxy-1,2,3,4-tetrahydronaphthalen-1-yl)pyridin-3-yl)piperidin-4-yl)methyl)piperazin-1-yl)-1-oxoisoindolin-2-yl)piperidine-2,6-dione and (I-149) (S)-3-(5-(4-((1-(6-((1S,2R)-2-cyclohexyl-6-hydroxy-1,2,3,4-tetrahydronaphthalen-1-yl)pyridin-3-yl)piperidin-4-yl)methyl)piperazin-1-yl)-1-oxoisoindolin-2-yl)piperidine-2,6-dione

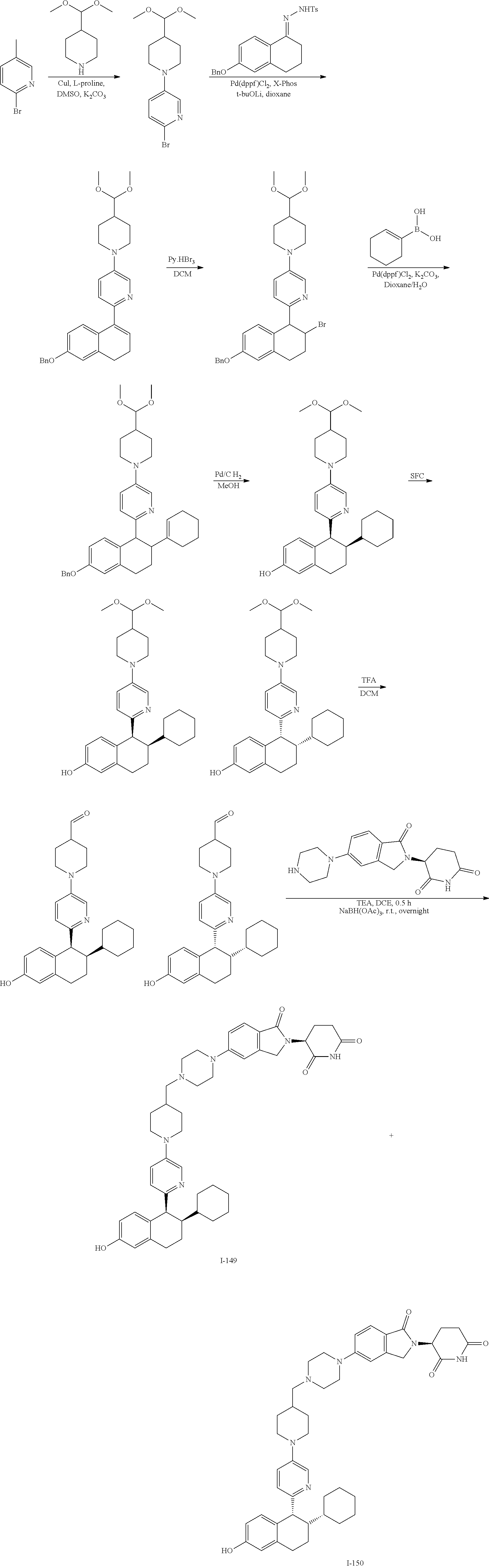

Step 1: A mixture of 2-bromo-5-iodopyridine (5 g, 17.61 mmol, 1 eq.), 4-(dimethoxymethyl)piperidine (3.37 g, 21.13 mmol, 1.2 eq.), CuI (2.01 g, 10.57 mmol, 0.6 eq.), L-proline (1.22 g, 10.57 mmol, 0.6 eq.) and K₂CO₃ (4.87 g, 35.22 mmol, 2 eq.) in DMSO (15 mL) was degassed and purged with N₂, and then the mixture was stirred at 90° C. for 3 hr under N₂ atmosphere. LCMS showed the ˜46% desired MS was detected. The reaction mixture was extracted with EtOAc (40 mL×3) and concentrated to get crude product. The crude product was purified on silica gel column chromatography (from PE/EtOAc=3/1 to 2/1, TLC:

PE/EtOAc=3/1, Rf=0.57) to yield desired 2-bromo-5-(4-(dimethoxymethyl)piperidin-1-yl)pyridine (1.2 g, 90%) as a white solid. LC-MS (ESI⁺) m/z: 316.9 (M+H⁺+2).

Step 2: A mixture of 2-bromo-5-(4-(dimethoxymethyl)piperidin-1-yl)pyridine (1.1 g, 3.49 mmol, 1 eq.), (E)-N′-(6-(benzyloxy)-3,4-dihydronaphthalen-1 (2H)-ylidene)-4-methylbenzenesulfonohydrazide (1.61 g, 3.84 mmol, 1.1 eq.), Pd(dppf)Cl₂·CH₂Cl (284.99 mg, 348.98 μmol, 0.1 eq.), t-BuOLi (838.12 mg, 10.47 mmol, 943.83 uL, 3 eq.) and X-Phos (147.70 mg, 174.49 μmol, 0.05 eq.) in dioxane (20 mL) was degassed and purged with N₂ for 3 times, and then the mixture was stirred at 100° C. for 16 hr under N₂ atmosphere. LCMS showed the desired MS was detected. The reaction mixture was concentrated under reduced pressure to remove dioxane to get the crude product, which was purified by silica gel column chromatography (from PE/EtOAc=3/1 to 2/1, TLC: PE/EtOAc=3/1, Rf=0.51) to yield desired 2-(6-(benzyloxy)-3,4-dihydronaphthalen-1-yl)-5-(4-(dimethoxymethyl)piperidin-1-yl)pyridine (0.5 g, 85%) as yellow solid. LC-MS (ESI⁺) m/z: 471.2 (M+H)⁺.

Step 3: To a solution of 5-(6-(benzyloxy)-3,4-dihydronaphthalen-1-yl)-2-(4-(dimethoxymethyl)piperidin-1-yl)pyridine (500 mg, 1.06 mmol, 1 eq.) in DCM (20 mL) was added Py·HBr₃ (271.84 mg, 849.98 μmol, 0.8 eq.). The mixture was stirred at 25° C. for 0.1 hr. LCMS showed the desired MS was detected. The reaction mixture was concentrated in vacuum, which was extracted with DCM (50 mL×3) and concentrated to get crude product. The crude product was purified by silica gel chromatography (from PE/EtOAc=3/1 to 2/1, TLC: PE/EtOAc=3/1, Rf=0.38) to give 5-(6-(benzyloxy)-2-bromo-3,4-dihydronaphthalen-1-yl)-2-(4-(dimethoxymethyl)piperidin-1-yl)pyridine (0.36 g, 94%) as yellow oil. LC-MS (ESI⁺) m/z: 551.2 (M+H⁺+2).

Step 4: A mixture of 2-(6-(benzyloxy)-2-bromo-3,4-dihydronaphthalen-1-yl)-5-(4-(dimethoxymethyl)piperidin-1-yl)pyridine (350 mg, 636.95 μmol, 1 eq.), cyclohex-1-en-1-ylboronic acid (96.28 mg, 764.33 μmol, 1.2 eq.), Pd(dppf)Cl₂ (46.61 mg, 63.69 μmol, 0.1 eq.) and K₂CO₃ (264.10 mg, 1.91 mmol, 3 eq.) in Dioxane/H2O (15 mL) was degassed and purged with N₂ for 3 times, and then the mixture was stirred at 80° C. for 3 hr under N₂ atmosphere. LCMS showed desired compound was detected. The crude product was purified on silica gel column chromatography (from PE/EtOAc=3/1 to 2/1, TLC: PE/EtOAc=2/1, Rf=0.4) to yield 2-(6-(benzyloxy)-2-(cyclohex-1-en-1-yl)-3,4-dihydronaphthalen-1-yl)-5-(4-(dimethoxymethyl)piperidin-1-yl)pyridine (0.19 g, 84%) as yellow oil. LC-MS (ESI⁺) m/z: 551.3 (M+H)⁺.

Step 5: To a solution of 2-(6-(benzyloxy)-2-(cyclohex-1-en-1-yl)-3,4-dihydronaphthalen-1-yl)-5-(4-(dimethoxymethyl)piperidin-1-yl)pyridine (250.00 mg, 453.94 μmol, 1 eq.) in MeOH (15 mL) was added Pd/C (200 mg, 1.18 mmol, 2.59 eq.) under H₂ atmosphere. The suspension was degassed and purged with H₂ (32.96 mg, 16.32 mmol, 35.95 eq.) for 3 times. The mixture was stirred under H₂ (50 psi) at 50° C. for 12 hr. LCMS showed desired compound was detected. The mixture was filtered and the filtrate was concentrated to yield 6-cyclohexyl-5-(5-(4-(dimethoxymethyl)piperidin-1-yl)pyridin-2-yl)-5,6,7,8-tetrahydronaphthalen-2-ol (0.12 g, 84%) as yellow solid used for the next step directly. LC-MS (ESI⁺) m/z: 465.1 (M+H)⁺.

Step 6: 6-cyclohexyl-5-(5-(4-(dimethoxymethyl)piperidin-1-yl)pyridin-2-yl)-5,6,7,8-tetrahydronaphthalen-2-ol (120.00 mg, 258.27 μmol, 1 eq.) was separated by chiral SFC (column: DAICEL CHIRALPAK AD 250 mm*30 mm, 10 um); mobile phase: [CO₂-ACN/MeOH (0.1% NH₃H₂O)]; B %: 45%-45%; Flow Rate: 80 ml/min; peak 1:1.730 min; peak 2:2.256 min) to yield peak 1 and peak 2.

Peak 1 was concentrated under reduced pressure to yield (5R,6S)-6-cyclohexyl-5-(5-(4-(dimethoxymethyl)piperidin-1-yl)pyridin-2-yl)-5,6,7,8-tetrahydronaphthalen-2-ol (40 mg, 33.33% yield) as yellow oil. LC-MS (ESI⁺) m/z: 465.3 (M+H)⁺,

Peak 2 was concentrated under reduced pressure to yield (5S,6R)-6-cyclohexyl-5-(5-(4-(dimethoxymethyl)piperidin-1-yl)pyridin-2-yl)-5,6,7,8-tetrahydronaphthalen-2-ol (20 mg, 16.67% yield) as yellow oil. LC-MS (ESI⁺) m/z: 465.3 (M+H)⁺.

Step 7: To a solution of (5R,6S)-6-cyclohexyl-5-(5-(4-(dimethoxymethyl)piperidin-1-yl)pyridin-2-yl)-5,6,7,8-tetrahydronaphthalen-2-ol (40.00 mg, 86.09 μmol, 1 eq.) in DCM (6 mL) was added TFA (2.05 g, 17.95 mmol, 1.33 mL, 208.51 eq.). The mixture was stirred at 25° C. for 12 hr. LCMS showed desired compound was detected. After completion, the mixture was concentrated under reduced pressure to give 1-(6-((1R,2S)-2-cyclohexyl-6-hydroxy-1,2,3,4-tetrahydronaphthalen-1-yl)pyridin-3-yl)piperidine-4-carbaldehyde (40 mg, crude) as yellow oil without purification. LC-MS (ESI⁺) m/z: 419.3 [M+H]⁺

Step 8: To a solution of (5S,6R)-6-cyclohexyl-5-(5-(4-(dimethoxymethyl)piperidin-1-yl)pyridin-2-yl)-5,6,7,8-tetrahydronaphthalen-2-ol (20.00 mg, 43.04 μmol, 1 eq.) in DCM (6 mL) was added TFA (1.02 g, 8.98 mmol, 1.33 mL, 208.51 eq.). The mixture was stirred at 25° C. for 12 hr. LCMS showed desired compound was detected. After completion, the mixture was concentrated under reduced pressure to give 1-(6-((1S,2R)-2-cyclohexyl-6-hydroxy-1,2,3,4-tetrahydronaphthalen-1-yl)pyridin-3-yl)piperidine-4-carbaldehyde (40 mg, crude) as yellow oil without purification. LC-MS (ESI⁺) m/z: 419.3 [M+H]⁺

Step 9: To a solution of 1-(6-((1R,2S)-2-cyclohexyl-6-hydroxy-1,2,3,4-tetrahydronaphthalen-1-yl)pyridin-3-yl)piperidine-4-carbaldehyde (40 mg, 95.56 μmol, 1 eq.) and (S)-3-(1-oxo-5-(piperazin-1-yl)isoindolin-2-yl)piperidine-2,6-dione (34.52 mg, 105.12 μmol, 1.1 eq.) in DCE (10 mL) was added TEA (19.34 mg, 191.13 μmol, 26.60 μL, 2 eq.). After addition, the mixture was stirred at 25° C. for 30 min, and then NaBH(OAc) 3 (40.51 mg, 191.13 μmol, 2 eq.) was added. The mixture was stirred at 25° C. for 3 hr. After completion, the reaction mixture was concentrated and purified by preparative HPLC(Column: Boston Green ODS 150*30 mm*5 um; Condition water (NH₄HCO₃)-CAN, B %: 52%-82%, 11 min) followed by lyophilization to yield (S)-3-(5-(4-((1-(6-((1R,2S)-2-cyclohexyl-6-hydroxy-1,2,3,4-tetrahydronaphthalen-1-yl)pyridin-3-yl)piperidin-4-yl)methyl)piperazin-1-yl)-1-oxoisoindolin-2-yl)piperidine-2,6-dione (4.6 mg, 6.21 μmol, 6.50% yield, 98.71% purity) as a white solid.

LC-MS (ESI⁺) m/z: 731.6 [M+H]⁺.

LCMS: calc. for C₄₄H₅₄N₆O₄:730.42, found: [M+H]⁺ 731.6.

HPLC: 98.71% purity at 220 nm.

¹H NMR: ¹H NMR (400 MHZ, METHANOL-d₄) δ ppm 8.08 (1H, s), 7.62-7.70 (1H, m), 7.25-7.31 (1H, m), 7.08-7.13 (2H, m), 6.82-6.87 (1H, m), 6.66-6.71 (1H, m), 6.57-6.62 (1H, m), 6.44-6.51 (1H, m), 5.12 (1H, br s), 4.42 (2H, br d, J=6.08 Hz), 4.29 (1H, br s), 3.70-3.74 (2H, m), 3.39 (4H, br s), 2.93-2.99 (2H, m), 2.76-2.82 (3H, m), 2.62-2.67 (4H, m), 2.46-2.51 (1H, m), 2.33 (2H, br d, J=6.91 Hz), 2.16-2.21 (1H, m), 2.07-2.11 (1H, m), 1.94 (2H, br d, J=12.99 Hz), 1.76-1.83 (4H, m), 1.72 (1H, br dd, J=7.81, 1.85 Hz), 1.56-1.62 (2H, m), 1.33-1.42 (3H, m), 1.13-1.23 (3H, m), 1.10 (1H, br dd, J=6.91, 2.38 Hz), 0.98-1.04 (2H, m), 0.74-0.87 (1H, m).

Step 10: To a solution of 1-(6-((1S,2R)-2-cyclohexyl-6-hydroxy-1,2,3,4-tetrahydronaphthalen-1-yl)pyridin-3-yl)piperidine-4-carbaldehyde (40 mg, 95.56 μmol, 1 eq.) and (S)-3-(1-oxo-5-(piperazin-1-yl)isoindolin-2-yl)piperidine-2,6-dione (34.52 mg, 105.12 μmol, 1.1 eq.) in DCE (10 mL) was added TEA (19.34 mg, 191.13 μmol, 26.60 μL, 2 eq.). After addition, the mixture was stirred at 25° C. for 30 min, and then NaBH(OAc)₃ (40.51 mg, 191.13 μmol, 2 eq.) was added. The mixture was stirred at 25° C. for 3 hr. After completion, the reaction mixture was concentrated and purified by preparative HPLC(Column: Boston Green ODS 150*30 mm*5 um; Condition water (NH₄HCO₃)-CAN, B %: 52%-82%, 11 min) followed by lyophilization to yield (S)-3-(5-(4-((1-(6-((1S,2R)-2-cyclohexyl-6-hydroxy-1,2,3,4-tetrahydronaphthalen-1-yl)pyridin-3-yl)piperidin-4-yl)methyl)piperazin-1-yl)-1-oxoisoindolin-2-yl)piperidine-2,6-dione (9.1 mg, 12.45 μmol, 13.03% yield, 100% purity) as a white solid.

LC-MS (ESI⁺) m/z 731.6 [M+H]⁺.

LCMS: calc. for C₄₄H₅₄N₆O₄:730.42, found: [M+H]⁺ 731.6.

HPLC: 100% purity at 220 nm.

¹H NMR: ¹H NMR (400 MHZ, METHANOL-d₄) δ ppm 8.10 (1H, d, J=2.74 Hz), 7.66 (1H, d, J=8.46 Hz), 7.24-7.30 (1H, m), 7.07-7.13 (2H, m), 6.81-6.86 (1H, m), 6.69 (1H, d, J=8.23 Hz), 6.57-6.61 (1H, m), 6.45-6.50 (1H, m), 5.08-5.15 (1H, m), 4.36-4.47 (2H, m), 4.29 (1H, br d, J=3.34 Hz), 3.68-3.77 (2H, m), 3.37-3.41 (4H, m), 2.88-3.01 (2H, m), 2.80-2.83 (1H, m), 2.74-2.79 (2H, m), 2.60-2.66 (4H, m), 2.42-2.54 (1H, m), 2.30-2.35 (2H, m), 2.14-2.20 (1H, m), 2.08 (1H, br d, J=12.16 Hz), 1.91-1.97 (2H, m), 1.83 (4H, br s), 1.74 (1H, br s), 1.54-1.63 (2H, m), 1.30-1.43 (3H, m), 1.10-1.24 (3H, m), 1.04-1.09 (1H, m), 0.97-1.04 (2H, m), 0.75-0.85 (1H, m).

### Example 256. Preparation of (I-5) (35)-3-[5-[4-[[1-[4-[(1S,2S)-2-cyclohexyl-6-hydroxy-tetralin-1-yl]phenyl]-4-piperidyl]methyl]piperazin-1-yl]-1-oxo-isoindolin-2-yl]piperidine-2,6-dione and (I-6) (35)-3-[5-[4-[[1-[4-[(1R,2R)-2-cyclohexyl-6-hydroxy-tetralin-1-yl]phenyl]-4-piperidyl]methyl]piperazin-1-yl]-1-oxo-isoindolin-2-yl]piperidine-2,6-dione

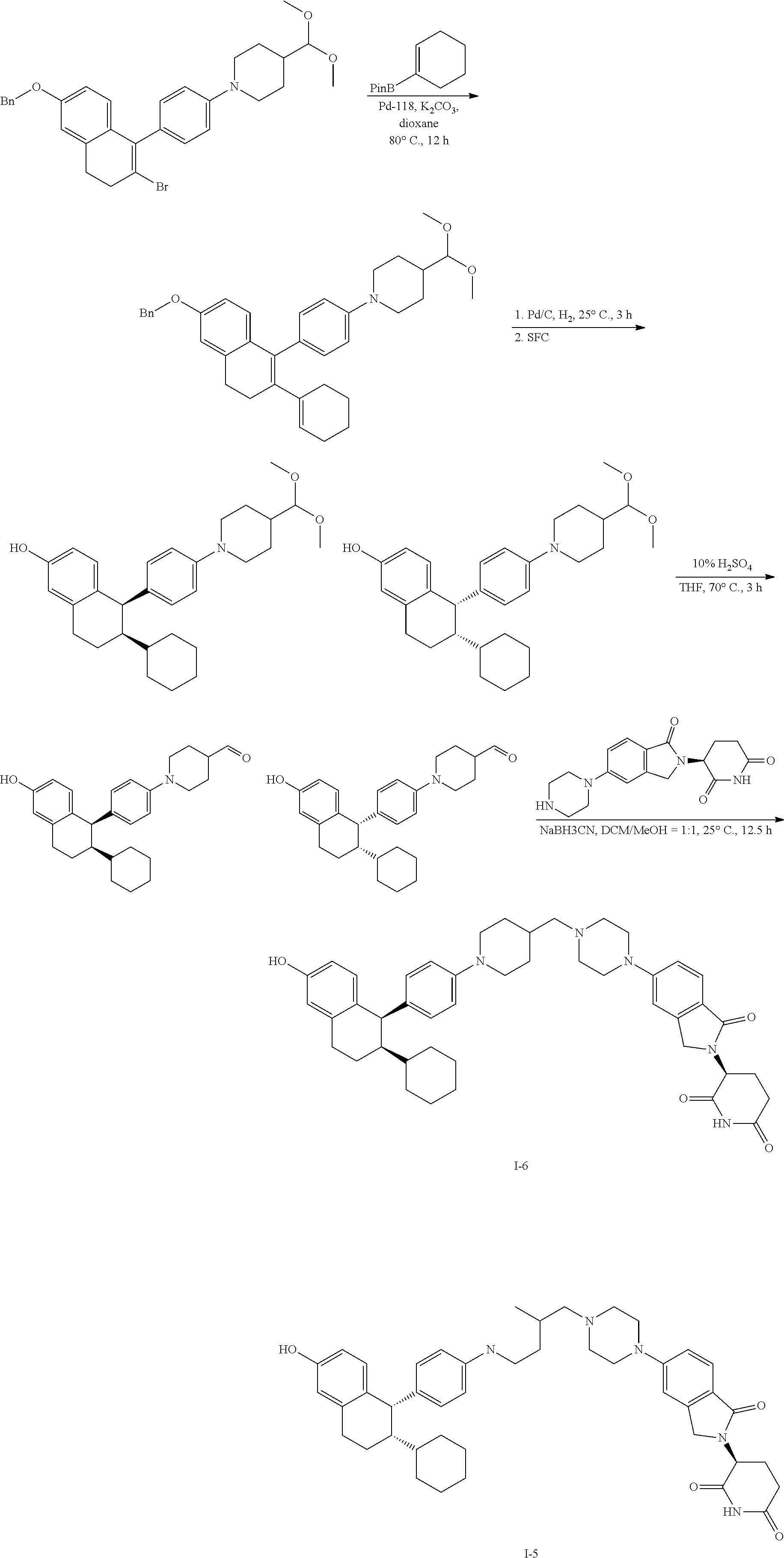

Step 1: A mixture of 1-(4-(6-(benzyloxy)-2-bromo-3,4-dihydronaphthalen-1-yl)phenyl)-4-(dimethoxymethyl)piperidine (800 mg, 1.46 mmol), 2-(cyclohex-1-en-1-yl)-4,4,5,5-tetramethyl-1,3,2-dioxaborolane (334 mg, 1.60 mmol), 1,1′-Bis(di-t-butylphosphino)ferrocene palladium dichloride (95.1 mg, 146 μmol) and Na₂CO₃ (309 mg, 2.92 mmol) in 1,4-dioxane (10 mL) and H₂O (2 mL) was degassed and purged with N₂ for 3 times, and then the mixture was stirred at 80° C. for 12 h under N₂ atmosphere. The mixture was diluted with water (20 mL) and extracted with ethyl acetate (30 mL×3). The combined organic phases were washed with brine (30 mL), dried over anhydrous Na₂SO₄, filtered and concentrated under reduced pressure to give a residue. The residue was purified by flash silica gel chromatography (ethyl acetate in petroleum ether=0% to 25%) to give the product of 1-(4-(6-(benzyloxy)-2-(cyclohex-1-en-1-yl)-3,4-dihydronaphthalen-1-yl)phenyl)-4-(dimethoxymethyl)piperidine (450 mg, 53.0% yield) as a yellow solid. LC-MS (ESI⁺) m/z: 550.5 (M+H)⁺.

Step 2: To a solution of Pd/C (400 mg, 10.0% purity, 376 μmol) in MeOH (15 mL) was added methyl 1-(4-(6-(benzyloxy)-2-(cyclohex-1-en-1-yl)-3,4-dihydronaphthalen-1-yl)phenyl)-4-(dimethoxymethyl)piperidine (450 mg, 794 μmol) under N₂. The suspension was degassed under vacuum and purged with H₂ several times. The mixture was stirred under H₂ (15 psi) at 25° C. for 3 h. The reaction mixture was filtered and the filter was concentrated to give a residue which was separated by chiral SFC (column: DAICEL CHIRALCEL OJ (250 mm*30 mm, 10 um); mobile phase: [0.1% NH₃H₂O EtOH]; B %: 30%-30%, FlowRate (80 ml/min) peak 1:1.835 min, peak 2: 2.088 min) to yield Peak 1 and Peak 2. Peak 1 was concentrated under reduced pressure to yield (5S,6S)-6-cyclohexyl-5-(4-(4-(dimethoxymethyl)piperidin-1-yl)phenyl)-5,6,7,8-tetrahydronaphthalen-2-ol (120 mg, 29.9% yield) as a yellow solid. LC-MS (ESI⁺) m/z: 464.4 (M+H)⁺.

Peak 2 was concentrated under reduced pressure to yield (5R,6R)-6-cyclohexyl-5-(4-(4-(dimethoxymethyl)piperidin-1-yl)phenyl)-5,6,7,8-tetrahydronaphthalen-2-ol (120 mg, 29.9% yield) as a yellow solid. LC-MS (ESI⁺) m/z: 464.4 (M+H)⁺.

Step 3: To a solution of (1S,2S)-2-cyclohexyl-1-[4-[4-(dimethoxymethyl)-1-piperidyl]phenyl]tetralin-6-ol (120 mg, 258.82 μmol) in THF (3 mL) was added H₂SO₄ (3 mL, 5.63 mmol, 10.0% purity). The mixture was stirred at 70° C. for 3 h. The reaction mixture was quenched by addition of water (10 mL), adjust pH to 8 by NaHCO₃ saturated solution, extracted with EtOAc (20 mL×3). The combined organic layers were washed with brine (10 mL), dried over Na₂SO₄, filtered and concentrated under reduced pressure to give 1-[4-[(1R,2R)-2-cyclohexyl-6-hydroxy-tetralin-1-yl]phenyl]piperidine-4-carbaldehyde (130 mg, 96.23% yield) as a yellow solid. LC-MS (ESI⁺) m/z: 418.4 (M+H)⁺.

Step 4: To a solution of (1R,2R)-2-cyclohexyl-1-[4-[4-(dimethoxymethyl)-1-piperidyl]phenyl]tetralin-6-ol (120 mg, 258.82 μmol) in THF (3 mL) was added H₂SO₄ (3 mL, 5.63 mmol, 10.0% purity). The mixture was stirred at 70° C. for 3 h. The reaction mixture was quenched by addition of water (10 mL), adjust pH to 8 by NaHCO₃ saturated solution, extracted with EtOAc (20 mL×3). The combined organic layers were washed with brine (10 mL), dried over Na₂SO₄, filtered and concentrated under reduced pressure to give 1-[4-[(1R,2R)-2-cyclohexyl-6-hydroxy-tetralin-1-yl]phenyl]piperidine-4-carbaldehyde (130 mg, 96.2% yield) as a yellow solid. LC-MS (ESI⁺) m/z: 418.4 (M+H)⁺.

Step 5: To a solution of 1-[4-[(1S,2S)-2-cyclohexyl-6-hydroxy-tetralin-1-yl]phenyl]piperidine-4-carbaldehyde (60 mg, 114.95 μmol) and (35)-3-(1-oxo-5-piperazin-1-yl-isoindolin-2-yl)piperidine-2,6-dione (37.74 mg, 114.95 μmol) in DCM (2.5 mL) and MeOH (2.5 mL) after stirred 30 min was added sodium;cyanoboranuide (36.12 mg, 574.74 μmol). The mixture was stirred at 25° C. for 12.5 h. The reaction mixture was concentrated to yield a residue which was purified by preparative HPLC (column: Boston Green ODS 150*30 mm*5 um; mobile phase: [water(FA)-ACN]; B %: 27%-47%, 12 min), followed by lyophilization to give (35)-3-15-[4-[[1-[4-[(1S,2S)-2-cyclohexyl-6-hydroxy-tetralin-1-yl]phenyl]-4-piperidyl]methyl]piperazin-1-yl]-1-oxo-isoindolin-2-yl]piperidine-2,6-dione (30 mg, 35.0% yield, 0.1FA) as a white solid. LC-MS (ESI⁺) m/z: 730.4 (M+H)⁺

LCMS: calc. for C₄₅H₅₅N₅O₄:729.4, found: [M+H]⁺ 730.4.

HPLC: 100% purity at 254 nm.

¹H NMR (500 MHz, MeOD-d4): $8.10 (s, 1H), 7.70 (d, J=9.0 Hz, 1H), 7.67 (s, 1H), 7.21-7.14 (m, 2H), 6.98-6.87 (m, 4H), 6.65 (d, J=8.4 Hz, 1H), 6.55 (d, J=2.1 Hz, 1H), 6.45 (dd, J=2.4, 8.3 Hz, 1H), 5.12 (dd, J=5.2, 13.4 Hz, 1H), 4.50-4.36 (m, 2H), 4.14 (d, J=4.4 Hz, 1H), 3.81-3.51 (m, 4H), 3.41 (s, 5H), 3.10 (d, J=6.4 Hz, 2H), 2.95-2.86 (m, 2H), 2.84-2.70 (m, 4H), 2.47 (d, J=4.5, 13.1 Hz, 1H), 2.18-2.09 (m, 2H), 2.05 (s, 1H), 1.92 (d, J=12.2 Hz, 2H), 1.84-1.72 (m, 2H), 1.67-1.55 (m, 4H), 1.55-1.45 (m, 2H), 1.34-0.96 (m, 7H), 0.85-0.73 (m, 1H).

Step 6: To a solution of 1-[4-[(1R,2R)-2-cyclohexyl-6-hydroxy-tetralin-1-yl]phenyl]piperidine-4-carbaldehyde (60 mg, 114.95 μmol) and (3S)-3-(1-oxo-5-piperazin-1-yl-isoindolin-2-yl)piperidine-2,6-dione (37.74 mg, 114.95 μmol) in DCM (2.5 mL) and MeOH (2.5 mL) after stirred 30 min was added sodium;cyanoboranuide (36.12 mg, 574.74 μmol). The mixture was stirred at 25° C. for 12.5 h. The reaction mixture was concentrated to yield a residue which was purified by preparative HPLC (column: Boston Green ODS 150*30 mm*5 um; mobile phase: [water(FA)-ACN]; B %: 27%-47%, 12 min), followed by lyophilization to give (35)-3-[5-[4-[ ]-[4-[(1R,2R)-2-cyclohexyl-6-hydroxy-tetralin-1-yl]phenyl]-4-piperidyl]methyl]piperazin-1-yl]-1-oxo-isoindolin-2-yl]piperidine-2,6-dione (30.6 mg, 36.3% yield, 0.06FA) as a white solid. LC-MS (ESI⁺) m/z: 730.4 (M+H)⁺

LCMS: calc. for C₄₅H₅₅N₅O₄: 729.4, found: [M+H]⁺ 730.4.

HPLC: 100% purity at 254 nm.

¹H NMR (500 MHz, MeOD-d4): δ 8.09 (s, 1H), 7.73-7.66 (m, 1H), 7.22-7.15 (m, 2H), 6.91 (q, J=8.6 Hz, 4H), 6.65 (d, J=8.4 Hz, 1H), 6.55 (d, J=2.1 Hz, 1H), 6.45 (dd, J=2.4, 8.3 Hz, 1H), 5.12 (dd, J=5.1, 13.4 Hz, 1H), 4.51-4.36 (m, 2H), 4.14 (d, J=4.3 Hz, 1H), 3.68 (s, 4H), 3.45 (s, 5H), 3.17-3.08 (m, 2H), 2.95-2.85 (m, 2H), 2.82-2.70 (m, 4H), 2.47 (d, J=4.5, 13.2 Hz, 1H), 2.27-2.11 (m, 2H), 2.05 (d, J=11.3 Hz, 1H), 1.92 (d, J=12.5 Hz, 2H), 1.81-1.72 (m, 2H), 1.71-1.37 (m, 7H), 1.35-0.98 (m, 7H), 0.79 (q, J=10.9 Hz, 1H).

### Example 257. Preparation of (I-407) (35)-3-[5-[4-[[1-[4-[(1R,2S)-2-(cyclohexen-1-yl)-6-hydroxy-tetralin-1-yl]phenyl]-4-piperidyl]methyl]piperazin-1-yl]-1-oxo-isoindolin-2-yl]piperidine-2,6-dione and (I-416) (35)-3-[5-[4-[[1-[4-[(1S,2R)-2-(cyclohexen-1-yl)-6-hydroxy-tetralin-1-yl]phenyl]-4-piperidyl]methyl]piperazin-1-yl]-1-oxo-isoindolin-2-yl]piperidine-2,6-dione

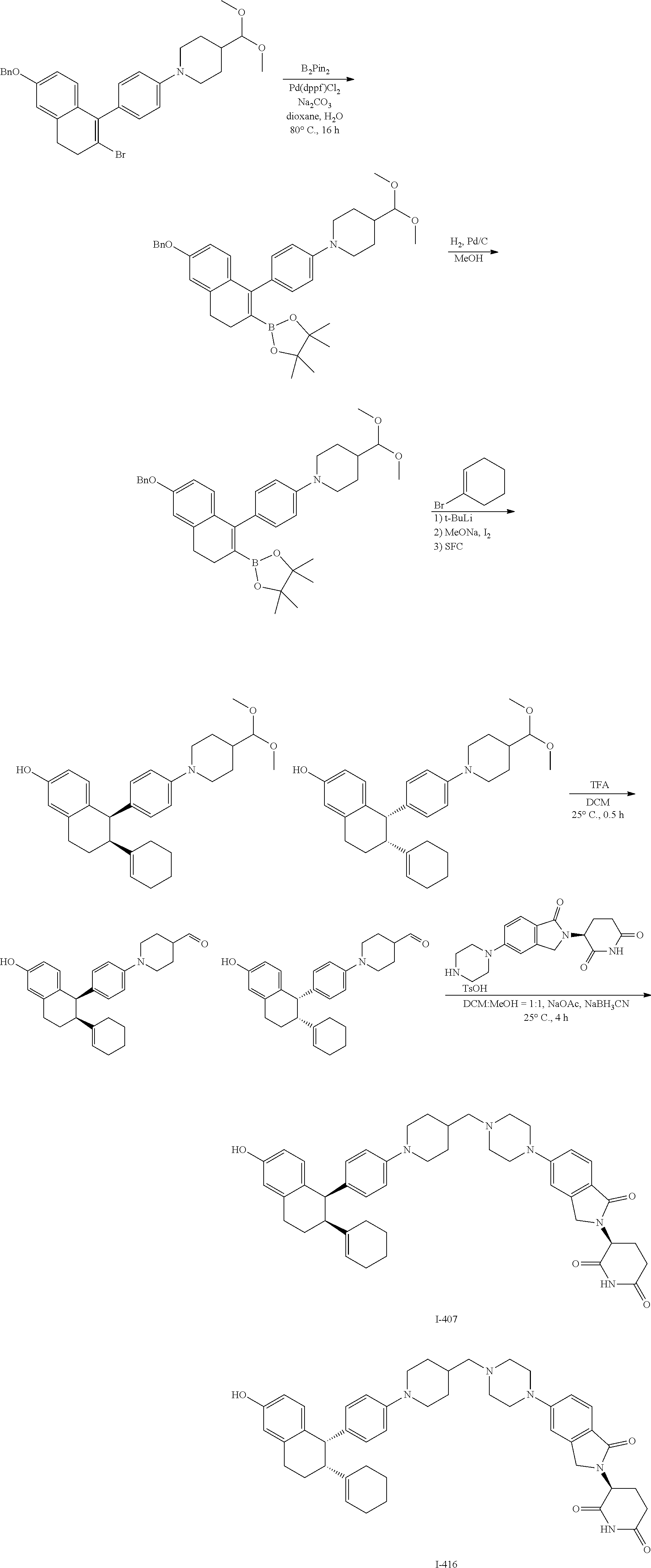

Step 1: A mixture of 1-[4-(6-benzyloxy-2-bromo-3,4-dihydronaphthalen-1-yl)phenyl]-4-(dimethoxymethyl)piperidine (2.8 g, 4.34 mmol), 4,4,5,5-tetramethyl-2-(4,4,5,5-tetramethyl-1,3,2-dioxaborolan-2-yl)-1,3,2-dioxaborolane (1.54 g, 6.07 mmol), Pd(dppf)Cl₂ (317.49 mg, 433.90 μmol), Na₂CO₃ (919.78 mg, 8.68 mmol) in dioxane (30 mL) and H₂O (6 mL) was degassed and purged with N₂ for 3 times, and then the mixture was stirred at 90° C. for 16 h under N₂ atmosphere. The reaction mixture was quenched with water (200 mL) and extracted with EtOAc (300 mL×3). The combined organic layers were washed with brine (300 mL), dried over Na₂SO₄, filtered and concentrated under reduced pressure to give a residue. The residue was purified by flash silica gel chromatography (ethyl acetate in petroleum ether=0% to 20%) to give 1-[4-[6-benzyloxy-2-(4,4,5,5-tetramethyl-1,3,2-dioxaborolan-2-yl)-3,4-dihydronaphthalen-1-yl]phenyl]-4-(dimethoxymethyl)piperidine (2.2 g, 83.4% yield) as a yellow solid. LC-MS (ESI⁺) m/z: 596.1 (M+H)⁺.

Step 2: To a solution of Pd/C (300 mg, 10% purity) in MeOH (10 mL) was added Jan. 14, 2016-benzyloxy-2-(4,4,5,5-tetramethyl-1,3,2-dioxaborolan-2-yl)-3,4-dihydronaphthalen-1-yl]phenyl]-4-(dimethoxymethyl)piperidine (2.2 g, 3.62 mmol) under N₂. The suspension was degassed under vacuum and purged with H₂ several times. The mixture was stirred under H₂ (15 psi) at 25° C. for 16 h. The reaction mixture was filtered and the filter was concentrated. The residue was purified by preparative HPLC (column: Phenomenex C18 150*40 mm*5 um; mobile phase: [water (NH₃H₂O+NH₄HCO₃)-CAN]; B %: 50%-70%, 10 min), followed by lyophilization to give 1-[4-[4-(dimethoxymethyl)-1-piperidyl]phenyl]-2-(4,4,5,5-tetramethyl-1,3,2-dioxaborolan-2-yl)tetralin-6-ol (700 mg, 33.5% yield) as a white solid. LC-MS (ESI⁺) m/z: 508.3 (M+H)⁺.

Step 3: A stirred solution of 1-bromocyclohexene (55.85 mg, 346.82 μmol) in THF (1 mL) was cooled to −78° C. and t-BuLi (1.3 M, 533.57 μL) was added dropwise. The resulting solution was stirred at −78° C. for 30 min and then a solution of 1-[4-[4-(dimethoxymethyl)-1-piperidyl]phenyl]-2-(4,4,5,5-tetramethyl-1,3,2-dioxaborolan-2-yl)tetralin-6-ol (100 mg, 173.41 μmol) in THF (1 mL) was added dropwise. The resulting solution was stirred at −78° C. for 15 min and then warmed to 0° C. and stirred for a further 15 min. After this time, the solution was cooled to −78° C. and a suspension of NaOMe (3 M, 1.16 mL) was added in a single portion followed by dropwise addition of a solution of I₂ (0.5 M, 416.18 uL). The resulting mixture was warmed to 0° C. and stirred for 30 min and then warmed to 25° C. and stirred at 25° C. for 16 h. The reaction was quenched with sat.aq. Na₂O₃S₂ (10 mL) and diluted with water (20 mL) and extracted with DCM (30 mL×3). The combined organic layers were washed with brine (40 mL), dried over Na₂SO₄, filtered and concentrated under reduced pressure to give a residue. The residue was purified by preparative HPLC (column: Phenomenex C18 150*40 mm*5 um; mobile phase: [water (NH₃H₂O+NH₄HCO₃)-CAN]; B %: 60%-90%, 10 min.), followed by lyophilization to yield a product which was separated by chiral SFC (column: DAICEL CHIRALPAK IG (250 mm*30 mm, 10 um); mobile phase: [0.1% NH₃H₂O ETOH]; B %: 40%-40%, min, peak 1: 2.039 min, peak 2:2.291 min) to yield Peak 1 and Peak 2. Peak 1 was concentrated under reduced pressure to yield (1R,2S)-2-(cyclohexen-1-yl)-1-[4-[4-(dimethoxymethyl)-1-piperidyl]phenyl]tetralin-6-ol (40 mg, 9.7% yield) (SFC: Rt=2.039) s a white solid. LC-MS (ESI⁺) m/z: 462.4 (M+H)⁺. Peak 2 was concentrated under reduced pressure to yield (1S,2R)-2-(cyclohexen-1-yl)-1-[4-[4-(dimethoxymethyl)-1-piperidyl]phenyl]tetralin-6-ol (40 mg, 9.79% yield) (SFC: Rt=2.291) as a white solid. LC-MS (ESI⁺) m/z: 462.4 (M+H)⁺.

Step 4: To a solution of (1R,2S)-2-(cyclohexen-1-yl)-1-[4-[4-(dimethoxymethyl)-1-piperidyl]phenyl]tetralin-6-ol (40 mg, 84.05 μmol) in DCM (3 mL) was added TFA (9.58 mg, 84.05 μmol, 6.22 uL). The mixture was stirred at 25° C. for 10 min. The solvent was removed to yield a residue which was added H₂O (3 mL) and CAN (5 mL), followed by lyophilization to give 1-[4-[(1R,2S)-2-(cyclohexen-1-yl)-6-hydroxy-tetralin-1-yl]phenyl]piperidine-4-carbaldehyde (30 mg, crude) as a brown solid. LC-MS (ESI⁺) m/z: 416.2 (M+H)⁺,

Step 5: To a solution of (1S,2R)-2-(cyclohexen-1-yl)-1-[4-[4-(dimethoxymethyl)-1-piperidyl]phenyl]tetralin-6-ol (40 mg, 84.92 μmol) in DCM (3 mL) was added TFA (9.68 mg, 84.92 μmol, 6.29 μL). The mixture was stirred at 25° C. for 10 min. The solvent was removed to yield a residue which was added H₂O (3 mL) and ACN (5 mL), followed by lyophilization to give 1-[4-[(1S,2R)-2-(cyclohexen-1-yl)-6-hydroxy-tetralin-1-yl]phenyl]piperidine-4-carbaldehyde (30 mg, crude) as a brown solid. LC-MS (ESI⁺) m/z: 416.2 (M+H)⁺.

Step 6: To a solution of 1-[4-[(1R,2S)-2-(cyclohexen-1-yl)-6-hydroxy-tetralin-1-yl]phenyl]piperidine-4-carbaldehyde (40 mg, 96.25 μmol) in MeOH (3 mL) and DCM (3 mL) was added NaOAc (15.79 mg, 192.51 μmol), and (35)-3-(1-oxo-5-piperazin-1-yl-isoindolin-2-yl)piperidine-2,6-dione (48.18 mg, 96.25 μmol, TsOH) and stirred at 25° C. for 1 h. Then NaBH₃CN (30.24 mg, 481.27 μmol) was added and stirred at 25° C. for 3 h. The solvent was removed to yield a residue. The residue was purified by preparative HPLC (column: Boston Green ODS 150*30 mm*5 um; mobile phase: [water(FA)-ACN]; B %: 16%-46%, 10 min), followed by lyophilization to yield (3S)-3-[5-[4-[[1-[4-[(1R,2S)-2-(cyclohexen-1-yl)-6-hydroxy-tetralin-1-yl]phenyl]-4-piperidyl]methyl]piperazin-1-yl]-1-oxo-isoindolin-2-yl]piperidine-2,6-dione (13.0 mg, 18.2% yield, 0.3FA) as a white solid. LC-MS (ESI⁺) m/z: 728.6 (M+H)⁺

LCMS: calc. for C₄₅H₅₃N₅O₄: 727.9, found: [M+H]⁺ 728.6.

HPLC: 100.00% purity at 254 nm.

¹HNMR (400 MHz, MeOD-d4): δ 8.40 (s, 1H), 7.65 (d, J=8.7 Hz, 1H), 7.17-7.05 (m, 2H), 6.85-6.80 (m, 2H), 6.76-6.71 (m, 2H), 6.68 (d, J=8.3 Hz, 1H), 6.57 (d, J=2.4 Hz, 1H), 6.48 (dd, J=2.5, 8.3 Hz, 1H), 5.10 (dd,]=5.1, 13.4 Hz, 1H), 4.97 (s, 1H), 4.46-4.34 (m, 2H), 4.09 (d, J=5.0 Hz, 1H), 3.60 (d, J=9.1 Hz, 2H), 3.47-3.37 (m, 4H), 2.96-2.72 (m, 8H), 2.65 (brt, J=12.0 Hz, 2H), 2.54-2.39 (m, 4H), 2.20-2.06 (m, 2H), 2.00-1.75 (m, 6H), 1.58-1.34 (m, 8H).

Step 7: To a solution of 1-[4-[(1S,2R)-2-(cyclohexen-1-yl)-6-hydroxy-tetralin-1-yl]phenyl]piperidine-4-carbaldehyde (40 mg, 96.25 μmol) in MeOH (3 mL) and DCM (3 mL) was added NaOAc (15.79 mg, 192.51 μmol) and (35)-3-(1-oxo-5-piperazin-1-yl-isoindolin-2-yl)piperidine-2,6-dione (48.18 mg, 96.25 μmol, TsOH) and stirred at 25° C. for 1 h. Then NaBH₃CN (30.24 mg, 481.27 μmol) was added and stirred at 25° C. for 3 h. The solvent was removed to yield a residue. The residue was purified by preparative HPLC (column: Boston Green ODS 150*30 mm*5 um; mobile phase: [water(FA)-CAN]; B %: 16%-46%, 10 min), followed by lyophilization to yield (3S)-3-[5-[4-[[1-[4-[(1S,2R)-2-(cyclohexen-1-yl)-6-hydroxy-tetralin-1-yl]phenyl]-4-piperidyl]methyl]piperazin-1-yl]-1-oxo-isoindolin-2-yl]piperidine-2,6-dione (14.3 mg, 19.9% yield, 0.35FA) as a white solid. LC-MS (ESI⁺) m/z: 728.6 (M+H)⁺

LCMS: calc. for C₄₅H₅₃N₅O₄:727.9, found: [M+H]⁺ 728.6.

HPLC: 100.00% purity at 254 nm.

¹HNMR (400 MHz, MeOD-d4): δ 8.38 (s, 1H), 7.65 (d, J=8.7 Hz, 1H), 7.15-7.04 (m, 2H), 6.86-6.80 (m, 2H), 6.76-6.72 (m, 2H), 6.68 (d, J=8.3 Hz, 1H), 6.57 (d, J=2.5 Hz, 1H), 6.48 (dd, J=2.6, 8.3 Hz, 1H), 5.10 (dd, J=5.2, 13.3 Hz, 1H), 4.97 (s, 1H), 4.47-4.33 (m, 2H), 4.09 (d, J=5.0 Hz, 1H), 3.60 (d, J=8.9 Hz, 2H), 3.47-3.37 (m, 4H), 2.96-2.73 (m, 8H), 2.66 (t, J=12.0 Hz, 2H), 2.54-2.38 (m, 4H), 2.20-2.08 (m, 2H), 1.99-1.72 (m, 6H), 1.58-1.33 (m, 8H).

### Example 258. Preparation of (I-32) (3S)-3-15-[4-[[1-[4-[(1R,2R)-6-hydroxy-2-tetrahydropyran-4-yl-tetralin-1-yl]phenyl]-4-piperidyl]methyl]piperazin-1-yl]-1-oxo-isoindolin-2-yl]piperidine-2,6-dione

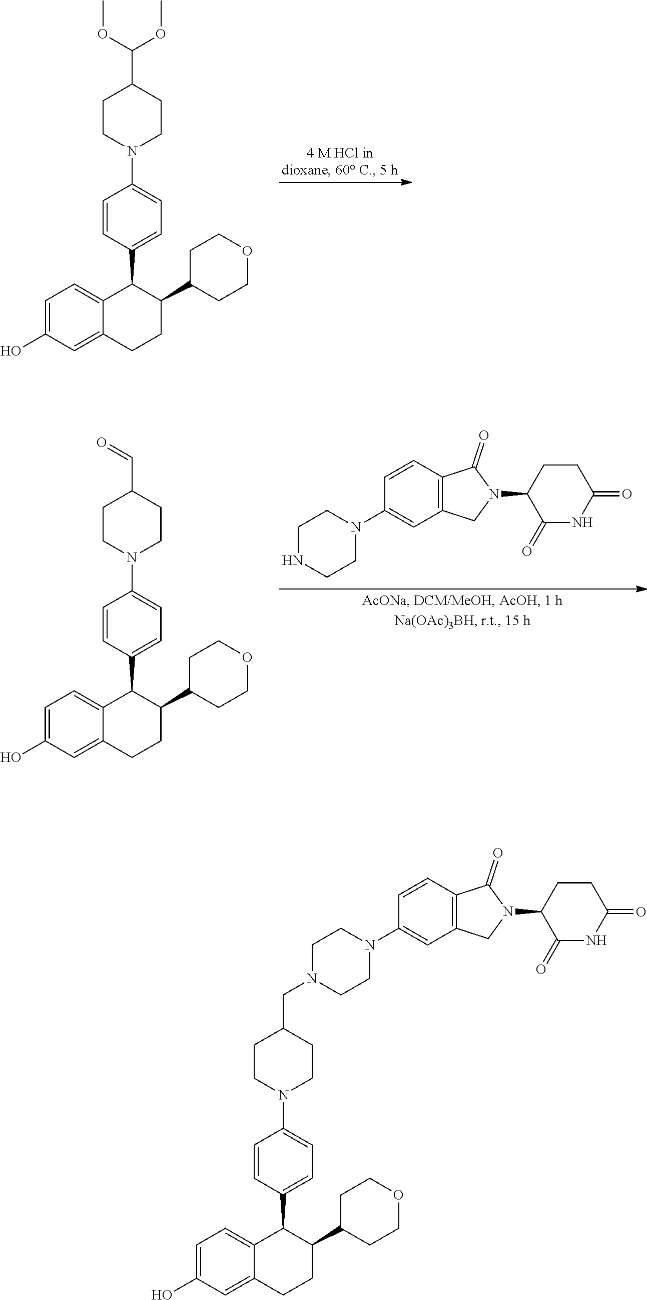 [SMILES: COC(OC)C1CCN(c2ccc([C@@H]3c4ccc(O)cc4CC[C@@H]3C3CCOCC3)cc2)CC1 ; O=CC1CCN(c2ccc([C@@H]3c4ccc(O)cc4CC[C@@H]3C3CCOCC3)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCOCC7)cc6)CC5)CC4)ccc3C2=O)C(=O)N1]

Step 1: To a solution of (1R,2R)-1-[4-[4-(dimethoxymethyl)-1-piperidyl]phenyl]-2-tetrahydropyran-4-yl-tetralin-6-ol (30.00 mg, 64.43 μmol, 1 eq) in HCl/dioxane (1 mL). The mixture was stirred at 60° C. for 5 hr. This reaction mixture was concentrated under reduced pressure to give a residue as a brown solid Compound 1-[4-[(1R,2R)-6-hydroxy-2-tetrahydropyran-4-yl-tetralin-1-yl]phenyl]piperidine-4-carbaldehyde (30 mg, crude) was obtained as a brown solid. This reaction was used to next step without purification.

LC-MS (ESI⁺) m/z: 420.1 (M+H)⁺

¹H NMR (400 MHZ, DMSO-d₆) δ ppm 9.61-9.72 (m, 1H) 7.12-7.32 (m, 2H) 6.97-7.08 (m, 2H) 6.62 (d, J=8.25 Hz, 1H) 6.53 (d, J=2.38 Hz, 1H) 6.43 (dd, J=8.19, 2.44 Hz, 1H) 4.14-4.20 (m, 1H) 3.80-3.91 (m, 2H) 3.69-3.77 (m, 2H) 3.01-3.08 (m, 2H) 2.86-2.96 (m, 2H) 2.69-2.82 (m, 2H) 2.30-2.35 (m, 1H) 2.02-2.14 (m, 3H) 1.71-1.86 (m, 4H) 1.60-1.70 (m, 2H) 1.41-1.55 (m, 2H) 1.20-1.28 (m, 2H)

Step 2: To a solution of 1-[4-[(1R,2R)-6-hydroxy-2-tetrahydropyran-4-yl-tetralin-1-yl]phenyl]piperidine-4-carbaldehyde (25 mg, 59.59 μmol, 1 eq) in mixture of DCM (1 mL) and MeOH (1 mL) was added (3S)-3-(1-oxo-5-piperazin-1-yl-isoindolin-2-yl)piperidine-2,6-dione (29.35 mg, 89.38 μmol, 1.5 eq), HOAc (4.20 mg, 69.94 μmol, 4 μL, 1.17 eq) and NaOAc (15 mg, 182.85 μmol, 3.07 eq) at 25° C. After addition, the mixture was stirred at this temperature for 1 hr, and then NaBH(OAc)₃ (20 mg, 94.37 μmol, 1.58 eq) was added at 25° C. The resulting mixture was stirred at 25° C. for 15 hr. The reaction mixture concentrated under reduced pressure to give a residue as a yellow solid The residue was purified by prep-HPLC (column: Phenomenex luna C18 150*25 mm*10 um; mobile phase: [water(FA)-ACN]; B %: 13%-43%, 58 min). Compound (3S)-3-[5-[4-[[1-[4-[(1R,2R)-6-hydroxy-2-tetrahydropyran-4-yl-tetralin-1-yl]phenyl]-4-piperidyl]methyl]piperazin-1-yl]-1-oxo-isoindolin-2-yl]piperidine-2,6-dione (4 mg, 5.35 μmol, 9.0% yield, 98.0% purity) was obtained as a white solid.

LC-MS (ESI⁺) m/z: 732.3 (M+H)⁺

HPLC: 98.0% purity at 220 nm

SFC: Rt: 1.060 min; Area, 88.684%; method: OJ-3-lPA+ACN(DEA)-40-3ML-35T.

¹H NMR (400 MHZ, DMSO-d₆) δ ppm 10.99 (s, 1H) 8.91-9.08 (m, 1H) δ.15-8.19 (m, 0.573H) 7.49-7.54 (m, 1H) 7.01-7.08 (m, 2H) 6.73-6.85 (m, 4H) 6.58-6.63 (m, 1H) 6.48-6.52 (m, 1H) 6.38-6.44 (m, 1H) 4.98-5.08 (m, 1H) 4.29-4.36 (m, 1H) 4.15-4.23 (m, 1H) 4.05 (br d, J=3.88 Hz, 1H) 3.82-3.88 (m, 1H) 3.70-3.75 (m, 1H) 3.57-3.63 (m, 2H) 3.19 (br d, J=11.63 Hz, 3H) 3.02-3.10 (m, 2H) 2.88 (br dd, J=17.64, 5.13 Hz, 2H) 2.70-2.78 (m, 1H) 2.54-2.61 (m, 3H) 2.31-2.43 (m, 5H) 2.27-2.30 (m, 1H) 2.18-2.23 (m, 2H) 1.92-2.04 (m, 2H) 1.76-1.83 (m, 2H) 1.64-1.74 (m, 2H) 1.54-1.59 (m, 1H) 1.46-1.53 (m, 1H) 1.12-1.25 (m, 5H) 1.03-1.09 (m, 1H)

### Example 259. Preparation of (I-33) (3S)-3-[5-[4-[[1-[4-[(1S,2S)-6-hydroxy-2-tetrahydropyran-4-yl-tetralin-1-yl]phenyl]-4-piperidyl]methyl]piperazin-1-yl]-1-oxo-isoindolin-2-yl]piperidine-2,6-dione

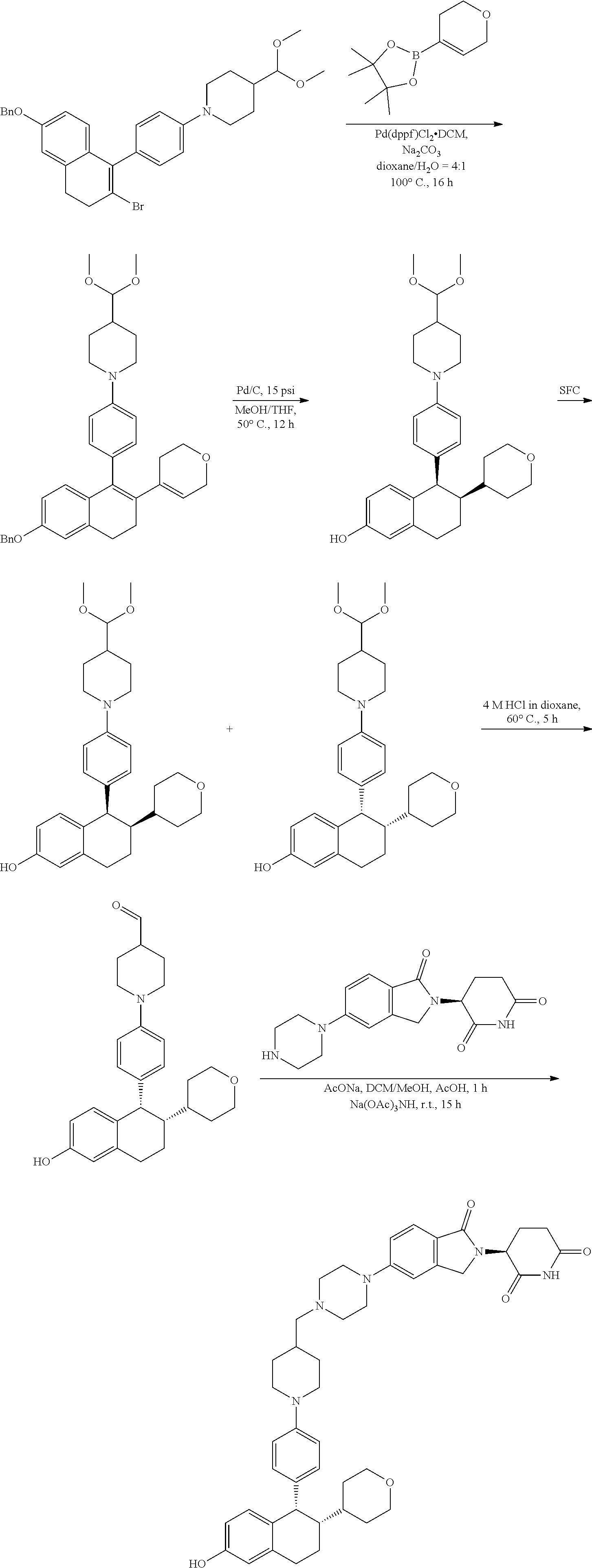

Step 1: A mixture of 1-[4-(6-benzyloxy-2-bromo-3,4-dihydronaphthalen-1-yl)phenyl]-4-(dimethoxymethyl)piperidine (500 mg, 911.5 μmol, 1 eq), 2-(3,6-dihydro-2H-pyran-4-yl)-4,4,5,5-tetramethyl-1,3,2-dioxaborolane (250 mg, 1.19 mmol, 1.3 eq), Pd(dppf)Cl₂ CH₂Cl₂ (372 mg, 455.78 μmol, 0.5 eq), Na₂CO₃ (290 mg, 2.73 mmol, 3 eq) in dioxane (4 mL) and Water (1 mL) was degassed and purged with N₂ for 3 times, and then the mixture was stirred at 100° C. for 16 hr under N₂ atmosphere. The reaction mixture was concentrated under reduced pressure to remove dioxane and water. The residue was purified by flash silica gel chromatography (ISCO®; 12 g SepaFlash® Silica Flash Column, Eluent of 0˜30% Ethyl acetate/Petroleum ether @ 40 mL/min). Compound 1-[4-[6-benzyloxy-2-(3,6-dihydro-2H-pyran-4-yl)-3,4-dihydronaphthalen-1-yl]phenyl]-4-(dimethoxymethyl)piperidine (300 mg, 518.23 μmol, 56.8% yield, 95.3% purity) was obtained as a yellow solid.

LC-MS (ESI+) m/z: 552.4 (M+H)⁺

Step 2: A mixture of 1-[4-[6-benzyloxy-2-(3,6-dihydro-2H-pyran-4-yl)-3,4-dihydronaphthalen-1-yl]phenyl]-4-(dimethoxymethyl)piperidine (300.00 mg, 543.76 μmol, 1 eq), Pd/C (200 mg, 2.72 mmol, 10% purity, 5 eq) in MeOH (2 mL) and THF (2 mL) was degassed and purged with H₂ for 3 times, and then the mixture was stirred at 50° C. for 12 hr under H₂ (15 psi) atmosphere. The reaction mixture was filtered and the filter cake was rinsed with 50 mL of MeOH, The filter was collected, dried in vacuo to give a colorless oil. The residue was purified by flash silica gel chromatography (ISCOR; 12 g SepaFlash® Silica Flash Column, Eluent of 0˜50% Ethyl acetate/Petroleum ether @ 40 mL/min) to give desired a colorless oil.

Which was further separated by SFC (condition: column: Welch Ultimate C18 150*25 mm*5 um; mobile phase: [water(FA)-ACN]; B %: 20%-50%, 10 min) and condition (column: Welch Ultimate C18 150*25 mm*5 um; mobile phase: [water(FA)-ACN]; B %: 18%-48%, 10 min). Compound (1S,2S)-1-[4-[4-(dimethoxymethyl)-1-piperidyl]phenyl]-2-tetrahydropyran-4-yl-tetralin-6-ol (95 mg, 197.50 μmol, 36.32% yield, 96.8% purity) was obtained as a colorless oil. Compound (1R,2R)-1-[4-[4-(dimethoxymethyl)-1-piperidyl]phenyl]-2-tetrahydropyran-4-yl-tetralin-6-ol (96 mg, 197.93 μmol, 36.40% yield, 96% purity) was obtained as a colorless oil.

LC-MS (ESI⁺) m/z: 466.2 (M+H)⁺

SFC for (1S,2S)-1-[4-[4-(dimethoxymethyl)-1-piperidyl]phenyl]-2-tetrahydropyran-4-yl-tetralin-6-ol: Rt: 2.055 min; Area, 99.885%;

SFC for (1R,2R)-1-[4-[4-(dimethoxymethyl)-1-piperidyl]phenyl]-2-tetrahydropyran-4-yl-tetralin-6-ol: Rt: 2.165 min; Area, 98.111%;

Step 3: To a solution of (1S,2S)-1-[4-[4-(dimethoxymethyl)-1-piperidyl]phenyl]-2-tetrahydropyran-4-yl-tetralin-6-ol (30 mg, 64.43 μmol, 1 eq) in HCl/dioxane (1 mL). The mixture was stirred at 60° C. for 5 hr. This reaction mixture was concentrated under reduced pressure to give a residue as a brown solid. Compound 1-[4-[(1S,2S)-6-hydroxy-2-tetrahydropyran-4-yl-tetralin-1-yl]phenyl]piperidine-4-carbaldehyde (30 mg, crude) was obtained as a brown solid. This reaction was used to next step without purification.

LC-MS (ESI⁺) m/z: 420.0 (M+H)⁺¹H NMR (400 MHZ, DMSO-d₆) δ ppm 9.57-9.72 (m, 1H) 7.33-7.58 (m, 2H) 7.07-7.18 (m, 2H) 6.62 (d, J=8.25 Hz, 1H) 6.54 (d, J=2.38 Hz, 1H) 6.43 (dd, J=8.32, 2.44 Hz, 1H) 3.80-3.88 (m, 2H) 3.69-3.76 (m, 2H) 3.13-3.23 (m, 4H) 2.98-3.08 (m, 2H) 2.63-2.78 (m, 3H) 2.07-2.19 (m, 2H) 1.84-1.99 (m, 3H) 1.71-1.72 (m, 1H) 1.61-1.71 (m, 2H) 1.39-1.54 (m, 2H) 1.15-1.17 (m, 1H)

Step 4: To a solution of 1-[4-[(1S,2S)-6-hydroxy-2-tetrahydropyran-4-yl-tetralin-1-yl]phenyl]piperidine-4-carbaldehyde (30.00 mg, 71.50 μmol, 1 eq) in mixture of DCM (0.5 mL) and MeOH (0.5 mL) was added (3S)-3-(1-oxo-5-piperazin-1-yl-isoindolin-2-yl)piperidine-2,6-dione (50.11 mg, 100.11 μmol, 1.40 eq, p-TSA salt), HOAc (4.20 mg, 69.94 μmol, 4.00 uL, 1 eq) and NaOAc (18.00 mg, 219.43 μmol, 3.07 eq) at 25° C. After addition, the mixture was stirred at this temperature for 1 hr, and then NaBH(OAc)₃ (23.00 mg, 108.52 μmol, 1.52 eq) was added at 25° C. The resulting mixture was stirred at 25° C. for 15 hr. The reaction mixture concentrated under reduced pressure to give a residue as a yellow solid. The residue was purified by prep-HPLC (column: Phenomenex luna C18 150*25 mm*10 um; mobile phase: [water(FA)-ACN]; B %: 13%-43%, 58 min). Compound (3S)-3-[5-[4-[ ]-[4-[(1S,2S)-6-hydroxy-2-tetrahydropyran-4-yl-tetralin-1-yl]phenyl]-4-piperidyl]methyl]piperazin-1-yl]-1-oxo-isoindolin-2-yl]piperidine-2,6-dione (8 mg, 10.77 μmol, 15.07% yield, 98.560% purity) was obtained as a white solid.

LC-MS (ESI+) m/z: 732.3 (M+H)⁺

HPLC: 98.560% purity at 220 nm

SFC: Rₜ: 0.864 min; Area, 92.143%; ¹H NMR (400 MHZ, DMSO-d₆) δ ppm 10.89-10.98 (m, 1H) δ.98-9.05 (m, 1H) δ.13-8.15 (m, 0.332H) 7.49-7.54 (m, 1H) 7.04-7.09 (m, 2H) 6.74-6.85 (m, 4H) 6.62 (d, J=8.38 Hz, 1H) 6.49-6.51 (m, 1H) 6.38-6.43 (m, 1H) 5.01-5.08 (m, 1H) 4.29-4.36 (m, 1H) 4.16-4.23 (m, 1H) 4.04 (br d, J=4.13 Hz, 1H) 3.82-3.88 (m, 1H) 3.70-3.76 (m, 1H) 3.55-3.64 (m, 2H) 3.29 (br d, J=3.75 Hz, 5H) 3.14-3.21 (m, 1H) 3.01-3.10 (m, 1H) 2.82-2.92 (m, 2H) 2.64-2.79 (m, 2H) 2.56-2.62 (m, 3H) 2.28-2.43 (m, 2H) 2.18-2.24 (m, 2H) 1.92-2.04 (m, 2H) 1.76-1.82 (m, 2H) 1.63-1.75 (m, 2H) 1.31-1.62 (m, 3H) 1.13-1.27 (m, 5H) 1.02-1.11 (m, 1H)

### Example 260. Preparation of (I-29) (S)-3-(5-(4-((1-(4-((1S,2R)-6-hydroxy-2-(1-methyl-1H-pyrazol-3-yl)-1,2,3,4-tetrahydronaphthalen-1-yl)phenyl)piperidin-4-yl)methyl)piperazin-1-yl)-1-oxoisoindolin-2-yl)piperidine-2,6-dione

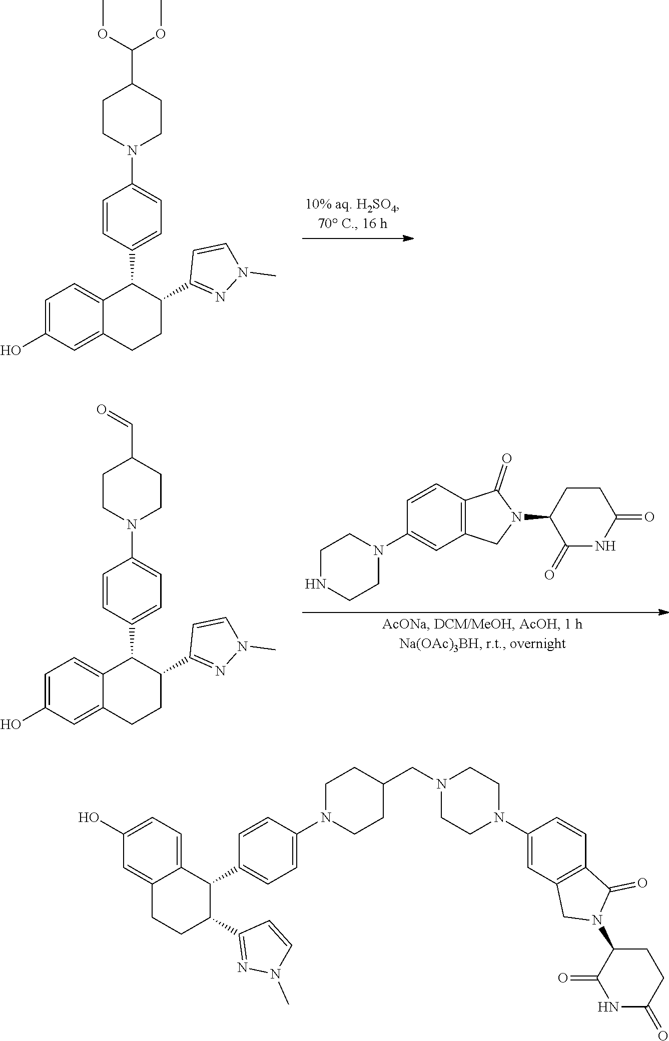 [SMILES: COC(OC)C1CCN(c2ccc([C@H]3c4ccc(O)cc4CC[C@H]3c3ccn(C)n3)cc2)CC1 ; Cn1ccc([C@@H]2CCc3cc(O)ccc3[C@@H]2c2ccc(N3CCC(C=O)CC3)cc2)n1 ; O=C1CC[C@H](N2Cc3cc(N4CCNCC4)ccc3C2=O)C(=O)N1 ; Cn1ccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n1]

Step 1: To a solution of (5S,6R)-5-(4-(4-(dimethoxymethyl)piperidin-1-yl)phenyl)-6-(1-methyl-1H-pyrazol-3-yl)-5,6,7,8-tetrahydronaphthalen-2-ol (100 mg, 1 Eq, 217 μmol) in THF (15 mL) was added sulfuric acid (212 mg, 15 mL, 10% Wt., 1 Eq, 217 μmol). The mixture was stirred at 70° C. for 4 hour. LCMS showed 100% desired MS. The mixture was adjusted to pH 8-9 by progressively adding NaHCO₃. The reaction mixture was quenched by addition 20 mL EtOAc at 20° C., and then diluted with 20 mL H₂O and extracted with 50 mL (25 mL*2) EtOAc. The combined organic layers were washed with 10 mL (10 mL*1) and dried over Na₂SO₄. The reaction was filtered and concentrated under reduced pressure to give 1-(4-((1S, 2R)-6-hydroxy-2-(1-methyl-1H-pyrazol-3-yl)-1, 2, 3, 4-tetrahydronaphthalen-1-yl)phenyl)piperidine-4-carbaldehyde (100 mg, 241 μmol, 111%, 100% Purity) was obtained as a brown solid. LC-MS (ESI⁺) m/z: 434.0 (M+H)⁺.

Step 2: A mixture of 1-(4-((1S,2R)-6-hydroxy-2-(1-methyl-1H-pyrazol-3-yl)-1,2,3,4-tetrahydronaphthalen-1-yl)phenyl)piperidine-4-carbaldehyde (100 mg, 1 Eq, 241 μmol), (R)-3-(1-oxo-5-(piperazin-1-yl)isoindolin-2-yl)piperidine-2,6-dione, TsOH salt (142 mg, 84.8% Wt., 1 Eq. 241 μmol) and sodium acetate (98.7 mg, 5 Eq, 1.20 mmol) in DCM (1.5 mL) and MeOH (1.5 mL) at 20° C. for 0.5 h, pH=8, then Acetic acid (43.4 mg, 41.5 uL, 3 Eq, 722 μmol) at 20° C. for 1 h, pH-6, then added NaBH(OAc)₃ (30.2 mg, 2 Eq, 481 μmol) in the mixture, and then the mixture was stirred at 25° C. for 16 h. LCMS showed 72.41% desired MS. The reaction was concentrated to give (S)-3-(5-(4-((1-(4-((1S,2R)-6-hydroxy-2-(1-methyl-1H-pyrazol-3-yl)-1,2,3,4-tetrahydronaphthalen-1-yl)phenyl)piperidin-4-yl)methyl)piperazin-1-yl)-1-oxoisoindolin-2-yl)piperidine-2,6-dione (49.3 g, 62.6 mmol, 26000%, 92.4% Purity) as a brown solid.

LCMS: calc. for C₄₃H₄₉N₇O₄:727.89, found: [M+H]⁺ 728.2.

HPLC: 92.4% purity at 220 nm.

¹H NMR (400 MHZ, DMSO-d₆) δ=11.00-10.89 (m, 1H), 9.12-9.02 (m, 1H), 8.17-8.12 (m, 0.525H), 7.55-7.50 (m, 1H), 7.49-7.46 (m, 1H), 7.42-7.36 (m, 1H), 7.13-7.05 (m, 2H), 6.68-6.56 (m, 4H), 6.48-6.36 (m, 3H), 5.52-5.46 (m, 1H), 5.10-5.00 (m, 1H), 4.38-4.29 (m, 1H), 4.25-4.18 (m, 2H), 3.77-3.72 (m, 3H), 3.60-3.48 (m, 3H), 3.28-3.17 (m, 4H), 2.95-2.85 (m, 3H), 2.68-2.53 (m, 6H), 2.43-2.27 (m, 4H), 2.01-1.90 (m, 2H), 1.84-1.65 (m, 4H), 1.23-1.12 (m, 2H)

SFC: retention time, 2.599 min; Area, 94.885%; Stereochemistry at the ERBM core is arbitrarily assigned.

### Example 261. Preparation of (I-28) (S)-3-(5-(4-((1-(4-((1R,2S)-6-hydroxy-2-(1-methyl-1H-pyrazol-3-yl)-1,2,3,4-tetrahydronaphthalen-1-yl)phenyl)piperidin-4-yl)methyl)piperazin-1-yl)-1-oxoisoindolin-2-yl)piperidine-2,6-dione

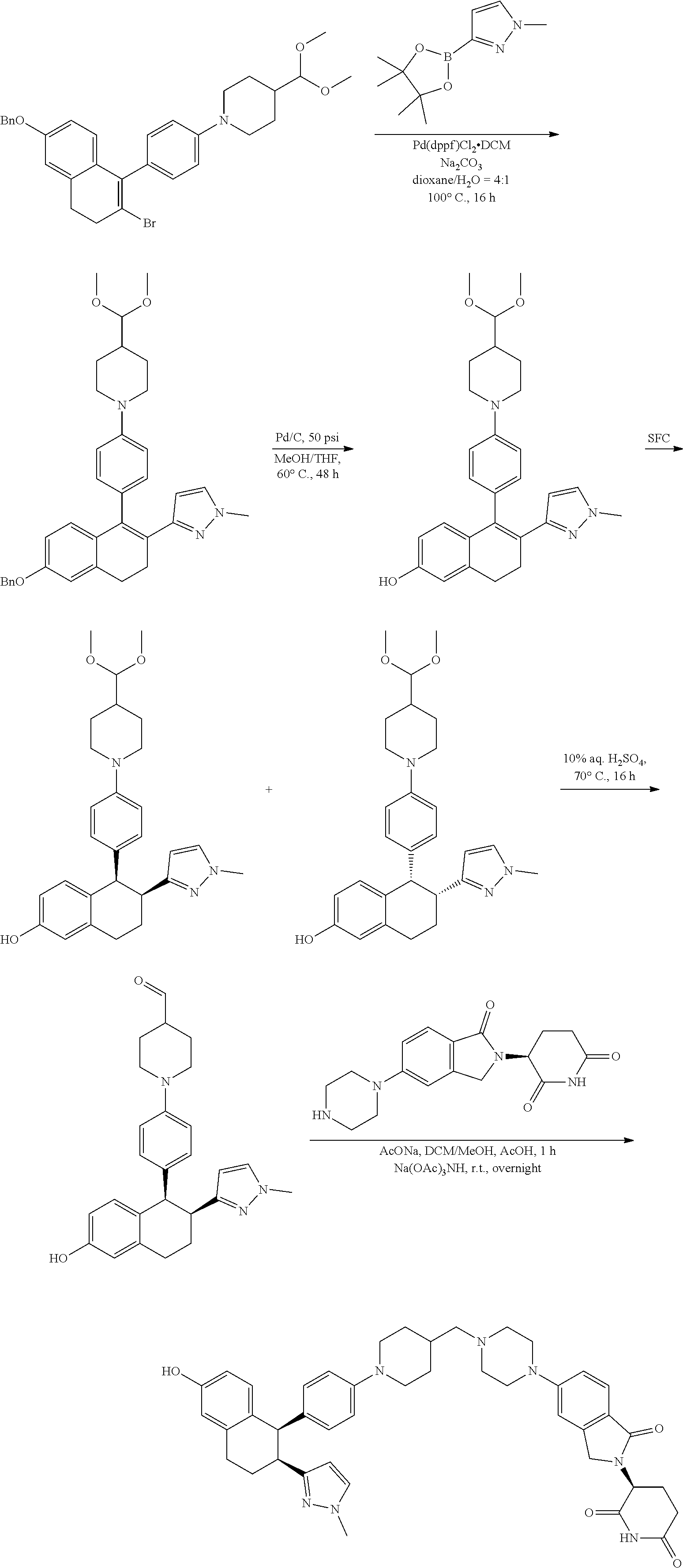

Step 1: A mixture of 1-[4-(6-benzyloxy-2-bromo-3,4-dihydronaphthalen-1-yl)phenyl]-4-(dimethoxymethyl)piperidine (500 mg, 911.56 μmol, 1 eq), 1-methyl-3-(4,4,5,5-tetramethyl-1,3,2-dioxaborolan-2-yl)pyrazole (246 mg, 1.19 mmol, 1.3 eq), Pd(dppf)Cl₂·CH₂Cl₂ (372 mg, 455.78 mol, 0.5 eq), Na₂CO₃ (290 mg, 2.73 mmol, 3 eq) in dioxane (4 mL) and Water (1 mL) was degassed and purged with N₂ for 3 times, and then the mixture was stirred at 100° C. for 16 h under N₂ atmosphere. The reaction mixture was concentrated under reduced pressure to remove dioxane and H2O, The residue was purified by flash silica gel chromatography (ISCOR 12 g SepaFlash® Silica Flash Column, Eluent of 0˜30% Ethyl acetate/Petroleum ether @ 40 mL/min). Compound 1-[4-[6-benzyloxy-2-(1-methylpyrazol-3-yl)-3, 4-dihydronaphthalen-1-yl]phenyl]-4-(dimethoxymethyl)piperidine (300 mg, 438.64 μmol, 48.12% yield, 80.37% purity) was obtained as a yellow solid. LC-MS (ESI+) m/z: 550.5 (M+H)⁺

Step 2: A mixture of 1-[4-[6-benzyloxy-2-(1-methylpyrazol-3-yl)-3,4-dihydronaphthalen-1-yl]phenyl]-4-(dimethoxymethyl)piperidine (800 mg, 1.46 mmol, 1 eq), Pd/C (1 g, 7.28 mmol, 10% purity, 5 eq) in MeOH (2 mL) and THF (2 mL) was degassed and purged with H₂ for 3 times, and then the mixture was stirred at 60° C. for 48 h under H₂ (50 psi) atmosphere, The reaction mixture was filtered and the filter cake was rinsed with 50 mL of DCM, The filter was concentrated to dryness in vacuo. The residue was purified by flash silica gel chromatography (ISCO®; 12 g SepaFlash® Silica Flash Column, Eluent of 0˜70% Ethyl acetate/Petroleum ether @ 50 mL/min) to give desired compound (400 mg, purity 96.808%) as a colorless oil, which was further separated by SFC (column: DAICEL CHIRALCEL OJ (250 mm*30 mm, 10 um); mobile phase: [0.1% NH₃H₂O MEOH]; B %: 25%-25%, C7.5; 120 min).

LC-MS (ESI+) m/z: 462.3 (M+H)⁺

Compound (1R, 2S)-1-[4-[4-(dimethoxymethyl)-1-piperidyl]phenyl]-2-(1-methylpyrazol-3-yl)tetralin-6-ol (190 mg, 370.46 μmol, 25.45% yield, 90% purity) was obtained as a colorless oil. Compound (1S, 2R)-1-[4-[4-(dimethoxymethyl)-1-piperidyl]phenyl]-2-(1-methylpyrazol-3-yl)tetralin-6-ol (190 mg, 370.46 μmol, 25.45% yield, 90% purity) was obtained as a colorless oil.

Step 3: To a solution of (5R,6S)-5-(4-(4-(dimethoxymethyl)piperidin-1-yl)phenyl)-6-(1-methyl-1H-pyrazol-3-yl)-5,6,7,8-tetrahydronaphthalen-2-ol (100 mg, 1 Eq, 217 μmol) in THF (15 mL) was added sulfuric acid (212 mg, 15 mL, 10% Wt., 1 Eq, 217 μmol). The mixture was stirred at 70° C. for 16 hour. LCMS showed 97.9% desired MS. The mixture was adjusted to pH 8-9 by progressively adding NaHCO₃. The reaction mixture was quenched by addition 20 mL EtOAc at 20° C., and then diluted with 20 mL H₂O and extracted with 50 mL (25 mL*2) EtOAc. The combined organic layers were washed with 10 mL (10 mL*1) and dried over Na₂SO₄. The reaction was filtered and concentrated under reduced pressure to give 1-(4-((1R, 2S)-6-hydroxy-2-(1-methyl-1H-pyrazol-3-yl)-1, 2, 3, 4-tetrahydronaphthalen-1-yl)phenyl)piperidine-4-carbaldehyde (103 mg, 243 μmol, 112%, 97.9% Purity) was obtained as a brown solid. LC-MS (ESI⁺) m/z: 434.0 (M+H)⁺.

Step 4: A mixture of 1-(4-((1R,2S)-6-hydroxy-2-(1-methyl-1H-pyrazol-3-yl)-1,2,3,4-tetrahydronaphthalen-1-yl)phenyl)piperidine-4-carbaldehyde, (R)-3-(1-oxo-5-(piperazin-1-yl)isoindolin-2-yl)piperidine-2,6-dione, TsOH acid (142 mg, 84.8% Wt., 1 Eq, 241 μmol) and Sodium acetate (98.7 mg, 5 Eq, 1.20 mmol) in DCM (1.5 mL) and MeOH (1.5 mL) at 20° C. for 0.5 h, then Acetic acid (43.4 mg, 41.5 uL, 3 Eq, 722 μmol) at 20° C. for 1 h, then added NaBH(OAc) 3 (30.2 mg, 2 Eq, 481 μmol) the mixture, and then the mixture was stirred at 25° C. for 16 h. LCMS showed 96.2% desired MS. The reaction was concentrated to give (S)-3-(5-(4-((1-(4-((1R,2S)-6-hydroxy-2-(1-methyl-1H-pyrazol-3-yl)-1,2,3,4-tetrahydronaphthalen-1-yl)phenyl)piperidin-4-yl)methyl)piperazin-1-yl)-1-oxoisoindolin-2-yl)piperidine-2,6-dione (59.6 mg, 78.2 μmol, 32.5%, 95.5% Purity) as a brown solid.

LCMS: calc. for C₄₃H₄₉N₇O₄:727.89, found: [M+H]⁺ 728.2.

HPLC: 95.517% purity at 220 nm.

¹H NMR (400 MHZ, DMSO-d₆) δ=11.01-10.87 (m, 1H), 9.17-9.04 (m, 1H), 8.16-8.13 (m, 0.446H), 7.55-7.47 (m, 1H), 7.39 (d, J=2.0 Hz, 1H), 7.14-7.05 (m, 1H), 6.69-6.55 (m, 3H), 6.50-6.38 (m, 3H), 5.53-5.45 (m, 1H), 5.11-5.00 (m, 1H), 4.37-4.30 (m, 1H), 4.26-4.17 (m, 2H), 3.76-3.72 (m, 3H), 3.53 (br d, J=5.4 Hz, 3H), 3.29-3.18 (m, 4H), 2.96-2.85 (m, 3H), 2.70-2.52 (m, 8H), 2.47-2.24 (m, 4H), 2.01-1.89 (m, 2H), 1.86-1.62 (m, 3H), 1.18 (brd, J=9.8 Hz, 2H)

SFC: retention time, 3.129 min; Area, 95.074%; Stereochemistry at the ERBM core is arbitrarily assigned.

### Example 262. Preparation of (I-25) (S)-3-(5-(4-((1-(4-((1S,2R)-6-hydroxy-2-(1-methyl-1H-pyrazol-5-yl)-1,2,3,4-tetrahydronaphthalen-1-yl)phenyl)piperidin-4-yl)methyl)piperazin-1-yl)-1-oxoisoindolin-2-yl)piperidine-2,6-dione

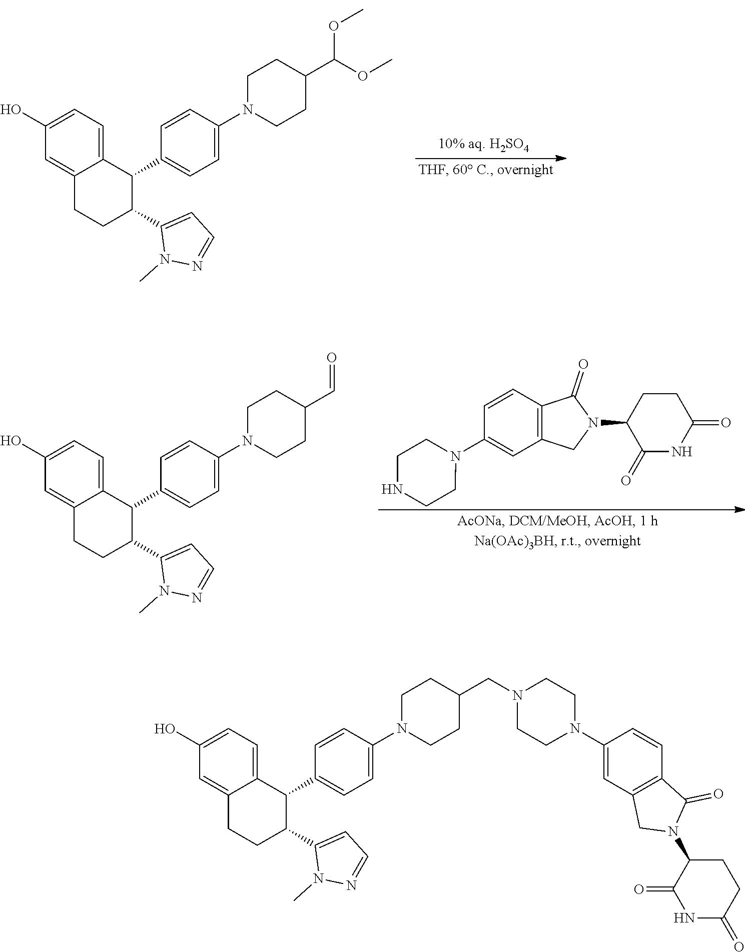

Step 1: A mixture of (5S,6R)-5-(4-(4-(dimethoxymethyl)piperidin-1-yl)phenyl)-6-(1-methyl-1H-pyrazol-5-yl)-5,6,7,8-tetrahydronaphthalen-2-ol (100 mg, 97% Wt, 1 Eq, 217 μmol) in 10% H₂SO₄ (1.5 mL) and THF (1.5 mL), then the mixture was stirred at 60° C. for 16 hour. The solvent was removed under vacuum. The residue was dissolved in water (20 mL) and washed by ethyl acetate (20 mL). The aqueous solution was added NaHCO₃ to adjust pH=6. The combined organic layer was dried over Na₂SO₄, filtered and concentrated under vacuum to give 1-(4-((1S,2R)-6-hydroxy-2-(1-methyl-1H-pyrazol-5-yl)-1,2,3,4-tetrahydronaphthalen-1-yl)phenyl)piperidine-4-carbaldehyde (70 mg, 0.17 mmol, 80%) as a yellow oil. LC-MS (ESI+) m/z: 416.3 (M+H)+

Step 2: A mixture of 1-(4-(1S,2R)-6-hydroxy-2-(1-methyl-1H-pyrazol-5-yl)-1,2,3,4-tetrahydronaphthalen-1-yl)phenyl)piperidine-4-carbaldehyde (70 mg, 1 Eq, 0.17 mmol), (R)-3-(1-oxo-5-(piperazin-1-yl)isoindolin-2-yl)piperidine-2,6-dione, Tosylic acid (110 mg, 1.3 Eq, 0.22 mmol) and Sodium acetate (69 mg, 5 Eq, 0.84 mmol) and Acetic acid (30 mg, 29 μL, 3 Eq, 0.50 mmol) in DCM (2 mL) and MeOH (2 mL) at 25° C. for 1 hour, then Sodium triacetoxyborohydride (71 mg, 2 Eq, 0.34 mmol) was added to the mixture and was stirred at 25° C. for 16 hour. The reaction was treated with 12O (20 mL), extracted with EtOAc (20 mL). The combined extracts was dried over anhydrous Na₂SO₄, filtered and concentrated to dryness in vacuo to give a residue. The residue was purified by prep.HPLC (column: Welch Xtimate C18 150*30 mm*5 um; mobile phase: [water(FA)-ACN]; B %: 10%-40%, 7 min). The aqueous phase was lyophilized to dryness to give (S)-3-(5-(4-((1-(4-((1S,2R)-6-hydroxy-2-(1-methyl-1H-pyrazol-5-yl)-1,2,3,4-tetrahydronaphthalen-1-yl)phenyl)piperidin-4-yl)methyl)piperazin-1-yl)-1-oxoisoindolin-2-yl)piperidine-2,6-dione (58.9 mg, 62.4 μmol, 52% yield, 97.521% purity) as a white solid.

LC-MS (ESI+) m/z: 728.4 (M+H)+

HPLC: 97.521%, purity at 220 nm.

SFC: retention time, 3.311 min; Area, 87.483%; Stereochemistry at the ERBM core is arbitrarily assigned.

¹H NMR (400 MHZ, DMSO-d₆) δ=11.01-10.89 (m, 1H), 9.25-9.02 (m, 1H), 8.20-8.14 (m, 0.279H), 7.58-7.47 (m, 1H), 7.13-7.01 (m, 3H), 6.69-6.66 (m, 1H), 6.63-6.55 (m, 3H), 6.52-6.48 (m, 1H), 6.33-6.22 (m, 2H), 5.29-5.24 (m, 1H), 5.09-5.01 (m, 1H), 4.37-4.29 (m, 1H), 4.28-4.17 (m, 2H), 3.91-3.84 (m, 3H), 3.58-3.49 (m, 2H), 3.29 (br d, J=3.7 Hz, 13H), 2.98-2.84 (m, 3H), 2.27-2.16 (m, 2H), 2.05-1.92 (m, 2H), 1.81-1.59 (m, 4H), 1.27-1.07 (m, 2H)

### Example 263. Preparation of (I-24) (S)-3-(5-(4-((1-(4-((1R,2S)-6-hydroxy-2-(1-methyl-1H-pyrazol-5-yl)-1,2,3,4-tetrahydronaphthalen-1-yl)phenyl)piperidin-4-yl)methyl)piperazin-1-yl)-1-oxoisoindolin-2-yl)piperidine-2,6-dione

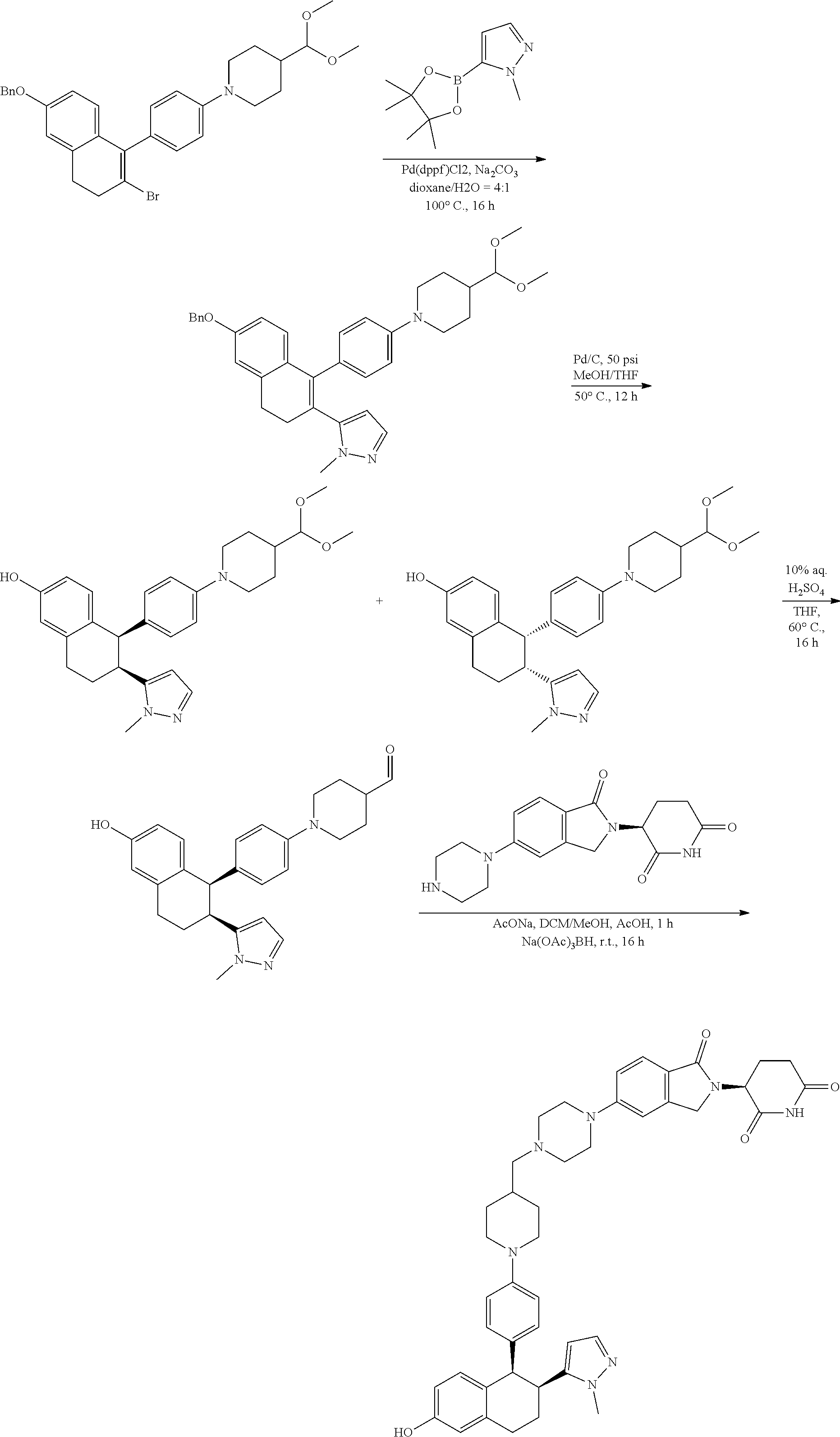

Step 1: A mixture of 1-(4-(6-(benzyloxy)-2-bromo-3,4-dihydronaphthalen-1-yl)phenyl)-4-(dimethoxymethyl)piperidine (1.5 g, 1 Eq, 2.7 mmol), 1-methyl-5-(4,4,5,5-tetramethyl-1,3,2-dioxaborolan-2-yl)-1H-pyrazole (0.63 g, 1.1 Eq, 3.0 mmol), Na₂CO₃ (0.87 g, 3 Eq, 8.2 mmol) and [1,1′-Bis(diphenylphosphino)ferrocene]dichloropalladium(II) complex with dichloromethane (0.22 g, 0.1 Eq, 0.27 mmol) in Dioxane (16 mL) and H₂O (4.0 mL) at r.t. N₂ was bubbled into the mixture for 5 min. The reaction mixture was heated at 100° C. for 16 hour. Then the reaction was cooled to room temperature. The mixture was treated with H₂O (50 mL), extracted with EtOAc (50 mL). The combined extracts was dried over anhydrous Na₂SO₄, filtered and concentrated to dryness in vacuo to give a residue. The residue was subjected to column chromatography over silica gel (gradient elution: 0˜20% ethyl acetate in petroleum ether). The pure fractions were collected and concentrated to dryness in vacuo to give 1-(4-(6-(benzyloxy)-2-(1-methyl-1H-pyrazol-5-yl)-3,4-dihydronaphthalen-1-yl)phenyl)-4-(dimethoxymethyl)piperidine (600 mg, 1.0 mmol, 38%, 96% Purity) as a white solid. LC-MS (ESI⁺) m/z: 550.3 (M+H)⁺.

Step 2: A mixture of 1-(4-(6-(benzyloxy)-2-(1-methyl-1H-pyrazol-5-yl)-3,4-dihydronaphthalen-1-yl)phenyl)-4-(dimethoxymethyl)piperidine (600 mg, 96% Wt, 1 Eq, 1.05 mmol), H2 (2.12 mg, 1 Eq, 1.05 mmol), Pd/C (600 mg, 10% Wt, 0.538 Eq, 564 μmol) in MeOH (1.5 mL), THF (1.5 mL) was degassed and purged with H₂ for 3 times and then the mixture was stirred at 50 psi, 50° C. for 12 hour under H₂ atmosphere. The reaction was filtered and concentrated to dryness in vacuo to give a white solid. The white solid was subjected by SFC: Column: DAICEL CHIRALCEL OD (250 mm*30 mm, 10 um); Mobile phase: A: Supercritical CO₂, B: MeOH (0.1% NH; H2O), A:B=60:40 at 80 mL/min; The aqueous phase was lyophilized to dryness to give (5R,6S)-5-(4-(4-(dimethoxymethyl)piperidin-1-yl)phenyl)-6-(1-methyl-1H-pyrazol-5-yl)-5,6,7,8-tetrahydronaphthalen-2-ol (130 mg, 282 μmol, 26.9%, 100% Purity) and (5S,6R)-5-(4-(4-(dimethoxymethyl)piperidin-1-yl)phenyl)-6-(1-methyl-1H-pyrazol-5-yl)-5,6,7,8-tetrahydronaphthalen-2-ol (130 mg, 0.27 mmol, 26%, 97% Purity) as a yellow oil. LC-MS (ESI⁺) m/z: 462.2 (M+H)⁺.

Step 3: A mixture of (5R,6S)-5-(4-(4-(dimethoxymethyl)piperidin-1-yl)phenyl)-6-(1-methyl-1H-pyrazol-5-yl)-5,6,7,8-tetrahydronaphthalen-2-ol (100 mg, 100% Wt, 1 Eq, 217 μmol) in 10% H₂SO₄ (1.5 mL) and THF (1.5 mL), then the mixture was stirred at 60° C. for 16 hour. The solvent was removed under vacuum. The residue was dissolved in water (20 mL) and washed by ethyl acetate (20 mL). The aqueous solution was added NaHCO₃ to adjust pH=6. The combined organic layer was dried over Na₂SO₄, filtered and concentrated under vacuum to give 1-(4-((1R,2S)-6-hydroxy-2-(1-methyl-1H-pyrazol-5-yl)-1,2,3,4-tetrahydronaphthalen-1-yl)phenyl)piperidine-4-carbaldehyde (70 mg, 0.17 mmol, 78%) as a yellow oil. LC-MS (ESI+) m/z: 416.2 (M+H)+

Step 4: A mixture of 1-(4-((1R,2S)-6-hydroxy-2-(1-methyl-1H-pyrazol-5-yl)-1,2,3,4-tetrahydronaphthalen-1-yl)phenyl)piperidine-4-carbaldehyde (70 mg, 1 Eq, 0.17 mmol), (R)-3-(1-oxo-5-(piperazin-1-yl)isoindolin-2-yl)piperidine-2,6-dione, Tosylic acid (110 mg, 1.3 Eq, 0.22 mmol) and Sodium acetate (69 mg, 5 Eq, 0.84 mmol) and Acetic acid (30 mg, 29 μL, 3 Eq. 0.5 mmol) in DCM (2 mL) and MeOH (2 mL) at 25° C. for 1 hour, then Sodium triacetoxyborohydride (71 mg, 2 Eq, 0.34 mmol) was added to the mixture. Then the reaction was stirred at 25° C. for 16 hour. The reaction was treated with H₂O (20 mL), extracted with EtOAc (20 mL). The combined extracts was dried over anhydrous Na₂SO₄, filtered and concentrated to dryness in vacuo to give a residue. The residue was purified by prep HPLC (column: Welch Xtimate C18 150*30 mm*5 um; mobile phase: [water(FA)-ACN]; B %: 9%-39%, 7 min). The aqueous phase was lyophilized to dryness to give (S)-3-(5-(4-((1-(4-((1R,2S)-6-hydroxy-2-(1-methyl-1H-pyrazol-5-yl)-1,2,3,4-tetrahydronaphthalen-1-yl)phenyl)piperidin-4-yl)methyl)piperazin-1-yl)-1-oxoisoindolin-2-yl)piperidine-2,6-dione (46.6 mg, 62.4 μmol, 41% yield, 97.536% purity) as a white solid.

LC-MS (ESI+) m/z: 728.4 (M+H)+

HPLC: 97.536%, purity at 220 nm.

SFC: retention time, 3.287 min; Arca, 91.314%; Stereochemistry at the ERBM core is arbitrarily assigned.

¹H NMR (400 MHZ, DMSO-d₆) δ=10.99-10.92 (m, 1H), 9.41-8.86 (m, 1H), 8.29-8.02 (m, 0.290H), 7.58-7.48 (m, 1H), 7.13-6.99 (m, 3H), 6.70-6.64 (m, 1H), 6.62-6.55 (m, 3H), 6.53-6.47 (m, 1H), 6.32-6.24 (m, 2H), 5.28-5.24 (m, 1H), 5.11-4.99 (m, 1H), 4.39-4.28 (m, 1H), 4.28-4.17 (m, 2H), 3.90-3.84 (m, 3H), 3.59-3.48 (m, 3H), 3.43-3.22 (m, 12H), 2.97-2.84 (m, 3H), 2.25-2.15 (m, 2H), 2.07-1.91 (m, 2H), 1.82-1.58 (m, 4H), 1.25-1.10 (m, 2H).

### Example 264. Preparation of (I-413) (3.5)-3-[5-[4-[[1-[4-[(1S,2R)-6-hydroxy-2-indan-5-yl-tetralin-1-yl]phenyl]-4-piperidyl]methyl]piperazin-1-yl]-1-oxo-isoindolin-2-yl]piperidine-2,6-dione

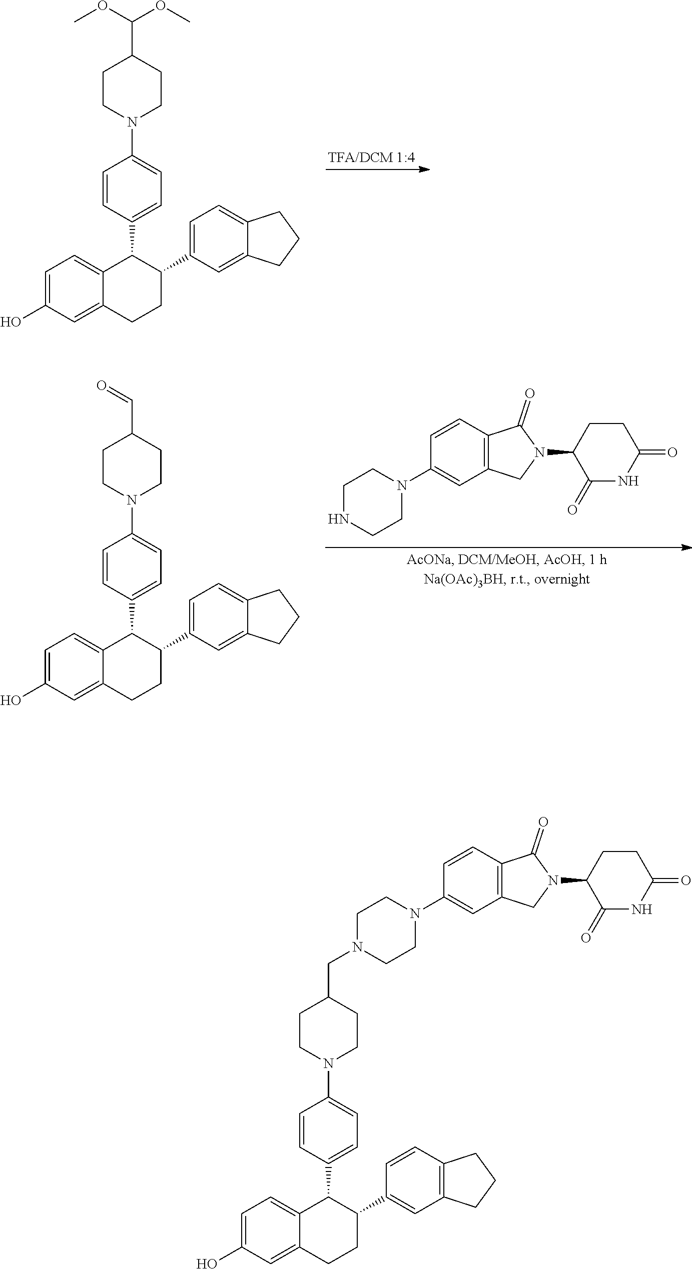 [SMILES: COC(OC)C1CCN(c2ccc([C@H]3c4ccc(O)cc4CC[C@H]3c3ccc4c(c3)CCC4)cc2)CC1 ; O=CC1CCN(c2ccc([C@H]3c4ccc(O)cc4CC[C@H]3c3ccc4c(c3)CCC4)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc8c(c7)CCC8)cc6)CC5)CC4)ccc3C2=O)C(=O)N1]

Step 1: To a solution of (1S,2R)-1-[4-[4-(dimethoxymethyl)-1-piperidyl]phenyl]-2-indan-5-yl-tetralin-6-ol (70.00 mg, 140.66 μmol, 100% purity, 1 eq) in DCM (4 mL) was added TFA (1.54 g, 13.51 mmol, 1 mL, 96.0 eq). The mixture was stirred at 25° C. for 2 h. The reaction mixture was quenched by addition of saturation solution of NaHCO₃ (15 mL), extracted with EtOAc (10 mL×3). The combined organic layers were washed with brine (10 mL), dried over Na₂SO₄, filtered and concentrated under reduced pressure to yield 1-[4-[(1S,2R)-6-hydroxy-2-indan-5-yl-tetralin-1-yl]phenyl]piperidine-4-carbaldehyde (50 mg, 99.65 μmol, 70.8% yield, 90.0% purity) as a yellow solid which was used in the next step without further purification. LC-MS (ESI⁺) m/z 452.4 [M+H]⁺

Step 2: To a solution of 1-[4-[(1S,2R)-6-hydroxy-2-indan-5-yl-tetralin-1-yl]phenyl]piperidine-4-carbaldehyde (50 mg, 99.6 μmol, 90% purity, 1 eq) and (3S)-3-(1-oxo-5-piperazin-1-yl-isoindolin-2-yl)piperidine-2,6-dione (49.8 mg, 99.65 μmol, 1 eq, TsOH) in DCM (2 mL) and MeOH (2 mL) after stirred 30 min was added sodium;cyanoboranuide (31.3 mg, 498.2 μmol, 5 eq). The mixture was stirred at 25° C. for 12 h. The residue was filtered and purified by preparative HPLC (column: Boston Green ODS 150*30 mm*5 um; mobile phase: [water(FA)-ACN]; B %: 24%-54%, 12 min), followed by lyophilization to yield (3S)-3-[5-[4-[[1-[4-[(1S,2R)-6-hydroxy-2-indan-5-yl-tetralin-1-yl]phenyl]-4-piperidyl]methyl]piperazin-1-yl]-1-oxo-isoindolin-2-yl]piperidine-2,6-dione (25.3 mg, 32.69 μmol, 32.81% yield, 100% purity) as a white solid. LC-MS (ESI⁺) m/z 764.3 [M+H]⁺.

LCMS: calc. for C₄₈H₅₃N₅O₄:763.4, found: [M+H]⁺ 764.3.

HPLC: 100% purity at 220 nm.

¹H NMR (400 MHZ, CD₃OD) δ 8.28 (s, 1H), 7.66 (d, J=9.2 Hz, 1H), 7.15-7.08 (m, 2H), 6.95 (d, J=8.2 Hz, 1H), 6.71-6.62 (m, 4H), 6.60-6.55 (m, 2H), 6.51 (dd, J=2.4, 8.3 Hz, 1H), 6.32 (d, J=8.6 Hz, 2H), 5.10 (dd, J=5.2, 13.4 Hz, 1H), 4.64 (s, 4H), 4.48-4.34 (m, 2H), 4.14 (d, J=5.2 Hz, 1H), 3.57-3.46 (m, 5H), 3.25 (s, 2H), 2.97 (d, J=11.2 Hz, 4H), 2.87-2.76 (m, 4H), 2.69-2.60 (m, 4H), 2.52-2.41 (m, 1H), 2.23-2.10 (m, 2H), 2.01 (quin, J=7.2 Hz, 2H), 1.88 (d, J=12.8 Hz, 3H), 1.72 (d, J=8.2 Hz, 1H), 1.39 (d, J=11.2 Hz, 2H).

Stereochemistry at the ERBM core is arbitrarily assigned.

### Example 265. Preparation of (I-412) (3.5)-3-[5-[4-[[1-[4-[(1R,2S)-6-hydroxy-2-indan-5-yl-tetralin-1-yl]phenyl]-4-piperidyl]methyl]piperazin-1-yl]-1-oxo-isoindolin-2-yl]piperidine-2,6-dione

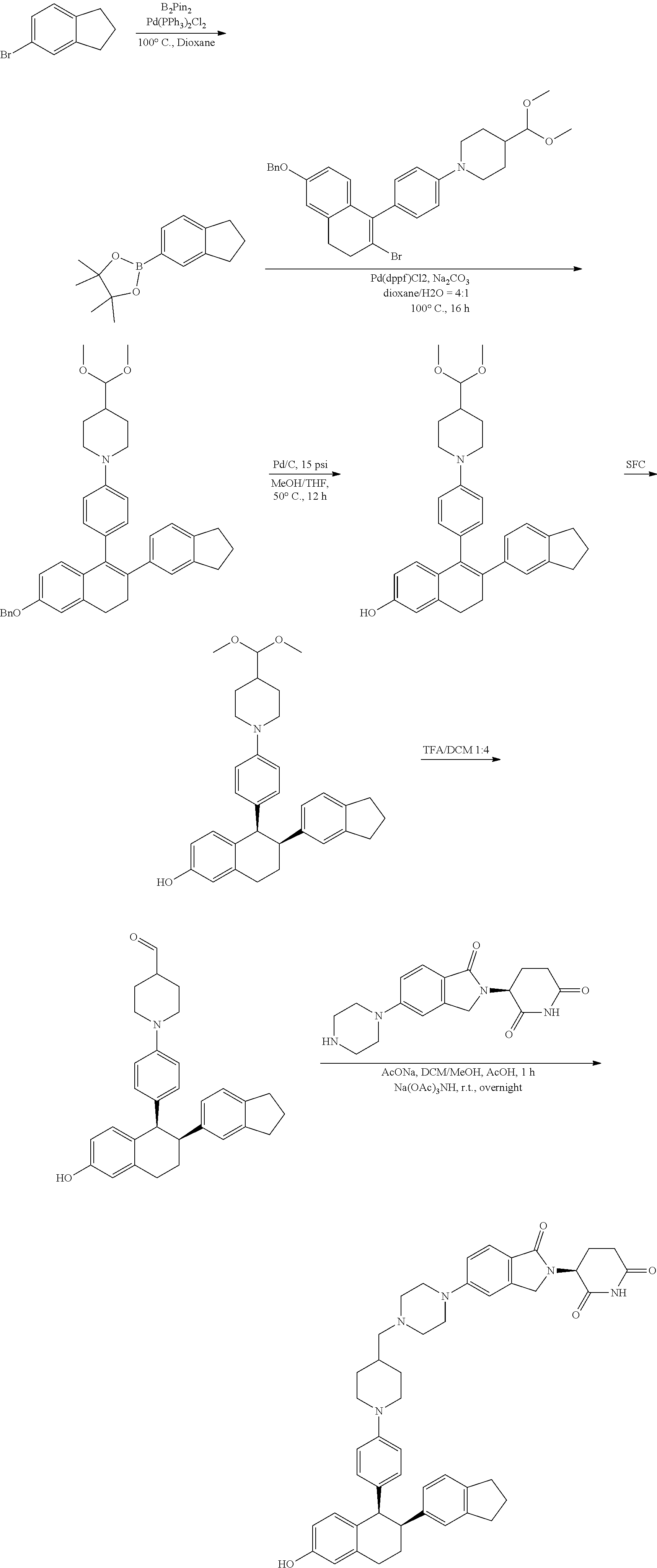

Step 1: To a solution of 5-bromoindane (700 mg, 3.5 mmol, 1 eq) and bis(pinacolato)diboron (1.8 g, 7.10 mmol, 2 eq) in DMF (30 mL) was added Pd(PPh₃)₂Cl₂ (747.9 mg, 1.07 mmol, 0.3 eq) and KOAc (1.7 g, 17.7 mmol, 5 eq). The mixture was stirred under N₂ at 100° C. for 12 h. The reaction mixture was quenched by addition of water (30 mL), extracted with EtOAc (20 mL×3). The combined organic layers were washed with brine (20 mL×3), dried over Na₂SO₄, filtered and concentrated under reduced pressure to give a residue. The residue was purified by flash silica gel chromatography (from pure PE to PE/EtOAc=5/1, TLC: PE/EtOAc=5/1, Rf=0.71) to yield 2-indan-5-yl-4,4,5,5-tetramethyl-1,3,2-dioxaborolane (400 mg, 1.39 mmol, 39.2% yield, 85% purity) as colourless oil.

Step 2: To a solution of 1-[4-(6-benzyloxy-2-bromo-3,4-dihydronaphthalen-1-yl)phenyl]-4-(dimethoxymethyl)piperidine (1.5 g, 2.71 mmol, 99% purity, 1 eq) and 2-indan-5-yl-4,4,5,5-tetramethyl-1,3,2-dioxaborolane (660.96 mg, 2.71 mmol, 1 eq) in dioxane (20 mL) and H₂O (5 mL) was added Pd(dppf)Cl₂ (198.1 mg, 270.7 mol, 0.1 eq) and Na₂CO₃ (573.90 mg, 5.4 mmol, 2 eq). The mixture was stirred under N₂ at 100° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue. The residue was purified by flash silica gel chromatography (from pure PE to PE/EtOAc=5/1, TLC: PE/EtOAc=5/1, Rf=0.54) to yield 1-[4-(6-benzyloxy-2-indan-5-yl-3,4-dihydronaphthalen-1-yl)phenyl]-4-(dimethoxymethyl)piperidine (1.4 g, 1.5 mmol, 55.6% yield, 63% purity) as yellow oil. LC-MS (ESI⁺) m/z 586.8 [M+H]⁺.

Step 3: To a solution of 1-[4-(6-benzyloxy-2-indan-5-yl-3,4-dihydronaphthalen-1-yl)phenyl]-4-(dimethoxymethyl)piperidine (1.3 g, 1.44 mmol, 65% purity, 1 eq) in MeOH (6 mL) and THE (9 mL) was added Pd/C (500 mg, 10% purity). The mixture was stirred under H₂ (15 Psi) at 25° C. for 1 h. The residue was filtered and purified by preparative HPLC (column: Welch Xtimate C18 150*25 mm*5 um; mobile phase: [water (NH₄HCO₃)-ACN]; B %: 70%-95%, 11 min), followed by lyophilization to yield a residue. The residue was purified by preparative SFC (column: DAICEL CHIRALCEL OD-H (250 mm*30 mm, 5 um); mobile phase: [0.1% NH₃H₂O MEOH]; B %: 50%-50%, min), followed by lyophilization to yield (1R,2S)-1-[4-[4-(dimethoxymethyl)-1-piperidyl]phenyl]-2-indan-5-yl-tetralin-6-ol (150 mg, 301.41 μmol, 30.00% yield, 100% purity) as a white solid. LC-MS (ESI⁺) m/z 498.3 [M+H]⁺.

Step 4: To a solution of (1R,2S)-1-[4-[4-(dimethoxymethyl)-1-piperidyl]phenyl]-2-indan-5-yl-tetralin-6-ol (60 mg, 120.56 μmol, 100% purity, 1 eq) in DCM (4 mL) was added TFA (1.32 g, 11.5 mmol, 857.12 uL, 96.0 eq). The mixture was stirred at 25° C. for 2 h. The reaction mixture was quenched by addition of saturation solution of NaHCO₃ (15 mL), extracted with EtOAc (10 mL×3). The combined organic layers were washed with brine (10 mL), dried over Na₂SO₄, filtered and concentrated under reduced pressure to yield 1-[4-[(1R,2S)-6-hydroxy-2-indan-5-yl-tetralin-1-yl]phenyl]piperidine-4-carbaldehyde (50 mg, 99.6 μmol, 82.6% yield, 90% purity) as a yellow solid which was used in the next step without further purification. LC-MS (ESI⁺) m/z 452.4 [M+H]⁺

Step 5: To a solution of 1-[4-[(1R,2S)-6-hydroxy-2-indan-5-yl-tetralin-1-yl]phenyl]piperidine-4-carbaldehyde (50 mg, 99.65 μmol, 90.0% purity, 1 eq) and (35)-3-(1-oxo-5-piperazin-1-yl-isoindolin-2-yl)piperidine-2,6-dione (49.8 mg, 99.65 μmol, 1 eq, TsOH) in DCM (2 mL) and MeOH (2 mL) after stirred 30 min was added sodium;cyanoboranuide (31.31 mg, 498.23 pol, 5 eq). The mixture was stirred at 25° C. for 12 h. The residue was filtered and purified by preparative HPLC (column: Boston Green ODS 150*30 mm*5 um; mobile phase: [water(FA)-CAN]; B %: 24%-54%, 12 min), followed by lyophilization to yield (3S)-3-15-[4-[[1-[4-[(1R,2S)-6-hydroxy-2-indan-5-yl-tetralin-1-yl]phenyl]-4-piperidyl]methyl]piperazin-1-yl]-1-oxo-isoindolin-2-yl]piperidine-2,6-dione (25.4 mg, 32.8 μmol, 32.9% yield, 100.0% purity) as a white solid. LC-MS (ESI⁺) m/z 764.3 [M+H]⁺.

LCMS: calc. for C48H53N5O4: 763.4, found: [M+H]⁺ 764.3.

HPLC: 100% purity at 220 nm.

¹H NMR (400 MHZ, CD₃OD) δ 8.24 (s, 1H), 7.67 (d, J=9.2 Hz, 1H), 7.15-7.09 (m, 2H), 6.96 (d, J=8.2 Hz, 1H), 6.71-6.63 (m, 4H), 6.60-6.55 (m, 2H), 6.51 (dd, J=2.2, 8.3 Hz, 1H), 6.33 (d, J=8.8 Hz, 2H), 5.10 (br dd, J=5.2, 13.4 Hz, 1H), 4.62 (s, 4H), 4.49-4.34 (m, 2H), 4.15 (br d, J=4.8 Hz, 1H), 3.59-3.50 (m, 5H), 3.25 (d, J=4.6 Hz, 2H), 3.06-2.99 (m, 4H), 2.88-2.77 (m, 4H), 2.75-2.64 (m, 4H), 2.46 (dq, J=4.6, 13.2 Hz, 1H), 2.24-2.11 (m, 2H), 2.07-1.99 (m, 2H), 1.88 (d, J=10.4 Hz, 3H), 1.72 (br d, J=8.0 Hz, 1H), 1.40 (q, J=11.2 Hz, 2H).

Stereochemistry at the ERBM core is arbitrarily assigned.

### Example 266. Preparation of (I-411) (3S)-3-[5-[4-[[1-[4-[(1S,2R)-6-hydroxy-2-indan-4-yl-tetralin-1-yl]phenyl]-4-piperidyl]methyl]piperazin-1-yl]-1-oxo-isoindolin-2-yl]piperidine-2,6-dione

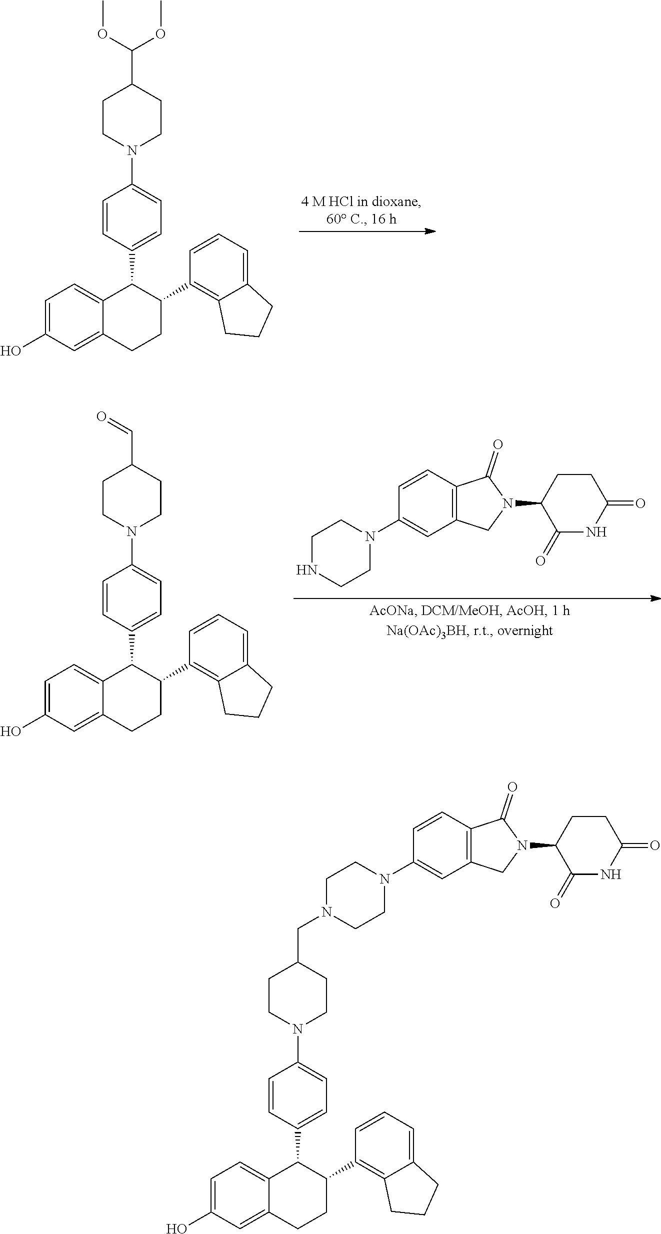 [SMILES: COC(OC)C1CCN(c2ccc([C@H]3c4ccc(O)cc4CC[C@H]3c3cccc4c3CCC4)cc2)CC1 ; O=CC1CCN(c2ccc([C@H]3c4ccc(O)cc4CC[C@H]3c3cccc4c3CCC4)cc2)CC1 ; O=C1CC[C@H](N2Cc3cc(N4CCNCC4)ccc3C2=O)C(=O)N1 ; O=C1CC[C@H](N2Cc3cc(N4CCN(CC5CCN(c6ccc([C@H]7c8ccc(O)cc8CC[C@H]7c7cccc8c7CCC8)cc6)CC5)CC4)ccc3C2=O)C(=O)N1]

Step 1: To a solution of (1S,2R)-1-[4-[4-(dimethoxymethyl)-1-piperidyl]phenyl]-2-indan-4-yl-tetralin-6-ol (30 mg, 60.28 μmol, 1 eq) in HCl/dioxane (5 mL). The mixture was stirred at 25° C. for 12 hr. The reaction mixture was concentrated under reduced pressure to remove HCl/dioxane, The crude product was used into the next step without further purification, Compound 1-[4-[(1S,2R)-6-hydroxy-2-indan-4-yl-tetralin-1-yl]phenyl]piperidine-4-carbaldehyde (30 mg, crude) was obtained as a white solid,

LC-MS (ESI⁺) m/z: 452.4 (M+H)⁺

Step 2: To a solution of 1-[4-[(1S,2R)-6-hydroxy-2-indan-4-yl-tetralin-1-yl]phenyl]piperidine-4-carbaldehyde (30 mg, 66.43 μmol, 1 eq) and (3S)-3-(1-oxo-5-piperazin-1-yl-isoindolin-2-yl)piperidine-2,6-dione (33 mg, 99.65 μmol, 1.5 eq) in DCM (2 mL) and MeOH (2 mL) was added acetic acid (18 mg, 66.43 μmol, 17.47 uL, 1 eq), sodium acetate (16 mg, 199.3 μmol, 3 eq). The mixture was stirred at 25° C. for 1 hr, and then was added NaBH(OAc) 3 (21 mg, 99.65 μmol, 1.5 eq). The mixture was stirred at 25° C. for 15 hr. The reaction mixture was concentrated under reduced pressure to remove DCM and MeOH. The residue was purified by prep-HPLC (column: Welch Ultimate C18 150*25 mm*5 um; mobile phase: [water(FA)-ACN]; B %: 26%-56%, 10 min). Compound (3S)-3-[5-[4-[[1-[4-[(1S,2R)-6-hydroxy-2-indan-4-yl-tetralin-1-yl]phenyl]-4-piperidyl]methyl]piperazin-1-yl]-1-oxo-isoindolin-2-yl]piperidine-2,6-dione (14 mg, 17.46 μmol, 26.28% yield, 95.25% purity) was obtained as a white solid.

LC-MS (ESI+) m/z: 764.2 (M+H)⁺

HPLC: 94.910% purity at 220 nm

SFC: Rₜ: 3.058 min; Area, 94.997%; Stereochemistry at the ERBM core is arbitrarily assigned.

¹H NMR (400 MHZ, CHLOROFORM-d) δ=7.95-7.88 (m, 1H), 7.78-7.70 (m, 1H), 7.07-6.95 (m, 2H), 6.90-6.77 (m, 3H), 6.74-6.70 (m, 1H), 6.63-6.53 (m, 3H), 6.29-6.23 (m, 2H), 6.18-6.11 (m, 1H), 5.20 (dd, J=4.9, 13.1 Hz, 1H), 4.45-4.40 (m, 1H), 4.30-4.21 (m, 2H), 3.61-3.51 (m, 2H), 3.44-3.32 (m, 4H), 3.06-2.82 (m, 9H), 2.68-2.56 (m, 5H), 2.34 (br dd, J=5.0, 12.4 Hz, 2H), 2.26-1.96 (m, 6H), 1.94-1.81 (m, 3H), 1.42-1.31 (m, 3H).

### Example 267. Preparation of (I-410) (3S)-3-[5-[4-[[1-[4-[(1R,2S)-6-hydroxy-2-indan-4-yl-tetralin-1-yl]phenyl]-4-piperidyl]methyl]piperazin-1-yl]-1-oxo-isoindolin-2-yl]piperidine-2,6-dione

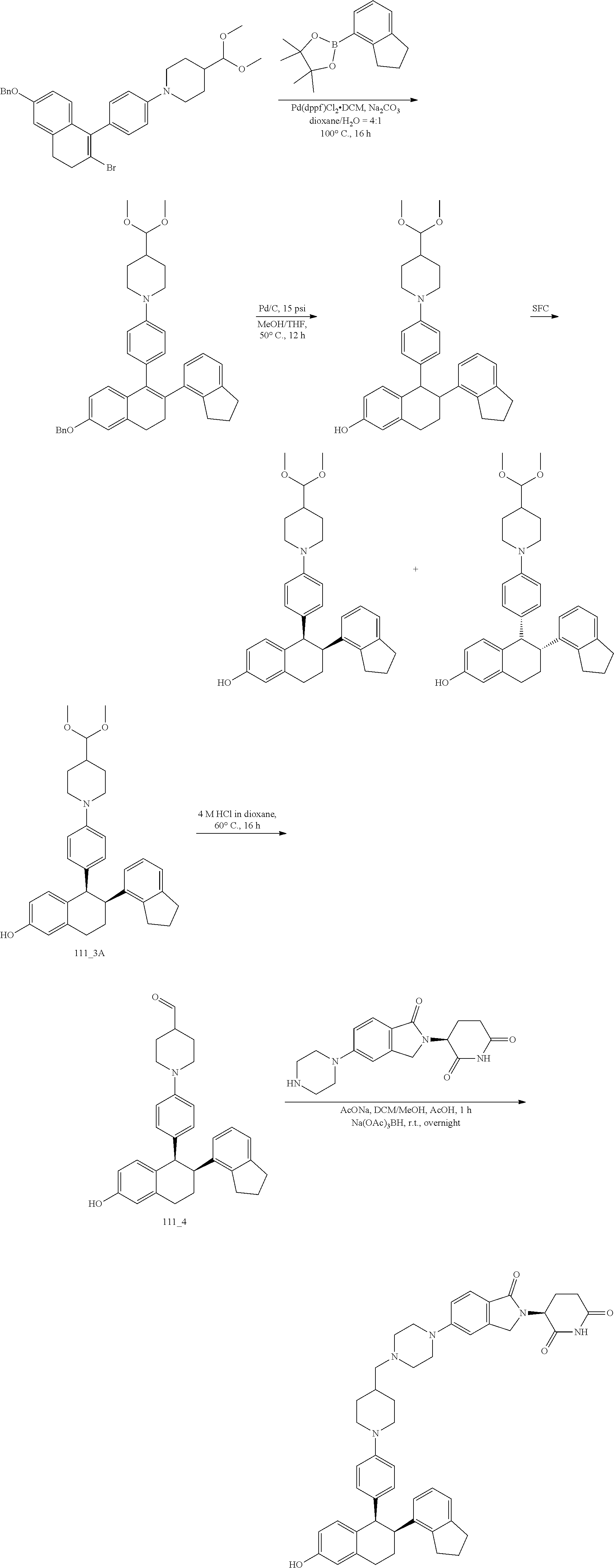

Step 1: A mixture of 1-[4-(6-benzyloxy-2-bromo-3,4-dihydronaphthalen-1-yl)phenyl]-4-(dimethoxymethyl)piperidine (500 mg, 911.56 μmol, 1 eq), 2-indan-4-yl-4,4,5,5-tetramethyl-1,3,2-dioxaborolane (289 mg, 1.19 mmol, 1.3 eq), Pd(dppf)Cl₂·DCM (372 mg, 455.78 μmol, 0.5 eq), Na₂CO₃ (290 mg, 2.73 mmol, 3 eq) in dioxane (4 mL) and H₂O (1 mL) was degassed and purged with N₂ for 3 times, and then the mixture was stirred at 100° C. for 16 hr under N₂ atmosphere. The reaction mixture was concentrated under reduced pressure to remove dioxane and H2O, The residue was purified by flash silica gel chromatography (ISCO®; 12 g SepaFlash® Silica Flash Column, Eluent of 0˜30% Ethyl acetate/Petroleum ether @ 40 mL/min). Compound 1-[4-(6-benzyloxy-2-indan-4-yl-3,4-dihydronaphthalen-1-yl)phenyl]-4-(dimethoxymethyl)piperidine (300 mg, 394.7 μmol, 43.3% yield, 77.1% purity) was obtained as a yellow solid.

LC-MS (ESI⁺) m/z: 586.3 (M+H)⁺

Step 2: A mixture of 1-[4-(6-benzyloxy-2-indan-4-yl-3,4-dihydronaphthalen-1-yl)phenyl]-4-(dimethoxymethyl)piperidine (300.00 mg, 512.14 μmol, 1 eq), Pd/C (200 mg, 2.56 mmol, 10% purity, 5 eq) in MeOH (2 mL) and THF (2 mL) was degassed and purged with H₂ for 3 times, and then the mixture was stirred at 50° C. for 12 hr under H₂ atmosphere. The reaction mixture was filtered and the filter cake was rinsed with 50 mL of MeOH, The filter was collected, dried in vacuo to give a white solid, The residue was purified by flash silica gel chromatography (ISCO®; 12 g SepaFlash® Silica Flash Column, Eluent of 0˜30% Ethyl acetate/Petroleum ether @ 40 mL/min) to give desired compound as a colorless oil, which was further separated by SFC (column: Welch Ultimate C18 150*25 mm*5 um; mobile phase: [water(FA)-ACN]; B %: 40%-70%, 10 min) and (column: Welch Ultimate C18 150*25 mm*5 um; mobile phase: [water(FA)-ACN]; B %: 38%-68%, 10 min).

Compound (1R,2S)-1-[4-[4-(dimethoxymethyl)-1-piperidyl]phenyl]-2-indan-4-yl-tetralin-6-ol (73 mg, 140.9 μmol, 27.5% yield, 96.0% purity) was obtained as a white solid.

LC-MS (ESI+) m/z: 498.5 (M+H)⁺

HPLC: 95.454% purity at 220 nm

SFC: Rₜ: 1.760 min; Area, 100%;

¹H NMR (400 MHZ, CHLOROFORM-d) δ=7.06-7.02 (m, 1H), 6.87-6.79 (m, 2H), 6.73-6.69 (m, 1H), 6.62-6.54 (m, 3H), 6.29-6.21 (m, 2H), 6.17-6.12 (m, 1H), 4.25-4.19 (m, 1H), 4.09-4.02 (m, 1H), 3.63 (s, 2H), 3.40-3.34 (m, 6H), 3.06-2.79 (m, 6H), 2.62-2.46 (m, 2H), 2.24-2.00 (m, 3H), 1.84-1.73 (m, 4H), 1.49-1.28 (m, 4H)

Compound (1S,2R)-1-[4-[4-(dimethoxymethyl)-1-piperidyl]phenyl]-2-indan-4-yl-tetralin-6-ol (80 mg, 112.09 μmol, 21.89% yield, 69.730% purity) was obtained as a white solid.

LC-MS (ESI+) m/z: 498.5 (M+H)⁺

HPLC: 97.288% purity at 220 nm

SFC: Rₜ: 2.045 min; Area, 99.894%; method: OJ-3-MeOH (DEA)-5-40-3 mL-35T.

¹H NMR (400 MHZ, CHLOROFORM-d) δ=7.07-7.01 (m, 1H), 6.87-6.78 (m, 2H), 6.73-6.69 (m, 1H), 6.64-6.54 (m, 3H), 6.29-6.22 (m, 2H), 6.17-6.12 (m, 1H), 4.26-4.21 (m, 1H), 4.09-4.04 (m, 1H), 3.61-3.51 (m, 2H), 3.38-3.34 (m, 6H), 2.85 (br d, J=6.9 Hz, 6H), 2.65-2.46 (m, 2H), 2.23-2.01 (m, 3H), 1.86-1.68 (m, 6H), 1.49-1.25 (m, 5H).

Step 3: To a solution of (1R,2S)-1-[4-[4-(dimethoxymethyl)-1-piperidyl]phenyl]-2-indan-4-yl-tetralin-6-ol (30 mg, 60.28 μmol, 1 eq) in HCl/dioxane (5 mL). The mixture was stirred at 25° C. for 16 hr. The reaction mixture was concentrated under reduced pressure to remove HCl/dioxane, The crude product was used into the next step without further purification, Compound 1-[4-[(1R,2S)-6-hydroxy-2-indan-4-yl-tetralin-1-yl]phenyl]piperidine-4-carbaldehyde (30 mg, crude) was obtained as a white solid.

LC-MS (ESI⁺) m/z: 452.5 (M+H)⁺.

Step 4: To a solution of 1-[4-[(1R,2S)-6-hydroxy-2-indan-4-yl-tetralin-1-yl]phenyl]piperidine-4-carbaldehyde (30 mg, 66.43 μmol, 1 eq) and (3S)-3-(1-oxo-5-piperazin-1-yl-isoindolin-2-yl)piperidine-2,6-dione (33 mg, 99.65 μmol, 1.5 eq) in DCM (2 mL) and MeOH (2 mL) was added acetic acid (18 mg, 66.43 μmol, 17.47 uL, 1 eq) and sodium acetate (16, 199.29 μmol, 3 eq), The mixture was stirred at 25° C. for 1 hr and then was added NaBH(OAc)₃ (21 mg, 99.65 μmol, 1.5 eq), The mixture was stirred at 25° C. for 15 hr. The reaction mixture was concentrated under reduced pressure to remove DCM and MeOH. The residue was purified by prep-HPLC (column: Phenomenexluna C18 150*25 mm*10 um; mobile phase: [water(FA)-ACN]; B %:

24%-54%, 8 min). Compound (3S)-3-[5-[4-[[1-[4-[(1R,2S)-6-hydroxy-2-indan-4-yl-tetralin-1-yl]phenyl]-4-piperidyl]methyl]piperazin-1-yl]-1-oxo-isoindolin-2-yl]piperidine-2,6-dione (7.5 mg, 9.66 μmol, 14.54% yield, 98.42% purity) was obtained as a white solid.

LC-MS (ESI+) m/z: 764.2 (M+H)⁺

HPLC: 98.375% purity at 220 nm

SFC: Rₜ: 1.297 min; Area, 96.154%; Stereochemistry at the ERBM core is arbitrarily assigned ¹H NMR (400 MHZ, CHLOROFORM-d) δ=7.86-7.80 (m, 1H), 7.69-7.63 (m, 1H), 6.98-6.89 (m, 2H), 6.82-6.71 (m, 3H), 6.63 (d, J=2.4 Hz, 1H), 6.55-6.46 (m, 3H), 6.21-6.15 (m, 2H), 6.09-6.04 (m, 1H), 5.16-5.09 (m, 1H), 4.37-4.30 (m, 1H), 4.23-4.13 (m, 2H), 3.52-3.44 (m, 2H), 3.36-3.25 (m, 4H), 2.97-2.71 (m, 9H), 2.60-2.47 (m, 5H), 2.25 (br dd, J=5.1, 12.6 Hz, 2H), 2.17-1.91 (m, 6H), 1.82-1.75 (m, 3H), 1.36-1.24 (m, 3H).

### Example 268. Preparation of (I-409) (S)-3-(5-(4-((4-(4-((1S,2S)-6-hydroxy-2-phenyl-1,2,3,4-tetrahydronaphthalen-1-yl)-1H-pyrazol-1-yl)piperidin-1-yl)methyl)piperidin-1-yl)-1-oxoisoindolin-2-yl)piperidine-2,6-dione and (I-48) (R)-3-(5-(4-((4-(4-((1S,2S)-6-hydroxy-2-phenyl-1,2,3,4-tetrahydronaphthalen-1-yl)-1H-pyrazol-1-yl)piperidin-1-yl)methyl)piperidin-1-yl)-1-oxoisoindolin-2-yl)piperidine-2,6-dione

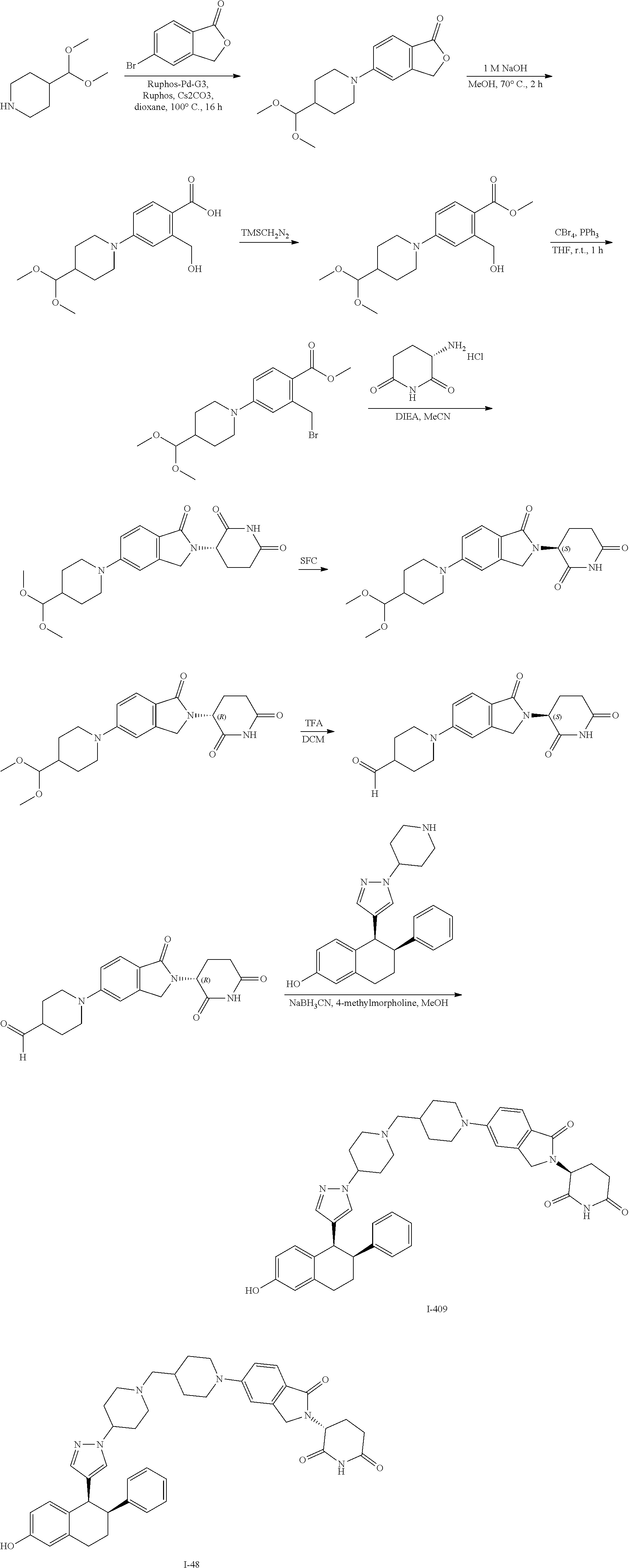

Step 1: A mixture of 4-(dimethoxymethyl)piperidine (2.0 g, 1 Eq, 9.4 mmol), 5-bromoisobenzofuran-1 (3H)-one (1.5 g, 1 Eq, 9.4 mmol), Ruphos-Pd-G3 (0.55 g, 0.07 Eq, 0.66 mmol) and Cs₂CO₃ (6.1 g, 1.5 mL, 2 Eq, 19 mmol) in 1,4-Dioxane (40 mL). The mixture was stirred at 100° C. for 16 hour. The mixture was concentrated in reduced pressure. The residue was poured into water (40 mL). The aqueous phase was extracted with ethyl acetate (80 mL*3). The combined organic phase was washed with brine (20 mL), dried with Na₂SO₄, filtered and concentrated in vacuum. The residue was purified by silica gel chromatography

(Petroleum ether/Ethyl acetate=10/1 to 3/1) LCMS indicated no desired compound. 5-(4-(dimethoxymethyl)piperidin-1-yl)isobenzofuran-1 (3H)-one (1.5 g, 5.1 μmol, 55%) was obtained as oil.

LC-MS (ESI+) m/z: 292.2 (M+H)+.

Step 2: To a solution of 5-(4-(dimethoxymethyl)piperidin-1-yl)isobenzofuran-1 (3H)-one (1.81 g, 1 Eq, 6.21 mmol), NaOH (1.8 g, 7.1 Eq, 44.1 mmol) in MeOH (9.05 mL), H₂O (9.05 mL) and THF (18 mL). The mixture was stirred at 25° C. for 1 hour. LCMS showed that the starting material was consumed and the desired product was detected. Concentrated aqueous HCl was added to the mixture until a precipitate formed, which was collected by filtration to give the 4-(4-(dimethoxymethyl)piperidin-1-yl)-2-(hydroxymethyl)benzoic acid (1.5 g, 4.8 mmol, 78%).

Step 3: To a solution of 4-(4-(dimethoxymethyl)piperidin-1-yl)-2-(hydroxymethyl)benzoic acid (2.0 g, 1.0 Eq, 6.5 mmol) and TMSCH₂N₂ (2.8 g, 12 mL, 2 molar, 3.8 Eq, 25 mmol) in MeOH (12.5 mL) and EtOAc (12.5 mL). The mixture was stirred at −10° C. for 0.25 hour. LCMS showed that the starting material was consumed and the desired product was detected. The residue was poured into water (25 mL). The aqueous phase was extracted with EtOAc (50 mL*3). The combined organic phase was washed with brine (10 mL*3), dried with anhydrous Na₂SO₄, filtered and concentrated in vacuum to give a residue. The reaction mixture was filtered under reduced pressure to give a compound. methyl 4-(4-(dimethoxymethyl)piperidin-1-yl)-2-(hydroxymethyl)benzoate (1.8 g, 5.6 mmol, 86%) was obtained as a solid. LC-MS (ESI+) m/z: 324.2 (M+H)+.

Step 4: A mixture of methyl 4-(4-(dimethoxymethyl)piperidin-1-yl)-2-(hydroxymethyl)benzoate (765.0 mg, 1 Eq, 2.366 mmol), CBr₄ (1.177 g, 372 μL, 1.5 Eq, 3.548 mmol) and PPh3 (930.7 mg, 792.1 uL, 1.5 Eq, 3.548 mmol) in THF (9 mL). The mixture was stirred at 25° C. for 1 hour. LCMS showed that the starting material was consumed and the desired product was detected. The reaction mixture was filtered under reduced pressure to give a residue. The residue was poured into H₂O (10 mL). The aqueous phase was extracted with ethyl acetate (20 mL*3). The combined organic phase was washed with brine (10 mL*3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=3:1) to afford methyl 2-(bromomethyl)-4-(4-(dimethoxymethyl)piperidin-1-yl)benzoate (603 mg, 1.56 mmol, 66.0%) as white solid. LC-MS (ESI+) m/z: 387.9 (M+H)+.

Step 5: A mixture of (S)-3-aminopiperidine-2,6-dione hydrochloride (144.7 mg, 1.2 Eq, 879.1 μmol), methyl 2-(bromomethyl)-4-(4-(dimethoxymethyl)piperidin-1-yl)benzoate (283 mg, 1 Eq, 732.6 μmol) and DIEA (30 mg, 41 μL, 3 Eq, 0.23 mmol) in MeCN (8.5 mL). The mixture was stirred at 75° C. for 48 hour. LCMS showed that the starting material was consumed and the desired product was detected. The residue was poured into water (10 mL). The aqueous phase was extracted with DCM (10 mL*3). The combined organic phase was washed with brine (5 mL*3), dried with anhydrous Na₂SO₄, filtered and concentrated in vacuum. The residue was purified by silica gel chromatography (column height: 250 mm, diameter: 100 mm, 100-200 mesh silica gel, DCM/MeOH=10:1) to afford (S)-3-(5-(4-(dimethoxymethyl)piperidin-1-yl)-1-oxoisoindolin-2-yl)piperidine-2,6-dione (200 mg, 498 μmol, 68.0%) as solid. LC-MS (ESI+) m/z: 402.3 (M+H)+.

Step 6: (S)-3-(5-(4-(dimethoxymethyl)piperidin-1-yl)-1-oxoisoindolin-2-yl)piperidine-2,6-dione (369 mg) was separated by chiral SFC(Column DAICEL CHIRALPAK AS (250 mm*30 mm, 10 um) Condition 0.1% NH₃H₂O ETOH Begin B 30% End B 30%), 0.1% NH₃H₂O ETOH) (S)-3-(5-(4-(dimethoxymethyl)piperidin-1-yl)-1-oxoisoindolin-2-yl)piperidine-2,6-dione (100 mg, 235.3 μmol, 49%), (R)-3-(5-(4-(dimethoxymethyl)piperidin-1-yl)-1-oxoisoindolin-2-yl)piperidine-2,6-dione (100 mg, 225.9 μmol, 48%).

Step 7: (S)-1-(2-(2,6-dioxopiperidin-3-yl)-1-oxoisoindolin-5-yl)piperidine-4-carbaldehyde: A mixture of (S)-3-(5-(4-(dimethoxymethyl)piperidin-1-yl)-1-oxoisoindolin-2-yl)piperidine-2,6-dione (85 mg, 1 Eq, 0.21 mmol), and TFA (0.37 g, 0.25 mL, 15 Eq, 3.2 mmol) in DCM (1 mL) in DCM (1 mL) and then the mixture was stirred at 25° C. for 1 hour. LCMS showed that the starting material was consumed and the desired product was detected. The reaction mixture was filtered under reduced pressure to give a residue. (S)-1-(2-(2,6-dioxopiperidin-3-yl)-1-oxoisoindolin-5-yl)piperidine-4-carbaldehyde (50 mg, 0.14 mmol, 66%) was obtained as solid.

(R)-1-(2-(2,6-dioxopiperidin-3-yl)-1-oxoisoindolin-5-yl)piperidine-4-carbaldehyde: A mixture of (R)-3-(5-(4-(dimethoxymethyl)piperidin-1-yl)-1-oxoisoindolin-2-yl)piperidine-2,6-dione (100 mg, 1 Eq. 272 μmol), and TFA (1 g, 1 mL, 5e+1 Eq, 0.01 mol) (935 mg, 841 μL, 20 Eq, 5.43 mmol) in DCM (4 mL) and then the mixture was stirred at 25° C. for 1 hour. LCMS showed that the starting material was consumed and the desired product was detected. The reaction mixture was filtered under reduced pressure to give a residue. (R)-1-(2-(2,6-dioxopiperidin-3-yl)-1-oxoisoindolin-5-yl)piperidine-4-carbaldehyde (90 mg, 0.25 mmol, 93%) was obtained as solid.

Step 8: A mixture of (5S,6S)-6-phenyl-5-(1-(piperidin-4-yl)-1H-pyrazol-4-yl)-5,6,7,8-tetrahydronaphthalen-2-ol (89.3 mg, 1 Eq, 239 μmol), (S)-1-(2-(2,6-dioxopiperidin-3-yl)-1-oxoisoindolin-5-yl)piperidine-4-carbaldehyde (85.0 mg, 1 Eq, 239 μmol) and NaBH₃CN (105 mg, 7 Eq, 1.67 mmol) in DCM (1 mL) and MeOH (1 mL). The mixture was stirred at 25° C. for 1 hour. LCMS showed that the starting material was consumed and the desired product was detected. The reaction mixture was filtered under reduced pressure to give a residue. The residue was purified by prep-HPLC (column: Boston Green ODS 150*30 mm*5 um; mobile phase: [water(FA)-ACN]; B %: 21%-41%, 10 min) to give (S)-3-(5-(4-((4-(4-((1S,2S)-6-hydroxy-2-phenyl-1,2,3,4-tetrahydronaphthalen-1-yl)-1H-pyrazol-1-yl)piperidin-1-yl)methyl)piperidin-1-yl)-1-oxoisoindolin-2-yl)piperidine-2,6-dione (20 mg, 28 μmol, 12%) as white solid.

LC-MS (ESI+) m/z: 713.4 (M+H)+.

HPLC: 98.097% purity at 220 nm.

¹H NMR (400 MHZ, DMSO-d₆) δ=10.95 (s, 1H), 9.11 (s, 1H), 7.49 (d, J=8.8 Hz, 1H), 7.25-7.15 (m, 3H), 7.06-7.01 (m, 2H), 6.94 (br d, J=7.2 Hz, 2H), 6.76 (d, J=8.3 Hz, 1H), 6.56 (s, 1H), 6.52-6.45 (m, 2H), 6.33 (s, 1H), 5.07-5.01 (m, 1H), 4.34-4.28 (m, 1H), 4.22-4.16 (m, 1H), 4.12 (br d, J=4.5 Hz, 1H), 3.89-3.87 (m, 1H), 3.25-3.16 (m, 2H), 2.96-2.75 (m, 8H), 2.04-1.90 (m, 3H), 1.82-1.70 (m, 8H), 1.26-1.08 (m, 3H), 0.05-−0.25 (m, 4H).

Step 9: A mixture of (5S,6S)-6-phenyl-5-(1-(piperidin-4-yl)-1H-pyrazol-4-yl)-5,6,7,8-tetrahydronaphthalen-2-ol (105.1 mg, 1 Eq, 281.4 μmol), (R)-1-(2-(2,6-dioxopiperidin-3-yl)-1-oxoisoindolin-5-yl)piperidine-4-carbaldehyde (100.0 mg, 1 Eq, 281.4 μmol) and NaBH₃CN (123.8 mg, 7 Eq, 1.970 mmol) in DCM (1 mL) and MeOH (1 mL). The mixture was stirred at 25° C. for 1 hour. LCMS showed that the starting material was consumed and the desired product was detected. The reaction mixture was filtered under reduced pressure to give a residue. The residue was purified by prep-HPLC (column: Boston Green ODS 150*30 mm+5 um; mobile phase: [water(FA)-ACN]; B %: 21%-41%, 10 min) to give (R)-3-(5-(4-((4-(4-((1S,2S)-6-hydroxy-2-phenyl-1,2,3,4-tetrahydronaphthalen-1-yl)-1H-pyrazol-1-yl)piperidin-1-yl)methyl)piperidin-1-yl)-1-oxoisoindolin-2-yl)piperidine-2,6-dione (34.2 mg, 48 μmol, 17%) as white solid.

LC-MS (ESI+) m/z: 713.4 (M+H)⁺.

HPLC: 96.59% purity at 220 nm.

¹H NMR (400 MHZ, DMSO-d₆) δ=10.95 (s, 1H), 9.11 (s, 1H), 8.13 (s, 1H), 7.51 (d, J=8.9 Hz, 1H), 7.24-7.15 (m, 3H), 7.09-7.04 (m, 2H), 6.95 (br d, J=7.2 Hz, 2H), 6.76 (d, J=8.5 Hz, 1H), 6.58-6.49 (m, 3H), 6.34 (s, 1H), 5.04 (dd, J=5.1, 13.3 Hz, 1H), 4.34-4.29 (m, 1H), 4.21-4.13 (m, 2H), 3.91-3.86 (m, 2H), 3.21 (br d, J=4.9 Hz, 2H), 2.95-2.79 (m, 9H), 2.41-2.29 (m, 5H), 2.02-1.91 (m, 5H), 1.78 (br d, J=12.0 Hz, 4H), 1.28-1.18 (m, 2H). The LBM stereochemistry was arbitrarily assigned.

### Example 269. Preparation of (I-41) 3-[4-[4-[[1-[4-[(1S,2R)-6-hydroxy-2-phenyl-tetralin-1-yl]phenyl]-4-piperidyl]piperazin-1-yl]phenyl]piperidine-2, 6-dione

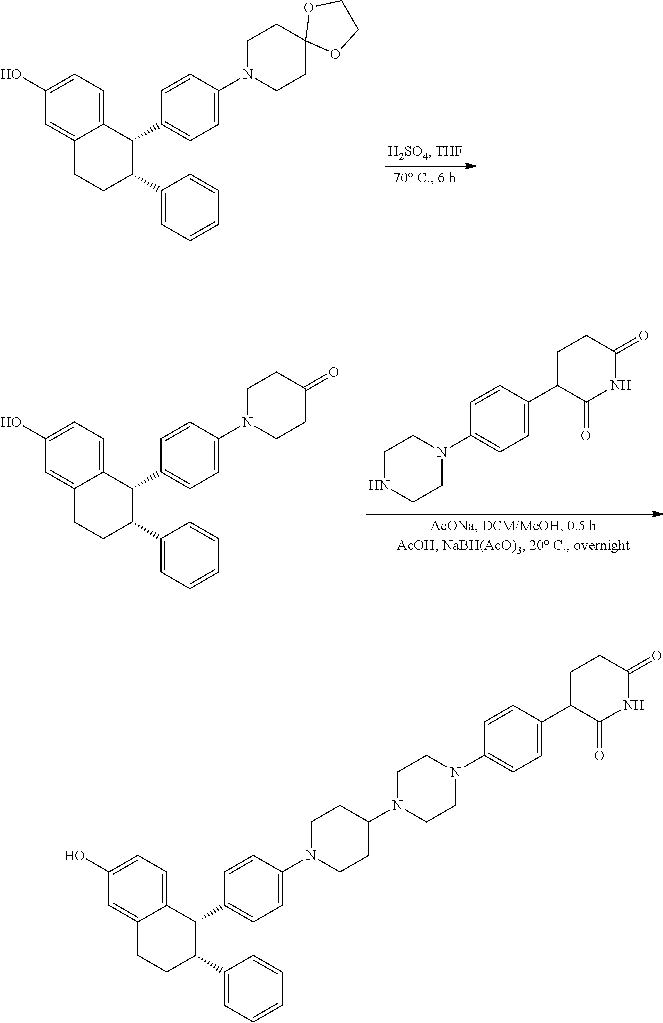 [SMILES: Oc1ccc2c(c1)CC[C@@H](c1ccccc1)[C@H]2c1ccc(N2CCC3(CC2)OCCO3)cc1 ; O=C1CCN(c2ccc([C@H]3c4ccc(O)cc4CC[C@H]3c3ccccc3)cc2)CC1 ; O=C1CCC(c2ccc(N3CCNCC3)cc2)C(=O)N1 ; O=C1CCC(c2ccc(N3CCN(C4CCN(c5ccc([C@H]6c7ccc(O)cc7CC[C@H]6c6ccccc6)cc5)CC4)CC3)cc2)C(=O)N1]

Step 1: To a solution of ((5S,6R)-5-(4-(1,4-dioxa-8-azaspiro[4.5]decan-8-yl)phenyl)-6-phenyl-5,6,7,8-tetrahydronaphthalen-2-ol (200 mg, 1 Eq, 453 μmol)) in THF (15 mL) was added Sulfuric acid (25 g, 15 mL, 10% Wt, 56 Eq, 25 mmol). The mixture was stirred at 70° C. for 6 h.

TLC (petroleum ether:ethyl acetate=1:1,) indicated Reactant 1 was consumed completely and one new spots formed. The reaction was concentrated under reduced pressure. The residue was purified by flash silica gel chromatography (ISCO®; 40 g SepaFlash® Silica Flash Column, Eluent of 0˜100% Ethyl acetate/Petroleum ether gradient @ 80 mL/min) to give the product of give 1-(4-((1S,2R)-6-hydroxy-2-phenyl-1,2,3,4-tetrahydronaphthalen-1-yl)phenyl)piperidin-4-one (200 mg, 503 μmol,) as a light yellow solid.

Step 2: To 1-(4-((1S,2R)-6-hydroxy-2-phenyl-1,2,3,4-tetrahydronaphthalen-1-yl)phenyl)piperidin-4-one (40 mg, 1 Eq, 0.10 mmol), and 3-(4-(piperazin-1-yl)phenyl)piperidine-2,6-dione (41 mg, 1.5 Eq, 0.15 mmol) and Titanium tetraisopropoxide (57 mg, 60 μL, 2 Eq, 0.20 mmol) in DCM (1 mL) and MeOH (1 mL) at 0° C. The resulting mixture was stirred for 3 hours at 20° C. Sodium cyanoborohydride (25 mg, 24 μL, 4 Eq, 0.40 mmol) was added to the mixture at 0° C. The resulting mixture was stirred for 16 h at 20° C. The yellow solid was purified by Prep.HPLC (column: Column Phenomenex C18 75*30 mm*3 um Condition: [water(FA)-ACN]; B %: 20%-60%, 8 min) the pure fractions were collected and the solvent was evaporated under vacuum. The aqueous layer was lyophilized to dryness to give 3-[4-[4-[[1-[4-[(1S, 2R)-6-hydroxy-2-phenyl-tetralin-1-yl]phenyl]-4-piperidyl]piperazin-1-yl]phenyl]piperidine-2, 6-dione (13.1 mg, 19.78 μmol, 32.37% yield, 98.85% purity) was obtained as a white solid.

LCMS: calc. for C₄₂H₄₆N₄O₃:654.36, found: [M+H]⁺ 655.2.

HPLC: 98.853% purity at 220 nm.

¹H NMR (400 MHZ, DMSO-d₆)₂=10.79 (s, 1H), 9.13 (br s, 1H), 8.15 (s, 1H), 7.21-7.02 (m, 5H), 6.95-6.79 (m, 4H), 6.67-6.46 (m, 5H), 6.29-6.15 (m, J=8.3 Hz, 2H), 4.14 (br d, J=4.3 Hz, 1H), 3.77-3.59 (m, 3H), 3.32-3.17 (m, 5H), 3.03-2.76 (m, 6H), 2.71-2.57 (m, 3H), 2.17-1.90 (m, 5H), 1.75-1.49 (m, 3H), 1.33-1.21 (m, 1H)

SFC: retention time, 5.081 min, 7.092 min; Area, 50.26%, 49.74%; Stereochemistry was arbitrarily assigned.

### Example 270. Preparation of Additional Compounds from Table 2

The following compounds were synthesized using similar synthetic schemes described in the Examples described herein.

| Compound | Analytical data |
|---|---|
| II-1 | LCMS: calc. for C₄₆H₅₁N₅O₄: 737.4, found: [M + H]⁺ 738.4. |
| | ¹H NMR (400 MHz, DMSO-d₆) δ = 10.95 (s, 1H), 9.11 (s, 1H), 8.14 (s, |
| | 0.33H), 7.56-7.45 (m, 1H), 7.20-7.01 (m, 5H), 6.84 (br d, J = 6.8 Hz, 2H), |
| | 6.69-6.45 (m, 5H), 6.20 (d, J = 8.5 Hz, 2H), 5.05 (dd, J = 4.9, 13.2 Hz, 1H), |
| | 4.33 (d, J = 17.1 Hz, 1H), 4.27-4.08 (m, 2H), 3.69-3.46 (m, 4H), 3.31 (br s, |
| | 7H), 3.02-2.84 (m, 4H), 2.61 (br s, 1H), 2.43-2.20 (m, 2H), 2.17-1.90 (m, |
| | 3H), 1.84 (br d, J = 12.0 Hz, 1H), 1.67 (br d, J = 12.8 Hz, 3H), 1.27-1.00 (m, |
| | 5H) |
| II-2 | LCMS: calc. for C₄₆H₅₁N₅O₄: 737.39, found: [M + H]⁺ 738.4. |
| | ¹H NMR (400 MHz, DMSO-d₆): δ = 10.96 (s, 1H), 9.12 (s, 1H), 7.52 (br s, |
| | 1H), 7.19-7.02 (m, 5H), 6.84 (br d, J = 6.8 Hz, 2H), 6.66-6.47 (m, 5H), 6.21 |
| | (br d, J = 8.3 Hz, 2H), 5.05 (br d, J = 8.5 Hz, 1H), 4.37-4.10 (m, 3H), 3.56 (br s, |
| | 3H), 3.33-3.23 (m, 3H), 3.10-2.79 (m, 6H), 2.62-2.55 (m, 3H), 2.39 (br s, |
| | 2H), 2.23 (br s, 1H), 2.10 (br dd, J = 6.1, 12.2 Hz, 1H), 1.97 (br d, J = 5.8 Hz, |
| | 2H), 1.85 (br d, J = 12.0 Hz, 1H), 1.76-1.54 (m, 3H), 1.37 (br s, 1H), 1.29- |
| | 1.15 (m, 2H), 1.05 (br s, 2H) |
| II-3 | LCMS: calc. for C₄₆H₅₁N₅O₄: 737.9, found: [M + H]⁺ 738.1. |
| | ¹H NMR (400 MHz, METHANOL-d₄): δ 8.30 (s, 0.19 H), 7.65 (d, J = 8.5 Hz, |
| | 1H), 7.18-7.01 (m, 5H), 6.87-6.79 (m, 2H), 6.73-6.63 (m, 4H), 6.53 (dd, |
| | J = 2.4, 8.4 Hz, 1H), 6.34 (d, J = 8.6 Hz, 2H), 5.12 (dd, J = 5.1, 13.2 Hz, 1H), 4.48- |
| | 4.34 (m, 2H), 4.21 (br d, J = 5.0 Hz, 2H), 3.55 (br d, J = 9.9 Hz, 4H), 3.28- |
| | 3.15 (m, 1H), 3.08-2.99 (m, 3H), 2.96-2.87 (m, 2H), 2.96-2.87 (m, 1H), |
| | 2.83-2.73 (m, 1H), 2.62 (br t, J = 12.7 Hz, 3H), 2.48 (br dd, J = 4.9, 13.2 Hz, |
| | 1H), 2.34 (br s, 3H), 2.27-2.10 (m, 2H), 1.91 (br s, 2H), 1.77 (br s, 2H), 1.43- |
| | 1.30 (m, 3H) |
| II-4 | LCMS: calc. for C₄₆H₅₁N₅O₄: 737.9, found: [M + H]⁺ 738.1. |
| | ¹H NMR (400 MHz, METHANOL-d₄): δ 8.29 (s, 1H), 7.63 (d, J = 8.5 Hz, 1H), |
| | 7.16-7.02 (m, 5H), 6.81 (br d, J = 7.7 Hz, 2H), 6.72-6.62 (m, 4H), 6.57-6.48 |
| | (m, 1H), 6.32 (d, J = 8.5 Hz, 2H), 5.10 (dd, J = 5.2, 13.6 Hz, 1H), 4.50-4.31 (m, |
| | 2H), 4.19 (br d, J = 4.6 Hz, 2H), 3.61-3.46 (m, 4H), 3.26-3.12 (m, 1H), 3.07- |
| | 2.95 (m, 3H), 2.93-2.72 (m, 3H), 2.60 (br t, J = 12.3 Hz, 2H), 2.46 (br dd, |
| | J = 4.9, 13.2 Hz, 2H), 2.38-2.12 (m, 4H), 1.88 (br s, 2H), 1.76 (br d, J = 10.6 |
| | Hz, 2H), 1.42-1.27 (m, 2H), 1.18 (d, J = 6.4 Hz, 3H) |
| II-7 | LCMS: calc. for C46H49N5O4: 735.4, found: [M + H]⁺ 736.3. |
| | ¹H NMR (400 MHz, CD₃OD) δ 7.65 (d, J = 8.4 Hz, 1H), 7.25 (d, J = 8.0 Hz, |
| | 2H), 7.17-7.08 (m, 3H), 6.89-6.80 (m, 4H), 6.70-6.62 (m, 4H), 6.52 (dd, J = |
| | 2.4, 8.3 Hz, 1H), 5.10 (dd, J = 5.2, 13.2 Hz, 1H), 4.48-4.32 (m, 3H), 3.98- |
| | 3.69 (m, 3H), 3.67-3.40 (m, 7H), 3.28-3.09 (m, 2H), 3.09-2.84 (m, 3H), |
| | 2.83-2.72 (m, 1H), 2.56-2.05 (m, 9H), 1.84 (d, J = 12.6 Hz, 3H). |
| II-8 | LCMS: calc. for C46H49N5O4: 735.4, found: [M + H]⁺ 736.3. |
| | ¹H NMR (400 MHz, CD₃OD-d₄) δ 7.66 (d, J = 8.2 Hz, 1H), 7.27 (d, J = 7.0 |
| | Hz, 2H), 7.20-7.05 (m, 3H), 6.91-6.77 (m, 4H), 6.71-6.61 (m, 4H), 6.53 |
| | (dd, J = 2.4, 8.3 Hz, 1H), 5.10 (dd, J = 5.2, 13.3 Hz, 1H), 4.50-4.30 (m, 3H), |
| | 3.93-3.70 (m, 3H), 3.66-3.41 (m, 7H), 3.30-3.09 (m, 2H), 3.09-2.83 (m, |
| | 3H), 2.83-2.72 (m, 1H), 2.61-1.99 (m, 9H), 1.85 (d, J = 13.0 Hz, 3H) |
| II-9 | LCMS: calc. for C₄₅H₄₉N₅O₄: 723.92, found: [M + H]⁺ 724.4. |
| | ¹H NMR (400 MHz, DMSO-d₆) δ 10.96 (s, 1H), 9.12 (br s, 1H), 8.14 (s, 1H), |
| | 7.50 (d, J = 8.2 Hz, 1H), 7.21-7.06 (m, 3H), 6.83 (br d, J = 6.6 Hz, 2H), 6.69- |
| | 6.59 (m, 2H), 6.56-6.44 (m, 5H), 6.19 (d, J = 8.6 Hz, 2H), 5.04 (dd, J = 5.1, |
| | 13.2 Hz, 1H), 4.37-4.26 (m, 1H), 4.22-4.09 (m, 2H), 4.02 (br t, J = 7.2 Hz, |
| | 2H), 3.62 (br s, 2H), 3.50 (br d, J = 9.5 Hz, 2H), 3.43-3.21 (m, 5H), 3.03-2.82 |
| | (m, 3H), 2.60 (br s, 1H), 2.41-2.28 (m, 1H), 2.20-2.08 (m, 5H), 2.01-1.89 |
| | (m, 1H), 1.72 (br d, J = 10.7 Hz, 3H), 1.58 (br s, 1H), 1.24-1.07 (m, 2H) |
| II-10 | LCMS: calc. for C₄₆H₅₁O₄N₅: 737.93, found: [M + H]⁺ 738.4. |
| | ¹H NMR (400 MHz, DMSO-d₆) δ ppm = 10.96 (s, 1H), 9.11 (s, 1H), 8.14 (s, |
| | 1H), 7.52 (br d, J = 8.3 Hz, 1H), 7.18-7.13 (m, 3H), 7.01 (br s, 2H), 6.84 (br d, |
| | J = 6.9 Hz, 2H), 6.65 (d, J = 8.2 Hz, 1H), 6.60 (s, 1H), 6.54 (br d, J = 8.3 Hz, 2H), |
| | 6.49 (br d, J = 8.2 Hz, 1H), 6.21 (br d, J = 8.2 Hz, 2H), 5.05 (br dd, J = 5.2, 13.2 |
| | Hz, 1H), 4.37-4.29 (m, 1H), 4.24-4.12 (m, 3H), 3.53 (br d, J = 11.3 Hz, 3H), |
| | 3.27 (br s, 1H), 3.01-2.86 (m, 5H), 2.76 (br s, 1H), 2.61 (br s, 3H), 2.40-2.30 |
| | (m, 2H), 2.24-2.06 (m, 4H), 2.01-1.92 (m, 1H), 1.82-1.68 (m, 4H), 1.18 (br |
| | d, J = 10.3 Hz, 2H), 1.09 (br d, J = 5.7 Hz, 3H) |
| II-12 | LCMS: calc. for C₄₆H₅₁N₅O₄: 737.95, found: [M + H]⁺ 738.4. |
| | ¹H NMR (400 MHz, DMSO-d₆) δ ppm = 10.96 (s, 1H), 9.12 (br s, 1H), 8.15 (s, |
| | 1H), 7.55-7.47 (m, 1H), 7.19-7.09 (m, 3H), 7.00 (br s, 2H), 6.84 (br d, J = 6.7 |
| | Hz, 2H), 6.68-6.59 (m, 2H), 6.57-6.47 (m, 3H), 6.20 (d, J = 8.6 Hz, 2H), 5.05 |
| | (dd, J = 5.1, 13.3 Hz, 1H), 4.37-4.28 (m, 1H), 4.24-4.10 (m, 3H), 3.54-3.45 |
| | (m, 5H), 3.09-2.83 (m, 6H), 2.77 (br d, J = 10.7 Hz, 1H), 2.41-2.32 (m, 1H), |
| | 2.24-2.06 (m, 5H), 2.00-1.92 (m, 1H), 1.83-1.61 (m, 4H), 1.23-1.13 (m, |
| | 2H), 1.09 (d, J = 6.4 Hz, 3H) |
| II-13 | LCMS: calc. for C₄₄H₄₇N₅O₄: 709.36, found: [M + H]⁺ 710.5. |
| | ¹HNMR (400 MHz, DMSO-d6): 10.95 (s, 1H), 9.08 (s, 1H), 7.52 (d, J = 8.8 |
| | Hz, 1H), 7.18-7.04 (m, 5H), 6.85 (br d, J = 7.6 Hz, 2H), 6.64-6.53 (m, 2H), |
| | 6.50-6.44 (m, 1H), 6.20-6.12 (m, 4H), 5.05 (dd, J = 5.0, 13.4 Hz, 1H), 4.31- |
| | 4.22 (m, 1H), 4.14-4.08 (m, 1H), 3.20-3.06 (m, 3H), 3.02-2.80 (m, 5H), |
| | 2.78-2.69 (m, 2H), 2.64 (br s, 3H), 2.41-2.35 (m, 4H), 2.31-2.24 (m, 1H), |
| | 2.17-1.94 (m, 4H), 1.75-1.62 (m, 2H), 1.34-1.12 (m, 3H). |
| II-14 | LCMS: calc. for C₄₅H₄₉N₅O₄: 723.92, found: [M + H]⁺ 724.5. |
| | ¹H NMR (400 MHz, DMSO-d₆) δ 11.49 (br s, 1H), 10.96 (s, 1H), 7.54 (d, |
| | J = 8.2 Hz, 1H), 7.44 (br s, 2H), 7.21-7.08 (m, 3H), 6.81 (br d, J = 6.7 Hz, 2H), |
| | 6.69-6.47 (m, 7H), 5.04 (dd, J = 4.9, 13.3 Hz, 1H), 4.41-4.15 (m, 6H), 3.54- |
| | 3.33 (m, 5H), 3.10-2.84 (m, 6H), 2.78 (br d, J = 3.1 Hz, 3H), 2.60 (br s, 1H), |
| | 2.40-2.16 (m, 4H), 2.13-1.82 (m, 5H), 1.73 (br d, J = 6.6 Hz, 1H) |
| II-95 | LCMS: calc. for C₄₈H₅₃N₅O₄: 763.4, found: [M + H]⁺ 764.6 |
| | ¹H NMR (400 MHz, DMSO-d₆) δ = 10.95 (s, 1H), 9.13 (s, 1H), 8.14 (s, |
| | 0.36H), 7.50 (d, J = 8.2 Hz, 1H), 7.22-7.07 (m, 3H), 6.84 (br d, J = 6.4 Hz, 2H), |
| | 6.67-6.58 (m, 2H), 6.57-6.43 (m, 5H), 6.21 (d, J = 8.7 Hz, 2H), 5.04 (dd, |
| | J = 5.1, 13.3 Hz, 1H), 4.36-4.27 (m, 1H), 4.22-4.11 (m, 2H), 3.85-3.61 (m, |
| | 4H), 3.53 (br d, J = 9.8 Hz, 2H), 3.28 (br s, 6H), 3.05-2.82 (m, 4H), 2.75-2.57 |
| | (m, 4H), 2.43-2.23 (m, 1H), 2.18-2.03 (m, 1H), 2.02-1.83 (m, 5H), 1.75 (br |
| | d, J = 11.6 Hz, 3H), 1.29-1.12 (m, 2H) |
| II-96 | LCMS: calc. for C₄₈H₅₃N₅O₄: 763.4, found: [M + H]⁺ 764.4 |
| | ¹H NMR (400 MHz, DMSO-d₆) δ = 10.94 (s, 1H), 9.12 (br s, 1H), 8.15 (s, |
| | 0.58H), 7.48 (d, J = 8.5 Hz, 1H), 7.21-7.09 (m, 3H), 6.84 (br d, J = 6.8 Hz, 2H), |
| | 6.69-6.58 (m, 2H), 6.56-6.43 (m, 5H), 6.20 (br d, J = 8.5 Hz, 2H), 5.03 (dd, |
| | J = 4.9, 13.2 Hz, 1H), 4.35-4.26 (m, 1H), 4.21-4.09 (m, 2H), 3.63 (s, 3H), |
| | 3.49 (br s, 4H), 3.29-3.24 (m, 5H), 3.04-2.83 (m, 3H), 2.71-2.55 (m, 2H), |
| | 2.36 (br d, J = 13.3 Hz, 2H), 2.18 (br s, 2H), 2.14-2.04 (m, 1H), 1.95 (br s, |
| | 1H), 1.81-1.68 (m, 6H), 1.61 (br s, 1H), 1.14 (br d, J = 11.8 Hz, 2H) |
| II-15 | LCMS: calc. for C₄₆H₅₁N₅O₄: 737.39, found: [M + H]⁺ 738.3. |
| | ¹H NMR (400 MHz, DMSO-d₆): δ = 10.97 (s, 1H), 9.13 (s, 1H), 7.63-7.53 |
| | (m, 1H), 7.21-7.09 (m, 5H), 6.84 (br d, J = 6.7 Hz, 2H), 6.66-6.60 (m, 2H), |
| | 6.56 (br d, J = 6.9 Hz, 2H), 6.49 (dd, J = 2.4, 8.3 Hz, 1H), 6.21 (br d, J = 8.2 Hz, |
| | 2H), 5.06 (br dd, J = 4.1, 12.9 Hz, 1H), 4.35 (br d, J = 16.9 Hz, 1H), 4.28-4.18 |
| | (m, 1H), 4.14 (br d, J = 4.5 Hz, 1H), 3.75-3.49 (m, 3H), 3.30 (br d, J = 14.1 Hz, |
| | 2H), 3.14 (br s, 1H), 3.06-2.85 (m, 5H), 2.60 (br s, 4H), 2.39 (br d, J = 12.9 |
| | Hz, 2H), 2.24 (br s, 1H), 2.18-2.05 (m, 1H), 1.96 (br d, J = 5.1 Hz, 4H), 1.70 |
| | (br s, 2H), 1.40 (br s, 2H), 1.36-1.23 (m, 2H), 1.05 (br s, 1H) |
| II-16 | LCMS: calc. for C₄₅H₄₈FN₅O₄: 741.3, found: [M + H]⁺ 742.4. |
| | ¹H NMR (400 MHz, DMSO-d₆) δ = 11.00 (s, 1H), 9.12 (s, 1H), 8.14 (s, 1H), |
| | 7.49 (d, J = 8.1 Hz, 1H), 7.21-7.08 (m, 4H), 6.84 (br d, J = 6.7 Hz, 2H), 6.68- |
| | 6.58 (m, 2H), 6.57-6.43 (m, 3H), 6.20 (d, J = 8.7 Hz, 2H), 5.08 (dd, J = 5.1, |
| | 13.2 Hz, 1H), 4.49 (d, J = 17.0 Hz, 1H), 4.32 (d, J = 16.9 Hz, 1H), 4.13 (br d, |
| | J = 4.8 Hz, 1H), 3.57-3.46 (m, 2H), 3.27 (br s, 2H), 3.15 (br s, 4H), 3.03-2.85 |
| | (m, 3H), 2.55 (br s, 6H), 2.24 (br s, 2H), 2.18-2.03 (m, 2H), 2.02-1.92 (m, |
| | 1H), 1.81-1.59 (m, 4H), 1.26-1.09 (m, 2H) |
| II-17 | LCMS: calc. for C₄₆H₅₁N₅O₄: 791.44, found: [M + H]⁺ 792.5. |
| | ¹H NMR (400 MHz, DMSO-d₆) δ = 10.94 (s, 1 H), 9.11 (br s, 1 H), 8.17 (s, |
| | 0.694 H), 7.49 (d, J = 8.94 Hz, 1 H), 6.99-7.19 (m, 5 H), 6.83 (br d, J = 6.68 |
| | Hz, 2 H), 6.57-6.70 (m, 2 H), 6.42-6.56 (m, 3 H), 6.19 (d, J = 8.58 Hz, 2 H), |
| | 5.04 (dd, J = 13.17, 5.07 Hz, 1 H), 4.01-4.39 (m, 3 H), 3.48 (br s, 4 H), 3.24- |
| | 3.28 (m, 4 H), 2.83-3.03 (m, 3 H), 2.58-2.70 (m, 1 H), 2.39 (br s, 4 H), 2.30- |
| | 2.35 (m, 1 H), 2.21 (br d, J = 6.56 Hz, 2 H), 2.09 (br dd, J = 11.27, 6.26 Hz, 1 |
| | H), 1.90-2.00 (m, 1 H), 1.71 (br d, J = 11.32 Hz, 3 H), 1.60 (br s, 1 H), 1.50 |
| | (br s, 8 H), 1.06-1.18 (m, 2 H) |
| II-18 | LCMS: calc. for C₄₆H₅₁N₅O₄: 737.39, found: [M + 56]⁺ 792.5. |
| | ¹H NMR (400 MHz, METHANOL-d₆) δ = 8.25 (br s, 0.108 H), 7.63 (d, |
| | J = 9.06 Hz, 1 H), 7.04-7.15 (m, 5 H), 6.77-6.85 (m, 2 H), 6.59-6.72 (m, 4 |
| | H), 6.51 (dd, J = 8.23, 2.38 Hz, 1 H), 6.32 (d, J = 8.58 Hz, 2 H), 5.10 (dd, |
| | J = 13.29, 5.19 Hz, 1 H), 4.30-4.50 (m, 2 H), 4.19 (d, J = 4.89 Hz, 1 H), 3.57 |
| | (br d, J = 10.49 Hz, 2 H), 3.37-3.43 (m, 4 H), 3.31-3.37 (m, 4 H), 3.17 (br s, |
| | 1 H), 3.08 (br d, J = 6.91 Hz, 2 H), 2.97-3.04 (m, 2 H), 2.84-2.95 (m, 1 H), |
| | 2.73-2.82 (m, 1 H), 2.64 (br t, J = 11.32 Hz, 2 H), 2.45 (qd, J = 13.21, 4.71 Hz, |
| | 1 H), 2.09-2.29 (m, 2 H), 1.98 (br d, J = 3.34 Hz, 2 H), 1.58-1.91 (m, 10 H), |
| | 1.35-1.51 (m, 2 H) |
| | SFC: retention time, 4.756; Area, 97.94% |
| II-19 | LCMS: calc. for C₄₅H₄₈FN₅O₄: 741.37, found: [M + H]⁺ 742.4. |
| | ¹H NMR (400 MHz, DMSO-d₆) δ = 10.99 (s, 1H), 9.13 (br s, 1H), 8.15 (s, |
| | 0.28H), 7.43 (d, J = 11.6 Hz, 1H), 7.23 (d, J = 7.5 Hz, 1H), 7.19-7.07 (m, 3H), |
| | 6.84 (br d, J = 6.6 Hz, 2H), 6.71-6.59 (m, 2H), 6.57-6.45 (m, 3H), 6.20 (d, |
| | J = 8.6 Hz, 2H), 5.08 (dd, J = 5.1, 13.2 Hz, 1H), 4.42-4.32 (m, 1H), 4.29-4.20 |
| | (m, 1H), 4.13 (br d, J = 4.9 Hz, 1H), 3.51 (br d, J = 9.7 Hz, 5H), 3.11 (br s, 4H), |
| | 3.04-2.84 (m, 3H), 2.73-2.56 (m, 4H), 2.45-2.30 (m, 2H), 2.21 (br d, J = 6.9 |
| | Hz, 2H), 2.17-2.04 (m, 1H), 2.03-1.93 (m, 1H), 1.84-1.56 (m, 4H), 1.26- |
| | 1.08 (m, 2H) |
| II-22 | LCMS: calc. for C46H49N5O4: 723.4, found: [M + H]⁺ 724.4. |
| | ¹H NMR (500 MHz, CD₃OD) δ 8.13 (s, 1H), 7.69 (d, J = 9.3 Hz, 1H), 7.18- |
| | 7.07 (m, 5H), 6.81 (d, J = 6.7 Hz, 2H), 6.71-6.63 (m, 4H), 6.51 (d, J = 8.4 Hz, |
| | 1H), 6.33 (d, J = 8.4 Hz, 2H), 5.11 (dd, J = 5.2, 13.3 Hz, 1H), 4.50-4.34 (m, |
| | 2H), 4.20 (d, J = 4.6 Hz, 1H), 3.70-3.46 (m, 4H), 3.38 (d, J = 11.7 Hz, 4H), |
| | 3.17 (br s, 3H), 3.04-2.85 (m, 5H), 2.82-2.74 (m, 1H), 2.72-2.66 (m, 1H), |
| | 2.54-2.42 (m, 2H), 2.25-2.12 (m, 3H), 1.92-1.71 (m, 4H), 1.32-1.22 (m, |
| | 1H) |
| II-23 | LCMS: calc. for C46H49N5O4: 723.4, found: [M + H]⁺ 724.4. |
| | ¹H NMR (500 MHz, CD₃OD) δ 8.15 (s, 1H), 7.70-7.66 (m, 1H), 7.15-7.06 |
| | (m, 5H), 6.81 (d, J = 6.7 Hz, 2H), 6.70-6.64 (m, 4H), 6.53-6.48 (m, 1H), 6.33 |
| | (d, J = 8.5 Hz, 2H), 5.11 (dd, J = 5.2, 13.3 Hz, 1H), 4.49-4.36 (m, 2H), 4.20 (d, |
| | J = 4.9 Hz, 1H), 3.57-3.49 (m, 4H), 3.44-3.32 (m, 4H), 3.22 (s, 3H), 3.08- |
| | 2.85 (m, 5H), 2.82-2.73 (m, 1H), 2.72-2.64 (m, 1H), 2.53-2.42 (m, 2H), |
| | 2.27-2.13 (m, 3H), 1.91-1.69 (m, 4H), 1.29-1.20 (m, 1H) |
| II-24 | LCMS: calc. for C₄₅H₄₈FN₅O₄: 741.37, found: [M + H]⁺ 742.1. |
| | ¹H NMR (400 MHz, DMSO-d₆) δ = 10.96 (s, 1H), 9.16 (s, 1H), 8.14 (s, 1H), |
| | 7.56 (br d, J = 8.2 Hz, 1H), 7.23-7.07 (m, 4H), 6.88 (t, J = 6.9 Hz, 1H), 6.68- |
| | 6.54 (m, 4H), 6.51-6.40 (m, 2H), 6.25 (br d, J = 8.5 Hz, 2H), 5.06 (dd, J = 5.0, |
| | 13.2 Hz, 1H), 4.41-4.13 (m, 3H), 3.53 (br d, J = 10.6 Hz, 2H), 3.06-2.83 (m, |
| | 5H), 2.65-2.52 (m, 9H), 2.38 (br dd, J = 4.5, 13.1 Hz, 3H), 2.23-2.04 (m, |
| | 2H), 2.02-1.91 (m, 1H), 1.81 (br d, J = 9.9 Hz, 3H), 1.69 (br d, J = 8.7 Hz, 1H), |
| | 1.23 (br s, 2H) |
| II-25 | LCMS: calc. for C₄₅H₄₈FN₅O₄: 741.37, found: [M + H]⁺ 742.1. |
| | ¹H NMR(400 MHz, DMSO-d₆) δ = 10.95 (s, 1H), 9.15 (br s, 1H), 8.16 (s, 1H), |
| | 7.52 (br d, J = 8.6 Hz, 1H), 7.23-7.11 (m, 2H), 7.10-7.01 (m, 2H), 6.88 (br t, |
| | J = 7.2 Hz, 1H), 6.67-6.41 (m, 6H), 6.24 (br d, J = 8.6 Hz, 2H), 5.05 (br dd, |
| | J = 4.8, 13.1 Hz, 1H), 4.38-4.28 (m, 1H), 4.25-4.15 (m, 2H), 3.52 (br d, J = 6.9 |
| | Hz, 2H), 3.28 (br s, 6H), 3.07-2.80 (m, 5H), 2.61 (br s, 4H), 2.39 (br s, 1H), |
| | 2.19 (br d, J = 6.4 Hz, 2H), 2.15 (br s, 1H), 2.02-1.91 (m, 1H), 1.79-1.60 (m, |
| | 4H), 1.15 (br d, J = 9.8 Hz, 2H) |
| II-26 | LCMS: calc. for C₄₅H₄₈FN₅O₄: 741.3, found: [M + H]⁺ 742.3. |
| | ¹H NMR (400 MHz, DMSO-d₆) δ = 10.95 (s, 1H), 9.12 (s, 1H), 8.13 (s, 1H), |
| | 7.52 (d, J = 8.5 Hz, 1H), 7.20 (br d, J = 6.4 Hz, 1H), 7.06 (s, 2H), 6.93 (br d, |
| | J = 2.4 Hz, 1H), 6.74 (d, J = 7.6 Hz, 1H), 6.64 (d, J = 8.5 Hz, 1H), 6.61-6.54 |
| | (m, 4H), 6.48 (dd, J = 2.4, 8.3 Hz, 1H), 6.23 (d, J = 8.6 Hz, 2H), 5.04 (dd, J = |
| | 4.9, 13.4 Hz, 1H), 4.32 (d, J = 17.0 Hz, 1H), 4.24-4.12 (m, 2H), 3.53 (br d, J = |
| | 11.7 Hz, 2H), 3.30-3.20 (m, 5H), 2.92 (br d, J = 19.0 Hz, 3H), 2.60 (br d, J = |
| | 2.5 Hz, 3H), 2.49-2.45 (m, 3H), 2.43-2.31 (m, 2H), 2.23 (br s, 2H), 2.13- |
| | 2.04 (m, 1H), 1.96 (br d, J = 5.4 Hz, 1H), 1.73 (br s, 4H), 1.20-1.06 (m, 2H) |
| II-27 | LCMS: calc. for C₄₅H₄₈FN₅O₄: 741.3, found: [M + H]⁺ 742.3. |
| | ¹H NMR (400 MHz, DMSO-d₆) δ = 10.95 (s, 1H), 9.13 (br s, 1H), 8.14 (s, |
| | 1H), 7.52 (d, J = 8.6 Hz, 1H), 7.23-7.16 (m, 1H), 7.11-7.02 (m, 2H), 6.99- |
| | 6.90 (m, 1H), 6.75 (br d, J = 7.5 Hz, 1H), 6.67-6.53 (m, 5H), 6.53-6.45 (m, |
| | 1H), 6.24 (br d, J = 8.5 Hz, 2H), 5.05 (dd, J = 5.1, 13.2 Hz, 1H), 4.37-4.28 |
| | (m, 1H), 4.25-4.11 (m, 2H), 3.53 (br d, J = 11.3 Hz, 3H), 3.29 (br s, 9H), 2.99- |
| | 2.85 (m, 3H), 2.65-2.60 (m, 1H), 2.59-2.55 (m, 3H), 2.45-2.29 (m, 3H), |
| | 2.20 (br d, J = 6.8 Hz, 2H), 2.08 (br s, 1H), 2.03-1.90 (m, 1H), 1.81-1.62 |
| | (m, 4H), 1.23-1.10 (m, 2H) |
| II-101 | LCMS: calc. for C₄₅H₄₈FN₅O₄: 741.37, found: [M + H]⁺742.4. |
| | ¹H NMR (400 MHz, DMSO-d₆) δ = 11.00-10.92 (m, 1H), 9.56-8.77 (m, |
| | 1H), 8.18-8.12 (m, 0.822H), 7.56-7.50 (m, 1H), 7.08-7.03 (m, 2H), 7.00- |
| | 6.94(m, 2H), 6.88-6.82 (m, 2H), 6.65 (s, 4H), 6.51-6.46 (m, 1H), 6.24-6.19 |
| | (m, 2H), 5.09-5.01 (m, 1H), 4.36-4.30 (m, 1H), 4.24-4.17 (m, 1H), 4.13- |
| | 4.09(m, 1H), 3.53 (br d, J = 8.8 Hz, 4H), 3.05-2.83 (m, 4H), 2.72-2.52 (m, |
| | 6H), 2.44-2.27 (m, 2H), 2.21 (br d, J = 6.9 Hz, 5H), 1.79-1.61 (m, 4H), 1.34- |
| | 1.04 (m, 3H) |
| II-94 | LCMS: calc. for C₄₅H₄₈FN₅O₄: 741.37, found: [M + H]⁺742.4. |
| | ¹H NMR (400 MHz, DMSO-d₆) δ = 10.97-10.94 (m, 1H), 9.15-9.09 (m, |
| | 1H), 8.15-8.14 (m, 0.396H), 7.55-7.51 (m, 1H), 7.08-7.04 (m, 2H), 7.00- |
| | 6.95(m, 2H), 6.87-6.83 (m, 2H), 6.64 (br d, J = 8.5 Hz, 1H), 6.61-6.56 (m, |
| | 3H), 6.50-6.47 (m, 1H), 6.24-6.20 (m, 2H), 5.08-5.02 (m, 1H), 4.36-4.30 |
| | (m, 1H), 4.24-4.18 (m, 1H), 4.13-4.09 (m, 1H), 3.58-3.49 (m, 3H), 3.04- |
| | 2.82 (m, 4H), 2.61 (br s, 5H), 2.45-2.19 (m, 5H), 2.17-1.87 (m, 3H), 1.83- |
| | 1.60(m, 5H), 1.24 (br s, 3H) |
| II-28 | LCMS: calc. for C₄₆H₅₁N₅O₄: 737.39, found: [M + H]⁺ 738.4. |
| | ¹H NMR (400 MHz, DMSO-d₆) δ = 10.95 (s, 1H), 9.11 (s, 1H), 8.14 (s, |
| | 0.42H), 7.59-7.44 (m, 1H), 7.15-6.99 (m, 3H), 6.93 (br d, J = 7.6 Hz, 1H), |
| | 6.68-6.53 (m, 6H), 6.49 (dd, J = 2.2, 8.3 Hz, 1H), 6.21 (d, J = 8.5 Hz, 2H), 5.05 |
| | (dd, J = 5.0, 13.2 Hz, 1H), 4.40-4.30 (m, 1H), 4.26-4.16 (m, 1H), 4.10 (br d, |
| | J = 4.8 Hz, 1H), 3.53 (br d, J = 11.9 Hz, 2H), 3.23 (br d, J = 13.4 Hz, 8H), 3.02- |
| | 2.84 (m, 3H), 2.76-2.60 (m, 3H), 2.44-2.27 (m, 3H), 2.26-2.02 (m, 5H), |
| | 2.01-1.91 (m, 1H), 1.85-1.62 (m, 4H), 1.19 (br d, J = 11.8 Hz, 2H) |
| II-29 | LCMS: calc. for C₄₆H₅₁N₅O₄: 737.39, found: [M + H]⁺ 738.4. |
| | ¹H NMR (400 MHz, DMSO-d₆) δ = 10.96 (s, 1H), 9.11 (s, 1H), 8.14 (s, 1H), |
| | 7.53 (br d, J = 8.6 Hz, 1H), 7.13-6.98 (m, 3H), 6.93 (br d, J = 7.3 Hz, 1H), 6.65 |
| | (d, J = 8.3 Hz, 1H), 6.63-6.53 (m, 5H), 6.48 (dd, J = 2.3, 8.2 Hz, 1H), 6.21 (d, |
| | J = 8.7 Hz, 2H), 5.05 (dd, J = 5.1, 13.2 Hz, 1H), 4.38-4.28 (m, 1H), 4.25-4.17 |
| | (m, 1H), 4.10 (br d, J = 5.0 Hz, 1H), 3.53 (br d, J = 11.8 Hz, 2H), 3.31-3.19 (m, |
| | 8H), 3.02-2.85 (m, 3H), 2.69-2.59 (m, 2H), 2.41-2.31 (m, 3H), 2.27-2.01 |
| | (m, 6H), 2.01-1.91 (m, 1H), 1.83-1.59 (m, 4H), 1.27-1.11 (m, 2H) |
| II-32 | LCMS: calc. for C₄₄H₄₇N₅O₄: 709.36, found: [M + H]⁺ 710.5. |
| | 1H NMR (500 MHz, DMSO-d6) δ = 10.96 (s, 1H), 9.10 (s, 1H), 8.17 (s, 1H), |
| | 7.52 (d, J = 8.4 Hz, 1H), 7.20-7.10 (m, 3H), 7.09-7.03 (m, 2H), 6.86 (d, J = |
| | 7.0 Hz, 2H), 6.65-6.58 (m, 2H), 6.47 (dd, J = 2.5, 8.3 Hz, 1H), 6.21-6.12 |
| | (m, 4H), 5.05 (dd, J = 5.1, 13.4 Hz, 1H), 4.33 (d, J = 16.8 Hz, 1H), 4.23-4.18 |
| | (m, 1H), 4.11 (d, J = 5.0 Hz, 1H), 3.09 (s, 12H), 3.01-2.83 (m, 4H), 2.36 (br |
| | d, J = 10.2 Hz, 5H), 2.12 (dt, J = 5.7, 12.2 Hz, 1H), 2.07-2.01 (m, 1H), 1.98- |
| | 1.94 (m, 1H), 1.74-1.63 (m, 2H) |
| II-33 | LCMS: calc. for C₄₄H₄₇N₅O₄: 709.36, found: [M + H]⁺ 710.5. |
| | 1H NMR (500 MHz, DMSO-d6) δ = 10.96 (s, 1H), 9.10 (br s, 1H), 8.21 (s, |
| | 1H), 7.52 (d, J = 8.4 Hz, 1H), 7.19-7.10 (m, 3H), 7.10-7.04 (m, 2H), 6.85 |
| | (br d, J = 7.9 Hz, 2H), 6.65-6.56 (m, 2H), 6.47 (dd, J = 2.4, 8.4 Hz, 1H), 6.24- |
| | 6.09 (m, 4H), 5.05 (dd, J = 5.0, 13.3 Hz, 1H), 4.37-4.29 (m, 1H), 4.28-4.16 |
| | (m, 1H), 4.11 (d, J = 4.7 Hz, 1H), 3.32-3.03 (m, 12H), 3.01-2.83 (m, 4H), |
| | 2.75-2.57 (m, 3H), 2.40-2.32 (m, 4H), 2.18-1.96 (m, 3H), 1.74-1.62 (m, |
| | 2H). |
| II-34 | LCMS: calc. for C₄₆H₅₁N₅O: 737.3, found: [M + H]⁺ 738.4 |
| | ¹H NMR (400 MHz, DMSO-d₆) δ = 10.94 (s, 1H), 9.11 (s, 1H), 7.52 (d, J = |
| | 8.8 Hz, 1H), 7.16 (d, J = 7.5 Hz, 1H), 7.10-6.99 (m, 3H), 6.80 (t, J = 7.2 Hz, |
| | 1H), 6.66 (d, J = 8.3 Hz, 1H), 6.60 (s, 1H), 6.56-6.47 (m, 3H), 6.16 (br d, J = |
| | 8.3 Hz, 3H), 5.05 (dd, J = 5.1, 13.4 Hz, 1H), 4.36-4.29 (m, 1H), 4.23-4.14 |
| | (m, 2H), 3.51 (br d, J = 12.0 Hz, 2H), 3.44-3.38 (m, 2H), 3.30-3.28 (m, 5H), |
| | 2.96 (br s, 2H), 2.90 (br s, 1H), 2.68 (s, 1H), 2.45-2.41 (m, 4H), 2.41-2.29 |
| | (m, 2H), 2.22 (br s, 3H), 1.96 (br d, J = 5.5 Hz, 1H), 1.76 (br d, J = 11.8 Hz, |
| | 2H), 1.62 (br s, 2H), 1.16 (br d, J = 8.5 Hz, 2H) |
| II-35 | LCMS: calc. for C₄₆H₅₁N₅O₄: 737.4, found: [M + H]⁺ 738.2 |
| | ¹H NMR (400 MHz, DMSO-d₆) δ = 10.95 (s, 1H), 9.13 (br s, 1H), 8.14 (s, |
| | 1H), 7.52 (d, J = 8.6 Hz, 1H), 7.23-7.16 (m, 1H), 7.11-7.02 (m, 2H), 6.99- |
| | 6.90 (m, 1H), 6.75 (br d, J = 7.5 Hz, 1H), 6.67-6.53 (m, 5H), 6.53-6.45 (m, |
| | 1H), 6.24 (br d, J = 8.5 Hz, 2H), 5.05 (dd, J = 5.1, 13.2 Hz, 1H), 4.37-4.28 |
| | (m, 1H), 4.25-4.11 (m, 2H), 3.53 (br d, J = 11.3 Hz, 3H), 3.29 (br s, 9H), 2.99- |
| | 2.85 (m, 3H), 2.65-2.60 (m, 1H), 2.59-2.55 (m, 3H), 2.45-2.29 (m, 3H), |
| | 2.20 (br d, J = 6.8 Hz, 2H), 2.08 (br s, 1H), 2.03-1.90 (m, 1H), 1.81-1.62 |
| | (m, 4H), 1.23-1.10 (m, 2H) |
| II-36 | LCMS: calc. for C₄₆H₅₁N₅O₄: 737.39, found: [M + H]⁺ 738.3. |
| | ¹H NMR: 1 H NMR (400 MHz, DMSO-d₆) δ = 10.96 (s, 1H), 9.26-8.98 (m, |
| | 1H), 8.17 (s, 0.313H), 7.54-7.46 (m, 1H), 7.13-7.03 (m, 2H), 6.96 (d, J = |
| | 7.9 Hz, 2H), 6.71 (d, J = 8.0 Hz, 2H), 6.64 (d, J = 8.3 Hz, 1H), 6.59 (d, J = 2.3 |
| | Hz, 1H), 6.55 (br d, J = 8.8 Hz, 2H), 6.47 (dd, J = 2.4, 8.3 Hz, 1H), 6.23 (d, |
| | J = 8.6 Hz, 2H), 5.05 (dd, J = 5.1, 13.2 Hz, 1H), 4.38-4.29 (m, 1H), 4.24-4.16 |
| | (m, 1H), 4.10 (br d, J = 4.8 Hz, 1H), 3.30-3.19 (m, 9H), 3.01-2.84 (m, 4H), |
| | 2.69-2.57 (m, 2H), 2.55 (s, 1H), 2.41-2.29 (m, 2H), 2.24-2.18 (m, 5H), 2.11- |
| | 2.03 (m, 1H), 2.00-1.92 (m, 1H), 1.76 (br d, J = 11.7 Hz, 2H), 1.70-1.61 |
| | (m, 2H), 1.24-1.13 (m, 2H). |
| II-37 | LCMS: calc. for C₄₆H₅₁N₅O₄: 737.93 found: [M + H]⁺ 738.3. |
| | ¹H NMR: ¹H NMR (400 MHz, DMSO-d₆) δ = 10.95 (s, 1H), 9.12 (br s, 1H), |
| | 8.15 (s, 0.336H), 7.52 (d, J = 8.8 Hz, 1H), 7.10-7.03 (m, 2H), 6.95 (d, J = 8.0 |
| | Hz, 2H), 6.71 (d, J = 8.0 Hz, 2H), 6.64 (d, J = 8.3 Hz, 1H), 6.61-6.53 (m, 3H), |
| | 6.47 (dd, J = 2.3, 8.3 Hz, 1H), 6.23 (d, J = 8.5 Hz, 2H), 5.04 (dd, J = 5.0, 13.1 |
| | Hz, 1H), 4.38-4.28 (m, 1H), 4.25-4.16 (m, 1H), 4.10 (d, J = 4.5 Hz, 1H), |
| | 3.30-3.20 (m, 9H), 3.01-2.83 (m, 4H), 2.70-2.53 (m, 3H), 2.41-2.28 (m, |
| | 2H), 2.24-2.19 (m, 5H), 2.08 (br dd, J = 7.3, 11.5 Hz, 1H), 2.00-1.93 (m, |
| | 1H), 1.76 (br d, J = 12.5 Hz, 2H), 1.70-1.61 (m, 2H), 1.21-1.11 (m, 2H) |
| II-38 | LCMS: calc. for C₄₅H₄₈FN₅O₄: 741.37, found: [M + H]⁺ 742.3. |
| | ¹H NMR (400 MHz, DMSO-d₆) δ = 10.99 (s, 1H), 9.11 (s, 1H), 8.14 (s, |
| | 0.24H), 7.43 (d, J = 11.7 Hz, 1H), 7.23 (d, J = 7.6 Hz, 1H), 7.18-7.09 (m, 3H), |
| | 6.84(br d, J = 6.6 Hz, 2H), 6.67-6.59 (m, 2H), 6.54 (d, J = 8.7 Hz, 2H), 6.48 |
| | (dd, J = 2.4, 8.2 Hz, 1H), 6.20 (d, J = 8.6 Hz, 2H), 5.08 (dd, J = 5.0, 13.4 Hz, 1H), |
| | 4.40-4.32 (m, 1H), 4.28-4.19 (m, 1H), 4.13 (d, J = 4.8 Hz, 1H), 3.52 (br d, |
| | J = 9.7 Hz, 2H), 3.28 (br s, 2H), 3.12 (br s, 4H), 3.04-2.85 (m, 3H), 2.61 (br s, |
| | 3H), 2.48-2.34 (m, 3H), 2.30-1.88 (m, 5H), 1.82-1.61 (m, 4H), 1.25-1.06 (m, 2H) |
| II-39 | LCMS: calc. for C₄₆H₄₉N₅O₄: 735.4, found: [M + H]⁺ 736.3. |
| | ¹H NMR (400 MHz, DMSO-d₆) δ = 10.94 (s, 1H), 9.11 (br s, 1H), 8.15 (s, |
| | 0.819H), 7.48 (d, J = 8.3 Hz, 1H), 7.17-7.09 (m, 3H), 6.82 (br d, J = 6.4 Hz, |
| | 2H), 6.66-6.58 (m, 2H), 6.53-6.44 (m, 5H), 6.19 (d, J = 8.6 Hz, 2H), 5.02 |
| | (dd, J = 5.0, 13.4 Hz, 1H), 4.32-4.26 (m, 1H), 4.20-4.10 (m, 2H), 3.96 (s, |
| | 4H), 3.47 (br d, J = 12.0 Hz, 1H), 2.98-2.84 (m, 4H), 2.61-2.57 (m, 5H), 2.42 |
| | (br s, 4H), 2.29 (br d, J = 6.6 Hz, 2H), 2.10 (td, J = 6.2, 12.2 Hz, 1H), 1.98-1.90 |
| | (m, 1H), 1.68 (br d, J = 9.1 Hz, 3H), 1.33 (br s, 1H), 1.20-1.07 (m, 2H) |
| II-103 | LC-MS (ESI⁺) m/z: 760.3 (M + H)⁺. |
| | ¹H NMR (400 MHz, METHANOL-d₄) δ 7.70 (d, J = 8.94 Hz, 1H), 7.15-7.20 |
| | (m, 2H), 7.04-7.12 (m, 3H), 6.93-7.00 (m, 2H), 6.69 (d, J = 8.34 Hz, 1H), 6.60 |
| | (d, J = 2.50 Hz, 1H), 6.39-6.52 (m, 3H), 5.11 (dd, J = 5.19, 13.29 Hz, 1H), 4.62 |
| | (br d, J = 6.08 Hz, 1H), 4.36-4.50 (m, 2H), 4.04 (br d, J = 10.85 Hz, 2H), 3.62- |
| | 3.79 (m, 4H), 3.32-3.43 (m, 3H), 3.24-3.30 (m, 2H), 3.19 (br d, J = 6.79 Hz, |
| | 2H), 2.84-3.05 (m, 5H), 2.73-2.83 (m, 1H), 2.33-2.55 (m, 2H), 2.11-2.24 (m, |
| | 2H), 1.97 (br d, J = 11.21 Hz, 2H), 1.85 (br d, J = 12.99 Hz, 1H), 1.52 (q, |
| | J = 12.32 Hz, 2H) |
| II-102 | LC-MS (ESI⁺) m/z: 760.3 (M + H)⁺. |
| | ¹H NMR (400 MHz, METHANOL-d₄) δ 7.70 (d, J = 8.94 Hz, 1H), 7.15-7.21 |
| | (m, 2H), 7.04-7.13 (m, 3H), 6.95-6.99 (m, 2H), 6.68 (d, J = 8.34 Hz, 1H), 6.55- |
| | 6.65 (m, 3H), 6.49 (dd, J = 2.62, 8.34 Hz, 1H), 5.11 (dd, J = 5.13, 13.35 Hz, 1H), |
| | 4.66 (d, J = 6.20 Hz, 1H), 4.37-4.50 (m, 2H), 4.05 (br d, J = 10.85 Hz, 2H), 3.63- |
| | 3.80 (m, 4H), 3.33-3.47 (m, 3H), 3.24-3.30 (m, 2H), 3.21 (d, J = 6.79 Hz, 2H), |
| | 3.10 (br t, J = 12.58 Hz, 2H), 3.02 (br dd, J = 3.40, 8.52 Hz, 2H), 2.84-2.96 (m, |
| | 1H), 2.73-2.82 (m, 1H), 2.33-2.54 (m, 2H), 2.24 (br s, 1H), 2.16 (tdd, J = 2.61, |
| | 5.25, 12.67 Hz, 1H), 1.99-2.07 (m, 2H), 1.87 (br d, J = 12.04 Hz, 1H), 1.61 (q, |
| | J = 12.16 Hz, 2H). |
| II-47 | LC-MS (ESI+) m/z: 750.3 (M + H)⁺ |
| | ¹H NMR (400 MHz, DMSO-d₆) δ = 10.96 (s, 1H), 9.12 (s, 2H), 8.13 (s, |
| | 0.039H), 7.56 (br s, 1H), 7.47 (d, J = 8.0 Hz, 1H), 7.15-7.09 (m, 5H), 6.82 (br |
| | d, J = 6.8 Hz, 2H), 6.69 (d, J = 8.3 Hz, 1H), 6.60 (s, 1H), 6.50 (br d, J = 7.8 Hz, |
| | 1H), 6.40-6.38 (m, J = 8.3 Hz, 1H), 6.21-6.15 (m, J = 8.3 Hz, 2H), 5.06 (br dd, |
| | J = 5.0, 13.1 Hz, 1H), 4.34 (br d, J = 17.1 Hz, 1H), 4.21 (br d, J = 17.1 Hz, 1H), |
| | 4.13-4.09 (m, 2H), 3.98-3.90 (m, 1H), 3.57 (br s, 1H), 3.29-3.23 (m, |
| | 1H), 3.13 (br d, J = 13.3 Hz, 2H), 3.04-2.79 (m, 5H), 2.62-2.52 (m, 1H), |
| | 2.38 (br d, J = 13.3 Hz, 4H), 2.28 (s, 3H), 2.15-2.05 (m, 1H), 2.00-1.88 (m, |
| | 3H), 1.80 (br s, 2H), 1.70 (br s, 1H), 1.42-1.30 (m, 3H) |
| II-56 | LC-MS (ESI⁺) m/z: 750.4 (M + H)⁺. |
| | ¹H NMR (400 MHz, DMSO-d₆) δ = 10.96 (s, 1H), 9.12 (s, 1H), 8.13 (s, |
| | 0.036H), 7.57 (br d, J = 8.3 Hz, 1H), 7.47 (d, J = 7.8 Hz, 1H), 7.15-7.09 (m, |
| | 5H), 6.82 (br d, J = 7.0 Hz, 2H), 6.69 (d, J = 8.5 Hz, 1H), 6.61 (s, 1H), 6.50 (br |
| | d, J = 8.3 Hz, 1H), 6.39 (br d, J = 8.5 Hz, 1H), 6.18 (br d, J = 8.3 Hz, 2H), 5.06 (br |
| | dd, J = 4.6, 13.2 Hz, 1H), 4.34 (br d, J = 17.1 Hz, 1H), 4.21 (br d, J = 17.1 Hz, |
| | 1H), 4.11 (br s, 3H), 3.93 (br s, 1H), 3.57 (br s, 1H), 3.26 (br d, J = 13.3 |
| | Hz, 1H), 3.16 (br s, 2H), 3.04-2.77 (m, 5H), 2.63-2.55 (m, 1H), 2.39 (br dd, |
| | J = 8.5, 12.8 Hz, 4H), 2.28 (s, 2H), 2.16-2.05 (m, 1H), 2.01-1.87 (m, 3H), |
| | 1.80 (br s, 2H), 1.69 (br s, 1H), 1.46-1.23 (m, 4H) |
| II-57 | LCMS: calc. for C₄₆H₅₁N₅O₄: 737.9, found: [M + H]⁺ 738.5. |
| | ¹HNMR (400 MHz, MeOD-d4): δ 8.29 (s, 1H), 7.58 (d, J = 8.5 Hz, 1H), 7.16- |
| | 7.04 (m, 5H), 6.81 (dd, J = 2.1, 7.2 Hz, 2H), 6.71-6.60 (m, 4H), 6.52 (dd, J = |
| | 2.6, 8.3 Hz, 1H), 6.32 (d, J = 8.7 Hz, 2H), 4.61 (d, J = 2.7 Hz, 2H), 4.19 (d, J = |
| | 5.0 Hz, 1H), 3.59-3.44 (m, 6H), 3.34 (d, J = 1.9 Hz, 1H), 3.07-2.91 (m, |
| | 6H), 2.87-2.73 (m, 2H), 2.71-2.59 (m, 5H), 2.30-2.15 (m, 1H), 2.03-1.95 |
| | (m, 1H), 1.88 (d, J = 11.9 Hz, 3H), 1.76 (s, 4H), 1.38 (q, J = 10.8 Hz, 2H). |
| II-58 | LCMS: calc. for C₄₆H₅₁N₅O₄: 737.9, found: [M + H]⁺ 738.5. |
| | ¹HNMR (400 MHz, MeOD-d4): δ 8.26 (s, 1H), 7.59 (d, J = 8.5 Hz, 1H), 7.16- |
| | 7.06 (m, 5H), 6.81 (dd, J = 2.0, 7.3 Hz, 2H), 6.73-6.61 (m, 4H), 6.52 (dd, J = |
| | 2.6, 8.4 Hz, 1H), 6.32 (d, J = 8.7 Hz, 2H), 4.62 (d, J = 3.1 Hz, 2H), 4.19 (d, |
| | J = 5.0 Hz, 1H), 3.60-3.48 (m, 6H), 3.34 (d, J = 1.7 Hz, 1H), 3.16-2.92 (m, |
| | 6H), 2.88-2.73 (m, 4H), 2.69-2.58 (m, 3H), 2.29-2.16 (m, 1H), 2.02-1.95 |
| | (m, 1H), 1.88 (d, J = 10.4 Hz, 3H), 1.76 (s, 4H), 1.46-1.34 (m, 2H). |
| II-99 | LCMS: calc. for C₄₅H₄₉N₅O₄: 723.9, found: [M + H]⁺ 724.5. |
| | ¹H NMR (400 MHz, MeOD-d4): δ 8.28 (s, 1H), 7.66 (d, J = 9.2 Hz, 1H), 7.19- |
| | 7.05 (m, 5H), 6.81 (dd, J = 2.0, 7.3 Hz, 2H), 6.73-6.60 (m, 4H), 6.55-6.48 |
| | (m, 1H), 6.52 (dd, J = 2.6, 8.3 Hz, 1H), 6.36-6.29 (m, 1H), 6.32 (d, J = 8.7 |
| | Hz, 2H), 5.10 (dd, J = 5.1, 13.4 Hz, 1H), 4.48-4.34 (m, 2H), 4.19 (d, J = 4.9 |
| | Hz, 1H), 3.61-3.42 (m, 6H), 3.34 (s, 1H), 3.08-2.84 (m, 7H), 2.81-2.73 (m, |
| | 1H), 2.68-2.55 (m, 4H), 2.46 (d, J = 4.8, 13.3 Hz, 1H), 2.30-2.09 (m, 2H), |
| | 1.94-1.71 (m, 4H), 1.48-1.30 (m, 2H). |
| II-59 | LCMS: calc. for C₄₆H₅₁N₅O₄₄: 737.39, found: [M + H]⁺ 738.5. |
| | ¹H NMR (400 MHz, DMSO-d₆) δ = 10.96 (s, 1H), 7.53 (br s, 1H), 7.21-7.01 |
| | (m, 5H), 6.85 (br d, J = 6.8 Hz, 2H), 6.77 (br d, J = 9.0 Hz, 2H), 6.66 (br d, J = 8.5 |
| | Hz, 1H), 6.54 (br s, 2H), 6.21 (br d, J = 7.5 Hz, 2H), 5.06 (br d, J = 9.0 Hz, 1H), |
| | 4.34 (br d, J = 16.1 Hz, 1H), 4.26-4.16 (m, 2H), 3.99 (br d, J = 12.5 Hz, 1H), |
| | 3.74 (s, 3H), 3.54 (br s, 3H), 3.29 (br s, 4H), 3.19-2.84 (m, 4H), 2.72-2.54 |
| | (m, 2H), 2.45-2.27 (m, 4H), 2.26-2.05 (m, 2H), 1.96 (br s, 2H), 1.87-1.56 |
| | (m, 4H), 1.34-1.07 (m, 2H) |
| II-60 | LC-MS (ESI⁺) m/z: 742.5(M + H)⁺ |
| | ¹H NMR (400 MHz, METHANOL-d₆) δ = 8.28 (s, 0.507H), 7.66 (d, J = 9.2 Hz, |
| | 1H), 7.25 (s, 1H), 7.14-7.09 (m, 5H), 7.07-7.01 (m, 1H), 6.89-6.80 (m, 3H), |
| | 6.65 (d, J = 8.7 Hz, 2H), 6.31 (d, J = 8.6 Hz, 2H), 5.10 (br dd, J = 5.1, 13.2 Hz, |
| | 1H), 4.59 (br s, 2H), 4.49-4.33 (m, 2H), 4.27 (d, J = 5.5 Hz, 1H), 3.56 (br |
| | d, J = 12.9 Hz, 2H), 3.46 (br s, 4H), 3.36 (br d, J = 6.0 Hz, 1H), 3.17-3.04 (m, |
| | 2H), 2.95-2.84 (m, 5H), 2.81-2.73 (m, 1H), 2.67-2.54 (m, 4H), 2.46 (dq, |
| | J = 4.8, 13.2 Hz, 1H), 2.34-2.22 (m, 1H), 2.21-2.10 (m, 1H), 1.86 (br t, |
| | J = 12.9 Hz, 4H), 1.43-1.29 (m, 2H) |
| II-51 | LC-MS (ESI+) m/z: 723.5(M + H)⁺. |
| | 1H NMR (400 MHz, METHANOL-d4) δ = 8.53-8.47 (m, 1H), 7.62 (d, J = |
| | 8.8 Hz, 1H), 7.13-7.05 (m, 5H), 6.86 (d, J = 8.1 Hz, 2H), 6.81-6.76 (m, 2H), |
| | 6.70-6.64 (m, 2H), 6.51 (dd, J = 2.5, 8.3 Hz, 1H), 6.38 (d, J = 8.1 Hz, 2H), |
| | 5.09 (br d, J = 8.2 Hz, 2H), 4.43-4.35 (m, 2H), 4.24 (br d, J = 4.9 Hz, 1H), |
| | 3.95 (br d, J = 13.1 Hz, 2H), 3.48-3.42 (m, 2H), 3.05-2.99 (m, 2H), 2.96- |
| | 2.63 (m, 10H), 2.47-2.38 (m, 1H), 2.21-2.09 (m, 2H), 2.04-1.74 (m, 9H), |
| | 1.45-1.34 (m, 2H). |
| II-61 | LC-MS (ESI+) m/z: 723.5(M + H)⁺. |
| | 1H NMR (400 MHz, METHANOL-d4) δ = 8.51 (s, 1H), 7.62 (d, J = 8.7 Hz, |
| | 1H), 7.12-7.04 (m, 5H), 6.86 (d, J = 8.1 Hz, 2H), 6.81-6.76 (m, 2H), 6.69- |
| | 6.64 (m, 2H), 6.51 (dd, J = 2.5, 8.2 Hz, 1H), 6.38 (d, J = 8.1 Hz, 2H), 5.09 (br |
| | dd, J = 5.2, 13.2 Hz, 2H), 4.42-4.33 (m, 2H), 4.24 (d, J = 5.0 Hz, 1H), 3.95 |
| | (br d, J = 12.8 Hz, 2H), 3.44 (br d, J = 11.2 Hz, 2H), 3.06-2.98 (m, 2H), 2.96- |
| | 2.59 (m, 10H), 2.49-2.40 (m, 1H), 2.22-2.09 (m, 2H), 2.08-1.72 (m, 9H), |
| | 1.46-1.34 (m, 2H). |
| II-62 | LCMS: calc. for C₄₅H₄₈BrN₅O₃, found: [M + H]⁺ 788.3. |
| | ¹H NMR (400 MHz, DMSO-d₆) δ = 10.93 (s, 1H), 8.16 (s, 1H), 7.52 (d, J = 8.6 |
| | Hz, 1H), 7.45 (s, 1H), 7.26-7.21 (m, 1H), 7.19-7.12 (m, 3H), 7.08-7.03 (m, |
| | 2H), 6.88-6.82 (m, 3H), 6.56 (br d, J = 8.6 Hz, 2H), 6.21 (d, J = 8.5 Hz, 2H), |
| | 5.05 (dd, J = 5.1, 13.3 Hz, 1H), 4.36-4.29 (m, 1H), 4.26-4.17 (m, 2H), 3.53 |
| | (br d, J = 9.2 Hz, 2H), 3.32 (br s, 4H), 3.10-2.82 (m, 3H), 2.61 (br s, 6H), 2.46- |
| | 2.27 (m, 2H), 2.22-2.10 (m, 3H), 2.00-1.89 (m, 1H), 1.76 (br d, J = 12.0 Hz, |
| | 3H), 1.64 (br s, 1H), 1.23-1.11 (m, 2H) |
| II-63 | LCMS: calc. for C₄₄H₄₆N₄O₆: 726.34, found: [M + H]⁺ 727.3. |
| | ¹HNMR (400 MHz, DMSO-d₆) δ = 10.95 (s, 1H), 9.12 (s, 1H), 7.55 (d, J = 8.3 |
| | Hz, 1H), 7.18-7.05 (m, 5H), 6.83 (br d, J = 6.9 Hz, 2H), 6.67-6.60 (m, 2H), |
| | 6.56-6.47 (m, 3H), 6.26 (d, J = 8.5 Hz, 2H), 5.06 (dd, J = 4.9, 13.4 Hz, 1H), |
| | 4.38-4.30 (m, 1H), 4.26-4.15 (m, 2H), 3.83 (br t, J = 6.0 Hz, 2H), 3.60 (br s, |
| | 4H), 3.27 (br d, J = 4.8 Hz, 2H), 3.03-2.84 (m, 3H), 2.61 (br s, 4H), 2.43-2.35 |
| | (m, 3H), 2.14-2.03 (m, 1H), 2.01-1.93 (m, 1H), 1.74-1.58 (m, 5H) |
| II-48 | LCMS: calc. for C46H51N5O4: 737.4, found: [M + H]⁺ 738.3. |
| | ¹H NMR (400 MHz, CD₃OD) δ 7.71-7.68 (m, 1H), 7.19-7.07 (m, 6H), 6.88- |
| | 6.78 (m, 3H), 6.73-6.64 (m, 3H), 6.56-6.46 (m, 2H), 5.11 (dd, J = 5.2, 13.3 |
| | Hz, 1H), 4.61-4.22 (m, 4H), 3.99 (s, 1H), 3.59-3.35 (m, 7H), 3.27-3.01 (m, |
| | 5H), 3.01-2.71 (m, 3H), 2.51-2.40 (m, 2H), 2.31-2.02 (m, 4H), 1.94-1.53 |
| | (m, 4H), 1.04-0.98 (m, 3H) |
| II-49 | LCMS: calc. for C46H51N5O4: 737.4, found: [M + H]⁺ 738.3. |
| | 1H NMR (500 MHz, MD3OD) δ 7.74-7.66 (m, 1H), 7.20-7.08 (m, 6H), 6.85- |
| | 6.79 (m, 2H), 6.72 (td, J = 4.2, 8.5 Hz, 2H), 6.75-6.59 (m, 1H), 6.58-6.52 |
| | (m, 1H), 6.37 (d, J = 8.0 Hz, 1H), 5.13 (td, J = 4.5, 13.0 Hz, 1H), 4.72-4.34 (m, |
| | 4H), 4.25-4.04 (m, 1H), 3.71-3.39 (m, 6H), 3.30 (s, 1H), 3.13-2.98 (m, |
| | 5H), 2.93-2.85 (m, 1H), 2.84-2.67 (m, 2H), 2.54-2.41 (m, 1H), 2.36-2.13 |
| | (m, 3H), 2.09-1.93 (m, 1H), 1.87-1.64 (m, 2H), 1.63-1.29 (m, 2H), 0.99 (d, |
| | J = 7.0 Hz, 3H) |
| II-64 | LCMS: calc. for C₄₅H₄₇Cl₂N₅O₄: 792.79, found: [M + H]⁺ 792.3. |
| | ¹HNMR (400 MHz, DMSO-d₆) δ = 10.95 (s, 1H), 9.97 (br s, 1H), 8.19 (s, 1H), |
| | 7.52 (d, J = 8.5 Hz, 1H), 7.22-7.16 (m, 3H), 7.08-7.03 (m, 2H), 6.88 (br d, |
| | J = 6.3 Hz, 2H), 6.80-6.76 (m, 2H), 6.74-6.70 (m, 1H), 6.29-6.24 (m, 2H), |
| | 5.05 (dd, J = 5.1, 13.2 Hz, 1H), 4.37-4.29 (m, 1H), 4.28-4.17 (m, 2H), 3.29 |
| | (br d, J = 3.5 Hz, 8H), 3.21-3.08 (m, 3H), 2.95-2.74 (m, 2H), 2.58 (br d, |
| | J = 19.1 Hz, 1H), 2.46-2.43 (m, 1H), 2.38 (br d, J = 4.5 Hz, 1H), 2.22 (br d, |
| | J = 7.3 Hz, 2H), 2.11-1.91 (m, 3H), 1.86 (br s, 1H), 1.77 (br d, J = 12.0 Hz, |
| | 2H), 1.64 (br s, 1H), 1.31-1.16 (m, 3H) |
| II-52 | LCMS: calc. for C₄₅H₄₇F₂N₅O₄: 722.38, found: [M + H]⁺ 723.4. |
| | ¹HNMR (400 MHz, DMSO-d6) δ = 10.95 (s, 1H), 9.13 (s, 1H), 8.14 (s, 1H), |
| | 7.52 (d, J = 8.6 Hz, 1H), 7.14-7.10 (m, 3H), 7.08-7.04 (m, 2H), 6.84-6.78 |
| | (m, 4H), 6.68-6.61 (m, 2H), 6.49 (dd, J = 2.4, 8.2 Hz, 1H), 6.29 (d, J = 8.0 Hz, |
| | 2H), 5.05 (dd, J = 5.1, 13.2 Hz, 1H), 4.36-4.30 (m, 1H), 4.24-4.17 (m, 2H), |
| | 3.05-2.83 (m, 5H), 2.61 (br s, 6H), 2.38 (br d, J = 4.5 Hz, 2H), 2.29 (br s, 1H), |
| | 2.21-2.04 (m, 3H), 2.00-1.92 (m, 1H), 1.86 (br d, J = 11.2 Hz, 2H), 1.71 (br |
| | d, J = 11.7 Hz, 3H), 1.54 (br d, J = 19.3 Hz, 2H), 1.41-1.26 (m, 2H), 1.04-0.91 |
| | (m, 2H) |
| II-53 | LCMS: calc. for C₄₅H₄₇F₂N₅O₄: 722.38, found: [M + H]⁺ 723.4. |
| | ¹HNMR (400 MHz, DMSO-d6) δ = 10.95 (s, 1H), 9.13 (s, 1H), 7.63-7.57 (m, |
| | 1H), 7.33-7.29 (m, 1H), 7.12 (br d, J = 3.8 Hz, 5H), 6.88 (br d, J = 7.4 Hz, 1H), |
| | 6.84-6.78 (m, 3H), 6.69-6.61 (m, 2H), 6.49 (br d, J = 8.0 Hz, 1H), 6.34-6.27 |
| | (m, 2H), 5.06 (br dd, J = 5.0, 12.9 Hz, 1H), 4.35 (br d, J = 16.9 Hz, 1H), 4.27- |
| | 4.18 (m, 2H), 3.04-2.86 (m, 6H), 2.61 (br s, 6H), 2.11 (br d, J = 12.3 Hz, 3H), |
| | 1.98 (br s, 2H), 1.87 (br d, J = 10.1 Hz, 2H), 1.72 (br s, 3H), 1.66-1.49 (m, |
| | 5H), 1.35 (br s, 1H), 1.04 (br s, 1H) |
| II-65 | LCMS: calc. for C₄₅H₄₈ClN₅O₄: 757.34, found: [M + H]⁺ 758.3. |
| | ¹HNMR (400 MHz, DMSO-d₆) δ = 10.96 (s, 1H), 9.85 (br s, 1H), 8.17 (s, 1H), |
| | 7.52 (d, J = 8.5 Hz, 1H), 7.20-7.11 (m, 3H), 7.09-7.02 (m, 2H), 6.85 (br d, |
| | J = 6.5 Hz, 2H), 6.78-6.72 (m, 1H), 6.71-6.65 (m, 1H), 6.55 (br d, J = 8.8 Hz, |
| | 2H), 6.20 (d, J = 8.8 Hz, 2H), 5.05 (dd, J = 4.9, 13.4 Hz, 1H), 4.38-4.28 (m, |
| | 1H), 4.25-4.15 (m, 2H), 3.52 (br d, J = 8.3 Hz, 2H), 3.28 (br s, 6H), 3.19-3.09 |
| | (m, 1H), 2.97-2.72 (m, 2H), 2.61 (br s, 3H), 2.48-2.44 (m, 3H), 2.43-2.36 |
| | (m, 1H), 2.19 (br d, J = 7.0 Hz, 2H), 2.11 (td, J = 6.1, 12.3 Hz, 1H), 2.00-1.91 |
| | (m, 1H), 1.87-1.71 (m, 3H), 1.64 (br s, 1H), 1.21-1.09 (m, 2H) |
| II-66 | LCMS: calc. for C44H48N4O5: 712.36, found: [M + H]⁺713.5. |
| | ¹H NMR (500 MHz, CD₃OD) δ = 8.46 (s, 1H), 7.66 (d, J = 8.5 Hz, 1H), 7.14- |
| | 7.09 (m, 5H), 6.81 (d, J = 6.5 Hz, 2H), 6.71-6.64 (m, 2H), 6.53-6.50 (m, 3H), |
| | 6.30 (d, J = 8.5 Hz, 2H), 5.10 (dd, J = 5.0, 13.3 Hz, 1H), 4.61 (s, 4H), 4.47-4.35 |
| | (m, 2H), 4.20 (d, J = 5.0 Hz, 1H), 3.87 (t, J = 6.0 Hz, 2H), 3.44 (s, 3H), 3.09- |
| | 2.92 (m, 3H), 2.87 (br d, J = 4.5 Hz, 4H), 2.81-2.75 (m, 1H), 2.46 (dq, J = 5.0, |
| | 13.1 Hz, 1H), 2.23-2.13 (m, 2H), 1.79-1.74 (m, 3H), 1.71-1.64 (m, 2H), |
| | 1.54-1.48 (m, 2H) |
| II-44 | LCMS: calc. for C₄₆H₅₁N₅O₄, found: [M + H]⁺ 738.5. |
| | ¹H NMR (400 MHz, DMSO-d₆) δ = 10.95 (s, 1H), 9.11 (s, 1H), 7.51 (d, J = 8.8 |
| | Hz, 1H), 7.18-7.09 (m, 3H), 7.07-6.97 (m, 2H), 6.84 (br d, J = 7.7 Hz, 2H), |
| | 6.68-6.58 (m, 2H), 6.56-6.46 (m, 3H), 6.20 (d, J = 8.5 Hz, 2H), 5.05 (dd, |
| | J = 5.0, 13.2 Hz, 1H), 4.37-4.29 (m, 1H), 4.26-4.16 (m, 1H), 4.13 (br d, J = 4.6 |
| | Hz, 1H), 3.55 (br s, 2H), 3.31 (s, 5H), 3.26 (br s, 2H), 3.05-2.84 (m, 3H), |
| | 2.69-2.65 (m, 2H), 2.38-2.26 (m, 2H), 2.15-1.86 (m, 4H), 1.70 (br d, |
| | J = 10.0 Hz, 2H), 1.41 (br s, 1H), 1.24 (s, 2H), 1.17 (br d, J = 11.6 Hz, 2H), 0.91 |
| | (d, J = 6.6 Hz, 3H) |
| II-67 | LCMS: calc. for C₄₆H₅₁N₅O₄, found: [M + H]⁺ 738.5. |
| | ¹H NMR (400 MHz, DMSO-d₆) δ = 10.95 (s, 1H), 9.11 (s, 1H), 7.52 (d, J = 8.8 |
| | Hz, 1H), 7.18-7.11 (m, 3H), 7.08-7.02 (m, 2H), 6.84 (br d, J = 7.5 Hz, 2H), |
| | 6.66-6.59 (m, 2H), 6.56-6.47 (m, 3H), 6.20 (d, J = 8.6 Hz, 2H), 5.05 (dd, |
| | J = 5.0, 13.2 Hz, 1H), 4.37-4.29 (m, 1H), 4.24-4.12 (m, 2H), 3.55 (br s, 2H), |
| | 3.31 (s, 3H), 3.26 (br s, 2H), 3.01-2.87 (m, 3H), 2.69-2.65 (m, 2H), 2.36- |
| | 2.28 (m, 2H), 2.14-1.94 (m, 3H), 1.70 (br d, J = 7.5 Hz, 2H), 1.43 (br d, J = 8.3 |
| | Hz, 1H), 1.27-1.12 (m, 3H), 0.91 (d, J = 6.4 Hz, 3H) |
| II-68 | LCMS: calc. for C44H48N4O5: 737.36, found: [M + H]⁺738.5. |
| | ¹H NMR (500 MHz, CD₃OD) δ = 8.15 (s, 1H), 7.66 (d, J = 8.5 Hz, 1H), 7.15- |
| | 7.06 (m, 5H), 6.84-6.78 (m, 2H), 6.70 (d, J = 8.5 Hz, 1H), 6.66-6.61 (m, 3H), |
| | 6.52 (dd, J = 2.5, 8.3 Hz, 1H), 6.32 (d, J = 8.5 Hz, 2H), 5.10 (dd, J = 5.0, 13.3 Hz, |
| | 1H), 4.56 (s, 2H), 4.46-4.35 (m, 2H), 4.18 (d, J = 5.0 Hz, 1H), 3.78 (d, J = 16.5 |
| | Hz, 4H), 3.59 (d, J = 10.5 Hz, 2H), 3.44-3.33 (m, 4H), 3.08-2.97 (m, 2H), |
| | 2.88-2.78 (m, 2H), 2.70 (t, J = 11.0 Hz, 2H), 2.46 (dq, J = 4.5, 13.2 Hz, 1H), |
| | 2.28-2.10 (m, 2H), 1.89-1.73 (m, 5H) |
| II-70 | LCMS: calc. for C46H51N5O4, found: [M + H]⁺ 738.5. |
| | ¹H NMR (400 MHz, DMSO-d₆)δ = 7.85 (s, 1H), 7.66 (d, J = 8.9 Hz, 1H), 7.10- |
| | 7.05 (m, 3H), 6.92 (br d, J = 10.1 Hz, 1H), 6.80 (s, 1H), 6.78-6.72 (m, 3H), |
| | 6.63 (s, 1H), 6.51 (br d, J = 5.7 Hz, 2H), 6.22 (br d, J = 8.6 Hz, 2H), 5.13 (dd, |
| | J = 5.5, 13.2 Hz, 1H), 4.37-4.30 (m, 1H), 4.22-4.12 (m, 2H), 3.51 (br d, |
| | J = 11.7 Hz, 2H), 3.33-3.09 (m, 5H), 3.01-2.91 (m, 2H), 2.86-2.62 (m, 4H), |
| | 2.47 (br s, 3H), 2.33-2.18 (m, 2H), 2.17-2.09 (m, 2H), 1.71 (br s, 2H), 1.31- |
| | 1.13 (m, 5H), 0.91 (br s, 3H) |
| II-73 | LC-MS (ESI+) m/z: 774.2 (M + H)+ |
| | ¹H NMR (400 MHz, DMSO-d₆) δ = 10.97-10.93 (m, 1H), 9.15-9.10 (m, |
| | 1H), 8.16-8.13 (m, 0.200H), 7.57-7.46 (m, 1H), 7.09-7.02 (m, 2H), 7.01- |
| | 6.87 (m, 1H), 6.67-6.54 (m, 5H), 6.52-6.46 (m, 1H), 6.33-6.25 (m, 2H), |
| | 5.11-4.99 (m, 1H), 4.39-4.29 (m, 1H), 4.25-4.16 (m, 1H), 4.10-4.00 (m, |
| | 1H), 3.60-3.50 (m, 2H), 3.32-3.11 (m, 10H), 2.96-2.84 (m, 3H), 2.63-2.54 |
| | (m, 2H), 2.48-2.38 (m, 4H), 2.37-2.29 (m, 1H), 2.24-2.17 (m, 2H), 2.01- |
| | 1.92 (m, 1H), 1.68 (br d, J = 14.9 Hz, 4H), 1.24-1.14 (m, 2H). |
| II-74 | LC-MS (ESI+) m/z: 774.2 (M + H)+ |
| | ¹H NMR (400 MHz, DMSO-d₆) δ = 11.00-10.91 (m, 1H), 9.20-9.06 (m, |
| | 1H), 8.20-8.11 (m, 0.270H), 7.57-7.47 (m, 1H), 7.09-7.03 (m, 2H), 7.01- |
| | 6.89 (m, 1H), 6.67-6.55 (m, 5H), 6.53-6.46 (m, 1H), 6.33-6.26 (m, 2H), |
| | 5.09-5.01 (m, 1H), 4.37-4.29 (m, 1H), 4.25-4.17 (m, 1H), 4.09-4.02 (m, |
| | 1H), 3.61-3.44 (m, 4H), 3.29 (br s, 8H), 2.95-2.83 (m, 3H), 2.63-2.54 (m, |
| | 2H), 2.48-2.43 (m, 3H), 2.43-2.30 (m, 2H), 2.24-2.18 (m, 2H), 2.01-1.92 |
| | (m, 1H), 1.81-1.62 (m, 4H), 1.25-1.13 (m, 2H). |
| II-75 | LC-MS (ESI⁺) m/z: 774.3 (M + H)⁺. |
| | ¹H NMR (400 MHz, METHANOL-d₄) δ ppm: 8.28 (s, 1 H), 7.66 (d, J = 9.06 |
| | Hz, 2 H), 7.09-7.14 (m, 2 H), 6.94 (dd, J = 10.37, 6.44 Hz, 1 H), 6.64-6.70 |
| | (m, 3 H), 6.52 (dd, J = 8.34, 2.62 Hz, 1 H), 6.41 (d, J = 8.70 Hz, 2 H), 5.98 (dd, |
| | J = 10.61, 6.08 Hz, 1 H), 5.10 (dd, J = 13.29, 5.19 Hz, 1 H), 4.35-4.45 (m, 2 |
| | H), 4.24 (d, J = 5.13 Hz, 1 H), 3.53-3.60 (m, 3 H), 3.48 (br s, 4 H), 2.94- |
| | 3.04 (m, 6 H), 2.84-2.92 (m, 1 H), 2.73-2.81 (m, 1 H), 2.59-2.69 (m, 4 H) |
| | 2.45 (qd, J = 13.21, 4.71 Hz, 1 H), 2.16 (br s, 5 H), 1.87 (br d, J = 10.25 Hz, 3 |
| | H), 1.65-1.72 (m, 1 H), 1.32-1.45 (m, 2 H). |
| II-76 | LC-MS (ESI+) m/z: 774.3 (M + H)⁺. |
| | ¹H NMR (400 MHz, METHANOL-d₄) δ ppm: 7.66 (d, J = 9.18 Hz, 1 H), 7.37- |
| | 7.88 (m, 1 H), 7.09-7.15 (m, 2 H), 6.94 (dd, J = 10.31, 6.38 Hz, 1 H), 6.64- |
| | 6.70 (m, 3 H), 6.52 (dd, J = 8.29, 2.32 Hz, 1 H), 6.41 (d, J = 8.58 Hz, 2 H), |
| | 5.98 (dd, J = 10.49, 6.08 Hz, 1 H), 5.10 (dd, J = 13.29, 5.07 Hz, 1 H), 4.35- |
| | 4.47 (m, 2 H), 4.24 (d, J = 5.01 Hz, 1 H), 3.53-3.61 (m, 3 H), 3.49 (br s, 4 |
| | H), 3.00 (br d, J = 4.53 Hz, 6 H), 2.84-2.94 (m, 1 H), 2.73-2.81 (m, 1 H), |
| | 2.59-2.69 (m, 4 H), 2.46 (qd, J = 13.17, 4.47 Hz, 1 H), 2.11-2.20 (m, 5 H), |
| | 1.87 (br d, J = 10.61 Hz, 3 H), 1.69 (br dd, J = 12.76, 2.15 Hz, 1 H), 1.32-1.44 |
| | (m, 2 H). |
| II-77 | ¹H NMR (400 MHz, DMSO-d₆) δ ppm 10.95 (s, 1 H) 8.97-9.14 (m, 1 H) 8.15 |
| | (s, 0.491 H) 7.98 (d, J = 2.74 Hz, 1 H) 7.52 (d, J = 8.58 Hz, 1 H) 6.99-7.15 (m, |
| | 5 H) 6.93 (dd, J = 8.70, 2.86 Hz, 1 H) 6.83 (d, J = 6.80 Hz, 2 H) 6.53-6.66 (m, 2 |
| | H) 6.35-6.47 (m, 2 H) 5.05 (dd, J = 13.23, 5.13 Hz, 1 H) 4.28-4.40 (m, 1 H) |
| | 4.15-4.26 (m, 2 H) 3.53-3.62 (m, 3 H) 3.23-3.30 (m, 8 H) 2.84-3.04 (m, 4 |
| | H) 2.63-2.80 (m, 2 H) 2.28-2.46 (m, 2 H) 2.14-2.25 (m, 2 H) 1.88-2.04 |
| | (m, 1 H) 1.61-1.82 (m, 4 H) 1.08-1.26 (m, 2 H) |
| II-78 | LC-MS (ESI⁺) m/z: 725.2 (M + H)⁺. |
| | ¹H NMR (400 MHz, DMSO-d₆) δ ppm 10.95 (s, 1 H) 9.00-9.08 (m, 1 H) 8.15 |
| | (s, 0.223 H) 7.98 (d, J = 2.86 Hz, 1 H) 7.47-7.56 (m, 1 H) 7.00-7.15 (m, 5 H) |
| | 6.93 (dd, J = 8.64, 2.92 Hz, 1 H) 6.83 (d, J = 6.79 Hz, 2 H) 6.54-6.66 (m, 2 H) |
| | 6.34-6.48 (m, 2 H) 5.05 (dd, J = 13.29, 5.07 Hz, 1 H) 4.28-4.37 (m, 1 H) 4.17- |
| | 4.28 (m, 2 H) 3.57 (br d, J = 4.77 Hz, 2 H) 3.28 (br s, 8 H) 2.99 (br s, 3 H) |
| | 2.56-2.73 (m, 4 H) 2.26-2.47 (m, 2 H) 2.20 (br d, J = 7.03 Hz, 2 H) 1.91- |
| | 2.01 (m, 1 H) 1.61-1.81 (m, 4 H) 1.09-1.25 (m, 2 H) |
| II-79 | LC-MS (ESI⁺) m/z: 725.3 (M + H)⁺. |
| | ¹H NMR (400 MHz, DMSO-d₆) δ ppm 10.95 (s, 1 H) 9.21 (s, 1 H) 8.11 (s, 0.248 |
| | H) 7.52 (d, J = 8.53 Hz, 1 H) 7.13-7.23 (m, 3 H) 7.03-7.09 (m, 3 H) 6.87-6.93 |
| | (m, 2 H) 6.67 (d, J = 8.28 Hz, 1 H) 6.61 (d, J = 2.01 Hz, 1 H) 6.53 (br d, J = 2.26 |
| | Hz, 2 H) 6.43-6.47 (m, 1 H) 5.05 (dd, J = 13.30, 5.02 Hz, 1 H) 4.17-4.37 (m, |
| | 2 H) 4.13 (br d, J = 5.02 Hz, 1 H) 4.03-4.11 (m, 2 H) 3.28 (br s, 8 H) 2.84-3.04 |
| | (m, 3 H) 2.56-2.68 (m, 3 H) 2.28-2.43 (m, 2 H) 2.18 (br d, J = 6.53 Hz, 2 H) |
| | 2.00-2.08 (m, 1 H) 1.92-1.98 (m, 1 H) 1.67-1.80 (m, 4 H) 0.96-1.09 (m, 2 |
| | H) |
| II-80 | LC-MS (ESI⁺) m/z: 725.2 (M + H)⁺. |
| | ¹H NMR (400 MHz, DMSO-d₆) δ ppm 10.92-10.98 (m, 1 H) 9.11-9.20 (m, 1 |
| | H) 8.16 (s, 0.298 H) 7.49-7.55 (m, 1 H) 7.11-7.23 (m, 3 H) 7.02-7.09 (m, 3 |
| | H) 6.87-6.93 (m, 2 H) 6.64-6.70 (m, 1 H) 6.58-6.63 (m, 1 H) 6.48-6.55 (m, |
| | 2 H) 6.42-6.47 (m, 1 H) 5.05 (dd, J = 13.23, 5.13 Hz, 1 H) 4.17-4.36 (m, 2 H) |
| | 4.13 (br d, J = 4.89 Hz, 1 H) 4.07 (br d, J = 4.89 Hz, 2 H) 3.28 (br d, J = 2.98 Hz, 8 |
| | H) 2.85-3.00 (m, 3 H) 2.55-2.68 (m, 4 H) 2.32-2.39 (m, 1 H) 2.15-2.20 (m, |
| | 2 H) 2.01-2.09 (m, 1 H) 1.91-1.99 (m, 1 H) 1.67-1.81 (m, 4 H) 0.95-1.10 |
| | (m, 2 H) |
| II-109 | LCMS: calc. for C₄₅H₄₈FN₅O₄: 741.37, found: [M + H]⁺742.4. |
| | ¹H NMR (400 MHz, DMSO-d₆) δ = 10.94 (br s, 1 H) 9.12 (s, 1 H) 7.52 (d, J = 8.53 |
| | Hz, 1 H) 7.03-7.09 (m, 2 H) 6.93-7.01 (m, 2 H) 6.81-6.89 (m, 2 H) 6.64 (d, |
| | J = 8.53 Hz, 1 H) 6.54-6.61 (m, 3 H) 6.48 (dd, J = 8.28, 2.26 Hz, 1 H) 6.22 (d, |
| | J = 8.78 Hz, 2 H) 5.05 (dd, J = 13.30, 5.02 Hz, 1 H) 4.29-4.37 (m, 1 H) 4.17- |
| | 4.26 (m, 1 H) 4.11 (d, J = 4.77 Hz, 1 H) 3.53 (br d, J = 12.05 Hz, 2 H) 3.32 (br s, |
| | 2 H) 3.28 (br s, 4 H) 2.83-3.02 (m, 3 H) 2.55-2.62 (m, 1 H) 2.45-2.50 (m, 5 |
| | H) 2.32-2.39 (m, 1 H) 2.19 (br d, J = 7.03 Hz, 2 H) 2.08 (br dd, J = 11.54, 6.78 |
| | Hz, 1 H) 1.93-2.00 (m, 1 H) 1.76 (br d, J = 11.80 Hz, 2 H) 1.68 (br d, J = 10.04 |
| | Hz, 2 H) 1.11-1.24 (m, 2 H) |
| II-104 | LCMS: calc. for C₄₆H₄₈N₄O₄: 720.37, found: [M + H]⁺ 721.5 |
| | ¹H NMR (400 MHz, METHANOL-d₄) δ: ppm 7.72-7.79 (m, 1 H), 7.59- |
| | 7.67 (m, 2 H), 7.08-7.13 (m, 3 H), 6.80 (m, 2 H), 6.69 (d, J = 8.40 Hz, 1 |
| | H), 6.60-6.66 (m, 3 H), 6.52 (m, 1 H), 6.31 (d, J = 8.40 Hz, 3 H), 5.15 (m, 1 |
| | H), 4.42-4.57 (m, 2 H), 4.18 (d, J = 4.80 Hz, 1 H), 3.53 (d, J = 10.40 Hz, 2 |
| | H), 3.26 (s, 2 H), 2.96-3.05 (m, 2 H), 2.86-2.94 (m, 1 H), 2.76-2.82 (m, 2 |
| | H), 2.67 (br s, 2 H), 2.43-2.63 (m, 5 H), 2.16-2.25 (m, 2 H), 1.88 (d, |
| | J = 12.40 Hz, 2 H), 1.76 (m, 2 H), 1.28-1.41 (m, 4 H). |
| II-105 | LCMS: calc. for C₄₆H₅₀N₄O₄: 722.38, found: [M + H]⁺ 723.4 |
| | ¹H NMR (400 MHz, METHANOL-d₄) δ: ppm 7.73 (d, J = 8.00 Hz, 1 H), 7.48 |
| | (s, 1 H), 7.43 (d, J = 7.60 Hz, 1 H), 7.07-7.13 (m, 3 H), 6.80 (m, 2 H), 6.69 (d, |
| | J = 8.40 Hz, 1 H), 6.61-6.66 (m, 3 H), 6.51 (m, 1 H), 6.31 (d, J = 8.40 Hz, 2 H), |
| | 5.11-5.19 (m, 1 H), 4.40-4.52 (m, 2 H), 4.18 (d, J = 4.80 Hz, 1 H), 3.50-3.55 |
| | (m, 2 H), 3.10 (d, J = 11.20 Hz, 2 H), 2.96-3.05 (m, 2 H), 2.85-2.94 (m, 1 H), |
| | 2.69-2.83 (m, 2 H), 2.54-2.61 (m, 2 H), 2.49 (m, 1 H), 2.30-2.35 (m, 2 H), |
| | 2.21-2.28 (m, 1 H), 2.11-2.20 (m, 3 H), 1.83-1.90 (m, 6 H), 1.69-1.79 (m, |
| | 2 H), 1.29-1.40 (m, 3 H). |
| II-110 | LCMS: calc. for C₄₅H₄₈FN₅O₄: 741.37, found: [M + H]⁺742.3. |
| | ¹H NMR 1H NMR (400 MHz, DMSO-d₆) δ = 10.77-11.18 (m, 1 H) 9.14 (br s, |
| | 1 H) 7.52 (d, J = 8.53 Hz, 1 H) 7.02-7.09 (m, 2 H) 6.93-7.00 (m, 2 H) 6.82- |
| | 6.89 (m, 2 H) 6.54-6.66 (m, 4 H) 6.48 (dd, J = 8.28, 2.26 Hz, 1 H) 6.22 (d, |
| | J = 8.28 Hz, 2 H) 5.05 (dd, J = 13.30, 5.02 Hz, 1 H) 4.27-4.38 (m, 1 H) 4.16- |
| | 4.25 (m, 1 H) 4.11 (br d, J = 4.77 Hz, 1 H) 3.52 (br d, J = 10.79 Hz, 2 H) 3.32 (br |
| | s, 2 H) 3.28 (br s, 4 H) 2.85-3.02 (m, 3 H) 2.58 (br d, J = 17.07 Hz, 2 H) 2.46- |
| | 2.49 (m, 4 H) 2.36 (br dd, J = 13.55, 4.27 Hz, 1 H) 2.19 (br d, J = 6.78 Hz, 2 H) |
| | 2.01-2.13 (m, 1 H) 1.92-1.99 (m, 1 H) 1.76 (br d, J = 12.05 Hz, 2 H) 1.68 (br |
| | d, J = 10.04 Hz, 2 H) 1.12-1.24 (m, 2 H) |
| II-106 | LCMS: calc. for C₄₆H₅₀N₄O₅: 738.91, found: [M + H]⁺ 739.4. |
| | ¹H NMR (400 MHz, DMSO-d₆) δ = 10.99 (s, 1H), 9.11 (s, 1H), 8.16-8.10 (m, |
| | 1H), 7.62 (d, J = 7.6 Hz, 1H), 7.47 (s, 1H), 7.37 (br d, J = 8.0 Hz, 1H), 7.13 (br |
| | d, J = 2.0 Hz, 2H), 6.83 (br d, J = 6.7 Hz, 2H), 6.63 (d, J = 8.5 Hz, 1H), 6.59 (d, |
| | J = 1.4 Hz, 1H), 6.53 (br d, J = 8.6 Hz, 2H), 6.47 (br d, J = 7.9 Hz, 1H), 6.19 (d, |
| | J = 8.5 Hz, 2H), 5.10 (dd, J = 4.8, 13.5 Hz, 1H), 4.77-4.58 (m, 1H), 4.43-4.38 |
| | (m, 1H), 4.30-4.24 (m, 1H), 4.12 (br d, J = 4.3 Hz, 1H), 3.70-3.64 (m, 1H), |
| | 3.52 (br d, J = 11.4 Hz, 2H), 3.27-3.24 (m, 1H), 3.09-3.03 (m, 1H), 2.98- |
| | 2.85 (m, 5H), 2.61 (br s, 1H), 2.56 (br d, J = 1.5 Hz, 1H), 2.41 (br d, J = 2.1 Hz, |
| | 1H), 2.39-2.34 (m, 1H), 2.29-2.24 (m, 1H), 2.09 (br dd, J = 6.3, 12.3 Hz, 1H), |
| | 2.02-1.94 (m, 2H), 1.72 (br s, 6H), 1.65-1.58 (m, 1H), 1.27-1.10 (m, 3H) |
| II-107 | LCMS: calc. for C₄₆H₅₀N₄O₅: 738.93, found: [M + H]⁺ 739.4. |
| | ¹H NMR: ¹H NMR (400 MHz, DMSO-d₆) δ = 10.99 (s, 1H), 9.11 (s, 1H), 8.16- |
| | 8.10 (m, 1H), 7.62 (d, J = 7.6 Hz, 1H), 7.47 (s, 1H), 7.37 (br d, J = 8.0 Hz, |
| | 1H), 7.13 (br d, J = 2.0 Hz, 2H), 6.83 (br d, J = 6.7 Hz, 2H), 6.63 (d, J = 8.5 |
| | Hz, 1H), 6.59 (d, J = 1.4 Hz, 1H), 6.53 (br d, J = 8.6 Hz, 2H), 6.47 (br d, J = |
| | 7.9 Hz, 1H), 6.19 (d, J = 8.5 Hz, 2H), 5.10 (dd, J = 4.8, 13.5 Hz, 1H), 4.77- |
| | 4.58 (m, 1H), 4.43-4.38 (m, 1H), 4.30-4.24 (m, 1H), 4.12 (br d, J = 4.3 Hz, |
| | 1H), 3.70-3.64 (m, 1H), 3.52 (br d, J = 11.4 Hz, 2H), 3.27-3.24 (m, 1H), |
| | 3.09-3.03 (m, 1H), 2.98-2.85 (m, 5H), 2.61 (br s, 1H), 2.56 (br d, J = 1.5 |
| | Hz, 1H), 2.41 (br d, J = 2.1 Hz, 1H), 2.39-2.34 (m, 1H), 2.29-2.24 (m, 1H), |
| | 2.09 (br dd, J = 6.3, 12.3 Hz, 1H), 2.02-1.94 (m, 2H), 1.72 (br s, 6H), 1.65- |
| | 1.58 (m, 1H), 1.27-1.10 (m, 3H) |
| II-88 | LC-MS (ESI⁺) m/z: 741.3 (M + H)⁺. |
| | ¹H NMR (400 MHz, METHANOL-d4) δ ppm 7.76 (d, J = 7.87 Hz, 1 H), 7.54 |
| | (s, 1 H), 7.47 (br d, J = 7.63 Hz, 1 H), 7.08-7.14 (m, 3 H), 6.78-6.83 (m, 2 |
| | H), 6.69 (d, J = 8.34 Hz, 1 H), 6.61-6.66 (m, 3 H), 6.51 (dd, J = 8.29, 2.44 |
| | Hz, 1 H), 6.31 (d, J = 8.58 Hz, 2 H), 5.15 (dd, J = 13.35, 5.13 Hz, 1 H), 4.66- |
| | 4.81 (m, 1 H), 4.43-4.53 (m, 2 H), 4.18 (d, J = 5.13 Hz, 1 H), 3.52 (br d, J = |
| | 9.78 Hz, 2 H), 2.70-3.16 (m, 7 H), 2.06-2.61 (m, 10 H), 1.83-1.92 (m, 4 |
| | H), 1.66-1.79 (m, 2 H), 1.28-1.35 (m, 2 H) |
| II-89 | LCMS: calc. for C46H49FN4O4: 740.92, found: [M + H]⁺ 741.3. |
| | ¹H NMR (400 MHz, METHANOL-d₄) δ ppm 7.76 (d, J = 7.87 Hz, 1 H), 7.54 (s, |
| | 1 H), 7.47 (br d, J = 7.87 Hz, 1 H), 7.09-7.13 (m, 3 H), 6.79-6.82 (m, 2 H), |
| | 6.69 (d, J = 8.34 Hz, 1 H), 6.62-6.66 (m, 3 H), 6.52 (dd, J = 8.34, 2.50 Hz, 1 H), |
| | 6.31 (d, J = 8.58 Hz, 2 H), 5.15 (dd, J = 13.29, 5.19 Hz, 1 H), 4.65-4.80 (m, 1 H), |
| | 4.43-4.54 (m, 2 H), 4.18 (d, J = 4.77 Hz, 1 H), 3.52 (br d, J = 9.18 Hz, 2 H), 2.74- |
| | 3.14 (m, 7 H), 2.44-2.61 (m, 3 H), 2.04-2.39 (m, 7 H), 1.84-1.92 (m, 4 H), |
| | 1.67-1.80 (m, 2 H), 1.29-1.36 (m, 2 H) |
| II-91 | LCMS: calc. for C₄₆H₄₈F₂N₄O₄: 758.91, found: [M + H]⁺ 759.4. |
| | ¹H NMR (400 MHz, DMSO-d₆) δ ppm 10.99 (s, 1 H), 9.10 (s, 1 H), 7.69 (br d, |
| | J = 7.75 Hz, 1 H), 7.54-7.60 (m, 1 H), 7.46 (br d, J = 8.11 Hz, 1 H), 7.09-7.19 |
| | (m, 3 H), 6.83 (br d, J = 6.91 Hz, 2 H), 6.59-6.66 (m, 2 H), 6.46-6.56 (m, 3 |
| | H), 6.20 (br d, J = 8.11 Hz, 2 H), 5.11 (br dd, J = 13.23, 4.77 Hz, 1 H), 4.28- |
| | 4.49 (m, 2 H), 4.13 (br d, J = 4.65 Hz, 1 H), 3.52 (br d, J = 10.97 Hz, 2 H), 3.14- |
| | 3.27 (m, 2 H), 2.87-3.00 (m, 4 H), 2.60 (br d, J = 17.40 Hz, 2 H), 2.19-2.48 |
| | (m, 6 H), 1.95-2.18 (m, 4 H), 1.62-1.84 (m, 5 H), 1.09-1.22 (m, 2 H). |
| II-92 | LCMS: calc. for C46H48F2N4O4: 758.91, found: [M + H]⁺ 759.4. |
| | ¹H NMR (400 MHz, DMSO-d6) δ ppm 10.98 (br s, 1 H) 9.09 (s, 1 H) 7.69 (d, |
| | J = 7.87 Hz, 1 H) 7.57 (s, 1 H) 7.46 (d, J = 7.87 Hz, 1 H) 7.10-7.16 (m, 3 H) |
| | 6.83 (br d, J = 6.68 Hz, 2 H) 6.59-6.66 (m, 2 H) 6.46-6.55 (m, 3 H) 6.20 (d, |
| | J = 8.58 Hz, 2 H) 5.11 (dd, J = 13.11, 4.77 Hz, 1 H) 4.29-4.48 (m, 2 H) 4.13 (d, |
| | J = 4.89 Hz, 1 H) 3.50 (br d, J = 4.05 Hz, 2 H) 3.14-3.27 (m, 2 H) 2.87-2.99 |
| | (m, 4 H) 2.53-2.69 (m, 2 H) 1.97-2.47 (m, 10 H) 1.62-1.83 (m, 5 H) 1.10- |
| | 1.20 (m, 2 H) |
| II-100 | LCMS: calc. for C₄₅H₄₉N₅O₄: 723.9, found: [M + H]⁺ 724.6. |
| | ¹HNMR (400 MHz, DMSO-d₆): δ 10.94 (s, 1H), 9.10 (s, 1H), 8.14 (s, 1H), 7.51 |
| | (d, J = 8.6 Hz, 1H), 7.18-7.02 (m, 5H), 6.83 (d, J = 6.6 Hz, 2H), 6.67-6.58 |
| | (m, 2H), 6.56-6.45 (m, 3H), 6.20 (d, J = 8.6 Hz, 2H), 5.04 (dd, J = 5.1, 13.4 |
| | Hz, 1H), 4.38-4.16 (m, 2H), 4.12 (d, J = 4.9 Hz, 1H), 3.51 (d, J = 11.2 Hz, 2H), |
| | 3.27 (s, 6H), 3.05-2.84 (m, 3H), 2.63-2.56 (m, 1H), 2.59 (s, 6H), 2.38 (d, J = |
| | 4.5 Hz, 1H), 2.19 (s, 2H), 2.16-2.03 (m, 1H), 2.01-1.90 (m, 1H), 1.79-1.58 |
| | (m, 1H), 1.81-1.57 (m, 3H), 1.22-1.09 (m, 2H). |
| II-81 | LCMS: calc. for C₄₅H₄₈FN₅O₄, found: [M + H]⁺742.4. |
| | ¹H NMR (400 MHz, DMSO-d₆) δ = 10.94 (s, 1 H) 9.18 (br s, 1 H) 8.18 (s, |
| | 0.302 H) 7.51 (d, J = 8.70 Hz, 1 H) 7.12-7.20 (m, 3 H) 7.02-7.08 (m, 2 H) |
| | 6.86 (br d, J = 6.68 Hz, 2 H) 6.60-6.68 (m, 3 H) 6.49 (dd, J = 8.23, 2.50 Hz, 1 |
| | H) 6.09 (br d, J = 8.34 Hz, 1 H) 5.97 (br d, J = 14.19 Hz, 1 H) 5.04 (dd, J = 13.29, |
| | 5.07 Hz, 1H) 4.28-4.37 (m, 1 H) 4.15-4.23 (m, 2 H) 3.14-3.29 (m, 12 H) |
| | 2.83-3.04 (m, 3 H) 2.54-2.62 (m, 1 H) 2.33-2.42 (m, 2 H) 2.20 (br d, |
| | J = 7.03 Hz, 2 H) 2.06 (br dd, J = 12.10, 6.14 Hz, 1 H) 1.92-1.99 (m, 1 H) 1.69- |
| | 1.80 (m, 3 H) 1.62 (br s, 1 H) 1.16-1.26 (m, 2 H) |
| II-82 | LCMS: calc. for C₄₅H₄₈FN₅O₄, found: [M + H]⁺742.4. |
| | ¹H NMR 1H NMR (400 MHz, DMSO-d₆) δ = 10.95 (s, 1 H) 9.17 (br s, 1 H) |
| | 8.18 (s, 0.245 H) 7.51 (d, J = 8.70 Hz, 1 H) 7.12-7.20 (m, 3 H) 7.02-7.08 (m, |
| | 2 H) 6.86 (br d, J = 6.68 Hz, 2 H) 6.60-6.68 (m, 3 H) 6.49 (dd, J = 8.29, 2.32 |
| | Hz, 1 H) 6.09 (br d, J = 8.34 Hz, 1 H) 5.93-6.00 (m, 1 H) 5.04 (dd, J = 13.23, |
| | 5.13 Hz, 1 H) 4.29-4.36 (m, 1 H) 4.16-4.23 (m, 2 H) 3.14-3.29 (m, 11 H) |
| | 2.84-3.03 (m, 3 H) 2.53-2.62 (m, 2 H) 2.33-2.43 (m, 2 H) 2.20 (br d, |
| | J = 7.03 Hz, 2 H) 2.06 (br dd, J = 12.16, 6.20 Hz, 1 H) 1.91-1.99 (m, 1 H) 1.75 |
| | (br d, J = 11.56 Hz, 3 H) 1.62 (br s, 1 H) 1.16-1.26 (m, 2 H) |
| II-83 | LCMS: calc. for C₄₅H₅₇F₂N₅O₄: 759.90, found: [M + H]⁺ 760.2. |
| | ¹H NMR (400 MHz, DMSO-d₆) δ = 10.95 (s, 1H), 9.25 (s, 1H), 8.14 (s, 0.118H), |
| | 7.54 (br d, J = 8.0 Hz, 1H), 7.25-7.14 (m, 3H), 7.09 (br s, 2H), 6.91 (br d, J = |
| | 7.0 Hz, 2H), 6.69 (d, J = 8.5 Hz, 1H), 6.63 (s, 1H), 6.53 (br d, J = 8.0 Hz, 1H), |
| | 5.88 (br d, J = 11.0 Hz, 2H), 5.05 (dd, J = 5.1, 13.2 Hz, 1H), 4.38-4.29(m, 1H), |
| | 4.25-4.16 (m, 2H), 3.33-3.22 (m, 7H), 3.07-2.84 (m, 8H), 2.76-2.65 (m, |
| | 1H), 2.61 (br s, 1H), 2.57 (br s, 1H), 2.38 (br d, J = 4.5 Hz, 1H), 2.37-2.31 (m, |
| | 1H), 2.07-1.92 (m, 2H), 1.73 (br d, J = 9.5 Hz, 4H), 1.26-1.12 (m, 2H) |
| II-84 | LCMS: calc. for C₄₅H₅₇F₂N₅O₄: 759.90, found: [M + H]+ 760.2. |
| | ¹H NMR (400 MHz, DMSO-d₆) δ = 10.95 (s, 1H), 9.24 (s, 1H), 8.14 (s, 0.276H), |
| | 7.52 (d, J = 8.5 Hz, 1H), 7.24-7.15 (m, 3H), 7.09-7.03 (m, 2H), 6.90 (brd, J = |
| | 7.3 Hz, 2H), 6.69 (d, J = 8.3 Hz, 1H), 6.62 (s, 1H), 6.52 (dd, J = 2.1, 8.2 Hz, |
| | 1H), 5.88 (d, J = 10.8 Hz, 2H), 5.05 (dd, J = 4.9, 13.2 Hz, 1H), 4.36-4.29(m, |
| | 1H), 4.25-4.17 (m, 2H), 3.28 (br s, 7H), 3.04-2.84 (m, 8H), 2.63-2.55 (m, |
| | 1H), 2.44-2.29 (m, 2H), 2.21 (br d, J = 6.8 Hz, 2H), 2.06-1.92 (m, 2H), 1.72 |
| | (br d, J = 12.0 Hz, 3H), 1.67-1.58 (m, 1H), 1.24-1.11 (m, 2H) |
| II-85 | LC-MS (ESI+) m/z: 742.2 (M + H)+ |
| | ¹H NMR (400 MHz, DMSO-d₆) δ = 11.04-10.87 (m, 1H), 9.36-8.98 (m, |
| | 1H), 8.19-8.12 (m, 0.350H), 7.58-7.43 (m, 1H), 7.20-6.95 (m, 5H), 6.90- |
| | 6.72 (m, 2H), 6.66-6.57 (m, 2H), 6.56-6.43 (m, 3H), 6.32-6.21 (m, 1H), |
| | 5.11-4.99 (m, 1H), 4.50-4.41 (m, 1H), 4.37-4.28 (m, 1H), 4.25-4.15 (m, |
| | 1H), 3.51 (m, 3H), 3.37-3.18 (m, 8H), 3.02-2.84 (m, 3H), 2.64-2.53 (m, |
| | 3H), 2.40-2.30 (m, 1H), 2.27-2.10 (m, 3H), 2.02-1.90 (m, 1H), 1.82-1.59 |
| | (m, 4H), 1.22-1.05 (m, 2H) |
| II-86 | LC-MS (ESI+) m/z: 742.3 (M + H)⁺ |
| | ¹H NMR (400 MHz, DMSO-d₆) δ = 11.00-10.91 (m, 1H), 9.25-9.06 (m, |
| | 1H), 8.17-8.13 (m, 0.262H), 7.58-7.47 (m, 1H), 7.17-6.99 (m, 5H), 6.87- |
| | 6.76 (m, 2H), 6.66-6.57 (m, 2H), 6.56-6.43 (m, 3H), 6.31-6.22 (m, 1H), |
| | 5.12-4.99 (m, 1H), 4.49-4.41 (m, 1H), 4.37-4.28 (m, 1H), 4.25-4.16 (m, |
| | 1H), 3.66-3.48 (m, 3H), 3.34-3.16 (m, 8H), 3.02-2.84 (m, 3H), 2.63-2.53 |
| | (m, 3H), 2.40-2.32 (m, 1H), 2.25-2.12 (m, 3H), 2.01-1.91 (m, 1H), 1.80- |
| | 1.61 (m, 4H), 1.22-1.07 (m, 2H). |

### Example A1—Nano Glo HiBiT Lytic Degradation Assay

Selected compounds of the present disclosure were tested in a Nano Glo HiBiT Lytic Degradation Assay to measure the level of ERα protein degradation in MCF7 cells.

TABLE 3
Materials Used.

| Materials | Vendor | Catalog number |
|---|---|---|
| Eagle's Minimum Essential Medium (EMEM) | Corning | 10009CV |
| Fetal Bovine Serum (FBS) | VWR | 89510-194 |
| Human Recombinant Insulin | Gibco | 12585-014 |
| 384-Well, Cell Culture-Treated, Flat-Bottom Microplate | Corning | 3570 |
| Nano Glo HiBiT Lytic Detection System | Promega | N3050 |
| ESR1_HiBiT KI MCF7 cells | Promega | CS302392 |

TABLE 4
Instruments Used.

| Instrument | Vendor |
|---|---|
| Vi-Cell | Beckman Coulter |
| MultiFlo Multimode Dispenser | BioTek |
| Echo Acoustic Liquid Handler | Beckman Coulter |
| EnVision 2105 Multimode Plate Reader | Perkin Elmer |

TABLE 5
Cell Line Information.

| Cell line | Growth Media |
|---|---|
| ESR1_HiBiT KI MCF7 | EMEM + 10% FBS + 0.01 mg/mL human recombinant insulin |

Cell culture: ESR1_HiBiT KI MCF7 cells were maintained in growth media without antibiotics. Cells were maintained in exponential growth phase, between 40% and 90% confluency.

Cell seeding: Cells were counted using a Vi-Cell and seeded in a 384-well flat-bottom microplate (cell culture-treated) at 4000 cells/well in in 25 μL of growth medium using a BioTek MultiFlo Multimode Dispenser. Plates were incubated for 24 h at 37° C., 5% CO2, >95% relative humidity.

Compound preparation: Compounds were screened as 14-point dose response curves 1:3 dilutions. DMSO is used as the vehicle control and all wells were normalized to the same final DMSO concentration (0.1% v/v). Compounds were dosed using an Echo Acoustic Liquid Handler, 25 nL of 1000× (10 mM top concentration) DMSO stock/well. Cells were incubated with compounds for 6 hours at 37° C., 5% CO2, >95% relative humidity. Each compound was run in duplicate.

Nano Glo HiBiT Lytic Degradation Assay: HiBiT-tagged ERα protein levels were quantified using a Nano Glo HiBiT Lytic Degradation Assay following the manufacturer's protocol. Briefly, the Nano-Glo® HiBiT Lytic Reagent was prepared by diluting the LgBiT Protein reagent (1:100) and the Nano-Glo® HiBiT Lytic Substrate (1:50) into Nano-Glo® HiBiT Lytic Buffer at room temperature. The 384-well microplate were equilibrated to room temperature and 25 μL of Nano-Glo® HiBiT Lytic Reagent was added to each well. Samples were mixed by placing the 384-well microplate on an orbital shaker for 10 minutes. Luminescence was measured using an EnVision 2105 Multimode Plate Reader.

Data processing and analysis: Background signal (wells containing growth medium without cells) was subtracted from individual relative light units (RLU) values and the resulting values were normalized to the DMSO control well values. Dose responses were analyzed with Genedata Screener® using a bell-shape curve fit and the concentrations corresponding to the half-maximal effect of the first Hill model were used as DC₅₀ values.

Results of the Nano Glo HiBiT Lytic Degradation Assay are presented in Table 6.

TABLE 6
HiBit assay results.

| Compound No. | MCF7 ESR1 HiBiT - (DC₅₀, μM) |
|---|---|
| I-1 | 0.003 |
| I-2 | 10 |
| I-3 | 0.00027 |
| I-4 | 0.05944 |
| I-5 | 10 |
| I-6 | 0.00224 |
| I-7 | 10 |
| I-8 | 10 |
| I-9 | 0.011 |
| I-10 | 0.016 |
| I-11 | 10 |
| I-13 | 0.013 |
| I-24 | 10 |
| I-25 | 0.00002 |
| I-28 | 10 |
| I-29 | 0.00014 |
| I-30 | 0.002 |
| I-31 | 10 |
| I-32 | 0.00045 |
| I-33 | 0.173 |
| I-34 | 0.00049 |
| I-35 | 0.0003 |
| I-36 | 0.08 |
| I-39 | 0.469 |
| I-40 | 0.37 |
| I-41 | 0.37 |
| I-42 | 10 |
| I-44 | 0.007 |
| I-45 | 0.003 |
| I-46 | 10 |
| I-47 | 0.079 |
| I-48 | 10 |
| I-49 | 10 |
| I-50 | 10 |
| I-51 | 0.00045 |
| I-52 | 0.009 |
| I-53 | 0.00092 |
| I-54 | 0.742 |
| I-55 | 0.00041 |
| I-56 | 0.007746 |
| I-57 | 0.007 |
| I-58 | 0.000048 |
| I-59 | 0.906 |
| I-60 | 0.001 |
| I-61 | 10 |
| I-62 | 0.002 |
| I-76 | 0.00021 |
| I-77 | 0.0002 |
| I-83 | 0.001 |
| I-85 | 0.00064 |
| I-98 | 10 |
| I-99 | 0.00067 |
| I-100 | 0.00033 |
| I-101 | 0.008 |
| I-102 | 10 |
| I-103 | 10 |
| I-104 | 0.00085 |
| I-105 | 0.04583 |
| I-106 | 1.03 |
| I-107 | 10 |
| I-108 | 0.01732 |
| I-109 | 1.396 |
| I-110 | 0.000155 |
| I-111 | 0.332 |
| I-112 | 0.00014 |
| I-114 | 10 |
| I-115 | 0.00063 |
| I-116 | 10 |
| I-117 | 10 |
| I-118 | 0.6 |
| I-119 | 0.006 |
| I-120 | 10 |
| I-121 | 0.003 |
| I-122 | 10 |
| I-123 | 0.00031 |
| I-124 | 0.001 |
| I-125 | 3.3 |
| I-126 | 0.00007 |
| I-127 | 10 |
| I-128 | 0.0001 |
| I-129 | 0.005 |
| I-130 | 10 |
| I-131 | 0.00003 |
| I-132 | 0.00004907 |
| I-133 | 10 |
| I-134 | 10 |
| I-135 | 0.0011 |
| I-136 | 10 |
| I-137 | 10 |
| I-138 | 10 |
| I-139 | 0.00076 |
| I-140 | 10 |
| I-141 | 0.002 |
| I-142 | 10 |
| I-143 | 10 |
| I-144 | 0.00037 |
| I-145 | 10 |
| I-146 | 0.003 |
| I-147 | 0.00042 |
| I-148 | 0.424 |
| I-149 | 0.00082 |
| I-150 | 10 |
| I-151 | 0.01104 |
| I-152 | 0.00143 |
| I-153 | 0.013 |
| I-154 | 3.3 |
| I-155 | 0.001 |
| I-156 | 0.0004 |
| I-157 | 0.00086 |
| I-158 | 1.1 |
| I-159 | 0.01 |
| I-160 | 10 |
| I-161 | 0.008 |
| I-162 | 0.003 |
| I-163 | 0.008 |
| I-174 | 0.018 |
| I-175 | 0.00077 |
| I-176 | 3.3 |
| I-177 | 10 |
| I-178 | 3.3 |
| I-179 | 0.034 |
| I-180 | 0.001305 |
| I-182 | 0.002 |
| I-183 | 10 |
| I-184 | 0.18171 |
| I-185 | 0.737 |
| I-188 | 0.00123 |
| I-189 | 0.002 |
| I-190 | 0.0004 |
| I-191 | 0.00109 |
| I-192 | 0.00062 |
| I-194 | 0.00041 |
| I-196 | 0.002 |
| I-197 | 0.00049 |
| I-198 | 0.00093 |
| I-199 | 0.00033 |
| I-202 | 0.0003 |
| I-203 | 0.00023 |
| I-204 | 0.002 |
| I-205 | 0.00079 |
| I-219 | 10 |
| I-220 | 0.014 |
| I-221 | 10 |
| I-222 | 0.00032 |
| I-223 | 0.01 |
| I-224 | 0.00048 |
| I-226 | 0.059 |
| I-227 | 0.004 |
| I-228 | 0.015 |
| I-229 | 10 |
| I-230 | 0.001 |
| I-231 | 10 |
| I-232 | 0.00092 |
| I-233 | 0.031 |
| I-234 | 0.959 |
| I-235 | 10 |
| I-236 | 0.072 |
| I-237 | 10 |
| I-238 | 0.004 |
| I-239 | 1.992 |
| I-240 | 0.003 |
| I-241 | 0.37 |
| I-242 | 3.3 |
| I-243 | 0.003 |
| I-244 | 0.004 |
| I-245 | 0.00023 |
| I-246 | 0.00026 |
| I-247 | 0.011 |
| I-248 | 0.021 |
| I-249 | 0.00061 |
| I-250 | 0.064 |
| I-251 | 0.044 |
| I-252 | 0.022 |
| I-253 | 0.00045 |
| I-254 | 0.041 |
| I-255 | 0.00087 |
| I-256 | 0.018 |
| I-257 | 3.3 |
| I-258 | 0.001 |
| I-259 | 0.098 |
| I-260 | 0.00075 |
| I-261 | 0.019 |
| I-262 | 0.002 |
| I-263 | 3.3 |
| I-264 | 0.00043 |
| I-265 | 0.003 |
| I-266 | 0.017 |
| I-267 | 0.004 |
| I-268 | 10 |
| I-269 | 0.006 |
| I-270 | 10 |
| I-271 | 0.008 |
| I-272 | 0.004 |
| I-273 | 0.006 |
| I-274 | 0.001 |
| I-275 | 0.00025 |
| I-276 | 0.0005 |
| I-277 | 0.0003 |
| I-278 | 0.01213 |
| I-279 | 0.0165 |
| I-280 | 0.00076 |
| I-281 | 0.00051 |
| I-282 | 0.02449 |
| I-283 | 0.005 |
| I-284 | 10 |
| I-285 | 0.00027 |
| I-286 | 0.0911 |
| I-287 | 10 |
| I-288 | 10 |
| I-289 | 10 |
| I-290 | 0.00022 |
| I-291 | 0.0007 |
| I-292 | 0.00073 |
| I-293 | 0.00052 |
| I-294 | 0.00028 |
| I-295 | 0.00084 |
| I-296 | 0.001 |
| I-297 | 0.00074 |
| I-298 | 0.001 |
| I-299 | 0.001 |
| I-300 | 3.3 |
| I-301 | 0.001 |
| I-302 | 0.002 |
| I-304 | 10 |
| I-305 | 0.00032 |
| I-306 | 0.0006 |
| I-307 | 0.019 |
| I-308 | 0.00065 |
| I-309 | 0.00039 |
| I-310 | 0.00574 |
| I-312 | 0.00037 |
| I-313 | 0.015 |
| I-314 | 0.00033 |
| I-315 | 10 |
| I-316 | 0.0003 |
| I-317 | 0.003 |
| I-318 | 0.00033 |
| I-319 | 0.108 |
| I-320 | 0.000006 |
| I-321 | 0.00044 |
| I-322 | 0.00056 |
| I-323 | 0.033 |
| I-324 | 0.00056 |
| I-325 | 0.011 |
| I-326 | 0.00058 |
| I-327 | 10 |
| I-328 | 0.009 |
| I-329 | 0.00092 |
| I-330 | 0.035 |
| I-331 | 10 |
| I-332 | 10 |
| I-333 | 0.021 |
| I-334 | 0.028 |
| I-335 | 0.00036 |
| I-336 | 0.015 |
| I-337 | 10 |
| I-338 | 0.01 |
| I-339 | 0.00031 |
| I-340 | 0.0005 |
| I-341 | 10 |
| I-342 | 0.00074 |
| I-343 | 0.00046 |
| I-344 | 0.084 |
| I-345 | 0.005 |
| I-346 | 10 |
| I-347 | 0.00018 |
| I-348 | 10 |
| I-349 | 0.00039 |
| I-350 | 0.02 |
| I-351 | 0.0002 |
| I-352 | 0.017 |
| I-354 | 0.0003 |
| I-355 | 0.00027 |
| I-356 | 0.054 |
| I-357 | 0.00051 |
| I-358 | 0.006 |
| I-359 | 0.00016 |
| I-360 | 0.00038 |
| I-361 | 10 |
| I-362 | 0.001 |
| I-363 | 0.00017 |
| I-364 | 10 |
| I-365 | 0.00024 |
| I-366 | 10 |
| I-367 | 0.00024 |
| I-368 | 0.003 |
| I-369 | 0.00034 |
| I-370 | 3.3 |
| I-371 | 0.00017 |
| I-372 | 0.027 |
| I-373 | 0.00078 |
| I-374 | 0.008 |
| I-375 | 0.00089 |
| I-376 | 10 |
| I-377 | 0.00062 |
| I-378 | 10 |
| I-379 | 0.00037 |
| I-380 | 0.077 |
| I-381 | 0.00016 |
| I-382 | 0.002 |
| I-387 | 0.00016 |
| I-388 | 0.04 |
| I-389 | 0.00028 |
| I-390 | 0.022 |
| I-391 | 0.00042 |
| I-392 | 0.00013 |
| I-393 | 0.00036 |
| I-394 | 0.00039 |
| I-395 | 0.00028 |
| I-396 | 0.00099 |
| I-397 | 0.00061 |
| I-398 | 0.00083 |
| I-399 | 10 |
| I-400 | 0.007746 |
| I-405 | 1.1 |
| I-406 | 0.025 |
| I-407 | 0.00019 |
| I-408 | 10 |
| I-409 | 10 |
| I-410 | 10 |
| I-411 | 0.001 |
| I-412 | 0.332 |
| I-413 | 0.00027 |
| I-414 | 0.00037 |
| I-415 | 0.00002 |
| I-416 | 0.00044 |
| I-418 | 0.00033 |
| I-419 | 0.002 |
| I-420 | 0.002 |
| I-421 | 0.00077 |
| I-422 | 0.141 |
| I-423 | 10 |
| II-1 | 0.002 |
| II-10 | 0.005 |
| II-100 | 0.0894 |
| II-101 | 10 |
| II-102 | 0.00015 |
| II-103 | 10 |
| II-104 | 0.022 |
| II-105 | 10 |
| II-106 | 0.00282 |
| II-107 | 0.00391 |
| II-108 | 0.00045 |
| II-109 | 0.00105 |
| II-11 | 10 |
| II-110 | 0.00088 |
| II-12 | 10 |
| II-13 | 0.018 |
| II-14 | 10 |
| II-15 | 10 |
| II-16 | 0.002 |
| II-17 | 0.0000803 |
| II-18 | 10 |
| II-19 | 10 |
| II-2 | 0.001 |
| II-20 | 10 |
| II-21 | 0.173 |
| II-22 | 0.005 |
| II-23 | 10 |
| II-24 | 0.245 |
| II-25 | 0.00117 |
| II-26 | 10 |
| II-27 | 0.00082 |
| II-28 | 0.02 |
| II-29 | 0.00124 |
| II-3 | 0.006 |
| II-30 | 0.1 |
| II-31 | 0.0009 |
| II-32 | 10 |
| II-33 | 0.002 |
| II-34 | 0.04 |
| II-35 | 0.00034 |
| II-36 | 0.00093 |
| II-37 | 0.046 |
| II-38 | 0.001 |
| II-39 | 10 |
| II-4 | 1.145 |
| II-40 | 10 |
| II-41 | 0.002 |
| II-42 | 10 |
| II-43 | 0.002 |
| II-44 | 0.002 |
| II-47 | 0.002 |
| II-48 | 0.001 |
| II-49 | 0.006 |
| II-5 | 0.002 |
| II-51 | 10 |
| II-52 | 0.00015 |
| II-53 | 0.00022 |
| II-56 | 0.000006 |
| II-57 | 10 |
| II-58 | 10 |
| II-59 | 10 |
| II-6 | 10 |
| II-60 | 10 |
| II-61 | 0.00008 |
| II-62 | 10 |
| II-64 | 0.016 |
| II-65 | 0.004 |
| II-66 | 0.00033 |
| II-67 | 0.047 |
| II-68 | 0.0005 |
| II-69 | 0.00051 |
| II-7 | 10 |
| II-70 | 0.005 |
| II-71 | 10 |
| II-72 | 10 |
| II-73 | 0.361 |
| II-74 | 0.00013 |
| II-75 | 0.00073 |
| II-76 | 10 |
| II-77 | 10 |
| II-78 | 0.00137 |
| II-79 | 10 |
| II-8 | 10 |
| II-80 | 0.00068 |
| II-81 | 10 |
| II-82 | 0.00084 |
| II-83 | 0.424 |
| II-84 | 0.0008 |
| II-85 | 1.086 |
| II-86 | 0.00061 |
| II-88 | 0.002 |
| II-89 | 1.1 |
| II-9 | 0.021 |
| II-91 | 0.02 |
| II-92 | 0.006 |
| II-94 | 0.00064 |
| II-95 | 0.00083 |
| II-96 | 10 |
| II-97 | 0.00079 |
| II-98 | 10 |
| II-99 | 0.00281 |

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound of formula I-3′: 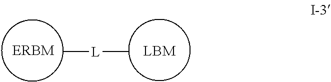
or a pharmaceutically acceptable salt thereof, wherein:
ERBM is selected from 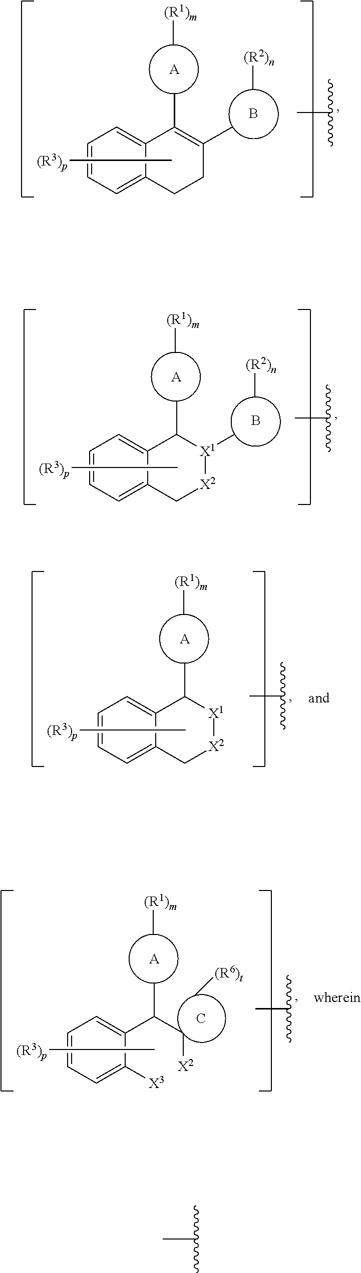
indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1}), or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3} R^{A4} and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 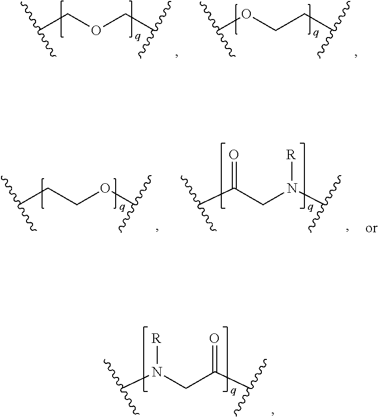
wherein:
each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from 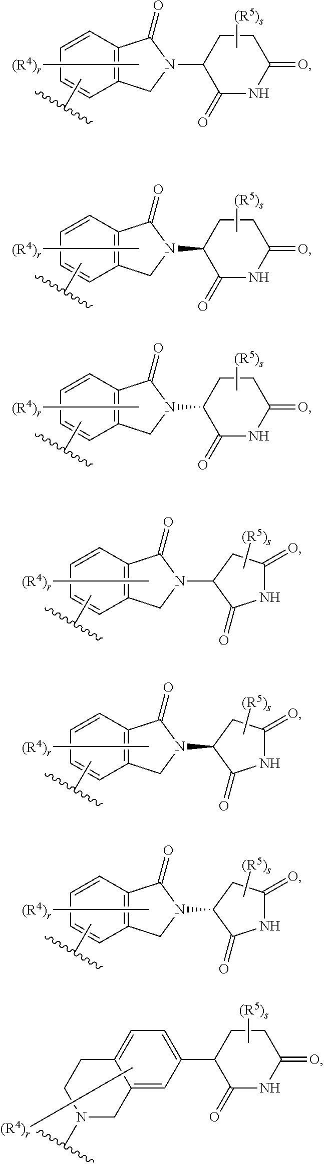 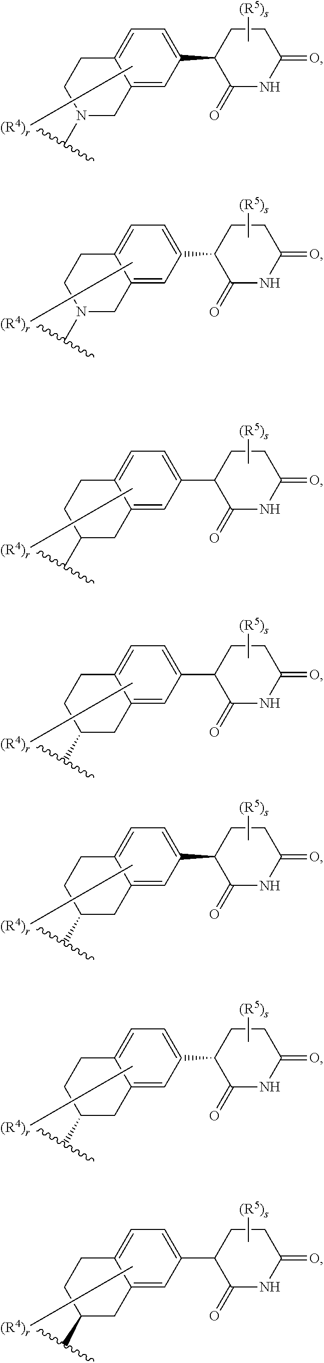 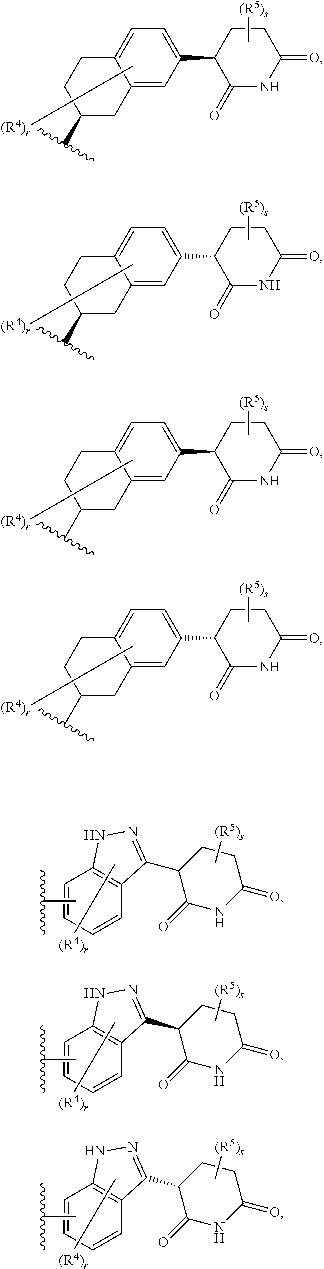 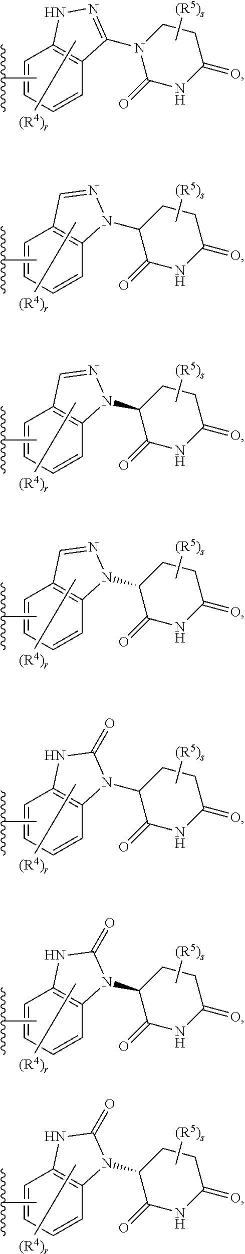 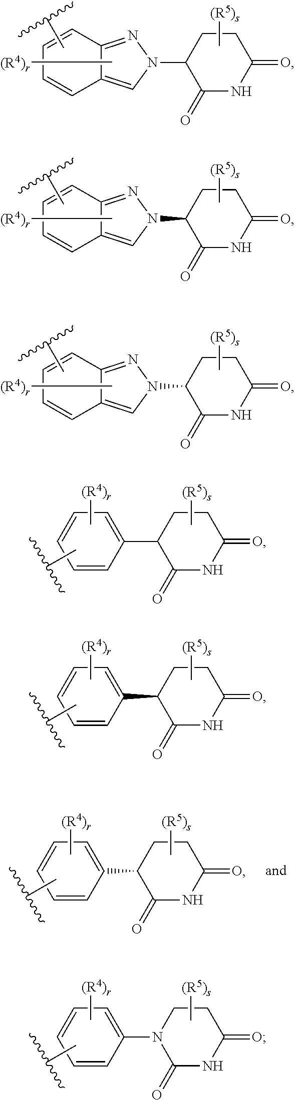
m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4.

2. A compound of formula I-3′: 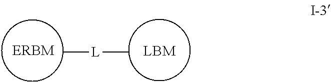
or a pharmaceutically acceptable salt thereof, wherein:
ERBM is selected from 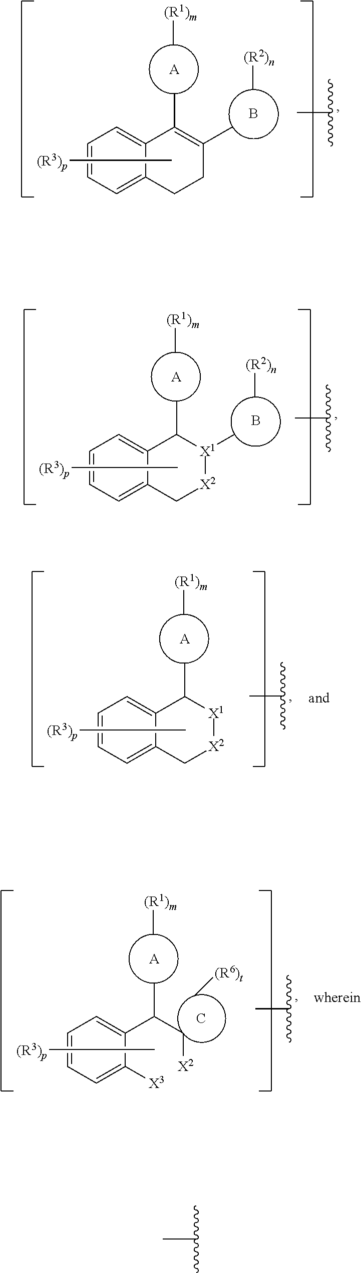
indicates the site of attachment of the -L-LBM moiety to a modifiable carbon, oxygen, nitrogen, or sulfur atom of the ERBM moiety;
X¹ is N, NH, CH, CH₂, CH(R^{A1}) or C(R^{A1})₂ or C(R^{A1}) as allowed by the other substituents;
X² is N(R^{A2}), O, CH₂, CH(R^{A3}), or C(R^{A3})₂;
X³ is N(R^{A4}), O, CH₂, CH(R^{A5}), or C(R^{A5})₂;
provided that X¹ and X² or X² and X³ are not both heteroatoms;
each instance of R¹, R², R⁴, R⁵, R⁶, R^{A1}, R^{A2}, R^{A3}, R^{A4}, and R^{A5} is independently R^{A} or R^{B}, and is substituted by 0-4 instances of R^{C};
each instance of R³ is independently R^{A} or R^{B}, and is substituted by 0-4 instances of R^{C}, or two R³ groups are optionally taken together to form a 5-8 membered partially unsaturated or aryl fused ring having 0-2 heteroatoms independently selected from nitrogen, oxygen, and sulfur;
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur;
Ring A and Ring B are each independently phenyl; naphthyl; tetrahydronaphthalenyl; dihydroindenyl; benzocyclobutenyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
Ring C is a spiro-fused 3-7 membered saturated or partially unsaturated monocyclic carbocyclic ring; a spiro-fused 5-12 membered saturated or partially unsaturated bicyclic carbocyclic ring; a spiro-fused 3-7 membered saturated or partially unsaturated monocyclic heterocyclic ring having 1-2 heteroatoms independently selected from nitrogen, oxygen, and sulfur; or a spiro-fused 7-12 membered saturated or partially unsaturated bicyclic heterocyclic ring having 1-4 heteroatoms independently selected from nitrogen, oxygen, and sulfur;
L is a covalent bond or a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 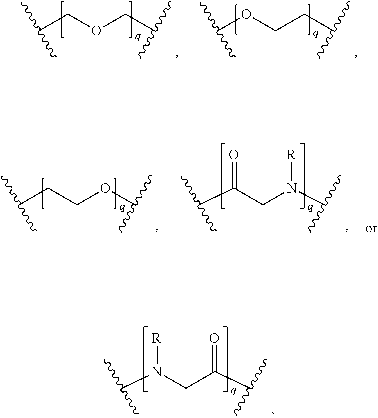
wherein:
each -Cy- is independently an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 6-10 membered bridged bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
LBM is selected from 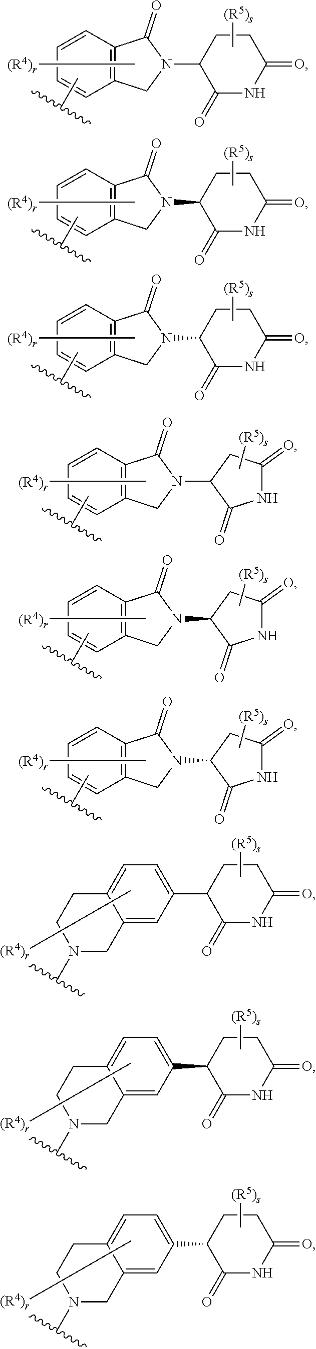 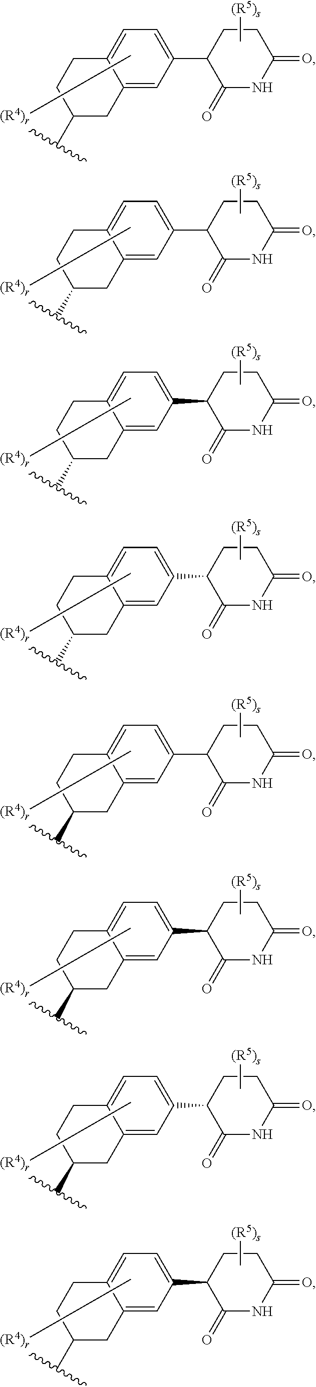 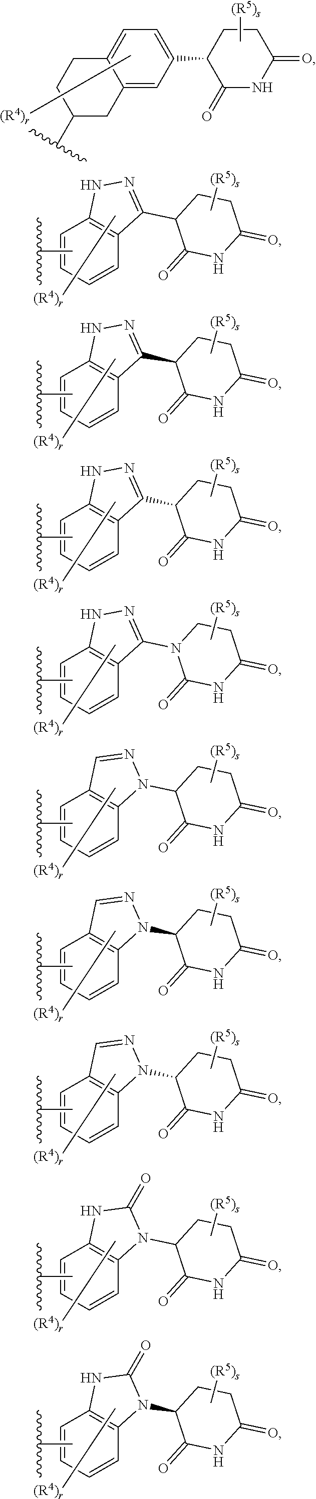 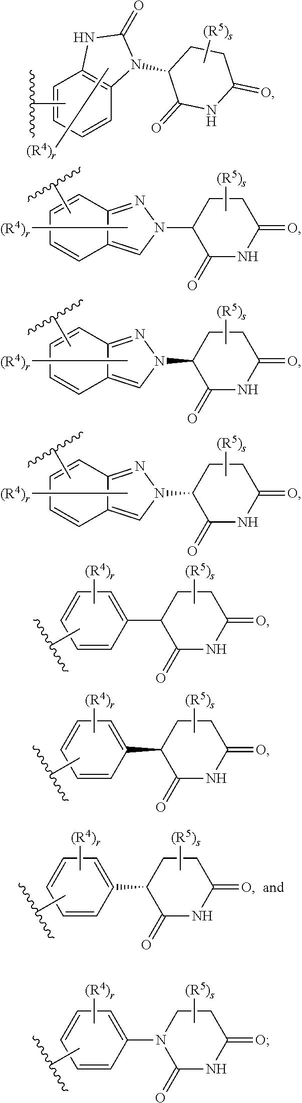
m is 0, 1, 2, 3, or 4;
n is 0, 1, 2, 3, or 4;
p is 0, 1, 2, 3, or 4;
each of q is independently 1, 2, 3, 4, 5, 6, 7, 8, 9, or 10;
r is 0, 1, 2, 3, or 4;
s is 0, 1, 2, 3, or 4; and
t is 0, 1, 2, 3, or 4,
provided that provided that when ERBM is 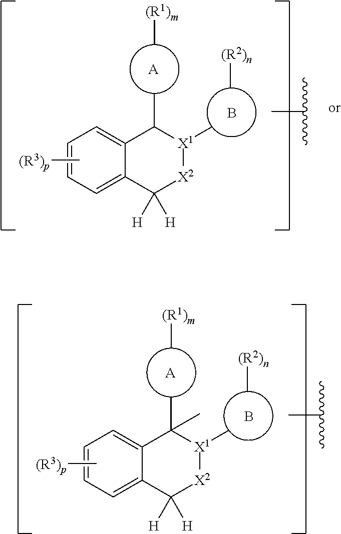
wherein X¹ is CH or N,
X² is CH₂;
Ring B is phenyl;
Ring A is phenyl or a 6 membered monocyclic heteroaryl ring having 1-3 nitrogen heteroatoms;
at least one R³ is —OH or —OMe;
p is 1, 2, or 3;
m is 0, 1 or 2;
R¹ is selected from —F and —Cl;
n is 0, 1 or 2; and
R² is selected from —OH, -Me, —OMe, —F, —Br, —CF; and —ⁱPr;
then LBM is not 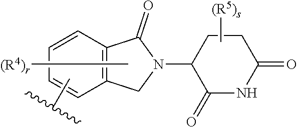
or a stereoisomer thereof.

3. The compound of claim 1 or 2, wherein ERBM is 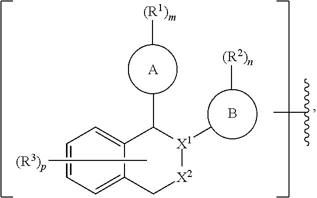
or a pharmaceutically acceptable salt thereof.

4. The compound of claim 1 or 2, wherein ERBM is 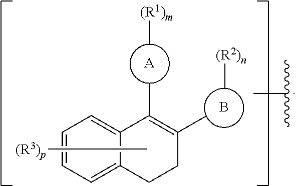
or a pharmaceutically acceptable salt thereof.

5. The compound of claim 1 or 2, wherein ERBM is 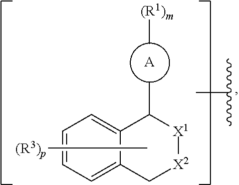
or a pharmaceutically acceptable salt thereof.

6. The compound of claim 1 or 2, wherein ERBM is 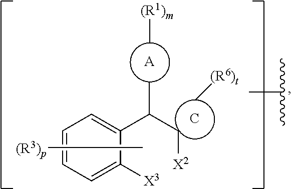
or a pharmaceutically acceptable salt thereof.

7. The compound of claim 1 or 2, wherein the compound is a compound of formula II-a, II-b, or II-c: 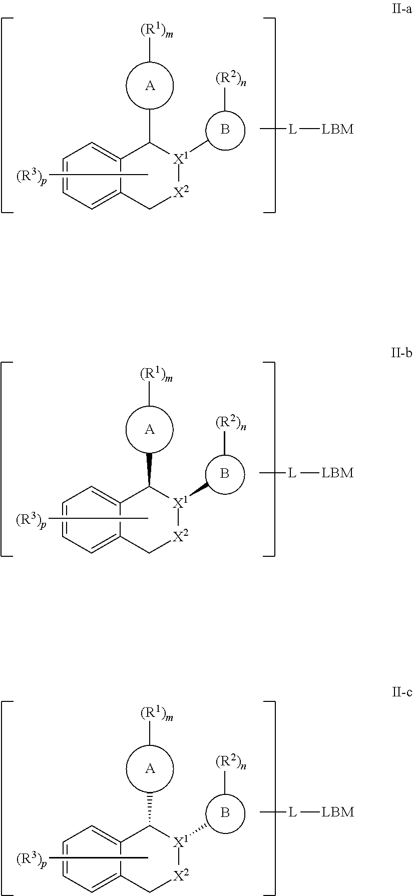
or a pharmaceutically acceptable salt thereof.

8. The compound of claim 1 or 2, wherein the compound is a compound of formula II-a-1, II-b-1, or II-c-1: 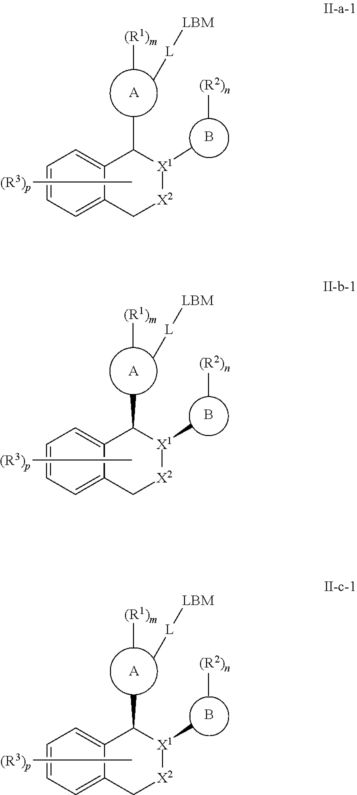
or a pharmaceutically acceptable salt thereof.

9. The compound of claim 1 or 2, wherein the compound is a compound of formula VIII-a, VIII-b, VIII-c, IX-a, IX-b or IX-c: 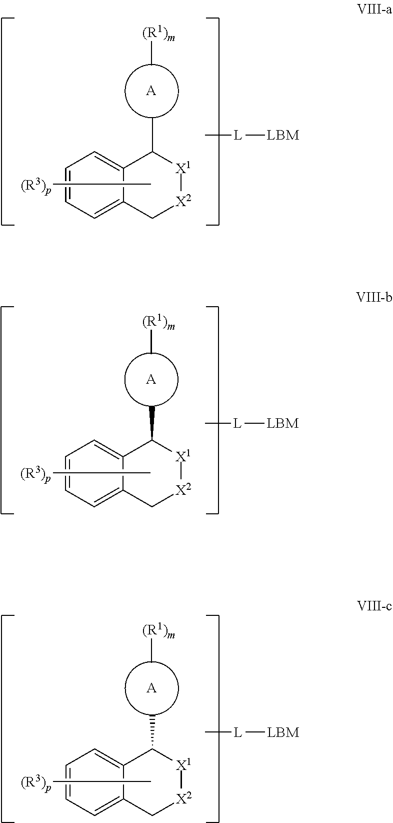 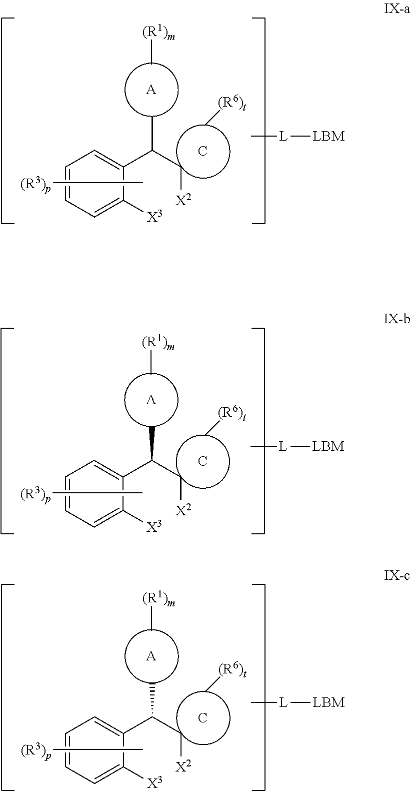
or a pharmaceutically acceptable salt thereof.

10. The compound of claim 1 or 2, wherein the compound is a compound of formula VIII-a-1, VIII-b-1, or VIII-c-1: 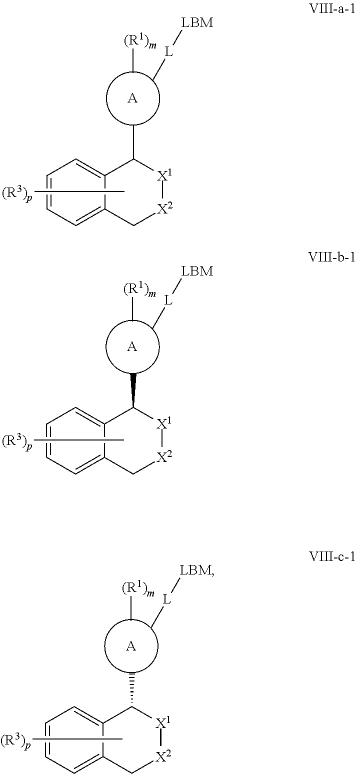
or a pharmaceutically acceptable salt thereof.

11. The compound of claim 1 or 2, wherein the compound is a compound of formula IX-a-1, IX-b-1 or IX-c-1 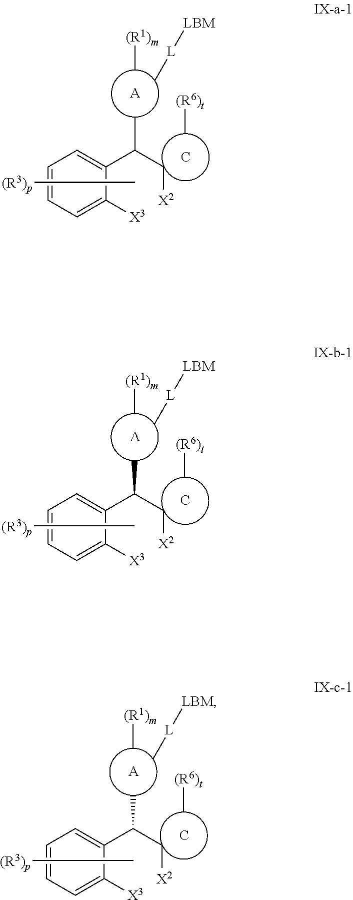
or a pharmaceutically acceptable salt thereof.

12. The compound of any one of claims 1-11, wherein X¹ is N or CH.

13. The compound of any one of claims 1-11, wherein X¹ is N.

14. The compound of any one of claims 1-11, wherein X¹ is CH.

15. The compound of any one of claims 1-11, wherein X¹ is NH or CH₂.

16. The compound of any one of claims 1-11, wherein X¹ is CH(R^{A1}).

17. The compound of any one of claims 1-11, wherein X¹ is C(R^{A1}).

18. The compound of any one of claims 1-11, wherein X¹ is C(R^{A1})₂.

19. The compound of any one of claims 1-11, wherein X² is O or CH₂, provided that X¹ and X² are not both simultaneously heteroatoms.

20. The compound of any one of claims 1-11, wherein X² is O.

21. The compound of any one of claims 1-11, wherein X² is CH₂.

22. The compound of any one of claims 1-11, wherein X³ is O, CH₂, CH(R^{A5}) or C(R^{A5}).

23. The compound of any one of claims 1-11, wherein X³ is O.

24. The compound of any one of claims 1-11, wherein X³ is CH₂.

25. The compound of any one of claims 1-11, wherein X³ is CH(R^{A5}).

26. The compound of any one of claims 1-11, wherein X³ is C(R^{A5}).

27. The compound of claim 1 or 2, wherein the compound is a compound of formula II-d-A, II-e-A, II-f-A, II-d-B, II-e-B, II-f-B, II-d-C, II-e-C, II-f-C, II-g, II-h, II-l, II-j-A, II-k-A, or II-l-A: 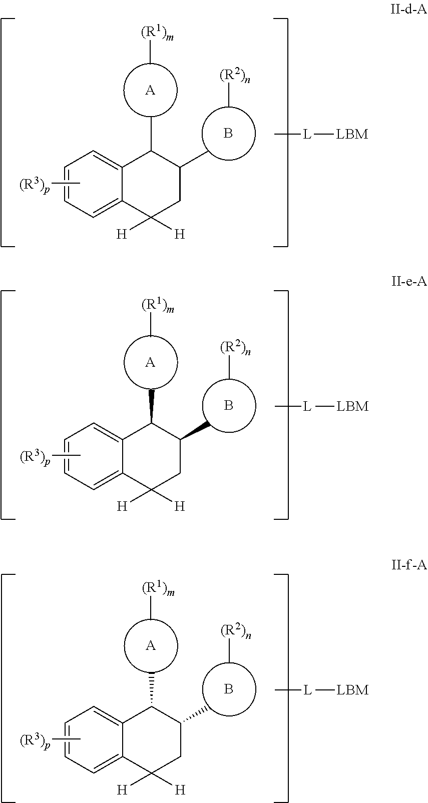 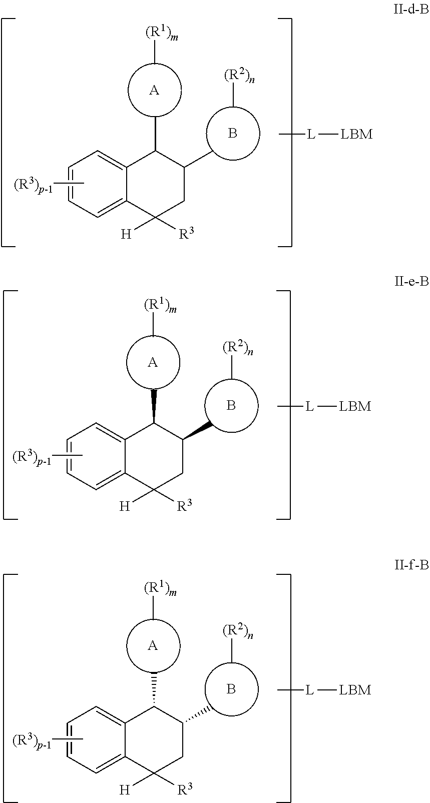 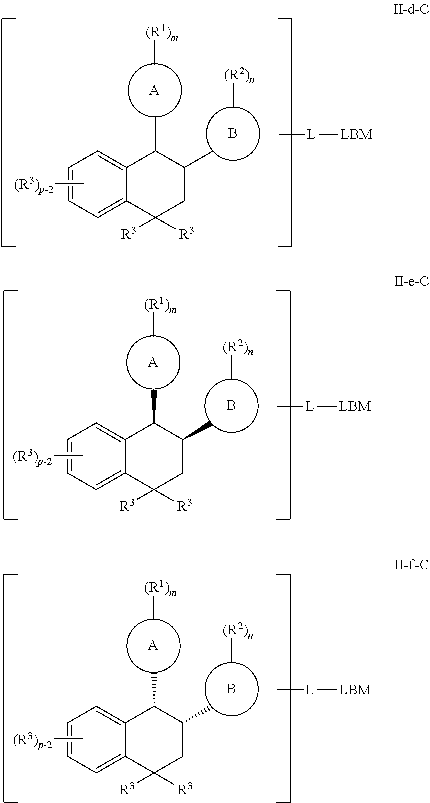 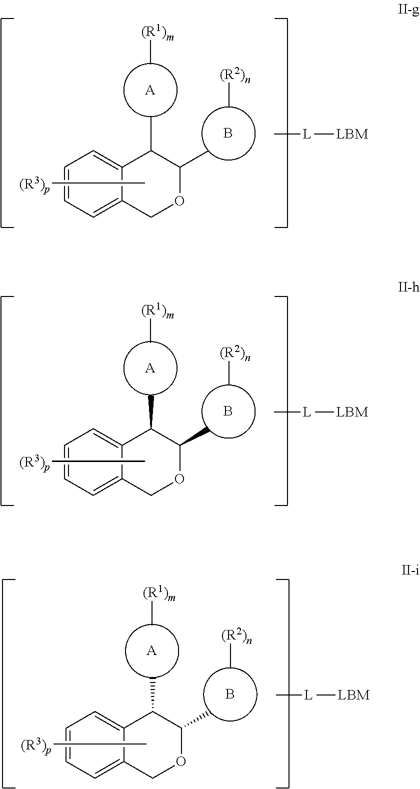 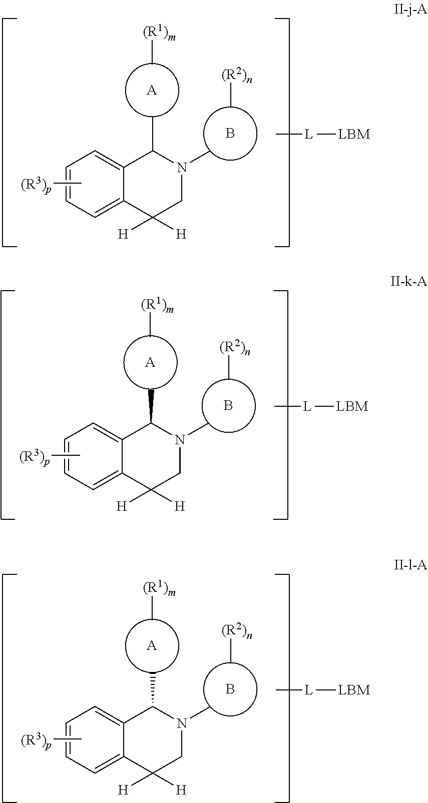
or a pharmaceutically acceptable salt thereof, provided that when the compound is of formula II-d-A, II-e-A, II-f-A, II-j-A, II-k-A and II-l-A and Ring B is phenyl, then Ring A is other than phenyl or a 6 membered monocyclic heteroaryl ring having 1-3 nitrogen heteroatoms.

28. The compound of claim 1 or 2, wherein the compound is a compound of one of formulas II-n, II-o, II-p, II-q, II-r, II-s, II-t, II-u or II-v: 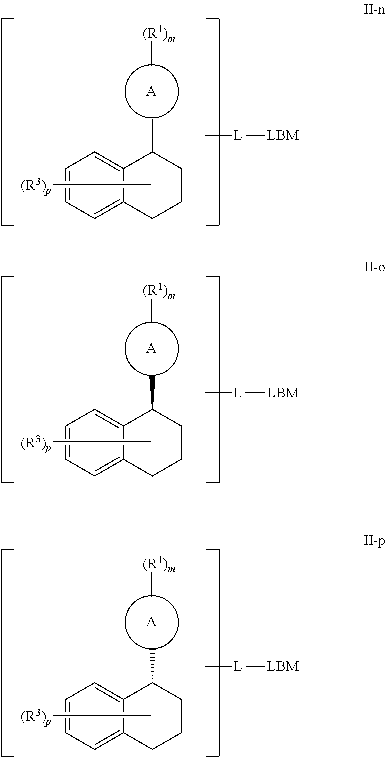 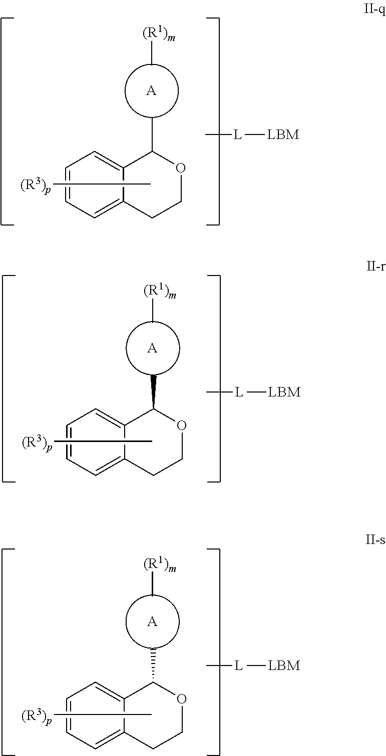 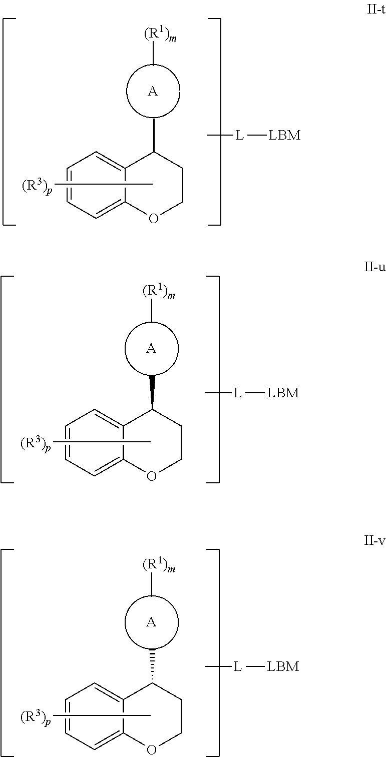
or a pharmaceutically acceptable salt thereof.

29. The compound of claim 1 or 2, wherein the compound is a compound of one of formulas II-w, II-x, II-y, II-z, II-aa, II-bb, II-cc, II-dd or II-ee: 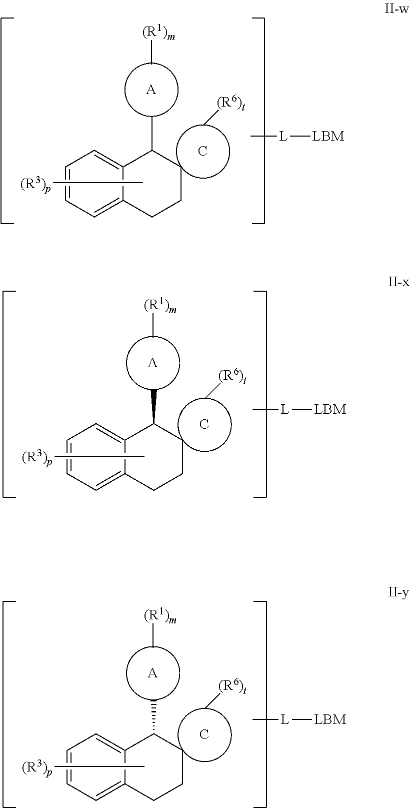 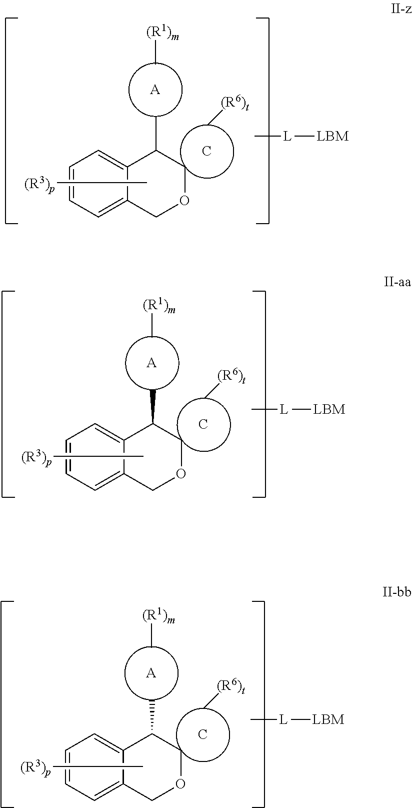 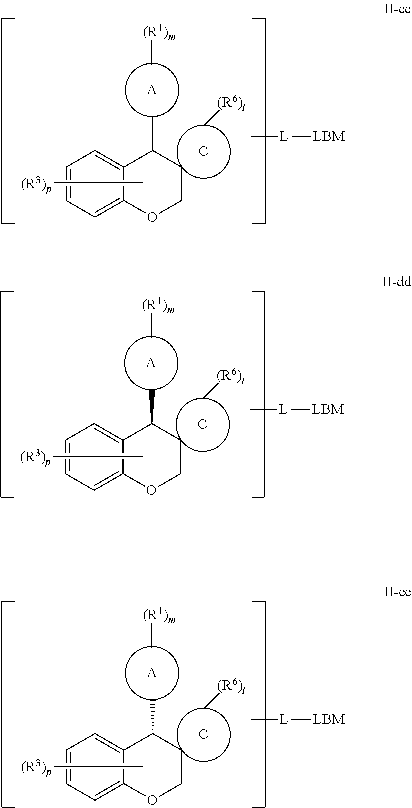
or a pharmaceutically acceptable salt thereof.

30. The compound of claim 1 or 2, wherein the compound is a compound of one of formulas II-d-1, II-e-1, II-f-1, II-g-1, II-h-1, II-i-1, II-j-1, II-k-1, or II-l-1: 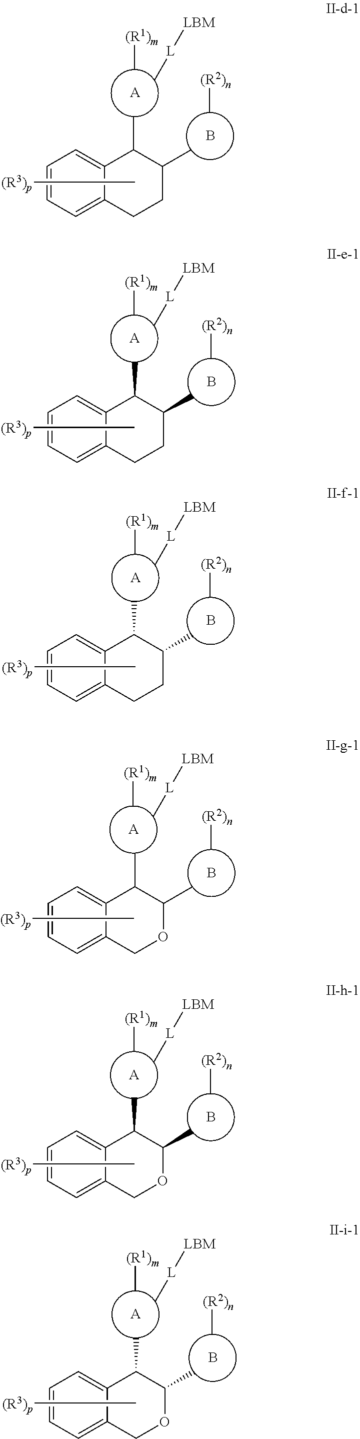 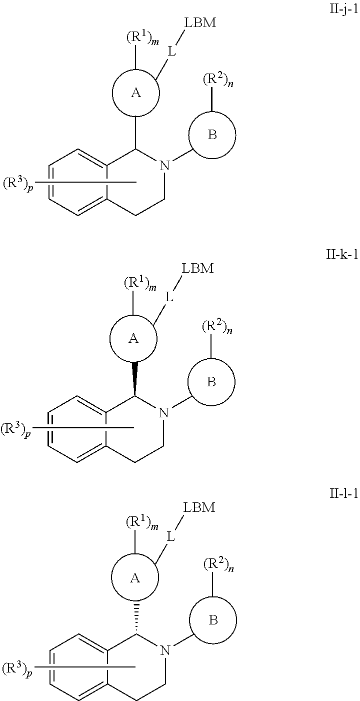
or a pharmaceutically acceptable salt thereof,
provided that when the compound is of formula II-d-1, II-e-1, II-f-1, II-j-1, II-k-1 and II-l-1 and Ring B is phenyl, then Ring A is other than phenyl or a 6 membered monocyclic heteroaryl ring having 1-3 nitrogen heteroatoms.

31. The compound of claim 1 or 2, wherein the compound is a compound of formula II-d-1-A, II-e-1-A, II-f-1-A, II-d-1-B, II-e-1-B, II-f-1-B, II-d-1-C, II-e-1-C, II-f-1-C, II-j-1-A, II-k-1-A, or II-l-1-A: 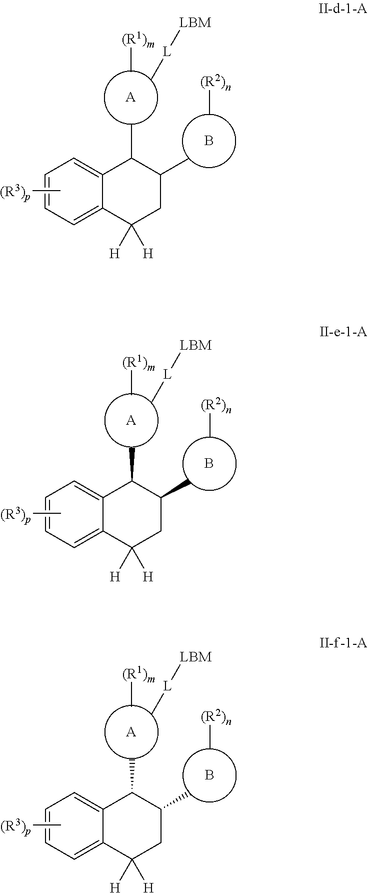 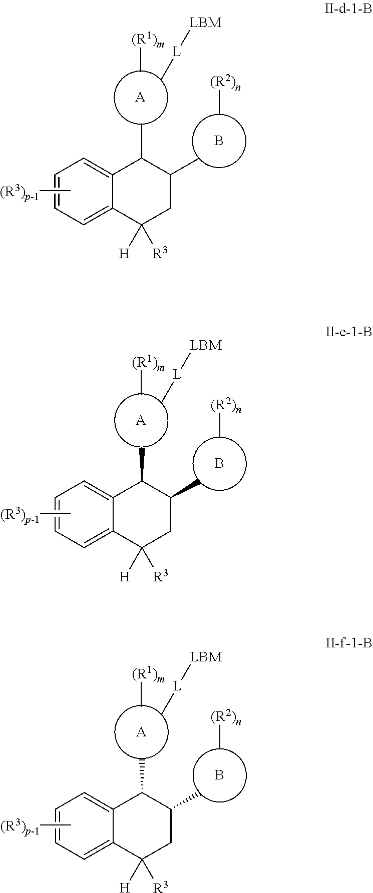 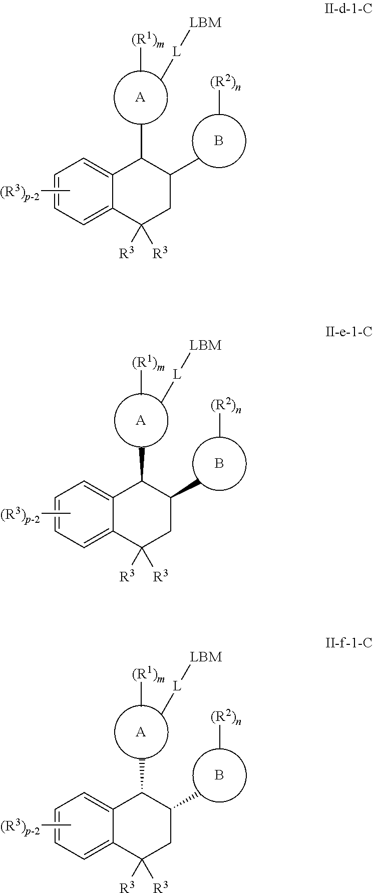 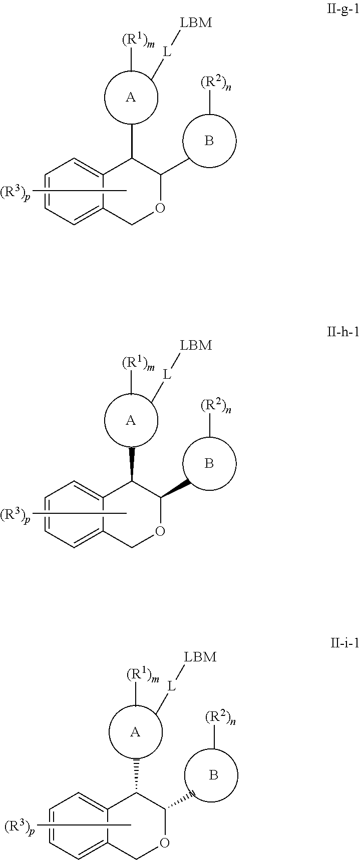 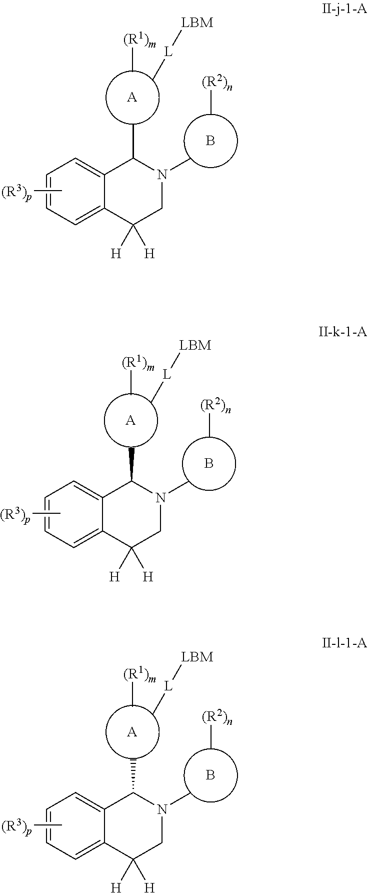
or a pharmaceutically acceptable salt thereof,
provided that when the compound is of formula II-d-1-A, II-e-1-A, II-f-1-A, II-j-1-A, II-k-1-A and II-l-1-A and Ring B is phenyl, then Ring A is other than phenyl or a 6 membered monocyclic heteroaryl ring having 1-3 nitrogen heteroatoms.

32. The compound of claim 1 or 2, wherein the compound is a compound of formula II-m-1: 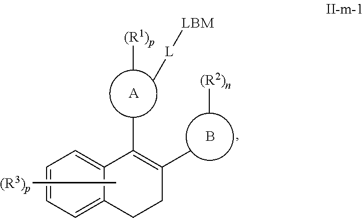
or a pharmaceutically acceptable salt thereof.

33. The compound of claim 1 or 2, wherein the compound is a compound of one of formulas II-n-1, II-o-1, II-p-1, II-q-1, II-r-1, II-s-1, II-t-1, II-u-1 or II-v-1: 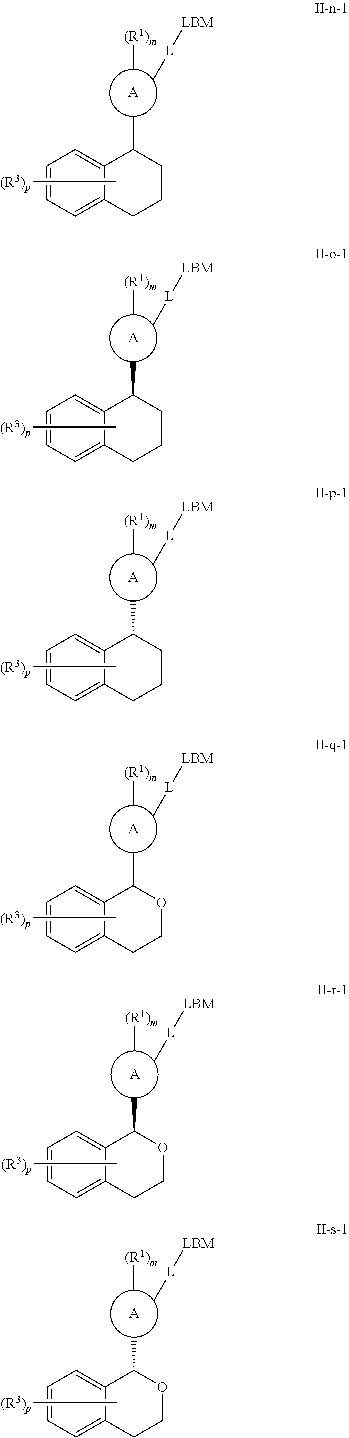 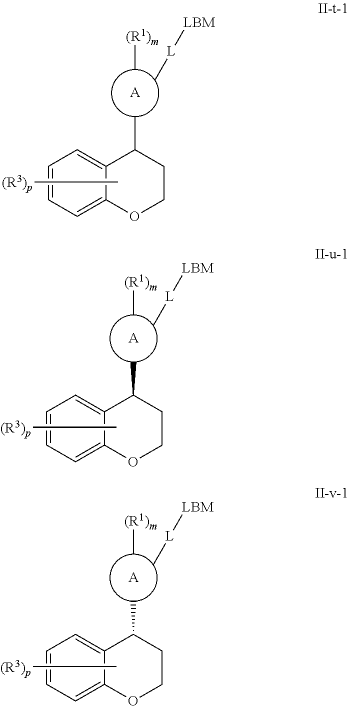
or a pharmaceutically acceptable salt thereof.

34. The compound of claim 1 or 2, wherein the compound is a compound of one of formulas II-w-1, II-x-1, II-y-1, II-z-1, II-aa-1, II-bb-1, II-cc-1, II-dd-1 or II-ee-1: 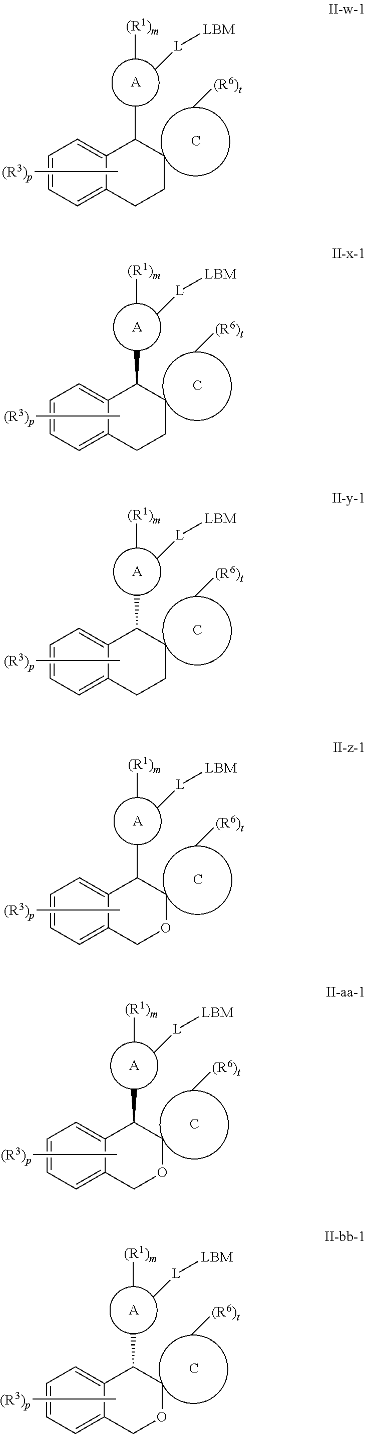 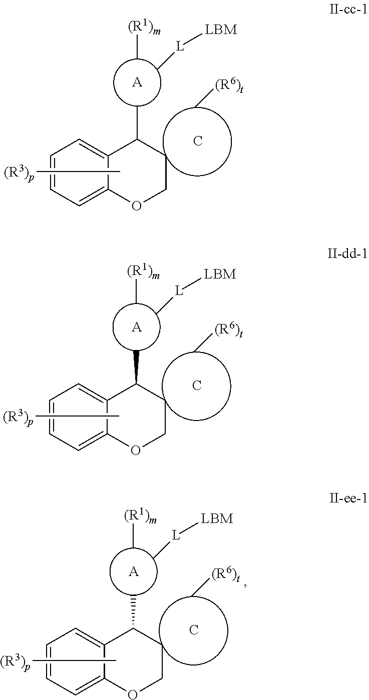
or a pharmaceutically acceptable salt thereof.

35. The compound of claim 1 or 2, wherein the compound is a compound of one of formulas II-ff-1, II-gg-1, II-hh-1, II-ii-1, II-jj-1, II-kk-1, II-ll-1, II-mm-1 or II-nn-1: 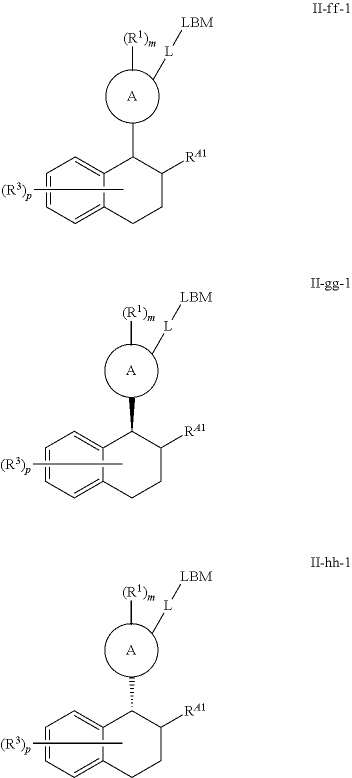 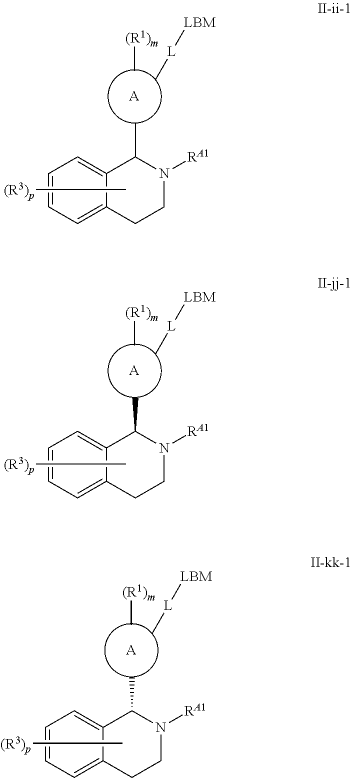 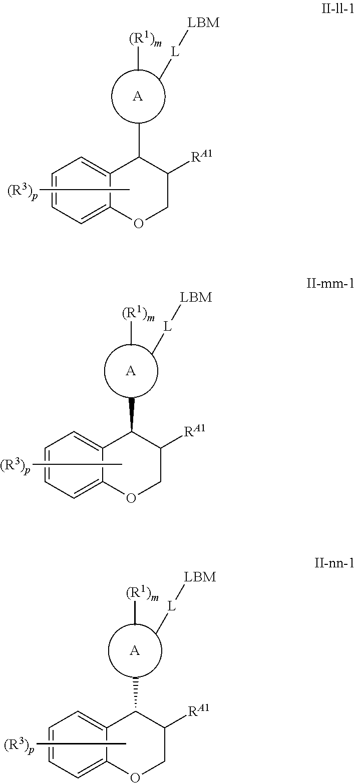
or a pharmaceutically acceptable salt thereof.

36. The compound of any one of claims 1-35, wherein Ring A together with its R¹ substituents is selected from 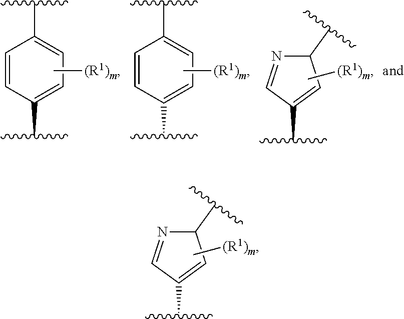
wherein the top attachment point connects to L and the bottom attachment point connects to the six-member ring of the ERBM moiety.

37. The compound of any one of claims 1-35, wherein Ring A together with its R¹ substituents is selected from 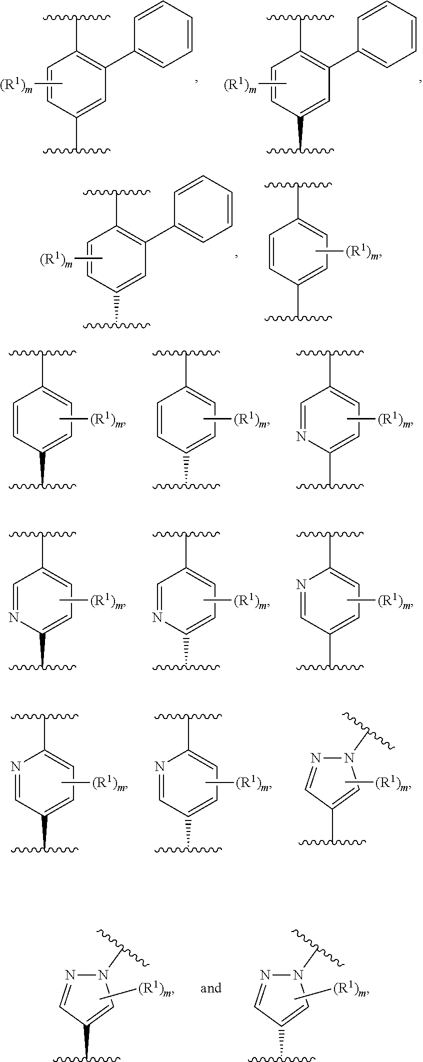
wherein the top attachment point connects to L and the bottom attachment point connects to the six-member ring of the ERBM moiety.

38. The compound of any one of claims 1-37, wherein Ring B together with its R² substituents is selected from:

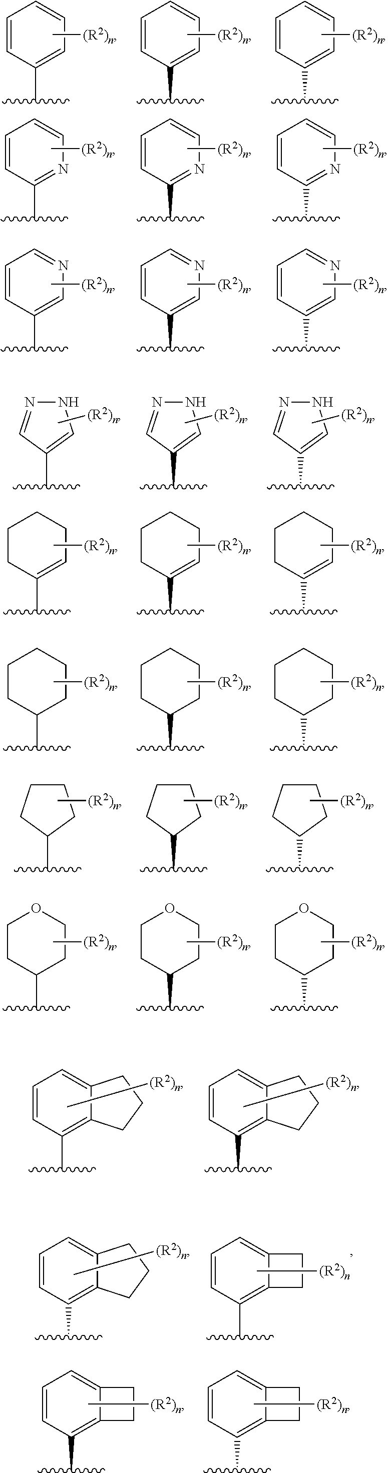

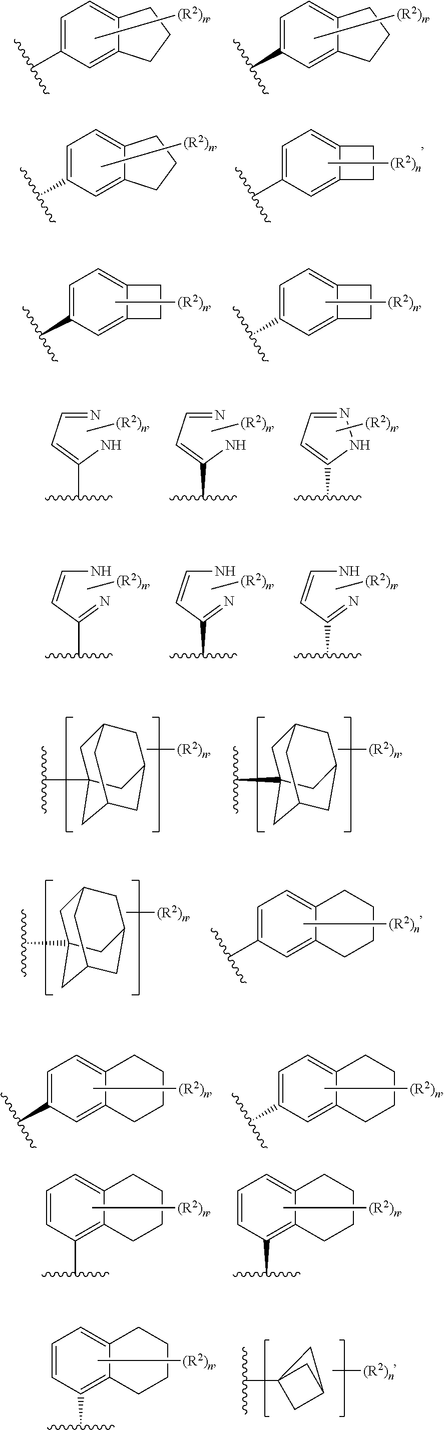

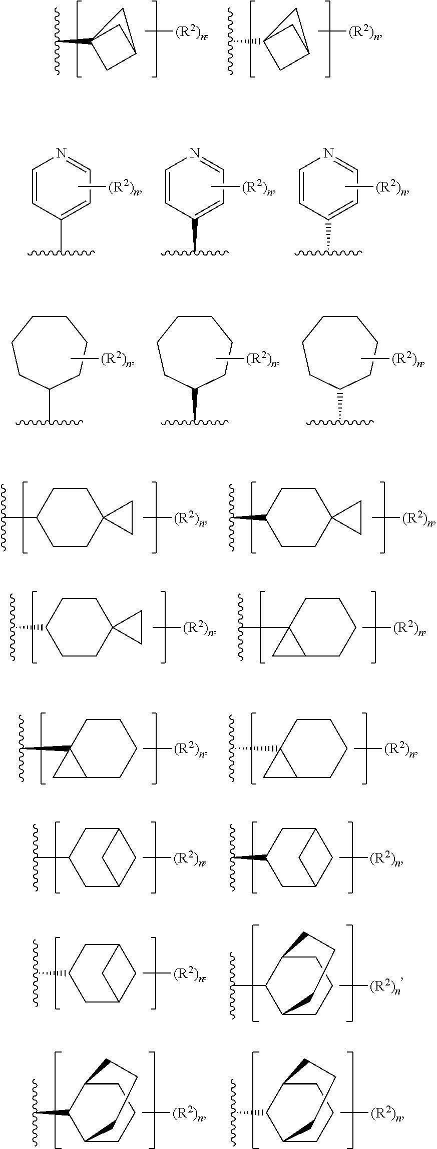

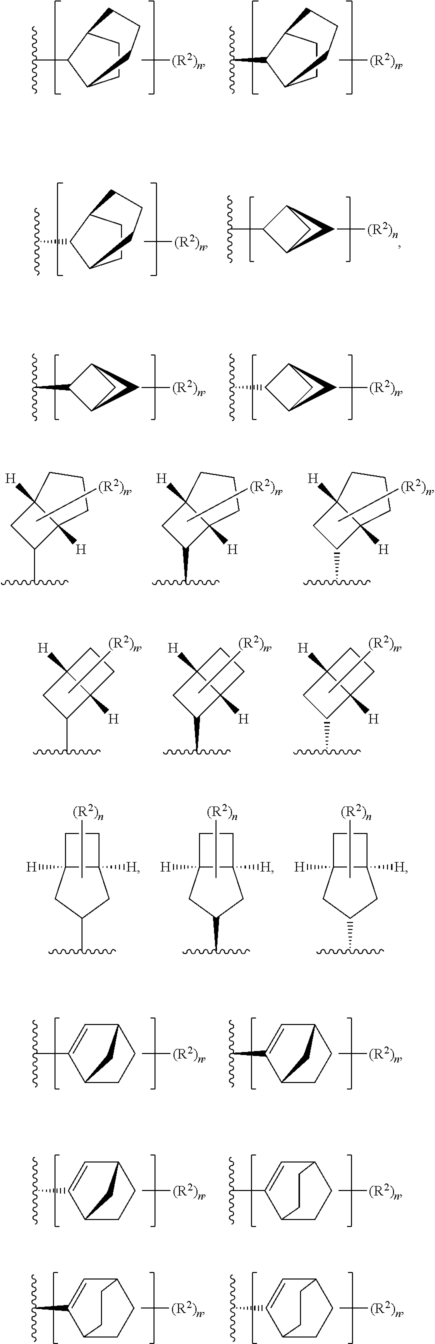

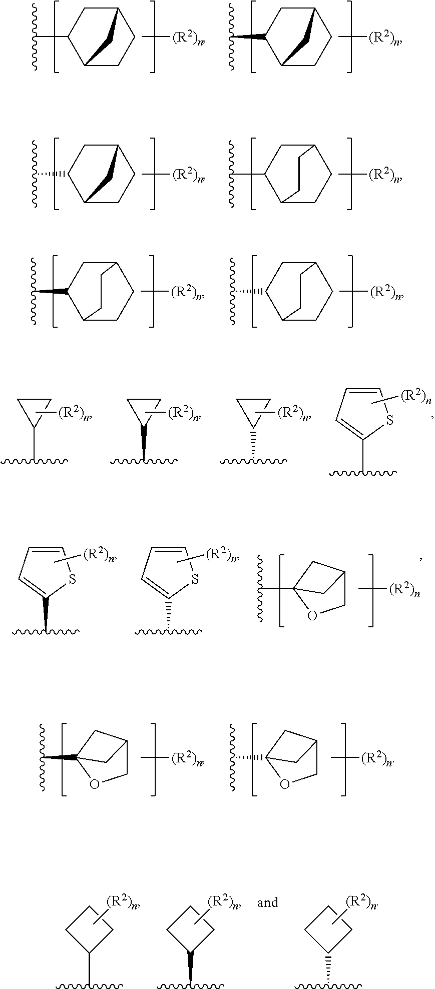

39. The compound of claim 1 or 2, wherein the compound is a compound of one of formulas III-a-A, III-b-A, III-c-A, III-a-B, III-b-B, III-c-B, III-a-C, III-b-C and III-c-C: 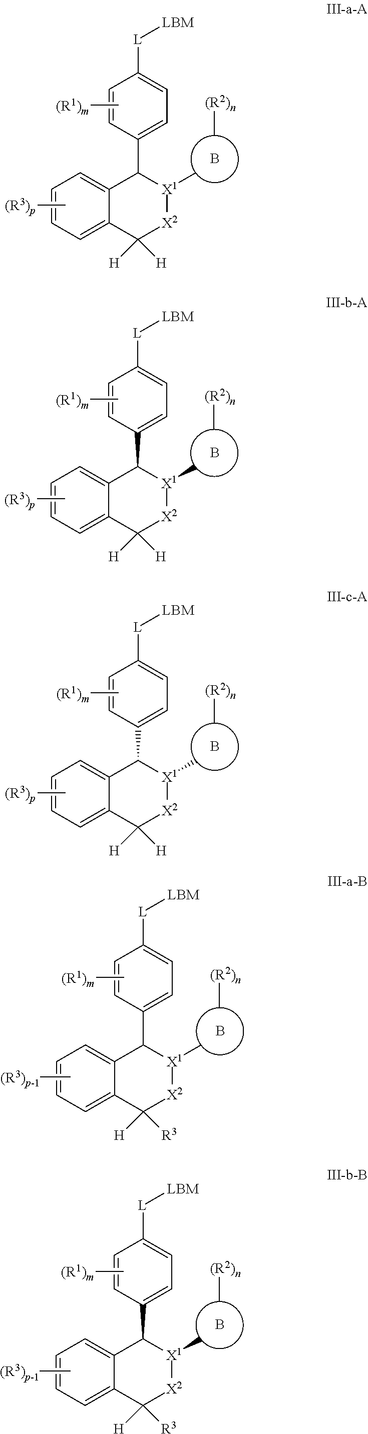 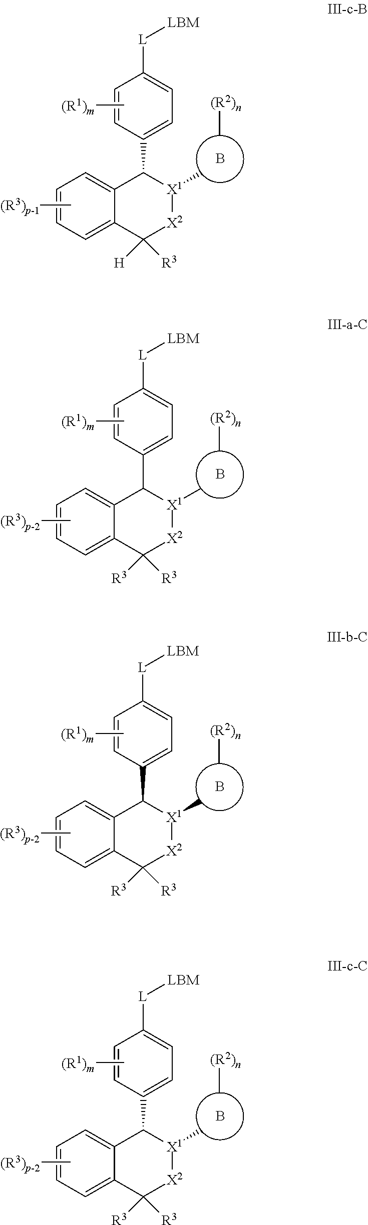
or a pharmaceutically acceptable salt thereof, provided that for formulae III-a-A, III-b-A and III-c-A when X¹ is CH or N and X² is CH₂, then Ring B is other than phenyl.

40. The compound of claim 1 or 2, wherein the compound is a compound of one of formulas III-d, III-e, III-f, III-g, III-h, III-i, III-j, III-k, or III-l: 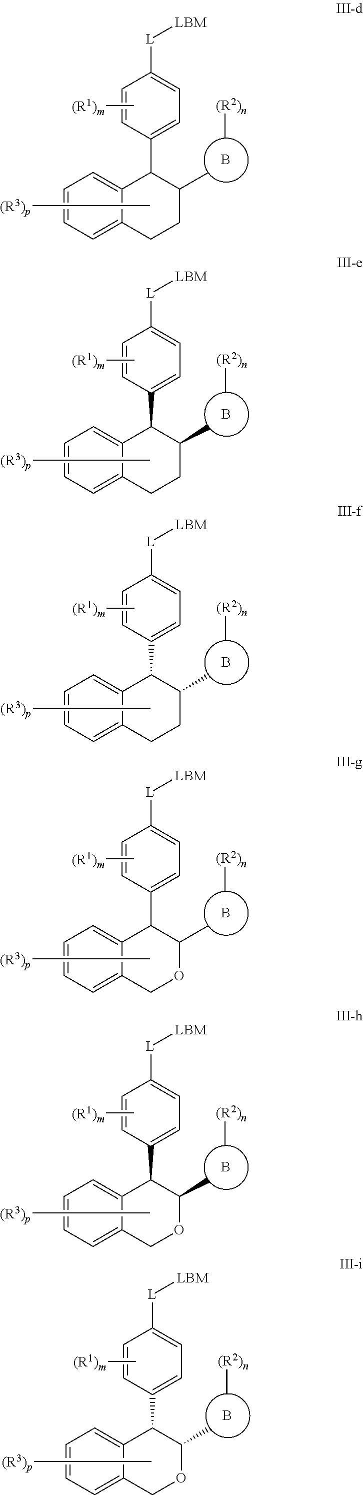 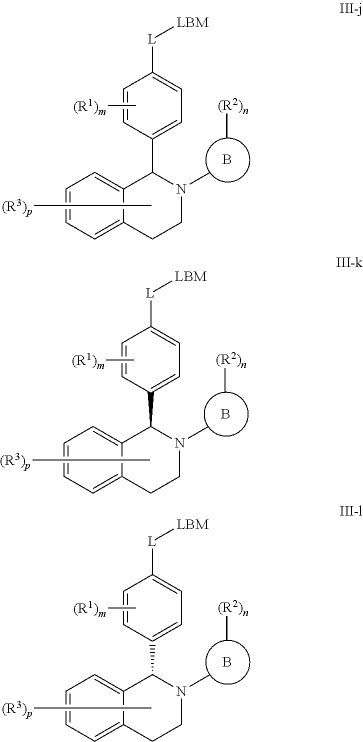
or a pharmaceutically acceptable salt thereof, provided that when the compound is of formula III-d, III-e, III-f, III-j, III-k or III-l, then Ring B is other than phenyl.

41. The compound of claim 1 or 2, wherein the compound is a compound of formula III-d-A, III-e-A, III-f-A, III-d-B, III-e-B, III-f-B, III-d-C, III-e-C, III-f-C, III-g, III-h, III-i, III-j-A, III-k-A, or III-l-A: 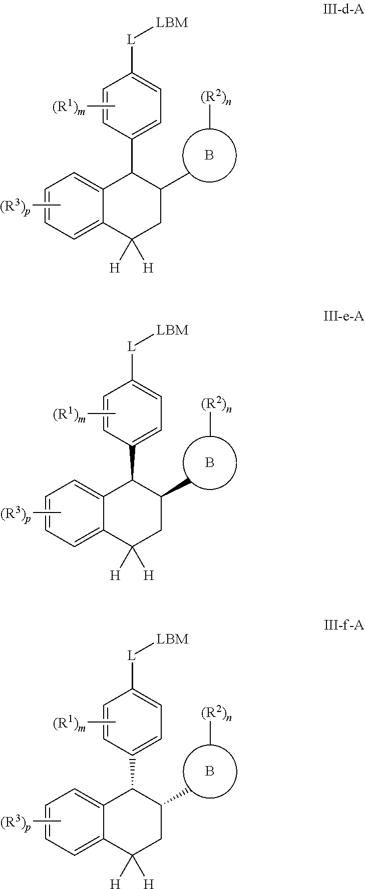 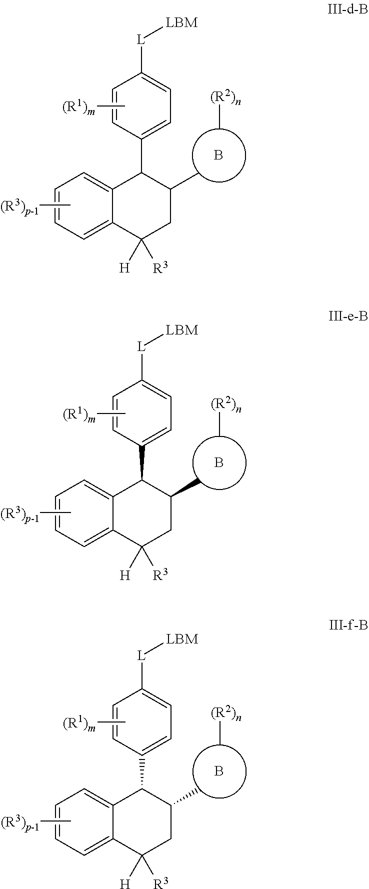 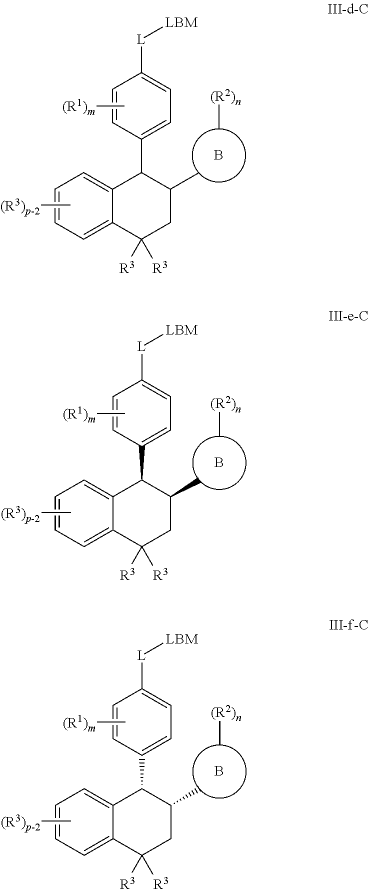 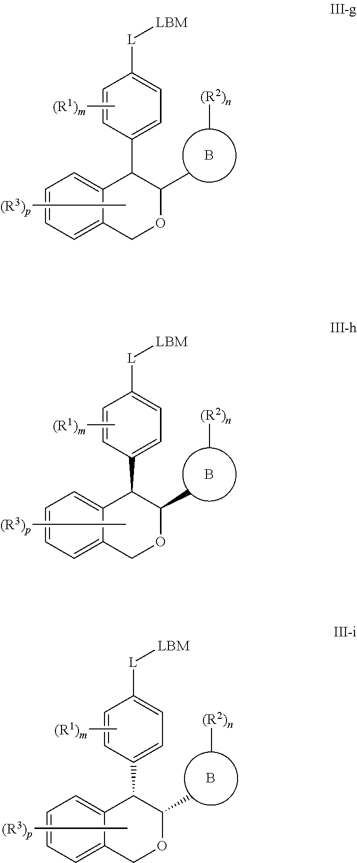 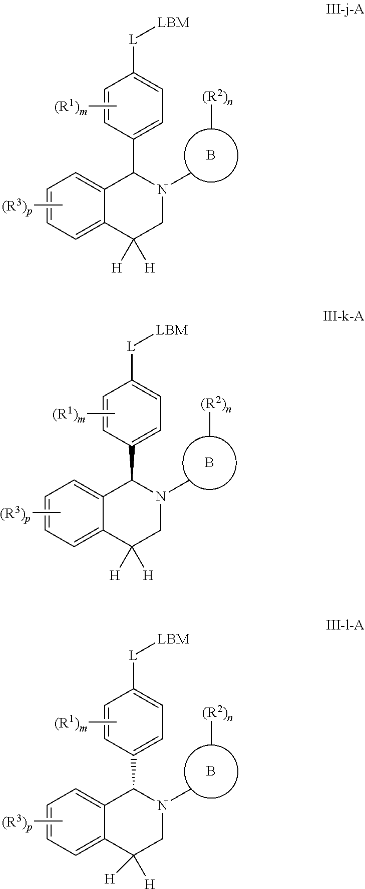
or a pharmaceutically acceptable salt thereof, provided that when the compound is of formula III-d-A, III-e-A, III-f-A, III-j-A, III-k-A, or III-l-A, then Ring B is other than phenyl.

42. The compound of claim 1 or 2, wherein the compound is a compound of one of formulas III-m, III-n, or III-o: 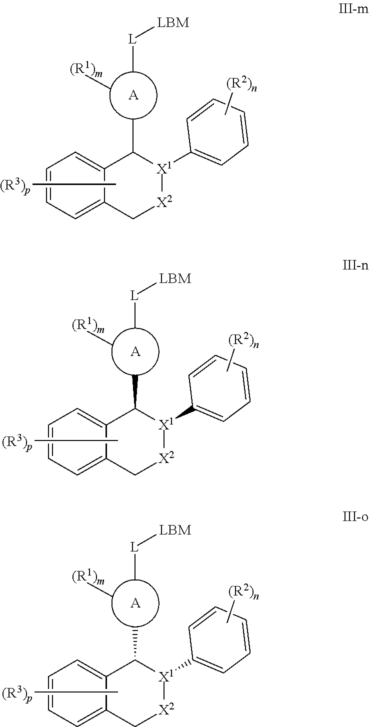
or a pharmaceutically acceptable salt thereof, provided that when X¹ is CH or N and X² is CH₂, then Ring A is other than phenyl or a 6 membered monocyclic heteroaryl ring having 1-3 nitrogen heteroatoms.

43. The compound of claim 1 or 2, wherein is a compound of one of formulas III-m-A, III-n-A, or III-o-A, III-m-B, III-n-B, III-o-B, III-m-C, III-n-C, or III-o-C: 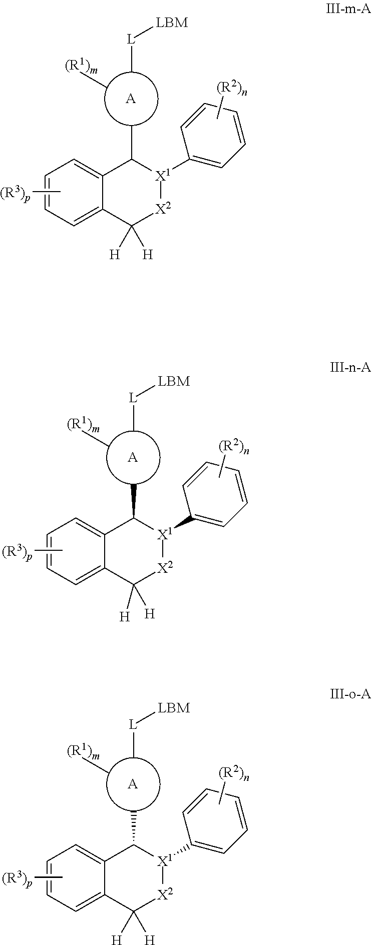 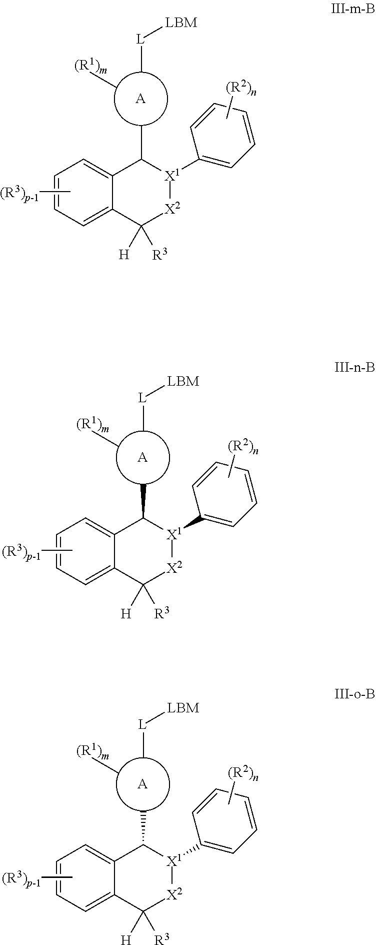 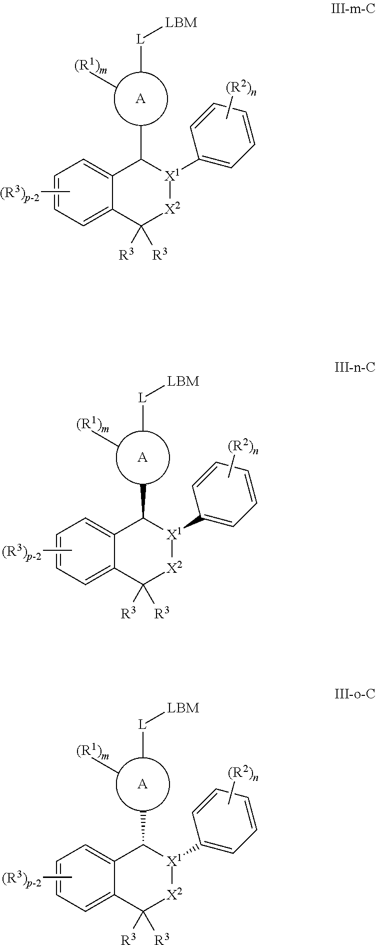
or a pharmaceutically acceptable salt thereof, provided that when the compound is of formulae III-m-A, III-n-A or III-o-A and X¹ is CH or N and X² is CH₂, then Ring A is other than phenyl or a 6 membered monocyclic heteroaryl ring having 1-3 nitrogen heteroatoms.

44. The compound of claim 1 or 2, wherein the compound is a compound of one of formulas III-p, III-q, III-r, III-s, III-t, III-u, III-v, III-w, or III-x: 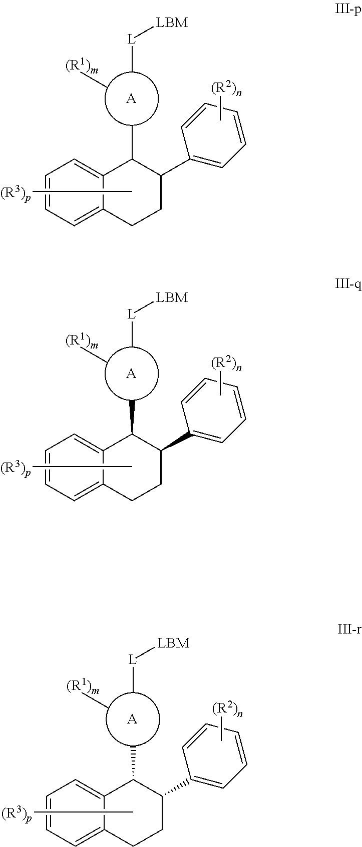 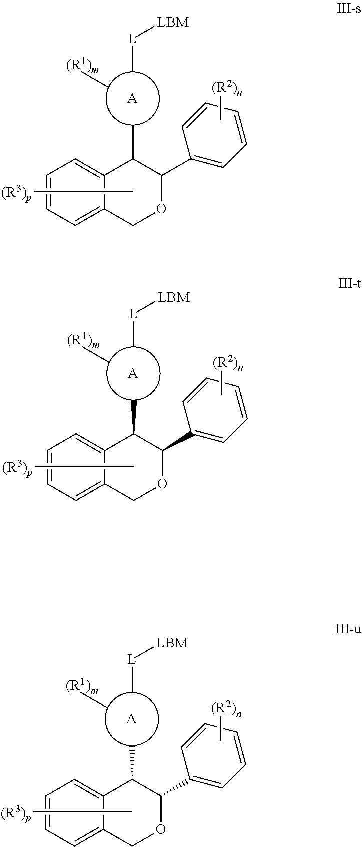 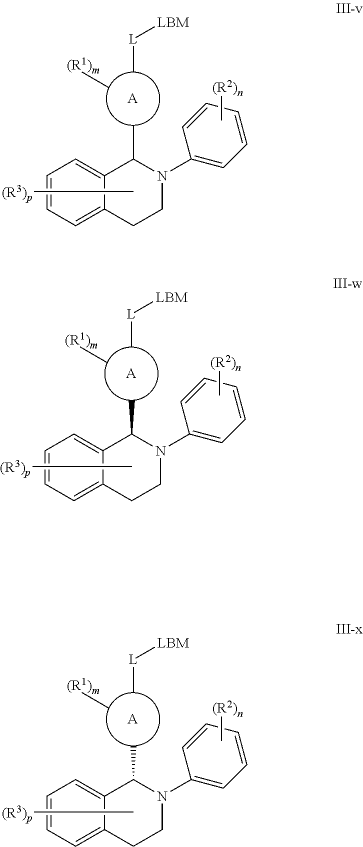
or a pharmaceutically acceptable salt thereof, provided that when the compound is of formulas III-p, III-q, III-r, III-v, III-w or III-x, then Ring A is other than phenyl or a 6 membered monocyclic heteroaryl ring having 1-3 nitrogen heteroatoms.

45. The compound of claim 1 or 2, wherein the compound is a compound of one of formulas III-p-A, III-q-A, III-r-A, III-p-B, III-q-B, III-r-B, III-p-C, III-q-C, III-r-C, III-s, III-t, III-u, III-v-A, III-w-A, or III-x-A: 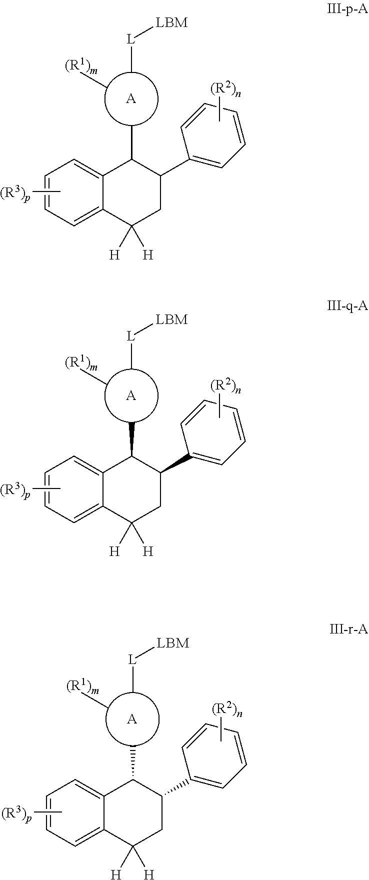 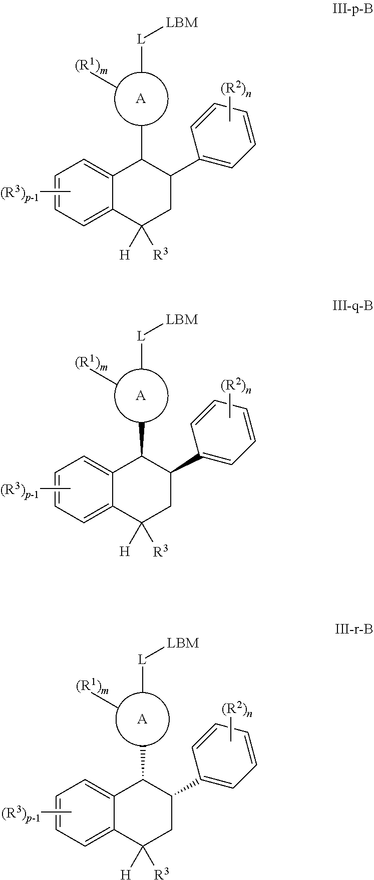 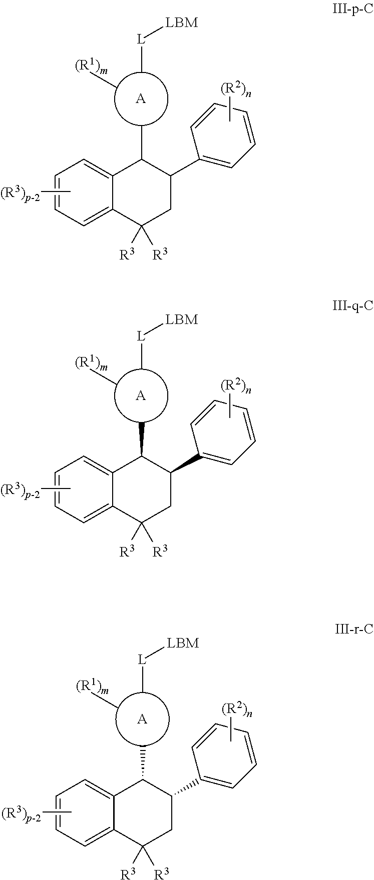 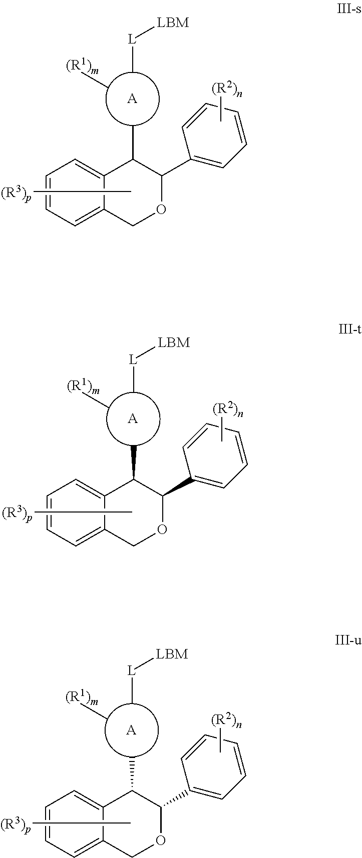 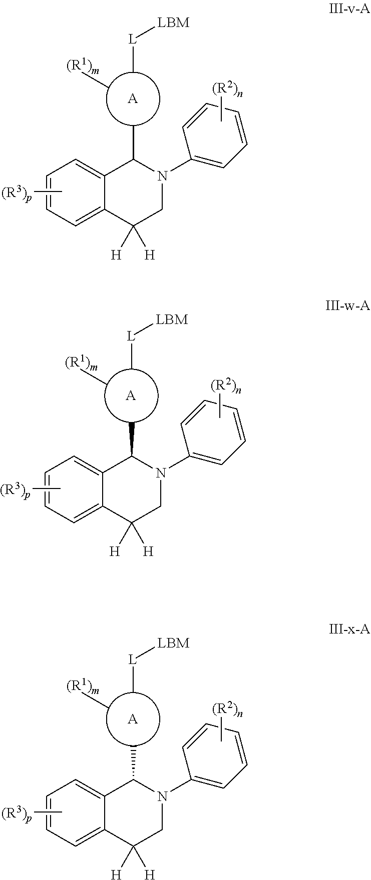
or a pharmaceutically acceptable salt thereof, provided that when the compound is of formulas III-p-A, III-q-A, III-r-A, III-v-A, III-w-A or III-x-A, then Ring A is other than phenyl or a 6 membered monocyclic heteroaryl ring having 1-3 nitrogen heteroatoms.

46. The compound of claim 1 or 2, wherein the compound is a compound of one of formulas IV-a, IV-b, or IV-c: 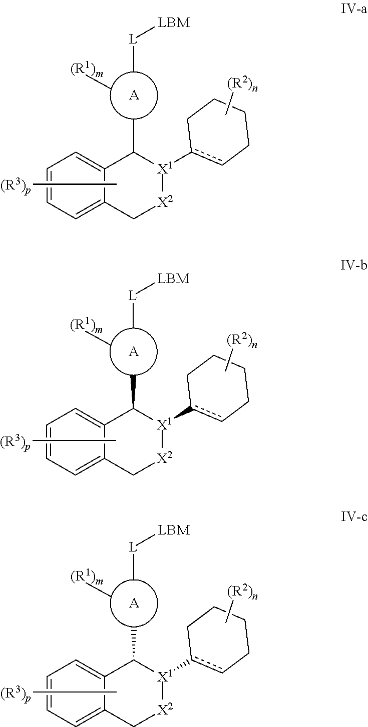
or a pharmaceutically acceptable salt thereof, wherein [symbol] represents a single or double bond.

47. The compound of claim 1 or 2, wherein the compound is a compound of one of formulas IV-d, IV-e, IV-f, IV-g, IV-h, IV-i, IV-j, IV-k, or IV-l: 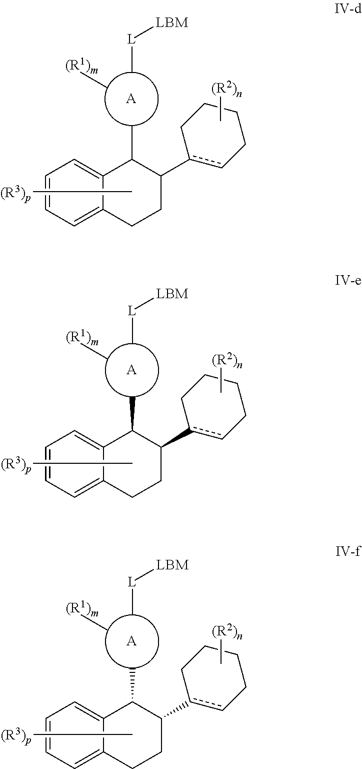 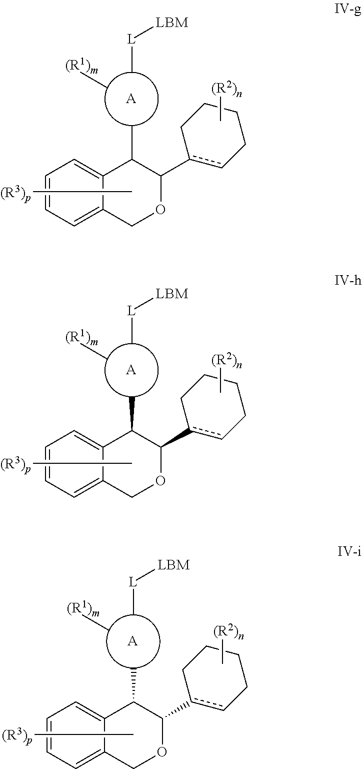 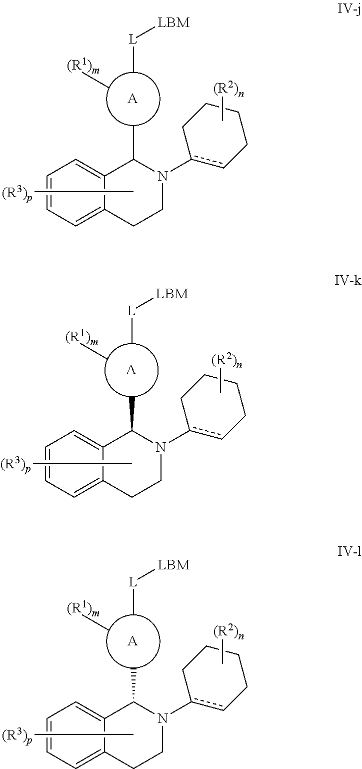
or a pharmaceutically acceptable salt thereof, wherein [symbol] represents a single or double bond.

48. The compound of claim 1 or 2, wherein the compound is a compound of one of formulas IV-m, IV-n, or IV-o: 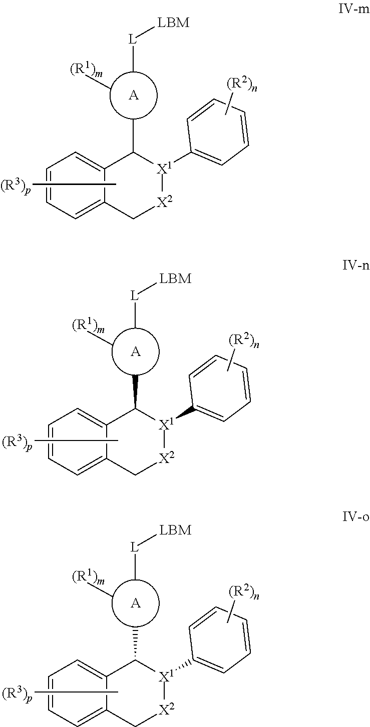
or a pharmaceutically acceptable salt thereof, provided that when X¹ is CH or N then X² is other than CH₂.

49. The compound of claim 1 or 2, wherein the compound is a compound of formula IV-m-A, IV-n-A, IV-o-A, IV-m-B, IV-n-B, IV-o-B, IV-m-C, IV-n-C, or IV-o-C: 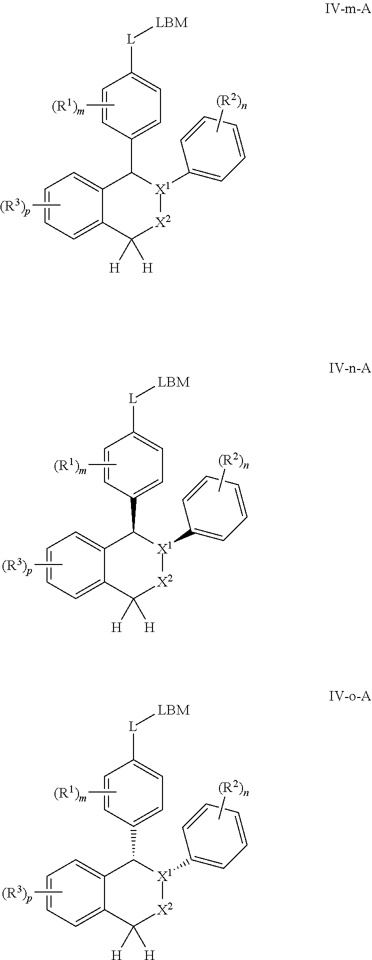 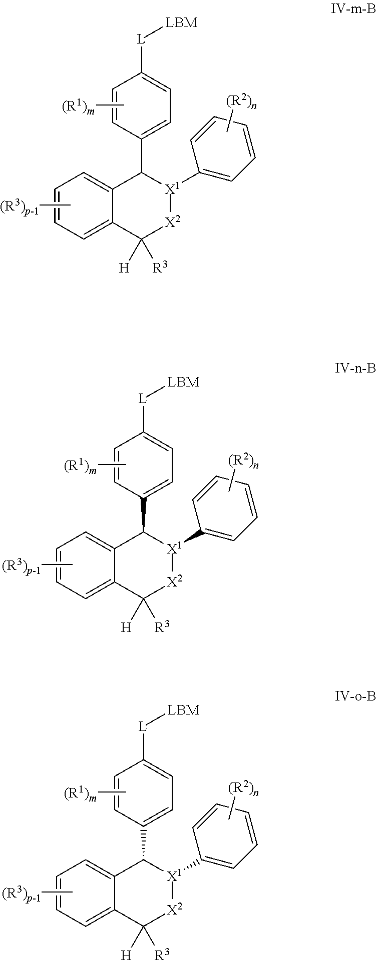 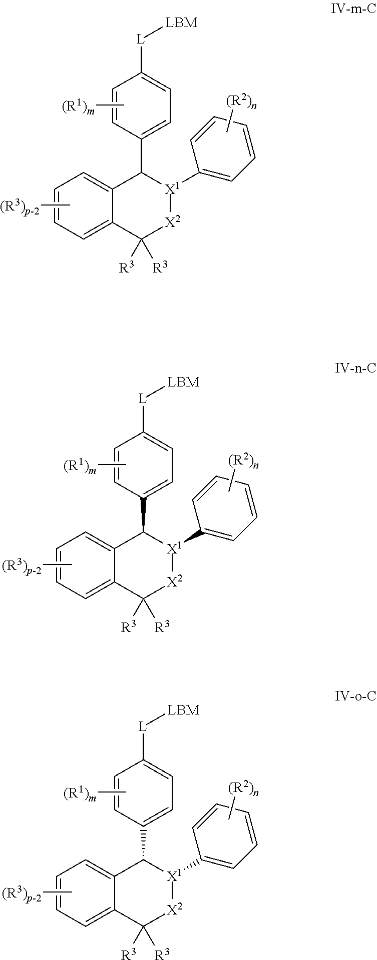
or a pharmaceutically acceptable salt thereof, provided that when the compound is of formula IV-m-A, IV-n-A or IV-o-A and X¹ is CH or N then X² is other than CH₂.

50. The compound of claim 1 or 2, wherein the compound is a compound of one of formulas IV-s, IV-t, IV-u, IV-v, IV-w, or IV-x: 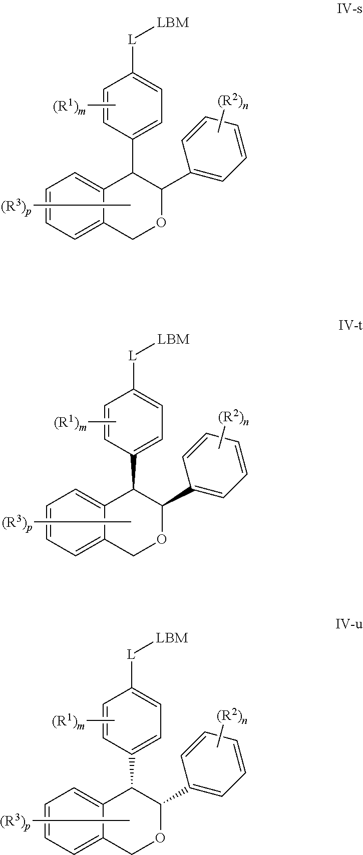 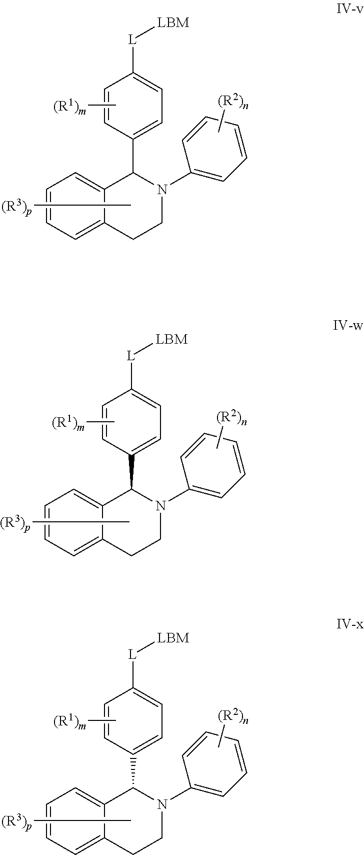
or a pharmaceutically acceptable salt thereof.

51. The compound of claim 1 or 2, wherein the compound is a compound of formula IV-aa, IV-bb, IV-cc, IV-dd, IV-ee, IV-ff, IV-gg, IV-hh, IV-ii, IV-jj, IV-kk, or IV-ll: 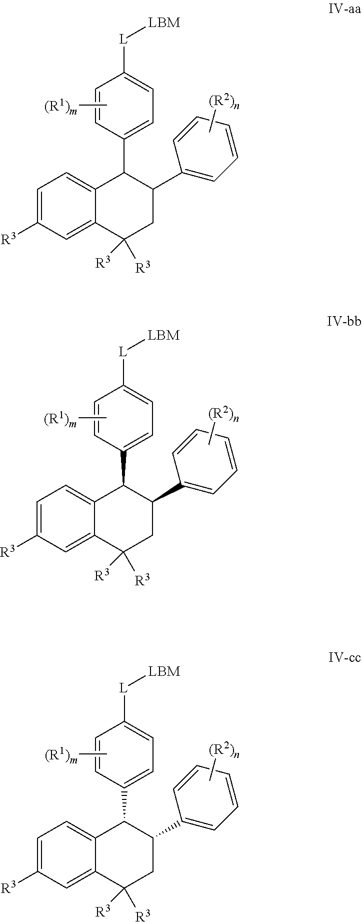 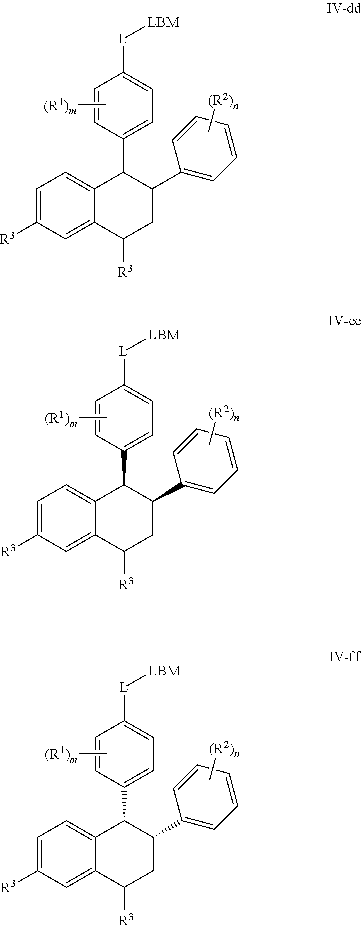 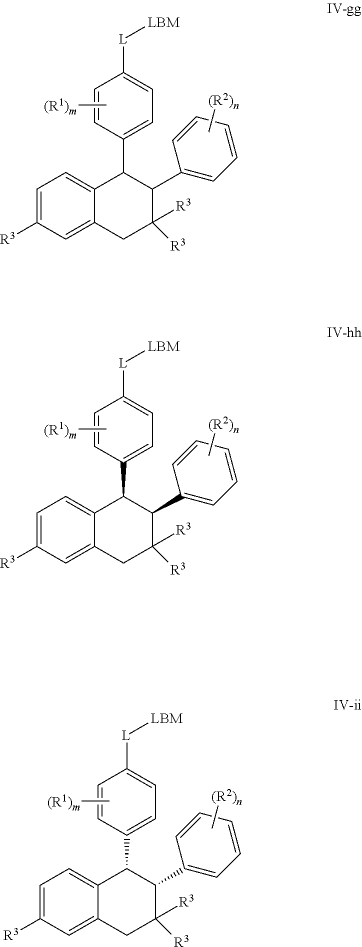 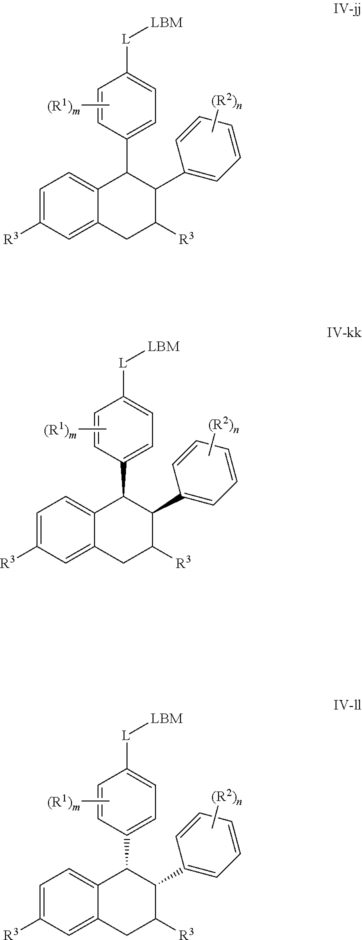
or a pharmaceutically acceptable salt thereof.

52. The compound of claim 1 or 2, wherein the compound is a compound of one of formulas V-a, V-b, or V-c: 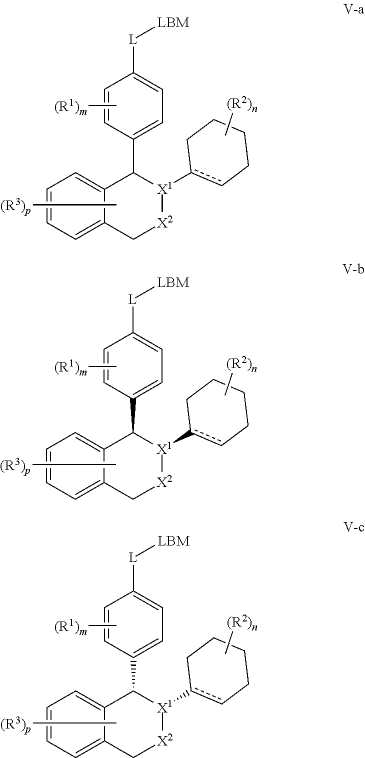
or a pharmaceutically acceptable salt thereof, wherein [symbol] represents a single or double bond.

53. The compound of claim 1 or 2, wherein the compound is a compound of one of formulas V-d, V-e, V-f, V-g, V-h, V-i, V-j, V-k, or V-l: 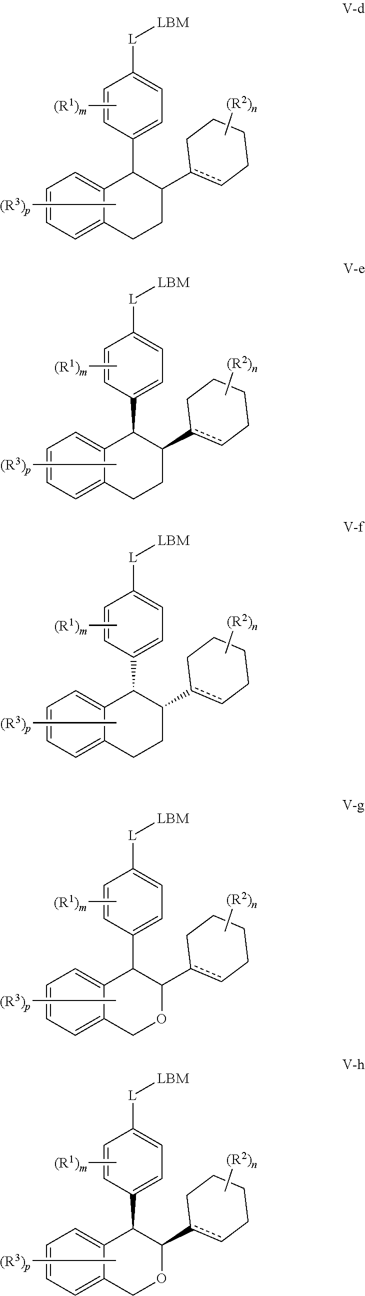 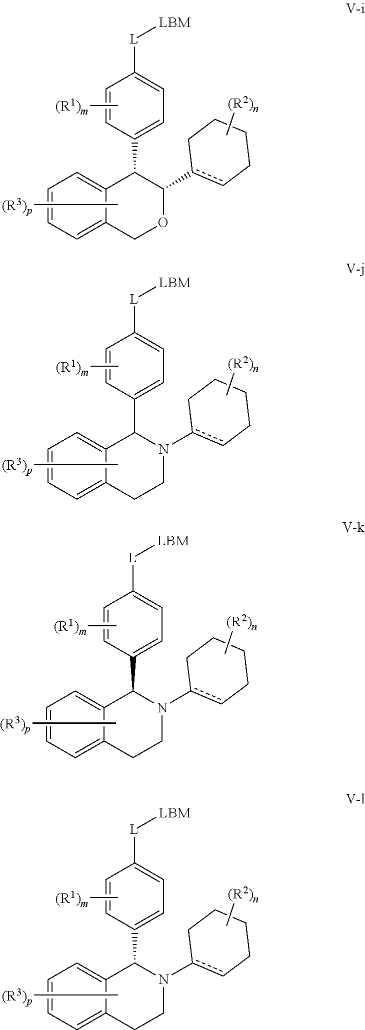
or a pharmaceutically acceptable salt thereof, wherein [symbol] represents a single or double bond.

54. The compound of claim 1 or 2, wherein the compound is a compound of one of formulas VI-a, VI-b, or VI-c: 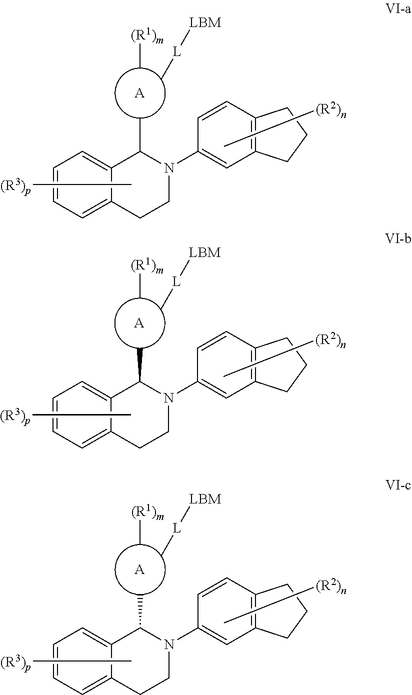
or a pharmaceutically acceptable salt thereof, wherein each of Ring A, R¹, R², R³, m, n, p, L, and LBM is as defined in claims and classes and subclasses herein.

55. The compound of claim 1 or 2, wherein the compound is a compound of one of formulas VI-d, VI-e, or VI-f: 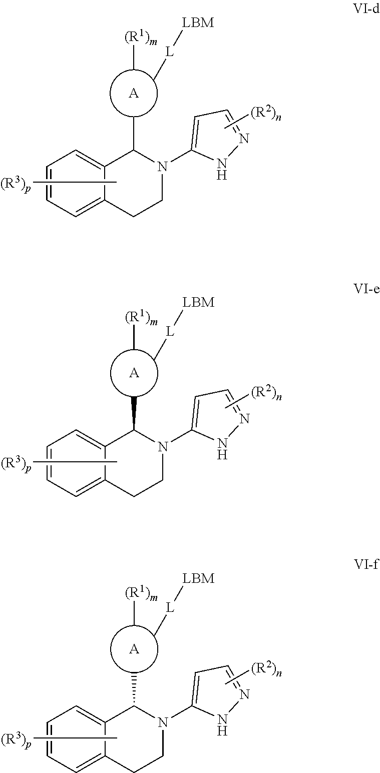
or a pharmaceutically acceptable salt thereof, wherein each of Ring A, R¹, R², R³, m, n, p, L, and LBM is as defined in claims and classes and subclasses herein.

56. The compound of claim 1 or 2, wherein the compound is a compound of one of formulas VI-g, VI-h, VI-i, VI-j, VI-k, or VI-l: 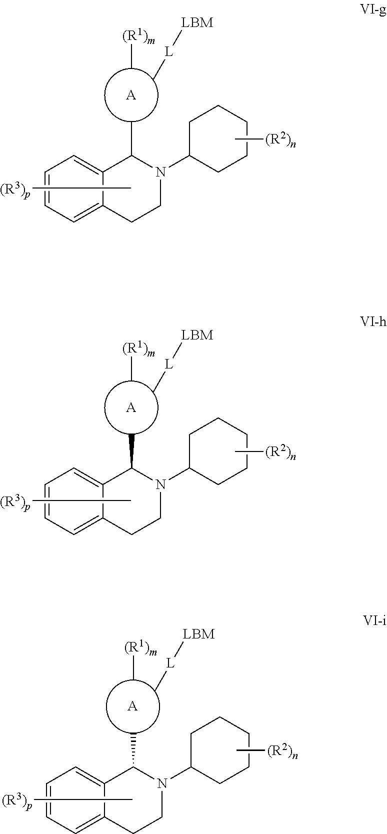 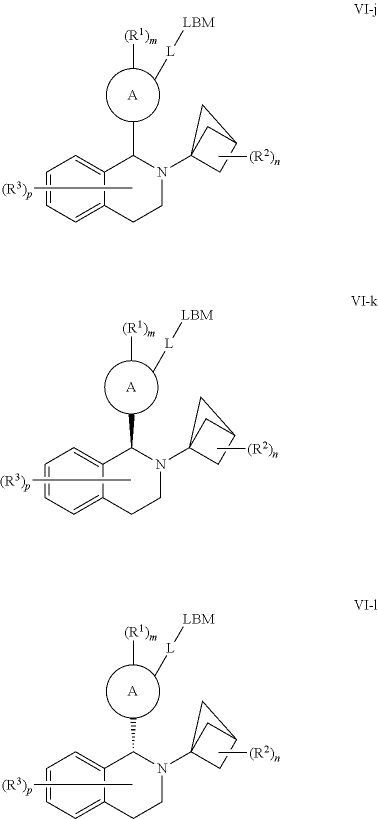
or a pharmaceutically acceptable salt thereof, wherein each of Ring A, R¹, R², R³, m, n, p, L, and LBM is as defined in claims and classes and subclasses herein.

57. The compound of claim 1 or 2, wherein the compound is a compound of one of formulas VI-m, VI-n, or VI-o: 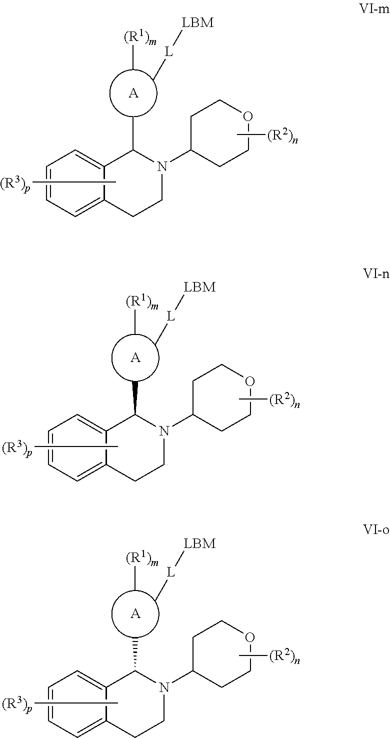
or a pharmaceutically acceptable salt thereof, wherein each of Ring A, R¹, R², R³, m, n, p, L, and LBM is as defined in claims and classes and subclasses herein.

58. The compound of claim 1 or 2, wherein the compound is a compound of one of formulas II-j-2, II-k-2, or II-l-2; 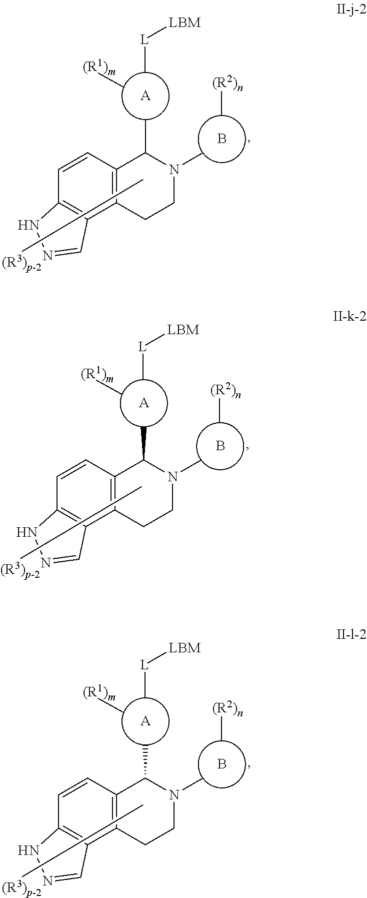
or a pharmaceutically acceptable salt thereof, wherein each of Ring A, Ring B, R¹, R², R³, m, n, p, L, and LBM is as defined in claims and classes and subclasses herein.

59. The compound of claim 1 or 2, wherein the compound is a compound of one of formulas VII-a, VII-b, VII-c, VII-d, VII-e, or VII-f: 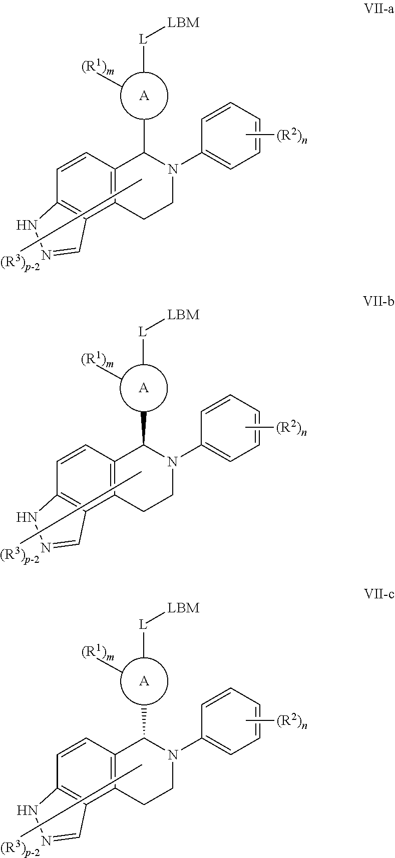 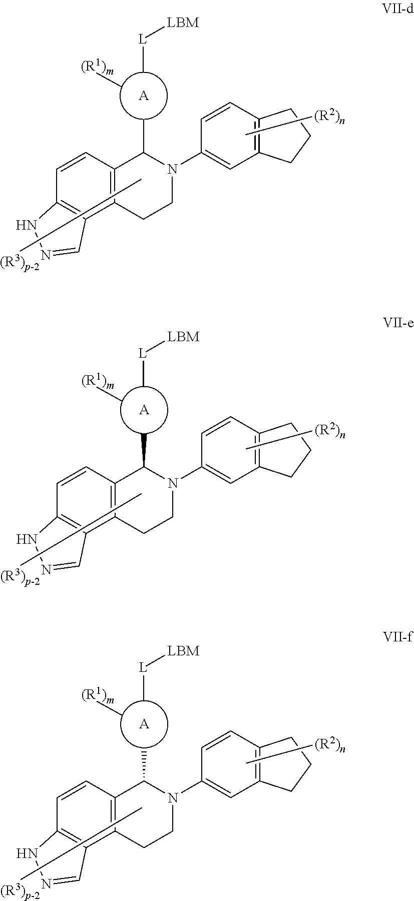
or a pharmaceutically acceptable salt thereof, wherein each of Ring A, R¹, R², R³, m, n, p, L, and LBM is as defined in claims and classes and subclasses herein.

60. The compound of claim 1 or 2, wherein the compound is a compound of one of formulas VII-g, VII-h, or VII-i: 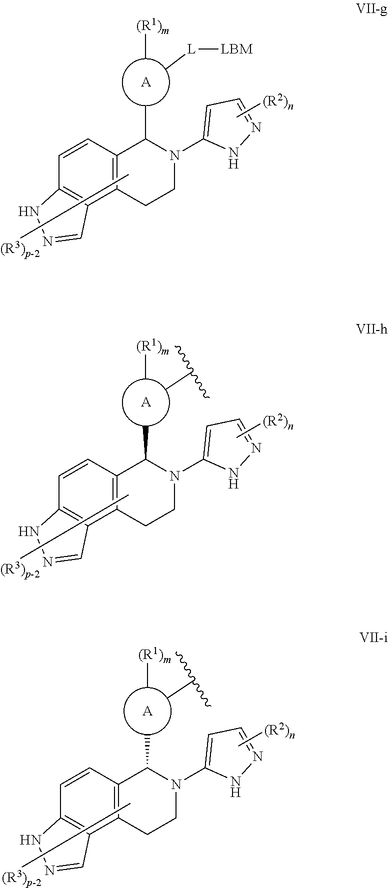
or a pharmaceutically acceptable salt thereof, wherein each of Ring A, R¹, R², R³, m, n, p, L, and LBM is as defined in claims and classes and subclasses herein.

61. The compound of claim 1 or 2, wherein the compound is a compound of one of formulas VII-j, VII-k, VII-l, VII-m, VII-n, or VII-o: 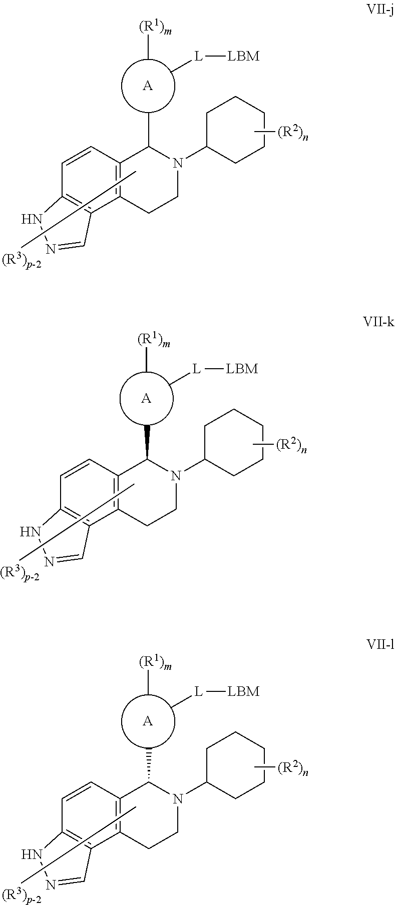 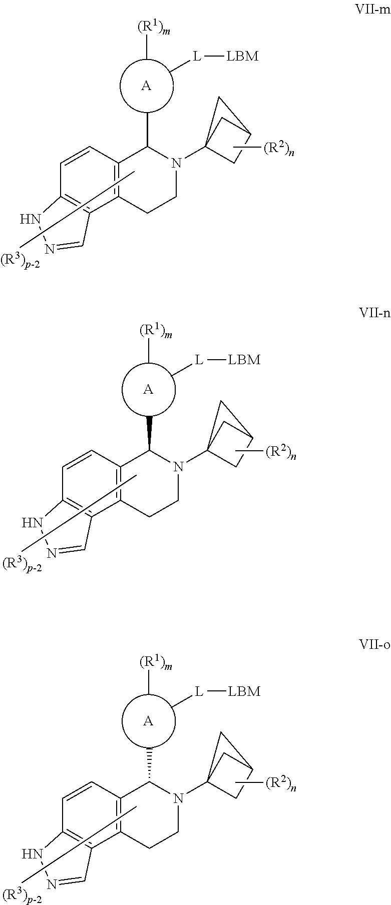
or a pharmaceutically acceptable salt thereof, wherein each of Ring A, R¹, R², R³, m, n, p, L, and LBM is as defined in claims and classes and subclasses herein.

62. The compound of claim 1 or 2, wherein the compound is a compound of one of formulas VII-p, VII-q, or VII-r, respectively: 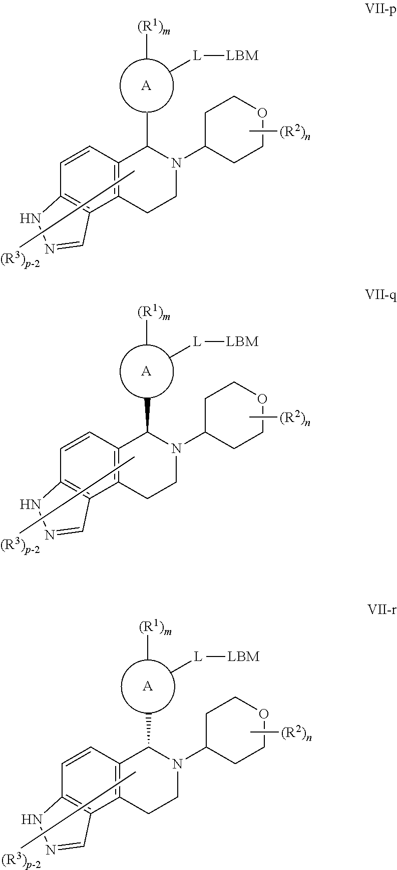
or a pharmaceutically acceptable salt thereof, wherein each of Ring A, R¹, R², R³, m, n, p, L, and LBM is as defined in claims and classes and subclasses herein.

63. The compound of claim 1 or 2, wherein the compound is a compound of one of formulas VIII-d, VIII-e, and VIII-f: 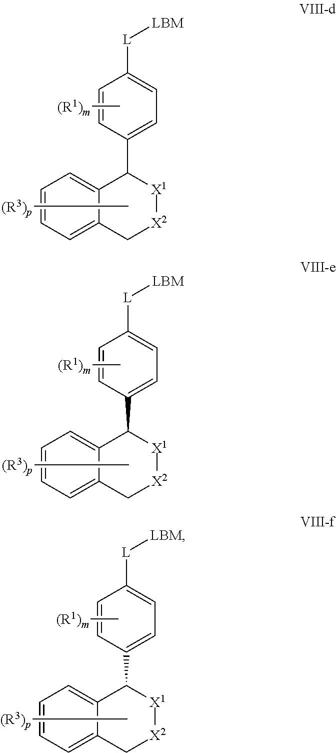
or a pharmaceutically acceptable salt thereof.

64. The compound of claim 1 or 2, wherein the compound is a compound of one of formulas VIII-g, VIII-h, VIII-i, VIII-j, VIII-k or VIII-l: 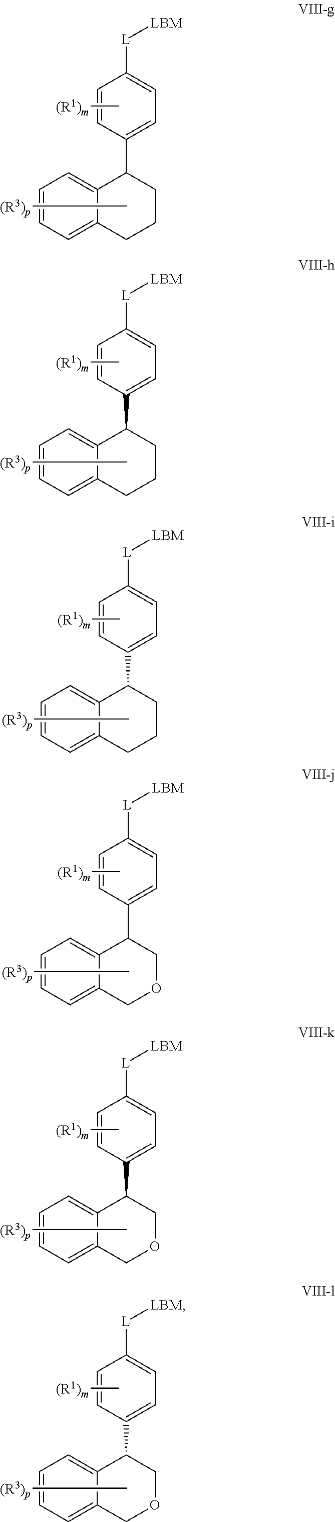
or a pharmaceutically acceptable salt thereof.

65. The compound of claim 1 or 2, wherein the compound is a compound of one of formulas VIII-m, VIII-n, VIII-o, VIII-p, VIII-q, VIII-r, VIII-s, VIII-t, or VIII-u: 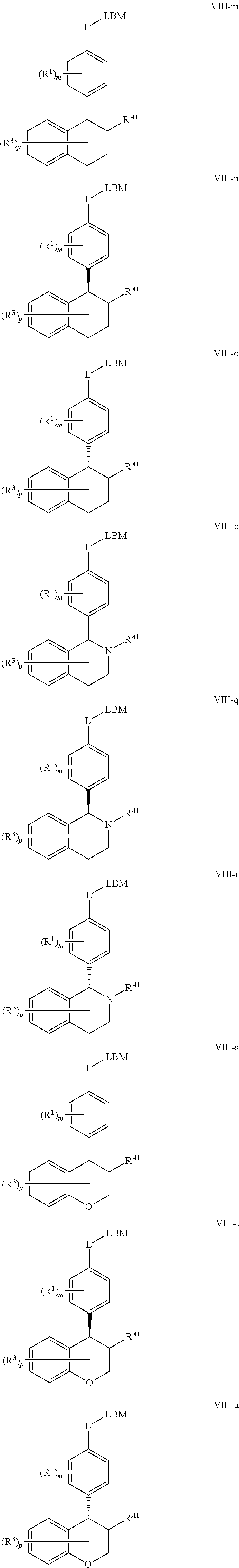
or a pharmaceutically acceptable salt thereof.

66. The compound of claim 1 or 2, wherein the compound is a compound of one of formulas IX-d, IX-e, IX-f: 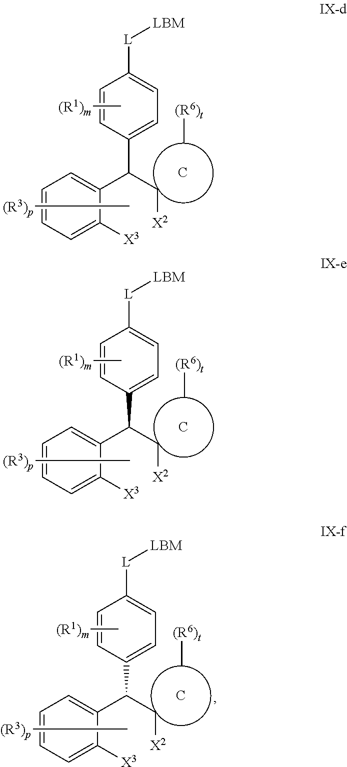
or a pharmaceutically acceptable salt thereof.

67. The compound of claim 1 or 2, wherein the compound is a compound of one of formulas IX-g, IX-h, IX-i, IX-j, IX-k, IX-l, IX-m, IX-n, IX-o: 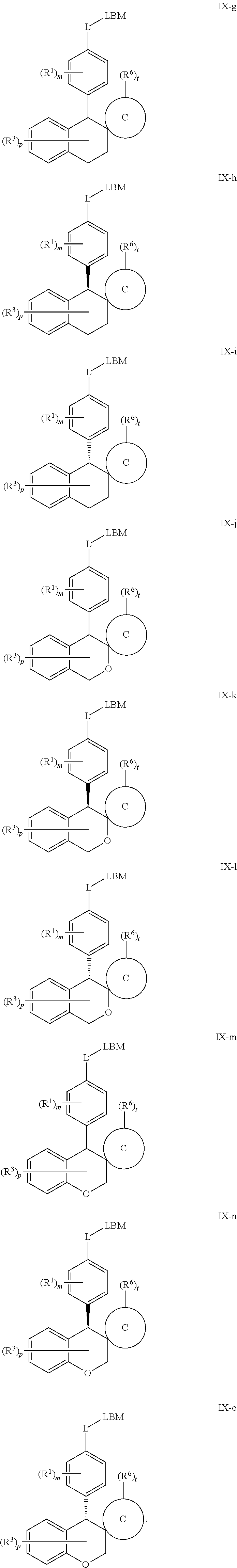
or a pharmaceutically acceptable salt thereof.

68. The compound of any one of claims 1-67, wherein Ring C together with its R⁶ substituents is selected from:

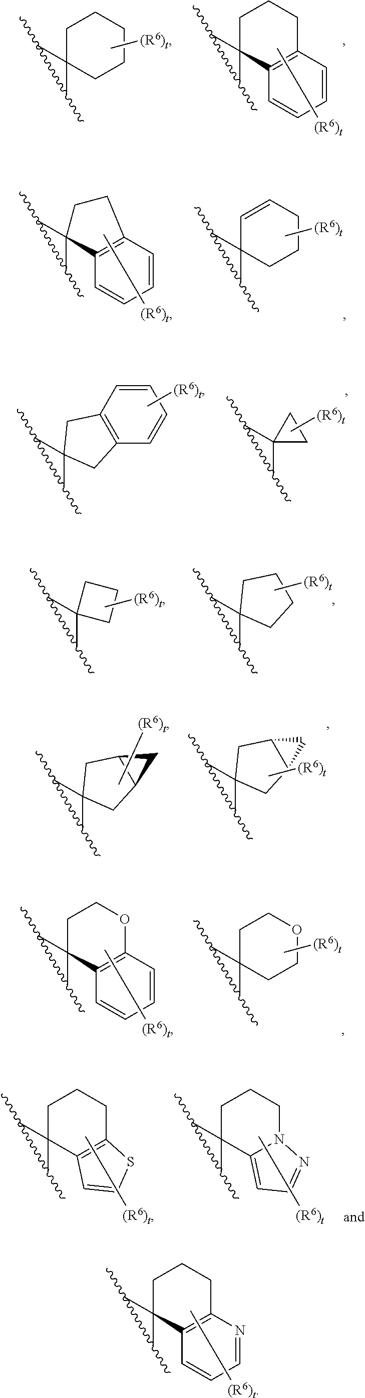

69. The compound of any one of claims 1-68, wherein X¹ is selected from CH and C(R^{A1}).

70. The compound of any one of claims 1-69, wherein X² is selected from O, CH₂, CH(R^{A3}), and C(R^{A3})₂, provided that X¹ and X² are not both simultaneously heteroatoms.

71. The compound of any one of claims 1-70, wherein L is L is a bivalent, saturated or unsaturated, straight or branched C₁₋₅₀ hydrocarbon chain, wherein 0-6 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

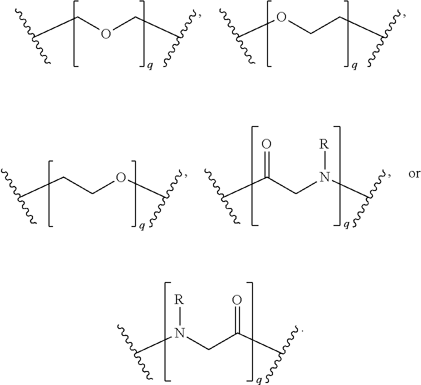

72. The compound of any one of claims 1-70, wherein L is a bivalent, saturated or unsaturated, straight or branched C₃₋₅ hydrocarbon chain, wherein 0, 1, 2 or 3 methylene units of L are independently replaced by -Cy-, —CH(R)—, —C(R)₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

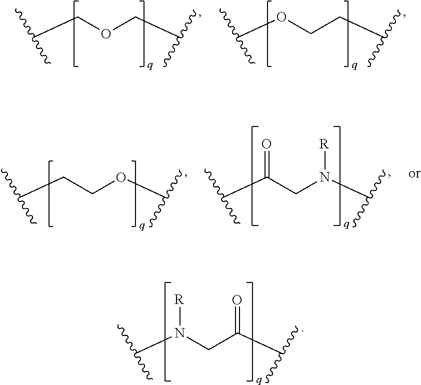

73. The compound of any one of claims 1-70, wherein L is a bivalent, saturated or unsaturated, straight or branched C₃₋₅ hydrocarbon chain, wherein 1, 2 or 3 methylene units of L are independently replaced by -Cy-, —CH(R)—, —C(R)₂—, —O— or —NR—.

74. The compound of any one of claims 1-70, wherein L is a bivalent, saturated or unsaturated, straight or branched C₃₋₅ hydrocarbon chain, wherein 1, 2 or 3 methylene units of L are independently replaced by -Cy- or —NR—.

75. The compound of any one of claims 1-70, wherein each -Cy- is independently an optionally substituted bivalent ring selected from a 4-7 membered saturated or partially unsaturated monocyclic carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated monocyclic heterocyclylenyl having 1-2 heteroatoms independently selected from nitrogen, oxygen, and sulfur, a 5-11 membered monocyclic saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur and a 6-10 membered bridged bicyclic saturated or partially unsaturated heterocyclylenyl having 1-2 heteroatoms independently selected from nitrogen, oxygen, and sulfur.

76. The compound of any one of claims 1-70, wherein each -Cy- is independently an optionally substituted bivalent ring selected from a 4-7 membered saturated or partially unsaturated monocyclic carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated monocyclic heterocyclylenyl containing 1-2 nitrogen atoms, a 5-11 membered monocyclic saturated or partially unsaturated spiro heterocyclylenyl containing 1-2 nitrogen atoms, an 8-10 membered bicyclic saturated or partially unsaturated heterocyclylenyl containing 1-2 nitrogen atoms, and a 6-10 membered bridged bicyclic saturated or partially unsaturated heterocyclylenyl containing 1-2 nitrogen atoms.

77. The compound of any one of claims 1-70, wherein the bivalent ring of each -Cy- is independently substituted with 0, 1 or 2 substituents independently selected from deuterium, C₁-C₄ alkyl, C₁-C₄ haloalkyl, C₃-C₇ cycloalkyl, —O—C₁-C₄ alkyl, halo, cyano, —OH, —NH₂, —N(H)(C₁-C₄ alkyl) and —N(C₁-C₄ alkyl)₂.

78. The compound of any one of claims 1-70, wherein the bivalent ring of each -Cy- is independently substituted with 0, 1 or 2 substituents independently selected from —C₁-C₄ alkyl, —C₁-C₄ haloalkyl, —C₃-C₇ cycloalkyl, —O—C₁-C₄ alkyl, halo, cyano, —OH, —NH₂, —N(H)(C₁-C₄ alkyl) and —N(C₁-C₄ alkyl)₂.

79. The compound of any one of claims 1-70, wherein the bivalent ring of each -Cy- is independently substituted with 0, 1 or 2 substituents independently selected from -Me, -Et, Pr, iPr, cyclopropyl, —CF₃, —OMe, —F, —Cl, —CN, —NH₂, —NHMe and —NMe₂.

80. The compound of any one of claims 1-70, wherein the bivalent ring of each -Cy- is independently substituted with 0, 1 or 2 substituents independently selected from -Me, —OMe and —F.

81. The compound of any one of claims 1-70, wherein the bivalent ring of each -Cy- is independently substituted with 0, 1 or 2 instances of -Me.

82. The compound of any one of claims 1-70, wherein the bivalent ring of each -Cy- is unsubstituted.

83. The compound of any one of claims 1-70, wherein the bivalent ring of each -Cy- is substituted with 1 or 2 substituents.

84. The compound of any one of claims 1-70, wherein L is

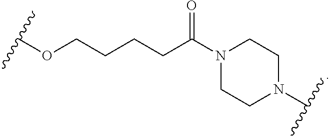 [SMILES: *OCCCCC(=O)N1CCN(*)CC1]

85. The compound of any one of claims 1-70, wherein L is selected from:

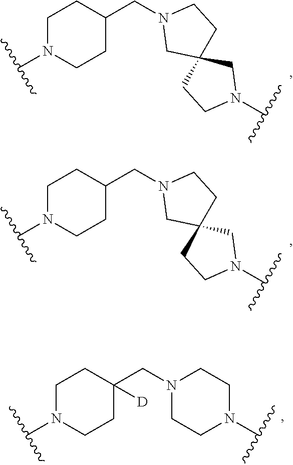 [SMILES: *N1CCC(CN2CC[C@]3(CCN(*)C3)C2)CC1 ; *N1CCC(CN2CC[C@@]3(CCN(*)C3)C2)CC1 ; *N1CCN(CC2([2H])CCN(*)CC2)CC1]

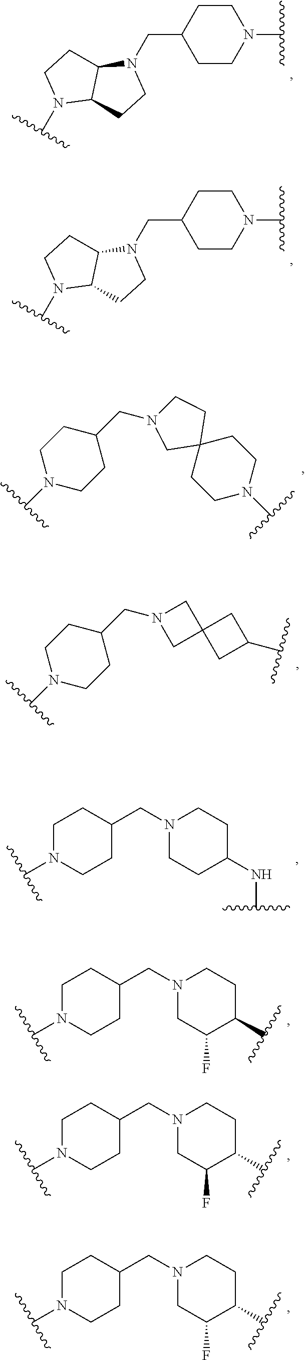

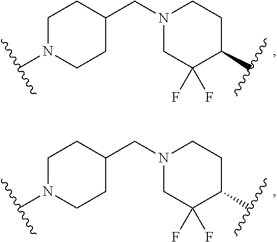 [SMILES: *[C@@H]1CCN(CC2CCN(*)CC2)CC1(F)F ; *[C@H]1CCN(CC2CCN(*)CC2)CC1(F)F]

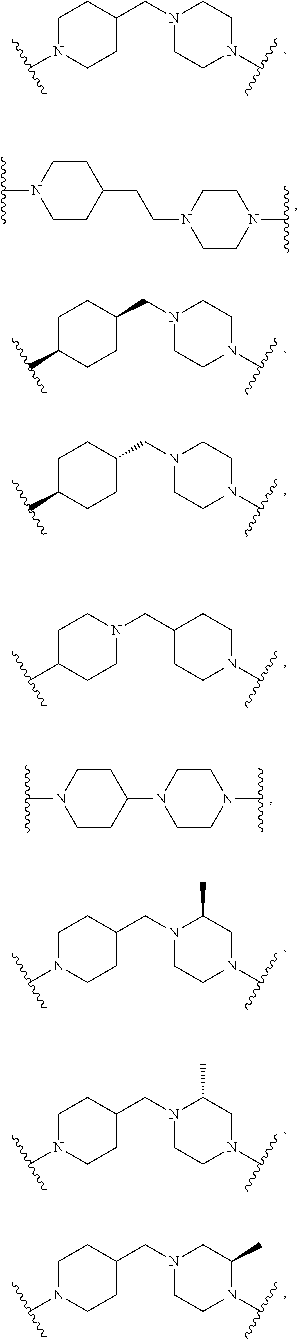

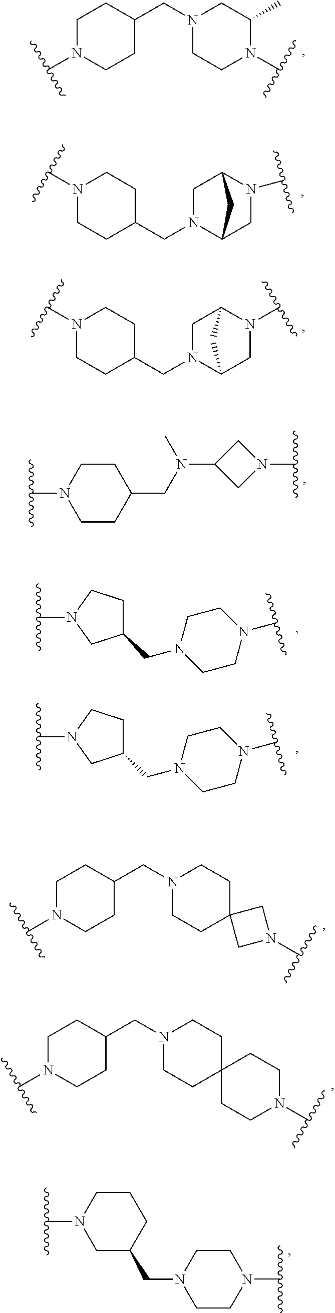

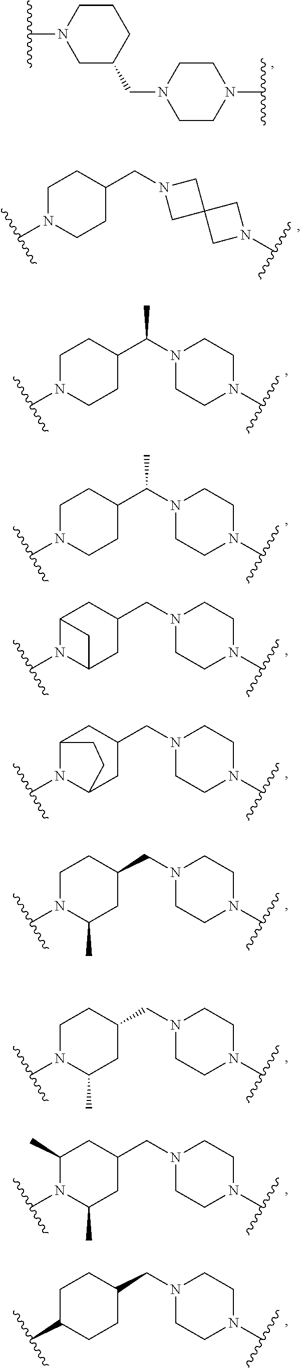

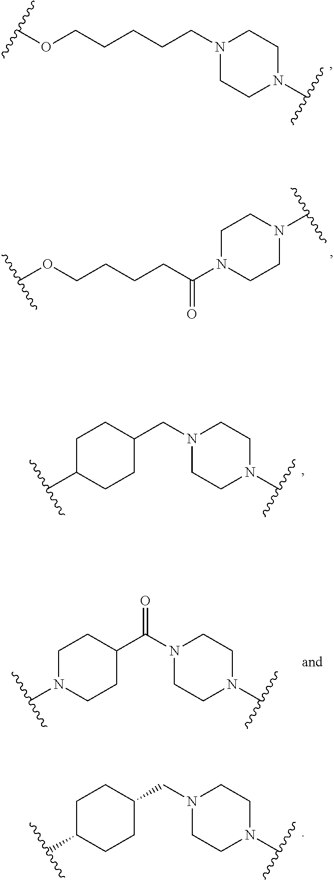 [SMILES: *OCCCCCN1CCN(*)CC1 ; *OCCCCC(=O)N1CCN(*)CC1 ; *C1CCC(CN2CCN(*)CC2)CC1 ; *N1CCC(C(=O)N2CCN(*)CC2)CC1 ; *N1CCN(C[C@H]2CC[C@@H](*)CC2)CC1]

86. The compound of any one of claims 1-85, wherein LBM is selected from:

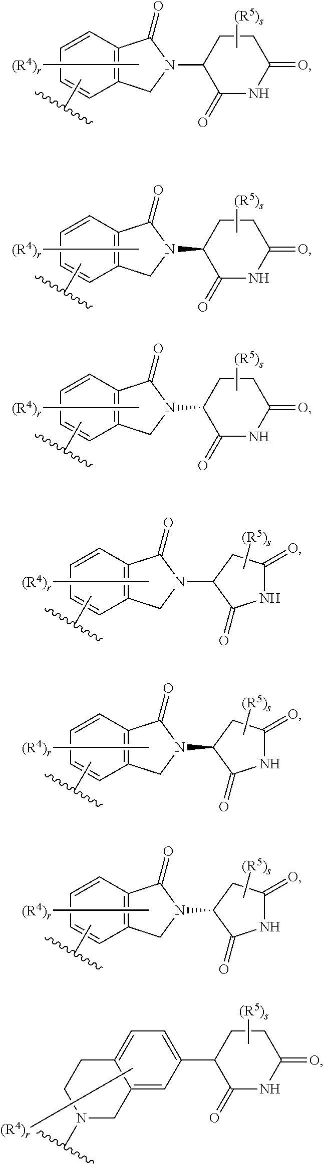

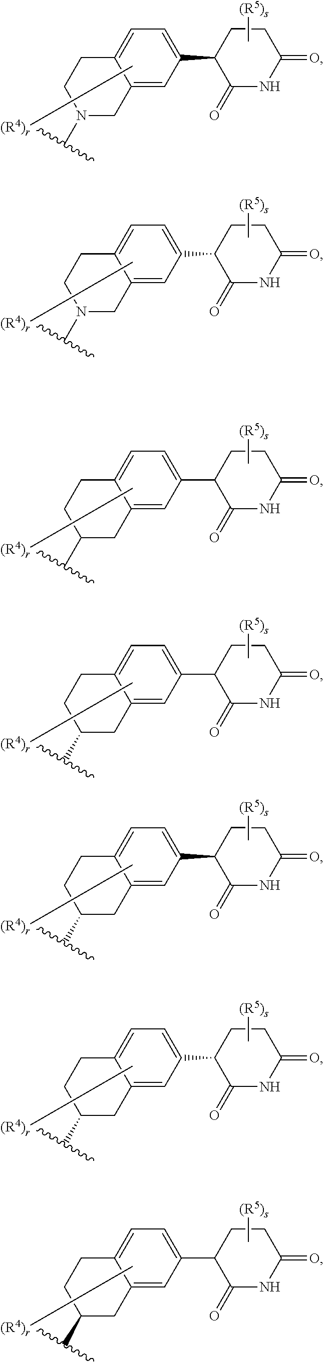

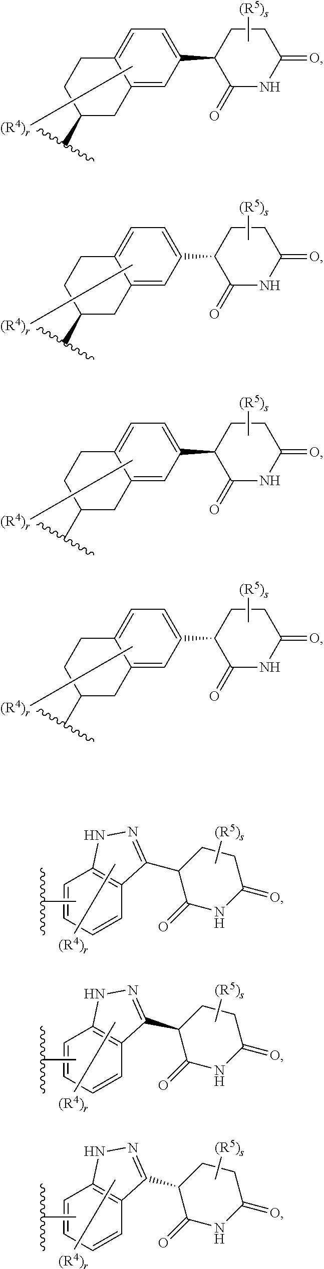

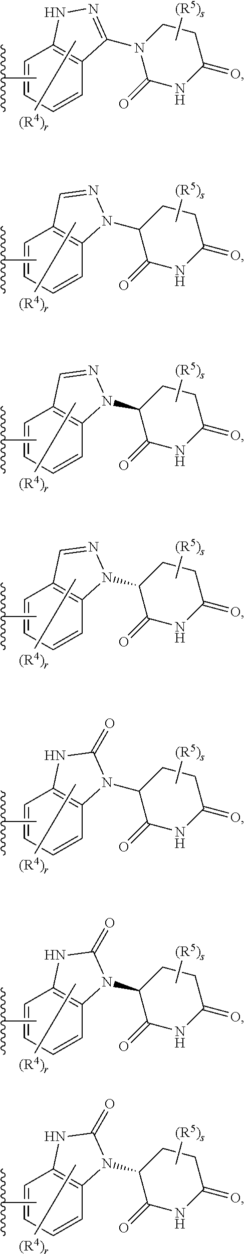

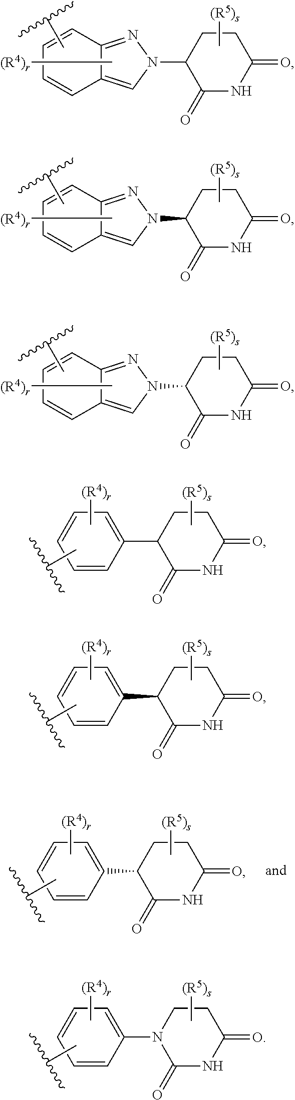

87. The compound of any one of claims 1-85, wherein LBM is selected from:

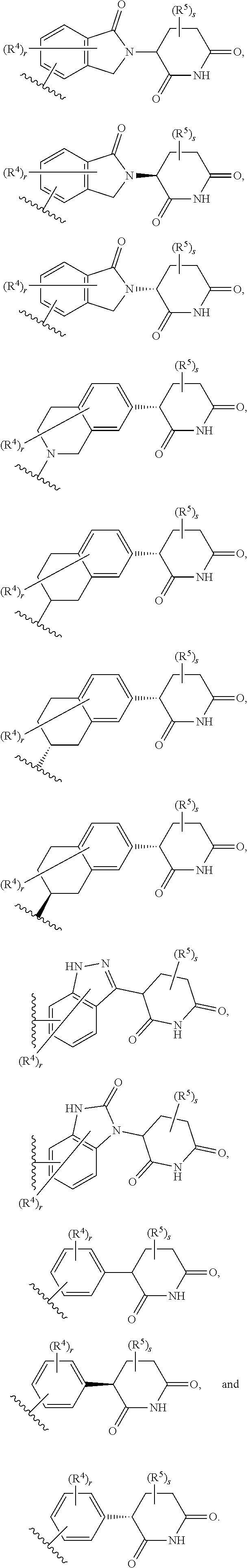

88. The compound of any one of claims 1-85, wherein LBM is selected from

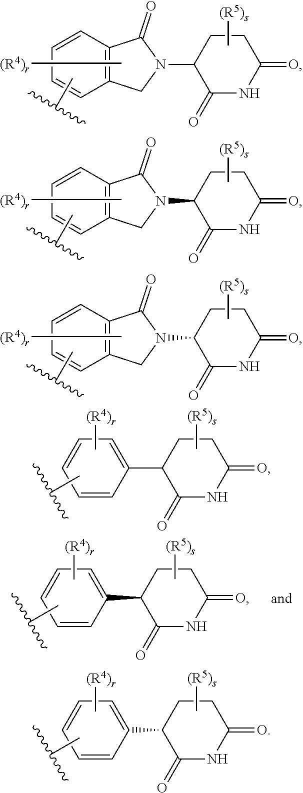

89. The compound of any one of claims 1-88, wherein each R⁴ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

90. The compound of any one of claims 1-88, wherein each R⁴ is independently selected from -Me, -Et, —F, —Cl, —CF₃, —CN, —OH, —OMe, —NH₂, —NHMe and —NMe₂.

91. The compound of any one of claims 1-88, wherein each R⁵ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

92. The compound of any one of claims 1-88, wherein each R⁵ is independently selected from -Me, -Et, —F, —Cl, —CF₃, —CN, —OH, —OMe, —NH₂, —NHMe and —NMe₂.

93. The compound of any one of claims 1-92, wherein each R⁵ is independently selected from -Me and —F.

94. The compound of any one of claims 1-93, wherein r is 0, 1 or 2.

95. The compound of any one of claims 1-94, wherein s is 0, 1 or 2.

96. The compound of any one of claims 1-85, wherein LBM is selected from

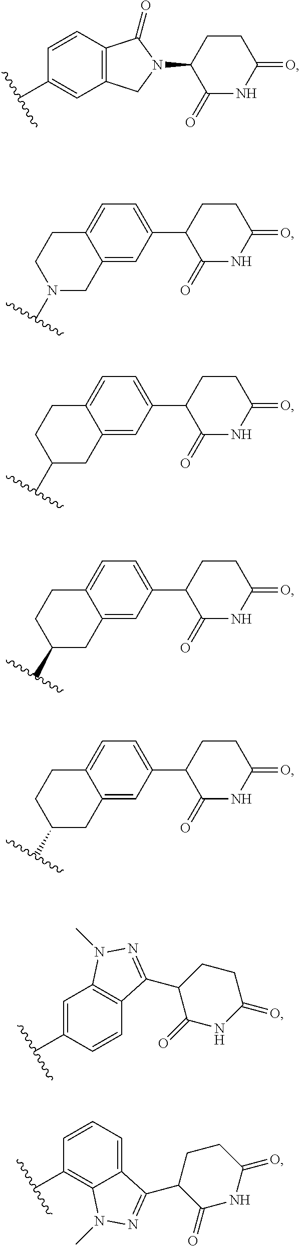

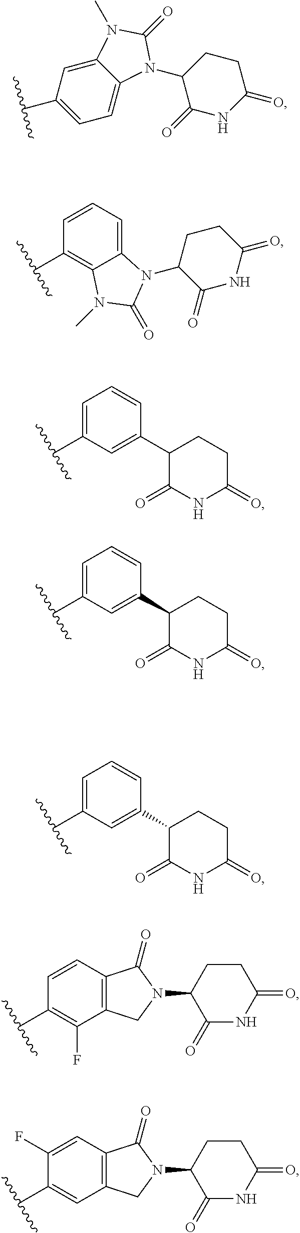

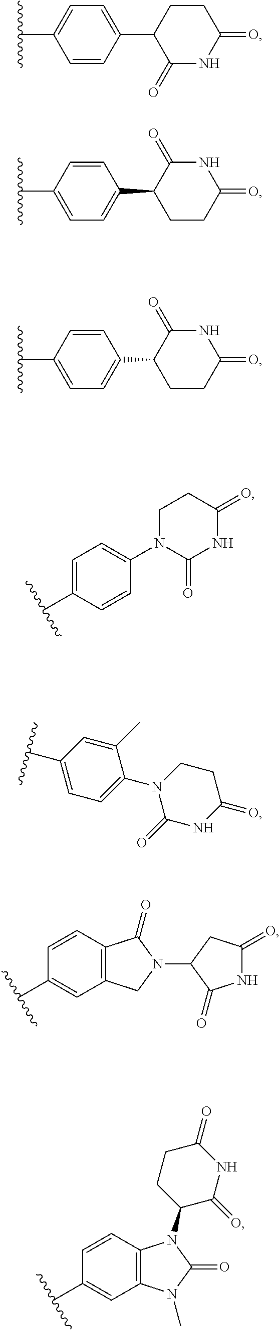

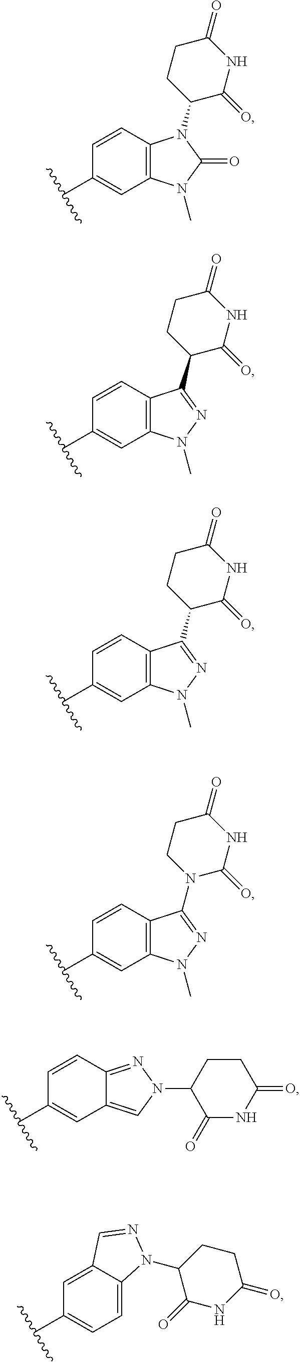

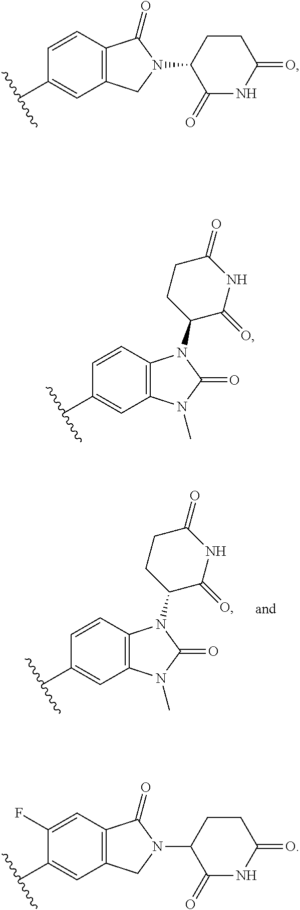 [SMILES: *c1ccc2c(c1)CN([C@@H]1CCC(=O)NC1=O)C2=O ; *c1ccc2c(c1)n(C)c(=O)n2[C@H]1CCC(=O)NC1=O ; *c1ccc2c(c1)n(C)c(=O)n2[C@@H]1CCC(=O)NC1=O ; *c1cc2c(cc1F)C(=O)N(C1CCC(=O)NC1=O)C2]

97. The compound of any one of claims 1-85, wherein LBM is selected from:

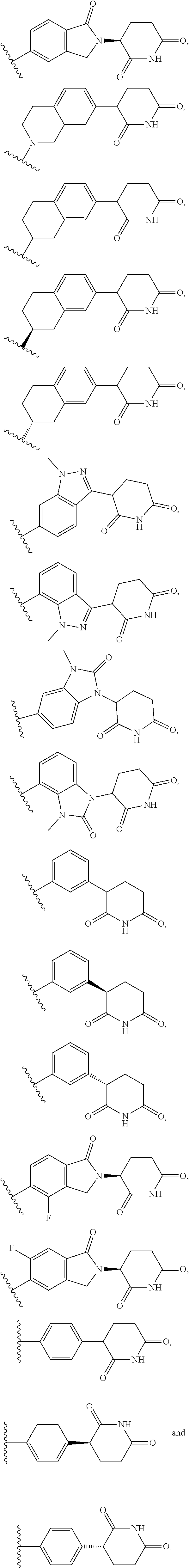

98. The compound of any one of claims 1-85, wherein LBM is selected from

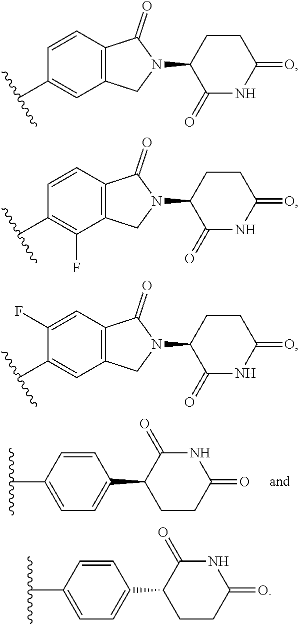

99. The compound of any one of claims 1-85, wherein LBM is selected from

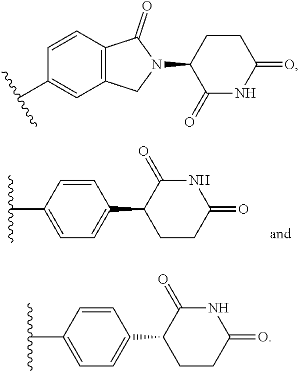

100. The compound of any one of claims 1-85, wherein LBM is

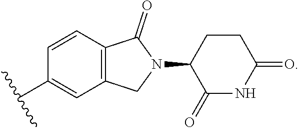 [SMILES: *c1ccc2c(c1)CN([C@H]1CCC(=O)NC1=O)C2=O]

101. The compound of any one of claims 1-100, wherein each R¹ is independently selected from deuterium, C₁₋₆ aliphatic chain substituted with 0-3 instances of halo, halogen, —CN, —SR, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain substituted with 0-3 instances of halo.

102. The compound of any one of claims 1-100, wherein each R′ is independently selected from -Me, -Et, —F, —Cl, —SCF₃, —OCF₃, —CF₃, —CN, —OH, —OMe, —NH₂, —NHMe and —NMe₂.

103. The compound of any one of claims 1-100, wherein each R′ is independently selected from -Me and —F.

104. The compound of any one of claims 1-103, wherein each R₂ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain.

105. The compound of any one of claims 1-103, wherein each R₂ is independently selected from -Me, -Et, —F, —Cl, —CF₃, —CN, —OH, —OMe, —NH₂, —NHMe and —NMe₂.

106. The compound of any one of claims 1-103, wherein each R₂ is independently selected from —F, —Cl and —CF₃.

107. The compound of any one of claims 1-106, wherein m is 0, 1 or 2.

108. The compound of any one of claims 1-107, wherein n is 0, 1 or 2.

109. The compound of any one of claims 1-108, wherein each R³ is independently selected from deuterium, C₁₋₆ aliphatic chain substituted with 0-3 instances of halo, halogen, —CN, —OR, —NR₂, —S(O)₂R, —S(O)₂NR₂, —S(O)R, —S(O)NR₂, —S(O)(NR)R, —C(O)OR, —C(O)NR₂, —C(O)N(R)OR, —OC(O)NR₂, —N(R)C(O)OR, —N(R)C(O)R, —N(R)C(O)NR₂, —N(R)S(O)₂NR₂ and —N(R)S(O)₂R, wherein R is H or a C₁₋₆ aliphatic chain or two R³ groups are taken together to form

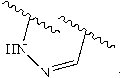 [SMILES: */C=N\N*]

110. The compound of any one of claims 1-108, wherein each R³ is independently selected from -Me, -Et, —F, —Cl, —CF₃, —CO₂H, —CN, —OH, —OMe, —NH₂, —NHMe and —NMe₂ or two R³ groups are taken together to form

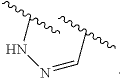 [SMILES: */C=N\N*]

111. The compound of any one of claims 1-108, wherein each R³ is independently selected from —F, —OH, and —CO₂H.

112. The compound of any one of claims 1-108, wherein each R³ is independently selected from -Me, —F, —OH, and —CO₂H

113. The compound of any one of claims 1-108, wherein each R³ is independently selected from —F and —OH.

114. The compound of any one of claims 1-108, wherein each R³ is independently selected from -Me and —F.

115. The compound of any one of claims 1-108, wherein two R³ groups are taken together to form

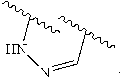 [SMILES: */C=N\N*]

116. The compound of any one of claims 1-115, wherein p is 0, 1 or 2.

117. The compound of any one of claims 1-116, wherein each R^{e} is independently selected from -Me, -Et, —F, —Cl, —CF₃, —CO₂H, —CN, —OH, —OMe, —NH₂, —NHMe and —NMe₂

118. The compound of any one of claims 1-116, wherein each R⁶ is independently selected from -Me, and —F.

119. The compound of any one of claims 1-118, wherein t is 0, 1 or 2.

120. The compound of any one of claims 1-119, wherein the compound is a compound selected from: 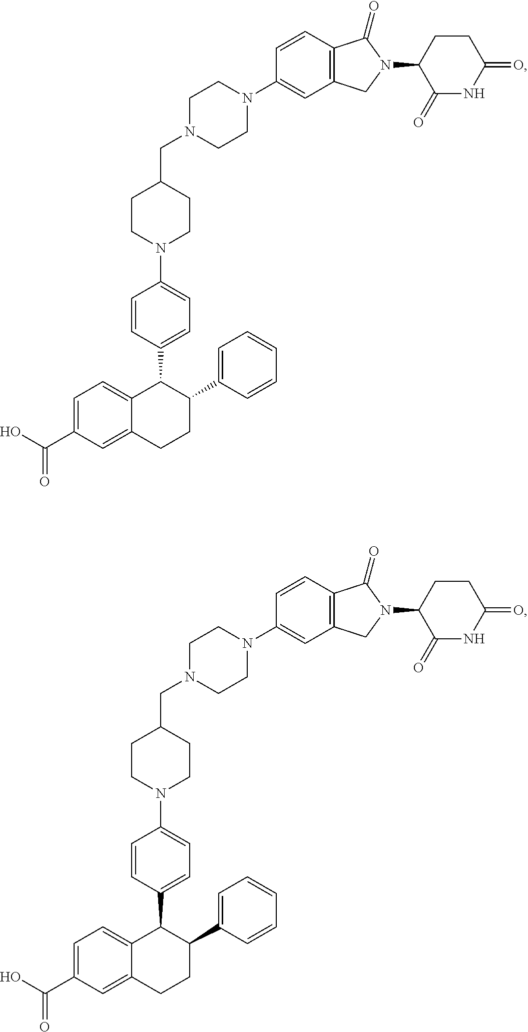 [SMILES: O=C1CC[C@H](N2Cc3cc(N4CCN(CC5CCN(c6ccc([C@H]7c8ccc(C(=O)O)cc8CC[C@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(C(=O)O)cc8CC[C@@H]7c7ccccc7)cc6)CC5)CC4)ccc3C2=O)C(=O)N1] 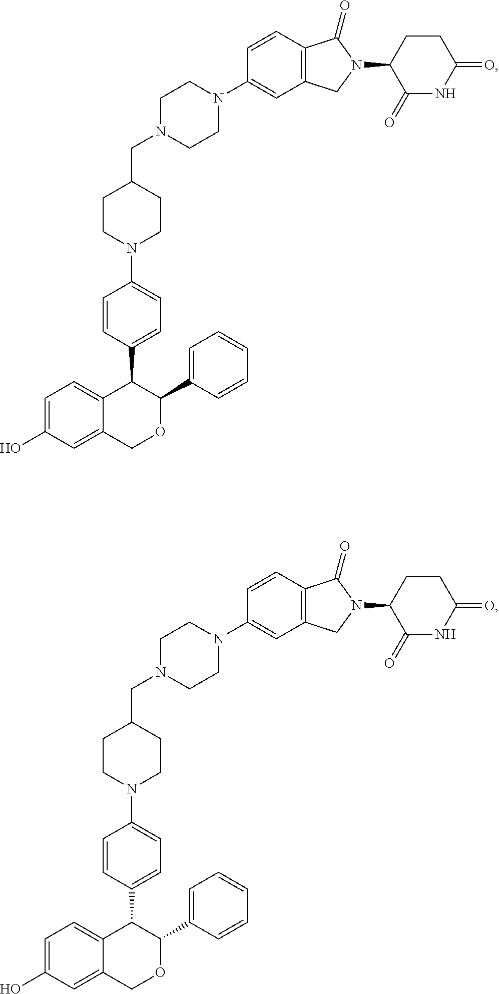 [SMILES: O=C1CC[C@H](N2Cc3cc(N4CCN(CC5CCN(c6ccc([C@@H]7c8ccc(O)cc8CO[C@@H]7c7ccccc7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7c7ccccc7)cc6)CC5)CC4)ccc3C2=O)C(=O)N1] 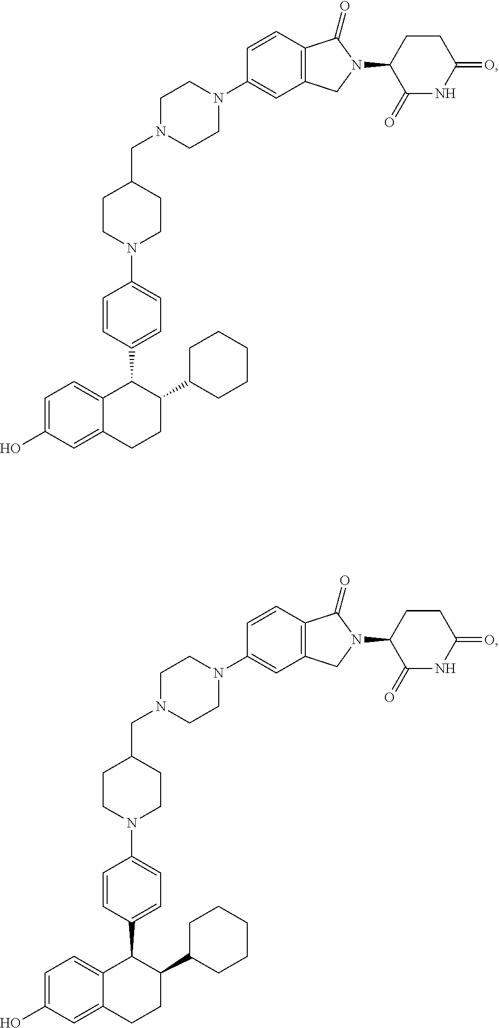 [SMILES: O=C1CC[C@H](N2Cc3cc(N4CCN(CC5CCN(c6ccc([C@H]7c8ccc(O)cc8CC[C@H]7C7CCCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCCCC7)cc6)CC5)CC4)ccc3C2=O)C(=O)N1] 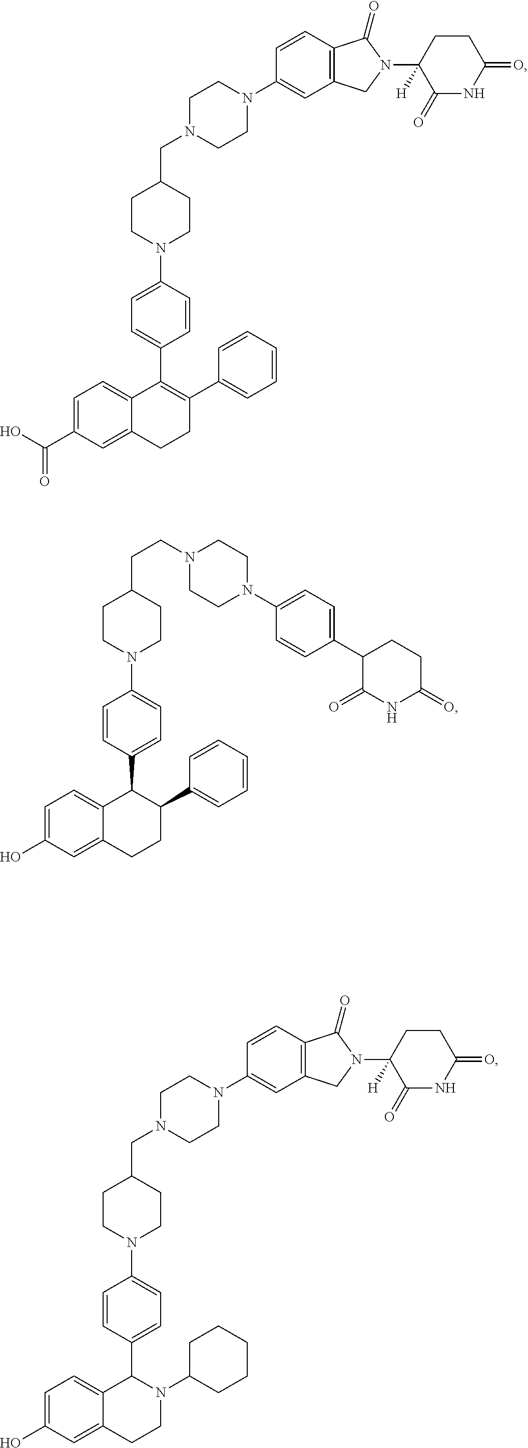 [SMILES: O=C1CC[C@H](N2Cc3cc(N4CCN(CC5CCN(c6ccc(C7=C(c8ccccc8)CCc8cc(C(=O)O)ccc87)cc6)CC5)CC4)ccc3C2=O)C(=O)N1 ; O=C1CCC(c2ccc(N3CCN(CCC4CCN(c5ccc([C@@H]6c7ccc(O)cc7CC[C@@H]6c6ccccc6)cc5)CC4)CC3)cc2)C(=O)N1 ; O=C1CC[C@H](N2Cc3cc(N4CCN(CC5CCN(c6ccc(C7c8ccc(O)cc8CCN7C7CCCCC7)cc6)CC5)CC4)ccc3C2=O)C(=O)N1] 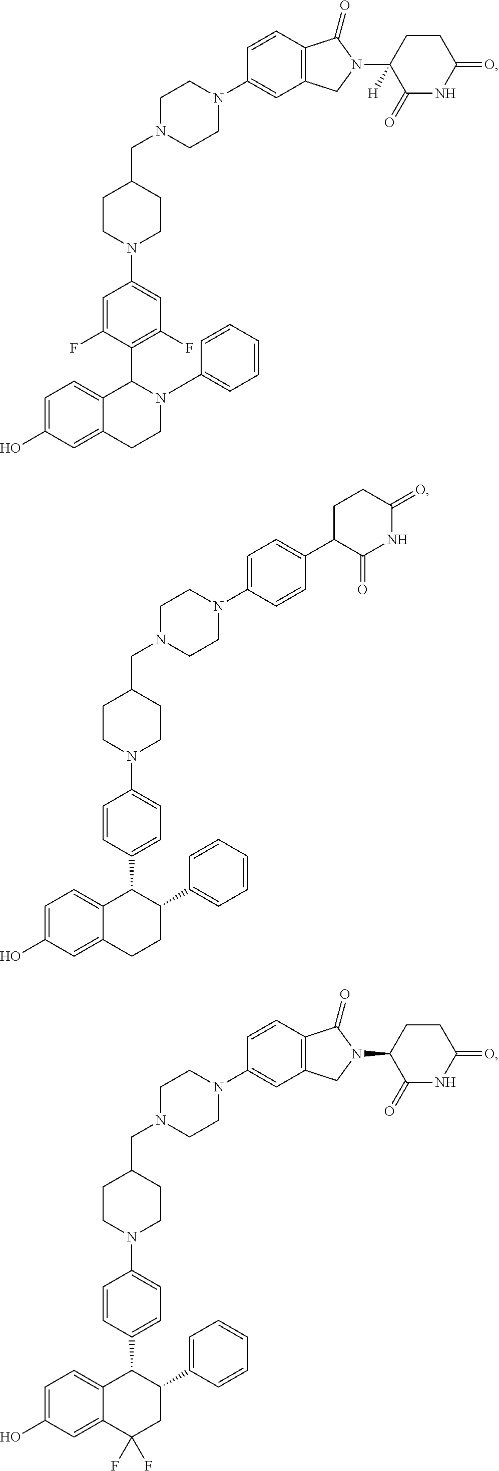 [SMILES: O=C1CC[C@H](N2Cc3cc(N4CCN(CC5CCN(c6cc(F)c(C7c8ccc(O)cc8CCN7c7ccccc7)c(F)c6)CC5)CC4)ccc3C2=O)C(=O)N1 ; O=C1CCC(c2ccc(N3CCN(CC4CCN(c5ccc([C@H]6c7ccc(O)cc7CC[C@H]6c6ccccc6)cc5)CC4)CC3)cc2)C(=O)N1 ; O=C1CC[C@H](N2Cc3cc(N4CCN(CC5CCN(c6ccc([C@H]7c8ccc(O)cc8C(F)(F)C[C@H]7c7ccccc7)cc6)CC5)CC4)ccc3C2=O)C(=O)N1] 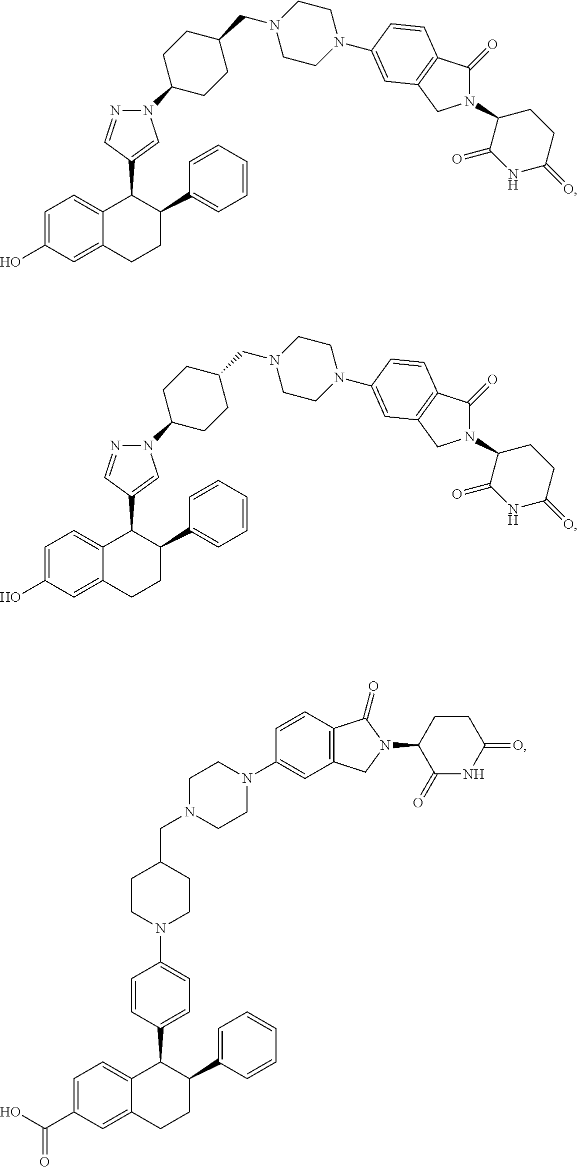 [SMILES: O=C1CC[C@H](N2Cc3cc(N4CCN(C[C@H]5CC[C@@H](n6cc([C@@H]7c8ccc(O)cc8CC[C@@H]7c7ccccc7)cn6)CC5)CC4)ccc3C2=O)C(=O)N1 ; O=C1CC[C@H](N2Cc3cc(N4CCN(C[C@H]5CC[C@H](n6cc([C@@H]7c8ccc(O)cc8CC[C@@H]7c7ccccc7)cn6)CC5)CC4)ccc3C2=O)C(=O)N1 ; O=C1CC[C@H](N2Cc3cc(N4CCN(CC5CCN(c6ccc([C@@H]7c8ccc(C(=O)O)cc8CC[C@@H]7c7ccccc7)cc6)CC5)CC4)ccc3C2=O)C(=O)N1] 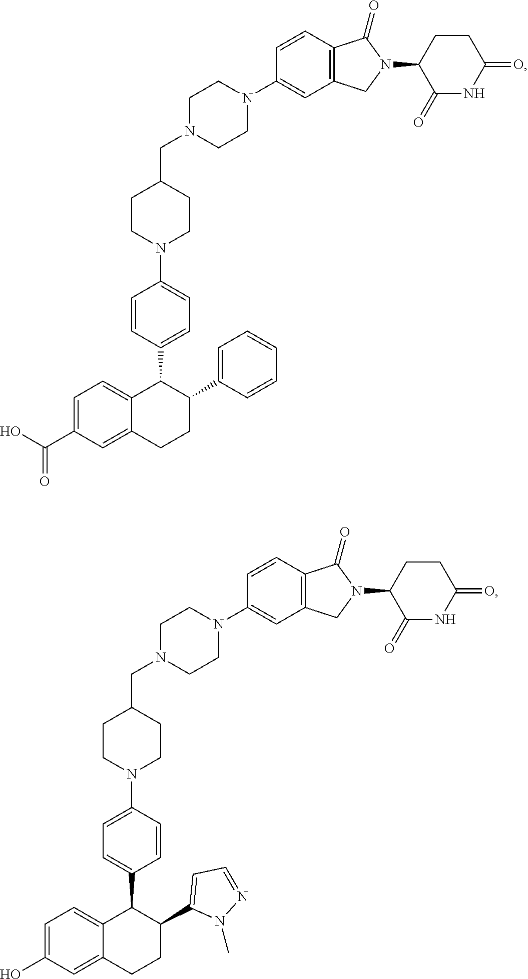 [SMILES: O=C1CC[C@H](N2Cc3cc(N4CCN(CC5CCN(c6ccc([C@H]7c8ccc(C(=O)O)cc8CC[C@H]7c7ccccc7)cc6)CC5)CC4)ccc3C2=O)C(=O)N1 ; Cn1nccc1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] 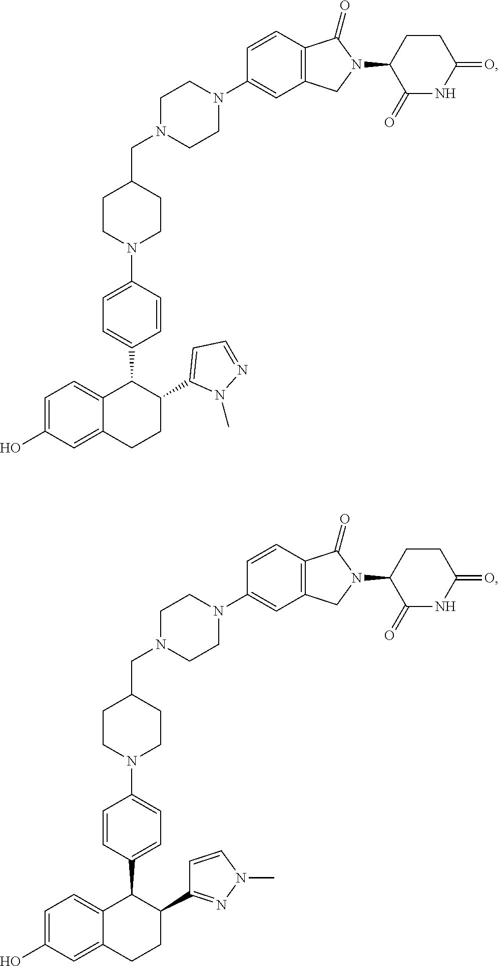 [SMILES: Cn1nccc1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; Cn1ccc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)n1] 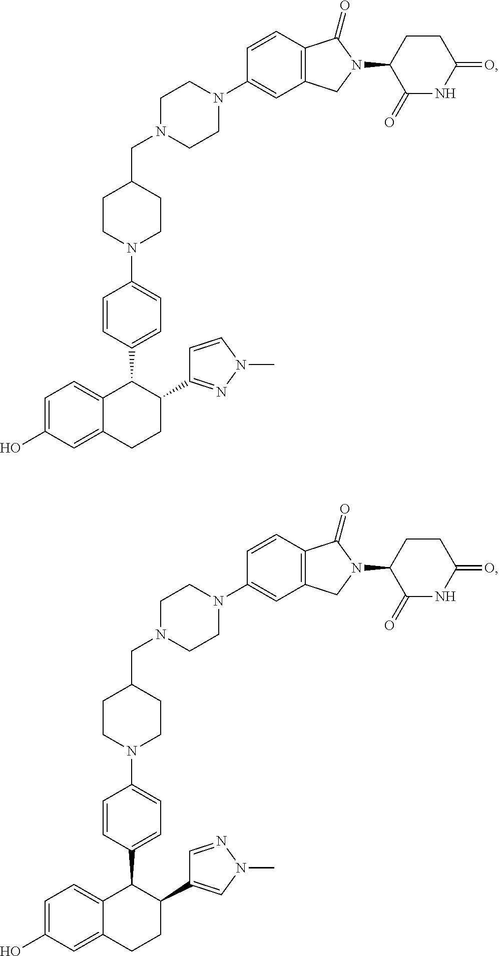 [SMILES: Cn1ccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n1 ; Cn1cc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)cn1] 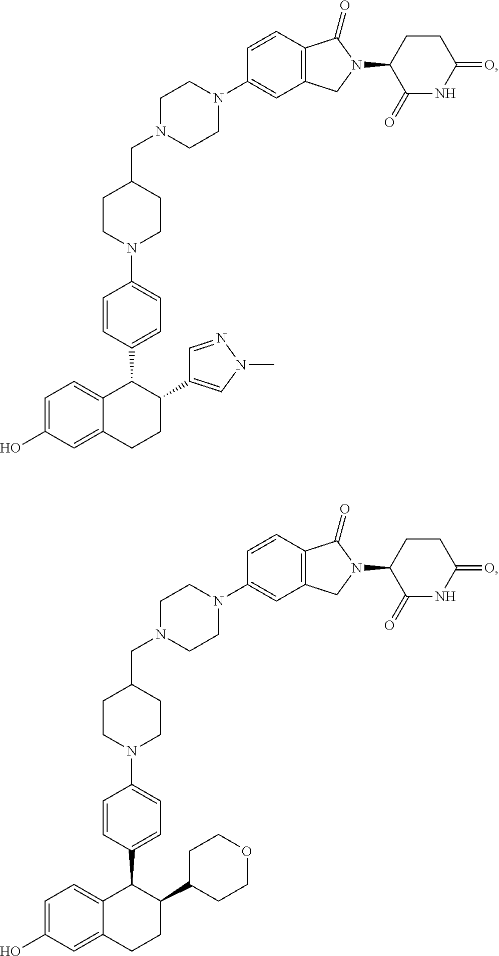 [SMILES: Cn1cc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)cn1 ; O=C1CC[C@H](N2Cc3cc(N4CCN(CC5CCN(c6ccc([C@@H]7c8ccc(O)cc8CC[C@@H]7C7CCOCC7)cc6)CC5)CC4)ccc3C2=O)C(=O)N1] 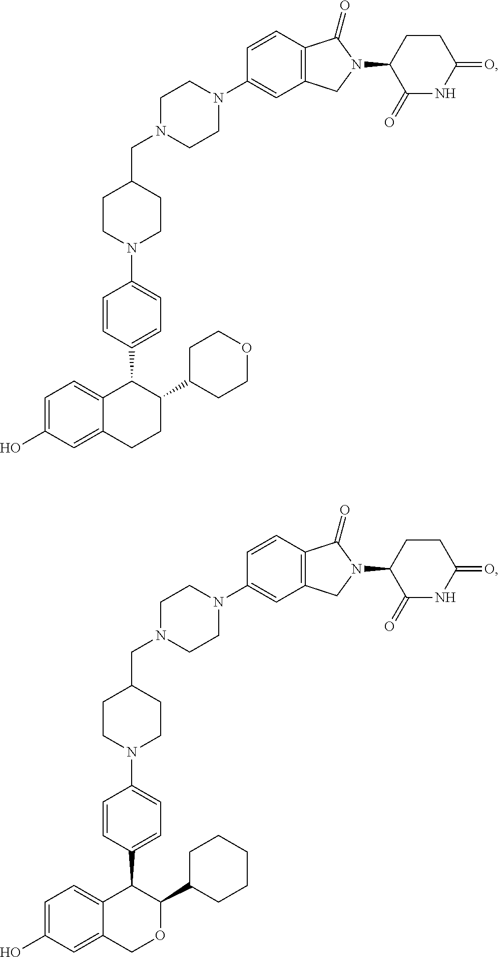 [SMILES: O=C1CC[C@H](N2Cc3cc(N4CCN(CC5CCN(c6ccc([C@H]7c8ccc(O)cc8CC[C@H]7C7CCOCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7C7CCCCC7)cc6)CC5)CC4)ccc3C2=O)C(=O)N1] 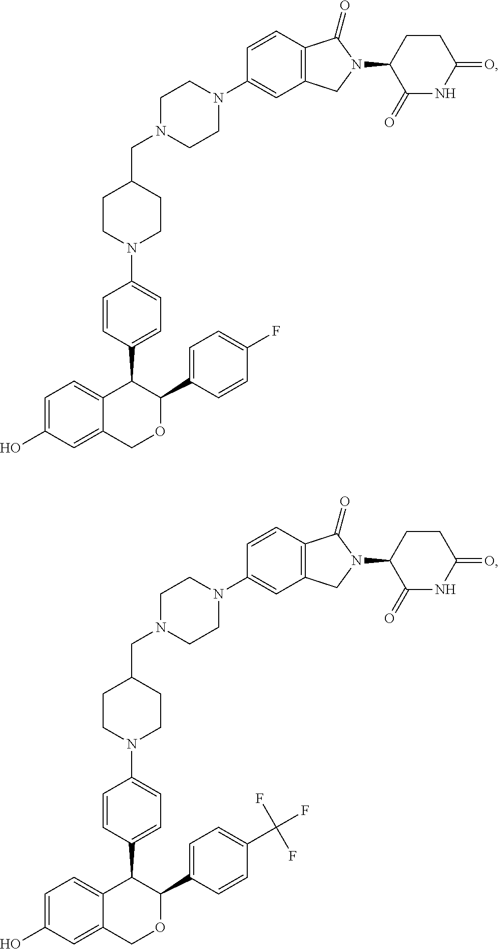 [SMILES: O=C1CC[C@H](N2Cc3cc(N4CCN(CC5CCN(c6ccc([C@@H]7c8ccc(O)cc8CO[C@@H]7c7ccc(F)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccc(C(F)(F)F)cc7)cc6)CC5)CC4)ccc3C2=O)C(=O)N1] 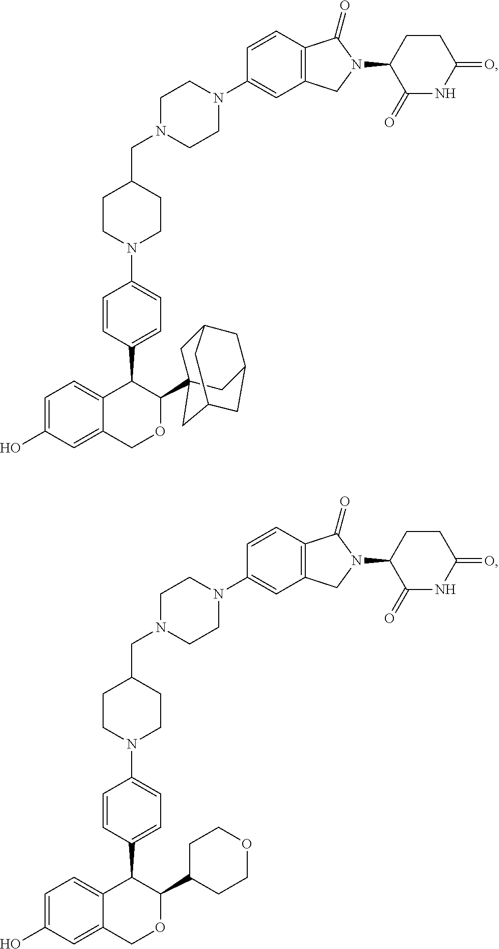 [SMILES: O=C1CC[C@H](N2Cc3cc(N4CCN(CC5CCN(c6ccc([C@@H]7c8ccc(O)cc8CO[C@@H]7C78CC9CC(CC(C9)C7)C8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7C7CCOCC7)cc6)CC5)CC4)ccc3C2=O)C(=O)N1] 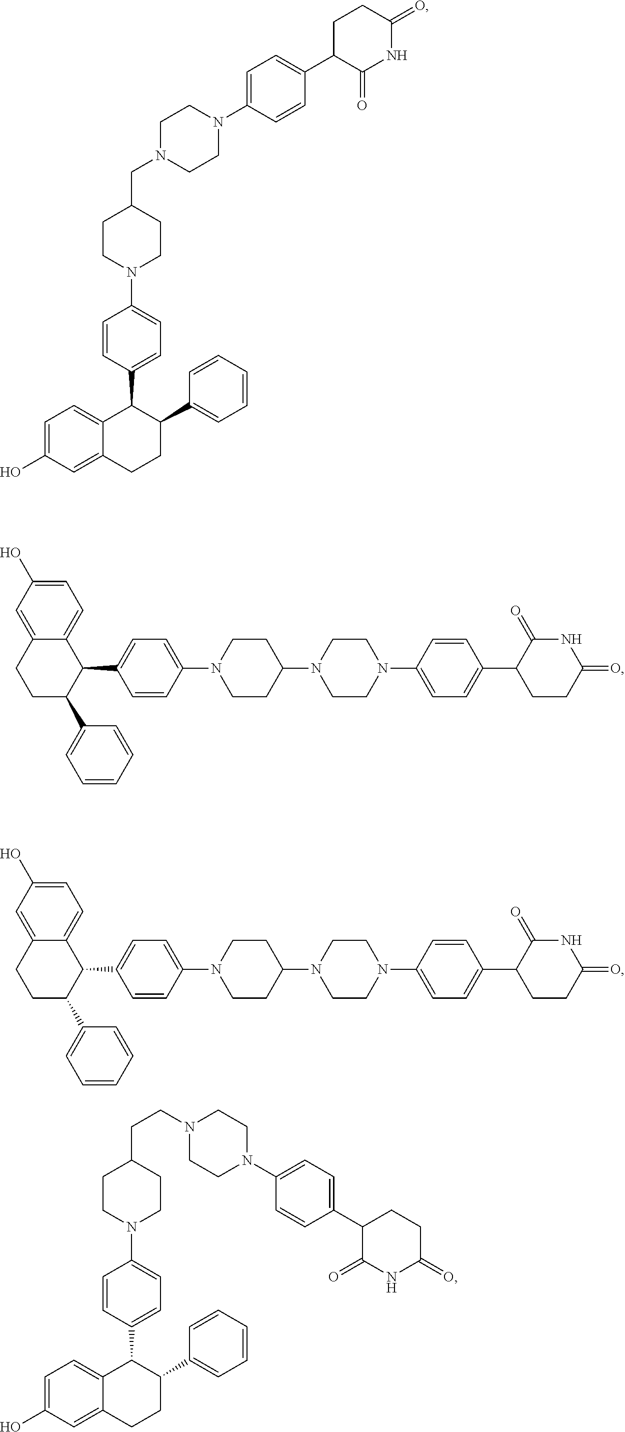 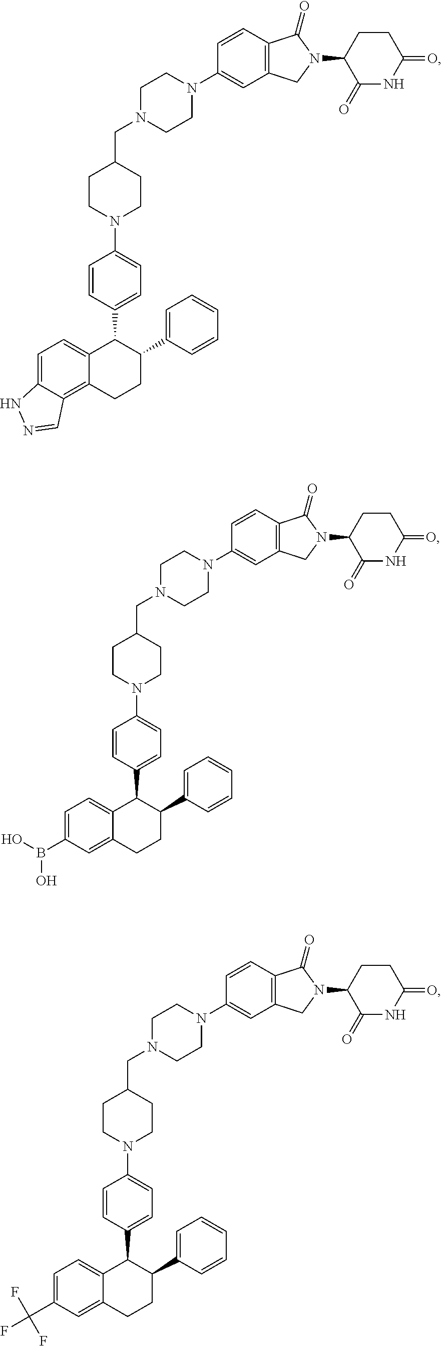 [SMILES: O=C1CC[C@H](N2Cc3cc(N4CCN(CC5CCN(c6ccc([C@H]7c8ccc9[nH]ncc9c8CC[C@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(B(O)O)cc8CC[C@@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(C(F)(F)F)cc8CC[C@@H]7c7ccccc7)cc6)CC5)CC4)ccc3C2=O)C(=O)N1] 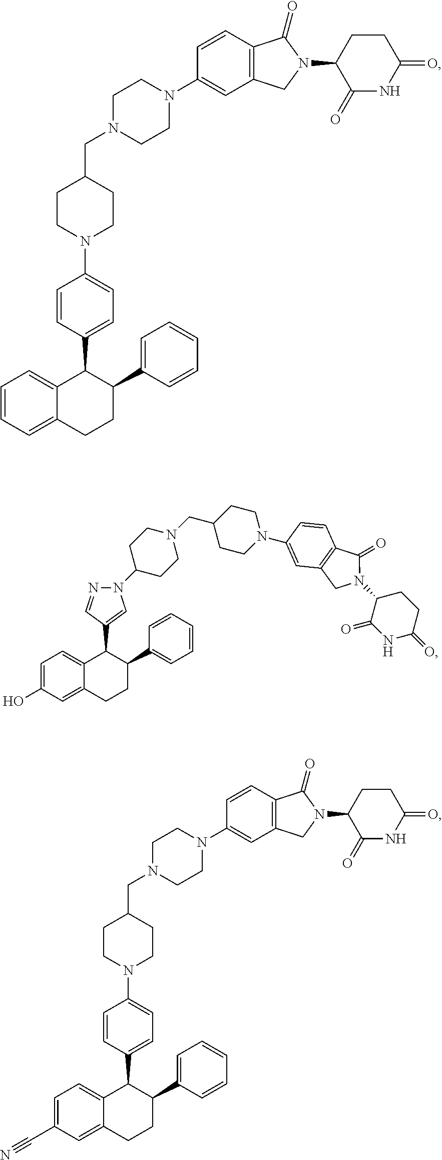 [SMILES: O=C1CC[C@H](N2Cc3cc(N4CCN(CC5CCN(c6ccc([C@@H]7c8ccccc8CC[C@@H]7c7ccccc7)cc6)CC5)CC4)ccc3C2=O)C(=O)N1 ; O=C1CC[C@@H](N2Cc3cc(N4CCC(CN5CCC(n6cc([C@@H]7c8ccc(O)cc8CC[C@@H]7c7ccccc7)cn6)CC5)CC4)ccc3C2=O)C(=O)N1 ; N#Cc1ccc2c(c1)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1] 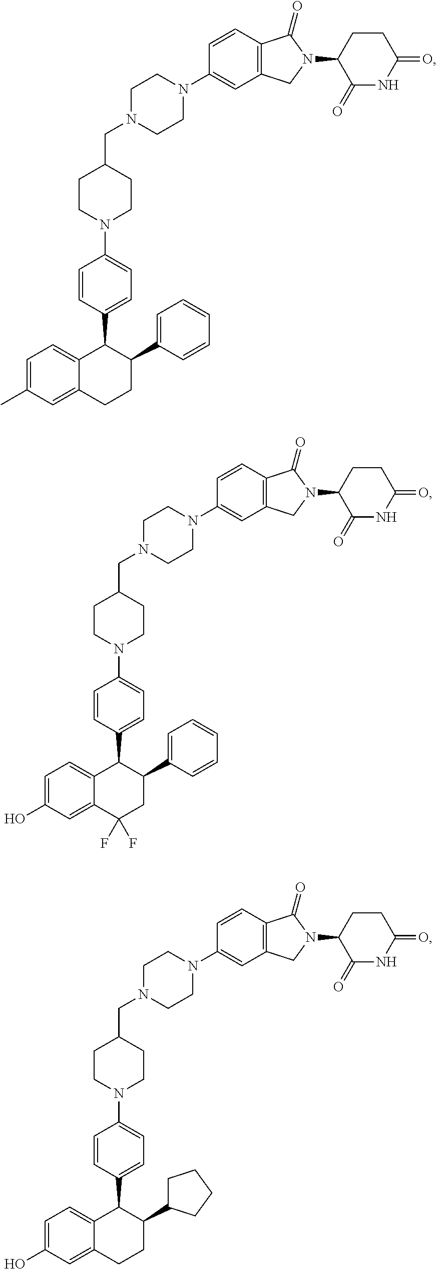 [SMILES: Cc1ccc2c(c1)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCCC7)cc6)CC5)CC4)ccc3C2=O)C(=O)N1] 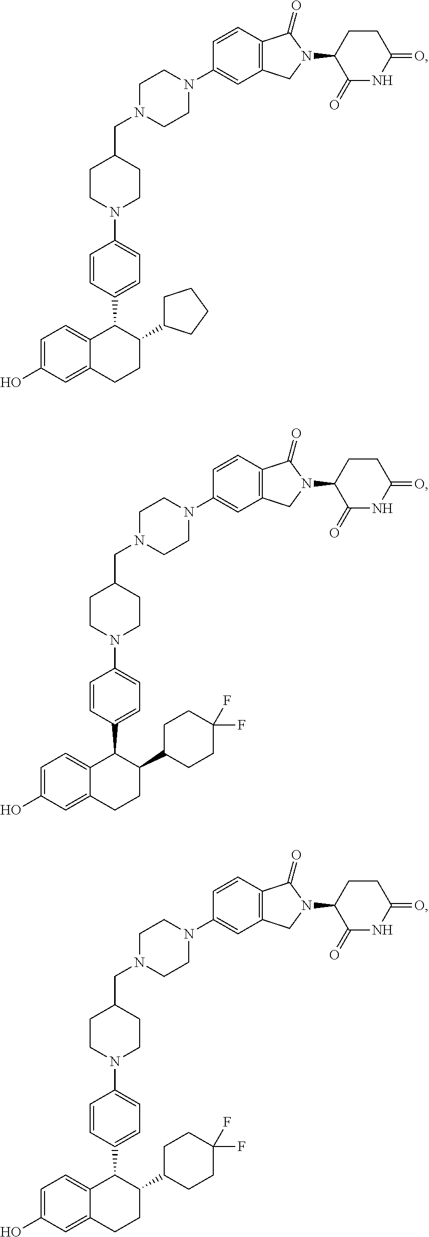 [SMILES: O=C1CC[C@H](N2Cc3cc(N4CCN(CC5CCN(c6ccc([C@H]7c8ccc(O)cc8CC[C@H]7C7CCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCC(F)(F)CC7)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7C7CCC(F)(F)CC7)cc6)CC5)CC4)ccc3C2=O)C(=O)N1] 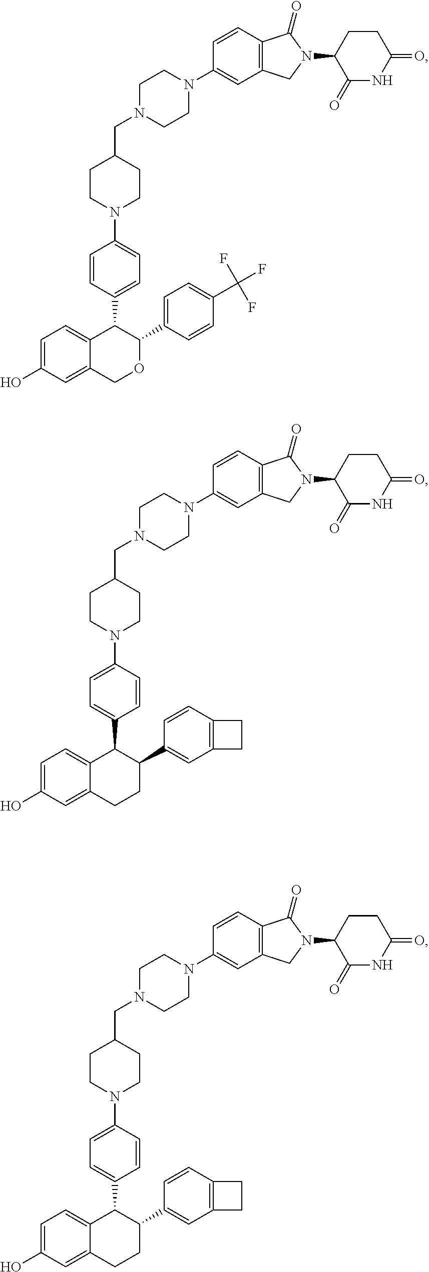 [SMILES: O=C1CC[C@H](N2Cc3cc(N4CCN(CC5CCN(c6ccc([C@H]7c8ccc(O)cc8CO[C@H]7c7ccc(C(F)(F)F)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccc8c(c7)CC8)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc8c(c7)CC8)cc6)CC5)CC4)ccc3C2=O)C(=O)N1] 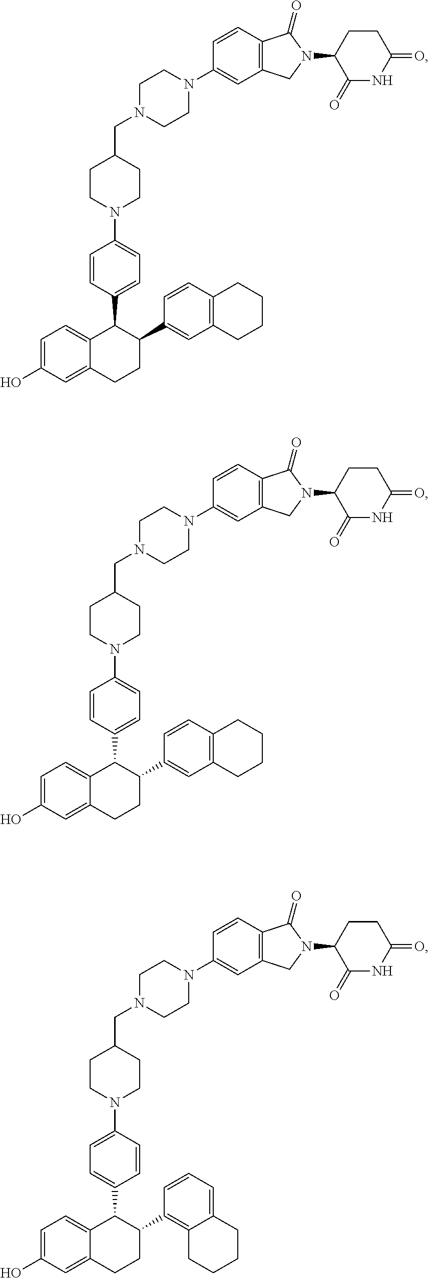 [SMILES: O=C1CC[C@H](N2Cc3cc(N4CCN(CC5CCN(c6ccc([C@@H]7c8ccc(O)cc8CC[C@@H]7c7ccc8c(c7)CCCC8)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc8c(c7)CCCC8)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7cccc8c7CCCC8)cc6)CC5)CC4)ccc3C2=O)C(=O)N1] 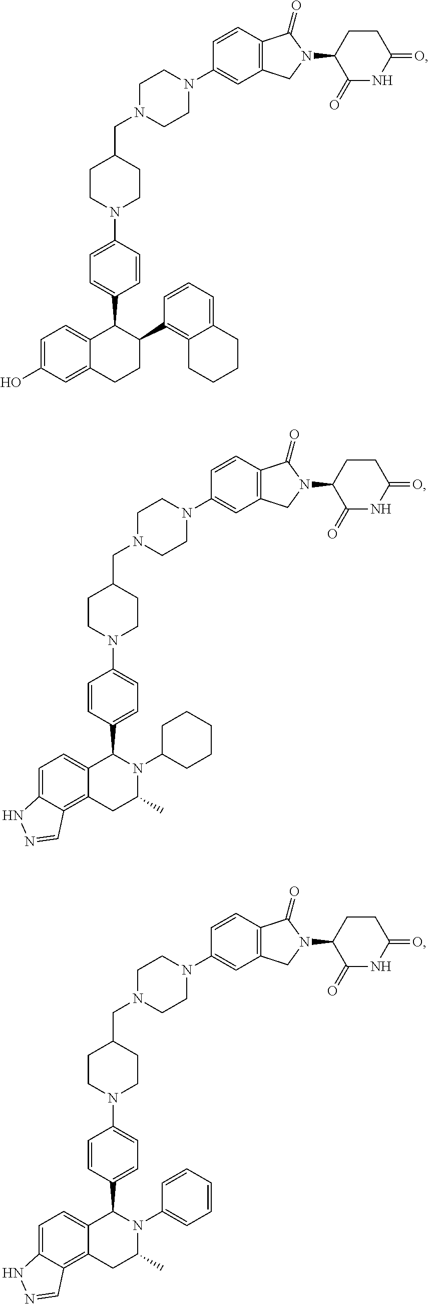 [SMILES: O=C1CC[C@H](N2Cc3cc(N4CCN(CC5CCN(c6ccc([C@@H]7c8ccc(O)cc8CC[C@@H]7c7cccc8c7CCCC8)cc6)CC5)CC4)ccc3C2=O)C(=O)N1 ; C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1C1CCCCC1 ; C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1c1ccccc1] 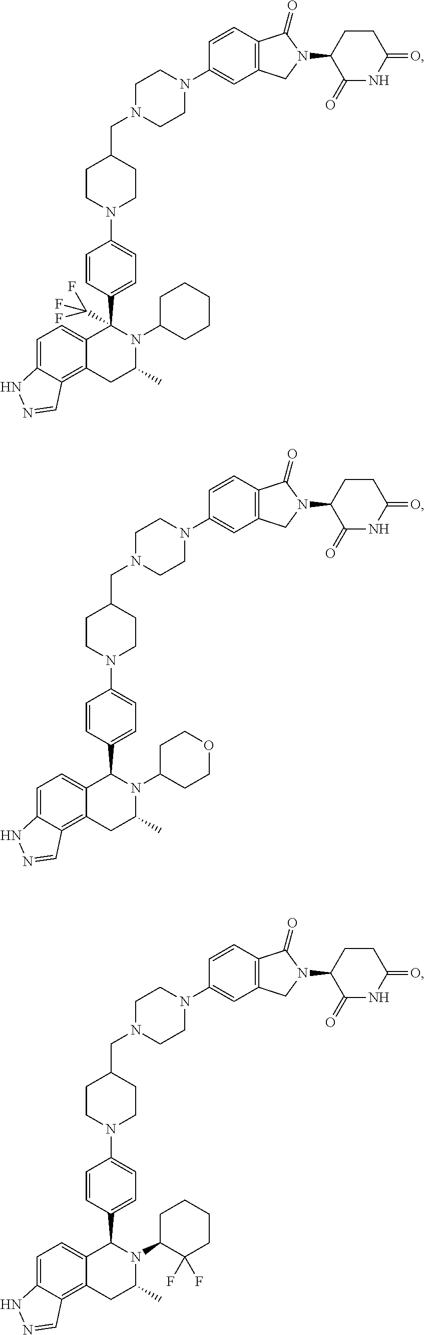 [SMILES: C[C@@H]1Cc2c(ccc3[nH]ncc23)[C@](c2ccc(N3CCC(CN4CCN(c5ccc6c(c5)CN([C@H]5CCC(=O)NC5=O)C6=O)CC4)CC3)cc2)(C(F)(F)F)N1C1CCCCC1 ; C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1C1CCOCC1 ; C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1[C@H]1CCCCC1(F)F] 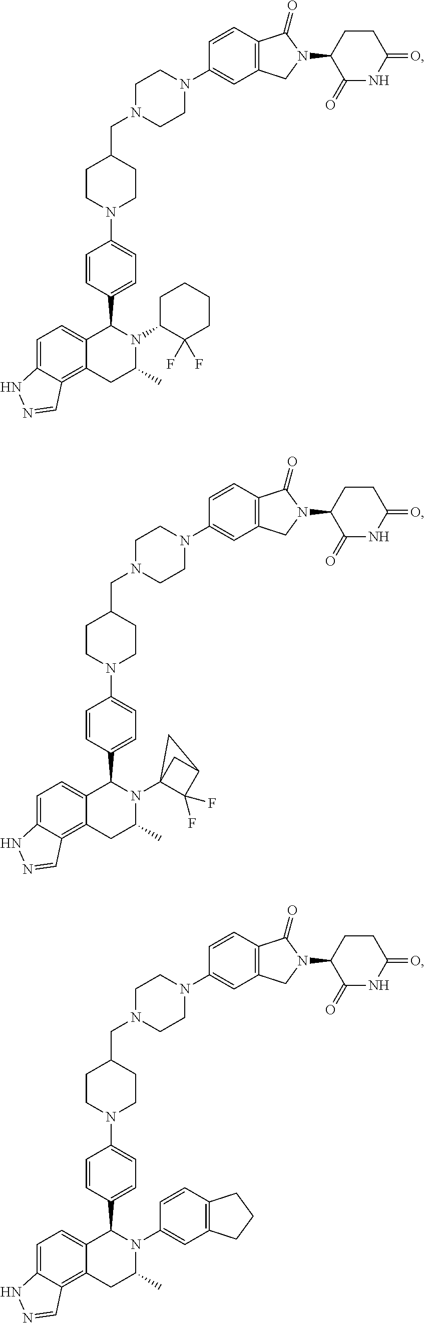 [SMILES: C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1[C@@H]1CCCCC1(F)F ; C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1C12CC(C1)C2(F)F ; C[C@@H]1Cc2c(ccc3[nH]ncc23)[C@@H](c2ccc(N3CCC(CN4CCN(c5ccc6c(c5)CN([C@H]5CCC(=O)NC5=O)C6=O)CC4)CC3)cc2)N1c1ccc2c(c1)CCC2] 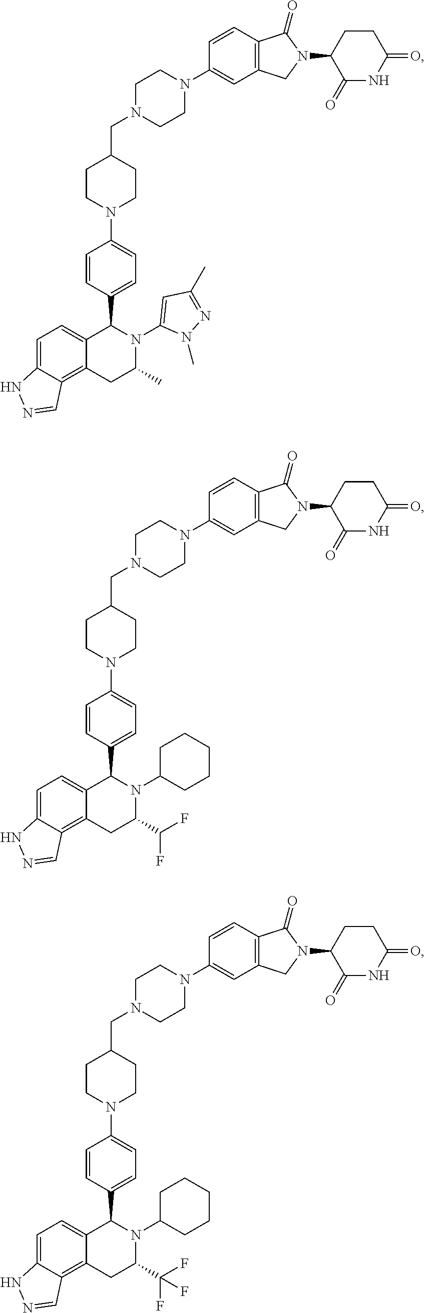 [SMILES: Cc1cc(N2[C@H](c3ccc(N4CCC(CN5CCN(c6ccc7c(c6)CN([C@H]6CCC(=O)NC6=O)C7=O)CC5)CC4)cc3)c3ccc4[nH]ncc4c3C[C@H]2C)n(C)n1 ; O=C1CC[C@H](N2Cc3cc(N4CCN(CC5CCN(c6ccc([C@@H]7c8ccc9[nH]ncc9c8C[C@@H](C(F)F)N7C7CCCCC7)cc6)CC5)CC4)ccc3C2=O)C(=O)N1 ; O=C1CC[C@H](N2Cc3cc(N4CCN(CC5CCN(c6ccc([C@@H]7c8ccc9[nH]ncc9c8C[C@@H](C(F)(F)F)N7C7CCCCC7)cc6)CC5)CC4)ccc3C2=O)C(=O)N1] 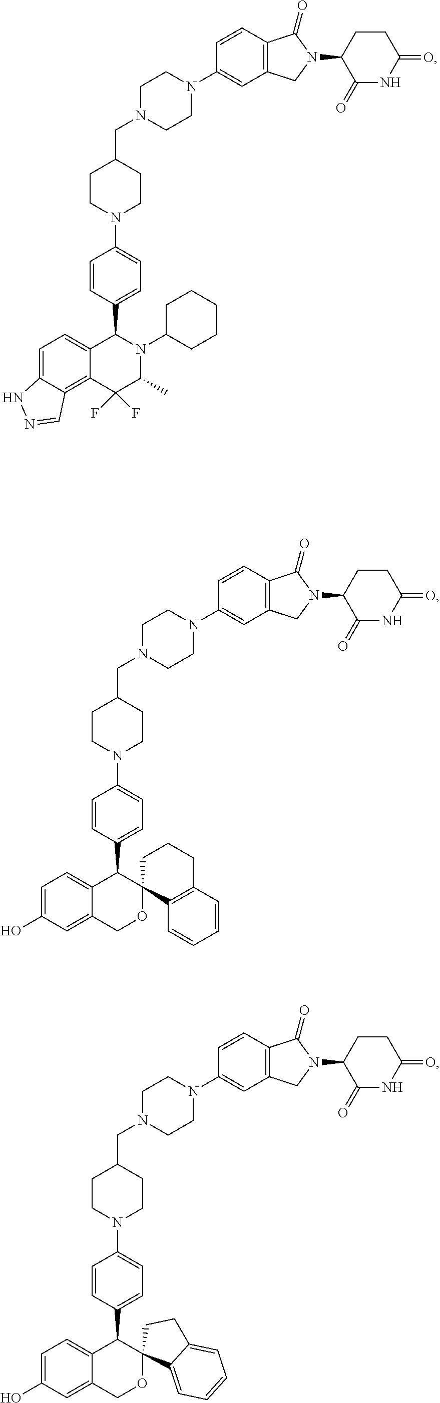 [SMILES: C[C@H]1N(C2CCCCC2)[C@H](c2ccc(N3CCC(CN4CCN(c5ccc6c(c5)CN([C@H]5CCC(=O)NC5=O)C6=O)CC4)CC3)cc2)c2ccc3[nH]ncc3c2C1(F)F ; O=C1CC[C@H](N2Cc3cc(N4CCN(CC5CCN(c6ccc([C@@H]7c8ccc(O)cc8CO[C@@]78CCCc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@]78CCc7ccccc78)cc6)CC5)CC4)ccc3C2=O)C(=O)N1] 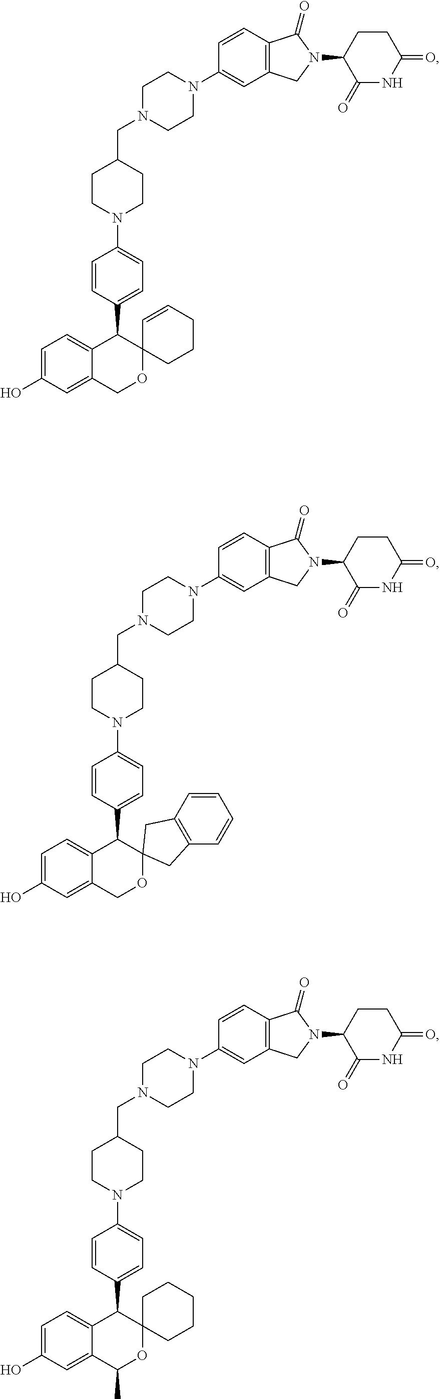 [SMILES: O=C1CC[C@H](N2Cc3cc(N4CCN(CC5CCN(c6ccc([C@@H]7c8ccc(O)cc8COC78C=CCCC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8COC78Cc7ccccc7C8)cc6)CC5)CC4)ccc3C2=O)C(=O)N1 ; C[C@@H]1OC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] 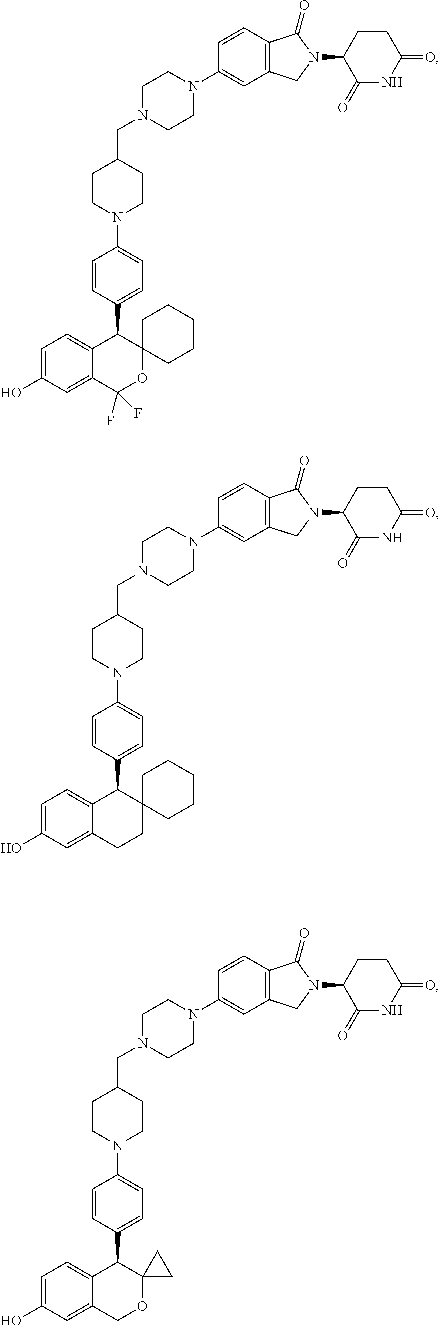 [SMILES: O=C1CC[C@H](N2Cc3cc(N4CCN(CC5CCN(c6ccc([C@@H]7c8ccc(O)cc8C(F)(F)OC78CCCCC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CCC78CCCCC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8COC78CC8)cc6)CC5)CC4)ccc3C2=O)C(=O)N1] 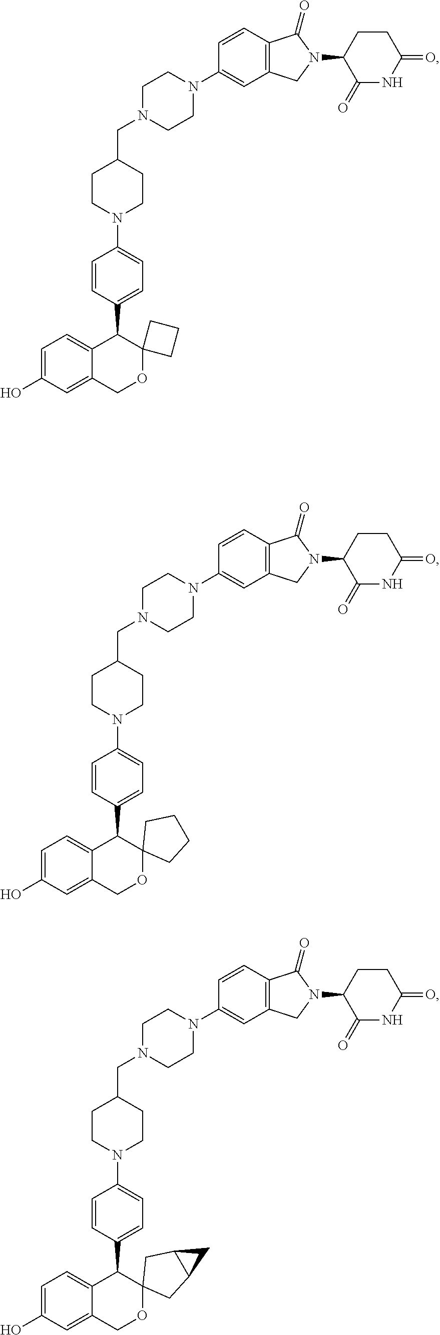 [SMILES: O=C1CC[C@H](N2Cc3cc(N4CCN(CC5CCN(c6ccc([C@@H]7c8ccc(O)cc8COC78CCC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8COC78CCCC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8COC78C[C@H]7C[C@H]7C8)cc6)CC5)CC4)ccc3C2=O)C(=O)N1] 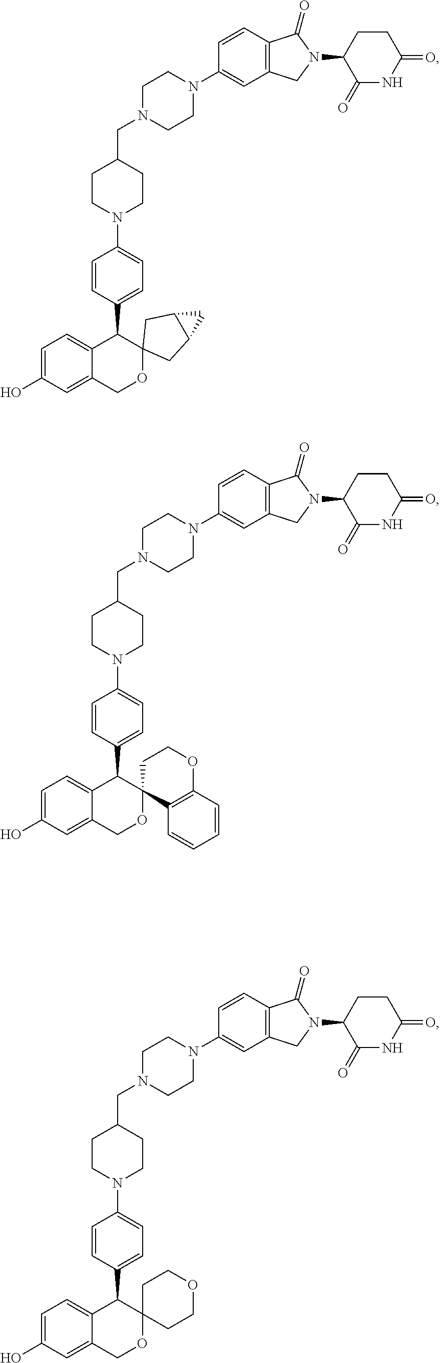 [SMILES: O=C1CC[C@H](N2Cc3cc(N4CCN(CC5CCN(c6ccc([C@@H]7c8ccc(O)cc8COC78C[C@H]7C[C@H]7C8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@]78CCOc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8COC78CCOCC8)cc6)CC5)CC4)ccc3C2=O)C(=O)N1] 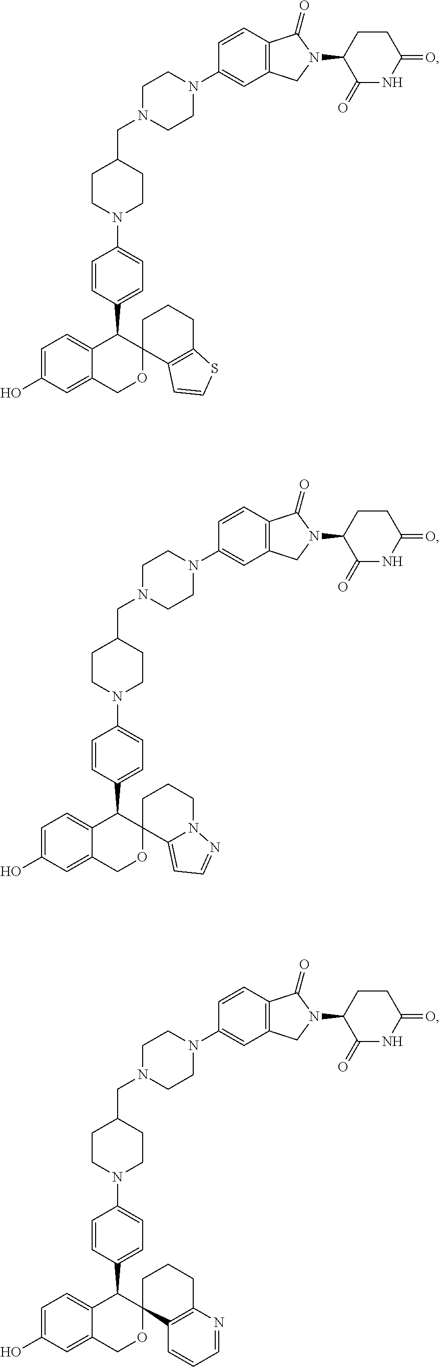 [SMILES: O=C1CC[C@H](N2Cc3cc(N4CCN(CC5CCN(c6ccc([C@@H]7c8ccc(O)cc8COC78CCCc7sccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8COC78CCCn7nccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@]78CCCc7ncccc78)cc6)CC5)CC4)ccc3C2=O)C(=O)N1] 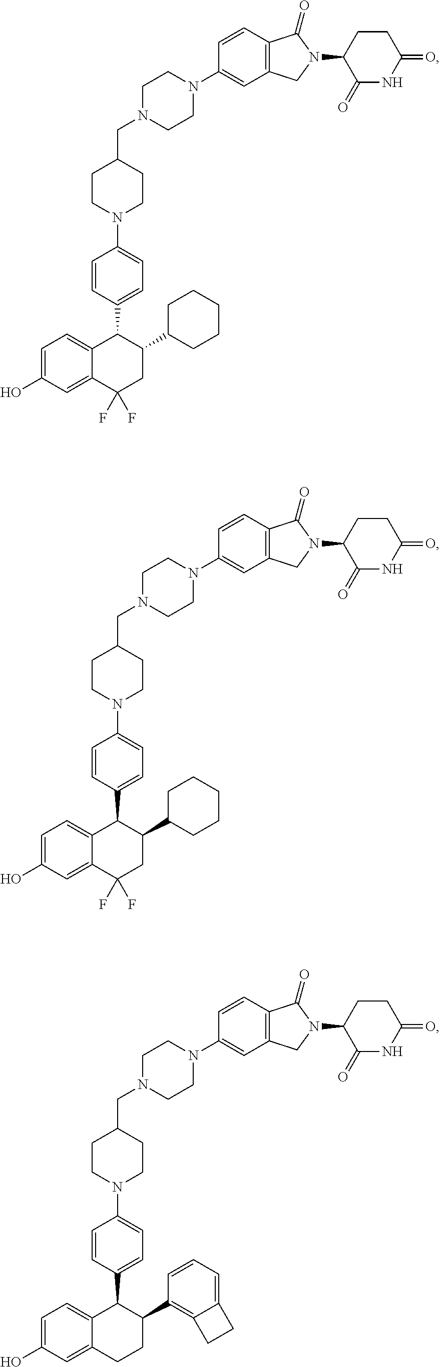 [SMILES: O=C1CC[C@H](N2Cc3cc(N4CCN(CC5CCN(c6ccc([C@H]7c8ccc(O)cc8C(F)(F)C[C@H]7C7CCCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7C7CCCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc8c7CC8)cc6)CC5)CC4)ccc3C2=O)C(=O)N1] 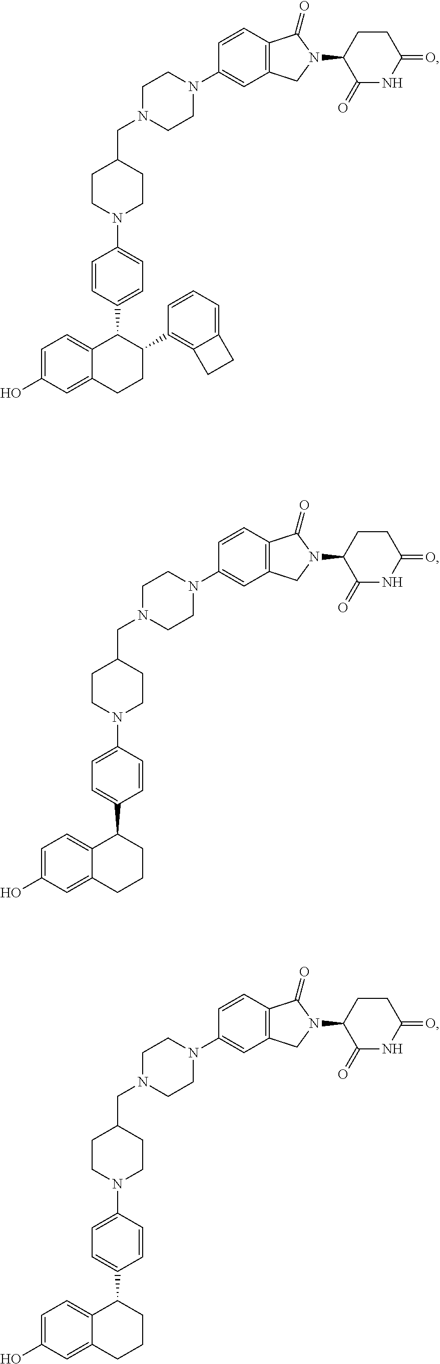 [SMILES: O=C1CC[C@H](N2Cc3cc(N4CCN(CC5CCN(c6ccc([C@H]7c8ccc(O)cc8CC[C@H]7c7cccc8c7CC8)cc6)CC5)CC4)ccc3C2=O)C(=O)N1 ; O=C1CC[C@H](N2Cc3cc(N4CCN(CC5CCN(c6ccc([C@H]7CCCc8cc(O)ccc87)cc6)CC5)CC4)ccc3C2=O)C(=O)N1 ; O=C1CC[C@H](N2Cc3cc(N4CCN(CC5CCN(c6ccc([C@@H]7CCCc8cc(O)ccc87)cc6)CC5)CC4)ccc3C2=O)C(=O)N1] 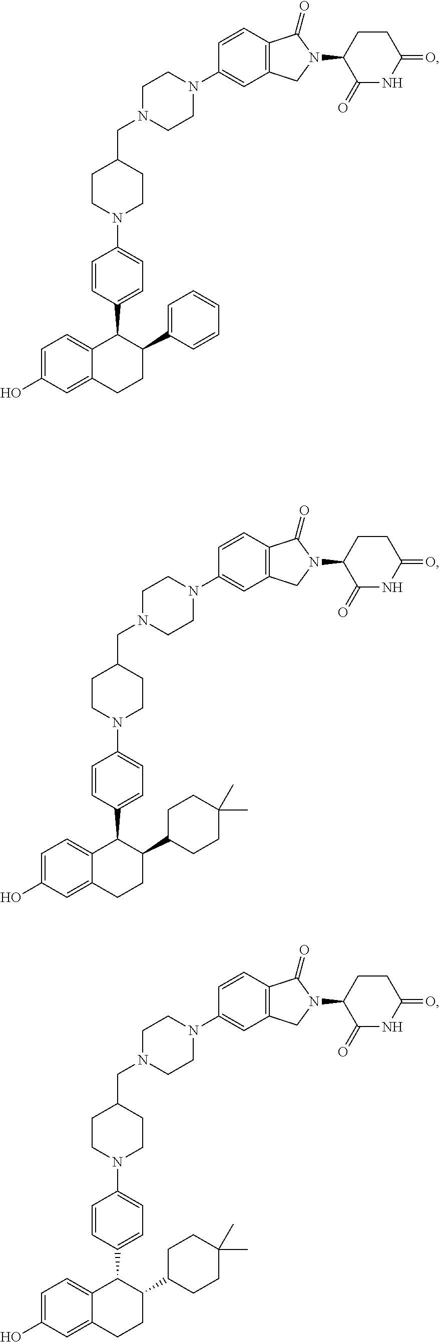 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1 ; CC1(C)CCC([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)CC1 ; CC1(C)CCC([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)CC1] 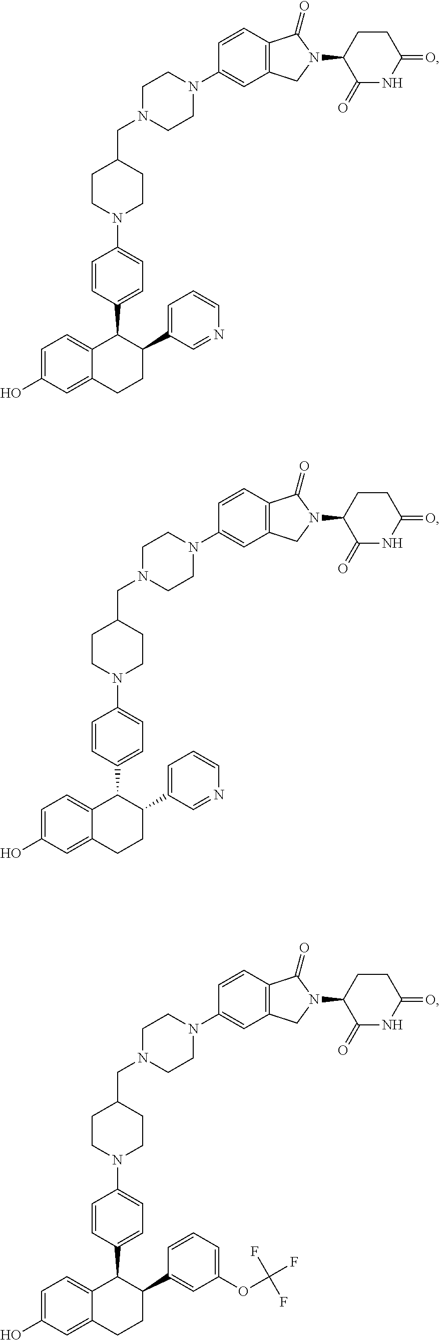 [SMILES: O=C1CC[C@H](N2Cc3cc(N4CCN(CC5CCN(c6ccc([C@@H]7c8ccc(O)cc8CC[C@@H]7c7cccnc7)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7cccnc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc(OC(F)(F)F)c7)cc6)CC5)CC4)ccc3C2=O)C(=O)N1] 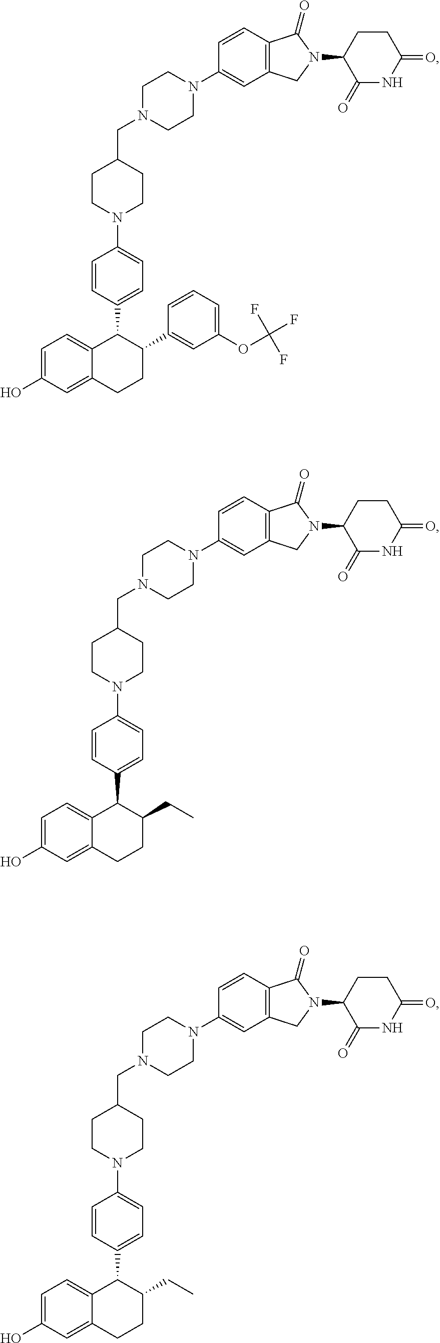 [SMILES: O=C1CC[C@H](N2Cc3cc(N4CCN(CC5CCN(c6ccc([C@H]7c8ccc(O)cc8CC[C@H]7c7cccc(OC(F)(F)F)c7)cc6)CC5)CC4)ccc3C2=O)C(=O)N1 ; CC[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; CC[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] 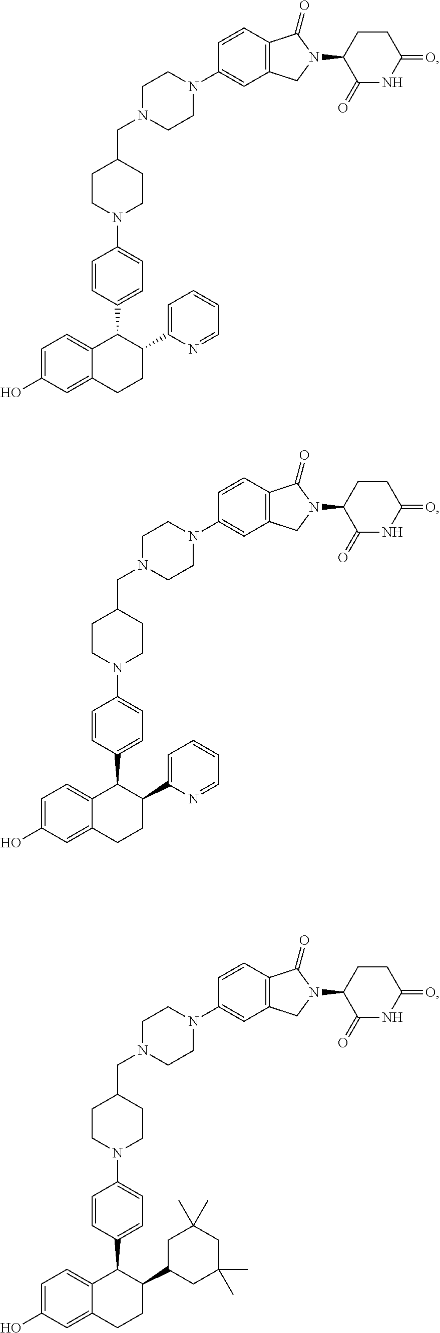 [SMILES: O=C1CC[C@H](N2Cc3cc(N4CCN(CC5CCN(c6ccc([C@H]7c8ccc(O)cc8CC[C@H]7c7ccccn7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccccn7)cc6)CC5)CC4)ccc3C2=O)C(=O)N1 ; CC1(C)CC([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)CC(C)(C)C1] 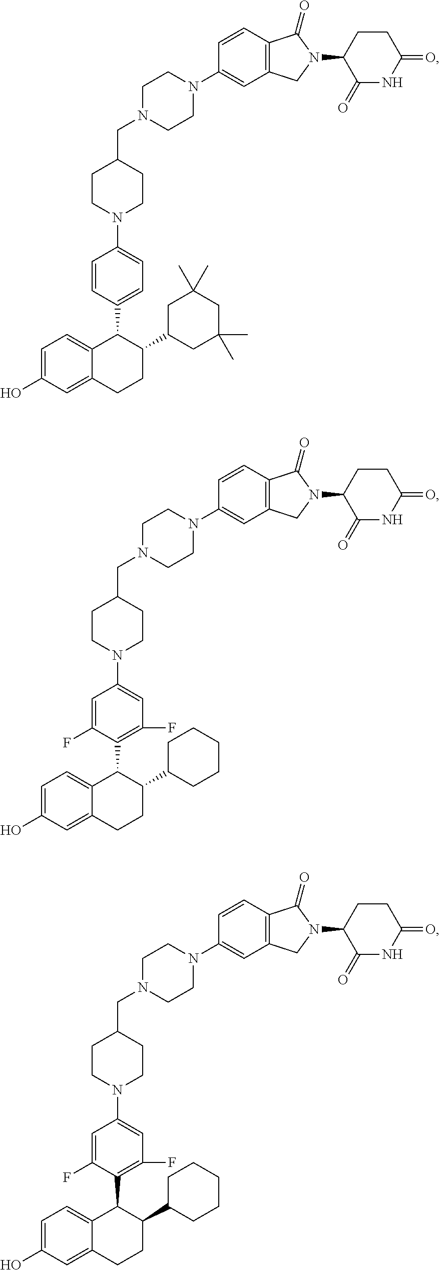 [SMILES: CC1(C)CC([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)CC(C)(C)C1 ; O=C1CC[C@H](N2Cc3cc(N4CCN(CC5CCN(c6cc(F)c([C@H]7c8ccc(O)cc8CC[C@H]7C7CCCCC7)c(F)c6)CC5)CC4)ccc3C2=O)C(=O)N1 ; O=C1CC[C@H](N2Cc3cc(N4CCN(CC5CCN(c6cc(F)c([C@@H]7c8ccc(O)cc8CC[C@@H]7C7CCCCC7)c(F)c6)CC5)CC4)ccc3C2=O)C(=O)N1] 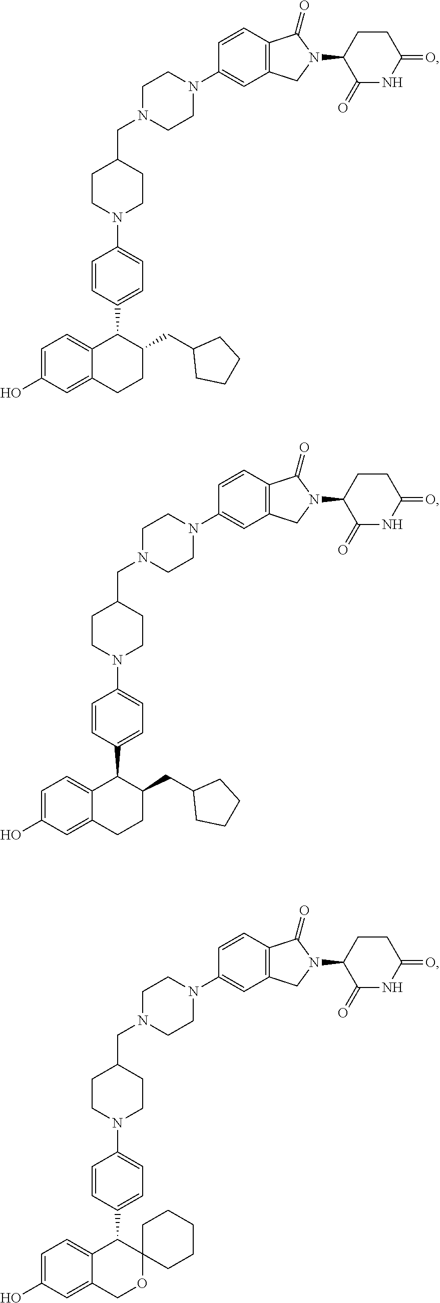 [SMILES: O=C1CC[C@H](N2Cc3cc(N4CCN(CC5CCN(c6ccc([C@H]7c8ccc(O)cc8CC[C@H]7CC7CCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7CC7CCCC7)cc6)CC5)CC4)ccc3C2=O)C(=O)N1 ; O=C1CC[C@H](N2Cc3cc(N4CCN(CC5CCN(c6ccc([C@H]7c8ccc(O)cc8COC78CCCCC8)cc6)CC5)CC4)ccc3C2=O)C(=O)N1] 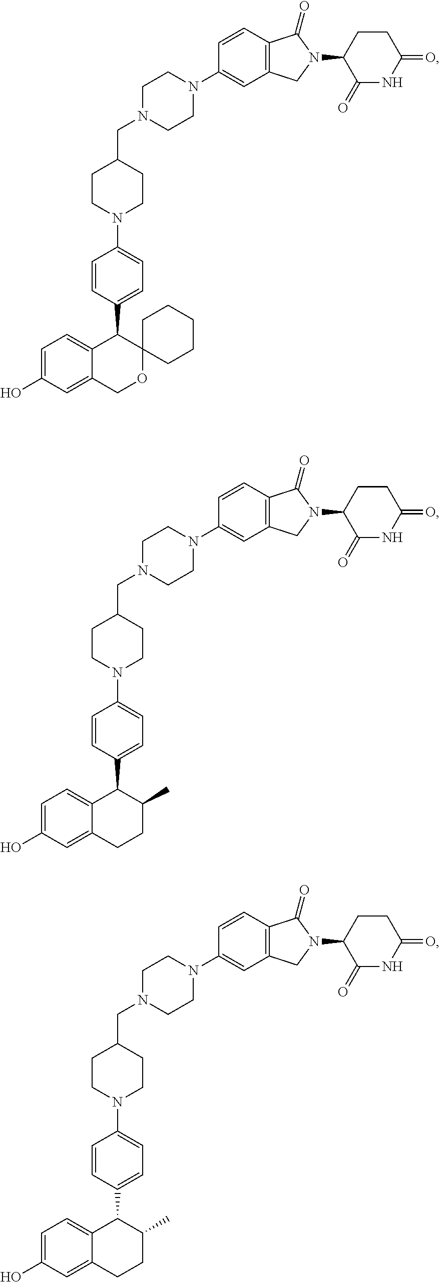 [SMILES: O=C1CC[C@H](N2Cc3cc(N4CCN(CC5CCN(c6ccc([C@@H]7c8ccc(O)cc8COC78CCCCC8)cc6)CC5)CC4)ccc3C2=O)C(=O)N1 ; C[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; C[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] 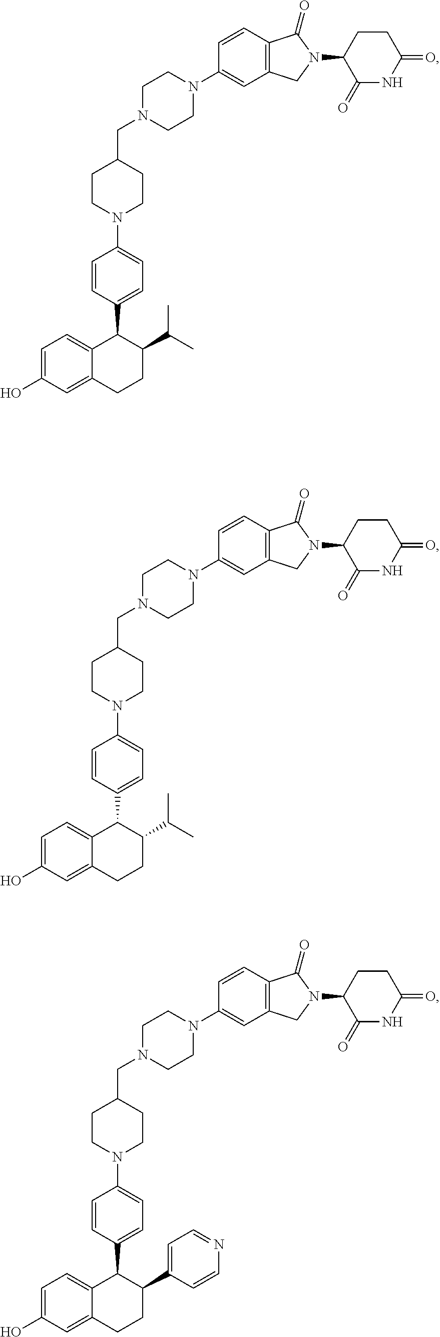 [SMILES: CC(C)[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; CC(C)[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8CC[C@@H]7c7ccncc7)cc6)CC5)CC4)ccc3C2=O)C(=O)N1] 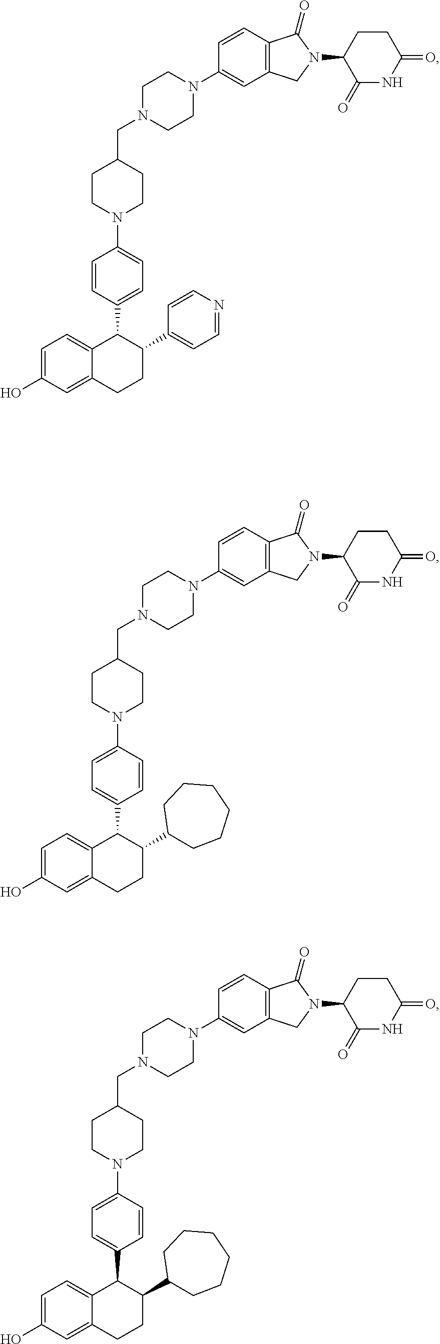 [SMILES: O=C1CC[C@H](N2Cc3cc(N4CCN(CC5CCN(c6ccc([C@H]7c8ccc(O)cc8CC[C@H]7c7ccncc7)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7C7CCCCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCCCCC7)cc6)CC5)CC4)ccc3C2=O)C(=O)N1] 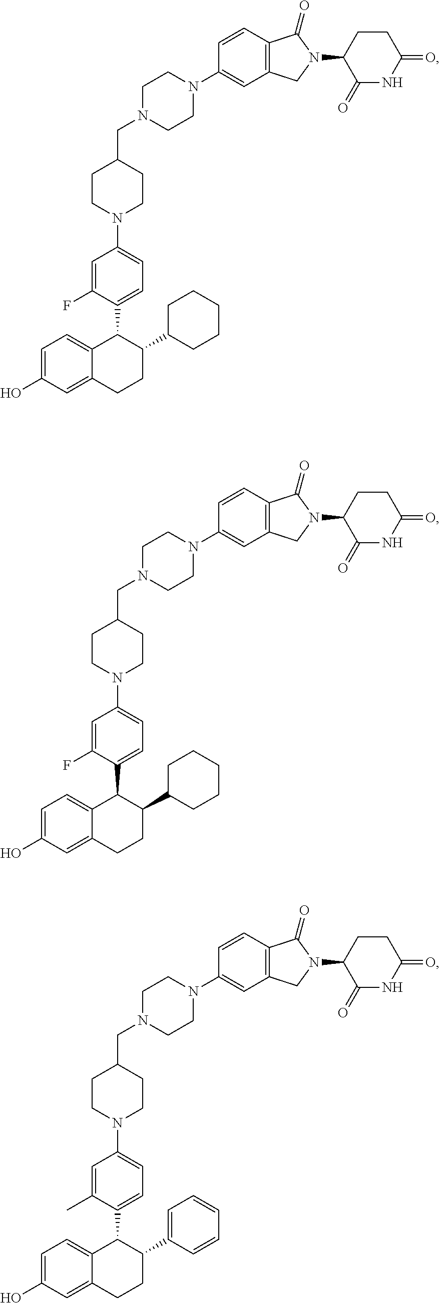 [SMILES: O=C1CC[C@H](N2Cc3cc(N4CCN(CC5CCN(c6ccc([C@H]7c8ccc(O)cc8CC[C@H]7C7CCCCC7)c(F)c6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCCCC7)c(F)c6)CC5)CC4)ccc3C2=O)C(=O)N1 ; Cc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1c1ccccc1] 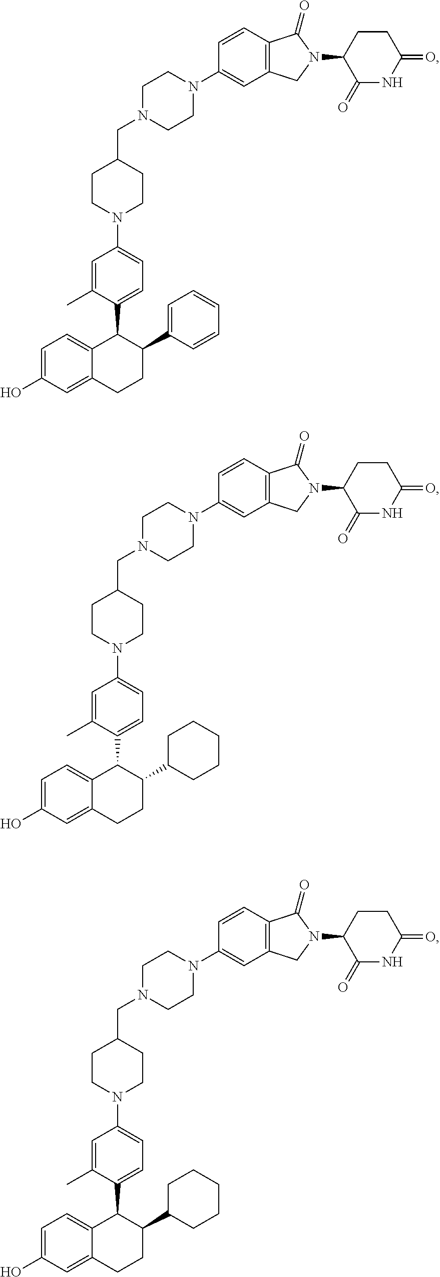 [SMILES: Cc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1c1ccccc1 ; Cc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1C1CCCCC1 ; Cc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1C1CCCCC1] 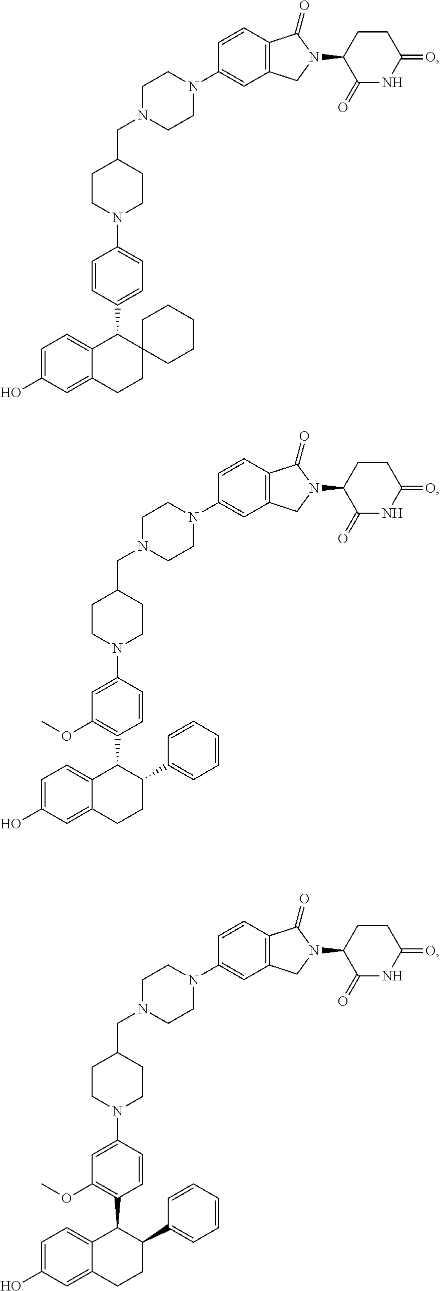 [SMILES: O=C1CC[C@H](N2Cc3cc(N4CCN(CC5CCN(c6ccc([C@H]7c8ccc(O)cc8CCC78CCCCC8)cc6)CC5)CC4)ccc3C2=O)C(=O)N1 ; COc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1c1ccccc1 ; COc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1c1ccccc1] 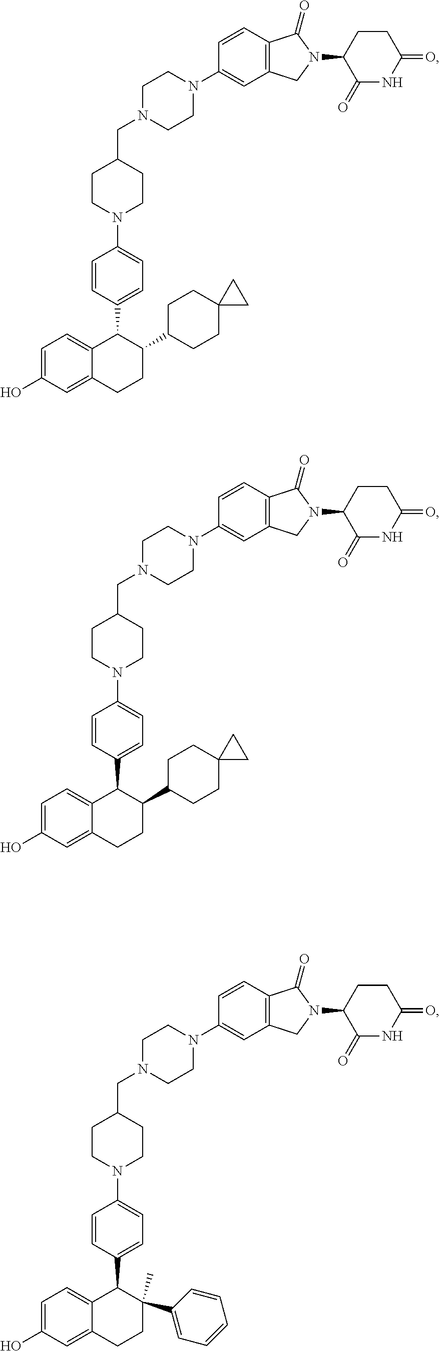 [SMILES: O=C1CC[C@H](N2Cc3cc(N4CCN(CC5CCN(c6ccc([C@H]7c8ccc(O)cc8CC[C@H]7C7CCC8(CC7)CC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCC8(CC7)CC8)cc6)CC5)CC4)ccc3C2=O)C(=O)N1 ; C[C@]1(c2ccccc2)CCc2cc(O)ccc2[C@H]1c1ccc(N2CCC(CN3CCN(c4ccc5c(c4)CN([C@H]4CCC(=O)NC4=O)C5=O)CC3)CC2)cc1] 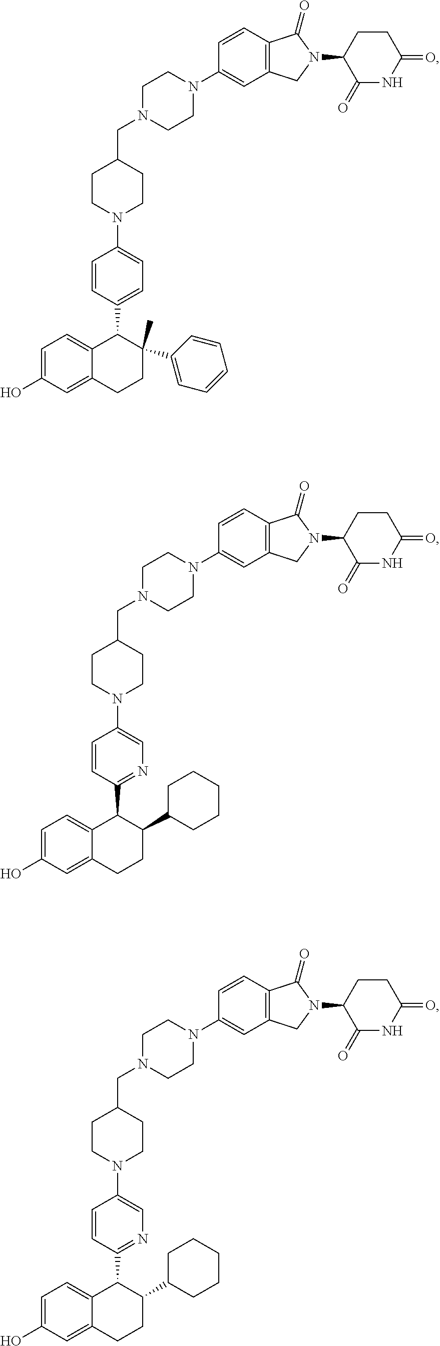 [SMILES: C[C@@]1(c2ccccc2)CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8CC[C@@H]7C7CCCCC7)nc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7C7CCCCC7)nc6)CC5)CC4)ccc3C2=O)C(=O)N1] 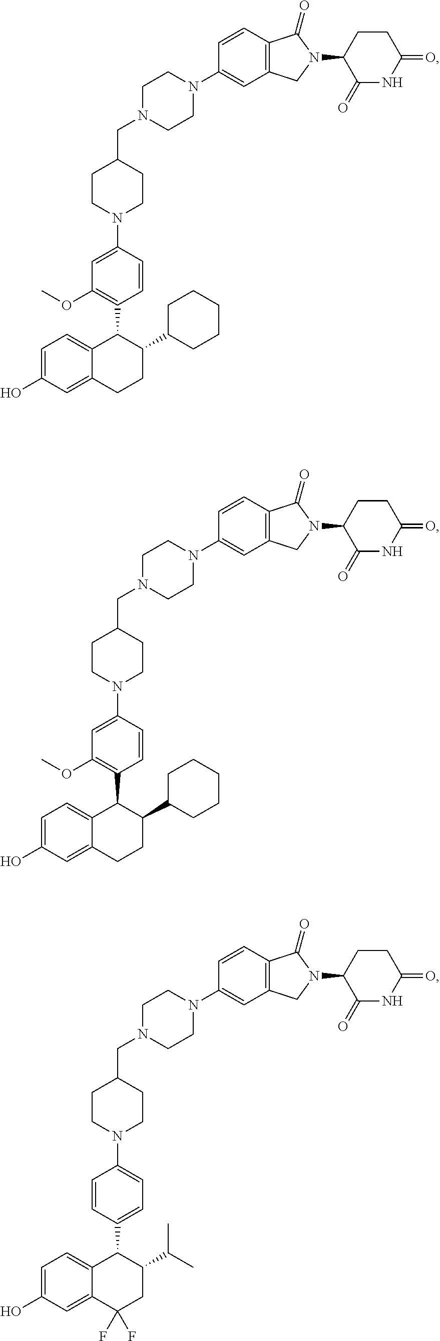 [SMILES: COc1cc(N2CCC(CN3CCN(c4ccc5c(c4)CN([C@H]4CCC(=O)NC4=O)C5=O)CC3)CC2)ccc1[C@H]1c2ccc(O)cc2CC[C@H]1C1CCCCC1 ; COc1cc(N2CCC(CN3CCN(c4ccc5c(c4)CN([C@H]4CCC(=O)NC4=O)C5=O)CC3)CC2)ccc1[C@@H]1c2ccc(O)cc2CC[C@@H]1C1CCCCC1 ; CC(C)[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1] 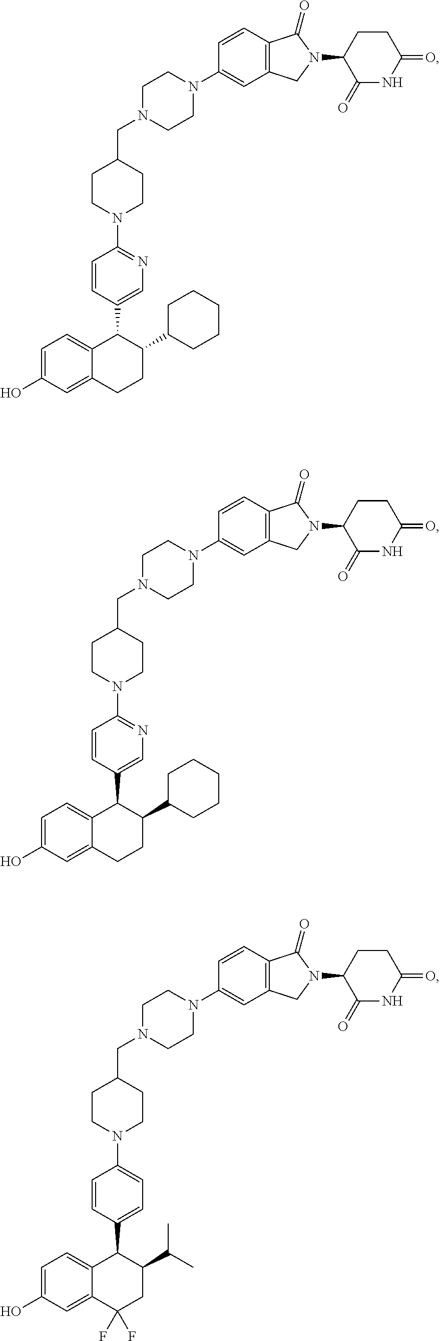 [SMILES: O=C1CC[C@H](N2Cc3cc(N4CCN(CC5CCN(c6ccc([C@H]7c8ccc(O)cc8CC[C@H]7C7CCCCC7)cn6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCCCC7)cn6)CC5)CC4)ccc3C2=O)C(=O)N1 ; CC(C)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] 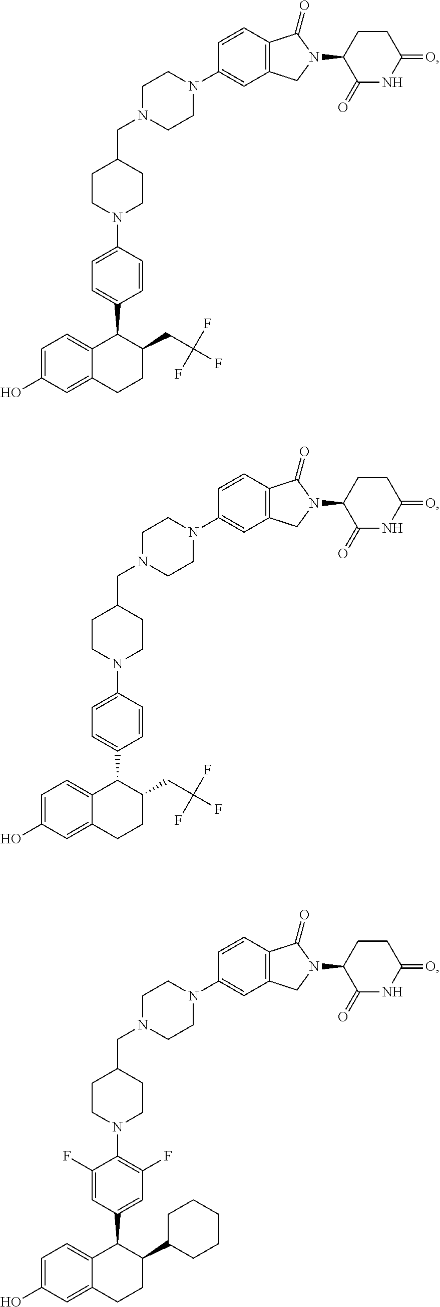 [SMILES: O=C1CC[C@H](N2Cc3cc(N4CCN(CC5CCN(c6ccc([C@@H]7c8ccc(O)cc8CC[C@@H]7CC(F)(F)F)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7CC(F)(F)F)cc6)CC5)CC4)ccc3C2=O)C(=O)N1 ; O=C1CC[C@H](N2Cc3cc(N4CCN(CC5CCN(c6c(F)cc([C@@H]7c8ccc(O)cc8CC[C@@H]7C7CCCCC7)cc6F)CC5)CC4)ccc3C2=O)C(=O)N1] 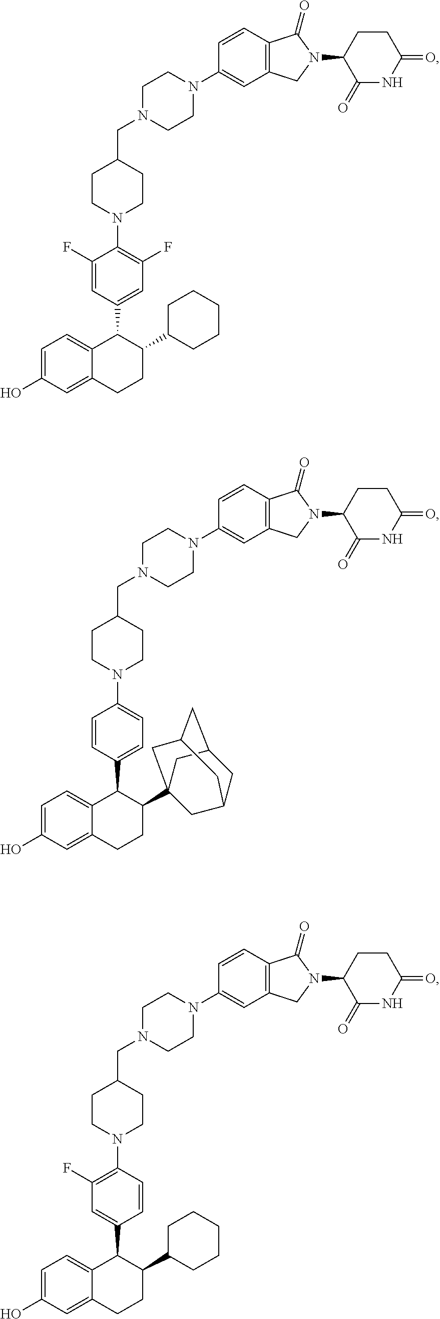 [SMILES: O=C1CC[C@H](N2Cc3cc(N4CCN(CC5CCN(c6c(F)cc([C@H]7c8ccc(O)cc8CC[C@H]7C7CCCCC7)cc6F)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C78CC9CC(CC(C9)C7)C8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7C7CCCCC7)cc6F)CC5)CC4)ccc3C2=O)C(=O)N1] 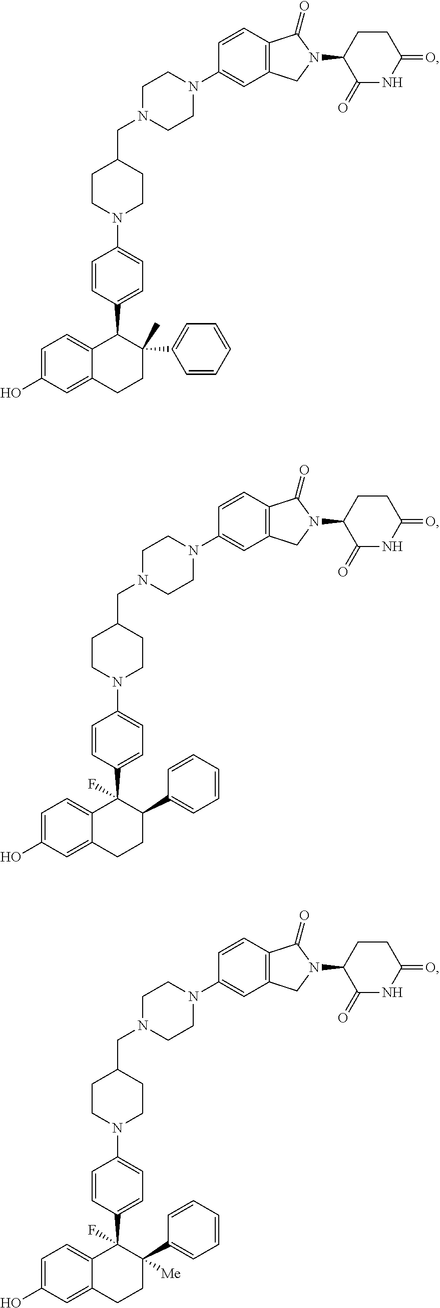 [SMILES: C[C@@]1(c2ccccc2)CCc2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@]7(F)c8ccc(O)cc8CC[C@@H]7c7ccccc7)cc6)CC5)CC4)ccc3C2=O)C(=O)N1 ; C[C@]1(c2ccccc2)CCc2cc(O)ccc2[C@@]1(F)c1ccc(N2CCC(CN3CCN(c4ccc5c(c4)CN([C@H]4CCC(=O)NC4=O)C5=O)CC3)CC2)cc1] 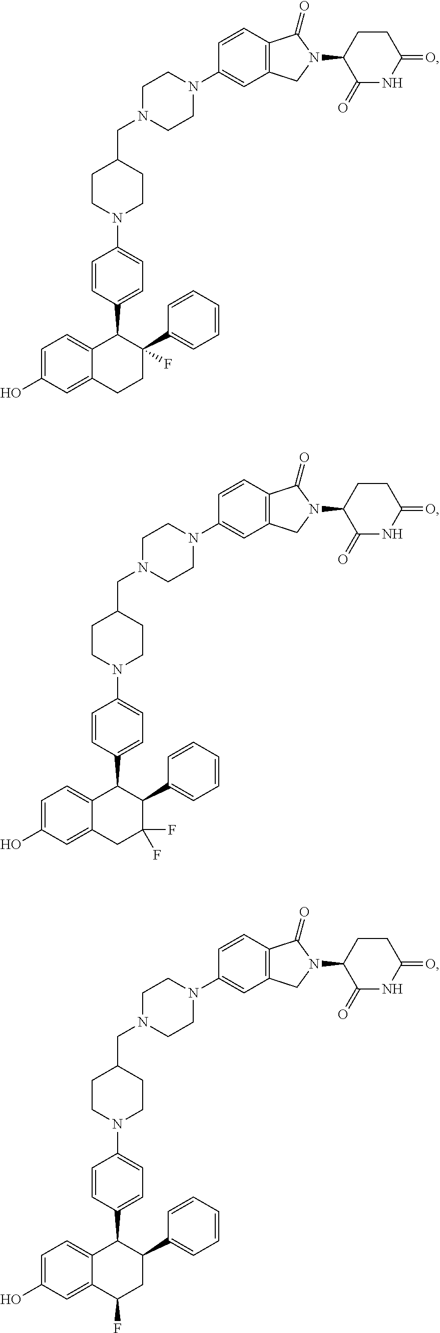 [SMILES: O=C1CC[C@H](N2Cc3cc(N4CCN(CC5CCN(c6ccc([C@@H]7c8ccc(O)cc8CC[C@]7(F)c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC(F)(F)[C@@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8[C@H](F)C[C@@H]7c7ccccc7)cc6)CC5)CC4)ccc3C2=O)C(=O)N1] 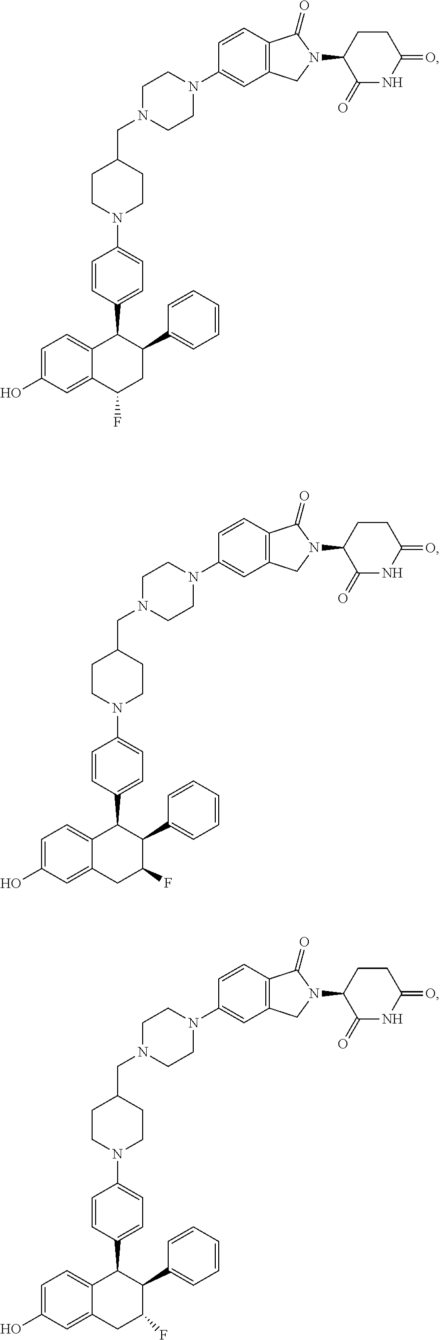 [SMILES: O=C1CC[C@H](N2Cc3cc(N4CCN(CC5CCN(c6ccc([C@@H]7c8ccc(O)cc8[C@@H](F)C[C@@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C[C@H](F)[C@@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C[C@@H](F)[C@@H]7c7ccccc7)cc6)CC5)CC4)ccc3C2=O)C(=O)N1] 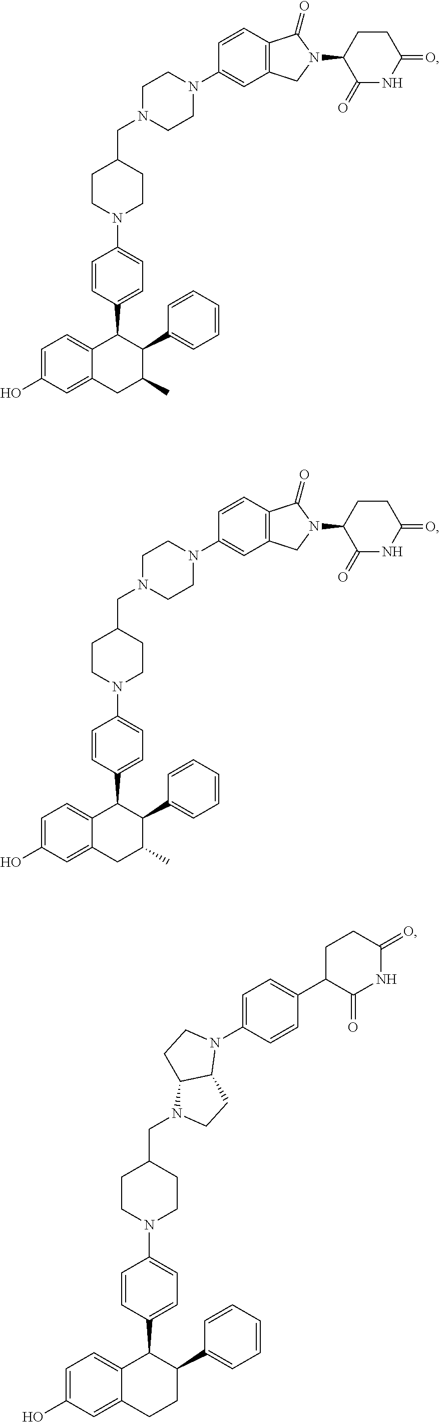 [SMILES: C[C@H]1Cc2cc(O)ccc2[C@@H](c2ccc(N3CCC(CN4CCN(c5ccc6c(c5)CN([C@H]5CCC(=O)NC5=O)C6=O)CC4)CC3)cc2)[C@H]1c1ccccc1 ; C[C@@H]1Cc2cc(O)ccc2[C@@H](c2ccc(N3CCC(CN4CCN(c5ccc6c(c5)CN([C@H]5CCC(=O)NC5=O)C6=O)CC4)CC3)cc2)[C@H]1c1ccccc1 ; O=C1CCC(c2ccc(N3CC[C@@H]4[C@H]3CCN4CC3CCN(c4ccc([C@@H]5c6ccc(O)cc6CC[C@@H]5c5ccccc5)cc4)CC3)cc2)C(=O)N1] 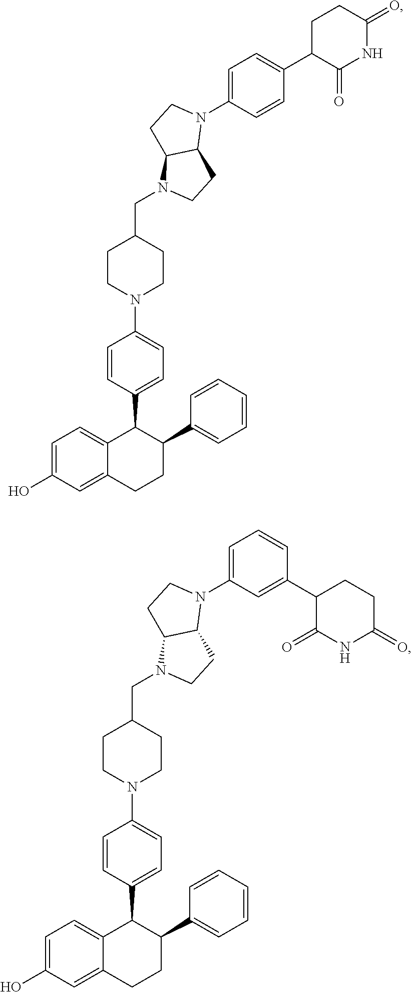 [SMILES: O=C1CCC(c2ccc(N3CC[C@H]4[C@@H]3CCN4CC3CCN(c4ccc([C@@H]5c6ccc(O)cc6CC[C@@H]5c5ccccc5)cc4)CC3)cc2)C(=O)N1 ; O=C1CCC(c2cccc(N3CC[C@@H]4[C@H]3CCN4CC3CCN(c4ccc([C@@H]5c6ccc(O)cc6CC[C@@H]5c5ccccc5)cc4)CC3)c2)C(=O)N1] 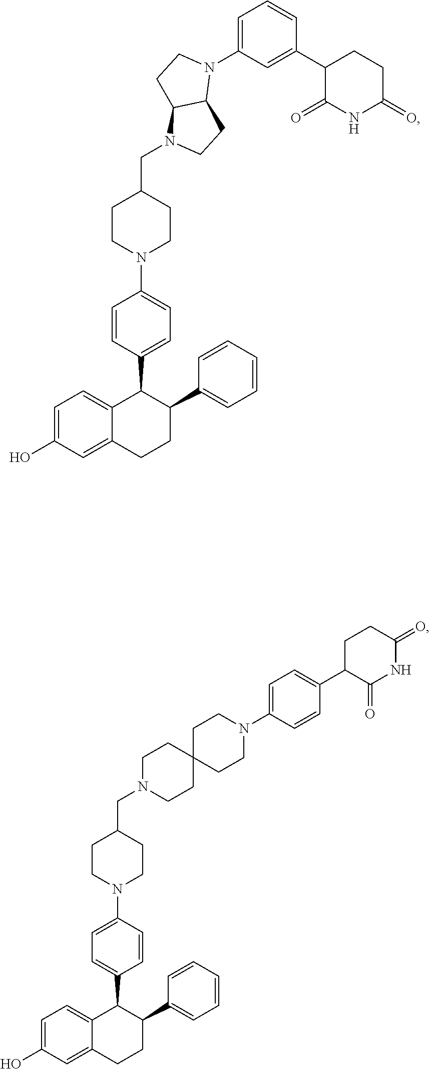 [SMILES: O=C1CCC(c2cccc(N3CC[C@H]4[C@@H]3CCN4CC3CCN(c4ccc([C@@H]5c6ccc(O)cc6CC[C@@H]5c5ccccc5)cc4)CC3)c2)C(=O)N1 ; O=C1CCC(c2ccc(N3CCC4(CCN(CC5CCN(c6ccc([C@@H]7c8ccc(O)cc8CC[C@@H]7c7ccccc7)cc6)CC5)CC4)CC3)cc2)C(=O)N1] 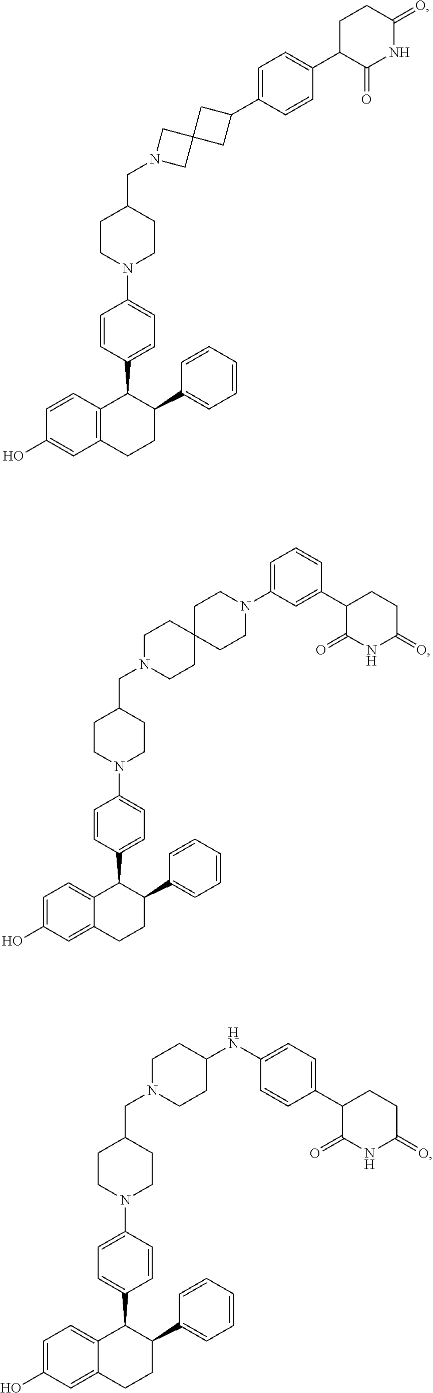 [SMILES: O=C1CCC(c2ccc(C3CC4(C3)CN(CC3CCN(c5ccc([C@@H]6c7ccc(O)cc7CC[C@@H]6c6ccccc6)cc5)CC3)C4)cc2)C(=O)N1 ; O=C1CCC(c2cccc(N3CCC4(CCN(CC5CCN(c6ccc([C@@H]7c8ccc(O)cc8CC[C@@H]7c7ccccc7)cc6)CC5)CC4)CC3)c2)C(=O)N1 ; O=C1CCC(c2ccc(NC3CCN(CC4CCN(c5ccc([C@@H]6c7ccc(O)cc7CC[C@@H]6c6ccccc6)cc5)CC4)CC3)cc2)C(=O)N1] 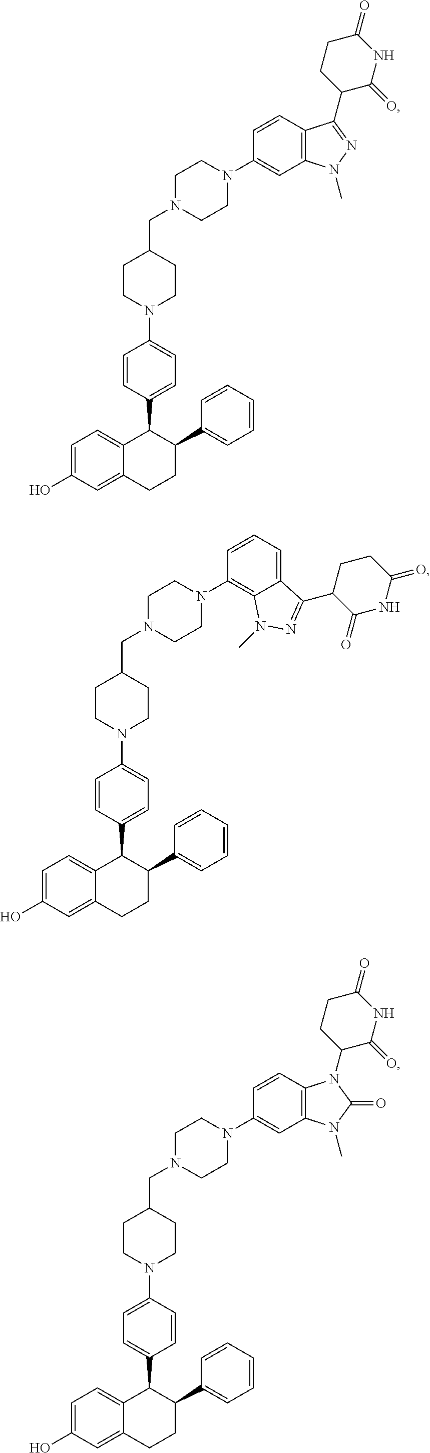 [SMILES: Cn1nc(C2CCC(=O)NC2=O)c2ccc(N3CCN(CC4CCN(c5ccc([C@@H]6c7ccc(O)cc7CC[C@@H]6c6ccccc6)cc5)CC4)CC3)cc21 ; Cn1nc(C2CCC(=O)NC2=O)c2cccc(N3CCN(CC4CCN(c5ccc([C@@H]6c7ccc(O)cc7CC[C@@H]6c6ccccc6)cc5)CC4)CC3)c21 ; Cn1c(=O)n(C2CCC(=O)NC2=O)c2ccc(N3CCN(CC4CCN(c5ccc([C@@H]6c7ccc(O)cc7CC[C@@H]6c6ccccc6)cc5)CC4)CC3)cc21] 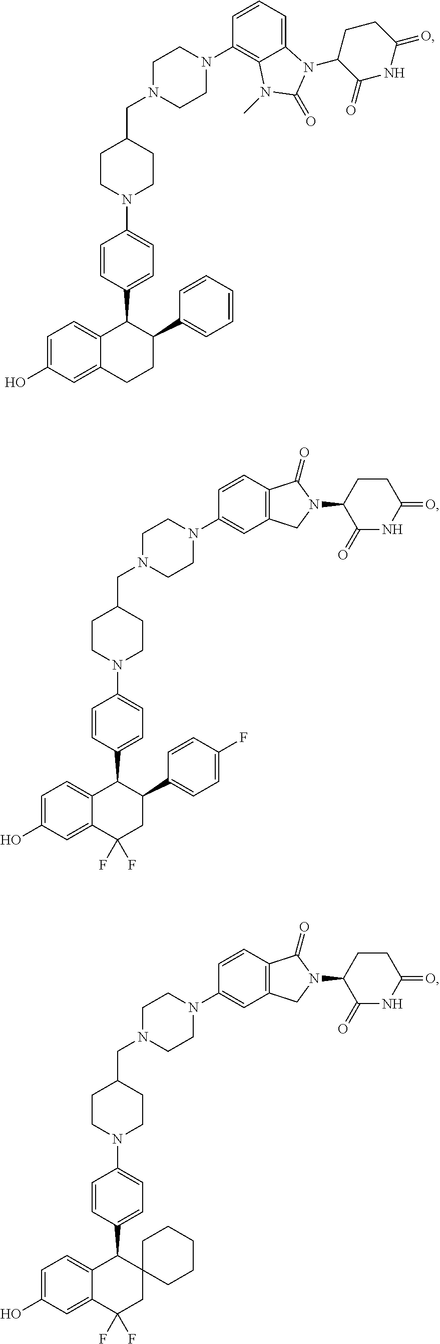 [SMILES: Cn1c(=O)n(C2CCC(=O)NC2=O)c2cccc(N3CCN(CC4CCN(c5ccc([C@@H]6c7ccc(O)cc7CC[C@@H]6c6ccccc6)cc5)CC4)CC3)c21 ; O=C1CC[C@H](N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)CC78CCCCC8)cc6)CC5)CC4)ccc3C2=O)C(=O)N1] 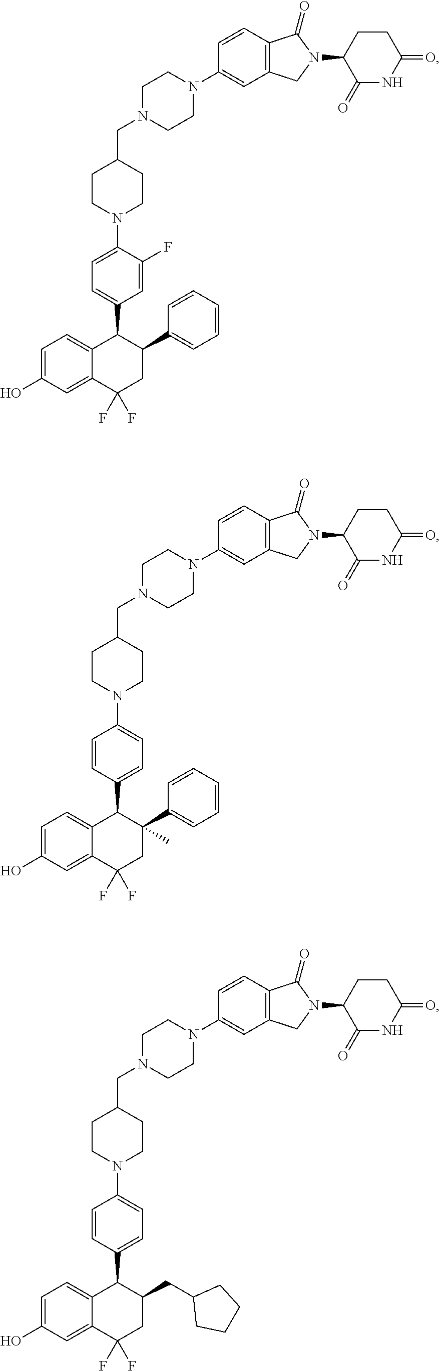 [SMILES: O=C1CC[C@H](N2Cc3cc(N4CCN(CC5CCN(c6ccc([C@@H]7c8ccc(O)cc8C(F)(F)C[C@@H]7c7ccccc7)cc6F)CC5)CC4)ccc3C2=O)C(=O)N1 ; C[C@]1(c2ccccc2)CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8C(F)(F)C[C@@H]7CC7CCCC7)cc6)CC5)CC4)ccc3C2=O)C(=O)N1] 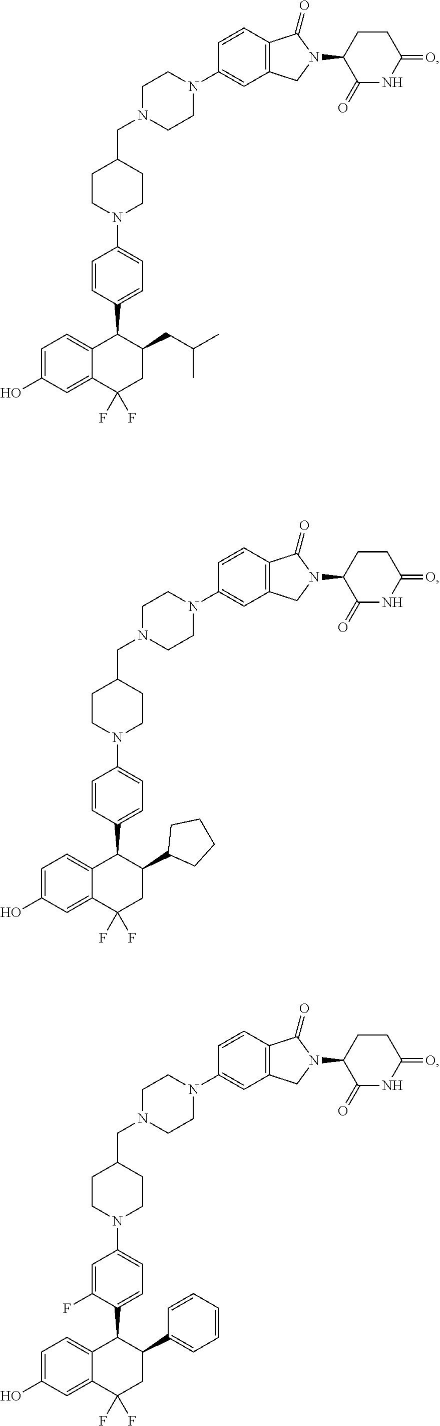 [SMILES: CC(C)C[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8C(F)(F)C[C@@H]7C7CCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccccc7)c(F)c6)CC5)CC4)ccc3C2=O)C(=O)N1] 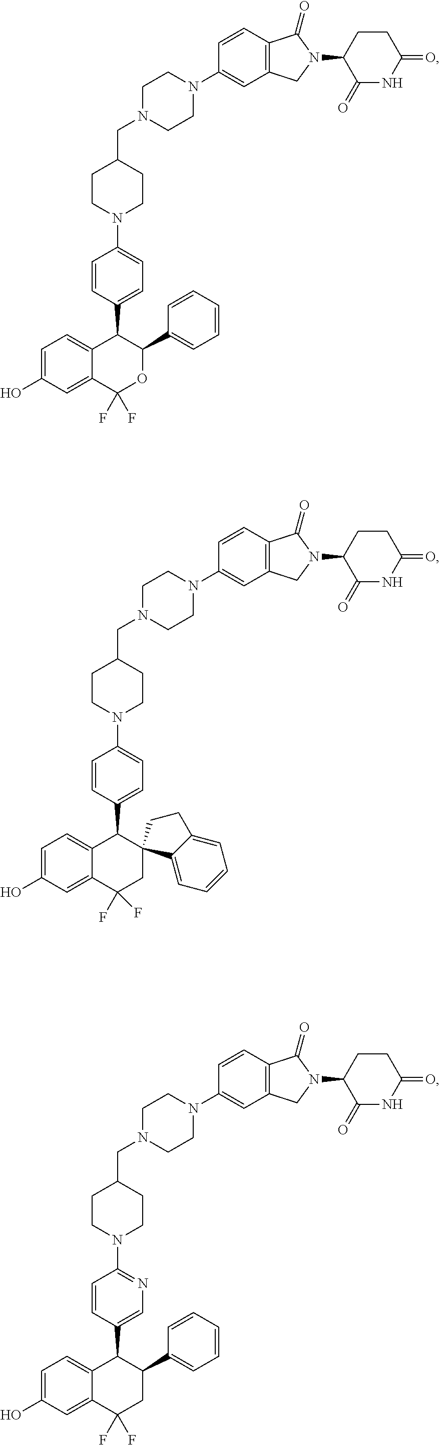 [SMILES: O=C1CC[C@H](N2Cc3cc(N4CCN(CC5CCN(c6ccc([C@@H]7c8ccc(O)cc8C(F)(F)O[C@@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@]78CCc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccccc7)cn6)CC5)CC4)ccc3C2=O)C(=O)N1] 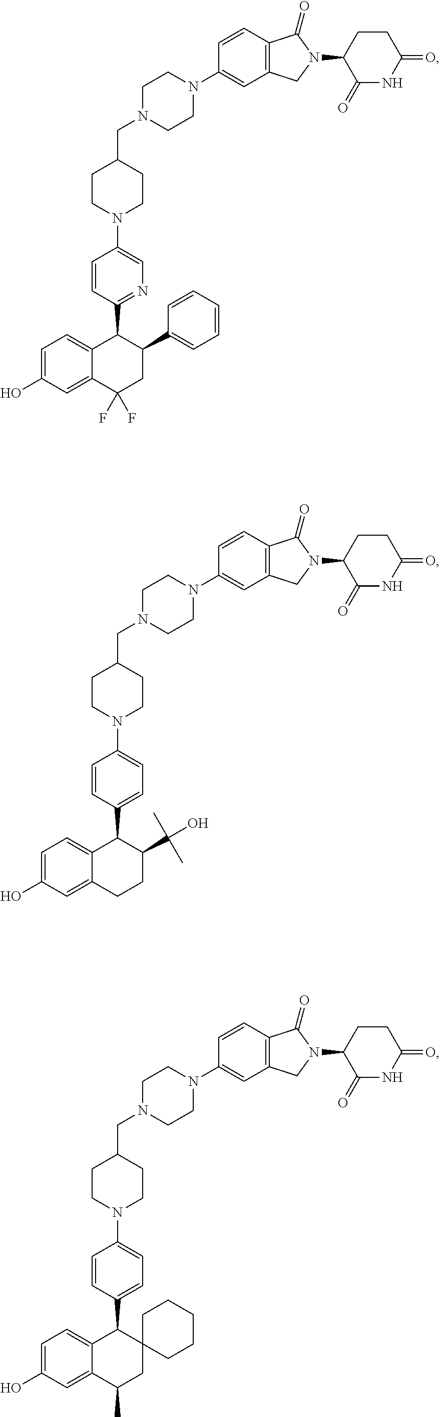 [SMILES: O=C1CC[C@H](N2Cc3cc(N4CCN(CC5CCN(c6ccc([C@@H]7c8ccc(O)cc8C(F)(F)C[C@@H]7c7ccccc7)nc6)CC5)CC4)ccc3C2=O)C(=O)N1 ; CC(C)(O)[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; C[C@@H]1CC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] 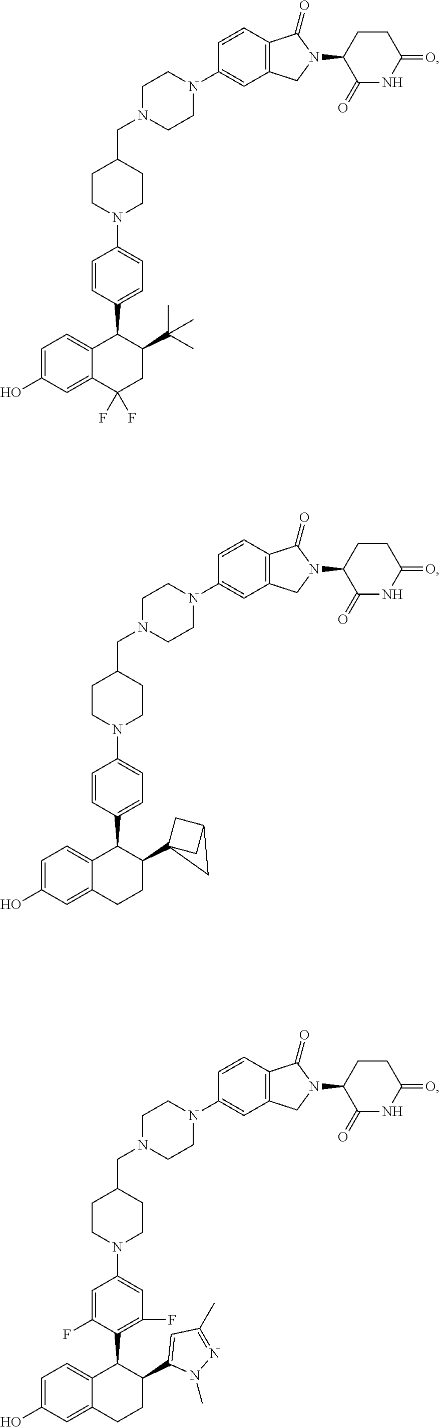 [SMILES: CC(C)(C)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8CC[C@@H]7C78CC(C7)C8)cc6)CC5)CC4)ccc3C2=O)C(=O)N1 ; Cc1cc([C@H]2CCc3cc(O)ccc3[C@H]2c2c(F)cc(N3CCC(CN4CCN(c5ccc6c(c5)CN([C@H]5CCC(=O)NC5=O)C6=O)CC4)CC3)cc2F)n(C)n1] 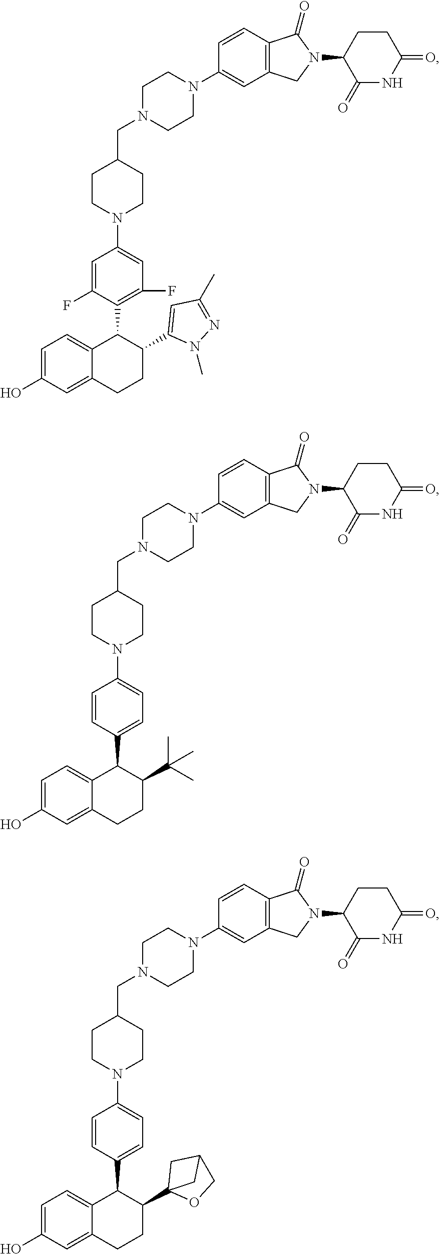 [SMILES: Cc1cc([C@@H]2CCc3cc(O)ccc3[C@@H]2c2c(F)cc(N3CCC(CN4CCN(c5ccc6c(c5)CN([C@H]5CCC(=O)NC5=O)C6=O)CC4)CC3)cc2F)n(C)n1 ; CC(C)(C)[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@@H]7c8ccc(O)cc8CC[C@@H]7C78CC(CO7)C8)cc6)CC5)CC4)ccc3C2=O)C(=O)N1] 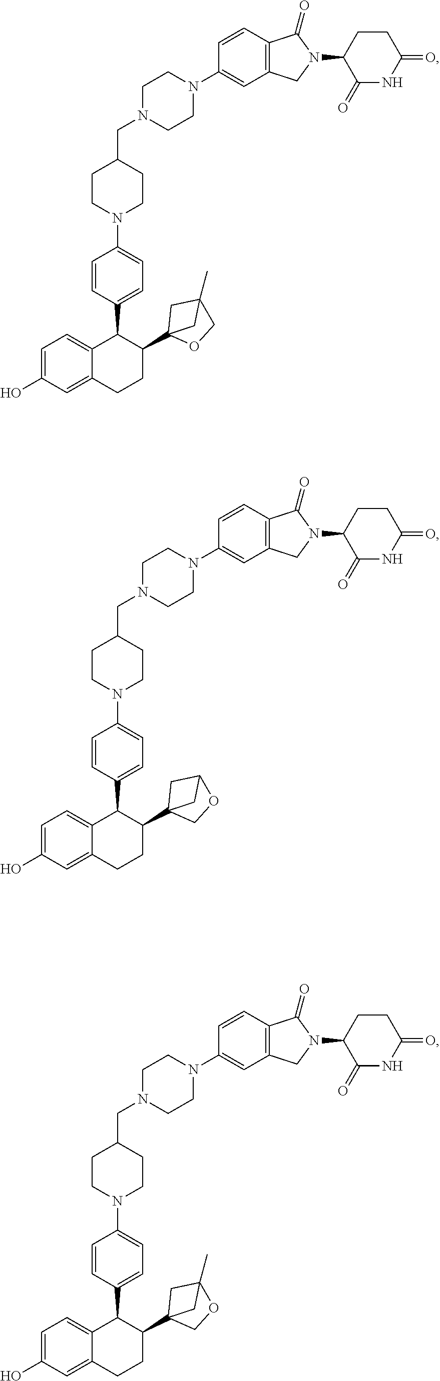 [SMILES: CC12COC([C@H]3CCc4cc(O)ccc4[C@H]3c3ccc(N4CCC(CN5CCN(c6ccc7c(c6)CN([C@H]6CCC(=O)NC6=O)C7=O)CC5)CC4)cc3)(C1)C2 ; O=C1CC[C@H](N2Cc3cc(N4CCN(CC5CCN(c6ccc([C@@H]7c8ccc(O)cc8CC[C@@H]7C78COC(C7)C8)cc6)CC5)CC4)ccc3C2=O)C(=O)N1 ; CC12CC([C@H]3CCc4cc(O)ccc4[C@H]3c3ccc(N4CCC(CN5CCN(c6ccc7c(c6)CN([C@H]6CCC(=O)NC6=O)C7=O)CC5)CC4)cc3)(CO1)C2] 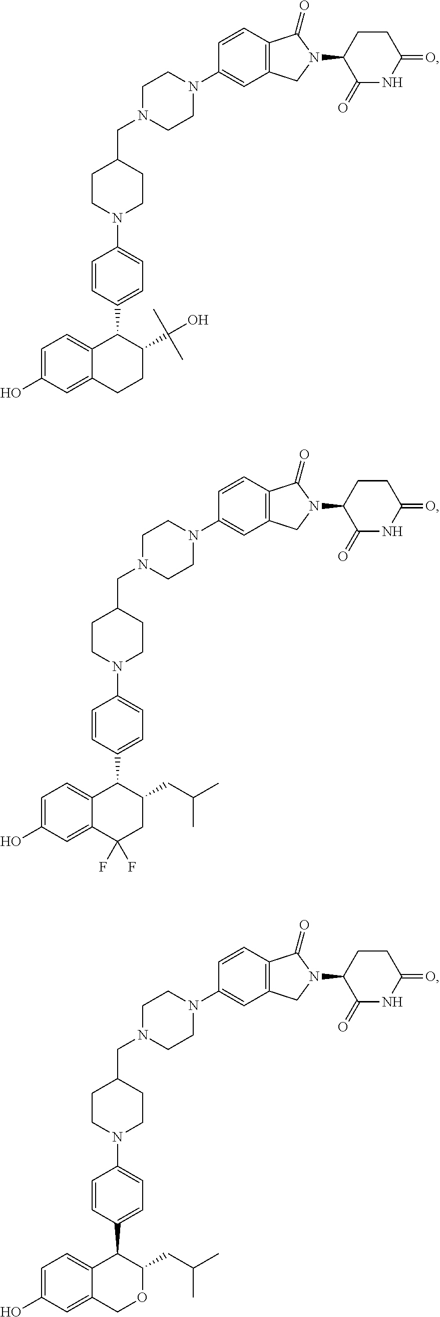 [SMILES: CC(C)(O)[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; CC(C)C[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1 ; CC(C)C[C@@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] 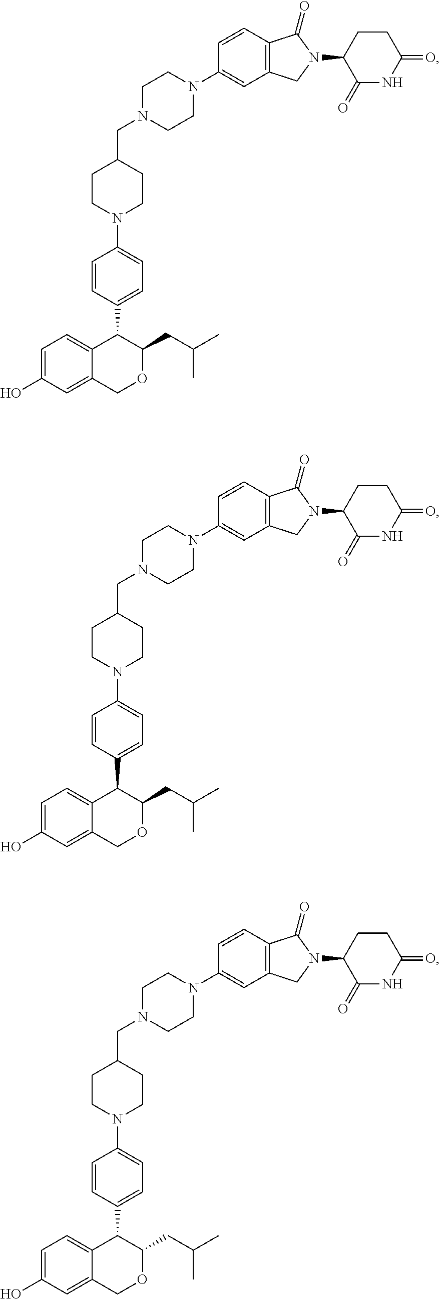 [SMILES: CC(C)C[C@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; CC(C)C[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1 ; CC(C)C[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] 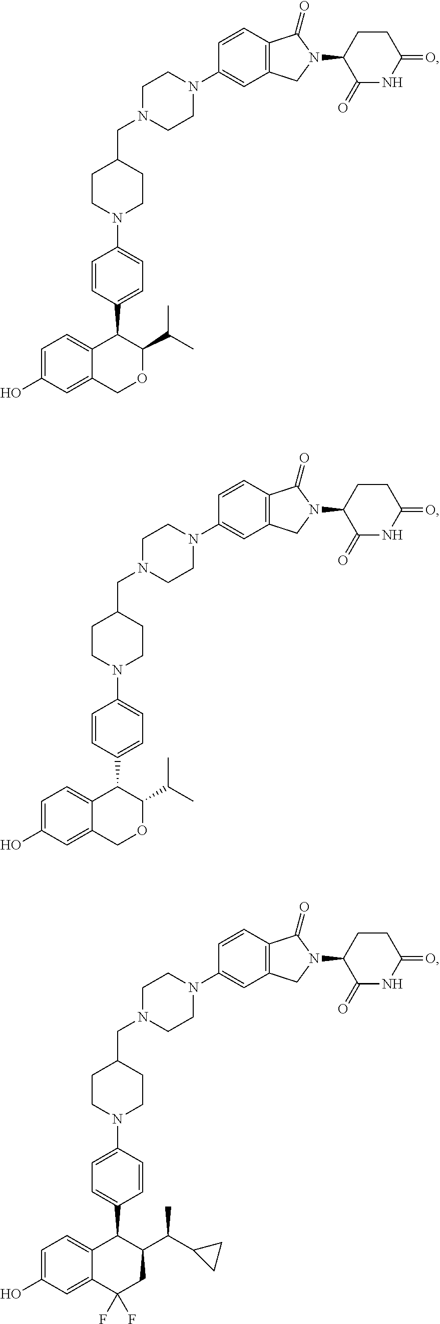 [SMILES: CC(C)[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1 ; CC(C)[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; C[C@@H](C1CC1)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] 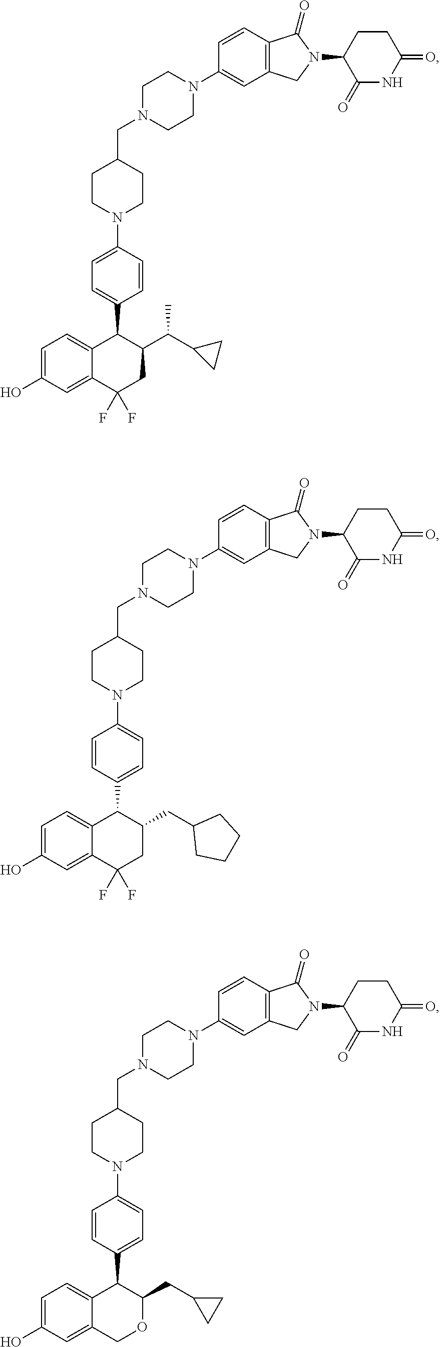 [SMILES: C[C@H](C1CC1)[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@H]7c8ccc(O)cc8C(F)(F)C[C@H]7CC7CCCC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7CC7CC7)cc6)CC5)CC4)ccc3C2=O)C(=O)N1] 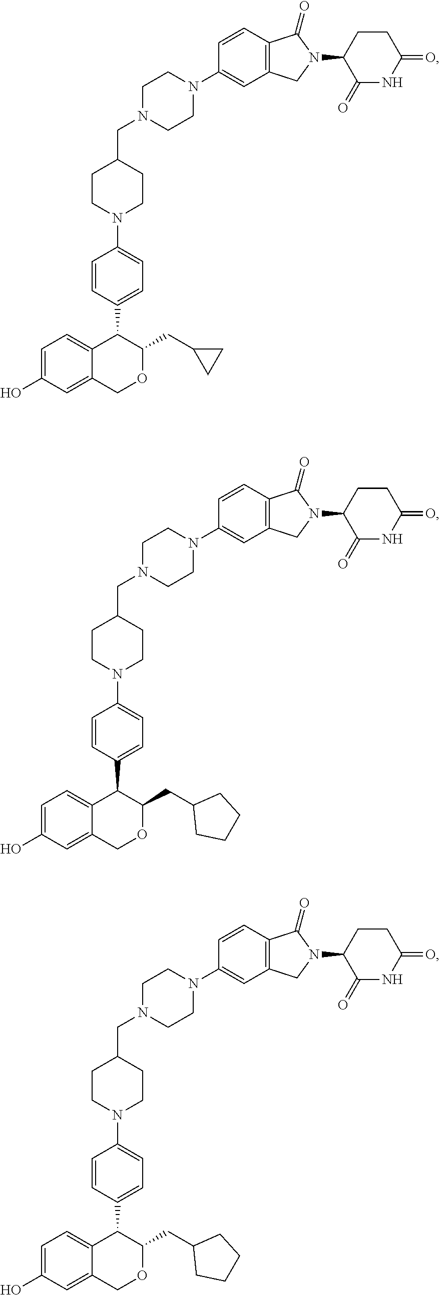 [SMILES: O=C1CC[C@H](N2Cc3cc(N4CCN(CC5CCN(c6ccc([C@H]7c8ccc(O)cc8CO[C@H]7CC7CC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7CC7CCCC7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7CC7CCCC7)cc6)CC5)CC4)ccc3C2=O)C(=O)N1] 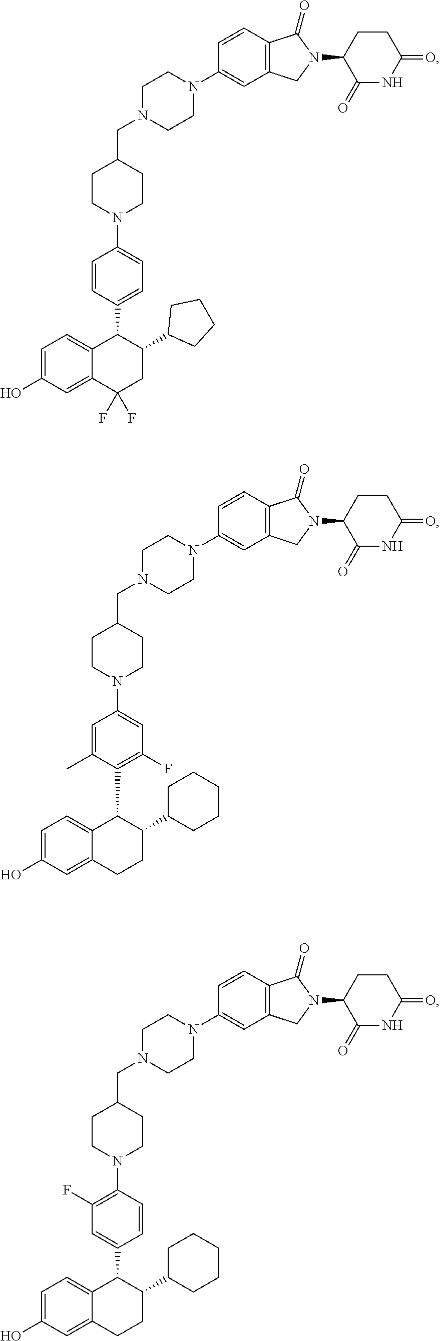 [SMILES: O=C1CC[C@H](N2Cc3cc(N4CCN(CC5CCN(c6ccc([C@H]7c8ccc(O)cc8C(F)(F)C[C@H]7C7CCCC7)cc6)CC5)CC4)ccc3C2=O)C(=O)N1 ; Cc1cc(N2CCC(CN3CCN(c4ccc5c(c4)CN([C@H]4CCC(=O)NC4=O)C5=O)CC3)CC2)cc(F)c1[C@H]1c2ccc(O)cc2CC[C@H]1C1CCCCC1 ; O=C1CC[C@H](N2Cc3cc(N4CCN(CC5CCN(c6ccc([C@H]7c8ccc(O)cc8CC[C@H]7C7CCCCC7)cc6F)CC5)CC4)ccc3C2=O)C(=O)N1] 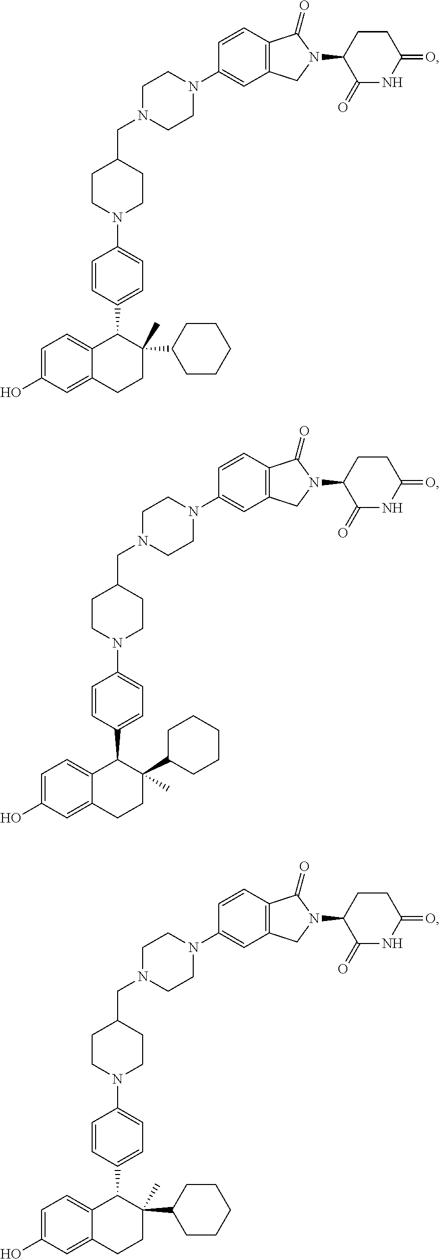 [SMILES: C[C@@]1(C2CCCCC2)CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; C[C@]1(C2CCCCC2)CCc2cc(O)ccc2[C@H]1c1ccc(N2CCC(CN3CCN(c4ccc5c(c4)CN([C@H]4CCC(=O)NC4=O)C5=O)CC3)CC2)cc1 ; C[C@]1(C2CCCCC2)CCc2cc(O)ccc2[C@@H]1c1ccc(N2CCC(CN3CCN(c4ccc5c(c4)CN([C@H]4CCC(=O)NC4=O)C5=O)CC3)CC2)cc1] 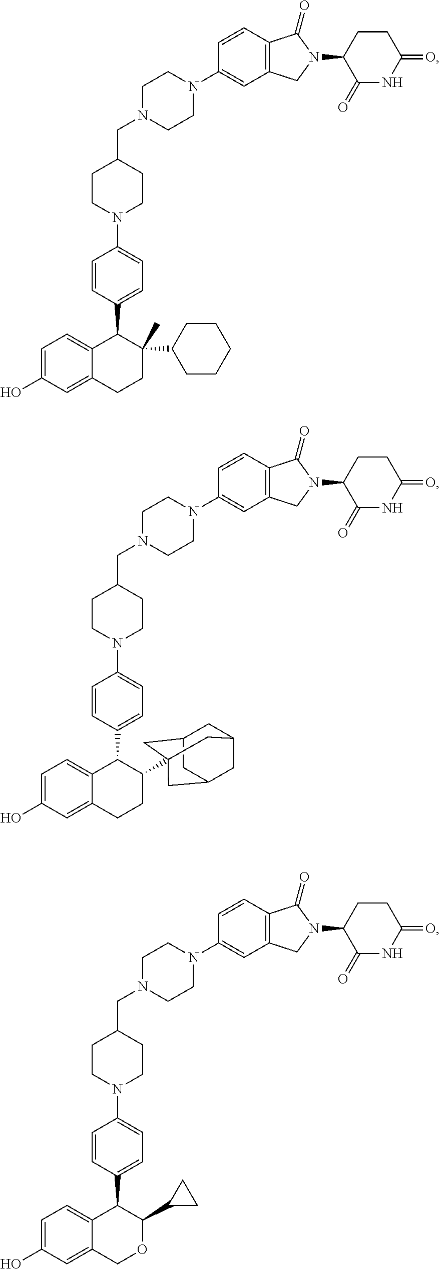 [SMILES: C[C@@]1(C2CCCCC2)CCc2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@H]7c8ccc(O)cc8CC[C@H]7C78CC9CC(CC(C9)C7)C8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7C7CC7)cc6)CC5)CC4)ccc3C2=O)C(=O)N1] 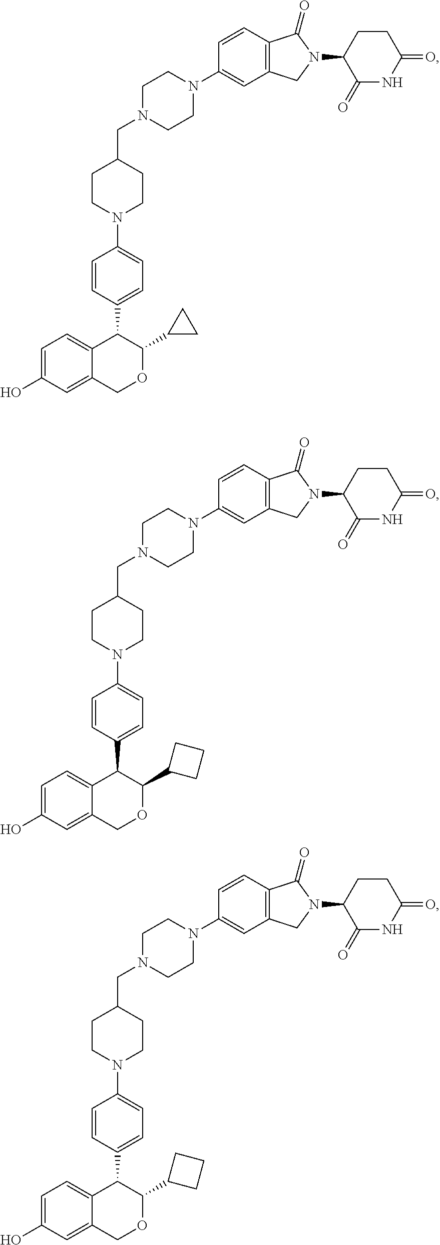 [SMILES: O=C1CC[C@H](N2Cc3cc(N4CCN(CC5CCN(c6ccc([C@H]7c8ccc(O)cc8CO[C@H]7C7CC7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7C7CCC7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7C7CCC7)cc6)CC5)CC4)ccc3C2=O)C(=O)N1] 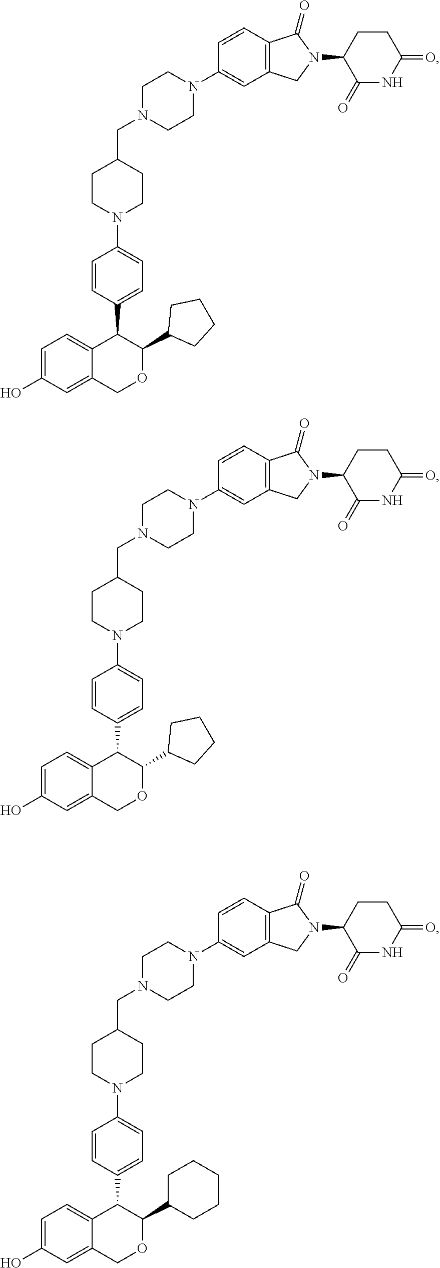 [SMILES: O=C1CC[C@H](N2Cc3cc(N4CCN(CC5CCN(c6ccc([C@@H]7c8ccc(O)cc8CO[C@@H]7C7CCCC7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7C7CCCC7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@@H]7C7CCCCC7)cc6)CC5)CC4)ccc3C2=O)C(=O)N1] 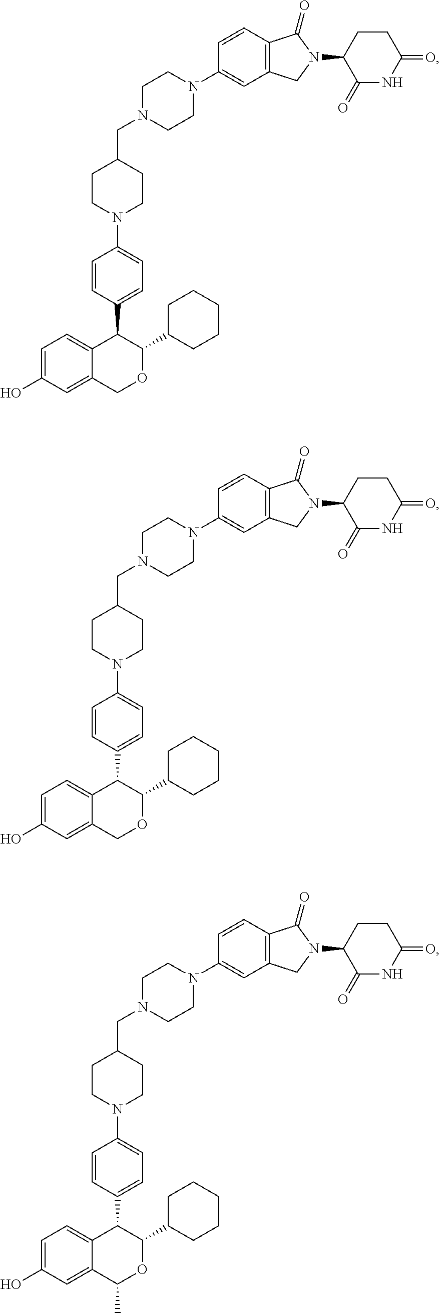 [SMILES: O=C1CC[C@H](N2Cc3cc(N4CCN(CC5CCN(c6ccc([C@@H]7c8ccc(O)cc8CO[C@H]7C7CCCCC7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7C7CCCCC7)cc6)CC5)CC4)ccc3C2=O)C(=O)N1 ; C[C@H]1O[C@@H](C2CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21] 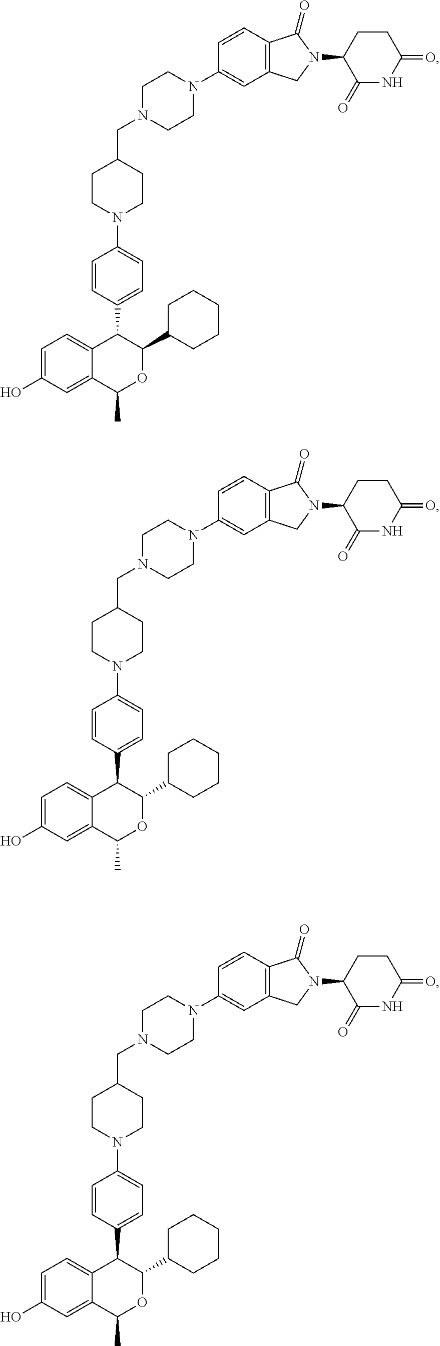 [SMILES: C[C@@H]1O[C@H](C2CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21 ; C[C@H]1O[C@@H](C2CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21 ; C[C@@H]1O[C@@H](C2CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] 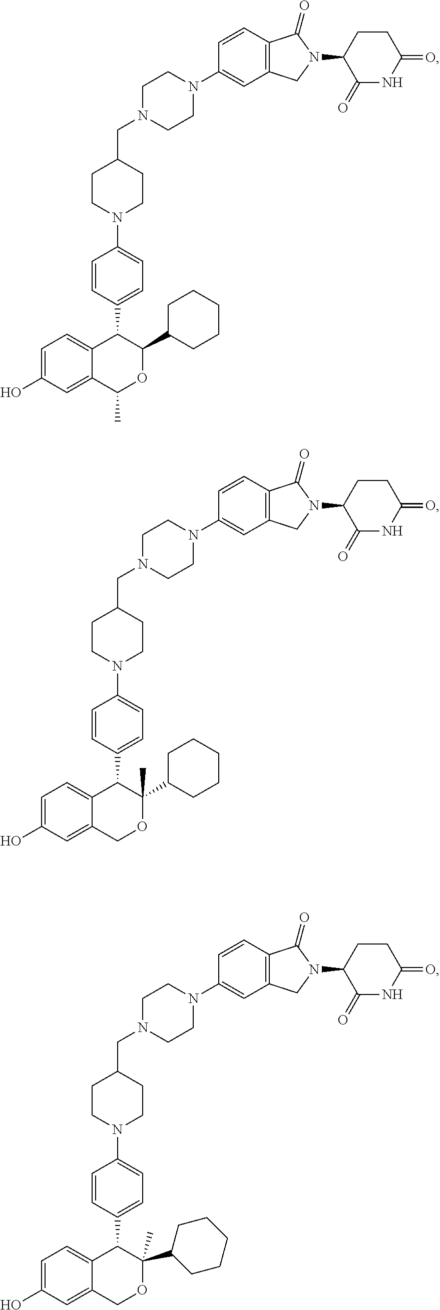 [SMILES: C[C@H]1O[C@H](C2CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21 ; C[C@@]1(C2CCCCC2)OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; C[C@]1(C2CCCCC2)OCc2cc(O)ccc2[C@@H]1c1ccc(N2CCC(CN3CCN(c4ccc5c(c4)CN([C@H]4CCC(=O)NC4=O)C5=O)CC3)CC2)cc1] 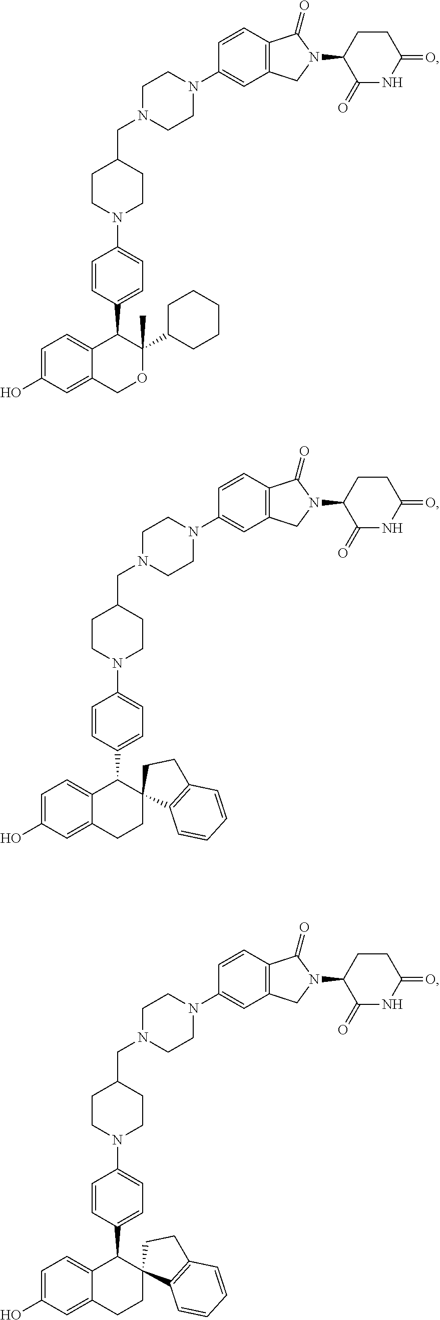 [SMILES: C[C@@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@H]7c8ccc(O)cc8CC[C@]78CCc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@]78CCc7ccccc78)cc6)CC5)CC4)ccc3C2=O)C(=O)N1] 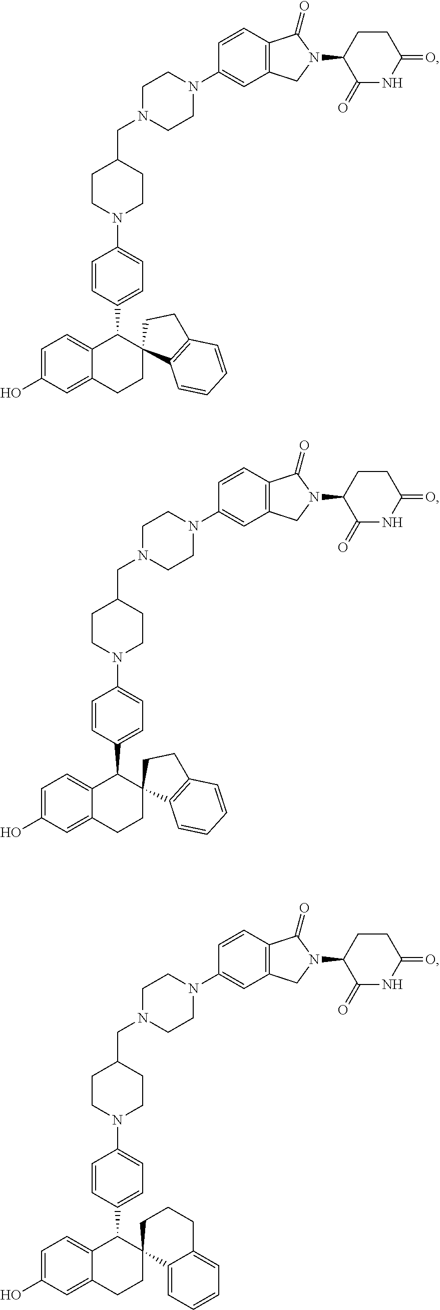 [SMILES: O=C1CC[C@H](N2Cc3cc(N4CCN(CC5CCN(c6ccc([C@H]7c8ccc(O)cc8CC[C@@]78CCc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@]78CCc7ccccc78)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@]78CCCc7ccccc78)cc6)CC5)CC4)ccc3C2=O)C(=O)N1] 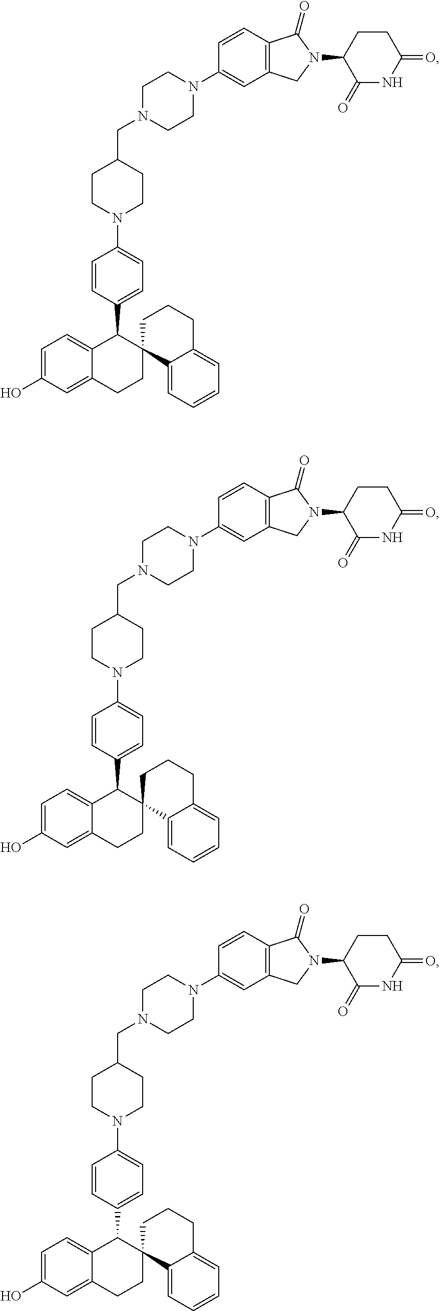 [SMILES: O=C1CC[C@H](N2Cc3cc(N4CCN(CC5CCN(c6ccc([C@@H]7c8ccc(O)cc8CC[C@@]78CCCc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@]78CCCc7ccccc78)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@@]78CCCc7ccccc78)cc6)CC5)CC4)ccc3C2=O)C(=O)N1] 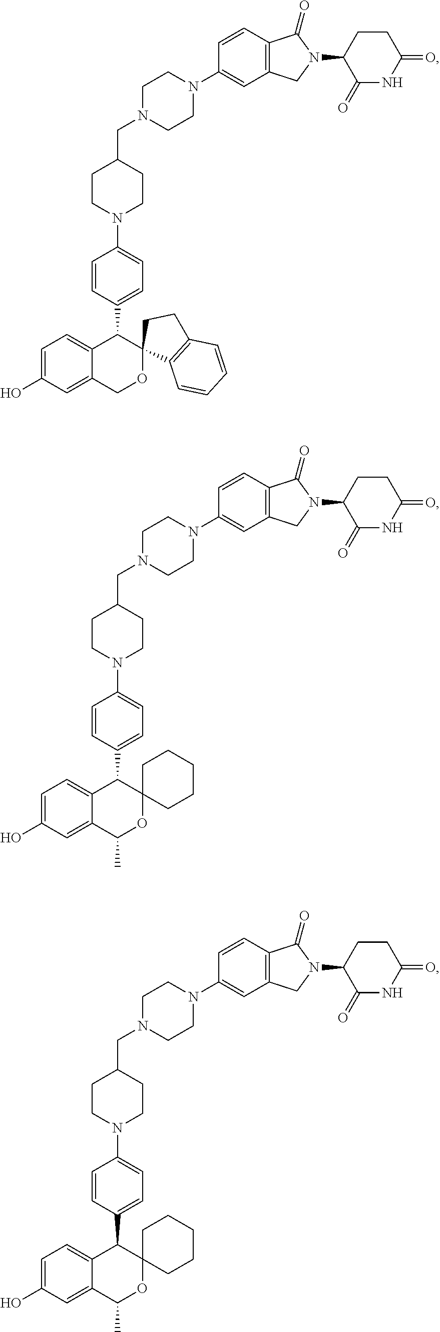 [SMILES: O=C1CC[C@H](N2Cc3cc(N4CCN(CC5CCN(c6ccc([C@H]7c8ccc(O)cc8CO[C@]78CCc7ccccc78)cc6)CC5)CC4)ccc3C2=O)C(=O)N1 ; C[C@H]1OC2(CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21 ; C[C@H]1OC2(CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21] 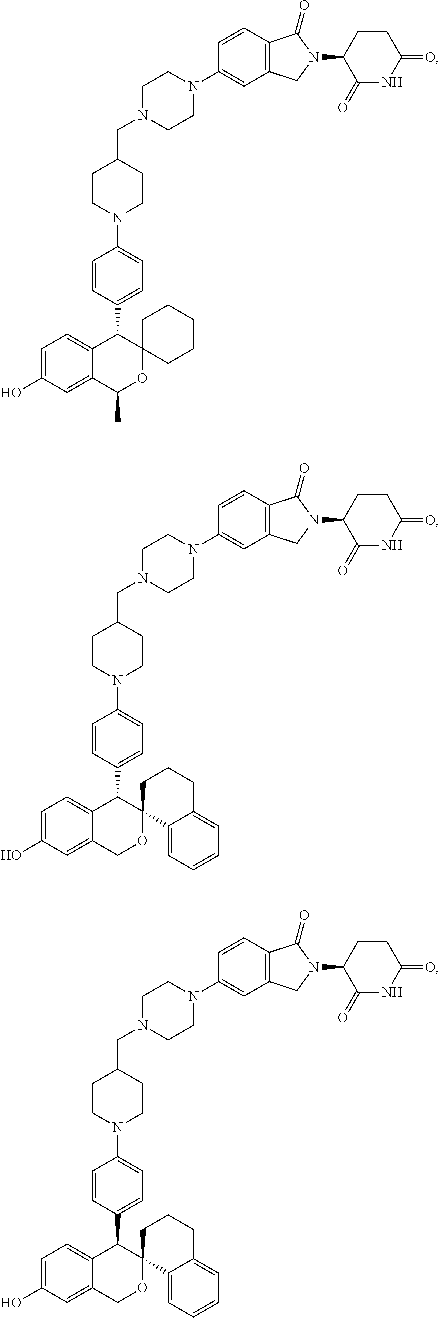 [SMILES: C[C@@H]1OC2(CCCCC2)[C@@H](c2ccc(N3CCC(CN4CCN(c5ccc6c(c5)CN([C@H]5CCC(=O)NC5=O)C6=O)CC4)CC3)cc2)c2ccc(O)cc21 ; O=C1CC[C@H](N2Cc3cc(N4CCN(CC5CCN(c6ccc([C@H]7c8ccc(O)cc8CO[C@]78CCCc7ccccc78)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@]78CCCc7ccccc78)cc6)CC5)CC4)ccc3C2=O)C(=O)N1] 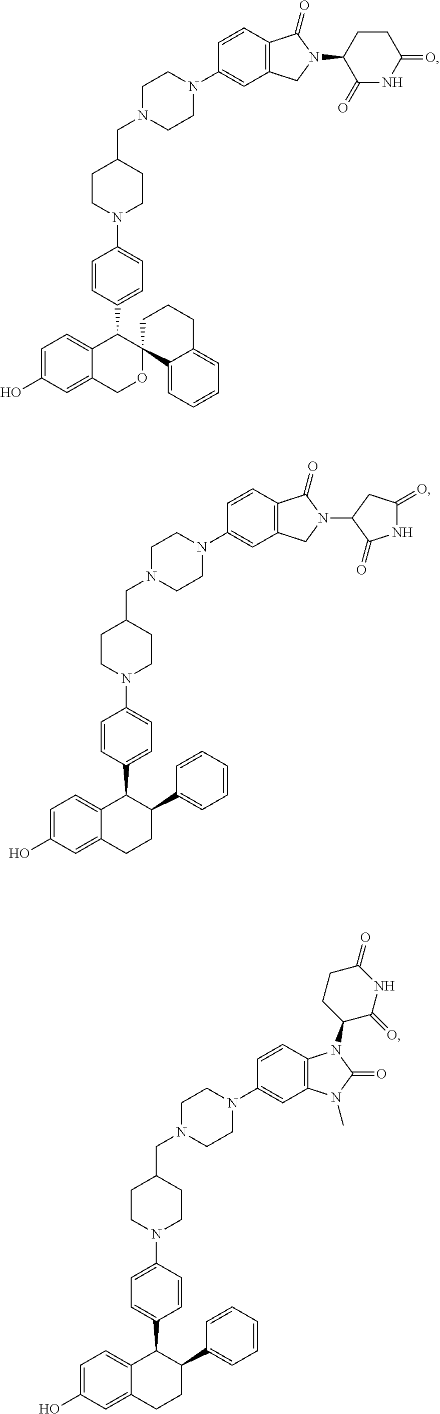 [SMILES: O=C1CC[C@H](N2Cc3cc(N4CCN(CC5CCN(c6ccc([C@H]7c8ccc(O)cc8CO[C@@]78CCCc7ccccc78)cc6)CC5)CC4)ccc3C2=O)C(=O)N1 ; O=C1CC(N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1 ; Cn1c(=O)n([C@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21] 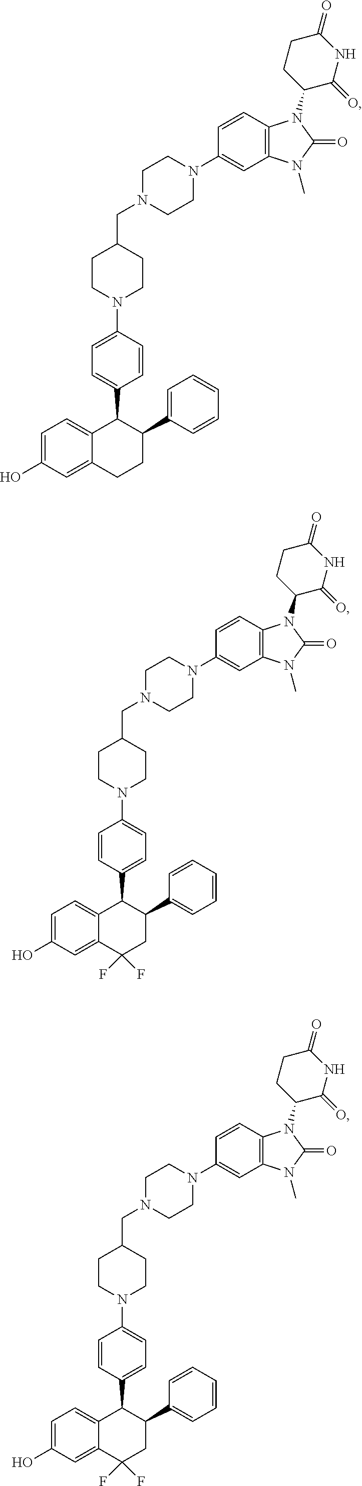 [SMILES: Cn1c(=O)n([C@@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21 ; Cn1c(=O)n([C@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21 ; Cn1c(=O)n([C@@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] 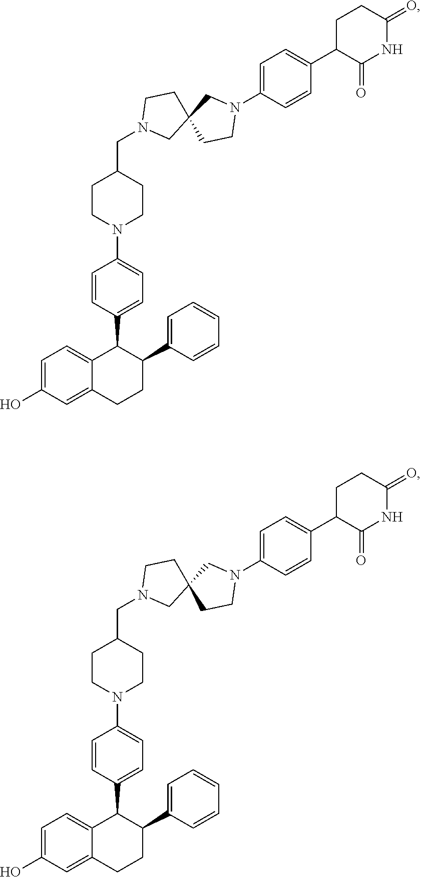 [SMILES: O=C1CCC(c2ccc(N3CC[C@]4(CCN(CC5CCN(c6ccc([C@@H]7c8ccc(O)cc8CC[C@@H]7c7ccccc7)cc6)CC5)C4)C3)cc2)C(=O)N1 ; O=C1CCC(c2ccc(N3CC[C@@]4(CCN(CC5CCN(c6ccc([C@@H]7c8ccc(O)cc8CC[C@@H]7c7ccccc7)cc6)CC5)C4)C3)cc2)C(=O)N1] 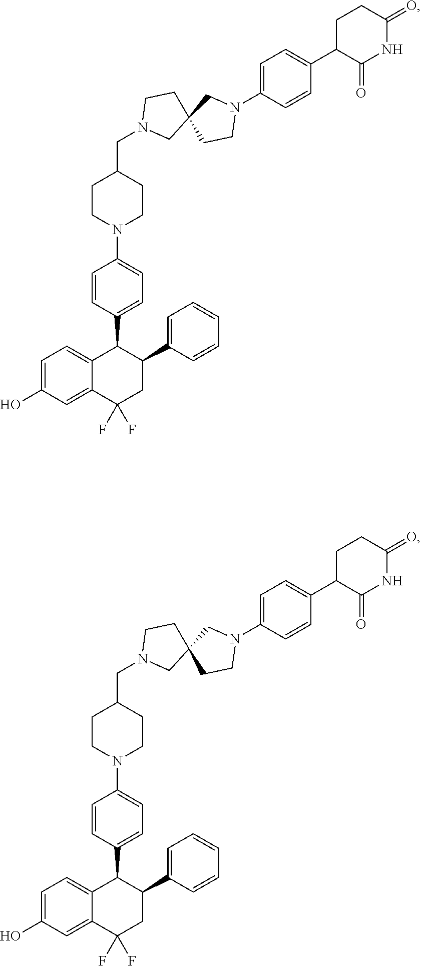 [SMILES: O=C1CCC(c2ccc(N3CC[C@]4(CCN(CC5CCN(c6ccc([C@@H]7c8ccc(O)cc8C(F)(F)C[C@@H]7c7ccccc7)cc6)CC5)C4)C3)cc2)C(=O)N1 ; O=C1CCC(c2ccc(N3CC[C@@]4(CCN(CC5CCN(c6ccc([C@@H]7c8ccc(O)cc8C(F)(F)C[C@@H]7c7ccccc7)cc6)CC5)C4)C3)cc2)C(=O)N1] 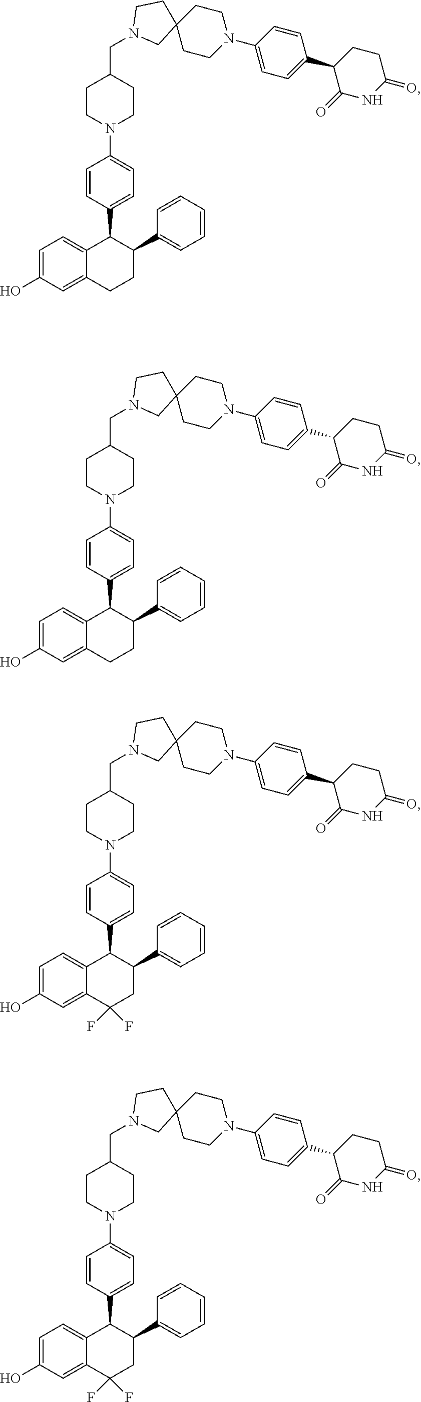 [SMILES: O=C1CC[C@H](c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1 ; O=C1CC[C@@H](c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1 ; O=C1CC[C@H](c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1 ; O=C1CC[C@@H](c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1] 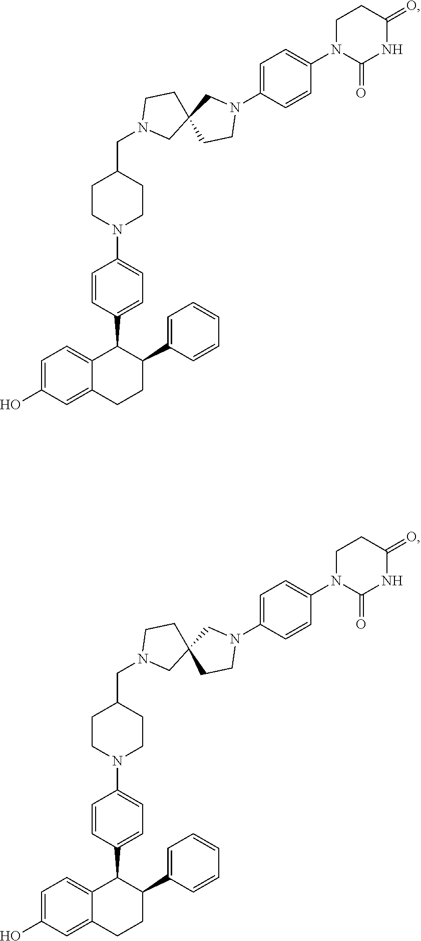 [SMILES: O=C1CCN(c2ccc(N3CC[C@]4(CCN(CC5CCN(c6ccc([C@@H]7c8ccc(O)cc8CC[C@@H]7c7ccccc7)cc6)CC5)C4)C3)cc2)C(=O)N1 ; O=C1CCN(c2ccc(N3CC[C@@]4(CCN(CC5CCN(c6ccc([C@@H]7c8ccc(O)cc8CC[C@@H]7c7ccccc7)cc6)CC5)C4)C3)cc2)C(=O)N1] 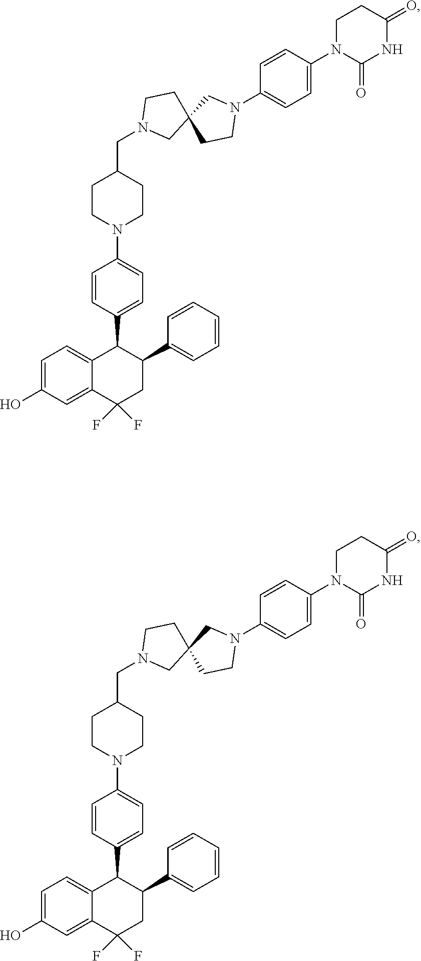 [SMILES: O=C1CCN(c2ccc(N3CC[C@@]4(CCN(CC5CCN(c6ccc([C@@H]7c8ccc(O)cc8C(F)(F)C[C@@H]7c7ccccc7)cc6)CC5)C4)C3)cc2)C(=O)N1 ; O=C1CCN(c2ccc(N3CC[C@]4(CCN(CC5CCN(c6ccc([C@@H]7c8ccc(O)cc8C(F)(F)C[C@@H]7c7ccccc7)cc6)CC5)C4)C3)cc2)C(=O)N1] 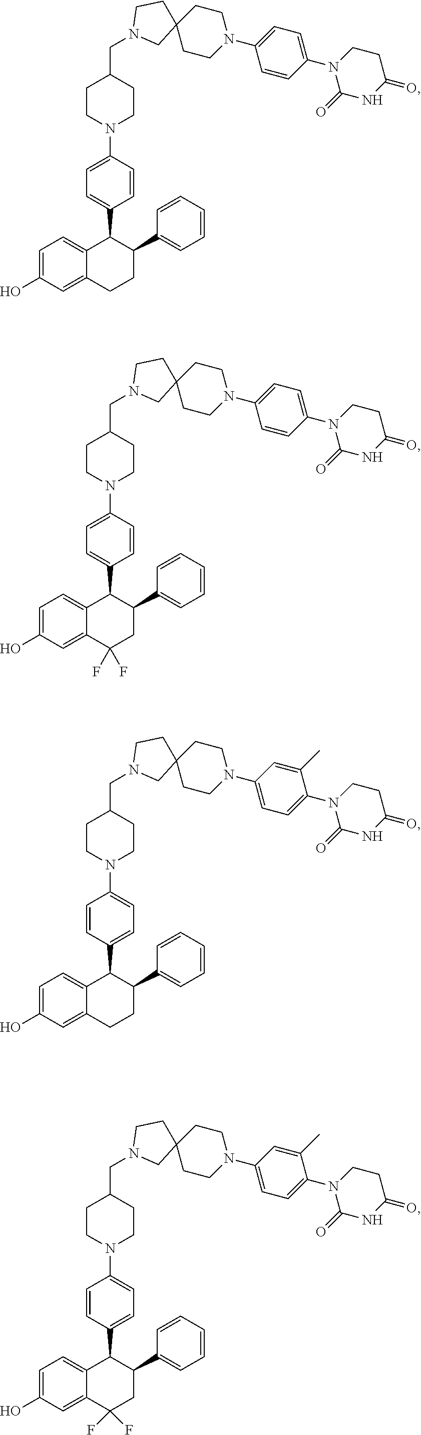 [SMILES: O=C1CCN(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1 ; O=C1CCN(c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1 ; Cc1cc(N2CCC3(CCN(CC4CCN(c5ccc([C@@H]6c7ccc(O)cc7CC[C@@H]6c6ccccc6)cc5)CC4)C3)CC2)ccc1N1CCC(=O)NC1=O ; Cc1cc(N2CCC3(CCN(CC4CCN(c5ccc([C@@H]6c7ccc(O)cc7C(F)(F)C[C@@H]6c6ccccc6)cc5)CC4)C3)CC2)ccc1N1CCC(=O)NC1=O] 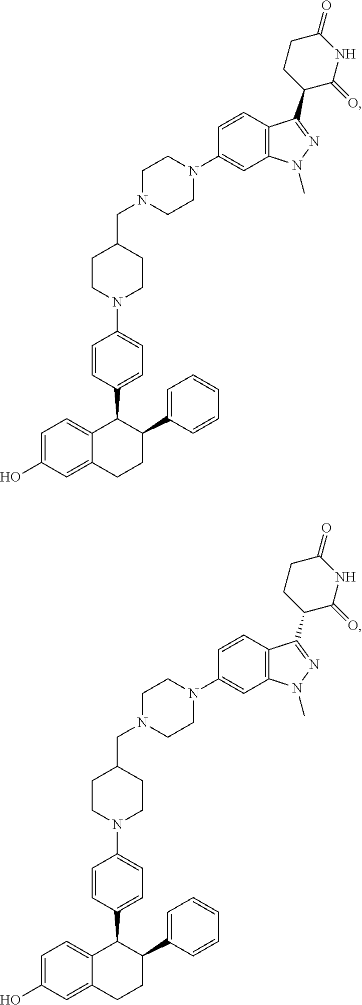 [SMILES: Cn1nc([C@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21 ; Cn1nc([C@@H]2CCC(=O)NC2=O)c2ccc(N3CCN(CC4CCN(c5ccc([C@@H]6c7ccc(O)cc7CC[C@@H]6c6ccccc6)cc5)CC4)CC3)cc21] 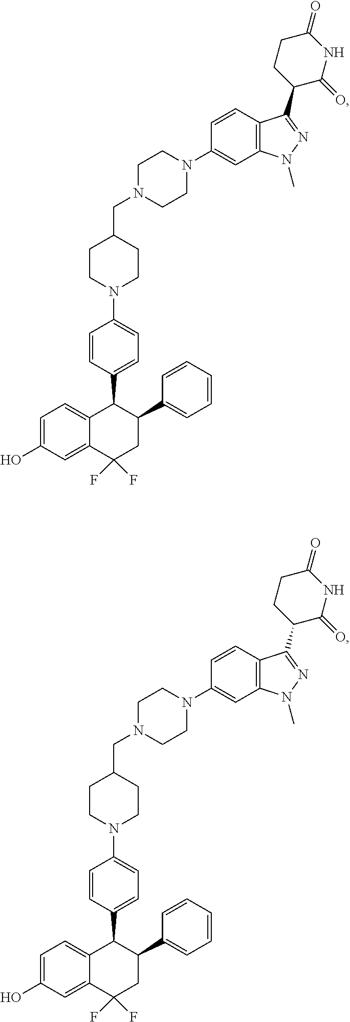 [SMILES: Cn1nc([C@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21 ; Cn1nc([C@@H]2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] 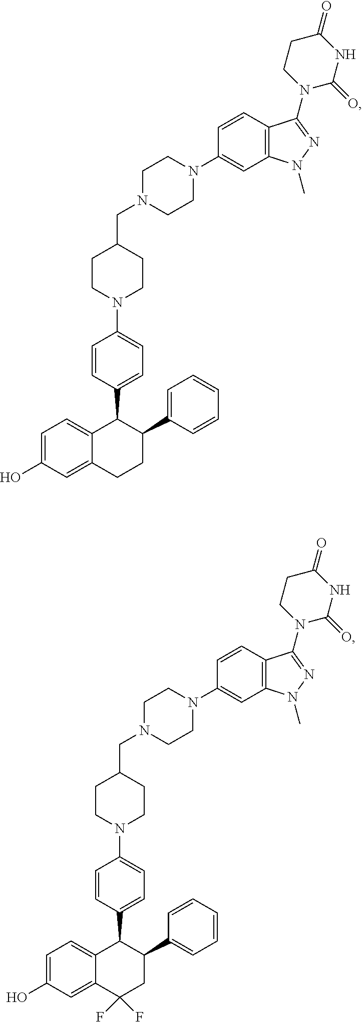 [SMILES: Cn1nc(N2CCC(=O)NC2=O)c2ccc(N3CCN(CC4CCN(c5ccc([C@@H]6c7ccc(O)cc7CC[C@@H]6c6ccccc6)cc5)CC4)CC3)cc21 ; Cn1nc(N2CCC(=O)NC2=O)c2ccc(N3CCN(CC4CCN(c5ccc([C@@H]6c7ccc(O)cc7C(F)(F)C[C@@H]6c6ccccc6)cc5)CC4)CC3)cc21] 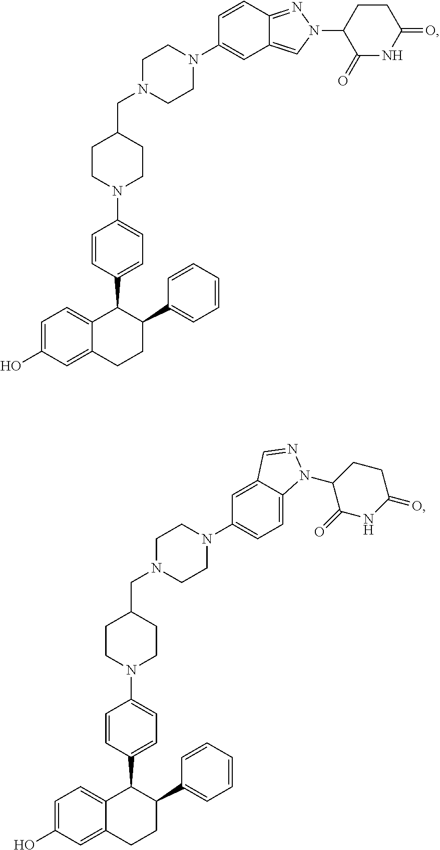 [SMILES: O=C1CCC(n2cc3cc(N4CCN(CC5CCN(c6ccc([C@@H]7c8ccc(O)cc8CC[C@@H]7c7ccccc7)cc6)CC5)CC4)ccc3n2)C(=O)N1 ; O=C1CCC(n2ncc3cc(N4CCN(CC5CCN(c6ccc([C@@H]7c8ccc(O)cc8CC[C@@H]7c7ccccc7)cc6)CC5)CC4)ccc32)C(=O)N1] 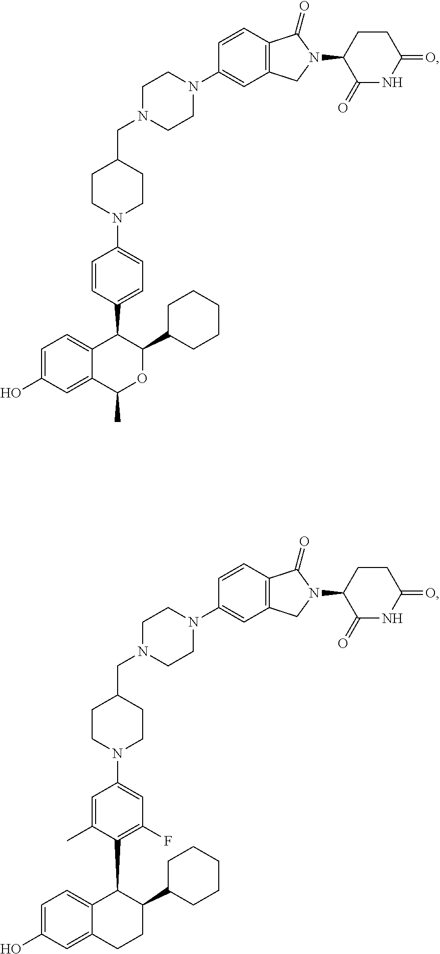 [SMILES: C[C@@H]1O[C@H](C2CCCCC2)[C@H](c2ccc(N3CCC(CN4CCN(c5ccc6c(c5)CN([C@H]5CCC(=O)NC5=O)C6=O)CC4)CC3)cc2)c2ccc(O)cc21 ; Cc1cc(N2CCC(CN3CCN(c4ccc5c(c4)CN([C@H]4CCC(=O)NC4=O)C5=O)CC3)CC2)cc(F)c1[C@@H]1c2ccc(O)cc2CC[C@@H]1C1CCCCC1] 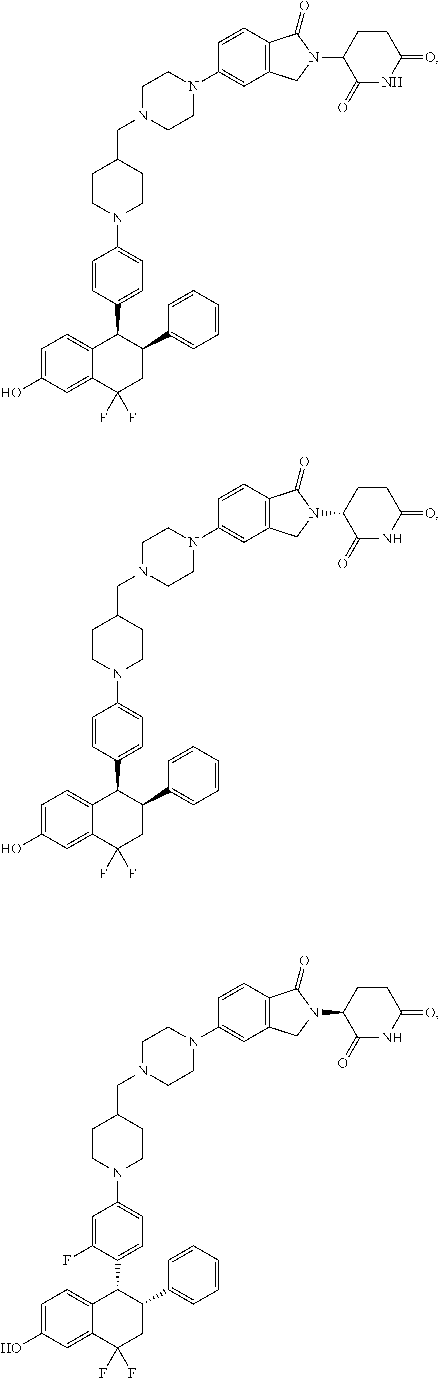 [SMILES: O=C1CCC(N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8C(F)(F)C[C@@H]7c7ccccc7)cc6)CC5)CC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccccc7)c(F)c6)CC5)CC4)ccc3C2=O)C(=O)N1] 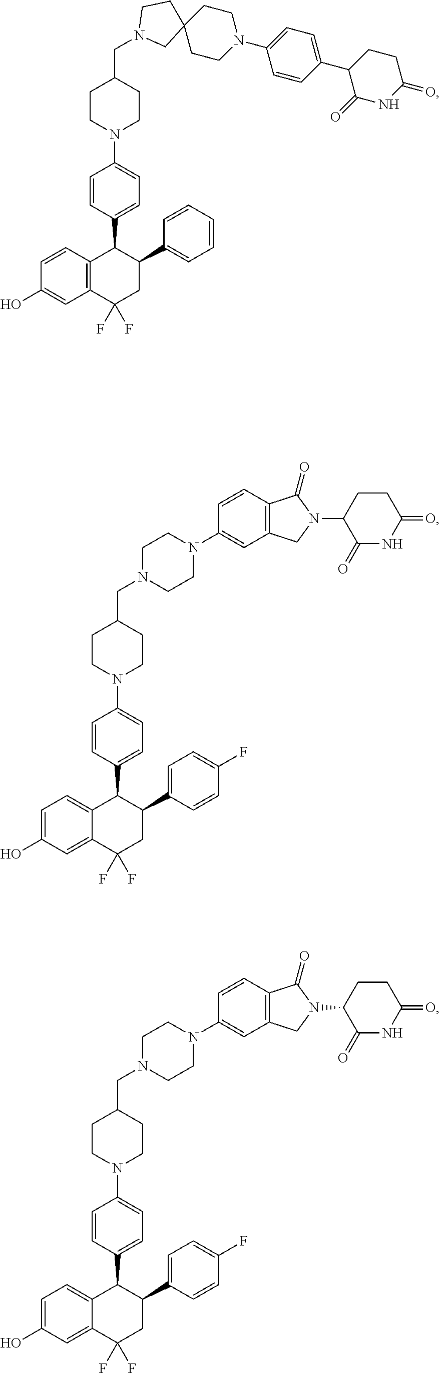 [SMILES: O=C1CCC(c2ccc(N3CCC4(CCN(CC5CCN(c6ccc([C@@H]7c8ccc(O)cc8C(F)(F)C[C@@H]7c7ccccc7)cc6)CC5)C4)CC3)cc2)C(=O)N1 ; O=C1CCC(N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8C(F)(F)C[C@@H]7c7ccc(F)cc7)cc6)CC5)CC4)ccc3C2=O)C(=O)N1] 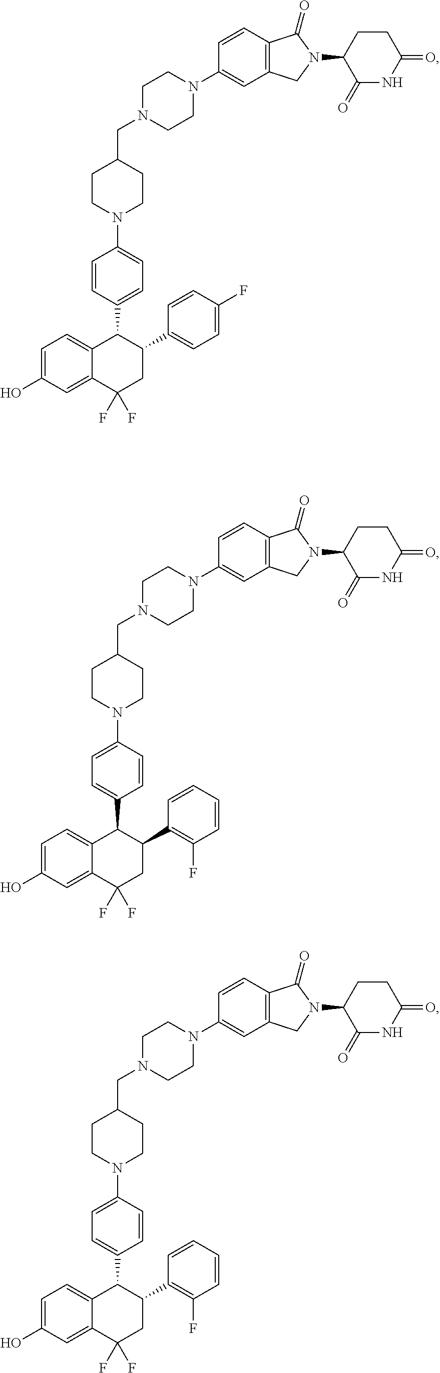 [SMILES: O=C1CC[C@H](N2Cc3cc(N4CCN(CC5CCN(c6ccc([C@H]7c8ccc(O)cc8C(F)(F)C[C@H]7c7ccc(F)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccccc7F)cc6)CC5)CC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccccc7F)cc6)CC5)CC4)ccc3C2=O)C(=O)N1] 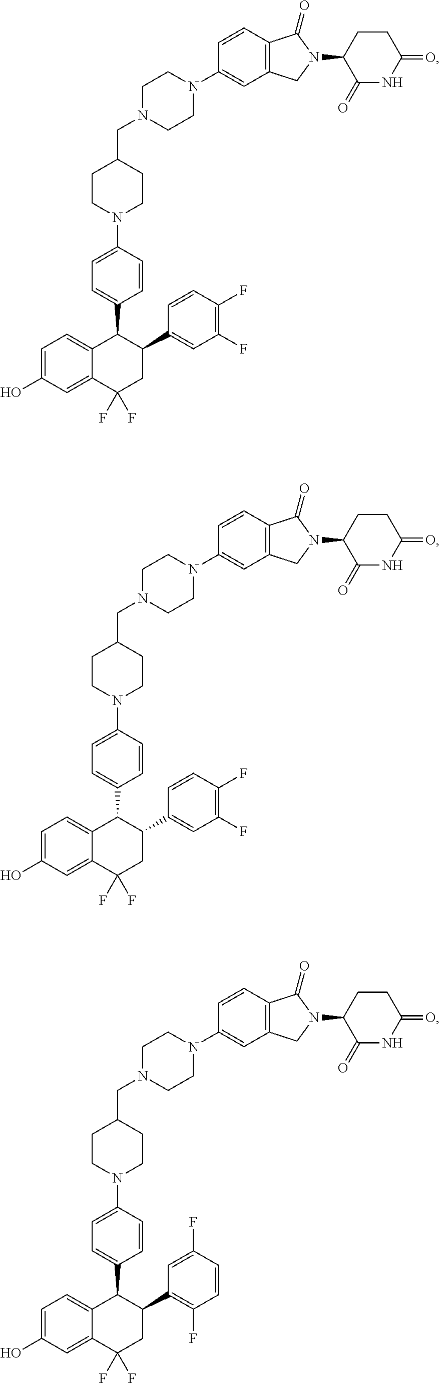 [SMILES: O=C1CC[C@H](N2Cc3cc(N4CCN(CC5CCN(c6ccc([C@@H]7c8ccc(O)cc8C(F)(F)C[C@@H]7c7ccc(F)c(F)c7)cc6)CC5)CC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccc(F)c(F)c7)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7cc(F)ccc7F)cc6)CC5)CC4)ccc3C2=O)C(=O)N1] 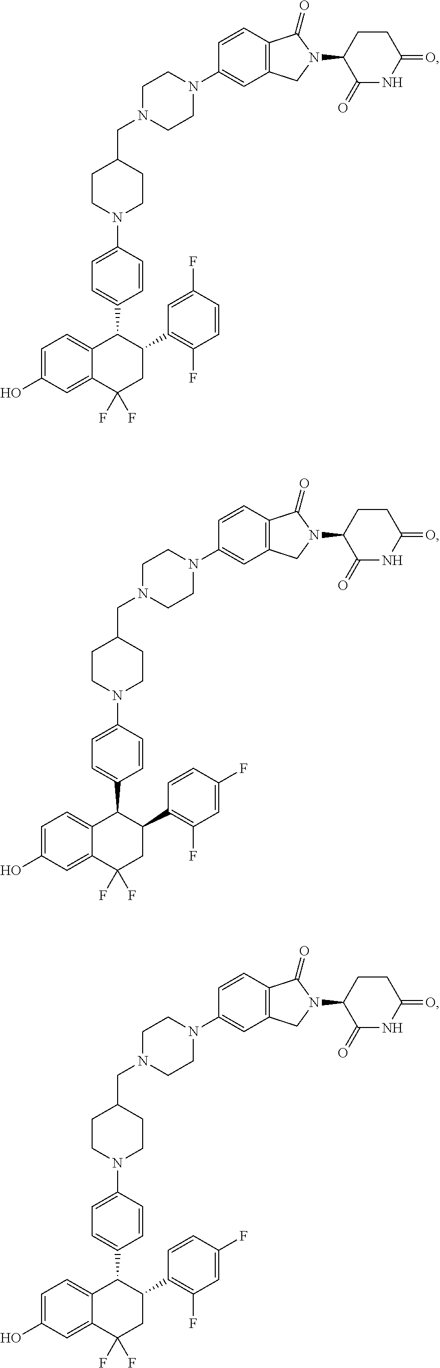 [SMILES: O=C1CC[C@H](N2Cc3cc(N4CCN(CC5CCN(c6ccc([C@H]7c8ccc(O)cc8C(F)(F)C[C@H]7c7cc(F)ccc7F)cc6)CC5)CC4)ccc3C2=O)C(=O)N1 ; O=C1CC[C@H](N2Cc3cc(N4CCN(CC5CCN(c6ccc([C@@H]7c8ccc(O)cc8C(F)(F)C[C@@H]7c7ccc(F)cc7F)cc6)CC5)CC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccc(F)cc7F)cc6)CC5)CC4)ccc3C2=O)C(=O)N1] 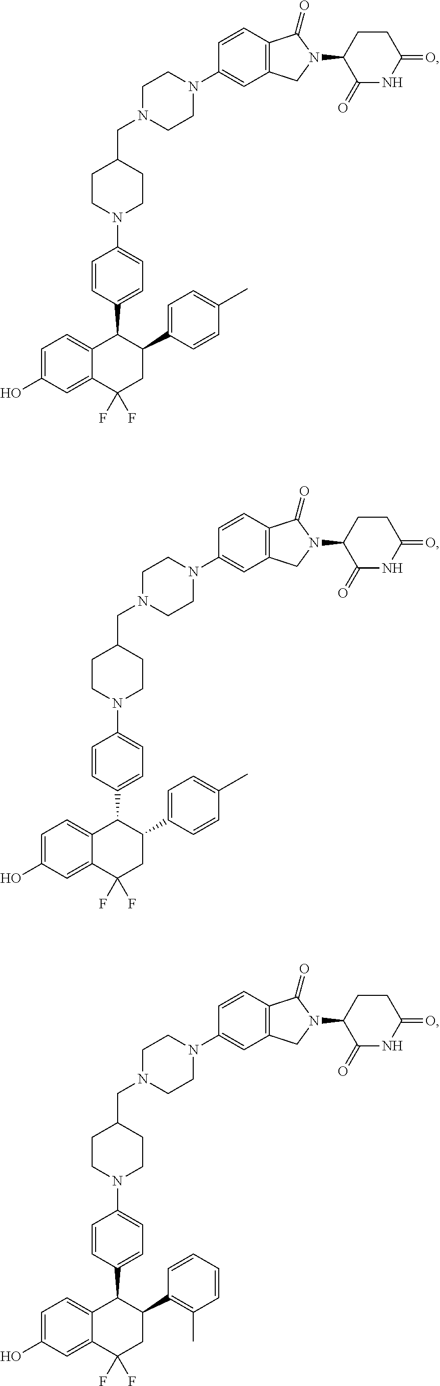 [SMILES: Cc1ccc([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)cc1 ; Cc1ccc([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)cc1 ; Cc1ccccc1[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1] 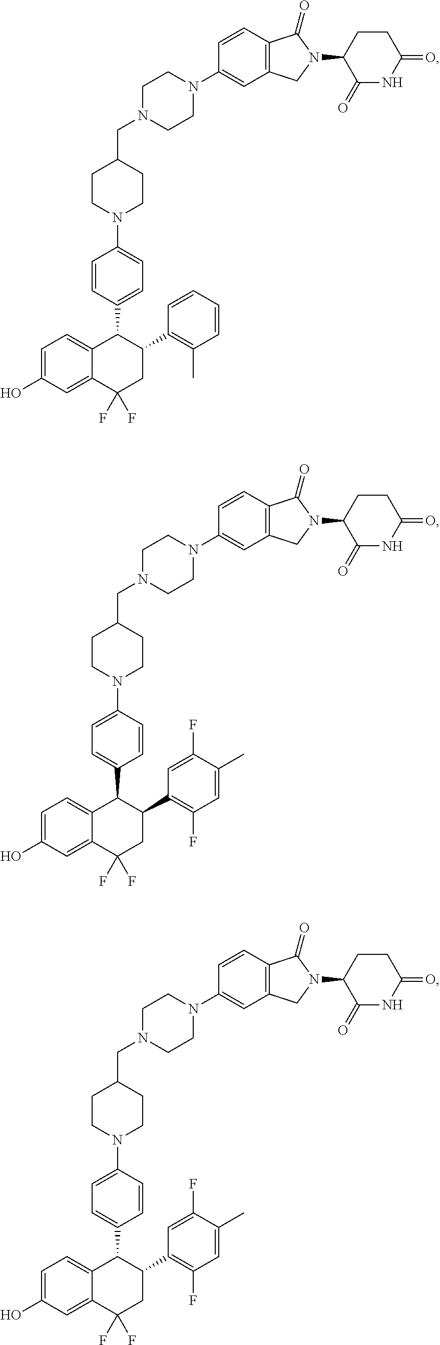 [SMILES: Cc1ccccc1[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1 ; Cc1cc(F)c([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)cc1F ; Cc1cc(F)c([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] 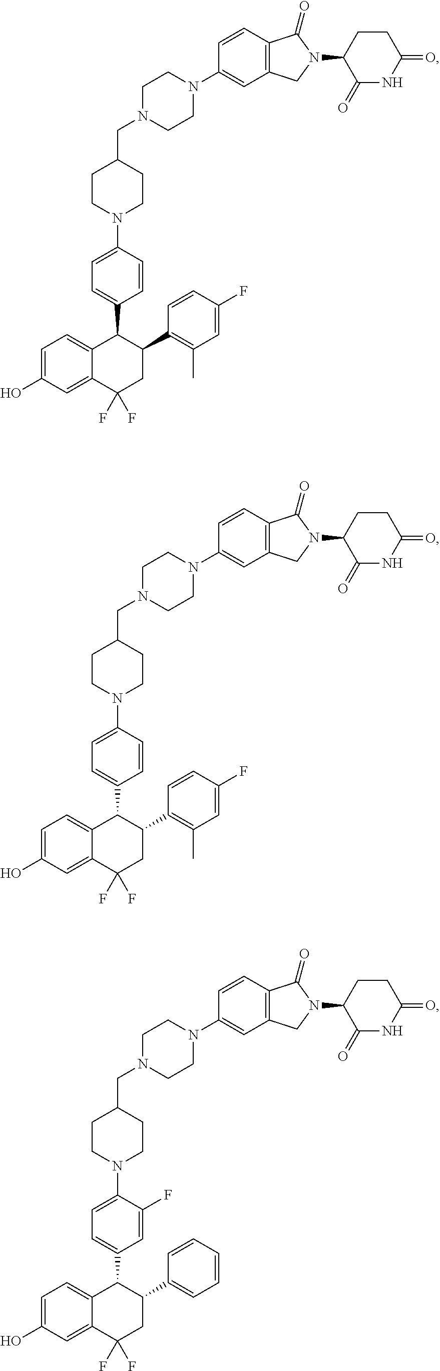 [SMILES: Cc1cc(F)ccc1[C@H]1CC(F)(F)c2cc(O)ccc2[C@H]1c1ccc(N2CCC(CN3CCN(c4ccc5c(c4)CN([C@H]4CCC(=O)NC4=O)C5=O)CC3)CC2)cc1 ; Cc1cc(F)ccc1[C@@H]1CC(F)(F)c2cc(O)ccc2[C@@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@H]7c8ccc(O)cc8C(F)(F)C[C@H]7c7ccccc7)cc6F)CC5)CC4)ccc3C2=O)C(=O)N1] 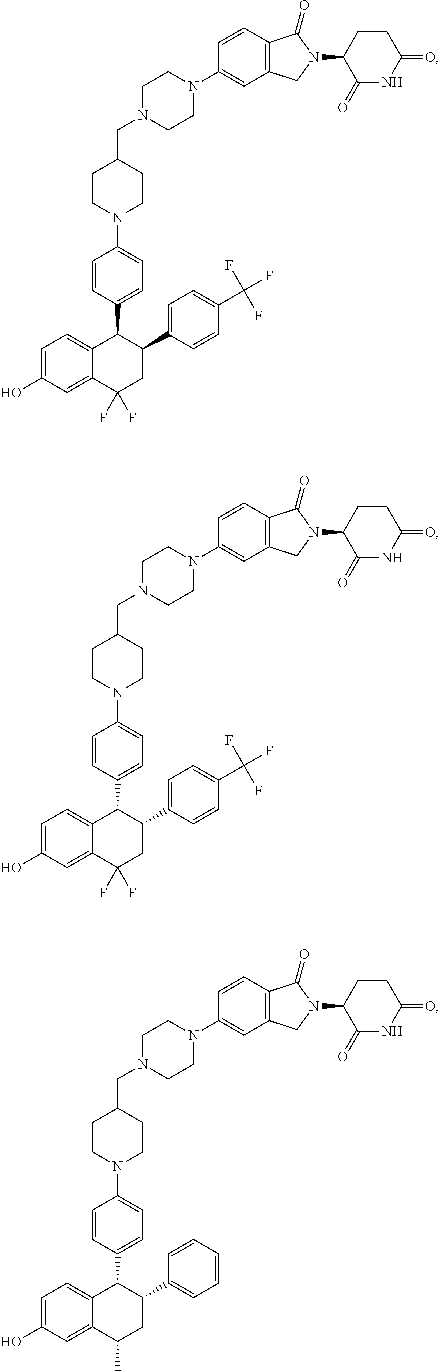 [SMILES: O=C1CC[C@H](N2Cc3cc(N4CCN(CC5CCN(c6ccc([C@@H]7c8ccc(O)cc8C(F)(F)C[C@@H]7c7ccc(C(F)(F)F)cc7)cc6)CC5)CC4)ccc3C2=O)C(=O)N1 ; O=C1CC[C@H](N2Cc3cc(N4CCN(CC5CCN(c6ccc([C@H]7c8ccc(O)cc8C(F)(F)C[C@H]7c7ccc(C(F)(F)F)cc7)cc6)CC5)CC4)ccc3C2=O)C(=O)N1 ; C[C@H]1C[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21] 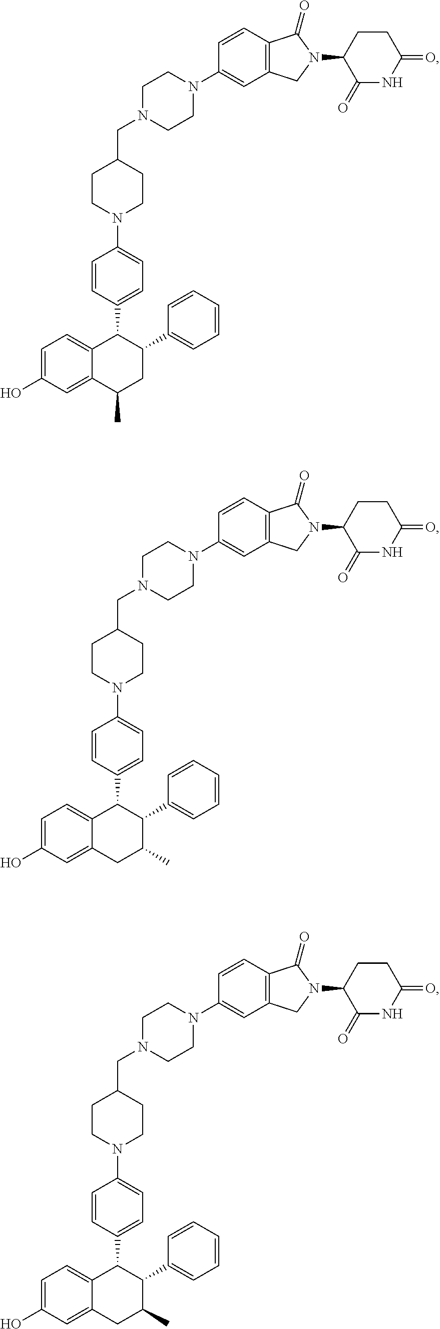 [SMILES: C[C@@H]1C[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21 ; C[C@@H]1Cc2cc(O)ccc2[C@H](c2ccc(N3CCC(CN4CCN(c5ccc6c(c5)CN([C@H]5CCC(=O)NC5=O)C6=O)CC4)CC3)cc2)[C@@H]1c1ccccc1 ; C[C@H]1Cc2cc(O)ccc2[C@H](c2ccc(N3CCC(CN4CCN(c5ccc6c(c5)CN([C@H]5CCC(=O)NC5=O)C6=O)CC4)CC3)cc2)[C@@H]1c1ccccc1] 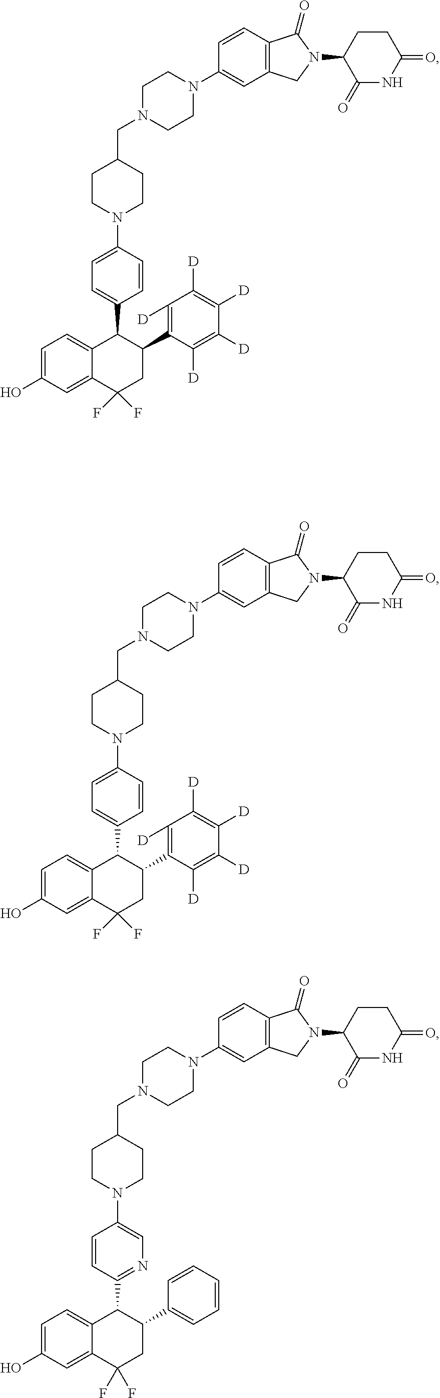 [SMILES: [2H]c1c([2H])c([2H])c([C@H]2CC(F)(F)c3cc(O)ccc3[C@H]2c2ccc(N3CCC(CN4CCN(c5ccc6c(c5)CN([C@H]5CCC(=O)NC5=O)C6=O)CC4)CC3)cc2)c([2H])c1[2H] ; [2H]c1c([2H])c([2H])c([C@@H]2CC(F)(F)c3cc(O)ccc3[C@@H]2c2ccc(N3CCC(CN4CCN(c5ccc6c(c5)CN([C@H]5CCC(=O)NC5=O)C6=O)CC4)CC3)cc2)c([2H])c1[2H] ; O=C1CC[C@H](N2Cc3cc(N4CCN(CC5CCN(c6ccc([C@H]7c8ccc(O)cc8C(F)(F)C[C@H]7c7ccccc7)nc6)CC5)CC4)ccc3C2=O)C(=O)N1] 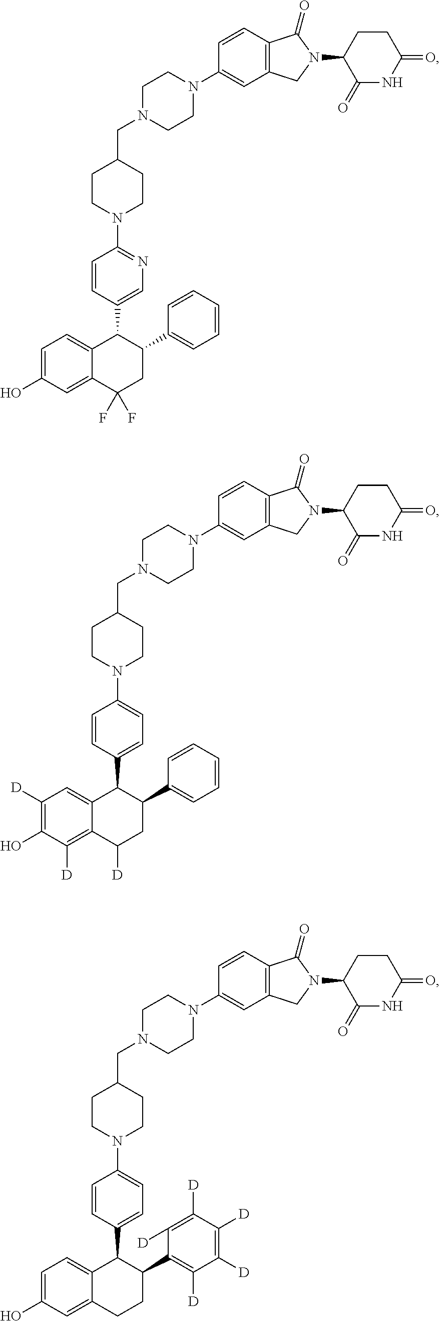 [SMILES: O=C1CC[C@H](N2Cc3cc(N4CCN(CC5CCN(c6ccc([C@H]7c8ccc(O)cc8C(F)(F)C[C@H]7c7ccccc7)cn6)CC5)CC4)ccc3C2=O)C(=O)N1 ; [2H]c1cc2c(c([2H])c1O)C([2H])C[C@H](c1ccccc1)[C@@H]2c1ccc(N2CCC(CN3CCN(c4ccc5c(c4)CN([C@H]4CCC(=O)NC4=O)C5=O)CC3)CC2)cc1 ; [2H]c1c([2H])c([2H])c([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)c([2H])c1[2H]] 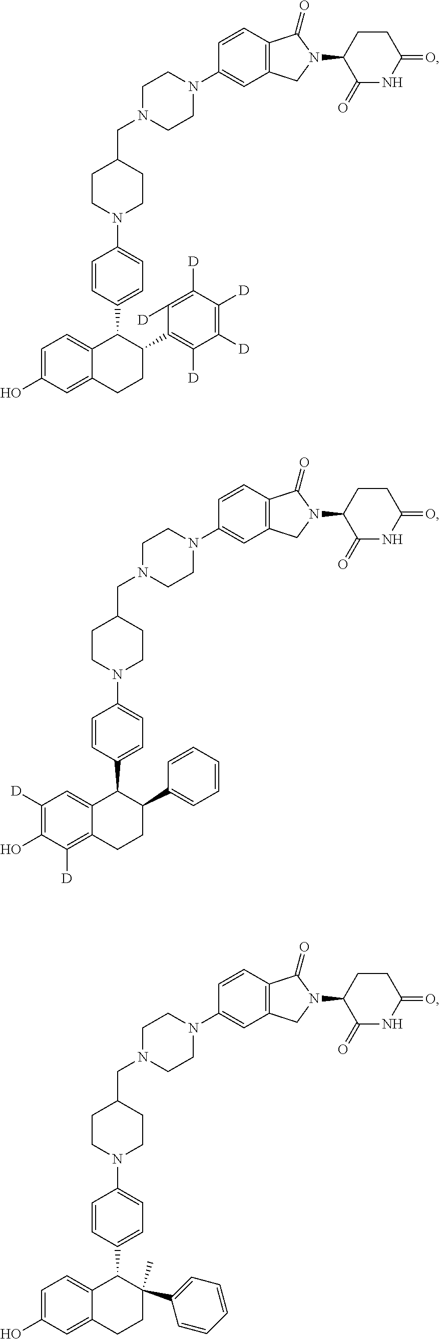 [SMILES: [2H]c1c([2H])c([2H])c([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)c([2H])c1[2H] ; [2H]c1cc2c(c([2H])c1O)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1 ; C[C@]1(c2ccccc2)CCc2cc(O)ccc2[C@@H]1c1ccc(N2CCC(CN3CCN(c4ccc5c(c4)CN([C@H]4CCC(=O)NC4=O)C5=O)CC3)CC2)cc1] 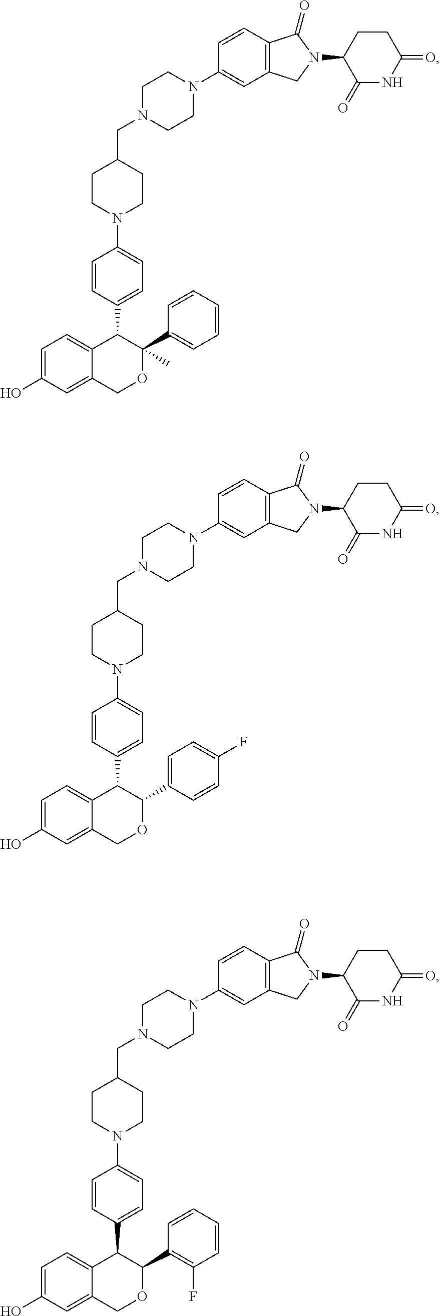 [SMILES: C[C@]1(c2ccccc2)OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@H]7c8ccc(O)cc8CO[C@H]7c7ccc(F)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccccc7F)cc6)CC5)CC4)ccc3C2=O)C(=O)N1] 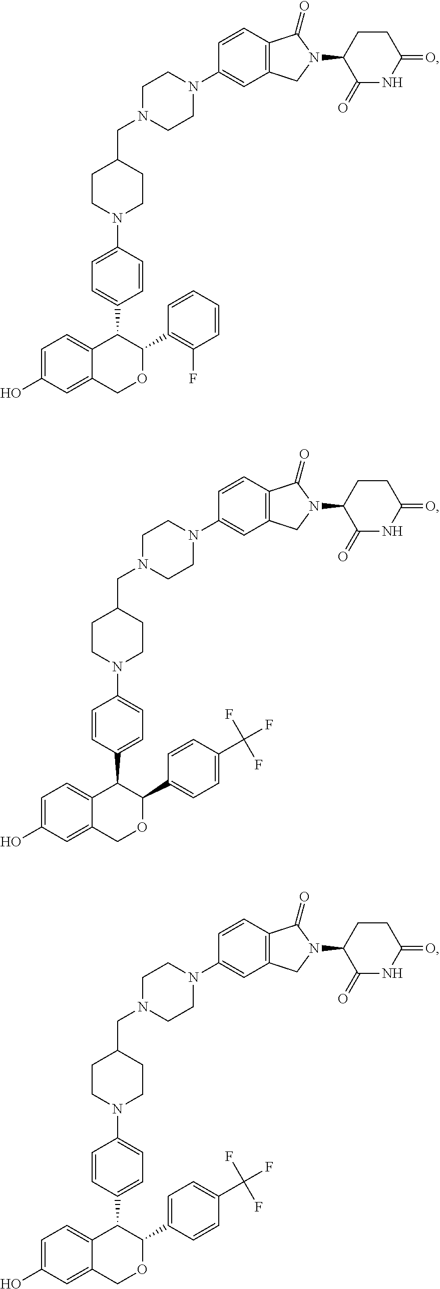 [SMILES: O=C1CC[C@H](N2Cc3cc(N4CCN(CC5CCN(c6ccc([C@H]7c8ccc(O)cc8CO[C@H]7c7ccccc7F)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccc(C(F)(F)F)cc7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7c7ccc(C(F)(F)F)cc7)cc6)CC5)CC4)ccc3C2=O)C(=O)N1] 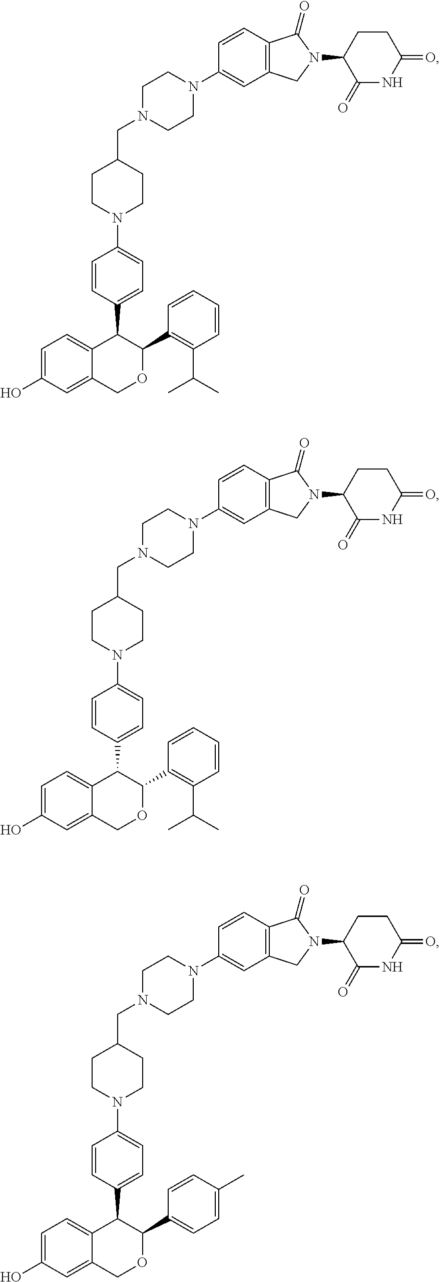 [SMILES: CC(C)c1ccccc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1 ; CC(C)c1ccccc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; Cc1ccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1] 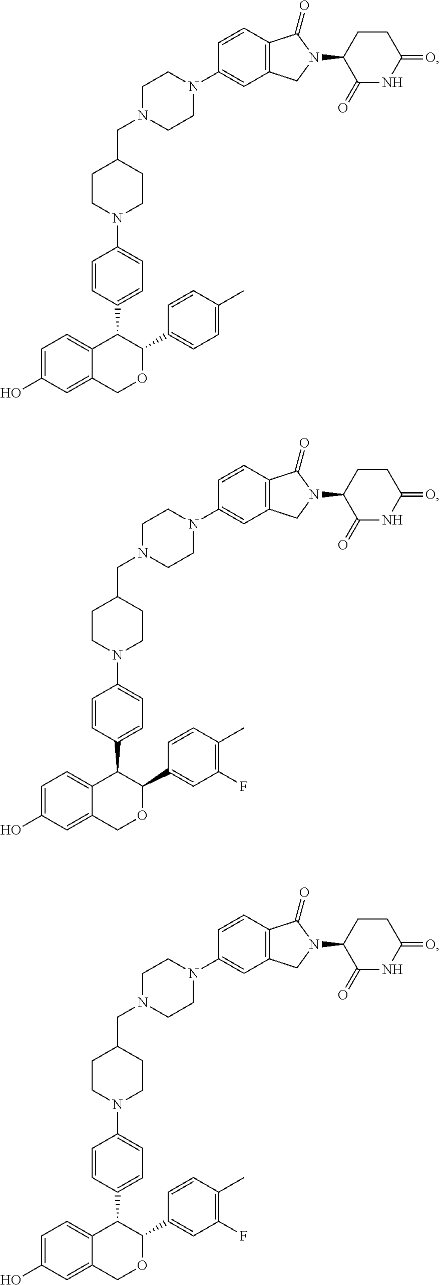 [SMILES: Cc1ccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1 ; Cc1ccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F ; Cc1ccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] 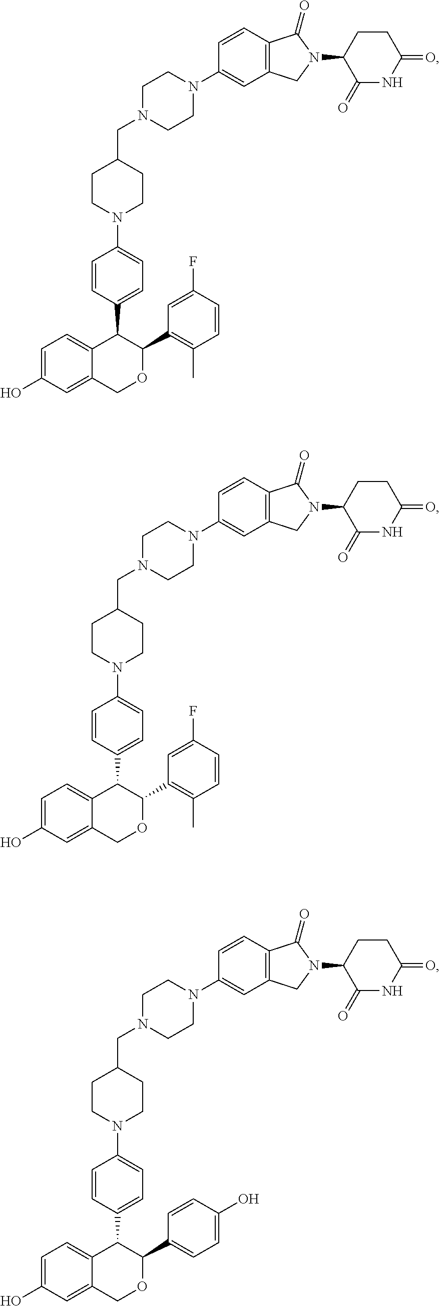 [SMILES: Cc1ccc(F)cc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1 ; Cc1ccc(F)cc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; O=C1CC[C@H](N2Cc3cc(N4CCN(CC5CCN(c6ccc([C@H]7c8ccc(O)cc8CO[C@@H]7c7ccc(O)cc7)cc6)CC5)CC4)ccc3C2=O)C(=O)N1] 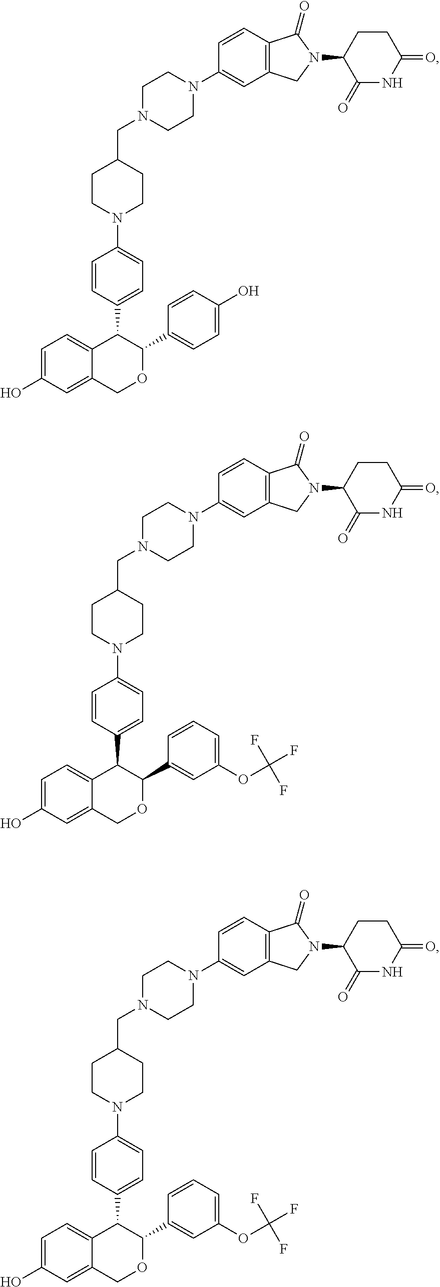 [SMILES: O=C1CC[C@H](N2Cc3cc(N4CCN(CC5CCN(c6ccc([C@H]7c8ccc(O)cc8CO[C@H]7c7ccc(O)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7cccc(OC(F)(F)F)c7)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7c7cccc(OC(F)(F)F)c7)cc6)CC5)CC4)ccc3C2=O)C(=O)N1] 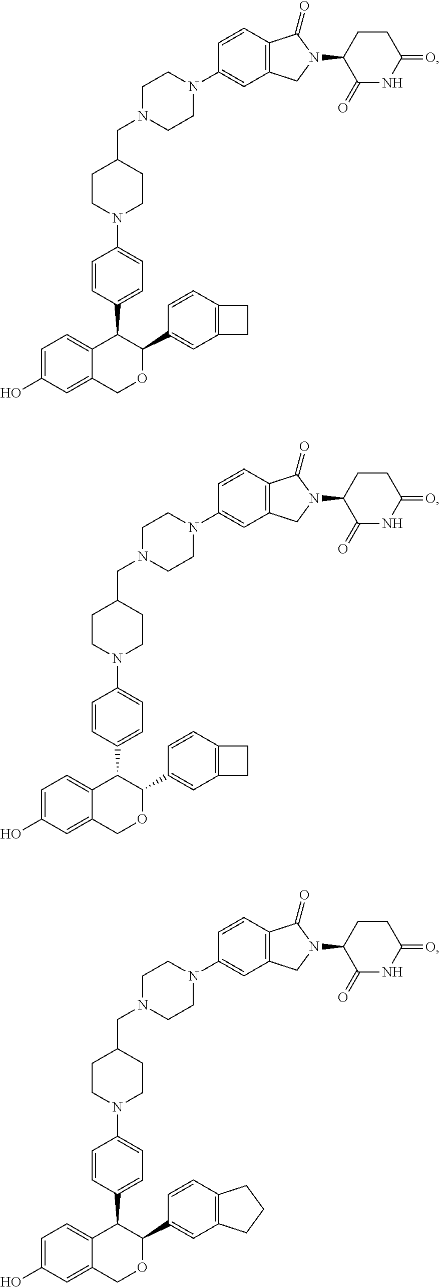 [SMILES: O=C1CC[C@H](N2Cc3cc(N4CCN(CC5CCN(c6ccc([C@@H]7c8ccc(O)cc8CO[C@@H]7c7ccc8c(c7)CC8)cc6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7c7ccc8c(c7)CC8)cc6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccc8c(c7)CCC8)cc6)CC5)CC4)ccc3C2=O)C(=O)N1] 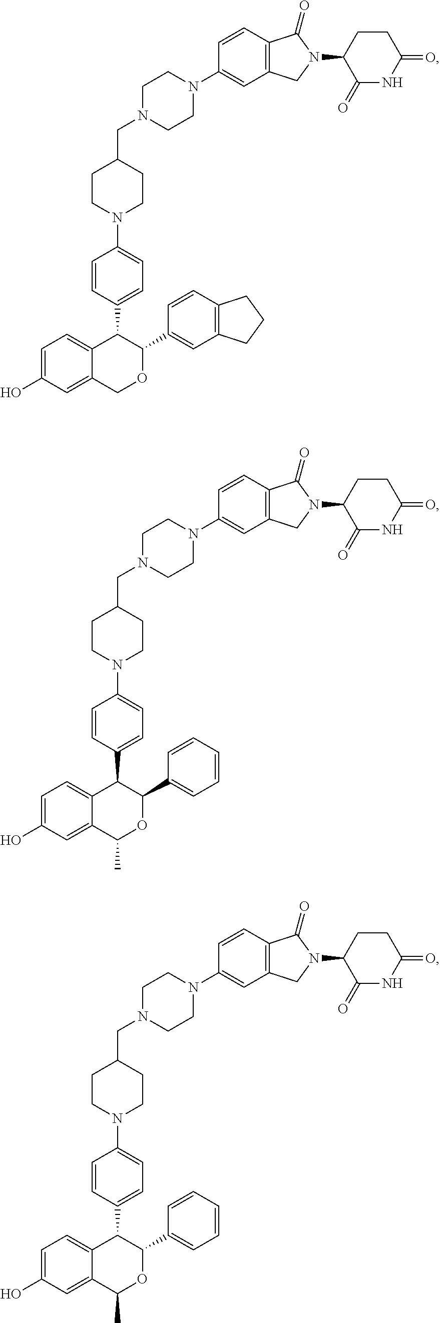 [SMILES: O=C1CC[C@H](N2Cc3cc(N4CCN(CC5CCN(c6ccc([C@H]7c8ccc(O)cc8CO[C@H]7c7ccc8c(c7)CCC8)cc6)CC5)CC4)ccc3C2=O)C(=O)N1 ; C[C@H]1O[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21 ; C[C@@H]1O[C@@H](c2ccccc2)[C@@H](c2ccc(N3CCC(CN4CCN(c5ccc6c(c5)CN([C@H]5CCC(=O)NC5=O)C6=O)CC4)CC3)cc2)c2ccc(O)cc21] 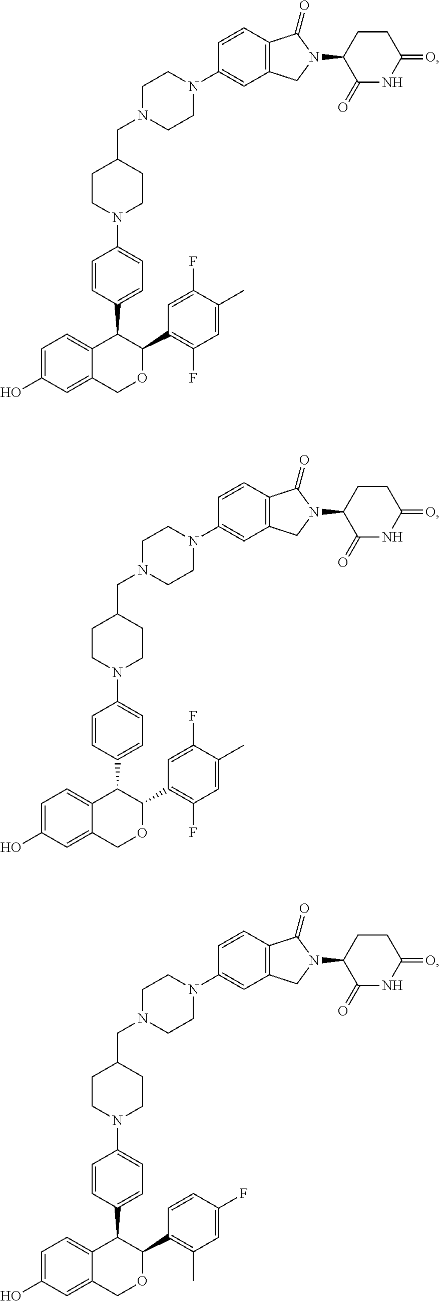 [SMILES: Cc1cc(F)c([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F ; Cc1cc(F)c([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F ; Cc1cc(F)ccc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1] 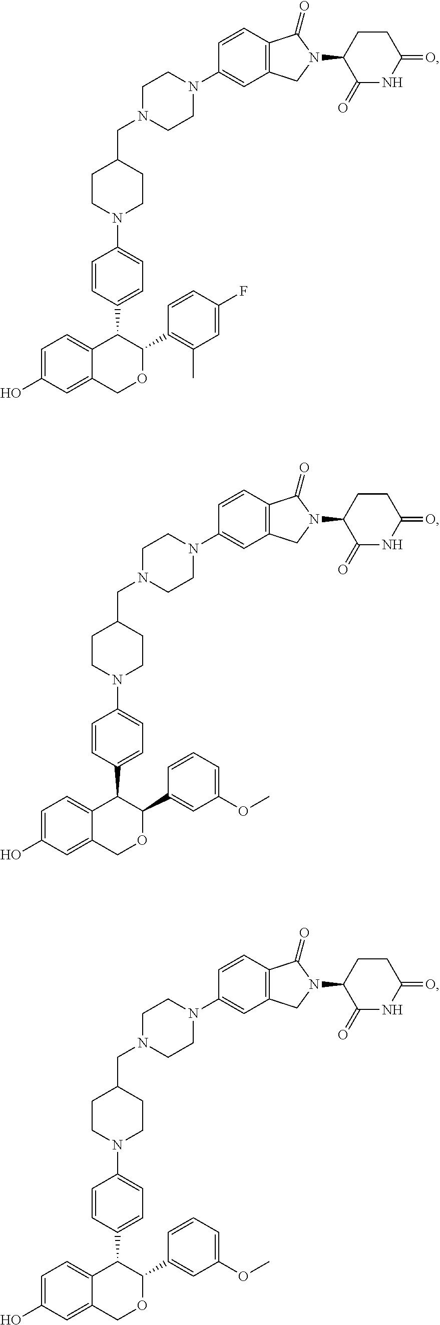 [SMILES: Cc1cc(F)ccc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1 ; COc1cccc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)c1 ; COc1cccc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)c1] 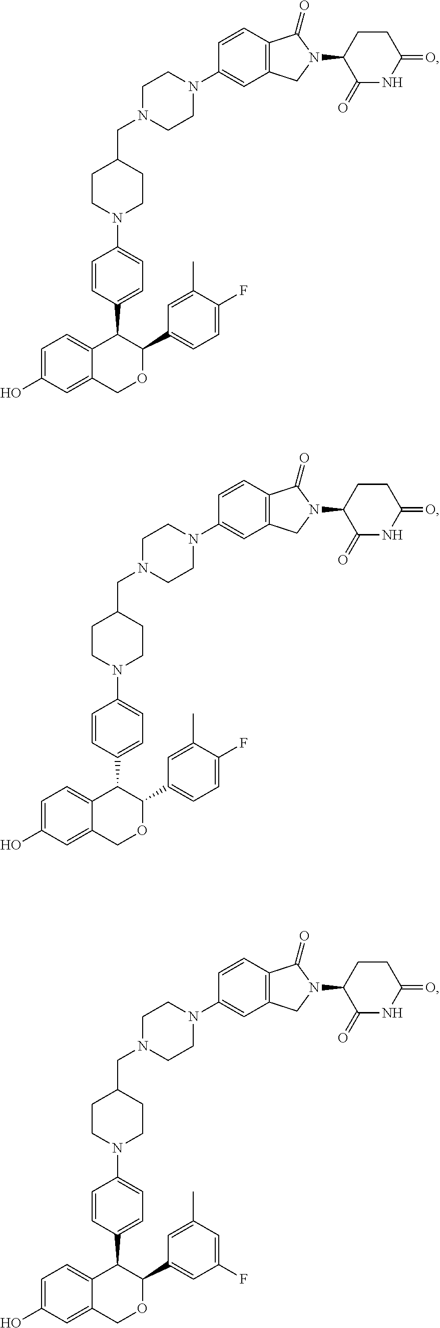 [SMILES: Cc1cc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)ccc1F ; Cc1cc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)ccc1F ; Cc1cc(F)cc([C@H]2OCc3cc(O)ccc3[C@H]2c2ccc(N3CCC(CN4CCN(c5ccc6c(c5)CN([C@H]5CCC(=O)NC5=O)C6=O)CC4)CC3)cc2)c1] 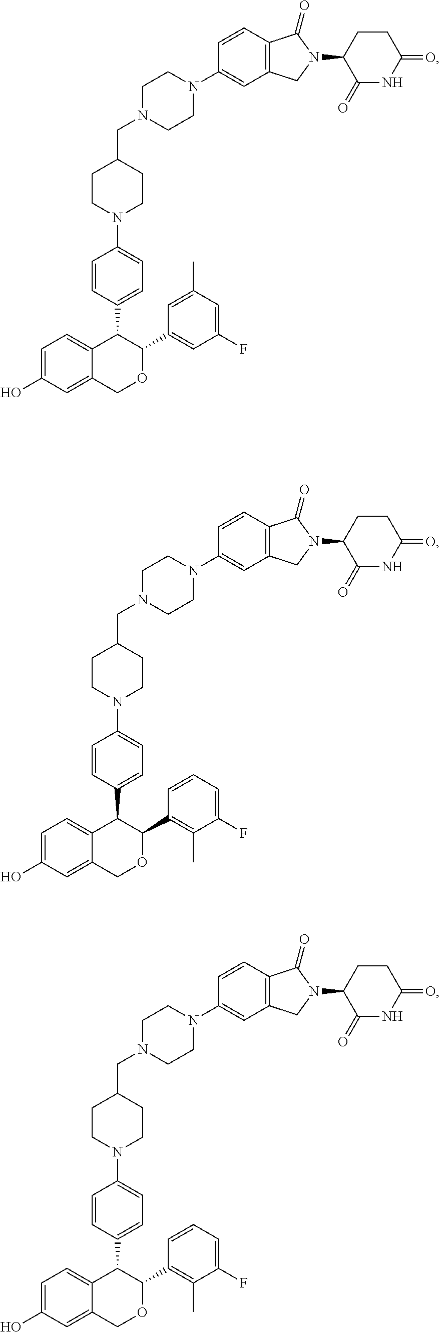 [SMILES: Cc1cc(F)cc([C@@H]2OCc3cc(O)ccc3[C@@H]2c2ccc(N3CCC(CN4CCN(c5ccc6c(c5)CN([C@H]5CCC(=O)NC5=O)C6=O)CC4)CC3)cc2)c1 ; Cc1c(F)cccc1[C@H]1OCc2cc(O)ccc2[C@H]1c1ccc(N2CCC(CN3CCN(c4ccc5c(c4)CN([C@H]4CCC(=O)NC4=O)C5=O)CC3)CC2)cc1 ; Cc1c(F)cccc1[C@@H]1OCc2cc(O)ccc2[C@@H]1c1ccc(N2CCC(CN3CCN(c4ccc5c(c4)CN([C@H]4CCC(=O)NC4=O)C5=O)CC3)CC2)cc1] 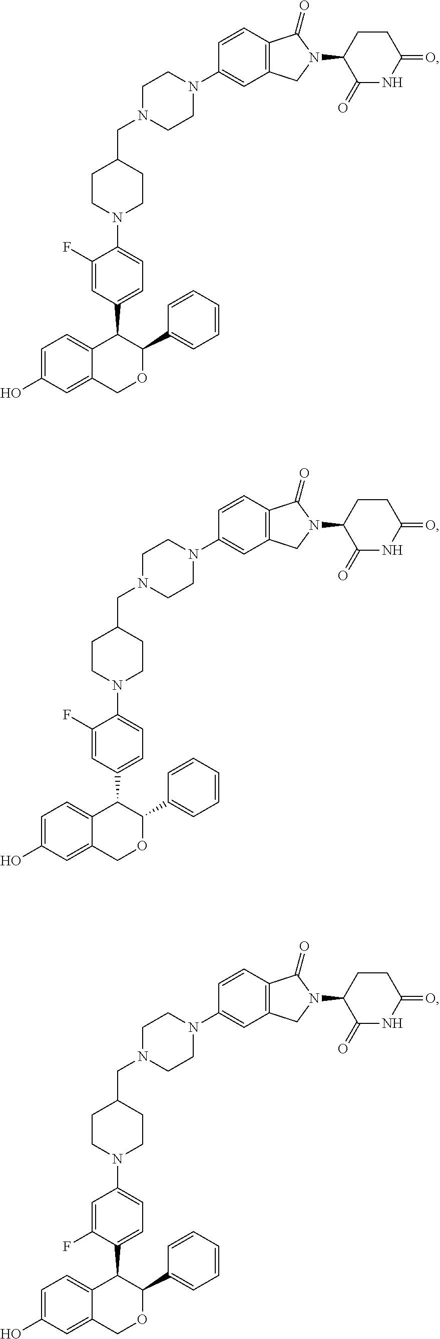 [SMILES: O=C1CC[C@H](N2Cc3cc(N4CCN(CC5CCN(c6ccc([C@@H]7c8ccc(O)cc8CO[C@@H]7c7ccccc7)cc6F)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7c7ccccc7)cc6F)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccccc7)c(F)c6)CC5)CC4)ccc3C2=O)C(=O)N1] 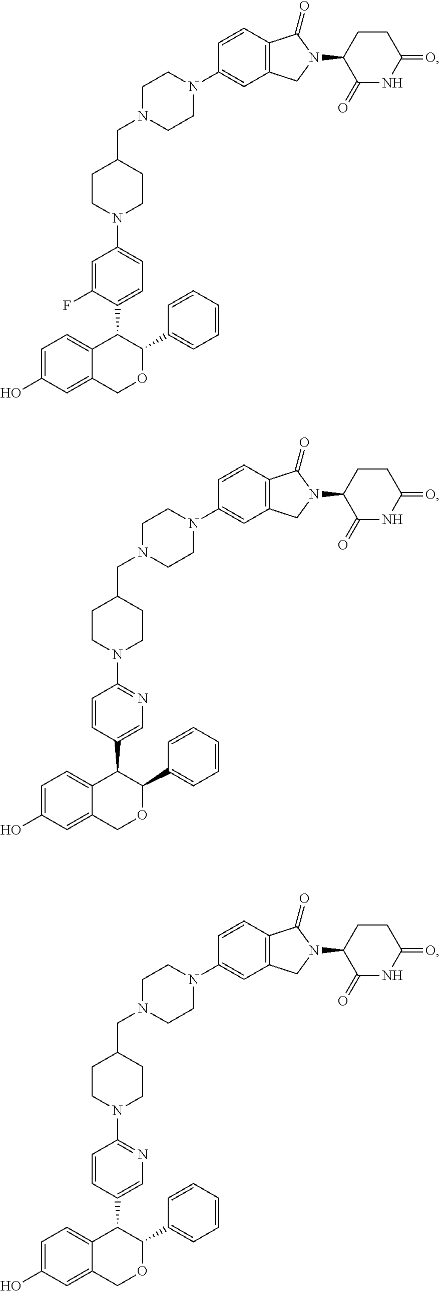 [SMILES: O=C1CC[C@H](N2Cc3cc(N4CCN(CC5CCN(c6ccc([C@H]7c8ccc(O)cc8CO[C@H]7c7ccccc7)c(F)c6)CC5)CC4)ccc3C2=O)C(=O)N1 ; O=C1CC[C@H](N2Cc3cc(N4CCN(CC5CCN(c6ccc([C@@H]7c8ccc(O)cc8CO[C@@H]7c7ccccc7)cn6)CC5)CC4)ccc3C2=O)C(=O)N1 ; O=C1CC[C@H](N2Cc3cc(N4CCN(CC5CCN(c6ccc([C@H]7c8ccc(O)cc8CO[C@H]7c7ccccc7)cn6)CC5)CC4)ccc3C2=O)C(=O)N1] 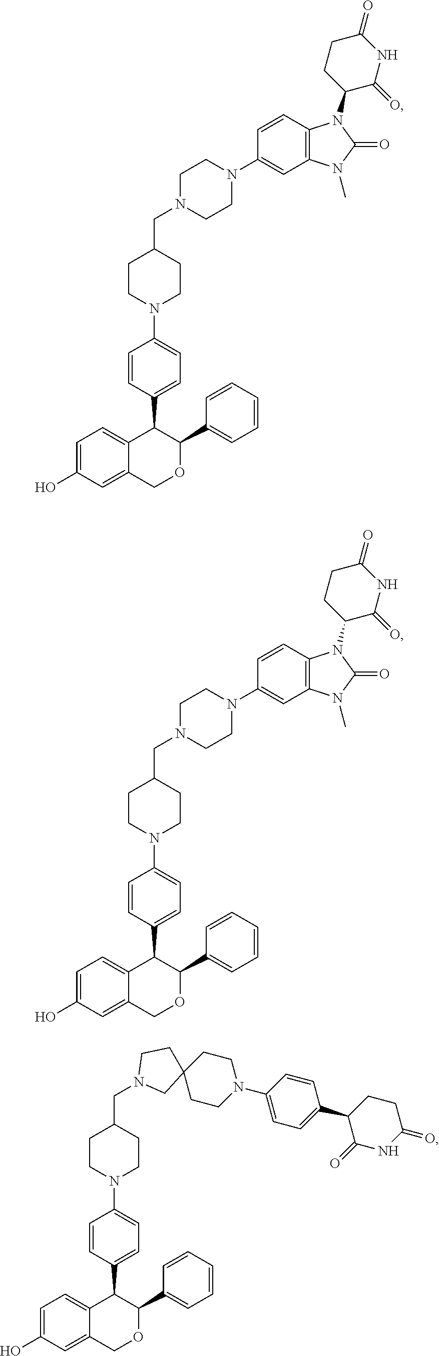 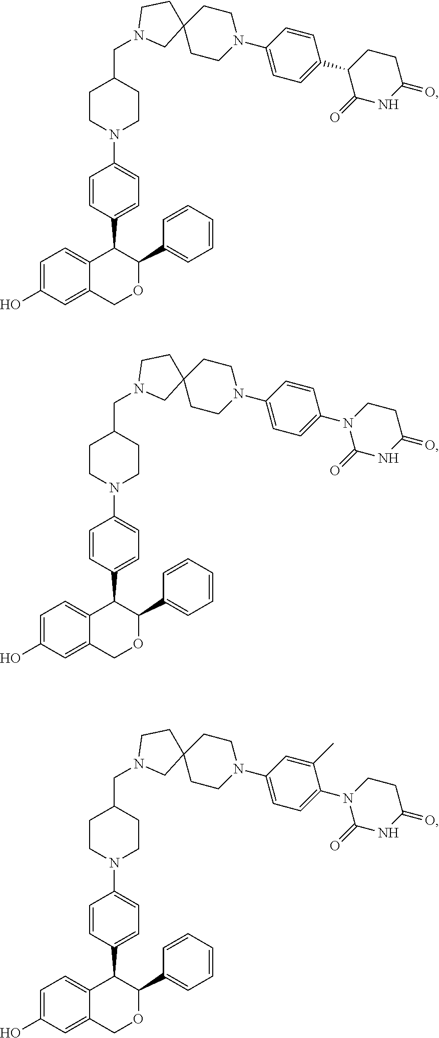 [SMILES: O=C1CC[C@@H](c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1 ; O=C1CCN(c2ccc(N3CCC4(CCN(CC5CCN(c6ccc([C@@H]7c8ccc(O)cc8CO[C@@H]7c7ccccc7)cc6)CC5)C4)CC3)cc2)C(=O)N1 ; Cc1cc(N2CCC3(CCN(CC4CCN(c5ccc([C@@H]6c7ccc(O)cc7CO[C@@H]6c6ccccc6)cc5)CC4)C3)CC2)ccc1N1CCC(=O)NC1=O] 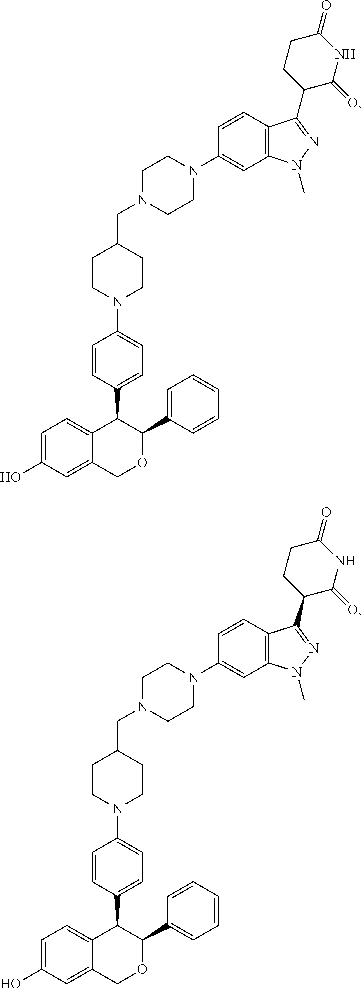 [SMILES: Cn1nc(C2CCC(=O)NC2=O)c2ccc(N3CCN(CC4CCN(c5ccc([C@@H]6c7ccc(O)cc7CO[C@@H]6c6ccccc6)cc5)CC4)CC3)cc21 ; Cn1nc([C@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21] 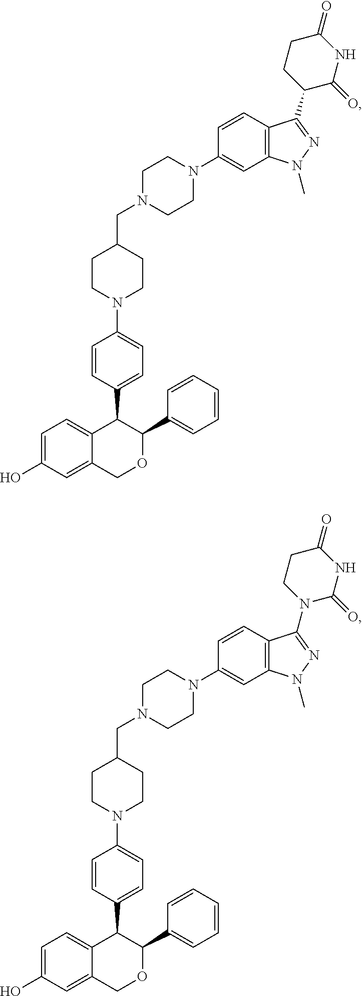 [SMILES: Cn1nc([C@@H]2CCC(=O)NC2=O)c2ccc(N3CCN(CC4CCN(c5ccc([C@@H]6c7ccc(O)cc7CO[C@@H]6c6ccccc6)cc5)CC4)CC3)cc21 ; Cn1nc(N2CCC(=O)NC2=O)c2ccc(N3CCN(CC4CCN(c5ccc([C@@H]6c7ccc(O)cc7CO[C@@H]6c6ccccc6)cc5)CC4)CC3)cc21] 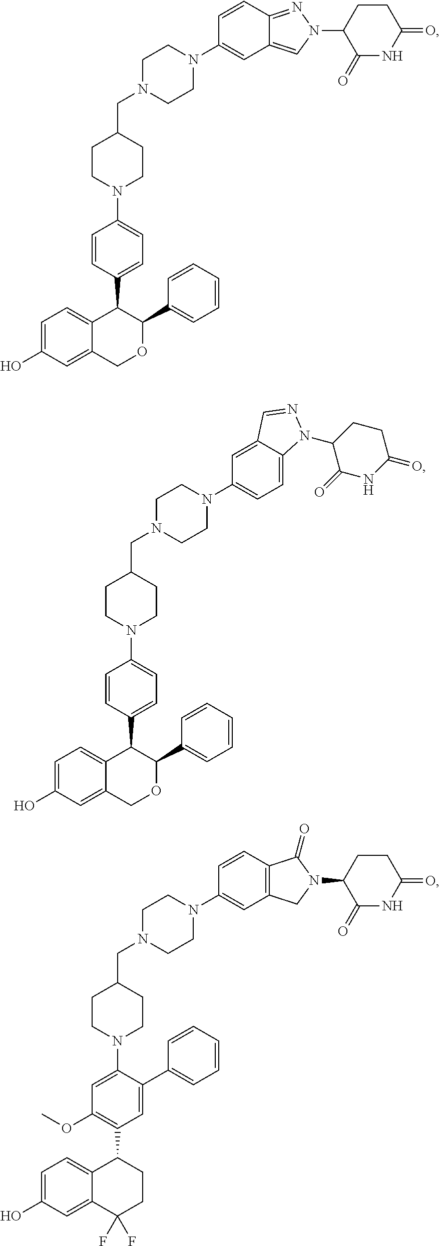 [SMILES: O=C1CCC(n2cc3cc(N4CCN(CC5CCN(c6ccc([C@@H]7c8ccc(O)cc8CO[C@@H]7c7ccccc7)cc6)CC5)CC4)ccc3n2)C(=O)N1 ; O=C1CCC(n2ncc3cc(N4CCN(CC5CCN(c6ccc([C@@H]7c8ccc(O)cc8CO[C@@H]7c7ccccc7)cc6)CC5)CC4)ccc32)C(=O)N1 ; COc1cc(N2CCC(CN3CCN(c4ccc5c(c4)CN([C@H]4CCC(=O)NC4=O)C5=O)CC3)CC2)c(-c2ccccc2)cc1[C@@H]1CCC(F)(F)c2cc(O)ccc21] 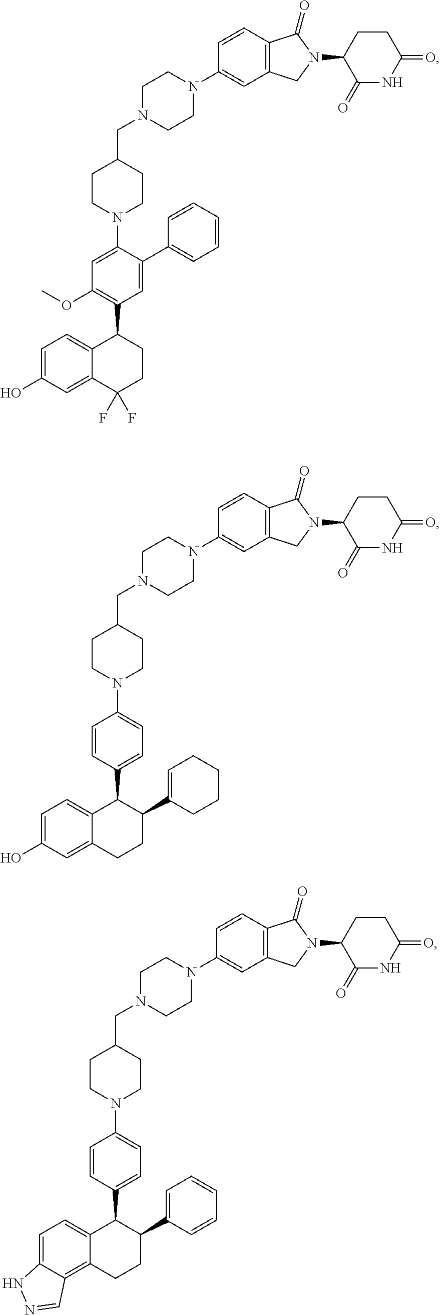 [SMILES: COc1cc(N2CCC(CN3CCN(c4ccc5c(c4)CN([C@H]4CCC(=O)NC4=O)C5=O)CC3)CC2)c(-c2ccccc2)cc1[C@H]1CCC(F)(F)c2cc(O)ccc21 ; O=C1CC[C@H](N2Cc3cc(N4CCN(CC5CCN(c6ccc([C@@H]7c8ccc(O)cc8CC[C@@H]7C7=CCCCC7)cc6)CC5)CC4)ccc3C2=O)C(=O)N1 ; O=C1CC[C@H](N2Cc3cc(N4CCN(CC5CCN(c6ccc([C@@H]7c8ccc9[nH]ncc9c8CC[C@@H]7c7ccccc7)cc6)CC5)CC4)ccc3C2=O)C(=O)N1] 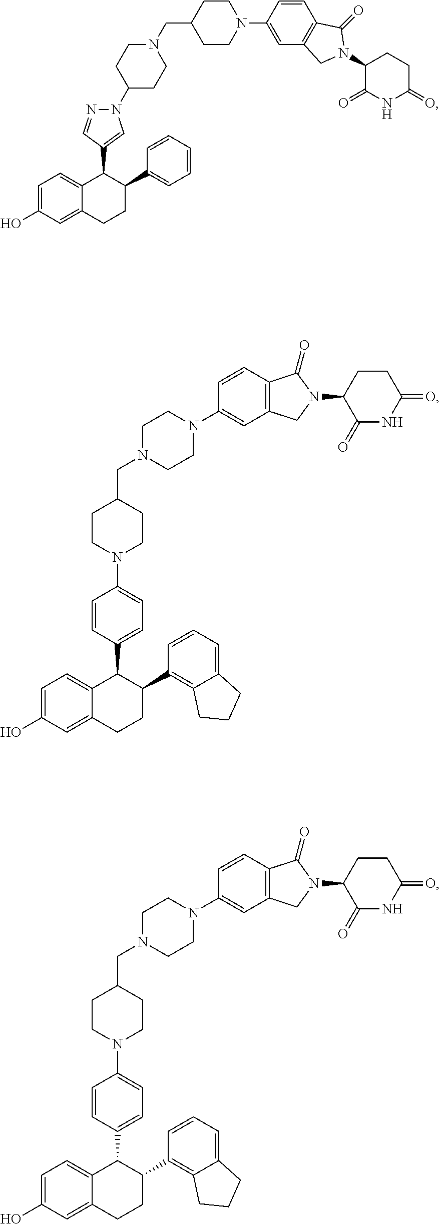 [SMILES: O=C1CC[C@H](N2Cc3cc(N4CCC(CN5CCC(n6cc([C@@H]7c8ccc(O)cc8CC[C@@H]7c7ccccc7)cn6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc8c7CCC8)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7cccc8c7CCC8)cc6)CC5)CC4)ccc3C2=O)C(=O)N1] 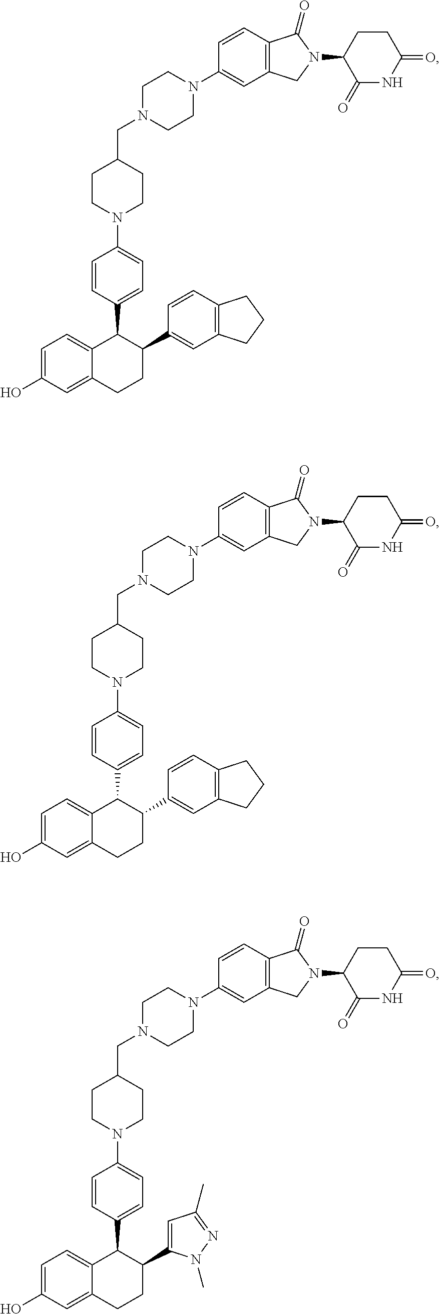 [SMILES: O=C1CC[C@H](N2Cc3cc(N4CCN(CC5CCN(c6ccc([C@@H]7c8ccc(O)cc8CC[C@@H]7c7ccc8c(c7)CCC8)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc8c(c7)CCC8)cc6)CC5)CC4)ccc3C2=O)C(=O)N1 ; Cc1cc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)n(C)n1] 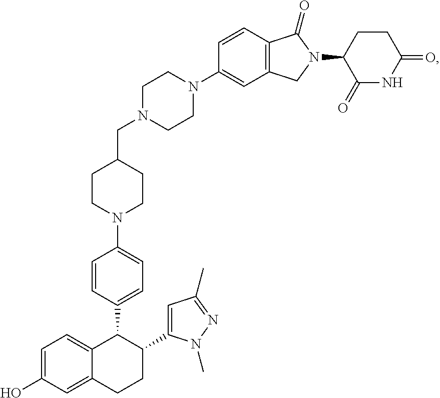 [SMILES: Cc1cc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)n(C)n1] 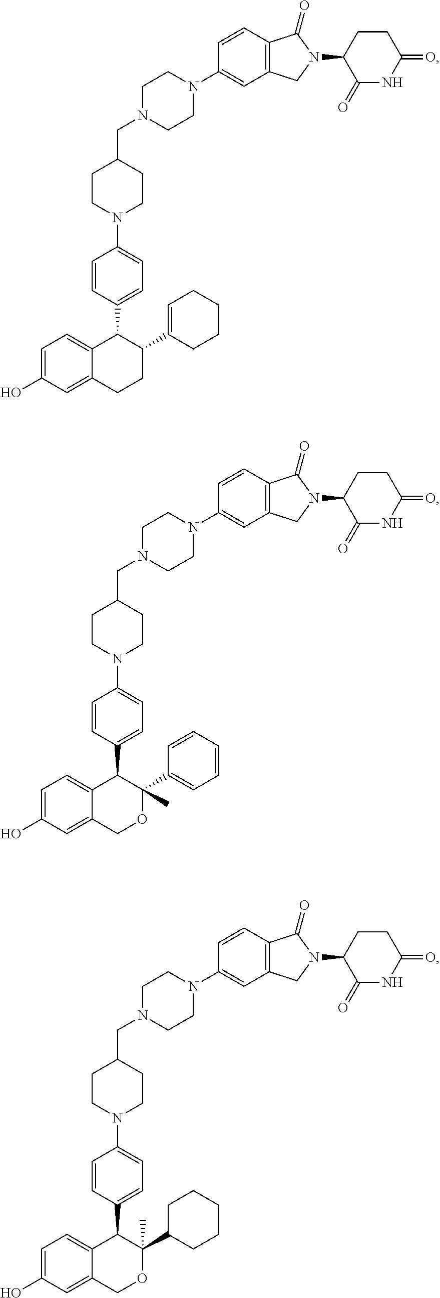 [SMILES: O=C1CC[C@H](N2Cc3cc(N4CCN(CC5CCN(c6ccc([C@H]7c8ccc(O)cc8CC[C@H]7C7=CCCCC7)cc6)CC5)CC4)ccc3C2=O)C(=O)N1 ; C[C@@]1(c2ccccc2)OCc2cc(O)ccc2[C@H]1c1ccc(N2CCC(CN3CCN(c4ccc5c(c4)CN([C@H]4CCC(=O)NC4=O)C5=O)CC3)CC2)cc1 ; C[C@]1(C2CCCCC2)OCc2cc(O)ccc2[C@H]1c1ccc(N2CCC(CN3CCN(c4ccc5c(c4)CN([C@H]4CCC(=O)NC4=O)C5=O)CC3)CC2)cc1] 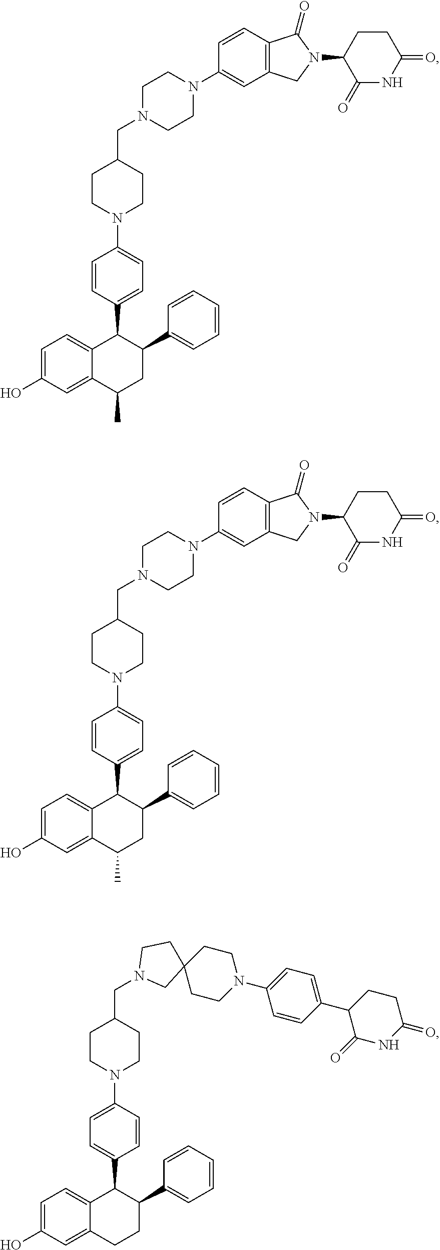 [SMILES: C[C@@H]1C[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21 ; C[C@H]1C[C@H](c2ccccc2)[C@H](c2ccc(N3CCC(CN4CCN(c5ccc6c(c5)CN([C@H]5CCC(=O)NC5=O)C6=O)CC4)CC3)cc2)c2ccc(O)cc21 ; O=C1CCC(c2ccc(N3CCC4(CCN(CC5CCN(c6ccc([C@@H]7c8ccc(O)cc8CC[C@@H]7c7ccccc7)cc6)CC5)C4)CC3)cc2)C(=O)N1] 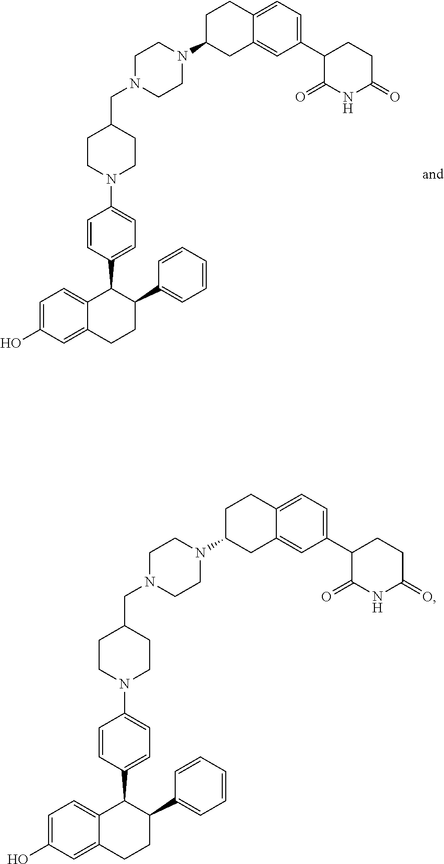 [SMILES: O=C1CCC(c2ccc3c(c2)C[C@@H](N2CCN(CC4CCN(c5ccc([C@@H]6c7ccc(O)cc7CC[C@@H]6c6ccccc6)cc5)CC4)CC2)CC3)C(=O)N1 ; O=C1CCC(c2ccc3c(c2)C[C@H](N2CCN(CC4CCN(c5ccc([C@@H]6c7ccc(O)cc7CC[C@@H]6c6ccccc6)cc5)CC4)CC2)CC3)C(=O)N1]
or a pharmaceutically acceptable salt thereof.

121. A compound selected from: 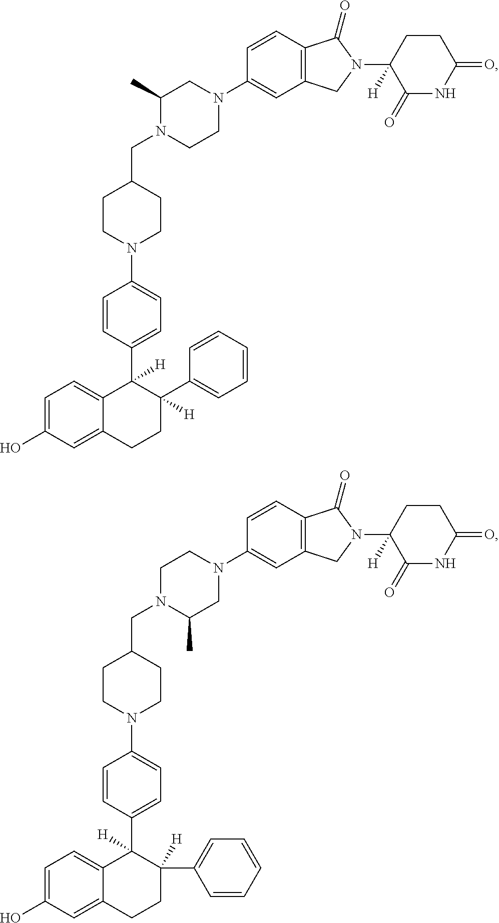 [SMILES: C[C@H]1CN(c2ccc3c(c2)CN([C@H]2CCC(=O)NC2=O)C3=O)CCN1CC1CCN(c2ccc([C@@H]3c4ccc(O)cc4CC[C@@H]3c3ccccc3)cc2)CC1 ; C[C@@H]1CN(c2ccc3c(c2)CN([C@H]2CCC(=O)NC2=O)C3=O)CCN1CC1CCN(c2ccc([C@@H]3c4ccc(O)cc4CC[C@@H]3c3ccccc3)cc2)CC1] 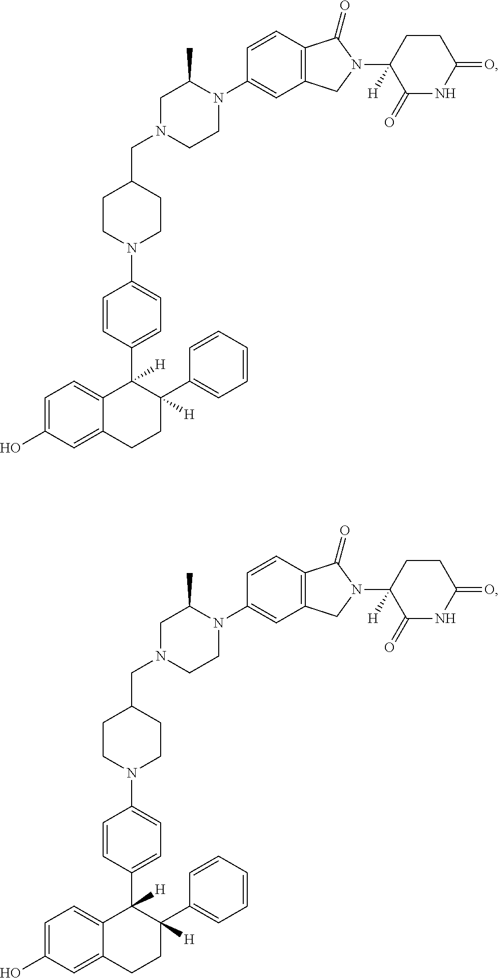 [SMILES: C[C@@H]1CN(CC2CCN(c3ccc([C@@H]4c5ccc(O)cc5CC[C@@H]4c4ccccc4)cc3)CC2)CCN1c1ccc2c(c1)CN([C@H]1CCC(=O)NC1=O)C2=O ; C[C@@H]1CN(CC2CCN(c3ccc([C@H]4c5ccc(O)cc5CC[C@H]4c4ccccc4)cc3)CC2)CCN1c1ccc2c(c1)CN([C@H]1CCC(=O)NC1=O)C2=O] 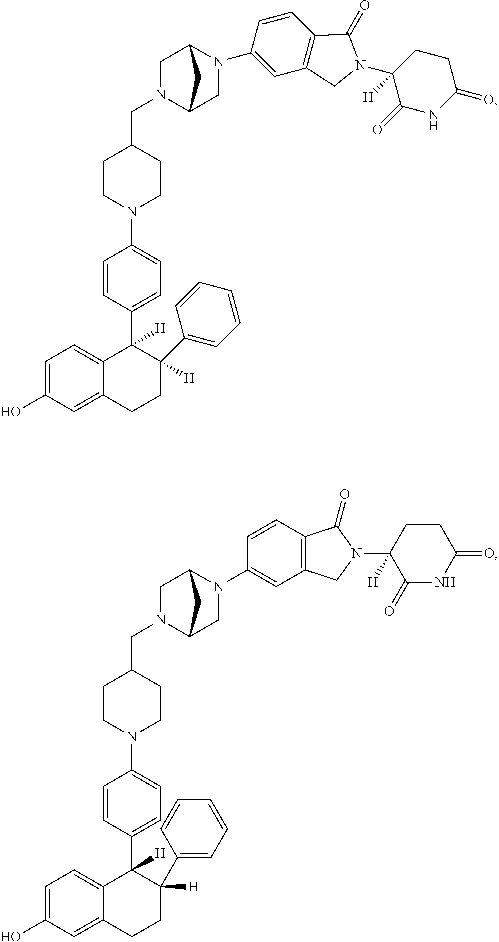 [SMILES: O=C1CC[C@H](N2Cc3cc(N4C[C@H]5C[C@@H]4CN5CC4CCN(c5ccc([C@@H]6c7ccc(O)cc7CC[C@@H]6c6ccccc6)cc5)CC4)ccc3C2=O)C(=O)N1 ; O=C1CC[C@H](N2Cc3cc(N4C[C@H]5C[C@@H]4CN5CC4CCN(c5ccc([C@H]6c7ccc(O)cc7CC[C@H]6c6ccccc6)cc5)CC4)ccc3C2=O)C(=O)N1] 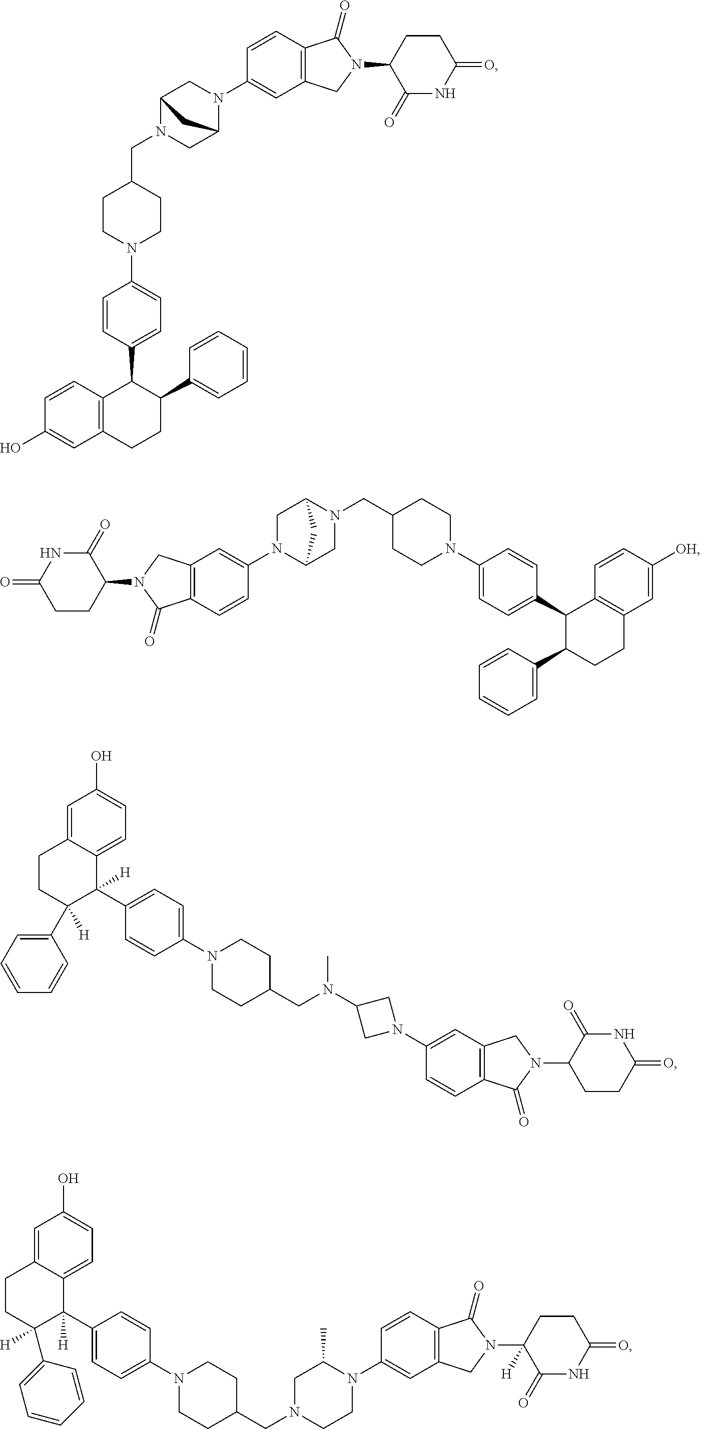 [SMILES: O=C1CC[C@H](N2Cc3cc(N4C[C@@H]5C[C@H]4CN5CC4CCN(c5ccc([C@@H]6c7ccc(O)cc7CC[C@@H]6c6ccccc6)cc5)CC4)ccc3C2=O)C(=O)N1 ; O=C1CC[C@H](N2Cc3cc(N4C[C@@H]5C[C@H]4CN5CC4CCN(c5ccc([C@H]6c7ccc(O)cc7CC[C@H]6c6ccccc6)cc5)CC4)ccc3C2=O)C(=O)N1 ; CN(CC1CCN(c2ccc([C@@H]3c4ccc(O)cc4CC[C@@H]3c3ccccc3)cc2)CC1)C1CN(c2ccc3c(c2)CN(C2CCC(=O)NC2=O)C3=O)C1 ; C[C@H]1CN(CC2CCN(c3ccc([C@@H]4c5ccc(O)cc5CC[C@@H]4c4ccccc4)cc3)CC2)CCN1c1ccc2c(c1)CN([C@H]1CCC(=O)NC1=O)C2=O] 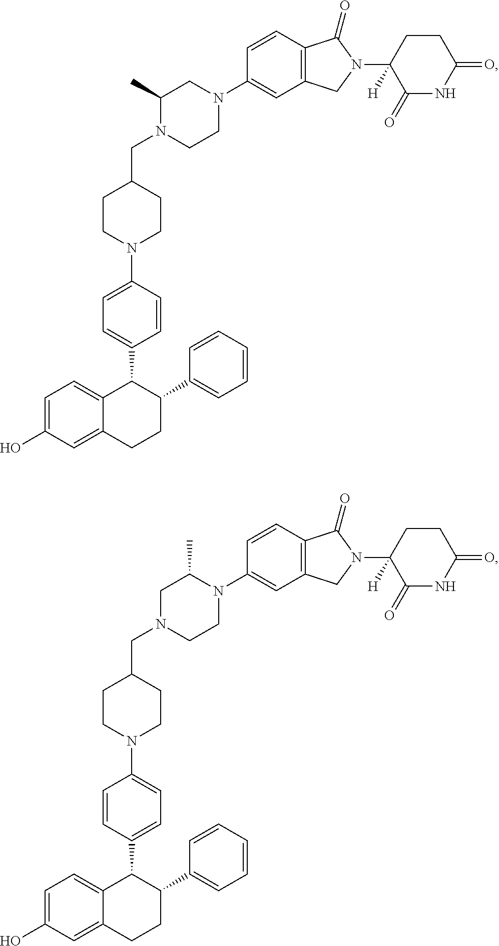 [SMILES: C[C@H]1CN(c2ccc3c(c2)CN([C@H]2CCC(=O)NC2=O)C3=O)CCN1CC1CCN(c2ccc([C@H]3c4ccc(O)cc4CC[C@H]3c3ccccc3)cc2)CC1 ; C[C@H]1CN(CC2CCN(c3ccc([C@H]4c5ccc(O)cc5CC[C@H]4c4ccccc4)cc3)CC2)CCN1c1ccc2c(c1)CN([C@H]1CCC(=O)NC1=O)C2=O] 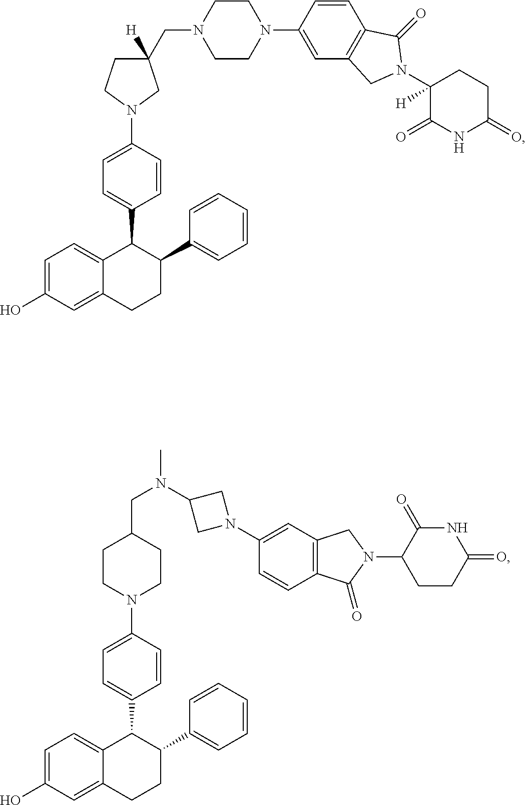 [SMILES: O=C1CC[C@H](N2Cc3cc(N4CCN(C[C@H]5CCN(c6ccc([C@@H]7c8ccc(O)cc8CC[C@@H]7c7ccccc7)cc6)C5)CC4)ccc3C2=O)C(=O)N1 ; CN(CC1CCN(c2ccc([C@H]3c4ccc(O)cc4CC[C@H]3c3ccccc3)cc2)CC1)C1CN(c2ccc3c(c2)CN(C2CCC(=O)NC2=O)C3=O)C1] 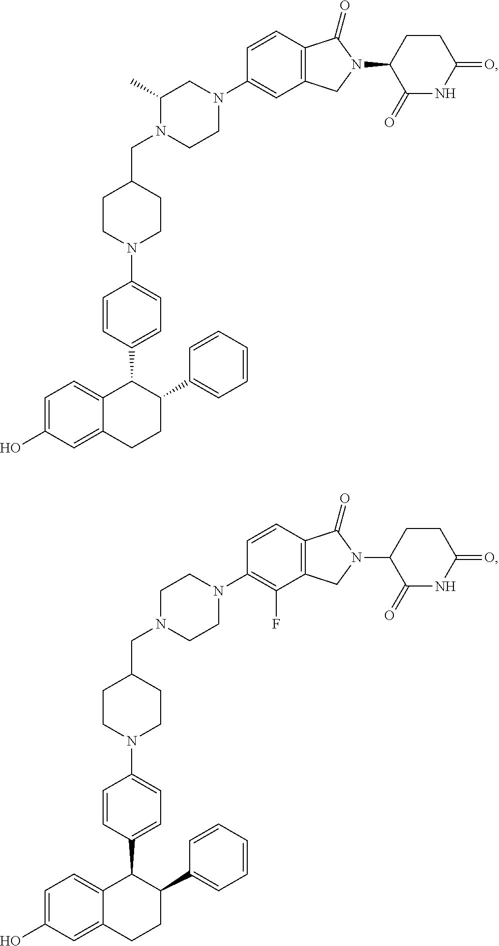 [SMILES: C[C@@H]1CN(c2ccc3c(c2)CN([C@H]2CCC(=O)NC2=O)C3=O)CCN1CC1CCN(c2ccc([C@H]3c4ccc(O)cc4CC[C@H]3c3ccccc3)cc2)CC1 ; O=C1CCC(N2Cc3c(ccc(N4CCN(CC5CCN(c6ccc([C@@H]7c8ccc(O)cc8CC[C@@H]7c7ccccc7)cc6)CC5)CC4)c3F)C2=O)C(=O)N1] 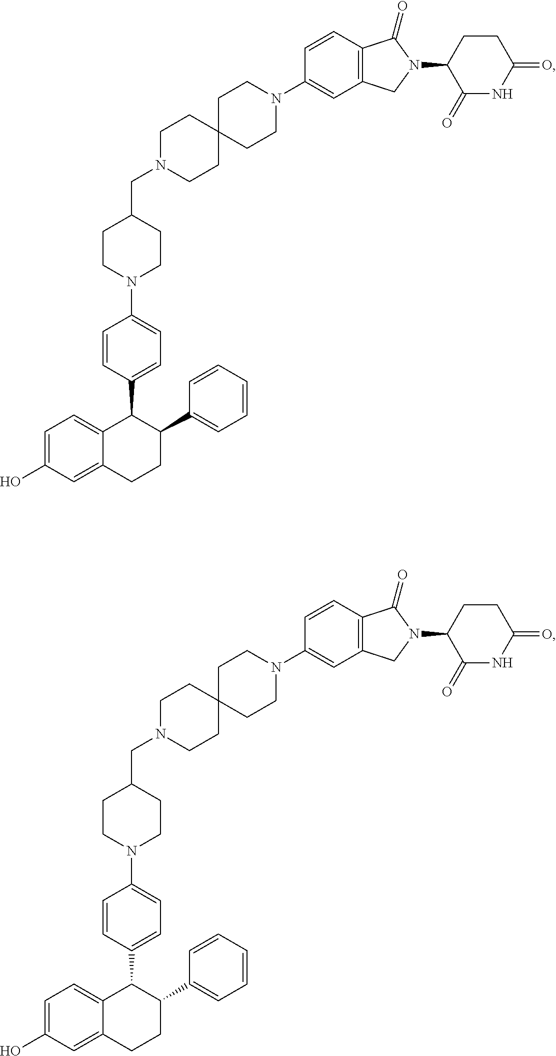 [SMILES: O=C1CC[C@H](N2Cc3cc(N4CCC5(CCN(CC6CCN(c7ccc([C@@H]8c9ccc(O)cc9CC[C@@H]8c8ccccc8)cc7)CC6)CC5)CC4)ccc3C2=O)C(=O)N1 ; O=C1CC[C@H](N2Cc3cc(N4CCC5(CCN(CC6CCN(c7ccc([C@H]8c9ccc(O)cc9CC[C@H]8c8ccccc8)cc7)CC6)CC5)CC4)ccc3C2=O)C(=O)N1] 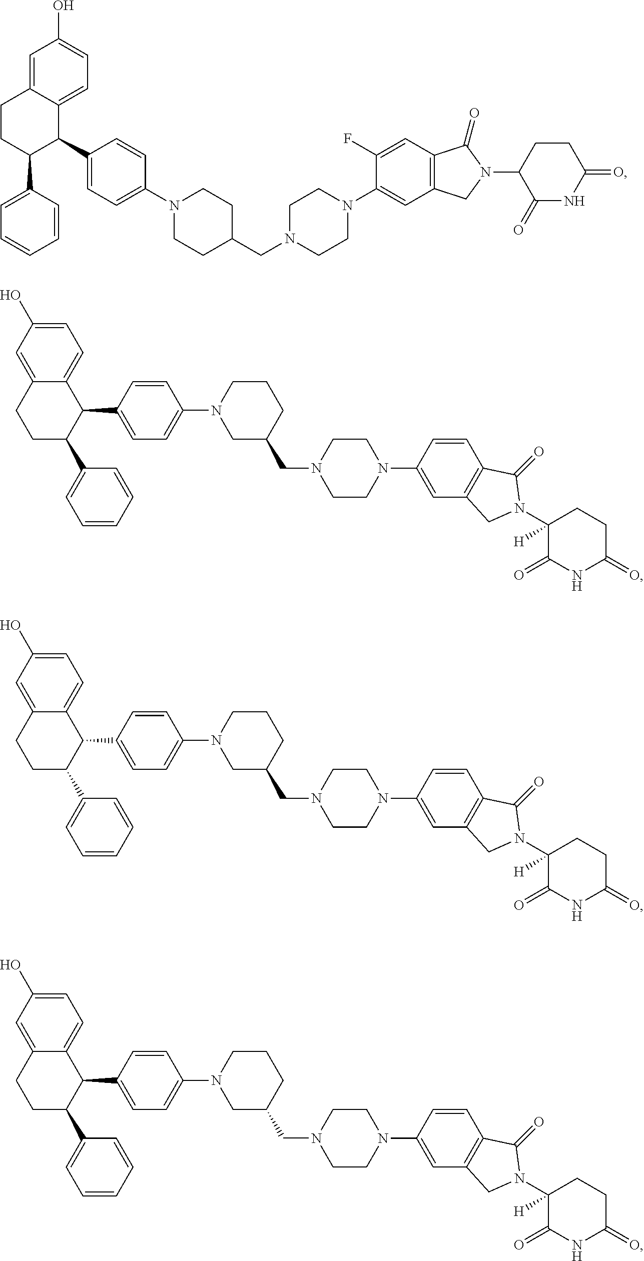 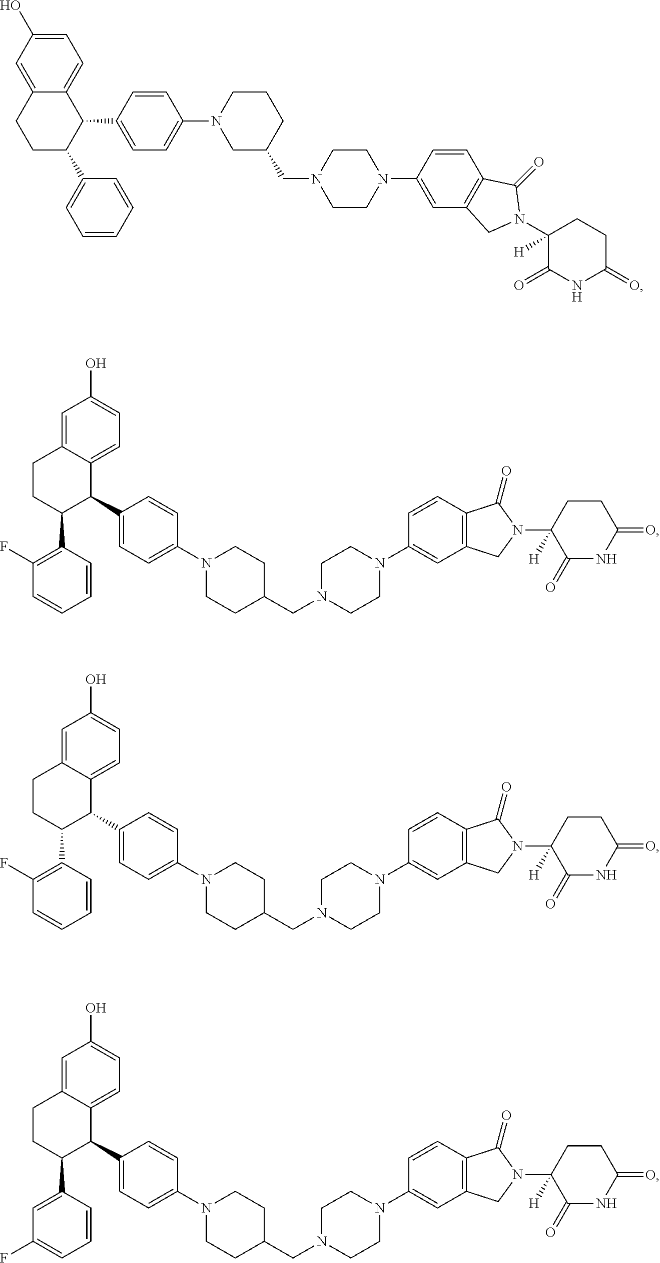 [SMILES: O=C1CC[C@H](N2Cc3cc(N4CCN(C[C@H]5CCCN(c6ccc([C@H]7c8ccc(O)cc8CC[C@H]7c7ccccc7)cc6)C5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccccc7F)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccccc7F)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc(F)c7)cc6)CC5)CC4)ccc3C2=O)C(=O)N1] 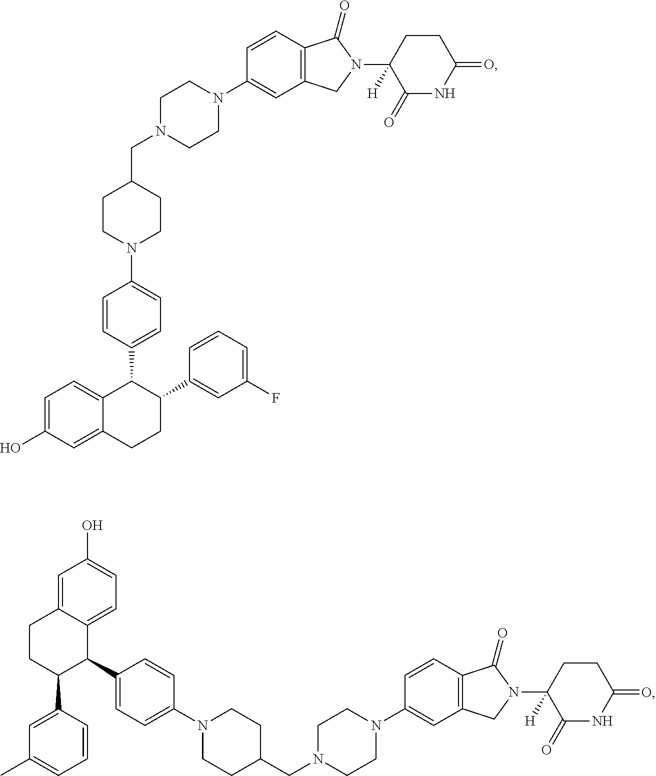 [SMILES: O=C1CC[C@H](N2Cc3cc(N4CCN(CC5CCN(c6ccc([C@H]7c8ccc(O)cc8CC[C@H]7c7cccc(F)c7)cc6)CC5)CC4)ccc3C2=O)C(=O)N1 ; Cc1cccc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)c1] 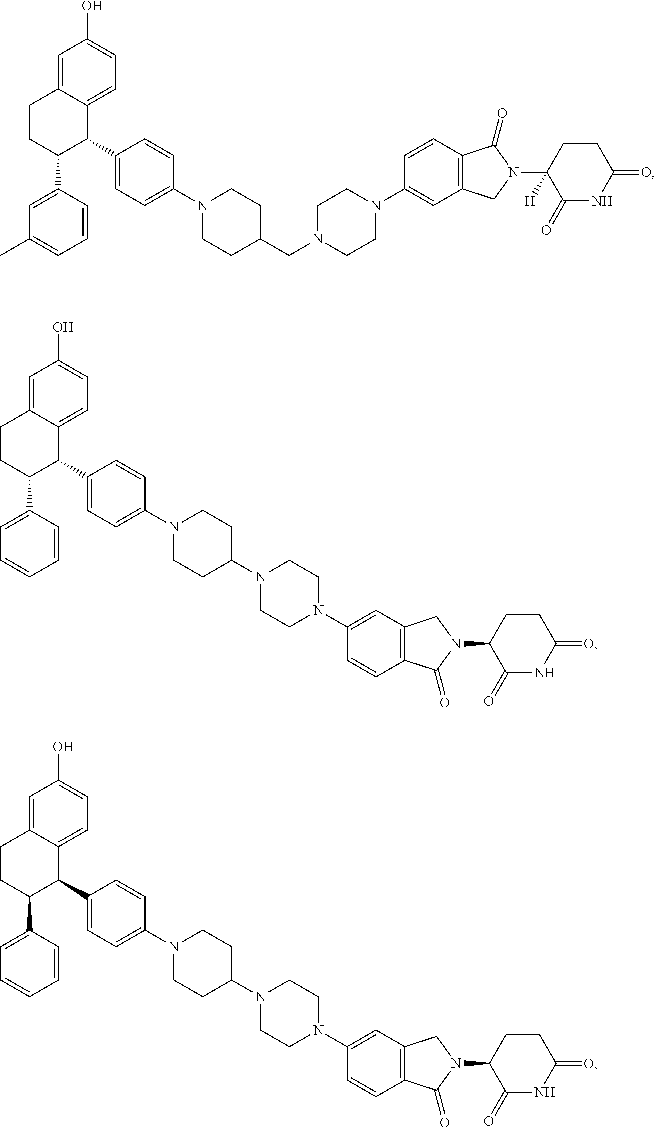 [SMILES: Cc1cccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)c1 ; O=C1CC[C@H](N2Cc3cc(N4CCN(C5CCN(c6ccc([C@H]7c8ccc(O)cc8CC[C@H]7c7ccccc7)cc6)CC5)CC4)ccc3C2=O)C(=O)N1 ; O=C1CC[C@H](N2Cc3cc(N4CCN(C5CCN(c6ccc([C@@H]7c8ccc(O)cc8CC[C@@H]7c7ccccc7)cc6)CC5)CC4)ccc3C2=O)C(=O)N1] 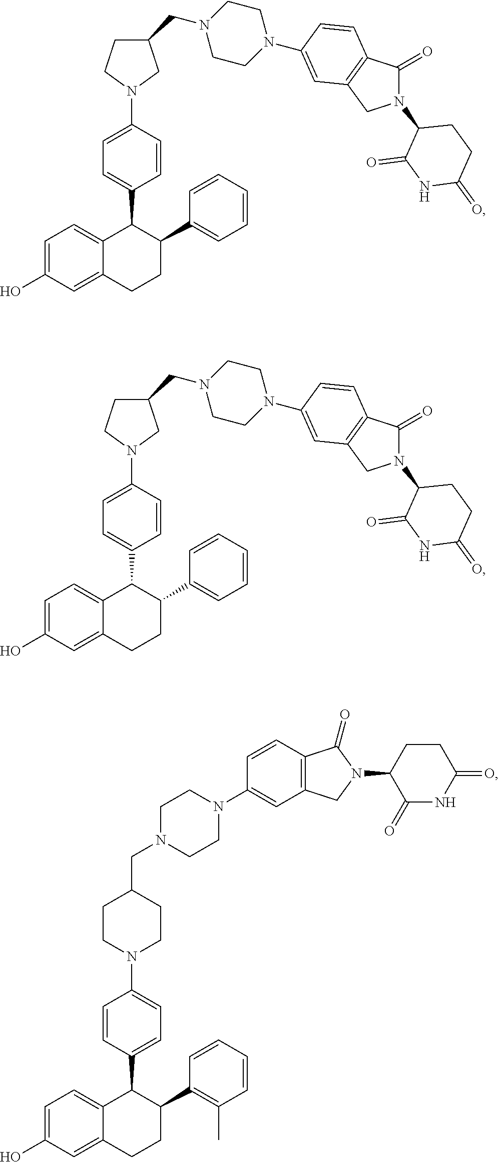 [SMILES: O=C1CC[C@H](N2Cc3cc(N4CCN(C[C@@H]5CCN(c6ccc([C@@H]7c8ccc(O)cc8CC[C@@H]7c7ccccc7)cc6)C5)CC4)ccc3C2=O)C(=O)N1 ; O=C1CC[C@H](N2Cc3cc(N4CCN(C[C@@H]5CCN(c6ccc([C@H]7c8ccc(O)cc8CC[C@H]7c7ccccc7)cc6)C5)CC4)ccc3C2=O)C(=O)N1 ; Cc1ccccc1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1] 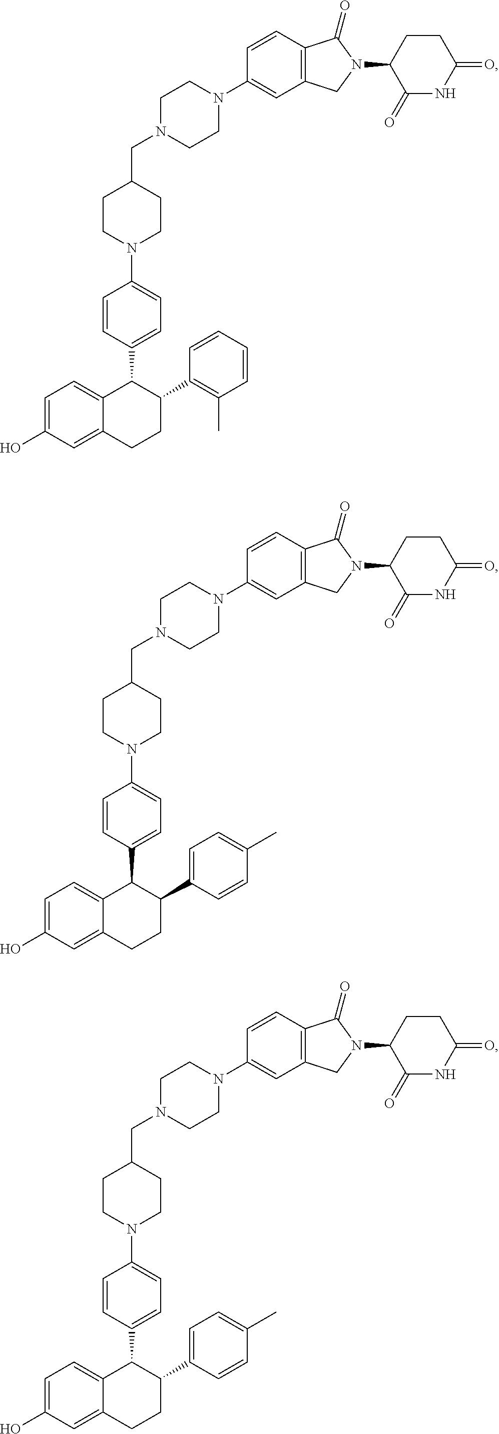 [SMILES: Cc1ccccc1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; Cc1ccc([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)cc1 ; Cc1ccc([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1] 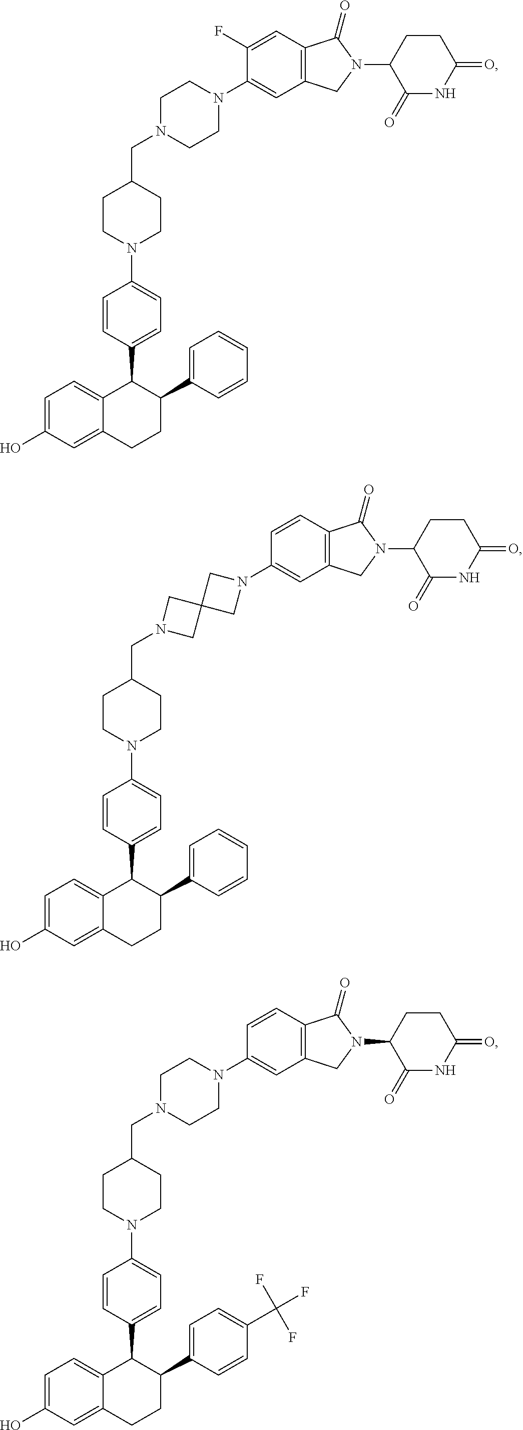 [SMILES: O=C1CCC(N2Cc3cc(N4CCN(CC5CCN(c6ccc([C@@H]7c8ccc(O)cc8CC[C@@H]7c7ccccc7)cc6)CC5)CC4)c(F)cc3C2=O)C(=O)N1 ; O=C1CCC(N2Cc3cc(N4CC5(CN(CC6CCN(c7ccc([C@@H]8c9ccc(O)cc9CC[C@@H]8c8ccccc8)cc7)CC6)C5)C4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccc(C(F)(F)F)cc7)cc6)CC5)CC4)ccc3C2=O)C(=O)N1] 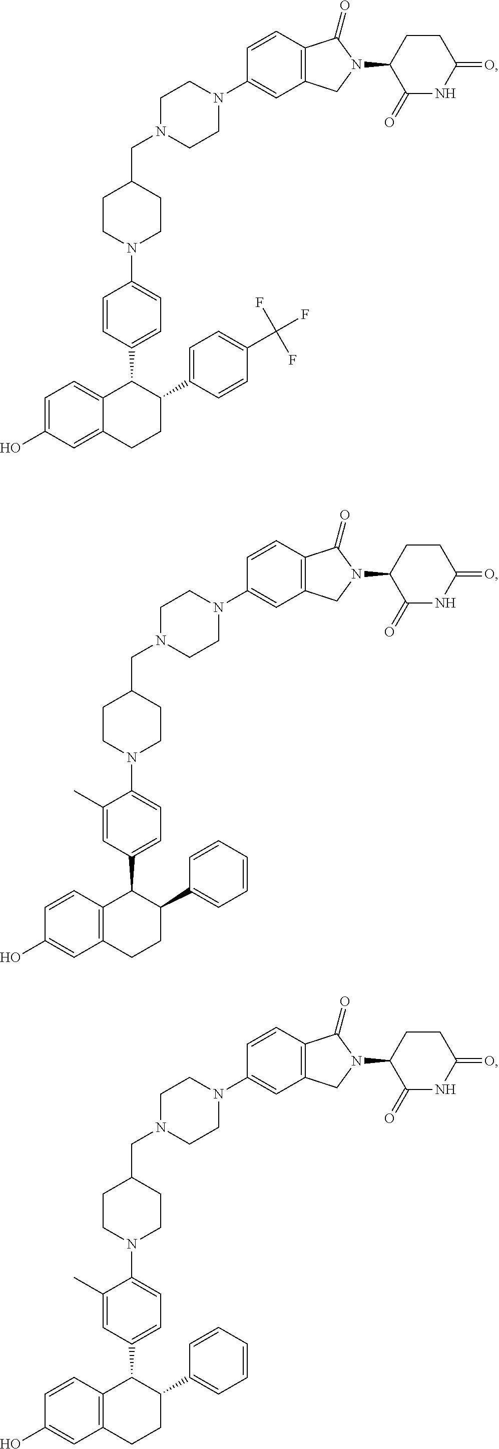 [SMILES: O=C1CC[C@H](N2Cc3cc(N4CCN(CC5CCN(c6ccc([C@H]7c8ccc(O)cc8CC[C@H]7c7ccc(C(F)(F)F)cc7)cc6)CC5)CC4)ccc3C2=O)C(=O)N1 ; Cc1cc([C@@H]2c3ccc(O)cc3CC[C@@H]2c2ccccc2)ccc1N1CCC(CN2CCN(c3ccc4c(c3)CN([C@H]3CCC(=O)NC3=O)C4=O)CC2)CC1 ; Cc1cc([C@H]2c3ccc(O)cc3CC[C@H]2c2ccccc2)ccc1N1CCC(CN2CCN(c3ccc4c(c3)CN([C@H]3CCC(=O)NC3=O)C4=O)CC2)CC1] 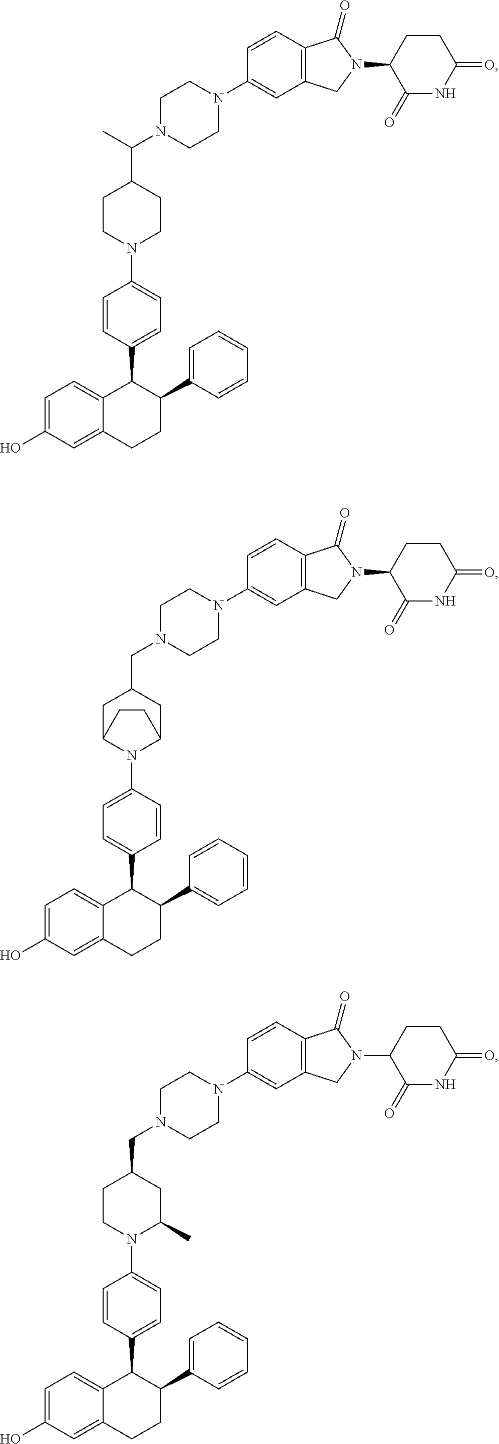 [SMILES: CC(C1CCN(c2ccc([C@@H]3c4ccc(O)cc4CC[C@@H]3c3ccccc3)cc2)CC1)N1CCN(c2ccc3c(c2)CN([C@H]2CCC(=O)NC2=O)C3=O)CC1 ; O=C1CC[C@H](N2Cc3cc(N4CCN(CC5CC6CCC(C5)N6c5ccc([C@@H]6c7ccc(O)cc7CC[C@@H]6c6ccccc6)cc5)CC4)ccc3C2=O)C(=O)N1 ; C[C@@H]1C[C@H](CN2CCN(c3ccc4c(c3)CN(C3CCC(=O)NC3=O)C4=O)CC2)CCN1c1ccc([C@@H]2c3ccc(O)cc3CC[C@@H]2c2ccccc2)cc1] 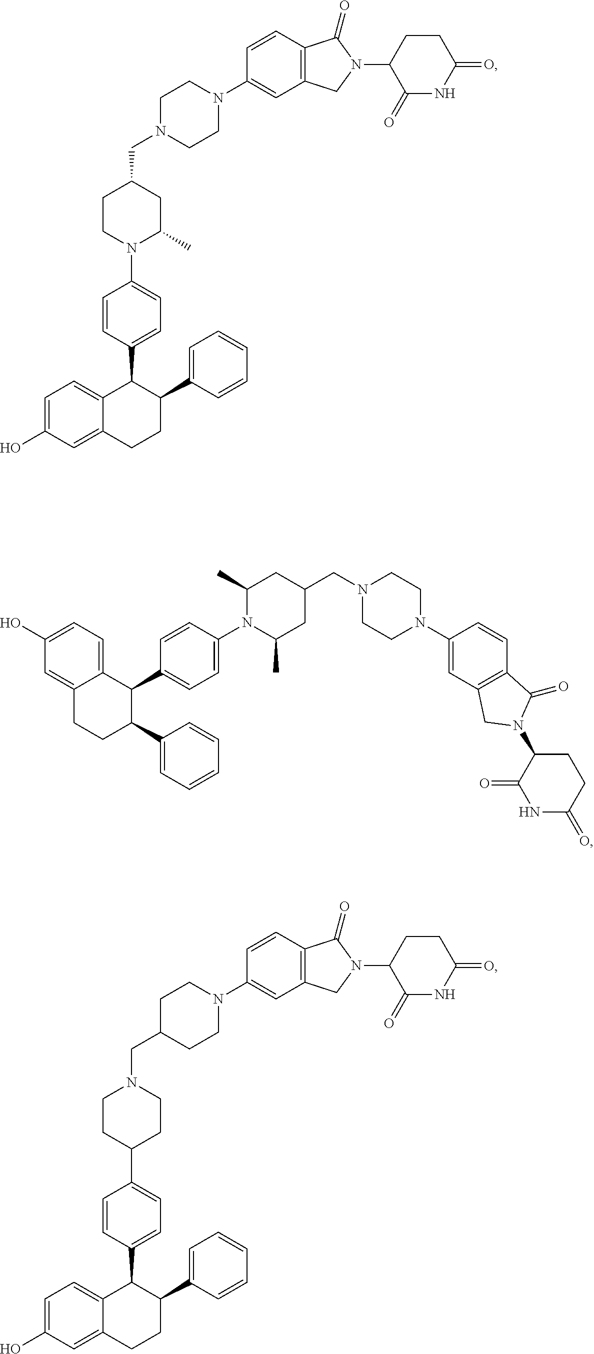 [SMILES: C[C@H]1C[C@@H](CN2CCN(c3ccc4c(c3)CN(C3CCC(=O)NC3=O)C4=O)CC2)CCN1c1ccc([C@@H]2c3ccc(O)cc3CC[C@@H]2c2ccccc2)cc1 ; C[C@@H]1CC(CN2CCN(c3ccc4c(c3)CN([C@H]3CCC(=O)NC3=O)C4=O)CC2)C[C@H](C)N1c1ccc([C@@H]2c3ccc(O)cc3CC[C@@H]2c2ccccc2)cc1 ; O=C1CCC(N2Cc3cc(N4CCC(CN5CCC(c6ccc([C@@H]7c8ccc(O)cc8CC[C@@H]7c7ccccc7)cc6)CC5)CC4)ccc3C2=O)C(=O)N1] 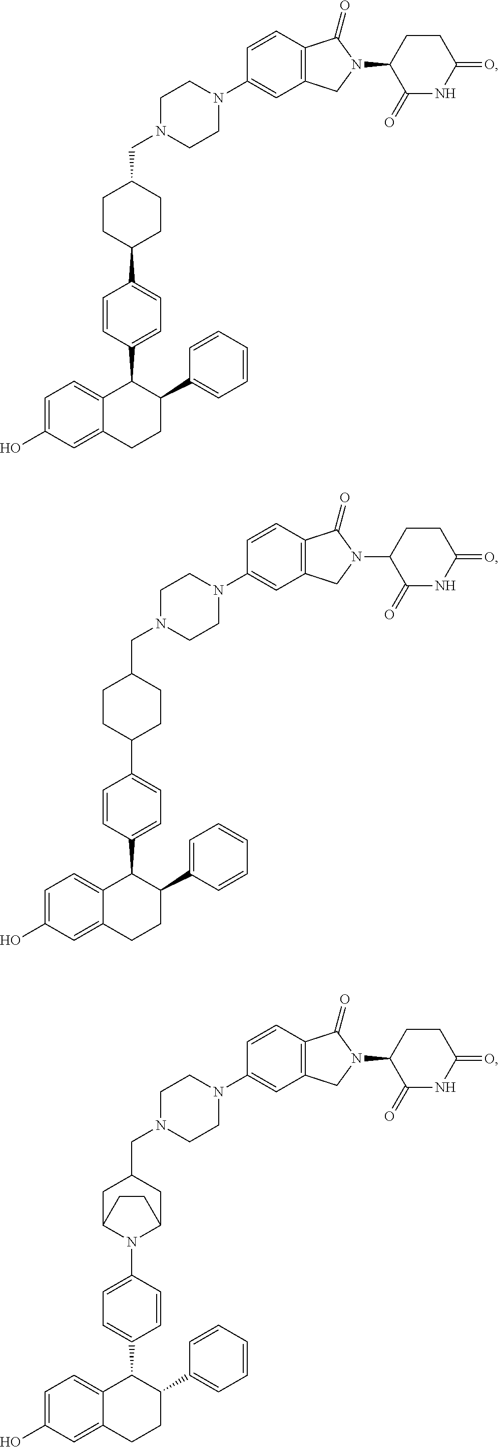 [SMILES: O=C1CC[C@H](N2Cc3cc(N4CCN(C[C@H]5CC[C@H](c6ccc([C@@H]7c8ccc(O)cc8CC[C@@H]7c7ccccc7)cc6)CC5)CC4)ccc3C2=O)C(=O)N1 ; O=C1CCC(N2Cc3cc(N4CCN(CC5CCC(c6ccc([C@@H]7c8ccc(O)cc8CC[C@@H]7c7ccccc7)cc6)CC5)CC4)ccc3C2=O)C(=O)N1 ; O=C1CC[C@H](N2Cc3cc(N4CCN(CC5CC6CCC(C5)N6c5ccc([C@H]6c7ccc(O)cc7CC[C@H]6c6ccccc6)cc5)CC4)ccc3C2=O)C(=O)N1] 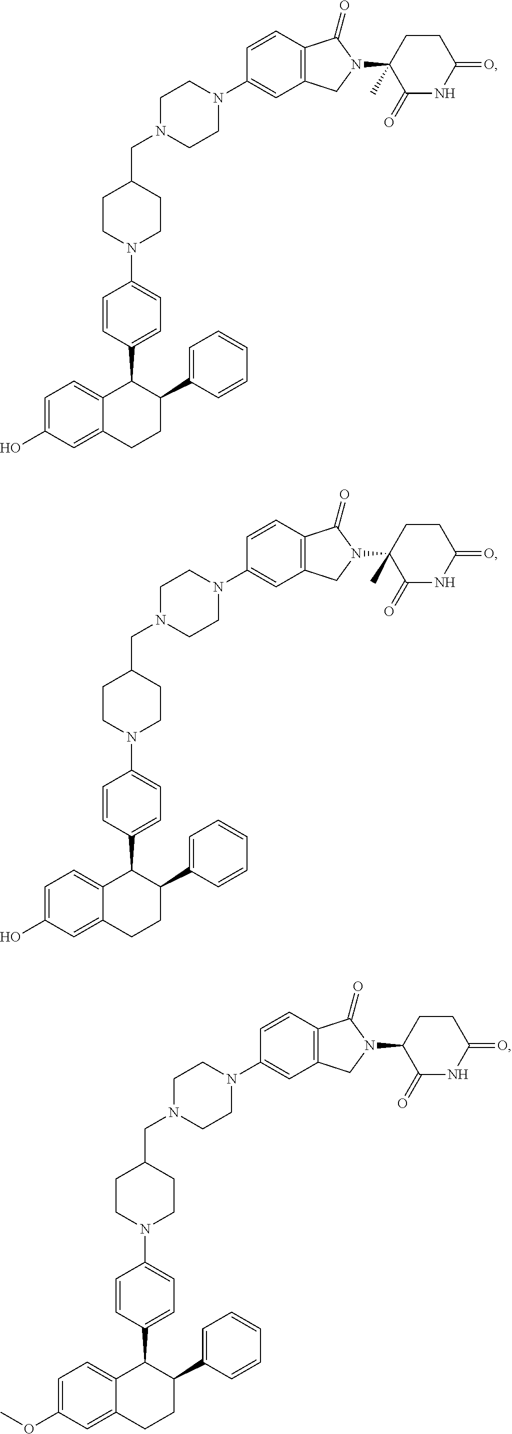 [SMILES: C[C@]1(N2Cc3cc(N4CCN(CC5CCN(c6ccc([C@@H]7c8ccc(O)cc8CC[C@@H]7c7ccccc7)cc6)CC5)CC4)ccc3C2=O)CCC(=O)NC1=O ; C[C@@]1(N2Cc3cc(N4CCN(CC5CCN(c6ccc([C@@H]7c8ccc(O)cc8CC[C@@H]7c7ccccc7)cc6)CC5)CC4)ccc3C2=O)CCC(=O)NC1=O ; COc1ccc2c(c1)CC[C@H](c1ccccc1)[C@@H]2c1ccc(N2CCC(CN3CCN(c4ccc5c(c4)CN([C@H]4CCC(=O)NC4=O)C5=O)CC3)CC2)cc1] 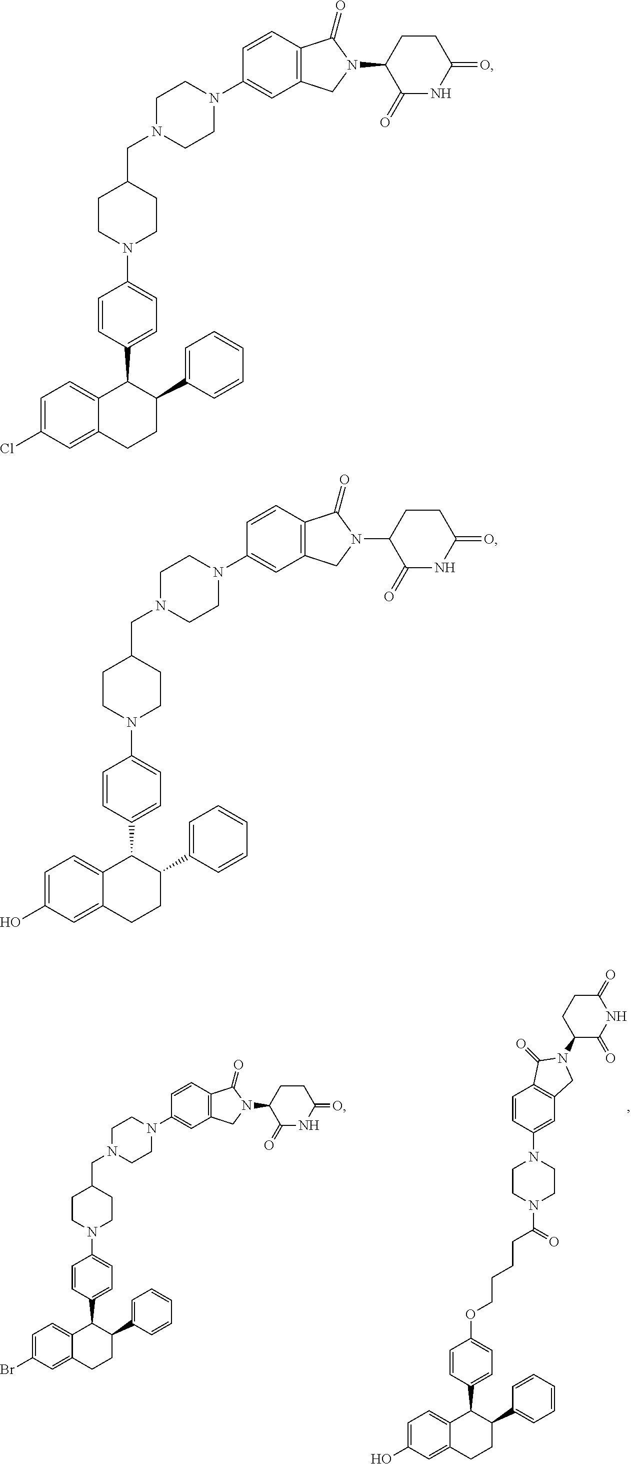 [SMILES: O=C1CC[C@H](N2Cc3cc(N4CCN(CC5CCN(c6ccc([C@@H]7c8ccc(Cl)cc8CC[C@@H]7c7ccccc7)cc6)CC5)CC4)ccc3C2=O)C(=O)N1 ; O=C1CCC(N2Cc3cc(N4CCN(CC5CCN(c6ccc([C@H]7c8ccc(O)cc8CC[C@H]7c7ccccc7)cc6)CC5)CC4)ccc3C2=O)C(=O)N1 ; O=C1CC[C@H](N2Cc3cc(N4CCN(CC5CCN(c6ccc([C@@H]7c8ccc(Br)cc8CC[C@@H]7c7ccccc7)cc6)CC5)CC4)ccc3C2=O)C(=O)N1 ; O=C1CC[C@H](N2Cc3cc(N4CCN(C(=O)CCCCOc5ccc([C@@H]6c7ccc(O)cc7CC[C@@H]6c6ccccc6)cc5)CC4)ccc3C2=O)C(=O)N1] 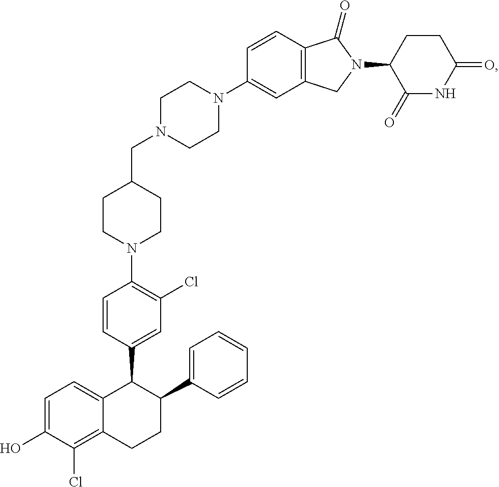 [SMILES: O=C1CC[C@H](N2Cc3cc(N4CCN(CC5CCN(c6ccc([C@@H]7c8ccc(O)c(Cl)c8CC[C@@H]7c7ccccc7)cc6Cl)CC5)CC4)ccc3C2=O)C(=O)N1] 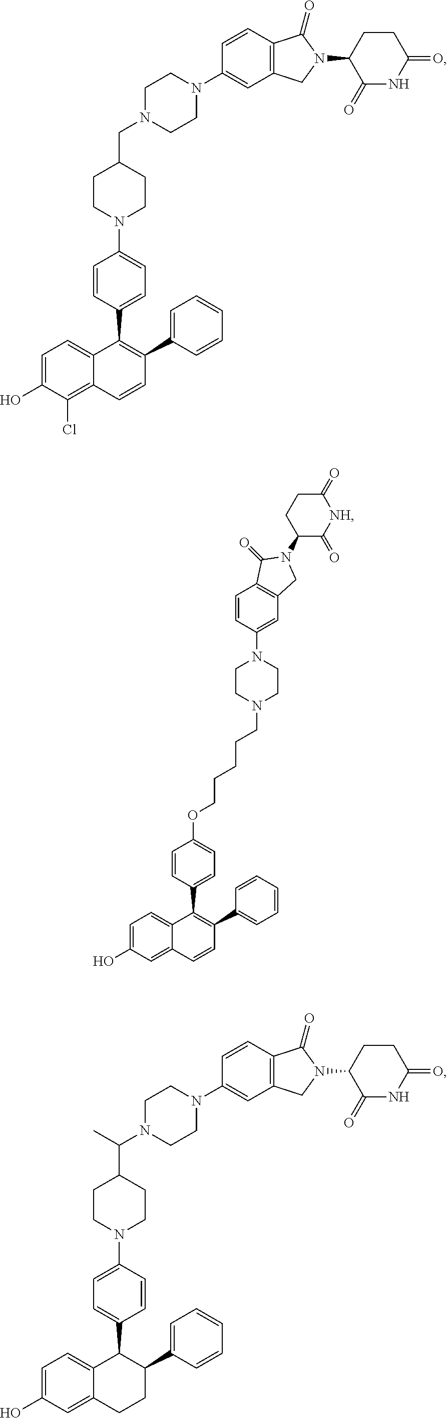 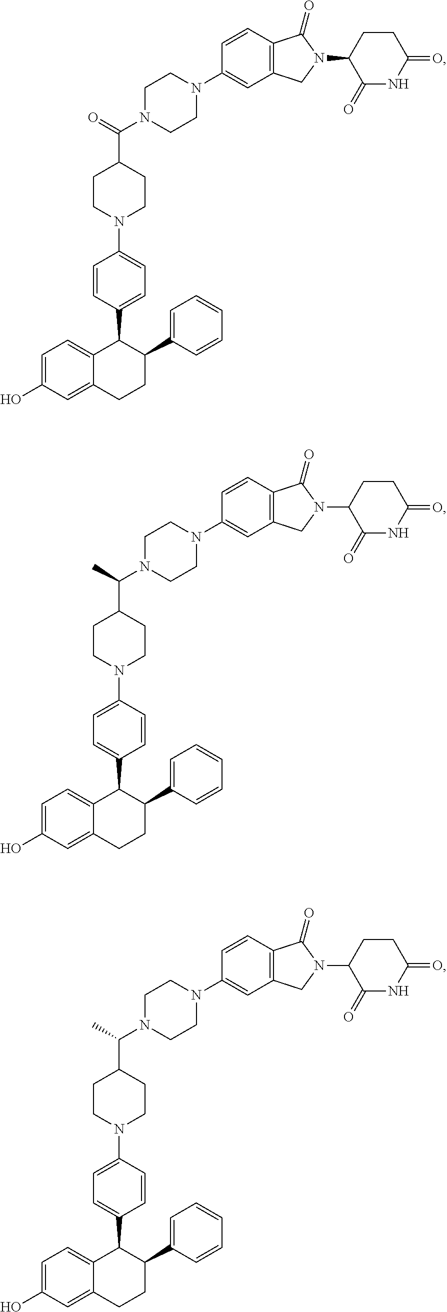 [SMILES: O=C1CC[C@H](N2Cc3cc(N4CCN(C(=O)C5CCN(c6ccc([C@@H]7c8ccc(O)cc8CC[C@@H]7c7ccccc7)cc6)CC5)CC4)ccc3C2=O)C(=O)N1 ; C[C@H](C1CCN(c2ccc([C@@H]3c4ccc(O)cc4CC[C@@H]3c3ccccc3)cc2)CC1)N1CCN(c2ccc3c(c2)CN(C2CCC(=O)NC2=O)C3=O)CC1 ; C[C@@H](C1CCN(c2ccc([C@@H]3c4ccc(O)cc4CC[C@@H]3c3ccccc3)cc2)CC1)N1CCN(c2ccc3c(c2)CN(C2CCC(=O)NC2=O)C3=O)CC1] 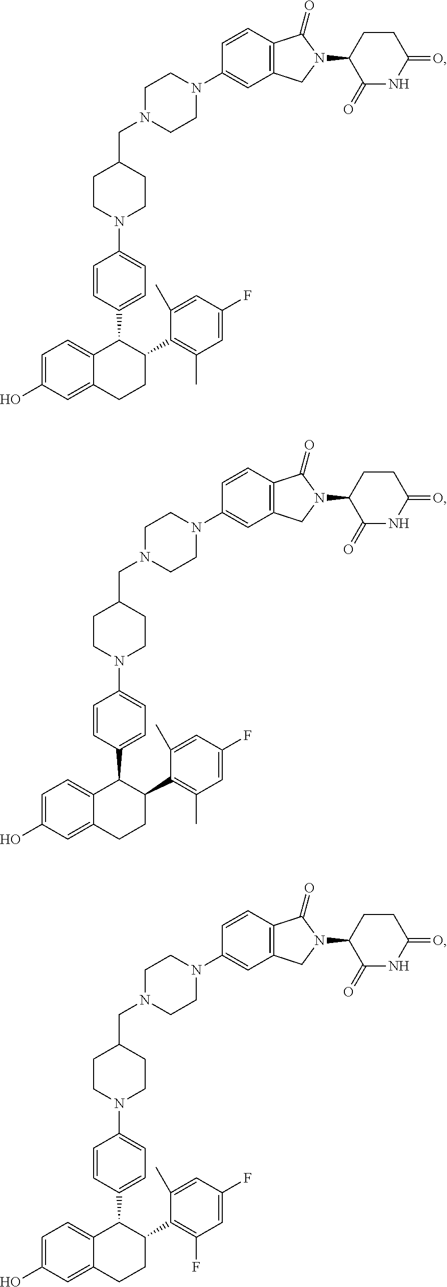 [SMILES: Cc1cc(F)cc(C)c1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1 ; Cc1cc(F)cc(C)c1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; Cc1cc(F)cc(F)c1[C@@H]1CCc2cc(O)ccc2[C@@H]1c1ccc(N2CCC(CN3CCN(c4ccc5c(c4)CN([C@H]4CCC(=O)NC4=O)C5=O)CC3)CC2)cc1] 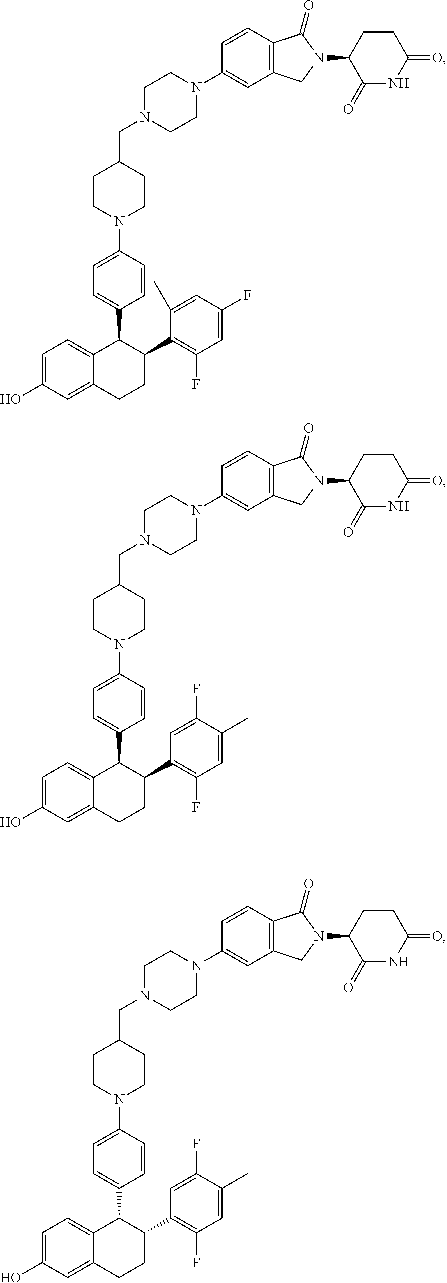 [SMILES: Cc1cc(F)cc(F)c1[C@H]1CCc2cc(O)ccc2[C@H]1c1ccc(N2CCC(CN3CCN(c4ccc5c(c4)CN([C@H]4CCC(=O)NC4=O)C5=O)CC3)CC2)cc1 ; Cc1cc(F)c([C@H]2CCc3cc(O)ccc3[C@H]2c2ccc(N3CCC(CN4CCN(c5ccc6c(c5)CN([C@H]5CCC(=O)NC5=O)C6=O)CC4)CC3)cc2)cc1F ; Cc1cc(F)c([C@@H]2CCc3cc(O)ccc3[C@@H]2c2ccc(N3CCC(CN4CCN(c5ccc6c(c5)CN([C@H]5CCC(=O)NC5=O)C6=O)CC4)CC3)cc2)cc1F] 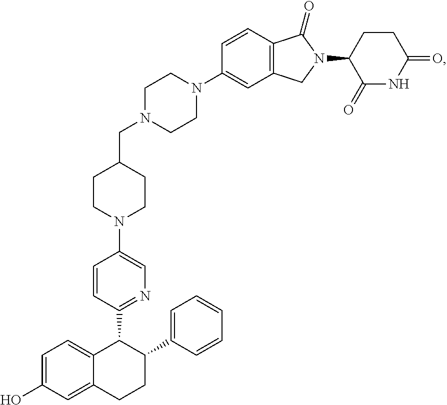 [SMILES: O=C1CC[C@H](N2Cc3cc(N4CCN(CC5CCN(c6ccc([C@H]7c8ccc(O)cc8CC[C@H]7c7ccccc7)nc6)CC5)CC4)ccc3C2=O)C(=O)N1] 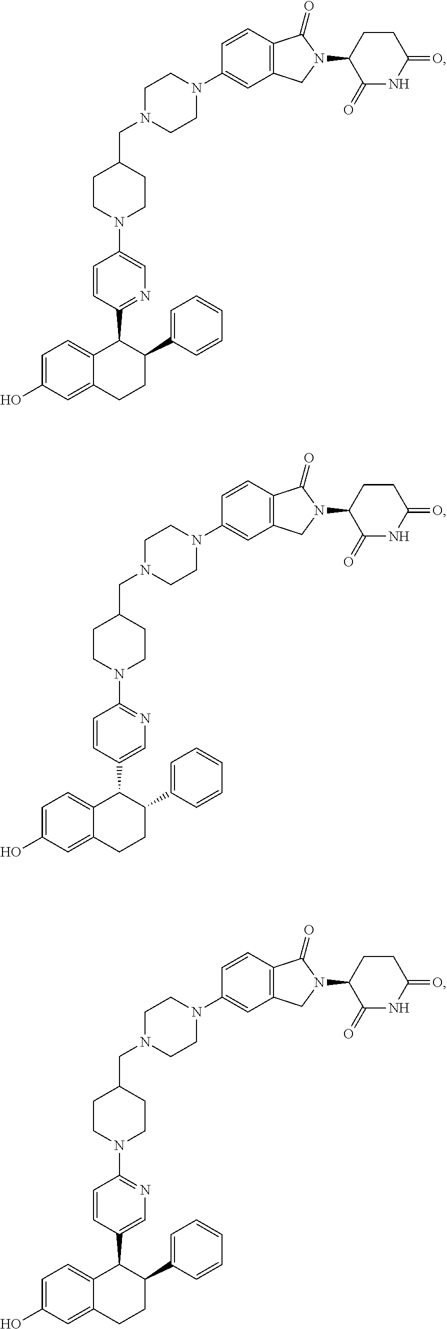 [SMILES: O=C1CC[C@H](N2Cc3cc(N4CCN(CC5CCN(c6ccc([C@@H]7c8ccc(O)cc8CC[C@@H]7c7ccccc7)nc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccccc7)cn6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccccc7)cn6)CC5)CC4)ccc3C2=O)C(=O)N1] 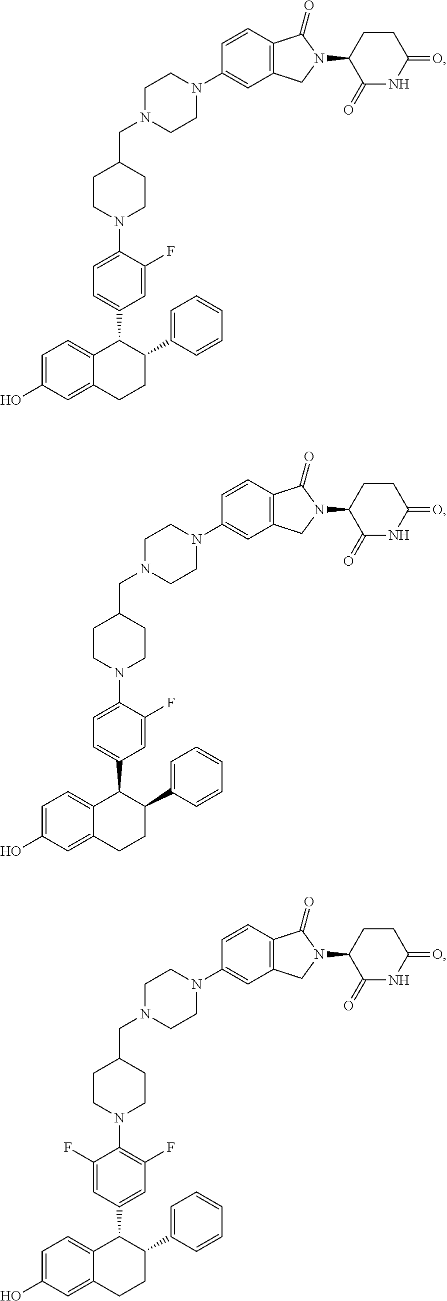 [SMILES: O=C1CC[C@H](N2Cc3cc(N4CCN(CC5CCN(c6ccc([C@H]7c8ccc(O)cc8CC[C@H]7c7ccccc7)cc6F)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccccc7)cc6F)CC5)CC4)ccc3C2=O)C(=O)N1 ; O=C1CC[C@H](N2Cc3cc(N4CCN(CC5CCN(c6c(F)cc([C@H]7c8ccc(O)cc8CC[C@H]7c7ccccc7)cc6F)CC5)CC4)ccc3C2=O)C(=O)N1] 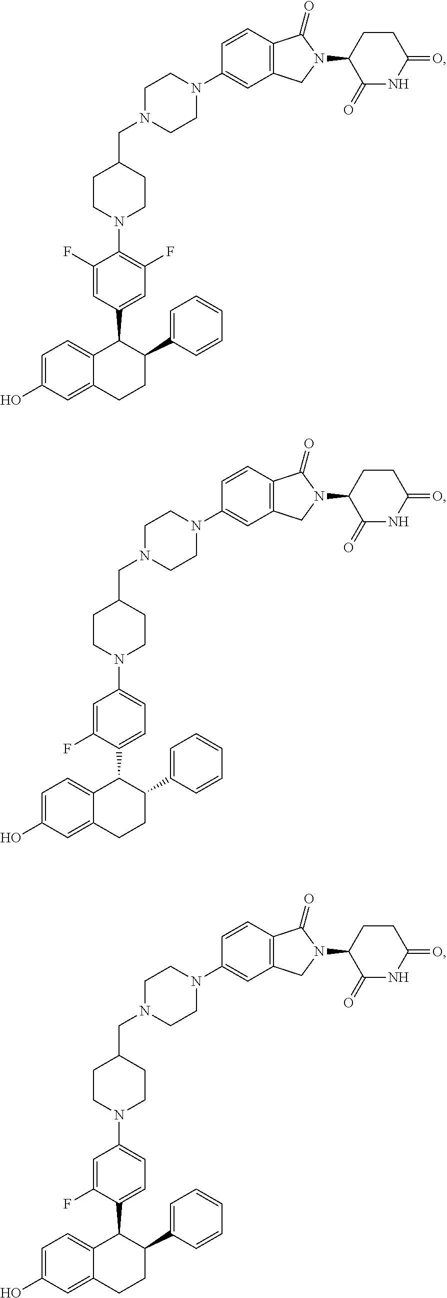 [SMILES: O=C1CC[C@H](N2Cc3cc(N4CCN(CC5CCN(c6c(F)cc([C@@H]7c8ccc(O)cc8CC[C@@H]7c7ccccc7)cc6F)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccccc7)c(F)c6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccccc7)c(F)c6)CC5)CC4)ccc3C2=O)C(=O)N1] 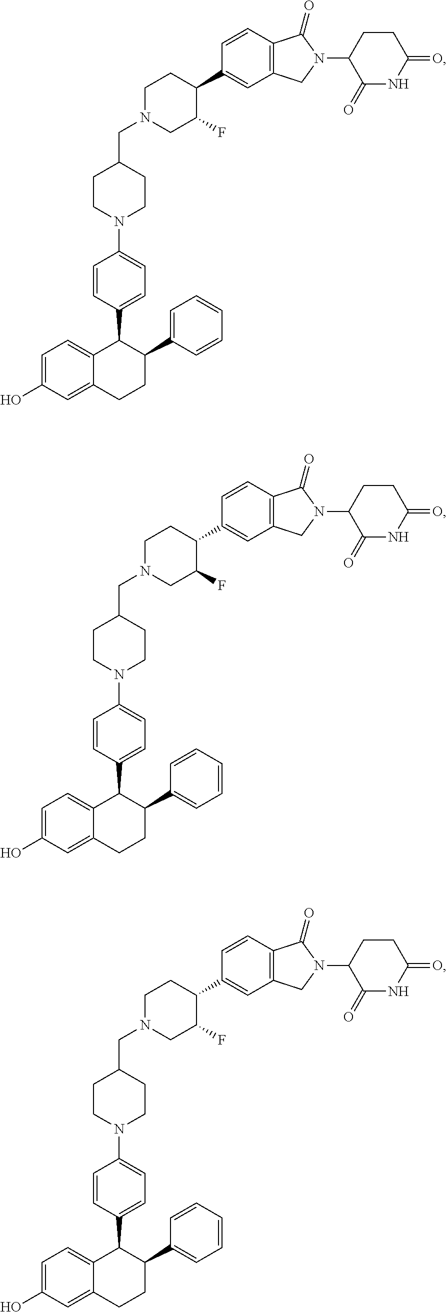 [SMILES: O=C1CCC(N2Cc3cc([C@@H]4CCN(CC5CCN(c6ccc([C@@H]7c8ccc(O)cc8CC[C@@H]7c7ccccc7)cc6)CC5)C[C@H]4F)ccc3C2=O)C(=O)N1 ; O=C1CCC(N2Cc3cc([C@H]4CCN(CC5CCN(c6ccc([C@@H]7c8ccc(O)cc8CC[C@@H]7c7ccccc7)cc6)CC5)C[C@@H]4F)ccc3C2=O)C(=O)N1 ; O=C1CCC(N2Cc3cc([C@H]4CCN(CC5CCN(c6ccc([C@@H]7c8ccc(O)cc8CC[C@@H]7c7ccccc7)cc6)CC5)C[C@H]4F)ccc3C2=O)C(=O)N1] 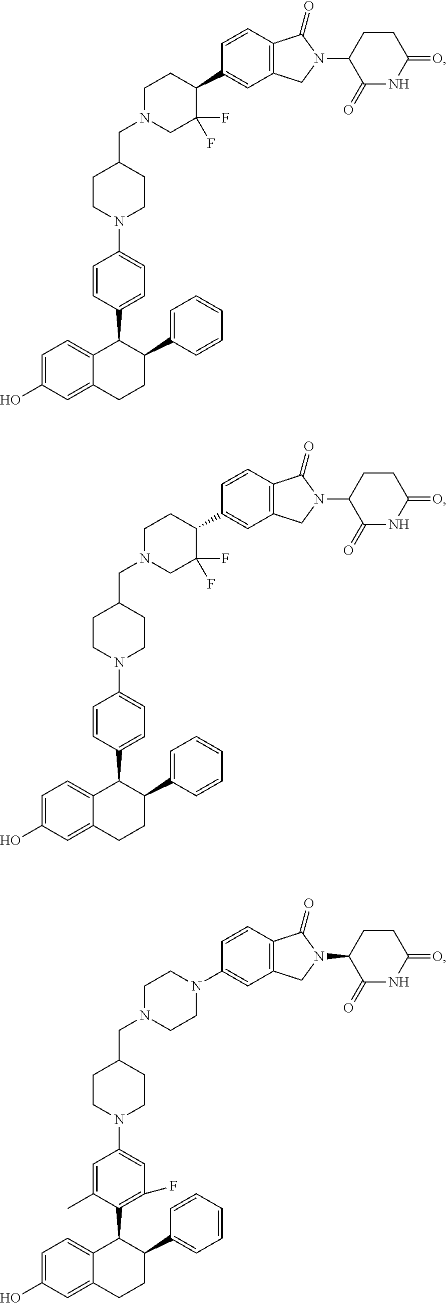 [SMILES: O=C1CCC(N2Cc3cc([C@@H]4CCN(CC5CCN(c6ccc([C@@H]7c8ccc(O)cc8CC[C@@H]7c7ccccc7)cc6)CC5)CC4(F)F)ccc3C2=O)C(=O)N1 ; O=C1CCC(N2Cc3cc([C@H]4CCN(CC5CCN(c6ccc([C@@H]7c8ccc(O)cc8CC[C@@H]7c7ccccc7)cc6)CC5)CC4(F)F)ccc3C2=O)C(=O)N1 ; Cc1cc(N2CCC(CN3CCN(c4ccc5c(c4)CN([C@H]4CCC(=O)NC4=O)C5=O)CC3)CC2)cc(F)c1[C@@H]1c2ccc(O)cc2CC[C@@H]1c1ccccc1] 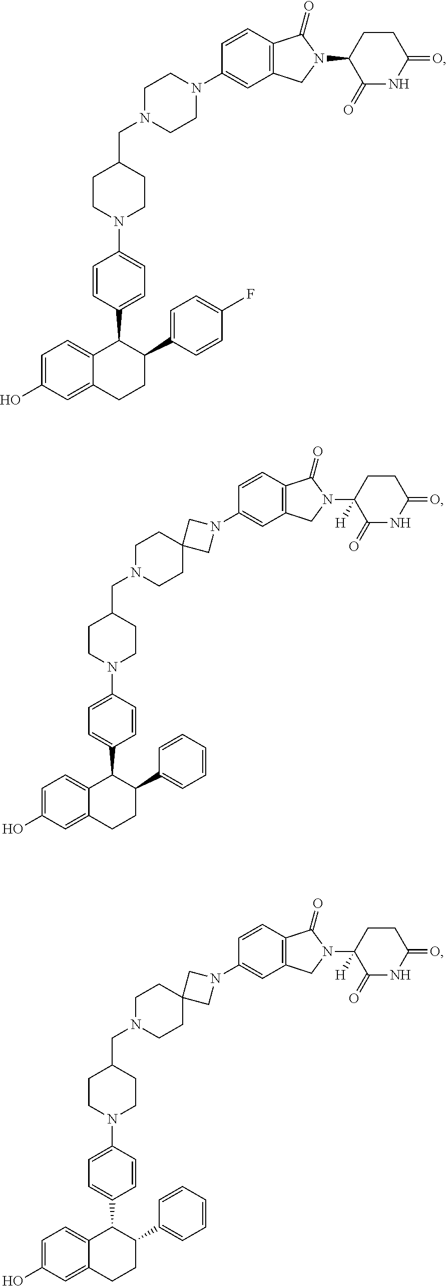 [SMILES: O=C1CC[C@H](N2Cc3cc(N4CCN(CC5CCN(c6ccc([C@@H]7c8ccc(O)cc8CC[C@@H]7c7ccc(F)cc7)cc6)CC5)CC4)ccc3C2=O)C(=O)N1 ; O=C1CC[C@H](N2Cc3cc(N4CC5(CCN(CC6CCN(c7ccc([C@@H]8c9ccc(O)cc9CC[C@@H]8c8ccccc8)cc7)CC6)CC5)C4)ccc3C2=O)C(=O)N1 ; O=C1CC[C@H](N2Cc3cc(N4CC5(CCN(CC6CCN(c7ccc([C@H]8c9ccc(O)cc9CC[C@H]8c8ccccc8)cc7)CC6)CC5)C4)ccc3C2=O)C(=O)N1] 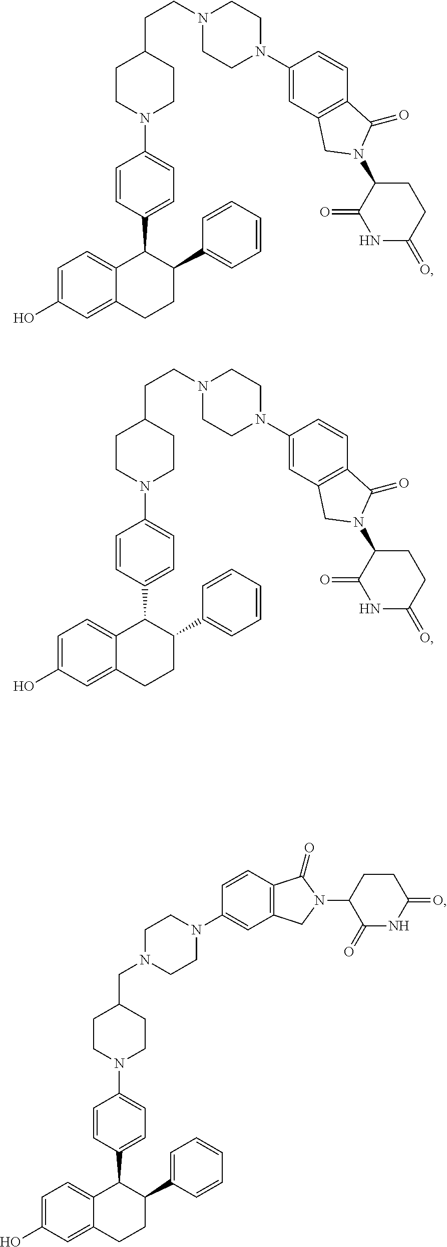 [SMILES: O=C1CC[C@H](N2Cc3cc(N4CCN(CCC5CCN(c6ccc([C@@H]7c8ccc(O)cc8CC[C@@H]7c7ccccc7)cc6)CC5)CC4)ccc3C2=O)C(=O)N1 ; O=C1CC[C@H](N2Cc3cc(N4CCN(CCC5CCN(c6ccc([C@H]7c8ccc(O)cc8CC[C@H]7c7ccccc7)cc6)CC5)CC4)ccc3C2=O)C(=O)N1 ; O=C1CCC(N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1] 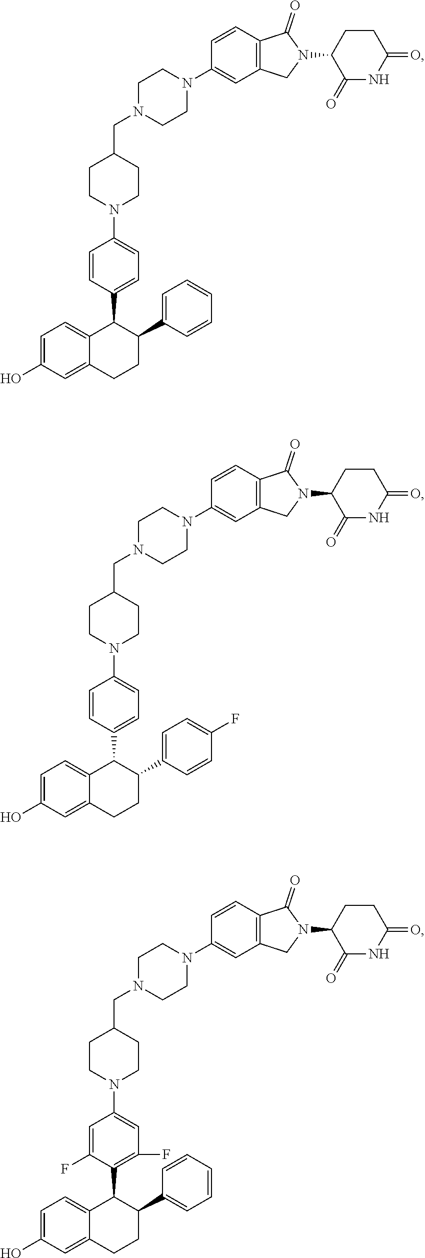 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc(F)cc7)cc6)CC5)CC4)ccc3C2=O)C(=O)N1 ; O=C1CC[C@H](N2Cc3cc(N4CCN(CC5CCN(c6cc(F)c([C@@H]7c8ccc(O)cc8CC[C@@H]7c7ccccc7)c(F)c6)CC5)CC4)ccc3C2=O)C(=O)N1] 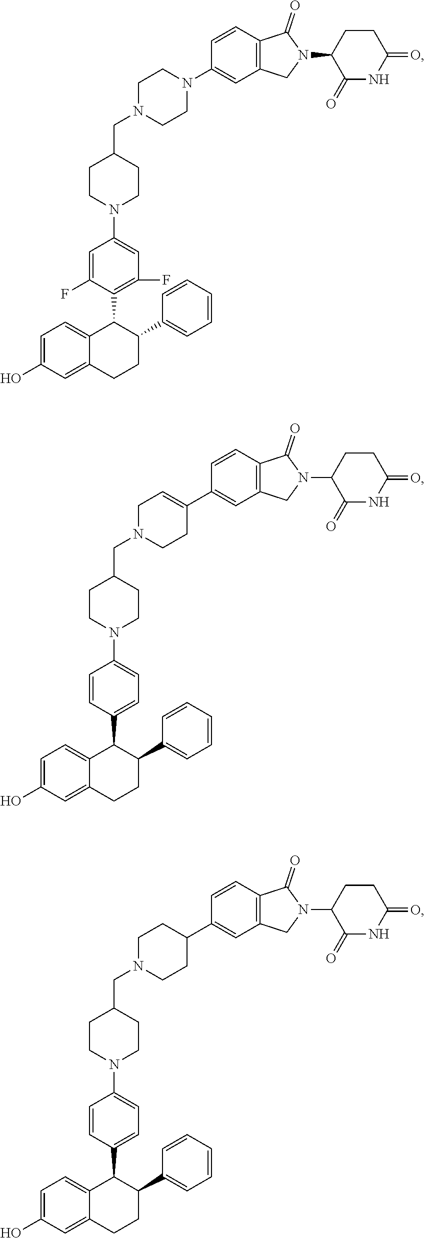 [SMILES: O=C1CC[C@H](N2Cc3cc(N4CCN(CC5CCN(c6cc(F)c([C@H]7c8ccc(O)cc8CC[C@H]7c7ccccc7)c(F)c6)CC5)CC4)ccc3C2=O)C(=O)N1 ; O=C1CCC(N2Cc3cc(C4=CCN(CC5CCN(c6ccc([C@@H]7c8ccc(O)cc8CC[C@@H]7c7ccccc7)cc6)CC5)CC4)ccc3C2=O)C(=O)N1 ; O=C1CCC(N2Cc3cc(C4CCN(CC5CCN(c6ccc([C@@H]7c8ccc(O)cc8CC[C@@H]7c7ccccc7)cc6)CC5)CC4)ccc3C2=O)C(=O)N1] 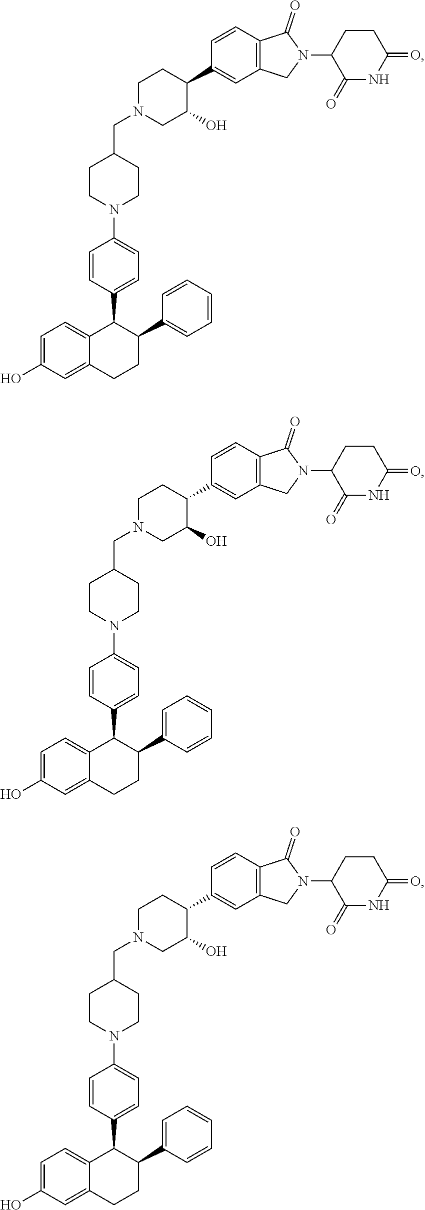 [SMILES: O=C1CCC(N2Cc3cc([C@@H]4CCN(CC5CCN(c6ccc([C@@H]7c8ccc(O)cc8CC[C@@H]7c7ccccc7)cc6)CC5)C[C@H]4O)ccc3C2=O)C(=O)N1 ; O=C1CCC(N2Cc3cc([C@H]4CCN(CC5CCN(c6ccc([C@@H]7c8ccc(O)cc8CC[C@@H]7c7ccccc7)cc6)CC5)C[C@@H]4O)ccc3C2=O)C(=O)N1 ; O=C1CCC(N2Cc3cc([C@H]4CCN(CC5CCN(c6ccc([C@@H]7c8ccc(O)cc8CC[C@@H]7c7ccccc7)cc6)CC5)C[C@H]4O)ccc3C2=O)C(=O)N1] 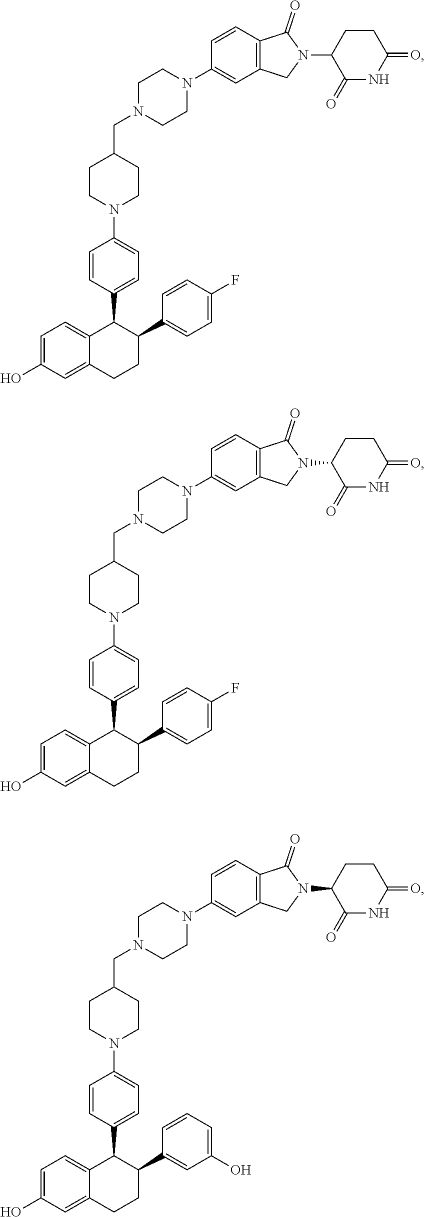 [SMILES: O=C1CCC(N2Cc3cc(N4CCN(CC5CCN(c6ccc([C@@H]7c8ccc(O)cc8CC[C@@H]7c7ccc(F)cc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8CC[C@@H]7c7ccc(F)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7cccc(O)c7)cc6)CC5)CC4)ccc3C2=O)C(=O)N1] 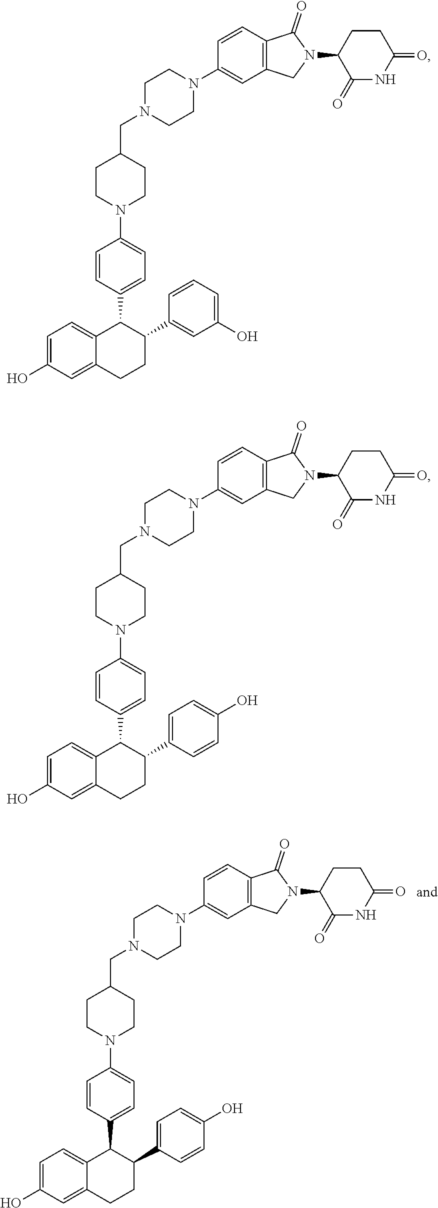 [SMILES: O=C1CC[C@H](N2Cc3cc(N4CCN(CC5CCN(c6ccc([C@H]7c8ccc(O)cc8CC[C@H]7c7cccc(O)c7)cc6)CC5)CC4)ccc3C2=O)C(=O)N1 ; O=C1CC[C@H](N2Cc3cc(N4CCN(CC5CCN(c6ccc([C@H]7c8ccc(O)cc8CC[C@H]7c7ccc(O)cc7)cc6)CC5)CC4)ccc3C2=O)C(=O)N1 ; O=C1CC[C@H](N2Cc3cc(N4CCN(CC5CCN(c6ccc([C@@H]7c8ccc(O)cc8CC[C@@H]7c7ccc(O)cc7)cc6)CC5)CC4)ccc3C2=O)C(=O)N1 ; "and"] 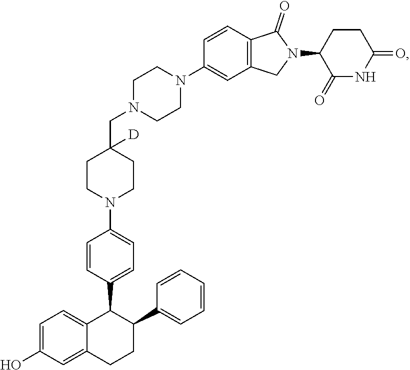 [SMILES: [2H]C1(CN2CCN(c3ccc4c(c3)CN([C@H]3CCC(=O)NC3=O)C4=O)CC2)CCN(c2ccc([C@@H]3c4ccc(O)cc4CC[C@@H]3c3ccccc3)cc2)CC1]
or a pharmaceutically acceptable salt thereof.

122. A pharmaceutical composition comprising a compound of any one of claims 1-121, or a pharmaceutically acceptable salt thereof, and a pharmaceutically acceptable carrier, adjuvant, or diluent.

123. A method of inhibiting ERα signaling in a sample, e.g., in vivo or in vitro, by contacting ERα with a compound of any one of claims 1-121, or a pharmaceutically acceptable salt thereof, or a pharmaceutical composition of claim 122.

124. The method of claim 123, wherein the inhibiting of ERα signaling comprises reducing the signaling activity of ERα by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

125. The method of claim 123, wherein the inhibiting of ERα signaling comprises reducing the signaling activity of ER by at least 1-fold, 1.5-fold, 2-fold, 3-fold, 5-fold, 10-fold, 20-fold, 30-fold, 50-fold, 100-fold, or more, e.g., relative to a reference standard.

126. A method of treating an ERα-mediated disorder in a patient in need thereof, comprising administering to the patient a compound of any one of claims 1-121, or a pharmaceutically acceptable salt thereof, or a pharmaceutical composition of claim 122.

127. The method of claim 126, wherein the ERα-mediated disorder is associated with estrogen receptor accumulation and aggregation.

128. The method of any of claim 126 or 127, wherein the ERα-mediated disorder is cancer or a neoplasia associated with estrogen receptor accumulation and aggregation.

129. The method of claim 128, wherein the ERα-mediated disorder is a disorder mediated by a ERα containing a D544G, Y543S, or L542R mutation.

130. The method of any one of claims 123-129, wherein the ERα-mediated disorder is cancer.

131. The method of any one of claims 123-129, wherein the method comprises the steps of:
(i) identifying a subject in need of such treatment;
(ii) providing a disclosed compound, or a pharmaceutically acceptable salt thereof; and
(iii) administering said provided compound in a therapeutically effective amount to treat, suppress and/or prevent the disease state or condition in a subject in need of such treatment.

132. The method of any one of claims 123-129, wherein the ERα-mediated disorder is breast cancer or uterine cancer.

133. The method of claim 132, wherein the breast cancer is selected from the group consisting of ER+ breast cancer, ER+/HER2-breast cancer, ER+ advanced/metastatic breast cancer, and ER+/HER2-advanced/metastatic breast cancer.

134. The method of any one of claims 123-129, wherein the ERα-mediated disorder is endometriosis.

135. A compound or pharmaceutically acceptable salt, solvate, stereoisomer, or tautomer thereof, according to any one of claims 1-121, or a pharmaceutically acceptable salt thereof, or a pharmaceutical composition of claim 122, for use as a medicament.

136. A compound or pharmaceutically acceptable salt, solvate, stereoisomer, or tautomer thereof, according to any one of claims 1-121, or a pharmaceutically acceptable salt thereof, or a pharmaceutical composition of claim 122, for use in the treatment of an ERα-mediated disorder.
